(19) United States
(12) Patent Application Publication
(10) Pub. No.: US 2026/0199510 A1
(43) Pub. Date: Jul. 16, 2026

(54) AN ANTIBODY-DRUG CONJUGATE HAVING TWO OR MORE FUNCTIONAL SMALL MOLECULE COMPOUNDS FOR ENHANCED TREATMENT OF REFRACTORY DISEASES

(71) Applicants: Robert Yongxin ZHAO, Lexington, MA (US); HANGZHOU SEEHE BIOTECHNOLOGY CO., LTD, Hangzhou, Zhejiang (CN); HANGZHOU DAC BIOTECH CO., LTD, Hangzhou, Zhejiang (CN)

(72) Inventors: Robert Yongxin ZHAO, Lexington, MA (US); Qingliang YANG, Hangzhou, Zhejiang (CN); Yuanyuan HUANG, Hangzhou, Zhejiang (CN); Hangbo YE, Hangzhou, Zhejiang (CN); Lingli ZHANG, Hangzhou, Zhejiang (CN); Huihui GUO, Hangzhou, Zhejiang (CN); Junxiang JIA, Hangzhou, Zhejiang (CN); Lu BAI, Hangzhou, Zhejiang (CN); Juan WANG, Hangzhou, Zhejiang (CN); Wenjun LI, Hangzhou, Zhejiang (CN)

(73) Assignees: HANGZHOU SEEHE BIOTECHNOLOGY CO., LTD, Hangzhou, Zhejiang (CN); HANGZHOU DAC BIOTECH CO., LTD., Hangzhou, Zhejiang (CN)

(21) Appl. No.: 19/131,225

(22) PCT Filed: May 22, 2024
(86) PCT No.: PCT/CN2024/094651

§ 371 Date: May 20, 2025

(30) Foreign Application Priority Data
May 24, 2023 (WO) PCT/CN2023/096066

(51) Int. Cl. A61K 47/68; A61K 31/196; A61K 31/282; A61K 31/337; A61K 31/407; A61K 31/4745; A61K 31/475; A61K 31/513; A61K 31/519; A61K 31/7048; A61K 33/243; A61P 35/00

(52) U.S. Cl. CPC A61K 47/6889; A61K 31/196; A61K 31/282; A61K 31/337; A61K 31/407; A61K 31/4745; A61K 31/475; A61K 31/513; A61K 31/519; A61K 31/7048; A61K 33/243; A61K 47/6803; A61K 47/68031; A61K 47/68033; A61K 47/68035; A61K 47/68037; A61K 47/6805; A61K 47/6809; A61P 35/00

(57) ABSTRACT

An antibody-drug conjugate containing a side chain of a functional small molecule for enhancement of targeted treatment of cancers and refractory diseases is provided. The preparation of such conjugate, pharmaceutical compositions, and methods in treatment of refractory diseases are also provided.

### INFORMATION OF PRIORITY

The present application claims the benefit of PCT/CN2023/096066 field on May 24^{th}, 2023, which is incorporated herein reference.

### BACKGROUND

Antibody-drug conjugates (ADCs) that combine the target specificity of a monoclonal antibody (mAb) with the potent cytotoxic drugs are a rapidly expanding class of therapeutic agents against cancers. There are 15 ADCs so far approved as monotherapy or combinational therapy for several solid and liquid cancers. Due to the spectacular success of 9 of the 15 ADC approved in the past 5 years for some highly treatment-refractory diseases, over a 200 new ADCs are now in clinical trials encompassing a wide variety of tumor types according to https://www.clinicaltrials.gov. Despite the explosion of interest in ADCs, challenges remain to expand their therapeutic index (with greater efficacy and less toxicity) and applications in treatment of various cancers (Dean, A. Q., et al, mAbs, 2021, 13 (1), 1951427). Thus, more and more ADCs are combined with chemotherapeutical drugs, immunotherapy compounds and others for the expansion in the treatment (Li, Y, et al, Am J Cancer Res. 2023, 13(1):161-17; Ceci, C., et al, Pharmacol. 2022, 236:108106; Gerber, H. P, et al, Biochem Pharmacol. 2016, 102:1-6.). However, the half-lives of chemotherapeutical drugs are normally quite short and they have to be given more frequently to patients, therefore leading to more toxicity in synergistic use with ADCs (in comparison with application of the monotherapy of ADCs) (Fuentes-Antras, J., et al, Trends Cancer 2023, 9(4): 339-354). We invented an ADC contains a group of an affinity small molecule, such as an affinity peptide or a cell penetrating peptide that have enhanced affinity with the antibody, resulting in broader treatment of the tumors (PCT/CN2023/096066). Here in this application, we disclose an ADC covalently linked one or more functional small molecules which have synergies of affinity with an antibody and/or the broader potency with a cytotoxic drug, resulting in enhancement of the targeted treatment of various cancers and refractory diseases. Further disclosed are preparation of the conjugate, pharmaceutical compositions, screening, and medical treatment methods.

### BRIEF SUMMARY OF THE INVENTION

The present invention provides an antibody-drug conjugate (ADC) with a branch linked chemical drugs or functional small molecules for the enhancement of targeted treatment of cancers and refractory diseases. The preferred formulas of the ADCs are represented as (I), (II), (III) and (IV) below:

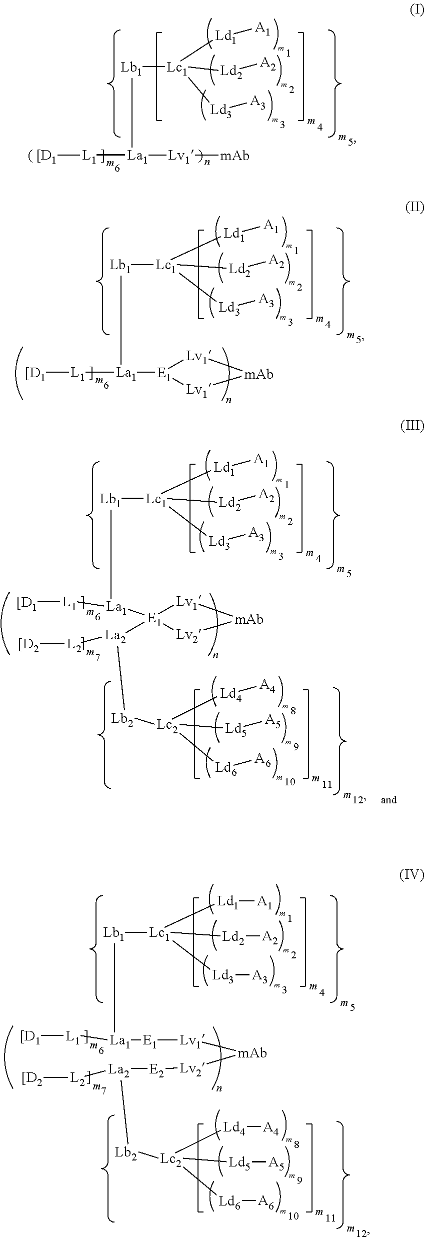

### Wherein,

D₁ and D₂ are a cytotoxic agent; mAb is an antibody, an antibody like protein or a cell-binding protein; n is 1, 2, 3, 4, 5, 6, 7, 8, 9, 10, 11, 12, 13, 14, 15, 16, 17, 18, 19 or 20, and these numbers may have a decimal;
L₁, L₂, La₁, La₂, Lb₁, Lb₂, Lc₁, Lc₂, Ld₁, Ld₂, Ld₃, Ld₄, Ld₅, and Ld₆ are a linker, which are independently selected from O, NH, S, N, NH—NH, N—N, N(R₃), N(R₃)N(R_{3′}), C(═O)N, C(═O)NH, C(═O)N—N, C₁-C₈ of alkyl (alkylene); C₂-C₅ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, aminoalkylcarboxyl or oxylalkylcarboxyl; or 1-8 natural or unnatural amino acids described in the definition; or polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃][(CH₂CH₂O)ₚ>R₃,], or (OCH₂CH₂)ₚCOOR₃, or CH₂CH₂(OCH₂CH₂)ₚCOOR₃, wherein p and p′ are independently an integer selected from 0 to about 100, or combination thereof; wherein R₃ and R_{3′} are independently H, C(═O)H, C(═O)CH₃, C₁-C₈ of alkyl; or combination above thereof;
E₁ and E₂ are a joint group that link two reactivable groups, Lv₁ and Lv₂, which preferably can react to a thiol, amino, phenol, ketone, aldehyde, alkyne, hydroxyl, or carboxylic acid group in an antibody to form a group of Lv₁′ and Lv₂′ respectively. E₁ and E₂ are independently selected from CH, CH₂, CH—CH, NH, NHNH, N(R₃), N(R₃)N(R₃,), N═N, N—N, P, P(═O), S, Si, C₂-C₈ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing1~4 units of aminoacids, preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine; more detail structures are described in the specification of the invention. In addition, E₁ can be absent, thus La₁ or/and La₂ can directly link to a group of Lv₁′ or Lv₂′;
m₁, m₂, m₃, m₄, m₅, m₆, m₇, m₈, m₉, m₁₀, m₁₁ and m₁₂ are independently 1, 2, 3, 4, 5, 6, 7, 8, 9, or 10 and these numbers may have a decimal; in addition, m₂, m₃, m₈, m₉ and/or m₁₀ can be 0, thus Ld₂ A₂, Ld₃—A₃, Ld₅—A₅, and/or Ld₆—A₆ can be absent;
A₁, A₂, A₃, A₄, A₅ and A₆ are a functional small molecule, independently selected from an affinity ligand such as for bombesin receptors/neurotensin receptors (including neuropeptide-Y receptors), a cell-penetrating peptide; and/or a nucleoside antimetabolite/analog; which have either the synergy with D₁ and/or D₂, or enhanced affinity for mAb. The detail structures are described in the specification of the invention.
wherein Lv₁′ and Lv₂′ are a group that are resulted from reaction of Lv₁ and Lv₂ to an amino acid of an antibody or a cell-binding protein independently. The detail structures of Lv₁′ and Lv₂′ are described in the specification of the invention.

The present invention also provides an antibody-drug conjugate (ADC), comprising a monoclonal antibody, or an antigen-binding fragment thereof, a cytotoxin, and a linker having a functional small molecule, and/or an affinity ligand, such as for bombesin receptors/neurotensin receptors (including neuropeptide-Y receptors), and/or a cell-penetrating peptide, and or an affinity peptide, such as the programmed death ligand-1 (PD-L1, or CD274), expressed on tumor cells and tumor-infiltrating immune cells, resulting in enhancement of targeted treatment of cancers and refractory diseases. In a further embodiment the antigen binding proteins are conjugated to a potent toxin such as a tubulysin analog, a camptothecin (CPT) analog, a PBD dimer, an eribulin, an auristatin analog, a duocarmycin analog, or an anthracycline analog.

In addition, the invention provides compositions comprising the foregoing antibody-drug conjugate, and a pharmaceutically acceptable carrier, and methods of the targeted treatment of various cancers and refractory diseases.

### BRIEF DESCRIPTION OF THE SEVERAL VIEWS OF THE DRAWING(S)

FIG. 1 shows the synthesis of a linker containing a DUPA component.

FIG. 2 shows the synthesis of a conjugate of tubulysin analog containing a DUPA component.

FIG. 3 shows the synthesis of a conjugate of tubulysin analog containing a DUPA component.

FIG. 4 shows the synthesis of a conjugate of tubulysin analog containing a DUPA component.

FIG. 5 shows the synthesis of a conjugate of tubulysin analog containing a DUPA component.

FIG. 6 shows the synthesis of a conjugate of tubulysin analog containing a DUPA component.

FIG. 7 shows the synthesis of a conjugate of tubulysin analog containing dual DUPA components.

FIG. 8 shows the synthesis of a conjugate of tubulysin analog containing dual DUPA components.

FIG. 9 shows the synthesis of a conjugate of tubulysin analog containing dual DUPA components.

FIG. 10 shows the synthesis of a conjugate of tubulysin analog containing dual DUPA components.

FIG. 11 shows the synthesis of a conjugate of tubulysin analog containing dual DUPA components.

FIG. 12 shows the synthesis of a conjugate of tubulysin analog containing dual DUPA components.

FIG. 13 shows the synthesis of a linker component containing tri-gemcitabine analogs.

FIG. 14 shows the synthesis of a CPT analog with a conjugatable linker having tri-gemcitabines.

FIG. 15 shows the synthesis of a CPT-ADC with a linker containing tri-gemcitabines.

FIG. 16 shows the synthesis of a CPT-ADC with a linker containing tri-gemcitabines.

FIG. 17 shows the synthesis of a CPT-ADC with a linker containing tri-gemcitabines.

FIG. 18 shows the synthesis of a CPT-ADC with a linker containing tri-gemcitabines.

FIG. 19 shows the synthesis of a CPT-ADC with a linker containing tri-gemcitabines.

FIG. 20 shows the synthesis of a CPT-ADC with a linker containing tri-gemcitabines.

FIG. 21 shows the synthesis of a CPT analog with a conjugatable linker having tri-gemcitabines.

FIG. 22 shows the synthesis of a CPT-ADC with a linker containing tri-gemcitabines.

FIG. 23 shows the synthesis of a CPT-ADC with a linker containing tri-gemcitabines.

FIG. 24 shows the synthesis of a CPT analog with a conjugatable linker having tri-gemcitabines.

FIG. 25 shows the synthesis of a CPT-ADC with a linker containing tri-gemcitabines.

FIG. 26 shows the synthesis of a CPT-ADC with a linker containing tri-gemcitabines.

FIG. 27 shows the synthesis of a CPT-ADC with a linker containing tri-gemcitabines.

FIG. 28 shows the synthesis of a CPT-ADC with a linker containing tri-gemcitabines and a CPT payload/linker complex.

FIG. 29 shows the synthesis of an ADC with a bis-linker containing dual-payloads of tubulysin analog and CPT analog.

FIG. 30 shows the synthesis of an ADC with a bis-linker containing dual-payloads of tubulysin analog and CPT analog.

FIG. 31 shows the synthesis of an ADC with a bis-linker containing dual-payloads of tubulysin analog and CPT analog, and dual CPT payload/linker complexes.

FIG. 32 shows the synthesis of an ADC with a bis-linker containing dual-payloads of CPT analogs.

FIG. 33 shows the synthesis of an ADC with a bis-linker containing dual-payloads of tubulysin analog and CPT analog.

FIG. 34 shows the synthesis of an ADC with a bis-linker containing dual-payloads of tubulysin analog and CPT analog.

FIG. 35 shows the synthesis of an ADC with a linker containing dual-payloads of tubulysin analog and CPT analog.

FIG. 36 shows the synthesis of an ADC with a linker containing dual-payloads of tubulysin analog and CPT analog.

FIG. 37 shows the synthesis of an ADC with a linker containing dual-payloads of tubulysin analog and CPT analog.

FIG. 38 shows the synthesis of an ADC with a linker containing dual-payloads of tubulysin analog and CPT analog and a CPT-payload/linker complex containing a cell penetrating cyclopeptide.

FIG. 39 shows the synthesis of a CPT-ADC with a bis-linker containing a cell penetrating cyclopeptide and a CPT/linker complex containing a cell penetrating cyclopeptide.

FIG. 40 shows the synthesis of a CPT-ADC with a bis-linker containing a cell penetrating cyclopeptide and a tubulysin conjugate with a linker containing a cell penetrating cyclopeptide.

FIG. 41 shows the synthesis of a tubulysin-linker complex containing a cell penetrating cyclopeptide.

FIG. 42 shows the synthesis of a tubulysin conjugate with a linker containing a cell penetrating cyclopeptide and CPT payloads-linker complex containing a cell penetrating cyclopeptide.

FIG. 43 shows the synthesis of CPT- conjugate with a linker containing a cell penetrating cyclopeptide and CPT payloads-linker complex containing a cell penetrating cyclopeptide.

FIG. 44 shows the synthesis of a CPT-ADC with a linker containing a cell penetrating cyclopeptide and CPT payloads-linker complex containing a cell penetrating cyclopeptide.

FIG. 45 shows the synthesis of a CPT payload-linker complex containing a DUPA.

FIG. 46 shows the synthesis of CPT- conjugate with a linker containing a DUPA.

FIG. 47 shows the synthesis of CPT- a dual maleimido linker complex containing DUPA.

FIG. 48 shows the synthesis of CPT- conjugate with a linker containing a DUPA.

FIG. 49 shows the synthesis of CPT- conjugate with a linker containing a DUPA.

FIG. 50 shows the synthesis of CPT- conjugate with a linker containing bis DUPAs.

FIG. 51 shows the synthesis of CPT- conjugate with a linker containing a DUPA.

FIG. 52 shows the synthesis of CPT- conjugate with a linker containing a DUPA.

FIG. 53 shows the synthesis of CPT- conjugate with a linker containing a DUPA.

FIG. 54 shows the synthesis of CPT- conjugate with a linker containing a DUPA and a linker component containing tri-gemcitabines.

FIG. 55 shows the synthesis of CPT- conjugate with a linker containing tri-gemcitabines.

FIG. 56 shows the synthesis of CPT- conjugate with a linker containing a gemcitabines.

FIG. 57 shows the synthesis of CPT- conjugate with a linker containing tri-gemcitabines.

FIG. 58 shows the synthesis of CPT- conjugate with a linker containing bis-gemcitabines.

FIG. 59 shows the synthesis of CPT- conjugate with a linker containing bis-gemcitabines.

FIG. 60 shows the synthesis of CPT- conjugate with a linker containing a cell penetrating cyclopeptide.

FIG. 61 shows the synthesis of CPT- conjugate with a linker containing a cell penetrating cyclopeptide.

FIG. 62 Illustrates change in tumor volume in a C₄-2B prostate cancer cell xenograft mouse model in response to a single dose (4.0 mg/Kg) treatment with Steap1 ADCs (GGFG-Dxd, C025, C030, C043, C072, C082, C095, C284, C324, C358, C361, C366, and C383, DARs indicated in the Figure), in comparison to PBS buffer (the control). The figure indicates that all the 13 conjugates had antitumor activity, and the orders of the antitumor activity are: Dxd <C284<C358<C366<C324<C082<C361<C383<C030<C025<C043<C072<C095. It also demonstrated that the conjugates with affinity ligands in the payload/linker complexes of the invention can improve the antitumor activity in vivo, resulting in better in vivo activity than the well-known GGFG-Dxd complex.

FIG. 63 Illustrates change in tumor volume in a A431 cancer cell xenograft mouse model in response to a single dose (6.0 mg/Kg) treatment with Mucl/EGFR bispecific ADCs (GGFG-Dxd, C342, C467, C571a, C571c, C574c, C574d, C572d, C572i, C574q, C572q, C662, and C675. DARs indicated in the Figure), in comparison to PBS buffer (the control). The figure indicates that all the 13 conjugates had antitumor activity, and the orders of the antitumor activity are: GGFG-Dxd <C342<C467<C571a <C571c <C574c <C574d <C572d <C572i <C574q <C572q <C662<C675. It also demonstrated that the extra small molecules in the payload/linker complexes of the invention can improve the antitumor activity in vivo, and with the same category of the payloads, the conjugates containing the extra small molecules in the payload/linker complexes of the invention had better antitumor activity than the well-known GGFG-Dxd conjugate.

FIG. 64 Illustrates change in tumor volume in a HCC827 cancer cell xenograft mouse model in response to a single dose (3.0 mg/Kg) treatment with Trop2 ADCs (GGFG-Dxd, C150, C141, C423, C190, C222, C433, C211, C416, C534, C578f, C578k, C578n, and C580m, DARs indicated in the Figure), in comparison to PBS buffer (the control). The figure indicates that all the 14 conjugates had antitumor activity, and the orders of the antitumor activity are: GGFG-Dxd <C150<C141<C423<C190<C222<C433<C211<C416<C534<C578f <C578k <C578n <C580m. It also demonstrated that the extra small molecules in the payload/linker complexes of the invention can improve the antitumor activity in vivo, and with the same category of the payloads, the conjugates containing the extra small molecules in the payload/linker complexes of the invention had better antitumor activity than the well-known GGFG-Dxd conjugate.

FIG. 65 Illustrates change in tumor volume in a SU86.86 cancer cell xenograft mouse model in response to a single dose (2.5 mg/Kg) treatment with bispecific Her2 ADCs (GGFG-Dxd, C254, C314, C572o, C572k, C439, C534, C576c, C577c, C580c, C580e, C580g, and C658. DARs indicated in the Figure), in comparison to PBS buffer (the control). The figure indicates that all the 13 conjugates had antitumor activity, and the orders of the antitumor activity are: GGFG-Dxd <C254<C314<C572o <C572k <C439<C534<C576c <C577c <C580c <C580e <C580g <C658. It also demonstrated that the extra small molecules in the payload/linker complexes of the invention can improve the antitumor activity in vivo, and with the same category of the payloads, the conjugates containing the extra small molecules in the payload/linker complexes of the invention had better antitumor activity than the well-known GGFG-Dxd conjugate.

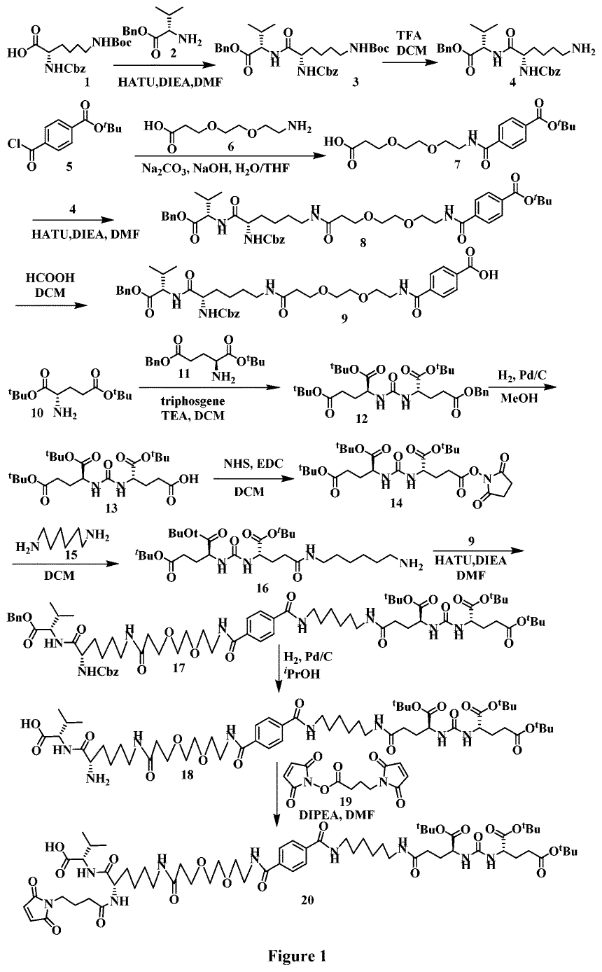

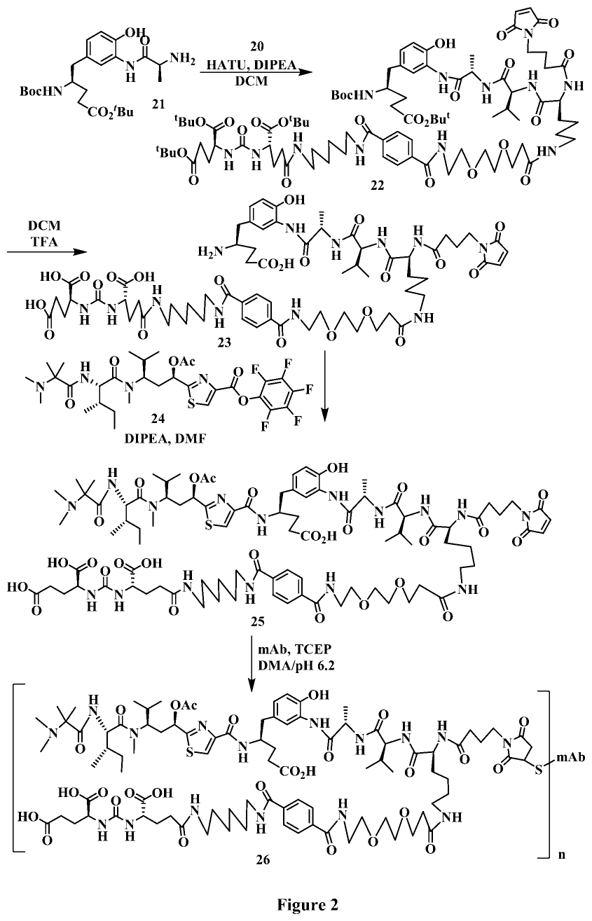

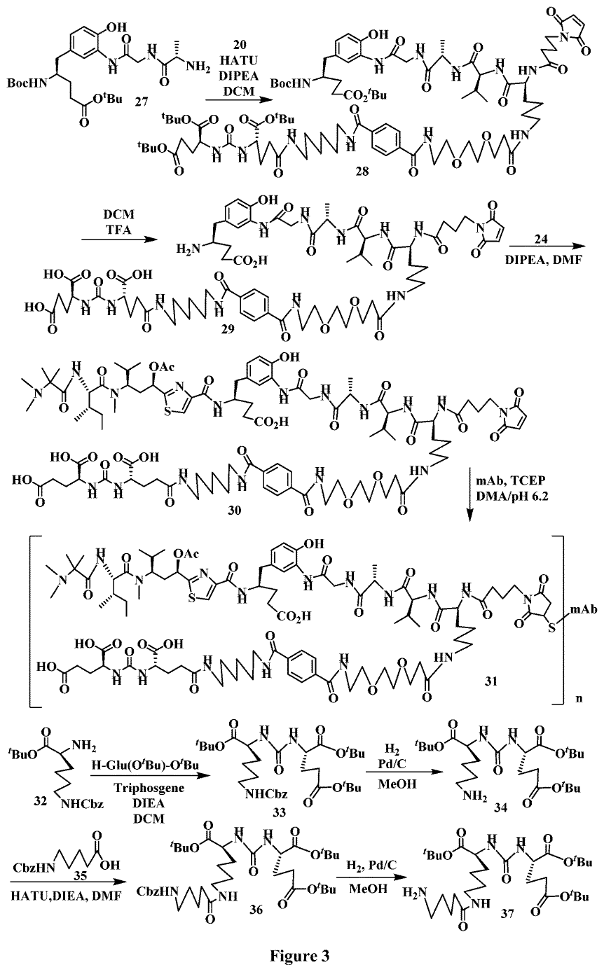

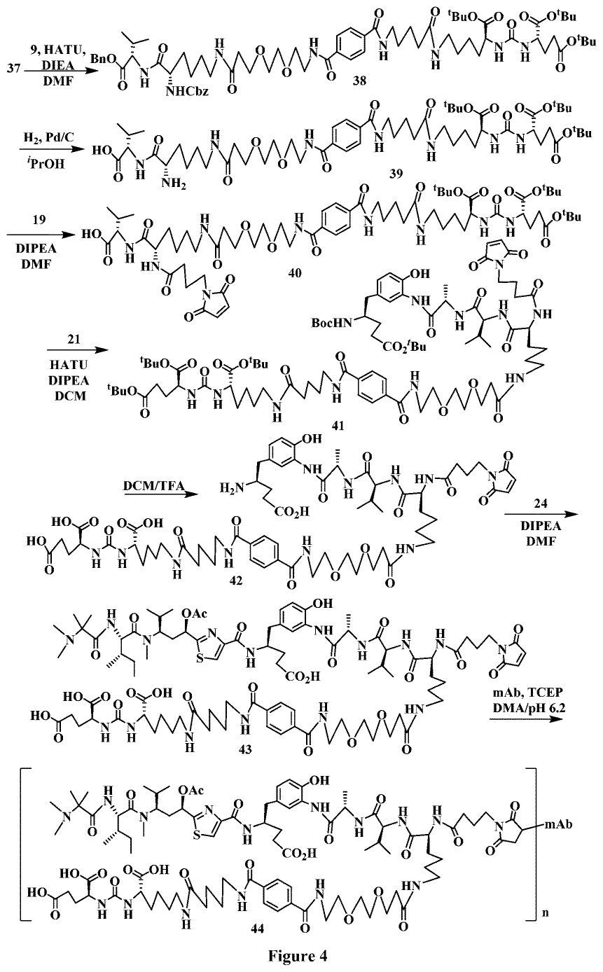

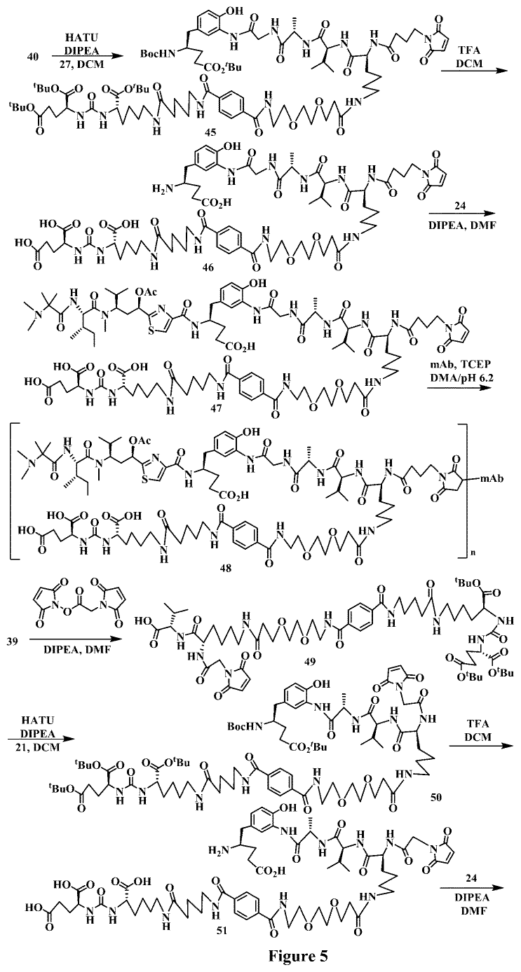

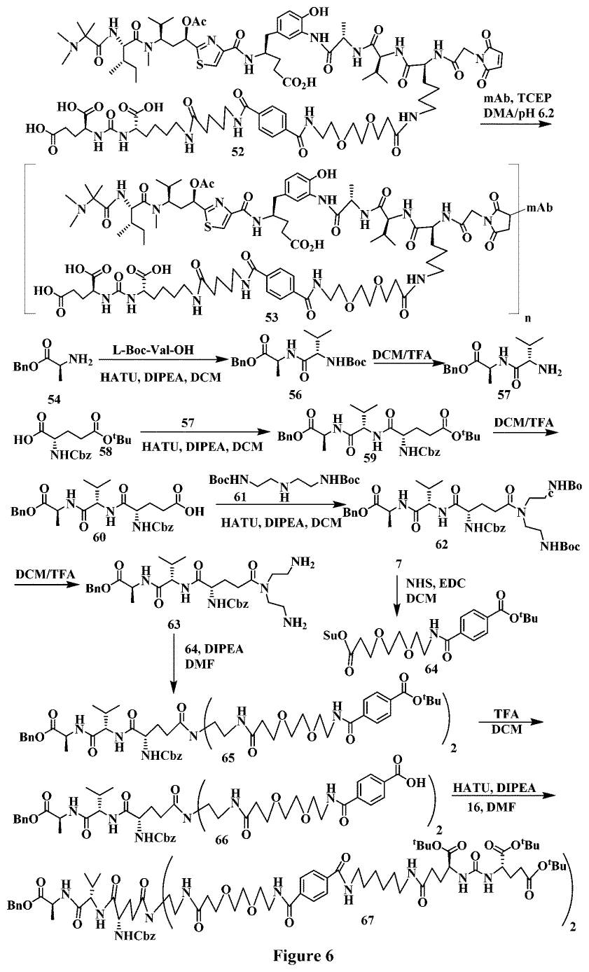

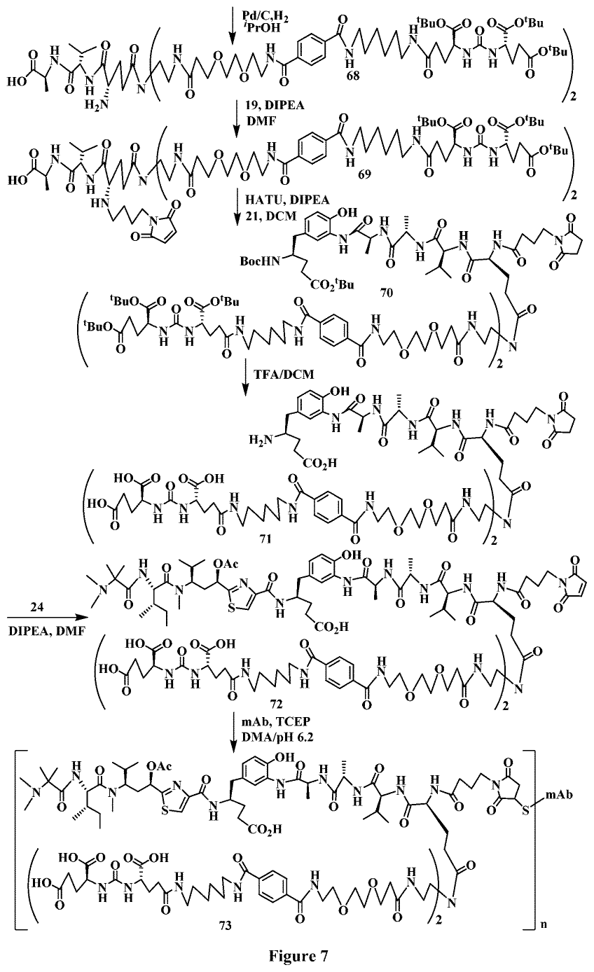

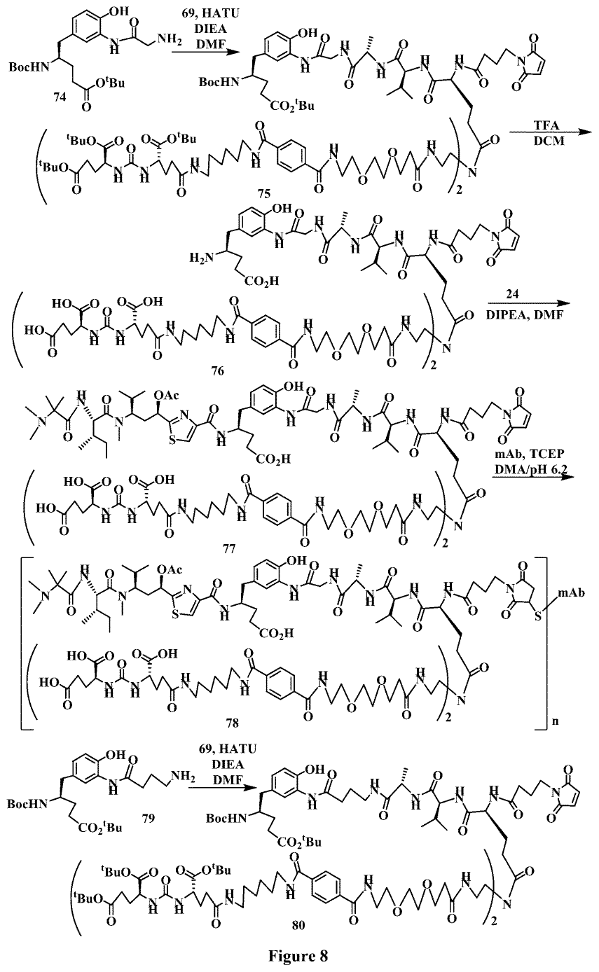

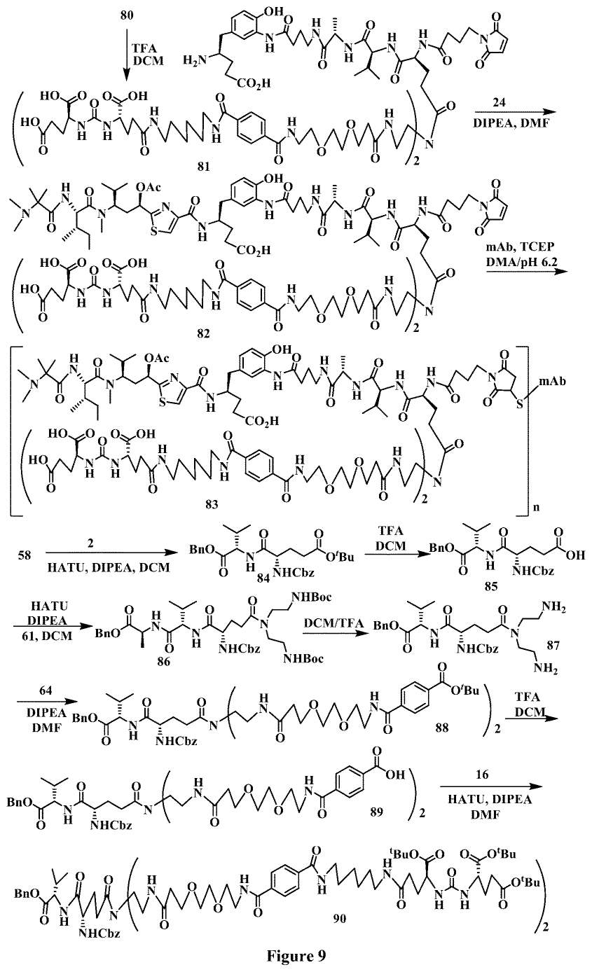

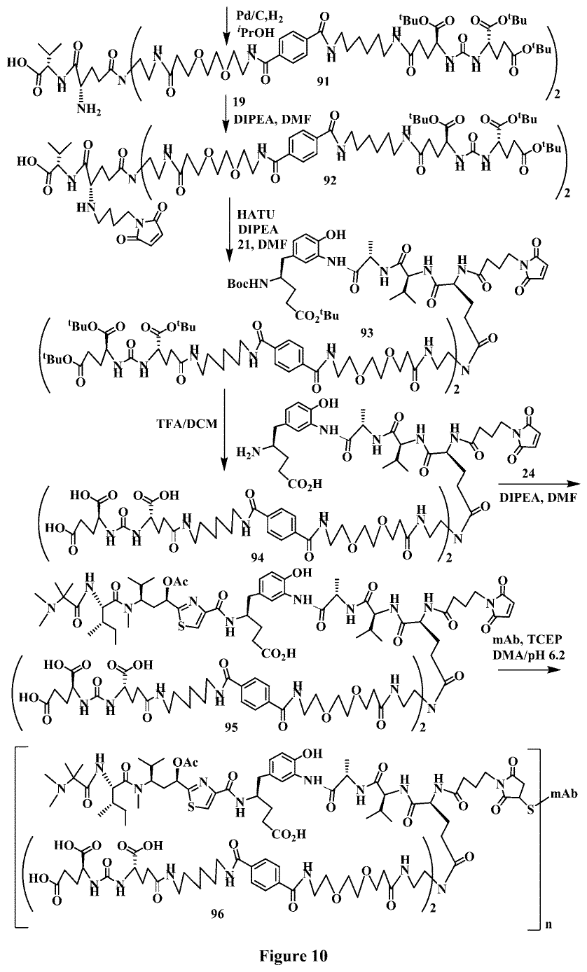

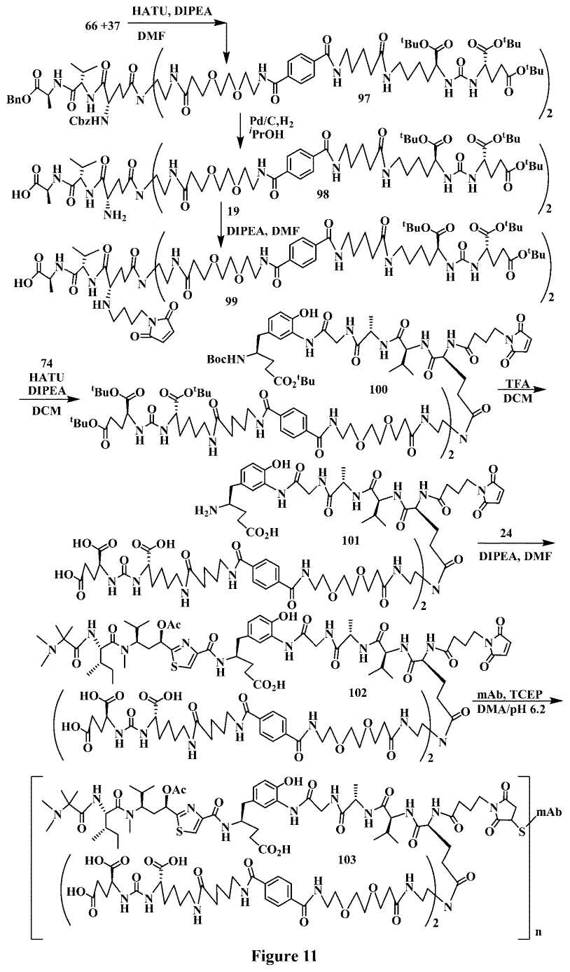

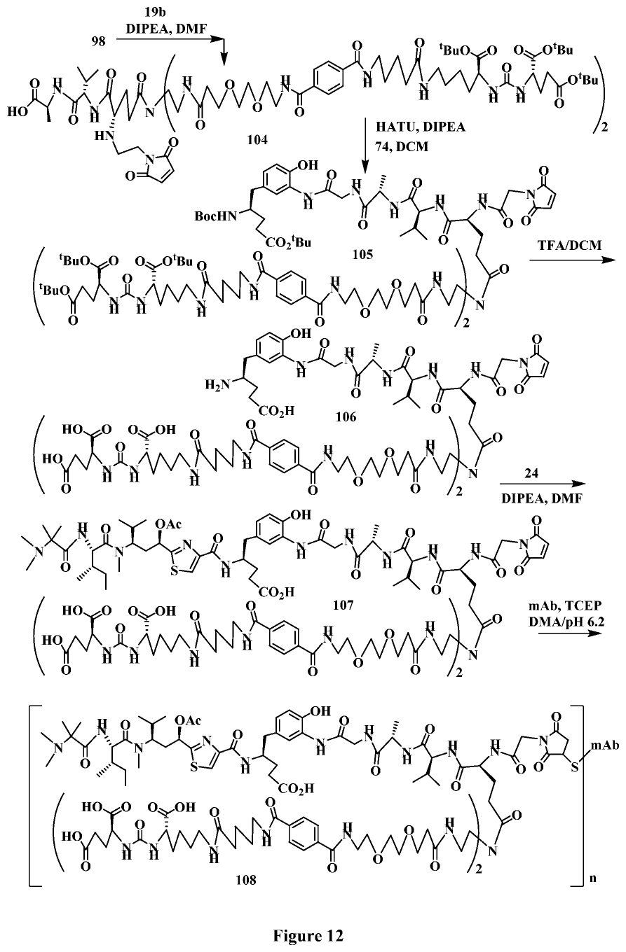

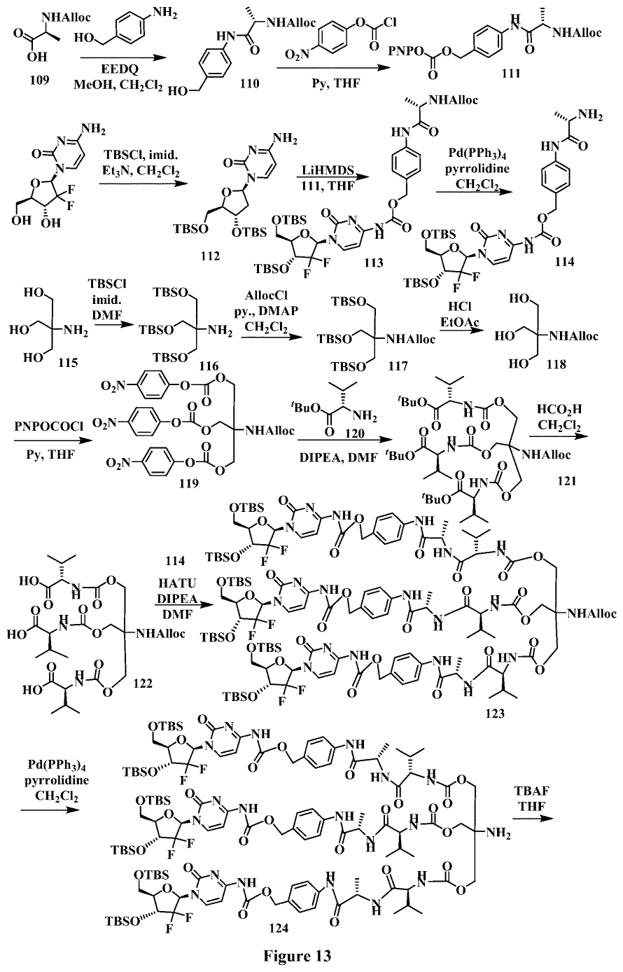

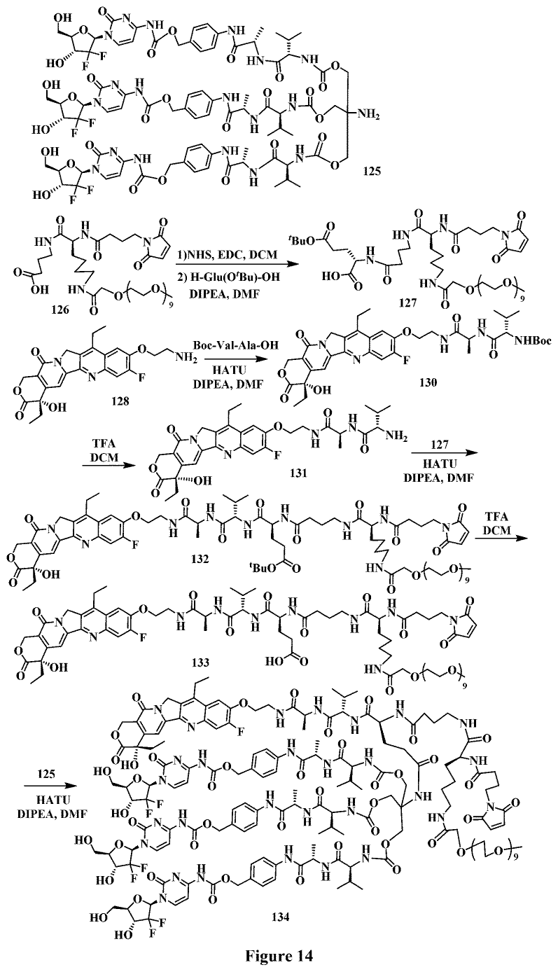

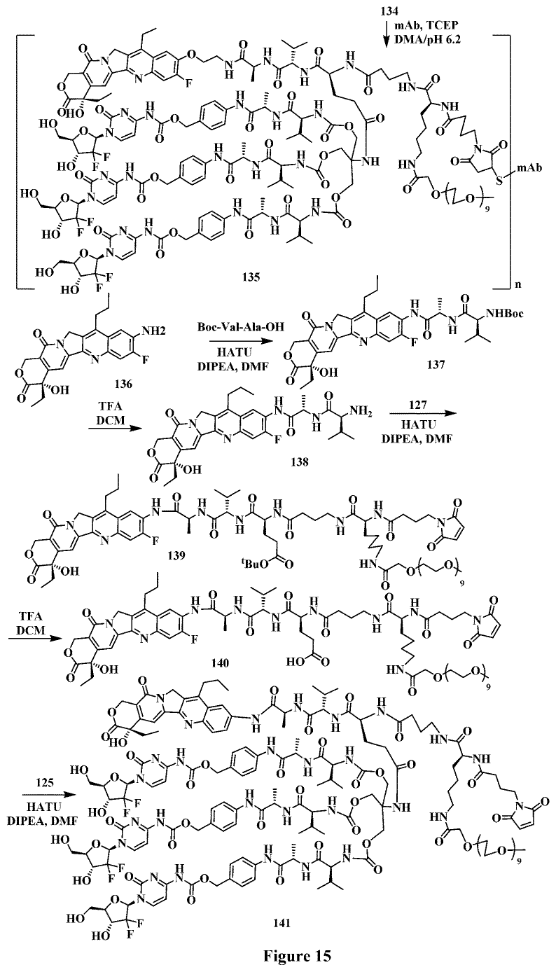

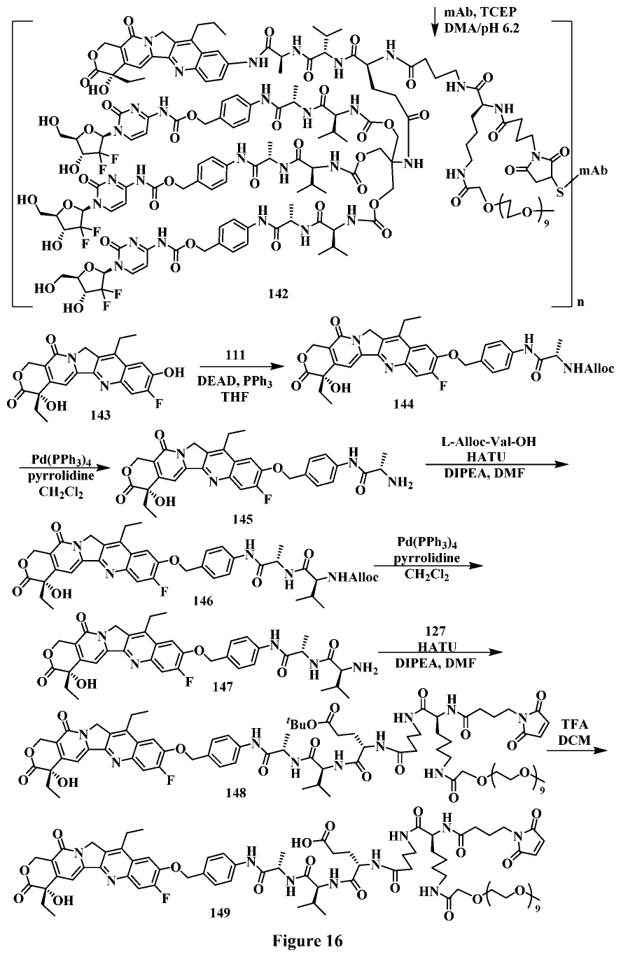

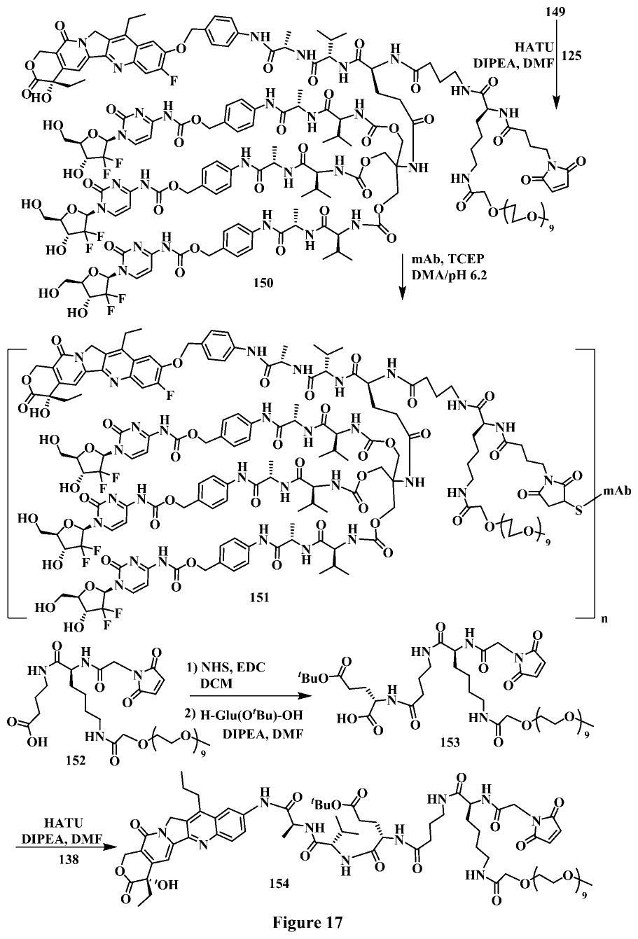

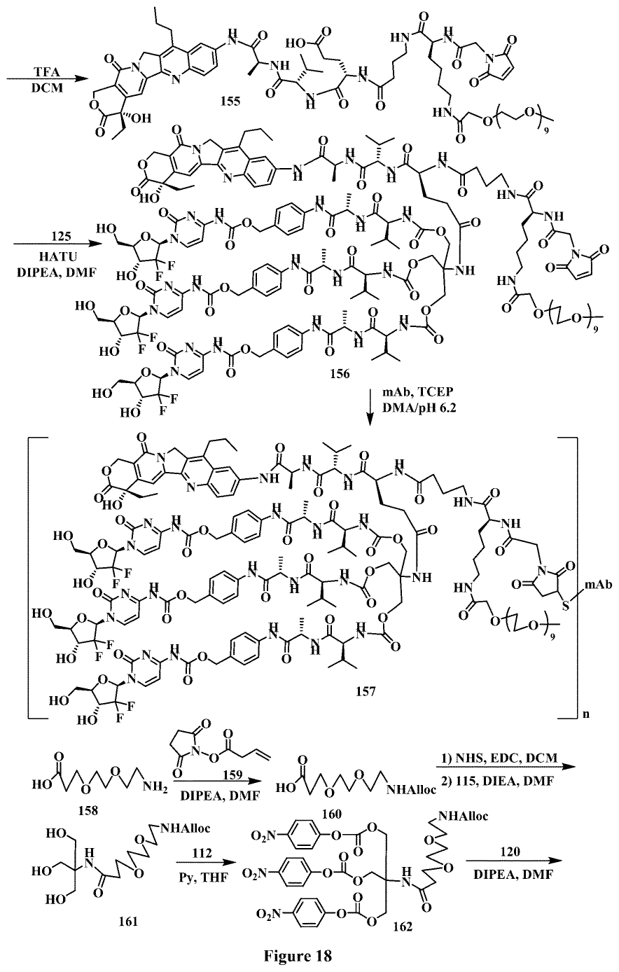

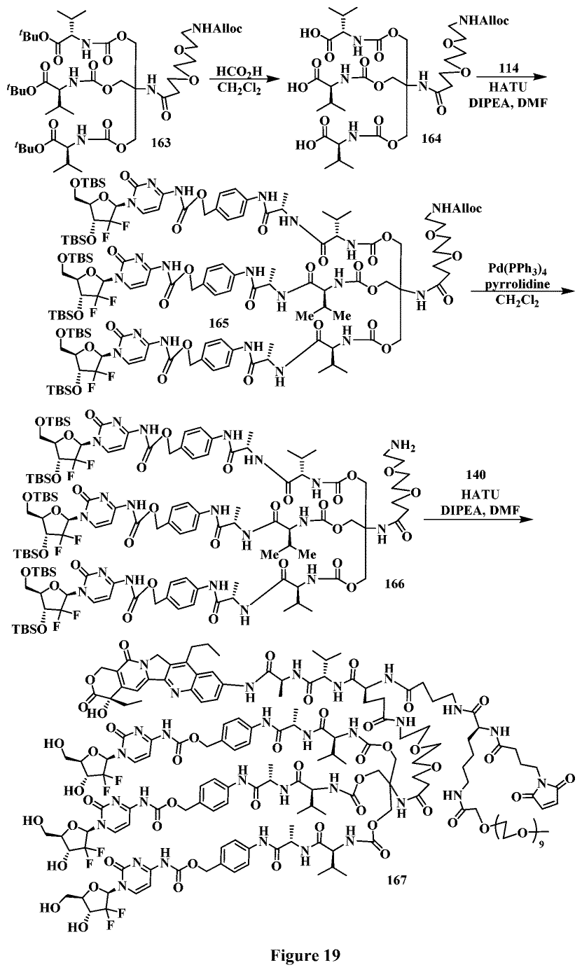

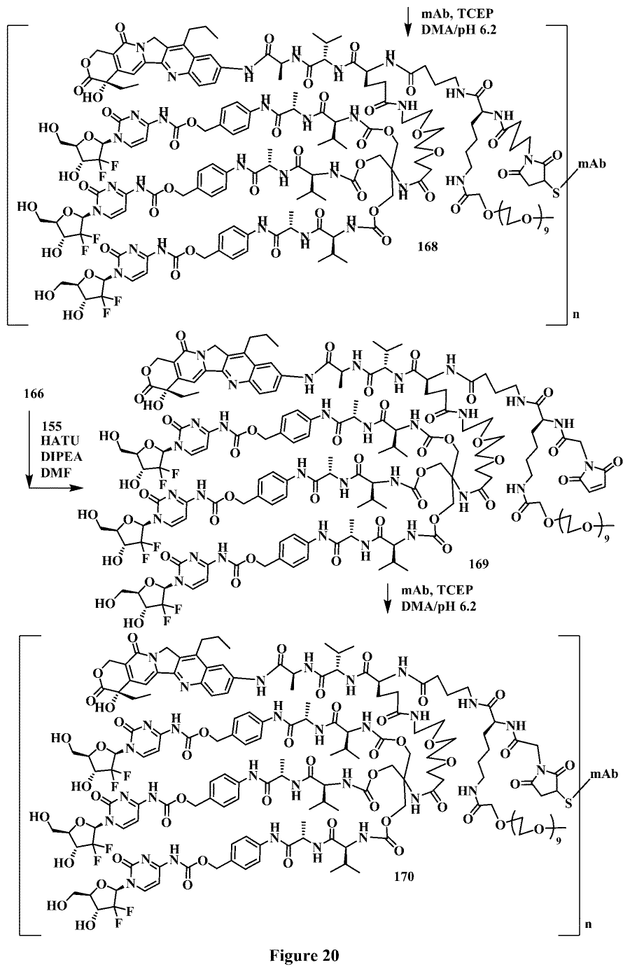

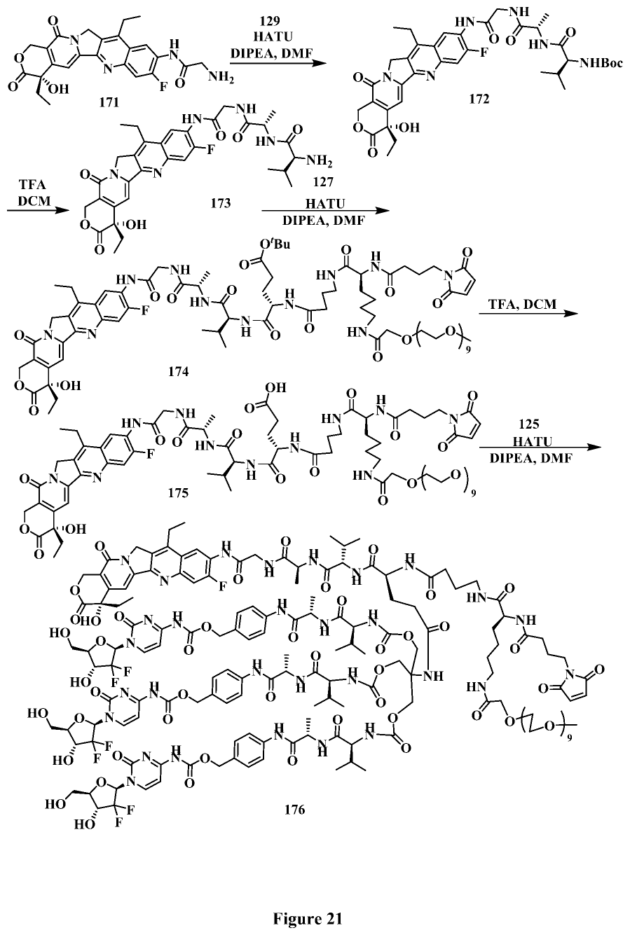

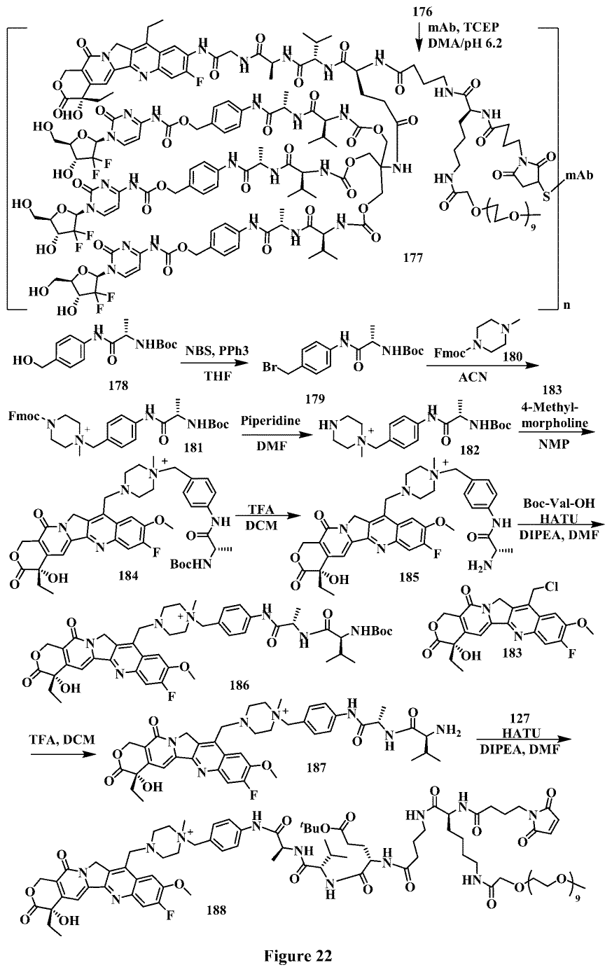

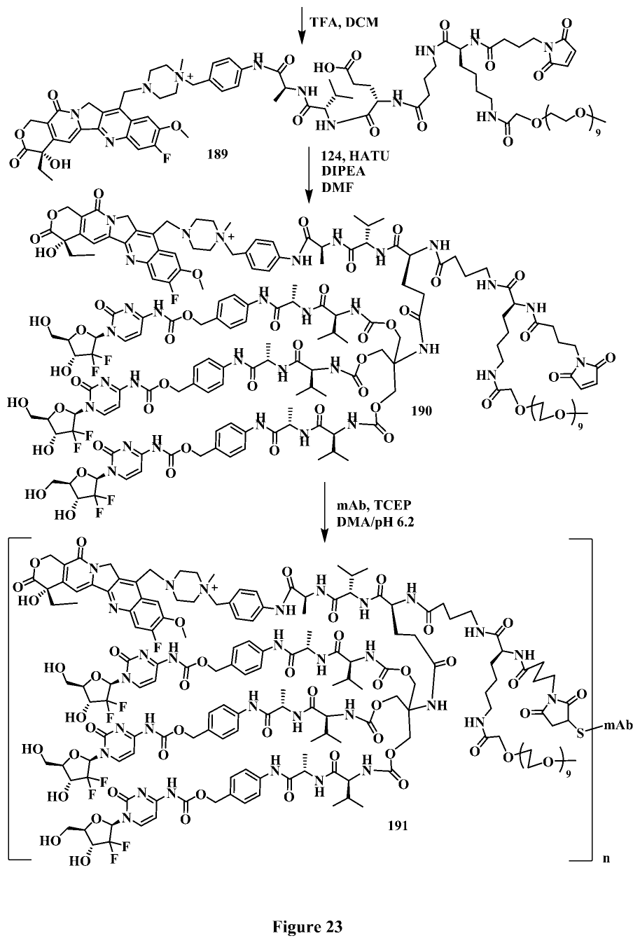

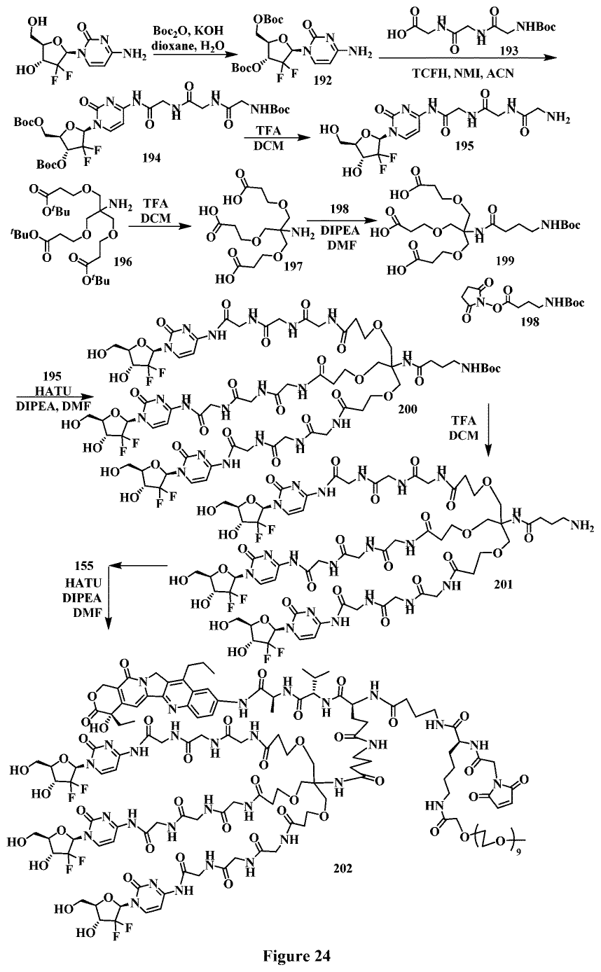

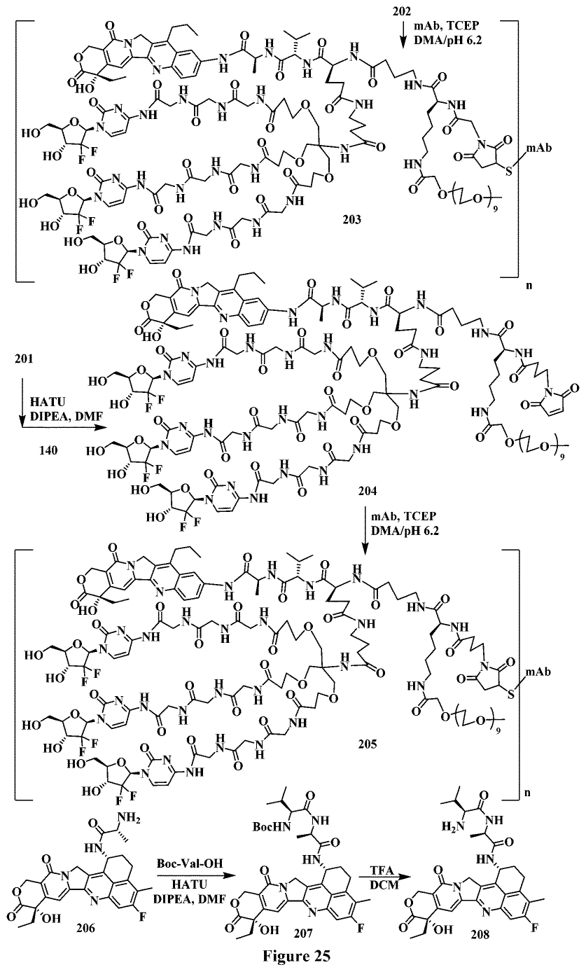

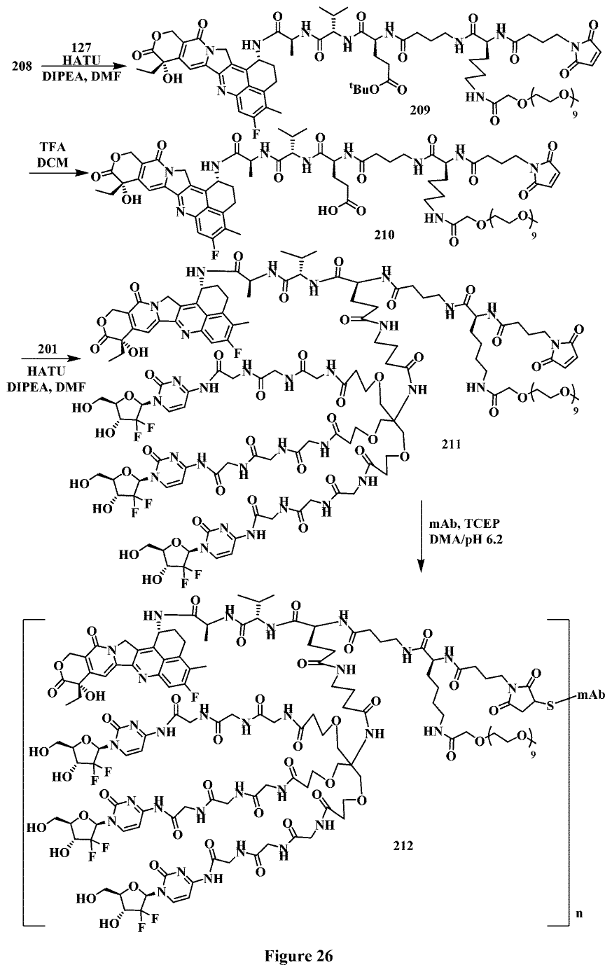

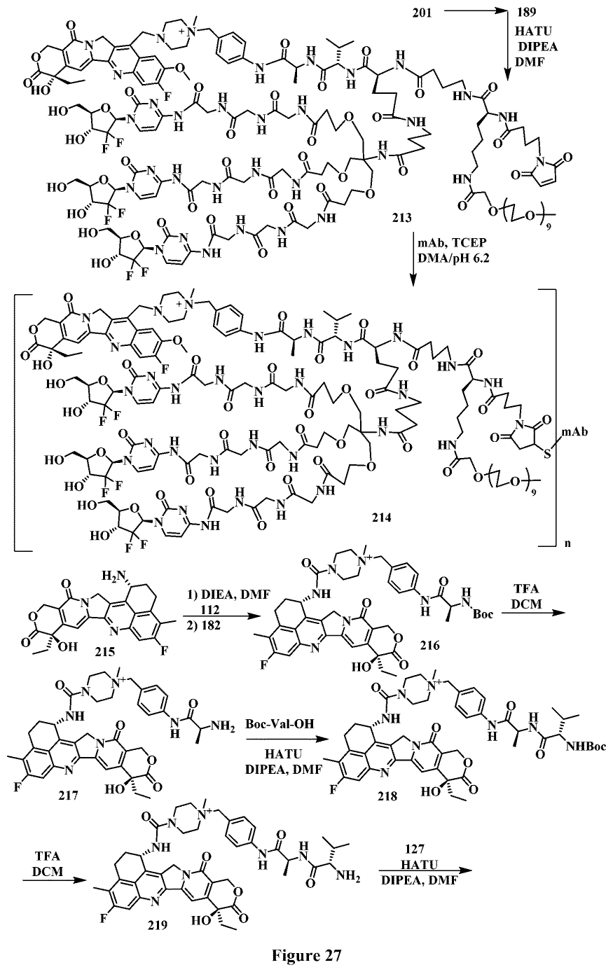

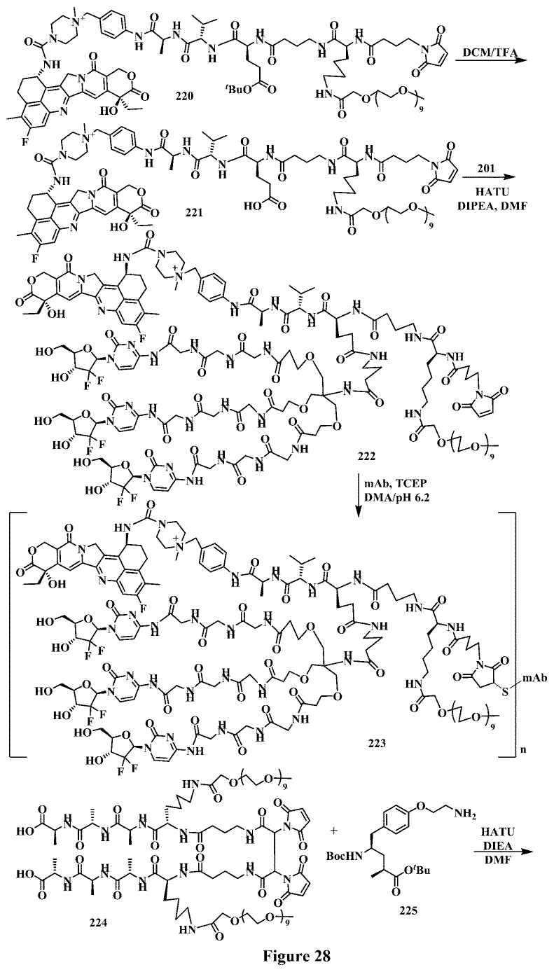

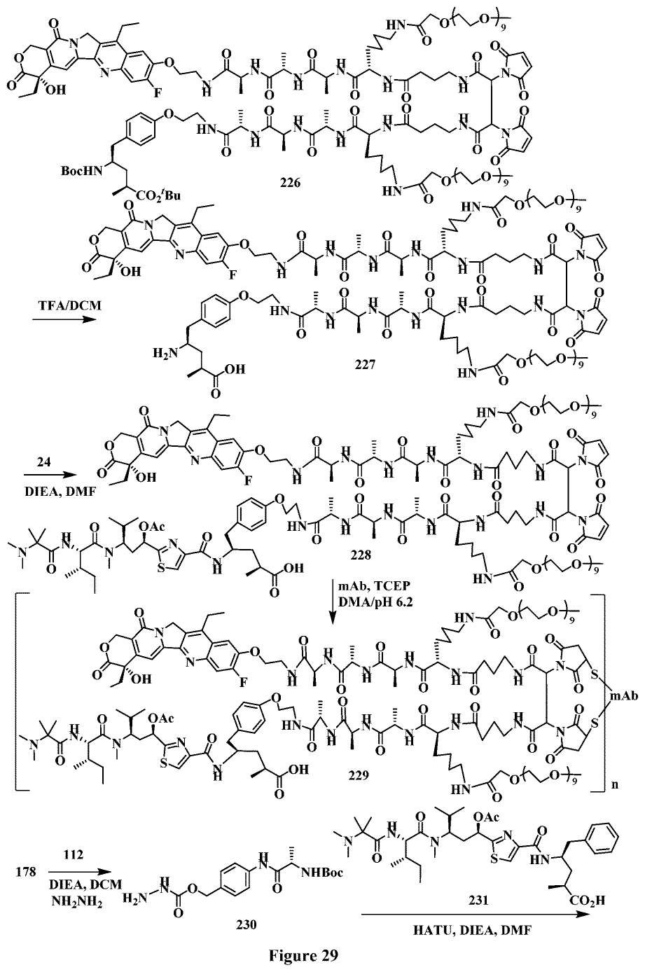

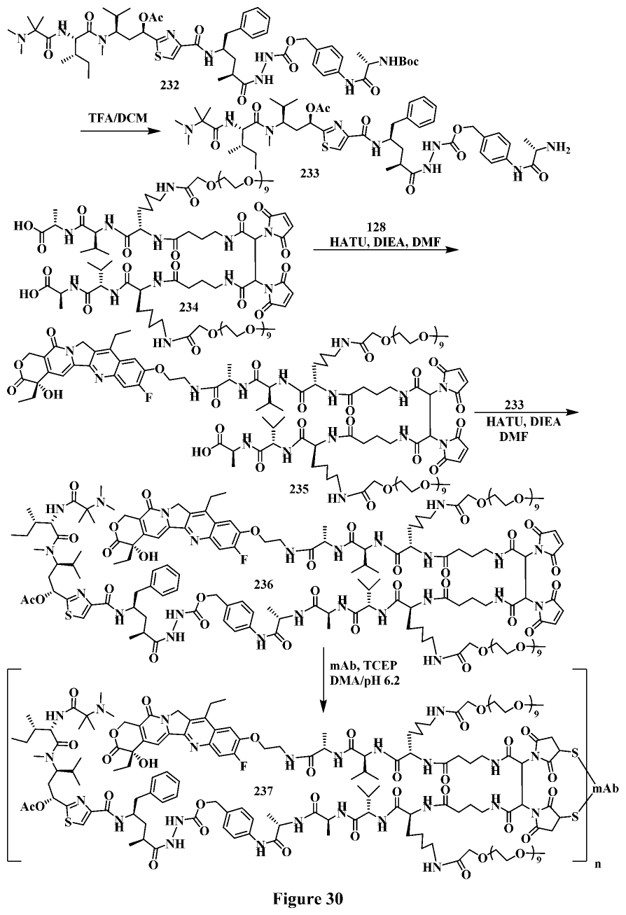

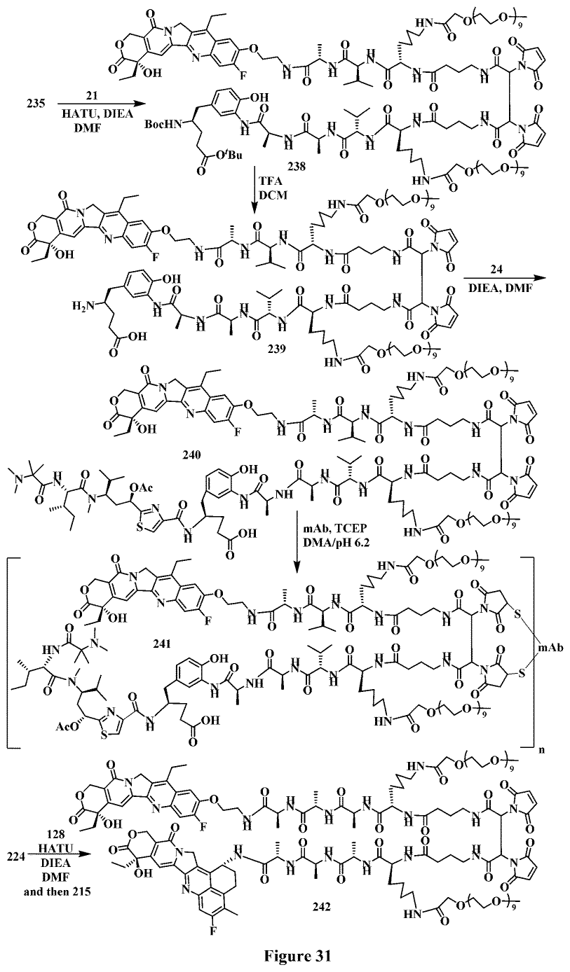

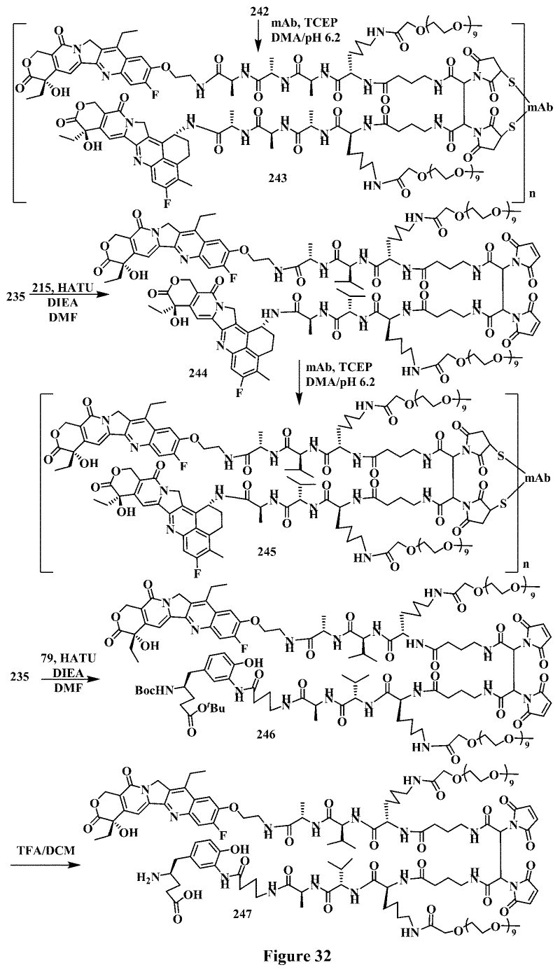

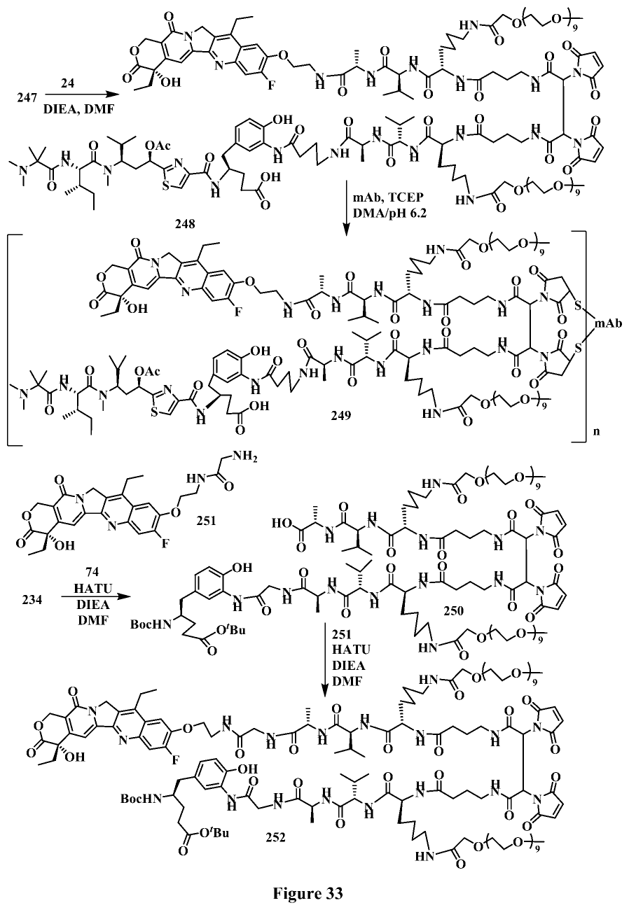

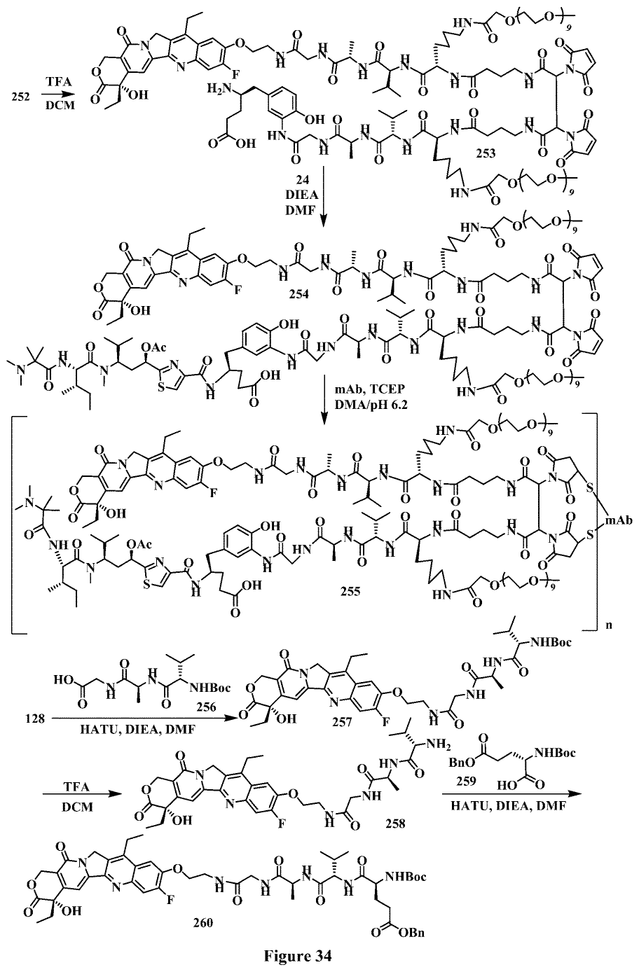

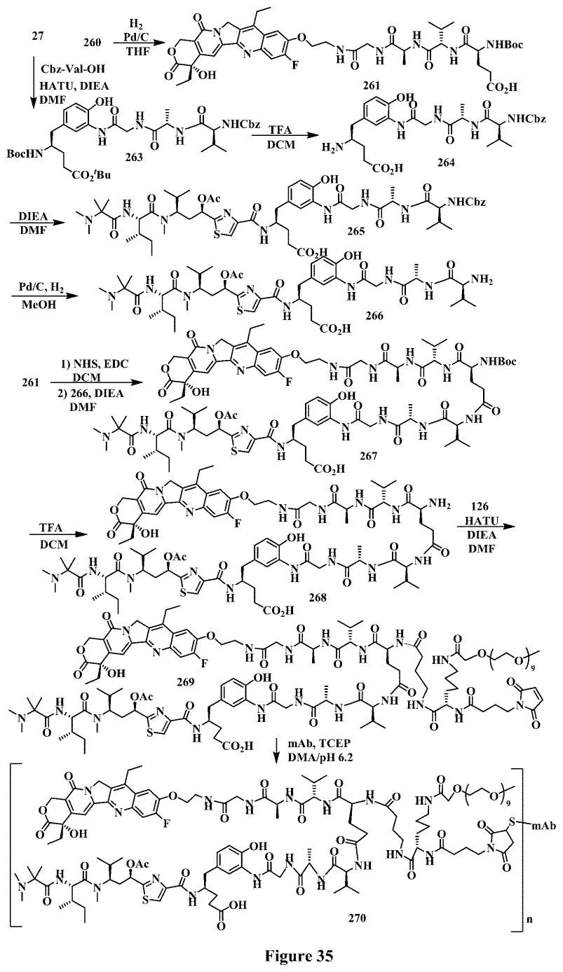

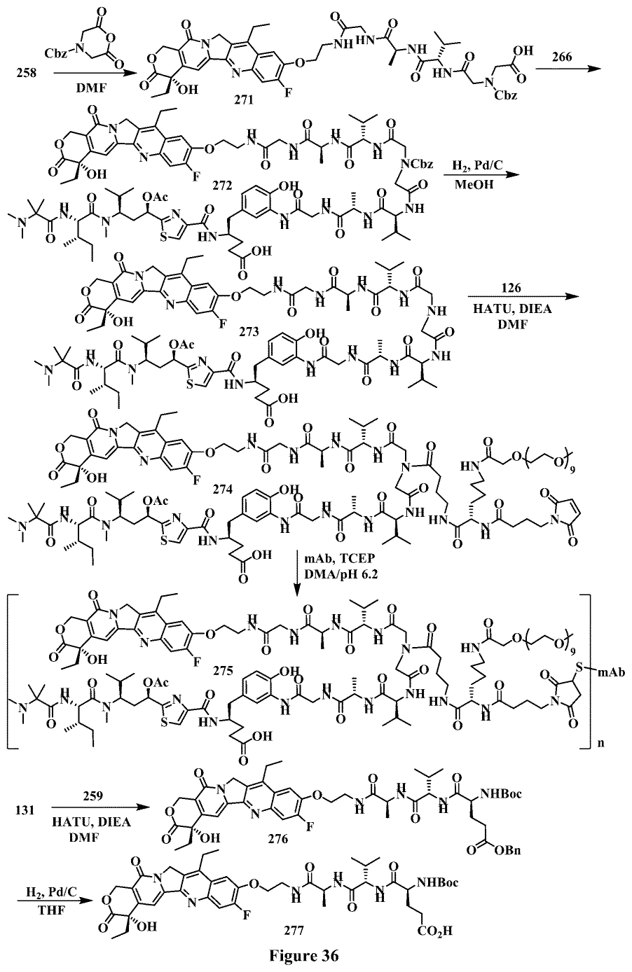

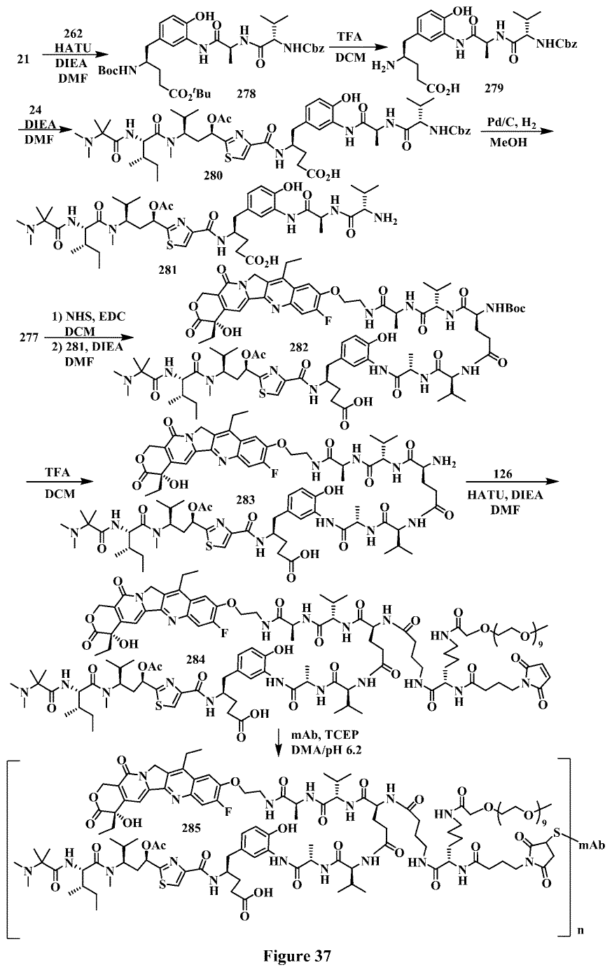

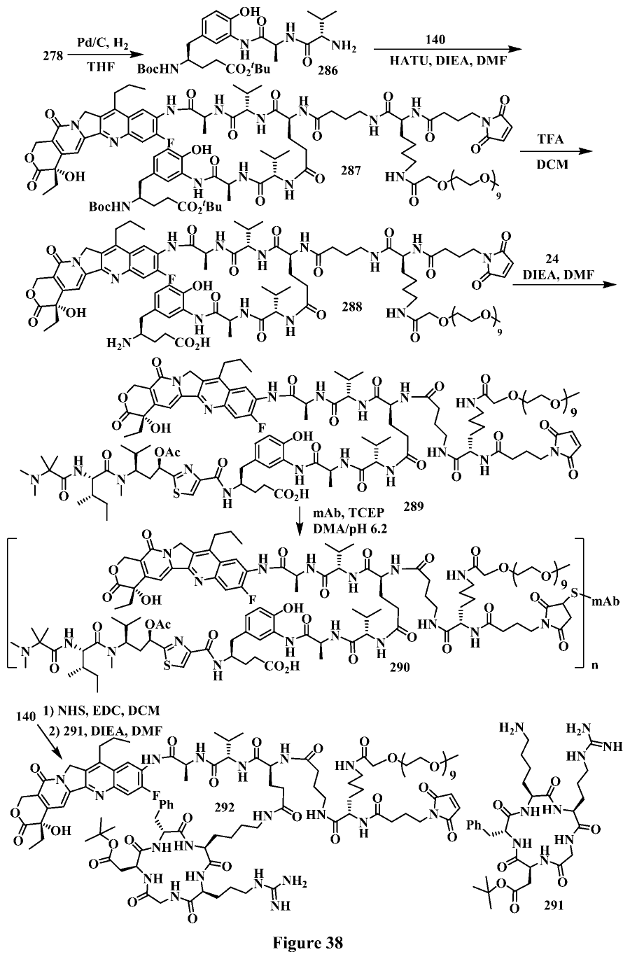

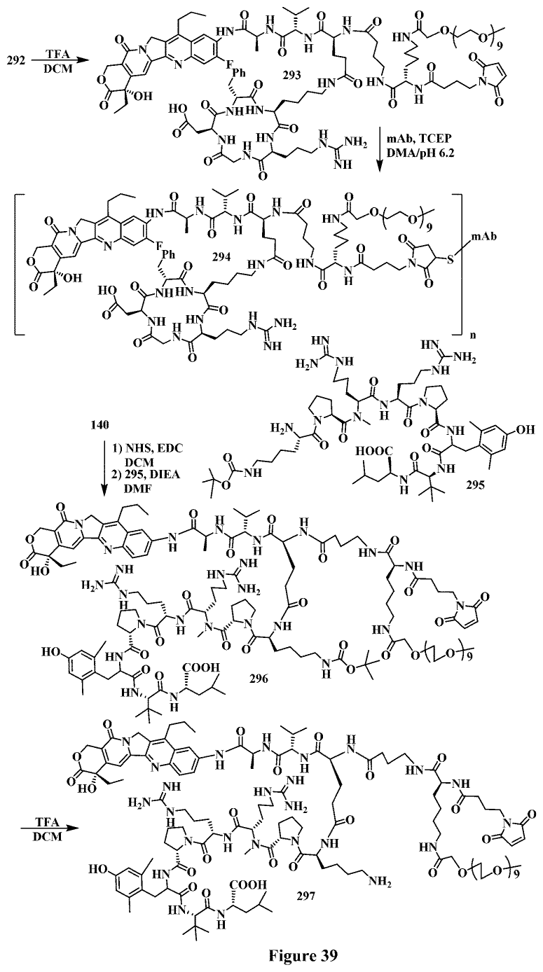

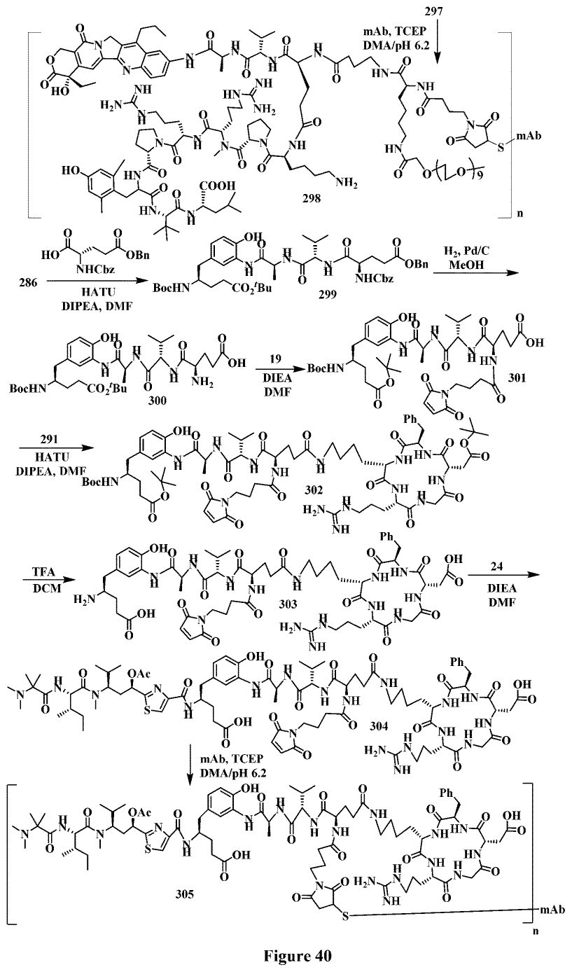

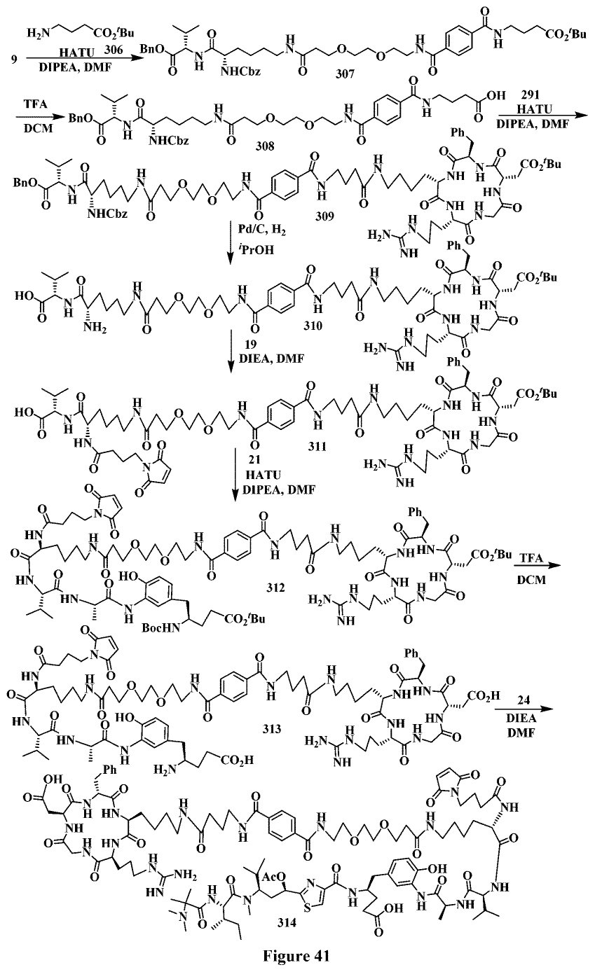

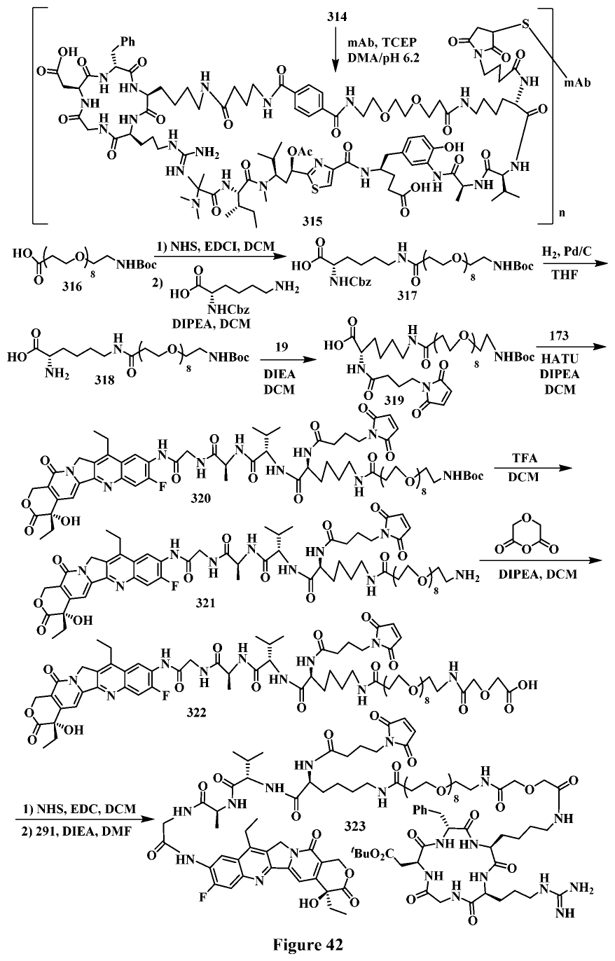

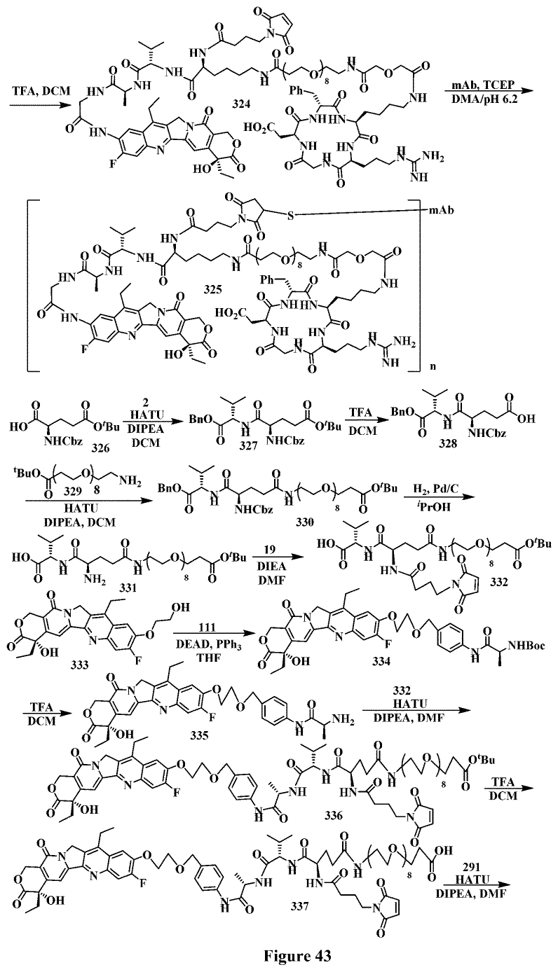

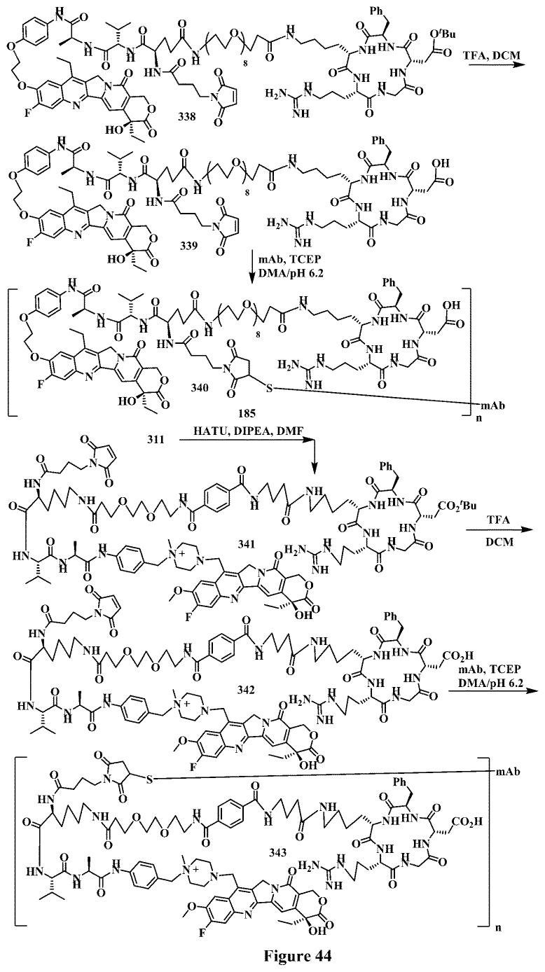

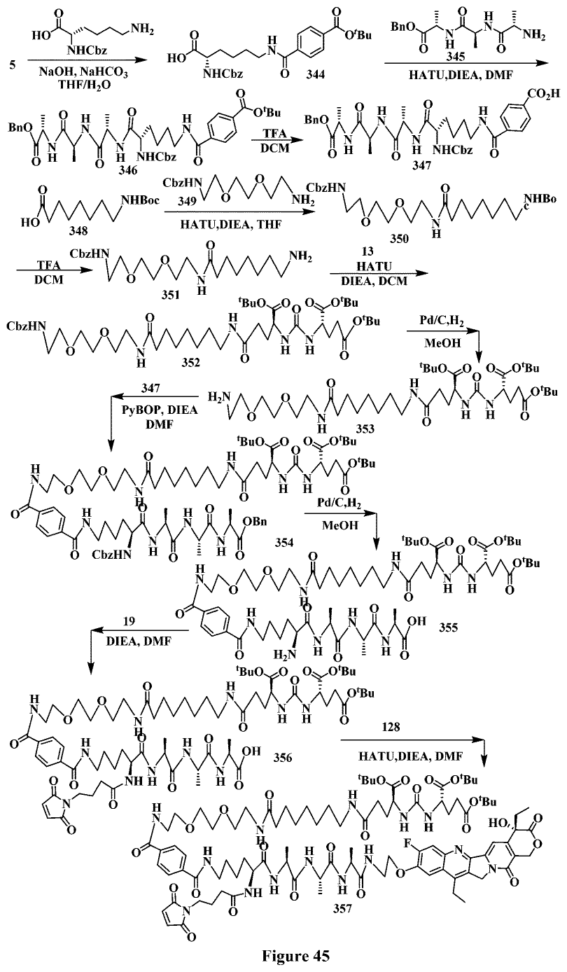

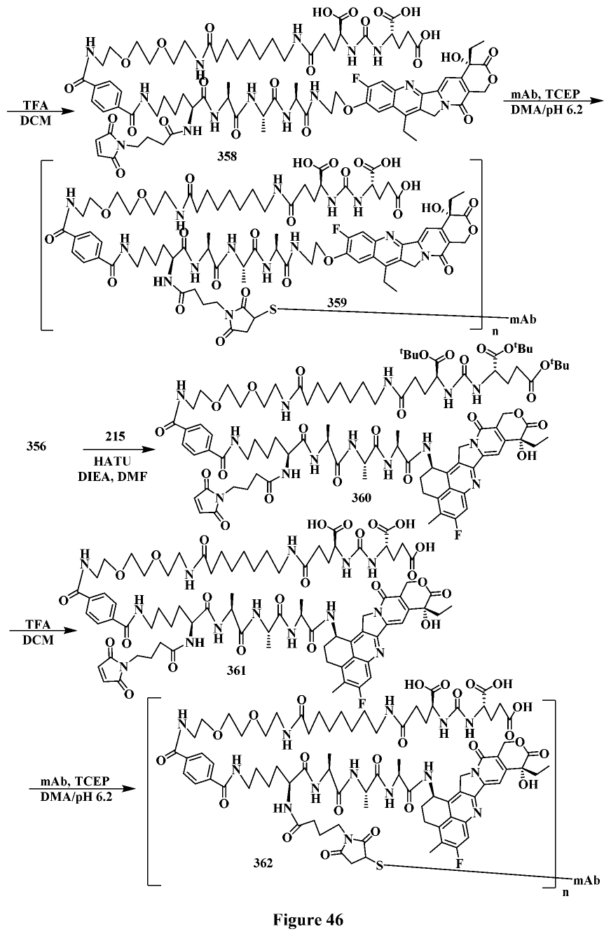

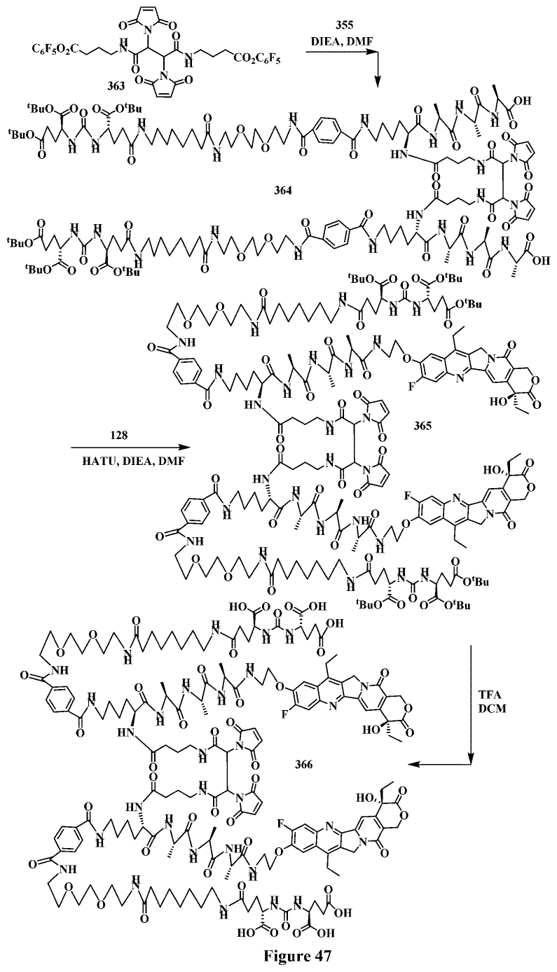

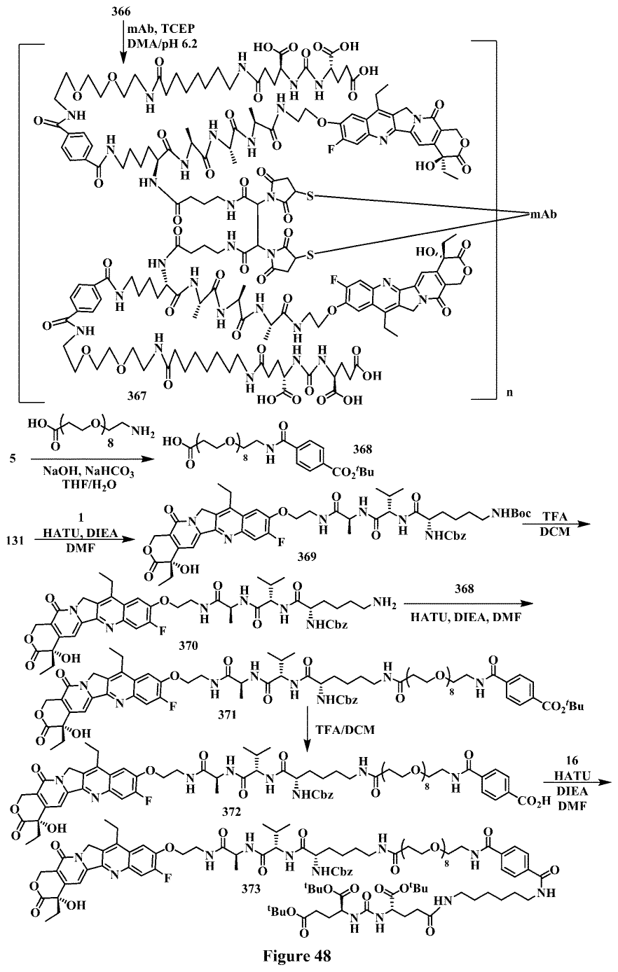

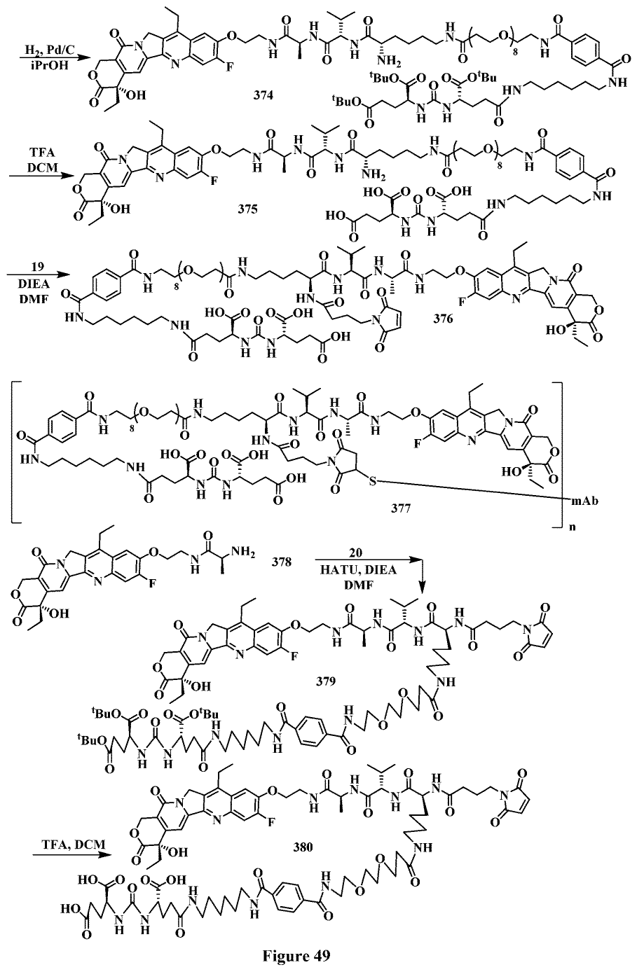

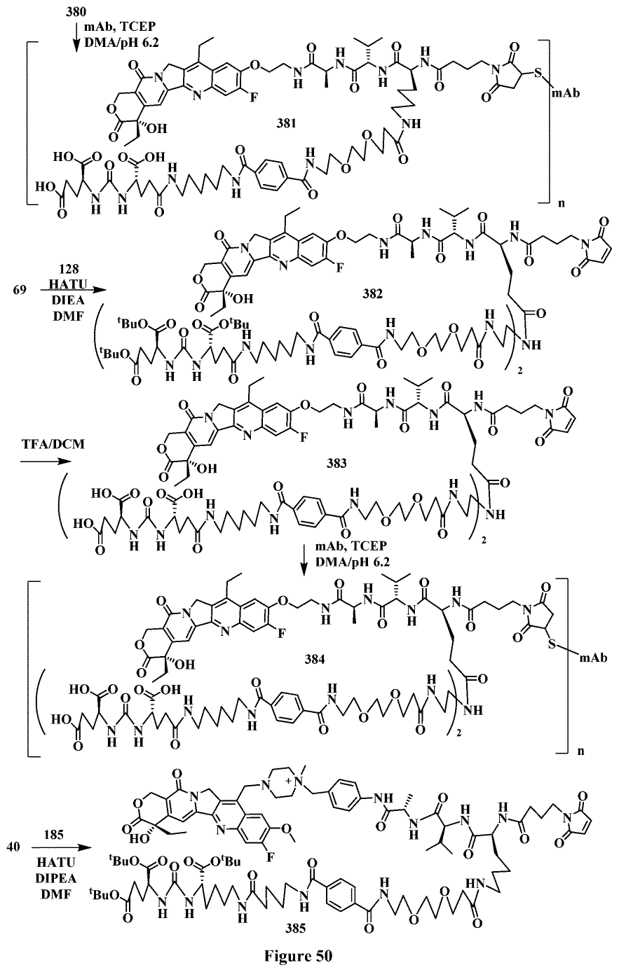

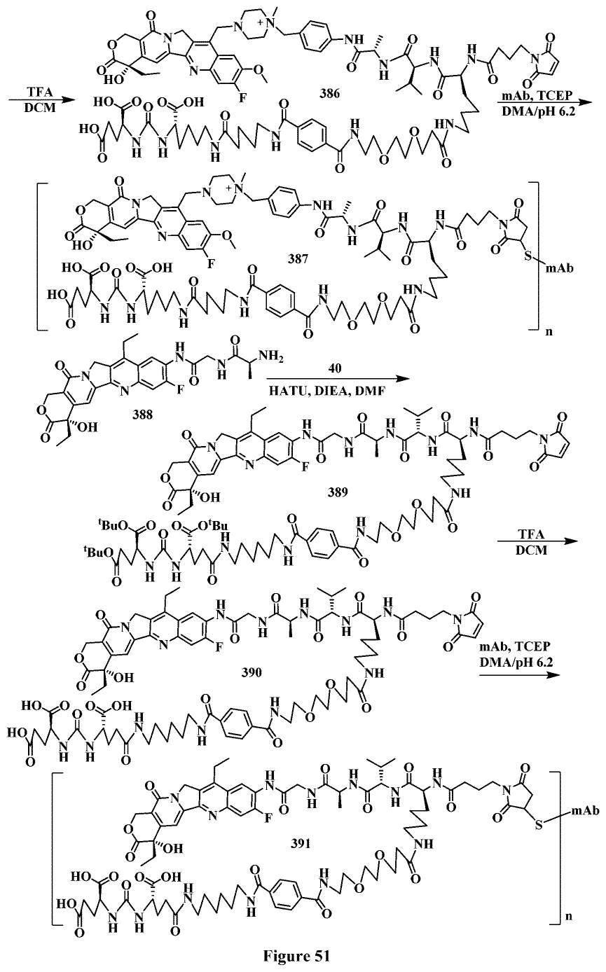

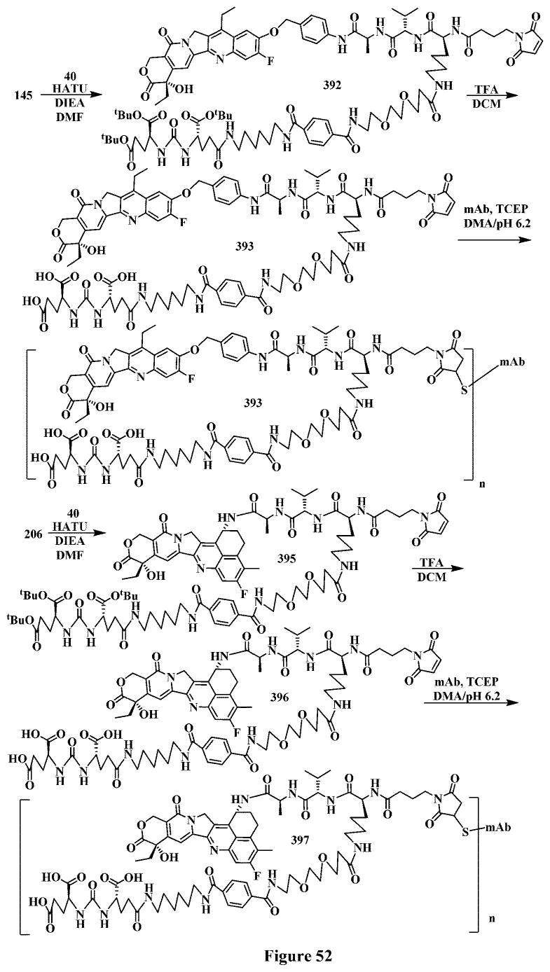

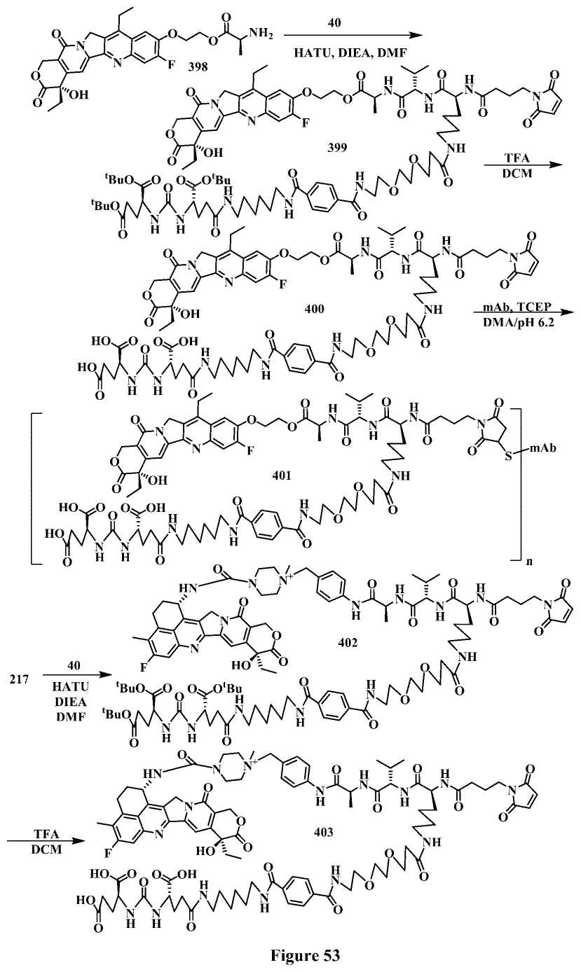

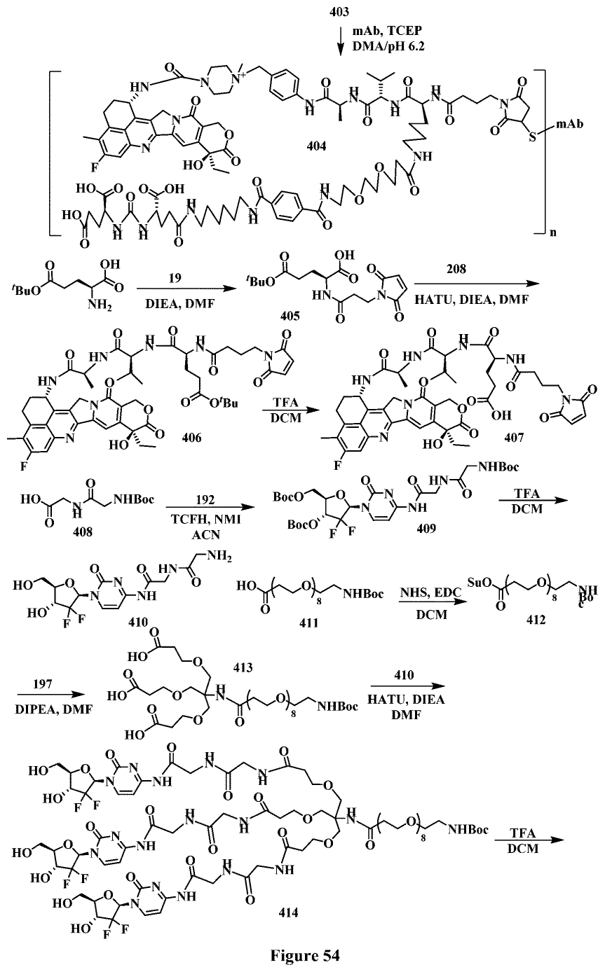

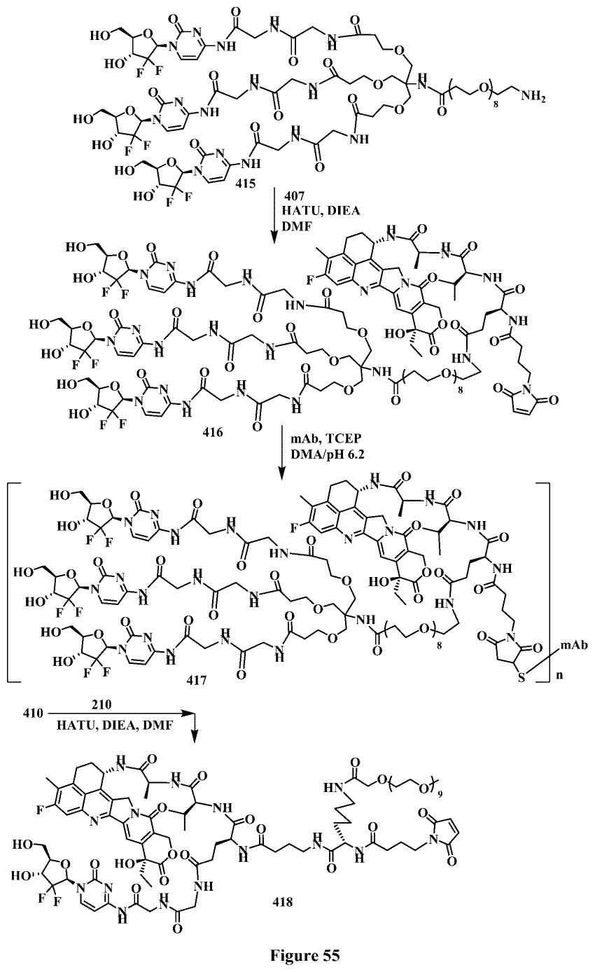

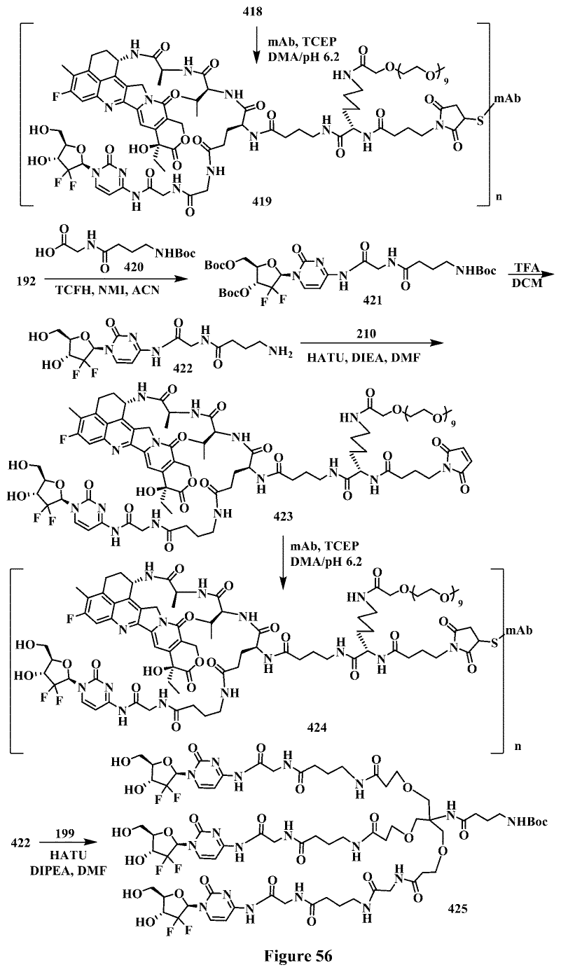

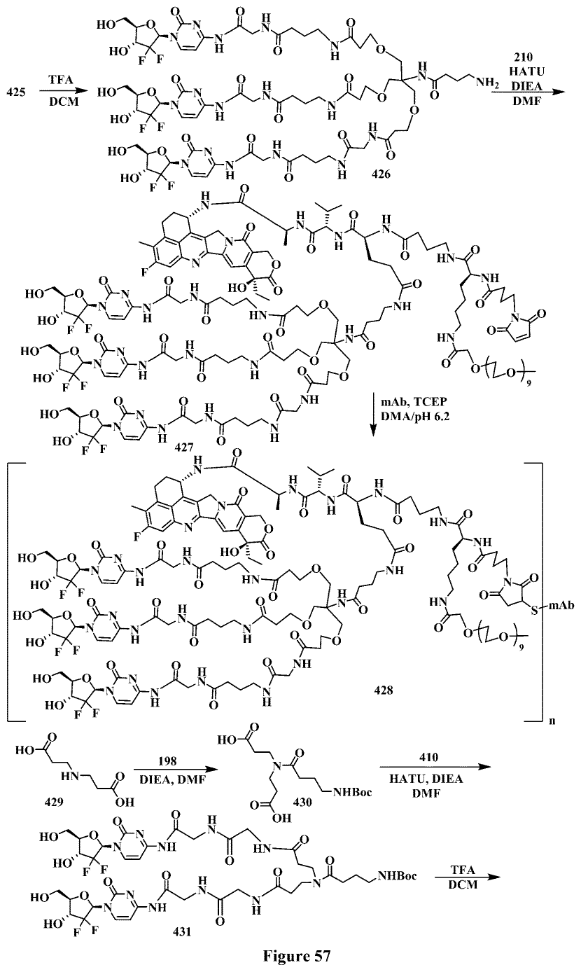

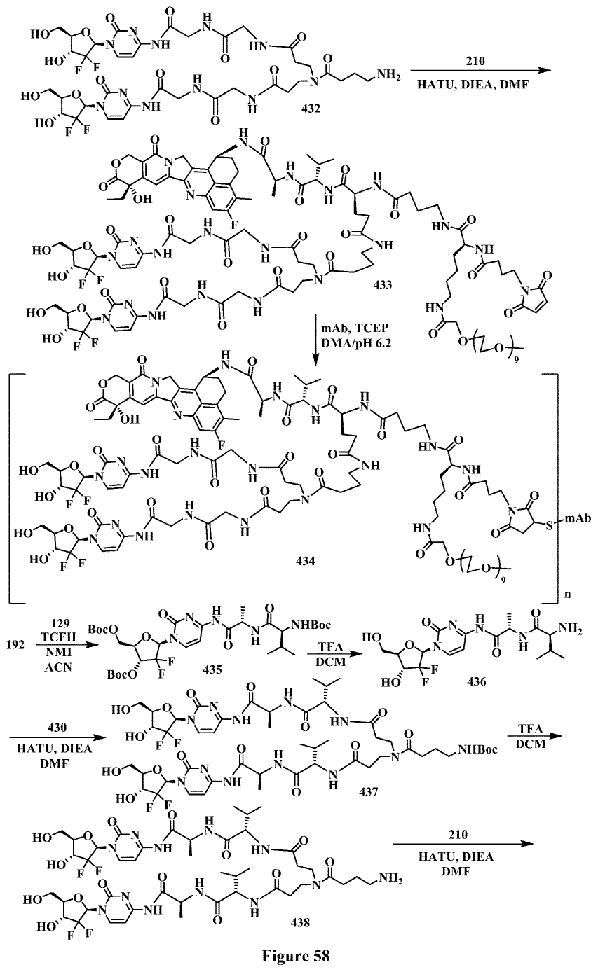

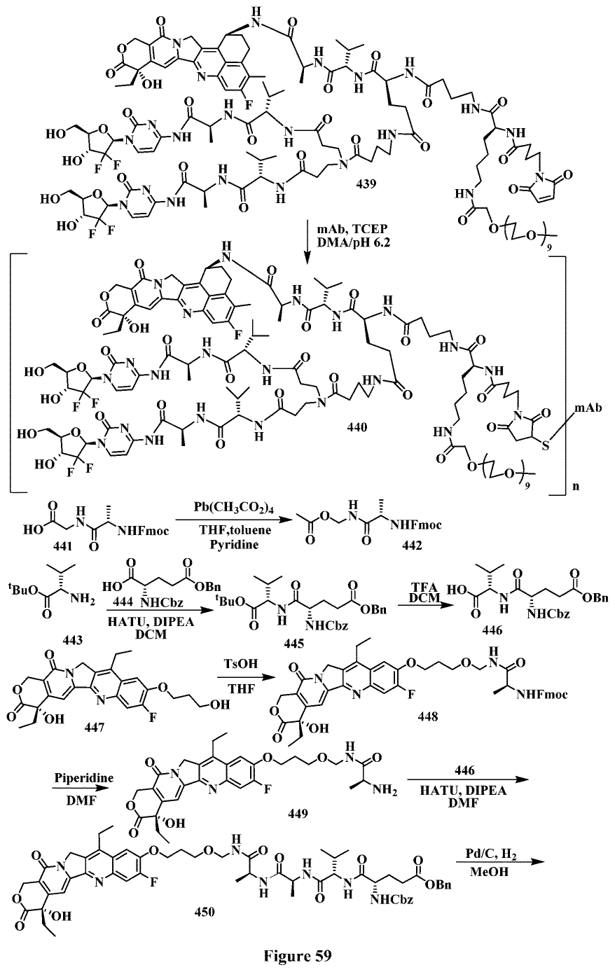

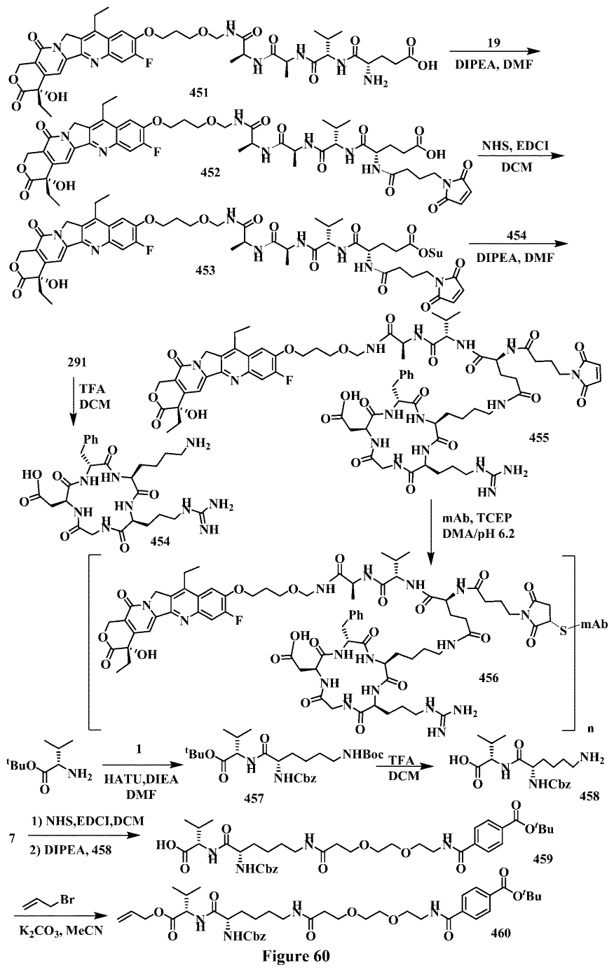

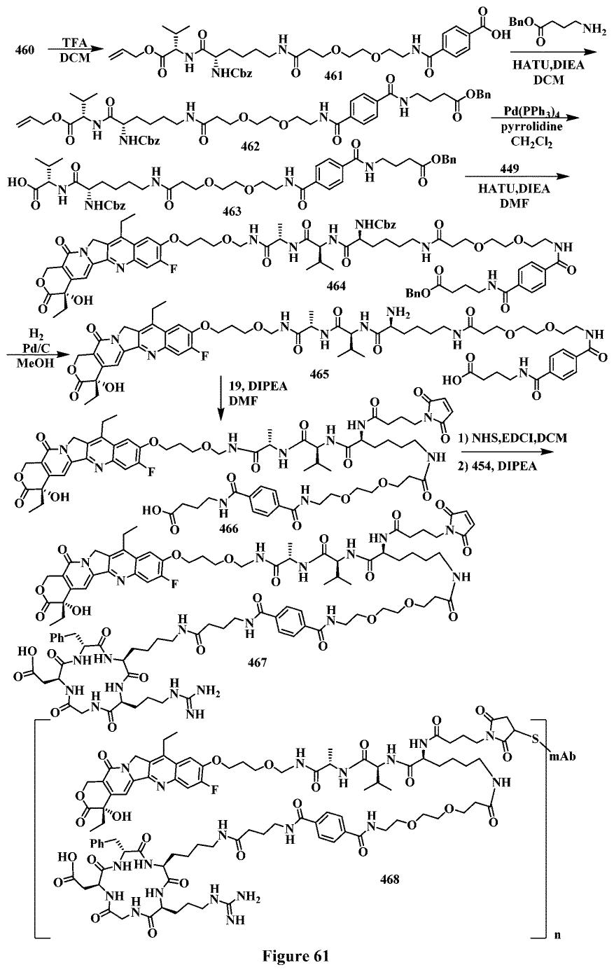

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-Cₛ(1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2, 2-dimethylbutyl, 2, 3-dimethylbutyl, 2, 2-dimethylpentyl, 2, 3-dimethylpentyl, 3, 3-dimethylpentyl, 2, 3, 4-trimethylpentyl, 3-methyl-hexyl, 2, 2-dimethylhexyl, 2, 4-dimethylhexyl, 2, 5-dimethylhexyl, 3, 5-dimethylhexyl, 2, 4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′) 2 and —CN; where each R′ is independently selected from —C₁-C₈ alkyl and aryl. When the alkyl group is inserted in the middle of a group, such as in a middle of a linker, thus the “alkyl” means “alkylene” group in this application.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4, 5], [5, 5], [5, 6] or [6, 6] system, or 9 or 10 ring atoms arranged as a bicycle [5, 6] or [6, 6] system. Representative C₃-C₈ carbocycles include, but are not limited to, -cyclopropyl, -cyclobutyl, -cyclopentyl, -cyclopentadienyl, -cyclohexyl, -cyclohexenyl, -1, 3-cyclohexadienyl, -1, 4-cyclohexadienyl, -cycloheptyl, -1, 3-cycloheptadienyl, -1, 3, 5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′) 2 and —CN; where each R′ is independently selected from —C—C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1, 2-ethyl (—CH₂CH₂—), 1, 3-propyl (—CH₂CH₂CH₂—), 1, 4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1, 2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂0R′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1, 2, 4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate. “Leaving group” refers to a functional group that can be substituted by another functional group.

Such leaving groups are well known in the art, and examples include, a halide (e.g., chloride, bromide, and iodide), methanesulfonyl (mesyl), p-toluenesulfonyl (tosyl), trifluoro-methylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CD1, 1, 1′-carbonyldiimidazole; DCC, dicyclohexylcarbodiimide; DCE, dichloroethane; DCM, dichloromethane; DIAD, diisopropylazodicarboxylate; DIBAL-H, diisobutyl-aluminium hydride; DIPEA, diisopropylethylamine; DEPC, diethyl phosphorocyanidate; DMA, N, N-dimethyl acetamide; DMAP, 4-(N, N-dimethylamino)pyridine; DMF, N, N-dimethylformamide; DMSO, dimethylsulfoxide; DTT, dithiothreitol; EDC, 1-(3-dimethylaminopropyl)-3-ethylcarbodiimide hydrochloride; ESI-MS, electrospray mass spectrometry; HATU, 0-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuccinimide; MMP, 4-methylmorpholine; PAB, p-aminobenzyl; PBS, phosphate-buffered saline (pH 7.0-7.5); PEG, polyethylene glycol; SEC, size-exclusion chromatography; TCEP, tris(2-carboxyethyl)phosphine; TFA, trifluoroacetic acid; THF, tetrahydrofuran; Val, valine.

The “amino acid(s)” can be natural and/or unnatural amino acids, preferably alpha-amino acids. Natural amino acids are those encoded by the genetic code, and their names, structures, single-letter or three letter codes are well known in a college text book as: G - Glycine (Gly), P - Proline (Pro), A -Alanine (Ala), V - Valine (Val), L - Leucine (Leu), I - Isoleucine (Ile), M - Methionine (Met), C -Cysteine (Cys), F - Phenylalanine (Phe), Y - Tyrosine (Tyr), W - Tryptophan (Trp), H - Histidine (His), K - Lysine (Lys), R - Arginine (Arg), Q - Glutamine (Gln), N - Asparagine (Asn), E - Glutamic Acid (Glu), D - Aspartic Acid (Asp), S - Serine (Ser), T - Threonine (Thr). The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3, 4-dihydroxyphenylalanine (DOPA), and 0-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form.

When 1-8 amino acids are used in this patent application, amino acid sequence is then preferably a cleavage recognition sequence for a protease. Many cleavage recognition sequences are known in the art. See, e.g., Matayoshi et al. Science 247:954 (1990); Dunn et al. Meth. Enzymol. 241: 254 (1994); Seidah et al. Meth. Enzymol. 244:175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Val-Ala, Val-Gln, Val-Lys, Tyr-Arg, Phe-Arg, Tyr-Met, Leu-Gln, Met-Thr, Phe-Gln, Thr-Thr, Val-Thr, Ala-Ala, (D)Val-(D)Gln, Val-Lys(NPr₂), Val-Lys(NMe₂), Val-Lys(NEt₂), Val-Lys(NBu₂), Val-Lys(NBz₂), Val-Ala-Val, Lys-Lys, Ala-Asn-Val, Ala-Val-Lys, Ala-Val-Glu, Val-Leu-Lys, Cit-Cit, Val-Lys, Ala-Ala-Asn, Gly-Gly, Gly-Gly-Gly, Ala-Ala-Ala, Ala-Ala-Ala-Glu, Ala-Val-Arg, Ala-Val-Arg-Arg, Ala-Ala-Arg, Ala-Ala-Arg-Arg, Gly-Gly-Phe-Gly, Lys, Cit, Ser, and Glu. Besides 20 standard L- or 20 D- amino acids, some unusual amino acids with letter codes are listed here: Aminobutyric acid (Abu), Amino-isobutyric acid (Aib), α-Cyclohexyl-alanine (Cha), Citrulline (Cit), Diaminopropionic acid (Dap), Hydroxy-lysine (Hyl), Hydroxy-proline (Hyp), Norleucine (NIe), Norvaline (Nva), Ornithine (O), Penicilamine (Pen), Pyroglutamate (Pyr), Sarcosine (Sar), Statine (Sta). Modified amino acids with single codes have the following examples: Asparagine-EDANS (D-EDANS), Cysteine 3-Nitro-2-pyridinesulfanyl (C-NPys), Glutamic acid-EDANS (E-EDANS), Glycine N-methylated (G-NMe), Leucine N-methylated (L-NMe), Serine phosphorylated (pS), Threonine phosphorylated (pT), Tyrosine phosphorylated (pY), Tyrosine O-methylated (Y—OMe), 3-Nitrotyrosine (Y—NO2), Tyrosine sulphated (sY), Lysine 5-Carboxyfluorescein (K-5-FAM), Lysine 5-Carboxytetramethylrhodamine (K-5-TAMRA), Lysine acetylated (K—Ac), Lysine biotinylated (K-Biotin), Lysine-DABCYL (K-DABCYL), Lysine-DANSYL (K-DANSYL), Lysine-Dnp (K-Dnp), Lysine-Mca (K-Mca), Lysine methylated (K-Me), Lysine dimethylated (K-Me2), Lysine trimethylated (K-Me3).

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two.

Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, PA, 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

The abbreviations of biological buffers and their chemical names are listed below: ACES (N-(2-Acetamido)-2-aminoethanesulfonic acid) is used to buffer at pH 6.1-7.5 (pKa=6.88)
ADA (N-(2-Acetamido)iminodiacetic acid, N-(Carbamoylmethyl)iminodiacetic acid) is useful to buffer at pH 6.0-7.2 (pKa=6.65).
AMPD (2-amino-2-methyl-1, 3-propanediol)) is a useful buffer at pH 7.8-9.7.
AMPSO (N-(1, 1-Dimethyl-2-hydroxyethyl)-3-amino-2-hydroxypropanesulfonic acid).
BES (N, N-Bis(2-hydroxyethyl)-2-aminoethanesulfonic acid).
Bicine (N, N-Bis(2-hydroxyethyl)glycine], Bis(2-hydroxyethyl)amino-tris(hydroxymethyl) methane) is used to buffer at pH 5.8-7.2 (pKa=8.35).
BisTris (Bis-(2-Hydroxyethyl)amino-tris(Hydroxymethyl)Methane).
BisTris propane (1, 3-Bis[tris(hydroxymethyl)methylamino]propane).
DIPSO (N, N-Bis(2-hydroxyethyl)-3-amino-2-hydroxypropanesulfonic acid) is used to buffer at pH 7.0-8.2.
Gly-Gly (Diglycine; Glycyl-glycine) is used to buffer at pH 7.5-8.9 (pKa=8.30).
HEBPS (N-(2-Hydroxyethyl)piperazine-N′-(4-butanesulfonic acid)) is a homolog of HEPE^{I} and EPPS with higher pKa (pKa=8.30), used to buffer at pH 7.6-9.0
HEPES (4-(2-hydroxyethyl)-1-piperazineethanesulfonic acid; 2-morpholinoethanesulfonic acid; 2-(4-morpholino)ethanesulphonic acid; 2-(N-morpholino)ethanesulfonic acid; morpholine-4-ethanesulfonic acid hydrate) is widely used to buffer at pH 6.8-8.2; pKa at 20° C.: 7.45-7.65)
HEPPS or EPPS (3-[4-(2-Hydroxyethyl)-1-piperazinyl]propanesulfonic acid hydrate; 4-(2-Hydroxyethyl)piperazine-1-(2-hydroxypropanesulfonic acid) Hydrate) is used as a buffering agent at pH 7.3-8.7 (pKa=8.00/piperazine ring).
HEPPSO (4-(2-Hydroxyethyl)piperazine-1-(2-hydroxypropanesulfonic acid) hydrate).
MES (2-(N-morpholino)ethanesulfonic acid, monohydrate) is used as buffering agent at pH 5.2-7.1 (pKa:6.16).
MOBS (4-Morpholinebutanesulfonic acid; 3-(N-Morpholino)butanesulfonic acid hemisodium salt) is a homolog of MES and MOPS with higher pKa/It is used to buffer solution at pH6.9-8.3 (pKa:7.6).
MOPS (4-Morpholinepropanesulfonic acid sodium salt).
MOPSO ($-Hydroxy-4-morpholinepropanesulfonic acid, 3-Morpholino-2-hydroxypropanesulfonic acid).
PIPES (Piperazine-1, 4-bis(2-ethanesulfonic acid) is used to buffer at pH 6.1-7.5 (pKa=6.80).
POPSO (Piperazine-1, 4-bis(2-hydroxypropanesulfonic acid) dihydrate).
TAPS ([(2-Hydroxy-1, 1-bis(hydroxymethyl)ethyl)amino]-1-propanesulfonic acid).
TAPSO (2-Hydroxy-3-[tris(hydroxymethyl)methylamino]-1-propanesulfonic acid).
TES (2-[(2-Hydroxy-1, 1-bis(hydroxymethyl)ethyl)amino]ethanesulfonic acid).
Tricine (Piperazine-N, N′-Bis[2-Hydroxypropanesulfonic Acid)] is used to buffer at pH7.4-8.8 (pKa:8.16).

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′—SH, F(ab′)₂; diabodies; linear antibodies; single-chain antibody molecules (e.g. scFv); and multispecific antibodies formed from antibody fragments. A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDR₈) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FR₈) and three hypervariable regions (HVR₈). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150: 880-887 (1993); Clarkson et al., Nature 352: 624-628 (1991).

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495, 1975, or may be made by recombinant DNA methods such as described in U.S. Pat. No. 4,816,567. The monoclonal antibodies may also be isolated from phage libraries generated using the techniques described in McCafferty et al., Nature 348:552-554, 1990, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen binding subsequences of antibodies) that contain minimal sequence derived from non-human immunoglobulin. Preferably, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L₂, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5, 545, 806; 5, 569, 825; 5, 625, 126; 5, 633, 425; and 5, 661, 016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “monovalent antibody” comprises one antigen binding site per molecule (e.g., IgG or Fab). In some instances, a monovalent antibody can have more than one antigen binding sites, but the binding sites are from different antigens.

A “monospecific antibody” comprises two identical antigen binding sites per molecule (e.g. IgG) such that the two binding sites bind identical epitope on the antigen. Thus, they compete with each other on binding to one antigen molecule. Most antibodies found in nature are monospecific. In some instances, a monospecific antibody can also be a monovalent antibody (e.g. Fab).

A “bivalent antibody” comprises two antigen binding sites per molecule (e.g., IgG). In some instances, the two binding sites have the same antigen specificities. However, bivalent antibodies may be bispecific.

A “bispecific” or “dual-specific” is a hybrid antibody having two different antigen binding sites. The two antigen binding sites of a bispecific antibody bind to two different epitopes, which may reside on the same or different protein targets.

A “bifunctional” is antibody is an antibody having identical antigen binding sites (i.e., identical amino acid sequences) in the two arms but each binding site can recognize two different antigens.

A “heteromultimer”, “heteromultimeric complex”, or “heteromultimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present.

A “heterodimer”, “heterodimeric protein”, “heterodimeric complex,” or “heteromultimeric polypeptide” is a molecule comprising a first polypeptide and a second polypeptide, wherein the second polypeptide differs in amino acid sequence from the first polypeptide by at least one amino acid residue.

The “hinge region”, “hinge sequence”, and variations thereof, as used herein, includes the meaning known in the art, which is illustrated in, for example, Janeway et al., ImmunoBiology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

The “immunoglobulin-like hinge region”, “immunoglobulin-like hinge sequence,” and variations thereof, as used herein, refer to the hinge region and hinge sequence of an immunoglobulin-like or an antibody-like molecule (e.g., immunoadhesins). In some embodiments, the immunoglobulin-like hinge region can be from or derived from any IgG1, IgG2, IgG3, or IgG4 subtype, or from IgA, IgE, IgD or IgM, including chimeric forms thereof, e.g., a chimeric IgG1/2 hinge region.

Since the disulfide bonds in different IgG forms of antibody are various, thus drug/antibody ratios (DAR₈) with the thiol-ether conjugation (such as through the Michael addition reaction of a maleimide of a drug/linker complex and a cysteine in an antibody) can be various. For instance, the DARs (or “n” in this application) can be up to 30 for IgG2, IgG3 or IgG4 form of an ADC.

The term “immune effector cell” or “effector cell” as used herein refers to a cell within the natural repertoire of cells in the human immune system which can be activated to affect the viability of a target cell. The viability of a target cell can include cell survival, proliferation, and/or ability to interact with other cells.

Antibodies of the invention can be produced using techniques well known in the art, e.g., recombinant technologies, phage display technologies, synthetic technologies or combinations of such technologies or other technologies readily known in the art (see, for example, Jayasena, S. D., Clin.

Chem., 45:1628-50, 1999 and Fellouse, F. A., et al, J. Mol. Biol., 373(4): 924-40, 2007).

The term “cytotoxic agent” as used herein refers to a substance that inhibits or prevents a cellular function and/or causes cell death or destruction. Cytotoxic agents include, but are not limited to, radioactive isotopes (e.g., At211, 1131, 1125, Y90, In111, Re186, Re188, Sm153, Bi212, P32, Pb212, Zr89, F18, and radioactive isotopes of Lu, e.g. Lu177); chemotherapeutic agents or drugs (e.g., tubulysin, maytansin, auristatin, DNA minor groove binders (such as PBD dimers), duocarmycin, topoisomerase inhibitor I or II (such as camptothecins or etoposides), RNA polymerase inhibitors, DNA alkylators, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed throughout the application. “Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)nO(CR₂)n—, repeating units of alkyloxy (e.g. polyethylenoxy, PEG, polymethyleneoxy) and alkylamino (e.g. polyethyleneamino); and diacid ester and amides including succinate, succinamide, diglycolate, malonate, and caproamide. In various embodiments, linkers can comprise one or more amino acid residues, such as valine, phenylalanine, lysine, and homolysine.

The words “comprise”, “comprising”, “include”, “including” and “includes” when used in this specification and claims are intended to specify the presence of stated features, integers, components, or steps, but they do not preclude the presence or addition of one or more other features, integers, components, steps, or groups thereof. The novel conjugates disclosed herein are the antibody conjugates targeting tumor specific antigens. Examples of the conjugates and their synthesis are shown in the examples 1-482 below of the specification.

### The Antibody Drug Conjugate of the Invention.

The invention provides an antibody-drug conjugate that have enhancement of killing of tumor cells. The antibody-drug conjugate (ADC) contains a branch (side chain) linker wherein a group of an affinity small molecule, an affinity peptide or/and a cell penetrating peptide, or a small molecule chemotherapeutic drug at the terminal of the side chain linker that have synergies of cytotoxicity and affinity with the antibody, resulting in enhanced treatment of the tumors and refractory diseases. The formulas of the ADC of the present invention are represented as:

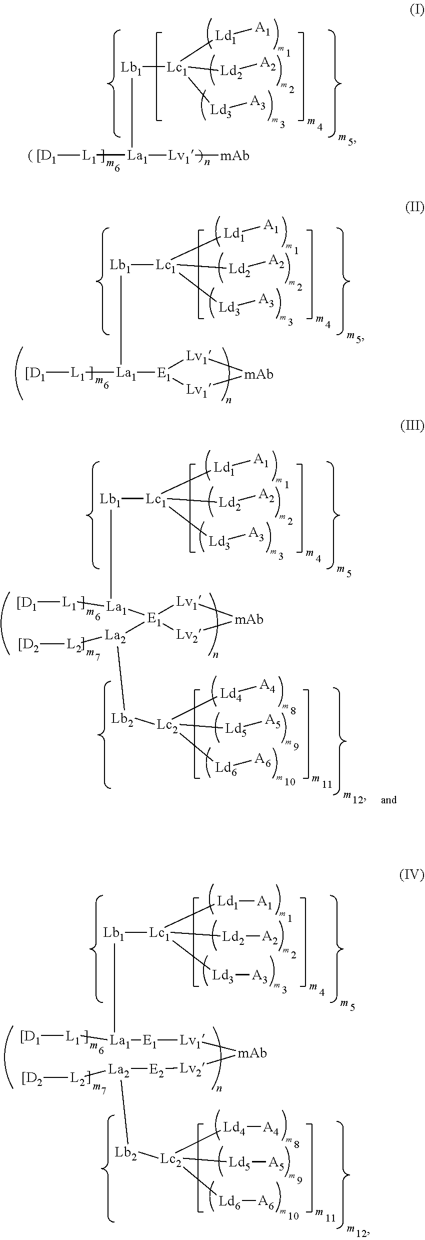

### Wherein,

D₁ and D₂ are a cytotoxic agent; mAb is an antibody or antibody like protein; n is 1, 2, 3, 4, 5, 6, 7, 8, 9, 10, 11, 12, 13, 14, 15, 16, 17, 18, 19 or 20, and these numbers may have a decimal;
L₁, L₂, La₁, La₂, Lb₁, Lb₂, Lc₁, Lc₂, Ld₁, Ld₂, Ld₃, Ld₄, Ld₅, and Ld₆ are a linker component, independently selected from O, NH, S, N, NH—NH, N—N, N(R₃), N(R₃)N(R_{3′}), C(═O)N, C(═O)NH, C(═O)N—N, C₁-Cₛ of alkyl; C₂-Cₛ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, urea, carbamate, carbonate, thiourea, thioether, thiourea, amide, aminoalkylcarboxyl or oxylalkylcarboxyl; or 1~8 natural or unnatural amino acids described in the definition; or polyethyleneoxy unit of formula (OCH₂CH₂)ₚOR₃, C(O)(CH₂CH₂O)ₚR₃, (OCH₂CH(CH₃))ₚOR₃, or NH(CH₂CH₂O)ₚR₃, or NH(CH₂CH(CH₃)O)ₚR₃, or N[(CH₂CH₂O)ₚR₃][(CH₂CH₂O)ₚ>R₃′], or (OCH₂CH₂)ₚCOOR₃, or CH₂CH₂(OCH₂CH₂)ₚCOOR₃, wherein p and p′ are independently an integer selected from 0 to about 100, or combination thereof; wherein R₃ and R₃′ are independently H, C(═O)H, C(═O)CH₃, C₁-Cₛ of alkyl; or combination above thereof;

In further embodiments, L₁, L₂, La₁, La₂, Lb₁, Lb₂, Le₁, Lc₂, Ld₁, Ld₂, Ld₃, Ld₄, Ld₅, and Ld₆ are 1~4 natural or unnatural amino acids, C₁-C₈ of gamma amino-alkyl, aminoalkylcarboxyl, thioalkylcarboxyl or oxylalkylcarboxyl, or C₃-C₁₅ of gamma amino-benzyl or aromatic of ester, ether, urea, carbamate, carbonate, thiourea, thioether, thiourea, or amide, wherein aromatic is selected from phenyl, phenol, benzyloxyl, benzyloxyl, benzylamino, phenylcarboxyl, oxyl-phenyl-carboxyl, amino-phenyl-carboxyl, benzyloxycarbonyl, benzylaminocarbonyl, triazol, tetrazol, pyridinyl, pyrimidinyl, pyrazineyl, oxazole, thiazole. In addition, lysine and or glutamic acid among natural or unnatural amino acids can have a side chain of a polyethyleneoxy unit of formula C(O)(CH₂CH₂O)ₚR₃, or NH(CH₂CH₂O)ₚR₃ linked to the amino or carboxy group of lysine and or glutamic acid respectively, wherein p and R₃ are described above;
E₁ and E₂ are a joint group selected from CH, CH₂, CH—CH, NH, NHNH, N(R₃), N(R₃)N(R_{3′}), N═N, N—N, P, P(═O), S, Si, C₂-Cₛ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing 1-4 units of aminoacids, preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine; or one of the following structures:

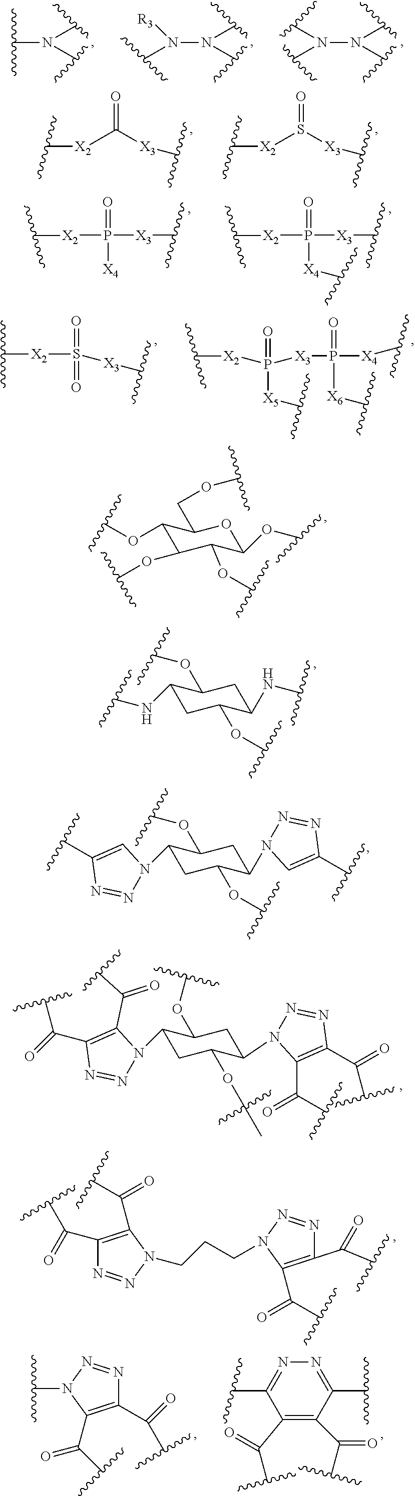

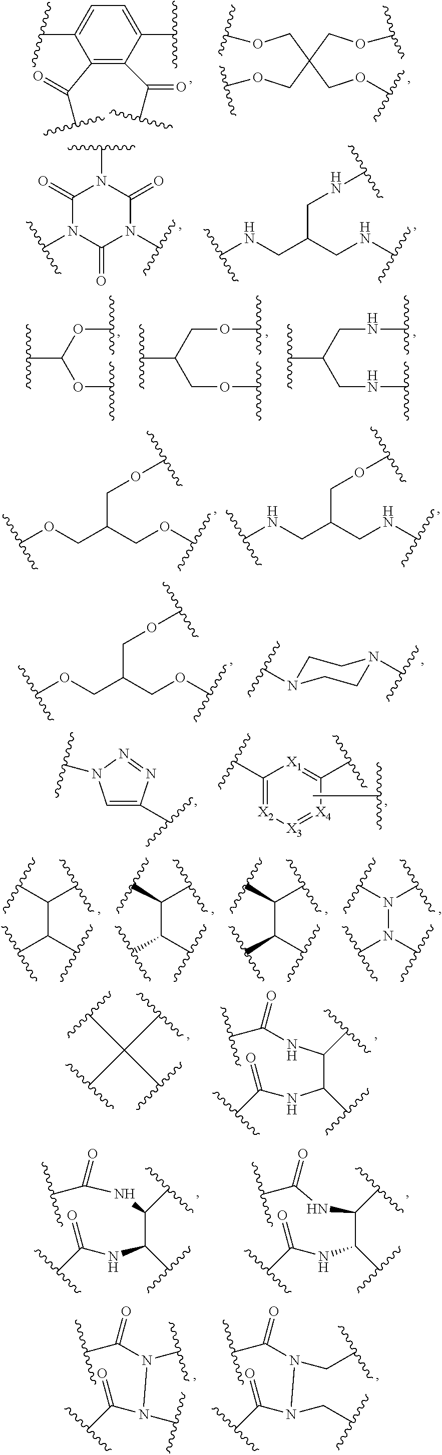

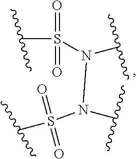

wherein [symbol] is the site of linkage; X₁, X₂, X₃, X₄, X₅, or X₆, are independently selected from NH; NHNH; N(R₃); N(R₃)N(R_{3′}); O; S; C₁-C₆ of alkyl; R₃ and R₃′ are H, C₁-C₆ of alkyl; In addition, E₁ can be absent, thus La₁ or/and La₂ can directly link to Lv₁′ or Lv₂′;
m₁, m₂, m₃, m₄, m₅, m₆, m₇, m₈, m₉,m₁₀, m₁₁ and m₁₂ are independently 1, 2, 3, 4, 5, 6, 7, 8, 9, 10, and these numbers may have a decimal; in addition, m₂, m₃, m₈, m₉ and/or m₁₀ can be 0, thus Ld₂ A₂, Ld₃ A3, Ld₅ A5, and/or Ld₆ A₆ can be absent;
wherein Lv₁′ and Lv₂′ are independently or jointly having the following structures:

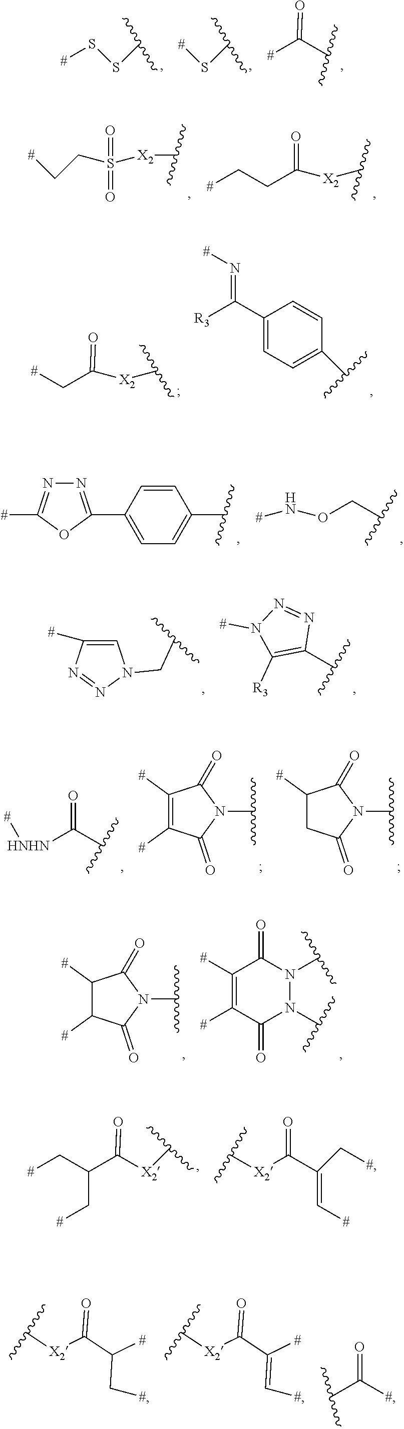

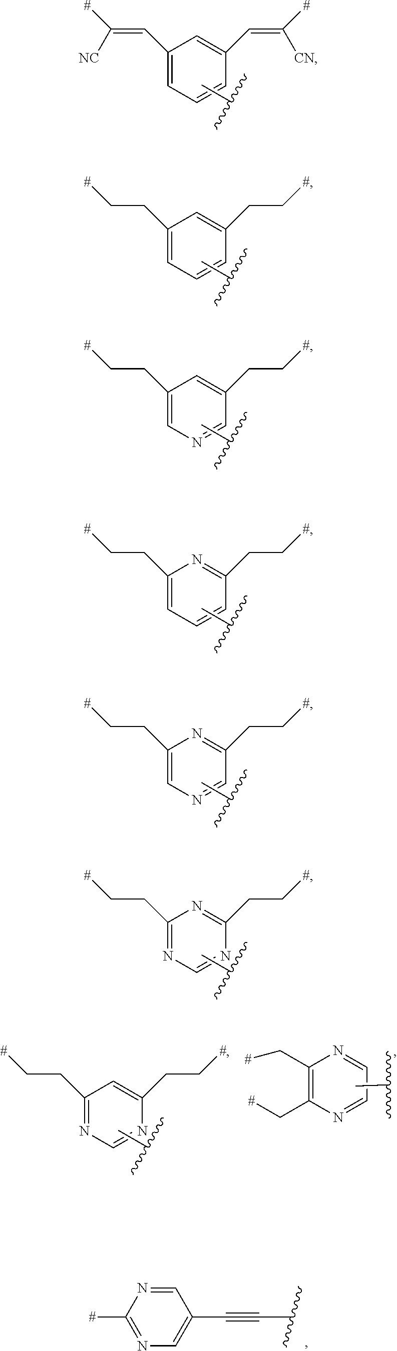

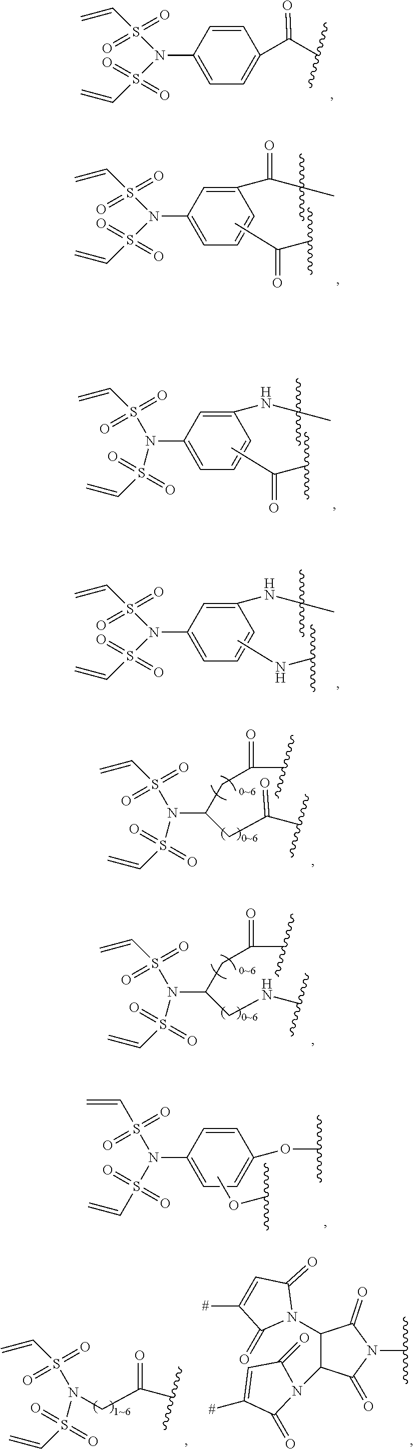

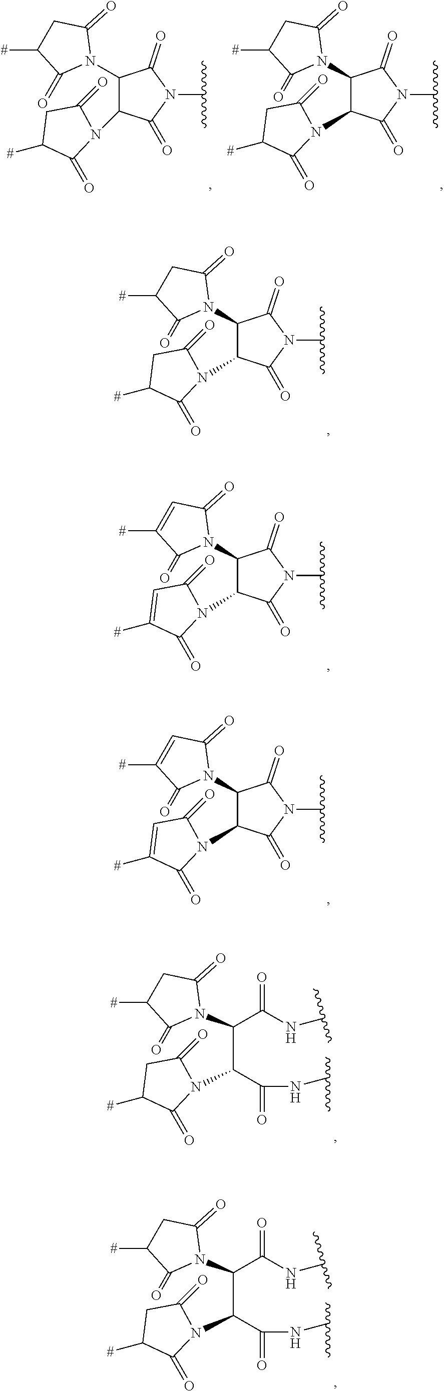

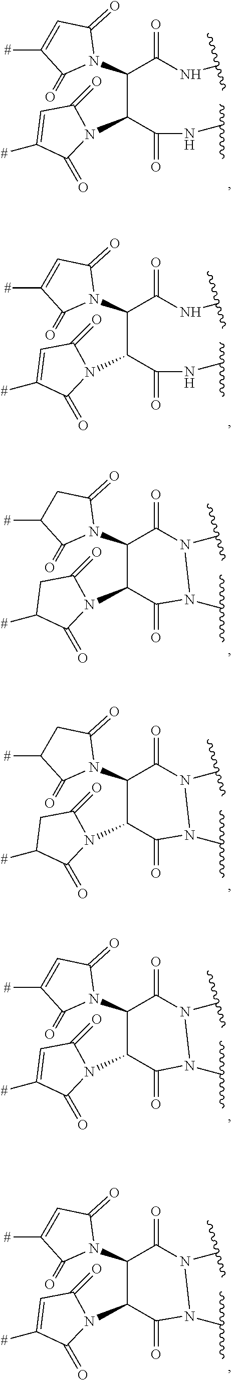

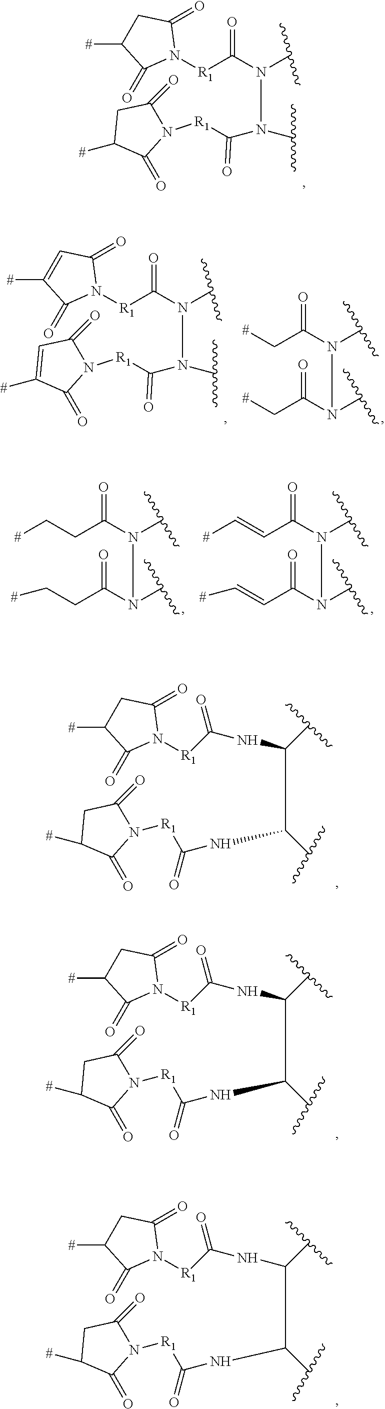

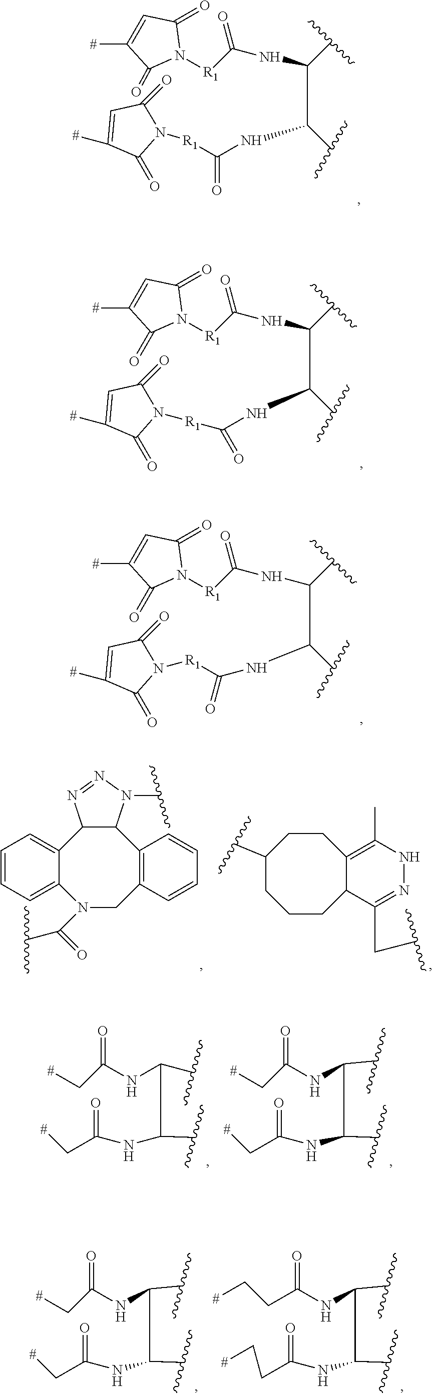

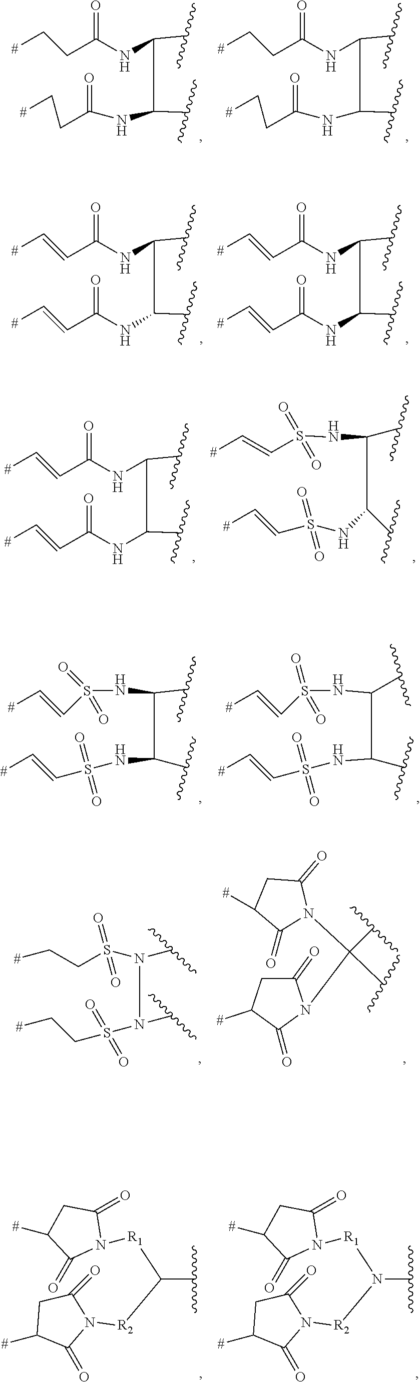

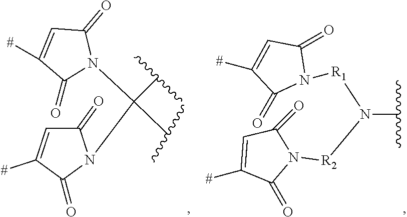

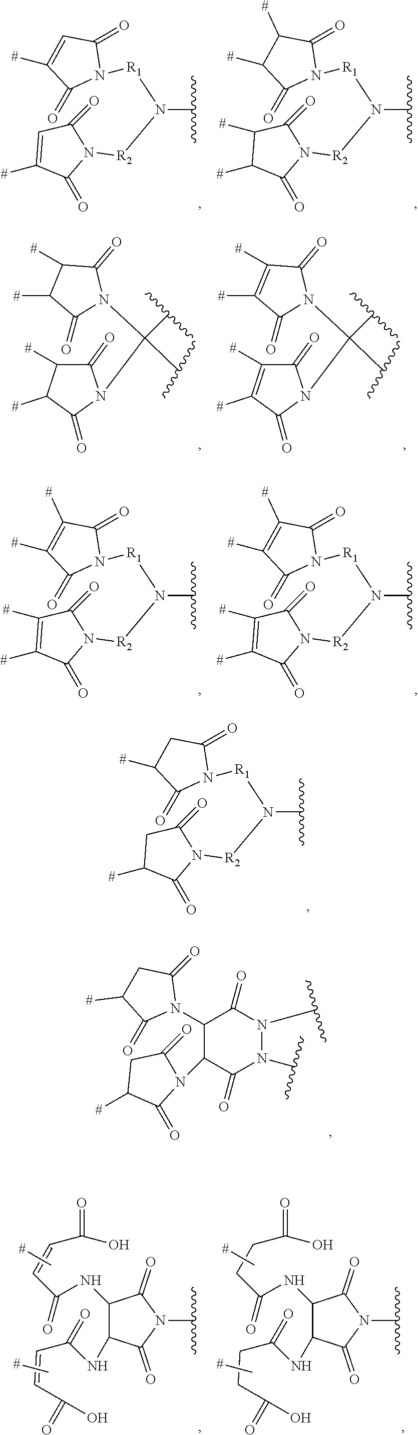

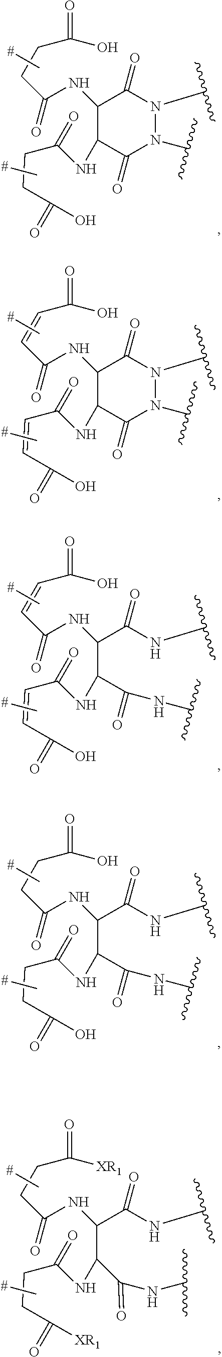

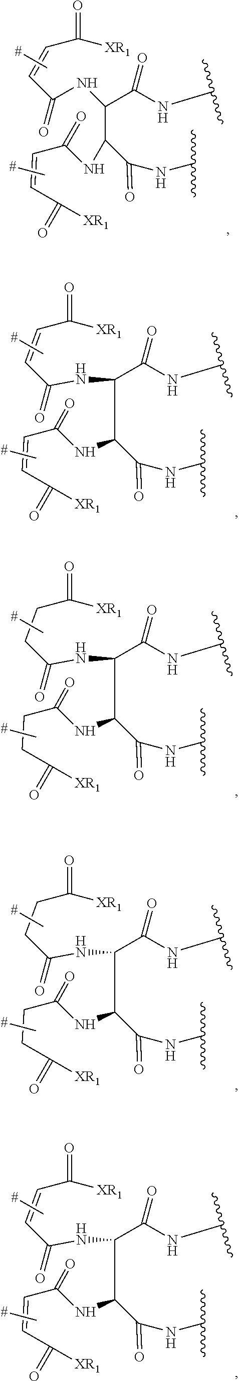

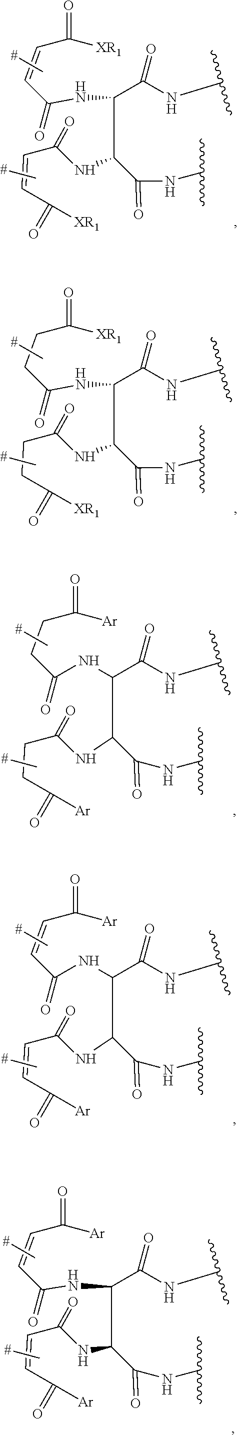

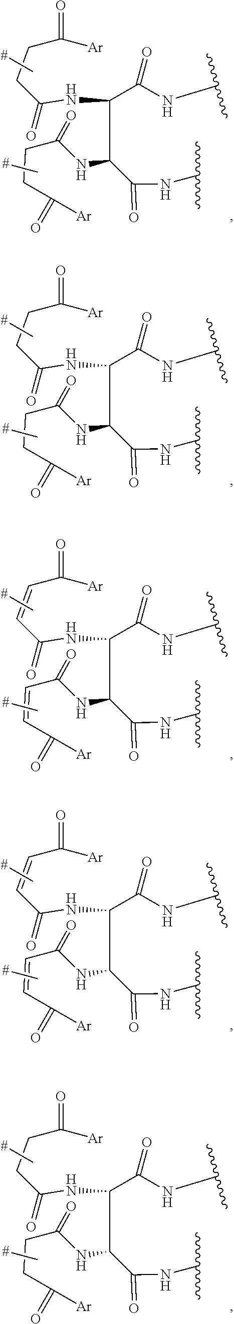

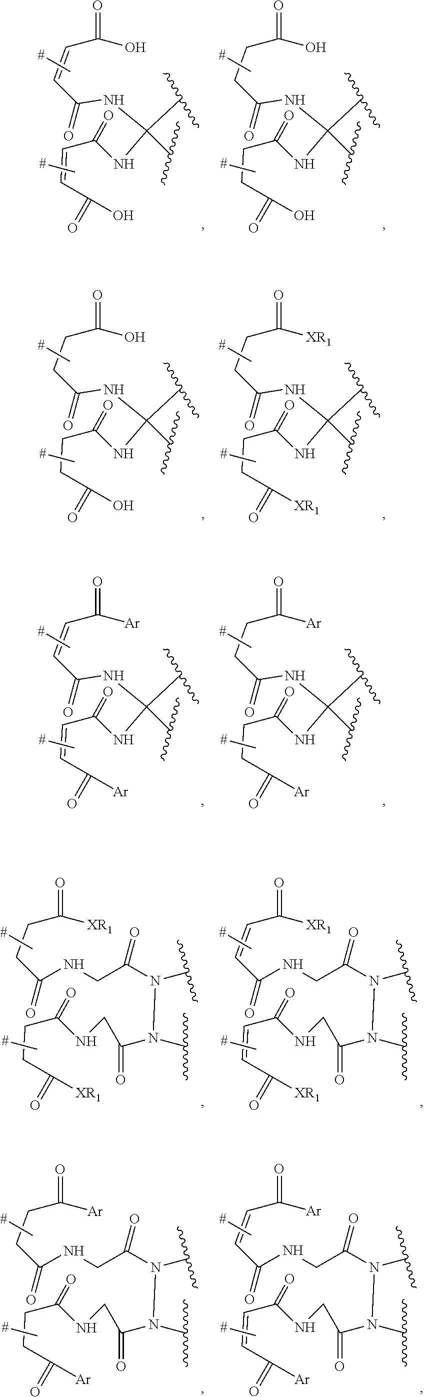

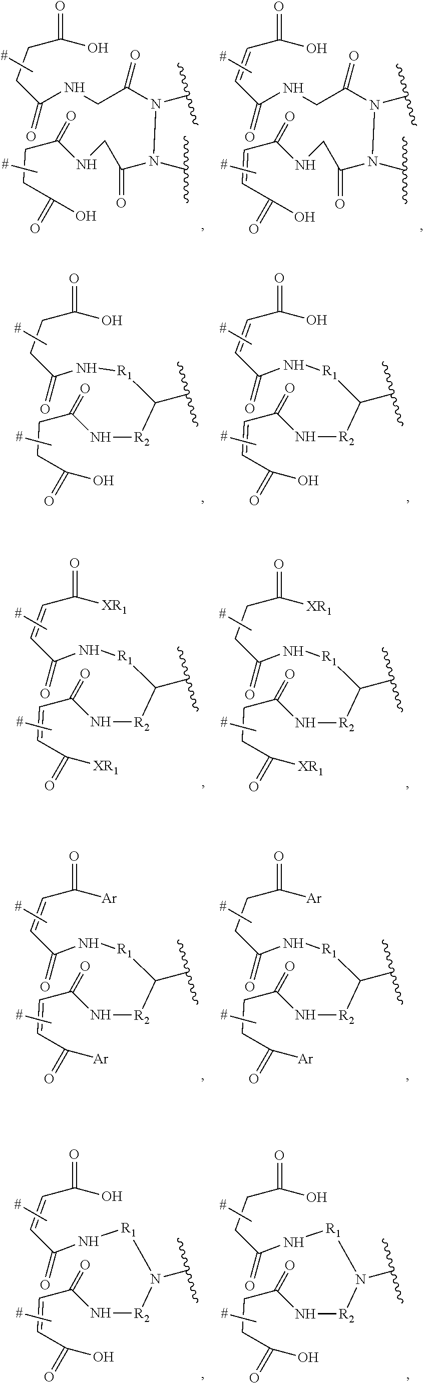

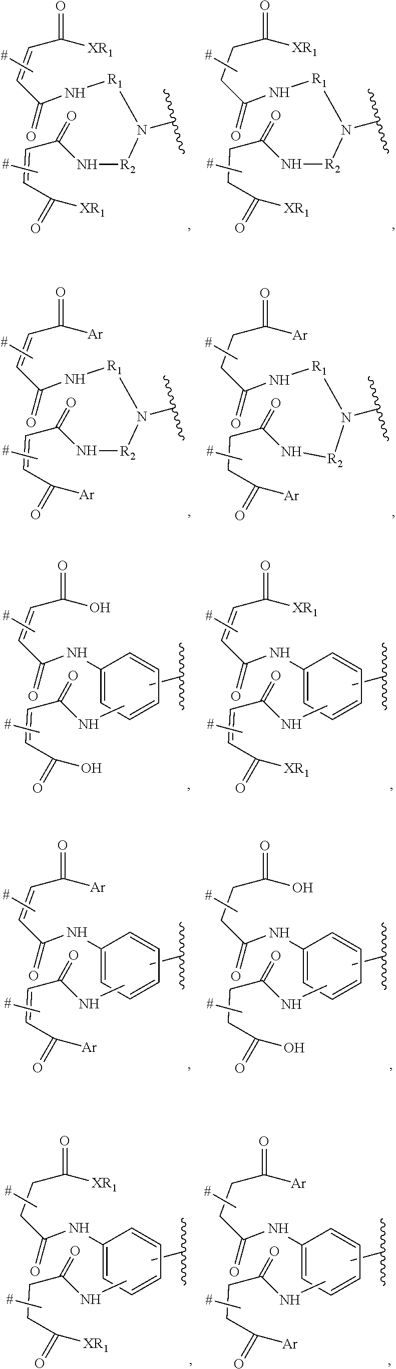

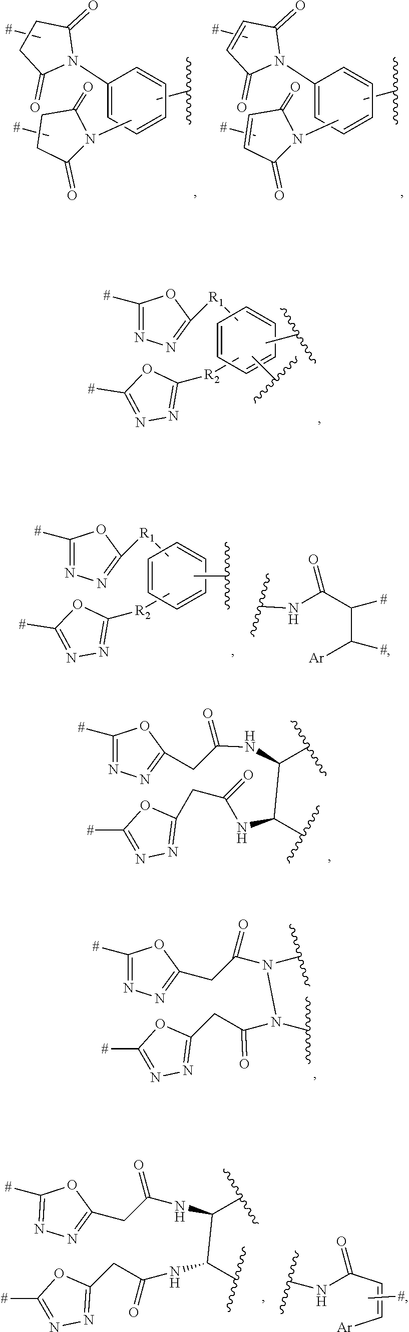

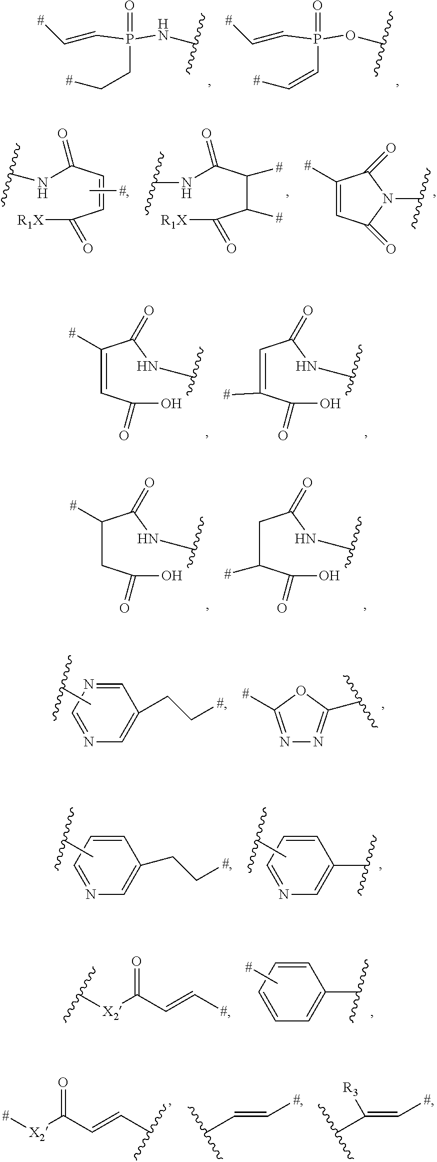

wherein “ [symbol] ” is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; wherein R₁ and R₂ are H, C₁-C₆ of alkyl or a peptide containing 1~4 units of aminoacids; X₁′, X₂′ and X are O, NH, S, CH₂; the conneting bond “—” in the middle of the two atoms means it can link either one of the two atoms, Ar is an aromatic group;
A₁, A₂, A₃, A₄, A₅ and A₆ are independently an affinity ligand including affinity peptide and cell-penetrating peptide (CPP), a ligand for bombesin receptors/neurotensin receptors (including neuropeptide-Y receptors), and/or a nucleoside antimetabolite/analog, which have either the synergy with D₁ or D₂, or enhanced affinity for mAb.

In some embodiments, the affinity ligand/peptide including the cell-penetrating peptide (CPP) to a receptor on a faulty cell is EC50<100 nM. The CPP is a linear or cyclo peptide having less than 50 amino acids and containing one, two, or several arginines or lysines and enables to internalize (trafficking) over 40% of the ligand bound on a cell or help to internalize 40% of ADCs bound on a cell to cross the cell membrane in 2 hours.

In some embodiments, the affinity ligand/peptide including the cell-penetrating peptide (CPP) is independently selected from:

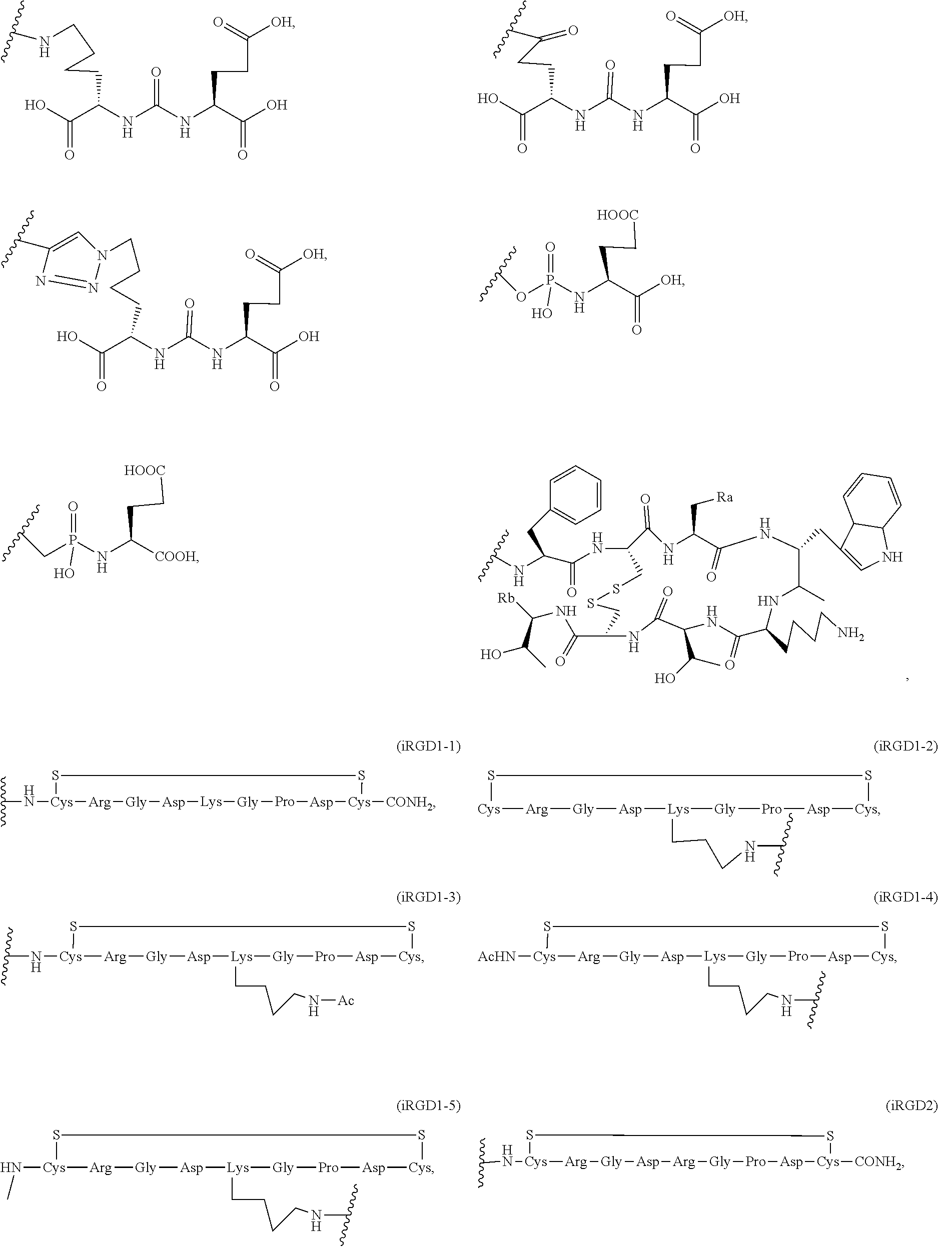

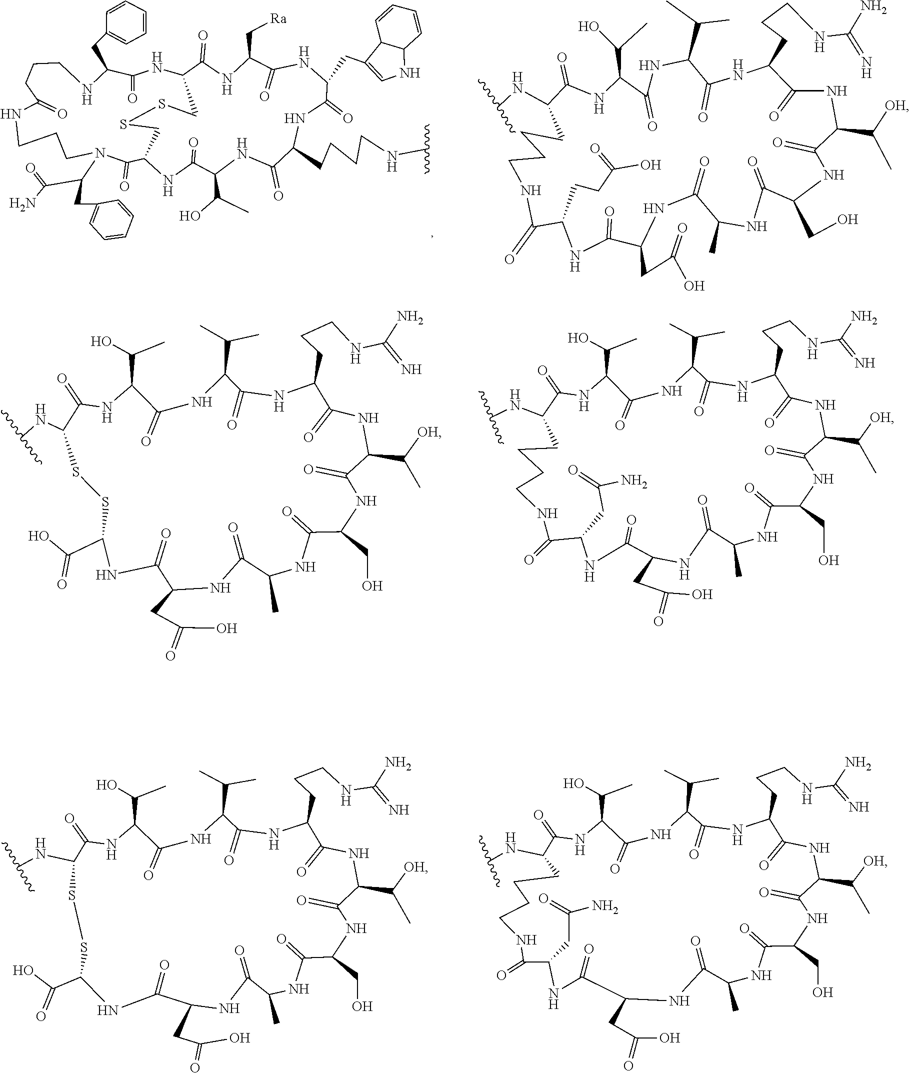

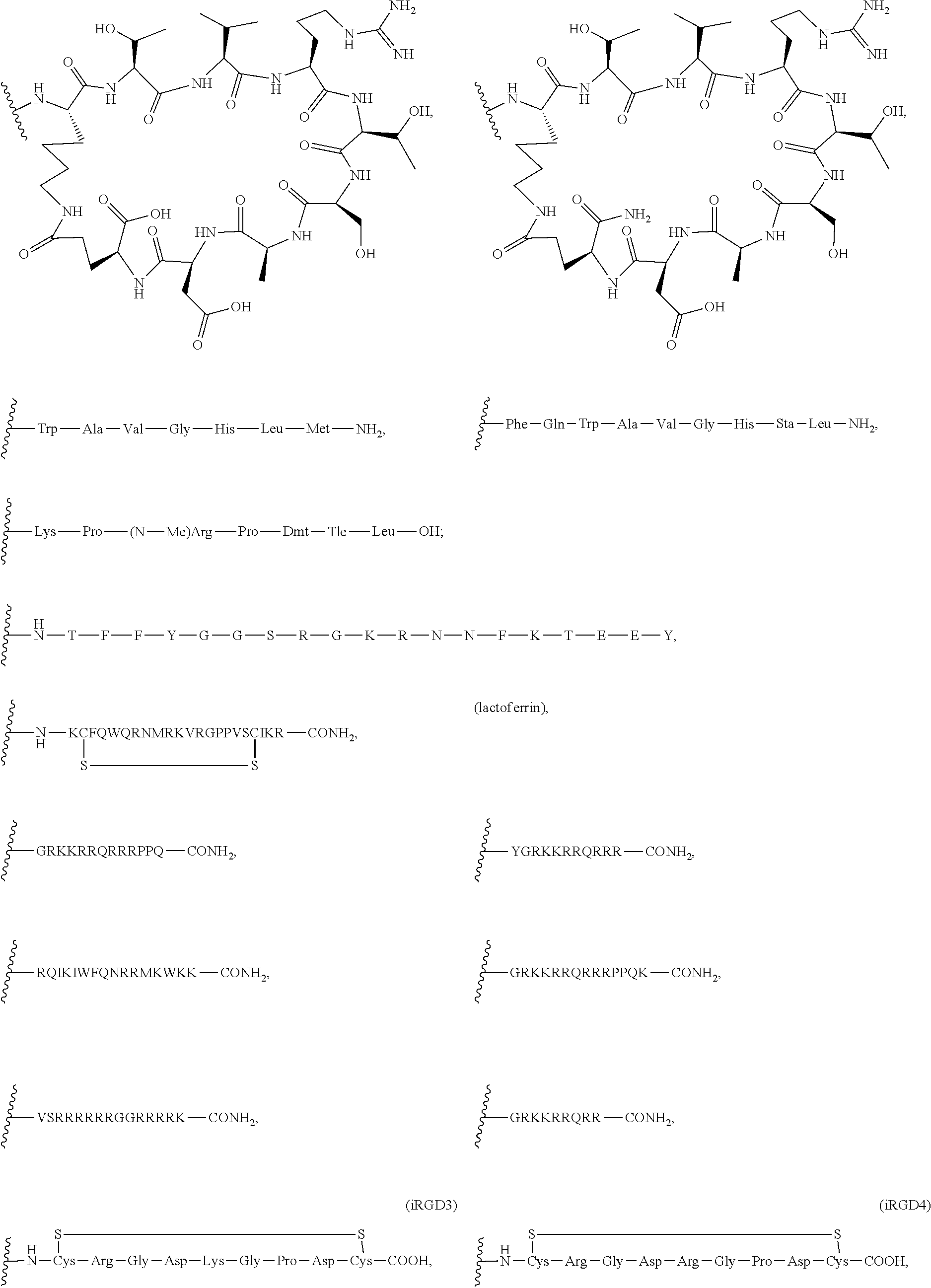

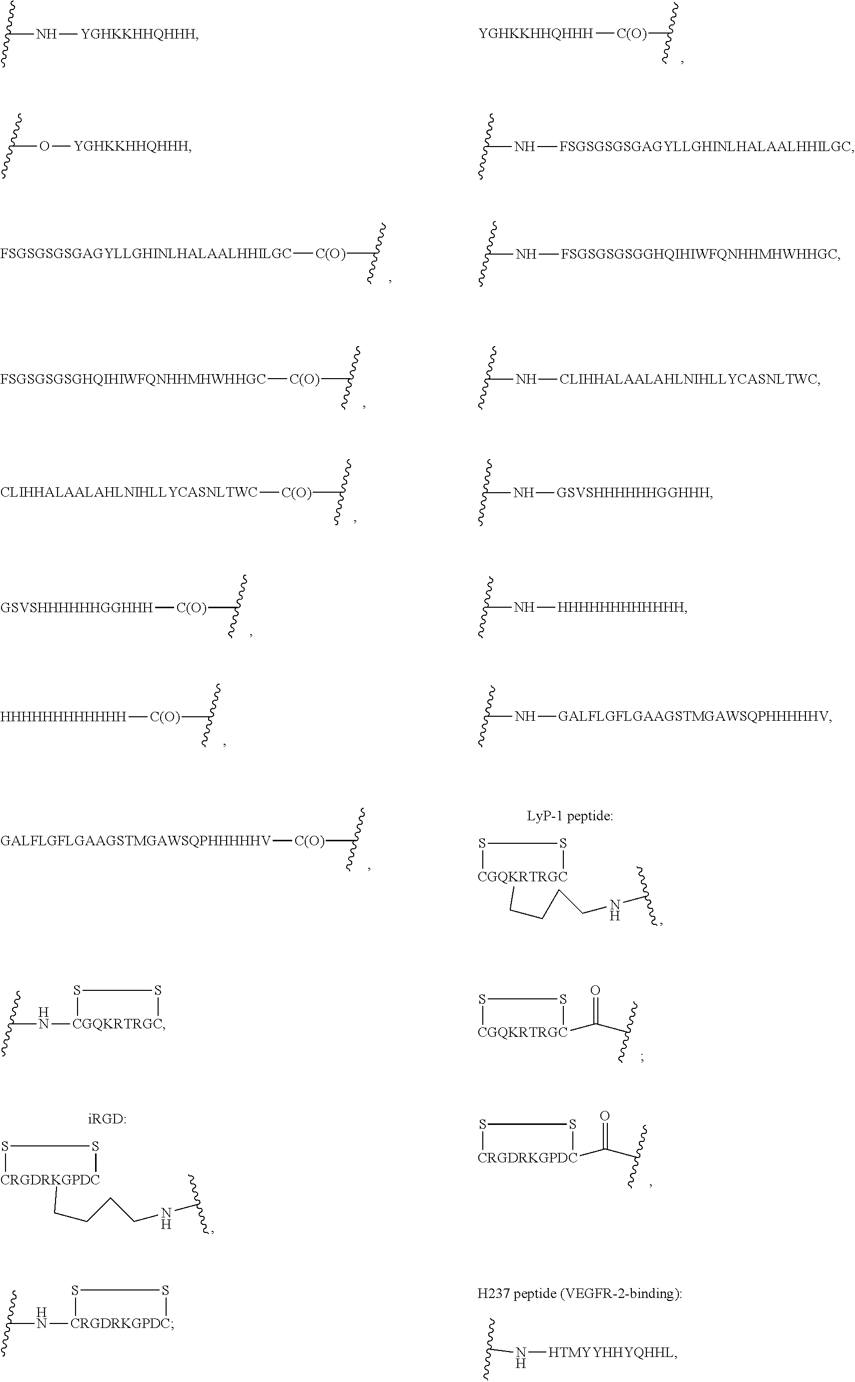

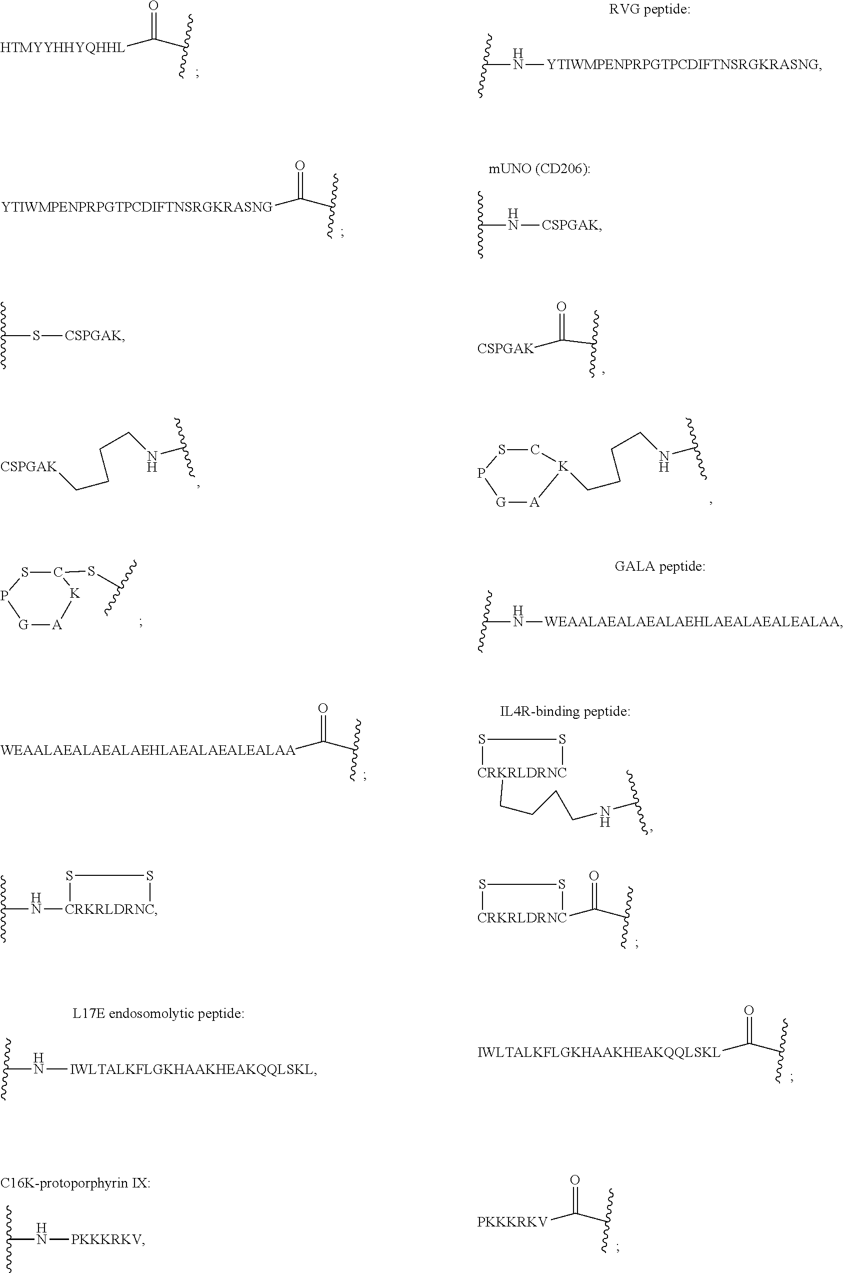

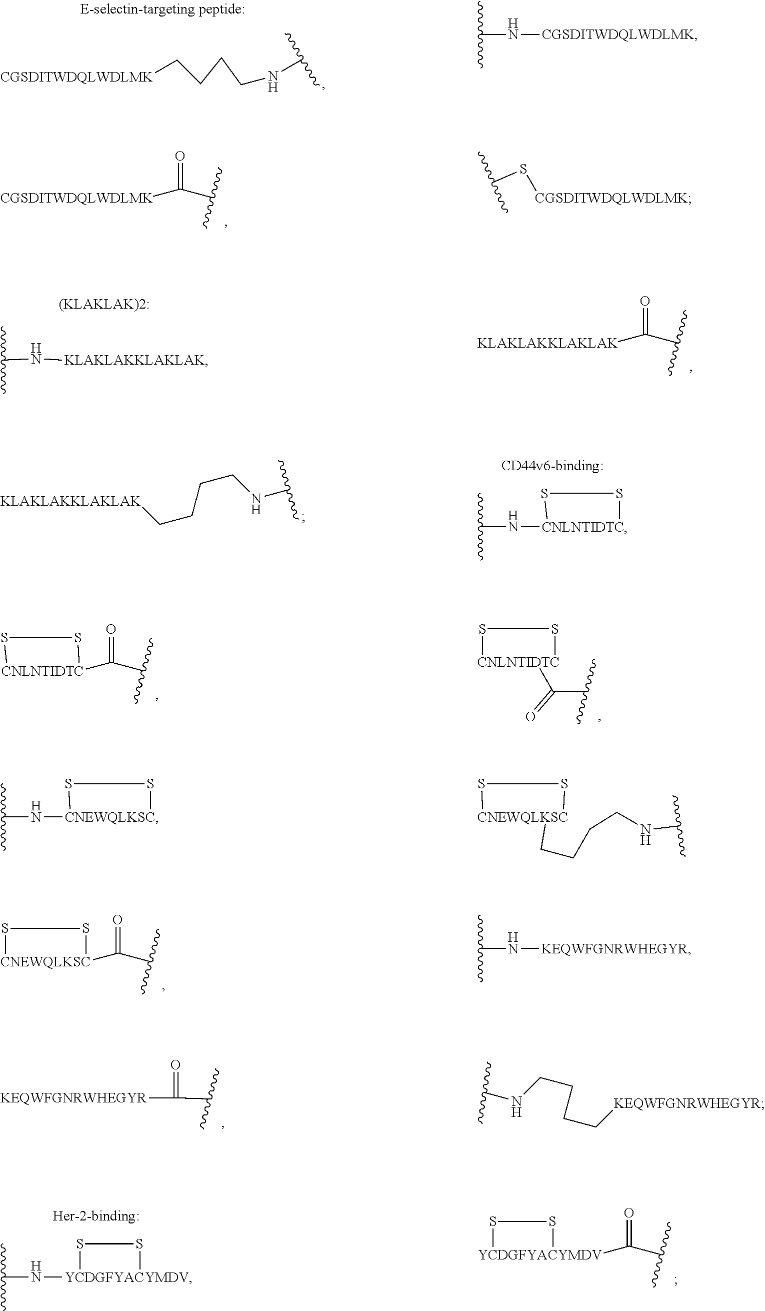

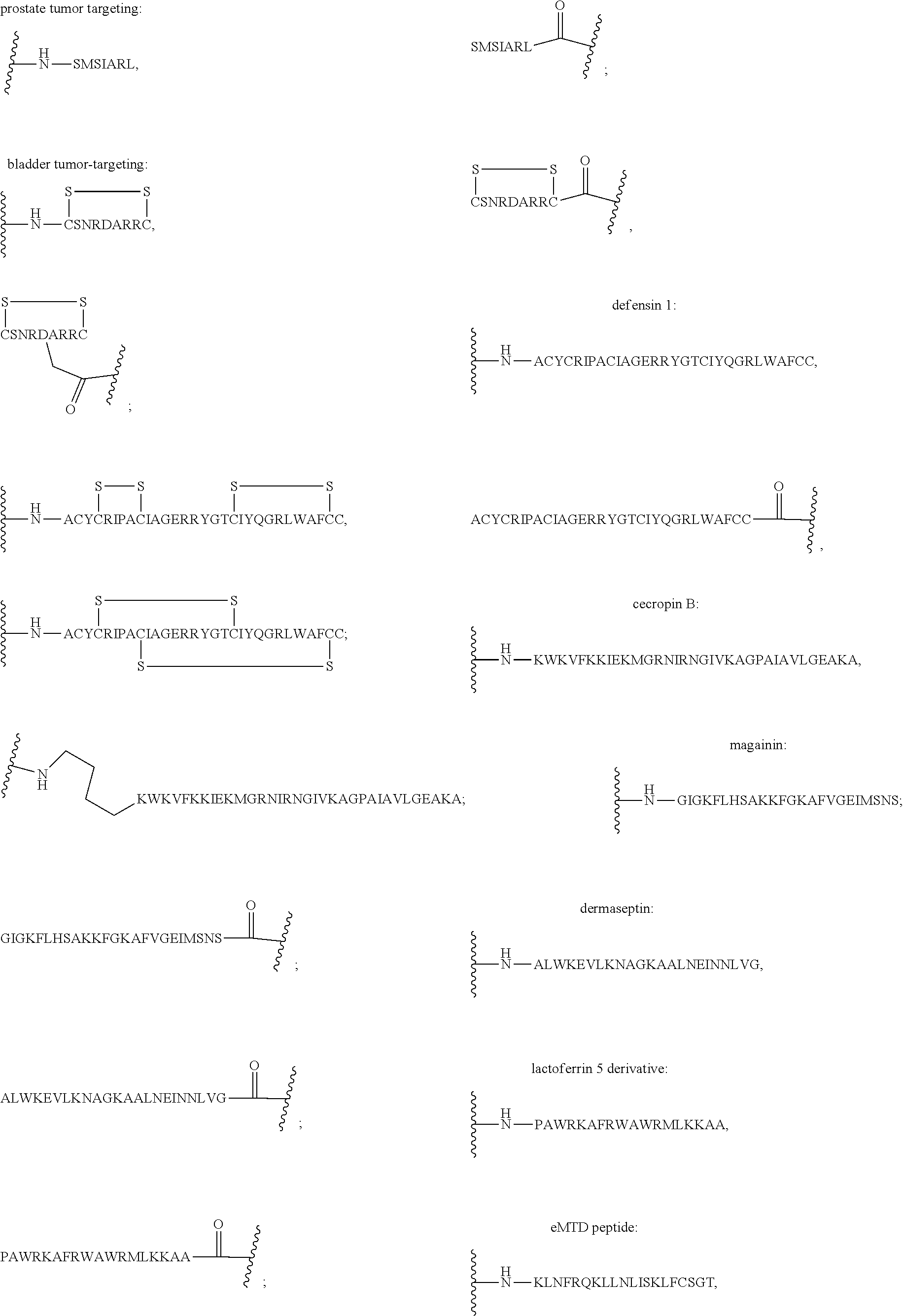

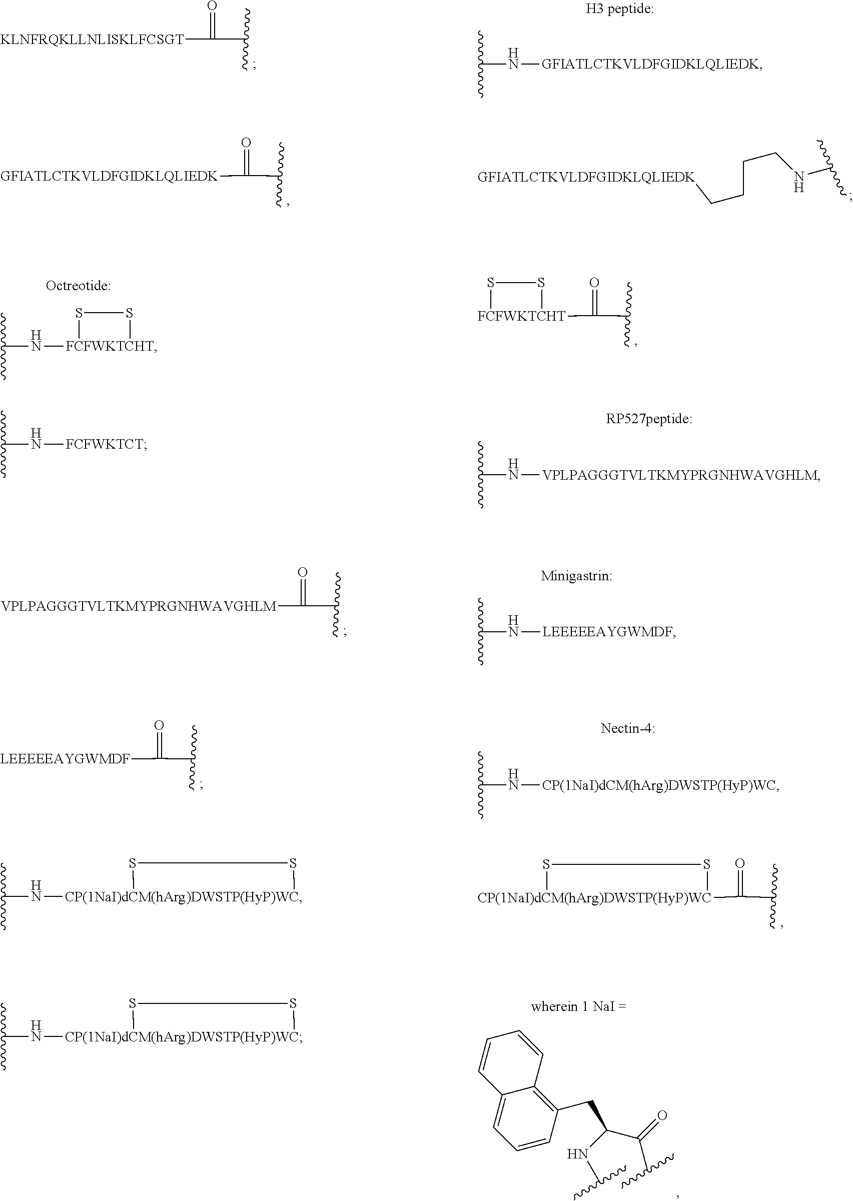

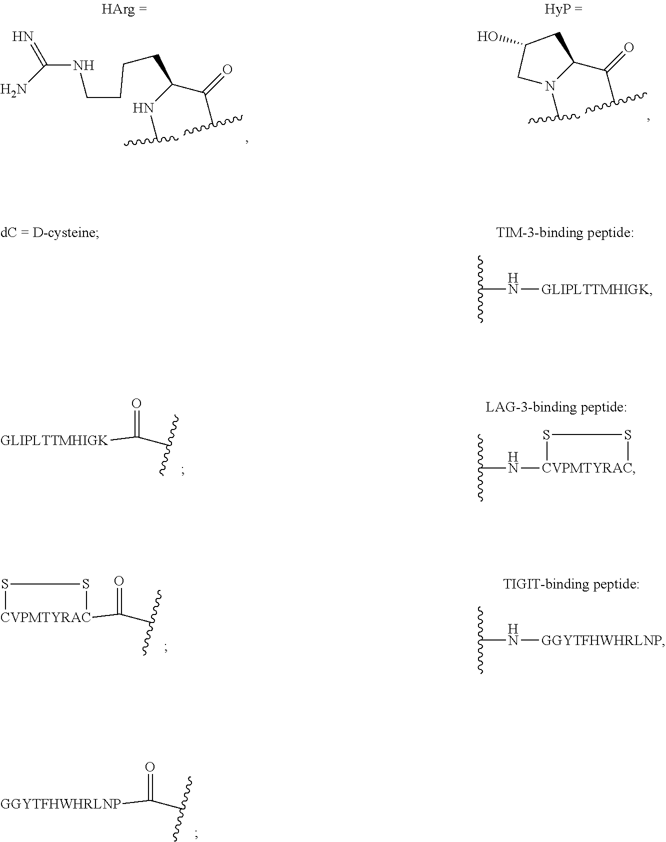

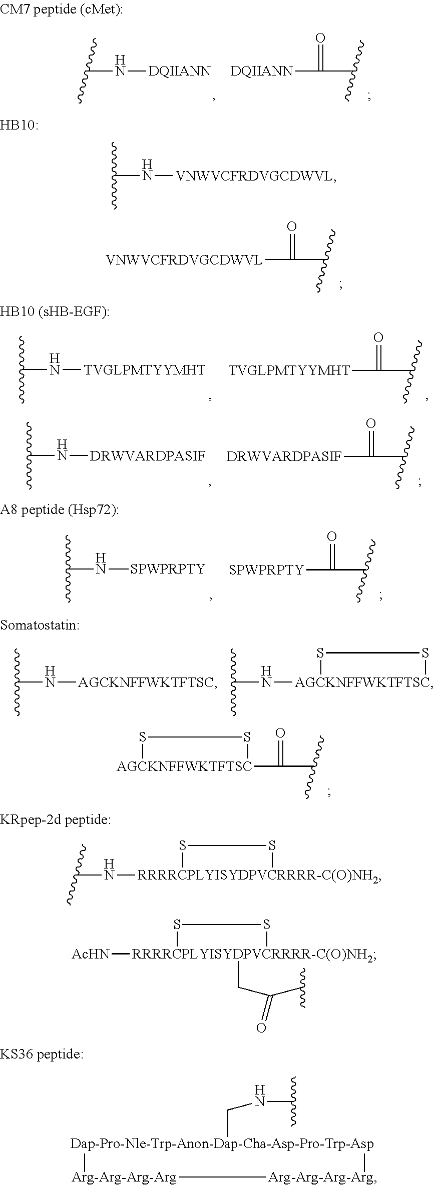

wherein Dap is (S)-2,3-Diaminopropanoic acid, Ne is L-Norleucine, Anon is (S)-2-Aminononanoic acid, Cha is L-Cyclohexylalanine. The others are natural amino acids.

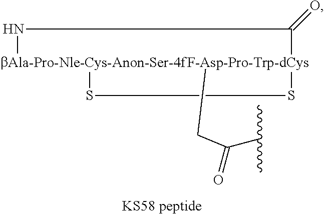

wherein βAla is Beta-alanine, Ne is L-Norleucine, Anon is (S)-2-Aminononanoic acid, 4fF is 4-Fluoro-1-phenylalanine, dCys is D-cysteine;

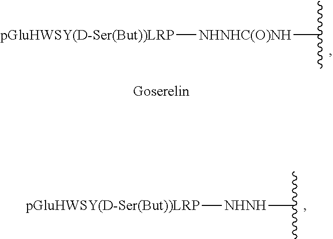

and its analogs as the structures shown below:

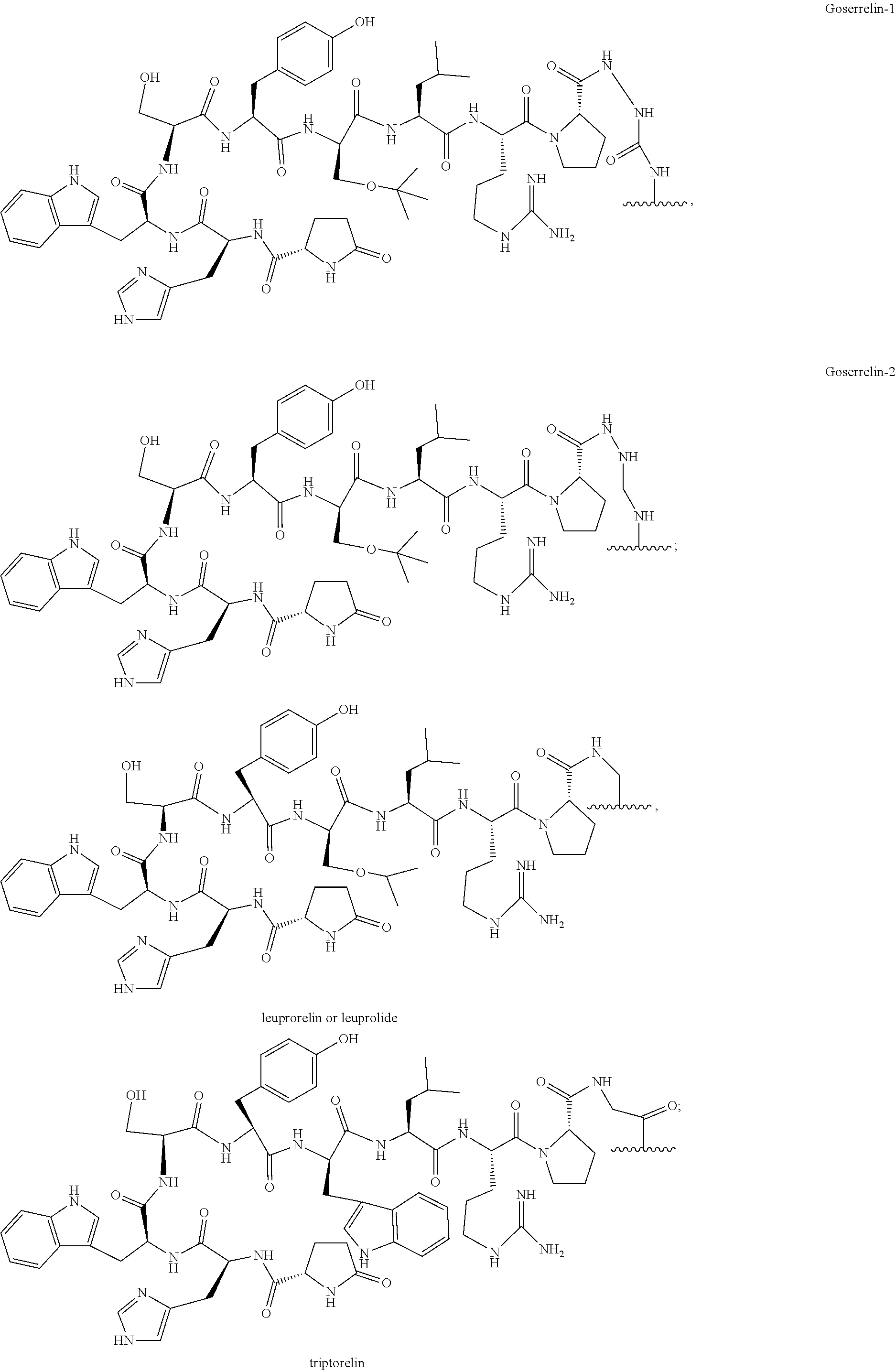

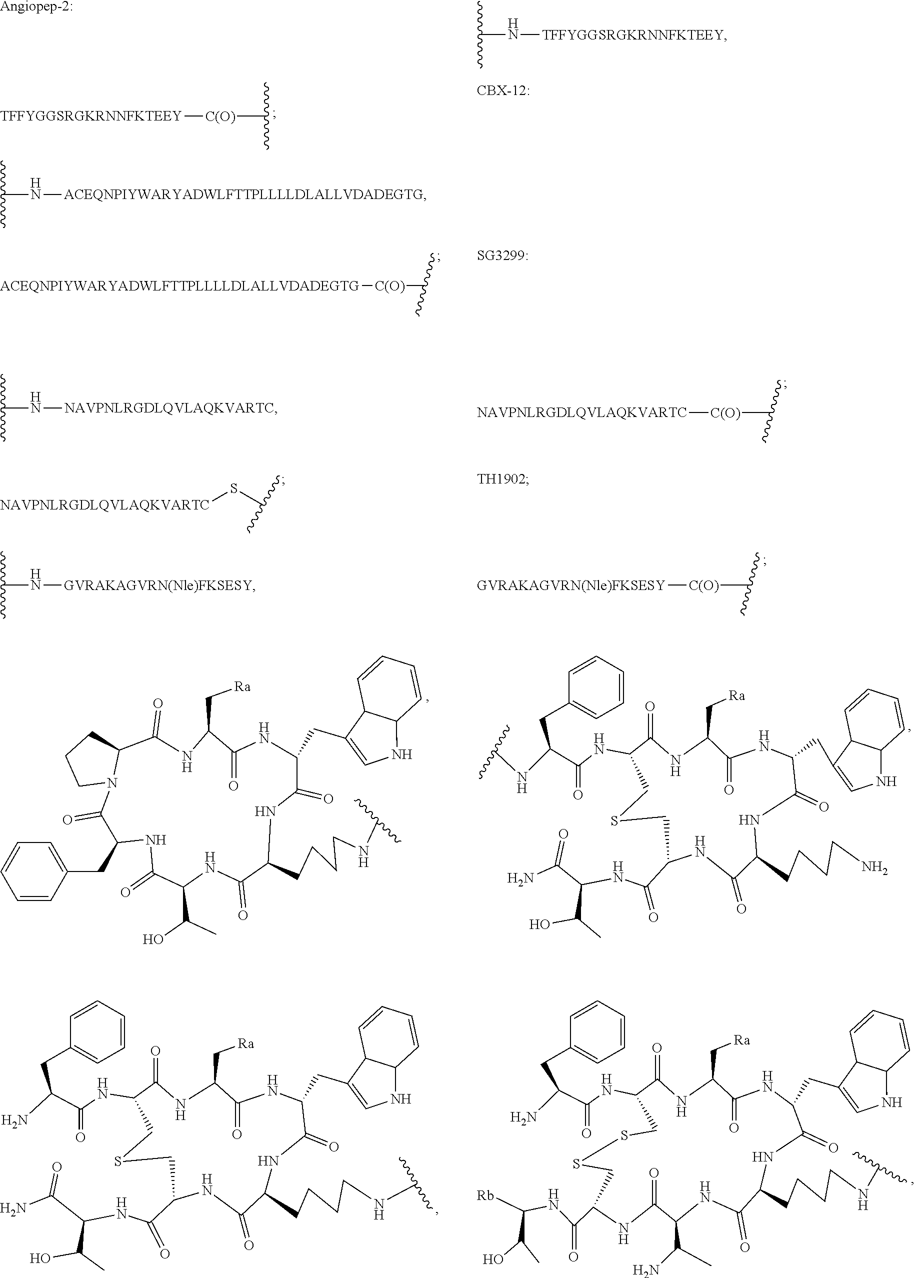

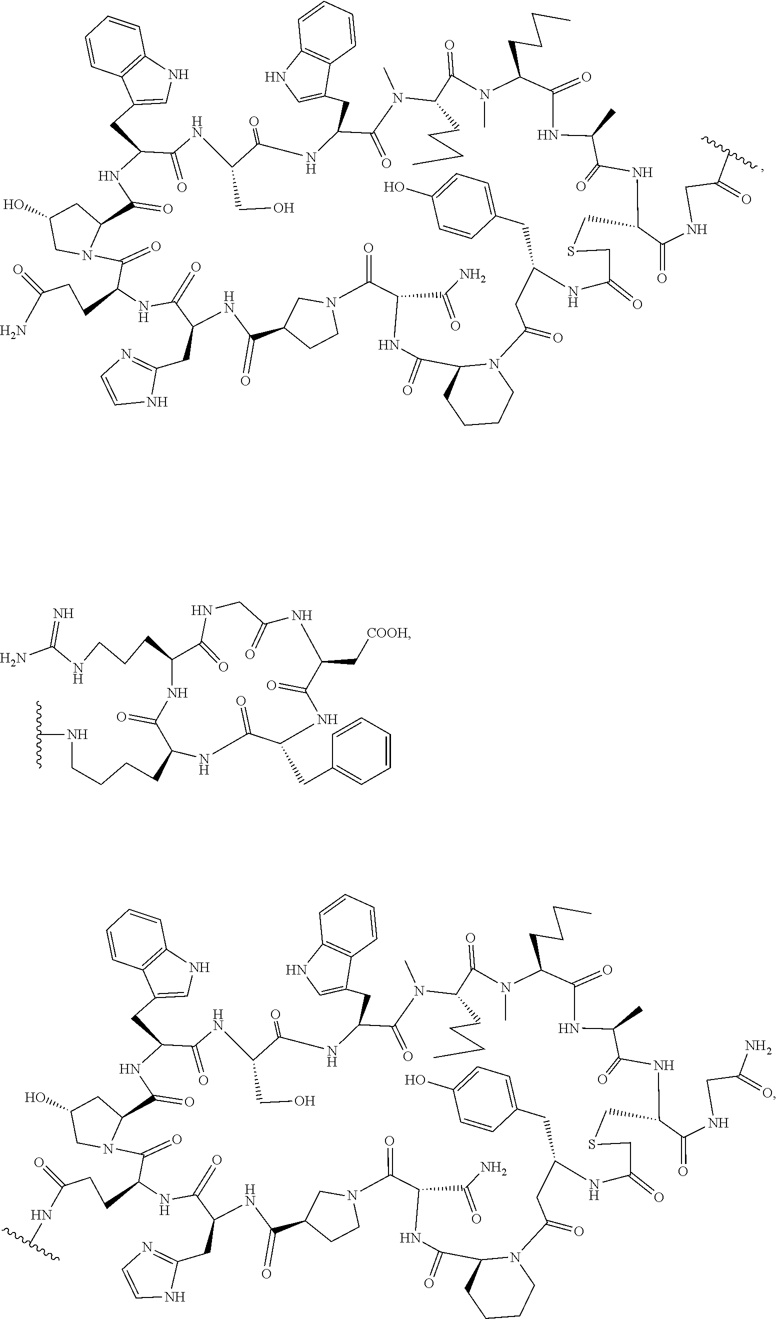

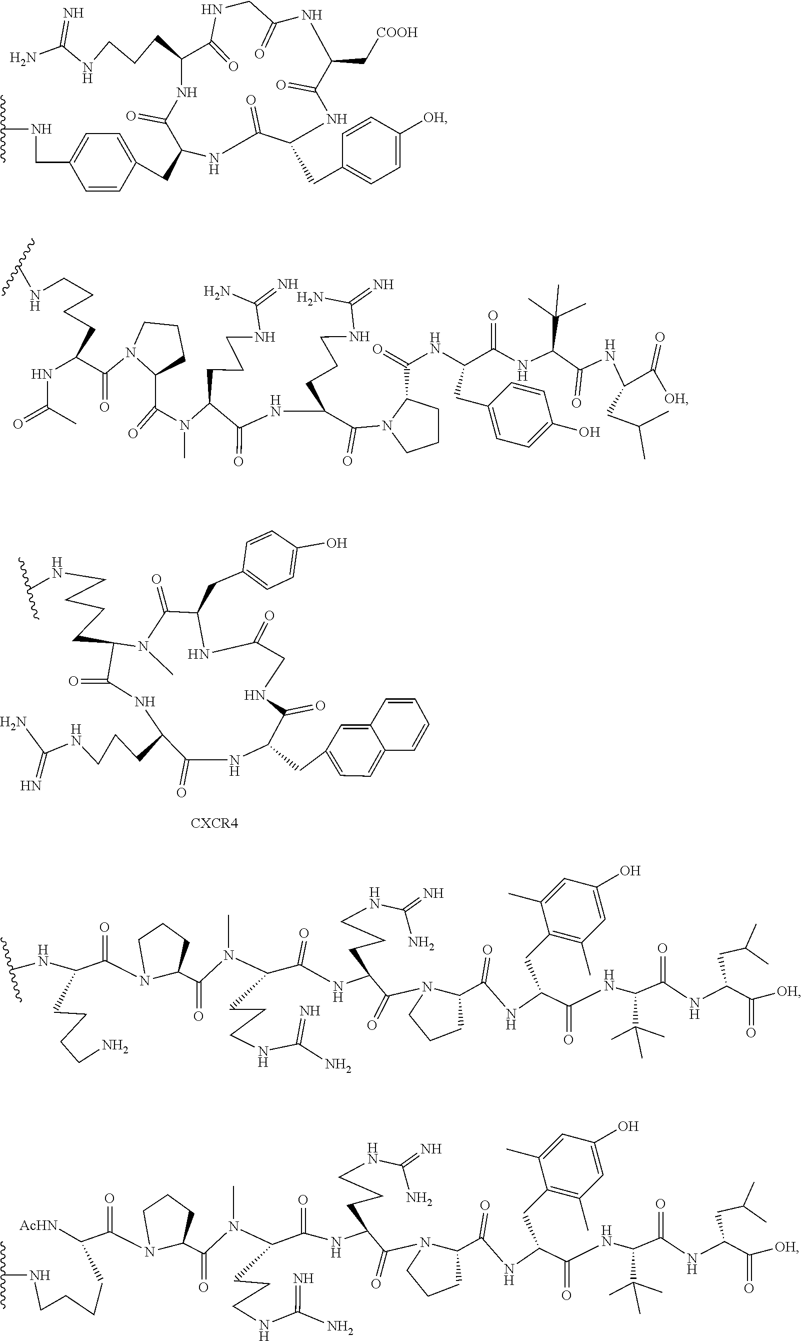

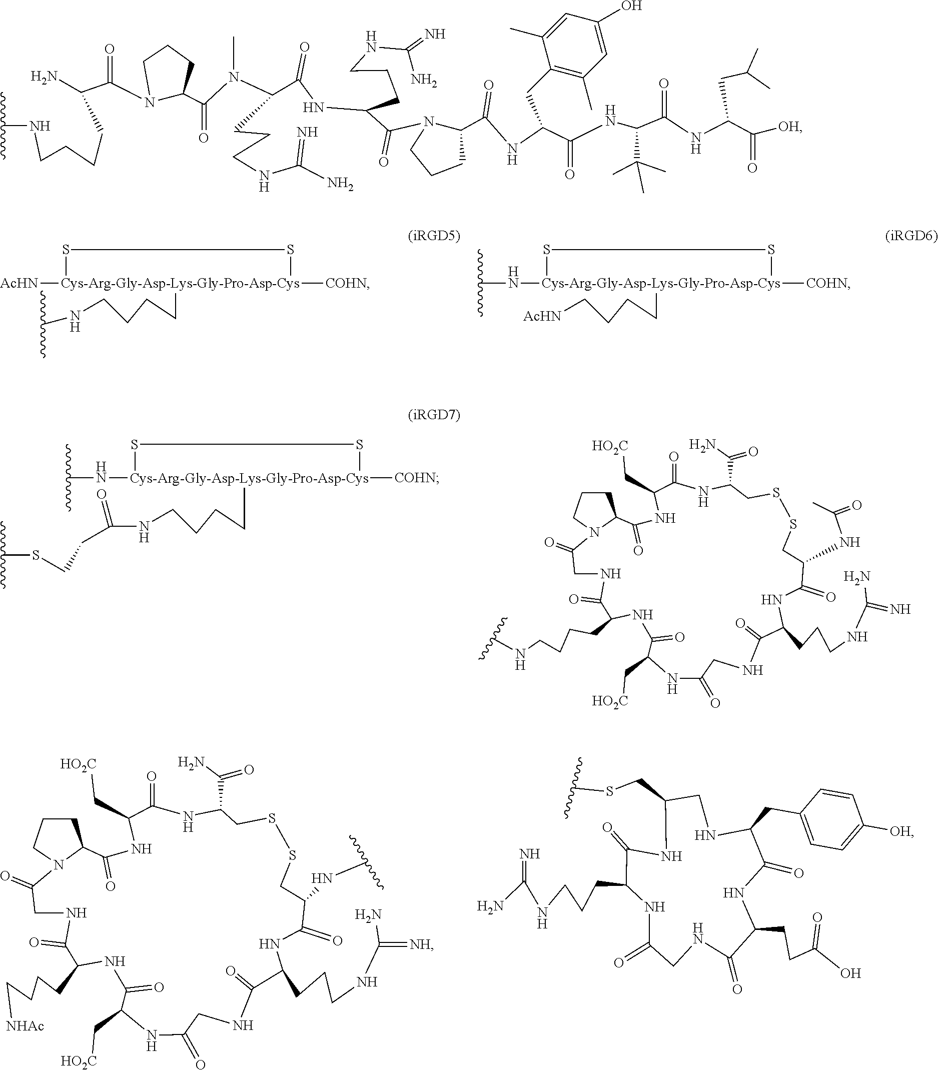

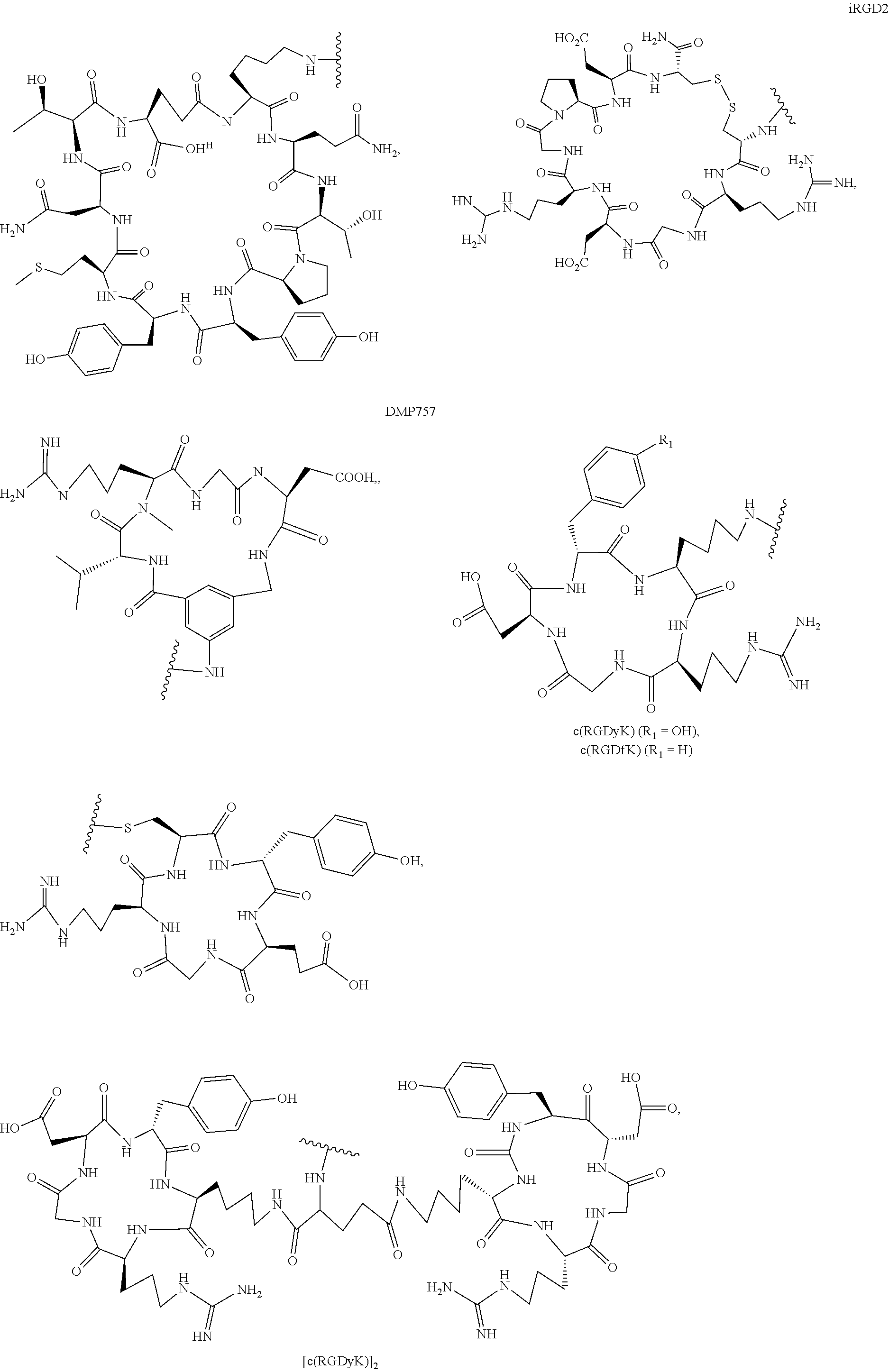

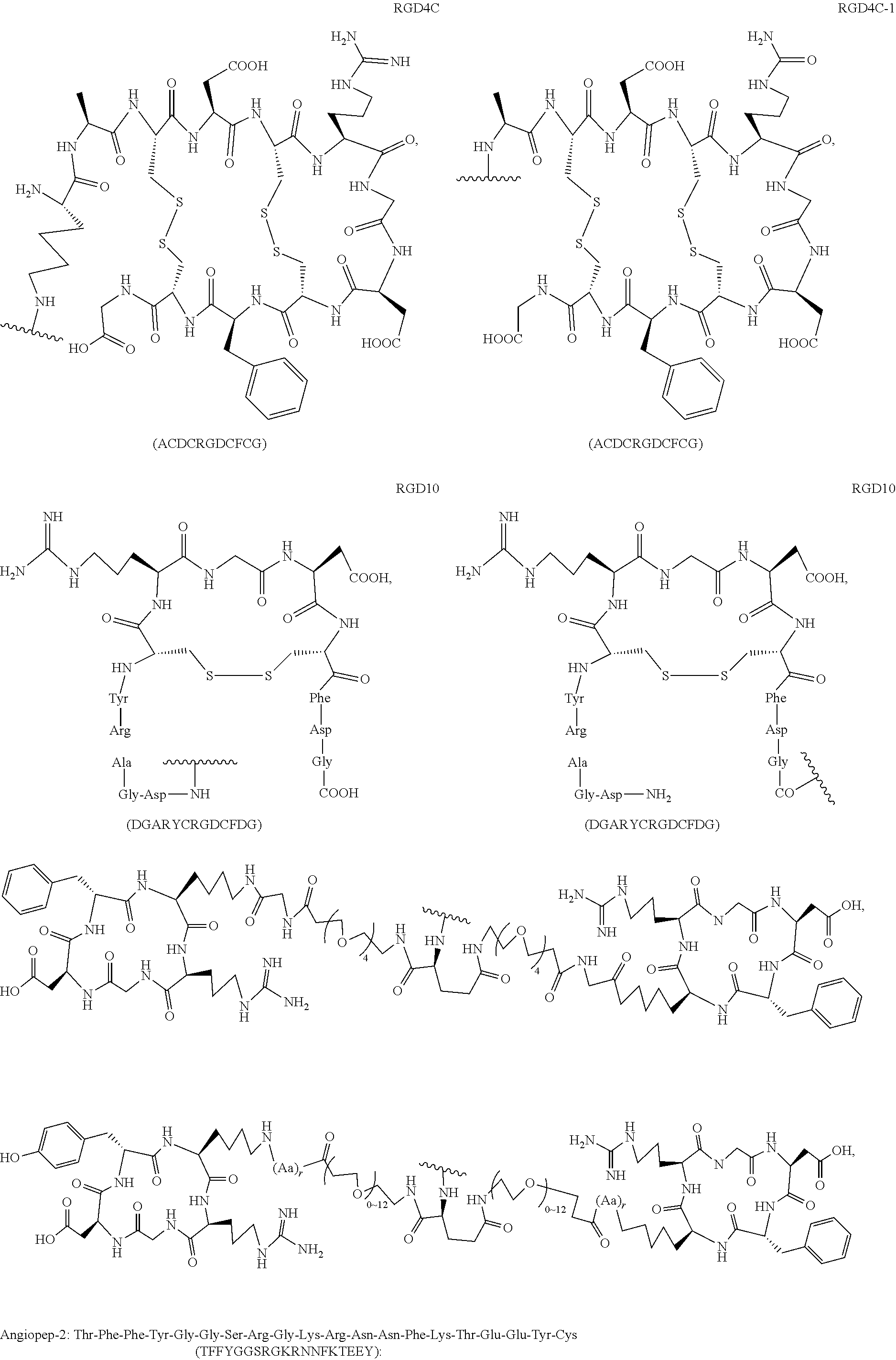

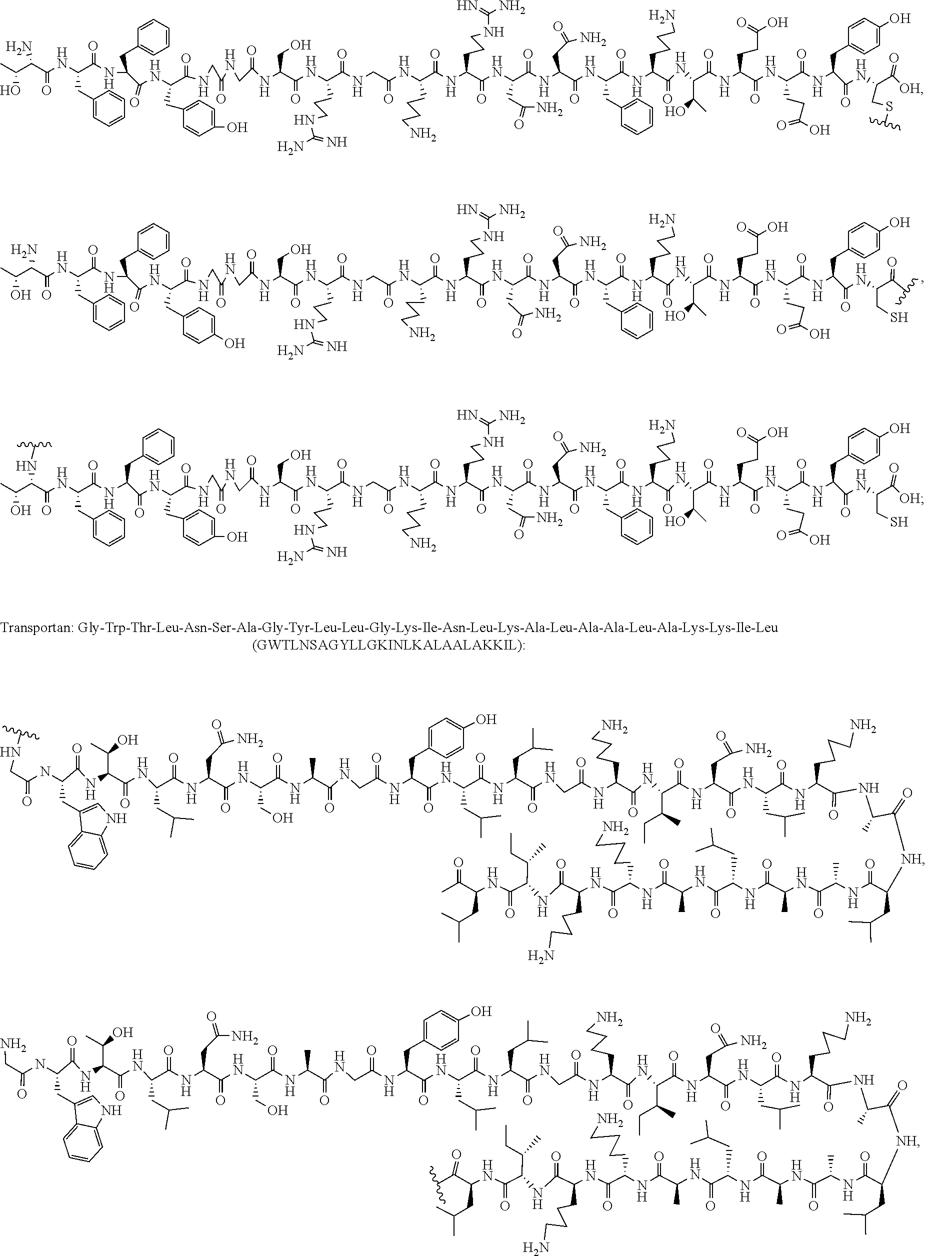

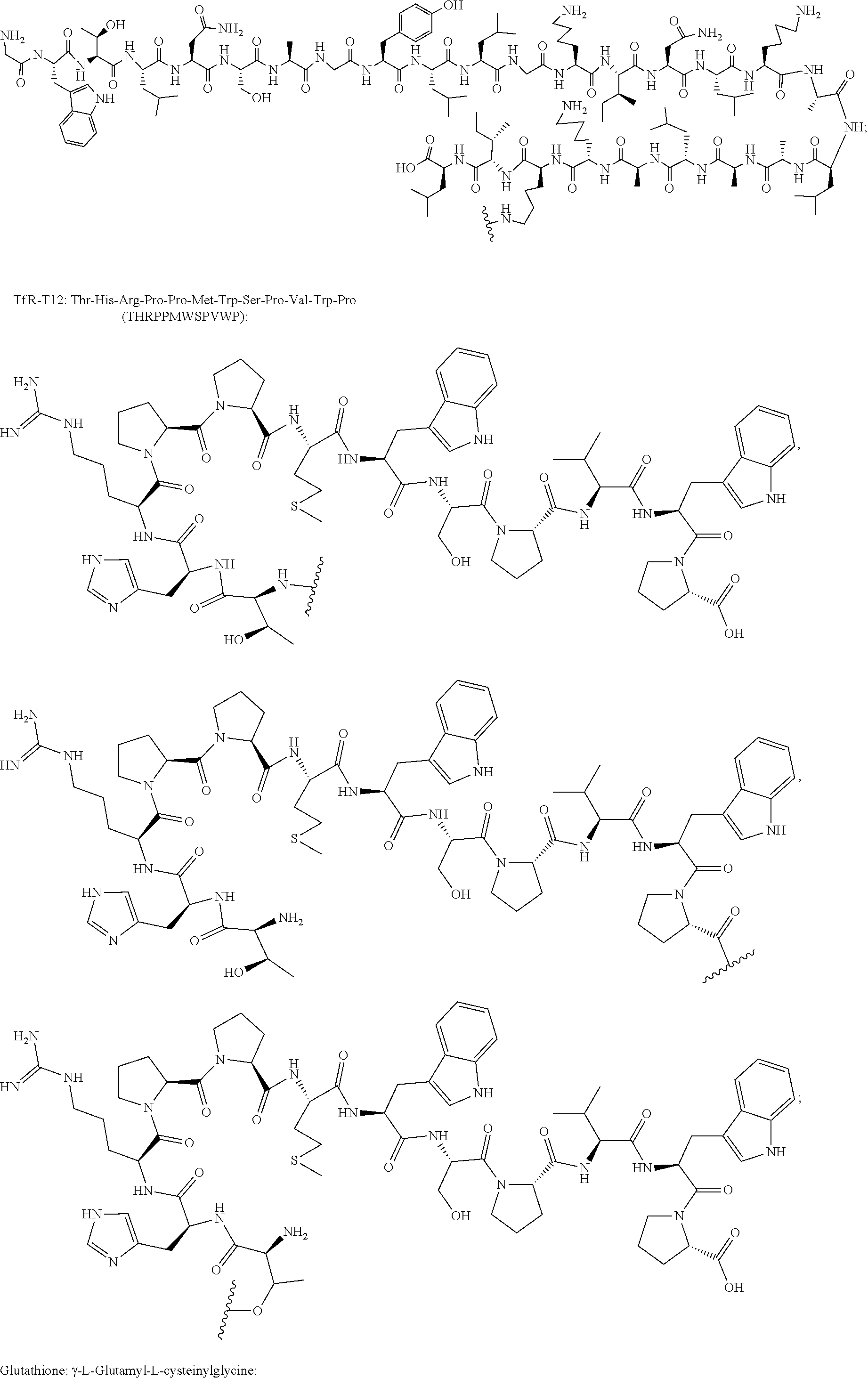

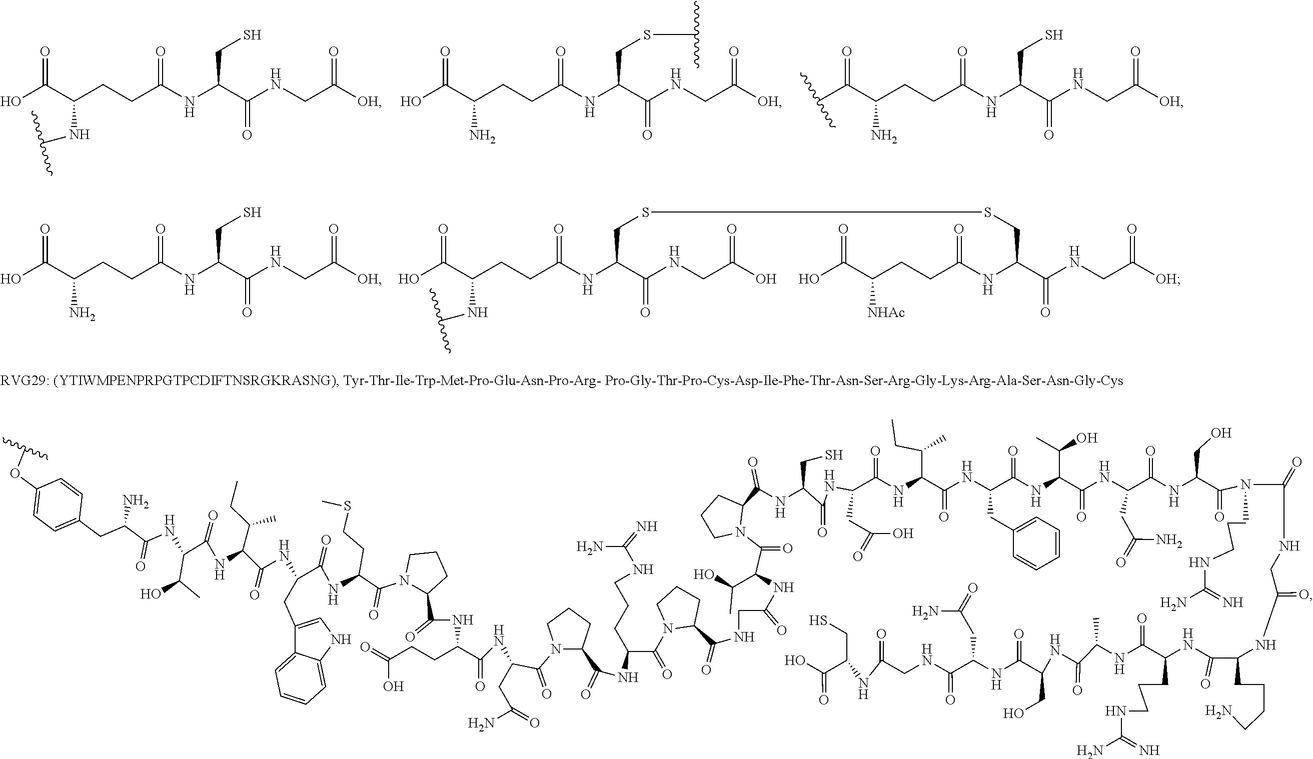

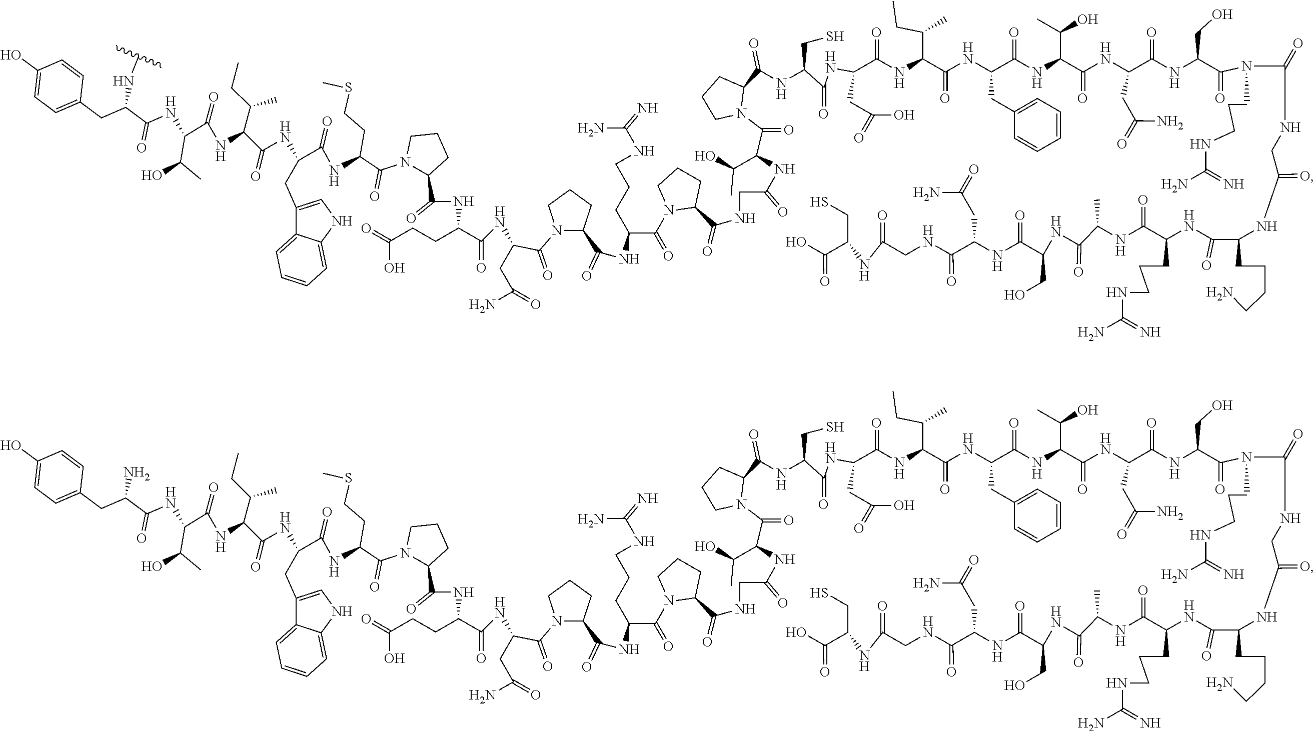

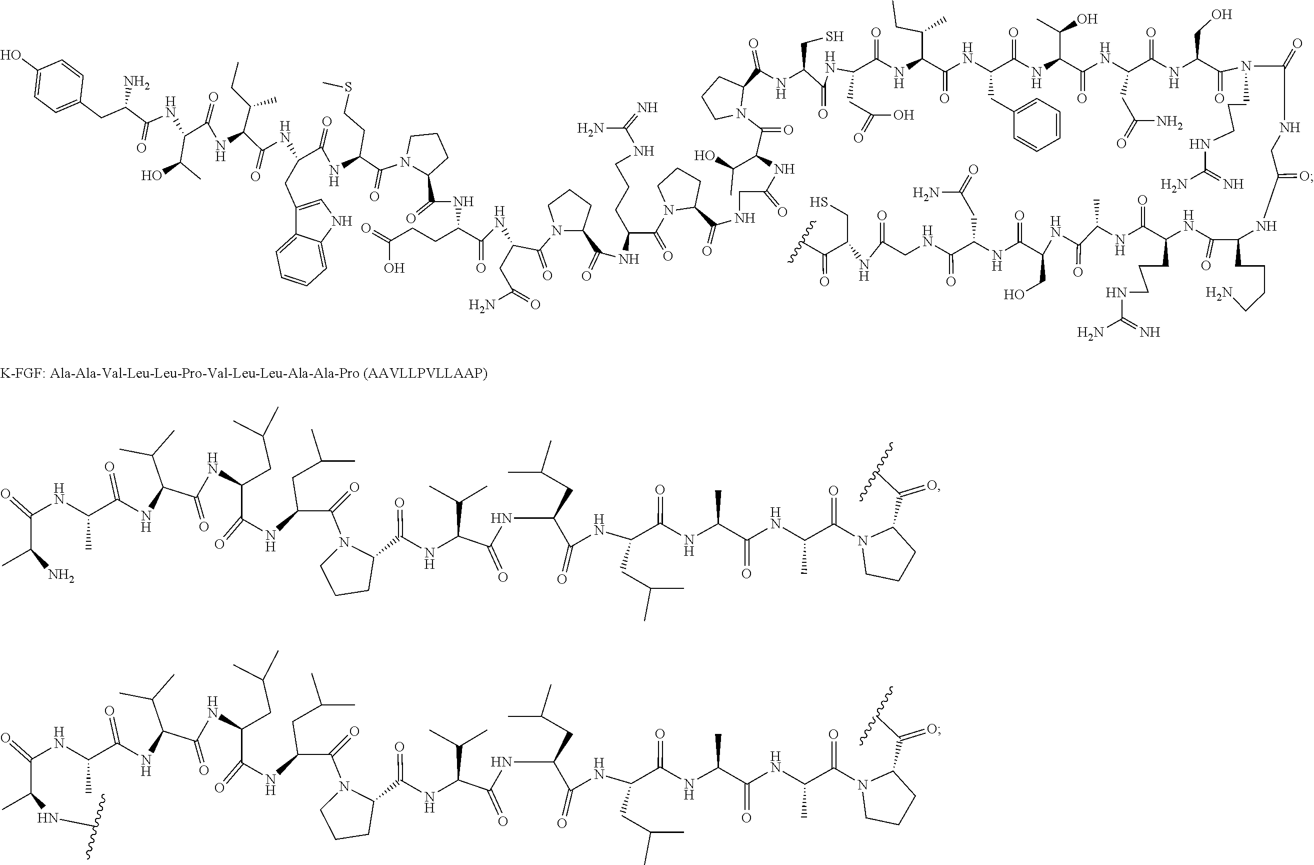

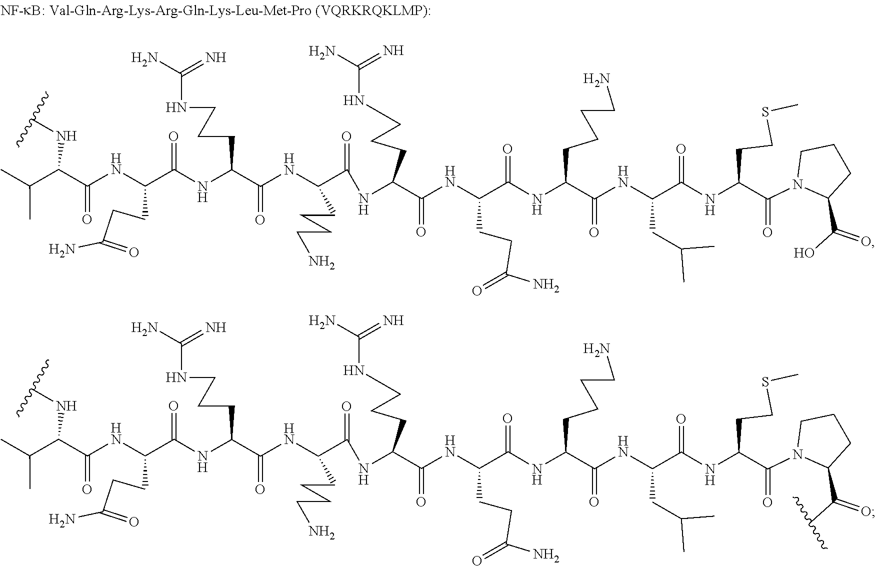

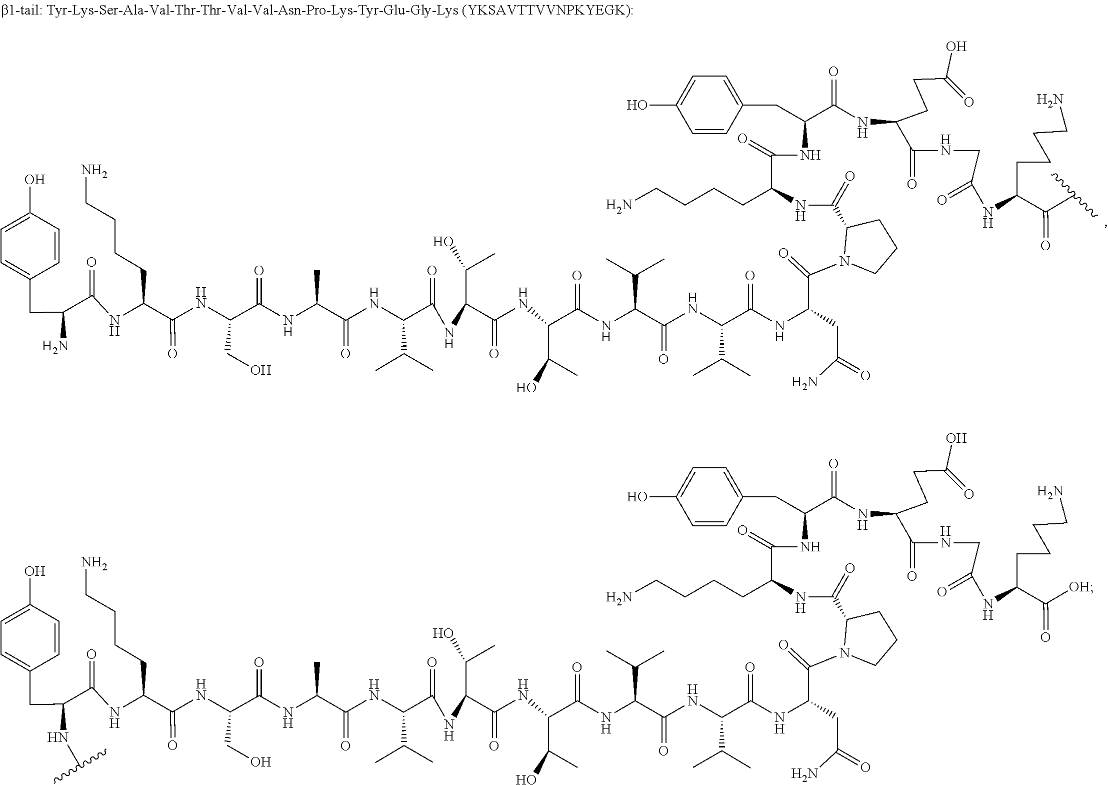

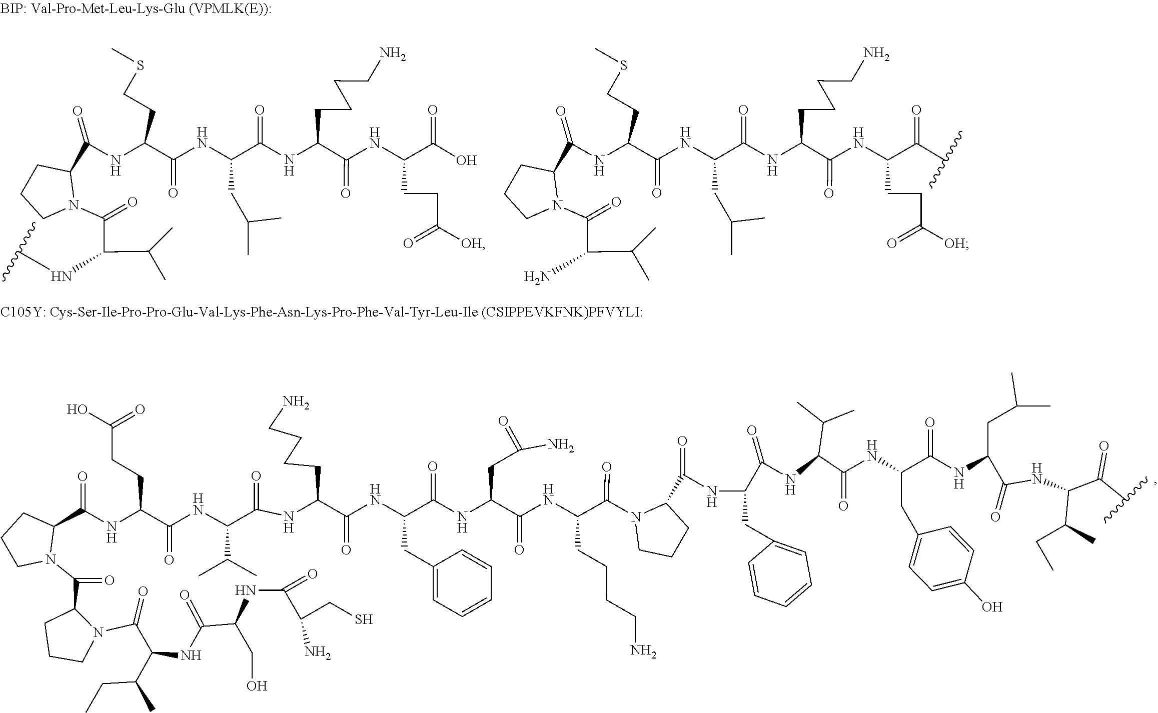

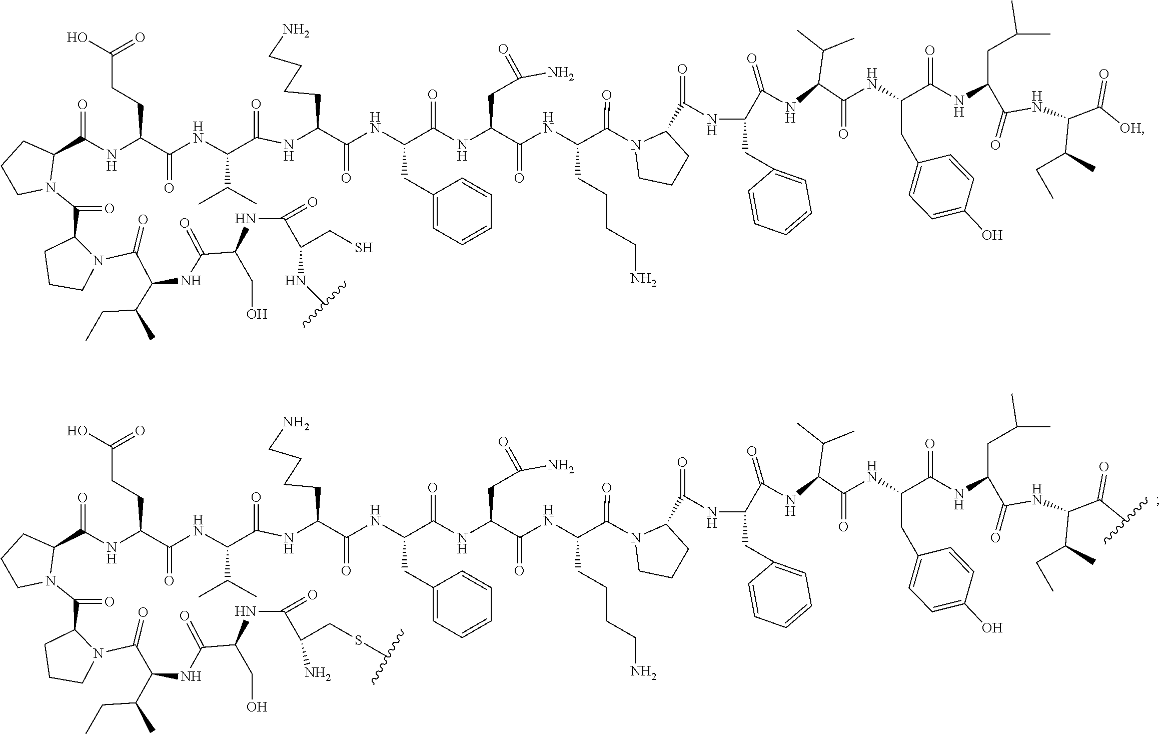

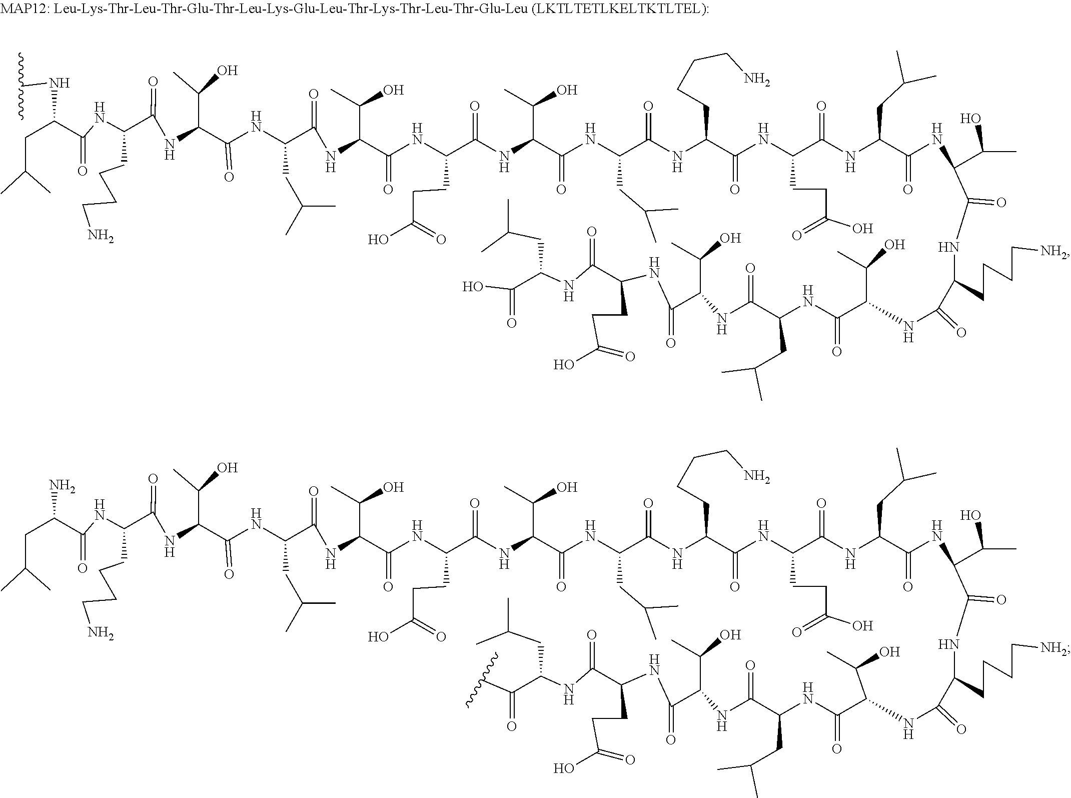

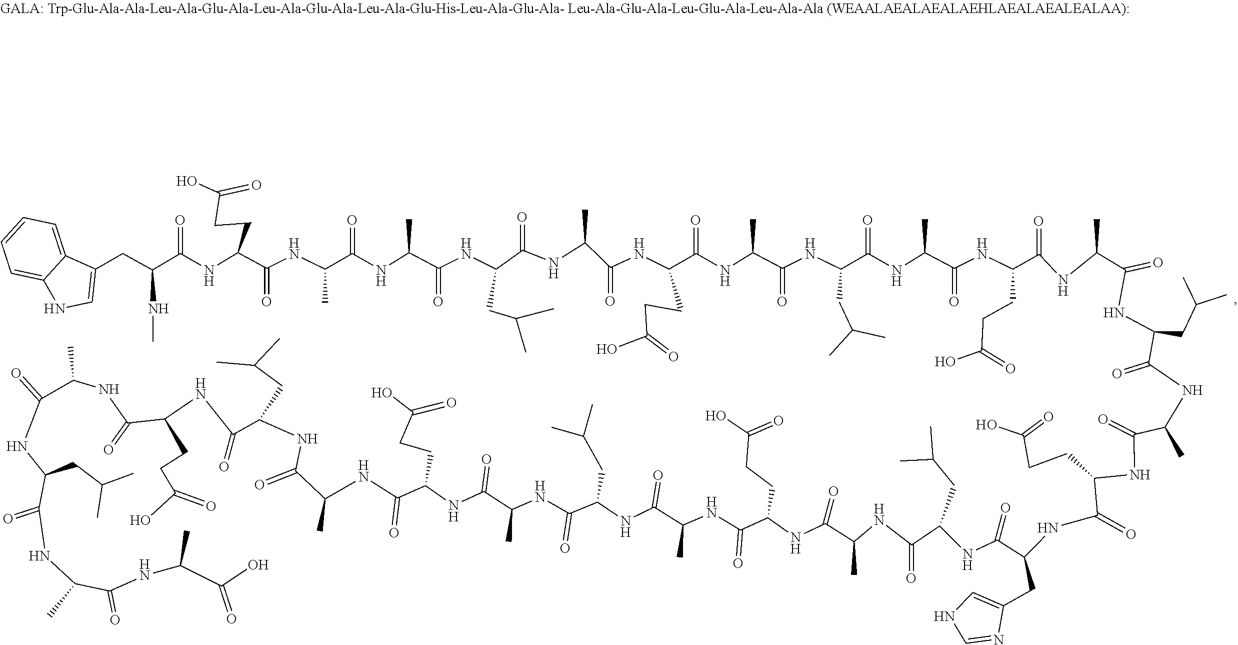

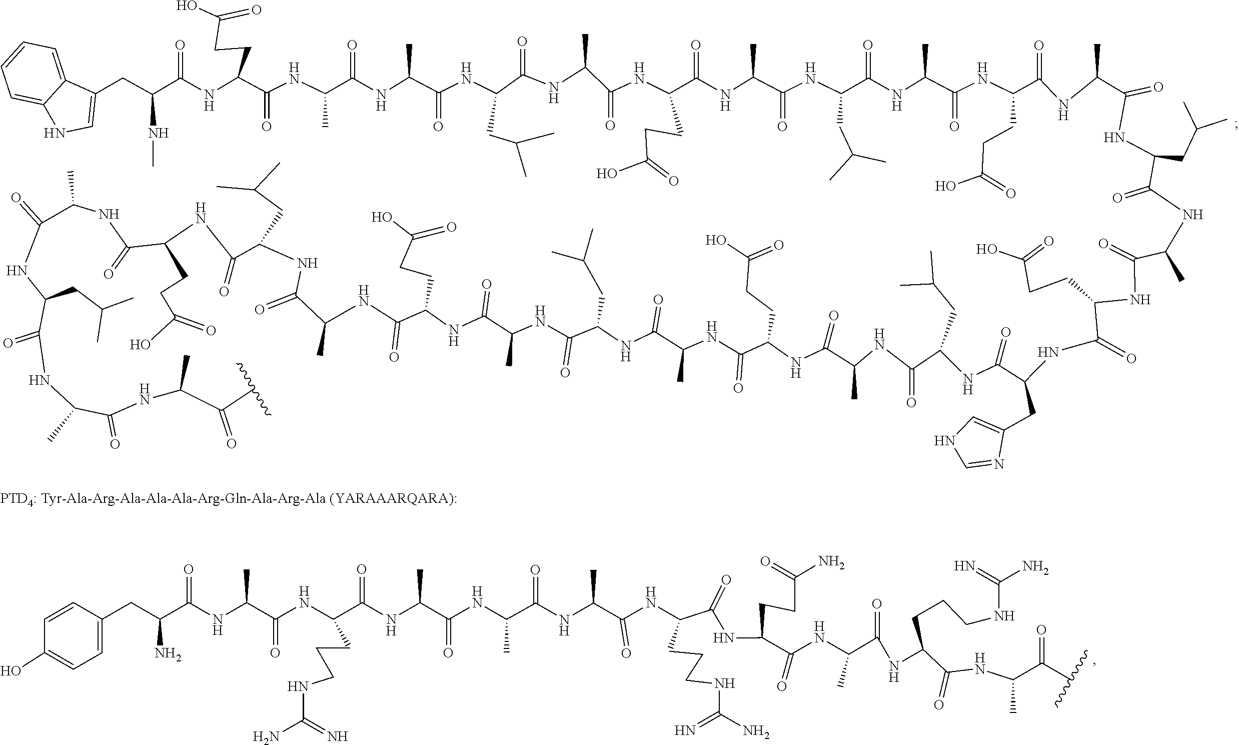

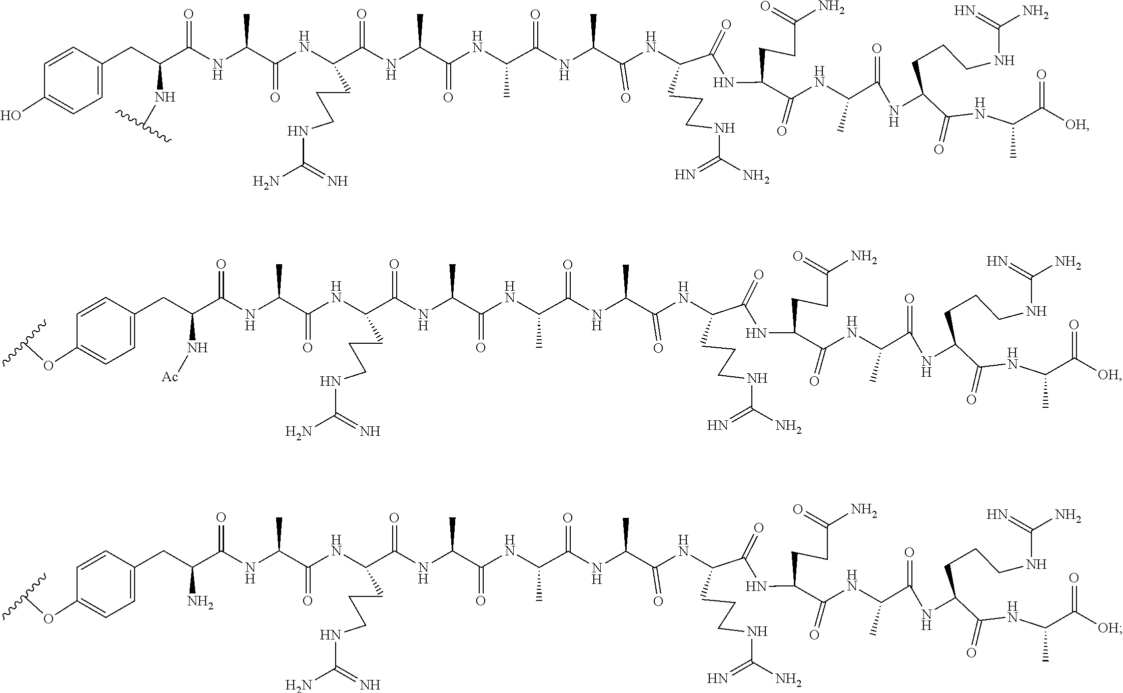

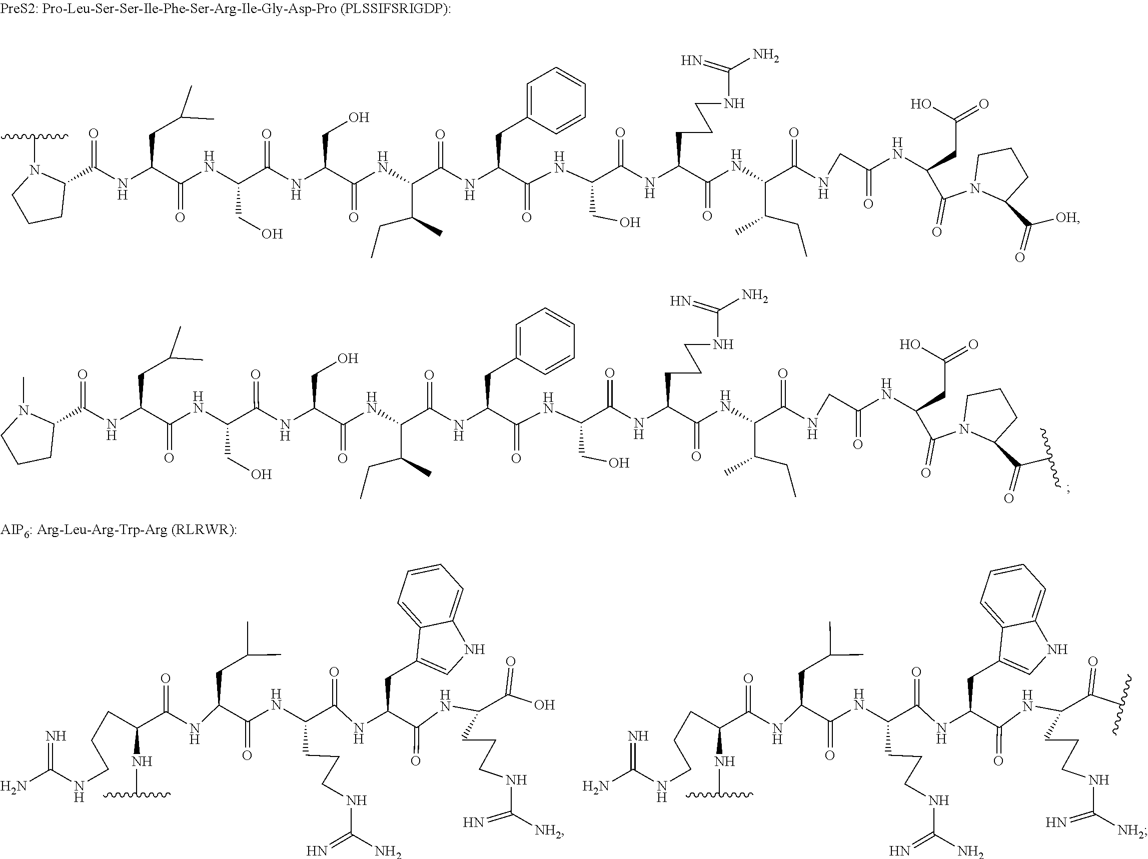

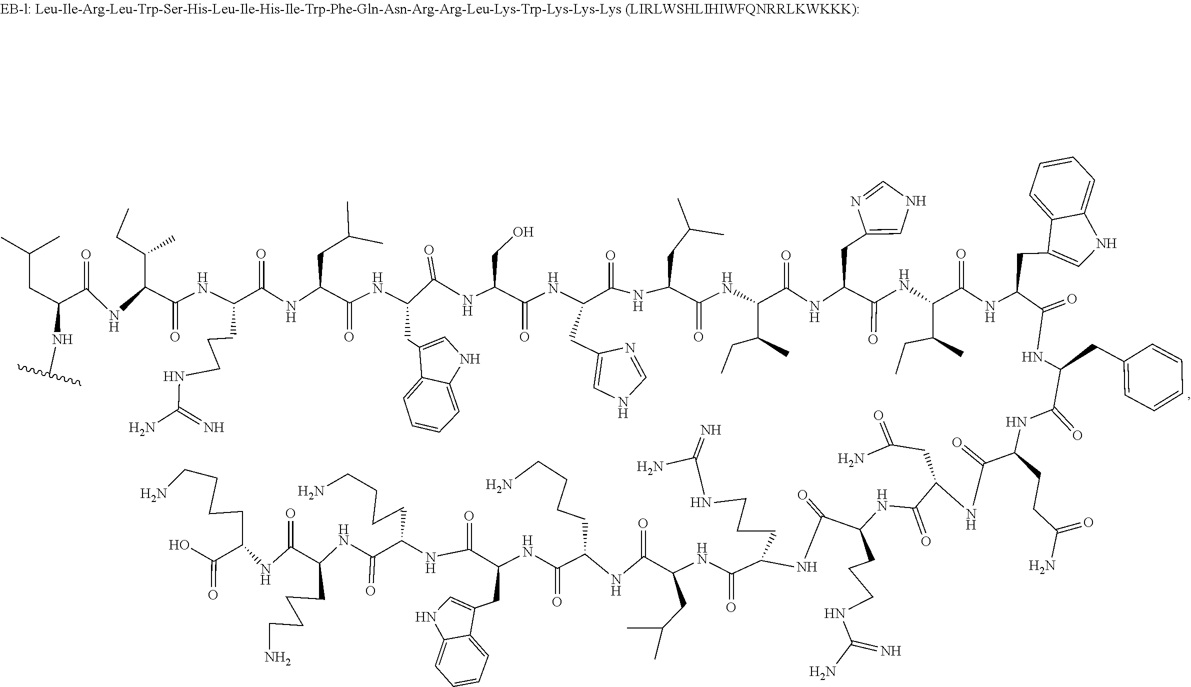

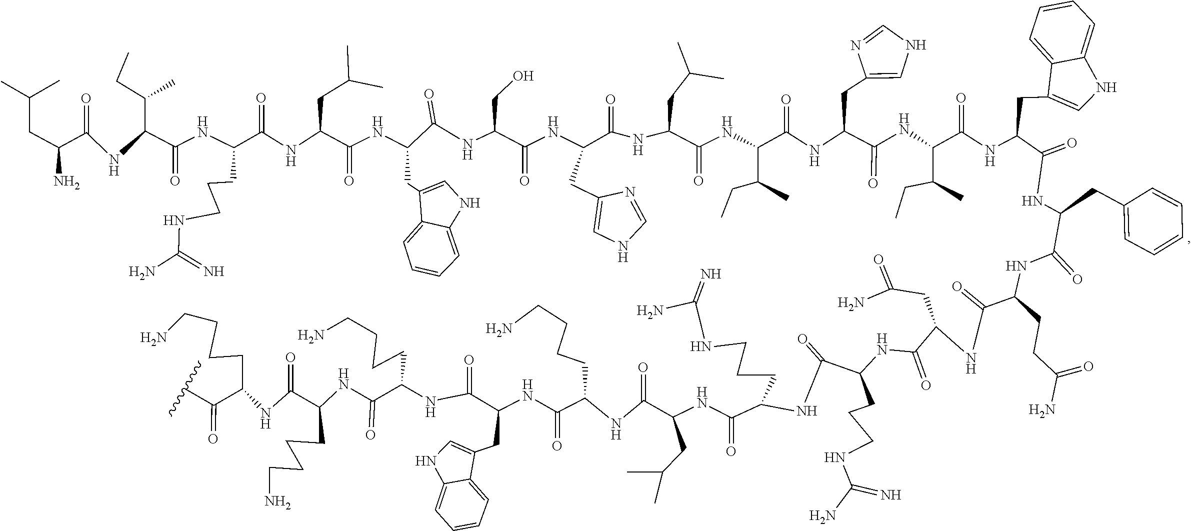

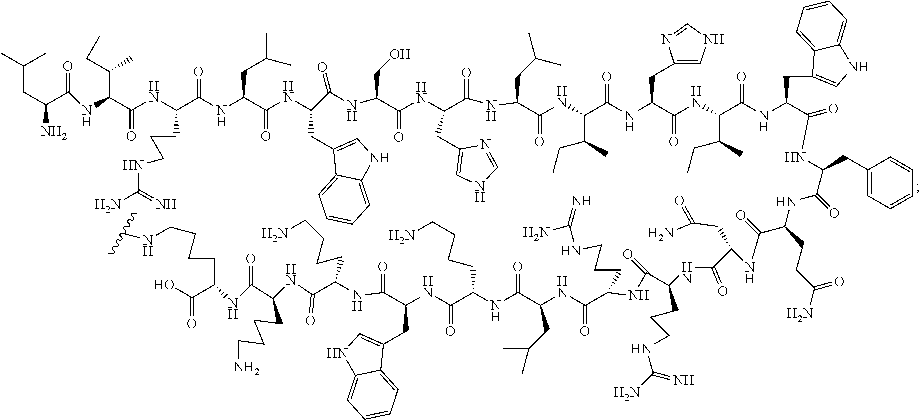

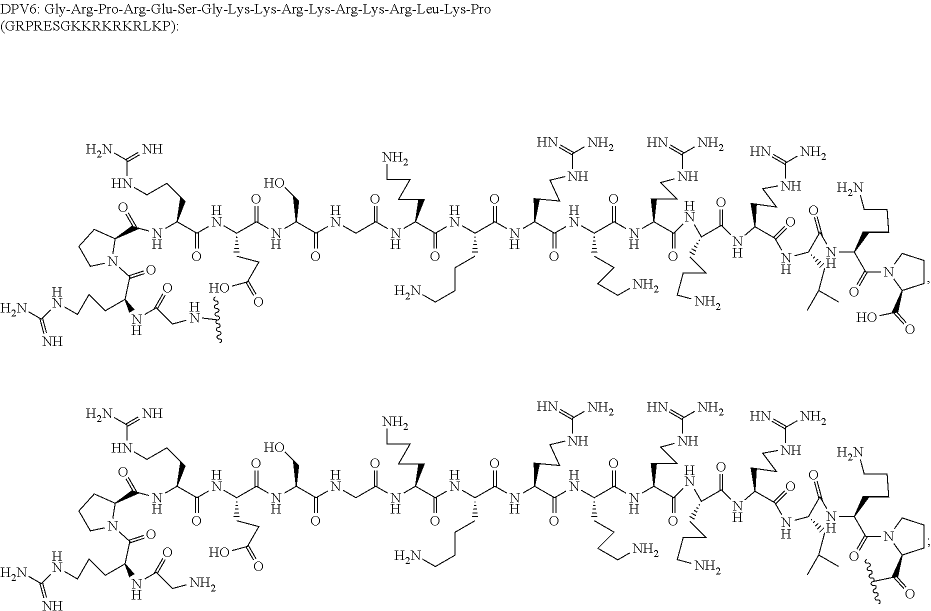

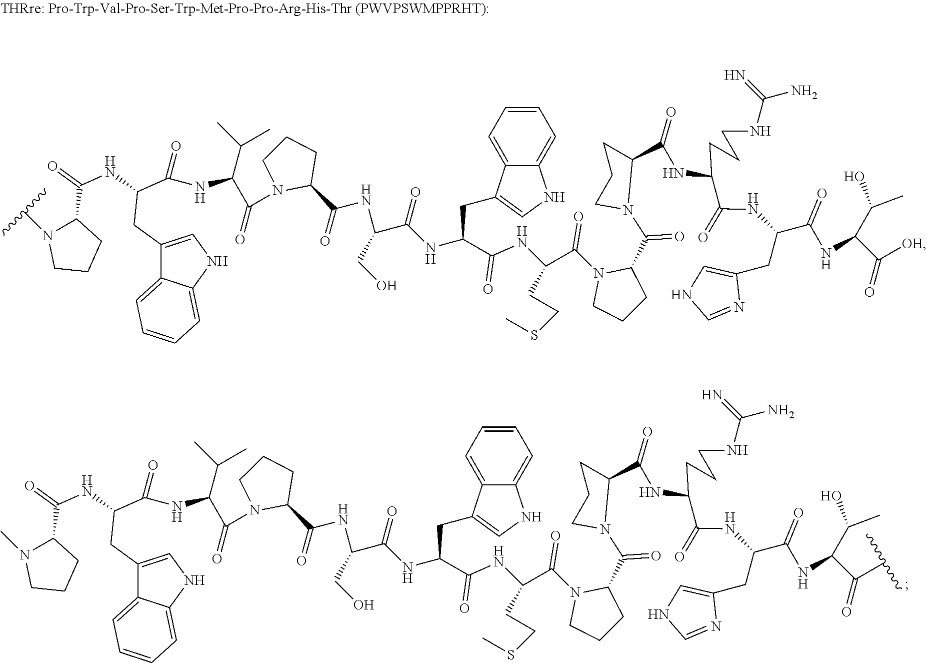

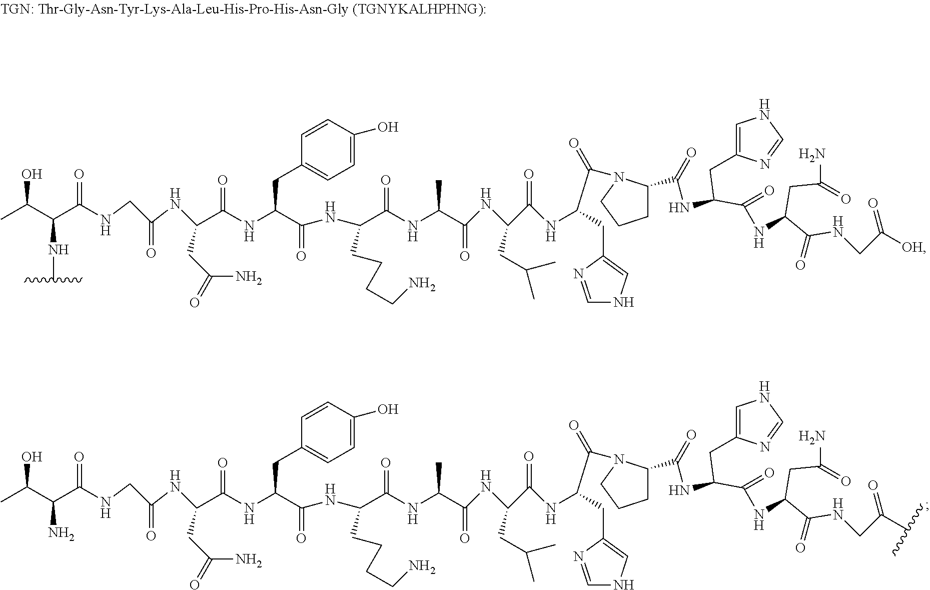

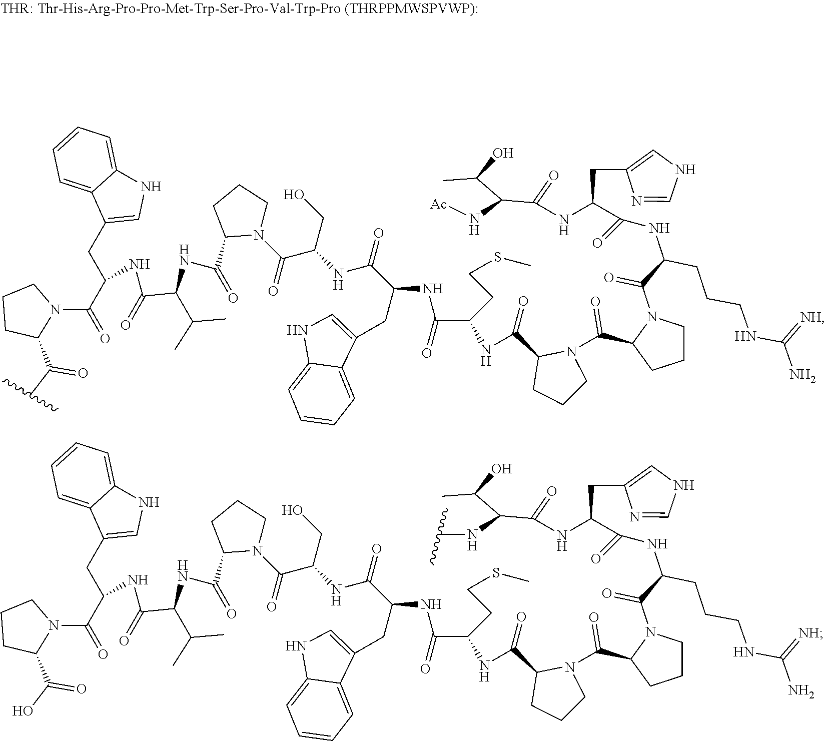

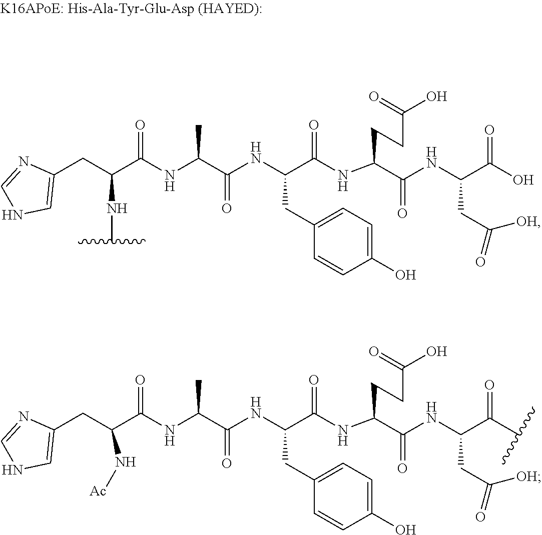

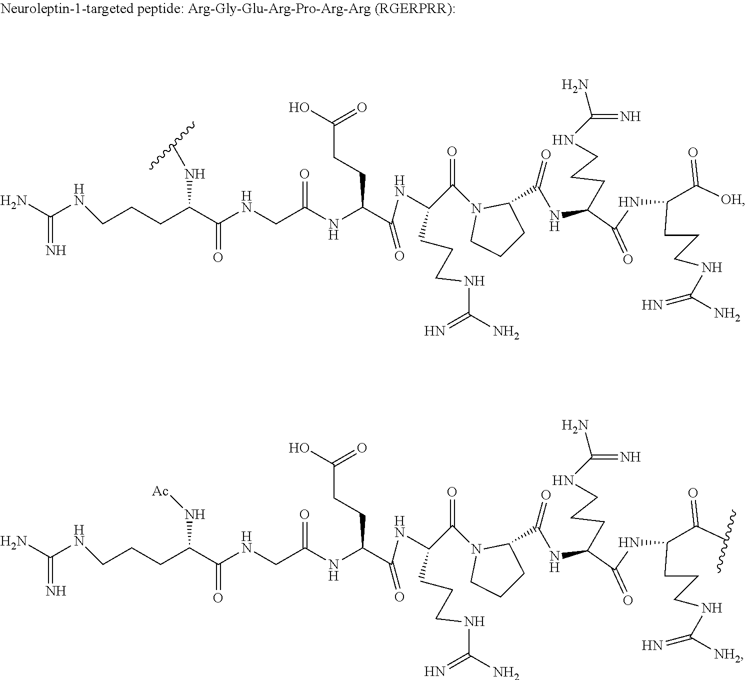

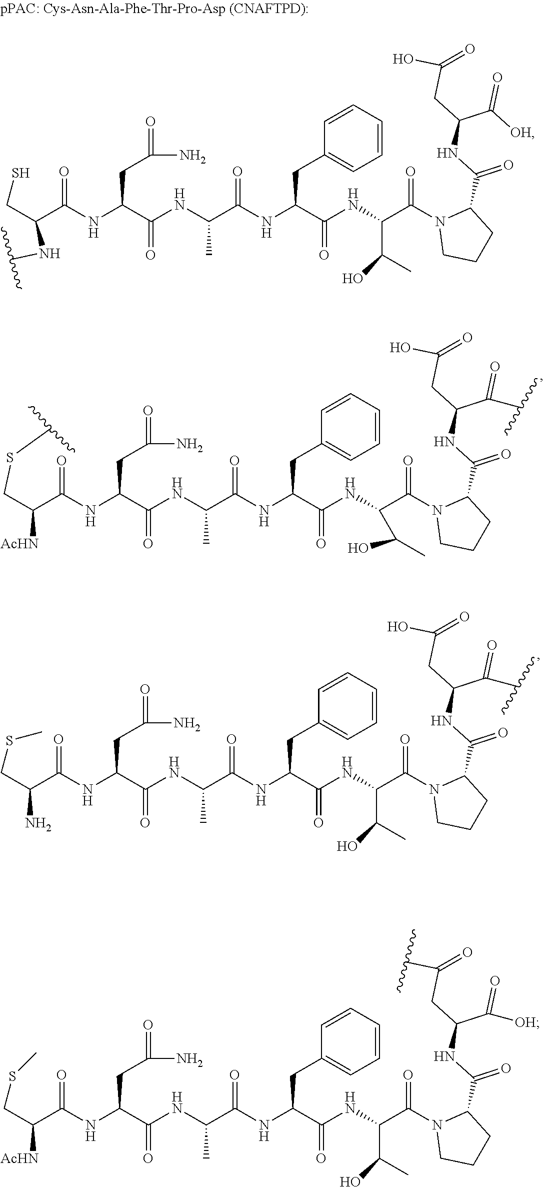

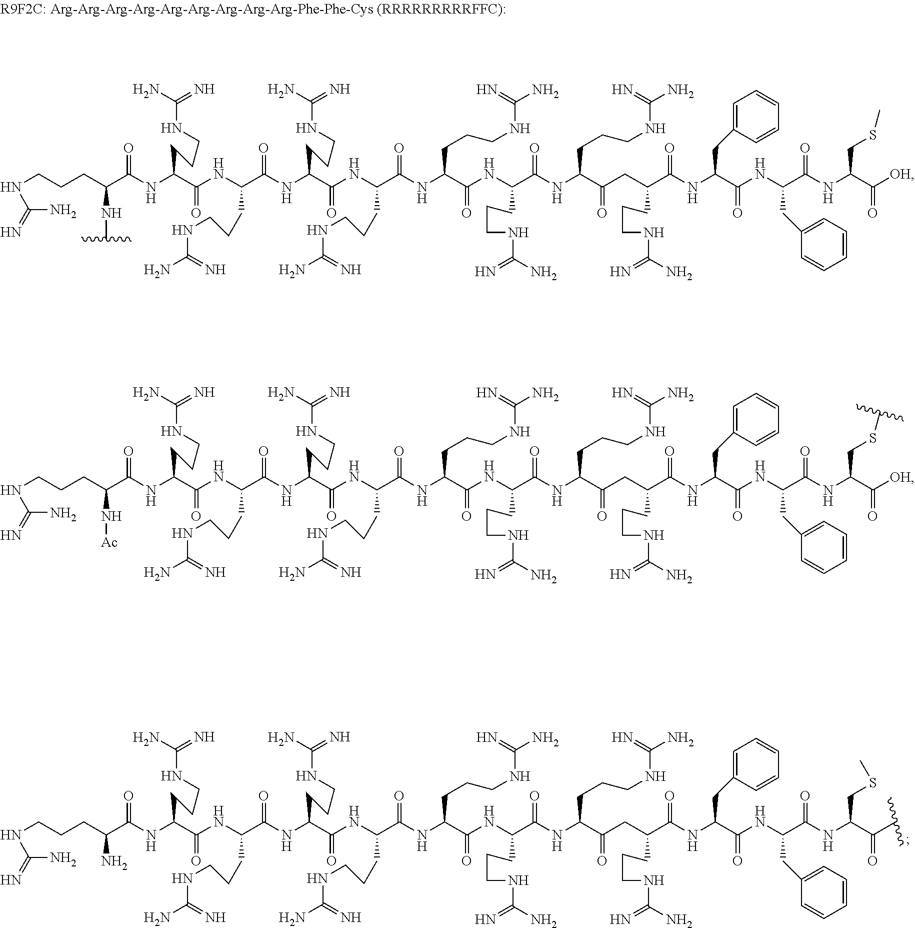

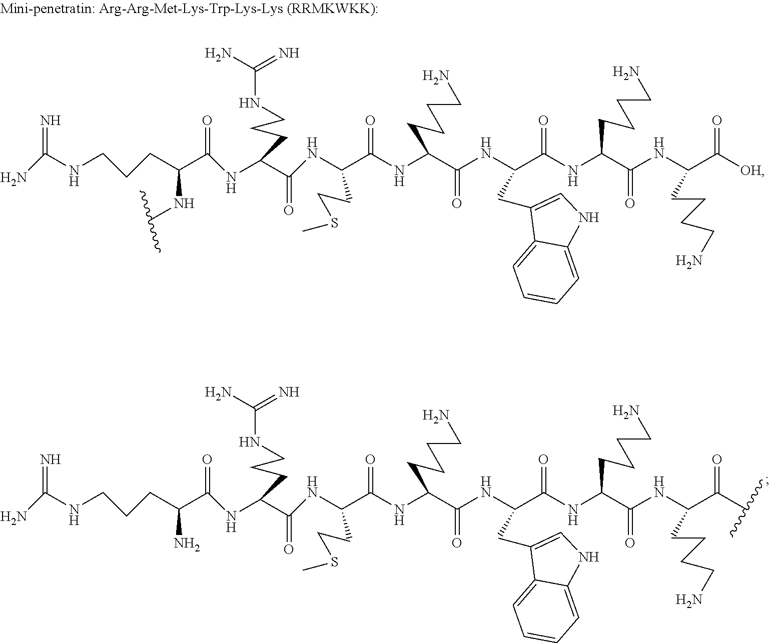

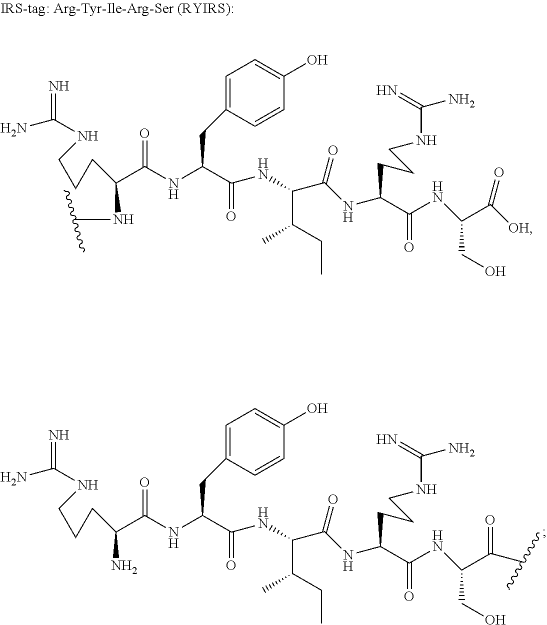

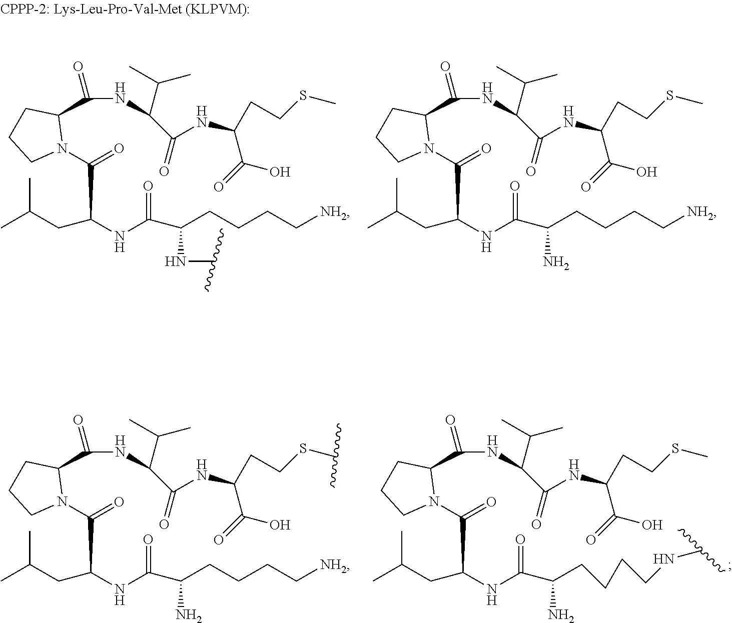

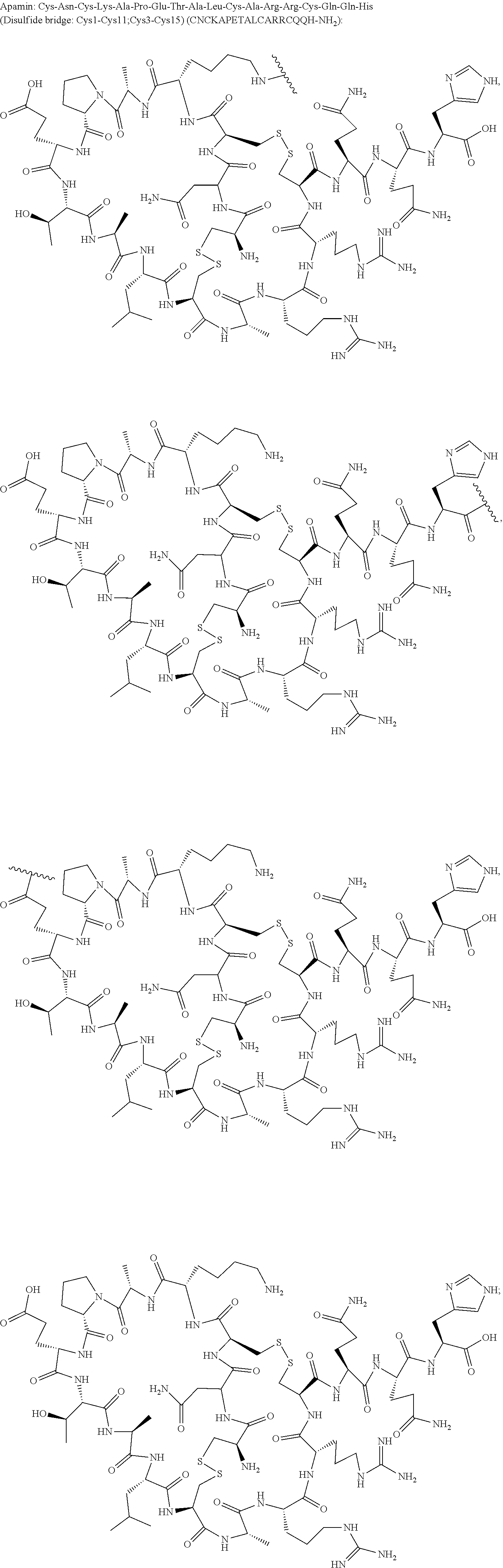

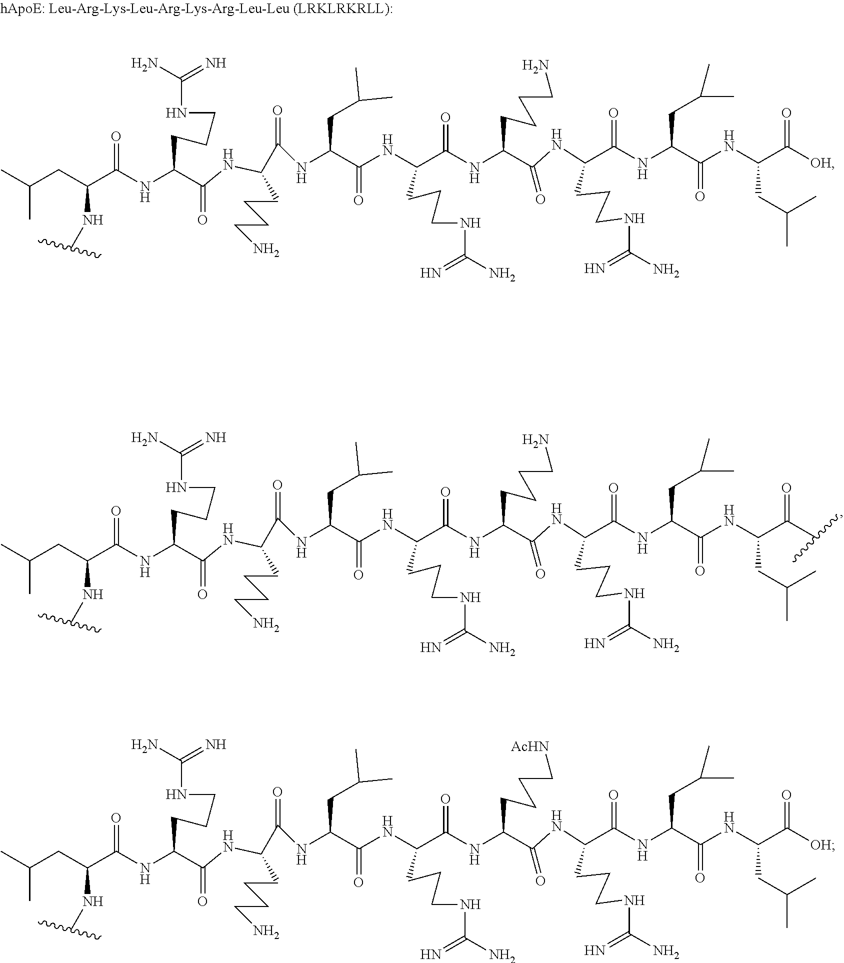

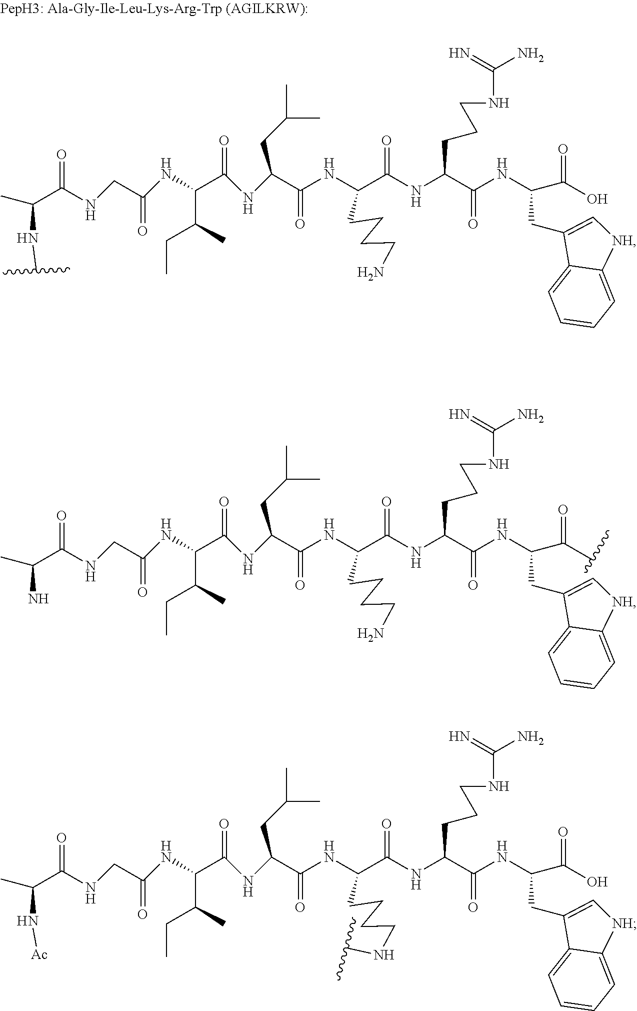

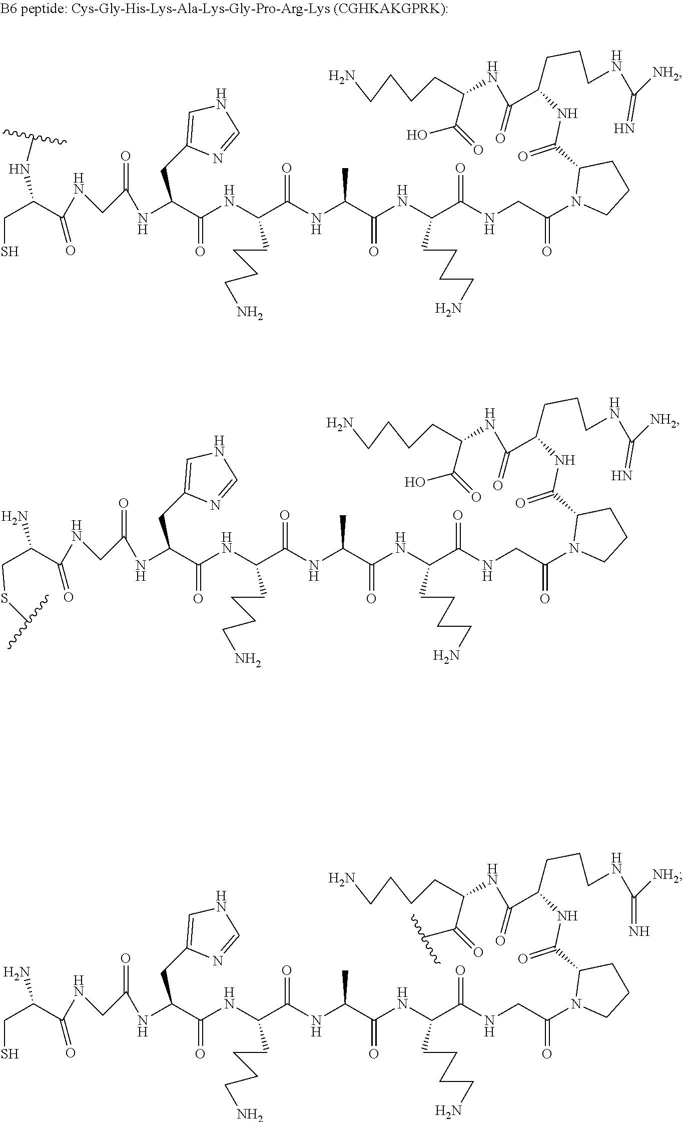

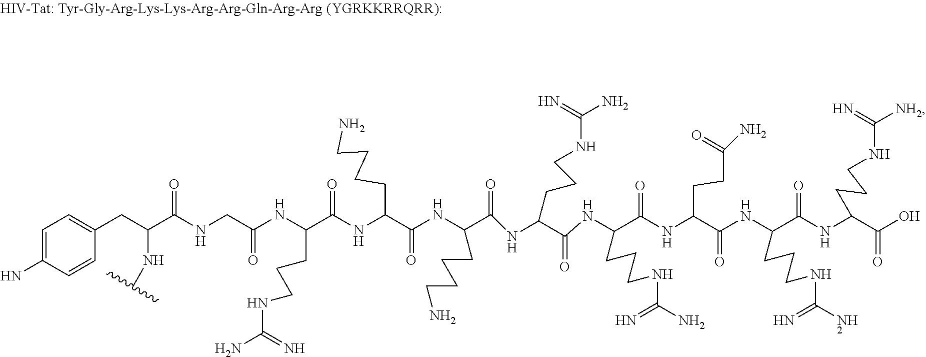

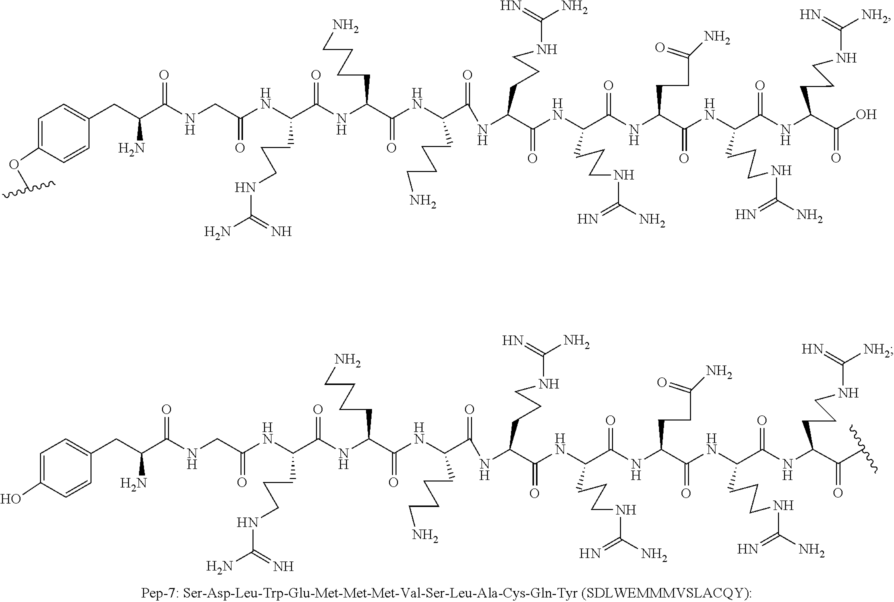

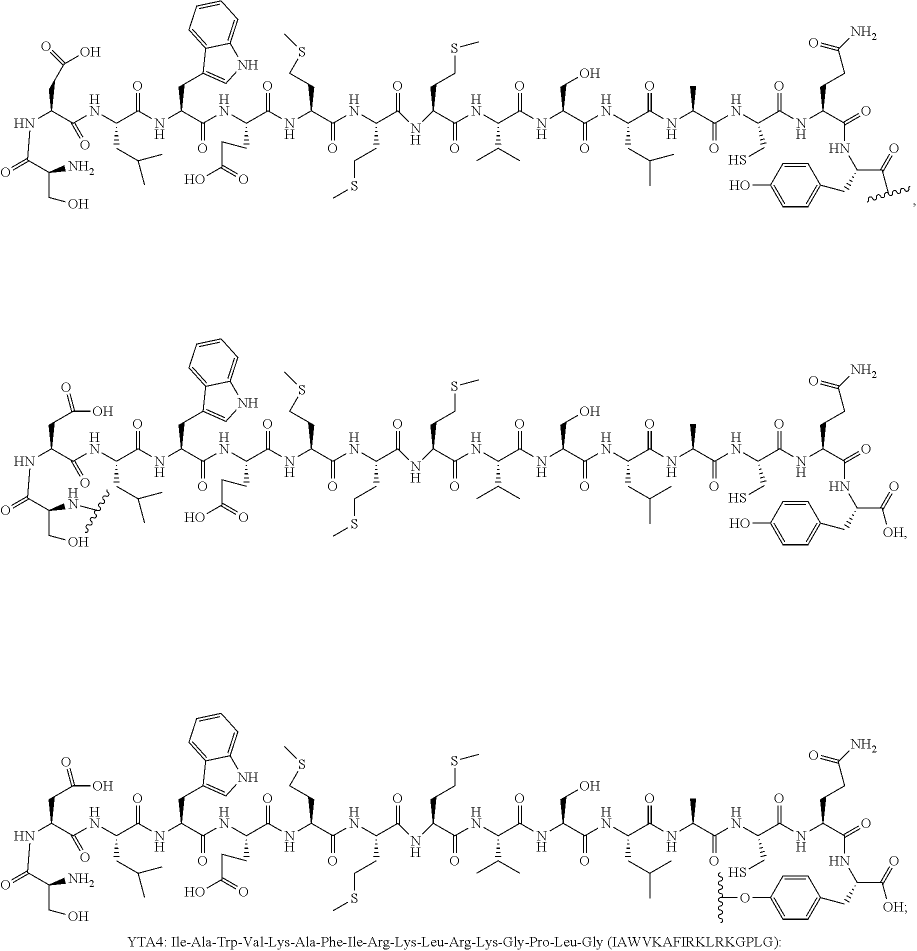

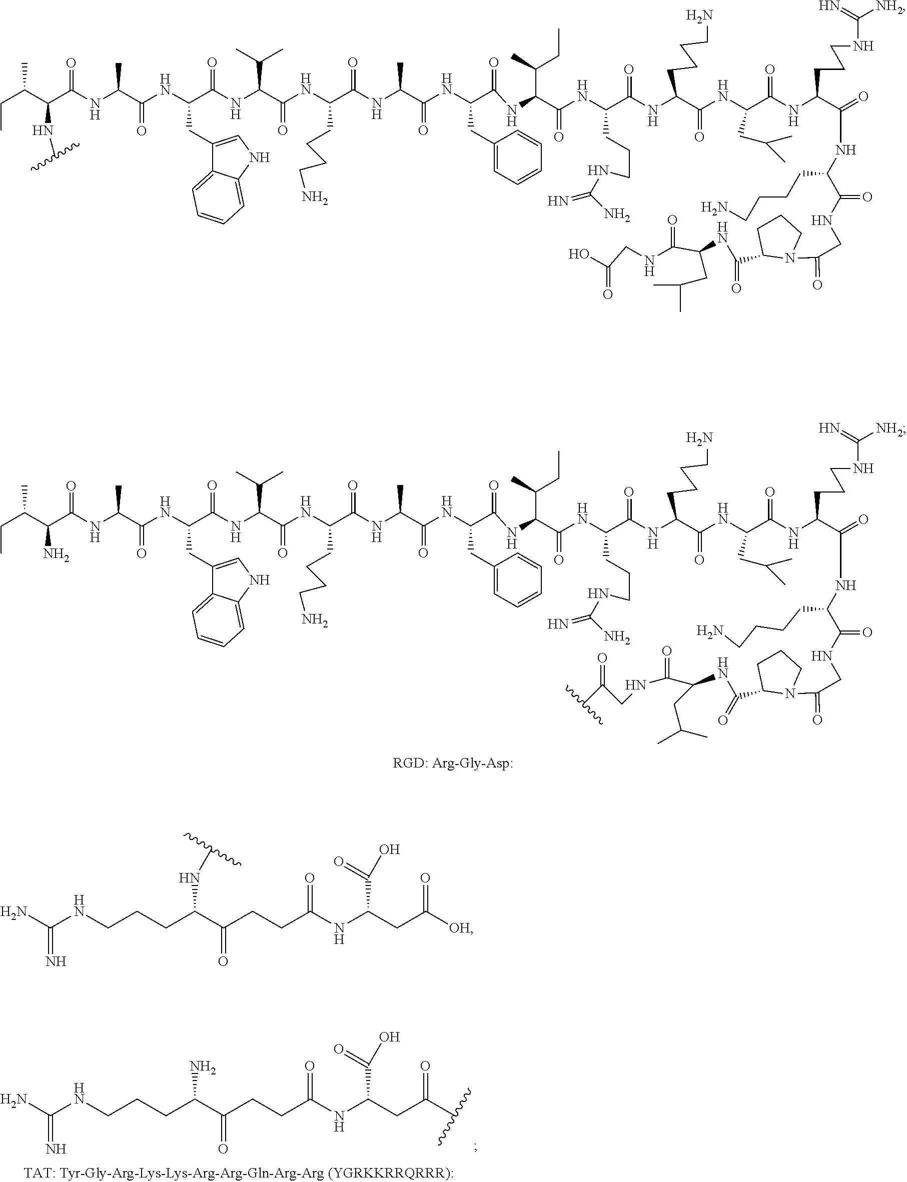

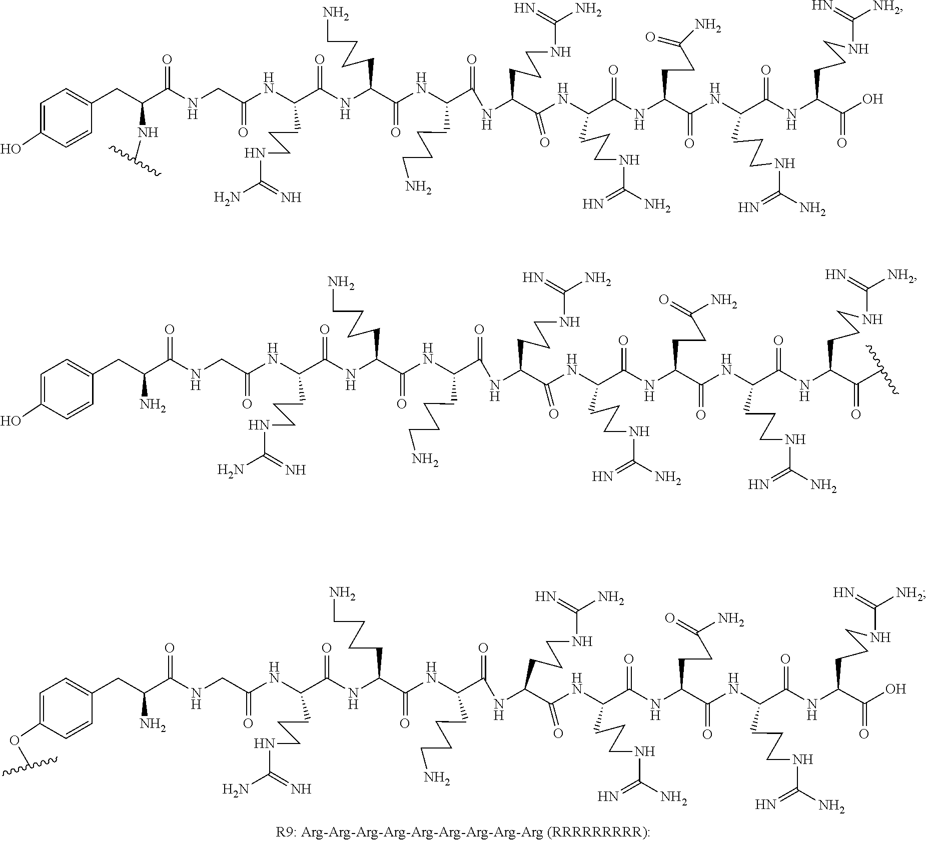

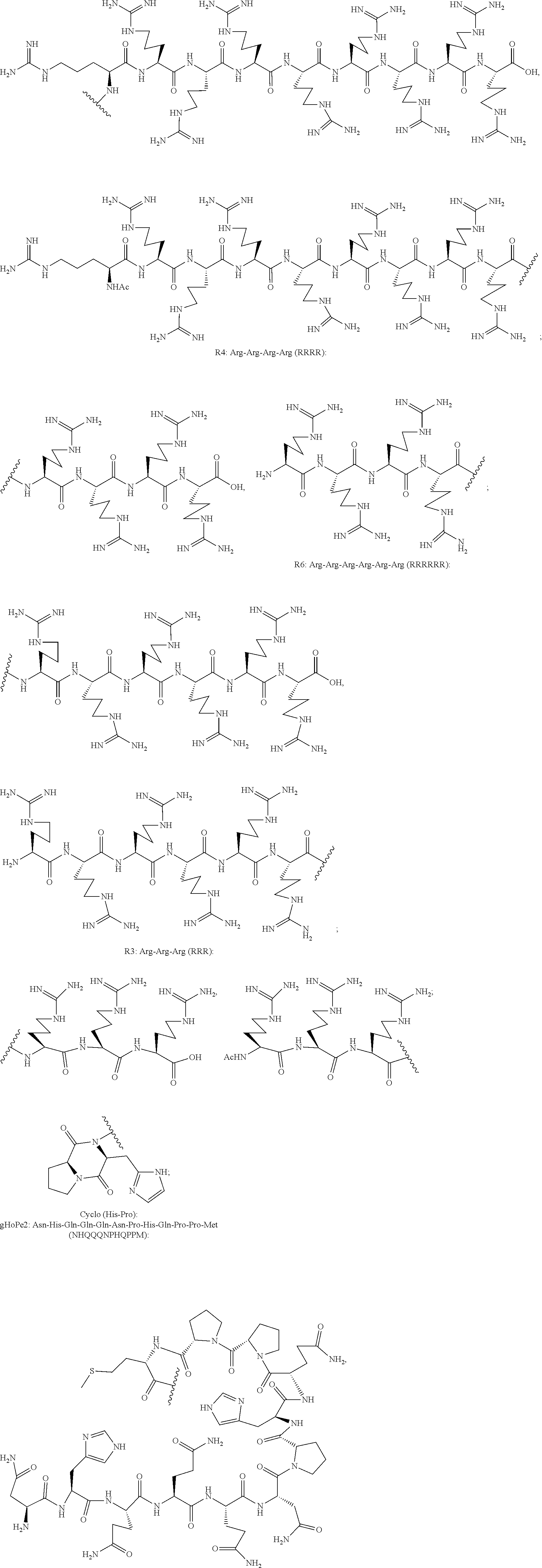

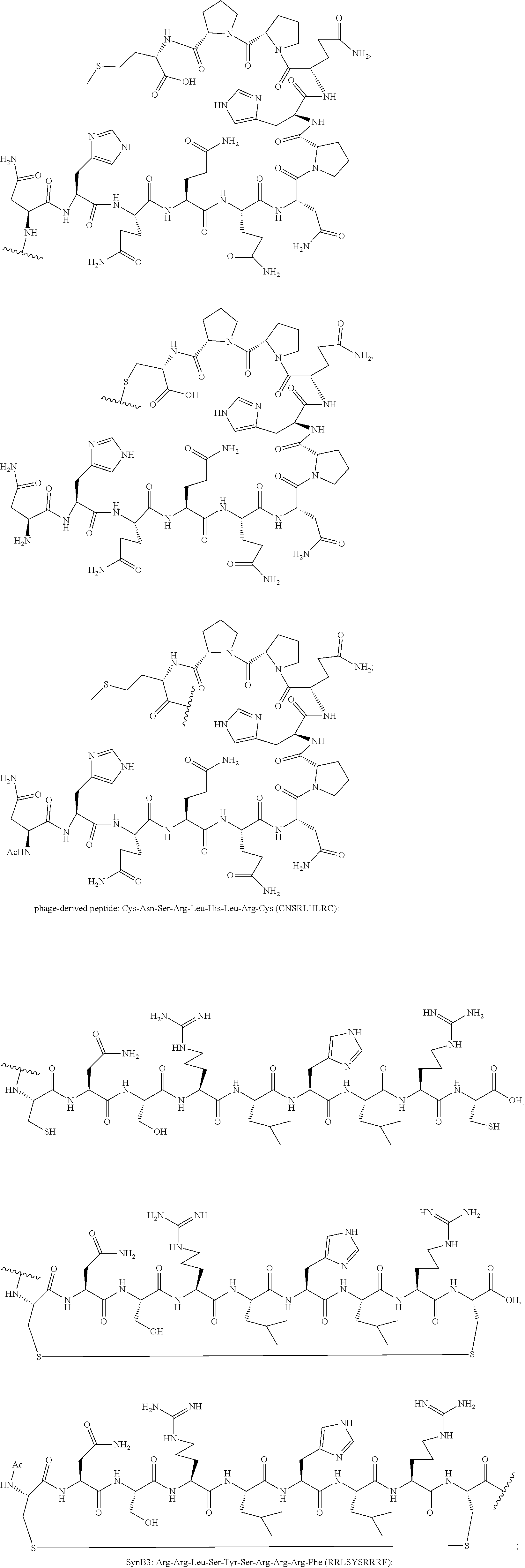

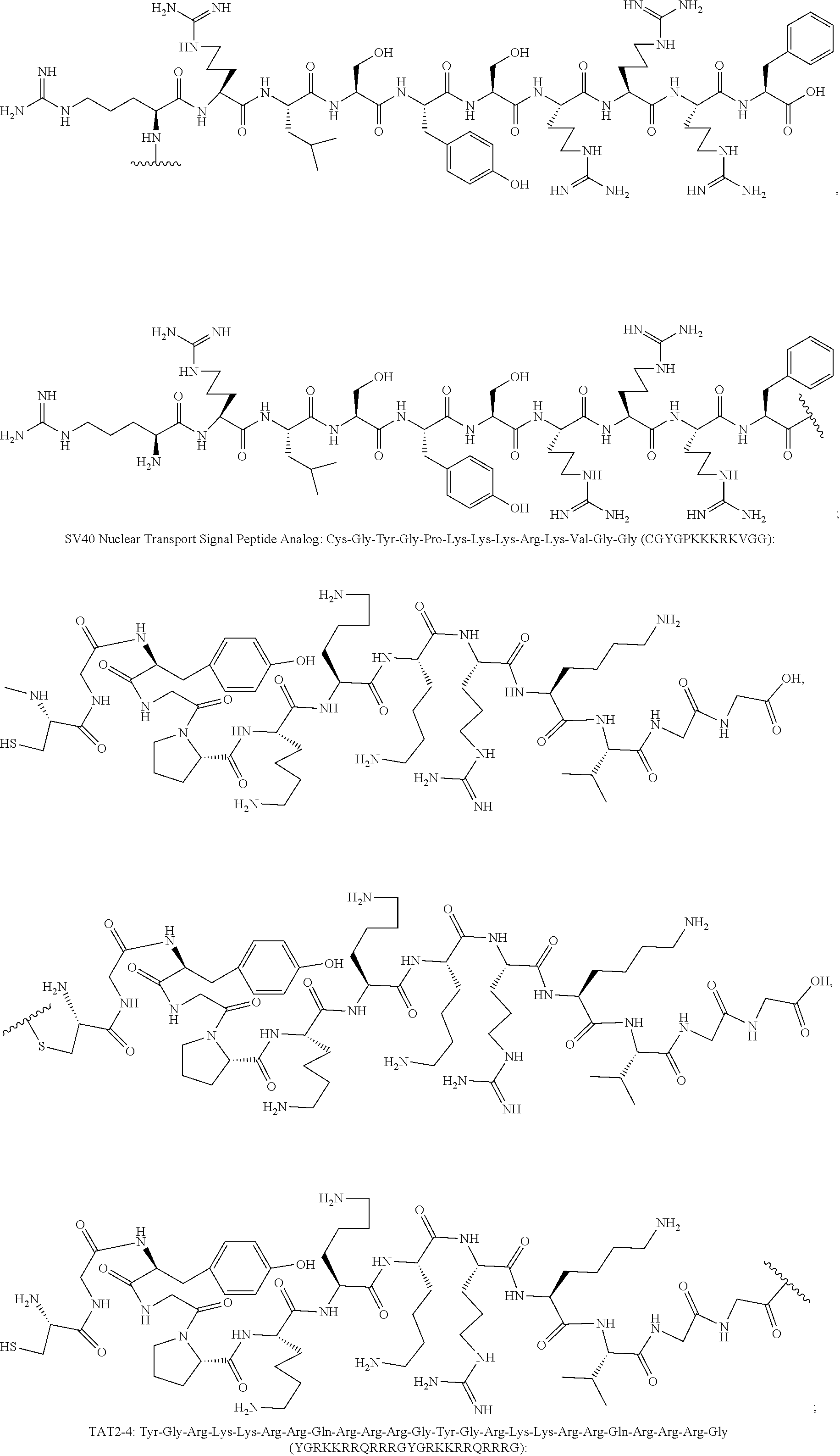

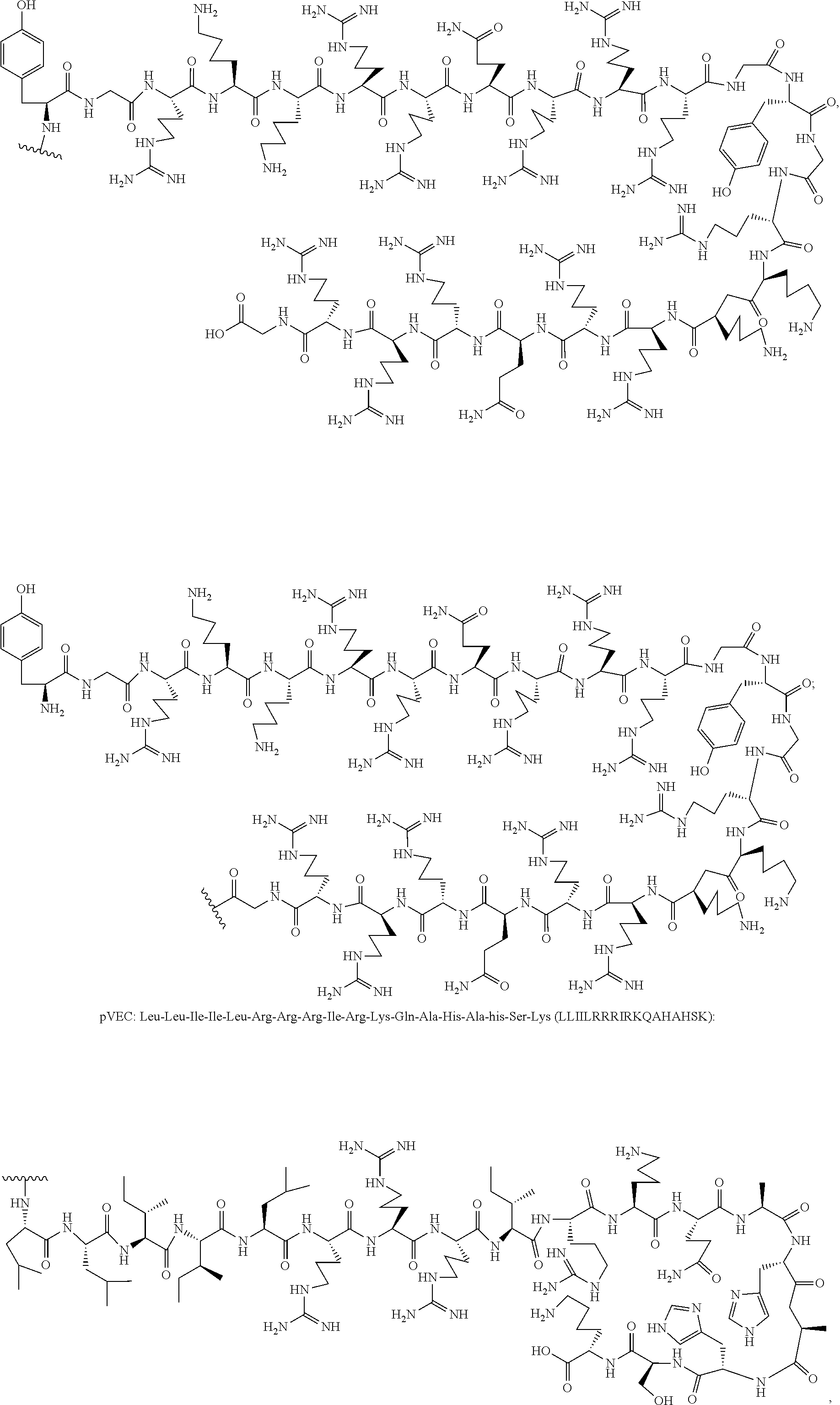

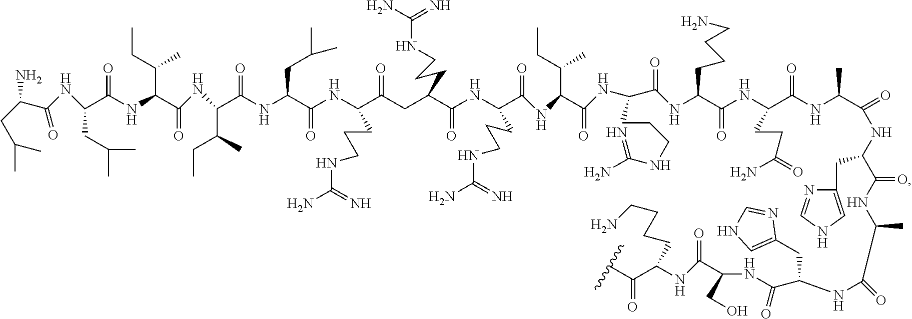

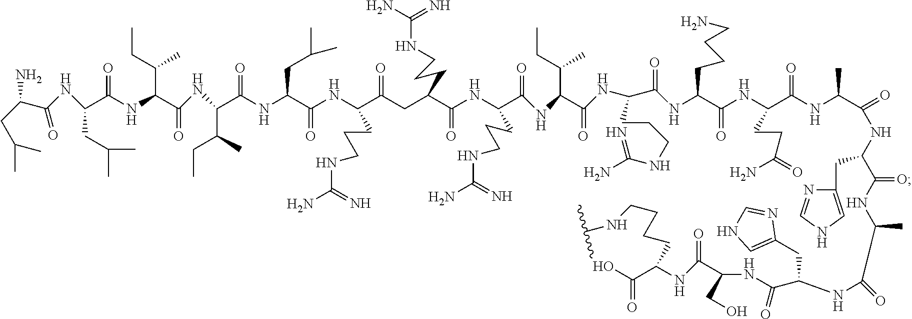

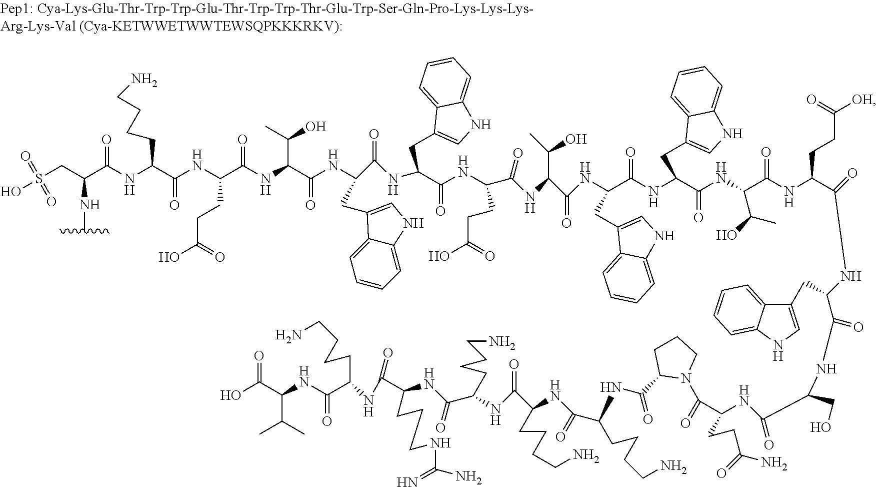

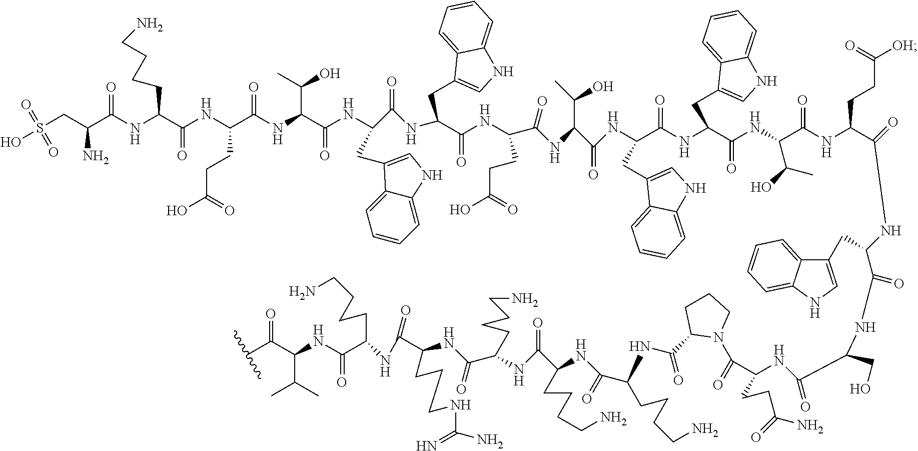

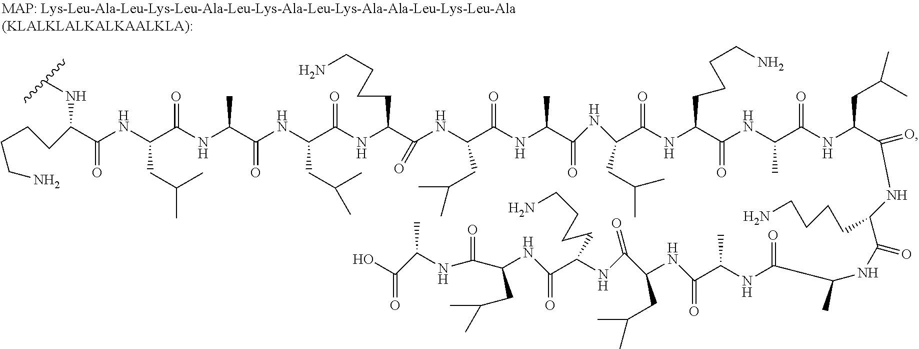

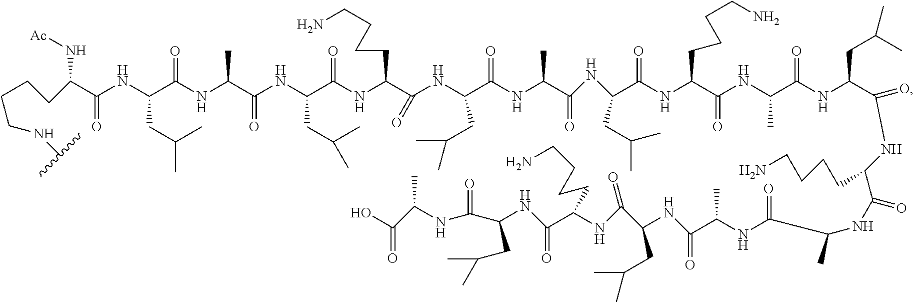

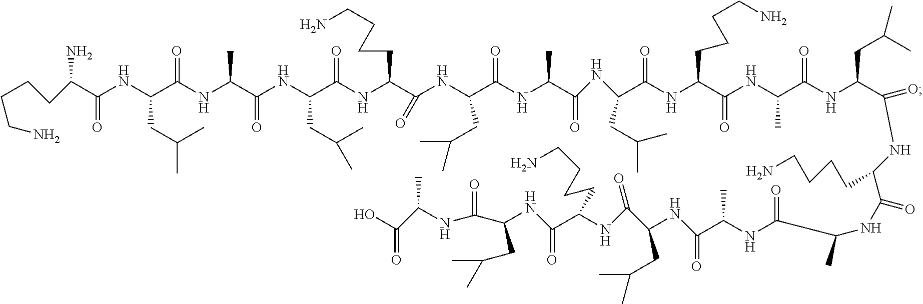

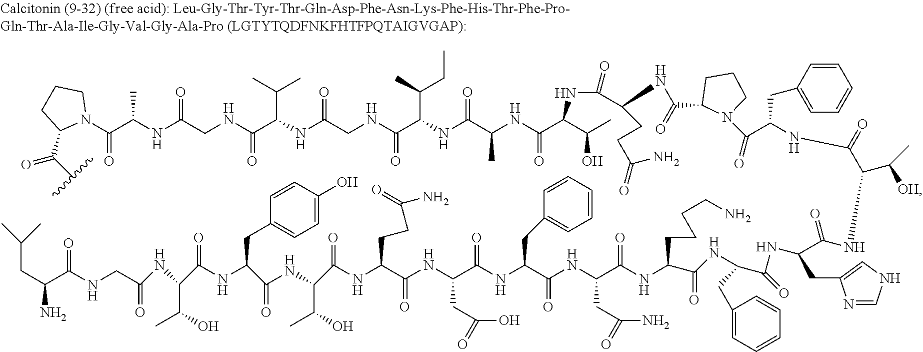

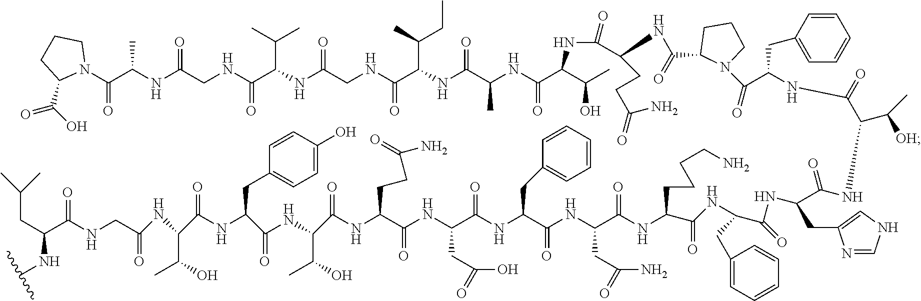

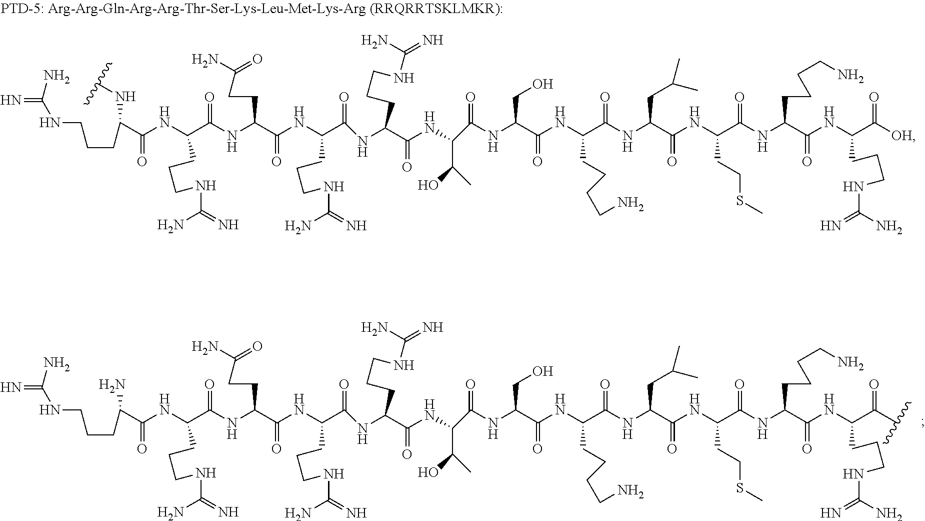

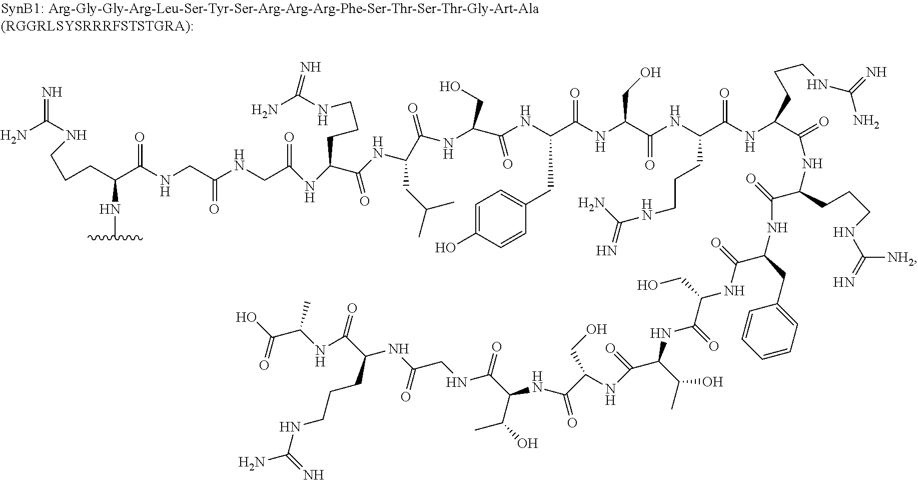

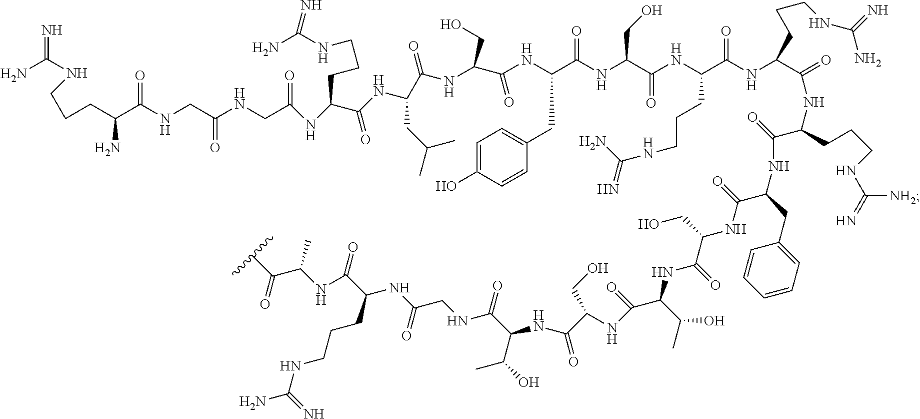

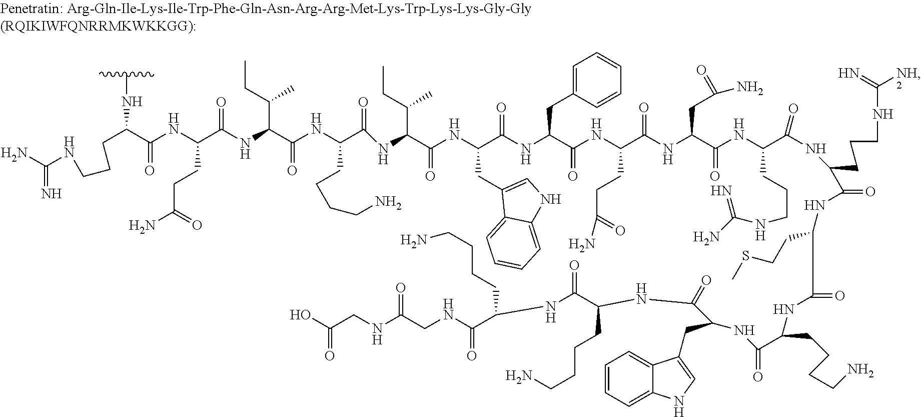

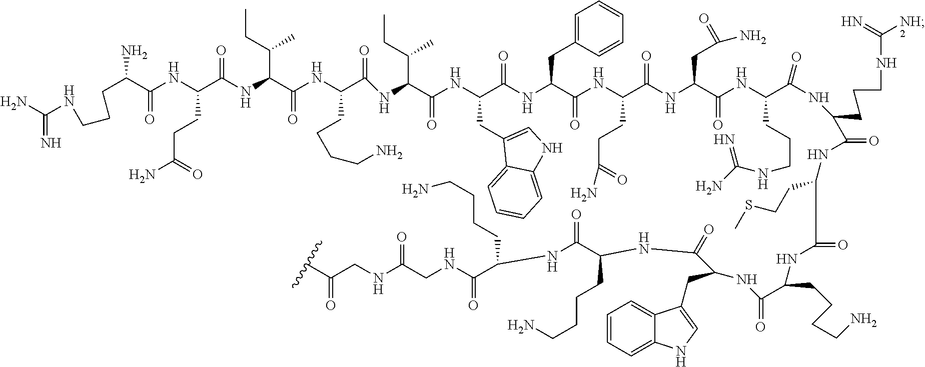

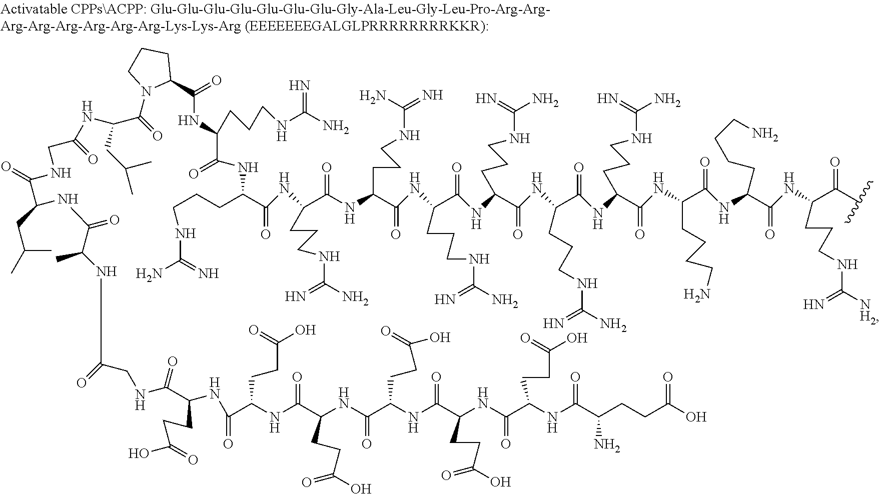

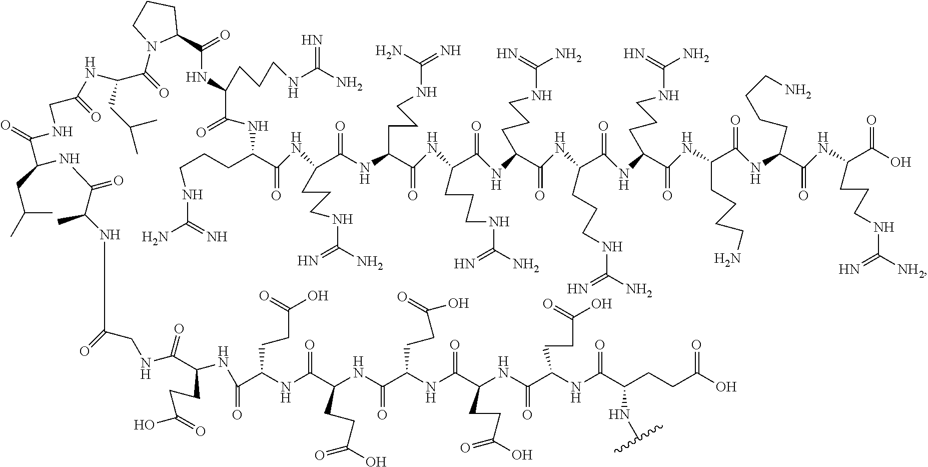

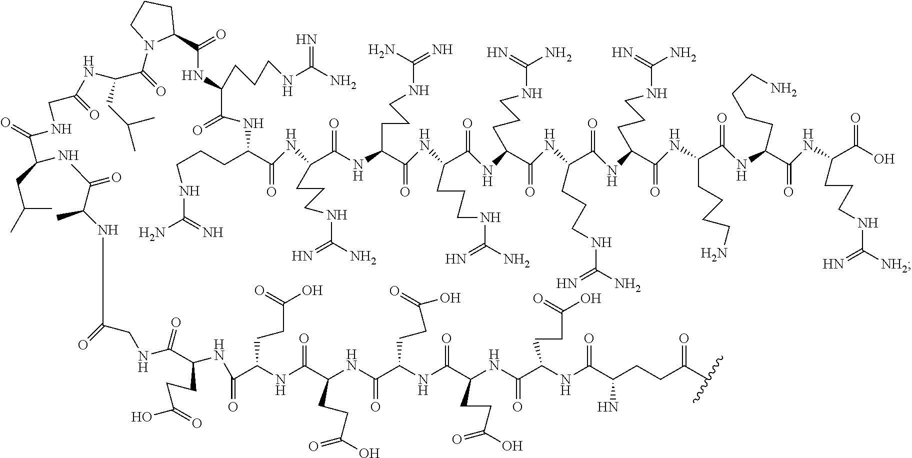

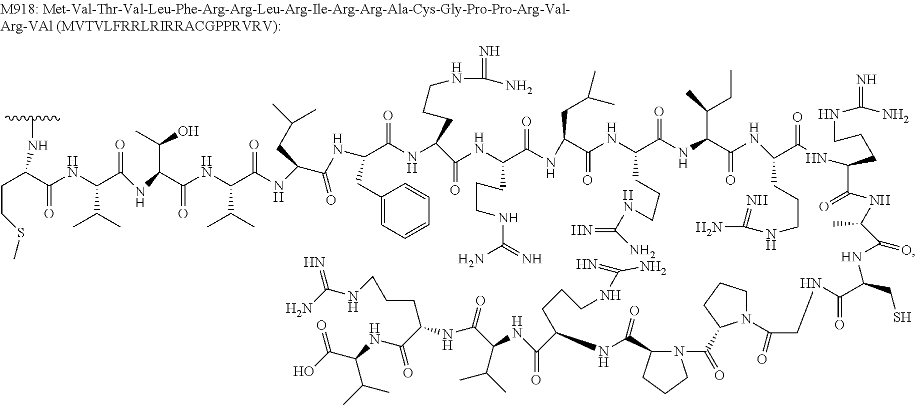

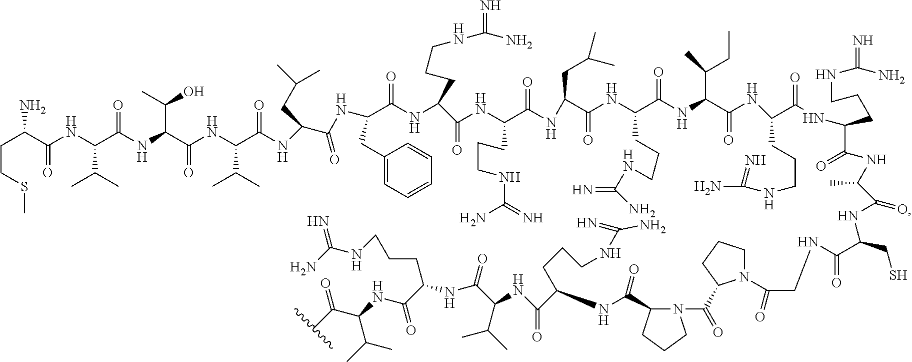

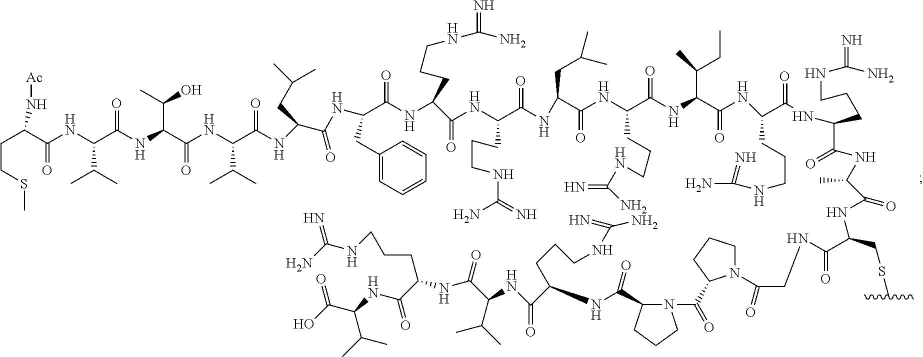

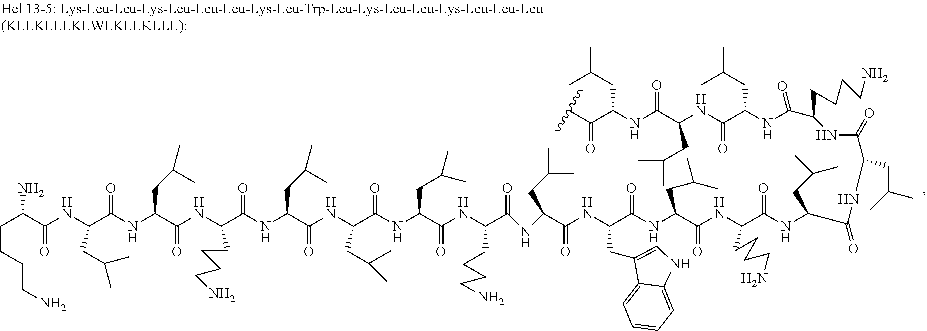

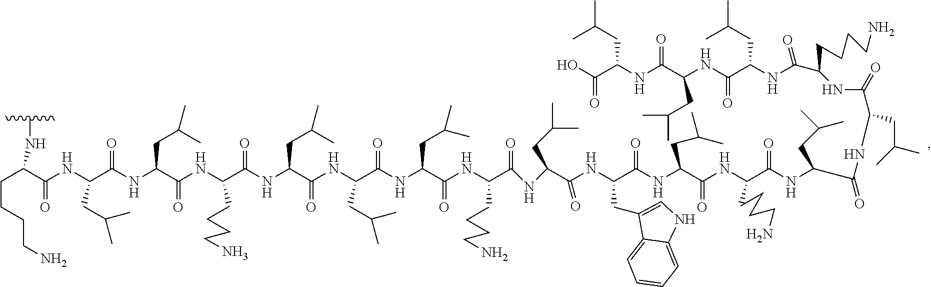

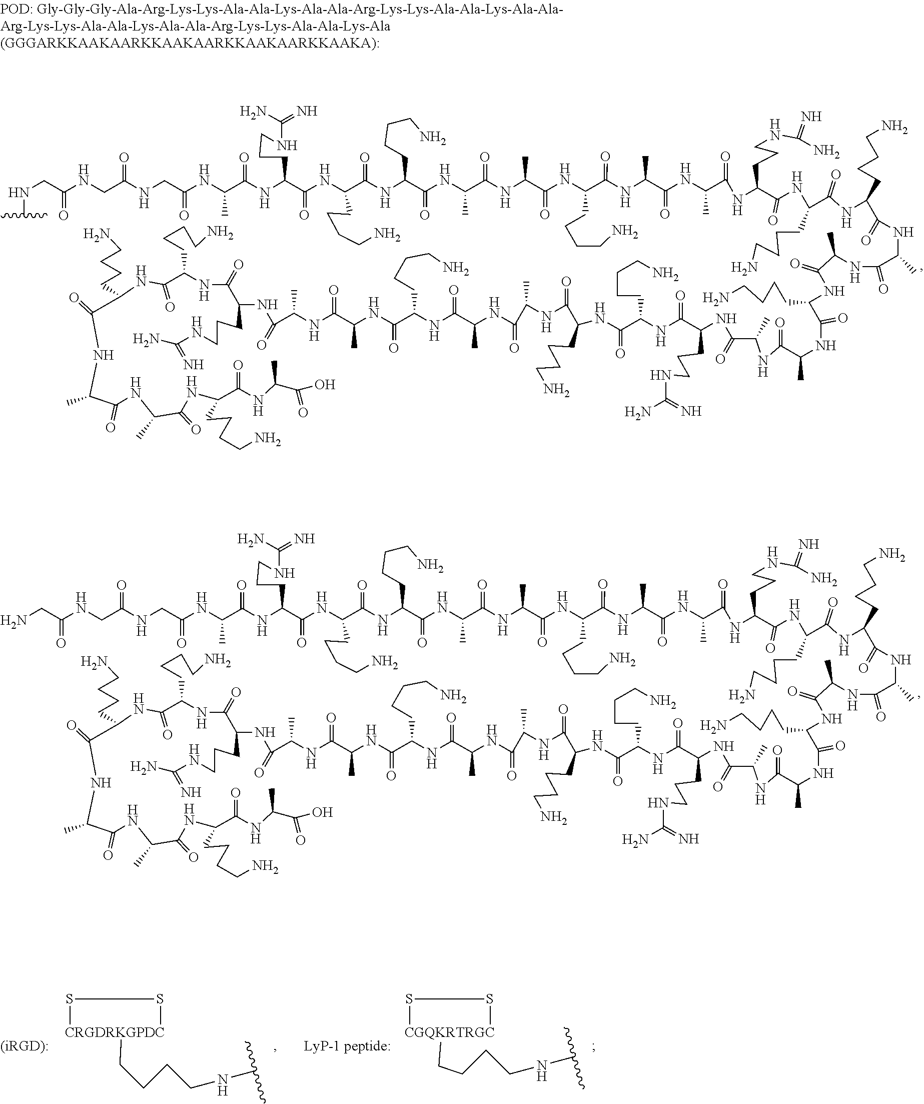

wherein [symbol] is the site linked to Ld₁, Ld₂, Ld₃, Ld₄, Ld₅, or Ld₆; Ra is Ar, which is preferably selected from

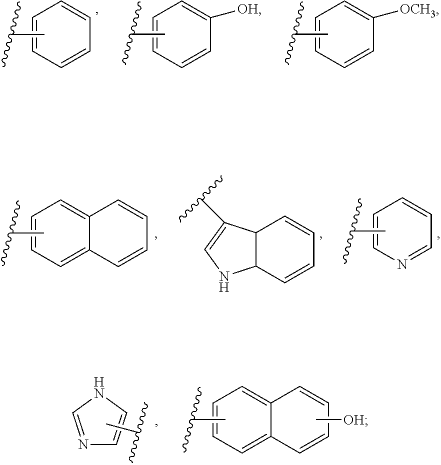

Rb is OH, COOH, COOCH₃, CH₃OH, CH₃NH₂, CONH₂;

In another embodiments, A₁, A₂, A₃, A₄, A₅ and A₆ are Nucleoside analogues, which have synergy with D₁ or/and D₂.

Nucleoside analogues: they are molecules that act like nucleosides in DNA synthesis. They include a range of antiviral products used to prevent viral replication in infected cells. Nucleoside analogues can be used against hepatitis B virus, hepatitis C virus, herpes simplex, and HIV. Once they are phosphorylated, they work as antimetabolites by being similar enough to nucleotides to be incorporated into growing DNA strands. Less selective nucleoside analogues are used as chemotherapy agents to treat cancer, e.g. gemcitabine and 5-FU. Antimetabolite is a chemical that inhibits the use of a metabolite, which is another chemical that is part of normal metabolism. Such substances are often similar in structure to the metabolite that they interfere with, such as the antifolates that interfere with the use of folic acid. The presence of antimetabolites can have toxic effects on cells, such as halting cell growth and cell division, so these compounds are used as chemotherapy for cancer. Some conjugatable structures of nucleoside analogues for the patent are illustrated below:

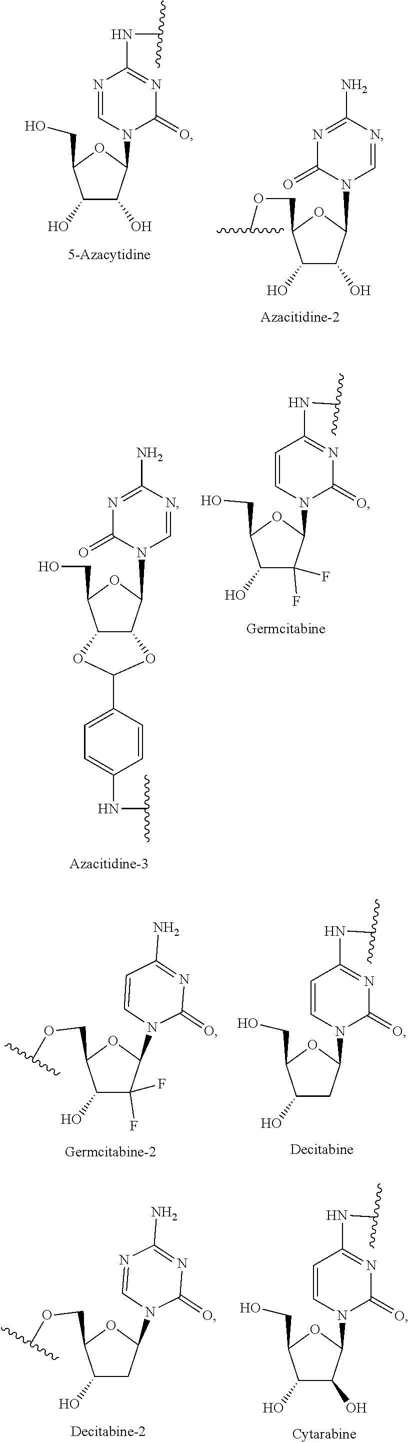

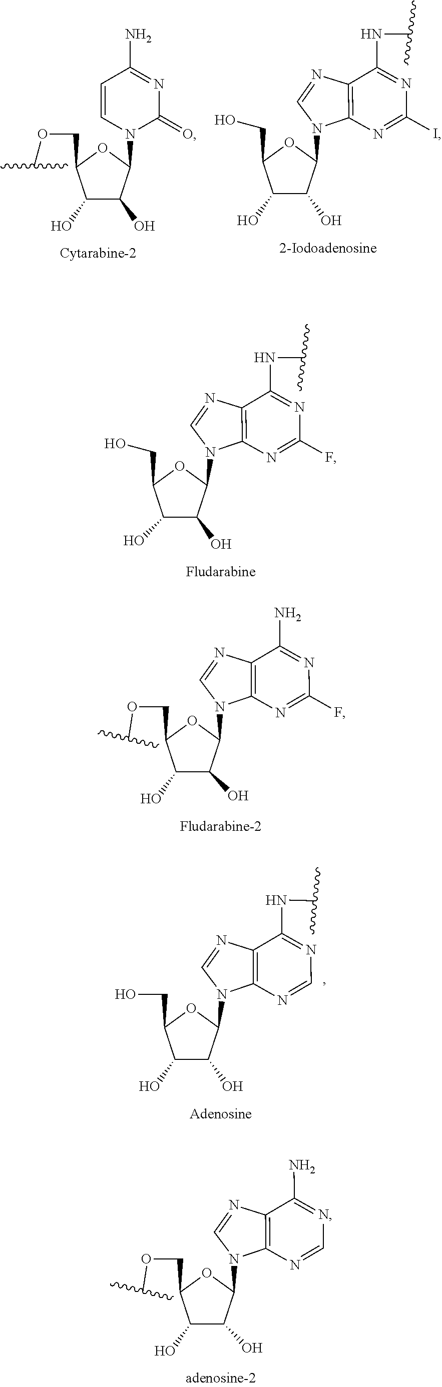

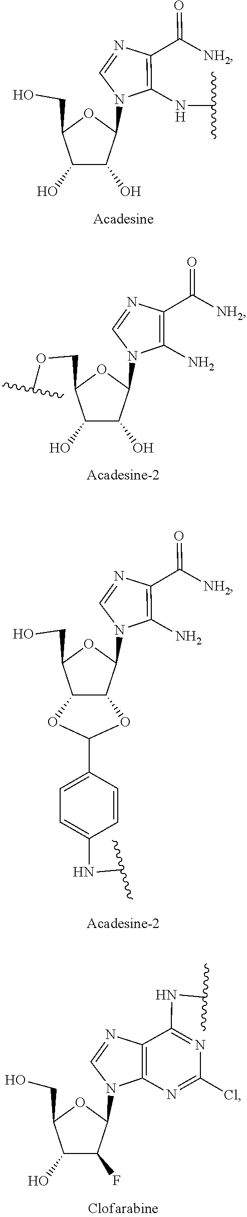

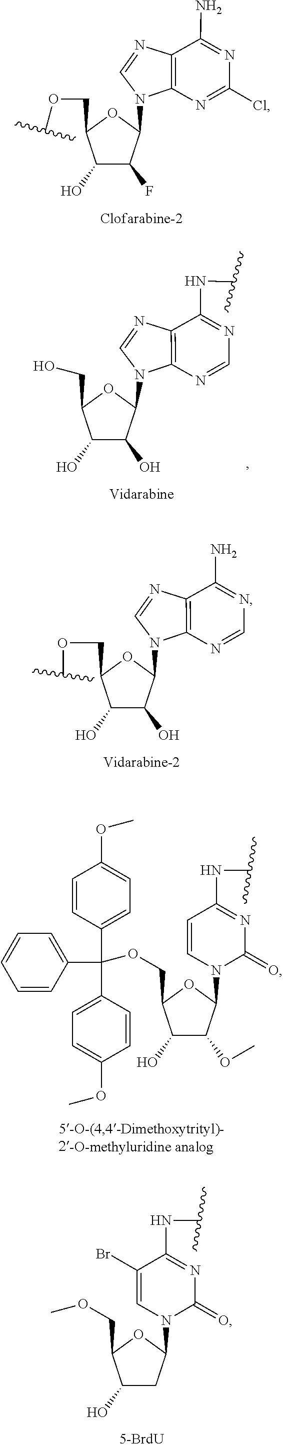 [SMILES: *OC[C@H]1O[C@@H](n2cnc3c(N)nc(Cl)nc32)[C@@H](F)[C@@H]1O ; *Nc1ncnc2c1ncn2[C@@H]1O[C@H](CO)[C@@H](O)[C@@H]1O ; *OC[C@H]1O[C@@H](n2cnc3c(N)ncnc32)[C@@H](O)[C@@H]1O ; *Nc1ccn([C@@H]2O[C@H](COC(c3ccccc3)(c3ccc(OC)cc3)c3ccc(OC)cc3)[C@@H](O)[C@H]2OC)c(=O)n1 ; *Nc1nc(=O)n([C@H]2C[C@H](O)[C@@H](COC)O2)cc1Br]

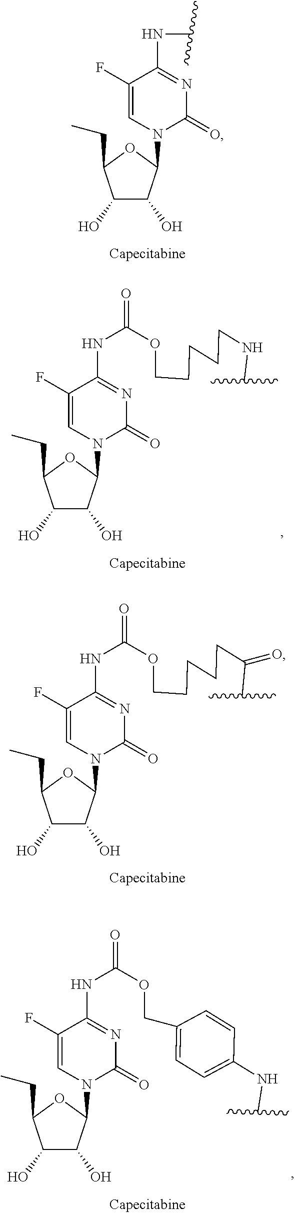 [SMILES: *Nc1nc(=O)n([C@@H]2O[C@H](CC)[C@@H](O)[C@H]2O)cc1F ; *NCCCCCOC(=O)Nc1nc(=O)n([C@@H]2O[C@H](CC)[C@@H](O)[C@H]2O)cc1F ; *C(=O)CCCCCOC(=O)Nc1nc(=O)n([C@@H]2O[C@H](CC)[C@@H](O)[C@H]2O)cc1F ; *Nc1ccc(COC(=O)Nc2nc(=O)n([C@@H]3O[C@H](CC)[C@@H](O)[C@H]3O)cc2F)cc1]

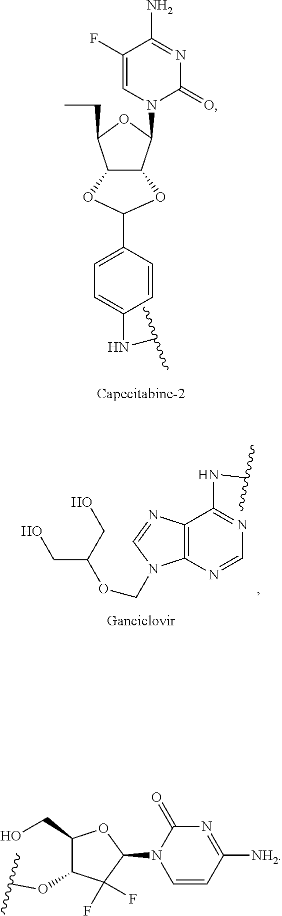

### The Antibody

The antibody (mAb) used for the conjugation process is preferred a cell-binding antibody or antibody-like protein molecule that binds to, complexes with, or reacts with a moiety of a cell population sought to be therapeutically or otherwise biologically modified.

For convenience in this section and elsewhere, “antibody” should be understood to include “antibody-like protein and peptide” except where the context requires otherwise. Suitable antibody-like proteins which may be present in the conjugates of the invention include for example peptides, polypeptides, antibodies, antibody fragments, enzymes, cytokines, chemokines, receptors, blood factors, peptide hormones, toxin, transcription antibody-like proteins, or multimeric antibody-like proteins, wherein they have interchain disulfide bonds structurally.

Enzymes include carbohydrate-specific enzymes, proteolytic enzymes and the like, for example the oxidoreductases, transferases, hydrolases, lyases, isomerases and ligases disclosed by U.S. Pat. No. 4,179,337. Specific enzymes of interest include asparaginase, arginase, adenosine deaminase, superoxide dismutase, catalase, chymotrypsin, lipase, uricase, bilirubin oxidase, glucose oxidase, glucuronidase, galactosidase, glucocerbrosidase, and glutaminase.

Blood antibody-like proteins include albumin, transferrin, Factor VII, Factor VIII or Factor IX, von Willebrand factor, insulin, ACTH, glucagen, somatostatin, somatotropins, thymosin, parathyroid hormone, pigmentary hormones, somatomedins, erythropoietin, luteinizing hormone, hypothalamic releasing factors, antidiuretic hormones, prolactin, interleukins, interferons, for example IFN-α. or IFN—P, colony stimulating factors, haemoglobin, cytokines, antibodies, antibody fragments, chorionicgonadotropin, follicle-stimulating hormone, thyroid stimulating hormone and tissue plasminogen activator.

Other antibody-like proteins of interest are allergen antibody-like proteins disclosed by Dreborg et al Crit. Rev. Therap. Drug Carrier Syst. (1990) 6 315-365 as having reduced allergenicity when conjugated with a polymer such as poly(alkylene oxide) and consequently are suitable for use as tolerance inducers. Among the allergens disclosed are Ragweed antigen E, honeybee venom, mite allergen and the like.

Glycopolypeptides such as immunoglobulins, ovalbumin, lipase, glucocerebrosidase, lectins, tissue plasminogen activator and glycosylated interleukins, interferons and colony stimulating factors are of interest, as are immunoglobulins such as IgG, IgE, IgM, IgA, IgD and fragments thereof. Of particular interest are receptor and ligand binding antibody-like proteins and antibodies and antibody fragments which are used in clinical medicine for diagnostic and therapeutic purposes.

The antibody herein is preferred (A): the group consisting of an antibody, an antibody-like protein molecule, probody, nanobody, peptides, an antibody coating on polymeric micelle, an antibody-liposome, a lipoprotein-based drug carrier, an antibody coating on nano-particle, an antibody-dendrimer, and a particle said above coated or linked with an antibody-like protein (antibody), or a combination of said above thereof;
(B): an antibody, full-length antibodies (polyclonal antibodies, monoclonal antibodies, dimers, multimers, multispecific antibodies (e.g., bispecific antibody, trispecific antibody, or tetraspecific antibody); single chain antibodies; an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune antibody-like proteins (SIP), a lymphokine antibody-like protein, a hormone type antibody-like protein, a growth factor antibody-like protein, a colony stimulating factor antibody-like protein, a nutrient-transport antibody-like protein, large molecular weight antibody-like proteins, fusion antibody-like proteins, a kinase inhibitor antibody-like protein, gene-targeting antibody-like protein, antibody-like protein coated on nanoparticles or polymers modified with antibodies or large molecular weight antibody-like proteins;

The fragments of antibodies include Fab, Fab′, F(ab′)₂, F,, [Parham, J. Immunol. 131, 2895-902 (1983)], fragments produced by a Fab expression library, and epitope-binding fragments of any of the above which immuno-specifically bind to cancer cell antigens, viral antigens, microbial antigens or an antibody-like protein generated by the immune system that is capable of recognizing, binding to a specific antigen or exhibiting the desired biological activity (Miller et al (2003) J. of Immunology 170: 4854-61); interferons (such as type I, II, III); peptides; lymphokines such as IL-2, IL-3, IL-4, IL-5, IL-6, IL-10, GM-CSF, interferon-gamma (IFN-γ); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFa, TGFP, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight antibody-like proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (K5hler, G.; Milstein, C. (1975). Nature 256: 495-7). The detailed procedures are described in “Antibodies--A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0~20 mM glutamine, 0~20% fetal calf serum, several ppm amount of heavy metals, such as Cu, Mn, Fe, or Zn, etc, or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4, 399, 121; 4, 427, 783; 4, 444, 887; 4, 451, 570; 4, 466, 917; 4, 472, 500; 4, 491, 632; 4, 493, 890. A monoclonal antibody may also be produced via an anti-receptor peptide or peptides containing the carboxyl terminal as described well-known in the art. See Niman et al., Proc. Natl. Acad. Sci. USA, 80: 4949-53 (1983); Geysen et al., Proc. Natl. Acad. Sci. USA, 82:178-82 (1985); Lei et al. Biochemistry 34(20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(l):7-13 (1994); Little et al, Biotechnol Adv. 12(3): 539-55 (1994); Clackson et al., Nature 352: 264-8 (1991); Huse et al., Science 246:1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6, 797, 492; Liu et al, Immunol Rev. 222: 9-27 (2008); Almagro et al, Front Biosci. 13:1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6, 207, 418, 6, 150, 584, 6, 111, 166, 6, 075, 181, 5, 922, 545, 5, 661, 016, 5, 545, 806, 5, 436, 149 and 5, 569, 825. In human therapy, murine variable regions and human constant regions can also be fused to construct called “chimeric antibodies” that are considerably less immunogenic in man than murine mAbs (Kipriyanov et al, Mol Biotechnol. 26: 39-60 (2004); Houdebine, Curr Opin Biotechnol. 13: 625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3: 964-70, (2002)); Adams et al, J Immunol Methods. 231: 249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, an antibody like peptide or protein that bind/block/target or in some other way interact with the epitopes or corresponding receptors on a targeted cell can be used as a binding molecule. These antibody-like peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of antibody like peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs of this prevention for treating cancer, autoimmune disease, and/or infectious disease include, but are not limited to, 3F8 (anti-GD₂), Abagovomab (anti CA-125), Abciximab (anti CD41 (integrin alpha-IIb), Adalimumab (anti-TNF-α),

Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti- CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (α chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-P), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CRO11, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (α chain of IL-2 receptor)), Inotuzumab (anti-CD22), Ipilimumab (anti-CD152), Iratumumab (anti- CD30 (TNFRSF8)), Keliximab (anti-CD4), Labetuzumab (CEA-Cide, anti-CEA), Lebrikizumab (anti- IL-13), Lemalesomab (anti-NCA-90 (granulocyte antigen)), Lerdelimumab (anti-TGF beta 2), Lexatumumab (anti-TRAIL-R2), Libivirumab (anti-hepatitis B surface antigen), Lintuzumab (anti-CD33), Lucatumumab (anti-CD40), Lumiliximab (anti- CD23 (IgE receptor), Mapatumumab (anti-TRAIL-R 1), Maslimomab (anti- T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R α), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti- Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), R₁₀uximab (MabThera, R₁₀uxanmab, anti-CD20), Robatumumab (anti-IGF-1 receptor), Rontalizumab (anti-IFN-a), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD₂), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R 2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti- Escherichia coli), Ustekinumab (Stelara, anti-IL-12, IL-23), Vapaliximab (anti-AOC3 (VAP-1)), Vedolizumab, (anti-integrin α₄62 ₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive@), Abatacept (Orencia@), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.28S [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRap (T cell receptor alpha/beta), complex, from MedImmune Inc for Graft-versus-host disease], RING SCAN [anti-TAG 72 (tumor associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C1 1 (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple mycloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple mycloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 or DLL4 (delta-like-3 or delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HERI (lung, stomach cancers), HER² (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A₁ (membrane-spanning 4-domains subfamily A member 1, Non-Hodgkin's B cell lymphoma, leukemia), MUC1 or MUCl-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A₁ (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumor associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R 1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54(3), 187-207 (2005). Franke, et al, Cancer Biother Radiopharm. 2000, 15, 459-76).

The antibody-like protein, more preferred an IgG antibody that is able to against tumor cells, virus infected cells, microorganism infected cells, parasite infected cells, autoimmune disease cells, activated tumor cells, mycloid cells, activated T-cells, an affecting B cells, or melanocytes. More specifically the antibody is able to against abnormal cells expressing any one of the following antigens or receptors: CD1, CD1a, CD1b, CD1c, CD1d, CD1e, CD₂, CD3, CD3d, CD3e, CD3g, CD4, CD5, CD6, CD7, CD8, CD8a, CD8b, CD9, CD10, CD1la, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158bl, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a, b), CD203, CD203c, CD204, CD205, CD206, CD207, CD208, CD209, CD210, CDw210a, CDw210b, CD211, CD212, CD213, CD213al, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, ber-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCLl 1(C—C motif chemokine 11), CCR4 (C—C chemokine receptor type 4), CCR5, CD3E (epsilon), CEA (Carcinoembryonic antigen), CEACAM3, CEACAM5 (carcino-embryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen IF protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Trans-membrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{llb}β3, αvβ, α4β7, α5β1, α6β4, α7β7, αllβ, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD1 1a), LHRH, LINGO-1, Lipoteichoic acid, LIVIA, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), B7H3, B7H4, MSLN (mesothelin), MUC1(Mucin 1, cell surface associated (MUC1) or polymorphic epithelial mucin (PEM)), MUC1-KLH, MUC16 (CA125), MCP1(monocyte chemotactic protein 1), MelanA/MART1, ML-IAP, MPG, MS4A₁ (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY—BR-1, NY-ESO-1, OX-40, OxLDL (Oxidized low-density lipoprotein), OY-TES1, P21, p⁵³ nonmutant, P97, Page4, PAP, Paratope of anti-(N-glycolylneuraminic acid), PAX3, PAX5, PCSK9, PDCD1 (PD-1, Programmed cell death protein 1), PDGF-Ra (Alpha-type platelet-derived growth factor receptor), PDGFR-P, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF1OB (tumor necrosis factor receptor superfamily member 10B), TNFRSF-13B (tumor necrosis factor receptor superfamily member 13B), TPBG (trophoblast glycoprotein), TRAIL-Ri (Tumor necrosis apoptosis Inducing ligand Receptor 1), TRAILR² (Death receptor 5 (DR5)), tumor-associated calcium signal transducer 2, tumor specific glycosylation of MUC1, TWEAK receptor, TYRP1(glycoprotein 75), TRP-1 (Trop1), TRP-2 (Trop2), Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

Many of tumor-associated antigens (TAA) or tumor cell receptors are known in the art, and can be prepared for use in generating antibodies using methods and information which are well known in the art. In attempts to discover effective cellular targets for cancer diagnosis and therapy, researchers have sought to identify transmembrane or otherwise tumor-associated polypeptides or glycoproteins that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via the ADCs of this application. Examples of the TAA and cognate antibodies with their known in art are:
(1) BMPR1B (Bone Morphogenetic Protein Receptor-Type IB);
(2) E16 (LAT1, SLC7A5);
(3) STEAP1 (Six Transmembrane Epithelial Antigen of Prostate),
(4) 0772P (CA125, MUC16);
(5) MPF (MPF, MSLN, SMR, Megakaryocyte Potentiating Factor, Mesothelin);
(6) Napi3b (NAPI-3B, NPTIIb, SLC34A2, Solute Carrier Family 34 (Sodium Phosphate), Member 2, Type II Sodium-Dependent Phosphate Transporter 3b);
(7) Sema 5b (FLJ10372, KIAA1445, Mm.42015, SEMA5B, SEMAG, Semaphorin 5b Hlog, 25 Sema Domain, Seven Thrombospondin Repeats (Type 1 and Type 1-Like), Transmembrane Domain™ and Short Cytoplasmic Domain, (Semaphorin) 5B);
(8) PSCA hlg (2700050C12Rik, C530008016Rik, RIKEN cDNA 2700050C12, RIKEN cDNA 2700050C12 gene);
(9) ETBR (Endothelin Type B Receptor);
(10) MSG783 (RNF124, Hypothetical Protein FLJ20315);
(11) STEAP2 (HGNC_8639, IPCA-1, PCANAP1, STAMP1, STEAP2, STMP, Prostate Cancer Associated Gene 1, Prostate Cancer Associated Protein 1, Six Transmembrane Epithelial Antigen of Prostate 2, Six Transmembrane Prostate Protein);
(12) TrpM4 (BR22450, FLJ20041, TRPM4, TRPM4B, Transient Receptor Potential Cation 5 Channel, Subfamily M, Member 4);
(13) CRIPTO (CR, CR1, CRGF, CRIPTO, TDGF1, Teratocarcinoma-Derived Growth Factor);
(14) CD21 (CR² (Complement Receptor 2) or C3DR (C3d/Epstein Barr Virus Receptor) or Hs. 73792);
(15) CD79b (CD79B, CD793, IGb (Immunoglobulin-Associated Beta), B29);
(16) FcRH2 (IFGP4, IRTA4, SPAP1A (SH2 Domain Containing Phosphatase Anchor Protein 5 La), SPAPIB, SPAPlC);
(17) HER² (ErbB2);
(18) NCA (CEACAM6);
(19) MDP (DPEP1);
(20) IL20R-Alpha (IL20Ra, ZCYTOR7);
(21) Brevican (BCAN, BEHAB);
(22) EphB2R (DRT, ERK, Hek5, EPHT3, Tyro5);
(23) ASLG659 (B7h);
(24) PSCA (Prostate Stem Cell Antigen Precursor);
(25) GEDA;
(26) BAFF-R (B Cell-Activating Factor Receptor, BLyS Receptor 3, BR3);
(27) CD22 (B-Cell Receptor CD22-B Isoform, BL-CAM, Lyb-8, Lyb8, SIGLEC-2, FLJ22814);
(27A) Cd22 (Cd22 Molecule);
(28) CD79a (CD79A, CD79alpha), Immunoglobulin-Associated Alpha, a B Cell-Specific Protein that Covalently Interacts with Ig Beta (CD79B) and Forms a Complex on the Surface with Ig M 35 Molecules, Transduces a Signal Involved in B-Cell Differentiation), P1: 4.84, MW: 25028 Tm: 2 [P] Gene Chromosome: 19q13.2);
(29) CXCR5 (Burkitt's Lymphoma Receptor 1, a G Protein-Coupled Receptor that is Activated by the CXCL13 Chemokine, Functions in Lymphocyte Migration and Humoral Defense, Plays a 10 Role in HIV-2 Infection and Perhaps Development of AIDS, Lymphoma, Myeloma, and Leukemia); 372 Aa, P1: 8.54 MW: 41959 TM: 7 [P] Gene Chromosome: l 1q23.3;
(30) HLA-DOB (Beta Subunit of MHC Class II Molecule (La Antigen) that Binds Peptides and 20 Presents them to CD4+T Lymphocytes); 273 Aa, P1: 6.56, MW: 30820. TM: 1 [P] Gene Chromosome: 6p21.3);
(31) P2X5 (Purinergic Receptor P2X Ligand-Gated Ion Channel 5, an Ion Channel Gated by Extracellular ATP, May be Involved in Synaptic Transmission and Neurogenesis, Deficiency May Contribute to the Pathophysiology of Idiopathic Detrusor Instability); 422 Aa), P1: 7.63, MW: 47206 TM: 1 [P] Gene Chromosome: 17pl3.3);
(32) CD72 (B-Cell Differentiation Antigen CD72, Lyb-2); 359 Aa, P1: 8.66, MW: 40225, TM: 1 5 [P] Gene Chromosome: 9pl3.3);
(33) LY64 (Lymphocyte Antigen 64 (RP105), Type I Membrane Protein of the Leucine Rich Repeat (LRR) Family, Regulates B-Cell Activation and Apoptosis, Loss of Function is Associated with Increased Disease Activity in Patients with Systemic Lupus Erythematosis); 661 Aa, pl: 6.20, MW: 74147 TM: 1 [P] Gene Chromosome: 5ql2);
(34) FcRH1 (Fc Receptor-Like Protein 1, a Putative Receptor for the Immunoglobulin Fc Domain that Contains C2 Type Ig-Like and ITAM Domains, may have a Role in B-Lymphocyte 20 Differentiation); 429 Aa, P1: 5.28, MW: 46925 TM: 1 [P] Gene Chromosome: 1q21-1q22);
(35) IRTA2 (Immunoglobulin Superfamily Receptor Translocation Associated 2, a Putative Immunoreceptor with Possible Roles in B Cell Development and Lymphomagenesis; Deregulation of the Gene by Translocation Occurs in Some B Cell Malignancies); 977 Aa, pl: 6.88, MW: 106468, TM: 1 [P] Gene Chromosome: 1q21);
(36) TENB2 (TMEFF2, Tomoregulin, TPEF, HPP1, TR, Putative Transmembrane 35 Proteoglycan, Related to the EGF/Heregulin Family of Growth Factors and Follistatin); 374 Aa);
(37) PSMA-FOLH1 (Folate Hydrolase (Prostate-Specific Membrane Antigen) 1);
(38) SST (Somatostatin Receptor; Note that there are 5 Subtypes);
(38.1) SSTR² (Somatostatin Receptor 2);
(38.2) SSTR5 (Somatostatin Receptor 5);
(38.3) SSTR1; (38.4) SSTR3; (38.5) SSTR4; AvB6-Both Subunits (39+40);
(39) ITGAV (Integrin, Alpha V);
(40) ITGB6 (Integrin, Beta 6);
(41) CEACAM5 (Carcinoembryonic Antigen-Related Cell Adhesion Molecule 5);
(42) MET (Met Proto-Oncogene; Hepatocyte Growth Factor Receptor);
(43) MUC1 (Mucin 1, Cell Surface Associated);
(44) CA9 (Carbonic Anhydrase IX);
(45) EGFRvIII (Epidermal Growth Factor Receptor (EGFR), Transcript Variant 3);
(46) CD33 (Cd33 Molecule);
(47) CD19 (Cdl9 Molecule):
(48) IL₂RA (Interleukin 2 Receptor, Alpha); NCBI Reference Sequence: NM_000417.2);
(49) AXL (AXL Receptor Tyrosine Kinase);
(50) CD30 TNFRSF8 (Tumor Necrosis Factor Receptor Superfamily, Member 8);
(51) BCMA (B-Cell Maturation Antigen) TNFRSF17 (Tumor Necrosis Factor Receptor Superfamily, Member 17);
(52) CT Ags CTA (Cancer Testis Antigens);
(53) CD174 (Lewis Y)-FUT3 (Fucosyltransferase 3 (Galactoside 3(4)-L-Fucosyltransferase, Lewis Blood Group);
(54) CLECl4A (C-Type Lectin Domain Family 14, Member a;
(55) GRP78 HSPA5 (Heat Shock 70 kDa Protein 5 (Glucose-Regulated Protein, 78 kDa);
(56) CD70 (Cd70 Molecule) L08096;
(57) Stem Cell Specific Antigens: For Example: 5T4 (see entry (63) below); CD25 (see entry
(48) above);
(58) ASG-5;
(59) ENPP3 (Ectonucleotide Pyrophosphatase/Phosphodiesterase 3);
(60) PRR4 (Proline Rich 4 (Lacrimal));
(61) GCC GUCY2C (Guanylate Cyclase 2C (Heat Stable Enterotoxin Receptor);
(62) Liv-1-SLC39A6 (Solute Carrier Family 39 (Zinc Transporter), Member 6);
(63) 5T4, Trophoblast Glycoprotein, TPBG TPBG (Trophoblast Glycoprotein);
(64) CD56 NCMA 1 (Neural Cell Adhesion Molecule 1);
(65) CanAg (Tumor Associated Antigen CA242);
(66) FOLR1 (Folate Receptor 1);
(67) GPNMB (Glycoprotein (Transmembrane) Nmb);
(68) TIM-1 HAVCRI (Hepatitis a Virus Cellular Receptor 1);
(69) RG-1/Prostate Tumor Target Mindin-Mindin/RG-1;
(70) B7-H4-VTCN1 (V-Set Domain Containing T Cell Activation Inhibitor 1;
(71) PTK7 (PTK7 Protein Tyrosine Kinase 7);
(72) CD37 (Cd37 Molecule);
(73) CD138 SDCI (Syndecan 1);
(74) CD74 (CD74 Molecule, Major Histocompatibility Complex, Class II Invariant Chain);
(75) Claudins-CLs (Claudins);
(76) EGFR (Epidermal Growth Factor Receptor);
(77) Her3 (ErbB3)-ERBB3 (v-Erb-b2 Erythroblastic Leukemia Viral Oncogene Homolog 3
(Avian));
(78) RON MST1R (Macrophage Stimulating 1 Receptor (c-Met-Related Tyrosine Kinase));
(79) EPHA2 (EPH Receptor A2);
(80) CD20 MS4A₁ (Membrane-Spanning 4-Domains, Subfamily a, Member 1);
(81) Tenascin C TNC (Tenascin C);
(82) FAP (Fibroblast Activation Protein, Alpha);
(83) DKK-1 (Dickkopf 1 Homolog (Xenopus laevis);
(84) CD52 (Cd52 Molecule);
(85) CS -SLAMF7 (SLAM Family Member 7);
(86) Endoglin ENG (Endoglin);
(87) PMEL17 (silver homolog; SILV; D12S53E; PMEL17; SI; SIL); ME20; gp100) BC001414; BT007202; M32295; M77348; NM_006928;
(88) TMEFF1 (transmembrane protein with EGF-like and two follistatin-like domains 1; Tomoregulin-1); H7365; C9orf2; C90RF2; U19878; X83961; NM_080655; NM_003692;
(89) GDNF-Ral (GDNF family receptor alpha 1; GFRA1; GDNFR; GDNFRA; RETLi; TRNR₁; RET1L; GDNFR-alphal; GFR-ALPHA-1); U95847; BC014962; NM 145793 NM_005264;
(90) Ly6E (lymphocyte antigen 6 complex, locus E; Ly67, RIG-E, SCA-2, TSA-1); NP_002337.1; NM_002346.2;
(91) TMEM46 (shisa homolog 2 (Xenopus laevis); SHISA2); NP_001007539.1; NM_001007538.1;
(92) Ly6G6D (lymphocyte antigen 6 complex, locus G6D; Ly6-D, MEGT1); NP_067079.2; NM_021246.2;
(93) LGR5 (leucine-rich repeat-containing G protein-coupled receptor 5; GPR49, GPR67); NP_003658.1; NM_003667.2;
(94) RET (ret proto-oncogene; MEN2A; HSCR1; MEN2B; MTC1; PTC; CDHF12; Hs.168114; RET51; RET-ELEl); NP_066124.1; NM_020975.4;
(95) LY6K (lymphocyte antigen 6 complex, locus K; LY6K; HSJ001348; FLJ35226); NP_059997.3; NM_017527.3;
(96) GPR19 (G protein-coupled receptor 19; Mm.4787); NP_006134.1; NM_006143.2;
(97) GPR54 (KISS1 receptor; KISSIR; GPR54; HOT7T175; AXOR12); NP_115940.2; NM_032551.4;
(98) ASPHDI (aspartate beta-hydroxylase domain containing 1; LOC253982); NP_859069.2; NM_181718.3;
(99) Tyrosinase (TYR; OCAIA; OCAlA; tyrosinase; SHEP3); NP_000363.1; NM_000372.4;
(100) TMEM118 (ring finger protein, transmembrane 2; RNFT2; FLJ14627); NP_001103373.1; NM_001109903.1;
(101) GPR172A (G protein-coupled receptor 172A; GPCR41; FLJ11856; D15Ertd747e); NP_078807.1; NM_024531.3;
(102) CLL-1 (CLECl2A, MICL, and DCAL₂), encodes a member of the C-type lectin/C-type lectin-like domain (CTL/CTLD) superfamily;
(103) Annexin A1-ANXA1 (Annexin A1);
(104) V-CAM (CD106) VCAM1 (Vascular Cell Adhesion Molecule 1);
(105) B7H3 (CD276 or B7RP-2, a member of the B7 ligand family, has two isoforms: 21g- and 41g-B7H3 with molecular weights of approximately 45-kDa and 100-kDa, respectively);
(106) CD54 (also Known as BB2; CD54; P3.58);
(107) CA19-9 (Carbohydrate antigen 19-9 (CA 19-9) is a cell surface glycoprotein complex most commonly associated with pancreatic ductal adenocarcinoma (PDAC);
(108) Tissue Factor (coagulation factor III, tissue factor, TF; TFA; CD142);
(109) ROR1 (receptor tyrosine kinase like orphan receptor 1, NTRKR1; dJ537F10.1);
(110) Claudin18 (SFTA5; SFTPJ);
(111) FGFR2(fibroblast growth factor receptor 2, BEK; JWS; BBDS; CEK3; CFD1; ECT1; KGFR; TK14; TK25; BFR-1; CD332; K-SAM);
(112) FGFR3 (fibroblast growth factor receptor 3, ACH; CEK2; JTK4; CD333; HSFGFR3EX);
(113) FGFR4 (fibroblast growth factor receptor 4, TKF; JTK2; CD334);
(114) FGFR1 (fibroblast growth factor receptor 1, CEK; FLG; HH2; OGD; ECCL; FLT2; KAL₂; BFGFR; CD331; FGFBR; FLT-2; HBGFR; N-SAM; FGFR-1; HRTFDS; bFGF-R-1);
(115) ROR² (receptor tyrosine kinase like orphan receptor 2, BDB; BDB1; NTRKR2);
(116) SLC44A4 (SLC44A4-solute carrier family 44 member 4, CTL4; NG22; TPPT; DFNA72; hTPPTI; C₆orf29);
(117) DLL3 (delta like canonical Notch ligand 3, SCDO1);
(118) DLL4 (delta like canonical Notch ligand 4, AOS6; delta4; hdelta2);
(119) ALK (ALK receptor tyrosine kinase, ALKI; CD246; NBLST3);
(120) CLDN6 (claudin 6);
(121) CLDN3 (claudin 3, RVPl; HRVP1; C7orfl; CPE-R2; CPETR2);
(122) EFNA4 (ephrin A4, EFL4; EPLG4; LERK4; LERK-4);
(123) Notchl(notch receptor 1, hNl; AOS5; TANI; AOVD1)
(124) Notch2 (notch receptor 2, hN2; AGS2; HJCYS);
(125) Notch3 (notch receptor 3, IMF2; LMNS; CASIL; CADASIL; CADASIL1);
(126) LAMP-1 (lysosomal associated membrane protein 1, LAMPA; CD107a; LGP120); Many of the above antigens were described in our previous patent application (PCT/CN2023/096066 field on May 24^{th}, 2023).

In another specific embodiment, the antibody-drug conjugates of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the Myeloproliferative antibody-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barra syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, M6niere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sj5gren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome In another specific embodiment, the antibody-drug conjugates of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.lRNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial(M2) antibody; Rheumatoid factor antibody; Anti-MCV antibody; Anti-topoisomerase antibody; Anti-neutrophil cytoplasmic(cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD₂, CD3, CD4, CD8, CD19, CD20, CD22, CD28, CD30, CD33, CD37, CD38, CD56, CD70, CD79, CD79b, CD90, CD125, CD137, CD138, CD147, CD152/CTLA-4, PD-1, or ICOS), a TNF receptor superfamily member (e.g. CD27, CD40, CD95/Fas, CD134/OX40, CD137/4-1BB, INF—R1, TNFR-2, RANK, TACI, BCMA, osteoprotegerin, Apo2/TRAIL-R1, TRAIL-R2, TRAIL-R₃, TRAIL-R4, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimidase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., bacteria, fungi, pathogenic protozoa, or yeast polypeptides including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PR0542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The antibody-drug conjugates of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Striussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The cell binding molecule, which is more preferred to be an antibody described in this patent that are against pathogenic strains include, but are not limit, Acinetobacter baumannii, Actinomyces israelii, Actinomyces gerencseriae and Propionibacterium propionicus, Trypanosoma brucci, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4) - Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157:H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprac and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as cell binding ligands used in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

In further preferred embodiments, the present invention also provides an antibody-drug conjugate (ADC) comprising a monoclonal antibody, or an antigen-binding fragment thereof, conjugated with a cytotoxin, via a linker containing a glutamate urea small molecule, such as 2-[3-(1,3-dicarboxypropyl)ureido]-pentanedioic acid (DUPA), urea-based glutamate heterodimers, 2-(phosphonomethyl)-pentanedioic acid (PMPA), phosphoramidates, glu-urea-lys, or 2-(phosphinylmethyl) pentanedioic acids analog group to direct against prostate antigen (PSA) of a tumor cell, and/or an affinity ligand for bombesin receptors (Gastrin releasing peptide receptor (GRPR), neurotensin receptors (including Neurotensin receptor 1 (NTR1) and neuropeptide-Y receptors), and/or a cell-penetrating peptide, and/or an affinity peptide that can bind with a protein called programmed death ligand-1 (PD-L1, or CD274) which is expressed on tumor cells and tumor-infiltrating immune cells, blocking its interactions with both PD-1 and B7.1 receptors. The affinity to the receptors is at least EC₅₀<10 μM, preferably EC₅₀<100 nM, and more preferably EC₅₀<50 nM. In a further embodiment the antigen binding proteins are conjugated to a cytotoxin, such as, but not limited, a tubulysin analog, a camptothecin (CPT) analog, a PBD dimer, an anthracycline, or an auristatin analog.

In some embodiments, the cell-penetrating peptide (CPP) used in this invention can be selected from CPP database (http://crdd.osdd.net/raghava/cppsite) or from known publications with less than 100 amino acids of sequences or from amendment of known peptide sequences with replacement of one or several amino acids, and then is subjected to redundancy check. The preferred CPP is a linear or cyclo- peptide having less than 50 amino acids, preferably less than 20 natural or unnatural amino acids, more preferably less than 15 amino acids and containing one, two, or several arginines and/or lysines. The CPP is more preferably a cyclopeptide, in particular CPP is a cyclopeptide having less than 8 amino acids. The selected peptides are normally further analyzed to filter out the ambiguous peptides with undesirable chemical modifications. The amphipathicity prediction can be through the online server AMPHIPASEEK (https:// npsa-prabi.ibcp.fr/cgibin/npsa_automat.pl?page=/NPSA/npsa_amphipaseek.html). AMPHIPASEEK provides a score for every residue between a range of 0 and 5 for the given peptide sequences. Higher scores imply high amphipathic nature and vice versa (0=low and 5=high). Hydropathy values were (K-Me3) calculated using the online server (https://www.peptide2.com/N_peptide_hydrophobicity_hydrophilicity.php). The CPPs are normally required to pass through the criteria of peptide solubility and the cell-penetrating property using Innovagen peptide solubility calculator (https://pepcalc.com/) and CPPpred (http://bioware.ucd.ie/-compass/biowareweb/Server_pages/cpppred.php), respectively. The CPP score is given within the range of 0-1, wherein the peptides with the score of>0.5 are suggestive of better cell penetration. The efficiency of CPP penetration of a cell can be measured in several different methods (Lee, H-M, et al, Nature Communications Biology 2021, 4:205; Penedo, M. et al, Scientific Reports, 2021, 11:7756 and the references they incorporated). In general, the preferable CPP should enable to internalize (trafficking) over 40% of the ligand bound on a cell or help to internalize 40% of ADCs bound on a cell to cross the cell membrane in 2 hours.

In some embodiments, the present invention provides antigen binding antibody-drug conjugates which bind to membrane bound targets and wherein the antigen binding ADC is capable of internalization. In a further embodiment there is provided an immunoconjugate comprising the antigen binding protein of the present invention and a cytotoxic agent. In a further embodiment the antigen binding protein has ADCC effector function for example the antigen binding protein has enhanced ADCC effector function. In one such embodiment there is provided antigen binding antibodies/proteins or fragments of the antibodies used for ADCs against various cancers thereof.

In one aspect of the invention, the provided an antibody/protein used for the antibody-drug conjugate of this invention is preferably selected from an antibody having affinity to an antigen of highly expressed on a tumor cell The information including the sequences of the provided antibody can be found in the known public domains, such as in the databases of patents in WIPO, USPTO, Espacenet, CNIPA, JPO, etc.

The antigen binding antibodies/proteins of the present invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full-length antibody, a (Fab′)2 fragment, a Fab fragment, or equivalent thereof (such as scFV, bi- tri- or tetra-bodies, Tandabs etc.), when paired with an appropriate light chain. The antibody may be an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. Furthermore, the antigen binding protein may comprise modifications of all classes e.g. IgG dimers, Fc mutants that no longer bind Fc receptors or mediate C1q binding. The antigen binding protein may also be a chimeric antibody of the type described in WO86/001533 which comprises an antigen binding region and a non-immunoglobulin region.

The constant region is selected according to any functionality required e.g. an IgG1 may demonstrate lytic ability through binding to complement and/or will mediate ADCC (antibody dependent cell cytotoxicity).

In one aspect the antigen binding protein is an antibody or antigen binding fragment thereof comprising one or more CDR's according to the invention described herein, or one or both of the heavy or light chain variable domains according to the invention described herein. The antigen binding protein is selected from the group consisting of a dAb, Fab, Fab′, F(ab′)₂, Fv, diabody, triabody, tetrabody, miniantibody, and a minibody.

In one aspect of the present invention the antigen binding protein is a humanised or chimaeric antibody, in a further aspect the antibody is humanised. In one aspect the antibody is a monoclonal antibody.

In another aspect the antigen binding protein binds to human antigens with high affinity for example when measured by Biacore or ForteBio, the antigen binding protein binds to human antigens with an affinity of 20 nM or less or an affinity of 15 nM or less or an affinity of 5 nM or less or an affinity of 1000 μM or less or an affinity of 500 μM or less or an affinity of 400 μM or less, or 300 μM or less or for example about 120 μM. In a further embodiment the antigen binding protein binds to human antigens when measured by Biacore of between about 100 μM and about 500 μM or between about 100 μM and about 400 μM, or between about 100 μM and about 300 μM. In one embodiment of the present invention the antigen binding protein binds antigens with an affinity of less than 150 μM.

In one such embodiment, this is measured by Biacore or ForteBio.

In another aspect the antigen binding protein/antibody binds to human antigens in a cell neutralisation assay wherein the antigen binding protein has an IC₅₀ of between about 1 nM and about 500 nM, or between about 1 nM and about 100 nM, or between about 1 nM and about 50 nM, or between about 1 nM and about 25 nM, or between about 5 nM and about 15 nM. In a further embodiment of the present invention the antigen binding protein binds antigens and neutralises antigens in a cell neutralisation assay wherein the antigen binding protein has an IC₅₀of about 10 nM.

The antigen binding proteins, for example antibodies of the present invention may be produced by transfection of a host cell with an expression vector comprising the coding sequence for the antigen binding protein of the invention. An expression vector or recombinant plasmid is produced by placing these coding sequences for the antigen binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen binding protein light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen binding protein may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen binding protein of the invention. The antigen binding protein which includes the association of both the recombinant heavy chain and/or light chain is screened from culture by appropriate assay, such as ELISA or RIA. Similar conventional techniques may be employed to construct other antigen binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham Bioscience (Buckinghamshire, United Kingdom) or GenScript (Nanjing, China). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen binding proteins of the present invention. Host cells useful for the cloning and other manipulations of these cloning vectors are also conventional. However, cells from various strains of E. Coli may be used for replication of the cloning vectors and other steps in the construction of antigen binding proteins of this invention.

Suitable host cells or cell lines for the expression of the antigen binding proteins of the invention include mammalian cells such as NSO, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns.

Alternatively, other eukaryotic cell lines may be employed. The selection of suitable mammalian host cells and methods for transformation, culture, amplification, screening and product production and purification are known in the art. See, e.g., Sambrook et al., (1989). Molecular cloning: a laboratory manual, 2nd ed. Cold Spring Harbor Laboratory, Cold Spring Harbor, N.Y.

Bacterial cells may prove useful as host cells suitable for the expression of the recombinant Fabs or other embodiments of the present invention (see, e.g., Pluckthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. Coli used for expression are well-known as host cells in the field of biotechnology. Various strains of B. Subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and McGuire, S. et al, Trends Genet. (2004) 20, 384-391 and references cited therein.

The general methods by which the vectors may be constructed, the transfection methods required to produce the host cells of the invention, and culture methods necessary to produce the antigen binding protein of the invention from such host cell may all be conventional techniques.

Typically, the culture method of the present invention is a serum-free culture method, usually by culturing cells serum-free in suspension. Likewise, once produced, the antigen binding proteins of the invention may be purified from the cell culture contents according to standard procedures of the art, including ammonium precipitation, affinity columns, column chromatography, gel electrophoresis and the like. Such techniques are within the skill of the art and do not limit this invention. For example, preparations of altered antibodies are described in WO 99/058679 and WO 96/016990. Yet another method of expression of the antigen binding proteins may utilize expression in a transgenic animal, such as described in U.S. Pat. No. 4,873,316. This relates to an expression system using the animals casein promoter which when transgenically incorporated into a mammal permits the female to produce the desired recombinant protein in its milk.

In a further embodiment of the invention there is provided a method of producing an antibody of the invention which method comprises the step of culturing a host cell transformed or transfected with a vector encoding the light and/or heavy chain of the antibody of the invention and recovering the antibody thereby produced.

In accordance with the present invention there is provided a method of producing an antibody of the present invention which binds to and neutralises the activity of human ANTIGENS which method comprises the steps of; providing a first vector encoding a heavy chain of the antibody; providing a second vector encoding a light chain of the antibody; transforming a mammalian host cell (e.g. CHO) with said first and second vectors; culturing the host cell of step (c) under conditions conducive to the secretion of the antibody from said host cell into said culture media; recovering the secreted antibody of step (d).

Once expressed by the desired method, the antibody is then examined for in vitro activity by use of an appropriate assay. Presently conventional ELISA assay formats are employed to assess qualitative and quantitative binding of the antibody to ANTIGENS. Additionally, other in vitro assays may also be used to verify neutralizing efficacy prior to subsequent human clinical studies performed to evaluate the persistence of the antibody in the body despite the usual clearance mechanisms.

The dose and duration of treatment relates to the relative duration of the molecules (the antibody and the antibody-drug conjugate) of the present invention in the human circulation, and can be adjusted by one of skill in the art depending upon the condition being treated and the general health of the patient. It is envisaged that repeated dosing (e.g. once a week or once every two weeks or once every 3 weeks or once every 4 weeks) over an extended time period (e.g. four to six months) may be required to achieve maximal therapeutic efficacy.

In one embodiment of the present invention there is provided a recombinant transformed, transfected or transduced host cell comprising at least one expression cassette, for example where the expression cassette comprises a polynucleotide encoding a heavy chain of an antigen binding protein according to the invention described herein and further comprises a polynucleotide encoding a light chain of an antigen binding protein according to the invention described herein or where there are two expression cassettes and the 1.sup.st encodes the light chain and the second encodes the heavy chain.

For example in one embodiment the first expression cassette comprises a polynucleotide encoding a heavy chain of an antigen binding protein comprising a constant region or antigen binding fragment thereof which is linked to a constant region according to the invention described herein and further comprises a second cassette comprising a polynucleotide encoding a light chain of an antigen binding protein comprising a constant region or antigen binding fragment thereof which is linked to a constant region according to the invention described herein for example the first expression cassette comprises a polynucleotide encoding a heavy chain and a second expression cassette comprising a polynucleotide encoding a light chain.

In another embodiment of the invention there is provided a stably transformed host cell comprising a vector comprising one or more expression cassettes encoding a heavy chain and/or a light chain of the antibody comprising a constant region or antigen binding fragment thereof which is linked to a constant region as described herein. For example, such host cells may comprise a first vector encoding the light chain and a second vector encoding the heavy chain, for example the first vector encodes a heavy chain and a second vector encoding a light chain.

In another embodiment of the present invention there is provided a host cell according to the invention described herein wherein the cell is eukaryotic, for example where the cell is mammalian. Examples of such cell lines include CHO or NSO.

In another embodiment of the present invention there is provided a method for the production of an antibody comprising a constant region or antigen binding fragment thereof which is linked to a constant region according to the invention described herein which method comprises the step of culturing a host cell in a culture media, for example serum-free culture media.

In another embodiment of the present invention there is provided a method according to the invention described herein wherein said antibody is further purified to at least 95% or greater (e.g. 98% or greater) with respect to said antibody containing serum-free culture media. In yet another embodiment there is provided a pharmaceutical composition comprising an antigen binding protein and a pharmaceutically acceptable carrier.

In another embodiment of the present invention there is provided a kit-of-parts comprising the composition according to the invention described herein described together with instructions for use.

The mode of administration of the therapeutic agent of the invention may be any suitable route which delivers the agent to the host. The antigen binding proteins, and pharmaceutical compositions of the invention are particularly useful for parenteral administration, i.e., subcutaneously (s.c.), intrathecally, intraperitoneally, intramuscularly (i.m.) or intravenously (i.v.). In one such embodiment the antigen binding proteins of the present invention are administered intravenously or subcutaneously.

Therapeutic agents of the invention may be prepared as pharmaceutical compositions containing an effective amount of the antigen binding protein of the invention as an active ingredient in a pharmaceutically acceptable carrier. In one embodiment the prophylactic agent of the invention is an aqueous suspension or solution containing the antigen binding protein in a form ready for injection. In one embodiment the suspension or solution is buffered at physiological pH. In one embodiment the compositions for parenteral administration will comprise a solution of the antigen binding protein of the invention or a cocktail thereof dissolved in a pharmaceutically acceptable carrier. In one embodiment the carrier is an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as about 15 or 20% by weight and will be selected primarily based on fluid volumes, viscosities, etc., according to the particular mode of administration selected.

Thus, a pharmaceutical composition of the invention for intravenous infusion could be made up to contain about 250 ml of sterile Ringer's solution, and about 1 to about 30 or 5 mg to about 25 mg of an antigen binding protein of the invention per ml of Ringer's solution. Actual methods for preparing parenterally administrable compositions are well known or will be apparent to those skilled in the art and are described in more detail in, for example, Remington's Pharmaceutical Science, 15.sup.th ed., Mack Publishing Company, Easton, PA, USA. For the preparation of intravenously administrable antigen binding protein formulations of the invention see Parkins D. and Lasmar U. “The formulation of Biopharmaceutical products”, Pharm. Sci. Tech. Today, 3 (2000) 129-137; Wang, W “Instability, stabilisation and formulation of liquid protein pharmaceuticals”, Int. J. Pharm 185 (1999) 129-188; Jorgensen, L. et al, “Recent trends in stabilizing peptides and proteins in pharmaceutical formulation -considerations in the choice of excipients” Expert Opin Drug Deliv. 6 (2009) 1219-1230; Akers, M. J. “Excipient-Drug interactions in Parenteral Formulations”, J. Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J. Pharm. Pharmacol, 54 (2002) 1033-1039; Johnson, R, et al “Mannitol-sucrose mixtures--versatile formulations for protein lyophilization”, J. Pharm. Sci, 91 (2002) 914-922; Kerwin B. “Polysorbates 20 and 80 used in the formulation of protein biotherapeutics: structure and degradation pathways” J. Pharm Sci. 97 (2008) 2924-2935; Ha, E., et al “Peroxide formation in polysorbate 80 and protein stability”, J. Pharm Sci, 91 (2002), 2252-2264, and He, F., et al, “Effect of sugar molecules on the viscosity of high concentration monoclonal antibody solutions” Pharm Res. 28 (2011) 1552-1560; and the entire contents of which are incorporated herein by reference and to which the reader is specifically referred.

In one embodiment the antibody of the invention, when in a pharmaceutical preparation, is present in unit dose forms. The appropriate therapeutically effective dose will be determined readily by those of skill in the art. Suitable doses may be calculated for patients according to their weight, for example suitable doses may be in the range of about 0.1 to about 200 mg/kg, for example about 1 to about 20 mg/kg, for example about 10 to about 20 mg/kg or for example about 1 to about 15 mg/kg, for example about 5 to about 15 mg/kg. To effectively treat conditions such as Multiple myeloma, SLE or IPT in a human, suitable doses may be within the range of about 0.1 to about 2000 mg, for example about 0.1 to about 500 mg, for example about 500 mg, for example about 0.1 to about 150 mg, or about 0.1 to about 80 mg, or about 0.1 to about 60 mg, or about 0.1 to about 40 mg, or for example about 1 to about 100 mg, or about 1 to about 50 mg, of an antigen binding protein of this invention, which may be administered parenterally, for example subcutaneously, intravenously or intramuscularly. Such dose may, if necessary, be repeated at appropriate time intervals selected as appropriate by a physician.

The antigen binding proteins described herein can be lyophilized for storage and reconstituted in a suitable carrier prior to use. This technique has been shown to be effective with conventional immunoglobulins and art-known peroxidise and reconstitution techniques can be employed.

In another aspect of the invention there is provided an antigen binding protein as herein described for use in a medicament.

In one aspect of the present invention there is provided an antigen binding protein according to the invention as herein described for use in the treatment of rheumatoid arthritis, Type 1 Diabetes Mellitus, multiple sclerosis or psoriasis wherein said method comprises the step of administering to said patient a therapeutically effective amount of the antigen binding protein as described herein.

In one embodiment of the present invention, methods are provided for treating cancer in a human comprising administering to said human an antigen binding protein that specifically binds to antigens on the tumor cells. In some instances, the antigen binding protein is part of an immunoconjugate.

The term “antibody-drug conjugate (ADC),” as used herein, refers to a compound comprising a monoclonal antibody (mAb) attached to a cytotoxic agent (generally a small molecule drug with a high systemic toxicity) via chemical linkers. The ADC of this invention is represented as the formula of:

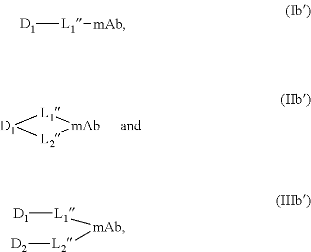

wherein D₁ and D₂ are a small molecule cytotoxin or a functional small molecule, in general called payload; L₁ and L₂ are a function linker that has an affinity ligand; and mAb is a monoclonal antibody. In some embodiments, an ADC may comprise a small molecule cytotoxin that has been chemically modified to contain a linker with an affinity ligand, or a linker containing an affinity ligand is part of payload which is called a traceless linker. The linker is generally used to conjugate the cytotoxin to the antibody, or antigen-binding fragment thereof. Upon binding to the target antigen on the surface of a cell, the ADC is internalized and trafficked to the lysosome where the cytotoxin is released by either proteolysis of a cleavable linker (e.g., by cathepsin B found in the lysosome) or by proteolytic degradation of the antibody, if attached to the cytotoxin via a non-cleavable linker. The cytotoxin then translocates out of the lysosome and into the cytosol or nucleus, where it can then bind to its target, depending on its mechanism of action.

The antibody-drug conjugate described herein may comprise a whole antibody or an antibody fragment. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHI, CH2 and CH3) regions, and each light chain contains one N-terminal variable (VL) region and one C-terminal constant (CL) region. The variable regions of each pair of light and heavy chains form the antigen binding site of an antibody. The VH and VL regions have the same general structure, with each region comprising four framework regions, whose sequences are relatively conserved. The framework regions are connected by three complementarity determining regions (CDR₈). The three CDRs, known as CDR1, CDR2, and CDR3, form the “hypervariable region” of an antibody, which is responsible for antigen binding.

The ADC may comprise an antigen-binding fragment of an antibody. The terms “antibody fragment,” “antigen-binding fragment,” “functional fragment of an antibody,” and “antigen-binding portion” are used interchangeably herein and refer to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Kabat EA, Wu TT., J Immunol. 1991, 147(5): 1709-19) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites (see, e.g. Hudson PJ, Kortt AA, J Immunol Methods. 1999, 231(1-2): 177-89; Holliger P, Winter G. Cancer Immunol Immunother. 1997, 45(3-4):128-30).

The monoclonal antibody, or an antigen-binding fragment thereof, directed against a certain antigen may comprise any suitable binding affinity to the antigen or an epitope thereof. The term “affinity” refers to the equilibrium constant for the reversible binding of two agents and is expressed as the dissociation constant (K_{D}). The affinity of an antibody or antigen-binding fragment thereof for an antigen or epitope of interest can be measured using any method known in the art. Such methods include, for example, fluorescence activated cell sorting (FACS), surface plasmon resonance (e.g., Biacore™, ProteOn™), biolayer interferometry (BLI, e.g. Octet), kinetics exclusion assay (e.g. KinExA™), separable beads (e.g., magnetic beads), antigen panning, and/or ELISA (see, e.g., J R Crowther, Methods Mol Biol. 2000, 149: III-IV, 1-413). It is known in the art that the binding affinity of a particular antibody will vary depending on the method that is used to analyze the binding affinity.

Affinity of a binding agent to a ligand, such as affinity of an antibody for an epitope, can be, for example, from about 1 picomolar (pM) to about 1 micromolar (1 μM) (e.g., from about 1 picomolar (pM) to about 1 nanomolar (nM), or from about 1 nM to about 1 micromolar (pM)). In one embodiment, the monoclonal antibody or an antigen-binding fragment thereof may bind to a certain antigen with a Kd less than or equal to 100 nanomolar (e.g., 100 nM, about 90 nM, about 80 nM, about 70 nM, about 60 nM, about 50 nM, about 40 nM, about 30 nM, about 20 nM, or about 10 nM, or a range defined by any two of the foregoing values).

In another embodiment, the monoclonal antibody may bind to a certain antigen with a Kd less than or equal to 10 nanomolar (e.g., about 9 nM, about 8 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.05 nM, about 0.02 nM, about 0.01 nM, about 0.001 nM, or a range defined by any two of the foregoing values).

In another embodiment, the monoclonal antibody may bind to A CERTAIN ANTIGEN with a Kd less than or equal to 200 μM (e.g., about 190 μM, about 175 μM, about 150 μM, about 125 μM, about 110 μM, about 100 μM, about 90 μM, about 80 μM, about 70 μM, about 60 μM, about 50 μM, about 40 μM, about 30 μM, about 25 μM, about 20 μM, about 15 μM, about 10 μM, about 5 μM, about 1 μM, or a range defined by any two of the foregoing values).

In one embodiment, the affinity of the antibody or antigen-binding fragment thereof, as measured by surface plasmon resonance (SPR), is about 90 nM, about 80 nM, about 70 nM, about 60 nM, about 50 nM, about 40 nM, about 30 nM, or a range defined by any two of the foregoing values, for example, about 50 nM to about 70 nM, about 55 nM to about 65 nM, or about 58 nM to about 62 nM.

In one embodiment, the affinity of the antibody or antigen-binding fragment thereof to membrane-bound antigens, as measured by FACS, is less than or equal to 10 nanomolar (e.g., about 9 nM, about 8 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.05 nM, about 0.02 nM, about 0.01 nM, about 0.001 nM, or a range defined by any two of the foregoing values).

An antigen-binding portion or fragment of a monoclonal antibody can be of any size so long as the portion binds to the antigens. In this respect, an antigen binding portion or fragment of the monoclonal antibody directed against a certain antigen desirably comprises between about 5 and 50 amino acids (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or a range defined by any two of the foregoing values).

In one embodiment, the antibody-drug conjugate comprises a variable region of a monoclonal antibody. In this respect, the ADC may comprise a light chain variable region, a heavy chain variable region, or both a light chain variable region and a heavy chain variable region of a monoclonal antibody.

The monoclonal antibody, or antigen-binding fragment thereof, may be conjugated to a cytotoxin using any suitable method known in the art, including site-specific or non-site-specific conjugation methods. Conventional conjugation strategies for antibodies typically rely on randomly (i.e., non-specifically) conjugating the payload to the antibody, antigen-binding fragment thereof, through lysines or cysteines. Accordingly, in some aspects the antibody or antigen-binding fragment thereof is randomly conjugated to a cytotoxic agent, for example, by partial reduction of the antibody or antibody fragment, followed by reaction with a desired agent with or without a linker moiety attached.

For example, the antibody or antigen-binding fragment thereof may be reduced using dithiothreitol (DTT), TCEP, thioethanol or a similar reducing agent. The cytotoxic agent, with or without a linker moiety attached thereto, can then be added at a molar excess to the reduced antibody or antibody fragment in the presence of dimethyl sulfoxide (DMSO), or DMA. After conjugation, excess free cysteine may be added to quench unreacted agent. The cytotoxic agent, with or without a linker moiety having an amino-reactivable, or phenol- reactivable, or the others reactivable group (e.g. NHS, PFP) thereto, can be added directly at a molar excess to the antibody or antibody fragment in the presence of DMSO, or DMA to form a conjugate. The reaction mixture may then be purified through chromatography or buffer-exchanged into a suitable buffer, such as phosphate buffered saline (PBS), citrate buffer, or histine buffer.

The terms “cytotoxin” and “cytotoxic agent” refer to any molecule that inhibits or prevents the function of cells and/or causes destruction of cells (cell death), and/or exerts anti-proliferative effects. A cytotoxin or cytotoxic agent of an ADC also is referred to in the art as the “payload” of the ADC. A number of classes of cytotoxic agents are known in the art to have potential utility in ADC molecules and can be used in the ADC described herein. Such classes of cytotoxic agents include, for example, anti-microtubule agents (e.g., tubulysins, auristatins and maytansinoids), DNA minor groove binders (e.g. pyrrolobenzodiazepines (PBDs) or indolinobenzodiazepines (IGN) and their dimers), RNA polymerase II inhibitors (e.g., amatoxins), inhibitor of DNA topoisomerase I (e.g., camptothecins) and DNA alkylating agents (e.g., duocarmycin, CC-1065, pyrrolobenzodiazepine dimers or pseudodimers or indolinobenzodiazepine pseudodimers). Examples of specific cytotoxic agents that may be used in the ADC described herein include, but are not limited to, tubulysins, amanitins, auristatins, calicheamicin, camptothecins, daunomycins, doxorubicins, duocarmycins, dolastatins, enediynes, lexitropsins, taxanes, puromycins, maytansinoids, vinca alkaloids, and pyrrolobenzodiazepines (PBDs). More specifically, the cytotoxic agent may be, for example tubulysins, auristatins (AFP, MMAF, MMAE, AEB, AEVB, E), paclitaxels, docetaxels, CC-1065 (ducarmysin, DC1, DC4, CBI-dimers), camptothecins (SN-38, topotecans), morpholino-doxorubicin, rhizoxin, cyanomorpholino-doxorubicin, dolastatin-10, echinomycin, combretatstatin, chalicheamicin, maytansine (DM1, DM4, DM21), vinblastine, methotrexate, netropsin, or derivatives or analogs thereof. Cytotoxins suitable for use in ADCs are also described in, for example, International Patent Application Publication No. PCT/CN2021/128453.

In general, a chemotherapeutic agent or a functional compound can also be conjugated to the antibody of this invention. A chemotherapeutic agent or a functional compound is selected from the group consisting of:
a). an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). A plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; erbulins, a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid}; and Inhibitors of nicotinamide phosphoribosyltransferase (NAMPT);
e). A hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (comprising CB 1093, EB 1089 KH 1060, cholecalciferol, ergocalciferol); Photodynamic therapies: (comprising verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, neratinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group consisting of olaparib, niraparib, iniparib, talazoparib, veliparib, CEP 9722 (Cephalon's), E7016 (Eisai's), BGB-290 (BeiGene's), or 3-aminobcnzamide.
h). An antibiotic, selected from the group consisting of an enediync antibiotic (selected from the group consisting of calicheamicin, calicheamicin 71, 61, al or 01; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins andl-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((comprising Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; razoxane; rhizoxin; sizofiran; spirogermanium; tenuazonic acid; triaziquone; 2, 2′,2″-trichlorotriethylamine; trichothecenes (including the group consisting of T-2 toxin, verrucarin A, roridin A and anguidine); urethane, siRNA, antisense drugs;
(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.
(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem/cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefeapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). Glycopeptides: bleomycin, vancomycin (oritavancin, telavancin), teicoplanin (dalbavancin), ramoplanin;
g). Glycylcyclines: tigecycline;
h). p-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). Other antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides &_nucleotides: abacavir, aciclovir, adefovir, amdoxovir, apricitabine, brivudine, cidofovir, clevudine, dexelvucitabine, didanosine (ddl), elvucitabine, emtricitabine (FTC), entecavir, famciclovir, fluorouracil (5-FU), 3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.
(5). A radioisotope that can be selected from the group consisting of (radionuclides)³H, ¹¹C, ¹⁴C, ⁸F, ³²P, ³⁵S, ⁶⁴Cu, ⁶⁸Ga, ⁸⁶Y ⁹⁰Y⁹⁹Tc, In, ¹²³I, ¹²⁴I ¹²⁵I ¹³¹I, ¹³³Xe, ¹⁷⁷L, ²⁰³Pb, ²¹²Pb, ²¹At, ²¹³Bi, ²²⁴Ra and ²²⁵Ac. The metal radionuclides, ⁶⁴Cu, ⁶⁸Ga, ⁸⁶Y ⁹⁰Y⁹⁹Tc, ¹¹¹In, ¹³³Xe, ¹⁷⁷L, ²⁰³Pb, ²¹²Pb, ²¹¹At, ²¹³Bi, ²²⁴Ra and ²²⁵Ac linked to an antibody through a chelator and a linker Li of this patent application. The chelator is selected from DOTA, TETA, NOTA, NETA, DTPA, HBED, SHBED, and their structures are illustrated below:

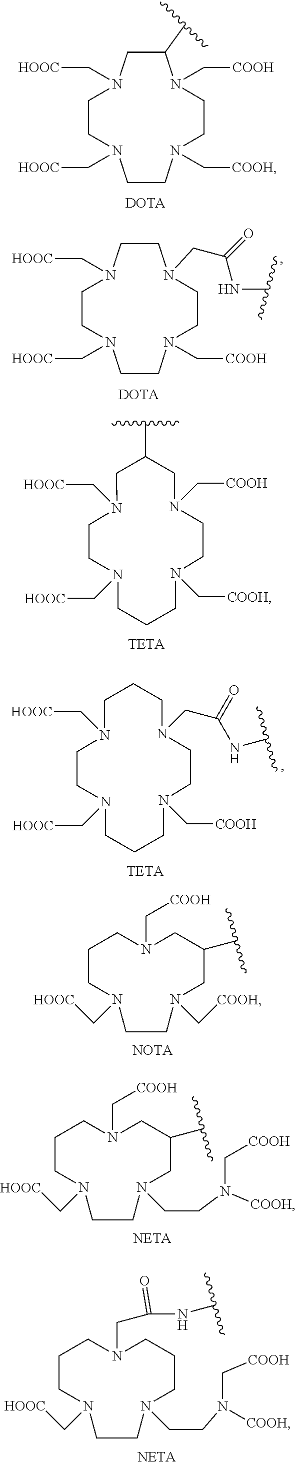

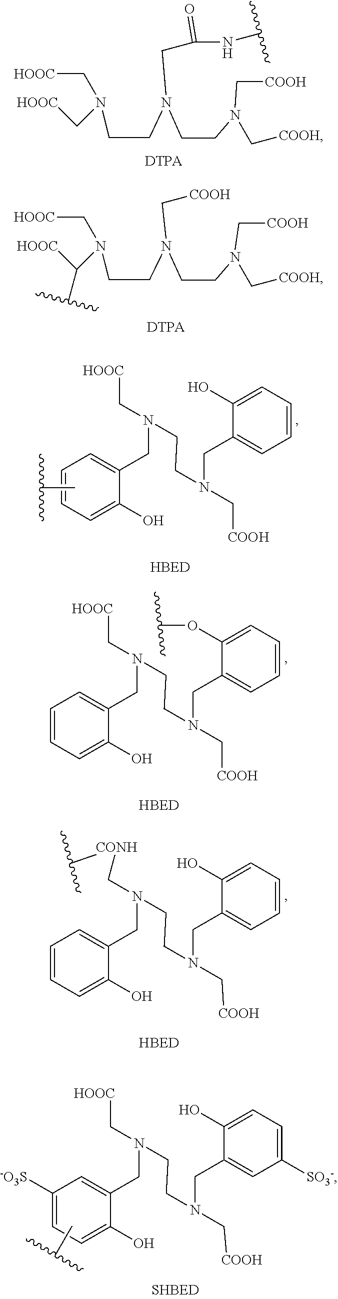

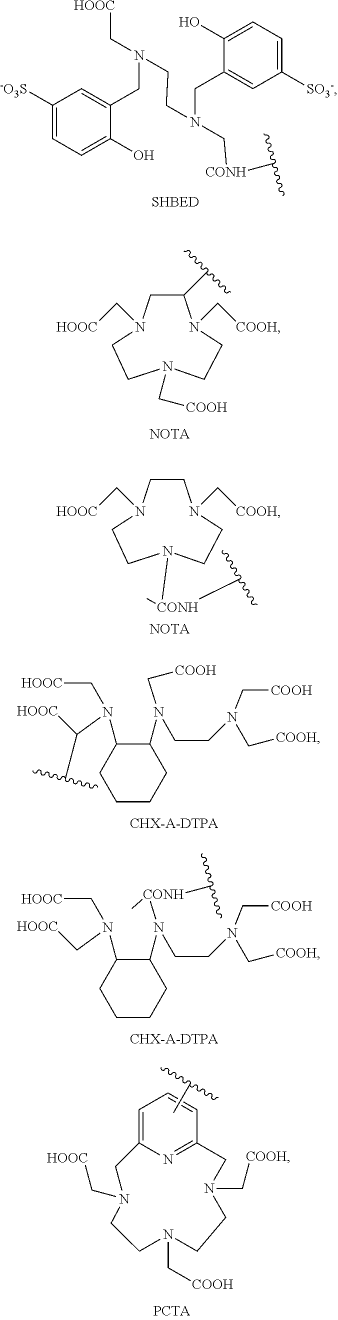

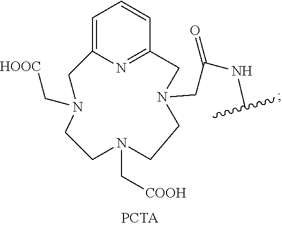 [SMILES: *NC(=O)CN1CCN(CC(=O)O)CCN(CC(=O)O)Cc2cccc(n2)C1]

(6). A chromophore molecule, which is capable of absorbing UV light, florescent light, IR light, near IR light, visual light; A class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; Non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170). Acridine derivatives (comprising proflavin, acridine orange, acridine yellow). Arylmethine derivatives (comprising auramine, crystal violet, malachite green). Tetrapyrrole derivatives (comprising porphin, phthalocyanine, bilirubin); Any analogs and derivatives of the following fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin(PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, Propidiumlodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; A fluorophore compound: comprising DCFH (2′7′Dichorodihydro-fluorescein, oxidized form), DHR (Dihydrorhodamine 123, oxidized form, light catalyzes oxidation), Fluo-3 (AM ester. pH >6), Fluo-4 (AM ester. pH 7.2), Indo-1 (AM ester, low/high calcium (Ca2+)), SNARF(pH 6/9), Allophycocyanin(APC), AmCyan1 (tetramer, Clontech), AsRed2 (tetramer, Clontech), Azami Green (monomer), Azurite, B-phycoerythrin (BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRedl, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreenl (tetramer), ZsYellowl (tetramer) and their derivatives.
(7). The cell-binding ligands or receptor agonists, which can be selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting ofPyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N1O-diethyl)-homoArg-Pro-D-Ala-NH₂); Pattern Recognition Receptor (PRR₈), selected from the group consisting of Toll-like receptors' (TLR₈) ligands, C-type lectins and Nodlike Receptors' (NLR₈) ligands; Calcitonin receptor agonists; integrin receptors' and their receptor subtypes' (selected from the group consisting of αᵥβ₁, αᵥβ₃, αᵥβ₅, αᵥβ₆, α₆β₄, α7β₁, α_{L}β₁, α_{llb}β₃) agonists (selected from the group consisting of GRGDSPK, cyclo(RGDfV) (LI) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists; an immunotherapeutical short antibody-like protein, siRNA or DNA molecule.
(8). The pharmaceutically acceptable salts, acids, derivatives, hydrate or hydrated salt; or a crystalline structure; or an optical isomer, racemate, diastereomer or enantiomer of any of the above drugs.

In another embodiment, the drug D can be polyalkylene glycols that are used for extending the half-life of the cell-binding antibody, or antibody molecule when administered to a mammal. Polyalkylene glycols include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodoacetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones).

In certain such embodiments, the polyalkylene glycol has a molecular weight of from about 10 Daltons to about 200 kDa, preferably about 88 Da to about 40 kDa; two branches each with a molecular weight of about 88 Da to about 40 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428, 128; 5,621, 039; 5,622,986; 5,643,575; 5,728,560; 5,730,990; 5,738,846; 5,811,076; 5, 824, 701; 5,840,900; 5,880, 131; 5,900,402; 5,902,588; 5,919,455; 5,951,974; 5,965, 119; 5, 965, 566; 5,969, 040; 5,981,709; 6,011,042; 6,042,822; 6, 113, 906; 6, 127, 355; 6, 132, 713; 6, 177, 087, and 6, 180, 095.

In yet another embodiment, D is more preferably a potent cytotoxic agent, selected from a tubulysin and its analogs, a maytansinoid and its analogs, a taxanoid (taxane) and its analogs, a CC-1065 and its analogs, a daunorubicin or doxorubicin and its analogs, an amatoxin and its analogs, a benzodiazepine dimer (e.g., dimers of pyrrolobenzodiazepine (PBD), tomaymycin, anthramycin, indolinobenzodiazepines, imidazobenzothiadiazepines, or oxazolidinobenzodiazepines) and their analogs, a calicheamicin and the enediyne antibiotic and their analogs, an actinomycin and its analogs, an azaserine and its analogs, a bleomycin and its analogs, an epirubicin and its analogs, a tamoxifen and its analogs, an idarubicin and its analogs, a dolastatin and its analogs, an auristatin (including monomethyl auristatin E (MMAE), MMAF, auristatin PYE, auristatin TP, Auristatins 2-AQ, 6-AQ, EB (AEB), and EFP (AEFP)) and its analogs, a combretastatin, a duocarmycin and its analogs, a camptothecin, a geldanamycin and its analogs, a methotrexate and its analogs, a thiotepa and its analogs, a vindesine and its analogs, a vincristine and its analogs, a hemiasterlin and its analogs, a nazumamide and its analogs, a spliceostatin, a pladienolide, a microginin and its analogs, a radiosumin and its analogs, an alterobactin and its analogs, a microsclerodermin and its analogs, a theonellamide and its analogs, an esperamicin and its analogs, PNU-159682 and its analogs, a protein kinase inhibitor, a MEK inhibitor, a KSP inhibitor, a nicotinamide phosphoribosyltransferase (NAMPT) inhibitor, an immunotoxin, a cell receptor agonist, a cell stimulating molecule or intracellular signaling molecule, one, two or more DNA, RNA, mRNA, small interfering RNA (siRNA), microRNA (miRNA), and PIWI interacting RNAs (piRNA), and stereoisomers, isosteres, analogs, or derivatives thereof;.

In certain such embodiments, tubulysin and its analogs are well known in the art and can be isolated from natural sources according to known methods or prepared synthetically according to known methods (e. g. Balasubramanian, R., et al. J. Med. Chem., 2009, 52, 238-40; Wipf, P., et al. Org. Lett., 2004, 6, 4057-60; Pando, 0., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, 0., et al. Org. Lett., 2009, 11 (24), 5567-9; Wipf, P., et al. Org. Lett., 2007, 9 (8), 1605-7; Friestad, G. K., Org. Lett., 2004, 6, 3249-52; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49: 4809-12; Chai, Y., et al., Chem Biol, 2010, 17: 296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; Patent applications: Zanda, M., et al, Can. Pat. Appl. CA 2710693 (2011); Chai, Y., et al. Eur. Pat. Appl. 2174947 (2010), WO 2010034724; Leamon, C. et al, WO2010033733, WO 2009002993; Ellman, J., et al, PCT WO2009134279; WO 2009012958, US appl. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. Pat.Appl. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., US appl. 20110027274. The preferred structures of tubulysins for conjugation of cell binding molecules through process of the present patent application are described in the patent application of PCT/IB2012/053554.

Tubulysin analog having the following formula (IV):

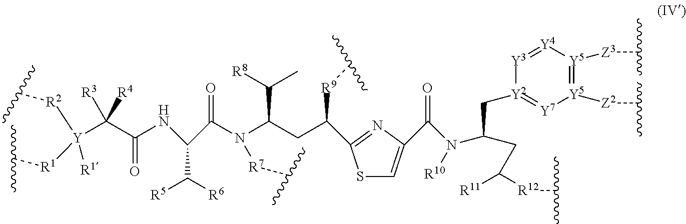

or a pharmaceutically acceptable salt, hydrates, or hydrated salt; or a polymorphic crystalline structure; or an optical isomer, racemate, diastereomer or enantiomer thereof,
wherein [symbol] is a linkage site that either one or two of them can link to L₁ and/or L₂ independently; when two of “ [symbol] ” link to both L₁ and L₂, R¹ and R², or Z² and Z³ are preferably the dual linkage sites;
wherein R¹, R^{1′}, R², R³, and R⁴ are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl; or R¹R², R¹R₃, R²R₃, R³R⁴, or RSR⁶ form a 3~7 membered carbocyclic, cycloalkyl, heterocyclic, heterocycloalkyl, aromatic or heteroaromatic ring system; R¹ and R² can be independently absent when they link to L₁ or L₂ independently or simultaneously, Y¹ is N or CH;
wherein R⁵, R⁶, R⁸, R¹⁰ and R¹¹ are independently H, or C₁-C₄ alkyl or heteroalkyl; wherein R⁷ is independently H, R¹⁴, —R¹⁴C(═O)X¹R¹⁵; or —R¹⁴X¹R⁵; X¹ is O, S, S—S, NH, CH₂ or NR¹⁴—
wherein R⁹ is selected from H, OH, ═O, —OR¹⁴, —OC(═O)R¹⁴, —OC(═O)NHR¹⁴, —OC(═O) NR¹⁴R¹⁵, OP(═O)(OR¹⁴)₂, —OC(═O)NR¹⁴R¹⁵, or OR¹⁴OP(═O)(OR¹⁵)₂; when R⁹ links L₁ or L₂, R⁹ is —O—, —OC(═O)NH— or —OC(═O)N(R¹⁴)—;
wherein R¹¹ is independently H, R¹⁴, —R¹⁴C(═O)R¹⁵, —R¹⁴C(═O)X²R¹⁵, wherein X² is —O—, —S—, —NH—, or —N(R¹⁴)—;
wherein R¹² is —COOH, —COSH, —CONH₂, CONHNH₂, CONHNHR¹⁵, —CONH(R¹⁵), —COOR¹⁵, —R¹⁵COR¹⁶, —R¹⁵COOR¹⁶, —R¹⁵C(O)NH₂, —R¹⁵C(O)NHR¹⁶, —COSR¹⁵, R¹⁵S(═O)₂R¹⁶, —R¹⁵P(═O)(OR¹⁷)₂, —R¹⁵OP(═O)(OR¹⁷)₂, —COOCH₂OP(═O)(OR¹⁷)₂, —COX²SO₂R¹⁷, —COOR¹⁵X²R¹⁶, tetrazole, imidazole, or triazole, where X² is —O—, —S—, —NH—, —N(R¹⁵)—, —O—R¹⁵—, —S—R¹⁵—, CH₂ or —NHR¹⁵—; when R¹² links L₁ or L₂, R¹² is —C(O)O—, —C(O)NH—, —C(═O)NHS(O)₂R¹⁵— or —C(═O)N(R¹⁵)—
R¹³ and R¹⁴ are independently C₁-C₈alkyl, heteroalkyl; C₂-C₈ of alkenyl, alkynyl, heteroalkyl, heterocycloalkyl; C₃-C₈ of aryl, Ar-alkyl;
Z² and Z³ are independently H, O, S, NH, N(R¹⁵), NHNH, —OH, —SH, —NH₂, NH, NHNH₂, —NH(R¹⁵), —OR¹⁵, CO, —COX², —COX²R¹⁶, R¹⁷, F, Cl, Br, I, SR¹⁶, NR¹⁶R¹⁷, N═NR¹⁶, N═R¹⁶, NO₂, SOR¹⁶R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷;—O—(C₄-C₁₂ glycoside), —N—(C₄-C₁₂ glycoside); C₁-C₈alkyl, heteroalkyl; C₂-C₅ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)₁₋₈, or CO(Aa)₁₋₈ (which are respectively N-terminal or C-terminal 1-8 the same or different amino acids)), or polyethyleneoxy unit of formula (OCH₂CH₂)ₚ or (OCH₂CH(CH₃))ₚ, wherein p is an integer from 0 to about 1000, or combination of above groups thereof; X² is O, S, S—S, NH, CH₂, OH, SH, NH₂, CHR¹⁵ or NR″;
R¹⁵, R¹⁶ and R¹⁷ are independently H, C₁-C₈alkyl, heteroalkyl; C₂-C₅ of alkenyl, alkynyl, heteroalkyl, heterocycloalkyl; C₃-C₈ of aryl, Ar-alkyl, cycloalkyl, heteroalkylcycloalkyl, alkylcarbonyl, heteroaryl, alkylcarbonyl, or Na⁺, K⁺, Cs⁺, L-i⁺, Ca²⁺ , Mg⁺, Zn²⁺, N⁺(R¹)(R²)(R³) (R⁴), HN⁺(C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N (R1), thus Y², Y⁴, Y⁵, Y⁶ and Y⁷form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y⁴, Y⁵, Y⁶ and Y⁷ form aromatic ring of benzene, pyridine, pyridazine, pyrimidine, pyrazine, triazine, tetrazine, pentazine;

Examples of the structures of the tubulysin analogs are shown below:

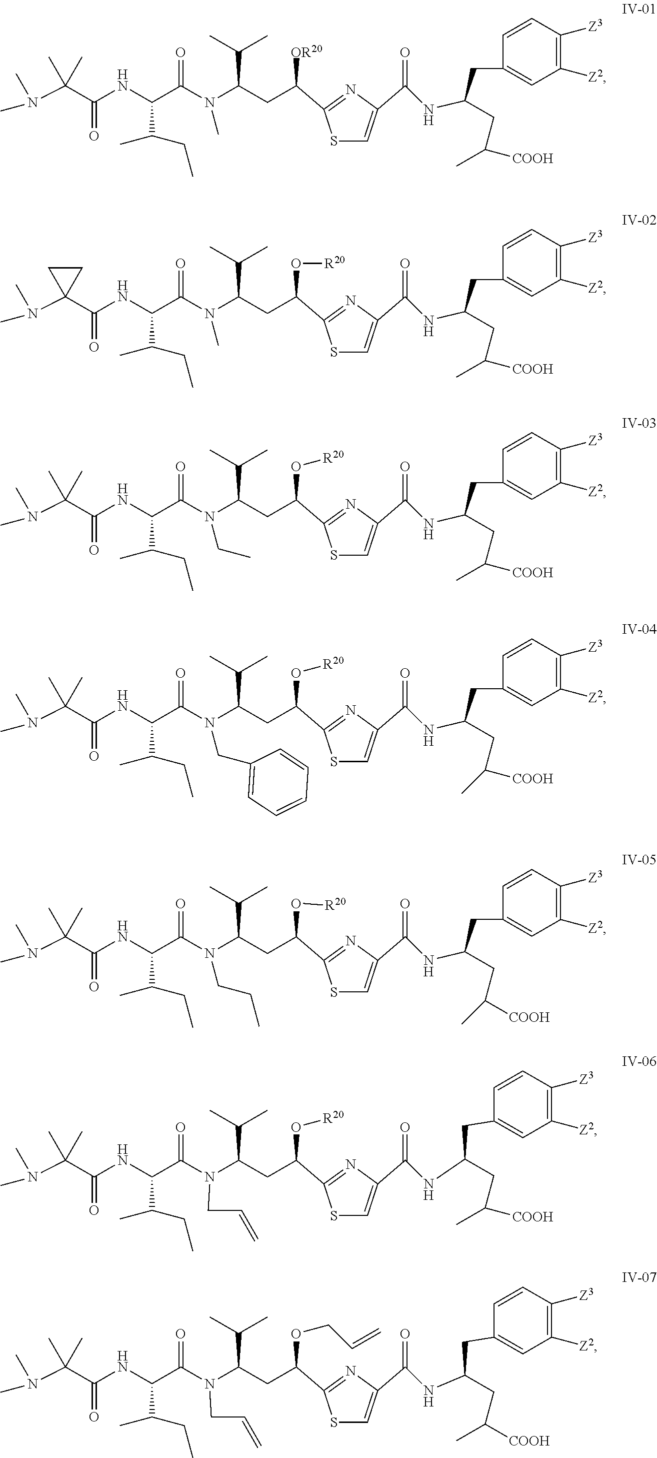

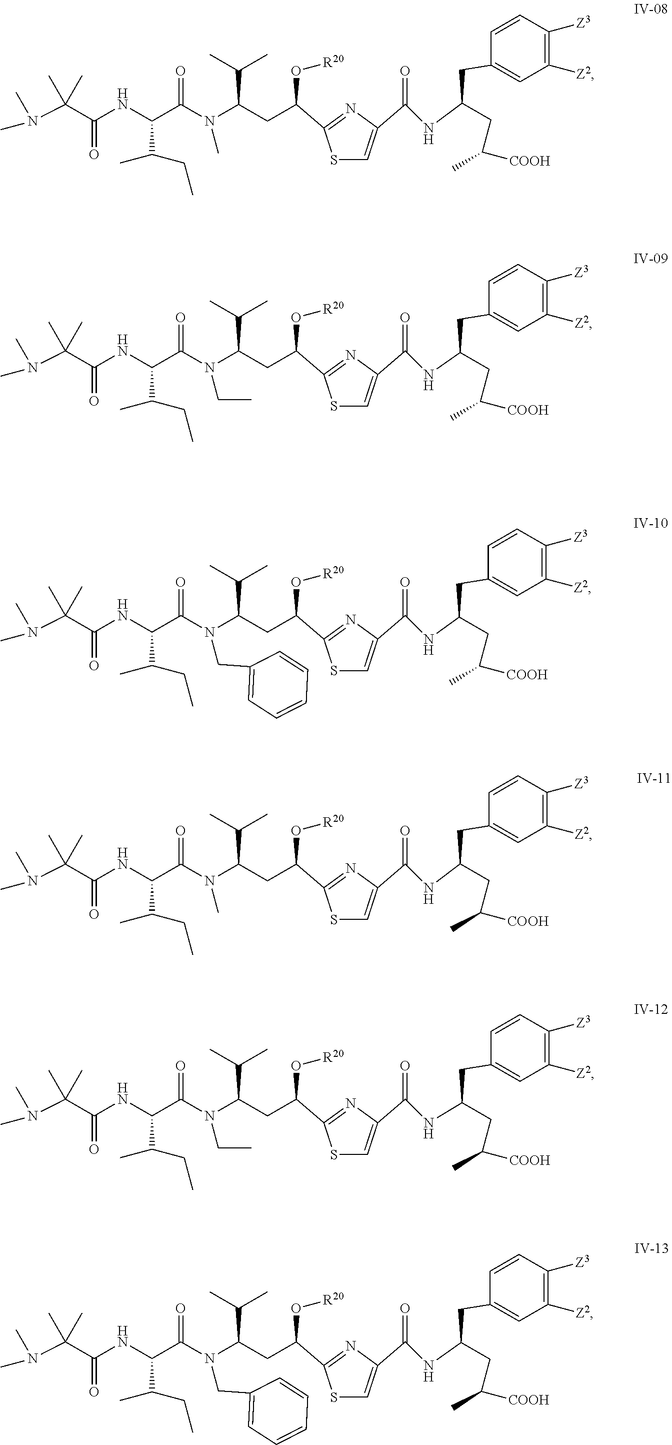

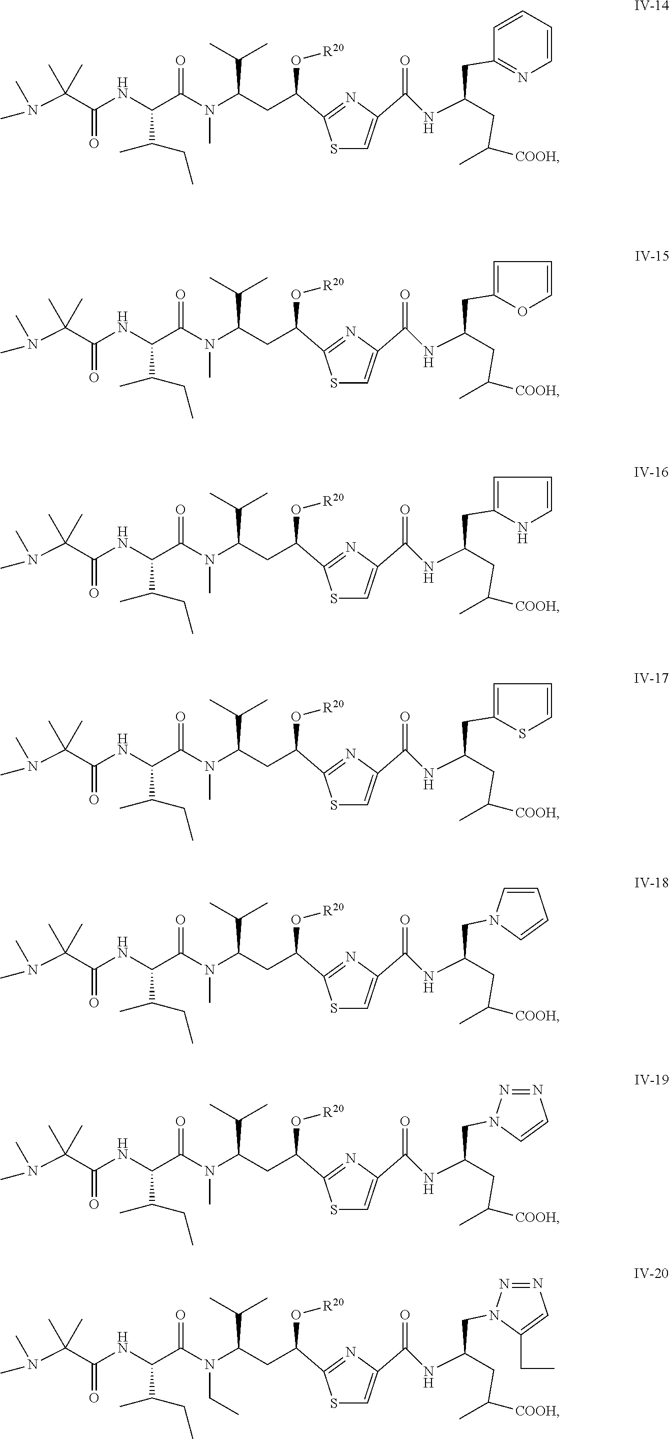

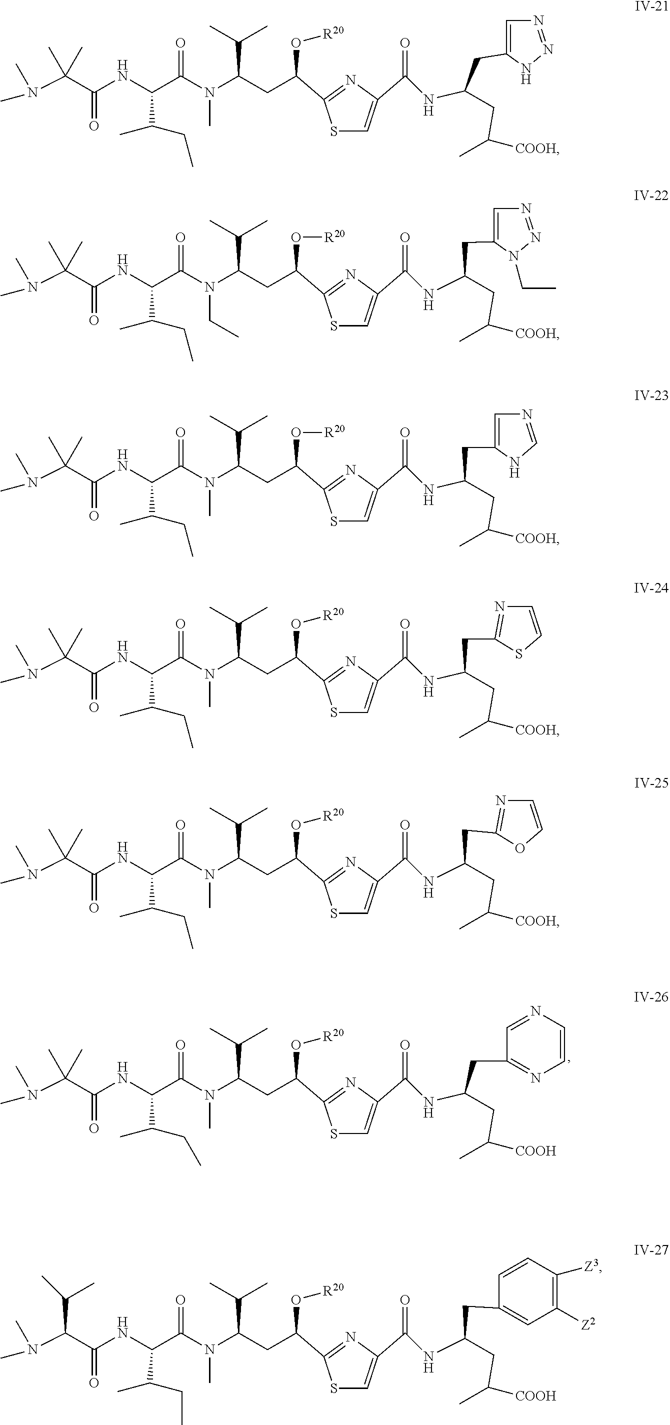

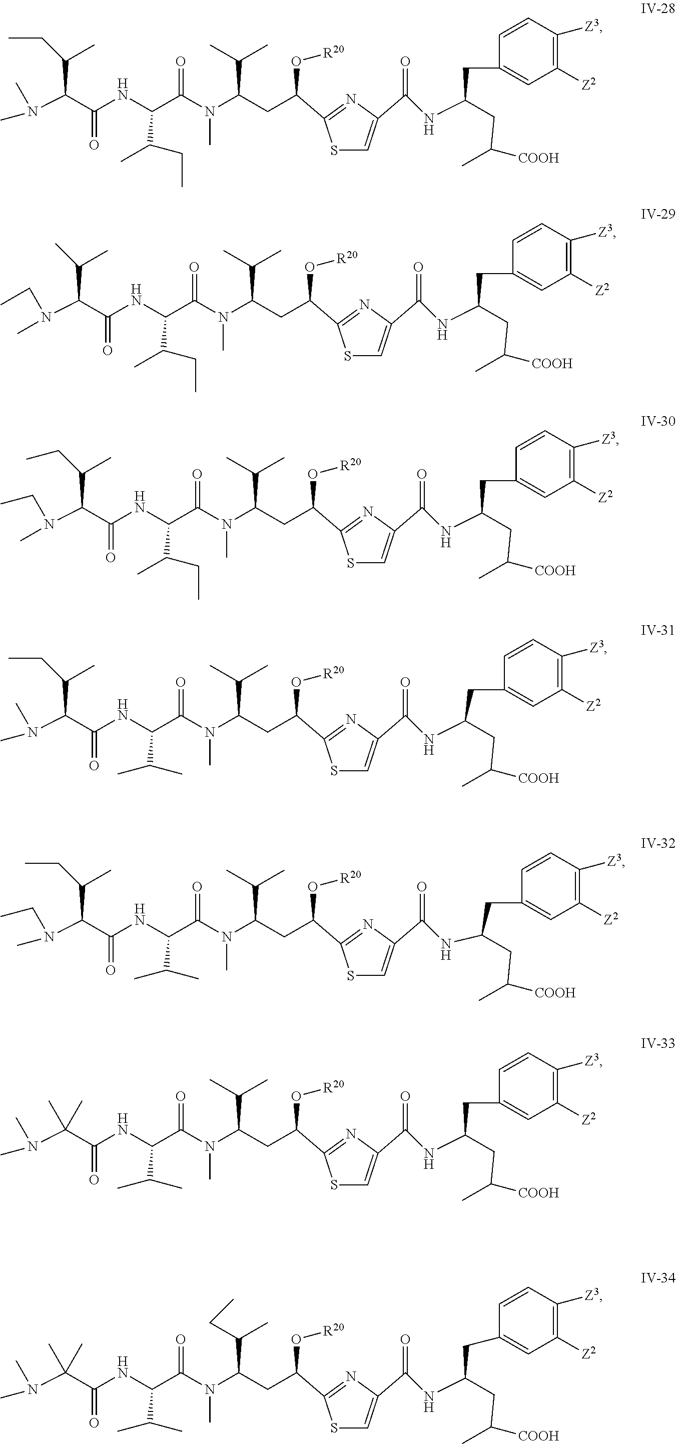

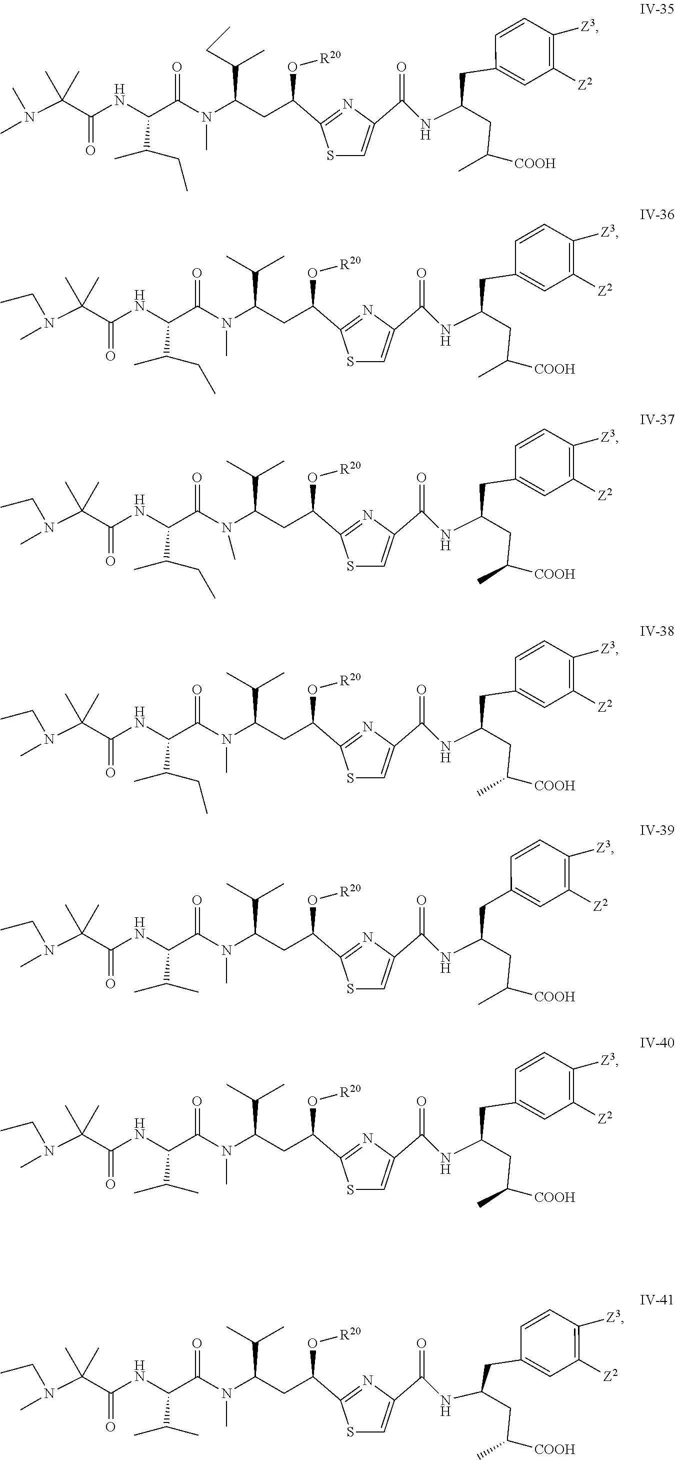

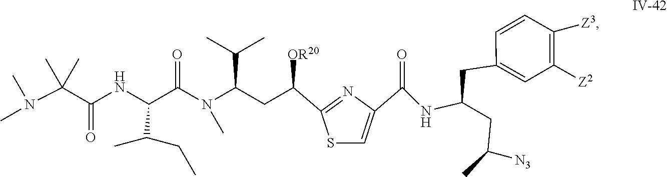

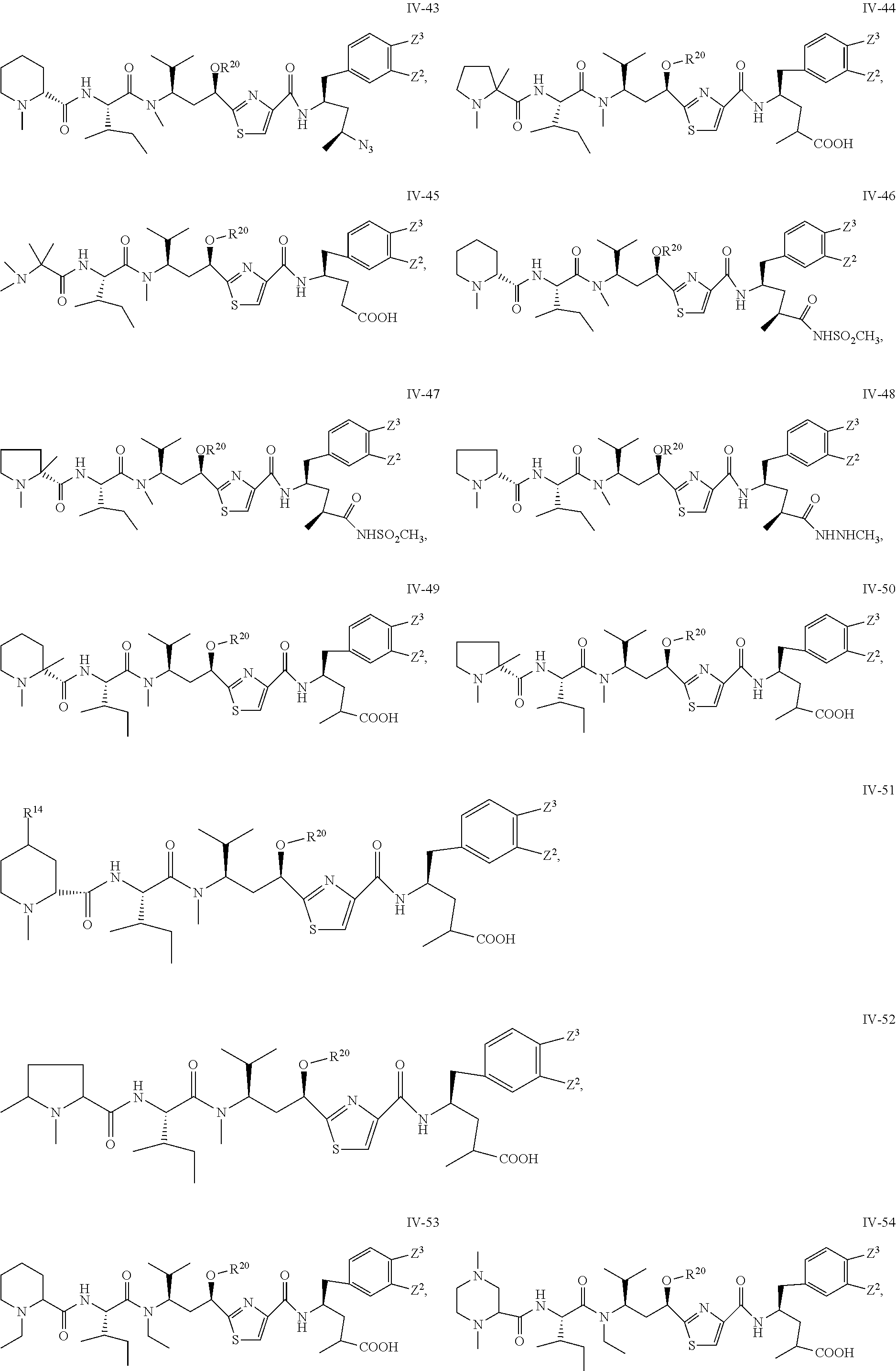

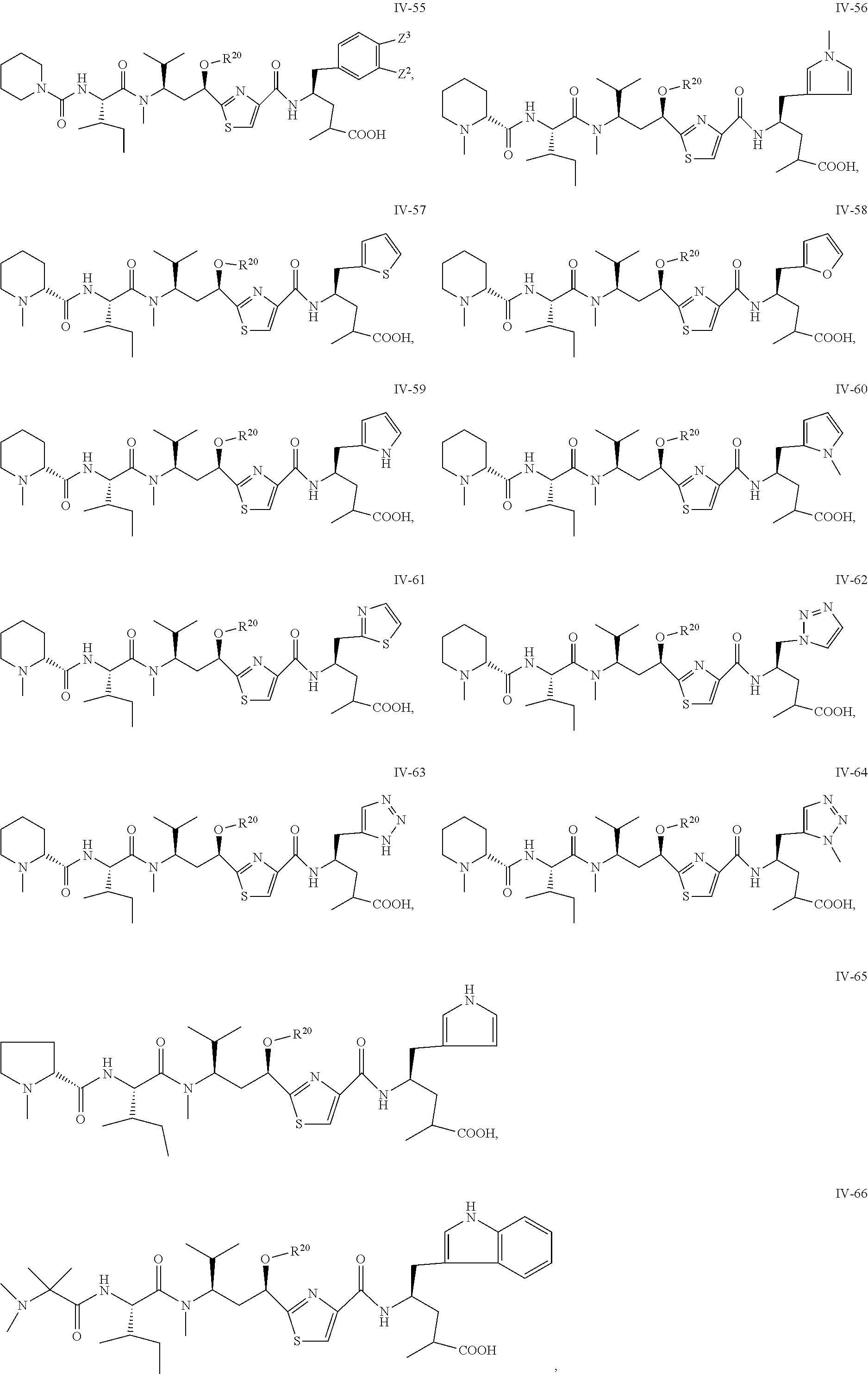

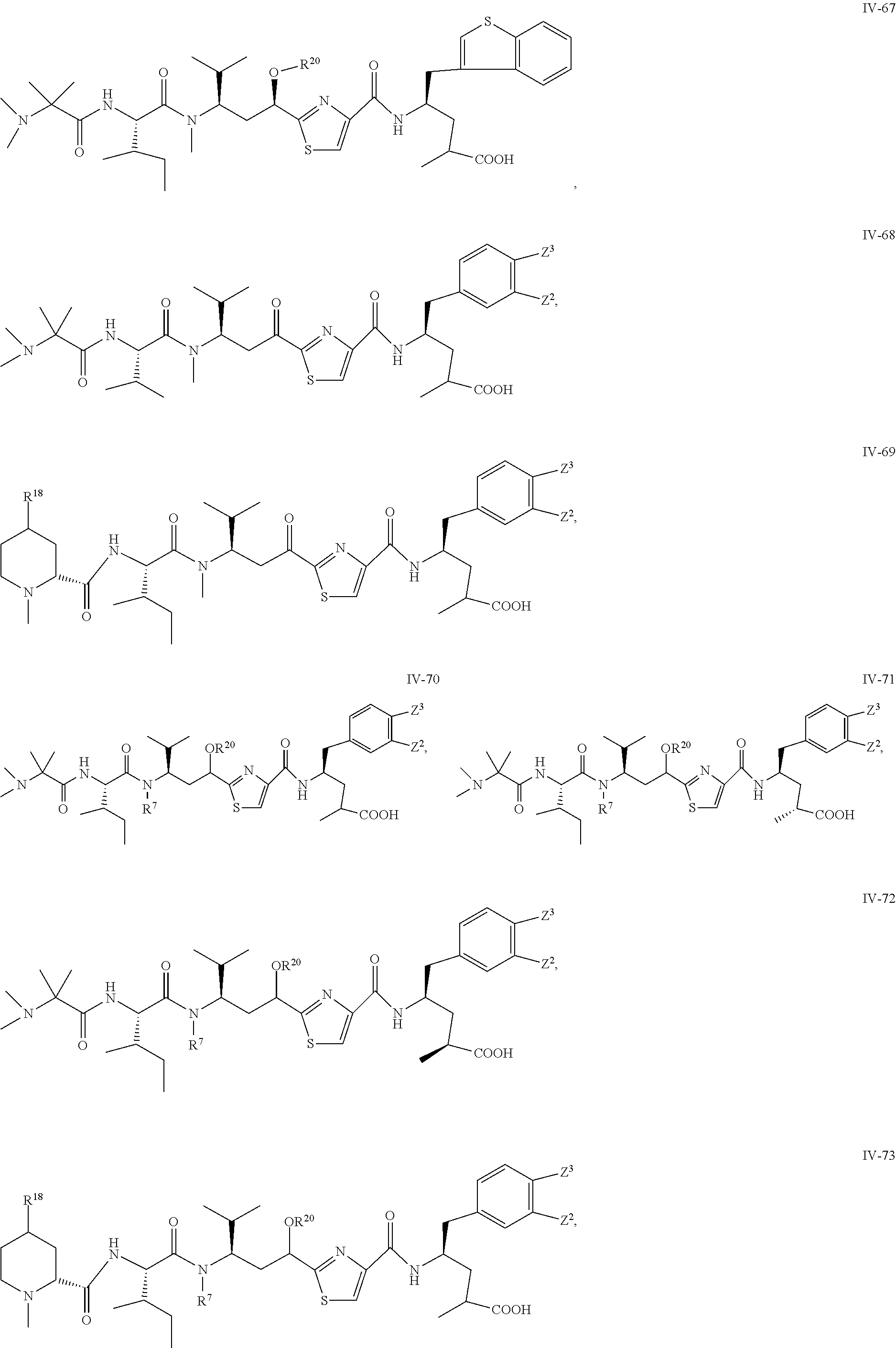

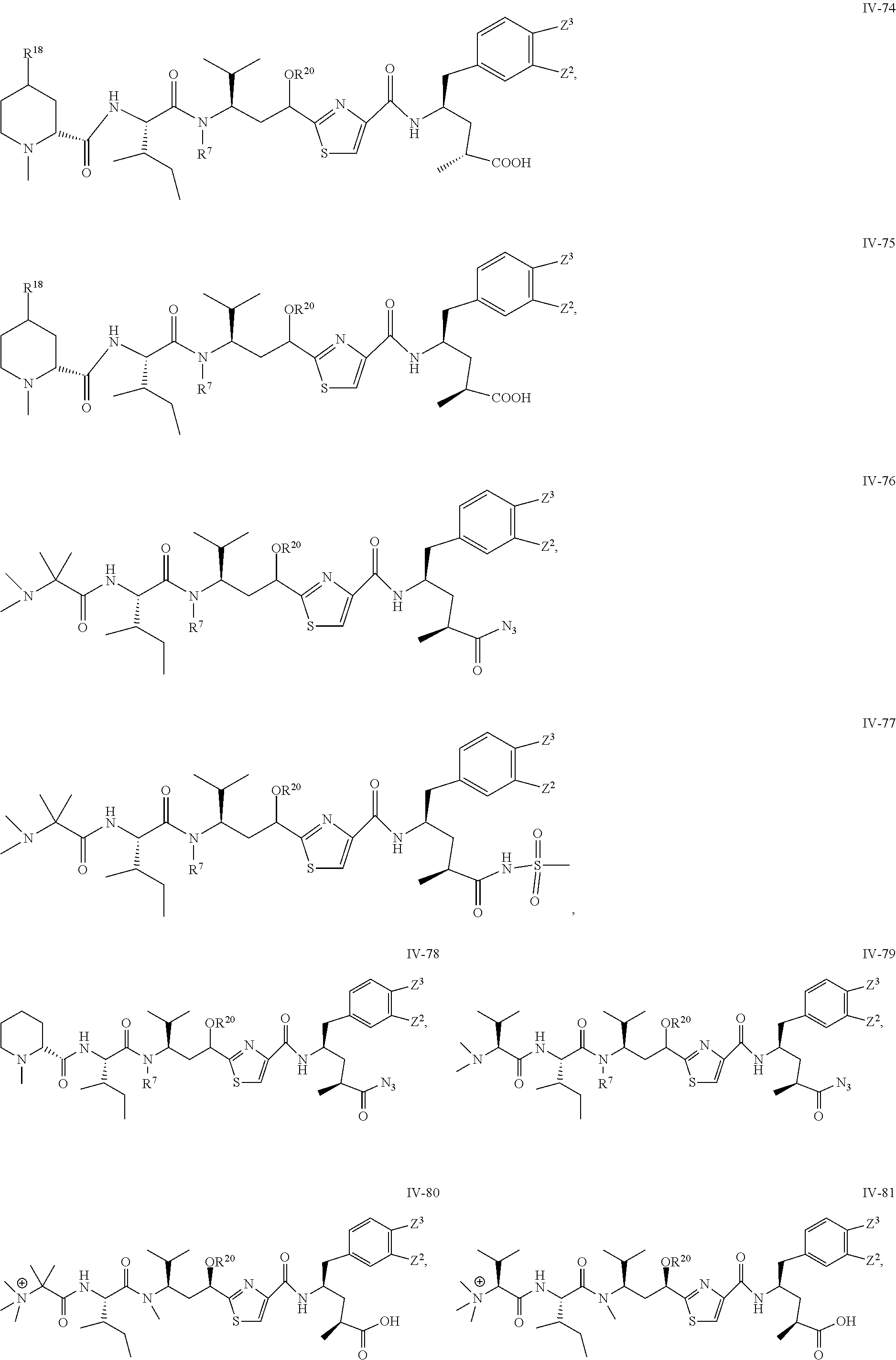

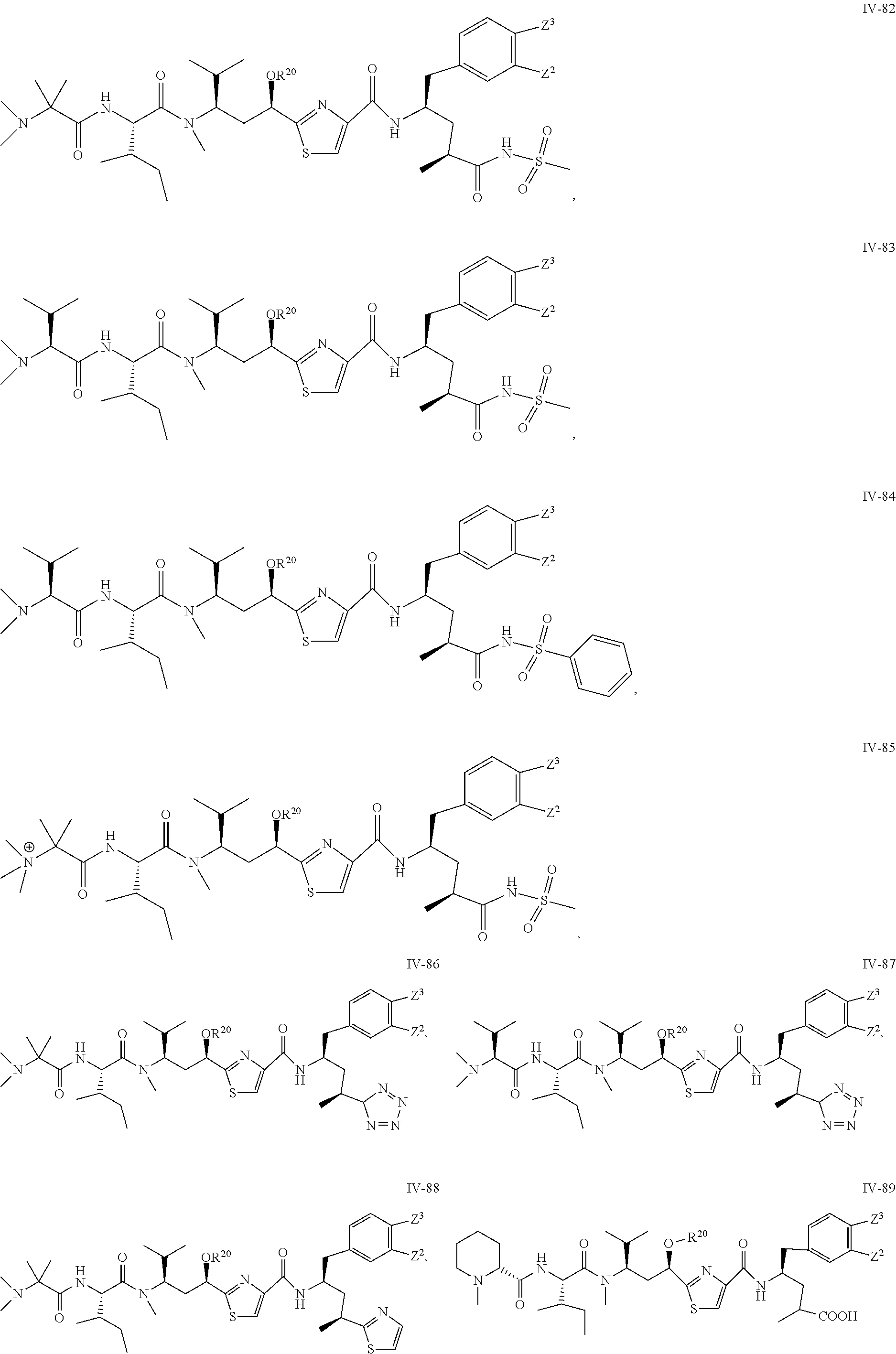

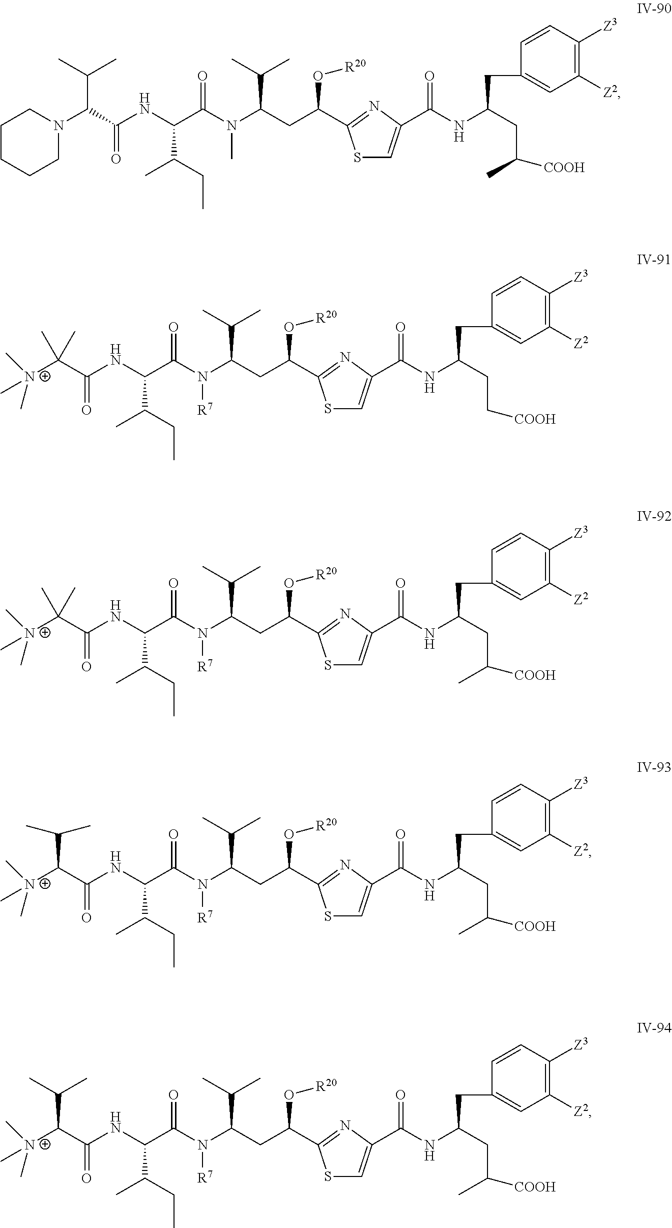

wherein R²⁰ is H; C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); or 1-8 carbon atoms of carboxylate, esters, ether, or amide; or 1-8 amino acids; or polyethyleneoxy unit of formula (OCH₂CH₂)ₚ or (OCH₂CH(CH₃))ₚ, wherein p is an integer from 0 to about 1000; or R²⁰ is absent and the oxygen forms a ketone, or combination above groups thereof;
Z³ and Z³ are independently H, OH, NH₂, O, NH, COOH, COO, C(O), C(O), C(O)N H, C(O)N H₂, R¹⁸, O CH₂OP(O)(OR¹⁸)₂, O C(O)OP(O)(OR¹⁸)₂, O PO(OR¹⁸)₂, NHPO(OR¹⁸)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R″);
R¹⁹ is H, OH, NH₂, OSO₂(OR¹⁸), XCH₂OP(O)(OR¹⁸)₂, XPO(OR¹⁸)₂, XC(O)OP(O)(OR¹⁸)₂, XC(O)R¹⁸, XC(O)NHR¹⁸, C₁-C₈ alkyl or carboxylate; C₂-C₅ alkenyl, alkynyl, alkylcycloalkyl, heterocycloalkyl; C₃-C₈ aryl or alkylcarbonyl; or pharmaceutical salts;
X is O, S, NH, NHNH, or CH₂;
R⁷ is defined the same above; wherein the linkage sites, “ [symbol] ” in formula IV-01- IV-79 are the same indication according to formula (IV).

In certain such embodiments, calicheamicins and their related enediyne antibiotics that are described in: Nicolaou, K. C. et al, Science 1992, 256, 1172-1178; Proc. Natl. Acad. Sci USA. 1993, 90, 5881-8), U.S. Pat. Nos. 4,970,198; 5, 053, 394; 5, 108, 912; 5, 264, 586; 5, 384, 412; 5, 606, 040; 5,712,374; 5,714,586; 5,739, 116; 5,770,701; 5,770,710; 5,773,001; 5,877,296; 6,015, 562; 6, 124, 310; 8, 153, 768. Exemplary enediynes include, but are not limited to, calicheamicin, esperamicin, uncialamicin, dynemicin, and their derivatives. The structure of calicheamicins is preferred the following formula:

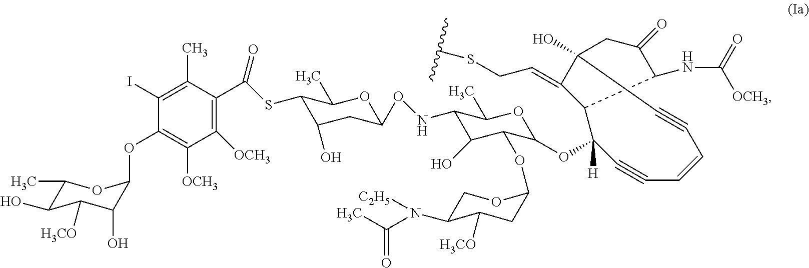 [SMILES: *SC/C=C1\[C@@H]2C(NC(=O)OC)C(=O)C[C@@]1(O)C#C/C=C\C#C[C@@H]2OC1OC(C)C(NOC2CC(O)C(SC(=O)c3c(C)c(I)c(OC4OC(C)C(O)C(OC)C4O)c(OC)c3OC)C(C)O2)C(O)C1OC1CC(OC)C(N(CC)C(C)=O)CO1]

or an isotope of a chemical element, or a pharmaceutically acceptable salt, hydrates, or hydrated salt; or a polymorphic crystalline structure; or an optical isomer, racemate, diastereomer or enantiomer thereof,
wherein [symbol] is the site linked to L₁ or L₂;

In certain such embodiments, Geldanamycins are benzoquinone ansamycin antibiotic that bind to Hsp90 (Heat Shock Protein 90) and have been used antitumor drugs. Exemplary geldanamycins include, but are not limited to, 17-AAG (17-N-Allylamino-17-Demethoxygeldanamycin) and 17-DMAG (17-Dimethylamino-ethylamino-17-demethoxygeldanamycin), having the following formula:

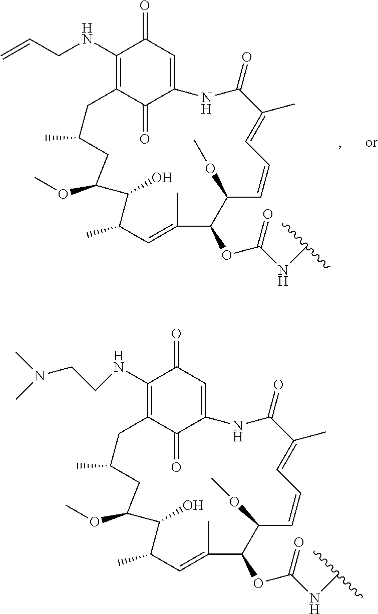

wherein [symbol] is the site linked to L₁ or L₂;

In certain such embodiments, Maytansines or their derivatives maytansinoids inhibit cell proliferation by inhibiting the mcirotubules formation during mitosis through inhibition of polymerization of tubulin. See Remillard et al., Science 189:1002-1005 (1975). Exemplary maytansines and maytansinoids include, but are not limited to, mertansines (DM1, DM4), maytansinol and its derivatives as well as ansamitocin. Maytansinoids are described in U.S. Pat. Nos. 4,256,746, 4, 361, 650, 4, 307, 016, 4, 294, 757, 4, 294, 757, 4, 371, 533, 4, 424, 219, 4, 331, 598, 4, 450, 254, 4, 364, 866, 4, 313, 946, 4, 315, 929 4, 362, 663, 4, 322, 348, 4, 371, 533, 4, 424, 219, 5, 208, 020, 5, 416, 064, 5, 208, 020; 5, 416, 064; 6, 333.410; 6, 441, 163; 6, 716, 821, 7, 276, 497, 7, 301, 019, 7, 303, 749, 7, 368, 565, 7, 411, 063, 7, 851, 432, and 8, 163, 888. The structure of maytansinoids is preferred the following formula:

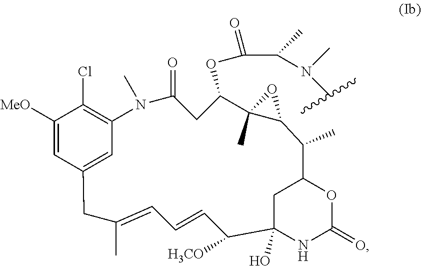

wherein [symbol] is the site linked to L₁ or L₂.

In certain such embodiments, camptothecins (CPTs) and its derivatives, which are topoisomerase I inhibitors to prevent DNA re-ligation and therefore to causes DNA damage resulting in apoptosis, are described in: Shang, X. F. et al, Med Res Rev. 2018, 38(3):775-828; Botella, P. and Rivero-Buceta, E. J Control Release. 2017, 247:28-54; Martino, E. et al, Bioorg Med Chem Lett. 2017, 27(4):701-707; Lu, A., et al, Acta Pharmacol Sin 2007, 28(2): 307-314. It includes SN-38, Topotecan, Irinotecan (CPT-1 1), Silatecan (DB-67, AR-67), Cositecan (BNP-1350), Etirinotecan, Exatecan, Lurtotecan, Gimatecan (ST1481), Belotecan (CKD-602), Rubitecan and several others (Shang, X. F. et al, Med Res Rev. 2018, 38(3):775-828). So far three CPT analogues, topotecan, irinotecan, and belotecan have been approved and are used in cancer chemotherapy (Palakurthi, S., Expert Opin Drug Deliv. 2015; 12(12):1911-21; Shang, X. F. et al, Med Res Rev. 2018, 38(3):775-828) and both SN-38 and Exatecan have been successfully used as payloads for ADC conjugates in the clinical trials (Ocean, A. J. et al, Cancer. 2017, 123(19): 3843-3854; Starodub, A. N., et al, Clin Cancer Res. 2015, 21(17): 3870-8; Cardillo, T. M., et al, Bioconjug Chem. 2015, 26(5): 919-31; Ogitani, Y. et al, Bioorg Med Chem Lett. 2016, 26(20): 5069-5072; Takegawa, N. et al, Int J Cancer. 2017 Oct. 15; 141(8):1682-1689. US patents 7, 591, 994; 7, 999, 083, 8, 080, 250, 8, 268, 317; US patent applications 20130090458, 20140099258, 20150297748, 20160279259).

The structure of Camptothecin (CPT) used for the patent is illustrated below formula:

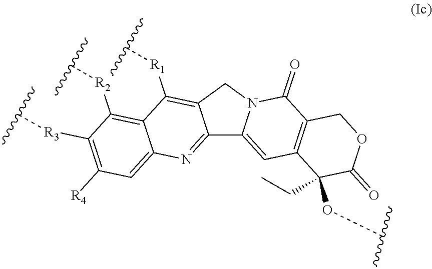

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R₁, R₂ and R₄ are independently selected from H, F, Cl, Br, CN, NO₂, C₁-C₈ alkyl; O—C₁-C₈ alkyl; NH—C₁~C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, carbonate, urea, or carbamate; R₃ is H, OH, NH₂, C₁-C₈alkyl; O—C₁~C₈ alkyl; NH—C₁~C₈ alkyl; C₂-C₈ of heteroalkyl, alkylcycloalkyl, heterocycloalkyl; or 2-8 carbon atoms of esters, ether, amide, carbonate, urea, or carbamate; or R₁R2, R₂R₃ and R₃R₄ independently form a 5~7 membered carbocyclic, heterocyclic, heterocycloalkyl, aromatic or heteroaromatic ring system; [symbol] is the site in the molecule that can be linked to L₁ or L₂.

The structures of camptothecins are preferred the following formula:

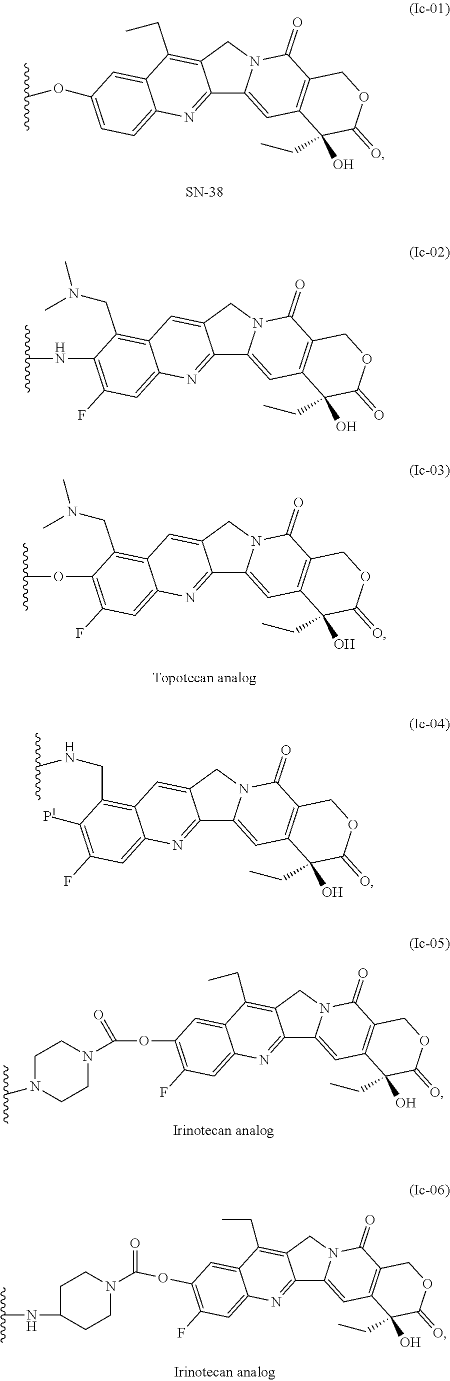

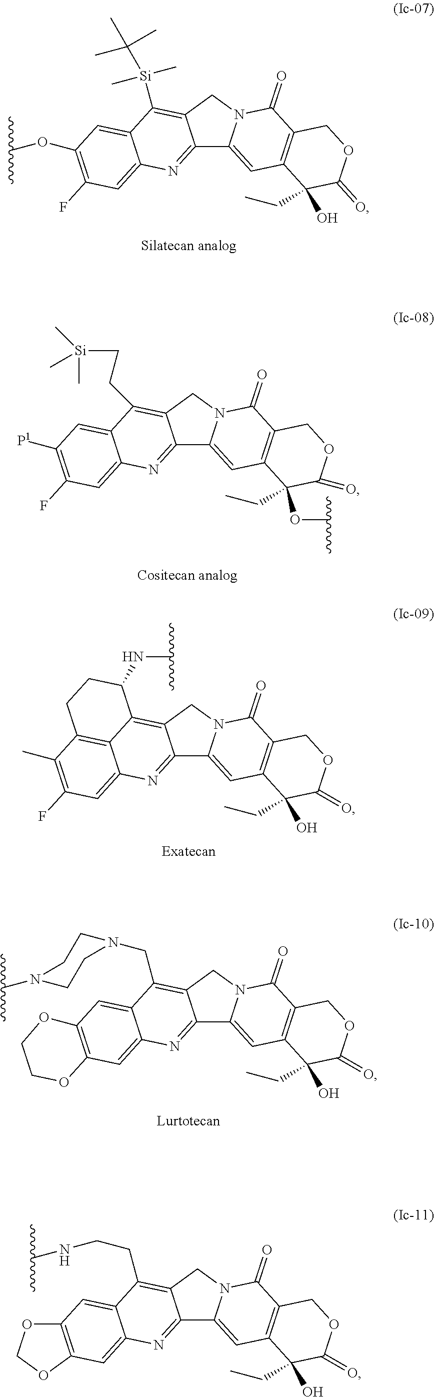

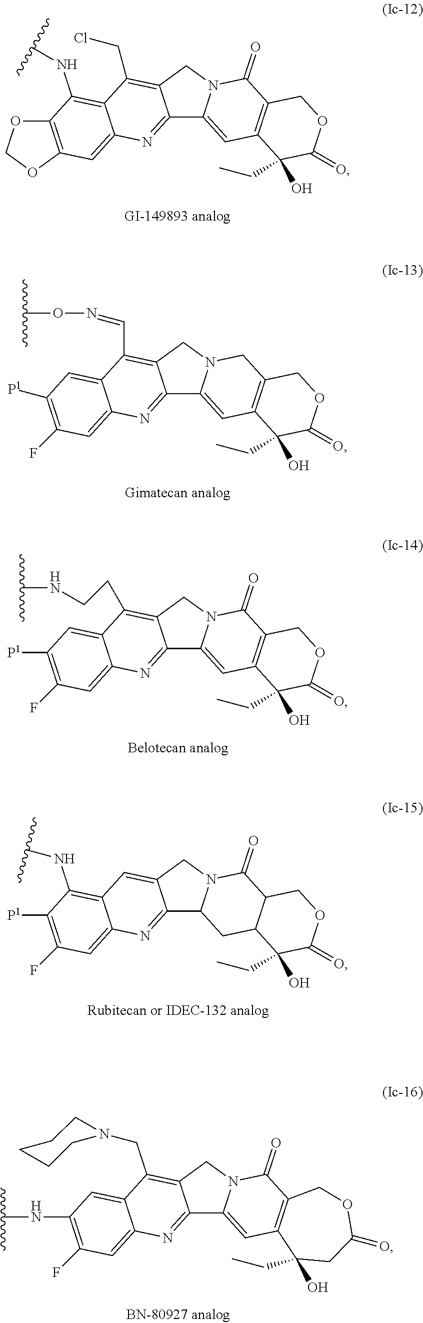

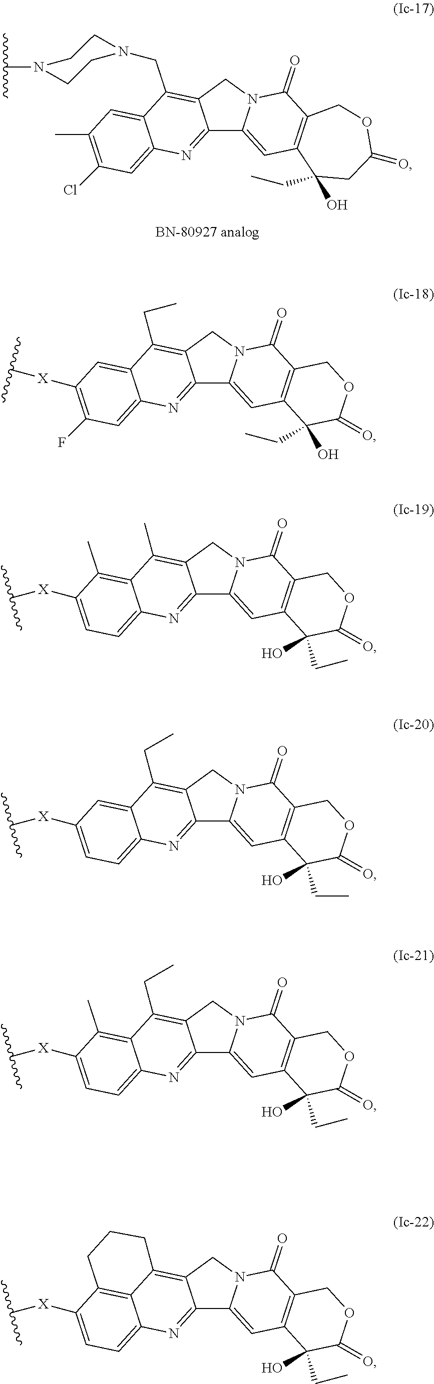

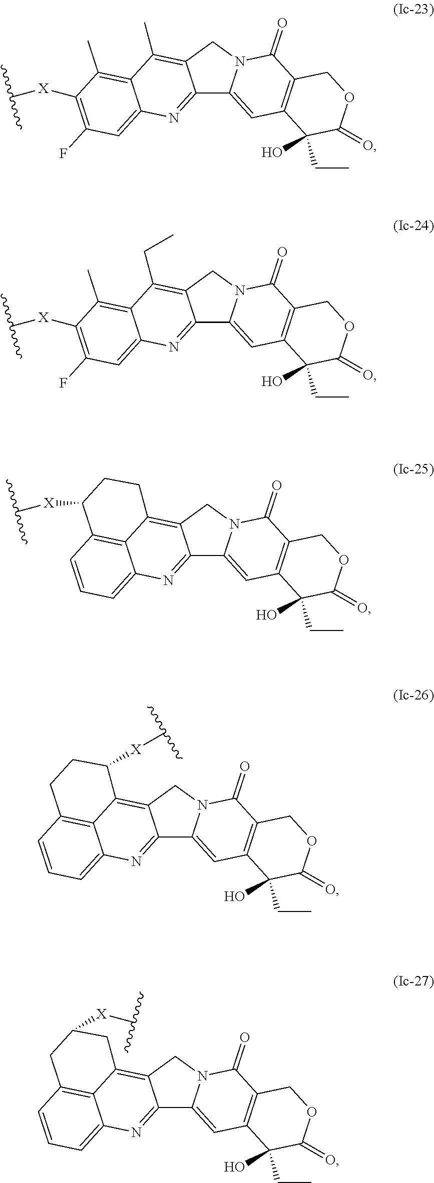

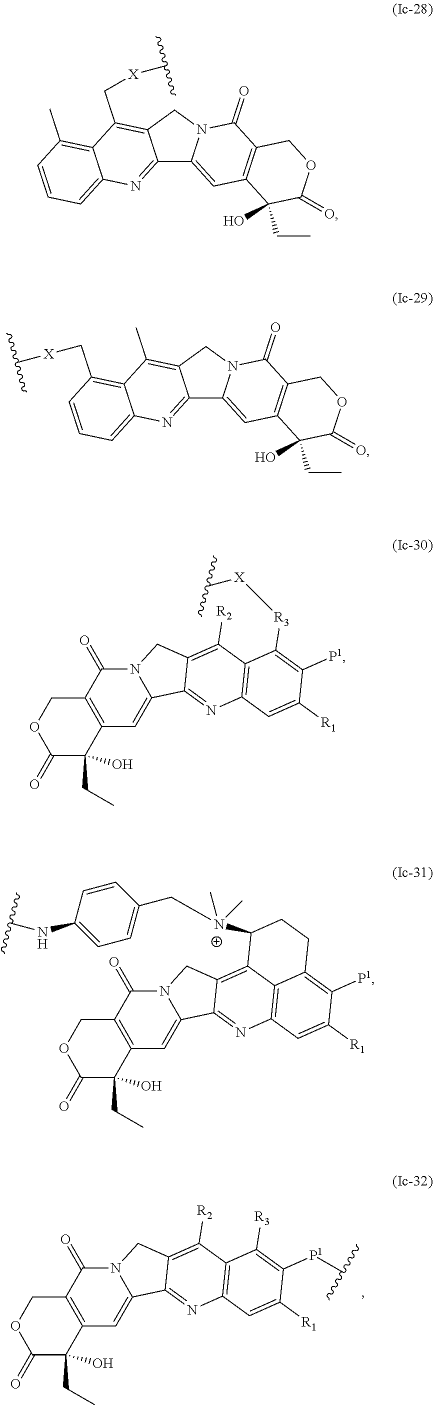

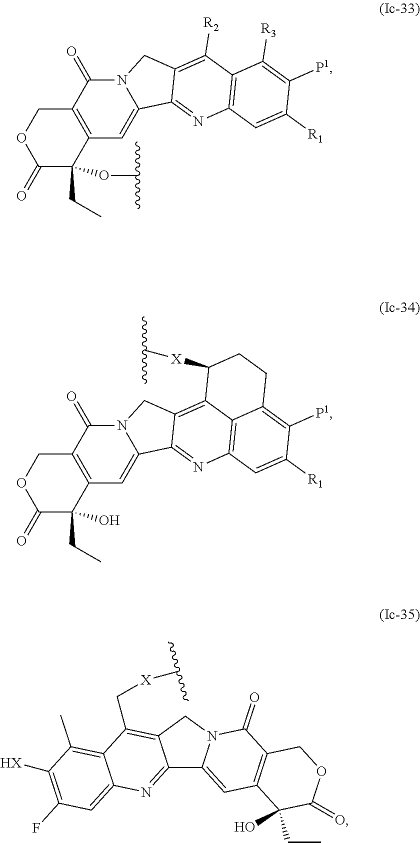

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] 0 is the site linked to L₁ or L₂; P¹ and R₁ are independently H, F, Cl, Br, I, SO₂R₂, SO₃H, CN, OH, NH₂, COOH, C(O)NH₂, O CH₂OP(O)(OR¹⁸)₂ , OC(O)OP(O)(OR¹⁸)₂ , OPO(OR¹⁸)₂, N HPO(OR¹⁸)₂ , OC(O)R¹⁸, OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, 0—(C₁-Cₛ of linear or branched alkyl), O—(C₁-C₈ of linear or branched alkyl)-OH, C₁-C₈ of linear or branched alkyl or heteroalkyl; C₂-Cₛ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR″ ?), carbamate (—C(O)NR⁷R¹⁸); R₂ and R₃ are independently H, C₁-C₈ of linear or branched alkyl, or C₂-C₈ of linear or branched heteroalkyl, ether, amine, ester, or amide; in addition, R₂ and R₃ can be joint together to form five or six member ring or cycloalkyl, cycloalkylamine, or cycloalkylamide, cyclohexylalkylamide; R¹⁷ and R¹⁸ are independently H, linear or branched alkyl or heteroalkyl; C₂-C₅ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R's); X is NH, O, S, S(02), NHS(O₂), NHS(O₂)NH, NHP(O)(OH), N+(R₂)(R₃), NHC(O)NH, NHC(O), NHC(O)O, N(CH₂CH₂)₂N, CON(CH₂CH₂)₂N, CON(CH₂CH₂)₂N+(R₂)(R₃), or CH₂.

In certain such embodiments, Combretastatins are natural phenols with vascular disruption properties in tumors. Exemplary combretastatins and their derivatives include, but are not limited to, combretastatin A-4 (CA-4), CA4-βGals, CA-4PD, CA4-NPs and ombrabulin, having the following formula:

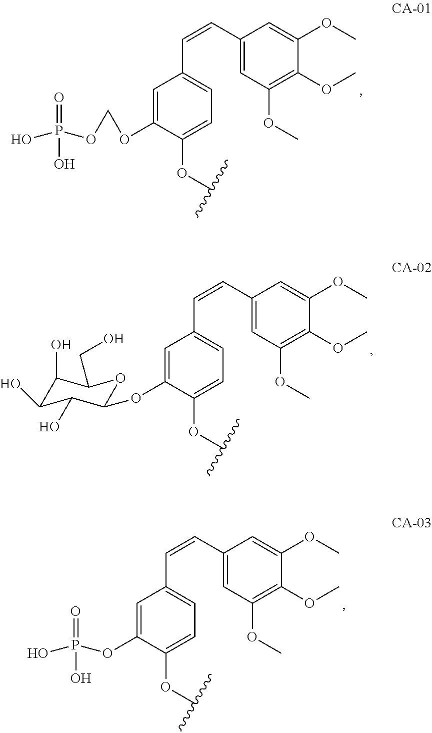 [SMILES: *Oc1ccc(/C=C\c2cc(OC)c(OC)c(OC)c2)cc1OCOP(=O)(O)O ; *Oc1ccc(/C=C\c2cc(OC)c(OC)c(OC)c2)cc1OC1OC(CO)C(O)C(O)C1O ; *Oc1ccc(/C=C\c2cc(OC)c(OC)c(OC)c2)cc1OP(=O)(O)O]

In certain such embodiments, Taxanes, which includes Paclitaxel (Taxol), a cytotoxic natural product, and docetaxel (Taxotere), a semi-synthetic derivative, and their analogs which are preferred for conjugation are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5 (2004); U.S. Patent No. 5, 475, 011 5, 728, 849, 5, 811, 452; 6, 340, 701; 6, 372, 738; 6, 391, 913, 6.436, 931; 6, 589, 979; 6, 596, 757; 6, 706, 708; 7, 008, 942; 7, 186, 851; 7, 217, 819; 7, 276, 499; 7, 598, 290; and 7, 667, 054. The structures of taxanes are preferred the following formula:

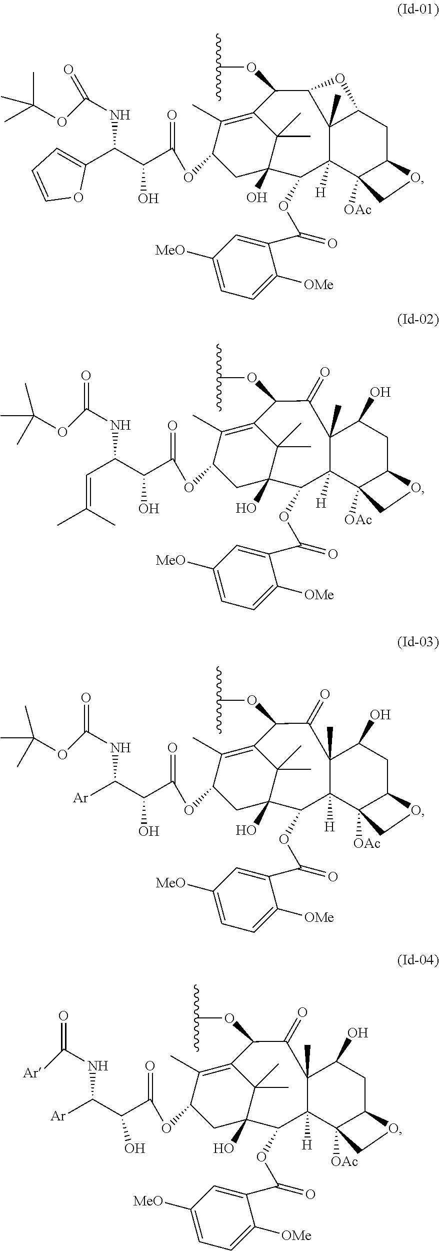

wherein [symbol] is the site linked to L₁ or L₂; Ar and Ar′ are independently aryl or heteroaryl.

In certain such embodiments, Anthracyclines are mammalian DNA topoisomerases II inhibitors that are able to stabilize enzyme-DNA complexes wherein DNA strands are cut and covalently linked to the antibody. These anticancer agents maintain a prominent role in treating many forms of solid tumors and acute leukemias during the last several decades. However, anthracyclines cause cardiovascular morbidity and mortality (Sagi, J. C., et al, Pharmacogenomics. 2016, 17(9), 1075-87; McGowan, J. V., et al, Cardiovasc Drugs Ther. 2017, 31(1), 63-75). Thus, to enhance specific activity of such molecules while reducing the cardiotoxicity, reasearchers actively are using the conjugation of anthracyclines to a cell-binding antibody, or antibody molecule as a general approach for improving the therapeutic index of these drugs, (Mollaev, M. et al, Int J Pharm. 2018 Dec 29. pii: S0378-5173(18) 30991-8; Rossin, R., et al, Bioconjug Chem. 2016, 27(7):1697-706; Dal Corso, A., et al, J Control Release. 2017, 264:211-218). Exemplary anthracyclines include, but are not limited to, daunorubicin, doxorubicin (i.e., adriamycin), epirubicin, idarubicin, valrubicin, and mitoxantrone. The structures of anthracyclines used for the present application are preferred the following formula:

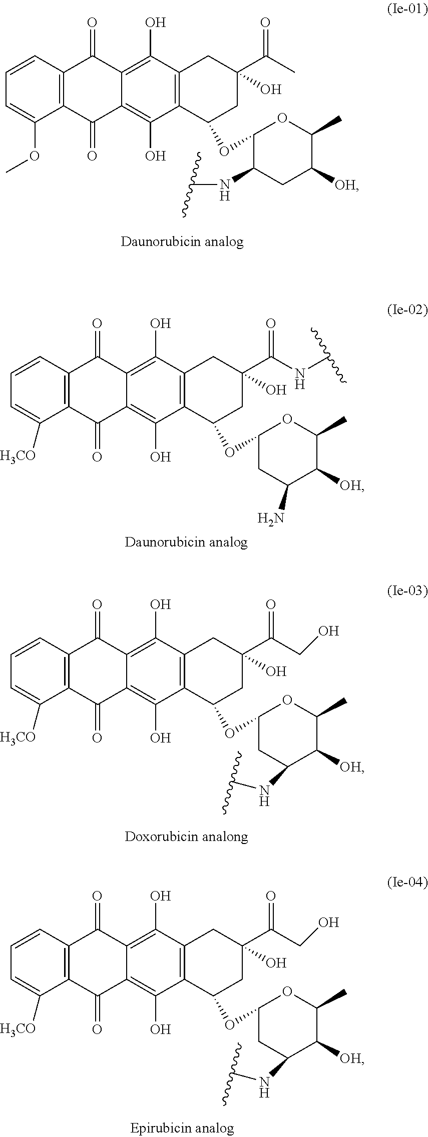

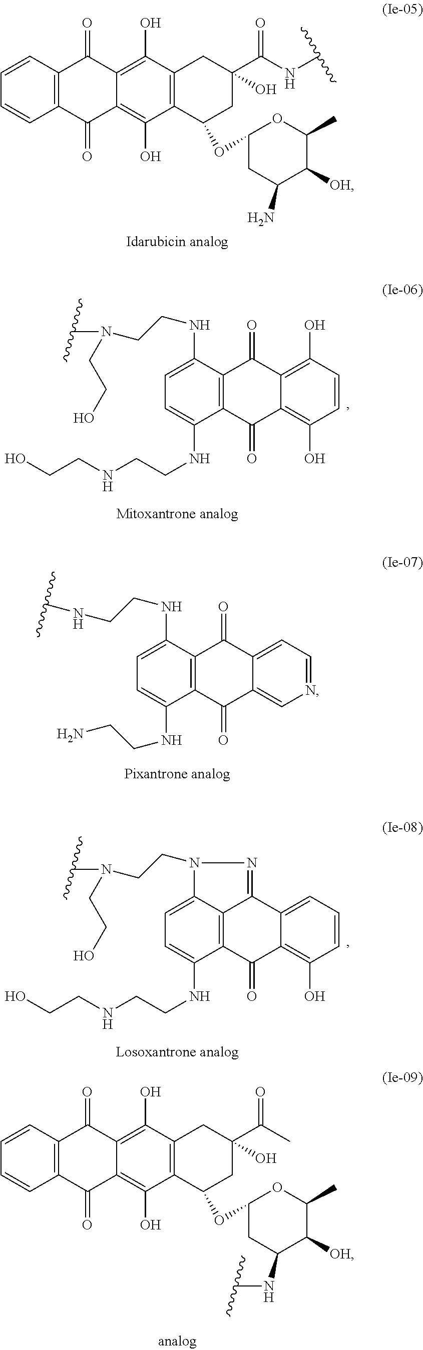

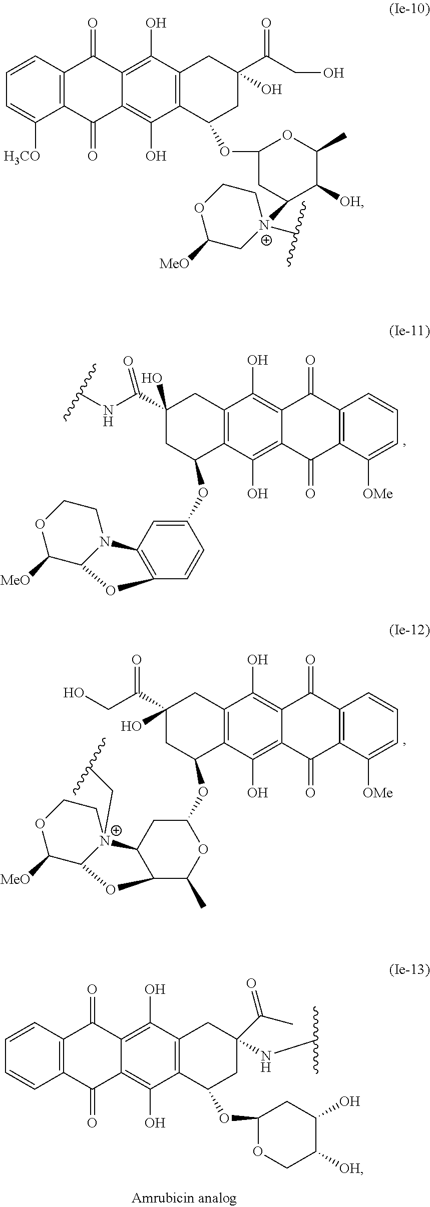

wherein [symbol] is the site that links to L₁ or L₂.

In certain such embodiments, Vinca alkaloids are a set of anti-mitotic and anti-microtubule alkaloid agents that work by inhibiting the ability of cancer cells to divide. Vinca alkaloids include vinblastine, vincristine, vindesine_leurosine, vinorelbine, catharanthine, vindoline, vincaminol, vineridine, minovincine, methoxyminovincine, minovincinine, vincadifformine, desoxyvincaminol, vincamajine, vincamine,vinpocetine,and vinburnine_The structures of vinca alkaloids are preferred vinblastine, vincristine having the following formula:

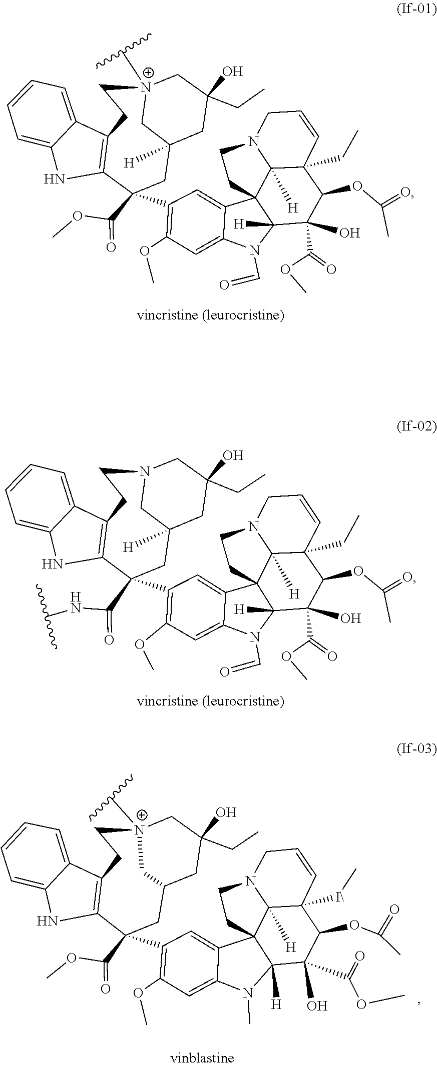

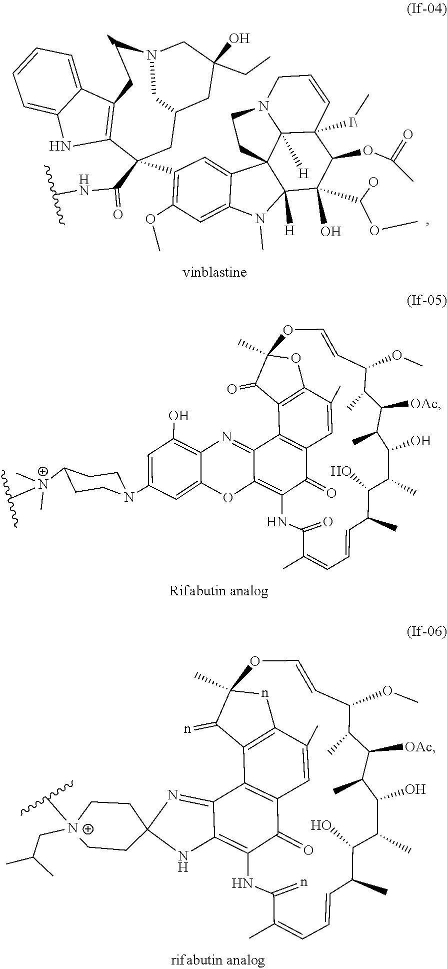

wherein [symbol] is the site linked to L₁ or L₂;

In certain such embodiments, Dolastatins and their peptidic analogs and derivatives, auristatins, are highly potent antimitotic agents that have been shown to have anticancer and antifungal activity. See, e.g., U.S. Pat. No. 5,663,149 and Pettit et al., Antimicrob. Agents Chemother. 42:2961-2965, 1998. Exemplary dolastatins and auristatins include, but are not limited to, dolastatin 10, auristatin E (AE), auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproimenolastatine), 5-benzoylvaleric acid-AE ester (AEVB), Auristatin F phenylene diamine (AFP) and other novel auristatins. The auristatins are described in Int. J. Oncol. 15: 367-72 (1999); Molecular Cancer Therapeutics, vol. 3, No. 8, pp. 921-32 (2004); U.S. Application Nos. 11134826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. The structures of auristatin analogs are preferred the following formula (Ih-01), (Ih-02), (Ih-03), (Ih-04), (Ih-05), (Ih-06), (Ih-07), (Ih-08), (Ih-09), (Ih-10), and (Ih-11):

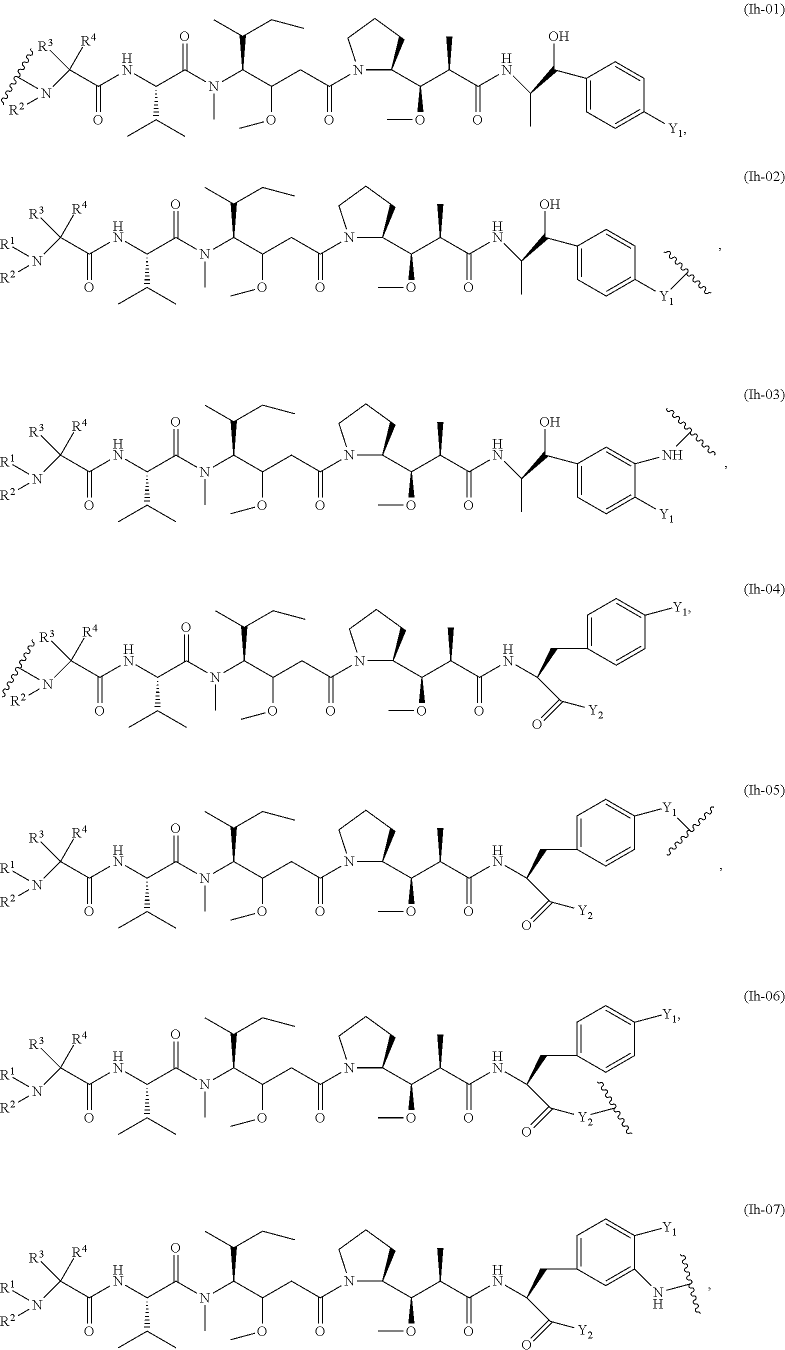

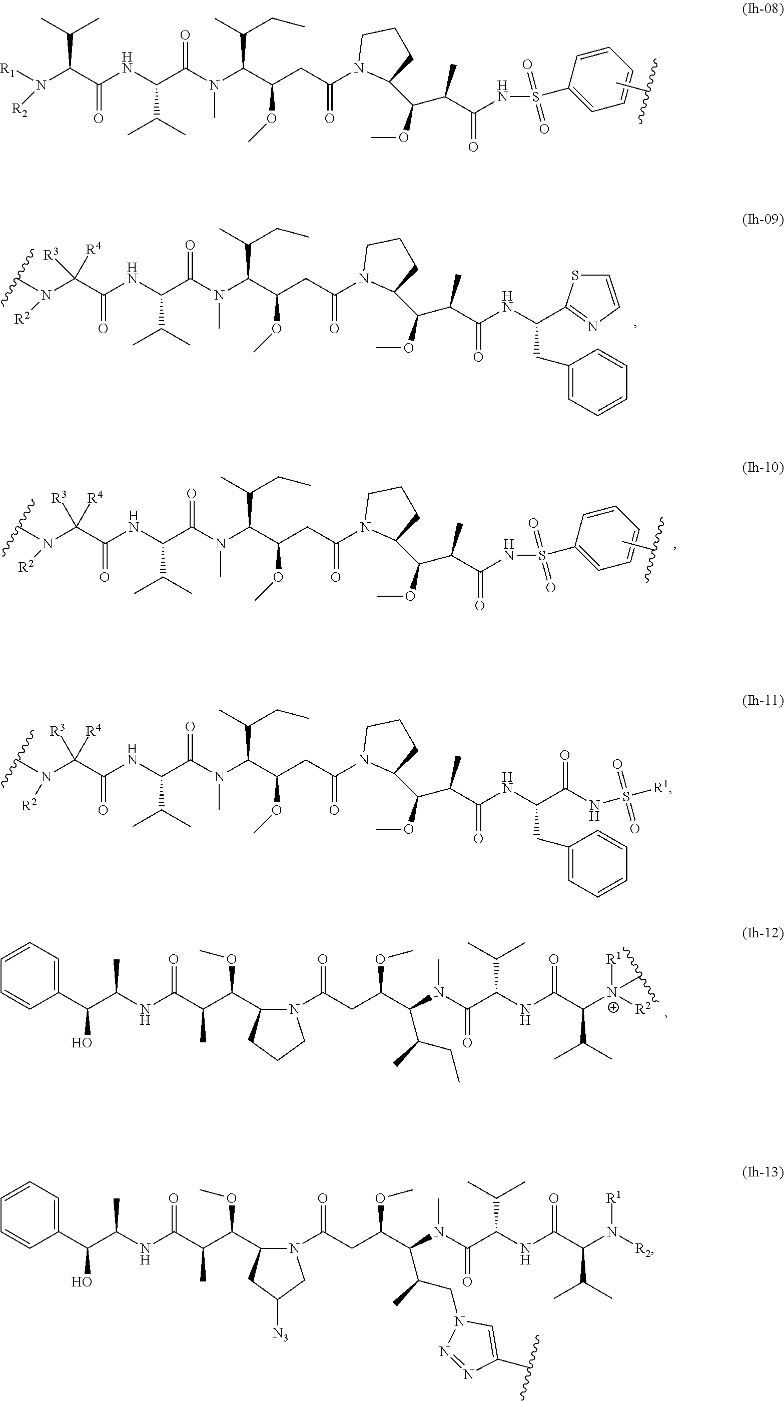

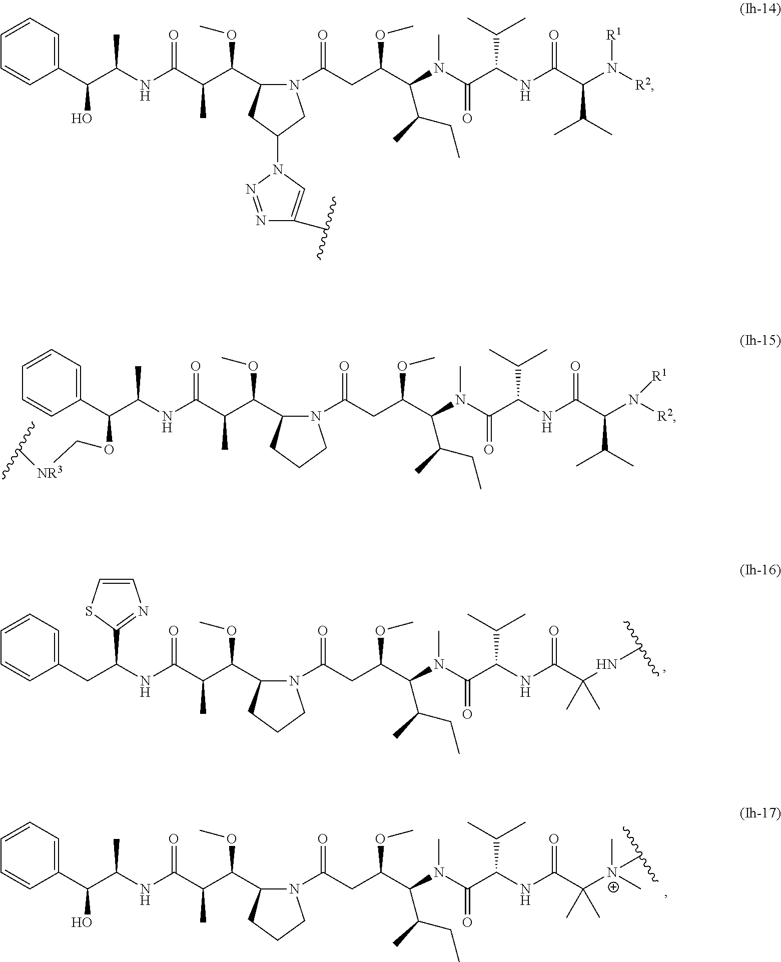

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 1000. The two Rs: R′R², R²R₃, R′R³ or R³R⁴ together can form 3~8 member cyclic ring of alkyl, aryl, heteroaryl, heteroalkyl, or alkylcycloalkyl group; Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR¹ when linked to the connecting site “ [symbol] ” (that links to L₁ and/or L₂ independently); or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; R₁₂ is OH, NH₂, NHR¹, NHNH₂, NHNHCOOH, O—RI—COOH, NH—RI—COOH, NH—(Aa)COOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-500; R₁, R₂, R₃, R₄, R₅, R₅′, Z₁, Z₂, and n are defined the same above.

In certain such embodiments, Hemiasterlin and its analogues (e.g., HTI-286) bind to the tubulin, disrupt normal microtubule dynamics, and, at stoichiometric amounts, depolymerize microtubules. The structures of hemiasterlins are preferred the following formula:

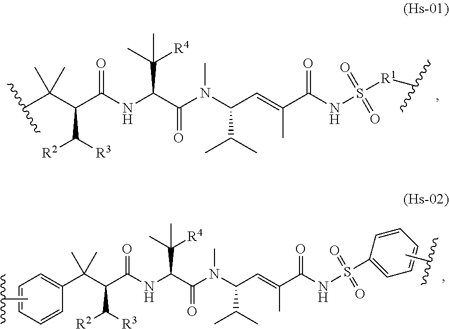

wherein wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; In addition, R²R₃ can form 3-8 member cyclic ring of alkyl, aryl, heteroaryl, heteroalkyl, or alkylcycloalkyl group.

In certain such embodiments, Eribulin which is binding predominantly to a small number of high affinity sites at the plus ends of existing microtubules has both cytotoxic and non-cytotoxic mechanisms of action. Its cytotoxic effects are related to its antimitotic activities, wherein apoptosis of cancer cells is induced following prolonged and irreversible mitotic blockade (Kuznetsov, G. et al, Cancer Research. 2004, 64 (16): 5760-6.; Towle, M. J, et al, Cancer Research. 2010, 71 (2): 496-505). In addition to its cytotoxic, antimitotic-based mechanisms, preclinical studies in human breast cancer models have shown that eribulin also exerts complex effects on the biology of surviving cancer cells and residual tumors that appear unrelated to its antimitotic effects. Eribulin has been approved by US FDA for the treatment of metastatic breast cancer who have received at least two prior chemotherapy regimens for late-stage disease, including both anthracycline- and taxane-based chemotherapies, as well as for the treatment of liposarcoma (a specific type of soft tissue sarcoma) that cannot be removed by surgery (unresectable) or is advanced (metastatic). Eribulin has been used as payload for ADC conjugates (US20170252458). The structure of Eribulin is preferred the following formula, Eb01:

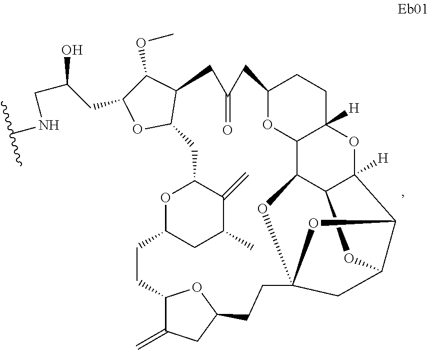 [SMILES: *NC[C@@H](O)C[C@H]1O[C@H]2C[C@H]3O[C@@H](CC[C@@H]4O[C@@H](CC[C@@]56C[C@H]7O[C@@H]8[C@@H](O[C@H]9CC[C@H](CC(=O)C[C@@H]2[C@H]1OC)OC9[C@@H]8O5)[C@H]7O6)CC4=C)C[C@@H](C)C3=C]

[symbol]

is a linkage site that links to L₁ and/or L₂ independently;

In certain such embodiments, an Inhibitor of nicotinamide phosphoribosyltransferases (NAMPT) can be an interesting ADC payload due to their unique mechanisms of high potent activity (Sampath D, et al, Pharmacol Ther 2015; 151, 16-31). NAMPT regulates nicotinamide adenine dinucleotide (NAD) levels in cells wherein NAD plays as an essential redox cofactor to support energy and anabolic metabolism. NAD has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that can be released from cells spontaneously and by regulated mechanisms (Smyth L. M, et al, J. Biol. Chem. 2004, 279 (47), 48893-903; Billington R. A, et al, Mol Med. 2006, 12, 324-7), and can therefore have important extracellular roles (Billington R. A, et al, Mol Med. 2006, 12, 324-7). When inhibitors of NAMPT present, NAD levels decline below the level needed for metabolism resulting in energy crisis and therefore cell death. So far, clinical NAMPT inhibitor candidates FK-866, CHS-828, and GMX-1777 advanced to clinical trials but each encountered dose-limiting toxicities prior to any objective responses (Holen K., et al, Invest New Drugs 2008, 26, 45-51; Hovstadius, P., et al, Clin Cancer Res 2002, 8, 2843-50; Pishvaian, M. J., et al, J Clin Oncol 2009, 27, 3581). Thus, using ADCs for targeting delivery of NAMPT inhibitors might circumvent the systemic toxicities to achieve much broader therapeutic index. The structures of NAMPT inhibitors are preferred the following formula, NPO1, NPO2, NP03, NPO4, NP05, NP06, NPO₇, NP08, and NPO9:

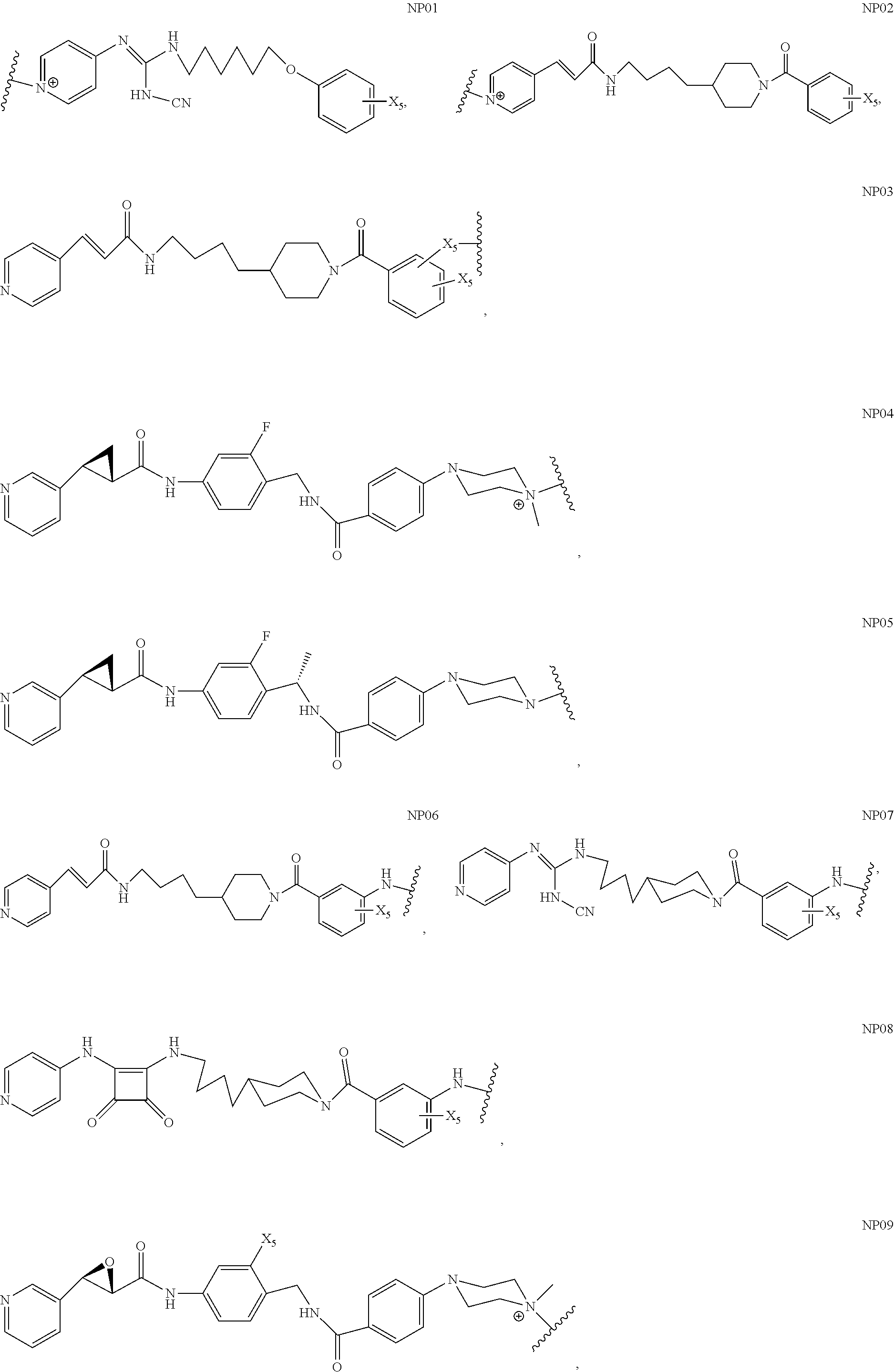

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR₁, OCOR₁, NHCOR₁.

In certain such embodiments, a benzodiazepine dimer and its analogs: (e. g. a dimer of pyrrolobenzodiazepine (PBD) or (tomaymycin), a dimer of indolinobenzodiazepine (IGN), a dimer of imidazobenzothiadiazepine, or a dimer of oxazolidinobenzodiazepines) are anti-tumor agents that contain one or more imine functional groups, or their equivalents, that bind to duplex DNA. PBD and IGN molecules are based on the natural product anthramycin, and interact with DNA in a sequence-selective maimer, with a preference for purine-guanine-purine sequences. The preferred benzodiazepine dimers according to the present invention are exampled in: US Patent Nos. 8, 163, 736; 8, 153, 627; 8, 034, 808; 7, 834, 005; 7, 741, 319; 7, 704, 924; 7, 691, 848; 7, 678, 787; 7, 612, 062; 7, 608, 615; 7, 557, 099; 7, 528, 128; 7, 528, 126; 7, 511, 032; 7, 429, 658; 7, 407, 951; 7, 326, 700; 7, 312, 210; 7, 265, 105; 7, 202, 239; 7, 189, 710; 7, 173, 026; 7, 109, 193; 7, 067, 511; 7, 064, 120; 7, 056, 913; 7, 049, 311; 7, 022, 699; 7, 015, 215; 6, 979, 684; 6, 951, 853; 6, 884, 799; 6, 800, 622; 6, 747, 144; 6, 660, 856; 6, 608, 192; 6, 562, 806; 6, 977, 254; 6, 951, 853; 6, 909, 006; 6, 344, 451; 5, 880, 122; 4, 935, 362; 4, 764, 616; 4, 761, 412; 4, 723, 007; 4, 723, 003; 4, 683, 230; 4, 663, 453; 4, 508, 647; 4, 464, 467; 4, 427, 587; 4, 000, 304; US patent appl. 20100203007, 20100316656, 20030195196. Examples of the structures of the conjugate of the antibody- benzodiazepine dimers are illustrated below PBO1 ~PB30:

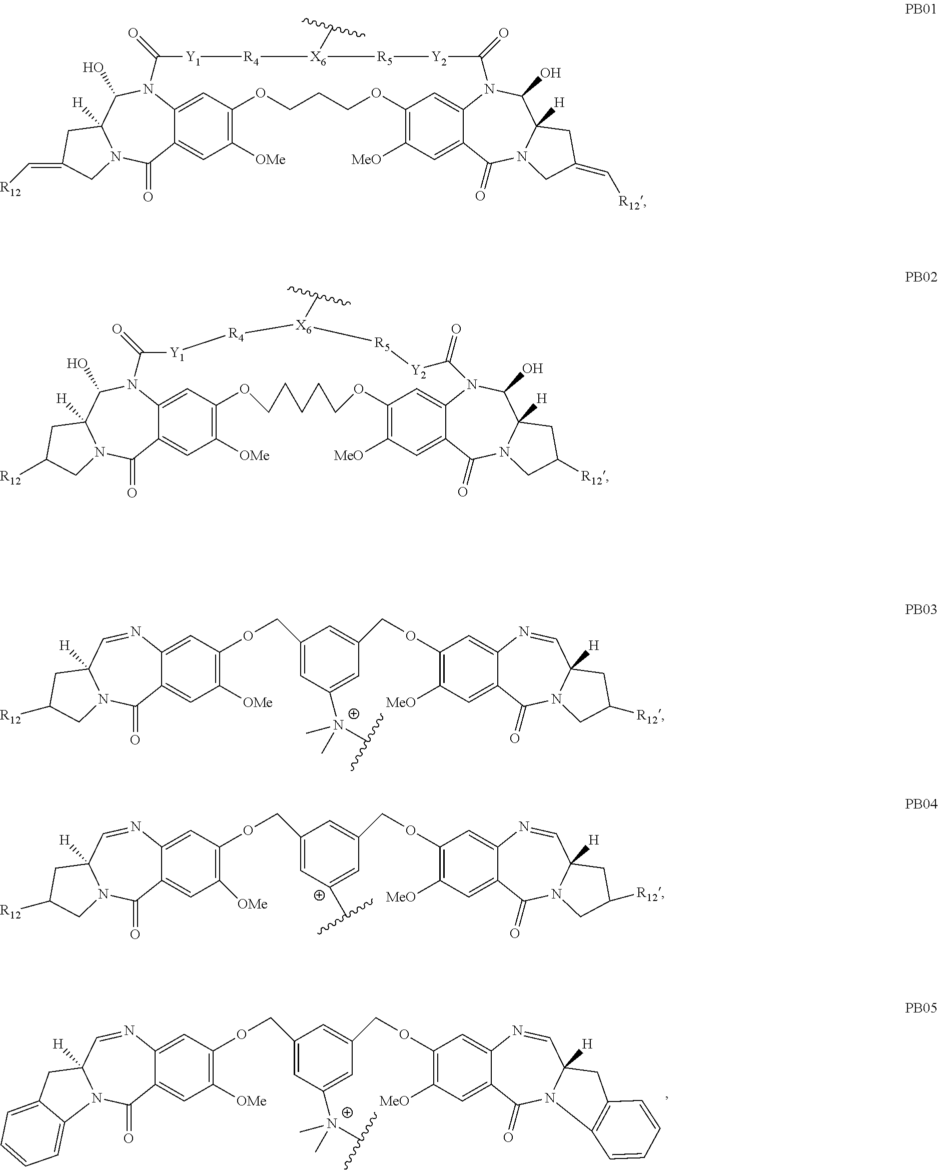

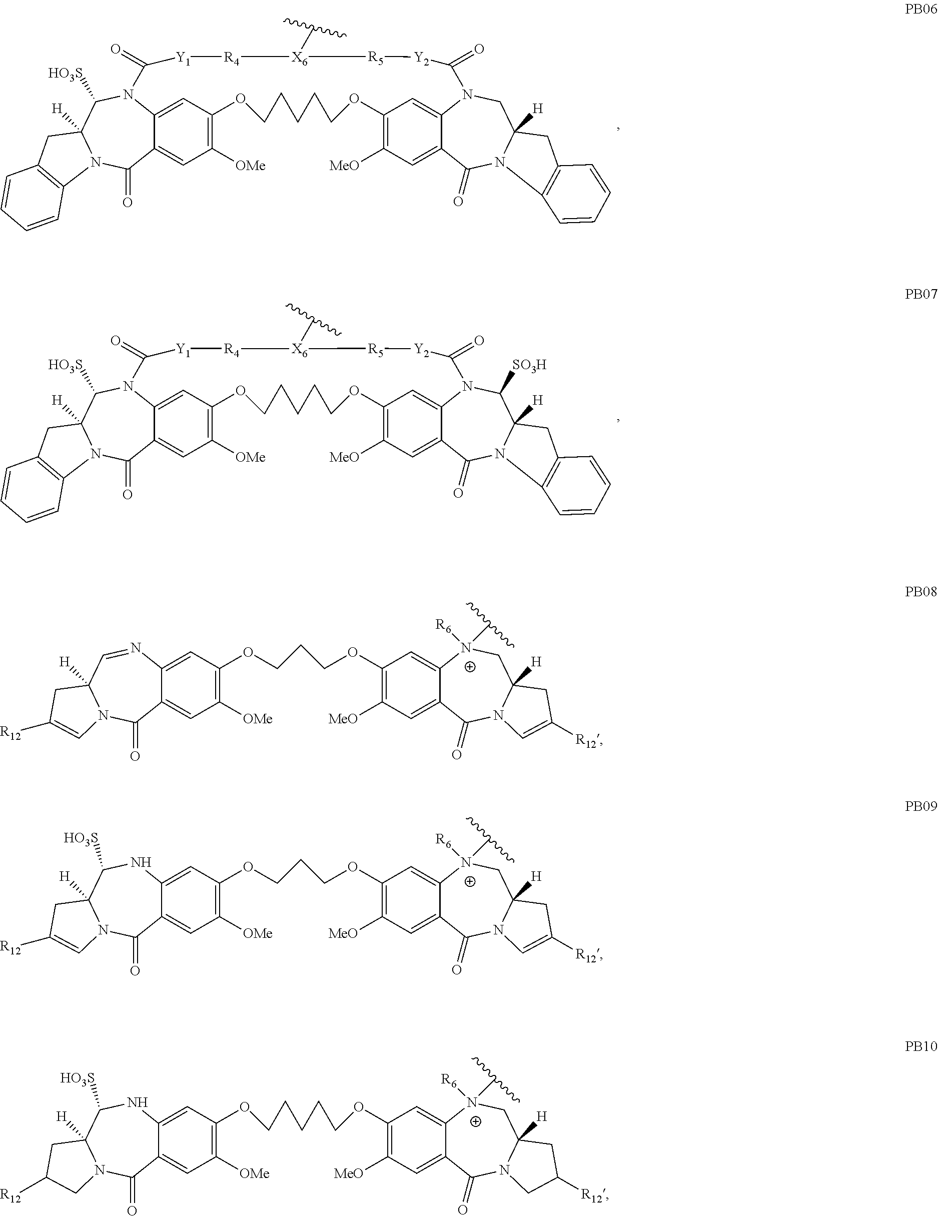

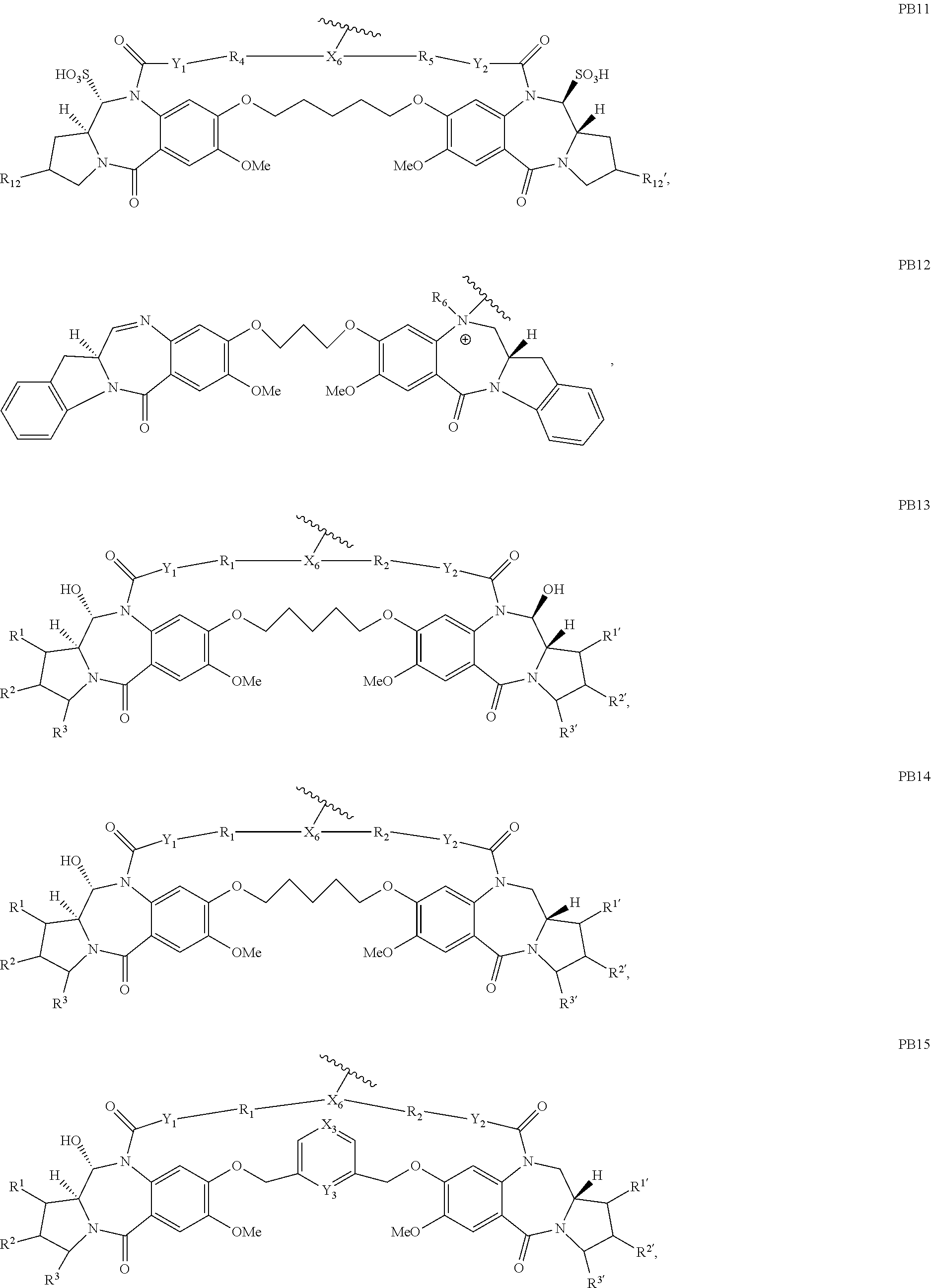

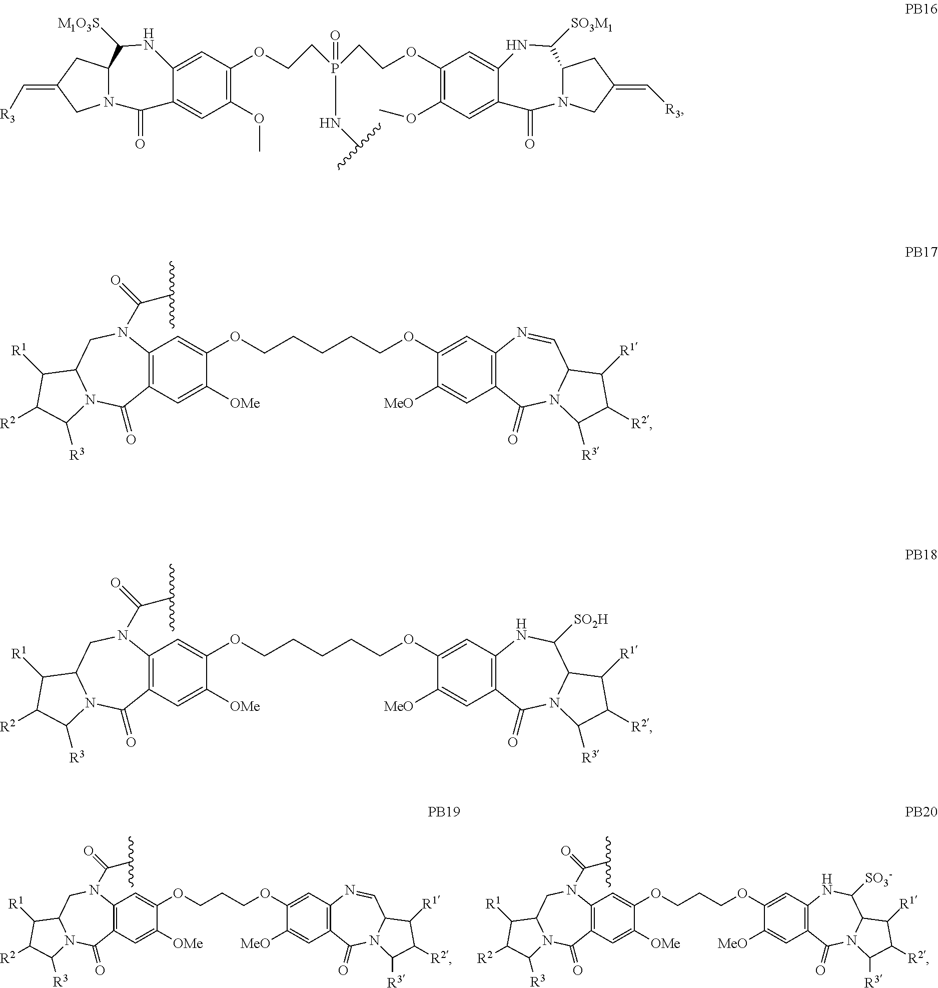

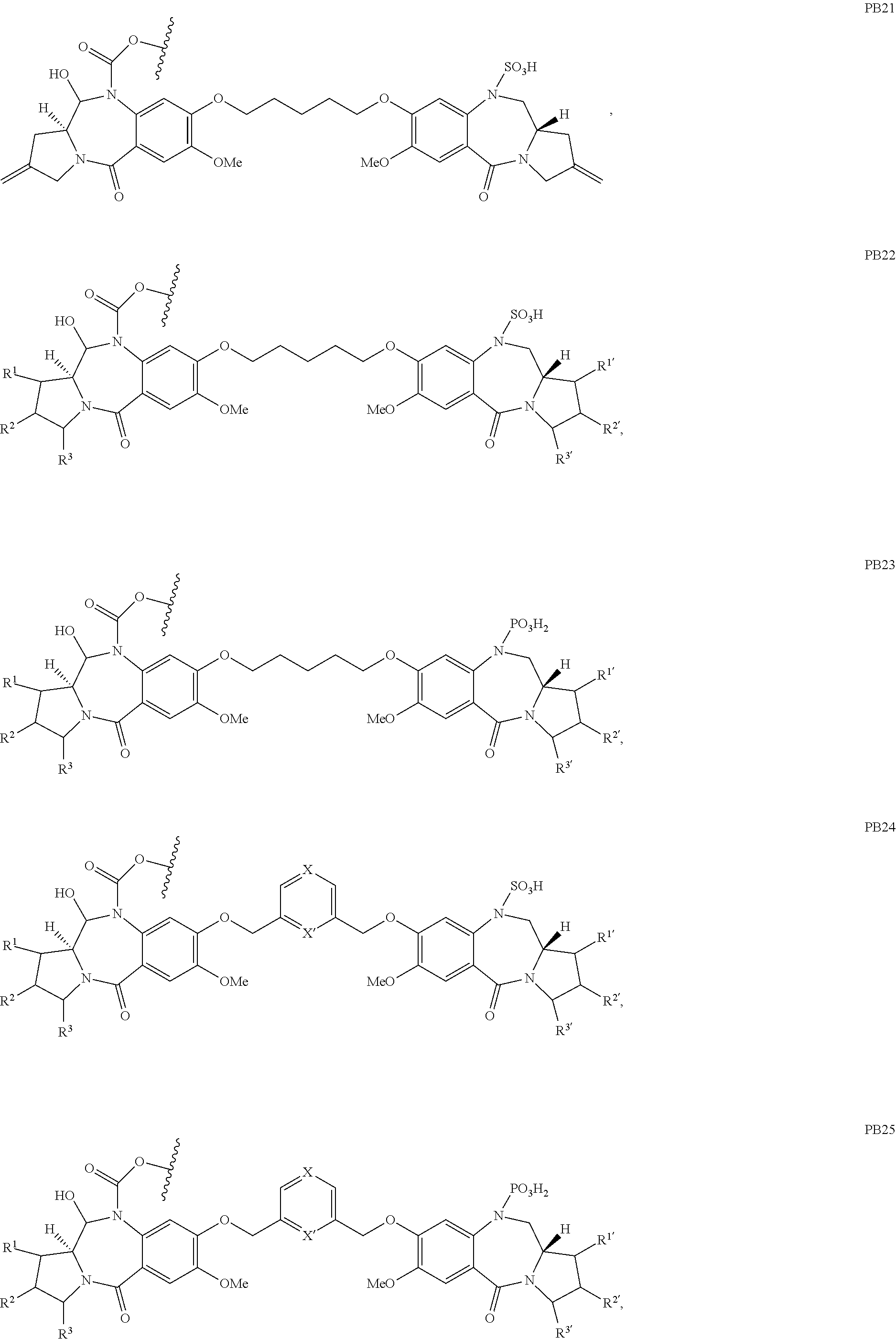

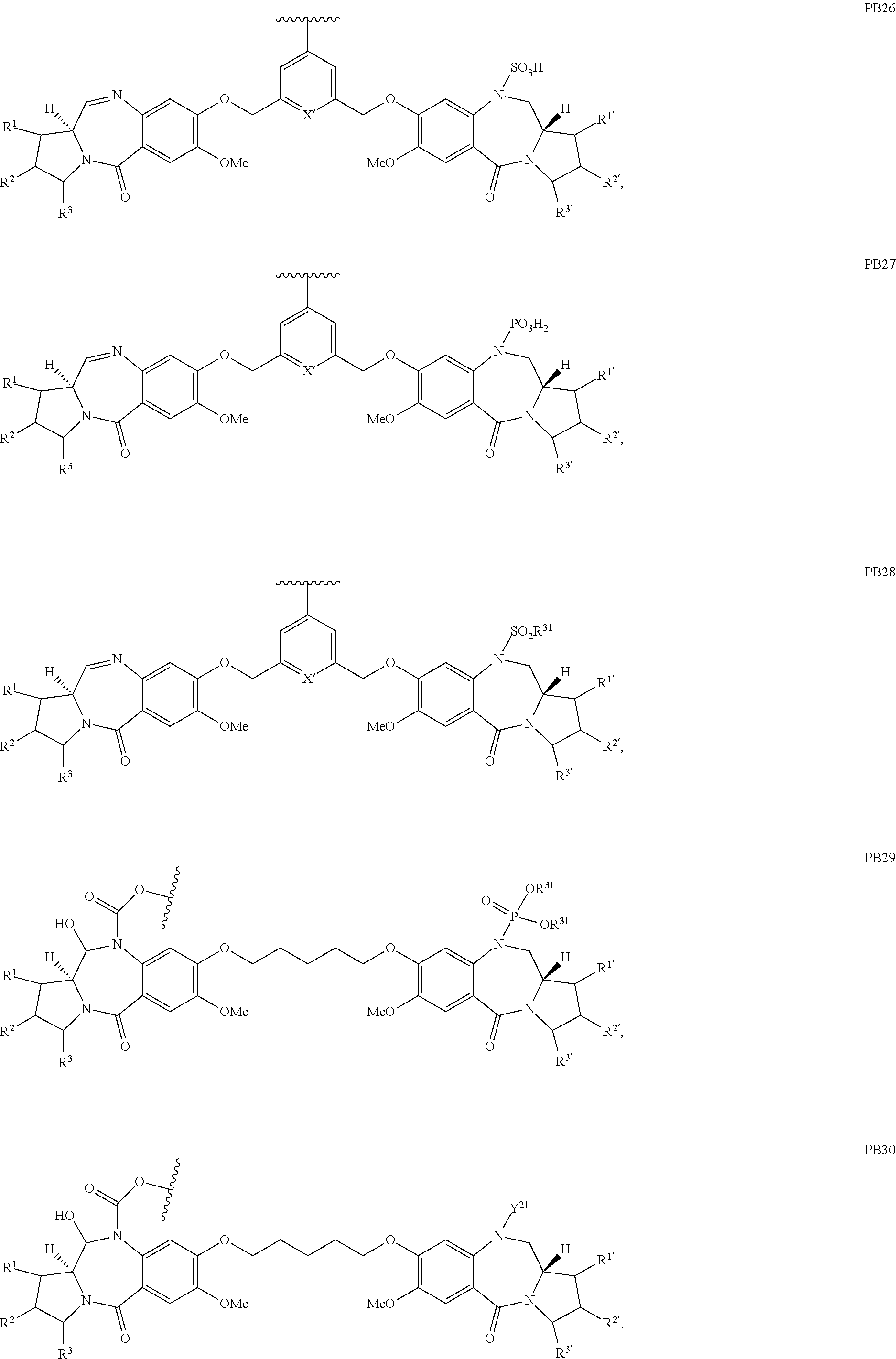

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁, Y₂, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R¹, R², R³, R^{4′}, R^{2′}, and R^{3′} are independently H; F; Cl; ═O; ═S; —CH₂; =CH—R₁, OH; SH; C₁-C₈ linear or branched alkyl, aryl, alkenyl, heteroaryl, heteroalkyl, alkylcycloalkyl, ester (COOR₅ or —OC(O)R₈), ether (OR₅), amide (CONR₅), carbamate (OCONR₅), amines (NHR₅, NR₅R₅′), heterocycloalkyl, or acyloxylamines (—C(O)NHOH, —ONHC(O)R₅); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000. The two Rs: R¹R², R²R₃, R^{1′}, R^{2′}, or R^{2′}R₃′ can independently form 3-8-member cyclic ring of alkyl, aryl, heteroaryl, heteroalkyl, or alkylcycloalkyl group;
X₃ and Y₃ are independently N, NH, CH₂ or CR₅, or one of X₃ and Y₃ can be absent;
wherein R₁, and R₂ are C₁-C₈ linear or branched alkyl, heteroalkyl; C₃-C₈ aryl, heteroaryl, alkylcycloalkyl, acyloxyl, alkylaryl, alkylaryloxyl, alkylarylamino, alkylarylthiol; or 1-6 the same or different sequence of amino acid/peptides (Ar)r, r=1-6;
wherein R₄, R₅, R₅′, R₆, R₁₂ and R₁₂′ are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, acyloxylamines;
Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃;
X₆ is CH, N, P(O)NH, P(O)NR₁, CHC(O)NH, C₃-C₈ aryl, heteroaryl, alkylcycloalkyl, acyloxyl, alkylaryl, alkylaryloxyl, alkylarylamino, or an Aa (amino acid, is preferably selected from Lys, Phe, Asp, Glu, Ser, Thr, His, Cys, Tyr, Trp, Gln, Asn, Arg);
X and X′ are independently CH₂, or N, and X and/or X′ can be O, S or NH when the six-member aromatic ring that they involved become five-member ring;
Y^{2L} is Ms (mesyl), Ts (tosyl) or Tf (triflyl), SO₃H, P(O)(OH)₂, CH₂(O)P(O)(OH)₂, glycoside;
R³¹ is H, C1 —C8 alkyl or Ar, CF₃; “ [symbol] ” is defined the same above.

In certain such embodiments, a CC-1065 analog and duocarmycin analogs are also preferred to be used for a conjugate of the present process invention. The examples of the CC-1065 analogues and duocarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5786486, 5789650, 5814318, 5846545, 5874299, 5877296, 5877397, 5885793, 5939598, 5962216, 5969108, 5985908, 6060608, 6066742, 6075181, 6103236, 6114598, 6130237, 6132722, 6143901, 6150584, 6162963, 6172197, 6180370, 6194612, 6214345, 6262271, 6281354, 6310209, 6329497, 6342480, 6486326, 6512101, 6521404, 6534660, 6544731, 6548530, 6555313, 6555693,6566336,6, 586, 618, 6593081, 6630579, 6, 756, 397, 6759509, 6762179, 6884869, 6897034, 6946455, 7, 049, 316, 7087600, 7091186, 7115573, 7129261, 7214663, 7223837, 7304032, 7329507, 7, 329, 760, 7, 388, 026, 7, 655, 660, 7, 655, 661, 7, 906, 545, and 8, 012, 978. Examples of the structures of the conjugate of the antibody-CC-1065 analogs via the linker of the patent are illustrated below CCO₁, CC02, CC03, CC04, CC05, CC06 and CC07:

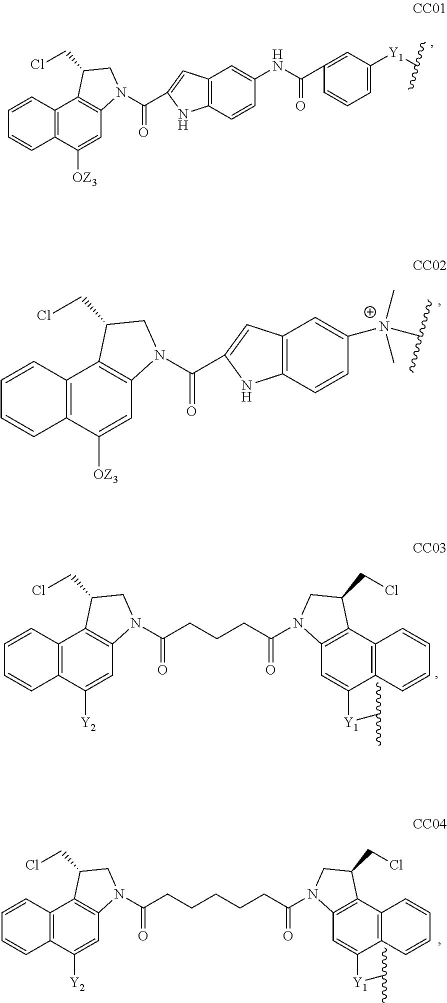

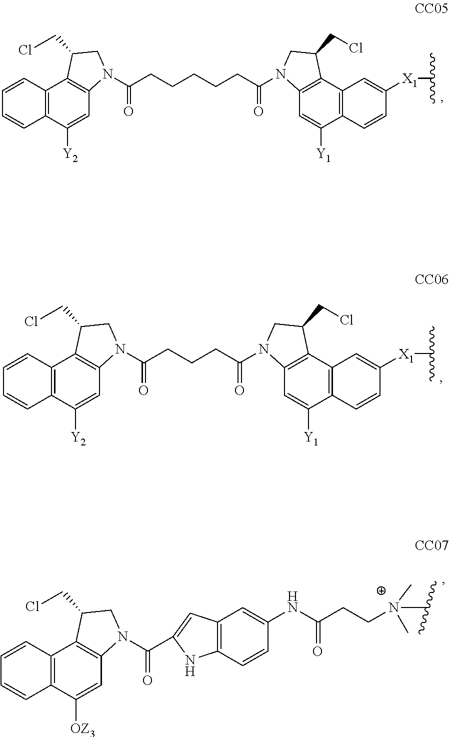

wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are defined the same above; In certain such embodiments, an amatoxin and its analogs which are a subgroup of at least ten toxic compounds originally found in several genera of poisonous mushrooms, most notably Amanita phalloides and several other mushroom species, are also preferred for conjugation of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American232 (3): 90-101; H. E. Hallen, et al 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15(16): 3480-4; Fiume, L., Curr Probl Clin Biochem, 1977, 7: 23-8; Karlson-Stiber C, Persson H. 2003, Toxicon 42(4): 339-49; Chafin, D. R., Guo, H. & Price, D. H. 1995 J. Biol. Chem. 270 (32): 19114-19; Wieland (1983) Int. J. Pept. Protein Res. 22(3): 257-76.). Amatoxins can be produced from collected Amanita phalloides mushrooms (Yocum, R. R. 1978 Biochemistry 17(18): 3786-9; Zhang, P. et al, 2005, FEMS Microbiol. Lett.252(2), 223-8), or from fermentation using a basidiomycete (Muraoka, S. and Shinozawa T., 2000 J. Biosci. Bioeng. 89(1): 73-6) or from fermentation using A. fissa (Guo, X. W., et al, 2006 Wei Sheng Wu Xue Bao 46(3): 373-8), or from culturing Galerina fasciculata or Galerina helvoliceps, a strain belonging to the genus (WO/1990/009799, JP11137291). However, the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18(2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870(3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32(1): 9-20; G. Zanotti, T. et al, Int. J. Peptide Protein Res. 1989, 34, 222-8; Zanotti, G., et al., Int J Pept Protein Res, 1990. 35(3): 263-70; Mullersman, J. E. and J. F. Preston, 3rd, Int J Pept Protein Res, 1991. 37(6): 544-51; Mullersman, J. E., et al, Int J Pept Protein Res, 1991. 38(5): 409-16; Zanotti, G., et al, Int J Pept Protein Res, 1992. 40(6): 551-8; Schmitt, W. et al, J. Am. Chem. Soc. 1996, 118, 4380-7; Anderson, M.O., et al, J. Org. Chem., 2005, 70(12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88(5): 714-24; May, J. P., et al., Chemistry, 2008. 14(11): 3410-7; S. De Lamo Marin, et al, Eur. J. Org. Chem. 2010, 3985-9; Pousse, G., et al., Org Lett, 2010. 12(16): 3582-5; Luo, H., et al., Chem Biol, 2014. 21(12): 1610-7; Zhao, L., et al., Chembiochem, 2015. 16(10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243(130): 281-3; Bonetti, E., M. et al, Arch Toxicol, 1976. 35(1): p. 69-73; Davis, M. T., Preston, J. F. Science 1981, 213, 1385-1388; Preston, J. F., et al, Arch Biochem Biophys, 1981. 209(1): 63-71; H. Faulstich, et al, Biochemistry 1981, 20, 6498-504; Barak, L. S., et al., Proc Natl Acad Sci USA, 1981. 78(5): 3034-8; Faulstich, H. and L. Fiume, Methods Enzymol, 1985. 112: 225-37; Zhelev, Z., A. et al, Toxicon, 1987. 25(9): 981-7; Khalacheva, K., et al, Eksp Med Morfol, 1990. 29(3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J.E. and J. F. Preston, Biochem Cell Biol, 1991. 69(7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16(10): 1420-5; Zhou, B., et al., Biosens Bioelectron, 2015. 68:189-96; WO2014/043403, US20150218220, EP 1661584). We have been working on the conjugation of amatoxins for a while. Examples of the structures of the amatoxins used for the present application are preferred the following structures of Am01, Am02, and Am03:

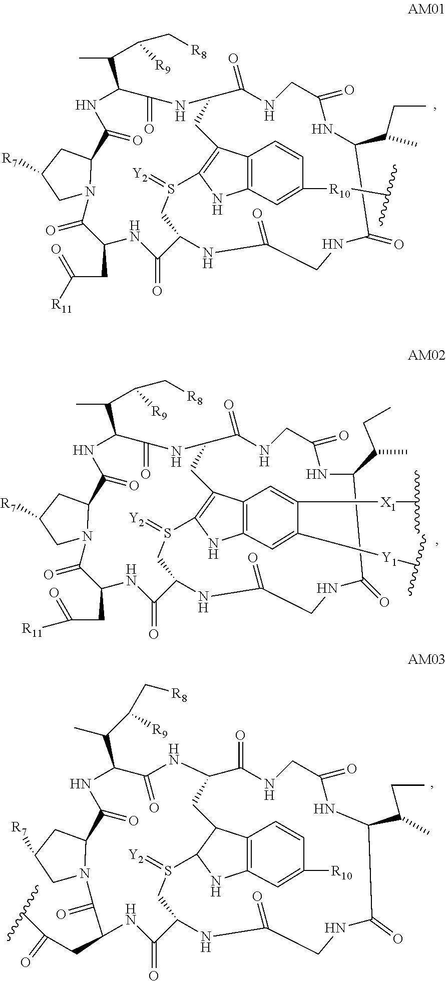

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NRs, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NHNHC(O) and C(O)NR; R₇, Rs, and R₉ are independently H, OH, OR₁, NH₂, NHR₁, C₁-C₆ alkyl, or absent; Y₂ is 0, 02, NR₁, NH, or absent; Rio is CH₂, O, NH, NR₁,NHC(O), NHC(O)NH, NHC(O)O, OC(O)O, C(O), OC(O), OC(O)(NR), (NR₁)C(O)(NR₁), C(O)R₁ or absent; R₁₁ is OH, NH₂, NHR, NHNH₂, NHNHCOOH, O—R, —COOH, NH—R₁—COOH, NH—(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR¹, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa), is 1-8 aminoacids; n and mi are independently 1-20; p is 1-500; R₁, R₂ and Ar, are the same defined throughout the application; “ [symbol] ” is defined the same above.

In certain such embodiments, spliceostatins and pladienolides are anti-tumor compounds which inhibit splicing and interacts with spliceosome, SF3b. Examples of spliceostatins include, but are not limited to, spliceostatin A, FR901464, and (2S, 3Z)-5-{[(2R, 3R, 5S, 6S)-6-{(2E, 4E)-5-[(3R, 4R, 5R, 7S)-7-(2-hydrazinyl-2-oxoethyl)-4-hydroxy-1, 6-dioxaspiro[2.5]oct-5-yl]-3-methylpenta-2, 4-dien-1-y-l}-2, 5-dimethyltetrahydro-2H-pyran-3-yl]amino}-5-oxopent-3-en-2-yl acetate having the core structure:

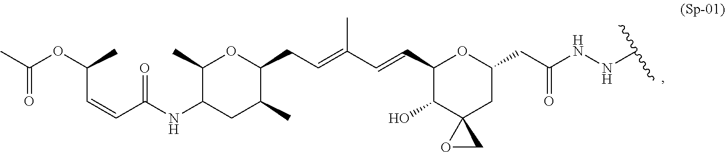 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1]

Examples of pladienolides include, but are not limited to, Pladienolide B, Pladienolide D, and E7107.

In certain such embodiments, protein kinase inhibitors that block the action of an enzyme to add a phosphate (PO₄) group to serine, threonine, or tyrosine amino acids on an antibody, and can modulate the protein function. The protein kinase inhibitors can be used to treat diseases due to hyperactive protein kinases (including mutant or overexpressed kinases) in cancer or to modulate cell functions to overcome other disease drivers. The structures of protein kinase inhibitors are preferred to selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, Vemurafenib, Entrectinib, Palbociclib, Ribociclib, Abemaciclib, Dacomitinib, Neratinib, Rociletinib (CO-1686), Osimertinib, AZD3759, Nazartinib (EGF816), having the following formula, PKO1 PK40:

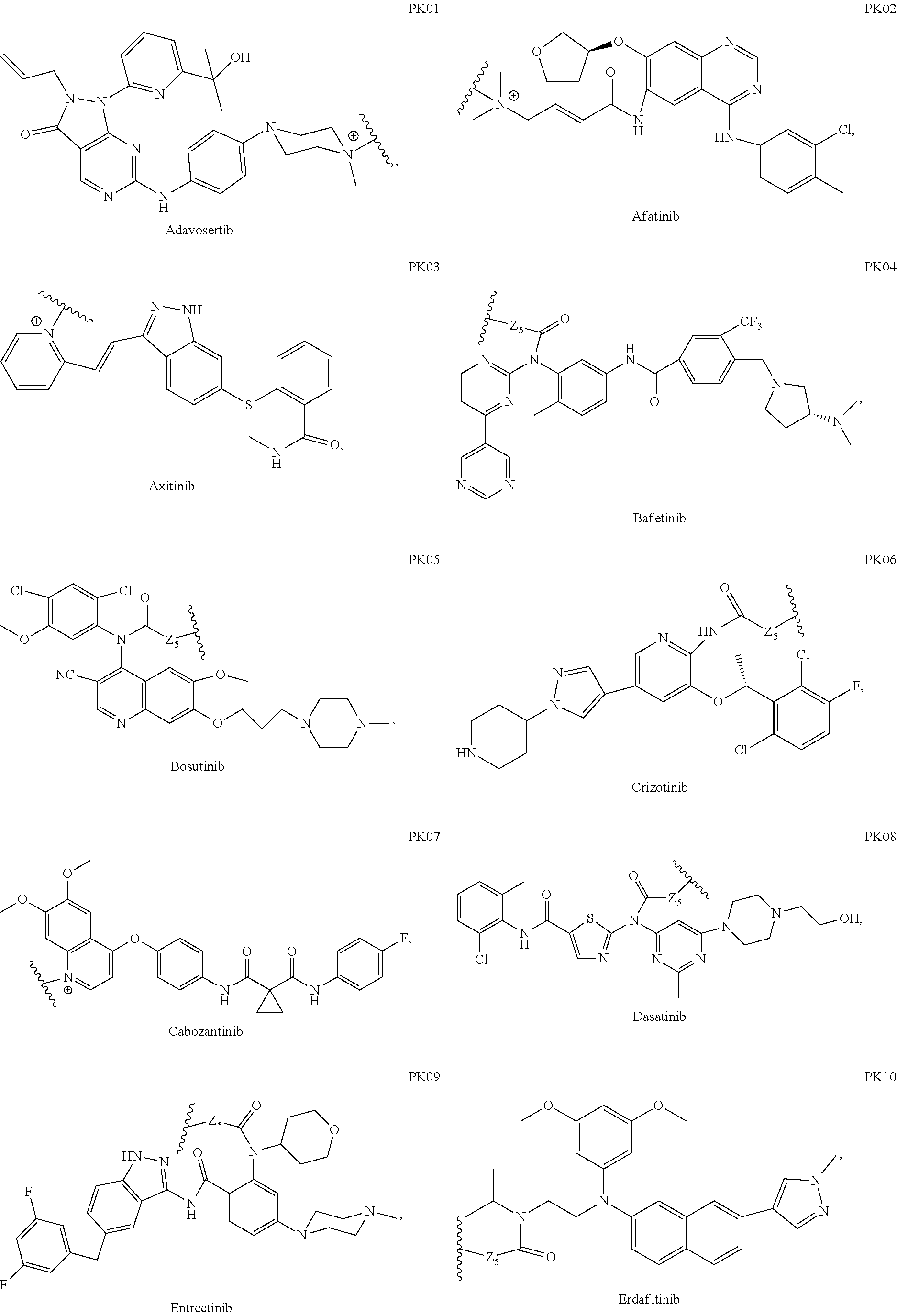

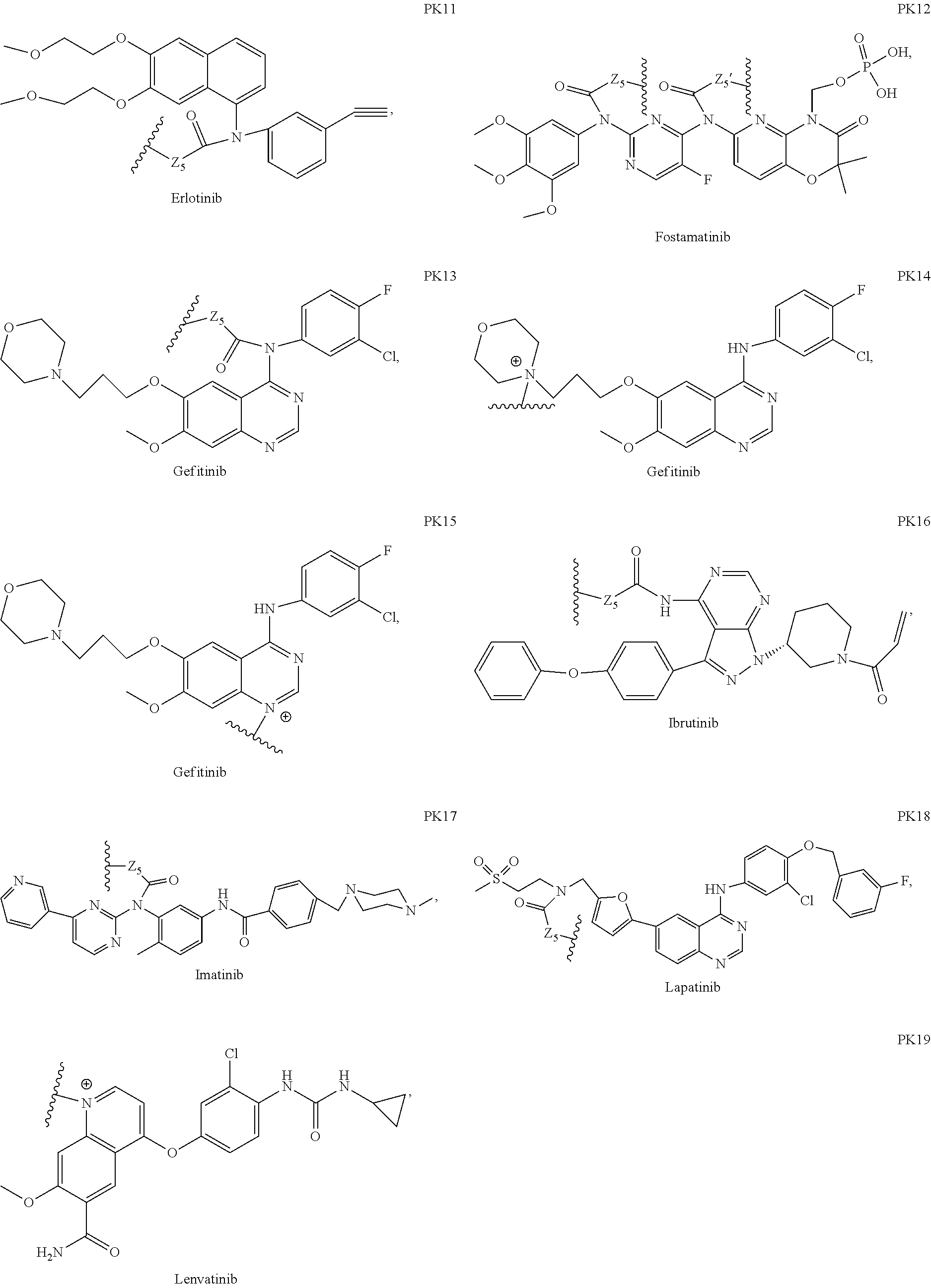

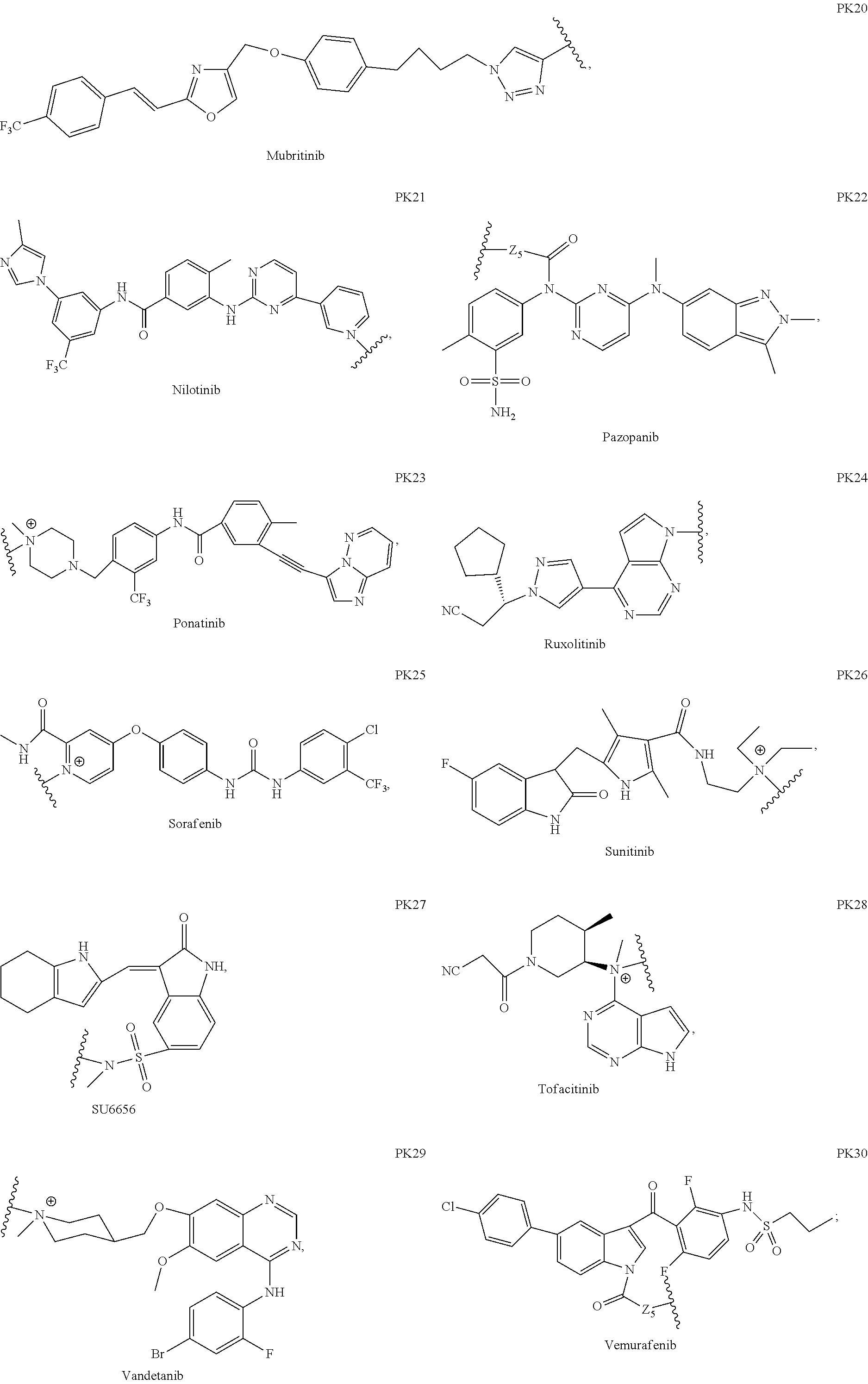

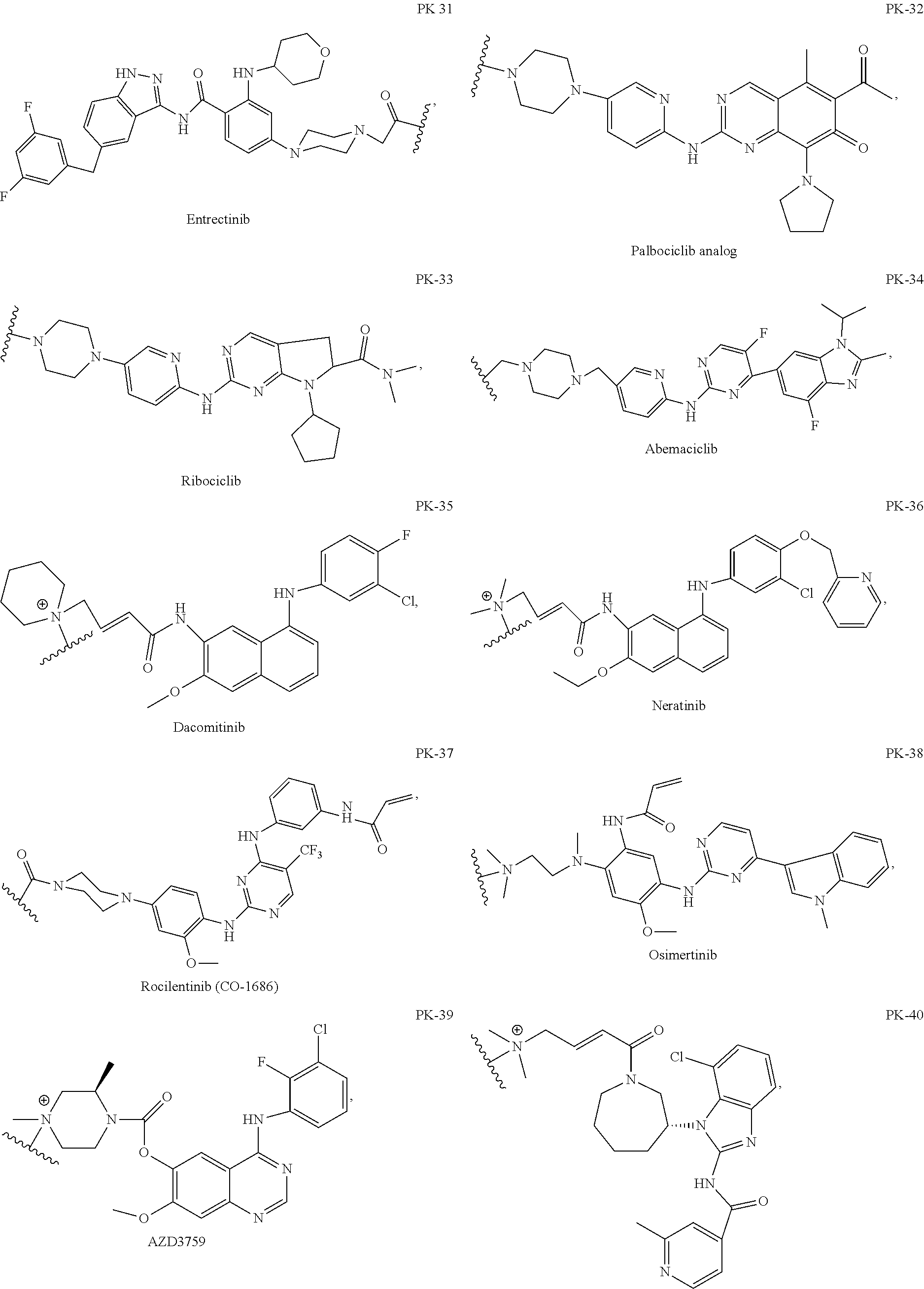

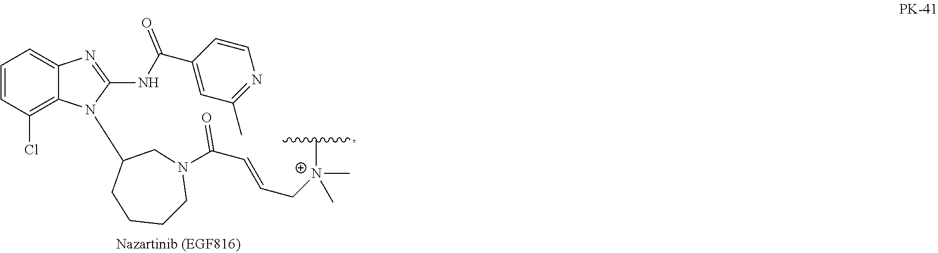

wherein Z₅ and Z₅′ are independently selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁.

In certain such embodiments, a MEK inhibitor inhibits the mitogen-activated protein kinases MEK1 and/or MEK2 which is often overactive in some cancers. MEK inhibitors are especially used for treatment of BRAF-mutated melanoma, and KRAS/BRAF mutated colorectal cancer, breast cancer, and non-small cell lung cancer (NSCLC). MEK inhibitors are selected from PD0325901, selumetinib (AZD6244), cobimetinib (XL518), refametinib, trametinib (GSK1120212), pimasertib, Binimetinib (MEK162), AZD8330, R04987655, RO5126766, WX-554, E6201, GDC-0623, PD-325901 and TAK-733. The preferred MEK inhibitors are selected from Trametinib (GSK1120212), Cobimetinib (XL518), Binimetinib (MEK162), selumetinib having the following formula:

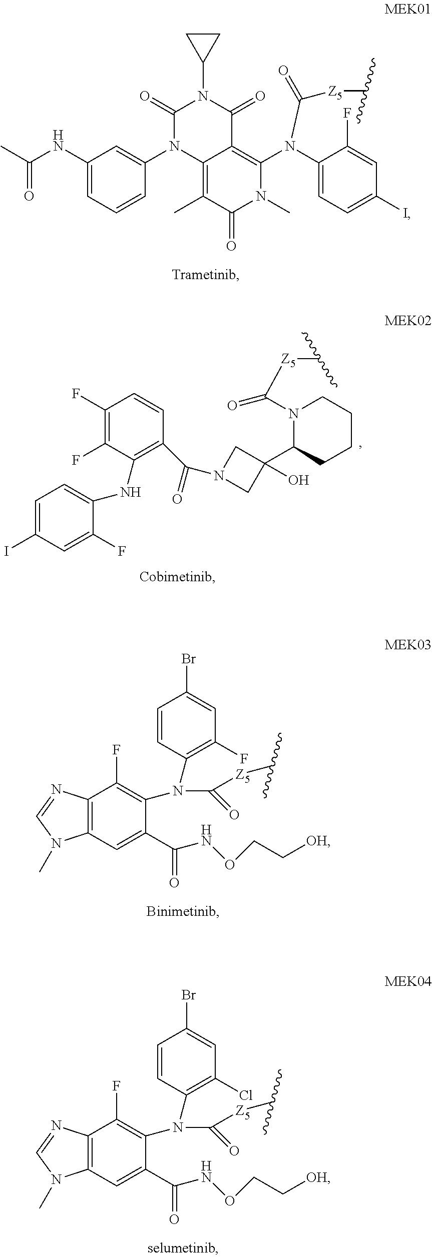

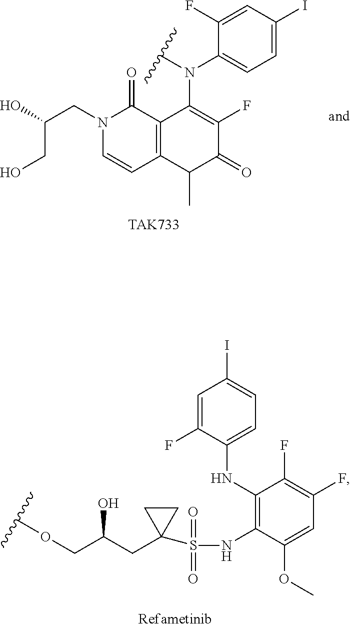

wherein Z₅ is selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁; In certain such embodiments, a proteinase inhibitor that are used as a payload is preferably selected from: Carfilzomib, Clindamycin, Retapamulin, Indibulin, as shown in the following structures:

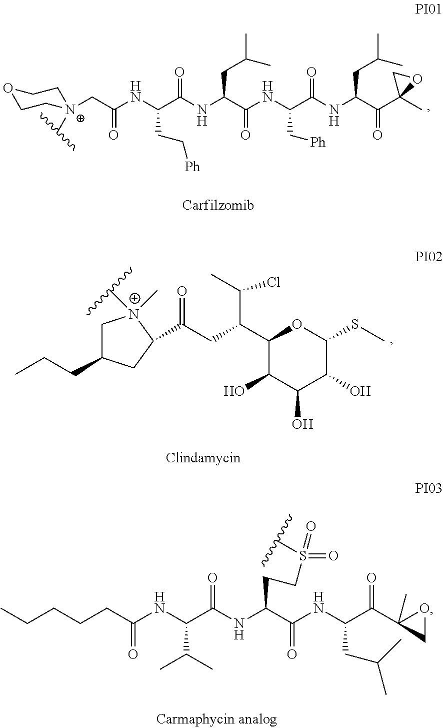

In certain such embodiments, an immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves; An immunotoxin herein is preferably conjugated via the process of the application through an amino acid having free amino, thiol or carboxyl acid group; and more preferably through N-terminal amino acid.

In addition, a certain cell receptor agonist, a cell stimulating molecule or intracellular signaling molecule can be as a chemotherapeutic/function compound conjugated to the antibody of the invention.

A cell-binding ligand or receptor agonist selected from: Folate derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinizing hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAIa-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRR₈), selected from the group consisting of Toll-like receptors' (TLR₈) ligands, C-type lectins and Nodlike Receptors' (NLR₈) ligands; Calcitonin receptor agonists; integrin receptors' and their receptor subtypes' (selected from the group consisting of αvβ₃, αᵥβ₃, αᵥβ₅, αᵥβ₆, α₆β₄, α₇β₁ α_{llb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Anticalin (a derivative of Lipocalins); Adnectins (10th FN3 (Fibronectin)); Designed Ankyrin Repeat Proteins (DARPins); Avimers; EGF receptors, or VEGF receptors' agonists; A cell-binding molecule/ligand or a cell receptor agonist selected from the following: LBO1 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for av P3 integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an a, integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) which are shown in the following structures:

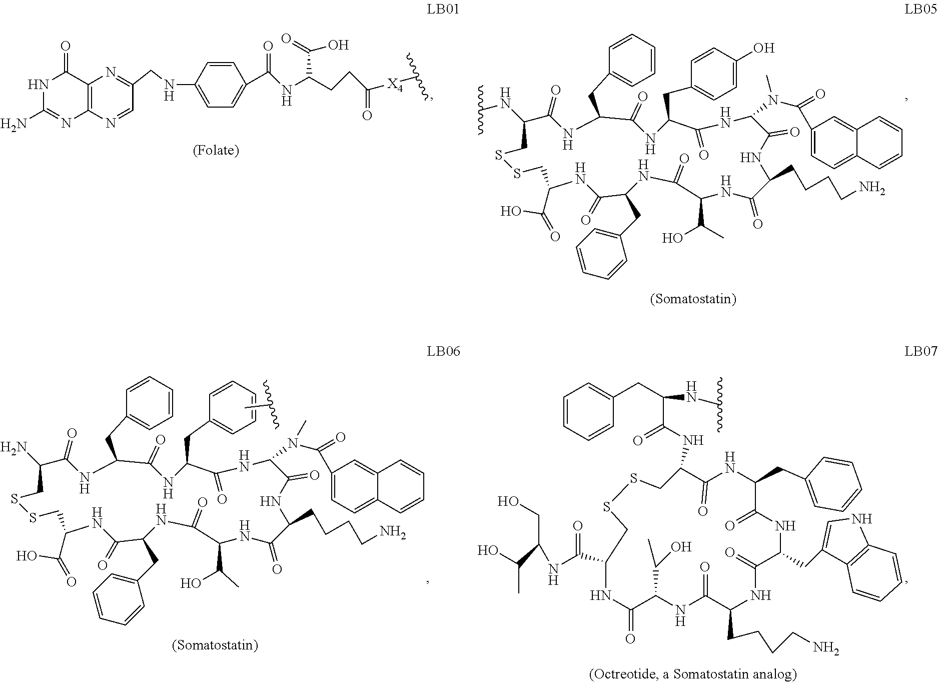

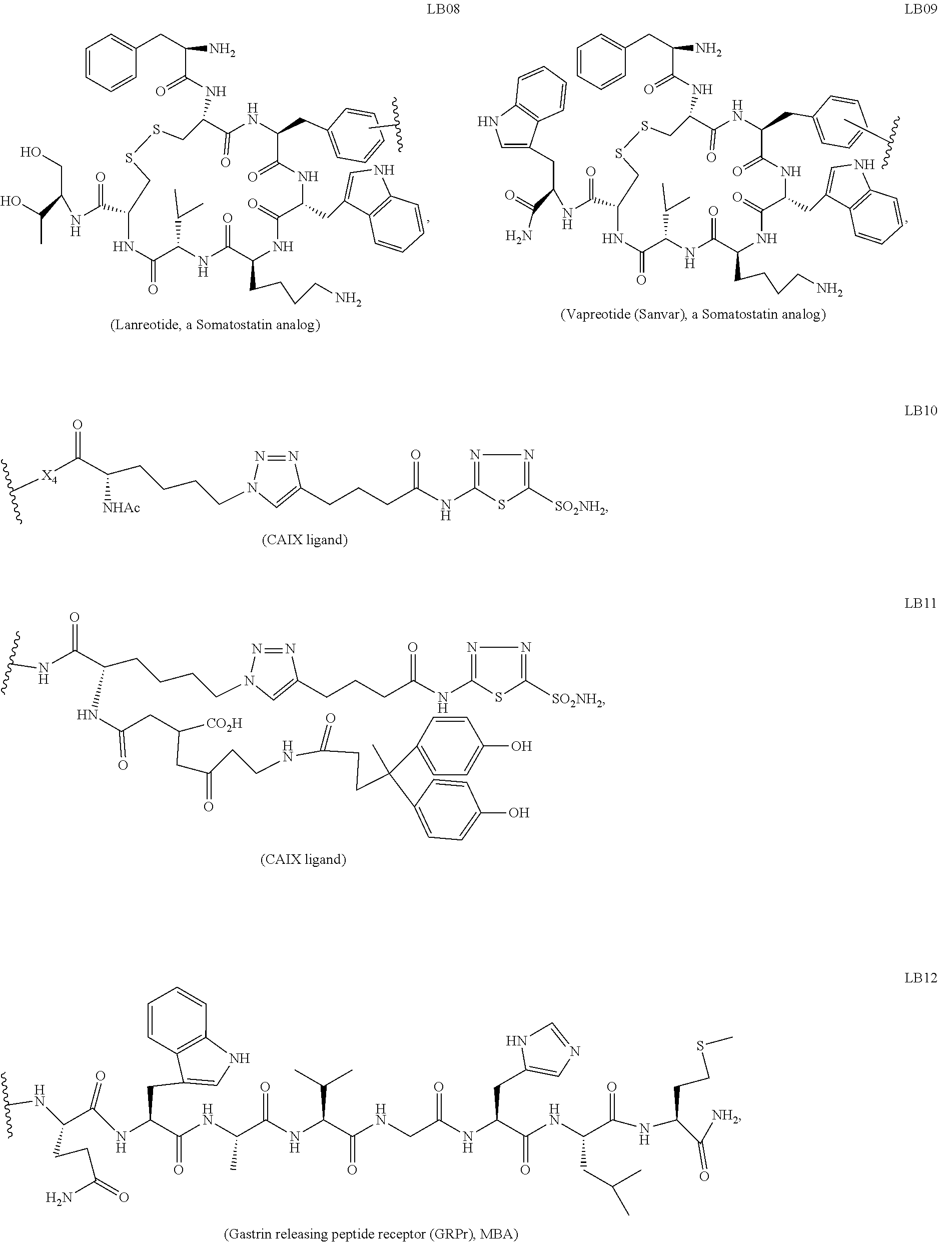

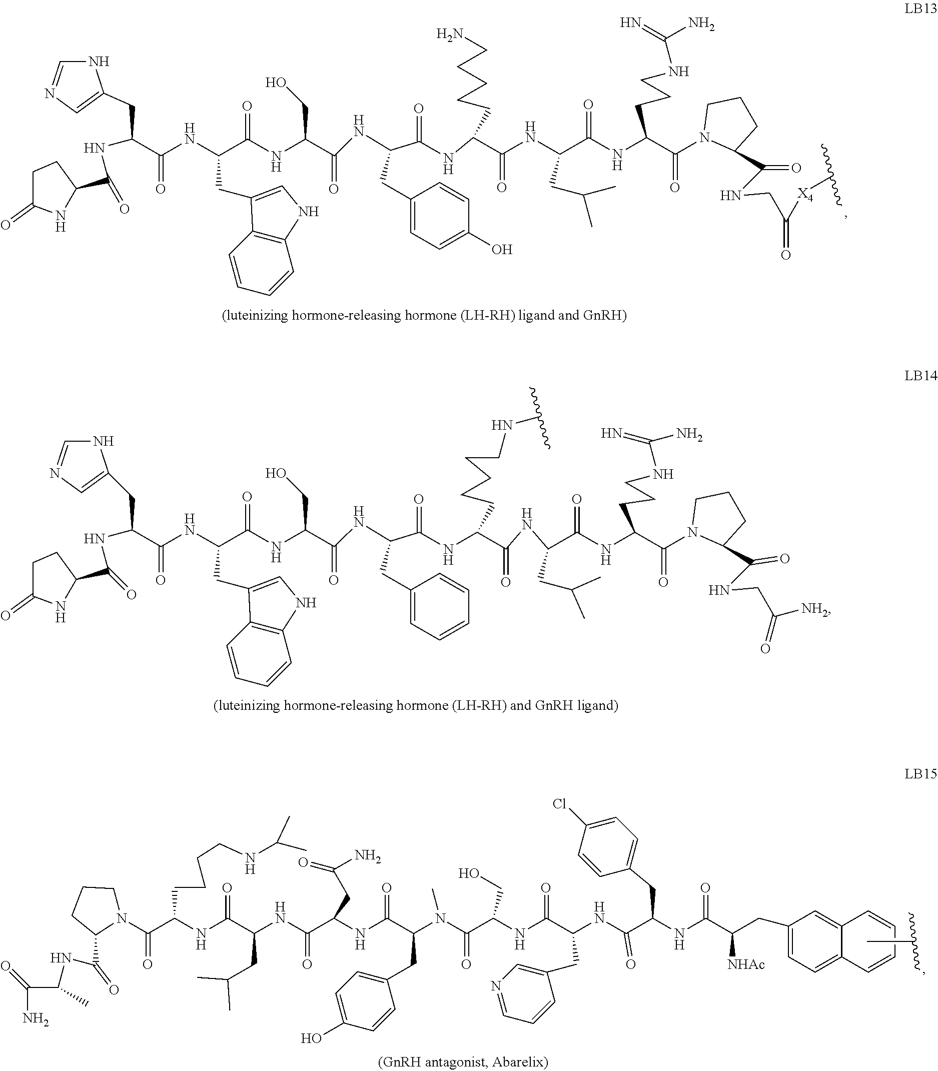

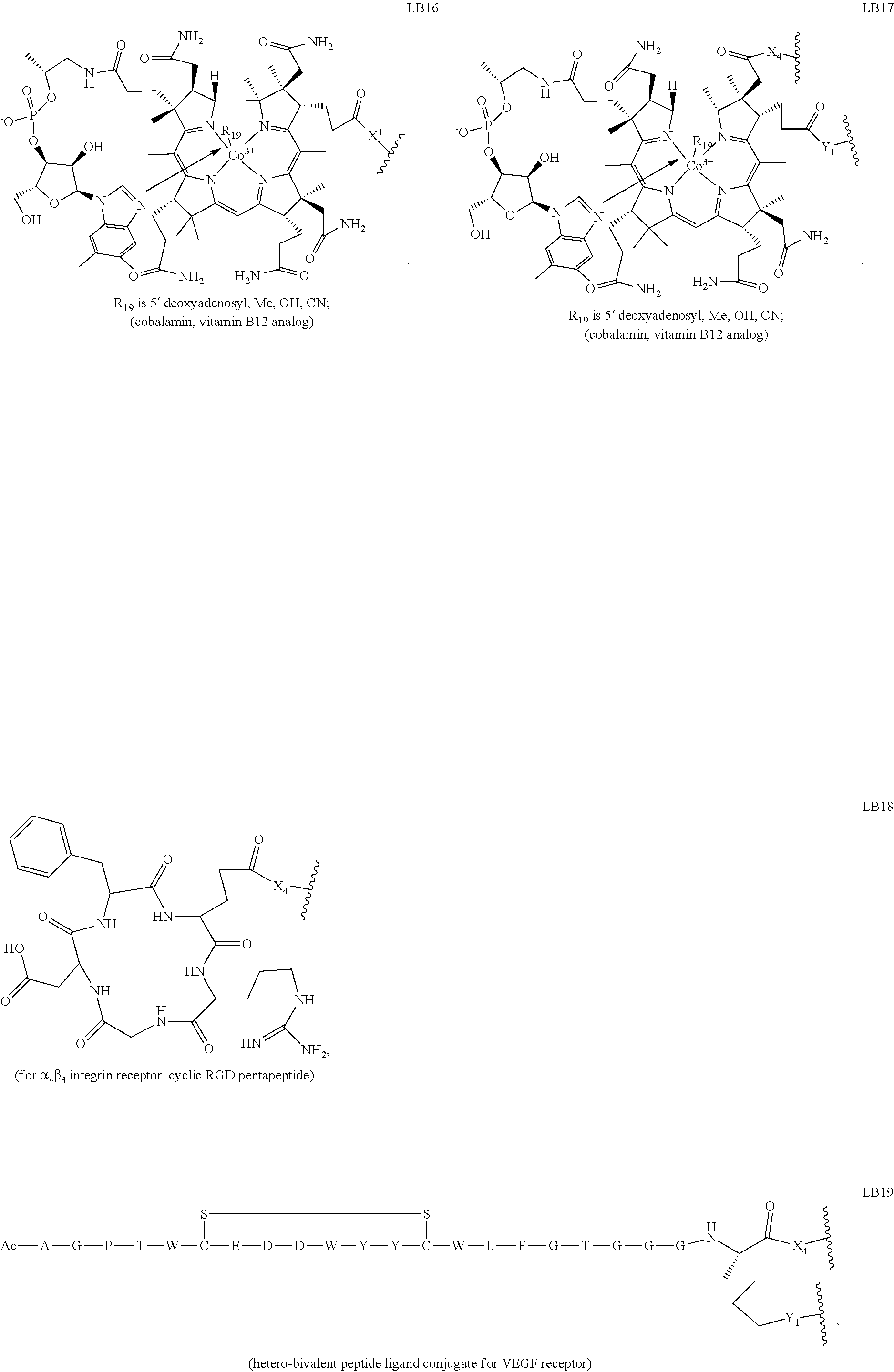

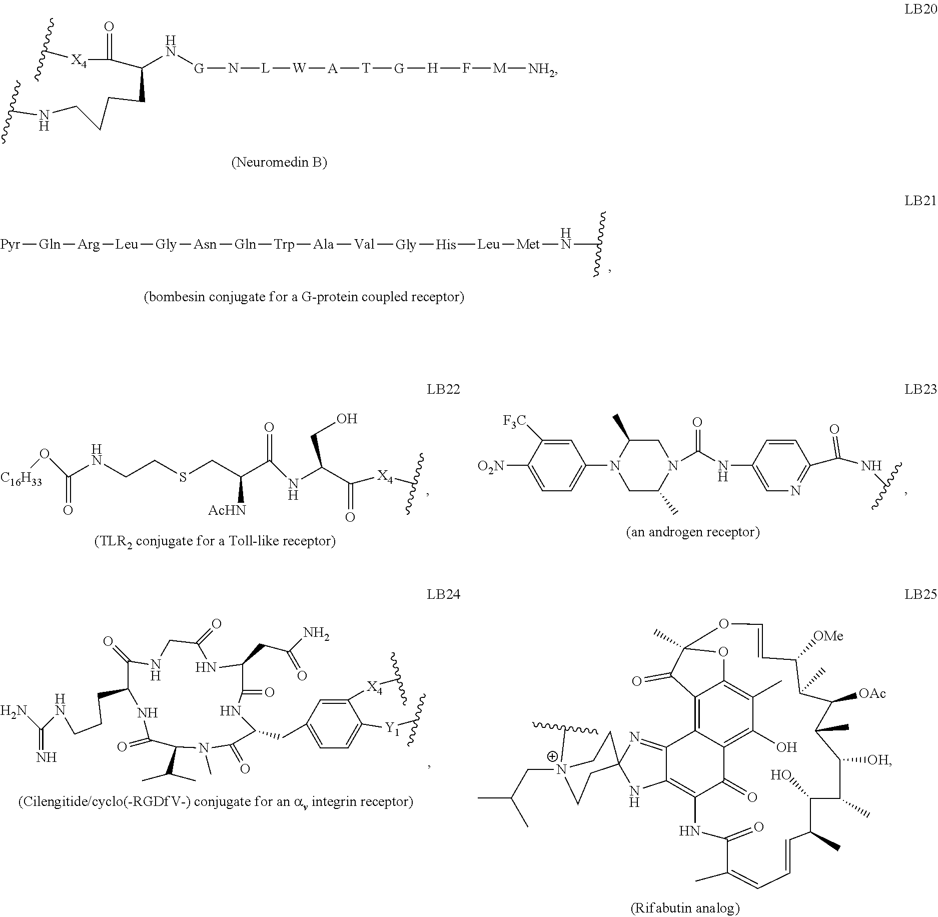

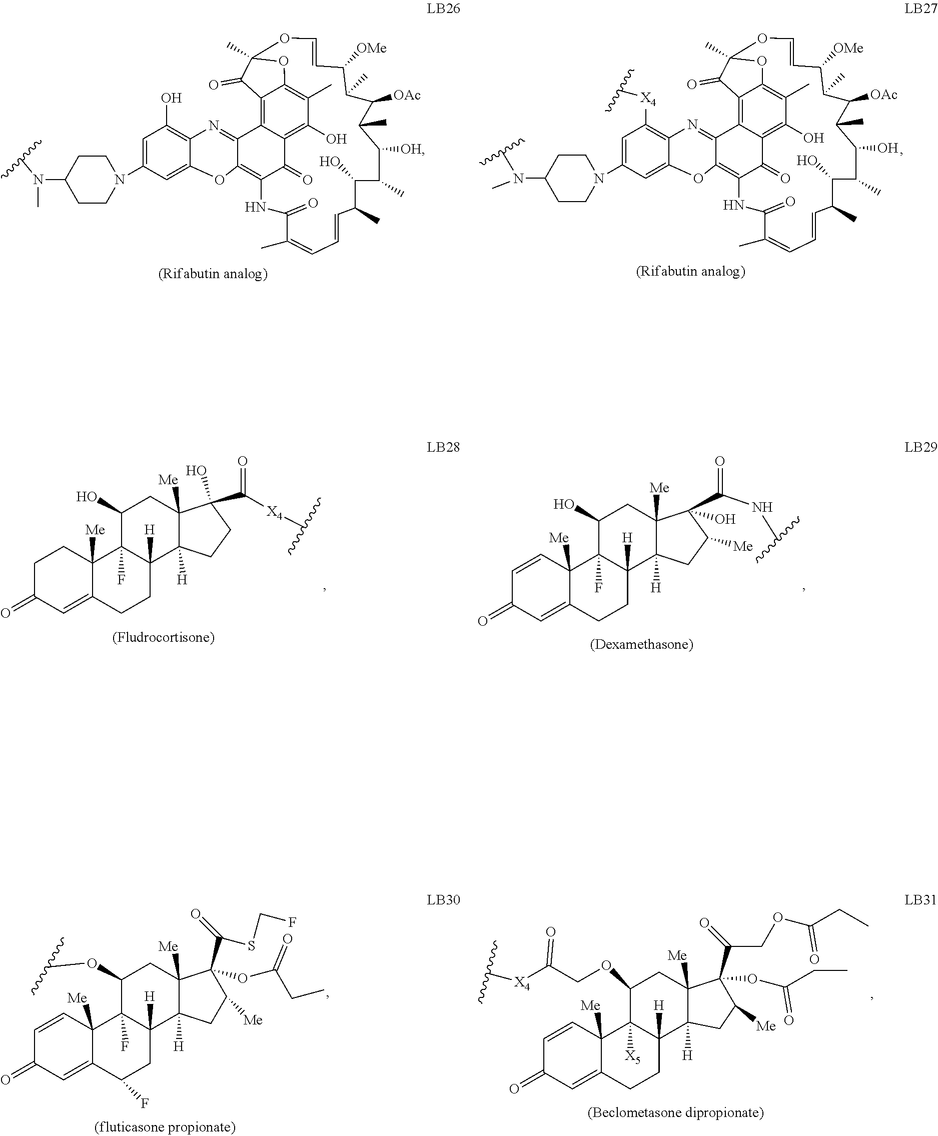

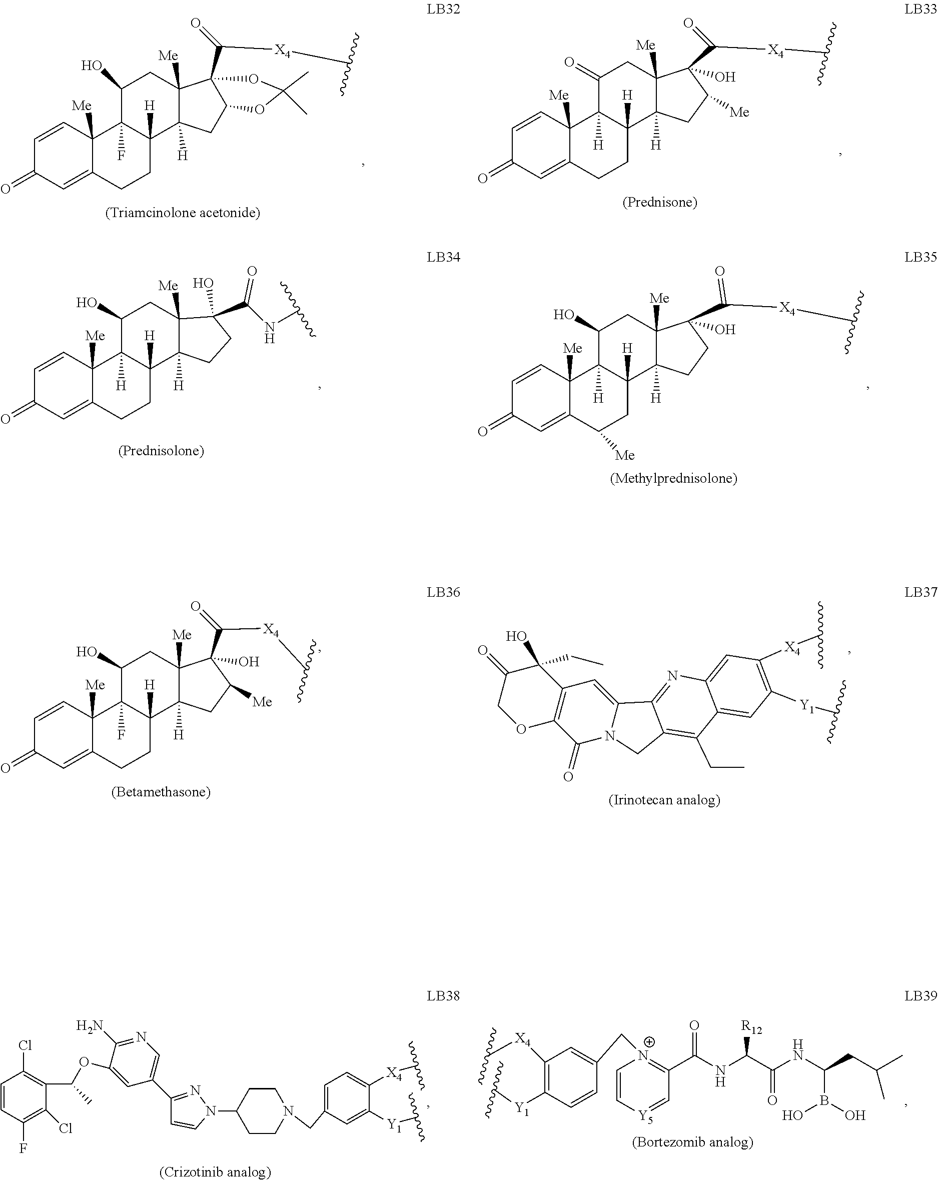

wherein Y₅, is N, CH, C(C1), C(CH₃), or C(COOR₁); R₁₂ is H, C₁-C₆ Alkyl, C₃-C₈ Ar;

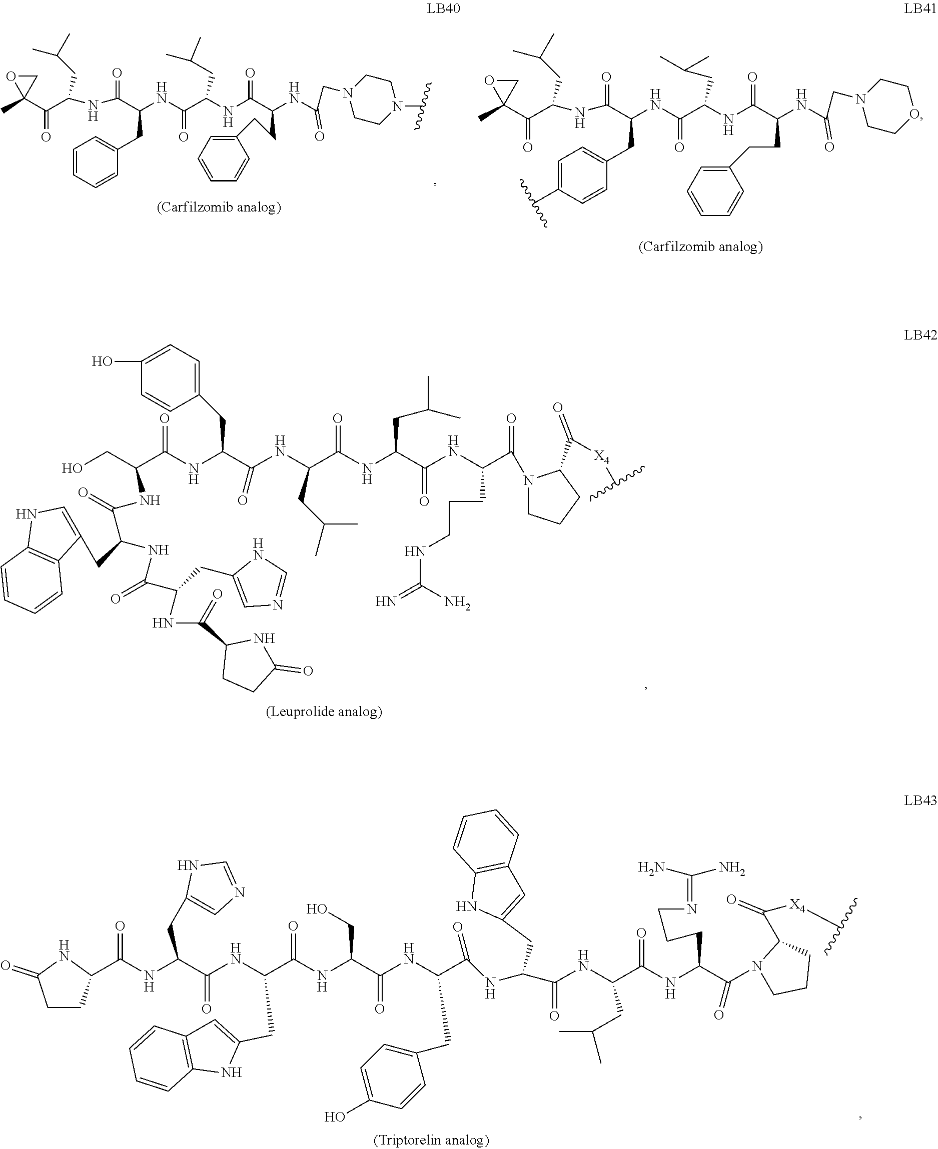

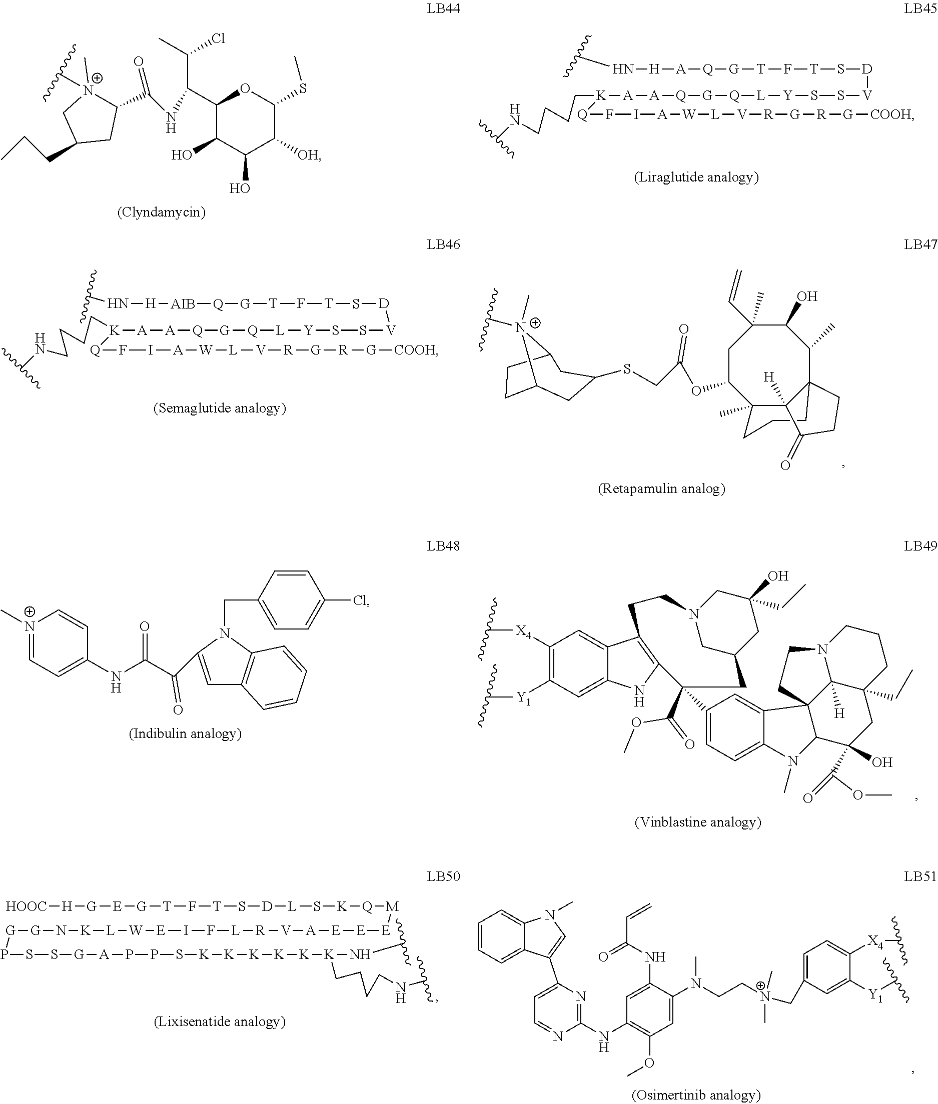

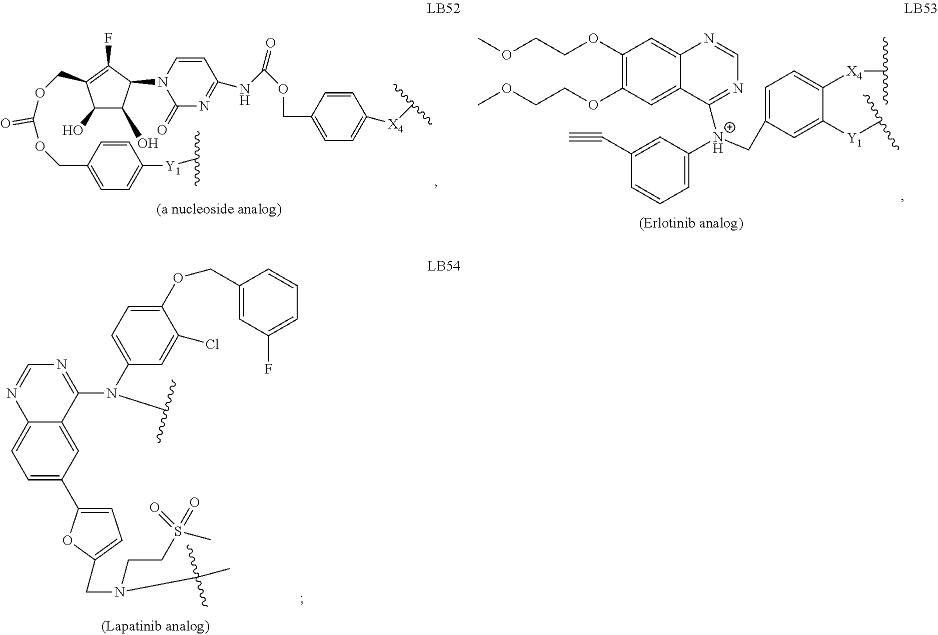

Wherein X₄,and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁, and R₁ is C₁-C₈ alkyl.

In certain embodiments, one, two or more DNA, RNA, mRNA, small interfering RNA (siRNA), microRNA (miRNA), and PIWI interacting RNAs (piRNA) can be as a chemotherapeutic/function compound conjugated to the antibody of the invention:

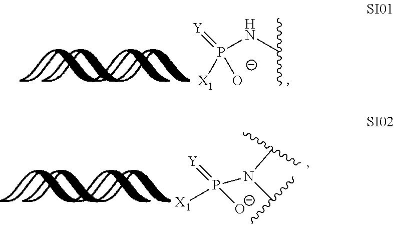

wherein “ [symbol] ” is the site to link the side chain linker of the present patent; [symbol] is single or double strands of DNA, RNA, mRNA, siRNA, miRNA, or piRNA; X₁, and Y are independently 0, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁.

In another embodiments, the linker L₁, L₂, La₁, La₂, Lb₁, Lb₂, Lc and Lc₂ are, the same or different, independently selected from O, NH, S, S—S, NHNH, N(R₃), N(R₃)N(R₃>), C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; C₂-Cₛ (2-8 carbon atoms) of esters, ether, or amide; 1-8 natural or unnatural amino acids described in the definition; polyethyleneoxy unit of formula (OCH₂CH₂)ₚ, (OCH₂CH(CH₃))ₚ, (OCH₂CH₂)ₚOR₃, (OCH₂CH(CH₃))ₚOR₃, NH(CH₂CH₂O)ₚR₃, NH(CH₂CH(CH₃)0)ₚR₃, N[(CH₂CH₂—O)ₚR₃][(CH₂CH₂O)ₚR₃′], (OCH₂CH₂)ₚCOOR₃, or CH₂CH₂(OCH₂CH₂)ₚCOOR₃, wherein p and p′ are independently an integer selected from 0 to about 1000, or combination thereof, wherein R₃ and R₃′ are independently H; C₁-Cₛ of alkyl; C₂-Cₛ of heteroalkyl, alkylcycloalkyl, heterocycloalkyl; C₃-C₈ of aryl, Ar-alkyl, heterocyclic, cycloalkyl, heteroalkylcycloalkyl, alkylcarbonyl, heteroaryl; L₁, L₂, La₁, La₂, Lb₁, Lb₂, Lc₁ and Lc₂ may independently contain a self-immolative or a non-self-immolative component, peptidyl units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals.

Preferably, the self-immolative linker component has one of the following structures:

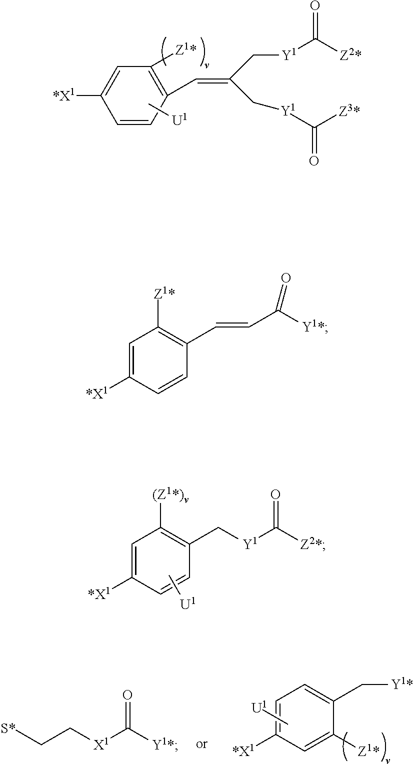

wherein the (*) atom is the point of attachment of additional spacer or releasable linker units, or the cytotoxic agent, and/or the antibody; X¹, Y¹, Z² and Z³ are independently NH, O, or S; Z¹ is independently H, NH, O or S; v is 0 or 1; U¹ is independently H, OH, C₁-C₆ alkyl, (OCH₂CH₂)nF, C1, Br, I, OR₅, SR₅, NR₅R⁵′, N═NR₅, N═R₅, NR₅R⁵′, NO2, SOR₅R⁵′, SO₂R⁵, SO₃R⁵, OSO₃R⁵, PR₅R⁵′, POR₅R⁵′, PO₂R₅R₅′, OPO(OR₅)(OR_{5′}), or OCH₂PO(OR₅(OR_{5′}) wherein R₅ and R₅′ are as defined above; preferably Rs and R₅′ are independently selected from H, C₁-C₈ alkyl; C₂-C₅ alkenyl, alkynyl, heteroalkyl; C₃~-C₈ aryl, heterocyclic, carbocyclic, cycloalkyl, heterocycloalkyl, heteroaralkyl, alkylcarbonyl; or pharmaceutical cation salts.

The non-self-immolative linker component is one of the following structures:

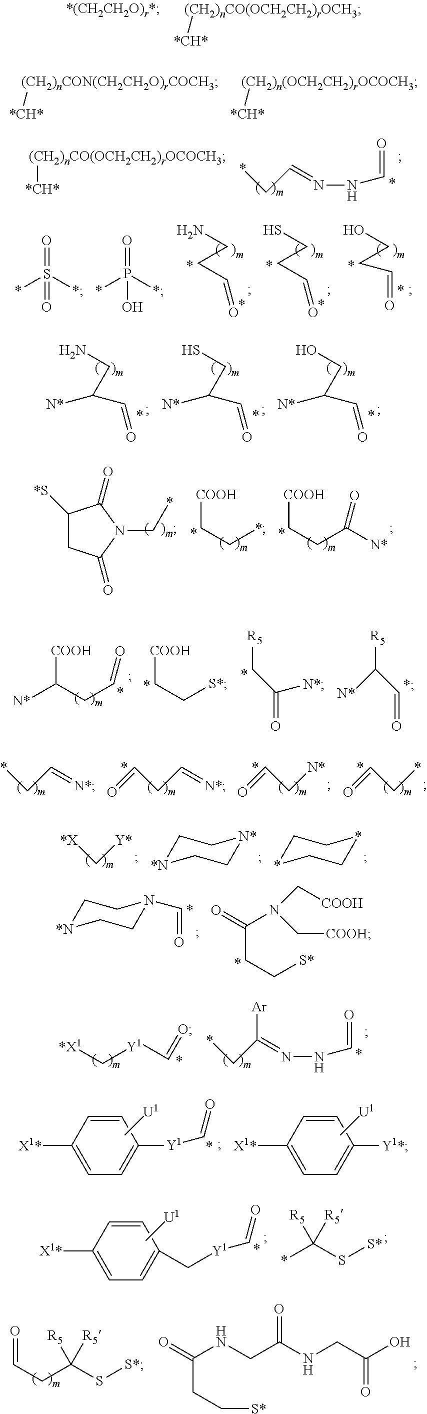

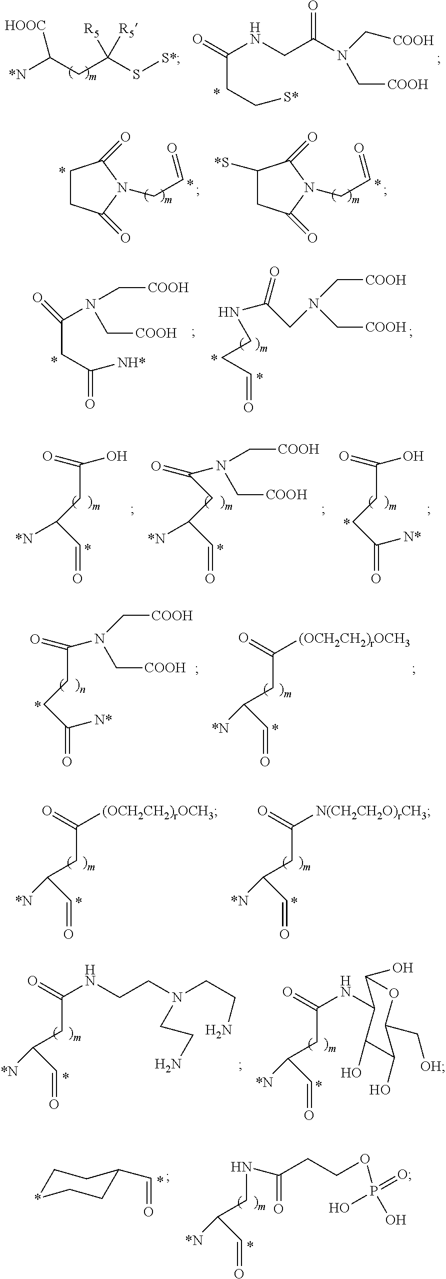

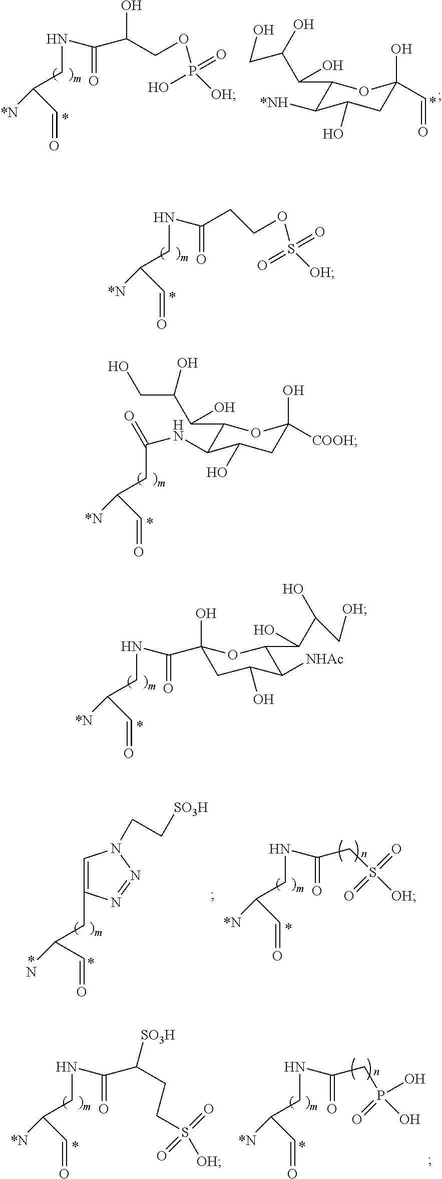

Wherein the (*) atom is the point of attachment of additional spacer R₁ or releasable linkers, the cytotoxic agents, and/or the binding molecules; X¹, Y¹, U¹, R₁, R₅, R₅′ are defined as above; r is 0~100; m and n are 0-6 independently.

More preferably, L₁, L₂, La₁, La₂, Lb₁, Lb₂, Lc and Lc₂ may independently be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites, wherein lysine, glutamic acid, aspartic acid, cysteine, or tyrosine may contain a side chain polyethyleneoxy unit of formula (OCH₂CH₂)ₚOR₃, (OCH₂CH(CH₃))ₚOR₃, NH(CH₂CH₂O)ₚR₃, C(O)(CH₂CH₂O)ₚR₃, C(O)(CH₂CH₂OCH₂CH)ₚR₃, NH(CH₂CH(CH₃)O)ₚR₃, C(O)(CH₂OCH₂CH)ₚR₃, N[(CH₂CH₂O)ₚR₃][(CH₂CH₂O)ₚ,R₃′], (OCH₂CH₂)ₚCOOR₃, or CH₂CH₂(OCH₂CH₂)ₚCOOR₃, NH(CH₂CH₂O)ₚCH₂CH₂R₃, wherein p and p′ are independently an integer selected from 0 to about 100, or combination thereof; wherein R₃ and R_{3′} are independently H, OH, NH₂, N(CH₂)₂, N(C₂H₅)₂, N(C₃H₇), C(═O)H, C(—O)CH₃, C₁-C₈ of alkyl, NH(C₄-C₇ glycoside) or NH(C₄-C₇ glycoside)₂; or combination above thereof;

Further preferably, L₁, L₂, La₁, La₂, Lb₁, Lb₂, Lc₁ and Lc₂ may independently be a releasable linker. The term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with an intracellular thiol, such as a millimolar range of abundant of glutathione inside the malignant cells.

Examples of the releasable linkers (L, L₁ or L₂) include, but not limited:
(CR₅R⁶)ₘ(Aa)ᵣ(CR₇R₈)ₙ(OCH₂CH₂)ₜ—, —(CR₅R⁶)ₘ(CR₇R₈)ₙ(Aa),(OCH₂CH₂)ₜ—, —(Aa)ᵣ(CR₅R⁶)ₘ(C R₇R₈)~(OCH₂CH₂)ₜ—, —(CR₅R⁶)ₘ(CR₇R₈)ₙ(OCH₂CH₂)ᵣ(Aa)ₜ—, —(CR₅R⁶)ₘ(CR₇=CR₈) —(CR₉R₁₀)ₙ(Aa)ₜ—(OCH₂CH₂)ᵣ—, —(CR₅R⁶)ₘ(NR₁₁CO)(Aa)ₜ(CR₉R₁₀)ₙ—(OCH₂CH₂)ₘ, —(CR₅R⁶)ₘ(Aa)ₜ—(NR₁₁CO)(CR₉R₁₀)ₙ—(OCH₂CH₂)ᵣ—, —(CR₅R⁶)ₘ(OCO)(Aa)ₜ(CR₉R₁₀)ₙ—(OCH₂CH₂)ᵣ—, —(CR₅R⁶)ₘ(OCNR₇)(Aa)ₜ(CR₉R₁₀)ₙ—(OCH₂CH₂)ᵣ—, —(CR₅R⁶)ₘ(CO)(Aa)ₜ—(CR₉R₁₀)ₙ(OCH₂CH₂)ᵣ—, —(CR₅R⁶)ₘ(NR₁₁CO)(Aa)ₜ(CR₉R₁₀)ₙ—(OCH₂CH₂)ᵣ—, —(CR₅R⁶)ₘ—(OCO)(Aa)ₜ(CR₉R₁₀)ₙ—(OCH₂CH₂)ᵣ—, —(CR₅R⁶)ₘ(OCNR₇)(Aa)ₜ(CR₉R₁₀)ₙ—(OCH₂CH₂)ᵣ—, —(CR₅R⁶)ₘ(CO)(Aa)ₜ(CR₉R₁₀)ₙ.(OCH₂CH₂)ᵣ—, —(CR₅R⁶)ₘ-phenyl-CO(Aa)ₜ(CR₇R₈)ₙ—, —(CR₅R⁶)ₘ-furyl-CO(Aa)ₜ(CR₇R₈)ₙ—, —(CR₅R⁶)ₘ-oxazolyl-CO(Aa)ₜ(CR₇R₈)ₘ—, —(CR₅R⁶)ₘthiazolyl-CO—(Aa)ₜ(CCR₇R₈)ₙ—, —(CR₅R⁶)ₜ-thienyl-CO—(CR₇R₈)ₘ—, —(CR₅R⁶)ₜ-imidazolyl-CO—(CR₇R₈)ₘ—, —(CR₅R⁶)ₜ-morpholino-CO(Aa)ₜ—(CR₇R₈)ₘ—, —(CR₅R⁶)tpiperazino-CO(Aa)ₜ(CR₇R₈)ₙ—, —(CR₅R⁶)ₜ—N-methyl-piperazin-CO(Aa)ₜ.(CR₇R₈)ₘ—, —(CRsR)ₘ-(Aa)ₜphenyl-, —(CR₅R⁶)ₘ—(Aa)ₜfuryl-, —(CR₅R⁶)ₘ-oxazolyl(Aa)ₜ—, —(CR₅R⁶)ₘ-thiazolyl(Aa)ₜ—, —(CR₅R⁶)ₘ-thienyl-(Aa)ₜ—, —(CR₅R⁶)ₘ-imidazolyl(Aa)ₜ—, —(C R₅R⁶)ₘ-morpholino-(Aa)ₜ—, —(CR₅R⁶)ₘ—piperazino-(Aa)ₜ—, —(CR₅R⁶)ₘN-methylpiperazino-(Aa)ₜ—,
—K(CR₅R⁶)ₘ(Aa)ᵣ(CR₇R₈)ₙ(OCH₂CH₂)ₜ—, —K(CR₅R⁶)ₘ(CR₇R₈)ₙ—(Aa)ᵣ(OCH₂CH₂)ₜ—, —K(Aa)ᵣ(CR₅R⁶)ₘ—(CR₇R₈)~(OCH₂CH₂)ₜ—, —K(CR₅R⁶)ₘ(CR₇R₈)ₙ—(OCH₂CH₂)ᵣ(Aa)ₜ—, —K(CR₅R⁶)ₘ(CR₇=CR₈)(CR₉R₁₀)ₙ—(Aa)ₜ(OCH₂CH₂)ᵣ—, —K(CR₅R⁶)ₘ—(NR₁₁CO)(Aa)ₜ(CR₉R₁₀)ₙ(OCH₂CH₂)ₘ, —K(CR₅R⁶)ₘ(Aa)ₜ(NR₁₁CO)—(CR₉R₁₀)ₙ(OCH₂—CH₂)ᵣ—, —K(CR₅R⁶)ₘ(OCO)(Aa)ₜ(CR₉R₁₀)ₙ—(OCH₂CH₂)ᵣ—, —K(CR₅R⁶)ₘ(OCNR₇)(Aa)ₜ—(CR₉R₁₀)ₙ—(OCH₂CH₂)ᵣ—, —K(CR₅R⁶)ₘ(CO)(Aa)ₜ—(CR₉R₁₀)ₙ(OCH₂CH₂)ᵣ—, —K(CR₅R⁶)ₘ(NR₁₀CO)—(Aa)ₜ(CR₉R₁₀)~(OCH₂CH₂)ᵣ—, —K(CR₅R⁶)ₘ—(OCO)(Aa)ₜ(CR₉R₁₀)ₙ(OCH₂CH₂)ᵣ—, —K(CR₅R⁶)ₘ(OCNR₇)—(Aa)ₜ(CR₉R₁₀)ₙ(OCH₂CH₂)ₘ, —K(CR₅R⁶)ₘ(CO)(Aa)ₜ(CR₉R₁₀)ₙ—(OCH₂CH₂)ᵣ—, —K(CR₅R⁶)ₘ- phenyl-CO—(Aa)ₜ(CR₇R₈)ₘ—, —K—(CR₅R⁶)ₘ-furyl-CO(Aa)ₜ.(CR₇R₈)ₙ—, —K(CR₅R⁶)ₘ-oxazolyl-CO(Aa)ₜ(CR₇R₈)ₙ—, —K(CR₅R⁶)mthiazolyl-CO(Aa)ₜ—(CR₇R₈)ₙ—, —K(CR₅R⁶)ₜ-thienyl-CO(CR₇R₈)ₘ—, —K(CR₅R⁶)timidazolyl-CO—(CR₇R₈)ₙ—, —K(CR₅R⁶)tmorpholino-CO(Aa)ₜ(CR₇R₈)ₘ—, —K(CR₅R⁶)tpiperazino-CO(Aa)ₜ.(CR₇R₈)ₘ—, —K(CR₅R⁶)ᵣ—N-methylpiperazinCO(Aa)ₜ(CR₇R₈)ₙ—, —K(CR₅R)ₘ(Aa)tphenyl, —K—(CR₅R⁶)ₘ—(Aa)ₜfuryl-, —K(CR₅R⁶)ₘ-oxazolyl(Aa)ₜ—, —K(CR₅R⁶)ₘ-thiazolyl(Aa)ₜ—, —K(CR₅R⁶)ₘ-thienyl-(Aa)ₜ—, —K(CR₅R⁶)ₘ—imidazolyl(Aa)ₜ—, —K(CR₅R⁶)ₘ-morpholino(Aa)ₜ—, —K(CR₅R⁶)ₘpiperazino-(Aa)ₜG, —K(CR₅R⁶)ₘN-methylpiperazino-(Aa)ₜ—; wherein m, n, R₃, R₄, and R₅ are described above; Aa is an amino acid, t and r are 0-100 independently; R₆, R₇, and Rₛ are independently chosen from H; halide; C₁-C₈ of alkyl, aryl, alkenyl, alkynyl, ether, ester, amine or amide, which optionally substituted by one or more halide, CN, NR₁R₂, CF₃, ORI, Aryl, heterocycle, S(O)R¹, SO₂R₁, —CO₂H, —SO₃H, —OR₁, —CO₂R¹, —CONR₁, —PO₂R₁R₂, —PO₃H or P(O)R₁R₂R₃; K is NRI, —SS—, —C(═O)—, —C(═O)NH—, —C(═O)O—, —C═NH—O—, —C═N—NH—, —C(═O)NH—NH—, O, S, Se, B or C₃-C₆ heteroaromatic group.

Example structures of the components of the linker L₁, L₂, La₁, La₂, Lb₁, Lb₂, Lc₁ and Lc₂ may independently contain one or several of the following structures:

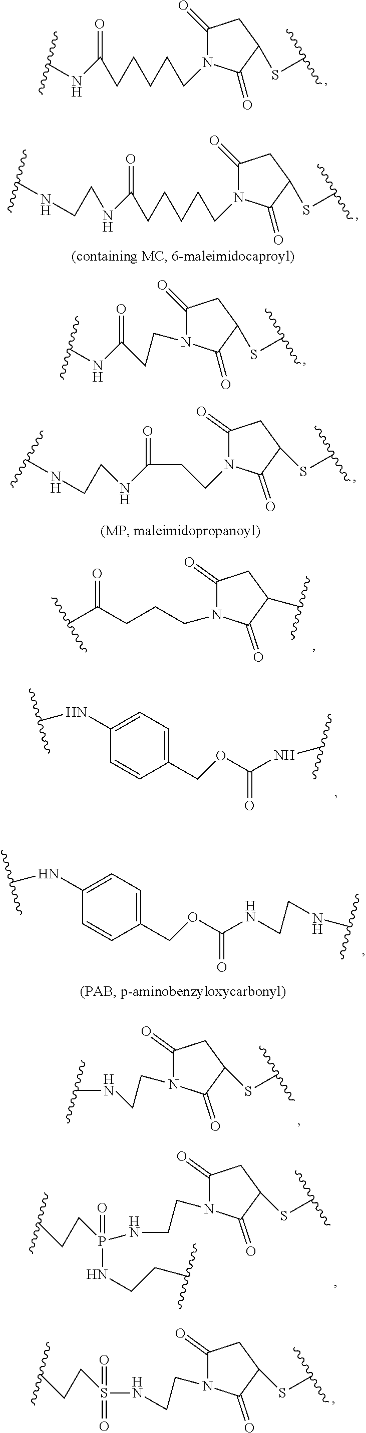

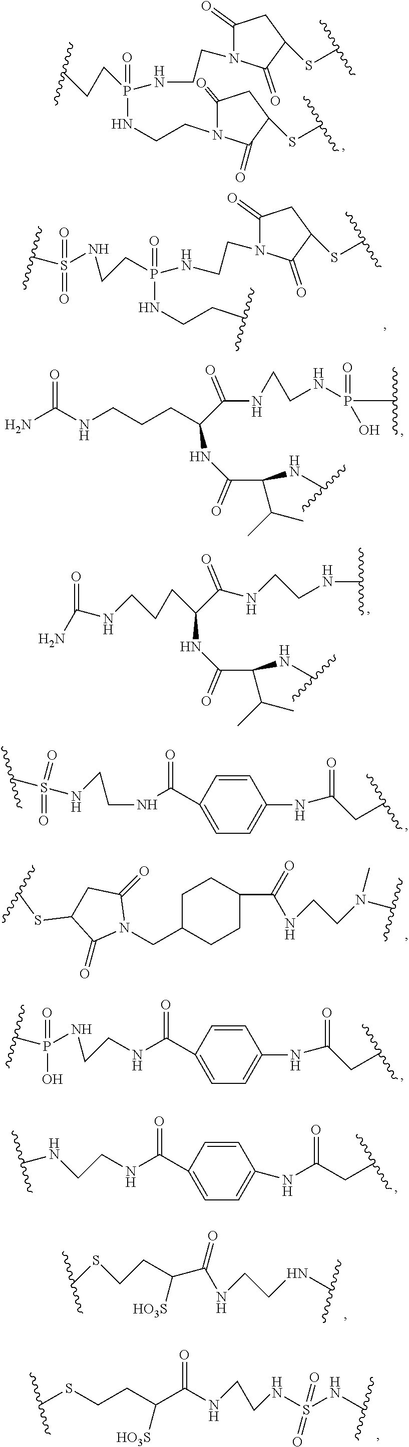

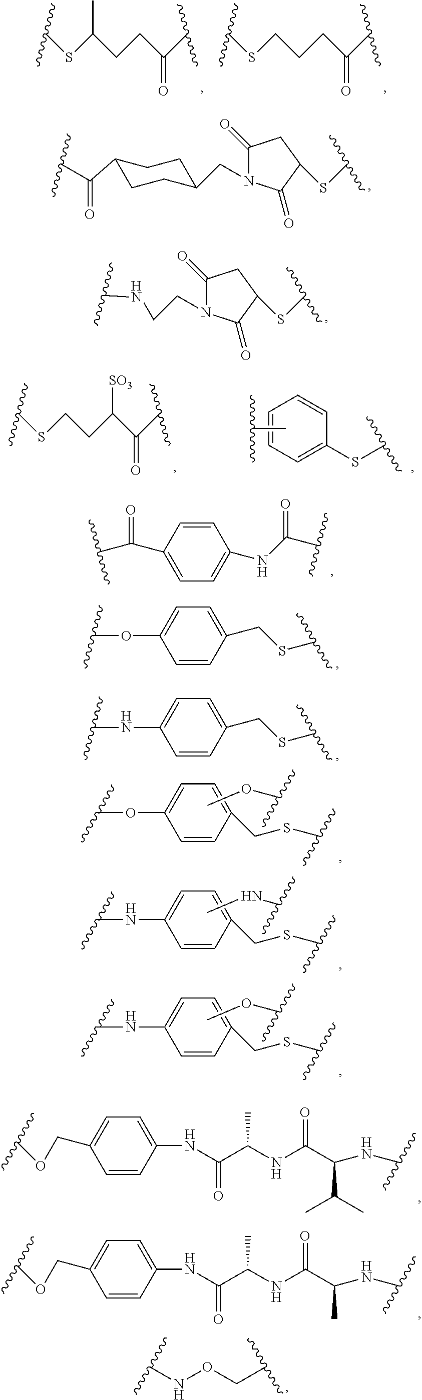

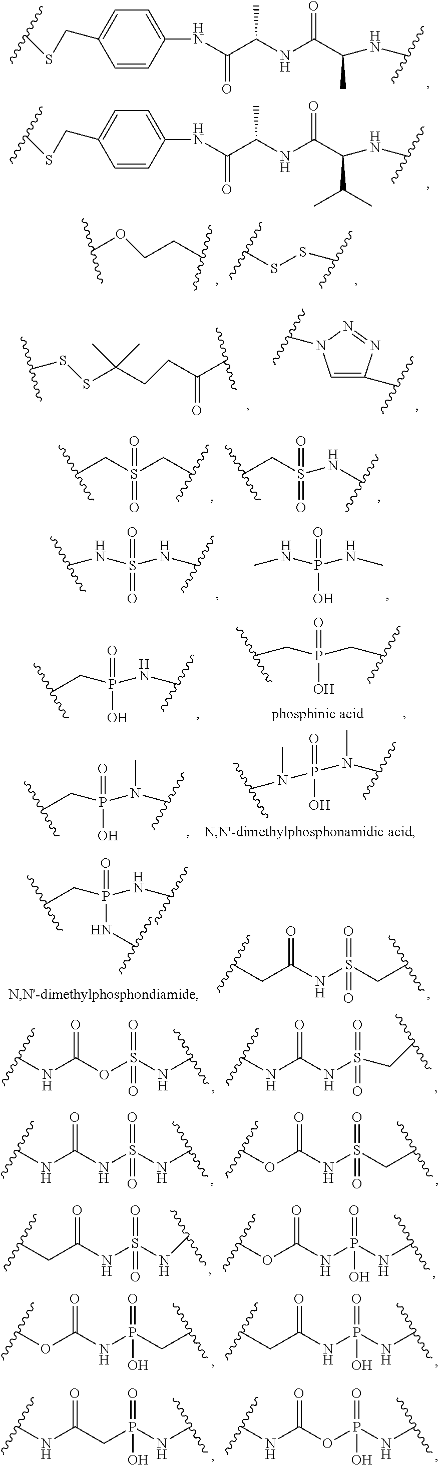

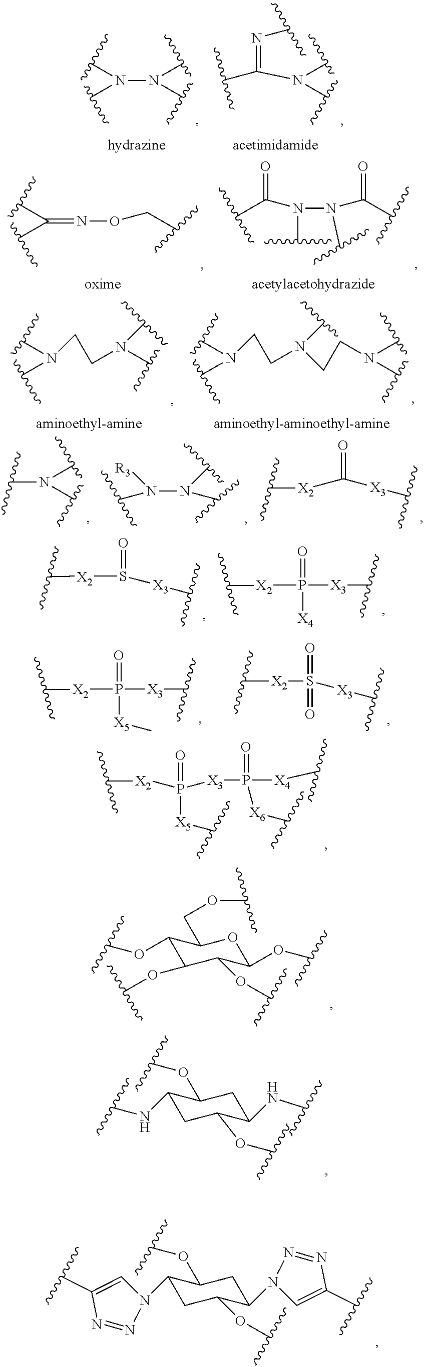

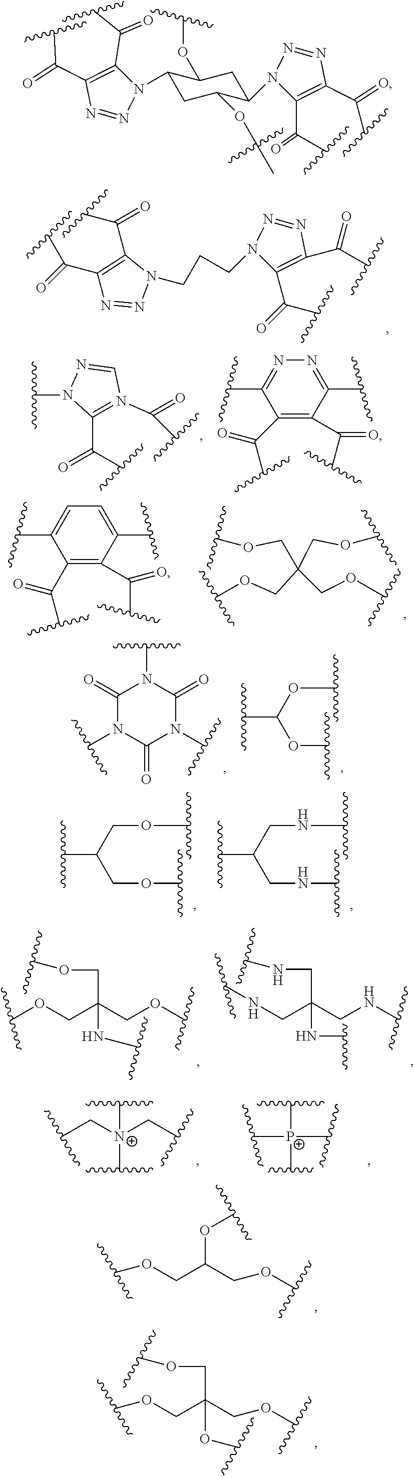

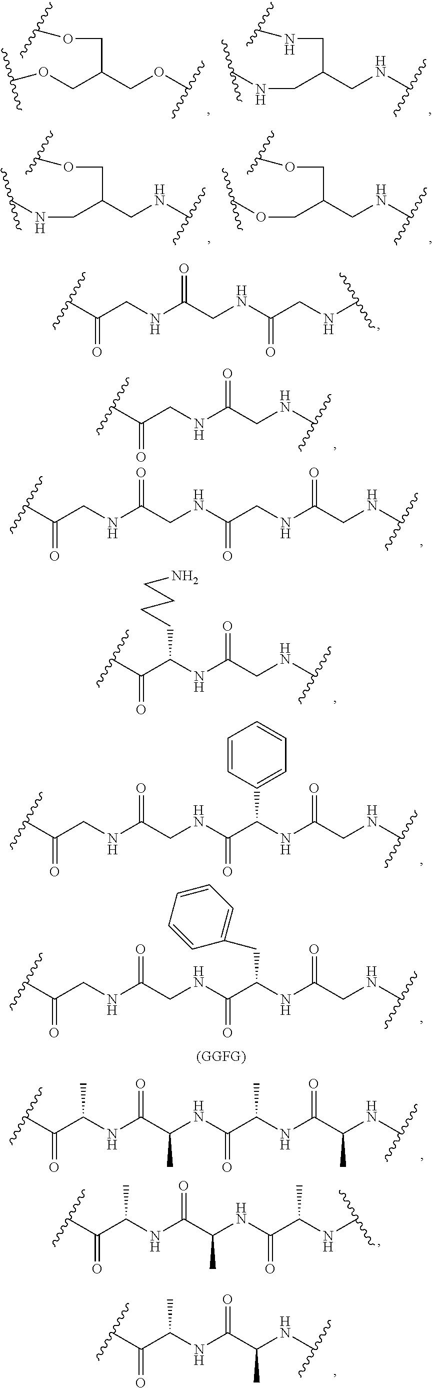

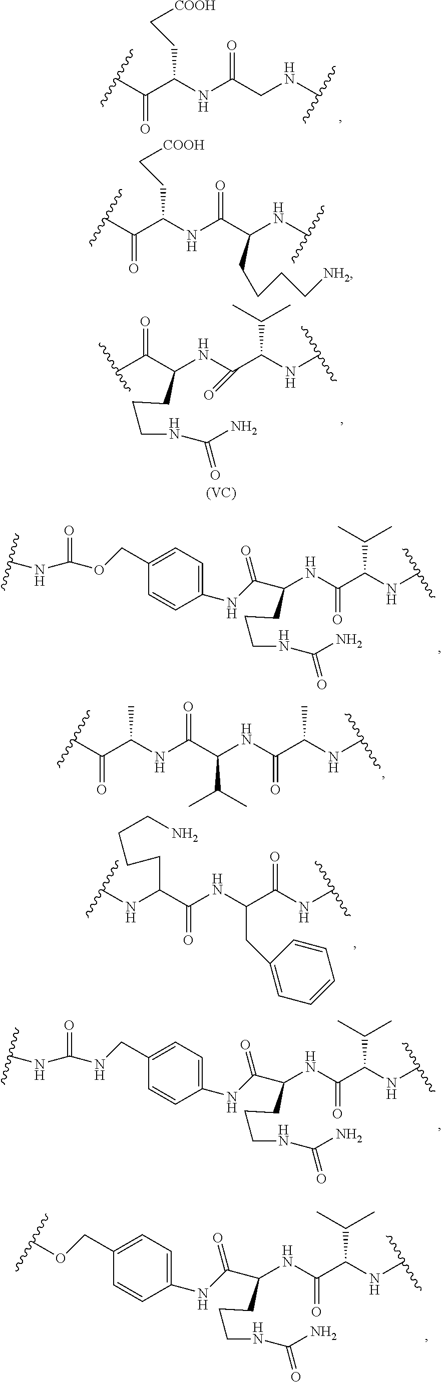

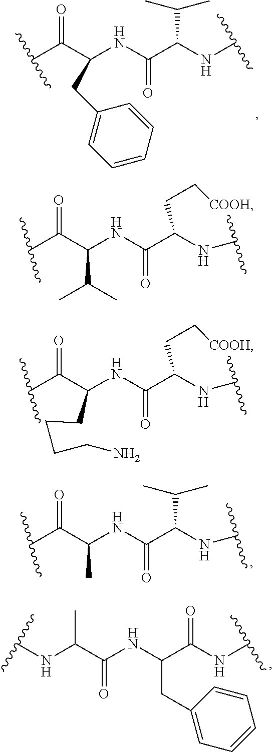

or a combination above thereof, wherein [symbol] is the site of linkage;, X₂, X₃, X₄, X₅, or X₆, are independently
selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R₁₂r are independently H;C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 100.

In another embodiments, the L₁, L₂, Lat, La₂, Lb₁, Lb₂, E₁, E₂, Lv₁′, Lv₂′, Lc₁ and Lc₂ that are jointly constructed in the structures of Formula (I), (II) and (III) are (IV) are accordingly selected from the following preferences:

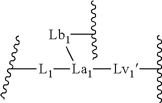

wherein the structure of
in formula (I) is preferably having the structure of formula (Ta); wherein the structure of

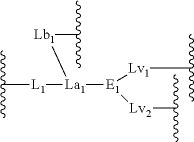

in formula (II) is preferably having the structure of formula (Ib) or (Ic); wherein the structure of

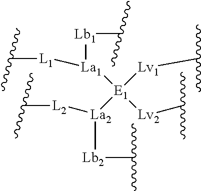

in formula (III) is preferably having the structure of formula (Id), (Ie), (If) or (Ig), or wherein

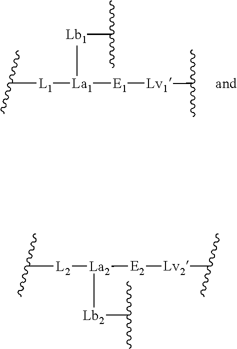

in formula (IV), is preferably having the structure of formula (Ia), illustrated as following:

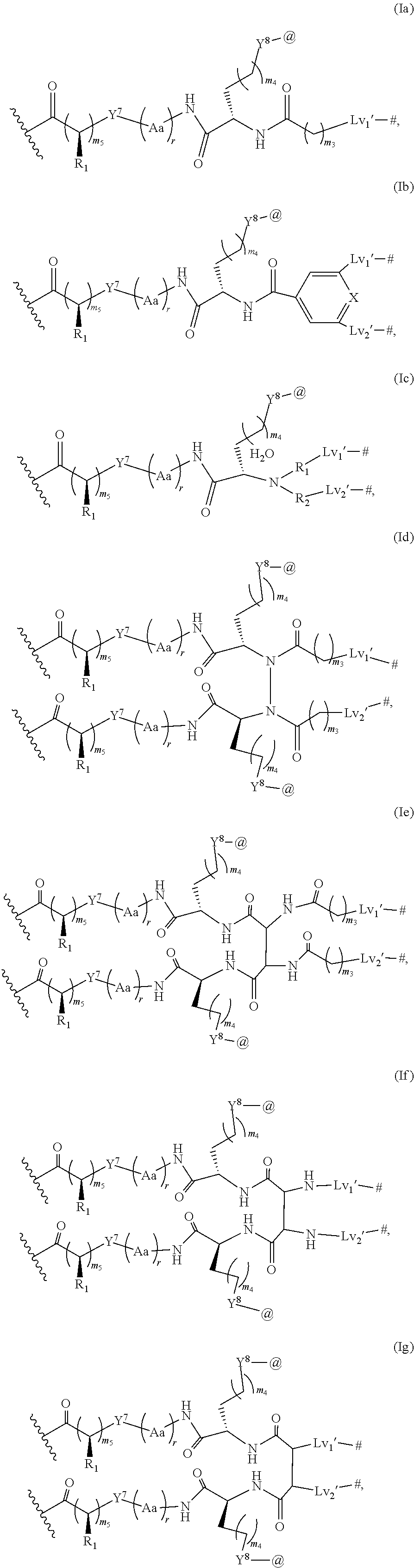

Wherein “ [symbol] ” is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; Aa is L-or D- natural or unnatural amino acids; “@” is a site that links Lc₁ or Lc₂ described in the formula (I), (II), (III) and (IV).
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0-12; when r is not 0, (Aa)ᵣ is the same or different amino acids or peptide units;
m₁ =1-18; m₂=1-100; m₃=1-8; m₄=0-8; m₅=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H4NH, CH₂;
Y₈ is NHC(═O), NHS(O₂), NH(SO), NHS(O₂)NH, NHP(O)(OH)NH, C(O)NH, OC(O)NH, NHC(O)NH, C(O), N, NH, CH₂, or CH;
Lv₁′ and Lv₂′ are independently selected from:

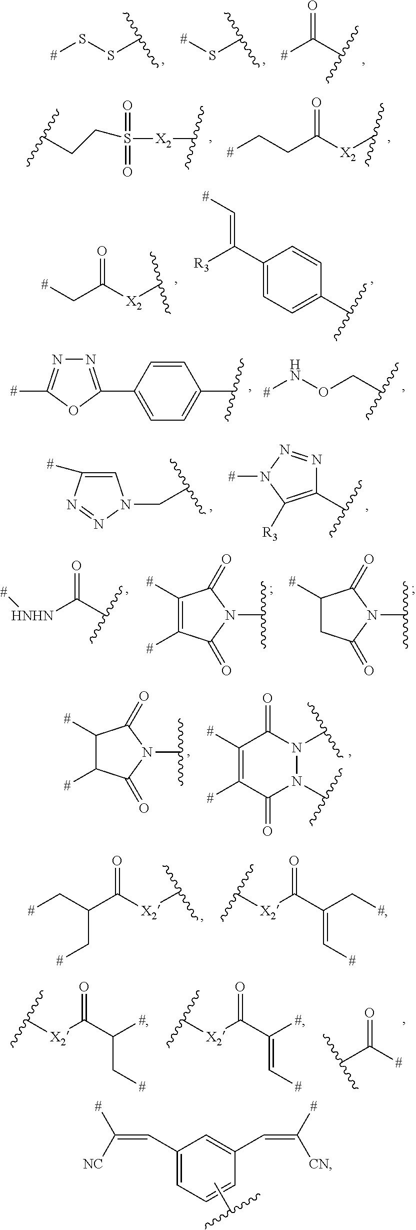

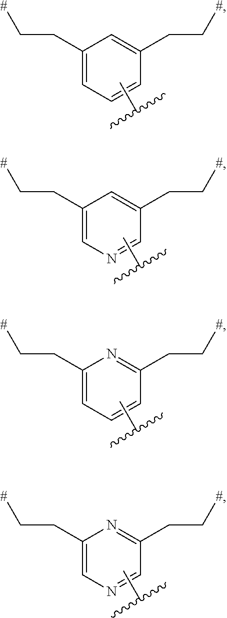

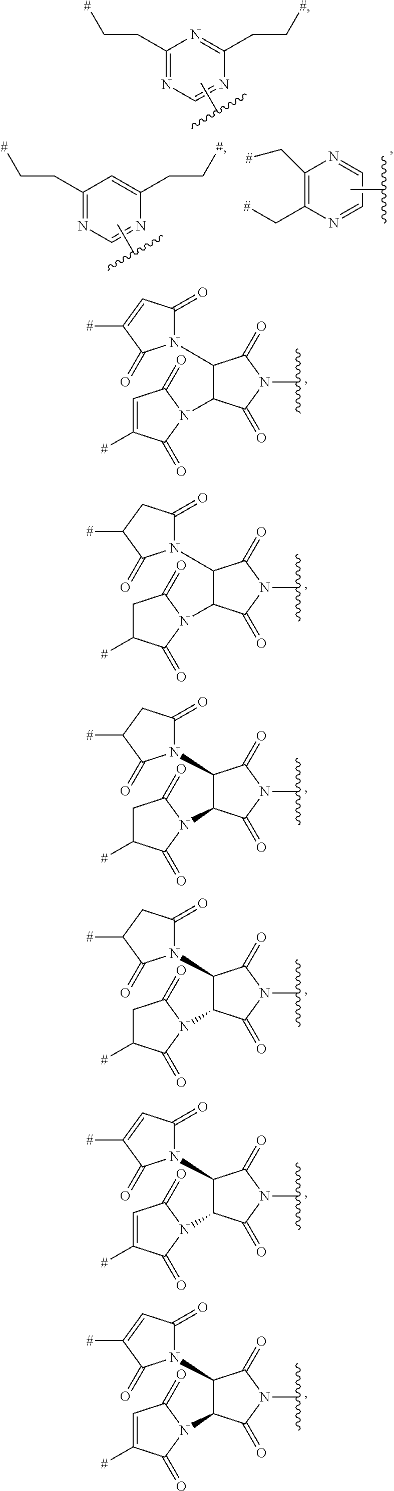

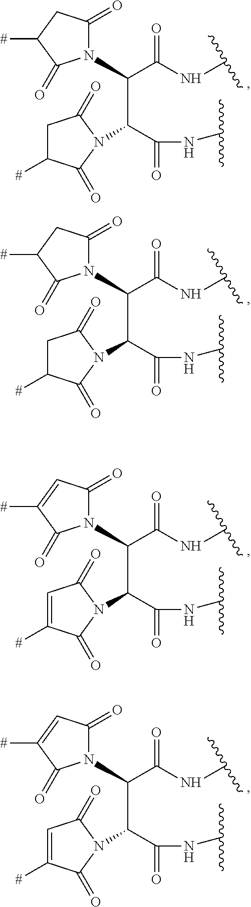

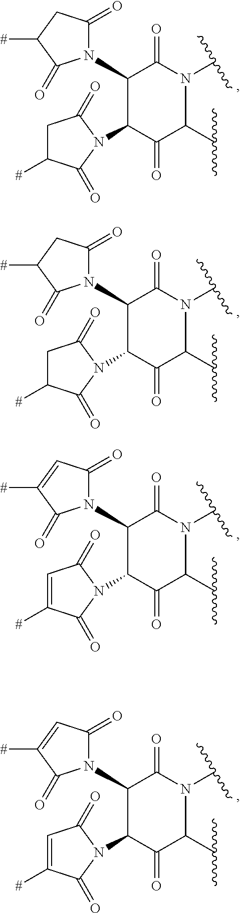

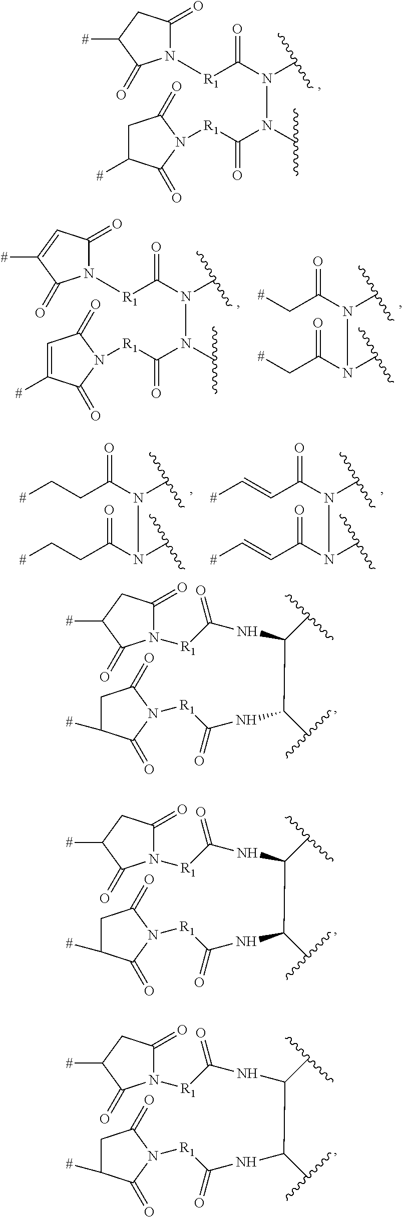

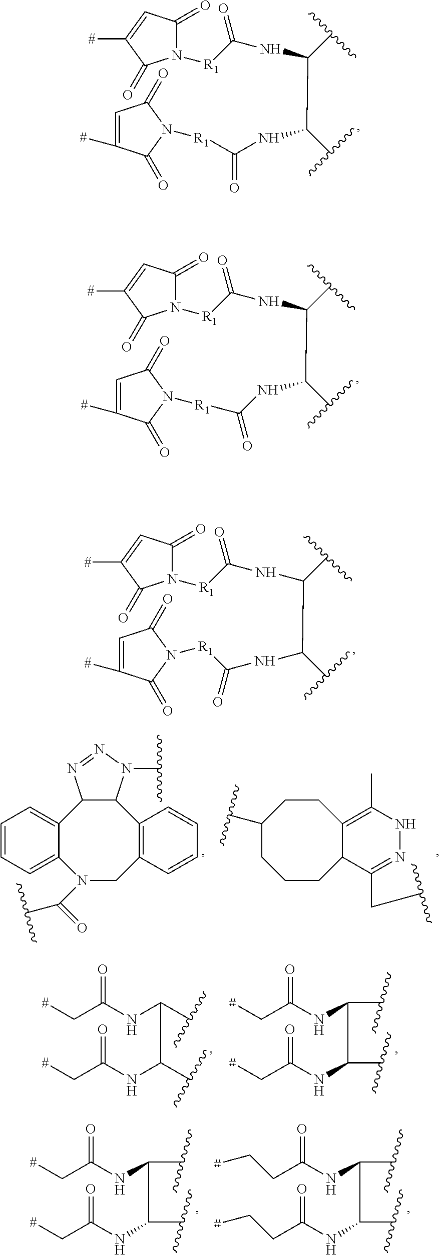

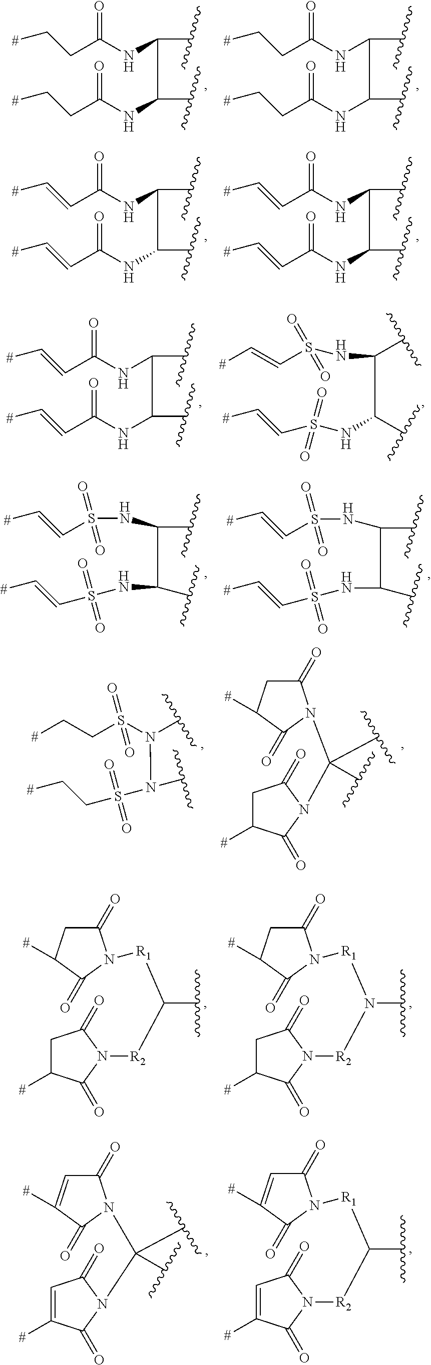

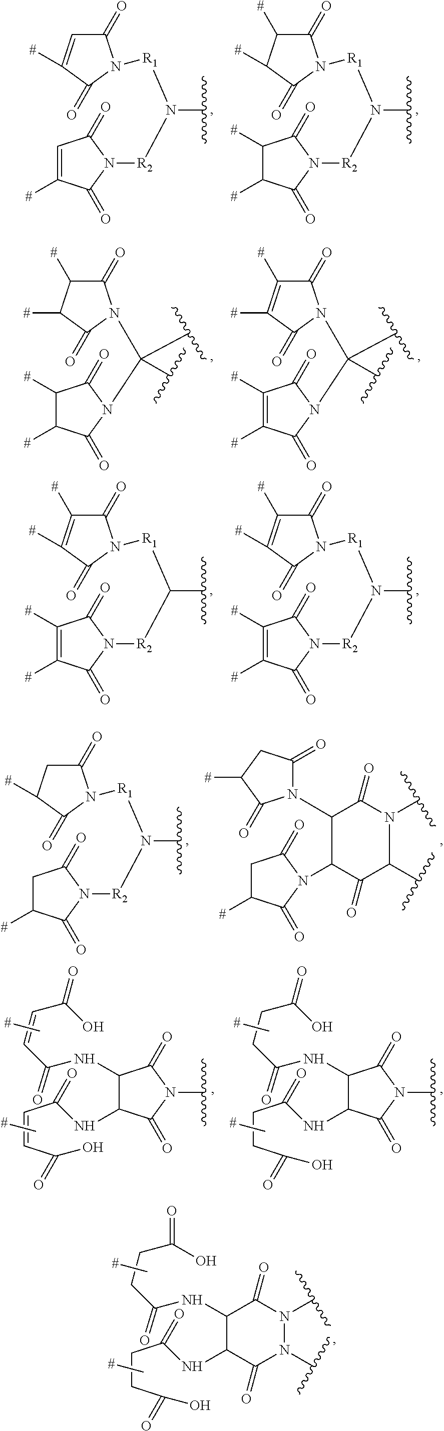

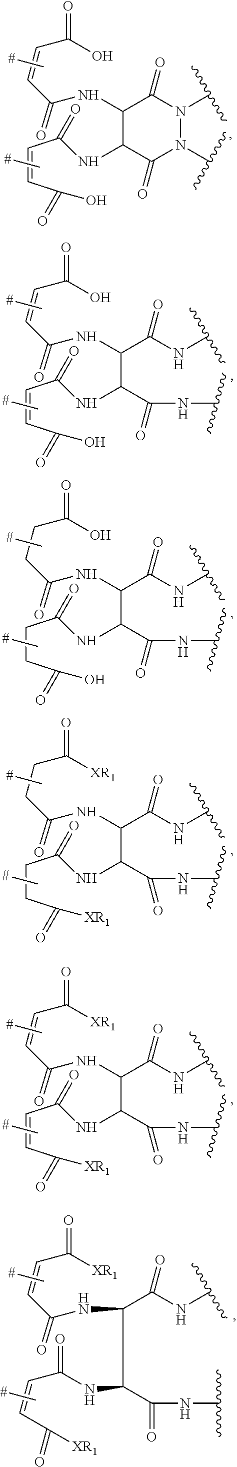

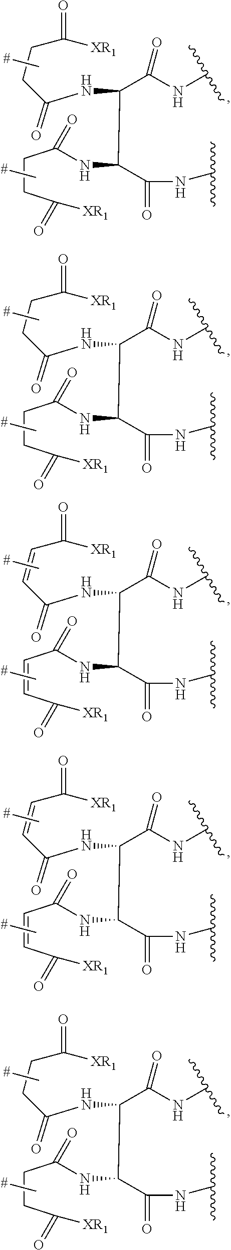

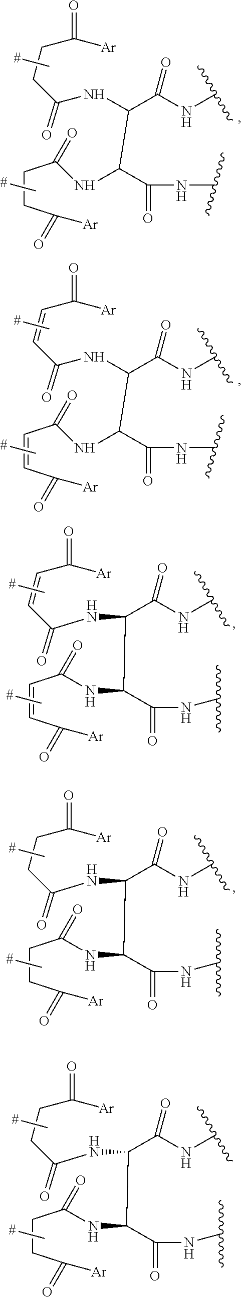

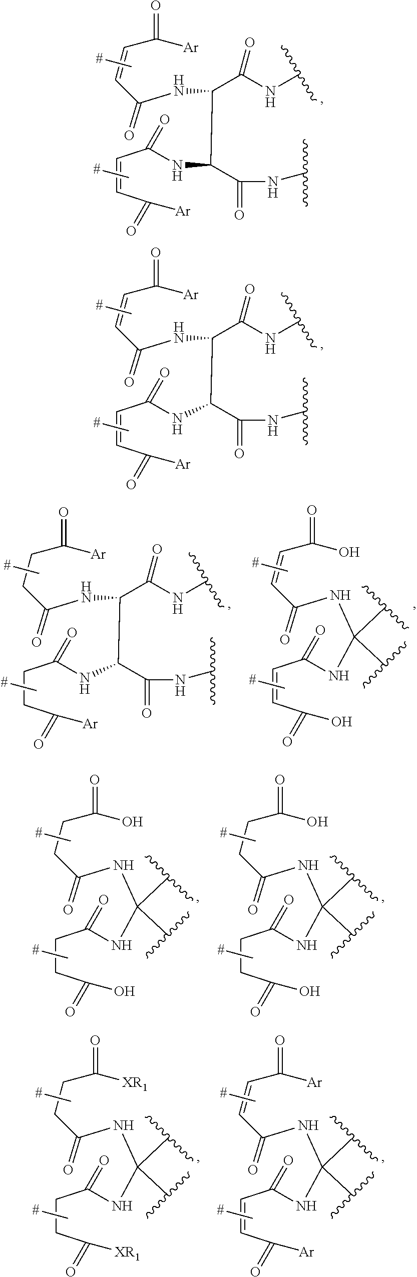

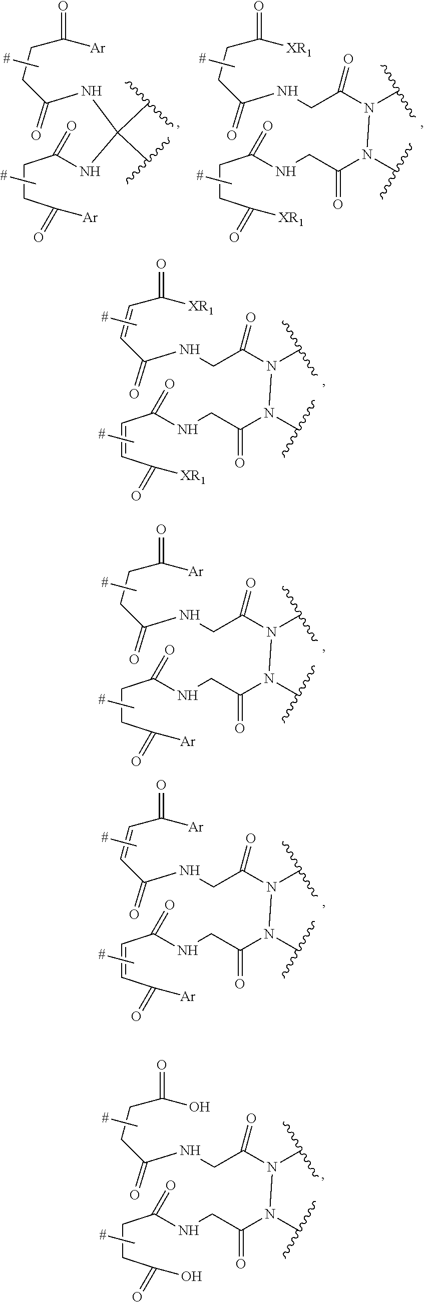

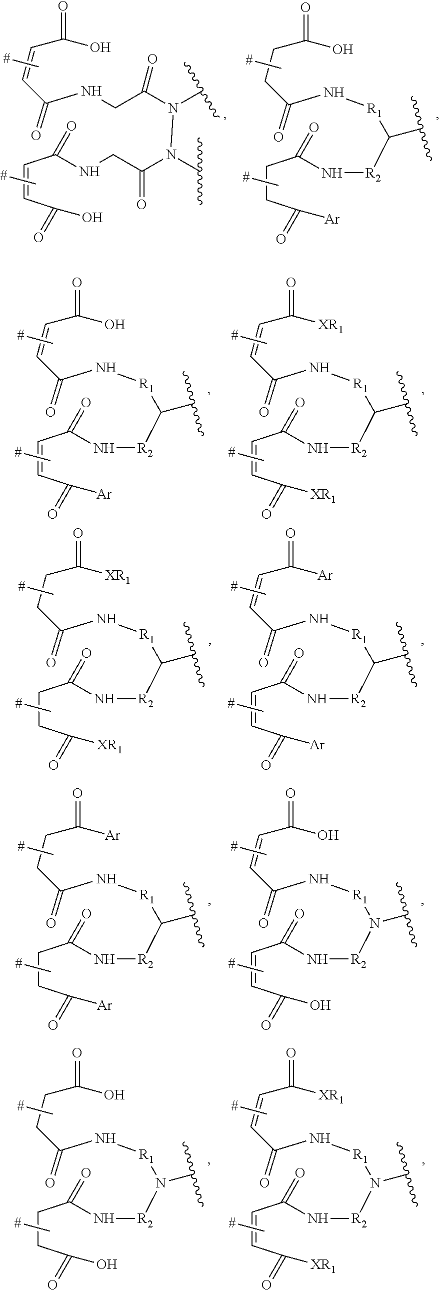

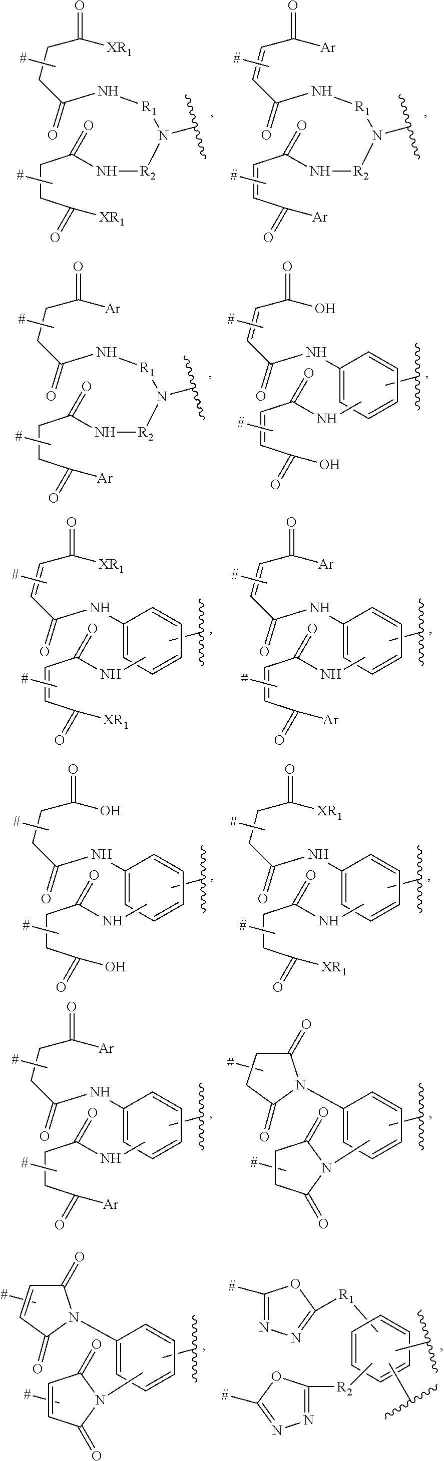

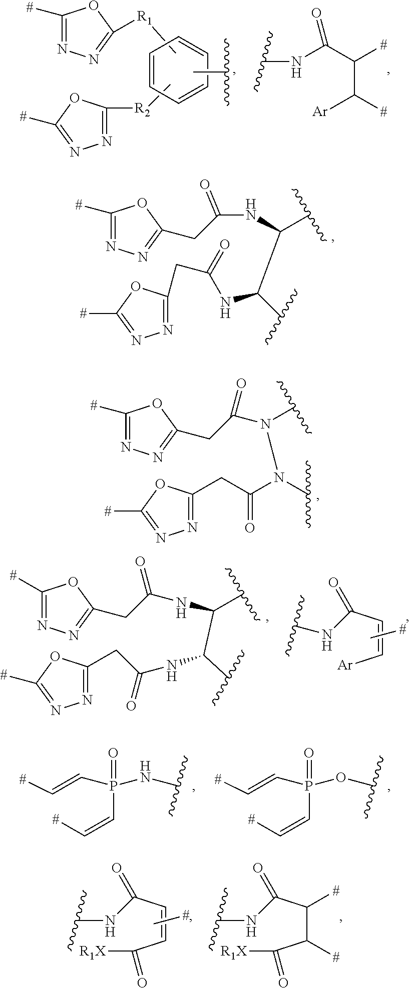

wherein “ [symbol] ” is a site that links to the linker component; “#” is the site as Lv₁′ and Lv₂′ indicated in the formulas that links a S (thiol), O (phenol), NH (amino), CHO (aldehyde), C(═O) (ketone), C(O)(NH) (amide) and C(O)(OH) (carboxylate) of an antibody; wherein R₁, X₁′ and X₂′ are described above; X is O, NH, S, CH₂; the conneting bond “—” in the middle of the two atoms means it can link either one of the two atoms, Ar is an aromatic group.

More preferably, the following core linker structure (L₁′) having an affinity ligand in Formula (I) (or L₁″ in Formula (Ib′)) as:

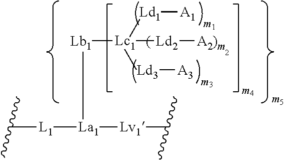

is preferably selected from:

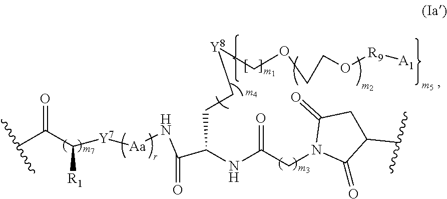

Wherein Aa is L-or D- natural or unnatural amino acids; A₁ is the affinity ligand defined the same above;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0-12; when r is not 0, (Aa)ᵣ is the same or different amino acids or peptide units;
m₁=1-18; m₂=1-100; m₃=1-8; m₄=0-8; m₅=1-6; m₇=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H4NH, CH₂;
Y⁸ is NHC(═O), NH, O, NHS(O₂), NH(SO), NHS(O₂)NH, NHP(O)(OH)NH,

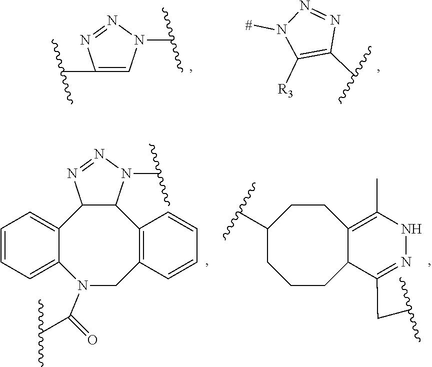

### C(O)O, C(O), OC(O)NH, C(O)NH, or Ar;

R₉ is (O═)CR₁, (O═)CNHR¹, NHC(═O), NH, O, NHS(O₂), NH(SO), NHS(O₂)NH, NHP(O)(OH)NH or C(O)NH, R₁(COCH₂NH)ₘ₄H, R₁(Aa)ᵣ, (Aa)ᵣ, C(O), Ar or

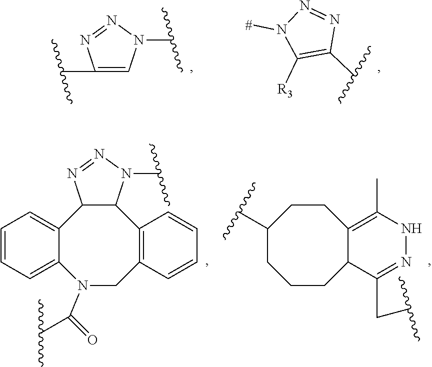

wherein R₃ is H, C₁-C₈ alkyl, ester, amide, Ar, ketone, alkyl acid, alkyl alcohol, alkyl amine, CH₂C₆H₅, CH₂C₆H₄OH, CH(OH)CH₃, CH₂C(O)NH₂, CH₂CH₂C(O)NH₂, CH₂CH₂CH₂NHC(—NH)NH₂; R₁ is defined above.

Or the following core structure (called L₁″ and L₂″ fused jointly) in Formula (III) (or the L₁″ and L₂″ fused in Formula (IIb′) accordingly):

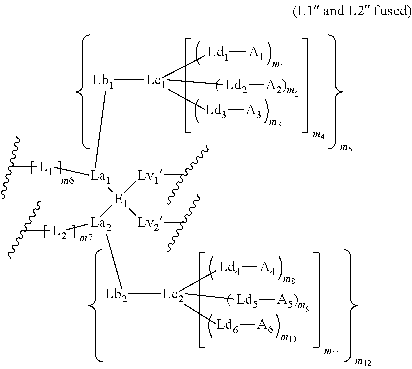

is preferably the following formula (Ib) and (Ic):

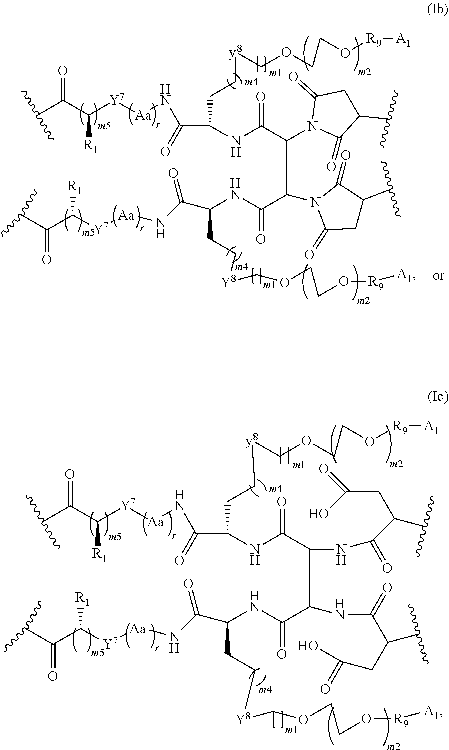

Wherein R₁, Y⁷, Y⁸, R₉, A₁, Aa, r, m₁, m₂, m₄, and ms are defined the same above.

In certain embodiments, the examples of the conjugates of formula (I), (II), (III) and (IV) are illustrated below:

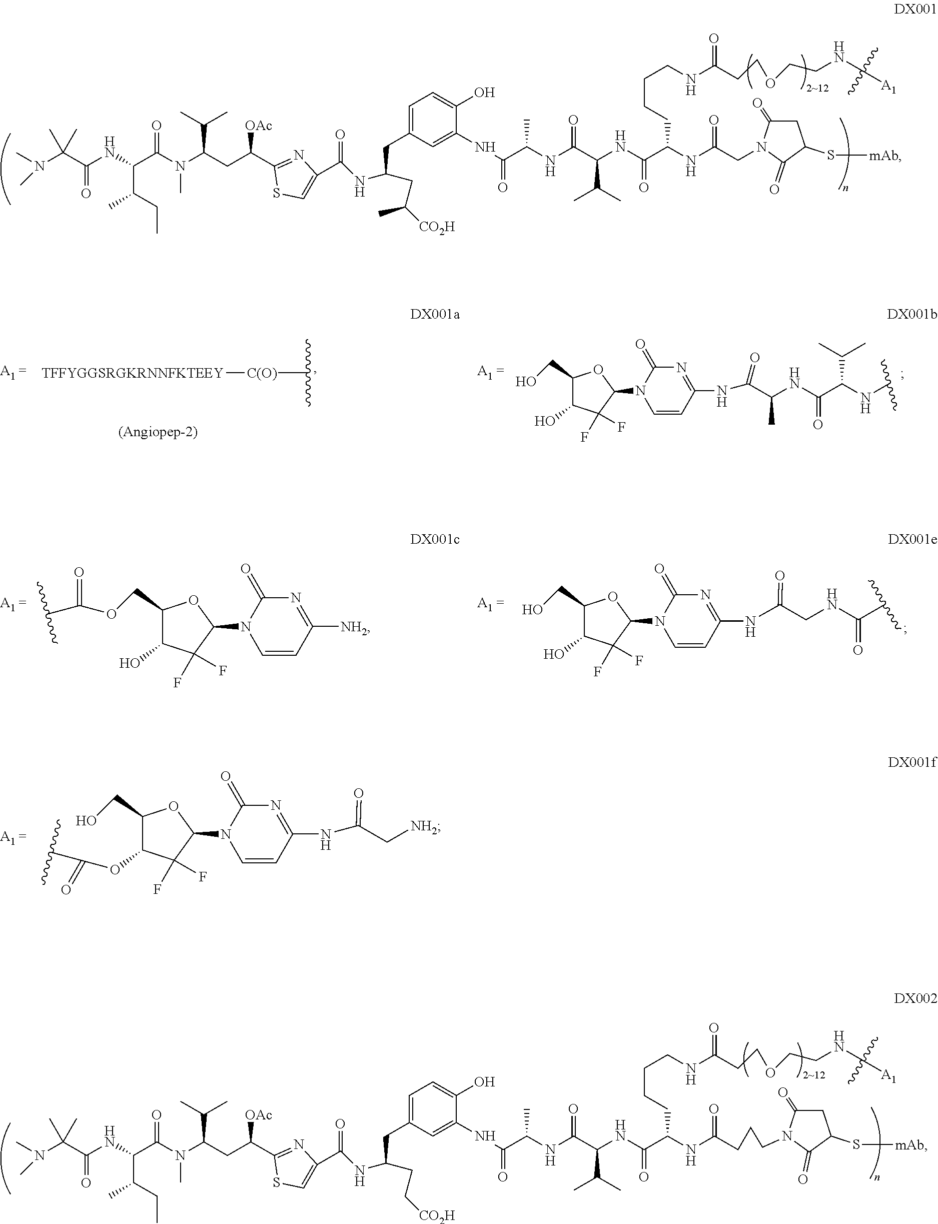

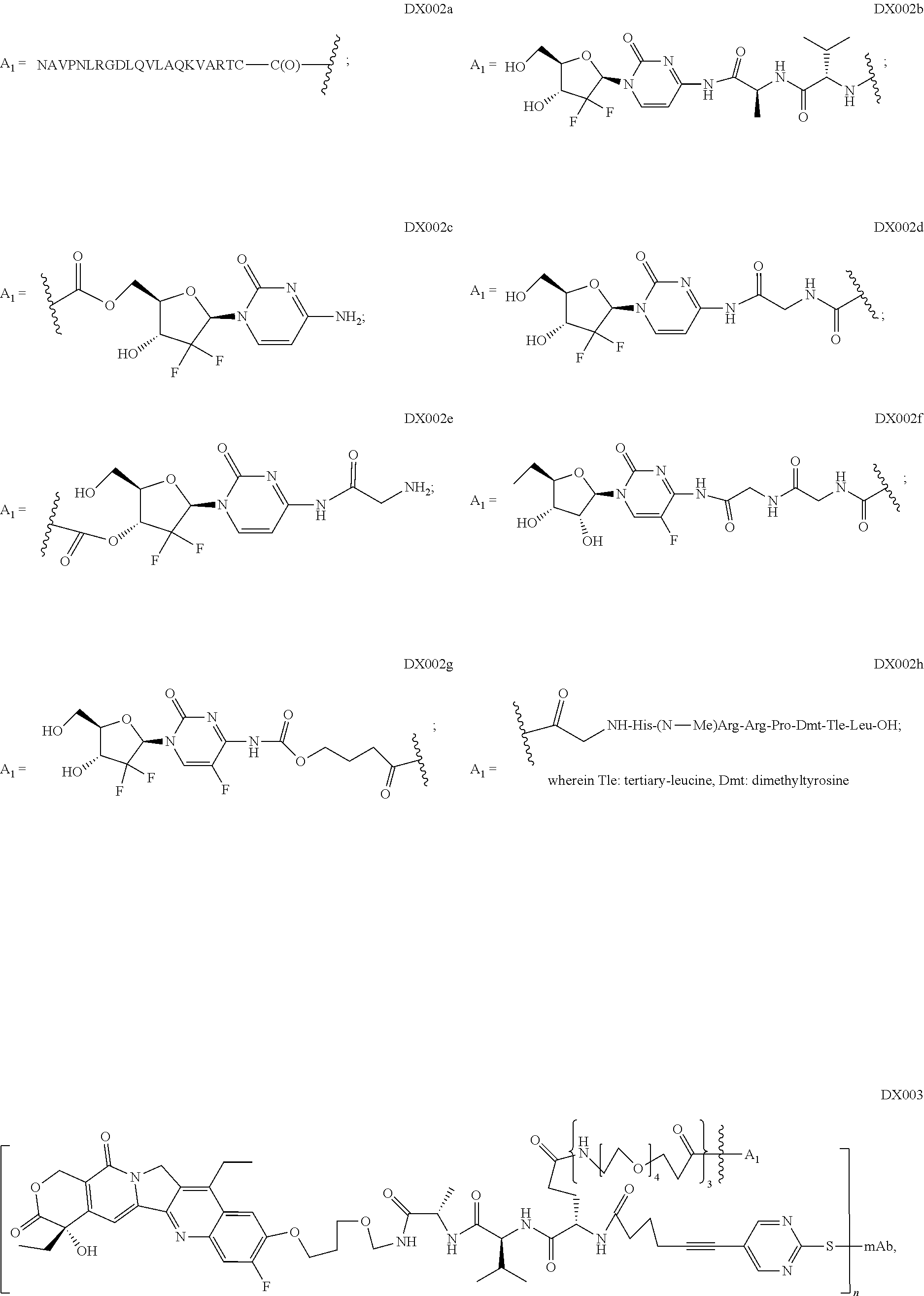

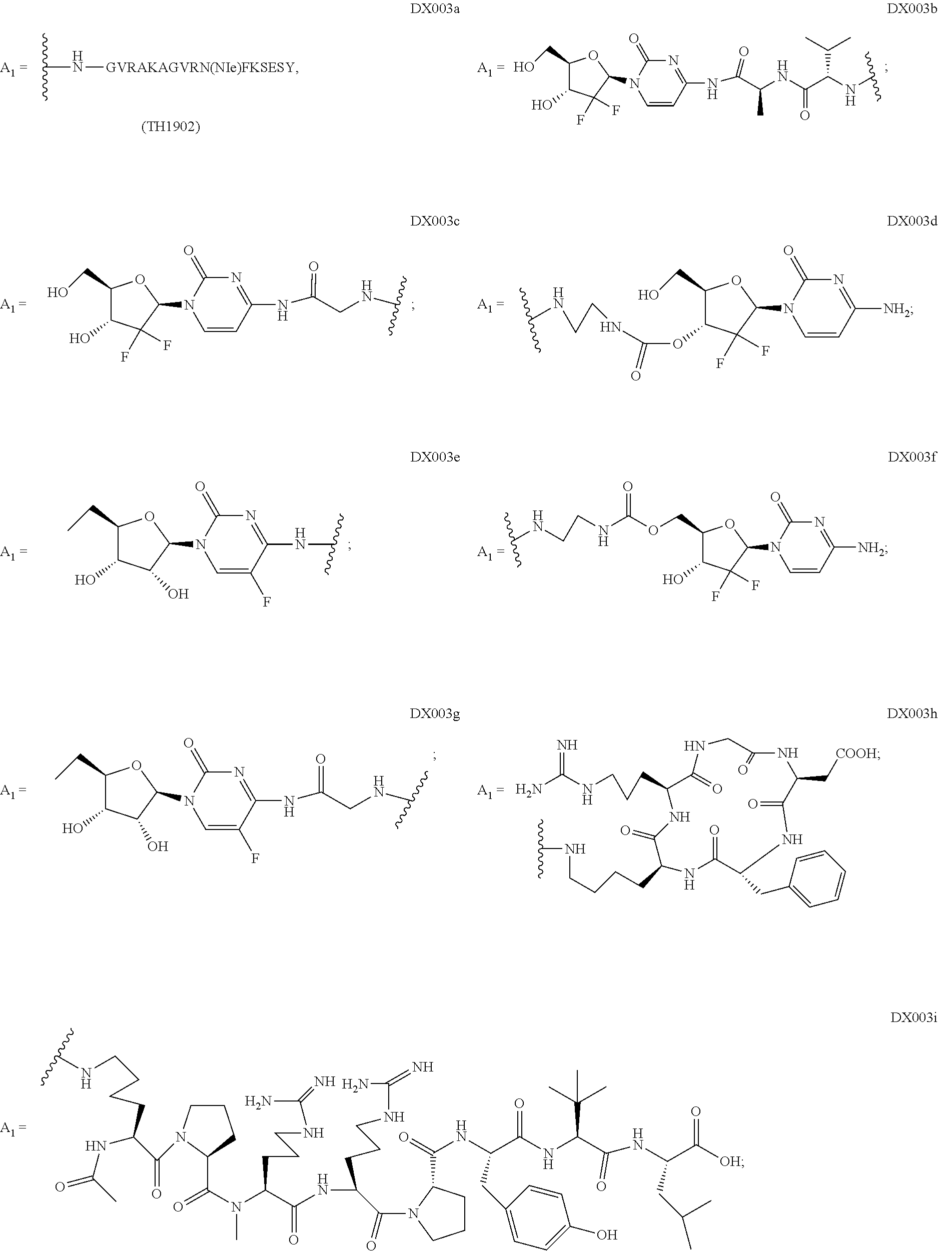

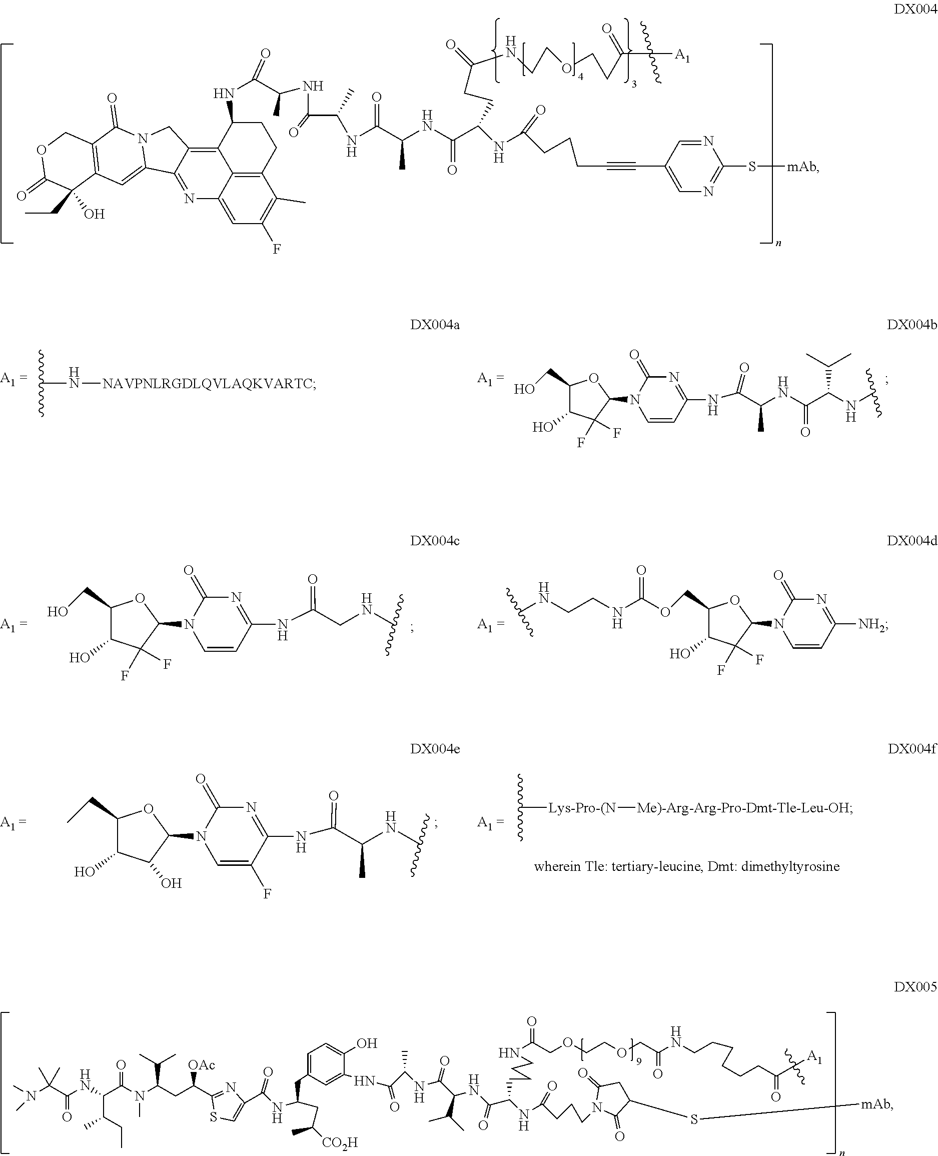

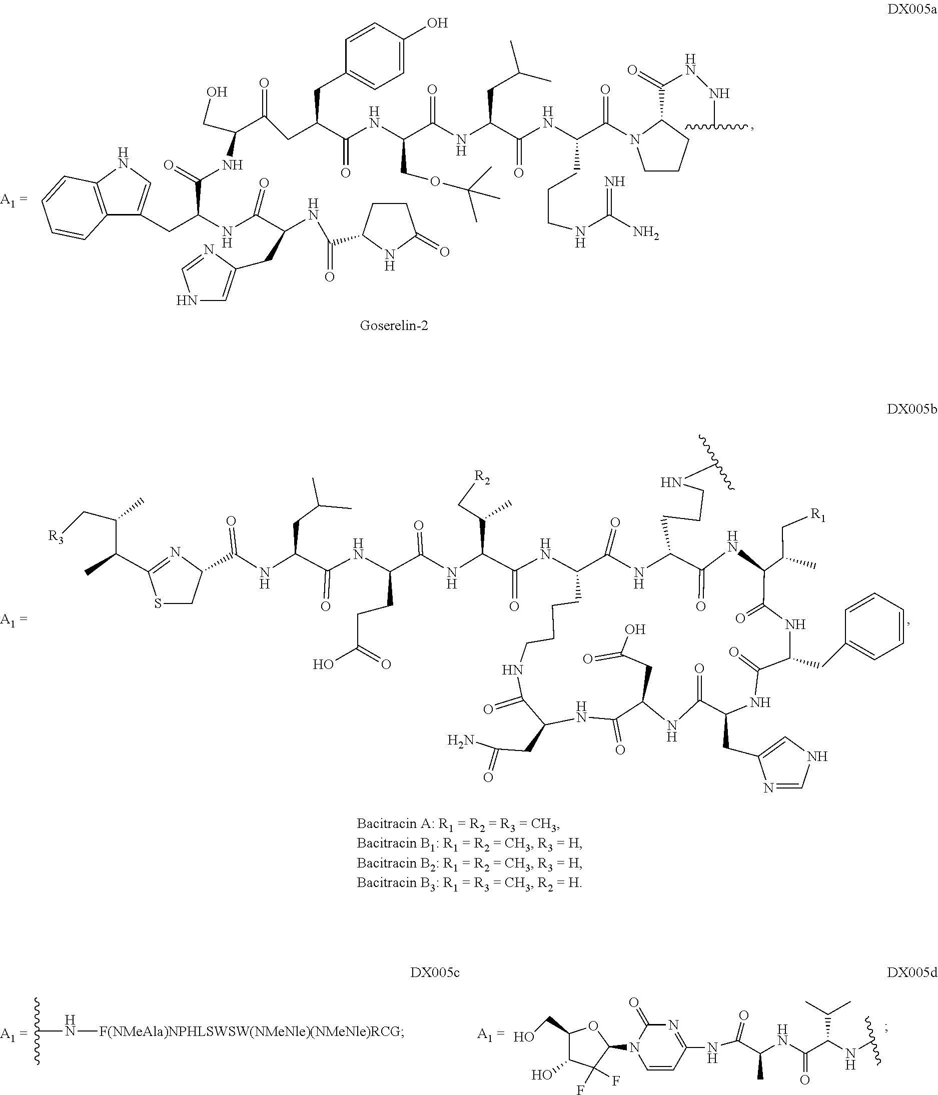

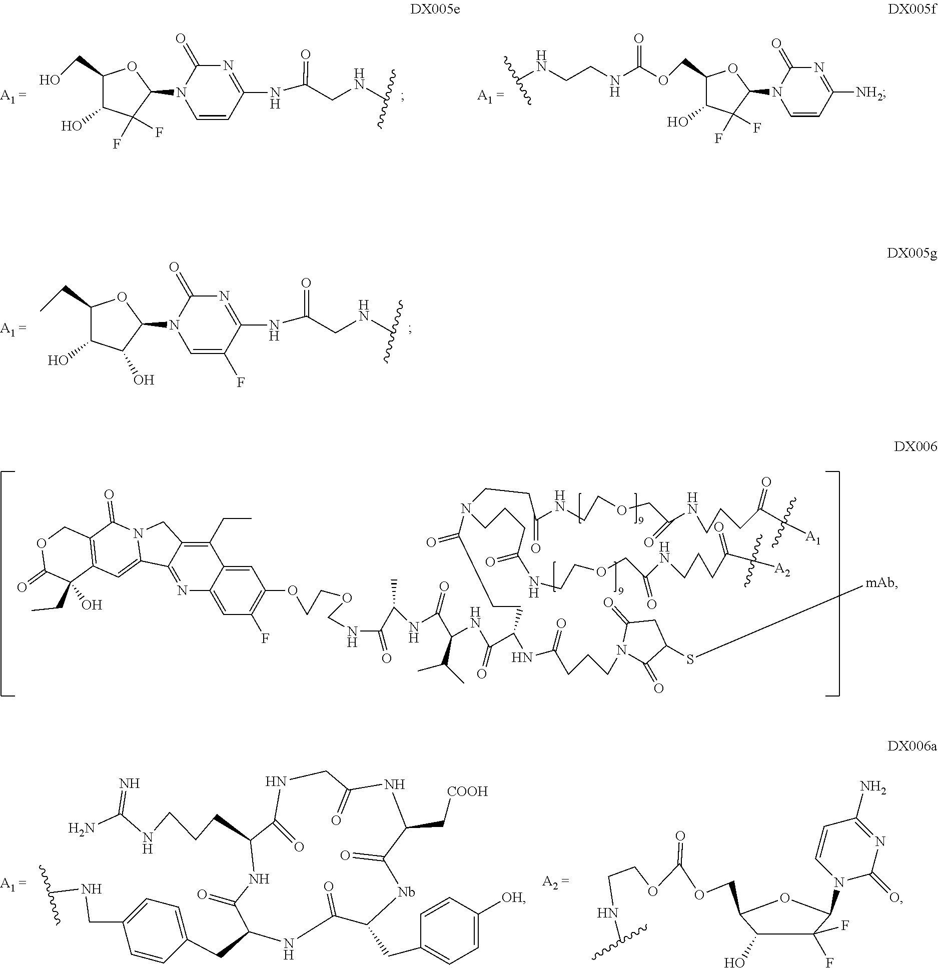

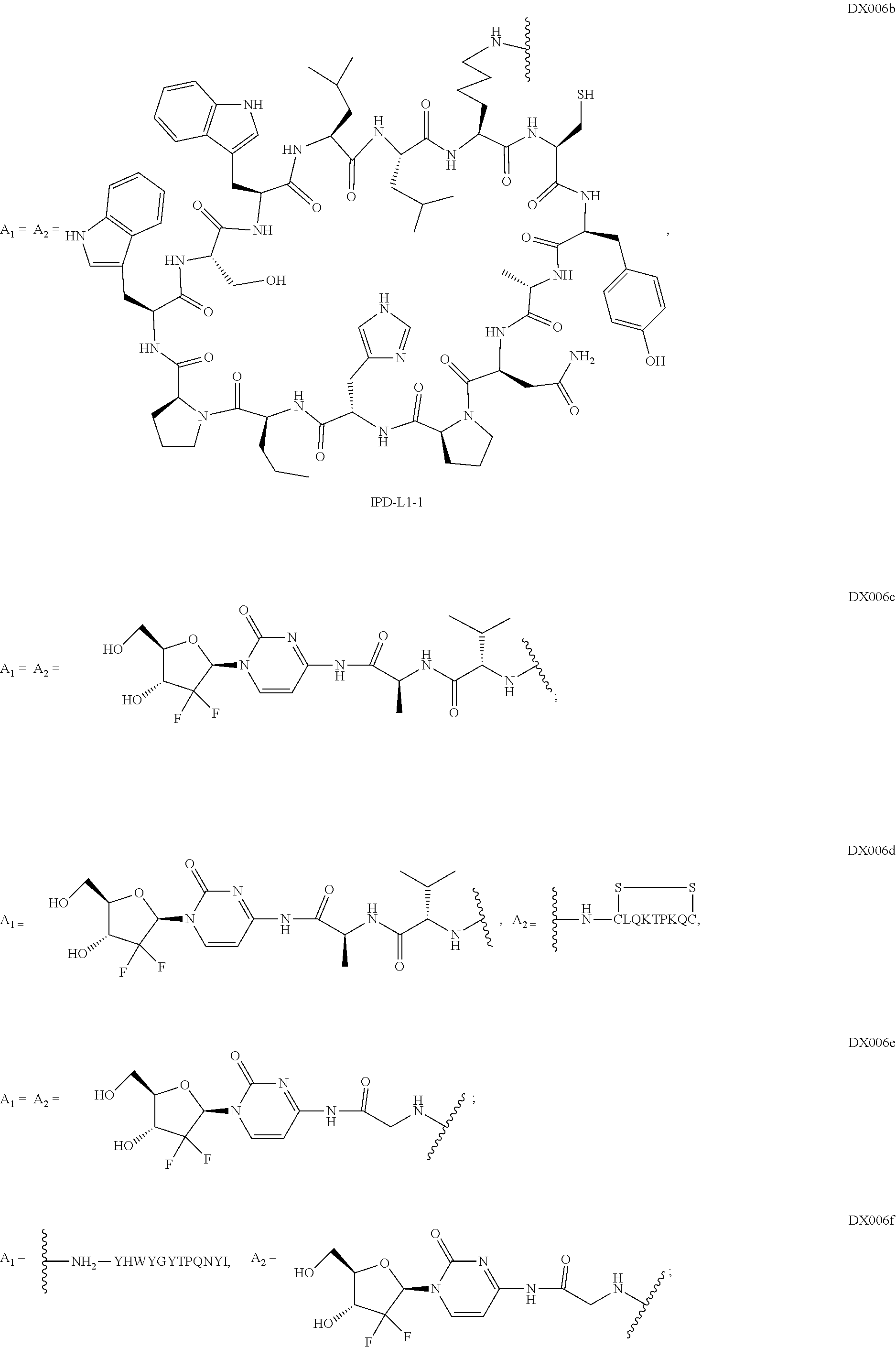

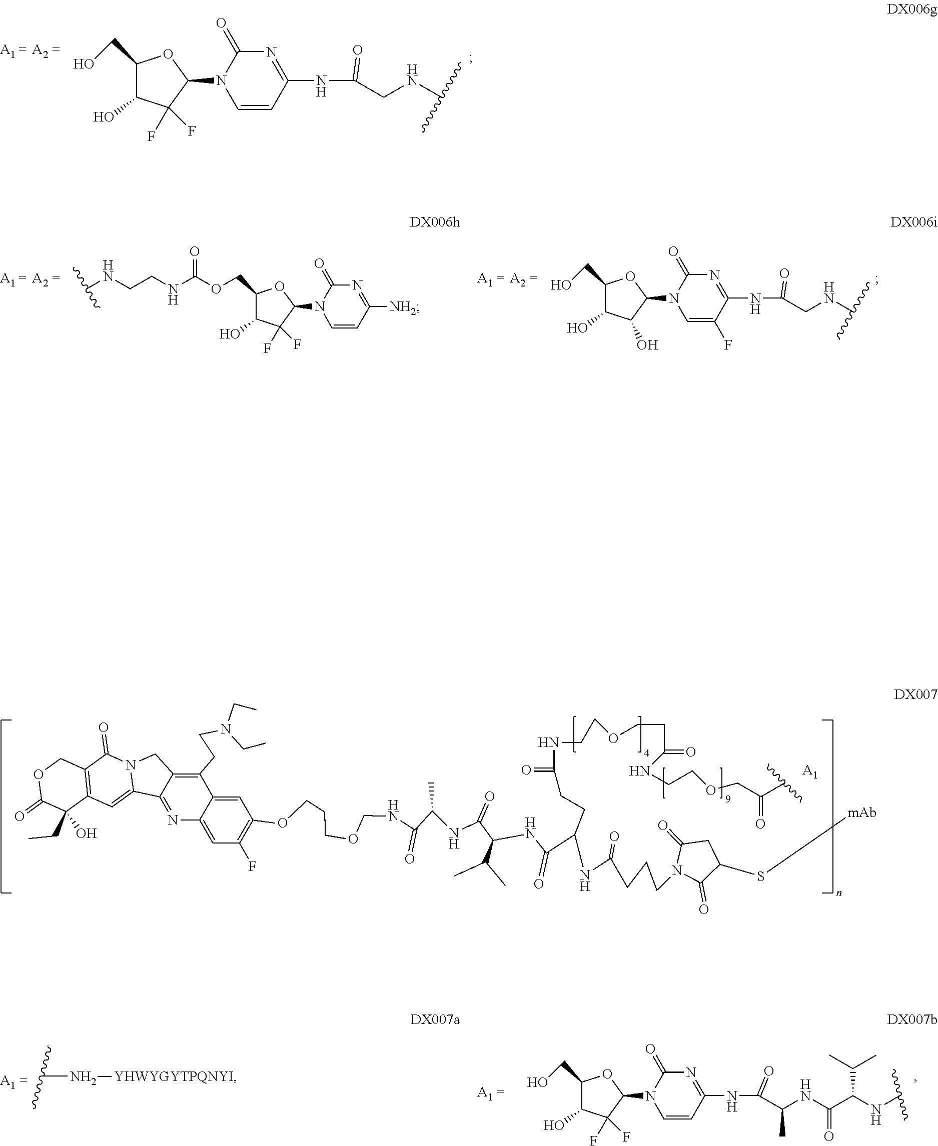

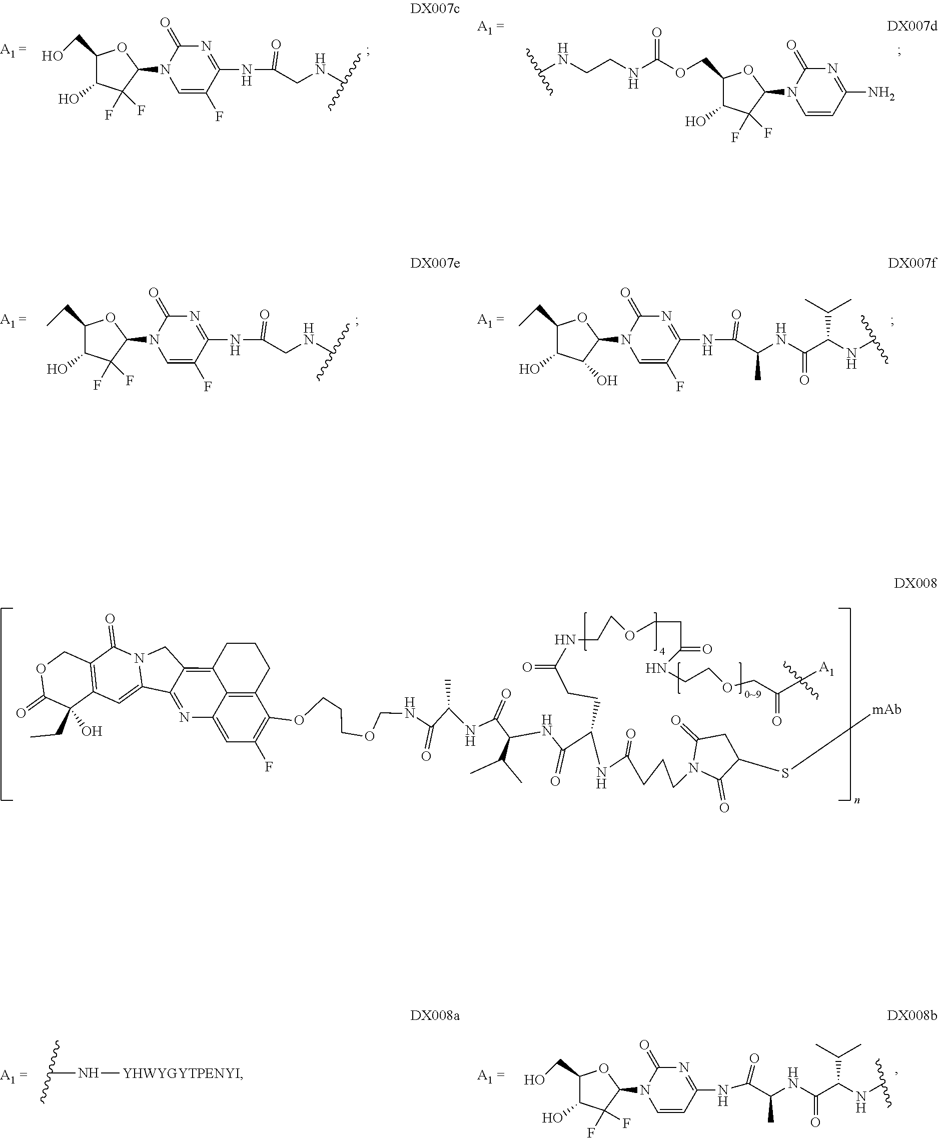

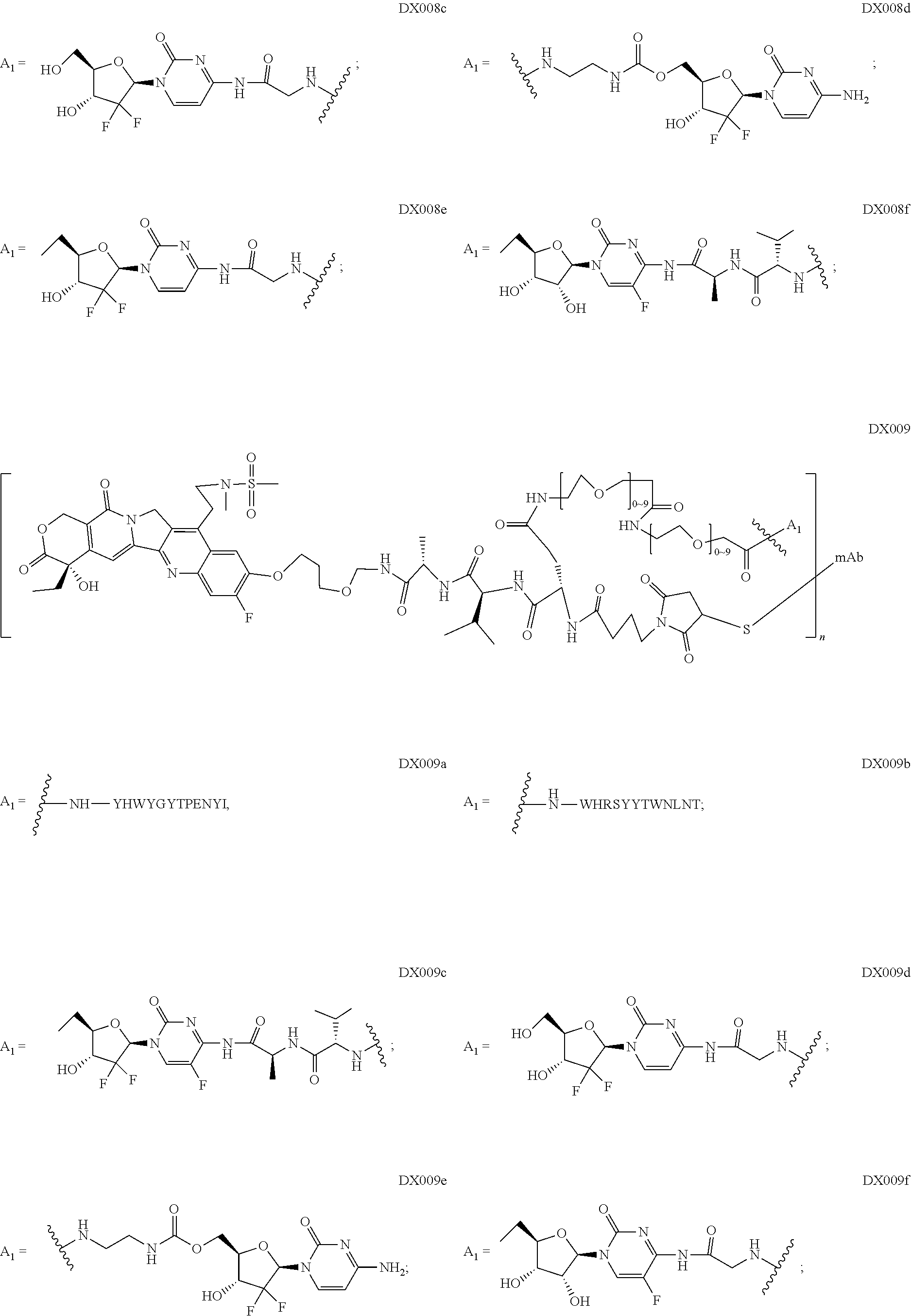

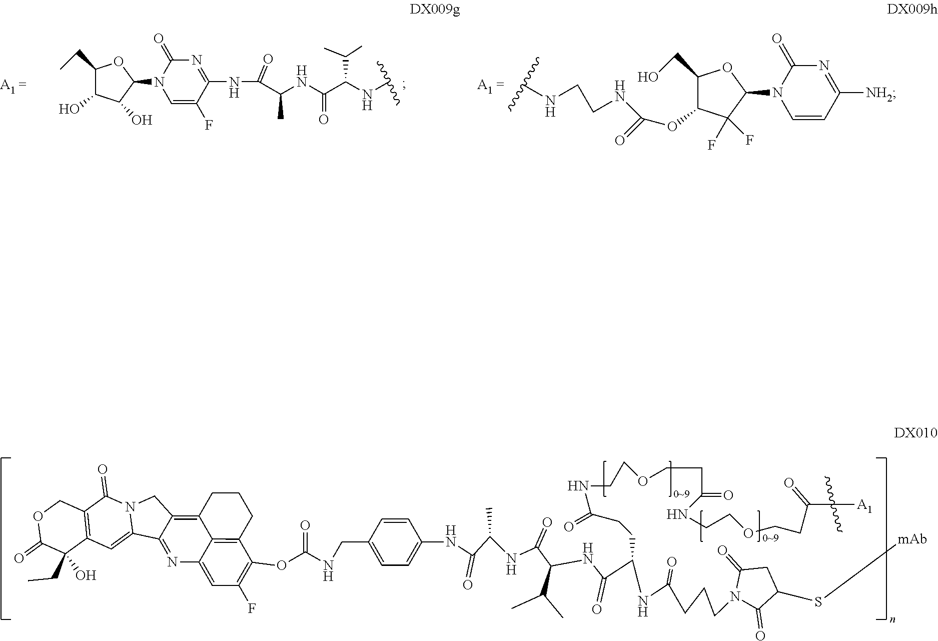

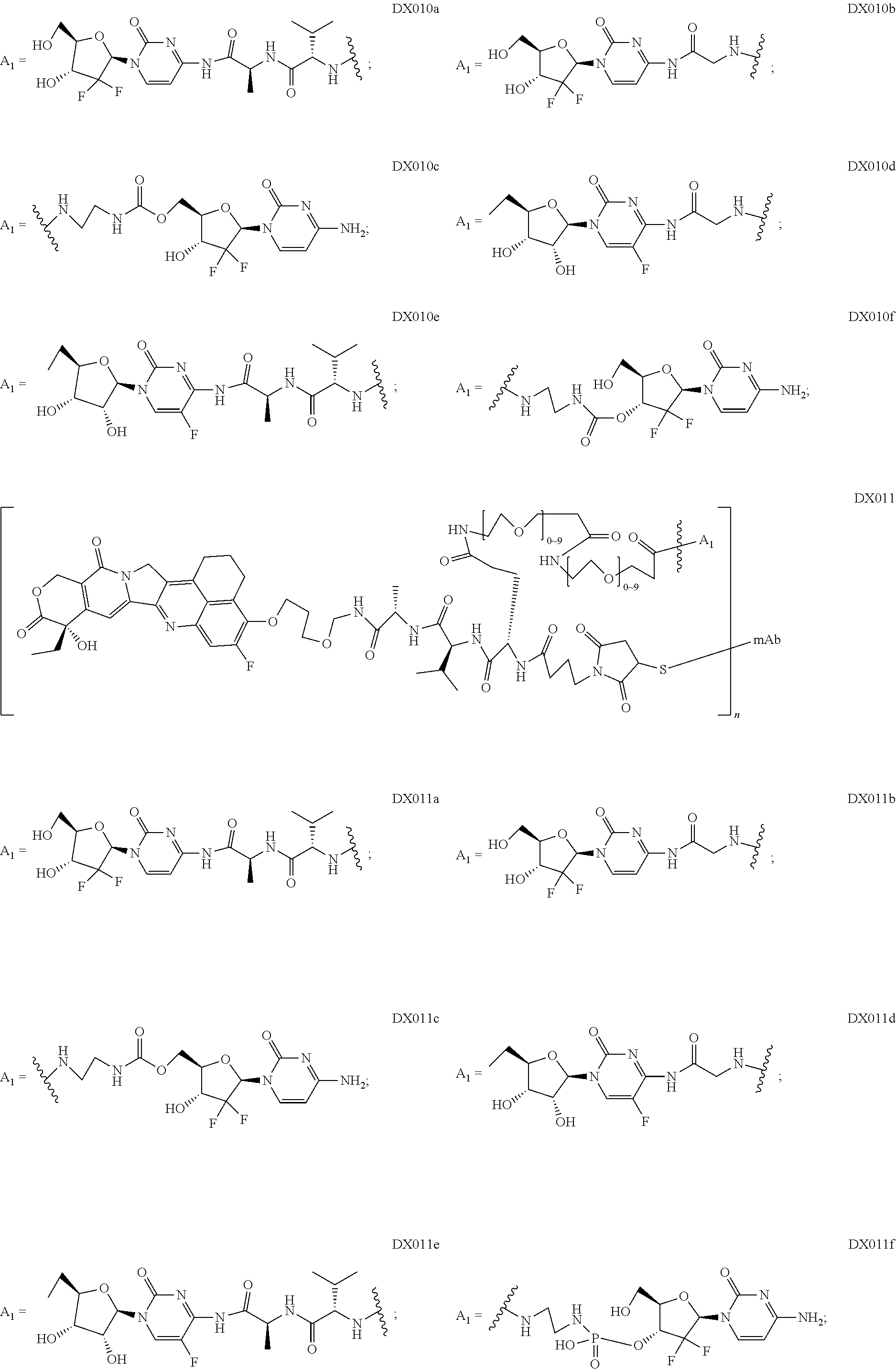

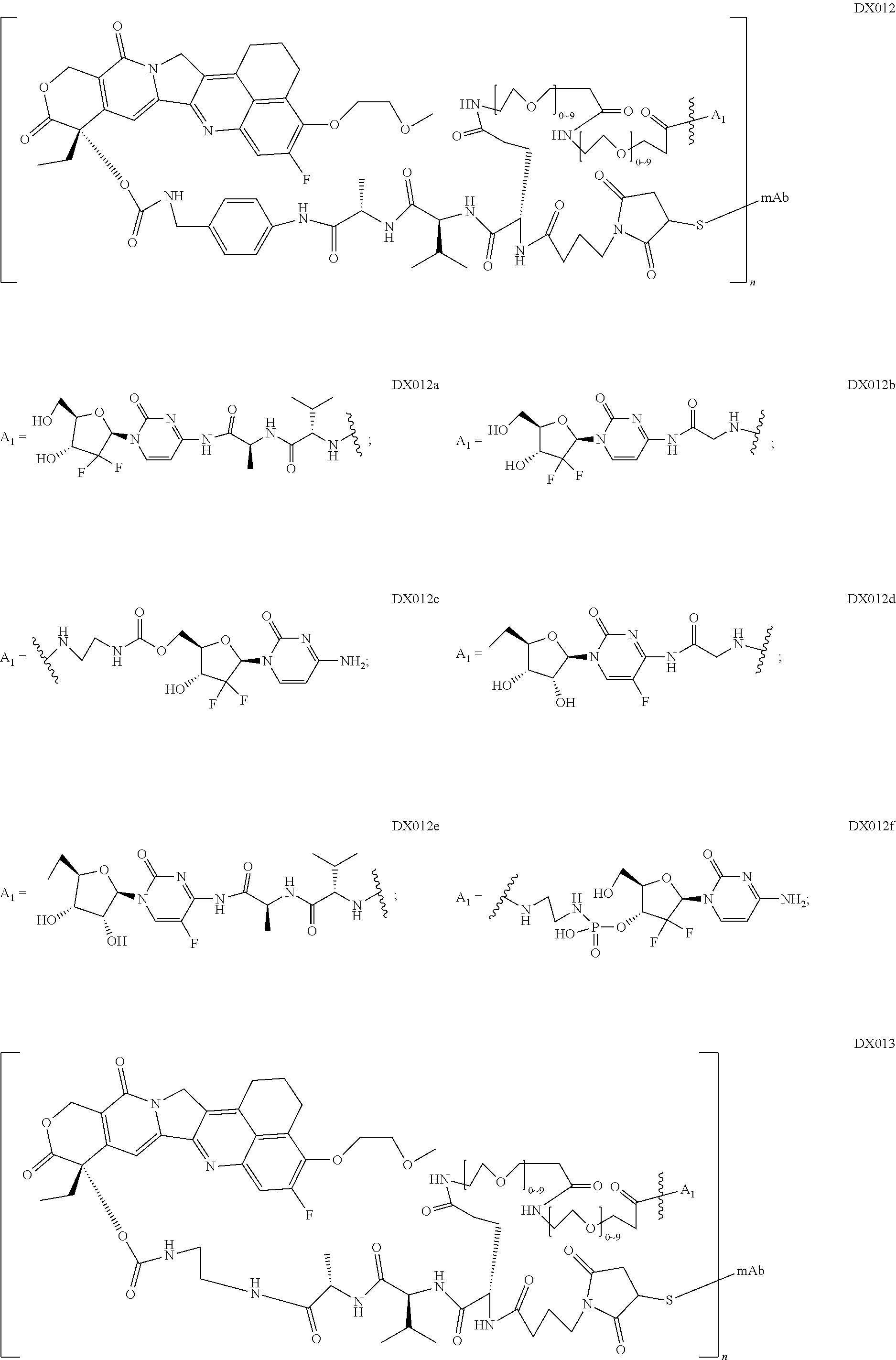

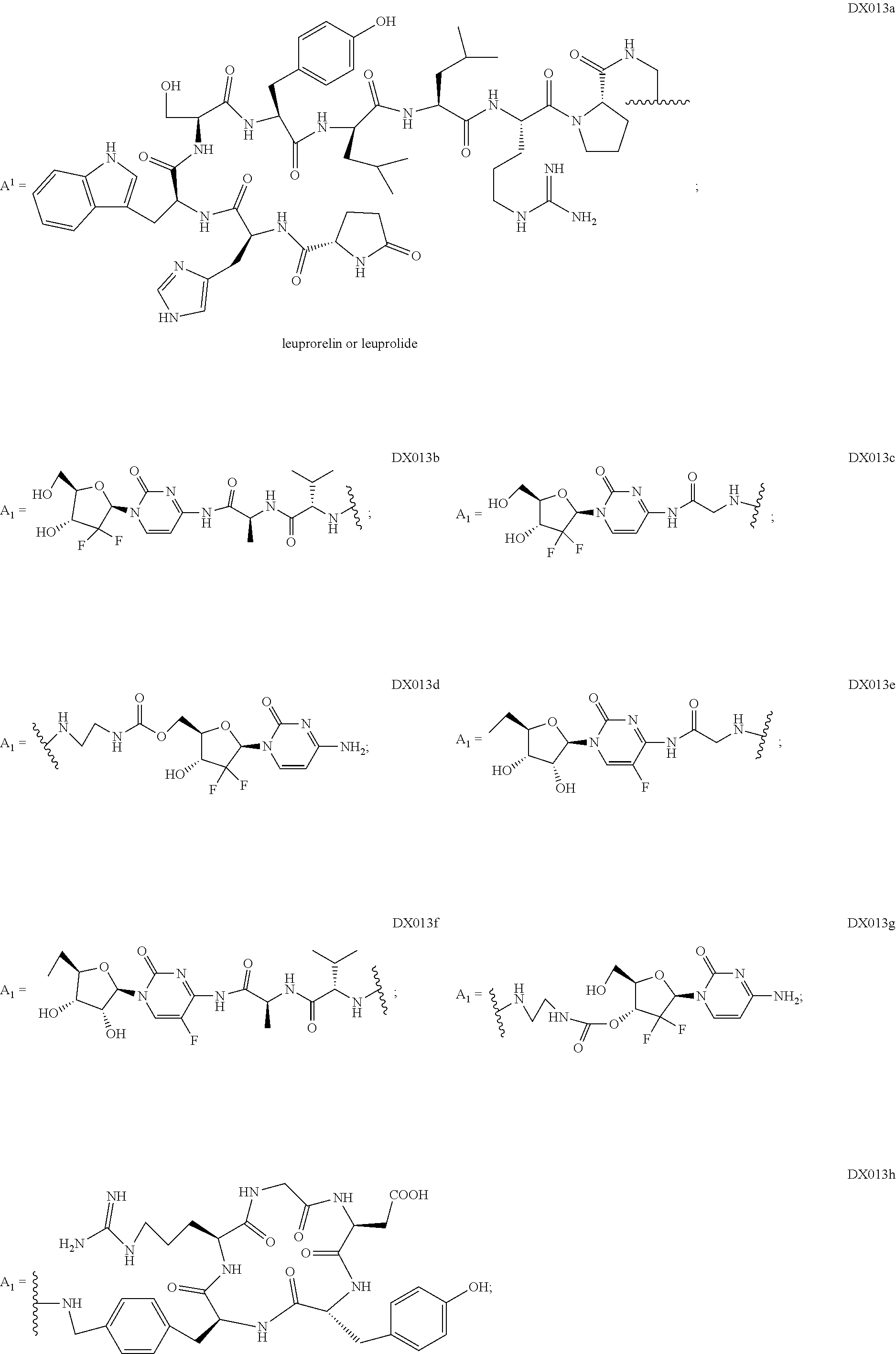

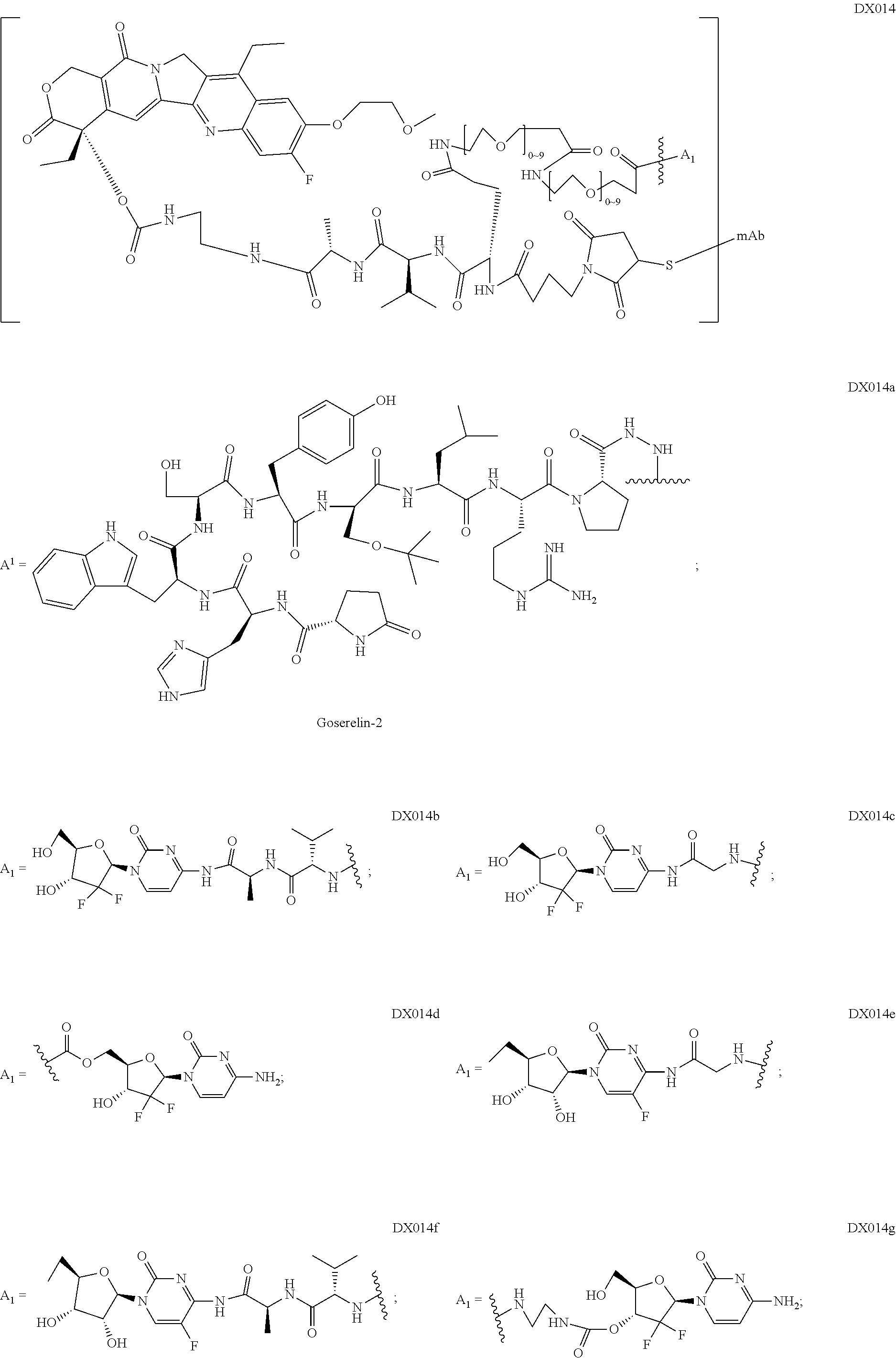

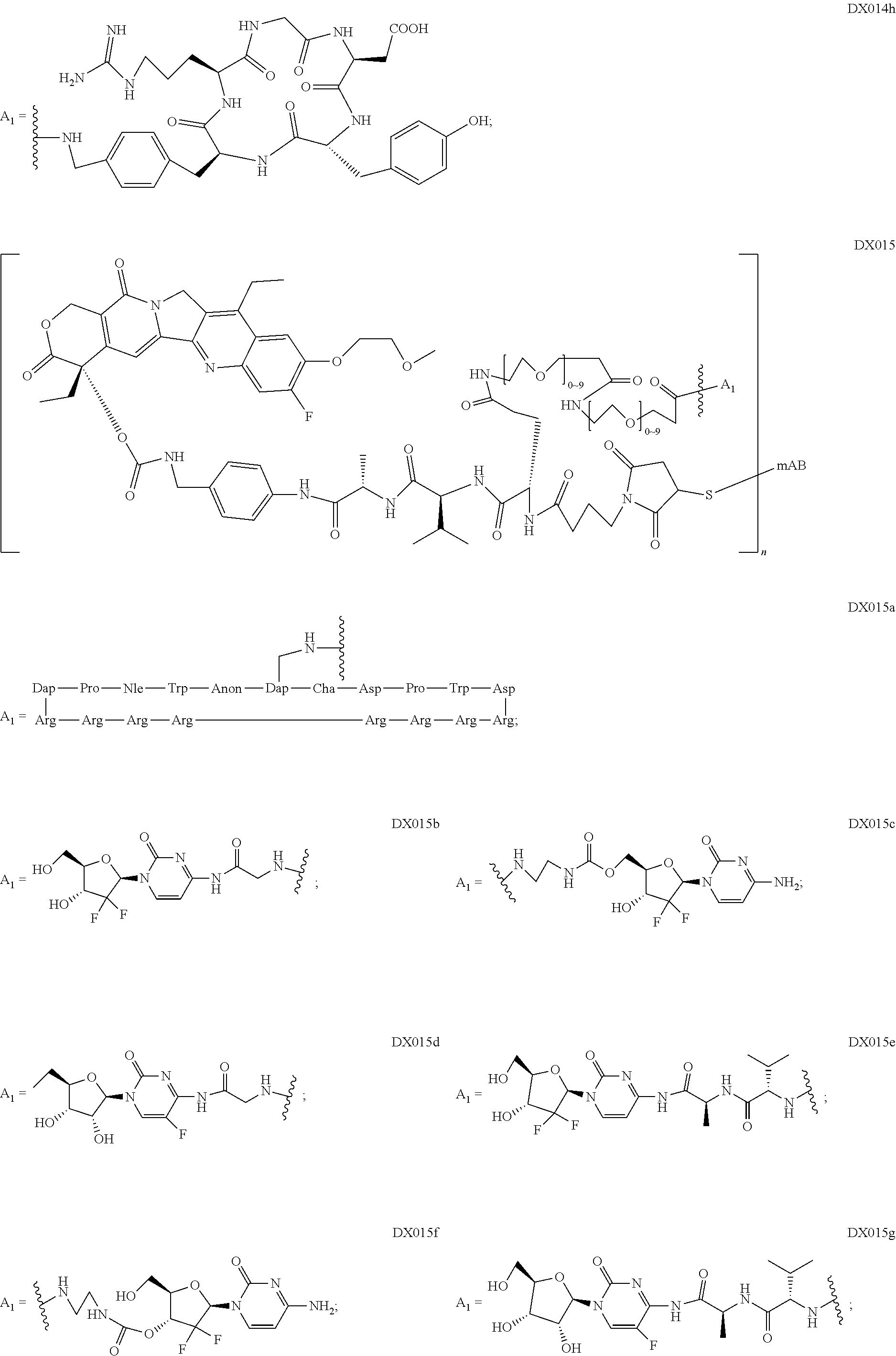

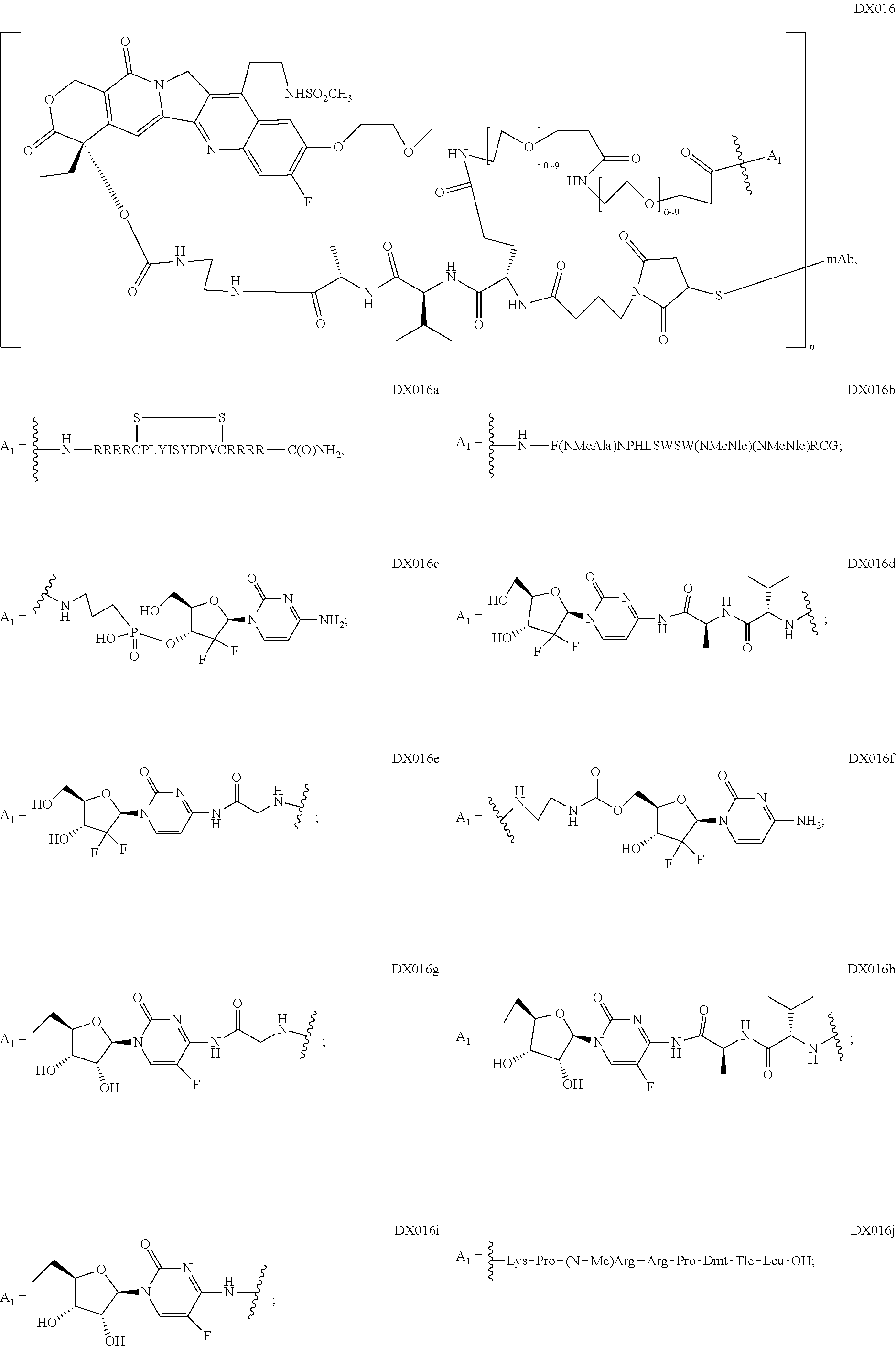

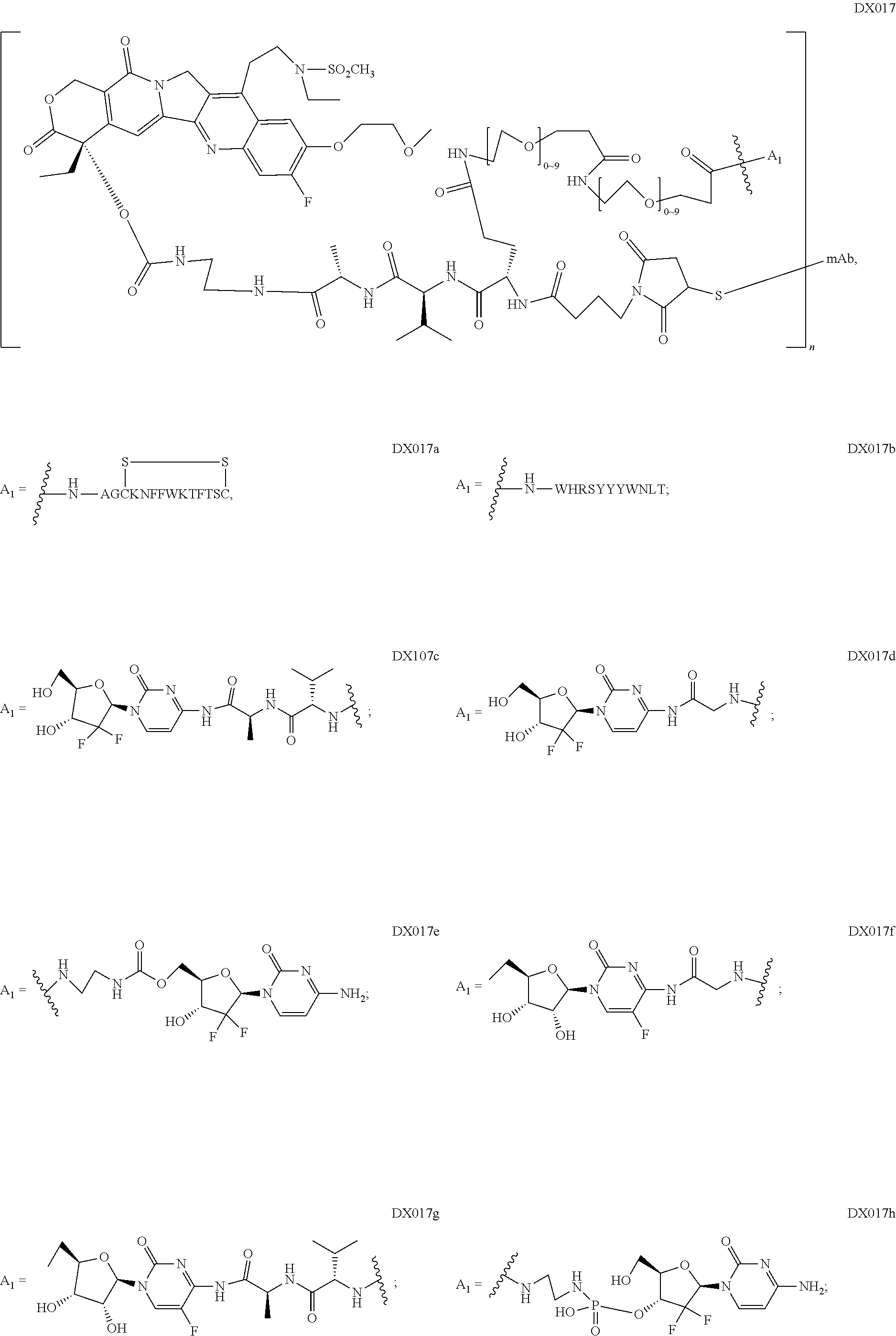

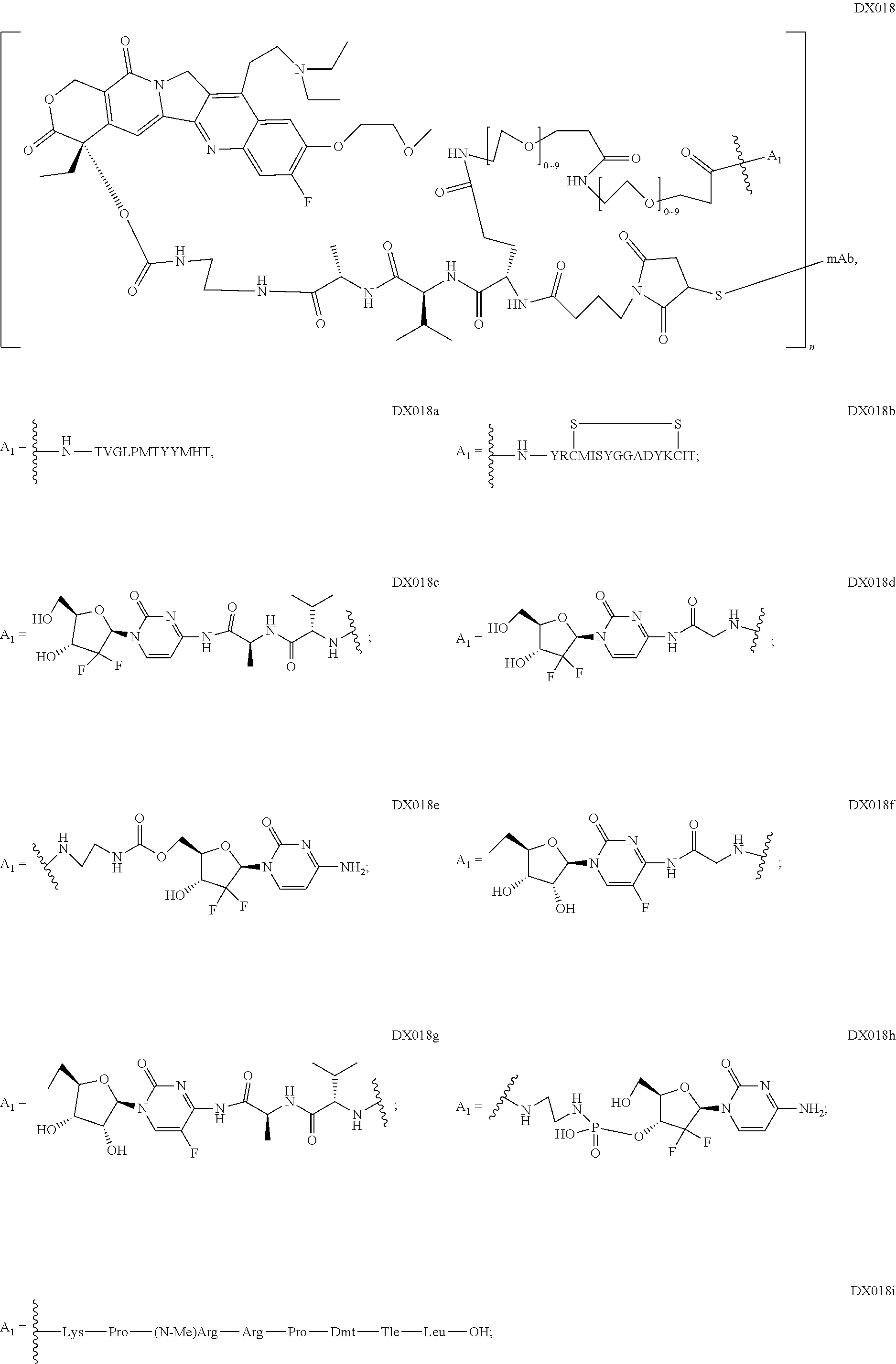

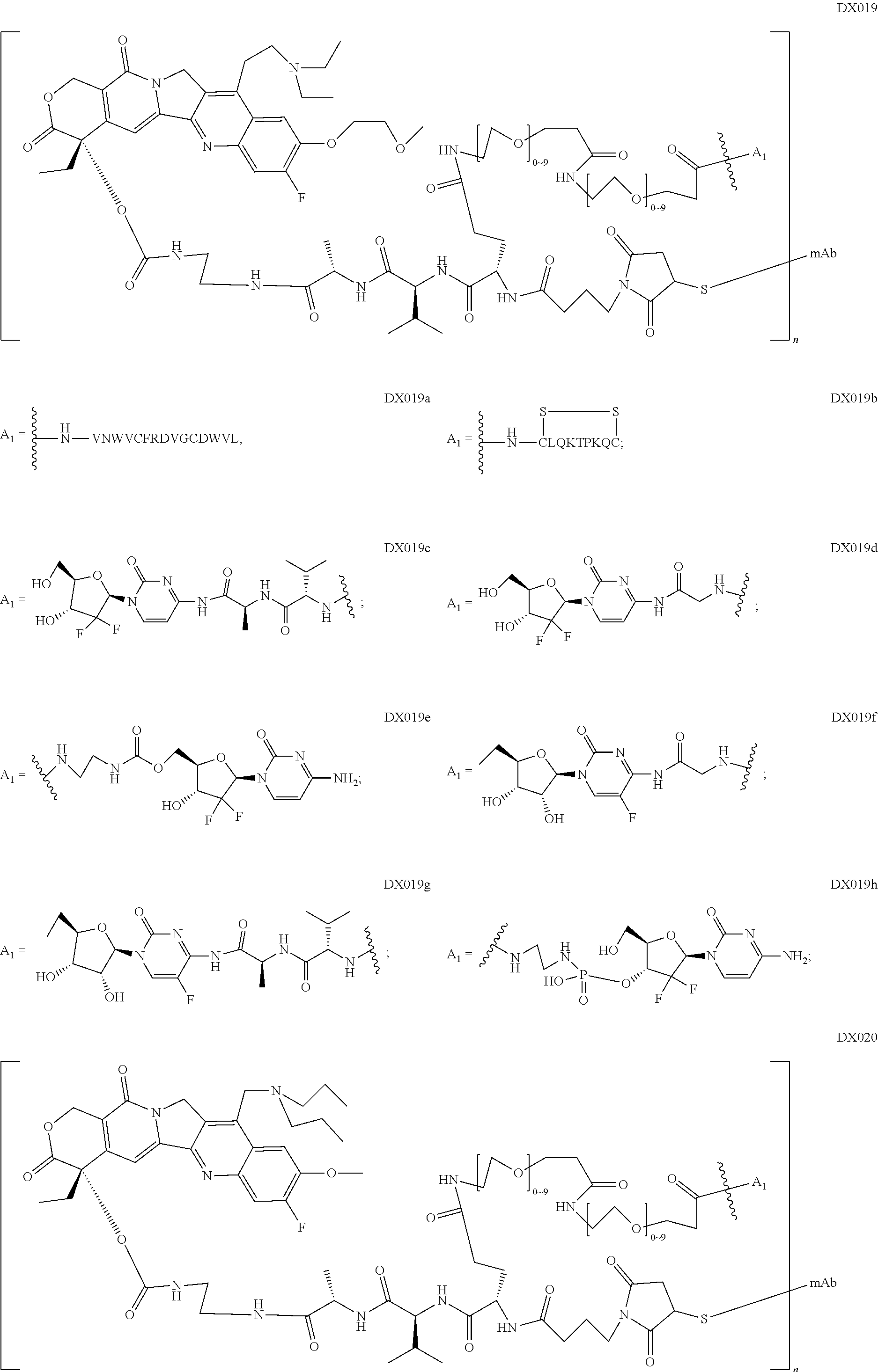

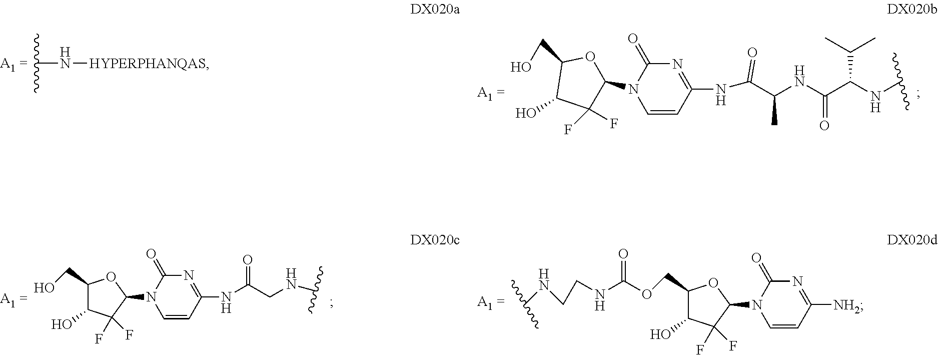

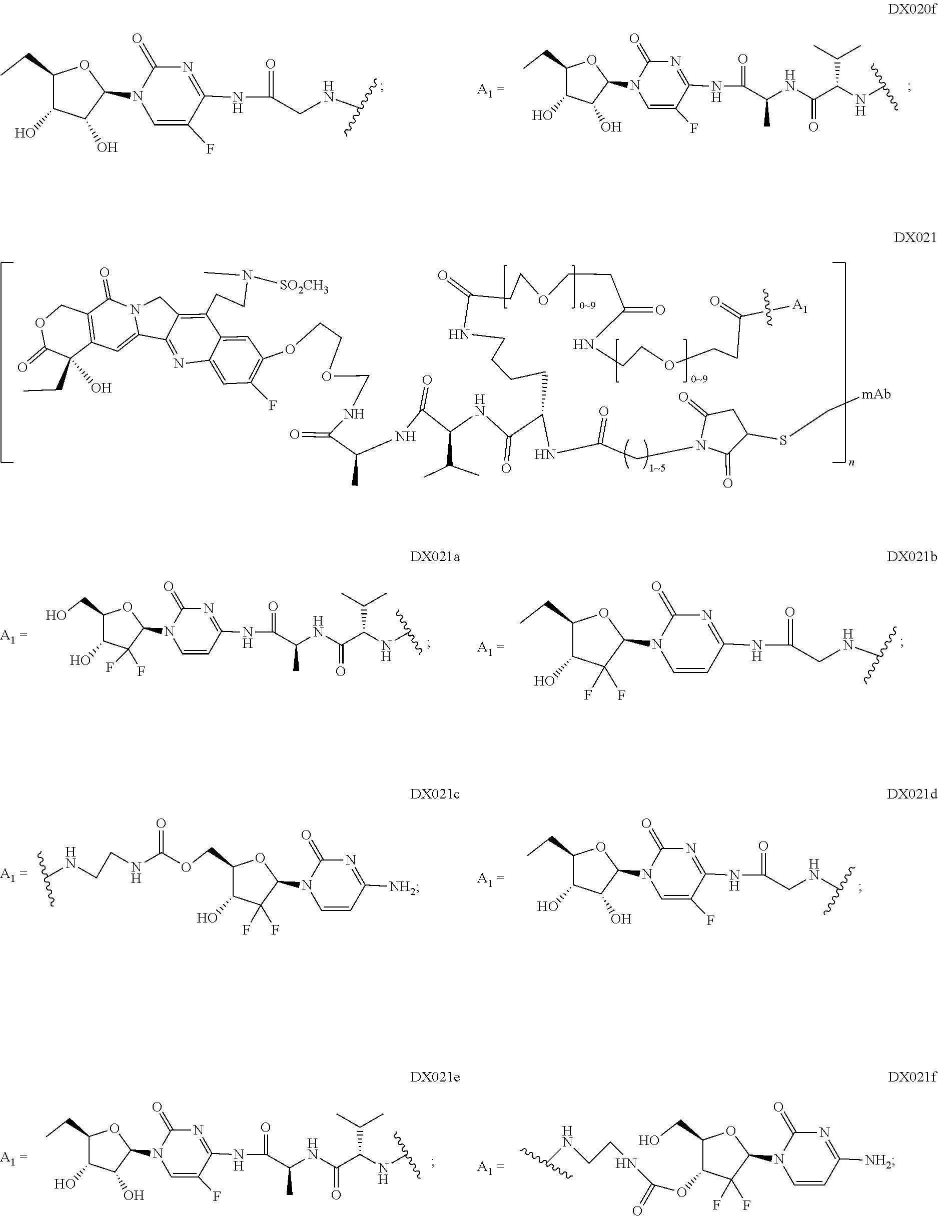

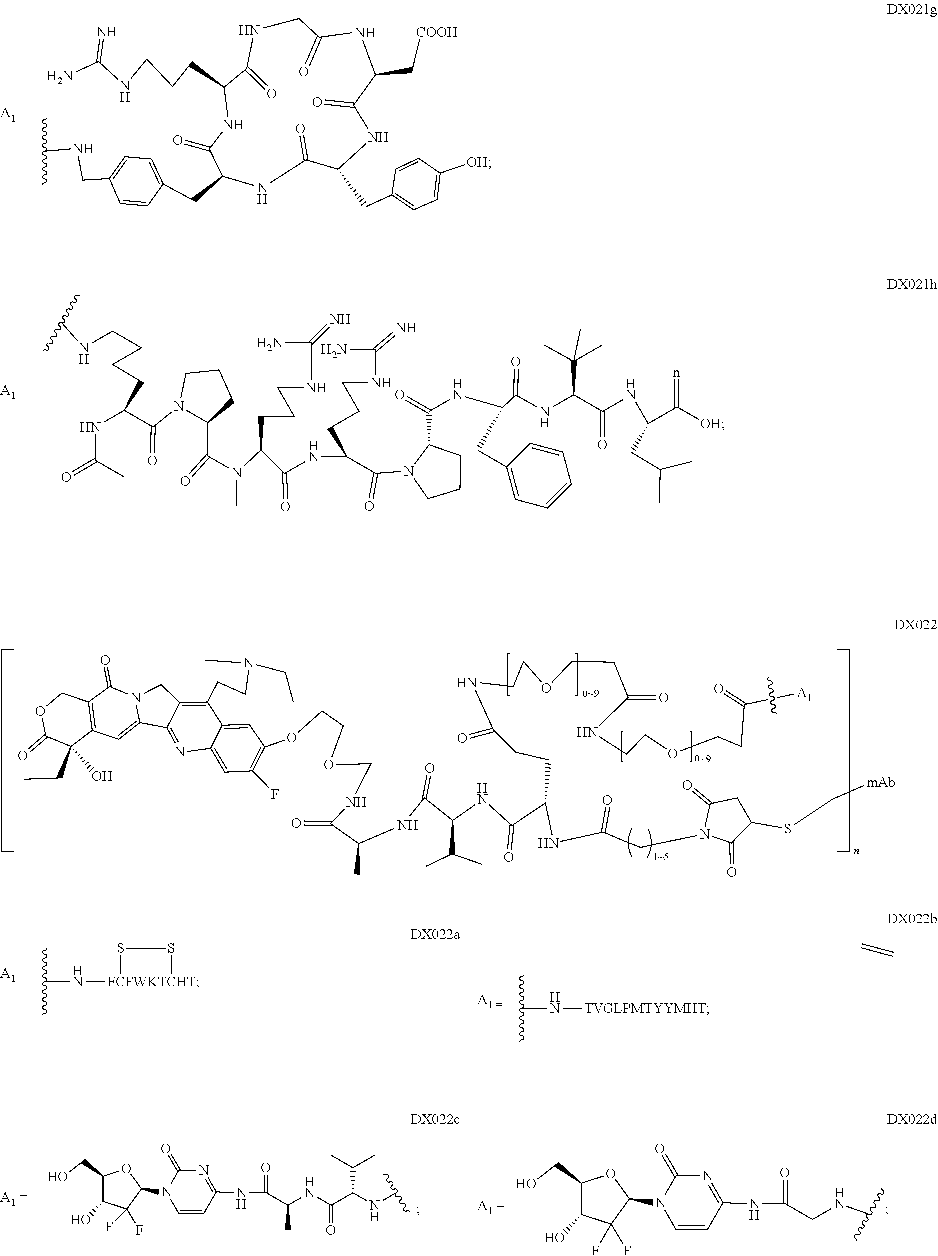

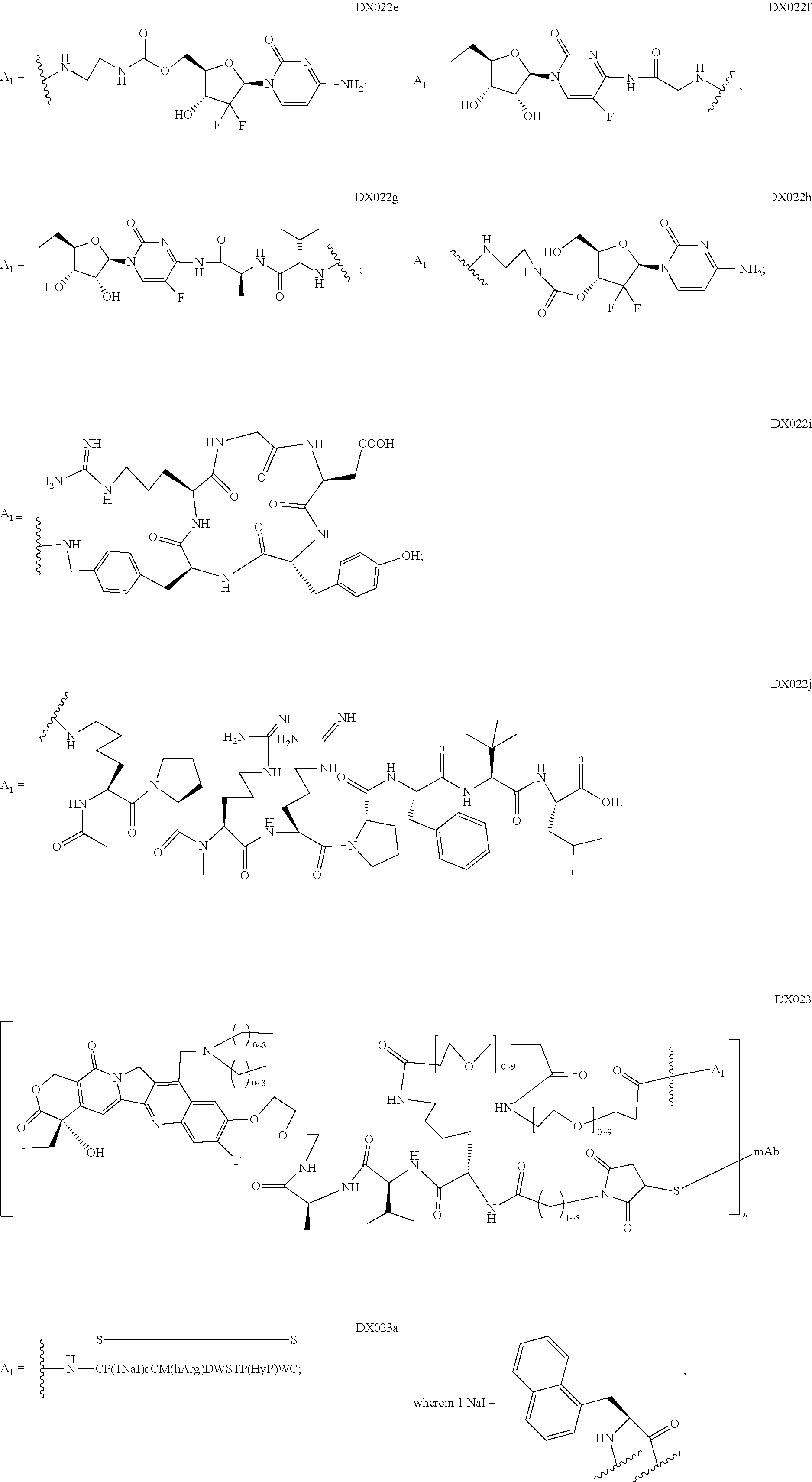

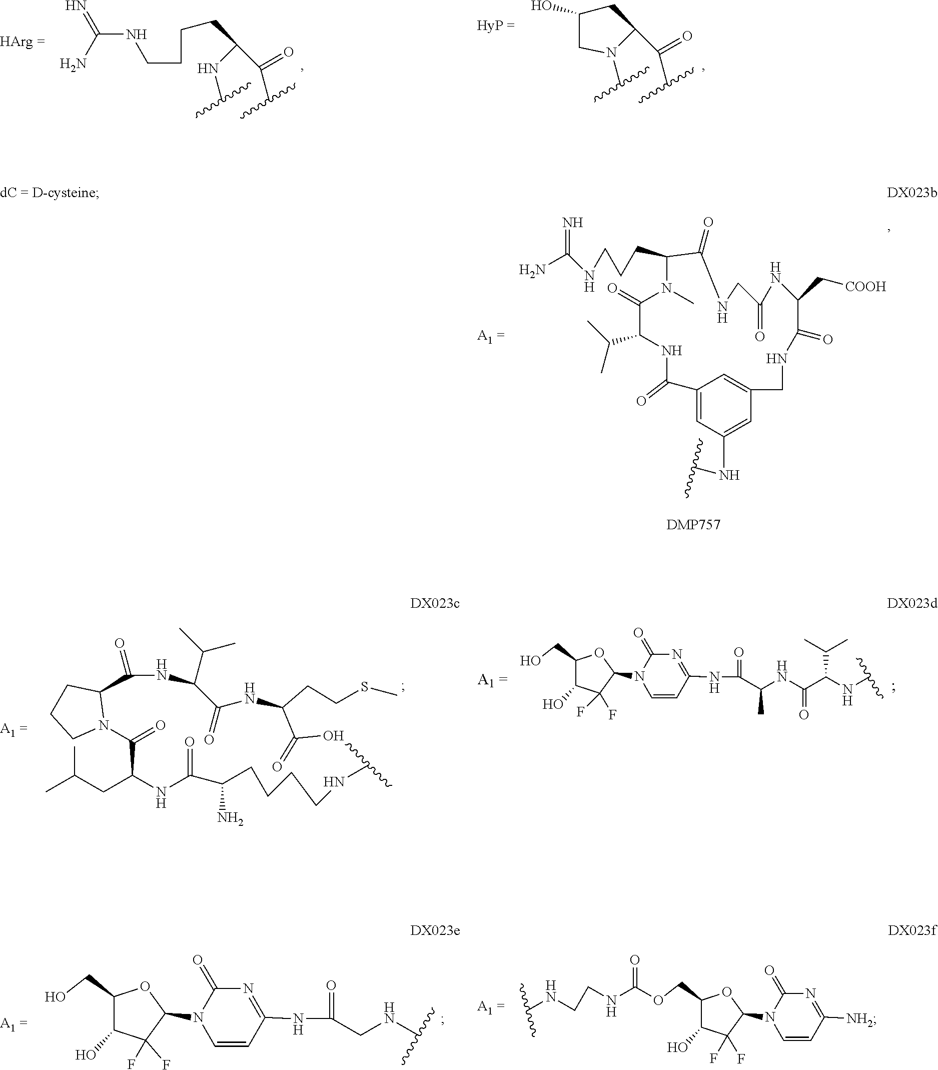

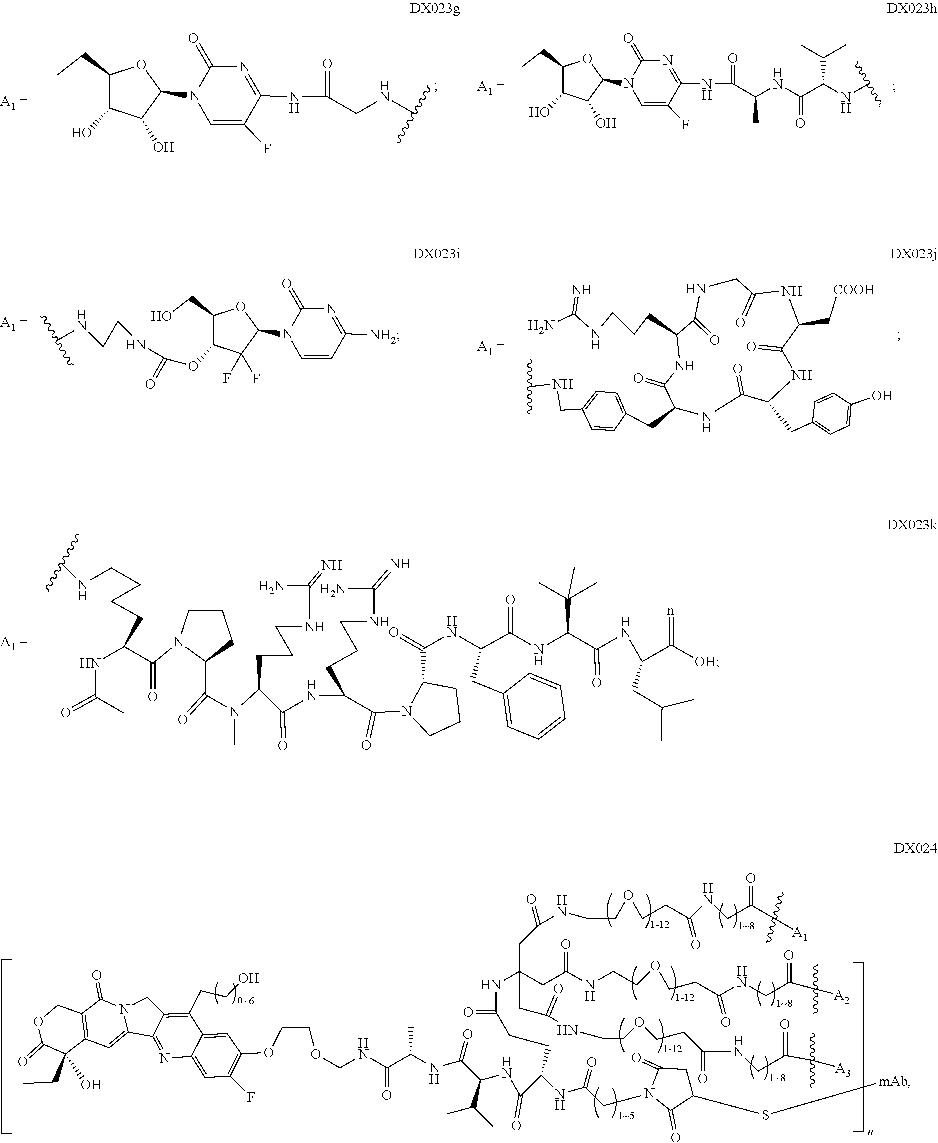

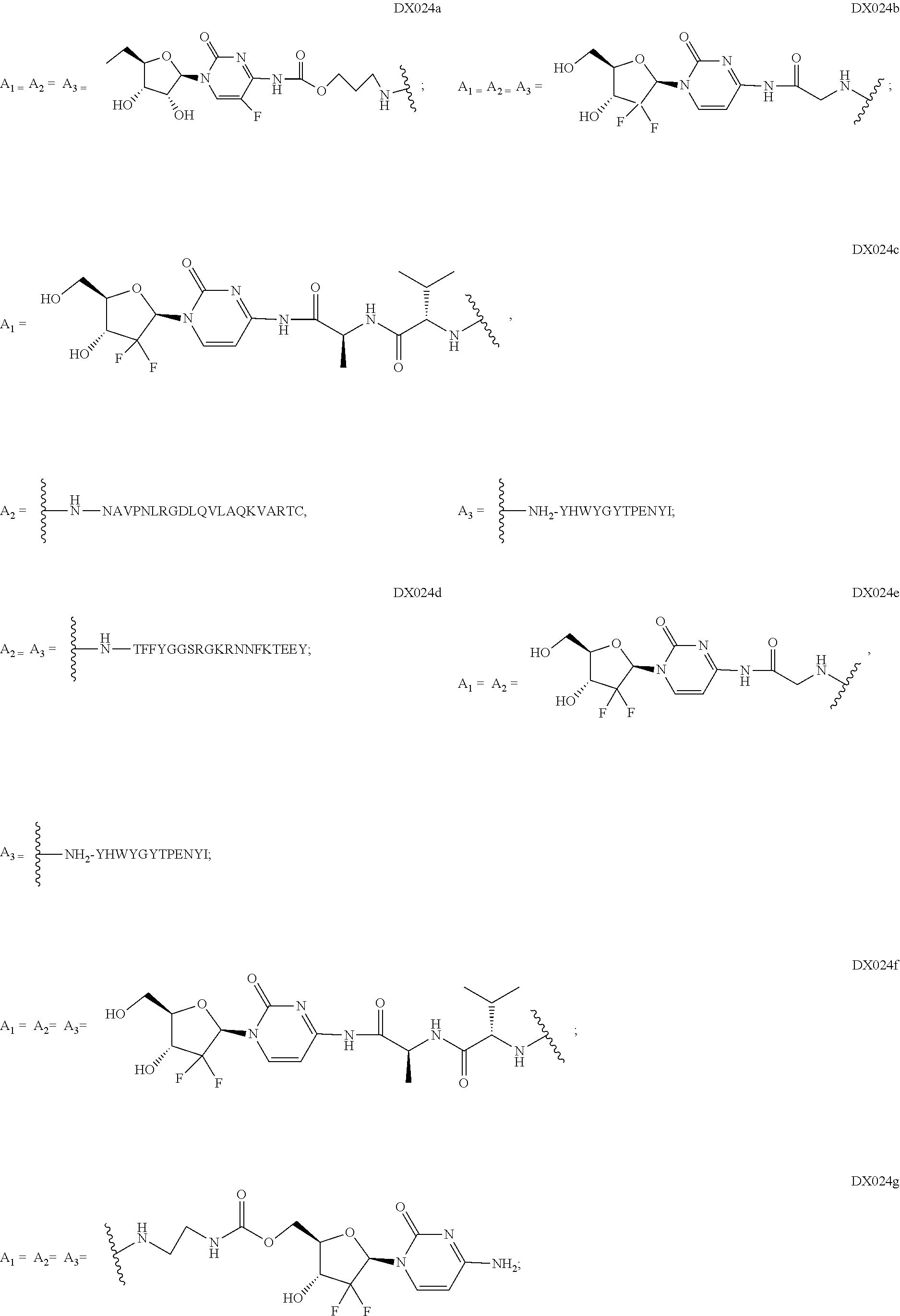

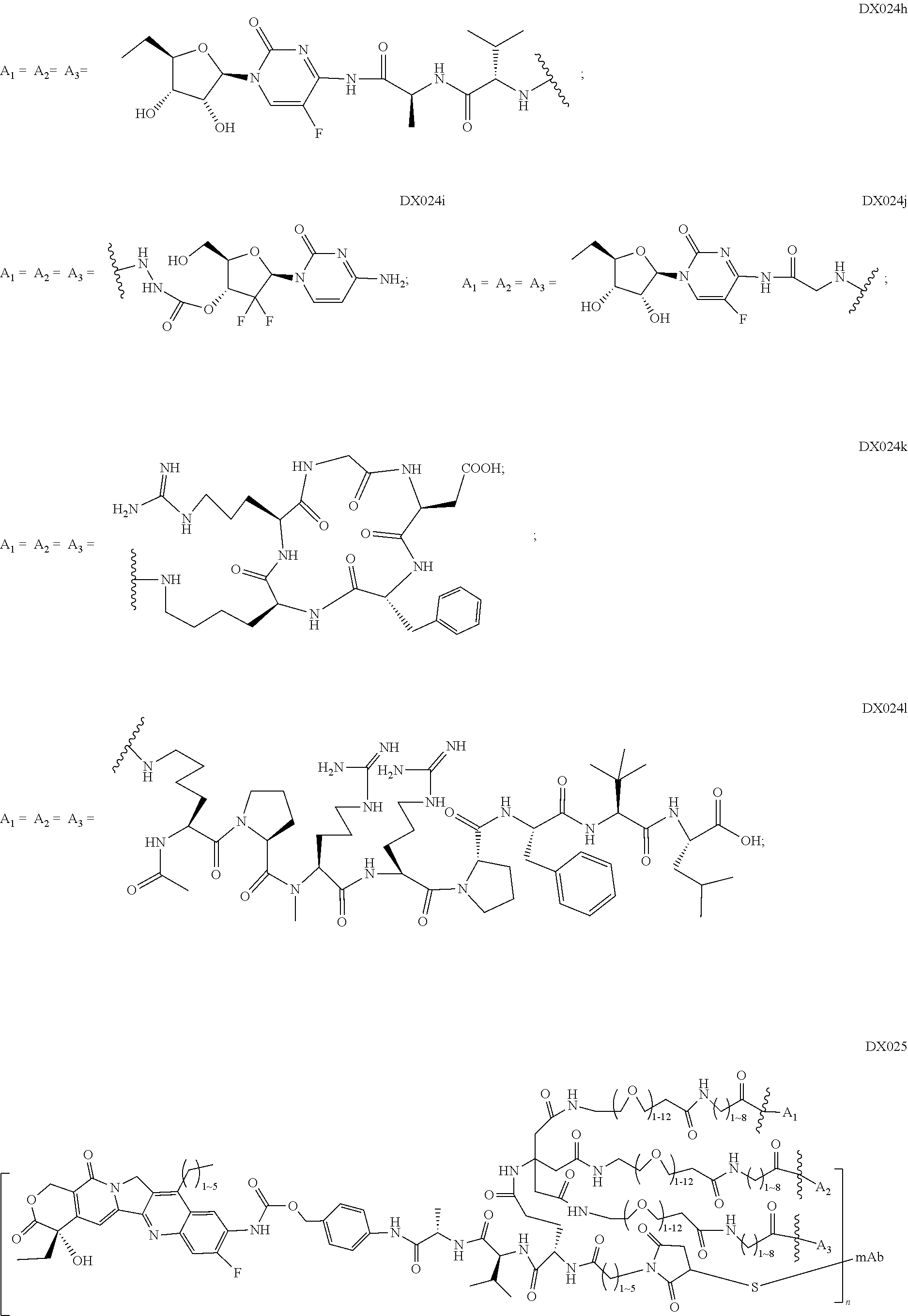

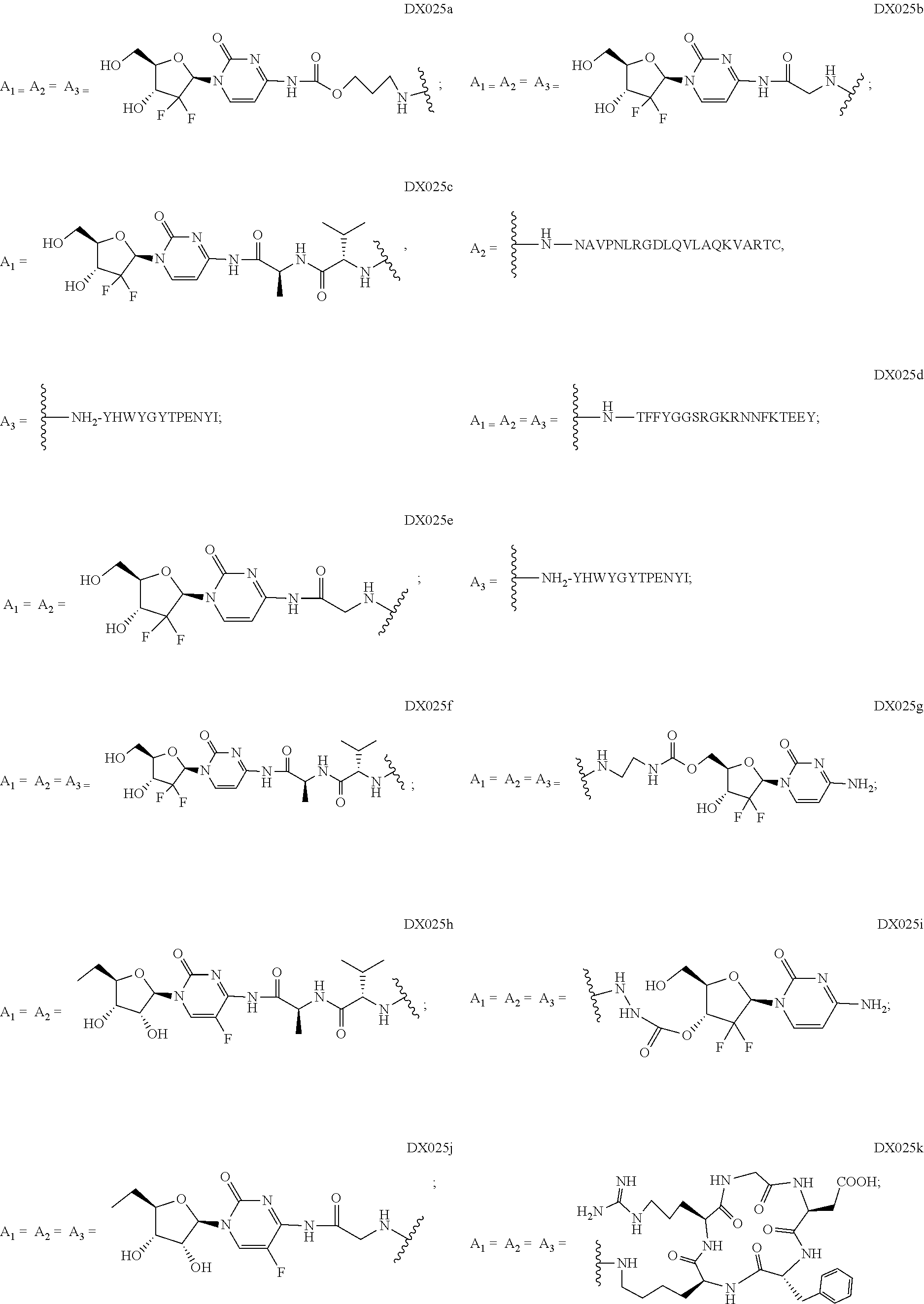

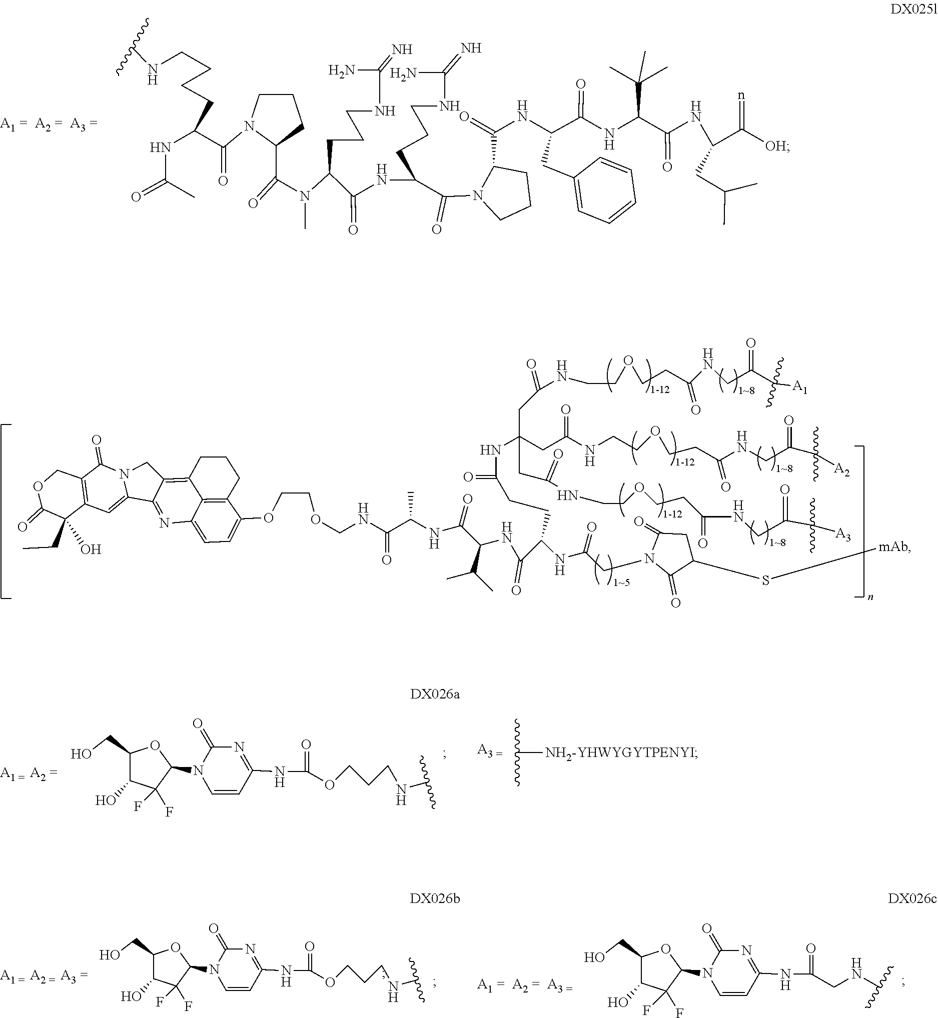

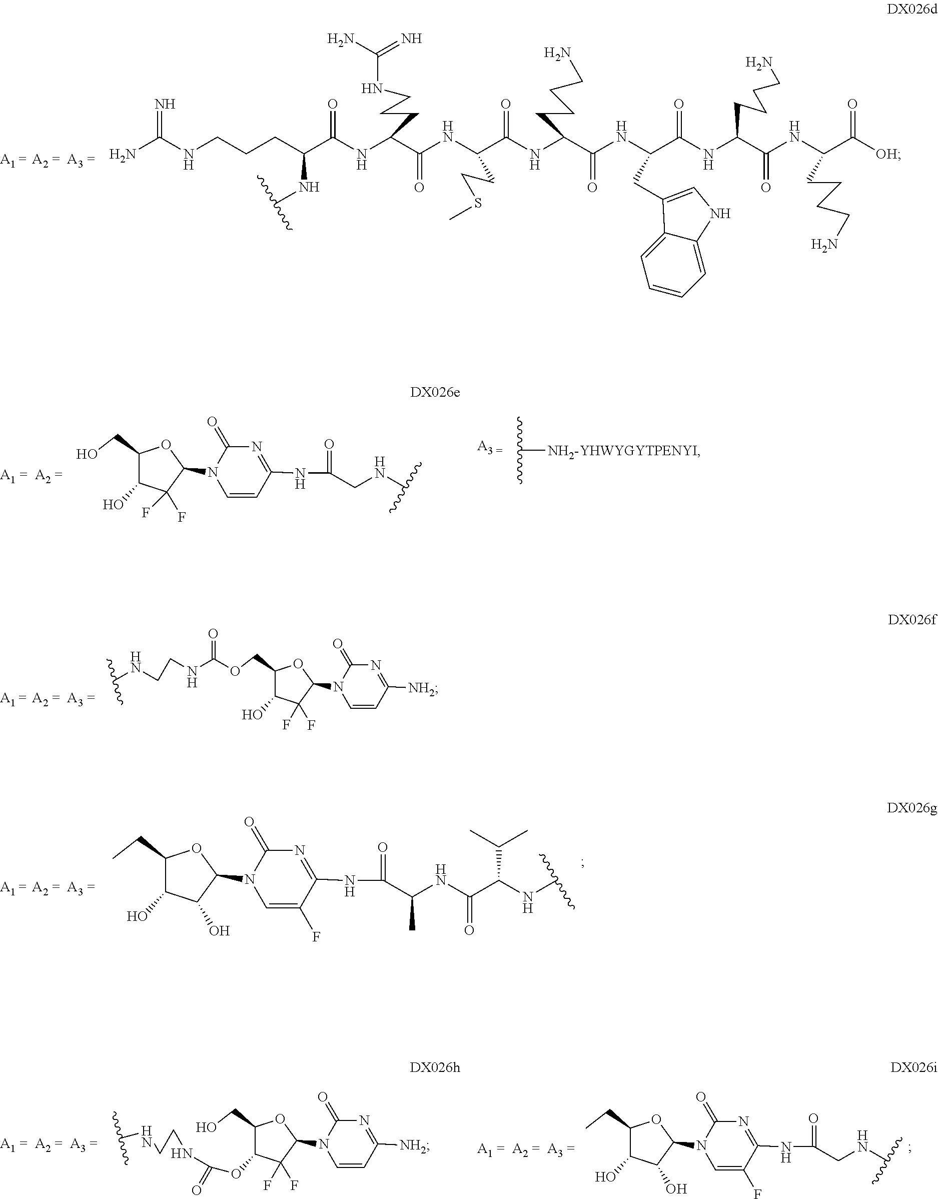

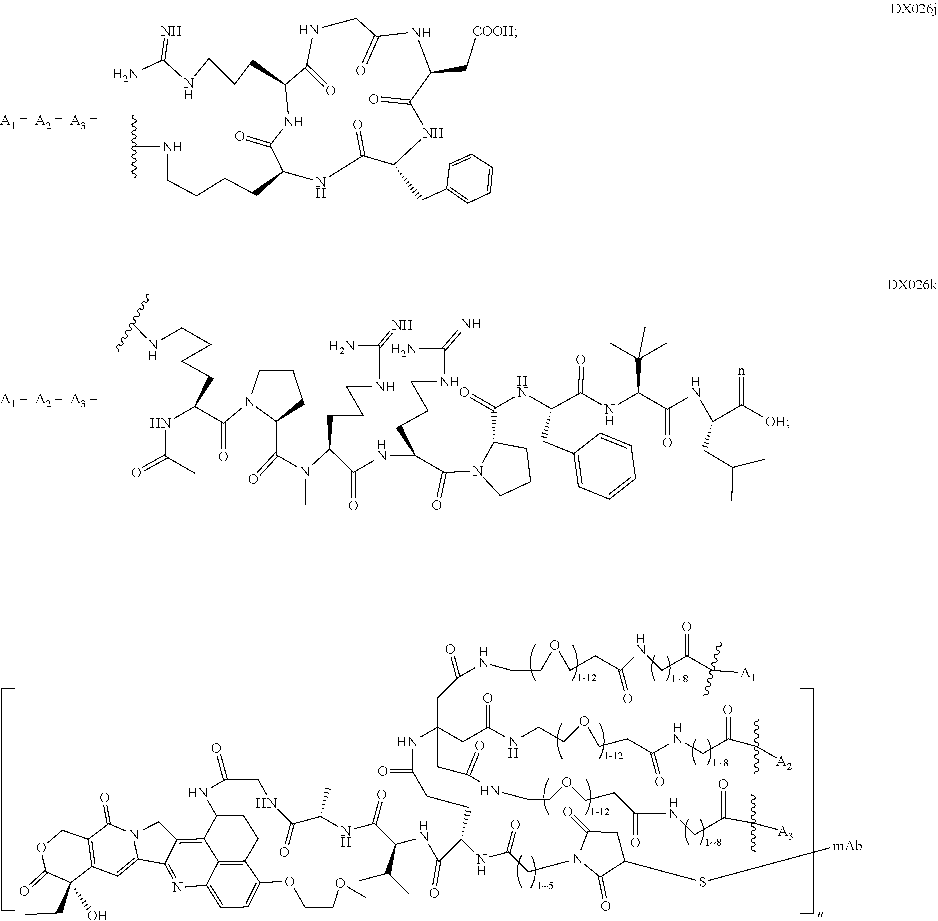

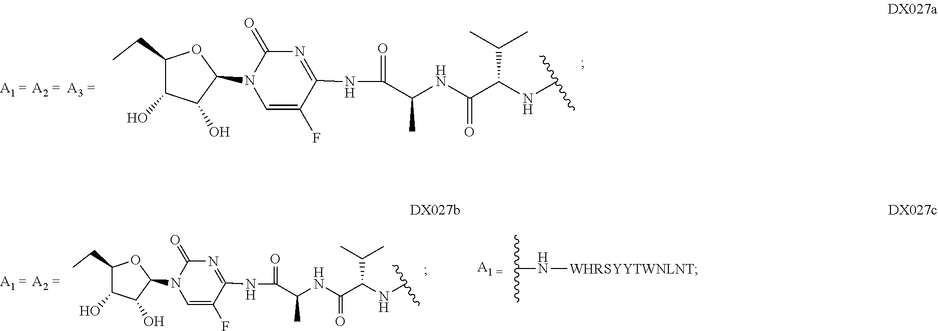

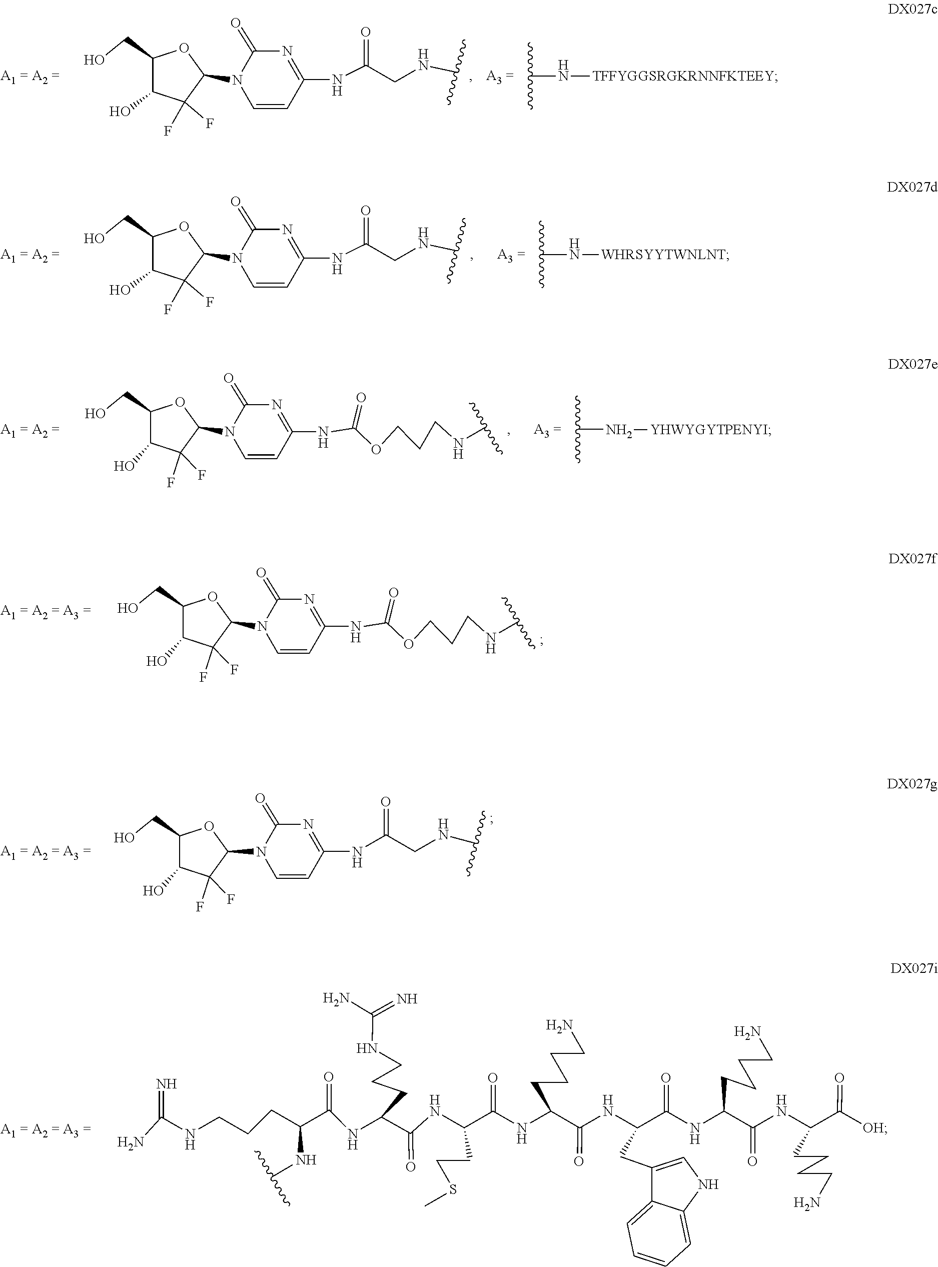

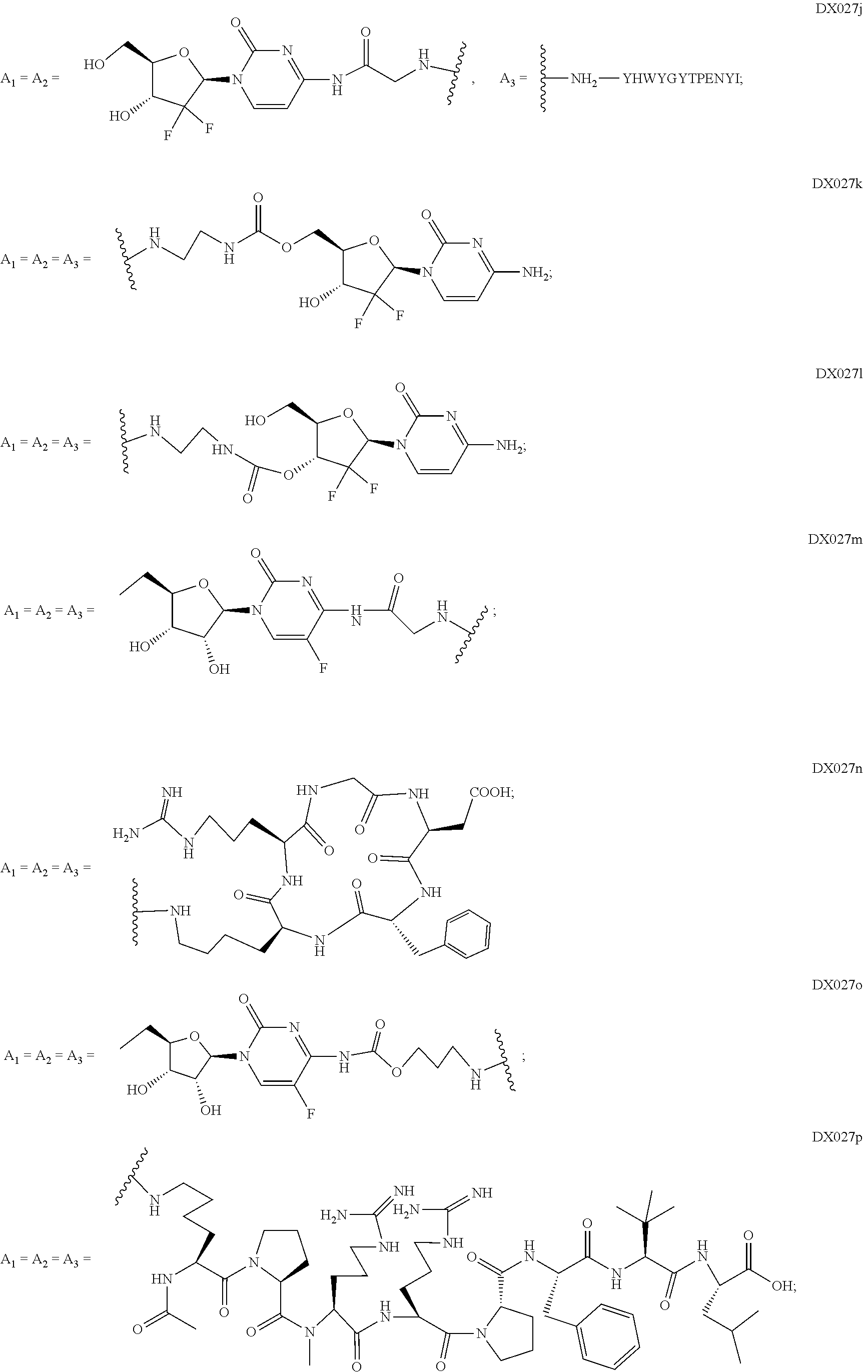

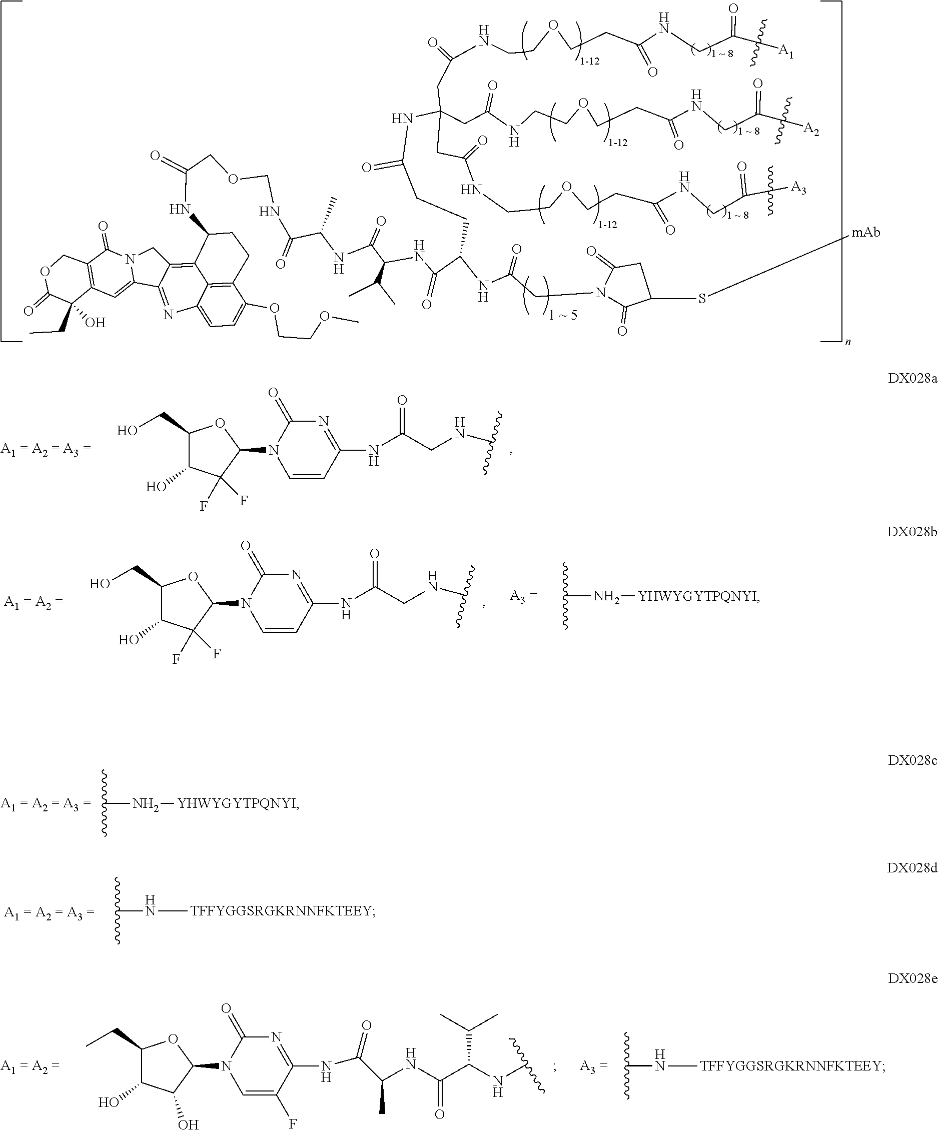

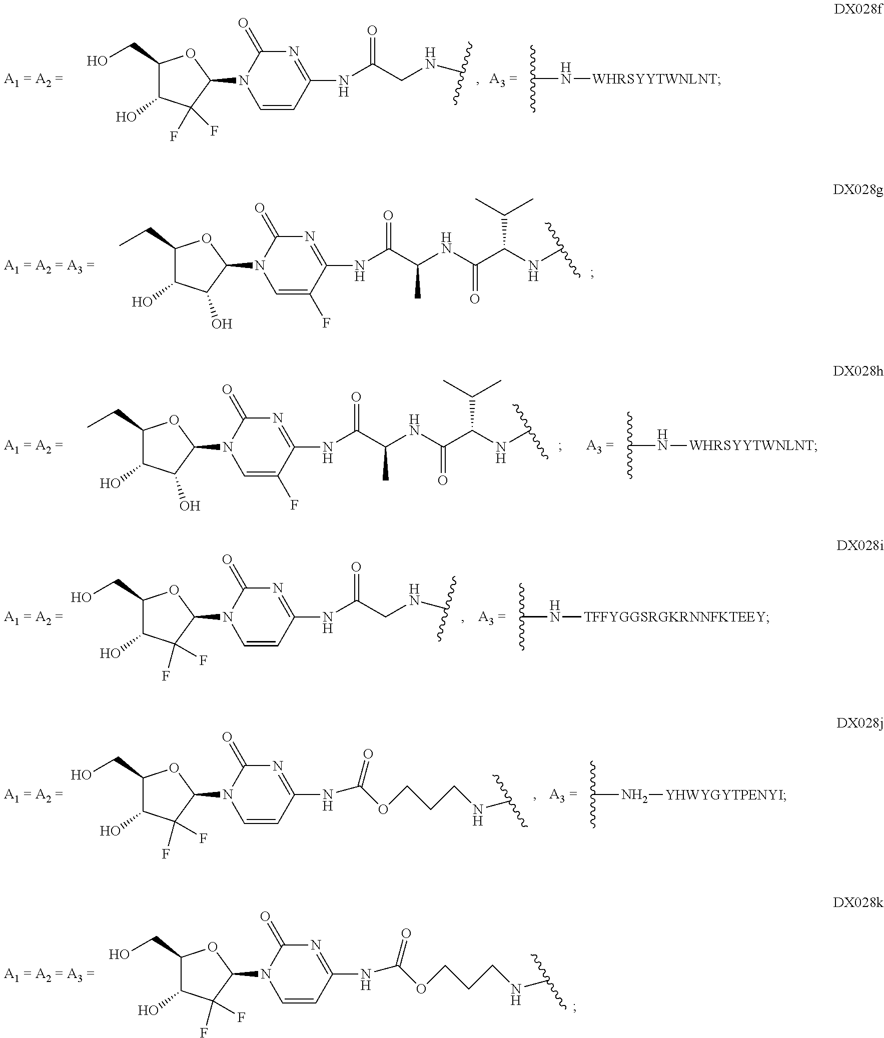

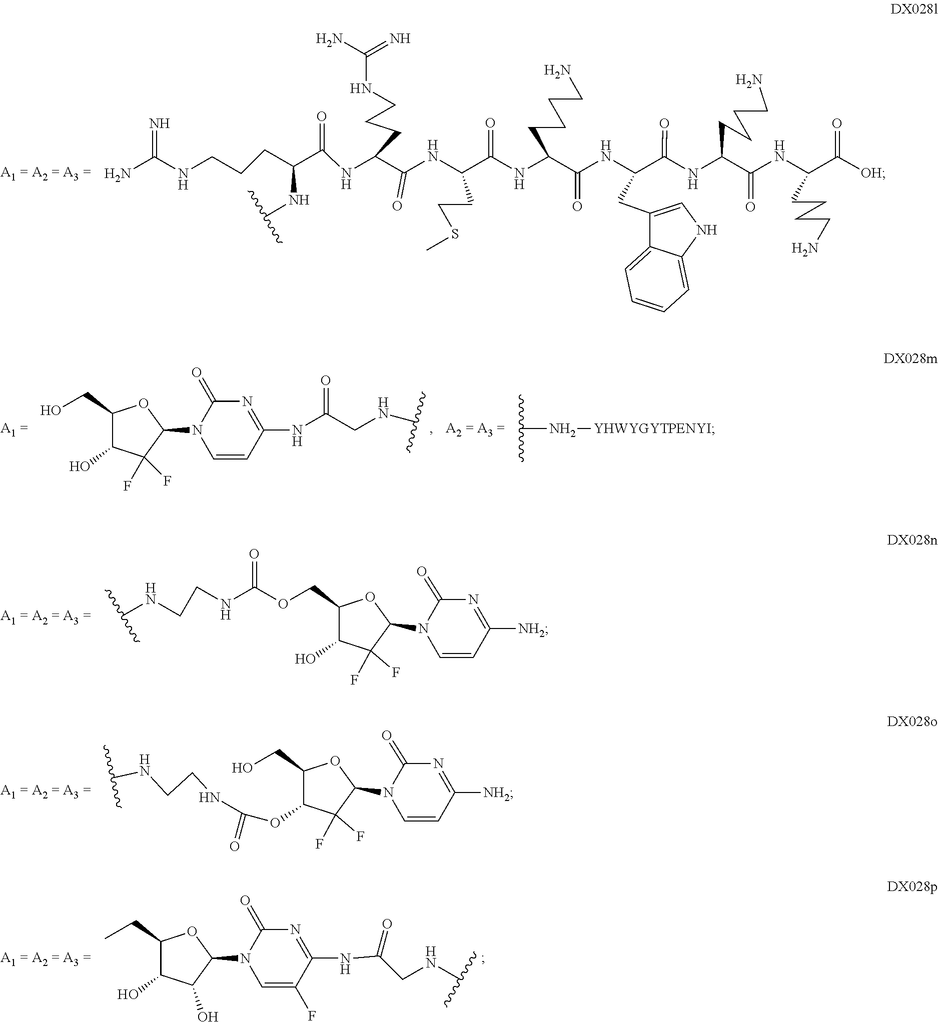

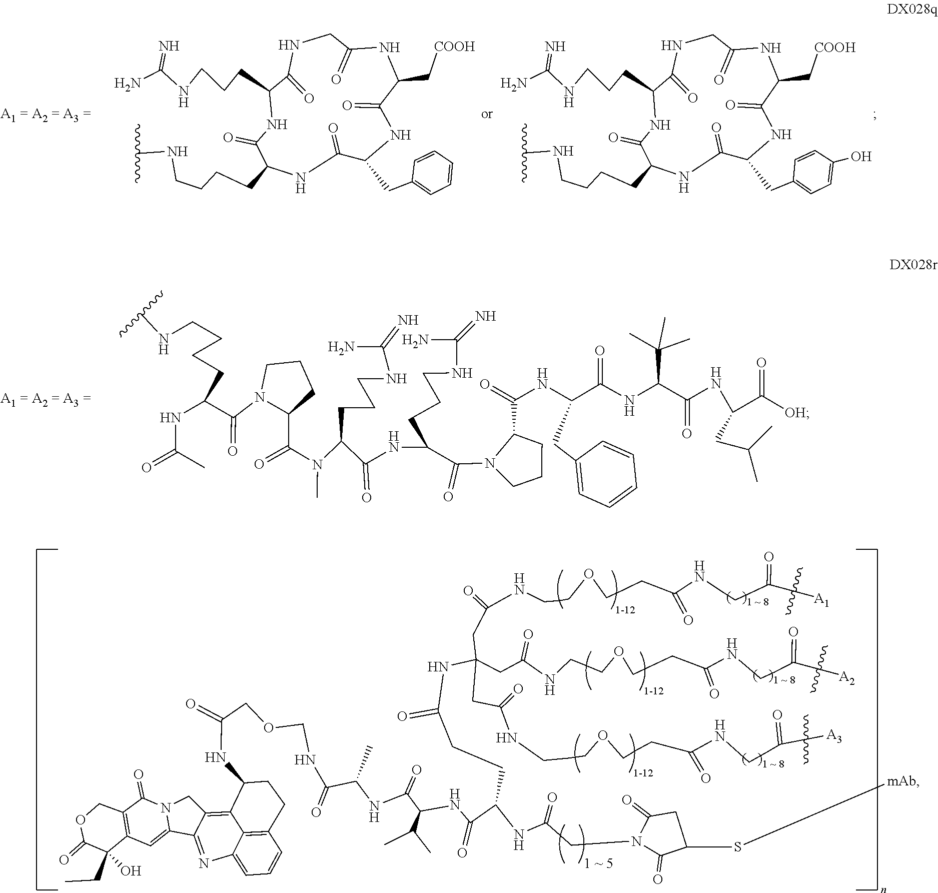

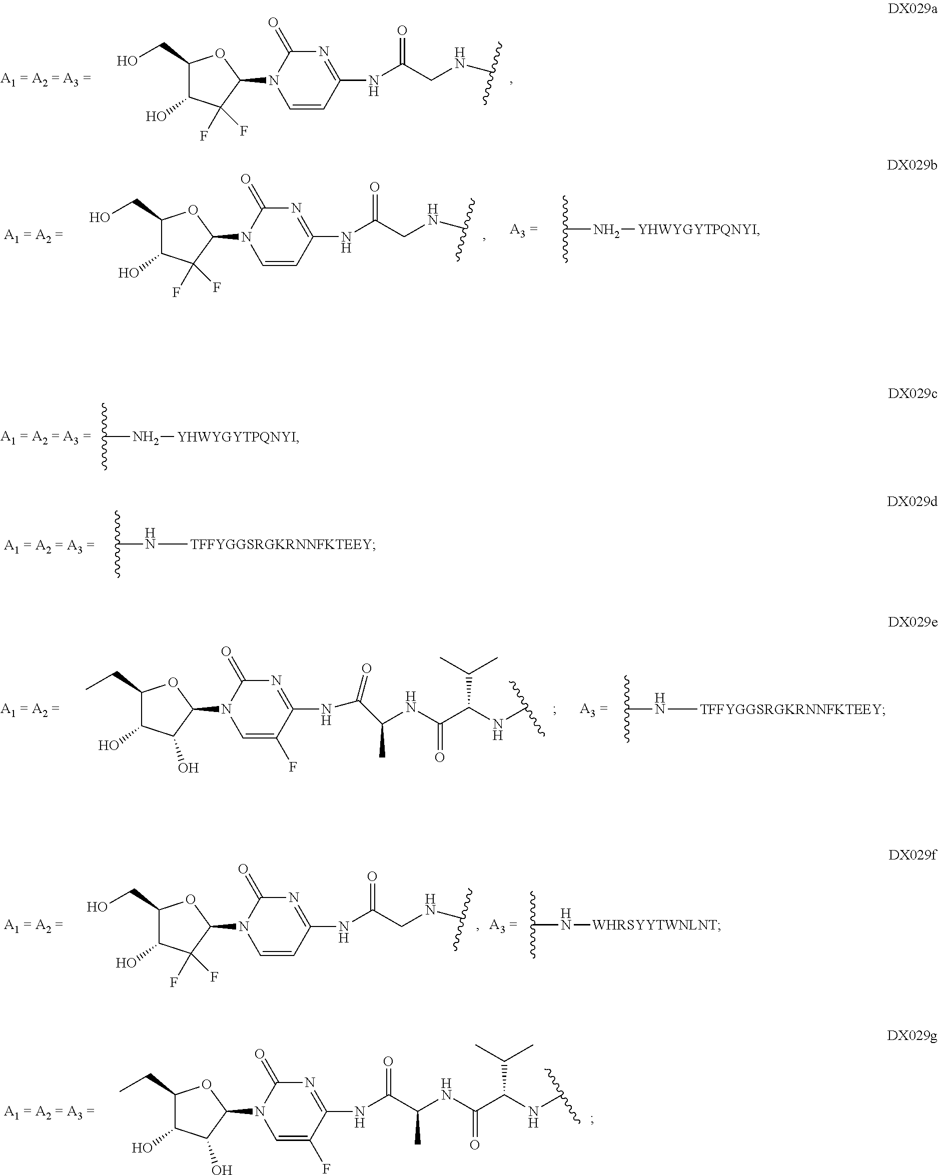

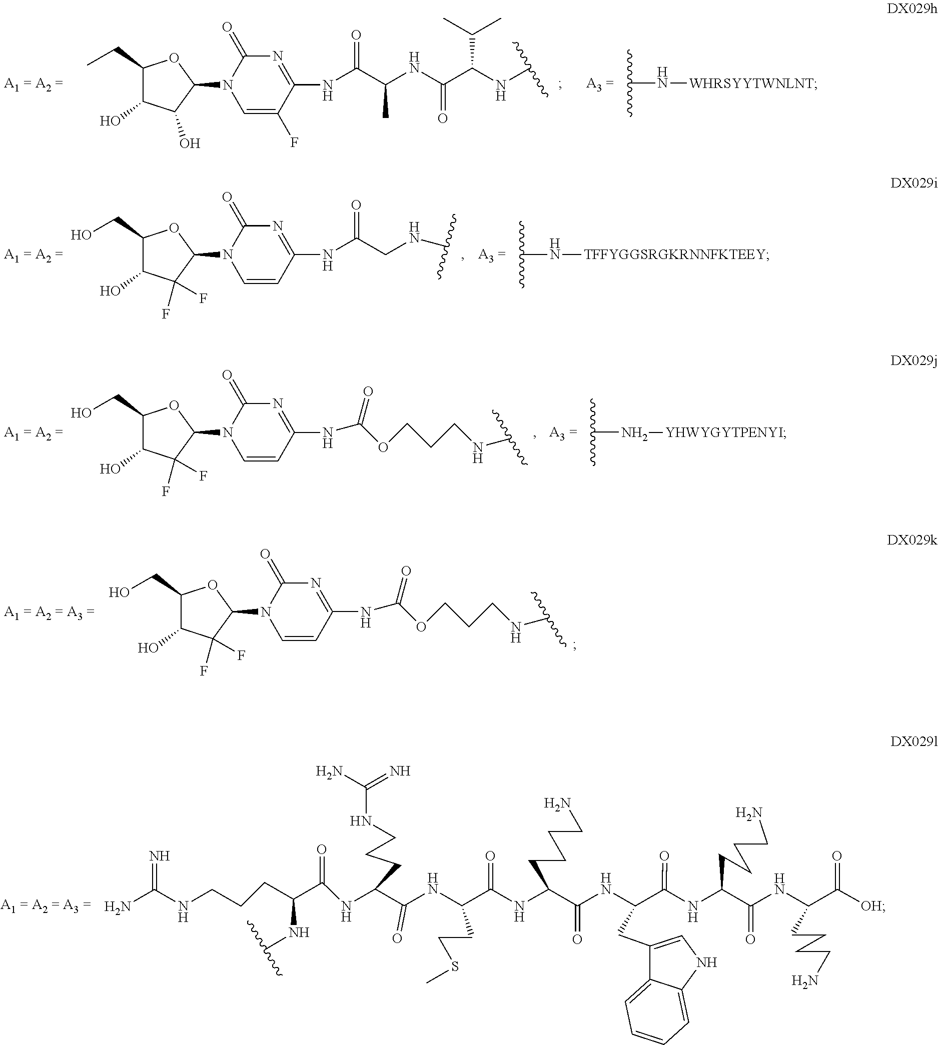

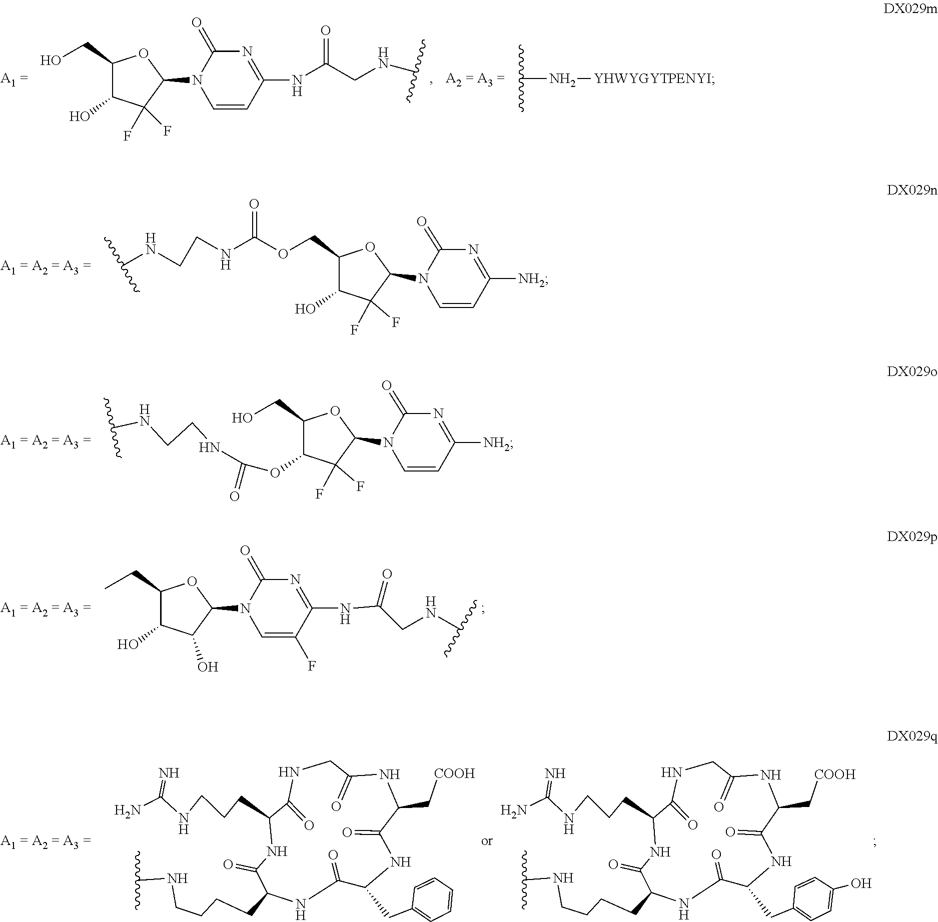

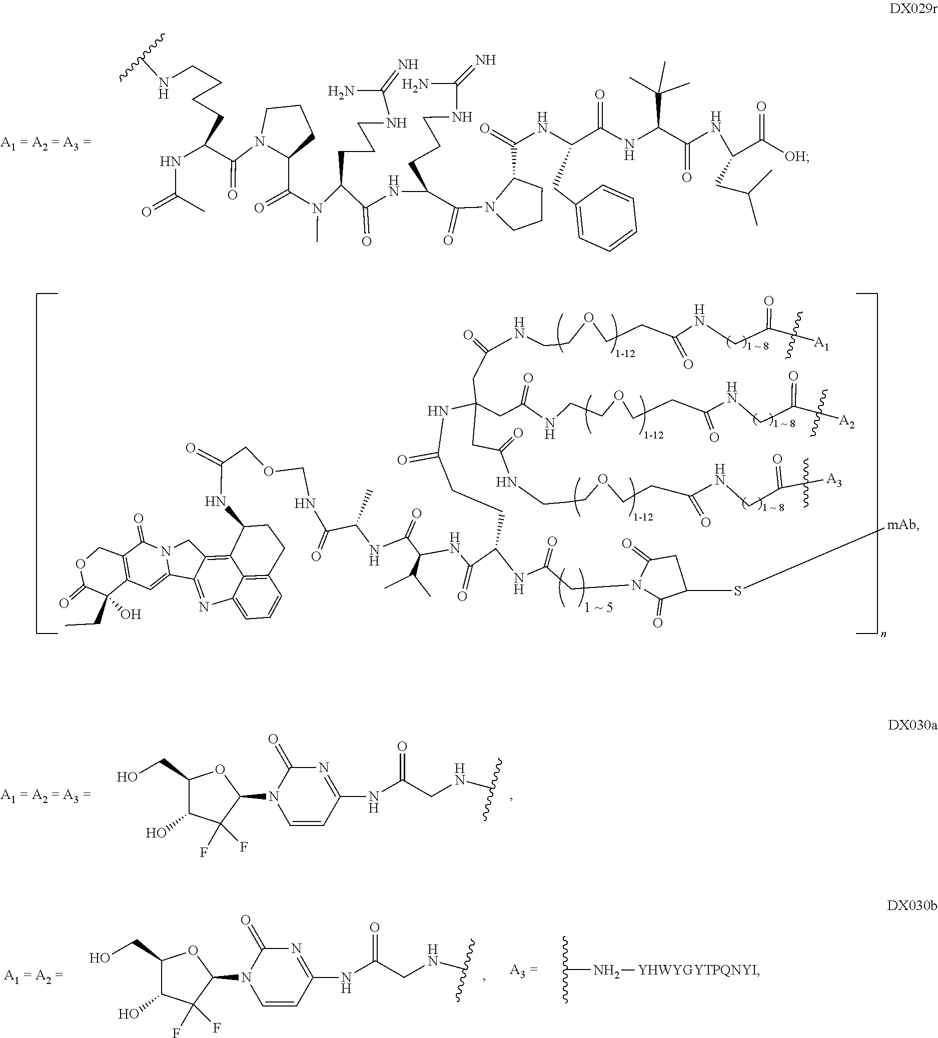

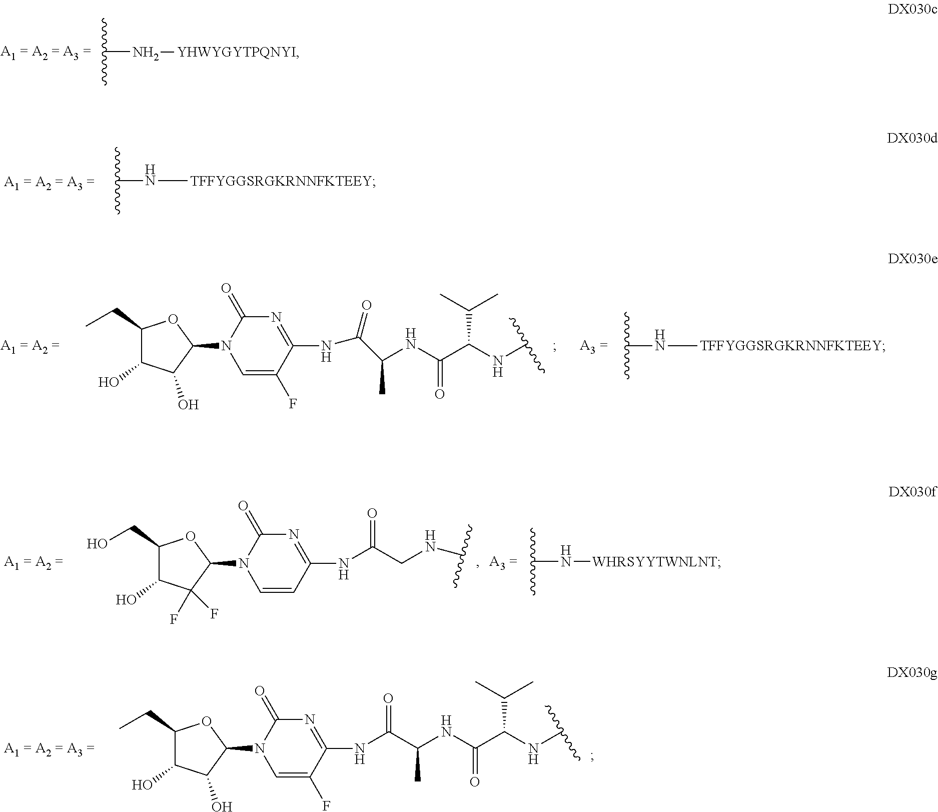

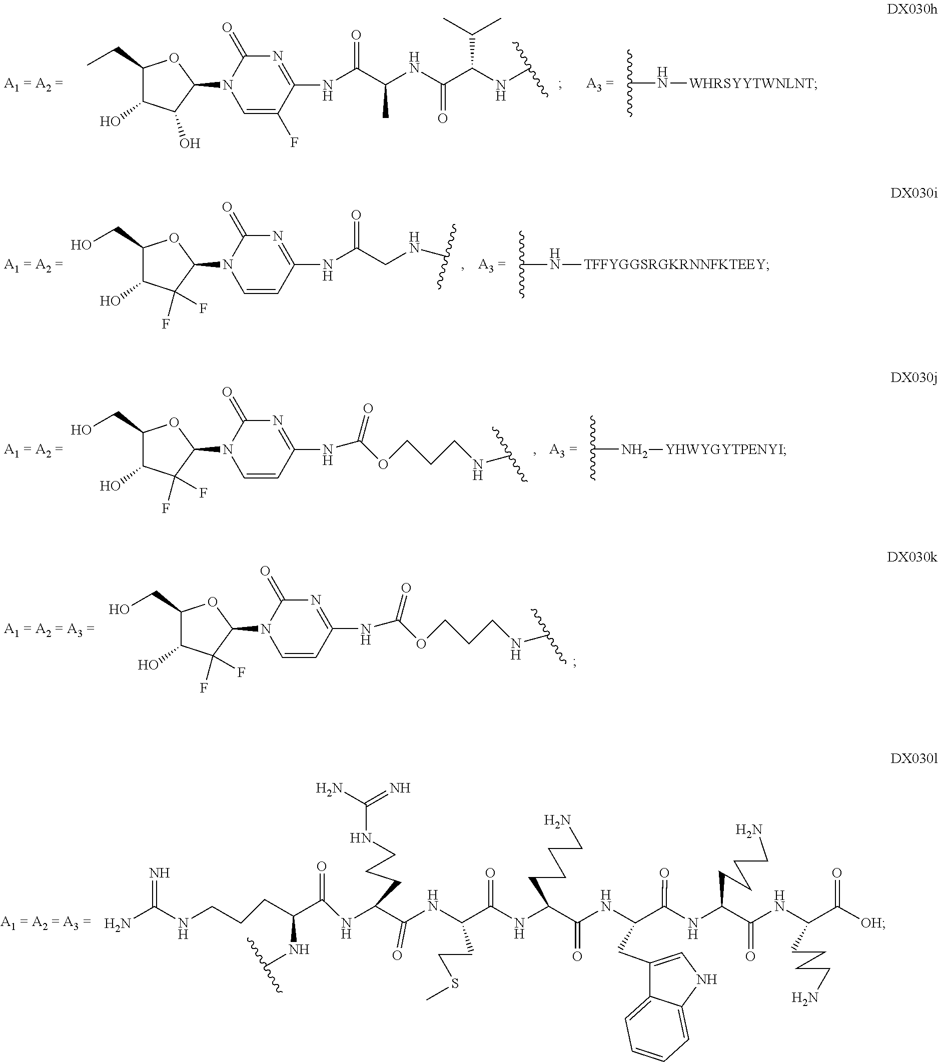

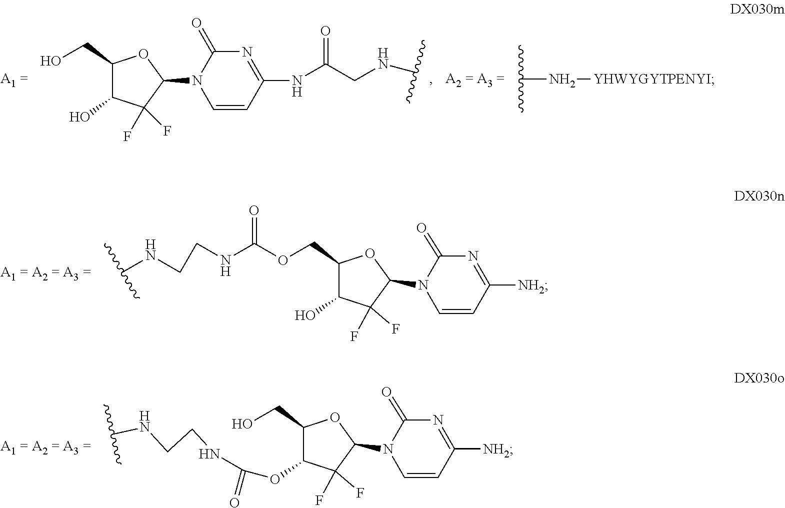

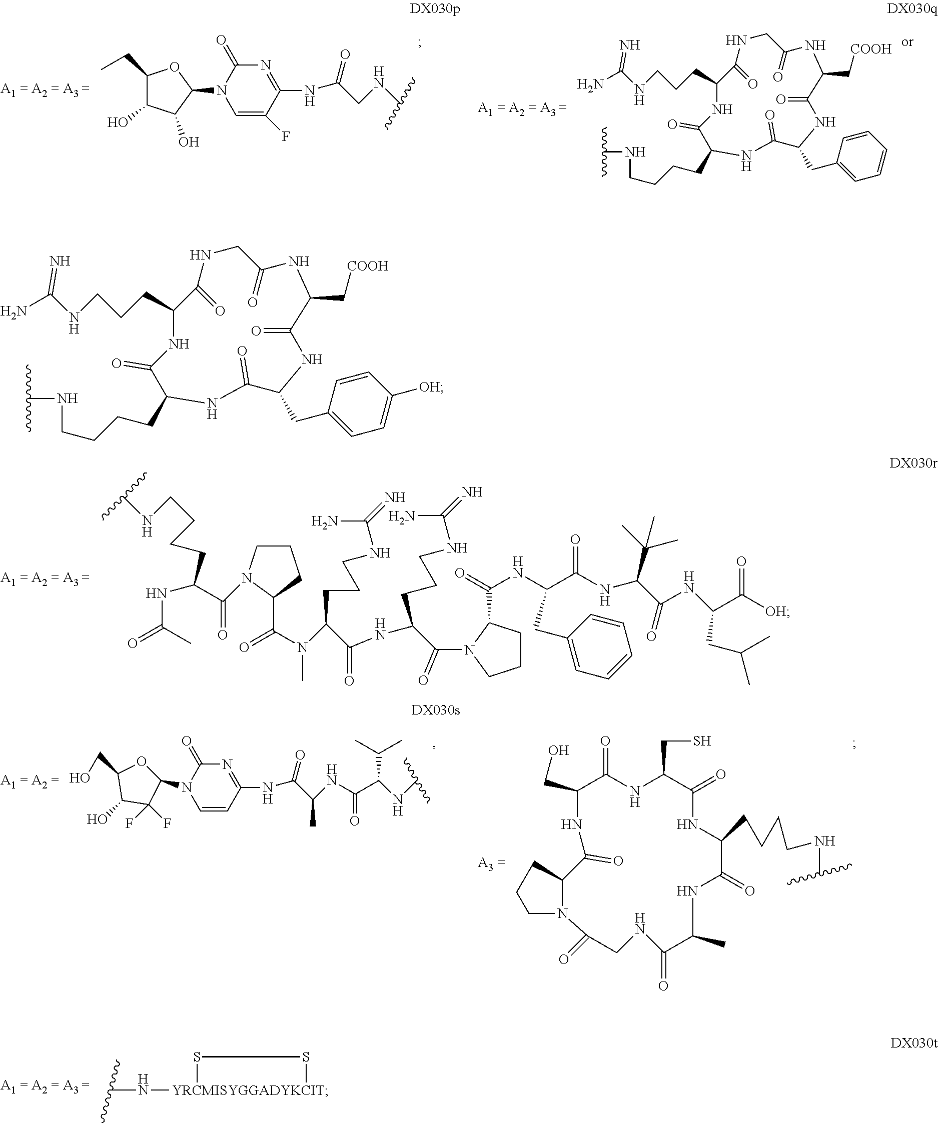

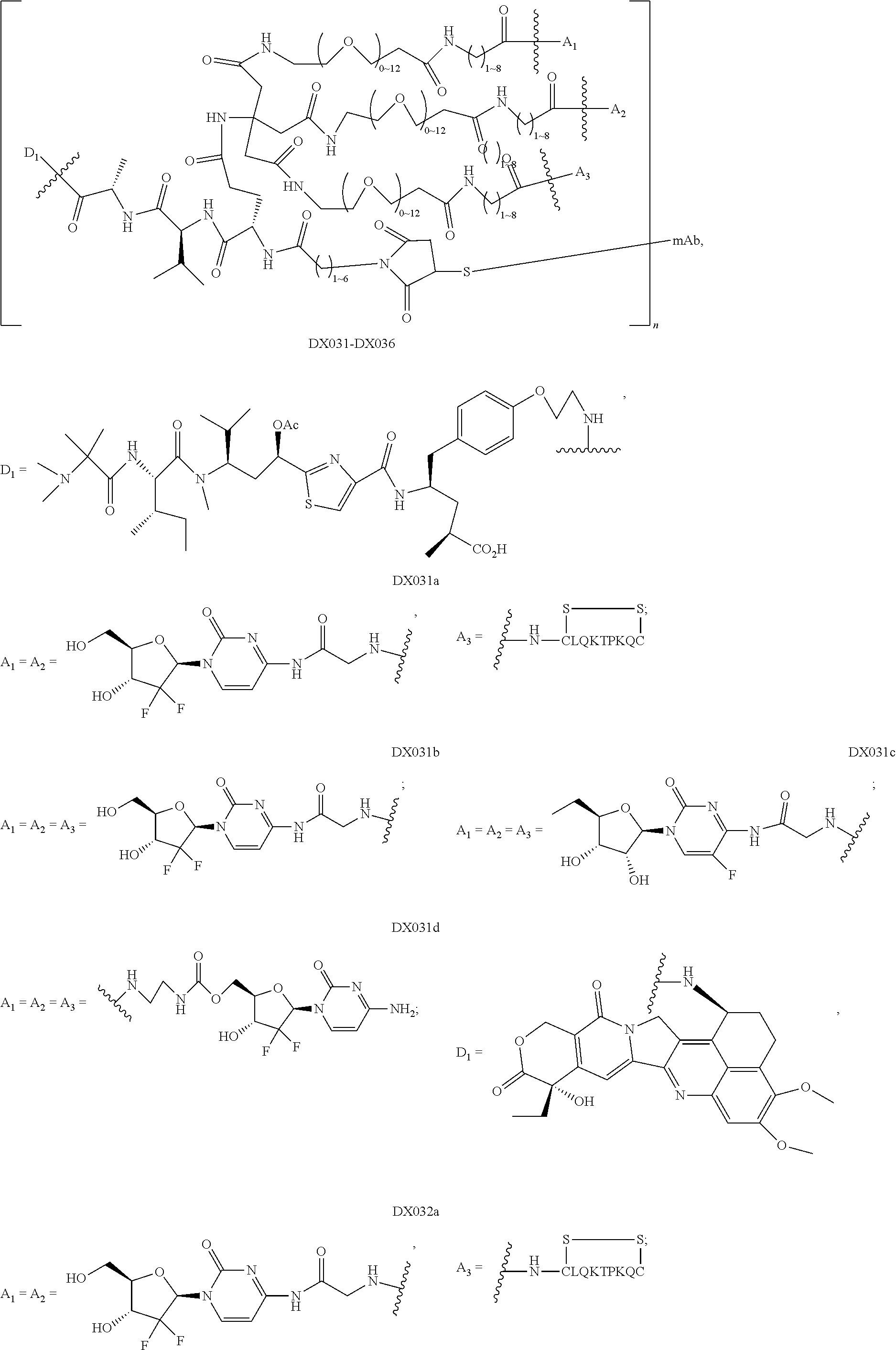

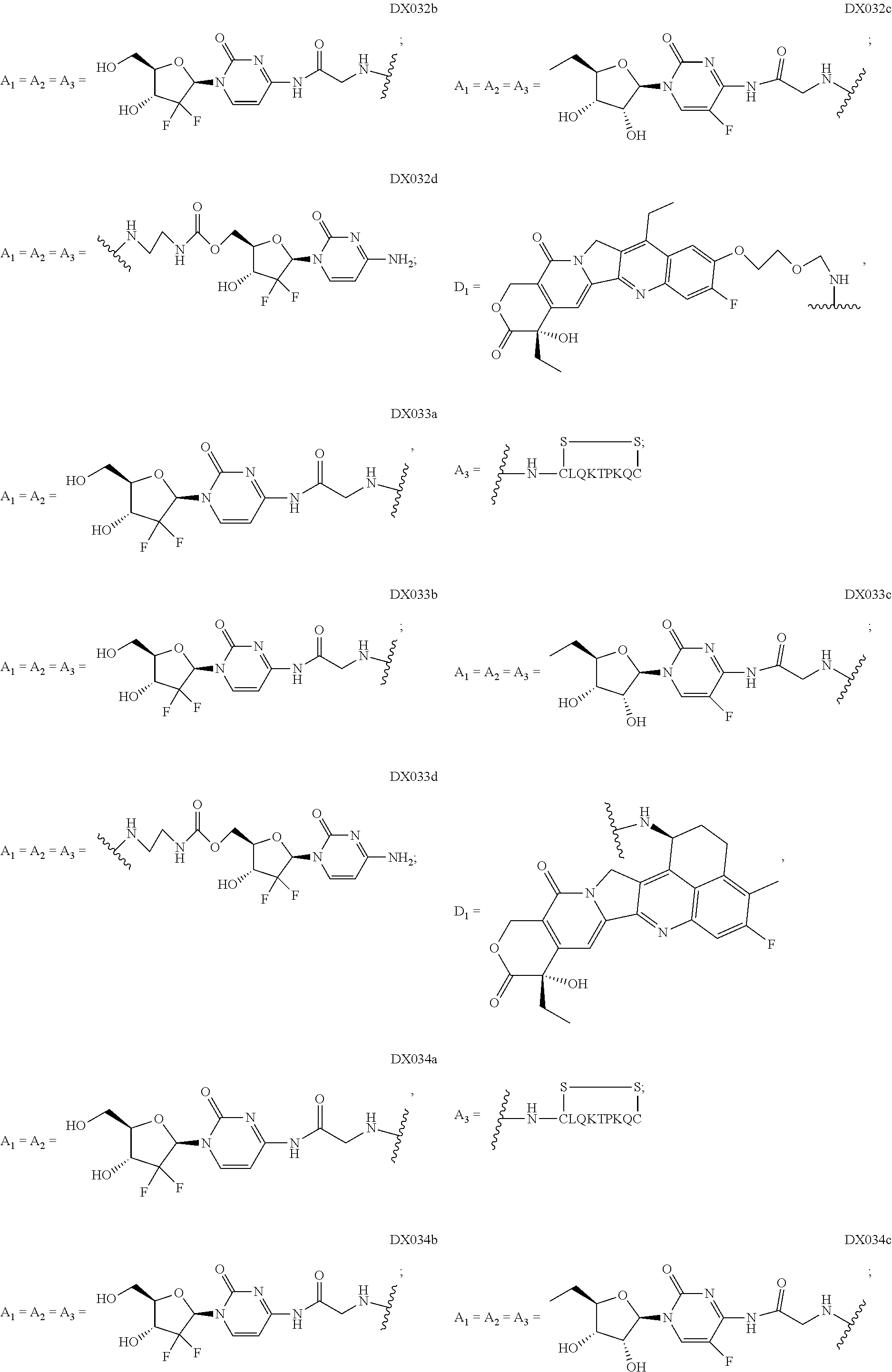

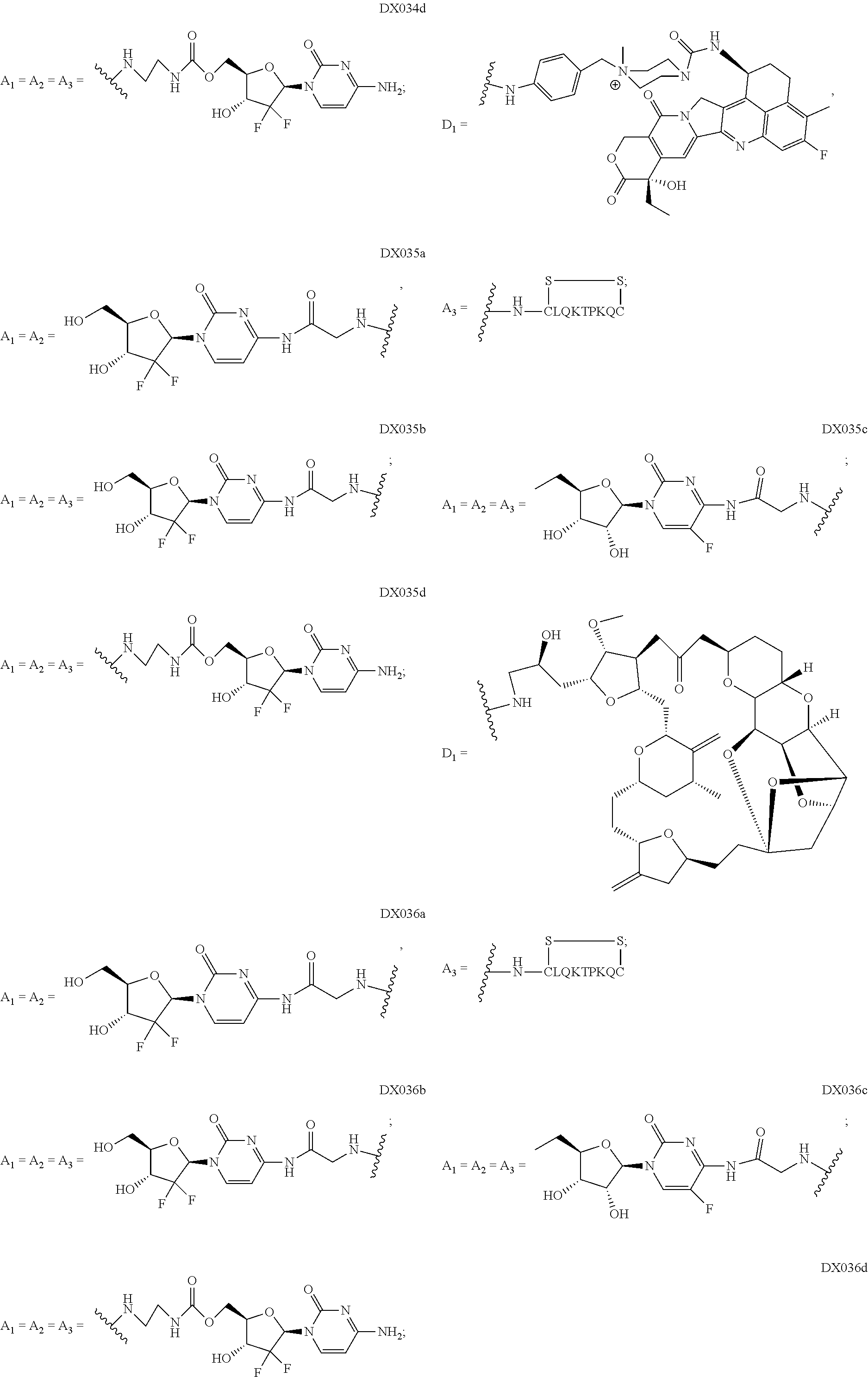

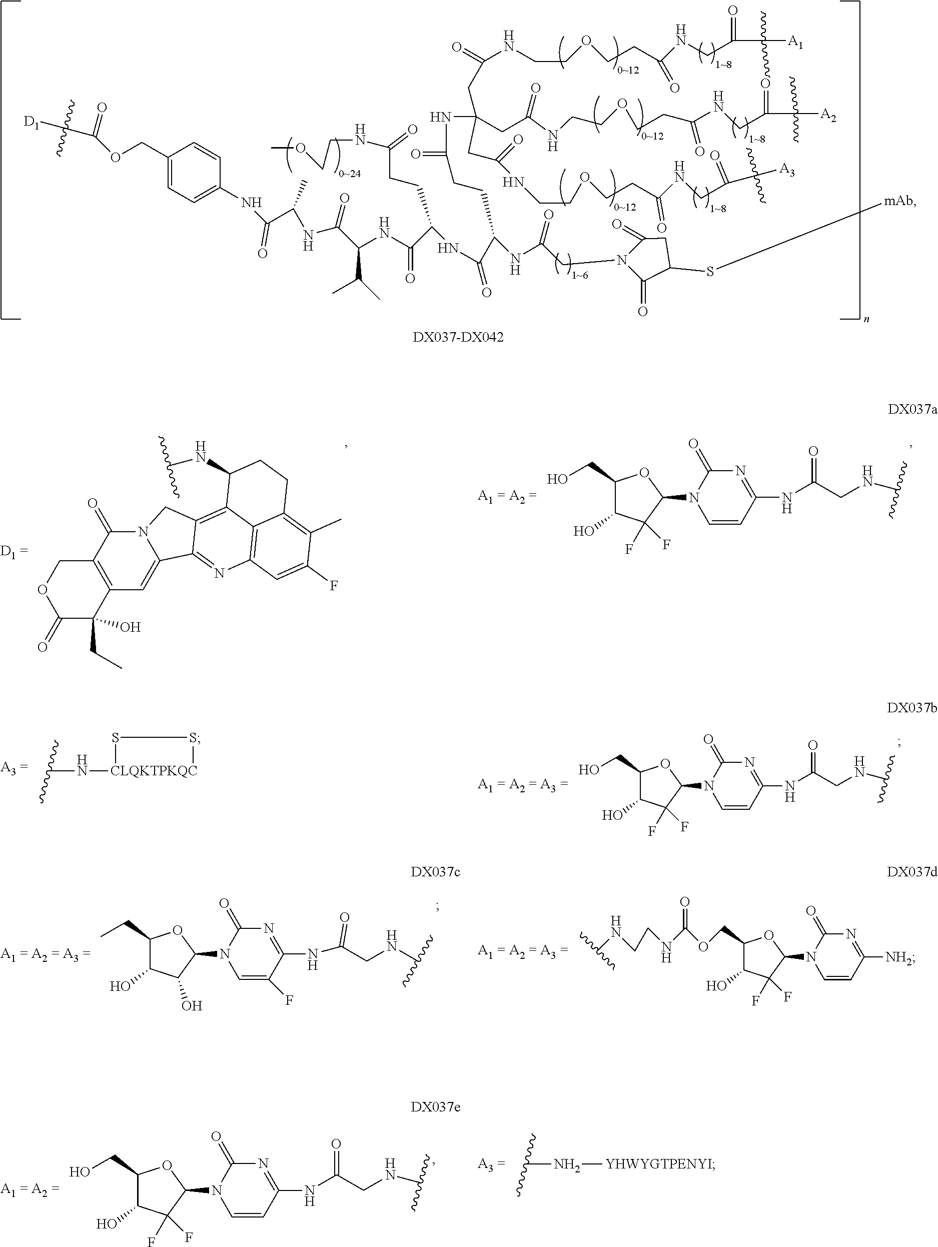

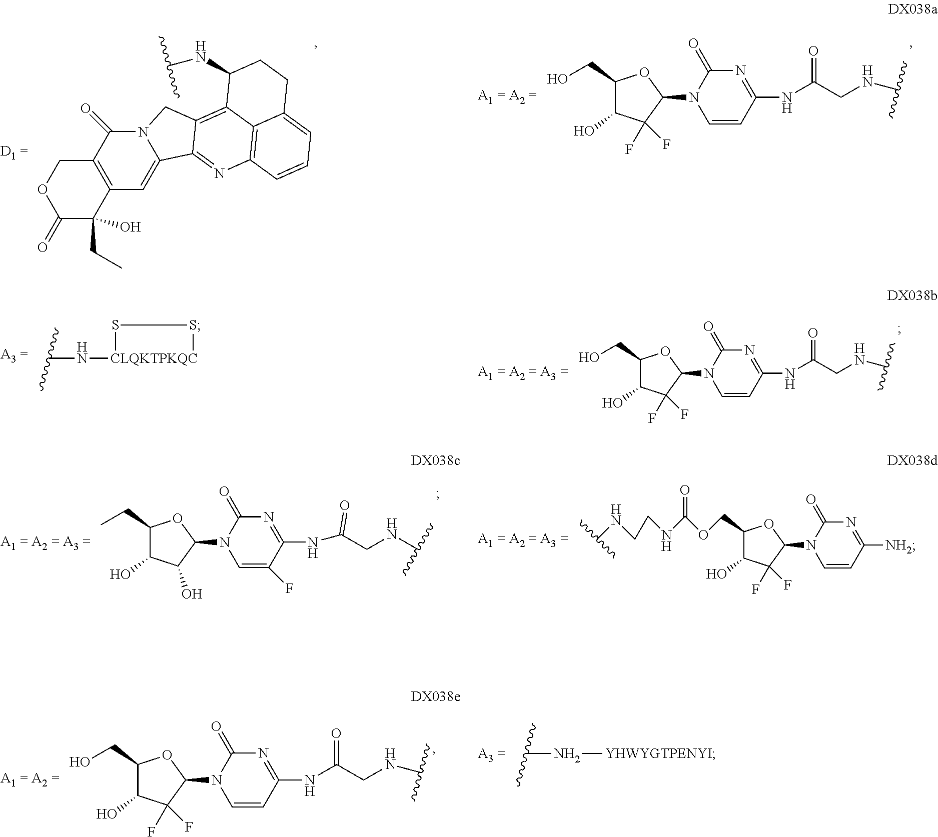

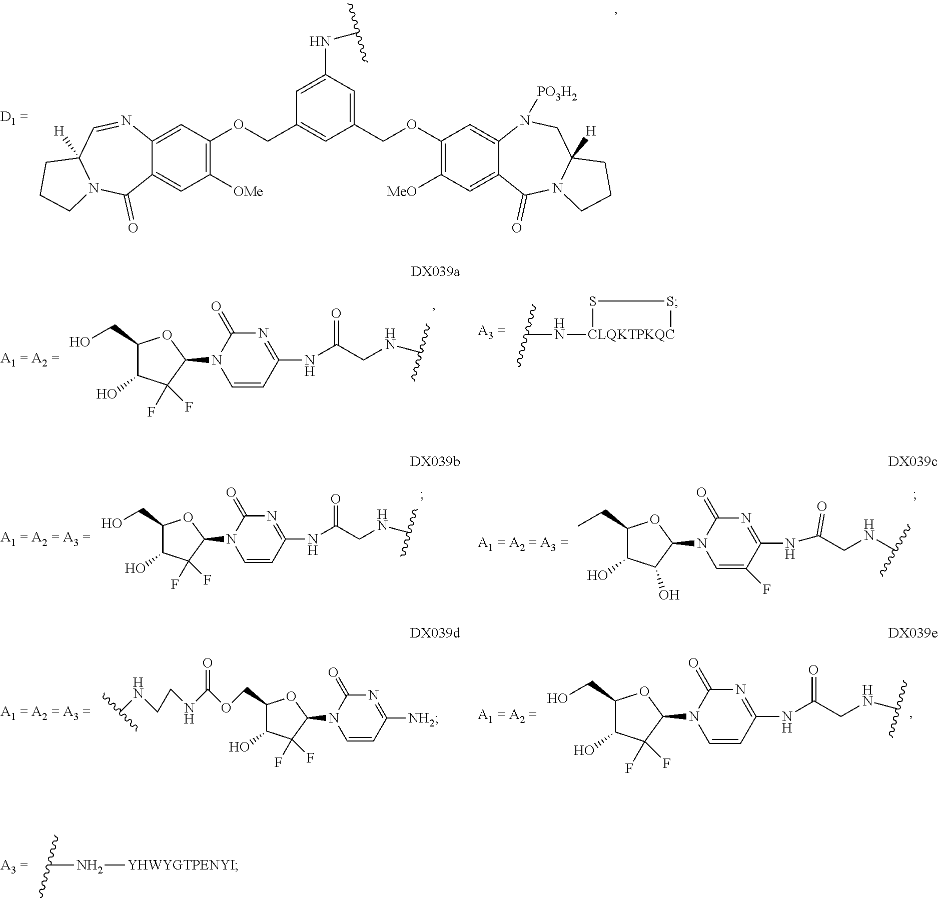

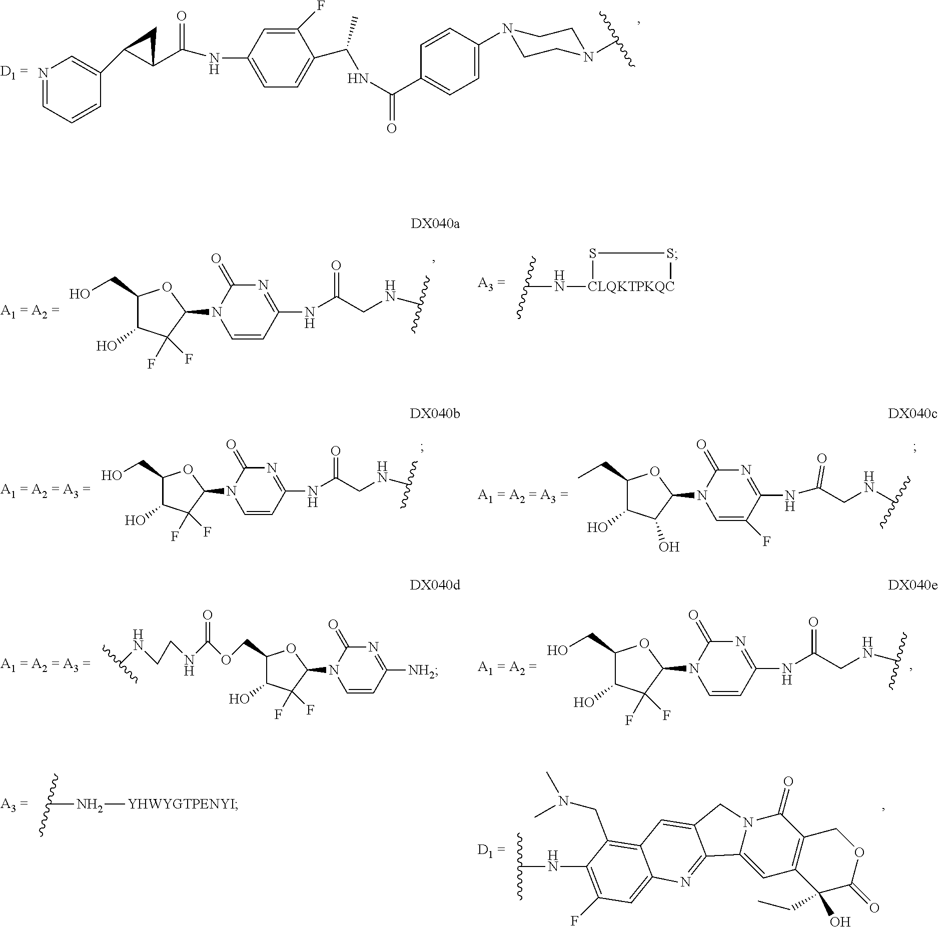

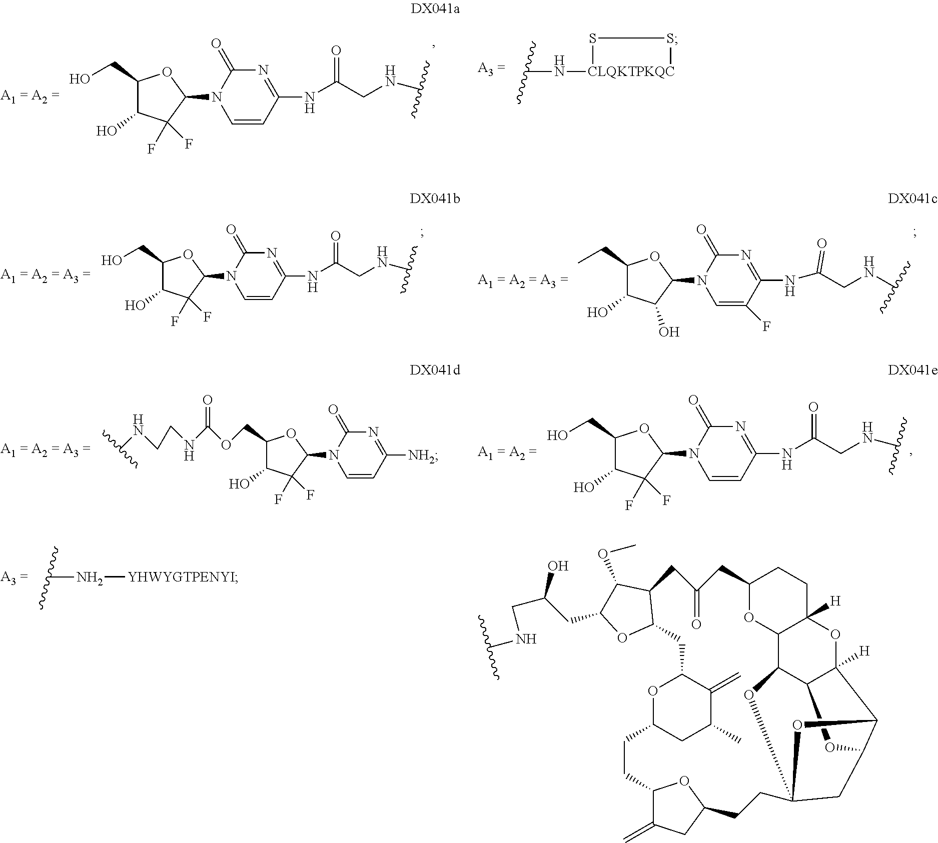

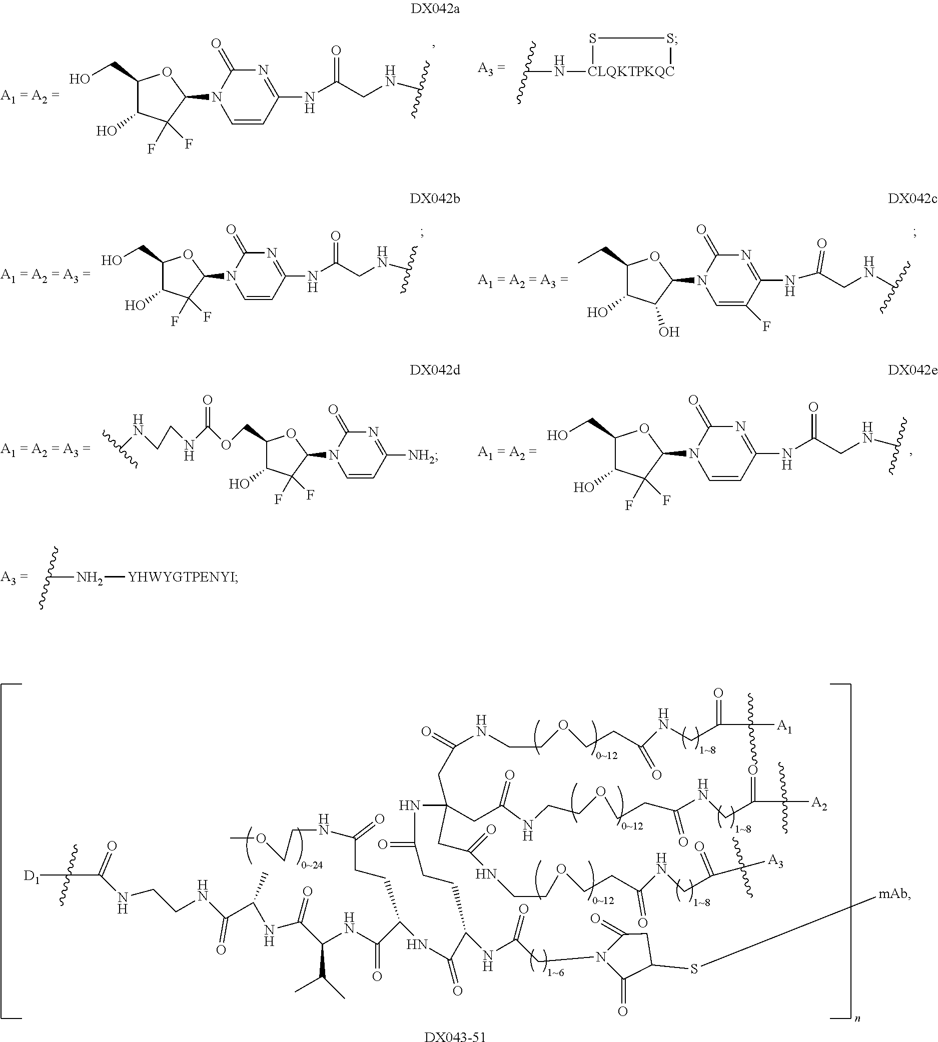

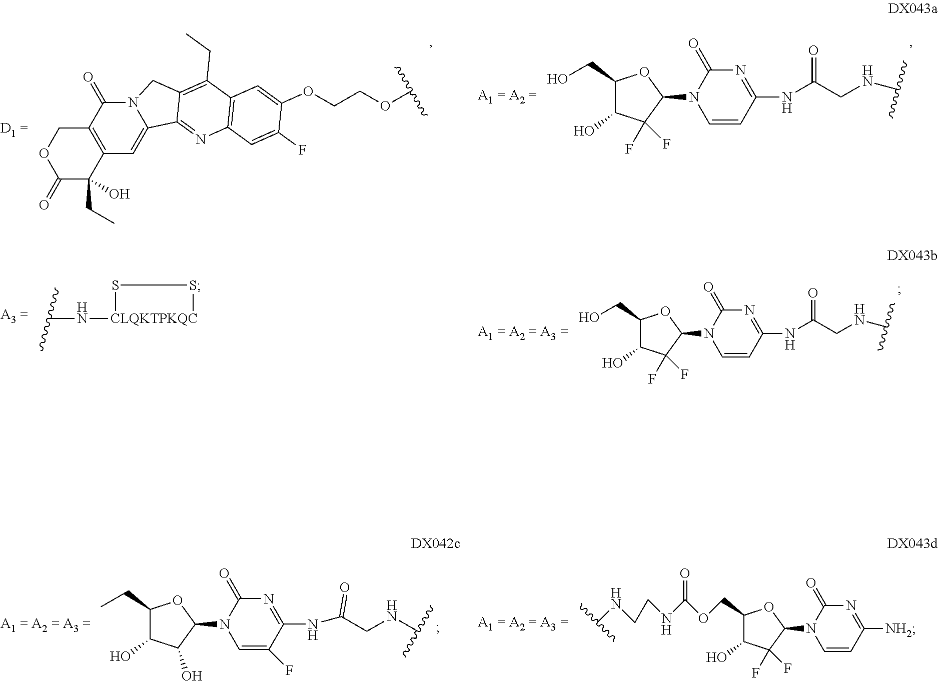

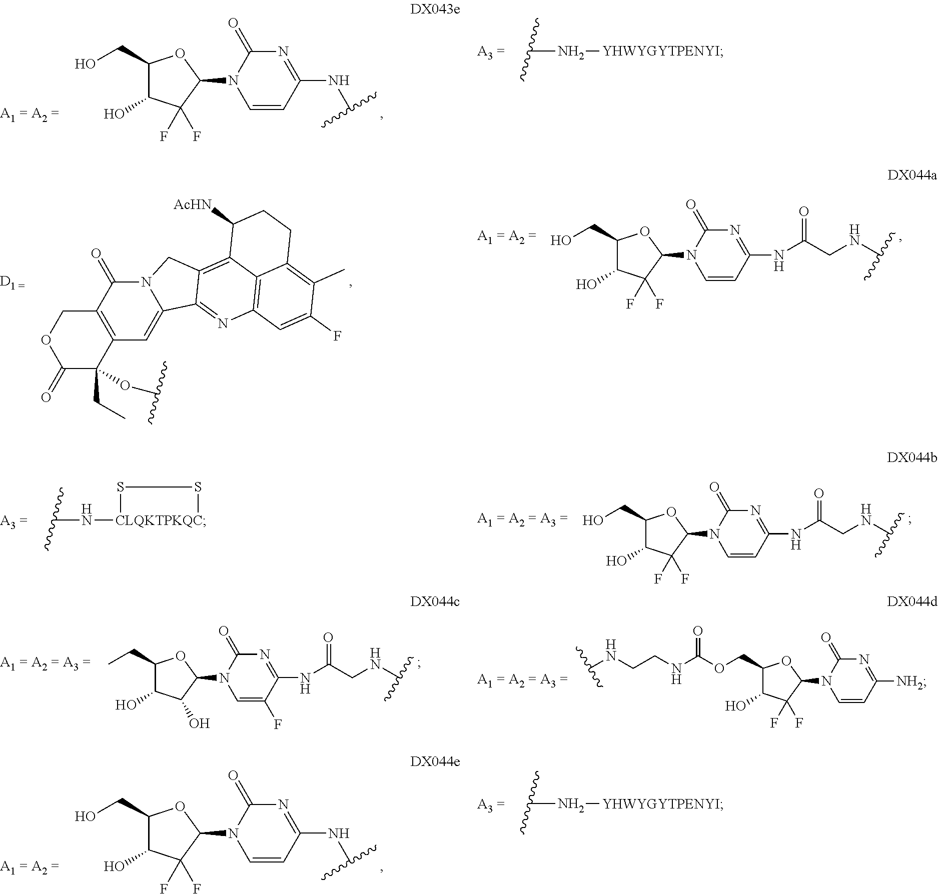

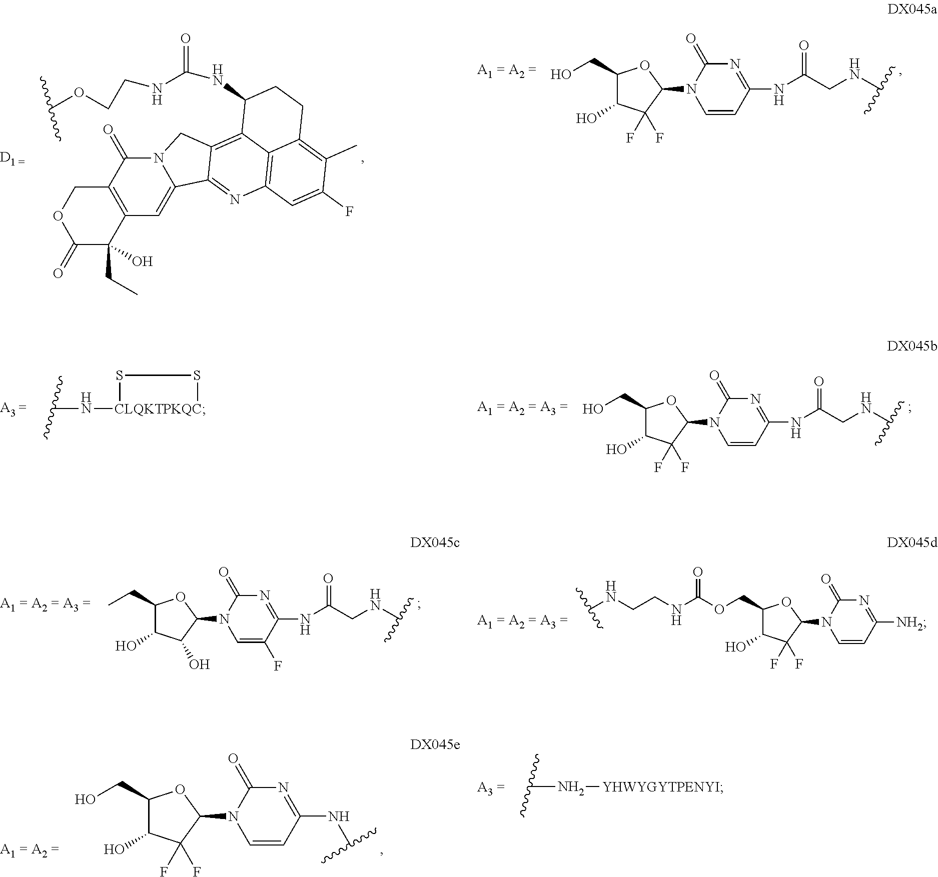

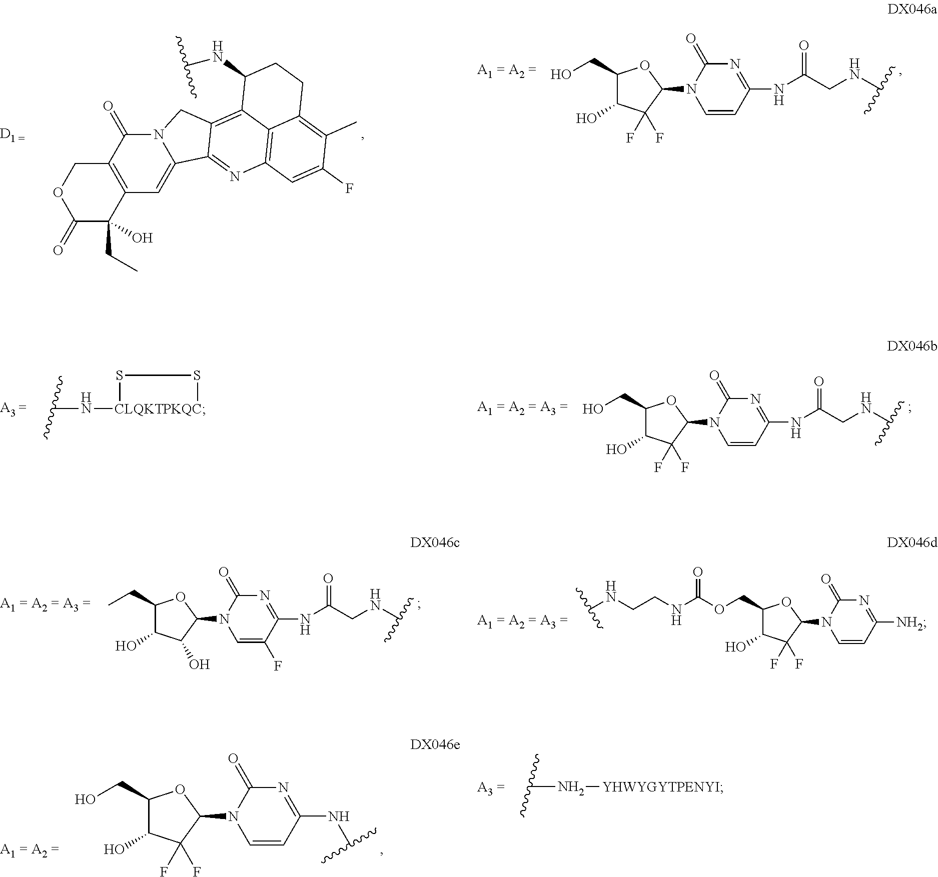

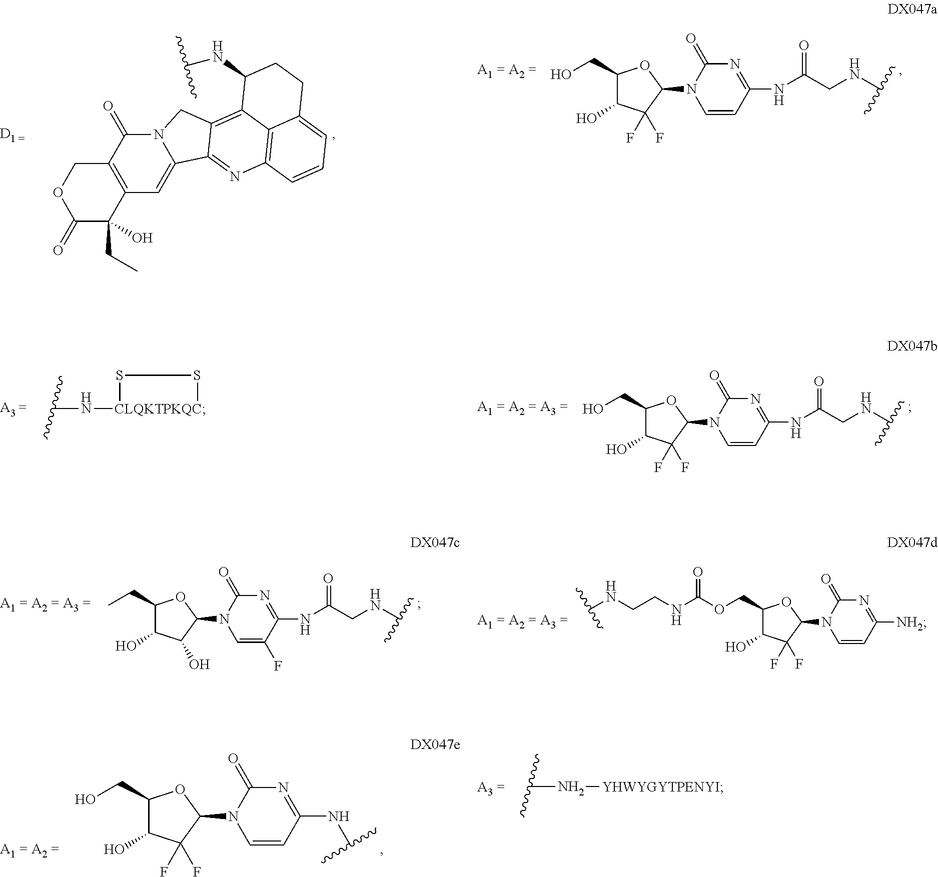

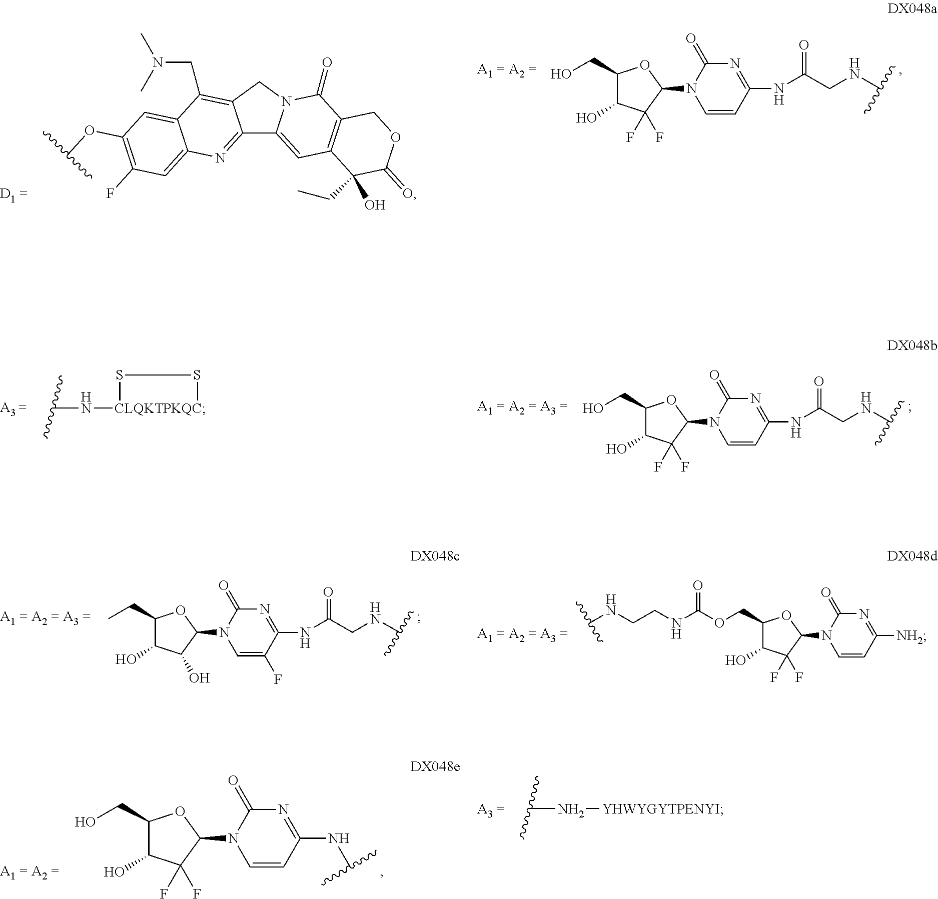

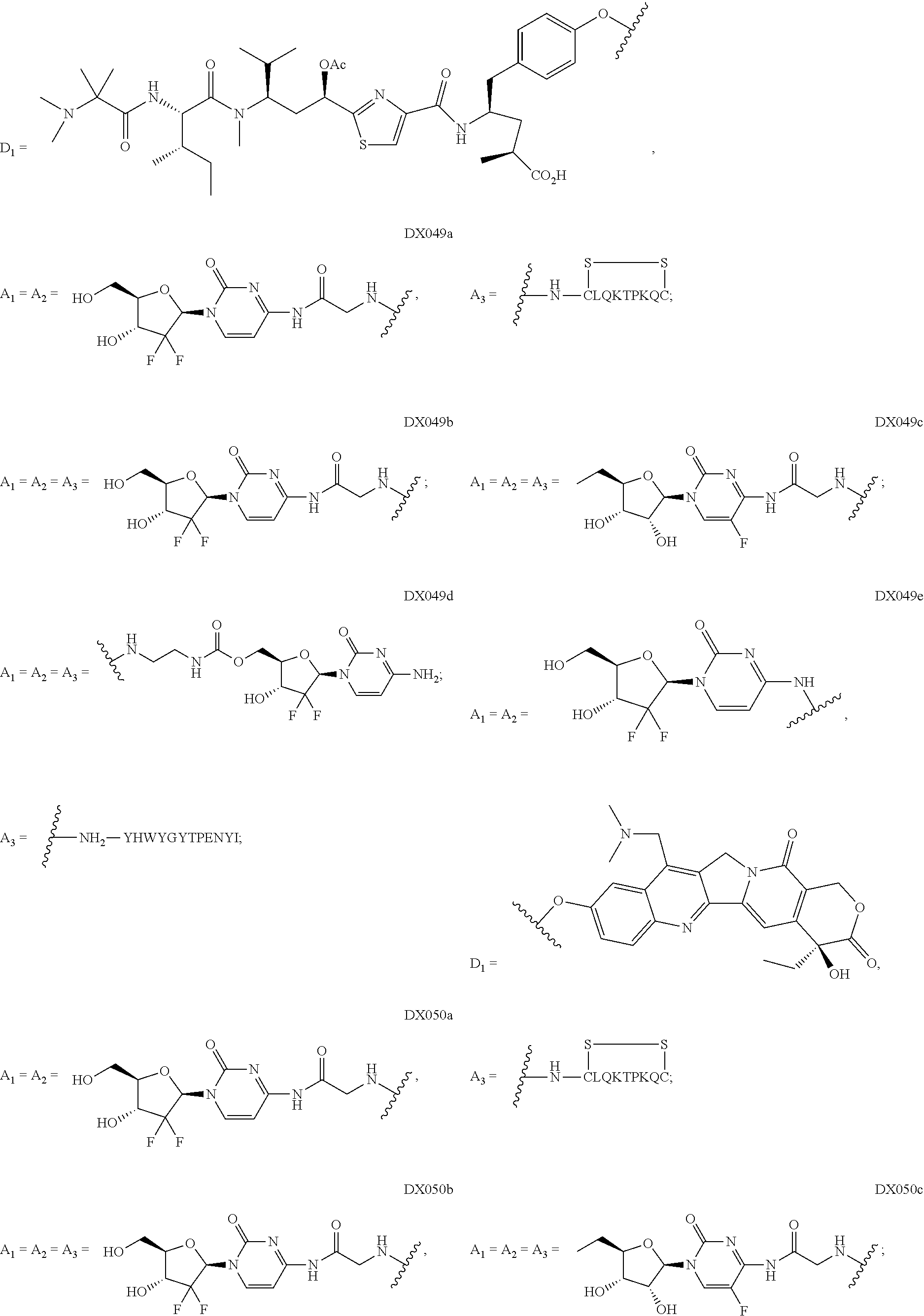

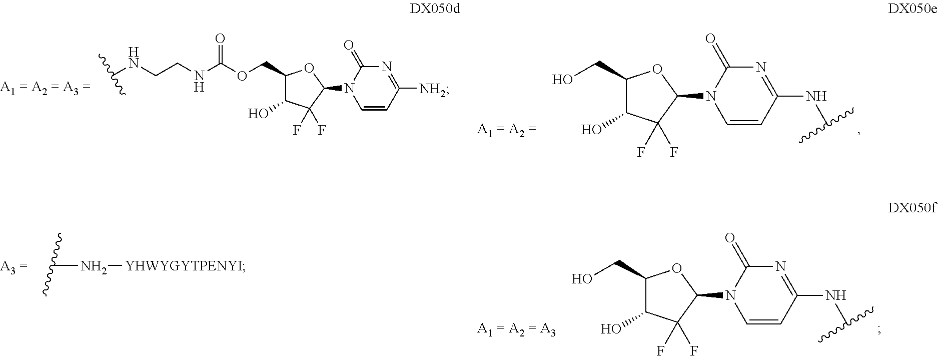

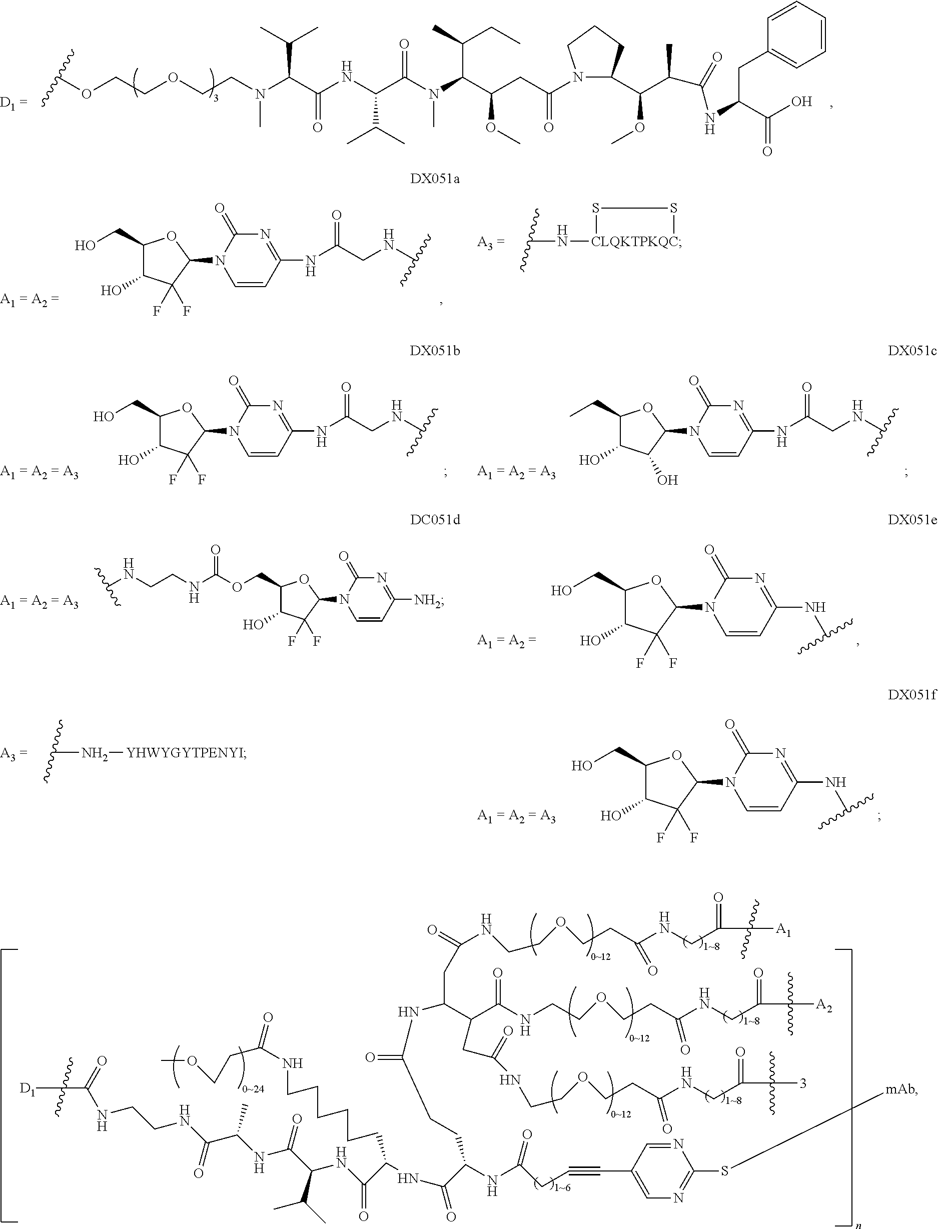

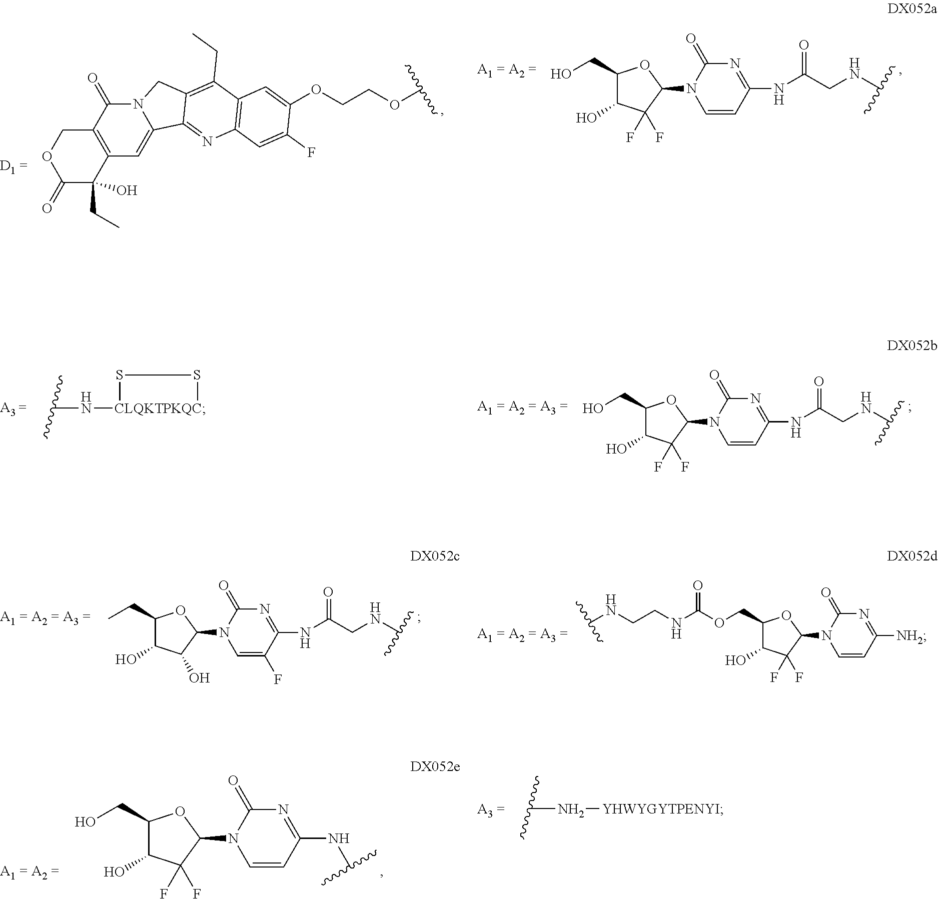

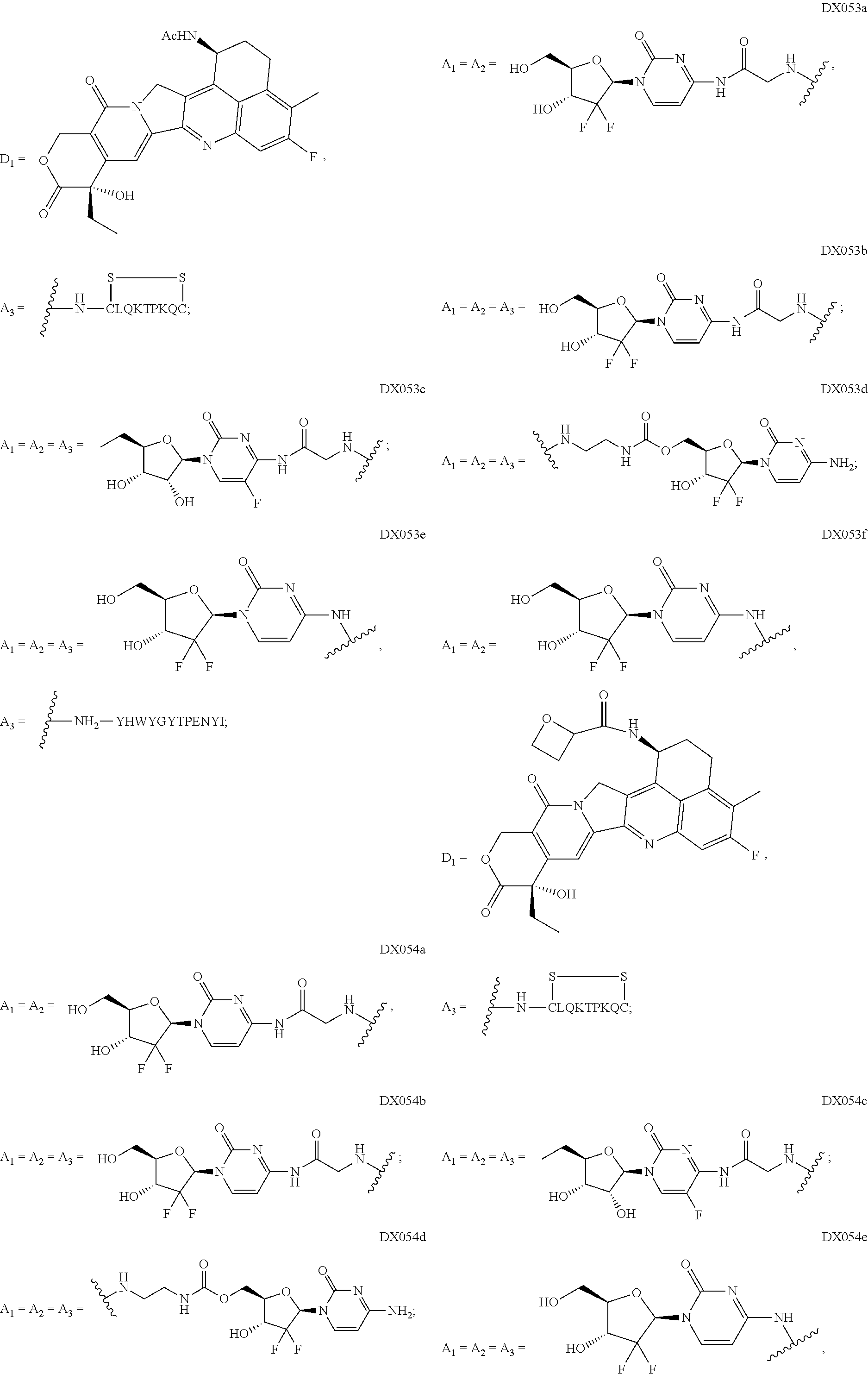

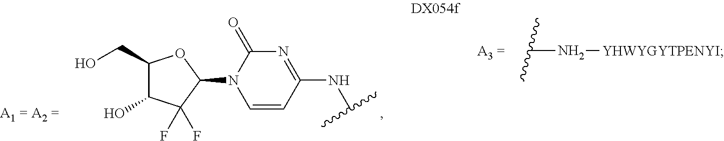

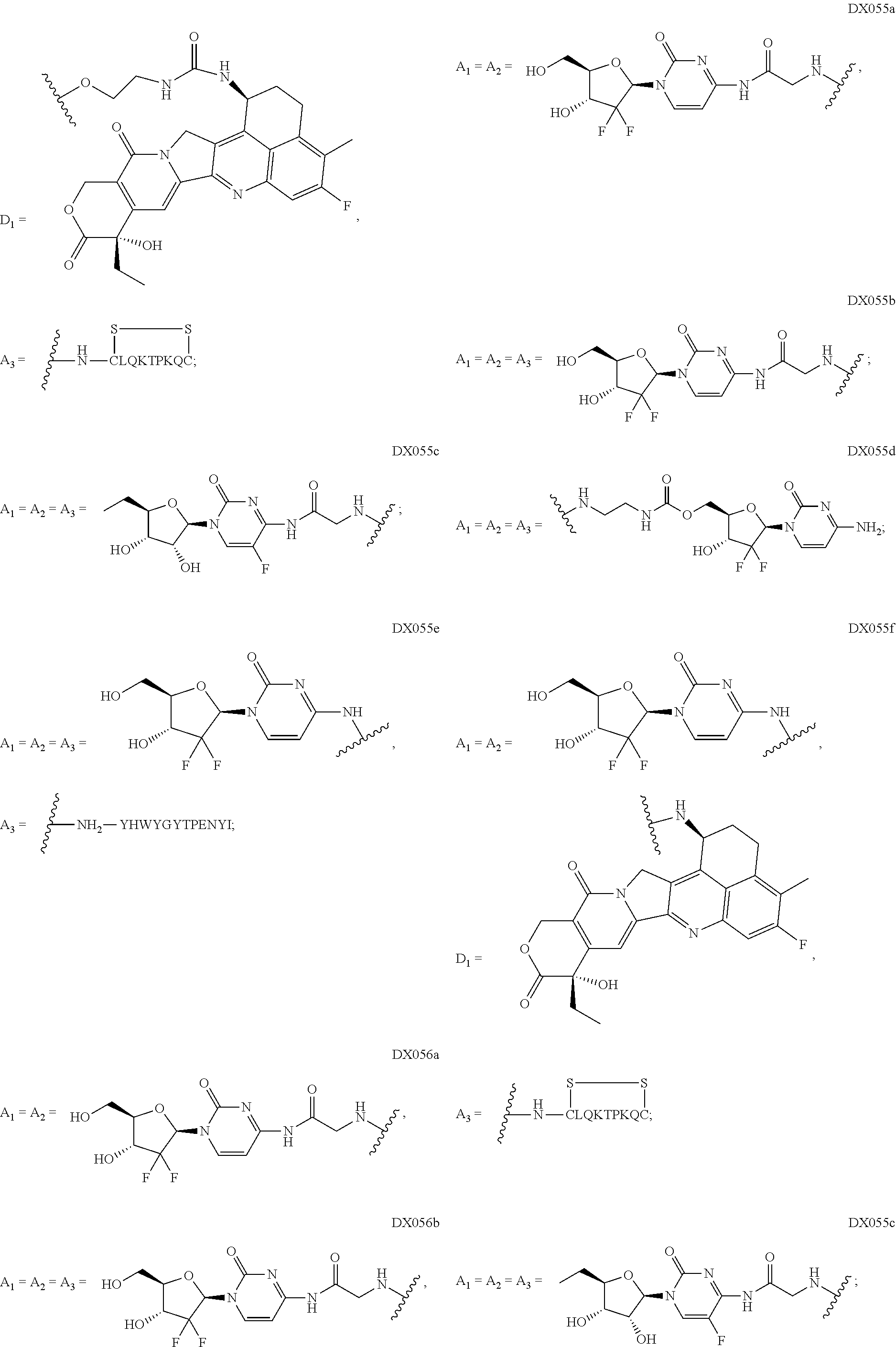

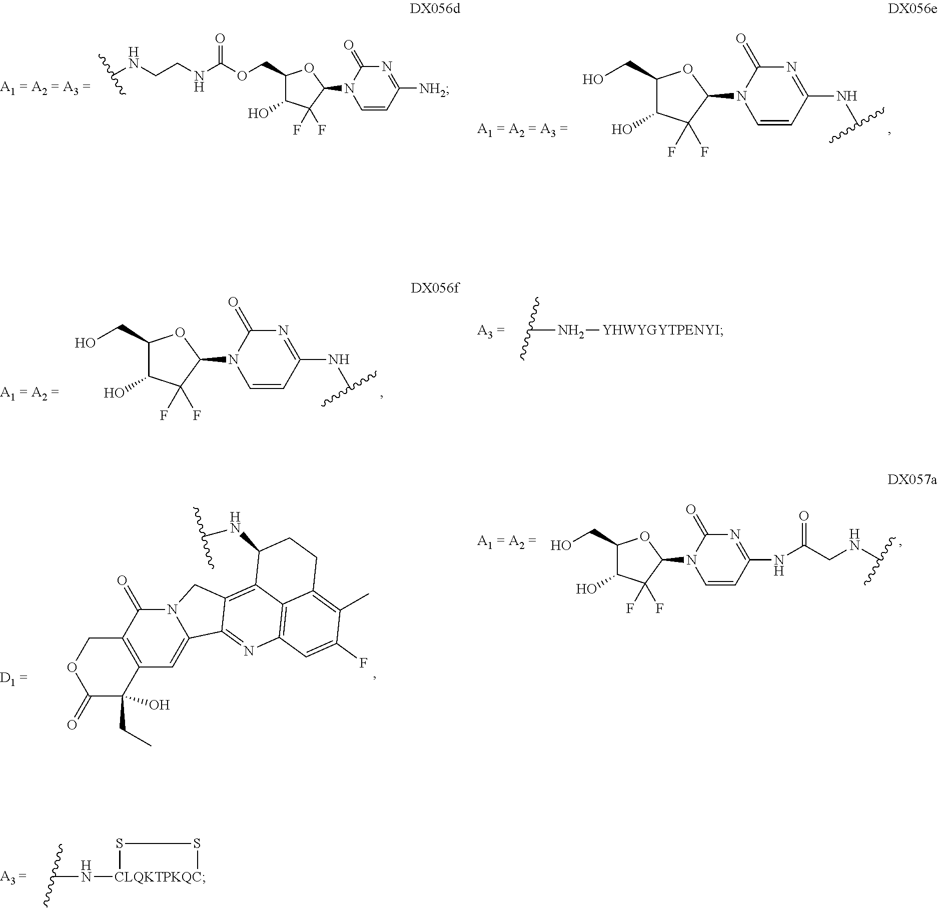

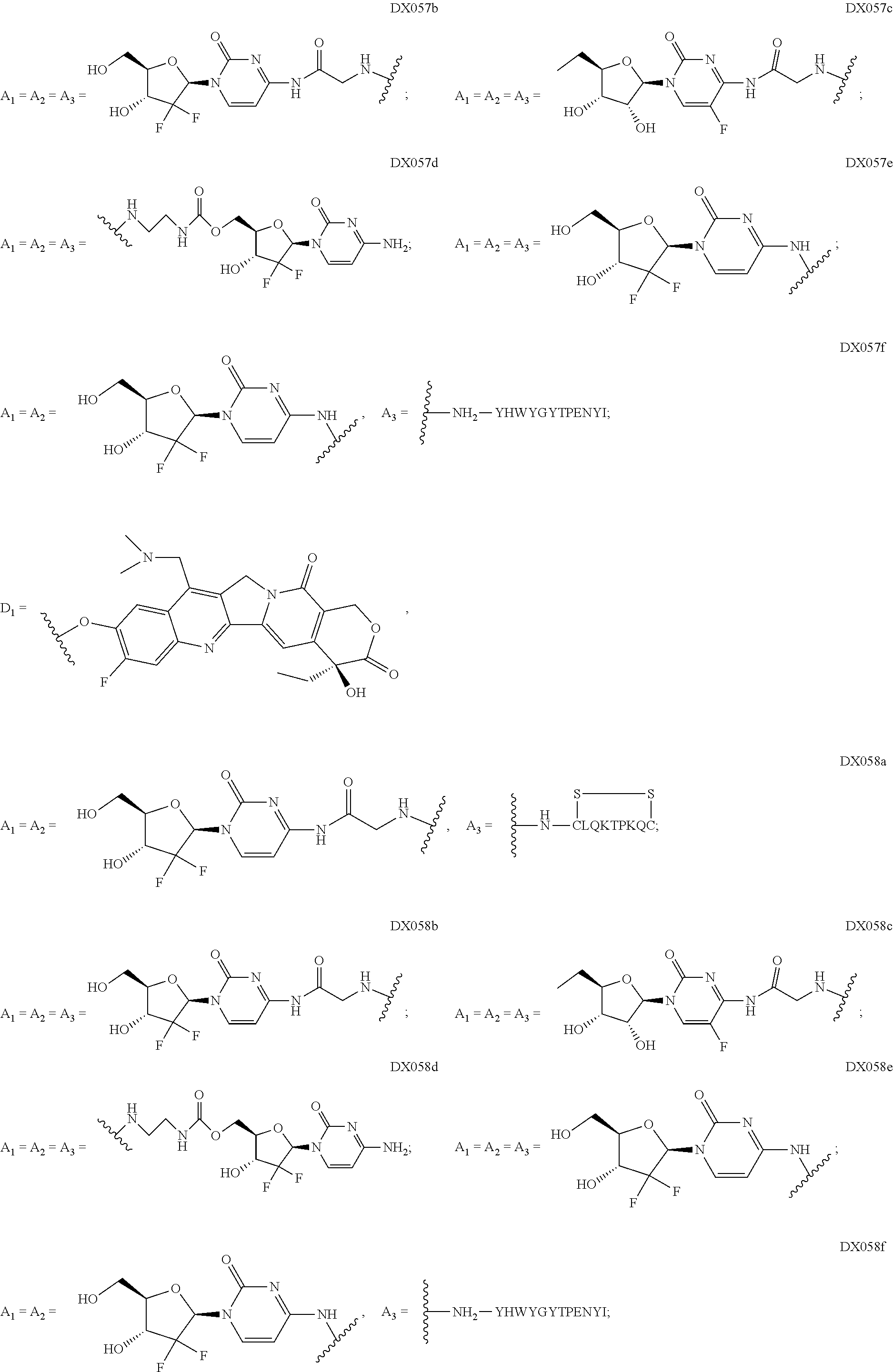

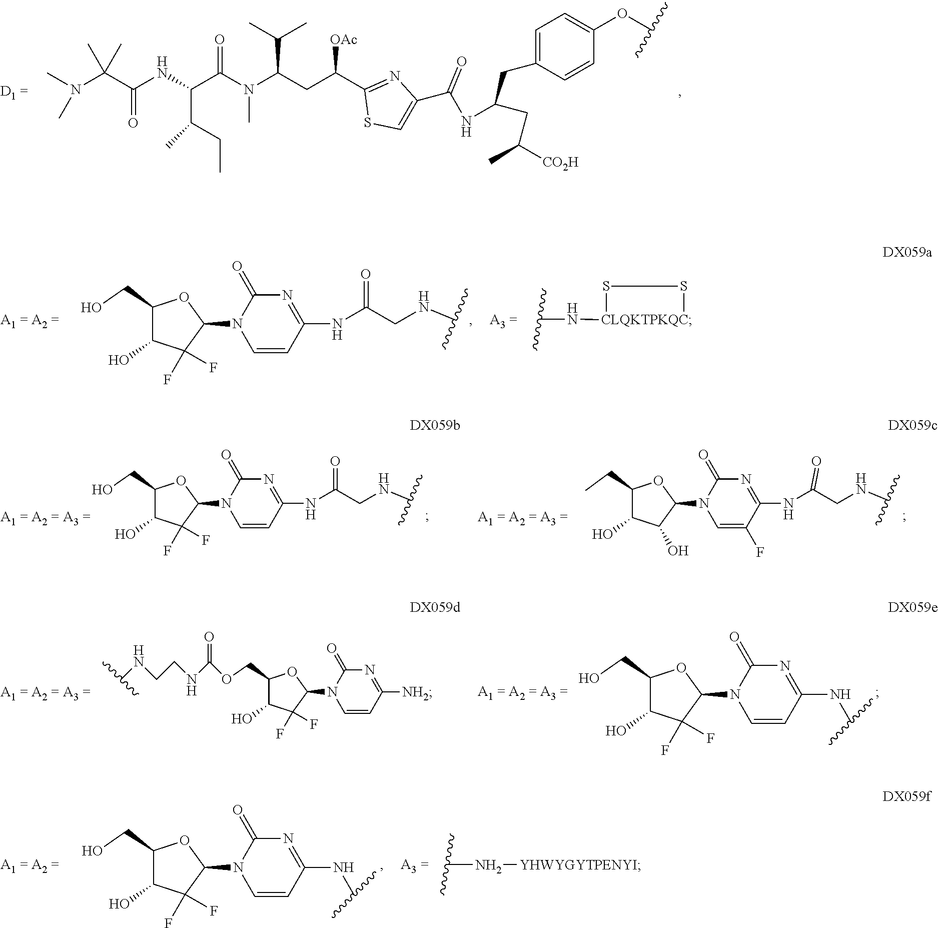

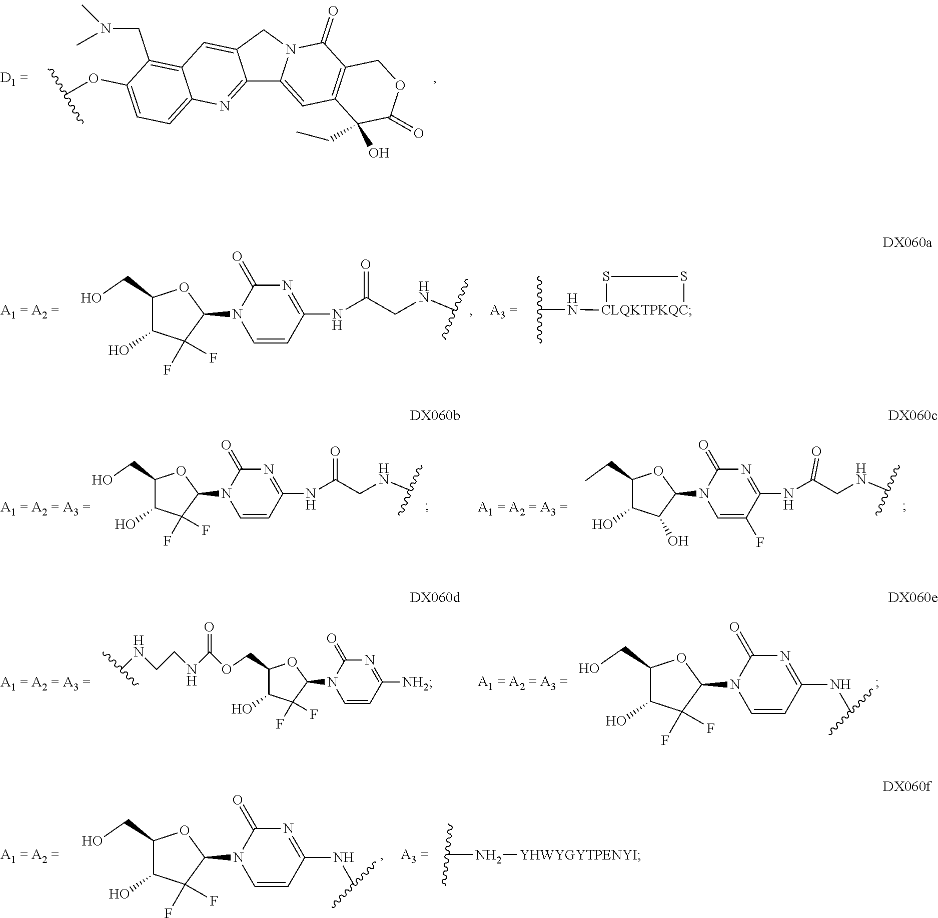

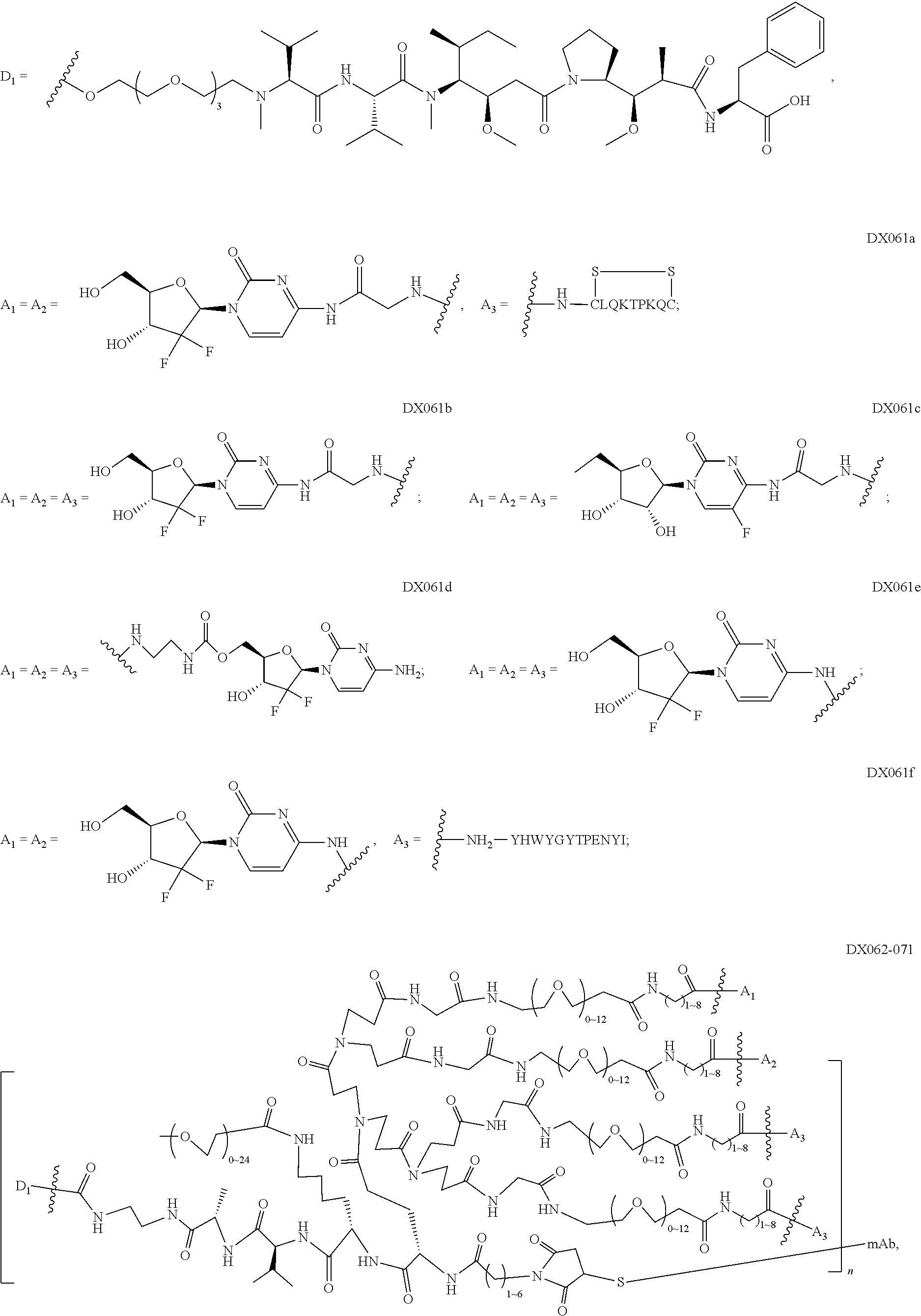

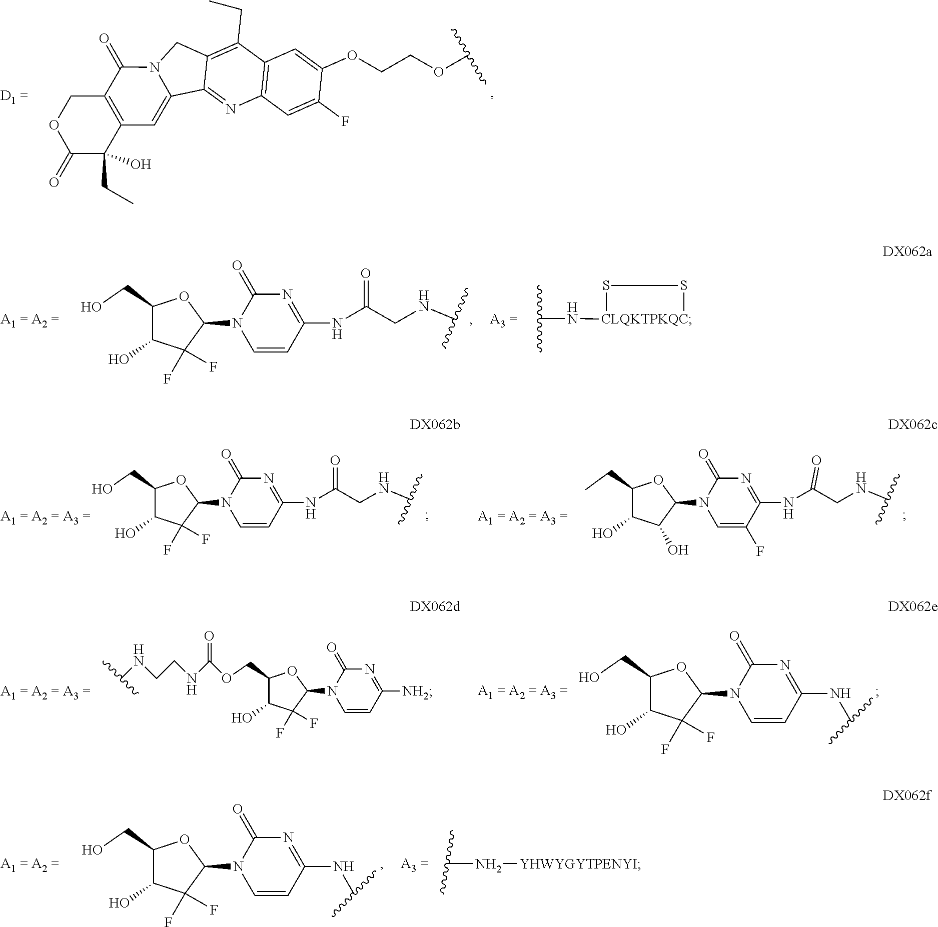

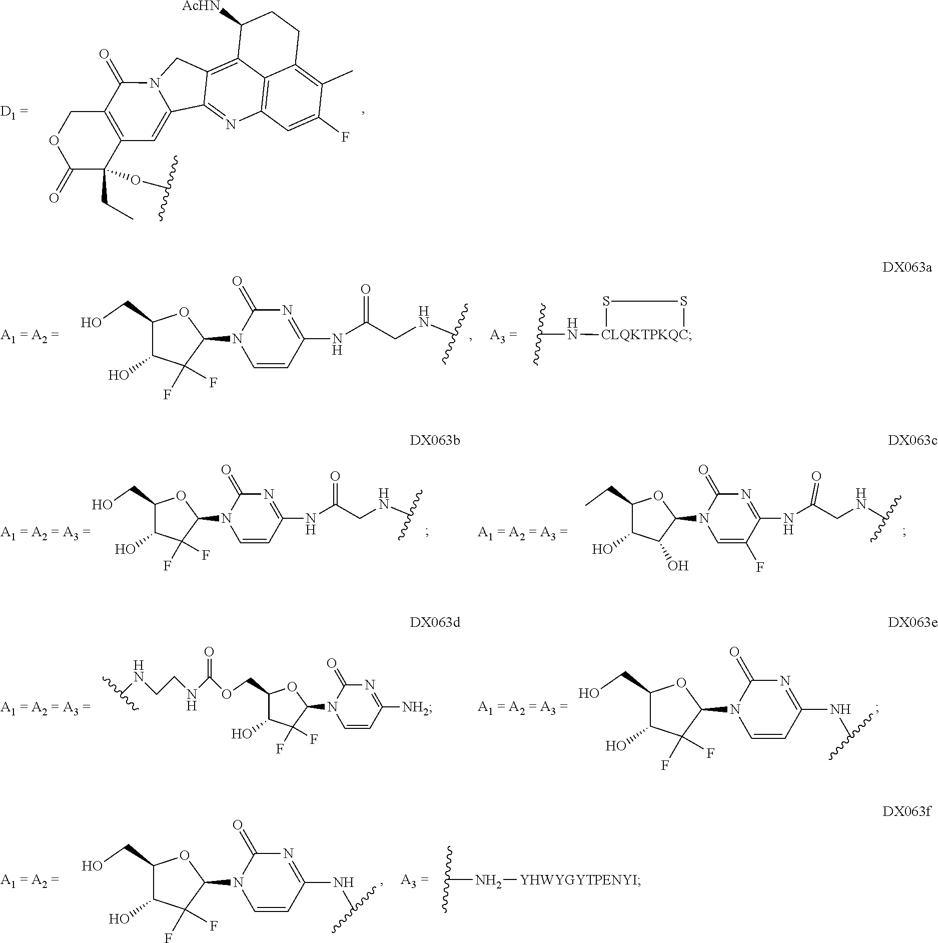

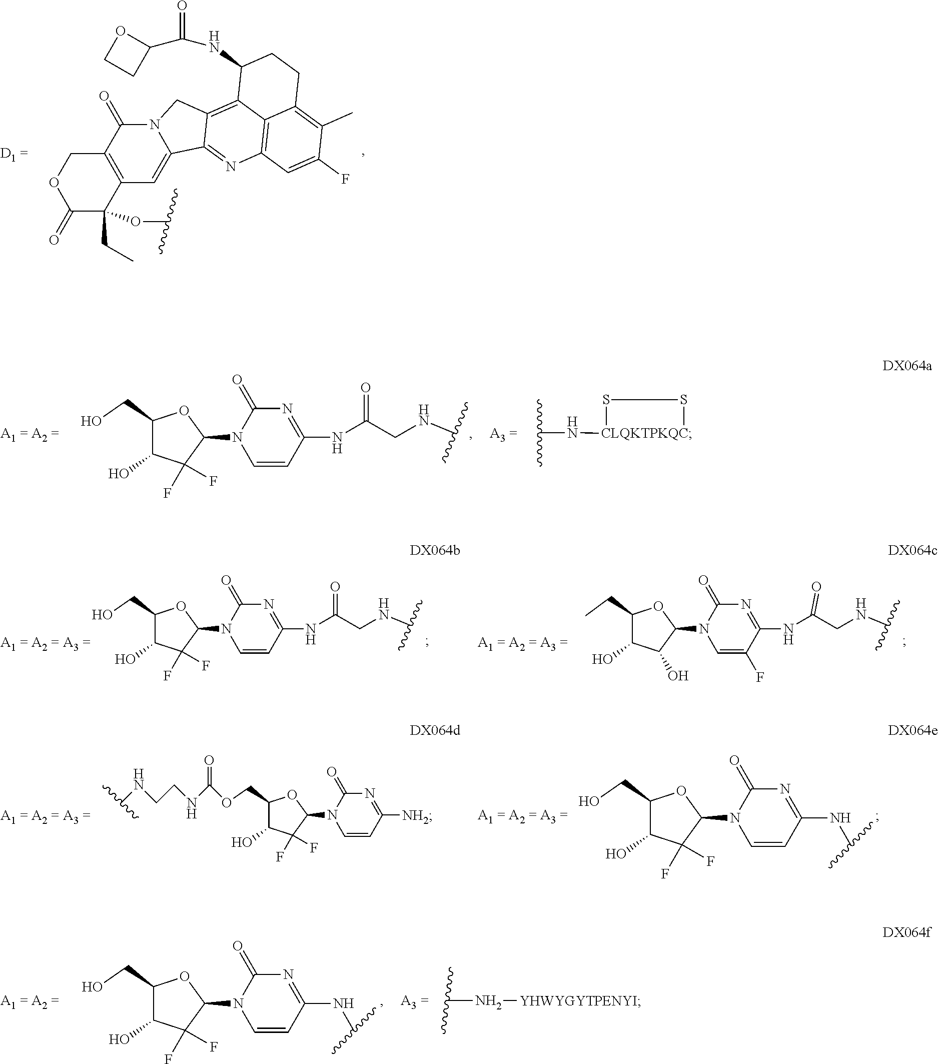

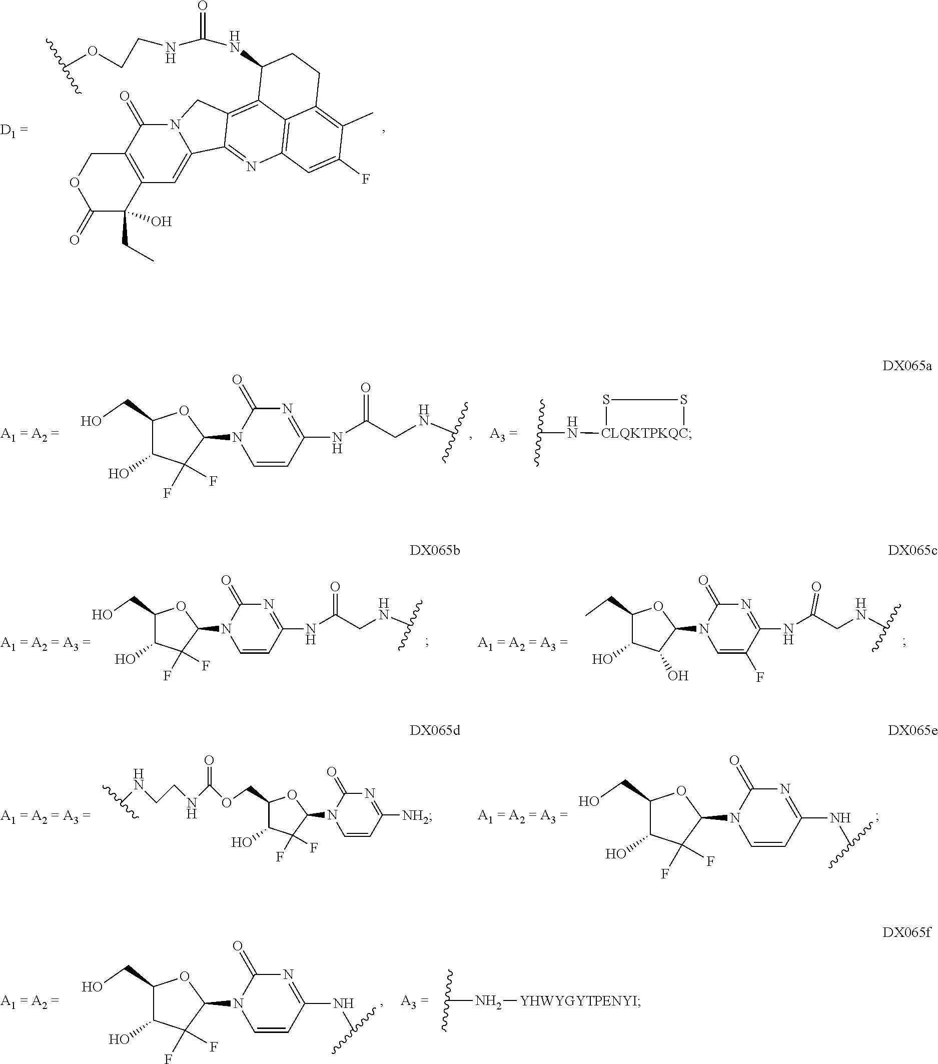

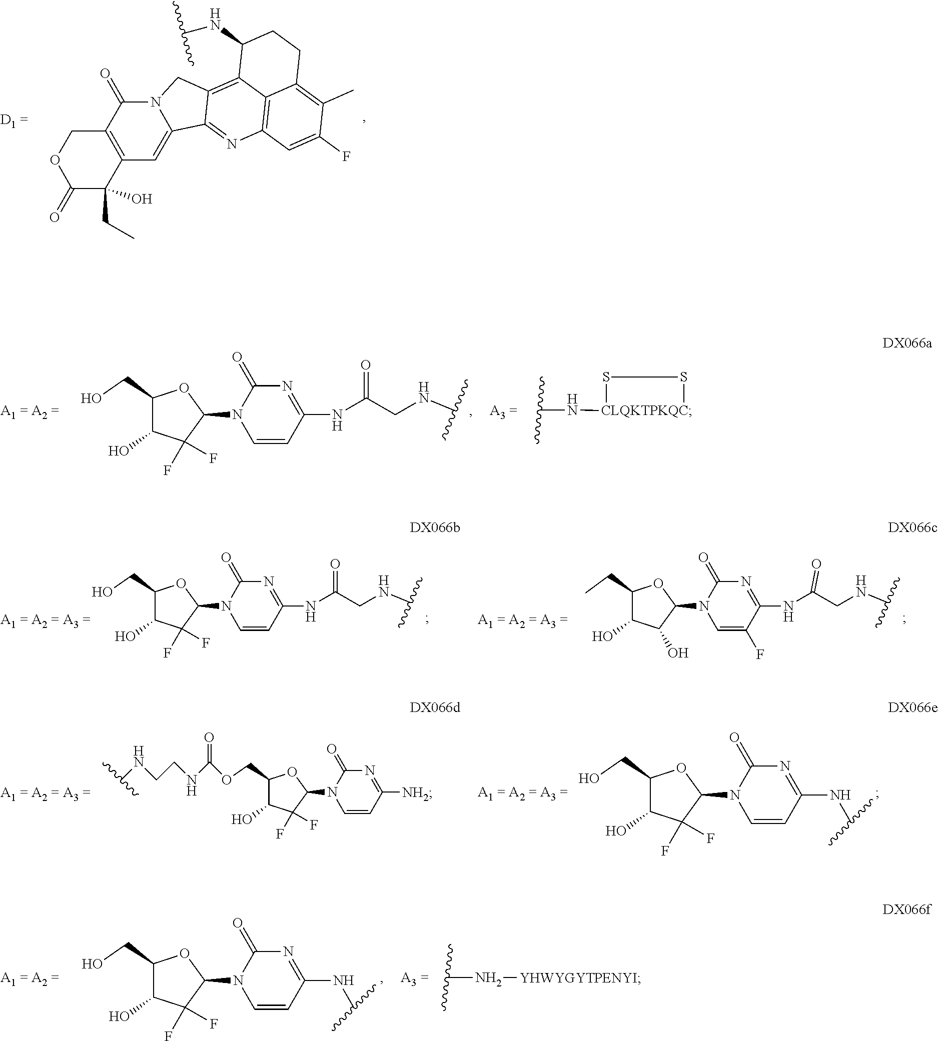

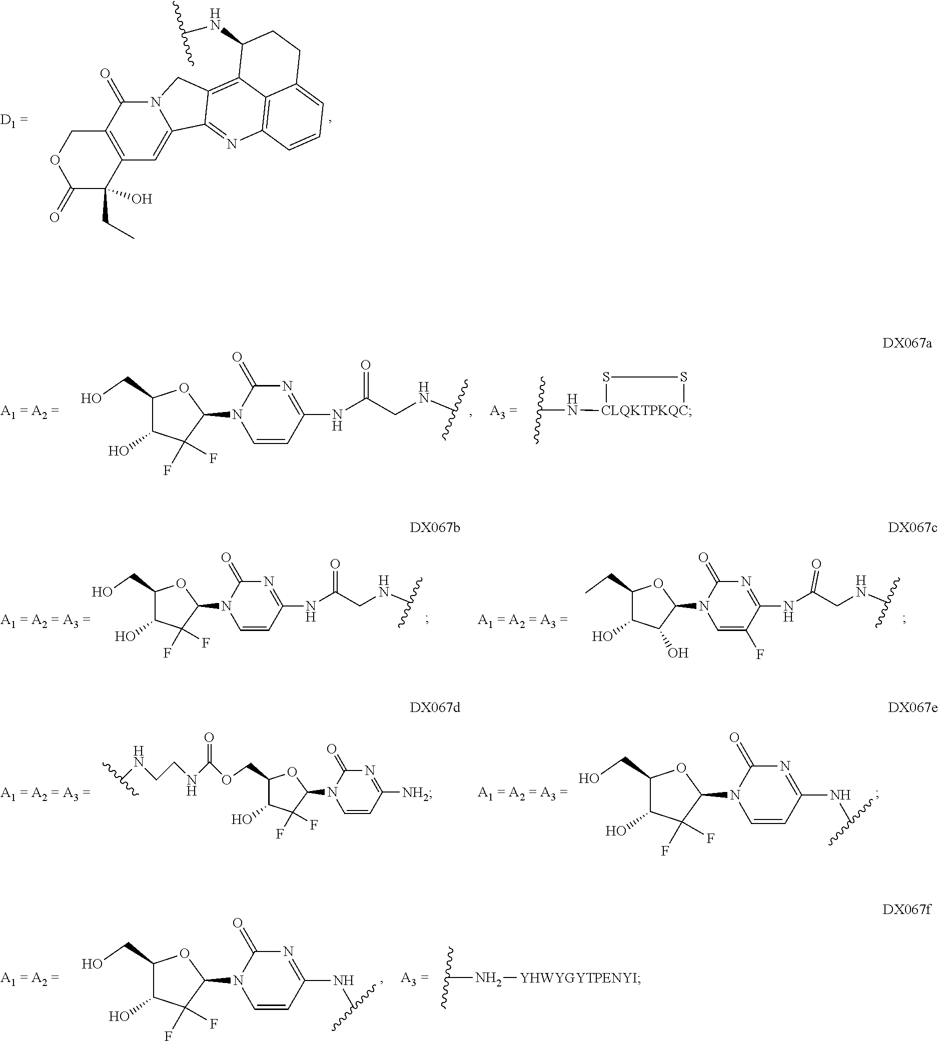

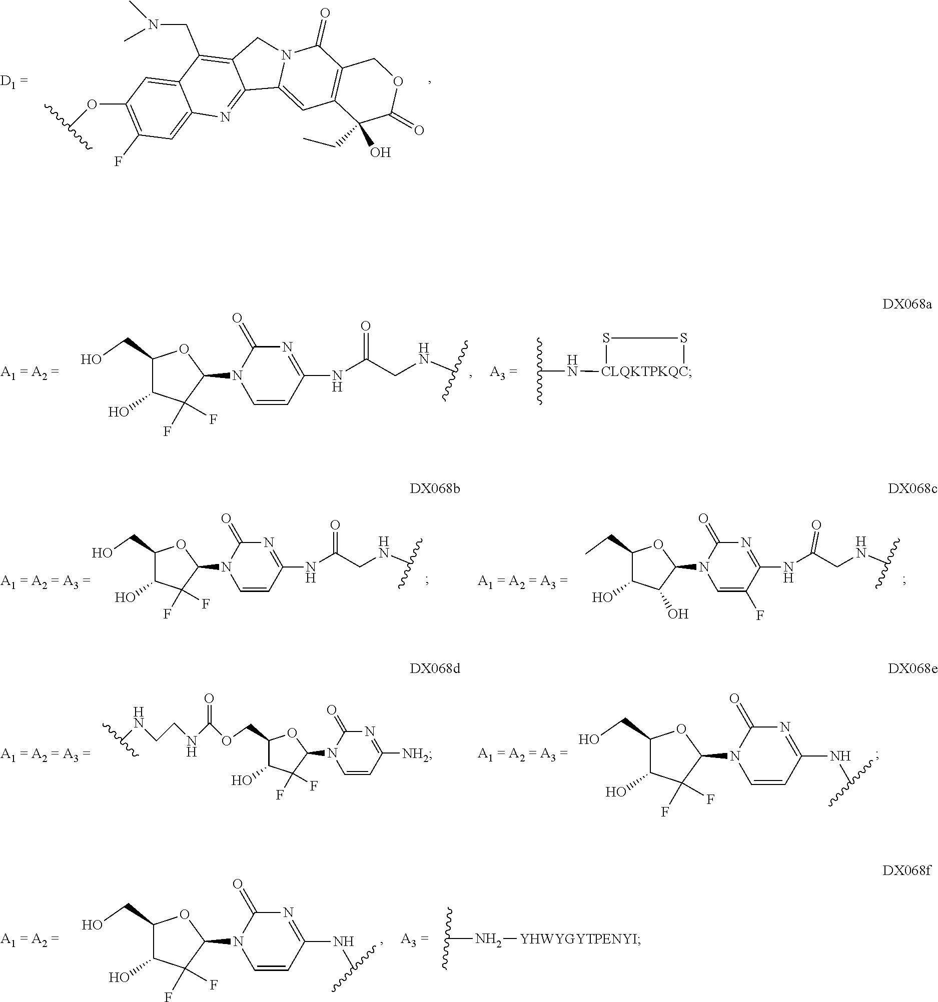

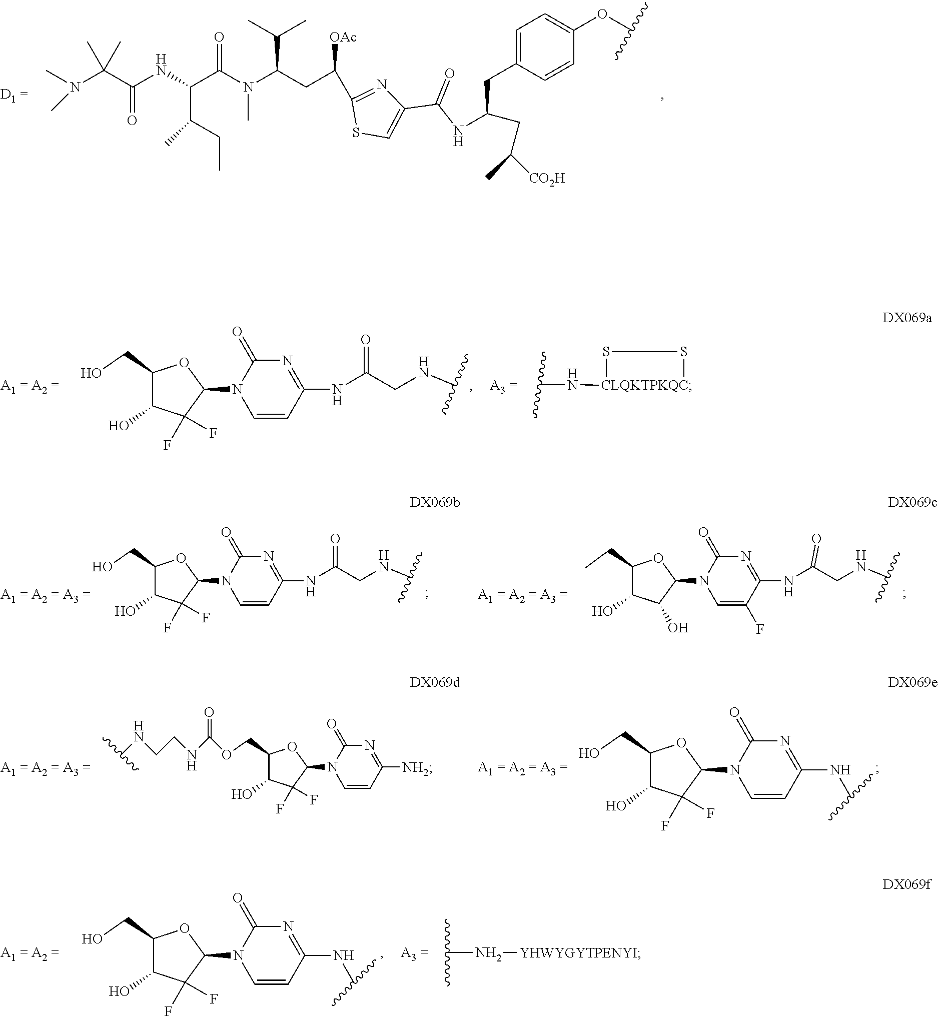

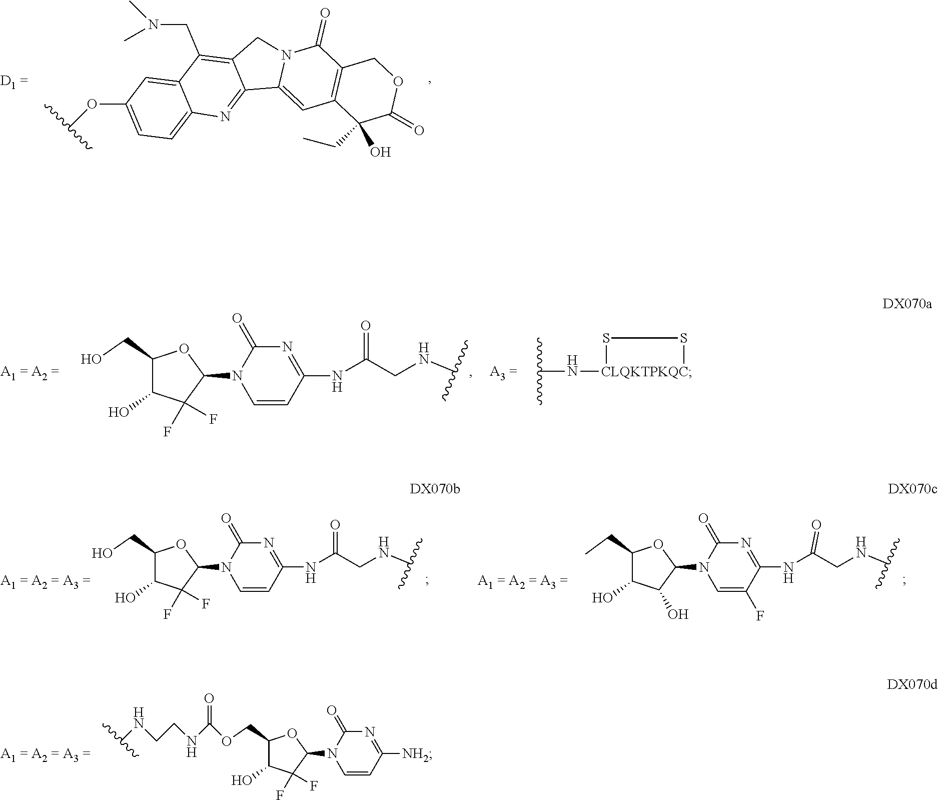

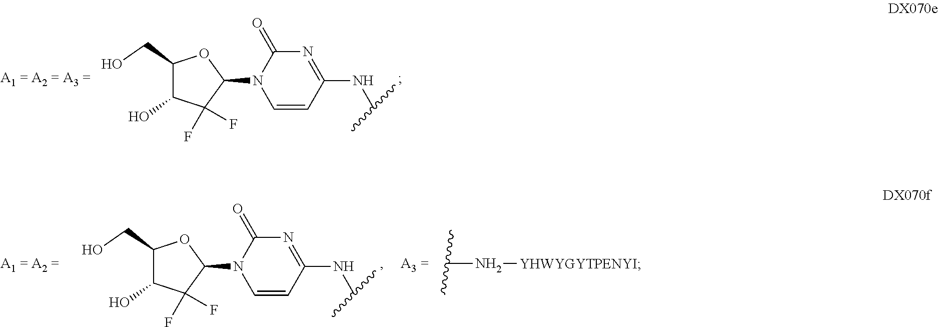

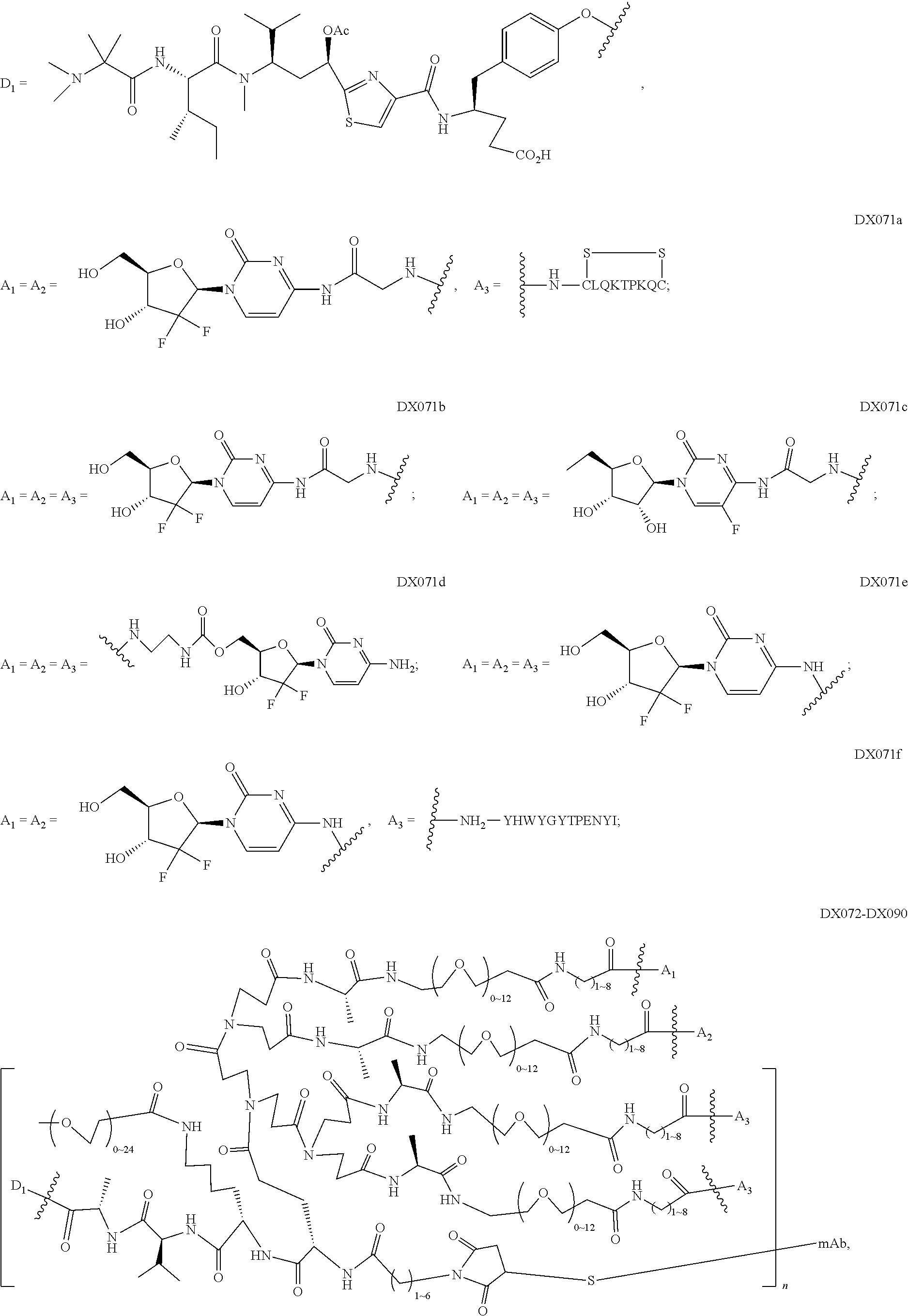

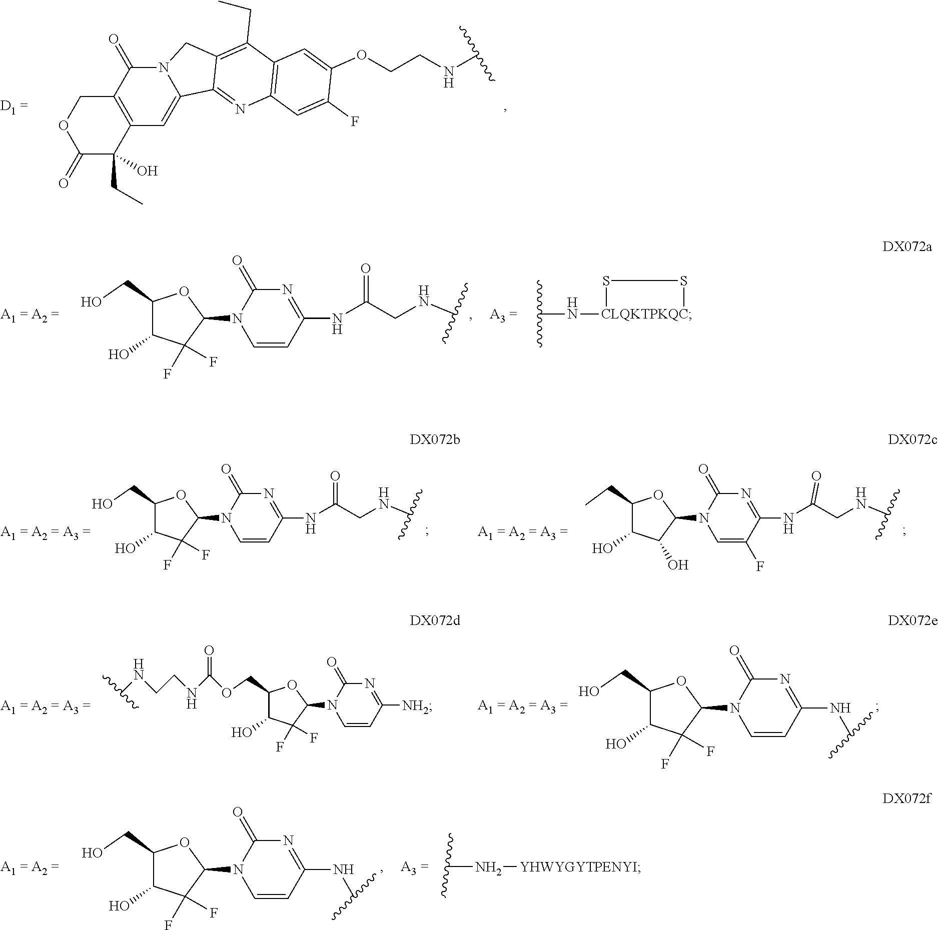

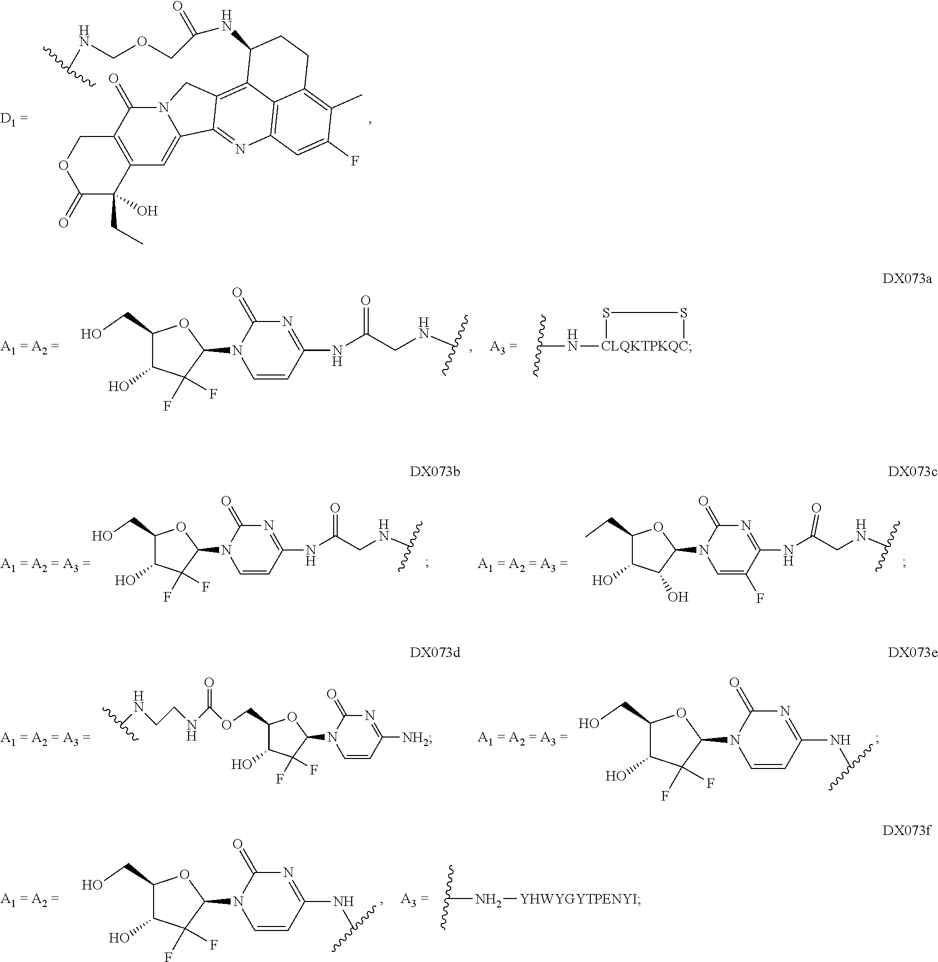

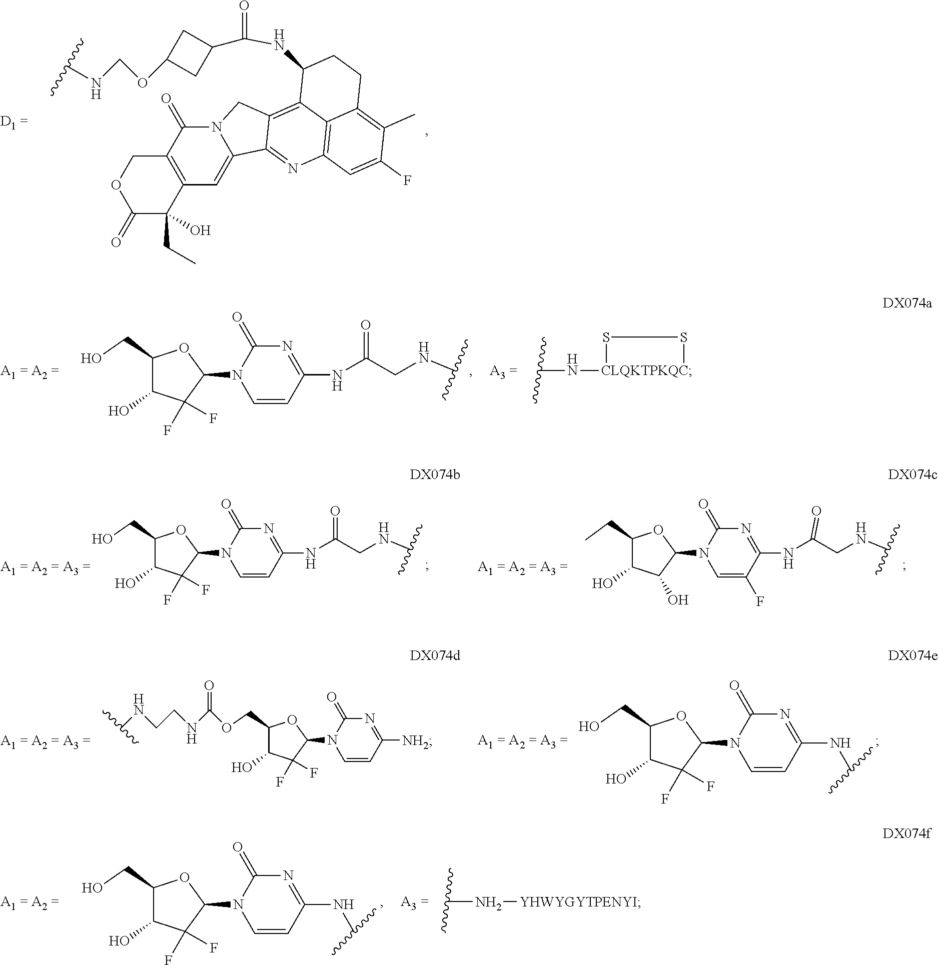

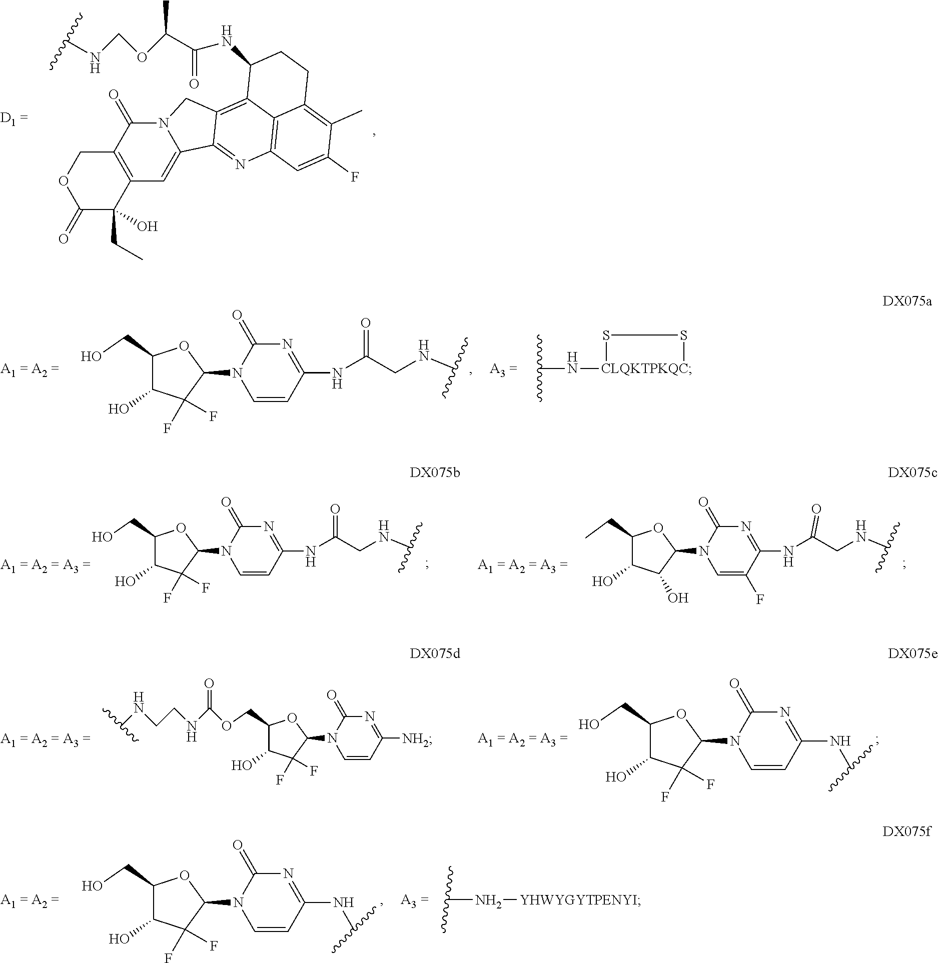

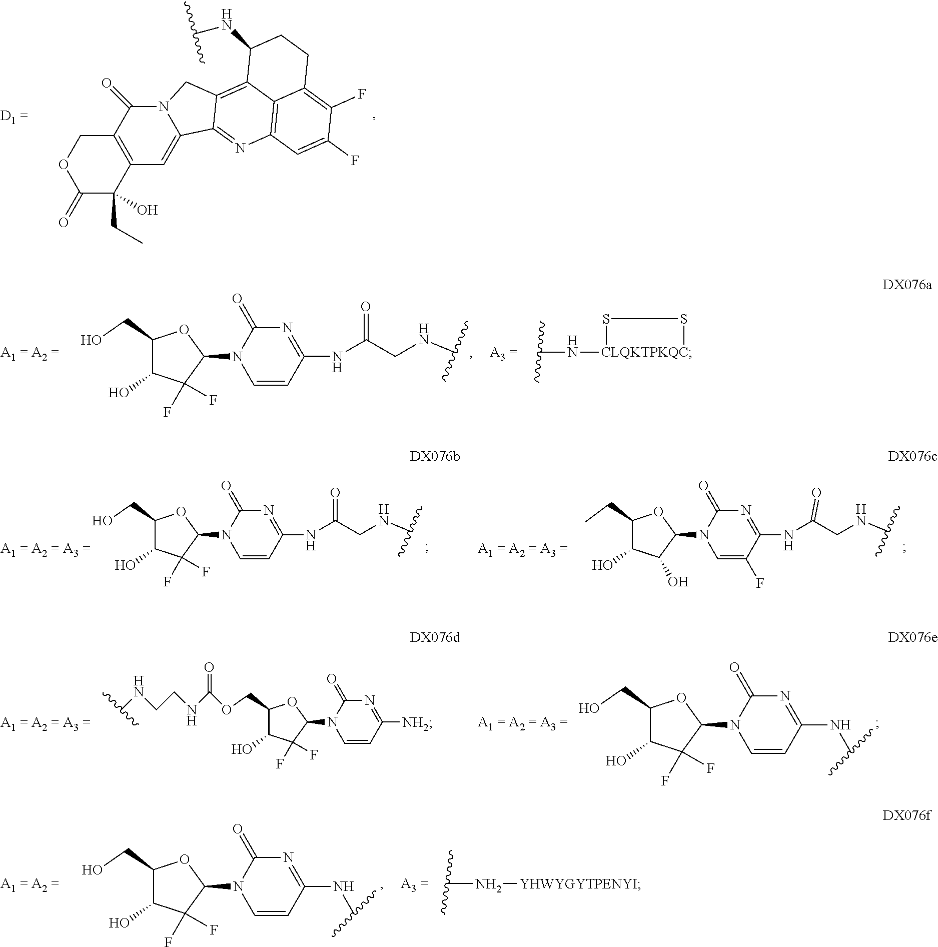

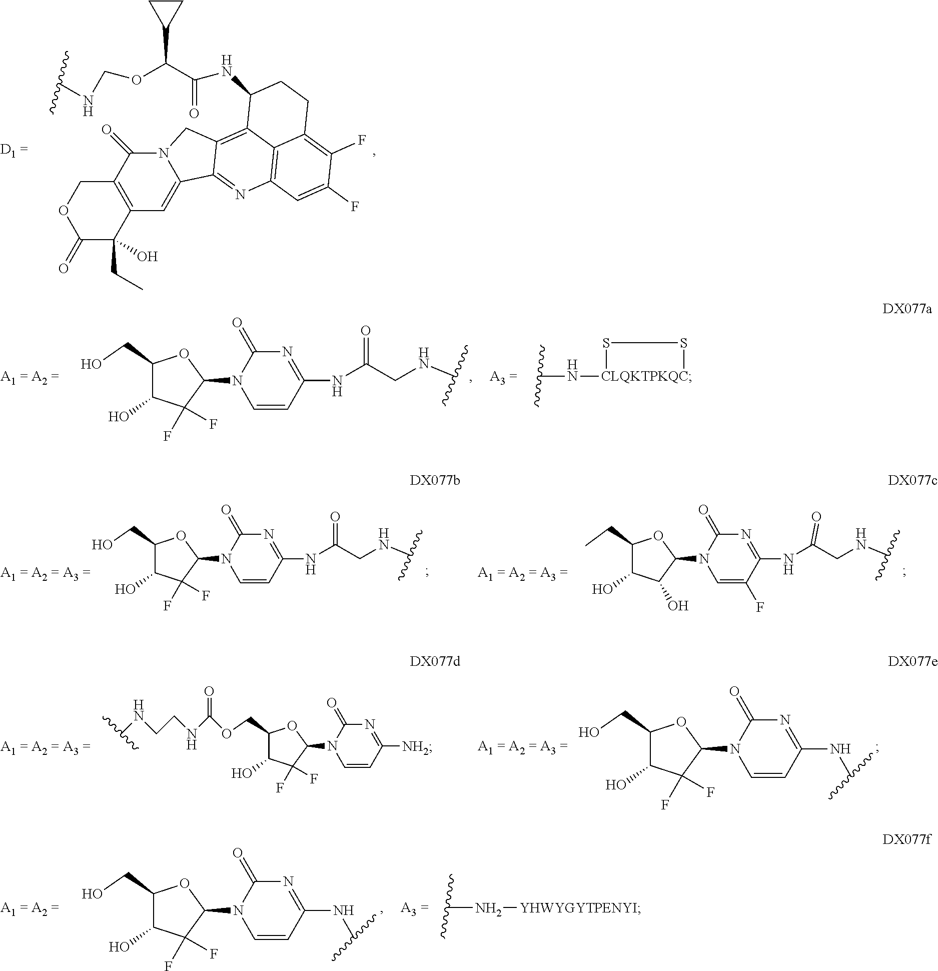

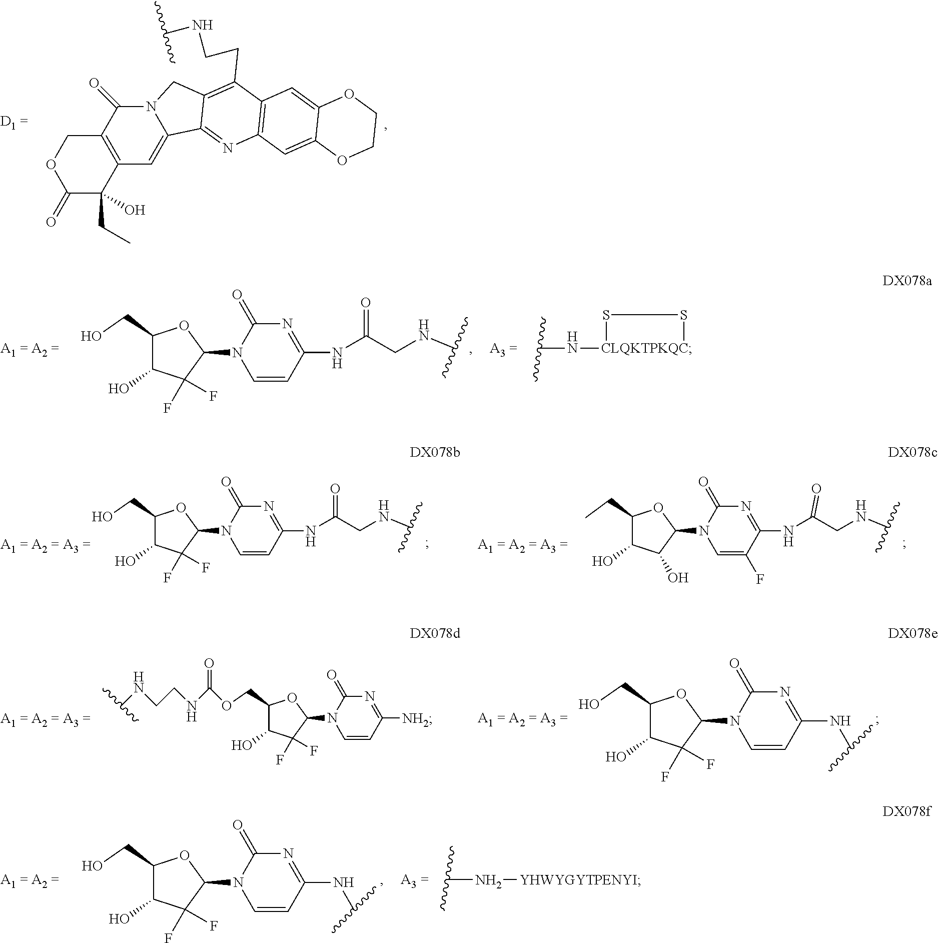

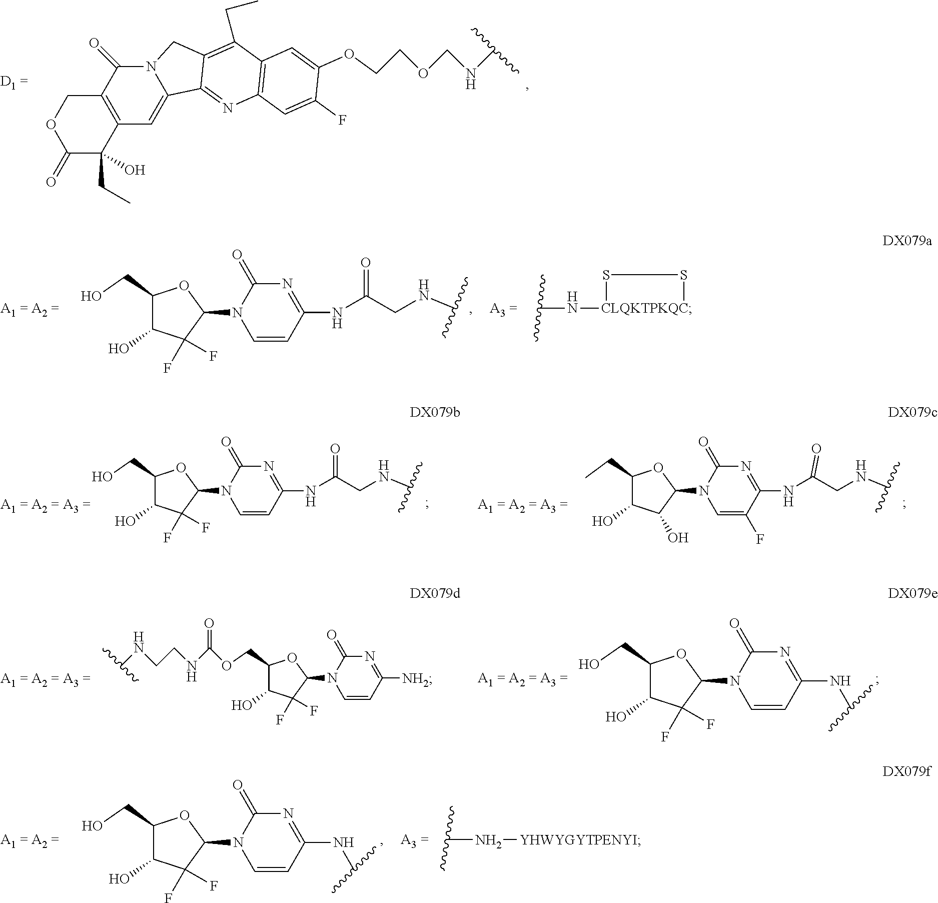

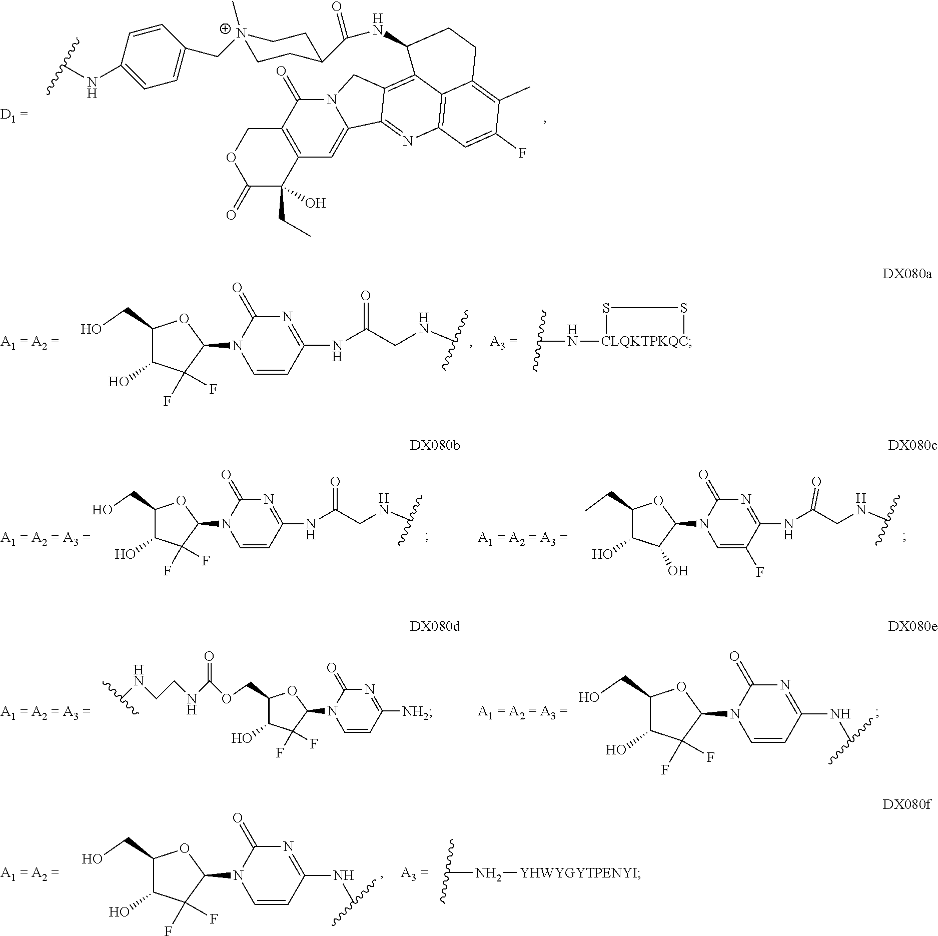

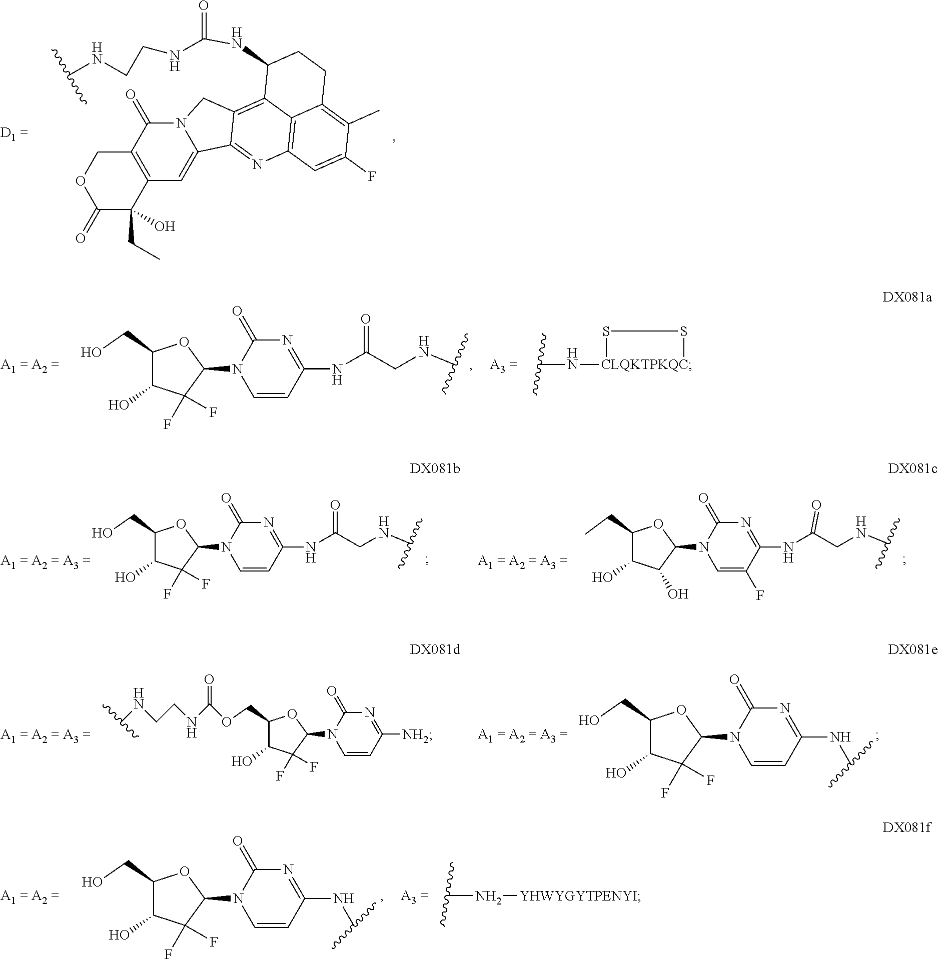

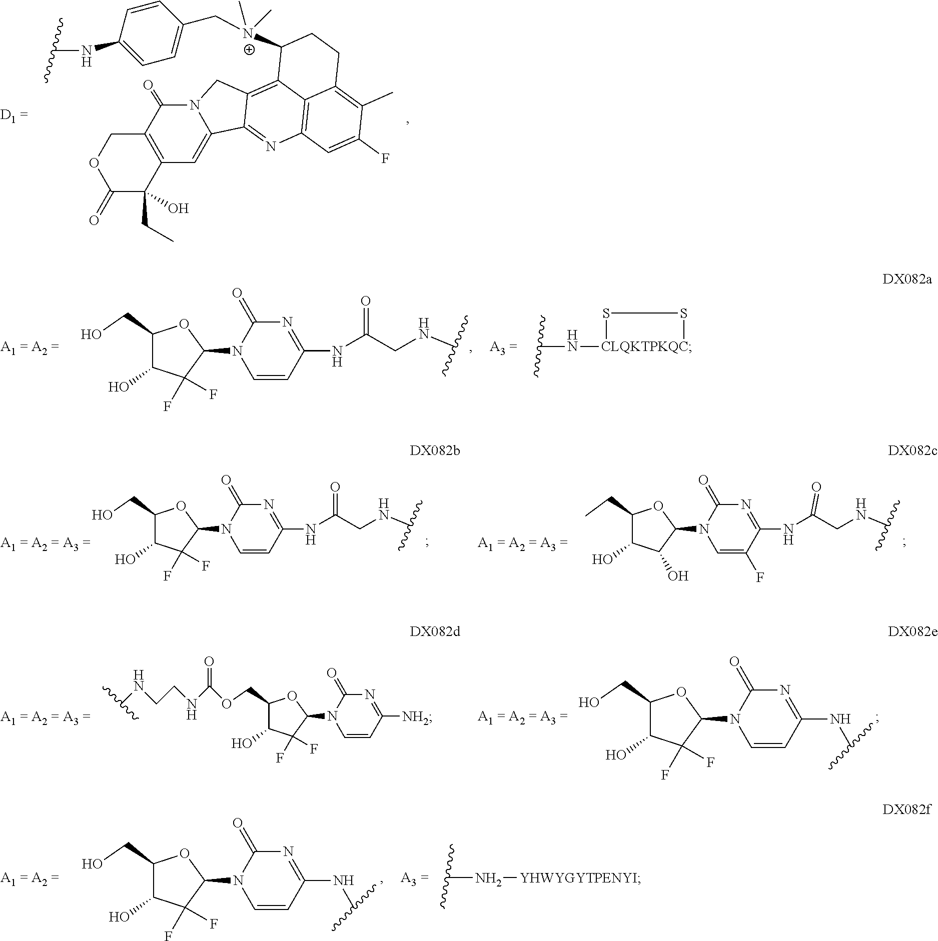

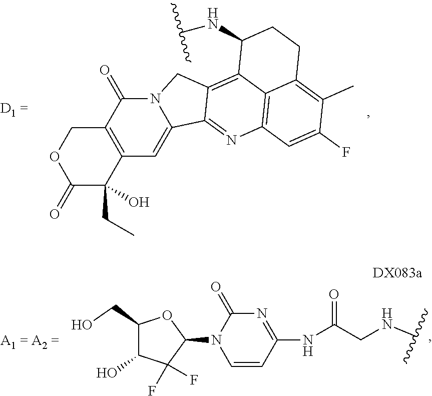

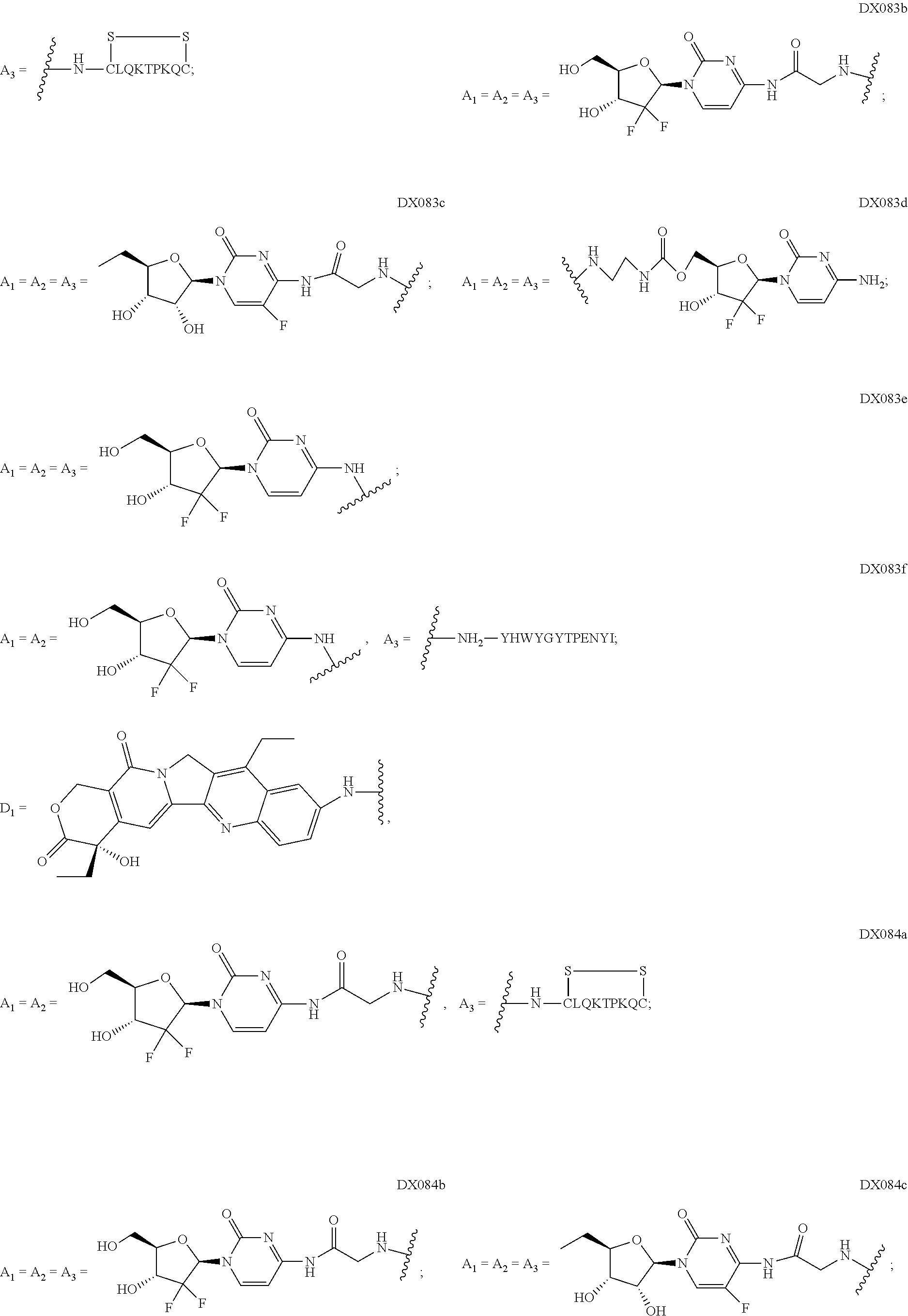

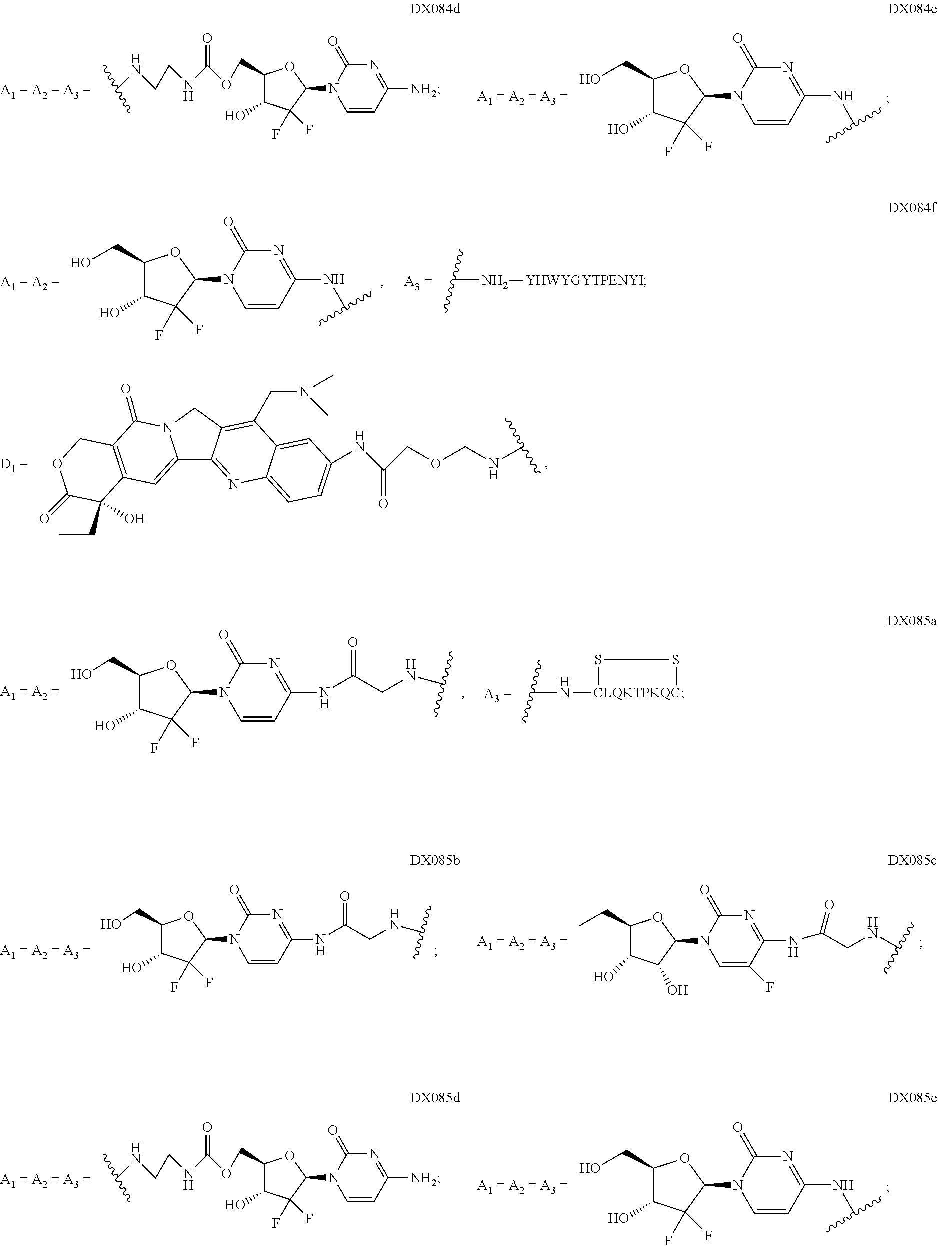

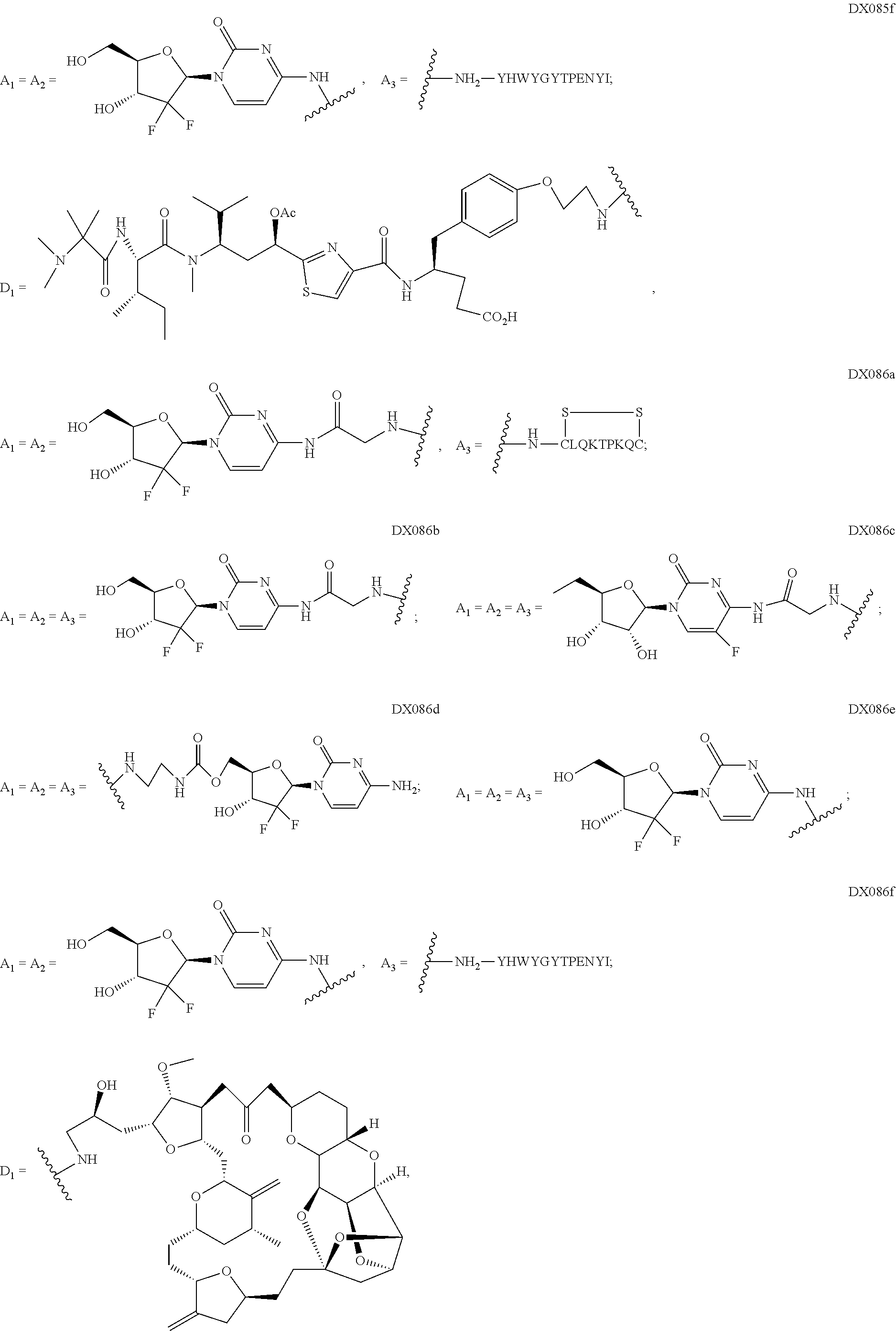

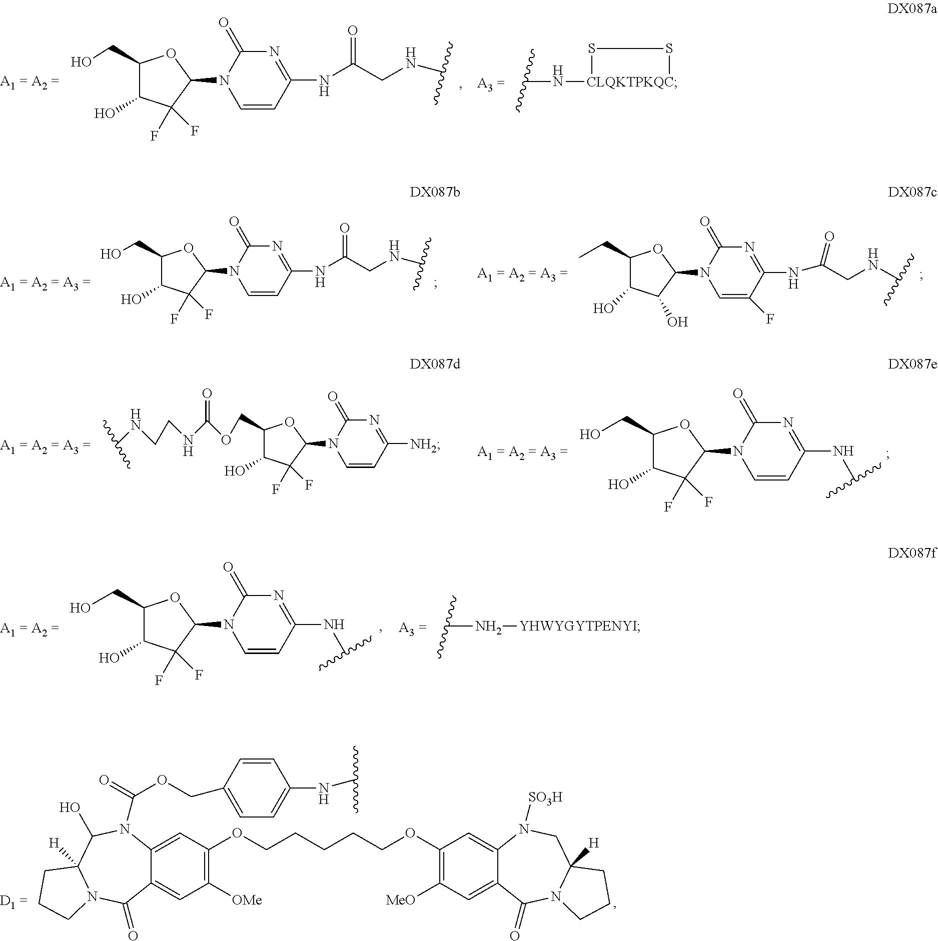

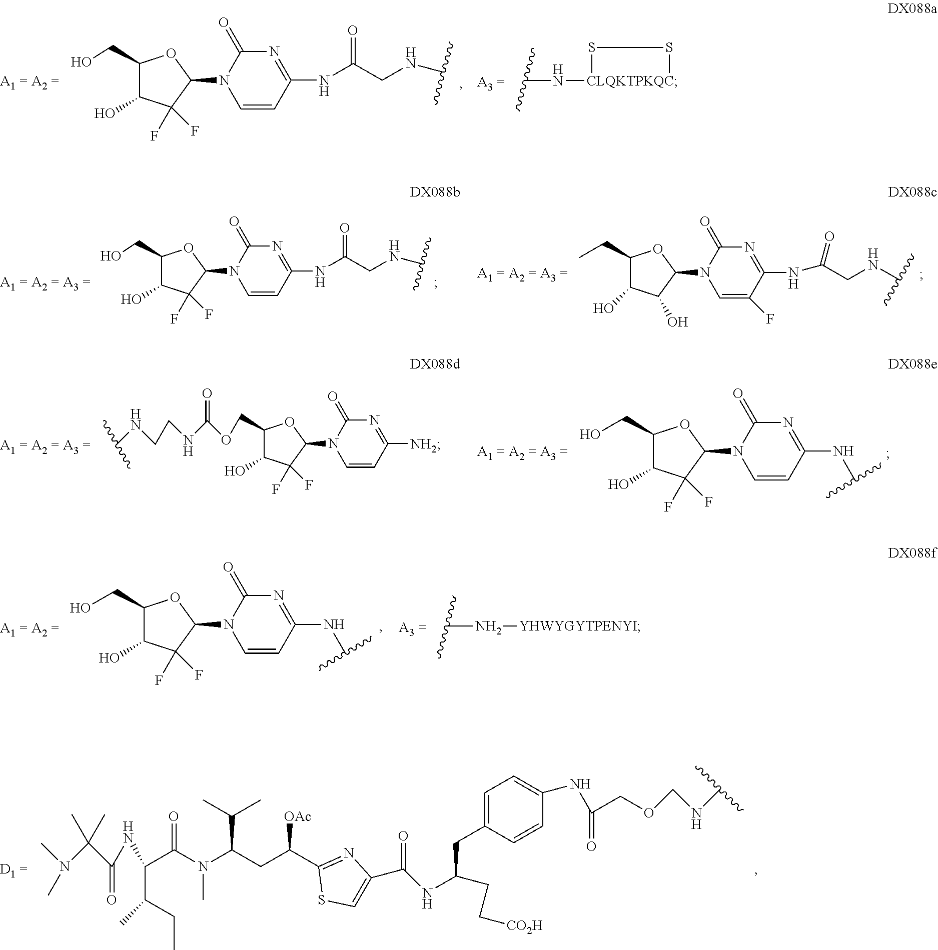

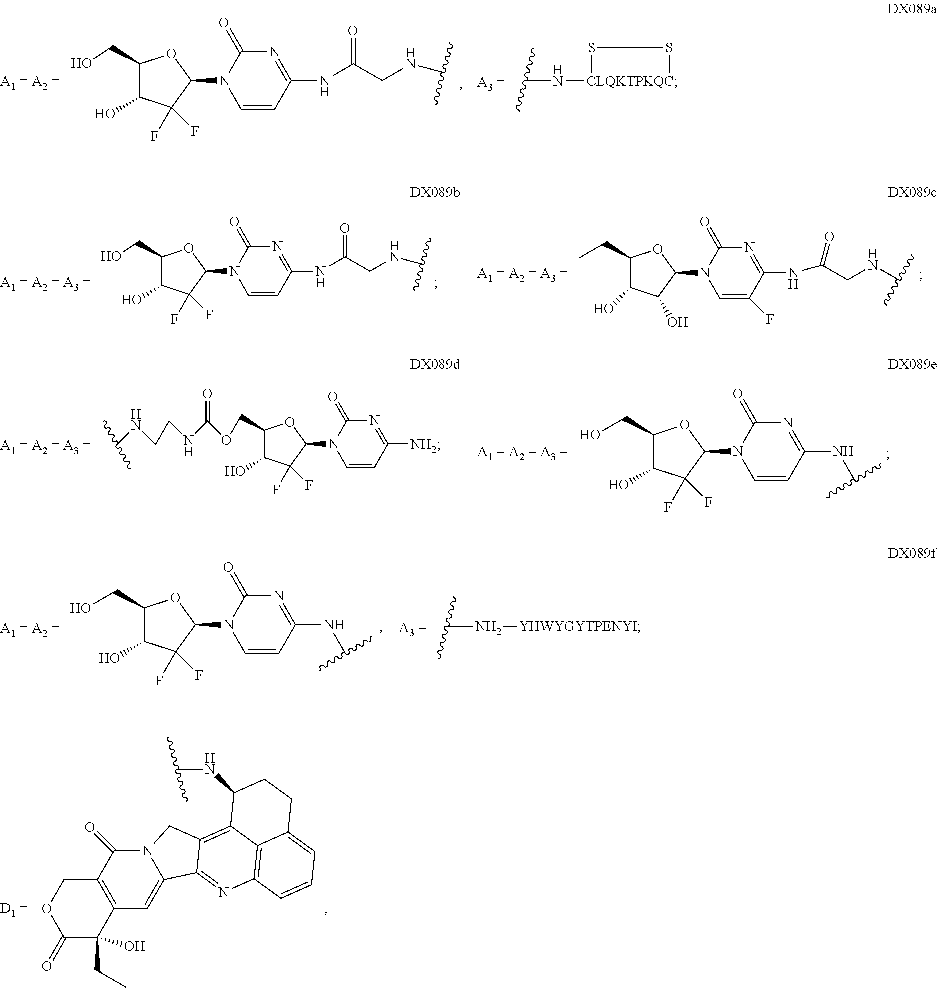

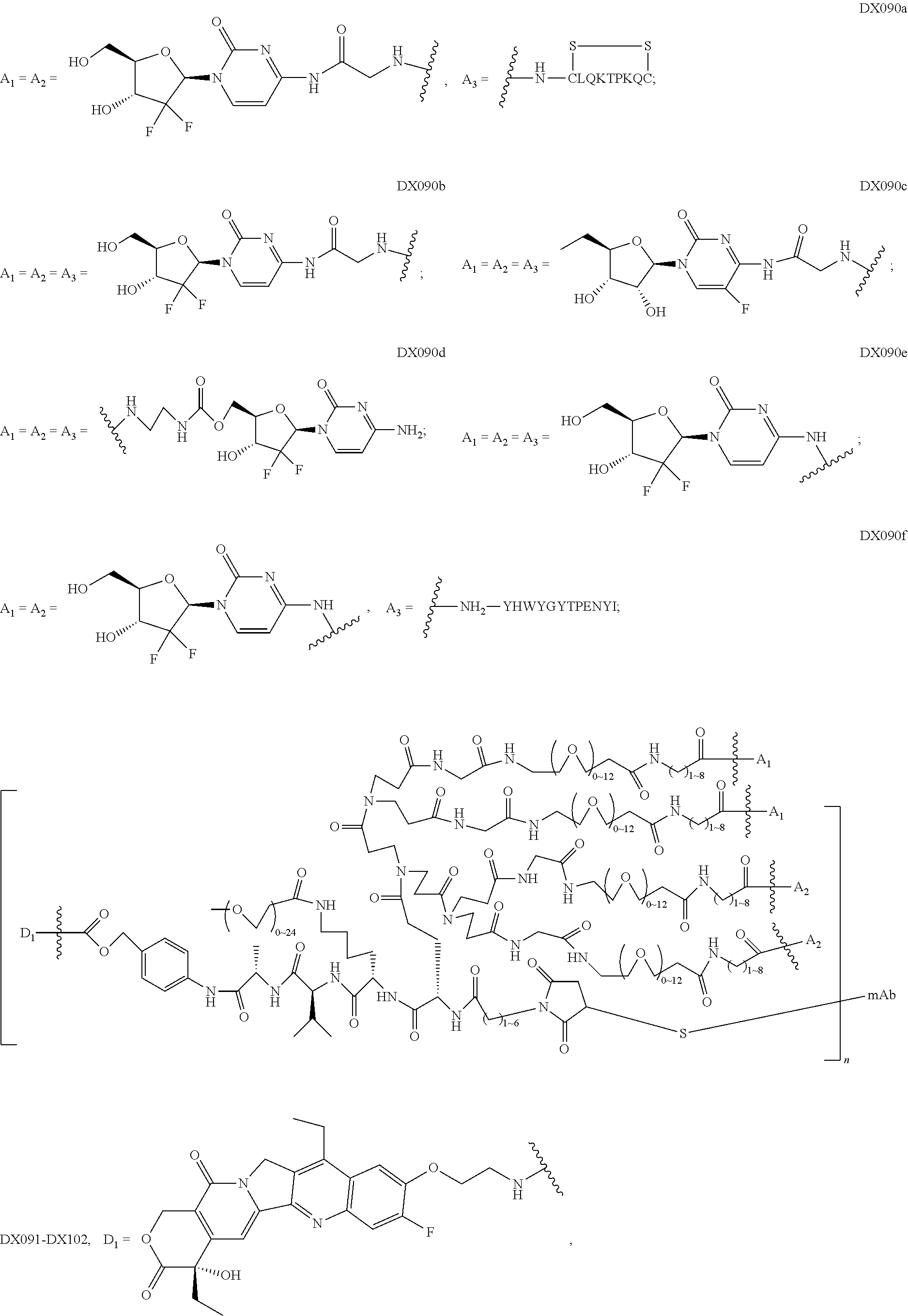

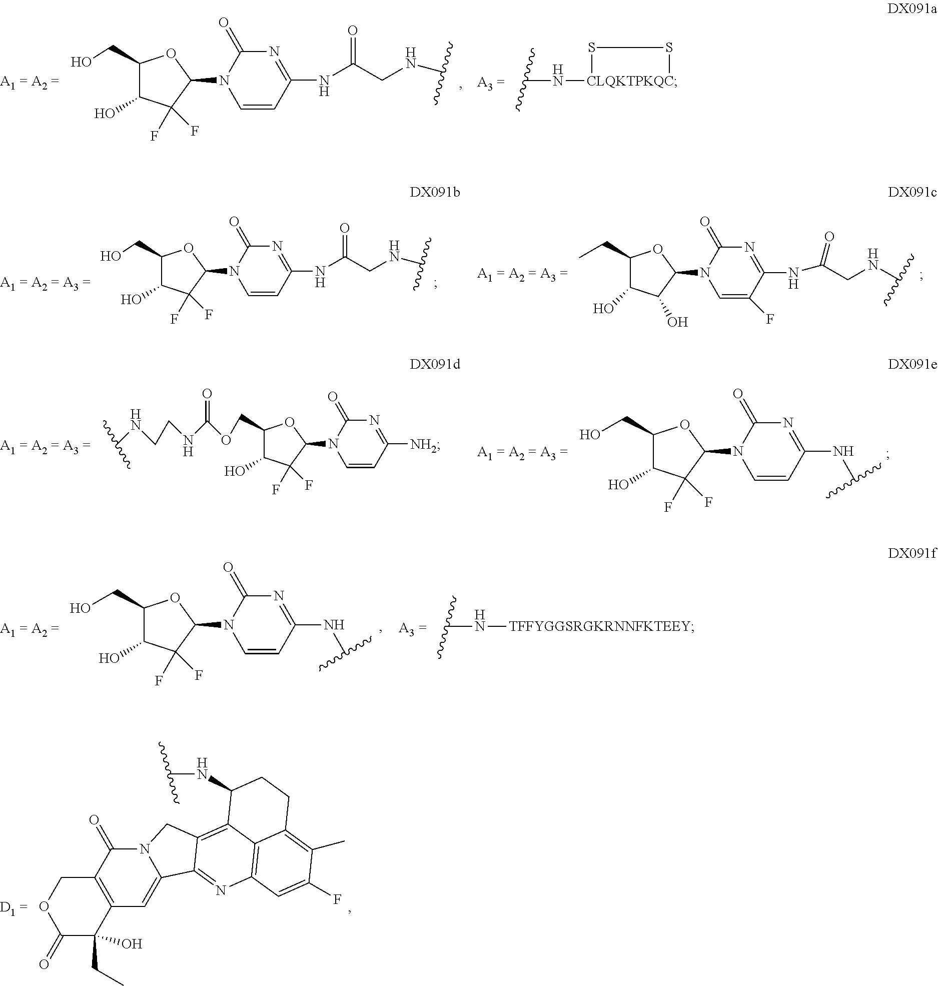

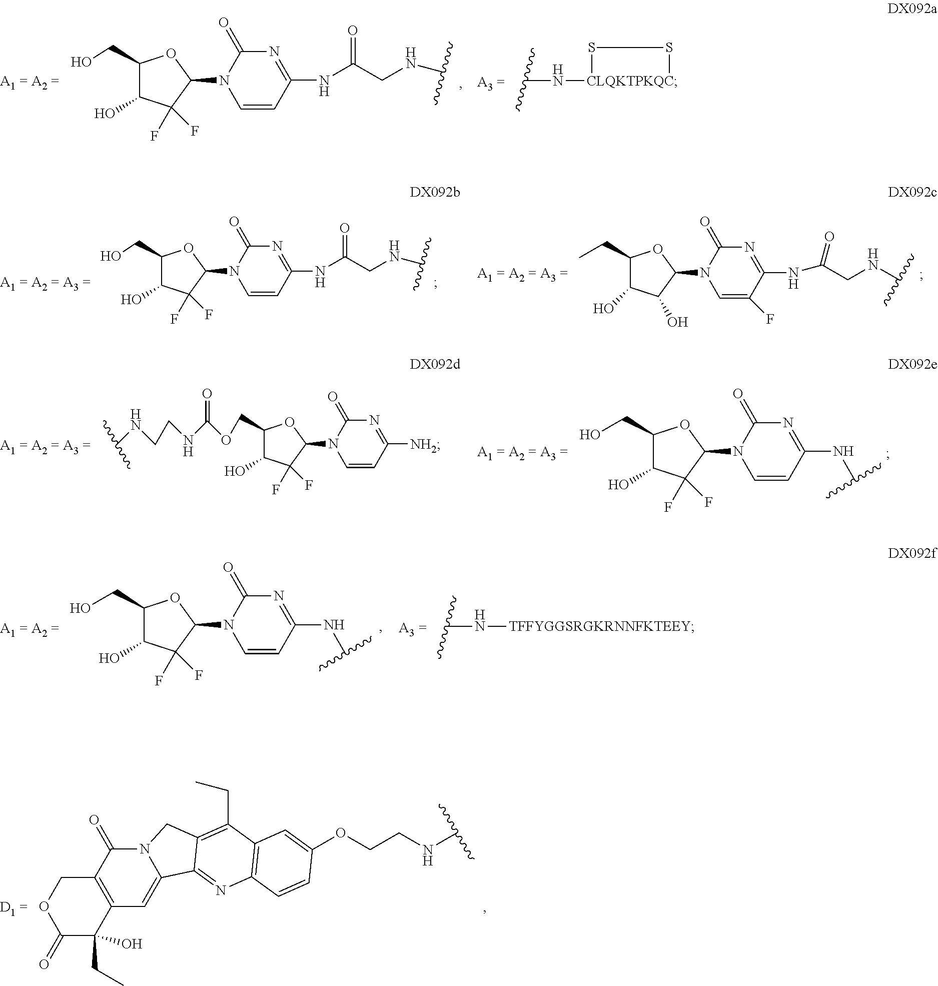

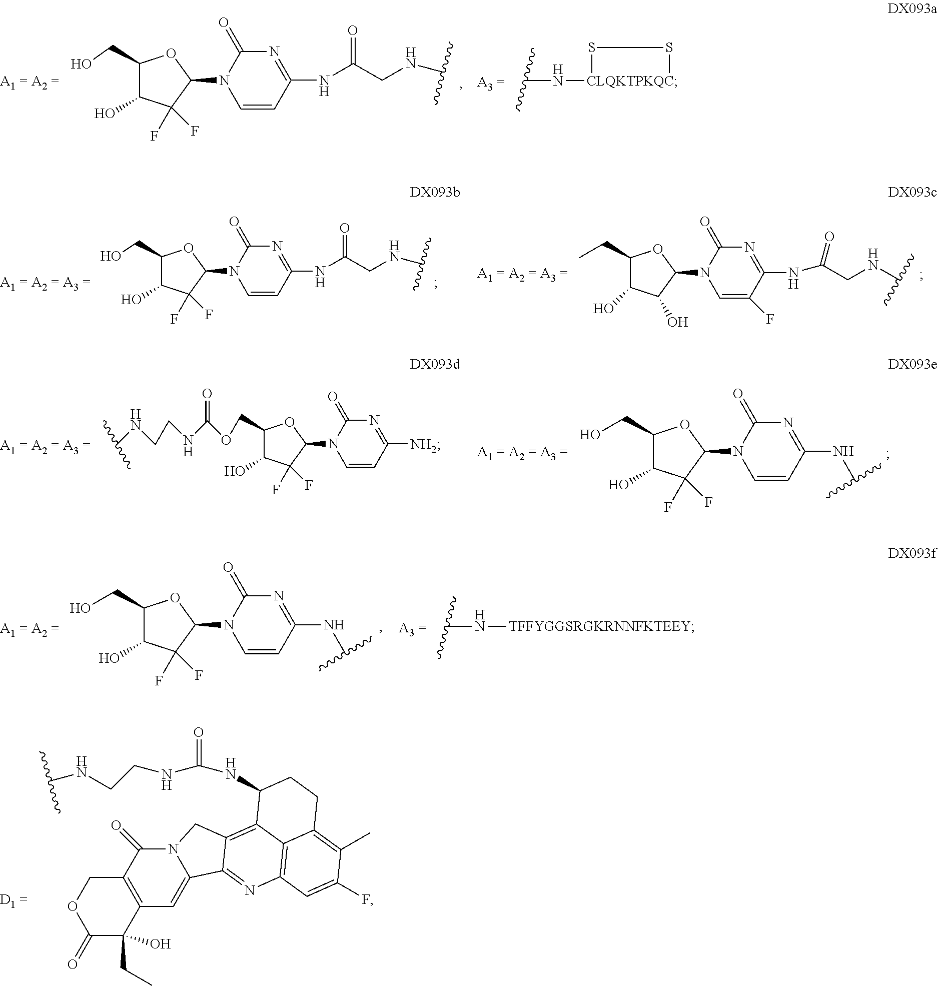

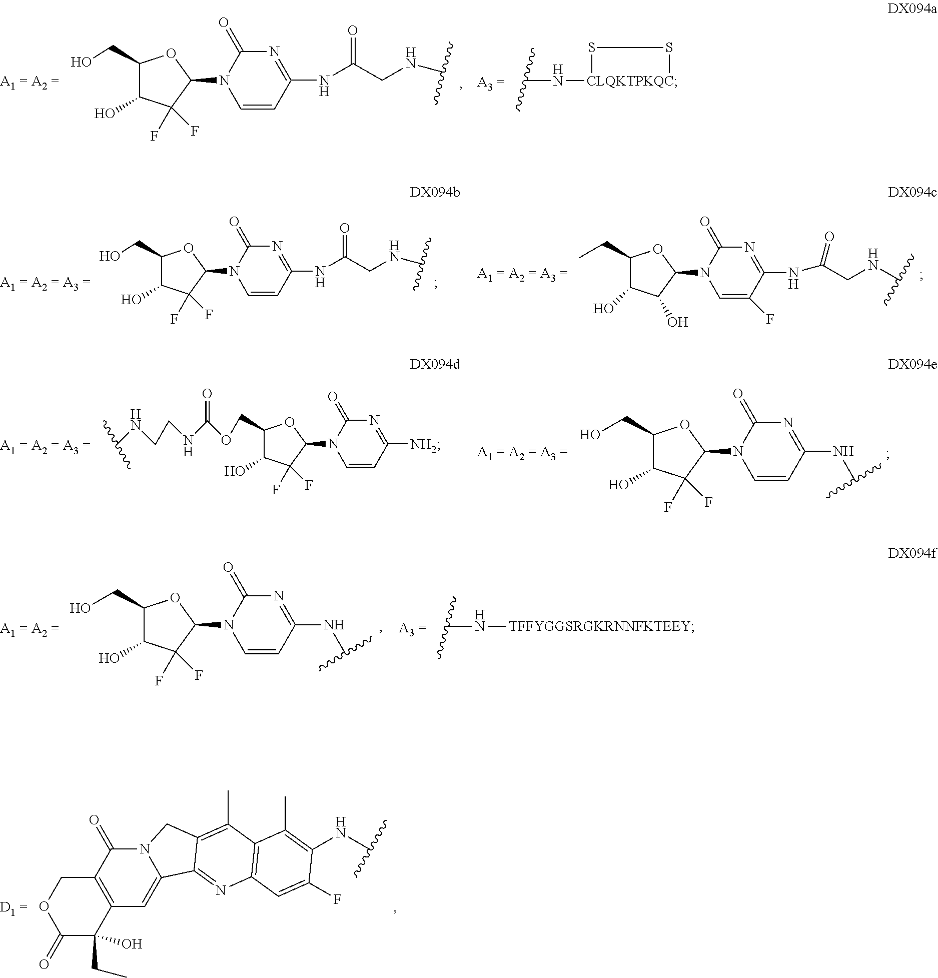

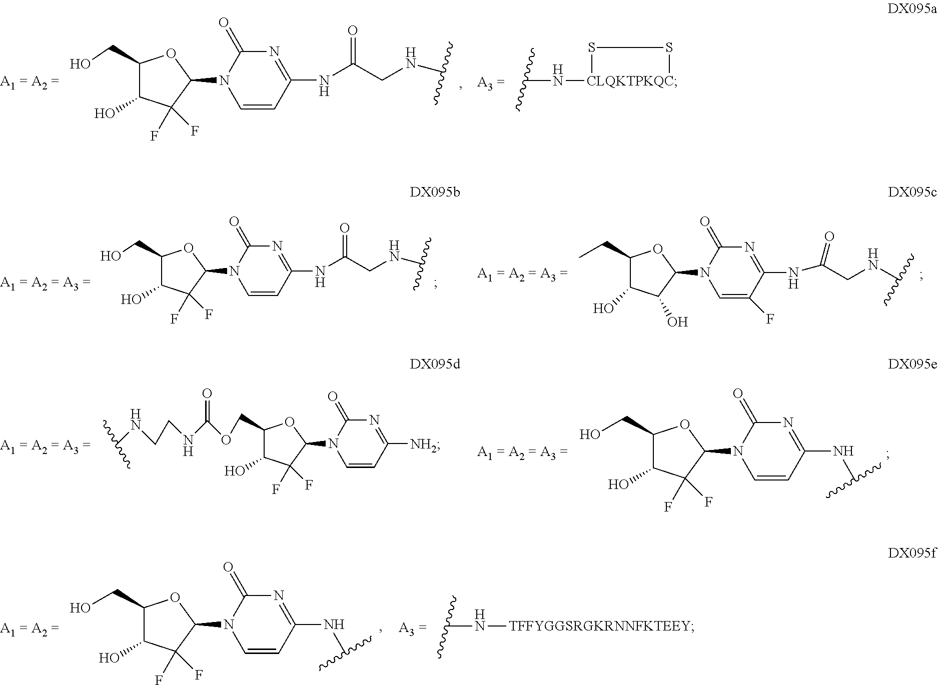

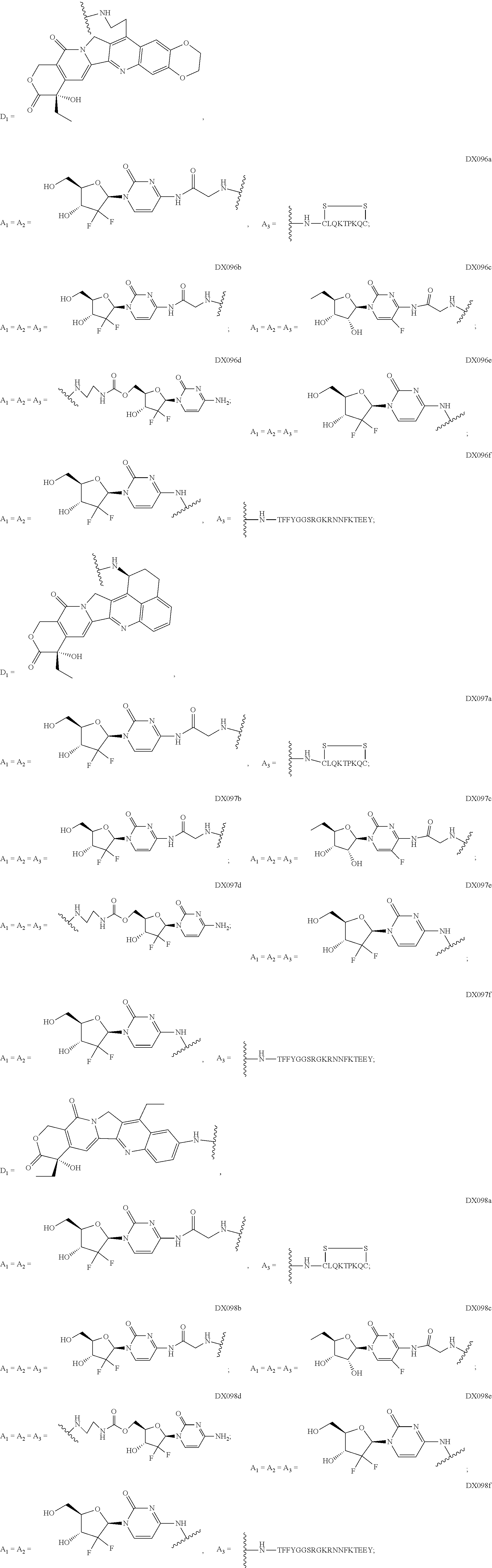

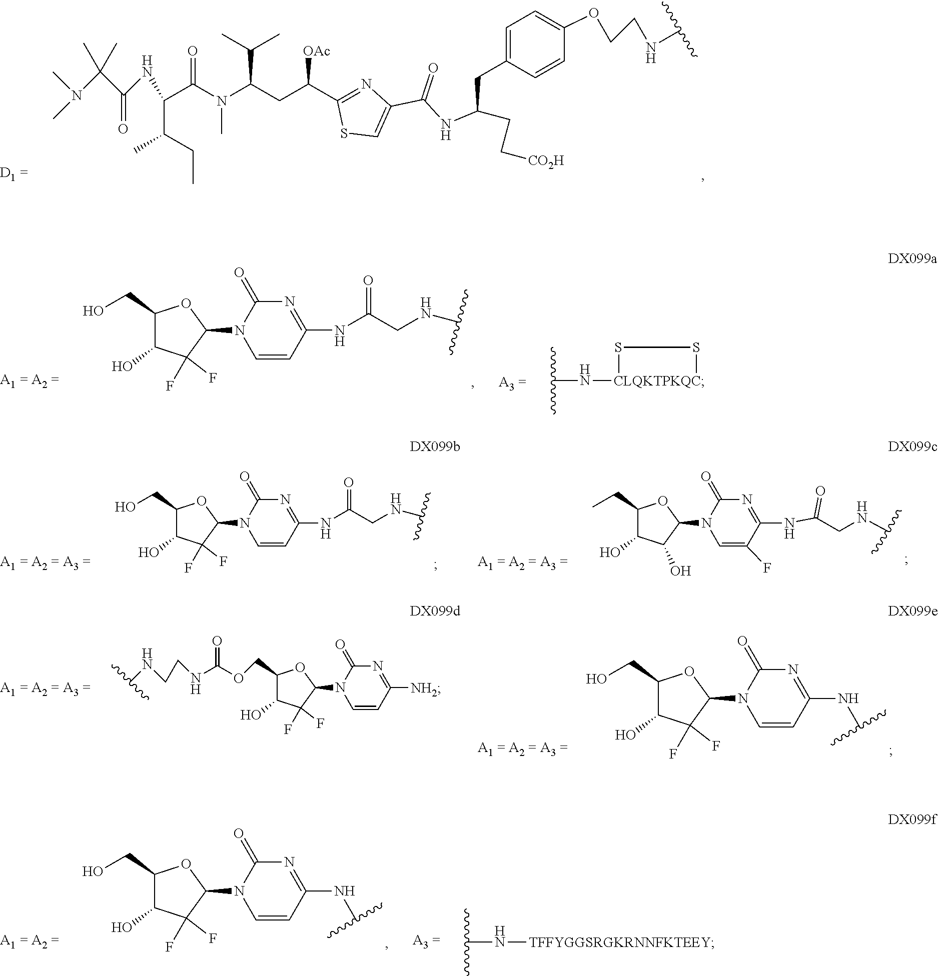

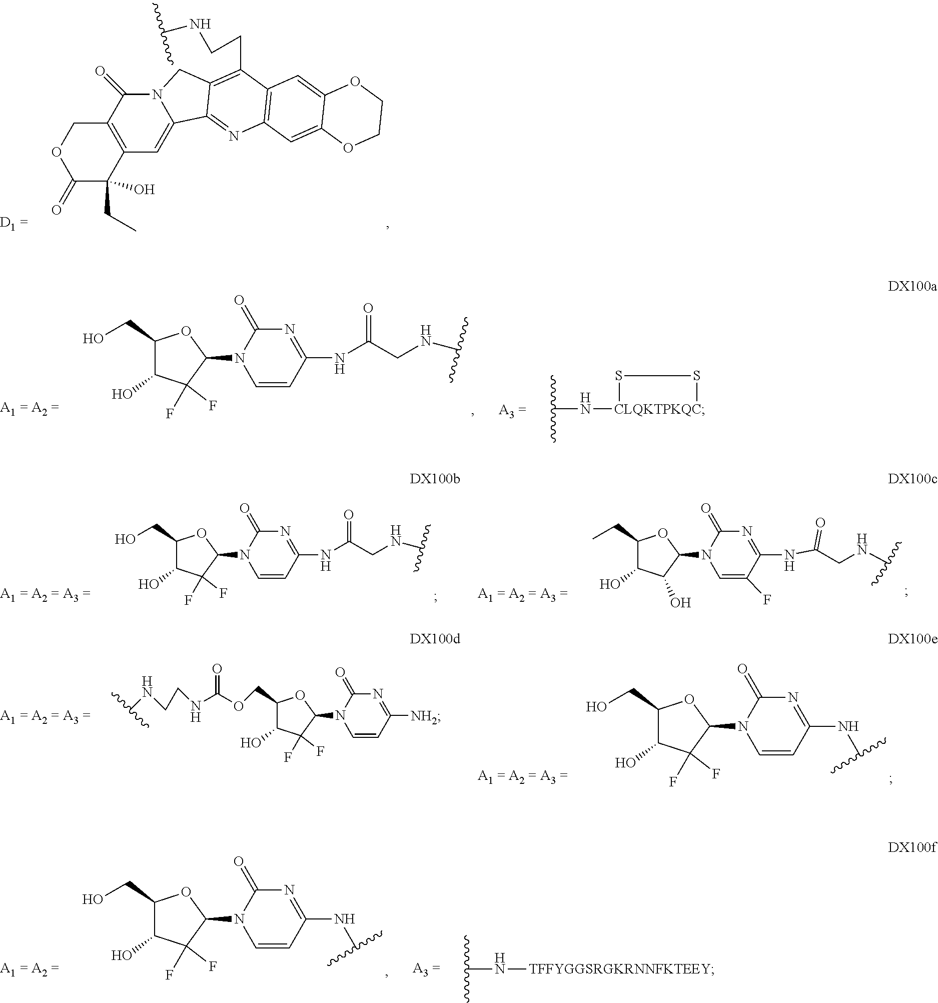

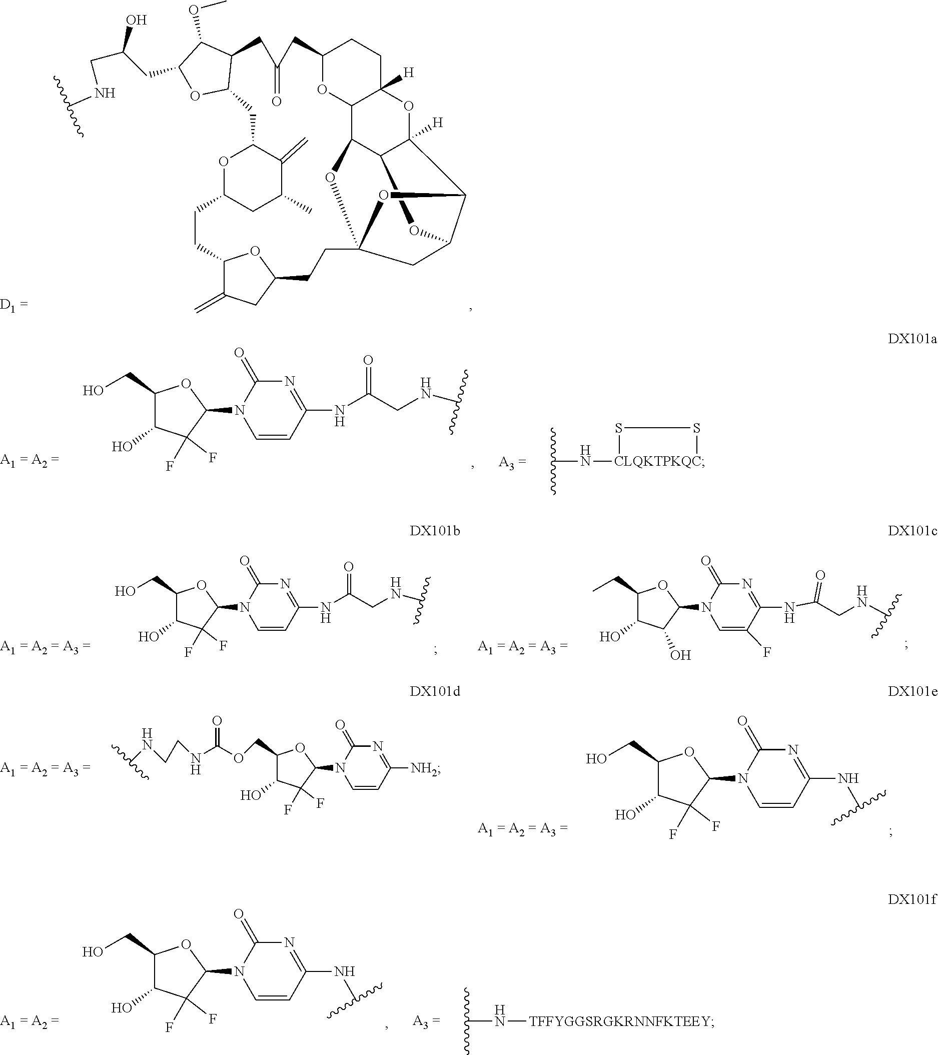

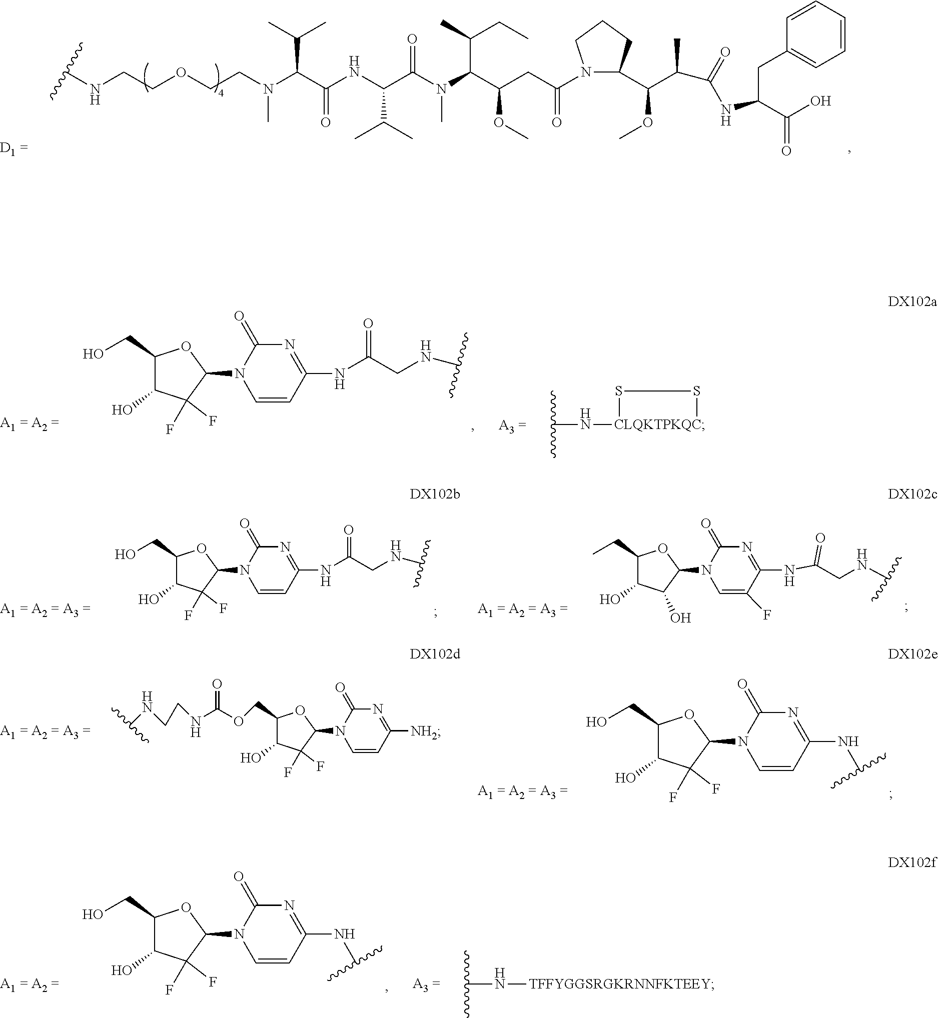

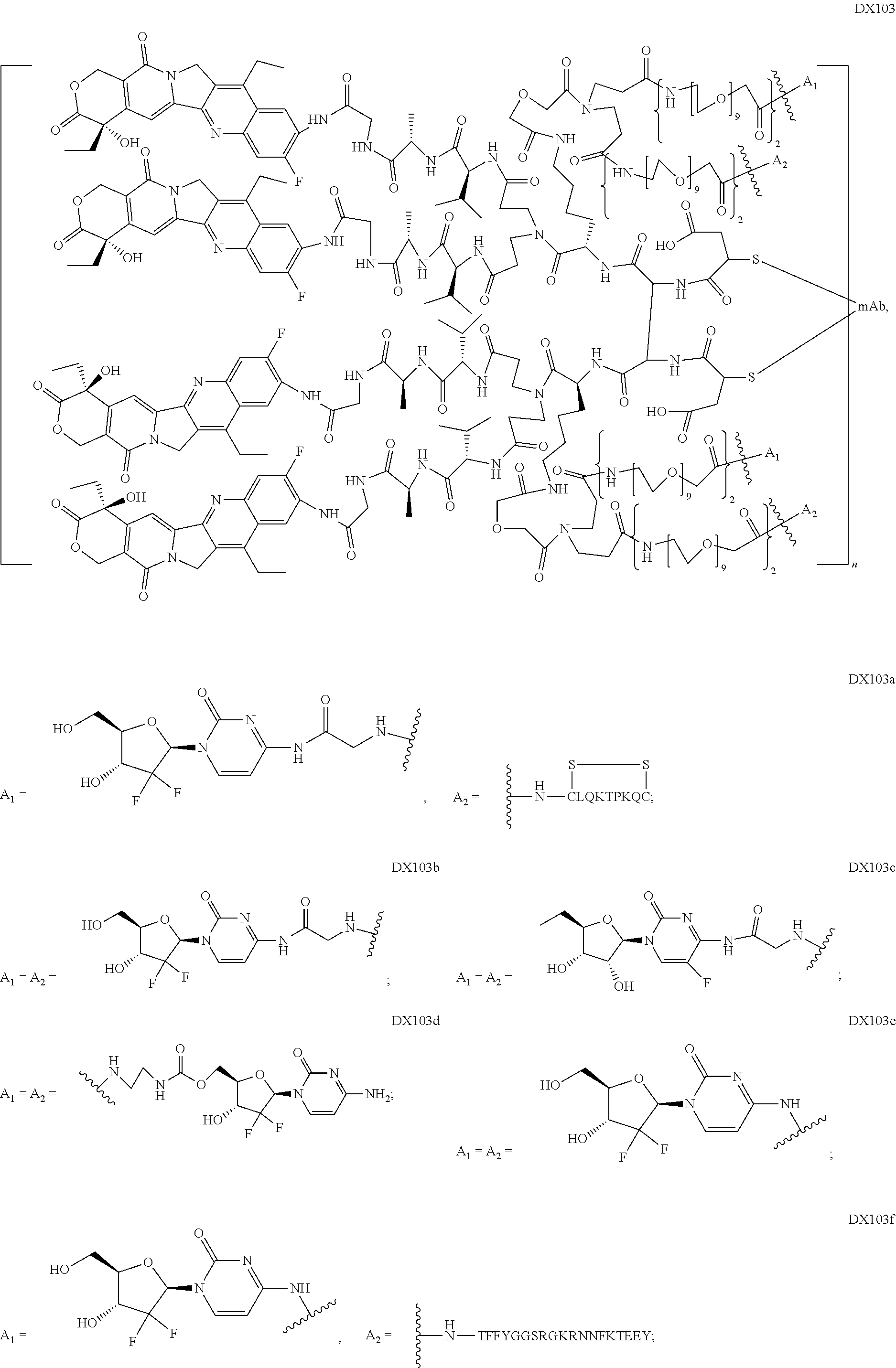

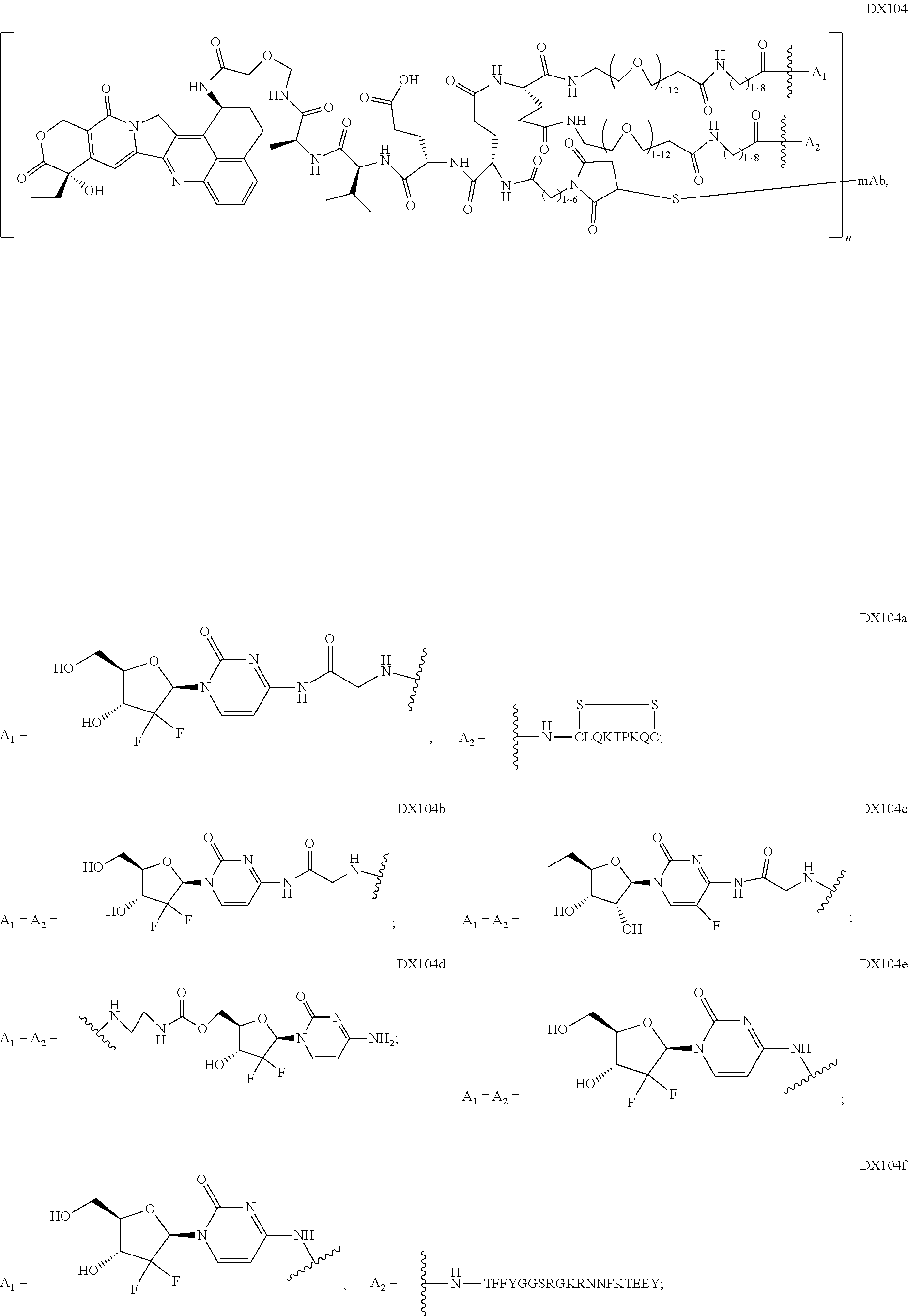

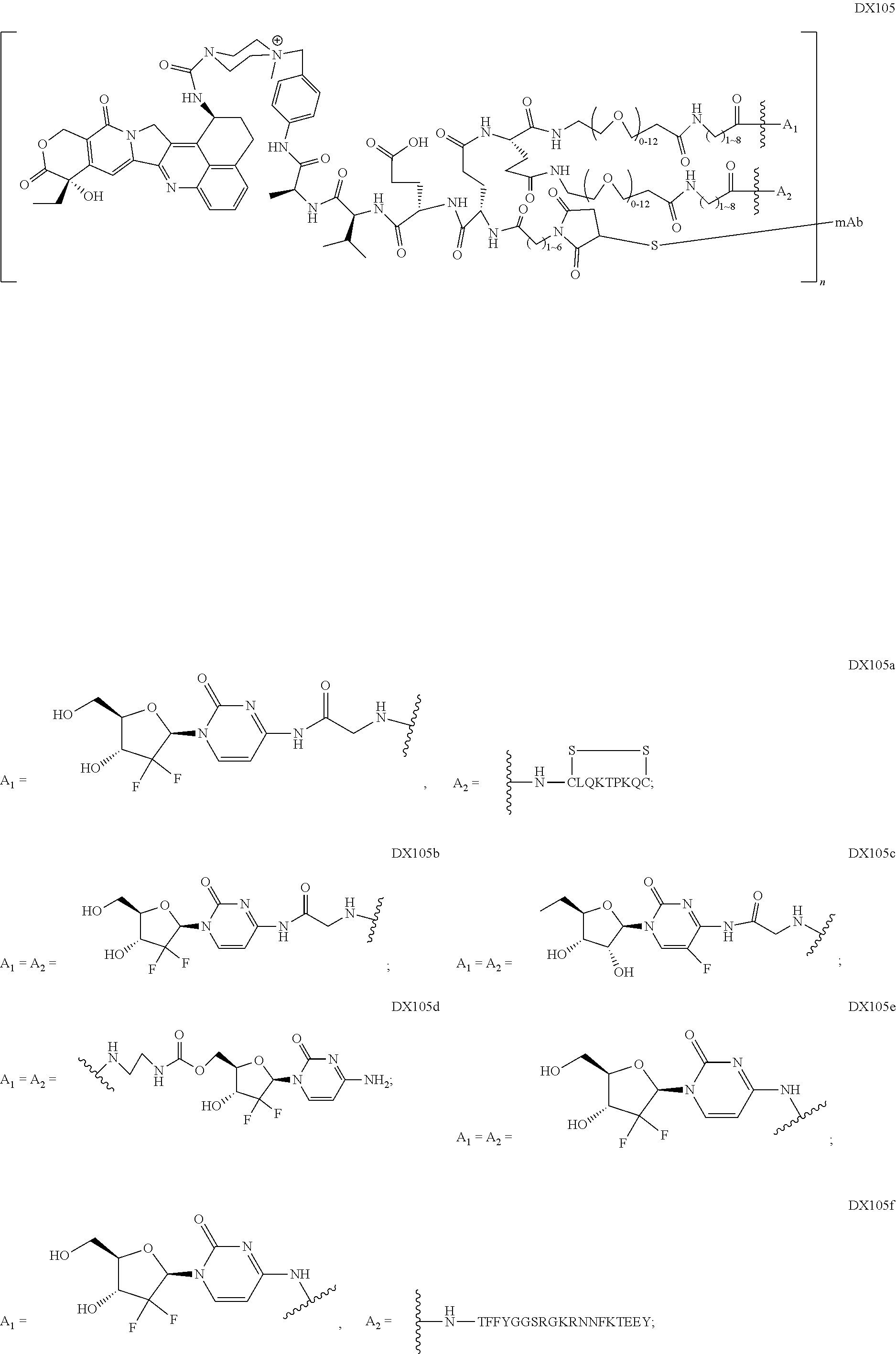

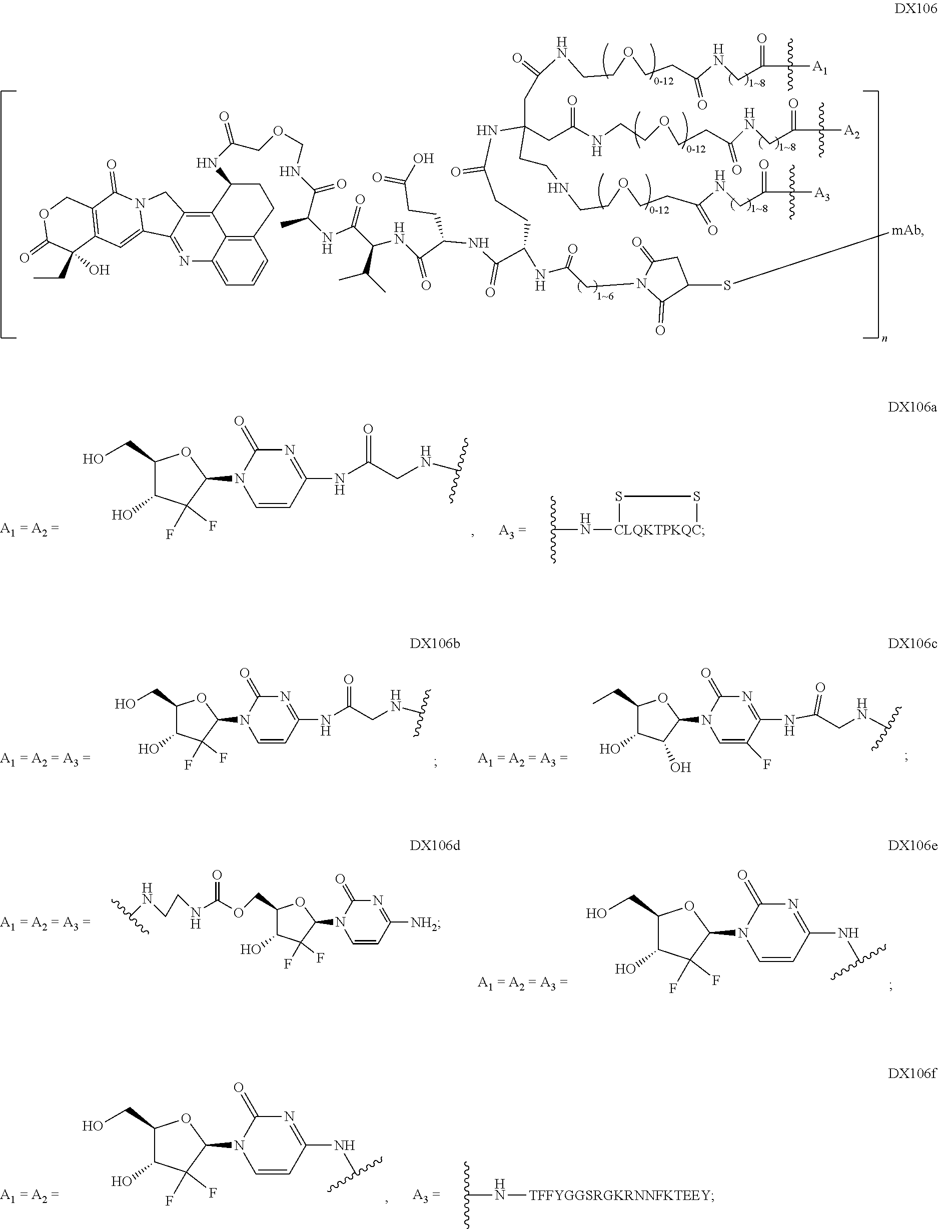

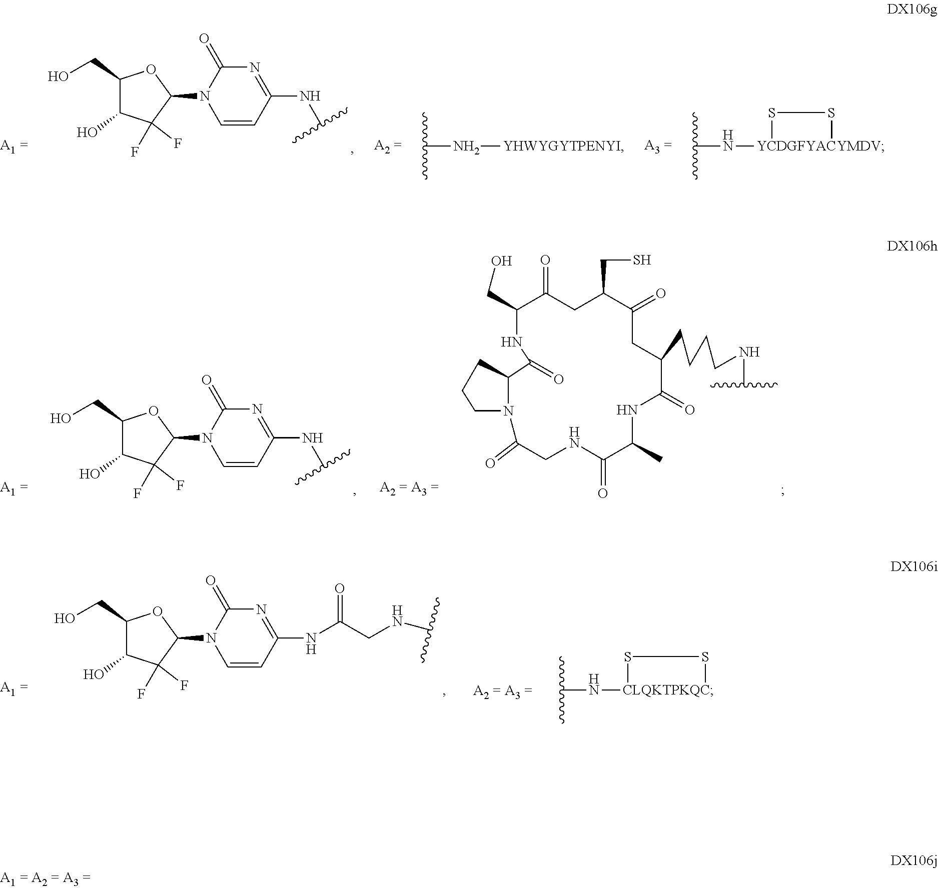

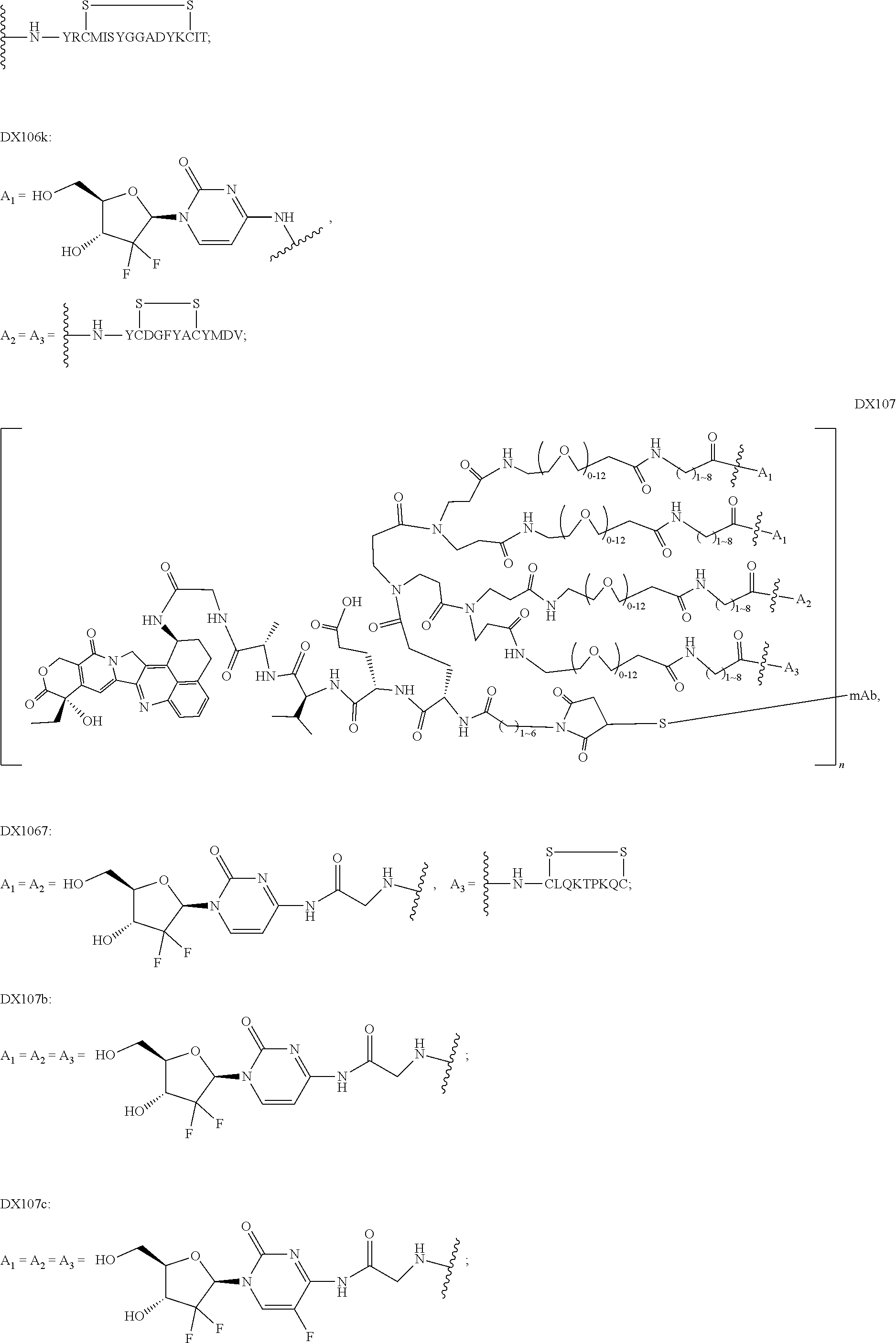

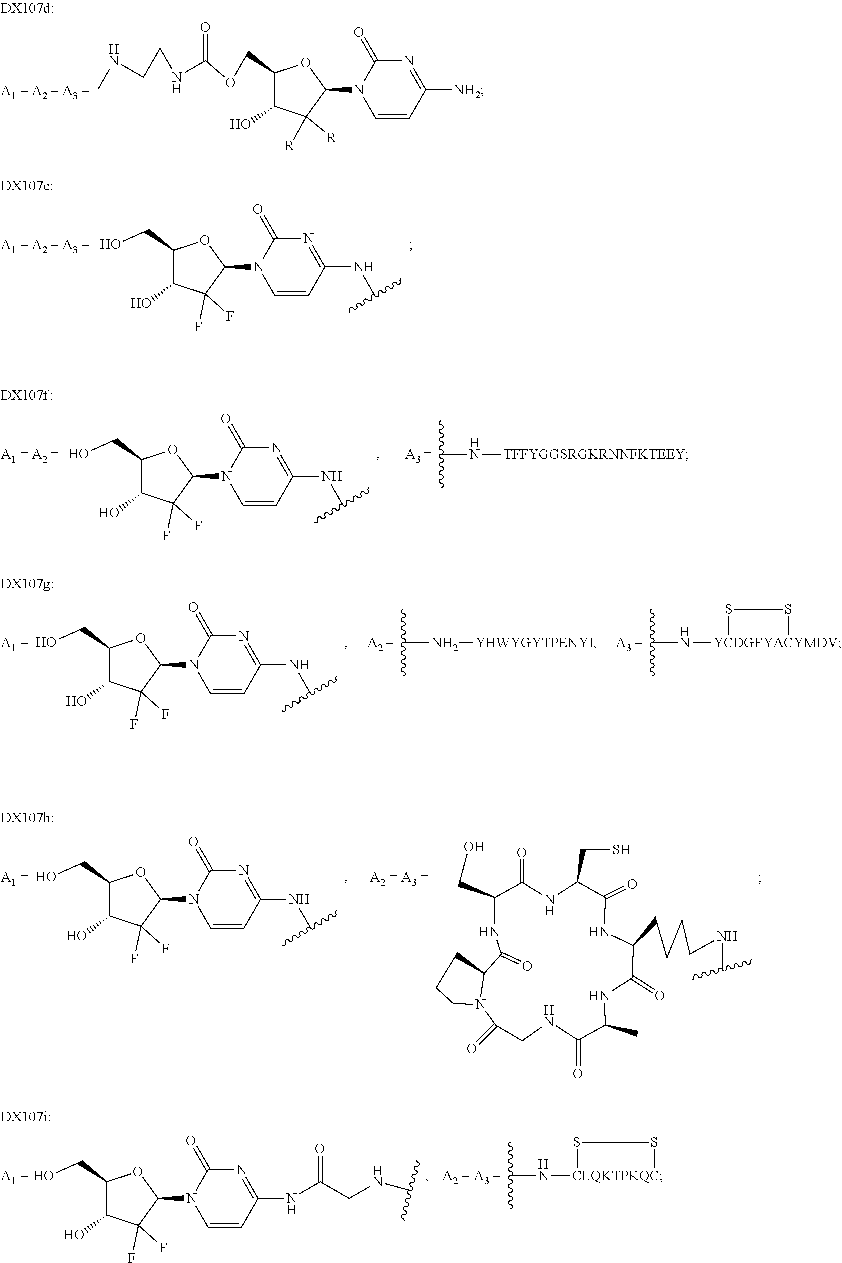

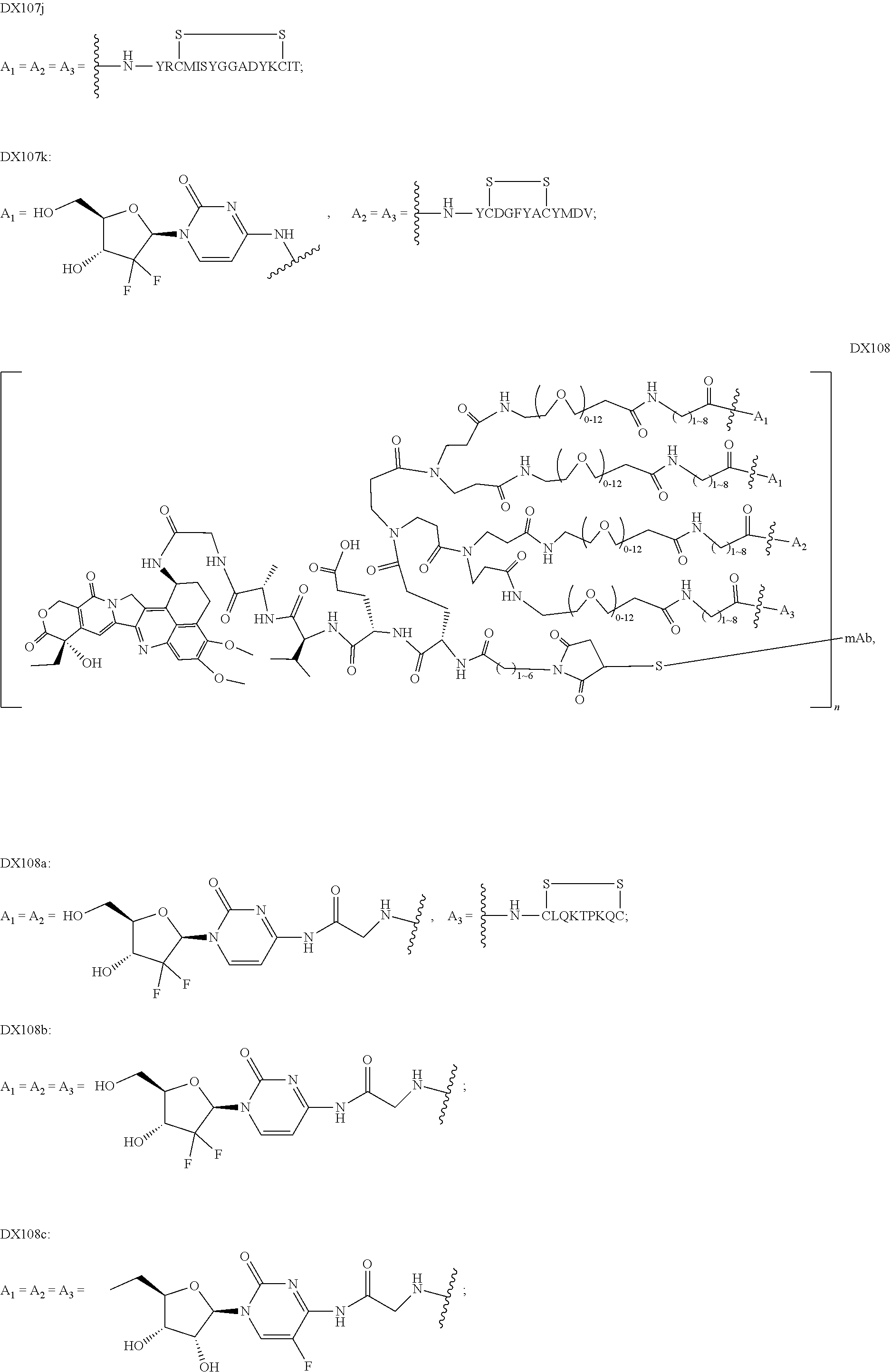

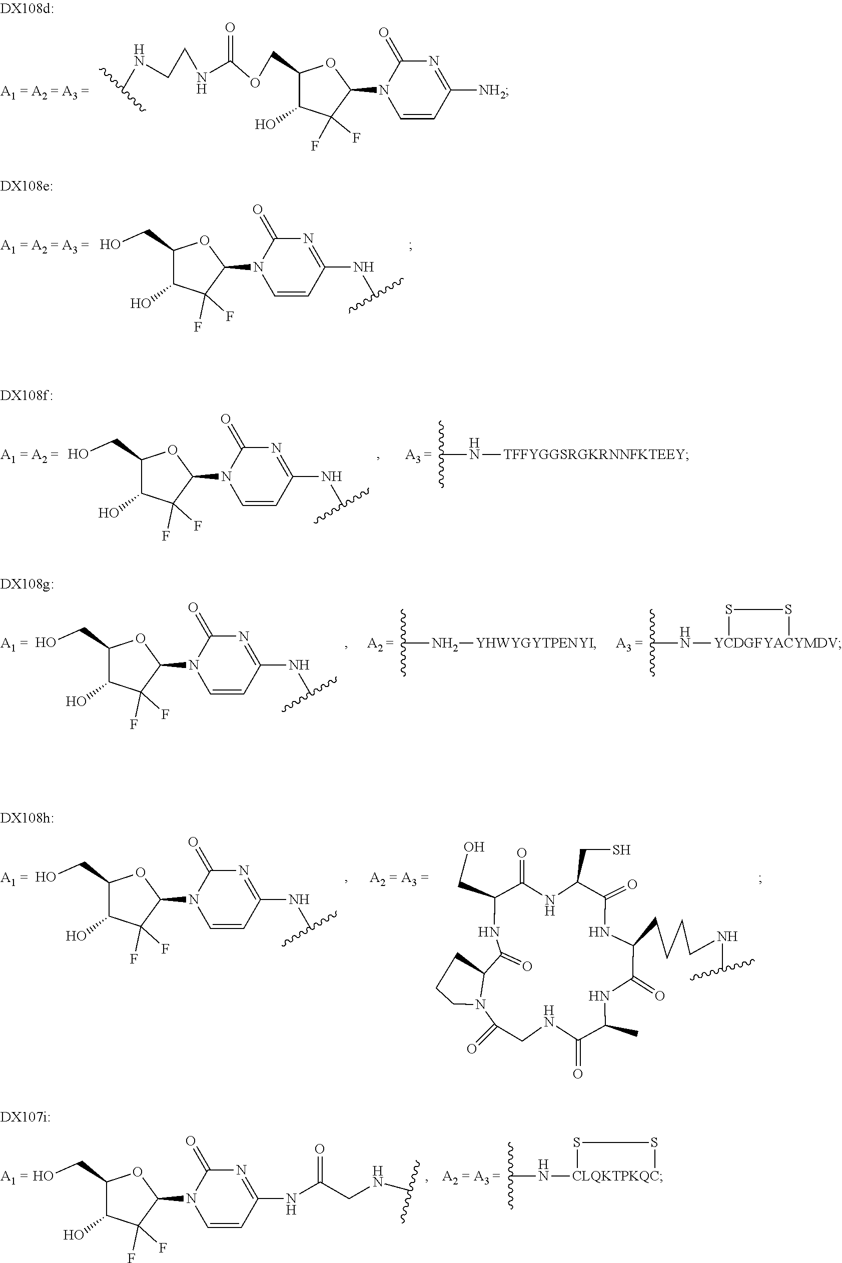

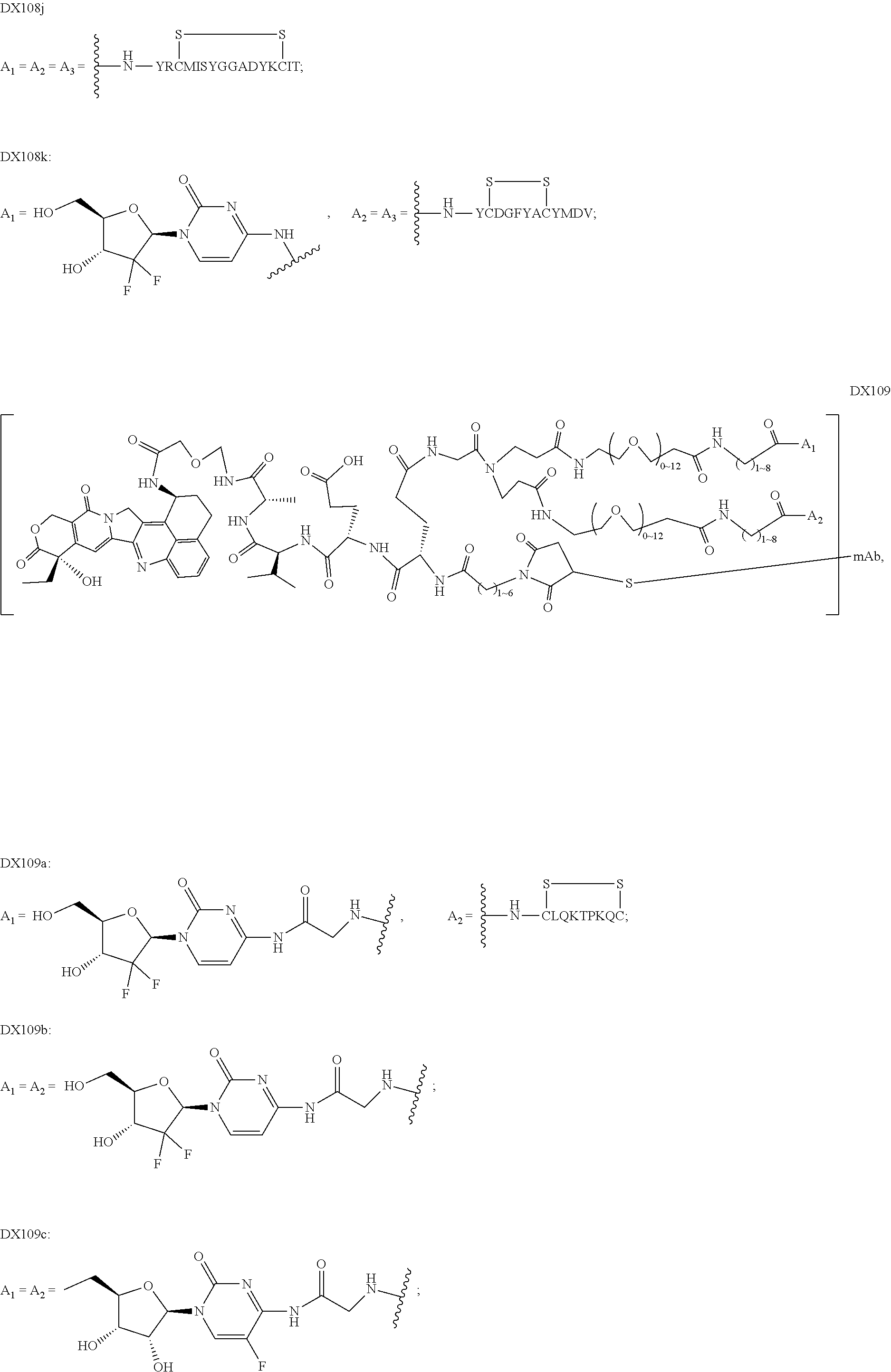

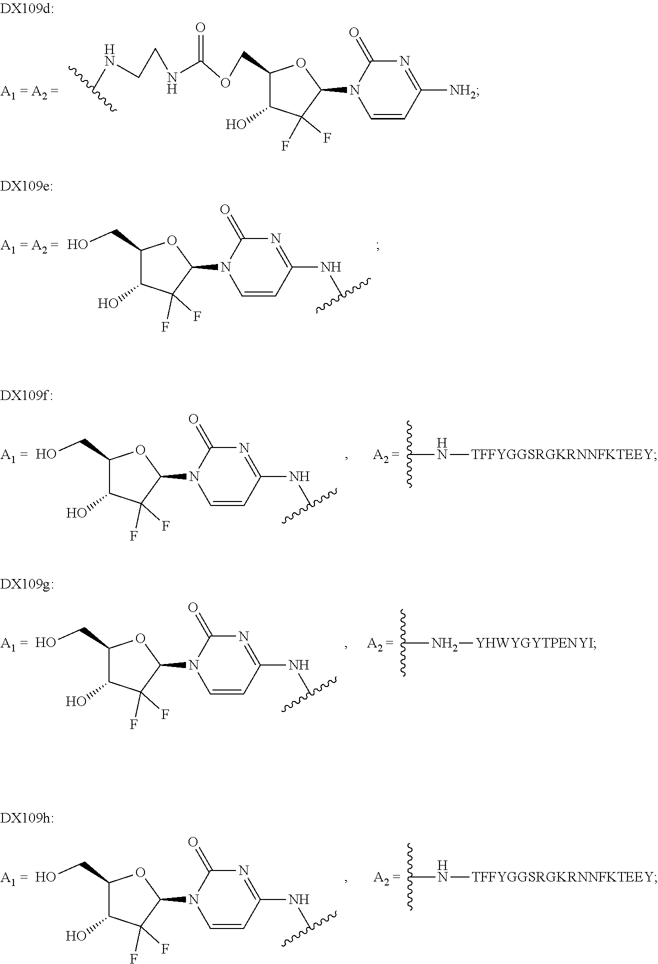

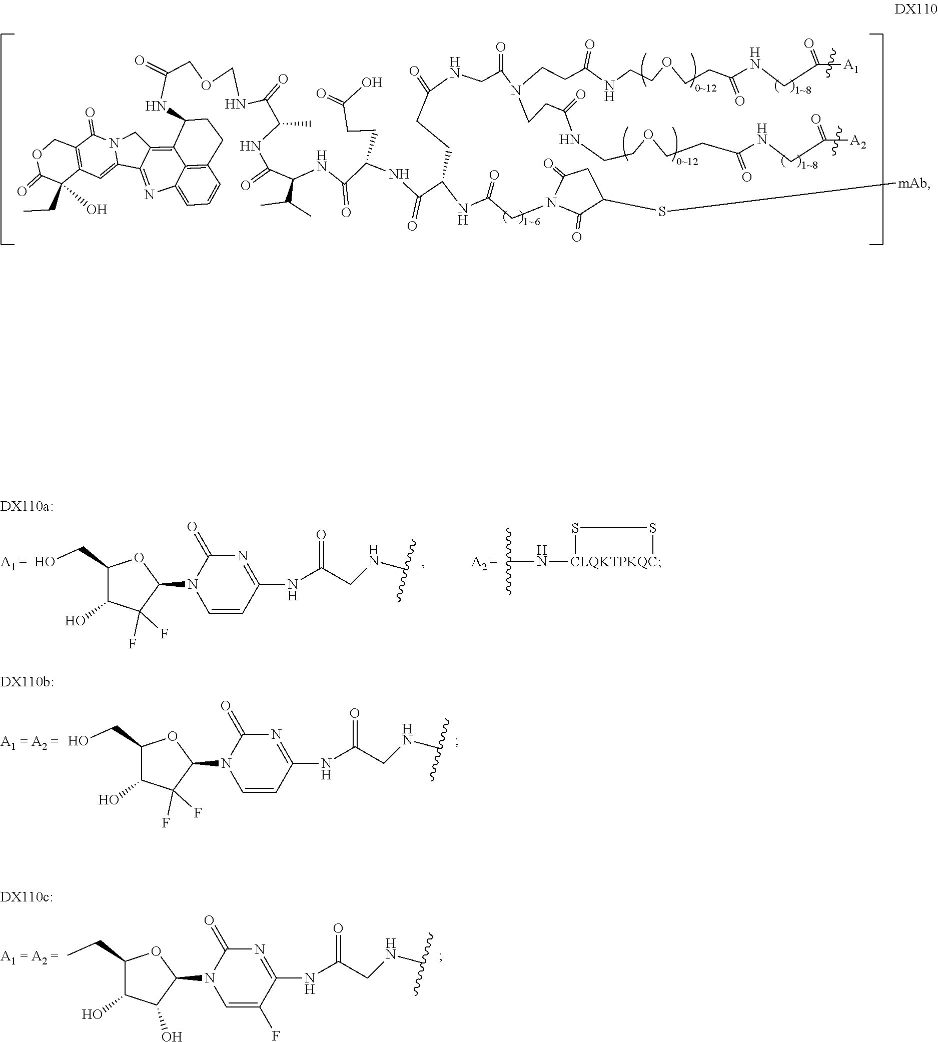

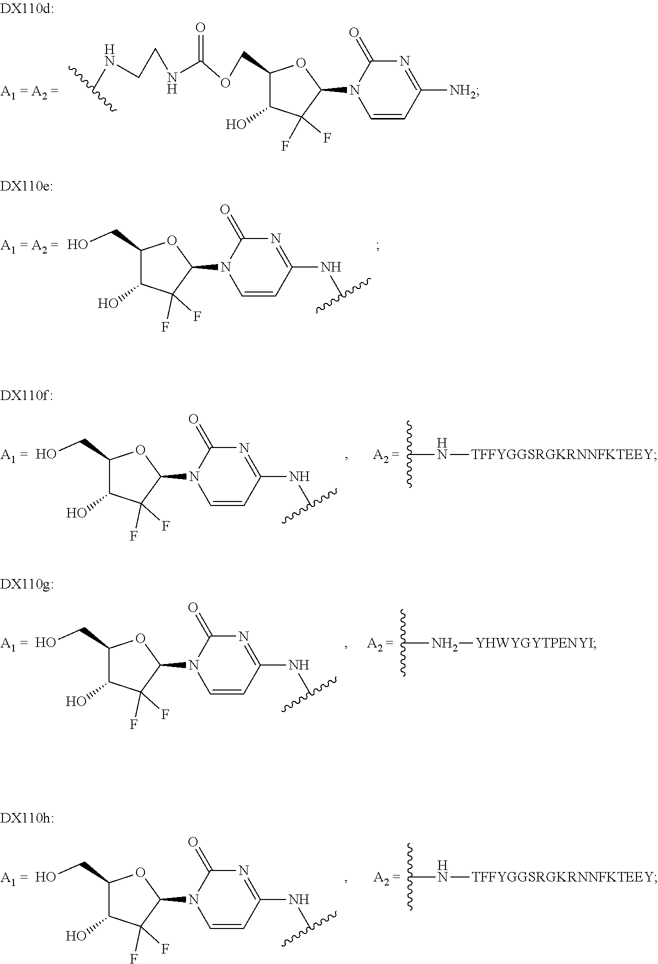

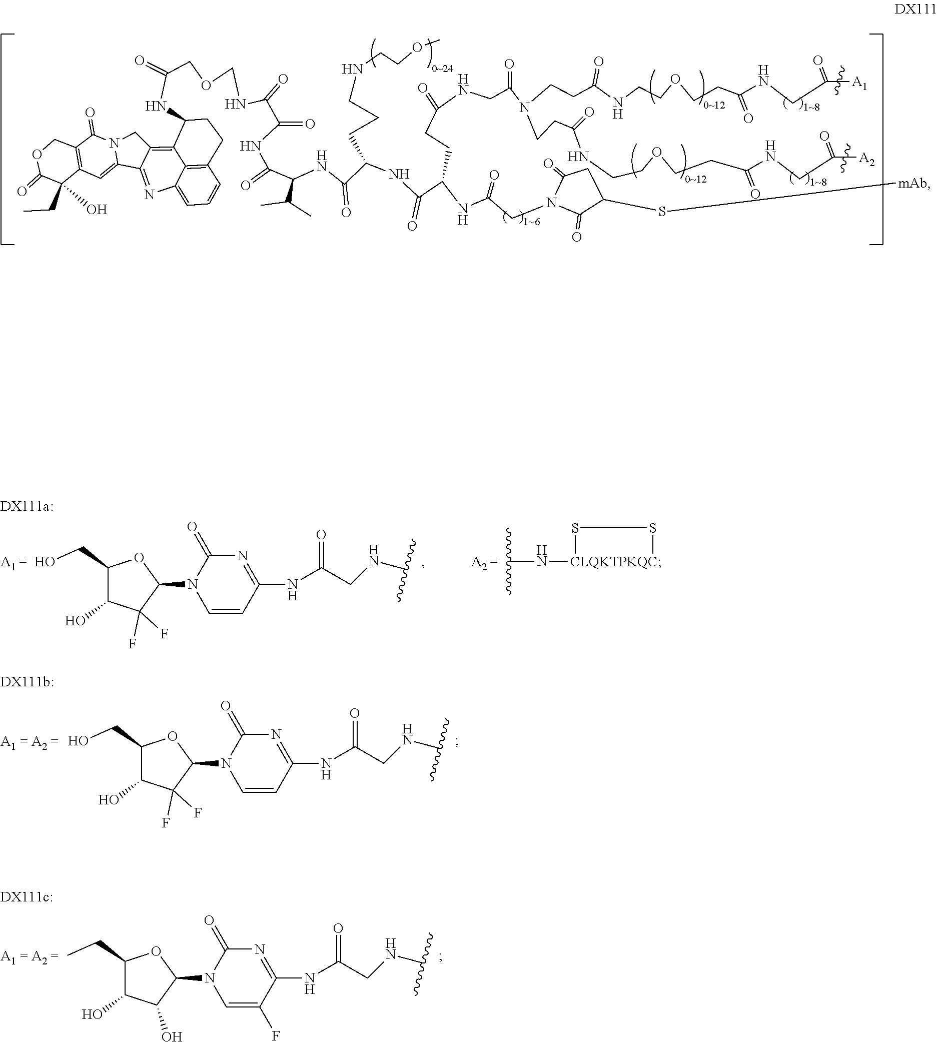

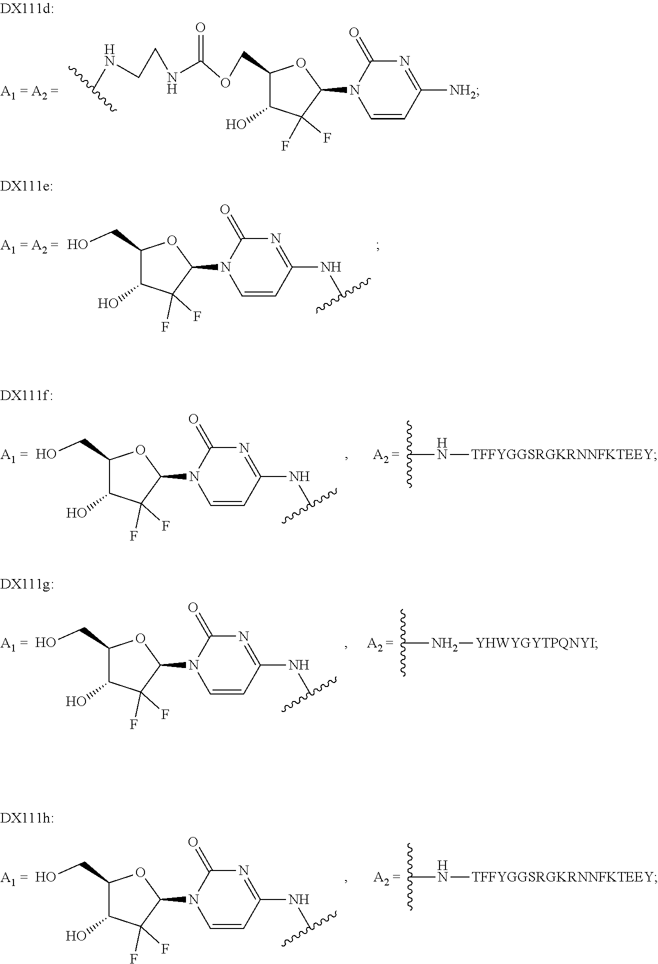

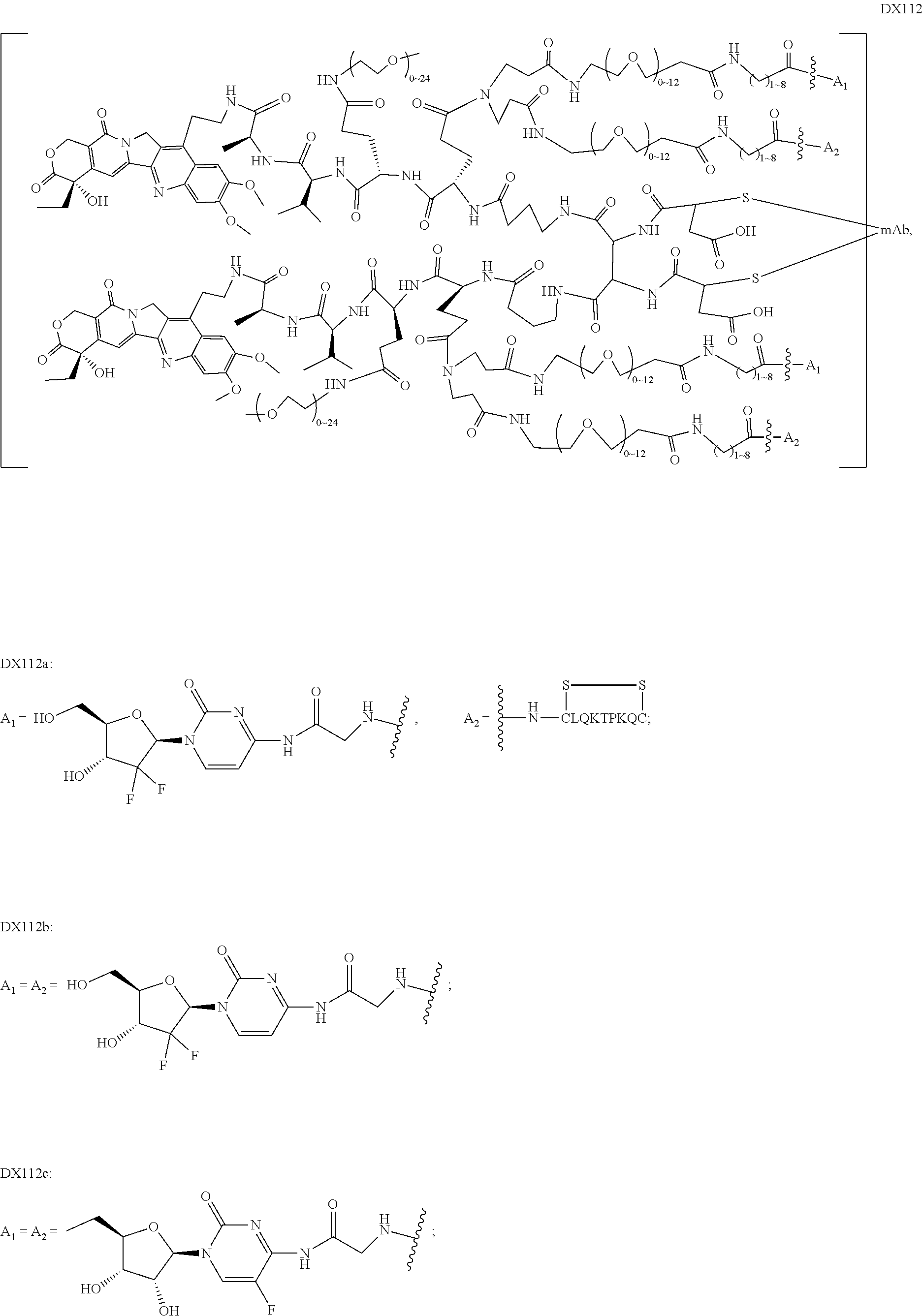

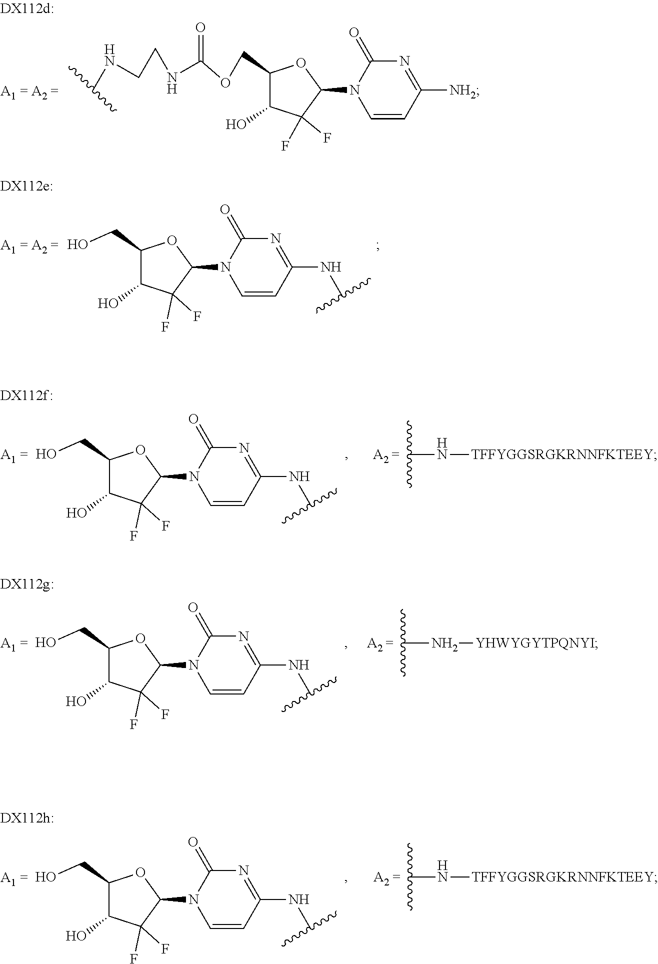

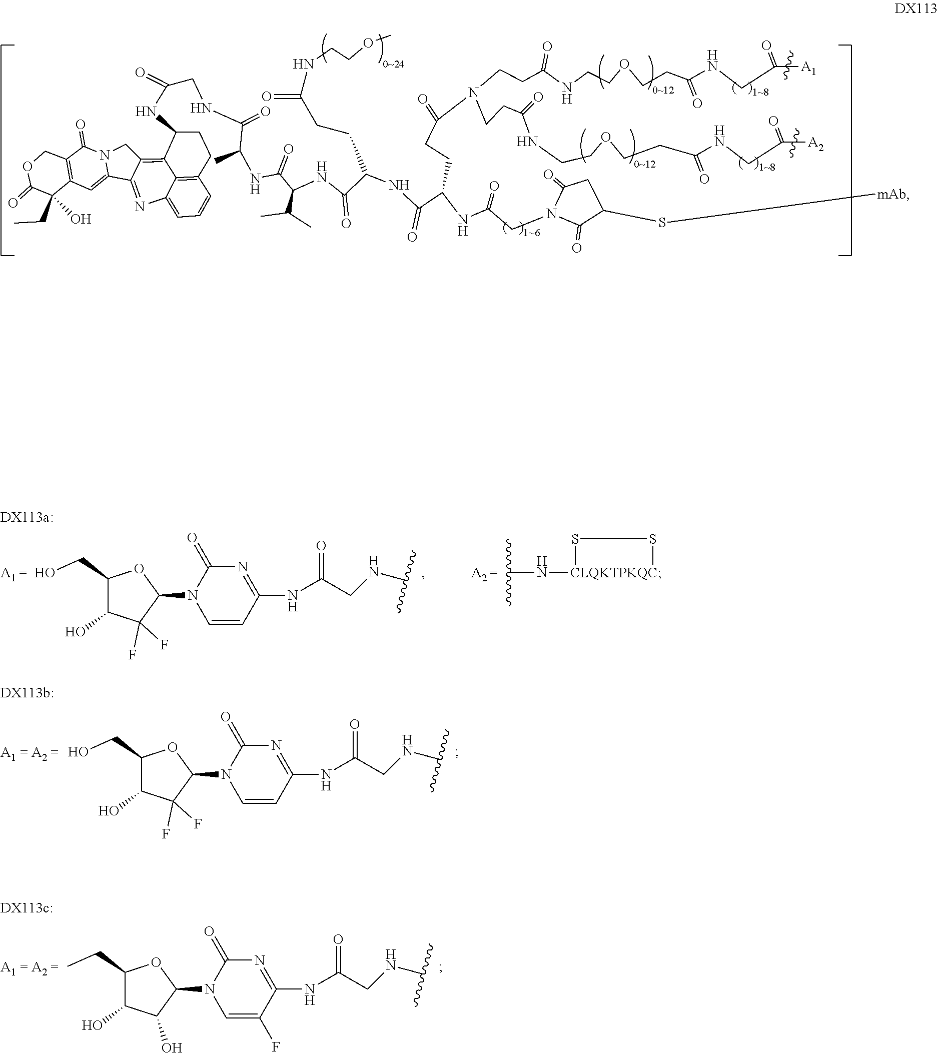

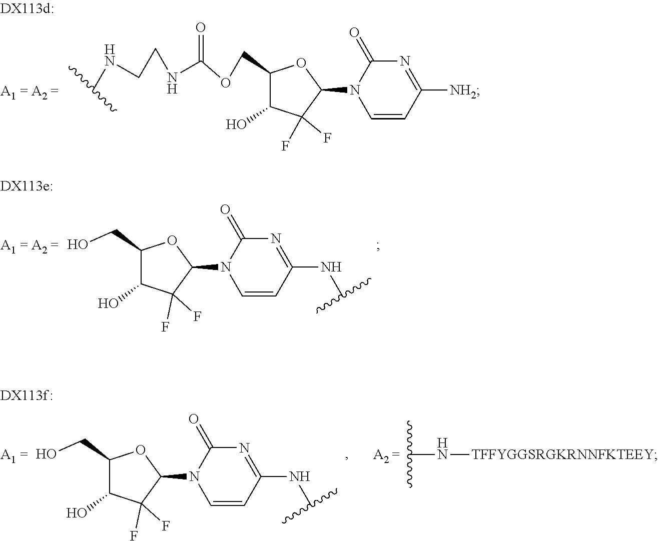

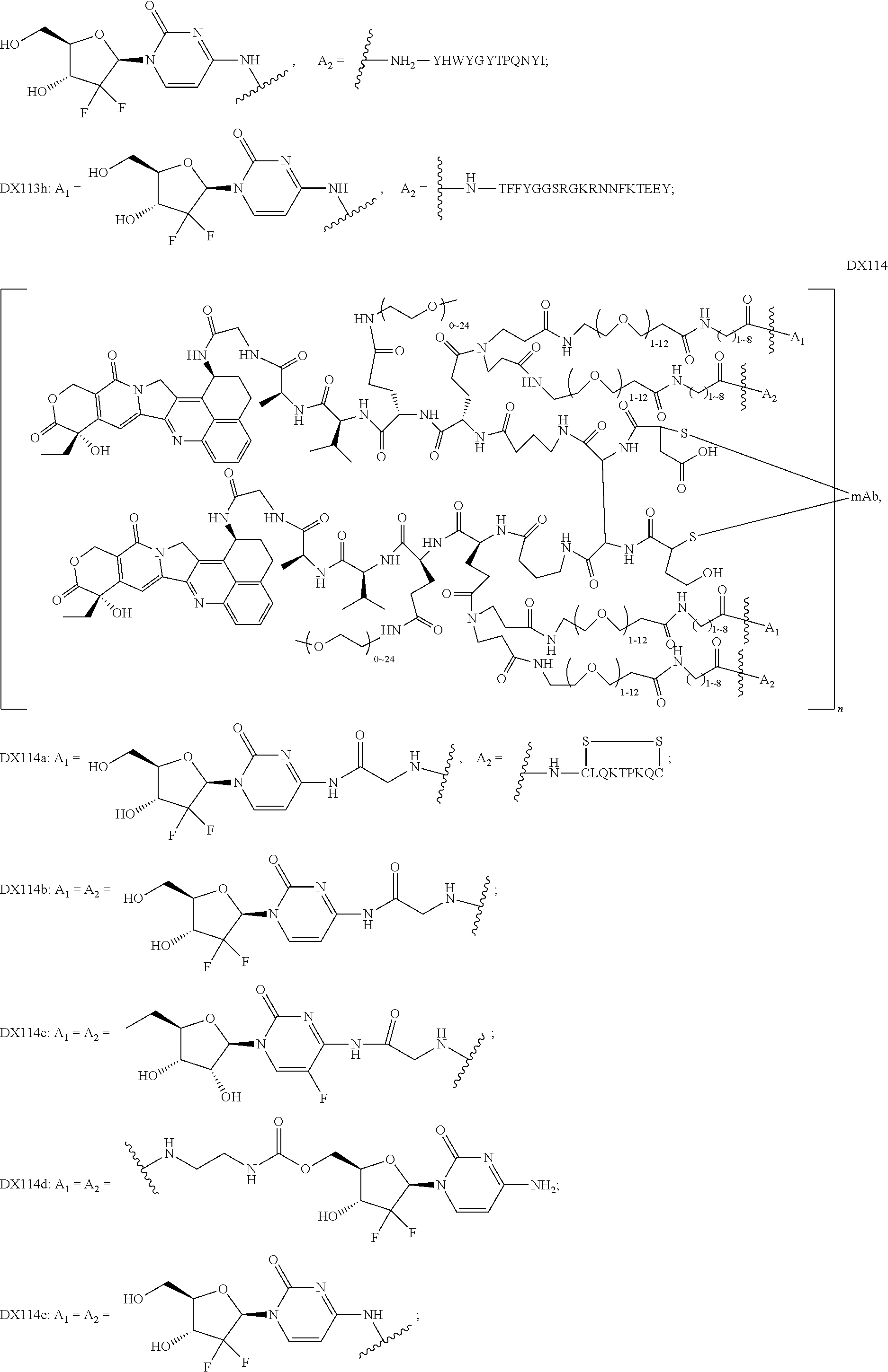

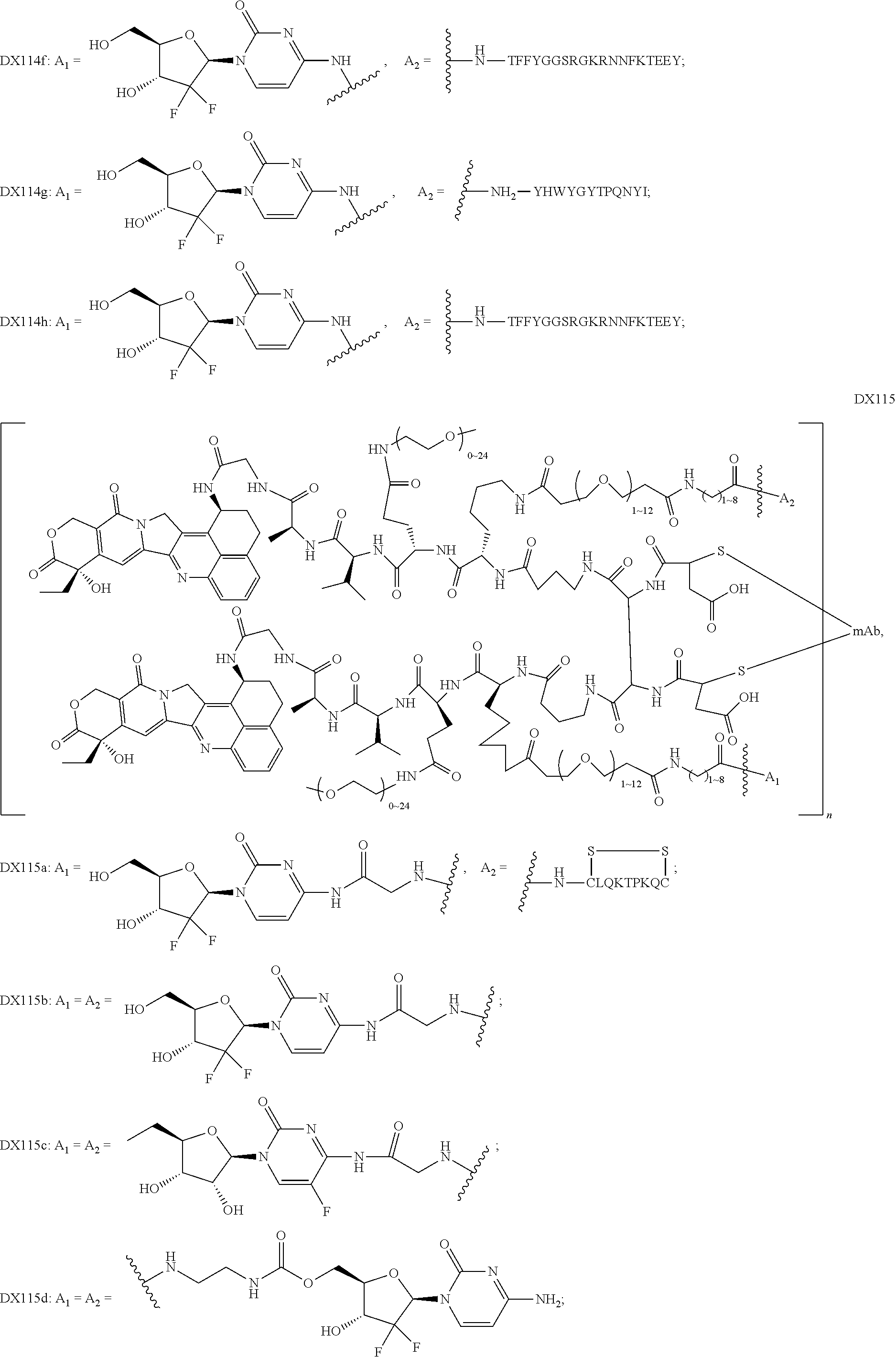

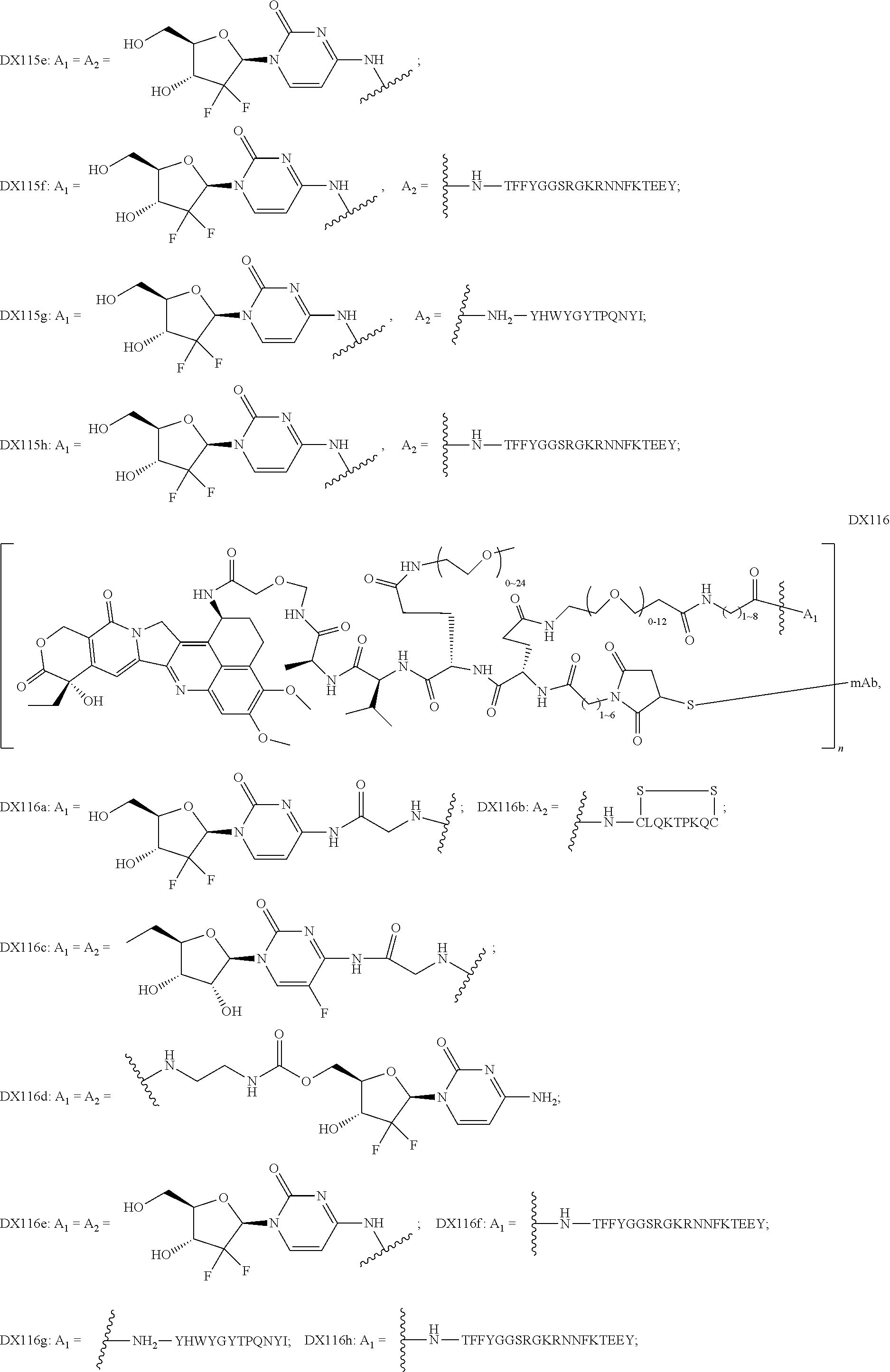

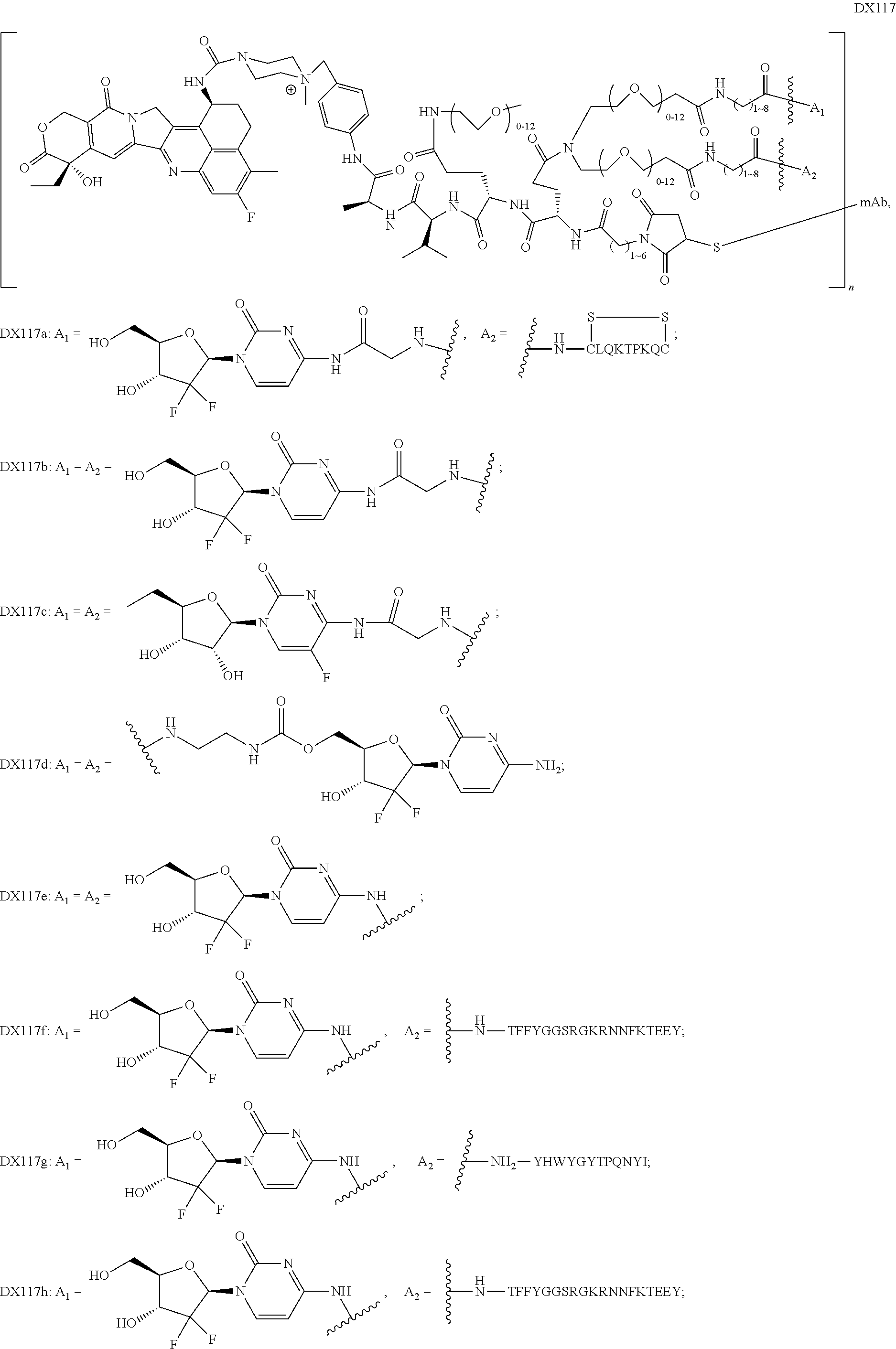

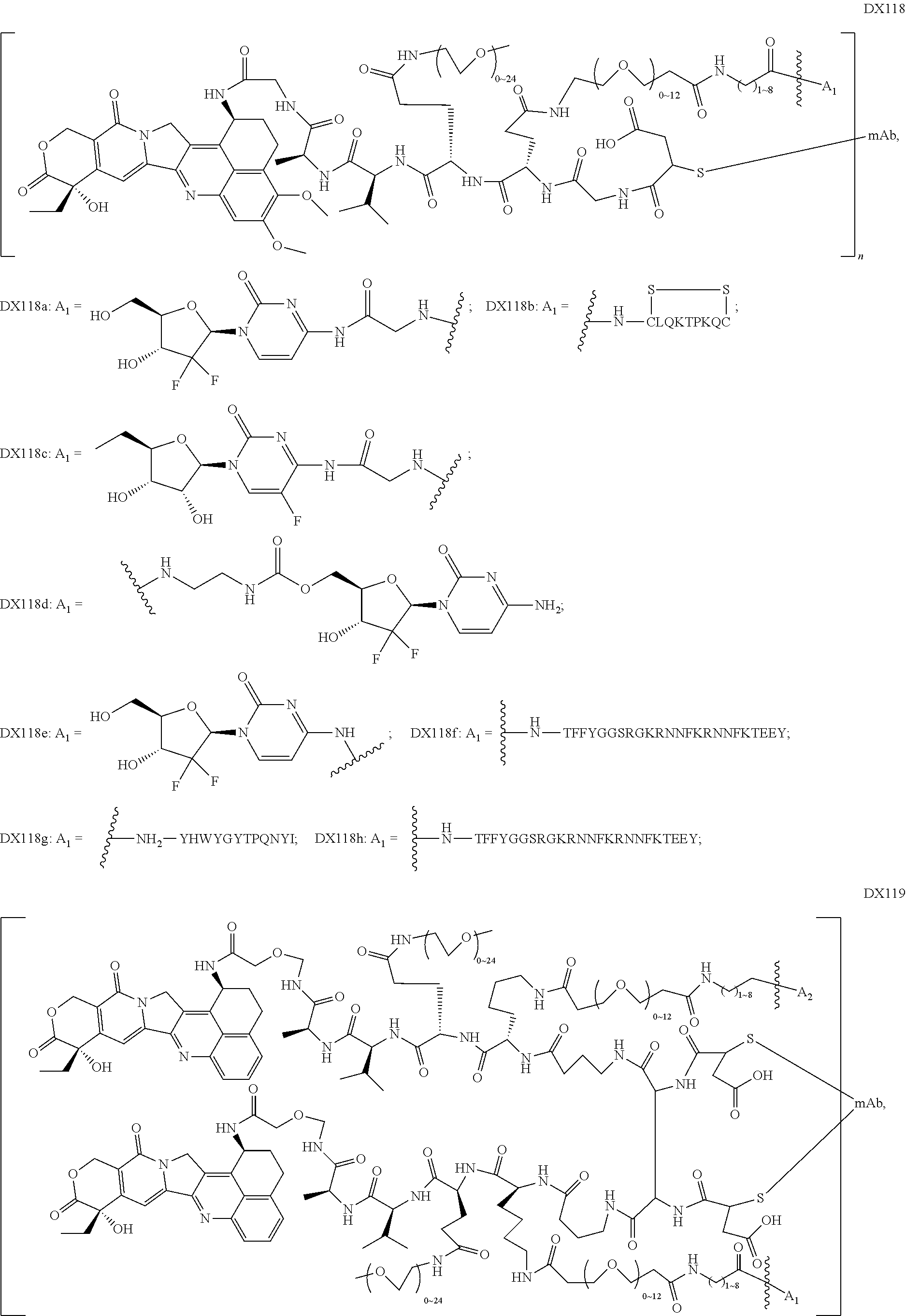

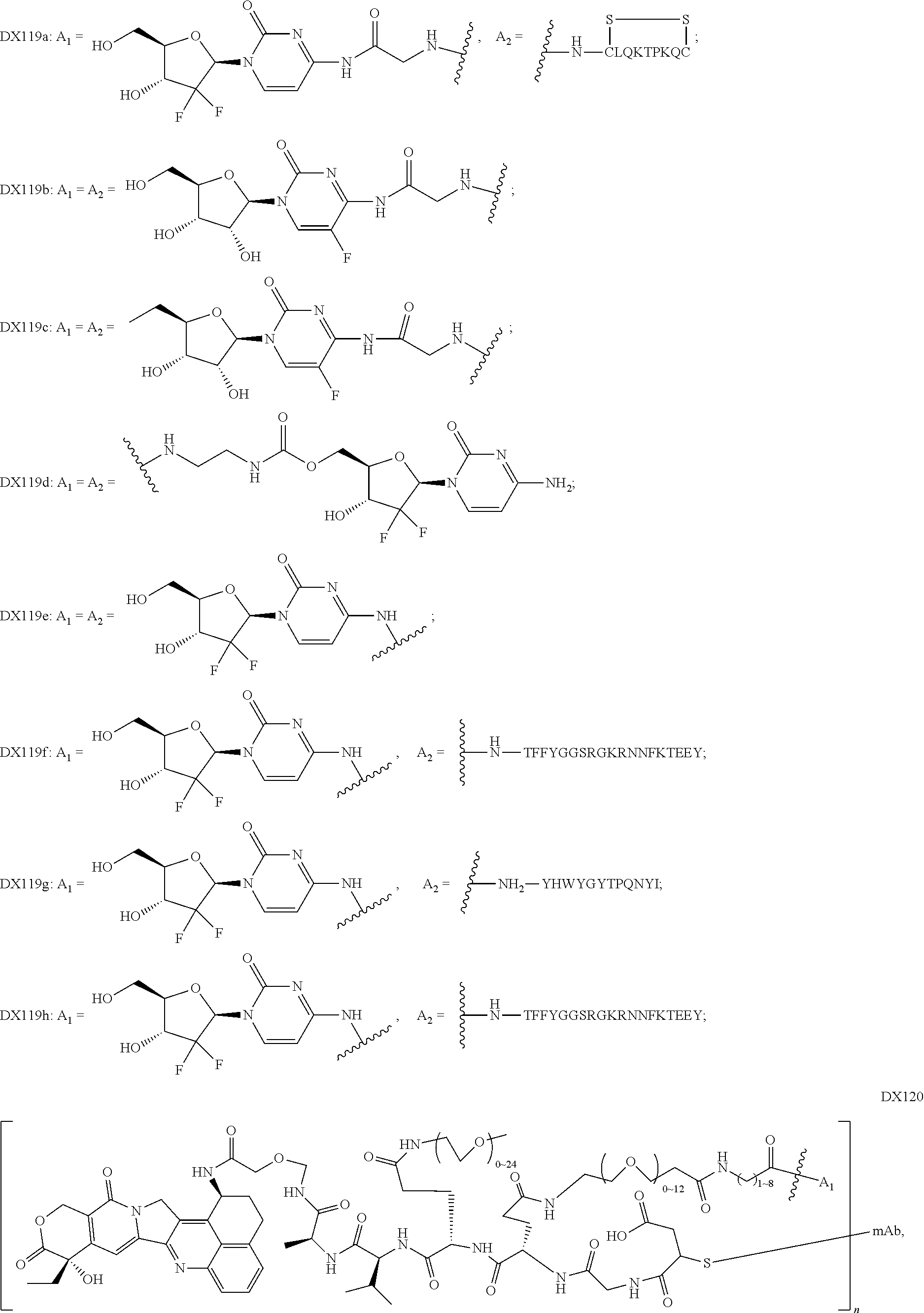

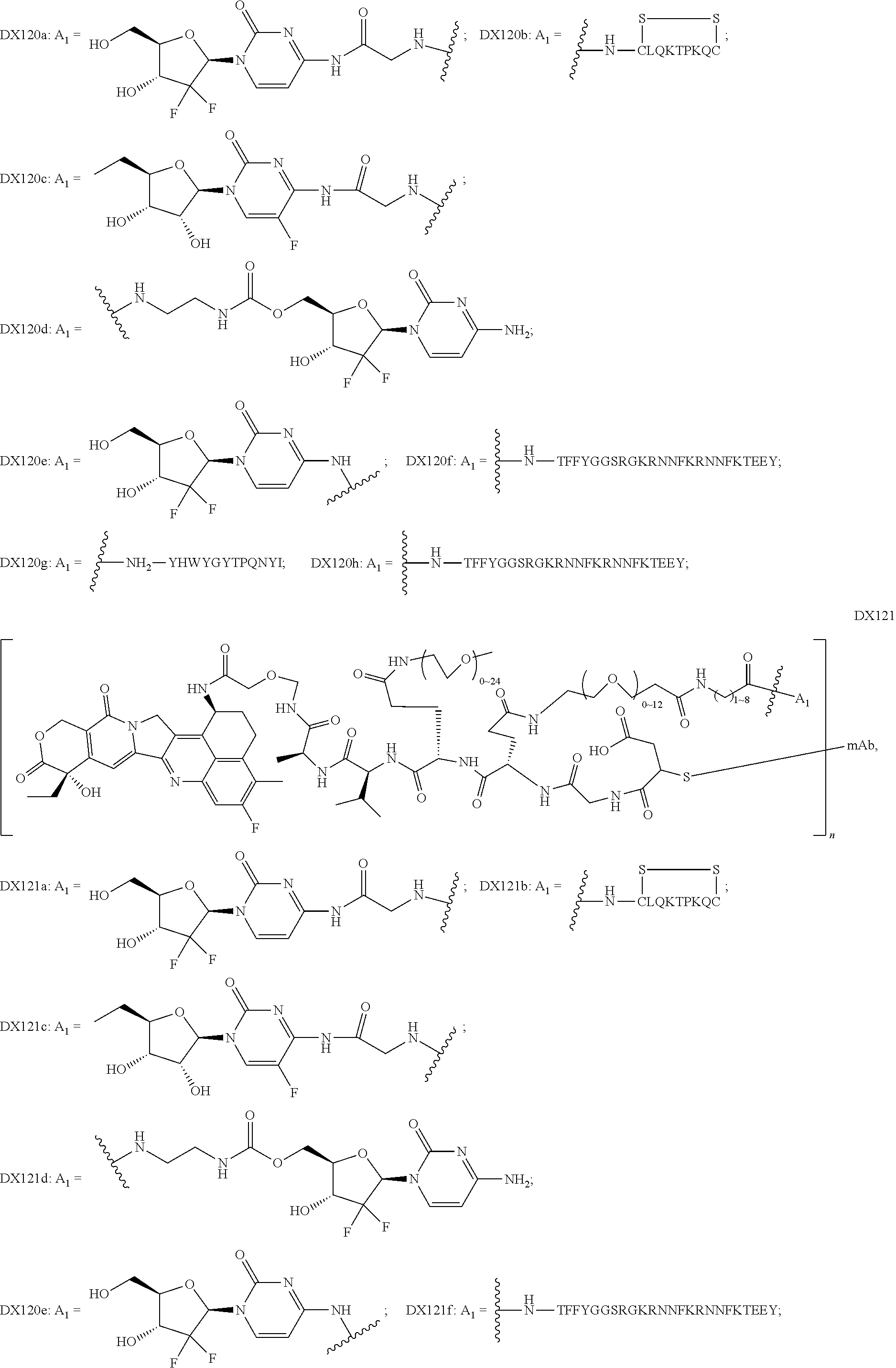

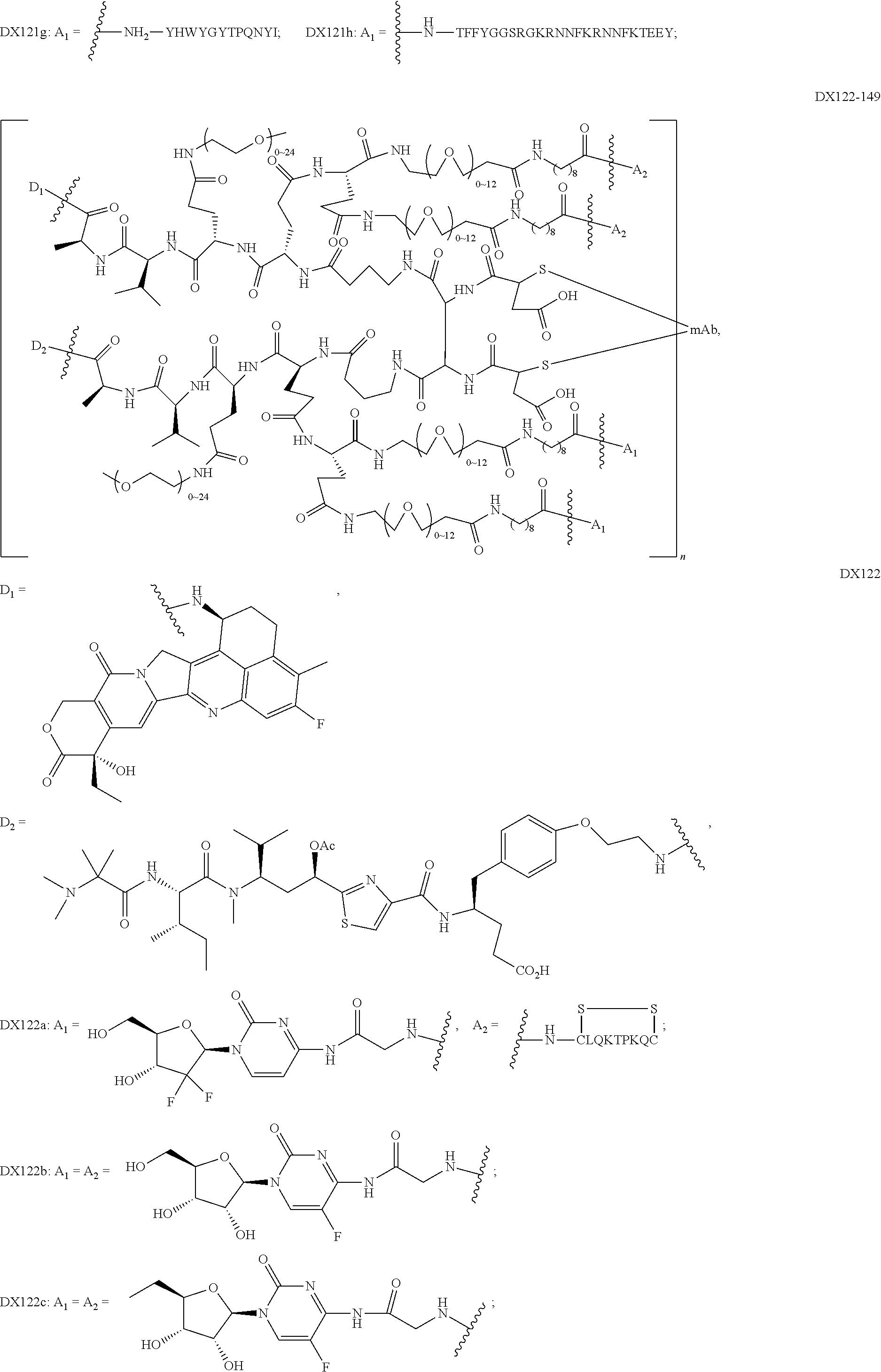

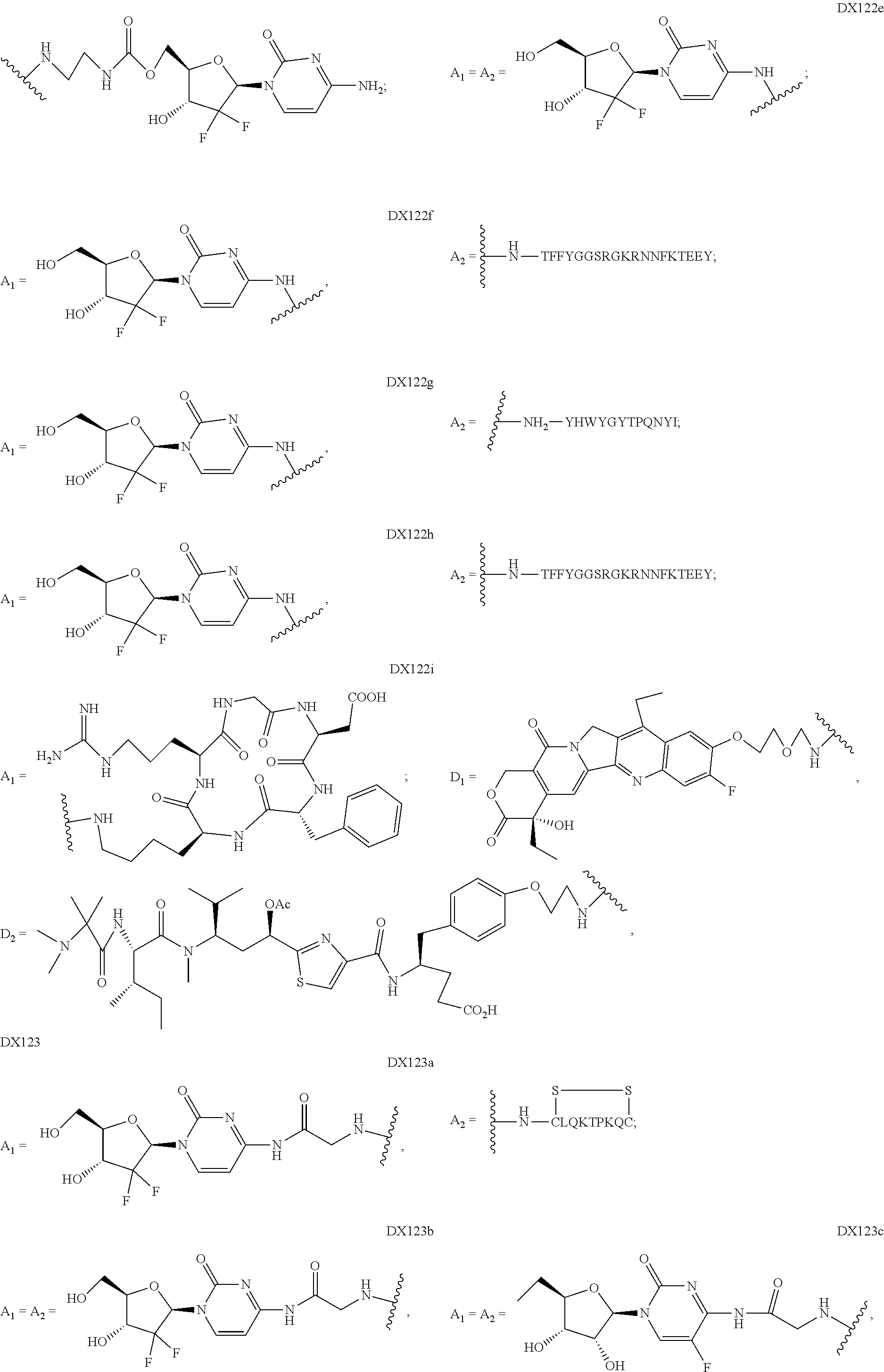

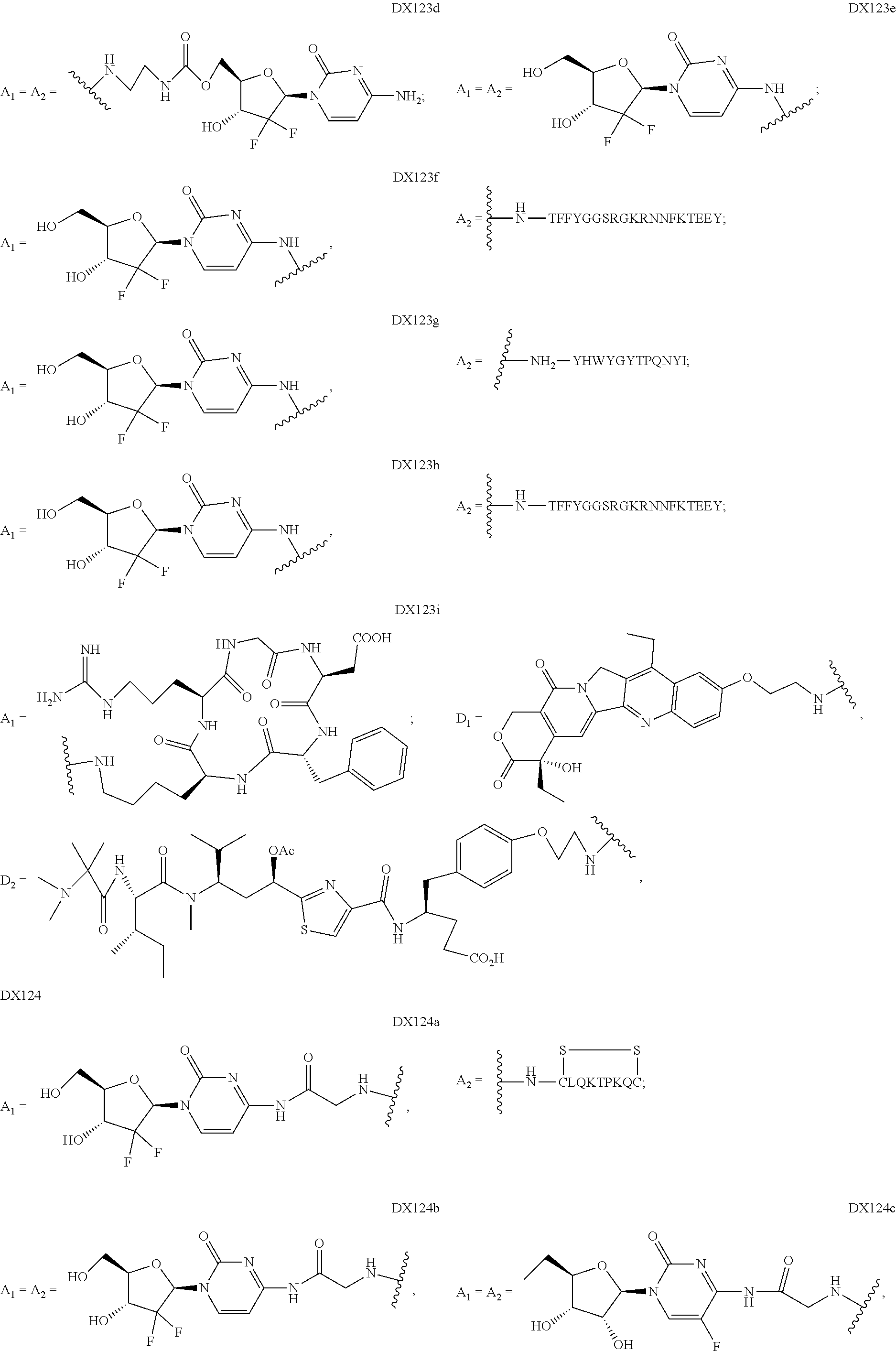

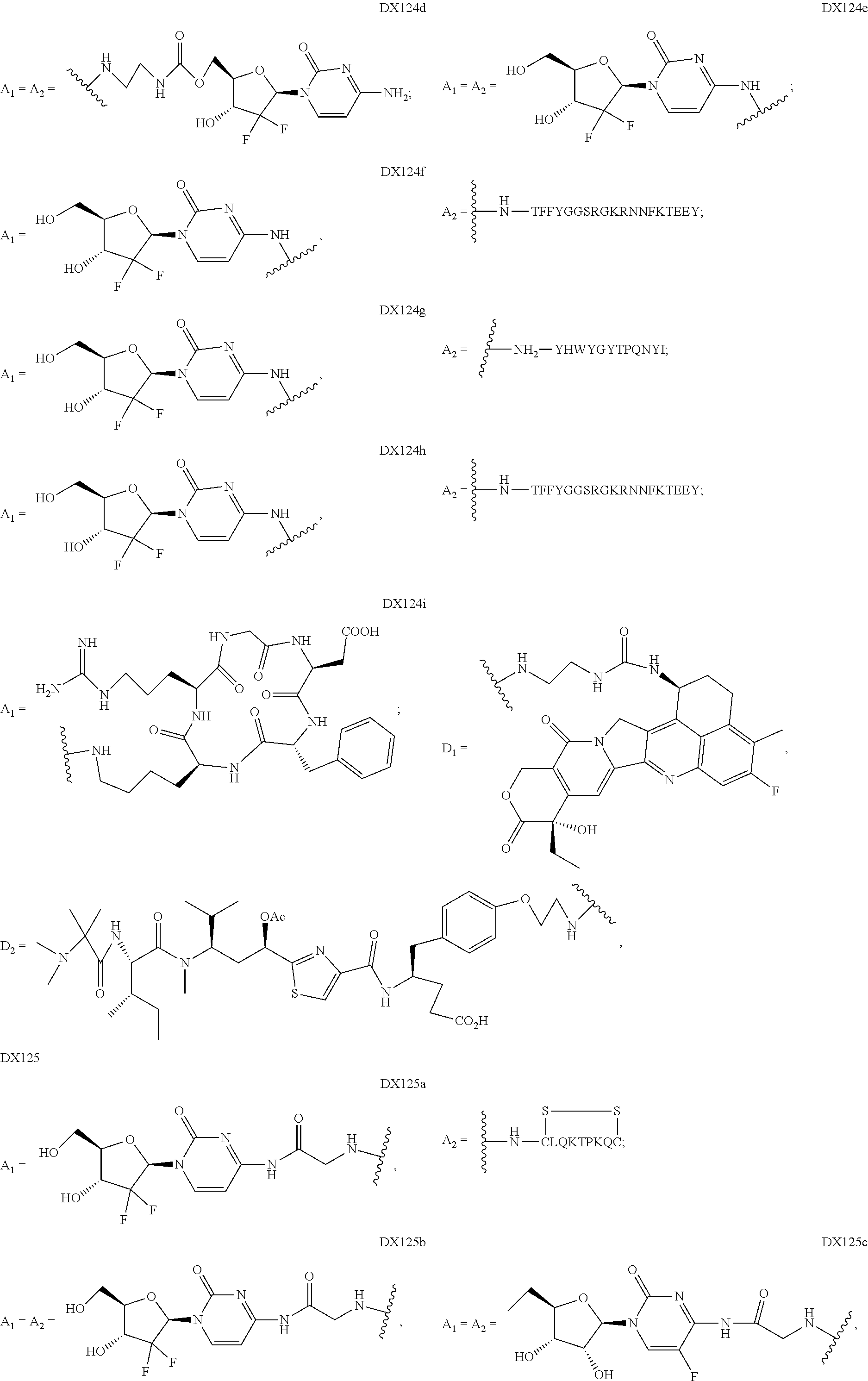

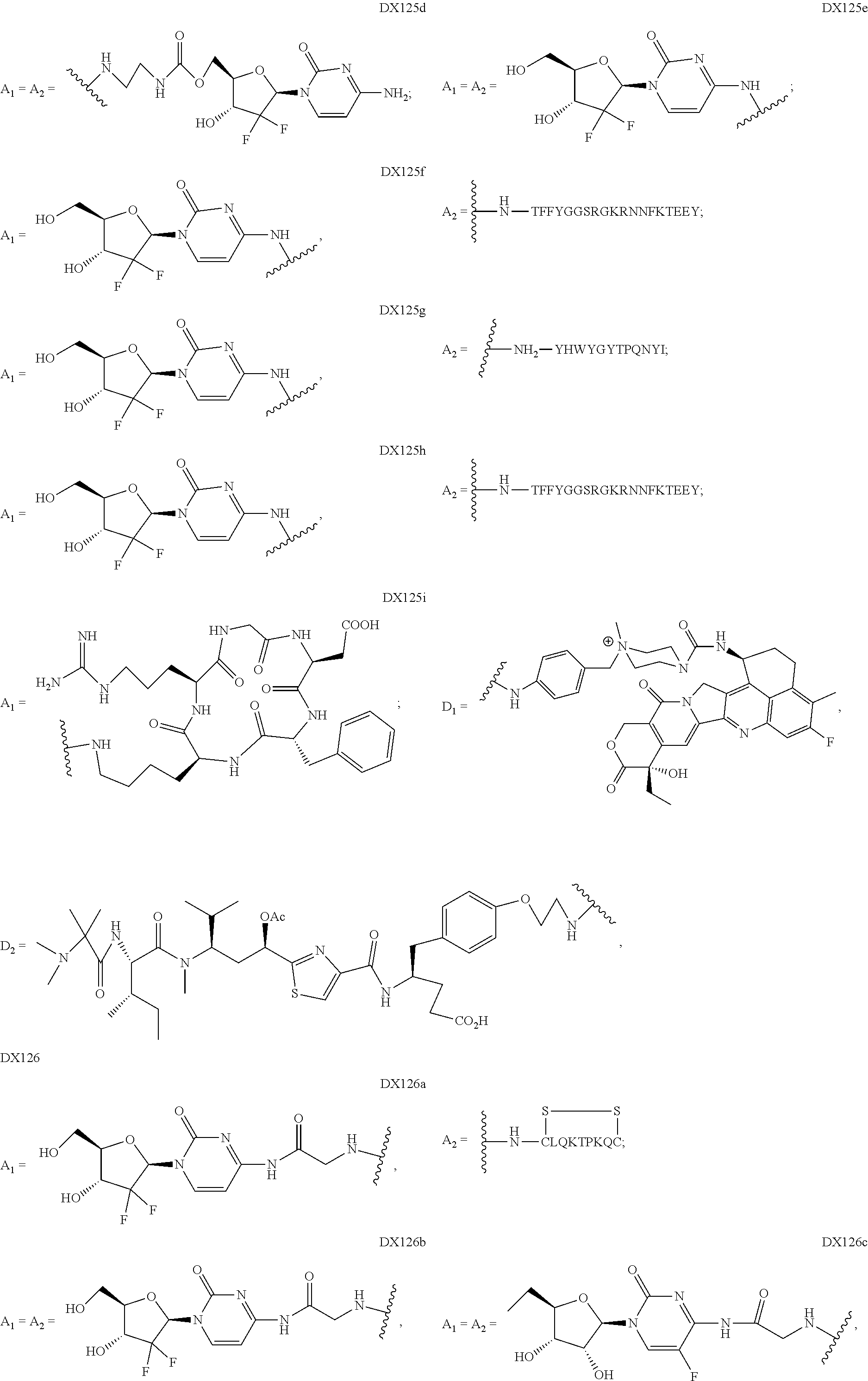

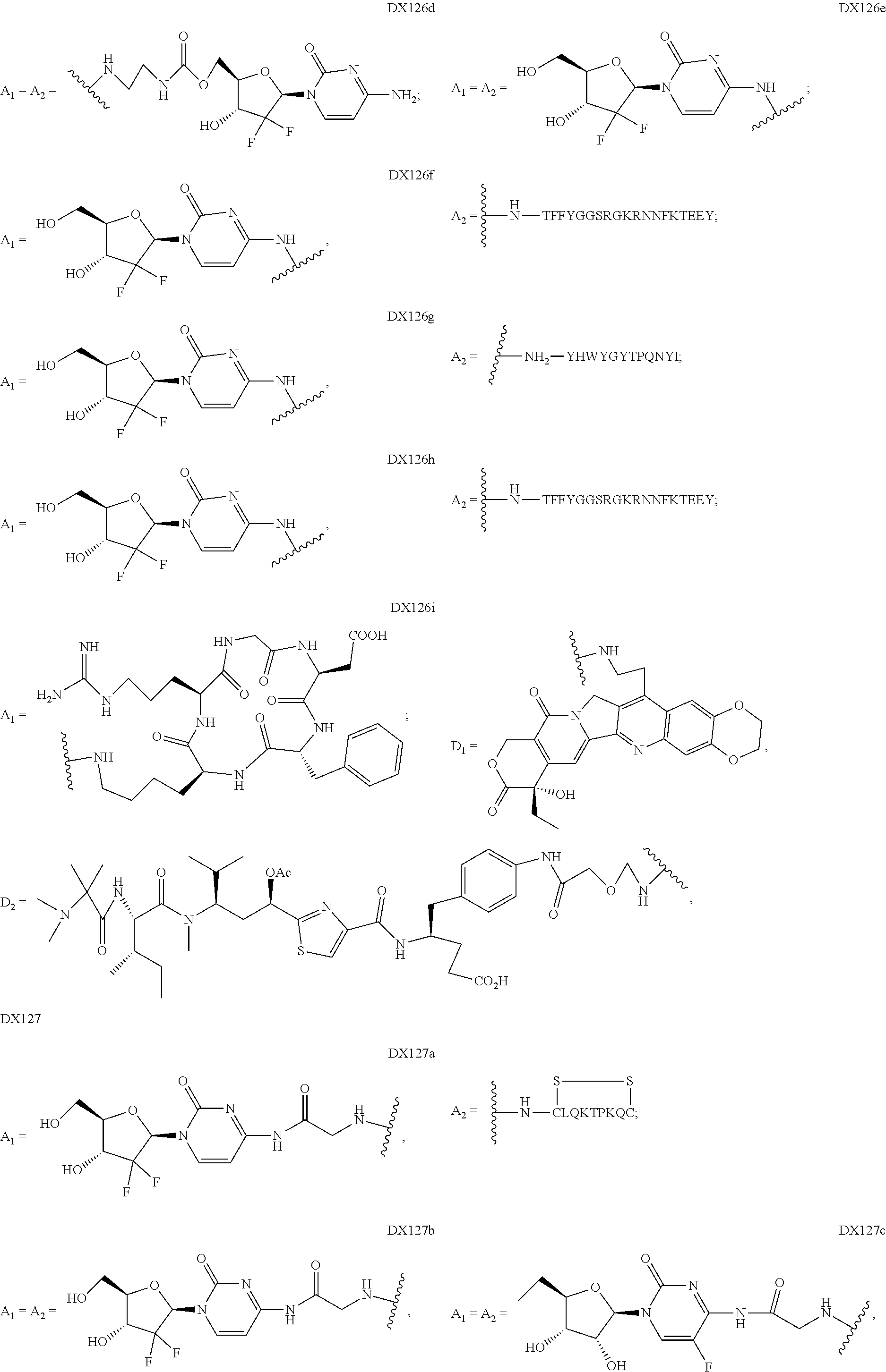

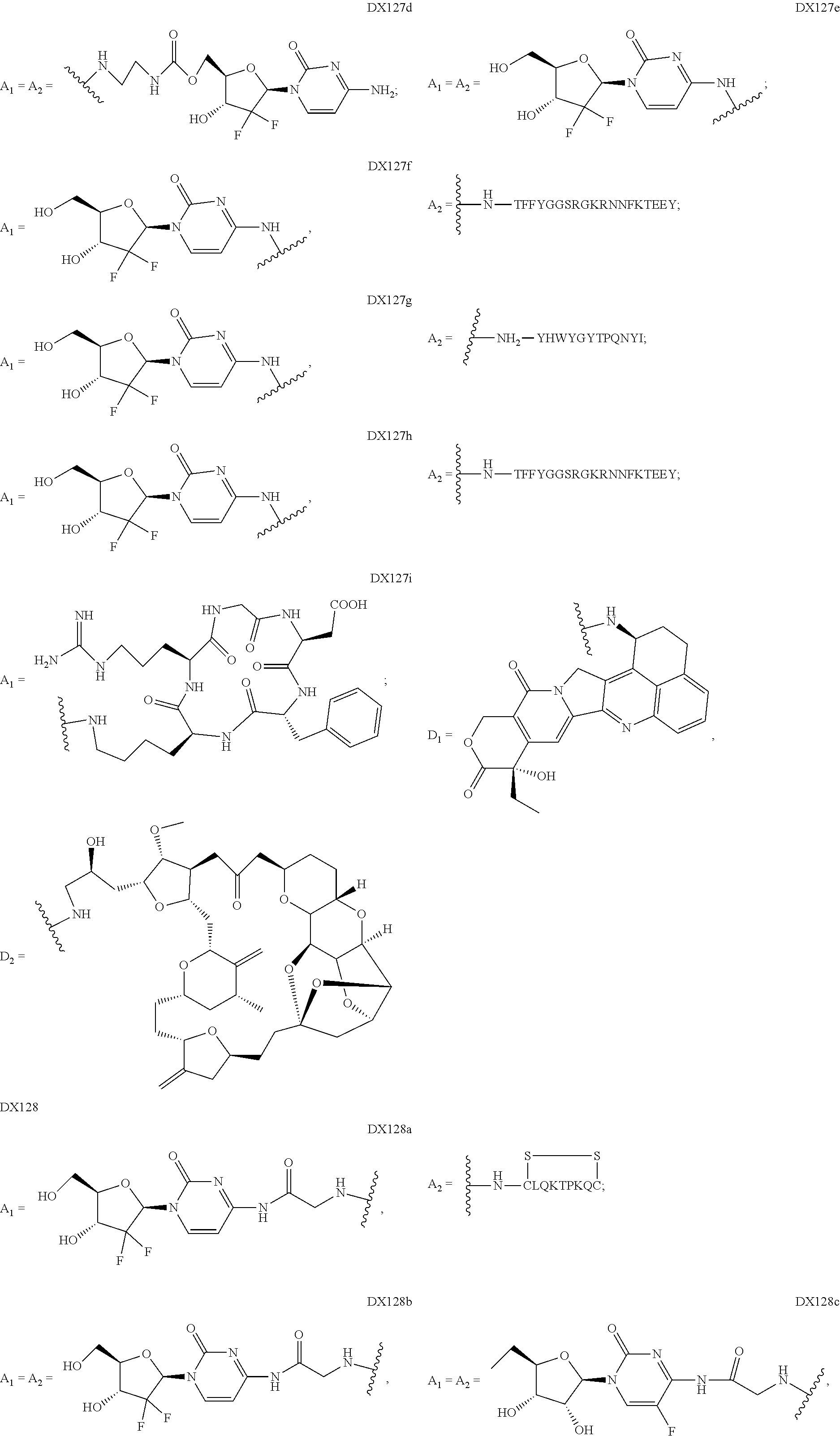

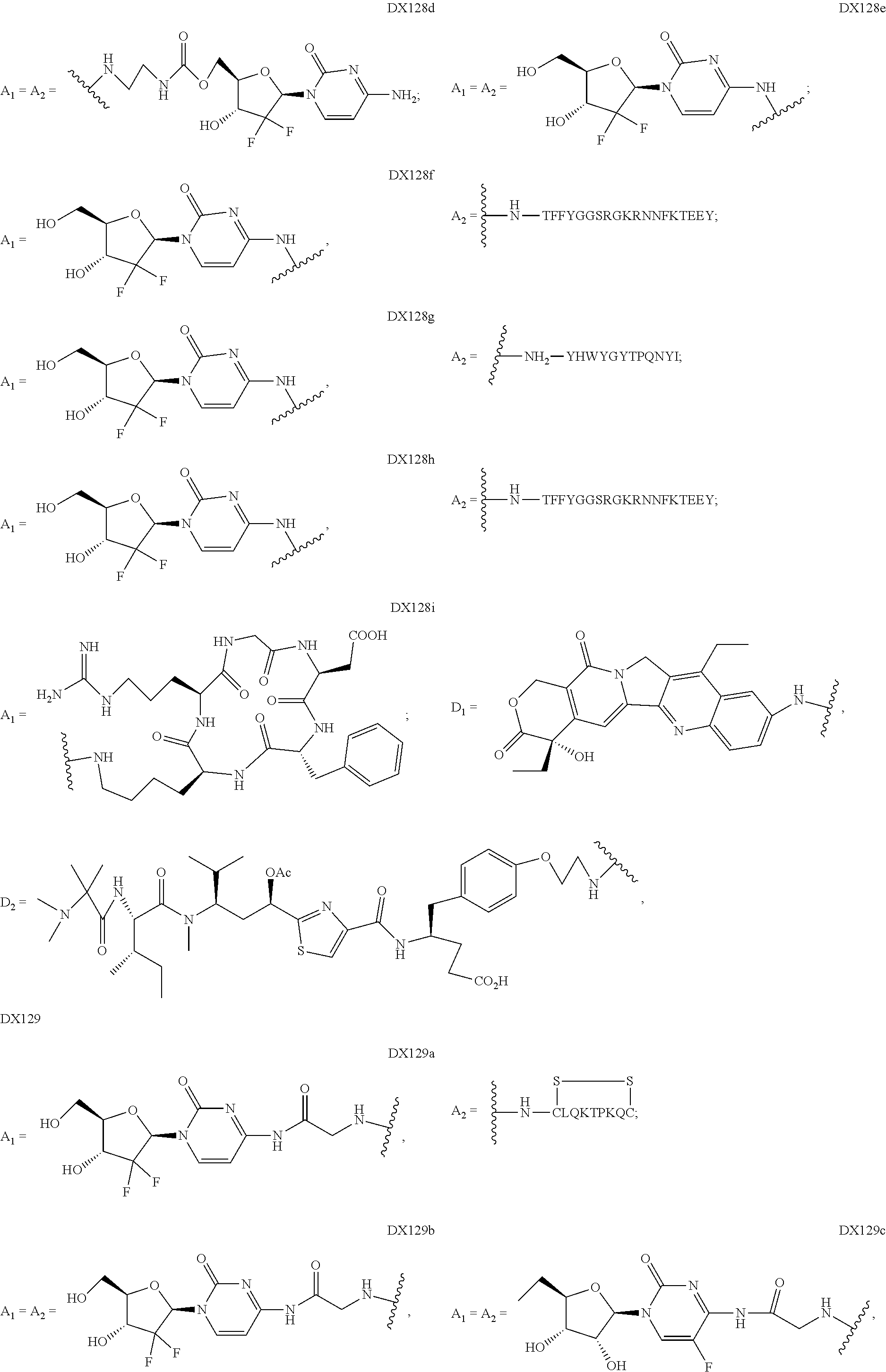

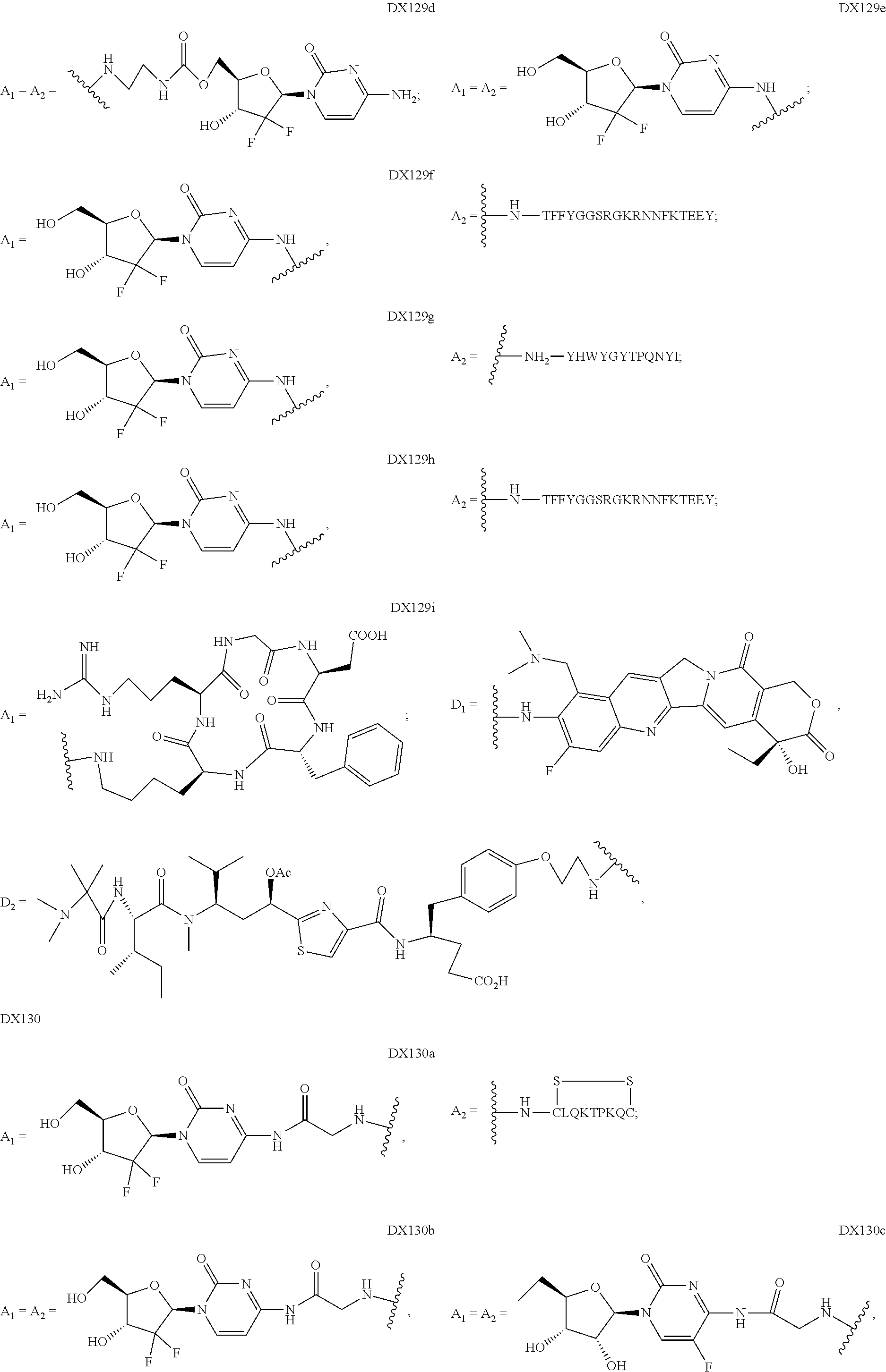

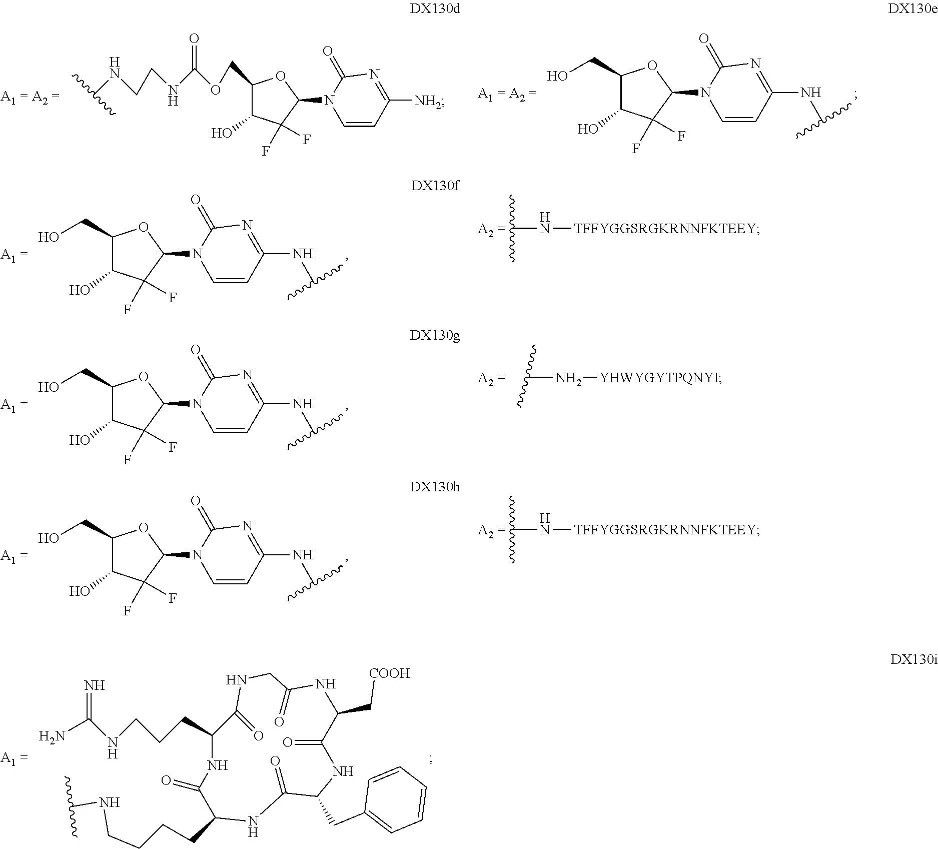

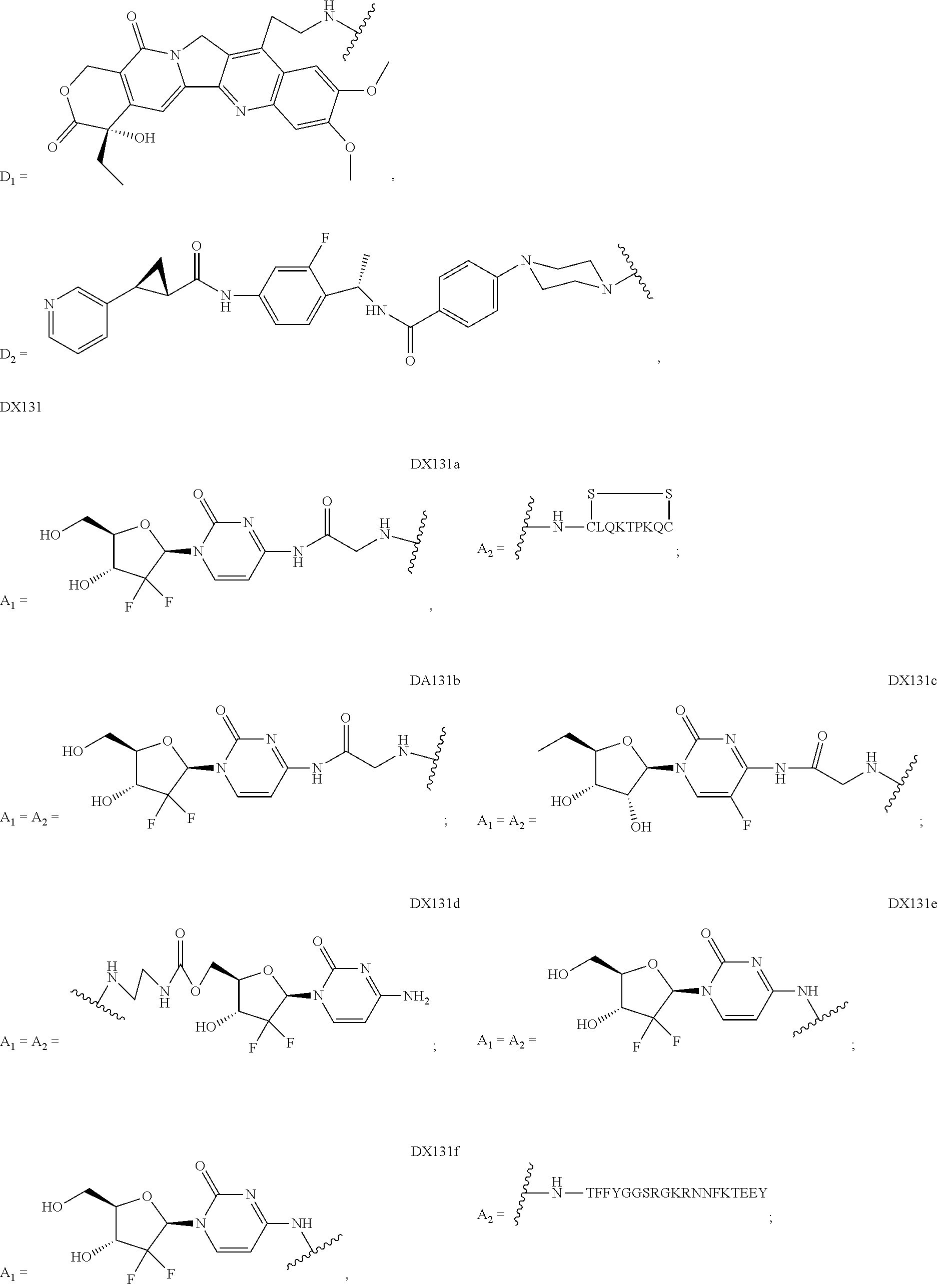

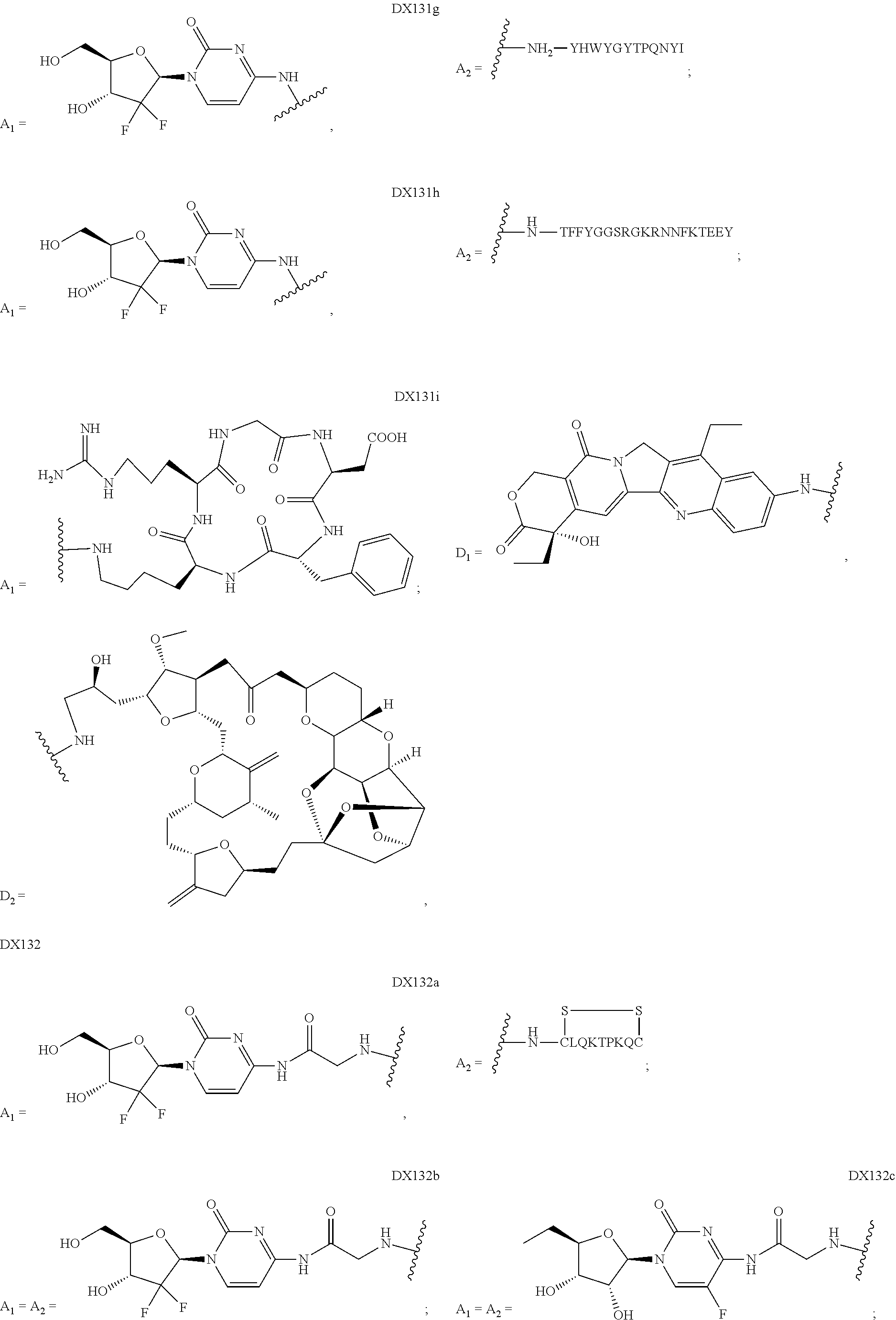

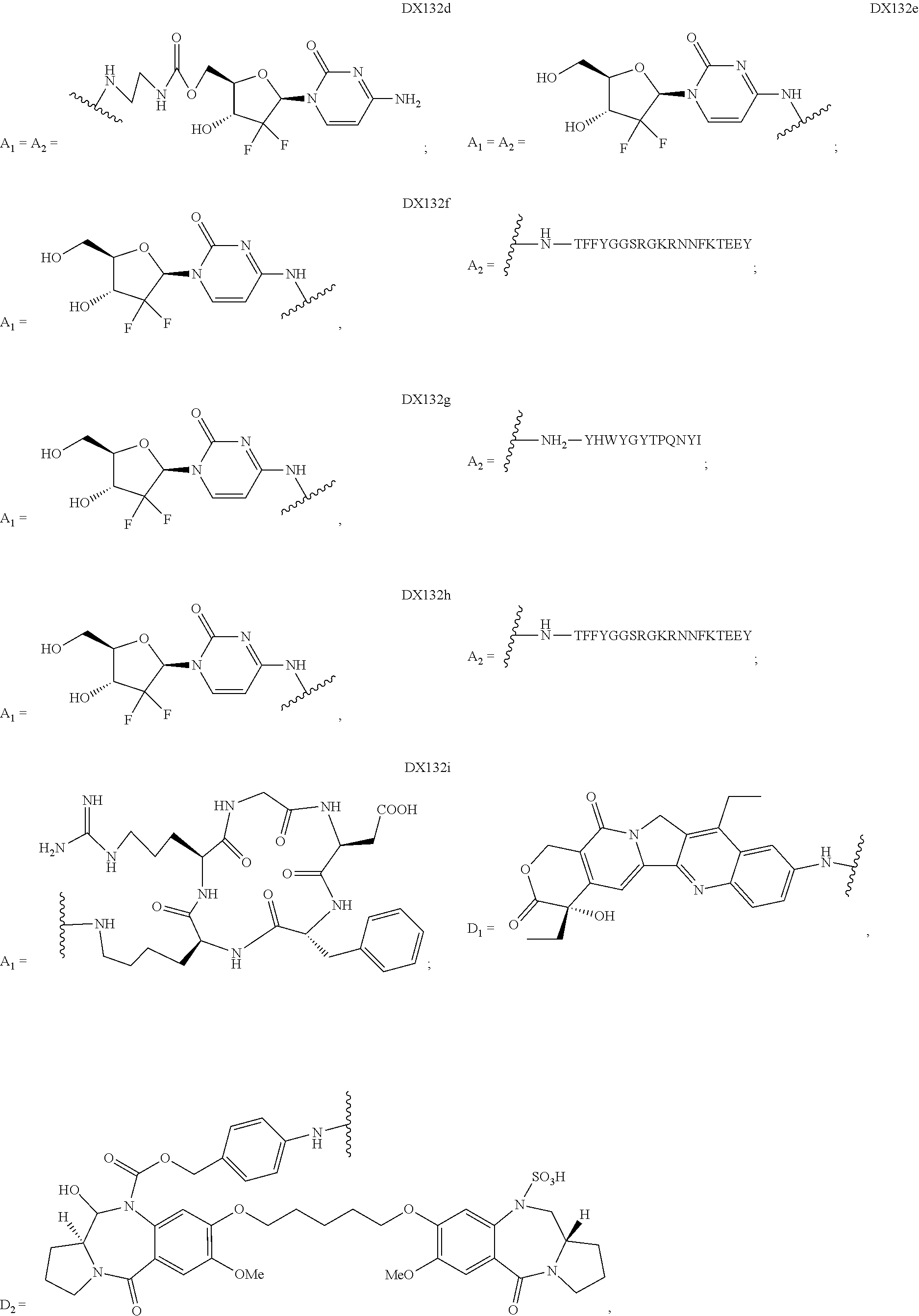

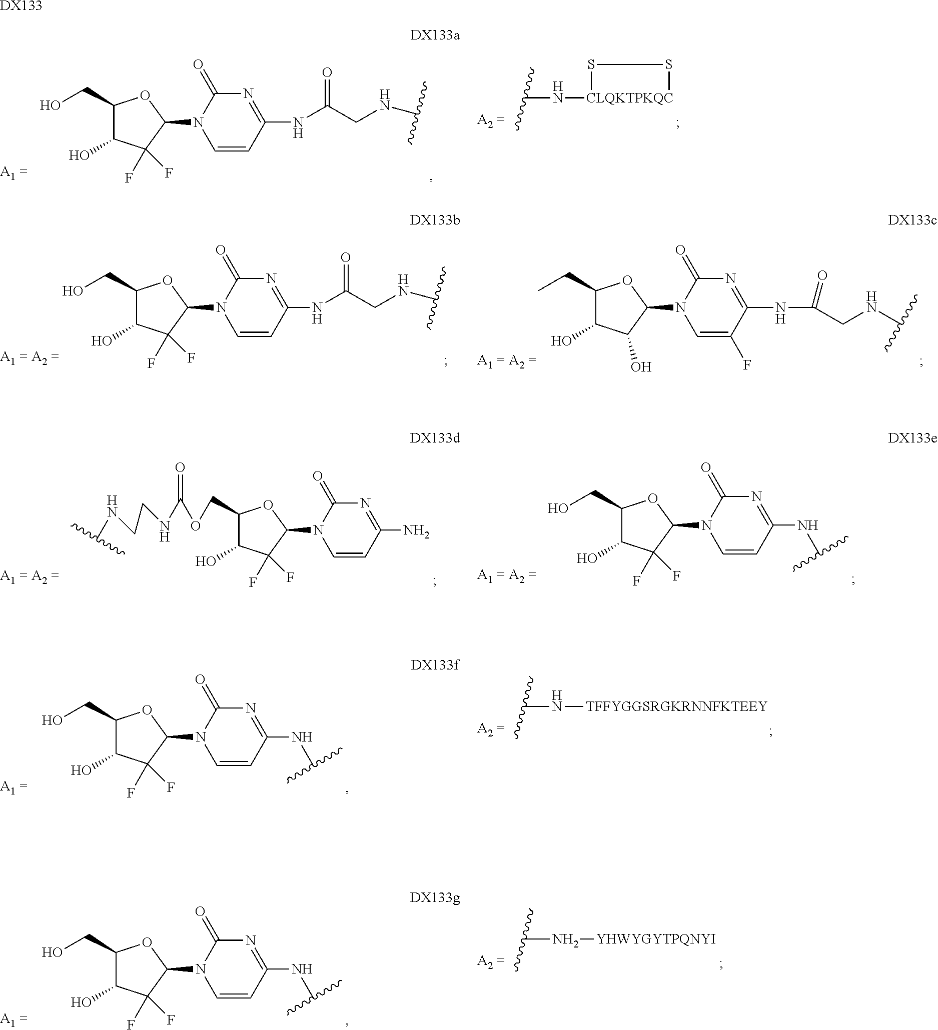

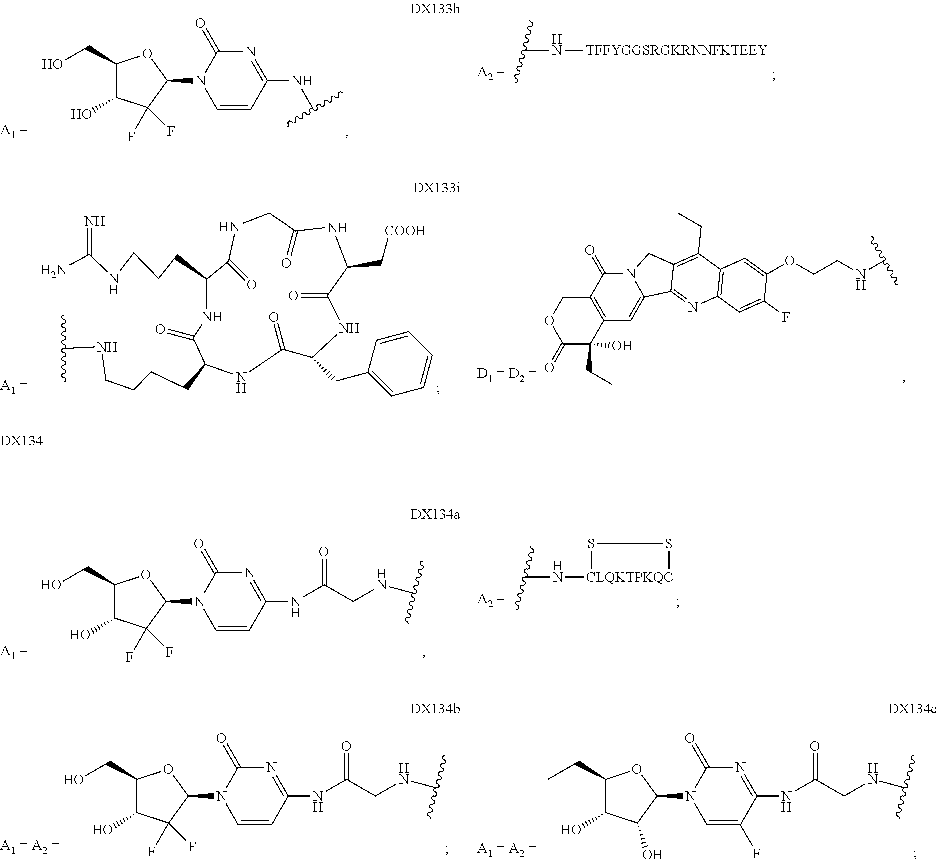

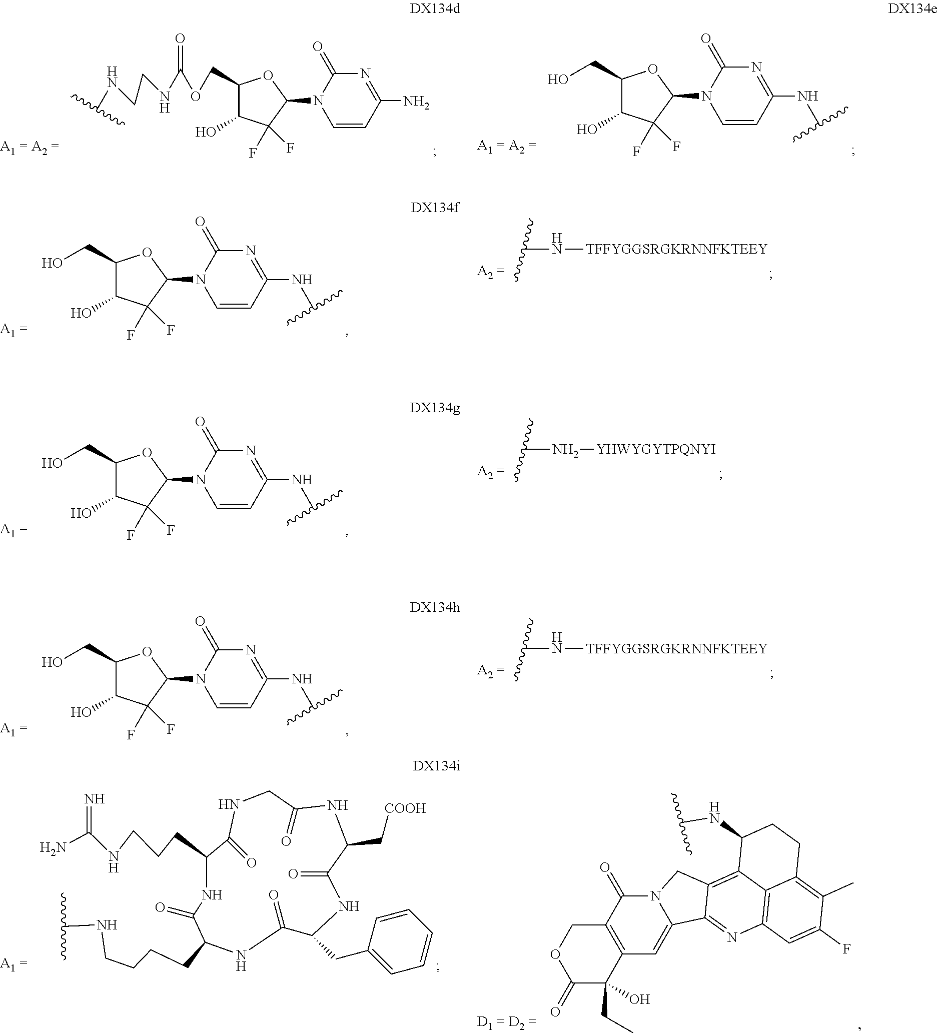

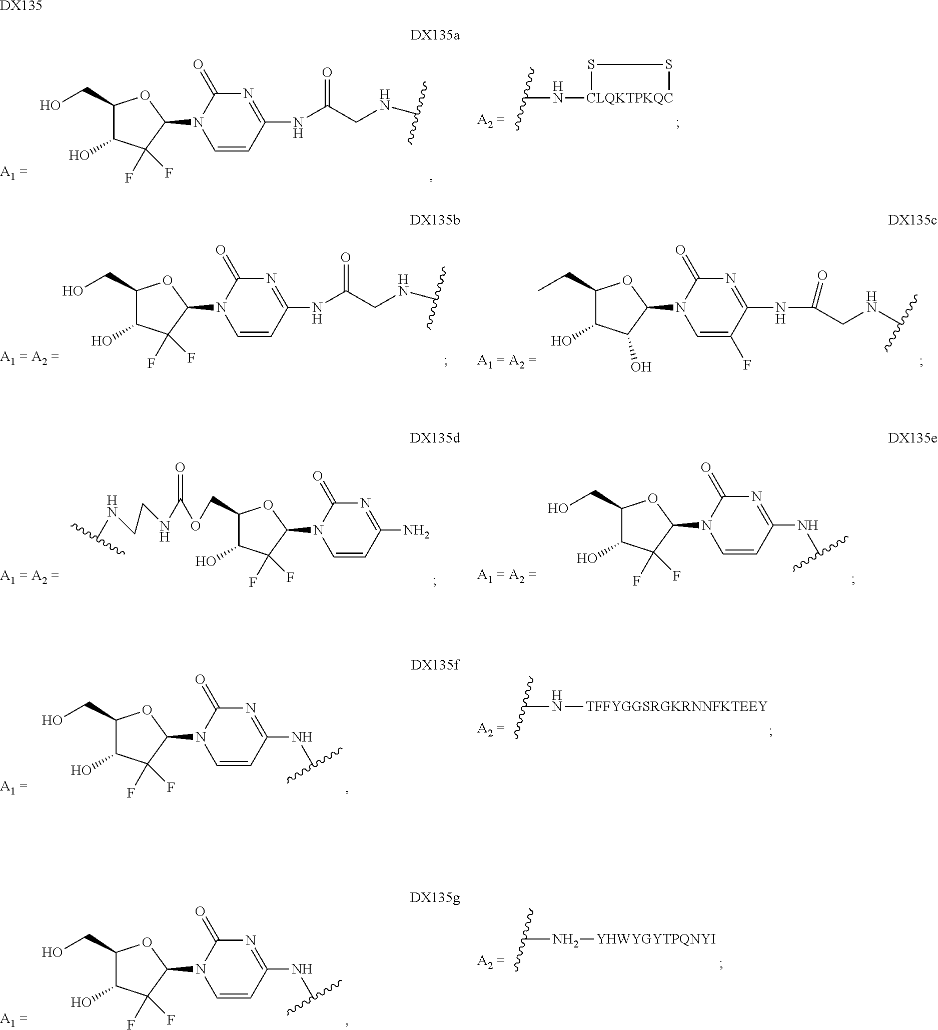

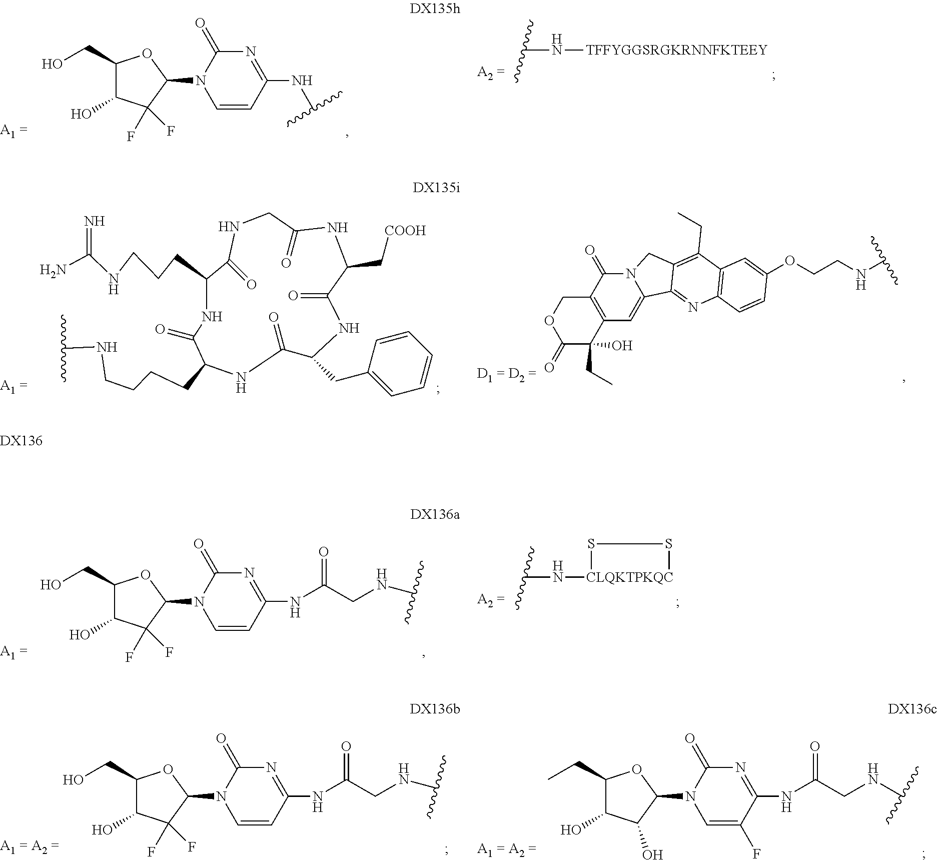

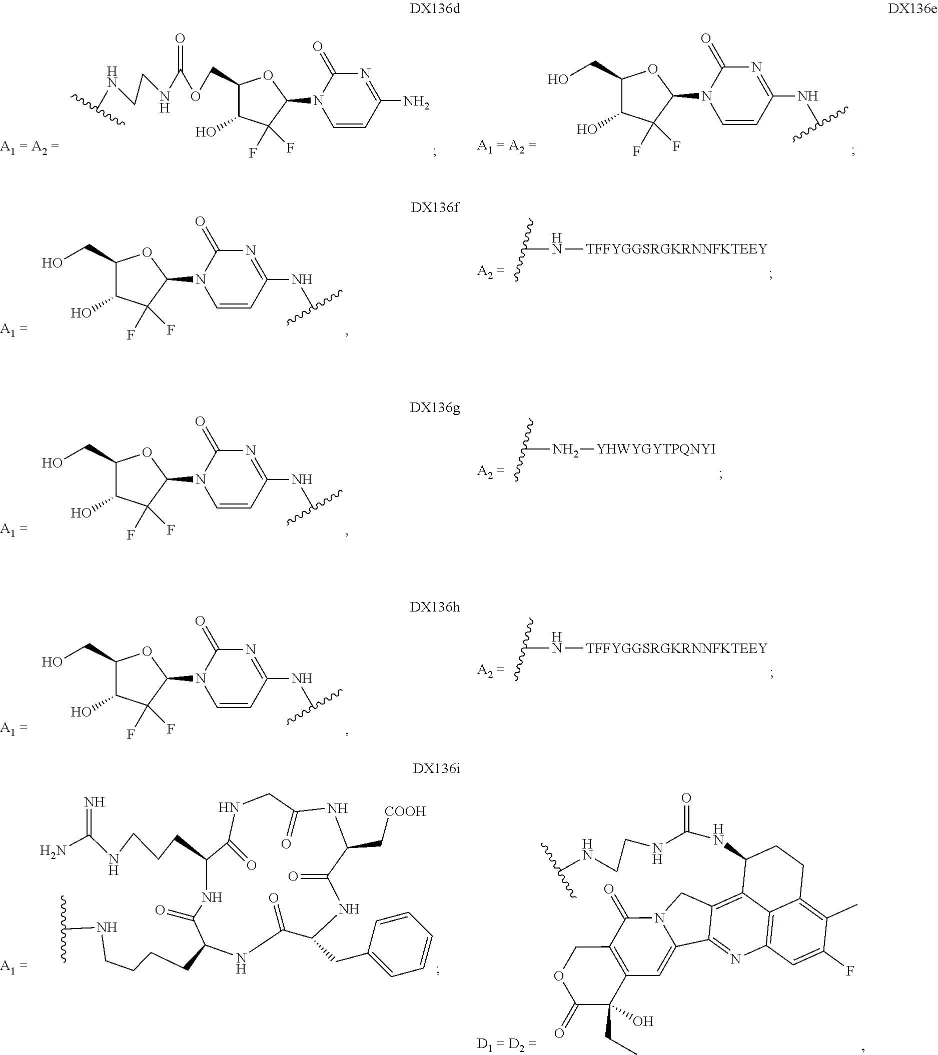

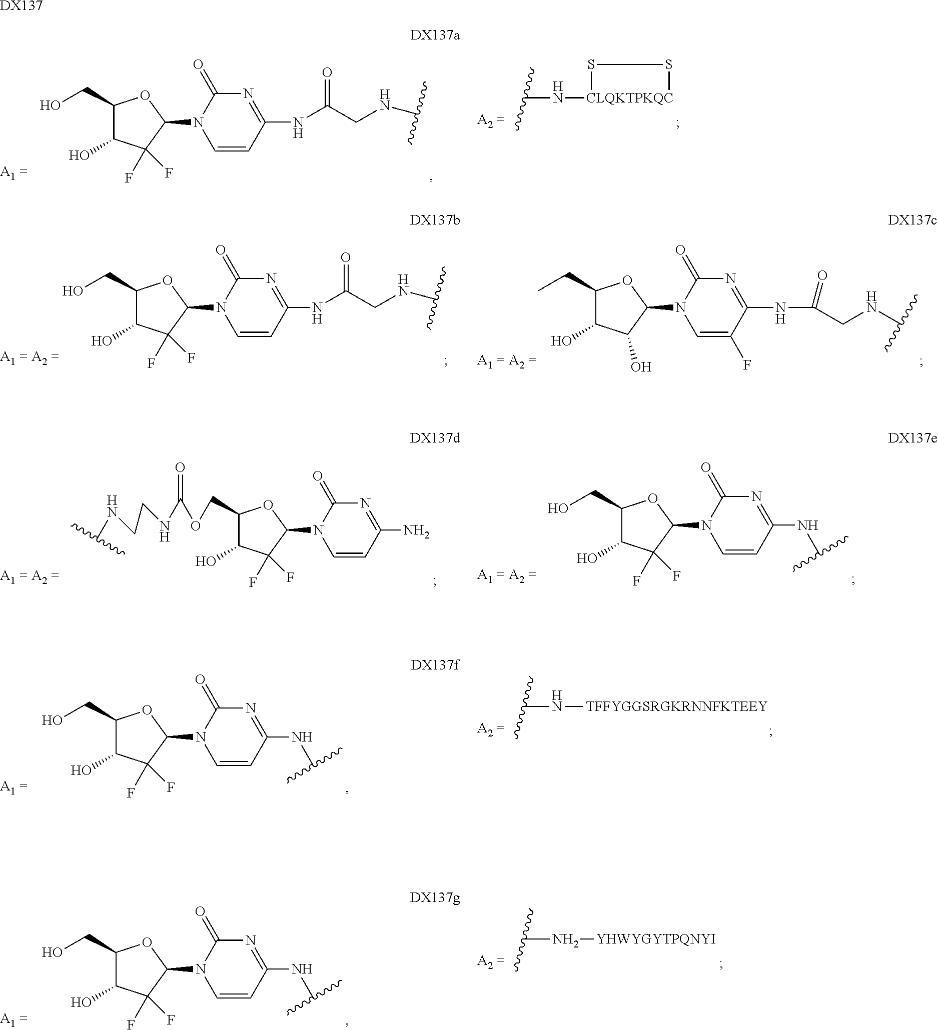

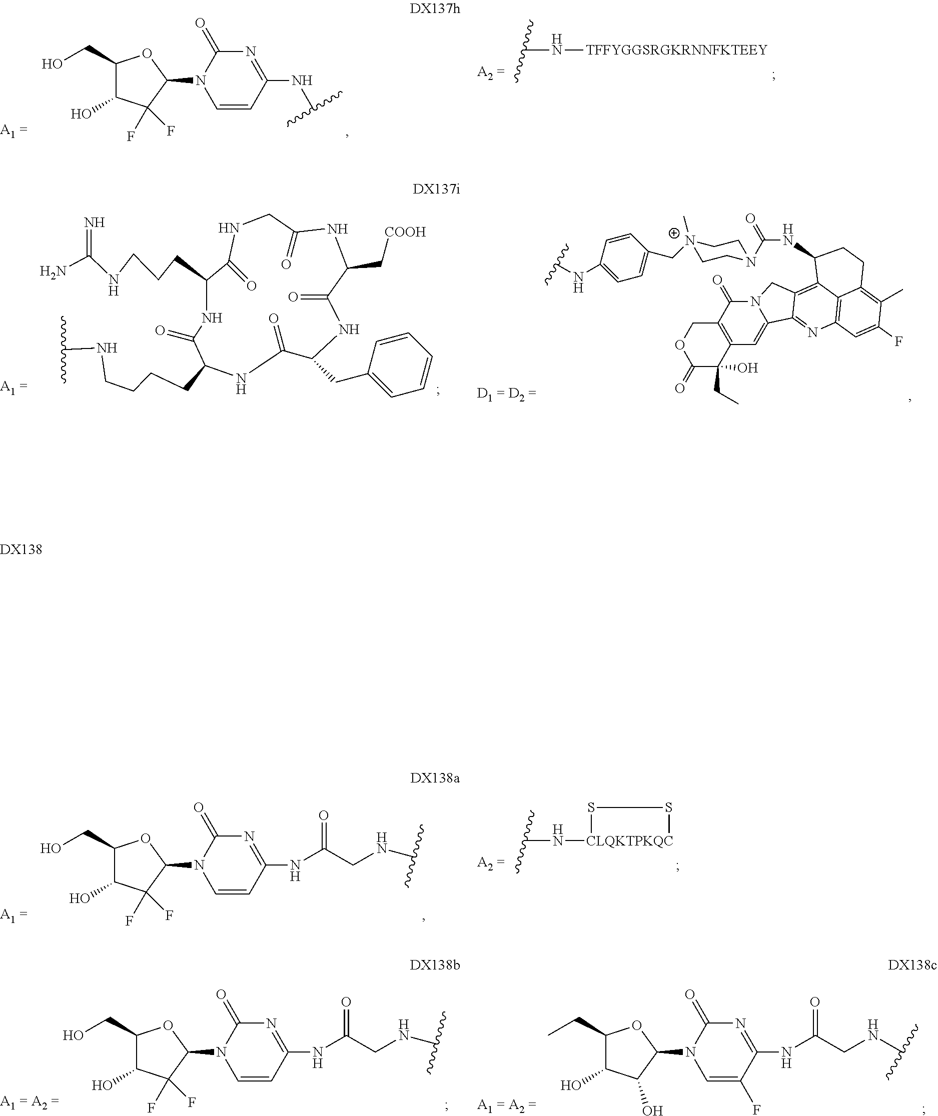

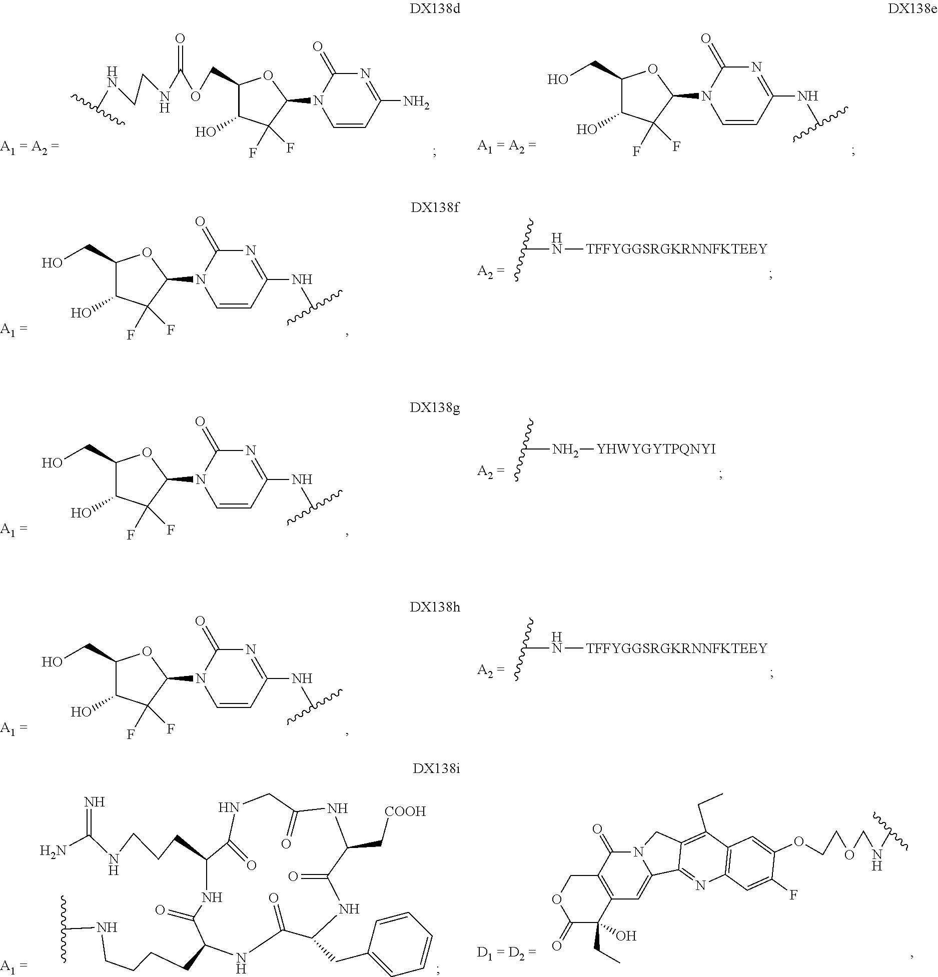

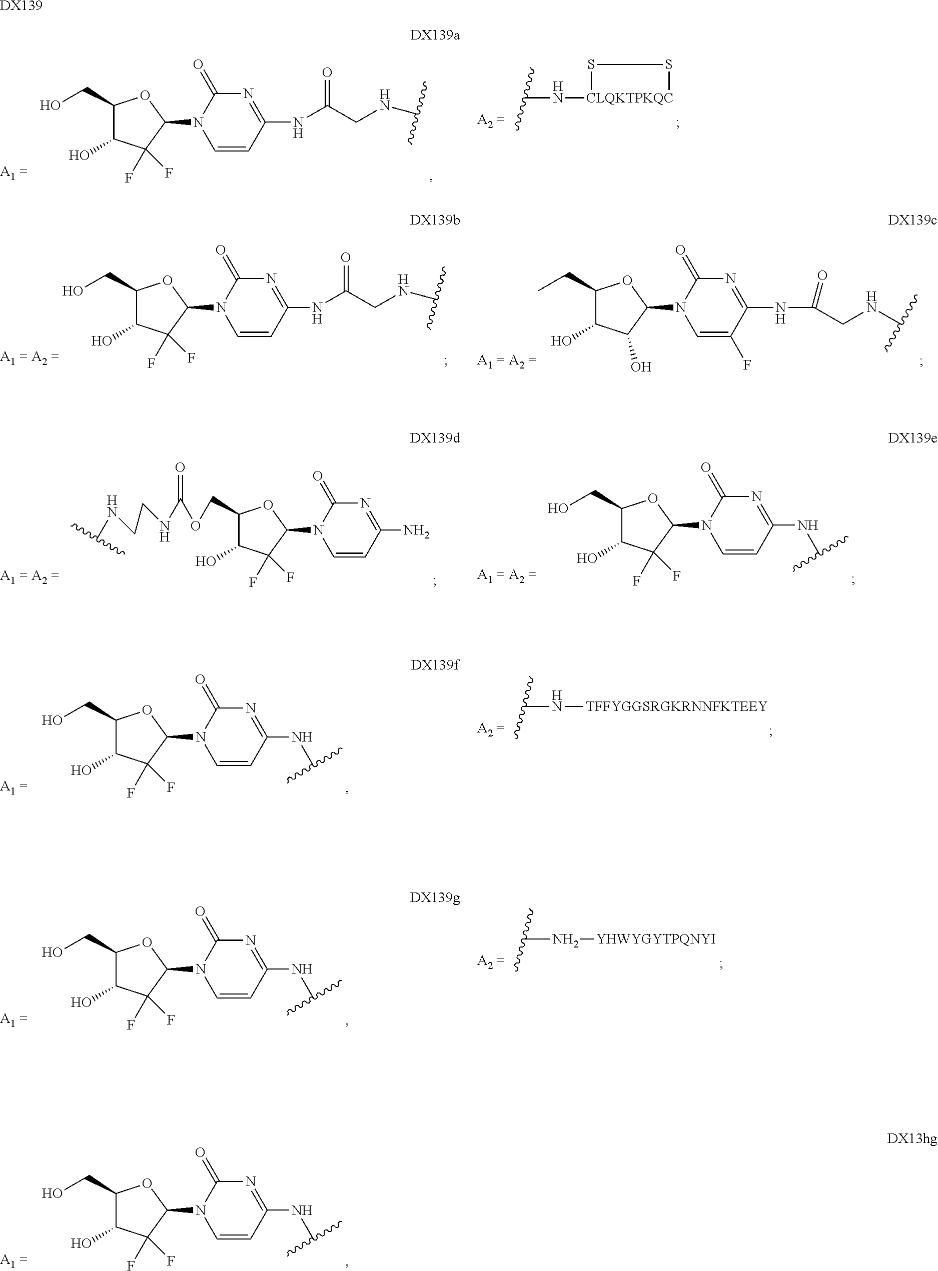

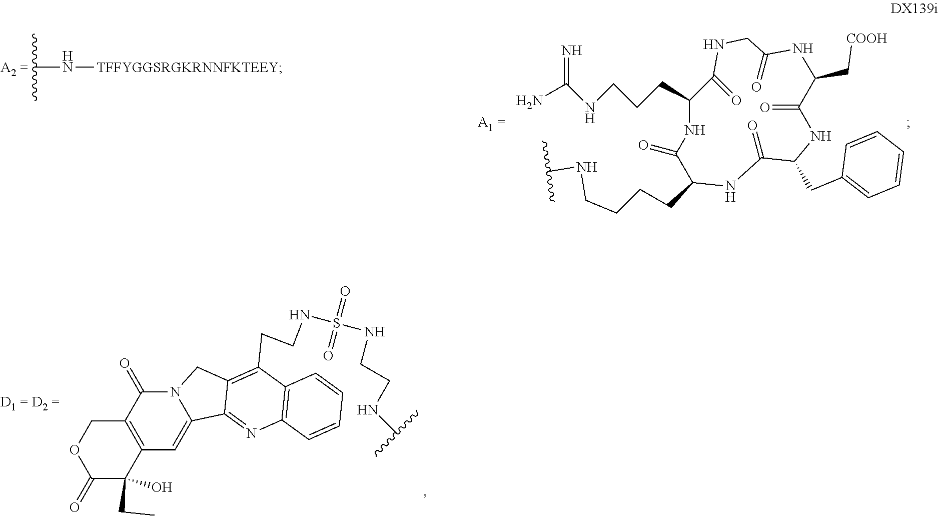

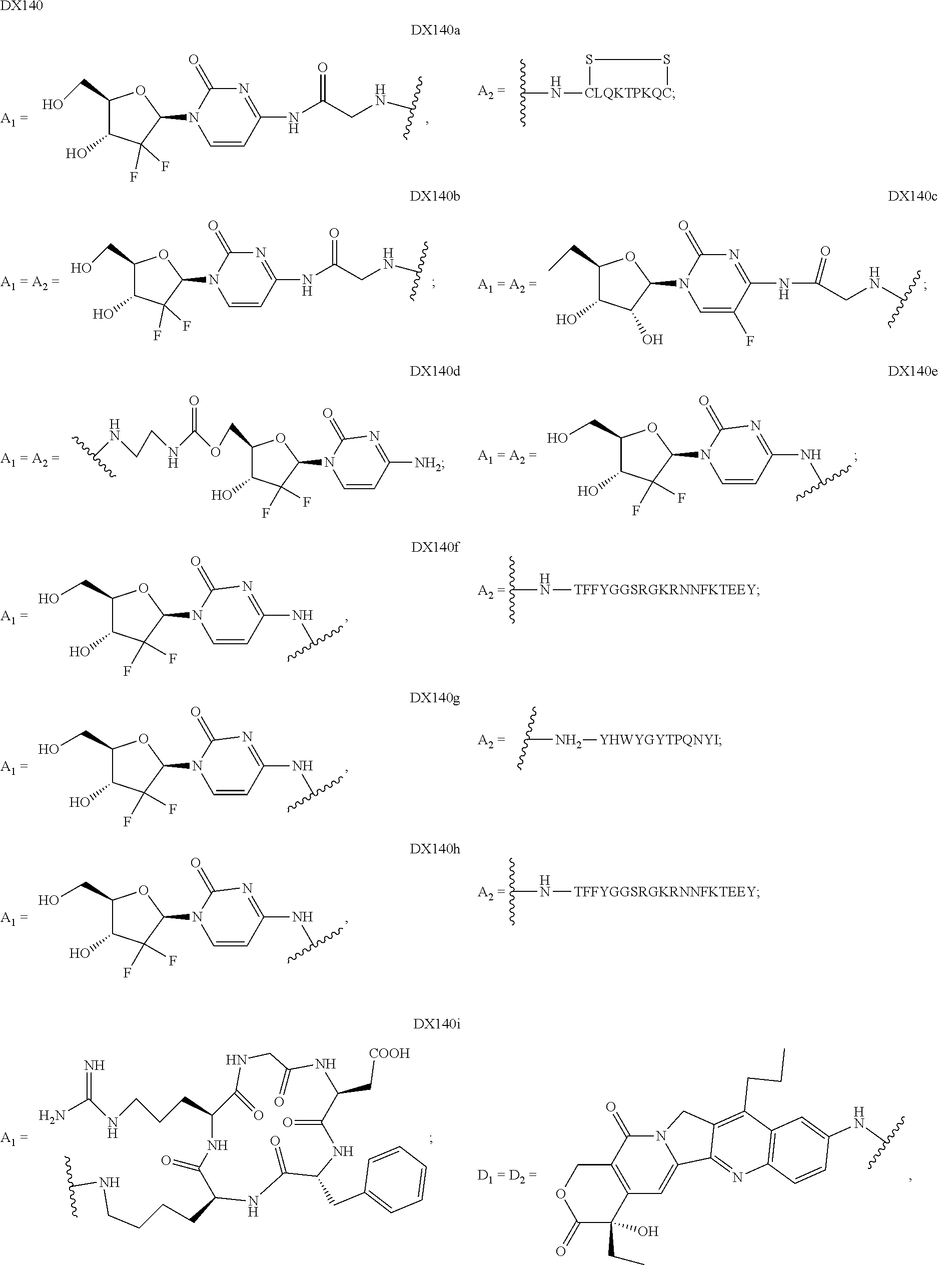

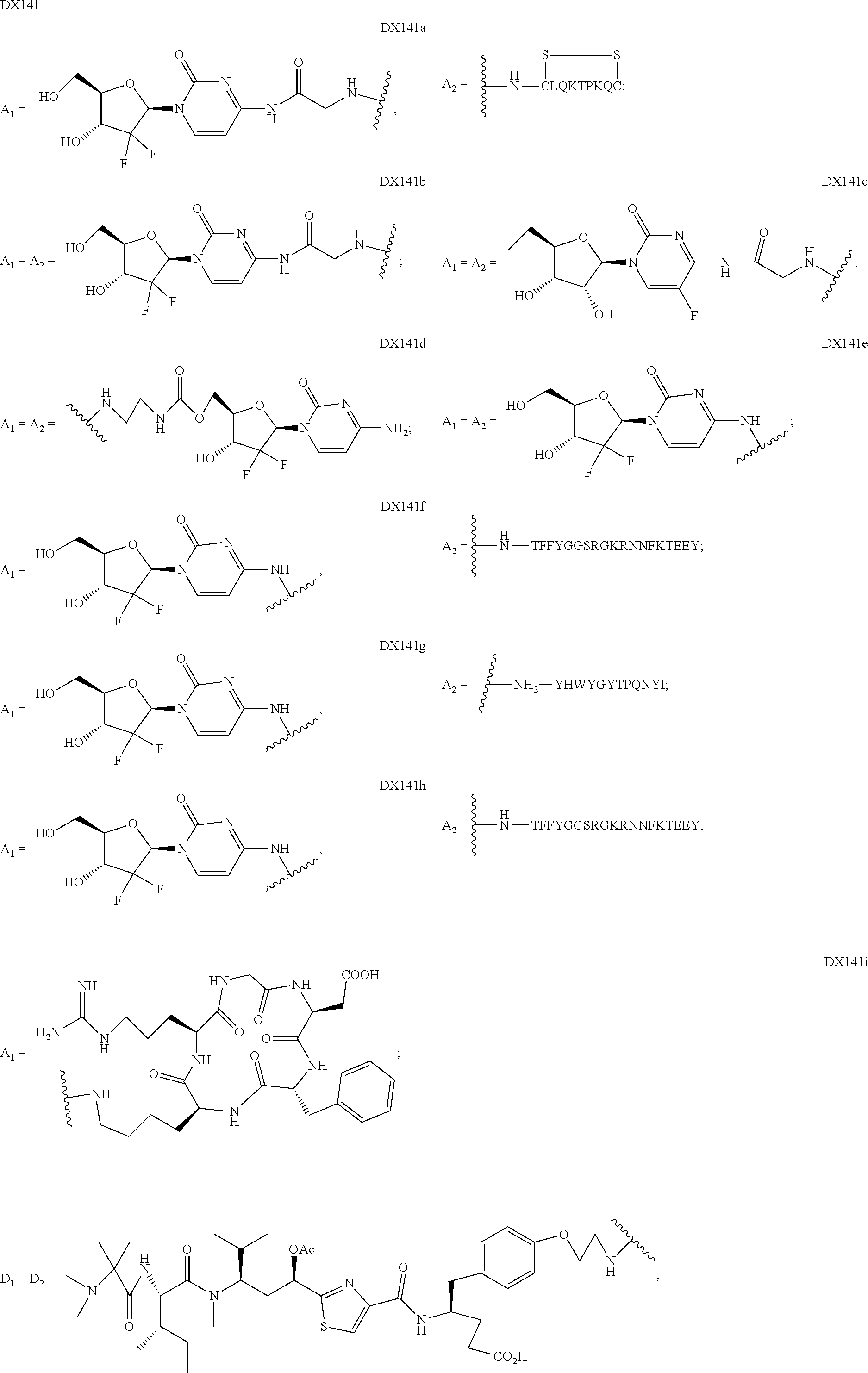

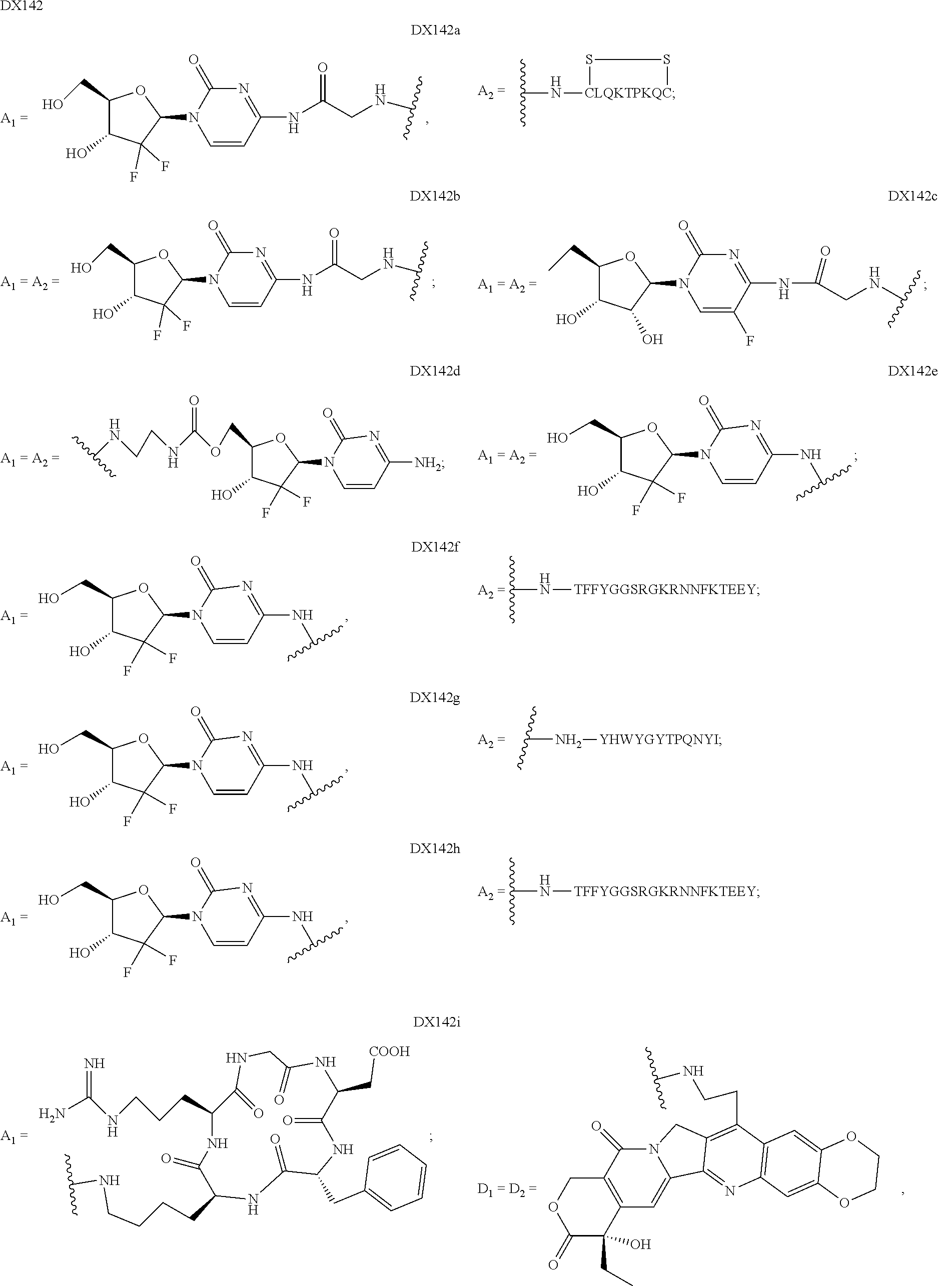

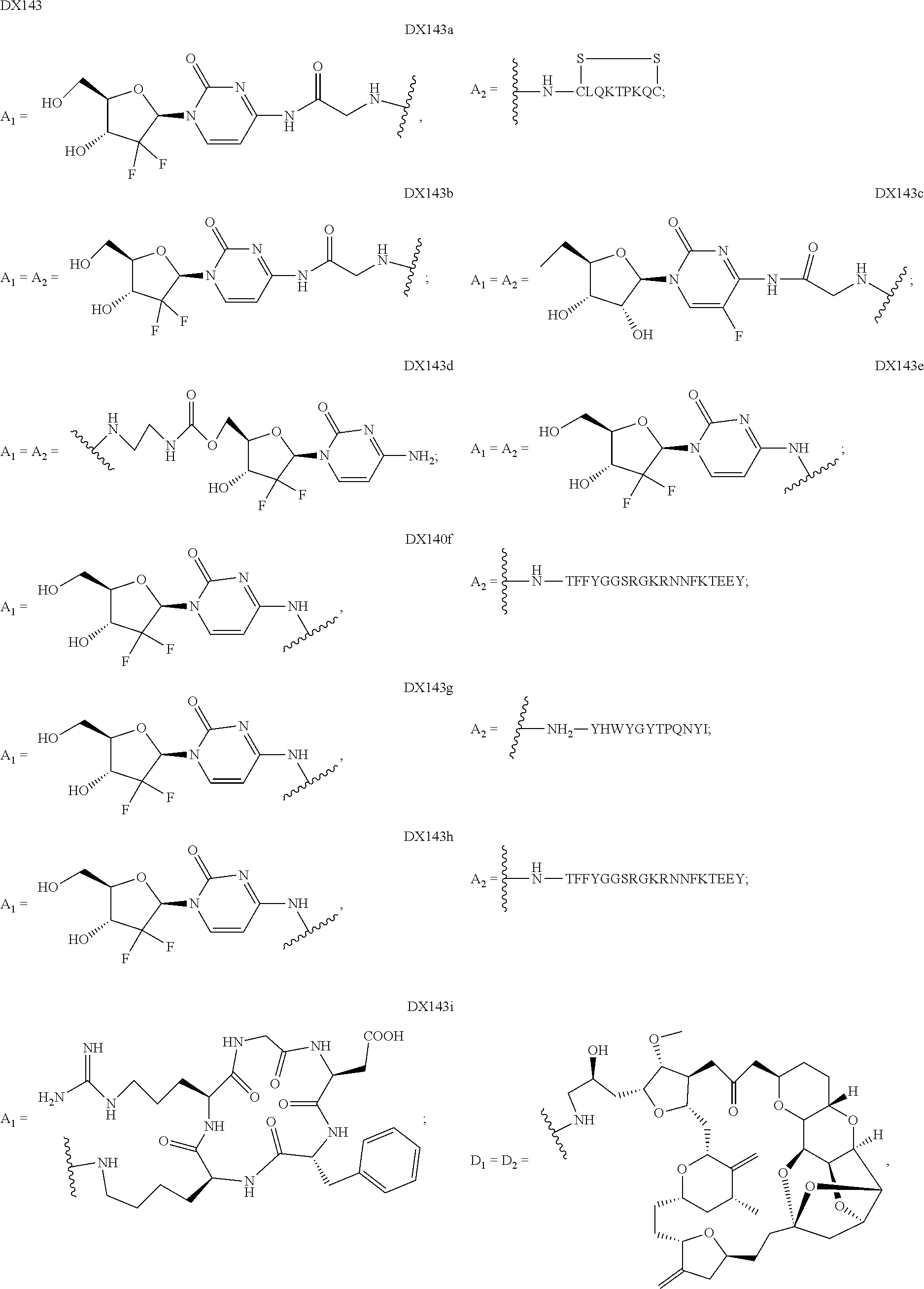

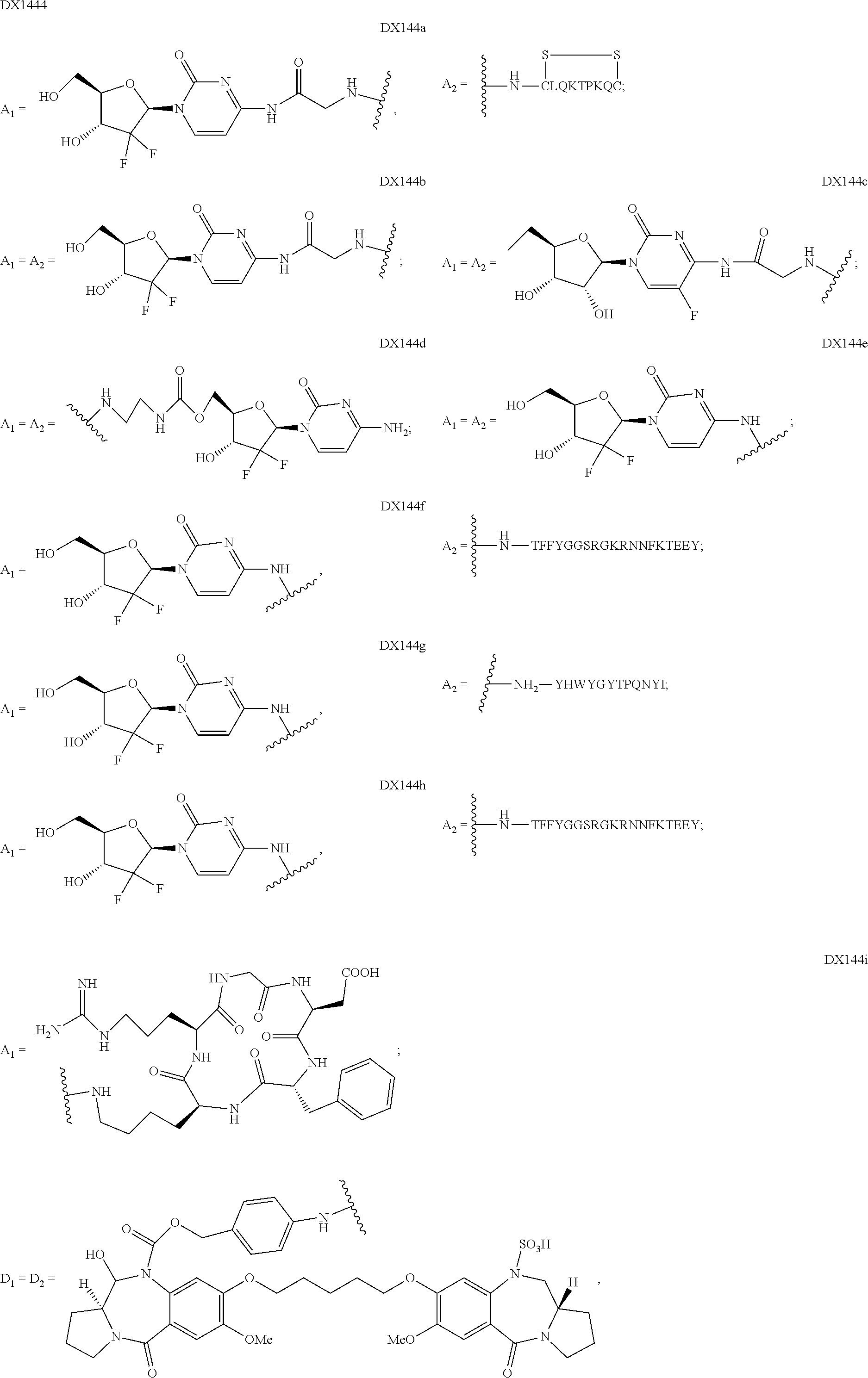

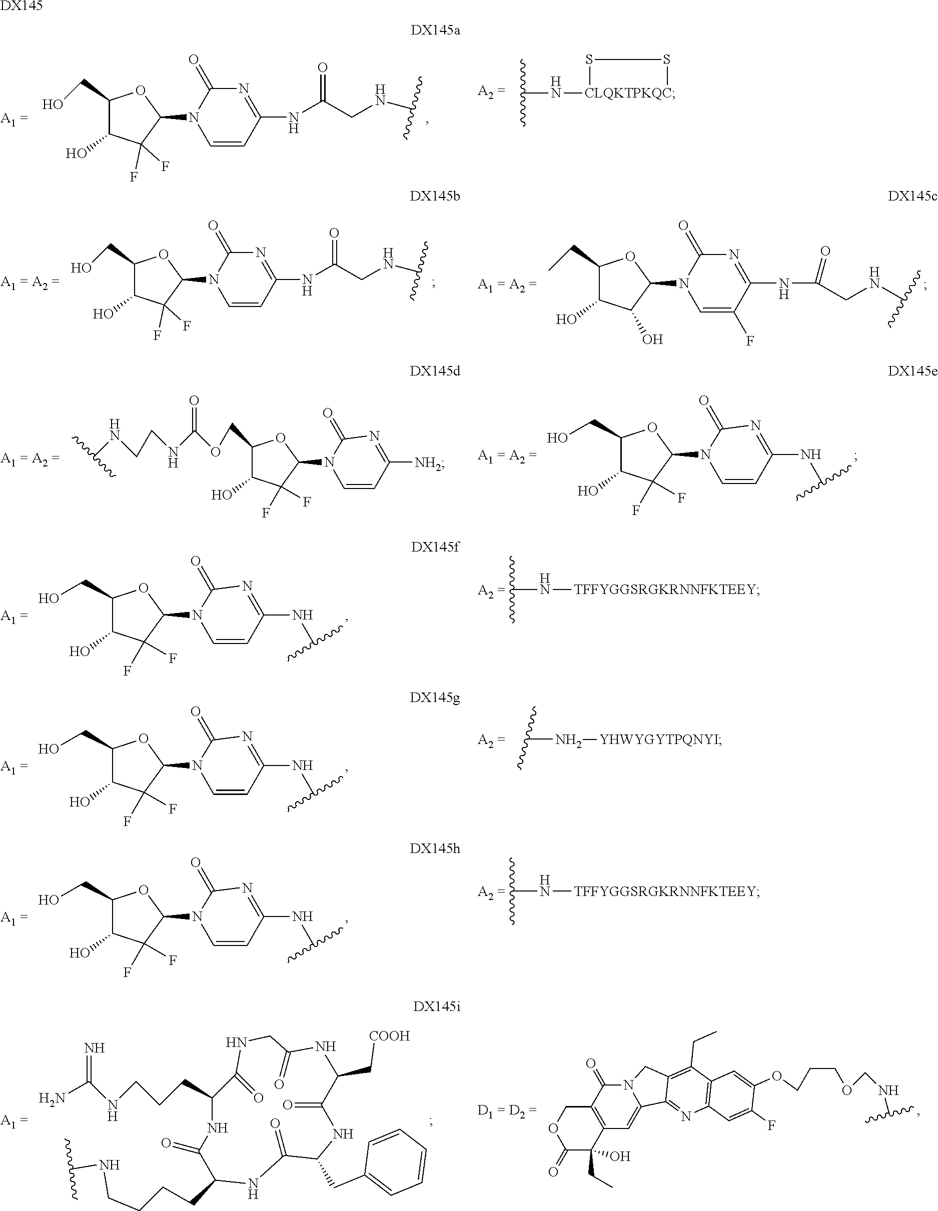

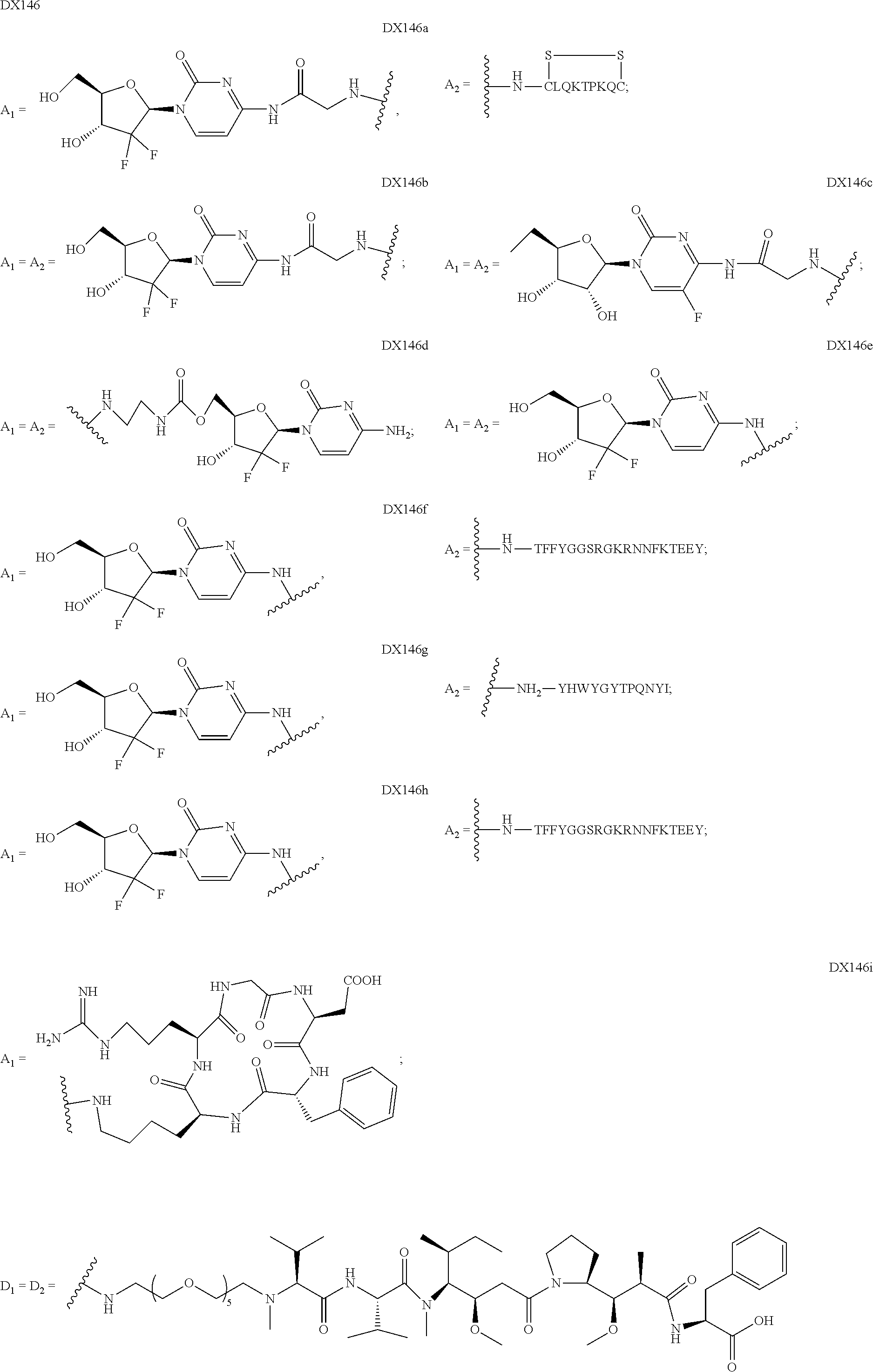

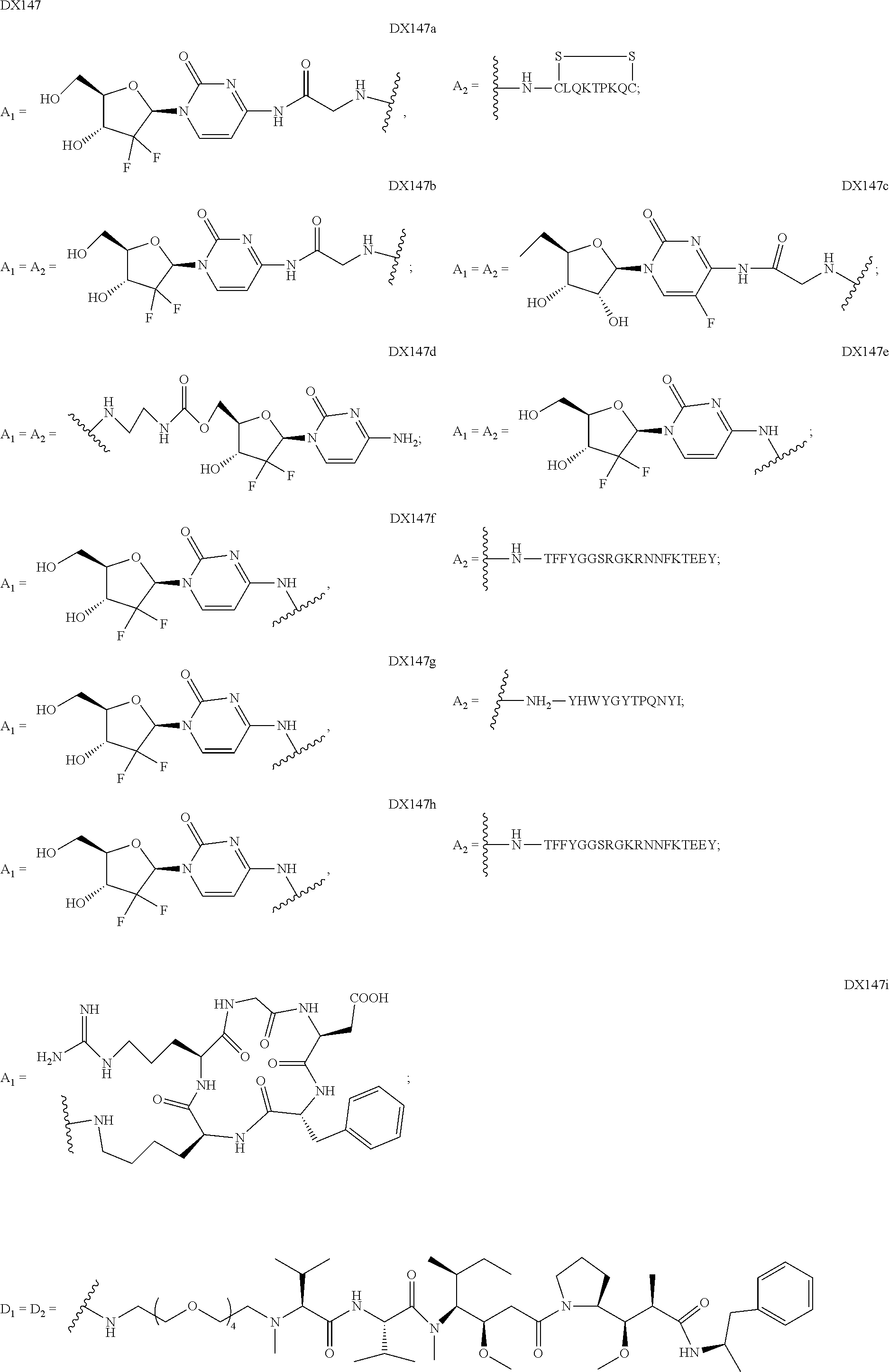

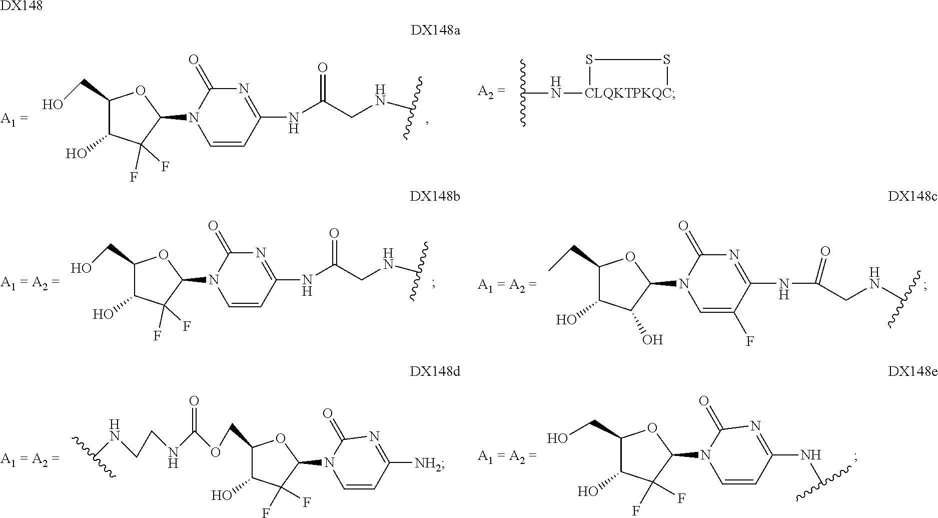

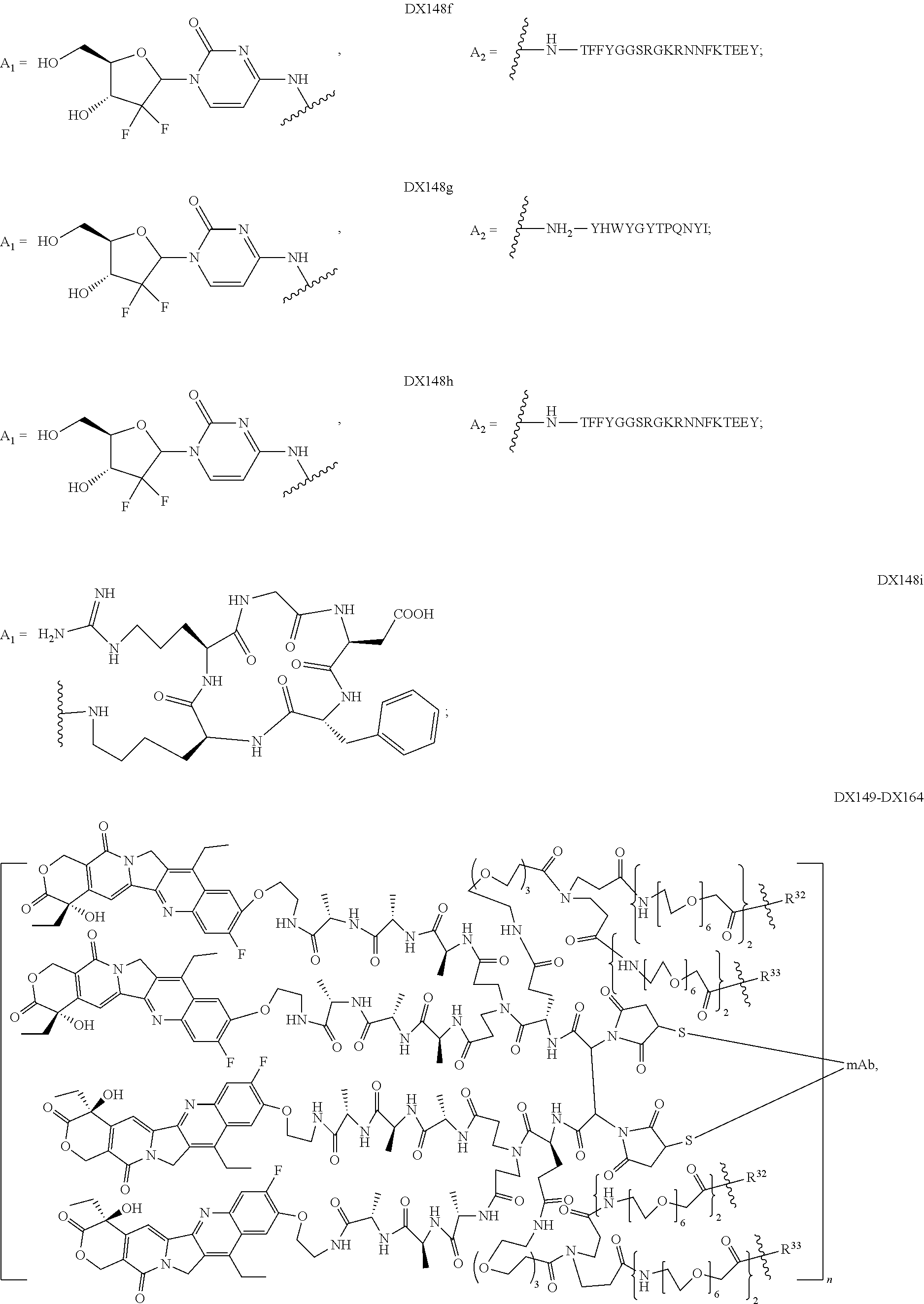

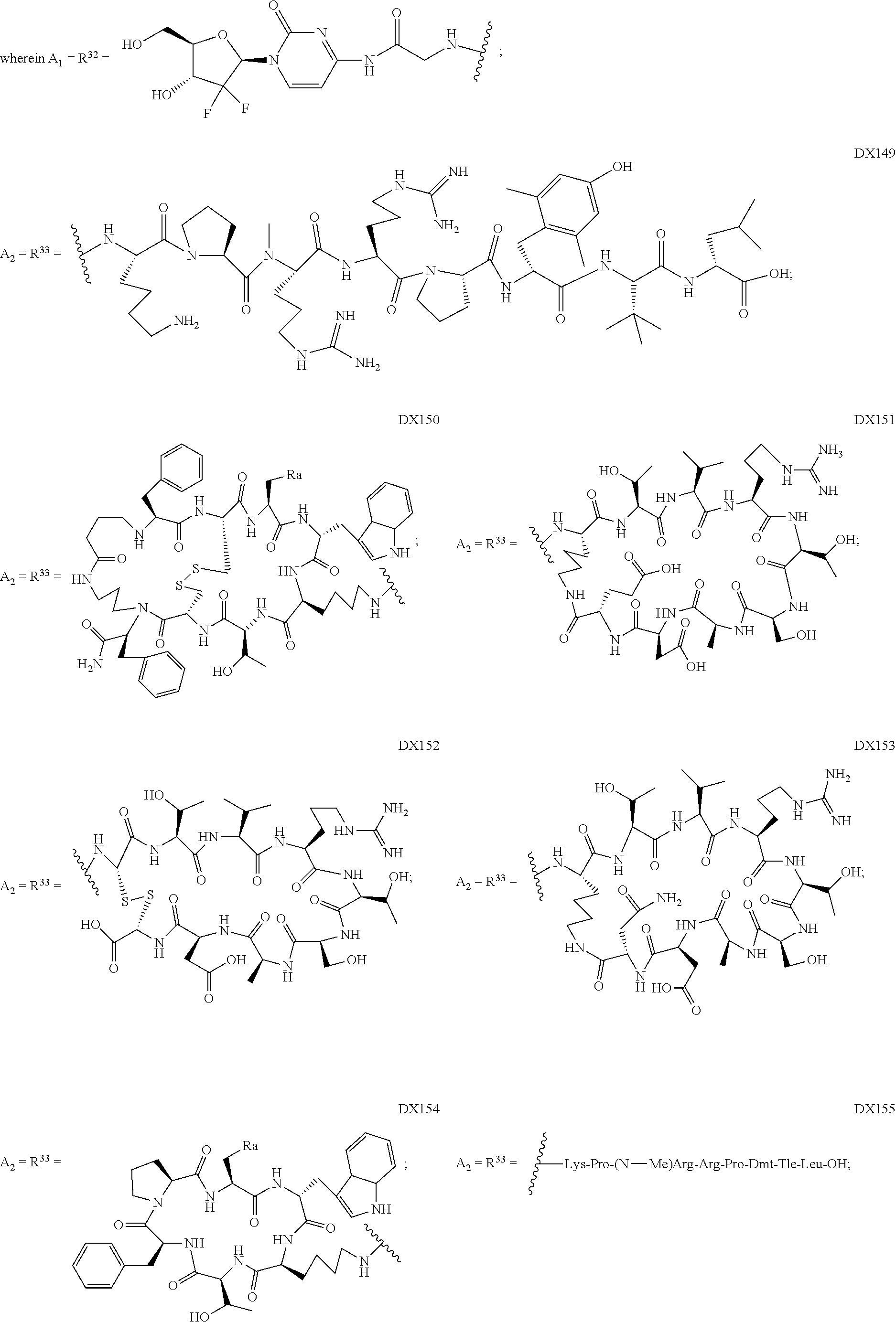

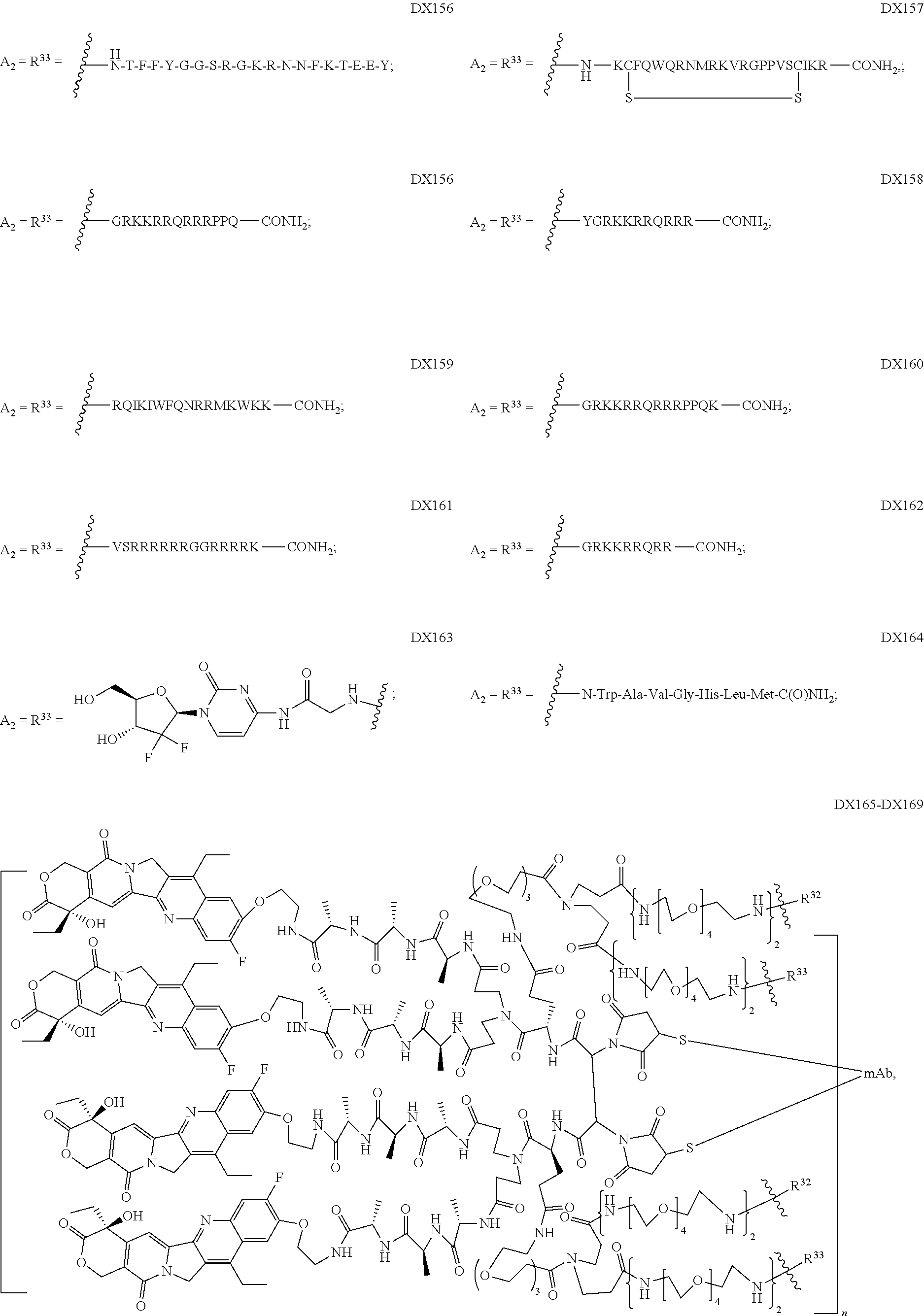

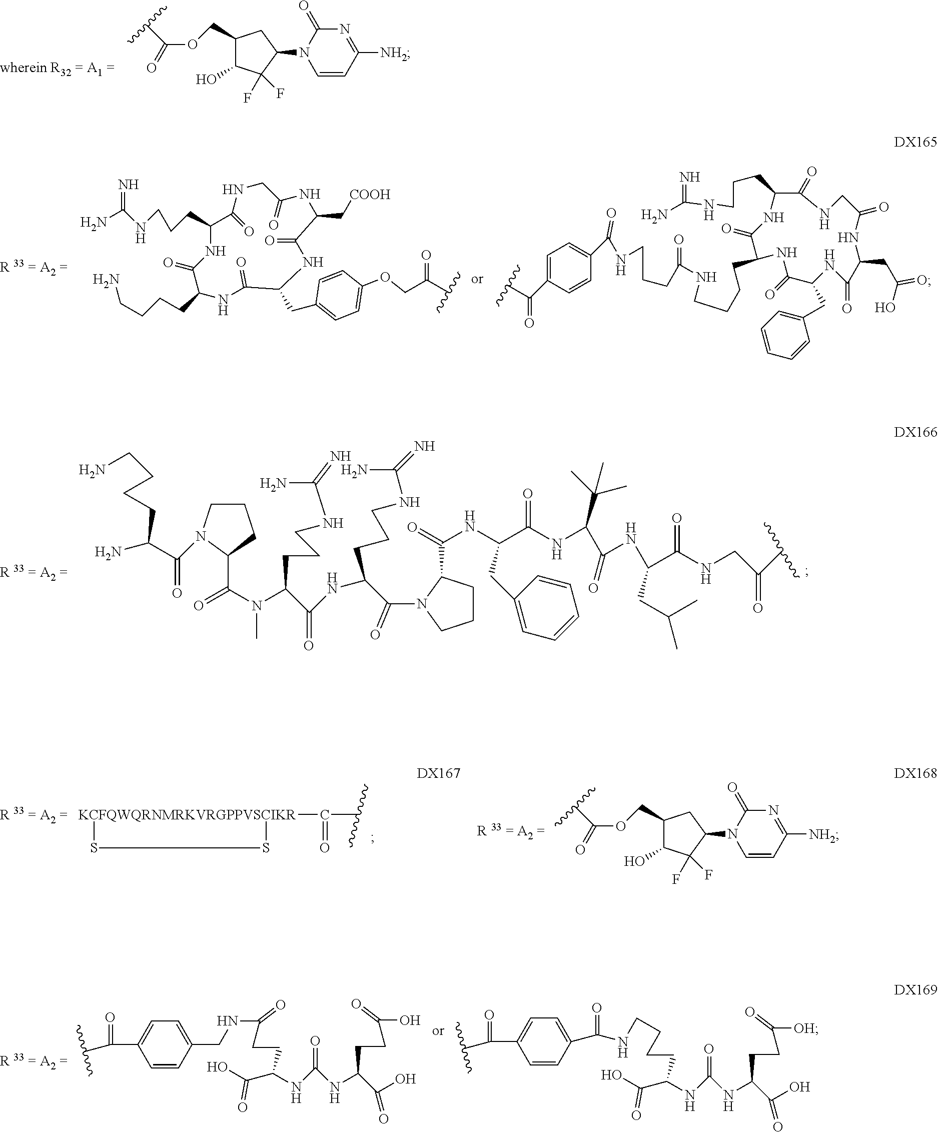

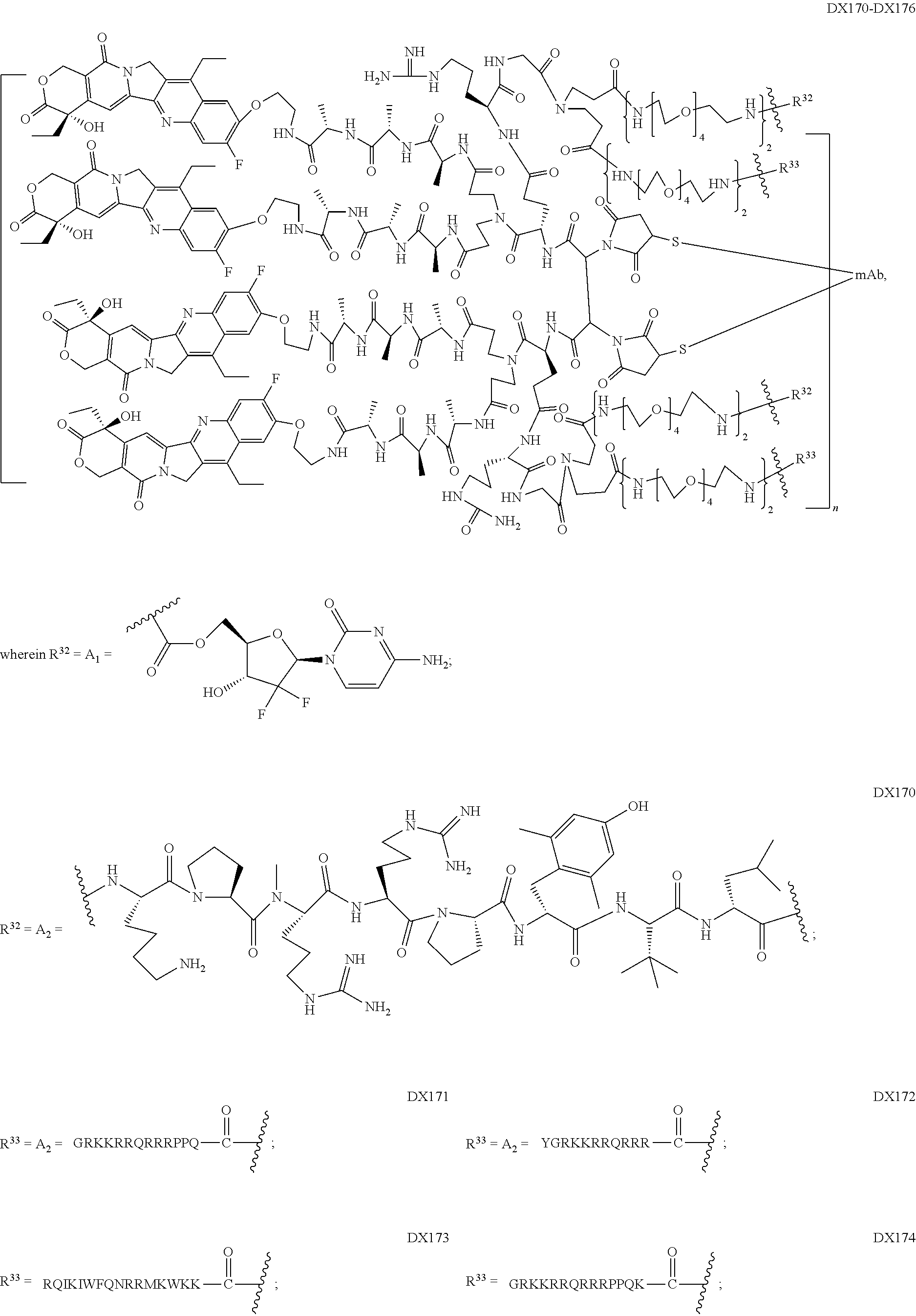

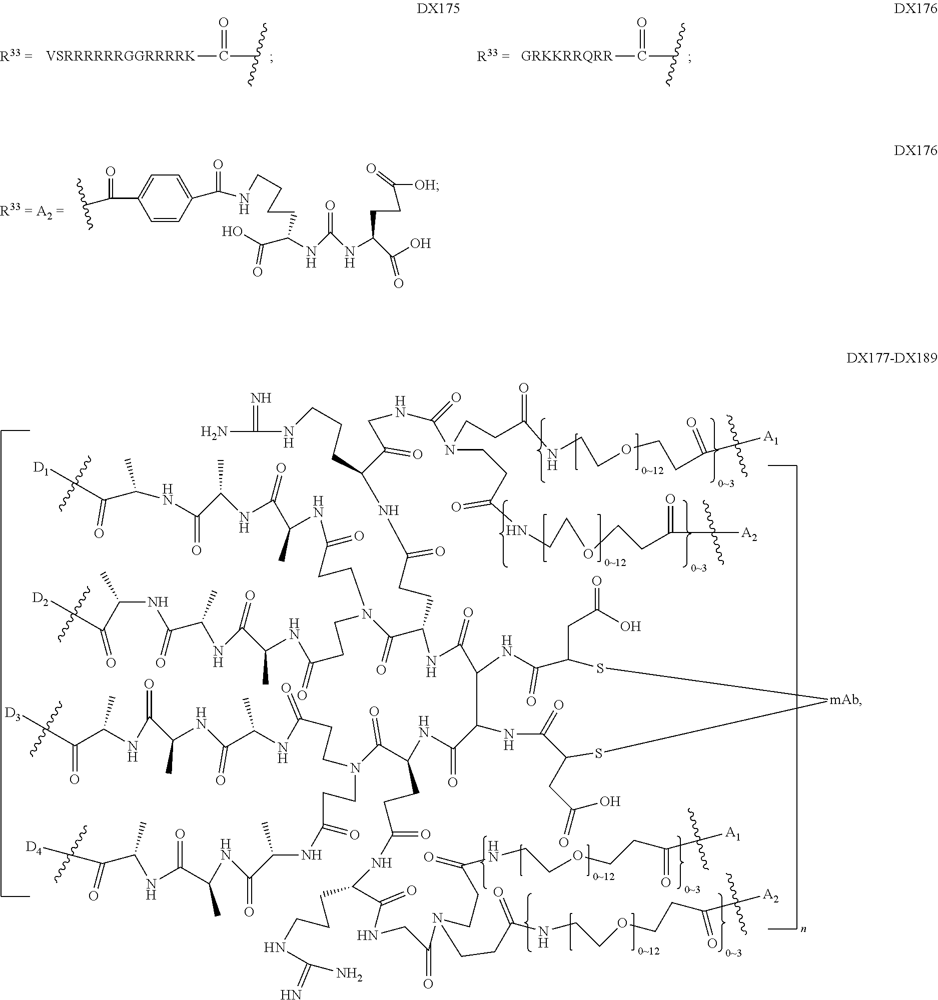

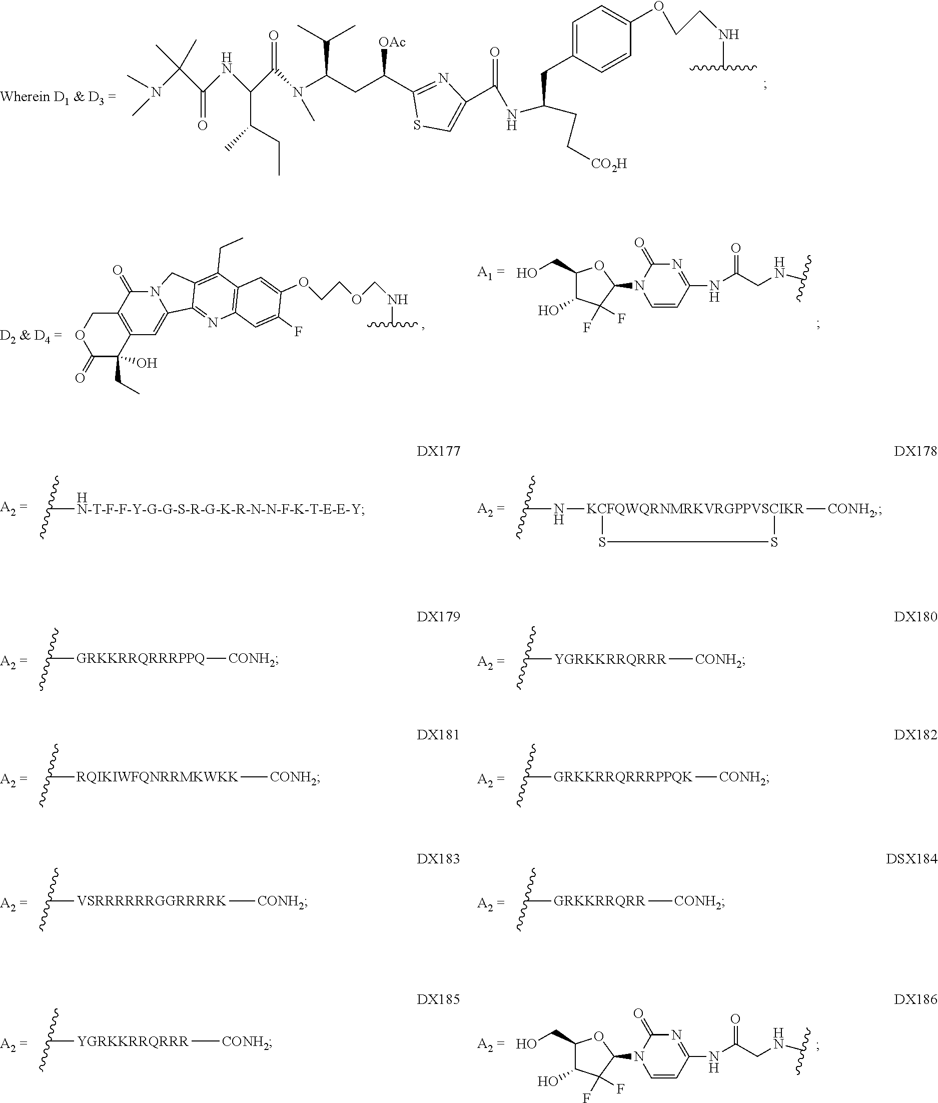

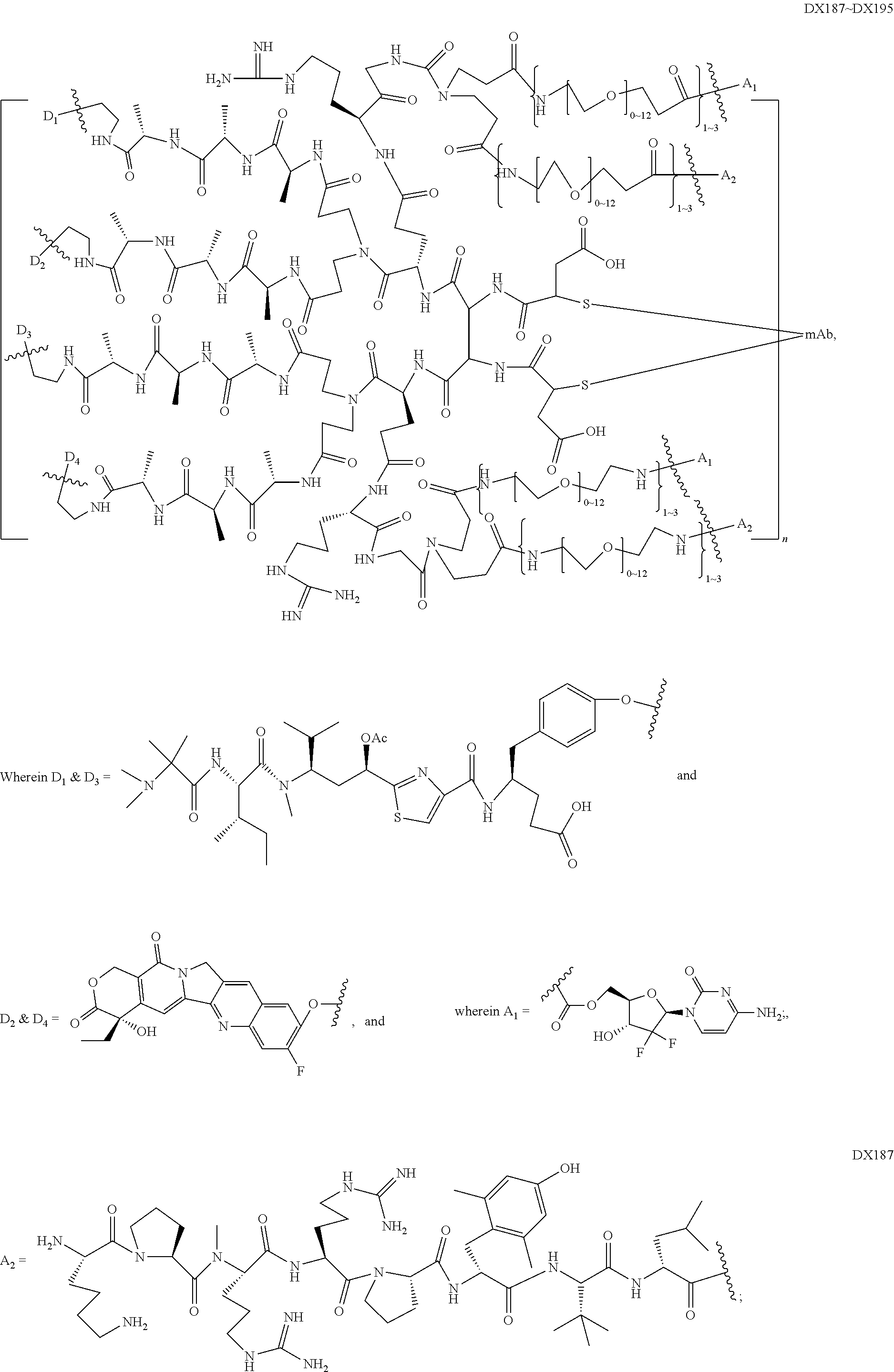

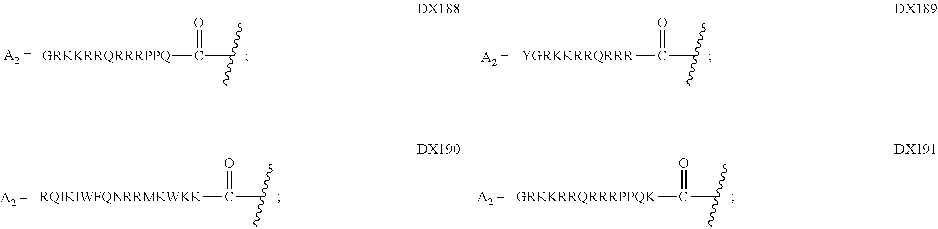

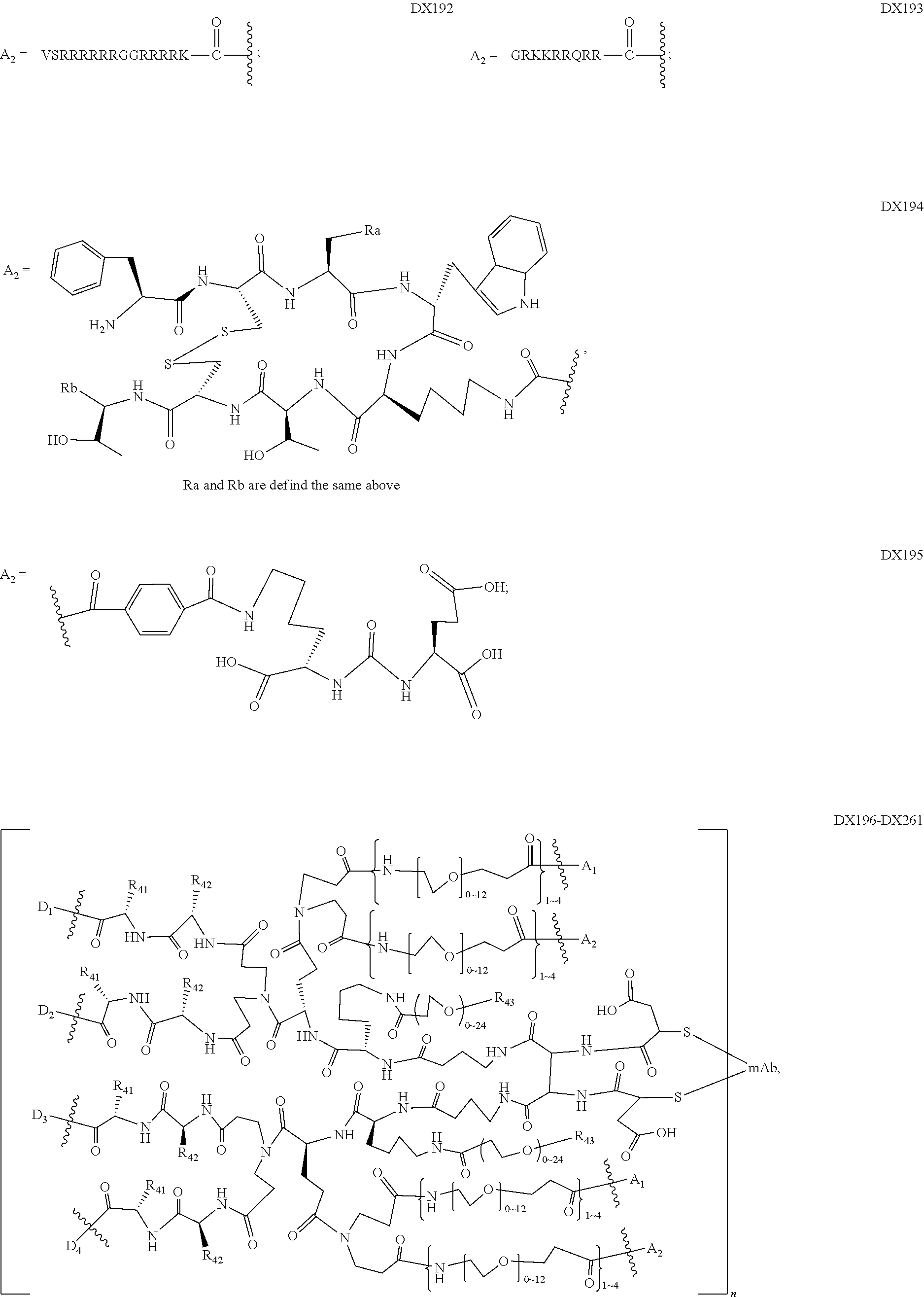

Wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(—NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H₄OH; R₄₃ is H, CH₃, CH₂CH₃, C(O)H, and C(O)CH₃; D1, D₂, D3 and D4 are cytotoxic drug described in this application, and A₁ and A₂ are small molecular ligands or drugs described in this application. DX196-DX210, wherein R₄₁—CH₃, R₄₂=(CH₂)₄NHSO₂CH₃,

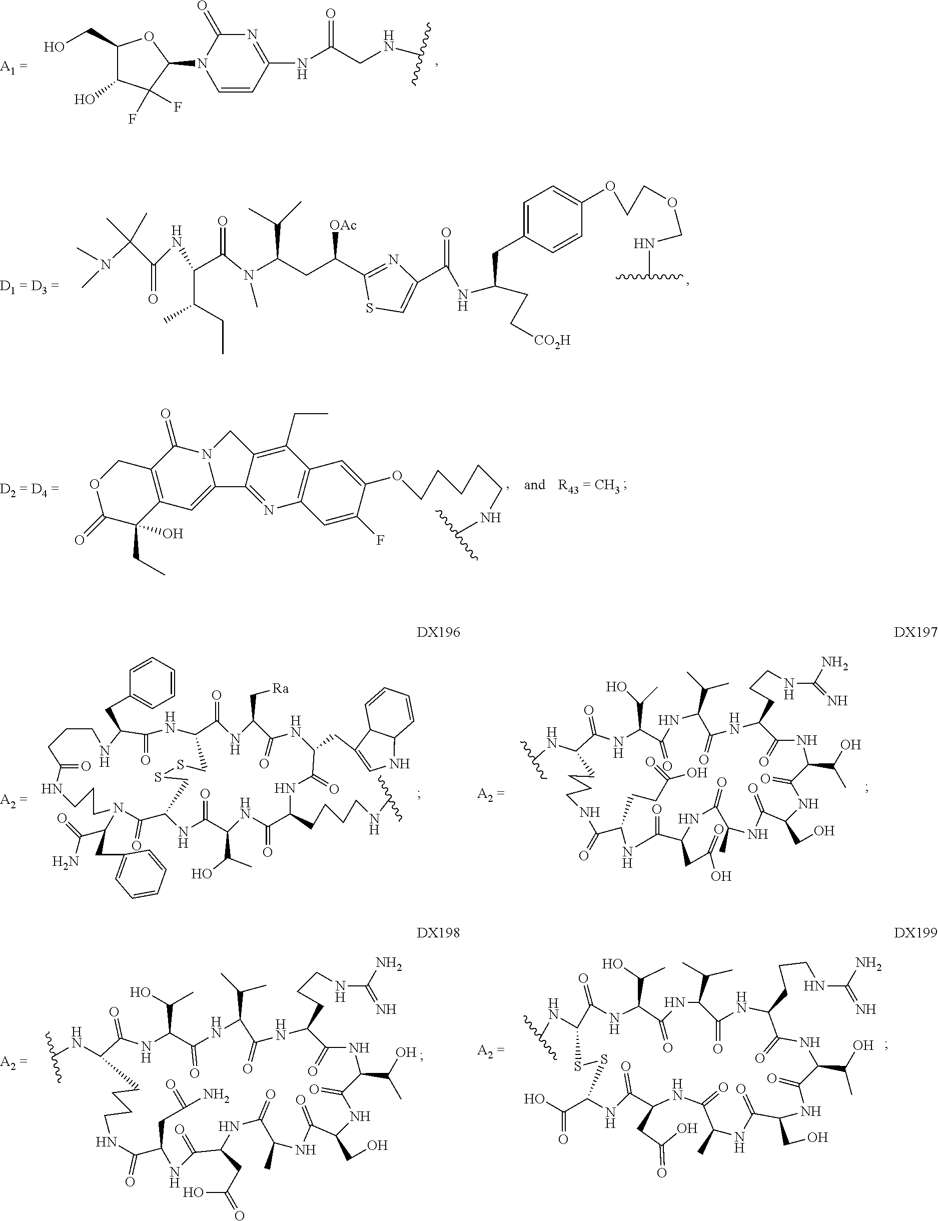

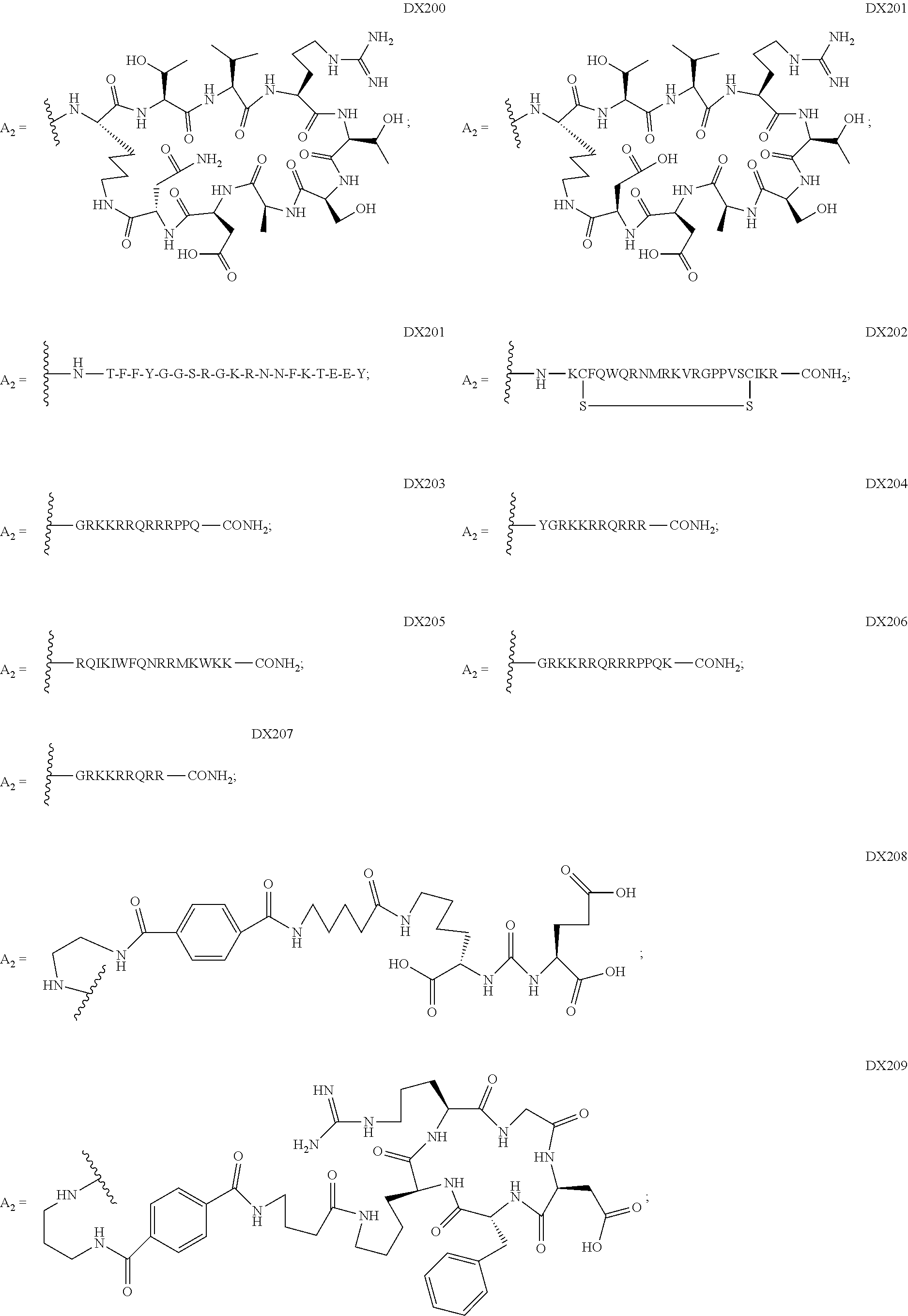

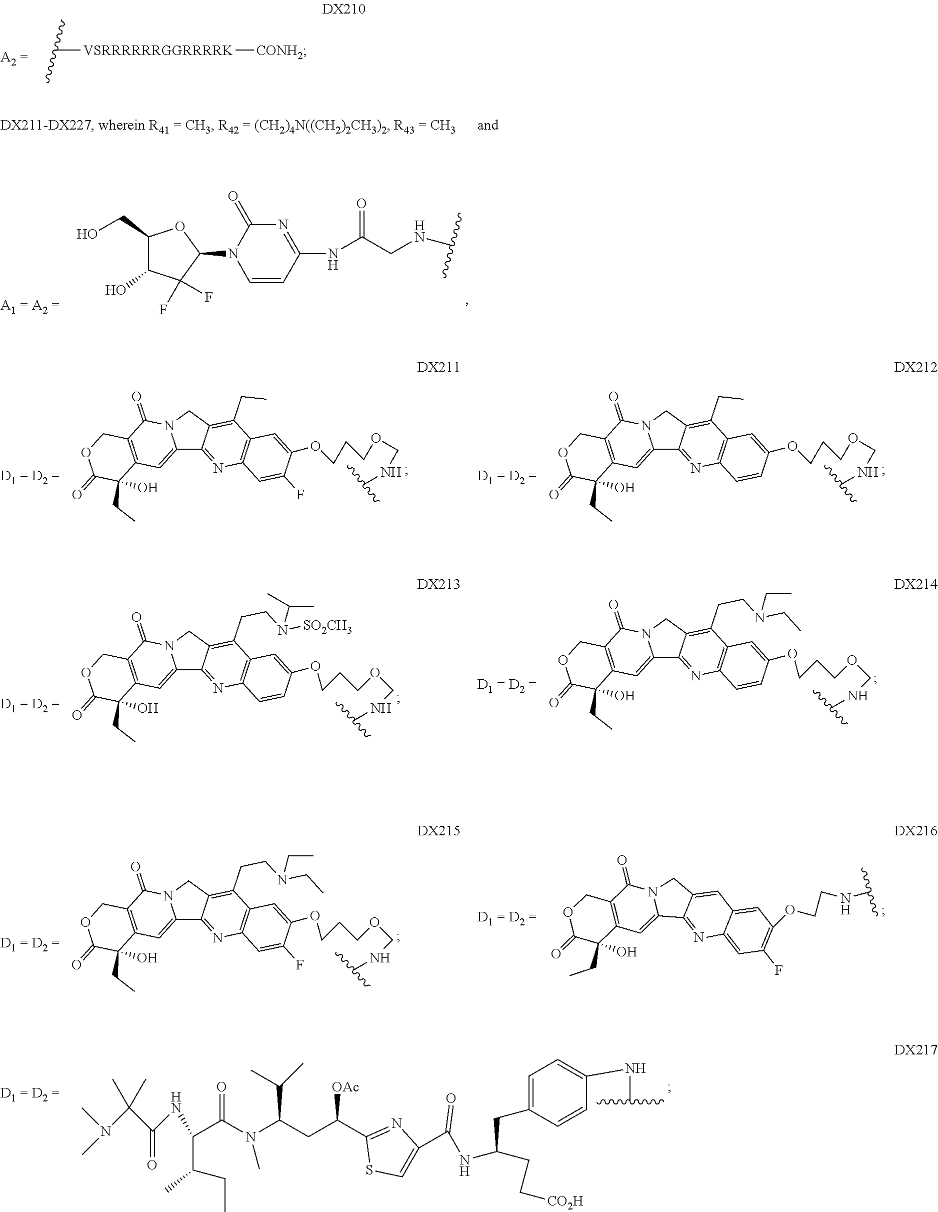

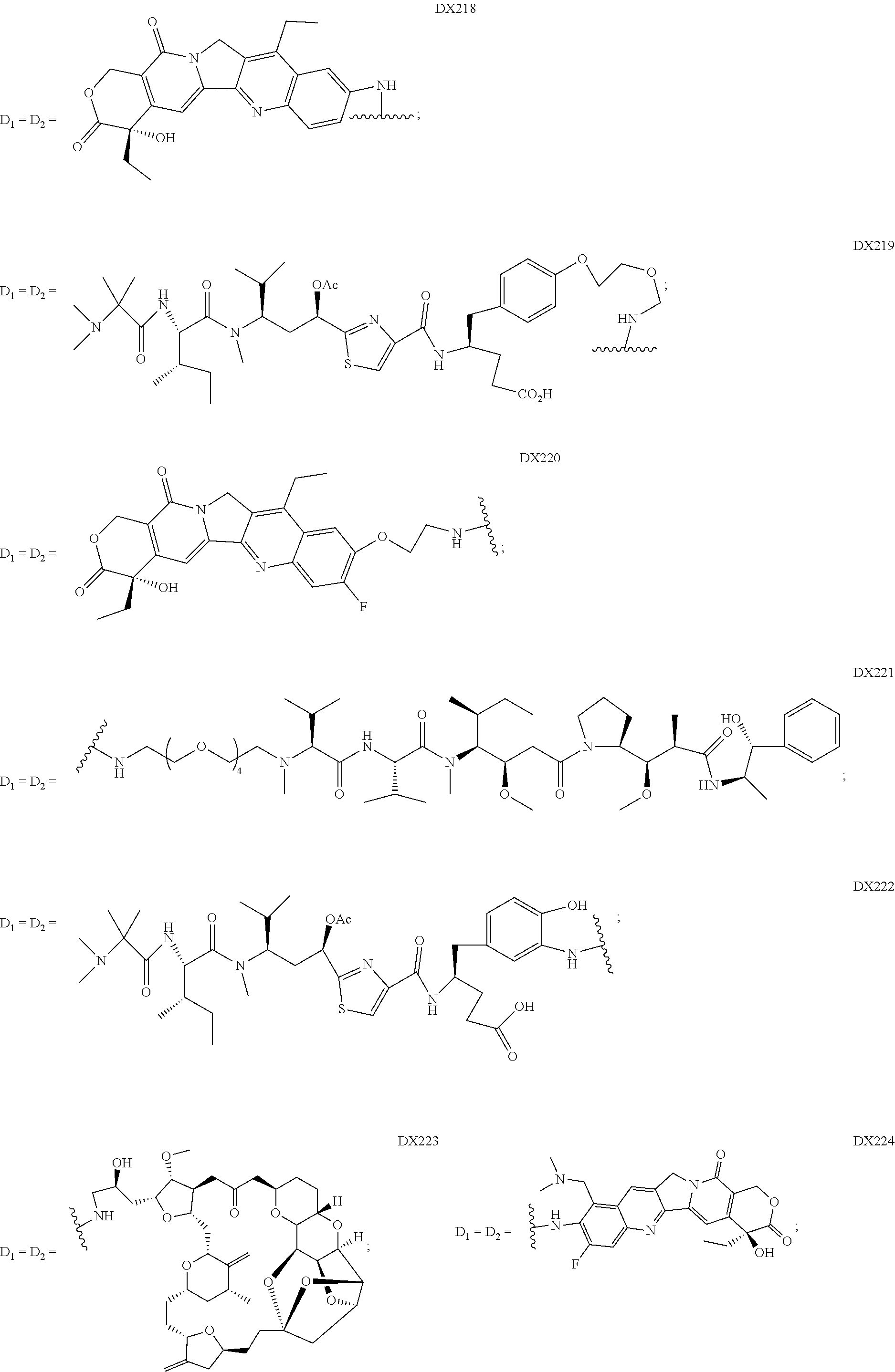

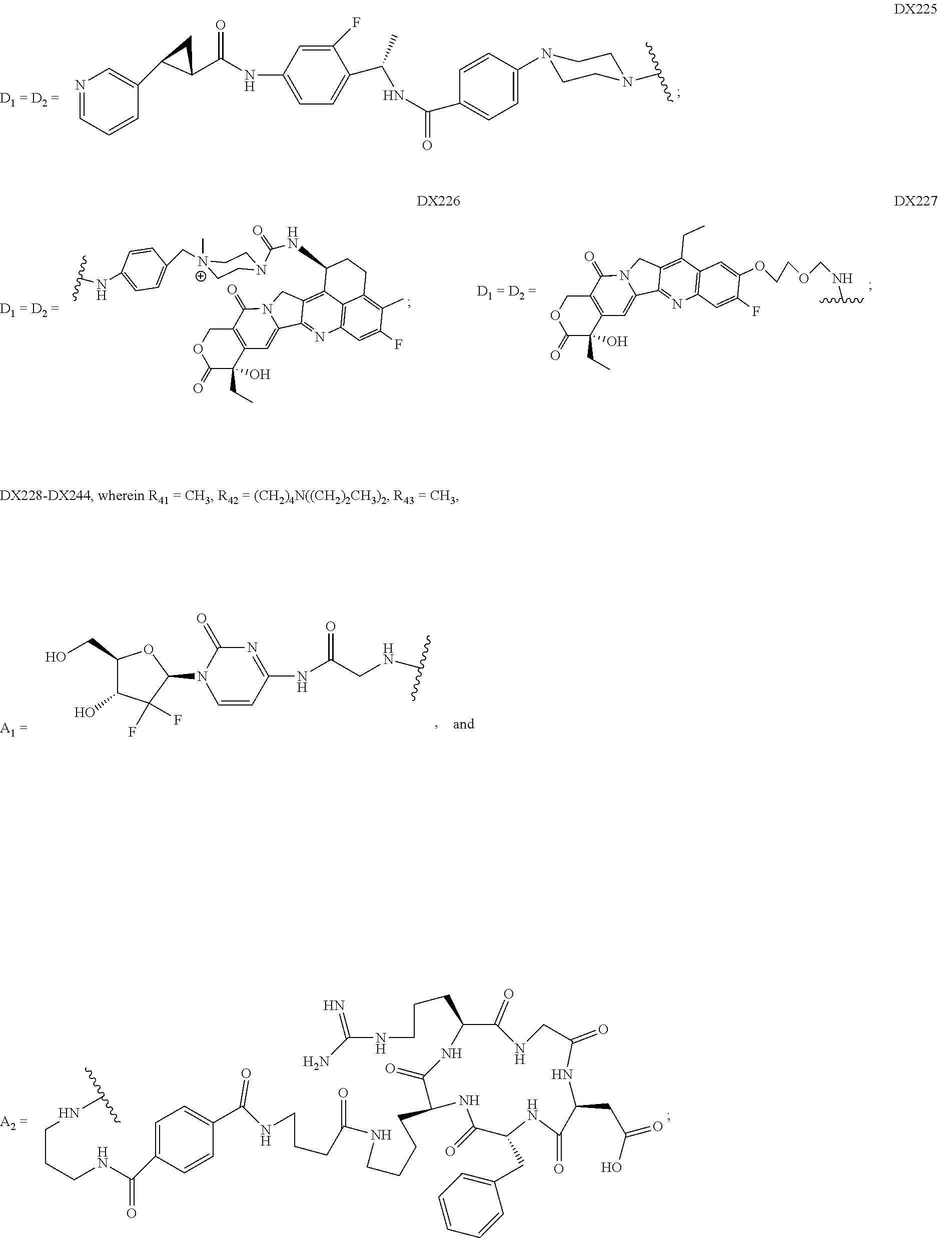

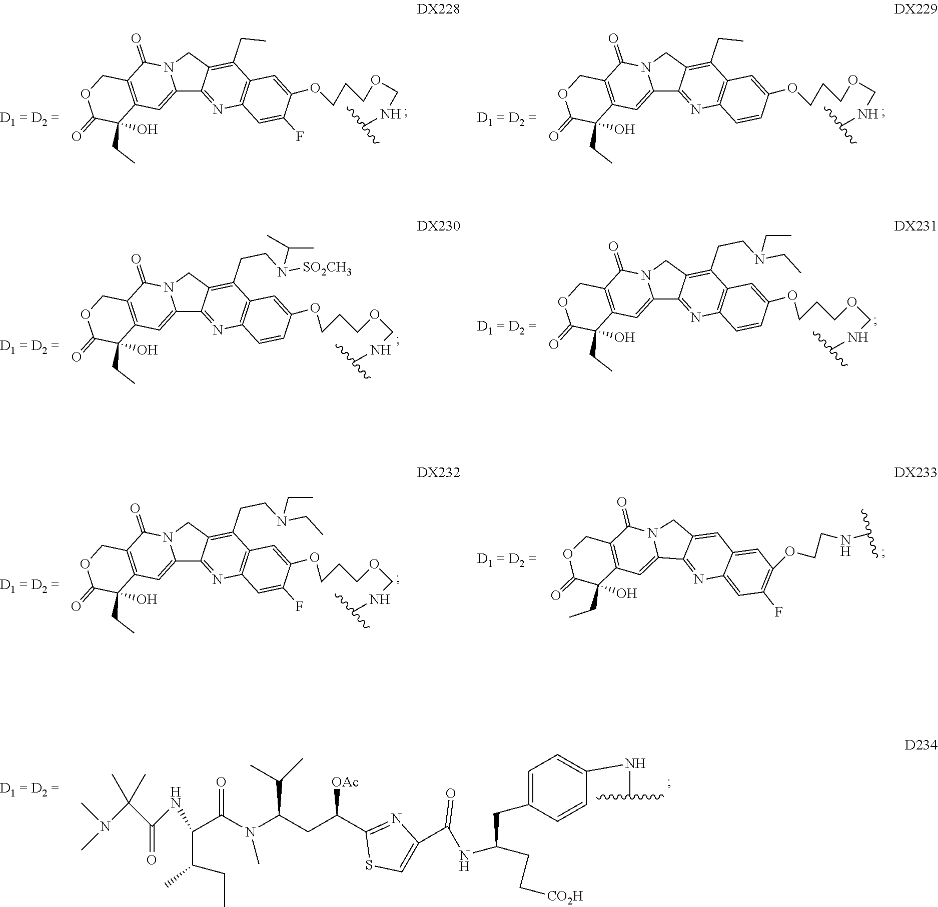

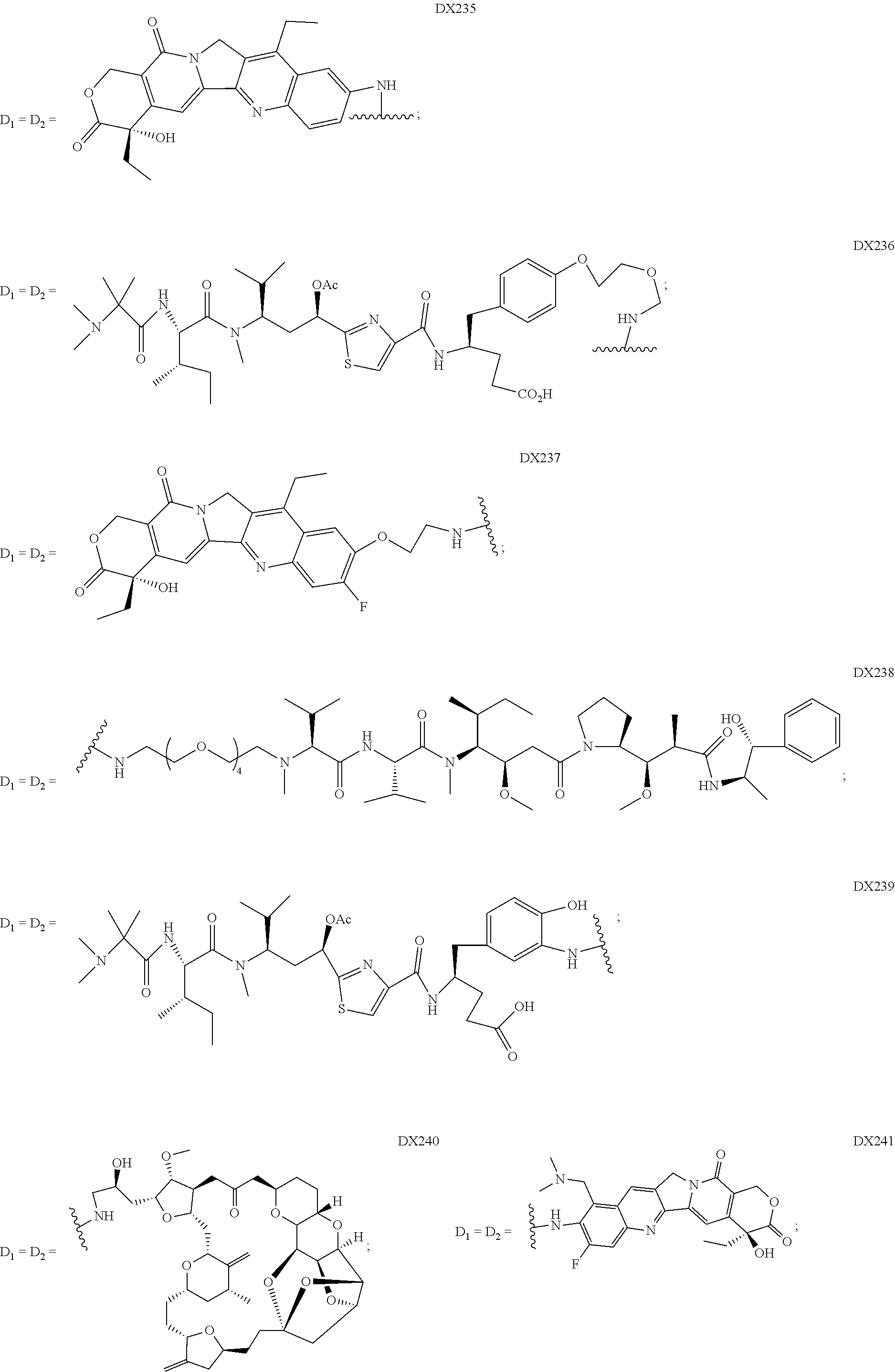

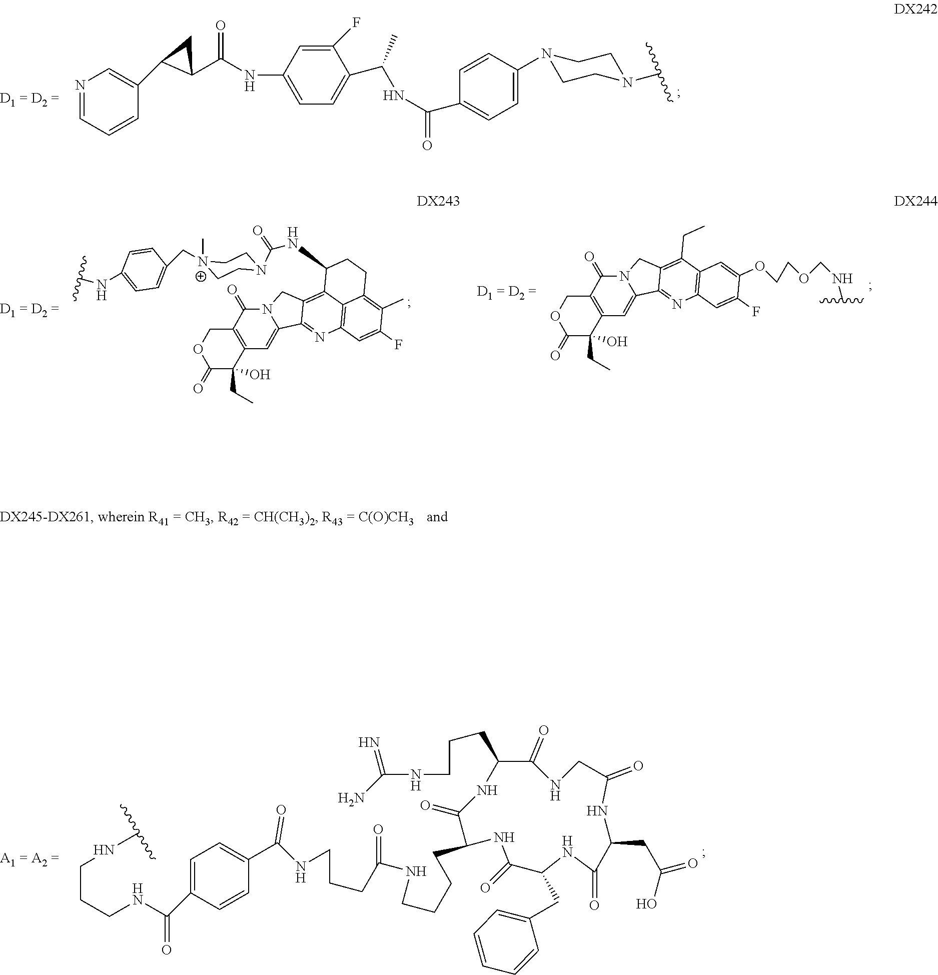

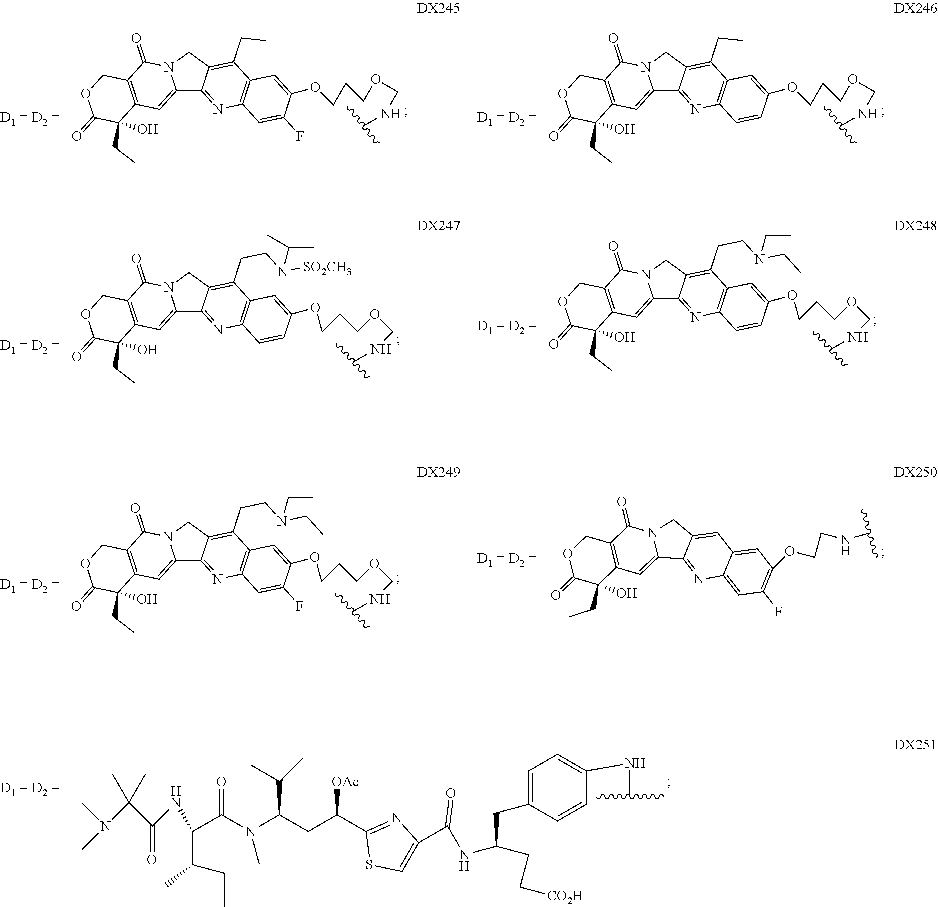

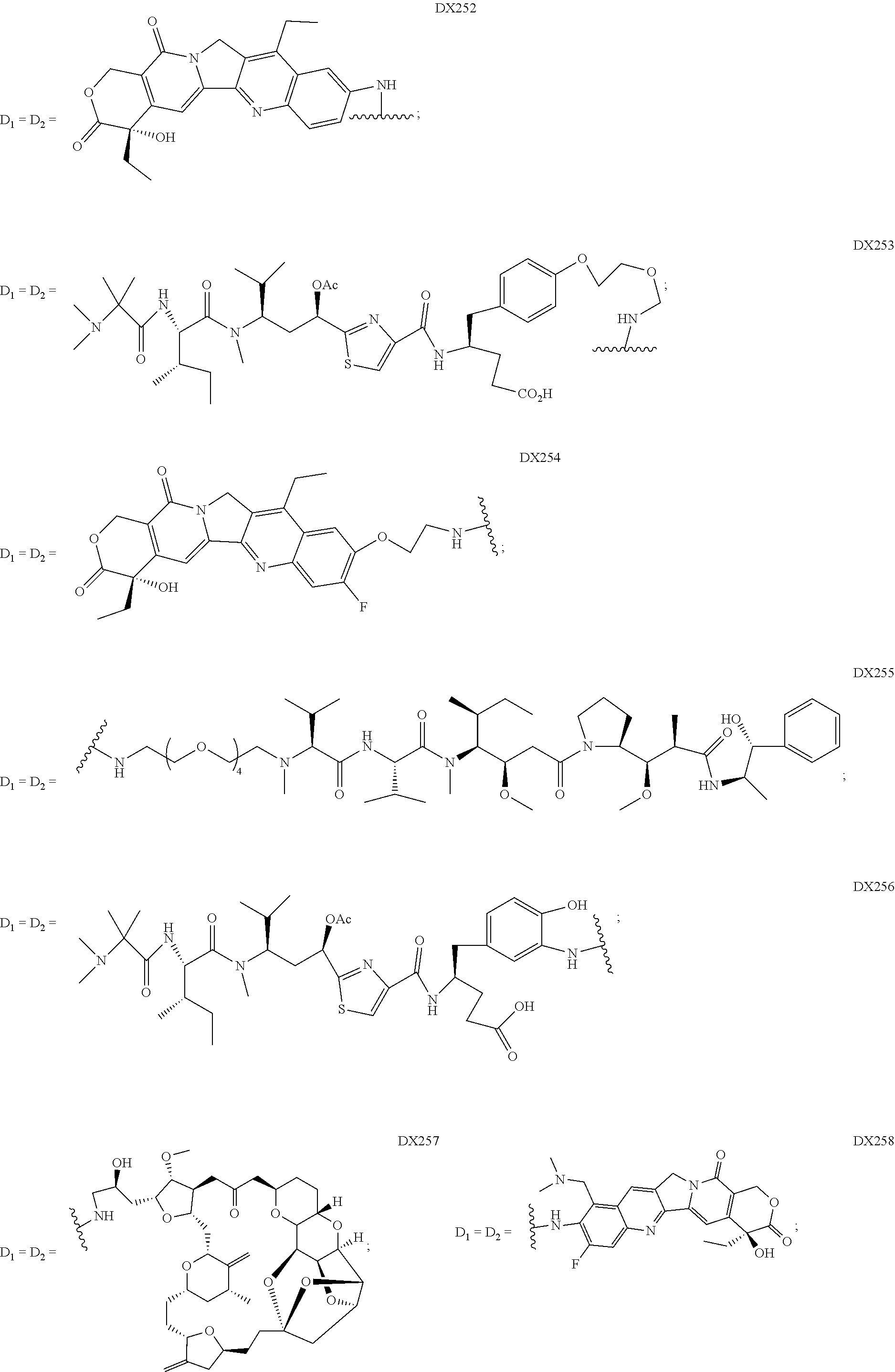

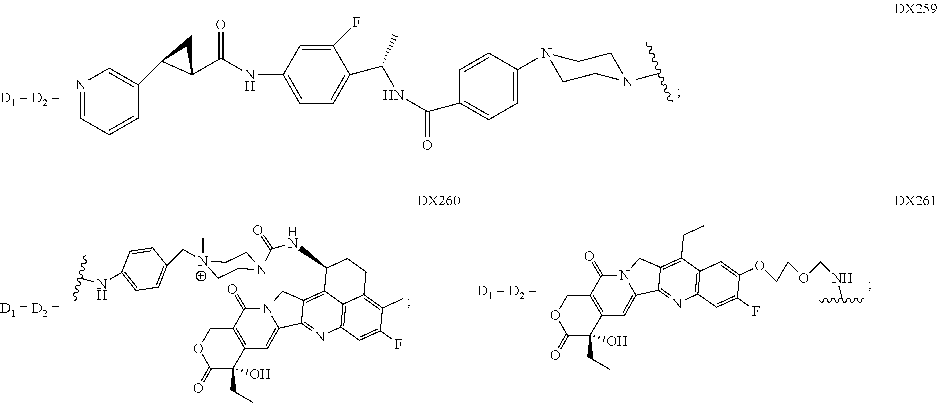

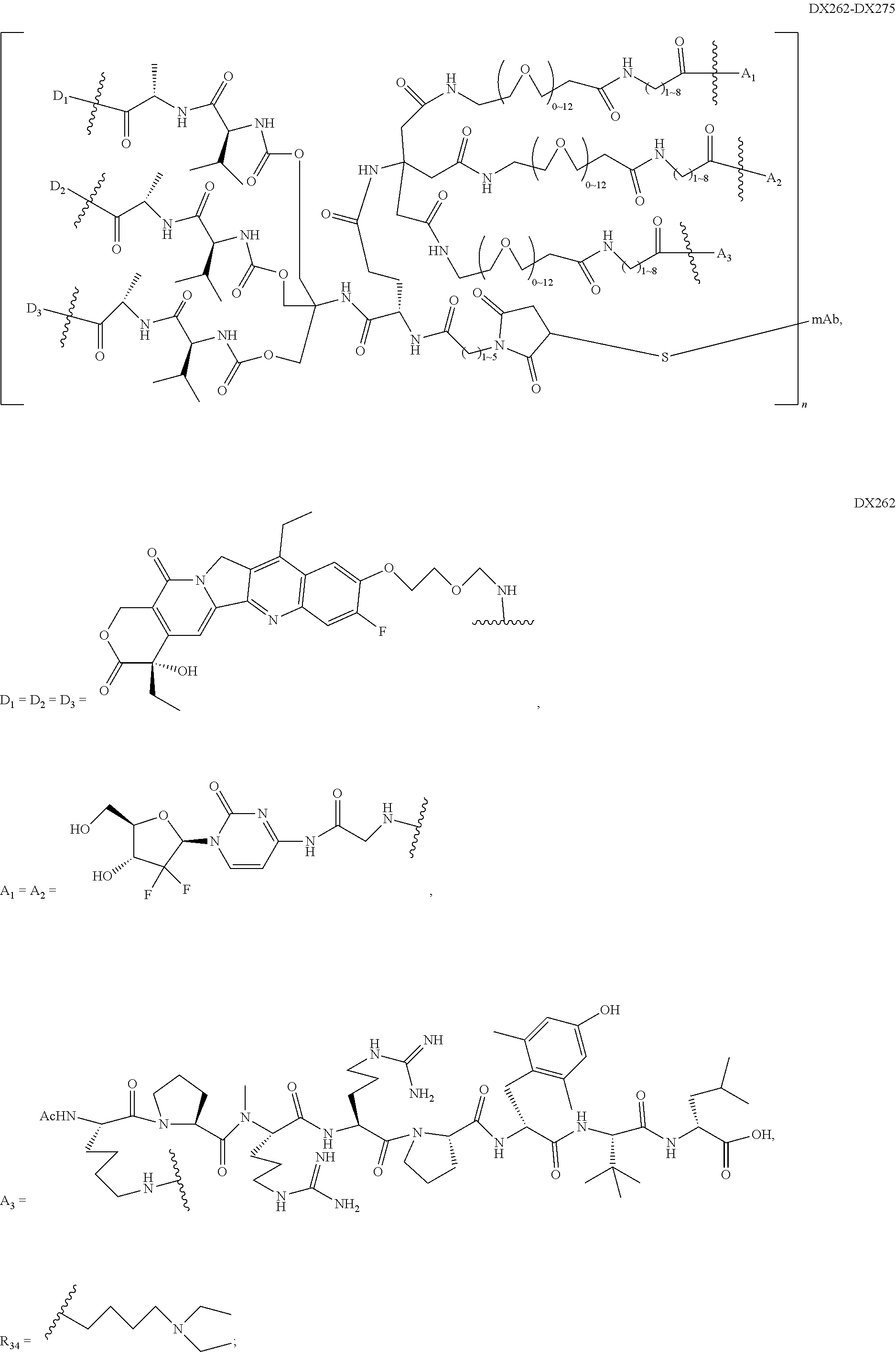

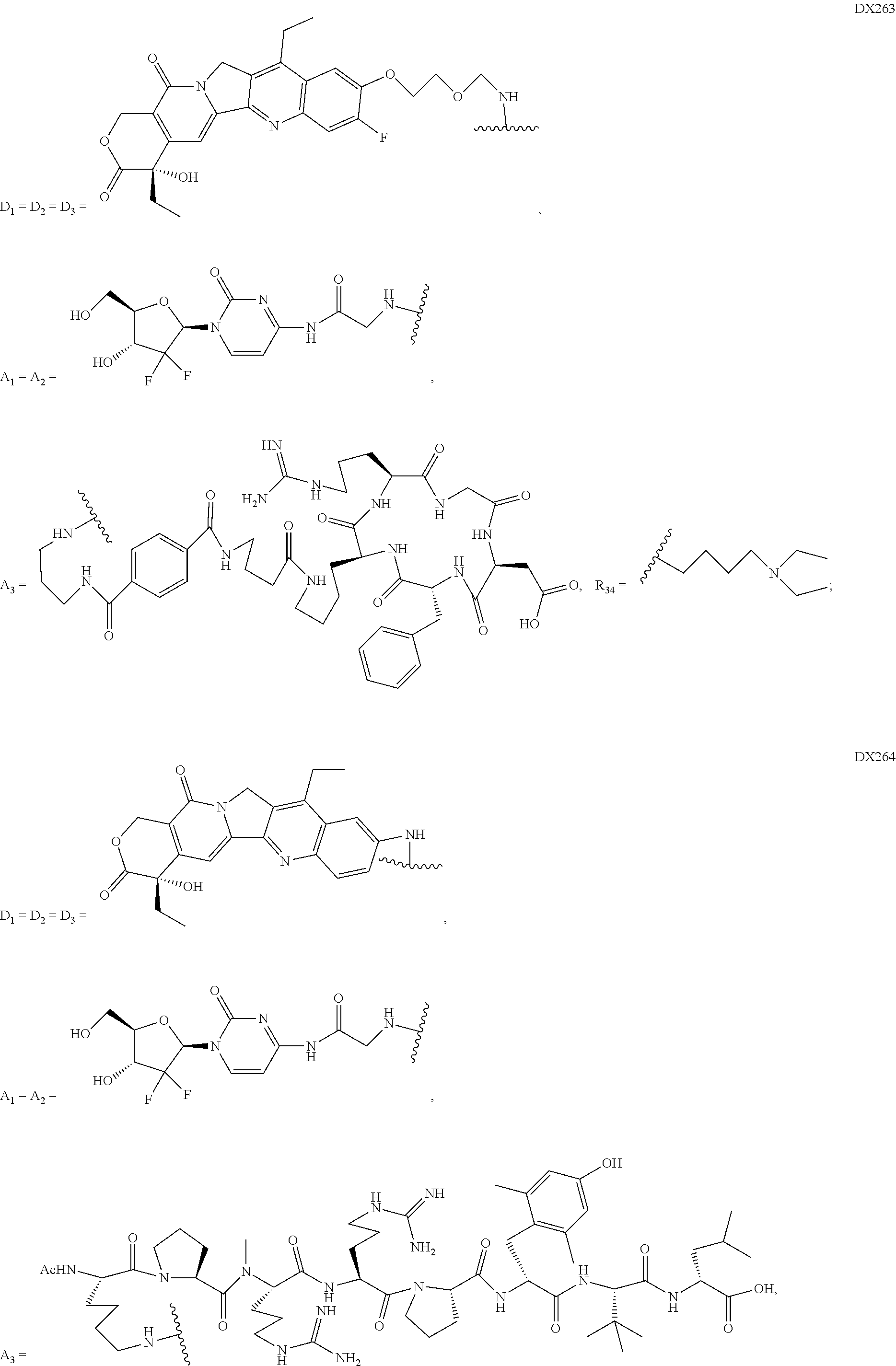

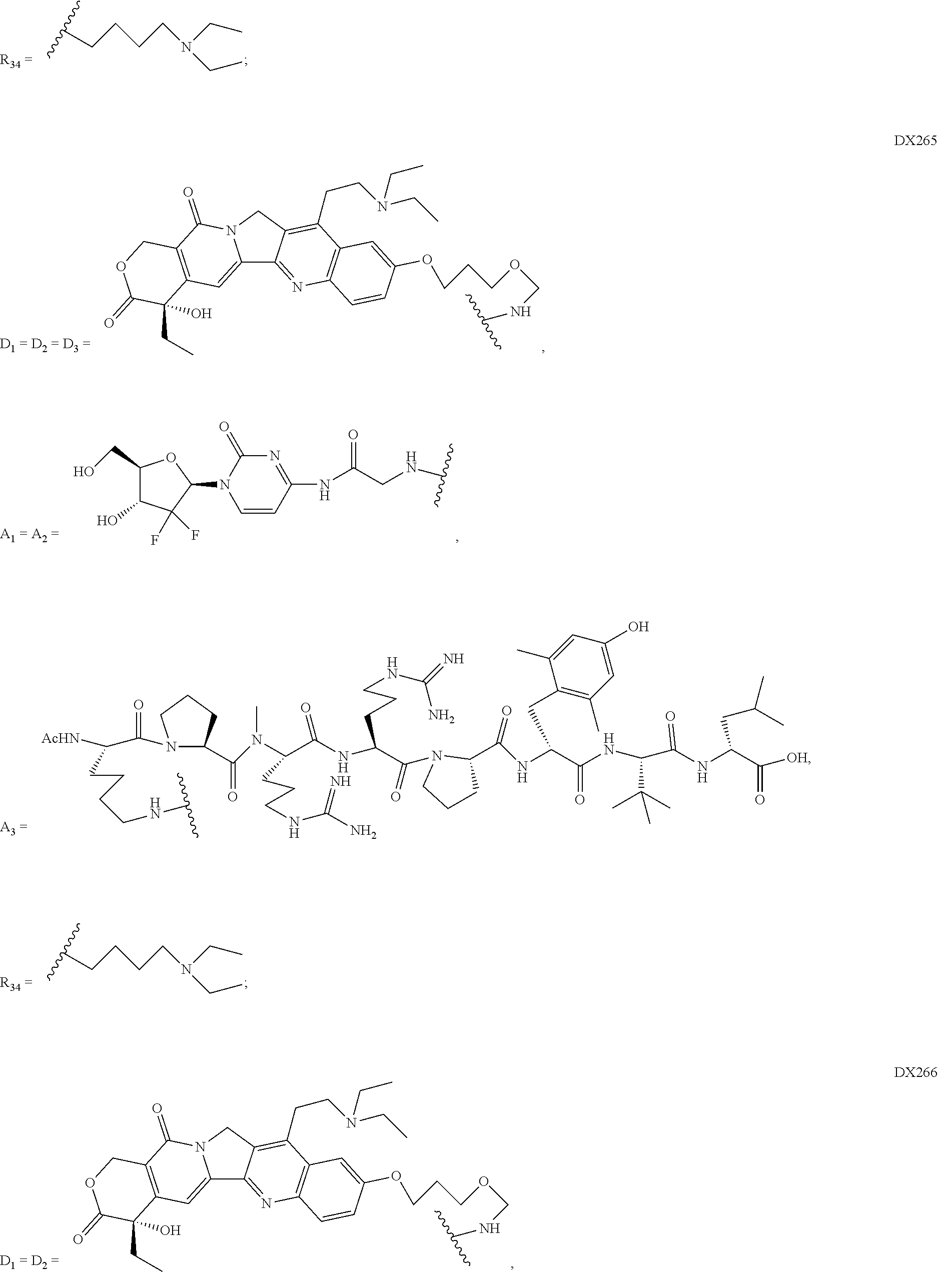

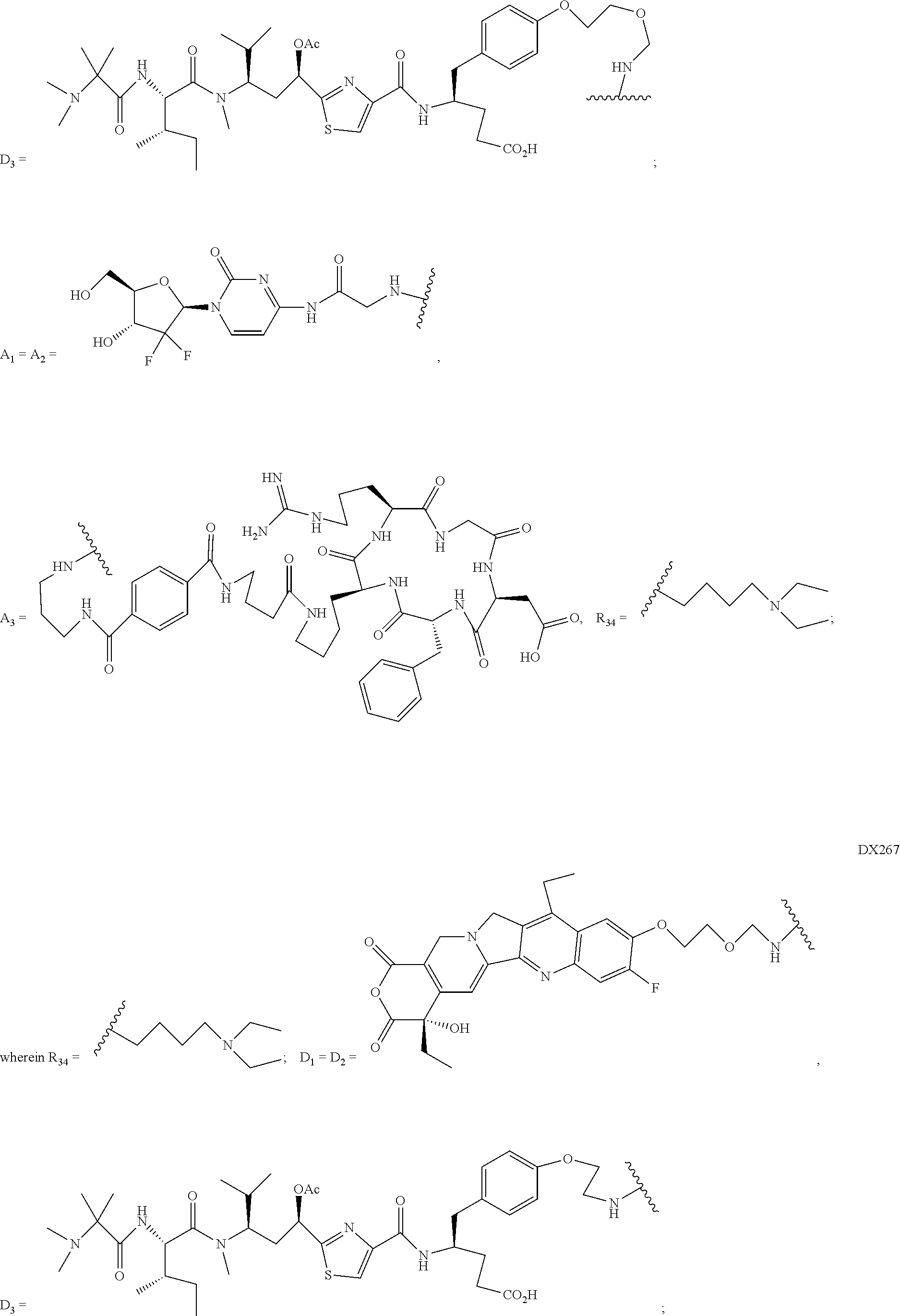

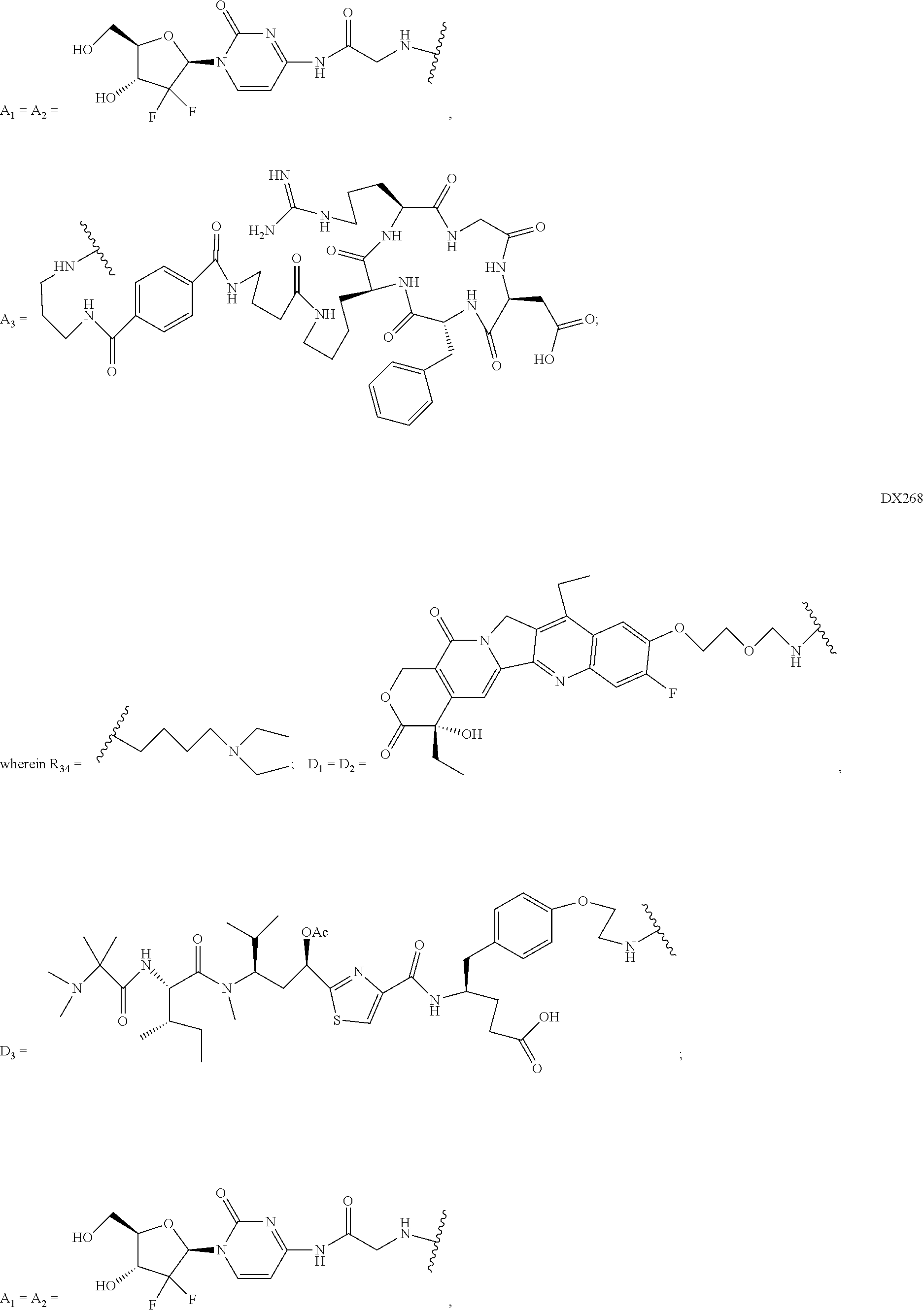

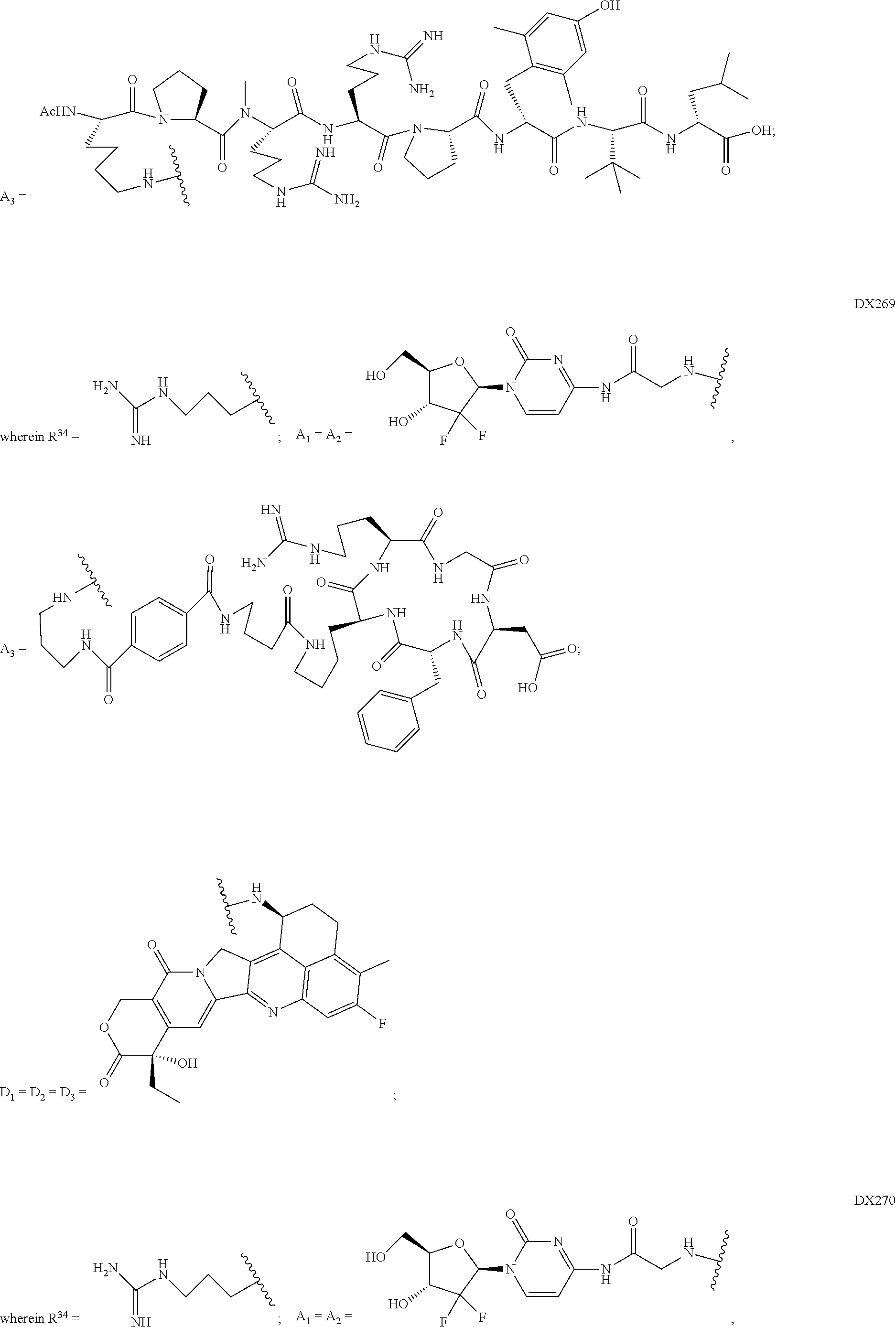

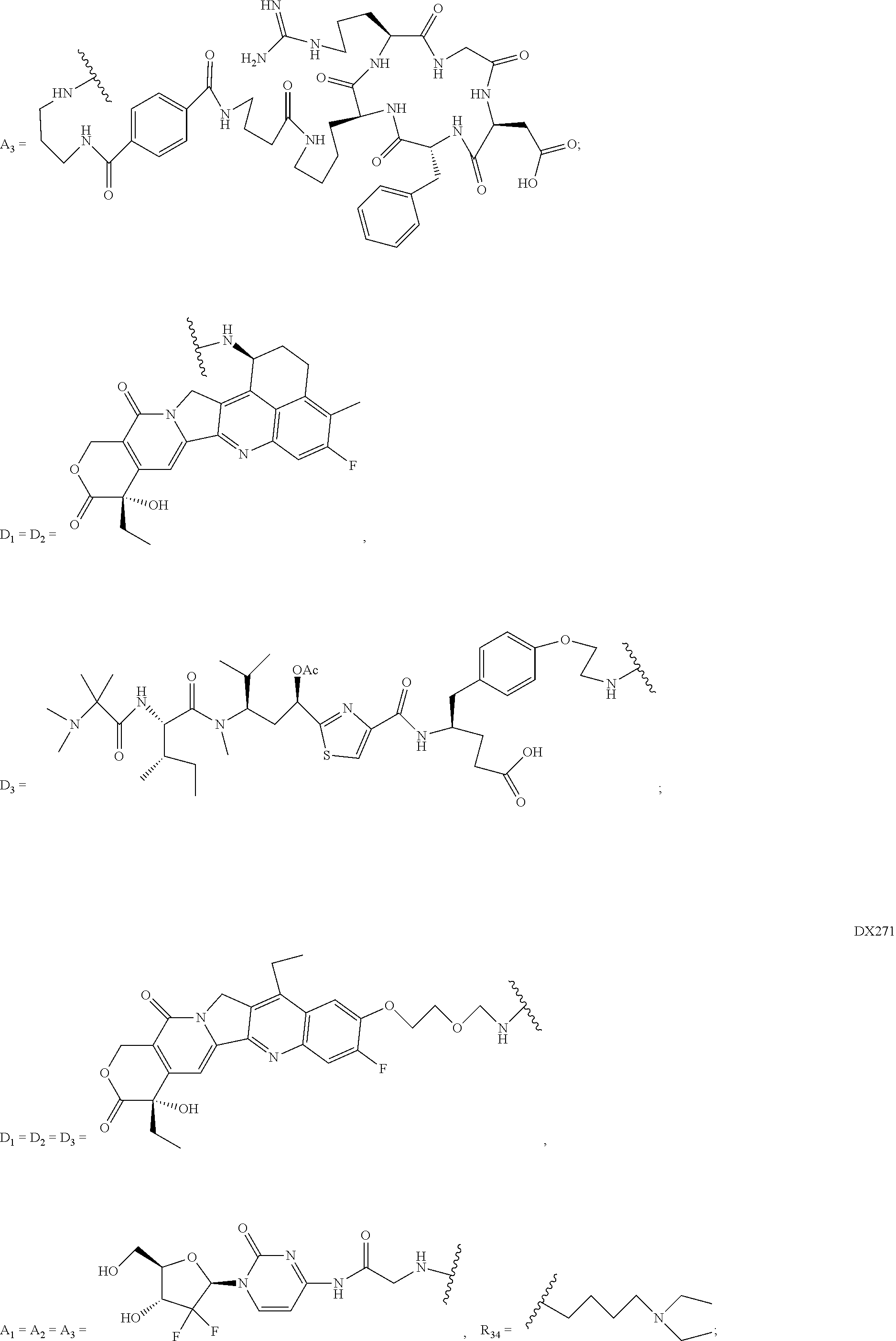

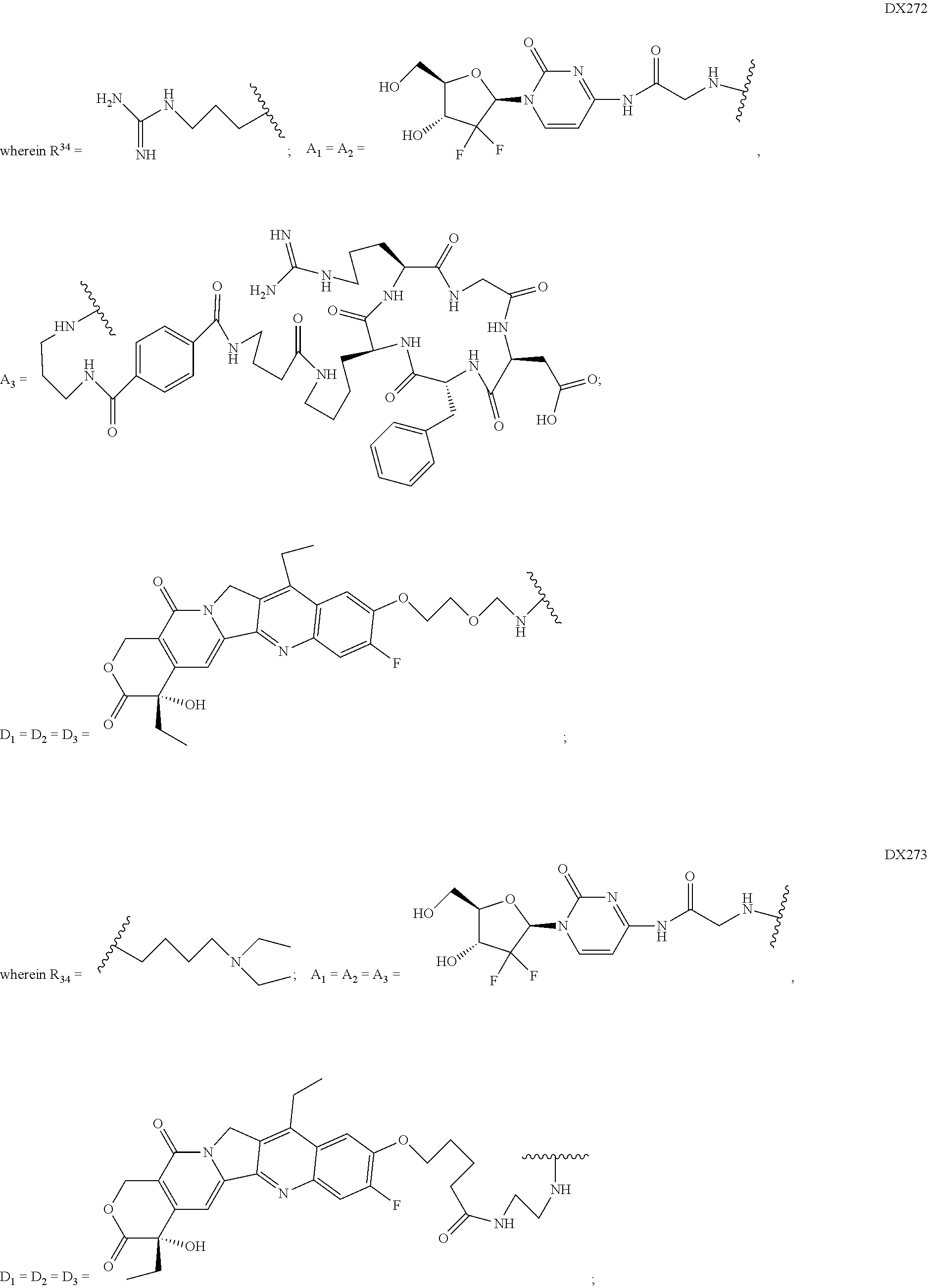

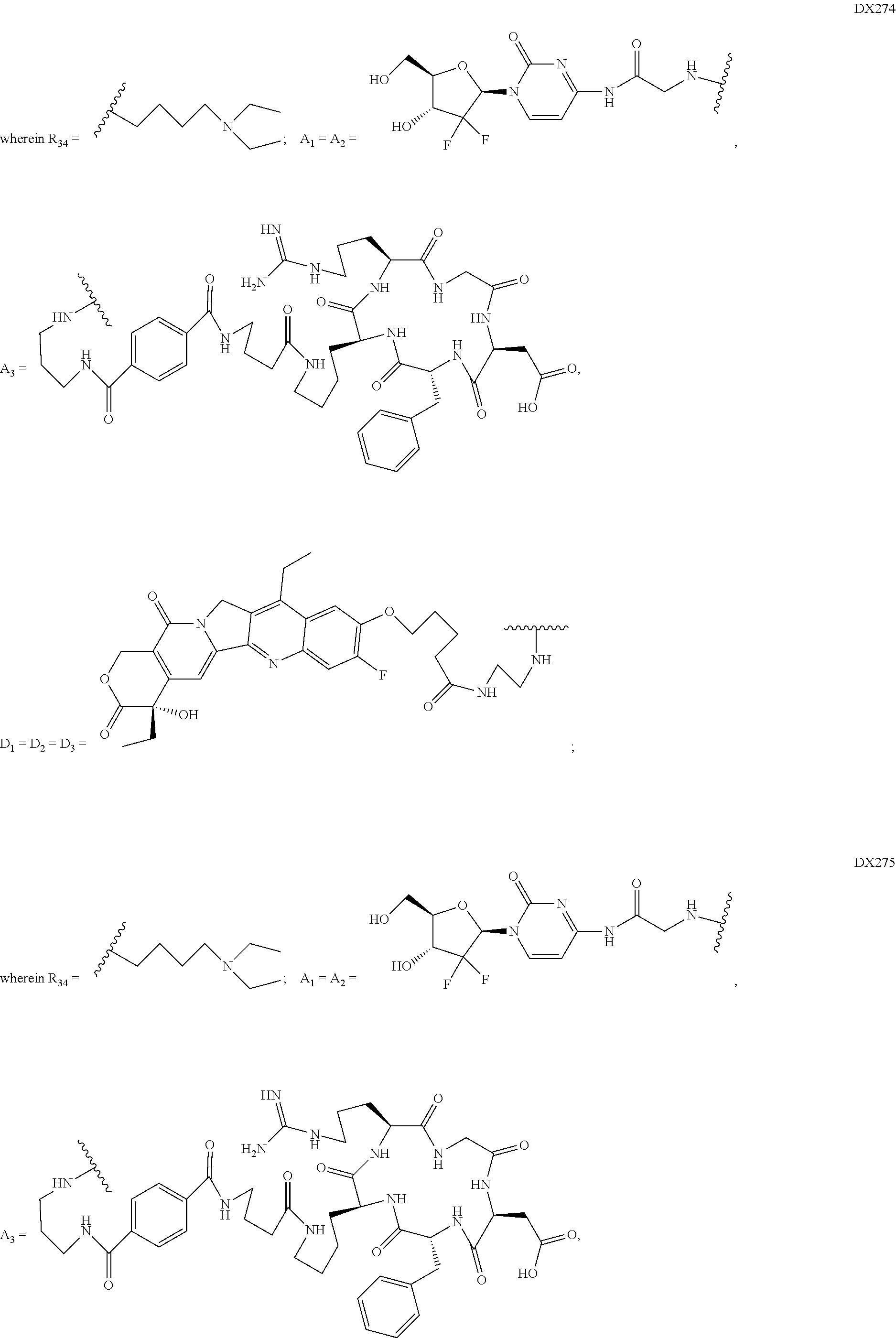

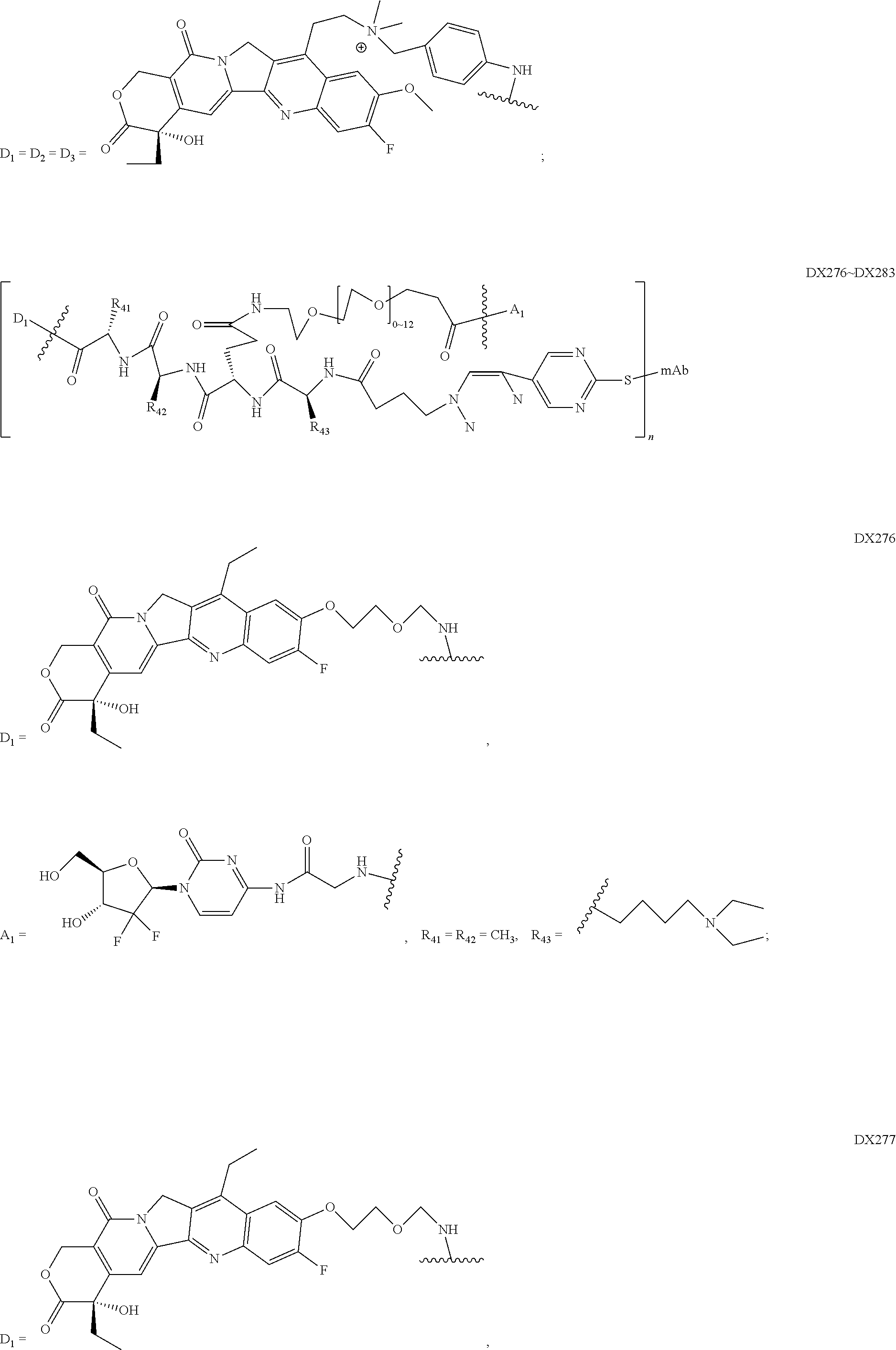

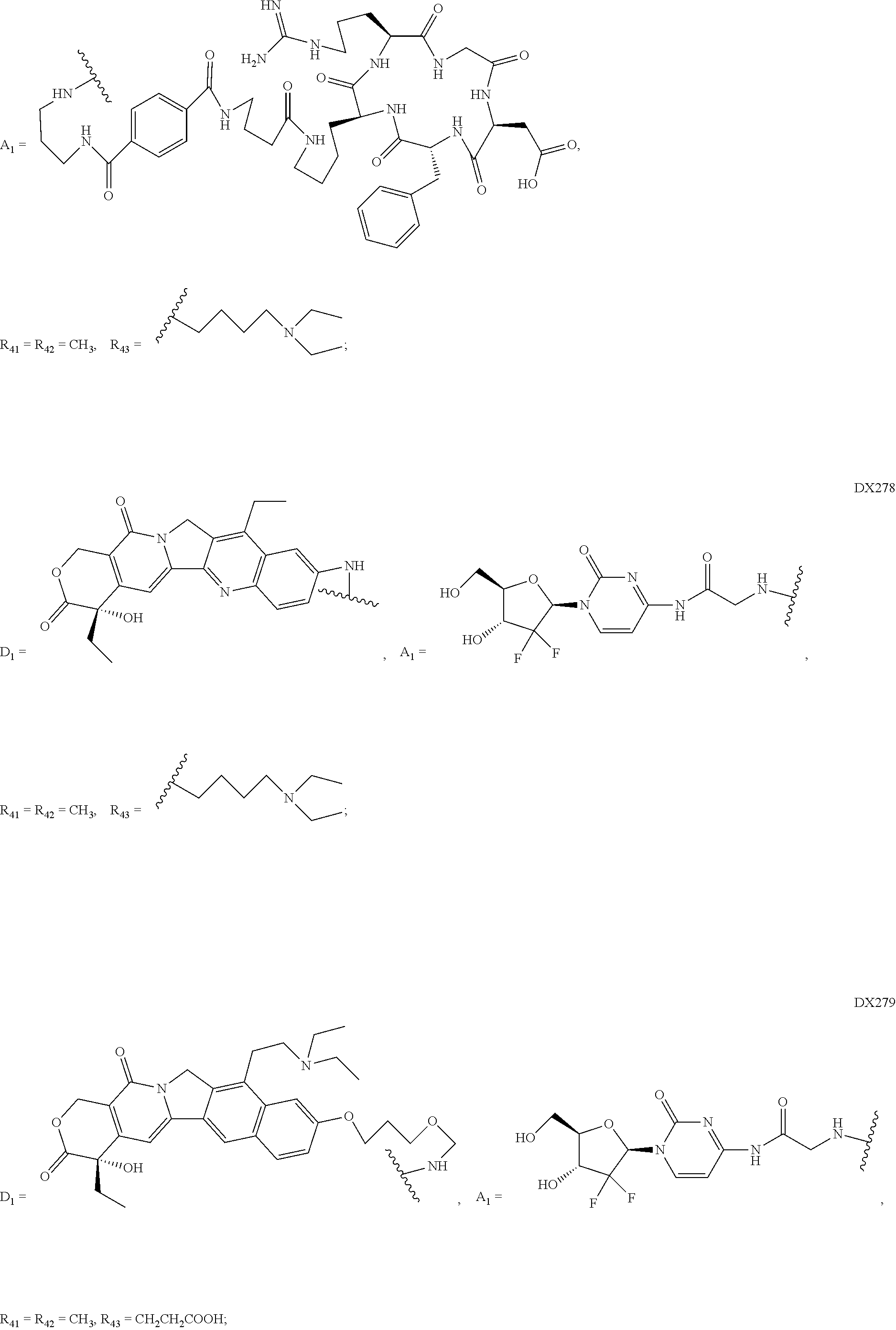

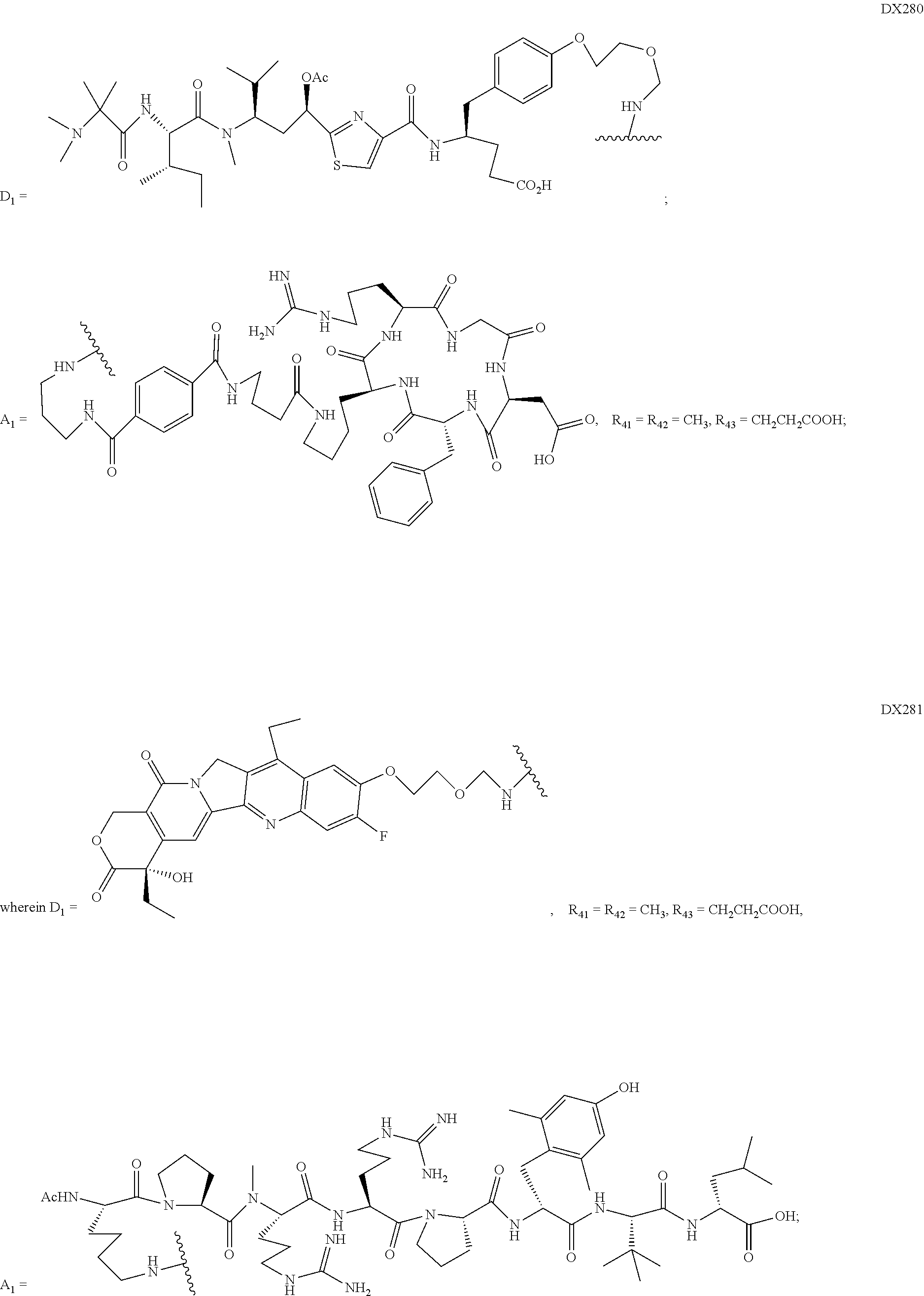

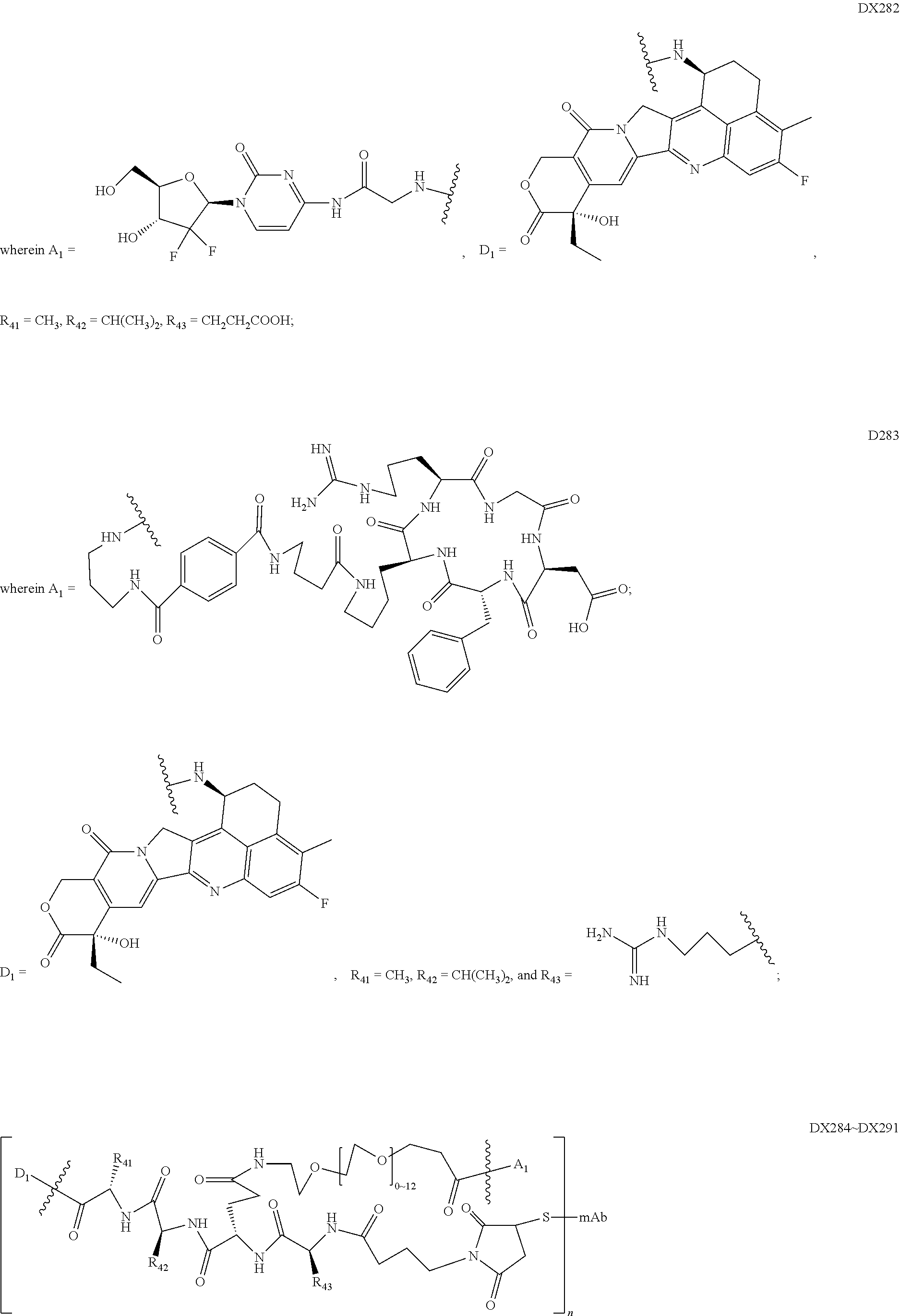

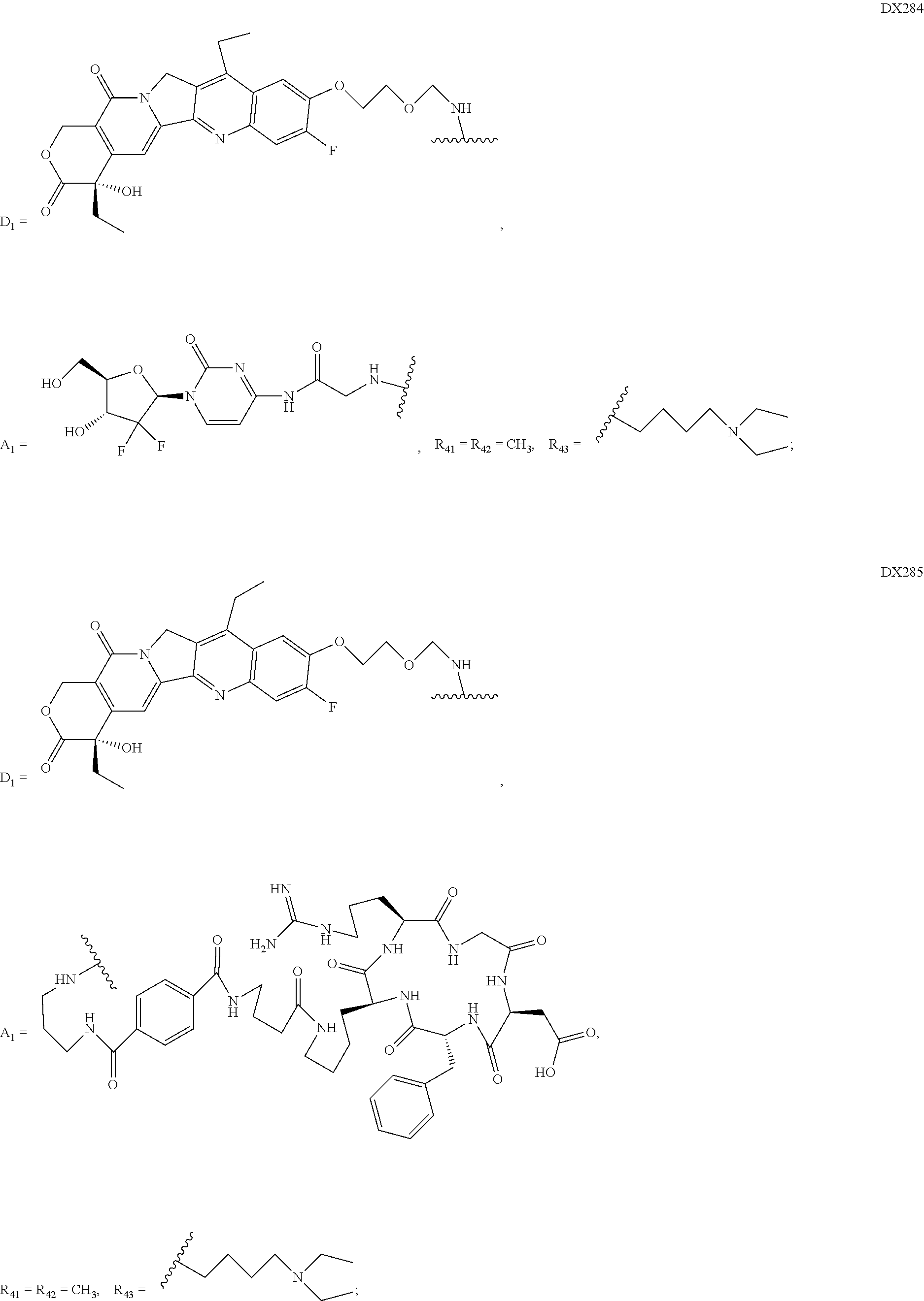

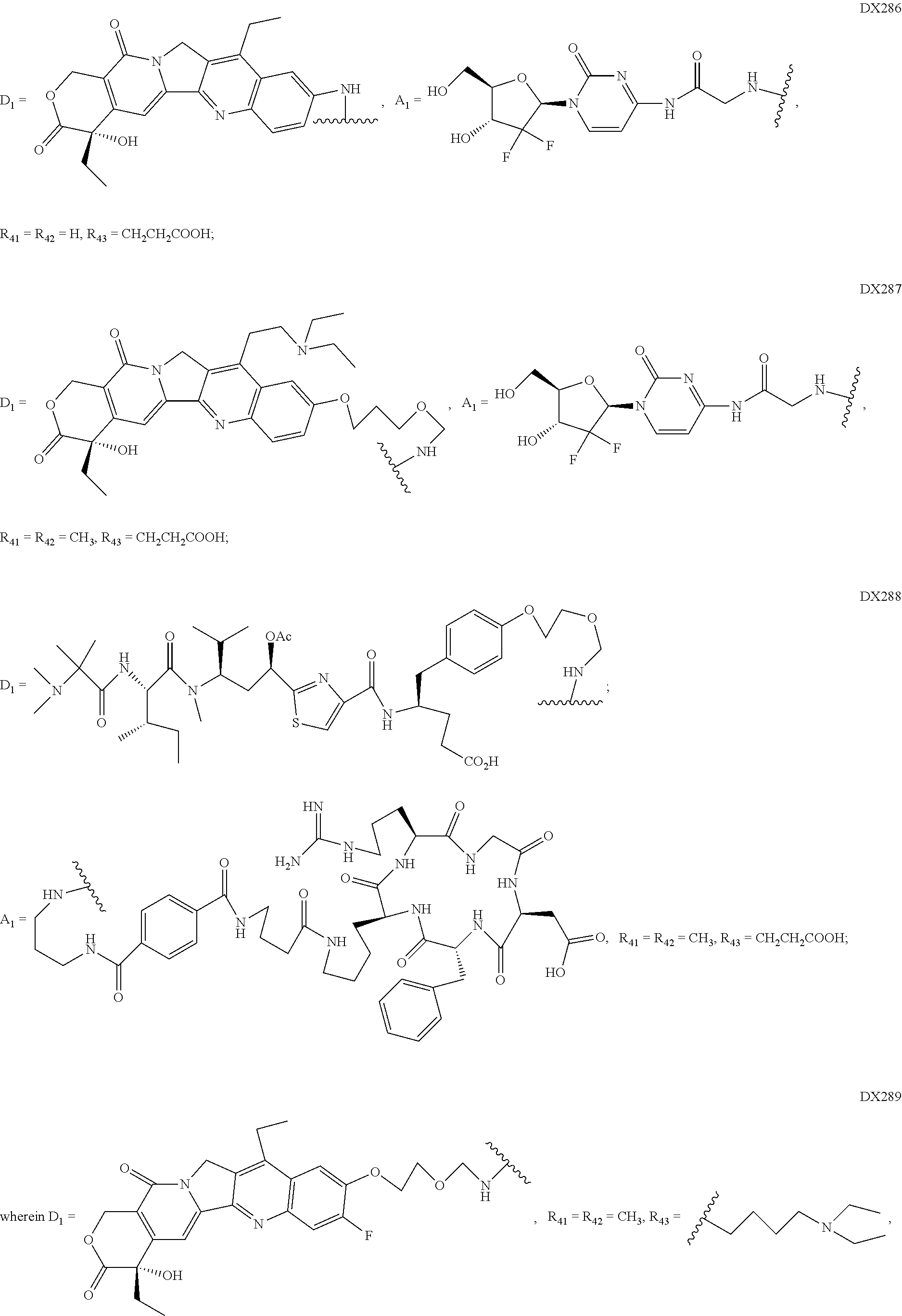

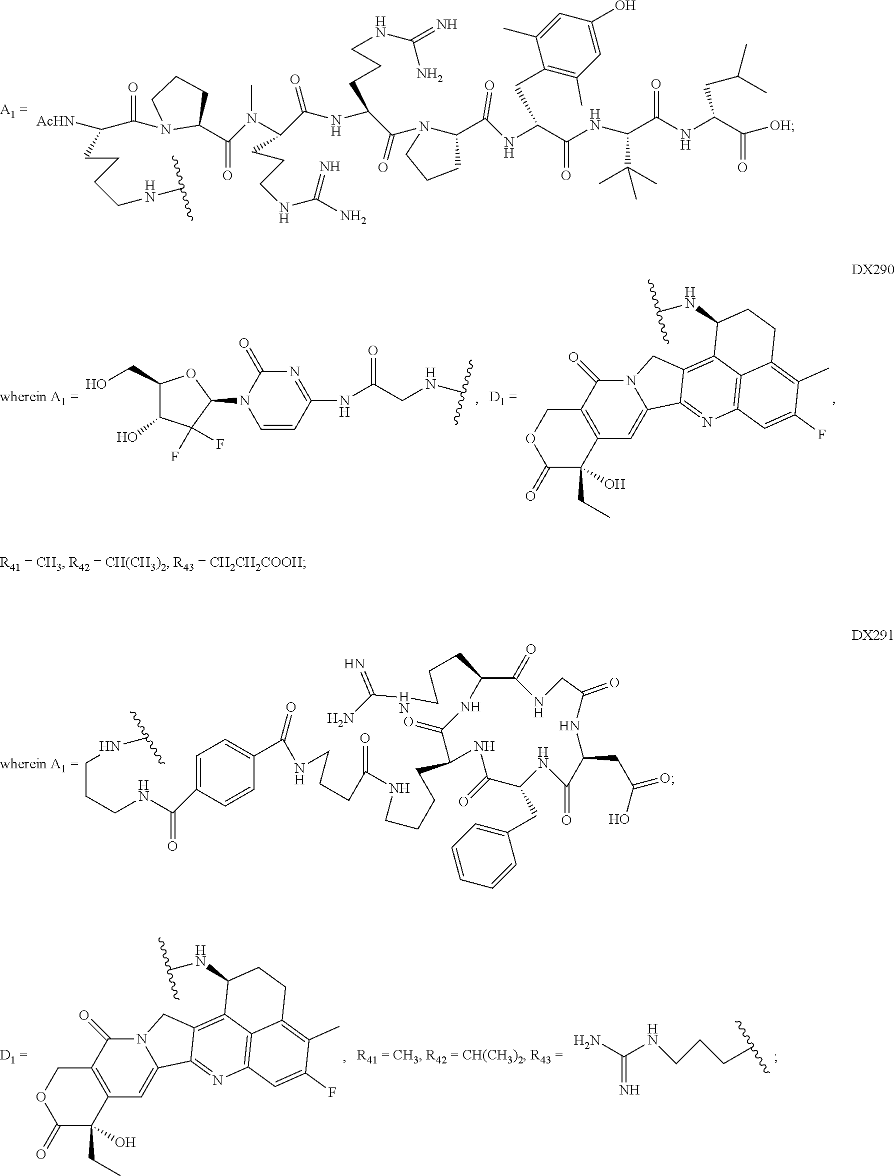

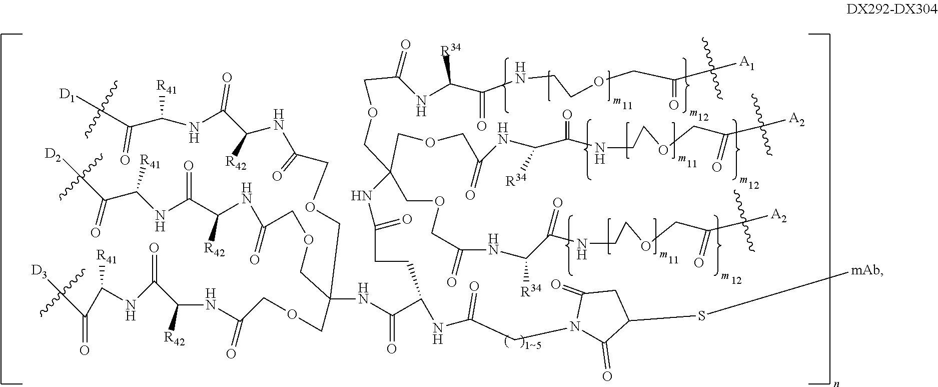

wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(—NH)NH₂, CH₂OH, CH(OH)CH₃, CH₂CH₂SCH₃, CH₂CH₂S(02)CH₃, CH₂SH, CH₂SO₃H, CH₂C₆H₅, and CH₂C₆H40H; R³⁴ is H, CH₃, CH₂CH₃, C(O)H, and C(O)CH₃; D1, D₂, D3 and D4 are cytotoxic drug described in this application, and A₁, A₂ and A₃ are small molecular ligands or drugs described in this application; mn is a number of 0, 1, 2, 3, 4, 5, 6, 7, 8, 9 and m₁₂ is a number of 0, 1, 2, 3 and 4; when number is 0 for the brackets, it means the structure in the brackets can be omitted (crossed out),

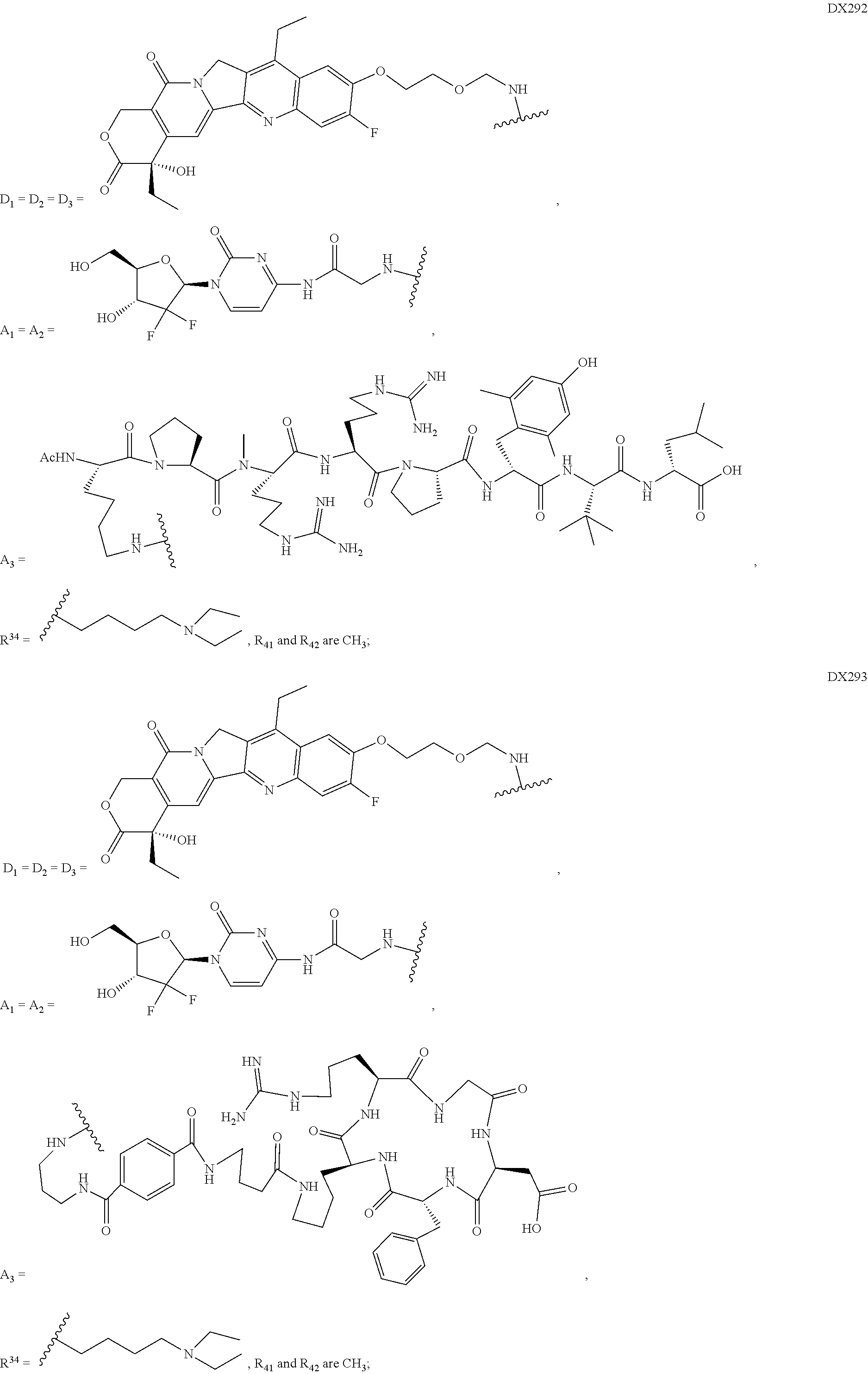

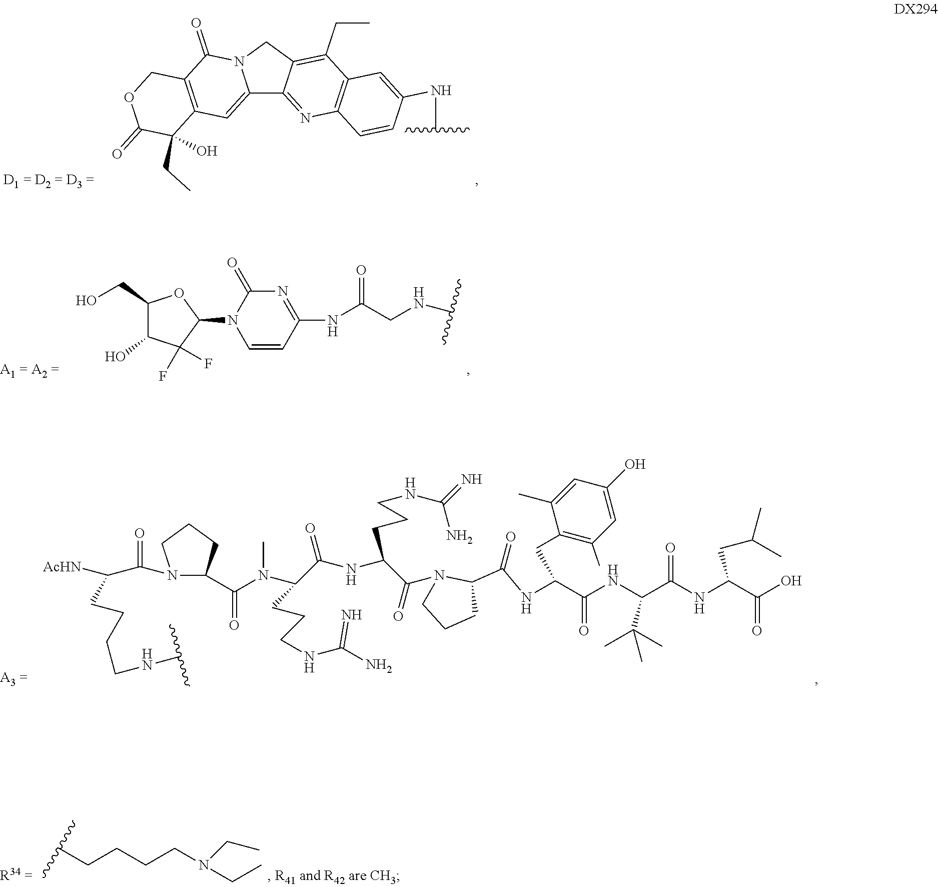

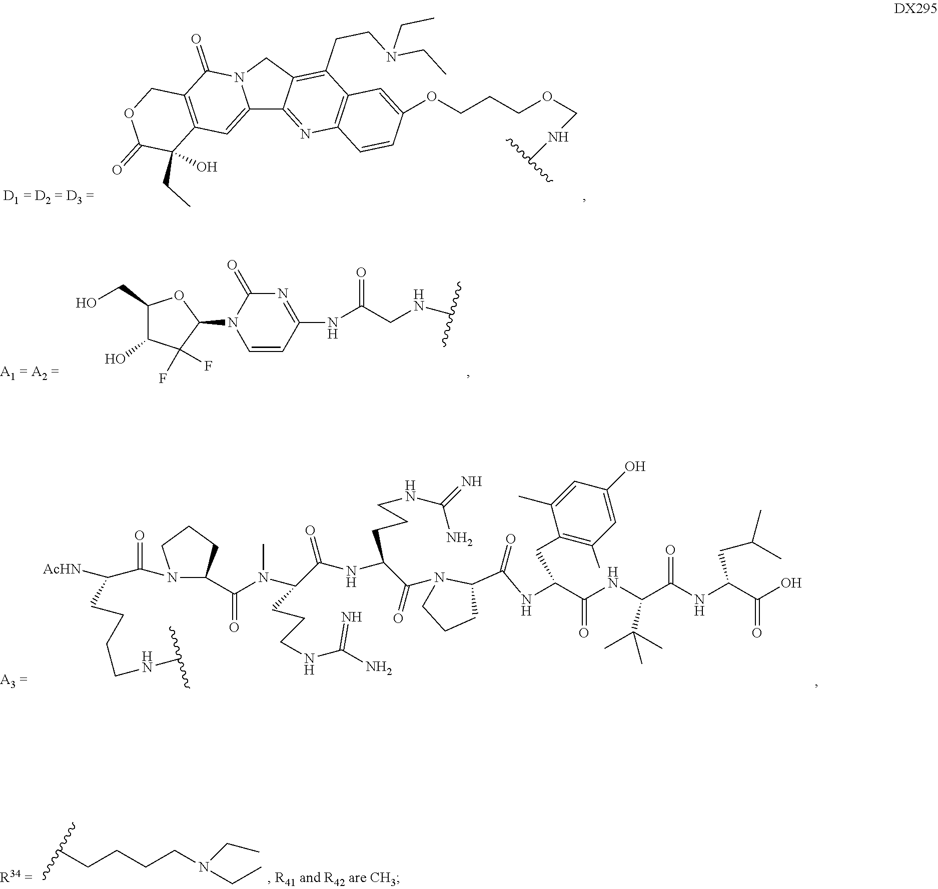

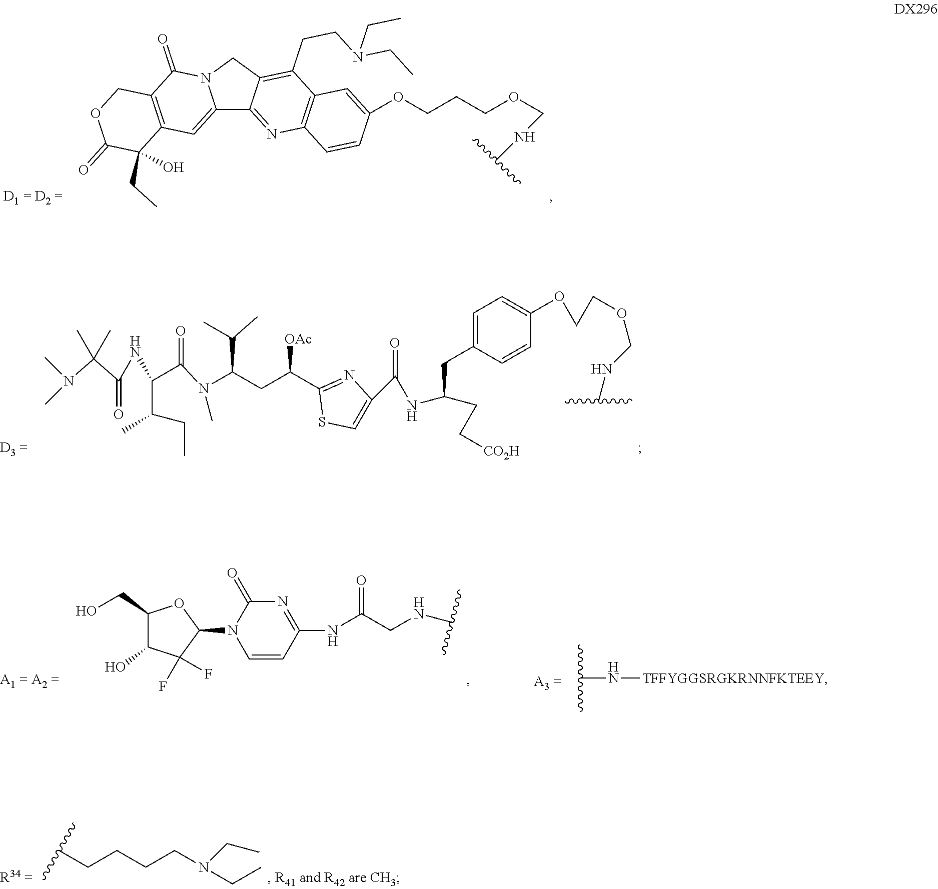

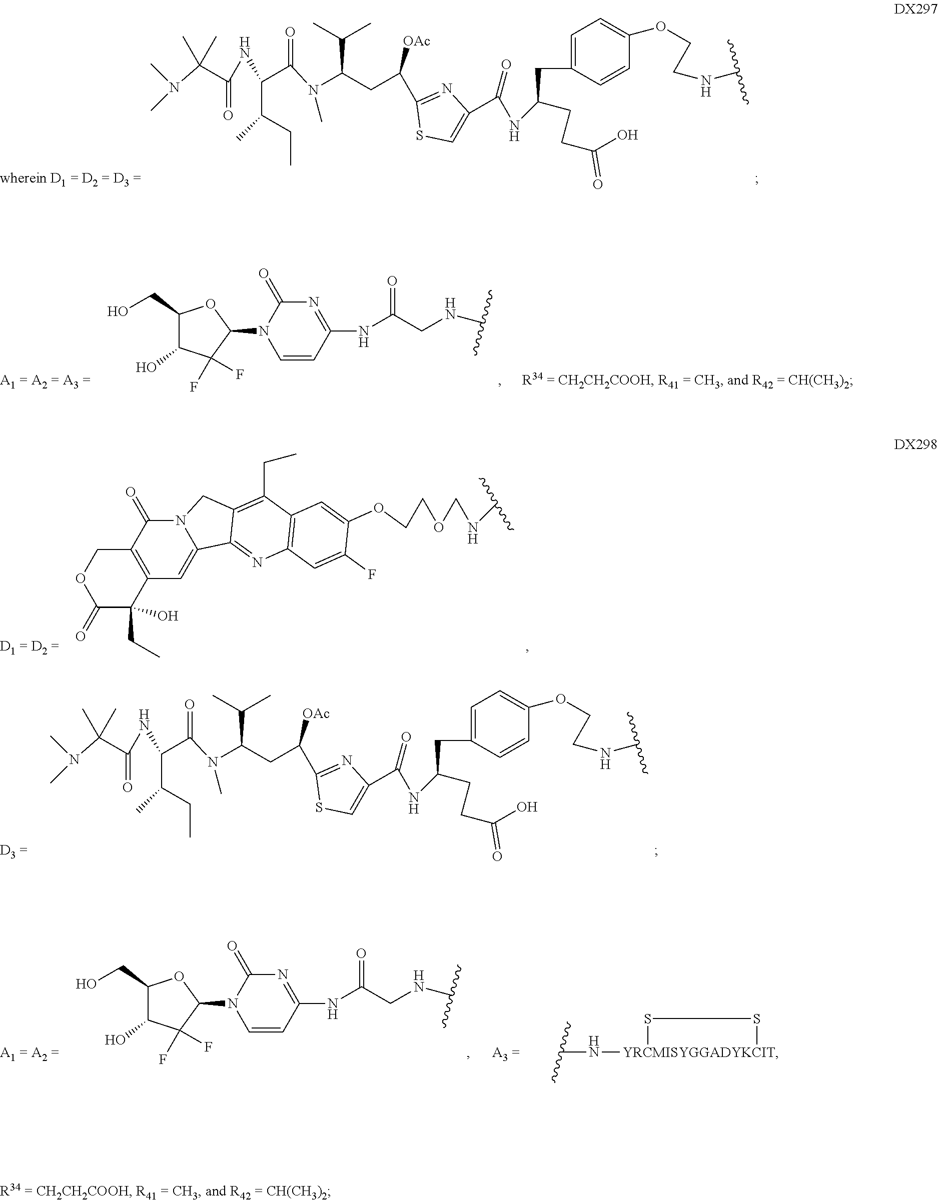

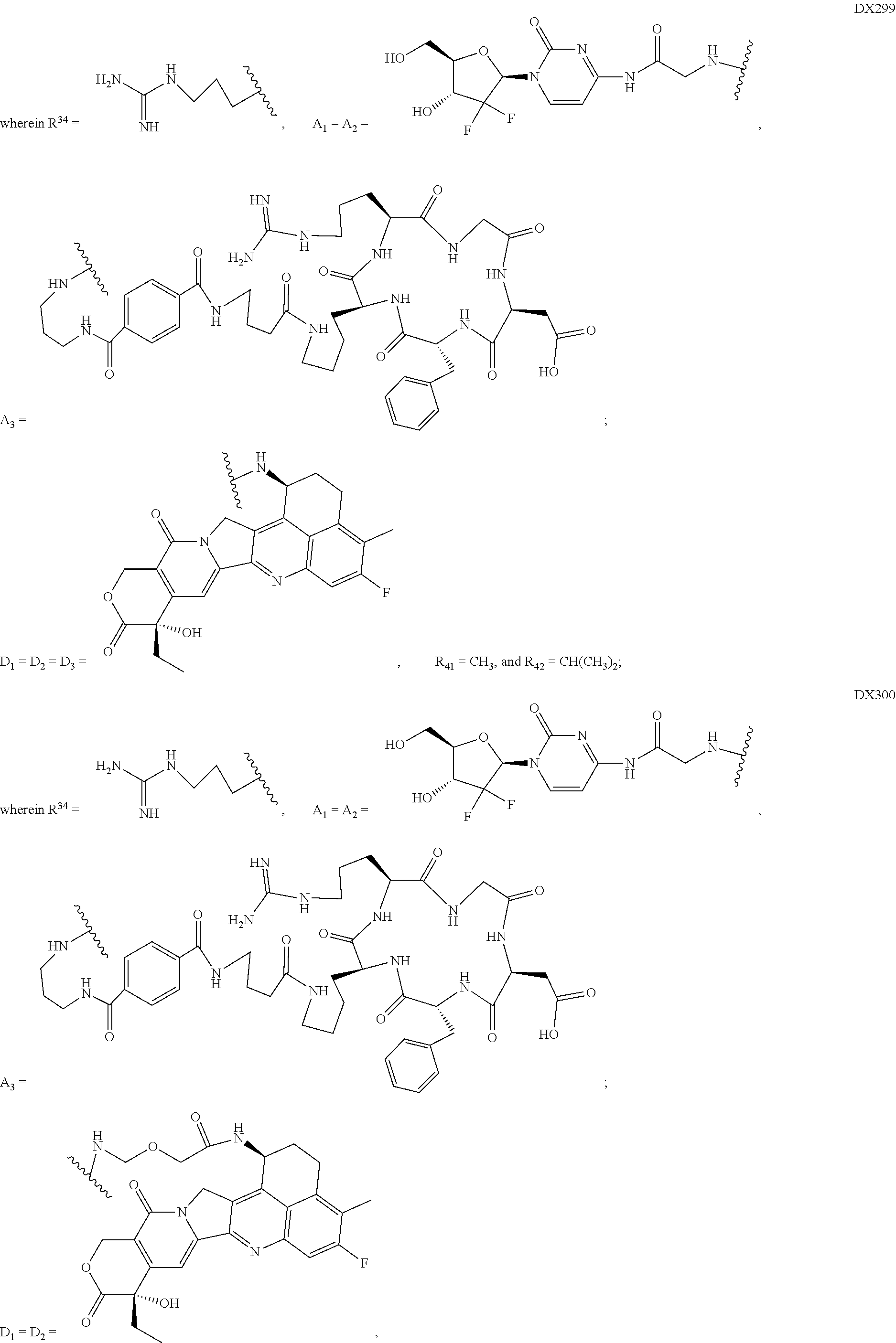

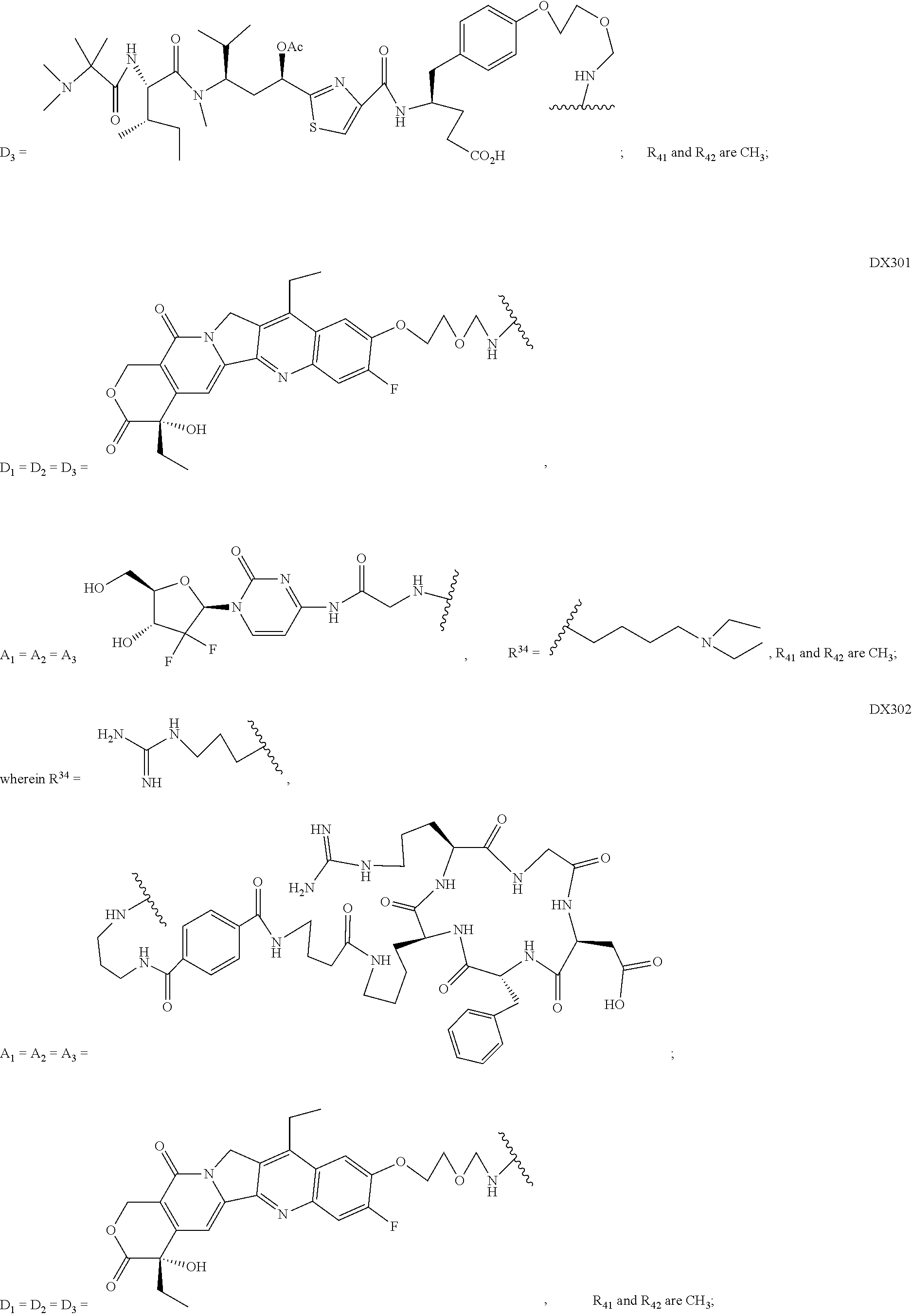

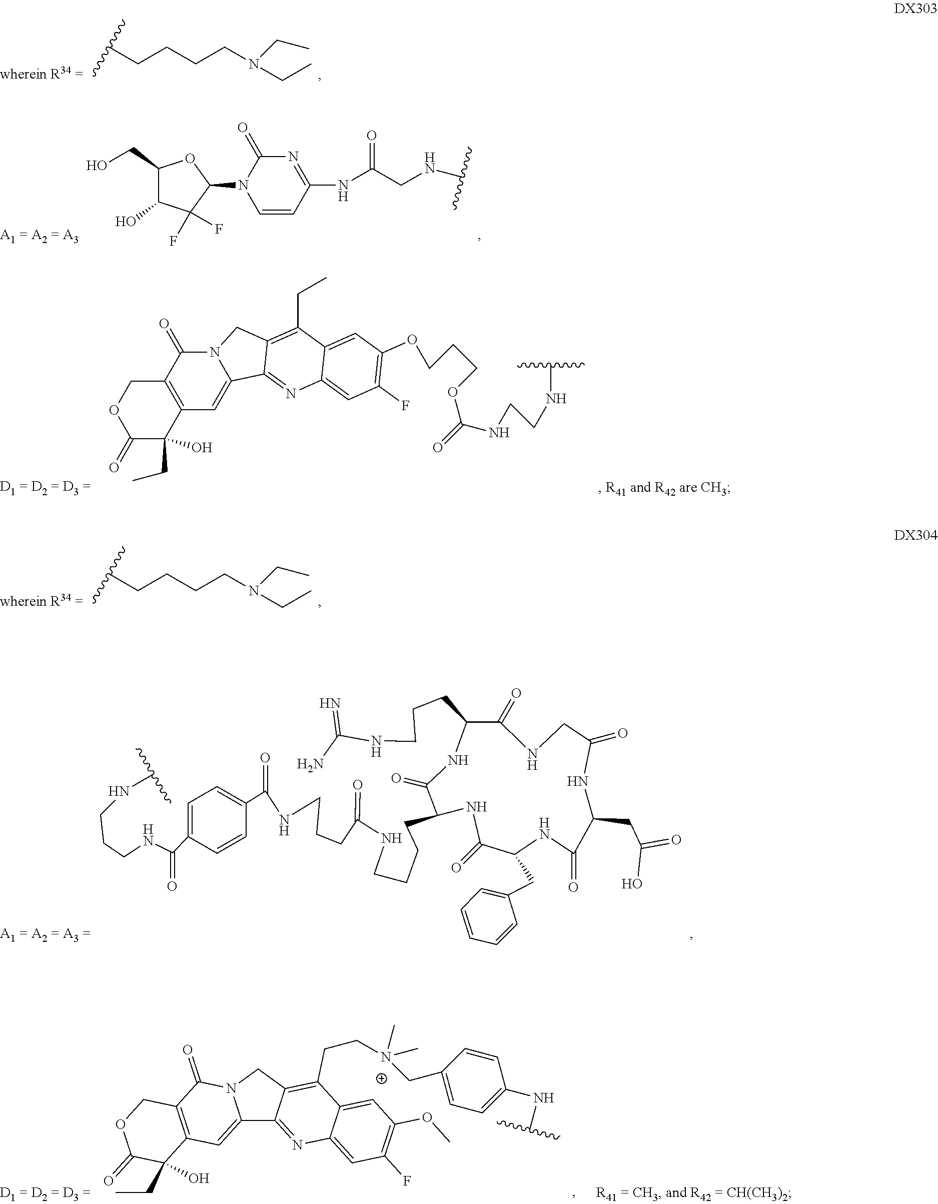

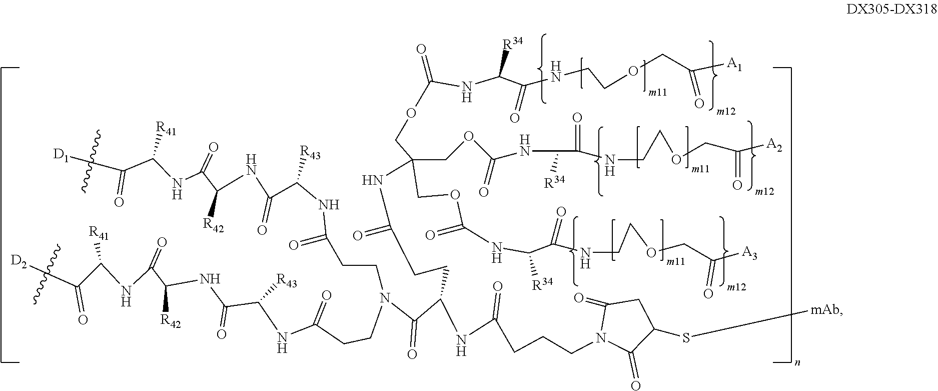

wherein R₄ and R₄₂ are independently H, CH₃, CH(CH₃)₂, CH(CH₃)(C₂H), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(02)CH₃, CH₂SH, CH₂SO₃H, CH₂C₆Hs, and CH₂C₆H40H; R³⁴ is H, CH₃, CH₂CH₃, C(O)H, and C(O)CH₃; D₁, D₂, D₃ and D₄ are cytotoxic drug described in this application, and A₁, A₂ and A₃ are small molecular ligands or drugs described in this application; mn is a number of 0, 1, 2, 3, 4, 5, 6, 7, 8, 9 and m₁₂ is a number of 0, 1, 2, 3 and 4; when number is 0, it means the structure in the brackets can be omitted (crossed out).

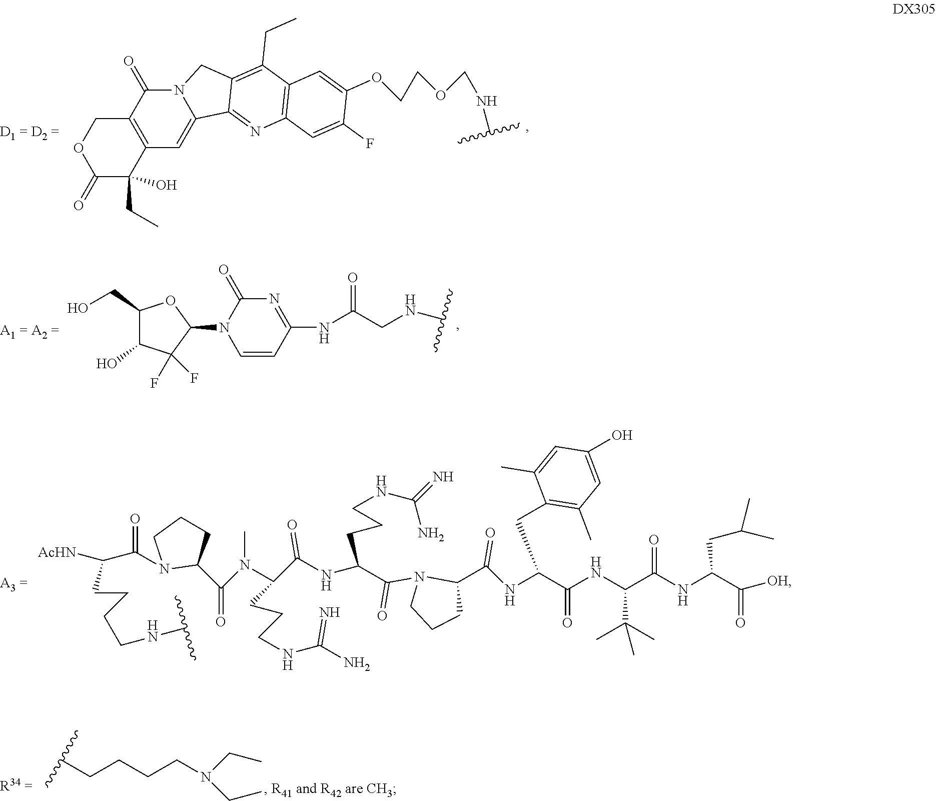

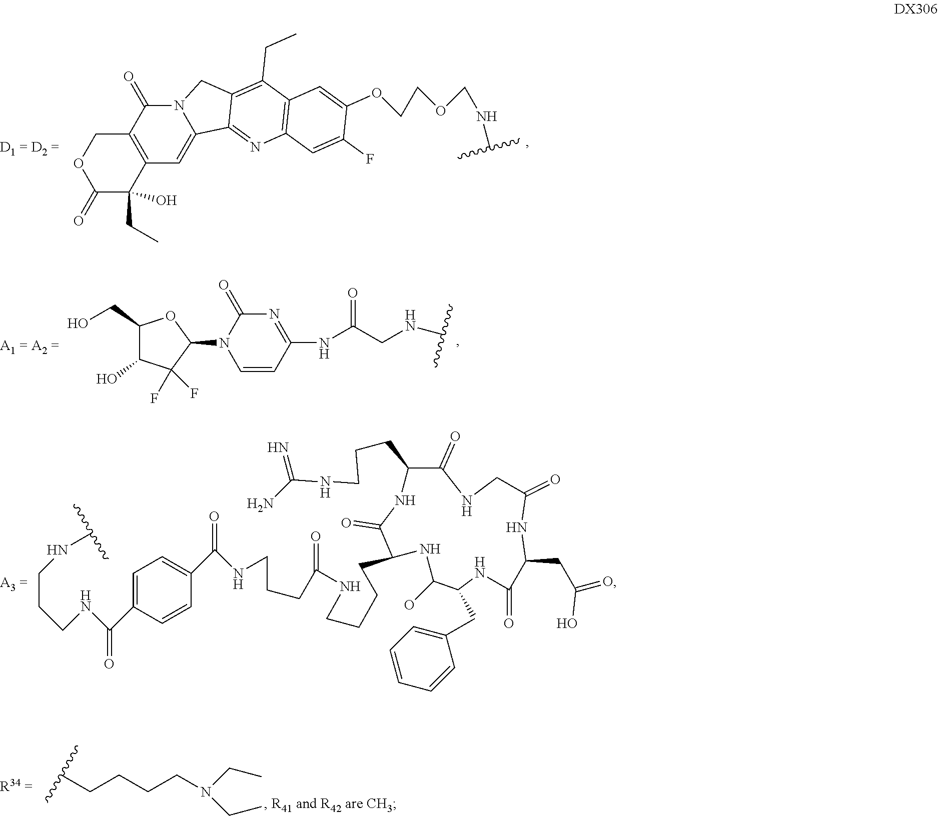

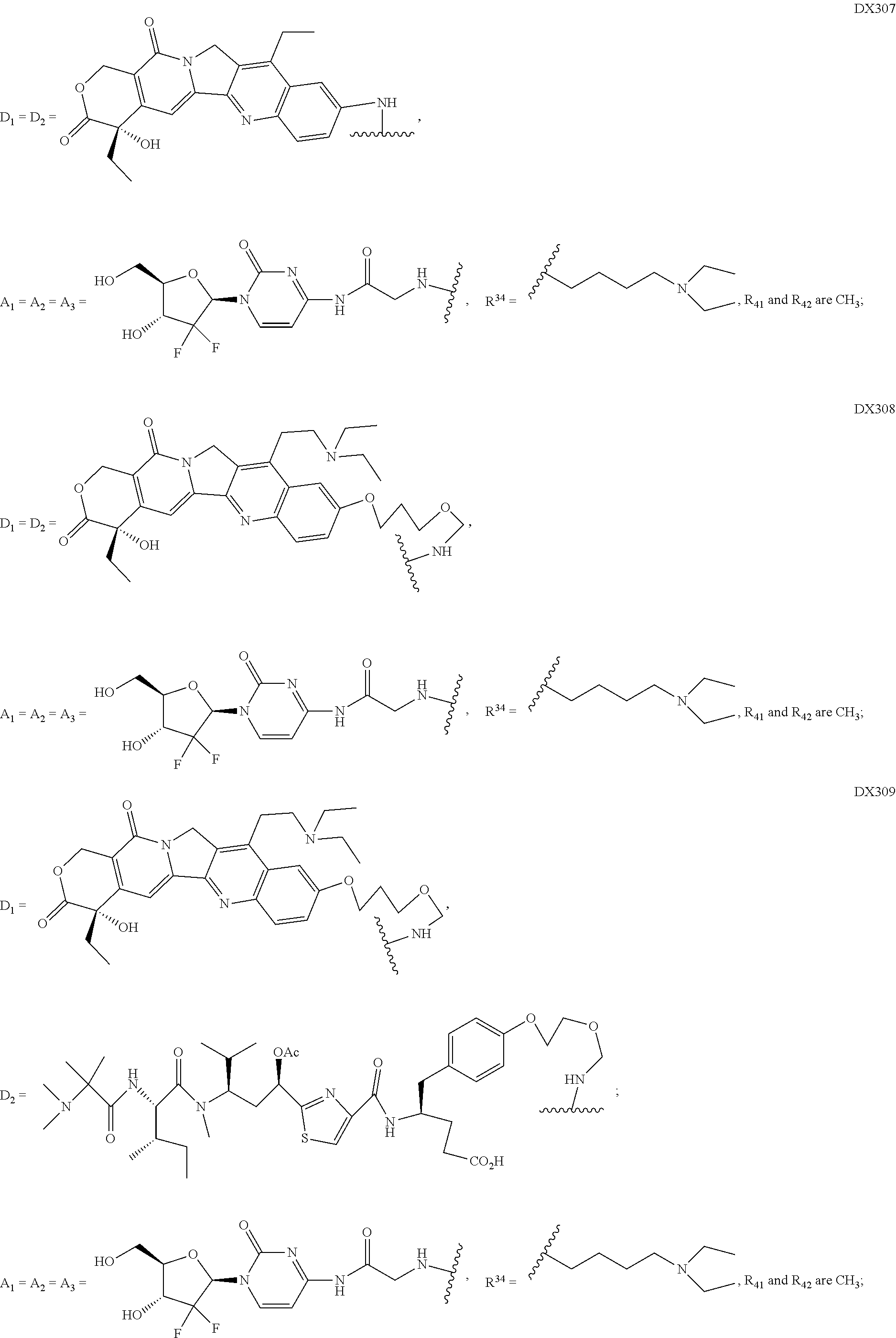

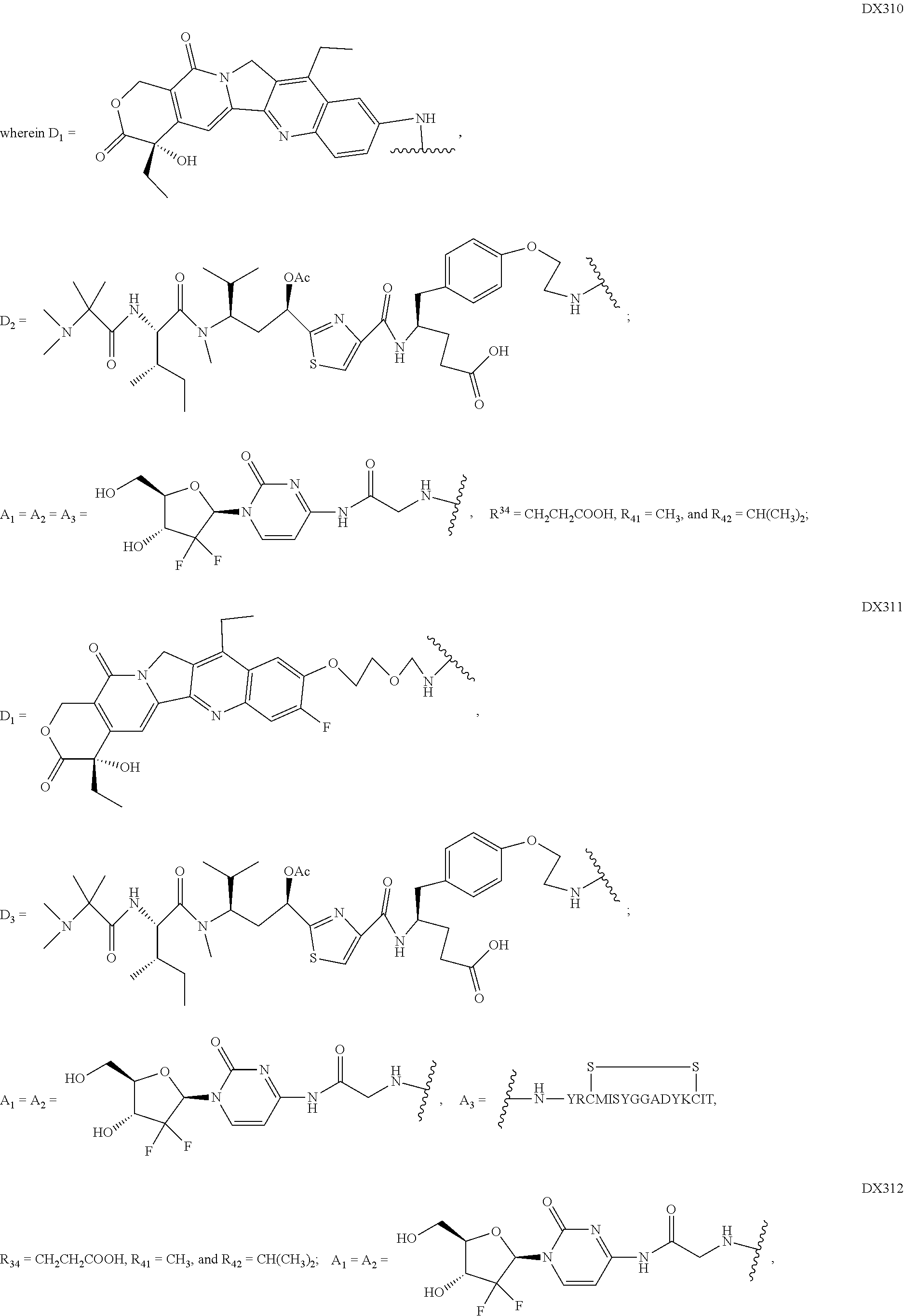

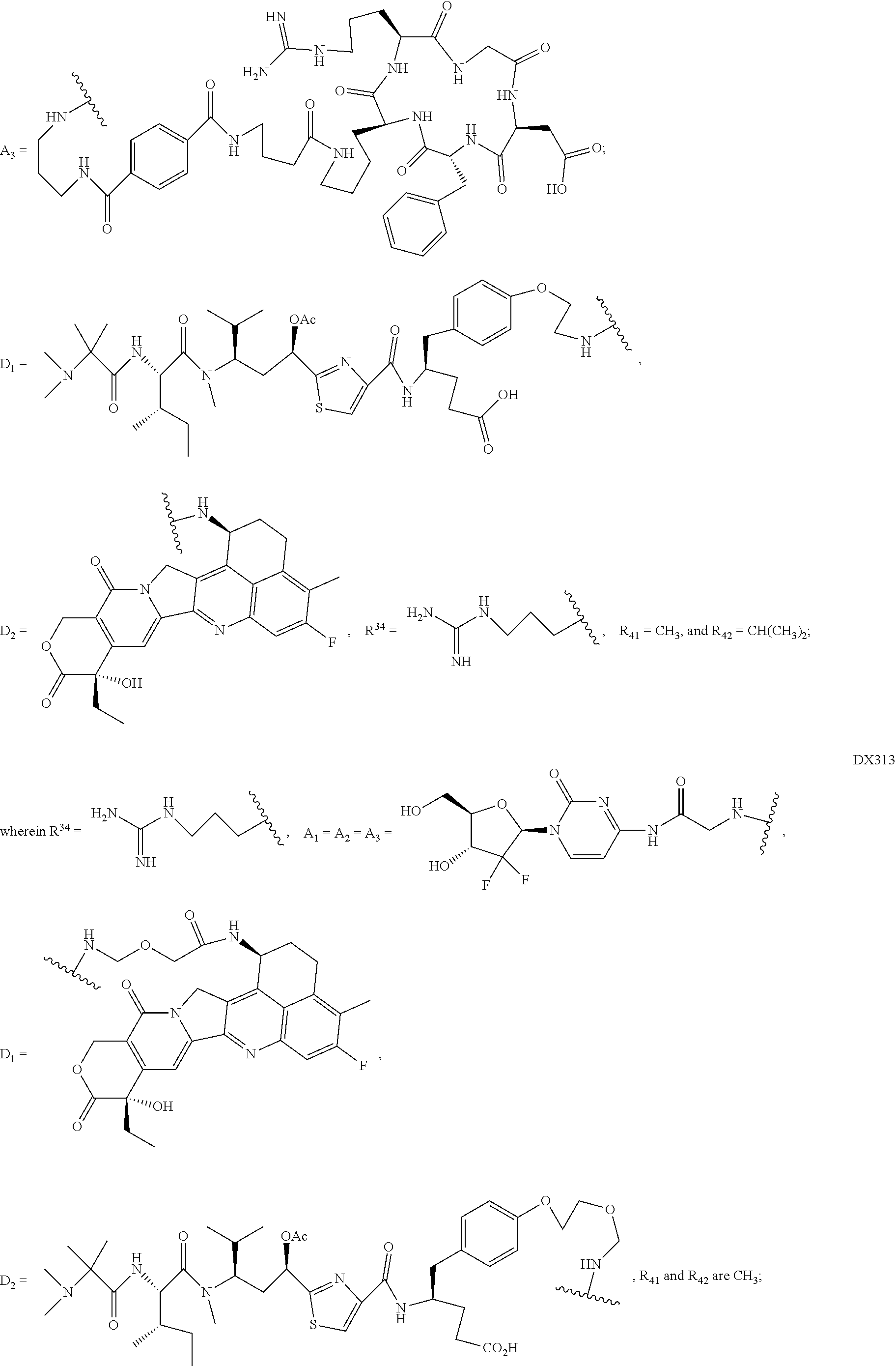

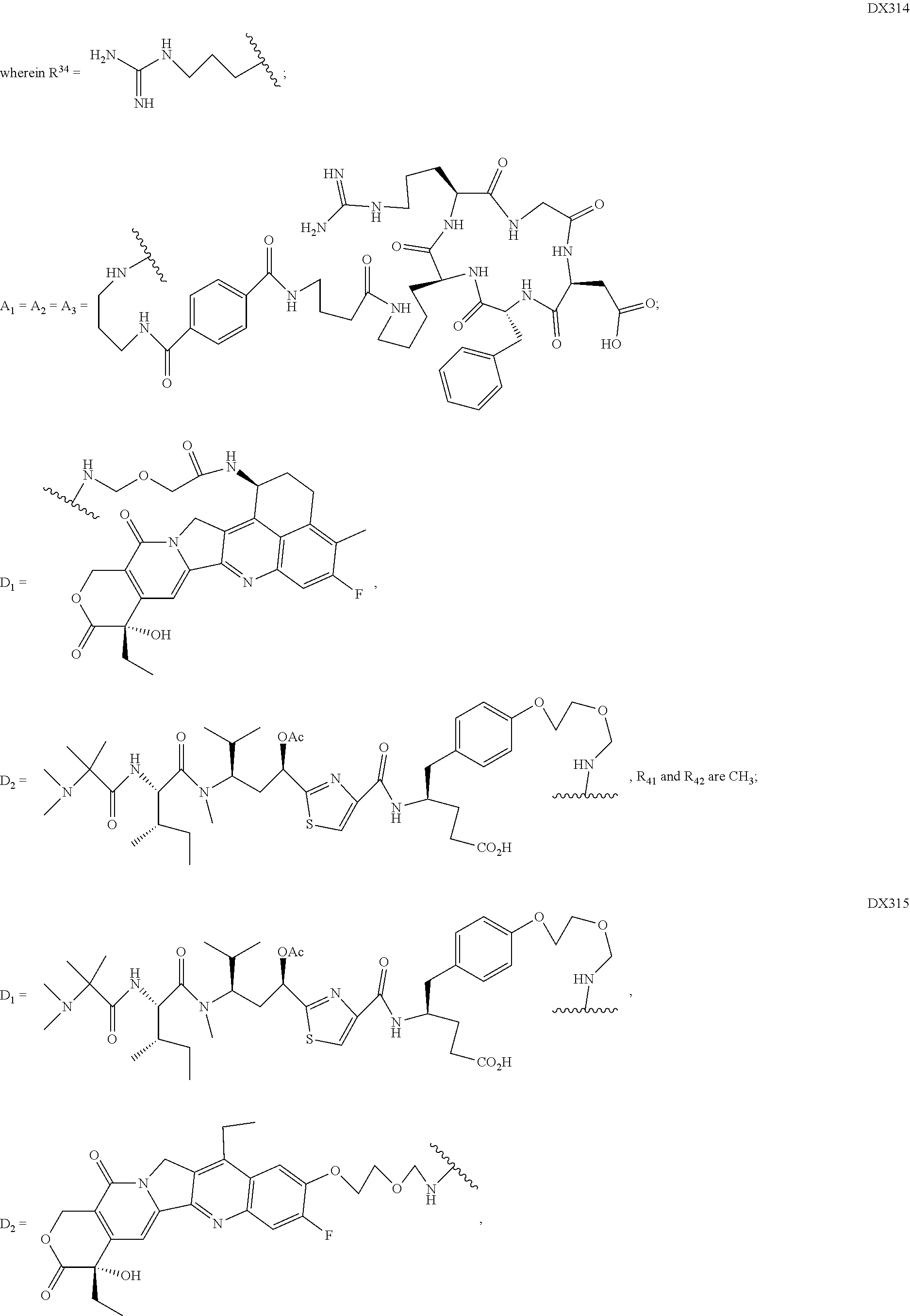

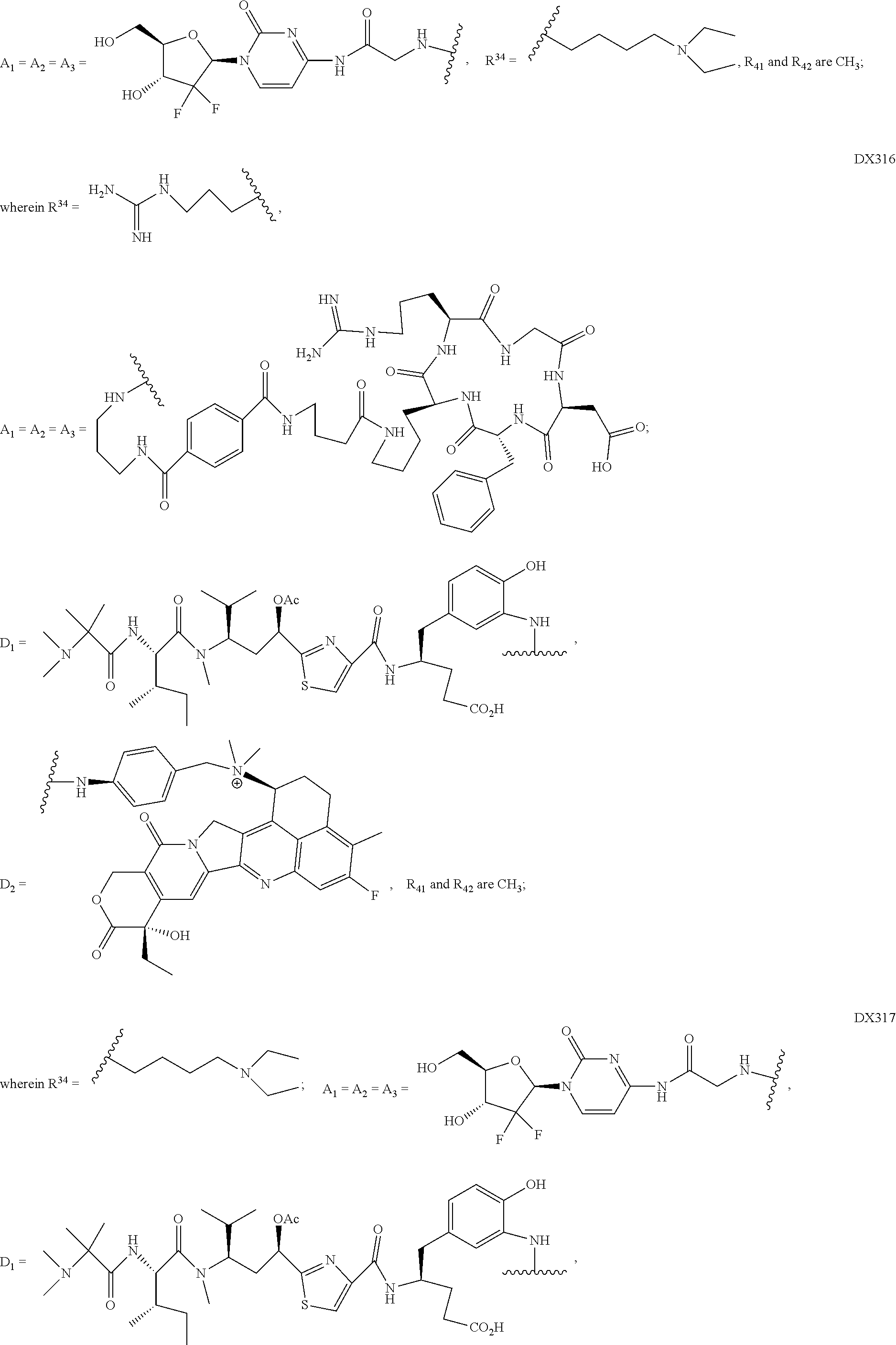

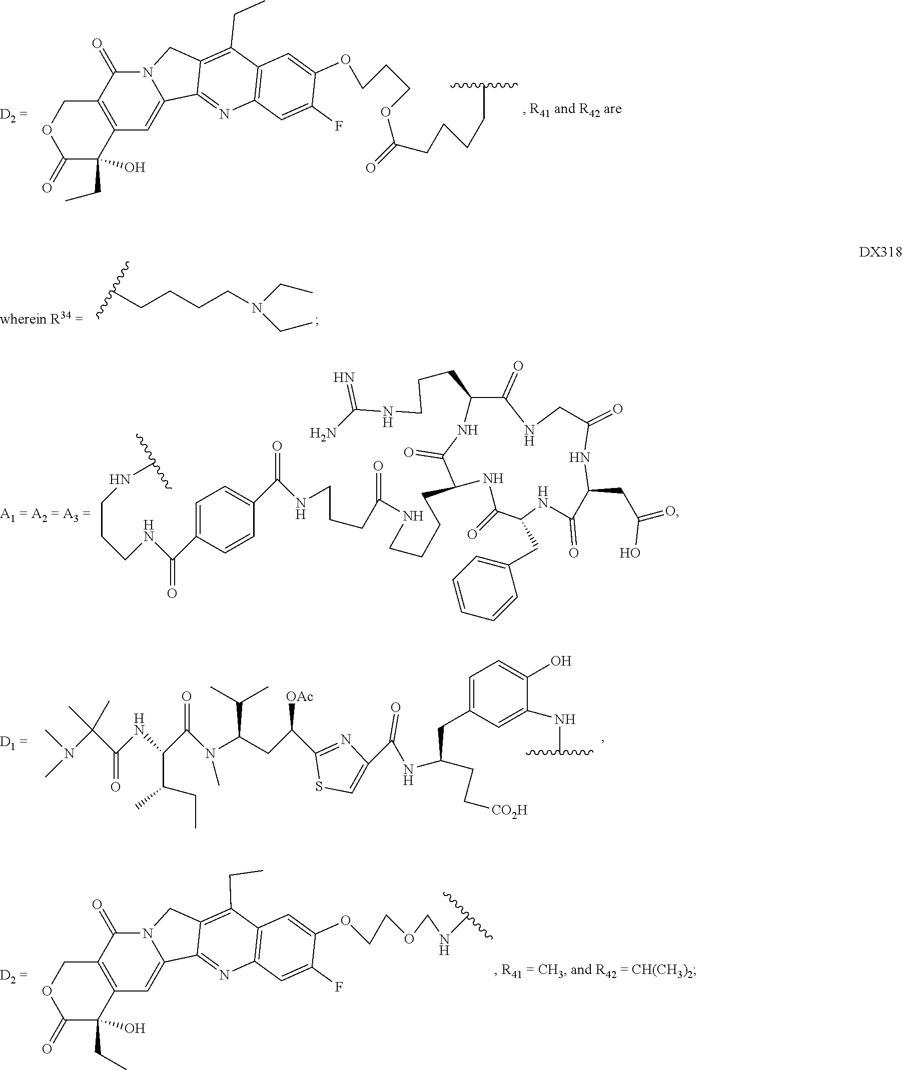

wherein mAb above is an antibody; n is 1-30, preferably 1-20, more preferably 2~8.

In certain embodiments, the conjugates of Formula (I), (II) and (III) are prepared readily via conjugation reaction of the antibody with compounds having the following formula (V), (VI) and (VII) respectively:

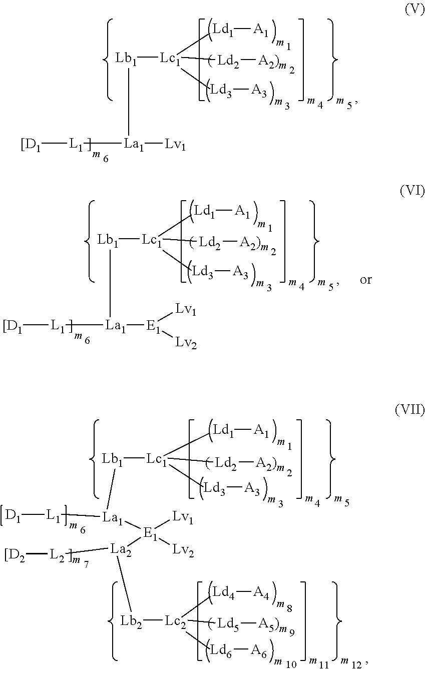

Or the conjugate of Formula (IV) is prepared through sequential conjugation reaction of Formula (V) and Formula (V′) to an antibody:

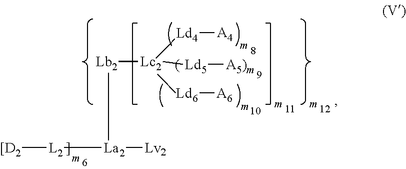

Wherein D₁, D₂, L₁, L₂, La₁, La₂, Lb₁, Lb₂, Le₁, Lc₂, Ld₁, Ld₂, Ld₃, Ld₄, Ld₅, Ld₆, A₁, A₂, A₃, A₄, A₅, A₆, E₁, m₁, m₂, m₃, ma, m₅, m₆, m₇, m₈, m₉, m₁₀, m₁₁, and m₁₂ are defined the same in the Formula (1), (II) and (III);

Lv₁ and Lv₂ are a reactive group and are independently or jointly selected from:

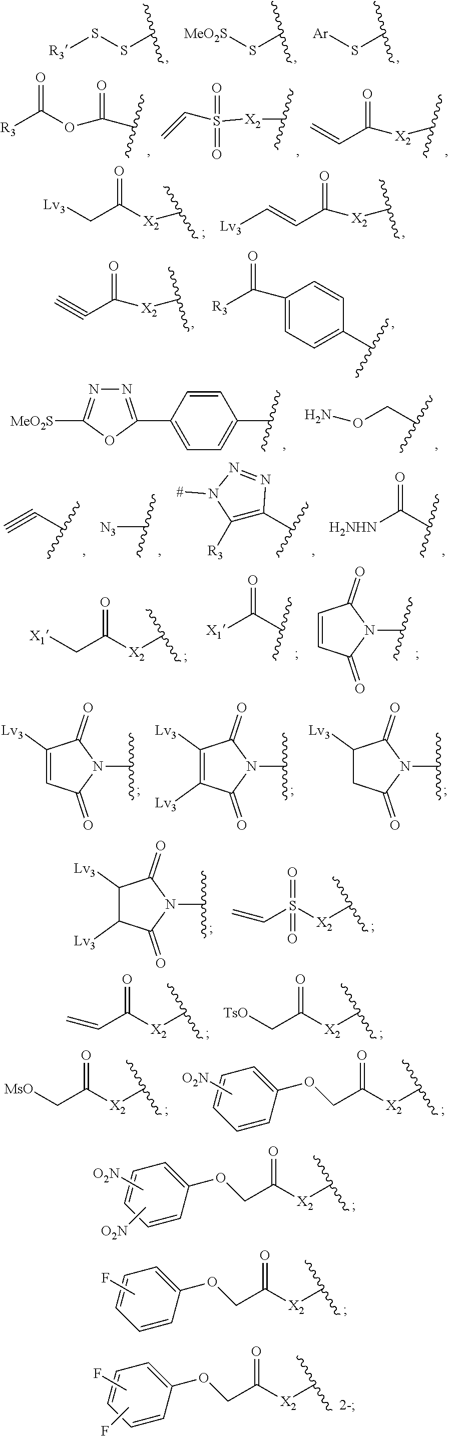

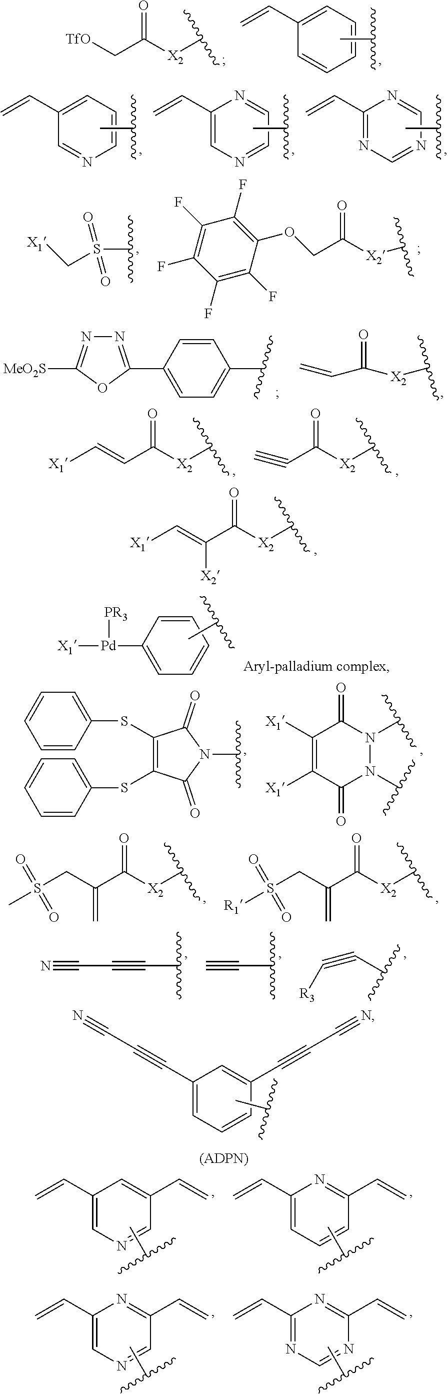

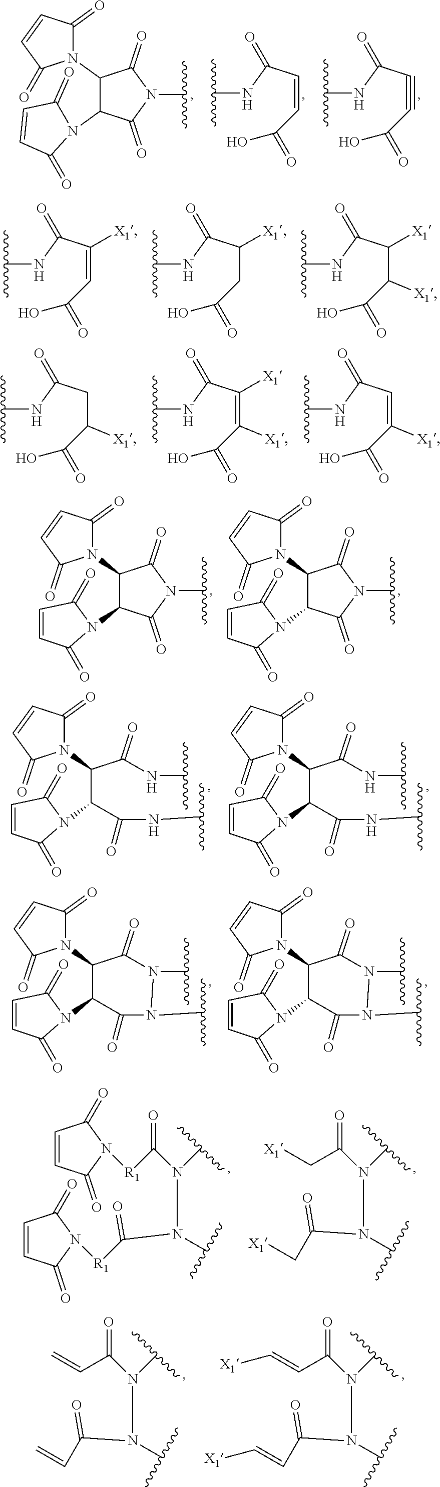

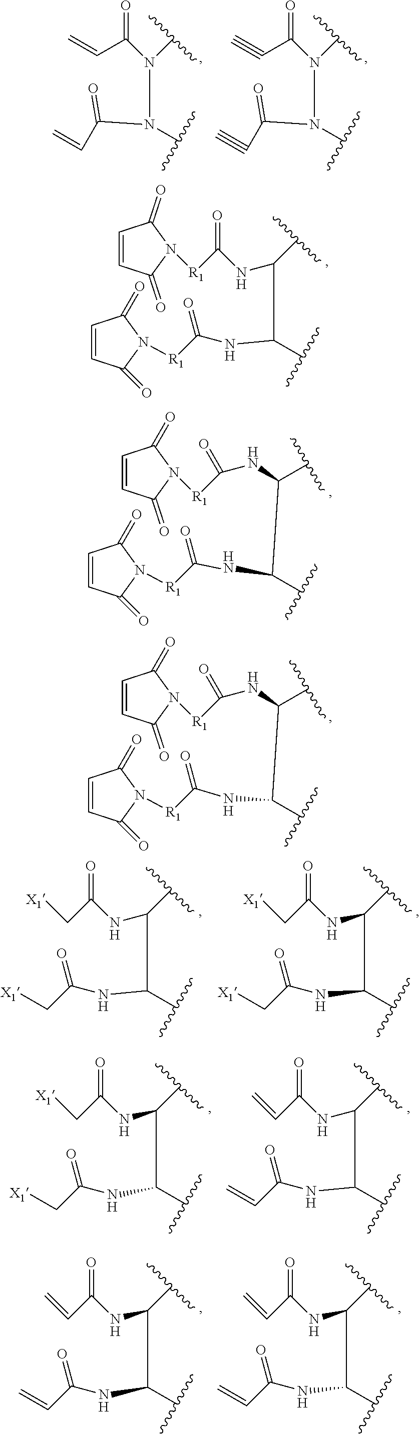

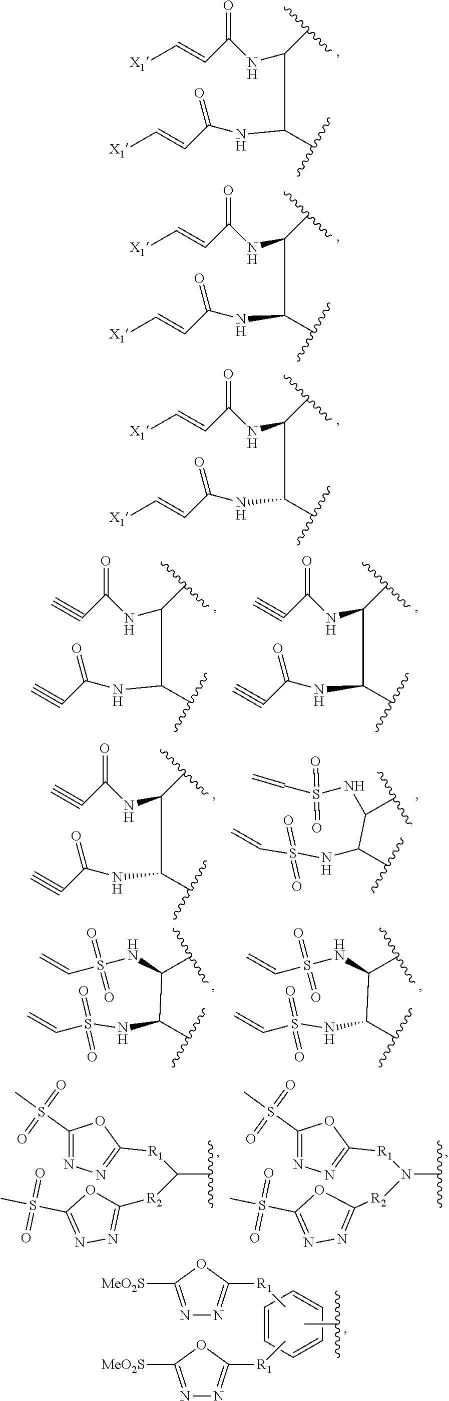

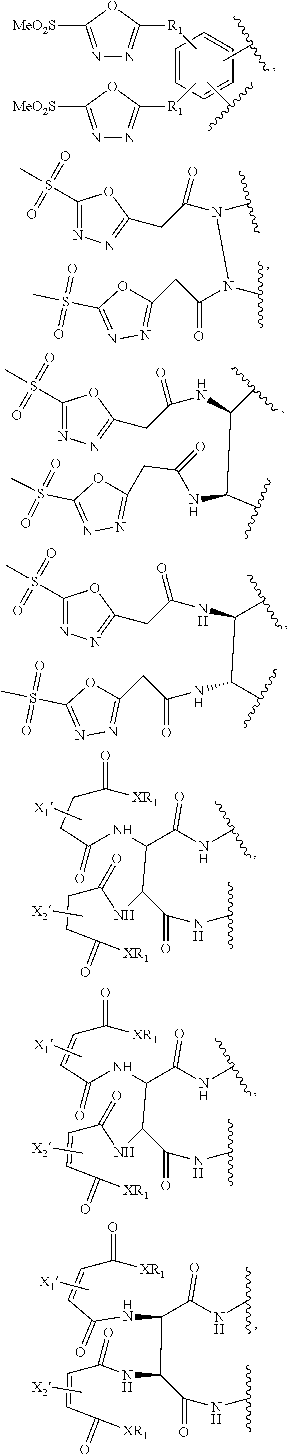

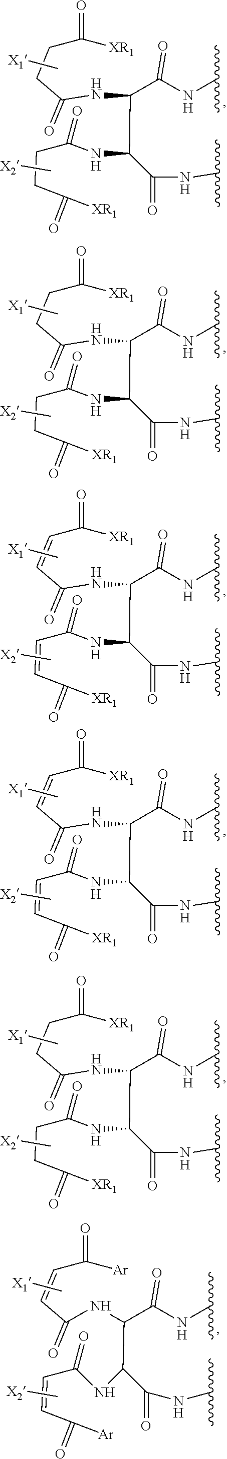

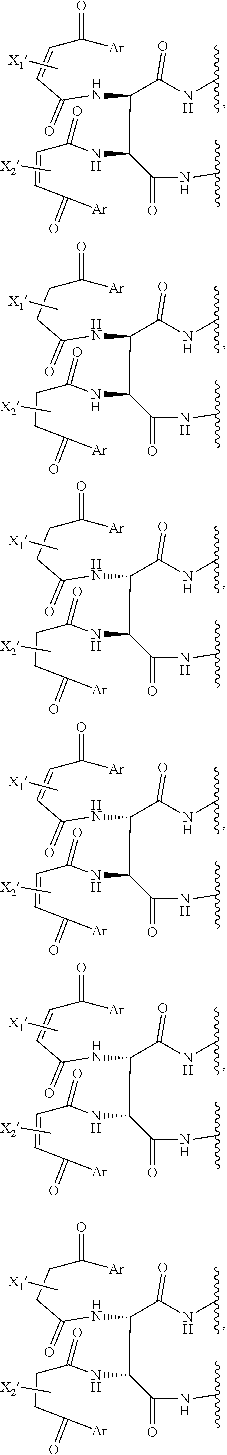

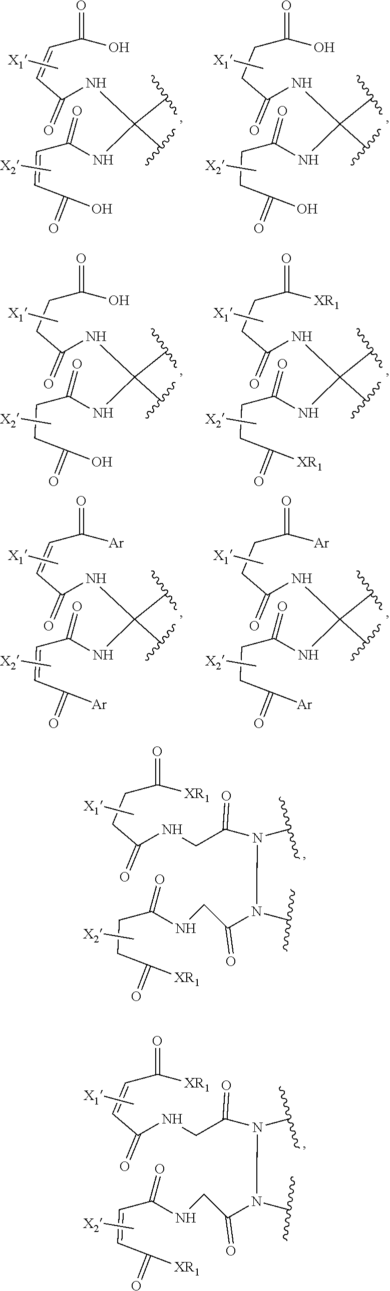

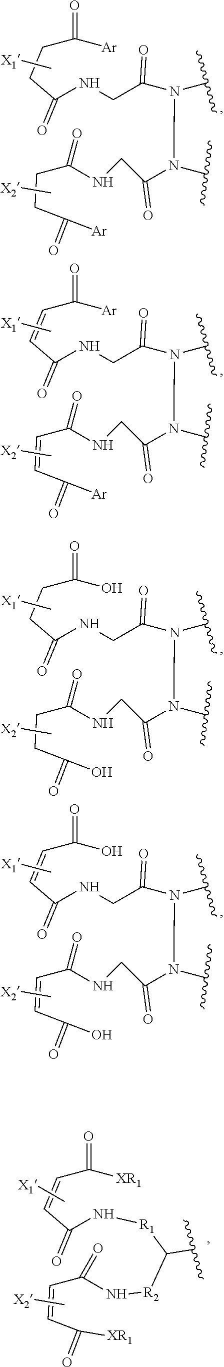

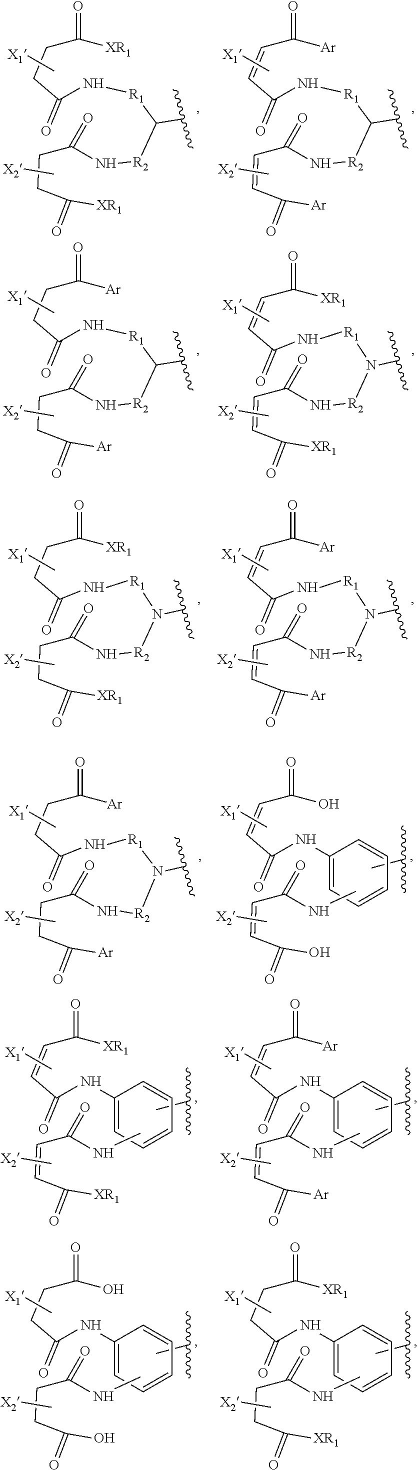

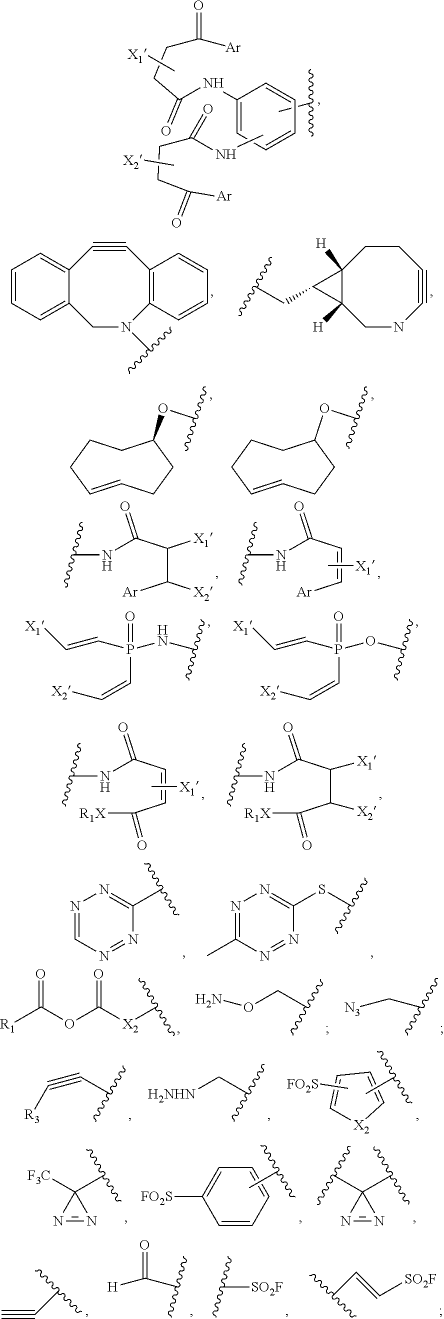

wherein X₁′ and X₂′ are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and Rs are independently H, R₁, aromatic, heteroaromatic, or aromatic group wherein one or several H atoms are replaced independently by —R₁, —halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

In the formula (VI) and formula (VII) wherein

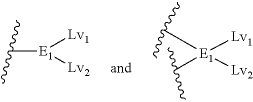

can be accordingly selected from:

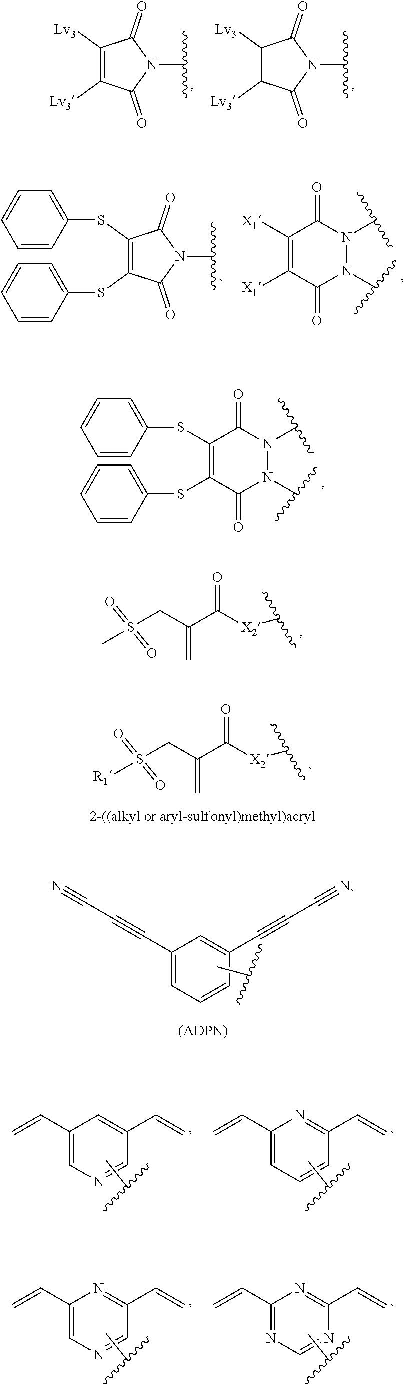

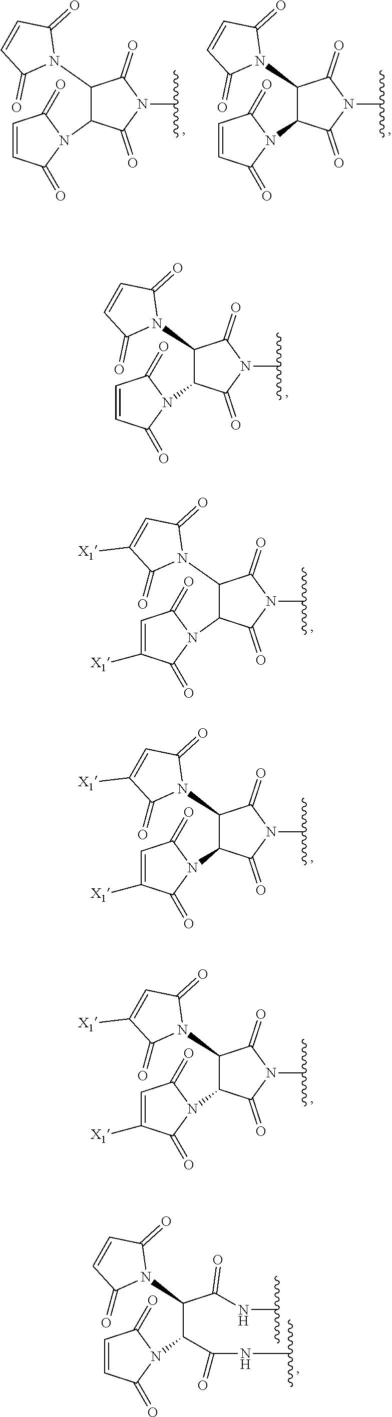

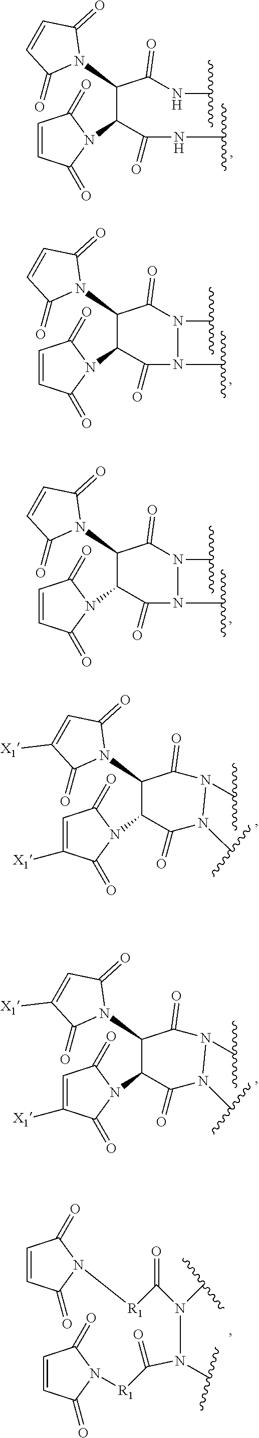

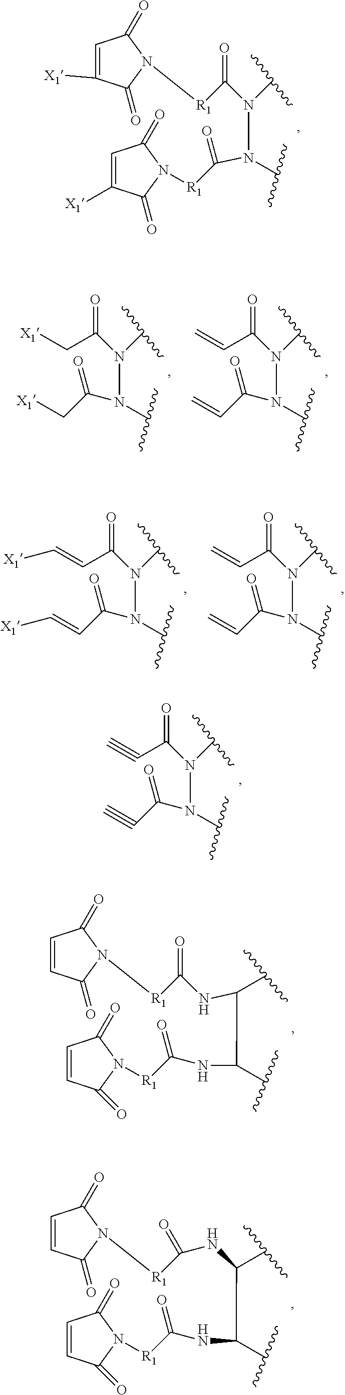

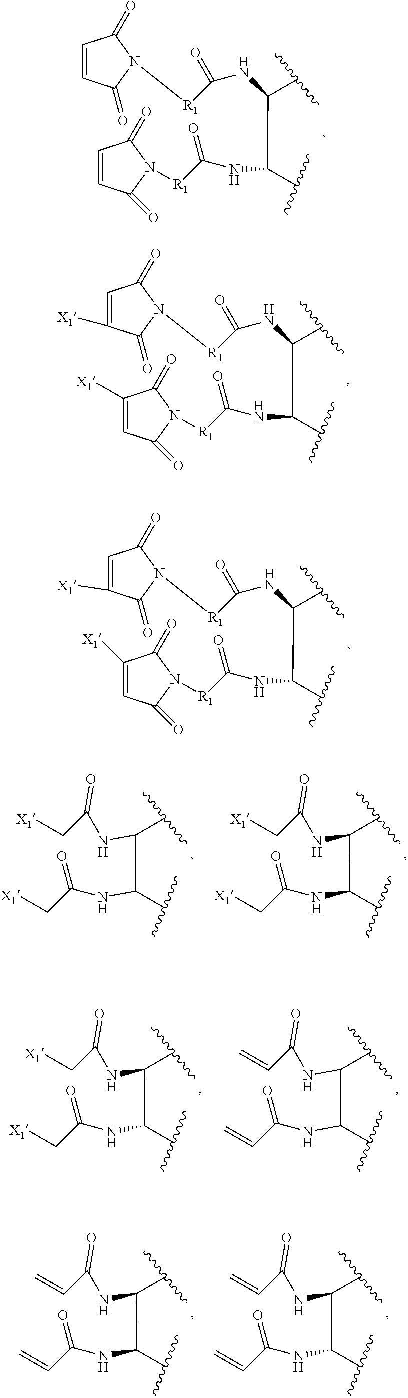

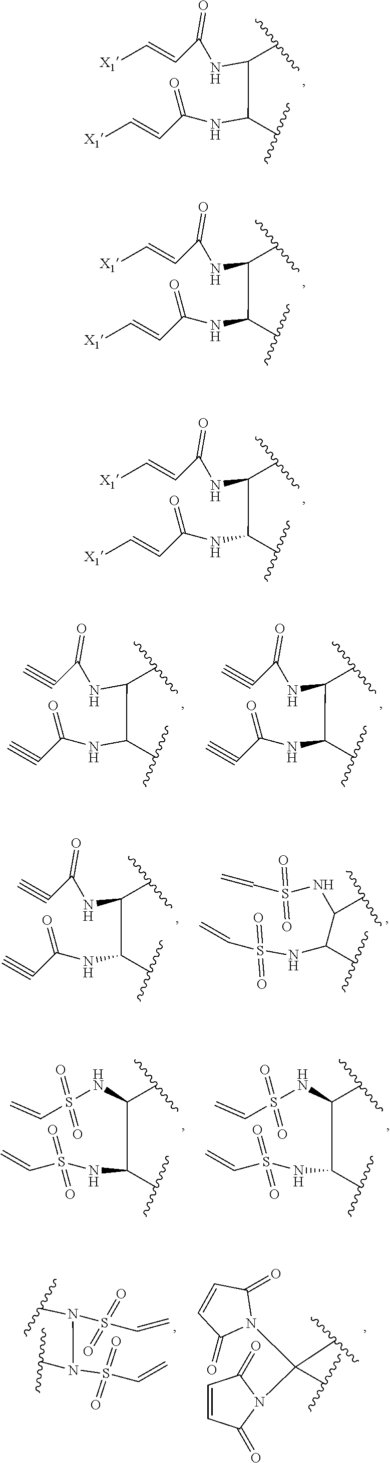

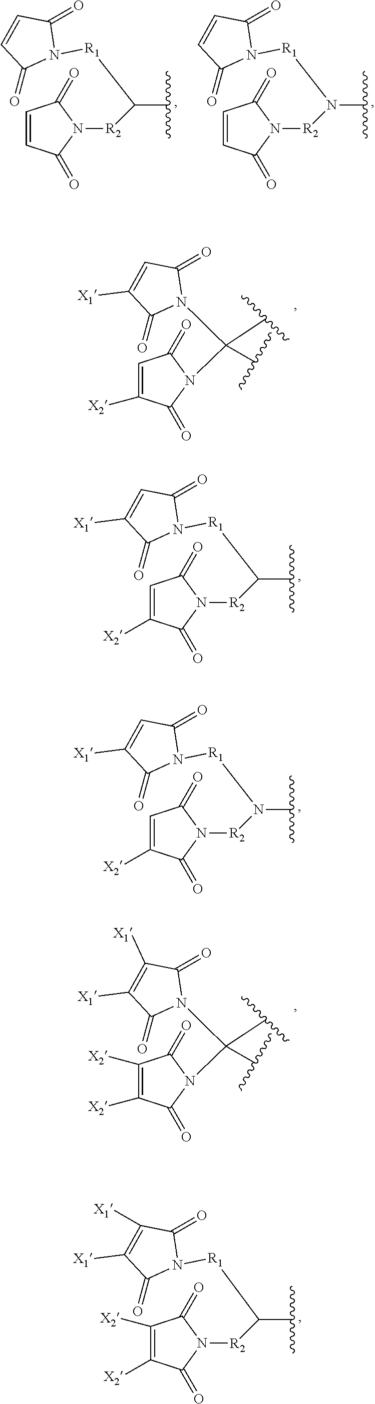

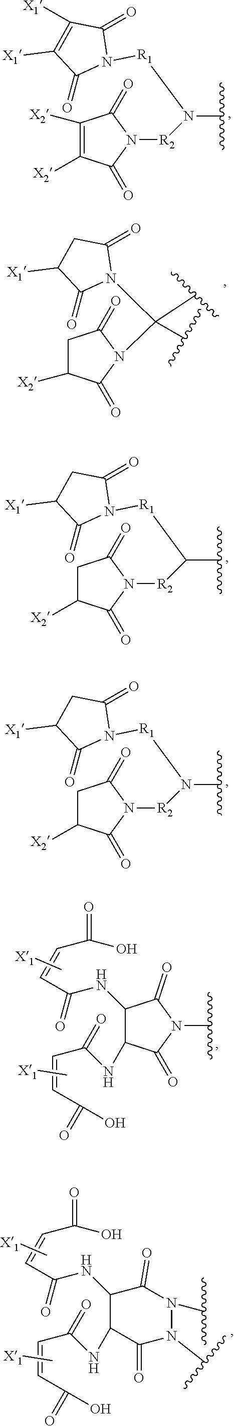

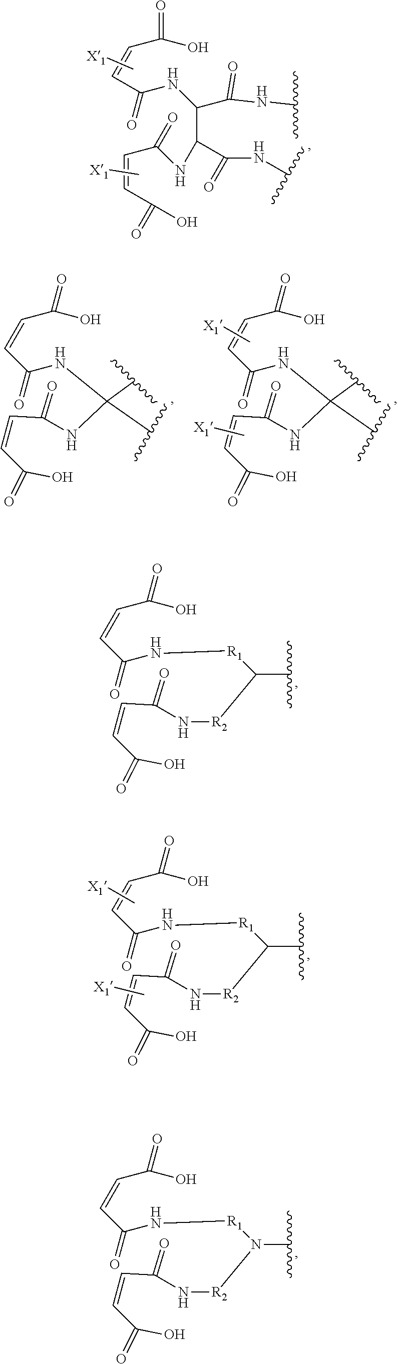

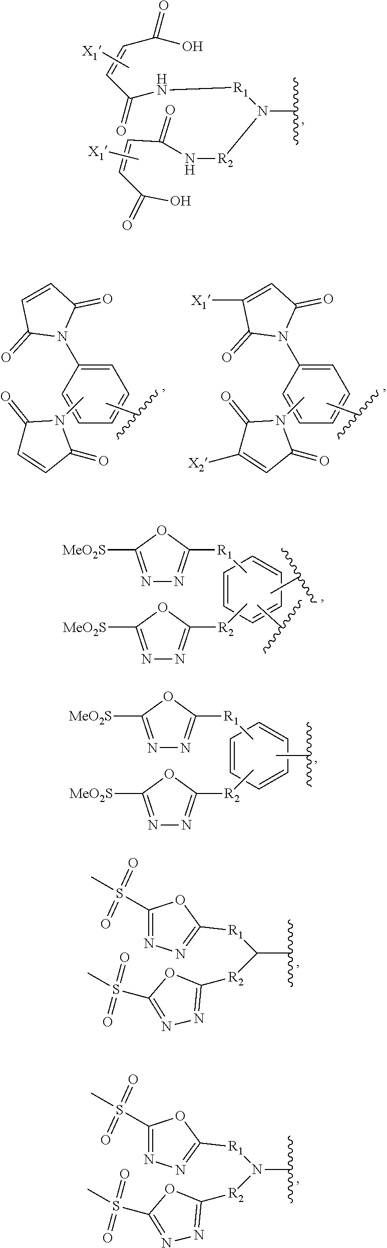

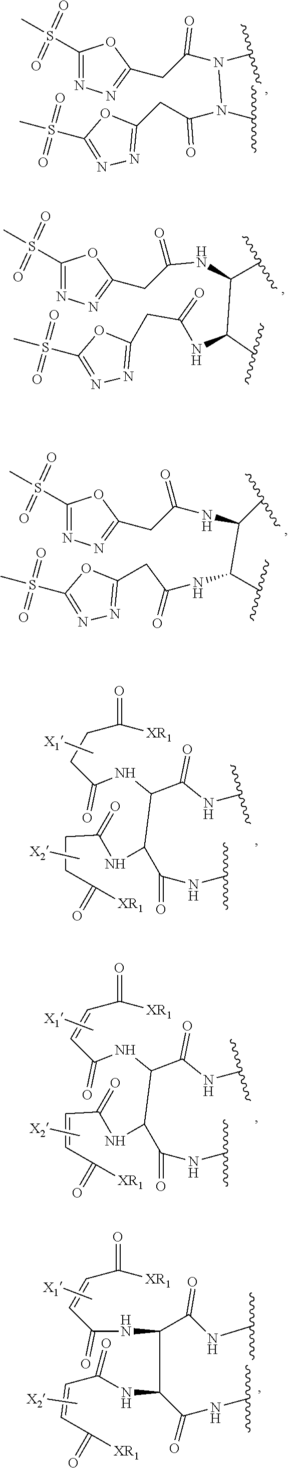

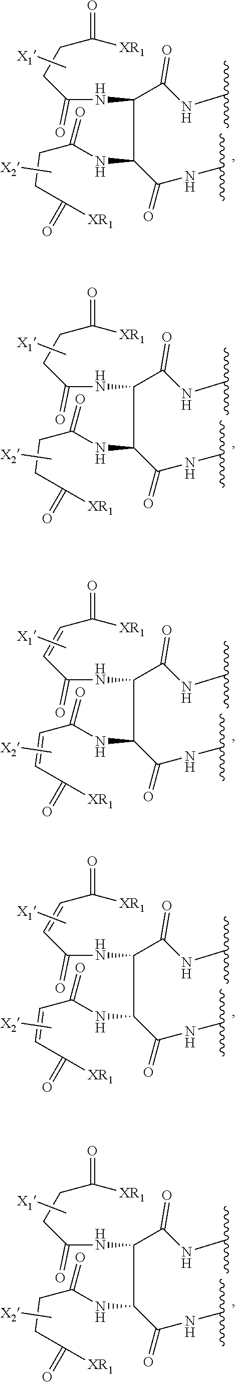

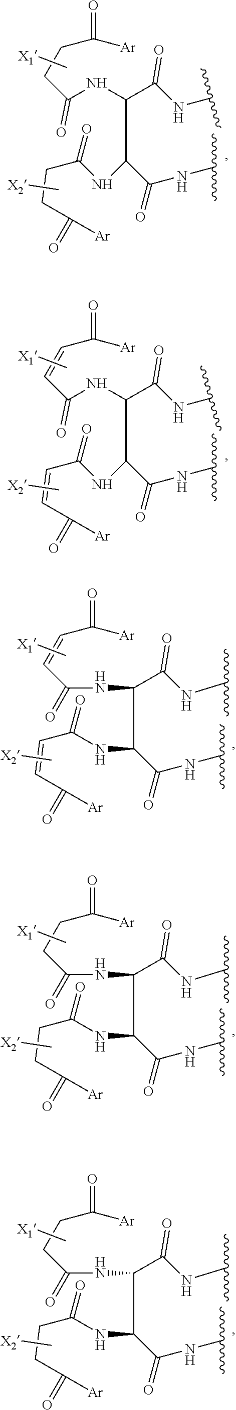

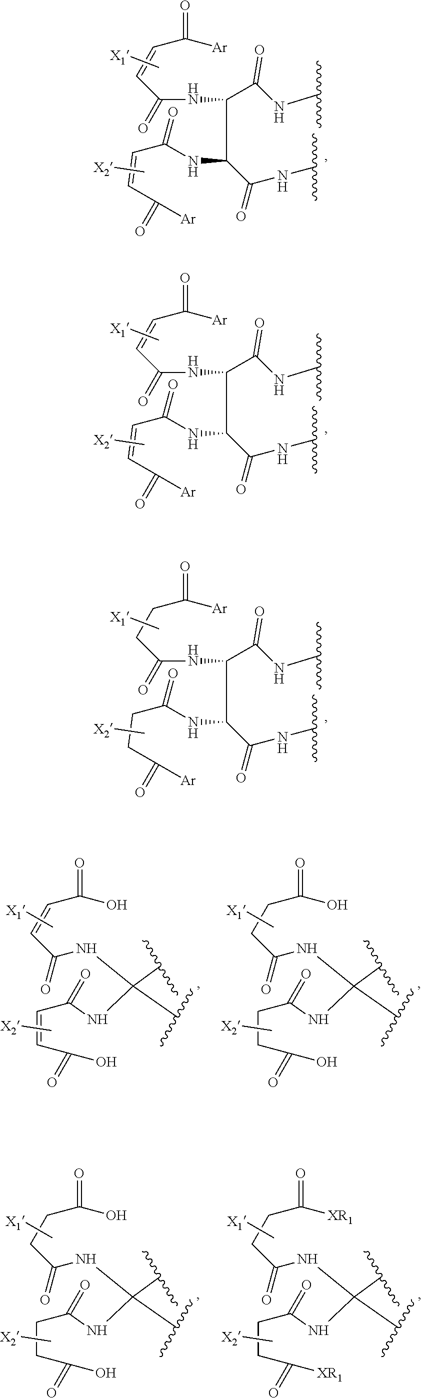

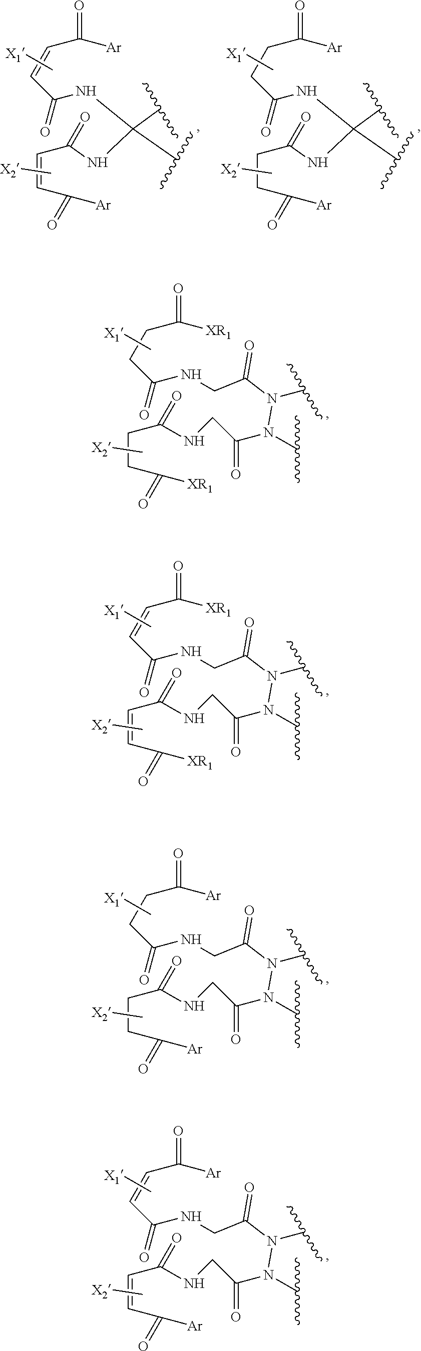

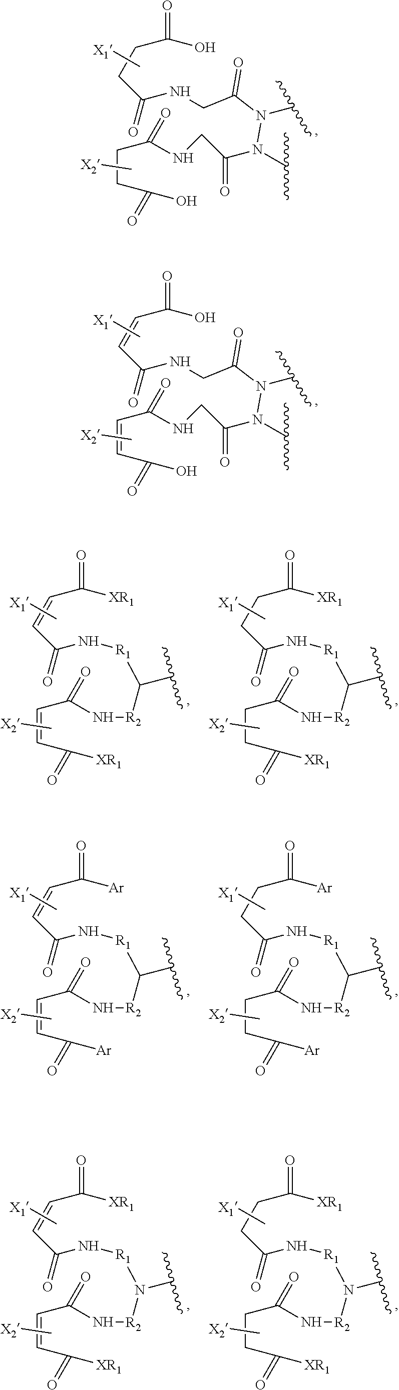

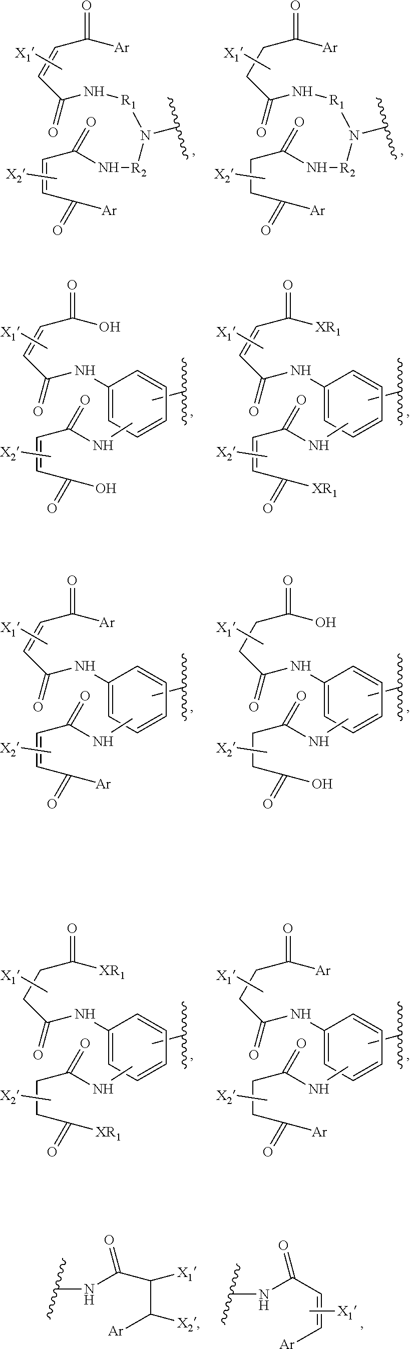

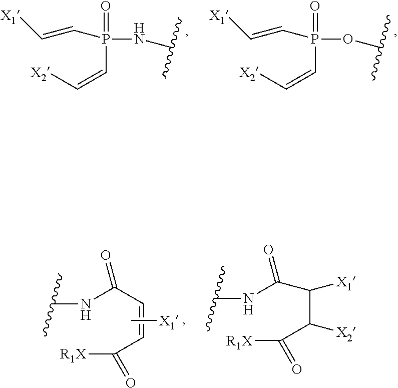

wherein Lv₃, Lv₃′, X₁′, and X₂′, are described above; the connecting bond “—” in the middle of the two atoms means it can link either one of the two atoms.

Examples of Formula (V), (VI), (VII) and (V′) are illustrated below:

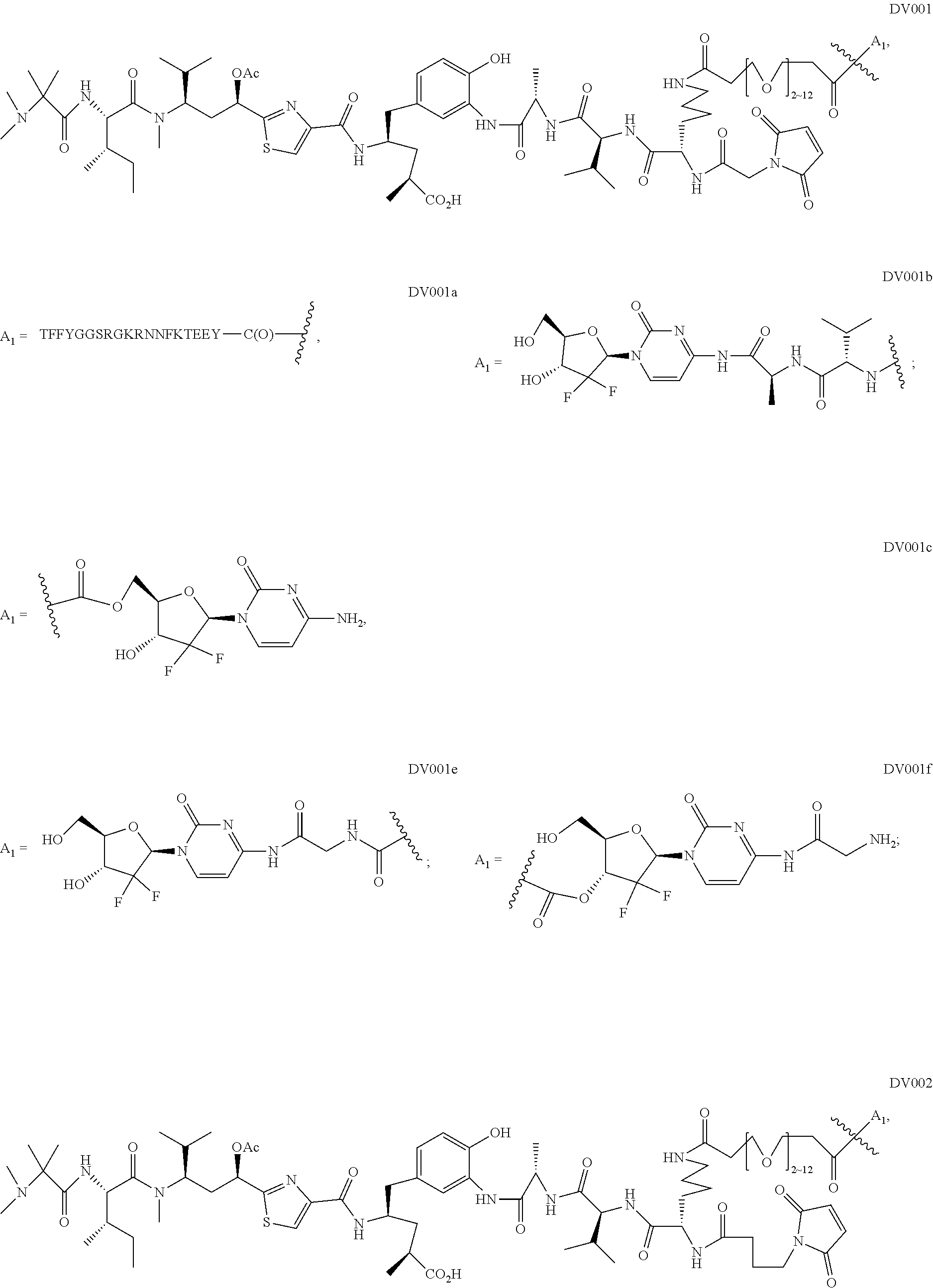

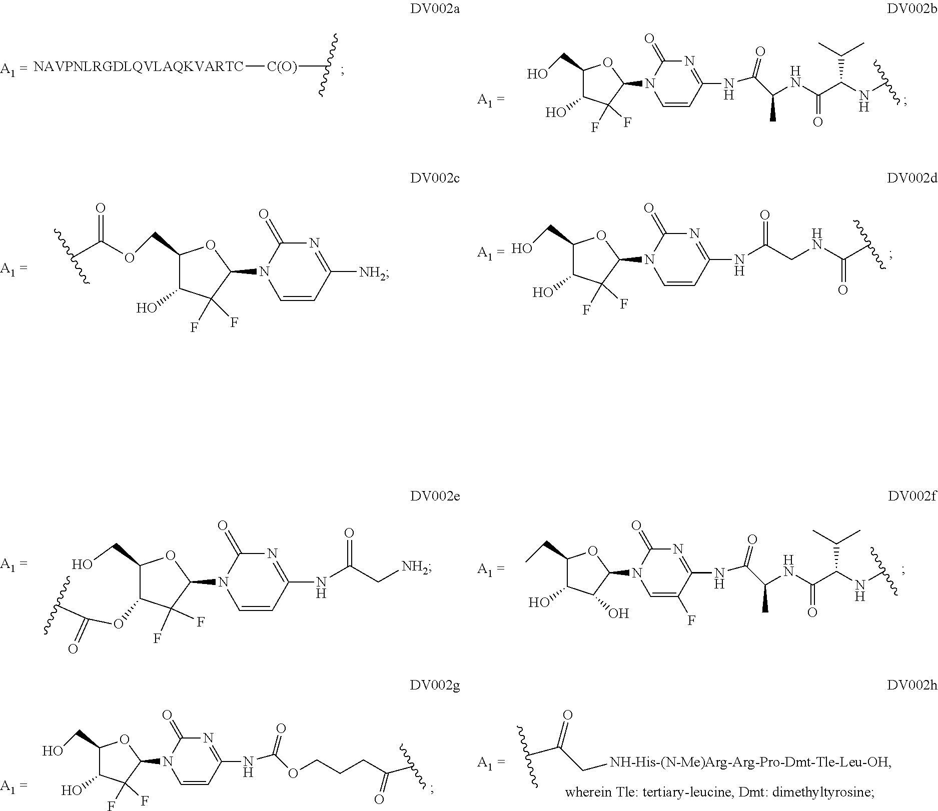

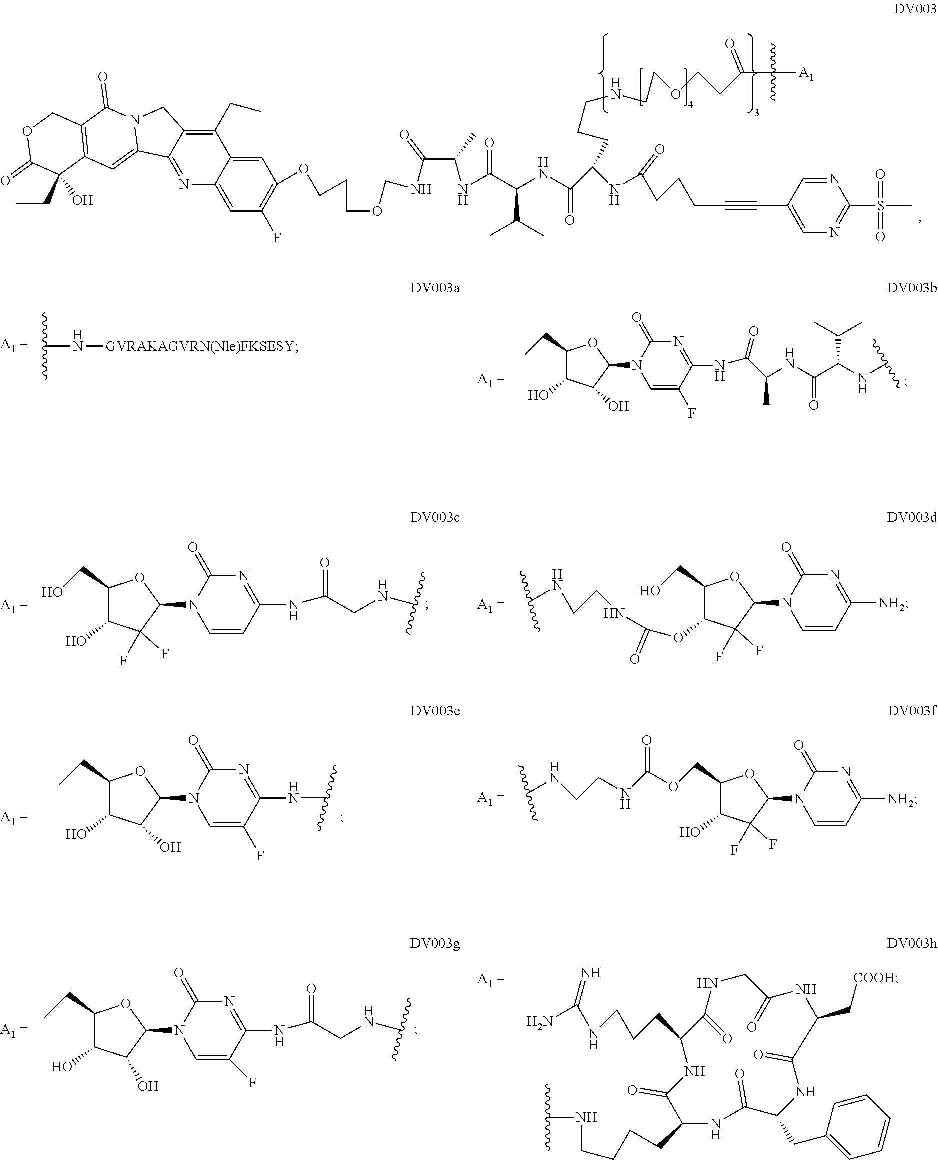

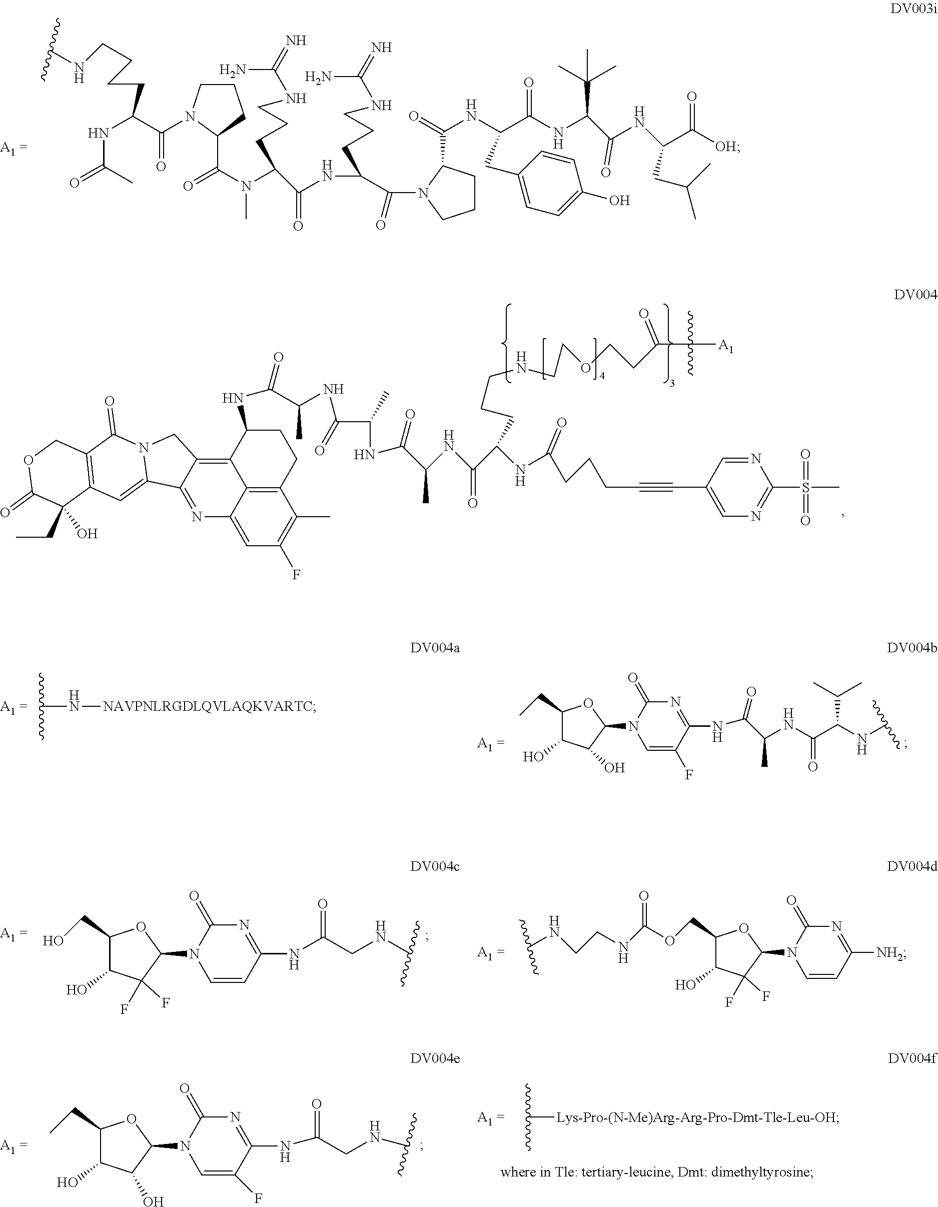

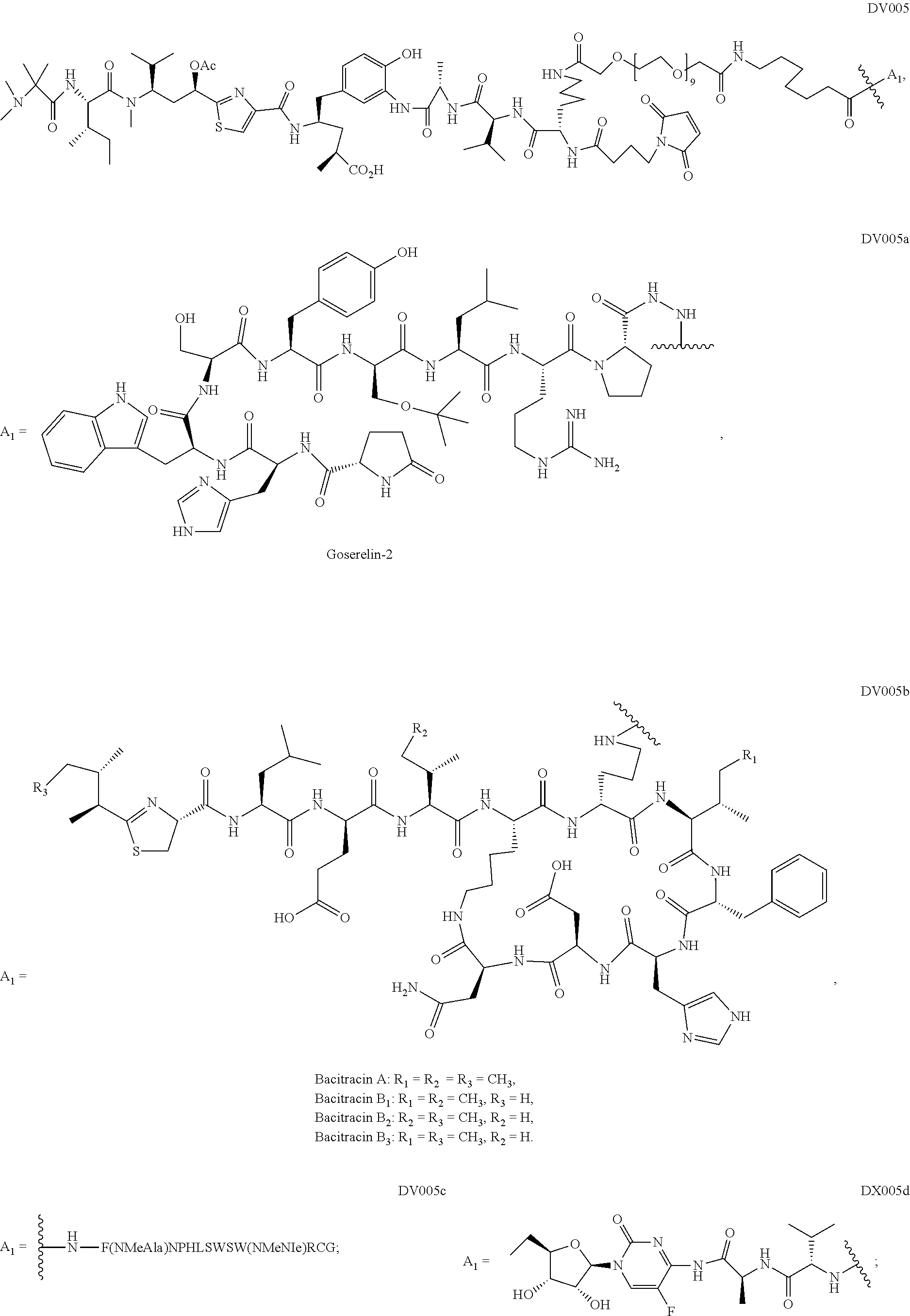

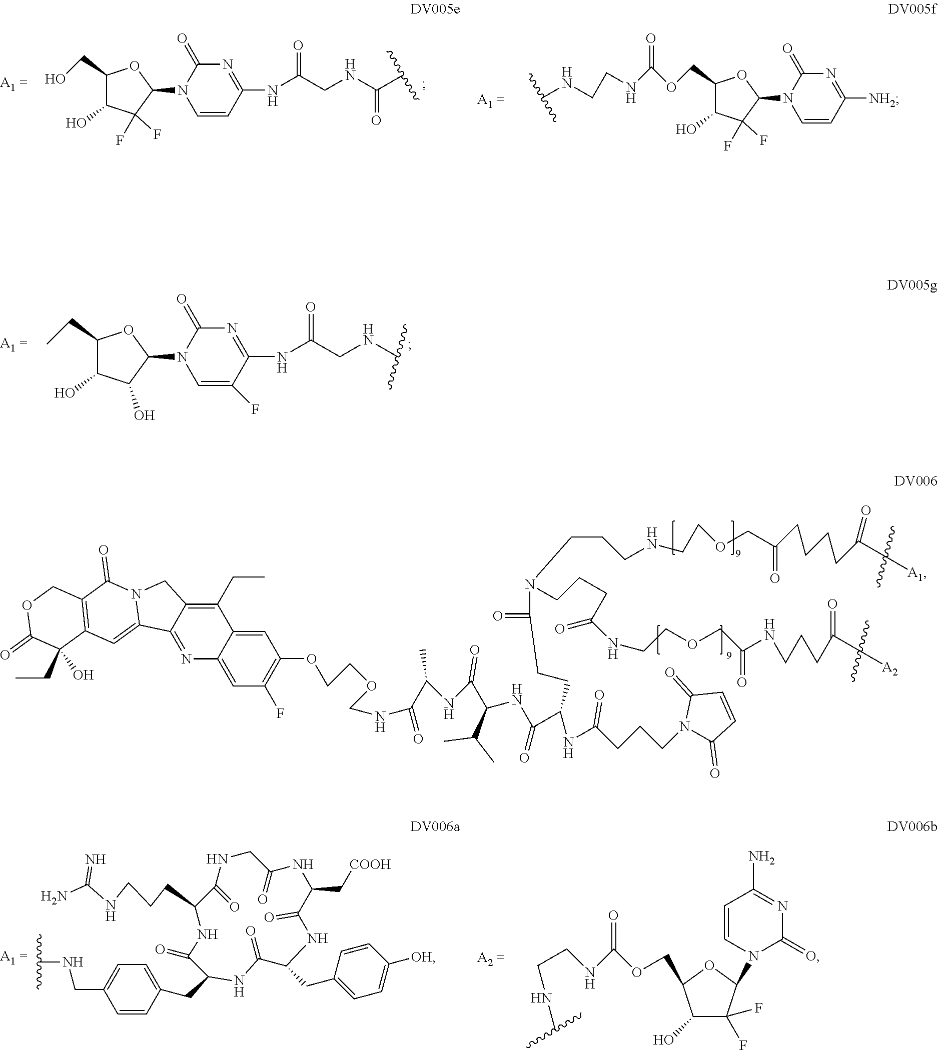

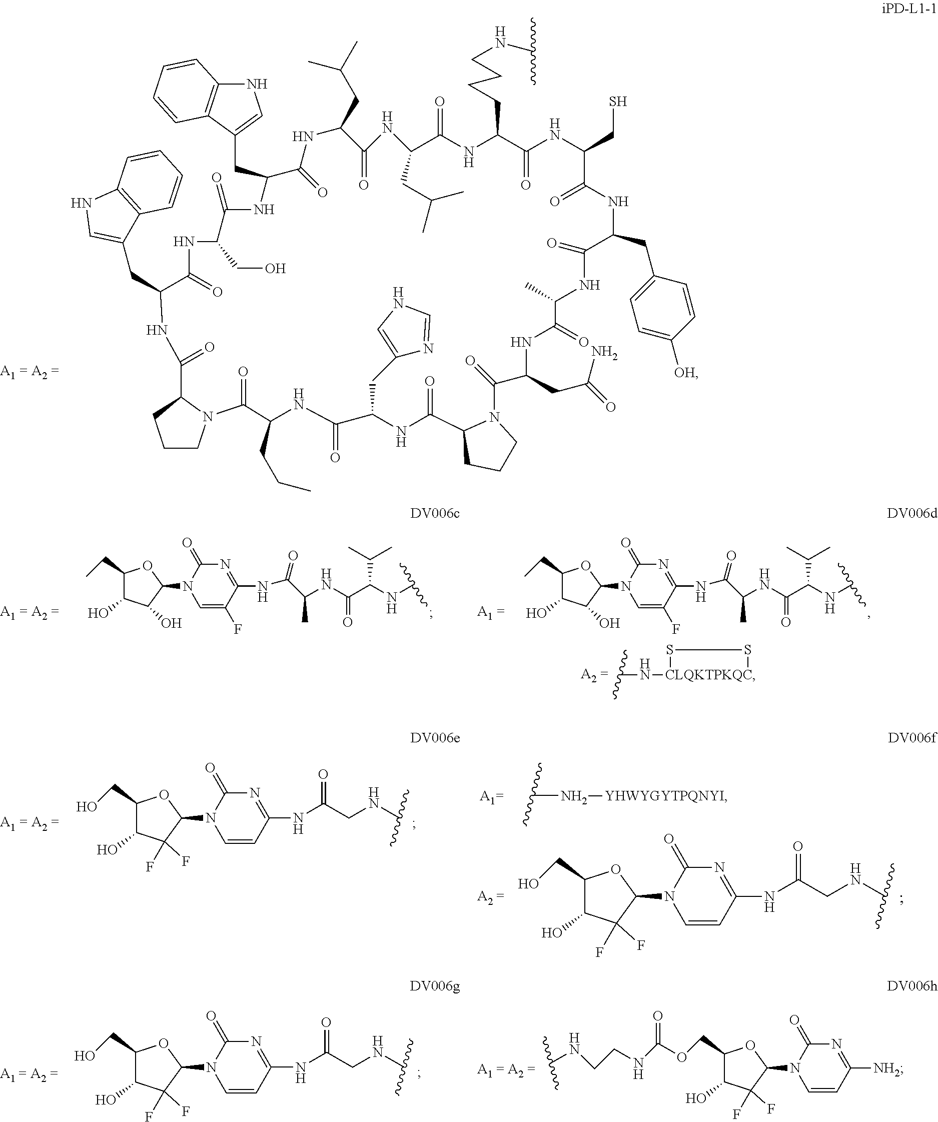

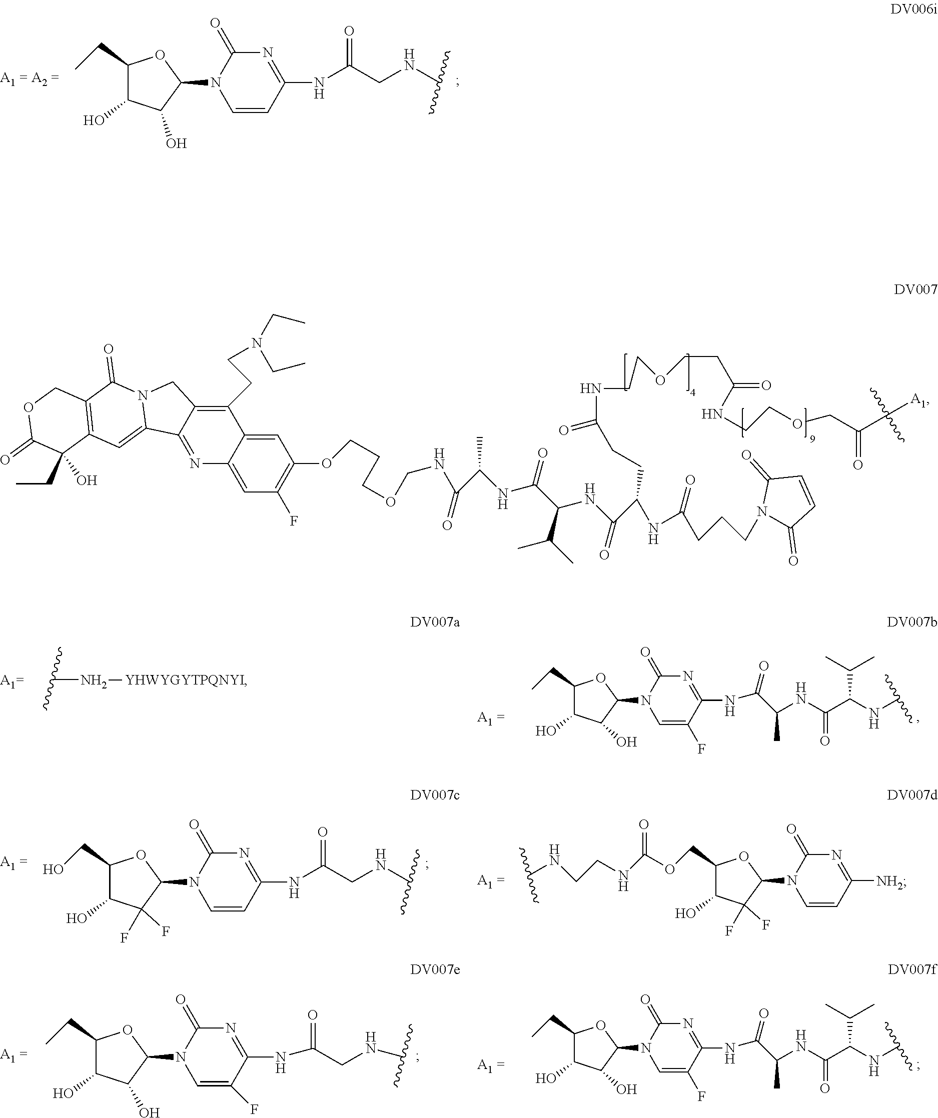

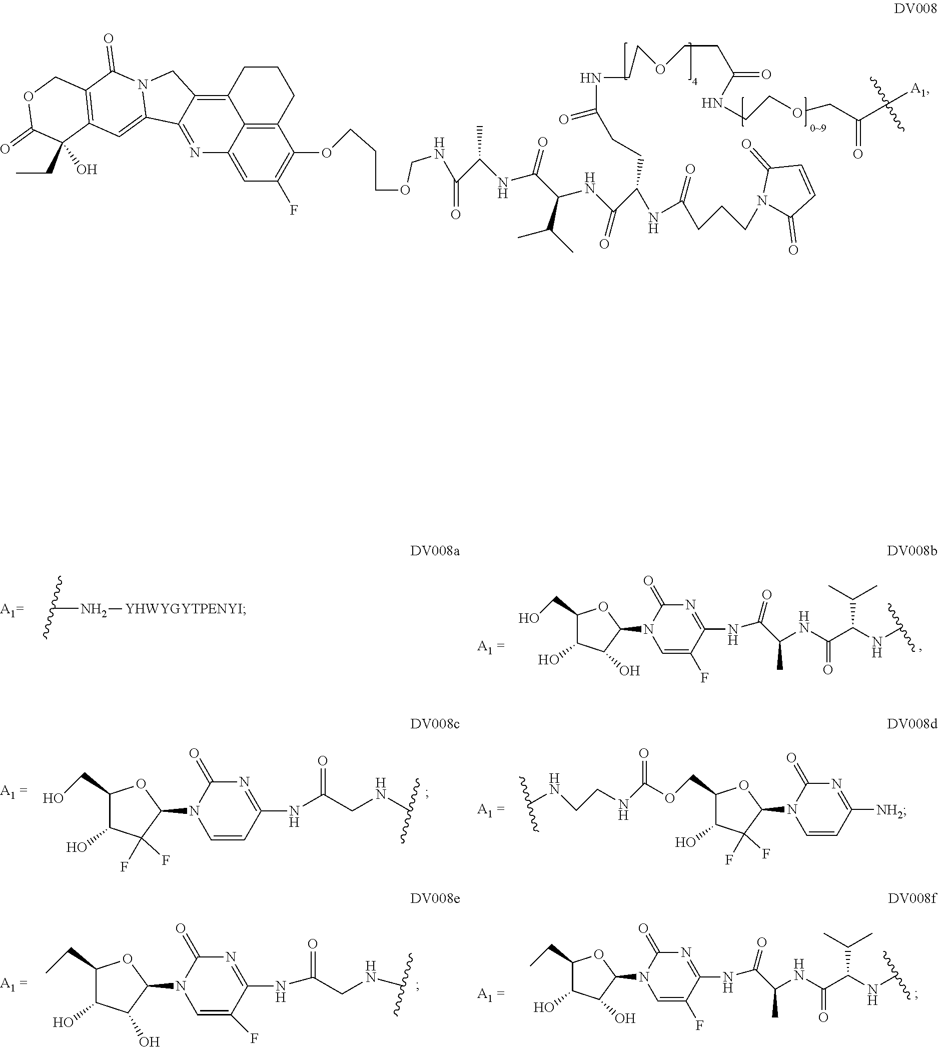

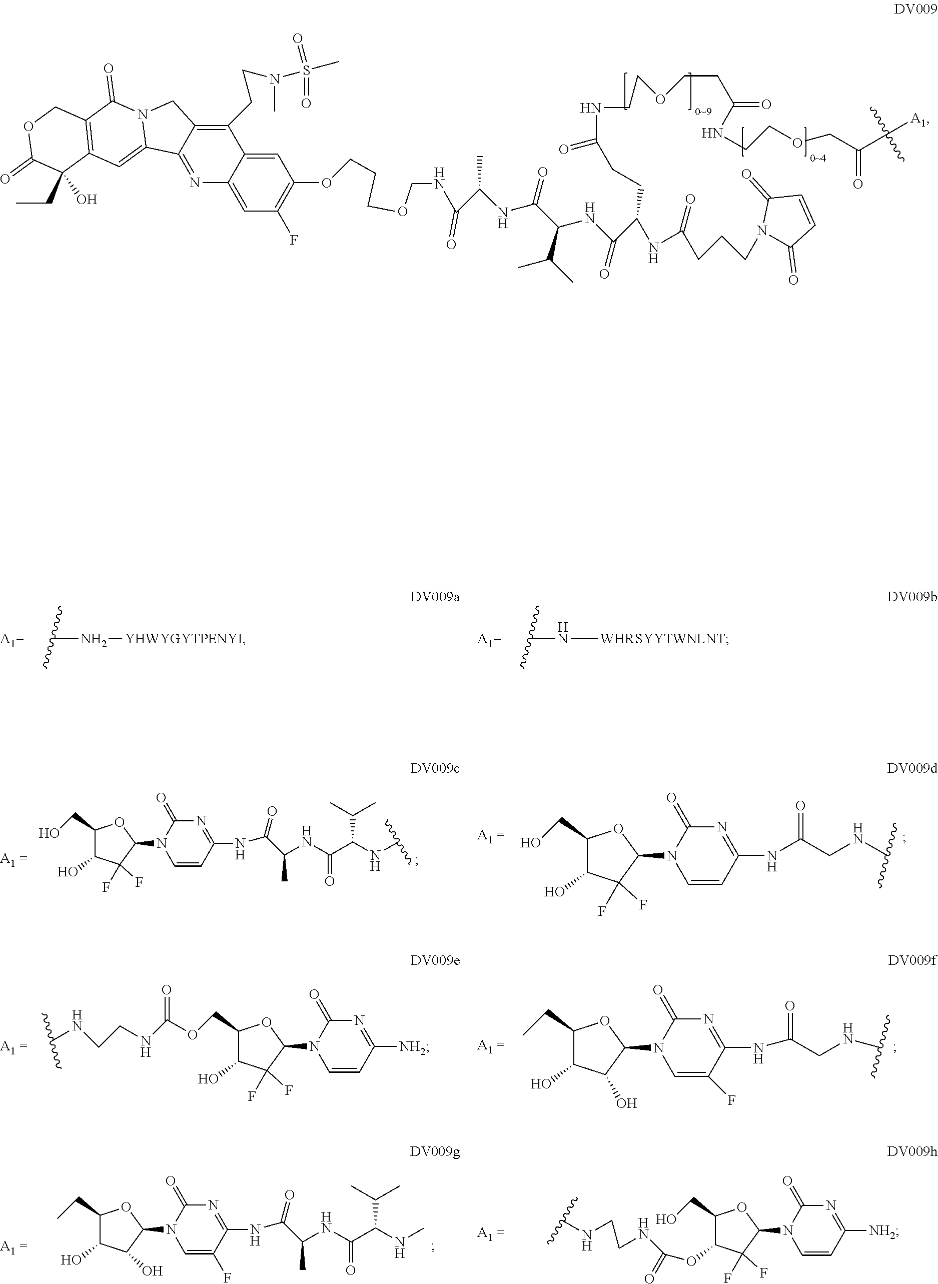

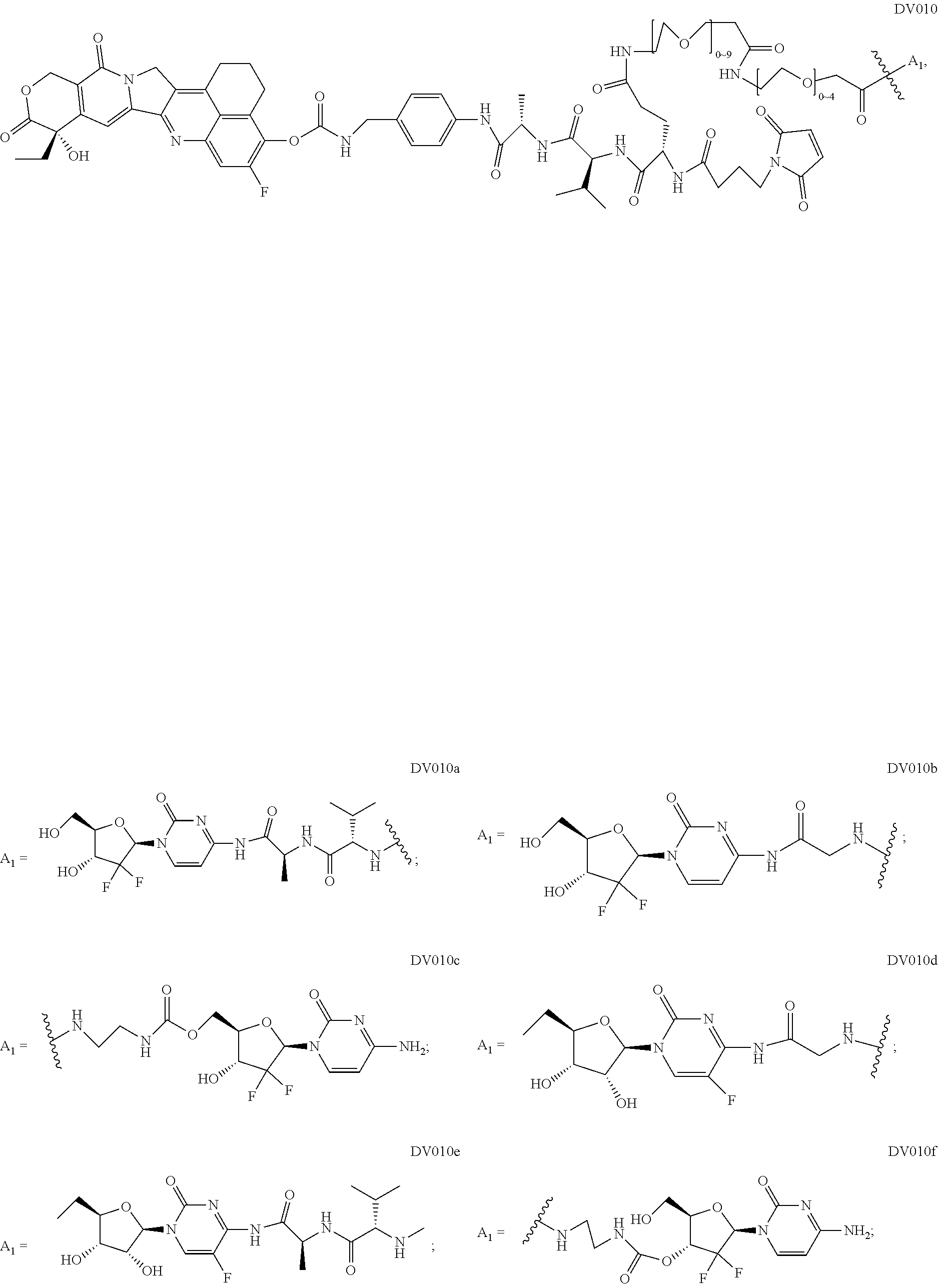

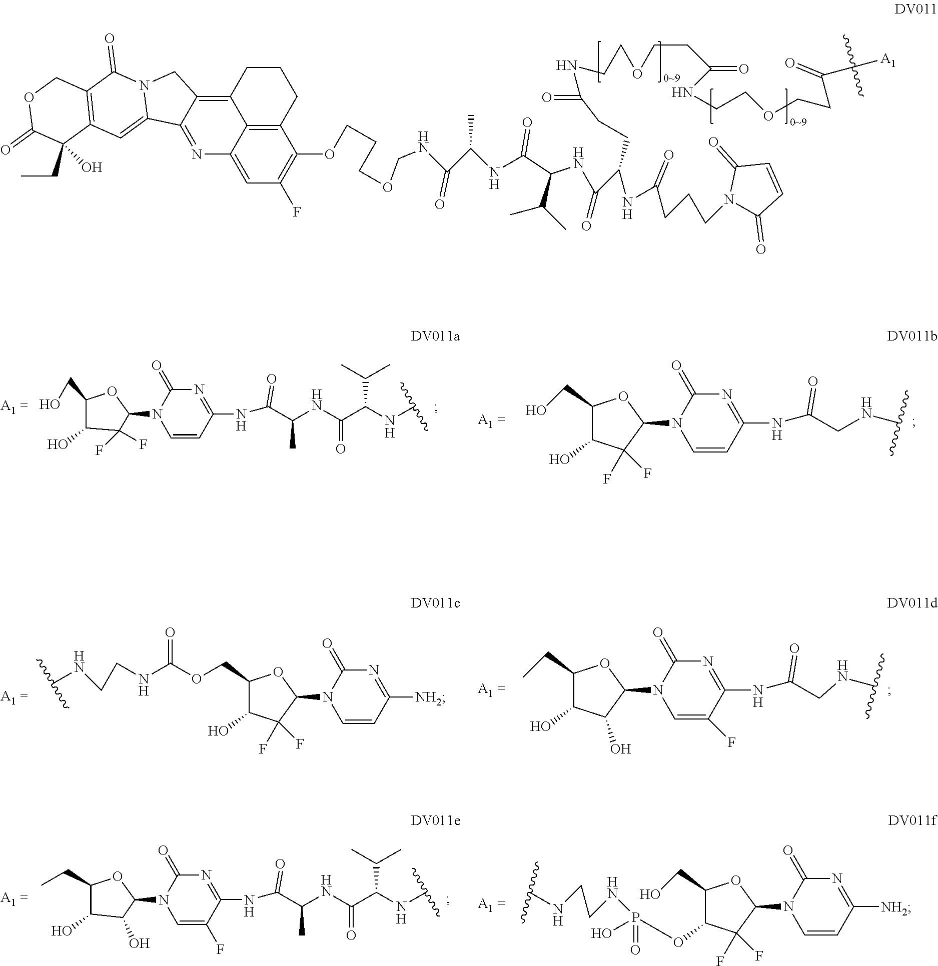

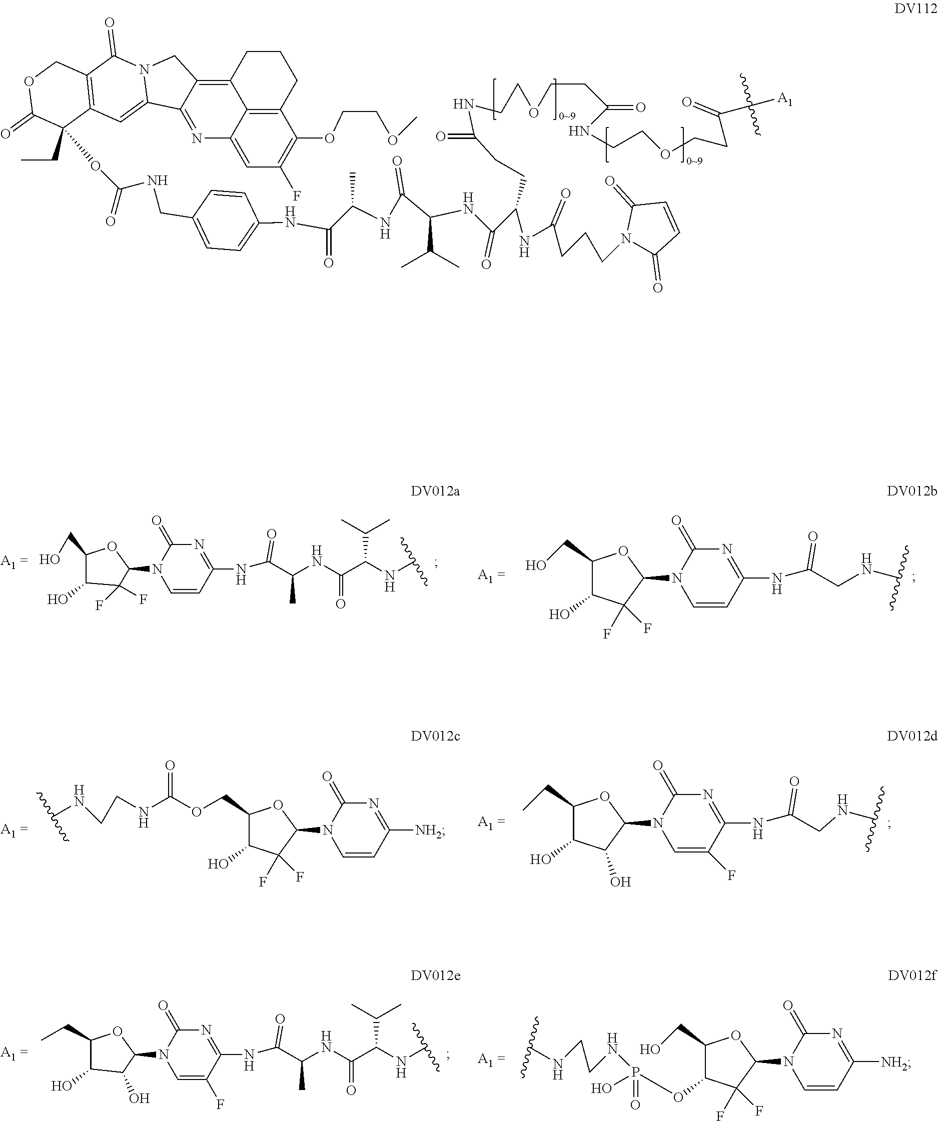

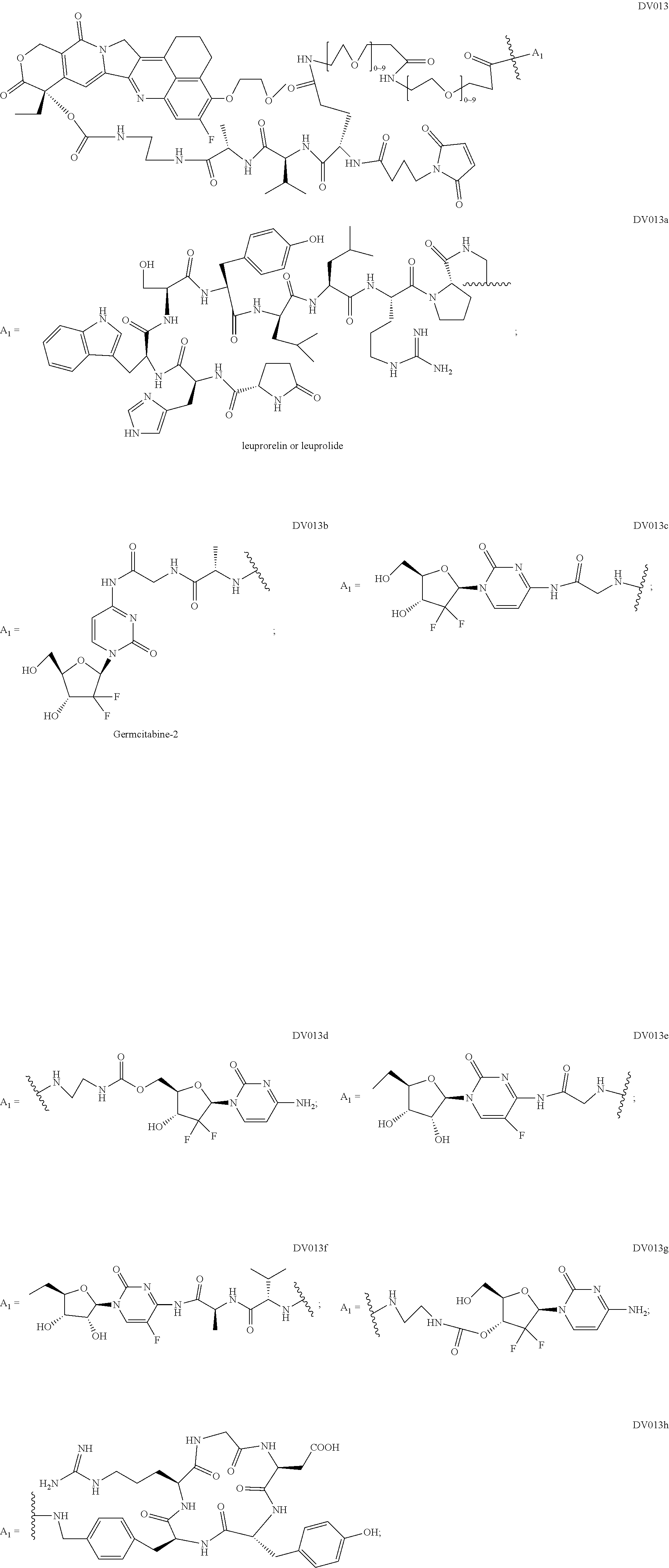

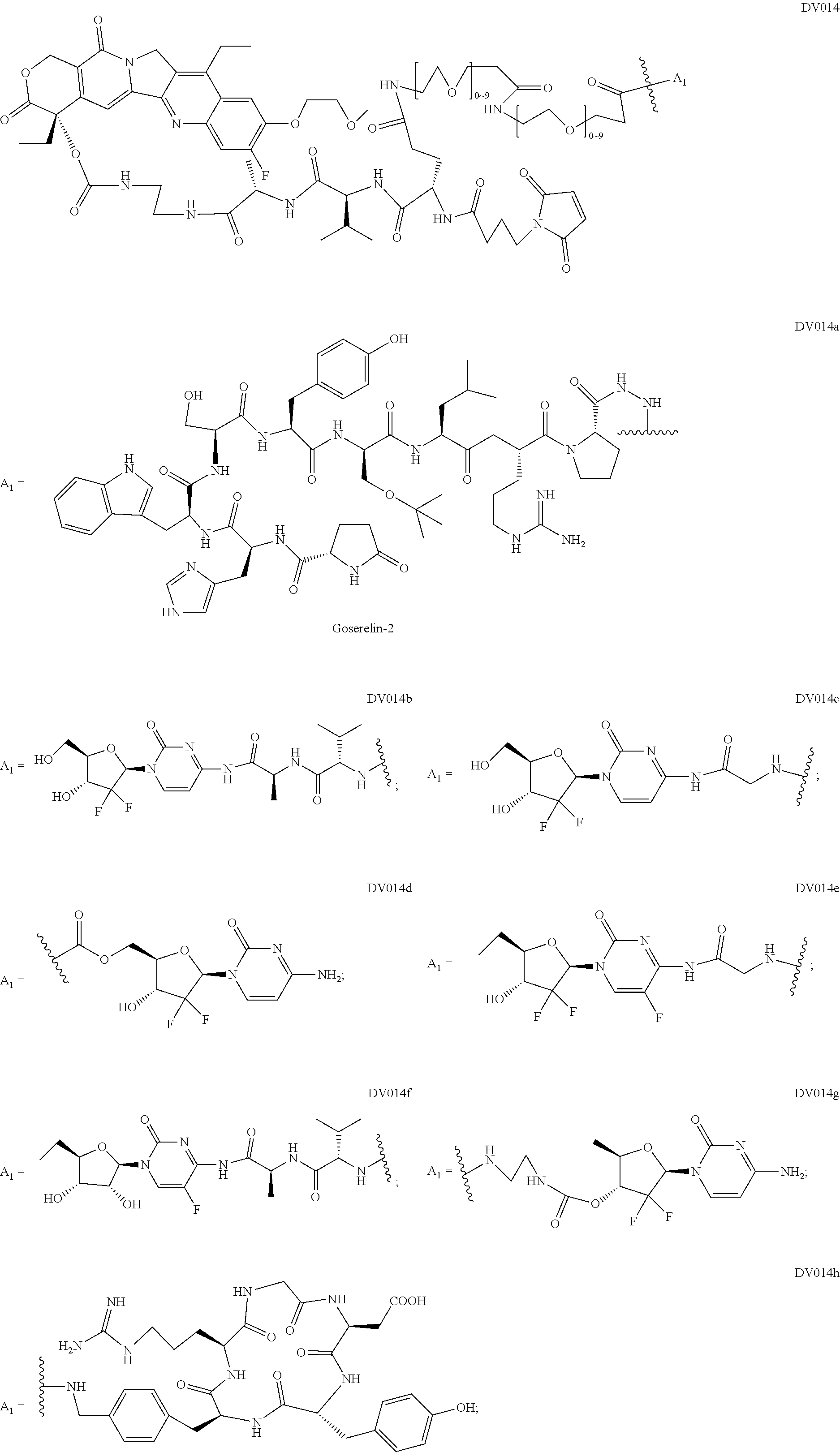

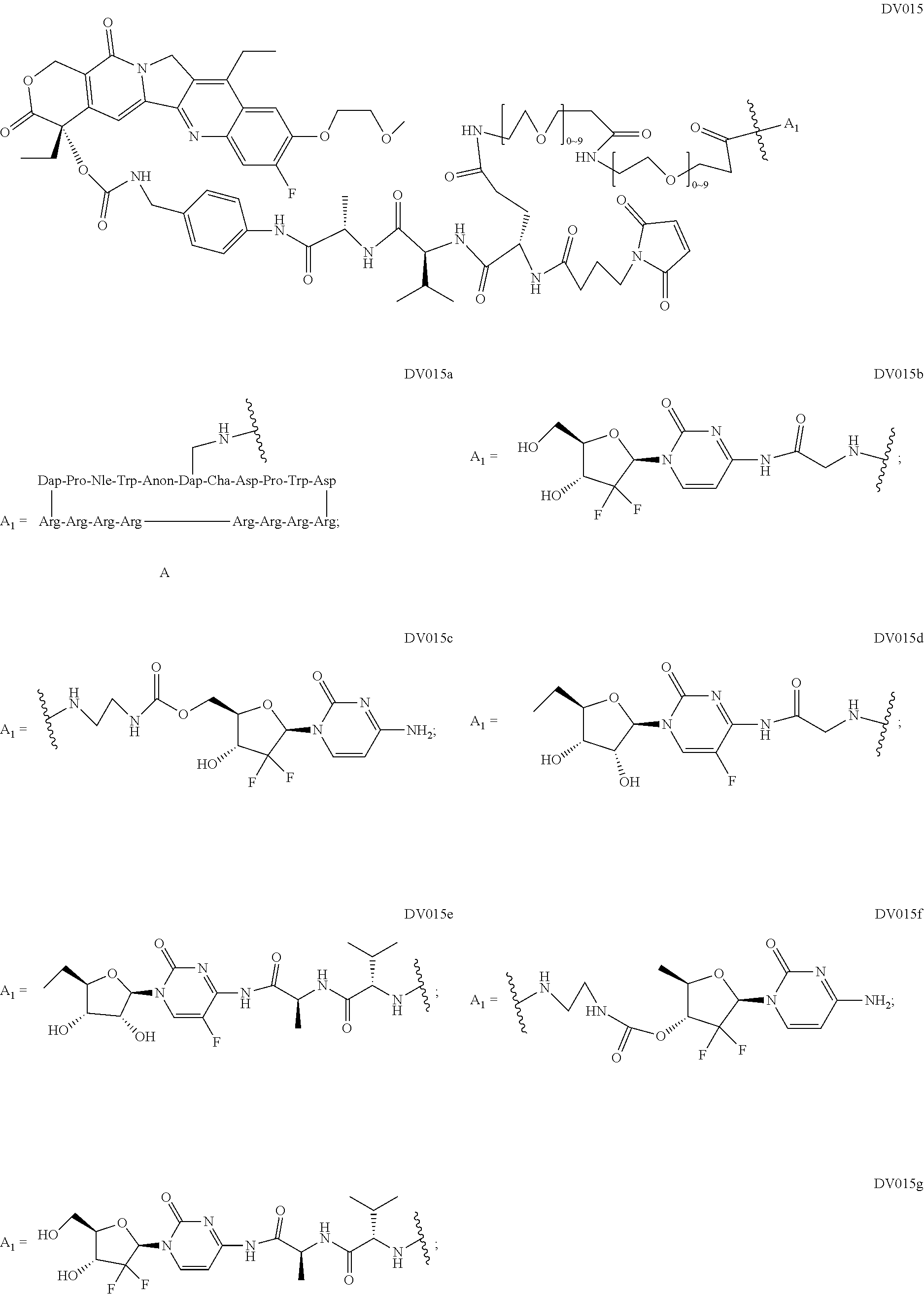

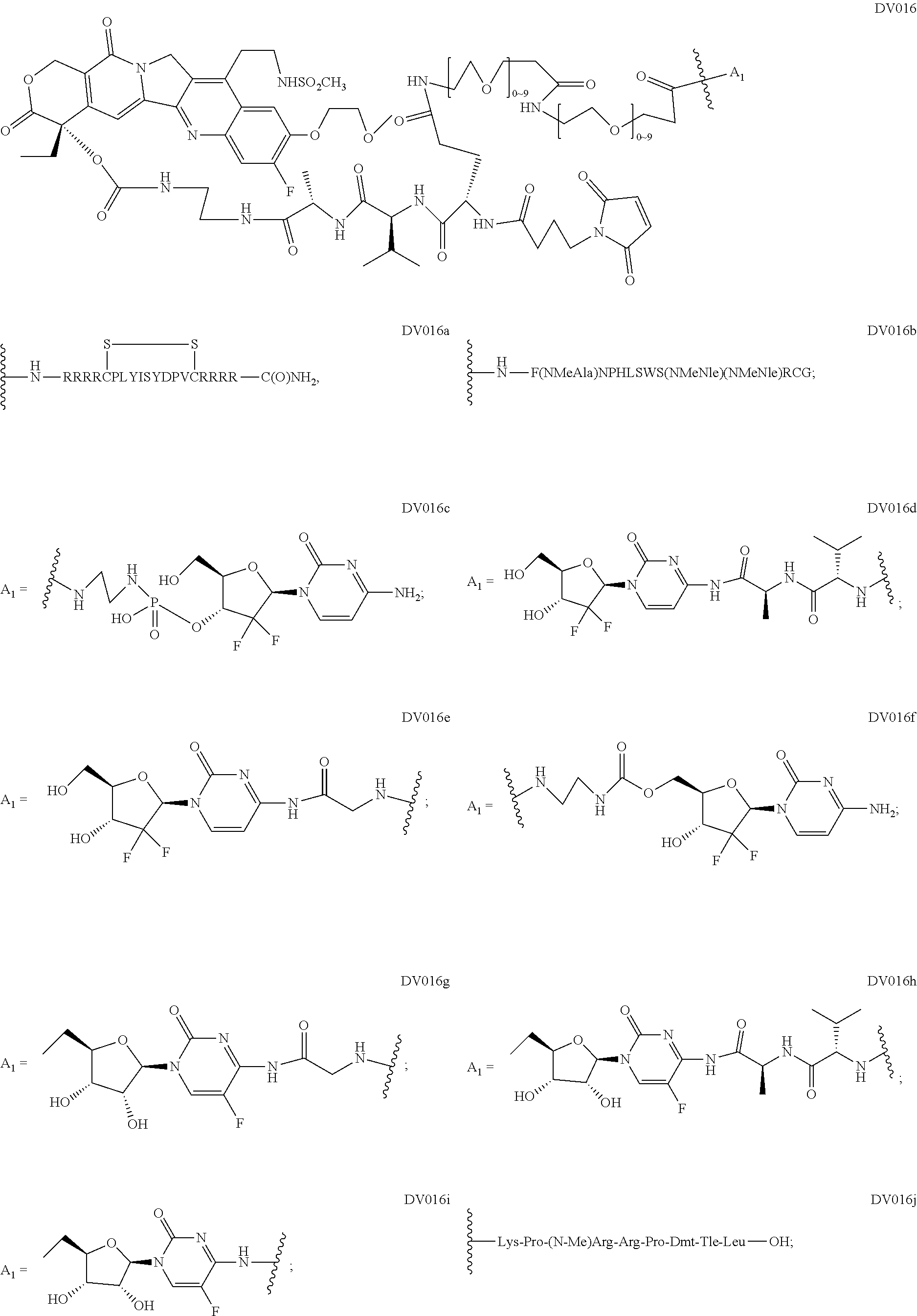

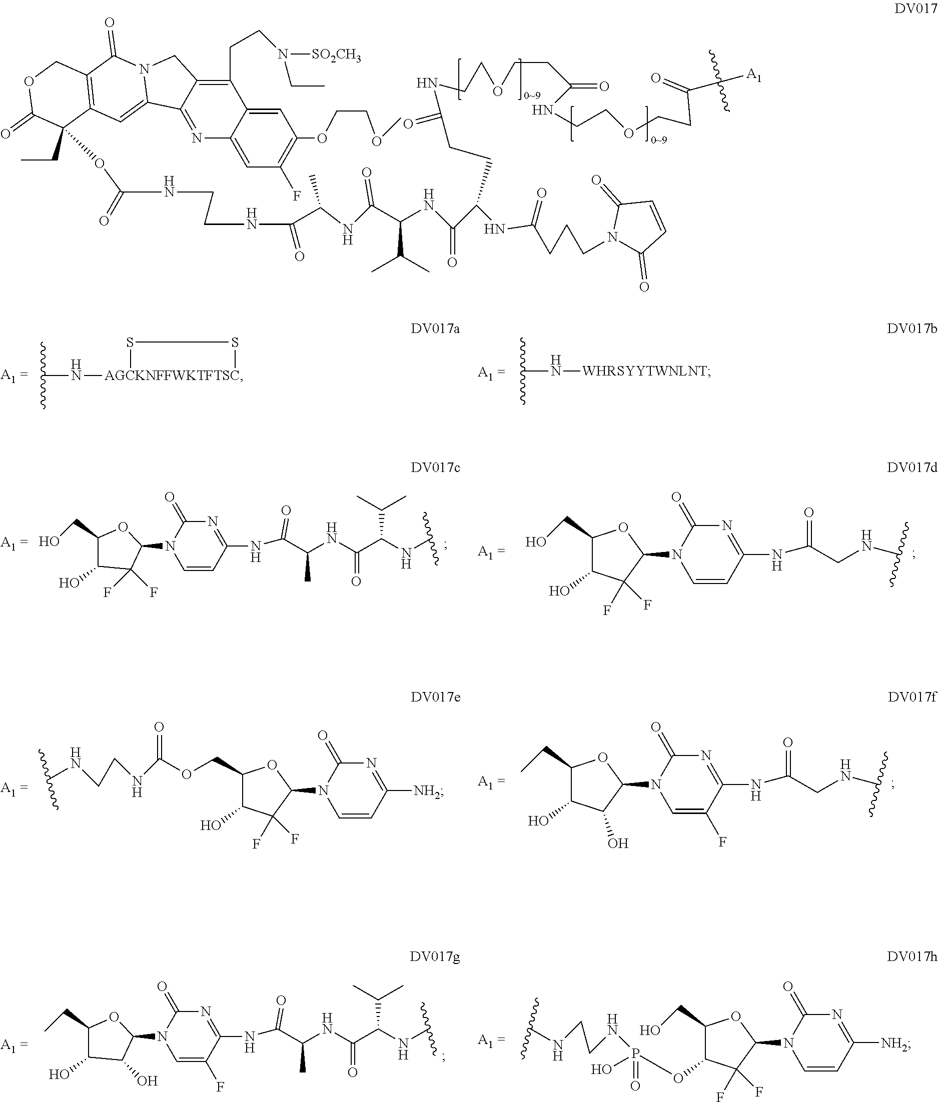

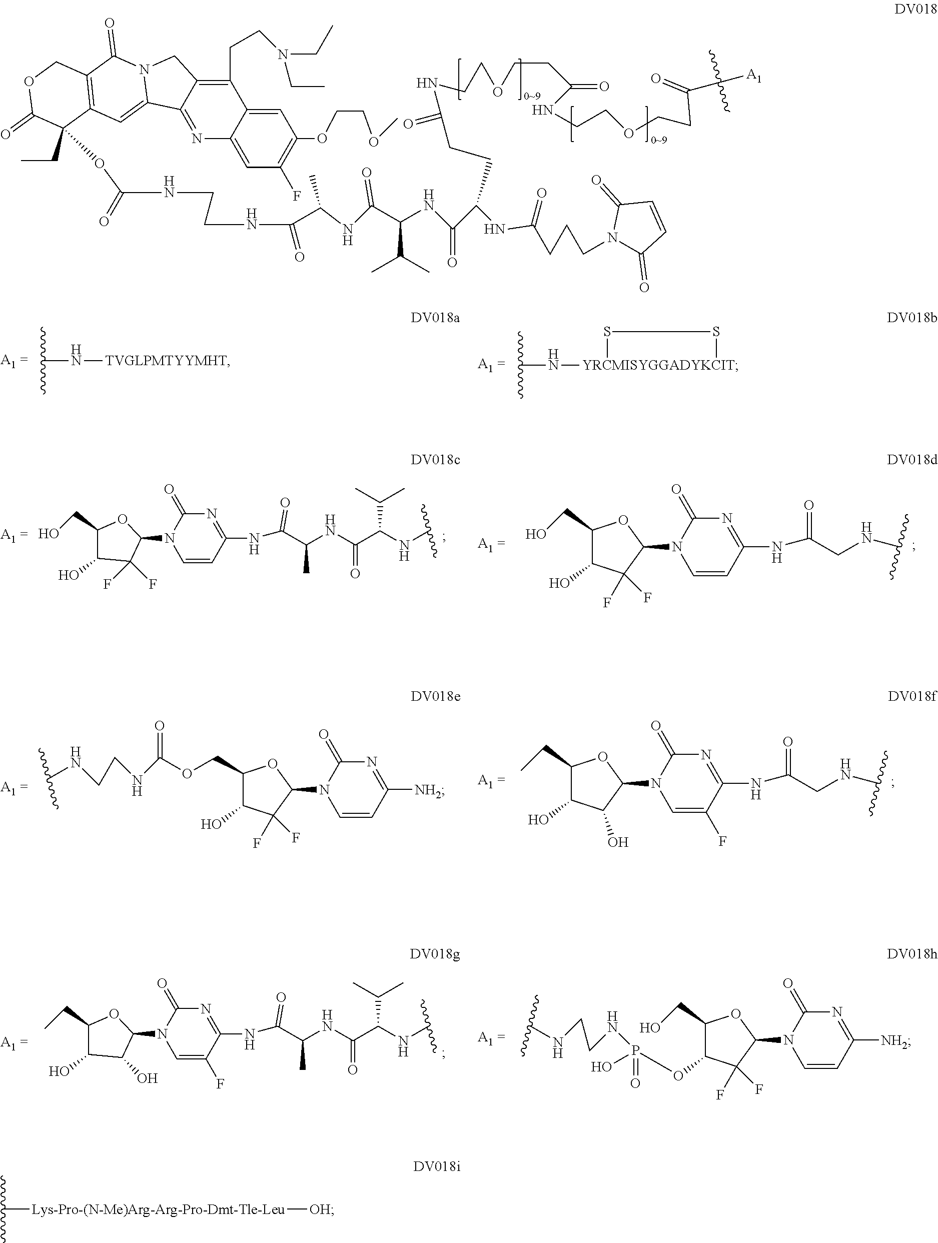

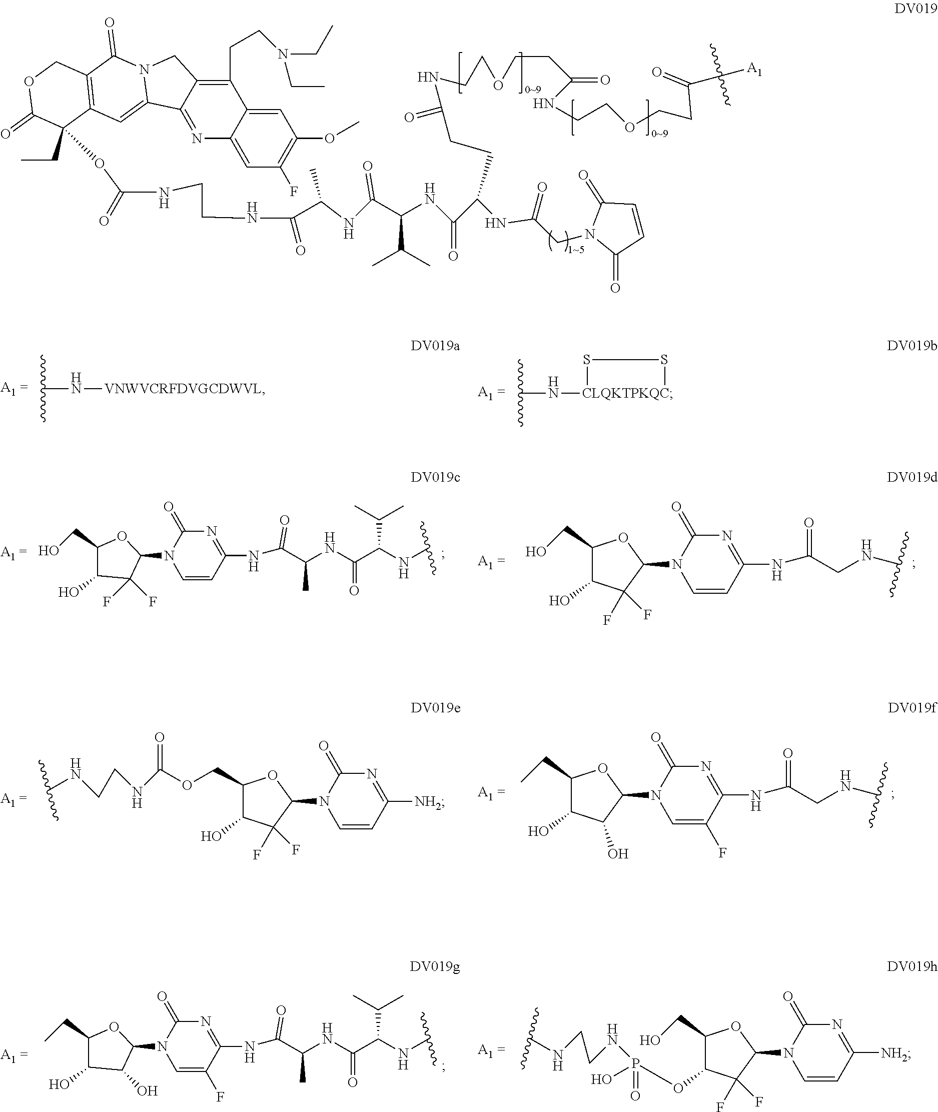

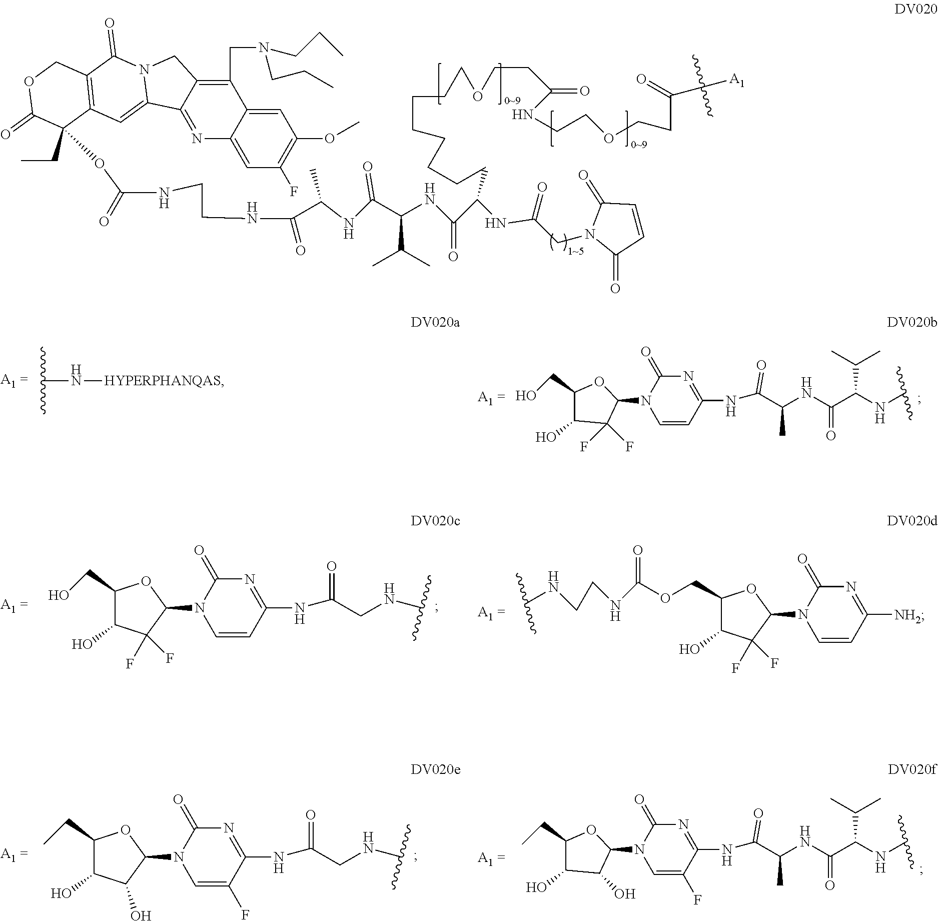

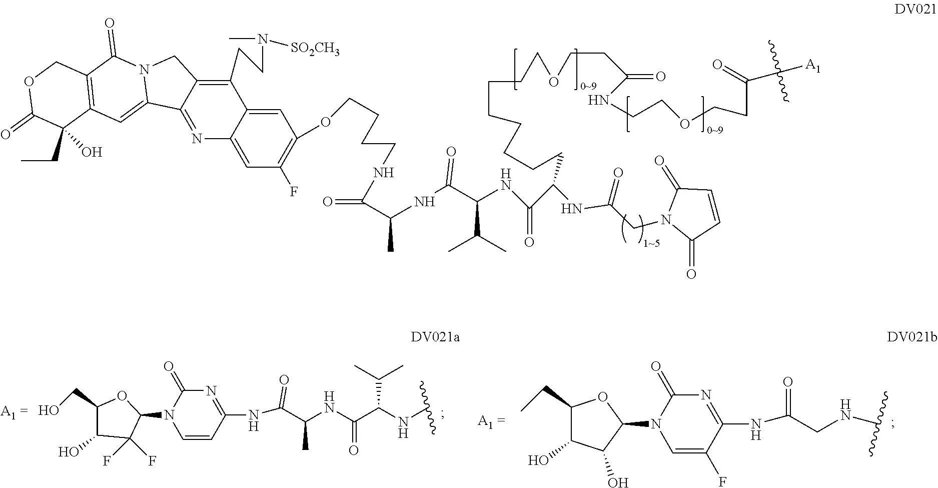

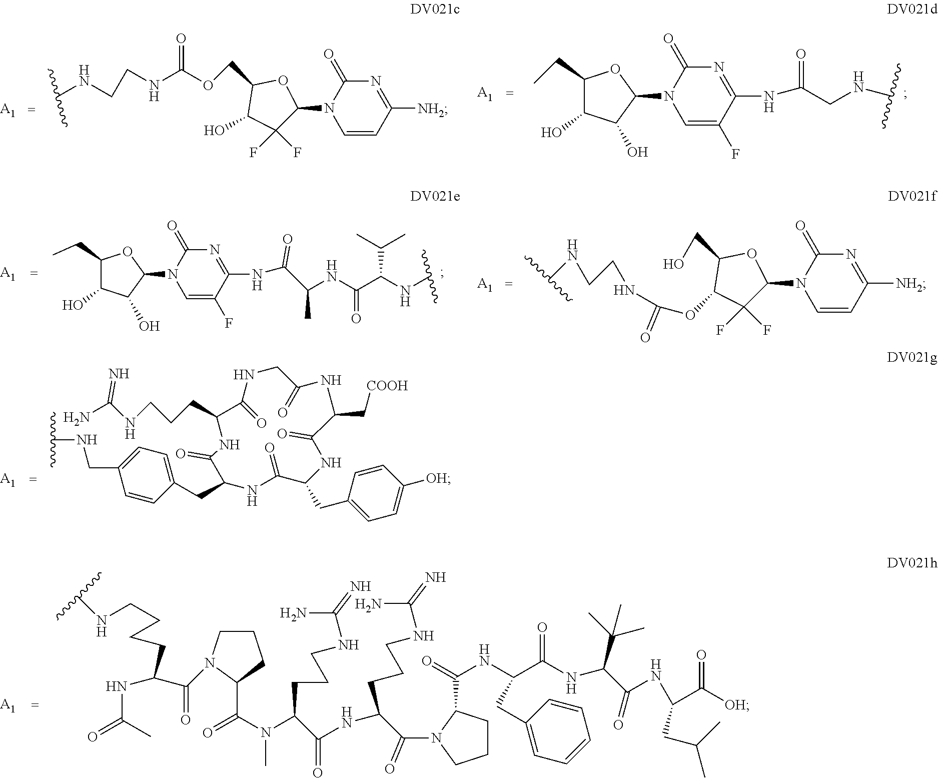

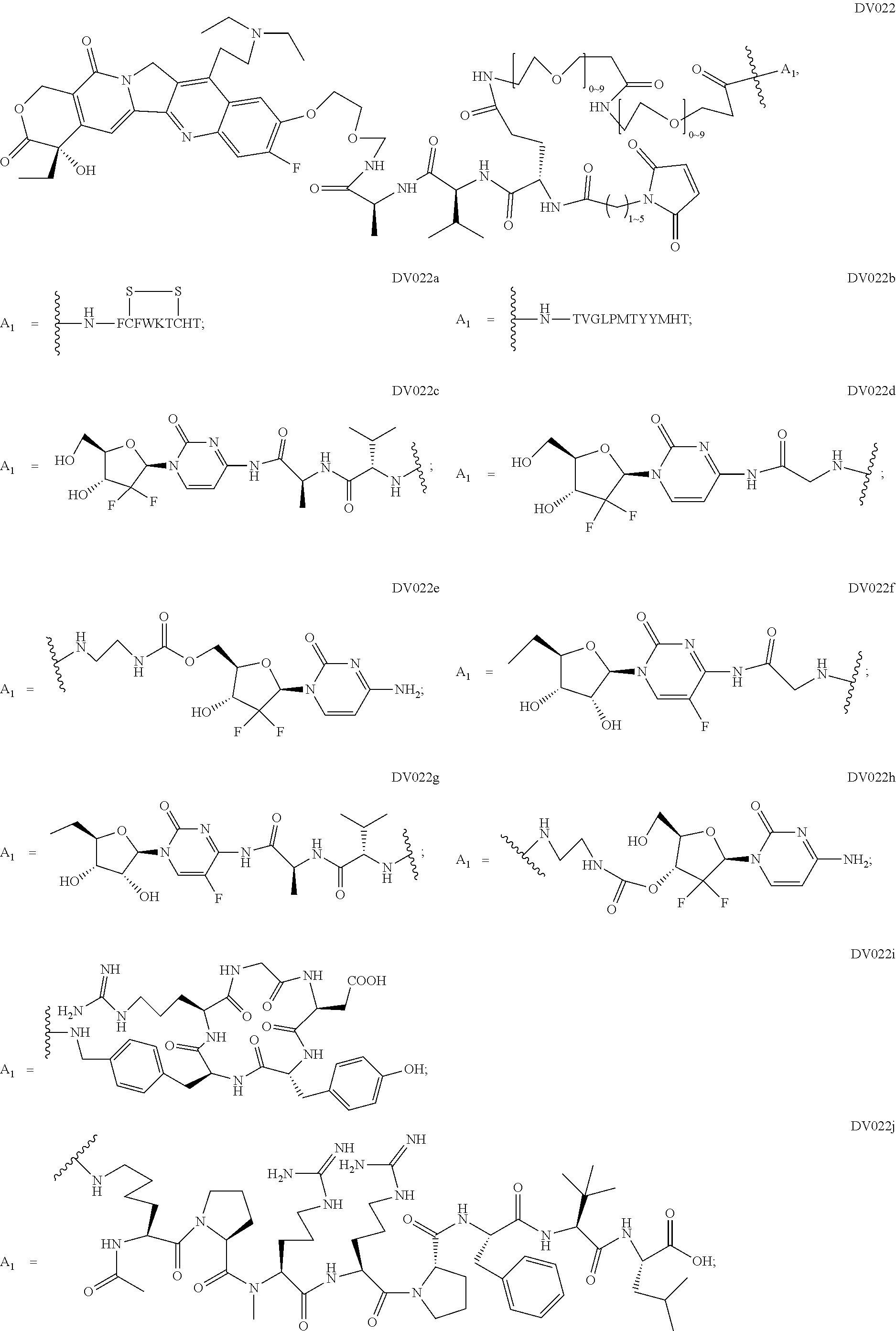

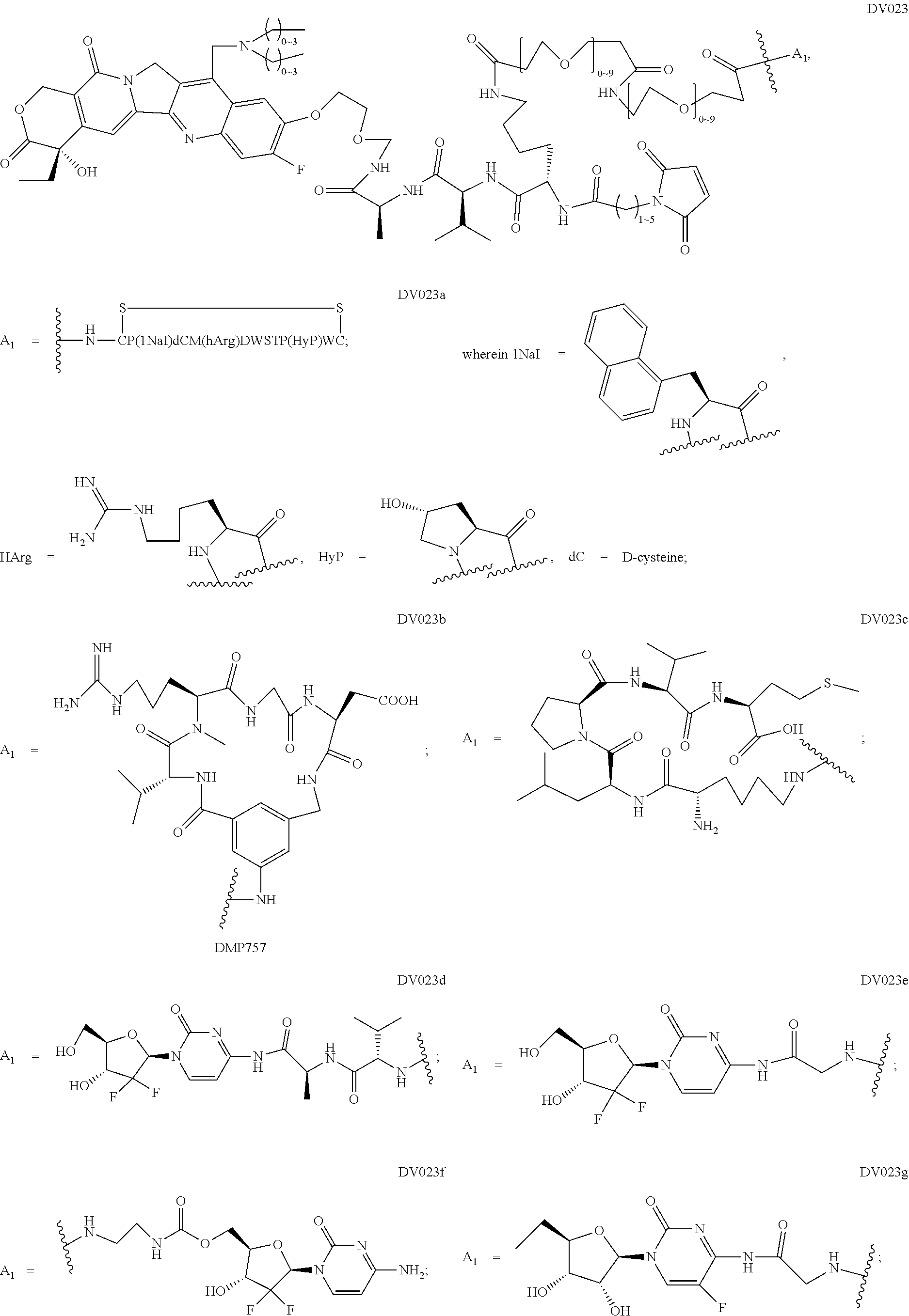

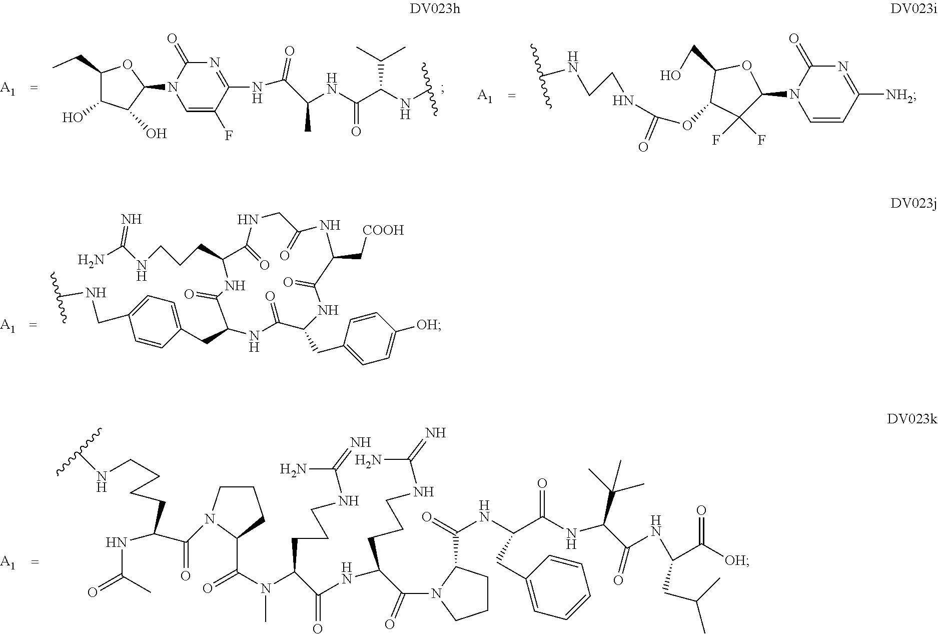

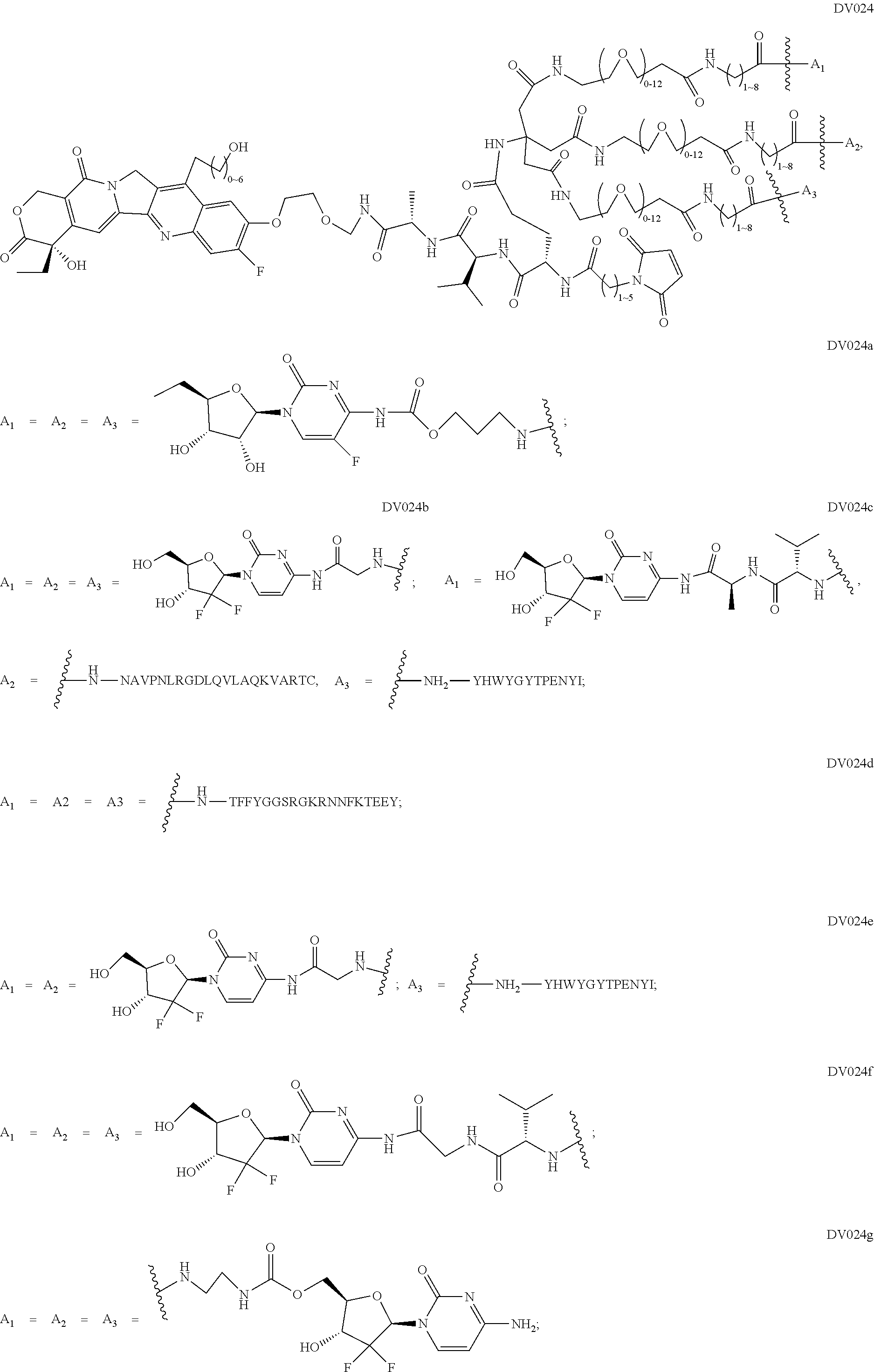

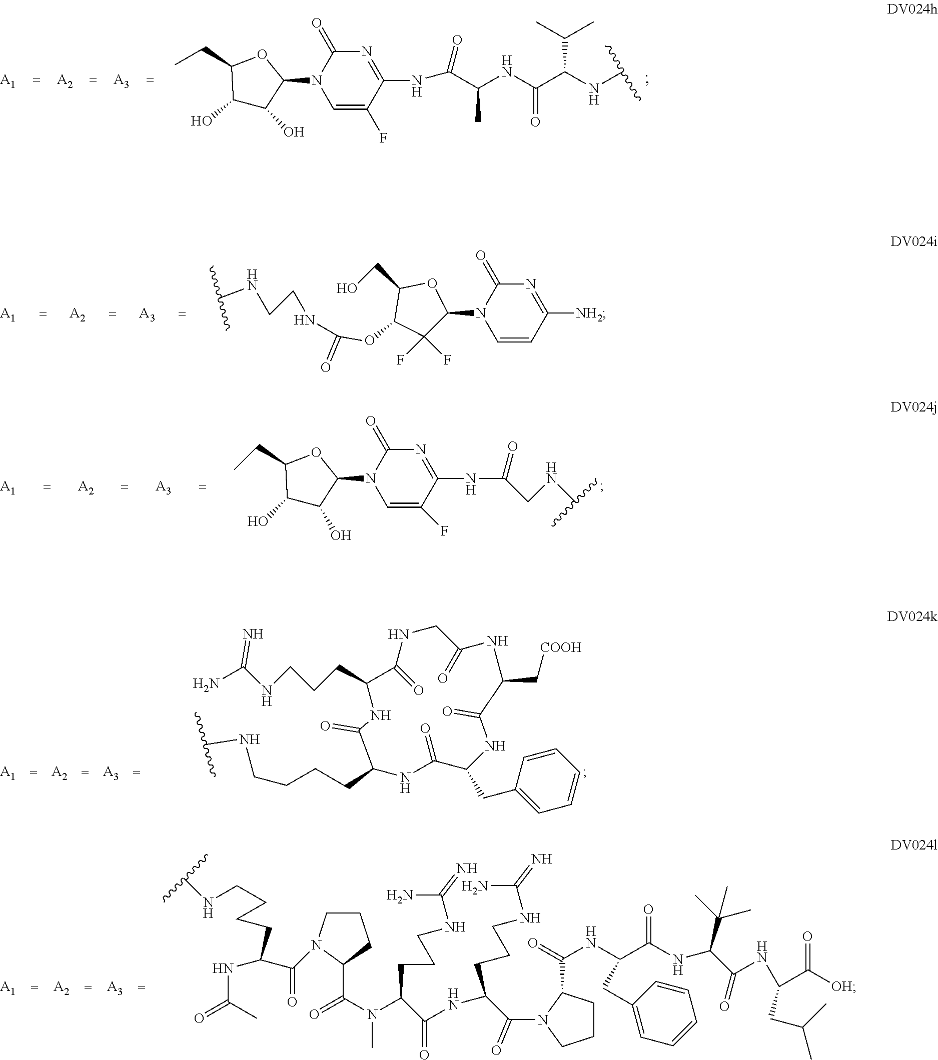

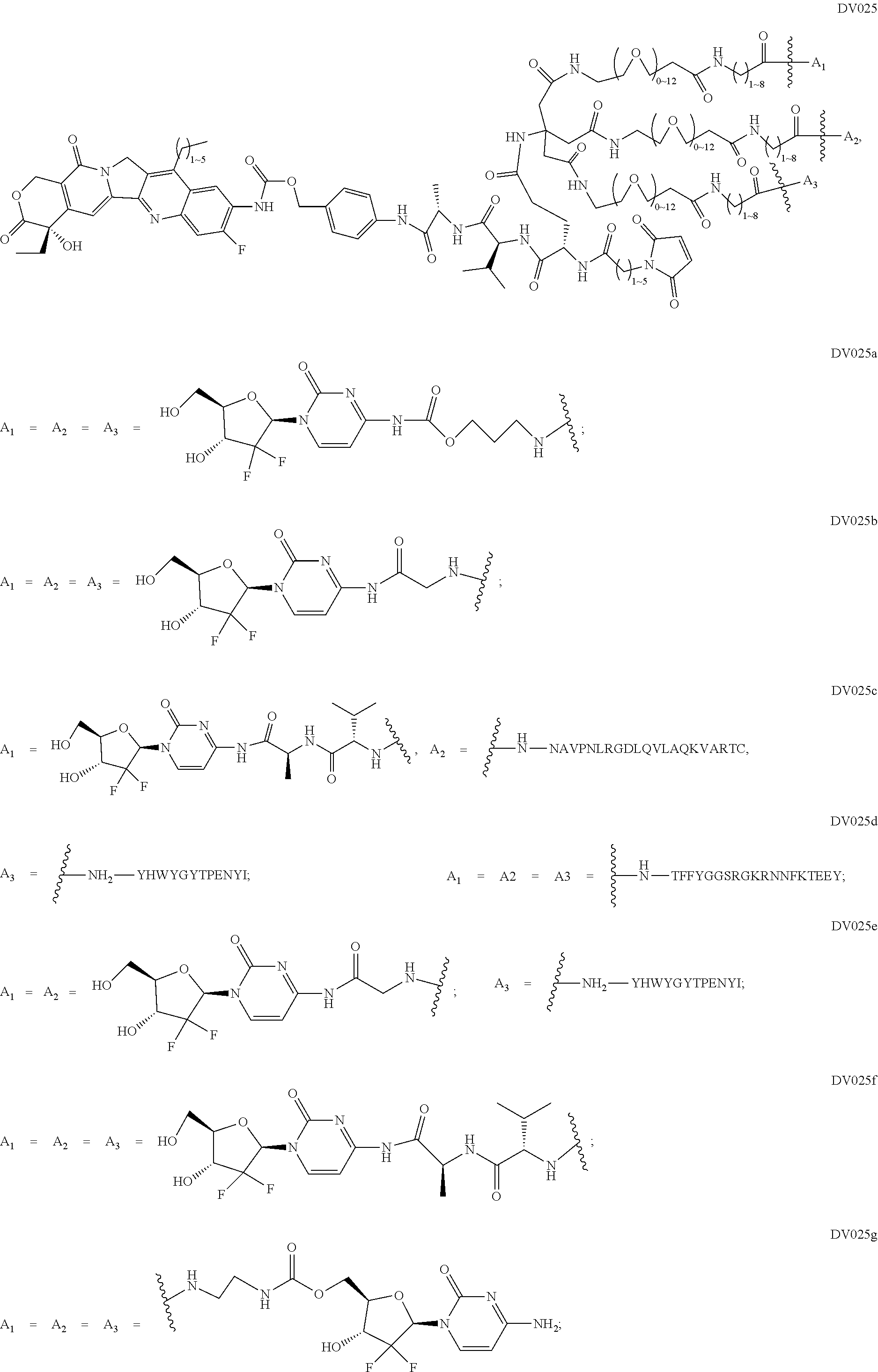

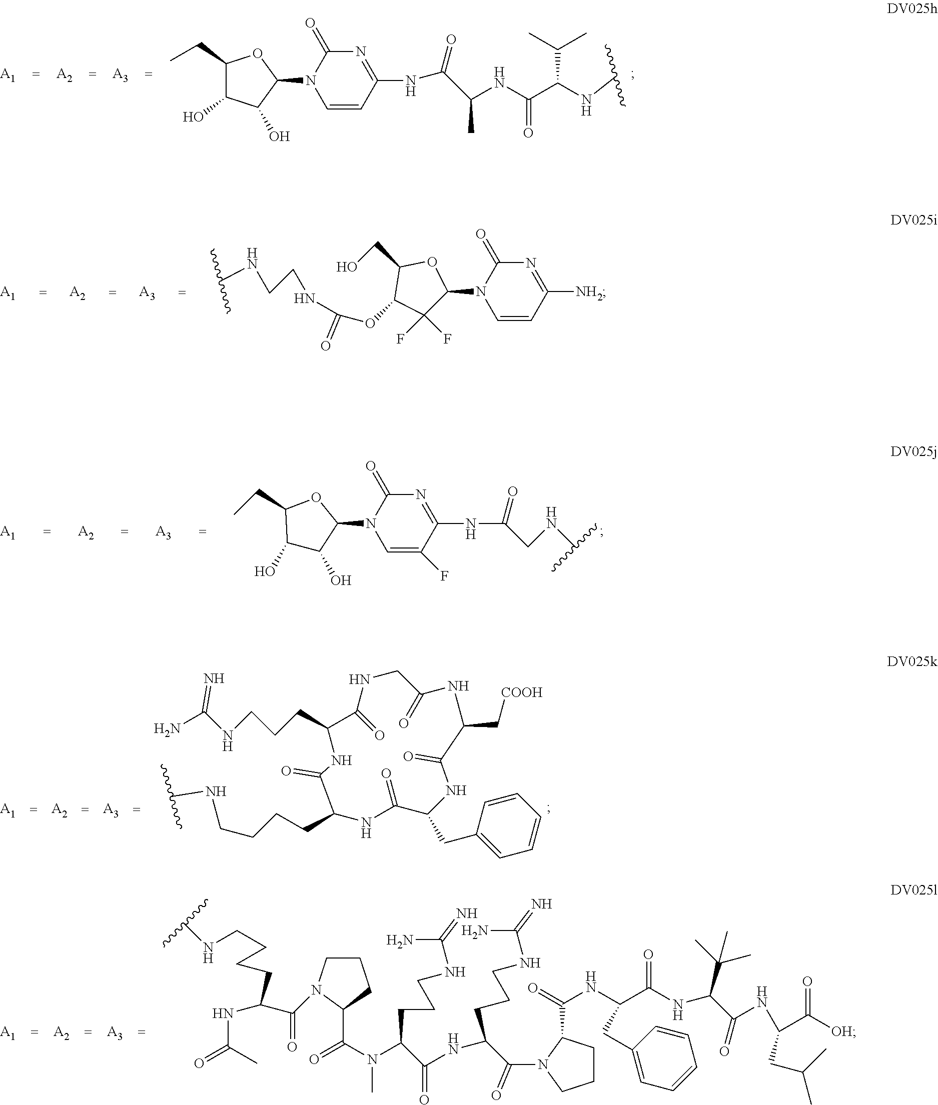

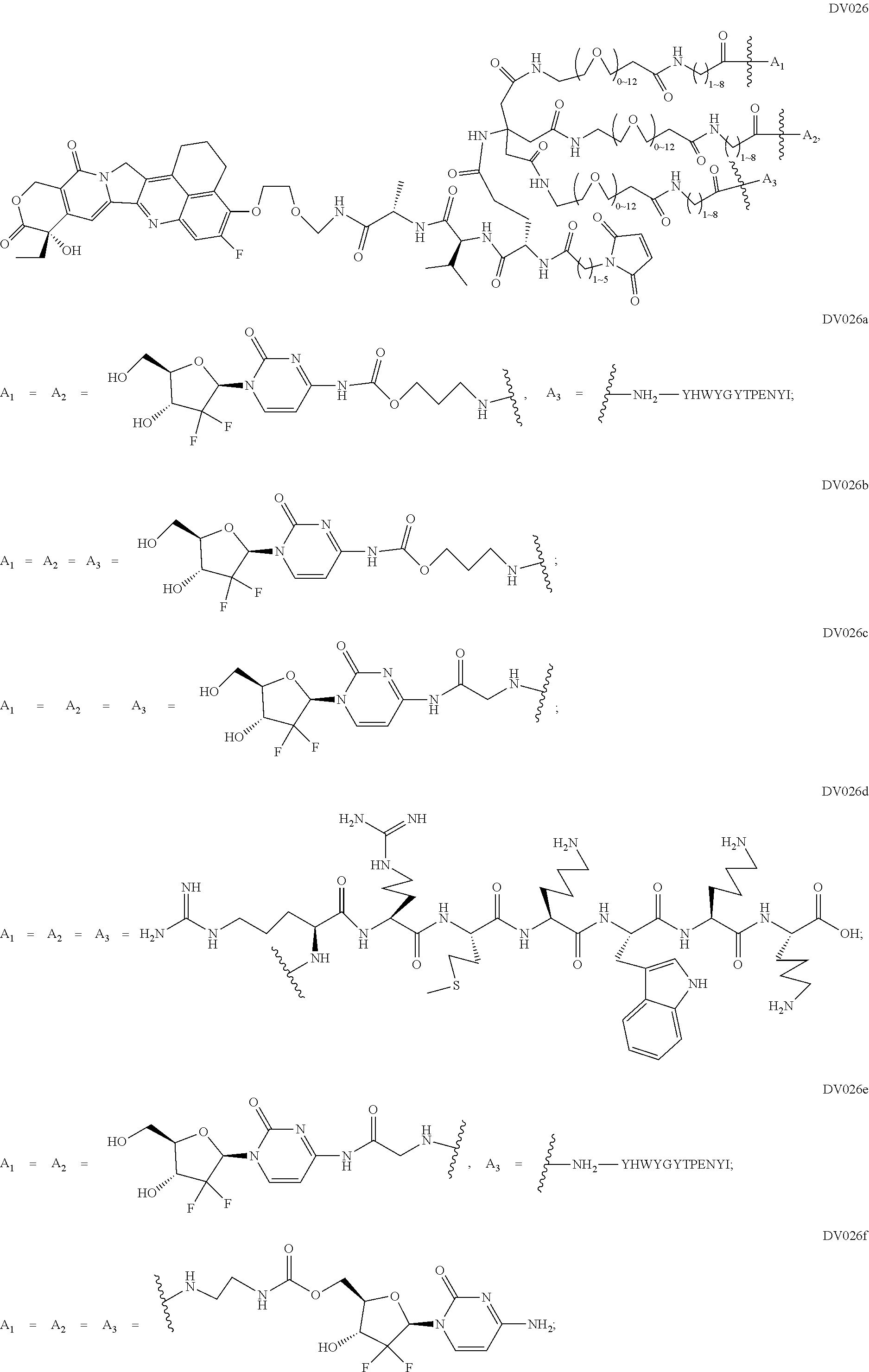

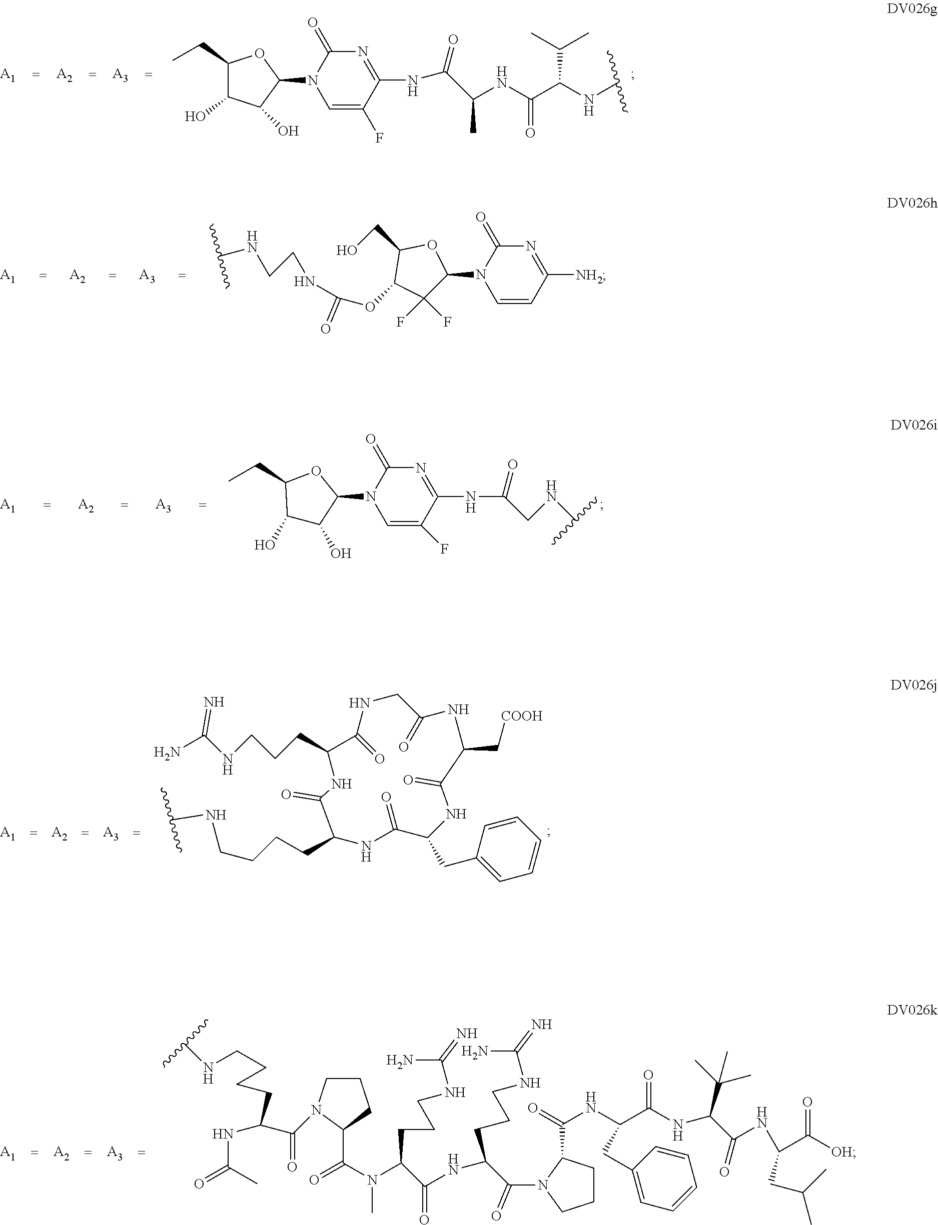

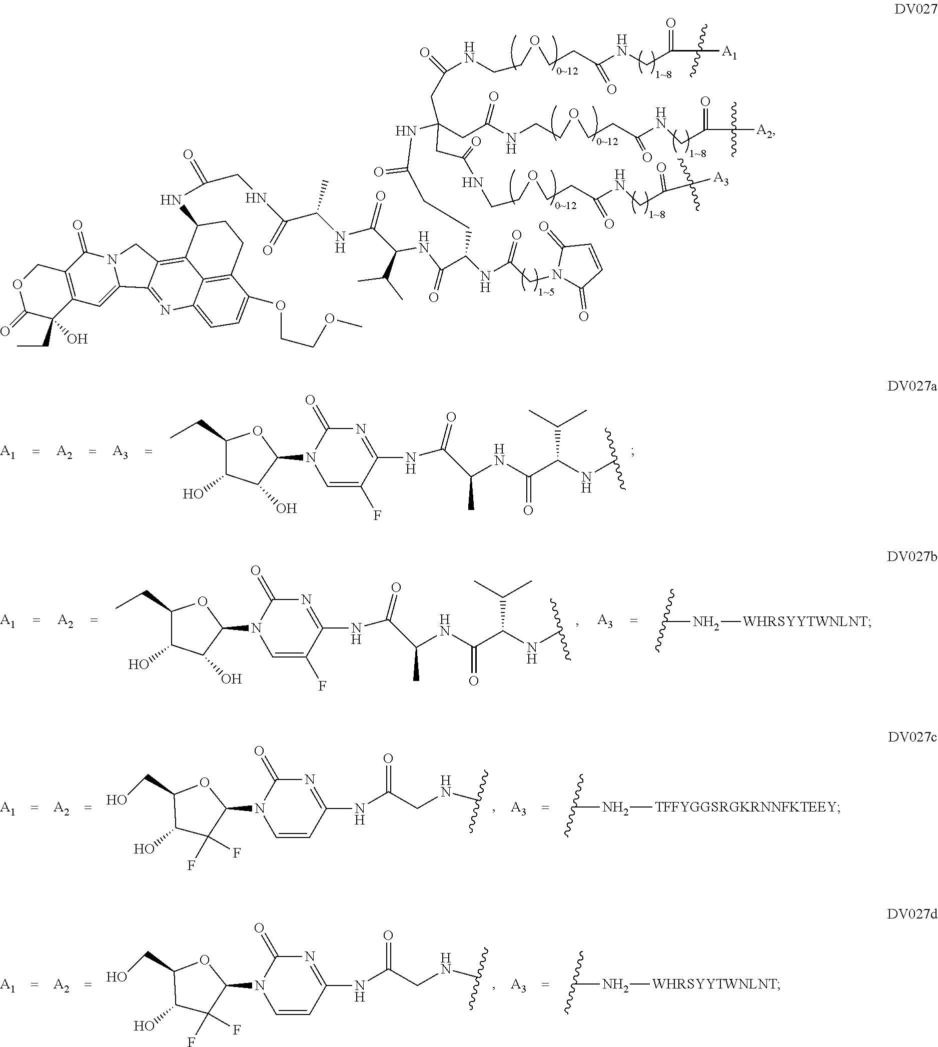

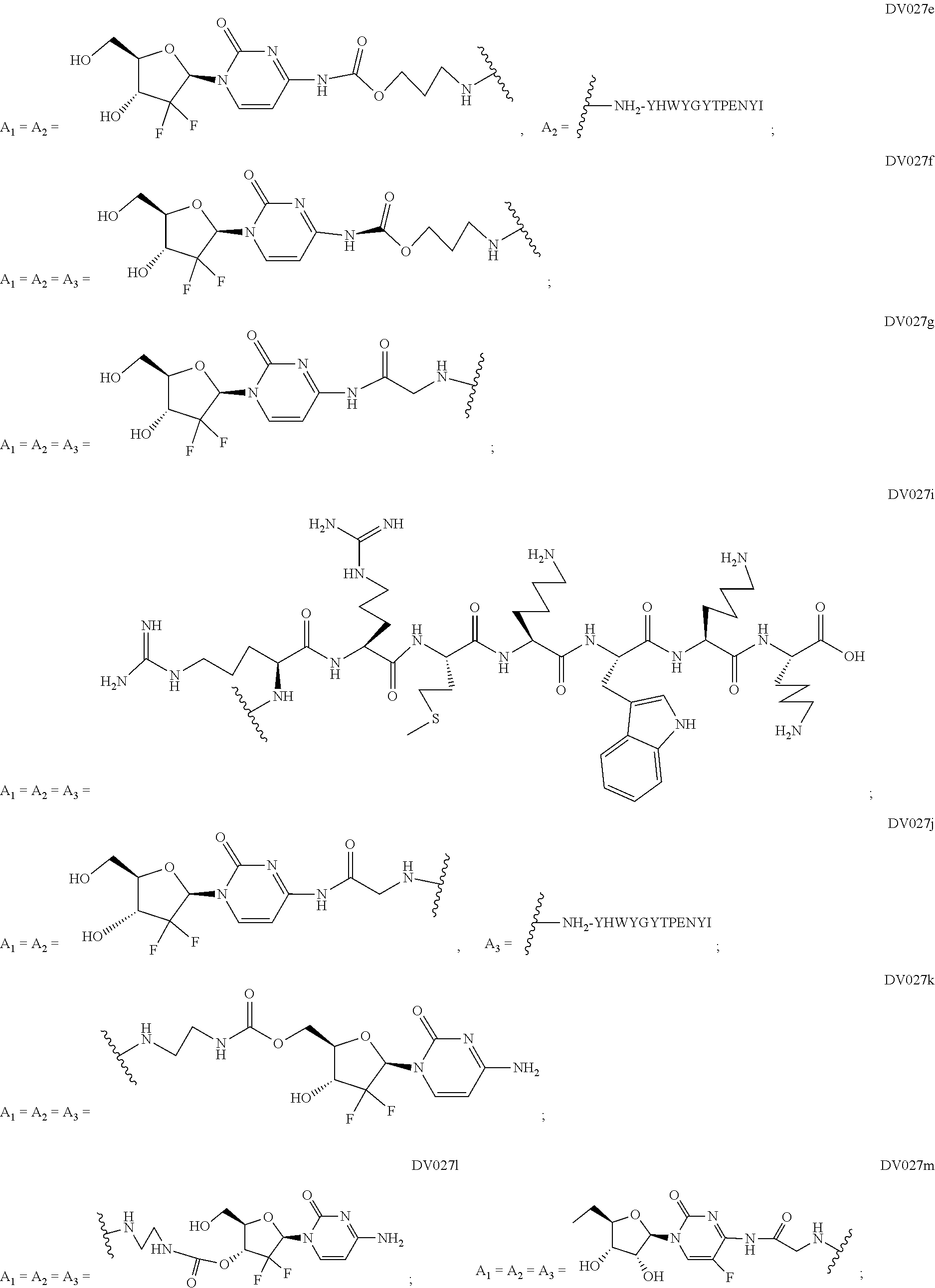

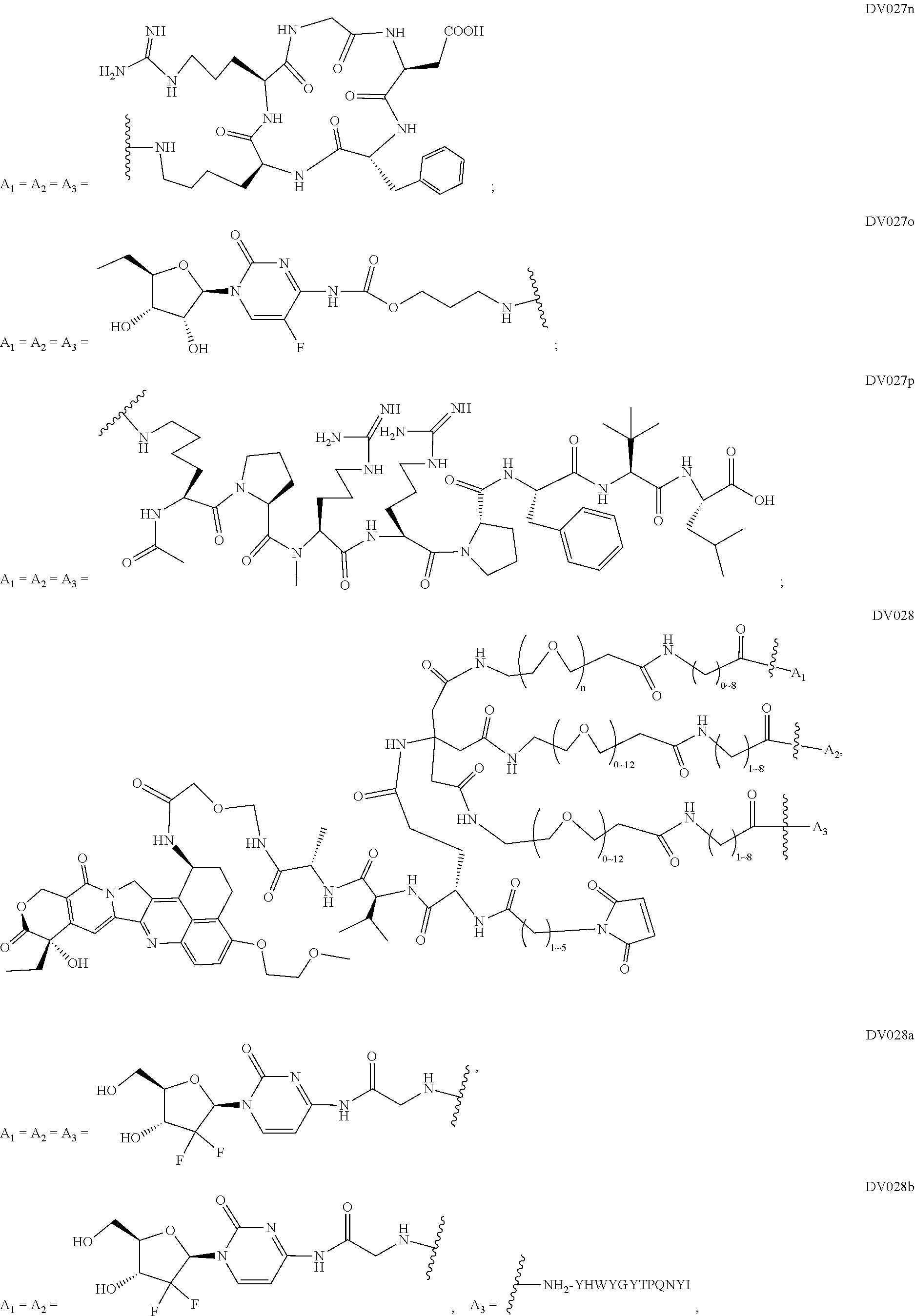

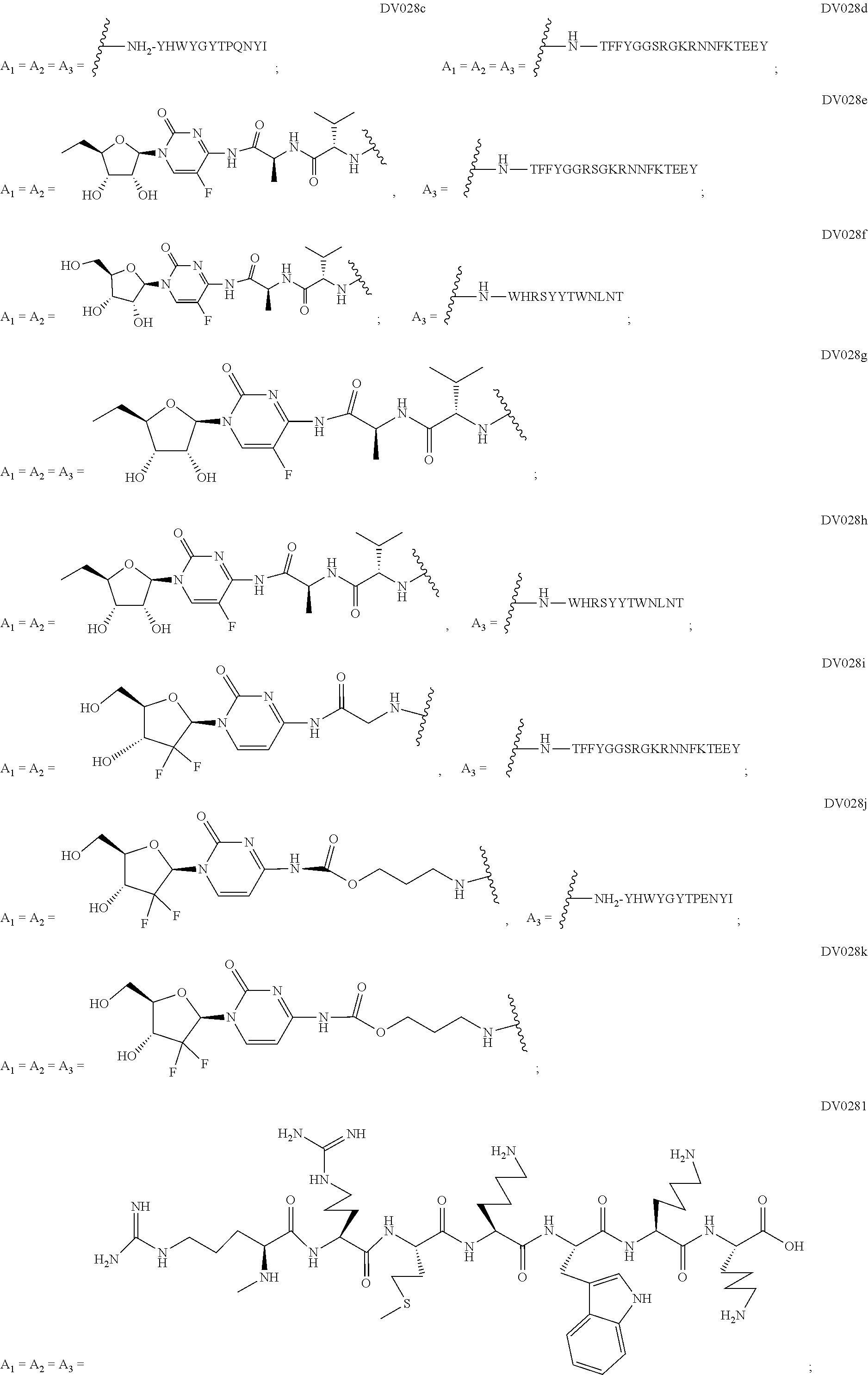

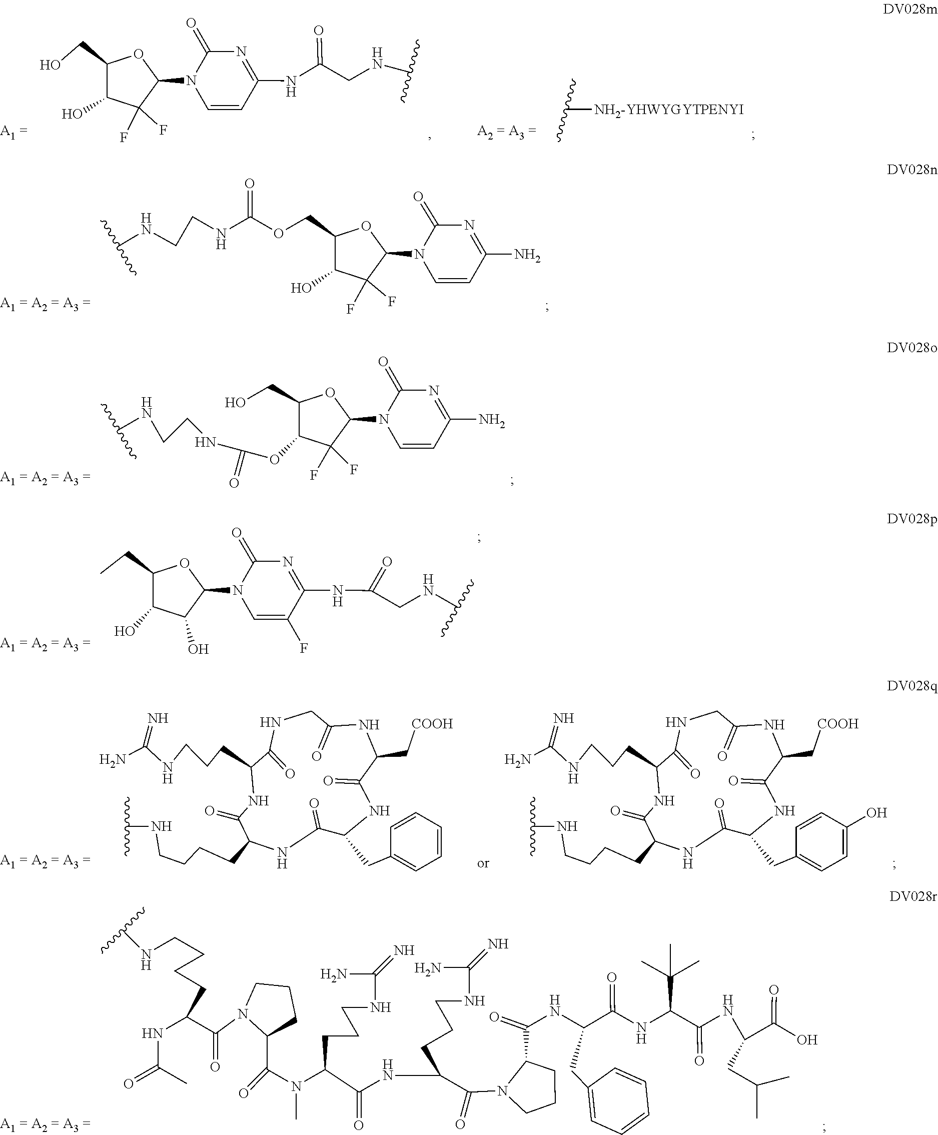

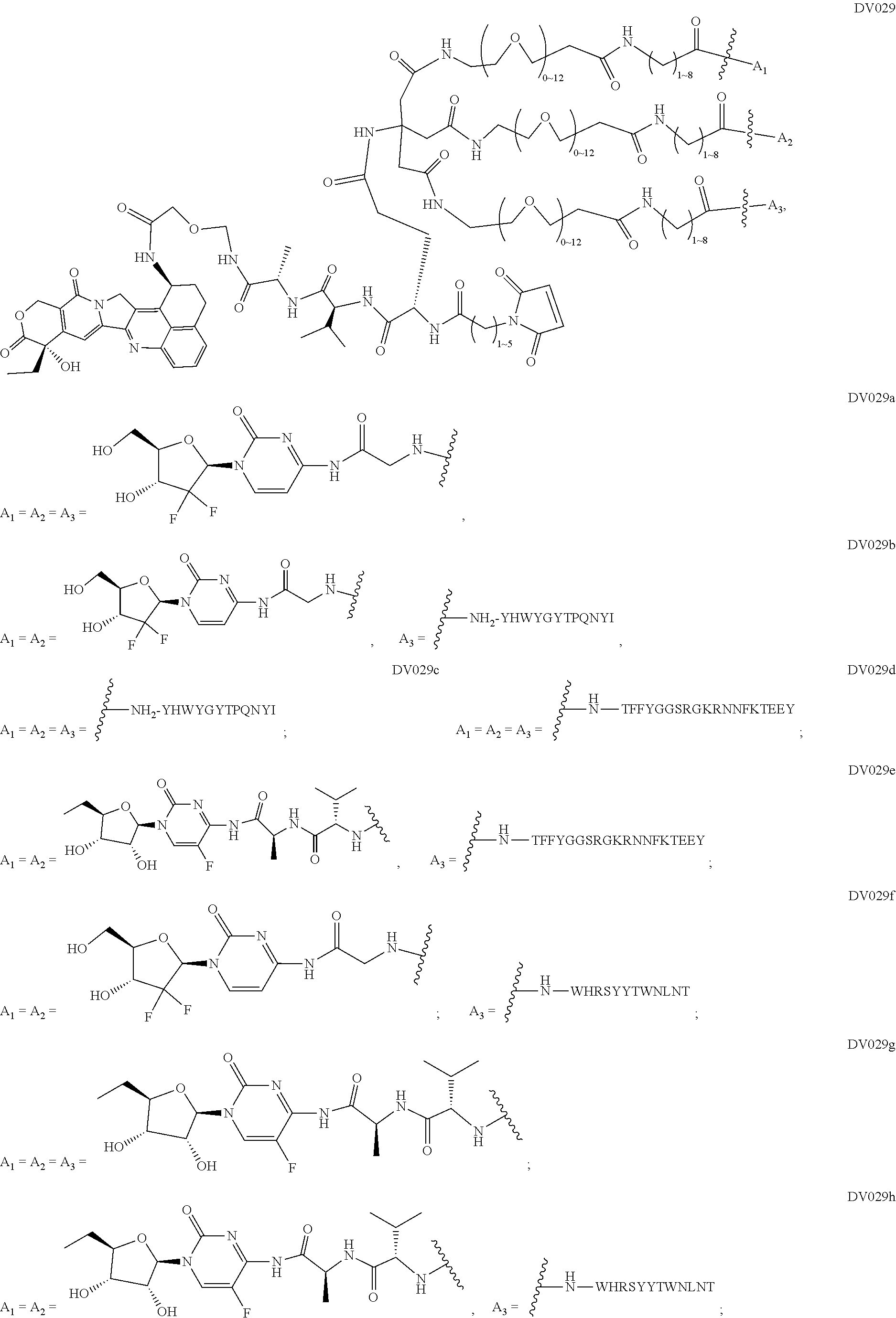

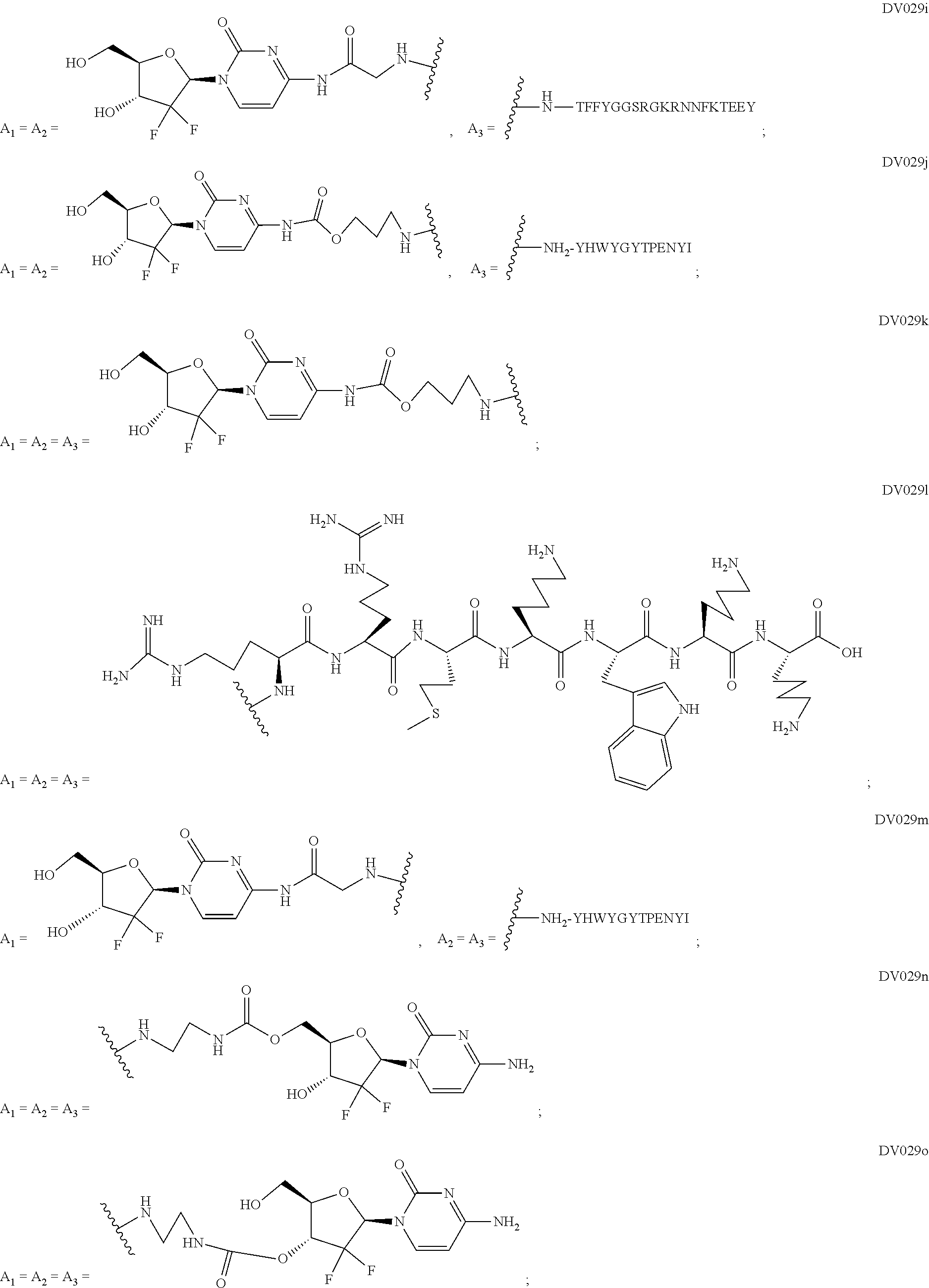

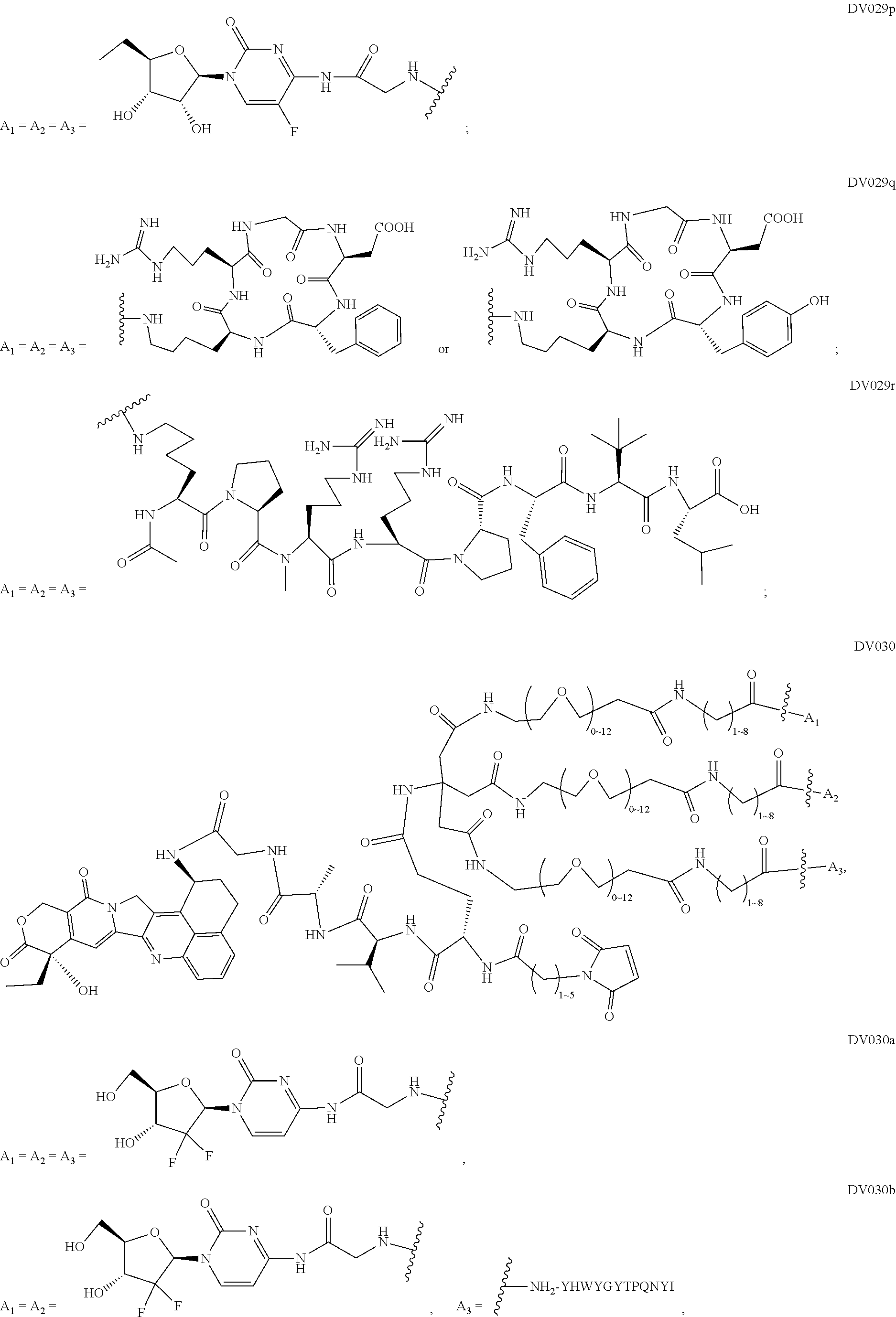

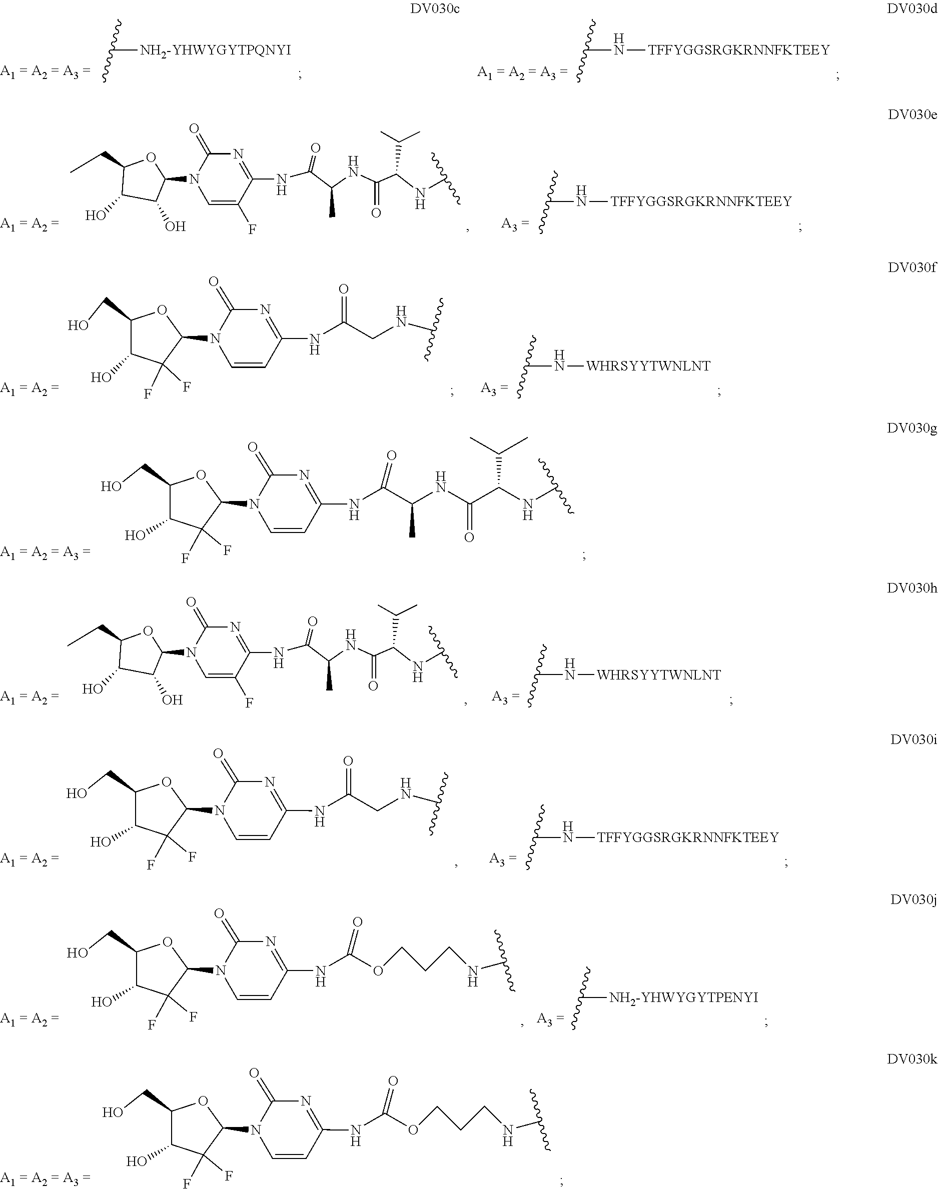

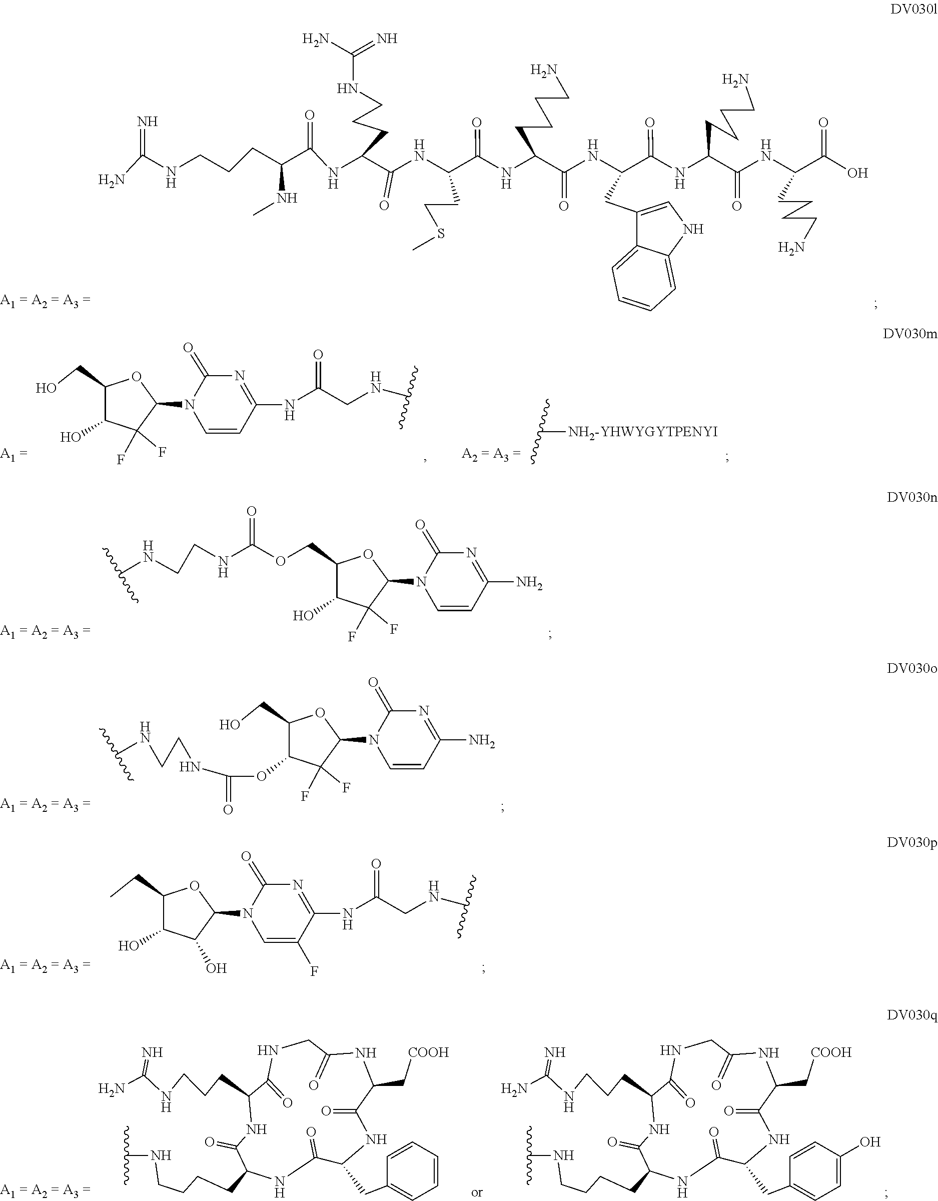

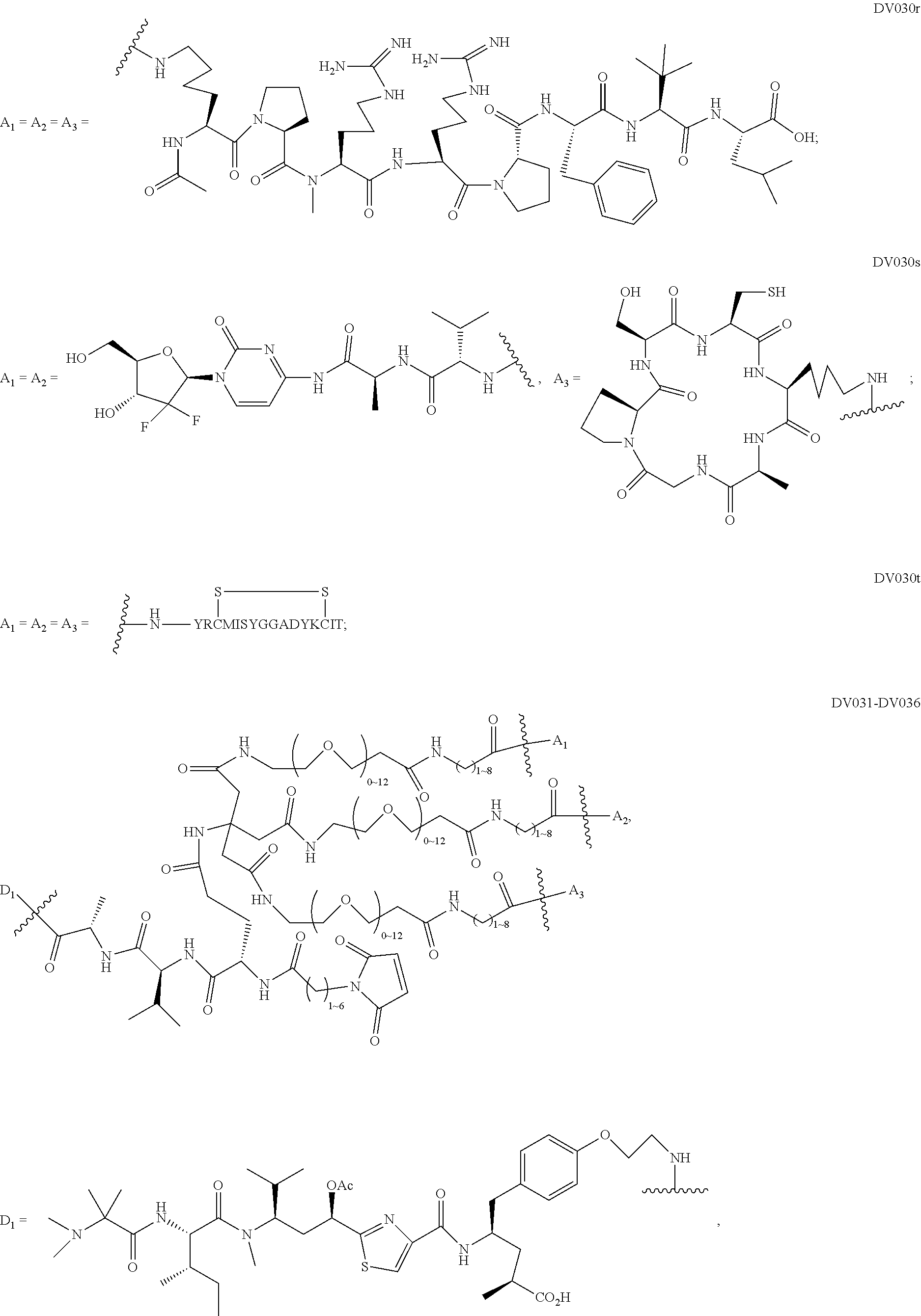

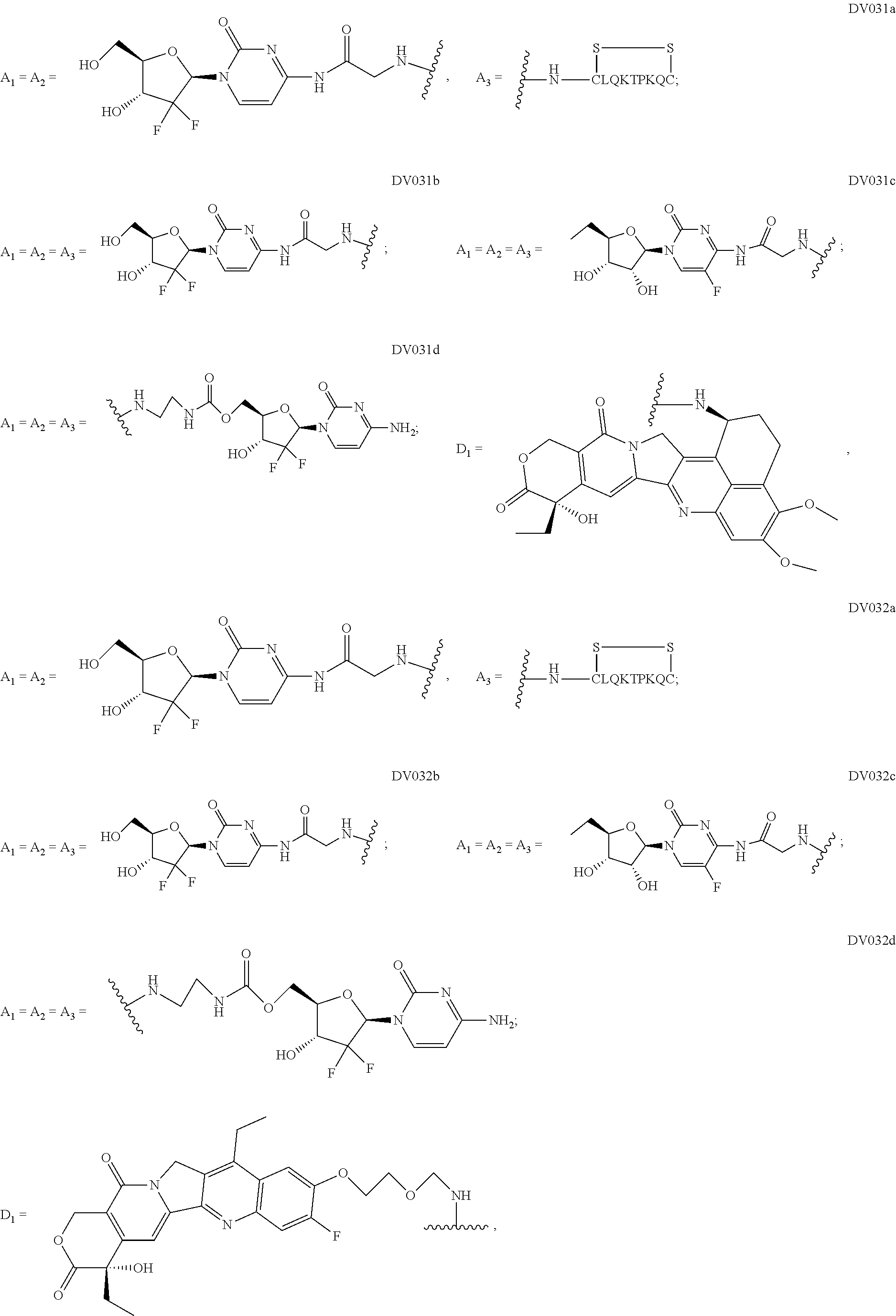

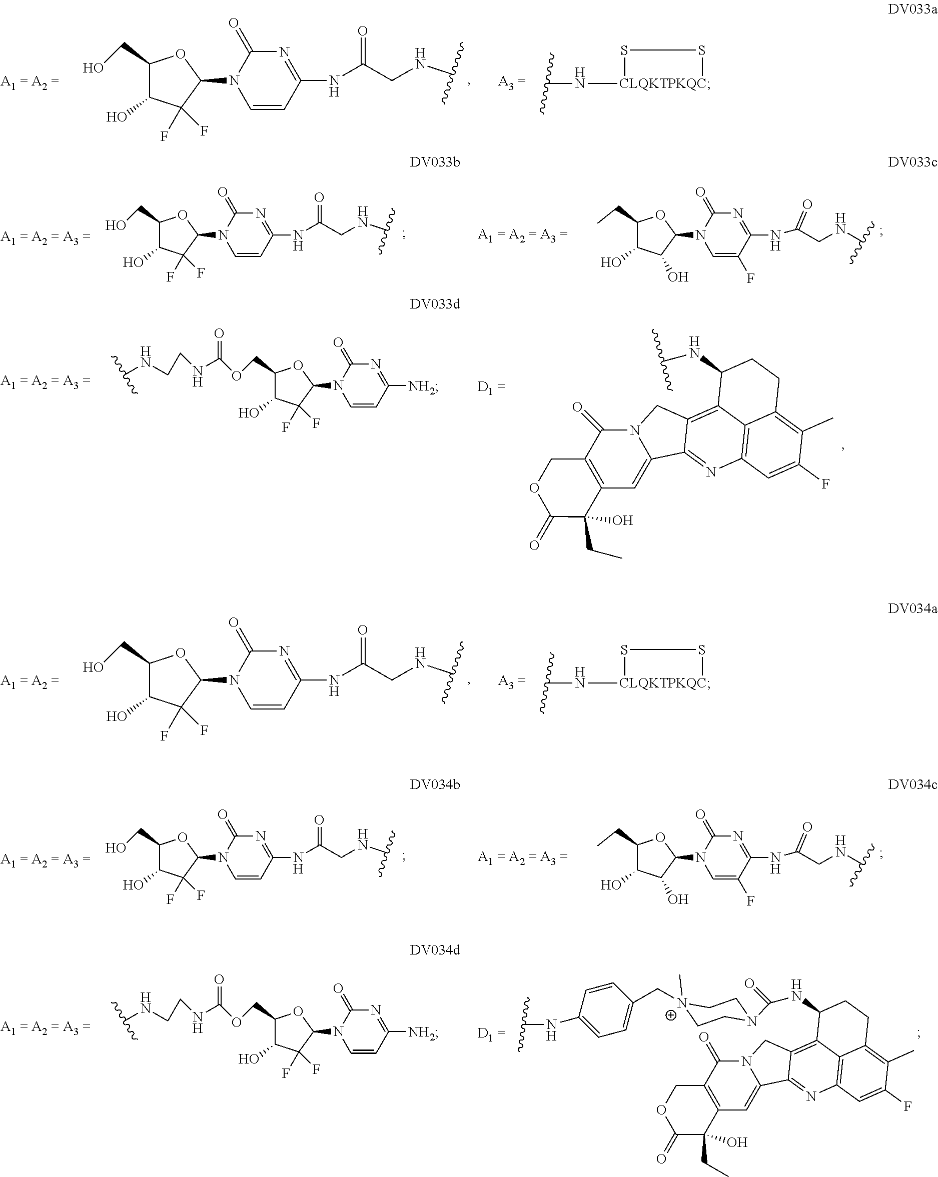

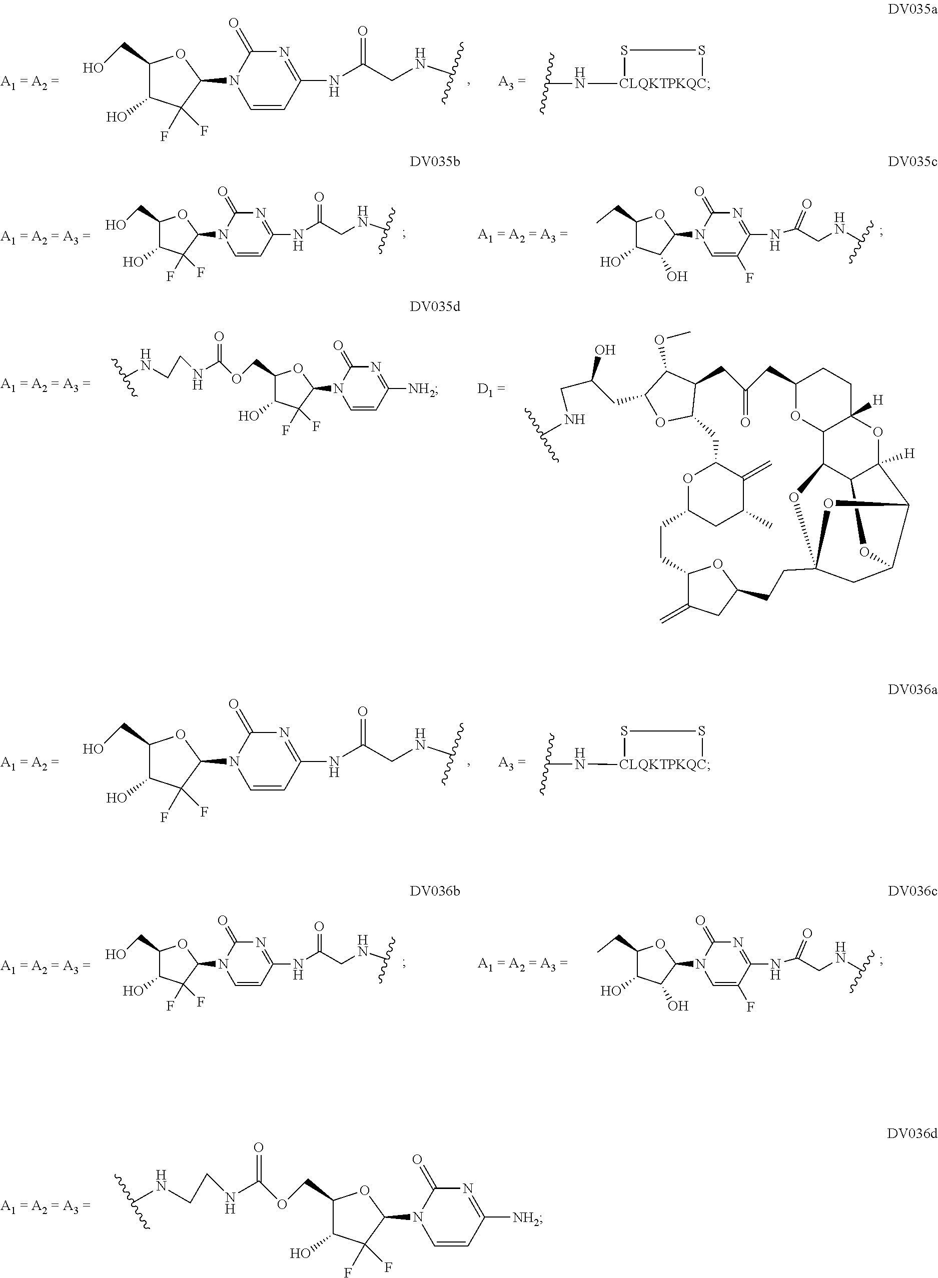

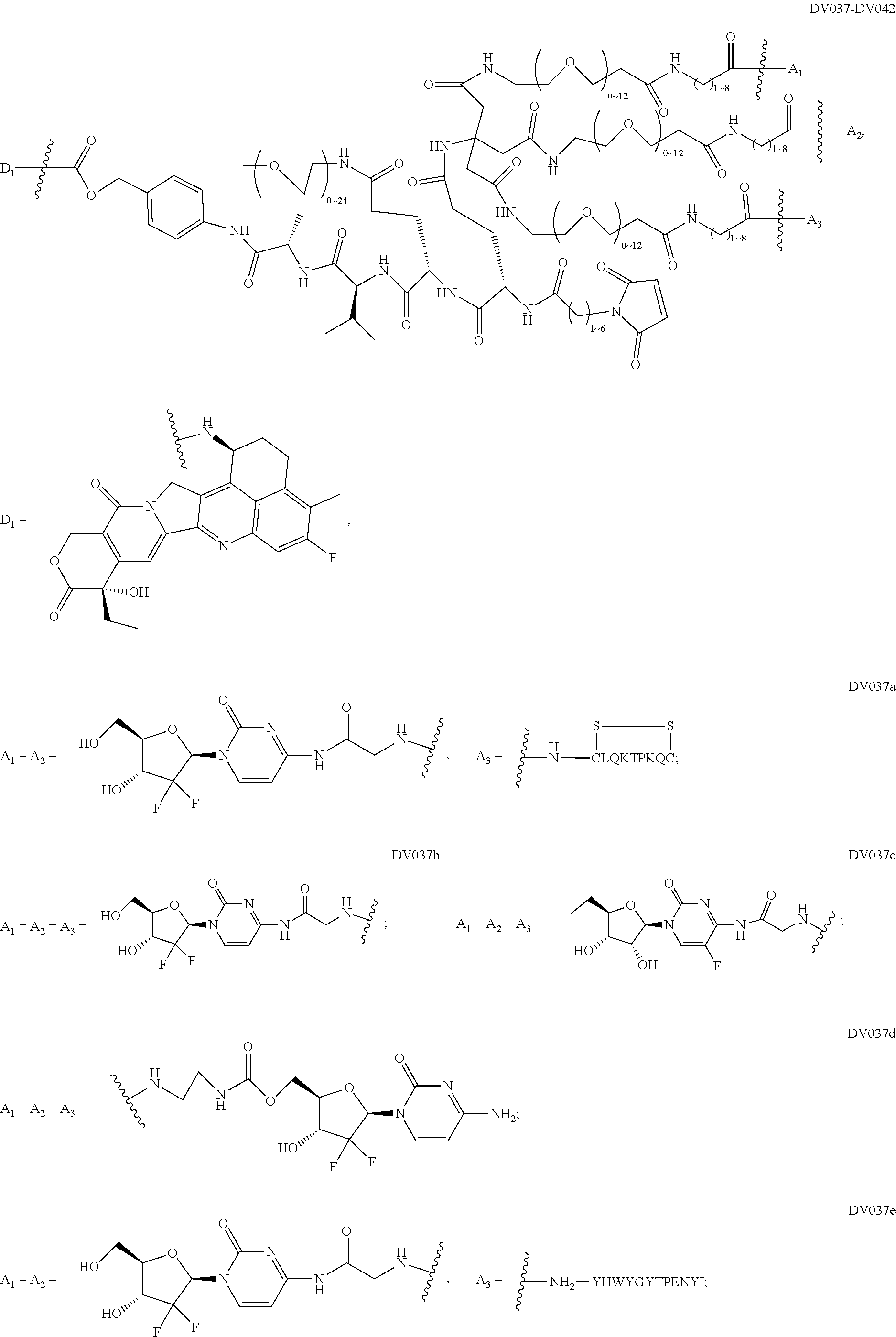

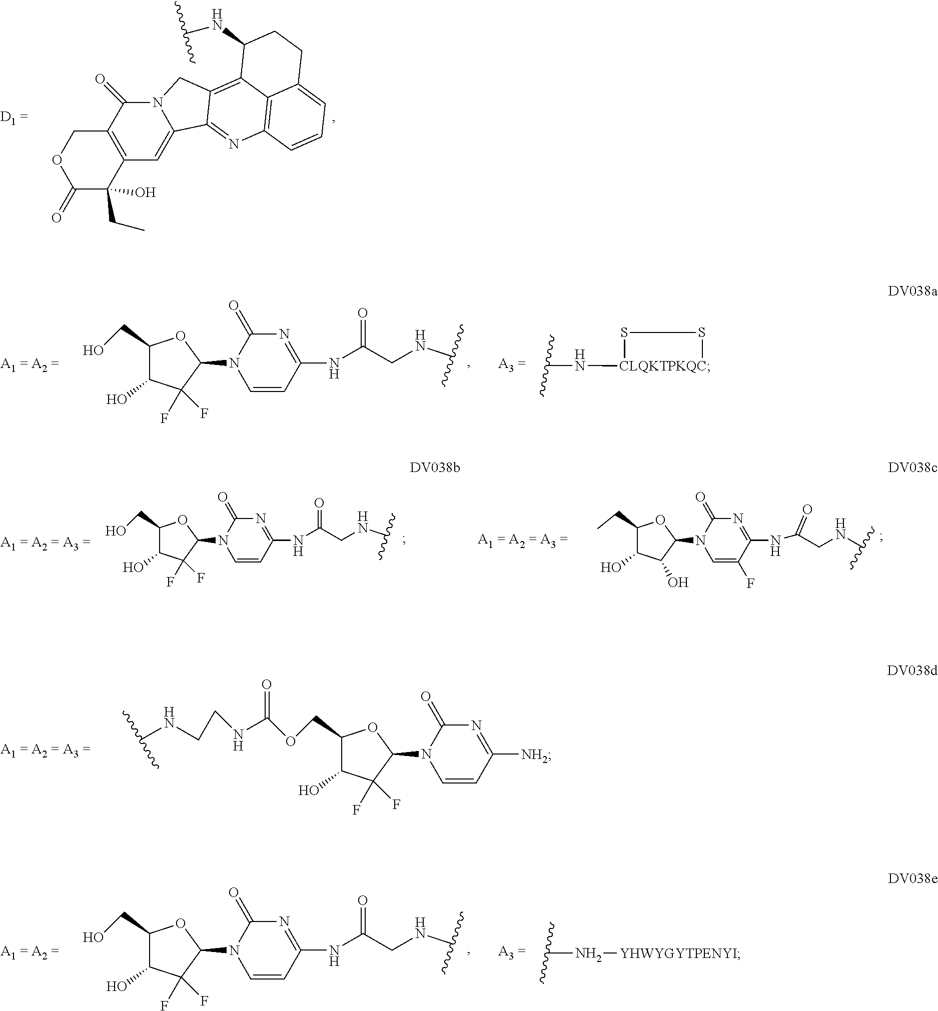

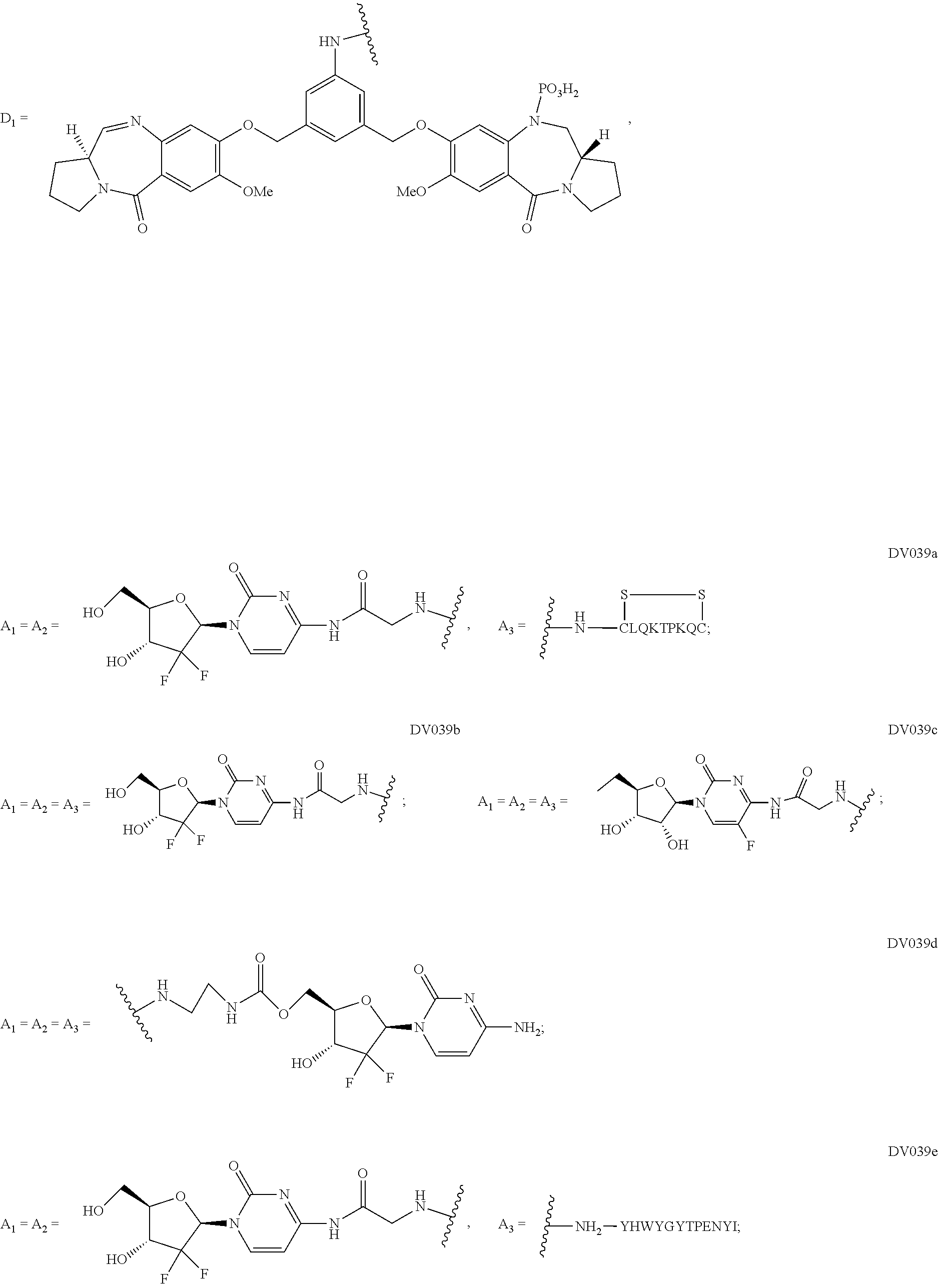

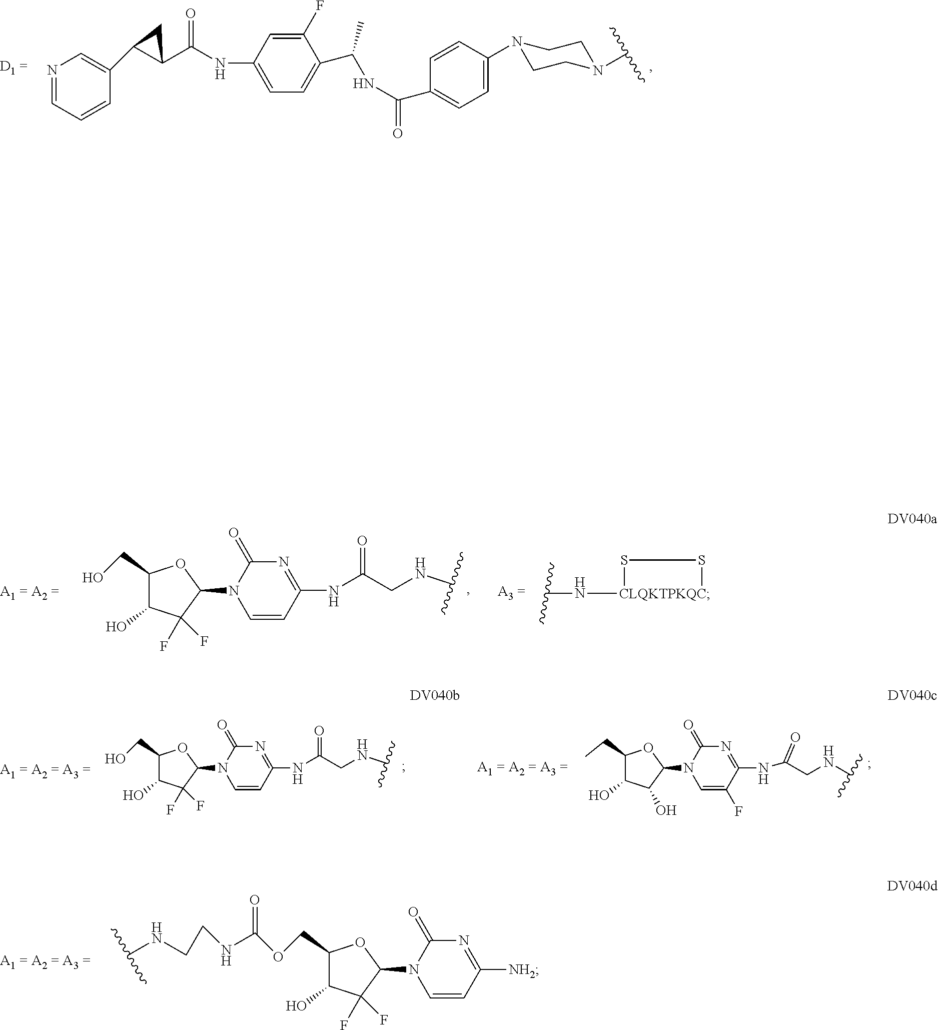

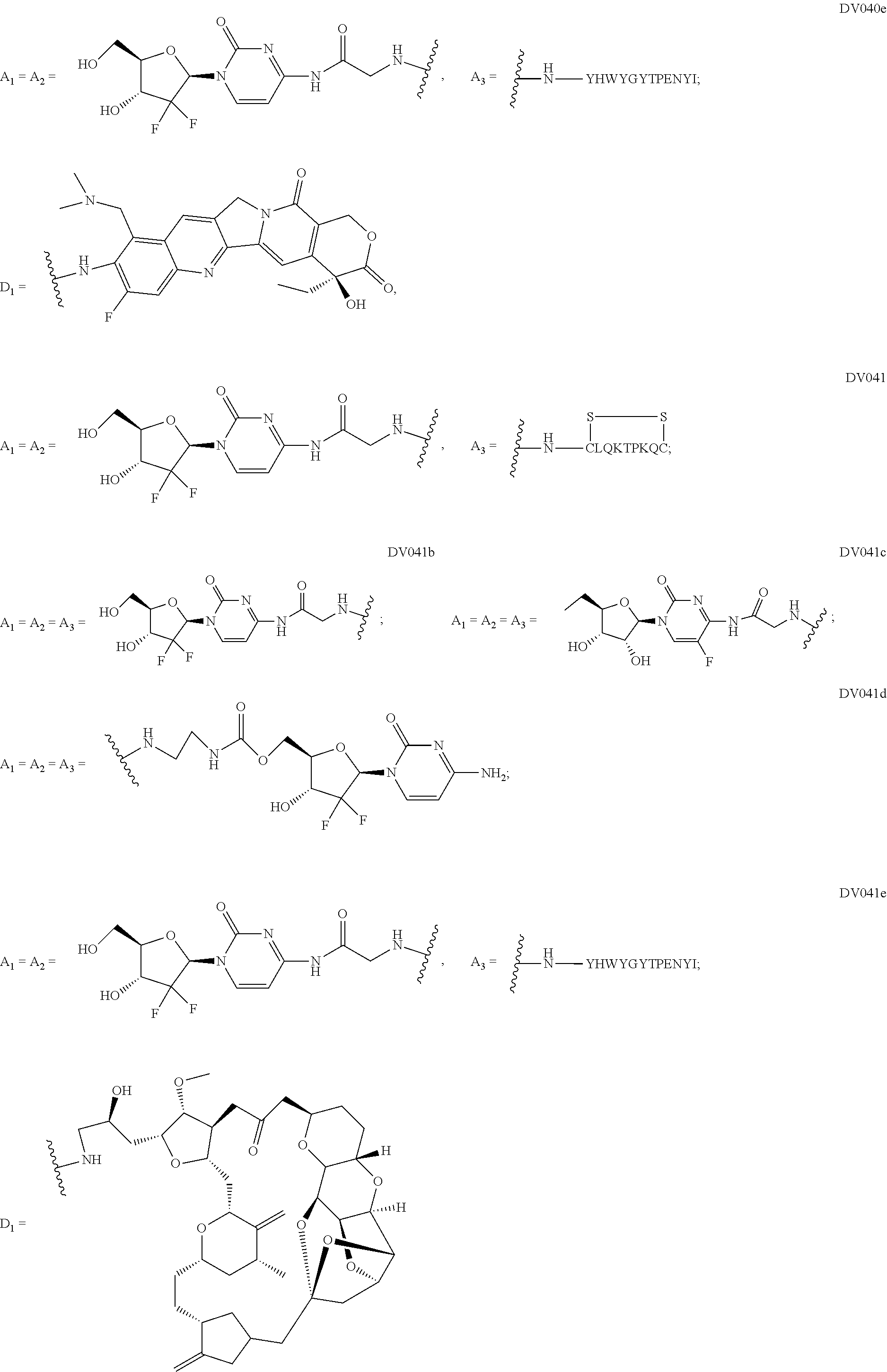

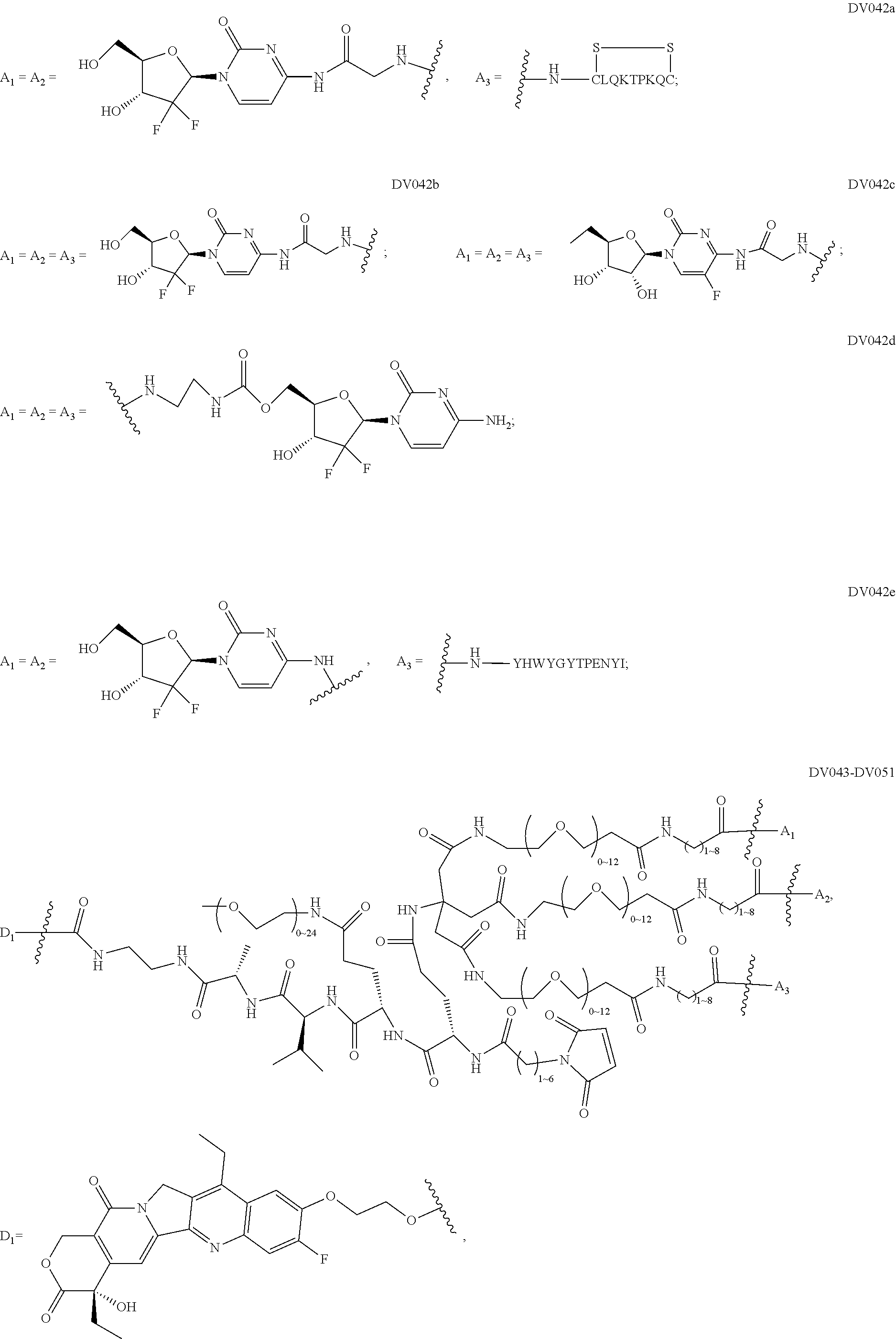

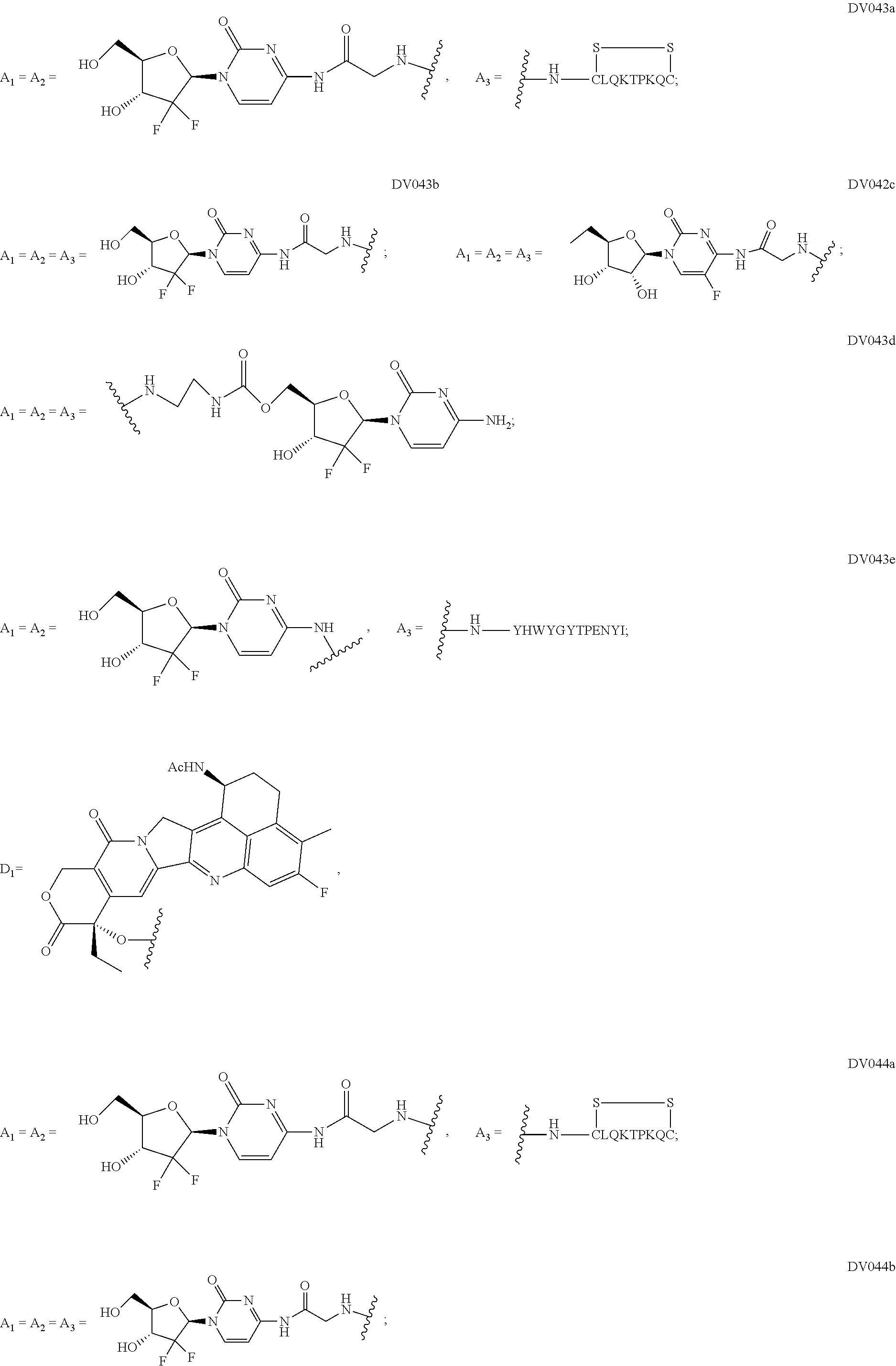

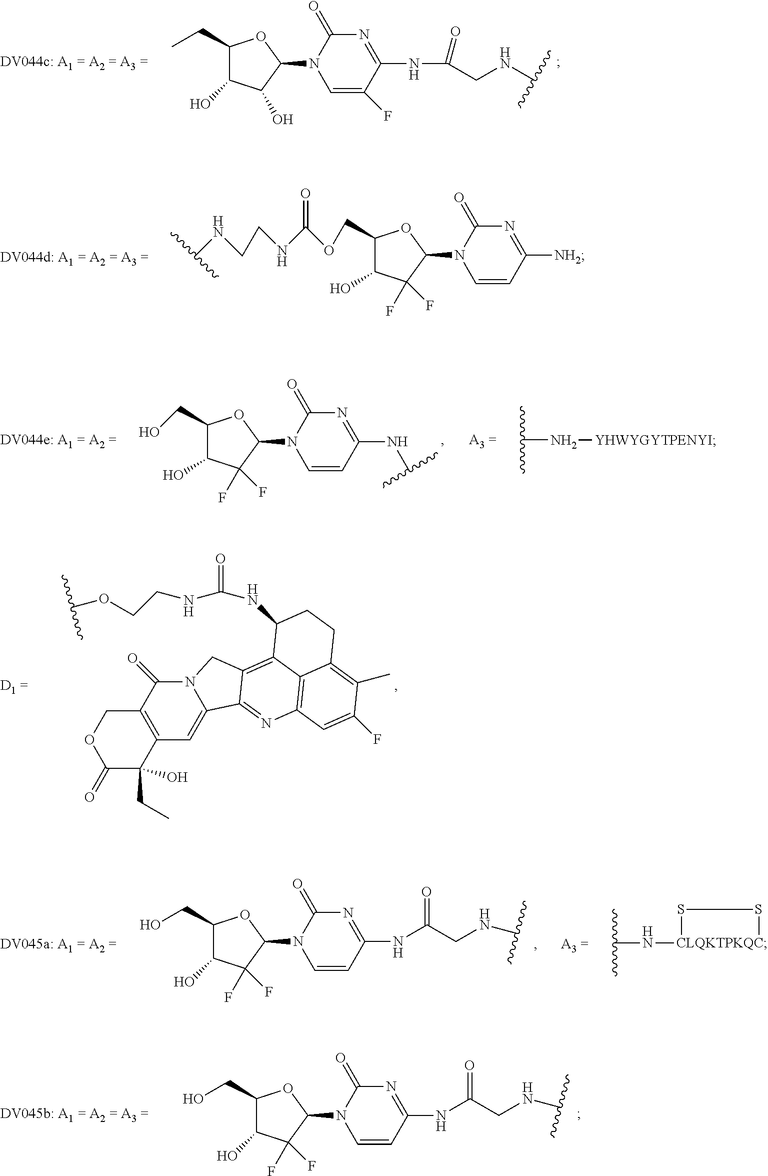

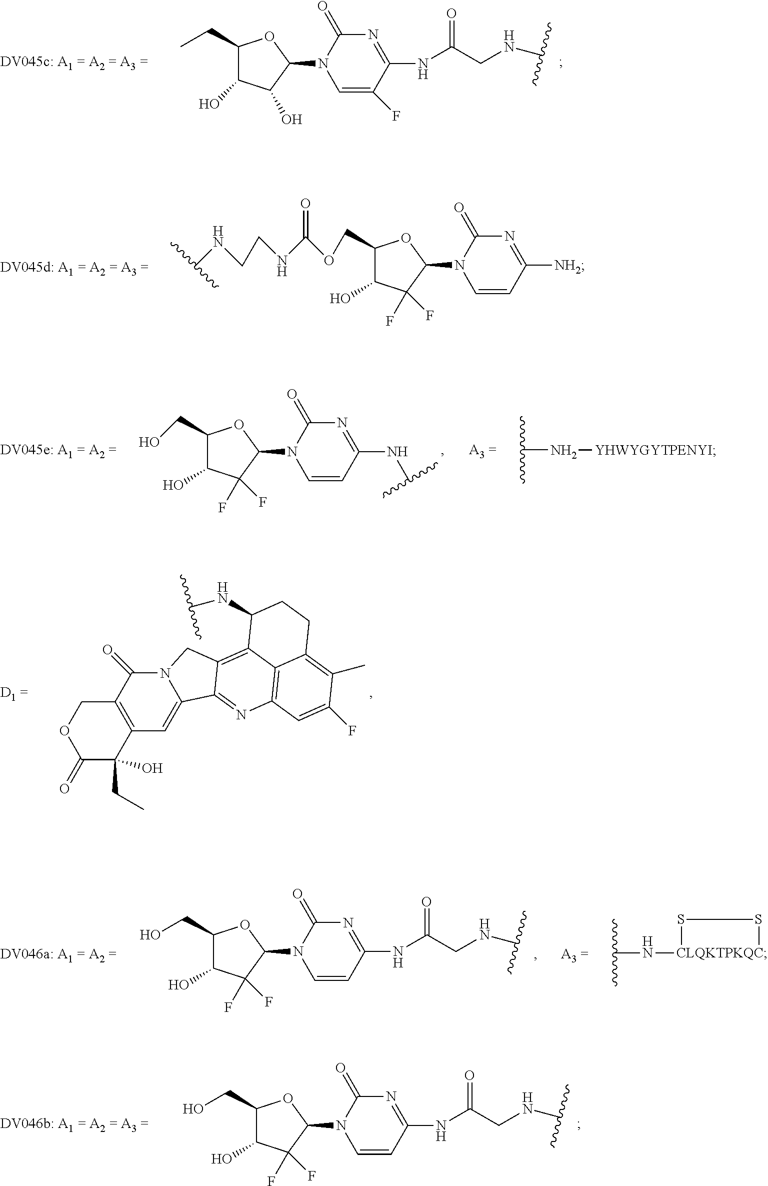

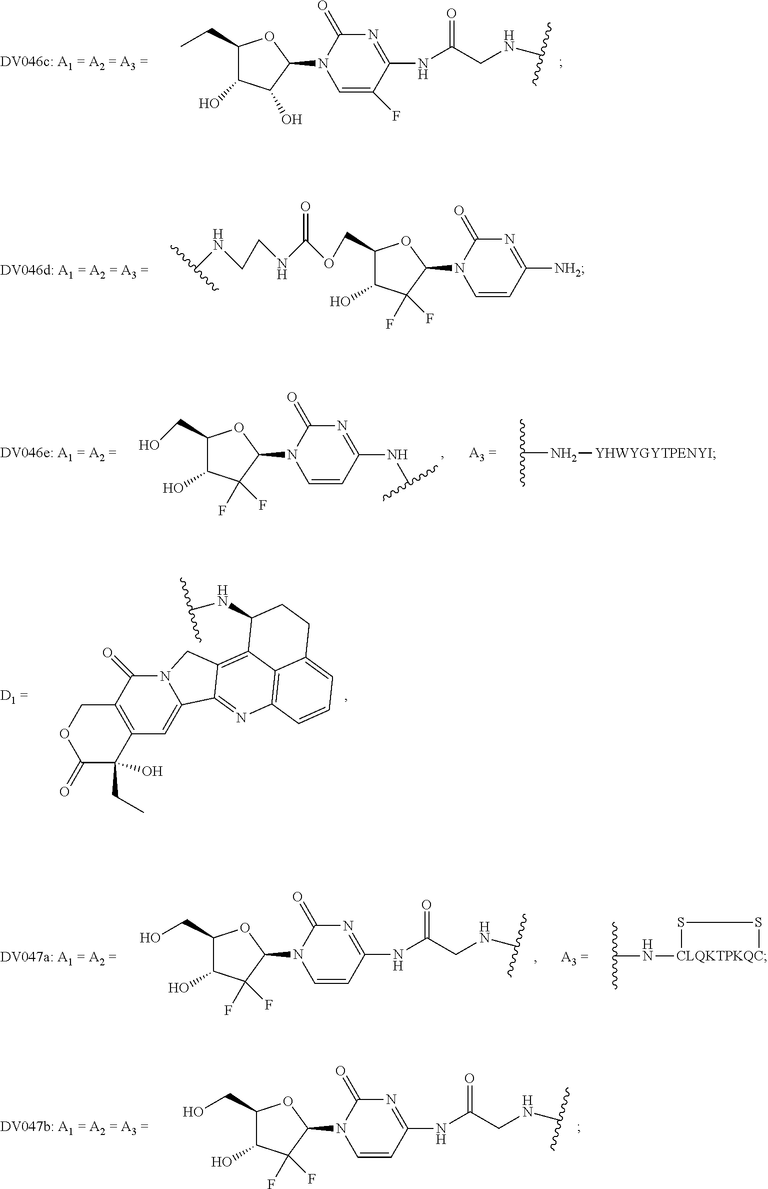

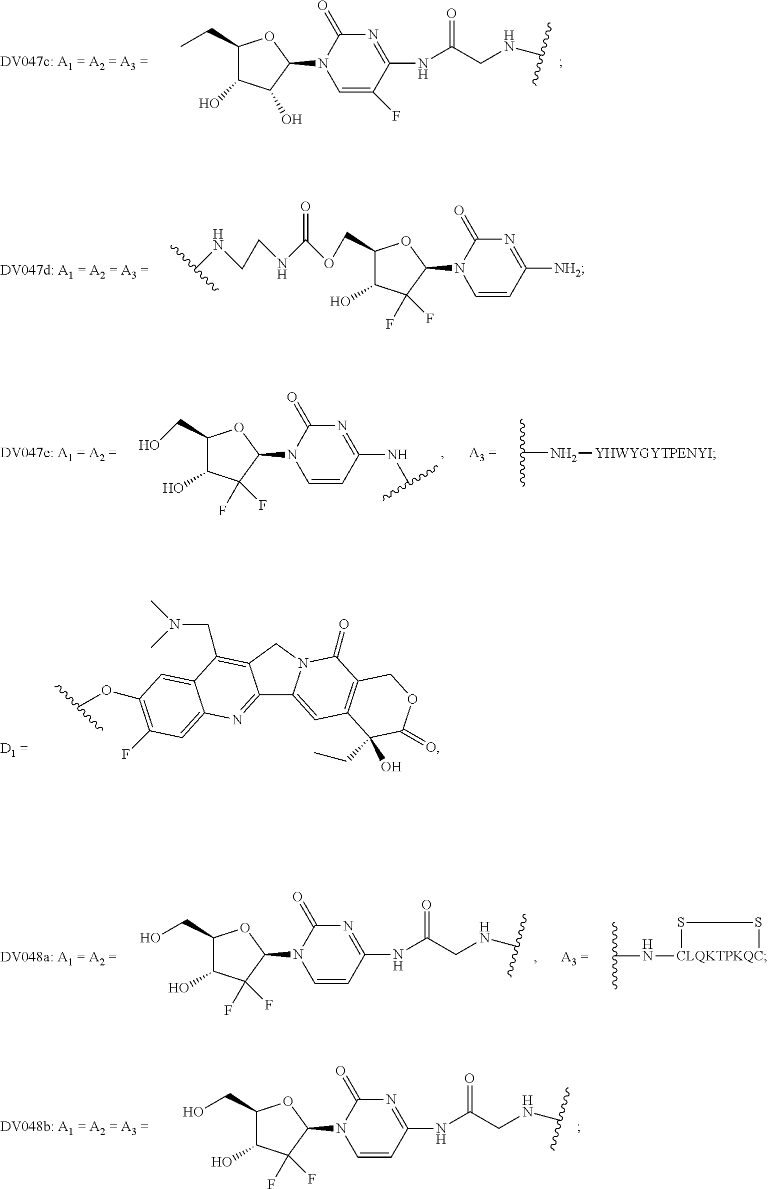

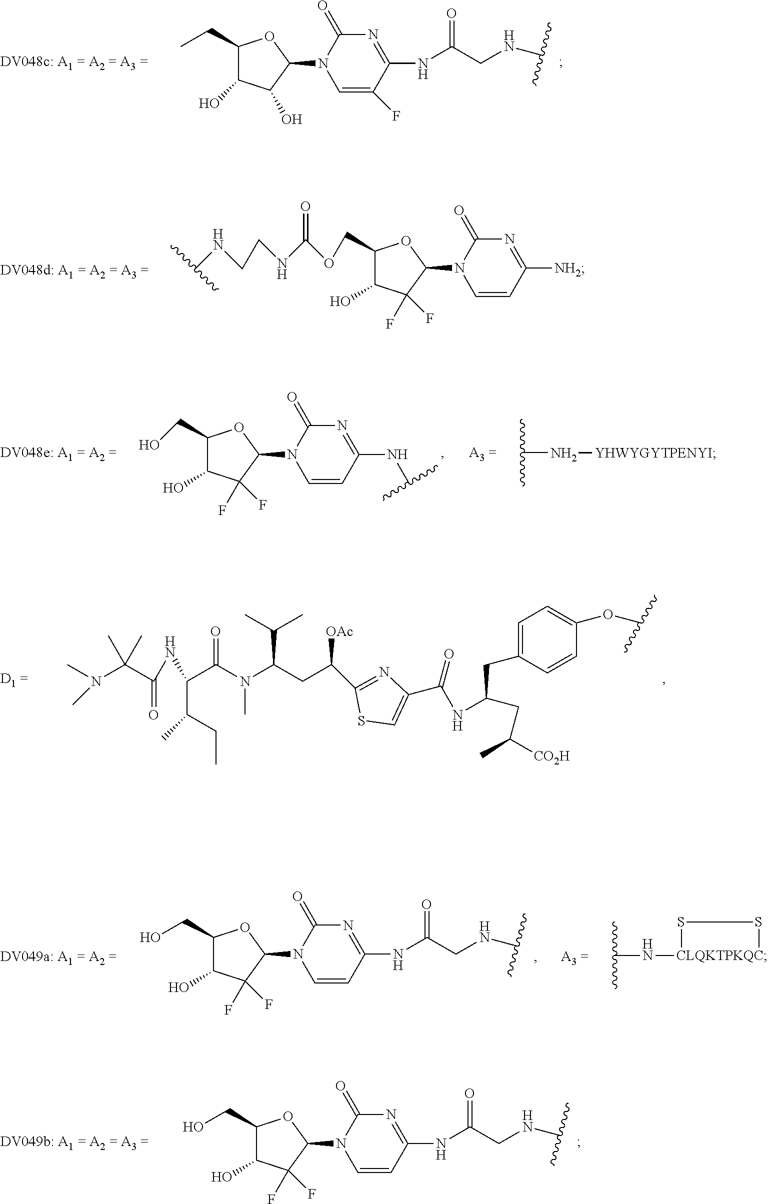

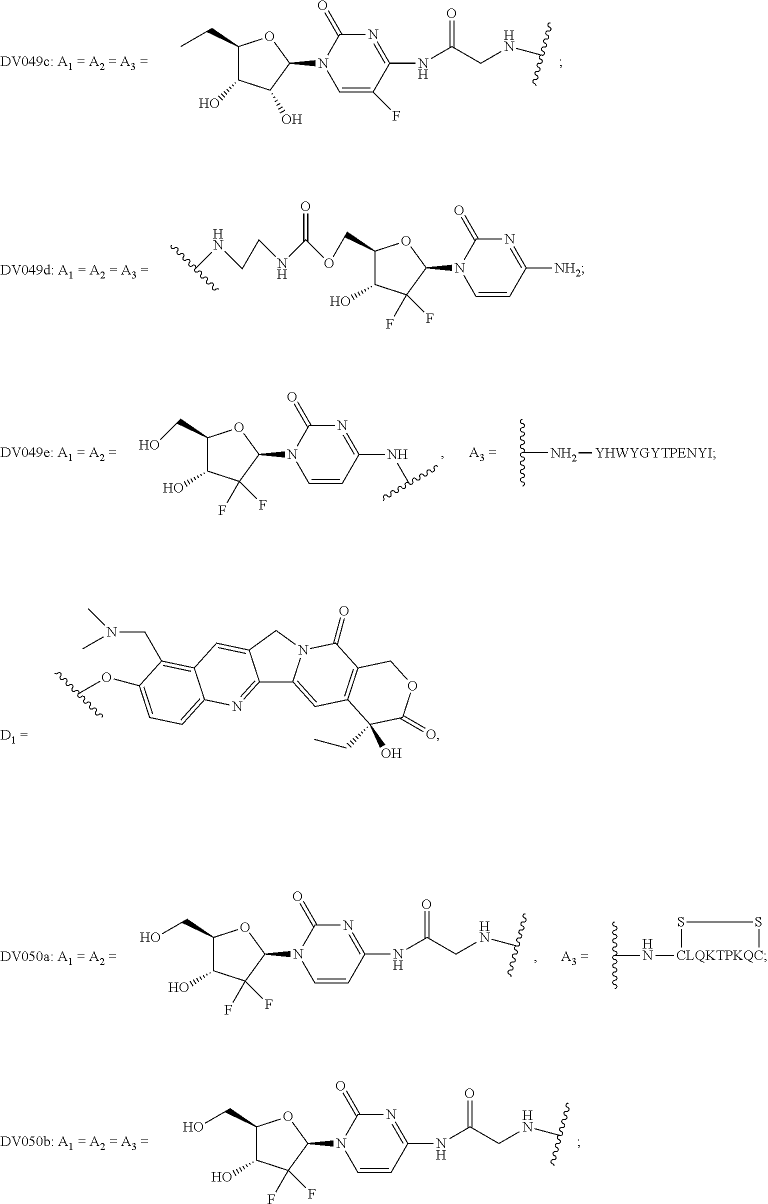

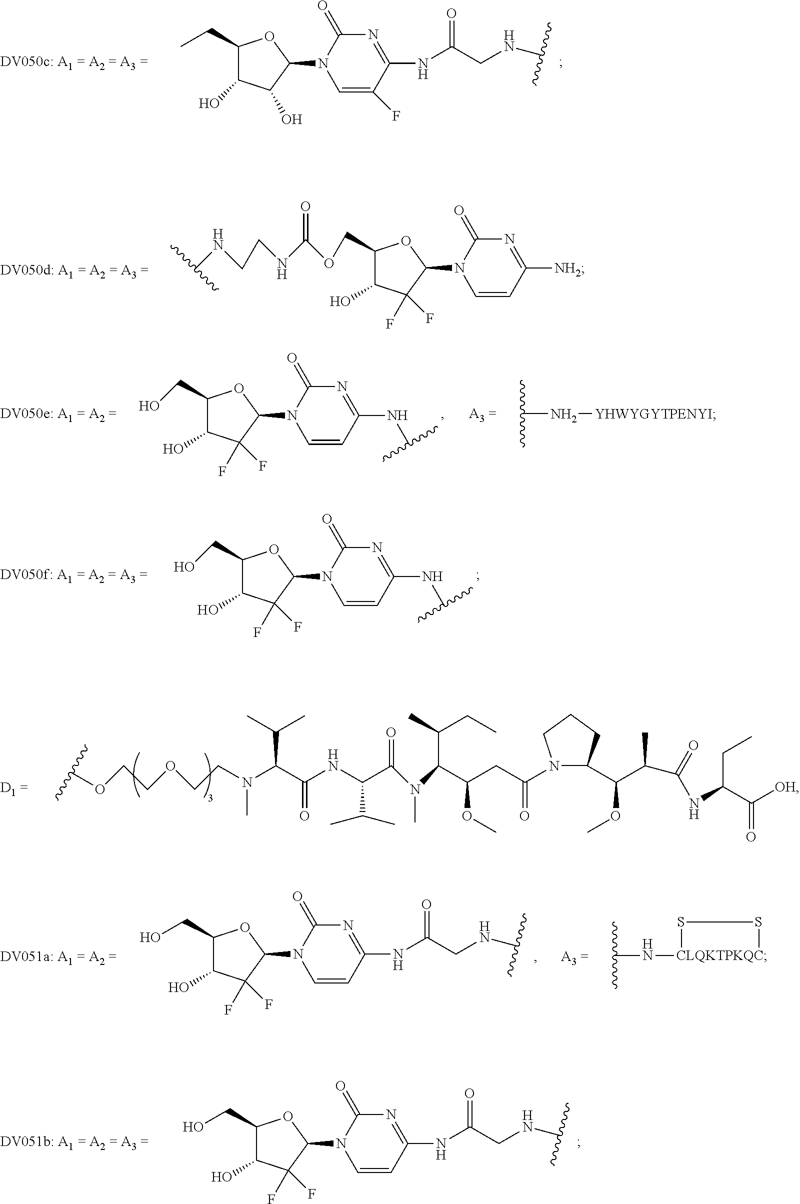

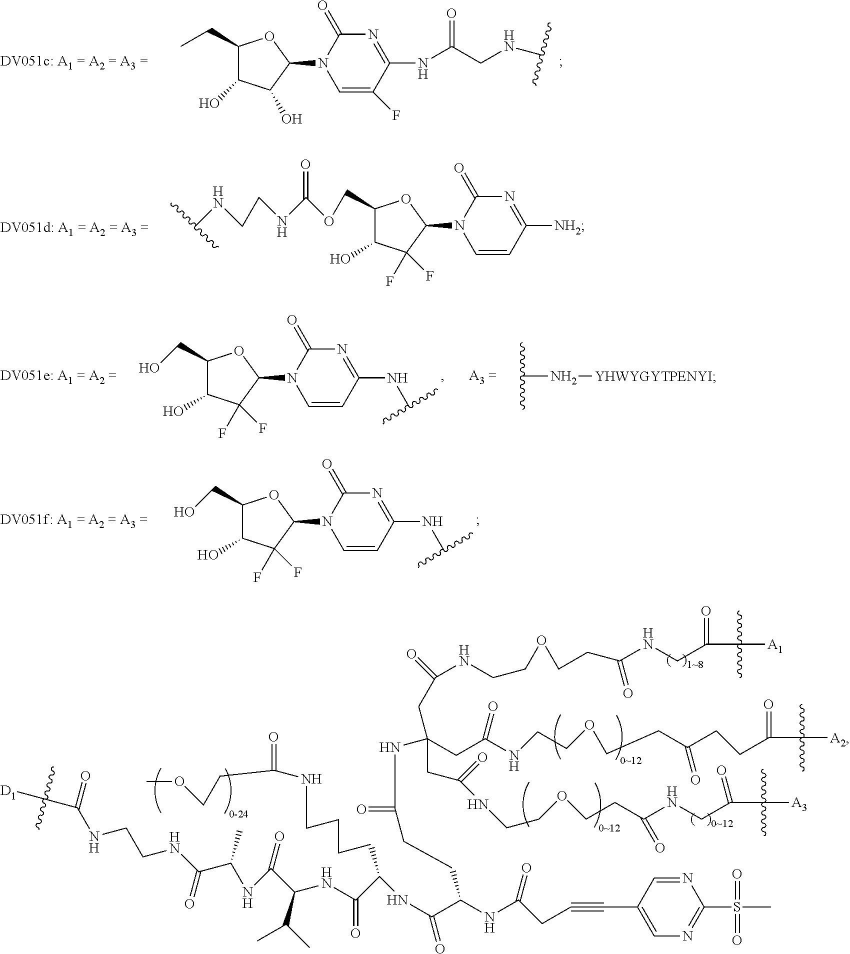

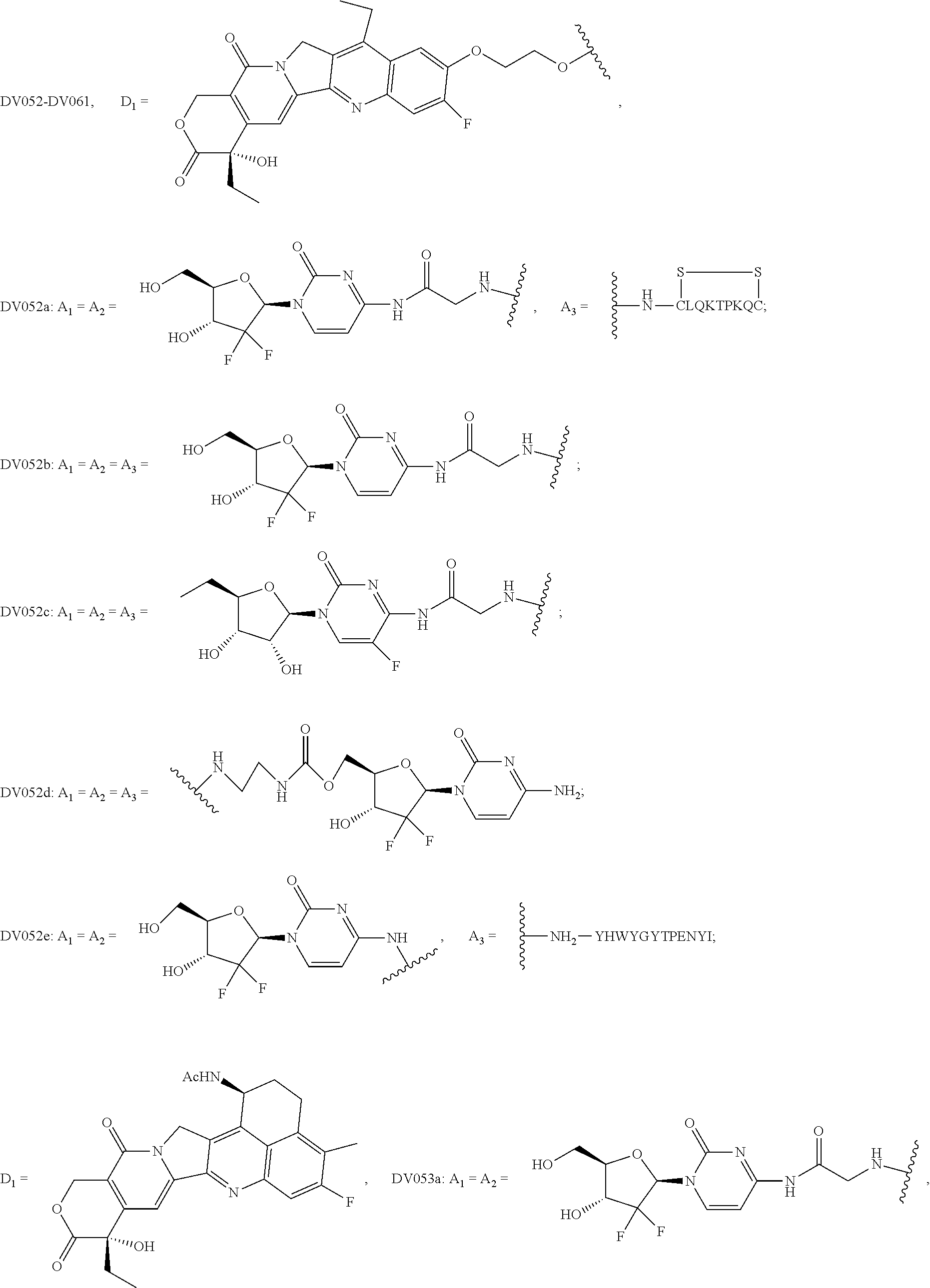

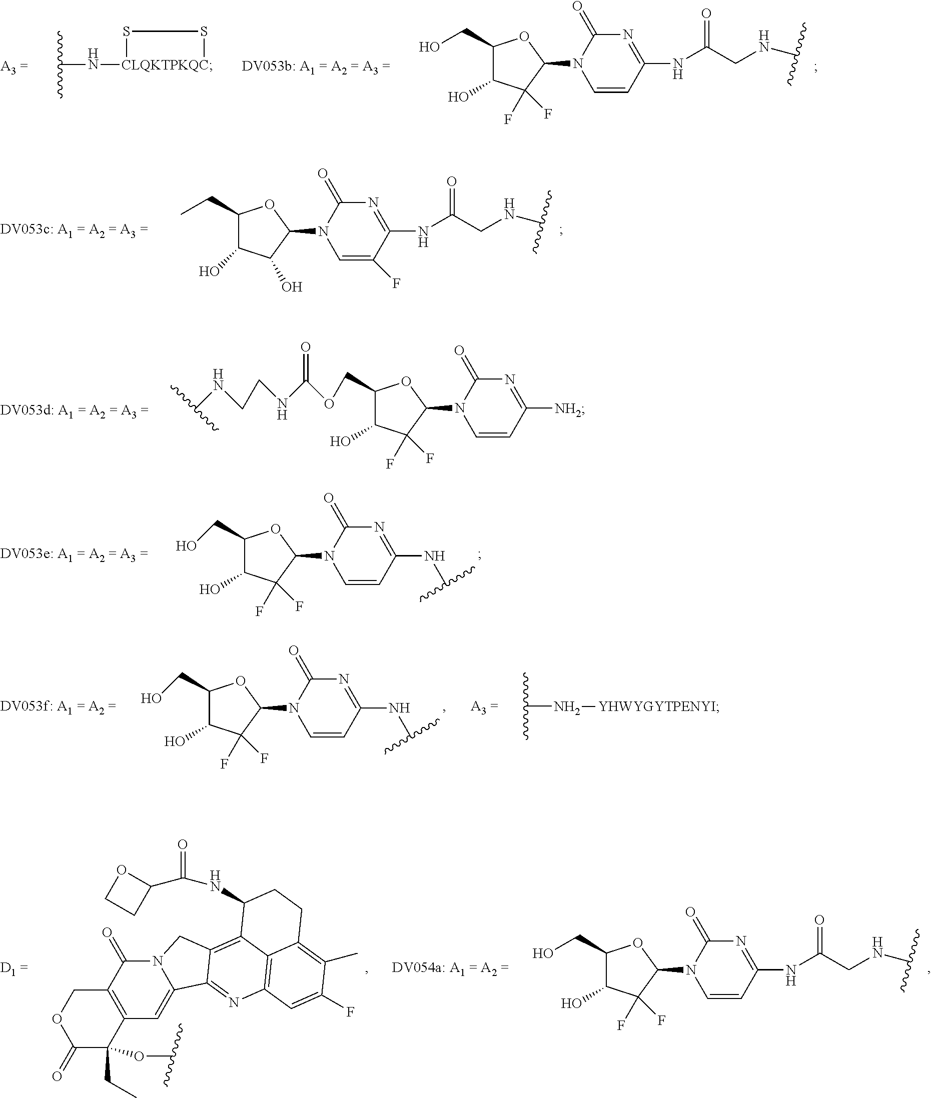

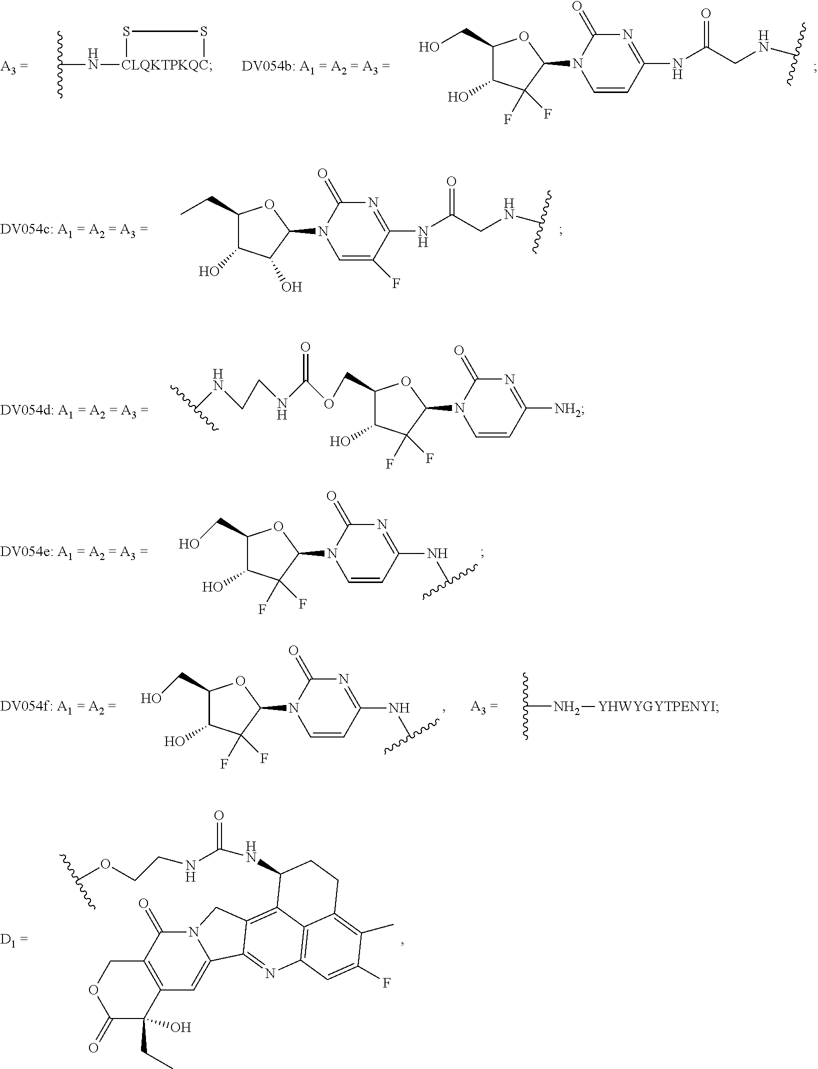

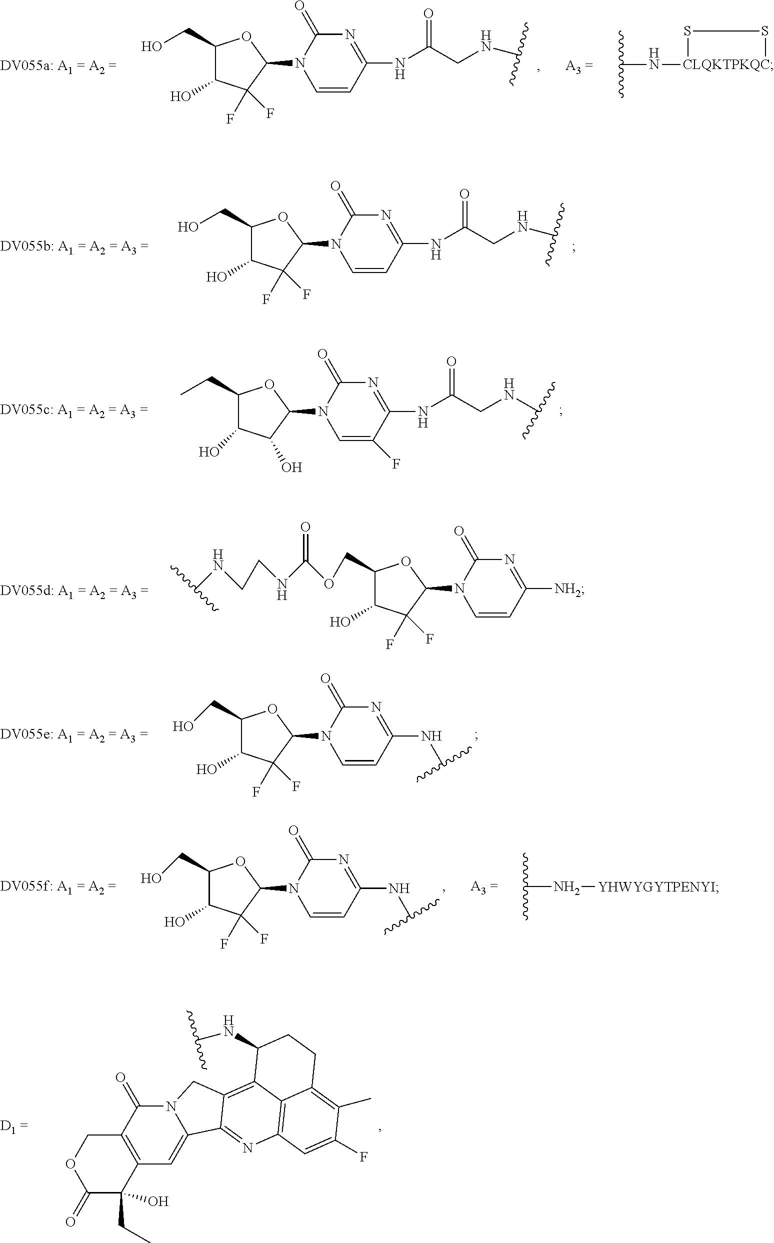

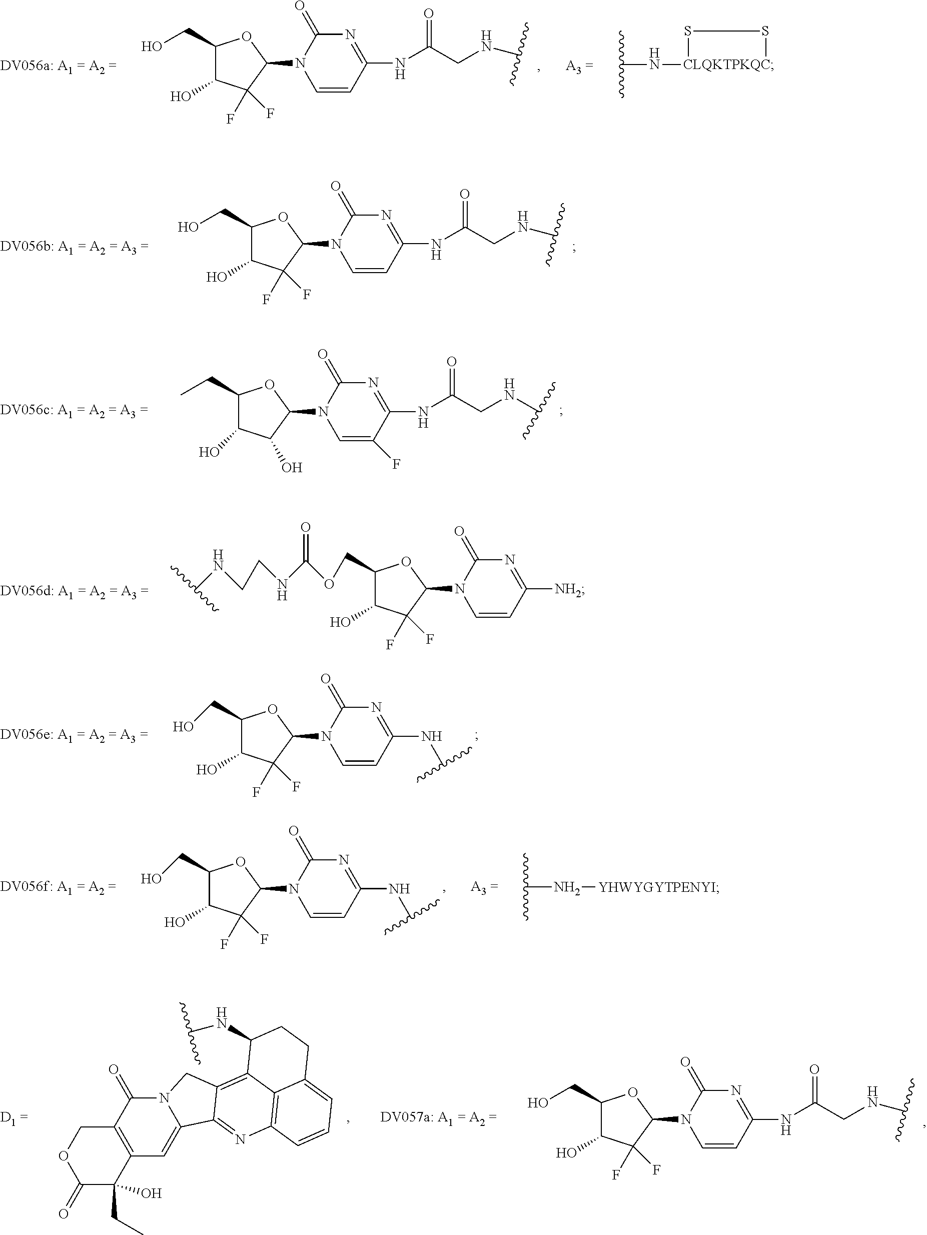

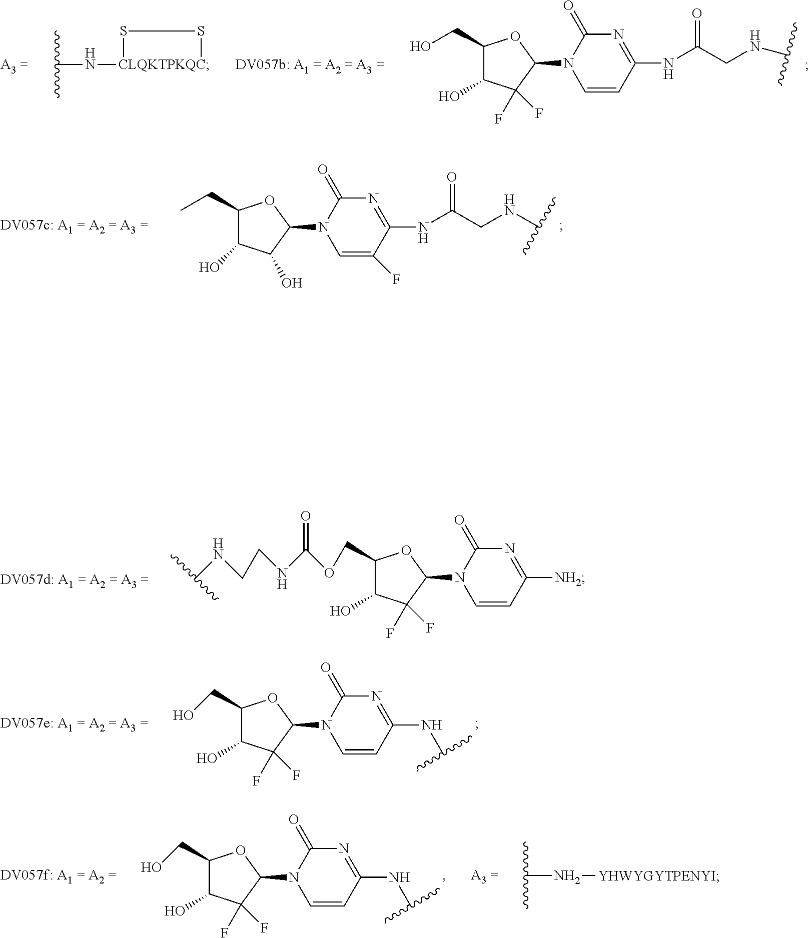

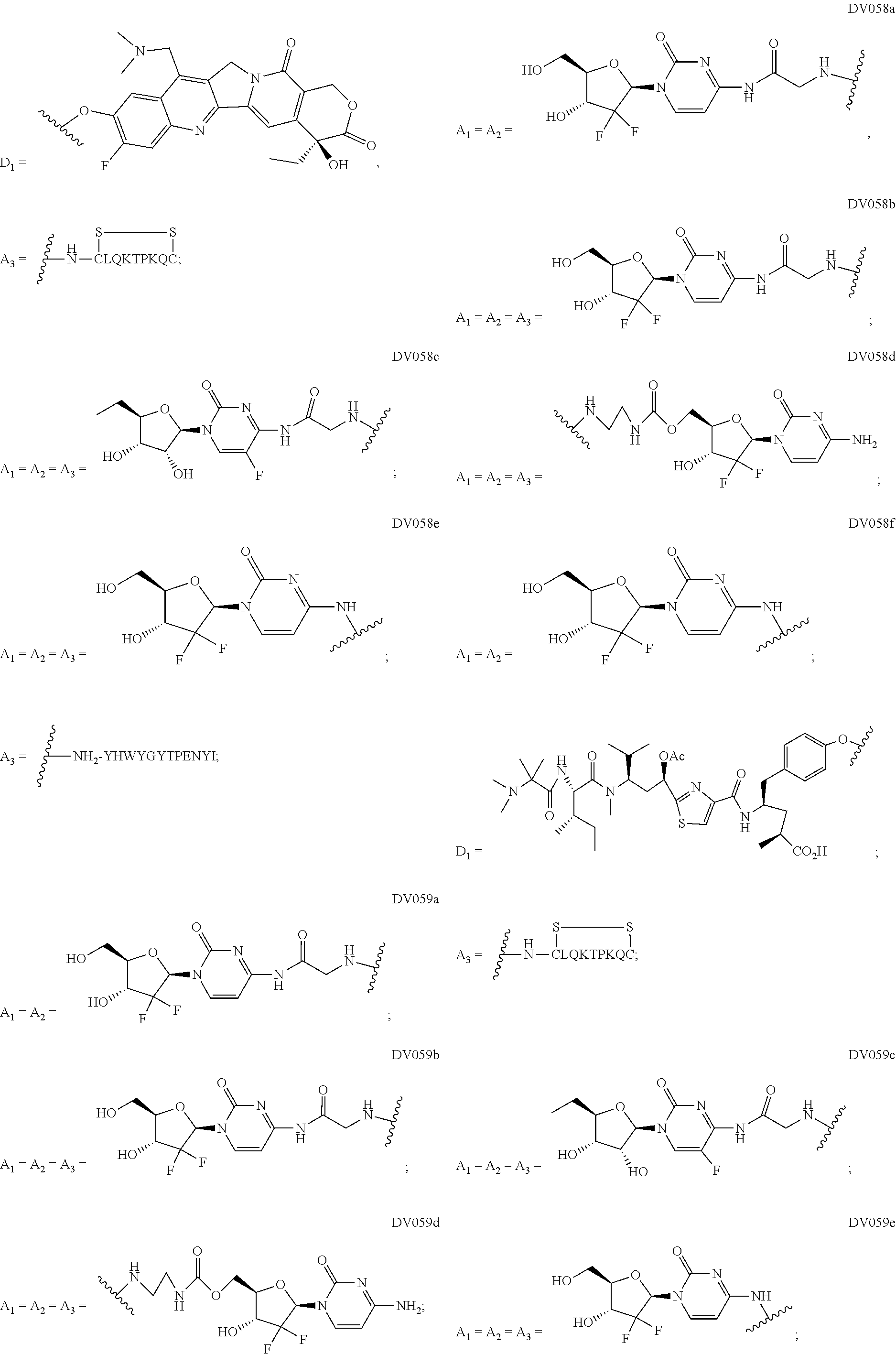

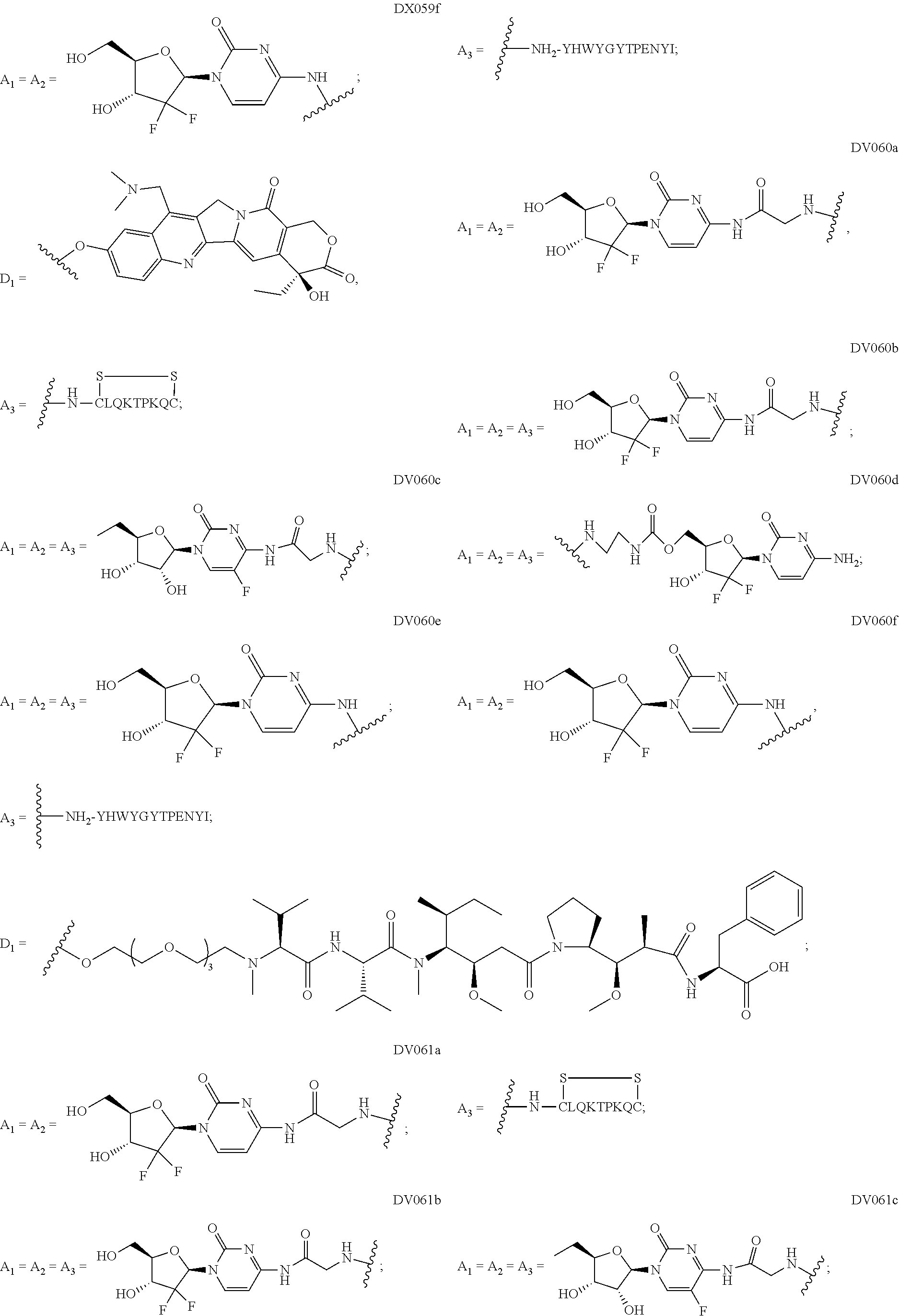

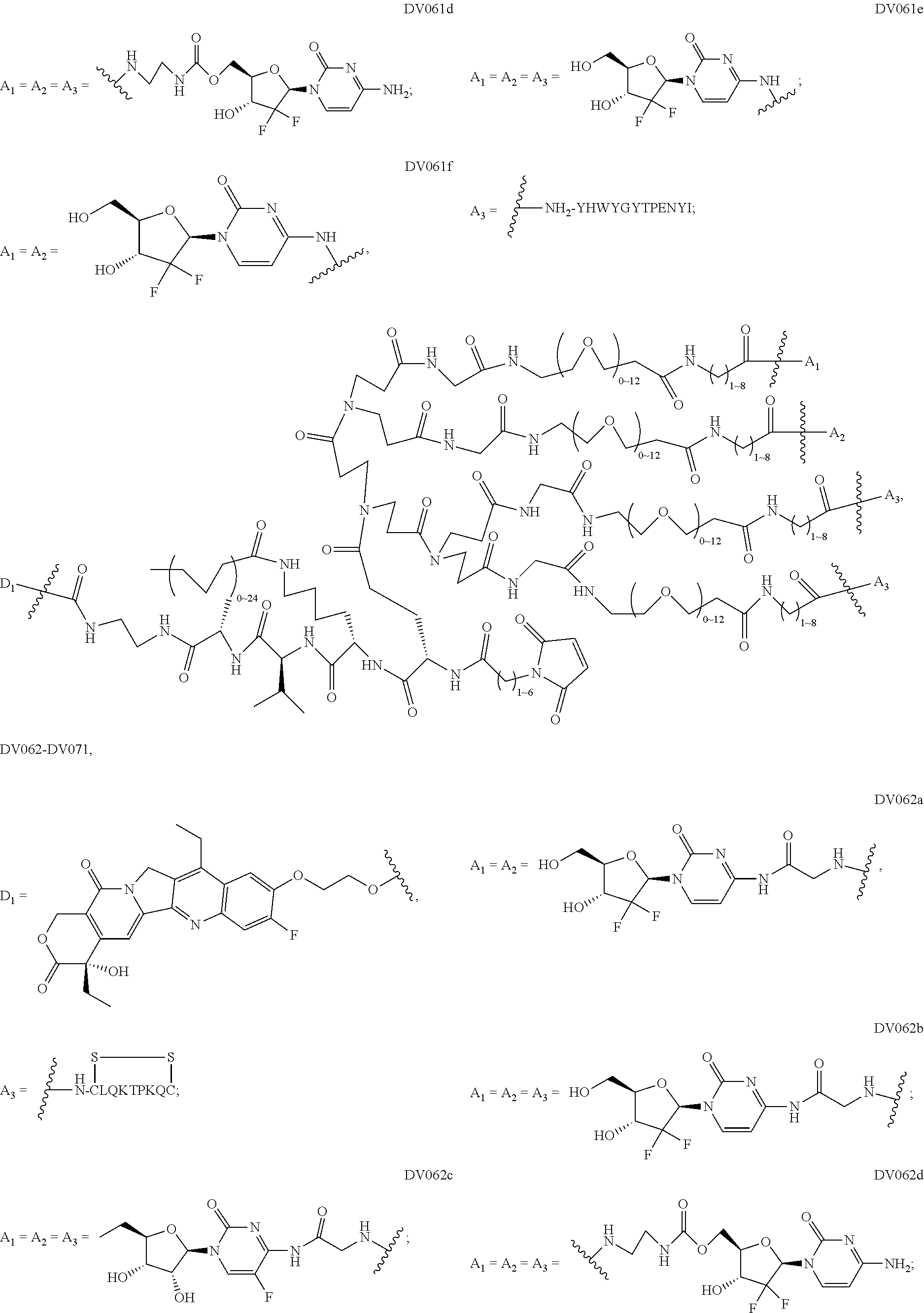

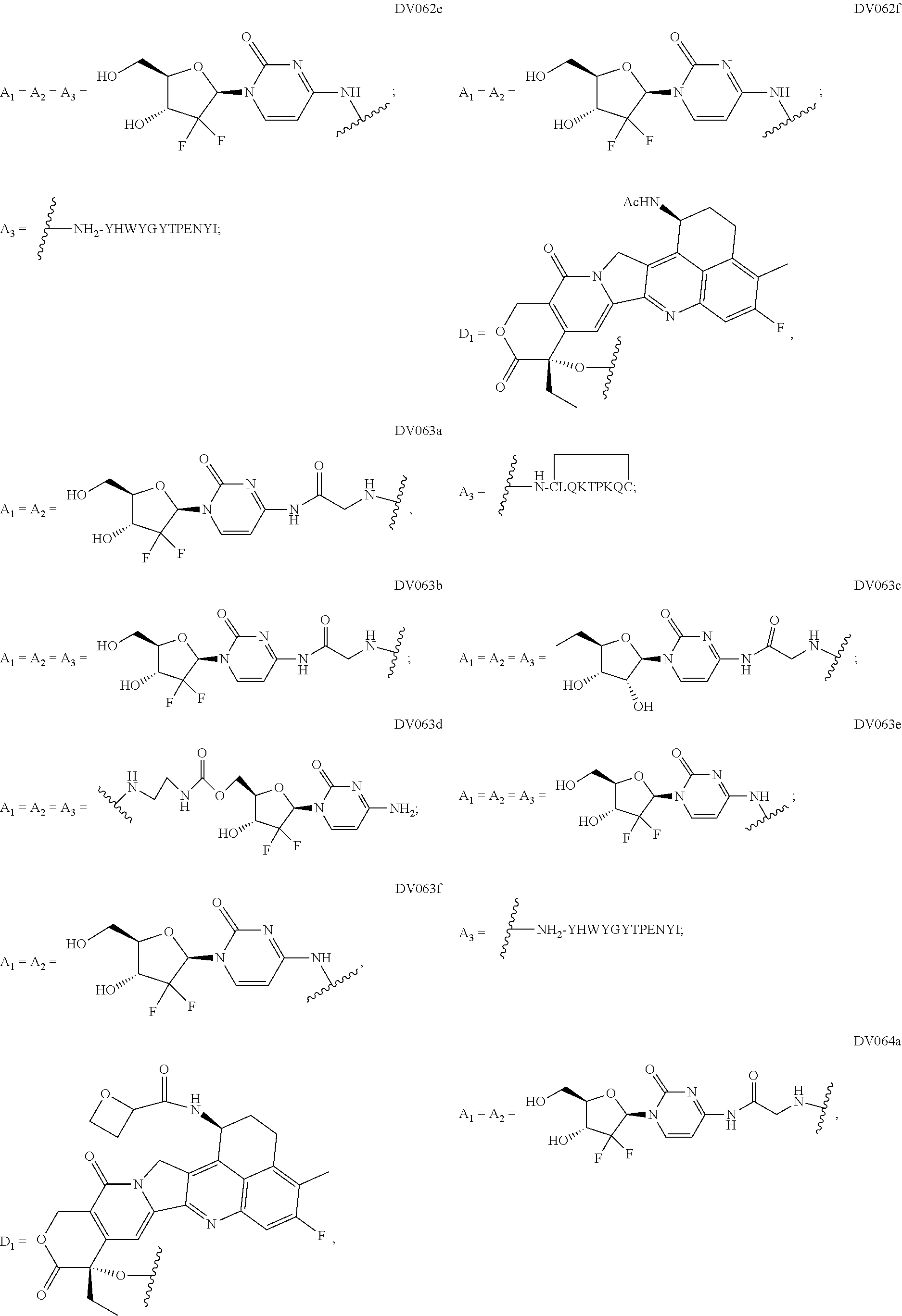

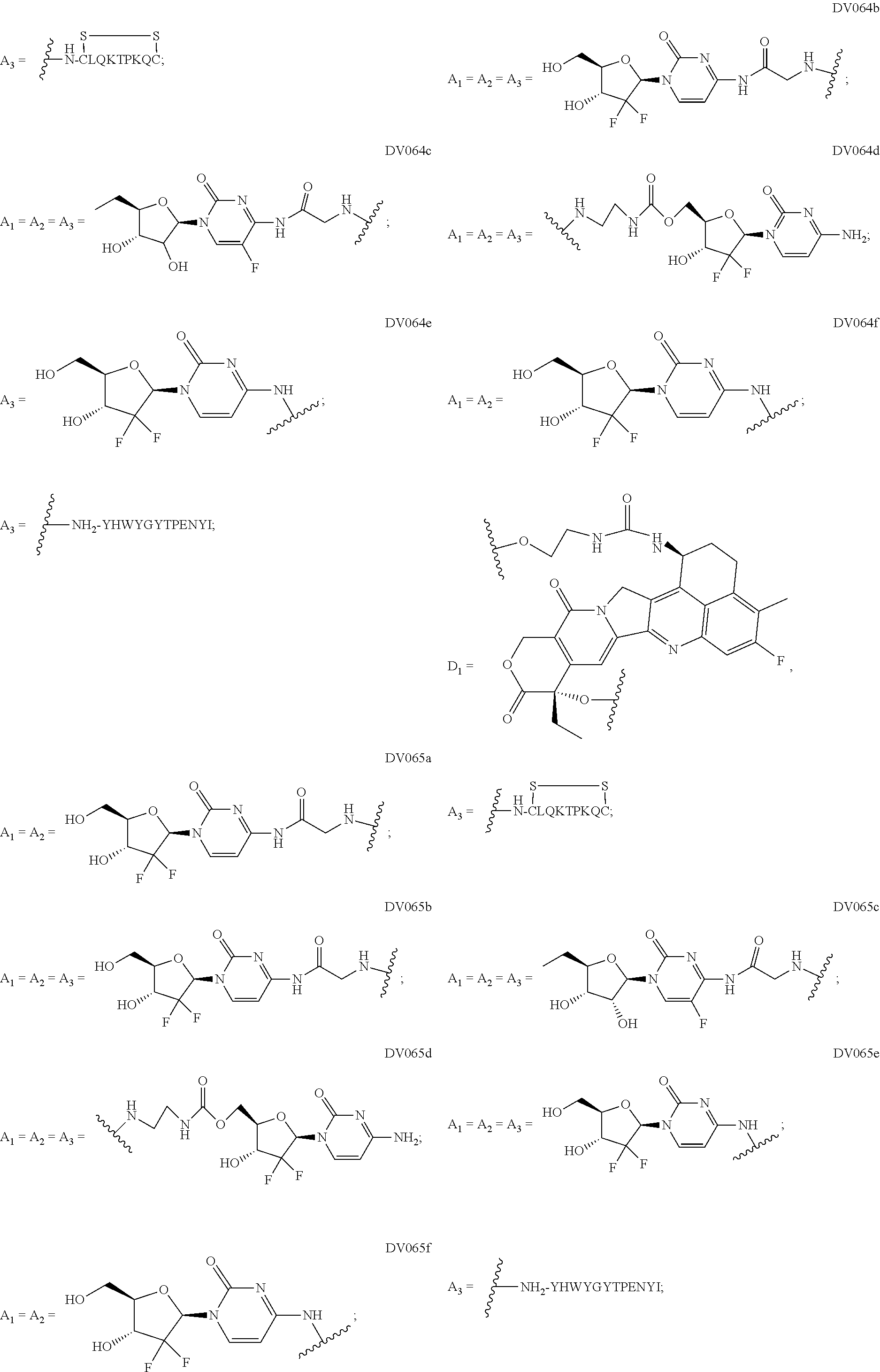

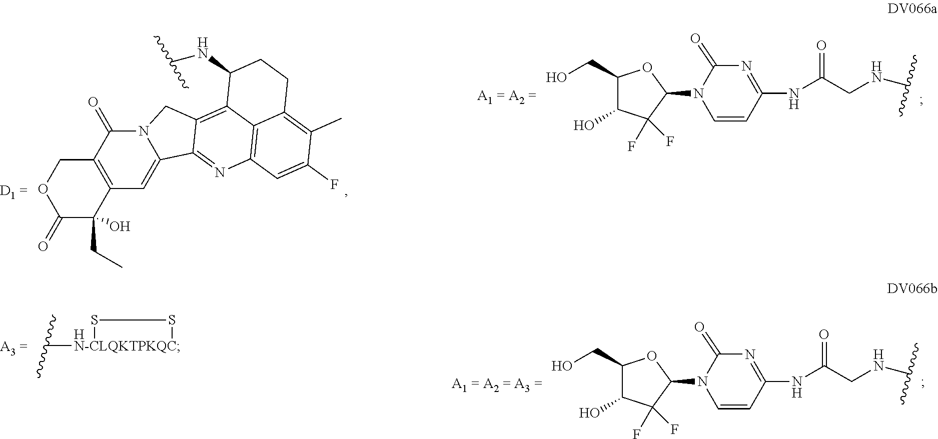

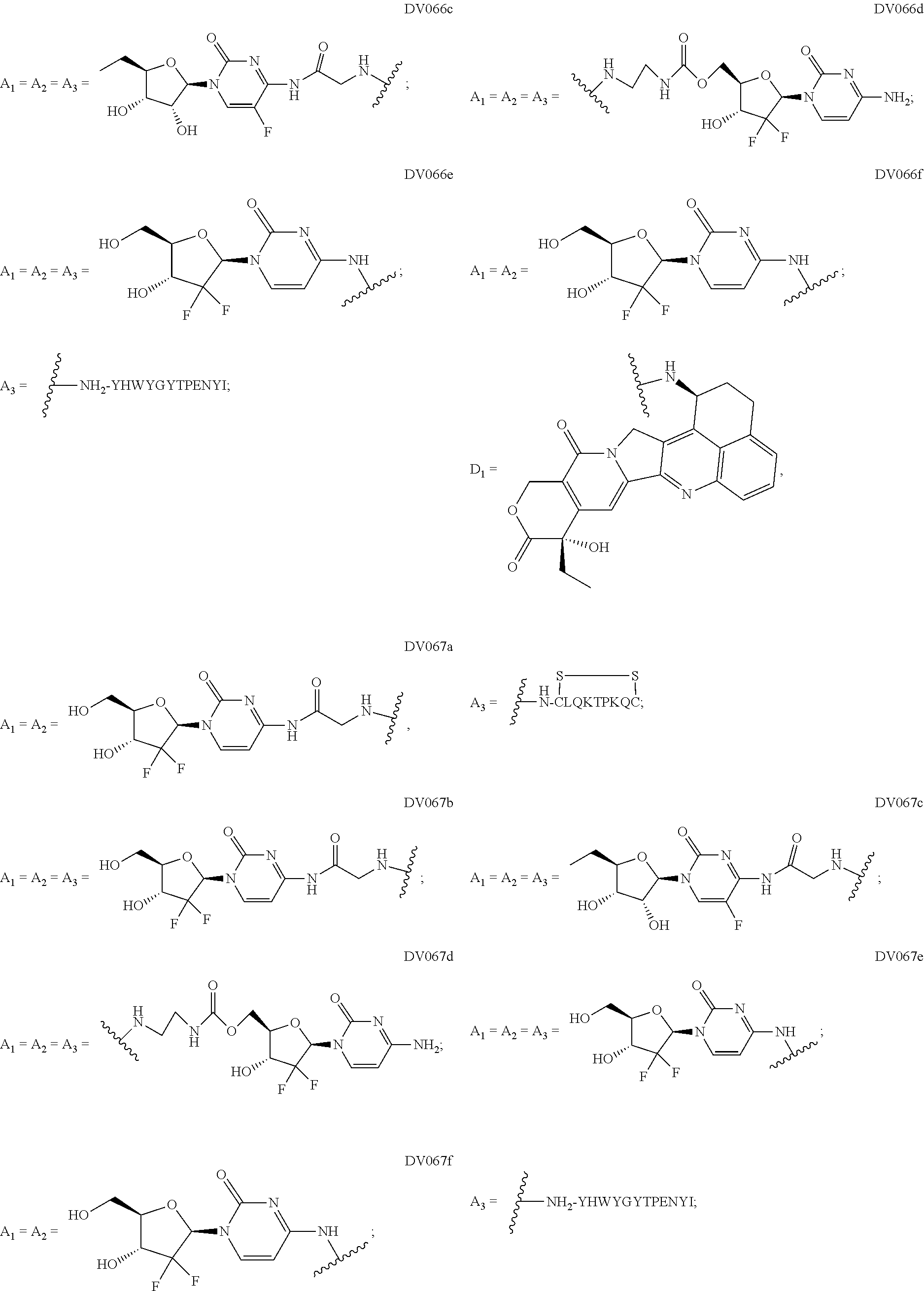

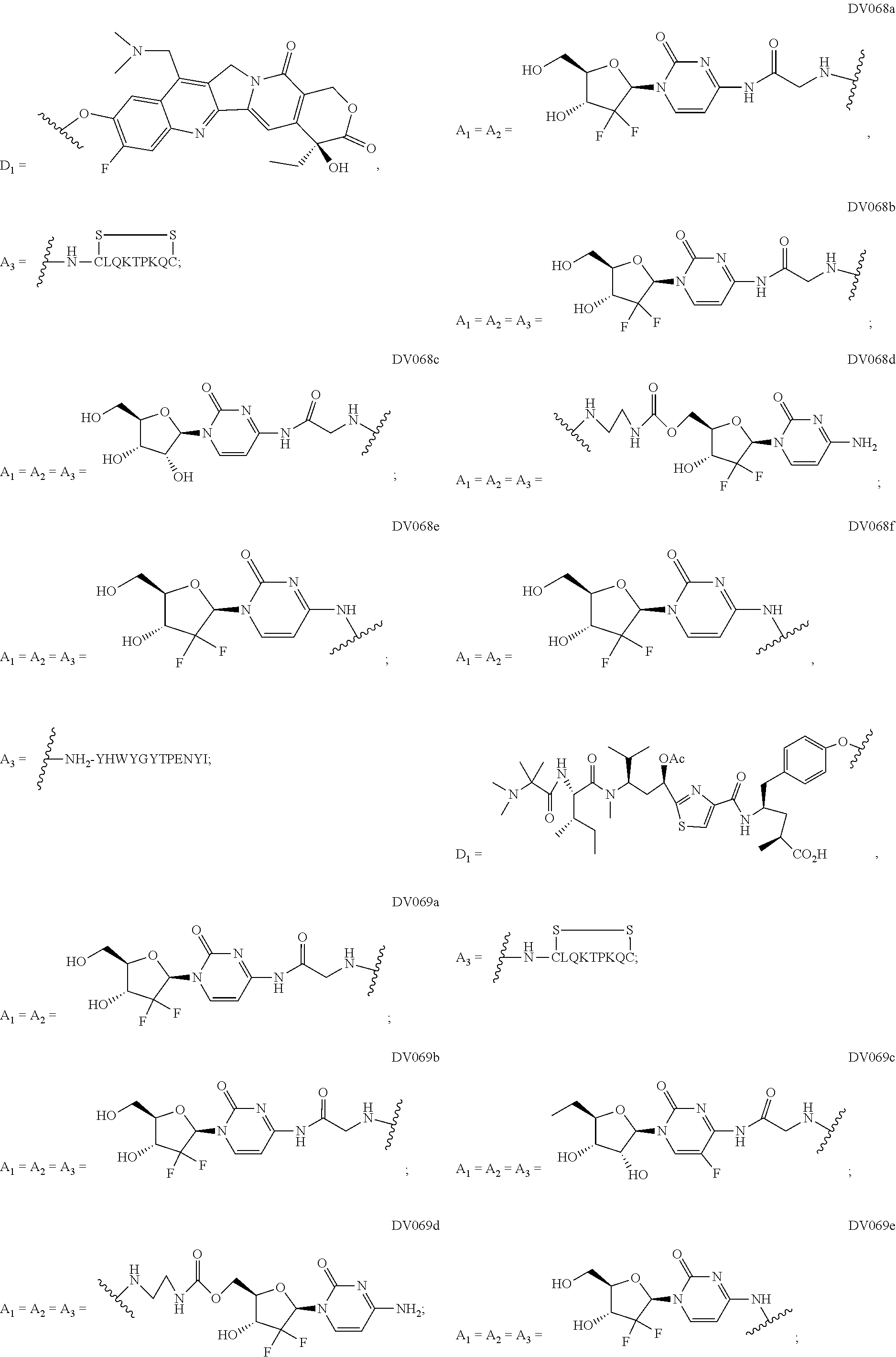

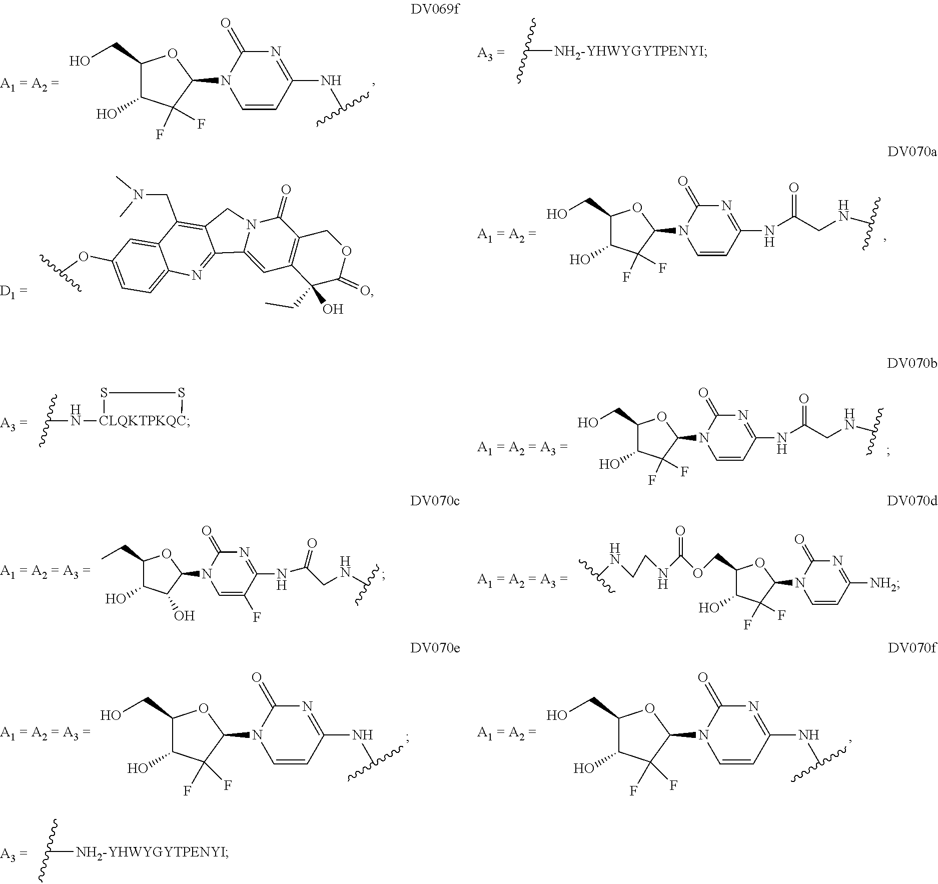

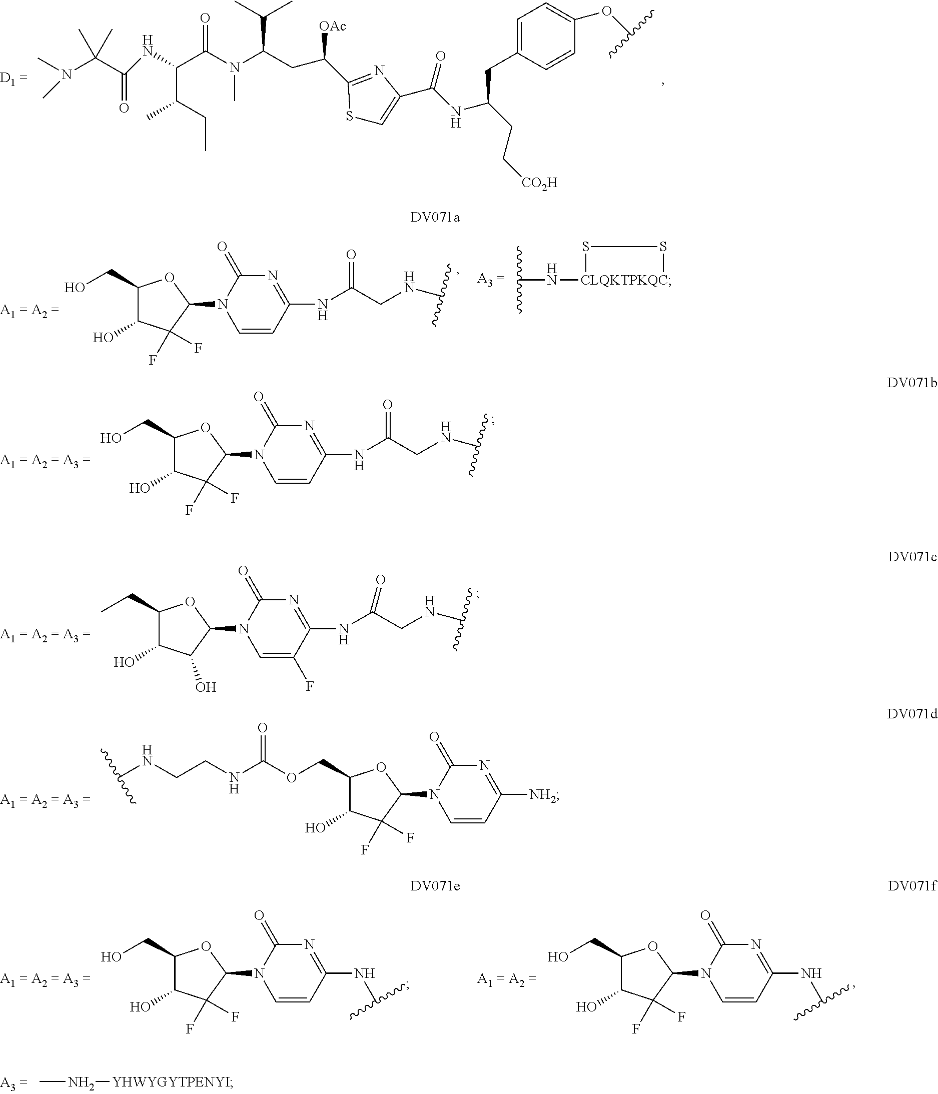

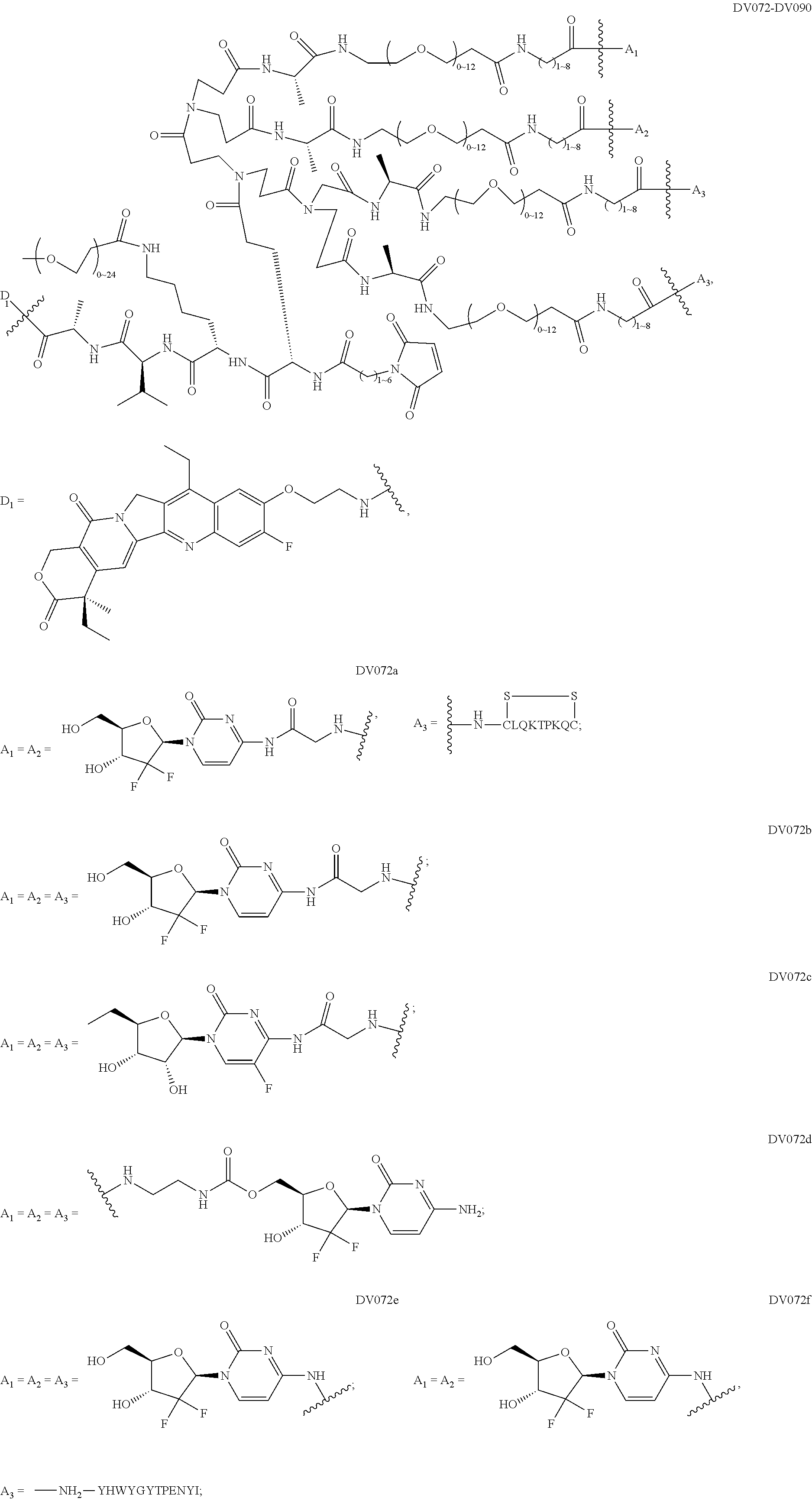

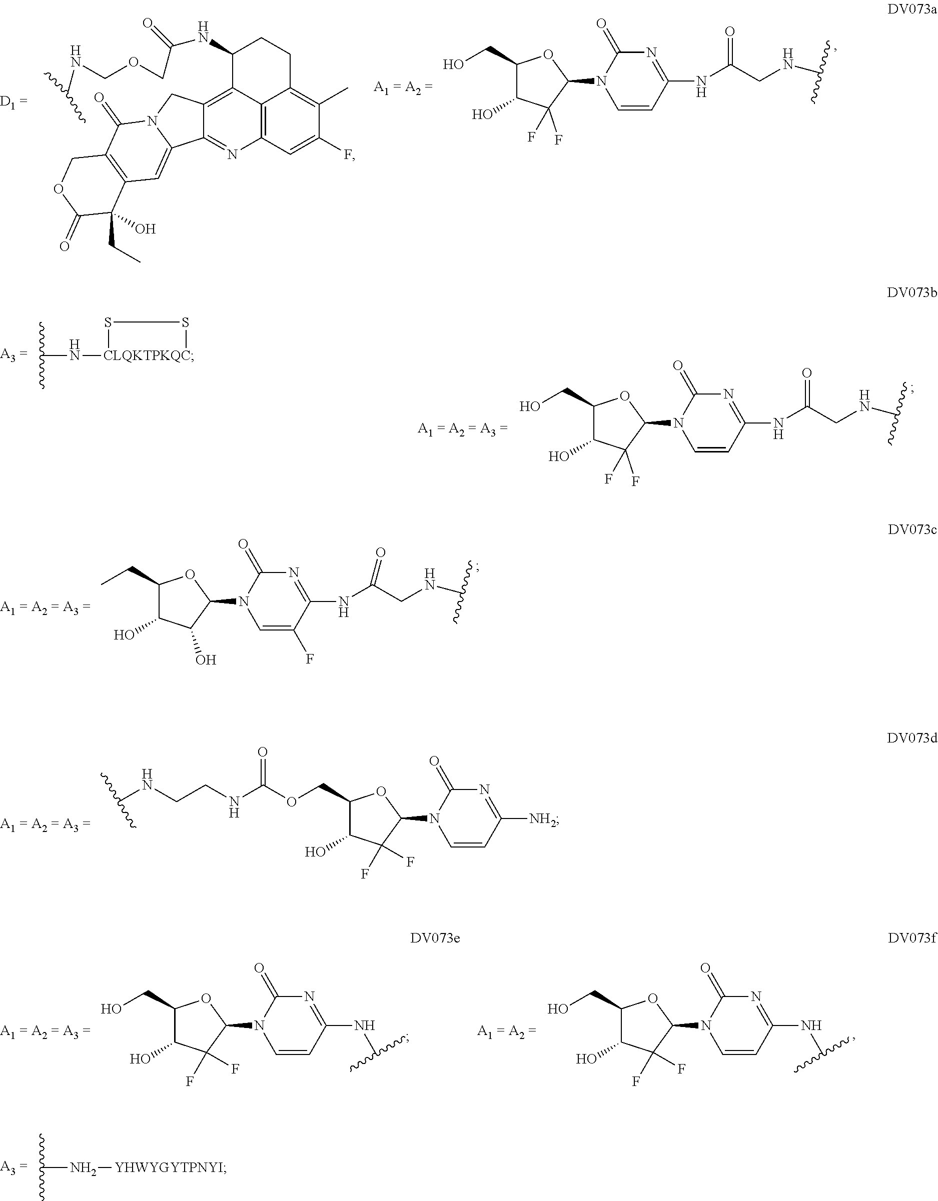

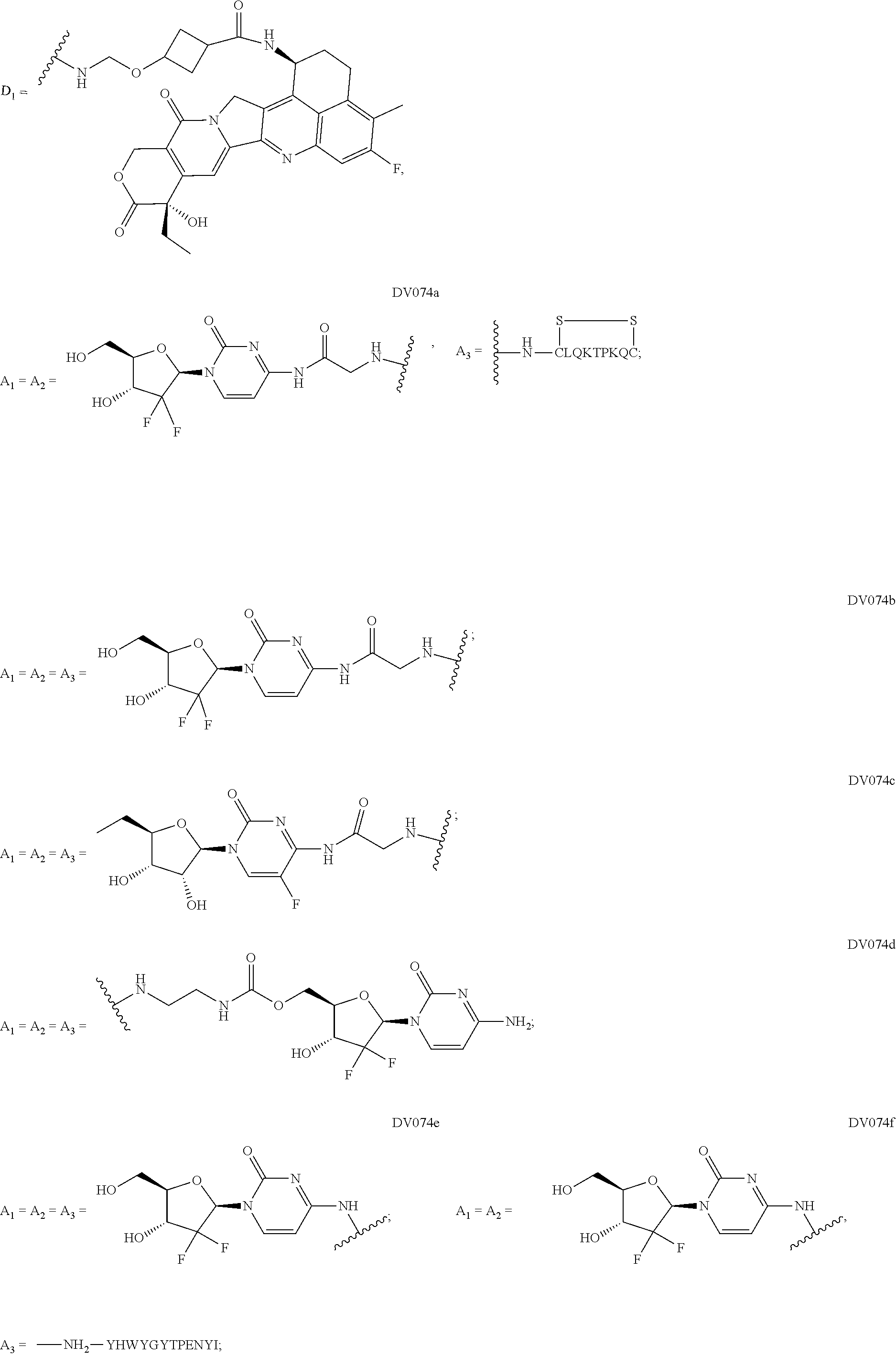

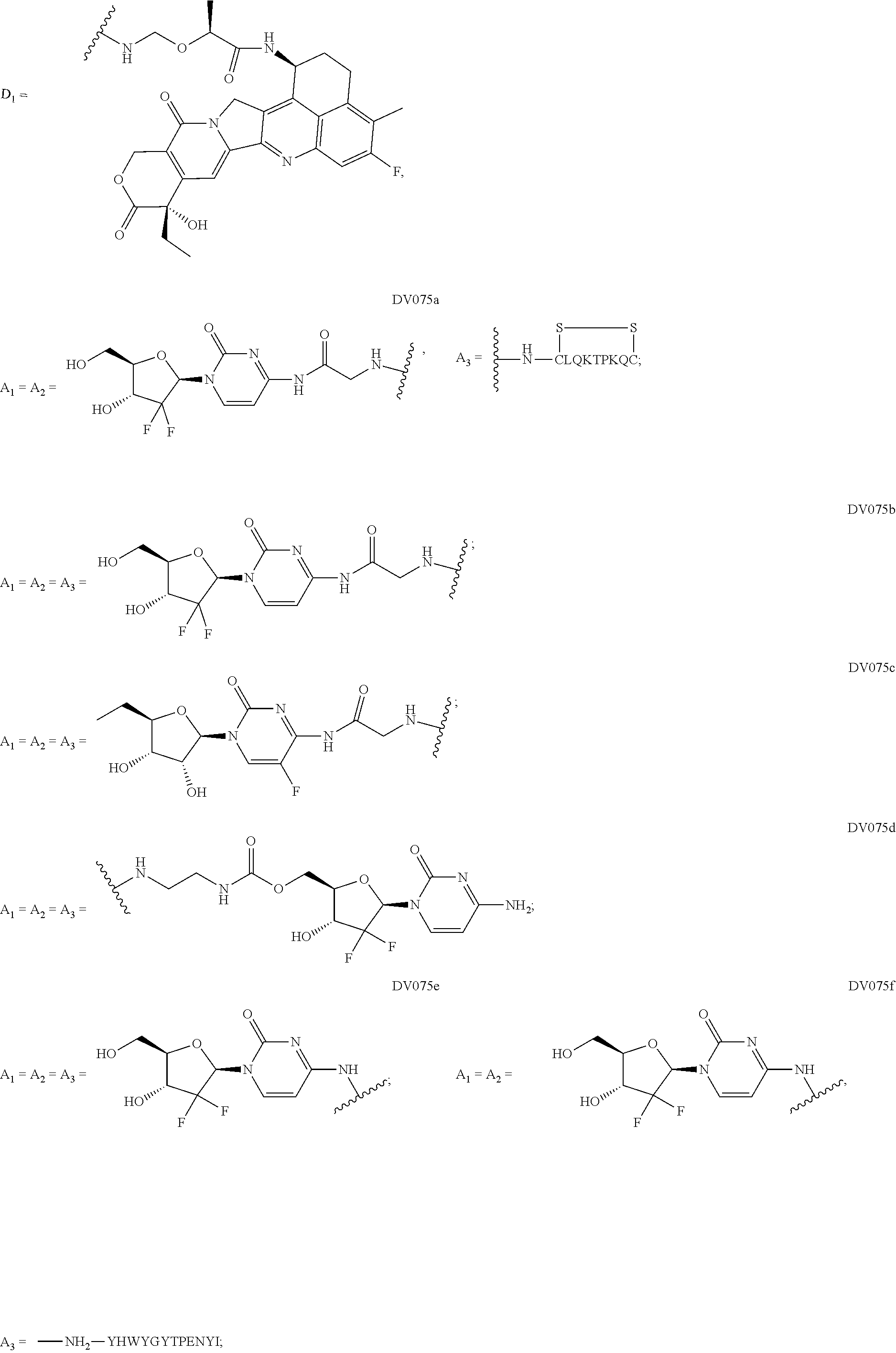

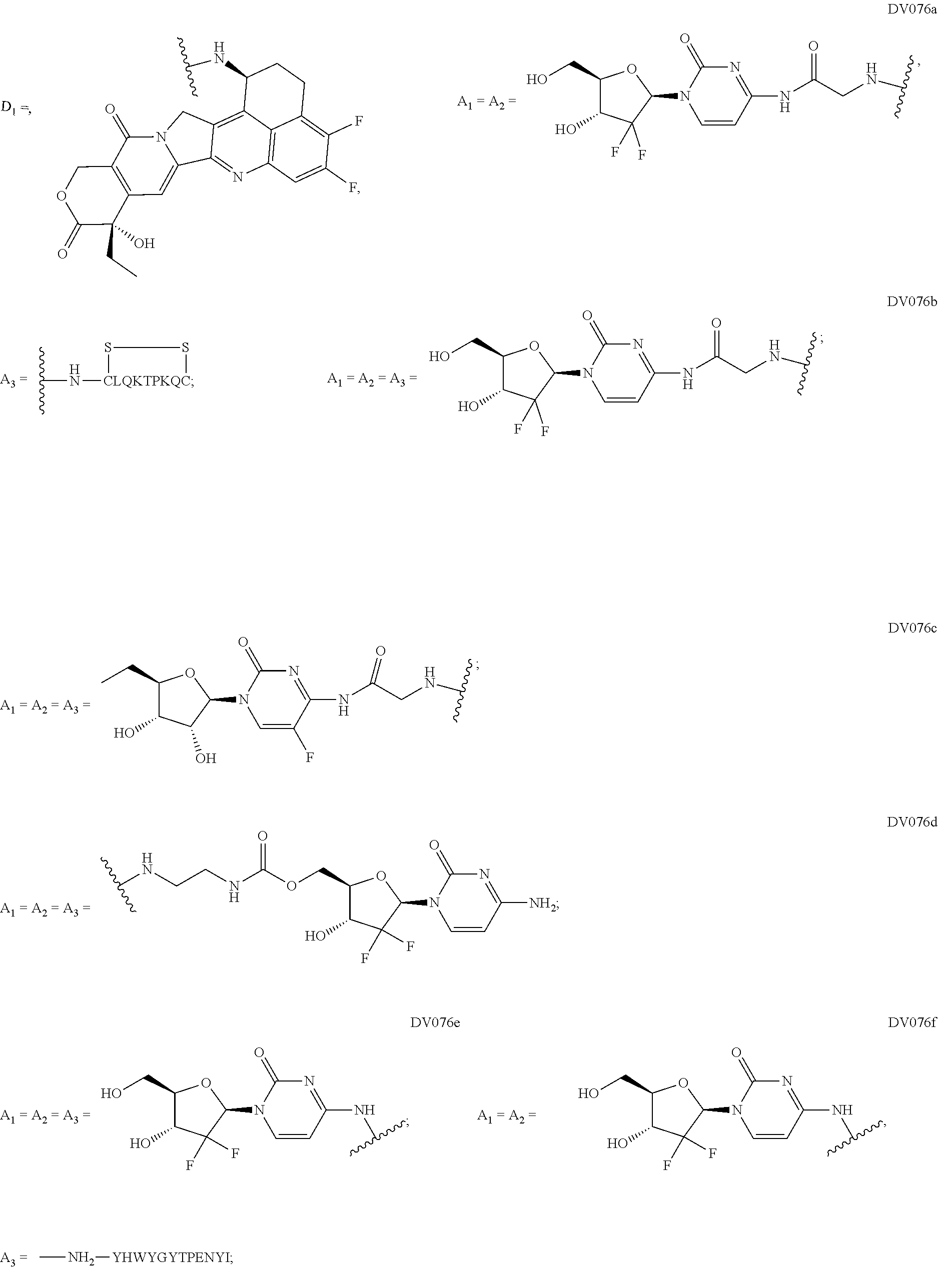

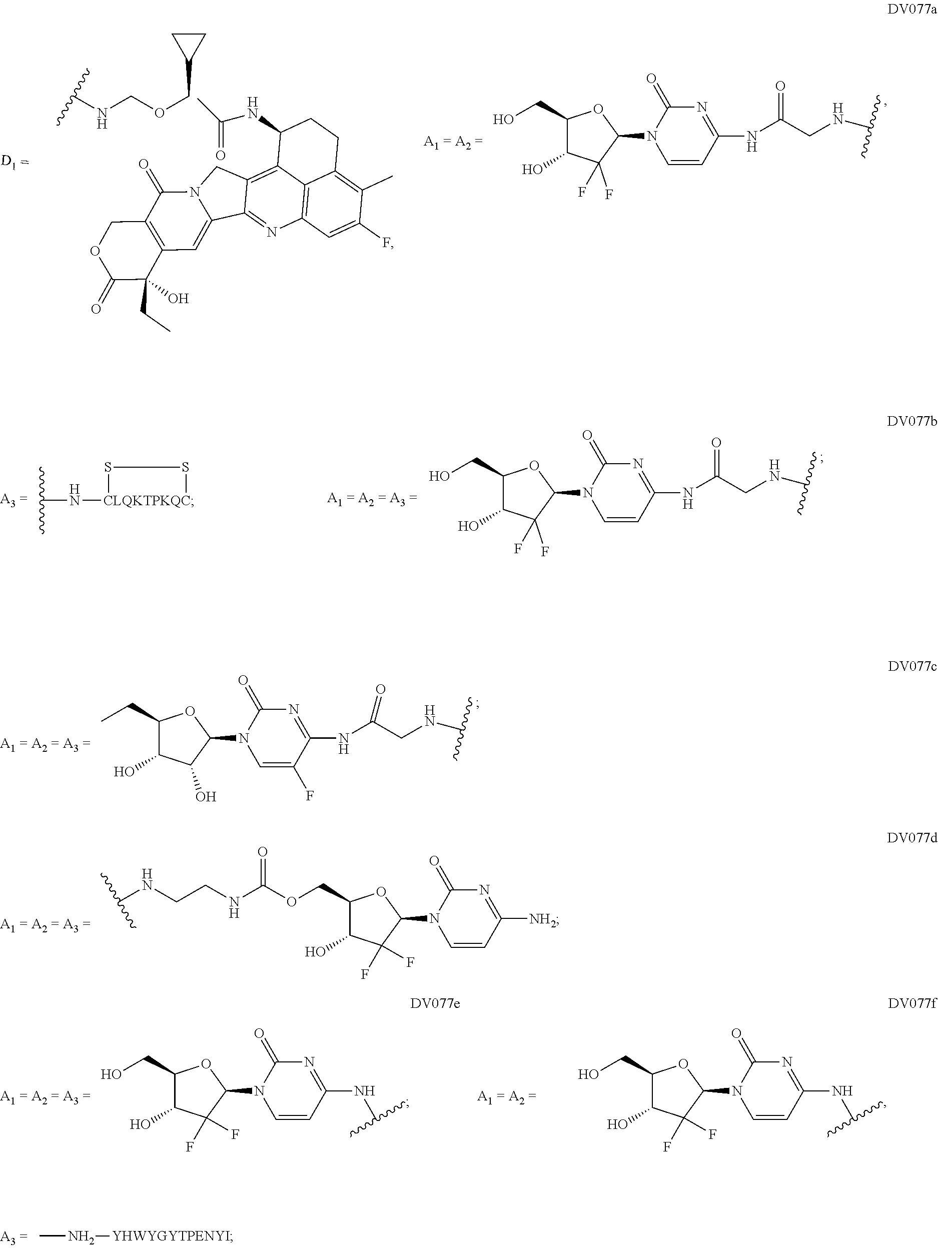

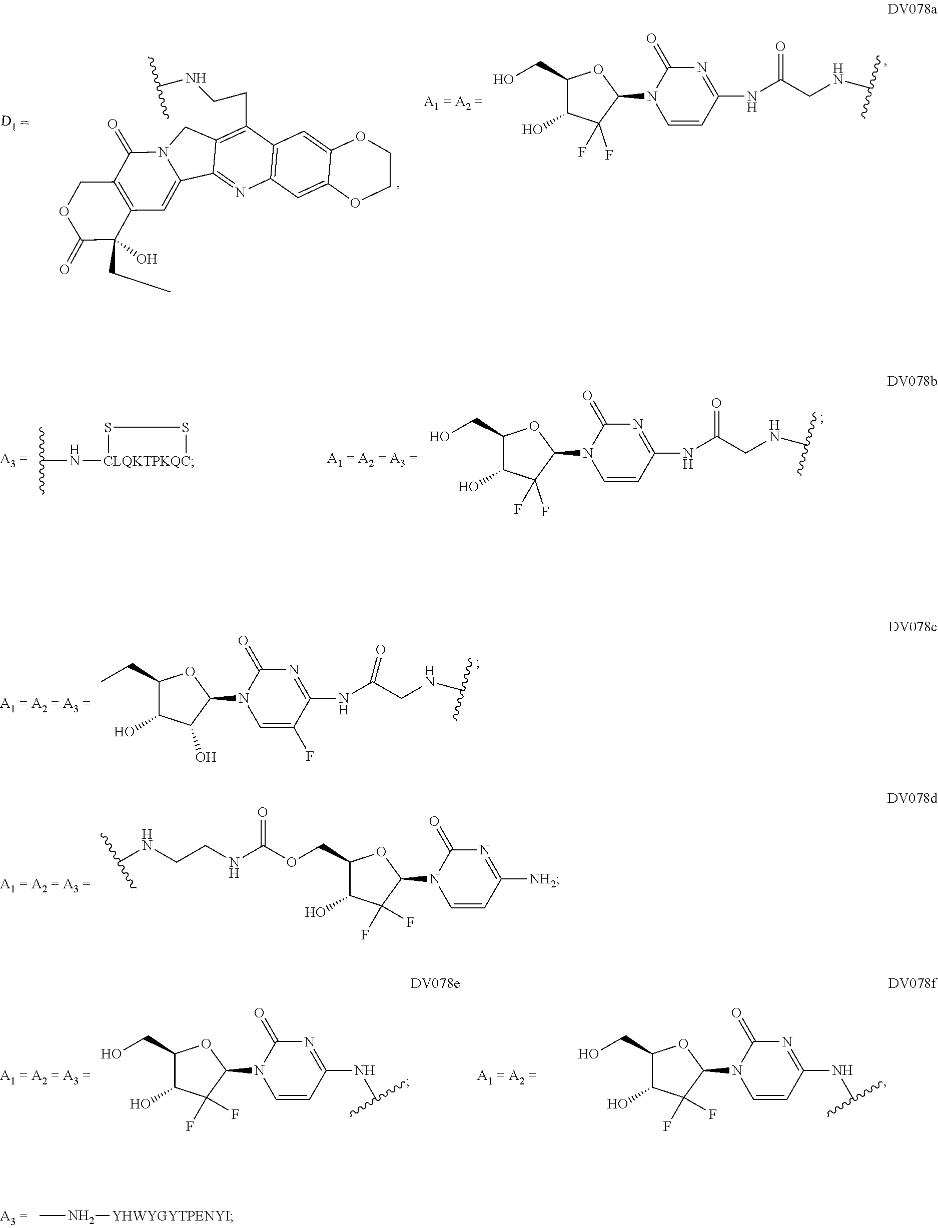

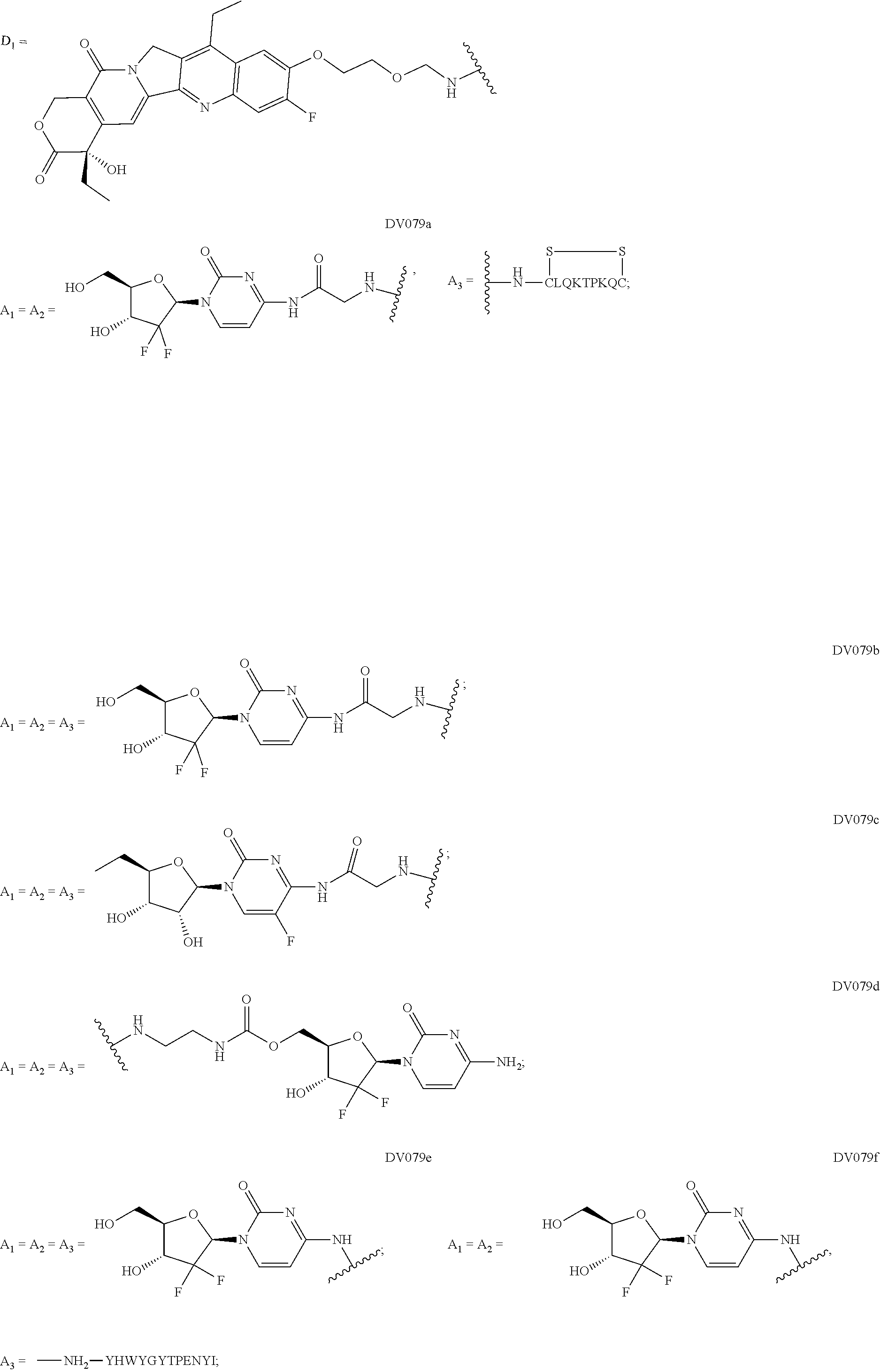

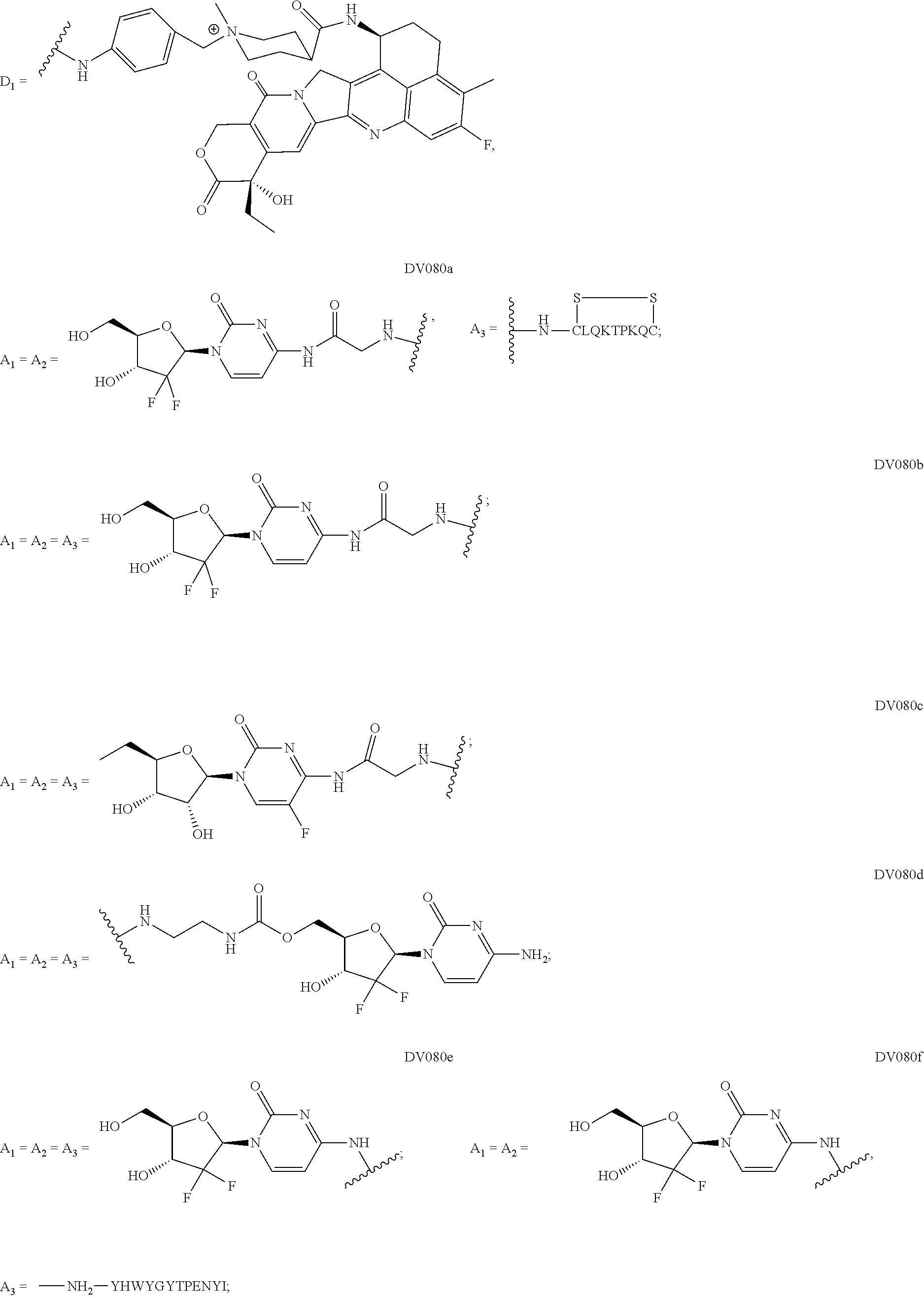

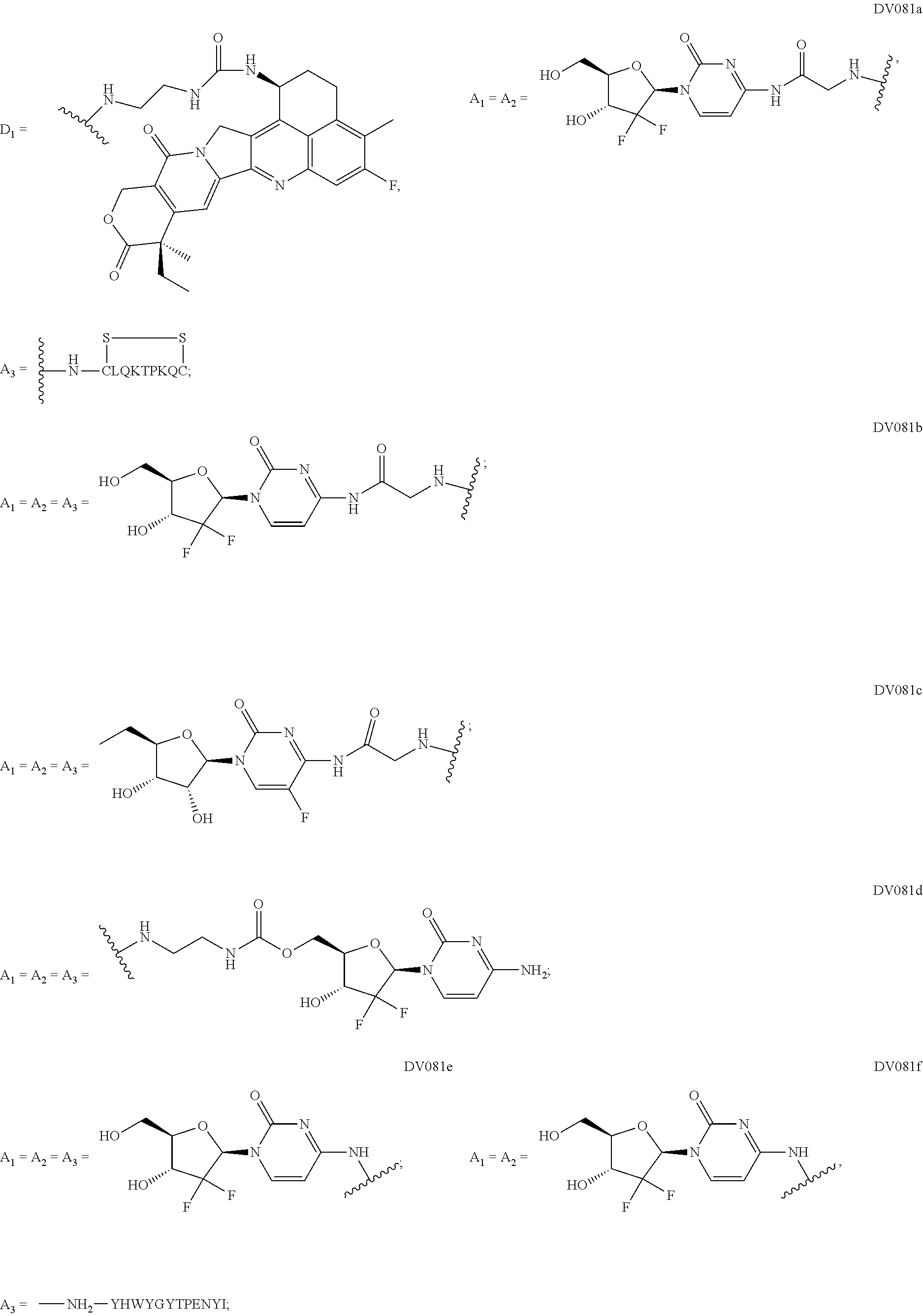

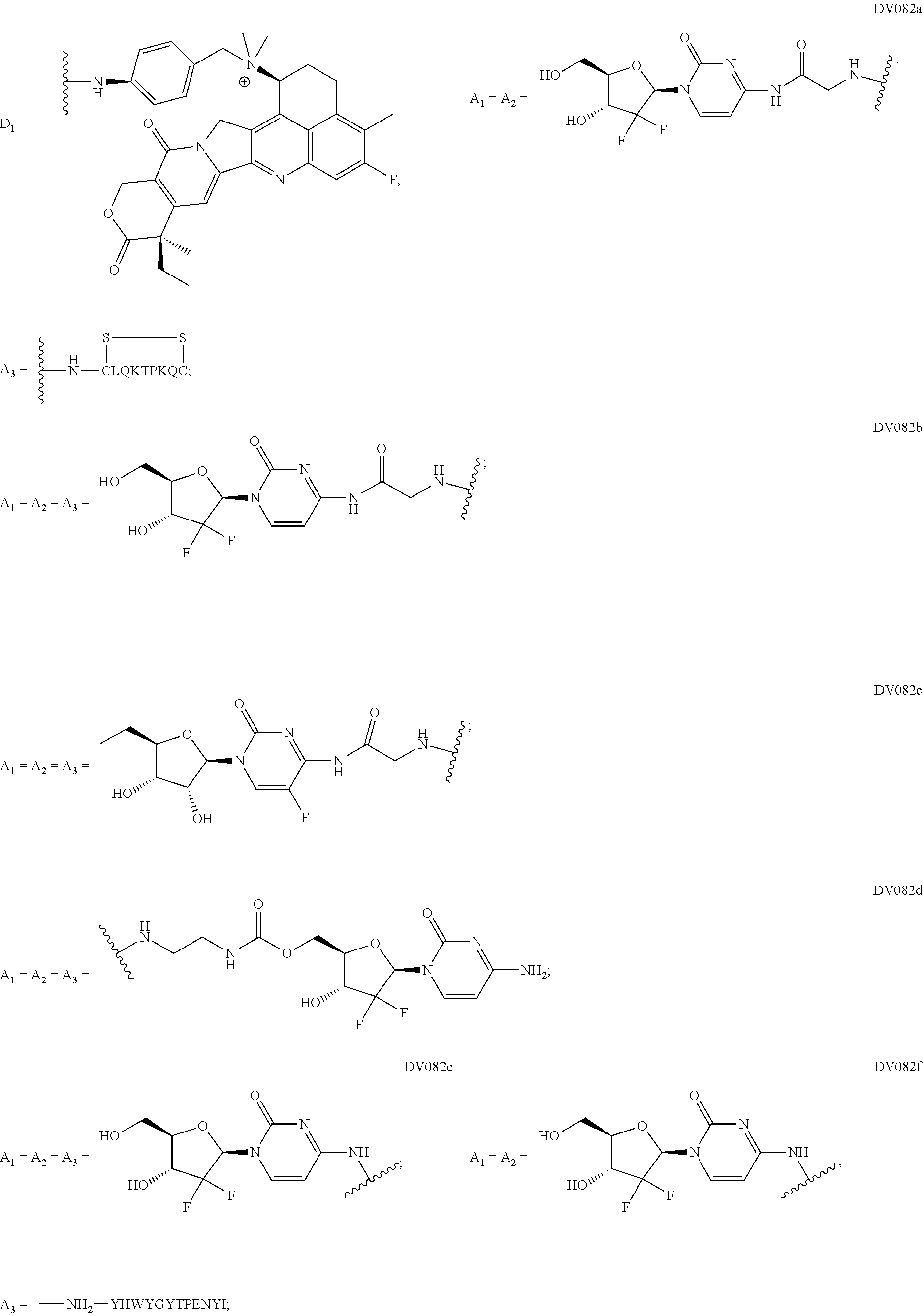

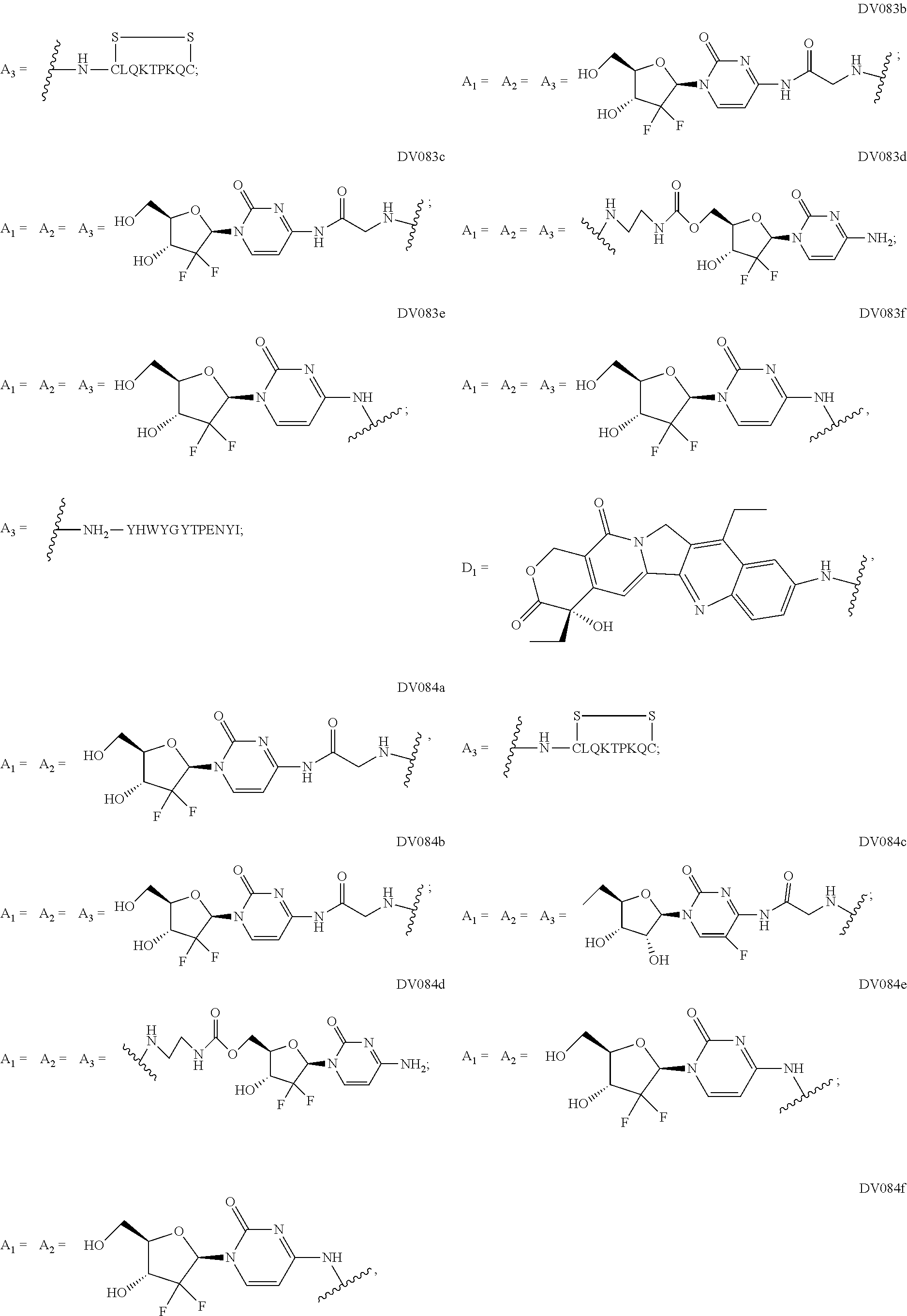

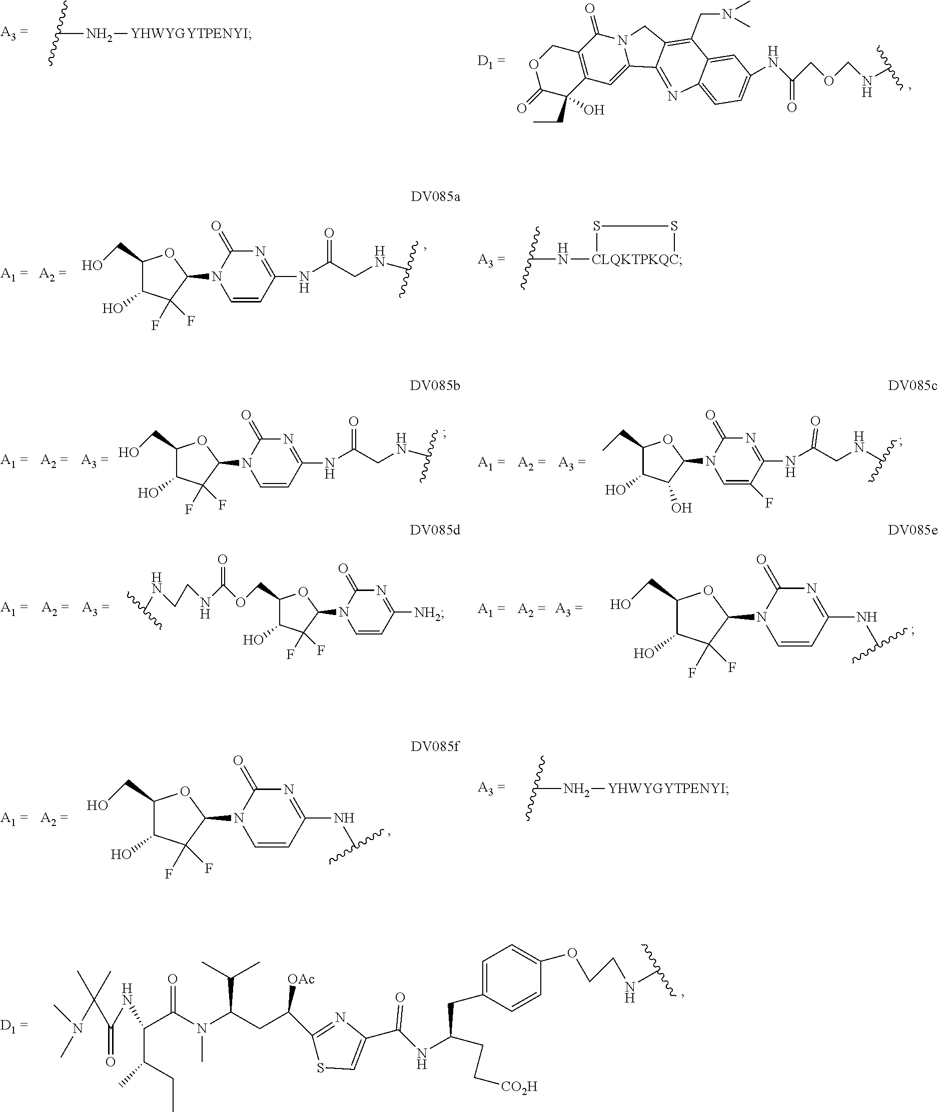

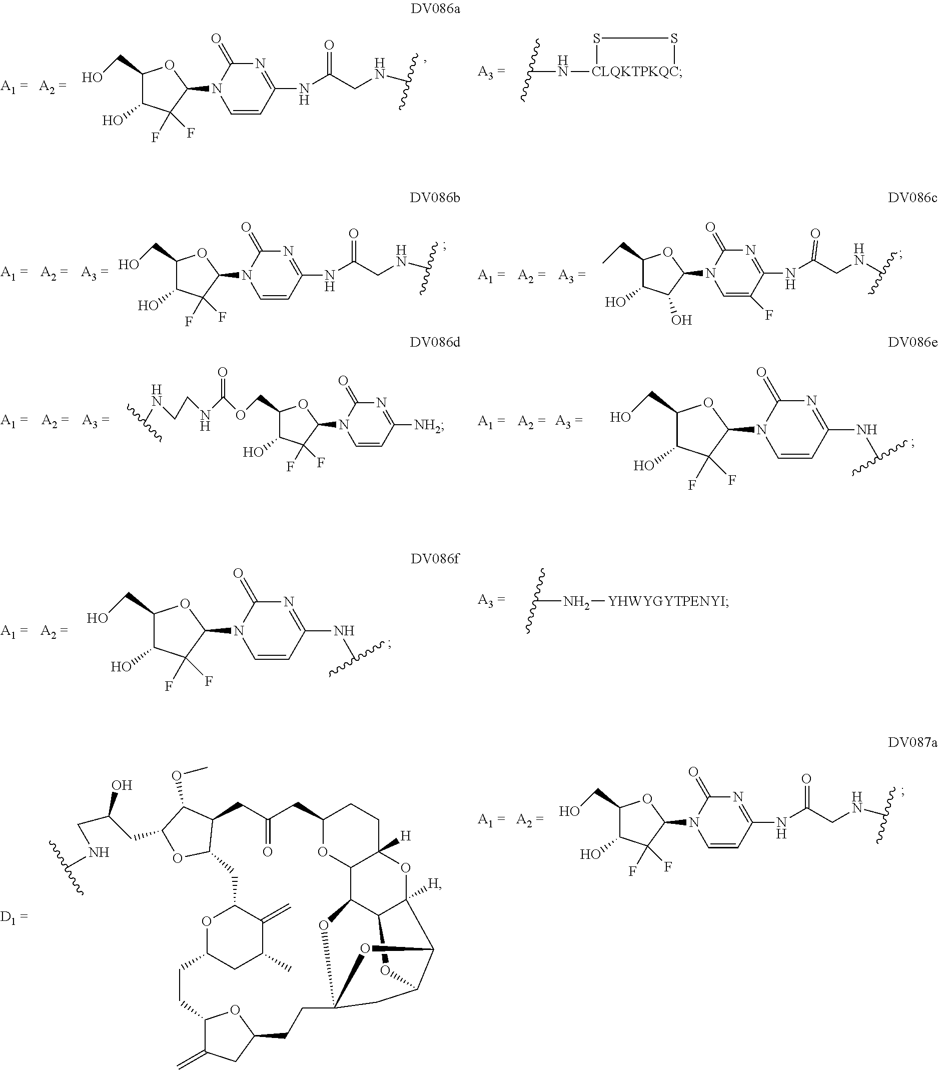

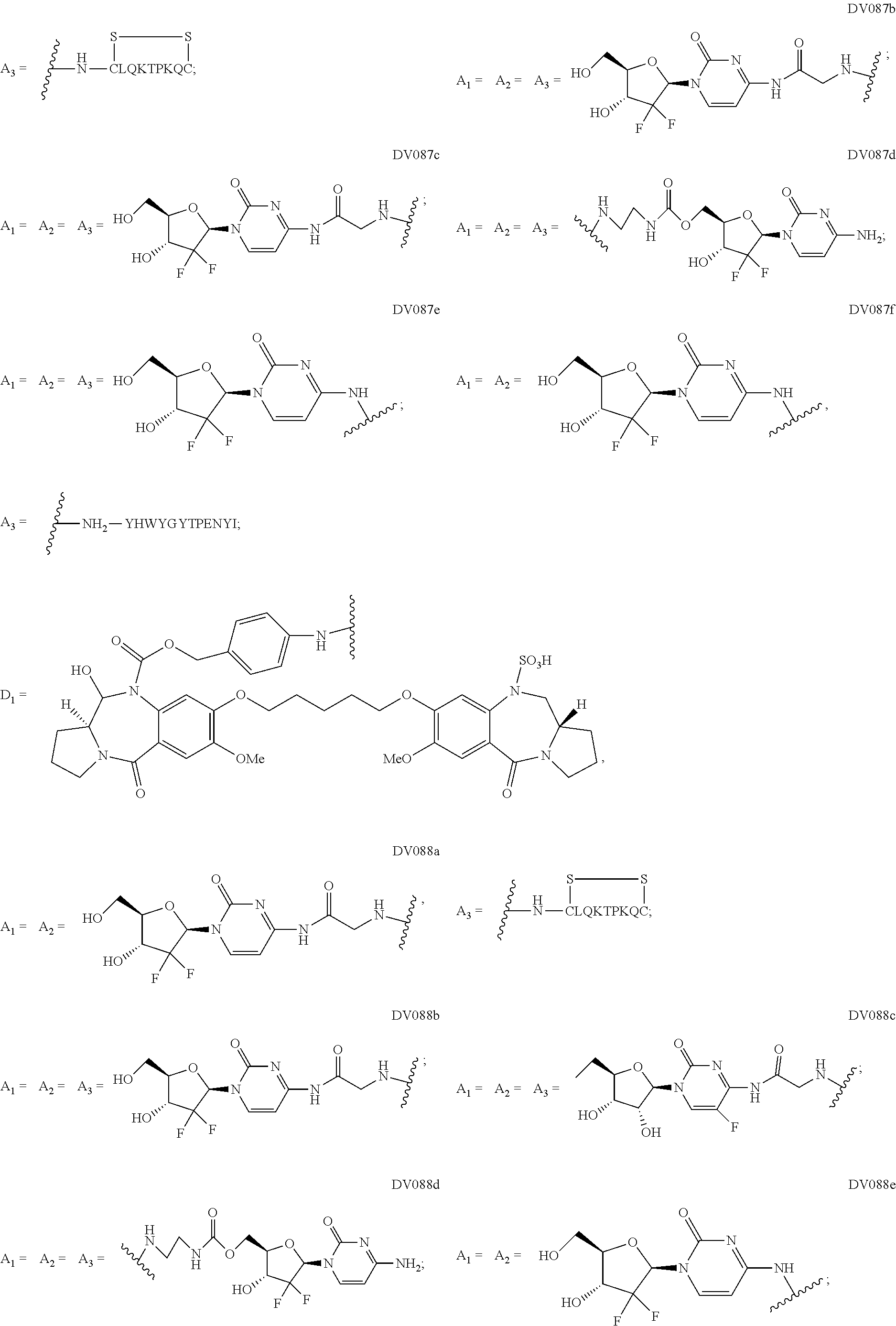

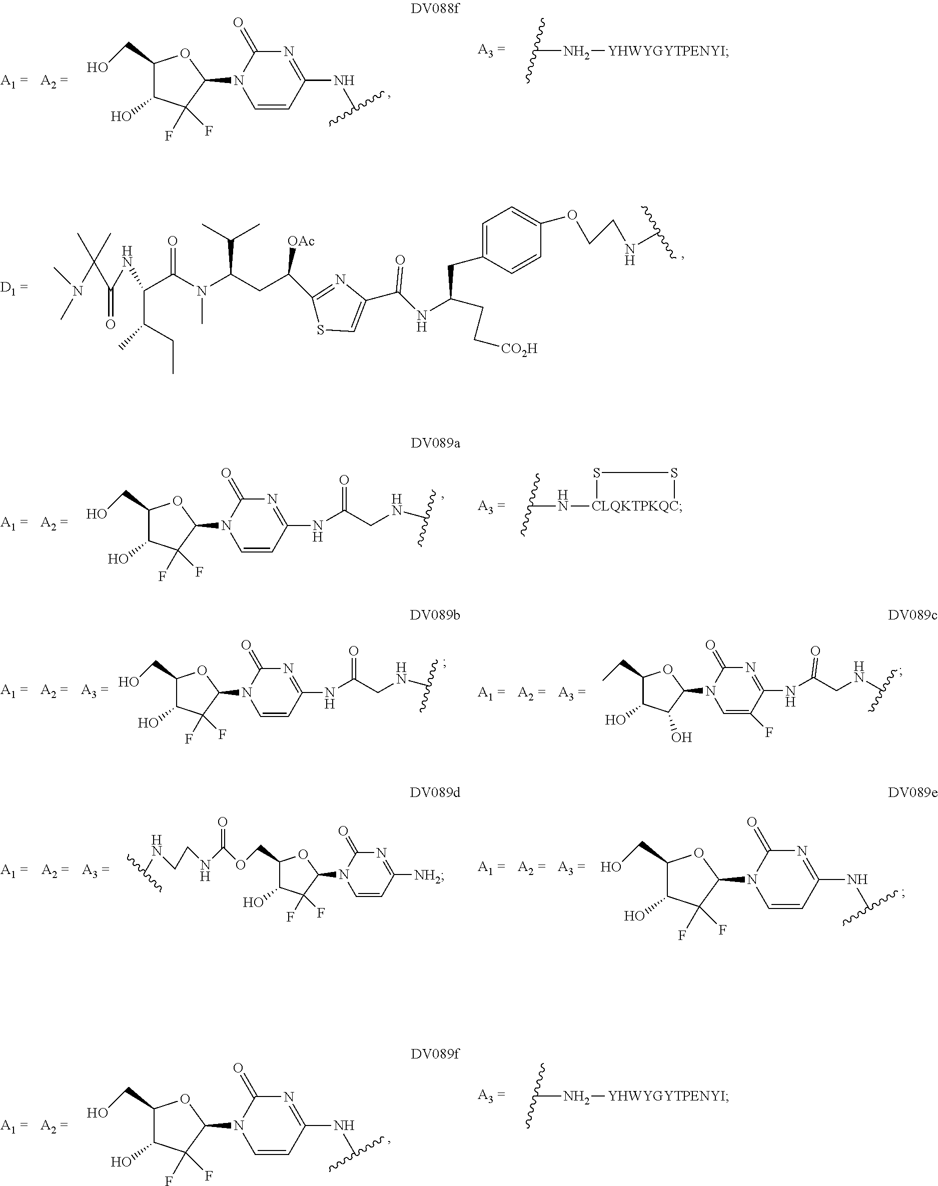

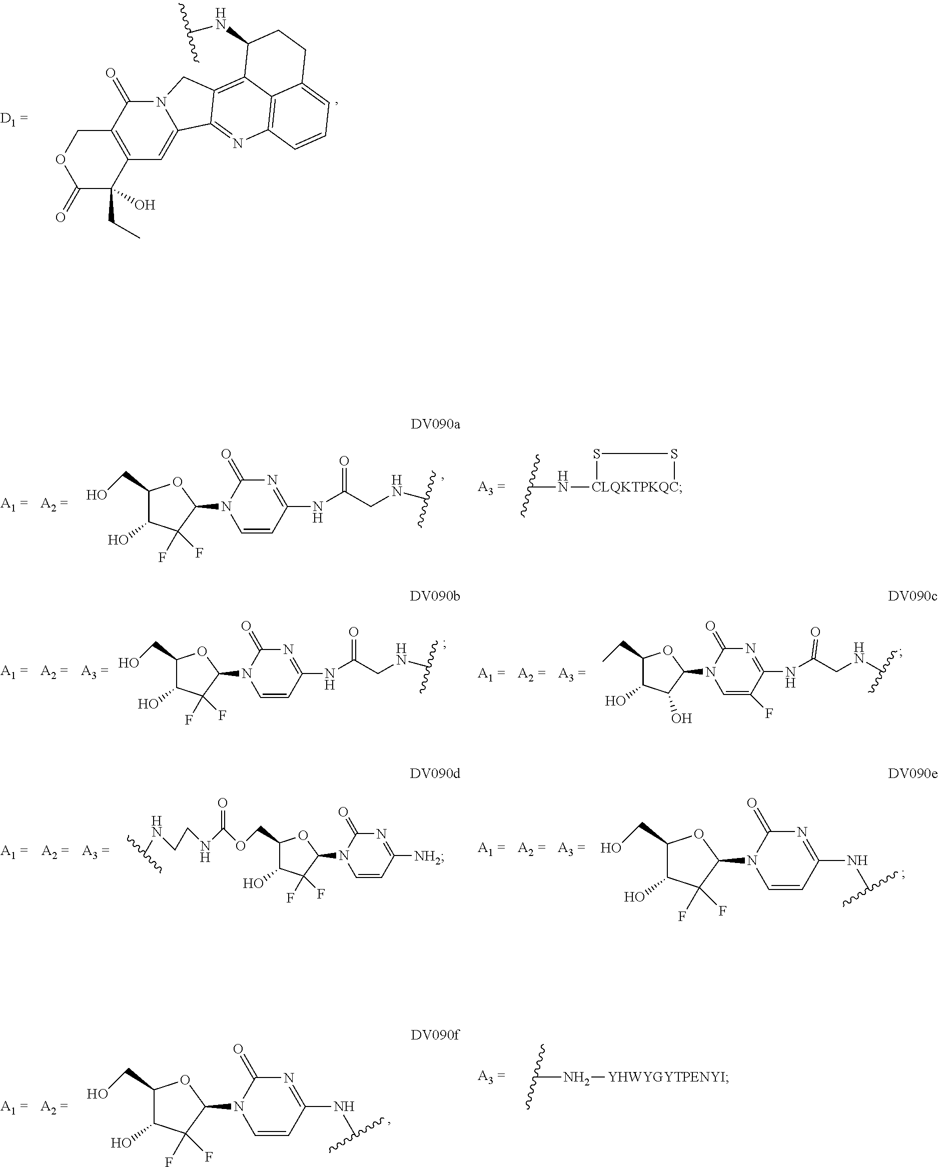

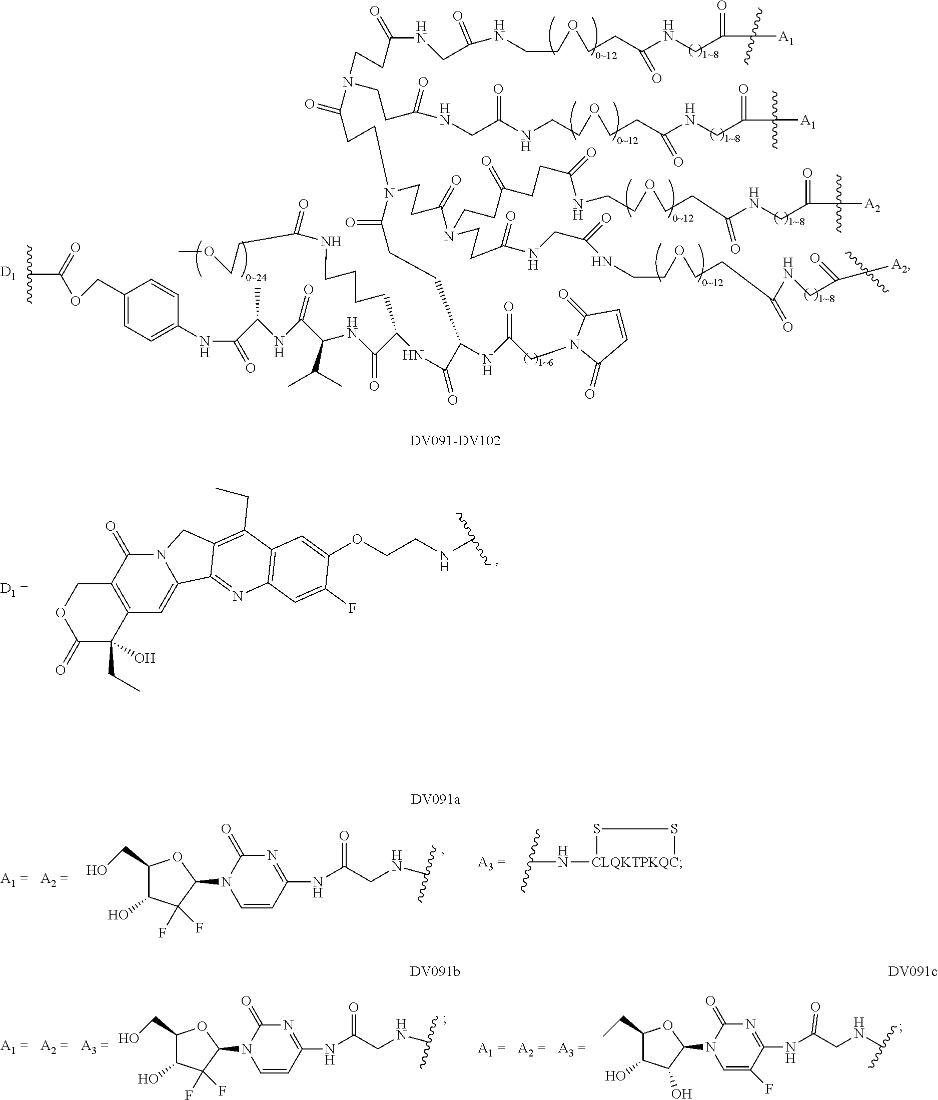

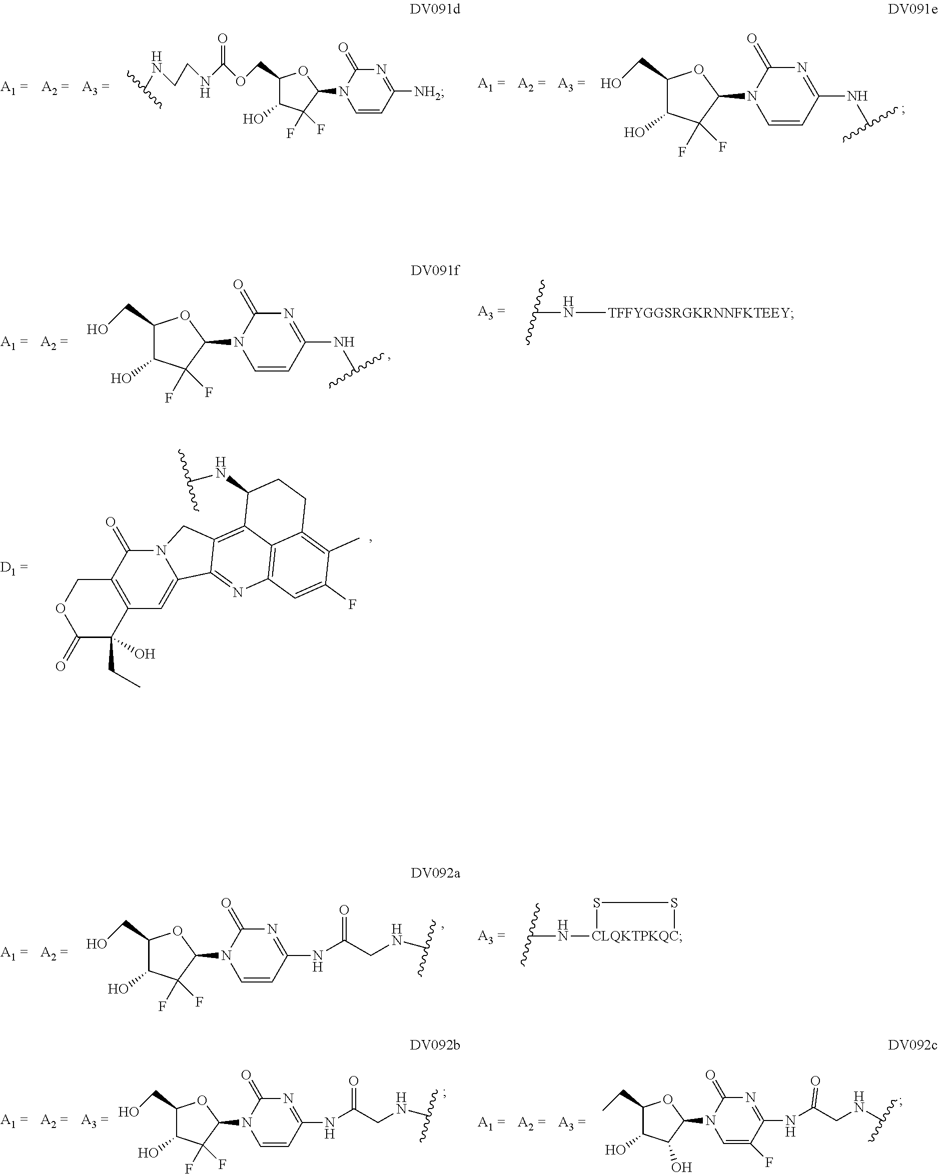

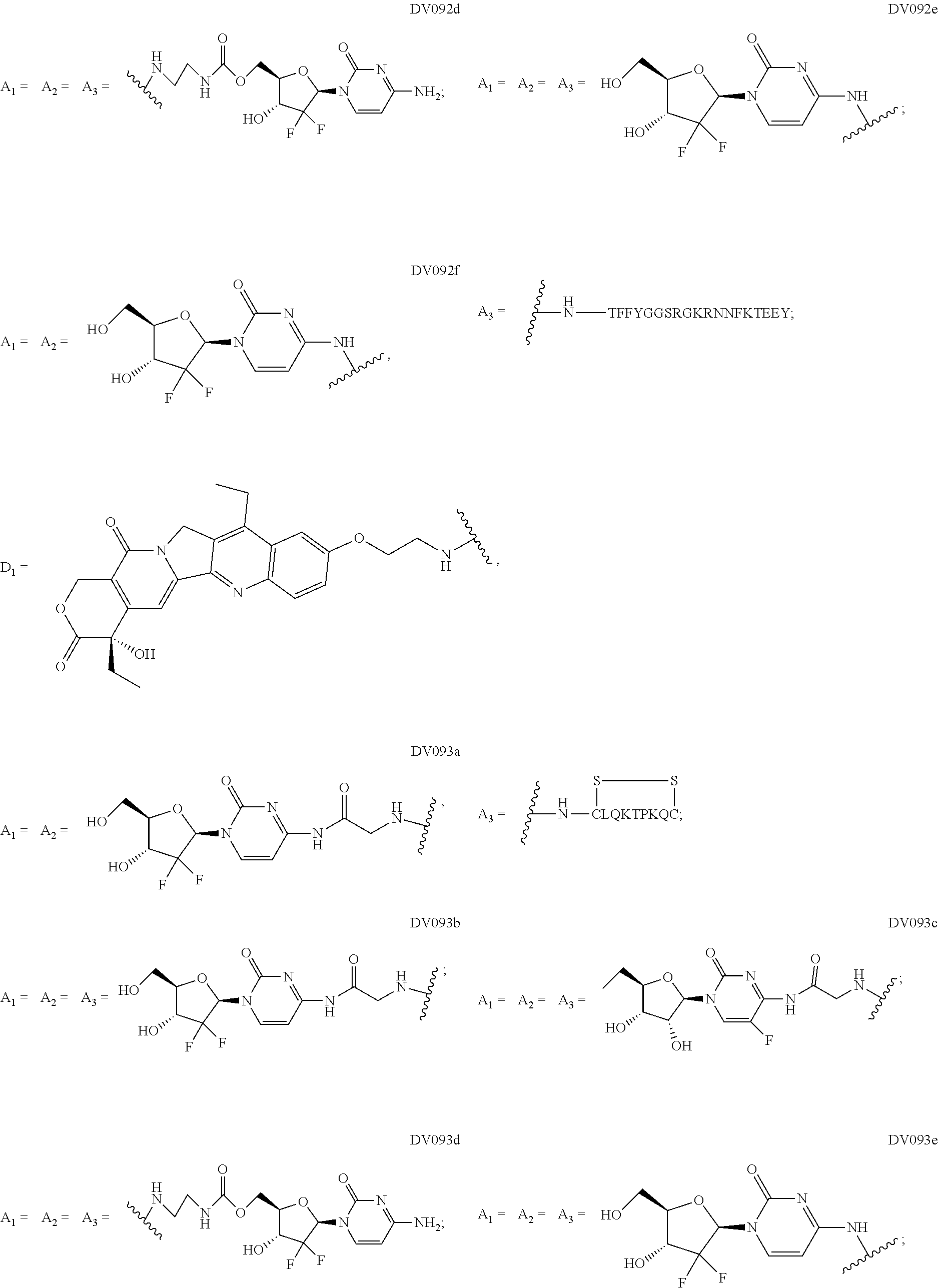

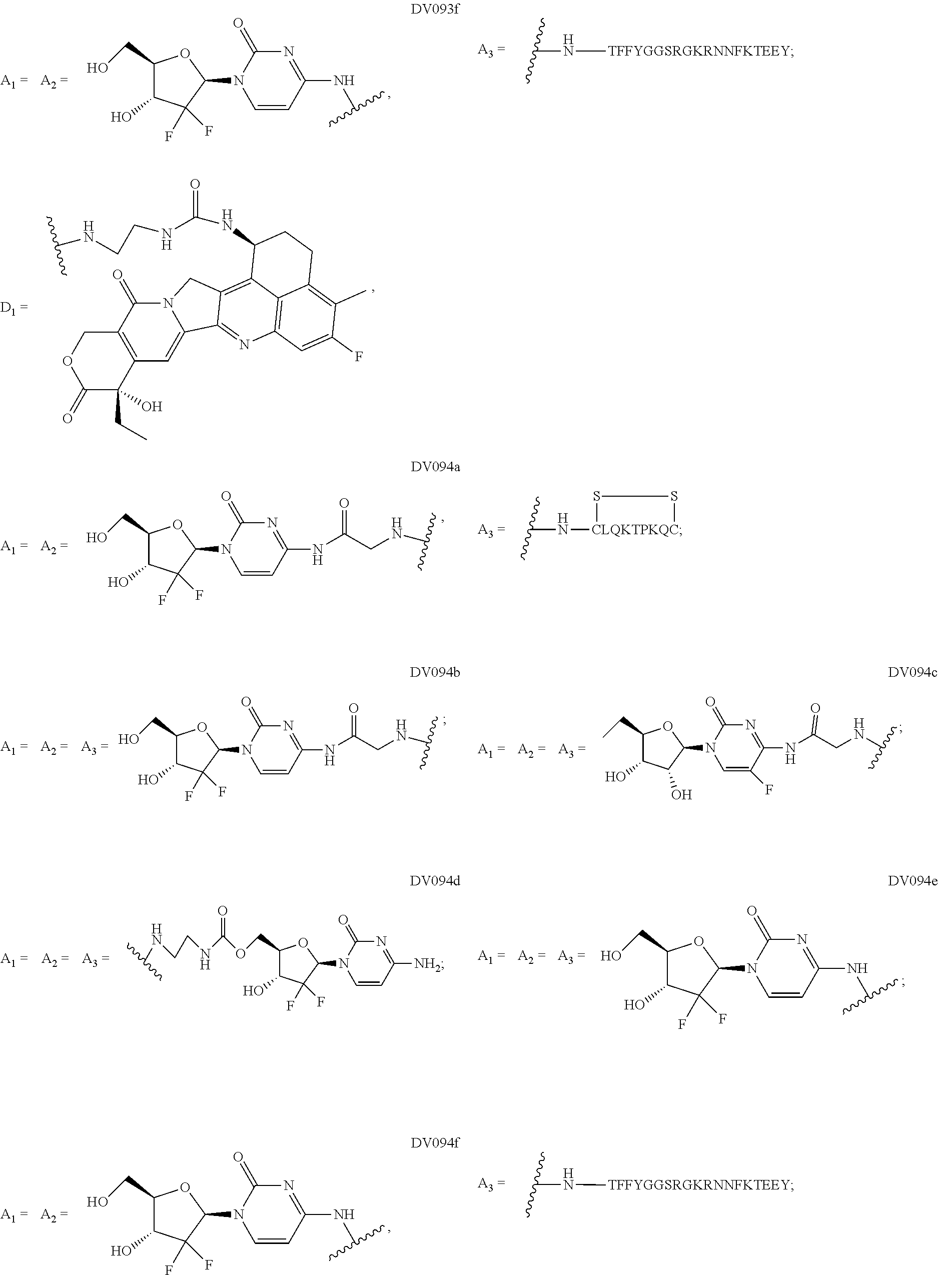

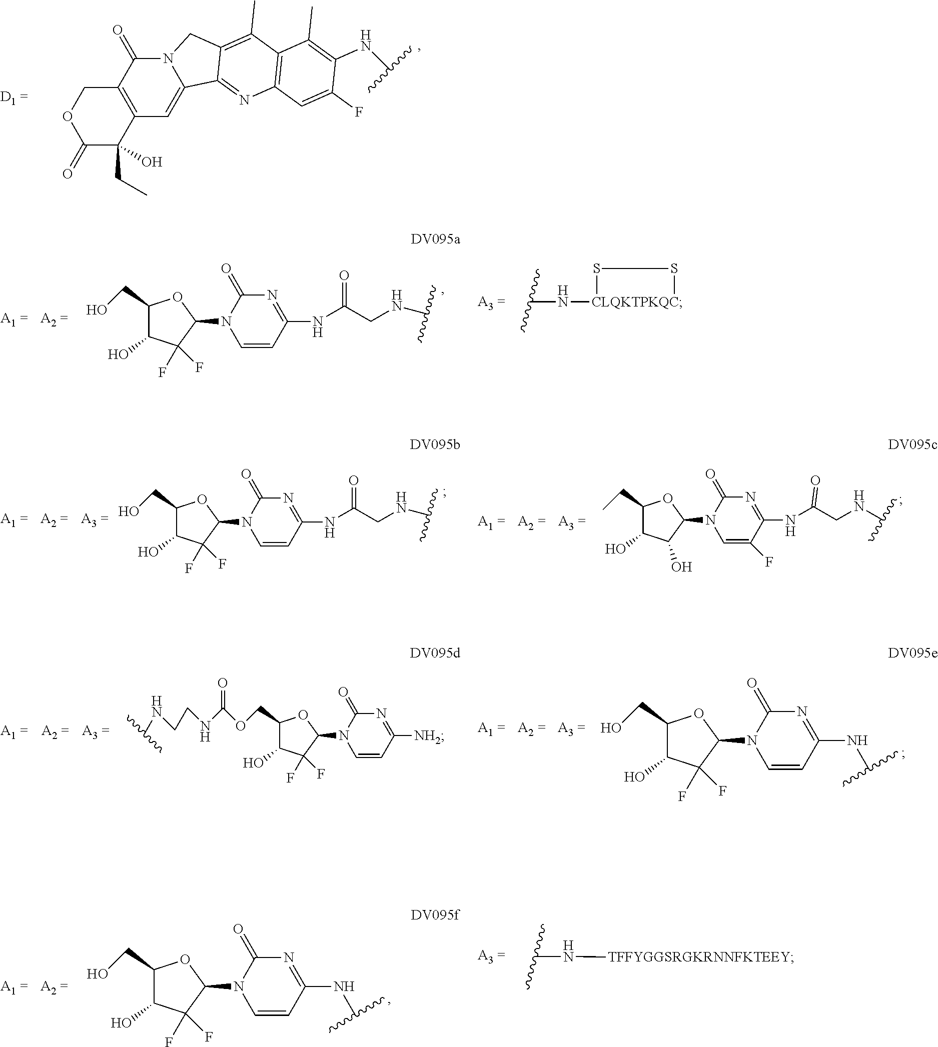

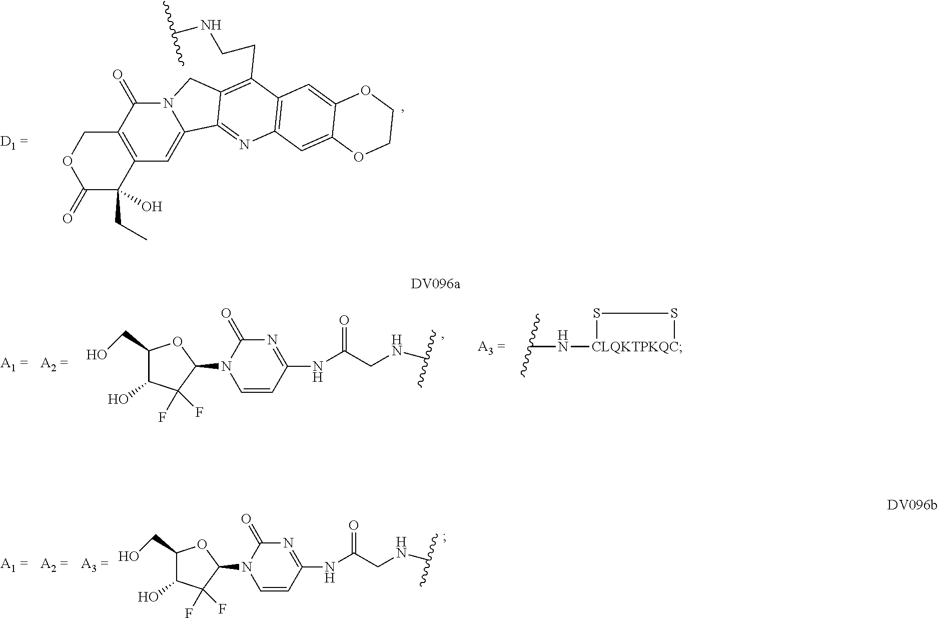

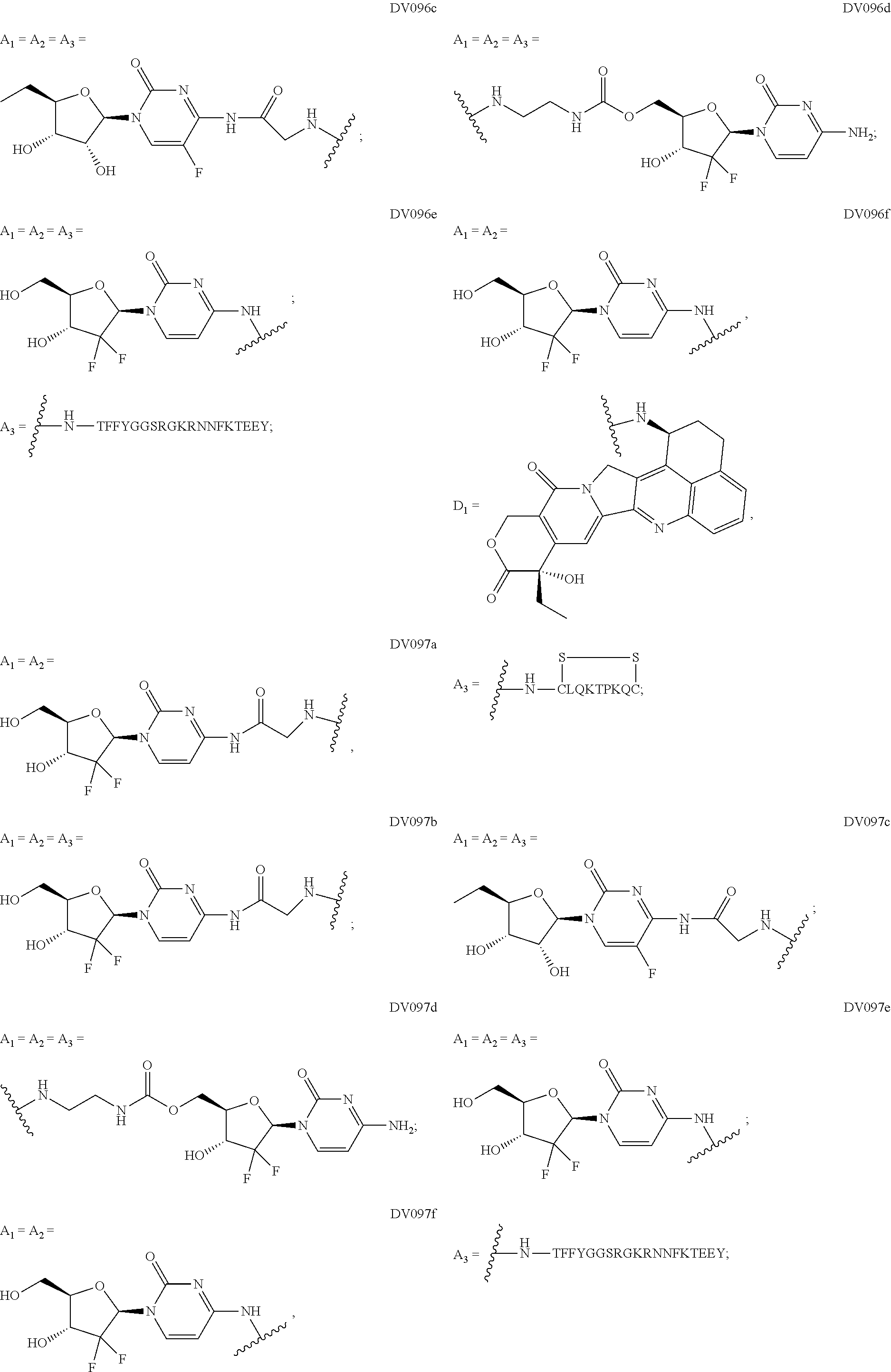

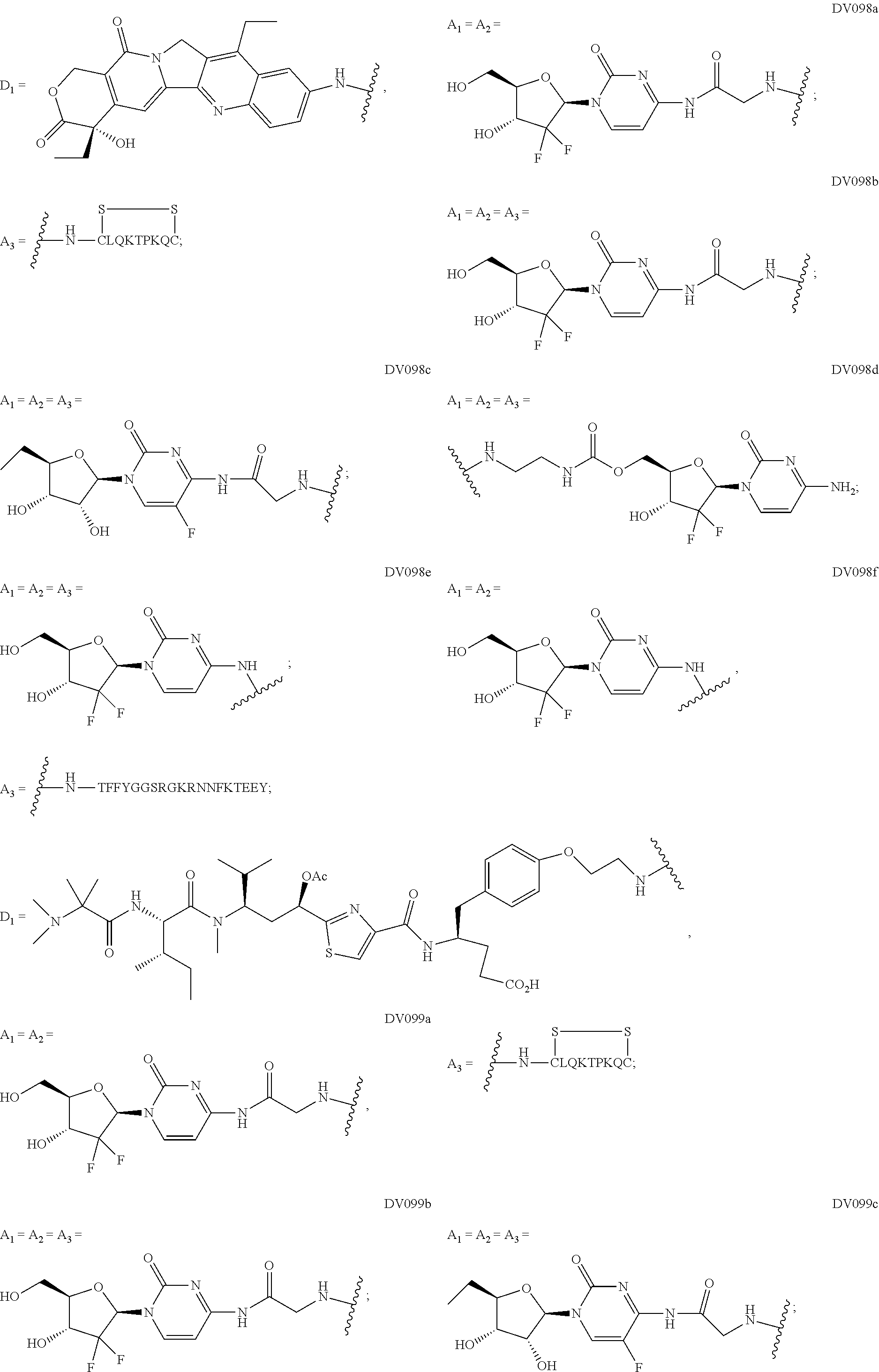

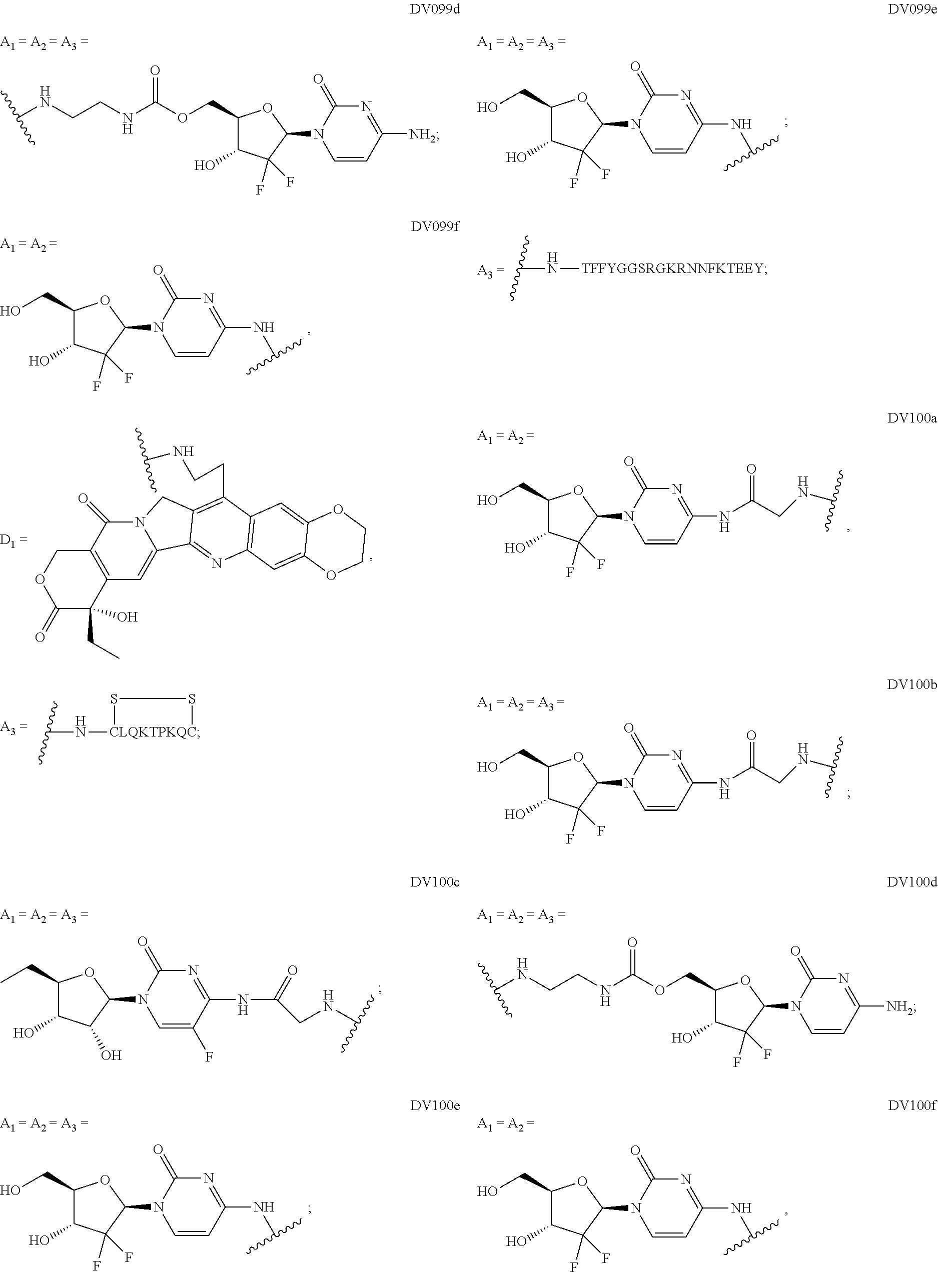

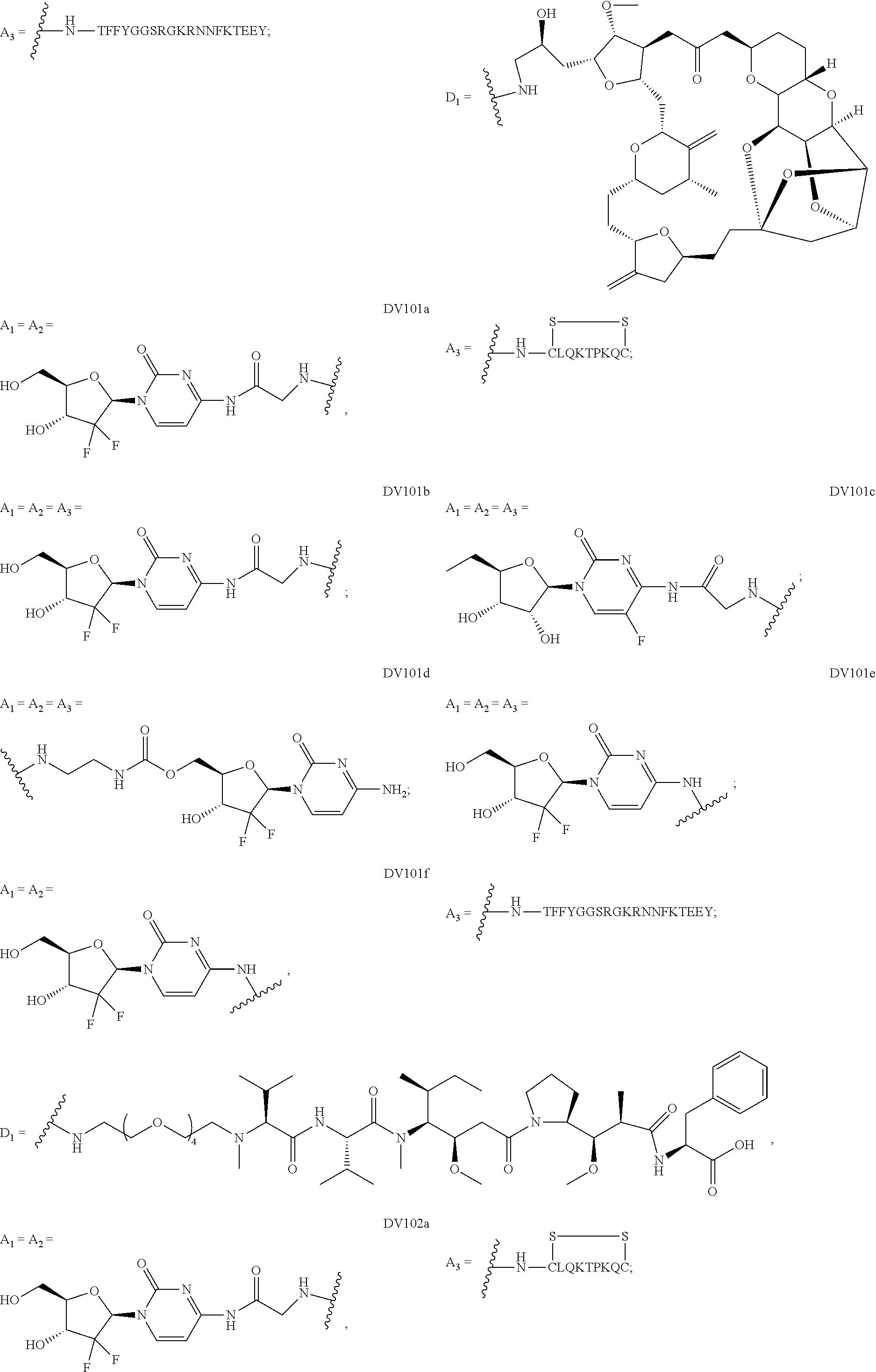

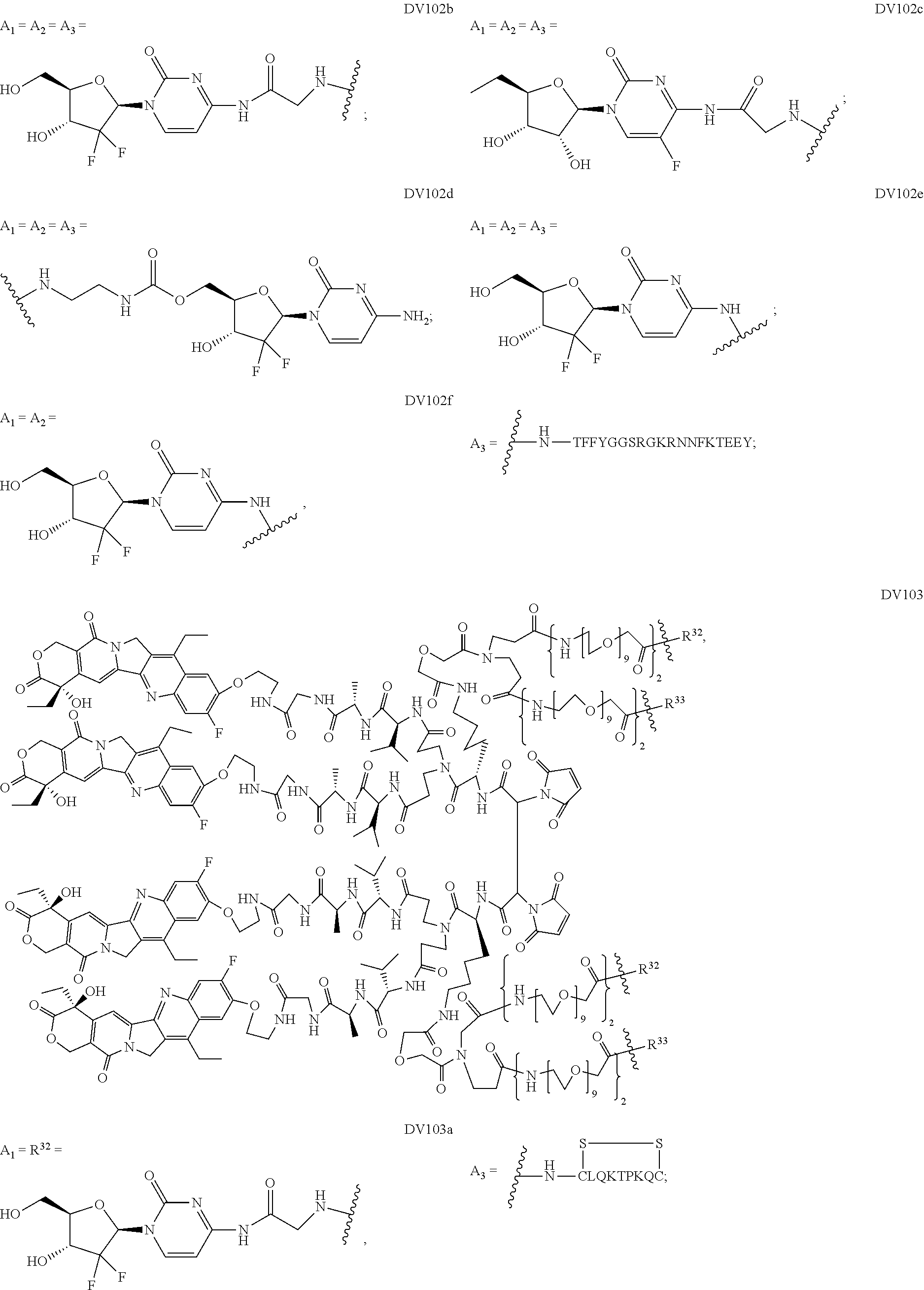

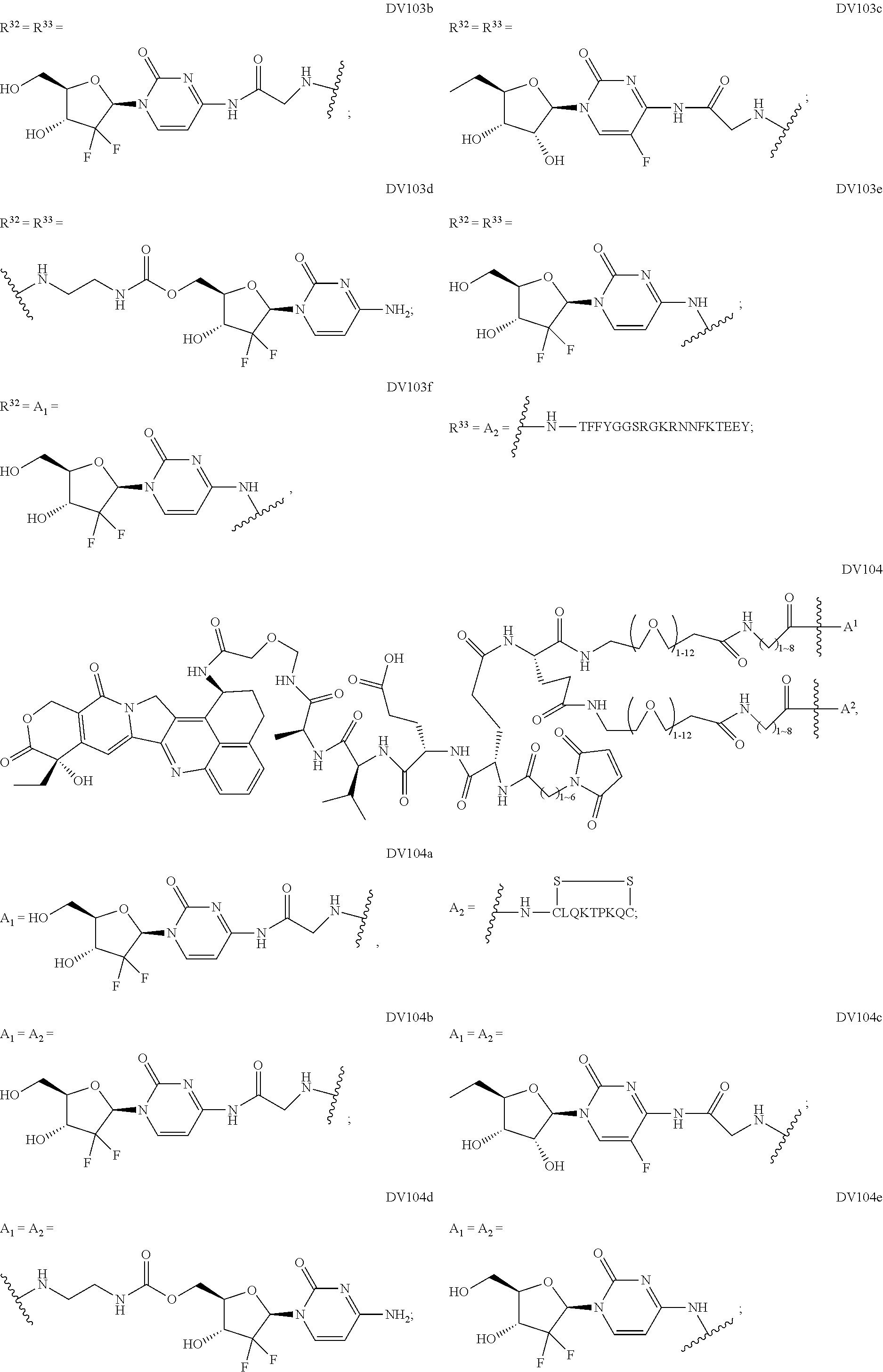

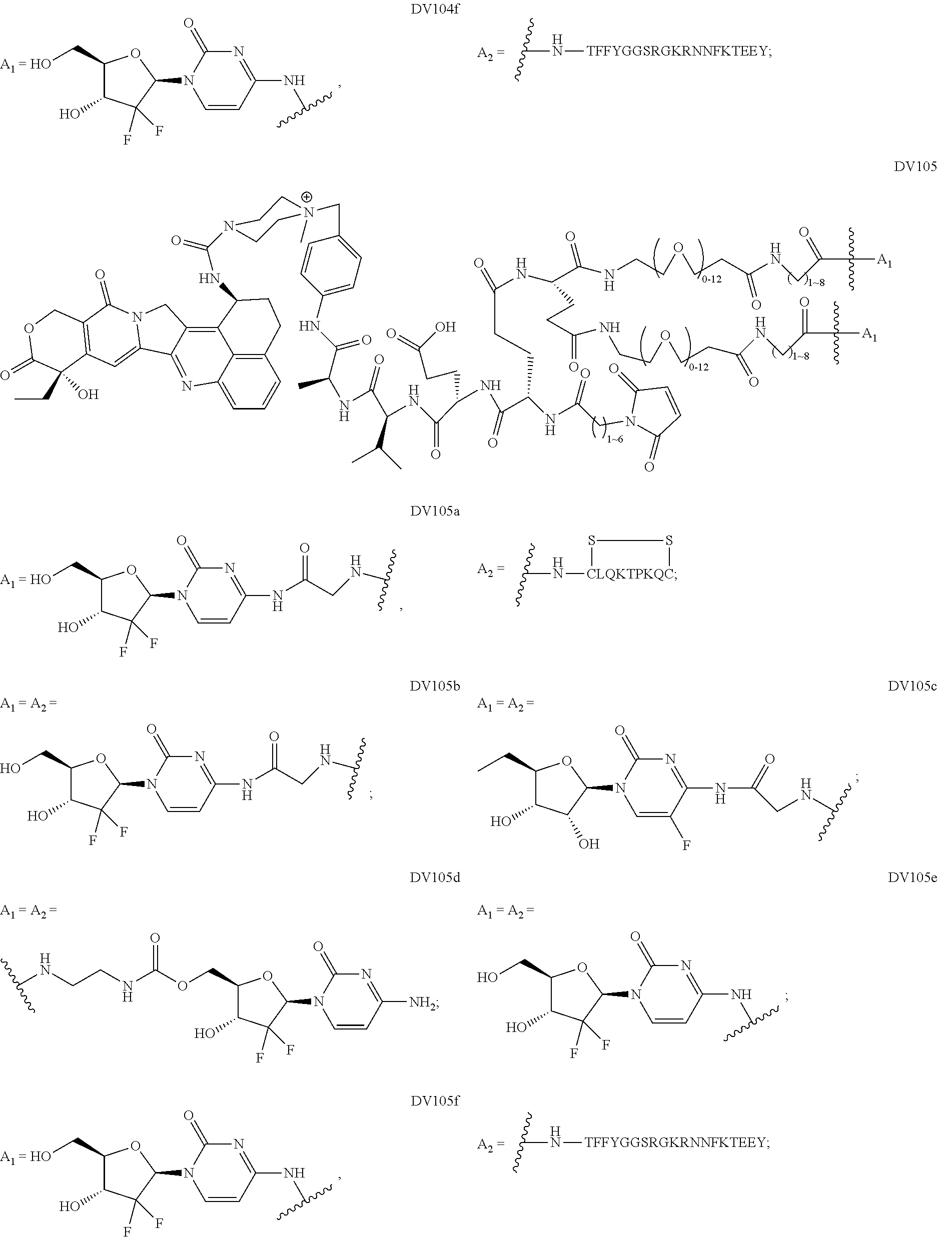

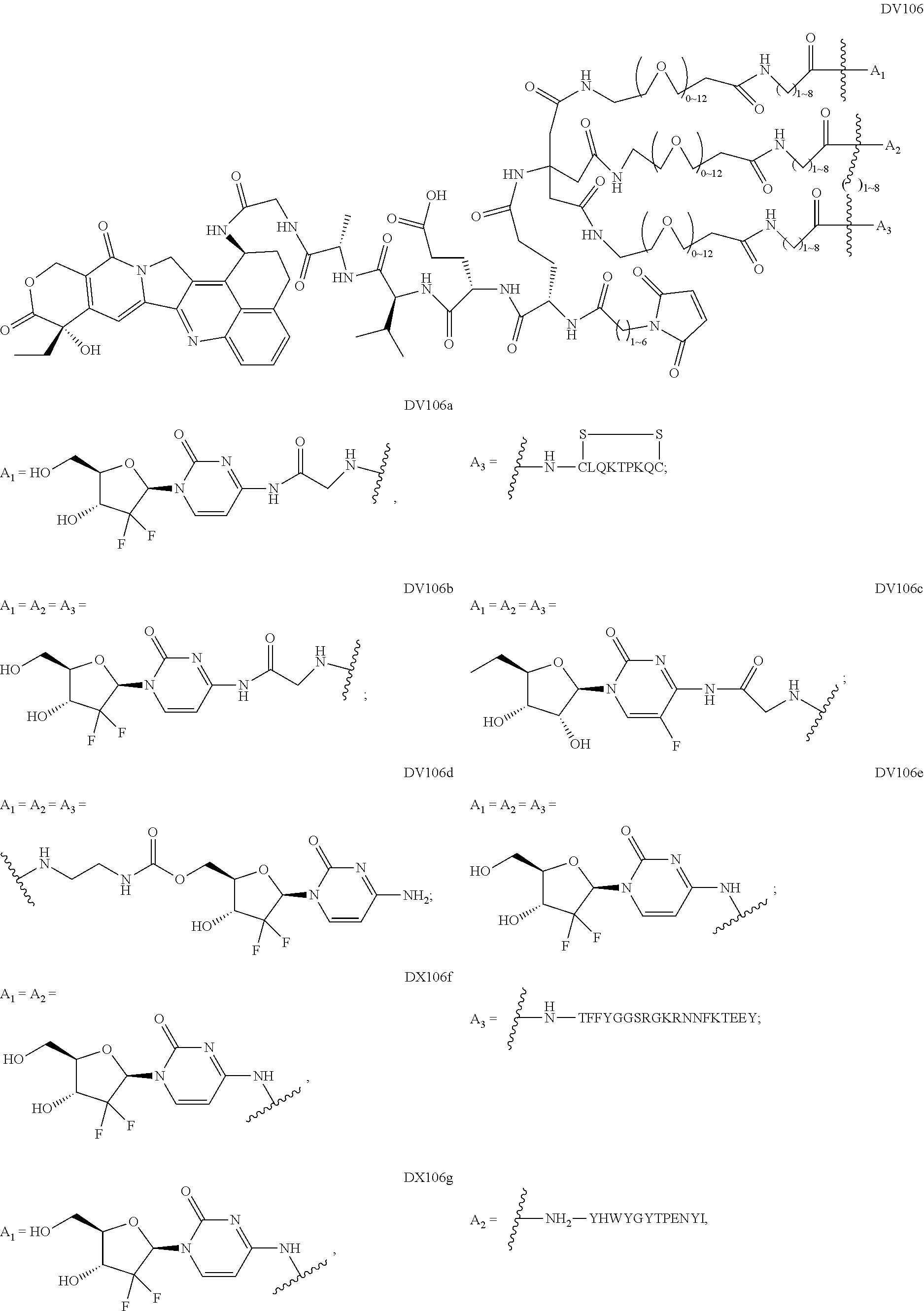

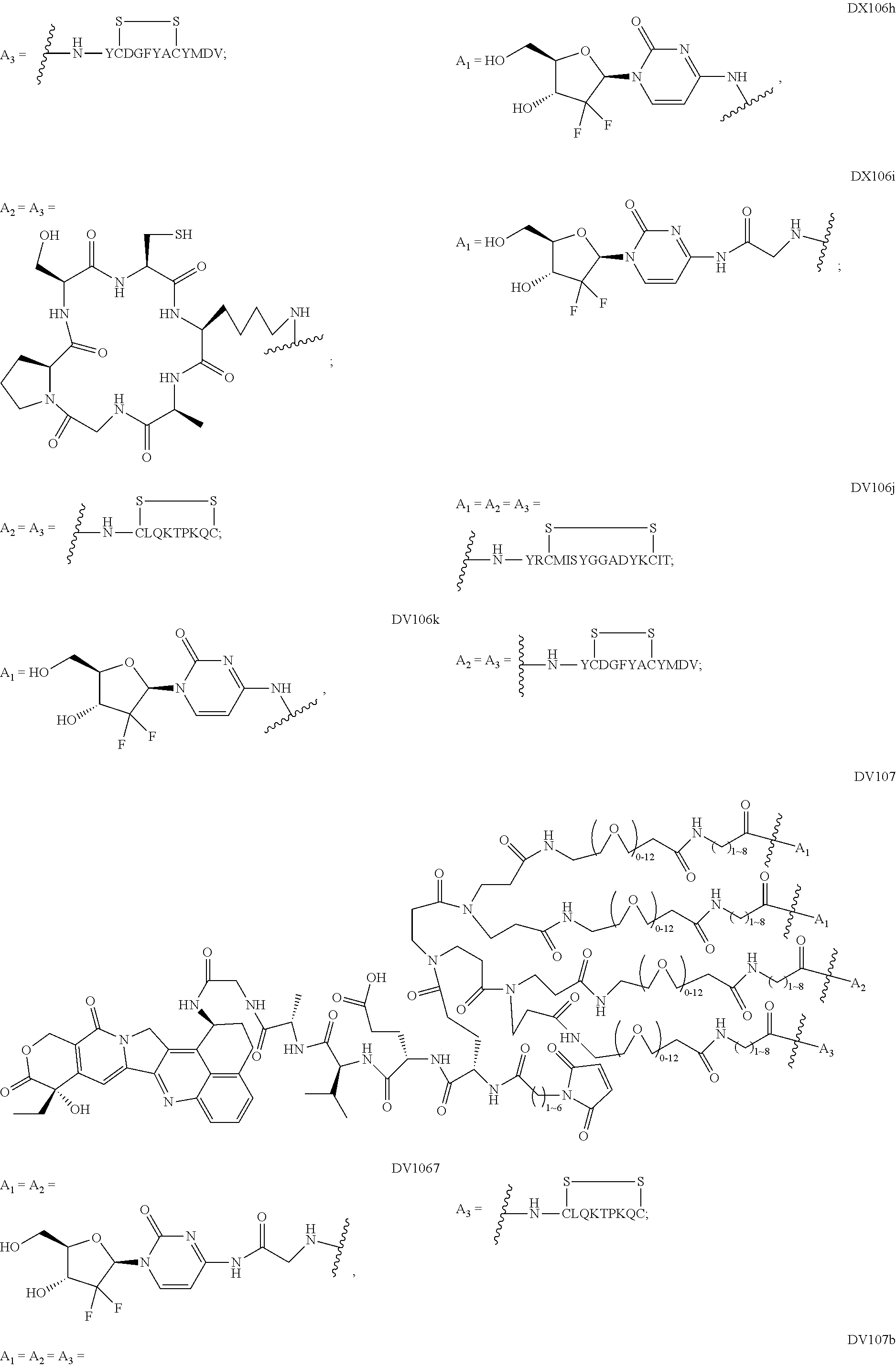

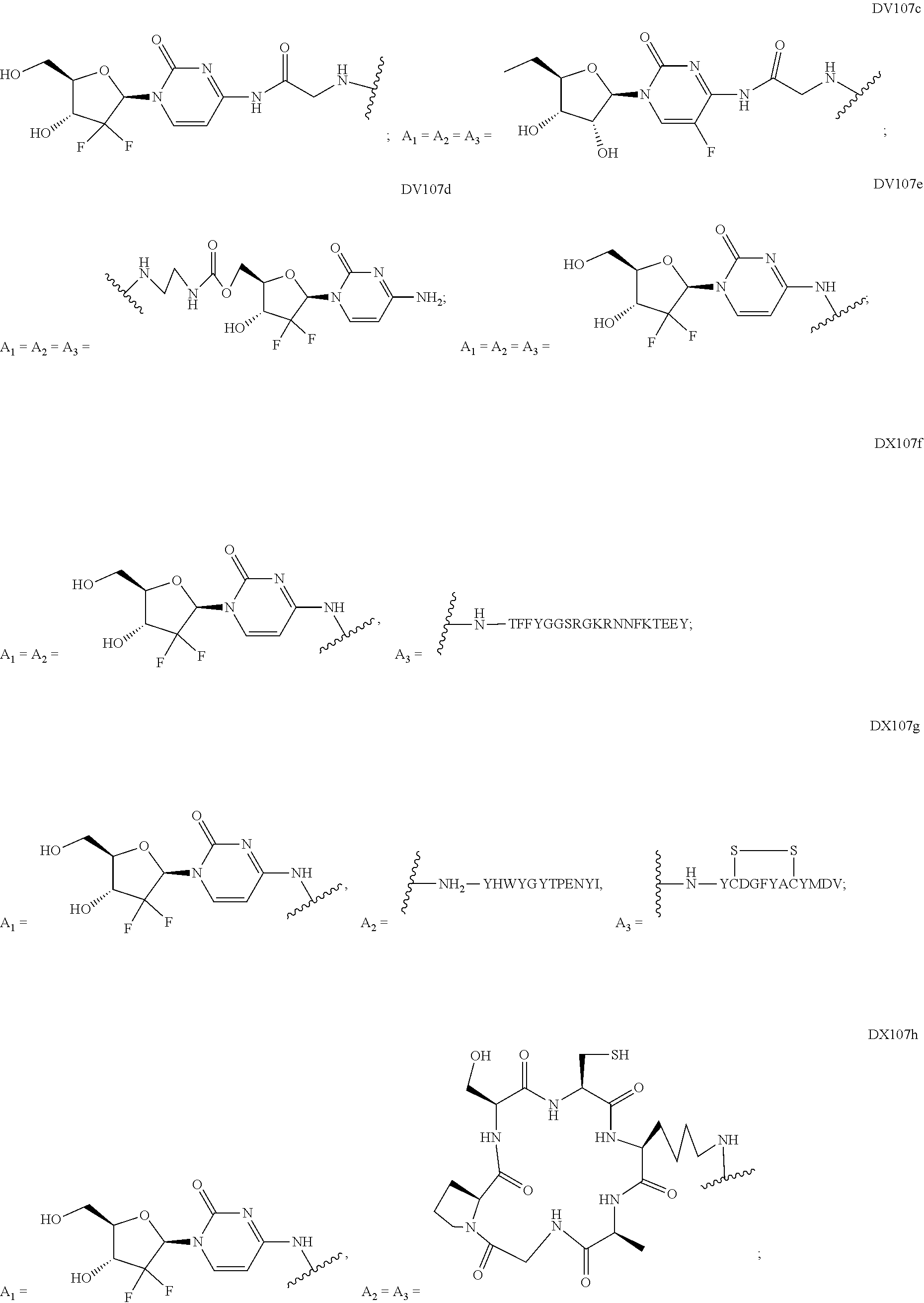

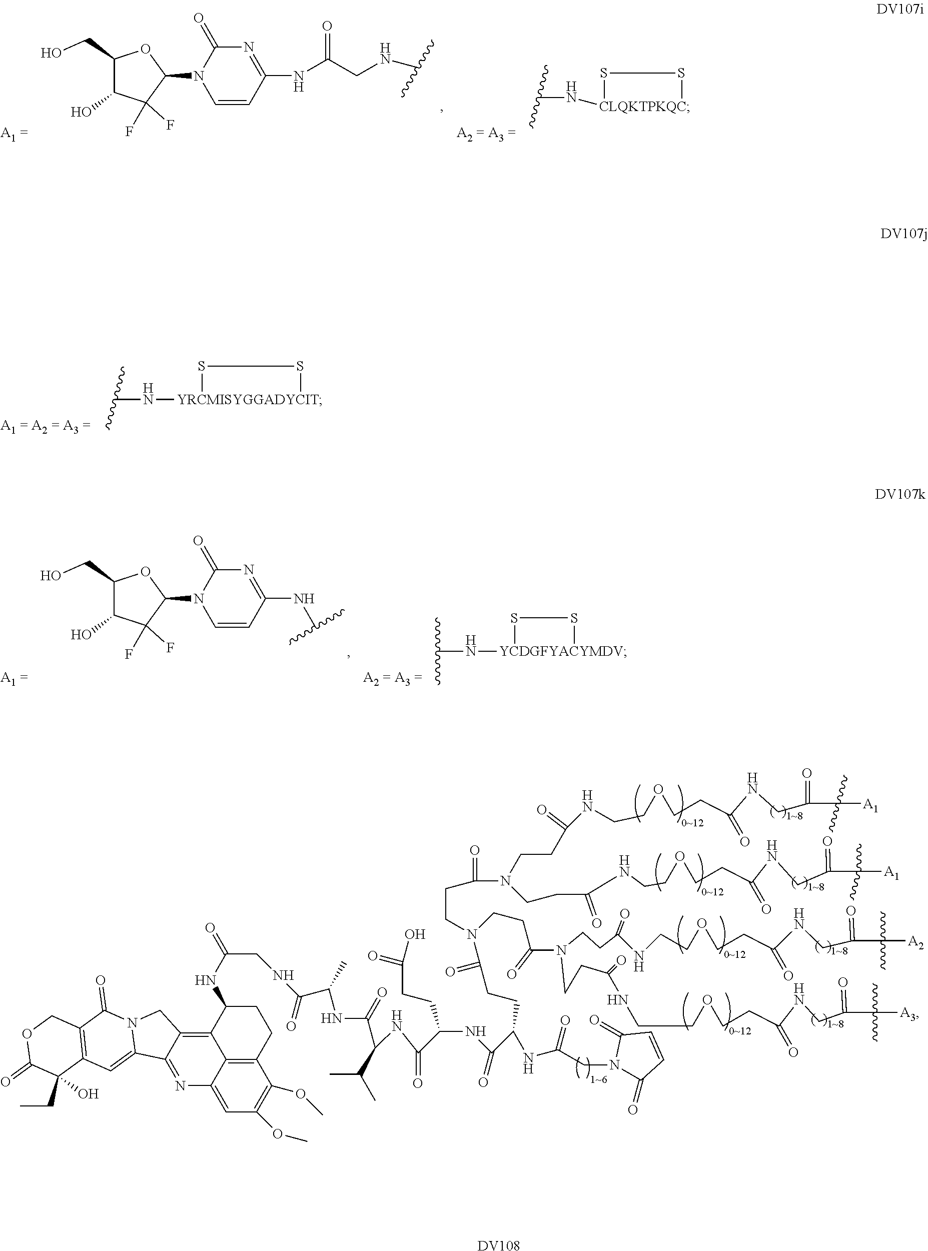

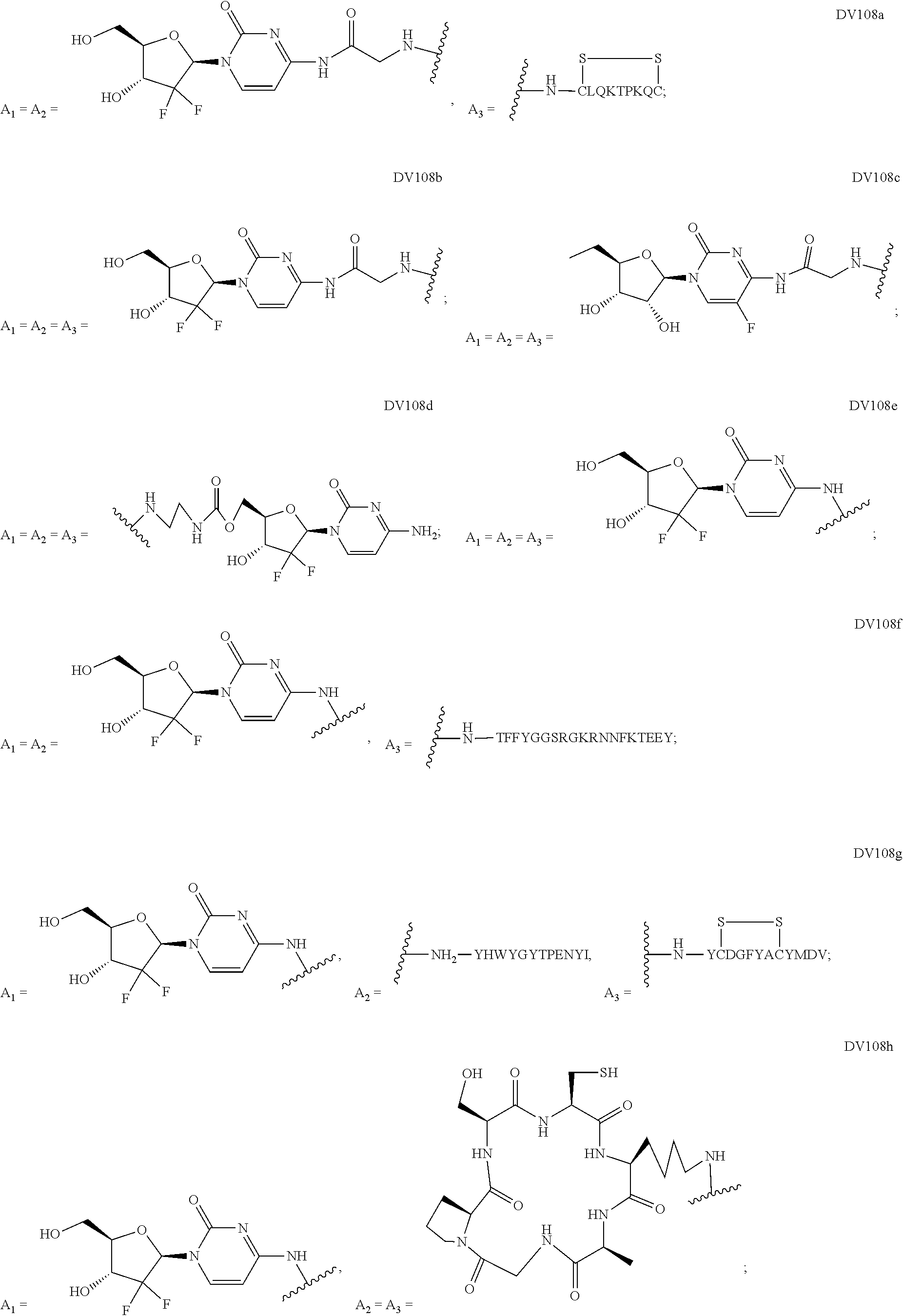

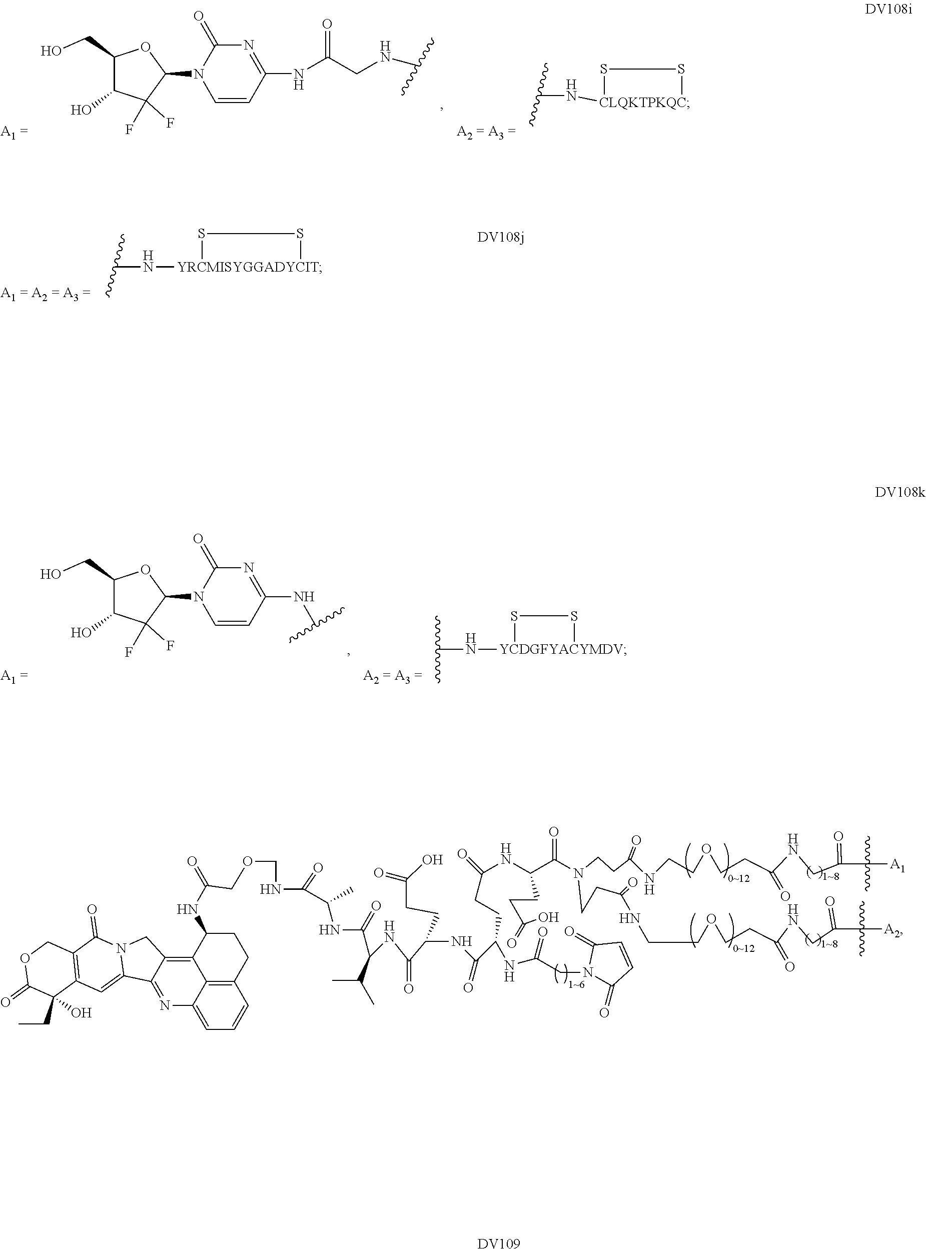

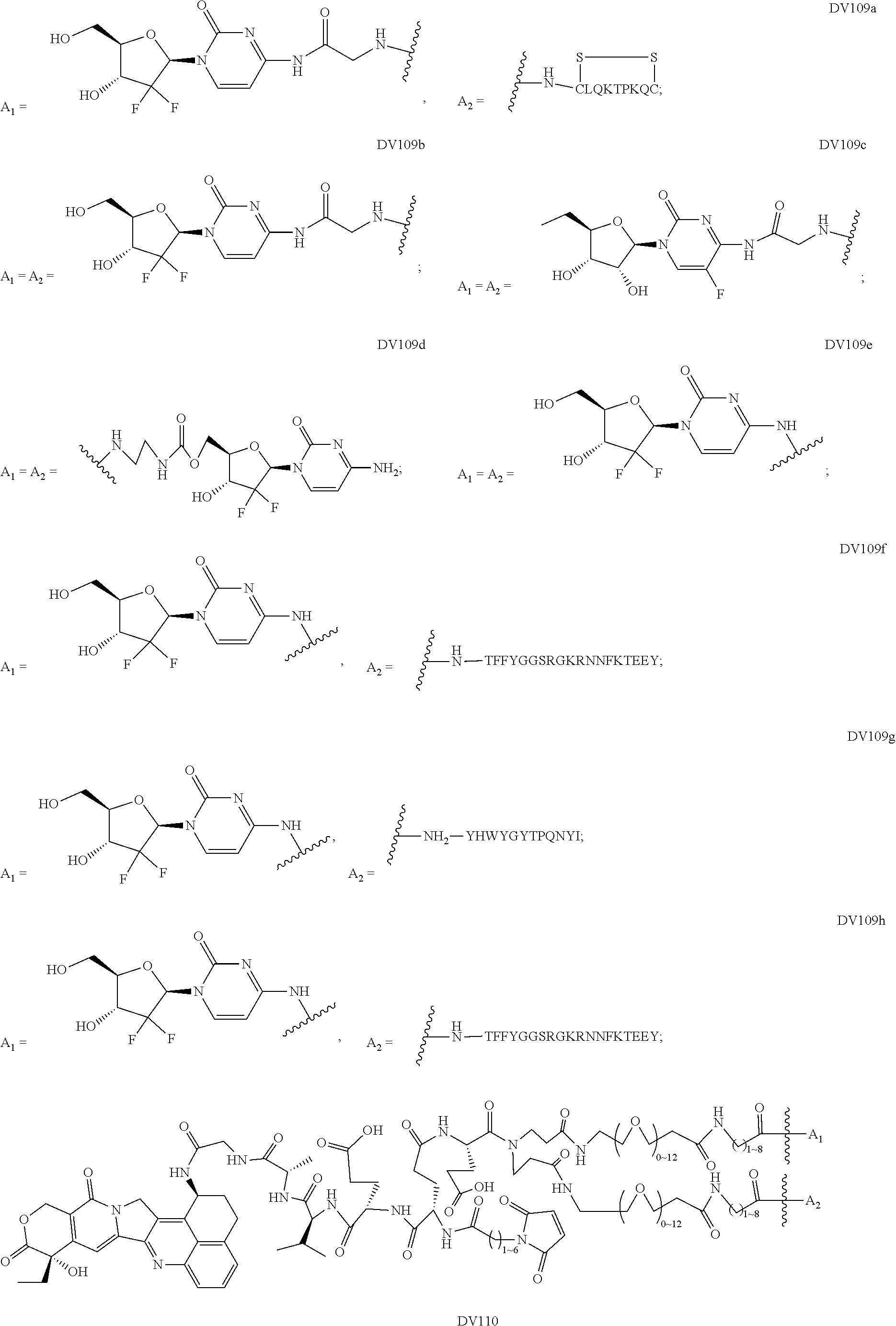

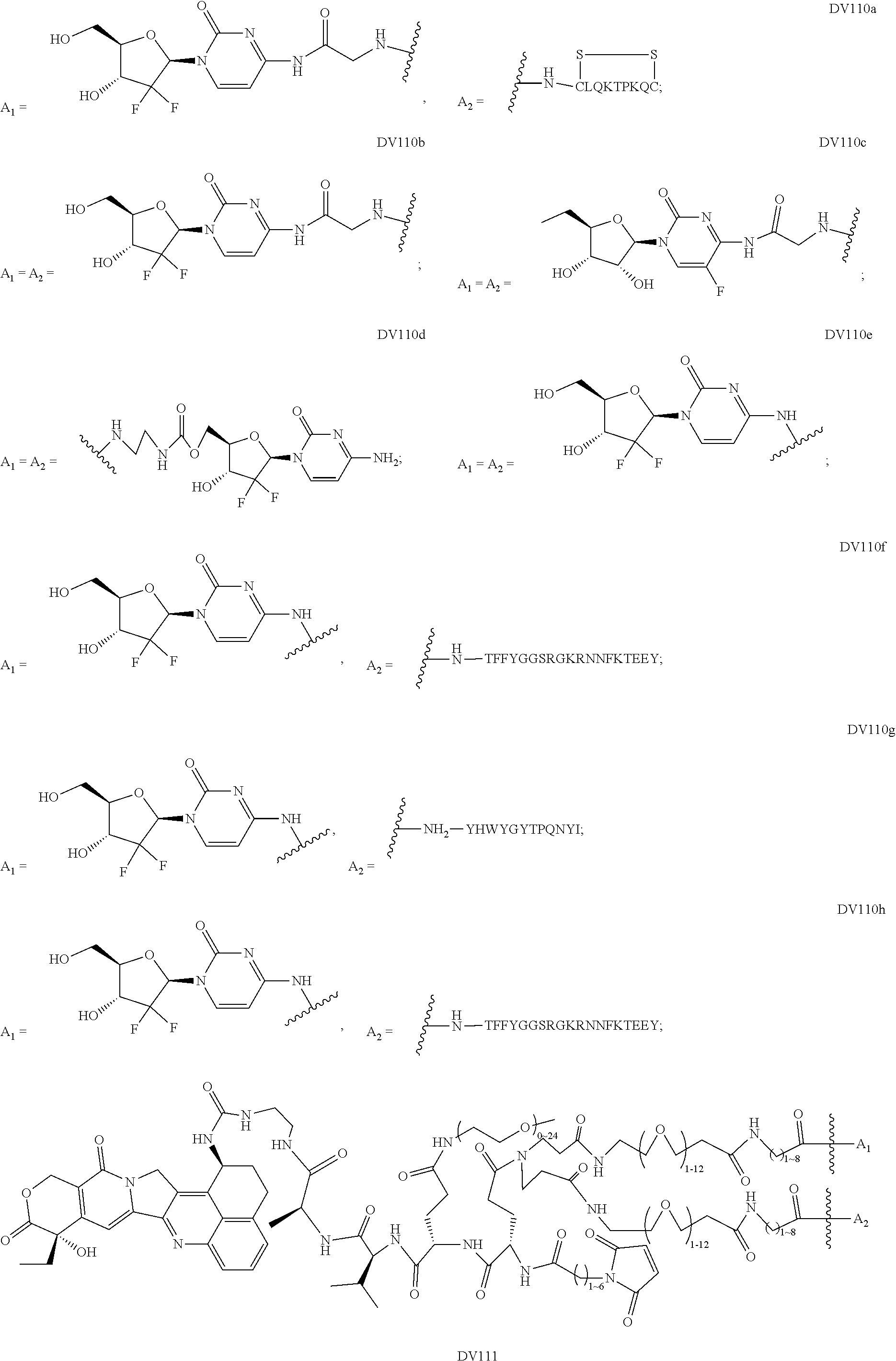

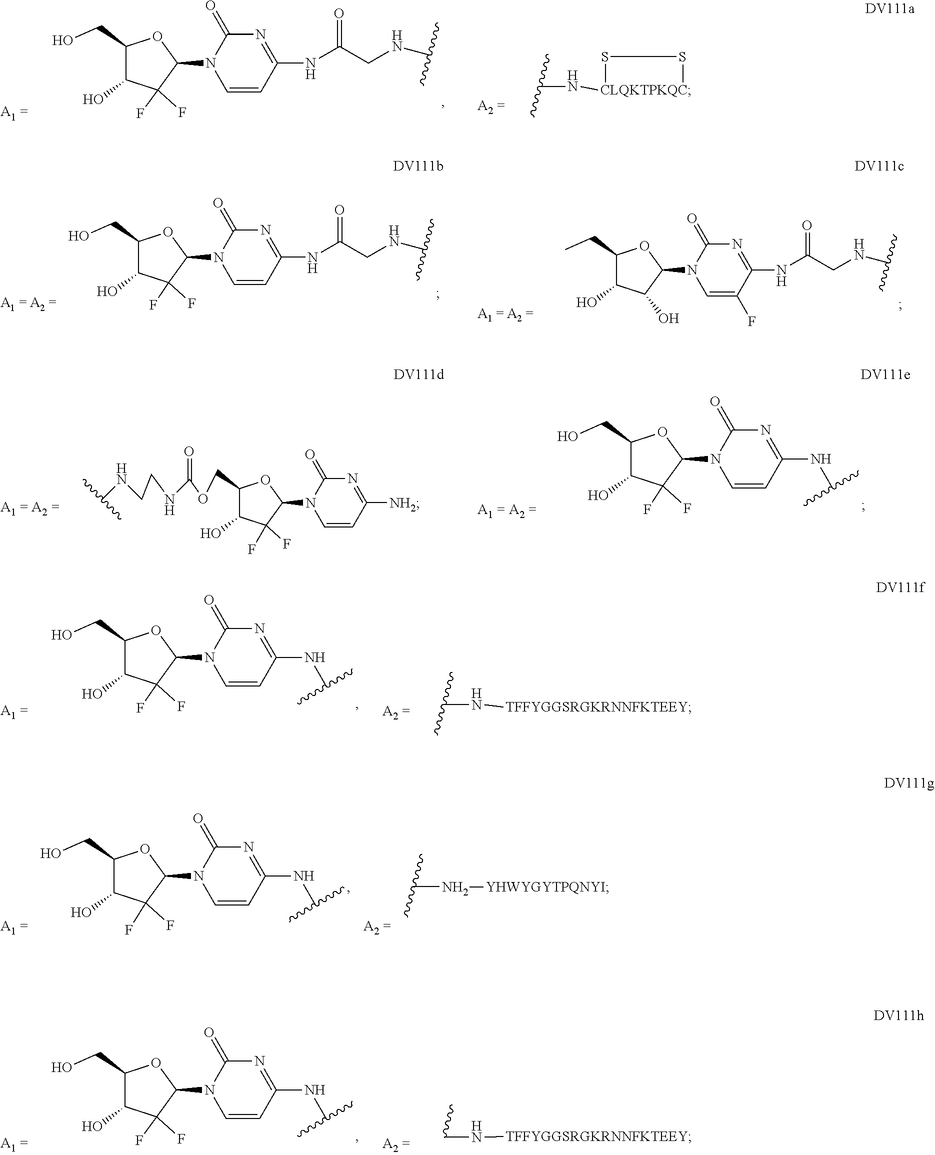

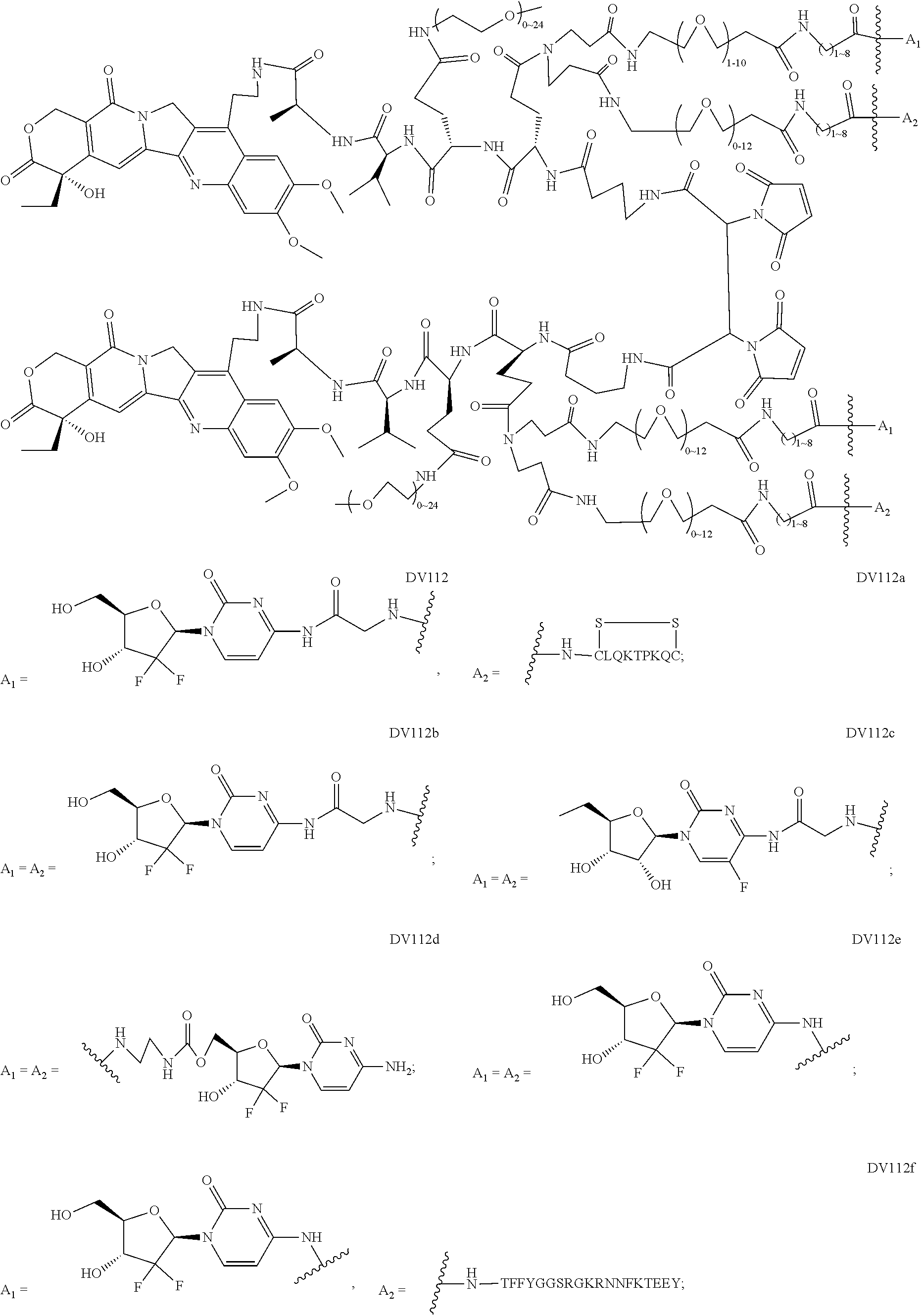

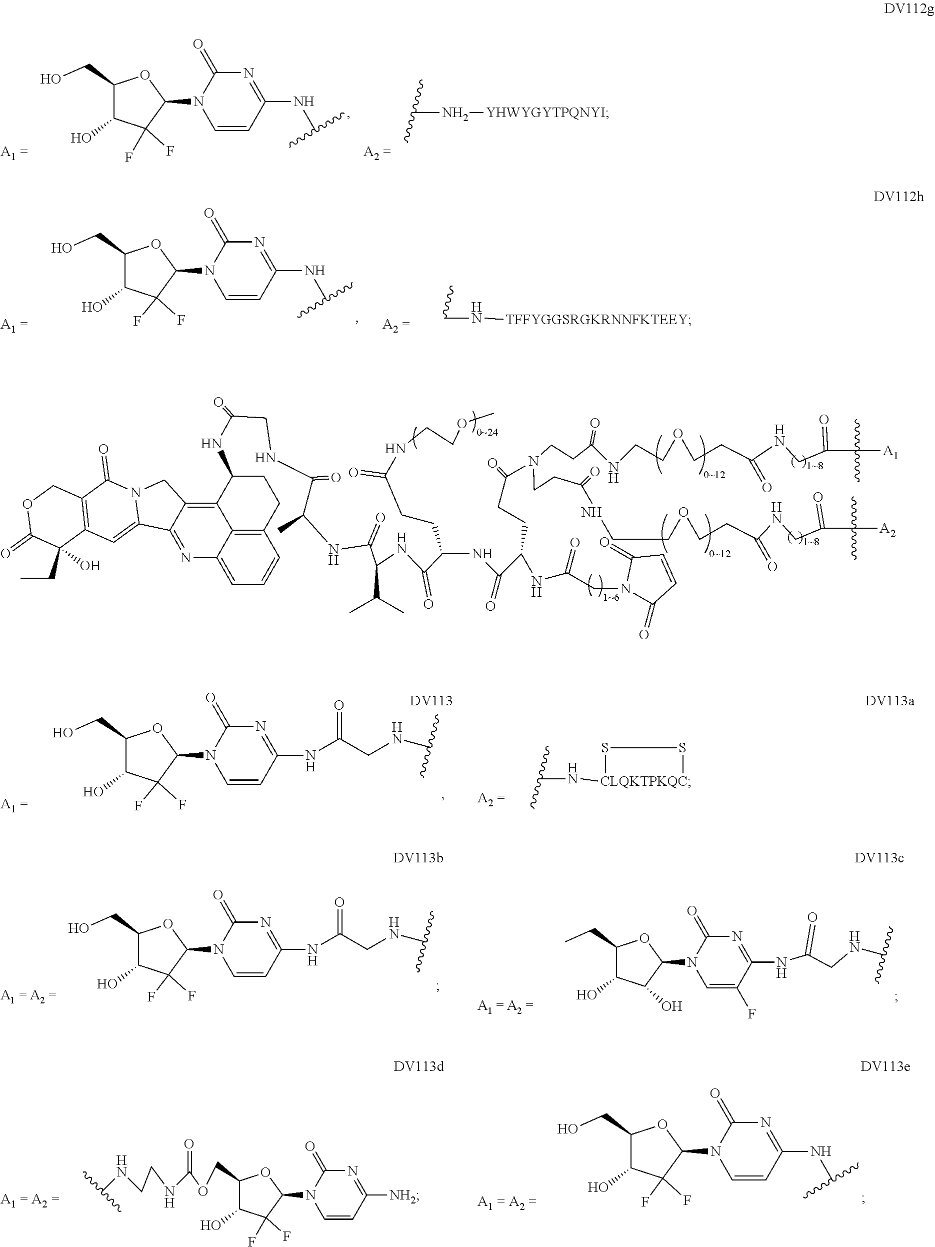

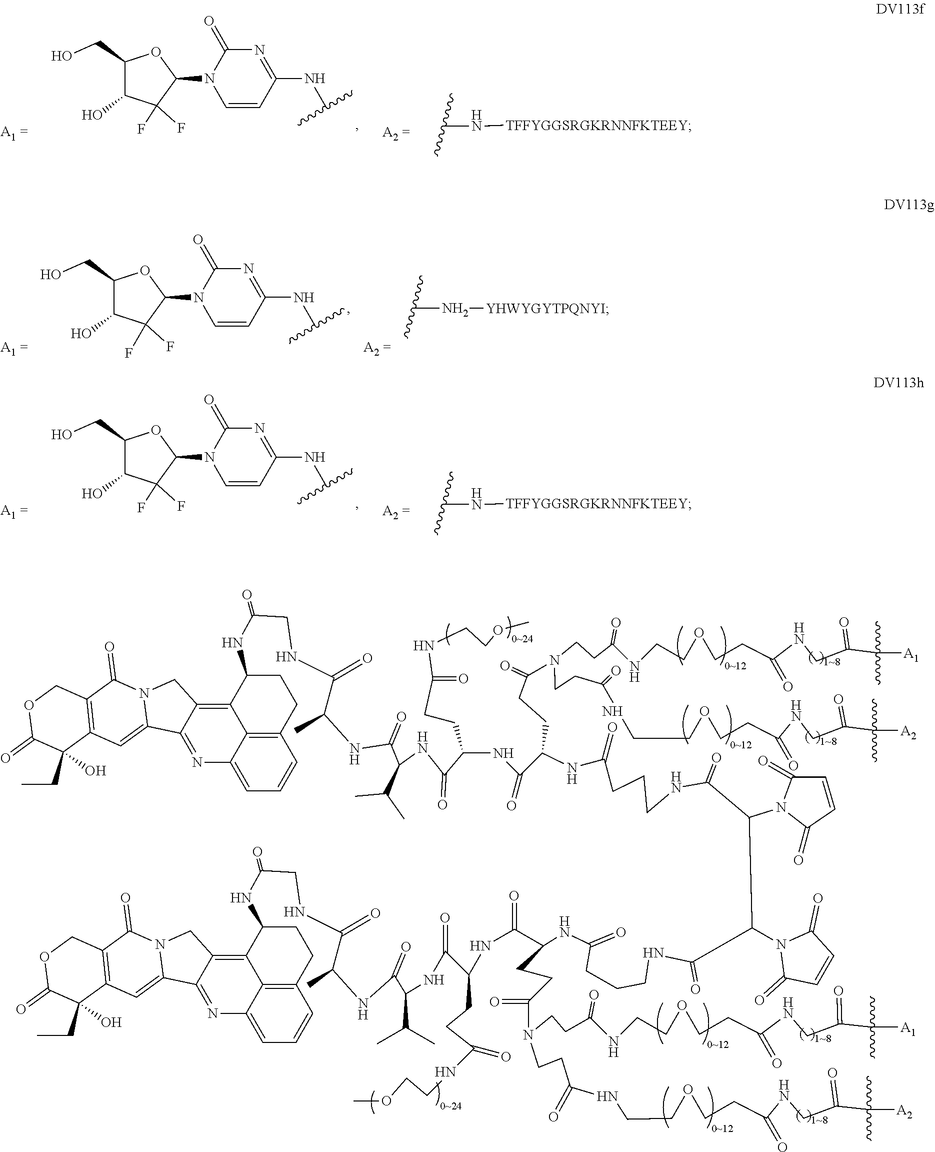

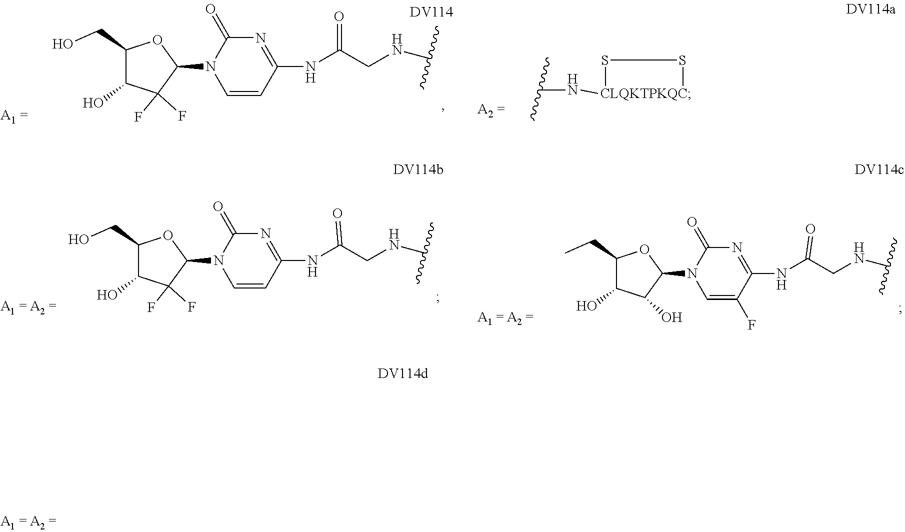

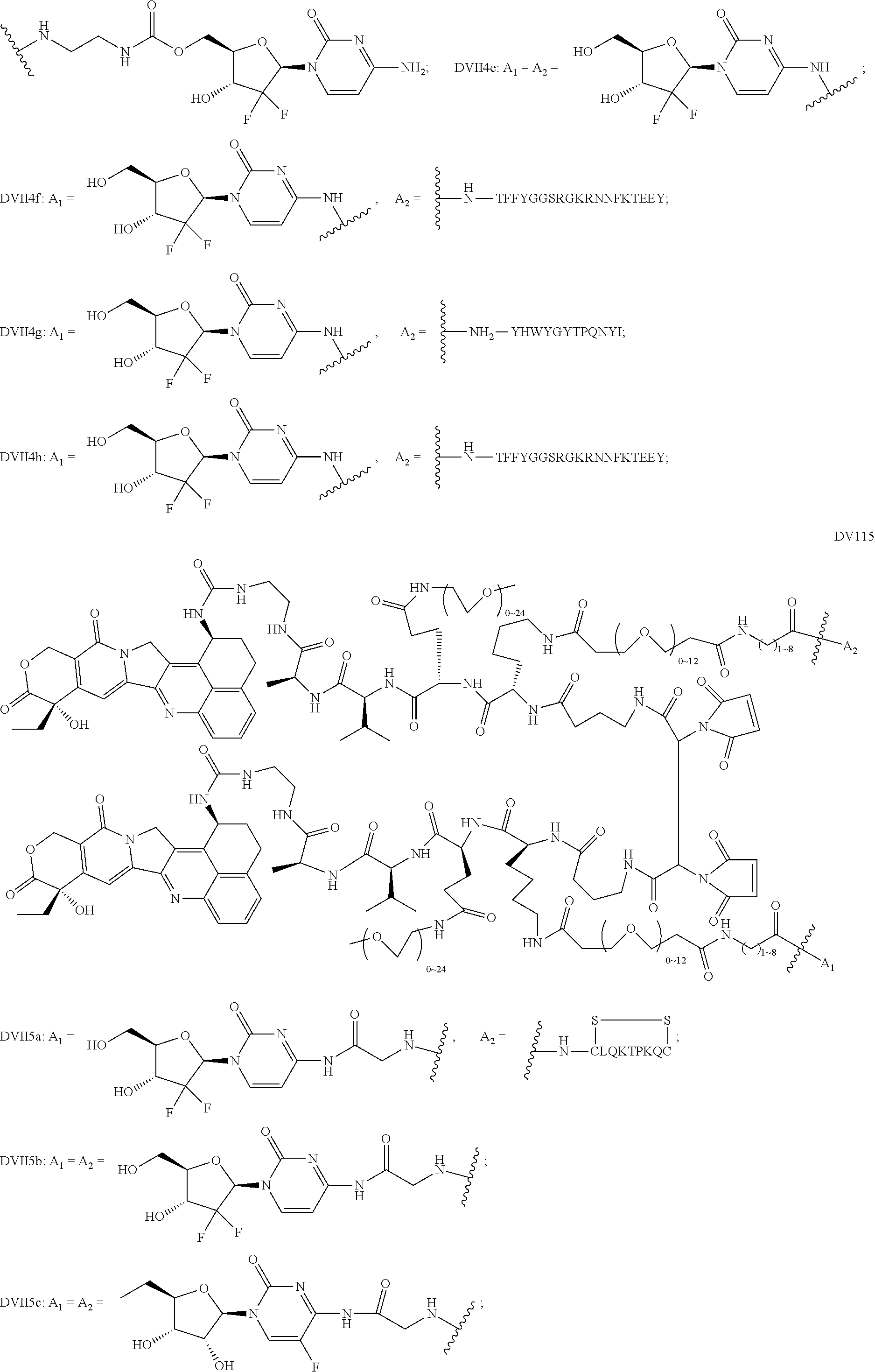

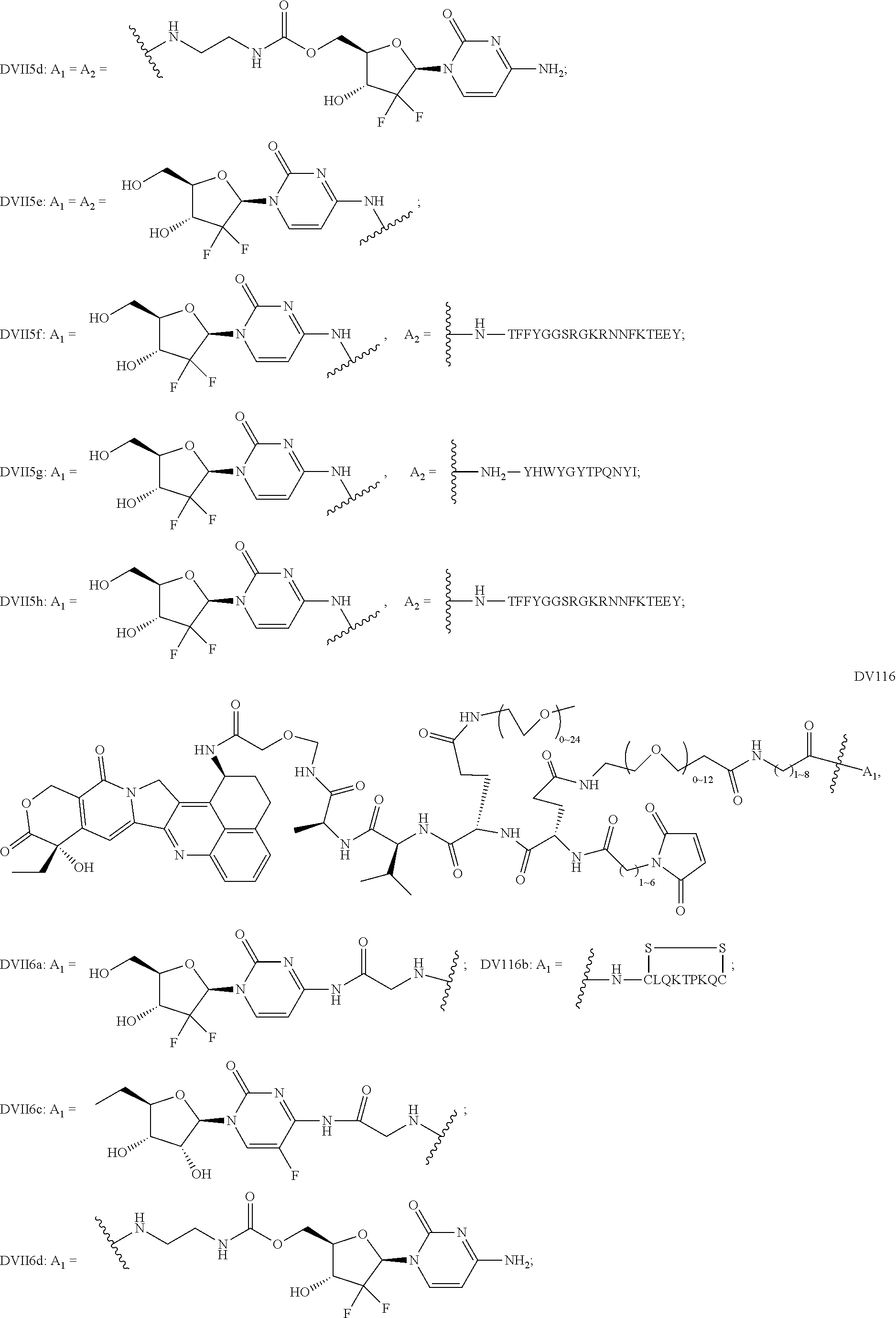

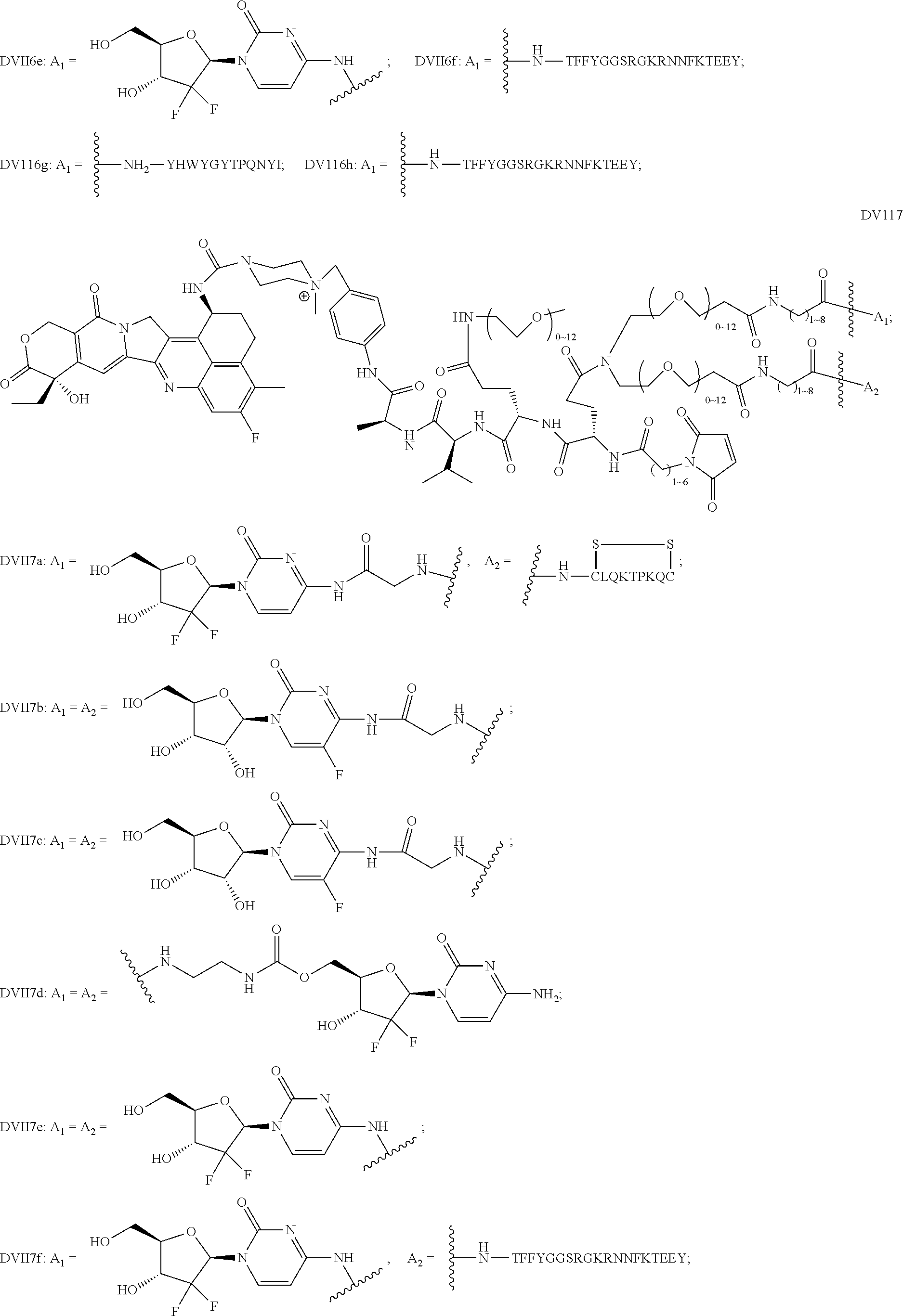

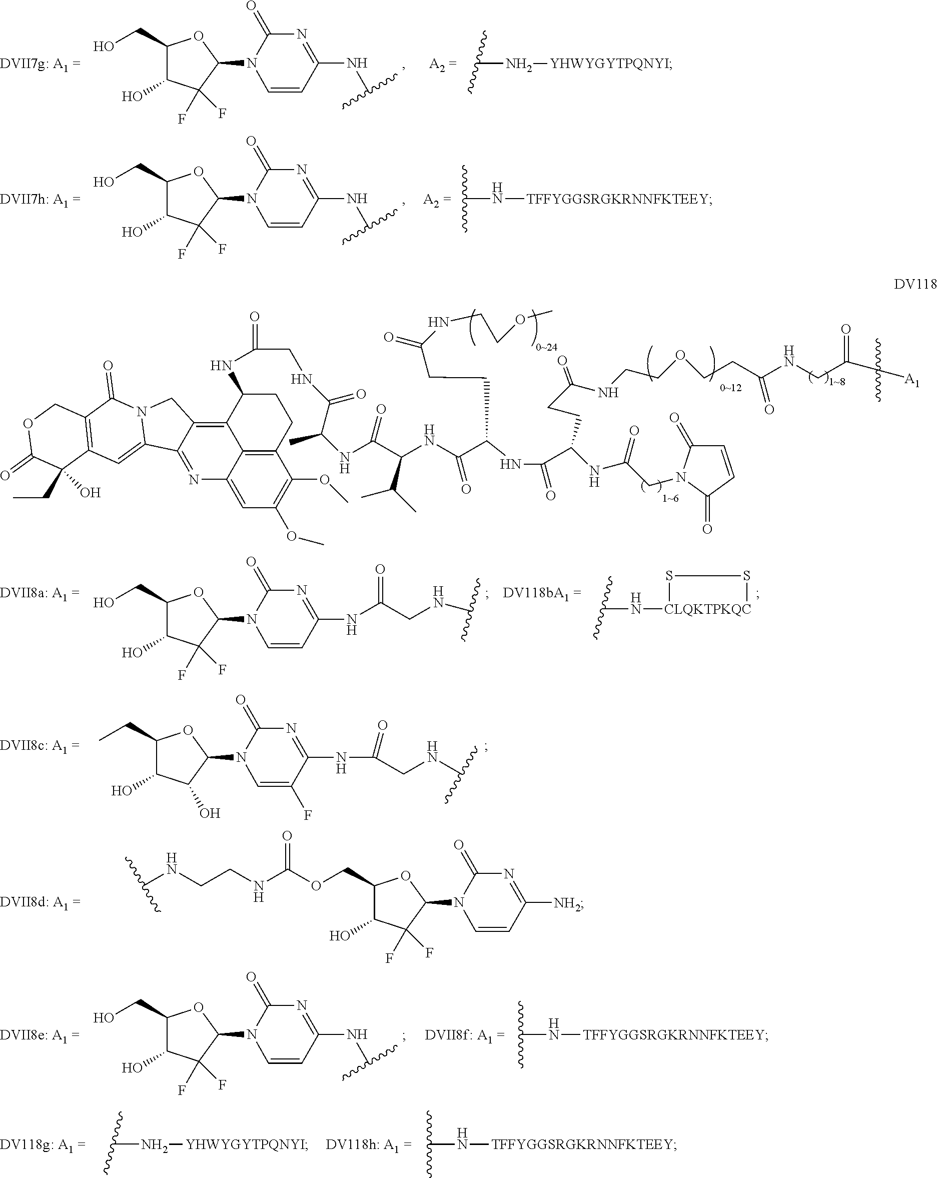

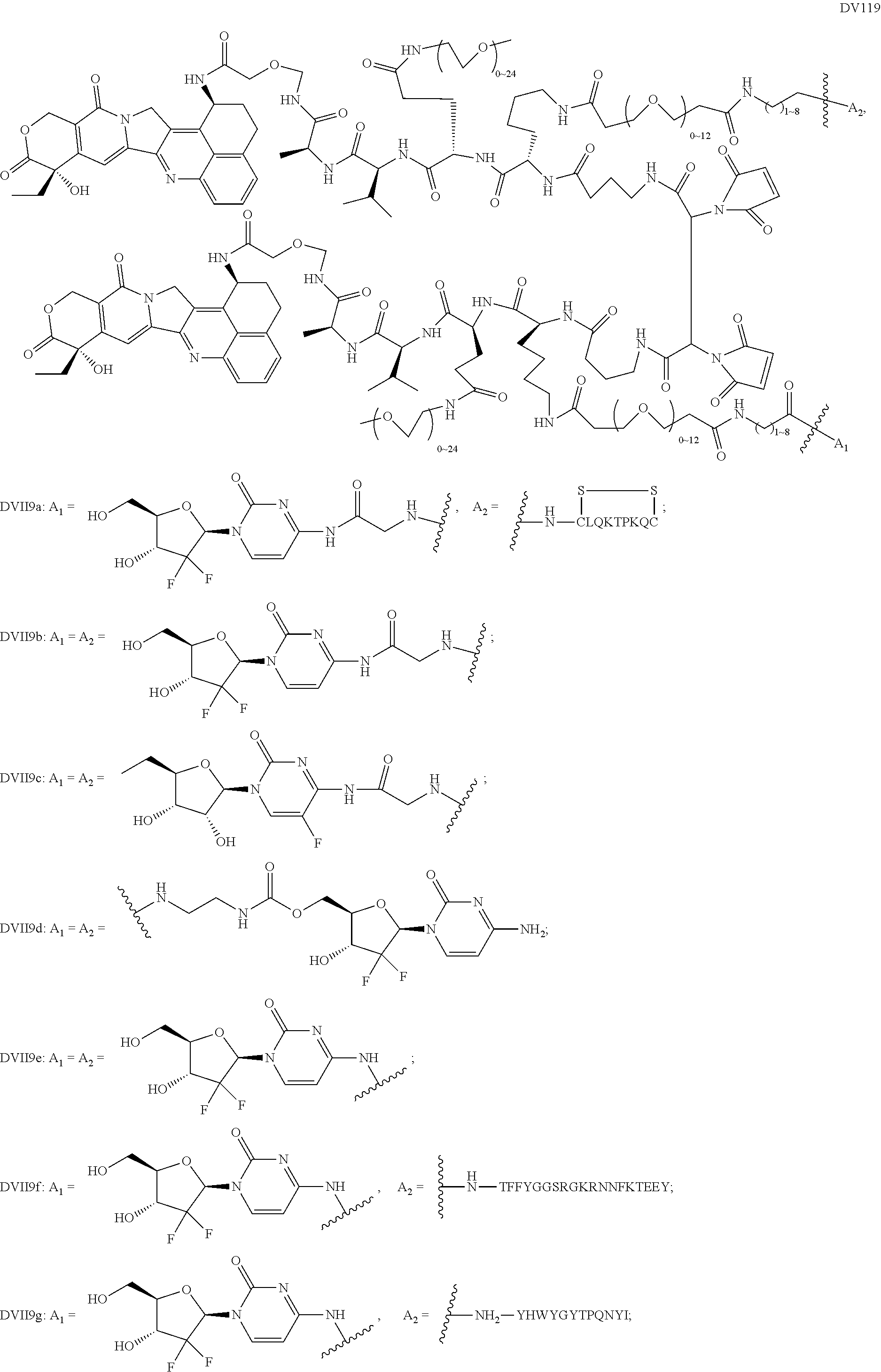

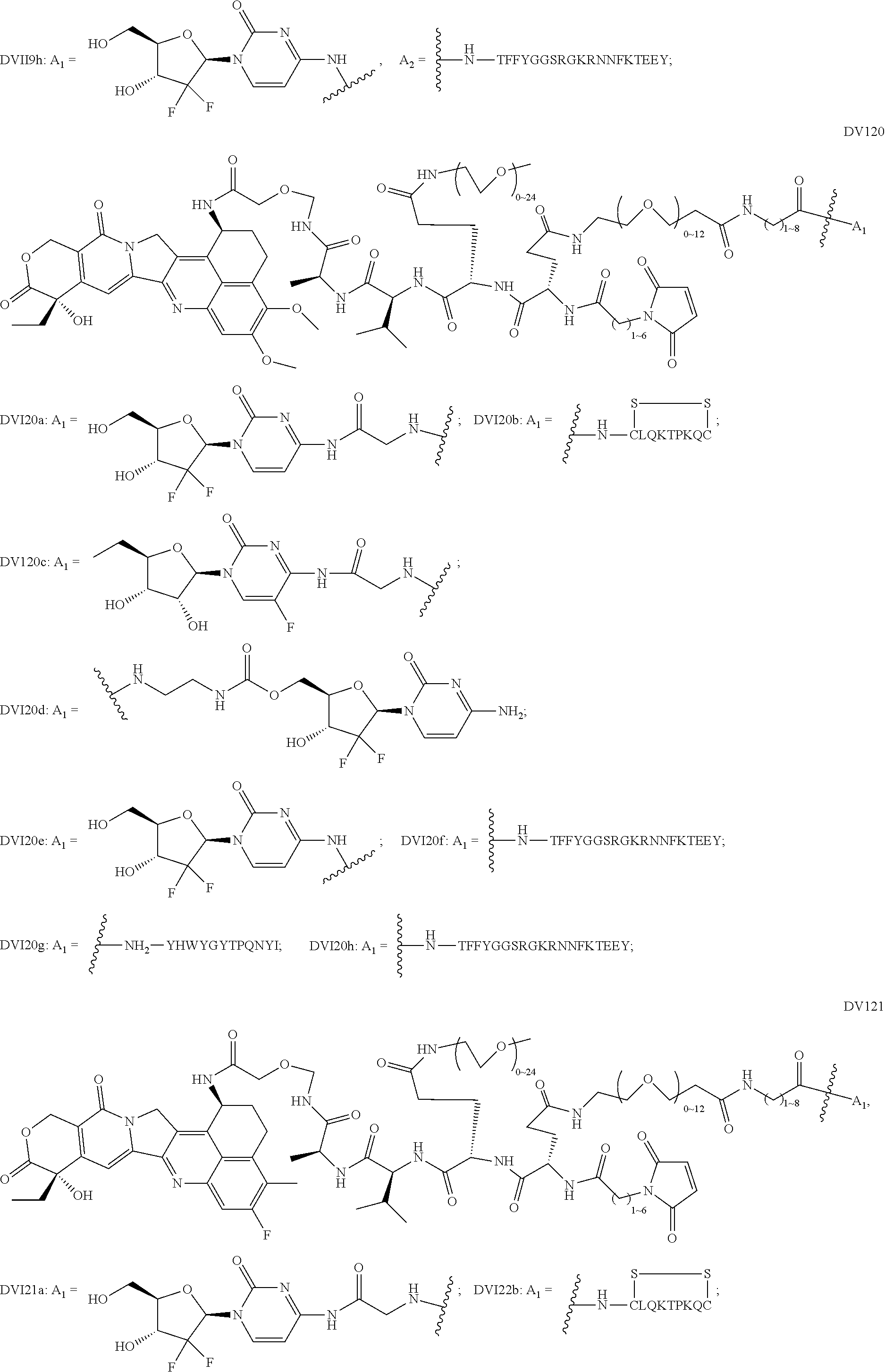

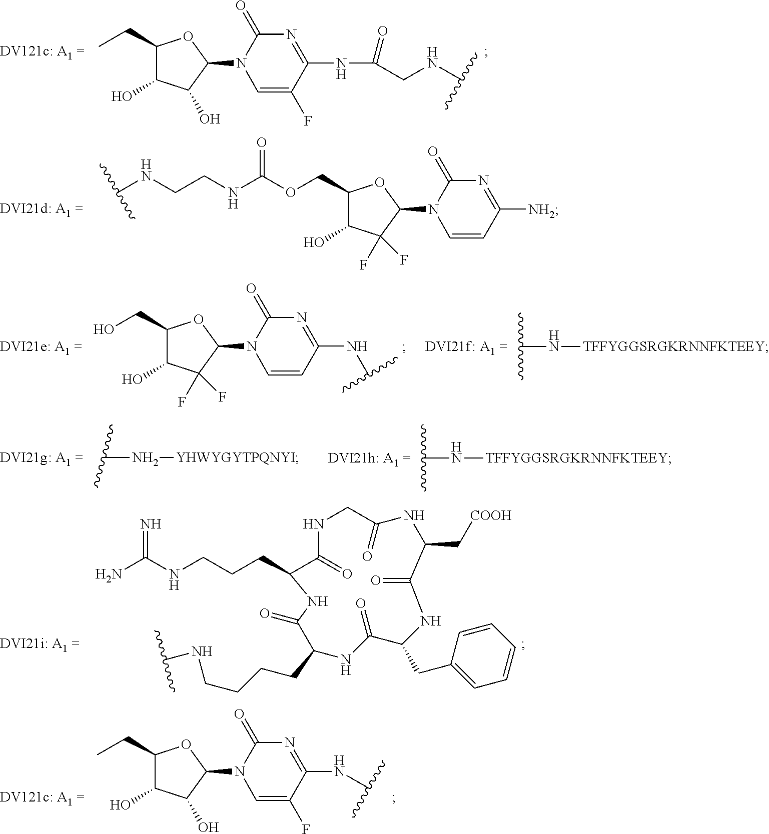

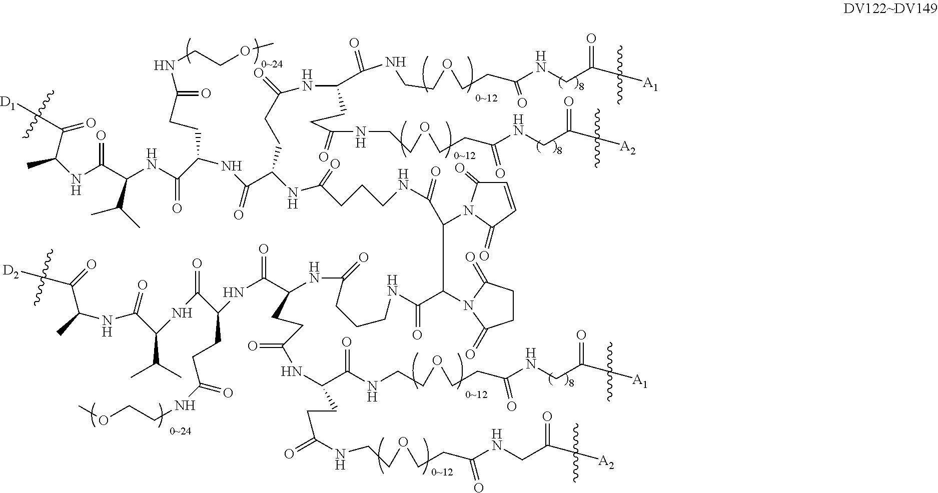

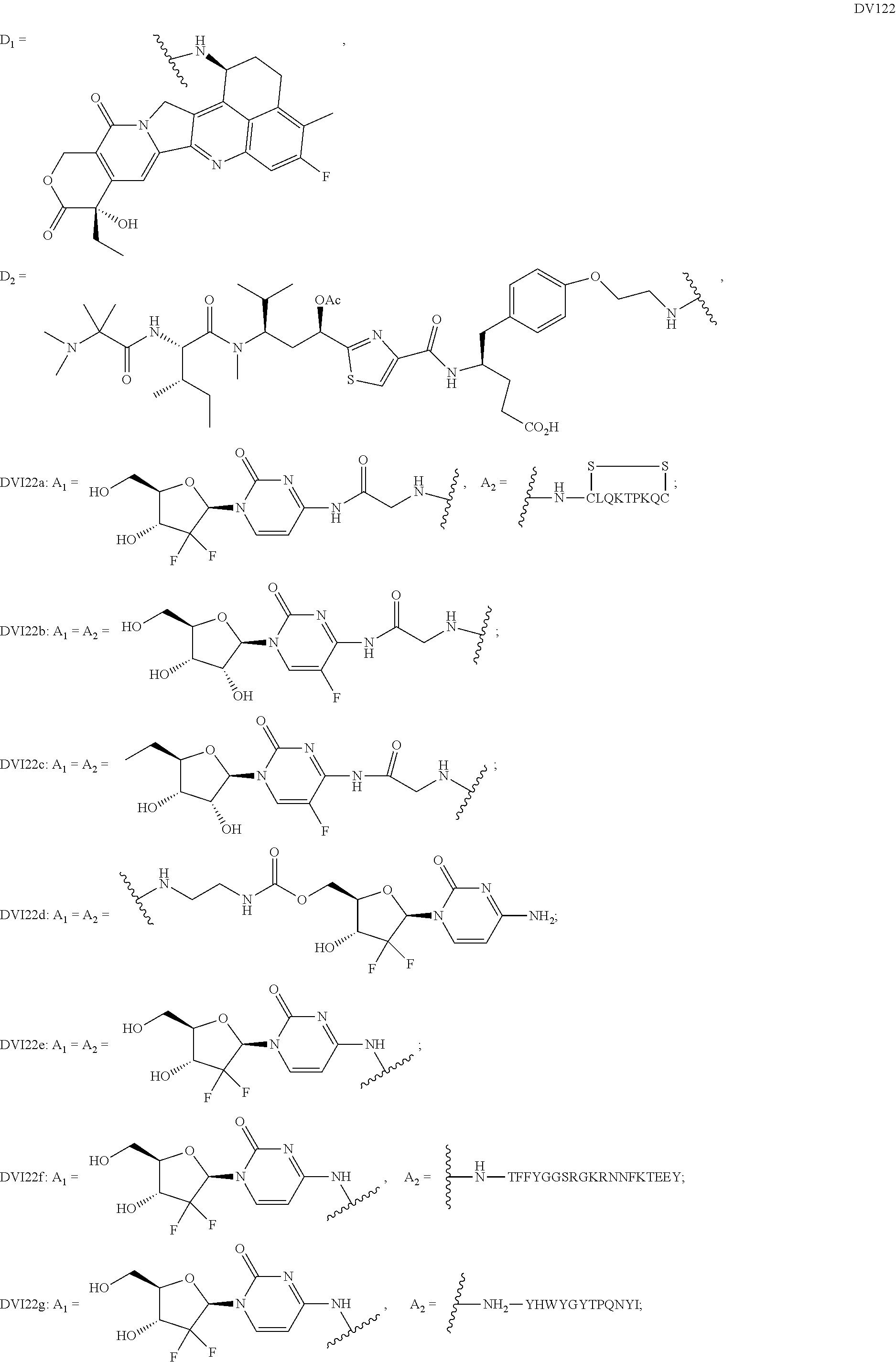

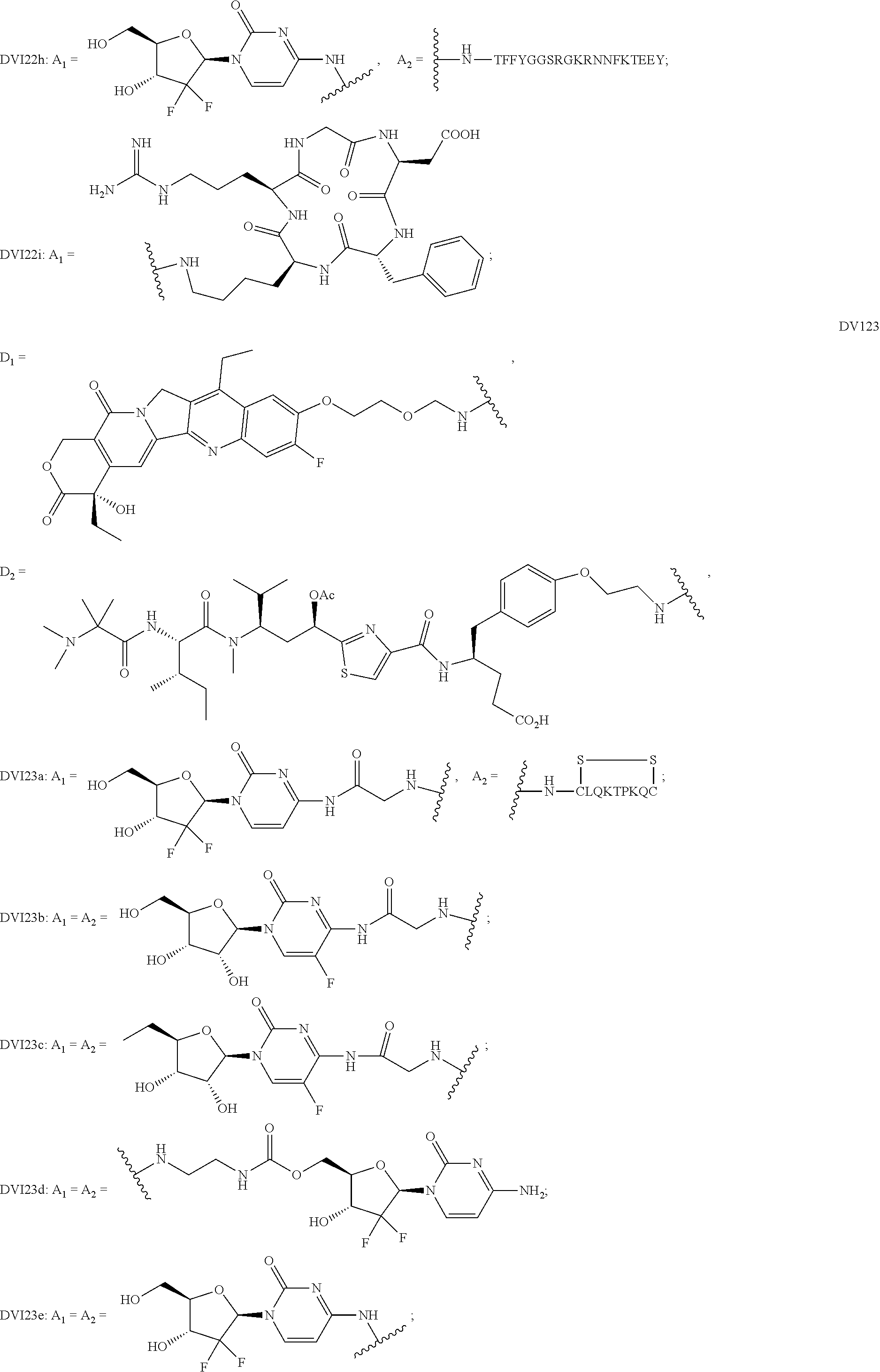

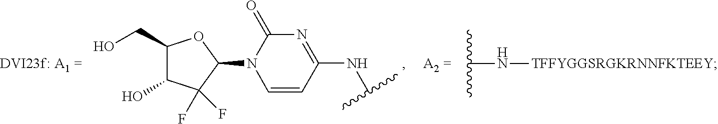

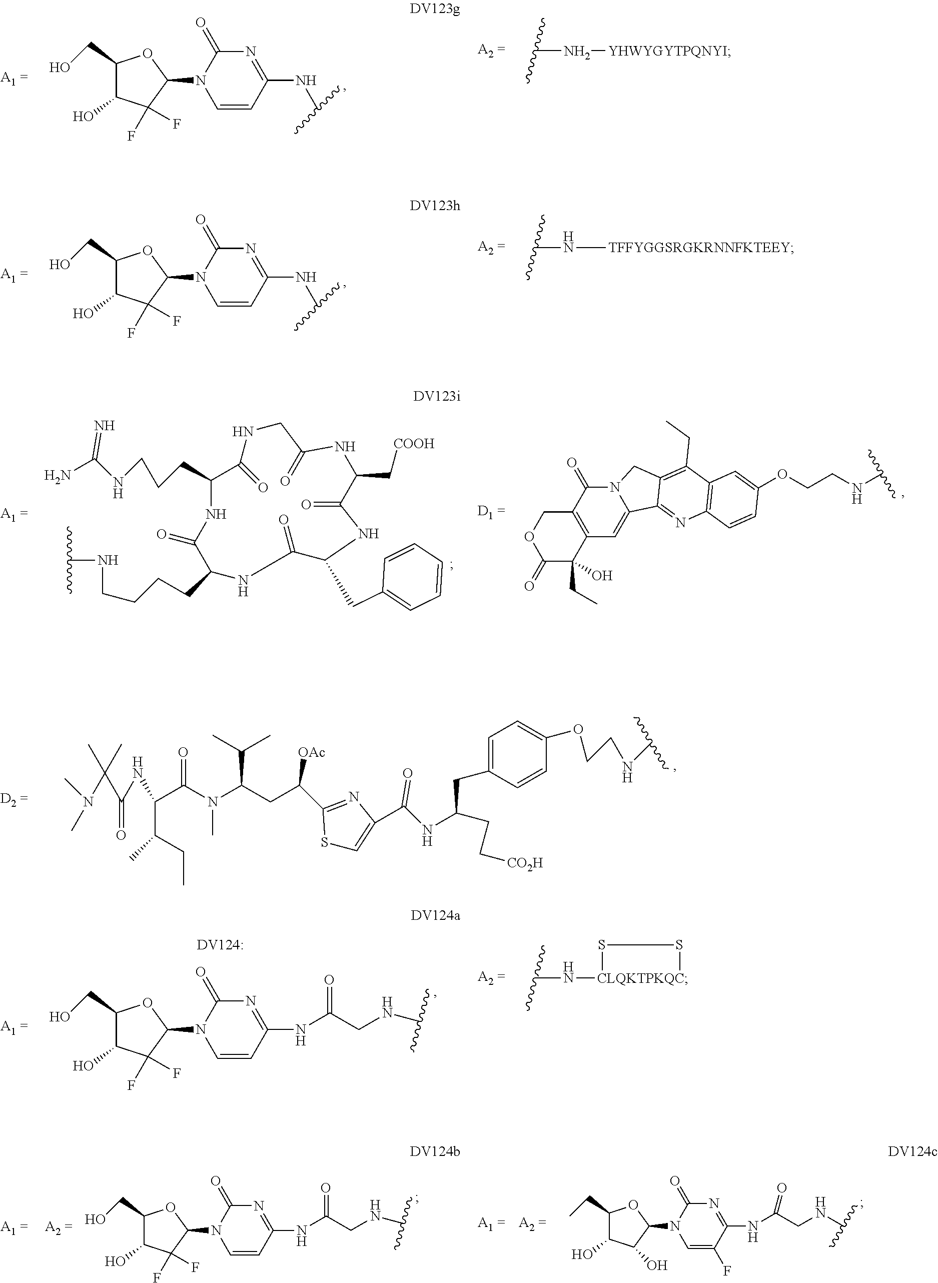

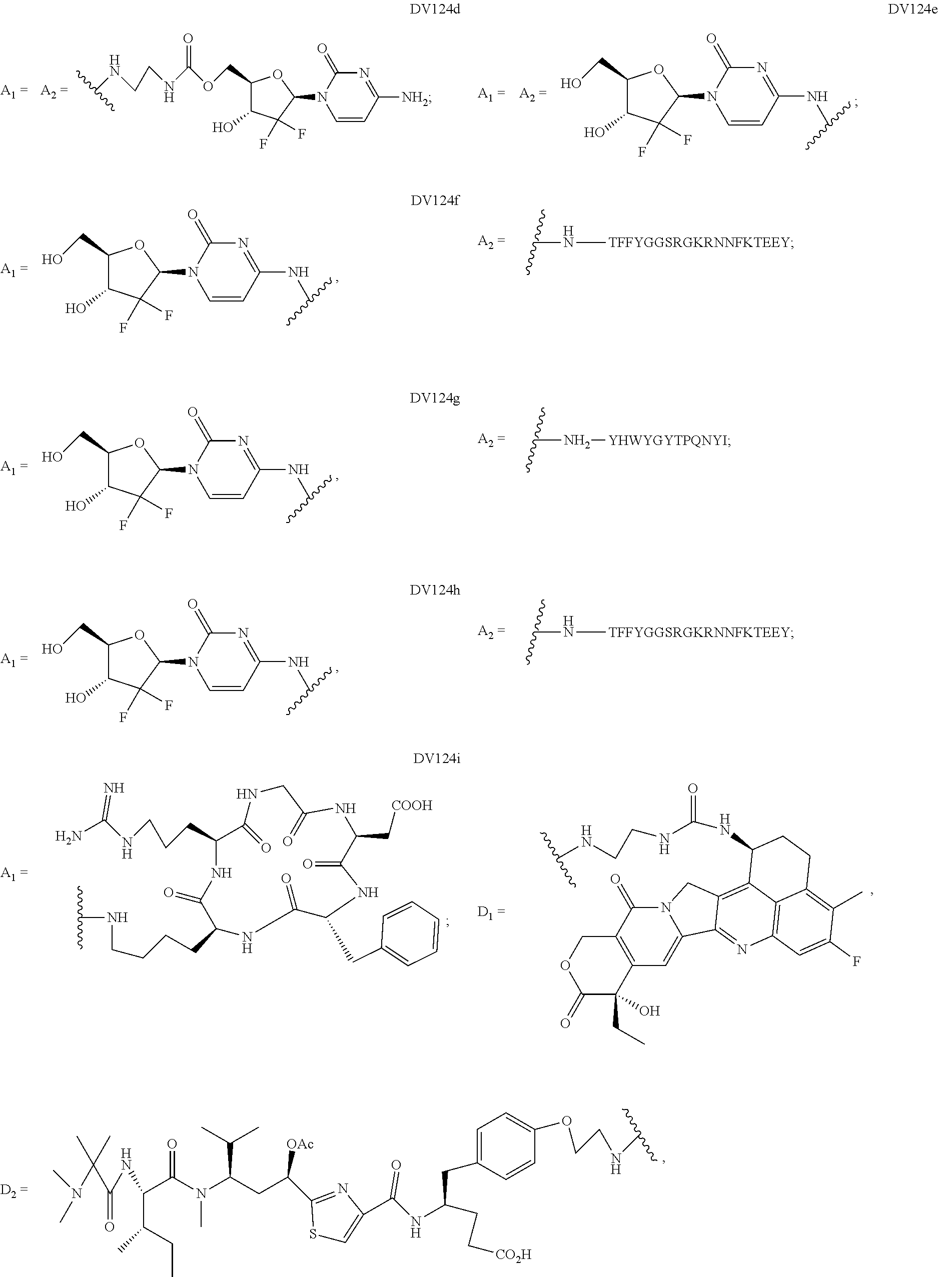

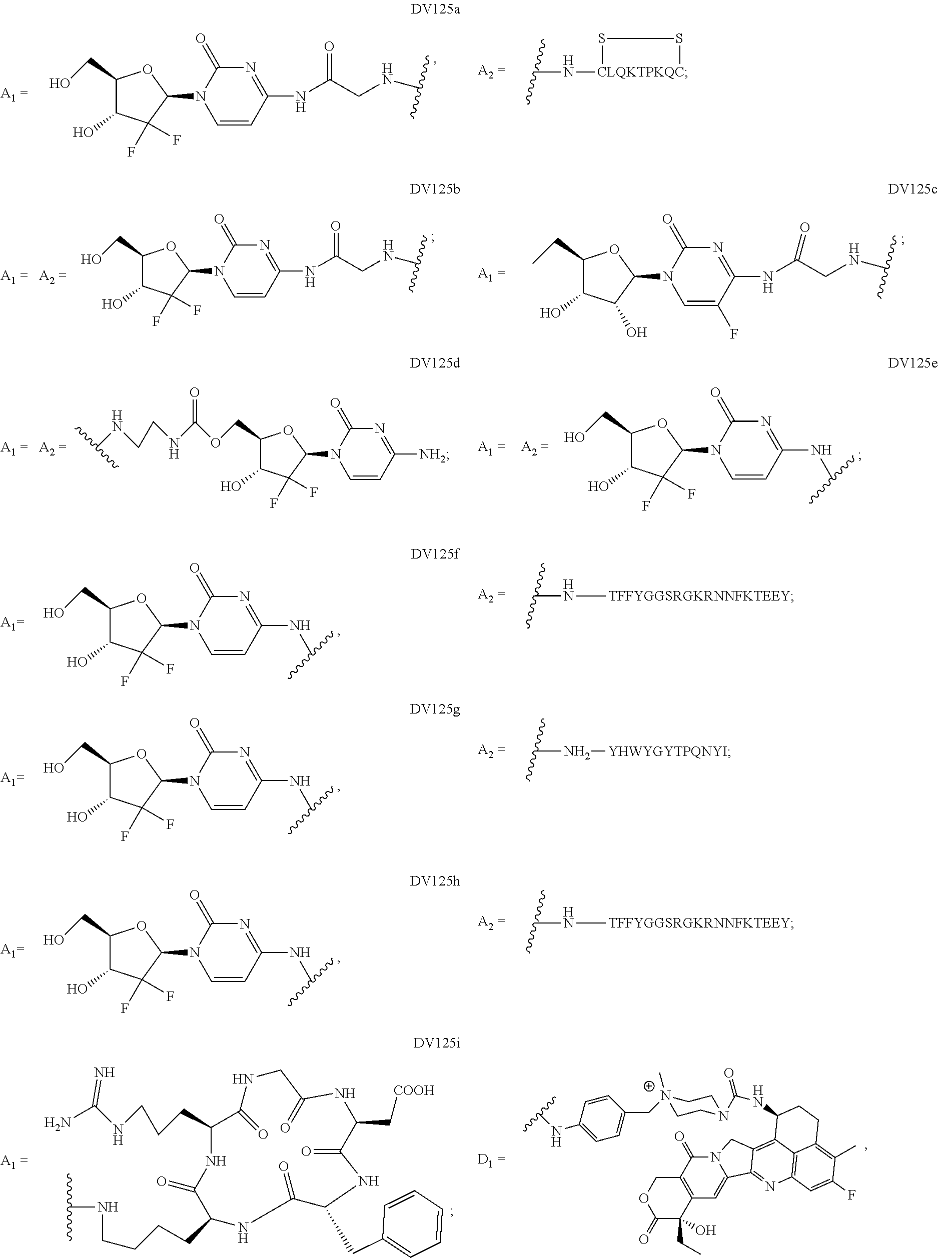

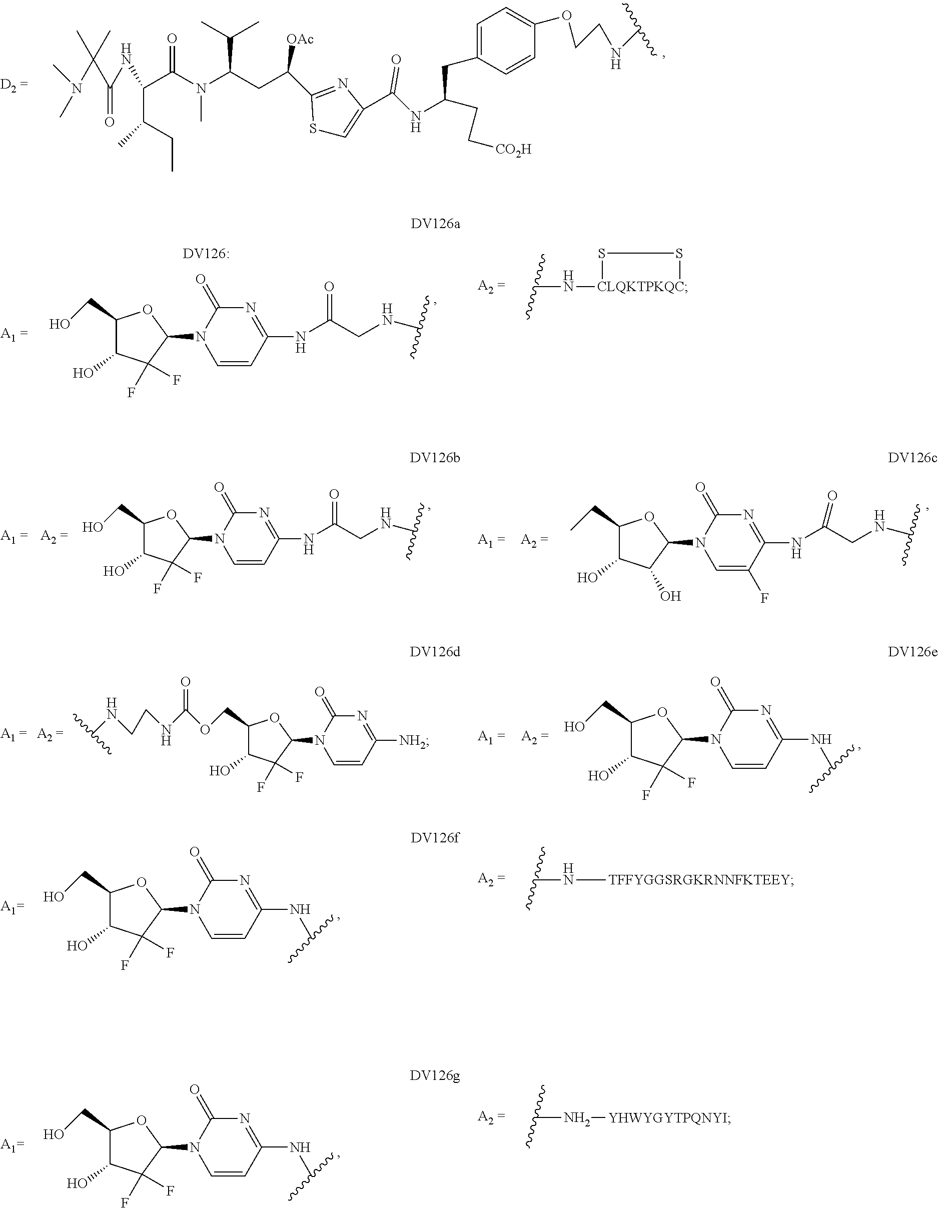

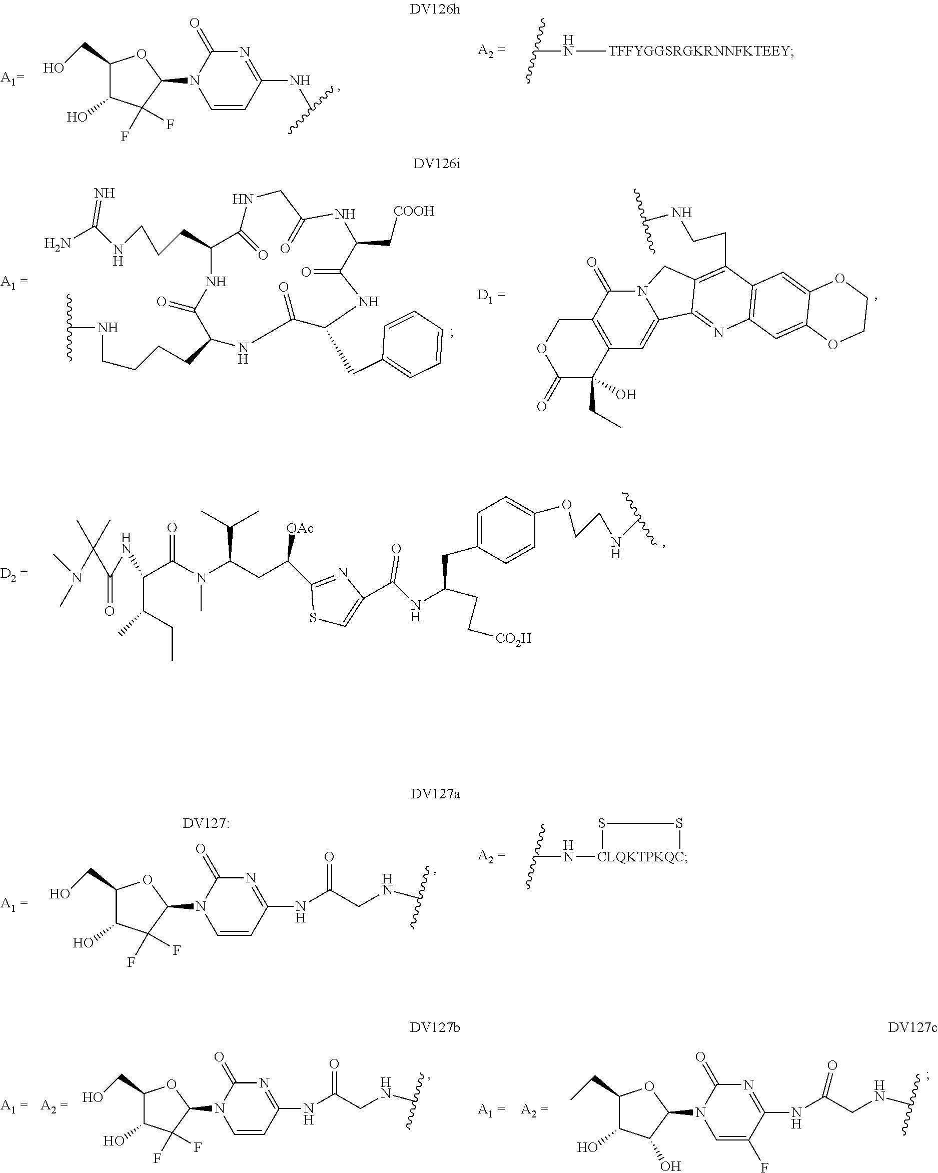

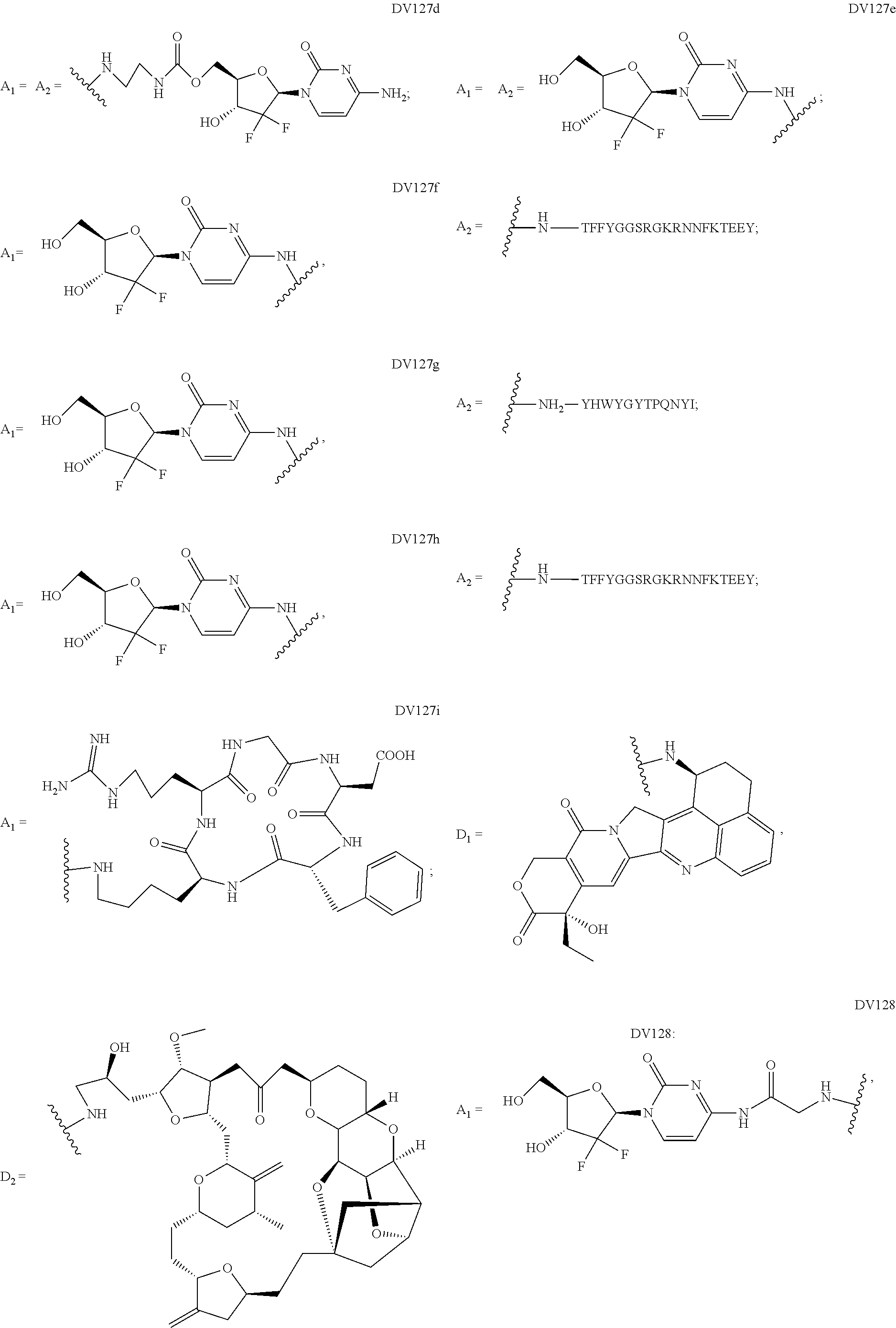

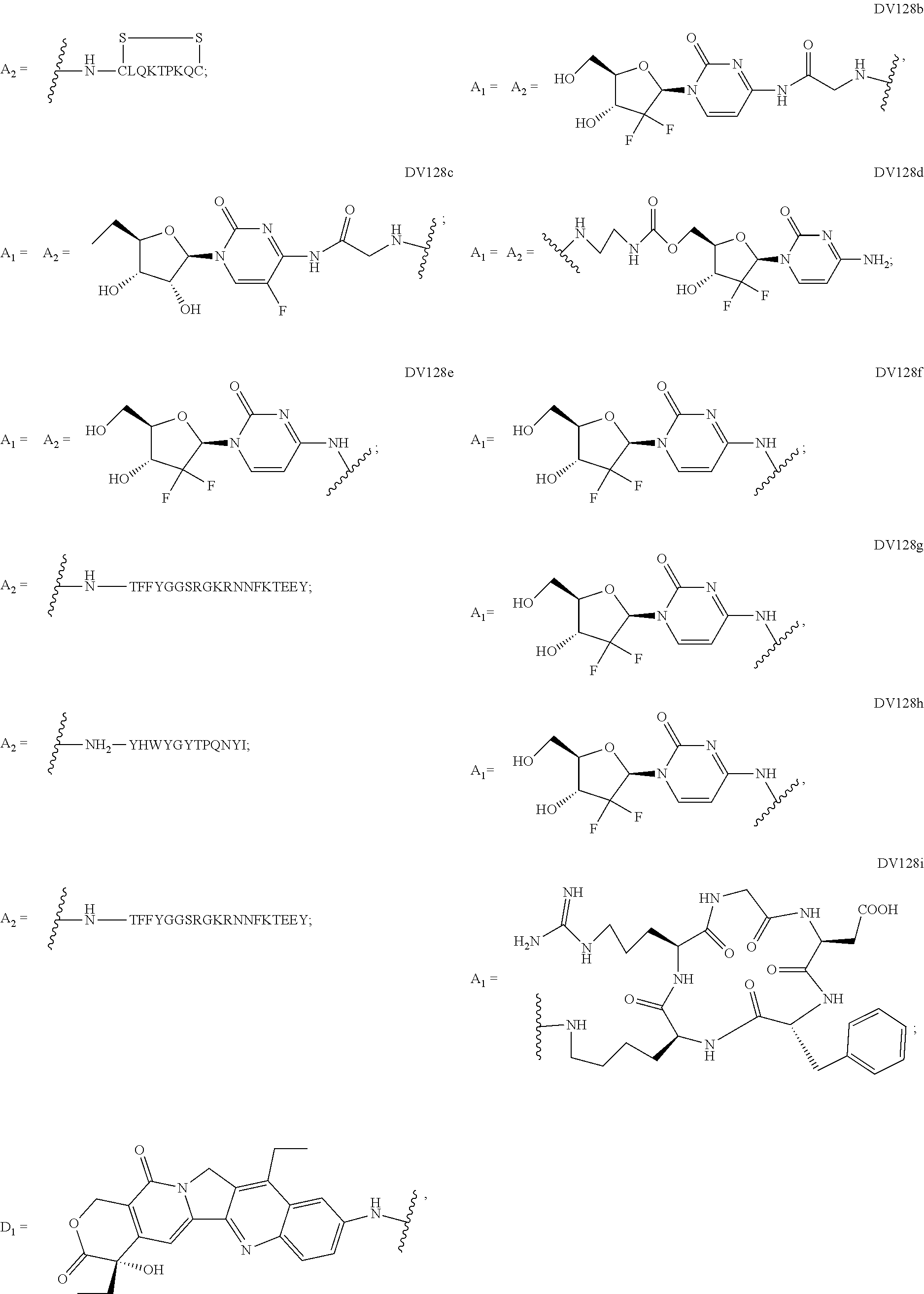

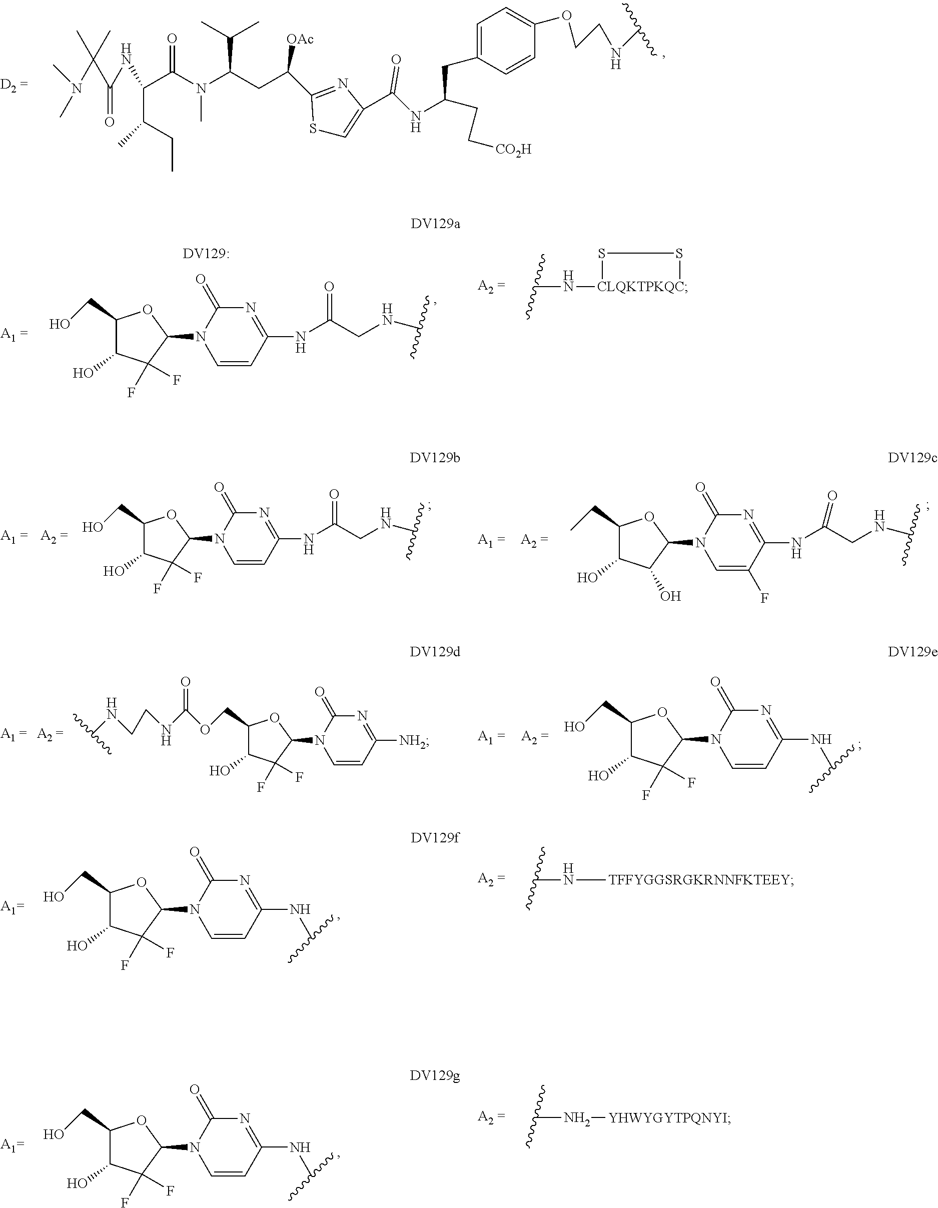

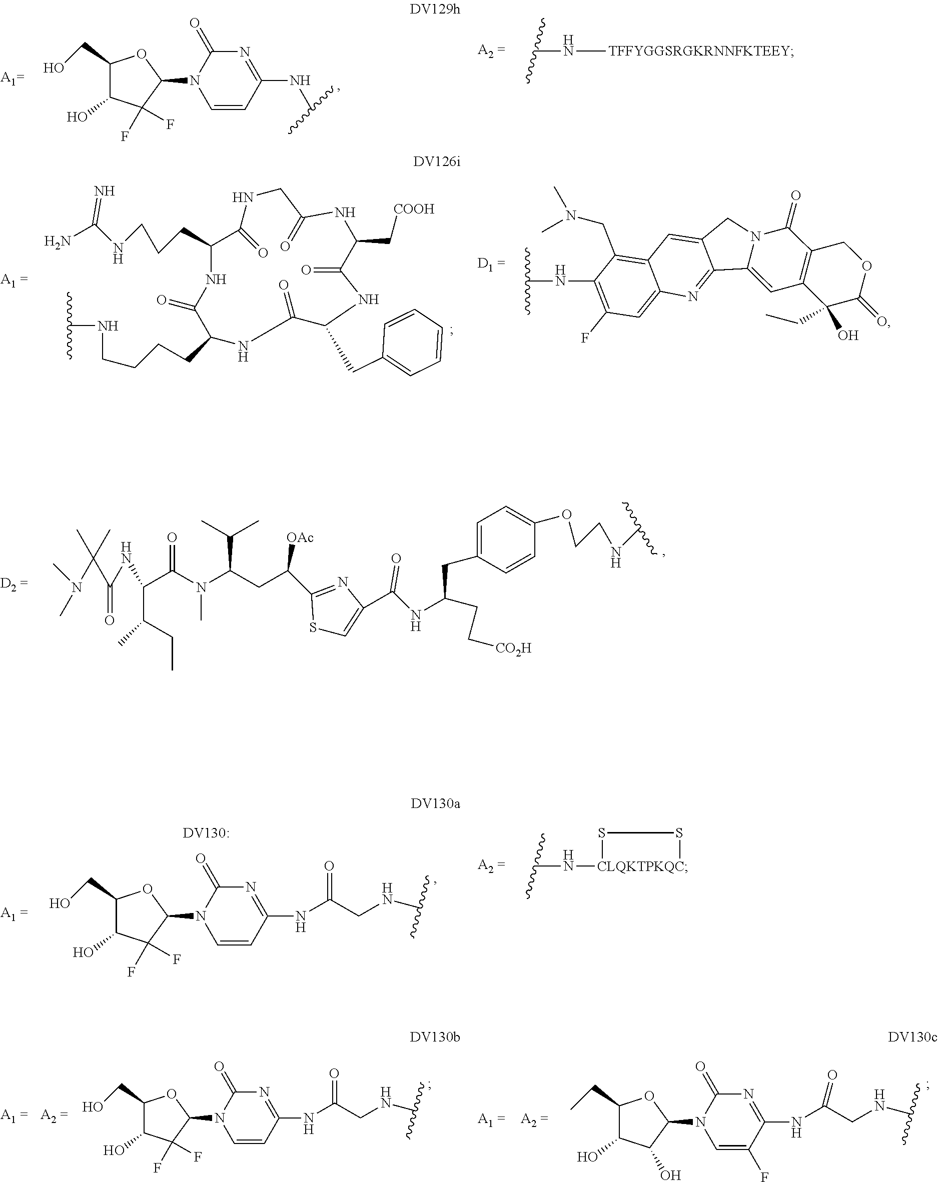

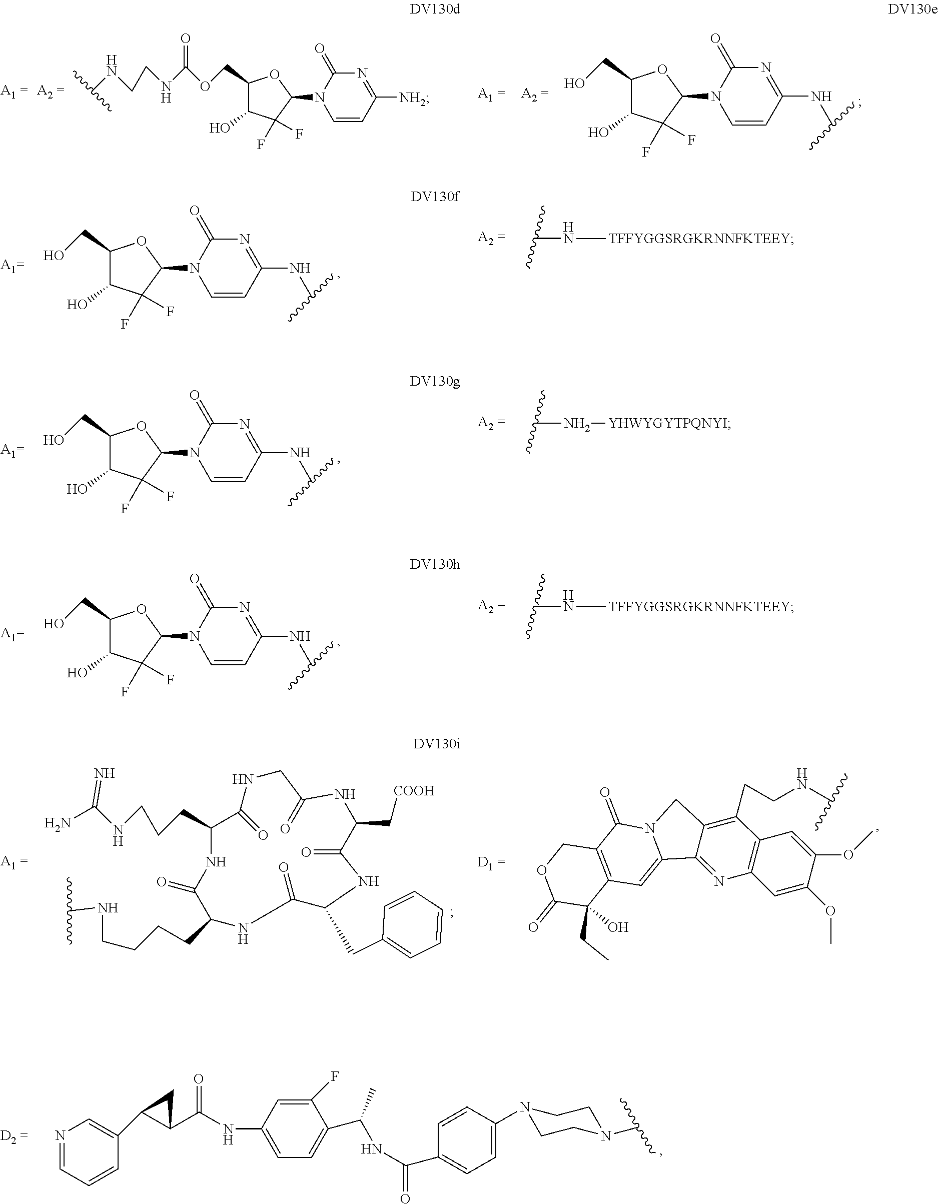

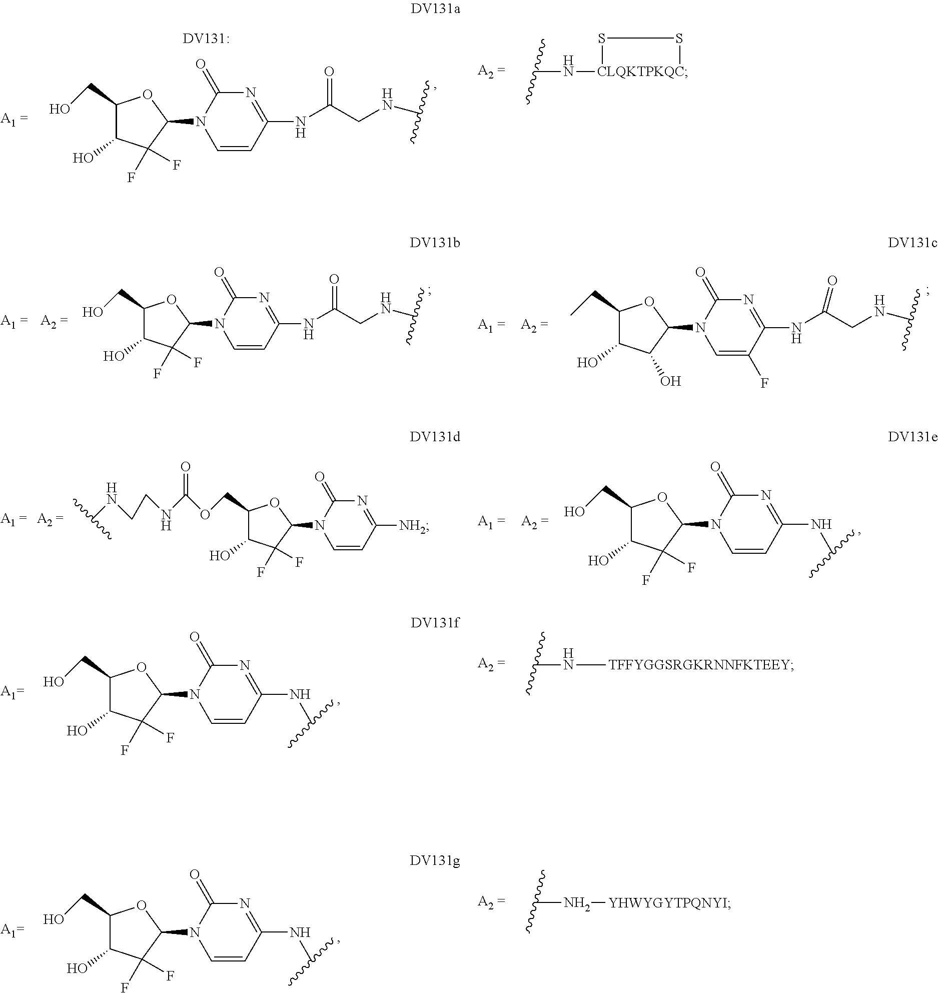

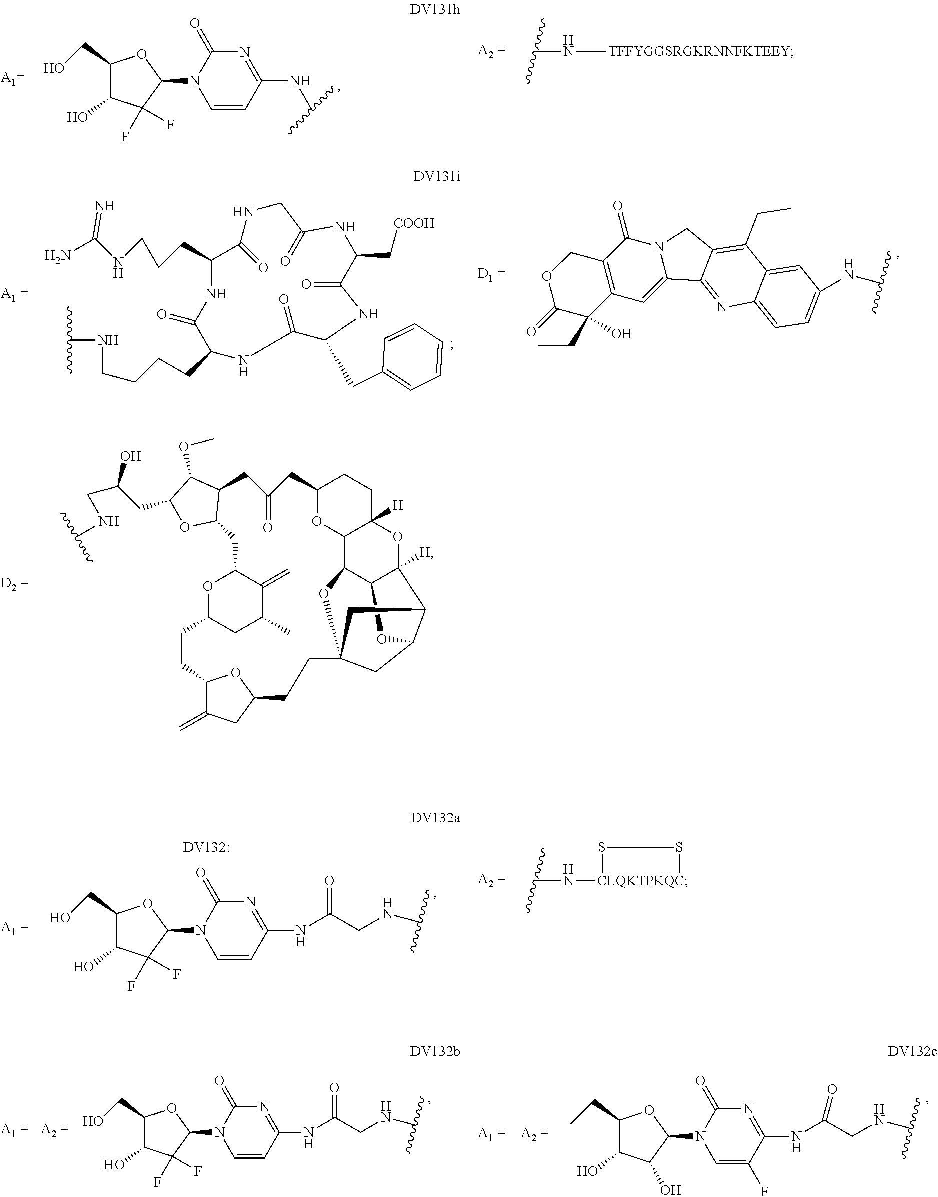

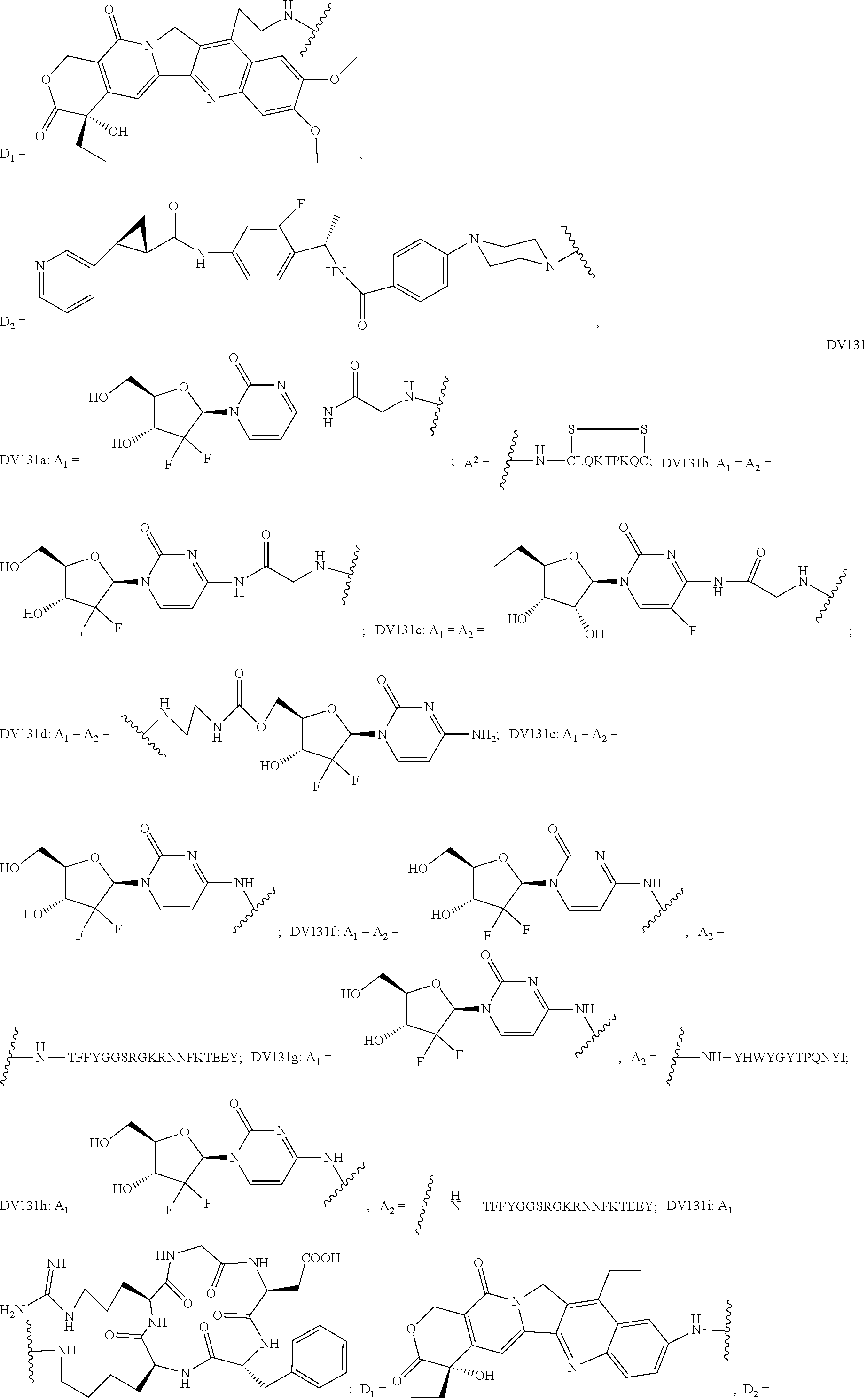

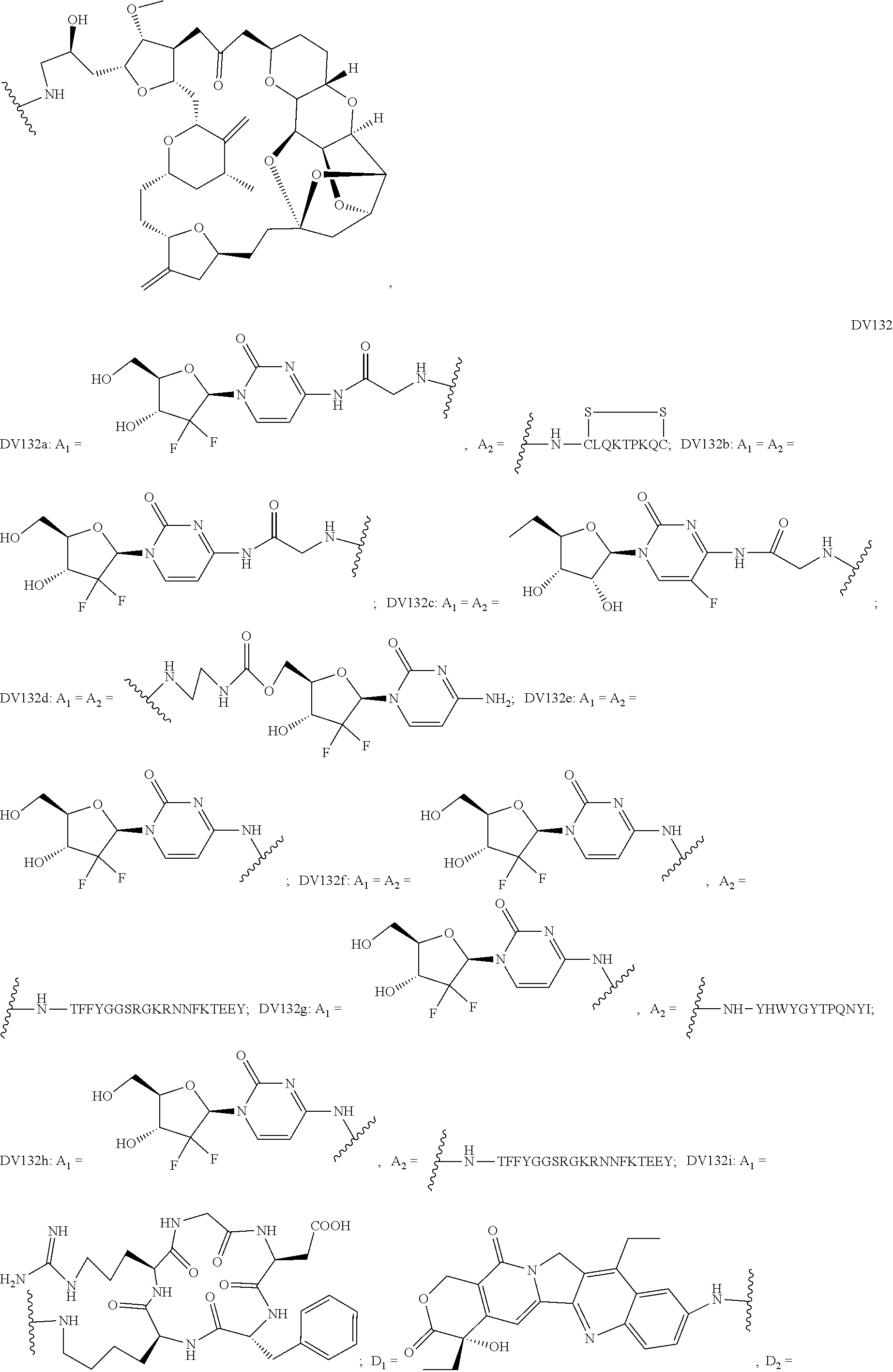

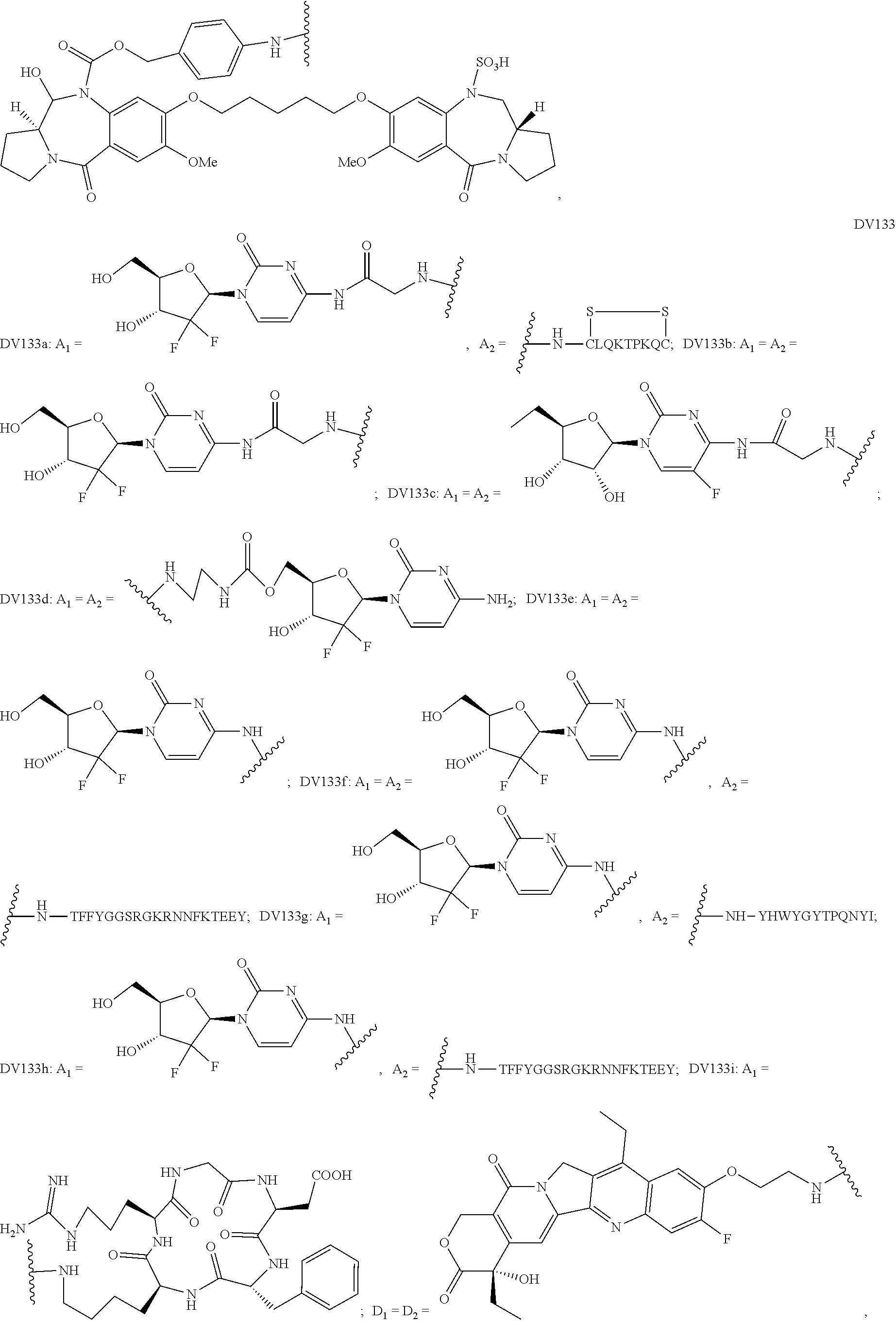

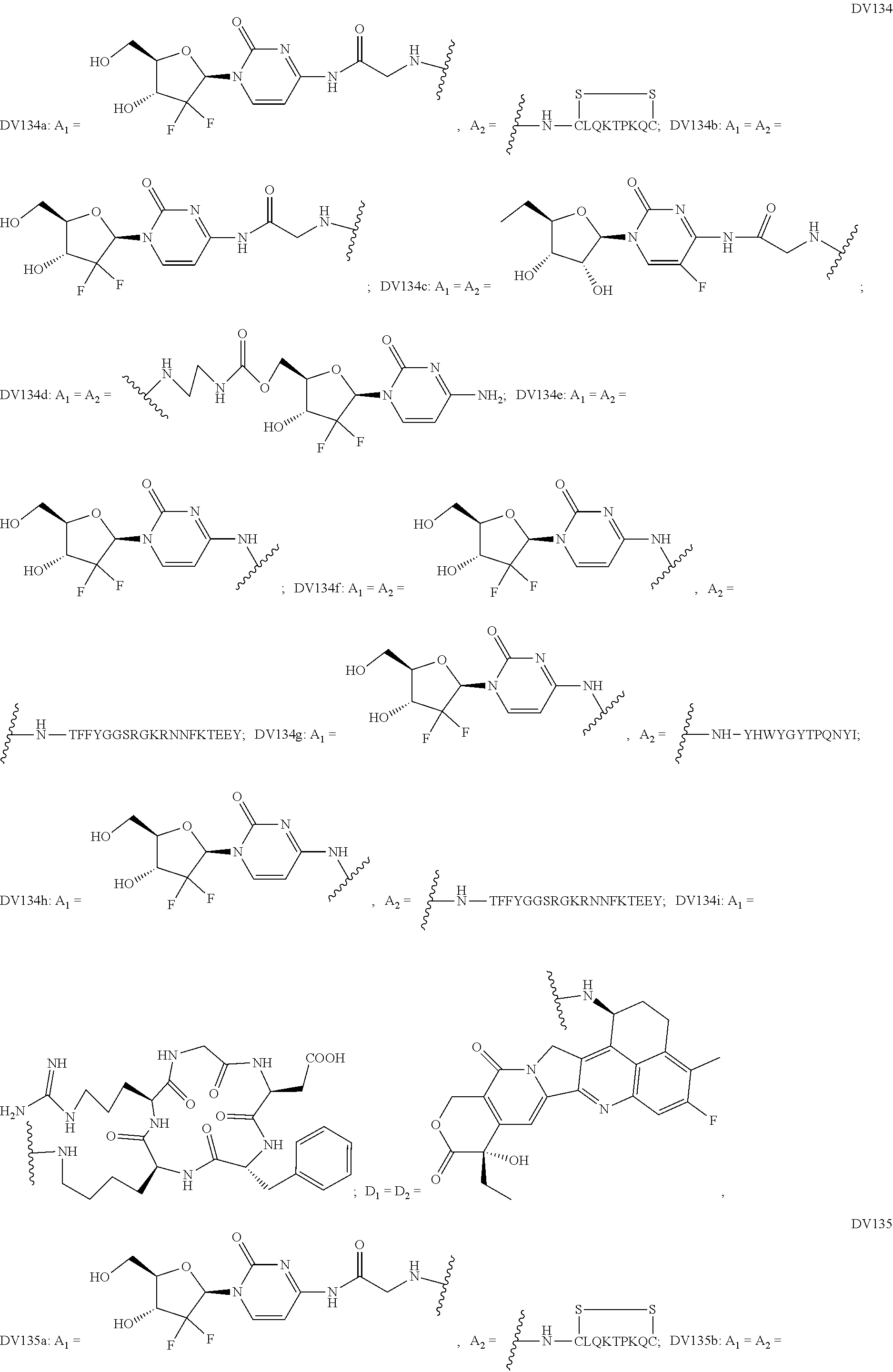

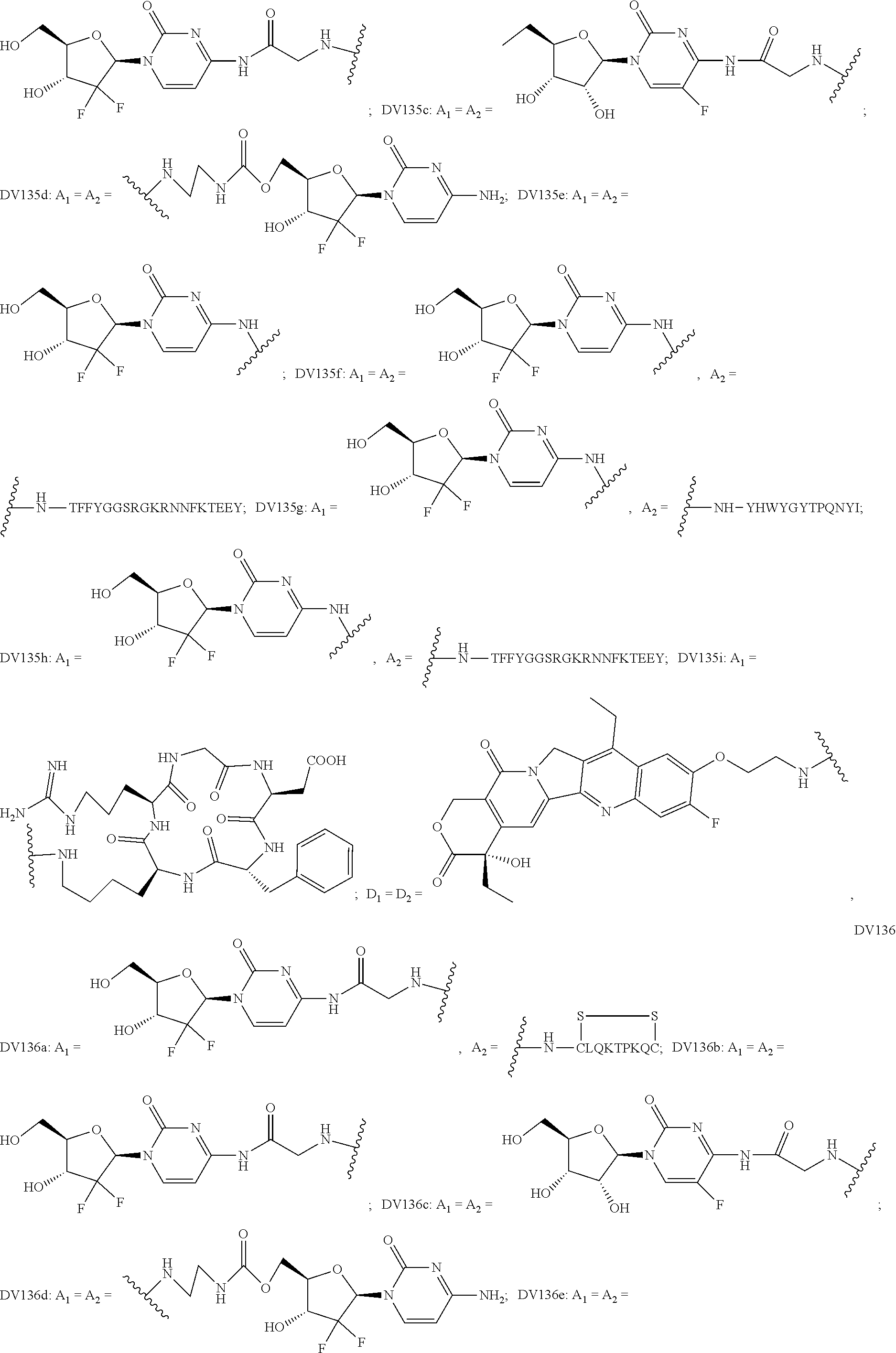

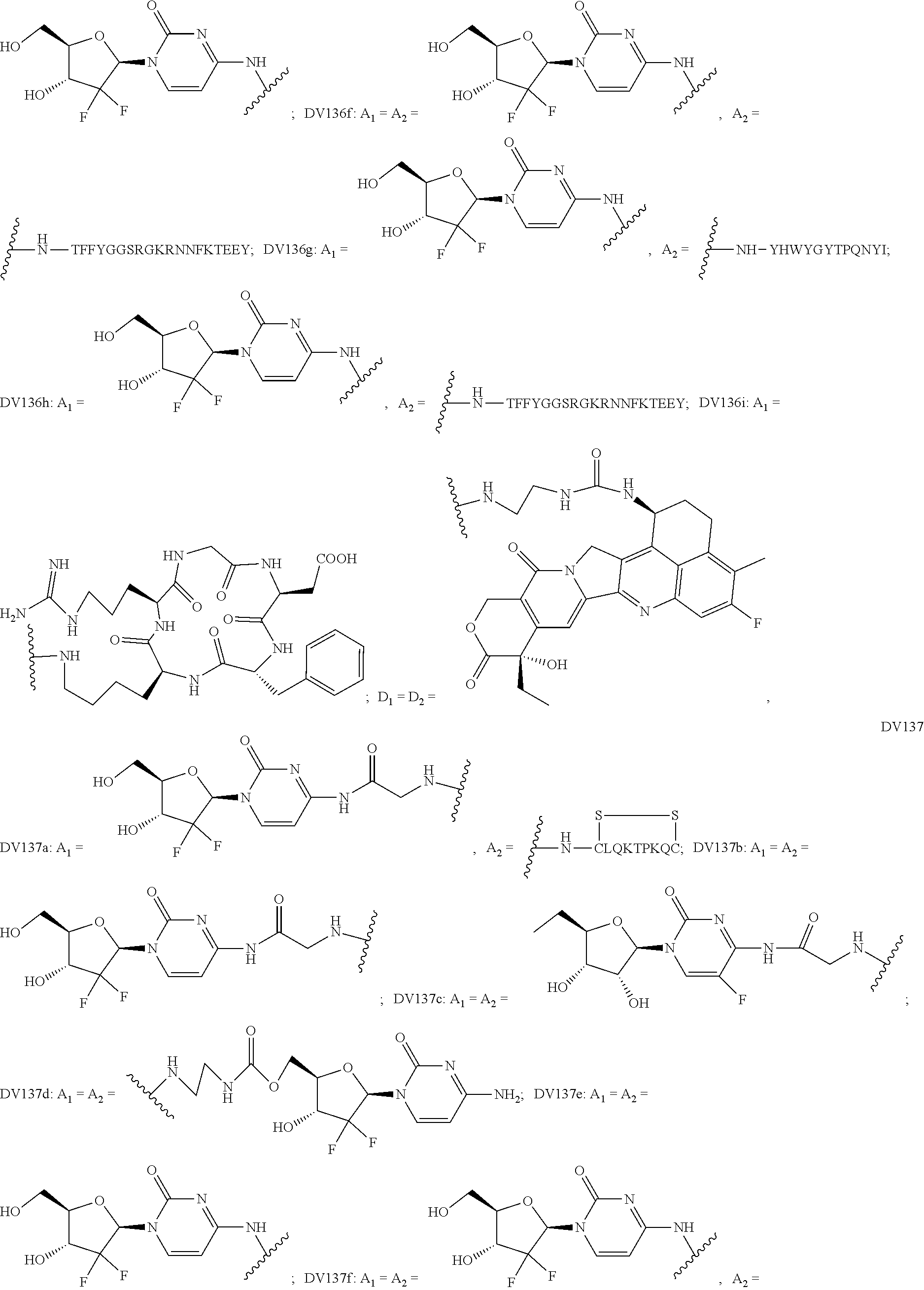

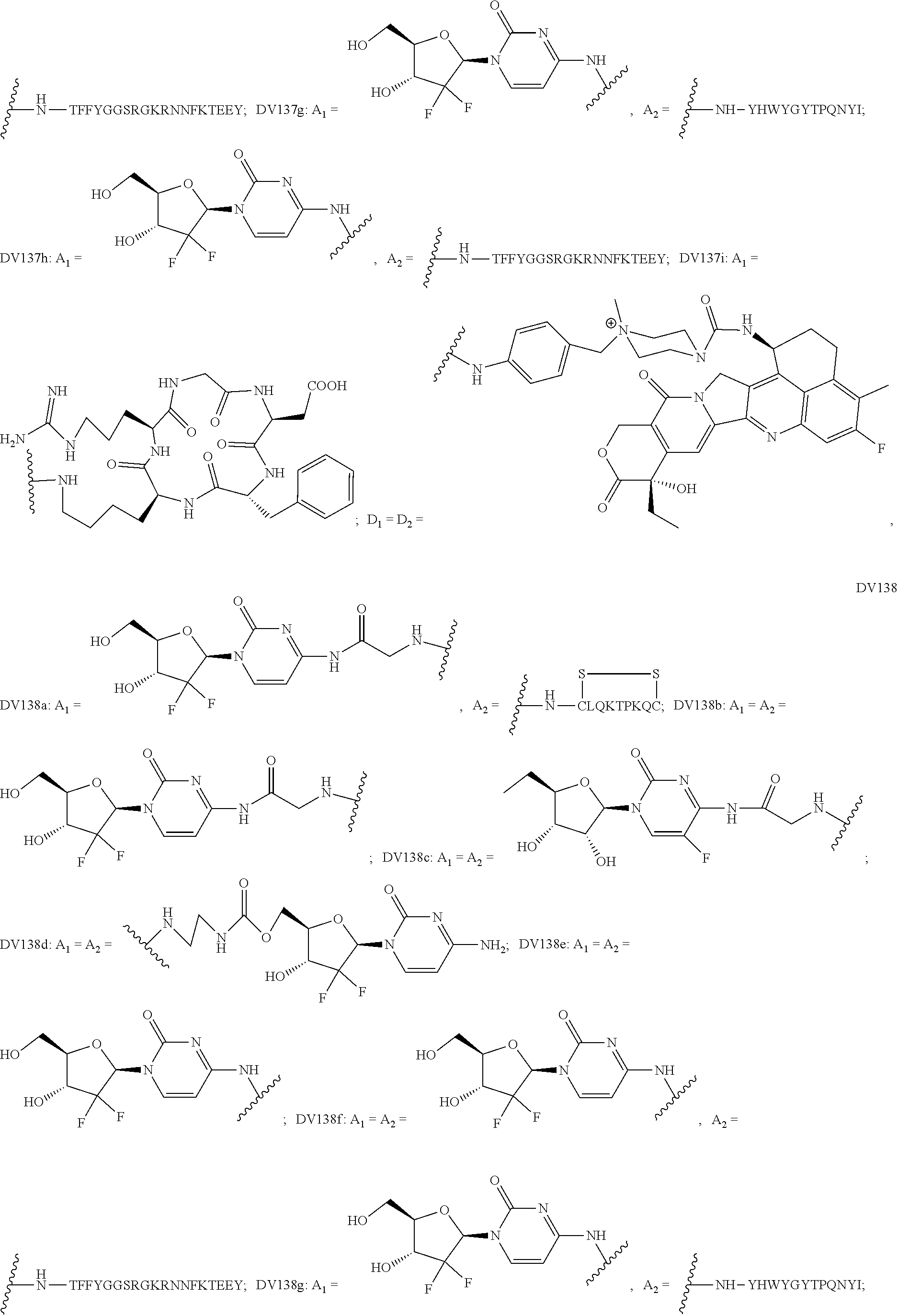

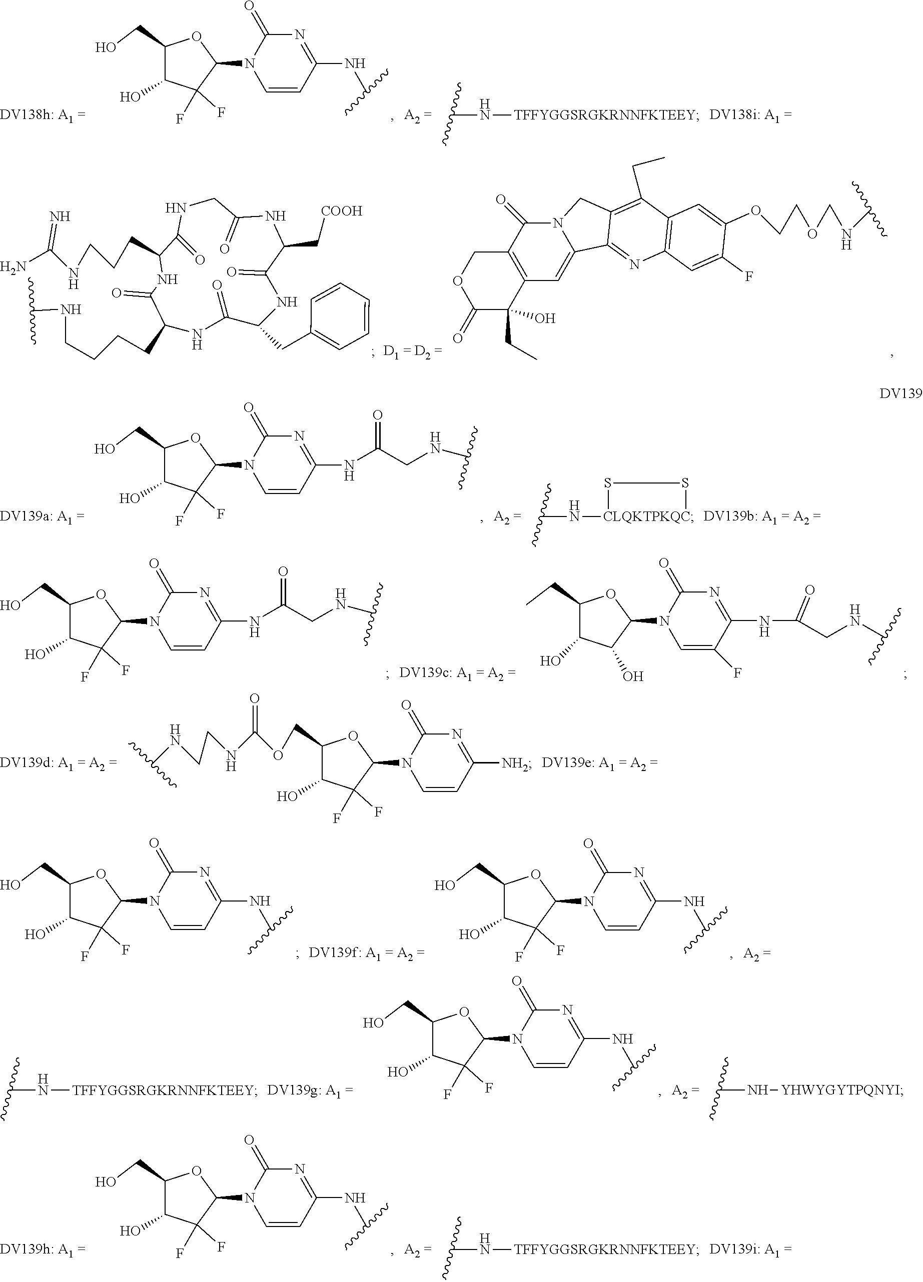

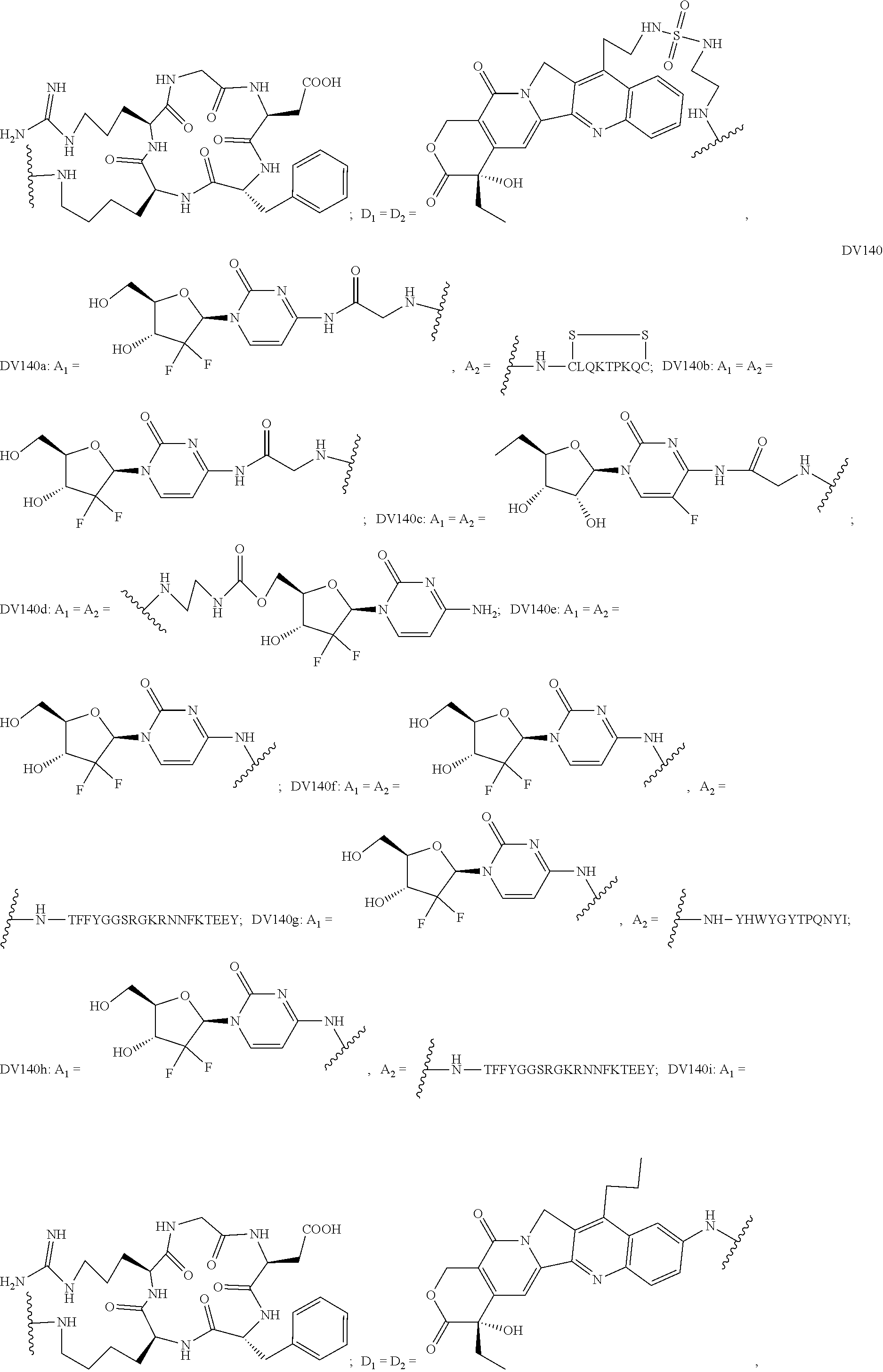

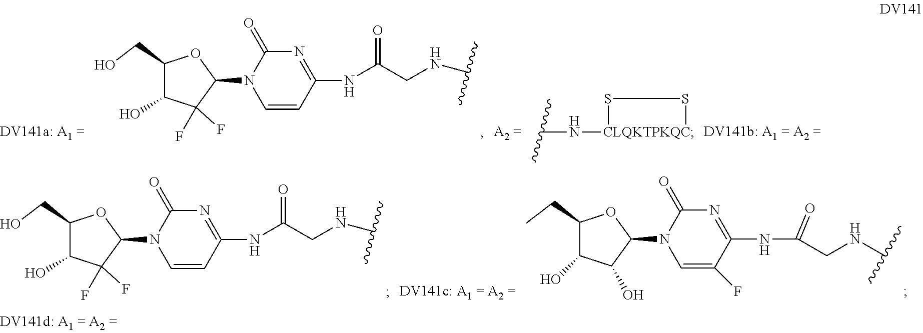

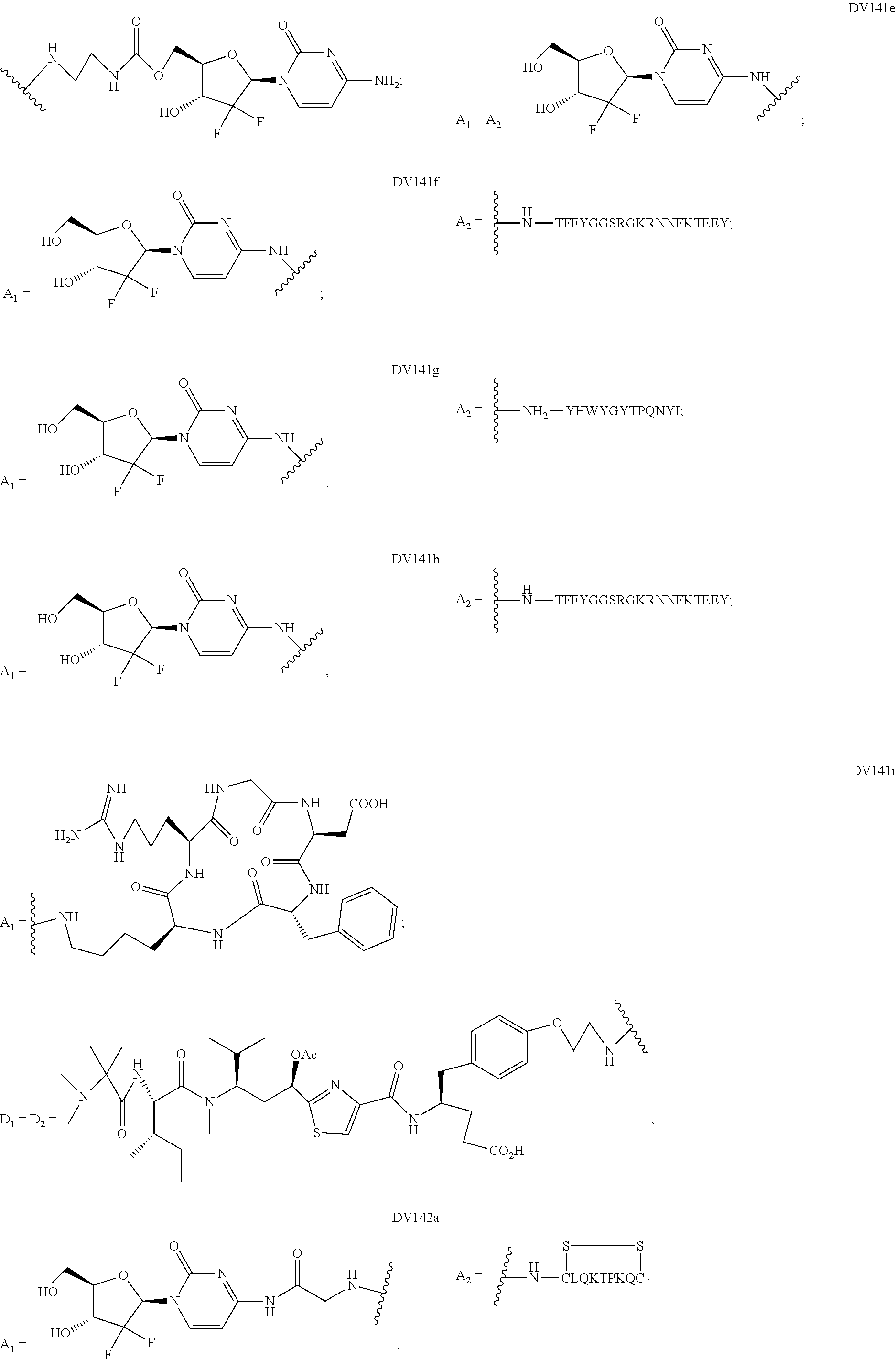

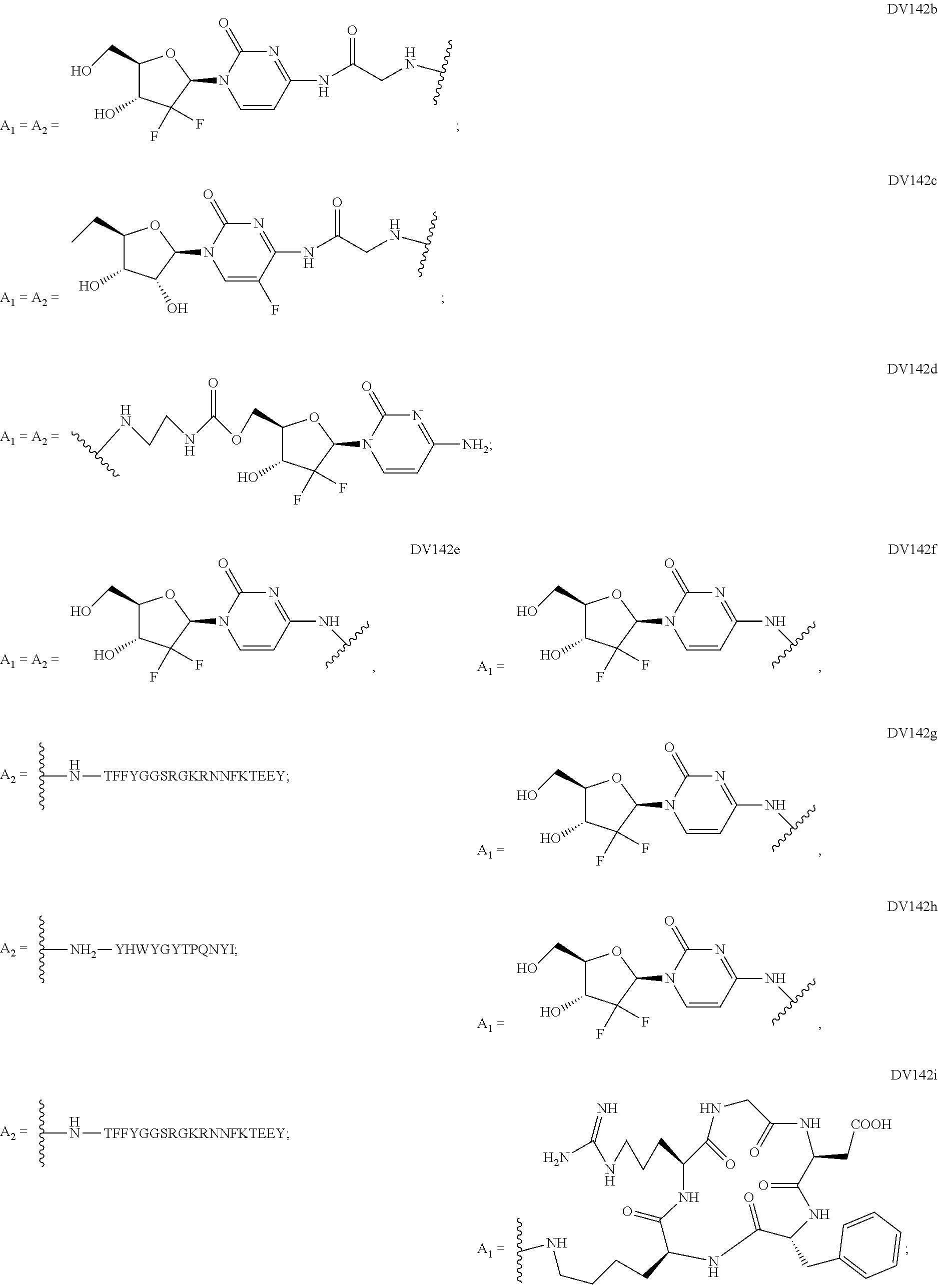

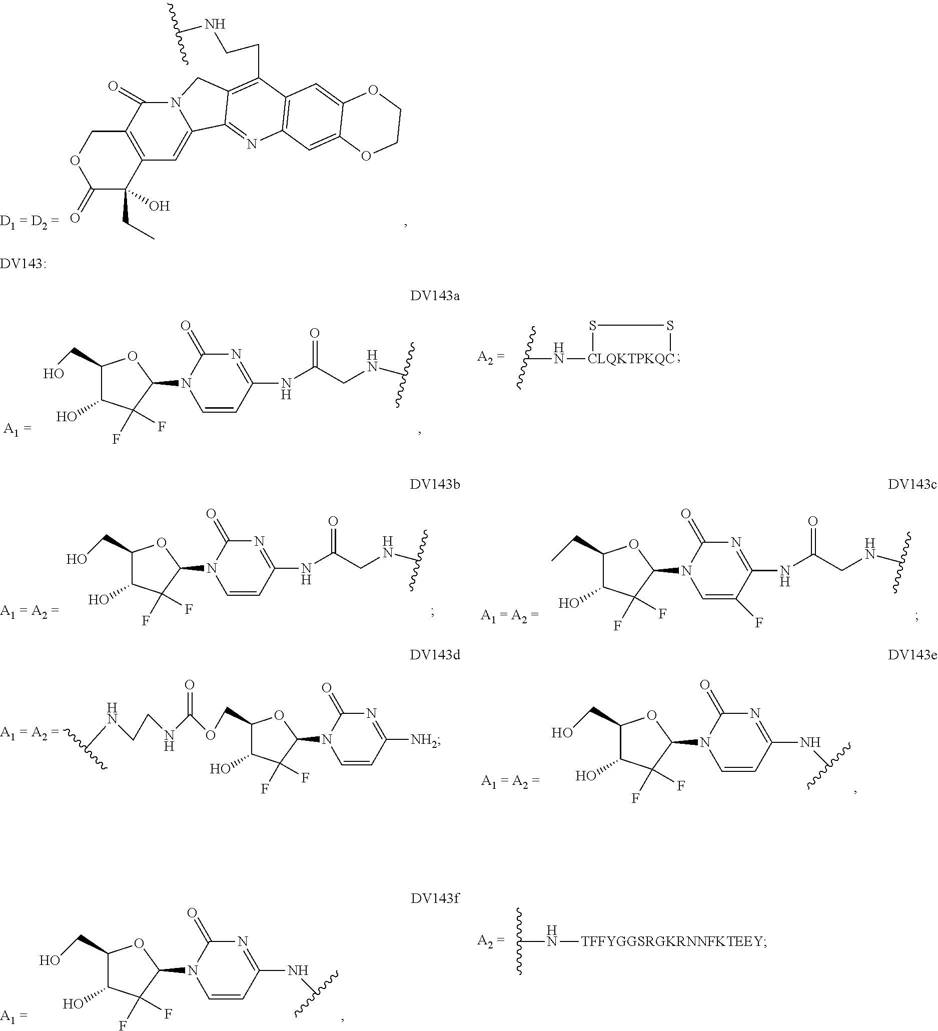

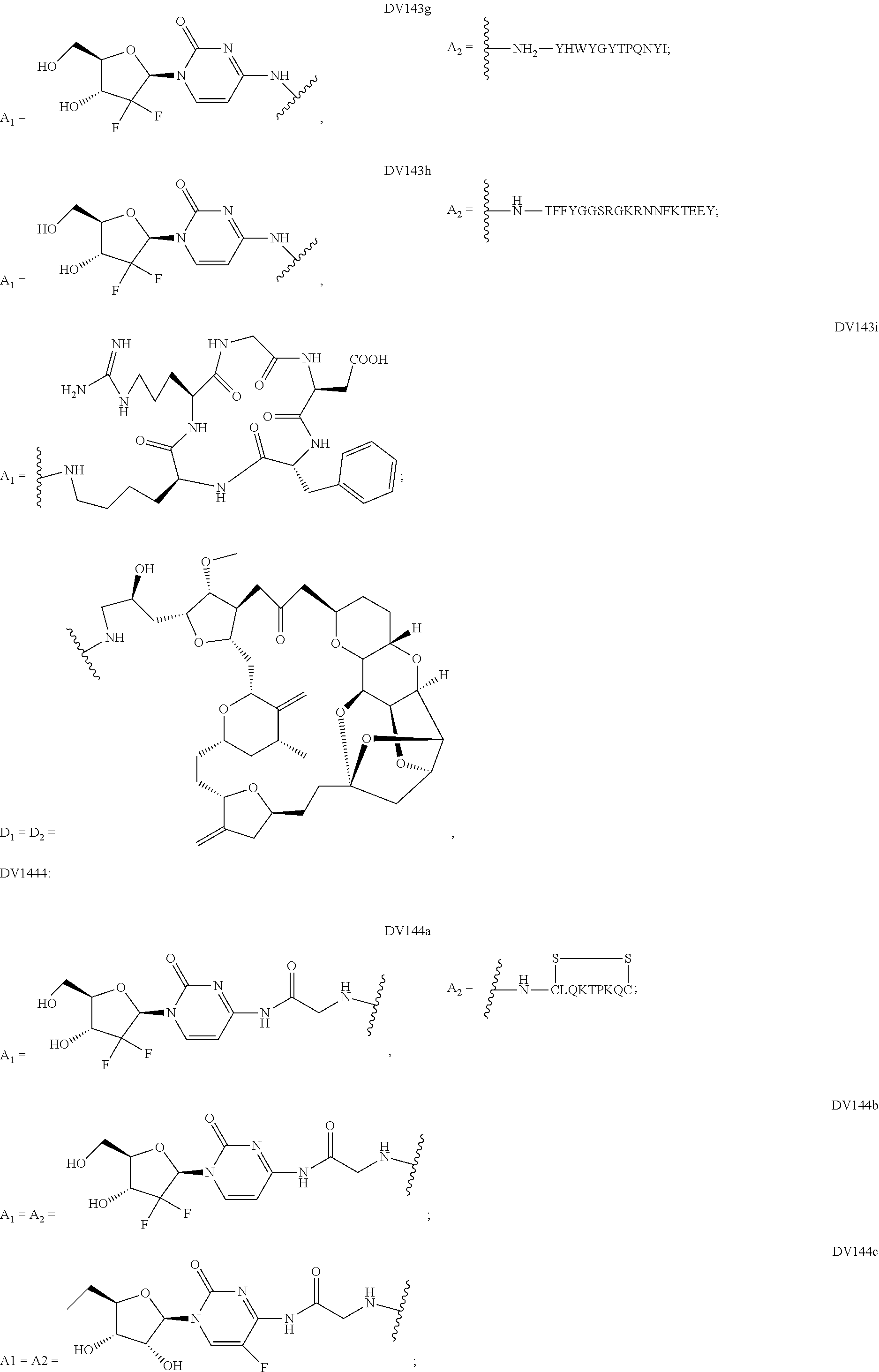

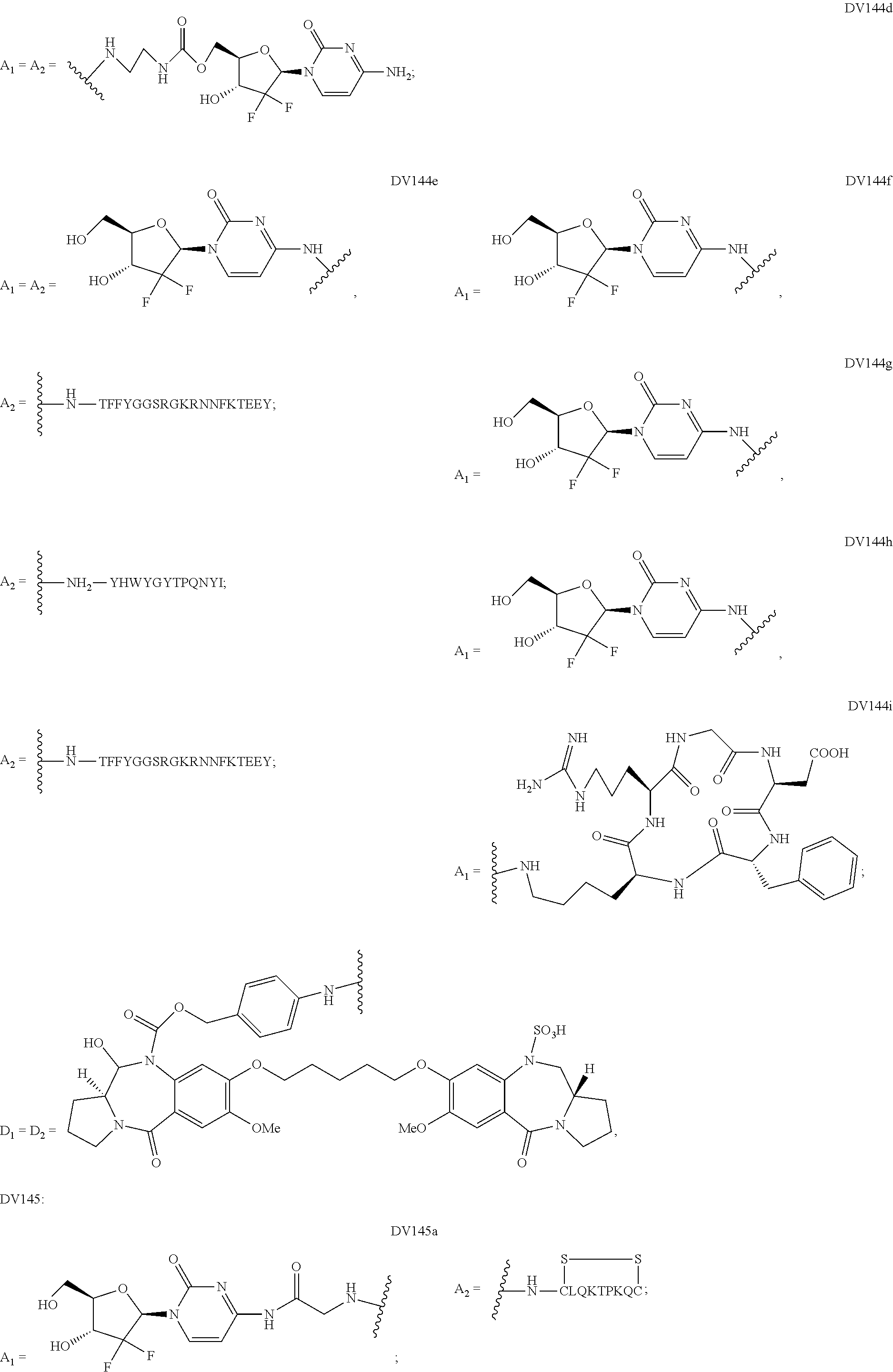

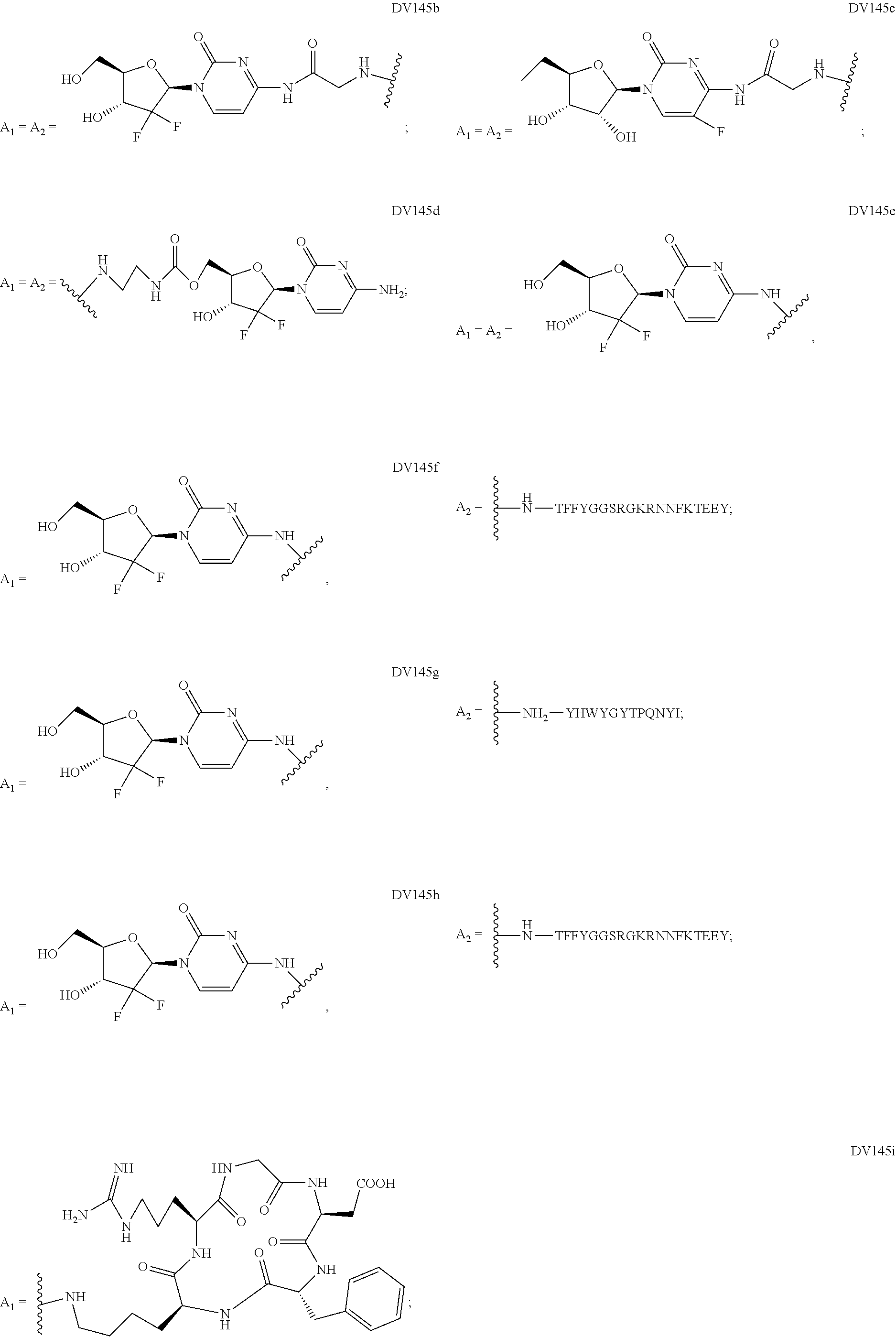

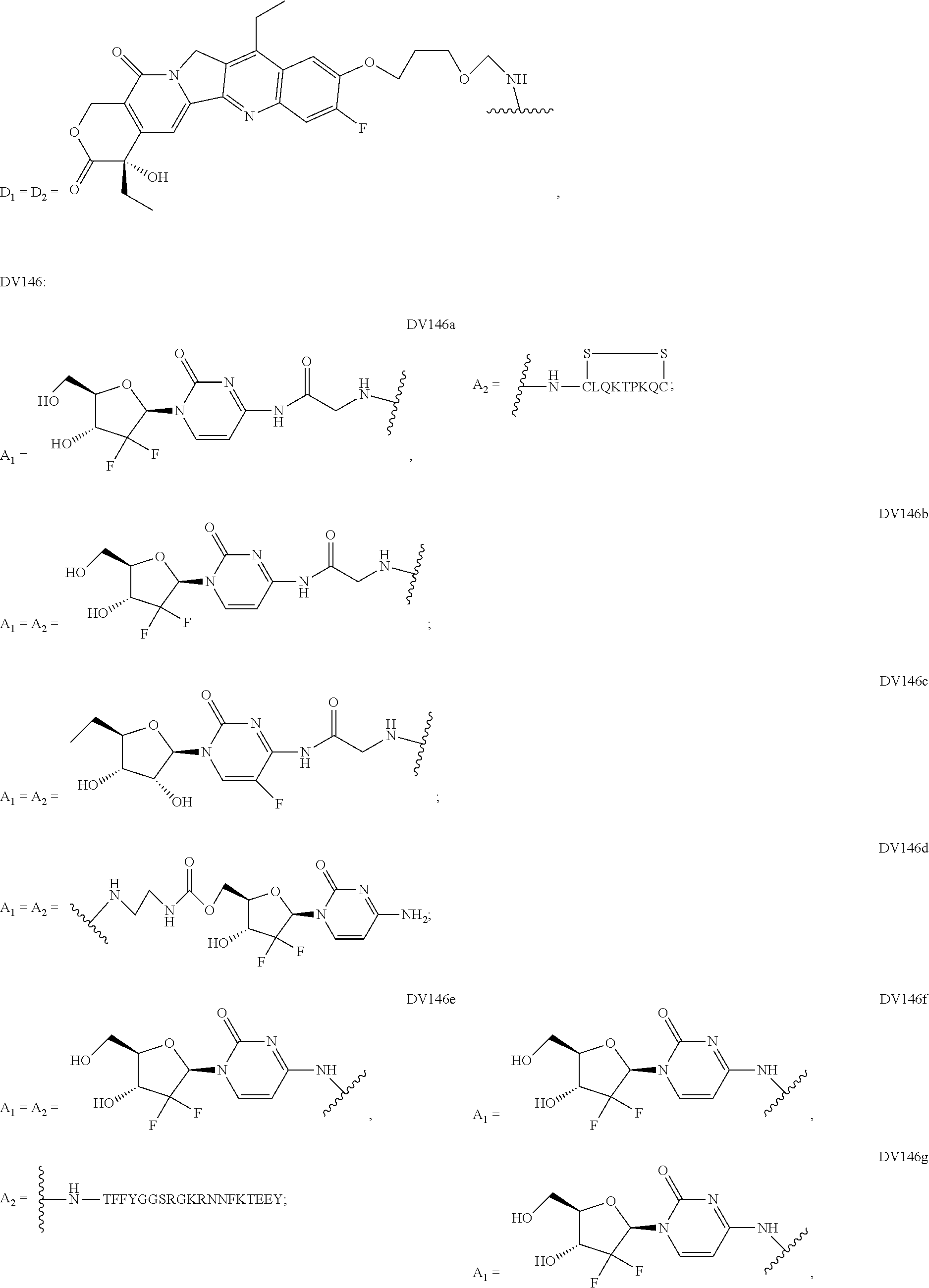

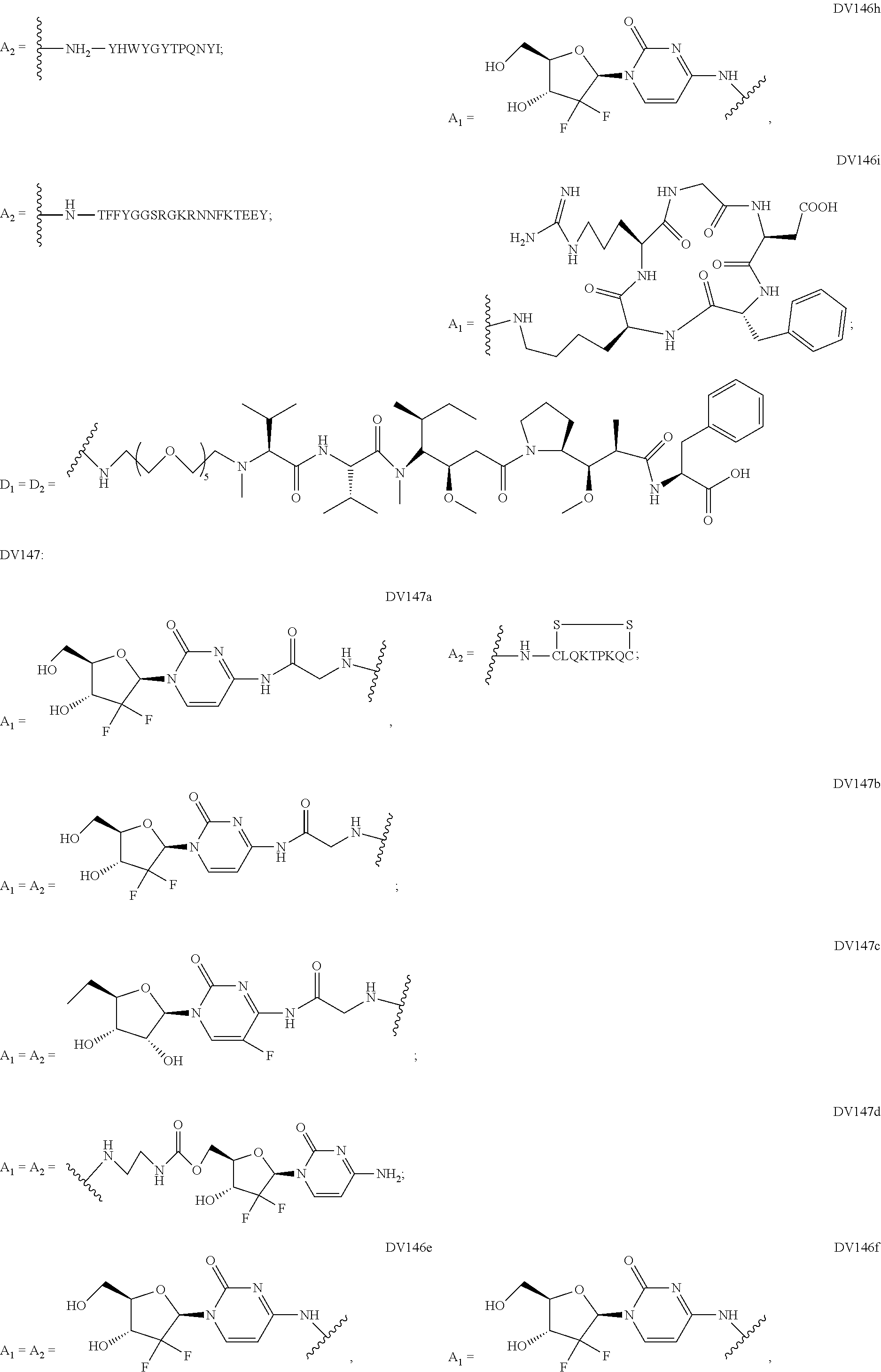

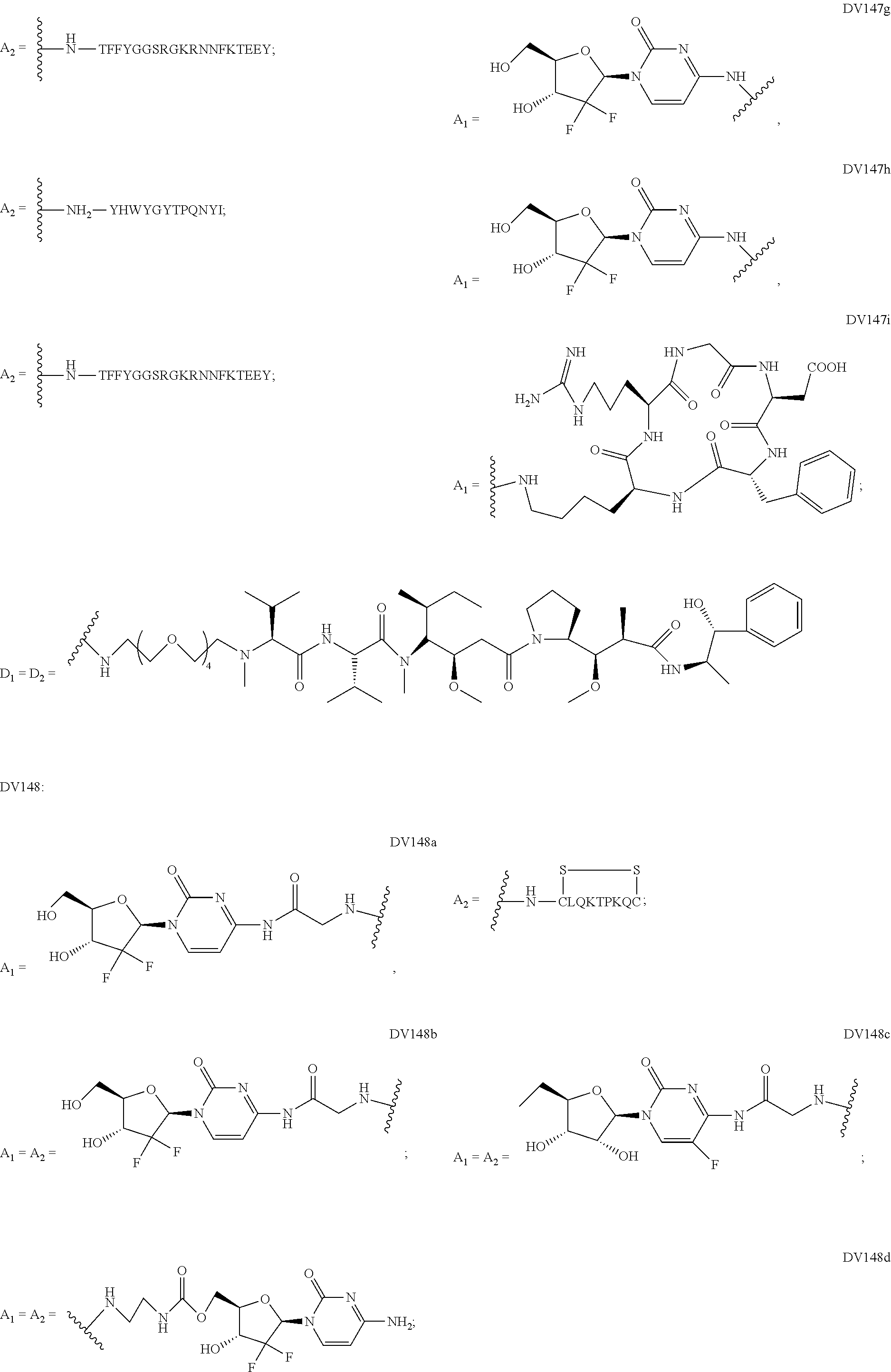

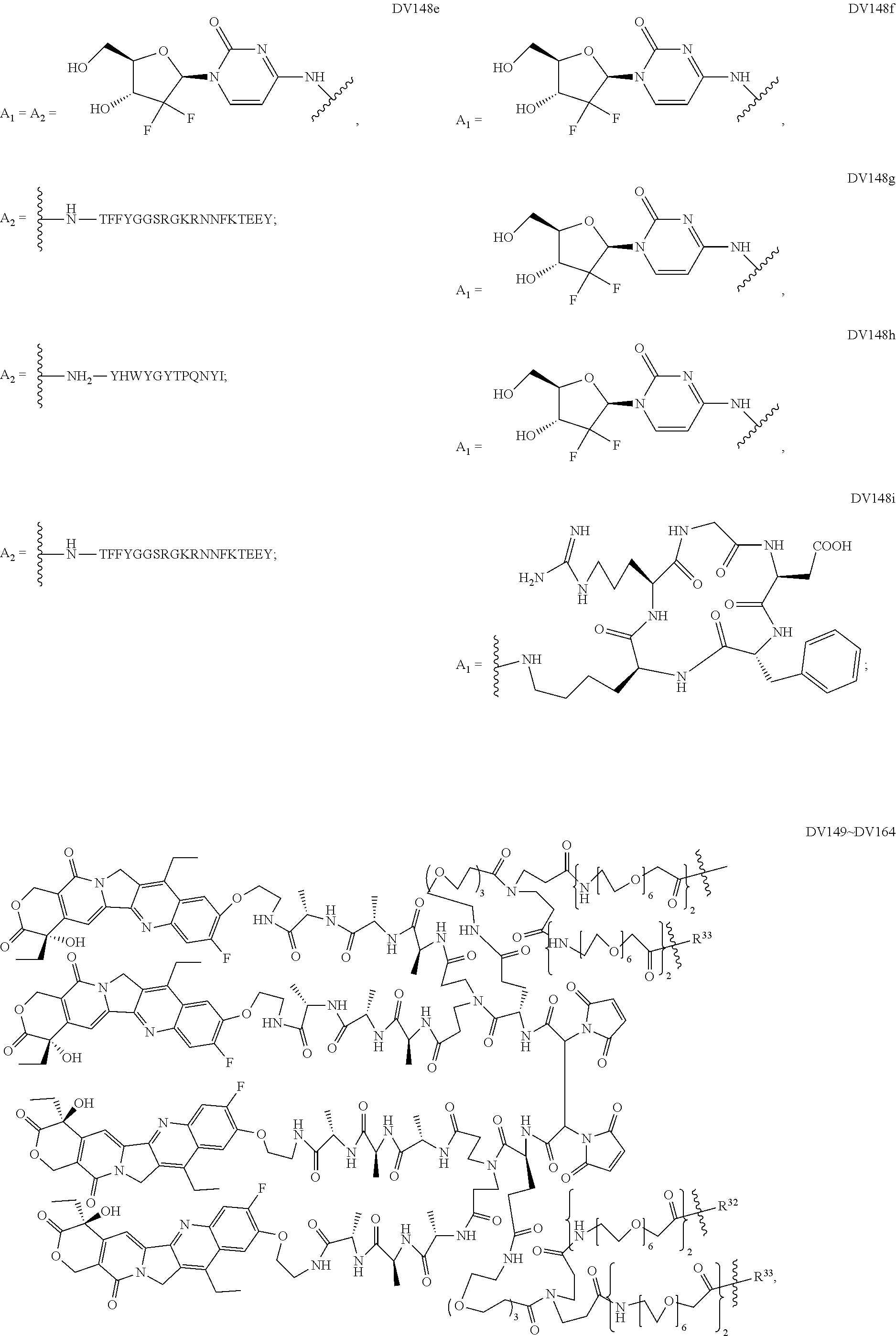

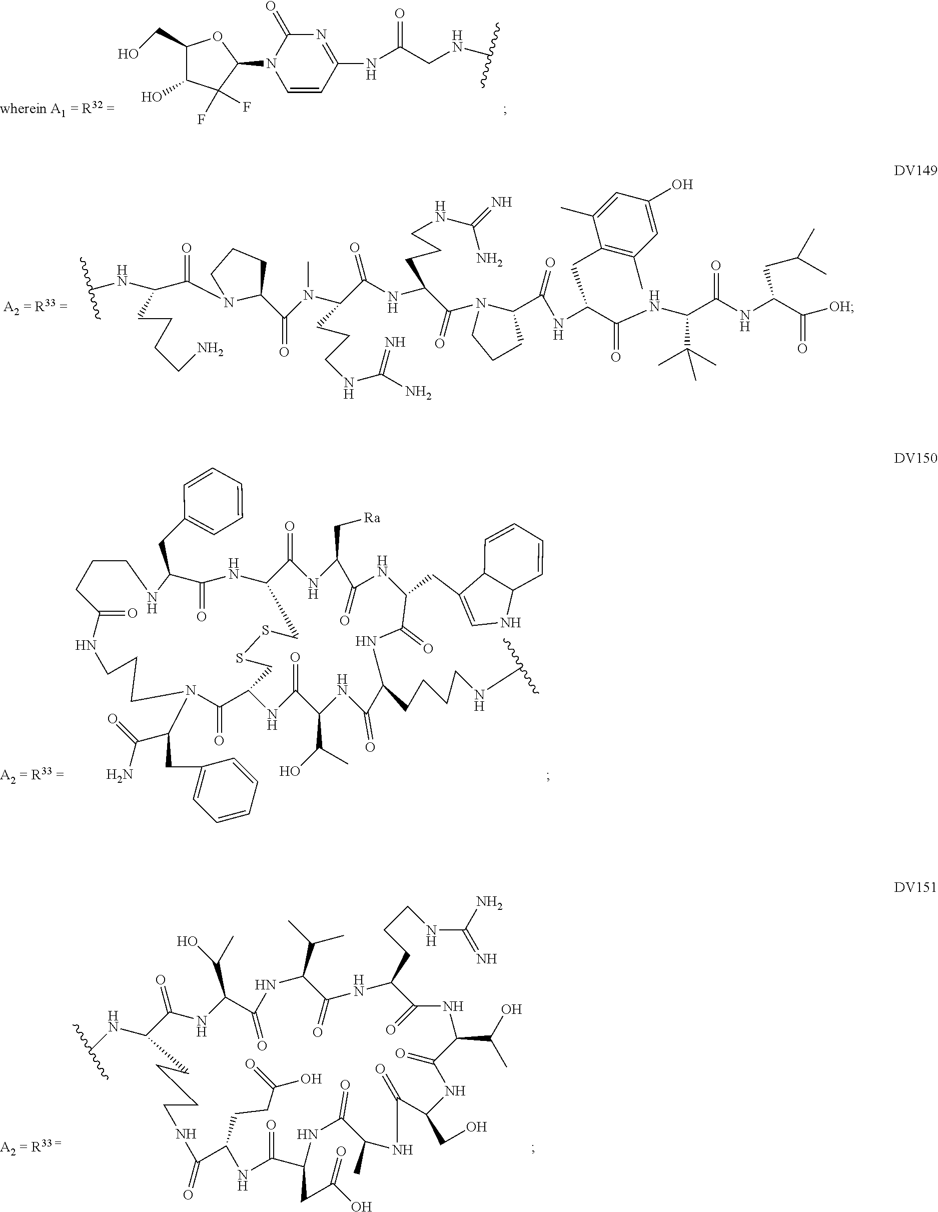

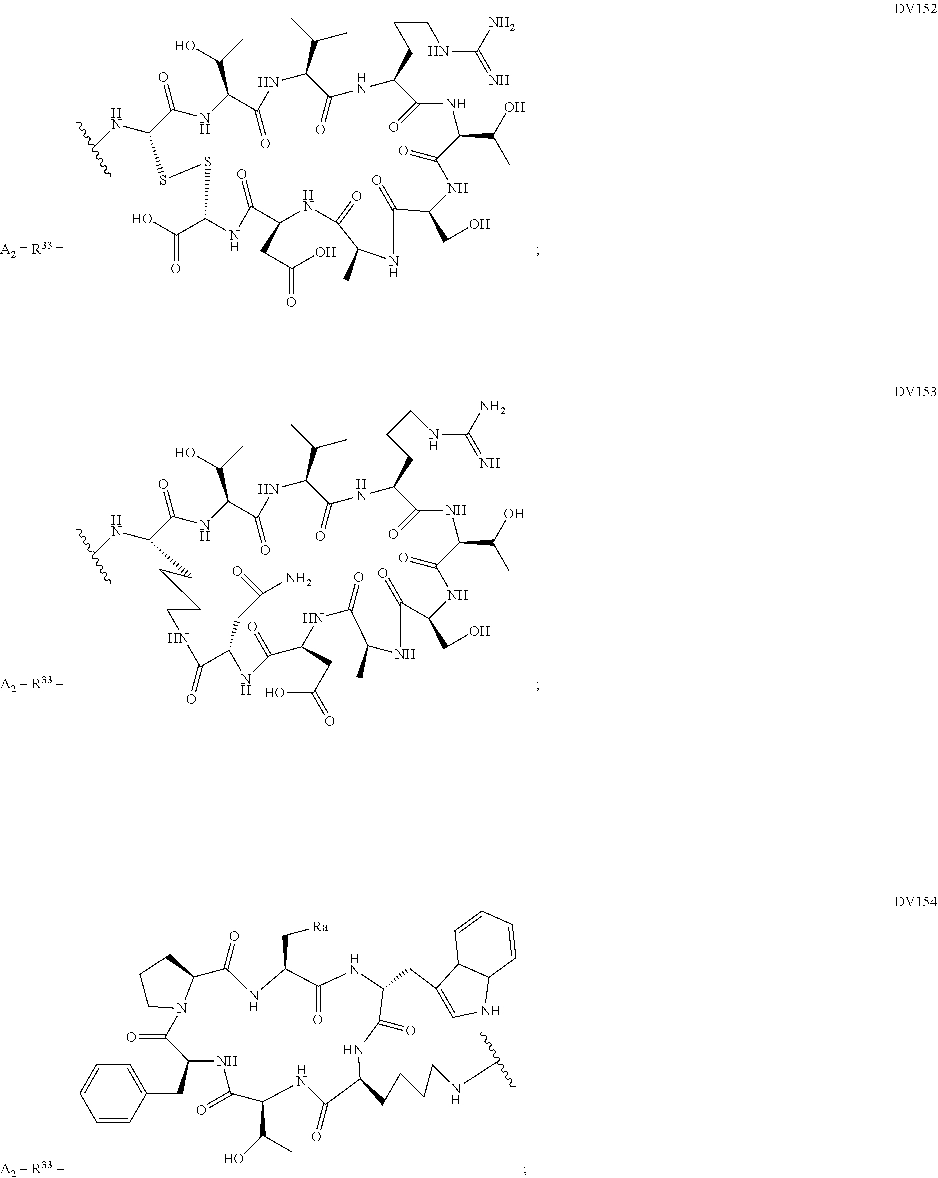

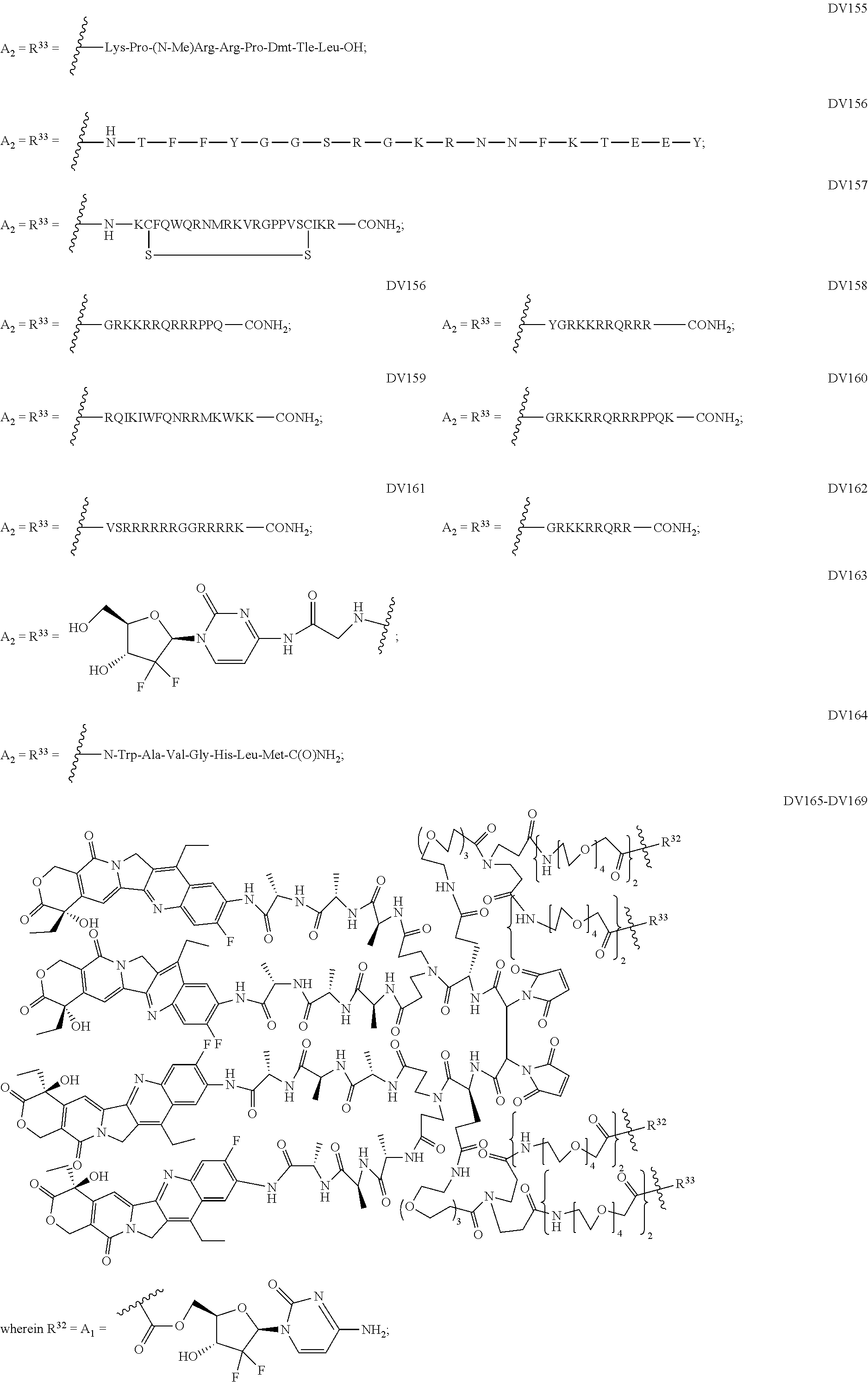

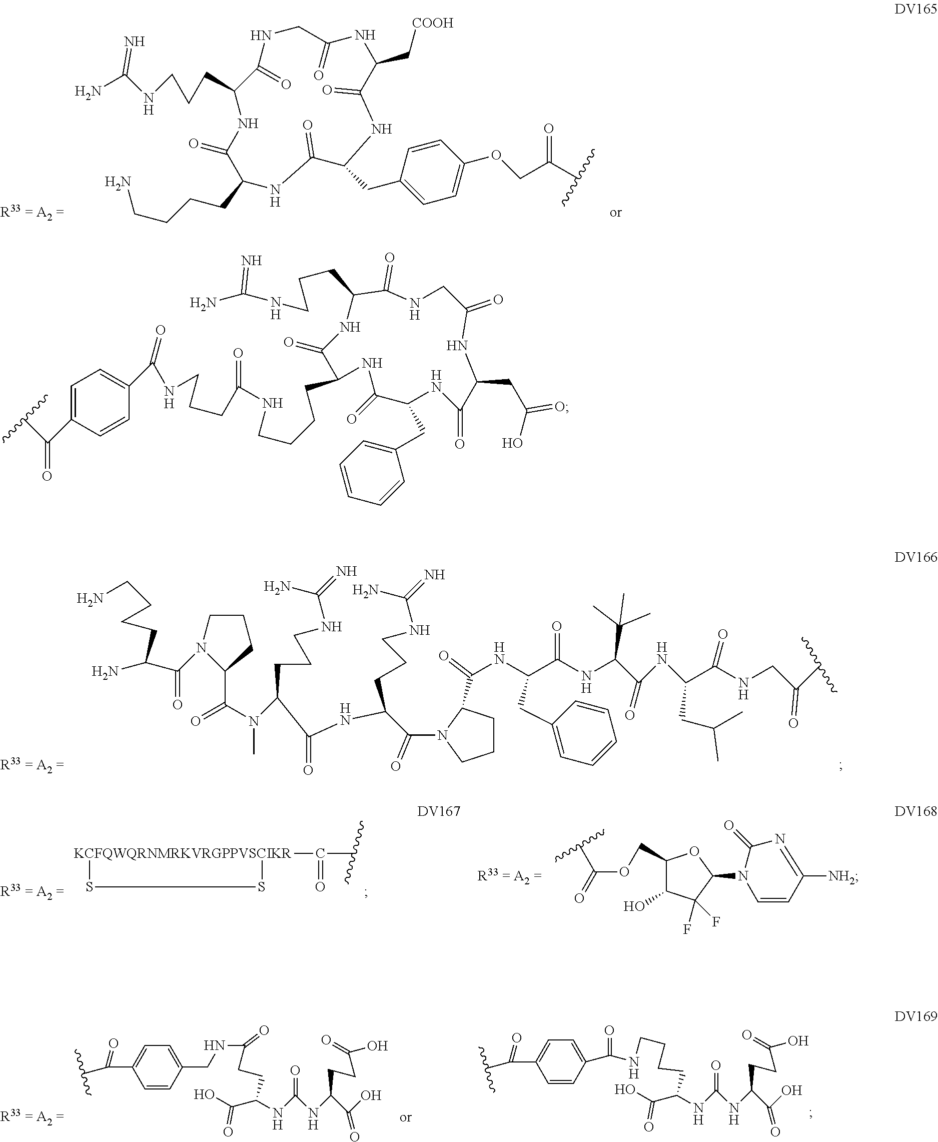

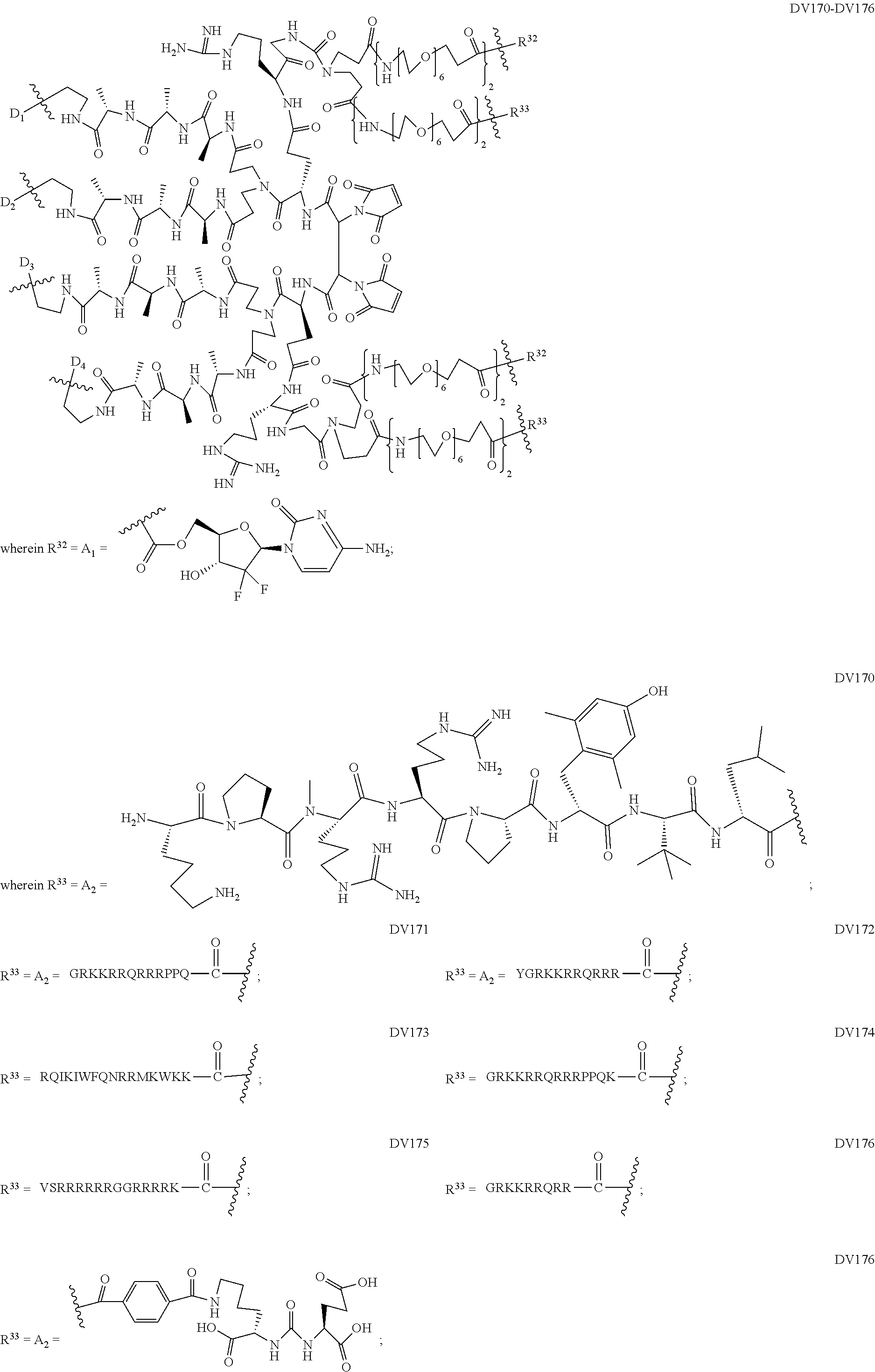

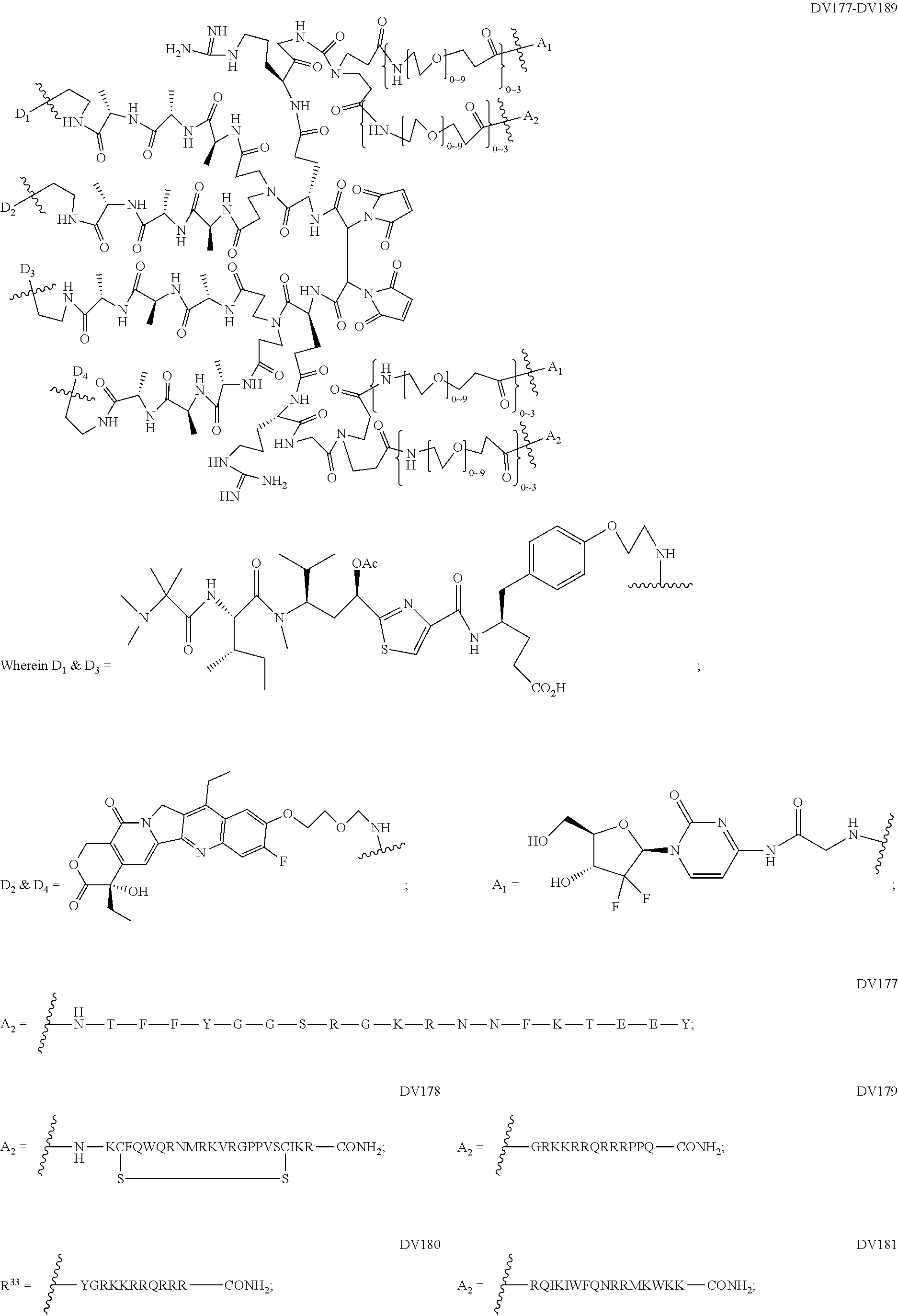

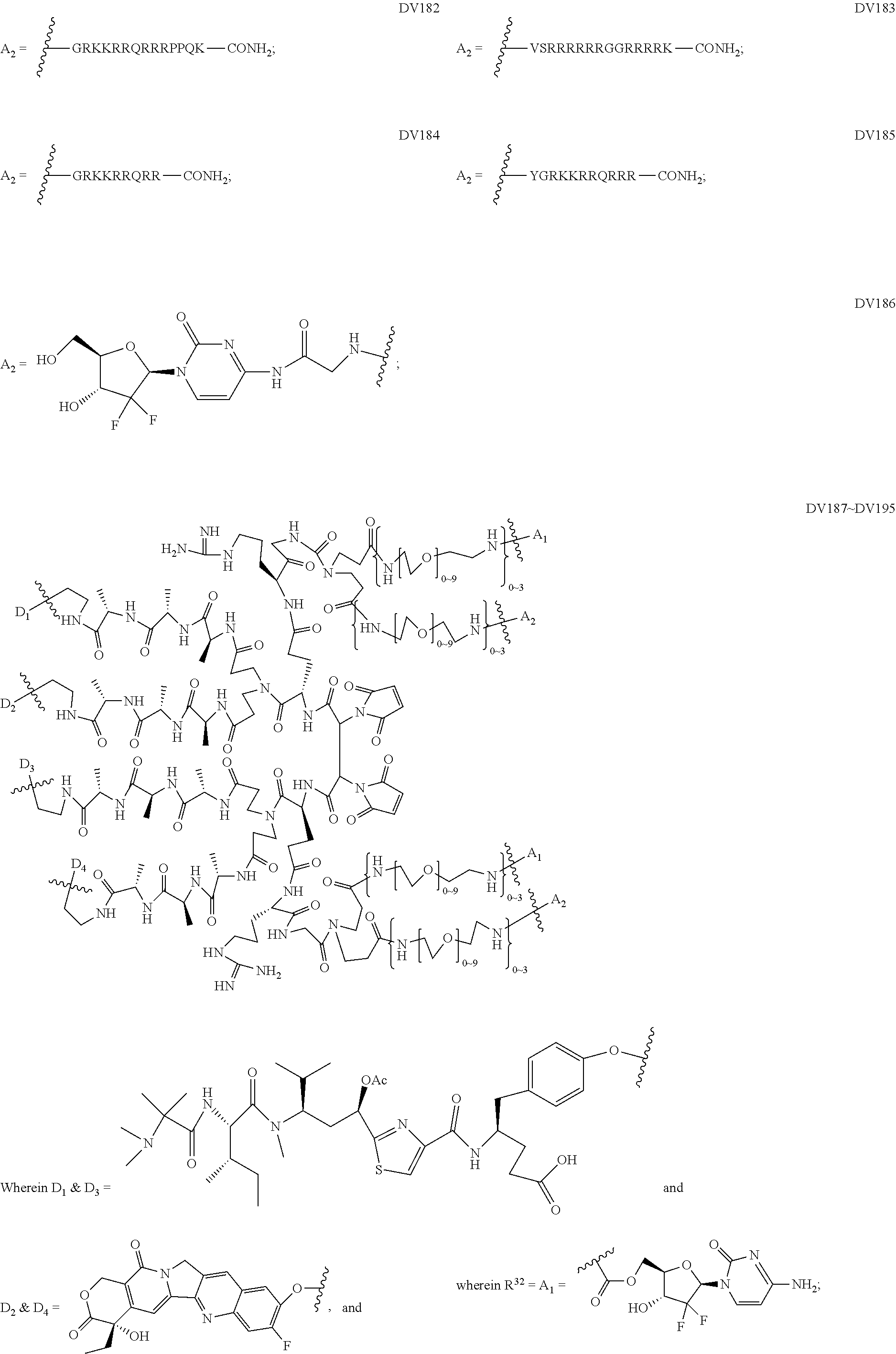

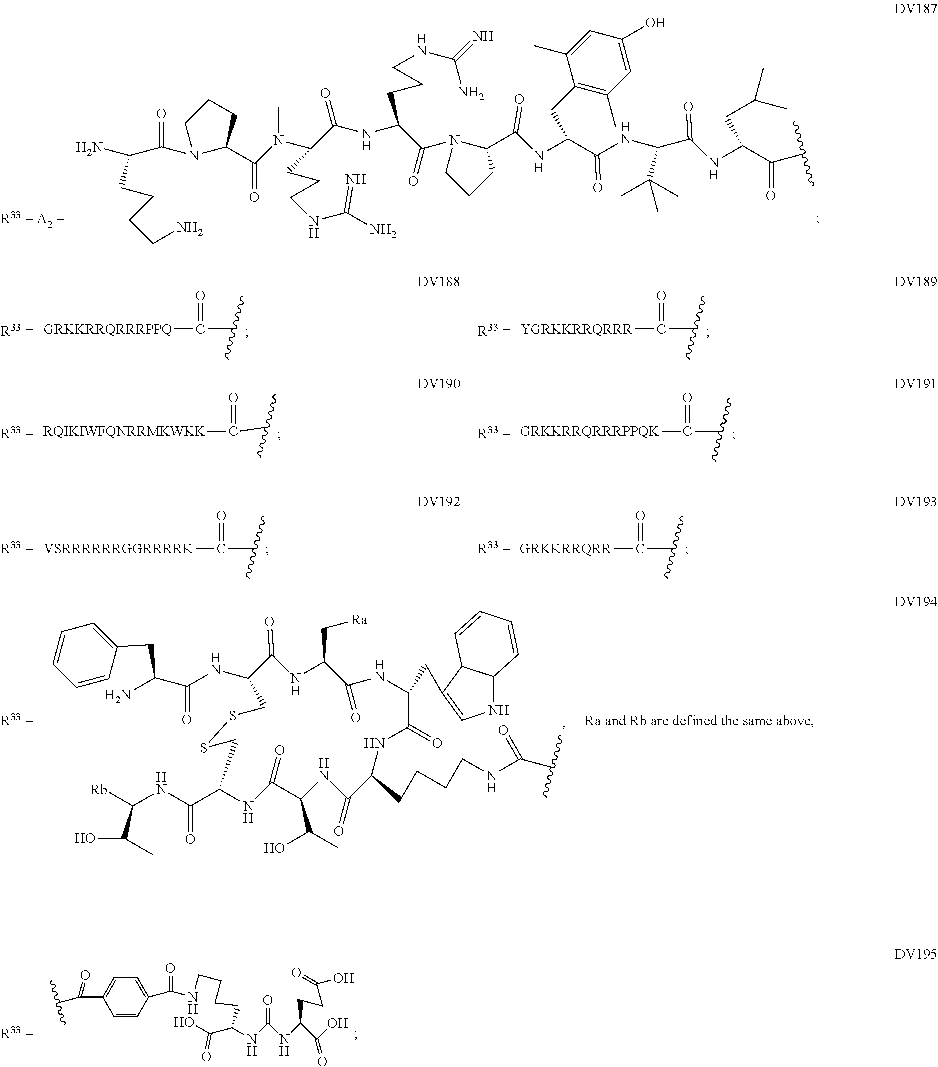

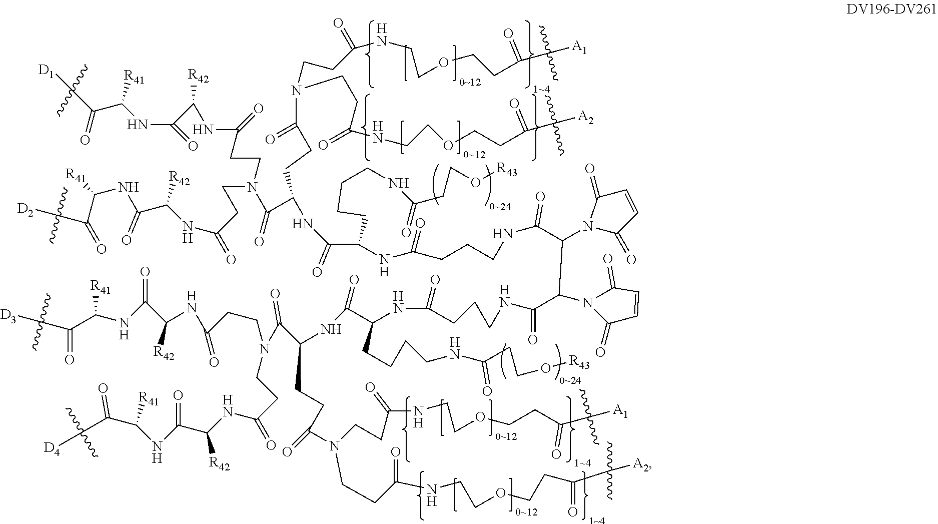

Wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H₄OH; R₄₃ is H, CH₃, CH₂CH₃, C(O)H, and C(O)CH₃; D1, D2, D3 and D4 are cytotoxic drug described in this application, and A₁ and A₂ are small molecular ligands or drugs described in this application.

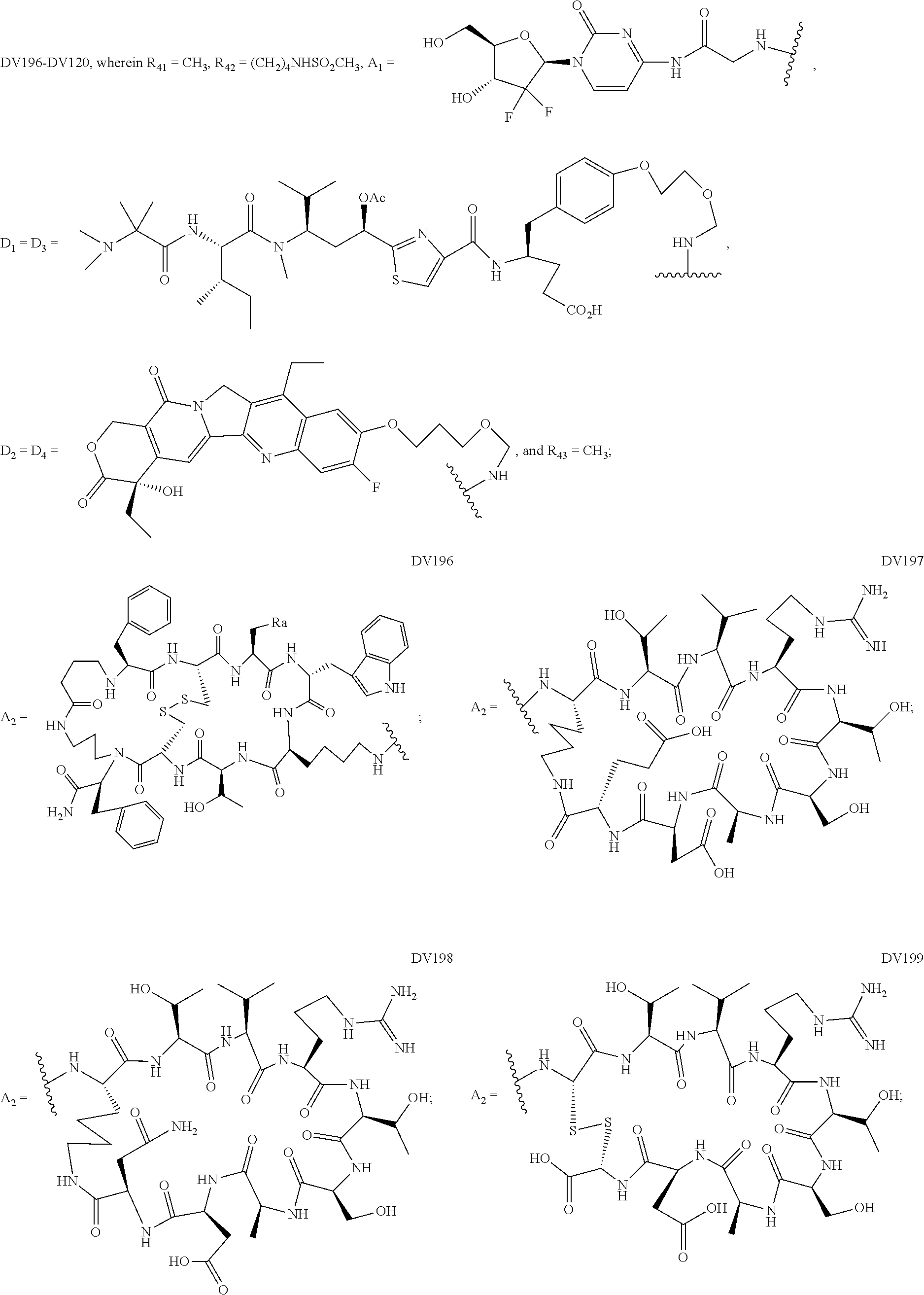

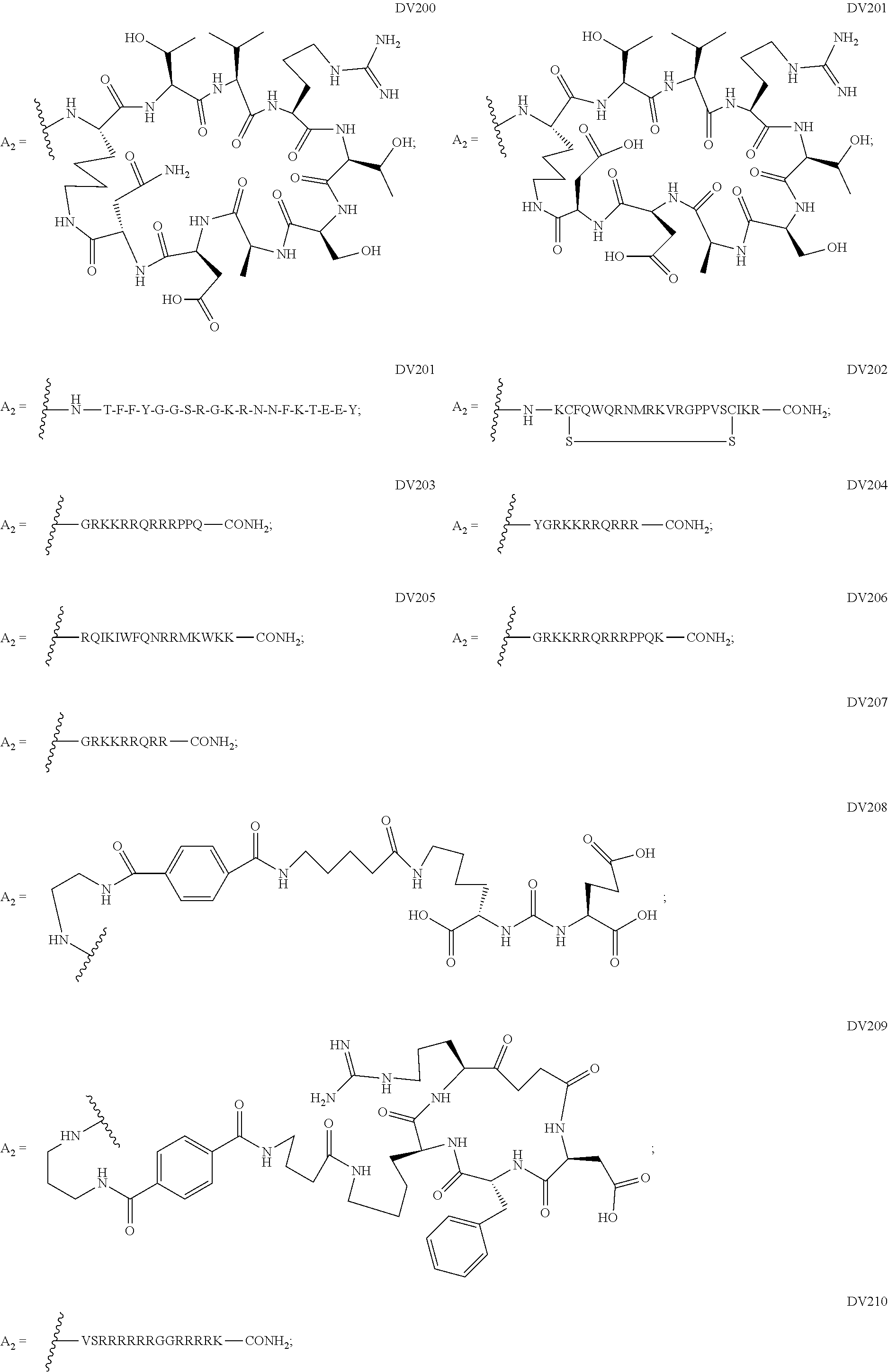

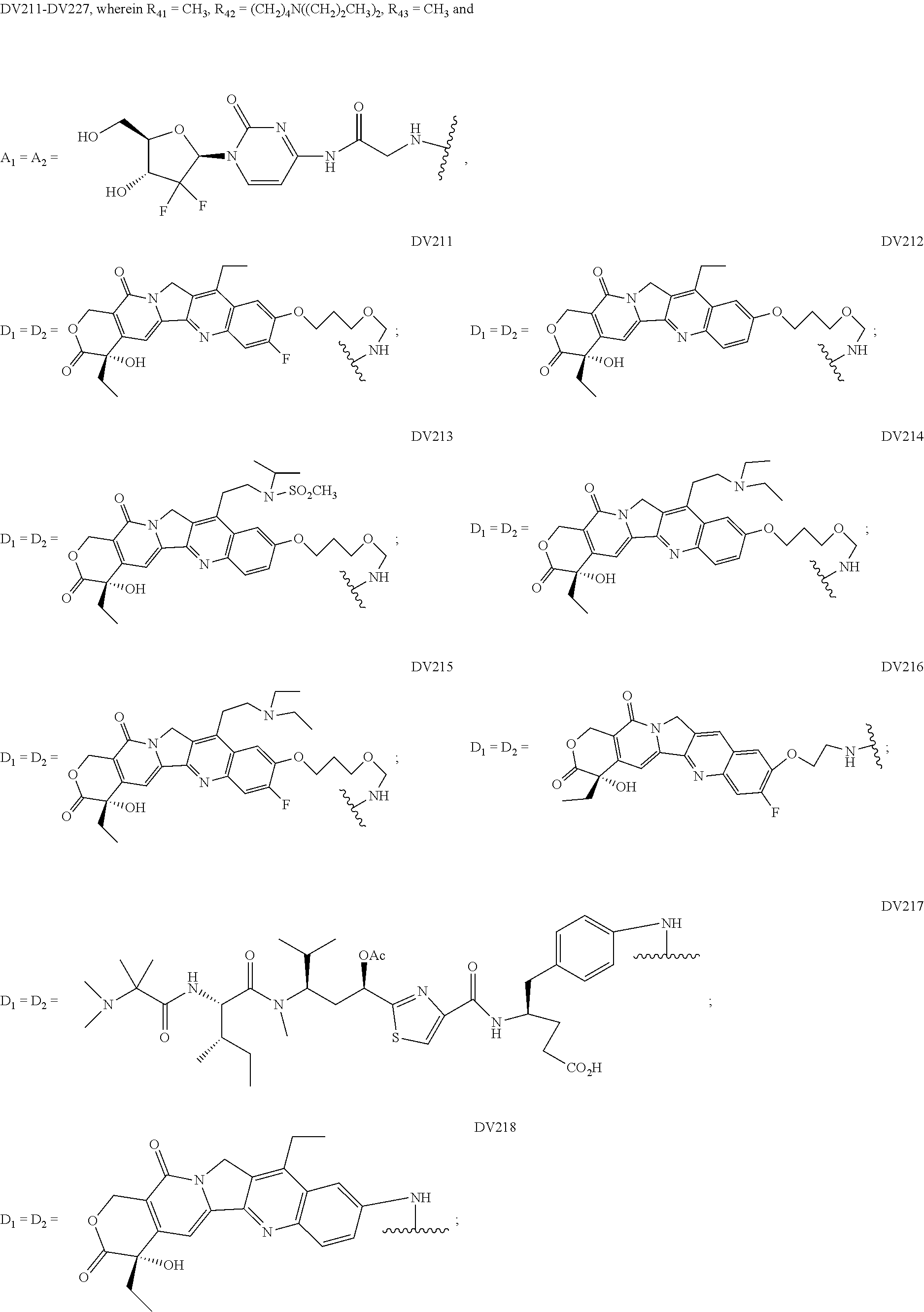

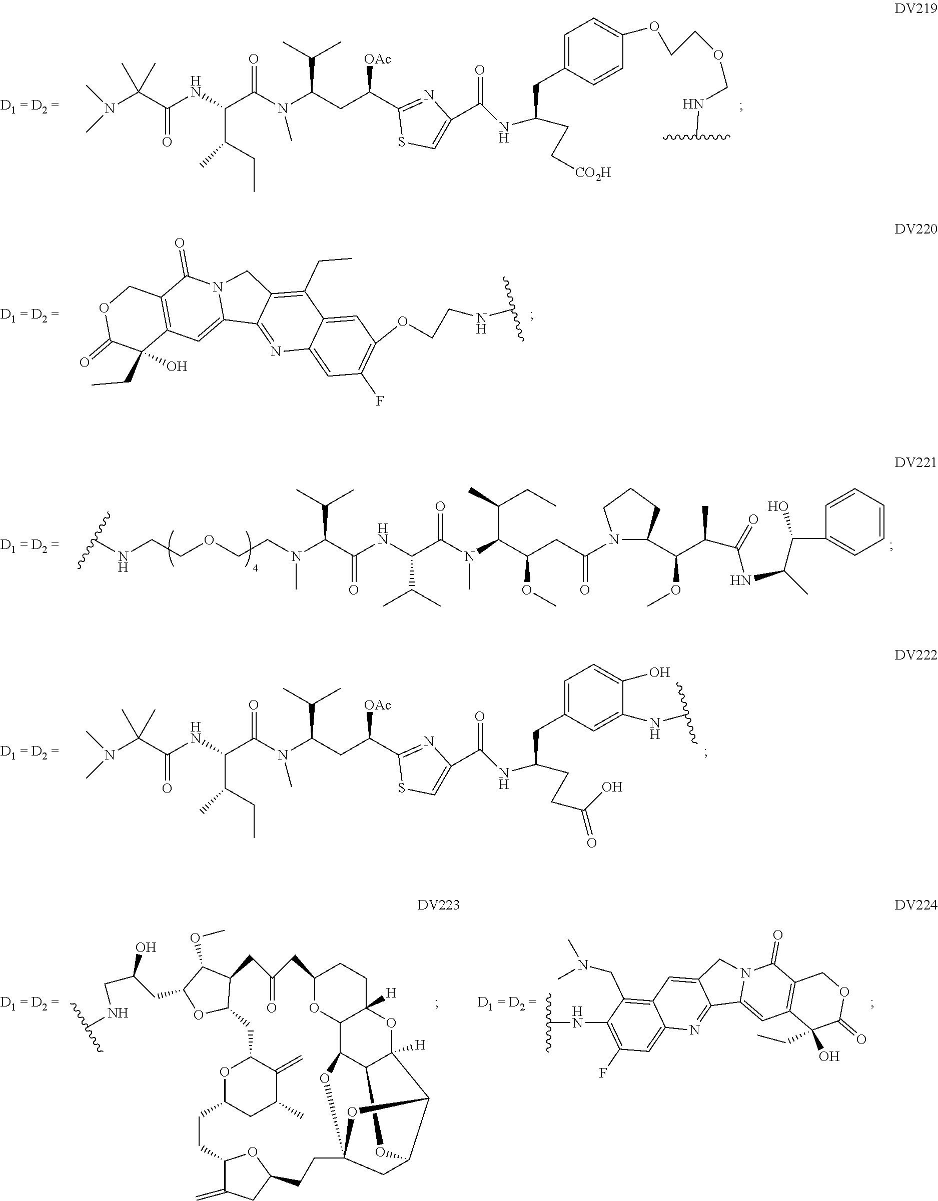

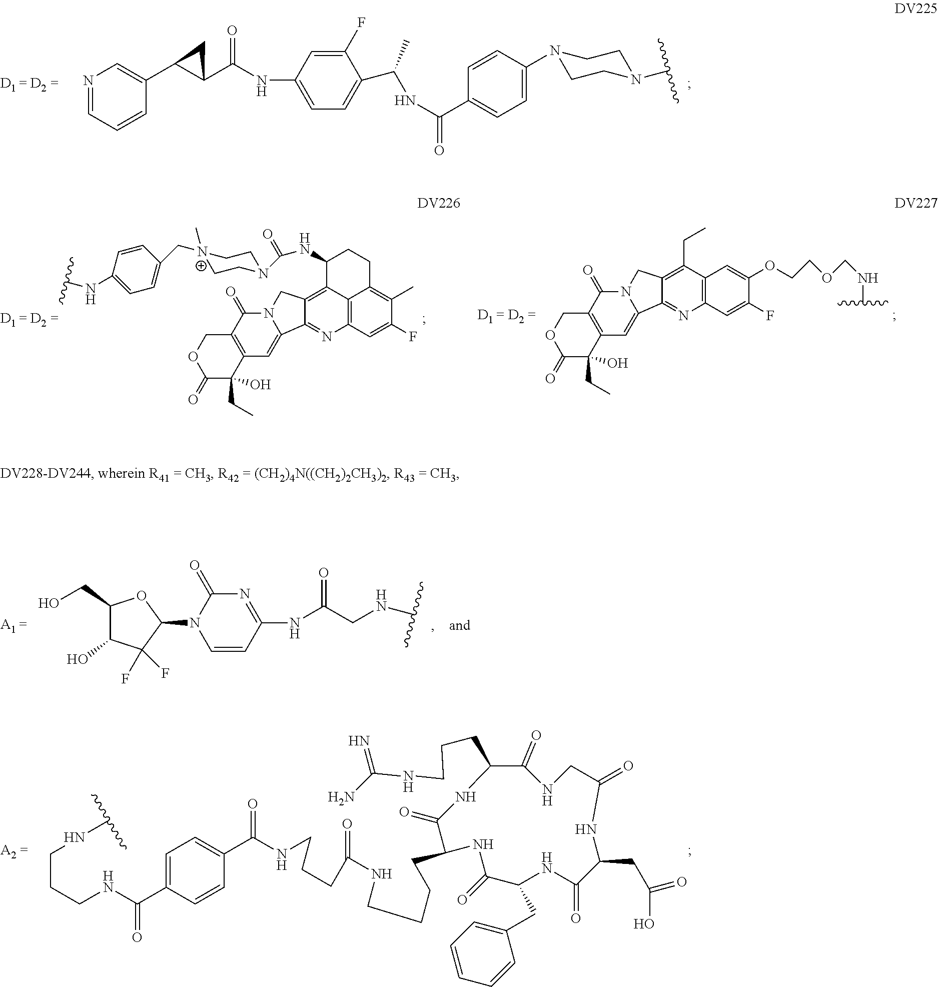

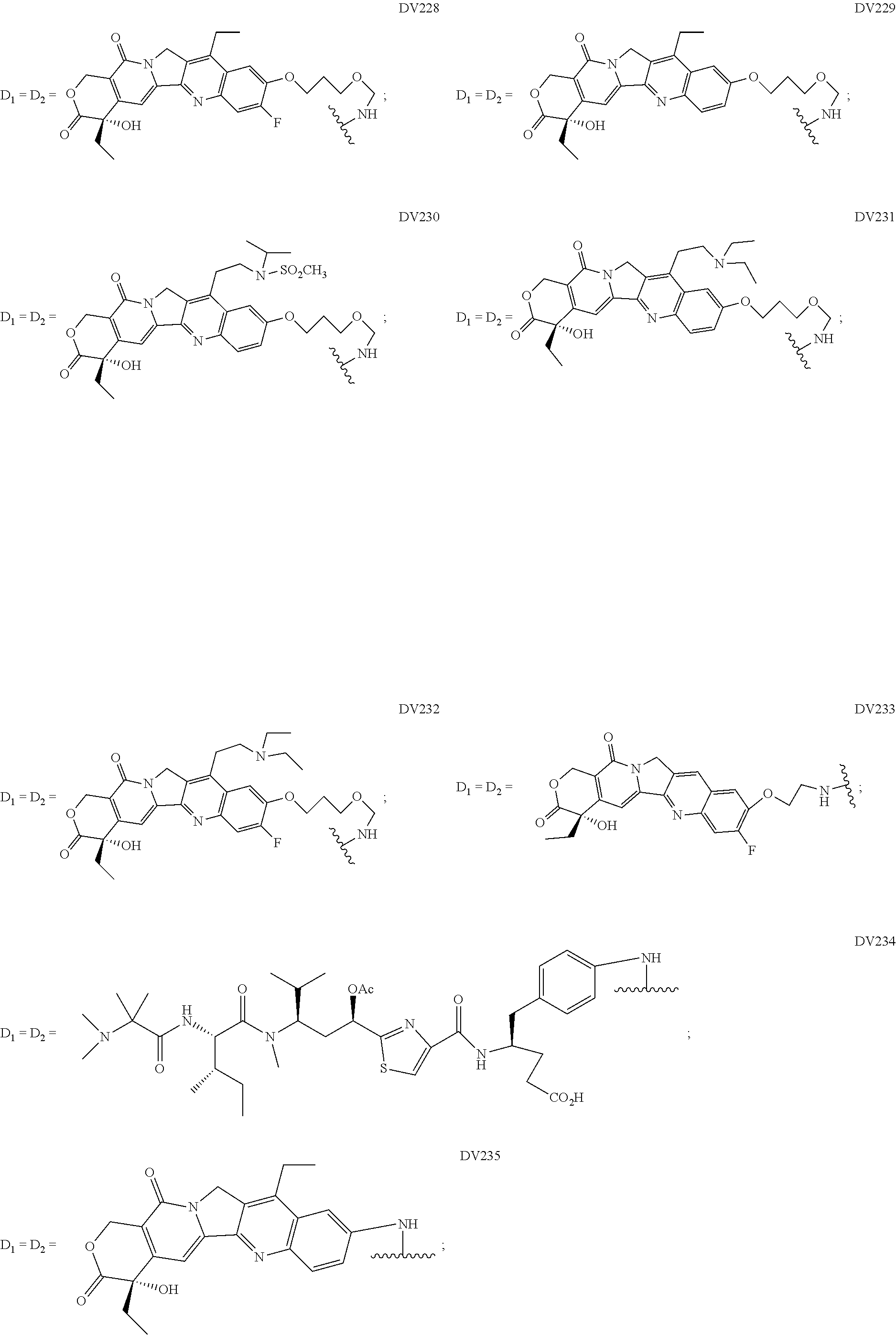

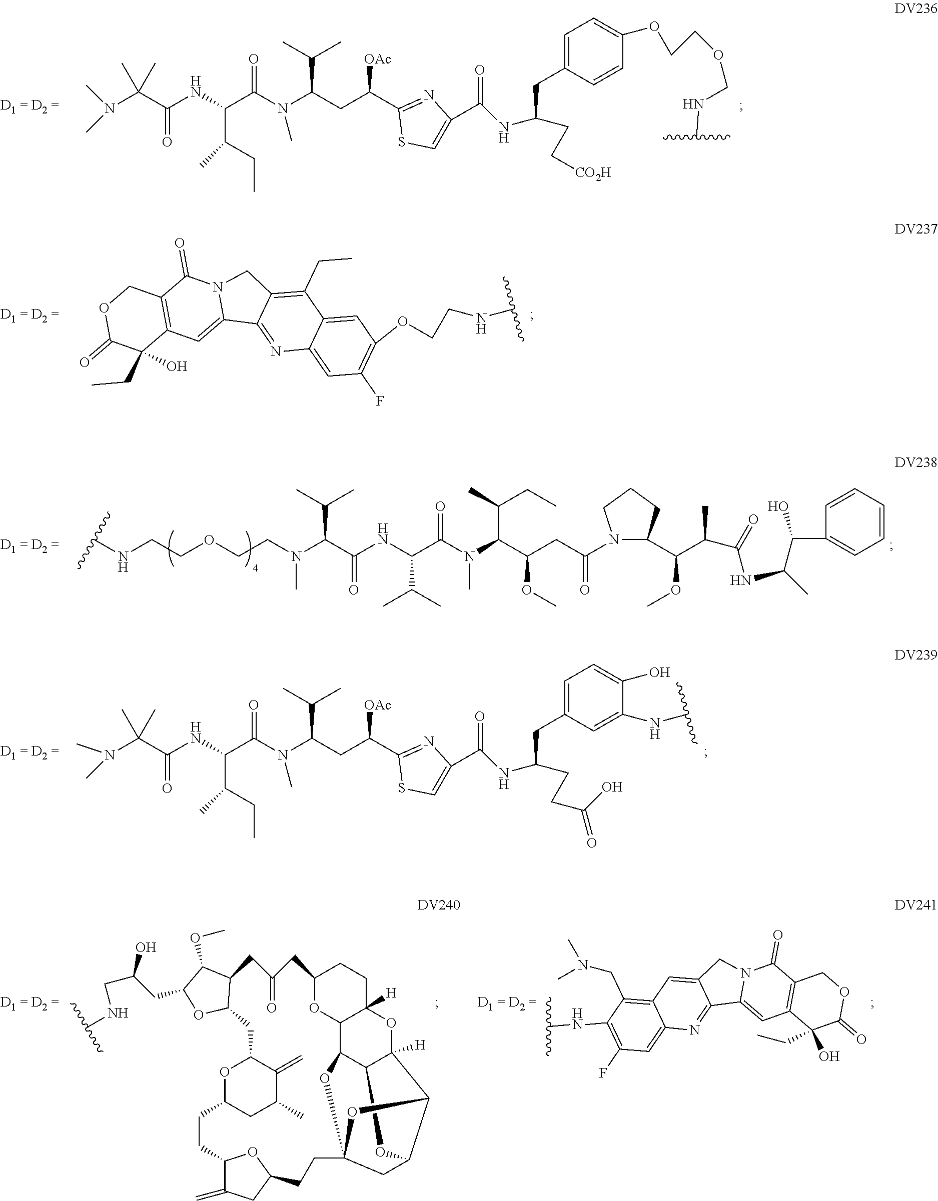

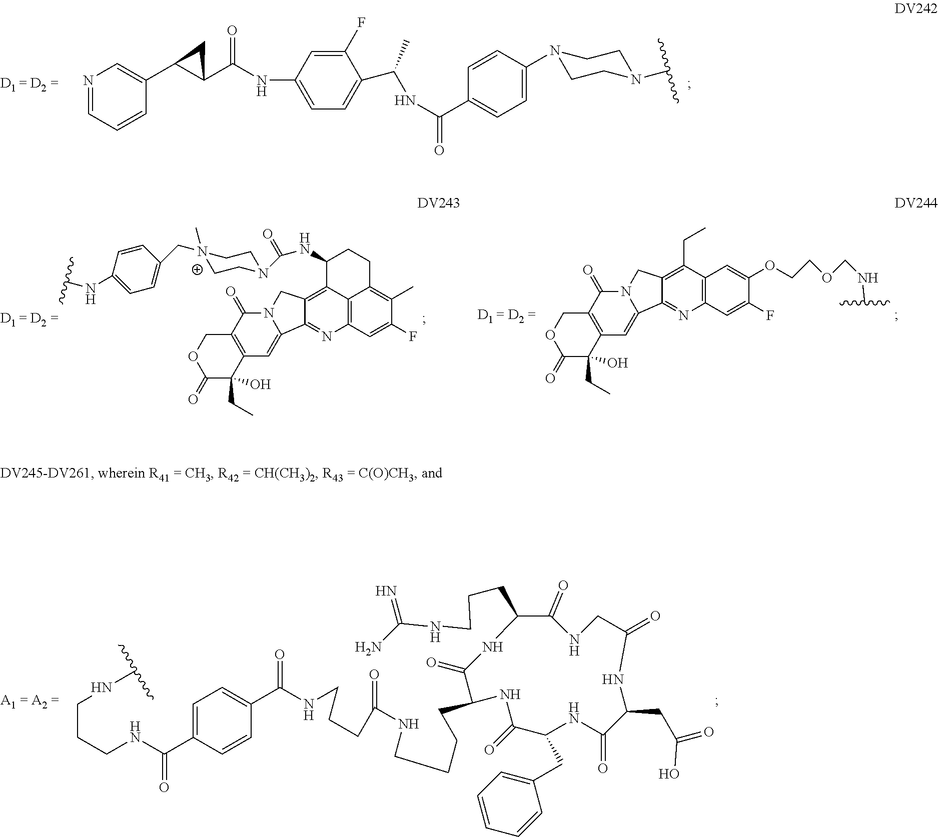

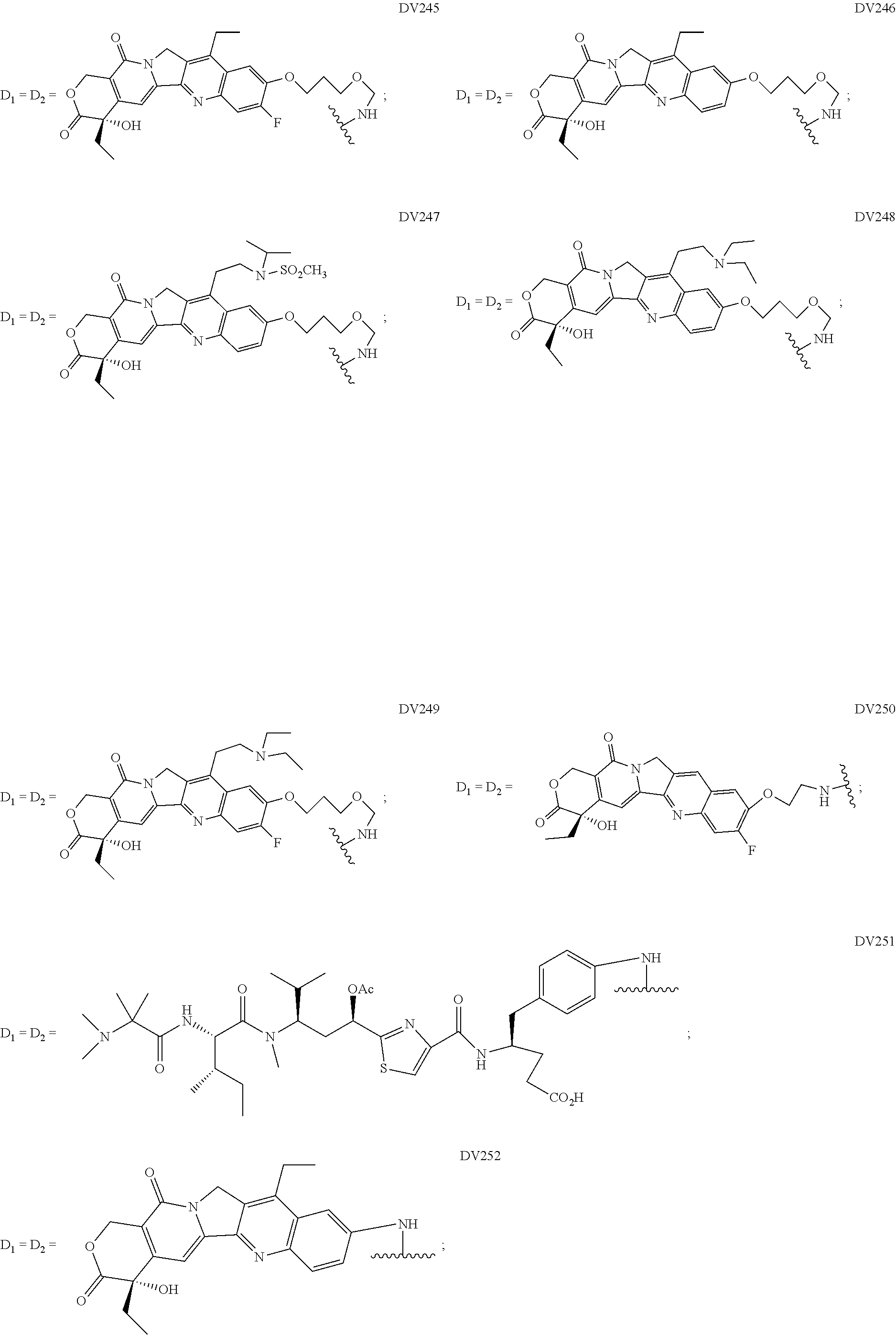

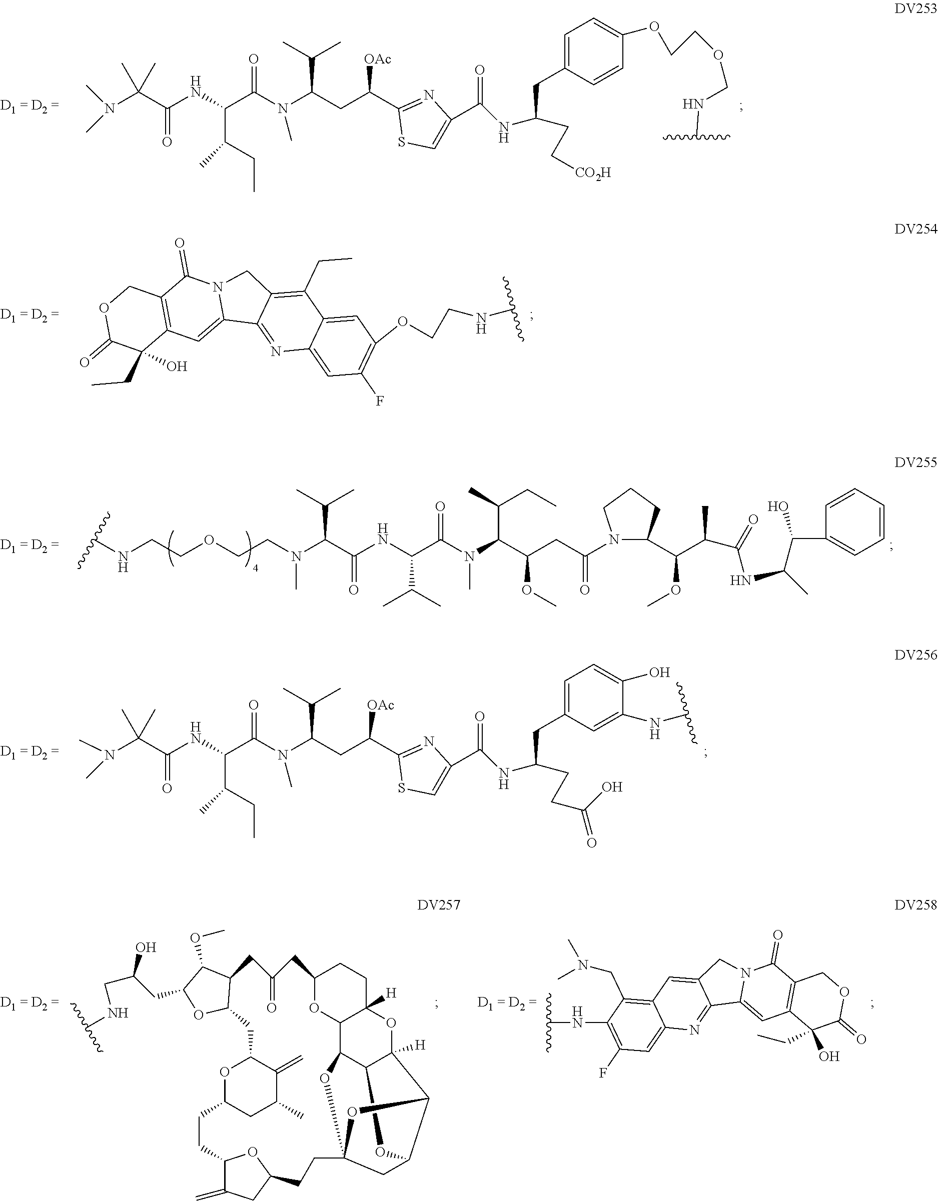

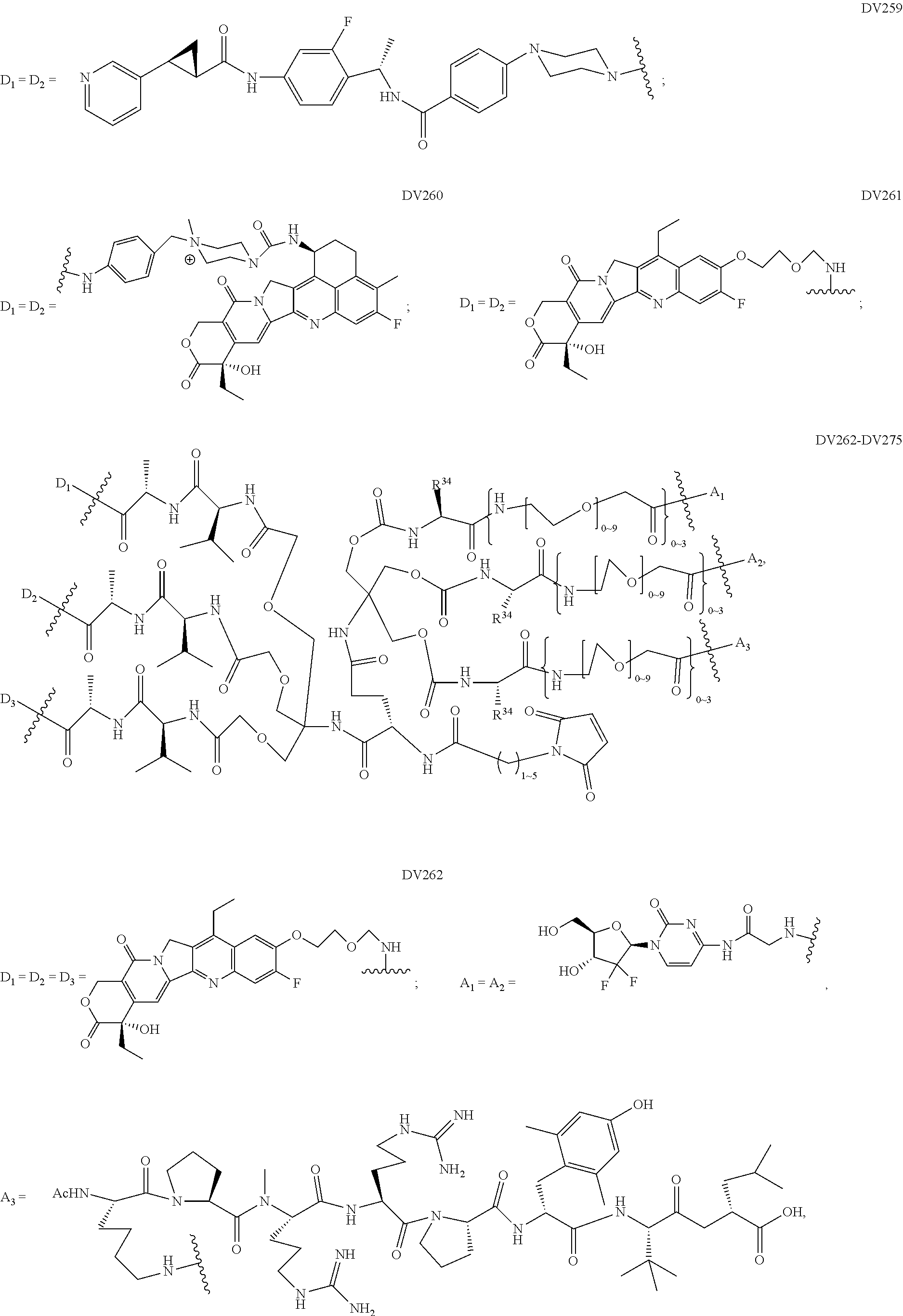

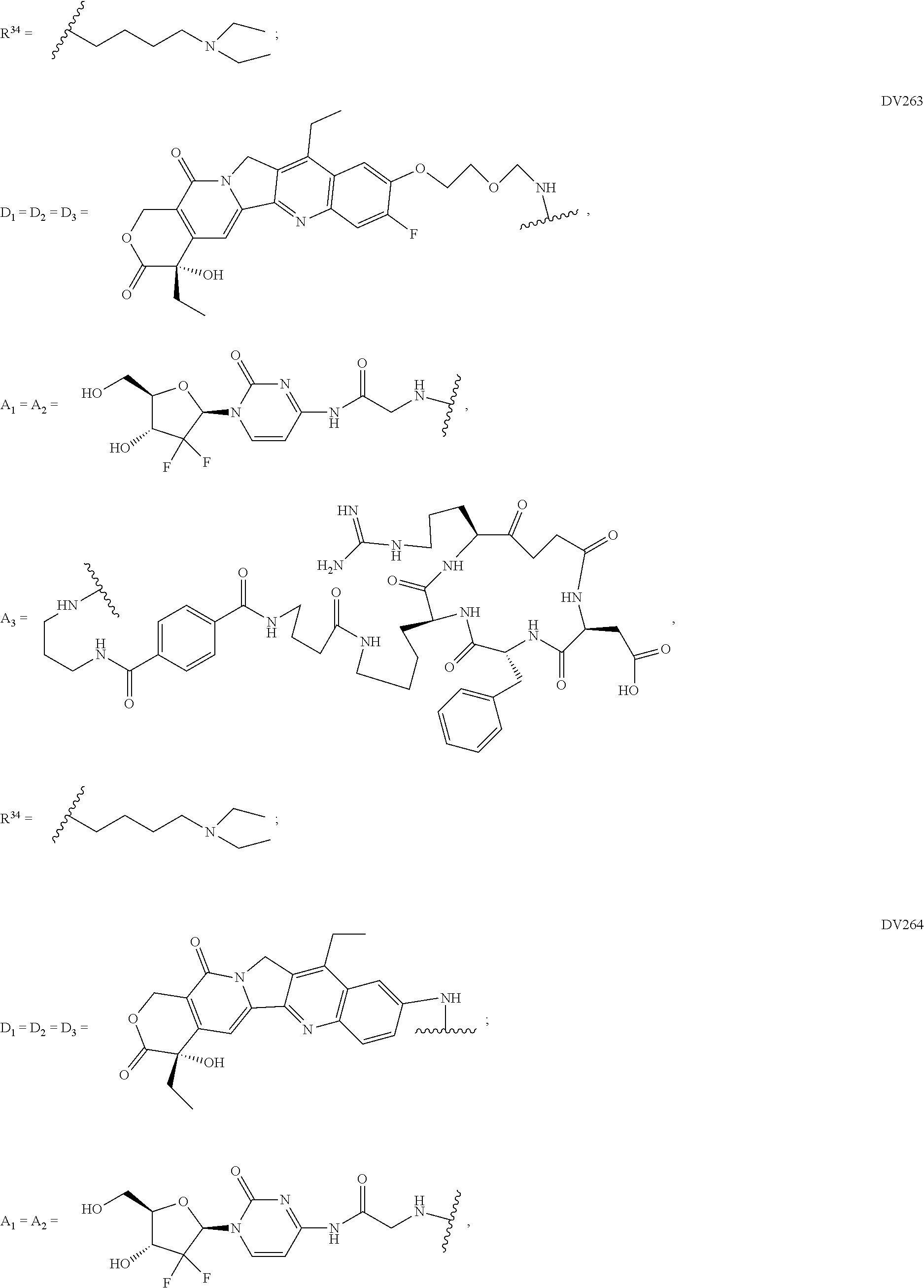

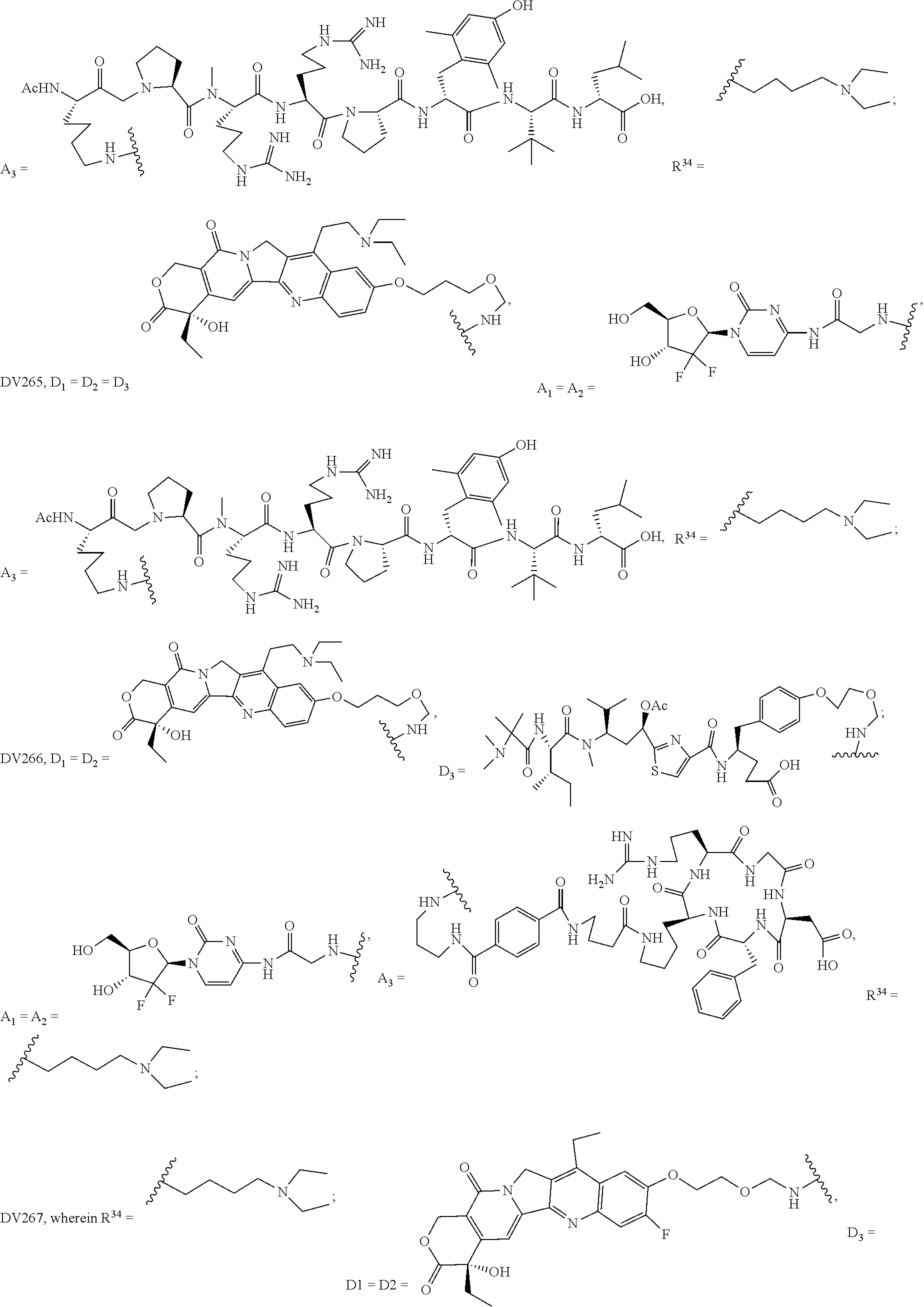

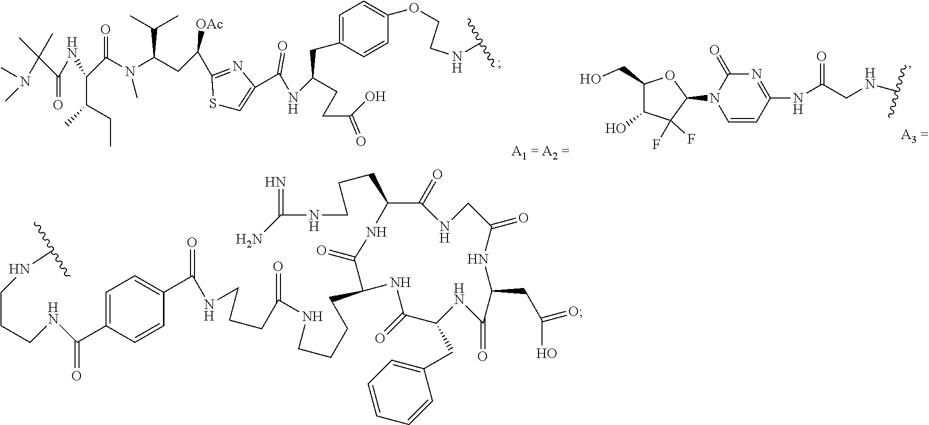

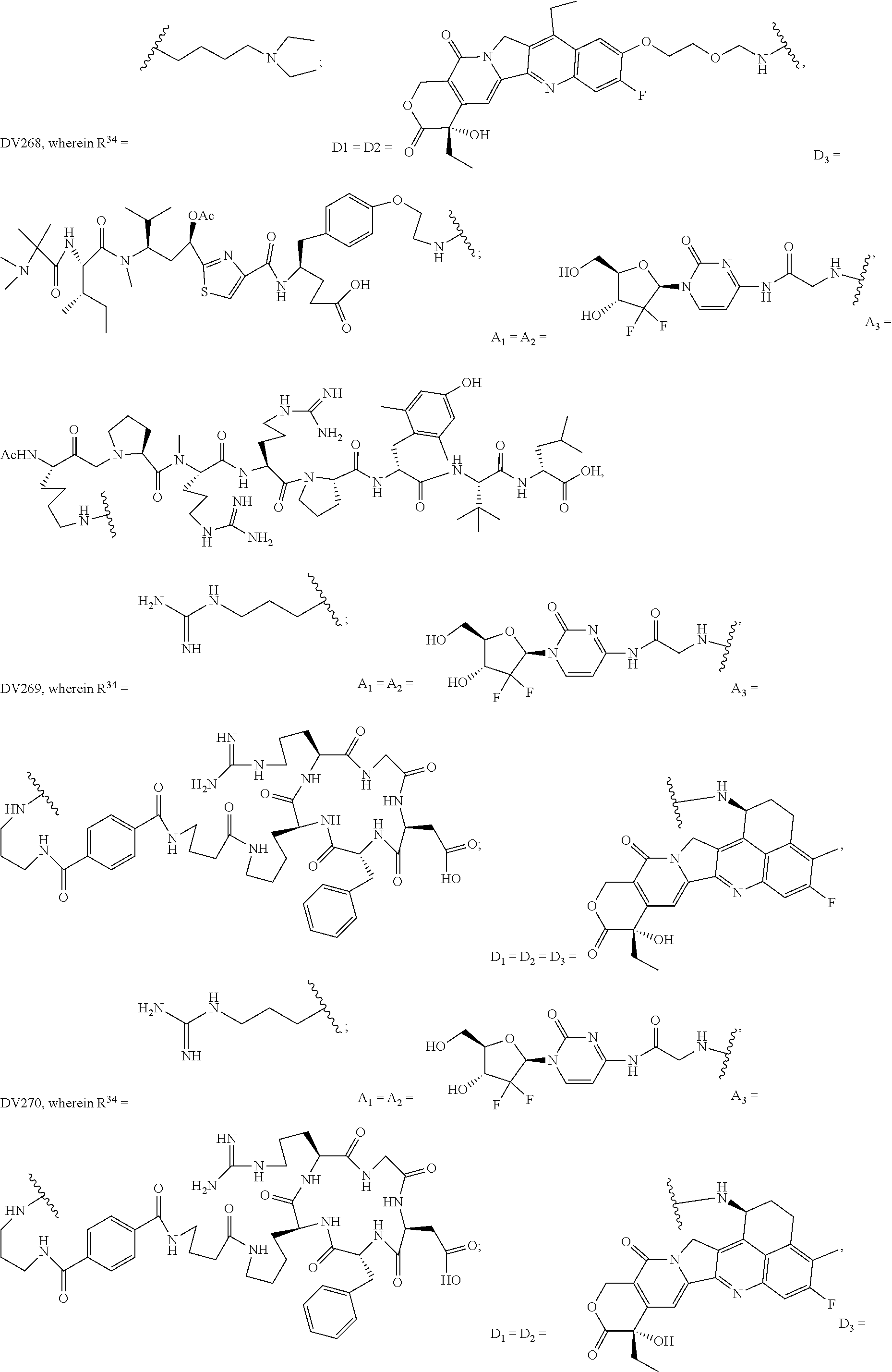

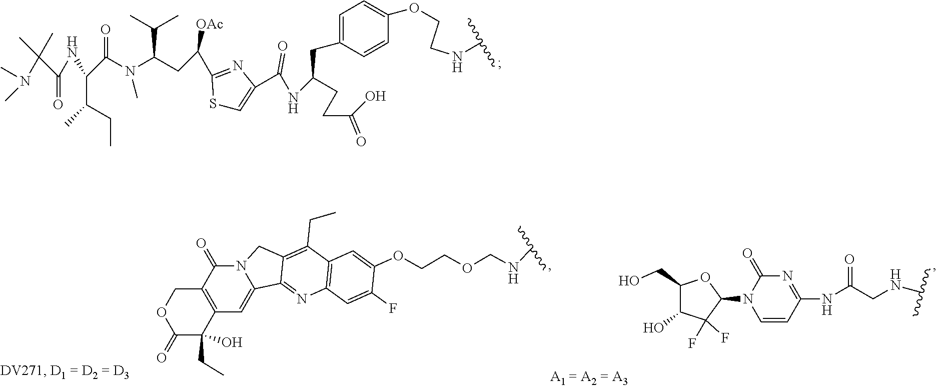

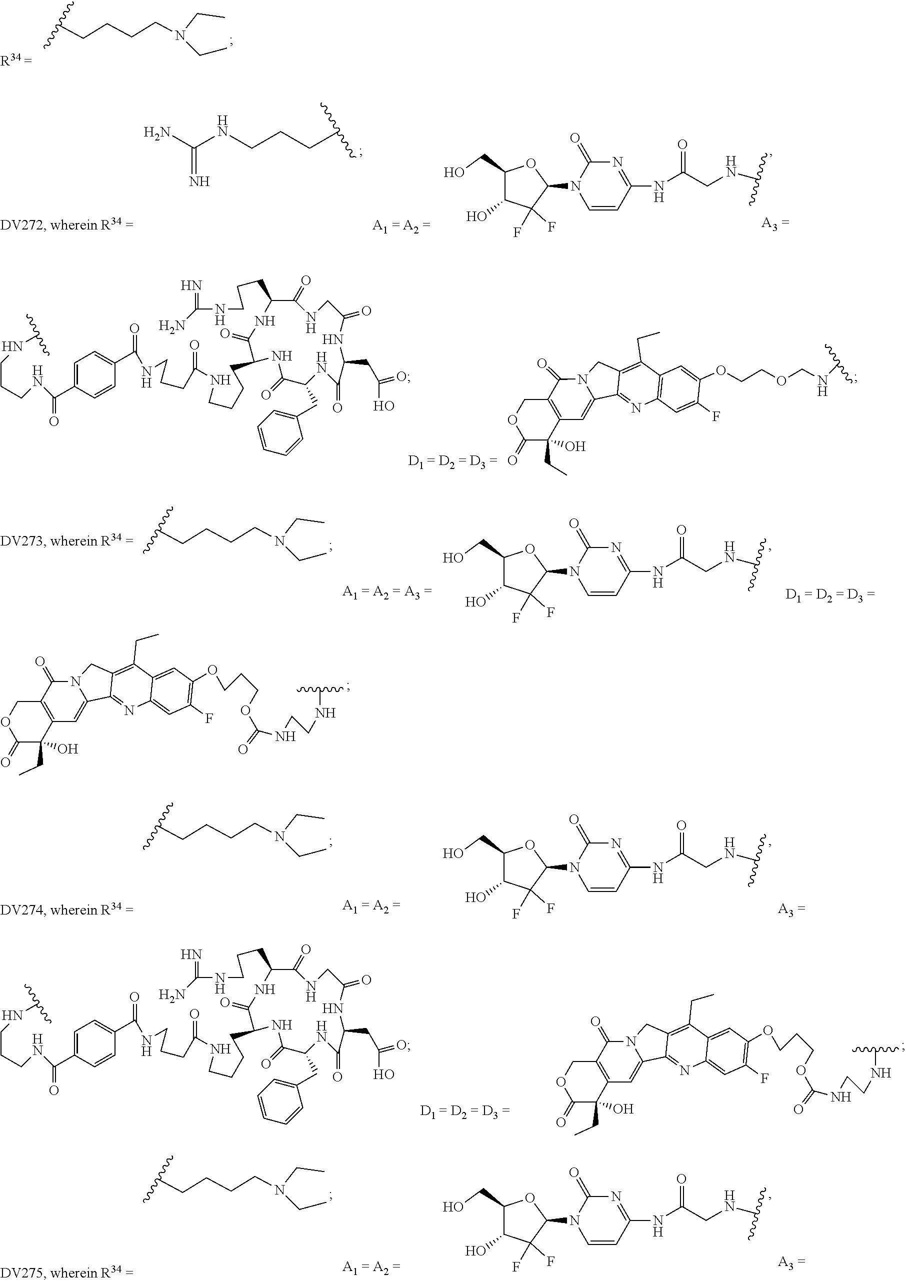

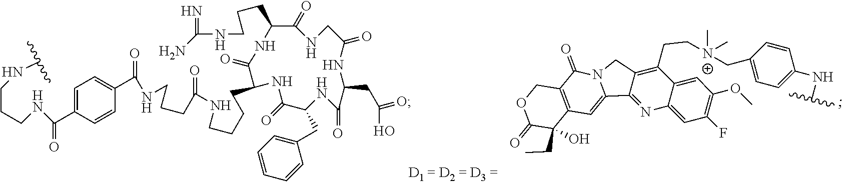

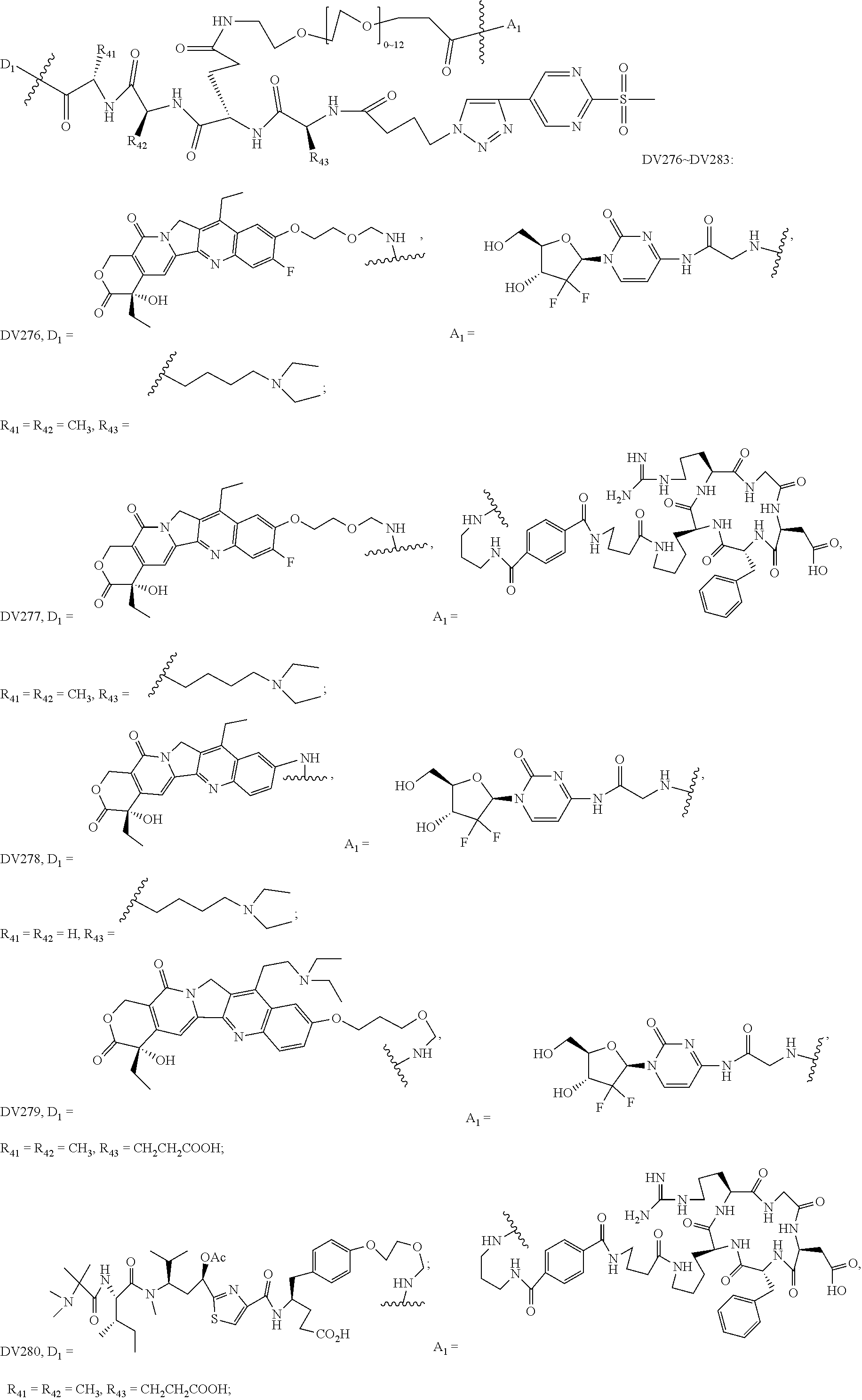

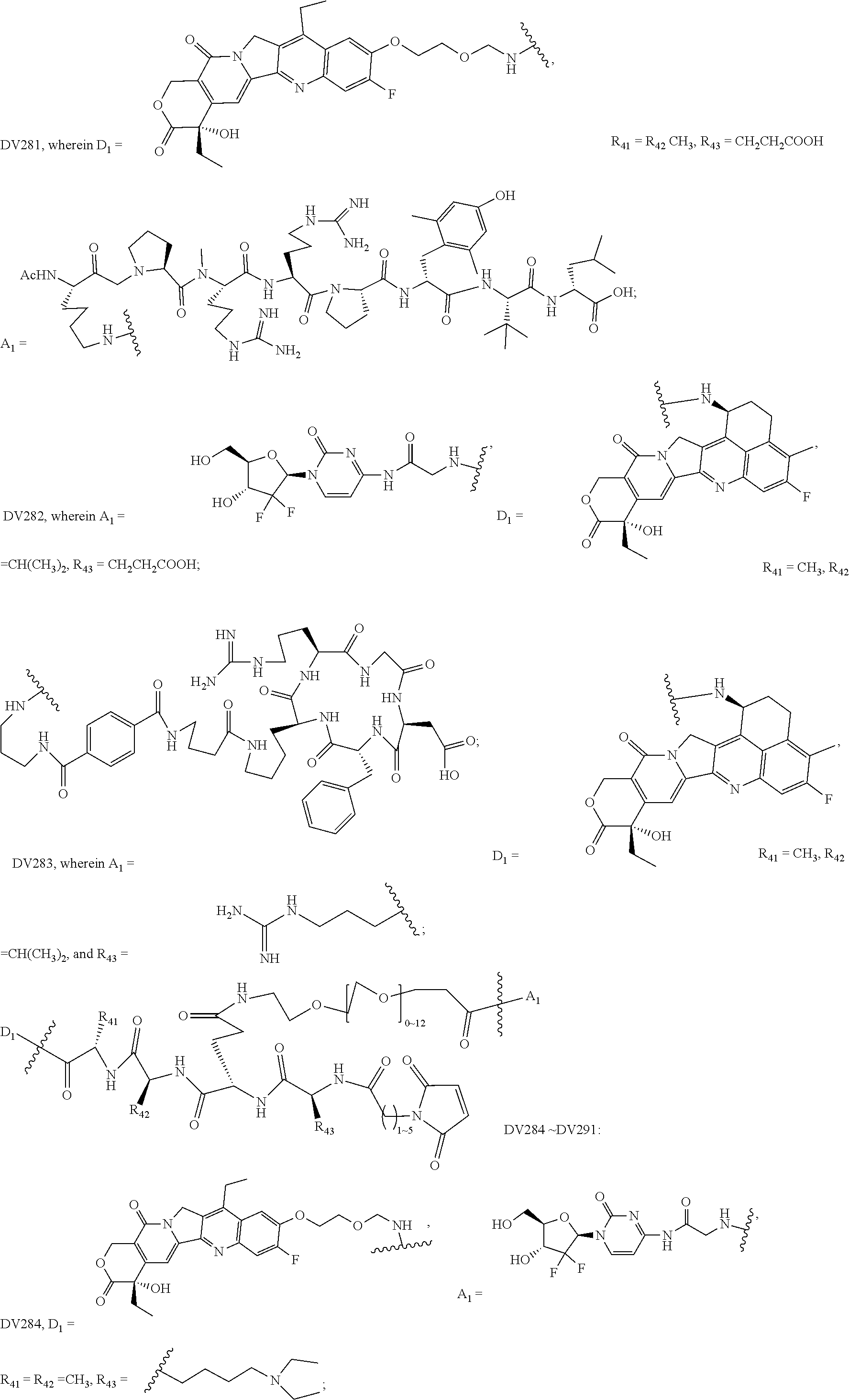

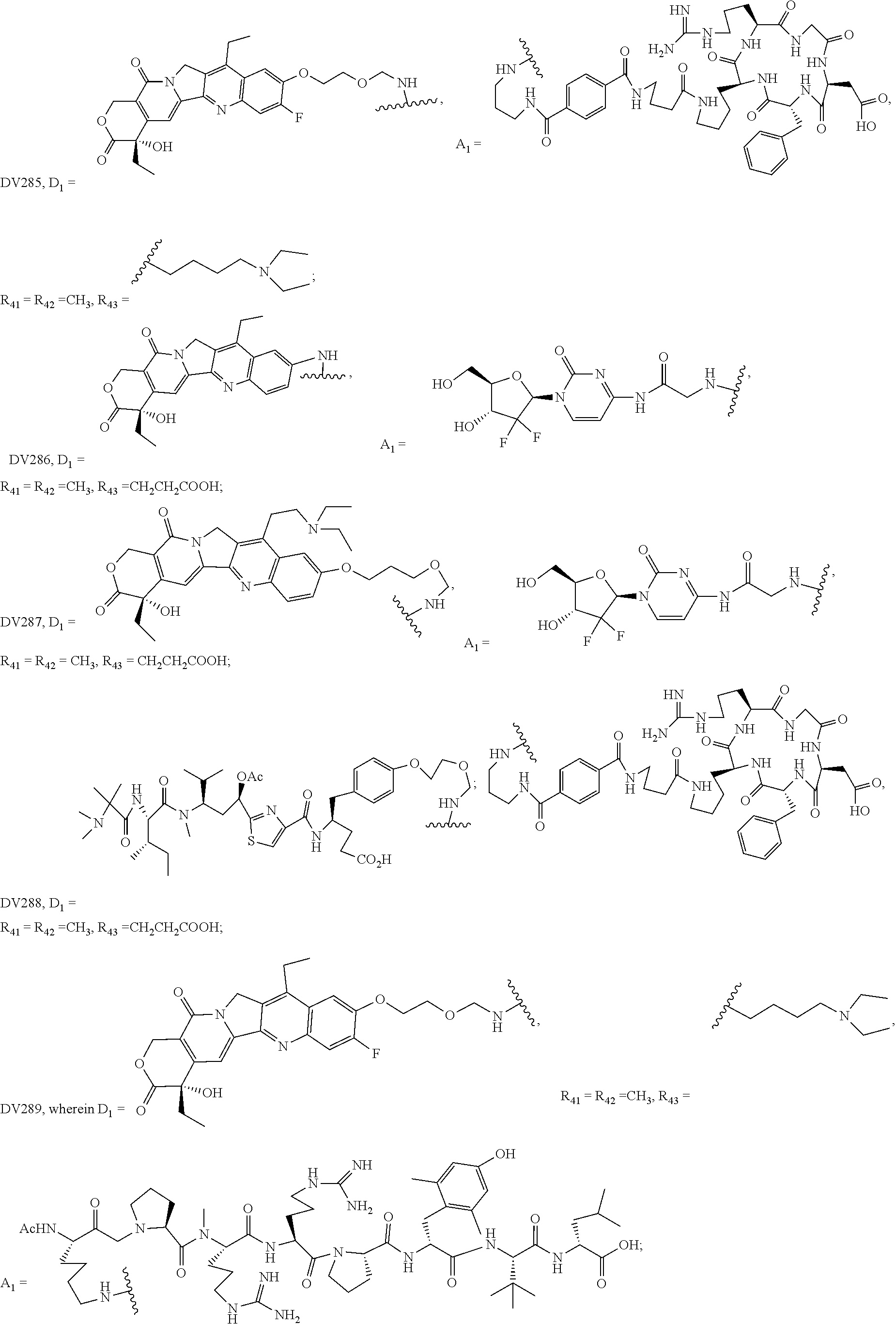

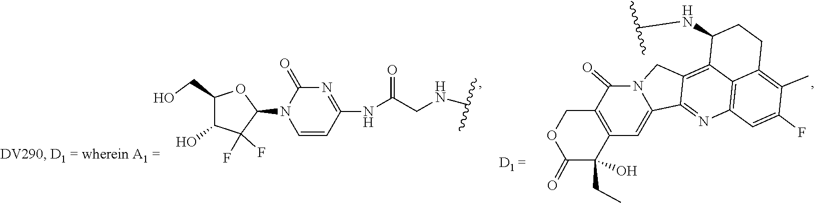

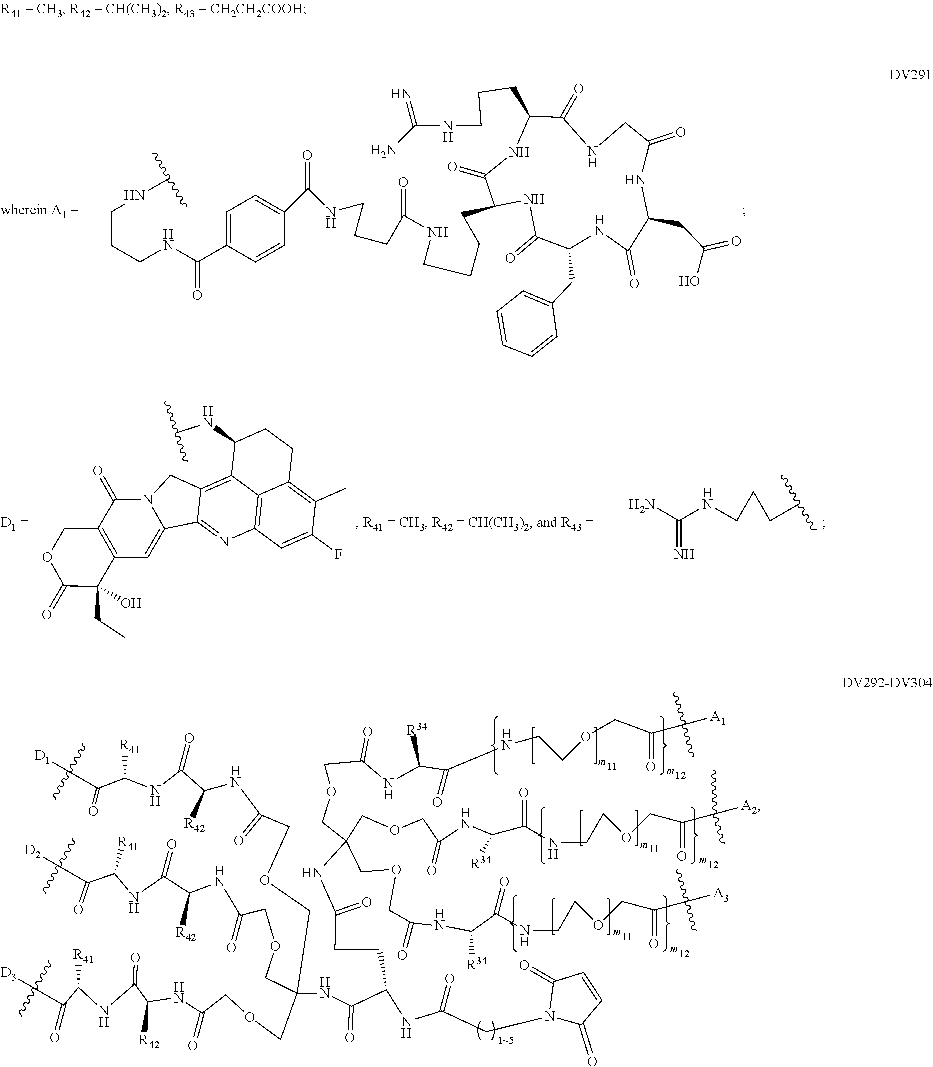

wherein R₄₁ and R₄₂ are independently H, CH₃, CH(CH₃)₂, CH(CH₃)(C₂Hs), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(—NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H₄OH; R³⁴ is H, CH₃, CH₂CH₃, C(O)H, and C(O)CH₃; D1, D2, D3 and D4 are cytotoxic drug described in this application, and A₁, A₂ and A₃ are small molecular ligands or drugs described in this application; mn is a number of 0, 1, 2, 3, 4, 5, 6, 7, 8, 9 and m₁₂ is a number of 0, 1, 2, 3 and 4; when number is 0 for the brackets, it means the structure in the brackets can be omitted (crossed out),

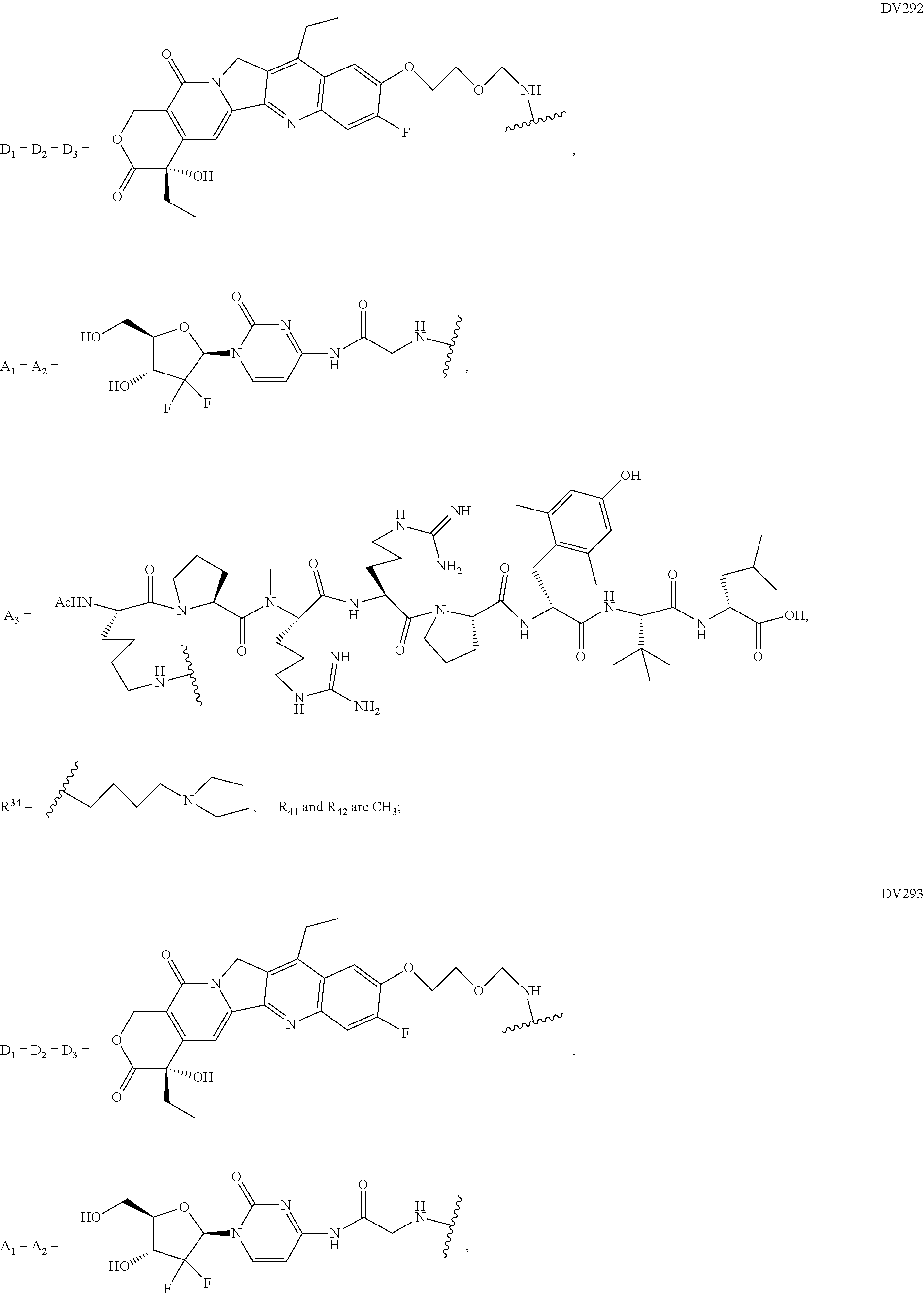

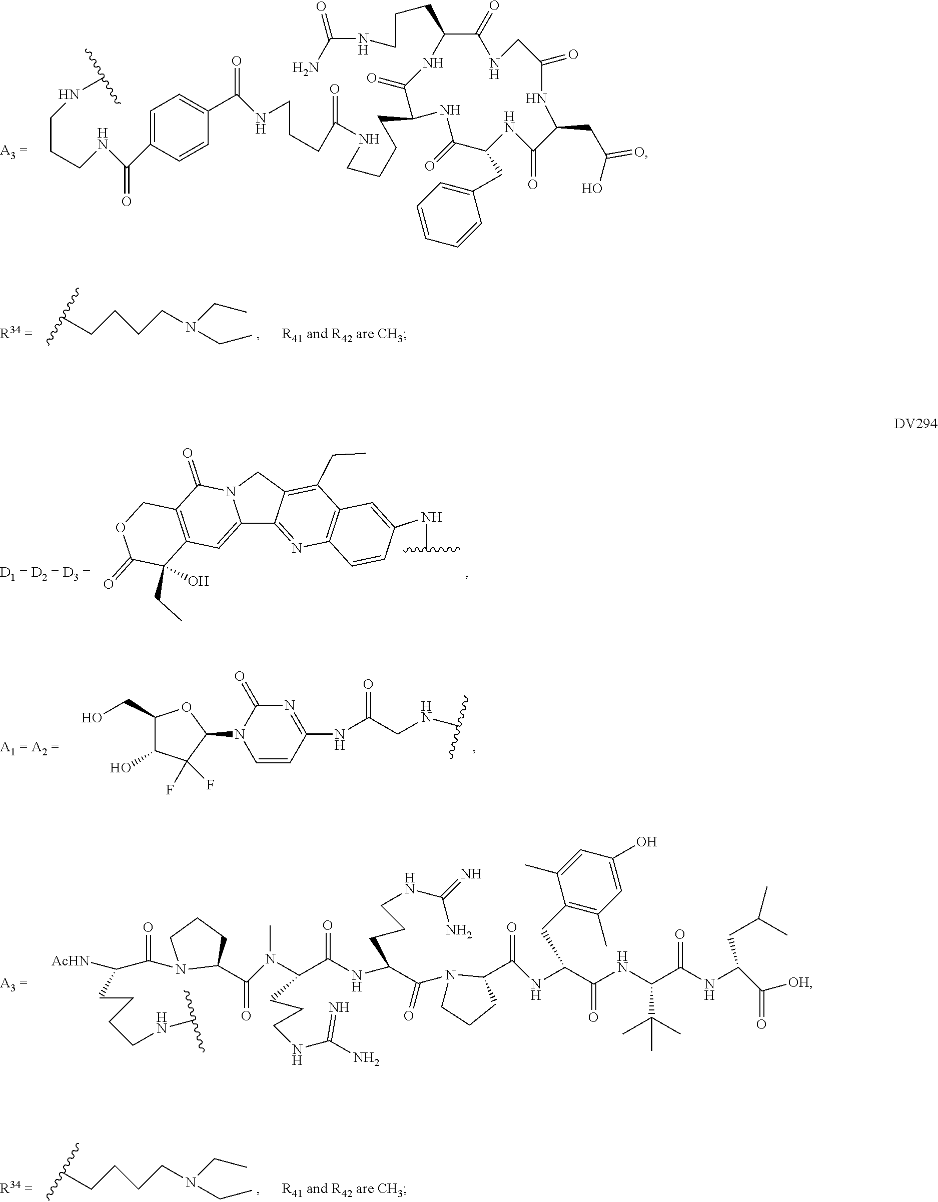

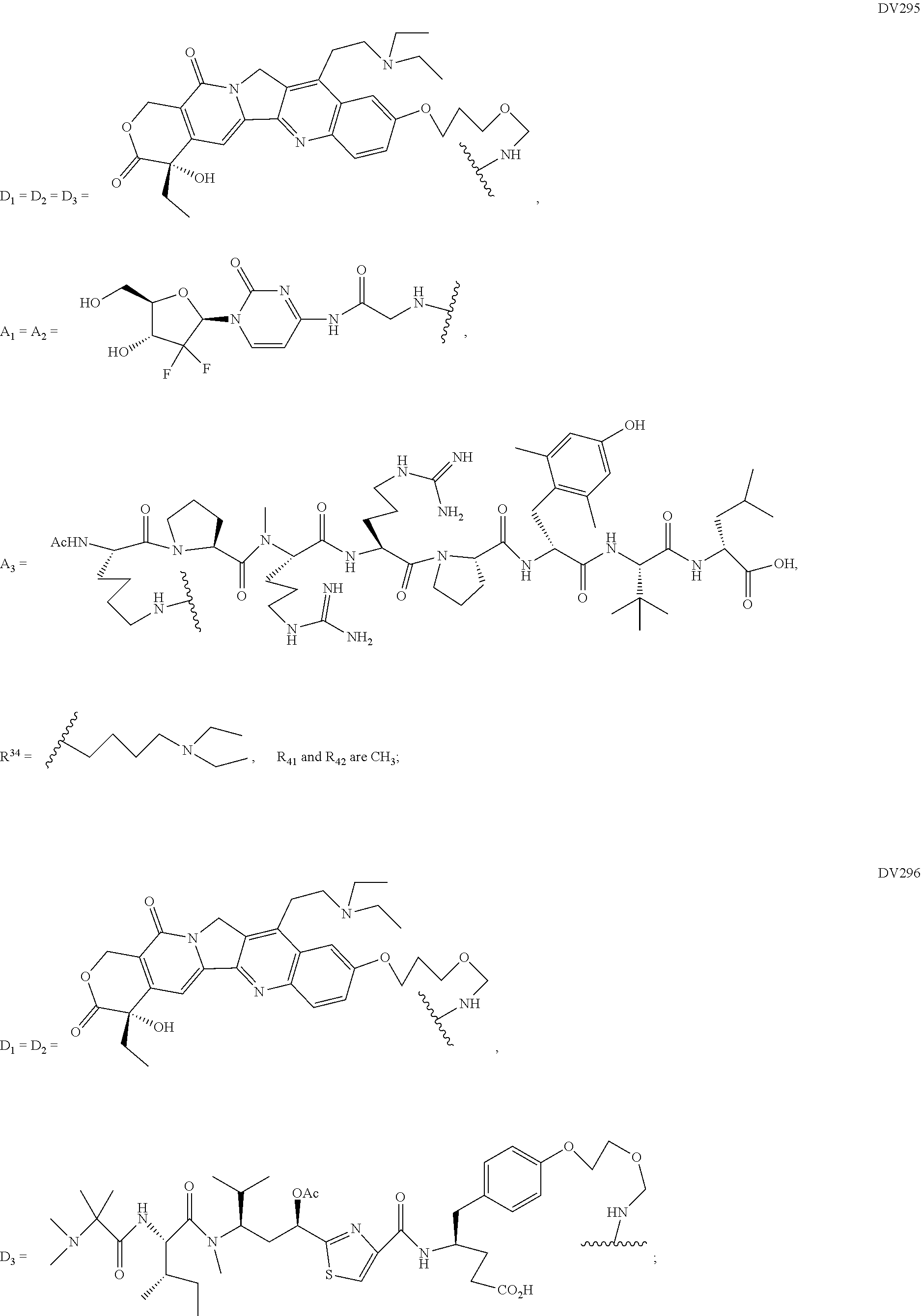

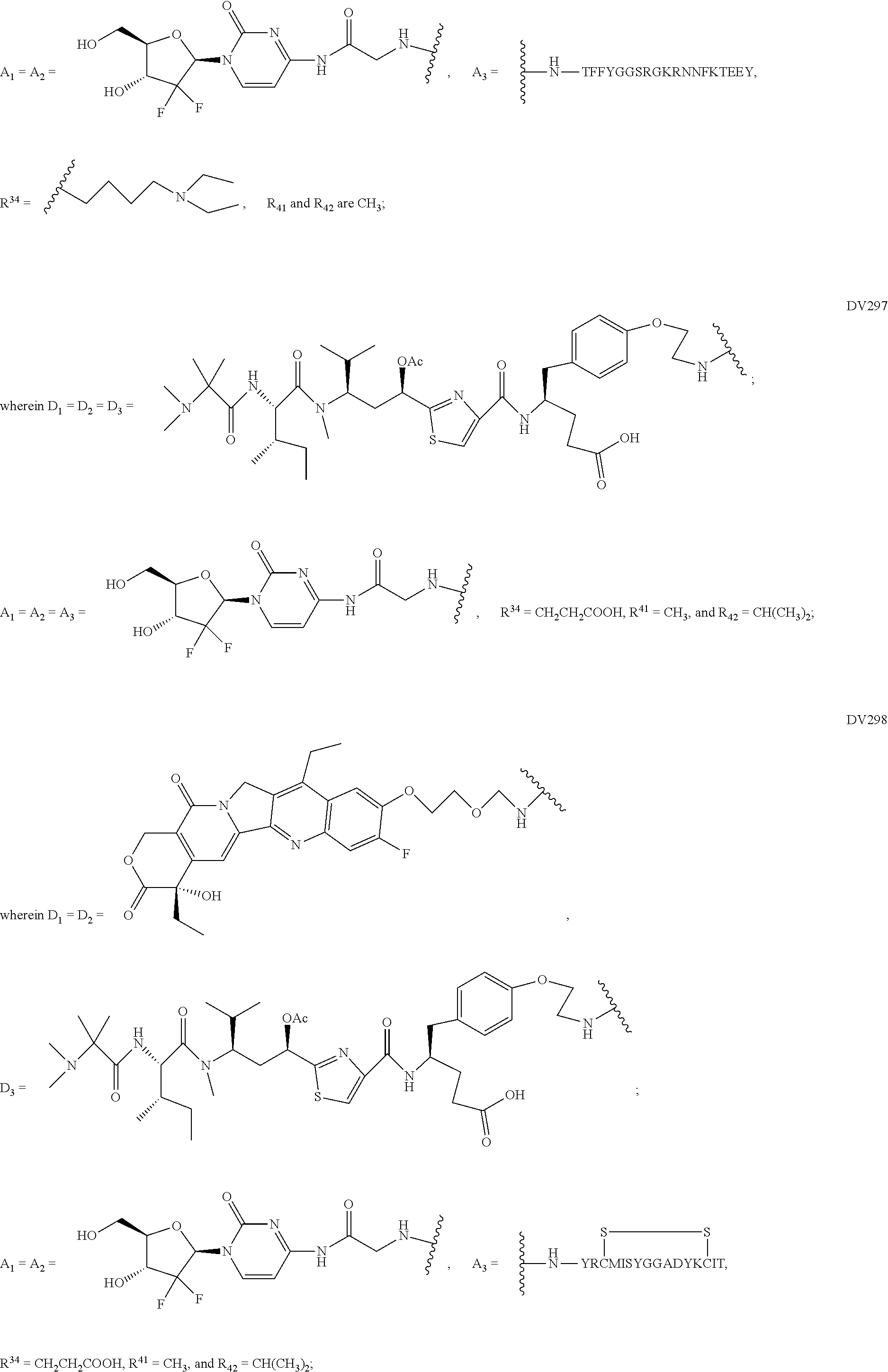

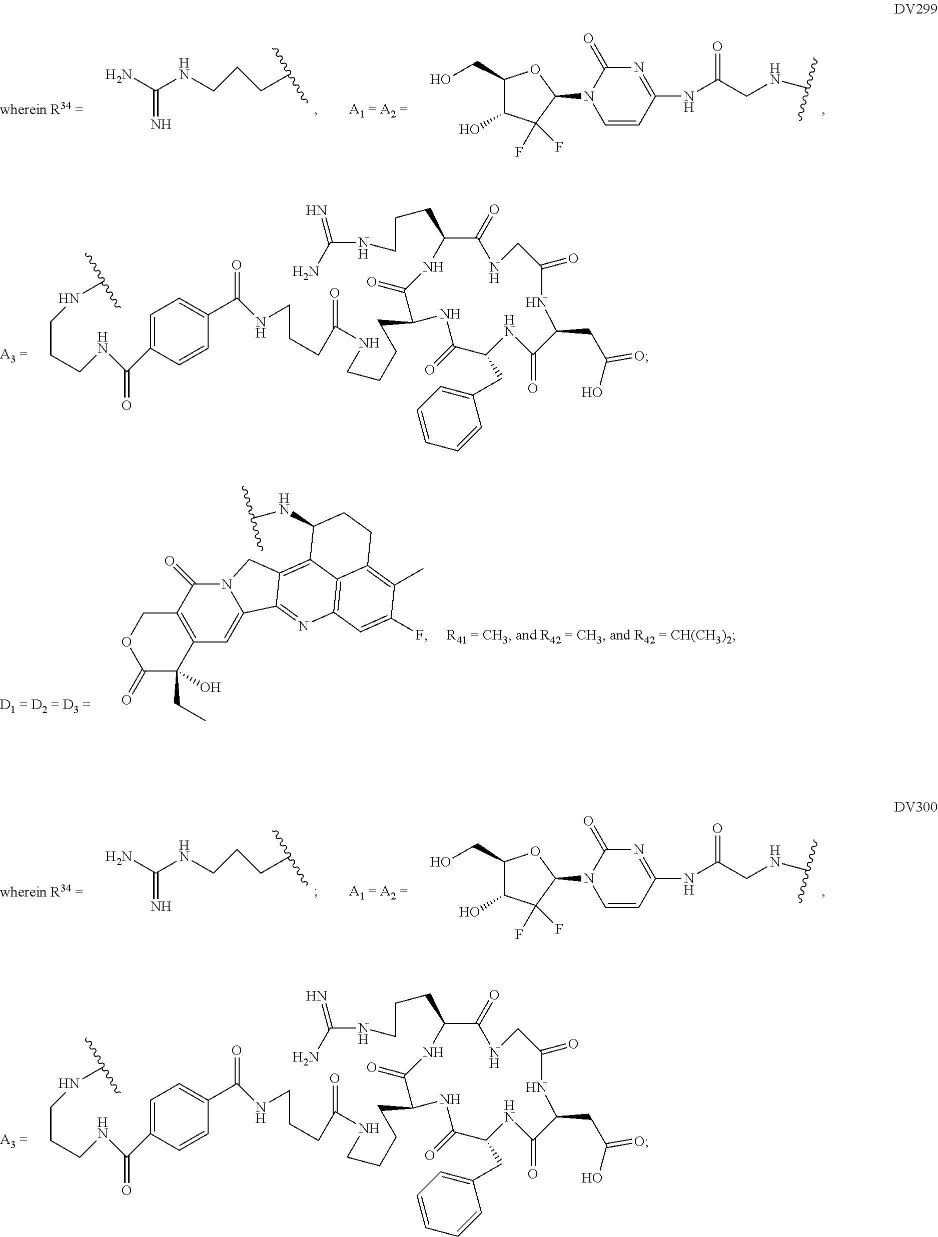

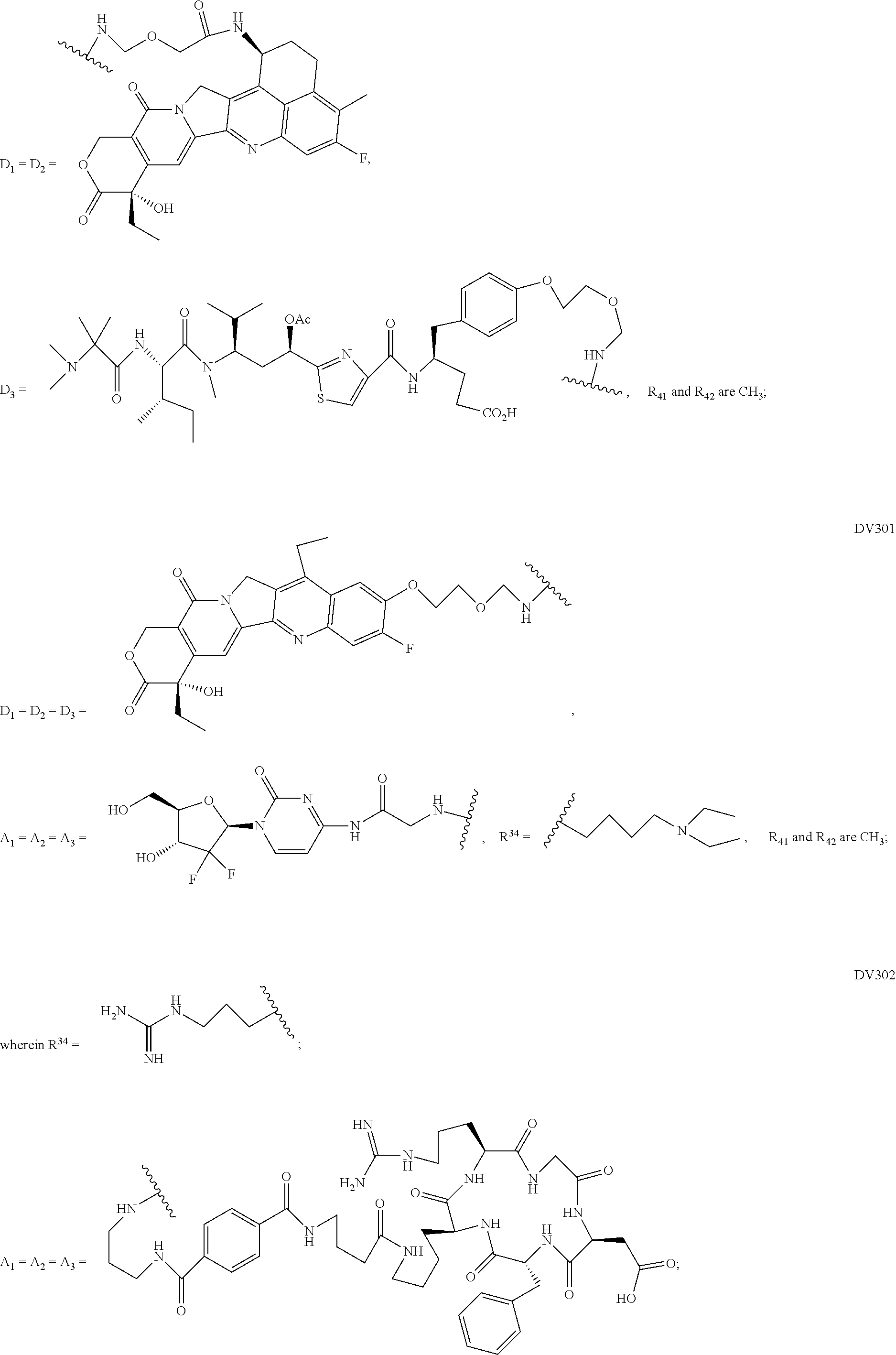

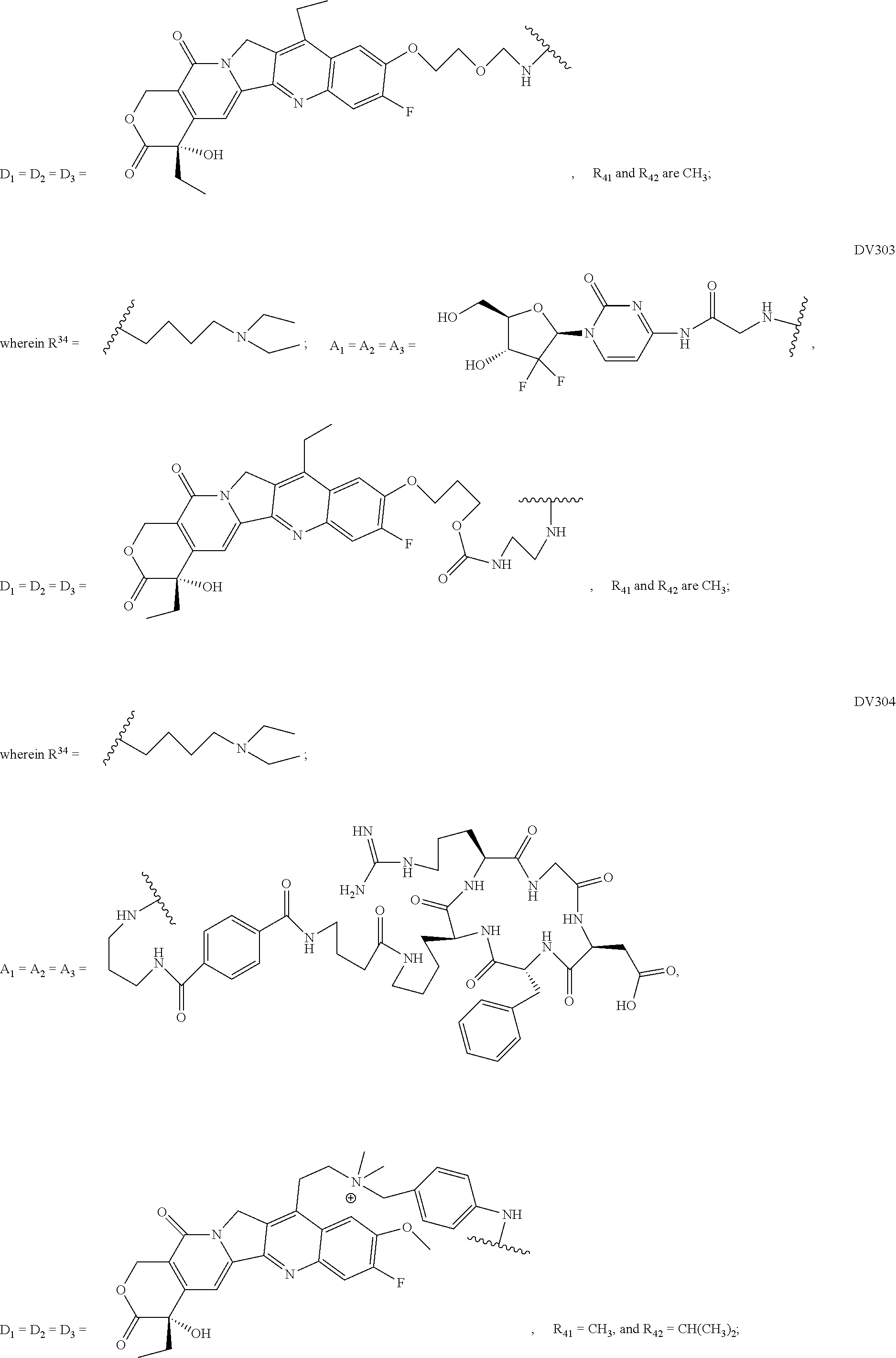

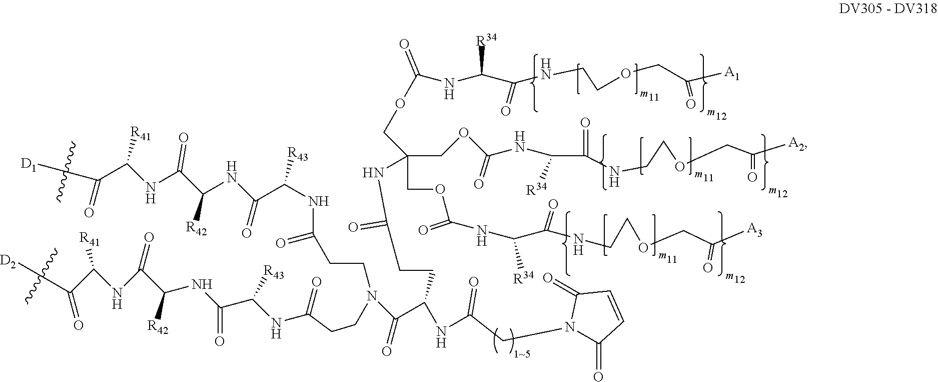

wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(—NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H₄OH; R³⁴ is H, CH₃, CH₂CH₃, C(O)H, and C(O)CH₃; D1, D2, D3 and D4 are cytotoxic drug described in this application, and A₁, A₂ and A₃ are small molecular ligands or drugs described in this application; mi is a number of 0, 1, 2, 3, 4, 5, 6, 7, 8, 9 and m₁₂ is a number of 0, 1, 2, 3 and 4; when number is 0, it means the structure in the brackets can be omitted (crossed out).

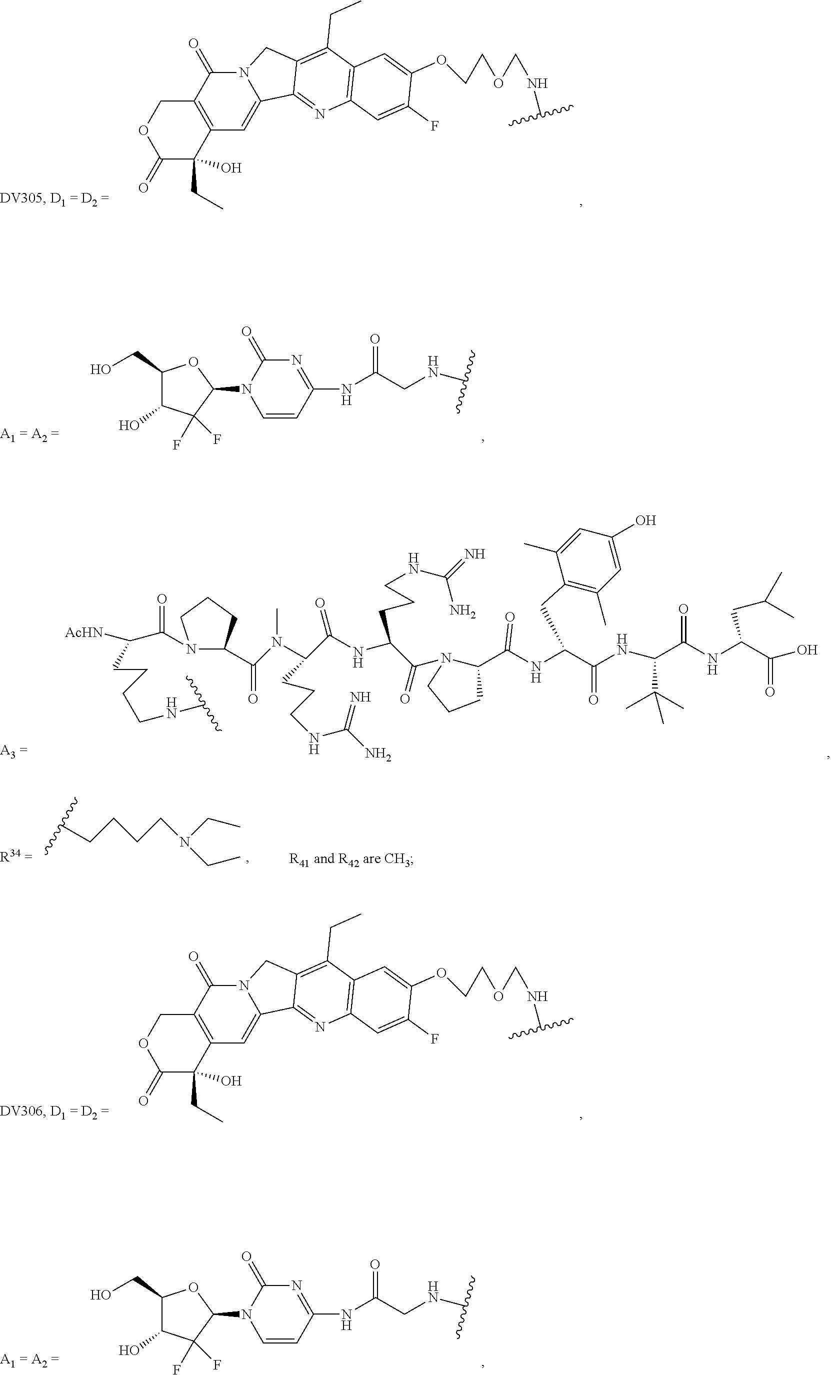

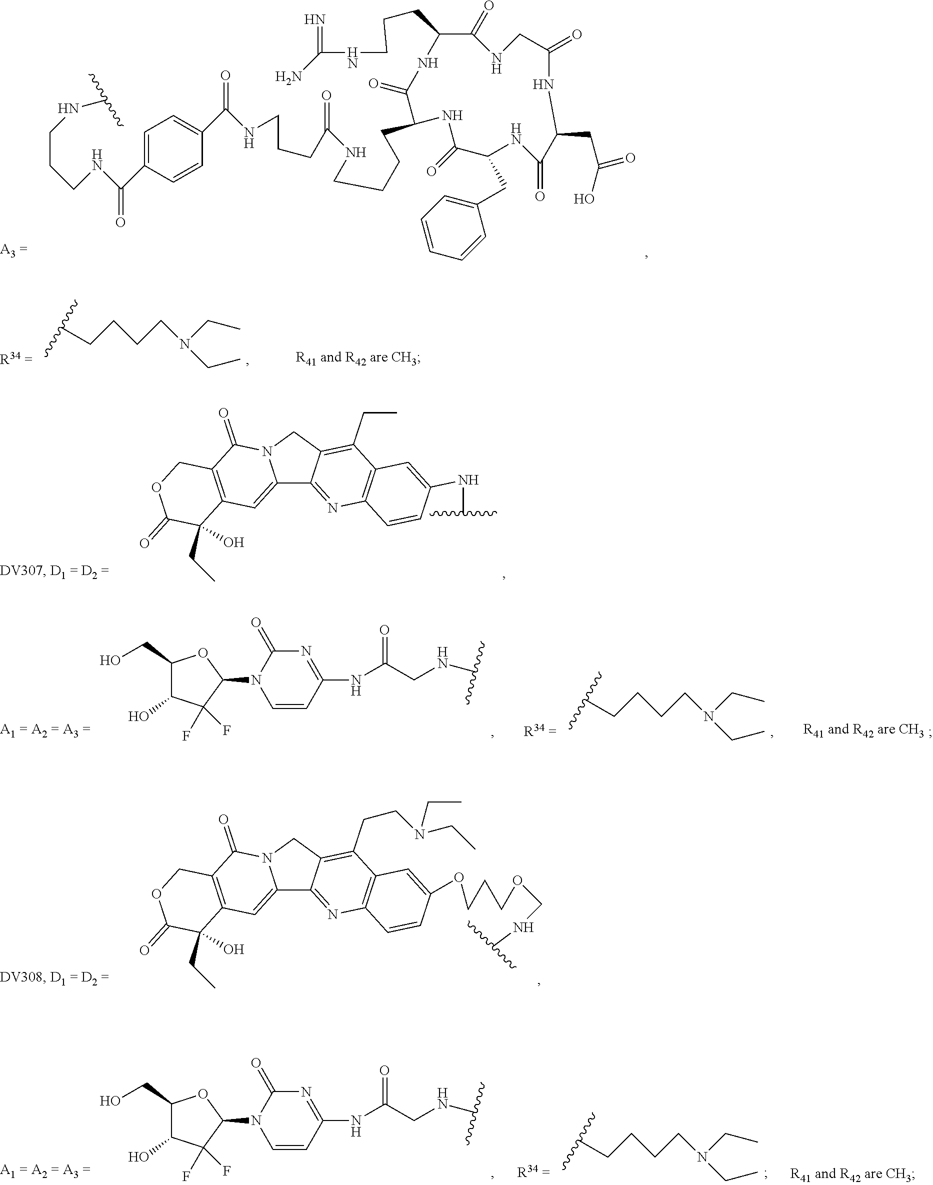

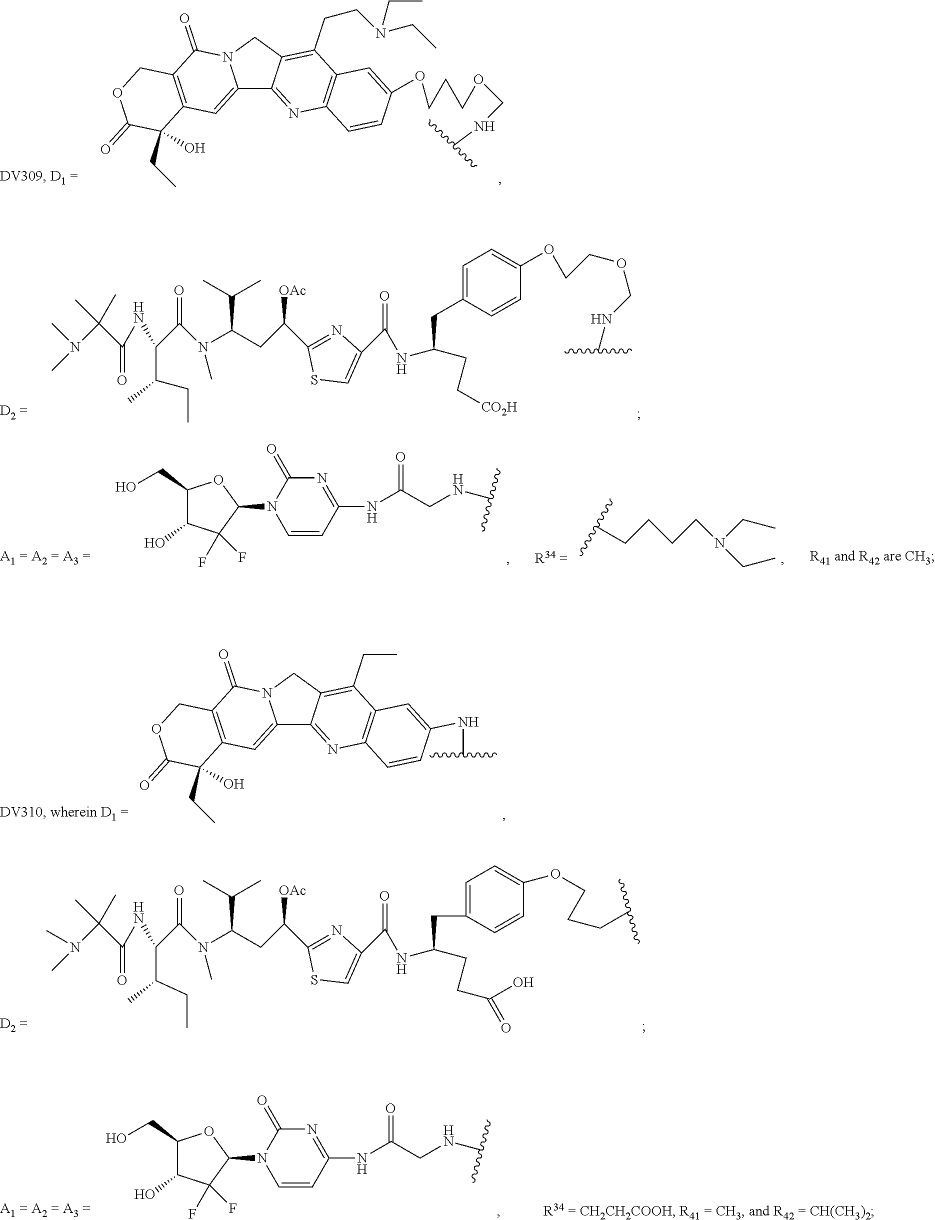

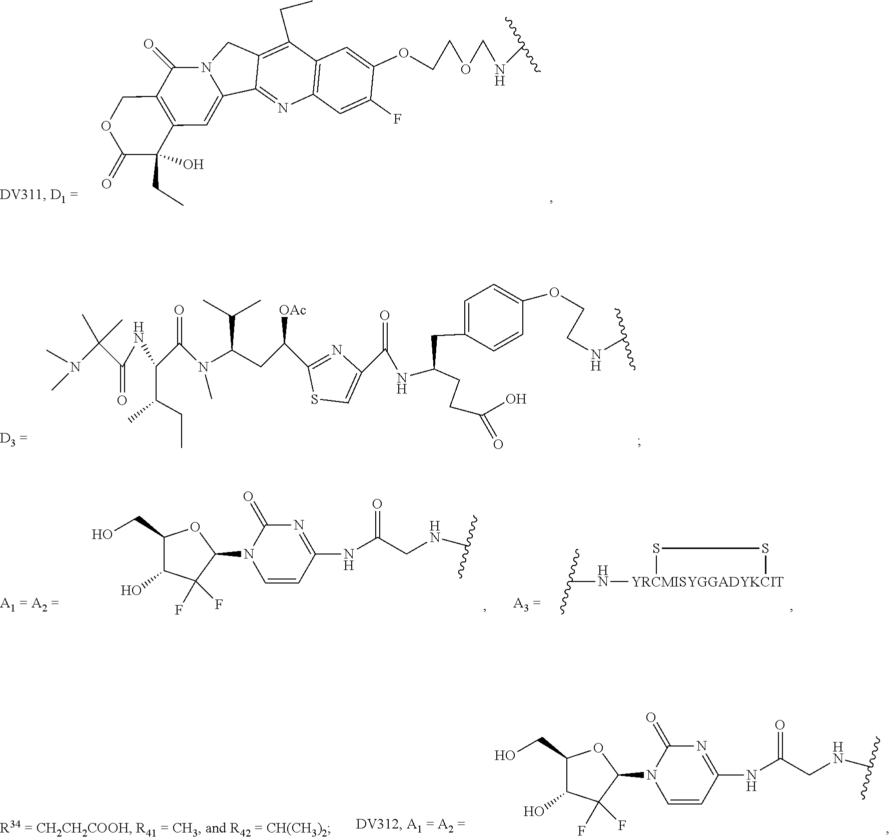

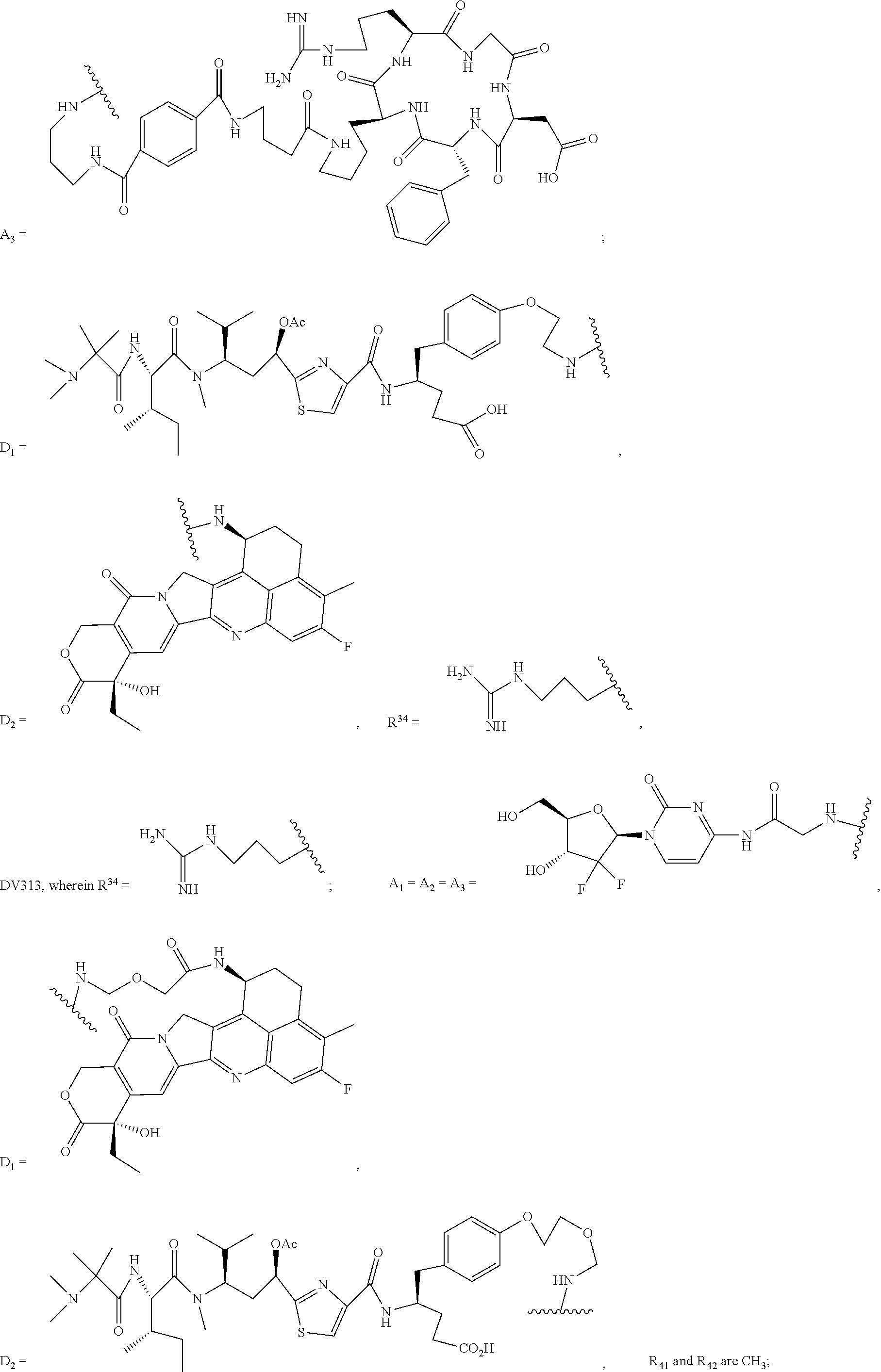

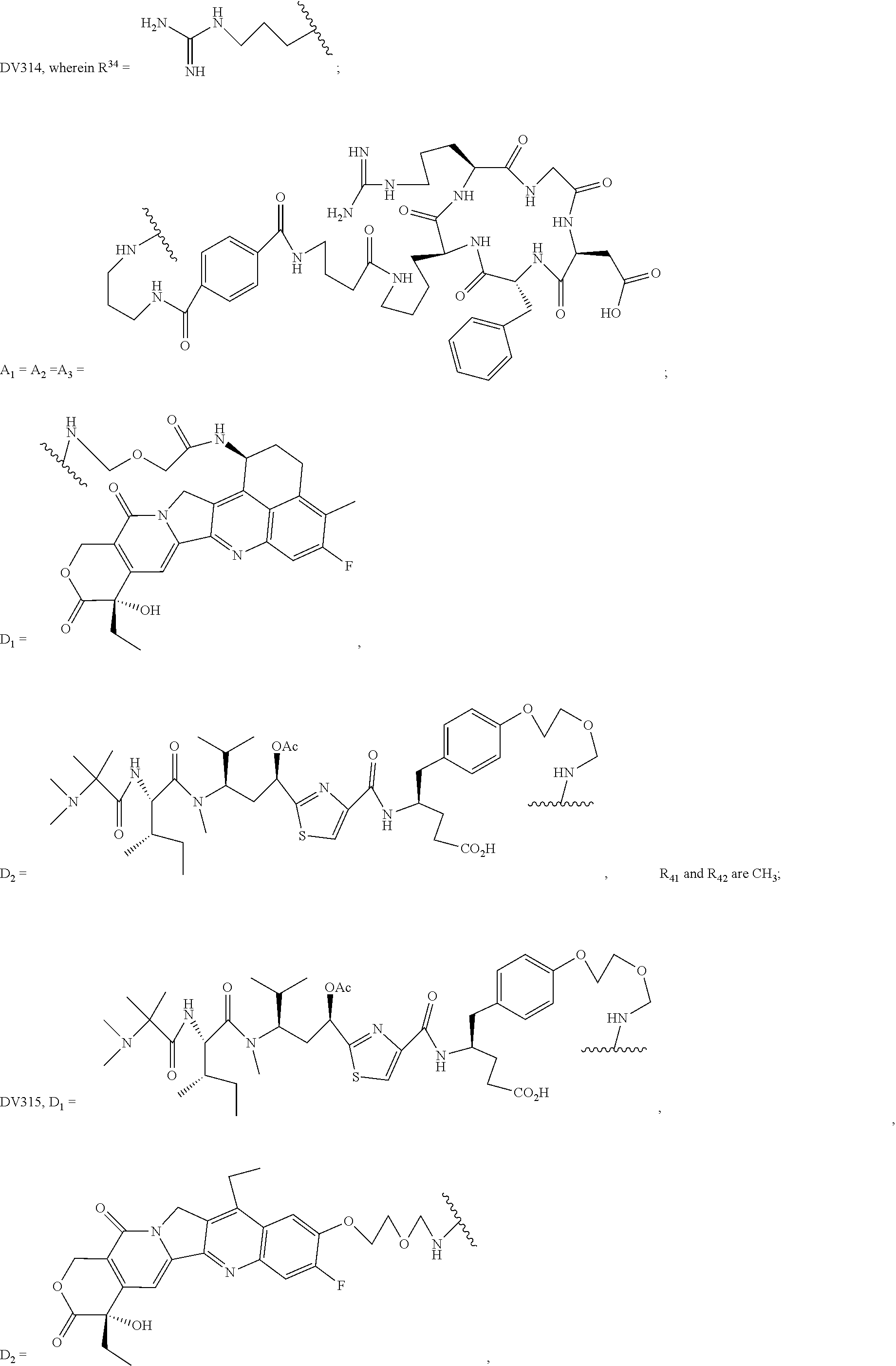

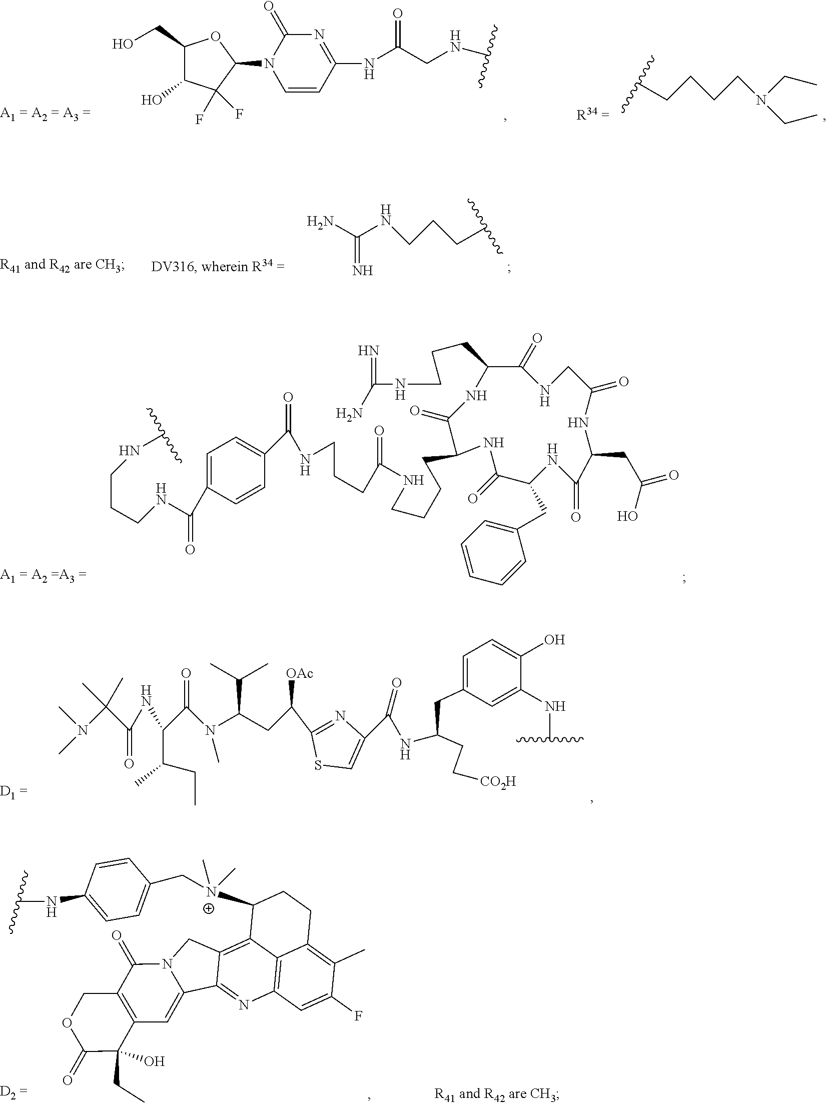

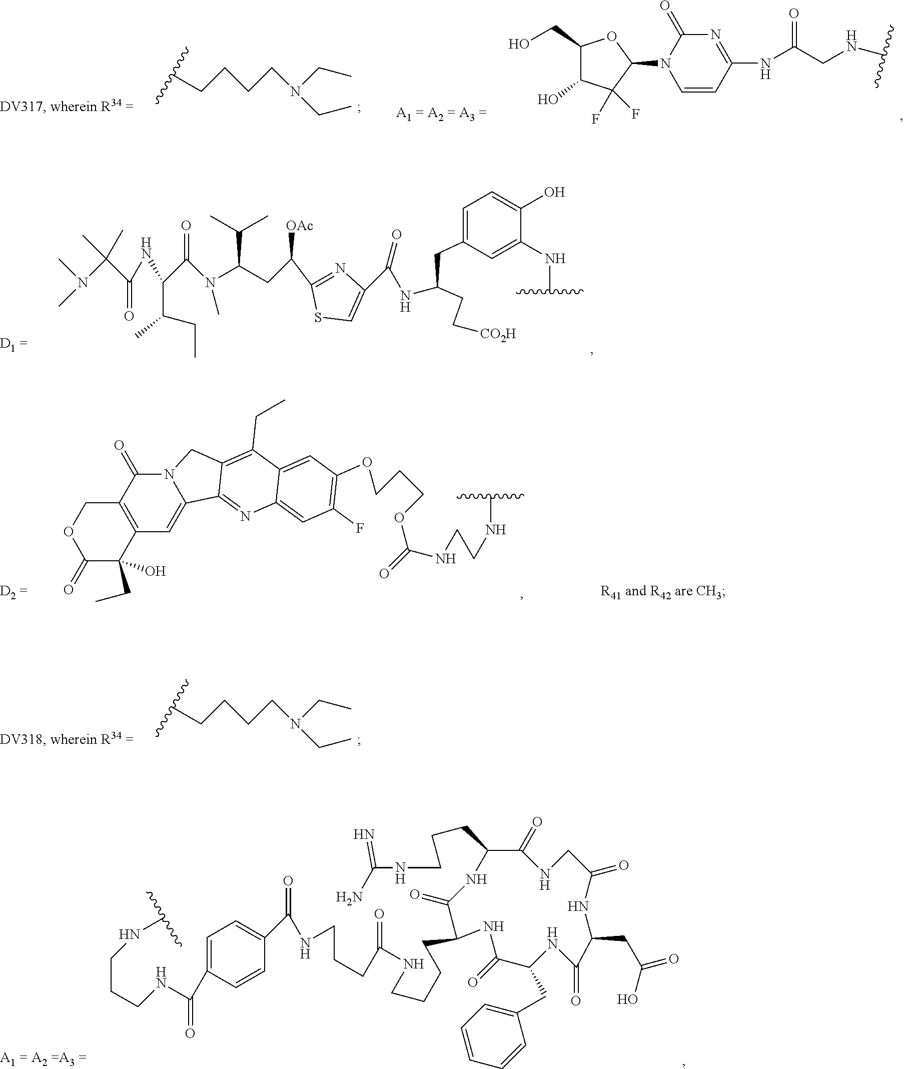

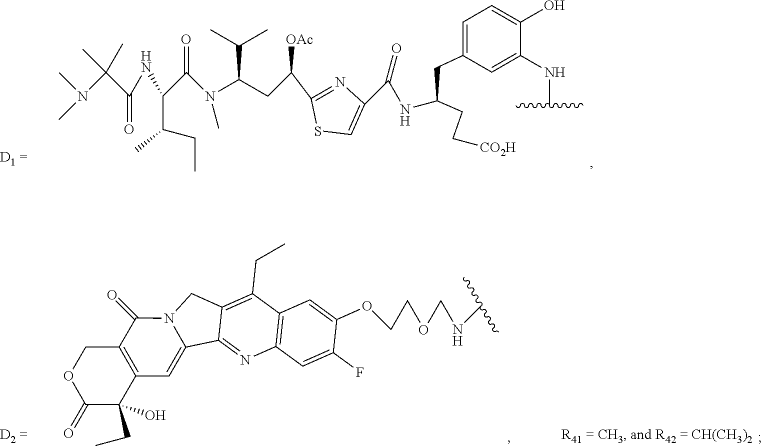 [SMILES: *Nc1cc(C[C@@H](CCC(=O)O)NC(=O)c2csc([C@@H](C[C@H](C(C)C)N(C)C(=O)[C@@H](NC(=O)C(C)(C)N(C)C)[C@@H](C)CC)OC(C)=O)n2)ccc1O ; *NCOCCOc1cc2c(CC)c3c(nc2cc1F)-c1cc2c(c(=O)n1C3)COC(=O)[C@]2(O)CC]

In some embodiments, the antibody drug conjugates are preferably prepared via a homogenous conjugation process, which comprises the following three key steps:
(a) incubating the antibody in the presence of an effective zinc cation-amino chelate/complex (Zn(NR₁R₂R₃)ₘ₁²) and a reductant (e.g. Tris(2-carboxyethyl)phosphine (TCEP)) in a buffer system (e. g. PBS, Mes, Bis-Tris, Bis-Tris Propane, Pipes, Aces,Mopso, Bes, Mops, Hepes, Tes, Pipps, Dipso, Tapso, Heppso, Tris-up, Tris-HCl, Tricine, Hepps, Gly-Gly, Bicine, Taps, Hepee, Acetates, Histidine, Citrates, MES, orBorates, etc.) at pH 4.5 ~8.5, 1-10° C. for 1 ~24 h to selectively reduce interchain disulfide bonds within the antibody, to generate thiols;
(b). introducing an effective amount of a cytotoxic drug-linker complex of formula (V), (VI) (VII) or (V′), bearing thiol reactive groups (e.g., a drug containing maleimide terminal) to react with the thiol groups resulted from step (a); and
(c). adding an effective amount of oxidant (e. g. dehydroascorbic acid (DHAA)) to re-oxidize unreacted thiol groups and then purifying the resulted conjugates;
(d). the step (c) can be replaced by: adding an effective amount of cystine to quench the excessive conjugation linker or linker/payload complex containing thiol reactive groups (e. g. maleimide); and simultaneously or sequentially adding an azido compound (e. g. 4-(azidomethyl)-benzoic acid) or a disulfide compound (e. g. cystine) to quench the unreacted reductant (e. g. TCEP or Tris(hydroxypropyl)phosphine). The addition of cystine to co. quench the unreacted reductant (e. g. TCEP) can form a cysteine which can simultaneously quench the excessive conjugation linker or linker/payload complex of formula (V), (VI), (VII) or (VIII), containing thiol reactive groups (e. g. maleimide).
wherein R₁, R₂ and R₃ in the formula of Zn(NR₁R₂R₃)ₘ²⁺ are independently selected from C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; m₁ is selected from 1, 2, 3, 4, 5, 6, 7 or 8; Preferably m₁ is 1, 2, 3 or 4.

In addition, (NR₁R₂R₃)ₘ₁ can be form a dimer, trimer, tetramer, pentamer, or hexamer wherein these polymers are covalently linked among N, R₁, R₂ and R₃; and N, R₁, R₂ or R₃ themselves or jointly (together) can form heterocyclic, carbocyclic, diheterocyclic, or dicarbocyclic rings.

The Zinc cation-amino chelate/complex, Zn (NR₁R₂R₃)ₘ₁₂+, used in step (a) is 0.01 mM -1.0 mM in concentration, or 0.5 ~20 equivalents in moles of the protein used, and it can be added to the reaction solution with a water-soluble organic solvent, selected from, ethanol, methanol, propanol, propandiol, DMA, DMF, DMSO, THF, CH₃CN.

The reductant is an organic phosphine, preferably selected from Tris(2-carboxyethyl)-phosphine (TECP) or Tris(hydroxypropyl)phosphine and its use in the reaction solution is 0.02 mM-1.0 mM in concentration, or 1.0-20 equivalents in moles of the protein used. The oxidant to be added in step (c) may be DHAA, Fe³⁺, I₂, Cu²⁺, Mn³⁺, MnO₂, or mixture of Fe³⁺/I⁻. The oxidant used in the reaction solution is 0.02 mM -1.0 mM in concentration, or 0.2 -100 equivalents in moles of the protein used. The optimum pH in the conjugation reaction is typically between about 5.0 to 8.0, and preferably, about 5.5 to 7.5. The optimum temperature in the conjugation reaction is typically between about - 5 to about 40° C., and preferably, about 0 to 37° C.; more preferably about 2 to 8° C.; further preferably about 2 to 6° C. The optimum time of the conjugation reaction is typically between about 1 5 m i n to about 48 hours and preferably, about 30 min to overnight (10 ~16 h), more preferably about 2 h ~6 h. The optimal reaction conditions (e. g. pH, temperature, buffer, concentrations of the reactants) of course are depended upon specifically an antibody-like protein, a payload/linker complex, a reductant and/or Zn(NR₁R₂R₃)ₘ₁₂+used.

In further embodiments, Zn(NR₁R₂R₃)ₘ₁²⁺ is preferably selected from: Zn(NH₂CH₃)₂²⁺Zn(NH₂CH₂CH₃)₂²⁻, Zn(NH₂CH₂CH₂CH₃)₂²⁻, Zn(NH₂CH(CH₃)₂)₂²⁺, Zn(NH₂C(CH₃)₃)₂²⁺, Zn(NH₂CH₂C(CH₃)₃)₂²⁻, Zn(NH(CH₃)₂)₂²⁺, Zn(NH(CH₂CH₃)₂)₂²⁺, Zn(NH(CH(CH₃)₂)₂)₂²⁺, Zn(NH(C(CH₃)₃)₂)₂²⁺, Zn(NH(CH(CH₂CH₃)₂)₂)₂²⁺, Zn(NH(CH₂C(CH₃)₃)₂)₂²⁻, Zn(NH(CH₂C(CH₂CH₃)₃)₂)₂²⁺, Zn(NH(CH₂CH₂C(CH₃)₃)₂)₂²⁻, Zn(NH₂CH₂CH₂OH)₂²⁻, Zn(NH(CH₂CH₂OH)₂)₂²⁺, Zn(N(CH₂CH₂OH)₃)₂²⁻, Zn(NH₂CH₂COOH)₂²⁺, Zn(NH₂CH₂CONH₂)₂²⁺, Zn(NH₂CH₂COOCH₃)₂²⁺, Zn(NH₂CH₂COOCH₂CH₃)₂²⁻, Zn(NH₂CH₂COOC(CH₃)₃)₂²⁻, Zn(NH₂CH₂COOCH(CH₃)₂)₂²⁻, Zn(NH₂CH₂CH₂COOH)₂²⁺, Zn(NH(CH₂COOH)₂)₂²⁺, Zn(N(CH₂CH₂COOH)₃)₂²⁻, Zn(NH₂CH₃)₄²⁺, Zn(NH₂CH₂CH₃)₄²⁺, Zn(NH₂CH₂CH₂CH₃)₄²⁺, Zn(NH₂CH(CH₃)₂)₄²⁺, Zn(NH₂C(CH₃)₃)₄²⁺, Zn(NH₂CH₂C(CH₃)₃)₄²⁺, Zn(NH(CH₃)₂)₄²⁺, Zn(NH(CH₂CH₃)₂)₄²⁺, Zn(NH(CH(CH₃)₂)₂)₄²⁺, Zn(NH(C(CH₃)₃)₂)₄²⁺, Zn(NH(CH(CH₂CH₃)₂)₂)₄²⁺, Zn(NH(CH₂C(CH₃)₃)₂)₄²⁺, Zn(NH(CH₂C(CH₂CH₃)₃)₂)₄²⁺, Zn(NH(CH₂CH₂C(CH₃)₃)₂)₄²⁺, Zn(NH₂CH₂CH₂OH)₄²⁺, Zn(NH(CH₂CH₂OH)₂)₄²⁺, Zn(N(CH₂CH₂OH)₃)₄²⁺, Zn(NH₂CH₂COOH)₄²⁺Zn(NH₂CH₂CONH₂)₄²⁺, Zn(NH₂CH₂COOCH₃)₄²⁺, Zn(NH₂CH₂COOCH₂CH₃)₄²⁺, Zn(NH₂CH₂COOC(CH₃)₃)₄²⁺, Zn(NH₂CH₂COOCH(CH₃)₂)₄²⁺, Zn(NH₂CH₂CH₂COOH)₄²⁺Zn(NH(CH₂COOH)₂)₄²⁺, Zn(N(CH₂CH₂COOH)₃)₄²⁺

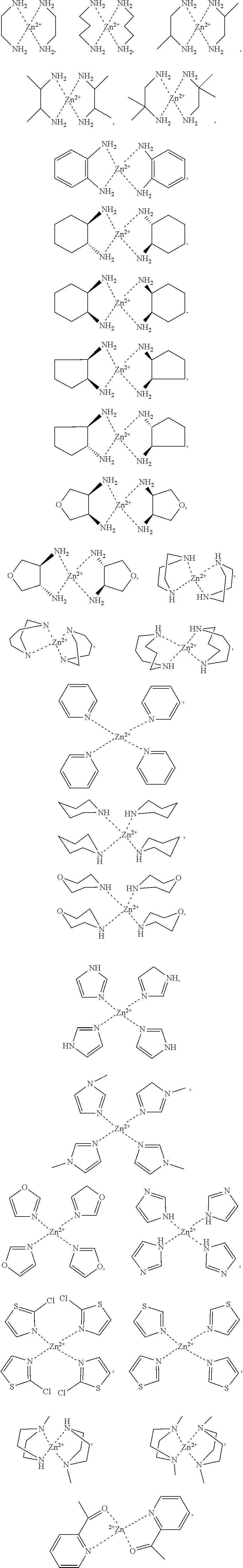

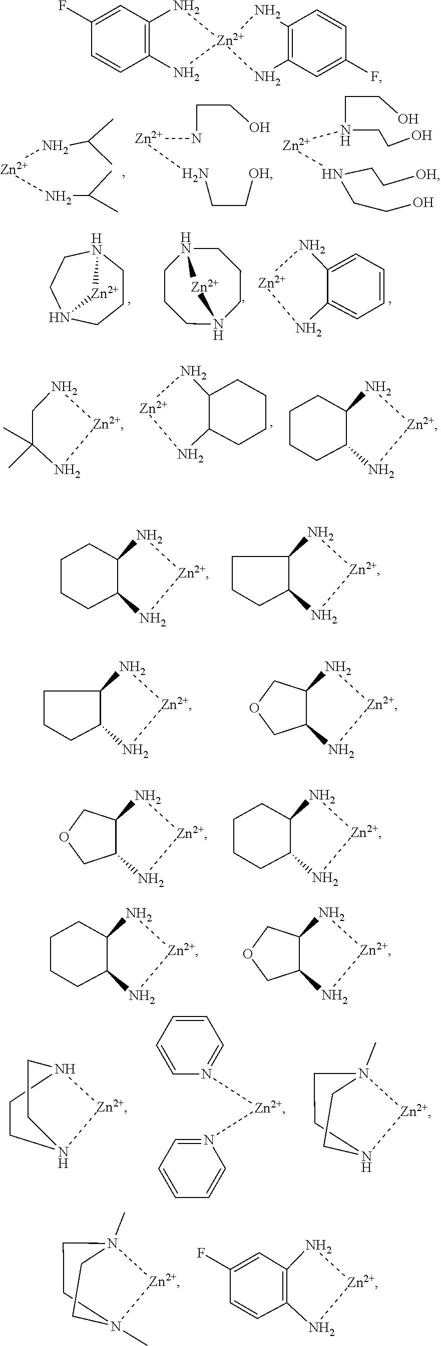

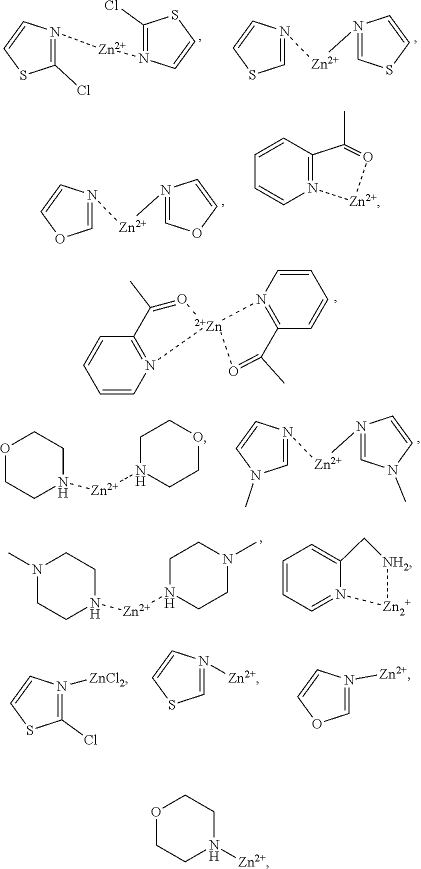

All the complex cations above can be formed with an anion, selected from, but not limited, Cl⁻, Br⁻, I⁻, SO₄²−, HSO₄⁻, NO₃⁻, PO₄³⁻, HPO₄²⁻, H₂PO₄⁻, CO₃²⁻, HCO₃⁻, HCOO⁻, CH₃COO⁻, F₃CCOO⁻, Cl₃CCOO⁻, FCH₂COO⁻, ClCH₂COO⁻, F₂CHCOO⁻, Cl₂CHCOO⁻, BF₄⁻, SO₃²⁻, HSO₃⁻, CH₃SO₃⁻, C₆H₅CH₂SO₃⁻, C₆H₅SO₃⁻, C₆H₅COO⁻, C₆H₅CH₂COO⁻, C₆F₅O⁻, C₆H₄(OH)COO⁻, C₆H₂F₃O⁻, C₆ H₄(NO₂)O⁻, C₆ H₂(NO₂)₃⁻, etc.

In further embodiments, under the homogenous conjugation process, the resulted conjugates of formula (I), (II), (III) or (IV) are over 75% linked to the cysteine sites between heavy-light chains of an antibody, and are less than 15% linked to the cysteine sites between heavy-heavy chains (hinge region) of an antibody. Typically, for formula (I), (II) (III) or (IV), when drug/antibody ratio (DAR) is set to be 4 and a drug linked to the linker is at one-to-one ratio, the distributions in percentage of the numbers of drugs in the antibody are: D0<1%, D2<10%, D4>65%, D6<10%, D8<10%; for formula (III). If the drug to linker ratio is above to 1, such as, 2, 3, 4, 5, or 6 drugs per linker (when a side chain or a multiple branched linker is used), the distributions in percentages of numbers of drugs in the antibody or antibody-like protein are increased by timing the ratios of drug/linker accordingly. The DAR can also be set up to around 6, with majority D6 >65%, using both more equivalents of reducing agents, such as TCEP and more equivalents of one drug per linker payload/linker complex of formula (V), (VI), (VII) and (V′), wherein the drugs are mainly conjugated to the sites of disulfide bonds between heavy-light chains and the disulfide bonds of the upper hinge region of IgG antibodies.

The resulted conjugates may be purified by standard biochemical means, such as gel filtration on a Sephadex G25 or Sephacryl S300 column, adsorption chromatography, ion (cation or anion) exchange chromatography, affinity chromatography (e.g. protein A column) or by dialysis (ultrafiltration or hyperfiltration (UF) and diafiltration (DF)). In some cases, a small size molecule of antibody (e. g. <100 KD) conjugated with a small molecular drug can be purified by a chromatography such as by (reverse phase) HPLC or FPLC, size-exclusion chromatography, medium pressure column chromatography, ion exchange chromatography, or hydroxyapatite chromatography.

In further embodiments, for preparation of thr conjugate of Formula (IV), the reaction of the cytotoxic drug/cytotoxic drug-linker complex of Formula (V) and (V′) to an amino acid in the antibody can be conducted at simultaneously or sequentially at the same or different conditions in the same pot. If the conjugation reactions are preformed simultaneously at the same condition, the Lv₁ of Formula (V) and Lv₂ of formula (V′) are normally differentiated. For instance, a thiol reactive group (e. g. maleimido, vinylsulfonyl, haloacetyl, acrylic, substituted propiolic) is selected for Lv₁, then an amino reactive group of N-hydroxysuccinimidyl (NHS) ester, pentafluorophenyl ester, dinitrophenyl ester, or carboxylic acid chloride group can be chosen for Lv₂. A clickable chemistry group (e. g. azide, alkyne, dibenzocyclooctyne, BCN ((1R, 8S, 9s)-bicyclo[6.1.0]non-4-yn-9-ylmethanol)) can be selected for either Lv₁ or Lv₂ if the conjugated antibody is introduced a clickable reactive group ahead of the click chemistry reaction. Lv₁ and Lv₂ can be selected from many pairs of different function/reactive groups, such as: Amine-to-Sulfhydryl (succinimidyl (NHS) ester/maleimide, NHS ester/ pyridyldithiol, NHS esters/haloacetyl), diazirine (SDA)-to-Sulfhydryl, Azide-to-Sulfhydryl, Alkyne-to-Sulfhydryl, Sulfhydryl-to-Carbohydrate (Maleimide/Hydrazide, Pyridyldithiol/Hydrazide, haloacetyl/Hydrazide), Hydroxyl-to-Sulfhydryl (Isocyanate/Maleimide), Sulfhydryl-to-DNA (Maleimide/Psoralen, Pyridyldithiol/ Psoralen, haloacetyl/Psoralen), Sulfhydryl-to-Carboxyl (Carbodiimide). The conjugation reactions of Formula (V) and (V′) can also be performed sequentially, as long as Lv₁ and Lv₂ have different reactive ability to the amino acids in the antibody /antibody like protein. For example, when both Lv₁ and Lv₂ are selected for reacting to thiols (cysteines) in the antibody/protein, Lv₁ can be maleimido, which can react to a thiol group as fast as a few seconds at pH 5,0~7.5, 2.0 ~37° C.; Lv₂ is then selected from slow reactive vinylsulfonyl or haloacetyl group in which the conjugation reaction with a thiol group in antibody has to be at pH >7.0, temperature over 30° C. for over 6 h.

If both Lv₁ and Lv₂ have the same (terminal) conjugatable groups, e. g., maleimido, haloacetyl or pyridyldithiol, the first conjugation with Formula (V) can be performed according to the above homogenous conjugation process, wherein the payload/linker complex is conjugated to the disulfide sites between heavy-light chains (of the Fab region) of a IgG antibody, then the Formula (V′) is adding sequentially to the reaction mixture to be conjugated at the disulfide bonds of hinge region of the IgG antibody. The first step of conjugation reaction can be conducted at low temperature, and the second step of the conjugation reaction with Formula (V′) can then be performed at room (25° C.) or higher temperature without purification of the first step reaction product as long as the compound of Formula (V′) is added in much more (3 or more) equivalents then the compound of Formula (V). When the same pH and/or temperature conditions are chosen for thioether linked conjugates under the above homogenous conjugation process, the over four times equivalents of the cytotoxic drug-linker complex containing dual terminal thiol reactive are used for the cross conjugation of disulfide bonds of heavy-light chains of an antibody. It should be noted that a preferred method of synthesis of the disulfide or thiol-ether linked conjugates can be through the first chemical synthesis the drug-linker complex having disulfide or thiol-reactive compounds of the formula (V), (VI), (VII) or (V′); following by reaction with the thiols in the protein (antibody) according the process of the invention. Synthesis of conjugates bearing an acid labile hydrazone linkage can be achieved by reaction of a carbonyl group with the hydrazide moiety in the linker, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997). Synthesis of conjugates bearing triazole linkage can be achieved by reaction of a 1-yne group of the cytotoxic drug/cytotoxic drug-linker complex or a binding ligand/binding ligand-linker complex with the azido moiety in the linker of formula (V), (VI), (VII), or (V′), through the click chemistry (Huisgen cycloaddition) (Lutz, J-F. et al, 2008, Adv. Drug Del. Rev. 60, 958-70; Sletten, E. M. et al 2011, AccChem. Research 44, 666-76). Synthesis of the conjugates linked via oxime is achieved by reaction of a modified antibody containing a ketone or aldehyde and a cytotoxic drug/cytotoxic drug-linker complex or a binding ligand/binding ligand-linker complex containing oxylamino group. A cytotoxic drug/cytotoxic drug-, or a binding ligand/binding ligand-linker complex containing an amino group can condensate with a carboxyl ester of NHS, imidazole, nitrophenoxyl; N-hydroxysuccinimide (NHS); methylsulfonyl-phenoxyl; dinitrophenoxyl; pentafluorophenoxyl; tetrafluorophenoxyl; difluorophenoxyl; monofluo-rophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate in an antibody-linker complex to give a conjugate via amide bond linkage of Formula (I), (II), (III) or (IV). Many regular chemical and biochemical processes of the antibody-drug conjugation are known in the art (see, e. g. Matsuda, Y. and Mendelsohn, B. A., Expert Opin Biol Ther. 2021, 21(7): 963-975; Puthenveetil, S., Methods Mol Biol. 2020, 2078: 99-112; van Delft, F., and Lambert, J. M., ed. “Chemical Linkers in Antibody-Drug Conjugates (ADCs)”, Royal Soc. Chem. Pub., 22, Dec. 2021, ISBN 978-1-83916-263-3, doi:10.1039/9781839165153; Tumey, L. N., ed. “Antibody-Drug Conjugates, Methods and Protocols”, Springer Pub., 2020, ISBN: 978-1-4939-9929-3; Khongorzul, P. et al, Mol Cancer Res. 2020,18(1):3-19; and many references incorporated in these books and papers).

In over all, it is more preferable that the conjugates of Formula (I), (II), (III) and (IV) are directly conjugated with Formula (V), (VI), (VII), and (V) plus (V′) accordingly to an antibody in water-based solution, and the compounds of Formula (V), (VI), (VII) and (V′) are constructed by chemical synthesis.

In general, the conjugate of Formula (I), (II), (III) or (IV) is preferably generated from a drug/linker complex of Formula (V), (VI), (VII), or (V) plus (V′), as in a one pot reaction. When a thiol reduced from an antibody reacts a thiol reactive group in the terminal of drug/linker complex of Formula (V), (VI), (VII), or (V′), the Ellman reagent can be optionally used to monitor the efficient reduction of the disulfide bonds and conjugation of the thiols through measurement of the numbers of the free thiols during the reactions. A UV spectrometry at wavelength of range 190-390 nm, preferably at 240-380 nm, more preferably at 240-370 nm is preferred to be used in assisting the reaction (via monitoring the conjugation). The conjugation reaction can be thus measured or conducted in a quartz cell or Pyrex flask in temperature control environment. The drug/protein (antibody) ratios (DAR) of the conjugates can also be measured by UV at wavelength of range 240-380 nm via calculation of the concentrations of the drug and the protein, by Hydrophobic Interaction Chromatography (HIC-HPLC) or Reverse Phase Chromatography (RP-HPLC) via measurement of the integration areas of each drug/protein fragment, or by Capillary electrophoresis (CE), and/or by LC-MS or LC-MS/MS or CE-MS (the combination of liquid chromatography (LC) or CE with mass spectrometry (MS) via measurement of both the integration areas of LC or CE and Peak intensity of MS for each drug/protein fragment). It is also noted in the conjugation process of the present invention, when a drug or a drug/linker complex is not well soluble in a water-based buffer solution, up to 30% of water mixable (miscible) organic solvents, such as DMA, DMF, ethanol, methanol, acetone, acetonitrile, THF, isopropanol, dioxane, propylene glycol, or ethylene diol can be added as the co-solvent in water-based buffer solution.

The aqueous solutions for the modification of the antibody are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, sucrose and salts, for examples, NaCl and KCl. Other biological buffers that are used for the conjugation process are listed in the definition section. The progress of the reaction can be monitored by measuring the decrease in the absorption at a certain UV wavelength, such as at 254 nm, or increase in the absorption at a certain UV wavelength, such as 280 nm, or the other appropriate wavelength. After the reaction is complete, isolation of the modified cell-binding antibody agent can be performed in a routine way, using for example gel filtration chromatography, or adsorptive chromatography. When disulfide exchange reaction is used for modification of the antibody, the extent of the modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by UPLC-QTOF mass spectrometry, or Capillary electrophoresis-mass spectrometry (CE-MS). The linker compounds have diverse functional groups that can react with drugs, preferably cytotoxic agents that possess a suitable substituent. For examples, the modified antibody bearing an amino or hydroxyl substituent can react with drugs bearing an N-hydroxysuccinimide (NHS) ester, the modified antibody bearing a thiol substituent can react with drugs bearing a maleimido or haloacetyl group. Additionally, the modified antibody bearing a carbonyl (ketone or aldehyde) substituent can react with drugs bearing a hydrazide or an alkoxyamine. One skilled in the art can readily determine which linker to use based on the known reactivity of the available functional group on the linkers.

### Formulation and Application

The antibody drug conjugates of the patent application are formulated to liquid, or suitable to be lyophilized and subsequently be reconstituted to a liquid formulation. The conjugate in a liquid formula or in the formulated lyophilized powder may take up 0.01%-99% by weight as major gradient in the formulation. In general, a liquid formulation comprising 0.1 g/L~300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as sodium, potassium, ammonium, or trihydroxyethylamine salts of citric acid, ascorbic acid, gluconic acid, carbonic acid, tartaric acid, succinic acid, acetic acid or phthalic acid; Tris, tromethamine hydrochloride, sulfate or phosphate buffer. In addition, amino acid cationic components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-arginyl succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. The polyol in the liquid formula or in the formulated lyophilized solid can be 0.0% -20% by weight. Preferably, a nonreducing sugar, sucrose or trehalose at a concentration of about from 0.1% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc); etc. Preferred surfactants are polyoxyethylene sorbitan fatty acid esters e.g. polysorbate 20, 40, 60 or 80 (Tween 20, 40, 60 or 80).

The concentration of a surfactant in the formulation is range from 0.0% to about 2.0% by weight. In certain embodiments, the surfactant concentration is from about 0.01% to about 0.2%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenoxyl, butyl and benzyl alcohol, alkyl parabens such as methyl or propyl paraben, catechol, resorcinol, cyclohexanol, 3-pentanol, and m-cresol. The preservative in the liquid formula or in the formulated lyophilized powder can be 0.0% -5.0% by weight. In one embodiment, the preservative herein is benzyl alcohol.

Suitable free amino acids as a bulky material, or tonicity agent, or osmotic pressure adjustment in the formulation, is selected from, but are not limited to, one or more of arginine, cystine, glycine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine, then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The amino acid in the liquid formula or in the formulated lyophilized powder can be 0.0% -30% by weight.

The formulations can optionally comprise methionine, glutathione, cysteine, cystine or ascorbic acid as an antioxidant at a concentration of about up to 5 mg/ml in the liquid formula or 0.0%-5.0% by weight in the formulated lyophilized powder; The formulations can optionally comprise metal chelating agent, e.g., EDTA, EGTA, etc., at a concentration of about up to 2 mM in the liquid formula or 0.0%-0.3% by weight in the formulated lyophilized powder.

The final formulation can be adjusted to the preferred pH with a buffer adjusting agent (e.g. an acid, such as HCl, H₂SO₄, acetic acid, H₃PO₄, citric acid, etc, or a base, such as NaOH, KOH, NH₄OH, ethanolamine, diethanolamine or triethanol amine, sodium phosphate, potassium phosphate, trisodium citrate, tromethamine, etc) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. The isotonic agent is selected from mannitol, sorbitol, sodium acetate, potassium chloride, sodium phosphate, potassium phosphate, trisodium citrate, or NaCl. In general, both the buffer salts and the isotonic agent may take up to 30% by weight in the formulation.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, (2-Hydroxypropyl)-$-cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenoxyl, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: The Science and Practice of Pharmacy, 21^{th} edition, Gennaro, Ed., Lippincott Williams & Wilkins (2005).

A pharmaceutical container or vessel is used to hold the pharmaceutical formulation of any of conjugates of the patent application. The vessel is a vial, bottle, pre-filled syringe, pre-filled or auto-injector syringe. The liquid formula can be freeze-dried or drum-dryed to a form of cake or powder in a borosilicate vial or soda lime glass vial. The solid powder can also be prepared by efficient spray drying, and then packed to a vial or a pharmaceutical container for storage and distribution.

In a further embodiment, the invention provides a method for preparing a formulation comprising the steps of: (a) lyophilizing the formulation comprising the conjugates, excipients, and a buffer system; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media, several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as the antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or lyophilizate formulation should exhibit a shelf life of more than 6 months at 25° C. More preferred a conjugate in either liquid or lyophilizate formulation should exhibit a shelf life of more than 12 months at 25° C. Most preferred liquid formulation should exhibit a shelf life of about 24 to 36 months at 2-8° C. and the lyophilizate formulation should exhibit a shelf life of about preferably up to 60 months at 2-8° C. Both liquid and lyophilizate formulations should exhibit a shelf life for at least two years at −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody ratio and aggregate formation (for example using UV, size exclusion chromatography, by measuring turbidity, and/or by visual inspection); by assessing charge heterogeneity using cation exchange chromatography, image capillary isoelectric focusing (iCIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS-—C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomerization), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 24 months, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8~108 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4 ~108 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 25 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

In some embodiment, when the reconstituted conjugates are injected under the skin, into a muscle, or into other tissues of the body, a hyaluronidase (HAase) is preferably administered together with the conjugates. The hyaluronidase here is used as an aid in helping patient body absorb the injected conjugates. The hyaluronidase is synergistically used 20-200-unit doses, preferably in 40 -160-unit doses.

The conjugates of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/triweekly/monthly dose ranges are from 0.01 to 100 mg/kg of body weight. By way of general guidance, unit doses for humans range from 1 mg to 3000 mg per day, or per week, per two weeks (biweekly), triweekly, or per month.

Preferably the unit dose range is from 1 to 500 mg administered one to four times a month and even more preferably from 1 mg to 100 mg, once a week, or once a biweek, or once a triweek. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasally, particularly in the form of powders, nasal drops, or aerosols; or dermally, for example, topically in ointments, creams, lotions, gels or sprays, or via trans-dermal patches. The compositions may conveniently be administered in unit dosage form and may be prepared by any of the methods well known in the pharmaceutical art, for example, as described in Remington: The Science and Practice of Pharmacy, 21^{th} ed.; Lippincott Williams & Wilkins: Philadelphia, PA, 2005.

The formulations include pharmaceutical compositions in which a compound of the present invention is formulated for oral or parenteral administration. For oral administration, tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal. Preferred tablets contain lactose, cornstarch, magnesium silicate, croscarmellose sodium, povidone, magnesium stearate, or talc in any combination.

Liquid preparations for parenteral administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

In yet another embodiment, a pharmaceutical composition comprising a therapeutically effective amount of the conjugate of Formula (I), (II), (III), (IV) or any conjugates described through the present patent can be coadministered with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The term “coadministered,” as used herein, refers to administering one or more additional therapeutic agents and the antibody or ADC described herein, or the antibody or ADC-containing composition, sufficiently close in time such that the antibody or ADC can enhance the effect of one or more additional therapeutic agents, or vice versa. In this regard, the antibody or ADC or the composition containing the same may be administered first, and the one or more additional therapeutic agents may be administered second, or vice versa. For example, the antibody or ADC or composition containing the same may be administered in combination with other agents (e.g., as an adjuvant) for the treatment or prevention of multiple myeloma. In this respect, the antibody or ADC or antibody or ADC-containing composition can be used in combination with at least one other anticancer agent including, for example, any suitable chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer), and/or surgery. The synergistic drugs or radiation therapy can be administered prior or subsequent to administration of a conjugate, in one aspect at least an hour, 12 hours, a day, a week, biweeks, triweeks, a month, in further aspects several months, prior or subsequent to administration of a conjugate of the invention.

The synergistic agents are preferably selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-lb, Interferon alfa-2a, Interferon alfa-2b, Interferon beta, Interferon beta la, Interferon beta Ib, Interferon gamma-Ia, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, R₁₀uximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.

In some embodiments, the disclosure also provides a composition comprising the above-described antibody or antibody-drug conjugate and a pharmaceutically acceptable (e.g., physiologically acceptable) carrier. Any suitable carrier known in the art can be used within the context of the invention. The choice of carrier will be determined, in part, by the particular site to which the composition may be administered and the particular method used to administer the composition. The composition optionally may be sterile. The compositions can be generated in accordance with conventional techniques described in, e.g., Remington: The Science and Practice of Pharmacy, 21st Edition, Lippincott Williams & Wilkins, Philadelphia, Pa. (2001).

The composition of this invention desirably comprises the antibody or ADCs in an amount that is effective to treat or prevent cancers. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the antibody or ADC or the composition comprising the antibody or ADC and a pharmaceutically acceptable carrier. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the antibody or ADC to elicit a desired response in the individual. For example, a therapeutically effective amount of the ADC of the invention is an amount which binds to a certain antigen on cancer cells and destroys them.

A pharmacologic and/or physiologic effect of treatment may be prophylactic, i.e., the effect completely or partially prevents a disease or symptom thereof. In this respect, the inventive method comprises administering a “prophylactically effective amount” of the ADC or a composition comprising the ADC to a mammal that is predisposed to multiple myeloma. A “prophylactically effective amount” refers to an amount effective, at dosages and for periods of time necessary, to achieve a desired prophylactic result (e.g., prevention of disease onset). Therapeutic or prophylactic efficacy can be monitored by periodic assessment of treated patients. In one embodiment, the ADC described herein inhibits or suppresses proliferation of prostate cancer cells by at least about 10% (e.g., at least about 20%, at least about 30%, at least about 40%, at least about 50%, at least about 60%, at least about 70%, at least about 80%, at least about 90%, or at least about 100%). Cell proliferation can be measured using any suitable method known in the art, such as measuring incorporation of labeled nucleosides (e.g., 3H-thymidine or bromodeoxyuridine Brd(U)) into genomic DNA (see, e.g., Madhavan, H. N., J. Stem Cells Regen. Med., 3(1): 12-14 (2007)).

The invention of the ADCs further provides a method of treating a patient having or at risk of having an immune disorder mediated by immune cells expressing the antigens comprising administering to the patient an effective regime of any of the above-described ADCs. Optionally, the disorder is a B cell mediated disorder. Optionally, the immune disorder is rheumatoid arthritis, systemic lupus E (SLE), Type I diabetes, asthma, atopic dermitus, allergic rhinitis, thrombocytopenic purpura, multiple sclerosis, psoriasis, Sjorgren's syndrome, Hashimoto's thyroiditis, Grave's disease, primary biliary cirrhosis, Wegener's granulomatosis, tuberculosis, and graft versus host disease.

In some embodiments, the invention of the ADCs further provides a method of treating a patient having or at risk of having a cancer, an autoimmune disease, an infectious disease, viral disease or a pathogenic infection, through administering to the patient an effective regime of any of the above ADCs, or any of the above described ADCs concurrently with the other therapeutic agents such as the chemotherapeutic agent, the radiation therapy, immunotherapy agents, autoimmune disorder agents, anti-infectious agents or the other conjugates.

The targeted cancer includes, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Mycloid, Chronic Lymphocytic, Chronic Myclogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

The autoimmune disease includes, but are not limited, Achlorhydra Autoimmune Active Chronic Hepatitis, Acute Disseminated Encephalomyclitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barr6 syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, M6niere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sj5gren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome.

The infectious disease includes, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Striussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The pathogenic strain includes, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4) -Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157:H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

The pathogenic viruses, includes, but not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the ADCs of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 μM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples further illustrate the invention but, of course, should not be construed as in any way limiting its scope.

### EXAMPLES

The invention is further described in the following examples, which are not intended to limit the scope of the invention. Cell lines described in the following examples were maintained in culture according to the conditions specified by the American Type Culture Collection (ATCC), Deutsche Sammlung von Mikroorganismen und Zellkulturen GmbH, Braunschweig, Germany (DMSZ), The Shanghai Cell Culture Institute of Chinese Acadmy of Science, or Nanjing Cobioer Biosciences Co., unless otherwise specified. Cell culture reagents were obtained from Invitrogen Corp., unless otherwise specified. All anhydrous solvents were commercially obtained and stored in Sure-seal bottles under nitrogen. PEG compounds were purchased from Biomatrik Inc, Jiaxing, China. Some chemical compounds, when were not referred synthesis from, were provided by CROs (e. g. Wuxi Apptec, Chemexpress, Raybow Pharma, GL Biochem, Asymchem, and Medicilin (in Hangzhou) in China. Dxd-GGFG payload/linker complex which was used for comparison with the payload/ligand/linker complexes of the present invention was purchased from Chemexpress (Shanghai). Experimental animals were purchased from National Resource Center of Model Mice via GemPharmatech. Co., Ltd, Najing, China and Shanghai SLAC Laboratory Animal Co., Ltd., Shanghai, China. All other reagents and solvents were purchased as the highest grade available and used without further purification. The preparative HPLC separations were performed with Varain PreStar HPLC. HPLC analysis was conducted on Agilent 1260. The mass spectral data were acquired on a Waters Xevo QTOF mass spectrum equipped with Waters Acquity UPLC separations module and Acquity TUV detector. NMR spectra were recorded on Zhongke-niujin WNMR-I 400 MHz instrument at the Department of Chemistry of Zhejiang Sci-Tech University. Chemical shifts (6) are reported in parts per million (ppm) referenced to tetramethylsilane at 0.00 and coupling constants (J) are reported in Hz. The elemental analysis of C, H, and/or N was provided by the Department of Chemistry of Zhejiang Sci-Tech University and conducted on Elementar UNICUBE. Quantitative analysis of metal atoms was performed on Agilent ICPOES 730 ICP-MS.

### Example 1. Synthesis of benzyl -((benzyloxy) carbonyl)-(tert-butoxycarbonyl)-L-lysyl-L-valinate (3)

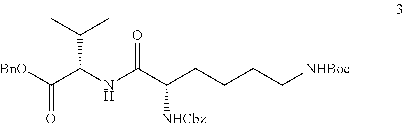 [SMILES: CC(C)[C@H](NC(=O)[C@H](CCCCNC(=O)OC(C)(C)C)NC(=O)OCc1ccccc1)C(=O)OCc1ccccc1]

To a stirred solution of ((benzyloxy) carbonyl)-(tert-butoxycarbonyl)-L-lysine (13.0 g, 34.17 mmol) in DCM (150 mL) at room temperature were added benzyl L-valinate (7.44 g, 35.88 mmol), 2-(7-azabenzotriazol-1-yl)-N, N, N′, N′-tetramethyluronium hexafluorophosphate (19.49 g, 51.25 mmol), and DIEA (8.80 g, 68.84 mmol), the reaction mixture was stirred at room temperature for 2 h, washed with water (200 mL), brine (200 mL), dried over anhydrous sodium sulfate, filtered and concentrated in vacuo to afford the crude product, which was purified by a silica gel column, eluted with DCM and methanol, concentrated to give 17.5 g of white solid (compound 3, 90% yield).

### Example 2. Synthesis of benzyl ((benzyloxy) carbonyl)-L-lysyl-L-valinate (4)

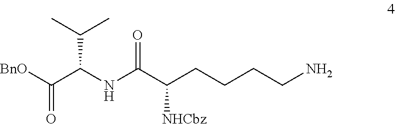 [SMILES: CC(C)[C@H](NC(=O)[C@H](CCCCN)NC(=O)OCc1ccccc1)C(=O)OCc1ccccc1]

To a stirred solution of 3 (5.43 g, 9.53 mmol) in DCM (60 mL) was added TFA (12 mL). The mixture was stirred at room temperature for 2 h, diluted with DCM (200 mL), washed with saturated aqueous NaHCO₃ solution, and brine, dried over anhydrous sodium sulfate and concentrated in vacuo to afford 4.44 g of a white solid (compound 4, 99% yield).

### Example 3 Synthesis of 3-(2-(2-(4-(tert-butoxycarbonyl)benzamido) ethoxy) ethoxy) propanoic acid (7)

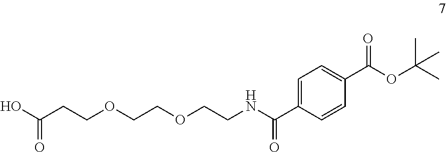 [SMILES: CC(C)(C)OC(=O)c1ccc(C(=O)NCCOCCOCCC(=O)O)cc1]

To a stirred solution of 3-(2-(2-aminoethoxy) ethoxy) propanoic acid (6.6 g, 37.39 mmol) in water (100 mL) at 0° C. were added sodium carbonate (7.9 g, 74.53 mmol), sodium hydroxide (2.9 g, 72.50 mmol), and a solution of tert-butyl 4-(chlor® Carbonyl)benzoate (9.0 g, 37.39 mmol) in THF (40 mL). The reaction mixture was stirred at room temperature for 1 h, quenched with 1 M HCl (100 mL), extracted with DCM (300 mL) for 3 times, the combined organic phases were concentrated to afford the crude, which was purified by a silica gel column, eluted with DCM and methanol, concentrated to afford 10.4 g of a yellow liquid (compound 7, 73% yield).

### Example 4. Synthesis of benzyl (17S, 20S)-17-(((benzyloxy) carbonyl)amino)-1-(4-(tert-butoxycarbonyl)phenyl)-20-isopropyl-1, 11, 18-trioxo-5, 8-dioxa-2, 12, 19-triazahenicosan-21-oate (8)

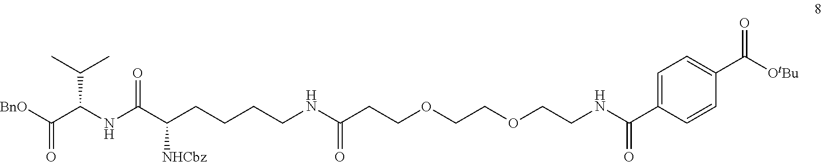 [SMILES: CC(C)[C@H](NC(=O)[C@H](CCCCNC(=O)CCOCCOCCNC(=O)c1ccc(C(=O)OC(C)(C)C)cc1)NC(=O)OCc1ccccc1)C(=O)OCc1ccccc1]

To a stirred solution of 4 (1.66 g, 3.54 mmol) and 7 (1.35 g, 3.54 mmol) in DMF (60 mL) were added HATU (2.03 g, 5.31 mmol) and DIEA (1.37 g, 10.61 mmol). The mixture was stirred at room temperature for 1 h, diluted with DCM (100 mL), washed with water and brine, dried over anhydrous sodium sulfate, and evaporated under reduced pressure. The residue was purified by flash column chromatography (silica gel, 0-10% MeOH in DCM) to afford 1.98 g of a white solid (compound 8, 67% yield).

### Example 5. Synthesis of 4-(((4S, 7S)-7-(((benzyloxy) carbonyl)amino)-4-isopropyl-3, 6, 13-trioxo-1-phenyl-2, 16, 19-trioxa-5, 12-diazahenicosan-21-yl) carbamoyl)benzoic acid (9)

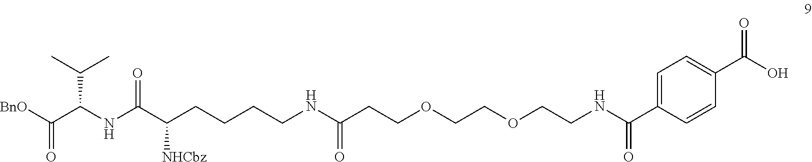 [SMILES: CC(C)[C@H](NC(=O)[C@H](CCCCNC(=O)CCOCCOCCNC(=O)c1ccc(C(=O)O)cc1)NC(=O)OCc1ccccc1)C(=O)OCc1ccccc1]

To a stirred solution of 8 (1.98 g, 2.38 mmol) in DCM (30 mL) was added HCOOH (30 mL). The mixture was stirred at room temperature for 16 h, concentrated in vacuo and the residue was purified by flash column chromatography (silica gel, 0-10% MeOH in DCM) to afford 1.50 g of a yellow solid (compound 9, 81% yield).

### Example 6. Synthesis of 5-benzyl 1-(tert-butyl) (((S)-1, 5-di-tert-butoxy-1, 5-dioxopentan-2-yl) carbamoyl)-L- glutamate (12)

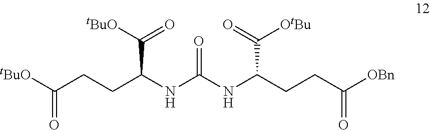 [SMILES: CC(C)(C)OC(=O)CC[C@H](NC(=O)N[C@@H](CCC(=O)OCc1ccccc1)C(=O)OC(C)(C)C)C(=O)OC(C)(C)C]

To a stirred solution of 5-benzyl 1-(tert-butyl) L- glutamate (10.0 g, 33.81 mmol) in DCM (200 mL) at −78° C., under nitrogen were added triphosgene (3.4 g, 11.49 mmol) and TEA (8.2 g, 81.13 mmol). After the resulting mixture was stirred at −78° C. for 2 h, a solution of di-tert-butyl L- glutamate (12.2 g, 37.187 mmol) mixed with TEA (8.2 g, 81.13 mmol) in DCM (60 mL) was added dropwise at −78° C., and the resulting mixture was naturally warmed to room temperature and then stirred overnight, quenched by sodium bicarbonate (100 mL), washed with brine (80 mL), dried over anhydrous sodium sulfate, filtered and concentrated to give the crude, which was purified by a silica gel column, eluted with ethyl acetate and petroleum ether, concentrated to afford 15.0 g of a colorless oil (compound 12, 76% yield).

### Example 7. Synthesis of (S)-5-(tert-butoxy)-4-(3-((S)-1, 5-di-tert-butoxy-1, 5-dioxopentan-2-yl) ureido)-5-oxopentanoic acid (13)

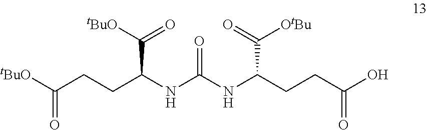 [SMILES: CC(C)(C)OC(=O)CC[C@H](NC(=O)N[C@@H](CCC(=O)O)C(=O)OC(C)(C)C)C(=O)OC(C)(C)C]

To a solution of 12 (15.0 g, 25.92 mmol) in DCM (150 mL) was added Pd/C (1.5 g, 10 wt %), the reaction flask was evacuated and back filled with hydrogen for three times, and then stirred overnight under a hydrogen balloon at 40° C., filtered and concentrated to give 12.9 g of a colorless oil (compound 13, 100% yield).

### Example 8. Synthesis of di-tert-butyl (((S)-1-(tert-butoxy)-5-((2, 5-dioxopyrrolidin-1-yl)oxy)-1, 5-dioxopentan-2-yl) carbamoyl)-L- glutamate (14)

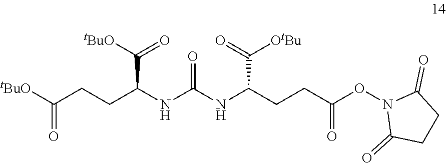 [SMILES: CC(C)(C)OC(=O)CC[C@H](NC(=O)N[C@@H](CCC(=O)ON1C(=O)CCC1=O)C(=O)OC(C)(C)C)C(=O)OC(C)(C)C]

To a stirred solution of 13 (10.0 g, 20.46 mmol) in DCM (150 mL) at room temperature were added N-hydroxysuccinimide (3.5 g, 30.70 mmol) and N-(3-dimethylaminopropyl)-N′-ethylcarbodiimide hydrochloride (7.8 g, 40.93 mmol). The reaction mixture was stirred for 3 h, washed with water (100 mL×2), dried over anhydrous sodium sulfate, filtered and concentrated to give 12.0 g of a white solid (compound 14, 100% yield).

### Example 9. Synthesis of di-tert-butyl (((S)-5-((6-aminohexyl)amino)-1-(tert-butoxy)-1, 5-dioxopentan-2-yl) carbamoyl)-L- glutamate (16)

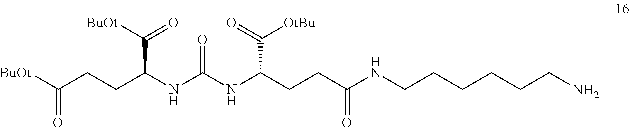

To a stirred solution of 14 (12.0 g, 103.26 mmol) in DCM (150 mL) at 0° C. was added a mixture of hexane-1, 6-diamin.e (12.0 g, 20.49 mmol) in DCM (100 mL) dropwise over 1 h, and the reaction mixture was stirred at room temperature for 2 h, washed with water (100 mLx 2), brine (100 mL), dried over anhydrous sodium sulfate, filtered and concentrated to give the crude, which was purified by a silica gel column, eluted with DCM and methanol, concentrated to afford 8.2 g of a light yellow liquid (compound 16, 100% yield).

### Example 10. Synthesis of tri-tert-butyl (13S, 17S)-1-(4-(((4S, 7S)-7-(((benzyloxy) carbonyl)amino)-4-isopropyl-3, 6, 13-trioxo-1-phenyl-2, 16, 19-trioxa-5, 12-diazahenicosan-21-yl) carbamoyl)phenyl)-1, 10, 15-trioxo-2, 9, 14, 16-tetraazanonadecane-13, 17, 19-trⁱcarboxylate (17)

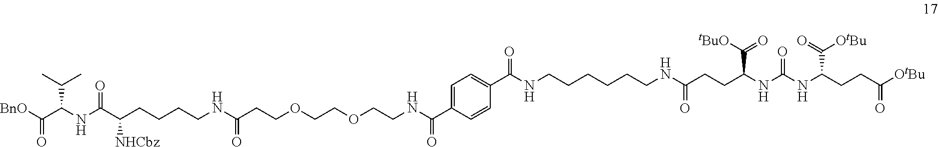 [SMILES: CC(C)[C@H](NC(=O)[C@H](CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)NC(=O)OCc1ccccc1)C(=O)OCc1ccccc1]

To a stirred solution of 16 (725 mg, 1.236 mmol) and 9 (960 mg, 1.236 mmol) in DMF (40 mL) were added HATU (705 mg, 1.853 mmol) and DIEA (480 mg, 3.707 mmol). The mixture was stirred at room temperature for 1 h, diluted with ethyl acetate (100 mL), washed with water and brine, dried over anhydrous sodium sulfate, and evaporated under reduced pressure. The residue was purified by flash column chromatography (silica gel, 0-10% MeOH in DCM) to afford 1.12 g of compound 17 (67% yield) as a yellow oil.

### Example 11. Synthesis of ((S)-17-amino-1-(4-(((7S, 11S)-7, 11-bis(tert-butoxycarbonyl)-2, 2-dimethyl-4, 9, 14-trioxo-3-oxa-8, 10, 15-triazahenicosan-21-yl) carbamoyl)phenyl)-1, 11-dioxo-5, 8-dioxa-2, 12-diazaoctadecan-18-oyl)-L-valine (18)

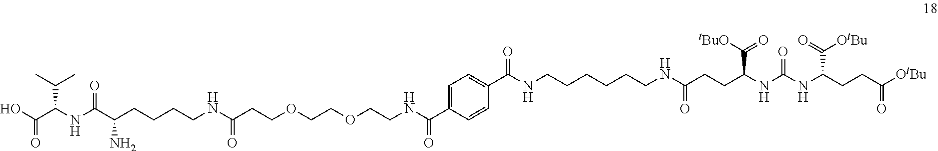 [SMILES: CC(C)[C@H](NC(=O)[C@@H](N)CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)C(=O)O]

To a solution of 17 (1.12 g, 0.832 mmol) in PrOH (100 mL) was added Pd/C (10 wt %, 0.22 g) at r.t. The mixture was stirred at room temperature for 16 h under hydrogen, filtered through Celite, washed with ⁱPrOH, and evaporated under reduced pressure to afford 0.80 g of a white solid (compound 18, 86% yield).

### Example 12. Synthesis of ((S)-1-(4-(((7S, 11S)-7, 11-bis(tert-butoxycarbonyl)-2, 2-dimethyl-4, 9, 14-trioxo-3-oxa-8, 10, 15-triazahenicosan-21-yl) carbamoyl)phenyl)-17-(4-(2, 5-dioxo-2, 5-dihydro-1H-pyrrol-1-yl) butanamido)-1, 11-dioxo-5, 8-dioxa-2, 12-diazaoctadecan-18-oyl)-L-valine (20)

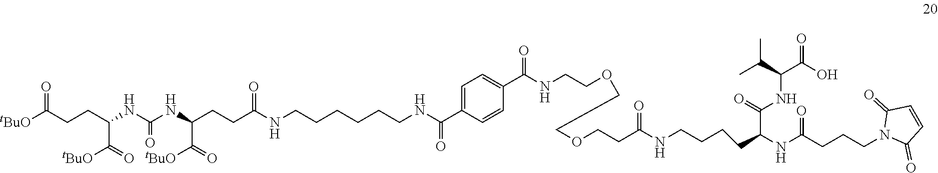 [SMILES: CC(C)[C@H](NC(=O)[C@H](CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)O]

To a stirred solution of 17 (460 mg, 0.410 mmol) and 2, 5-dioxo-2, 5-dihydro-1H-pyrrol-1-yl 4-(2, 5-dioxo-2, 5-dihydro-1H-pyrrol-1-yl) butanoate (138 mg, 0.492 mmol) in DMF (10 mL) was added DIEA (160 mg, 1.231 mmol). The mixture was stirred at room temperature for 2 h, and evaporated under reduced pressure. The residue was purified by flash column chromatography (silica gel, 0-20% MeOH in DCM) to afford 320 mg of a white solid (compound 20, 61% yield).

### Example 13. Synthesis of tri-tert-butyl (13S, 17S)-1-(4-(((15S, 18S, 21S)-22-((5-((R)-5-(tert-butoxy)-2-((tert-butoxycarbonyl)amino)-5-oxopentyl)-2-hydroxyphenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 10, 15-trioxo-2, 9, 14, 16-tetraazanonadecane-13, 17, 19-tricarboxylate (22)

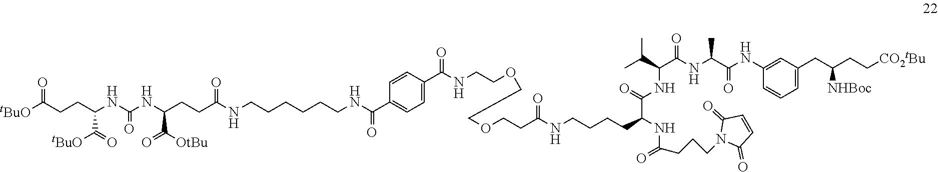 [SMILES: CC(C)[C@H](NC(=O)[C@H](CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)N[C@@H](C)C(=O)Nc1cccc(C[C@@H](CCC(=O)OC(C)(C)C)NC(=O)OC(C)(C)C)c1]

To a stirred solution of 20 (113 mg, 0.088 mmol) and tert-butyl (R)-5-(3-((S)-2-aminopropanamido)-4-hydroxyphenyl)-4-((tert-butoxycarbonyl)amino) pentanoate (40 mg, 0.088 mmol) in DCM (10 mL) were added HATU (37 mg, 0.097 mmol) and DIEA (17 mg, 0.132 mmol). The mixture was stirred at room temperature for 1 h, washed with brine, dried over anhydrous sodium sulfate, and evaporated under reduced pressure. The residue was purified by flash column chromatography (silica gel, 0-30% MeOH in DCM) to afford 120 mg of a yellow oil (compound 22, 79% yield).

### Example 14. Synthesis of (13S, 17S)-1-(4-(((15S, 18S, 21S)-22-((5-((R)-2-amino-4-carboxybutyl)-2-hydroxyphenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 10, 15-trioxo-2, 9, 14, 16-tetraazanonadecane-13, 17, 19-tricarboxylic acid (23)

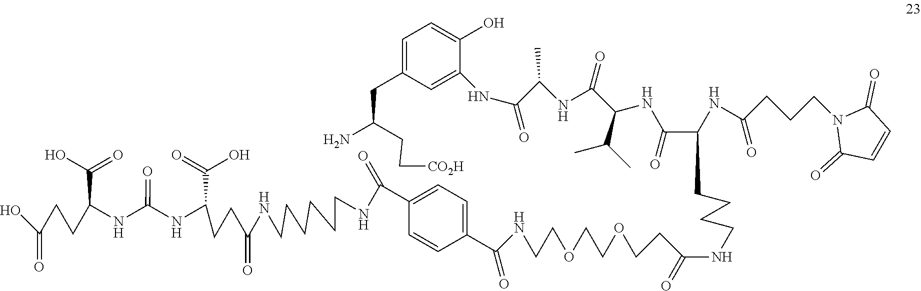 [SMILES: CC(C)[C@H](NC(=O)[C@H](CCCCNC(=O)CCOCCOCCNC(=O)c1ccc(C(=O)NCCCCCCNC(=O)CC[C@H](NC(=O)N[C@@H](CCC(=O)O)C(=O)O)C(=O)O)cc1)NC(=O)CCCN1C(=O)C=CC1=O)C(=O)N[C@@H](C)C(=O)Nc1cc(C[C@H](N)CCC(=O)O)ccc1O]

To a stirred solution of 22 (120 mg, 0.070 mmol) in DCM (1 mL) was added TFA (5 mL). The mixture was stirred at room temperature for 2 h, concentrated in vacuo to afford 97 mg of a yellow oil (23, 100% yield). Example 15 Synthesis of (13S, 17S)-1-(4-(((15S, 18S, 21S)-22-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 10, 15-trioxo-2, 9, 14, 16-tetraazanonadecane-13, 17, 19-tricarboxylic acid (25)

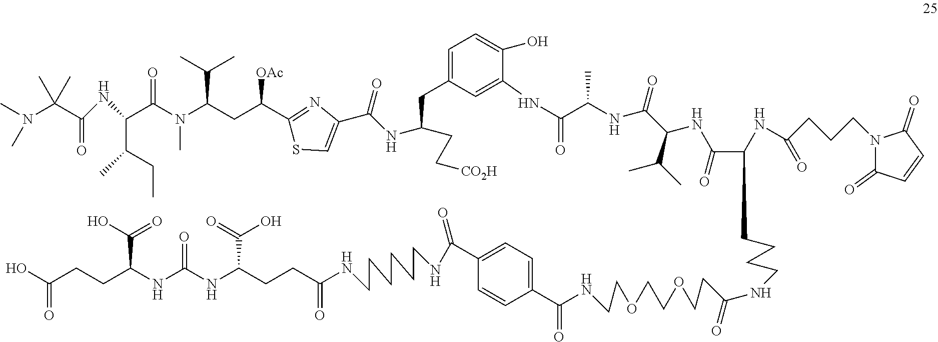 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)[C@H](C)NC(=O)[C@@H](NC(=O)[C@H](CCCCNC(=O)CCOCCOCCNC(=O)c3ccc(C(=O)NCCCCCCNC(=O)CC[C@H](NC(=O)N[C@@H](CCC(=O)O)C(=O)O)C(=O)O)cc3)NC(=O)CCCN3C(=O)C=CC3=O)C(C)C)c2)cs1)C(C)C]

To a stirred solution of 23 (97 mg, 0.070 mmol) and perfluorophenyl 2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxylate (48 mg, 0.070 mmol) in DMF (3 mL) was added DIEA (90 mg, 0.695 mmol). The mixture was stirred at room temperature for 1 h, and HCOOH (0.1 mL) was added. The residue was purified by prep-HPLC and lyophilized to afford 35 mg of compound 25 (26% yield) as a white solid.

### Example 16 Synthesis of tri-tert-butyl (13S, 17S)-1-(4-(((5S, 8S, 11S)-1-((5-((R)-5-(tert-butoxy)-2-((tert-butoxycarbonyl)amino)-5-oxopentyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 10, 15-trioxo-2, 9, 14, 16-tetraazanonadecane-13, 17, 19-tricarboxylate (28)

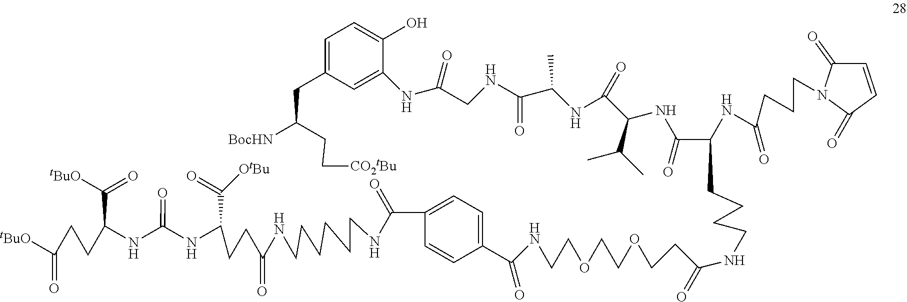 [SMILES: CC(C)[C@H](NC(=O)[C@H](CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)N[C@@H](C)C(=O)NCC(=O)Nc1cc(C[C@@H](CCC(=O)OC(C)(C)C)NC(=O)OC(C)(C)C)ccc1O]

To a stirred solution of 27 (130 mg, 101.0 mmol) and 20 (52.0 mg, 121.3 mmol) in DMF (3 mL) were added HATU (46 mg, 121.3 mmol) and DIEA (39 mg, 303.3 mmol). The mixture was stirred at room temperature for 1 h, and HCOOH (0.1 mL) was added. The reaction mixture was purified by prep-HPLC and lyophilized to afford 100 mg of a white solid (compound 28, 56% yield).

### Example 17 Synthesis of (13S, 17S)-1-(4-(((5S, 8S, 11S)-1-((5-((R)-2-amino-4-carboxybutyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 10, 15-trioxo-2, 9, 14, 16-tetraazanonadecane-13, 17, 19-tricarboxylic acid (29)

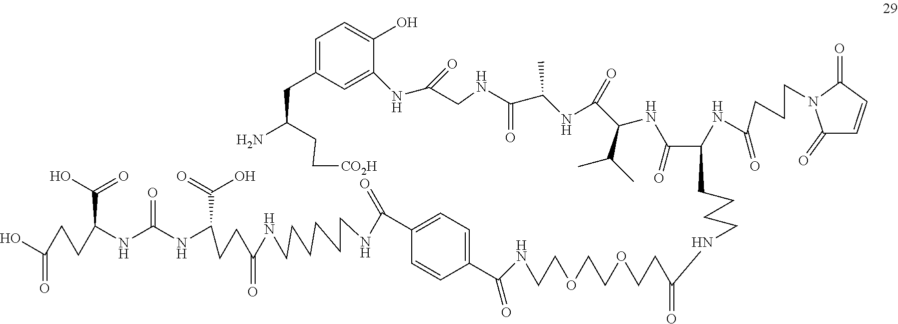 [SMILES: CC(C)[C@H](NC(=O)[C@H](CCCCNC(=O)CCOCCOCCNC(=O)c1ccc(C(=O)NCCCCCCNC(=O)CC[C@H](NC(=O)N[C@@H](CCC(=O)O)C(=O)O)C(=O)O)cc1)NC(=O)CCCN1C(=O)C=CC1=O)C(=O)N[C@@H](C)C(=O)NCC(=O)Nc1cc(C[C@H](N)CCC(=O)O)ccc1O]

To a stirred solution of 28 (100 mg, 56.27 mmol) in DCM (3 mL) was added TFA (3 mL). The mixture was stirred at room temperature for 1 h, concentrated to afford 82 mg of a white solid (compound 29, 100% yield).

### Example 18 Synthesis of(13S, 17S)-1-(4-(((5S, 8S, 11S)-1-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 10, 15-trioxo-2, 9, 14, 16-tetraazanonadecane-13, 17, 19-tricarboxylic acid (30)

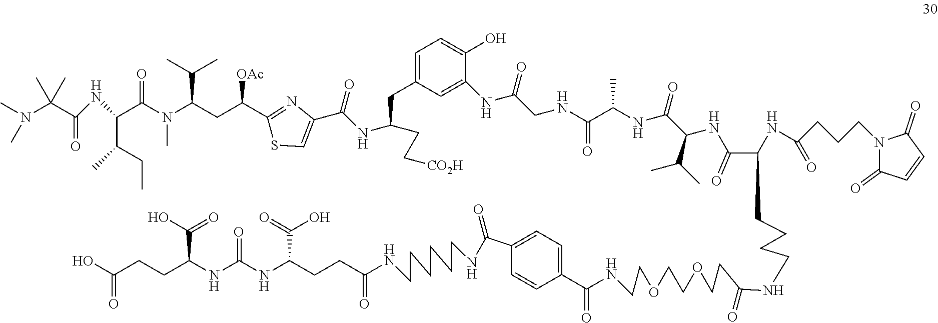 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)CNC(=O)[C@H](C)NC(=O)[C@@H](NC(=O)[C@H](CCCCNC(=O)CCOCCOCCNC(=O)c3ccc(C(=O)NCCCCCCNC(=O)CC[C@H](NC(=O)N[C@@H](CCC(=O)O)C(=O)O)C(=O)O)cc3)NC(=O)CCCN3C(=O)C=CC3=O)C(C)C)c2)cs1)C(C)C]

To a stirred solution of 29 (81.7 mg, 56.2 mmol) in DMF (3 mL) were added 24 (43 mg, 61.9 mmol), and DIEA (73 mg, 562.4 mmol). The mixture was stirred at room temperature for 1 h, and HCOOH (0.1 mL) was added, and then purified by prep-HPLC and lyophilized to afford 50 mg of a white solid (compound 30, 45% yield).

### Example 19 Synthesis of tri-tert-butyl (9S, 13S)-3, 11-dioxo-1-phenyl-2-oxa-4, 10, 12-triazapentadecane-9, 13, 15-tricarboxylate (33)

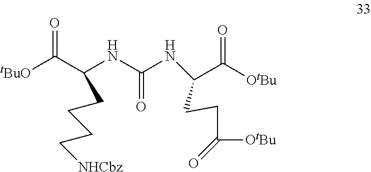 [SMILES: CC(C)(C)OC(=O)CC[C@H](NC(=O)N[C@@H](CCCCNC(=O)OCc1ccccc1)C(=O)OC(C)(C)C)C(=O)OC(C)(C)C]

Under N₂, to a solution of triphosgene (6.86 g, 23.1 mmol, 0.34 eq) in DCM (70 mL) was added dropwise a solution of di-tert-butyl L- glutamate (20.00 g, 67.6 mmol, 1.0 eq) in DCM (70 mL) at −40° C. Then DIEA (35.2 g, 270.5 mmol, 4.0 eq) was added dropwise at −40° C. and stirred for 1 h. A solution of tert-butyl-((benzyloxy) carbonyl)-L-lysinate (25.2 g, 67.6 mmol, 1.0 eq) in DCM (160 mL) was added dropwise to the reaction mixture at −40° C. The mixture was naturally warmed to room temperature and stirred overnight, and washed with water (200 mL). The combined organic layers were dried over anhydrous sodium sulfate and concentrated in vacuo. The residue was purified by column chromatography on silica gel (eluted with petroleum ether: ethyl acetate=4:1) to afford 37.62 g of a yellow oil (compound 33, 89% yield). ESI-MS m/z: calcd. for C₃₂H₅₁N₃O₉[M+H]⁺ 622.4; found 622.2.

### Example 20 Synthesis of tri-tert-butyl (9S, 13S)-3, 11-dioxo-1-phenyl-2-oxa-4, 10, 12-triazapentadecane-9, 13, 15-tricarboxylate (34)

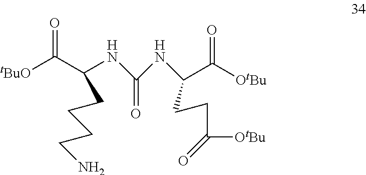 [SMILES: CC(C)(C)OC(=O)CC[C@H](NC(=O)N[C@@H](CCCCN)C(=O)OC(C)(C)C)C(=O)OC(C)(C)C]

To a solution of 33 (15.2 g, 24 mmol, 1.0 eq) in MeOH (150 mL) was added Pd/C (1.5 g, 10 wt %).

The mixture was stirred at room temperature under H₂ for 2 h and filtered over celite. The filtrate was concentrated in vacuo to afford 13.2 g of compound 34. ESI-MS m/z: calcd. for C₂₄H₄₅N₃O₇ [M+H]⁺ 488.3; found 488.1.

### Example 21 Synthesis of tri-tert-butyl (15S, 19S)-3, 9, 17-trioxo-1-phenyl-2-oxa-4, 10, 16, 18-tetraazahenicosane-15, 19, 21-tricarboxylate (36)

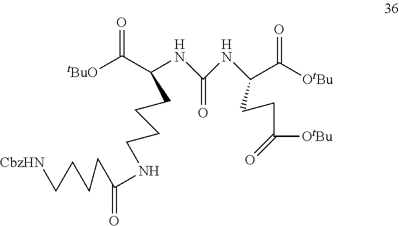 [SMILES: CC(C)(C)OC(=O)CC[C@H](NC(=O)N[C@@H](CCCCNC(=O)CCCCNC(=O)OCc1ccccc1)C(=O)OC(C)(C)C)C(=O)OC(C)(C)C]

To a solution of 34 (3.8 g, 7.79 mmol, 1.03 eq) and 35 (1.9 g, 7.56 mmol, 1.0 eq) in DCM (50 mL) were added HATU (4.31 g, 11.34 mmol, 1.5 eq) and DIEA (1.95 g, 15.12 mmol, 2.0 eq). The mixture was stirred at room temperature for 2 h and quenched with water (50 mL) and extracted with DCM (50 mL×3). The combined organic layers were washed with brine (50 mL×3), dried over anhydrous sodium sulfate and concentrated in vacuo. The residue was purified by column chromatography, eluted with petroleum ether: ethyl acetate (3:1) to afford 2.6 g of a colorless oil (compound 36, 48% yield). ESI-MS m/z: calcd. for C₃₇H₆₀N₄O₁₀ [M+H]⁺ 721.4; found 721.1.

### Example 22 Synthesis of di-tert-butyl (((S)-6-(5-aminopentanamido)-1-(tert-butoxy)-1-oxohexan-2-yl) carbamoyl)-L- glutamate (37)

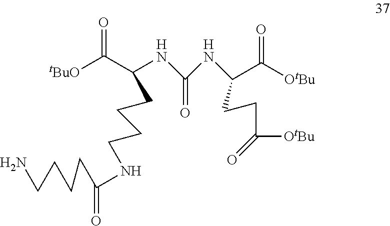 [SMILES: CC(C)(C)OC(=O)CC[C@H](NC(=O)N[C@@H](CCCCNC(=O)CCCCN)C(=O)OC(C)(C)C)C(=O)OC(C)(C)C]

To a solution of 36 (2.6 g, 3.6 mmol, 1.0 eq) in MeOH (60 mL) was added Pd/C (260 mg, 10 wt %). The mixture was stirred at room temperature under H₂ for 2 h and filtered over celite. The filtrate was concentrated in vacuo to afford 2.04 g of a brown oil (compound 37, 96% yield). ESI-MS m/z: calcd. for C₂₉H₅₄N4O₈ [M+H]⁺ 587.4; found 587.1.

### Example 23 Synthesis of tri-tert-butyl (13S, 17S)-1-(4-(((4S, 7S)-7-(((benzyloxy) carbonyl)amino)-4-isopropyl-3, 6, 13-trioxo-1-phenyl-2, 16, 19-trioxa-5, 12-diazahenicosan-21-yl) carbamoyl)phenyl)-1, 7, 15-trioxo-2, 8, 14, 16-tetraazanonadecane-13, 17, 19-tricarboxylate (38)

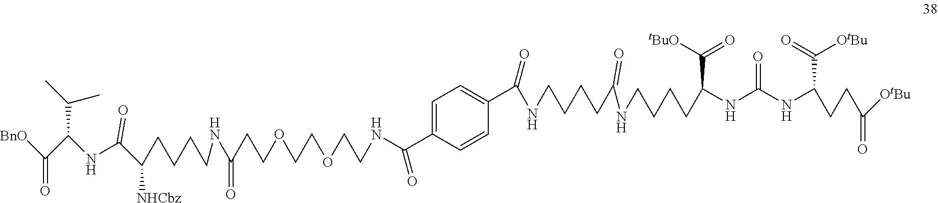 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)NC(=O)OCc1ccccc1)C(=O)OCc1ccccc1]

To a solution of 37 (763 mg, 1.30 mmol, 1.0 eq) and 9 (1.01 g, 1.30 mmol, 1.0 eq) in DCM (10 mL) were added HATU (593 mg, 1.56 mmol, 1.2 eq) and DIEA (504 mg, 3.90 mmol, 3.0 eq). The mixture was stirred at room temperature for 2 h and quenched with water (50 mL) and extracted with DCM (30 mL×3). The combined organic layers were washed with brine (30 mL×3), dried over anhydrous sodium sulfate and concentrated in vacuo. The residue was purified by column chromatography (DCM: MeOH=15:1) to afford 1.01 g of a white solid (compound 37, 58% yield). ESI-MS m/z: calcd. for C₇₀H₁₀₄N₈O₁₈ [M+H]⁺ 1345.8; found 1346.3.

### Example 24 Synthesis of((S)-17-amino-1-(4-(((7S, 11S)-7, 11-bis(tert-butoxycarbonyl)-2, 2-dimethyl-4, 9, 17-trioxo-3-oxa-8, 10, 16-triazahenicosan-21-yl) carbamoyl)phenyl)-1, 11-dioxo-5, 8-dioxa-2, 12-diazaoctadecan-18-oyl)-L-valine (39)

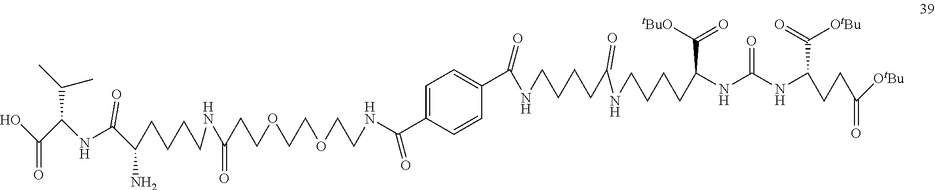 [SMILES: CC(C)[C@H](NC(=O)[C@@H](N)CCCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)C(=O)O]

To a solution of 37 (1.01 g, 0.75 mmol, 1.0 eq) in isopropyl alcohol (50 mL) was added Pd/C (100 mg, 10 wt %). The mixture was stirred at 50° C. under H₂ for 5 h and filtered over celite. The filtrate was concentrated in vacuo to afford 860 mg of a brown solid (compound 39, 102% yield). ESI-MS m/z: calcd. for C₅₅H₉₂N₈O₁₆ [M+H]⁺ 1121.7; found 1122.0.

### Example 25 synthesis of ((S)-1-(4-(((7S, 11S)-7, 11-bis(tert-butoxycarbonyl)-2, 2-dimethyl-4, 9, 17-trioxo-3-oxa-8, 10, 16-triazahenicosan-21-yl) carbamoyl)phenyl)-17-(4-(2, 5-dioxo-2, 5-dihydro-1H-pyrrol-1-yl) butanamido)-1, 11-dioxo-5, 8-dioxa-2, 12-diazaoctadecan-18-oyl)-L-valine (40)

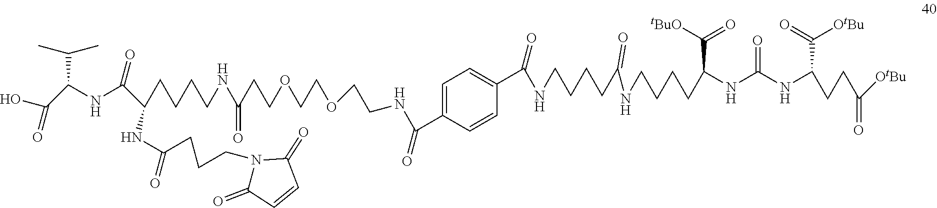 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)O]

To a solution of 39 (600 mg, 0.54 mmol, 1.0 eq) in DMF (5 mL) was added DIEA (207 mg, 1.61 mmol, 3.0 eq), followed by 19 (180 mg, 0.64 mmol, 1.2 eq). The mixture was stirred at room temperature for 1.5 h and then concentrated in vacuo. The residue was purified by column chromatography on silica, eluted with DCM: MeOH=10:1-5:1, to afford 553 mg of a white solid (compound 40, 80% yield). ESI-MS m/z: calcd. for C₆₃H₉₉N₉O₁₉ [M+H]⁺ 1286.7; found 1287.2.

### Example 26 synthesis of tri-tert-butyl (13S, 17S)-1-(4-(((15S, 18S, 21S)-22-((5-((R)-5-(tert-butoxy)-2-((tert-butoxycarbonyl)amino)-5-oxopentyl)-2-hydroxyphenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylate (41)

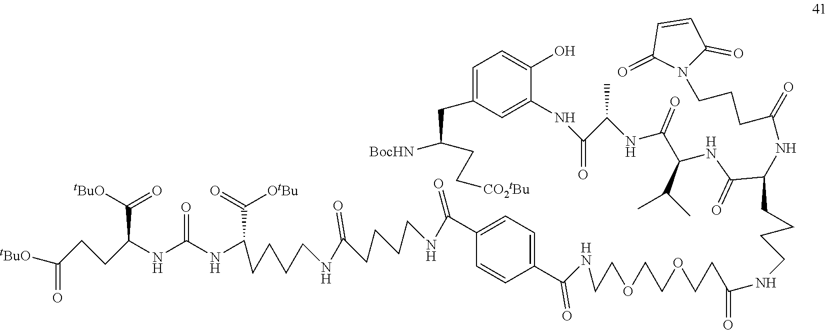 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)N[C@@H](C)C(=O)Nc1cc(C[C@@H](CCC(=O)OC(C)(C)C)NC(=O)OC(C)(C)C)ccc1O]

To a solution of 40 (171 mg, 0.133 mmol, 1.0 eq) and 21 (62 mg, 0.133 mmol, 1.0 eq) in DMF (3 mL) were added HATU (61 mg, 0.159 mmol, 1.2 eq), and DIEA (52 mg, 0.399 mmol, 3.0 eq). The mixture was stirred at room temperature for 1 h, quenched with water (20 mL), extracted with DCM (20 mL×3). The combined organic layers were washed with brine (20 mL×3), dried over anhydrous sodium sulfate and concentrated in vacuo. The residue was purified column chromatography on silica, eluted with DCM: MeOH=10:1-5:1 to afford 52 mg of a white solid (compound 41, 23% yield). ESI-MS m/z: calcd. for C₈₆H₁₃₄N₁₂O₂₄ [M+H]⁺ 1720.0; found 1720.5.

### Example 27 synthesis of (13S, 17S)-1-(4-(((15S, 18S, 21S)-22-((5-((R)-2-amino-4-carboxybutyl)-2-hydroxyphenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylic acid (42)

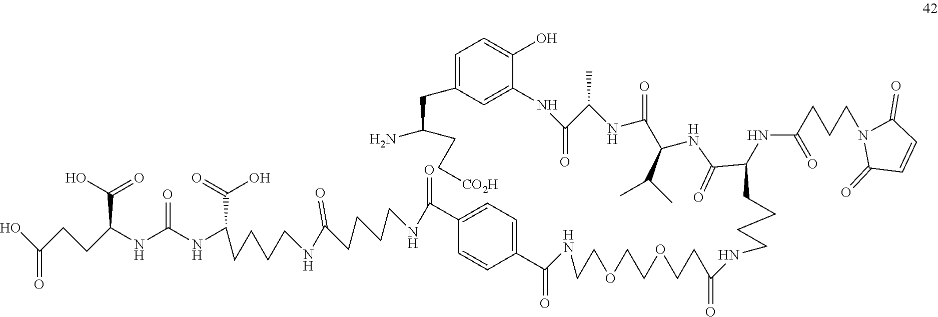 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)O)C(=O)O)C(=O)O)cc1)NC(=O)CCCN1C(=O)C=CC1=O)C(=O)N[C@@H](C)C(=O)Nc1cc(C[C@H](N)CCC(=O)O)ccc1O]

To a solution of 41 (69 mg, 0.04 mmol) in DCM (4 mL) was added TFA (3 mL). The mixture was stirred at room temperature for 1 h and concentrated in vacuo to afford 84 mg of a white solid (compound 42, crude), which was used for the next step without further purification. ESI-MS m/z: calcd. for C₆₅H₉₄N₁₂O₂₂ [M+H]⁺ 1395.7; found 1396.3.

### Example 28 synthesis of (13S, 17S)-1-(4-(((15S, 18S, 21S)-22-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylic acid (43)

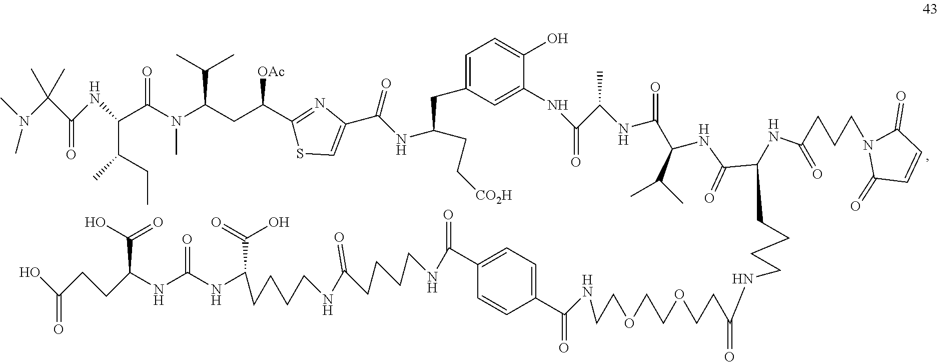 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)[C@H](C)NC(=O)[C@@H](NC(=O)[C@H](CCCCNC(=O)CCOCCOCCNC(=O)c3ccc(C(=O)NCCCCC(=O)NCCCC[C@H](NC(=O)N[C@@H](CCC(=O)O)C(=O)O)C(=O)O)cc3)NC(=O)CCCN3C(=O)C=CC3=O)C(C)C)c2)cs1)C(C)C]

To a solution of 42 (56 mg, 0.04 mmol, 1.0 eq, crude) and 24 (27.8 mg, 0.04 mmol, 1.0 eq) in DMF (2 mL) was added DIEA (25.93 mg, 0.20 mmol, 5.0 eq). The mixture was stirred at room temperature for 1 h, and purified by prep-HPLC and lyophilized to afford 49 mg of compound 43 (65% over two steps) as a white solid. ESI-MS m/z: calcd. for C₉₀H₁₃₄N₁₆O₂₇S [M+H]⁺ 1903.9; found 1904.9.

### Example 29 synthesis of tri-tert-butyl (13S, 17S)-1-(4-(((5S, 8S, 11S)-1-((5-((R)-5-(tert-butoxy)-2-((tert-butoxycarbonyl)amino)-5-oxopentyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 7, 15-trioxo-2, 8, 14, 16-tetraazanonadecane-13, 17, 19-tricarboxylate (45)

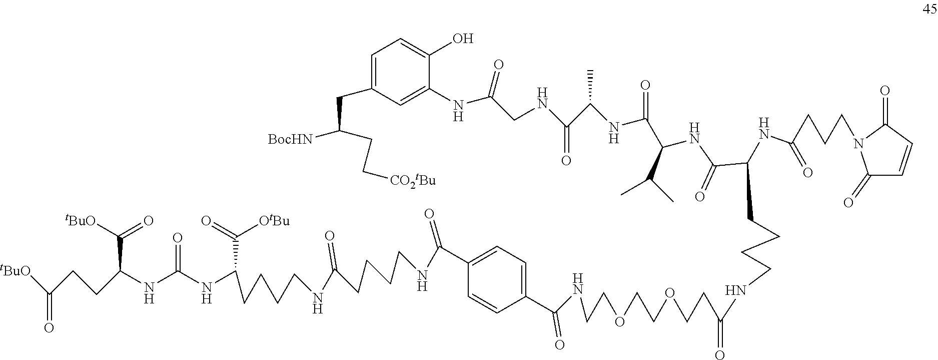 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)N[C@@H](C)C(=O)NCC(=O)Nc1cc(C[C@@H](CCC(=O)OC(C)(C)C)NC(=O)OC(C)(C)C)ccc1O]

To a solution of 27 (100 mg, 0.078 mmol, 1.0 eq) and 40 (40 mg, 0.078 mmol, 1.0 eq) in CH₃CN (3 mL) were added NMI (13 mg, 0.155 mmol, 1.2 eq) and TCFH (26 mg, 0.093 mmol, 2.0 eq). The mixture was stirred at room temperature for 2 h and purified by prep-HPLC and lyoplilized to afford 45 mg of a white solid (compound 45, 32% yield). ESI-MS m/z: calcd. for C₈₈H₁₃₇N₁₃O₂₅ [M+H]⁺ 1777.0; found 1777.1.

### Example 30 synthesis of (13S, 17S)-1-(4-(((5S, 8S, 11S)-1-((5-((R)-2-amino-4-carboxybutyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 7, 15-trioxo-2, 8, 14, 16-tetraazanonadecane-13, 17, 19-tricarboxylic acid (46)

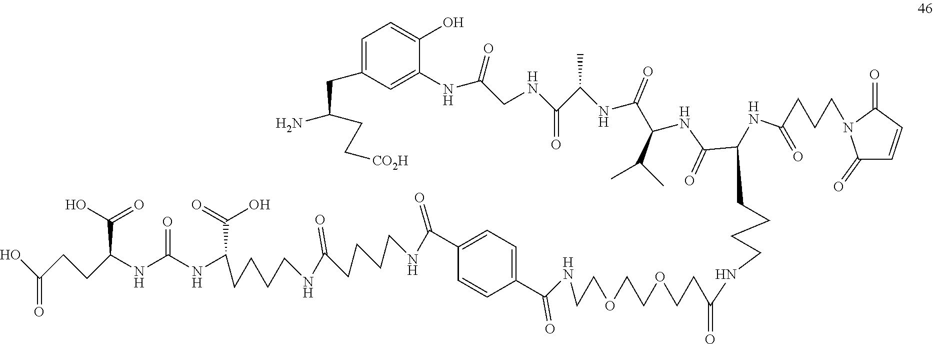 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)O)C(=O)O)C(=O)O)cc1)NC(=O)CCCN1C(=O)C=CC1=O)C(=O)N[C@@H](C)C(=O)NCC(=O)Nc1cc(C[C@H](N)CCC(=O)O)ccc1O]

To a solution of 45 (46 mg, 0.026 mmol) in DCM (3 mL) was added TFA (2 mL). The mixture was stirred at room temperature for 0.5 h and then concentrated in vacuo to afford 43 mg of a white solid (compound 46, crude), which was used for the next step without further purification. ESI-MS m/z: calcd. for C₆₇H₉₇N₁₃O₂₃ [M+H]⁺ 1452.7; found 1452.9.

### Example 31 synthesis of (13S, 17S)-1-(4-(((5S, 8S, 11S)-1-((5-((R)-2-(2-((6S, 9R, I 1R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 7, 15-trioxo-2, 8, 14, 16-tetraazanonadecane-13, 17, 19-tricarboxylic acid (47)

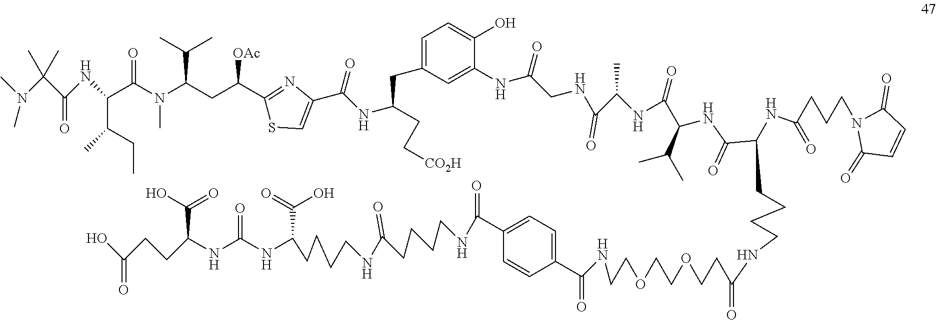 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)CNC(=O)[C@H](C)NC(=O)[C@@H](NC(=O)[C@H](CCCCNC(=O)CCOCCOCCNC(=O)c3ccc(C(=O)NCCCCC(=O)NCCCC[C@H](NC(=O)N[C@@H](CCC(=O)O)C(=O)O)C(=O)O)cc3)NC(=O)CCCN3C(=O)C=CC3=O)C(C)C)c2)cs1)C(C)C]

To a solution of 46 (37 mg, 0.025 mmol, 1.0 eq, crude) and 24 (18 mg, 0.025 mmol, 1.0 eq) in DMF (1 mL) was added DIEA (16 mg, 0.127 mmol, 5.0 eq). The mixture was stirred at room temperature for I h and purified by prep-HPLC and lyophilized to afford 25 mg of a white solid (compound 47, 25% over two steps). ESI-MS m/z: calcd. C₉₂H₁₃₇N₁₇O₂₈S [M+H]⁺ 1961.0; found 1963.1.

### Example 32. Synthesis of ((S)-1-(4-(((7S, 1IS)-7, 11-bis(tert-butoxycarbonyl)-2, 2-dimethyl-4, 9, 17-trioxo-3-oxa-8, 10, 16-triazahenicosan-21-yl) carbamoyl)phenyl)-17-(2-(2, 5-dioxo-2, 5-dihydro-1H-pyrrol-1-yl) acetamido)-1, 11-dioxo-5, 8-dioxa-2, 12-diazaoctadecan-18-oyl)-L-valine (49)

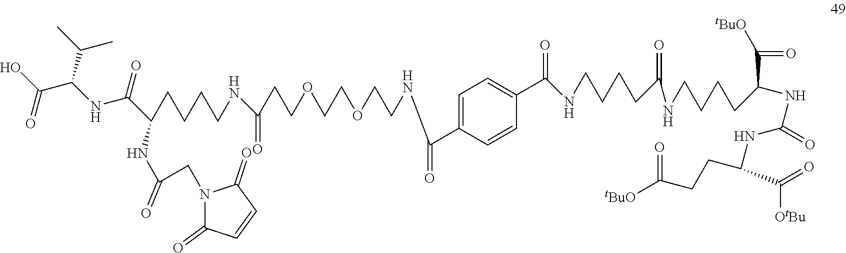 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)NC(=O)CN1C(=O)C=CC1=O)C(=O)O]

To a stirred solution of 39 (1.6 g, 1.42 mmol) in DMF (30 mL) were added 19 (0.72 g, 2.85 mmol) and DIEA (0.37 g, 2.85 mmol) at room temperature. The reaction mixture was stirred at room temperature for I h, purified by prep-HPLC, lyophilized to afford 49 (1.1 g, 64% yield) as a white solid.

### Example 33. Synthesis of tri-tert-butyl (13S, 17S)-1-(4-(((15S, 18S, 21S)-22-((5-((R)-5-(tert-butoxy)-2-((tert-butoxycarbonyl)amino)-5-oxopentyl)-2-hydroxyphenyl)amino)-15-(2-(2, 5-dioxo-2, 5-dihydro-IH-pyrrol-1-yl) acetamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylate (50)

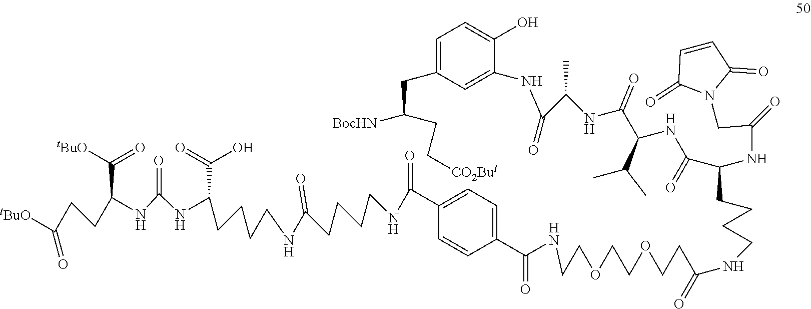

To a stirred solution of 49 (570 mg, 0.45 mmol) in DMF (5 mL) at room temperature were added 21 (205.0 mg, 0.45 mmol) and DIEA (88.0 mg, 0.68 mmol). The reaction mixture was stirred at room temperature for 1 h, purified by prep-HPLC, lyophilized to afford 50 (368 mg, 48% yield) as a pink liquid.

### Example 34. Synthesis of (13S, 17S)-1-(4-(((15S, 18S, 21S)-22-((5-((R)-2-amino-4-carboxybutyl)-2-hydroxyphenyl)amino)-15-(2-(2, 5-dioxo-2, 5-dihydro-1H-pyrrol-1-yl) acetamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylic acid (51)

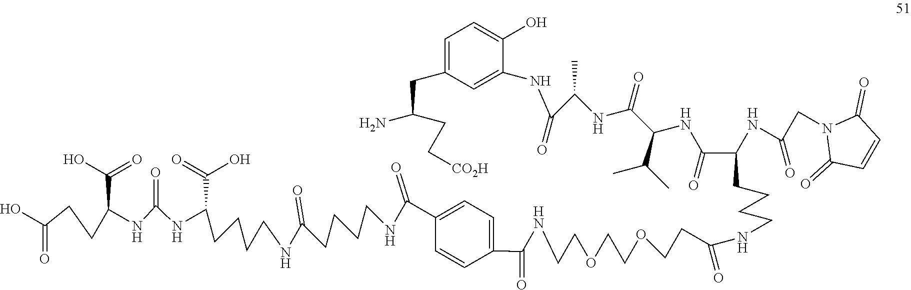 [SMILES: CC(C)[C@H](NC(=O)[C@H](CCCCNC(=O)CCOCCOCCNC(=O)c1ccc(C(=O)NCCCCC(=O)NCCCC[C@H](NC(=O)N[C@@H](CCC(=O)O)C(=O)O)C(=O)O)cc1)NC(=O)CN1C(=O)C=CC1=O)C(=O)N[C@@H](C)C(=O)Nc1cc(C[C@H](N)CCC(=O)O)ccc1O]

To a stirred solution of 50 (108.0 mg, 0.064 mmol) in DCM (1 mL) at room temperature was added TFA (5 mL), and the reaction mixture was stirred at room temperature for 2 h, concentrated to afford 51 (87.0 mg, 100% yield) as a colorless oil.

### Example 35. Synthesis of (13S, 17S)-1-(4-(((15S, 18S, 21S)-22-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-15-(2-(2, 5-dioxo-2, 5-dihydro-1H-pyrrol-1-yl) acetamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylic acid (52)

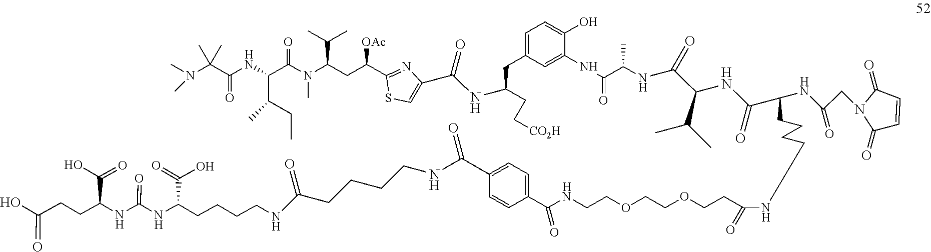 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)[C@H](C)NC(=O)[C@@H](NC(=O)[C@@H](CCCCNC(=O)CCOCCOCCNC(=O)c3ccc(C(=O)NCCCCC(=O)NCCCC[C@H](NC(=O)N[C@@H](CCC(=O)O)C(=O)O)C(=O)O)cc3)NC(=O)CN3C(=O)C=CC3=O)C(C)C)c2)cs1)C(C)C]

To a stirred solution of 51 (87.0 mg, 0.064 mmol) in DMF (10 mL) at 0° C. were added 24 (53.0 mg, 0.076 mmol) and DIEA (100.0 mg, 0.76 mmol). The reaction mixture was stirred at 0° C. for 0.5 h and room temperature for 1 h, purified by prep-HPLC, lyophilized to afford 52 (72.0 mg, 60% yield) as a white solid.

### Example 36. Synthesis of tert-butyl 4-((2-(2-(3-((2, 5-dioxopyrrolidin-1-yl)oxy)-3-oxopropoxy) ethoxy)ethyl) carbamoyl)benzoate (64)

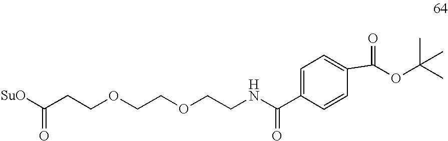

To a stirred solution of 7 (6.4 g, 16.77 mmol) in DCM (100 mL) at room temperature were added EDCI·HCl (6.4 g, 33.53 mmol) and NHS (2.9 g, 25.16 mmol), and the reaction mixture was stirred at room temperature for 1 h, washed with water (100 mL), brine (100 mL), concentrated in vacuo. The residue was purified by a silica gel column, eluted with ethyl acetate and petroleum ether, concentrated to afford 64 (5.7 g, 71% yield) as a colorless oil.

### Example 37. Synthesis of benzyl (19S, 22S, 25S)-19-(((benzyloxy) carbonyl)amino)-1-(4-(tert-butoxycarbonyl)phenyl)-22-isopropyl-25-methyl-1, 11, 16, 20, 23-pentaoxo-5, 8-dioxa-2, 12, 1512, 21, 24-pentaazahexacosan-26-oate (65)

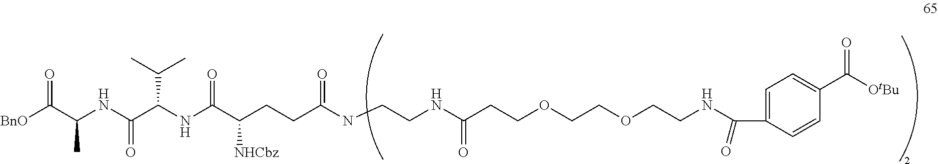

To a stirred solution of 63 (1.25 g, 1.99 mmol) in DMF (20 mL) at room temperature were added DIEA (1.5 g, 11.96 mmol) and 64 (2.60 g, 11.96 mmol). The reaction mixture was stirred at room temperature for 3 h, purified by prep-HPLC, lyophilized to afford 65 (1.2 g, 46% yield) as a pink liquid.

### Example 38. Synthesis of 4-(((4S, 7S, 1OS)-10-(((benzyloxy) carbonyl)amino)-7-isopropyl-4-methyl-3, 6, 9, 13, 18-pentaoxo-1-phenyl-2, 21, 24-trioxa-5, 8, 1412, 17-tetraazahexacosan-26-yl) carbamoyl)benzoic acid (66)

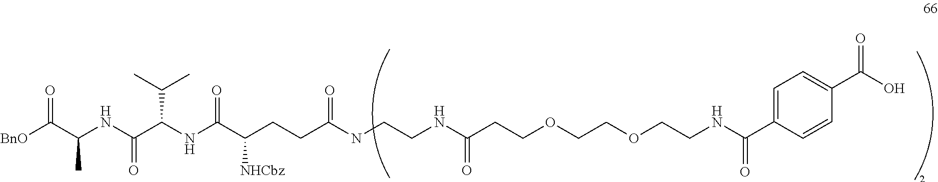

To a stirred solution of 65 (1.24 g, 0.91 mmol) in DCM (5 mL) at room temperature was added formic acid (15 mL). The reaction mixture was stirred at 45° C. overnight, concentrated, purified by a silica gel column, eluted with DCM and methanol, to afford 66 (787 mg, 69% yield) as a white solid.

### Example 39. Synthesis of tri-tert-butyl (13S, 17S)-1-(4-(((4S, 7S, 10S)-10-(((benzyloxy) carbonyl)amino)-7-isopropyl-4-methyl-3, 6, 9, 13, 18-pentaoxo-1-phenyl-2, 21, 24-trioxa-5, 8, 1412, 17-tetraazahexacosan-26-yl) carbamoyl)phenyl)-1, 10, 15-trioxo-2, 9, 14, 16-tetraazanonadecane-13, 17, 19-tricarboxylate (67)

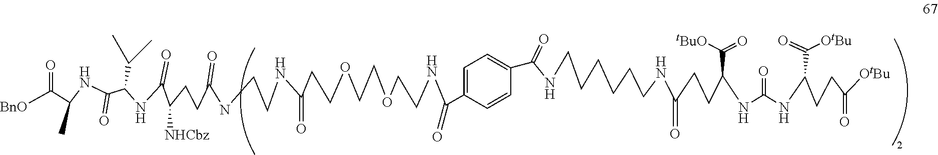

To a stirred solution of 66 (757.0 mg, 0.61 mmol) in DMF (10 mL) at room temperature were added 16 (895.0 mg, 1.52 mmol), HATU (697.0 mg, 1.83 mmol) and DIEA (355.0 mg, 2.74 mmol). The reaction mixture was stirred at room temperature for 0.5 h, diluted with DCM (150 mL), washed with water (100 mL) and concentrated. The residue was purified by a silica gel column, eluted with DCM and methanol, to afford 67 (1.20 g, 82% yield) as a white solid.

### Example 40. Synthesis of ((S)-19-amino-1-(4-(((7S, 11S)-7, 11-bis(tert-butoxycarbonyl)-2, 2-dimethyl-4, 9, 14-trioxo-3-oxa-8, 10, 15-triazahenicosan-21-yl) carbamoyl)phenyl)-1, 11, 16-trioxo-5, 8-dioxa-2, 12, 1512-triazaicosan-20-oyl)-L-valyl-L-alanine (68)

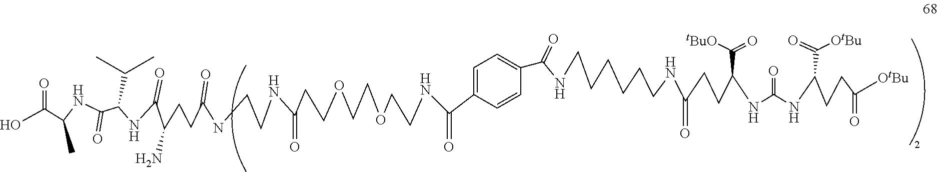

To a stirred solution of 67 (908.0 mg, 0.38 mmol) in isopropyl alcohol (10 mL) was added Pd/C (1.0 g, 10 wt %). The mixture was stirred overnight under a hydrogen balloon at room temperature, filtered over celite. The filtrate was concentrated to give 68 (692 mg, 84% yield) as a brown solid.

### Example 41. Synthesis of ((S)-1-(4-(((7S, 11S)-7, 11-bis(tert-butoxycarbonyl)-2, 2-dimethyl-4, 9, 14-trioxo-3-oxa-8, 10, 15-triazahenicosan-21-yl) carbamoyl)phenyl)-19-((4-(2, 5-dioxo-2, 5-dihydro-1H-pyrrol-1-yl)butyl)amino)-1, 11, 16-trioxo-5, 8-dioxa-2, 12, 1512-triazaicosan-20-oyl)-L-valyl-L-alanine (69)

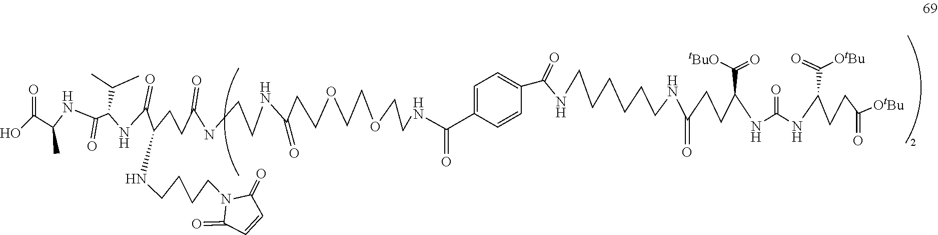

To a stirred solution of 68 (692 mg, 0.321 mmol) in DMF (10 mL) at room temperature were added 19 (135.0 mg, 0.48 mmol) and DIEA (84 mg, 0.64 mmol). The mixture was stirred at room temperature for 2 h and then purified by prep-HPLC, lyophilized to afford 69 (290 mg, 38% yield) as a pink solid.

### Example 42. Synthesis of tri-tert-butyl (13S, 17S)-1-(4-(((2S, 5S, 8S, 11S)-1-((5-((R)-5-(tert-butoxy)-2-((tert-butoxycarbonyl)amino)-5-oxopentyl)-2-hydroxyphenyl)amino)-11-(4-(2, 5-dioxo-2, 5-dihydro-1H-pyrrol-1-yl) butanamido)-8-isopropyl-2, 5-dimethyl-1, 4, 7, 10, 14, 19-hexaoxo-22, 25-dioxa-3, 6, 9, 1512, 18-pentaazaheptacosan-27-yl) carbamoyl)phenyl)-1, 10, 15-trioxo-2, 9, 14, 16-tetraazanonadecane-13, 17, 19-tricarboxylate (70)

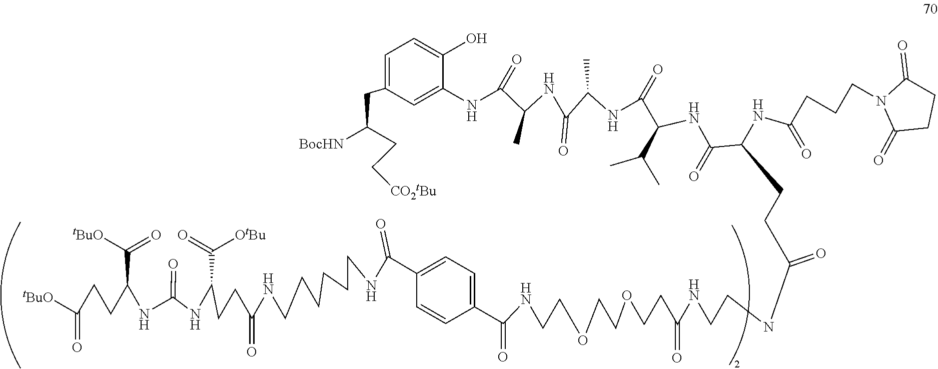

To a stirred solution of 69 (104.0 mg, 0.045 mmol) in DCM (10 mL) at room temperature were added 21 (20.0 mg, 0.045 mmol), HATU (19.0 mg, 0.049 mmol) and DIEA (9 mg, 0.067 mmol). The reaction mixture was stirred at room temperature for 1 h, diluted with DCM (50 mL), washed with water (50 mL), brine (50 mL), concentrated in vacuo. The residue was purified by a silica gel column, eluted with DCM and methanol, to afford 70 (96 mg, 77% yield) as a colorless oil.

### Example 43. Synthesis of (13S, 17S)-1-(4-(((2S, 5S, 8S, 11S)-1-((5-((R)-2-amino-4-carboxybutyl)-2-hydroxyphenyl)amino)-11-(4-(2, 5-dioxo-2, 5-dihydro-1H-pyrrol-1-yl) butanamido)-8-isopropyl-2, 5-dimethyl-1, 4, 7, 10, 14, 19-hexaoxo-22, 25-dioxa-3, 6, 9, 1512, 18-pentaazaheptacosan-27-yl) carbamoyl)phenyl)-1, 10, 15-trioxo-2, 9, 14, 16-tetraazanonadecane-13, 17, 19-tricarboxylic acid (71)

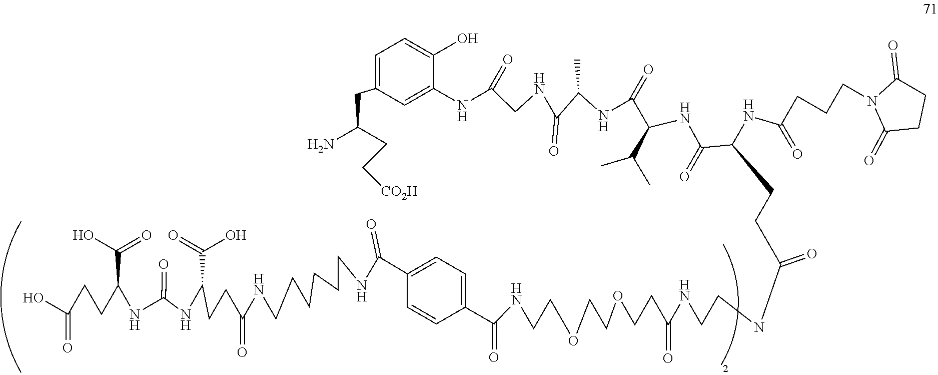

To a stirred solution of 70 (96.0 mg, 0.035 mmol) in DCM (1 mL) at room temperature was added TFA (6 mL). The mixture was stirred at room temperature for 2 h and concentrated in vacuo to afford 71 (79.0 mg, 100% yield) as a colorless oil.

### Example 44. Synthesis of (13S, 17S)-1-(4-(((2S, 5S, 8S, 11S)-1-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-11-(4-(2, 5-dioxo-2, 5-dihydro-1H-pyrrol-1-yl) butanamido)-8-isopropyl-2, 5-dimethyl-1, 4, 7, 10, 14, 19-hexaoxo-22, 25-dioxa-3, 6, 9, 1512, 18-pentaazaheptacosan-27-yl) carbamoyl)phenyl)-1, 10, 15-trioxo-2, 9, 14, 16-tetraazanonadecane-13, 17, 19-tricarboxylic acid (72)

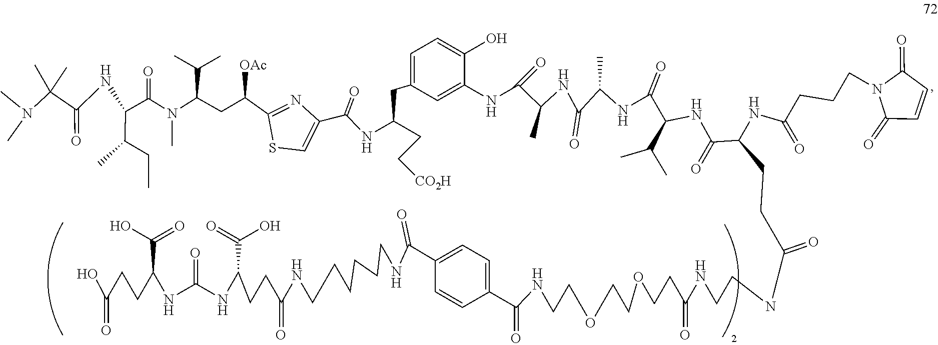

To a stirred solution of 71 (79.0 mg, 0.035 mmol) in DCM (1 mL) at room temperature were added 24 (25.0 mg, 0.035 mmol) and DIEA (23.0 mg, 0.17 mmol). The mixture was stirred at room temperature for 2 h, and concentrated in vacuo to afford 72 (15 mg, 15% yield) as a white solid.

### Example 45. Synthesis of tri-tert-butyl (13S, 17S)-1-(4-(((5S, 8S, 11S)-1-((5-((R)-5-(tert-butoxy)-2-((tert-butoxycarbonyl)amino)-5-oxopentyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 14, 19-hexaoxo-22, 25-dioxa-3, 6, 9, 1512, 18-pentaazaheptacosan-27-yl) carbamoyl)phenyl)-1, 10, 15-trioxo-2, 9, 14, 16-tetraazanonadecane-13, 17, 19-tricarboxylate (75)

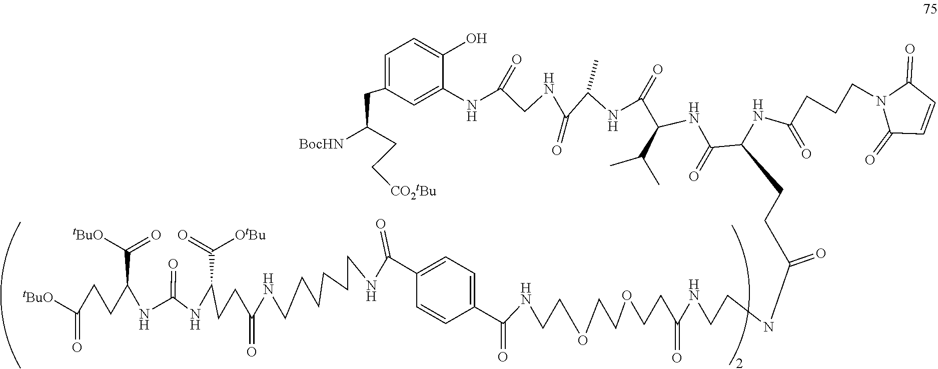

To a solution of compound 69 (210.0 mg, 0.0934 mmol, 1.0 eq) in DMF (3 mL) was added HATU (37.3 mg, 0.0980 mmol, 1.05 eq). The mixture was stirred at room temperature for 10 min. Then compound 74 (47.5 mg, 0.0934 mmol, 1.0 eq) and DIEA (18.1 mg, 0.140 mmol, 1.5 eq) were added. The reaction mixture was stirred at room temperature for 0.5 h and then quenched by HCOOH (0.5 mL), purified by flash column chromatography to give 75 (90.0 mg, 0.0329 mmol, 35% yield) as a white solid. ESI-MS m/z: [M+H]⁺ calcd. for C₁₃₅H₂₁₂N₂₀O₃₉ 2737.52; found 1369.82.

### Example 46. Synthesis of (13S, 17S)-1-(4-(((5S, 8S, 11S)-1-((5-((R)-2-amino-4-carboxybutyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 14, 19-hexaoxo-22, 25-dioxa-3, 6, 9, 1512, 18-pentaazaheptacosan-27-yl) carbamoyl)phenyl)-1, 10, 15-trioxo-2, 9, 14, 16-tetraazanonadecane-13, 17, 19-tricarboxylic acid (76)

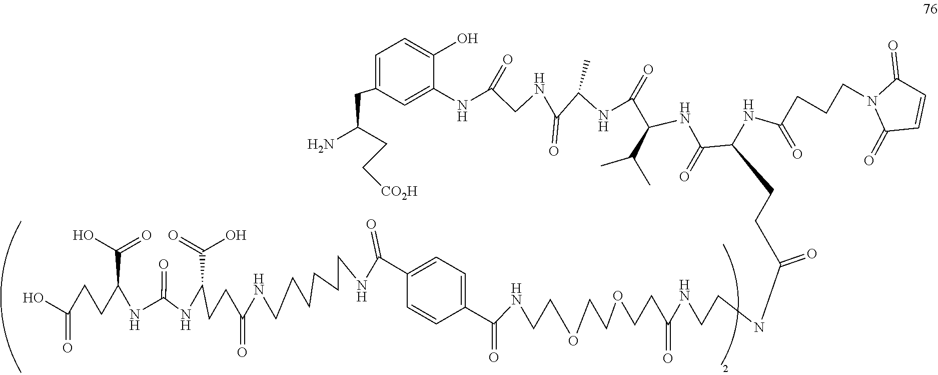

To a solution of compound 75 (90.0 mg, 0.0329 mmol) in DCM (2 mL) was added TFA (2 mL). The mixture was stirred at room temperature for 0.5 h, concentrated under reduced pressure. The residue was washed with petroleum ether (5 mL), and concentrated to give 76 (73.8 mg, 0.0329 mmol, 100% yield) as a light-yellow foam. ESI-MS m/z: [M+H]⁺calcd. for C₁₀₂H₁₄₈N₂₀O₃₇ 2245.03; found 1123.60.

### Example 47. Synthesis of (13S, 17S)-1-(4-(((5S, 8S, 11S)-1-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-11-(4-(2, 5-dioxo-2, 5-dihydro-1H-pyrrol-1-yl) butanamido)-8-isopropyl-5-methyl-1, 4, 7, 10, 14, 19-hexaoxo-22, 25-dioxa-3, 6, 9, 1512, 18-pentaazaheptacosan-27-yl) carbamoyl)phenyl)-1, 10, 15-trioxo-2, 9, 14, 16-tetraazanonadecane-13, 17, 19-tricarboxylic acid (77)

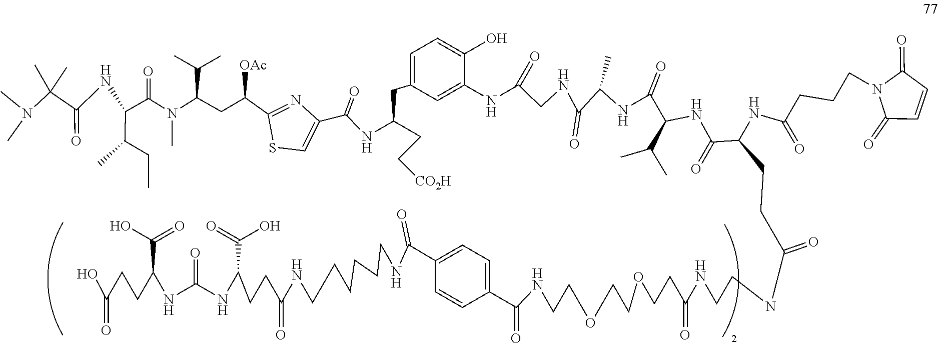

To a solution of compound 76 (74.0 mg, 0.0329 mmol, 1.0 eq) and compound 24 (27.4 mg, 0.0395 mmol, 1.2 mmol) in DMF (3 mL) was added DIEA (8.5 mg, 0.0659 mmol, 2.0 eq). The mixture was stirred at room temperature for 1 h and quenched by HCOOH (0.5 mL), then purified by flash column chromatography to give 77 (49.2 mg, 0.0179 mmol, 55% yield) as a white solid. ESI-MS m/z: [M+H] calcd. for C₁₂₇H₁₈₈N₂₄O₄₂S 2753.30; found 1377.71.

### Example 48. Synthesis of tri-tert-butyl (13S, 17S)-1-(4-(((17S, 20S, 23S)-29-((5-((R)-5-(tert-butoxy)-2-((tert-butoxycarbonyl)amino)-5-oxopentyl)-2-hydroxyphenyl)amino)-17-(4-(2, 5-dioxo-2, 5-dihydro-1H-pyrrol-1-yl) butanamido)-20-isopropyl-23-methyl-9, 14, 18, 21, 24, 29-hexaoxo-3, 6-dioxa-10, 1312, 19, 22, 25-pentaazanonacosyl) carbamoyl)phenyl)-1, 10, 15-trioxo-2, 9, 14, 16-tetraazanonadecane-13, 17, 19-tricarboxylate (80)

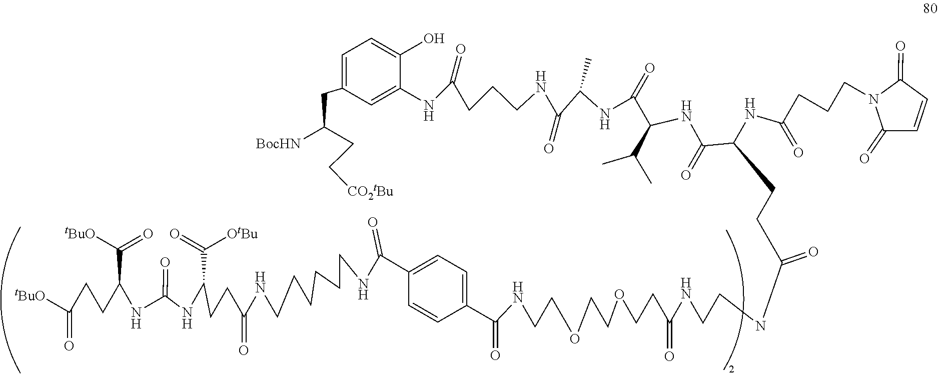

To a stirred solution of 69 (130 mg, 0.056 mmol) in DCM (8 mL) at room temperature were added 79 (32 mg, 0.067 mmol), HATU (28.0 mg, 0.073 mmol) and DIEA (11 mg, 0.08 mmol). The reaction mixture was stirred at room temperature for 0.5 h, diluted with DCM (50 mL), washed with water (40 mL) and brine (40 mL), concentrated. The residue was purified by a silica gel column (eluted with DCM and methanol), further purified by prep-HPLC, lyophilized to afford 80 (110 mg, 71% yield) as a white solid.

### Example 49. Synthesis of (13S, 17S)-1-(4-(((17S, 20S, 23S)-29-((5-((R)-2-amino-4-carboxybutyl)-2-hydroxyphenyl)amino)-17-(4-(2, 5-dioxo-2, 5-dihydro-1H-pyrrol-1-yl) butanamido)-20-isopropyl-23-methyl-9, 14, 18, 21, 24, 29-hexaoxo-3, 6-dioxa-10, 1312, 19, 22, 25-pentaazanonacosyl) carbamoyl)phenyl)-1, 10, 15-trioxo-2, 9, 14, 16-tetraazanonadecane-13, 17, 19-tricarboxylic acid (81)

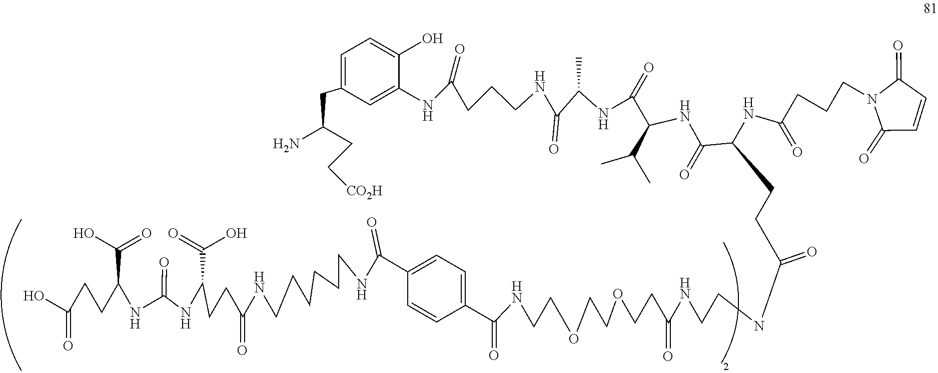

To a stirred solution of 80 (57 mg, 0.021 mmol) in DCM (1 mL) at room temperature was added TFA (6 mL). The reaction mixture was stirred at room temperature for 2 h, concentrated to afford 81 (110 mg, 71% yield) as a colorless oil.

### Example 50. Synthesis of (13S, 17S)-1-(4-(((17S, 20S, 23S)-29-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-17-(4-(2, 5-dioxo-2, 5-dihydro-1H-pyrrol-1-yl) butanamido)-20-isopropyl-23-methyl-9, 14, 18, 21, 24, 29-hexaoxo-3, 6-dioxa-10, 1312, 19, 22, 25-pentaazanonacosyl) carbamoyl)phenyl)-1, 10, 15-trioxo-2, 9, 14, 16-tetraazanonadecane-13, 17, 19-tricarboxylic acid (82)

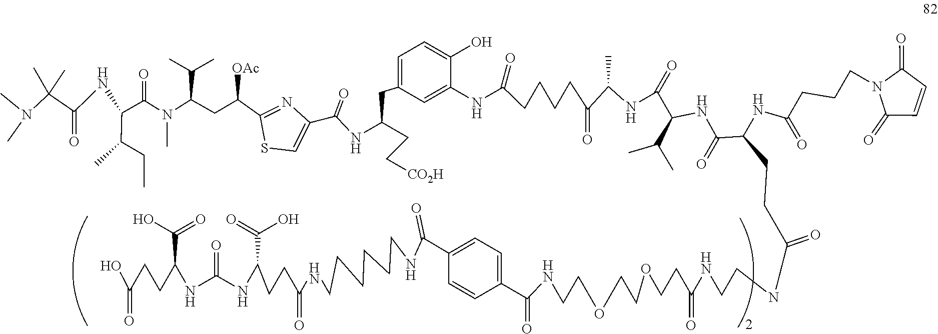

To a stirred solution of 81 (47 mg, 0.021 mmol) in DMF (5 mL) at room temperature were added DIEA (45 mg, 0.124 mmol) and 24 (15 mg, 0.021 mmol), the reaction mixture was stirred at room temperature for 2 h, purified by prep-HPLC, lyophilized to afford 82 (15 mg, 26% yield) as a white solid.

### Example 51. Synthesis of tert-butyl (S)-5-(((S)-1-(benzyloxy)-3-methyl-1-oxobutan-2-yl)amino)-4-(((benzyloxy) carbonyl)amino)-5-oxopentanoate (84)

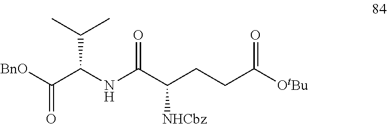 [SMILES: CC(C)[C@H](NC(=O)[C@H](CCC(=O)OC(C)(C)C)NC(=O)OCc1ccccc1)C(=O)OCc1ccccc1]

To a solution of compound 58 (10.00 g, 29.641 mmol, 1.0 eq), HATU (13.52 g, 35.569 mmol, 1.2 eq) and compound 2 (7.22 g, 29.641 mmol, 1.0 eq) in DCM (100 mL) was added DIEA (7.66 g, 59.282 mmol, 2.0 eq). The reaction mixture was stirred at room temperature for 2 h, and concentrated under reduced pressure. The residue was dissolved in ethyl acetate (100 mL), then washed with saturated NaHCO₃ (20 mL×3), 1 N HCl (20 mL×3), water (20 mL×3) and brine (10 mL×3), dried over anhydrous sodium sulfate, filtered and concentrated under reduced pressure to give compound 84 (17.00 g, 32.281 mmol, 109% yield) as a light brown oil. ESI-MS m/z: [M+H]⁺calcd. for C₂₉H₃₈N₂O₇ 526.27; found 527.31.

### Example 52. Synthesis of (S)-5-(((S)-1-(benzyloxy)-3-methyl-1-oxobutan-2-yl)amino)-4-(((benzyloxy) carbonyl)amino)-5-oxopentanoic acid (85)

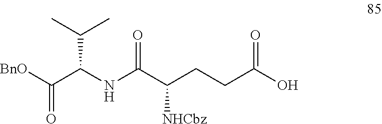 [SMILES: CC(C)[C@H](NC(=O)[C@H](CCC(=O)O)NC(=O)OCc1ccccc1)C(=O)OCc1ccccc1]

To a solution of compound 84 (17.00 g, 32.281 mmol) in DCM (20 mL) was added TFA (10 mL). The mixture was stirred at room temperature for 2 h and concentrated under reduced pressure. The residue was recrystallized from ethyl acetate/petroleum ether (1:1), to give 85 (13.00 g, 27.629 mmol, 85% yield) as a yellow solid. ESI-MS m/z: [M+H]⁺calcd. for C₂₅H₃₀N₂O₇ 470.21; found 471.31.

### Example 53. Synthesis of benzyl N2-((benzyloxy) carbonyl)-N5, N5-bis(2-((tert-butoxycarbonyl)amino) ethyl)-L- glutaminyl-L-valyl-L-alaninate (86)

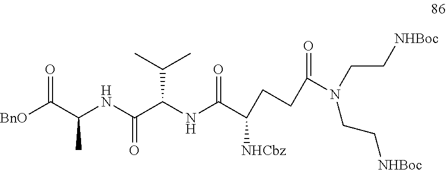 [SMILES: CC(C)[C@H](NC(=O)[C@H](CCC(=O)N(CCNC(=O)OC(C)(C)C)CCNC(=O)OC(C)(C)C)NC(=O)OCc1ccccc1)C(=O)N[C@@H](C)C(=O)OCc1ccccc1]

To a solution of compound 85 (4.00 g, 8.501 mmol, 1.0 eq) in DMF (40 mL) was added HATU (3.88 g, 10.201 mmol, 1.2 eq). The mixture was stirred at room temperature for 5 min. Then compound 61 (2.58 g, 8.501 mmol, 1.0 eq) and DIEA (2.75 g, 21.253 mmol, 2.5 eq) were added. The reaction mixture was stirred at room temperature for 0.5 h and then concentrated. The residue was dissolved in ethyl acetate (100 mL), then washed with saturated NaHCO₃ (20 mL×3), 1 N HCl (20 mL×3), water (20 mL×3) and brine (10 mL×3), dried over anhydrous sodium sulfate, filtered and concentrated under reduced pressure to give compound 86 (5.89 g, 7.792 mmol, 92% yield) as a light-yellow foam. ESI-MS m/z: [M+H]⁺ calcd. for C₃₉H₅₇N₅O₁₀ 755.41; found 756.52.

### Example 54. Synthesis of benzyl N5, N5-bis(2-aminoethyl)-N2-((benzyloxy) carbonyl)-L-glutaminyl-L-valinate (87)

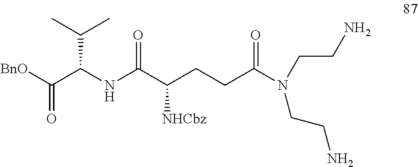 [SMILES: CC(C)[C@H](NC(=O)[C@H](CCC(=O)N(CCN)CCN)NC(=O)OCc1ccccc1)C(=O)OCc1ccccc1]

To a solution of compound 86 (5.89 g, 7.792 mmol) in DCM (30 mL) was added TFA (10 mL). The mixture was stirred at room temperature for 1.5 h and concentrated under reduced pressure. The residue was washed with petroleum ether (20 mL) and MTBE (20 mL), dried under reduced pressure to give compound 7 (5.50 g, 9.898 mol, 127% yield) as a yellow oil. ESI-MS m/z: [M+H]⁺calcd. for C₂₉H₄₁N₅O₆ 555.31; found 556.35.

### Example 55. Synthesis of benzyl (19S, 22S)-19-(((benzyloxy) carbonyl)amino)-1-(4-(tert-butoxycarbonyl)phenyl)-22-isopropyl-1, 11, 16, 20-tetraoxo-5, 8-dioxa-2, 12, 1512, 21-tetraazatricosan-23-oate (88)

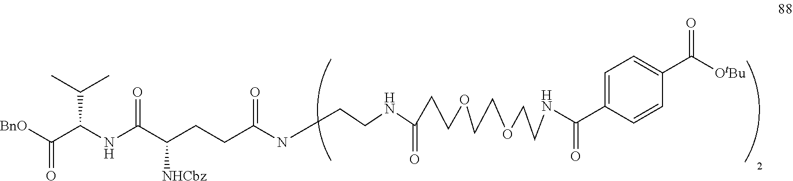

To a solution of compound 64 (5.00 g, 13.109 mmol, 2.2 eq) in DMF (5 mL) was added HATU (4.98 g, 13.109 mmol, 2.2 eq). The mixture was stirred at room temperature for 10 min. Then compound 87 (4.50 g, 5.959 mmol, 1.0 eq) and DIEA (34.8 mg, 0.269 mmol, 1.5 eq) were added. The reaction mixture was stirred at room temperature for 1 h, quenched by HCOOH (0.5 mL), and purified by flash column chromatography to give 88 (5.50 g, 4.288 mmol, 72% yield) as a white foam. ESI-MS m/z: [M+H]⁺ calcd. for C₆₇H₉₁N₇O₁₈ 1281.64; found 1282.71.

### Example 56. Synthesis of 4-(((4S, 7S)-7-(((benzyloxy) carbonyl)amino)-4-isopropyl-3, 6, 10, 15-tetraoxo-1-phenyl-2, 18, 21-trioxa-5, 1112, 14-triazatricosan-23-yl) carbamoyl)benzoic acid (89)

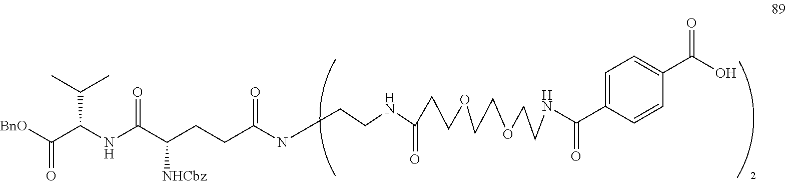

To a solution of compound 88 (3.50 g, 2.729 mmol) in DCM (7 mL) was added HCOOH (14 mL). The mixture was stirred at 50° C. for 16 h, concentrated under reduced pressure, and then purified by flash column chromatography to give 89 (1.10 g, 0.940 mmol, 34% yield) as a white foam. ESI-MS m/z: [M+H]+calcd. for C₅₉H₇₅N₇O₁₈ 1169.52; found 1170.61.

### Example 57. Synthesis of tri-tert-butyl (13S, 17S)-1-(4-(((4S, 7S)-7-(((benzyloxy) carbonyl)amino)-4-isopropyl-3, 6, 10, 15-tetraoxo-1-phenyl-2, 18, 21-trioxa-5, 1112, 14-triazatricosan-23-yl) carbamoyl)phenyl)-1, 10, 15-trioxo-2, 9, 14, 16-tetraazanonadecane-13, 17, 19-tricarboxylate (90)

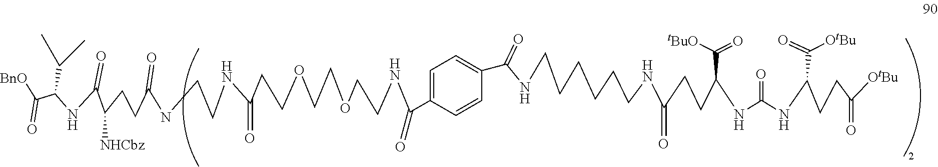

To a solution of compound 89 (740.0 mg, 0.632 mmol, 1.0 eq) in DMF (10 mL) was added HATU (553.0 mg, 1.454 mmol, 2.3 eq). The mixture was stirred at room temperature for 10 min. Then compound 16 (834.8 mg, 1.423 mmol, 2.25 eq) and DIEA (245.2 mg, 1.897 mmol, 3.0 eq) were added. The reaction mixture was stirred at room temperature for 0.5 h, and quenched by HCOOH (0.5 mL), then purified by flash column chromatography to give 90 (1.38 g, 0.598 mmol, 95% yield) as a white foam. ESI-MS m/z: [M+H]⁺ calcd. for C₁₁₇H₁₇₉N₁₅O₃₂ 2306.28; found 1154.21.

### Example 58. Synthesis of ((S)-19-amino-1-(4-(((7S, 11S)-7, 11-bis(tert-butoxycarbonyl)-2, 2-dimethyl-4, 9, 14-trioxo-3-oxa-8, 10, 15-triazahenicosan-21-yl) carbamoyl)phenyl)-1, 11, 16-trioxo-5, 8-dioxa-2, 12, 1512-triazaicosan-20-oyl)-L-valine (91)

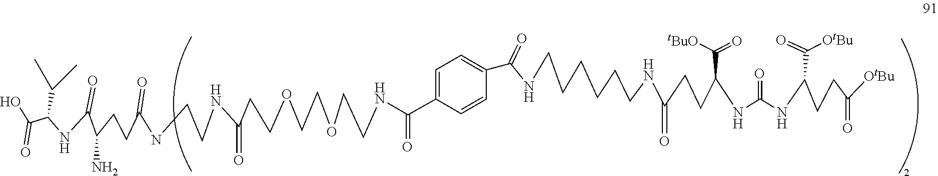

To a solution of compound 90 (1.38 g, 0.598 mmol) in PrOH (30 mL) was added Pd/C (0.72 g, 10 wt %) under N₂ atmosphere. The suspension was degassed and purged with H₂ for 3 times. The mixture was stirred under H₂ at room temperature for 16 h, and then filtered and concentrated to give compound 91 (1.21 g, 0.581 mmol, 97% yield) as a gray foam. ESI-MS m/z: [M+H]⁺ calcd. for C₁₀₂H₁₆₇N₁₅O₃₀ 2082.20; found 1042.21.

### Example 59. Synthesis of ((S)-1-(4-(((7S, 11S)-7, 11-bis(tert-butoxycarbonyl)-2, 2-dimethyl-4, 9, 14-trioxo-3-oxa-8, 10, 15-triazahenicosan-21-yl) carbamoyl)phenyl)-19-((4-(2, 5-dioxo-2, 5-dihydro-1H-pyrrol-1-yl)butyl)amino)-1, 11, 16-trioxo-5, 8-dioxa-2, 12, 1512-triazaicosan-20-oyl)-L-valine (92)

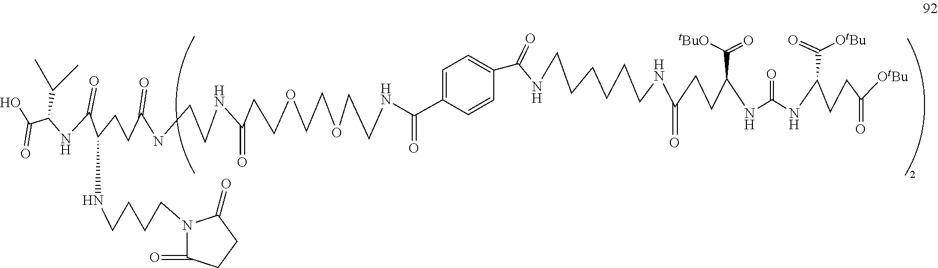

To a solution of compound 91 (1060.0 mg, 0.509 mmol, 1.0 eq) and compound 19 (192.5 mg, 0.687 mmol, 1.35 mmol) in DMF (10 mL) was added DIEA (131.5 mg, 1.018 mmol, 2.0 eq). The mixture was stirred at room temperature for 1 h and concentrated under reduced pressure. The residue was purified by flash column chromatography to give 92 (1.05 g, 0.0467 mmol, 92% yield) as a white solid. ESI-MS m/z: [M+H]⁺ calcd. for C₁₁₀H₁₇₄N₁₆O₃₃ 2247.24; found 1124.71.

### Example 60. Synthesis of tri-tert-butyl (13S, 17S)-1-(4-(((17S, 20S, 23S)-24-((5-((R)-5-(tert-butoxy)-2-((tert-butoxycarbonyl)amino)-5-oxopentyl)-2-hydroxyphenyl)amino)-17-(4-(2, 5-dioxo-2, 5-dihydro-1H-pyrrol-1-yl) butanamido)-20-isopropyl-23-methyl-9, 14, 18, 21, 24-pentaoxo-3, 6-dioxa-10, 1312, 19, 22-tetraazatetracosyl) carbamoyl)phenyl)-1, 10, 15-trioxo-2, 9, 14, 16-tetraazanonadecane-13, 17, 19-tricarboxylate (93)

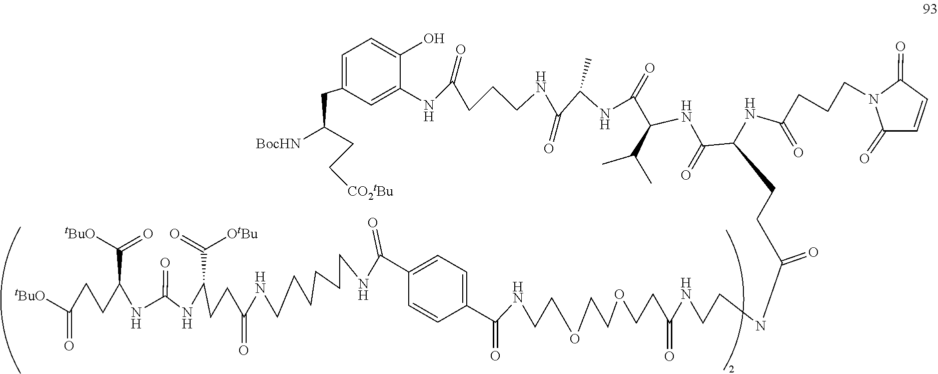

To a solution of compound 92 (290.0 mg, 0.129 mmol, 1.0 eq) in DMF (10 mL) was added HATU (51.5 mg, 0.135 mmol, 1.05 eq). The mixture was stirred at room temperature for 10 min. Then compound 21 (58.2 mg, 0.129 mmol, 1.0 eq) and DIEA (21.7 mg, 0.168 mmol, 1.3 eq) were added. The reaction mixture was stirred at room temperature for 0.5 h, quenched by HCOOH (0.5 mL), and purified by flash column chromatography to give 93 (187.0 mg, 0.0697 mmol, 53% yield) as a white solid. ESI-MS m/z: [M+H]⁺ calcd. for C₁₃₃H₂₀₉N₁₉O₃₈ 2680.50; found 1341.31.

### Example 61. Synthesis of (13S, 17S)-1-(4-(((17S, 20S, 23S)-24-((5-((R)-2-amino-4-carboxybutyl)-2-hydroxyphenyl)amino)-17-(4-(2, 5-dioxo-2, 5-dihydro-1H-pyrrol-1-yl) butanamido)-20-isopropyl-23-methyl-9, 14, 18, 21, 24-pentaoxo-3, 6-dioxa-10, 1312, 19, 22-tetraazatetracosyl) carbamoyl)phenyl)-1, 10, 15-trioxo-2, 9, 14, 16-tetraazanonadecane-13, 17, 19-tricarboxylic acid (94)

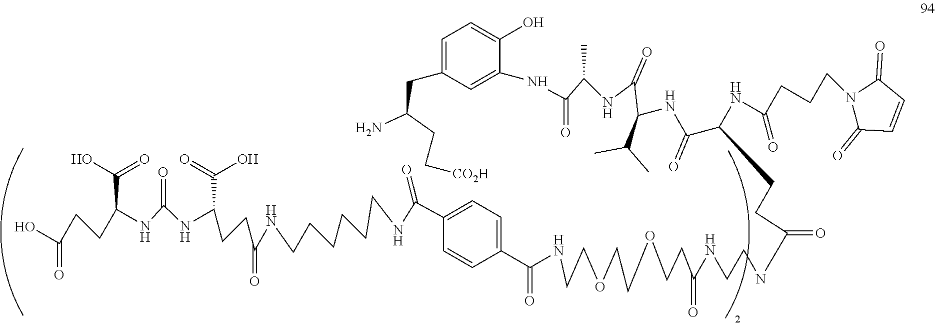

To a solution of compound 93 (187.0 mg, 0.0697 mmol) in DCM (2 mL) was added TFA (2 mL). The mixture was stirred at room temperature for 0.5 h and concentrated under reduced pressure, washed with petroleum ether (5 mL), dried under reduced pressure to give 94 (153 mg, 0.0699 mmol, 100% yield) as a light-yellow foam. ESI-MS m/z: [M+H]⁺calcd. for C₁₀₀H₁₄₅N₁₉O₃₆ 2188.01; found 1095.10.

### Example 62. Synthesis of (13S, 17S)-1-(4-(((17S, 20S, 23S)-24-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-17-(4-(2, 5-dioxo-2, 5-dihydro-1H-pyrrol-1-yl) butanamido)-20-isopropyl-23-methyl-9, 14, 18, 21, 24-pentaoxo-3, 6-dioxa-10, 1312, 19, 22-tetraazatetracosyl) carbamoyl)phenyl)-1, 10, 15-trioxo-2, 9, 14, 16-tetraazanonadecane-13, 17, 19-tricarboxylic acid (95)

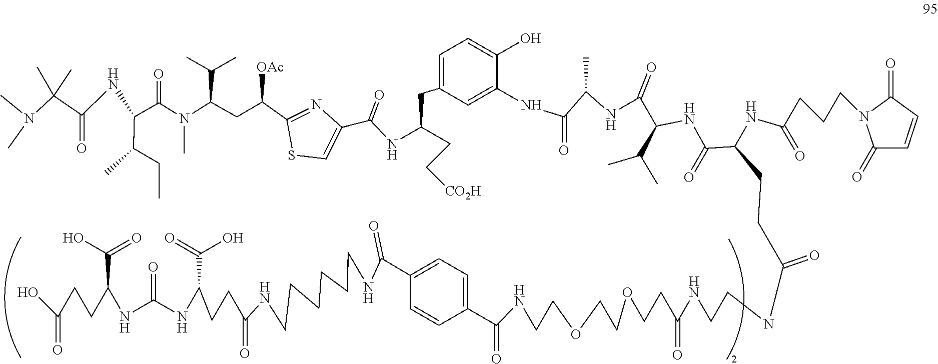

To a solution of compound 94 (133.0 mg, 0.0607 mmol, 1.0 eq) and compound 24 (48.4 mg, 0.0699 mmol, 1.15 mmol) in DMF (3 mL) was added DIEA (23.6 mg, 0.182 mmol, 3.0 eq). The mixture was stirred at room temperature for 2.5 h and quenched by HCOOH (0.5 mL), then purified by flash column chromatography to give 95 (48.6 mg, 0.0180 mmol, 30% yield) as a white solid. ESI-MS m/z: [M +H]⁺ calcd. for C₁₂₅H₁₈₅N₂₃O₄₁S 2696.28; found 1349.20.

### Example 63. Synthesis of hexa-tert-butyl 1,1′-((15-((S)-5-(((S)-1-(((S)-1-(benzyloxy)-1-oxopropan-2-yl)amino)-3-methyl-1-oxobutan-2-yl)amino)-4-(((benzyloxy) carbonyl)amino)-5-oxopentanoyl)-11, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 7, 15-trioxo-2, 8, 14, 16-tetraazanonadecane-13, 17, 19-tricarboxylate) (97)

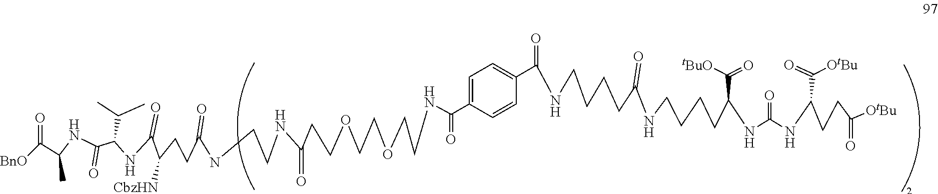

To a stirred solution of 66 (757.0 mg, 0.61 mmol) in DMF (10 mL) at room temperature were added 37 (895.0 mg, 1.52 mmol), HATU (697.0 mg, 1.83 mmol) and DIEA (355.0 mg, 2.74 mmol). The reaction mixture was stirred at room temperature for 0.5 h and then diluted with DCM (150 mL), washed with water (100 mL). The organic phase was concentrated, purified by a silica gel column, eluted with DCM and methanol, to afford 97 (1.04 g, 710% yield) as a white solid.

### Example 64. Synthesis of hexa-tert-butyl 1,1′-((15-((S)-5-(((S)-1-(((S)-1-oxopropan-2-yl)amino)-3-methyl-1-oxobutan-2-yl)amino)-4-((amino)-5-oxopentanoyl)-11, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 7, 15-trioxo-2, 8, 14, 16-tetraazanonadecane-13, 17, 19-tricarboxylate) (98)

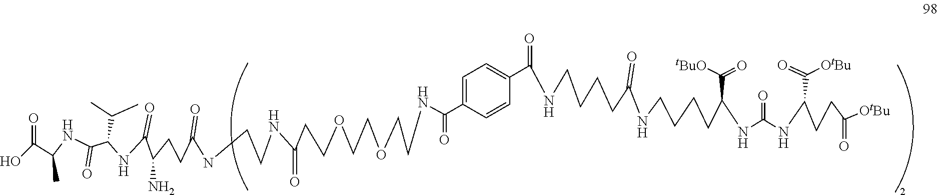

To a stirred solution of 97 (904.0 mg, 0.38 mmol) in isopropyl alcohol (10 mL) was added Pd/C (1.0 g, 10 wt %). The mixture was stirred overnight under a hydrogen balloon at room temperature and then filtered over celite. The filtrate was concentrated to give 98 (685 mg, 83% yield) as a brown solid.

### Example 65. Synthesis of hexa-tert-butyl 1,1′-((15-((S)-5-(((S)-1-(((S)-1-oxopropan-2-yl)amino)-3-methyl-1-oxobutan-2-yl)amino)-4-(((4-(2, 5-dioxo-2, 5-dihydro-1H-pyrrol-1-yl)butyl)amino)-5-oxopentanoyl)-11, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 7, 15-trioxo-2, 8, 14, 16-tetraazanonadecane-13, 17, 19-tricarboxylate) (99)

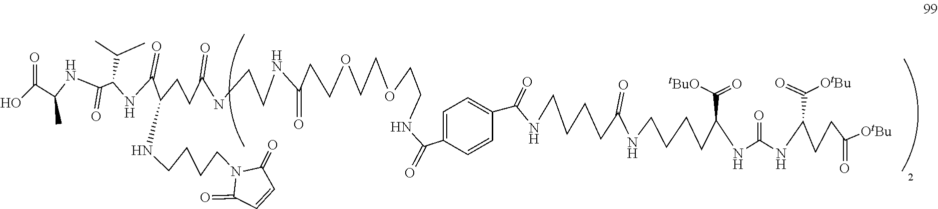

To a stirred solution of 98 (685 mg, 0.318 mmol) in DMF (10 mL) at room temperature were added 19 (135.0 mg, 0.48 mmol) and DIEA (84 mg, 0.64 mmol). The mixture was stirred for 2 h at room temperature and purified by prep-HPLC, lyophilized to afford 99 (435 mg, 57% yield) as a pink solid.

### Example 66. Synthesis of hexa-tert-butyl 1,1′-((15-((S)-5-(((S)-1-(((S)-1-((2-((5-((R)-5-(tert-butoxy)-2-((tert-butoxycarbonyl)amino)-5-oxopentyl)-2-hydroxyphenyl)amino)-2-oxoethyl)amino)-1-oxopropan-2-yl)amino)-3-methyl-1-oxobutan-2-yl)amino)-4-((4-(2, 5-dioxo-2, 5-dihydro-1H-pyrrol-1-yl)butyl)amino)-5-oxopentanoyl)-11, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 7, 15-trioxo-2, 8, 14, 16-tetraazanonadecane-13, 17, 19-tricarboxylate) (100)

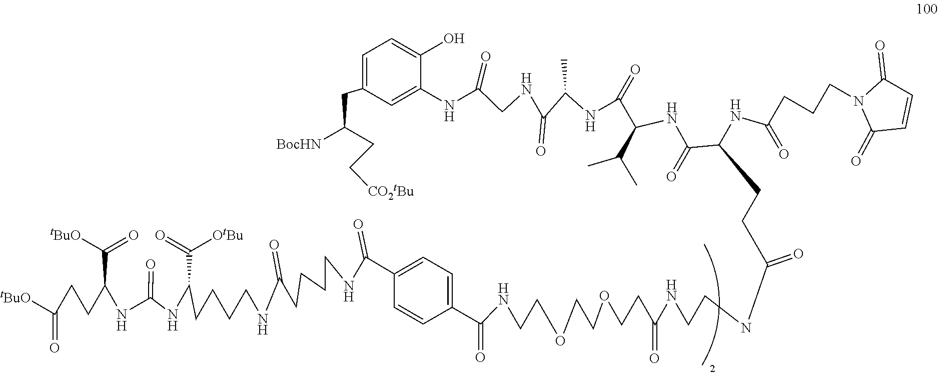 [SMILES: CC(C)[C@H](NC(=O)[C@H](CCC(=O)NCCNC(=O)CCOCCOCCNC(=O)c1ccc(C(=O)NCCCCC(=O)NCCCC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)N[C@@H](C)C(=O)NCC(=O)Nc1cc(C[C@@H](CCC(=O)OC(C)(C)C)NC(=O)OC(C)(C)C)ccc1O]

To a stirred solution of 99 (104.0 mg, 0.045 mmol) in DCM (10 mL) at room temperature were added 74 (19.0 mg, 0.045 mmol), HATU (19.0 mg, 0.049 mmol) and DIEA (9 mg, 0.067 mmol). The reaction mixture was stirred for 1 h at room temperature, diluted with DCM (50 mL), washed by water (50 mL) and brine (50 mL). The organic phase was concentrated in vacuo and the residue was purified by a silica gel column, eluted with DCM and methanol, to afford 100 (94 mg, 75% yield) as a colorless oil.

### Example 67. Synthesis of (13S, 13'S, 17S, 17'S)-1,1′-((15-((S)-5-(((S)-1-(((S)-1-((2-((5-((R)-2-amino-4-carboxybutyl)-2-hydroxyphenyl)amino)-2-oxoethyl)amino)-1-oxopropan-2-yl)amino)-3-methyl-1-oxobutan-2-yl)amino)-4-((4-(2, 5-dioxo-2, 5-dihydro-1H-pyrrol-1-yl)butyl)amino)-5-oxopentanoyl)-11, 19-dioxo-5, 8, 22, 25-tetraoxa-2, 12, 15, 18, 28-pentaazanonacosanedioyl)bis (4, 1-phenylene))bis (1, 7, 15-trioxo-2, 8, 14, 16-tetraazanonadecane-13, 17, 19-tricarboxylic acid) (101)

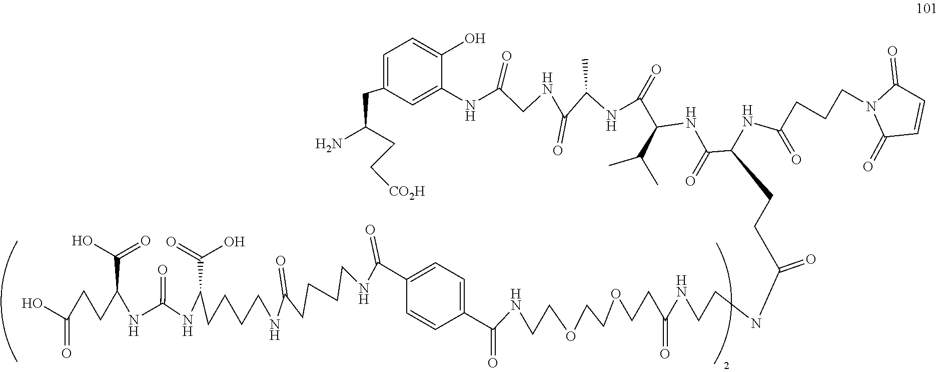

To a stirred solution of 100 (94.0 mg, 0.035 mmol) in DCM (1 mL) at room temperature was added TFA (6 mL). The mixture was stirred at room temperature for 2 h and concentrated in vacuo to afford 101 (77.0 mg, 100% yield) as a colorless oil.

### Example 68. Synthesis of (13S, 13'S, 17S, 17'S)-1,1′-((15-((S)-5-(((S)-1-(((S)-1-((2-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-2-oxoethyl)amino)-1-oxopropan-2-yl)amino)-3-methyl-1-oxobutan-2-yl)amino)-4-((4-(2, 5-dioxo-2, 5-dihydro-1H-pyrrol-1-yl)butyl)amino)-5-oxopentanoyl)-1 1, 19-dioxo-5, 8, 22, 25-tetraoxa-2, 12, 15, 18, 28-pentaazanonacosanedioyl)bis (4, 1-phenylene))bis (1, 7, 15-trioxo-2, 8, 14, 16-tetraazanonadecane-13, 17, 19-tricarboxylic acid) (102)

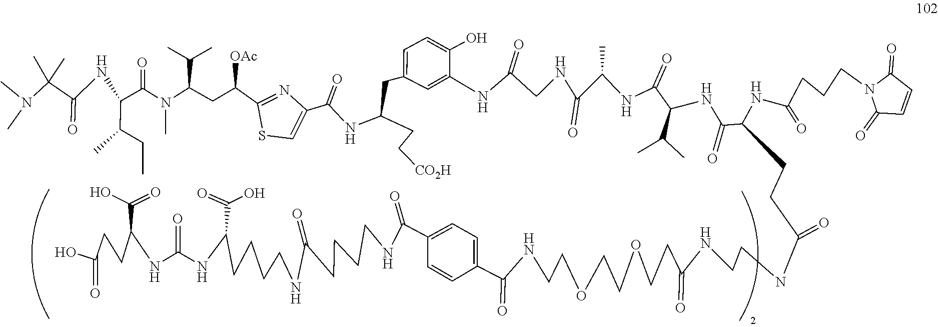

To a stirred solution of 101 (77.0 mg, 0.035 mmol) in DMF (1 mL) at room temperature were added 24 (25.0 mg, 0.035 mmol) and DIEA (23.0 mg, 0.17 mmol). The mixture was stirred at room temperature for 2 h and purified by prep-HPLC, lyophilized to afford 102 (21 mg, 210% yield) as a white solid.

### Example 69. Synthesis of hexa-tert-butyl 1, 1′-((15-((S)-5-(((S)-1-(((S)-1-oxopropan-2-yl)amino)-3-methyl-1-oxobutan-2-yl)amino)-4-(((2-(2, 5-dioxo-2, 5-dihydro-1H-pyrrol-1-yl))butyl)amino)-5-oxopentanoyl)-11, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 7, 15-trioxo-2, 8, 14, 16-tetraazanonadecane-13, 17, 19-tricarboxylate) (104)

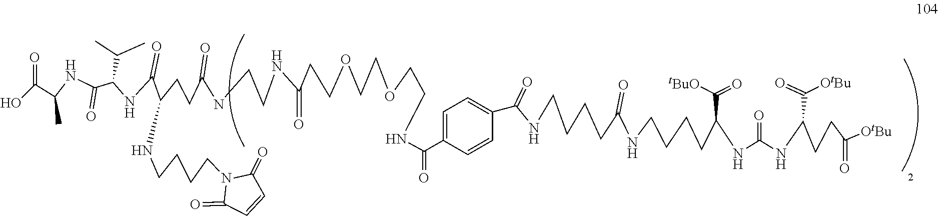

To a stirred solution of 98 (685 mg, 0.318 mmol) in DMF (10 mL) at room temperature were added 19b (119.0 mg, 0.48 mmol) and DIEA (84 mg, 0.64 mmol). The mixture was stirred at room temperature for 2 h and purified by prep-HPLC, lyophilized to afford 104 (410 mg, 53% yield) as a pink solid.

### Example 70. Synthesis of hexa-tert-butyl 1,1′-((15-((S)-5-(((S)-1-(((S)-1-((2-((5-((R)-5-(tert-butoxy)-2-((tert-butoxycarbonyl)amino)-5-oxopentyl)-2-hydroxyphenyl)amino)-2-oxoethyl)amino)-1-oxopropan-2-yl)amino)-3-methyl-1-oxobutan-2-yl)amino)-4-(2-(2, 5-dioxo-2, 5-dihydro-1H-pyrrol-1-yl) acetamido)-5-oxopentanoyl)-1 1, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 7, 15-trioxo-2, 8, 14, 16-tetraazanonadecane-13, 17, 19-tricarboxylate) (105)

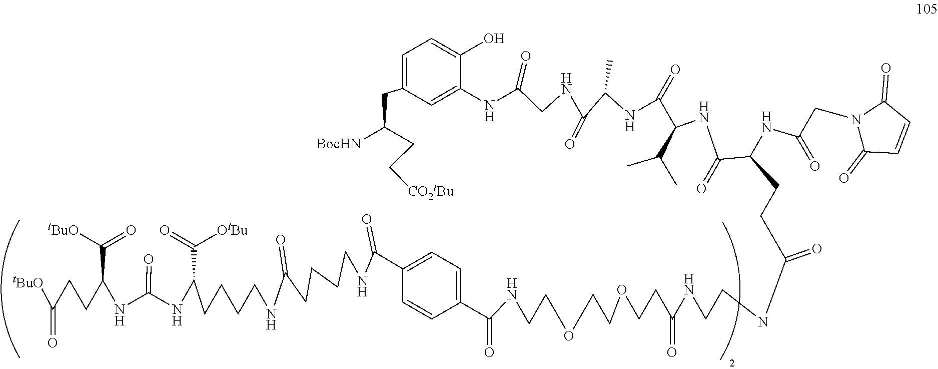

To a stirred solution of 104 (104.0 mg, 0.045 mmol) in DCM (10 mL) at room temperature were added 74 (19.0 mg, 0.045 mmol), HATU (19.0 mg, 0.049 mmol) and DIEA (9 mg, 0.067 mmol). The reaction mixture was stirred at room temperature for 1 h, diluted with DCM (50 mL), washed by water (50 mL) and brine (50 mL). The organic phase was concentrated in vacuo, purified by a silica gel column, eluted with DCM and methanol, to afford 105 (98 mg, 78% yield) as a colorless oil.

### Example 71. Synthesis of (13S, 13'S, 17S, 17'S)-1,1′-((15-((S)-5-(((S)-1-(((S)-1-((2-((5-((R)-2-amino-4-carboxybutyl)-2-hydroxyphenyl)amino)-2-oxoethyl)amino)-1-oxopropan-2-yl)amino)-3-methyl-1-oxobutan-2-yl)amino)-4-(2-(2, 5-dioxo-2, 5-dihydro-1H-pyrrol-1-yl) acetamido)-5-oxopentanoyl)-11, 19-dioxo-5, 8, 22, 25-tetraoxa-2, 12, 15, 18, 28-pentaazanonacosanedioyl)bis (4, 1-phenylene))bis (1, 7, 15-trioxo-2, 8, 14, 16-tetraazanonadecane-13, 17, 19-tricarboxylic acid) (106)

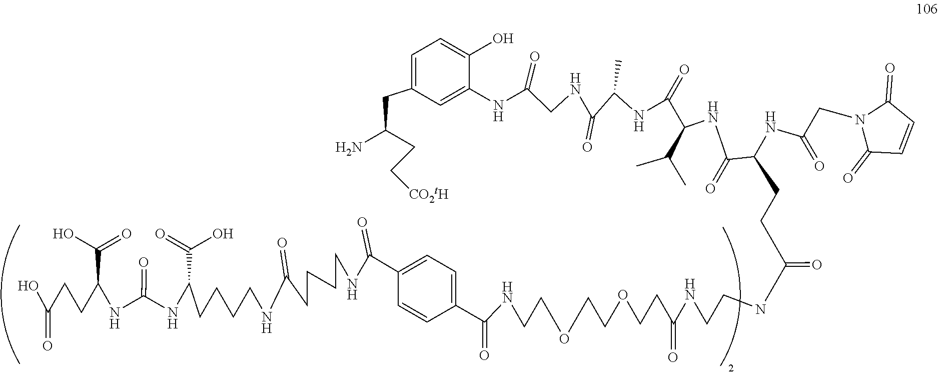

To a stirred solution of 105 (98.0 mg, 0.035 mmol) in DCM (1 mL) at room temperature was added TFA (6 mL). The mixture was stirred at room temperature for 2 h, concentrated in vacuo to afford 106 (81.0 mg, 100% yield) as a colorless oil.

### Example 72. Synthesis of (13S, 13'S, 17S, 17'S)-1,1′-((15-((S)-5-(((S)-1-(((S)-1-((2-((5-((R)-2-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-4-carboxybutyl)-2-hydroxyphenyl)amino)-2-oxoethyl)amino)-1-oxopropan-2-yl)amino)-3-methyl-1-oxobutan-2-yl)amino)-4-(2-(2, 5-dioxo-2, 5-dihydro-1H-pyrrol-1-yl) acetamido)-5-oxopentanoyl)-11, 19-dioxo-5, 8, 22, 25-tetraoxa-2, 12, 15, 18, 28-pentaazanonacosanedioyl)bis (4, 1-phenylene))bis (1, 7, 15-trioxo-2, 8, 14, 16-tetraazanonadecane-13, 17, 19-tricarboxylic acid) (107)

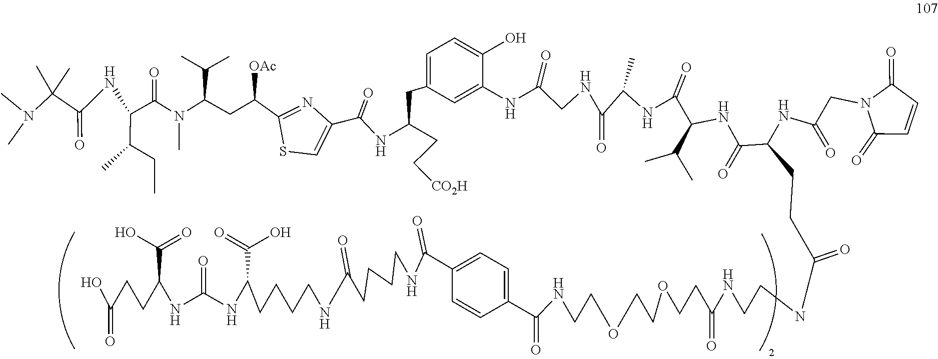

To a stirred solution of 106 (81.0 mg, 0.035 mmol) in DMF (1 mL) at room temperature were added 24 (25.0 mg, 0.035 mmol) and DIEA (23.0 mg, 0.17 mmol). The mixture was stirred for 2 h at room temperature, and purified by prep-HPLC, lyophilized to afford 107 (22 mg, 22% yield) as a white solid.

### Example 73. allyl (S)-(1-((4-(hydroxymethyl)phenyl)amino)-1-oxopropan-2-yl) carbamate (110)

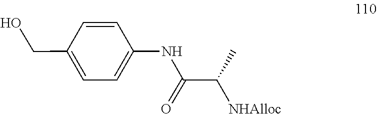

To a stirred solution of 109 (15.51 g, 89.565 mmol, 1.40 eq) in MeOH (90 mL) and DCM (180 mL) at room temperature was added EEDQ (31.64 g, 127.950 mmol, 2.00 eq). The resulting mixture was stirred at room temperature for 1 h before (4-aminophenyl) methanol (7.88 g, 63.975 mmol, 1.00 eq) was added. The resulting mixture was stirred at room temperature for further 12 h before it was concentrated in vacuo. Flash column chromatography (silica gel, petroleum ether: ethyl acetate=1:1 to DCM: MeOH=10:1) afforded 110 (4.00 g, 22% yield) as a white solid.

### Example 74 Synthesis of allyl (S)-(1-((4-((((4-nitrophenoxy) carbonyl)oxy)methyl)phenyl)amino)-1-oxopropan-2-yl) carbamate (111)

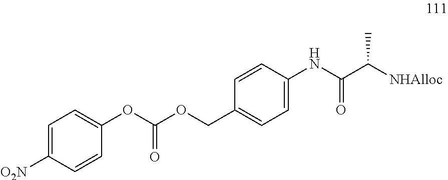

To a stirred solution of 110 (1.11 g, 3.661 mmol, 1.00 eq) in THF (100 mL) at room temperature were added 4-nitrophenyl chloroformate (1.11 g, 5.492 mmol, 1.50 eq) and pyridine (0.59 mL, 7.323 mmol, 2.00 eq) in sequence. The resulting mixture was stirred at room temperature for 2 h before it was quenched with NaHCO₃ (20 mL). The layers were separated, and the aqueous layer was extracted with ethyl acetate (3×30 mL). The combined organic layers were dried over anhydrous sodium sulfate and concentrated in vacuo. Flash column chromatography (silica gel, petroleum ether: ethyl acetate=2:1) afforded 111 (1.62 g, 100% yield) as a yellow solid.

### Example 75. Synthesis of 4-amino-1-((2R, 4S, 5R)-4-((tert-butyldimethylsilyl)oxy)-5-(((tert-butyldimethylsilyl)oxy)methyl) tetrahydrofuran-2-yl)pyrimidin-2 (1H)-one (112)

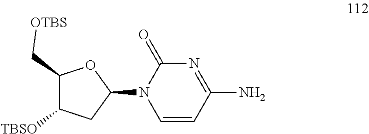 [SMILES: CC(C)(C)[Si](C)(C)OC[C@H]1O[C@@H](n2ccc(N)nc2=O)C[C@@H]1O[Si](C)(C)C(C)(C)C]

To a stirred solution of gemcitabine (5.00 g, 18.997 mmol, 1.00 eq) in DMF (150 mL) at room temperature were added imidazole (5.17 g, 75.988 mmol, 4.00 eq), Et₃N (5.27 mL, 37.994 mmol, 2.00 eq) and TBSCI (11.45 g, 75.988 mmol, 4.00 eq) in sequence. The resulting mixture was stirred at room temperature for 18 h before it was quenched with NaHCO₃ (50 mL). The layers were separated, and the aqueous layer was extracted with DCM (3×30 mL). The combined organic layers were dried over anhydrous sodium sulfate and concentrated in vacuo. Flash column chromatography (silica gel, petroleum ether: ethyl acetate=10:1) afforded the title compound (9.34 g, 100% yield) as a colorless oil.

### Example 76. Synthesis of allyl ((S)-1-((4-((((1-((2R, 4R, 5R)-4-((tert-butyldimethylsilyl)oxy)-5-(((tert-butyldimethylsilyl)oxy)methyl)-3, 3-difluorotetrahydrofuran-2-yl)-2-oxo-1, 2-dihydropyrimidin-4-yl) carbamoyl)oxy)methyl)phenyl)amino)-1-oxopropan-2-yl) carbamate (113)

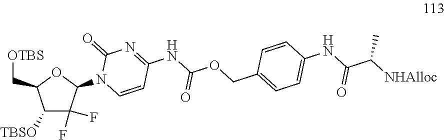

To a stirred solution of 112 (1.63 g, 3.321 mmol, 1.00 eq) in THF (80 mL) at −78° C. were added LHMDS (3.50 mL, 1.0 M in THF, 3.487 mmol, 1.05 eq) and a solution of 111 (1.62 g, 3.654 mmol, 1.10 eq) in THF (100 mL) in sequence. The resulting mixture was warmed to room temperature and stirred for 18 h before it was quenched with NH₄Cl (20 mL). The layers were separated, and the aqueous layer was extracted with ethyl acetate (3×30 mL). The combined organic layers were dried over anhydrous sodium sulfate and concentrated in vacuo. Flash column chromatography (silica gel, DCM: MeOH=20:1) afforded 113 (1.66 g, 63% yield) as a white solid.

### Example 77. Synthesis of 4-((S)-2-aminopropanamido)benzyl (1-((2R, 4R, 5R)-4-((tert-butyldimethylsilyl)oxy)-5-(((tert-butyldimethylsilyl)oxy)methyl)-3, 3-difluorotetrahydrofuran-2-yl)-2-oxo-1, 2-dihydropyrimidin-4-yl) carbamate (114)

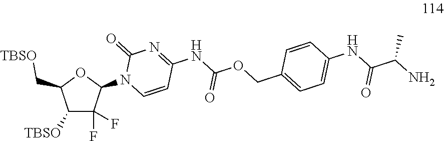 [SMILES: C[C@H](N)C(=O)Nc1ccc(COC(=O)Nc2ccn([C@@H]3O[C@H](CO[Si](C)(C)C(C)(C)C)[C@@H](O[Si](C)(C)C(C)(C)C)C3(F)F)c(=O)n2)cc1]

To a stirred solution of 113 (2.40 g, 3.069 mmol, 1.00 eq) in DCM (120 mL) at 0° C. were added Pd (PPh₃)₄(0.35 g, 0.307 mmol, 0.10 eq) and pyrrolidine (2.95 mL, 6.138 mmol, 2.00 eq). The resulting mixture was warmed to room temperature and stirred for 1.5 h before it was concentrated in vacuo. Flash column chromatography (silica gel, DCM: MeOH=20:1) afforded 114 (1.70 g, 79% yield) as a yellow solid. ESI-MS m/z: calcd. for C₃₂H₅₂F₂N₅O₇Si₂ [M+H]⁺ 712.3368; found 712.3365.

### Example 78. Synthesis of 6-(((tert-butyldimethylsilyl)oxy)methyl)-2, 2, 3, 3, 9, 9, 10, 10-octamethyl-4, 8-dioxa-3, 9-disilaundecan-6-amine (116)

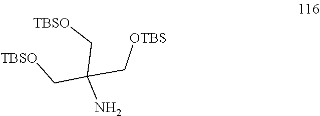 [SMILES: CC(C)(C)[Si](C)(C)OCC(N)(CO[Si](C)(C)C(C)(C)C)CO[Si](C)(C)C(C)(C)C]

To a stirred solution of 115 (6.00 g, 49.529 mmol, 1.00 eq) in DMF (50 mL) at room temperature were added imidazole (25.28 g, 371.471 mmol, 7.50 eq) and TBSCl (26.87 g, 178.306 mmol, 3.60 eq) in sequence. The resulting mixture was stirred at room temperature for 18 h before it was quenched with aq. NaHCO₃ (20 mL). The layers were separated, and the aqueous layer was extracted with DCM (3×50 mL). The combined organic layers were dried over anhydrous sodium sulfate and concentrated in vacuo. Flash column chromatography (silica gel, petroleum ether: ethyl acetate=4:1) afforded 116 (22.98 g, 100% yield) as a colorless oil.

### Example 79. Synthesis of allyl (6-(((tert-butyldimethylsilyl)oxy)methyl)-2, 2, 3, 3, 9, 9, 10, 10-octamethyl-4, 8-dioxa-3, 9-disilaundecan-6-yl) carbamate (117)

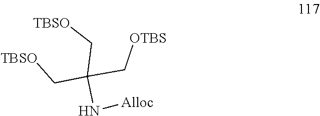

To a stirred solution of 116 (8.65 g, 18.645 mmol, 1.00 eq) in DCM (150 mL) at room temperature were added DMAP (2.28 g, 18.645 mmol, 1.00 eq), pyridine (12.00 mL, 149.128 mmol, 8.00 eq) and AllocCl (7.93 mL, 74.580 mmol, 4.00 eq) in sequence. The resulting mixture was stirred at room temperature for 3 h before it was quenched with aq. NaHCO₃ (50 mL). The layers were separated, and the aqueous layer was extracted with DCM (3×30 mL). The combined organic layers were dried over anhydrous sodium sulfate and concentrated in vacuo. Flash column chromatography (silica gel, petroleum ether: ethyl acetate - 50:1) afforded 117 (8.21 g, 80% yield) as a colorless oil.

### Example 80. Synthesis of allyl (1, 3-dihydroxy-2-(hydroxymethyl) propan-2-yl) carbamate (118)

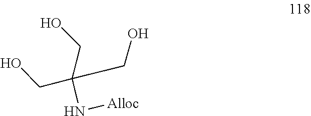

To a stirred solution of 117 (7.80 g, 14.234 mmol, 1.00 eq) in ethyl acetate (70 mL) at 0° C. was added HCl (2.0 M in ethyl acetate, 71.17 mL, 142.336 mmol, 10.00 eq). The resulting mixture was warmed to room temperature and stirred for 1.5 h before it was concentrated in vacuo directly. Flash column chromatography (silica gel, DCM: MeOH - 10:1) afforded 118 (2.00 g, 68% yield) as a colorless oil.

### Example 81. Synthesis of allyl (1, 3-bis(((4-nitrophenoxy) carbonyl)oxy)-2-((((4-nitrophenoxy) carbonyl)oxy)methyl) propan-2-yl) carbamate (69)

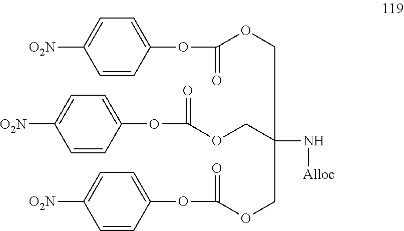

To a stirred solution of 118 (1.60 g, 8.000 mmol, 1.00 eq) in THF (70 mL) at room temperature were added 112 (7.07 g, 35.000 mmol, 4.50 eq) and pyridine (3.76 mL, 48.000 mmol, 6.00 eq) in sequence. The resulting mixture was stirred at room temperature for 2 h before it was concentrated in vacuo. Flash column chromatography (silica gel, petroleum ether: ethyl acetate - 2:1) afforded 119 (4.60 g, 82% yield) as a white solid.

### Example 82. Synthesis of di-tert-butyl (2S, 12S)-7-(((allyloxy) carbonyl)amino)-7-(((((S)-1-(tert-butoxy)-3-methyl-1-oxobutan-2-yl) carbamoyl)oxy)methyl)-2, 12-diisopropyl-4, 10-dioxo-5, 9-dioxa-3, 11-diazatridecanedioate (121)

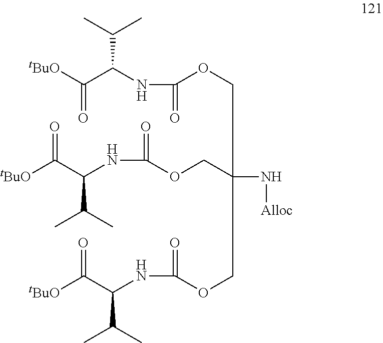

To a stirred solution of 119 (1.30 g, 1.856 mmol, 1.00 eq) in DMF (50 mL) at room temperature were added 120 (1.36 g, 6.495 mmol, 3.50 eq) and DIEA (1.74 mL, 9.993 mmol, 5.40 eq). The resulting mixture was stirred at room temperature for 12 h before it was quenched with brine (5 mL). The layers were separated, and the aqueous layer was extracted with DCM (3×5 mL). The combined organic layers were dried over anhydrous sodium sulfate and concentrated in vacuo. The crude 121 was obtained without further purification.

### Example 83. Synthesis of (2S, 12S)-7-(((allyloxy) carbonyl)amino)-7-(((((S)-1-carboxy-2-methylpropyl) carbamoyl)oxy)methyl)-2, 12-diisopropyl-4, 10-dioxo-5, 9-dioxa-3, 11-diazatridecanedioic acid (122)

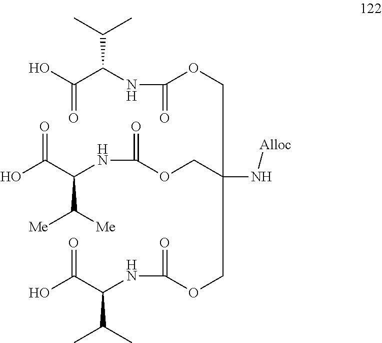

To a stirred solution of 121 in DCM (10 mL) at room temperature was added HCO₂H (30 mL). The resulting mixture was stirred at room temperature for 48 h before it was concentrated in vacuo. Prep-HPLC purification and lyophilization afforded 122 (0.99 g, 84% yield) as a colorless oil.

### Example 84. Synthesis of 2-(((allyloxy) carbonyl)amino)-2-(((((S)-1-(((S)-1-((4-((((1-((2R, 4R, 5R)-4-((tert-butyldimethylsilyl)oxy)-5-(((tert-butyldimethylsilyl)oxy)methyl)-3, 3-difluorotetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamoyl)oxy)methyl) propane-1, 3-diyl bis(((S)-1-(((S)-1-((4-((((1-((2R, 4R, 5R)-4-((tert-butyldimethylsilyl)oxy)-5-(((tert-butyldimethylsilyl)oxy)methyl)-3, 3-difluorotetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamate) (123)

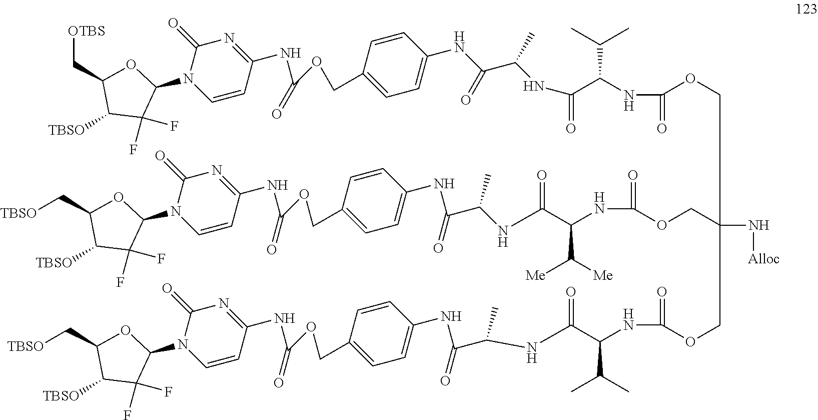

To a stirred solution of 122 (155.0 mg, 0.244 mmol, 1.00 eq) in DMF (3 mL) at room temperature were added HATU (287.9 mL, 0.757 mmol, 3.10 eq), DIEA (256 pL, 1.465 mmol, 6.00 eq) and a solution of 114 (573.8 mg, 0.806 mmol, 3.30eq) in DMF (4 mL) in sequence. The resulting mixture was stirred at room temperature for 2 h before it was quenched with brine (5 mL). The layers were separated, and the aqueous layer was extracted with DCM (3×5 mL). The combined organic layers were dried over anhydrous sodium sulfate and concentrated in vacuo. The crude product was obtained without further purification.

### Example 85. Synthesis of 2-amino-2-(((((S)-1-(((S)-1-((4-((((1-((2R, 4R, 5R)-4-((tert-butyldimethylsilyl)oxy)-5-(((tert-butyldimethylsilyl)oxy)methyl)-3, 3-difluorotetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamoyl)oxy)methyl) propane-1, 3-diyl bis(((S)-1-(((S)-1-((4-((((1-((2R, 4R, 5R)-4-((tert-butyldimethylsilyl)oxy)-5-(((tert-butyldimethylsilyl)oxy)methyl)-3, 3-difluorotetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamate) (124)

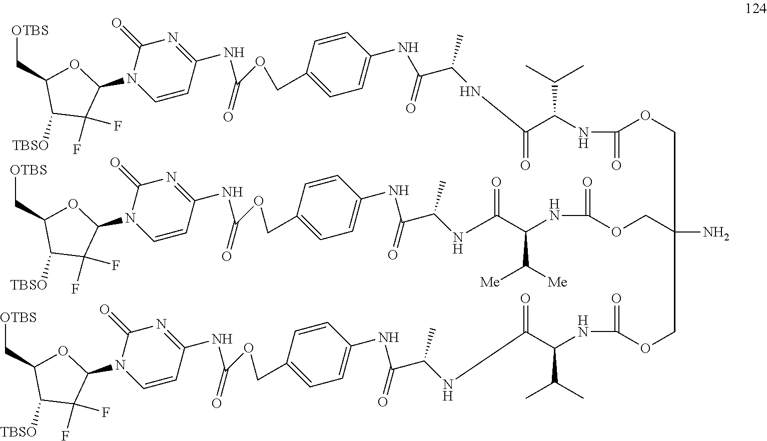 [SMILES: CC(C)[C@H](NC(=O)OCC(N)(COC(=O)N[C@H](C(=O)N[C@@H](C)C(=O)Nc1ccc(COC(=O)Nc2ccn([C@@H]3O[C@H](CO[Si](C)(C)C(C)(C)C)[C@@H](O[Si](C)(C)C(C)(C)C)C3(F)F)c(=O)n2)cc1)C(C)C)COC(=O)N[C@H](C(=O)N[C@@H](C)C(=O)Nc1ccc(COC(=O)Nc2ccn([C@@H]3O[C@H](CO[Si](C)(C)C(C)(C)C)[C@@H](O[Si](C)(C)C(C)(C)C)C3(F)F)c(=O)n2)cc1)C(C)C)C(=O)N[C@@H](C)C(=O)Nc1ccc(COC(=O)Nc2ccn([C@@H]3O[C@H](CO[Si](C)(C)C(C)(C)C)[C@@H](O[Si](C)(C)C(C)(C)C)C3(F)F)c(=O)n2)cc1]

To a stirred solution of 123 in DCM (5 mL) at 0° C. were added Pd (PPh₃) 4 (56.5 mg, 0.049 mmol, 0.20 eq) and pyrrolidine (82 pL, 0.977 mmol, 4.00 eq). The resulting mixture was warmed to room temperature and stirred for 1.5 h before it was concentrated in vacuo. The crude product 124 was obtained without further purification.

### Example 86. Synthesis of 2-amino-2-(((((S)-1-(((S)-1-((4-((((1-((2R, 4R, 5R)-3, 3-difluoro-4-hydroxy-5-(hydroxymethyl)tetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamoyl)oxy)methyl) propane-1, 3-diyl bis(((S)-1-(((S)-1-((4-((((1-((2R, 4R, 5R)-3, 3-difluoro-4-hydroxy-5-(hydroxymethyl)tetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamate) (125)

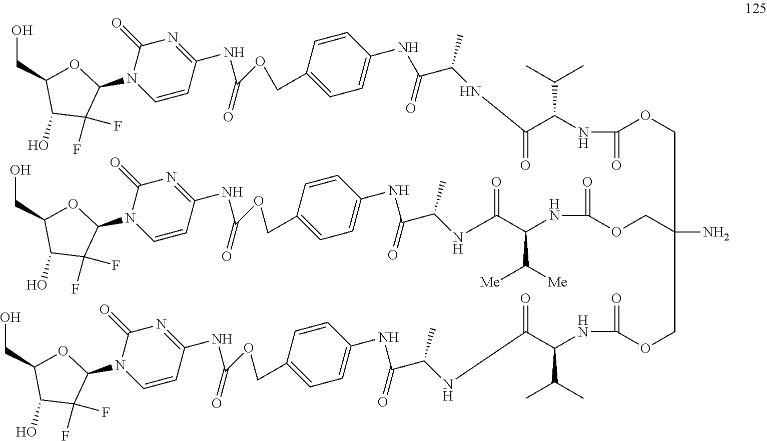 [SMILES: CC(C)[C@H](NC(=O)OCC(N)(COC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)COC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)C(=O)N[C@@H](C)C(=O)Nc1ccc(COC(=O)Nc2ccn([C@@H]3O[C@H](CO)[C@@H](O)C3(F)F)c(=O)n2)cc1]

To a stirred solution of 124 in THF (5 mL) at room temperature was added TBAF (0.74 mL, 1.0 M in THF, 0.732 mmol, 3.00 eq). The resulting mixture was stirred at room temperature for 0.5 h before it was quenched with MeOH (0.5 mL) and concentrated in vacuo. Prep-HPLC purification and lyophilization afforded 125 (230.0 mg, 48% yield over 3 steps) as a white solid.

### Example 87. rac-(37R, 45R)-45-(3-(tert-butoxy)-3-oxopropyl)-37-(4-(2, 5-dioxo-2, 5-dihydro-1H-pyrrol-1-yl) butanamido)-31, 38, 43-trioxo-2, 5, 8, 11, 14, 17, 20, 23, 26, 29-decaoxa-32, 39, 44-triazahexatetracontan-46-oic acid (127)

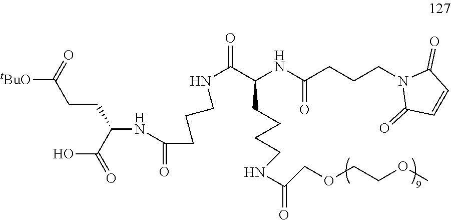

To a stirred solution of 126 (5.0 g, 5.78 mmol) in DCM (100 mL) at 0° C. were added NHS (0.73 g, 6.359 mmol) and EDC-HCl (2.22 g, 11.56 mmol). After 2 h, L- glutamic acid 5-tert-butyl ester (1.52 g, 7.50m mmol) was added, and the resulting mixture was stirred at r.t. for 18 h before it was quenched with NH₄Cl. The layers were separated, and the aqueous layer was extracted with DCM. The combined organic layers were dried over anhydrous sodium sulfate and concentrated in vacuo. The corresponding crude 127 (5.0 g, 95% yield) was obtained without further purification.

### Example 88. Synthesis of tert-butyl ((S)-1-(((S)-1-((2-(((S)-4, 11-diethyl-8-fluoro-4-hydroxy-3, 14-dioxo-3, 4, 12, 14-tetrahydro-IH-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamate (130)

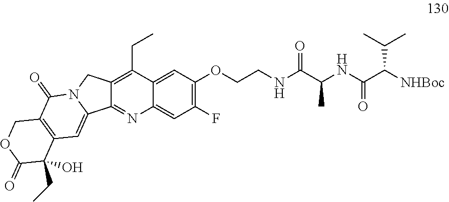 [SMILES: CCc1c2c(nc3cc(F)c(OCCNC(=O)[C@H](C)NC(=O)[C@@H](NC(=O)OC(C)(C)C)C(C)C)cc13)-c1cc3c(c(=O)n1C2)COC(=O)[C@]3(O)CC]

To a solution of 128 (2.00 g, 4.410 mmol, 1.0 eq), HATU (1.93 g, 5.072 mmol, 1.15 eq) and Boc-Val-Ala-OH (1.40 g, 4.851 mmol, 1.1 eq) in DCM (20 mL) were added, followed by DIEA (0.86 g, 6.616 mmol, 1.5 eq). The reaction mixture was stirred at room temperature for 3 h, washed with brine (5 mL×3), dried over anhydrous sodium sulfate, filtered and concentrated under reduced pressure to give 130 (4.00 g, 5.526 mmol, 125% yield) as a yellow foam. ESI-MS m/z: [M+H]⁺calcd. for C₃₇H₄₆FN₅O₉ 723.80; found 724.92.

### Example 89. Synthesis of (S)-2-amino-N—((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)-3-methylbutanamide (131)

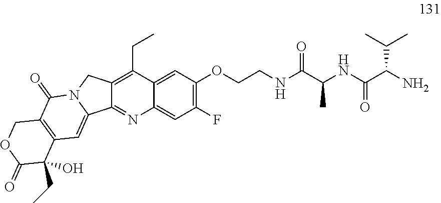 [SMILES: CCc1c2c(nc3cc(F)c(OCCNC(=O)[C@H](C)NC(=O)[C@@H](N)C(C)C)cc13)-c1cc3c(c(=O)n1C2)COC(=O)[C@]3(O)CC]

To a solution of 130 (4.00 g, 5.526 mmol) in DCM (10 mL) was added TFA (2 mL). The mixture was stirred at room temperature for 2 h and concentrated under reduced pressure. The residue was recrystallized from ethyl acetate to give 131 (1.71 g, 2.742 mmol, 49.57% yield) as a yellow solid. ESI-MS m/z: [M+H]⁺ calcd. for C₃₂H₃₈FN₅O₇ 623.28; found 624.31.

### Example 90. Synthesis of tert-butyl (S)-5-(((S)-1-(benzyloxy)-3-methyl-1-oxobutan-2-yl)amino)-4-(((benzyloxy) carbonyl)amino)-5-oxopentanoate (132)

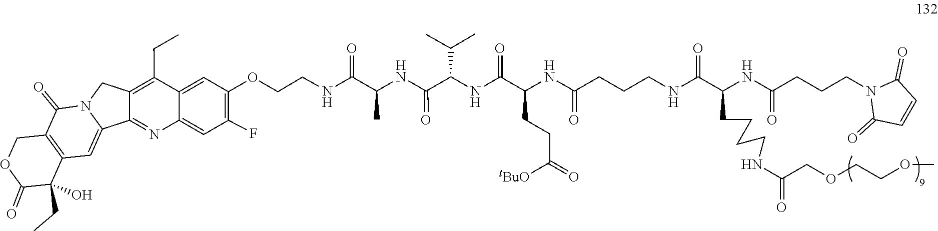

To a solution of compound 131 (1.06 g, 1.700 mmol, 1.0 eq) in DMF (15 mL) was added HATU (0.78 g, 2.040 mmol, 1.2 eq). The mixture was stirred at room temperature for 5 min. Then 127 (0.63 g, 1.870 mmol, 1.1 eq) and DIEA (0.44 g, 3.399 mmol, 2.0 eq) were added. The reaction mixture was stirred at room temperature for 0.5 h. The mixture was concentrated under reduced pressure. The residue was recrystallized from ethyl acetate: petroleum ether - 1:1 to give 132 (3.6 g, 2.33 mmol, 68% yield) as a yellow solid.

### Example 91. Synthesis of (13S, 21S)-21-(((S)-1-(((S)-1-((2-(((S)-4, 11-diethyl-8-fluoro-4-hydroxy-3, 14-dioxo-3, 4, 12, 14-tetrahydro-1H-pyrano [3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-13-(4-(2, 5-dioxo-2, 5-dihydro-1H-pyrrol-1-yl) butanamido)-7, 14, 19-trioxo-2, 5-dioxa-8, 15, 20-triazatetracosan-24-oic acid (133)

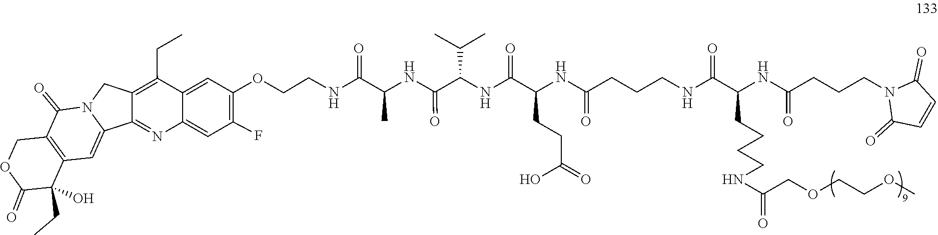

To a solution of 132 (3.6 g, 32.281 mmol) in DCM (20 mL) was added TFA (10 mL). The mixture was stirred at room temperature for 2 h and concentrated under reduced pressure. The residue was recrystallized from ethyl acetate: petroleum ether - 1:1 to give 133 (13.0 g, 27.6 mmol, 115% yield) as a yellow solid.

### Example 92. Synthesis of 2-((13S, 21S)-21-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-13-(4-(2, 5-dioxo-2, 5-dihydro-1H-pyrrol-1-yl) butanamido)-7, 14, 19-trioxo-2, 5-dioxa-8, 15, 20-triazatetracosan-24-amido)-2-(((((S)-1-(((S)-1-((4-((((1-((2R, 4R, 5R)-3, 3-difluoro-4-hydroxy-5-(hydroxymethyl)tetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamoyl)oxy)methyl) propane-1, 3-diyl bis(((S)-1-(((S)-1-((4-((((1-((2R, 4R, 5R)-3, 3-difluoro-4-hydroxy-5-(hydroxymethyl)tetrahydrofuran-2-yl)-2-oxo-1, 2-dihydropyrimidin-4-yl) carbamoyl)oxy)methyl)phenyl)amino)-1-oxopropan-2-yl)amino)-3-methyl-1-oxobutan-2-yl) carbamate) (134)

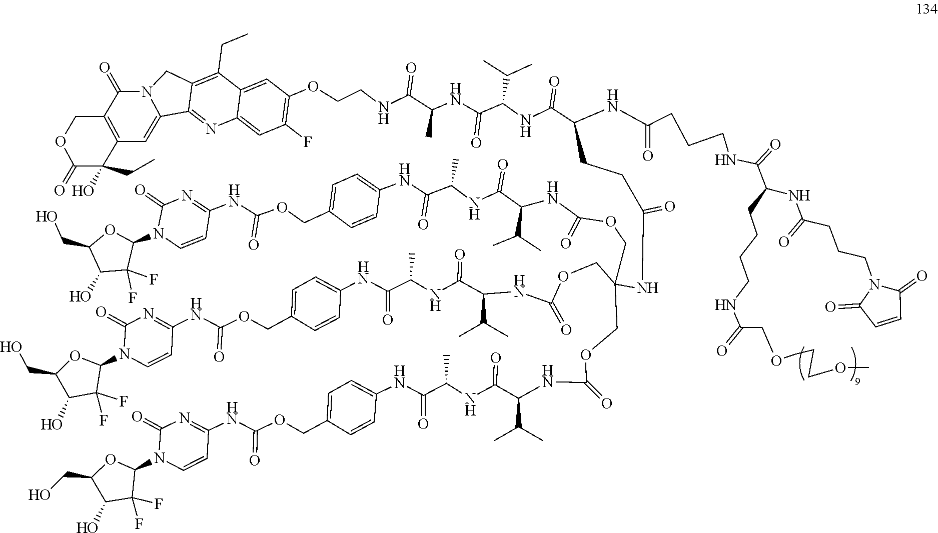

To a stirred solution of 133 (67.5 mg, 0.042 mmol, 1.00 eq) in DMF (1.5 mL) at room temperature were added HATU (16.0 mg, 0.042 mmol, 1.00 eq), 125 (85.1 mg, 0.042 mmol, 1.00 eq) and DIEA (15 μL, 0.084 mmol, 2.00 eq) in sequence. The resulting mixture was stirred at room temperature for 1 h. Prep-HPLC purification and lyophilization afforded 134 (22.0 mg, 14% yield) as a white solid. ESI-MS m/z: calcd. for C₁₅₅H₂₁₁F₇N₂₉O₅₅ [M+H]⁺ 3527.4488; found 3527.4490.

### Example 93. Synthesis of tert-butyl ((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamate (137)

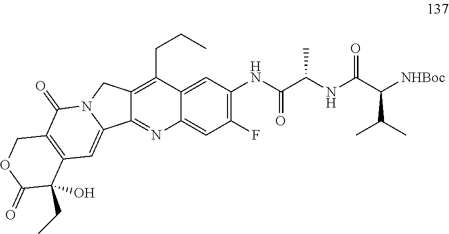 [SMILES: CCCc1c2c(nc3cc(F)c(NC(=O)[C@H](C)NC(=O)[C@@H](NC(=O)OC(C)(C)C)C(C)C)cc13)-c1cc3c(c(=O)n1C2)COC(=O)[C@]3(O)CC]

To a stirred solution of 136 (404 mg, 1.00 mmol, 1.00 eq) in DMF (1.5 mL) at room temperature were added HATU (418 mg, 1.1 mmol, 1.10 eq), Boc-Val-Ala-OH (200 mg, 1.00 mmol, 1.00 eq) and DIEA (258 mg, 2.00 mmol, 2.00 eq) in sequence. The resulting mixture was stirred at room temperature for 1 h. Prep-HPLC purification and lyophilization afforded 137 (540 mg, 80% yield) as a white solid.

### Example 94. Synthesis of (S)-2-amino-N—((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)-3-methylbutanamide (138)

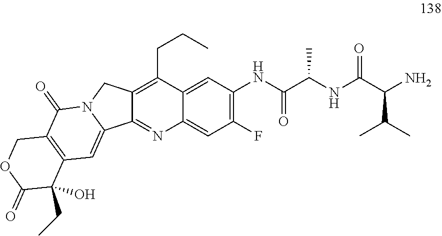 [SMILES: CCCc1c2c(nc3cc(F)c(NC(=O)[C@H](C)NC(=O)[C@@H](N)C(C)C)cc13)-c1cc3c(c(=O)n1C2)COC(=O)[C@]3(O)CC]

To a stirred solution of 137 (540 mg, 0.80 mmol) in DCM (20 mL) at room temperature was added TFA (20 mL). The resulting mixture was stirred at room temperature for 3 h, concentrated in vacuo to afford 138 (460 mg, 100.0% yield) as a white solid.

### Example 95. Synthesis of tert-butyl (37S, 45S)-37-(4-(2, 5-dioxo-2, 5-dihydro-1H-pyrrol-1-yl) butanamido)-45-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-31, 38, 43-trioxo-2, 5, 8, 11, 14, 17, 20, 23, 26, 29-decaoxa-32, 39, 44-triazaoctatetracontan-48-oate (139)

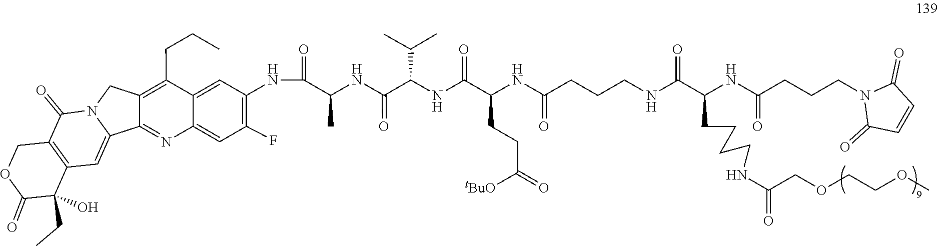

To a stirred solution of 138 (115 mg, 0.20 mmol) in DMF (1 mL) at room temperature were added 127 (210 mg, 0.20 mmol), HATU (91 mg, 0.24 mmol) and DIEA (65 mg, 0.50 mmol). The resulting mixture was stirred at room temperature for 30 min. before it was concentrated in vacuo. Prep-HPLC purification and lyophilization afforded 139 (274 mg, 75% yield).

### Example 96. Synthesis of (37S, 45S)-37-(4-(2, 5-dioxo-2, 5-dihydro-1H-pyrrol-1-yl) butanamido)-45-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-31, 38, 43-trioxo-2, 5, 8, 11, 14, 17, 20, 23, 26, 29-decaoxa-32, 39, 44-triazaoctatetracontan-48-oic acid (140)

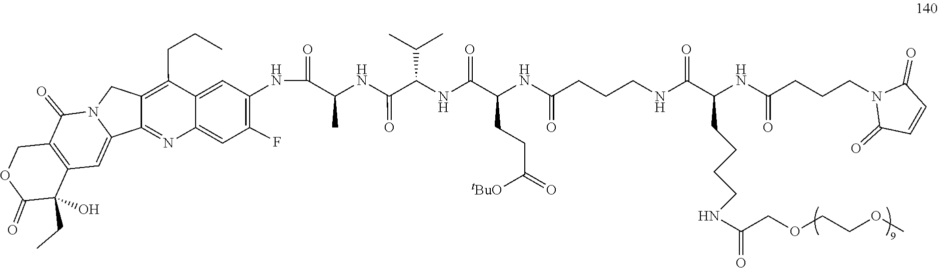

To a solution of 139 (2.3 g, 1.43 mmol) in DCM (10 mL) was added TFA (20 mL). The mixture was stirred at room temperature for 1 h and concentrated in vacuo. The residue was purified by prep-HPLC to afford 140 (1.2 g, 54% yield over two steps) as a yellow solid. ESI-MS m/z: calcd. for C₇₅H₁₁₀N₁₀O₀₂₅ [M+H]⁺ 1551.8; found 1551.8.

### Example 97. Synthesis of compound (141)

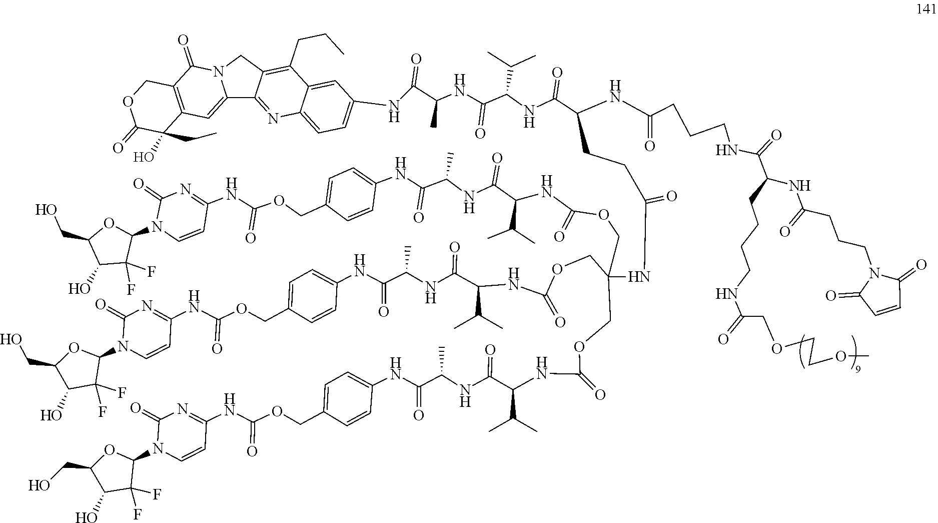

To a stirred solution of 140 (239.1 mg, 0.154 mmol, 1.50 eq) in DMF (5.0 mL) at room temperature were added HATU (58.6 mg, 0.154 mmol, 1.50 eq), 125 (200.0 mg, 0.103 mmol, 1.00 eq) and DIEA (51 μL, 0.308 mmol, 3.00 eq) in sequence. The resulting mixture was stirred at room temperature for 1 h before concentration. Prep-HPLC purification and lyophilization afforded 141 (143.0 mg, 40% yield) as a white solid. ESI-MS m/z: calcd. for C₁₅₇H₂₀₉F₆N₂₉O₅₄ [M+2H]²⁺ 1740.2202; found 1740.2202.

### Example 98. Synthesis of allyl ((S)-1-((4-((((S)-4,11-diethyl-8-fluoro-4-hydroxy-3,14-dioxo-3,4, 12,14-tetrahydro-1H-pyrano[3′, 4′:6, 7]indolizino[1,2-b]quinolin-9-yl)oxy)methyl)phenyl)amino)-1-oxopropan-2-yl) carbamate (144)

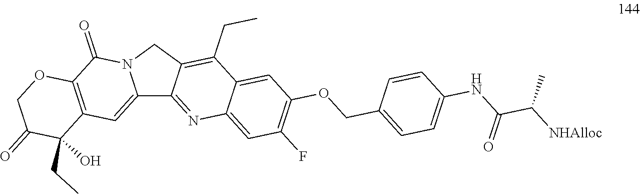

Compound 143 (1.05 g, 2.5 mmol) and compound 129 (0.70 g, 2.5 mmol) were dissolved in 50 mL of THF, cooled over an ice-water bath, and then PPh₃ (1.34 g, 5.0 mmol) and DEAD (0.89 g, 5.0 mmol) were added. After being stirred for 4 hours, the reaction was concentrated and purified on a silica gel column (5% MeOH in DCM) to give compound 144 (0.92 g, 55% yield).

### Example 99. Synthesis of (S)-2-amino-N-(4-((((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)methyl)phenyl) propanamide (145)

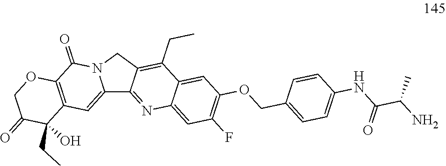 [SMILES: CCc1c2c(nc3cc(F)c(OCc4ccc(NC(=O)[C@H](C)N)cc4)cc13)-c1cc3c(c(=O)n1C2)OCC(=O)[C@]3(O)CC]

To a stirred solution of 144 (2.06 g, 3.069 mmol, 1.00 eq) in DCM (120 mL) at 0° C. were added Pd (PPh₃)₄(0.35 g, 0.307 mmol, 0.10 eq) and pyrrolidine (2.95 mL, 6.138 mmol, 2.00 eq). The resulting mixture was warmed to room temperature and stirred for 1.5 h before it was concentrated in vacuo. Flash column chromatography (silica gel, DCM: MeOH=10:1) afforded 145 (1.70 g, 79% yield) as a yellow solid.

### Example 100. Synthesis of allyl ((S)-1-(((S)-1-((4-((((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)methyl)phenyl)amino)-1-oxopropan-2-yl)amino)-3-methyl-1-oxobutan-2-yl) carbamate (146)

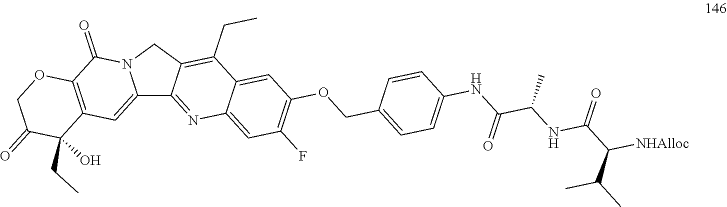

Compound 145 (1.35 g, 2.3 mmol) and ((allyloxy) carbonyl)-L-valine (0.68 g, 3.4 mmol) were dissolved in 50 mL of DMF, cooled over an ice-water bath, and HATU (1.31 g, 3.4 mmol) and diisopropylethylamine (569 μL, 3.4 mmol) were added, and stirred for 1 hour. The reaction mixture was concentrated, purified by a silica gel column chromatography, eluted with 10% MeOH in DCM to give compound 146 (1.38 mg, 78% yield).

### Example 101. Synthesis of (S)-2-amino-N—((S)-1-((4-((((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)methyl)phenyl)amino)-1-oxopropan-2-yl)-3-methylbutanamide (147)

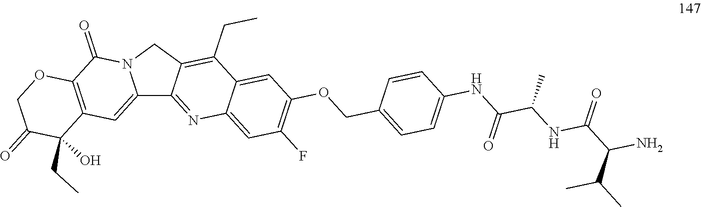 [SMILES: CCc1c2c(nc3cc(F)c(OCc4ccc(NC(=O)[C@H](C)NC(=O)[C@@H](N)C(C)C)cc4)cc13)-c1cc3c(c(=O)n1C2)OCC(=O)[C@]3(O)CC]

To a stirred solution of 146 (2.36 g, 3.069 mmol, 1.00 eq) in DCM (120 mL) at 0° C. were added Pd (PPh₃)₄(0.35 g, 0.307 mmol, 0.10 eq) and pyrrolidine (2.95 mL, 6.138 mmol, 2.00 eq). The resulting mixture was warmed to room temperature and stirred for 1.5 h before it was concentrated in vacuo. Flash column chromatography (silica gel, DCM: MeOH=10:1) afforded 147 (1.70 g, 79% yield) as a yellow solid.

### Example 102. Synthesis of tert-butyl (36S, 44S)-44-(((S)-1-(((S)-1-((4-((((S)-4, 11-diethyl-8-fluoro-4-hydroxy-3, 14-dioxo-3, 4, 12, 14-tetrahydro-IH-pyrano[3′, 4′:6, 7]indolizino[1, 2-b]quinolin-9-yl)oxy)methyl)phenyl)amino)-1-oxopropan-2-yl)amino)-3-methyl-1-oxobutan-2-yl) carbamoyl)-36-(4-(2, 5-dioxo-2, 5-dihydro-1H-pyrrol-1-yl) butanamido)-30, 37, 42-trioxo-2, 5, 8, 11, 14, 17, 20, 23, 25, 28-decaoxa-31, 38, 43-triazaheptatetracontan-47-oate (148)

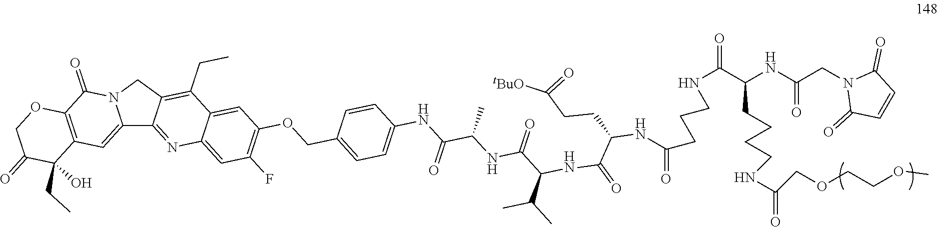

To a solution of compound 147 (494 mg, 0.72 mmol) and compound 16 (745 mg, 0.72 mmol) in DMF (15 mL) were added HATU (410 mg, 1.1 mmol) and DIEA (280 mg, 2.2 mmol) at 0° C. The mixture was stirred at 0° C. for 2 h. LC-MS showed the completion of the reaction. The mixture was then concentrated and purified by prep-HPLC to give compound 148 as a yellow solid (908 mg, 74.0% yield).

### Example 103. Synthesis of (36S, 44S)-44-(((S)-1-(((S)-1-((4-((((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)methyl)phenyl)amino)-1-oxopropan-2-yl)amino)-3-methyl-1-oxobutan-2-yl) carbamoyl)-36-(4-(2, 5-dioxo-2, 5-dihydro-IH-pyrrol-1-yl) butanamido)-30, 37, 42-trioxo-2, 5, 8, 11, 14, 17, 20, 23, 25, 28-decaoxa-31, 38, 43-triazaheptatetracontan-47-oic acid (149)

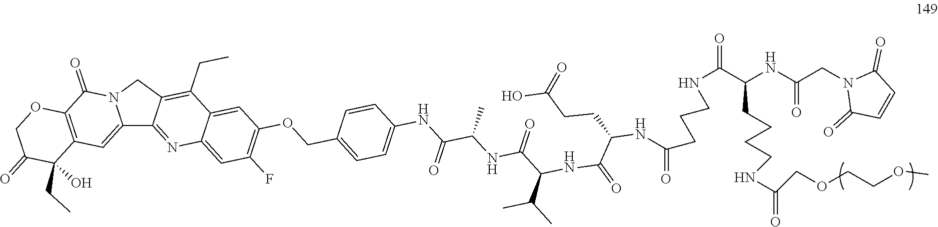

To a stirred solution of 148 (908 mg, 0.53 mmol) in DCM (20 mL) at room temperature was added TFA (20 mL), the resulting mixture was stirred at room temperature for 1 h, concentrated to afford 149 (900 mg, 100% yield) as a colorless oil.

### Example 104. Synthesis of compound (150)

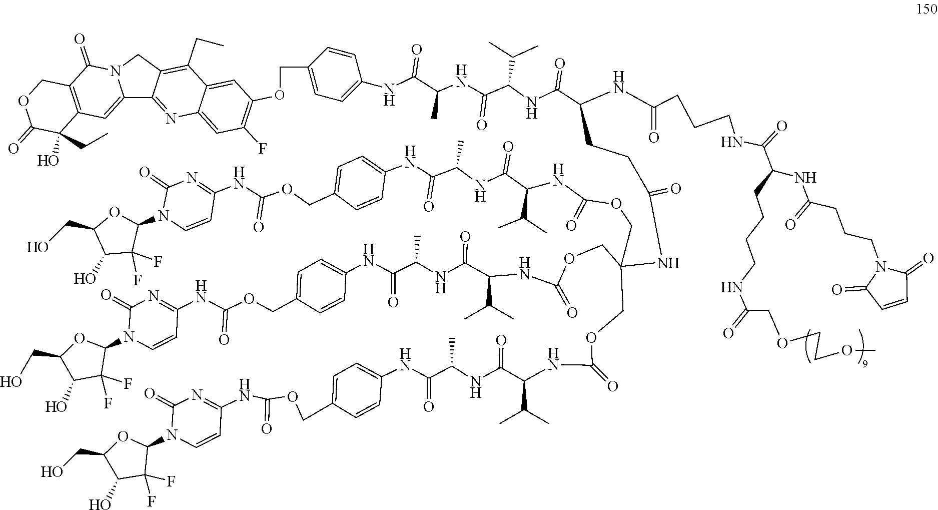

To a stirred solution of 149 (115.3 mg, 0.069 mmol, 1.50 eq) in DMF (2 mL) at room temperature were added HATU (26.4 mg, 0.069 mmol, 1.50 eq), 125 (90.0 mg, 0.046 mmol, 1.00 eq) and DIEA (25 μL, 0.139 mmol, 3.00 eq) in sequence. The resulting mixture was stirred at room temperature for 1 h.

Prep-HPLC purification and lyophilization afforded 150 (80.0 mg, 84% yield) as a white solid. ESI-MS m/z: calcd. for C₁₆₃H₂₁₃F₇N₂₉O₅₅ [M+H]⁺ 3589.4645; found 3589.4650.

### Example 105. Synthesis of (37S, 45S)-45-(3-(tert-butoxy)-3-oxopropyl)-37-(2-(2, 5-dioxo-2, 5-dihydro-1H-pyrrol-1-yl) acetamido)-31, 38, 43-trioxo-2, 5, 8, 11, 14, 17, 20, 23, 26, 29-decaoxa-32, 39, 44-triazahexatetracontan-46-oic acid (153)

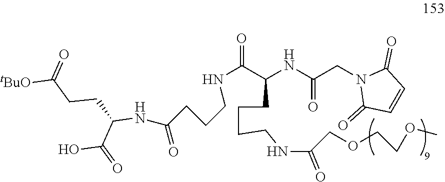

To a stirred solution of 152 (5.0 g, 5.78 mmol) in DCM (100 mL) at 0° C. were added NHS (0.73 g, 6.359 mmol) and EDC.HCl (2.22 g, 11.56 mmol). After 2 h L-glutamic acid 5-tert-butyl ester (1.52 g, 7.50m mmol) was added, and the resulting mixture was stirred at r.t. for 18 h before it was quenched with NH₄Cl. The layers were separated, and the aqueous layer was extracted with DCM. The combined organic layers were dried over anhydrous sodium sulfate and concentrated in vacuo. The crude product 153 (5.0 g, 95% yield) was used without further purification.

### Example 106. Synthesis of tert-butyl (37S, 45S)-37-(2-(2, 5-dioxo-2, 5-dihydro-1H-pyrrol-1-yl) acetamido)-45-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-31, 38, 43-trioxo-2, 5, 8, 11, 14, 17, 20, 23, 26, 29-decaoxa-32, 39, 44-triazaoctatetracontan-48-oate (154)

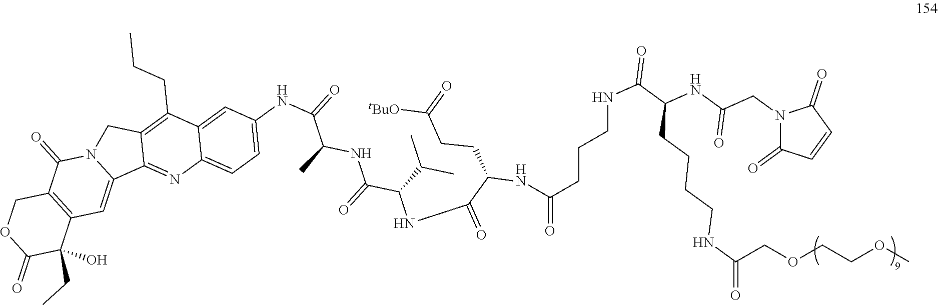

To a solution of compound 153 (494 mg, 0.72 mmol) and compound 138 (745 mg, 0.72 mmol) in DMF (15 mL) were added HATU (410 mg, 1.1 mmol) and DIEA (280 mg, 2.2 mmol) at 0° C. The mixture was stirred at 0° C. for 2 h. LC-MS showed the completion of the reaction. The mixture was purified by prep-HPLC to give compound 154 as a yellow solid (900 mg, 73.0% yield).

### Example 107. Synthesis of (37S, 45S)-37-(2-(2, 5-dioxo-2, 5-dihydro-1H-pyrrol-1-yl) acetamido)-45-(((S)-1-(((S)-1-(((S)-4-ethyl-4-hydroxy-3, 14-dioxo-11-propyl-3, 4, 12, 14-tetrahydro-IH-pyrano[3′, 4′:6, 7]indolizino[1, 2-b]quinolin-9-yl)amino)-1-oxopropan-2-yl)amino)-3-methyl-1-oxobutan-2-yl) carbamoyl)-31, 38, 43-trioxo-2, 5, 8, 11, 14, 17, 20, 23, 26, 29-decaoxa-32, 39, 44-triazaoctatetracontan-48-oic acid (155)

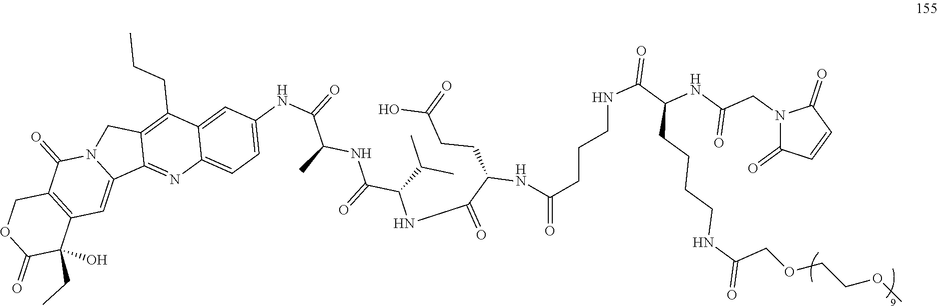

To a stirred solution of 154 (260.0 mg, 0.164 mmol) in DCM (10 mL) at room temperature was added TFA (40 mL). The resulting mixture was warmed to 40° C. and stirred for 4 h before it was concentrated in vacuo. The residue was purified by prep-HPLC to give 155 as a white solid (111.0 mg, 44% yield). ESI-MS m/z: calcd. for C₇₃H₁₀₇N₁₀O₂₅ [M+H]⁺ 1523.7403; found 1523.7404.

### Example 108. Synthesis of compound (156)

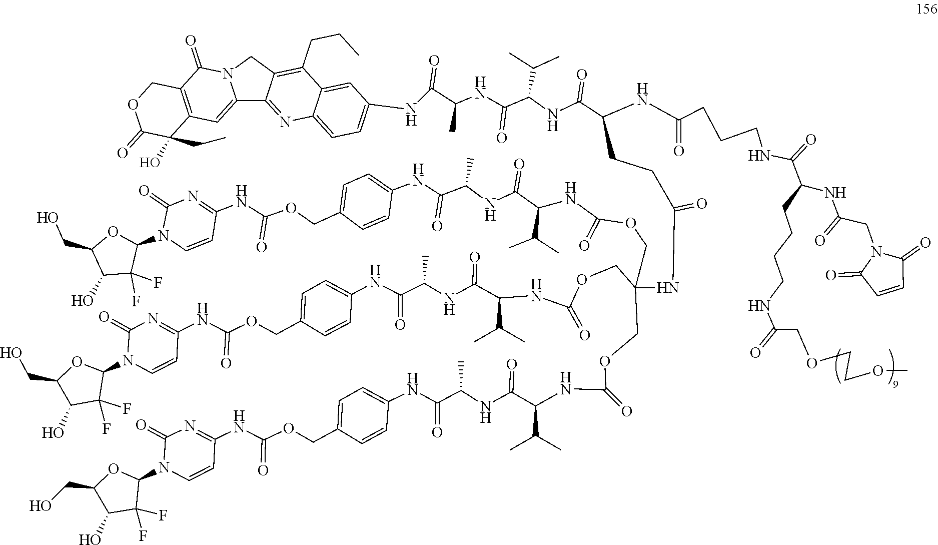

To a stirred solution of 155 (111.1 mg, 0.073 mmol, 1.80 eq) in DMF (2 mL) at room temperature were added HATU (27.7 mg, 0.073 mmol, 1.80 eq), 125 (78.9 mg, 0.041 mmol, 1.00 eq) and DIEA (25 mL, 0.146 mmol, 3.60 eq) in sequence. The resulting mixture was stirred at room temperature for 1 h.

Prep-HPLC purification and lyophilization afforded 156 (18.1 mg, 14% yield) as a white solid. ESI-MS m/z: calcd. for C₁₅₅H₂₀₆F₆N₂₉O₅₄ [M+H]⁺ 3451.4164; found 3451.4168.

### Example 109. Synthesis of 5-oxo-4, 9, 12-trioxa-6-azapentadec-1-en-15-oic acid (160)

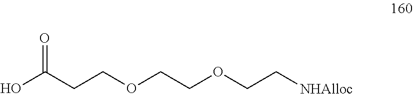

Compound 158 (5.00 g, 28 mmol) was dissolved in 100 mL of DMF, to which compound 159 (6.01 g, 30 mmol) and diisopropylethylamine (93 μL, 56 mmol) were added and stirred overnight. The reaction mixture was concentrated to afford compound 160 (7.37 g, 100% yield). ESI-MS m/z: [M+H]+calcd. for C₁₁H₁₉NO₆ 262.12; found 262.12.

### Example 110. Synthesis of allyl (2-(2-(3-((1, 3-dihydroxy-2-(hydroxymethyl) propan-2-yl)amino)-3-oxopropoxy) ethoxy)ethyl) carbamate (161)

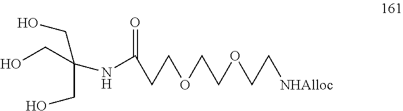

Compound 160 (7.37 g, 28 mmol) was dissolved in 200 mL of DCM, and then NHS (3.5717 g, 31 mmol) and DCC (6.9886 g, 34 mmol) were added, and stirred at room temperature for 4 hours. The white solid was filtered and the filtrate was concentrated. This crude product and compound 115 (3.14 g, 26 mmol) were dissolved in 30 mL of DMF, and diisopropylethylamine (4.3 mL, 26 mmol) was added. The reaction was stirred for 2.5 hours, and concentrated. The residue was purified by prep-HPLC to give compound 161 (1.80 g, 19% yield). ESI-MS m/z: [M+H]⁺ calcd. for C₁₅H₂₈N₂O₈ 365.18; found 365.18.

### Example 111. Synthesis of allyl (1-(4-nitrophenoxy)-4, 4-bis((((4-nitrophenoxy) carbonyl)oxy)methyl)-1, 6-dioxo-2, 9, 12-trioxa-5-azatetradecan-14-yl) carbamate (162)

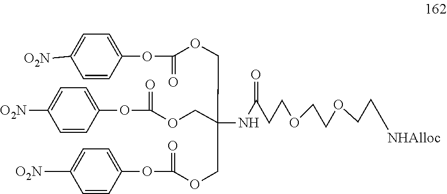

Compound 161 (1.60 g, 4.4 mmol) was dissolved in 100 mL of THF, cooled over an ice-water bath. After compound 112 (3.98 g, 19.8 mmol) was added, pyridine (2.08 g, 26.3 mmol) was added dropwise, resulting in the precipitation of a large amount of white solid. The ice bath was removed and the reaction was stirred at room temperature for 3.5 hours, then concentrated, and purified by a silica gel column (8% ethyl acetate in petroleum ether) to give compound 162 (1.47 g, 39% yield). ESI-MS m/z: [M +H]⁺calcd. for C₃₆H₃₇N₅O₂₀ 860.20; found 860.20.

### Example 112. Synthesis of di-tert-butyl (2S, 12S)-7-(((((S)-1-(tert-butoxy)-3-methyl-1-oxobutan-2-yl) carbamoyl)oxy)methyl)-2, 12-diisopropyl-4, 10-dioxo-7-(5-oxo-4, 9, 12-trioxa-6-azapentadec-1-en-15-amido)-5, 9-dioxa-3, 11-diazatridecanedioate (163)

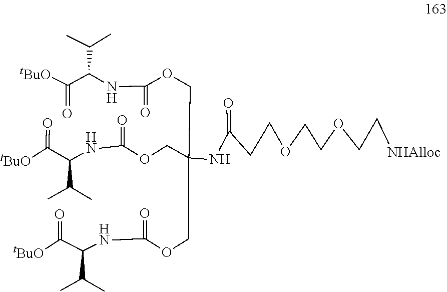

Compound 162 (1.47 g, 1.7 mmol) was dissolved in 20 mL of THF, and then compound 120 (1.78 g, 10.3 mmol) and diisopropylethylamine (22 μL, 13.7 mmol) were added and stirred for 3 hours. After concentration, a silica gel column purification (70% ethyl acetate in petroleum ether) compound 163 was obtained (0.45 g, 27% yield). ESI-MS m/z: [M+H]⁺calcd. for C₄₅H₇₉N₅O₁₇ 962.55; found 962.55.

### Example 113. Synthesis of (2S, 12S)-7-(((((S)-1-carboxy-2-methylpropyl) carbamoyl)oxy)methyl)-2, 12-diisopropyl-4, 10-dioxo-7-(5-oxo-4, 9, 12-trioxa-6-azapentadec-1-en-15-amido)-5, 9-dioxa-3, 11-diazatridecanedioic acid (164)

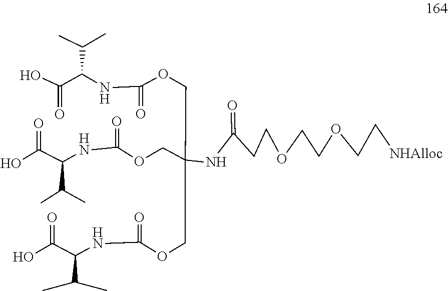

Compound 163 (0.45 g, 0.47 mmol) was dissolved in 5 mL of DCM and stirred with 10 mL of formic acid at room temperature for 2.5 hours. Concentration of the reaction mixture gave a crude compound 164 (0.37 g, 100% yield). ESI-MS m/z: [M+H]⁺ calcd. for C₃₃H₅₅N₅O₁₇ 794.36; found 794.36.

### Example 114. Synthesis of compound (165)

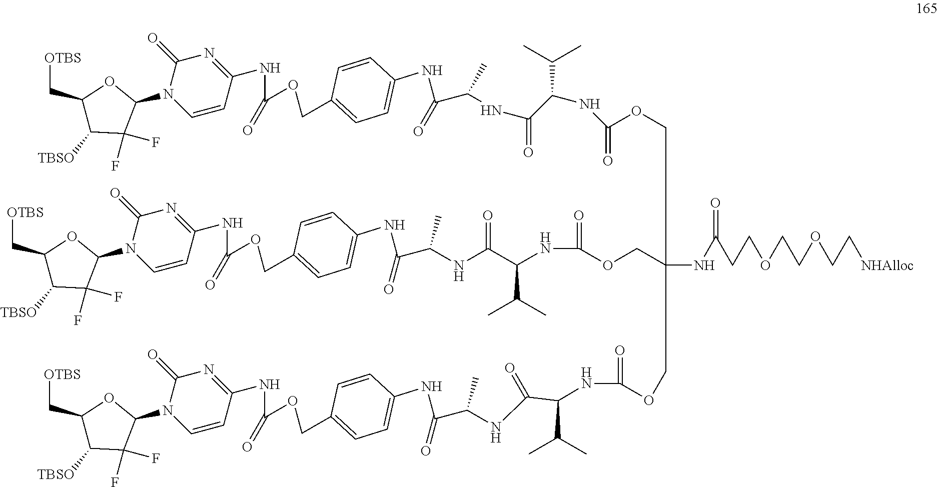

Compound 164 (0.37 g, 0.47 mmol) was dissolved in 10 mL of DMF, and HATU (0.5510 g, 1.45 mmol) and diisopropylethylamine (231 μL, 1.39 mmol) were added. The mixture was stirred at room temperature for 5 min., and then compound 114 (1.1000 g, 1.54 mmol) and diisopropylethylamine (231 μL, 1.39 mmol) were added. After stirring for 5 hours, the reaction was concentrated, and purified by a silica gel column (5-15% MeOH in DCM) to give compound 165 (1.34 g, 100% yield).

### Example 115. Synthesis of compound (166)

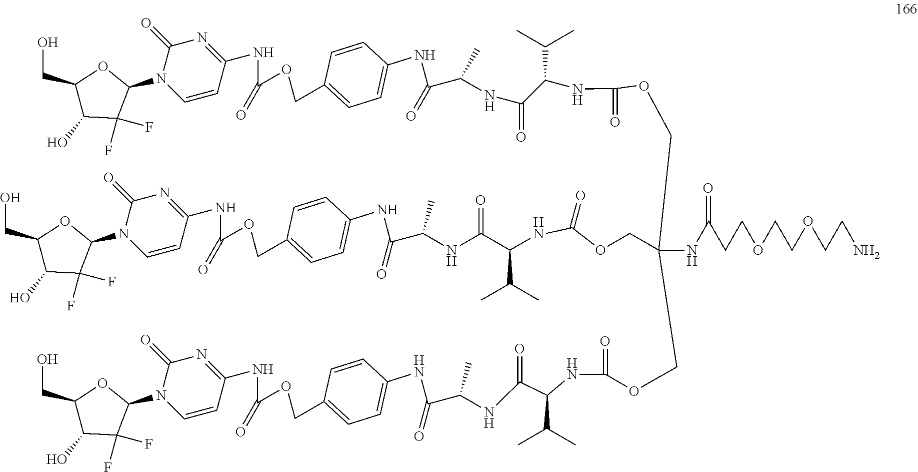 [SMILES: CC(C)[C@H](NC(=O)OCC(COC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)(COC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)NC(=O)CCOCCOCCN)C(=O)N[C@@H](C)C(=O)Nc1ccc(COC(=O)Nc2ccn([C@@H]3O[C@H](CO)[C@@H](O)C3(F)F)c(=O)n2)cc1]

Compound 165 (1.24 g, 0.43 mmol) was dissolved in 20 mL of DCM, cooled over an ice-water bath, and Pd(PPh₃)₄(0.10 g, 0.09 mmol) was added. Tetrachloropyrrole (pyrrolidine) (0.15 g, 2.16 mmol) was added dropwise, and the ice bath was removed, the mixture was stirred at room temperature for 3 hours. After concentration, the residue was dissolved in 20 mL of THF, and 4 mL of tetrabutylammonium fluoride was added. The reaction was stirred for 0.5 hours, and then quenched by a small amount of methanol, then concentrated and purified by prep-HPLC to give compound 166 (164 mg, 17% yield). ESI-MS m/z: [M+H]⁺ calcd. for C₈₉H₁₁₄F₆N₂₀O₃₃ 2105.78; found 2105.78.

### Example 116. Synthesis of compound (167)

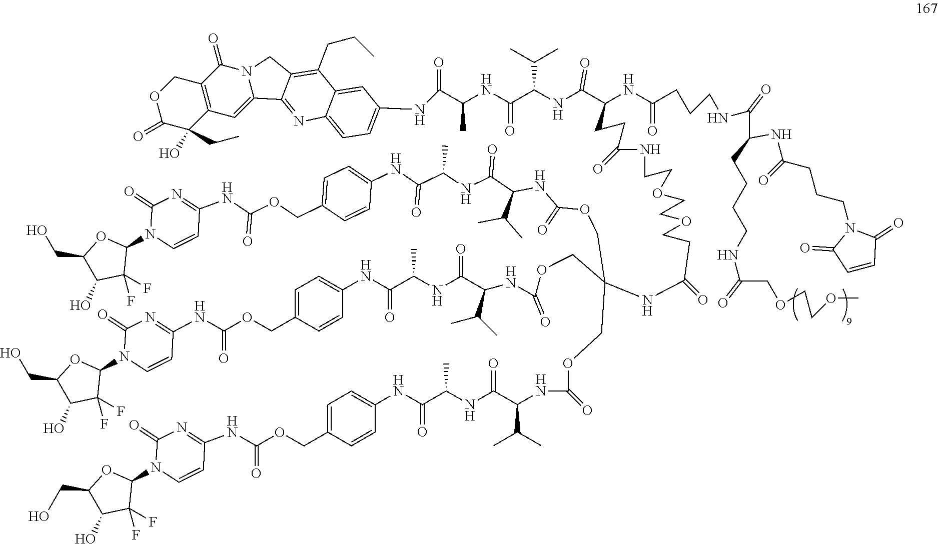

Compound 140 (0.0884 g, 0.057 mmol) was dissolved in 2 mL of DMF, and HATU (0.0221 g, 0.057 mmol) was added. After stirring for 10 min., compound 166 (0.0800 g, 0.038 mmol) was added, followed by diisopropylethylamine (19 μL. 0.114 mmol) dropwise. The reaction was stirred for 1 hour, and purified by prep-HPLC to give compound 167 (30 mg, 210% yield). ESI-MS m/z: [M+2H]²⁺ calcd. for C₁₆₄H₂₂₂F₆N₃₀O₅₇ 1819.76; found 1819.76.

### Example 117. Synthesis of compound (169)

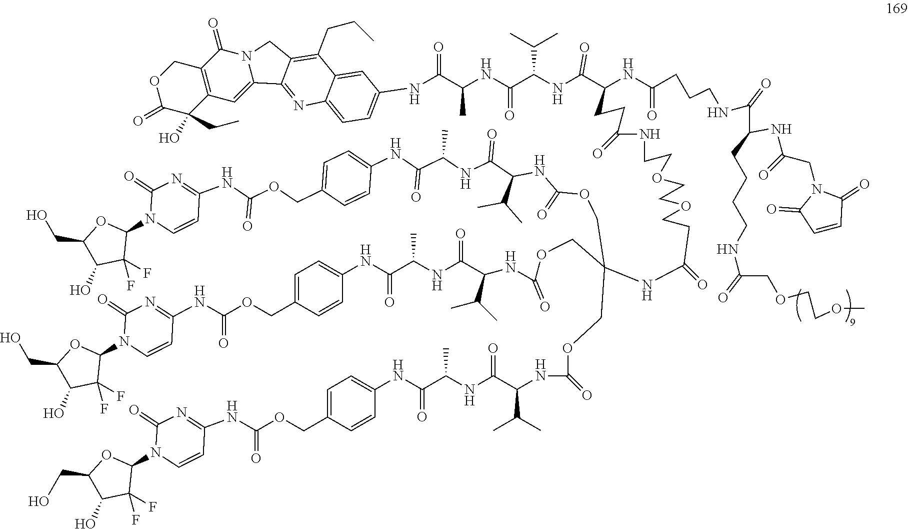

Compound 155 (0.0671 g, 0.044 mmol) was dissolved in 2 mL of DMF, and HATU (0.0192 g, 0.048 mm mol) was added, and stirred for 10 min. Compound 166 (0.0840 g, 0.04 mmol) was added, followed by diisopropylethylamine (17 μL, 0.10 mmol) dropwise. The reaction was stirred for 1.5 hours, and purified by prep-HPLC to give compound 169 (20 mg, 14% yield). ESI-MS m/z: [M+2H]2+calcd. for C₁₆₂H₂₁₈F₆N₃₀O₅₇ 1805.75; found 1805.75.

### Example 118. Synthesis of tert-butyl ((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)amino)-2-oxoethyl)amino)-1-oxopropan-2-yl)amino)-3-methyl-1-oxobutan-2-yl) carbamate (172)

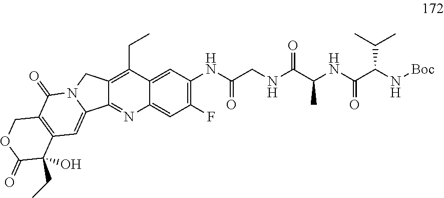 [SMILES: CCc1c2c(nc3cc(F)c(NC(=O)CNC(=O)[C@H](C)NC(=O)[C@@H](NC(=O)OC(C)(C)C)C(C)C)cc13)-c1cc3c(c(=O)n1C2)COC(=O)[C@]3(O)CC]

Compound 171 (1.07 g, 2.3 mmol) and compound 129 (0.99 g, 3.4 mmol) were dissolved in 50 mL of DCM, cooled over an ice-water bath, and HATU (1.31 g, 3.4 mmol), HOAt (0.31 g, 2.3 mmol) and diisopropylethylamine (569 μL. 3.4 mmol) were added. After being stirred for 1 hour, the reaction was concentrated, purified on a silica gel column (6% MeOH in DCM) to give compound 172 (956 mg, 57% yield). ESI-MS m/z: [M+H]⁺calcd. for C₃₇H₄₅FN₆O₉ 737.80; found 737.80.

### Example 119. Synthesis of (S)-2-amino-N—((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)amino)-2-oxoethyl)amino)-1-oxopropan-2-yl)-3-methylbutanamide (173)

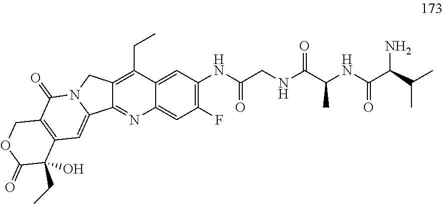 [SMILES: CCc1c2c(nc3cc(F)c(NC(=O)CNC(=O)[C@H](C)NC(=O)[C@@H](N)C(C)C)cc13)-c1cc3c(c(=O)n1C2)COC(=O)[C@]3(O)CC]

Compound 172 (0.8900 g, 1.2 mmol) was dissolved in 30 mL of DCM, and stirred with 5 mL of TFA for 1 hour, and then concentrated to give 173 (0.77 g, 100% yield). ESI-MS m/z: [M+H]⁺ calcd. for C₃₂H₃₇FN₆O₇ 637.68; found 637.68.

### Example 120. Synthesis of tert-butyl (37S, 45S)-45-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)amino)-2-oxoethyl)amino)-1-oxopropan-2-yl)amino)-3-methyl-1-oxobutan-2-yl) carbamoyl)-37-(4-(2, 5-dioxo-2, 5-dihydro-IH-pyrrol-1-yl) butanamido)-31, 38, 43-trioxo-2, 5, 8, 11, 14, 17, 20, 23, 26, 29-decaoxa-32, 39, 44-triazaoctatetracontan-48-oate (174)

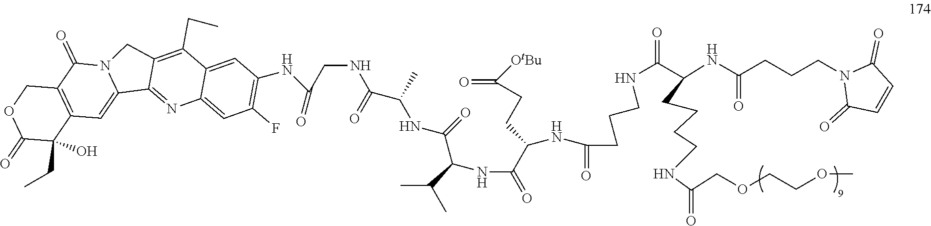

Compound 173 (0.7600 g, 1.2 mmol) was dissolved in 15 mL of DMF, to which diisopropylethylamine (304 μL,1.8 mmol), compound 127 (0.9650 g, 0.9 mmol) and HATU (0.4561 g, 1.2 mmol) were added. After being stirred for 0.5 hours, the reaction was diluted with 200 mL of DCM, washed with 100 mL of water, and 100 mL of brine. The organic phase was dried over anhydrous sodium sulfate and concentrated to give compound 174 (1.53 g, 100% yield). ESI-MS m/z: [M+H]⁺ calcd. for C₈₀H₁₁₈FN₁₁O₂₆ 1669.87; found 1669.87.

### Example 121. Synthesis of (37S, 45S)-45-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)amino)-2-oxoethyl)amino)-1-oxopropan-2-yl)amino)-3-methyl-1-oxobutan-2-yl) carbamoyl)-37-(4-(2, 5-dioxo-2, 5-dihydro-1H-pyrrol-1-yl) butanamido)-31, 38, 43-trioxo-2, 5, 8, 11, 14, 17, 20, 23, 26, 29-decaoxa-32, 39, 44-triazaoctatetracontan-48-oic acid (175)

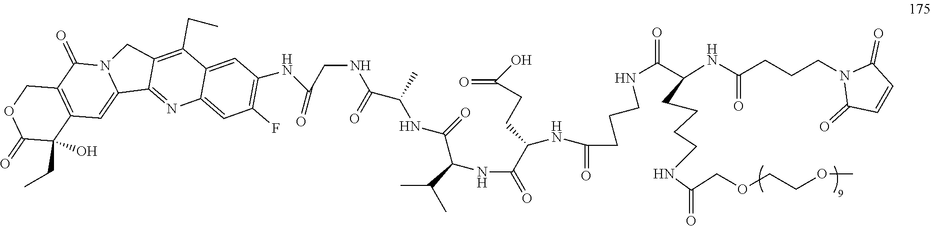

Compound 174 (1.5300 g, 0.9 mmol) was dissolved in 20 mL of DCM, and stirred with 20 mL of TFA for 1.5 hours. The reaction was concentrated and purified by prep-HPLC to give compound 175 (0.9 g, 61% yield). ESI-MS m/z: [M+H]⁺calcd. for C₇₆H₁₁₀FN₁₁₀O₂₆ 1613.77; found 1613.77.

### Example 122. Synthesis of compound (176)

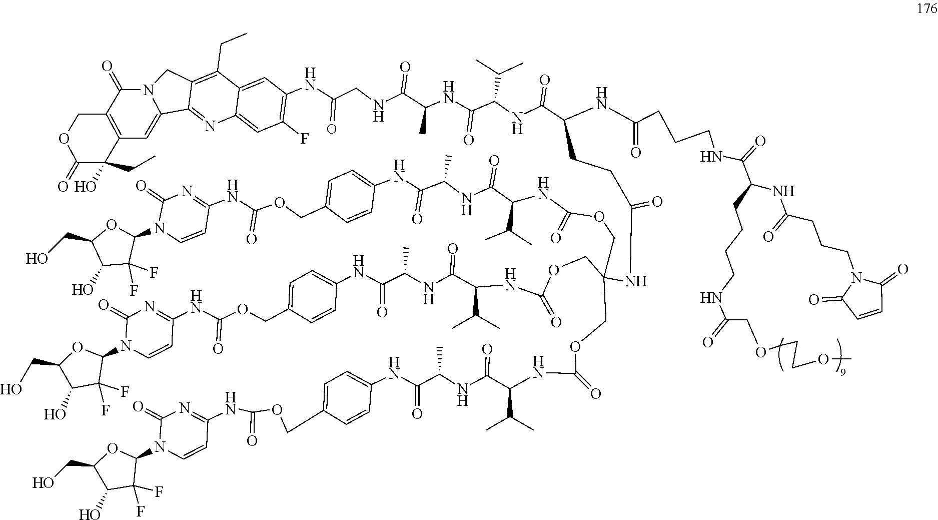

To a solution of compound 175 (0.1860 g, 0.12 mmol) dissolved in 2 mL of DMF, HATU (0.0441 g, 0.12 mmol) was added, and stirred for 10 min. Compound 125 (0.1500 g, 0.08 mmol) and diisopropylethylamine (38 μL, 0.23 mmol) were added and stirred for 2 hours. Prep-HPLC purification afforded compound 176 (108 mg, 40% yield). ESI-MS m/z: [M+2H]²⁺calcd. for C₁₅₈H₂₀₉F₇N₃₀O₅₅ 1771.77; found 1771.77.

### Example 123. Synthesis of tert-butyl (S)-(1-((4-(bromomethyl)phenyl)amino)-1-oxopropan-2-yl) carbamate (179)

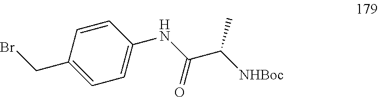 [SMILES: C[C@H](NC(=O)OC(C)(C)C)C(=O)Nc1ccc(CBr)cc1]

Compound 178 (2.94 g, 10.0 mmol) was dissolved in 100 mL of THF, and PPh₃ (3.14 g, 12.0 mmol) and NBS (2.14 g, 12.0 mmol) were added, and stirred for 4 hours. Flash column chromatography (silica gel, petroleum ether: ethyl acetate=3:1) afforded 179 (2.32 g, 65% yield) as a yellow solid.

### Example 124. Synthesis of (S)-4-(((9H-fluoren-9-yl) methoxy) carbonyl)-1-(4-(2-((tert-butoxycarbonyl)amino) propanamido)benzyl)-1-methylpiperazin-1-ium (181)

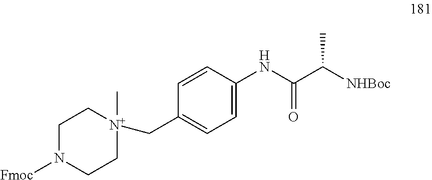

A solution of 179 (3.60 g, 10.00 mmol) and 180 (3.22 g, 10.00 mmol) in MeCN (60 mL) was stirred at room temperature for 1 h and concentrated in vacuo. The residue was triturated with ethyl acetate and filtered to afford 181 (4.55 g, 75% yield). ESI-MS m/z: calcd. for C₃₅H₄₃N₄O₅ [M]⁺ 599.32; found 599.32.

### Example 125. Synthesis of (S)-1-(4-(2-((tert-butoxycarbonyl)amino) propanamido)benzyl)-1-methylpiperazin-1-ium (182)

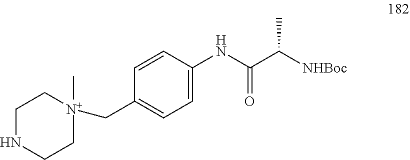 [SMILES: C[C@H](NC(=O)OC(C)(C)C)C(=O)Nc1ccc(C[N+]2(C)CCNCC2)cc1]

To a stirred solution of 181 (2.5 g, 4.17 mmol) in DMF (25 mL), piperidine (5 mL) was added at room temperature and stirred for further 15 min. before it was concentrated in vacuo. The residue was triturated with DCM: ethyl acetate=1:1 and filtered to afford 182 (1.46 g, 93% yield). ESI-MS m/z: calcd. for C₂₀H₃₃N₄O₃ [M]⁺ 377.25; found 377.25.

### Example 126. Synthesis of 1-(4-((S)-2-((tert-butoxycarbonyl)amino) propanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (184)

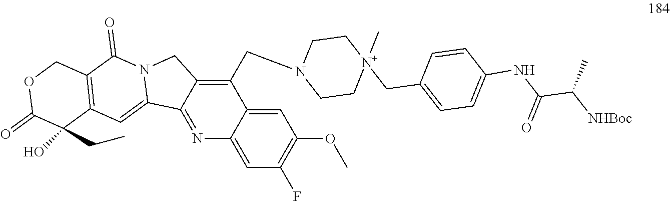 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)OC(C)(C)C)cc2)CC1]

To a solution of 182 (1.28 g, 3.39 mmol), 183 (1.51 g, 3.39 mmol) in NMP (25 mL) 4-methylmorpholine (0.34 g, 3.39 mmol) was added at room temperature. The resulting mixture was stirred at room temperature for further 1 h before it was concentrated in vacuo. The residue was triturated with ethyl acetate and filtered to afford 184 (2.20 g, 83% yield).

### Example 127. Synthesis of 1-(4-((S)-2-aminopropanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (185)

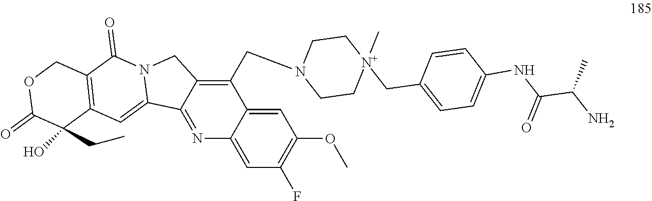 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)N)cc2)CC1]

To a stirred solution of 184 (2.00 g, 2.55 mmol, 1.00eq) in DCM (27 mL) at room temperature was added TFA (9 mL). The resulting mixture was stirred at room temperature for 40 min., concentrated in vacuo. The residue was triturated with ethyl acetate (40 mL) and filtered to afford 185 (2.10 g, 120% yield) as a brown solid. ESI-MS m/z: calcd. for C₃₈H₆₆N₄O₁₄ [M+H]⁺ 686.3144; found 686.3144.

### Example 128. Synthesis of 1-(4-((S)-2-((S)-2-((tert-butoxycarbonyl)amino)-3-methylbutanamido) propanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (186)

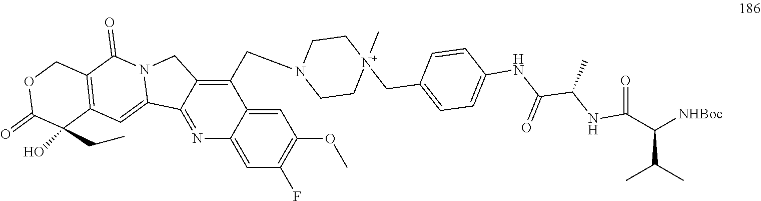 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)[C@@H](NC(=O)OC(C)(C)C)C(C)C)cc2)CC1]

To a stirred solution of 185 (1.75 g, 2.55 mmol, 1.00 eq) in DMF (20 mL) at room temperature was added HATU (1.02 g, 2.68 mmol, 1.05eq). After stirring for 10 min., 55 (0.58 g, 2.68 mmol, 1.05eq) and DIEA (0.69 g, 5.36 mmol, 2.10 eq) were added. The resulting mixture was stirred at room temperature for 1 h, and concentrated in vacuo. Flash column chromatography (silica gel, DCM: MeOH=7:3) afforded 186 (1.71 g, 75% yield) as a brown solid. ESI-MS m/z: calcd. for C₃₈H₆₆N₄O₁₄ [M+H]⁺ 887.4509 found 887.4509.

### Example 129. Synthesis of 1-(4-((S)-2-((S)-2-amino-3-methylbutanamido) propanamido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (187)

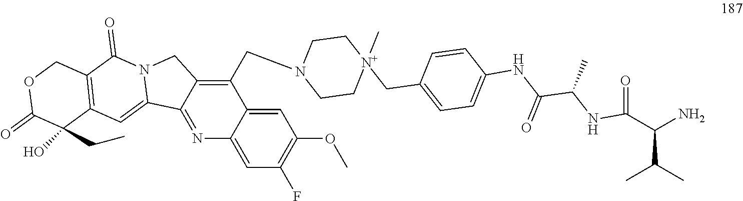 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)[C@@H](N)C(C)C)cc2)CC1]

To a stirred solution of 186 (2.35 g, 2.66 mmol, 1.00eq) in DCM (24 mL) at room temperature was added TFA (8 mL). The resulting mixture was stirred at room temperature for 35 min., concentrated in vacuo. The residue was triturated with ethyl acetate (40 mL) to afford 187 (1.24 g, 59% yield) as a brown solid. ESI-MS m/z: calcd. for C₃₈H₆₆N₄O₁₄ [M+H]⁺ 785.3829; found 785.3829.

### Example 130. Synthesis of 1-(4-((37S, 45S, 48S, 51S)-45-(3-(tert-butoxy)-3-oxopropyl)-37-(4-(2, 5-dioxo-2, 5-dihydro-1H-pyrrol-1-yl) butanamido)-48-isopropyl-51-methyl-31, 38, 43, 46, 49-pentaoxo-2, 5, 8, 11, 14, 17, 20, 23, 26, 29-decaoxa-32, 39, 44, 47, 50-pentaazadopentacontan-52-amido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (188)

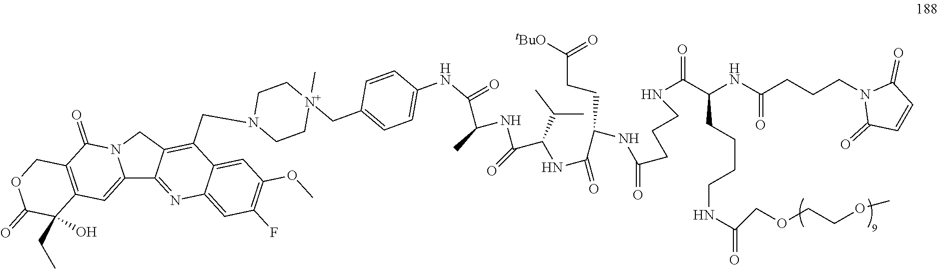

To a stirred solution of 127 (1.53 g, 1.46 mmol, 1.10 eq) in DMF (10 mL) at room temperature was added HATU (0.55 g, 1.46 mmol, 1.10 eq). After stirring for 10 min., 187 (1.04 g, 1.33 mmol, 1.00 eq) and DIEA (0.38 g, 2.92 mmol, 2.20 eq) were added and stirred at room temperature for 1 h, concentrated in vacuo. Flash column chromatography (silica gel, DCM: MeOH=7:3) afforded 188 (1.44 g, 59% yield) as a brown solid. ESI-MS m/z: calcd. for C₃₈H₆₆N₄O₁₄ [M+H]⁺ 1816.9354 found 1816.9354.

### Example 131. Synthesis of 1-(4-((37S, 45S, 48S, 51S)-45-(2-carboxyethyl)-37-(4-(2, 5-dioxo-2, 5-dihydro-1H-pyrrol-1-yl) butanamido)-48-isopropyl-51-methyl-31, 38, 43, 46, 49-pentaoxo-2, 5, 8, 11, 14, 17, 20, 23, 26, 29-decaoxa-32, 39, 44, 47, 50-pentaazadopentacontan-52-amido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (189)

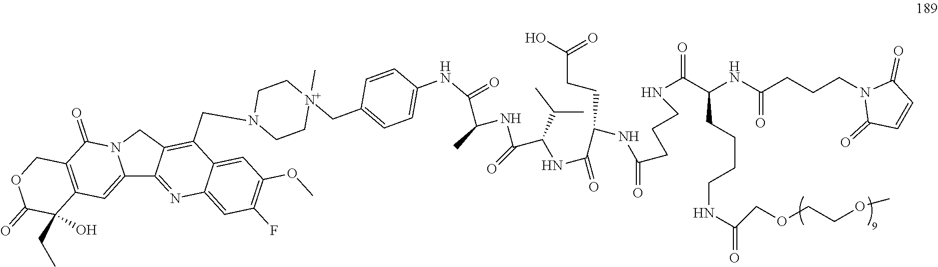

To a stirred solution of 188 (1.20 g, 0.66 mmol, 1.00e) in DCM (15 mL) at room temperature was added TFA (5 mL). The resulting mixture was stirred at room temperature for 1 h, concentrated in vacuo.

Prep-HPLC purification afforded 189 (0.65 g, 56% yield) as a white solid. ESI-MS m/z: calcd. for C₃₈H₆₆N₄O₁₄ [M+H]⁺ 1760.8728 found 1760.8728.

### Example 132. Synthesis of compound (190)

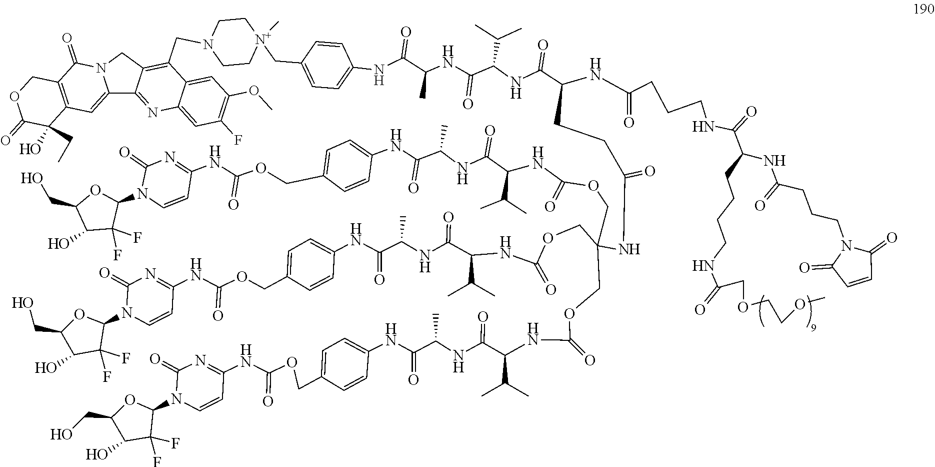

To a stirred solution of 189 (176.4 mg, 0.100 mmol, 1.30eq) in DMF (4 mL) at room temperature was added HATU (38.1 mg, 0.100 mmol, 1.30eq). After stirring for 10 min., 124 (150.0 mg, 0.077 mmol, 1.00 eq) and DIEA (25.9 mg, 0.200 mmol, 2.60 eq) were added. The resulting mixture was stirred at room temperature for 1 h, concentrated in vacuo. Prep-HPLC purification afforded 190 (7.0 mg, 2.5% yield) as a white solid. ESI-MS m/z: calcd. for C₁₅₅H₂₀₆F₆N₂₉O₅₄ [M+2H]²⁺ 1844.7745; found 1844.7745.

### Example 133. Synthesis of ((2R, 3R, 5R)-5-(4-amino-2-oxopyrimidin-1 (2H)-yl)-3-((tert-butoxycarbonyl)oxy)-4, 4-difluorotetrahydrofuran-2-yl)methyl tert-butyl carbonate (192)

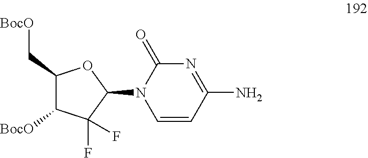

To a stirred solution of gemcitabine (5.00 g, 18.997 mmol, 1.00 eq) in NaOH (1.0 M in water, 500 mL) at room temperature was added a solution of Boc₂O (62.19 g, 284.954 mmol, 15.00 eq) in 1, 4-dioxane (400 mL). The resulting mixture was stirred at room temperature for 4 h before it was quenched with water (200 mL). The layers were separated, and the aqueous layer was extracted with ethyl acetate (3×300 mL). The combined organic layers were dried over anhydrous sodium sulfate and concentrated in vacuo. Flash column chromatography (DCM: MeOH=20:1) afforded 192 (3.00 g, 38% yield) as white solid. ESI-MS m/z: calcd. for C₁₉H₂₈F₂N₃₀₈ [M+H]⁺ 464.1839; found 464.1944.

### Example 134. Synthesis of tert-butyl (2-((2-((2-((1-((2R, 4R, 5R)-4-((tert-butoxycarbonyl)oxy)-5-(((tert-butoxycarbonyl)oxy)methyl)-3, 3-difluorotetrahydrofuran-2-yl)-2-oxo-1, 2-dihydropyrimidin-4-yl)amino)-2-oxoethyl)amino)-2-oxoethyl)amino)-2-oxoethyl) carbamate (194)

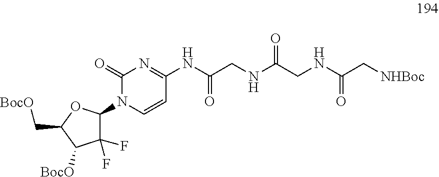

To a stirred solution of 192 (250 mg, 0.539 mmol, 1.00 eq) and 193 (156 mg, 0.539 mmol, 1.00 eq) in acetonitrile (5 mL) at 0° C. were added NMI (88 mg, 1.078 mmol, 2.00 eq) and TCFH (151 mg, 0.539 mmol, 1.00 eq). The resulting mixture was stirred at room temperature for 1 h and concentrated in vacuo. Flash column chromatography (DCM: MeOH=95:5) afforded 194 (394 mg, 100% yield) as a white solid.

### Example 135. Synthesis of 2-amino-N-(2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl) acetamide (195)

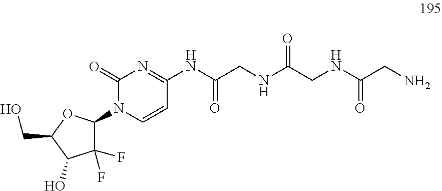 [SMILES: NCC(=O)NCC(=O)NCC(=O)Nc1ccn([C@@H]2O[C@H](CO)[C@@H](O)C2(F)F)c(=O)n1]

To a stirred solution of 194 (394 mg, 0.539 mmol, 1.00eq) in DCM (8 mL) at room temperature was added TFA (8 mL). The resulting mixture was stirred at room temperature for 1 h and concentrated in vacuo. The residue was triturated with ethyl acetate (20 mL), filtered to afford 195 (303 mg, 100% yield).

### Example 136. Synthesis of 3, 3′-((2-amino-2-((2-carboxyethoxy)methyl) propane-1, 3-diyl)bis (oxy))dipropionic acid (197)

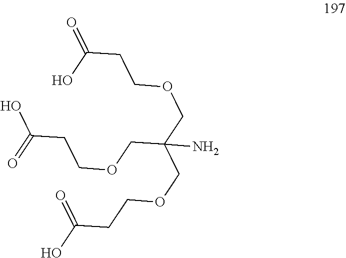 [SMILES: NC(COCCC(=O)O)(COCCC(=O)O)COCCC(=O)O]

To a stirred solution of 196 (505 mg, 1.00 mmol, 1.00e) in DCM (8 mL) at room temperature was added TFA (8 mL). The resulting mixture was stirred at room temperature for 3 h, concentrated in vacuo. Prep-HPLC purification afforded 197 (283 mg, 84% yield) as a white solid.

### Example 137. Synthesis of 11, 11-bis((2-carboxyethoxy)methyl)-2, 2-dimethyl-4, 9-dioxo-3, 13-dioxa-5, 10-diazahexadecan-16-oic acid (199)

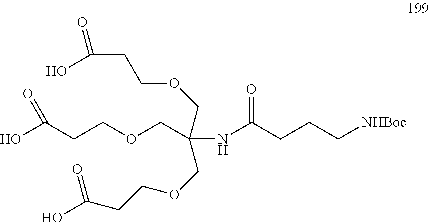 [SMILES: CC(C)(C)OC(=O)NCCCC(=O)NC(COCCC(=O)O)(COCCC(=O)O)COCCC(=O)O]

To a stirred solution of 197 (283 mg, 0.84 mmol) in DMF (5 mL) at room temperature were added 198 (252 mg, 0.84 mmol) and DIEA (254 mg, 2.00 mmol). The mixture was stirred at room temperature for 2 h and purified by prep-HPLC, lyophilized to afford 199 (263 mg, 60% yield) as a white solid.

### Example 138. Synthesis of tert-butyl (1-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-15, 15-bis (14-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-5, 8, 11, 14-tetraoxo-2-oxa-6, 9, 12-triazatetradecyl)-1, 4, 7, 10, 17-pentaoxo-13-oxa-3, 6, 9, 16-tetraazaicosan-20-yl) carbamate (200)

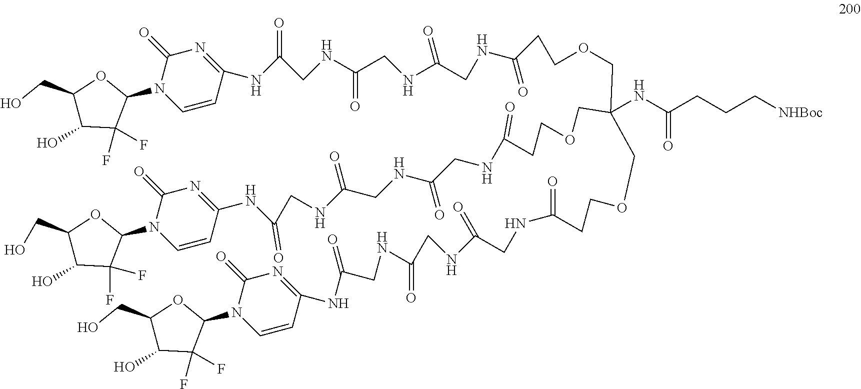 [SMILES: CC(C)(C)OC(=O)NCCCC(=O)NC(COCCC(=O)NCC(=O)NCC(=O)NCC(=O)Nc1ccn([C@@H]2O[C@H](CO)[C@@H](O)C2(F)F)c(=O)n1)(COCCC(=O)NCC(=O)NCC(=O)NCC(=O)Nc1ccn([C@@H]2O[C@H](CO)[C@@H](O)C2(F)F)c(=O)n1)COCCC(=O)NCC(=O)NCC(=O)NCC(=O)Nc1ccn([C@@H]2O[C@H](CO)[C@@H](O)C2(F)F)c(=O)n1]

To a stirred solution of 199 (261 mg, 0.50 mmol) in DMF (10 mL) at room temperature were added 195 (651 mg, 1.50 mmol), HATU (697.0 mg, 1.83 mmol) and DIEA (355.0 mg, 2.74 mmol). The reaction mixture was stirred at room temperature for 0.5 h. The reaction mixture was diluted with DCM (150 mL), washed with water (100 mL), and concentrated. The residue was purified by a silica gel column, eluted with DCM and methanol, to afford 200 (646 mg, 73% yield) as a white solid.

### Example 139. Synthesis of 3, 3′-((2-(4-aminobutanamido)-2-(14-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-5, 8, 11, 14-tetraoxo-2-oxa-6, 9, 12-triazatetradecyl) propane-1, 3-diyl)bis (oxy))bis(N-(2-((2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)amino)-2-oxoethyl) propanamide) (201)

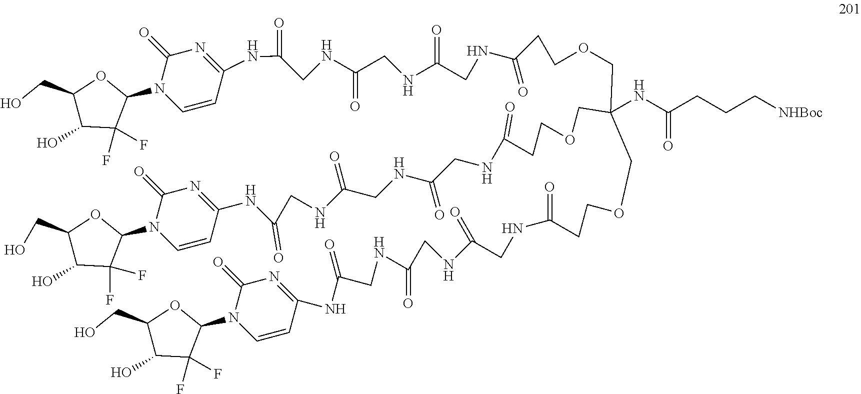 [SMILES: CC(C)(C)OC(=O)NCCCC(=O)NC(COCCC(=O)NCC(=O)NCC(=O)NCC(=O)Nc1ccn([C@@H]2O[C@H](CO)[C@@H](O)C2(F)F)c(=O)n1)(COCCC(=O)NCC(=O)NCC(=O)NCC(=O)Nc1ccn([C@@H]2O[C@H](CO)[C@@H](O)C2(F)F)c(=O)n1)COCCC(=O)NCC(=O)NCC(=O)NCC(=O)Nc1ccn([C@@H]2O[C@H](CO)[C@@H](O)C2(F)F)c(=O)n1]

To a stirred solution of 200 (646 mg, 0.38 mmol) in DCM (8 mL) at room temperature was added TFA (8 mL). The resulting mixture was stirred at room temperature for 3 h, concentrated in vacuo. Prep-HPLC purification afforded 201 (508 mg, 80% yield) as a white solid.

### Example 140. Synthesis of compound 202

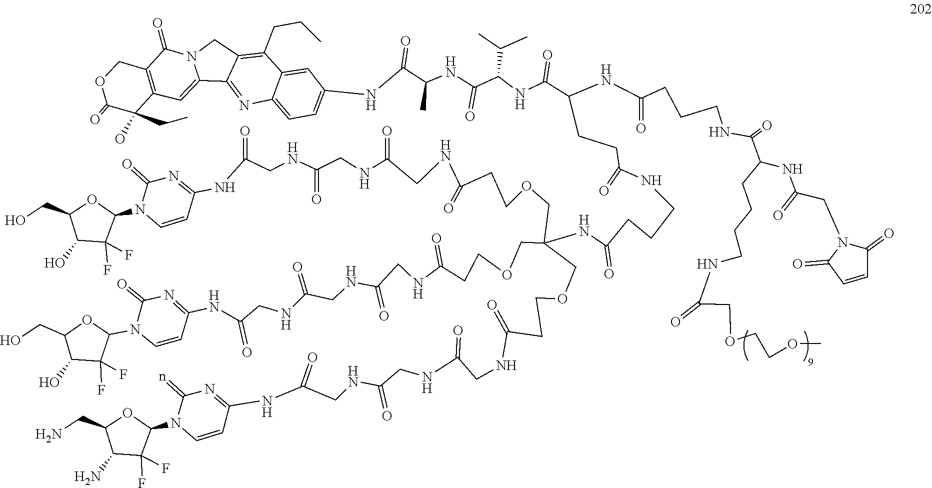

To a stirred solution of 201 (80 mg, 0.048 mmol, 1.40 eq) in DMF (1 mL) at room temperature were added HATU (18 mg, 0.048 mmol, 1.40 eq), 155 (52 mg, 0.034 mmol, 1.00 eq) and DIEA (31 μl, 0.185 mmol, 5.40 eq) in sequence. The resulting mixture was stirred at room temperature for 1 h. Prep-HPLC purification and lyophilization afforded 202 (4 mg, 3.6% yield) as a white solid. ESI-MS m/z: calcd. for C₁₃₅H₁₈₈F₆N₃₀O₅₂ [M+2H]²⁺ 1588.6447; found 1588.6447.

### Example 141. Synthesis of compound 204

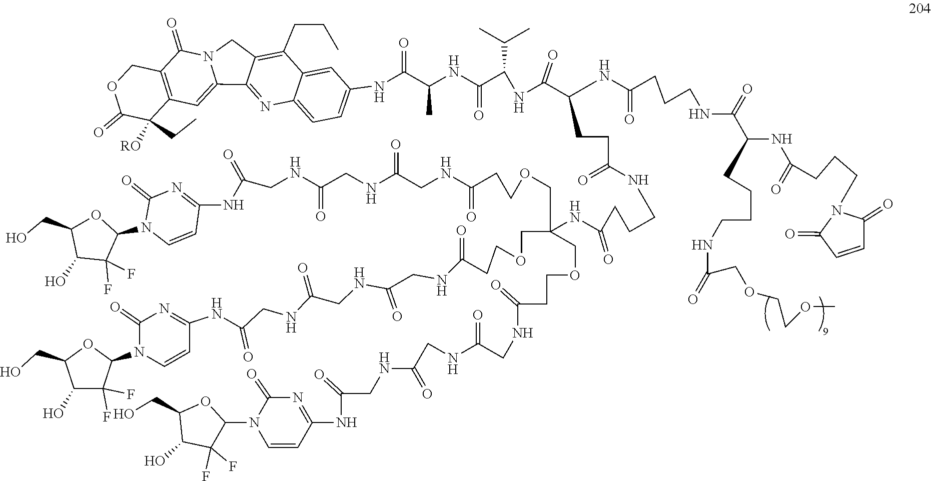

To a stirred solution of 140 (60.0 mg, 0.039 mmol) in DMF (1 mL) at room temperature were added 201 (84.74 mg, 0.051 mmol), HATU (22.05 mg, 0.058 mmol) and DIEA (25.20 mg, 0.195 mmol). The resulting mixture was stirred at room temperature for 30 min. before it was concentrated in vacuo. Prep-HPLC purification and lyophilization afforded 204 (25.0 mg, 20% o yield). ESI-MS m/z: calcd. for C₁₃₇H₁₉₃F₆N₃₀O₅₂ [M+H]⁺: 3204.32; found: 3204.32.

### Example 142. Synthesis of tert-butyl ((S)-1-(((R)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-3-methyl-1-oxobutan-2-yl)carbamate (207)

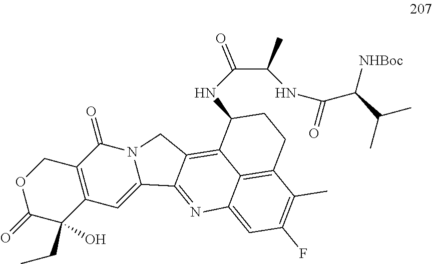 [SMILES: CC[C@@]1(O)C(=O)OCc2c1cc1n(c2=O)Cc2c-1nc1cc(F)c(C)c3c1c2[C@@H](NC(=O)[C@@H](C)NC(=O)[C@@H](NC(=O)OC(C)(C)C)C(C)C)CC3]

To a stirred solution of 206 (506 mg, 1.00 mmol) in DMF (10 mL) at room temperature were added (tert-butoxycarbonyl)-L-valine (217 mg, 1.00 mmol), HATU (456 mg, 1.20 mmol) and DIEA (387 mg, 3.00 mmol). The resulting mixture was stirred at room temperature for 30 min. before it was concentrated in vacuo. Prep-HPLC purification and lyophilization afforded 207 (621 mg, 88% yield).

### Example 142. Synthesis of (S)-2-amino-N—((R)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)-3-methylbutanamide (208)

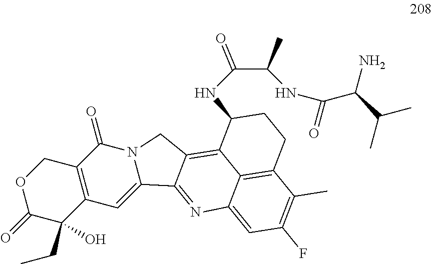 [SMILES: CC[C@@]1(O)C(=O)OCc2c1cc1n(c2=O)Cc2c-1nc1cc(F)c(C)c3c1c2[C@@H](NC(=O)[C@@H](C)NC(=O)[C@@H](N)C(C)C)CC3]

To a stirred solution of 207 (621 mg, 0.88 mmol) in DCM (8 mL) at room temperature was added TFA (8 mL). The resulting mixture was stirred at room temperature for 3 h, concentrated in vacuo to afford 208 (533 mg, 100% yield) as a white solid.

### Example 144. Synthesis of tert-butyl (37S, 45S)-37-(4-(2, 5-dioxo-2, 5-dihydro-1H-pyrrol-1-yl) butanamido)-45-(((2S)-1-(((2R)-1-(((1S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-1-oxopropan-2-yl)amino)-3-methyl-1-oxobutan-2-yl) carbamoyl)-31, 38, 43-trioxo-2, 5, 8, 11, 14, 17, 20, 23, 26, 29-decaoxa-32, 39, 44-triazaoctatetracontan-48-oate (209)

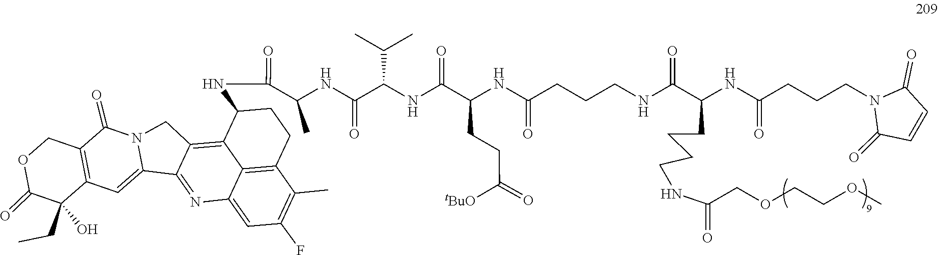

To a stirred solution of 208 (121 mg, 0.20 mmol) in DMF (1 mL) at room temperature were added 127 (210 mg, 0.20 mmol), HATU (91 mg, 0.24 mmol) and DIEA (65 mg, 0.50 mmol). The resulting mixture was stirred at room temperature for 30 min. before it was concentrated in vacuo. Prep-HPLC purification and lyophilization afforded 209 (259 mg, 79% yield).

### Example 145. Synthesis of (37S, 45S)-37-(4-(2, 5-dioxo-2, 5-dihydro-1H-pyrrol-1-yl) butanamido)-45-(((2S)-1-(((2R)-1-(((1S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-1-oxopropan-2-yl)amino)-3-methyl-1-oxobutan-2-yl) carbamoyl)-31, 38, 43-trioxo-2, 5, 8, 11, 14, 17, 20, 23, 26, 29-decaoxa-32, 39, 44-triazaoctatetracontan-48-oic acid (210)

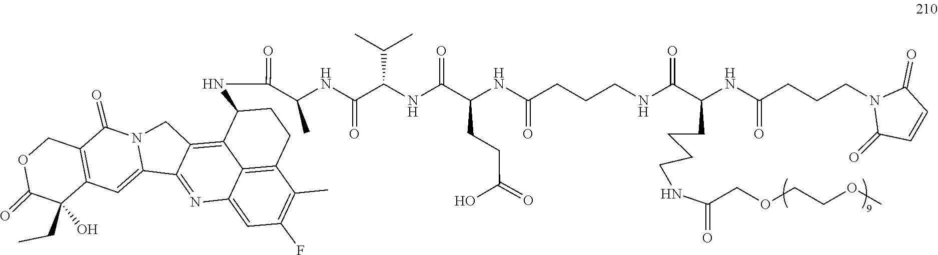

A solution of 209 (250.0 mg, 0.141 mmol) in DCM (3 mL) and TFA (1 mL) was stirred at room temperature for 1 h. The reaction was concentrated in vacuo to afford compound 210 (190.0 mg, 76% yield). ESI-MS m/z: [M+H]⁺ calcd. for C₇₆H₁₁₀FN₁₀O₂₅ 1581.75; found 1581.75.

### Example 146. Synthesis of compound 211

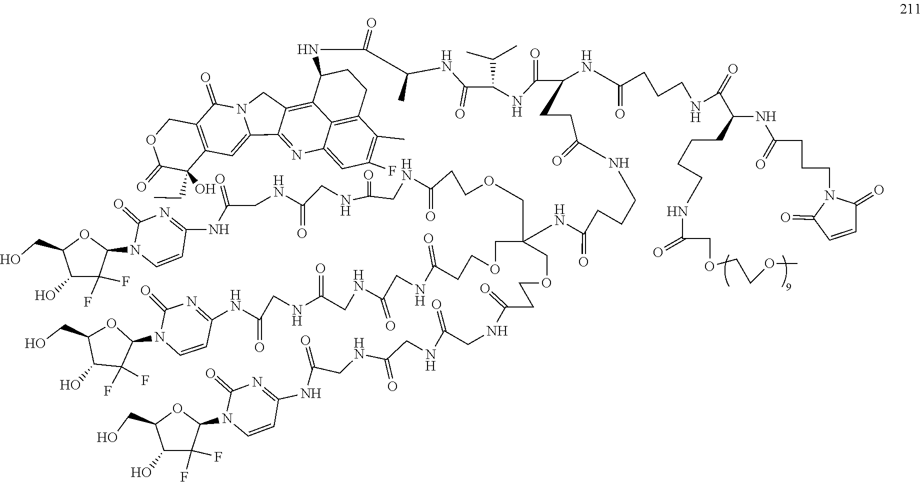

To a stirred solution of 210 (95.0 mg, 0.057 mmol) in DMF (2 mL) at room temperature were added 201 (84.74 mg, 0.051 mmol), HATU (21.61 mg, 0.057 mmol) and DIEA (11.02 mg, 0.085 mmol). The resulting mixture was stirred at room temperature for 20 min. before it was concentrated in vacuo. Prep-HPLC purification and lyophilization afforded 211 (12.6 mg, 7% yield) ESI-MS m/z: calcd. for C₁₃₈H₂₀₁F₇N₃₀O₅₂ [M+H]⁺ 3234.31; found 3234.31.

### Example 147. Synthesis of compound (213)

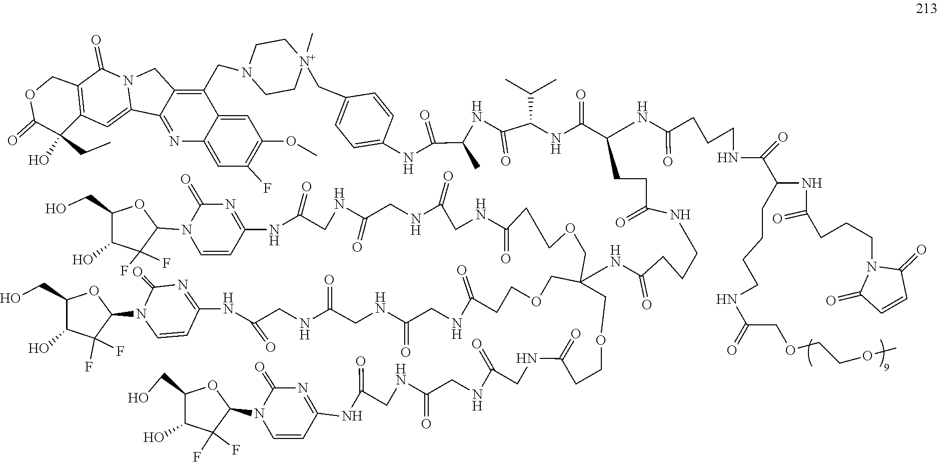

To a stirred solution of 189 (66.0 mg, 0.038 mmol) in DMF (2 mL) at room temperature were added 201 (70.0 mg, 0.042 mmol), DIEA (8.12 mg, 0.063 mmol) and HATU (16.01 mg, 0.042 mmol). The resulting mixture was stirred at room temperature for 15 min. before it was concentrated in vacuo. Prep-HPLC purification and lyophilization afforded 213 (19.6 mg, 14% yield). ESI-MS m/z: calcd. for C₁₄₈H₂₀₅F₇N₃₂O₅₃ [M+H]⁺ 3411.42; found 3411.42.

### Example 148. Synthesis of 1-(4-((S)-2-((tert-butoxycarbonyl)amino) propanamido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (216)

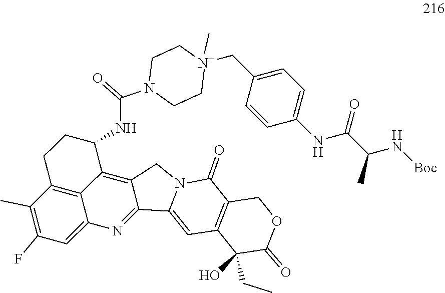 [SMILES: CC[C@@]1(O)C(=O)OCc2c1cc1n(c2=O)Cc2c-1nc1cc(F)c(C)c3c1c2[C@@H](NC(=O)N1CC[N+](C)(Cc2ccc(NC(=O)[C@H](C)NC(=O)OC(C)(C)C)cc2)CC1)CC3]

To a stirred solution of 215 (3.5 g, 6.584 mmol) in DMF (35 mL) at 0° C. were added DIEA (2.55 g, 19.753 mmol) and compound 112 (1.33 g, 6.584 mmol). After 15 min., compound 182 (2.61 g, 6.914 mmol) was added. The resulting mixture was stirred at 0° C. for 10 min. before it was concentrated in vacuo. Prep-HPLC purification and lyophilization afforded 216 (1.2 g, 42% yield). ESI-MS m/z: calcd. for C₄₅H₅₃FN₇O₈[M]⁺ 838.39; found 838.39.

### Example 149. Synthesis of 1-(4-((S)-2-aminopropanamido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (217)

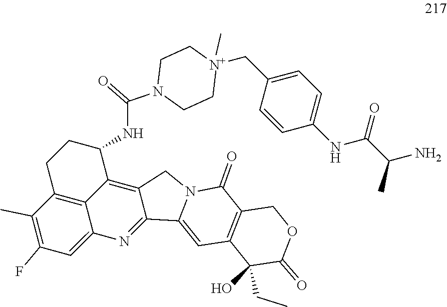 [SMILES: CC[C@@]1(O)C(=O)OCc2c1cc1n(c2=O)Cc2c-1nc1cc(F)c(C)c3c1c2[C@@H](NC(=O)N1CC[N+](C)(Cc2ccc(NC(=O)[C@H](C)N)cc2)CC1)CC3]

A solution of 216 (200.0 mg, 0.238 mmol) in DCM (3 mL) and TFA (1 mL) was stirred at room temperature for 1 h before it was concentrated in vacuo. Prep-HPLC purification and lyophilization afforded 217 (154.6 mg, 88% yield). ESI-MS m/z: calcd. for C₄₀H₄₅FN₇O₆ [M]⁺ 738.34; found 738.34.

### Example 150. Synthesis of 1-(4-((S)-2-((S)-2-((tert-butoxycarbonyl)amino)-3-methylbutanamido) propanamido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (218)

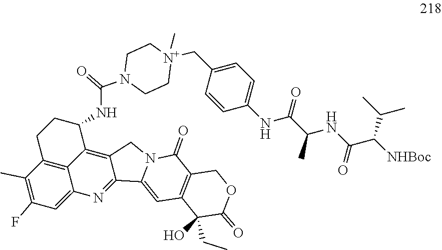 [SMILES: CC[C@@]1(O)C(=O)OCc2c1cc1n(c2=O)Cc2c-1nc1cc(F)c(C)c3c1c2[C@@H](NC(=O)N1CC[N+](C)(Cc2ccc(NC(=O)[C@H](C)NC(=O)[C@@H](NC(=O)OC(C)(C)C)C(C)C)cc2)CC1)CC3]

To a stirred solution of 217 (739 mg, 1.00 mmol) in DMF (10 mL) at room temperature were added (tert-butoxycarbonyl)-L-valine (217 mg, 1.00 mmol), HATU (456 mg, 1.20 mmol) and DIEA (387 mg, 3.00 mmol). The resulting mixture was stirred at room temperature for 30 min. before it was concentrated in vacuo. Prep-HPLC purification and lyophilization afforded 218 (844 mg, 90% yield).

### Example 151. Synthesis of 1-(4-((S)-2-((S)-2-amino-3-methylbutanamido) propanamido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (219)

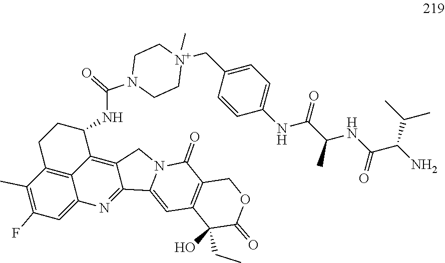 [SMILES: CC[C@@]1(O)C(=O)OCc2c1cc1n(c2=O)Cc2c-1nc1cc(F)c(C)c3c1c2[C@@H](NC(=O)N1CC[N+](C)(Cc2ccc(NC(=O)[C@H](C)NC(=O)[C@@H](N)C(C)C)cc2)CC1)CC3]

To a stirred solution of 218 (844 mg, 0.90 mmol) in DCM (20 mL) at room temperature was added TFA (20 mL). The resulting mixture was stirred at room temperature for 3 h, concentrated in vacuo to afford 219 (754 mg, 100% yield) as a white solid.

### Example 152. Synthesis of 1-(4-((37S, 45S, 48S, 51S)-45-(3-(tert-butoxy)-3-oxopropyl)-37-(4-(2, 5-dioxo-2, 5-dihydro-1H-pyrrol-1-yl) butanamido)-48-isopropyl-51-methyl-31, 38, 43, 46, 49-pentaoxo-2, 5, 8, 11, 14, 17, 20, 23, 26, 29-decaoxa-32, 39, 44, 47, 50-pentaazadopentacontan-52-amido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (220)

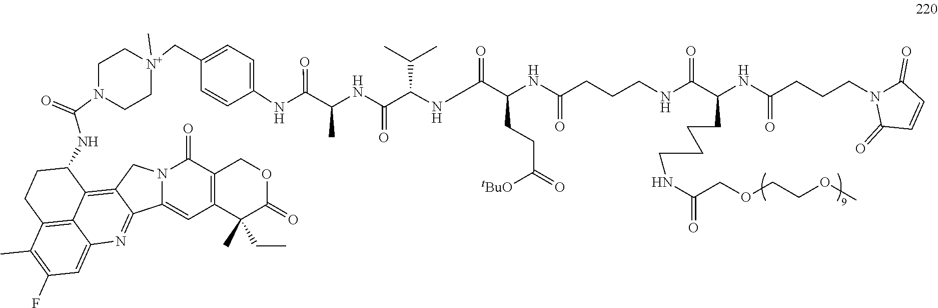

To a stirred solution of 219 (168 mg, 0.20 mmol) in DMF (1 mL) at room temperature were added 127 (210 mg, 0.20 mmol), HATU (91 mg, 0.24 mmol) and DIEA (65 mg, 0.50 mmol). The resulting mixture was stirred at room temperature for 30 min. before it was concentrated in vacuo. Prep-HPLC purification and lyophilization afforded 220 (280 mg, 75% yield).

### Example 153. Synthesis of 1-(4-((13S, 21S, 24S, 27S)-21-(2-carboxyethyl)-13-(4-(2, 5-dioxo-2, 5-dihydro-1H-pyrrol-1-yl) butanamido)-24-isopropyl-27-methyl-7, 14, 19, 22, 25-pentaoxo-2, 5-dioxa-8, 15, 20, 23, 26-pentaazaoctacosan-28-amido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (221)

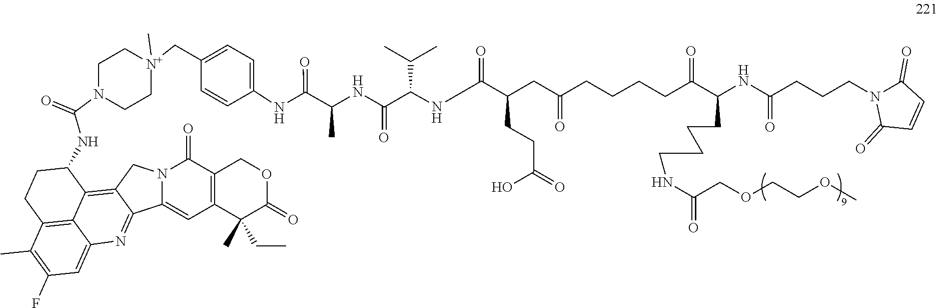

A solution of 220 (180.0 mg, 0.091 mmol) in DCM (2 mL) and TFA (1 mL) was stirred at room temperature for 1 h before it was concentrated in vacuo. Prep-HPLC purification and lyophilization afforded 221 (125.0 mg, 72% yield). ESI-MS m/z: calcd. for Cs₉H₁₂₇FN₁₃O₂₆ [M+H]⁺ 1813.90; found 1813.90.

### Example 154. Synthesis of compound 222

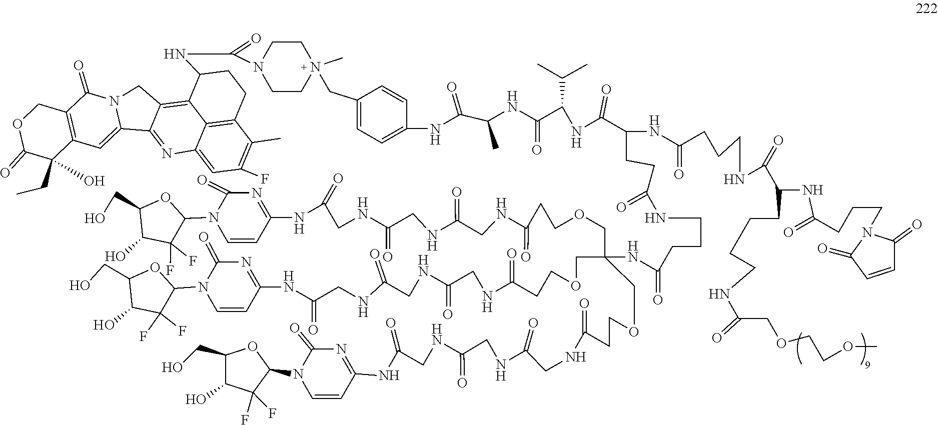

To a stirred solution of 221 (20.0 mg, 0.011 mmol) in DMF (1 mL) at room temperature were added 201 (23.61 mg, 0.013 mmol), HATU (8.38 mg, 0.022 mmol) and DIEA (8.55 mg, 0.066 mmol). The resulting mixture was stirred at room temperature for 15 min. before it was concentrated in vacuo. Prep-HPLC purification and lyophilization afforded 222 (7.7 mg, 20% yield). ESI-MS m/z: calcd. for C₁₅₁ H₂₀₉F₇N₃₃O₅₃ [M+3 H]⁺ 1155.82; found 1156.31.

### Example 155. tert-butyl (2S, 4R)-4-((tert-butoxycarbonyl)amino)-5-(4-(((5S, 8S, 11S, 14S, 31S, 34S, 37S, 40S)-44-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-22, 23-bis (2, 5-dioxo-2, 5-dihydro-1H-pyrrol-1-yl)-14, 31-bis(4-(2-(2-methoxyethoxy) acetamido)butyl)-5, 8, 11, 34, 37, 40-hexamethyl-4, 7, 10, 13, 16, 21, 24, 29, 32, 35, 38, 41-dodecaoxo-3, 6, 9, 12, 15, 20, 25, 30, 33, 36, 39, 42-dodecaazatetratetracontyl)oxy)phenyl)-2-methylpentanoate (226)

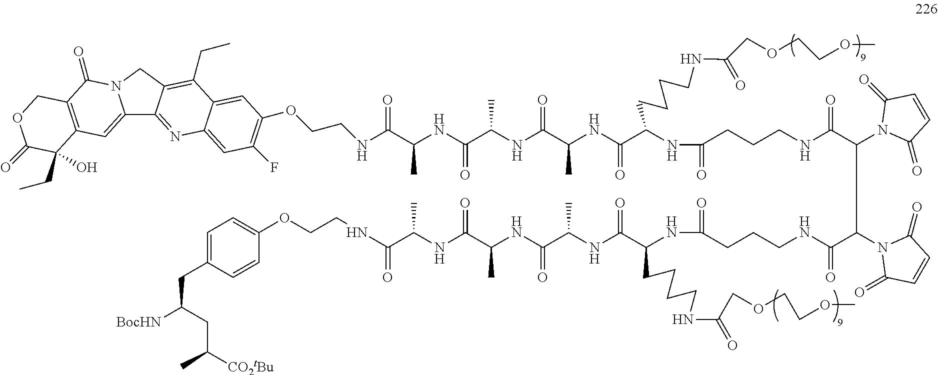

To a stirred solution of 224 (253.9 mg, 0.11 mmol) in DMF (4 mL) at room temperature were added HATU (41 mg, 0.11 mmol) and DIEA (40 pl, 0.22 mmol). The reaction mixture was stirred at room temperature for 0.5 h. 225 (47 mg, 0.11 mmol) was added and the reaction mixture was stirred for 3 h at room temperature, purified by prep-HPLC, lyophilized to afford 226 (36.2 mg, 11% yield).

### Example 156. (2S, 4R)-4-amino-5-(4-(((5S, 8S, 11S, 14S, 31S, 34S, 37S, 40S)-44-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-22, 23-bis (2, 5-dioxo-2, 5-dihydro-1H-pyrrol-1-yl)-14, 31-bis(4-(2-(2-methoxyethoxy) acetamido)butyl)-5, 8, 11, 34, 37, 40-hexamethyl-4, 7, 10, 13, 16, 21, 24, 29, 32, 35, 38, 41-dodecaoxo-3, 6, 9, 12, 15, 20, 25, 30, 33, 36, 39, 42-dodecaazatetratetracontyl)oxy)phenyl)-2-methylpentanoic acid (227)

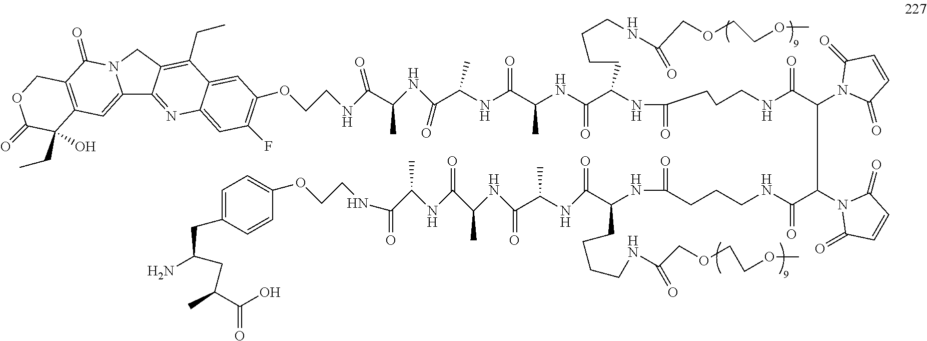

To a stirred solution of 226 (36.2 mg, 0.012 mmol) in DCM (1 mL) at room temperature were added TFA (1 mL). The reaction mixture was stirred at room temperature for 1 h, concentrated to afford 227 (30 mg, 100% yield).

### Example 157. (2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(4-(((5S, 8S, 111S, 14S, 31S, 34S, 37S, 40S)-44-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-22, 23-bis (2, 5-dioxo-2, 5-dihydro-1H-pyrrol-1-yl)-14, 31-bis(4-(2-(2-methoxyethoxy) acetamido)butyl)-5, 8, 11, 34, 37, 40-hexamethyl-4, 7, 10, 13, 16, 21, 24, 29, 32, 35, 38, 41-dodecaoxo-3, 6, 9, 12, 15, 20, 25, 30, 33, 36, 39, 42-dodecaazatetratetracontyl)oxy)phenyl)-2-methylpentanoic acid (228)

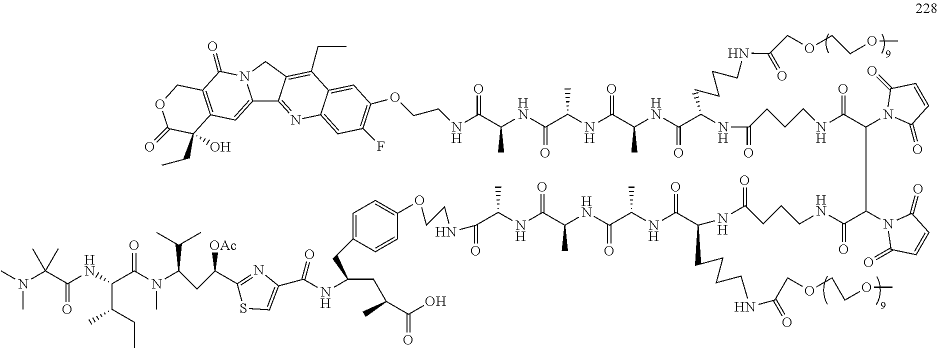

To a stirred solution of 227 (33.39 mg, 0.012 mmol) in DMF (2 mL) at room temperature were added 24 (8.31 mg, 0.012 mmol) and DIEA (6.2 μL, 0.048 mm). The reaction mixture was stirred at room temperature for 2 h, purified by prep-HPLC, lyophilized to afford 228 (21 mg, 53% yield).

### Example 158. (S)-4-(2-((tert-butoxycarbonyl)amino) propanamido)benzyl hydrazinecarboxylate (230)

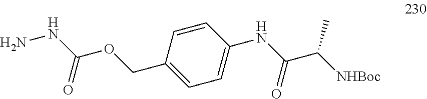 [SMILES: C[C@H](NC(=O)OC(C)(C)C)C(=O)Nc1ccc(COC(=O)NN)cc1]

To a stirred solution of 178 (1.0 g, 2.54 mmol) in DCM (30 mL) were added 112 (0.6 g, 3.05 mmol), DMAP (0.3 g, 2.54 mmol) and DIEA (1.3 g, 10.166 mmol) at room temperature. The reaction mixture was stirred overnight and concentrated, purified by a silica gel column, eluted with DCM and methanol, to afford 230 (730 mg, 63% yield).

### Example 159. 4—((S)-2-((tert-butoxycarbonyl)amino) propanamido)benzyl 2-((2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-2-methyl-5-phenylpentanoyl) hydrazine-1-carboxylate (232)

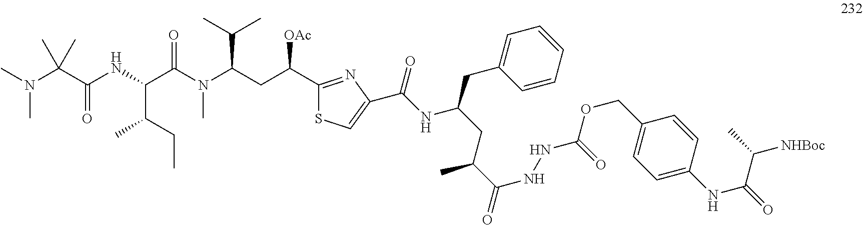 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccccc2)C[C@H](C)C(=O)NNC(=O)OCc2ccc(NC(=O)[C@H](C)NC(=O)OC(C)(C)C)cc2)cs1)C(C)C]

To a stirred solution of 231 (274 mg, 0.393 mmol) in DMF (5 mL) at room temperature were added 230 (173 mg, 0.383 mmol), HATU (218 mg, 0.575 mmol) and DIEA (99 mg, 0.766 mmol). The reaction mixture was stirred at room temperature for 2 h, purified by prep-HPLC, lyophilized to afford 232 (130 mg, 29% yield).

### Example 160. 4—((S)-2-aminopropanamido)benzyl 2-((2S, 4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-2-methyl-5-phenylpentanoyl) hydrazine-1-carboxylate (233)

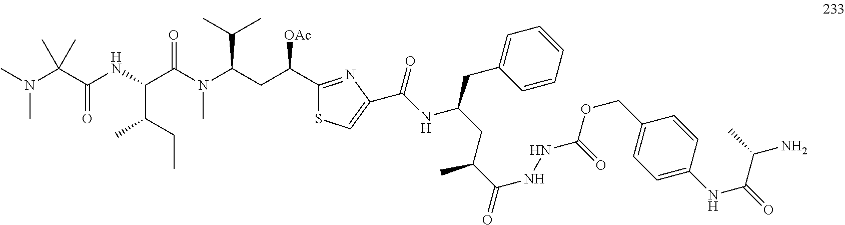 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccccc2)C[C@H](C)C(=O)NNC(=O)OCc2ccc(NC(=O)[C@H](C)N)cc2)cs1)C(C)C]

To a stirred solution of 232 (130 mg, 0.113 mmol) in DCM (3 mL) at room temperature was added TFA (3 mL). The reaction mixture was stirred at room temperature for 2 h, concentrated to afford 233 (118 mg, 100% yield).

### Example 161. N2-((13S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-7, 15, 20, 23-tetraoxo-2, 5-dioxa-8, 14, 19, 24-tetraazaoctacosan-28-oyl)-N6-(2-(2-methoxyethoxy) acetyl)-L-lysyl-L-valyl-L-alanine (235)

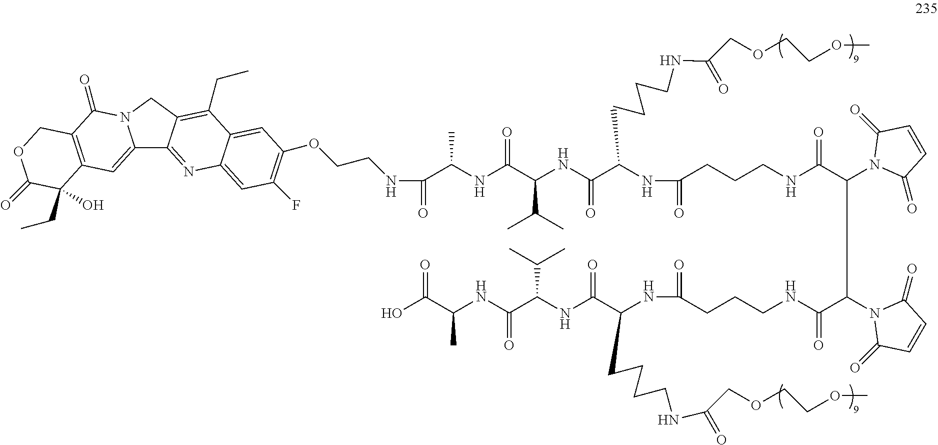

To a stirred solution of 234 (235.0 mg, 0.112 mmol) in DMF (3 mL) at room temperature were added HATU (43 mg, 0.112 mmol) and DIEA (29 mg, 0.224 mmol). After 15 min., compound 128 (50.8 mg, 0.112 mmol) was added and the reaction mixture was stirred at room temperature for 1 h to afford a solution of 235, which was used directly in the next step.

### Example 162. 4-((13S, 30S, 33S, 36S, 39S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-33-isopropyl-30-(4-(2-(2-methoxyethoxy) acetamido)butyl)-36, 39-dimethyl-7, 15, 20, 23, 28, 31, 34, 37-octaoxo-2, 5-dioxa-8, 14, 19, 24, 29, 32, 35, 38-octaazatetracontan-40-amido)benzyl 2-((2S, 3S)-3-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-2-methyl-4-phenylbutanoyl) hydrazine-1-carboxylate (236)

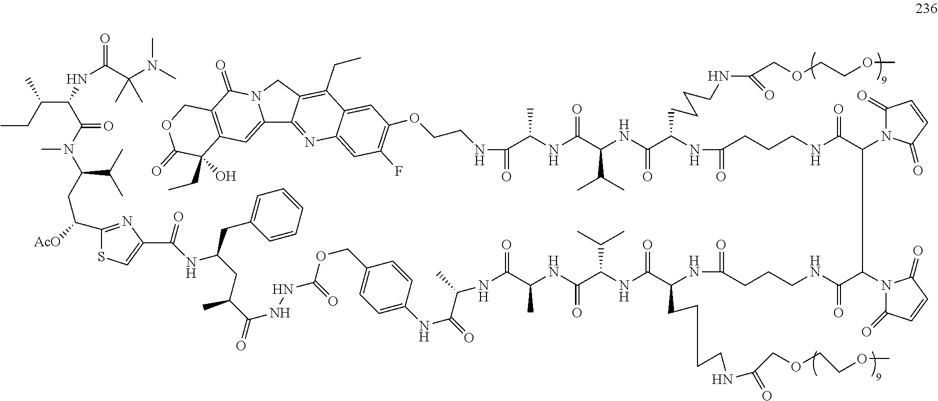

To a stirred solution of 235 at room temperature were added 233 (118 mg, 0.112 mmol), HATU (43 mg, 0.112 mmol) and DIEA (29 mg, 0.224 mmol). The reaction mixture was stirred at room temperature for 2 h, purified by prep-HPLC, lyophilized to afford 236 (15 mg, 4% yield).

### Example 163. tert-butyl (4R)-4-((tert-butoxycarbonyl)amino)-5-(3-((13S, 30S, 33S, 36S, 39S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-33-isopropyl-30-(4-(2-(2-methoxyethoxy) acetamido)butyl)-36, 39-dimethyl-7, 15, 20, 23, 28, 31, 34, 37-octaoxo-2, 5-dioxa-8, 14, 19, 24, 29, 32, 35, 38-octaazatetracontan-40-amido)-4-hydroxyphenyl) pentanoate (238)

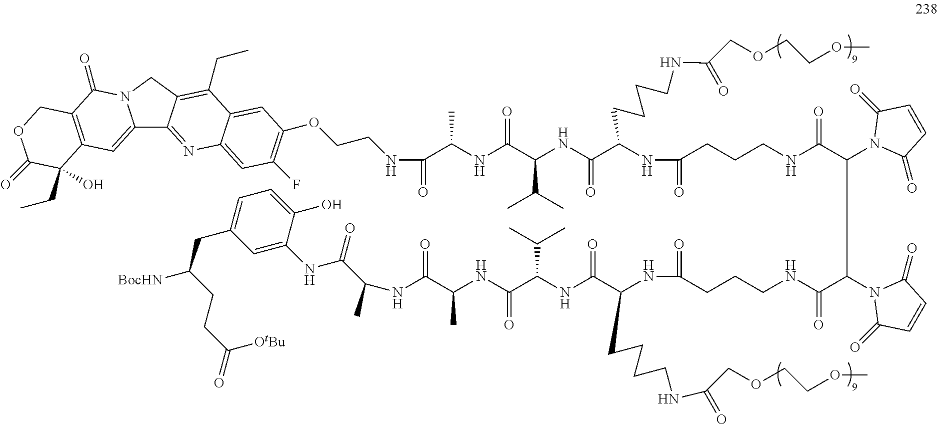

To a stirred solution of 235 (300 mg, 0.123 mmol) in DMF (5 mL) at room temperature was added HATU (65 mg, 0.172 mmol). The reaction mixture was stirred at room temperature for 10 min., and compound 21 (61 mg, 0.135 mmol) and DIEA (32 mg) were added. After being stirred at room temperature for 1 h, the reaction mixture was purified by prep-HPLC, lyophilized to afford 238 (154 mg, 44% yield).

### Example 164. (4R)-4-amino-5-(3-((13S, 30S, 33S, 36S, 39S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-33-isopropyl-30-(4-(2-(2-methoxyethoxy) acetamido)butyl)-36, 39-dimethyl-7, 15, 20, 23, 28, 31, 34, 37-octaoxo-2, 5-dioxa-8, 14, 19, 24, 29, 32, 35, 38-octaazatetracontan-40-amido)-4-hydroxyphenyl) pentanoic acid (239)

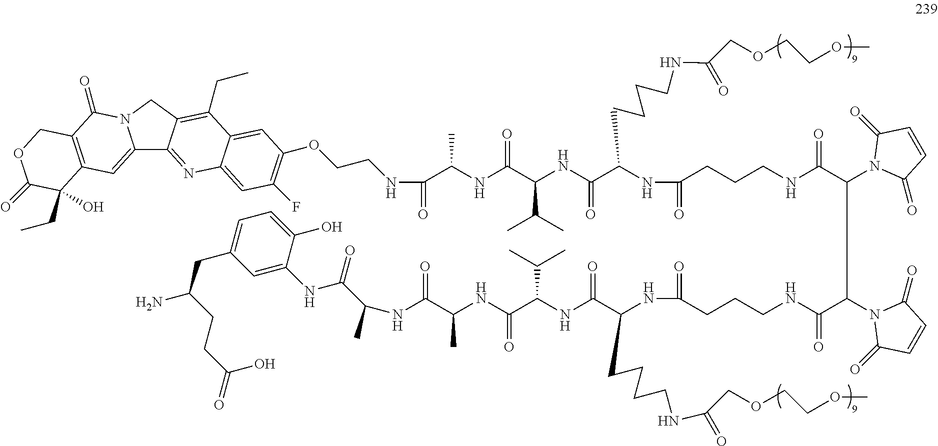

To a stirred solution of 238 (70 mg, 0.024 mmol) in DCM (3 mL) at room temperature was added TFA (3 mL). The reaction mixture was stirred at room temperature for 1 h, concentrated to afford 239 (66 mg, 100% yield).

### Example 165. (4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((13S, 30S, 33S, 36S, 39S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-33-isopropyl-30-(4-(2-(2-methoxyethoxy) acetamido)butyl)-36, 39-dimethyl-7, 15, 20, 23, 28, 31, 34, 37-octaoxo-2, 5-dioxa-8, 14, 19, 24, 29, 32, 35, 38-octaazatetracontan-40-amido)-4-hydroxyphenyl) pentanoic acid (240)

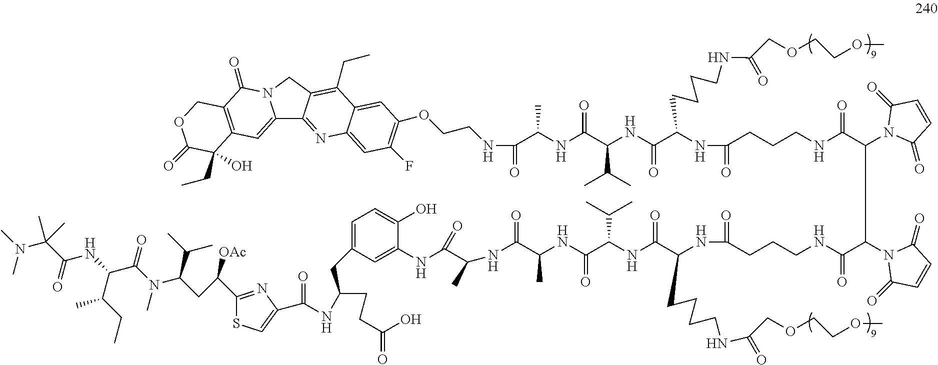

To a stirred solution of 239 (66 mg, 0.024 mmol) in DMF (4 mL) at room temperature were added 24 (17 mg, 0.024 mmol) and DIEA (12.5 mg, 0.097 mmol). The reaction mixture was stirred at room temperature for 2 h, purified by prep-HPLC, lyophilized to afford 240 (23 mg, 30% yield).

### Example 166. N1-((5S, 8S, 1 1S, 14S)-1-((4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-14-(4-(2-(2-methoxyethoxy) acetamido)butyl)-5, 8, 1 1-trimethyl-4, 7, 10, 13, 16-pentaoxo-3, 6, 9, 12, 15-pentaazanonadecan-19-yl)-2, 3-bis (2, 5-dioxo-2, 5-dihydro-1H-pyrrol-1-yl)-N4-((13S)-13-(((2S)-1-(((2S)-1-(((2S)-1-(((1S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-1-oxopropan-2-yl)amino)-1-oxopropan-2-yl)amino)-1-oxopropan-2-yl) carbamoyl)-7, 15-dioxo-2, 5-dioxa-8, 14-diazaoctadecan-18-yl) succinamide (242)

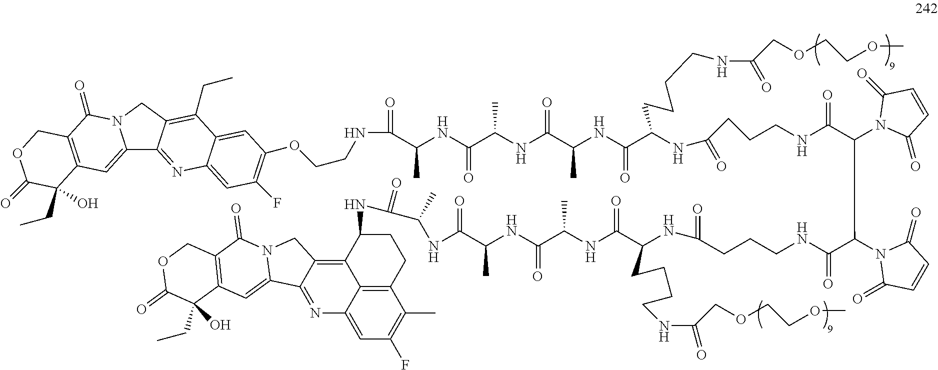

To a stirred solution of 224 (750 mg, 0.36 mmol) in DMF (5 mL) at room temperature was added HATU (137 mg, 0.36 mmol). After stirring at room temperature for 10 min., compound 128 (164 mg, 0.36 mmol) and DIEA (56 mg, 0.432 mmol) were added. The reaction mixture was further stirred at room temperature for 2 h and compound 215 (11 mg, 0.02 mmol) was added. After being stirred for 2 h, the reaction mixture was purified by prep-HPLC, lyophilized to afford 242 (7 mg, 12% yield).

### Example 167. N1-((13S)-13-(((2S)-1-(((2S)-1-((2-((4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-7, 15-dioxo-2, 5-dioxa-8, 14-diazaoctadecan-18-yl)-2, 3-bis (2, 5-dioxo-2, 5-dihydro-1H-pyrrol-1-yl)-N4-((13S)-13-(((2S)-1-(((2S)-1-(((1S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-1-oxopropan-2-yl)amino)-3-methyl-1-oxobutan-2-yl) carbamoyl)-7, 15-dioxo-2, 5-dioxa-8, 14-diazaoctadecan-18-yl) succinamide (244)

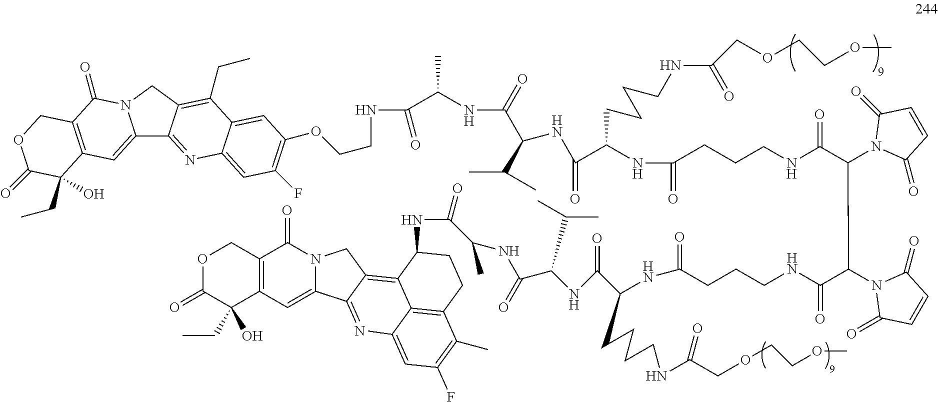

To a stirred solution of 235 (80 mg, 0.033 mmol) in DMF (3 mL) at room temperature was added HATU (18.7 mg, 0.049 mmol). The reaction mixture was stirred at room temperature for 4 min. A solution of 215 (21 mg, 0.039 mmol) in DMF (2 mL) at room temperature was added, followed by DIEA (9 mg, 0.065 mmol). The reaction mixture was stirred for 0.5 h at room temperature, and then purified by prep-HPLC, lyophilized to afford 244 (34 mg, 36% yield).

### Example 168. tert-butyl (4R)-4-((tert-butoxycarbonyl)amino)-5-(3-((13S, 30S, 33S, 36S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[I, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-33-isopropyl-30-(4-(2-(2-methoxyethoxy) acetamido)butyl)-36-methyl-7, 15, 20, 23, 28, 31, 34, 37-octaoxo-2, 5-dioxa-8, 14, 19, 24, 29, 32, 35, 38-octaazadotetracontan-42-amido)-4-hydroxyphenyl) pentanoate (246)

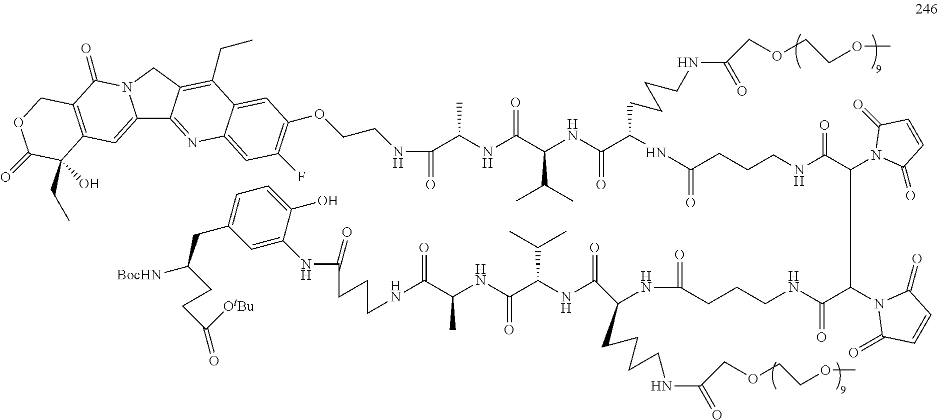

To a stirred solution of 235 (300 mg, 0.123 mmol) in DMF (5 mL) at room temperature was added HATU (65 mg, 0.172 mmol). The reaction mixture was stirred at room temperature for 10 min., and 79 (61 mg, 0.135 mmol) and DIEA (32 mg) were added. After being stirred at room temperature for 1 h, the reaction mixture was purified by prep-HPLC, lyophilized to afford 246 (154 mg, 44% yield).

### Example 169. (4R)-4-amino-5-(3-((13S, 30S, 33S, 36S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-IH-pyrrol-1-yl)-33-isopropyl-30-(4-(2-(2-methoxyethoxy) acetamido)butyl)-36-methyl-7, 15, 20, 23, 28, 31, 34, 37-octaoxo-2, 5-dioxa-8, 14, 19, 24, 29, 32, 35, 38-octaazadotetracontan-42-amido)-4-hydroxyphenyl) pentanoic acid (247)

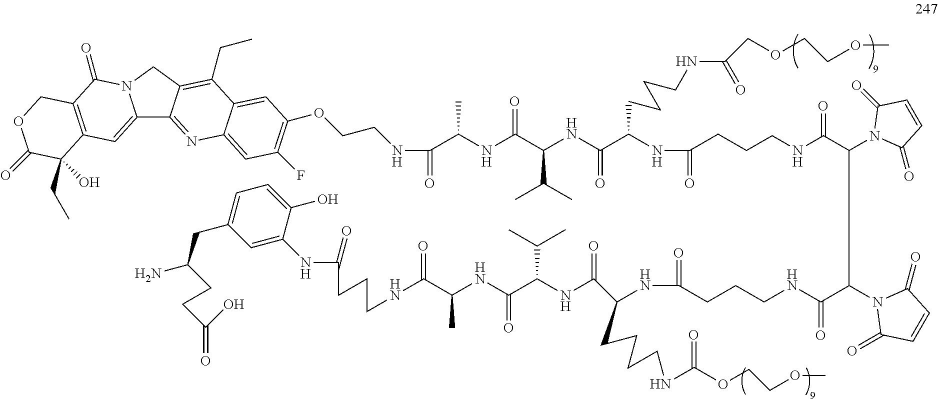

To a stirred solution of 246 (70 mg, 0.024 mmol) in DCM (3 mL) at room temperature was added TFA (3 mL). The reaction mixture was stirred at room temperature for 1 h, and then concentrated to afford 247 (66 mg, 100% yield).

### Example 170. (4R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((13S, 30S, 33S, 36S)-13-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-33-isopropyl-30-(4-(2-(2-methoxyethoxy) acetamido)butyl)-36-methyl-7, 15, 20, 23, 28, 31, 34, 37-octaoxo-2, 5-dioxa-8, 14, 19, 24, 29, 32, 35, 38-octaazadotetracontan-42-amido)-4-hydroxyphenyl) pentanoic acid (248)

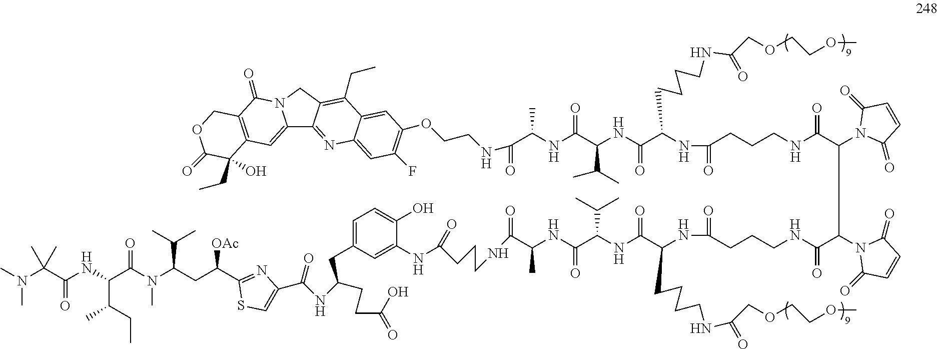

To a stirred solution of 247 (235 mg, 0.09 mmol) in DMF (5 mL) at room temperature were added 24 (75 mg, 0.09 mmol) and DIEA (24 mg, 0.180 mmol). The reaction mixture was stirred at room temperature for 1 h, purified by prep-HPLC, lyophilized to afford 248 (9 mg, 3% yield).

### Example 171. N2-((13S)-13-(((S)-1-(((S)-1-((2-((5-((R)-5-(tert-butoxy)-2-((tert-butoxycarbonyl)amino)-5-oxopentyl)-2-hydroxyphenyl)amino)-2-oxoethyl)amino)-1-oxopropan-2-yl)amino)-3-methyl-1-oxobutan-2-yl) carbamoyl)-21, 22-bis (2, 5-dioxo-2, 5-dihydro-1H-pyrrol-1-yl)-7, 15, 20, 23-tetraoxo-2, 5-dioxa-8, 14, 19, 24-tetraazaoctacosan-28-oyl)-N6-(2-(2-methoxyethoxy) acetyl)-L-lysyl-L-valyl-L-alanine (250)

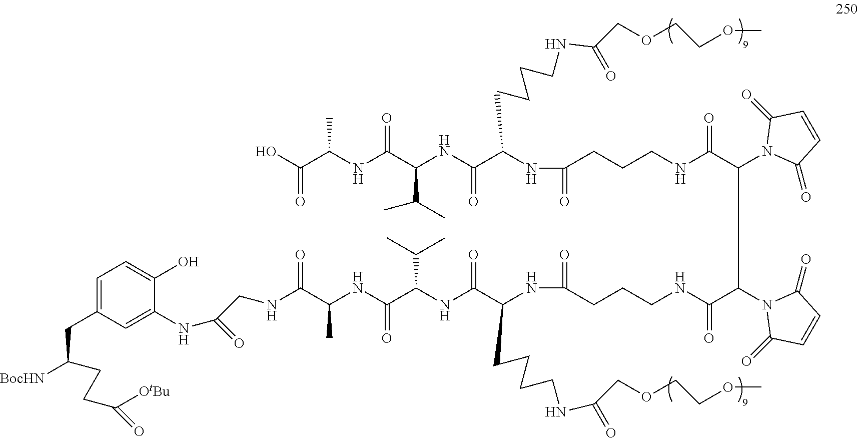

To a stirred solution of 234 (900 mg, 0.447 mmol) in DMF (6 mL) at room temperature was added HATU (170 mg, 0.447 mmol). After 10 min., 74 (196 mg, 0.447 mmol) and DIEA (58 mg, 0.447 mmol) were added and stirred at room temperature for 1 h. The reaction mixture was purified by prep-HPLC, lyophilized to afford 250 (184 mg, 17% yield).

### Example 172. tert-butyl (4R)-4-((tert-butoxycarbonyl)amino)-5-(3-((8S, 11S, 14S, 31S, 34S, 37S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-22, 23-bis (2, 5-dioxo-2, 5-dihydro-1H-pyrrol-1-yl)-11, 34-diisopropyl-14, 31-bis(4-(2-(2-methoxyethoxy) acetamido)butyl)-8, 37-dimethyl-4, 7, 10, 13, 16, 21, 24, 29, 32, 35, 38-undecaoxo-3, 6, 9, 12, 15, 20, 25, 30, 33, 36, 39-undecaazahentetracontan-41-amido)-4-hydroxyphenyl) pentanoate (252)

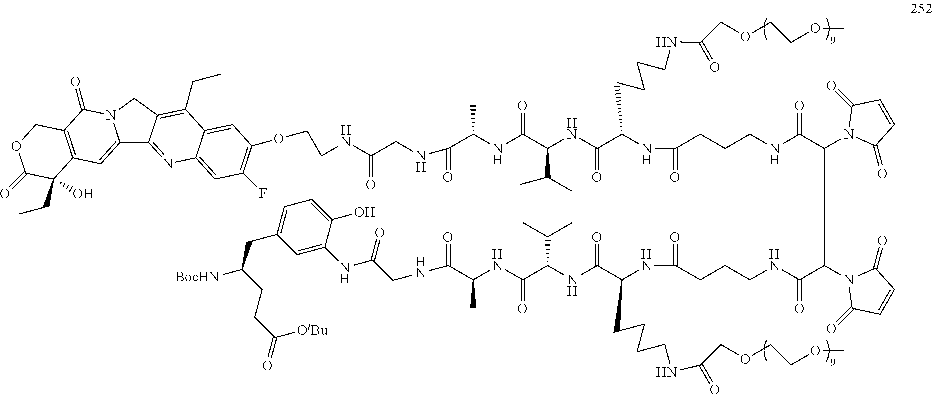

To a stirred solution of 250 (184 mg, 0.09 mmol) in DMF (3 mL) at room temperature was added HATU (34 mg, 0.09 mmol). After stirring at room temperature for 10 min., compound 251 (46 mg, 0.09 mmol) and DIEA (20 mg, 0.15 mmol) were added. The reaction mixture was stirred at room temperature for 1 h, purified by prep-HPLC, lyophilized to afford 252 (42 mg, 19% yield).

### Example 173. (4R)-4-amino-5-(3-((8S, 11S, 14S, 31S, 34S, 37S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-22, 23-bis (2, 5-dioxo-2, 5-dihydro-1H-pyrrol-1-yl)-11, 34-diisopropyl-14, 31-bis(4-(2-(2-methoxyethoxy) acetamido)butyl)-8, 37-dimethyl-4, 7, 10, 13, 16, 21, 24, 29, 32, 35, 38-undecaoxo-3, 6, 9, 12, 15, 20, 25, 30, 33, 36, 39-undecaazahentetracontan-41-amido)-4-hydroxyphenyl) pentanoic acid (253)

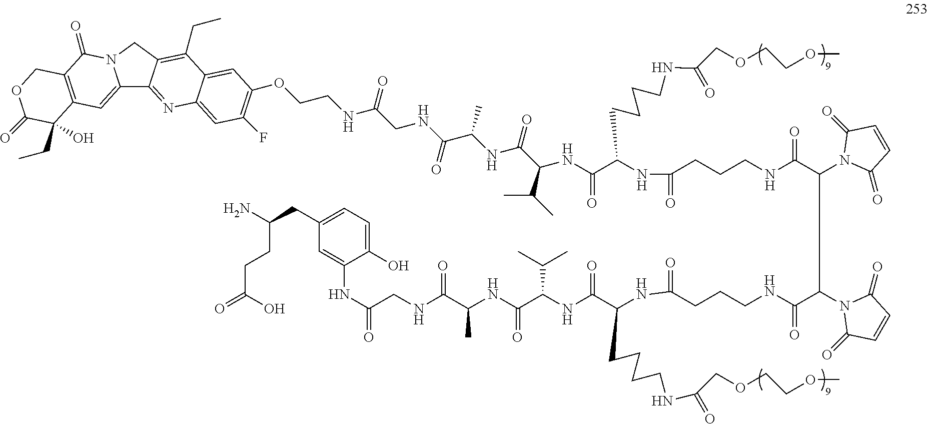

To a stirred solution of 252 (42 mg, 0.014 mmol) in DCM (2 mL) at room temperature was added TFA (4 mL). The reaction mixture was stirred at room temperature for 2 h, concentrated to afford 253 (40 mg, 100% yield).

### Example 174. (4R)-4-(2-((6S, 9R, 1 IR)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((8S, 1 IS, 14S, 31S, 34S, 37S)-1-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)-22, 23-bis (2, 5-dioxo-2, 5-dihydro-1H-pyrrol-1-yl)-11, 34-diisopropyl-14, 31-bis(4-(2-(2-methoxyethoxy) acetamido)butyl)-8, 37-dimethyl-4, 7, 10, 13, 16, 21, 24, 29, 32, 35, 38-undecaoxo-3, 6, 9, 12, 15, 20, 25, 30, 33, 36, 39-undecaazahentetracontan-41-amido)-4-hydroxyphenyl) pentanoic acid (254)

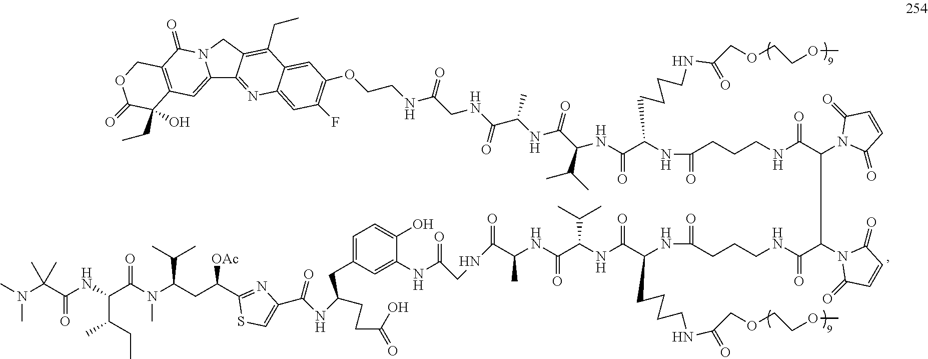

To a stirred solution of 253 (40 mg, 0.014 mmol) in DMF (5 mL) at room temperature were added 24 (11 mg, 0.016 mmol) and DIEA (40 mg, 0.309 mmol). The reaction mixture was stirred at room temperature for 1 h, purified by prep-HPLC, lyophilized to afford 254 (30 mg, 63% yield).

### Example 175. tert-butyl ((S)-1-(((S)-1-((2-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-2-oxoethyl)amino)-1-oxopropan-2-yl)amino)-3-methyl-1-oxobutan-2-yl) carbamate (257)

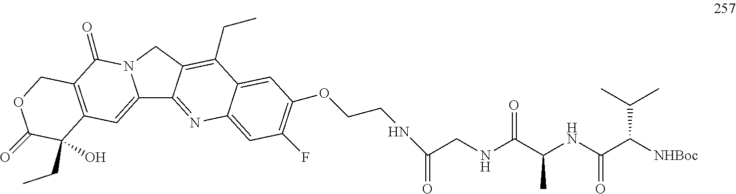 [SMILES: CCc1c2c(nc3cc(F)c(OCCNC(=O)CNC(=O)[C@H](C)NC(=O)[C@@H](NC(=O)OC(C)(C)C)C(C)C)cc13)-c1cc3c(c(=O)n1C2)COC(=O)[C@]3(O)CC]

To a stirred solution of 256 (1.98 g, 5.7 mmol) in DMF (100 mL) at room temperature were added HATU (2.35 g, 6.2 mmol), 128 (2.00 g, 4.4 mmol) and DIEA (1.14 g, 8.8 mmol). The reaction mixture was stirred at room temperature for 1 h and concentrated in vacuo. The residue was purified by a silica gel column, eluted with DCM and methanol, to afford 257 (3.18 g, 92% yield). ESI-MS m/z: [M+H]⁺ calcd. for C₃₉H₄₉FN₆O₁₀ 780.35; found 781.41.

### Example 176. (S)-2-amino-N—((S)-1-((2-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-2-oxoethyl)amino)-1-oxopropan-2-yl)-3-methylbutanamide (258)

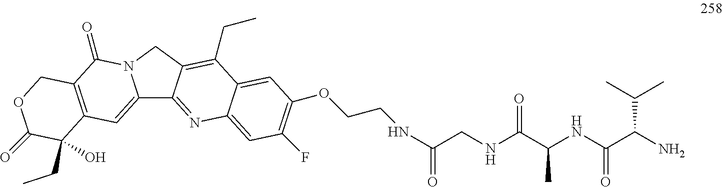 [SMILES: CCc1c2c(nc3cc(F)c(OCCNC(=O)CNC(=O)[C@H](C)NC(=O)[C@@H](N)C(C)C)cc13)-c1cc3c(c(=O)n1C2)COC(=O)[C@]3(O)CC]

To a stirred solution of 257 (3.18 g, 4.1 mmol) in DCM (10 mL) at room temperature was added TFA (5 mL). The reaction mixture was stirred at room temperature for 16 h, concentrated to afford 258 (2.2 g, 79% yield). ESI-MS m/z: [M+H]⁺calcd. for C₂₄H₂₄N₄O₅ 448.17; found 449.25.

### Example 177. benzyl (8S, 1 1S, 14S)-14-((tert-butoxycarbonyl)amino)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11-isopropyl-8-methyl-4, 7, 10, 13-tetraoxo-3, 6, 9, 12-tetraazaheptadecan-17-oate (260)

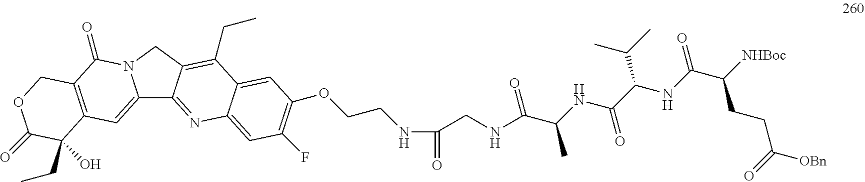 [SMILES: CCc1c2c(nc3cc(F)c(OCCNC(=O)CNC(=O)[C@H](C)NC(=O)[C@@H](NC(=O)[C@H](CCC(=O)OCc4ccccc4)NC(=O)OC(C)(C)C)C(C)C)cc13)-c1cc3c(c(=O)n1C2)COC(=O)[C@]3(O)CC]

To a stirred solution of 259 (0.55 g, 1.6 mmol) in DMF (100 mL) at room temperature were added HATU (2.35 g, 6.2 mmol), 128 (2.00 g, 4.4 mmol) and DIEA (1.14 g, 8.8 mmol). The reaction mixture was stirred at room temperature for 1 h and concentrated in vacuo. The residue was purified by a silica gel column, eluted with DCM and methanol, to afford 260 (3.18 g, 92% yield).

### Example 178. (8S, 11S, 14S)-14-((tert-butoxycarbonyl)amino)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11-isopropyl-8-methyl-4, 7, 10, 13-tetraoxo-3, 6, 9, 12-tetraazaheptadecan-17-oic acid (261)

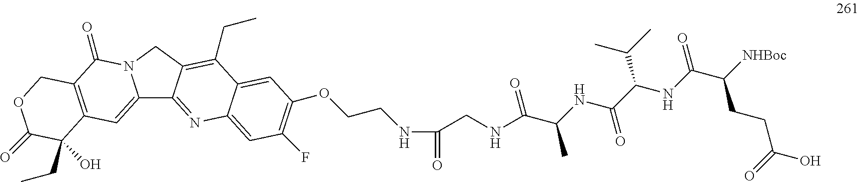 [SMILES: CCc1c2c(nc3cc(F)c(OCCNC(=O)CNC(=O)[C@H](C)NC(=O)[C@@H](NC(=O)[C@H](CCC(=O)O)NC(=O)OC(C)(C)C)C(C)C)cc13)-c1cc3c(c(=O)n1C2)COC(=O)[C@]3(O)CC]

To a solution of 260 (2.20 g, 2.333 mmol) in THF (50 mL) was added Pd/C (0.33 g, 10 wt %) under H₂ atmosphere at room temperature. The suspension was degassed and purged with H₂ for 3 times and stirred under H₂ at room temperature for 16 h, and filtered. The filtrate was concentrated under reduced pressure to give 261 (2.08 g, 2.439 mol, 104% yield) as a light-yellow foam. ESI-MS m/z: [M+H]⁺calcd. for C₄₄H₅₆FN₇O₁₃ 909.39; found 910.42.

### Example 179. tert-butyl (R)-4-((tert-butoxycarbonyl)amino)-5-(4-hydroxy-3-((5S, 8S)-5-isopropyl-8-methyl-3, 6, 9-trioxo-1-phenyl-2-oxa-4, 7, 10-triazadodecan-12-amido)phenyl) pentanoate (263)

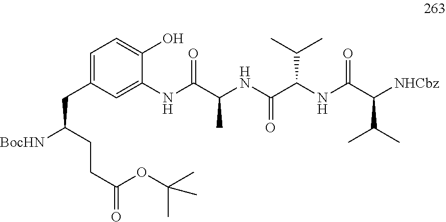 [SMILES: CC(C)[C@H](NC(=O)OCc1ccccc1)C(=O)N[C@H](C(=O)N[C@@H](C)C(=O)Nc1cc(C[C@@H](CCC(=O)OC(C)(C)C)NC(=O)OC(C)(C)C)ccc1O)C(C)C]

To a stirred solution of 27 (2.27 g, 5 mmol) in DCM (30 mL) at room temperature were added 262 (1.67 g, 5 mmol) and EDCI (1.49 g, 8 mmol). The reaction mixture was stirred at room temperature for 1 h, washed with water (100 mL), brine (100 mL), and concentrated in vacuo. The residue was purified by a silica gel column, eluted with ethyl acetate and petroleum ether, to afford 263 (3 g, 78% yield).

### Example 180. (R)-4-amino-5-(4-hydroxy-3-((5S, 8S)-5-isopropyl-8-methyl-3, 6, 9-trioxo-1-phenyl-2-oxa-4, 7, 10-triazadodecan-12-amido)phenyl) pentanoic acid (264)

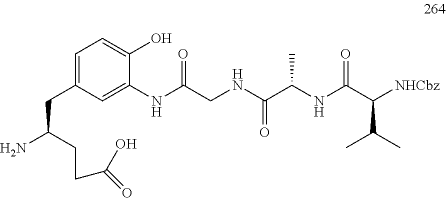 [SMILES: CC(C)[C@H](NC(=O)OCc1ccccc1)C(=O)N[C@@H](C)C(=O)NCC(=O)Nc1cc(C[C@H](N)CCC(=O)O)ccc1O]

To a stirred solution of 263 (3.00 g, 4 mmol) in DCM (40 mL) at room temperature was added TFA (30 mL). The reaction mixture was stirred at room temperature for 5 h, and then concentrated to afford 264 (1.5 g, 63% yield).

### Example 181. (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(4-hydroxy-3-((5S, 8S)-5-isopropyl-8-methyl-3, 6, 9-trioxo-1-phenyl-2-oxa-4, 7, 10-triazadodecan-12-amido)phenyl) pentanoic acid (265)

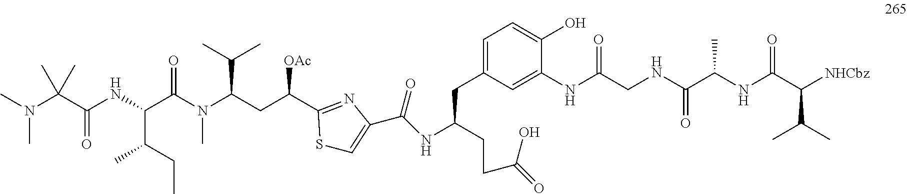 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)CNC(=O)[C@H](C)NC(=O)[C@@H](NC(=O)OCc3ccccc3)C(C)C)c2)cs1)C(C)C]

To a stirred solution of 264 (0.95 g, 1.6 mmol) in DMF (10 mL) at room temperature were added 24 (1.12 g, 1.6 mmol) and DIEA (0.52 g, 4 mmol). The reaction mixture was stirred at room temperature for 2 h, and then concentrated to afford 265 (1.78 g, 100% yield).

### Example 182. (R)-5-(3-(2-((S)-2-((S)-2-amino-3-methylbutanamido) propanamido) acetamido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (266)

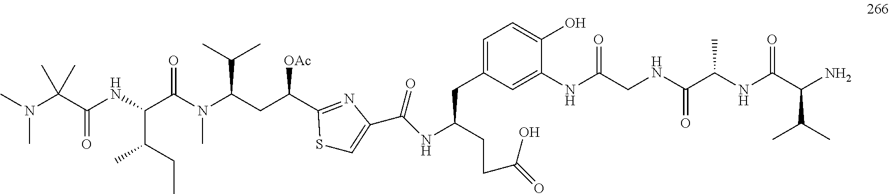 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)CNC(=O)[C@H](C)NC(=O)[C@@H](N)C(C)C)c2)cs1)C(C)C]

To a stirred solution of 265 (1.78 g, 1.6 mmol) in methanol (200 mL) at room temperature was added Pd/C (1.0 g, 10 wt %). The mixture was stirred overnight under a hydrogen balloon at room temperature, filtered over celite. The filtrate was concentrated to give 266 (600 mg, 38% yield).

### Example 183. (R)-5-(3-((8S, 11S, 14S, 19S, 22S)-14-((tert-butoxycarbonyl)amino)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11, 19-diisopropyl-8, 22-dimethyl-4, 7, 10, 13, 17, 20, 23-heptaoxo-3, 6, 9, 12, 18, 21, 24-heptaazahexacosan-26-amido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (267)

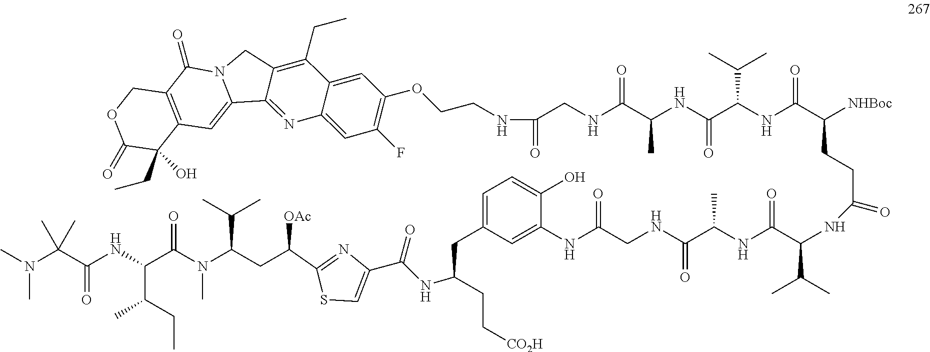 [SMILES: CCc1c2c(nc3cc(F)c(OCCNC(=O)CNC(=O)[C@H](C)NC(=O)[C@@H](NC(=O)[C@H](CCC(=O)N[C@H](C(=O)N[C@@H](C)C(=O)NCC(=O)Nc4cc(C[C@@H](CCC(=O)O)NC(=O)c5csc([C@@H](C[C@H](C(C)C)N(C)C(=O)[C@@H](NC(=O)C(C)(C)N(C)C)[C@@H](C)CC)OC(C)=O)n5)ccc4O)C(C)C)NC(=O)OC(C)(C)C)C(C)C)cc13)-c1cc3c(c(=O)n1C2)COC(=O)[C@]3(O)CC]

To a solution of compound 261 (110.0 mg, 0.109 mmol, 1.0 eq) and compound 266 (104.9 g, 0.109 mmol, 1.0 eq) in DMF (3 mL) was added DIEA (28.2 mg, 0.218 mmol, 2.0 eq). The mixture was stirred at room temperature for 1.5 h and quenched with HCOOH (0.5 mL), and then purified by flash column chromatography to give 267 (90.0 mg, 0.049 mmol, 44% yield) as a yellow solid. ESI-MS m/z: [M+H]+calcd. for C₉₀H₁₂₇FN₁₆O₂₃S 1852.15; found 1853.21.

### Example 184. (R)-5-(3-((8S, 11S, 14S, 19S, 22S)-14-amino-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11, 19-diisopropyl-8, 22-dimethyl-4, 7, 10, 13, 17, 20, 23-heptaoxo-3, 6, 9, 12, 18, 21, 24-heptaazahexacosan-26-amido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (268)

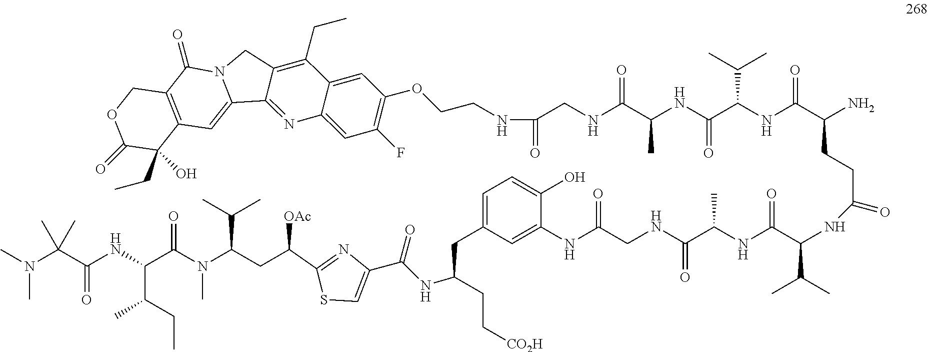 [SMILES: CCc1c2c(nc3cc(F)c(OCCNC(=O)CNC(=O)[C@H](C)NC(=O)[C@@H](NC(=O)[C@@H](N)CCC(=O)N[C@H](C(=O)N[C@@H](C)C(=O)NCC(=O)Nc4cc(C[C@@H](CCC(=O)O)NC(=O)c5csc([C@@H](C[C@H](C(C)C)N(C)C(=O)[C@@H](NC(=O)C(C)(C)N(C)C)[C@@H](C)CC)OC(C)=O)n5)ccc4O)C(C)C)C(C)C)cc13)-c1cc3c(c(=O)n1C2)COC(=O)[C@]3(O)CC]

To a solution of compound 267 (90.0 mg, 0.0486 mmol, 1.0 eq) in DCM (2 mL) was added TFA (0.5 mL). The mixture was stirred at room temperature for 2 h and concentrated under reduced pressure to give 268 (80.0 mg, 0.0457 mmol, 94% yield) as a light-yellow oil. ESI-MS m/z: [M+H]⁺ calcd. for Cs₅H₁₁₉FN₁₆O21S 1750.84; found 1752.01.

### Example 185. (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-1l-yl) thiazole-4-carboxamido)-5-(3-((13S, 21S, 26S, 29S)-21-(((S)-1-(((S)-1-((2-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-2-oxoethyl)amino)-1-oxopropan-2-yl)amino)-3-methyl-1-oxobutan-2-yl) carbamoyl)-13-(4-(2, 5-dioxo-2, 5-dihydro-1H-pyrrol-1-yl) butanamido)-26-isopropyl-29-methyl-7, 14, 19, 24, 27, 30-hexaoxo-2, 5-dioxa-8, 15, 20, 25, 28, 31-hexaazatritriacontan-33-amido)-4-hydroxyphenyl) pentanoic acid (269)

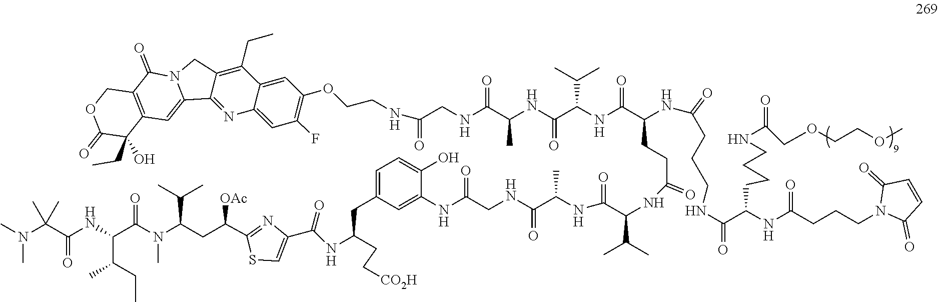

To a solution of compound 126 (29.6 mg, 0.0342 mmol, 1.0 eq) in DMF (3 mL) were added HATU (13.0 mg, 0.0342 mmol, 1.0 eq) and DIEA (8.85 mg, 0.0684 mmol, 2.0 eq). The mixture was stirred at room temperature for 10 min., and then compound 268 (60.0 mg, 0.0342 mmol, 1.0 eq) was added. After being stirred at room temperature for 0.5 h, the reaction was then quenched by HCOOH (0.5 mL), and purified by flash column chromatography to give 269 (31.6 mg, 0.0121 mmol, 35% yield) as a yellow solid. ESI-MS m/z: [M+H]⁺ calcd. for C₁₂₄H₁₈₅FN₂₀O₃₇S 2597.29; found 1299.71.

### Example 186. N-((benzyloxy) carbonyl)-N-(2-(((S)-1-(((S)-1-((2-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-2-oxoethyl)amino)-1-oxopropan-2-yl)amino)-3-methyl-1-oxobutan-2-yl)amino)-2-oxoethyl)glycine (271)

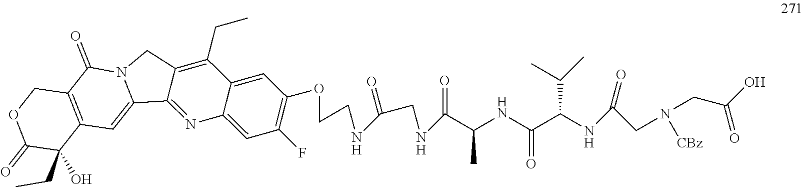

To a solution of 258 (417.4 mg, 1.675 mmol, 1.2 eq) in DMF (9 mL) was added benzyl 2, 6-dioxomorpholine-4-carboxylate (950.0 mg, 1.396 mmol, 1.0 eq). The mixture was stirred at room temperature for 1 h, and then quenched by water (0.5 mL), and concentrated. The residue was purified by flash column chromatography to give 271 (874 mg, 0.940 mmol, 67% yield) as a yellow solid. ESI-MS m/z: [M+H]⁺ calcd. for C₄₆H₅₂FN₇013 929.36; found 930.42.

### Example 187. (R)-5-(3-((5S, 8S, 16S, 19S)-12-((benzyloxy) carbonyl)-26-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-8, 16-diisopropyl-5, 19-dimethyl-4, 7, 10, 14, 17, 20, 23-heptaoxo-3, 6, 9, 12, 15, 18, 21, 24-octaazahexacosanamido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (272)

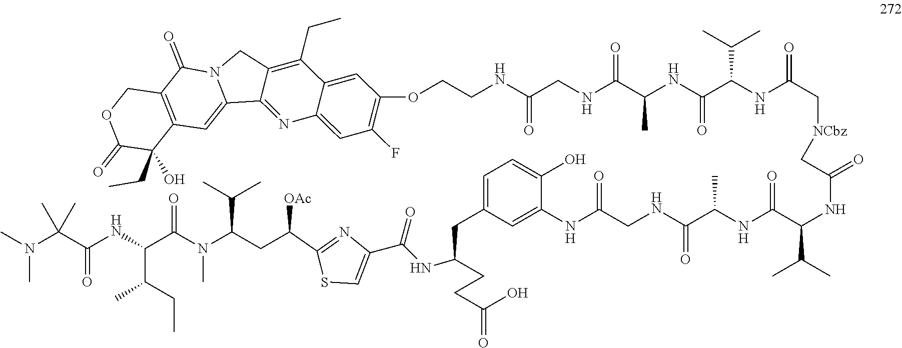 [SMILES: CCc1c2c(nc3cc(F)c(OCCNC(=O)CNC(=O)[C@H](C)NC(=O)[C@@H](NC(=O)CN(CC(=O)N[C@H](C(=O)N[C@@H](C)C(=O)NCC(=O)Nc4cc(C[C@@H](CCC(=O)O)NC(=O)c5csc([C@@H](C[C@H](C(C)C)N(C)C(=O)[C@@H](NC(=O)C(C)(C)N(C)C)[C@@H](C)CC)OC(C)=O)n5)ccc4O)C(C)C)C(=O)OCc4ccccc4)C(C)C)cc13)-c1cc3c(c(=O)n1C2)COC(=O)[C@]3(O)CC]

To a solution of 271 (100.0 mg, 0.108 mmol, 1.0 eq) and HATU (45.0 mg, 0.118 mmol, 1.1 eq) in DMF (3 mL) was added DIEA (34.8 mg, 0.269 mmol, 2.5 eq). The mixture was stirred at room temperature for 10 min. and 266 (103.4 mg, 0.108 mmol, 1.0 eq) was added. After being stirred at room temperature for 0.5 h, the reaction was quenched by HCOOH (0.5 mL), then purified by prep-HPLC to give 272 (90.0 mg, 0.0481 mmol, 45% yield) as a white solid. ESI-MS m/z: [M+H]⁺calcd. for C₉₂ H₁₂₃FN₁₆O₂₃S 1870.87; found 1872.01.

### Example 188. (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((5S, 8S, 16S, 19S)-26-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-8, 16-diisopropyl-5, 19-dimethyl-4, 7, 10, 14, 17, 20, 23-heptaoxo-3, 6, 9, 12, 15, 18, 21, 24-octaazahexacosanamido)-4-hydroxyphenyl) pentanoic acid (273)

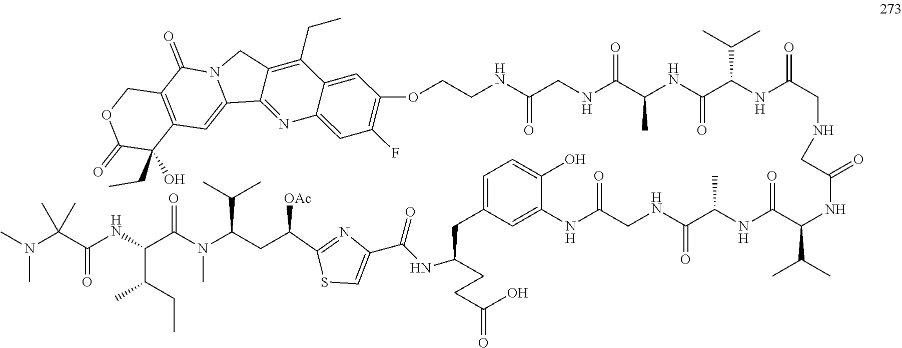 [SMILES: CCc1c2c(nc3cc(F)c(OCCNC(=O)CNC(=O)[C@H](C)NC(=O)[C@@H](NC(=O)CNCC(=O)N[C@H](C(=O)N[C@@H](C)C(=O)NCC(=O)Nc4cc(C[C@@H](CCC(=O)O)NC(=O)c5csc([C@@H](C[C@H](C(C)C)N(C)C(=O)[C@@H](NC(=O)C(C)(C)N(C)C)[C@@H](C)CC)OC(C)=O)n5)ccc4O)C(C)C)C(C)C)cc13)-c1cc3c(c(=O)n1C2)COC(=O)[C@]3(O)CC]

To a solution of 272 (90.0 mg, 0.0481 mmol, 1.0 eq) in methanol (20 mL) at room temperature was added Pd/C (0.1 g, 10 wt %). The mixture was stirred overnight under a hydrogen balloon at room temperature, and filtered over celite. The filtrate was concentrated to give 273 (35 mg, 0.0201 mmol, 42% yield) as a yellow solid. ESI-MS m/z: [M+H]⁺ calcd. for C₈₄H₁₁₇FN₁₆O₂₁S 1736.83; found 1737.92.

### Example 189. (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((13S, 24S, 27S)-20-((4S, 7S)-14-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-4-isopropyl-7-methyl-2, 5, 8, 11-tetraoxo-3, 6, 9, 12-tetraazatetradecyl)-13-(4-(2, 5-dioxo-2, 5-dihydro-IH-pyrrol-1-yl) butanamido)-24-isopropyl-27-methyl-7, 14, 19, 22, 25, 28-hexaoxo-2, 5-dioxa-8, 15, 20, 23, 26, 29-hexaazahentriacontan-31-amido)-4-hydroxyphenyl) pentanoic acid (274)

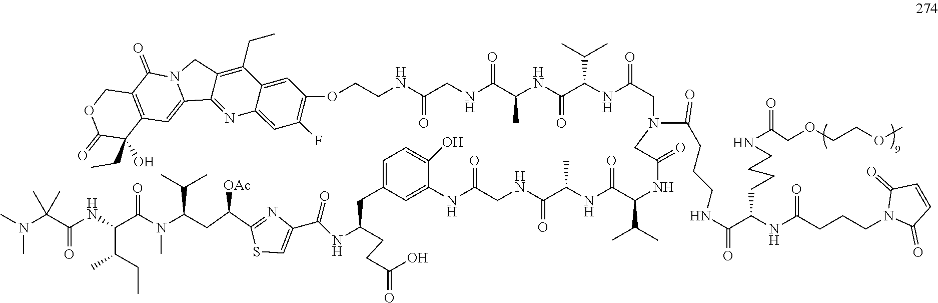

To a solution of 126 (17.4 mg, 0.0201 mmol, 1.0 eq) in DMF (1 mL) were added HATU (8.0 mg, 0.0211 mmol, 1.05 eq) and DIEA (5.2 mg, 0.0403 mmol, 2.0 eq). The mixture was stirred at room temperature for 10 min. and then 273 (35.0 mg, 0.0201 mmol, 1.0 eq) was added. After being stirred at room temperature for 1 h, the reaction was quenched by HCOOH (0.5 mL), then purified by flash column chromatography to give 274 (28.8 mg, 0.0111 mmol, 57% yield) as a white solid. ESI-MS m/z: [M+H]+calcd. for C₁₂₃H₁₈₃FN₂₀O₃₇S 2583.28; found 1292.75.

### Example 190. benzyl (S)-4-((tert-butoxycarbonyl)amino)-5-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl)amino)-5-oxopentanoate (276)

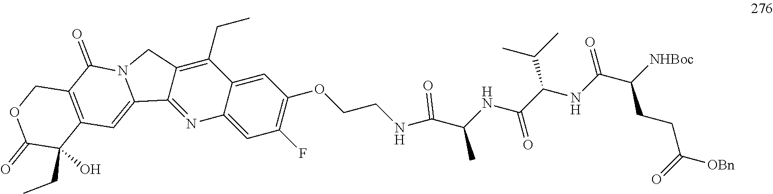 [SMILES: CCc1c2c(nc3cc(F)c(OCCNC(=O)[C@H](C)NC(=O)[C@@H](NC(=O)[C@H](CCC(=O)OCc4ccccc4)NC(=O)OC(C)(C)C)C(C)C)cc13)-c1cc3c(c(=O)n1C2)COC(=O)[C@]3(O)CC]

To a solution of 131 (1.06 g, 1.700 mmol, 1.0 eq) in DMF (15 mL) was added HATU (0.78 g, 2.040 mmol, 1.2 eq). The mixture was stirred at room temperature for 5 min. and then 259 (0.63 g, 1.870 mmol, 1.1 eq) and DIEA (0.44 g, 3.399 mmol, 2.0 eq) were added. The reaction mixture was stirred at room temperature for 0.5 h and concentrated under reduced pressure. The residue was recrystallized from ethyl acetate/petroleum ether (1:1) to give 276 (2.20 g, 2.333 mmol, 137% yield) as a yellow solid. ESI-MS m/z: [M+H]⁺ calcd. for C₄₉H₅₉FN₆O₁₂ 942.42; found 943.56.

### Example 191. (S)-4-((tert-butoxycarbonyl)amino)-5-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl)amino)-5-oxopentanoic acid (277)

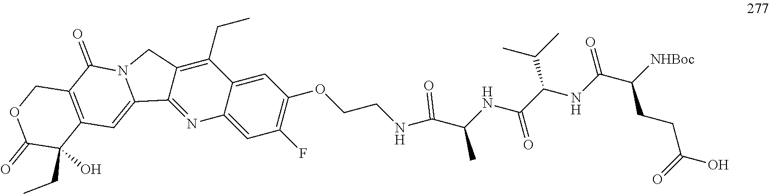 [SMILES: CCc1c2c(nc3cc(F)c(OCCNC(=O)[C@H](C)NC(=O)[C@@H](NC(=O)[C@H](CCC(=O)O)NC(=O)OC(C)(C)C)C(C)C)cc13)-c1cc3c(c(=O)n1C2)COC(=O)[C@]3(O)CC]

To a solution of compound 276 (2.20 g, 2.333 mmol) in THF (50 mL) was added Pd/C (0.33 g, 10 wt %) under H₂ atmosphere at room temperature. The suspension was degassed and purged with H₂ for 3 times, and then stirred under H₂ at room temperature for 16 h. The reaction mixture was filtered and the filtrate was concentrated under reduced pressure to give compound 277 (2.08 g, 2.439 mol, 104% yield) as a light-yellow foam. ESI-MS m/z: [M+H]⁺ calcd. for C₄₄H₅₆FN₇O₁₃ 909.39; found 910.42.

### Example 192. tert-butyl (R)-5-(3-((S)-2-((S)-2-(((benzyloxy) carbonyl)amino)-3-methylbutanamido) propanamido)-4-hydroxyphenyl)-4-((tert-butoxycarbonyl)amino) pentanoate (278)

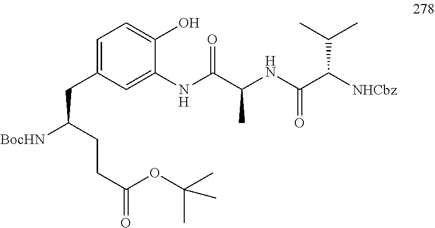 [SMILES: CC(C)[C@H](NC(=O)OCc1ccccc1)C(=O)N[C@@H](C)C(=O)Nc1cc(C[C@@H](CCC(=O)OC(C)(C)C)NC(=O)OC(C)(C)C)ccc1O]

To a stirred solution of 21 (7.7 g, 17.052 mmol) in DCM (100 mL) at room temperature were added 262 (4.28 g, 17.052 mmol) and EDCI (6.54 g, 34.104 mmol). The reaction mixture was stirred at room temperature for 1 h, washed with water (100 mL), brine (100 mL), and concentrated in vacuo. The residue was purified by a silica gel column, eluted with ethyl acetate and petroleum ether, to afford 278 (5.54 g, 47% yield).

### Example 193. (R)-4-amino-5-(3-((S)-2-((S)-2-(((benzyloxy) carbonyl)amino)-3-methylbutanamido) propanamido)-4-hydroxyphenyl) pentanoic acid (279)

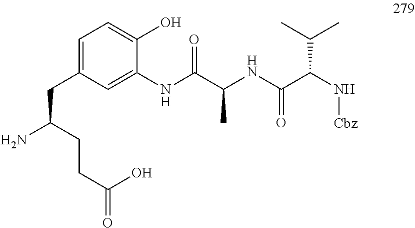 [SMILES: CC(C)[C@H](NC(=O)OCc1ccccc1)C(=O)N[C@@H](C)C(=O)Nc1cc(C[C@H](N)CCC(=O)O)ccc1O]

To a solution of compound 278 (1 g, 1.4 mmol) in DCM (10 mL) was added TFA (2 mL). The mixture was stirred at room temperature for 4 h and concentrated under reduced pressure to give 279 (0.7 g, 91% yield).

### Example 194. (R)-5-(3-((S)-2-((S)-2-(((benzyloxy) carbonyl)amino)-3-methylbutanamido) propanamido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (280)

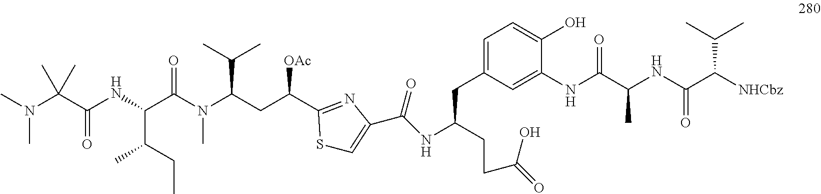 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)[C@H](C)NC(=O)[C@@H](NC(=O)OCc3ccccc3)C(C)C)c2)cs1)C(C)C]

To a stirred solution of 279 (0.468 g, 0.89 mmol) in DMF (10 mL) at room temperature were added 24 (0.735 g, 1.1 mmol) and DIEA (286 mg, 2.2 mmol). The reaction mixture was stirred at room temperature for 0.5 h, and then purified by prep-HPLC, lyophilized to afford 280 (0.935 g, 102% yield).

### Example 195. (R)-5-(3-((S)-2-((S)-2-amino-3-methylbutanamido) propanamido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (281)

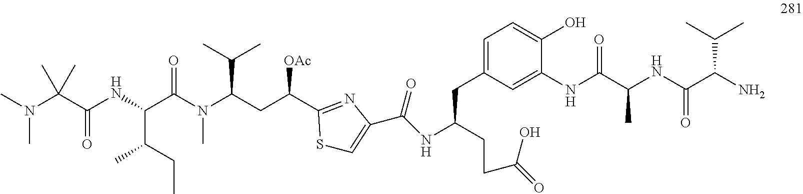 [SMILES: CC[C@H](C)[C@H](NC(=O)C(C)(C)N(C)C)C(=O)N(C)[C@H](C[C@@H](OC(C)=O)c1nc(C(=O)N[C@H](CCC(=O)O)Cc2ccc(O)c(NC(=O)[C@H](C)NC(=O)[C@@H](N)C(C)C)c2)cs1)C(C)C]

To a stirred solution of 280 (749 mg, 0.7 mmol) in methanol (200 mL) at room temperature was added Pd/C (1.0 g, 10 wt %). The mixture was stirred overnight under a hydrogen balloon at room temperature, and filtered over celite. The filtrate was concentrated, purified by prep-HPLC, lyophilized to afford 281 (452 mg, 69% yield).

### Example 196. (R)-5-(3-((5S, 8S, 11S, 16S, 19S)-11-((tert-butoxycarbonyl)amino)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-8, 16-diisopropyl-5, 19-dimethyl-4, 7, 10, 14, 17-pentaoxo-3, 6, 9, 15, 18-pentaazaicosan-20-amido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (282)

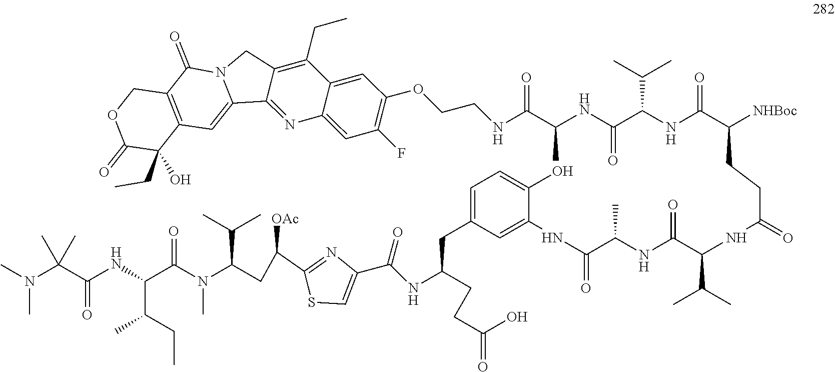 [SMILES: CCc1c2c(nc3cc(F)c(OCCNC(=O)[C@H](C)NC(=O)[C@@H](NC(=O)[C@H](CCC(=O)N[C@H](C(=O)N[C@@H](C)C(=O)Nc4cc(C[C@@H](CCC(=O)O)NC(=O)c5csc([C@@H](C[C@H](C(C)C)N(C)C(=O)[C@@H](NC(=O)C(C)(C)N(C)C)[C@@H](C)CC)OC(C)=O)n5)ccc4O)C(C)C)NC(=O)OC(C)(C)C)C(C)C)cc13)-c1cc3c(c(=O)n1C2)COC(=O)[C@]3(O)CC]

To a solution of 277 (153.0 mg, 0.179 mmol, 1.0 eq) in DMF (5 mL) were added HATU (71.6 mg, 0.188 mmol, 1.05 eq) and DIEA (34.8 mg, 0.269 mmol, 1.5 eq). The mixture was stirred at room temperature for 10 min., and then 281 (162.0 mg, 0.179 mmol, 1.0 eq) was added. The reaction mixture was stirred at room temperature for 0.5 h, quenched by HCOOH (0.5 mL), and then purified by flash column chromatography to give 282 (75.0 mg, 0.0432 mmol, 24% yield) as a yellow solid. ESI-MS m/z: [M+H]⁺ calcd. for C₈₆H₁₂₁FN₁₄O₂₁S 1736.85; found 1737.90.

### Example 197. (R)-5-(3-((5S, 8S, 11S, 16S, 19S)-11-amino-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-8, 16-diisopropyl-5, 19-dimethyl-4, 7, 10, 14, 17-pentaoxo-3, 6, 9, 15, 18-pentaazaicosan-20-amido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (283)

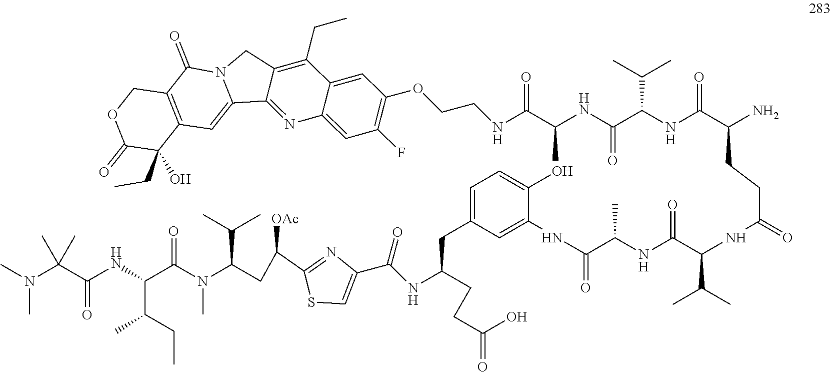 [SMILES: CCc1c2c(nc3cc(F)c(OCCNC(=O)[C@H](C)NC(=O)[C@@H](NC(=O)[C@@H](N)CCC(=O)N[C@H](C(=O)N[C@@H](C)C(=O)Nc4cc(C[C@@H](CCC(=O)O)NC(=O)c5csc([C@@H](C[C@H](C(C)C)N(C)C(=O)[C@@H](NC(=O)C(C)(C)N(C)C)[C@@H](C)CC)OC(C)=O)n5)ccc4O)C(C)C)C(C)C)cc13)-c1cc3c(c(=O)n1C2)COC(=O)[C@]3(O)CC]

To a solution of 282 (75.0 mg, 0.0432 mmol, 1.0 eq) in DCM (2 mL) was added TFA (0.5 mL). The mixture was stirred at room temperature for 1 h and concentrated under reduced pressure to give 283 (71.0 mg, 0.0433 mmol, 100% yield) as a light-yellow oil. ESI-MS m/z: [M+H]⁺calcd. for C₈₁H₁₁₃FN₁₄O₁₉S 1636.80; found 1636.92.

### Example 198. (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((13S, 21S, 26S, 29S)-21-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl) carbamoyl)-13-(4-(2, 5-dioxo-2, 5-dihydro-1H-pyrrol-1-yl) butanamido)-26-isopropyl-29-methyl-7, 14, 19, 24, 27-pentaoxo-2, 5-dioxa-8, 15, 20, 25, 28-pentaazatriacontan-30-amido)-4-hydroxyphenyl) pentanoic acid (284)

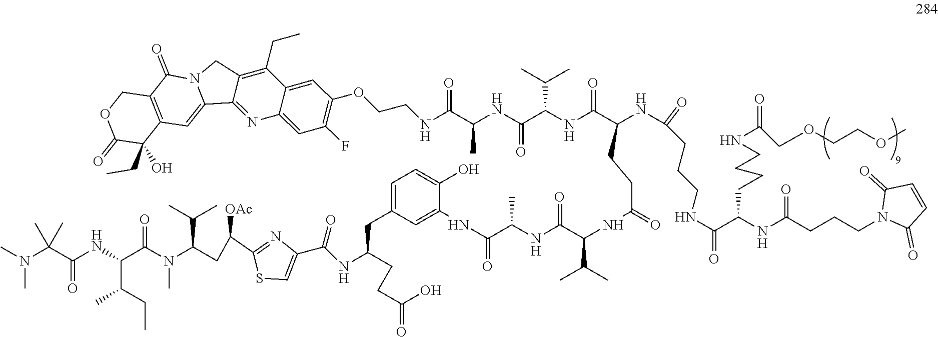

To a solution of 126 (37.5 mg, 0.0433 mmol, 1.0 eq) in DMF (3 mL) were added HATU (17.3 mg, 0.0455 mmol, 1.05 eq) and DIEA (11.2 mg, 0.0867 mmol, 2.0 eq). The mixture was stirred at room temperature for 10 min. and then 283 (71 mg, 0.0433 mmol, 1.0 eq) was added. The reaction mixture was stirred at room temperature for 0.5 h, quenched by HCOOH (0.5 mL), and then purified by flash column chromatography to give 284 (23.0 mg, 0.00926 mmol, 210% yield) as a white solid. ESI-MS m/z: [M+H]+calcd. for C₁₂₀H₁₇₉FN₁₈O₃₅S 2483.25; found 1242.63.

### Example 199. tert-butyl (R)-5-(3-((S)-2-((S)-2-amino-3-methylbutanamido) propanamido)-4-hydroxyphenyl)-4-((tert-butoxycarbonyl)amino) pentanoate (286)

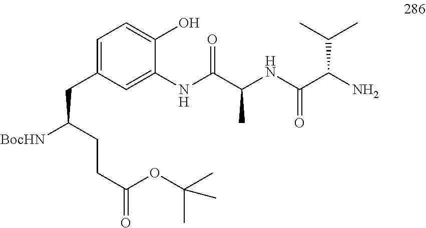 [SMILES: CC(C)[C@H](N)C(=O)N[C@@H](C)C(=O)Nc1cc(C[C@@H](CCC(=O)OC(C)(C)C)NC(=O)OC(C)(C)C)ccc1O]

To a stirred solution of 278 (500 mg, 0.7 mmol) in methanol (10 mL) at room temperature was added Pd/C (0.1 g, 10 wt %). The mixture was stirred overnight under a hydrogen balloon at room temperature and filtered over celite. The filtrate was concentrated, purified by prep-HPLC, lyophilized to afford 286 (380 mg, 95% yield).

### Example 200. tert-butyl (R)-4-((tert-butoxycarbonyl)amino)-5-(3-((13S, 21S, 26S, 29S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-26-isopropyl-29-methyl-7, 14, 19, 24, 27-pentaoxo-2, 5-dioxa-8, 15, 20, 25, 28-pentaazatriacontan-30-amido)-4-hydroxyphenyl) pentanoate (287)

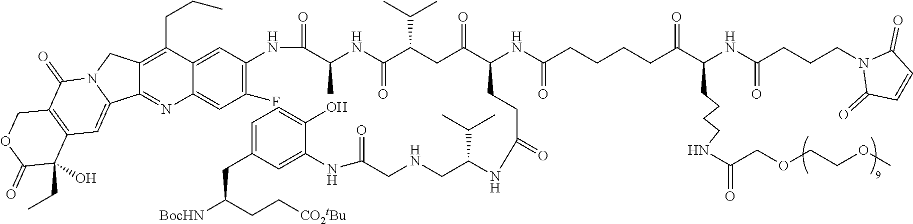

To a solution of 140 (389.9 mg, 0.251 mmol, 1.0 eq) in DMF (15 mL) was added HATU (105.1 mg, 0.276 mmol, 1.1 eq). The mixture was stirred at room temperature for 5 min. and then 286 (166.1 mg, 0.302 mmol, 1.2 eq) and DIEA (65.0 mg, 0.503 mmol, 2.0 eq) were added. The reaction mixture was stirred at room temperature for 0.5 h and concentrated under reduced pressure. The residue was purified by prep-HPLC to give 287 (210.0 mg, 0.101 mmol, 40% yield) as a yellow solid. ESI-MS m/z: [M+H]⁺ calcd. for C₁₀₃H₁₅₄N₁₄O₃₁ 2083.09; found 2084.15.

### Example 201. (R)-4-amino-5-(3-((13S, 21S, 26S, 29S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-26-isopropyl-29-methyl-7, 14, 19, 24, 27-pentaoxo-2, 5-dioxa-8, 15, 20, 25, 28-pentaazatriacontan-30-amido)-4-hydroxyphenyl) pentanoic acid (288)

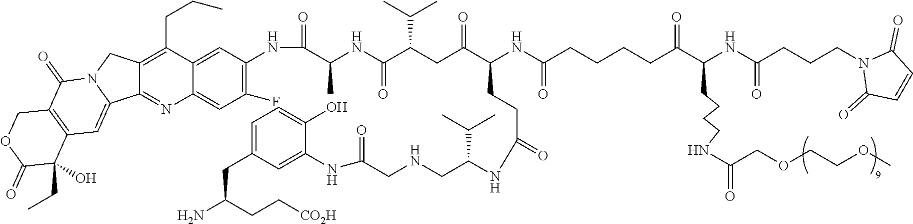

To a solution of 287 (210.0 mg, 0.101 mmol) in DCM (6 mL) was added TFA (4 mL). The mixture was stirred at room temperature for 1 h and concentrated under reduced pressure. The residue was washed with MTBE and dried under vacuum to give 288 (194.3 mg, 0.101 mmol, 100% yield) as a yellow solid. ESI-MS m/z: [M+H]⁺calcd. for C₉₄H₁₃₈N₁₄O₂₉ 1926.98; found 1928.02.

### Example 202. (R)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido)-5-(3-((13S, 21S, 26S, 29S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-26-isopropyl-29-methyl-7, 14, 19, 24, 27-pentaoxo-2, 5-dioxa-8, 15, 20, 25, 28-pentaazatriacontan-30-amido)-4-hydroxyphenyl) pentanoic acid (289)

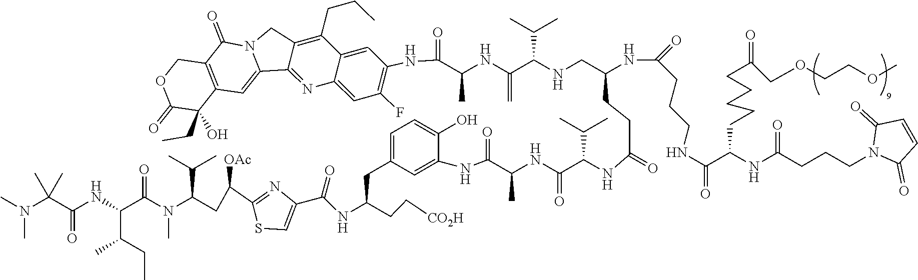

To a solution of 288 (195.0 mg, 0.101 mmol, 1.0 eq) and 24 (84.1 mg, 0.121 mmol, 1.2 eq) in DMF (3 mL) was added DIEA (26.2 mg, 0.202 mmol, 2.0 eq). The reaction mixture was stirred at room temperature for 0.5 h and quenched by HCOOH (0.5 mL), purified by flash column chromatography to give 289 (157.0 mg, 0.064 mmol, 64% yield) as a light yellow solid. ESI-MS m/z: [M+H]⁺calcd. for C₁₁₉H₁₇₈N₁₈O₃₄S 2435.25; found 2436.51.

### Example 203. Synthesis of tert-butyl 2-((2S, 5R, 8S, 11S)-5-benzyl-8-((13S, 21S)-13-(4-(2, 5-dioxo-2, 5-dihydro-IH-pyrrol-1-yl) butanamido)-21-(((S)-1-(((S)-1-(((S)-4-ethyl-4-hydroxy-3, 14-dioxo-1 1-propyl-3, 4, 12, 14-tetrahydro-1H-pyrano[3′, 4′:6, 7]indolizino [1,2-b]quinolin-9-yl)amino)-1-oxopropan-2-yl)amino)-3-methyl-1-oxobutan-2-yl) carbamoyl)-7, 14, 19, 24-tetraoxo-2, 5-dioxa-8, 15, 20, 25-tetraazanonacosan-29-yl)-11-(3- guanidinopropyl)-3, 6, 9, 12, 15-pentaoxo-1, 4, 7, 10, 13-pentaazacyclopentadecan-2-yl)acetate tertbutyl 2-((2S, 5R, 8S, 11S)-5-benzyl-8-((13S, 21S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-7, 14, 19, 24-tetraoxo-2, 5-dioxa-8, 15, 20, 25-tetraazanonacosan-29-yl)-11-(3- guanidinopropyl)-3, 6, 9, 12, 15-pentaoxo-1, 4, 7, 10, 13-pentaazacyclopentadecan-2-yl)acetate (292)

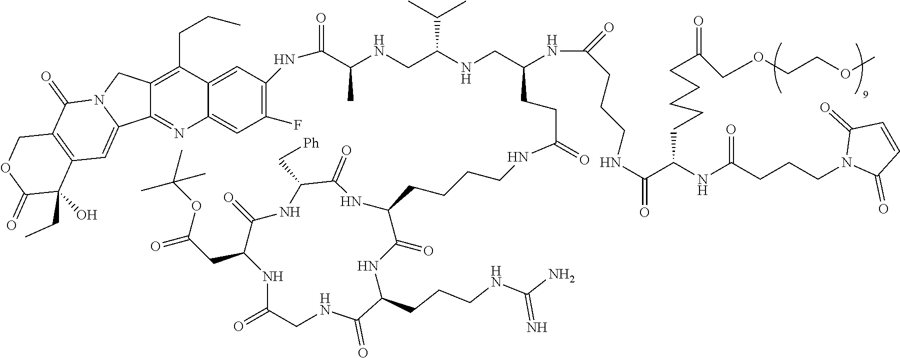

To a stirred solution of 140 (44 mg, 0.032 mmol) in DCM (5 mL) were added NHS (5 mg, 0.048 mmol) and EDCI (25 mg, 0.129 mmol) at room temperature. The reaction mixture was stirred at room temperature for 4 h, quenched with water (10 mL) and extracted with DCM (10 mL×2). The organic layers were combined and washed with brine (10 mL×2), dried over anhydrous sodium sulfate and concentrated in vacuo to afford an intermediate. To the intermediate in DMF (2 mL) were added 291 (32 mg, 0.048 mmol) and Na₂HPO₄/NaH₂PO₄ buffer solution (0.2 mol/L, pH=6.3) (0.32 mL, 0.064 mmol, 2.0 eq). The mixture was stirred at room temperature for 3 h and concentrated in vacuo to afford the crude product of 292 (58 mg, 100% yield).

### Example 204. Synthesis of 2-((2S, 5R, 8S, 11S)-5-benzyl-8-((13S, 21S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-IH-pyrano [3′, 4′:6, 7]indolizino [1,2-b]quinolin-9-yl)amino)-1-oxopropan-2-yl)amino)-3-methyl-1-oxobutan-2-yl) carbamoyl)-7, 14, 19, 24-tetraoxo-2, 5-dioxa-8, 15, 20, 25-tetraazanonacosan-29-yl)-11-(3- guanidinopropyl)-3, 6, 9, 12, 15-pentaoxo-1, 4, 7, 10, 13-pentaazacyclopentadecan-2-yl) acetic acid (293)

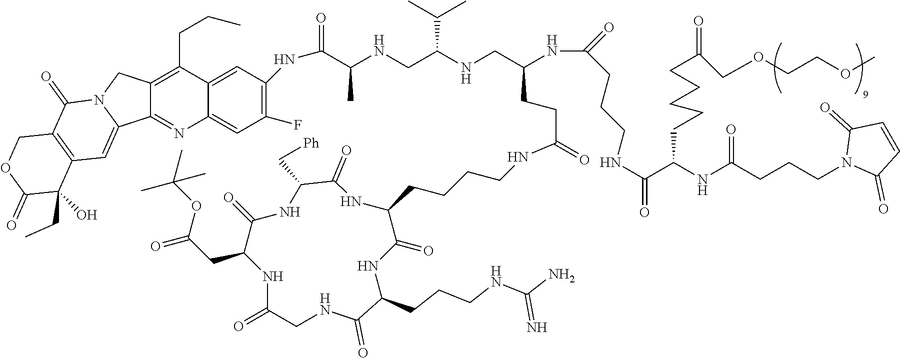

To a stirred solution of 292 (58 mg, 0.027 mmol) in DCM (1 mL) at room temperature was added TFA (0.5 mL). The reaction mixture was stirred at room temperature for 0.5 h, concentrated and purified by prep-HPLC, lyophilized to afford 293 (10 mg, 17% yield) as a white solid.

### Example 205. Synthesis of ((2S)-2-(2-((S)-1-(N2-N6-(tert-butoxycarbonyl)-N2-((13S, 21S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-7, 14, 19-trioxo-2, 5-dioxa-8, 15, 20-triazatetracosan-24-oyl)-L-lysyl-L-prolyl-N2-methyl-L-arginyl-L-arginyl) pyrrolidine-2-carboxamido)-3-(4-hydroxy-2, 6-dimethylphenyl) propanamido)-3, 3-dimethylbutanoyl)-L-leucine (296)

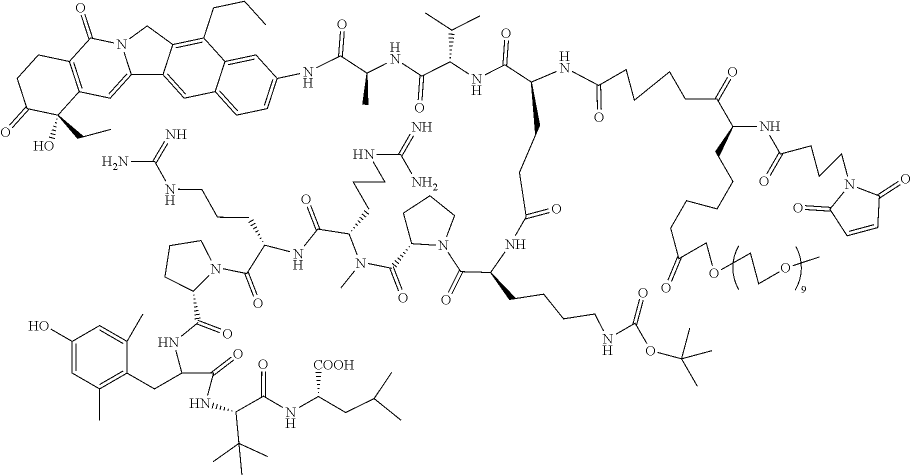

To a stirred solution of 140 (44 mg, 0.032 mmol) in DCM (5 mL) were added NHS (5 mg, 0.048 mmol) and EDCI (25 mg, 0.129 mmol) at room temperature. The reaction mixture was stirred at room temperature for 4 h, and extracted with DCM (10 mL×2) and water (10 mL). The organic layers were combined and washed with brine (10 mL×2), dried over anhydrous sodium sulfate and concentrated in vacuo to afford an intermediate compound. To the crude in DMF (2 mL) were added 295 (32 mg, 0.048 mmol) and Na₂HPO₄/NaH₂PO₄ buffer solution (0.2 mol/L, pH=6.3) (0.32 mL, 0.064 mmol, 2.0 eq). The mixture was stirred at room temperature for 3 h, and then concentrated in vacuo to afford 296 (73 mg, 100% yield).

### Example 206. Synthesis of ((2S)-2-(2-((S)-1-(N2-((13S, 21S)-13-(4-(2, 5-dioxo-2, 5-dihydro-1H-pyrrol-1-yl) butanamido)-21-(((S)-1-(((S)-1-(((S)-4-ethyl-4-hydroxy-3, 14-dioxo-11-propyl-3, 4, 12, 14-tetrahydro-1H-pyrano[3′, 4′:6, 7]indolizino[1, 2-b]quinolin-9-yl)amino)-1-oxopropan-2-yl)amino)-3-methyl-1-oxobutan-2-yl) carbamoyl)-7, 14, 19-trioxo-2, 5-dioxa-8, 15, 20-triazatetracosan-24-oyl)-L-lysyl-L-prolyl-N2-methyl-L-arginyl-L-arginyl) pyrrolidine-2-carboxamido)-3-(4-hydroxy-2, 6-dimethylphenyl) propanamido)-3, 3-dimethylbutanoyl)-L-leucine (297)

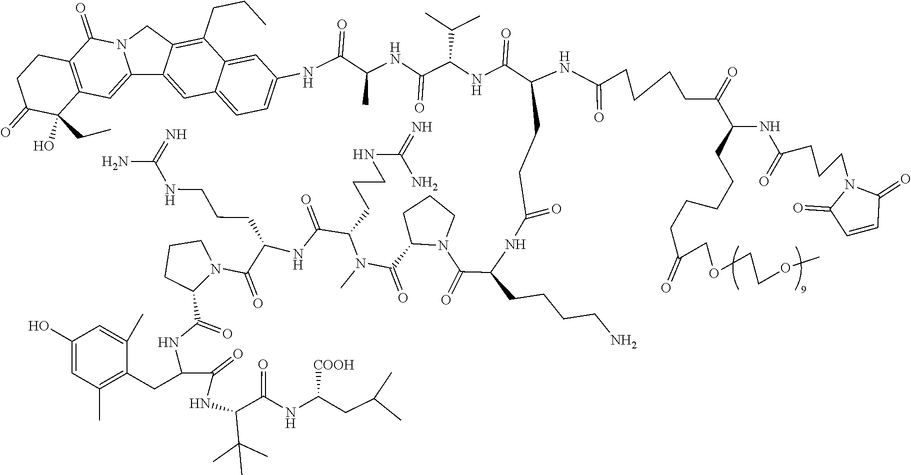

To a stirred solution of 296 (73 mg, 0.027 mmol) in DCM (1 mL) at room temperature was added TFA (0.5 mL). The reaction mixture was stirred at room temperature for 0.5 h, purified by prep-HPLC, lyophilized to afford 297 (10 mg, 17% yield) as a white solid.

### Example 207. Synthesis of benzyl (R)-4-(((benzyloxy) carbonyl)amino)-5-(((S)-1-(((S)-1-((5-((R)-5-(tert-butoxy)-2-((tert-butoxycarbonyl)amino)-5-oxopentyl)-2-hydroxyphenyl)amino)-1-oxopropan-2-yl)amino)-3-methyl-1-oxobutan-2-yl)amino)-5-oxopentanoate (299)

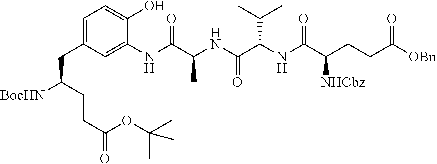 [SMILES: CC(C)[C@H](NC(=O)[C@@H](CCC(=O)OCc1ccccc1)NC(=O)OCc1ccccc1)C(=O)N[C@@H](C)C(=O)Nc1cc(C[C@@H](CCC(=O)OC(C)(C)C)NC(=O)OC(C)(C)C)ccc1O]

To a stirred solution of 286 (1.80 g, 3.26 mmol) in DCM (30 mL) at room temperature were added 298 (1.21 g, 3.26 mmol), HATU (1.24 g, 3.26 mmol) and DIEA (0.63 g, 4.90 mmol), The reaction mixture was stirred at room temperature for 1 h, diluted with DCM (50 mL), washed with water (50 mL), brine (50 mL), concentrated. The residue was purified by a silica gel column, eluted with petroleum ether and ethyl acetate, to afford 299 (2.40 g, 81% yield) as a white solid.

### Example 208. (R)-4-amino-5-(((S)-1-(((S)-1-((5-((R)-5-(tert-butoxy)-2-((tert-butoxycarbonyl)amino)-5-oxopentyl)-2-hydroxyphenyl)amino)-1-oxopropan-2-yl)amino)-3-methyl-1-oxobutan-2-yl)amino)-5-oxopentanoic acid (300)

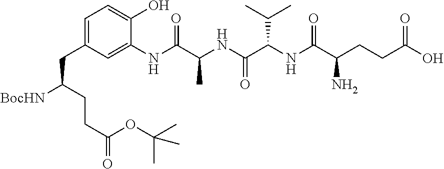 [SMILES: CC(C)[C@H](NC(=O)[C@H](N)CCC(=O)O)C(=O)N[C@@H](C)C(=O)Nc1cc(C[C@@H](CCC(=O)OC(C)(C)C)NC(=O)OC(C)(C)C)ccc1O]

To a stirred solution of 299 (2.40 g, 2.65 mmol) in isopropyl (120 mL) at room temperature was added Pd/C (1.0 g, 10 wt %). The reaction flask was evacuated and back filled with hydrogen for three times, and the mixture was stirred overnight under a hydrogen balloon at room temperature, filtered, concentrated to afford 300 (1.80 g, 100% yield) as a brown solid.

### Example 209. (R)-5-(((S)-1-(((S)-1-((5-((R)-5-(tert-butoxy)-2-((tert-butoxycarbonyl)amino)-5-oxopentyl)-2-hydroxyphenyl)amino)-1-oxopropan-2-yl)amino)-3-methyl-1-oxobutan-2-yl)amino)-4-(4-(2, 5-dioxo-2, 5-dihydro-1H-pyrrol-1-yl) butanamido)-5-oxopentanoic acid (301)

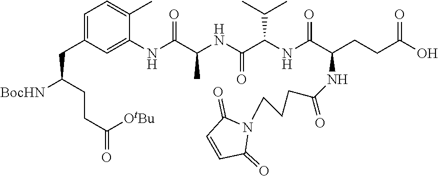 [SMILES: Cc1ccc(C[C@@H](CCC(=O)OC(C)(C)C)NC(=O)OC(C)(C)C)cc1NC(=O)[C@H](C)NC(=O)[C@@H](NC(=O)[C@@H](CCC(=O)O)NC(=O)CCCN1C(=O)C=CC1=O)C(C)C]

To a stirred solution of 300 (1.80 g, 2.65 mmol) in DMF (10 mL) at room temperature were added 19 (0.74 g, 2.64 mmol) and DIEA (0.51 g, 3.97 mmol). The reaction mixture was stirred at room temperature for 2 h, diluted with DCM (100 mL), washed with water (50 mL×3), brine (50 mL), concentrated. The residue was purified by a silica gel column, eluted with DCM and ethyl methanol, to afford 301 (569 mg, 25% yield) as a light-yellow liquid.

### Example 210. Synthesis of tert-butyl (R)-5-(3-((S)-2-((S)-2-((R)-5-((4-((2S, 5S, 11S, 14R)-14-benzyl-11-(2-(tert-butoxy)-2-oxoethyl)-5-(3- guanidinopropyl)-3, 6, 9, 12, 15-pentaoxo-1, 4, 7, 10, 13-pentaazacyclopentadecan-2-yl)butyl)amino)-2-(4-(2, 5-dioxo-2, 5-dihydro-1H-pyrrol-1-yl) butanamido)-5-oxopentanamido)-3-methylbutanamido) propanamido)-4-hydroxyphenyl)-4-((tert-butoxycarbonyl)amino) pentanoate (302)

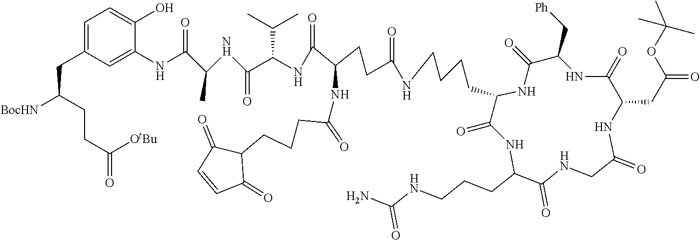 [SMILES: CC(C)[C@H](NC(=O)[C@@H](CCC(=O)NCCCC[C@@H]1NC(=O)[C@@H](Cc2ccccc2)NC(=O)[C@H](CC(=O)OC(C)(C)C)NC(=O)CNC(=O)C(CCCNC(N)=O)NC1=O)NC(=O)CCCC1C(=O)C=CC1=O)C(=O)N[C@@H](C)C(=O)Nc1cc(C[C@@H](CCC(=O)OC(C)(C)C)NC(=O)OC(C)(C)C)ccc1O]

To a stirred solution of 301 (232 mg, 0.27 mmol) in DMF (10 mL) at room temperature were added 291 (230.0 mg, 0.34 mmol), HATU (126.0 mg, 0.33 mmol) and DIEA (54 mg, 0.41 mmol). The reaction mixture was stirred at room temperature for 0.5 h. Purification by prep-HPLC and lyophilization afforded 302 (71 mg, 17% yield) as a light-yellow liquid.

### Example 211. Synthesis of (R)-4-amino-5-(3-((S)-2-((S)-2-((R)-5-((4-((2S, 5S, 11S, 14R)-14-benzyl-11-(carboxymethyl)-5-(3- guanidinopropyl)-3, 6, 9, 12, 15-pentaoxo-1, 4, 7, 10, 13-pentaazacyclopentadecan-2-yl)butyl)amino)-2-(4-(2, 5-dioxo-2, 5-dihydro-1H-pyrrol-1-yl) butanamido)-5-oxopentanamido)-3-methylbutanamido) propanamido)-4-hydroxyphenyl) pentanoic acid (303)

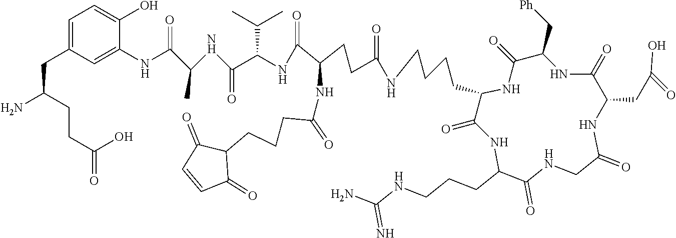 [SMILES: CC(C)[C@H](NC(=O)[C@@H](CCC(=O)NCCCC[C@@H]1NC(=O)[C@@H](Cc2ccccc2)NC(=O)[C@H](CC(=O)O)NC(=O)CNC(=O)C(CCCNC(=N)N)NC1=O)NC(=O)CCCC1C(=O)C=CC1=O)C(=O)N[C@@H](C)C(=O)Nc1cc(C[C@H](N)CCC(=O)O)ccc1O]

To a stirred solution of 302 (71 mg, 0.047 mmol) in DCM (3 mL) at room temperature was added TFA (3 mL). The mixture was stirred for 2 h at room temperature, concentrated to afford 303 (60 mg, 100% yield) as a light-yellow liquid.

### Example 212. Synthesis of benzyl ((S)-17-(((benzyloxy) carbonyl)amino)-1-(4-((4-(tert-butoxy)-4-oxobutyl) carbamoyl)phenyl)-1, 11-dioxo-5, 8-dioxa-2, 12-diazaoctadecan-18-oyl)-L-valinate (307)

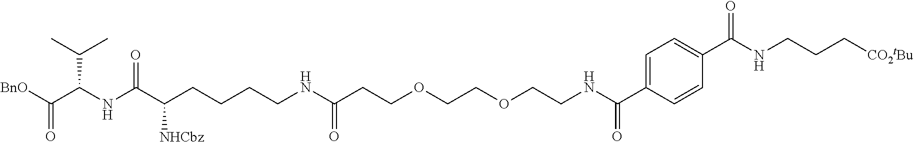 [SMILES: CC(C)[C@H](NC(=O)[C@H](CCCCNC(=O)CCOCCOCCNC(=O)c1ccc(C(=O)NCCCC(=O)OC(C)(C)C)cc1)NC(=O)OCc1ccccc1)C(=O)OCc1ccccc1]

To a stirred solution of 9 (4.5 g, 5.79 mmol) in DCM (20 mL) at room temperature were added 306 (1.10 g, 5.79 mmol), HATU (2.64 g, 6.95 mmol) and DIEA (1.50 g, 11.58 mmol). The mixture was stirred at room temperature for 2 h, diluted with DCM (50 mL), washed with water (50 mL), brine (50 mL), concentrated. The residue was purified by a silica gel column, eluted with DCM and methanol, to afford 307 (4.4 g, 83% yield) as a colorless oil.

### Example 213. Synthesis of 4-(4-(((4S, 7S)-7-(((benzyloxy) carbonyl)amino)-4-isopropyl-3, 6, 13-trioxo-1-phenyl-2, 16, 19-trioxa-5, 12-diazahenicosan-21-yl) carbamoyl)benzamido) butanoic acid (308)

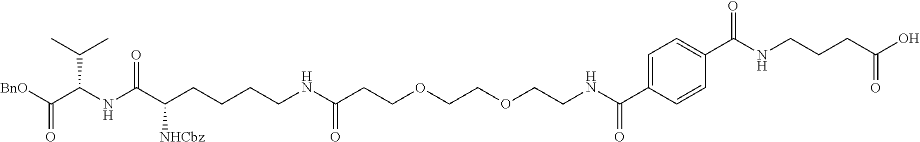 [SMILES: CC(C)[C@H](NC(=O)[C@H](CCCCNC(=O)CCOCCOCCNC(=O)c1ccc(C(=O)NCCCC(=O)O)cc1)NC(=O)OCc1ccccc1)C(=O)OCc1ccccc1]

To a stirred solution of 307 (2.3 g, 2.50 mmol) in DCM (10 mL) at room temperature was added TFA (15 mL). The reaction mixture was stirred at room temperature for 2 h, and concentrated. The residue was purified by a silica gel column, eluted with DCM and methanol, to afford 308 (1.3 g, 60% yield) as a white solid.

### Example 214. Synthesis of benzyl ((S)-1-(4-((4-((4-((2S, 5S, 115, 14R)-14-benzyl-11-(2-(tert-butoxy)-2-oxoethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(((benzyloxy) carbonyl)amino)-1, 11-dioxo-5, 8-dioxa-2, 12-diazaoctadecan-18-oyl)-L-valinate (309)

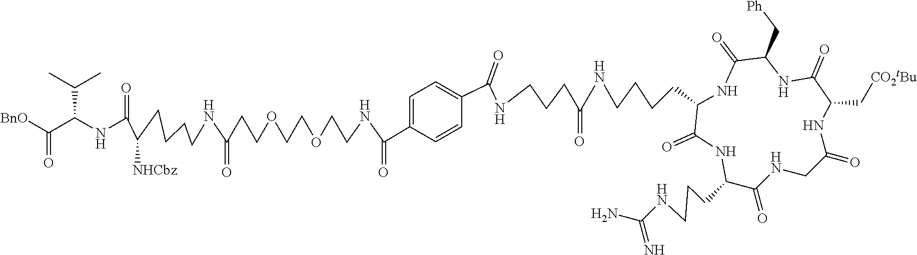 [SMILES: CC(C)[C@H](NC(=O)[C@H](CCCCNC(=O)CCOCCOCCNC(=O)c1ccc(C(=O)NCCCC(=O)NCCCC[C@@H]2NC(=O)[C@@H](Cc3ccccc3)NC(=O)[C@H](CC(=O)OC(C)(C)C)NC(=O)CNC(=O)[C@H](CCCNC(=N)N)NC2=O)cc1)NC(=O)OCc1ccccc1)C(=O)OCc1ccccc1]

To a stirred solution of 308 (870 mg, 1.00 mmol) in DMF (5 mL) at room temperature were added 291 (799 mg, 1.21 mmol), HATU (570 mg, 1.50 mmol) and DIEA (1030 mg, 8.00 mmol). The mixture was stirred at room temperature for 1 h and then purified by prep-HPLC, lyophilized to afford 309 (480 mg, 32% yield) as a white solid.

### Example 215. Synthesis of ((S)-17-amino-1-(4-((4-((4-((2S, 5S, 11S, 14R)-14-benzyl-11-(2-(tert-butoxy)-2-oxoethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-1, 11-dioxo-5, 8-dioxa-2, 12-diazaoctadecan-18-oyl)-L-valine (310)

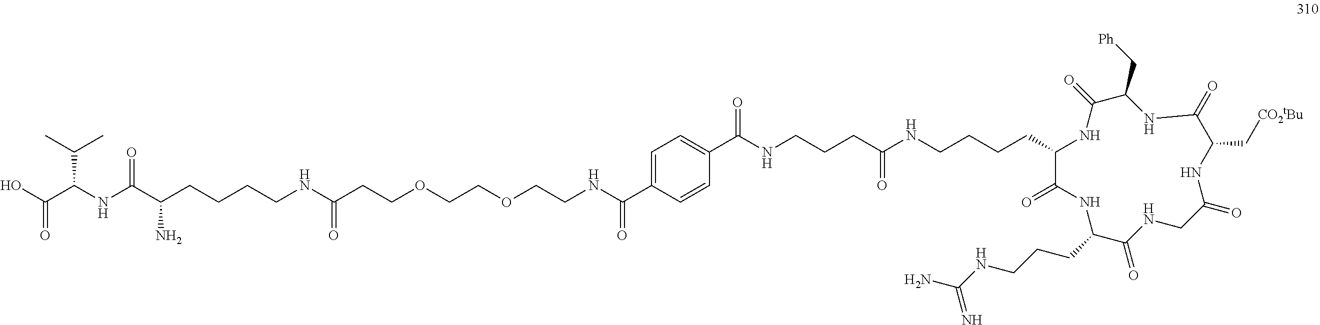 [SMILES: CC(C)[C@H](NC(=O)[C@@H](N)CCCCNC(=O)CCOCCOCCNC(=O)c1ccc(C(=O)NCCCC(=O)NCCCC[C@@H]2NC(=O)[C@@H](Cc3ccccc3)NC(=O)[C@H](CC(=O)OC(C)(C)C)NC(=O)CNC(=O)[C@H](CCCNC(=N)N)NC2=O)cc1)C(=O)O]

To a stirred solution of 309 (480 mg, 0.32 mmol) in isopropyl alcohol (100 mL) at room temperature was added Pd/C (200 mg, 10 wt %). The reaction flask was evacuated and back filled with hydrogen for three times, and the mixture reaction was stirred under a hydrogen balloon at 50° C. for 8 h, filtered, concentrated to afford 310 (289 mg, 100% yield) as a brown solid.

### Example 216. Synthesis of ((S)-1-(4-((4-((4-((2S, 5S, 11S, 14R)-14-benzyl-11-(2-(tert-butoxy)-2-oxoethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(4-(2, 5-dioxo-2, 5-dihydro-1H-pyrrol-1-yl) butanamido)-1, 11-dioxo-5, 8-dioxa-2, 12-diazaoctadecan-18-oyl)-L-valine (311)

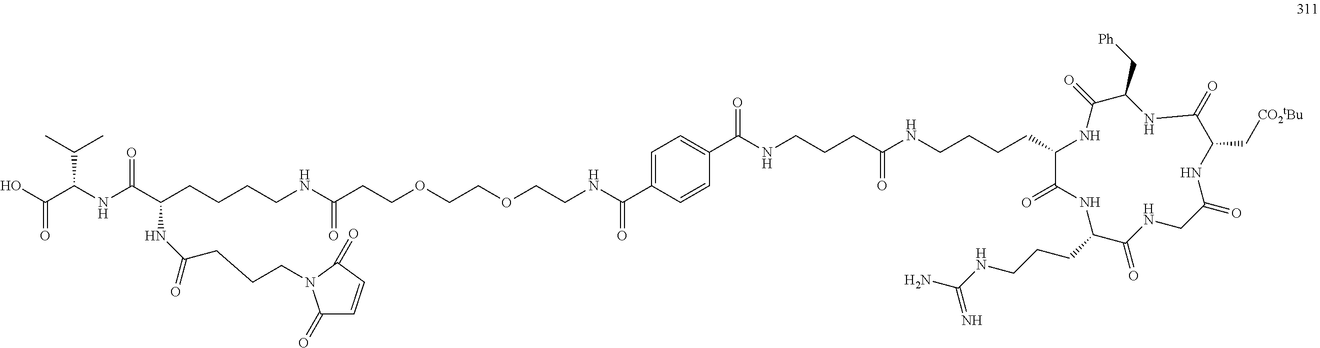 [SMILES: CC(C)[C@H](NC(=O)[C@H](CCCCNC(=O)CCOCCOCCNC(=O)c1ccc(C(=O)NCCCC(=O)NCCCC[C@@H]2NC(=O)[C@@H](Cc3ccccc3)NC(=O)[C@H](CC(=O)OC(C)(C)C)NC(=O)CNC(=O)[C@H](CCCNC(=N)N)NC2=O)cc1)NC(=O)CCCN1C(=O)C=CC1=O)C(=O)O]

To a solution of compound 310 (289 mg, 0.200 mmol) in DMF (5 mL) at room temperature were added 19 (84.0 mg, 0.300 mmol) and DIEA (51.0 mg, 0.400 mmol). The reaction mixture was stirred for 1 h, purified by prep-HPLC, lyophilized to afford 311 (110 mg, 38% yield) as a white solid.

### Example 217. Synthesis of tert-butyl (R)-5-(3-((17S, 20S, 23S)-1-(4-((4-((4-((2S, 5S, 11S, 14R)-14-benzyl-11-(2-(tert-butoxy)-2-oxoethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)-4-hydroxyphenyl)-4-((tert-butoxycarbonyl)amino) pentanoate (312)

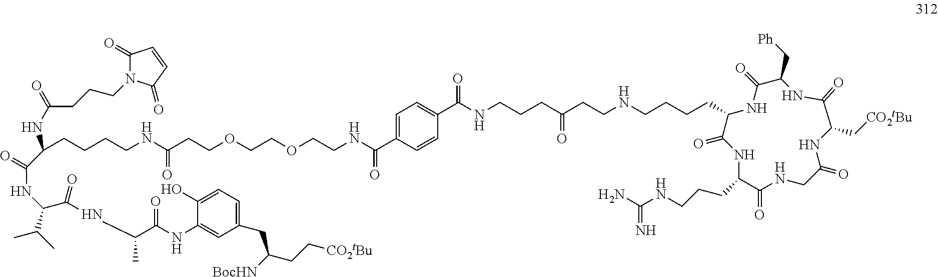 [SMILES: CC(C)[C@H](NC(=O)[C@H](CCCCNC(=O)CCOCCOCCNC(=O)c1ccc(C(=O)NCCCC(=O)CCNCCCC[C@@H]2NC(=O)[C@@H](Cc3ccccc3)NC(=O)[C@H](CC(=O)OC(C)(C)C)NC(=O)CNC(=O)[C@H](CCCNC(=N)N)NC2=O)cc1)NC(=O)CCCN1C(=O)C=CC1=O)C(=O)N[C@@H](C)C(=O)Nc1cc(C[C@@H](CCC(=O)OC(C)(C)C)NC(=O)OC(C)(C)C)ccc1O]

To a solution of compound 311 (82 mg, 0.056 mmol) in DMF (5 mL) at room temperature were added 21 (31.0 mg, 0.067 mmol), HATU (39.0 mg, 0.100 mmol) and DIEA (11 mg, 0.084 mmol). The mixture was stirred at room temperature for 1 h, concentrated, purified by prep-HPLC, lyophilized to afford 312 (40 mg, 38% yield) as a white solid.

### Example 218. Synthesis of (R)-4-amino-5-(3-((17S, 20S, 23S)-1-(4-((4-((4-((2S, 5S, 11S, 14R)-14-benzyl-1 1-(carboxymethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)-4-hydroxyphenyl) pentanoic acid (313)

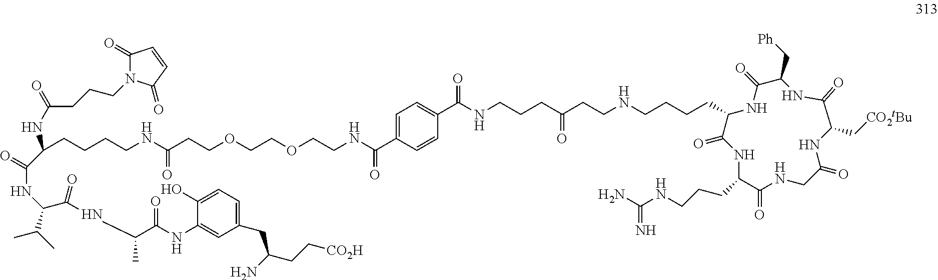 [SMILES: CC(C)[C@H](NC(=O)[C@H](CCCCNC(=O)CCOCCOCCNC(=O)c1ccc(C(=O)NCCCC(=O)CCNCCCC[C@@H]2NC(=O)[C@@H](Cc3ccccc3)NC(=O)[C@H](CC(=O)OC(C)(C)C)NC(=O)CNC(=O)[C@H](CCCNC(=N)N)NC2=O)cc1)NC(=O)CCCN1C(=O)C=CC1=O)C(=O)N[C@@H](C)C(=O)Nc1cc(C[C@H](N)CCC(=O)O)ccc1O]

To a solution of compound 312 (40 mg, 0.27 mmol) in DCM (3 mL) at room temperature was added TFA (4 mL). The mixture was stirred at room temperature for 2 hours and then concentrated to afford 313 (36 mg, 100% yield) as a light liquid.

### Example 219. Synthesis of (R)-5-(3-((17S, 20S, 23S)-1-(4-((4-((4-((2S, 5S, 11S, 14R)-14-benzyl-11-(carboxymethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)-4-hydroxyphenyl)-4-(2-((6S, 9R, 11R)-6-((S)-sec-butyl)-9-isopropyl-2, 3, 3, 8-tetramethyl-4, 7, 13-trioxo-12-oxa-2, 5, 8-triazatetradecan-11-yl) thiazole-4-carboxamido) pentanoic acid (314)

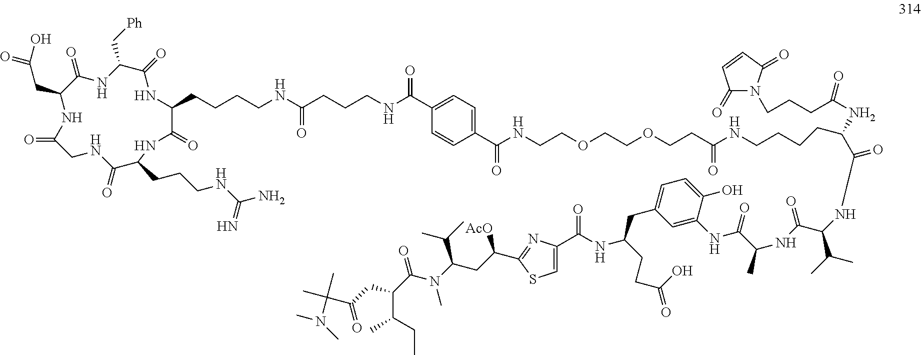

To a stirred solution of 313 (36 mg, 0.022 mmol) in DMF (5 mL) at 0° C. were added 24 (15.0 mg, 0.022 mmol) and DIEA (30 mg, 0.23 mmol). The reaction mixture was stirred at 0° C. for 0.5 h and at room temperature for 1 h, concentrated, purified by prep-HPLC, lyophilized to afford 314 (14 mg, 30% yield) as a white solid.

### Example 220. Synthesis of (S)-17-(((benzyloxy) carbonyl)amino)-2, 2-dimethyl-4, 11-dioxo-3, 8-dioxa-5, 12-diazaoctadecan-18-oic acid (317)

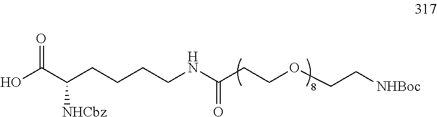

To a stirred solution of 316 (300 mg, 0.554 mmol) in DCM (10 mL) at room temperature were added NHS (96 mg, 0.831 mmol) and EDCI (212 mg, 1.108 mmol). The reaction mixture was stirred for 3 h at room temperature, and extracted with DCM (30 mL×2) and water (15 mL). The organic layers were combined and washed with brine (15 mL×2), dried over anhydrous sodium sulfate and concentrated in vacuo to afford a crude product. To the crude in DCM (10 mL) were added ((benzyloxy) carbonyl)-L-lysine (155 mg, 0.554 mmol) and DIEA (0.193 mL, 1.108 mmol). The mixture was stirred at room temperature overnight and concentrated in vacuo. The residue was purified by prep-HPLC to afford 317 (331 mg, 74% yield).

### Example 221. Synthesis of (S)-17-amino-2, 2-dimethyl-4, 11-dioxo-3, 8-dioxa-5, 12-diazaoctadecan-18-oic acid (318)

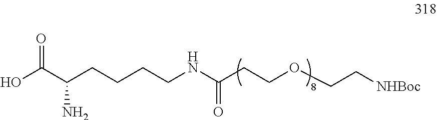

To a stirred solution of 317 (331 mg, 0.412 mmol) in THF (20 mL) at room temperature was added Pd/C (33 mg, 10 wt %). The reaction mixture was filtered over celite. The filtrate was concentrated in vacuo to afford 318 (262 mg, crude) as a colorless oil.

### Example 222. Synthesis of (S)-17-(4-(2, 5-dioxo-2, 5-dihydro-1H-pyrrol-1-yl) butanamido)-2, 2-dimethyl-4, 11-dioxo-3, 8-dioxa-5, 12-diazaoctadecan-18-oic acid (319)

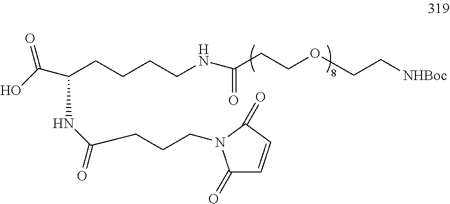

To a solution of compound 318 (262 mg, 0.391 mmol) in DCM (10 mL) were added 19 (155 mg, 0.587 mmol) and DIEA (101 mg, 0.782 mmol). The reaction mixture was stirred at room temperature for 2 h and concentrated in vacuo. The residue was purified by prep-HPLC to afford 319 (171 mg, 52% yield over two steps).

### Example 223. Synthesis of tert-butyl ((5S, 8S, 11S)-1-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)amino)-11-(4-(2, 5-dioxo-2, 5-dihydro-1H-pyrrol-1-yl) butanamido)-8-isopropyl-5-methyl-1, 4, 7, 10, 17-pentaoxo-20-oxa-3, 6, 9, 16-tetraazad° Cosan-22-yl) carbamate (320)

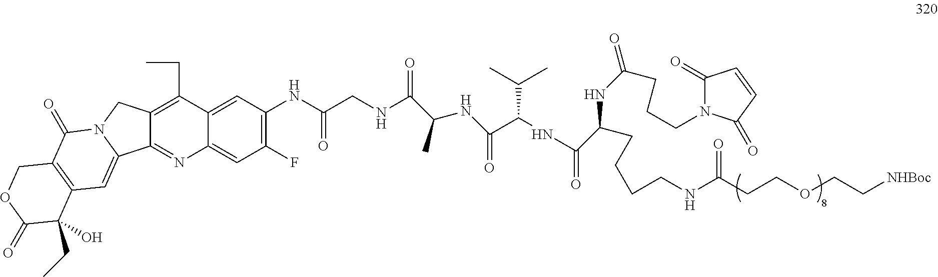

To a solution of compound 173 (120 mg, 0.188 mmol) in DCM (5 mL) were added 319 (157 mg, 0.188 mmol), HATU (86 mg, 0.226 mmol) and DIEA (97 mg, 0.754 mmol). The mixture was stirred at room temperature for 1 h and concentrated in vacuo. The residue was purified by column chromatography on silica gel (eluted with DCM: MeOH=10:1) to afford 320 (142 mg, 52% yield) as a yellow solid.

### Example 224. Synthesis of (S)-6-(3-(2-aminoethoxy) propanamido)-N—((S)-1-(((S)-1-((2-(((S)-4, 11-diethyl-8-fluoro-4-hydroxy-3, 14-dioxo-3, 4, 12, 14-tetrahydro-1H-pyrano [3′, 4′:6, 7]indolizino [1, 2-b]quinolin-9-yl)amino)-2-oxoethyl)amino)-1-oxopropan-2-yl)amino)-3-methyl-1-oxobutan-2-yl)-2-(4-(2, 5-dioxo-2, 5-dihydro-1H-pyrrol-1-yl) butanamido) hexanamide (321)

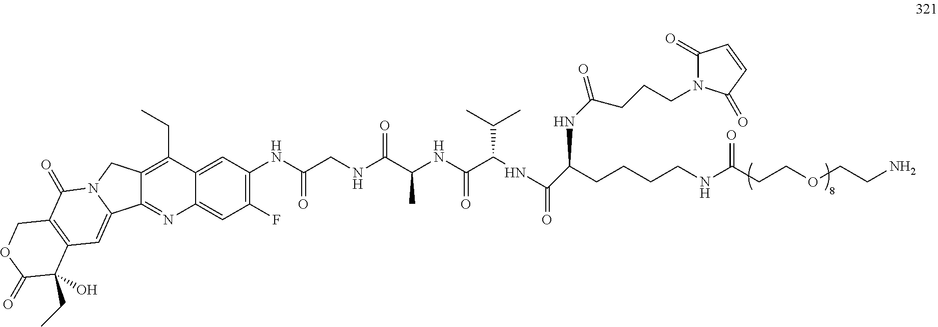

To a solution of compound 321 (142 mg, 0.098 mmol, 1.0 eq) in DCM (5 mL) was added TFA (2 mL). The mixture was stirred at room temperature for 1 h and concentrated in vacuo to afford 321 (132.22 mg, crude) as a yellow solid.

### Example 225. Synthesis of (18S, 21S, 24S)-28-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)amino)-18-(4-(2, 5-dioxo-2, 5-dihydro-1H-pyrrol-1-yl) butanamido)-21-isopropyl-24-methyl-5, 12, 19, 22, 25, 28-hexaoxo-3, 9-dioxa-6, 13, 20, 23, 26-pentaazaoctacosanoic acid (322)

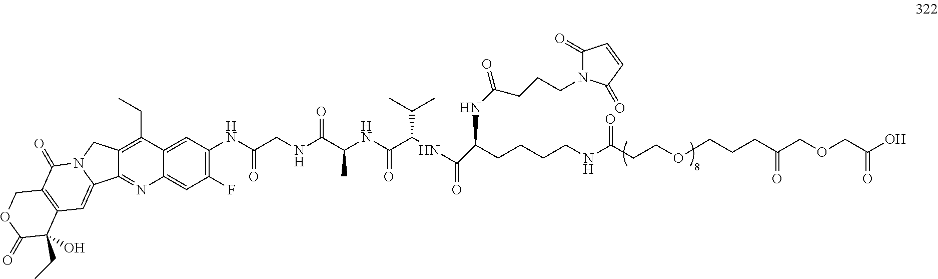

To a solution of compound 321 (132 mg, 0.098 mmol) in DCM (5 mL) were added 1, 4-dioxane-2, 6-dione (11 mg, 0.098 mmol) and DIEA (38 mg, 0.293 mmol, 3.0 eq). The mixture was stirred at room temperature for 0.5 h, and then concentrated in vacuo. The residue was purified by column chromatography on a silica gel column (eluted with DCM: MeOH=1:1) to afford 322 (76 mg, 53% yield over two steps) as a yellow solid.

### Example 226. Synthesis of tert-butyl 2-((2S, 5R, 8S, 11S)-5-benzyl-8-((5S, 8S, 11S)-1-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)amino)-11-(4-(2, 5-dioxo-2, 5-dihydro-1H-pyrrol-1-yl) butanamido)-8-isopropyl-5-methyl-1, 4, 7, 10, 17, 24, 28-heptaoxo-20, 26-dioxa-3, 6, 9, 16, 23, 29-hexaazatritriacontan-33-yl)-1 1-(3-guanidinopropyl)-3, 6, 9, 12, 15-pentaoxo-1, 4, 7, 10, 13-pentaazacyclopentadecan-2-yl)acetate (323)

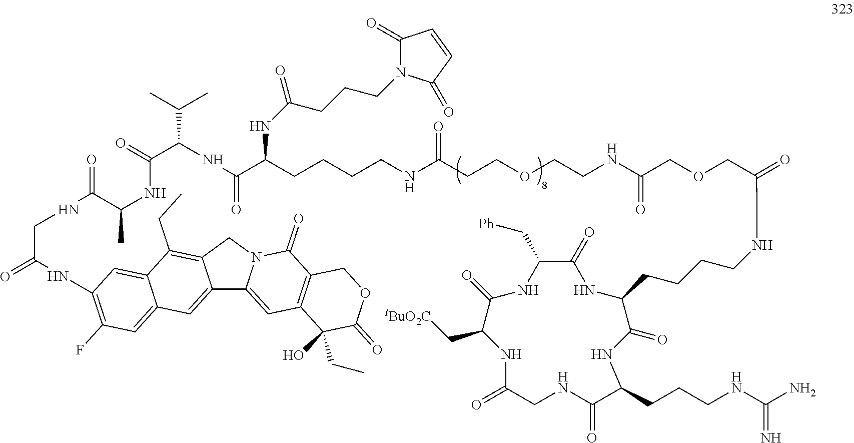

To a solution of compound 322 (60 mg, 0.041 mmol) in DMF (5 mL) were added 291 (40 mg, 0.061 mmol), (23 mg, 0.062 mmol) and DIEA (11 mg, 0.082 mmol). The mixture was stirred at room temperature for 0.5 h and concentrated in vacuo to afford 323 (86 mg, crude).

### Example 227. Synthesis of 2-((2S, 5R, 8S, 11S)-5-benzyl-8-((5S, 8S, 11S)-1-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)amino)-11-(4-(2, 5-dioxo-2, 5-dihydro-1H-pyrrol-1-yl) butanamido)-8-isopropyl-5-methyl-1, 4, 7, 10, 17, 24, 28-heptaoxo-20, 26-dioxa-3, 6, 9, 16, 23, 29-hexaazatritriacontan-33-yl)-11-(3- guanidinopropyl)-3, 6, 9, 12, 15-pentaoxo-1, 4, 7, 10, 13-pentaazacyclopentadecan-2-yl) acetic acid (324)

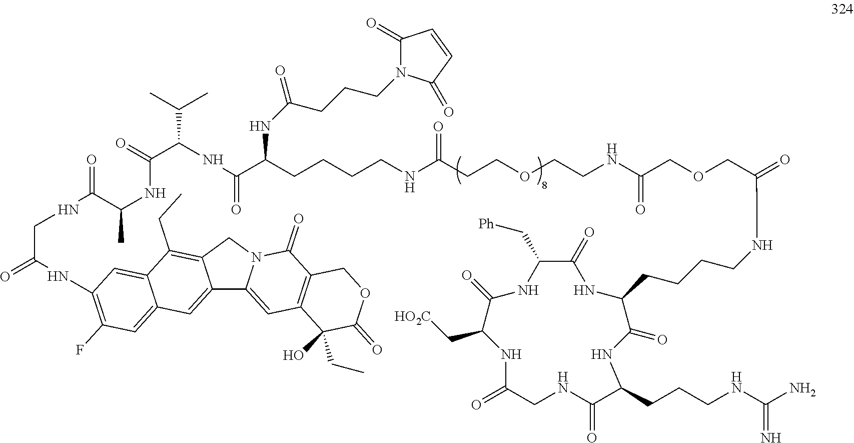

To a solution of compound 323 (86 mg, 0.041 mmol) in DCM (3 mL) was added TFA (2 mL). The mixture was stirred at room temperature for 1 h and concentrated in vacuo. The residue was purified by prep-HPLC to afford 324 (30 mg, 36% yield over two steps) as a white solid.

### Example 228. Synthesis of tert-butyl (R)-5-(((S)-1-(benzyloxy)-3-methyl-1-oxobutan-2-yl)amino)-4-(((benzyloxy) carbonyl)amino)-5-oxopentanoate (327)

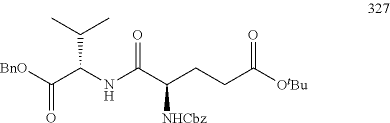 [SMILES: CC(C)[C@H](NC(=O)[C@@H](CCC(=O)OC(C)(C)C)NC(=O)OCc1ccccc1)C(=O)OCc1ccccc1]

To a solution of compound 326 (7.0 g, 33.77 mmol) in DCM (200 mL) at room temperature were added H-Val-OBn (11.3 g, 33.49 mmol), HATU (15.4 g, 40.51 mmol) and DIEA (8.7 g, 67.54 mmol).

The reaction mixture was stirred for 1 h, diluted with DCM (400 mL), washed with water (300 mL), 5% Na₂CO₃ (300 mL), 1 M HCl (300 mL), and brine (300 mL), concentrated to give a crude product, which was purified by a silica gel column, eluted with petroleum ether and ethyl acetate, to afford 327 (16.7 g, 94% yield) as a white solid.

### Example 229. Synthesis of (R)-5-(((S)-1-(benzyloxy)-3-methyl-1-oxobutan-2-yl)amino)-4-(((benzyloxy) carbonyl)amino)-5-oxopentanoic acid (328)

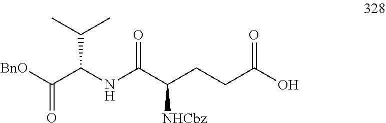 [SMILES: CC(C)[C@H](NC(=O)[C@@H](CCC(=O)O)NC(=O)OCc1ccccc1)C(=O)OCc1ccccc1]

To a solution of compound 327 (9.3 g, 17.75 mmol) in DCM (40 mL) at room temperature was added TFA (40 mL). The mixture was stirred at room temperature for 2 h, and concentrated. The residue was triturated with methyl tert-butyl ether, filtered and the solid was dried under vacuum to afford 328 (8.3 g, 100% yield) as a white solid.

### Example 230. Synthesis of tert-butyl (R)-5-(((S)-1-(benzyloxy)-3-methyl-1-oxobutan-2-yl) carbamoyl)-3, 8-dioxo-1-phenyl-2, 12-dioxa-4, 9-diazapentadecan-15-oate (330)

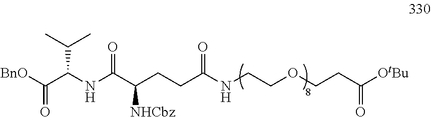

To a solution of compound 328 (2.65 g, 5.62 mmol) in DCM (100 mL) at room temperature were added 329 (2.80 g, 5.62 mmol), HATU (2.60 g, 6.75 mmol) and DIEA (1.0 g, 8.44 mmol). The mixture was stirred at room temperature for 1 h, diluted with DCM (100 mL), washed with water (100 mL), 5% Na₂CO₃ (100 mL), 1 M HCl (100 mL) and brine (100 mL), then concentrated. The residue was purified by a silica gel column, eluted with DCM and methanol, to afford 330 (5.4 g, 100% yield) as a gray solid.

### Example 231. Synthesis of N5-(2-(3-(tert-butoxy)-3-oxopropoxy)ethyl)-D- glutaminyl-L-valine (331)

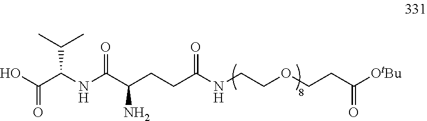

To a solution of compound 330 (5.4 g, 5.68 mmol) in isopropyl alcohol (100 mL) at room temperature was added Pd/C (1.2 g, 10 wt %). the reaction flask was evacuated and back filled with hydrogen for three times, the mixture reaction was stirred overnight under hydrogen balloon at room temperature, filtered, concentrated to afford 331 (4.2 g, 100% yield) as a colorless oil.

### Example 232. Synthesis of N5-(2-(3-(tert-butoxy)-3-oxopropoxy)ethyl)-N2-(4-(2, 5-dioxo-2, 5-dihydro-1H-pyrrol-1-yl) butanoyl)-D- glutaminyl-L-valine (332)

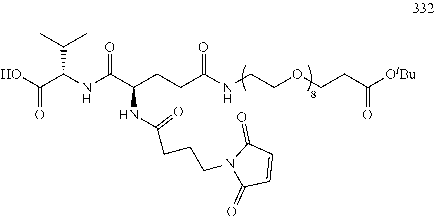

To a solution of compound 331 (4.1 g, 5.64 mmol) in DMF (15 mL) at room temperature were added 19 (2.00 g, 7.34 mmol) and DIEA (1.50 g, 11.30 mmol), the reaction mixture was stirred for 1 h at room temperature, purified by prep-HPLC, lyophilized to afford 332 (2.60 g, 51% yield) as a white solid.

### Example 233. Synthesis of tert-butyl ((S)-1-((4-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy) ethoxy)methyl)phenyl)amino)-1-oxopropan-2-yl) carbamate (334)

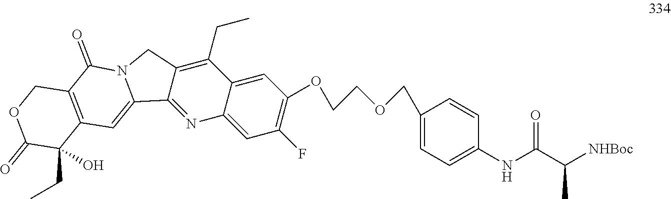 [SMILES: CCc1c2c(nc3cc(F)c(OCCOCc4ccc(NC(=O)[C@H](C)NC(=O)OC(C)(C)C)cc4)cc13)-c1cc3c(c(=O)n1C2)COC(=O)[C@]3(O)CC]

To a solution of compound 333 (454 mg, 1.00 mmol) and compound 178 (294 mg, 1.00 mmol) in THF (20 mL) were added PPh₃ (525 mg, 2.00 mmol) and DEAD (350 mg, 0.182 mmol). The mixture was stirred at room temperature for 5 h. Diluted with DCM (100 mL), washed with water (100 mL) brine (100 mL), concentrated to give the crude, which was purified by a silica gel column, eluted with DCM and methanol, concentrated to afford 334 (423 mg, 58% yield) as a gray solid.

### Example 234. Synthesis of (S)-2-amino-N-(4-((2-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)oxy) ethoxy)methyl)phenyl) propanamide (335)

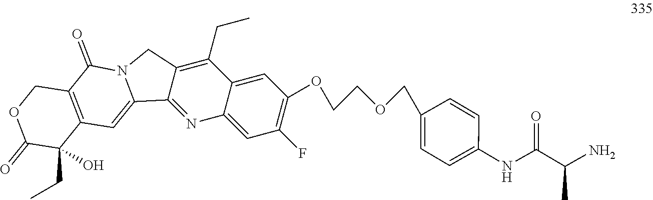 [SMILES: CCc1c2c(nc3cc(F)c(OCCOCc4ccc(NC(=O)[C@H](C)N)cc4)cc13)-c1cc3c(c(=O)n1C2)COC(=O)[C@]3(O)CC]

To a solution of compound 334 (420 mg, 0.57 mmol) in methanol (10 mL) at room temperature was added HCl (4 M). The reaction mixture was stirred for 1 h, concentrated to afford 335 (360 mg, 100% yield) as a white solid.

### Example 235. Synthesis of tert-butyl (2S, 5S, 8R)-1-((4-((2-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)oxy) ethoxy)methyl)phenyl)amino)-8-(4-(2, 5-dioxo-2, 5-dihydro-1H-pyrrol-1-yl) butanamido)-5-isopropyl-2-methyl-1, 4, 7, 11-tetraoxo-15-oxa-3, 6, 12-triazaoctadecan-18-oate (336)

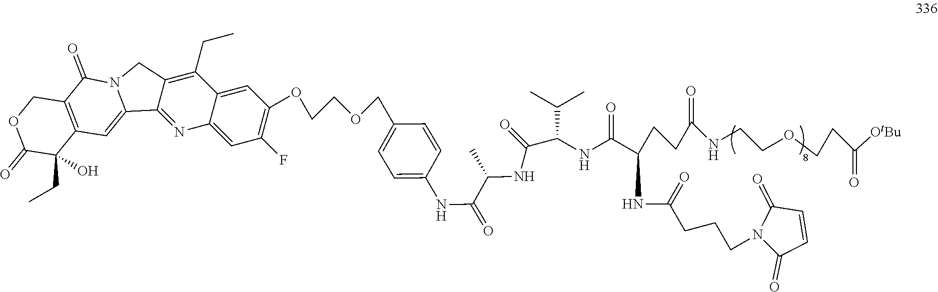

To a solution of compound 332 (650 mg, 0.70 mmol) in DMF (10 mL) at room temperature were added 335 (460 mg, 0.72 mmol), HATU (411 mg, 1.08 mmol), and DIEA (1.50 g, 11.30 mmol). The mixture was stirred at room temperature for 1 h, purified by prep-HPLC, lyophilized to afford 336 (2.60 g, 510% yield) as a yellow solid.

### Example 236. Synthesis of (2S, 5S, 8R)-1-((4-((2-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)oxy) ethoxy)methyl)phenyl)amino)-8-(4-(2, 5-dioxo-2, 5-dihydro-1H-pyrrol-1-yl) butanamido)-5-isopropyl-2-methyl-1, 4, 7, 11-tetraoxo-15-oxa-3, 6, 12-triazaoctadecan-18-oic acid (337)

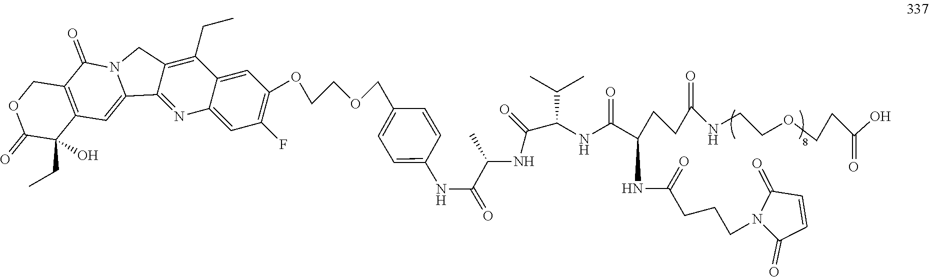

To a solution of compound 336 (312 mg, 0.20 mmol) in DCM (5 mL) at room temperature was added TFA (5 mL). The mixture was stirred at room temperature for 1 h and concentrated. The residue was triturated with methyl tert-butyl ether, filtered and the solid was dried under vacuum to afford 337 (360 mg, 83% yield) as a yellow solid.

### Example 237. Synthesis of tert-butyl 2-((2S, 5R, 8S, 1 1S)-5-benzyl-8-((2S, 5S, 8R)-1-((4-(2-(((S)-4, 11-diethyl-8-fluoro-4-hydroxy-3, 14-dioxo-3, 4, 12, 14-tetrahydro-1H-pyrano [3′, 4′:6, 7]indolizino[l, 2-b]quinolin-9-yl)oxy) ethoxy)phenyl)amino)-8-(4-(2, 5-dioxo-2, 5-dihydro-1H-pyrrol-1-yl) butanamido)-5-isopropyl-2-methyl-1, 4, 7, 11, 18-pentaoxo-15-oxa-3, 6, 12, 19-tetraazatricosan-23-yl)-11-(3- guanidinopropyl)-3, 6, 9, 12, 15-pentaoxo-1, 4, 7, 10, 13-pentaazacyclopentadecan-2-yl)acetate (338)

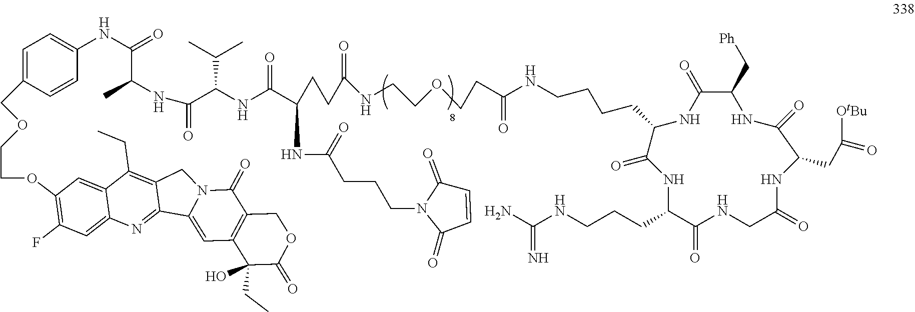

To a solution of compound 337 (59 mg, 0.089 mmol) in DMF (5 mL) at room temperature were added 291 (129 mg, 0.089 mmol), HATU (51 mg, 0.13 mmol), and DIEA (23 mg, 0.17 mmol). The mixture was stirred at room temperature for 1 h, purified by prep-HPLC, lyophilized to afford 338 (118 mg, 63% yield) as a yellow solid.

### Example 238. Synthesis of 2-((2S, 5R, 8S, 11S)-5-benzyl-8-((2S, 5S, 8R)-1-((4-(2-(((S)-4, 11-diethyl-8-fluoro-4-hydroxy-3, 14-dioxo-3, 4, 12, 14-tetrahydro-1H-pyrano [3′, 4′:6, 7]indolizino [1,2-b]quinolin-9-yl)oxy) ethoxy)phenyl)amino)-8-(4-(2, 5-dioxo-2, 5-dihydro-1H-pyrrol-1-yl) butanamido)-5-isopropyl-2-methyl-1, 4, 7, 11, 18-pentaoxo-15-oxa-3, 6, 12, 19-tetraazatricosan-23-yl)-11-(3-guanidinopropyl)-3, 6, 9, 12, 15-pentaoxo-1, 4, 7, 10, 13-pentaazacyclopentadecan-2-yl) acetic acid (339)

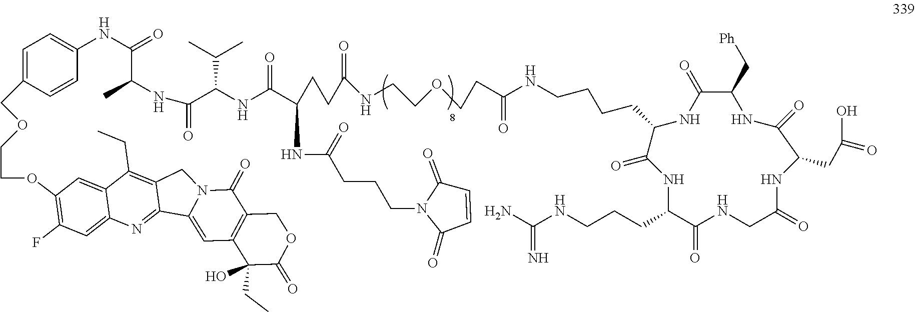

To a solution of compound 338 (118 mg, 0.056 mmol) in DCM (3 mL) at room temperature was added TFA (3 mL). The mixture was stirred at room temperature for 2 h, purified by prep-HPLC, lyophilized to afford 339 (17 mg, 15% yield) as a yellow solid.

### Example 239. Synthesis of 1-(4-((17S, 20S, 23S)-1-(4-((4-((4-((2S, 5S, 11S, 14R)-14-benzyl-11-(2-(tert-butoxy)-2-oxoethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (341)

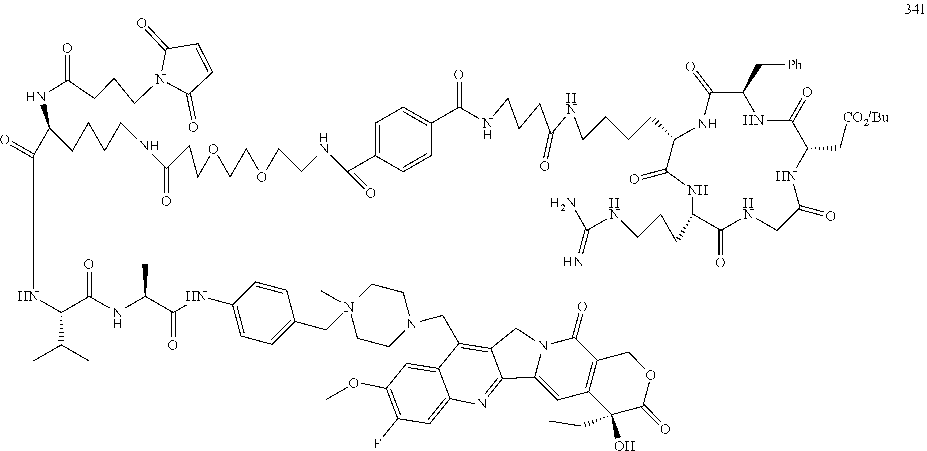 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)[C@@H](NC(=O)[C@H](CCCCNC(=O)CCOCCOCCNC(=O)c3ccc(C(=O)NCCCC(=O)NCCCC[C@@H]4NC(=O)[C@@H](Cc5ccccc5)NC(=O)[C@H](CC(=O)OC(C)(C)C)NC(=O)CNC(=O)[C@H](CCCNC(=N)N)NC4=O)cc3)NC(=O)CCCN3C(=O)C=CC3=O)C(C)C)cc2)CC1]

To a solution of compound 311 (79 mg, 0.055 mmol) in DMF (3 mL) at 0° C. were added 185 (45 mg, 0.066 mmol), HATU (32 mg, 0.08 mmol) and DIEA (30 mg, 0.23 mmol). The mixture was stirred at 0° C. for 1 h. purified by prep-HPLC, lyophilized to afford 341 (29 mg, 25% yield) as a white solid.

### Example 240. Synthesis of 1-(4-((17S, 20S, 23S)-1-(4-((4-((4-((2S, 5S, 11S, 14R)-14-benzyl-11-(carboxymethyl)-5-(3- guanidinopropyl)-3, 6, 9, 12, 15-pentaoxo-1, 4, 7, 10, 13-pentaazacyclopentadecan-2-yl)butyl)amino)-4-oxobutyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)benzyl)-4-(((S)-4-ethyl-8-fluoro-4-hydroxy-9-methoxy-3, 14-dioxo-3, 4, 12, 14-tetrahydro-IH-pyrano[3′, 4′:6, 7]indolizino[1, 2-b]quinolin-11-yl)methyl)-1-methylpiperazin-1-ium (342)

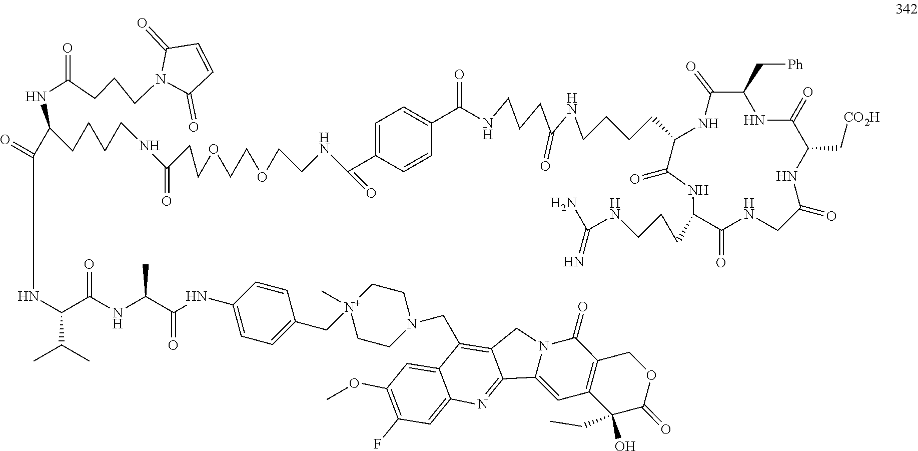 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)[C@@H](NC(=O)[C@H](CCCCNC(=O)CCOCCOCCNC(=O)c3ccc(C(=O)NCCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)cc3)NC(=O)CCCN3C(=O)C=CC3=O)C(C)C)cc2)CC1]

To a solution of compound 341 (39 mg, 0.018 mmol) in DCM (2 mL) at room temperature was added TFA (2 mL). The mixture was stirred at room temperature for 2 h and concentrated. The residue was purified by prep-HPLC, lyophilized to afford 342 (17 mg, 45% yield) as a white solid.

### Example 241. Synthesis of N2-((benzyloxy) carbonyl)-N6-(4-(tert-butoxycarbonyl)benzoyl)-L-lysine (344)

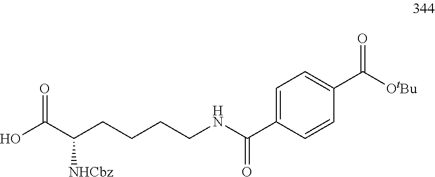 [SMILES: CC(C)(C)OC(=O)c1ccc(C(=O)NCCCC[C@H](NC(=O)OCc2ccccc2)C(=O)O)cc1]

To a stirred solution of ((benzyloxy) carbonyl)-L-lysine (30.2 g, 108.02 mmol) in water (200 mL) at 0° C. were added sodium hydroxide (4.3 g, 108.028 mmol), sodium bicarbonate (11.4 g, 108.028 mmol). Compound 5 (13.0 g, 54.01 mmol) in THF (100 mL) was added by dropwise at 0° C., and the resulting mixture was stirred at 0° C. for 3 h, diluted with ethyl acetate (100 mL) and water (100 mL), the aqueous layer was separated and washed with ethyl acetate (100 mL), acidified using 1M HCl (100 mL), extracted with DCM (400 mL×2). The organic layers were combined and washed with brine (200 mL), dried over anhydrous sodium sulfate, filtered, and concentrated to afford 344 (17.8 g, 68% yield) as a brown liquid.

### Example 242. Synthesis of benzyl (5S, 8S, 11S, 14S)-5-(4-(4-(tert-butoxycarbonyl)benzamido)butyl)-8, 11, 14-trimethyl-3, 6, 9, 12-tetraoxo-1-phenyl-2-oxa-4, 7, 10, 13-tetraazapentadecan-15-oate (346)

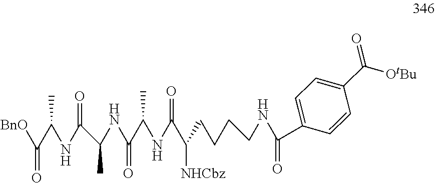 [SMILES: C[C@H](NC(=O)[C@H](C)NC(=O)[C@H](CCCCNC(=O)c1ccc(C(=O)OC(C)(C)C)cc1)NC(=O)OCc1ccccc1)C(=O)N[C@@H](C)C(=O)OCc1ccccc1]

To a stirred solution of 345 (2.1 g, 6.81 mmol) in DMF (80 mL) at room temperature were added 344 (3.3 g, 6.81 mmol), HATU (3.8 g, 10.21 mmol) and DIEA (5.2 g, 40.23 mmol), and the resulting mixture was stirred at room temperature for 1.5 h. Water (100 mL) was added and a solid crushed out, which was collected by filtration and dried under vacuum to afford 346 (5.6 g, 100% yield) as a light yellow solid.

### Example 243. Synthesis of 4-(((4S, 7S, 10S, 13S)-13-(((benzyloxy) carbonyl)amino)-4, 7, 10-trimethyl-3, 6, 9, 12-tetraoxo-1-phenyl-2-oxa-5, 8, 11-triazaheptadecan-17-yl) carbamoyl)benzoic acid (347)

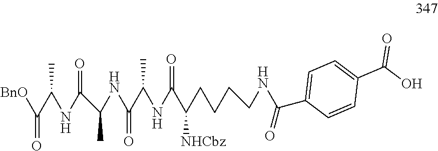 [SMILES: C[C@H](NC(=O)[C@H](C)NC(=O)[C@H](CCCCNC(=O)c1ccc(C(=O)O)cc1)NC(=O)OCc1ccccc1)C(=O)N[C@@H](C)C(=O)OCc1ccccc1]

To a stirred solution of 346 (5.3 g, 7.0 mmol) in DCM (50 mL) at room temperature was added TFA (45 mL), and the resulting mixture was heated to 40° C., then stirred overnight, concentrated. The residue was dissolved in DCM (20 mL) and methanol (2 mL), triturated with petroleum ether (60 mL), filtered, and dried under vacuum to afford 347 (4.7 g, 95% yield) as a light yellow solid.

### Example 244. Synthesis of tert-butyl (3, 14-dioxo-1-phenyl-2, 7, 10-trioxa-4, 13-diazahenicosan-21-yl) carbamate (350)

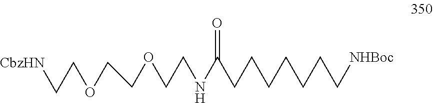 [SMILES: CC(C)(C)OC(=O)NCCCCCCCC(=O)NCCOCCOCCNC(=O)OCc1ccccc1]

To a stirred solution of 349 (8.3 g, 29.39 mmol) in THF (60 mL) at room temperature were added 348 (8.3 g, 32.33 mmol), HATU (18.8 g, 49.44 mmol) and DIEA (7.6 g, 58.79 mmol). The resulting mixture was stirred at room temperature for 1 h, diluted with DCM (200 mL), washed with water (200 mL), 5% sodium carbonate (200 mL), 1 M HCl (150 mL), brine (200 mL), dried over anhydrous sodium sulfate, filtered and concentrated. The residue was purified by a silica gel column, eluted with DCM and methanol, to give 350 (11.3 g, 73% yield) as a colorless oil.

### Example 245. Synthesis of benzyl (2-(2-(2-(8-aminooctanamido) ethoxy) ethoxy)ethyl) carbamate (351)

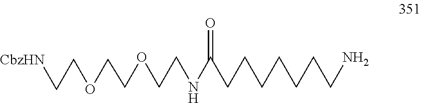 [SMILES: NCCCCCCCC(=O)NCCOCCOCCNC(=O)OCc1ccccc1]

To a stirred solution of 350 (10.0 g, 19.00 mmol) in DCM (50 mL) at room temperature was added TFA (25 mL), and the resulting mixture was stirred at room temperature for 1 h, concentrated to afford 351 (8.1 g, 100% yield) as a colorless oil.

### Example 246. Synthesis of tri-tert-butyl (26S, 30S)-3, 14, 23, 28-tetraoxo-1-phenyl-2, 7, 10-trioxa-4, 13, 22, 27, 29-pentaazadotriacontane-26, 30, 32-tricarboxylate (352)

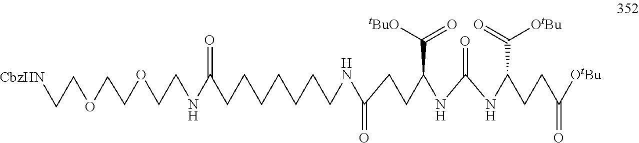 [SMILES: CC(C)(C)OC(=O)CC[C@H](NC(=O)N[C@@H](CCC(=O)NCCCCCCCC(=O)NCCOCCOCCNC(=O)OCc1ccccc1)C(=O)OC(C)(C)C)C(=O)OC(C)(C)C]

To a stirred solution of 351 (2.77 g, 6.55 mmol) in DCM (50 mL) at room temperature were added 13 (3.2 g, 6.55 mmol), HATU (2.74 g, 7.20 mmol) and DIEA (1.68 g, 13 mmol). The resulting mixture was stirred at room temperature for 1 h, diluted with DCM (200 mL), washed with water (200 mL), 5% sodium carbonate (200 mL), 1 M HCl (150 mL), brine (200 mL), dried over anhydrous sodium sulfate, filtered and concentrated. The residue was purified by prep-HPLC and lyophilized to give 352 (3.50 g, 63% yield) as a colorless oil.

### Example 247. Synthesis of tri-tert-butyl (22S, 26S)-1-amino-10, 19, 24-trioxo-3, 6-dioxa-9, 18, 23, 25-tetraazaoctacosane-22, 26, 28-tricarboxylate (353)

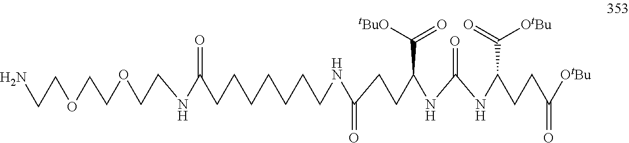 [SMILES: CC(C)(C)OC(=O)CC[C@H](NC(=O)N[C@@H](CCC(=O)NCCCCCCCC(=O)NCCOCCOCCN)C(=O)OC(C)(C)C)C(=O)OC(C)(C)C]

To a stirred solution of 352 (3.2 g, 3.57 mmol) in methanol (150 mL) at 40° C. was added Pd/C (1.5 g, 10 wt %), and the reaction flask was evacuated and back filled with hydrogen for three times, and then stirred overnight under a hydrogen balloon at 40° C., filtered and concentrated. The residue was purified by a silica gel column, eluted with DCM and methanol, to afford 353 (1.88 g, 69% yield) as a brown solid.

### Example 248. Synthesis of tri-tert-butyl (24S, 28S)-1-(4-(((4S, 7S, 10S, 13S)-13-(((benzyloxy) carbonyl)amino)-4, 7, 10-trimethyl-3, 6, 9, 12-tetraoxo-1-phenyl-2-oxa-5, 8, 11-triazaheptadecan-17-yl) carbamoyl)phenyl)-1, 12, 21, 26-tetraoxo-5, 8-dioxa-2, 11, 20, 25, 27-pentaazatriacontane-24, 28, 30-tricarboxylate (354)

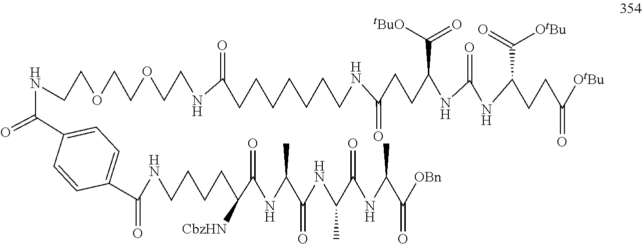 [SMILES: C[C@H](NC(=O)[C@H](C)NC(=O)[C@H](CCCCNC(=O)c1ccc(C(=O)NCCOCCOCCNC(=O)CCCCCCCNC(=O)CC[C@H](NC(=O)N[C@@H](CCC(=O)OC(C)(C)C)C(=O)OC(C)(C)C)C(=O)OC(C)(C)C)cc1)NC(=O)OCc1ccccc1)C(=O)N[C@@H](C)C(=O)OCc1ccccc1]

To a stirred solution of 353 (1.8 g, 2.36 mmol) in DMF (15 mL) at room temperature were added 347 (1.7 g, 2.36 mmol), PyBOP (1.85 g, 3.55 mmol) and DIEA (0.61 g, 4.74 mmol). The resulting mixture was stirred at room temperature for 1.5 h, purified by prep-HPLC, and lyophilized to give 354 (1.7 g, 49% yield) as a white solid.

### Example 249. Synthesis of N6-(4-(((22S, 26S)-22, 26-bis(tert-butoxycarbonyl)-31, 31-dimethyl-10, 19, 24, 29-tetraoxo-3, 6, 30-trioxa-9, 18, 23, 25-tetraazadotriacontyl) carbamoyl)benzoyl)-L-lysyl-L-alanyl-L-alanyl-L-alanine (355)

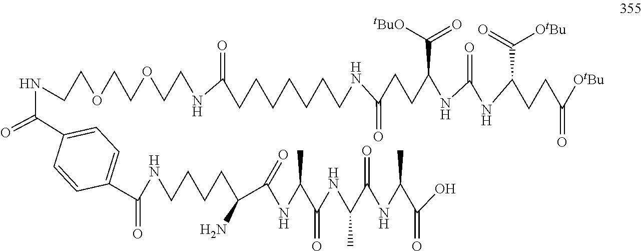 [SMILES: C[C@H](NC(=O)[C@H](C)NC(=O)[C@H](C)NC(=O)[C@@H](N)CCCCNC(=O)c1ccc(C(=O)NCCOCCOCCNC(=O)CCCCCCCNC(=O)CC[C@H](NC(=O)N[C@@H](CCC(=O)OC(C)(C)C)C(=O)OC(C)(C)C)C(=O)OC(C)(C)C)cc1)C(=O)O]

To a stirred solution of 354 (700 mg, 3.57 mmol) in methanol (50 mL) at room temperature was added Pd/C (500 mg, 10 wt %), the reaction flask was evacuated and back filled with hydrogen for three times, and then stirred under a hydrogen balloon at room temperature for 2 h, filtered and concentrated to afford 355 (560 mg, 94% yield) as a brown solid.

### Example 250. Synthesis of N6-(4-(((22S, 26S)-22, 26-bis(tert-butoxycarbonyl)-31, 31-dimethyl-10, 19, 24, 29-tetraoxo-3, 6, 30-trioxa-9, 18, 23, 25-tetraazadotriacontyl) carbamoyl)benzoyl)-N2-(4-(2, 5-dioxo-2, 5-dihydro-1H-pyrrol-1-yl) butanoyl)-L-lysyl-L-alanyl-L-alanyl-L-alanine (356)

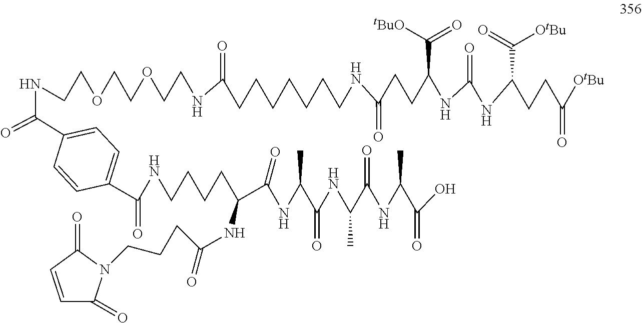 [SMILES: C[C@H](NC(=O)[C@H](C)NC(=O)[C@H](C)NC(=O)[C@H](CCCCNC(=O)c1ccc(C(=O)NCCOCCOCCNC(=O)CCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(=O)O]

To a stirred solution of 355 (560 mg, 0.45 mmol) in DMF (6 mL) at room temperature were added 19 (142 mg, 0.54 mmol) and DIEA (116 mg, 0.89 mmol), the resulting mixture was stirred overnight at room temperature, purified by prep-HPLC, lyophilized to afford 356 (179 mg, 28% yield) as a white solid.

### Example 251. Synthesis of tri-tert-butyl (24S, 28S)-1-(4-(((5S, 8S, 11S, 14S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-14-(4-(2, 5-dioxo-2, 5-dihydro-1H-pyrrol-1-yl) butanamido)-5, 8, 11-trimethyl-4, 7, 10, 13-tetraoxo-3, 6, 9, 12-tetraazaoctadecan-18-yl) carbamoyl)phenyl)-1, 12, 21, 26-tetraoxo-5, 8-dioxa-2, 11, 20, 25, 27-pentaazatriacontane-24, 28, 30-tricarboxylate (357)

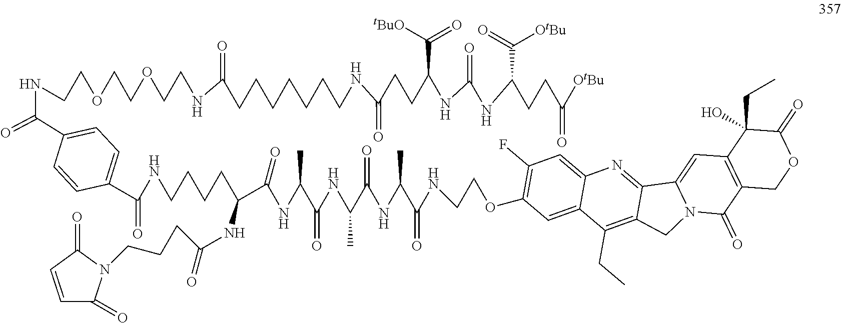 [SMILES: CCc1c2c(nc3cc(F)c(OCCNC(=O)[C@H](C)NC(=O)[C@H](C)NC(=O)[C@H](C)NC(=O)[C@H](CCCCNC(=O)c4ccc(C(=O)NCCOCCOCCNC(=O)CCCCCCCNC(=O)CC[C@H](NC(=O)N[C@@H](CCC(=O)OC(C)(C)C)C(=O)OC(C)(C)C)C(=O)OC(C)(C)C)cc4)NC(=O)CCCN4C(=O)C=CC4=O)cc13)-c1cc3c(c(=O)n1C2)COC(=O)[C@]3(O)CC]

To a stirred solution of 356 (40 mg, 0.028 mmol) in DMF (5 mL) at room temperature was added HATU (14 mg, 0.037 mmol), after stirring for 10 min., DIEA (8 mg, 0.057 mmol) and 128 (143 mg, 0.028 mmol) were added. The resulting mixture was stirred for 1 h, purified by prep-HPLC, and lyophilized to give 357 (40 mg, 76% yield) as a light yellow solid.

### Example 252. Synthesis of (24S, 28S)-1-(4-(((5S, 8S, 11S, 14S)-1-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)-14-(4-(2, 5-dioxo-2, 5-dihydro-1H-pyrrol-1-yl) butanamido)-5, 8, 11-trimethyl-4, 7, 10, 13-tetraoxo-3, 6, 9, 12-tetraazaoctadecan-18-yl) carbamoyl)phenyl)-1, 12, 21, 26-tetraoxo-5, 8-dioxa-2, 11, 20, 25, 27-pentaazatriacontane-24, 28, 30-tricarboxylic acid (358)

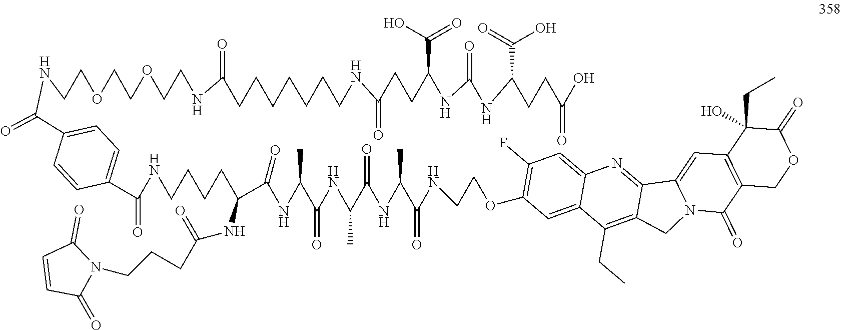 [SMILES: CCc1c2c(nc3cc(F)c(OCCNC(=O)[C@H](C)NC(=O)[C@H](C)NC(=O)[C@H](C)NC(=O)[C@H](CCCCNC(=O)c4ccc(C(=O)NCCOCCOCCNC(=O)CCCCCCCNC(=O)CC[C@H](NC(=O)N[C@@H](CCC(=O)O)C(=O)O)C(=O)O)cc4)NC(=O)CCCN4C(=O)C=CC4=O)cc13)-c1cc3c(c(=O)n1C2)COC(=O)[C@]3(O)CC]

To a stirred solution of 357 (40 mg, 0.022 mmol) in DCM (1 mL) at room temperature was added TFA (4 mL), and the reaction mixture was stirred at room temperature for 2 h, purified by prep-HPLC, lyophilized to afford 358 (19 mg, 52% yield) as a white solid.

### Example 253. Synthesis of tri-tert-butyl (24S, 28S)-1-(4-((5-(4-(2, 5-dioxo-2, 5-dihydro-1H-pyrrol-1-yl) butanamido)-6-((1-((1-(((S)-1-(((1R, 9R)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-1-oxopropan-2-yl)amino)-1-oxopropan-2-yl)amino)-1-oxopropan-2-yl)amino)-6-oxohexyl) carbamoyl)phenyl)-1, 12, 21, 26-tetraoxo-5, 8-dioxa-2, 11, 20, 25, 27-pentaazatriacontane-24, 28, 30-tricarboxylate (360)

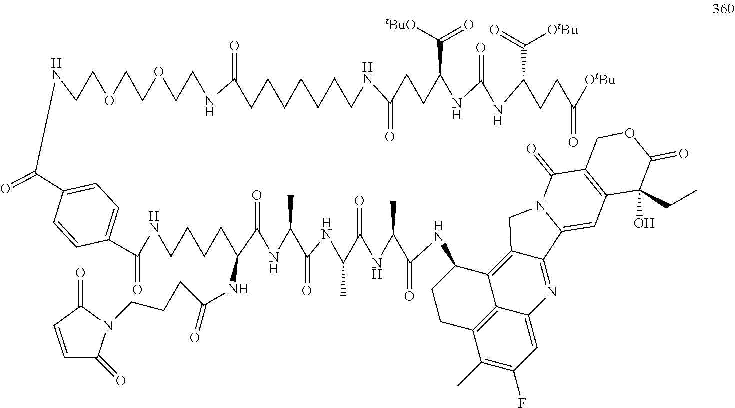 [SMILES: CC[C@]1(O)C(=O)OCc2c1cc1n(c2=O)Cc2c-1nc1cc(F)c(C)c3c1c2[C@H](NC(=O)[C@H](C)NC(=O)[C@H](C)NC(=O)[C@H](C)NC(=O)[C@H](CCCCNC(=O)c1ccc(C(=O)NCCOCCOCCNC(=O)CCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)CC3]

To a stirred solution of 356 (50 mg, 0.036 mmol) in DMF (3 mL) at room temperature was added HATU (21 mg, 0.053 mmol), after stirring for 10 min., DIEA (9 mg, 0.071 mmol) and 215 (19 mg, 0.035 mmol) were added. The resulting mixture was stirred for 1 h, purified by prep-HPLC, and lyophilized to give 360 (52 mg, 80% yield) as a white solid.

### Example 254. Synthesis of (24S, 28S)-1-(4-((5-(4-(2, 5-dioxo-2, 5-dihydro-IH-pyrrol-1-yl) butanamido)-6-((1-((1-(((S)-1-(((1R, 9R)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-1-oxopropan-2-yl)amino)-1-oxopropan-2-yl)amino)-1-oxopropan-2-yl)amino)-6-oxohexyl) carbamoyl)phenyl)-1, 12, 21, 26-tetraoxo-5, 8-dioxa-2, 11, 20, 25, 27-pentaazatriacontane-24, 28, 30-tricarboxylic acid (361)

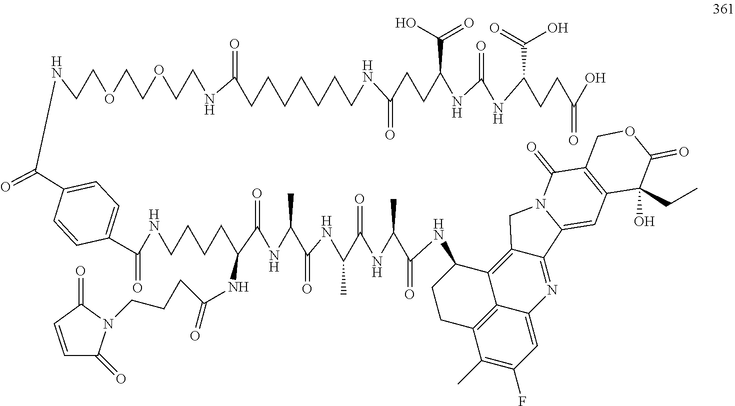 [SMILES: CC[C@]1(O)C(=O)OCc2c1cc1n(c2=O)Cc2c-1nc1cc(F)c(C)c3c1c2[C@H](NC(=O)[C@H](C)NC(=O)[C@H](C)NC(=O)[C@H](C)NC(=O)[C@H](CCCCNC(=O)c1ccc(C(=O)NCCOCCOCCNC(=O)CCCCCCCNC(=O)CC[C@H](NC(=O)N[C@@H](CCC(=O)O)C(=O)O)C(=O)O)cc1)NC(=O)CCCN1C(=O)C=CC1=O)CC3]

To a stirred solution of 360 (52 mg, 0.028 mmol) in DCM (1 mL) at room temperature was added TFA (4 mL), and the reaction mixture was stirred at room temperature for 2 h, purified by prep-HPLC, lyophilized to afford 361 (32 mg, 64% yield) as a white solid.

### Example 255. Synthesis of compound 364

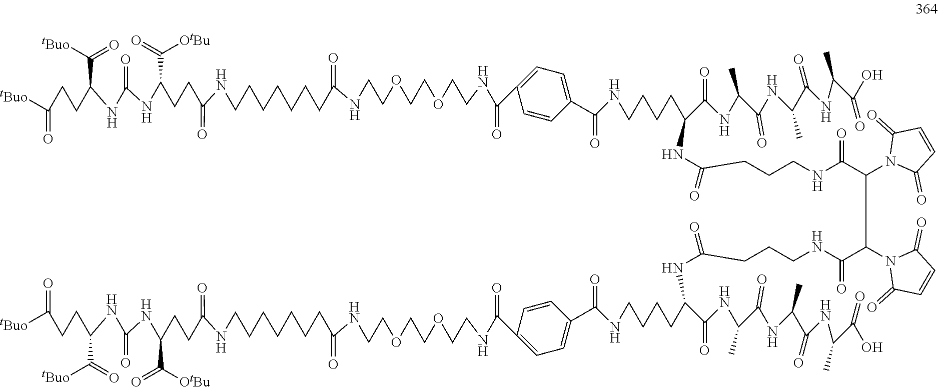

To a stirred solution of 363 (100 mg, 0.12 mmol) in DMF (5 mL) at room temperature were added 355 (300 mg, 0.24 mmol) and DIEA (72 mg, 0.55 mmol). The resulting mixture was stirred at room temperature for 2 h, purified by prep-HPLC, lyophilized to afford 364 (70 mg, 19% yield) as a white solid.

### Example 256. Synthesis of compound 365

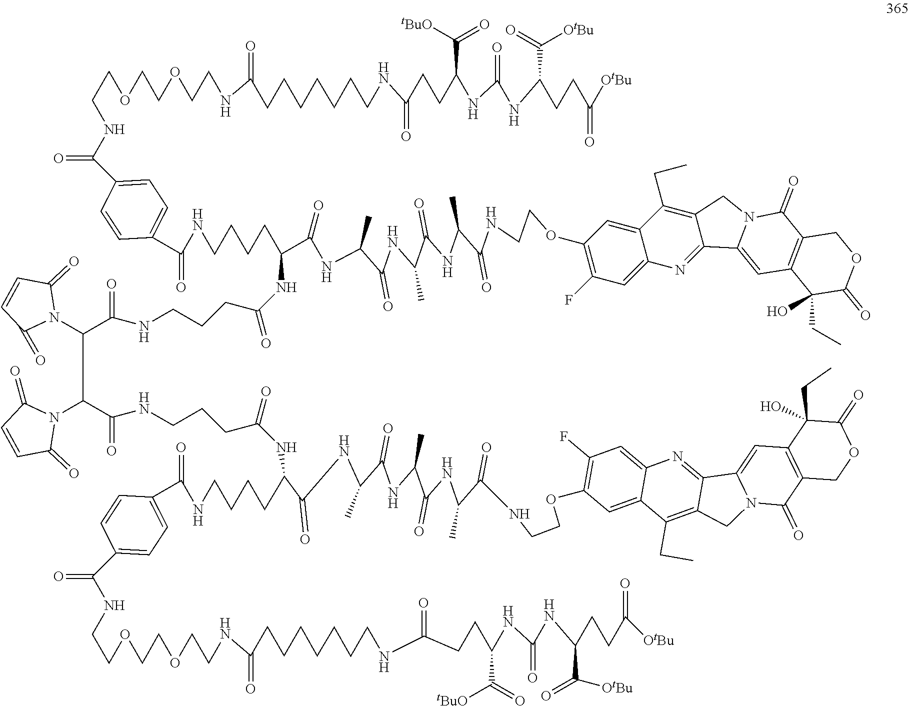

To a stirred solution of 364 (70 mg, 0.024 mmol) in DMF (3 mL) at room temperature was added HATU (23 mg, 0.059 mmol), and after stirring for 10 min., DIEA (14 mg, 0.10 mmol) and 128 (27 mg, 0.059 mmol) were added, the resulting mixture was stirred at room temperature for 2 h, purified by prep-HPLC, lyophilized to afford 365 (62 mg, 68% yield) as a white solid.

### Example 257. Synthesis of compound 366

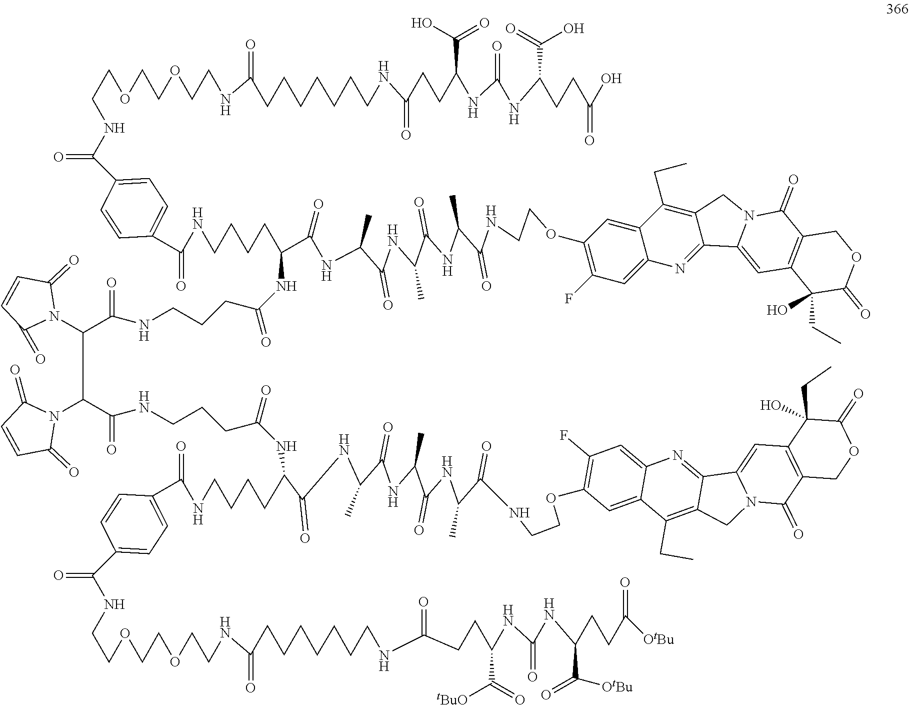

To a stirred solution of 365 (70 mg, 0.024 mmol) in DCM (1 mL) at room temperature was added TFA (6 mL), and the resulting mixture was stirred at room temperature for 2 h, concentrated, purified by prep-HPLC, lyophilized to afford 366 (27 mg, 4700 yield) as a white solid. ESI MS 3475.9 (M+H).

### Example 258. Synthesis of 1-(4-(tert-butoxycarbonyl)phenyl)-1-oxo-5, 8, 11, 14, 17, 20, 23, 26-octaoxa-2-azanonacosan-29-oic acid (368)

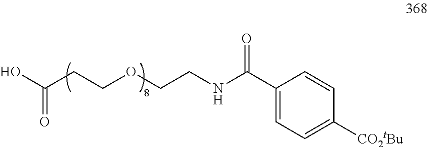

To a stirred solution of 1-amino-3, 6, 9, 12, 15, 18, 21, 24-octaoxaheptacosan-27-oic acid (4.80 g, 10.8 mmol) in water (20 mL) at 0° C. were added sodium hydroxide (0.43 g, 10.8 mmol) and sodium bicarbonate (1.14 g, 10.8 mmol), followed by 5 (1.30 g, 5.4 mmol) in THF (10 mL) dropwise. The resulting mixture was stirred at 0° C. for 3 h, diluted with ethyl acetate (10 mL) and water (10 mL). The aqueous layer was separated and washed with ethyl acetate (10 mL), acidified by 1M HCl (10 mL), extracted with DCM (40 mL) for 2 times. The organic layers were washed with brine (20 mL), dried over anhydrous sodium sulfate, filtered, concentrated to afford 368 (4.88 g, 70% yield) as a brown oil.

### Example 259. Synthesis of bezyl tert-butyl ((S)-6-(((S)-1-(((S)-1-((2-(((S)-41,11-diethyl-8-fluoro-4-hydroxy-3, 14-dioxo-3, 4, 12, 14-tetrahydro-1H-pyrano[3′ 4:6, 7]indolizino[1, 2-b]quinolin-9-yl)oxy)ethyl)amino)-1-oxopropan-2-yl)amino)-3-methyl-1-oxobutan-2-yl)amino)-6-oxohexane-1, 5-diyl)dicarbamate (369)

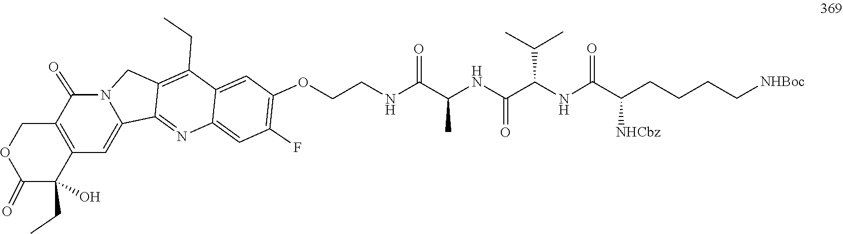 [SMILES: CCc1c2c(nc3cc(F)c(OCCNC(=O)[C@H](C)NC(=O)[C@@H](NC(=O)[C@H](CCCCNC(=O)OC(C)(C)C)NC(=O)OCc4ccccc4)C(C)C)cc13)-c1cc3c(c(=O)n1C2)COC(=O)[C@]3(O)CC]

To a solution of compound 131 (3.6 g, 5.8 mmol) and 1 (2.6 g, 6.9 mmol) in DMF (40 mL) were added HATU (3.3 g, 8.7 mmol) and DIEA (2.3 g, 17.5 mmol) at 0° C. The mixture was stirred at 0° C. for 2 h, quenched with water (200 mL). The formed precipitate was collected by filtration. The filter cake was purified by flash column chromatography to give 369 (2.80 g, 49% yield) as a yellow solid. ESI-MS m/z: [M+H]⁺ calcd. for C₅₁H₆₄FN₇O₁₂, 986.46; found: 986.55.

### Example 260. Synthesis of benzyl ((S)-6-amino-1-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl)amino)-1-oxohexan-2-yl) carbamate (370)

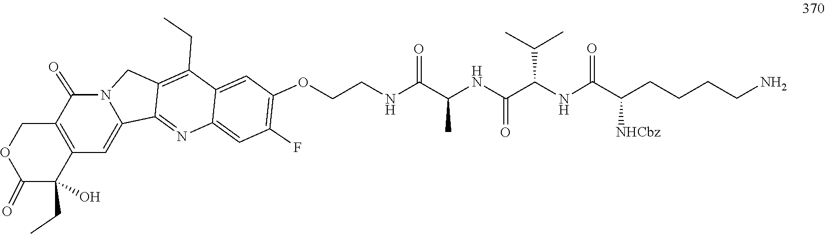 [SMILES: CCc1c2c(nc3cc(F)c(OCCNC(=O)[C@H](C)NC(=O)[C@@H](NC(=O)[C@H](CCCCN)NC(=O)OCc4ccccc4)C(C)C)cc13)-c1cc3c(c(=O)n1C2)COC(=O)[C@]3(O)CC]

A mixture of compound 369 (2.8 g, 2.8 mmol) in DCM (20 mL) and TFA (10 mL) was stirred at 30° C. for 2 h. LC-MS showed the reaction was completed. The mixture was concentrated under vacuum, triturated with MBTE. After filtration, the filter cake was dried under vacuum to give compound 370 as a yellow solid (2.3 g, 92% yield). ESI-MS m/z: [M+H]⁺calcd. for C₄₆H₅₆FN₇O₁₀, 886.41; found: 886.52.

### Example 261. Synthesis of tert-butyl 4-(((33S, 36S, 39S)-33-(((benzyloxy) carbonyl)amino)-43-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-36-isopropyl-39-methyl-27, 34, 37, 40-tetraoxo-3, 6, 9, 12, 15, 18, 21, 24-octaoxa-28, 35, 38, 41-tetraazatritetracontyl) carbamoyl)benzoate (371)

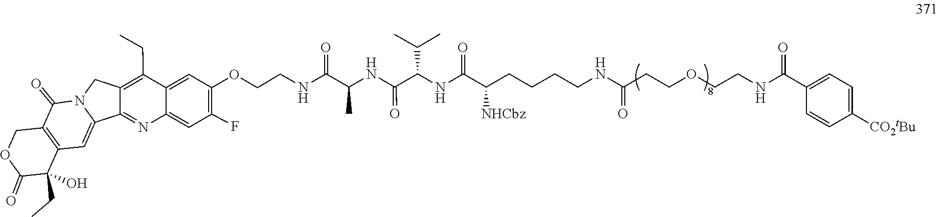

To a solution of compound 370 (2.5 g, 2.8 mmol) and compound 368 (2.0 g, 3.1 mmol) in DCM (80 mL) were added HATU (1.6 g, 4.2 mmol) and DIEA (3.7 g, 28.2 mmol) at 0° C. The mixture was stirred 0° C. for 2 h, washed with brine, dried over anhydrous sodium sulfate and concentrated. The residue was purified by flash column chromatography to give compound 371, as a yellow solid (2.0 g, 47% yield). ESI-MS m/z: [M+H]⁺calcd. for C₇₇H₁₀₅FN₈O₂₂, 1513.76; found: 1513.79.

### Example 262. Synthesis of 4-(((33S, 36S, 39S)-33-(((benzyloxy) carbonyl)amino)-43-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-36-isopropyl-39-methyl-27, 34, 37, 40-tetraoxo-3, 6, 9, 12, 15, 18, 21, 24-octaoxa-28, 35, 38, 41-tetraazatritetracontyl) carbamoyl)benzoic acid (372)

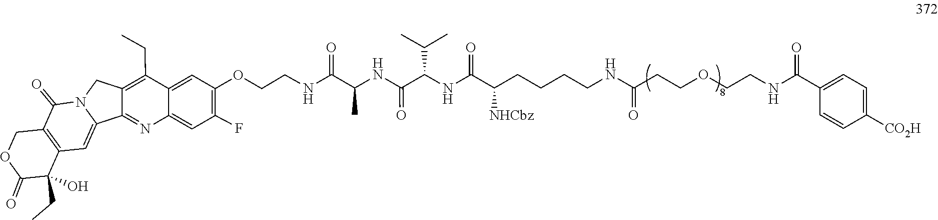

A mixture of compound 371 (2.0 g, 1.3 mmol) in DCM (20 mL) and TFA (5 mL) was stirred at 30° C. for 2 h. The mixture was concentrated under vacuum and purified by prep-HPLC to give compound 372, as a yellow solid (1.15 g, 59.6% yield). ESI-MS m/z: [M+H]⁺ calcd. for C₇₃H₉₇FN₈O₂₂, 1457.67; found: 1457.67.

### Example 263. Synthesis of tri-tert-butyl (13S, 17S)-1-(4-(((33S, 36S, 39S)-33-(((benzyloxy) carbonyl)amino)-43-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-36-isopropyl-39-methyl-27, 34, 37, 40-tetraoxo-3, 6, 9, 12, 15, 18, 21, 24-octaoxa-28, 35, 38, 41-tetraazatritetracontyl) carbamoyl)phenyl)-1, 10, 15-trioxo-2, 9, 14, 16-tetraazanonadecane-13, 17, 19-tricarboxylate (373)

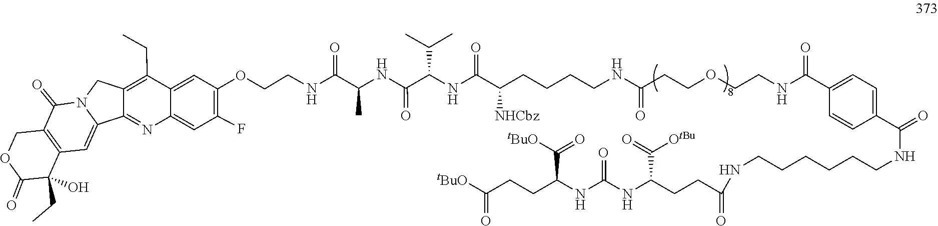

To a solution of compound 372 (1050 mg, 0.72 mmol) and compound 16 (425 mg, 0.72 mmol) in DMF (15 mL) were added HATU (410 mg, 1.1 mmol) and DIEA (280 mg, 2.2 mmol) at 0° C. The mixture was stirred at 0° C. for 2 h, purified by prep-HPLC to give compound 373, as a yellow solid (1.11 g, 70% yield). ESI-MS m/z: [M+H]⁺calcd. for C₁₀₂ H₁₄₉FN₁₂O2, 2026.25; found: 2026.35.

### Example 264. Synthesis of tri-tert-butyl (13S, 17S)-1-(4-(((33S, 36S, 39S)-33-amino-43-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-36-isopropyl-39-methyl-27, 34, 37, 40-tetraoxo-3, 6, 9, 12, 15, 18, 21, 24-octaoxa-28, 35, 38, 41-tetraazatritetracontyl) carbamoyl)phenyl)-1, 10, 15-trioxo-2, 9, 14, 16-tetraazanonadecane-13, 17, 19-tricarboxylate (374)

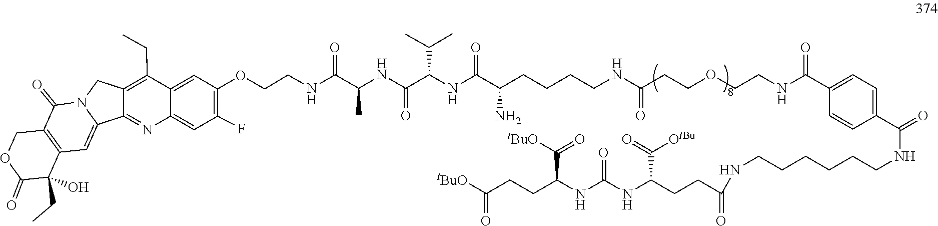

A mixture of compound 373 (1.11 g, 0.55 mmol) and Pd/C (0.2 g, 10 wt %) in isopropyl alcohol (20 mL) was stirred at 30° C. for 16 h under H₂. After filtration, the filtrate was concentrated to give compound 374, as an off-white solid (920 mg, 88.8% yield). ESI-MS m/z: [M+H]⁺calcd. for C₉₄H₁₄₃FN₁₂O₂₇, 1892.20; found: 1892.30.

### Example 265. Synthesis of (13S, 17S)-1-(4-(((33S, 36S, 39S)-33-amino-43-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-36-isopropyl-39-methyl-27, 34, 37, 40-tetraoxo-3, 6, 9, 12, 15, 18, 21, 24-octaoxa-28, 35, 38, 41-tetraazatritetracontyl) carbamoyl)phenyl)-1, 10, 15-trioxo-2, 9, 14, 16-tetraazanonadecane-13, 17, 19-tricarboxylic acid (375)

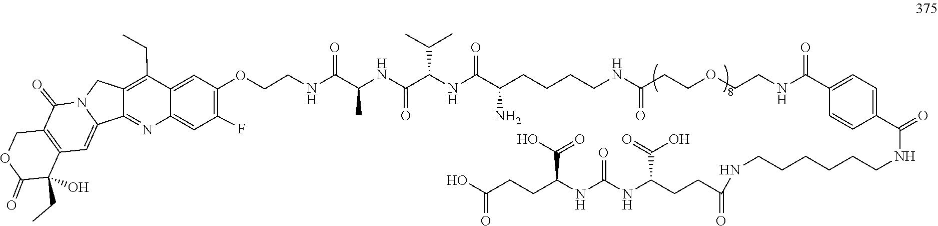

A mixture of compound 374 (700 mg, 0.37 mmol) in DCM (7 mL) and TFA (3 mL) was stirred at 30° C. for 2 h, and then concentrated under vacuum. The residue was triturated in MBTE, filtered to give compound 375, as a yellow solid (600 mg, 94% yield). ESI-MS m/z: [M+H]⁺ calcd. for C₈₂H₁₁₉FN₁₂O₂₇, 1723.83; found: 1723.89.

### Example 266. Synthesis of (13S, 17S)-1-(4-(((33S, 36S, 39S)-43-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-33-(4-(2, 5-dioxo-2, 5-dihydro-1H-pyrrol-1-yl) butanamido)-36-isopropyl-39-methyl-27, 34, 37, 40-tetraoxo-3, 6, 9, 12, 15, 18, 21, 24-octaoxa-28, 35, 38, 41-tetraazatritetracontyl) carbamoyl)phenyl)-1, 10, 15-trioxo-2, 9, 14, 16-tetraazanonadecane-13, 17, 19-tricarboxylic acid (376)

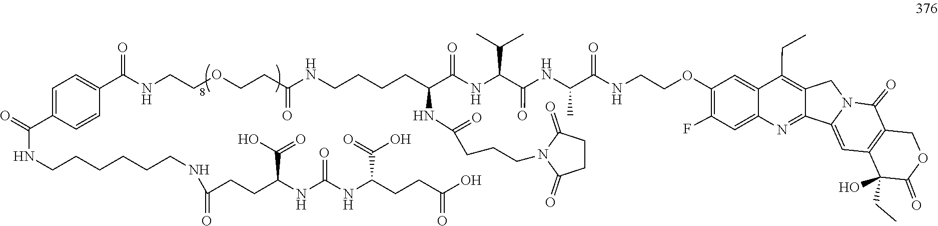

To a mixture of compound 375 (220 mg, 0.13 mmol) and compound 19 (40 mg, 0.14 mmol) in DMF (5 mL) was added DIEA (66 mg, 0.51 mmol) at 0° C. The mixture was stirred at 0° C. for 1 h and purified by prep-HPLC to give compound 376, as an off-white solid (107 mg, 44% yield). ESI-MS m/z: [M+H]⁺calcd. for C₉₀H₁₂₆FN₁₃O₃₀, 1888.87; found: 1888.98.

### Example 267. Synthesis of tri-tert-butyl (13S, 17S)-1-(4-(((5S, 8S, 11S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11-(4-(2, 5-dioxo-2, 5-dihydro-1H-pyrrol-1-yl) butanamido)-8-isopropyl-5-methyl-4, 7, 10, 17-tetraoxo-20, 23-dioxa-3, 6, 9, 16-tetraazapentacosan-25-yl) carbamoyl)phenyl)-1, 10, 15-trioxo-2, 9, 14, 16-tetraazanonadecane-13, 17, 19-tricarboxylate (379)

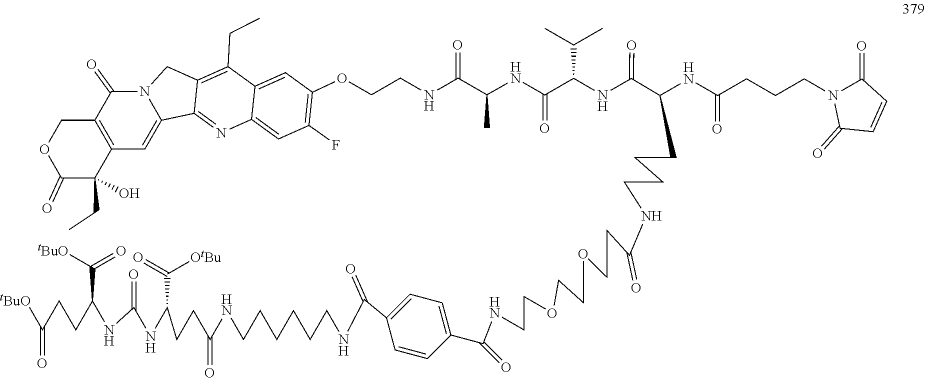 [SMILES: CCc1c2c(nc3cc(F)c(OCCNC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc4)NC(=O)CCCN4C(=O)C=CC4=O)C(C)C)cc13)-c1cc3c(c(=O)n1C2)COC(=O)[C@]3(O)CC]

To a stirred solution of 20 (220 mg, 0.171 mmol) and 378 (90 mg, 0.171 mmol) in DCM (10 mL) were added HATU (72 mg, 0.188 mmol) and DIEA (33 mg, 0.257 mmol). The mixture was stirred at room temperature for 1 h, and then concentrated under reduced pressure. The residue was purified by prep-HPLC to afford 379 (135 mg, 44% yield) as a white solid. ESI-MS m/z: calcd. for C₉₀H₁₂₆FN₁₃O₂₄ [M+H]+:1792.90; found 1793.02.

### Example 268. Synthesis of (13S, 17S)-1-(4-(((5S, 8S, 11S)-1-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)-11-(4-(2, 5-dioxo-2, 5-dihydro-1H-pyrrol-1-yl) butanamido)-8-isopropyl-5-methyl-4, 7, 10, 17-tetraoxo-20, 23-dioxa-3, 6, 9, 16-tetraazapentacosan-25-yl) carbamoyl)phenyl)-1, 10, 15-trioxo-2, 9, 14, 16-tetraazanonadecane-13, 17, 19-tricarboxylic acid (380)

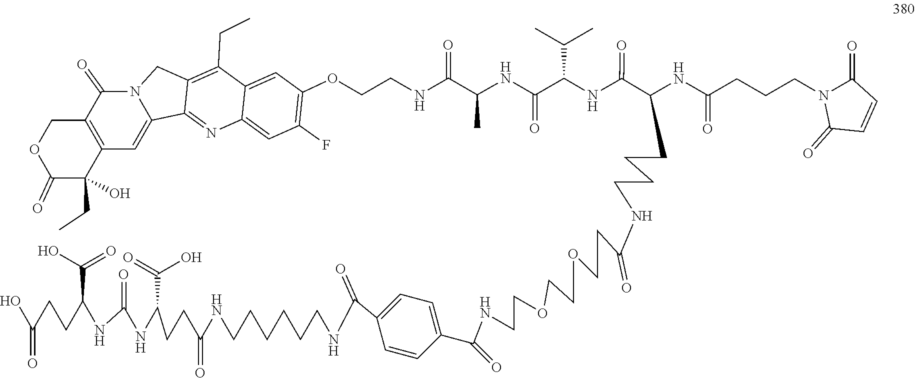 [SMILES: CCc1c2c(nc3cc(F)c(OCCNC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)O)C(=O)O)C(=O)O)cc4)NC(=O)CCCN4C(=O)C=CC4=O)C(C)C)cc13)-c1cc3c(c(=O)n1C2)COC(=O)[C@]3(O)CC]

To a stirred solution of 379 (135 mg, 0.075 mmol) in DCM (2 mL) was added TFA (10 mL). The mixture was stirred at room temperature for 1 h, concentrated under reduced pressure, purified by prep-HPLC to afford 380 (66 mg, 54% o yield) as a white solid. ESI-MS m/z: calcd. for C₇₈H₁₀₂FN₁₃024 [M+H]⁺:1624.71; found 1625.29.

### Example 269. Synthesis of hexa-tert-butyl 1, 1′-((15-((S)-5-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl)amino)-4-(4-(2, 5-dioxo-2, 5-dihydro-1H-pyrrol-1-yl) butanamido)-5-oxopentanoyl)-11, 19-dioxo-5, 8, 22, 25-tetraoxa-2, 12, 15, 18, 28-pentaazanonacosanedioyl)bis (4, 1-phenylene)) (13S, 13'S, 17S, 17'S)-bis (1, 10, 15-trioxo-2, 9, 14, 16-tetraazanonadecane-13, 17, 19-tricarboxylate) (382)

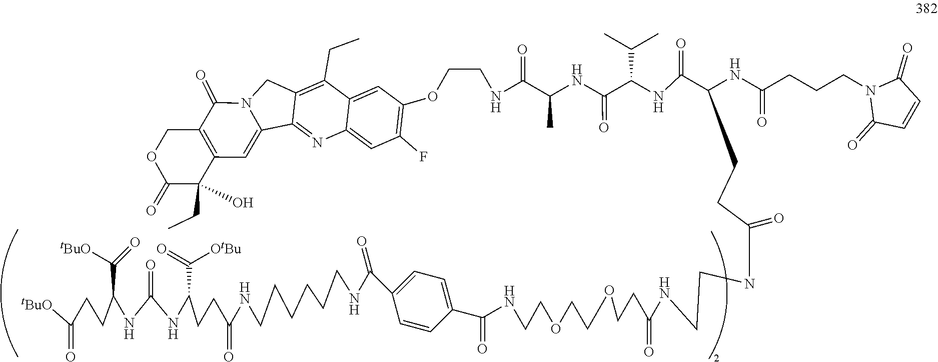

To a stirred solution of 69 (80.0 mg, 0.034 mmol) in DMF (5 mL) at room temperature were added 128 (16.0 mg, 0.038 mmol) and DIEA (7.0 mg, 0.052 mmol), the reaction mixture was stirred at room temperature for 0.5 h, purified by prep-HPLC, concentrated to afford 382 (70 mg, 74% yield) as a yellow solid.

### Example 270. Synthesis of (13S, 13'S, 17S, 17'S)-1, 1′-((15-((S)-5-(((S)-1-(((S)-1-((2-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)ethyl)amino)-1-oxopropan-2-yl)amino)-3-methyl-1-oxobutan-2-yl)amino)-4-(4-(2, 5-dioxo-2, 5-dihydro-1H-pyrrol-1-yl) butanamido)-5-oxopentanoyl)-11, 19-dioxo-5, 8, 22, 25-tetraoxa-2, 12, 15, 18, 28-pentaazanonacosanedioyl)bis (4, 1-phenylene))bis (1, 10, 15-trioxo-2, 9, 14, 16-tetraazanonadecane-13, 17, 19-tricarboxylic acid) (383)

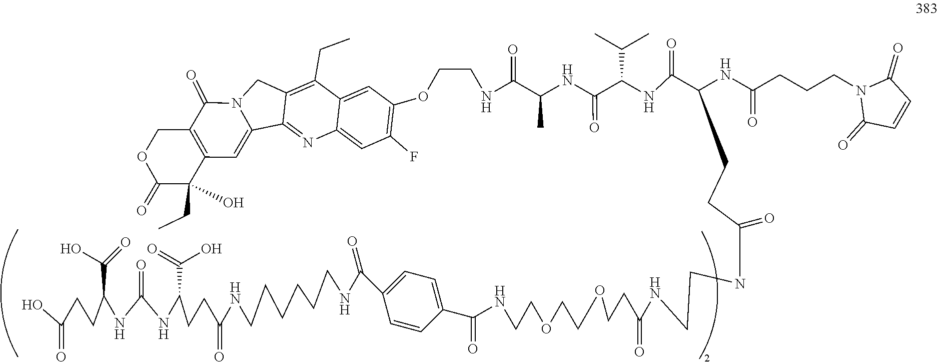

To a stirred solution of 382 (70.0 mg, 0.025 mmol) in DCM (5 mL) at room temperature was added TFA (6 mL), and the reaction was stirred for 3 h, concentrated, purified by prep-HPLC, lyophilized to afford 383 (12 mg, 19% yield) as a white solid.

### Example 271. Synthesis of 1-(4-((17S, 20S, 23S)-1-(4-(((7S, 11S)-7, 11-bis(tert-butoxycarbonyl)-2, 2-dimethyl-4, 9, 17-trioxo-3-oxa-8, 10, 16-triazahenicosan-21-yl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (385)

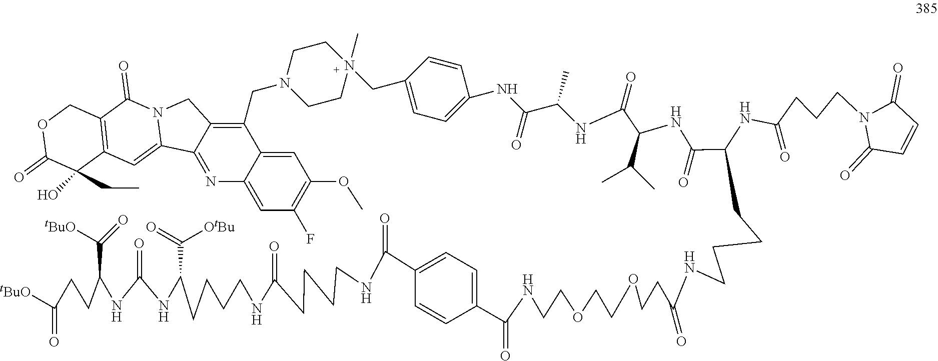

To a mixture of compound 185 (100 mg, 77.7 umol) and compound 40 (70 mg, 101 umol) in DMF (5 mL) were added HATU (44.5 mg, 116.6 umol) and DIEA (30 mg, 233.2 umol). The mixture was stirred at room temperature for 2 h, concentrated under vacuum. The residue was purified by prep-HPLC to give compound 385, as a brown oil (100 mg, 66% yield). ESI-MS m/z: [M]⁺ calcd. for C₁₀₀H₁₃₉FN₁₅O₂₄, 1953.01; found: 1953.02.

### Example 272. Synthesis of 1-(4-((17S, 20S, 23S)-1-(4-((5-(((S)-5-carboxy-5-(3-((S)-1, 3-dicarboxypropyl) ureido) pentyl)amino)-5-oxopentyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)benzyl)-4-(((S)-4-ethyl-8-fluoro-4-hydroxy-9-methoxy-3, 14-dioxo-3, 4, 12, 14-tetrahydro-1H-pyrano[3′, 4′:6, 7]indolizino[1, 2-b]quinolin-11-yl)methyl)-1-methylpiperazin-1-ium (386)

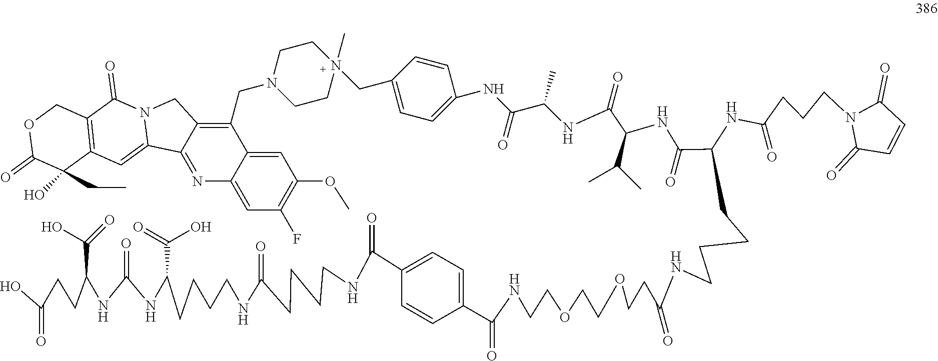

A mixture of compound 385 (100 mg, 51.2 umol) DCM (2 mL) and TFA (1 mL) was stirred at room temperature for 2 h. LC-MS showed the reaction was completed. The mixture was concentrated under vacuum and the residue was purified by prep-HPLC to give compound 386, as a yellow solid (10.0 mg, 11% yield). ESI-MS m/z: [M] calcd. for Cs₈H₁₁₅FN₁₅O₂₄, 1784.8; found:1784.8.

### Example 273. Synthesis of tri-tert-butyl (13S, 17S)-1-(4-(((5S, 8S, 11S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 7, 15-trioxo-2, 8, 14, 16-tetraazanonadecane-13, 17, 19-tricarboxylate (389)

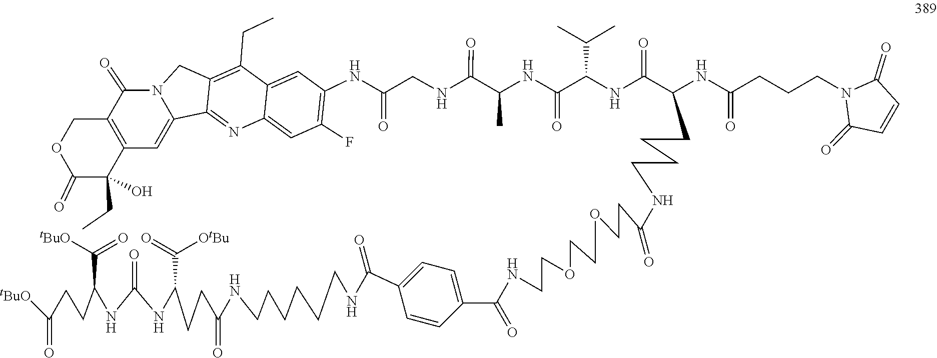 [SMILES: CCc1c2c(nc3cc(F)c(NC(=O)CNC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc4)NC(=O)CCCN4C(=O)C=CC4=O)C(C)C)cc13)-c1cc3c(c(=O)n1C2)COC(=O)[C@]3(O)CC]

To a mixture of compound 40 (110 mg, 85.5umol) and compound 388 (46 mg, 85.5umol) in DMF (5 mL) were added HATU (49 mg, 128.3umol) and DIEA (17 mg, 85.9umol). The mixture was stirred at room temperature for 2 h. LC-MS showed the reaction was completed. The mixture was concentrated under vacuum and the residue was purified by prep-HPLC to give compound 389, as a brown oil (40 mg, 26% yield). ESI-MS m/z: [M+H]⁺calcd. for C₉₀H₁₂₅FN₁₄O₂₄, 1805.90; found: 1805.92.

### Example 274. Synthesis of (13S, 17S)-1-(4-(((5S, 8S, 11S)-1-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)amino)-11-(4-(2, 5-dioxo-2, 5-dihydro-1H-pyrrol-1-yl) butanamido)-8-isopropyl-5-methyl-1, 4, 7, 10, 17-pentaoxo-20, 23-dioxa-3, 6, 9, 16-tetraazapentacosan-25-yl) carbamoyl)phenyl)-1, 7, 15-trioxo-2, 8, 14, 16-tetraazanonadecane-13, 17, 19-tricarboxylic acid (390)

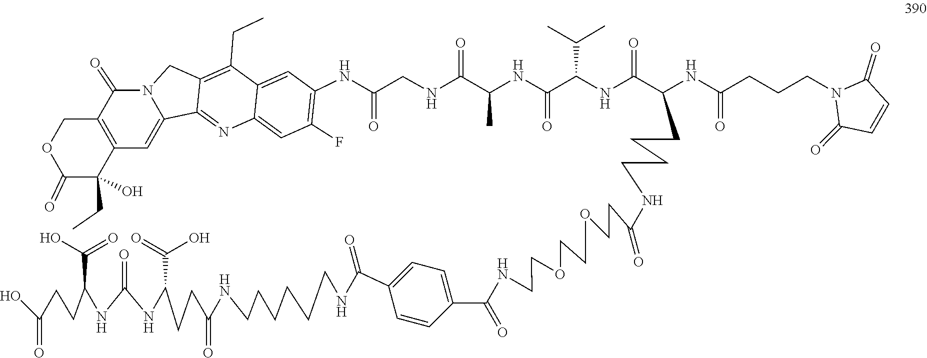 [SMILES: CCc1c2c(nc3cc(F)c(NC(=O)CNC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)O)C(=O)O)C(=O)O)cc4)NC(=O)CCCN4C(=O)C=CC4=O)C(C)C)cc13)-c1cc3c(c(=O)n1C2)COC(=O)[C@]3(O)CC]

A mixture of compound 389 (40 mg, 22.1 umol) DCM (2 mL) and TFA (1 mL) was stirred at room temperature for 2 h. LC-MS showed the reaction was completed. The mixture was concentrated under vacuum and the residue was purified by prep-HPLC to give compound 390, as a yellow solid (18.0 mg, 50% yield). ESI-MS m/z: [M+H]⁺calcd. for C₇₈H₁₀₁FN₁₄O₂₄, 1637.71; found:1637.72.

### Example 275. Synthesis of tri-tert-butyl (13S, 17S)-1-(4-(((15S, 18S, 21S)-22-((4-((((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)methyl)phenyl)amino)-15-(4-(2, 5-dioxo-2, 5-dihydro-1H-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylate (392)

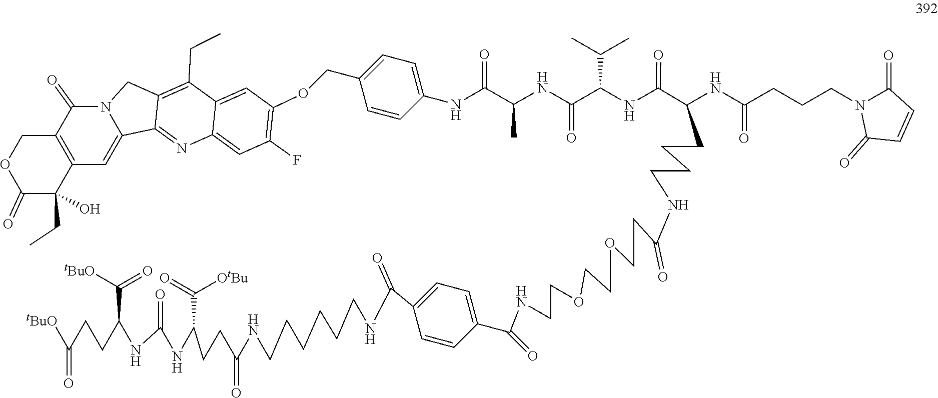 [SMILES: CCc1c2c(nc3cc(F)c(OCc4ccc(NC(=O)[C@H](C)NC(=O)[C@@H](NC(=O)[C@H](CCCCNC(=O)CCOCCOCCNC(=O)c5ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc5)NC(=O)CCCN5C(=O)C=CC5=O)C(C)C)cc4)cc13)-c1cc3c(c(=O)n1C2)COC(=O)[C@]3(O)CC]

To a mixture of compound 145 (150 mg, 237.8 μmol) and 40 (310 mg, 237.8 μmol) in DMF (4 mL) were added HATU (140 mg, 356.8 μmol) and DIEA (93 mg, 713.5 μmol). The mixture was stirred at 0° C. for 2 h, concentrated under vacuum. The residue was purified by flash column chromatography to give compound 392, as a brown solid (270 mg, 50.3% yield).

### Example 276. Synthesis of (13S, 17S)-1-(4-(((15S, 18S, 21S)-22-((4-((((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)methyl)phenyl)amino)-15-(4-(2, 5-dioxo-2, 5-dihydro-IH-pyrrol-1-yl) butanamido)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylic acid (393)

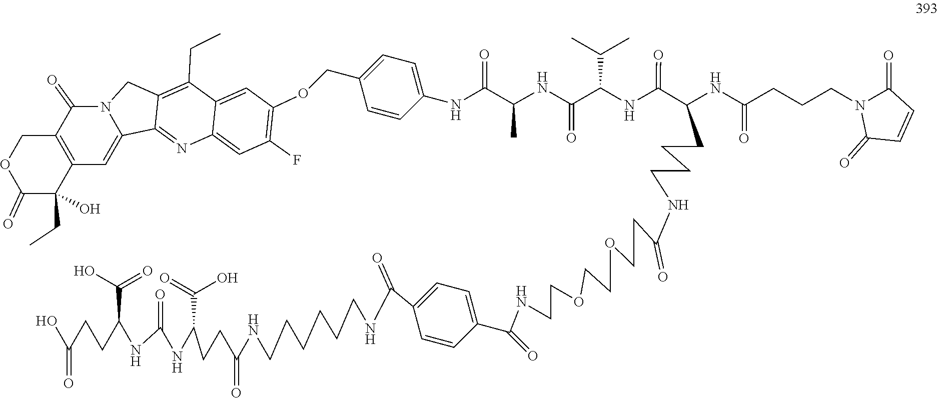 [SMILES: CCc1c2c(nc3cc(F)c(OCc4ccc(NC(=O)[C@H](C)NC(=O)[C@@H](NC(=O)[C@H](CCCCNC(=O)CCOCCOCCNC(=O)c5ccc(C(=O)NCCCCCCNC(=O)CC[C@H](NC(=O)N[C@@H](CCC(=O)O)C(=O)O)C(=O)O)cc5)NC(=O)CCCN5C(=O)C=CC5=O)C(C)C)cc4)cc13)-c1cc3c(c(=O)n1C2)COC(=O)[C@]3(O)CC]

A mixture of compound 392 (260 mg, 136.9 umol) in DCM (2 mL) and TFA (1 mL) was stirred at room temperature for 2 h, and concentrated under vacuum. The residue was purified by prep-HPLC to give 393, as an off-white solid (40 mg, 17% yield).

### Example 277. Synthesis of tri-tert-butyl (13S, 17S)-1-(4-(((15S, 18S, 21R)-15-(4-(2, 5-dioxo-2, 5-dihydro-IH-pyrrol-1-yl) butanamido)-22-(((1R, 9R)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylate (395)

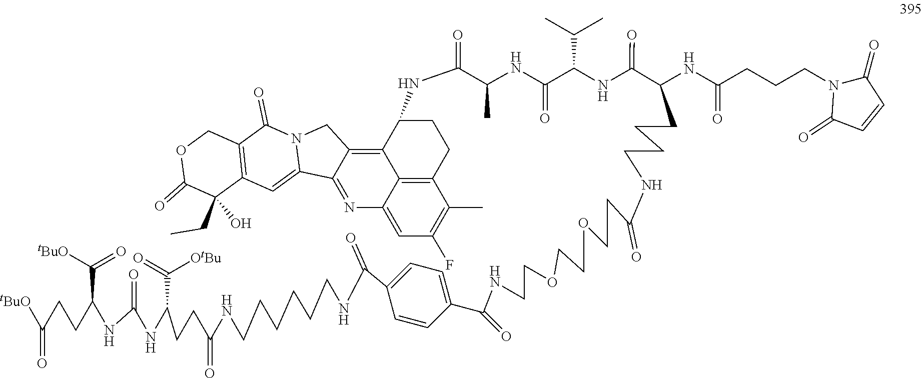 [SMILES: CC[C@@]1(O)C(=O)OCc2c1cc1n(c2=O)Cc2c-1nc1cc(F)c(C)c3c1c2[C@H](NC(=O)[C@H](C)NC(=O)[C@@H](NC(=O)[C@H](CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(C)C)CC3]

To a stirred solution of 40 (200 mg, 0.155 mmol, 1.00 eq) in DMF (4 mL) at room temperature were added HATU (62.07 mg, 0.163 mmol, 1.05 eq), 206 (106 mg, 0.171 mmol, 1.10 eq) and DIEA (68 mL, 0.389 mmol, 2.50 eq) in sequence. The resulting mixture was stirred at room temperature for 1 h, concentrated in vacuo. Compound 395 was obtained as a yellow oil (276 mg, 100% yield), which was used without further purification. ESI-MS m/z: calcd. for C₉₀H₁₂₅FN₁₃O₂₃ [M+H]⁺ 1774.8990; found 1775.4086.

### Example 278. Synthesis of (13S, 17S)-1-(4-(((15S, 18S, 21R)-15-(4-(2, 5-dioxo-2, 5-dihydro-1H-pyrrol-1-yl) butanamido)-22-(((1R, 9R)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl)amino)-18-isopropyl-21-methyl-9, 16, 19, 22-tetraoxo-3, 6-dioxa-10, 17, 20-triazadocosyl) carbamoyl)phenyl)-1, 7, 15-trioxo-2, 8, 14, 16-tetraazanonadecane-13, 17, 19-tricarboxylic acid (396)

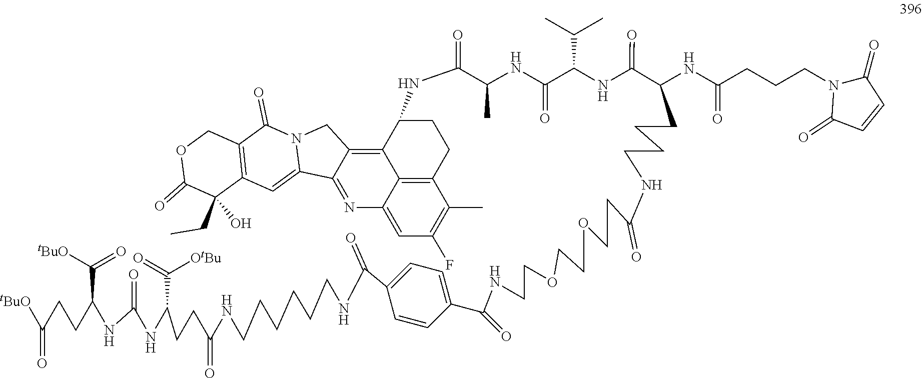 [SMILES: CC[C@@]1(O)C(=O)OCc2c1cc1n(c2=O)Cc2c-1nc1cc(F)c(C)c3c1c2[C@H](NC(=O)[C@H](C)NC(=O)[C@@H](NC(=O)[C@H](CCCCNC(=O)CCOCCOCCNC(=O)c1ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc1)NC(=O)CCCN1C(=O)C=CC1=O)C(C)C)CC3]

To a stirred solution of 395 (276 mg, 0.155 mmol) in DCM (3 mL) at room temperature was added TFA (3 mL). The resulting mixture was stirred at room temperature for 1 h, and concentrated in vacuo. Purification by prep-HPLC and lyophilization gave compound 396 as a yellow oil (24.0 mg, 9% yield). ESI-MS m/z: calcd. for C₇₈H₁₀₁FN₁₃O₂₃ [M+H]⁺ 1606.7112; found 1607.2284.

### Example 279. Synthesis of tri-tert-butyl (13S, 17S)-1-(4-(((5S, 8S, 11S)-1-(((S)-4, 11-diethyl-8-fluoro-4-hydroxy-3, 14-dioxo-3, 4, 12, 14-tetrahydro-IH-pyrano[3′, 4′:6, 7]indolizino[1, 2-b]quinolin-9-yl)oxy)-11-(4-(2, 5-dioxo-2, 5-dihydro-IH-pyrrol-1-yl) butanamido)-8-isopropyl-5-methyl-4, 7, 10, 17-tetraoxo-3, 20, 23-trioxa-6, 9, 16-triazapentacosan-25-yl) carbamoyl)phenyl)-1, 7, 15-trioxo-2, 8, 14, 16-tetraazanonadecane-13, 17, 19-tricarboxylate (399)

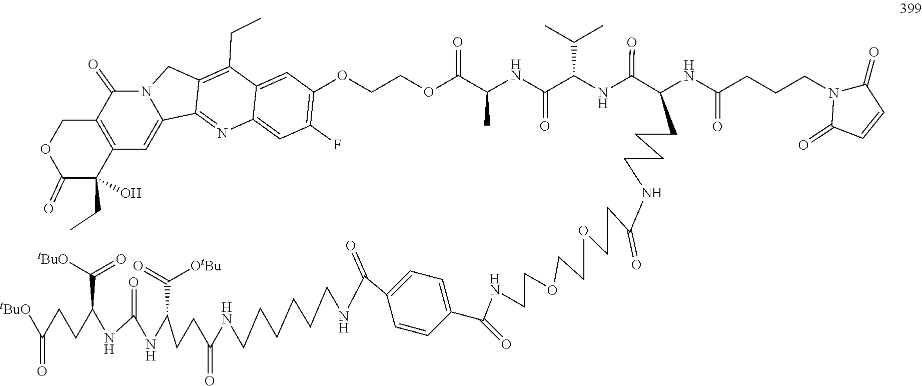 [SMILES: CCc1c2c(nc3cc(F)c(OCCOC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc4)NC(=O)CCCN4C(=O)C=CC4=O)C(C)C)cc13)-c1cc3c(c(=O)n1C2)COC(=O)[C@]3(O)CC]

To a stirred solution of 40 (280.4 mg, 0.218 mmol, 1.00 eq) in DMF (3 mL) at room temperature were added HATU (87.02 mg, 0.229 mmol, 1.05 eq), 398 (153.3 mg, 0.240 mmol, 1.10 eq) and DIEA (95 μL, 0.545 mmol, 2.50 eq) in sequence. The resulting mixture was stirred at room temperature for 1 h, and concentrated in vacuo. Compound 399 was obtained as a yellow oil (391 mg, 100% yield), which was used without further purification. ESI-MS m/z: calcd. for C₉₀H₁₂₆FN₁₂O₂₅ [M+H]⁺ 1793.8936; found 1794.2627.

### Example 280. Synthesis of (13S, 17S)-1-(4-(((5S, 8S, 11S)-1-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)-11-(4-(2, 5-dioxo-2, 5-dihydro-1H-pyrrol-1-yl) butanamido)-8-isopropyl-5-methyl-4, 7, 10, 17-tetraoxo-3, 20, 23-trioxa-6, 9, 16-triazapentacosan-25-yl) carbamoyl)phenyl)-1, 7, 15-trioxo-2, 8, 14, 16-tetraazanonadecane-13, 17, 19-tricarboxylic acid (400)

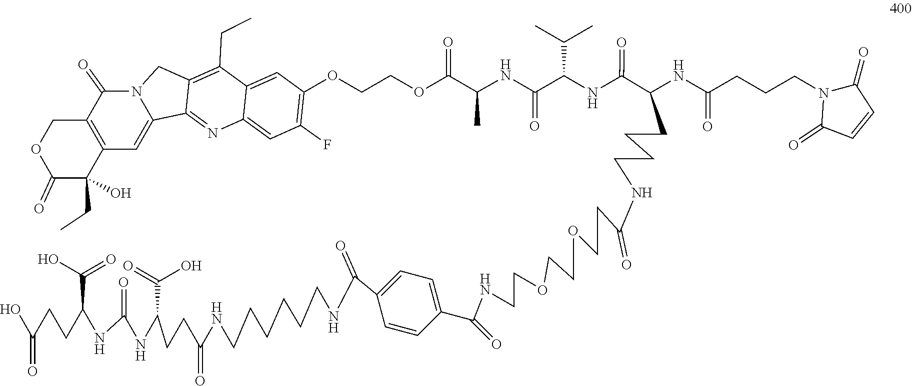 [SMILES: CCc1c2c(nc3cc(F)c(OCCOC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)O)C(=O)O)C(=O)O)cc4)NC(=O)CCCN4C(=O)C=CC4=O)C(C)C)cc13)-c1cc3c(c(=O)n1C2)COC(=O)[C@]3(O)CC]

To a stirred solution of 399 (391.0 mg, 0.218 mmol) in DCM (3 mL) at room temperature was added TFA (3 mL). The resulting mixture was stirred at room temperature for 1 h and concentrated in vacuo. Purification by prep-HPLC and lyophilization afforded product 400, as a yellow oil (38.0 mg, 10% yield). ESI-MS m/z: calcd. for C₇₈H₁₀₂FN₁₂O₂₅ [M+H]⁺ 1625.7058; found 1626.1152.

### Example 281. Synthesis of 1-(4-((17S, 20S, 23S)-1-(4-(((7S, 11S)-7, 11-bis(tert-butoxycarbonyl)-2, 2-dimethyl-4, 9, 17-trioxo-3-oxa-8, 10, 16-triazahenicosan-21-yl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (402)

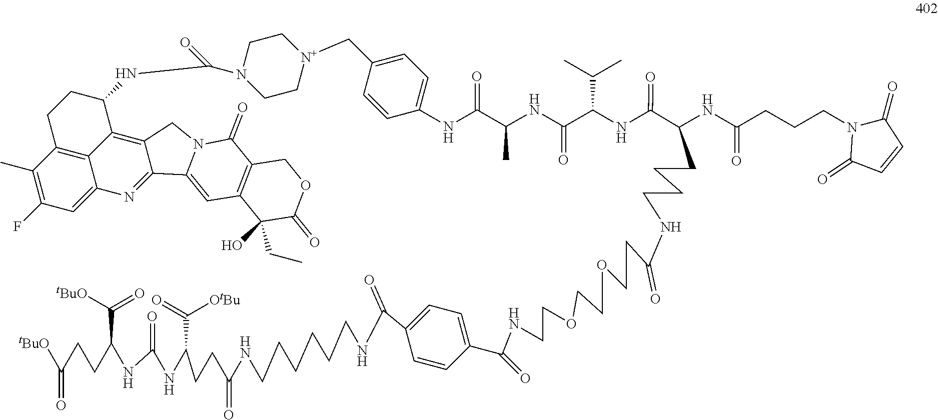 [SMILES: CC[C@@]1(O)C(=O)OCc2c1cc1n(c2=O)Cc2c-1nc1cc(F)c(C)c3c1c2[C@@H](NC(=O)N1CC[NH+](Cc2ccc(NC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)OC(C)(C)C)C(=O)OC(C)(C)C)C(=O)OC(C)(C)C)cc4)NC(=O)CCCN4C(=O)C=CC4=O)C(C)C)cc2)CC1)CC3]

To a solution of 40 (131 mg, 0.102 mmol, 1.0 eq) and 217 (87 mg, 0.102 mmol, 1.0 eq) in DMF (2 mL) were added HATU (46 mg, 0.122 mmol, 1.2 eq) and DIEA (39 mg, 0.305 mmol, 3.0 eq). The mixture was stirred at room temperature for 0.5 h, and then purified by prep-HPLC to afford 402 (110 mg, 54% yield) as a yellow solid. ESI-MS m/z: calcd. for C₁₀₃H₁₄₂FN₁₆024 [M]⁺:2006.0; found 2006.8.

### Example 282. Synthesis of 1-(4-((17S, 20S, 23S)-1-(4-((5-(((S)-5-carboxy-5-(3-((S)-1, 3-dicarboxypropyl) ureido) pentyl)amino)-5-oxopentyl) carbamoyl)phenyl)-17-(4-(2, 5-dioxo-2, 5-dihydro-1H-pyrrol-1-yl) butanamido)-20-isopropyl-23-methyl-1, 11, 18, 21-tetraoxo-5, 8-dioxa-2, 12, 19, 22-tetraazatetracosan-24-amido)benzyl)-4-(((1S, 9S)-9-ethyl-5-fluoro-9-hydroxy-4-methyl-10, 13-dioxo-2, 3, 9, 10, 13, 15-hexahydro-1H 12H-benzo[de]pyrano[3′, 4′:6, 7]indolizino[1, 2-b]quinolin-1-yl) carbamoyl)-1-methylpiperazin-1-ium (403)

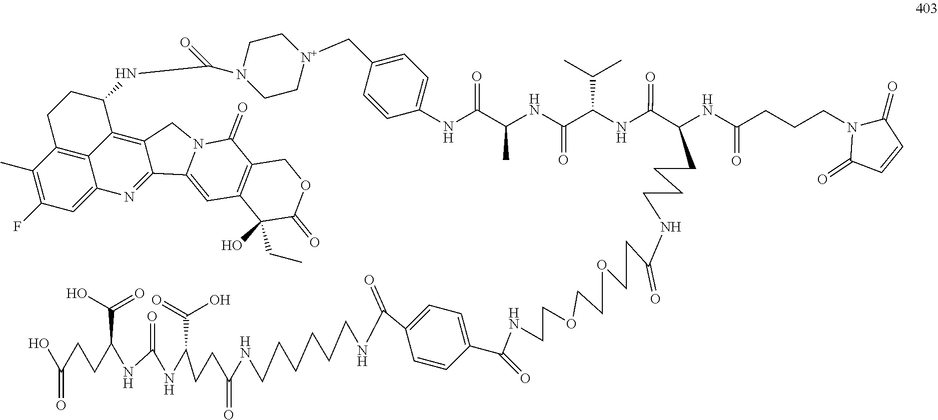 [SMILES: CC[C@@]1(O)C(=O)OCc2c1cc1n(c2=O)Cc2c-1nc1cc(F)c(C)c3c1c2[C@@H](NC(=O)N1CC[NH+](Cc2ccc(NC(=O)[C@H](C)NC(=O)[C@@H](NC(=O)[C@H](CCCCNC(=O)CCOCCOCCNC(=O)c4ccc(C(=O)NCCCCCCNC(=O)CC[C@H](NC(=O)N[C@@H](CCC(=O)O)C(=O)O)C(=O)O)cc4)NC(=O)CCCN4C(=O)C=CC4=O)C(C)C)cc2)CC1)CC3]

To a solution of 402 (110 mg, 0.055 mmol, 1.0 eq) in DCM (4 mL) was added TFA (5 mL). The mixture was stirred at room temperature for 2 h and then concentrated in vacuo. The residue was purified by prep-HPLC to afford 403 (37 mg, 37% yield) as a yellow solid. ESI-MS m/z: calcd. for C₉₁H₁₁₅FN₁₆O₂₄ [M]⁺:1837.9; found 1838.4.

### Example 283. Synthesis of (S)-5-(tert-butoxy)-2-(3-(2, 5-dioxo-2, 5-dihydro-1H-pyrrol-1-yl)propanamido)-5-oxopentanoic acid (405)

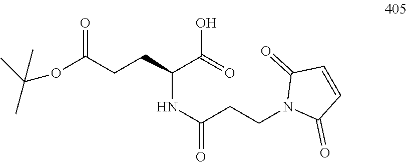 [SMILES: CC(C)(C)OC(=O)CC[C@H](NC(=O)CCN1C(=O)C=CC1=O)C(=O)O]

To a mixture of H-Glu (OtBu)—OH (2.03 g, 10.00 mmol) and compound 19 (2.93 g, 11.00 mmol) in DMF (50 mL) was added DIEA (2.59 g, 20.00 mmol) at r.t. The mixture was stirred at r.t. for 1 h. The mixture was purified by silica gel column, eluted with DCM and methanol, concentrated to give 2.87g of white solid (compound 405, 81.1% yield)

### Example 284. Synthesis of tert-butyl (S)-4-(4-(2, 5-dioxo-2, 5-dihydro-1H-pyrrol-1-yl)butanamido)-5-(((S)-1-(((R)-1-(((1S, 9S)-9-ethyl-5-fluoro-9-hydroxy-4-methyl-10, 13-dioxo-2, 3, 9, 10, 13, 15-hexahydro-1H, 12H-benzo[de]pyrano[3′, 4′:6, 7]indolizino[1, 2-b]quinolin-1-yl)amino)-1-oxopropan-2-yl)amino)-3-methyl-1-oxobutan-2-yl)amino)-5-oxopentanoate (406)

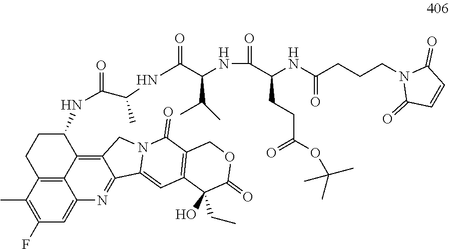 [SMILES: CC[C@@]1(O)C(=O)OCc2c1cc1n(c2=O)Cc2c-1nc1cc(F)c(C)c3c1c2[C@@H](NC(=O)[C@@H](C)NC(=O)[C@@H](NC(=O)[C@H](CCC(=O)OC(C)(C)C)NC(=O)CCCN1C(=O)C=CC1=O)C(C)C)CC3]

To a stirring solution of 208 (121 mg, 0.20 mmol) in DMF (1 mL) at room temperature was added 405 (71 mg, 0.20 mmol), HATU (91 mg, 0.24 mmol) and DIEA (65 mg, 0.50 mmol). The resulting mixture was stirred at room temperature for 30 min before it was concentrated in vacuo. HPLC reverse-phase purification and lyophilization afforded 406 (159 mg, yield 83%).

### Example 285. Synthesis of (S)-4-(4-(2, 5-dioxo-2, 5-dihydro-1H-pyrrol-1-yl)butanamido)-5-(((S)-1-(((R)-1-(((1S, 9S)-9-ethyl-5-fluoro-9-hydroxy-4-methyl-10, 13-dioxo-2, 3, 9, 10, 13, 15-hexahydro-1H, 12H-benzo[de]pyrano[3′, 4′:6, 7]indolizino[1, 2-b]quinolin-1-yl)amino)-1-oxopropan-2-yl)amino)-3-methyl-1-oxobutan-2-yl)amino)-5-oxopentanoic acid (407)

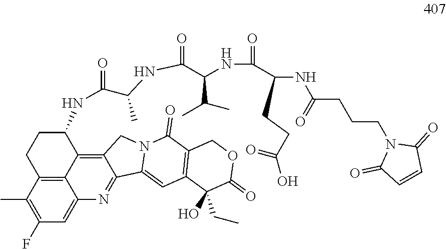 [SMILES: CC[C@@]1(O)C(=O)OCc2c1cc1n(c2=O)Cc2c-1nc1cc(F)c(C)c3c1c2[C@@H](NC(=O)[C@@H](C)NC(=O)[C@@H](NC(=O)[C@H](CCC(=O)O)NC(=O)CCCN1C(=O)C=CC1=O)C(C)C)CC3]

To a stirring solution of 406 (159 mg, 0.166 mmol) in DCM (3 mL) at room temperature was added TFA (3 mL). The reaction mixture was stirred for 1 h at room temperature, concentrated to afford 407 (149 mg, 100% yield).

### Example 286. Synthesis of tert-butyl (2-((2-((1-((2R, 4R, 5R)-4-((tert-butoxycarbonyl)oxy)-5-(((tert-butoxycarbonyl)oxy)methyl)-3, 3-difluorotetrahydrofuran-2-yl)-2-oxo-1, 2-dihydropyrimidin-4-yl)amino)-2-oxoethyl)amino)-2-oxoethyl)carbamate (409)

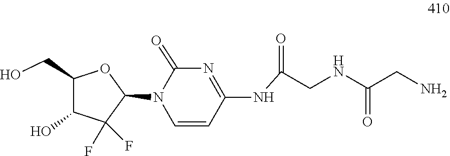 [SMILES: NCC(=O)NCC(=O)Nc1ccn([C@@H]2O[C@H](CO)[C@@H](O)C2(F)F)c(=O)n1]

To a stirring solution of 192 (250 mg, 0.539 mmol, 1.00 eq) and 408 (125 mg, 0.539 mmol, 1.00 eq) in acetonitrile (5 mL) at 0° C. were added NMI (88 mg, 1.078 mmol, 2.00 eq) and TCFH (151 mg, 0.539 mmol, 1.00 eq). The resulting mixture was stirred at room temperature for 1 h. The filtrate was concentrated in vacuo. Flash column chromatography (DCM: MeOH=95:5) afforded 409 (374 mg, 100%) as a white solid. HRMS (ESI): calcd for C₂₈H₄₁F₂N₅O₁2 [M+H]⁺ 678.2720, found 678.2720.

### Example 287. Synthesis of 2-amino-N-(2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)acetamide (410)

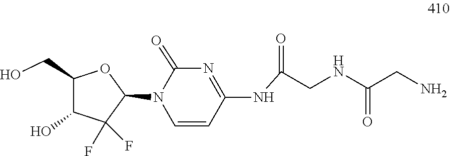 [SMILES: NCC(=O)NCC(=O)Nc1ccn([C@@H]2O[C@H](CO)[C@@H](O)C2(F)F)c(=O)n1]

To a stirring solution of 409 (374 mg, 0.552 mmol, 1.00eq) in DCM (8 mL) was added TFA (8 mL) at room temperature. The resulting mixture was stirred at room temperature for 1 h. The mixture was concentrated in vacuo. suspension was obtained by adding EA (20 mL), filtered, afford 410 (284 mg, 100%) as a white solid. HRMS (ESI): calcd for C₁₃H₁₇F₂N₅O₆[M+H]⁺ 378.1147, found 378.1147.

### Example 288. Synthesis of 2, 5-dioxopyrrolidin-1-yl 2, 2-dimethyl-4-oxo-3, 8, 11, 14, 17, 20, 23, 26, 29-nonaoxa-5-azadotriacontan-32-oate (412)

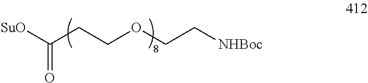

To a stirring solution of 411 (9.1 g, 16.77 mmol) in CH₂C₁₂ (100 mL) at room temperature was added EDCI (6.4 g, 33.53 mmol), NHS (2.9 g, 25.16 mmol), the reaction mixture was stirred for 1 h at room temperature, washed with water (100 mL), brine (100 mL), concentrated in vacuo. The residue was purified by silica gel column, eluted with ethyl acetate and petroleum ether, concentrated to afford 412 (9.0 g, 84% yield) as a colorless liquid.

### Example 289. Synthesis of 34, 34-bis((2-carboxyethoxy)methyl)-2, 2-dimethyl-4, 32-dioxo-3, 8, 11, 14, 17, 20, 23, 26, 29, 36-decaoxa-5, 33-diazanonatriacontan-39-oic acid (413)

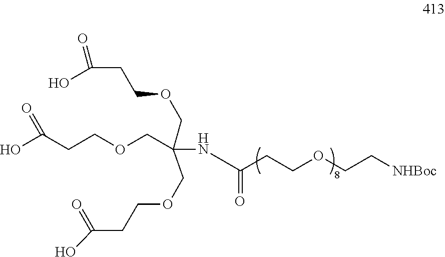

To a stirring solution of 197 (283 mg, 0.84 mmol) in DMF (5 mL) at room temperature were added 412 (536 mg, 0.84 mmol) and DIEA (254 mg, 2.00 mmol). The mixture was stirred for 2 h at room temperature. The reaction mixture was purified by pre-HPLC, lyophilized to afford 413 (492 mg, 68% yield) as a white solid.

### Example 290. Synthesis of tert-butyl (40-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-29, 29-bis ((3-((2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)amino)-3-oxopropoxy)methyl)-27, 34, 37, 40-tetraoxo-3, 6, 9, 12, 15, 18, 21, 24, 31-nonaoxa-28, 35, 38-triazatetracontyl)carbamate (414)

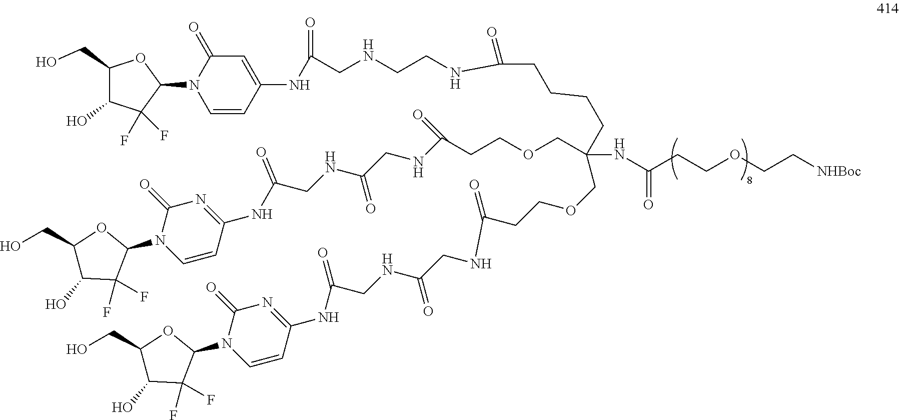

To a stirring solution of 413 (430 mg, 0.50 mmol) in DMF (10 mL) at room temperature were added 410 (566 mg, 1.50 mmol), HATU (697.0 mg, 1.83 mmol) and DIEA (355.0 mg, 2.74 mmol). The reaction mixture was stirred for 0.5 h at room temperature. The reaction mixture was diluted with CH₂C₁₂ (150 mL), washed with water (100 mL) concentrated, purified by silica gel column, eluted with CH₂Cl₂ and methanol, concentrated to afford 414 (679 mg, 70% yield) as a white solid.

### Example 291. Synthesis of 3, 3′-((2-(1-amino-3, 6, 9, 12, 15, 18, 21, 24-octaoxaheptacosan-27-amido)-2-((3-((2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)amino)-3-oxopropoxy)methyl)propane-1, 3-diyl)bis (oxy))bis (N-(2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)propanamide) (415)

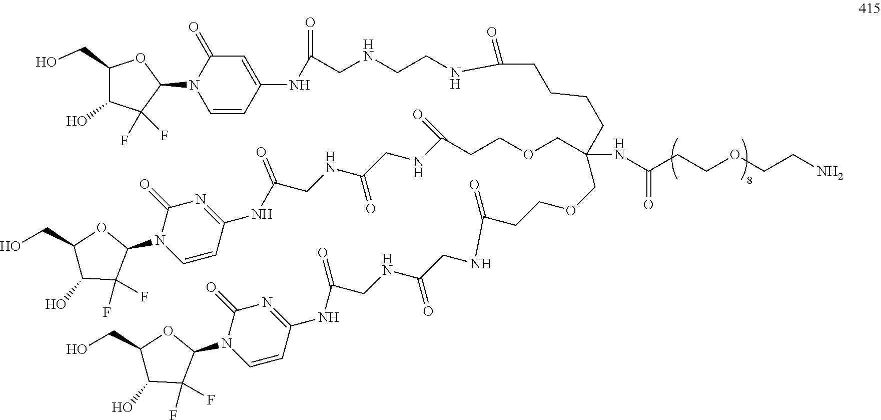

To a stirring solution of 414 (679 mg, 0.35 mmol, 1.00eq) in DCM (8 mL) was added TFA (8 mL) at room temperature. The resulting mixture was stirred at room temperature for 1 h. The mixture was concentrated in vacuo. suspension was obtained by adding EA (20 mL), filtered, afford 415 (634 mg, 100%) as a white solid.

### Example 292. Synthesis of compound 416

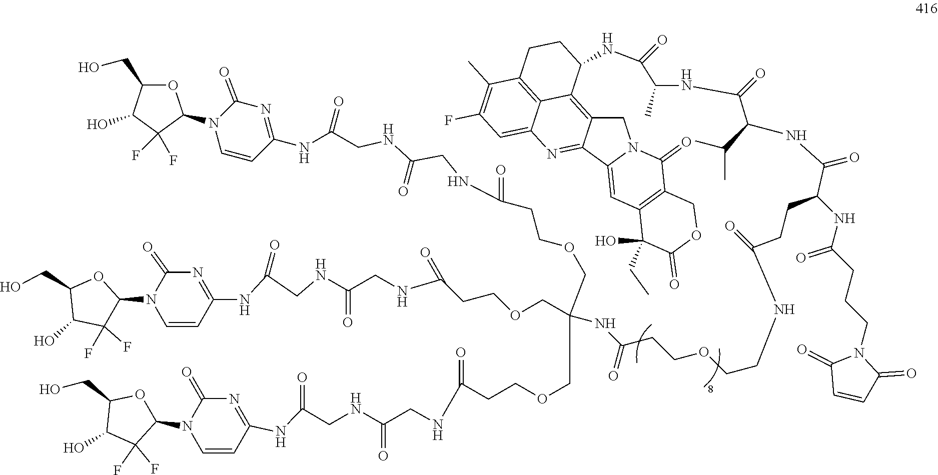

To a solution of 415 (184 mg, 0.102 mmol, 1.0 eq) and 407 (90 mg, 0.102 mmol, 1.0 eq) in DMF (2 mL) was added HATU (46 mg, 0.122 mmol, 1.2 eq), then added DIPEA (39 mg, 0.305 mmol, 3.0 eq). The mixture was stirred at RT for 0.5 h. The reaction mixture was purified by prep-HPLC to afford 416 (136 mg, 50% yield) as a yellow solid.

### Example 293. Synthesis of (2S)-N5-(2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)-2-((S)-37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-amido)-N1-((2S)-1-(((2R)-1-(((1R)-9-ethyl-5-fluoro-9-hydroxy-4-methyl-10, 13-dioxo-2, 3, 9, 10, 13, 15-hexahydro-1H, 12H-benzo[de]pyrano[3′, 4′:6, 7]indolizino[1, 2-b]quinolin-1-yl)amino)-1-oxopropan-2-yl)amino)-3-methyl-1-oxobutan-2-yl)pentanediamide 418

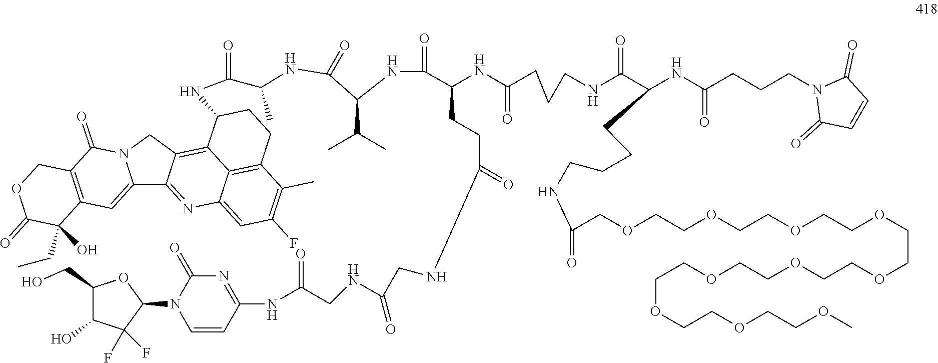 [SMILES: CC[C@]1(O)C(=O)OCc2c1cc1n(c2=O)Cc2c-1nc1cc(F)c(C)c3c1c2[C@H](NC(=O)[C@@H](C)NC(=O)[C@@H](NC(=O)[C@H](CCC(=O)NCC(=O)NCC(=O)Nc1ccn([C@@H]2O[C@H](CO)[C@@H](O)C2(F)F)c(=O)n1)NC(=O)CCCNC(=O)[C@H](CCCCNC(=O)COCCOCCOCCOCCOCCOCCOCCOCCOCCOC)NC(=O)CCCN1C(=O)C=CC1=O)C(C)C)CC3]

To a stirring solution of 210 (80.0 mg, 0.050 mmol) in DMF (1.5 mL) at room temperature were added HATU (30.42 mg, 0.08 mmol), 410 (28.55 mg, 0.076 mmol) and DIEA (19.32 mg, 0.15 mmol) in sequence. The resulting mixture was stirred at room temperature for 1 h before HPLC (MeCN: H2O (0.1% TFA)=42%) reverse-phase purification directly. Lyophilization afforded 418 (26.8 mg, 27%). HRMS (ESI): calcd for C₈₉H₁₂₅F₃N₁₅O₃₀ [M+H]⁺ 1941.04, found 1941.04

### Example 294. Synthesis of tert-butyl (4-((2-((1-((2R, 4R, 5R)-4-((tert-butoxycarbonyl)oxy)-5-(((tert-butoxycarbonyl)oxy)methyl)-3, 3-difluorotetrahydrofuran-2-yl)-2-oxo-1, 2-dihydropyrimidin-4-yl)amino)-2-oxoethyl)amino)-4-oxobutyl)carbamate (421)

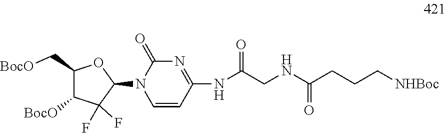

To a stirring solution of 192 (2 g, 4.3 mmol) in acetonitrile (30 mL) at 0° C. were added 420 (2.25 g, 8.6 mmol), TCFH (1.94 g, 6.9 mmol) and methylimidazole (1.06 g, 12.9 mmol). The reaction mixture was stirred for 3 h at room temperature, purified by pre-HPLC, lyophilized to afford 421 (1.52 g, 50% yield) as a white solid. ESI MS m/z: [M+H]⁺ calcd for C₃₀H₄₅F₂N₅O₁₂: 706.30; found 706.30.

### Example 295. Synthesis of 4-amino-N-(2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)butanamide (422

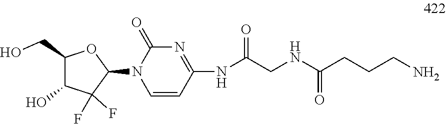 [SMILES: NCCCC(=O)NCC(=O)Nc1ccn([C@@H]2O[C@H](CO)[C@@H](O)C2(F)F)c(=O)n1]

To a stirring solution of 421 (1.5 g, 2 mmol) in CH₂C₁₂ (5 mL) at room temperature was added TFA (5 mL), the reaction mixture was stirred for 2 h at room temperature, concentrated to afford 422 (0.86 g, 100% yield) as a colorless liquid. ESI MS m/z: [M+H]⁺calcd for C₁₅H₂₁F₂N₅O₆: 406.15; found 406.15.

### Example 296. Synthesis of (2S)-N5-(4-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-4-oxobutyl)-2-((S)-37-(4-(2, 5-dioxo-2, 5-dihydro-1H-pyrrol-1-yl)butanamido)-31, 38-dioxo-2, 5, 8, 11, 14, 17, 20, 23, 26, 29-decaoxa-32, 39-diazatritetracontan-43-amido)-N1-((2S)-1-(((2R)-1-(((1R)-9-ethyl-5-fluoro-9-hydroxy-4-methyl-10, 13-dioxo-2, 3, 9, 10, 13, 15-hexahydro-1H, 12H-benzo[de]pyrano[3′, 4′:6, 7]indolizino[1, 2-b]quinolin-1-yl)amino)-1-oxopropan-2-yl)amino)-3-methyl-1-oxobutan-2-yl)pentanediamide (423)

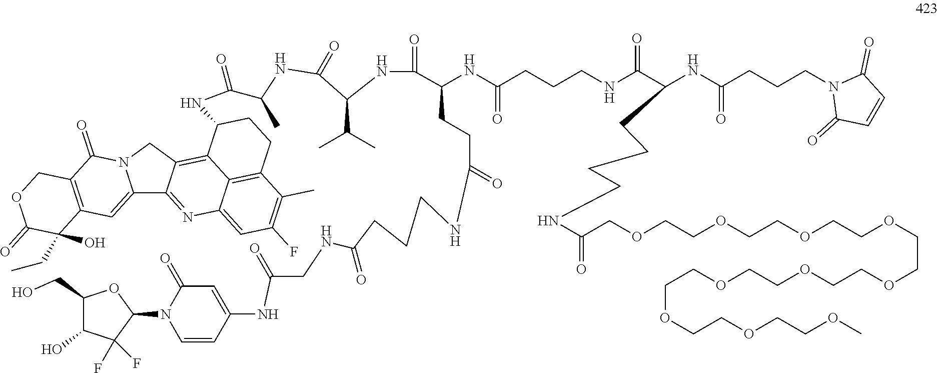 [SMILES: CC[C@]1(O)C(=O)OCc2c1cc1n(c2=O)Cc2c-1nc1cc(F)c(C)c3c1c2[C@H](NC(=O)[C@H](C)NC(=O)[C@@H](NC(=O)[C@H](CCC(=O)NCCCC(=O)NCC(=O)Nc1ccn([C@@H]2O[C@H](CO)[C@@H](O)C2(F)F)c(=O)c1)NC(=O)CCCNC(=O)[C@H](CCCCNC(=O)COCCOCCOCCOCCOCCOCCOCCOCCOCCOC)NC(=O)CCCN1C(=O)C=CC1=O)C(C)C)CC3]

To a stirring solution of 210 (200 mg, 0.13 mmol) in DMF (2 mL) at room temperature were added 422 (62 mg, 0.15 mmol), HATU (58 mg, 0.15 mmol) and DIEA (33 mg, 0.25 mmol). The reaction mixture was stirred for 20 minutes at room temperature, purified by pre-HPLC, lyophilized to afford 423 (158 mg, 63% yield) as a yellow solid. ESI MS m/z: [M+H]⁺ calcd for C₉₁H128F3N15030:1969.10; found 1970.10.

### Example 297. Synthesis of tert-butyl (1-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-17, 17-bis ((3-((4-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-4-oxobutyl)amino)-3-oxopropoxy)methyl)-1, 4, 9, 12, 19-pentaoxo-15-oxa-3, 8, 11, 18-tetraazadocosan-22-yl)carbamate (425)

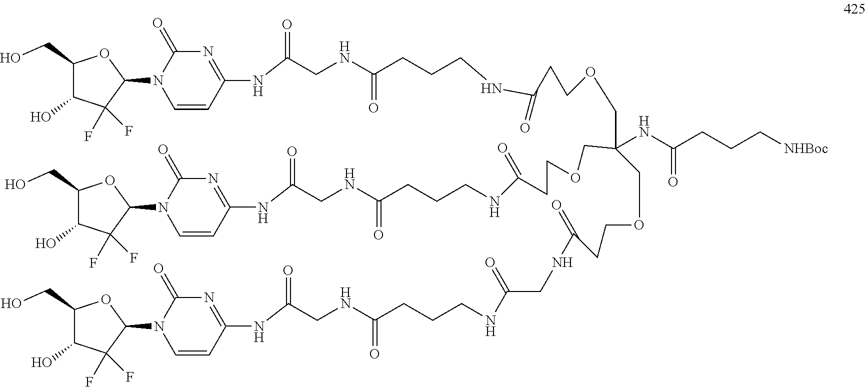 [SMILES: CC(C)(C)OC(=O)NCCCC(=O)NC(COCCC(=O)NCCCC(=O)NCC(=O)Nc1ccn([C@@H]2O[C@H](CO)[C@@H](O)C2(F)F)c(=O)n1)(COCCC(=O)NCCCC(=O)NCC(=O)Nc1ccn([C@@H]2O[C@H](CO)[C@@H](O)C2(F)F)c(=O)n1)COCCC(=O)NCC(=O)NCCCC(=O)NCC(=O)Nc1ccn([C@@H]2O[C@H](CO)[C@@H](O)C2(F)F)c(=O)n1]

To a stirring solution of 422 (219 mg, 0.54 mmol) in DMF (2 mL) at room temperature were added 199 (94 mg, 0.18 mmol), HATU (342 mg, 0.90 mmol) and DIEA (140 mg, 1.08 mmol). The reaction mixture was stirred for 40 minutes at room temperature, purified by pre-HPLC, lyophilized to afford 425 (84 mg, 28% yield). ESI MS m/z: [M+H]⁺ calcd for C₆₇H₉₅F₆N₁₇O₂₇:1685.58; found 1685.58.

### Example 298. Synthesis of 4, 4′-((17-(4-aminobutanamido)-1-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-1, 4, 9, 12-tetraoxo-17-((3-oxopropoxy)methyl)-15, 19-dioxa-3, 8, 11-triazadocosan-22-oyl)bis (azanediyl))bis(N-(2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)butanamide) (426)

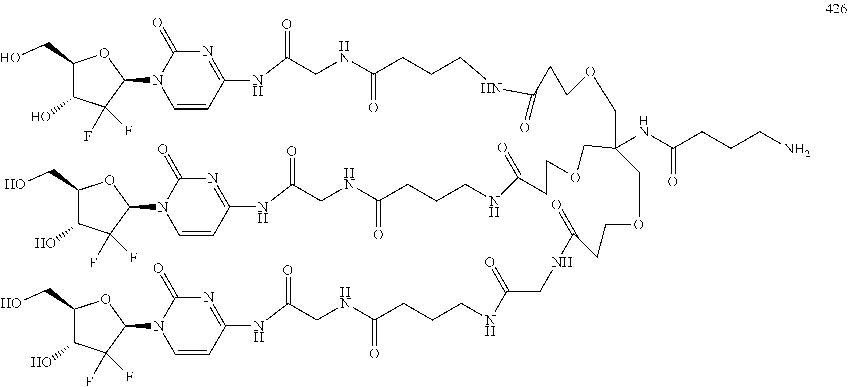 [SMILES: NCCCC(=O)NC(COCCC(=O)NCCCC(=O)NCC(=O)Nc1ccn([C@@H]2O[C@H](CO)[C@@H](O)C2(F)F)c(=O)n1)(COCCC(=O)NCCCC(=O)NCC(=O)Nc1ccn([C@@H]2O[C@H](CO)[C@@H](O)C2(F)F)c(=O)n1)COCCC(=O)NCC(=O)NCCCC(=O)NCC(=O)Nc1ccn([C@@H]2O[C@H](CO)[C@@H](O)C2(F)F)c(=O)n1]

To a stirring solution of 425 (84 mg, 0.05 mmol) in CH₂C₁₂ (3 mL) at room temperature was added TFA (3 mL), the reaction mixture was stirred for 0.5 h at room temperature, concentrated to afford 426 (80 mg, 100% yield) as a colorless liquid. ESI MS m/z: [M+H]⁺ calcd for C₆₂H₈₇F₆N₁₇O₂₅:1585.46; found 1585.46.

### Example 299. Synthesis of compound 427

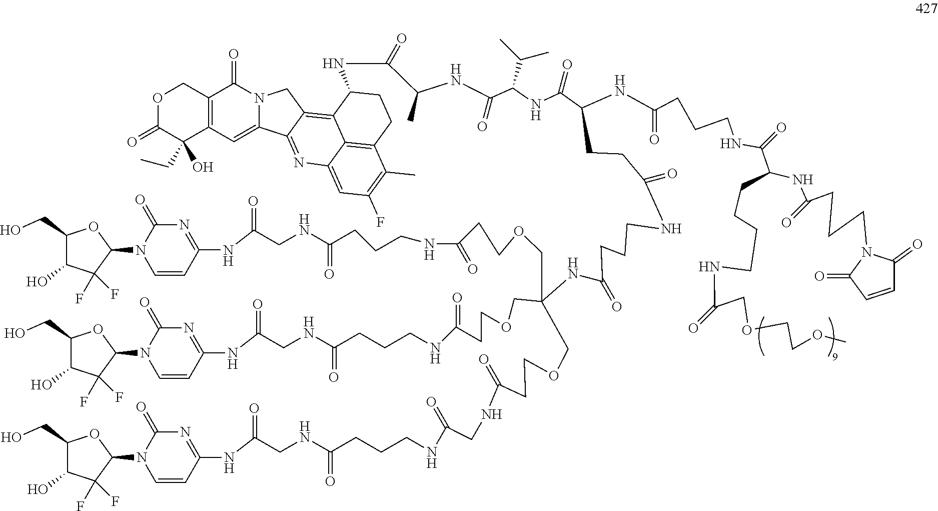

To a stirring solution of 426 (80 mg, 0.05 mmol) in DMF (2 mL) at room temperature were added 210 (80 mg, 0.05 mmol), HATU (23 mg, 0.06 mmol) and DIEA (10 mg, 0.08 mmol). The reaction mixture was stirred for 0.5 h at room temperature, purified by pre-HPLC, lyophilized to afford 427 (58 mg, 36% yield) as a white solid. ESI MS m/z: [2M+H]⁺calcd for C₁₃₈H₁₉₄F₇N₂₇O₄₉:1575.10; found 1575.01.

### Example 300. Synthesis of compound 3, 3′-((4-((tert-butoxycarbonyl)amino)-butanoyl)azanediyl)dipropionic acid (430)

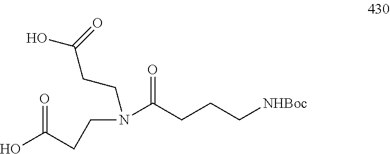 [SMILES: CC(C)(C)OC(=O)NCCCC(=O)N(CCC(=O)O)CCC(=O)O]

To a stirring solution of 429 (500 mg, 3.103 mmol) in DMF (2 mL) at 40° C. were added DIEA (1.6 g, 12.410 mmol) and 198 (1.40 g, 4.654 mmol) in sequence. The resulting mixture was stirred at room temperature for 16 h before HPLC reverse-phase purification directly. Lyophilization afforded 430 (120 mg, 14%).HRMS (ESI): calcd for C₁₅H₂₆N₂O₇ [M+H]⁺ 346.38, found 346.38.

### Example 301. Synthesis of tert-butyl (4-(bis (3-((2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)amino)-3-oxopropyl)amino)-4-oxobutyl)carbamate (431)

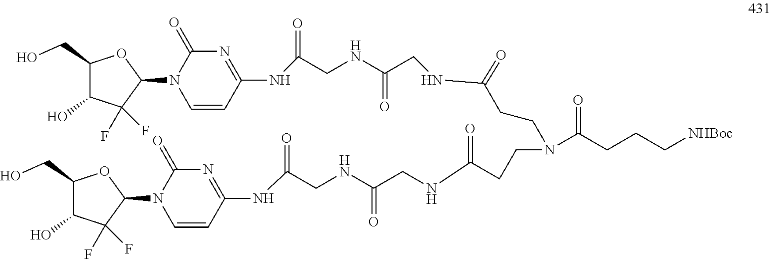 [SMILES: CC(C)(C)OC(=O)NCCCC(=O)N(CCC(=O)NCC(=O)NCC(=O)Nc1ccn([C@@H]2O[C@H](CO)[C@@H](O)C2(F)F)c(=O)n1)CCC(=O)NCC(=O)NCC(=O)Nc1ccn([C@@H]2O[C@H](CO)[C@@H](O)C2(F)F)c(=O)n1]

To a stirring solution of 430 (120.0 mg, 0.346 mmol) in DMF (5 mL) at room temperature were added 410 (360 mg, 0.954 mmol), HATU (395.19 mg, 1.039 mmol) and DIEA (179.11 mg, 1.386 mmol). The resulting mixture was stirred at room temperature for 15 min before it was concentrated in vacuo. HPLC reverse-phase purification and lyophilization afforded 431 (252.0 mg, 64%).

### Example 302. Synthesis of 4-amino-N, N-bis (3-((2-((2-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-2-oxoethyl)amino)-2-oxoethyl)amino -3-oxopropyl)butanamide (432)

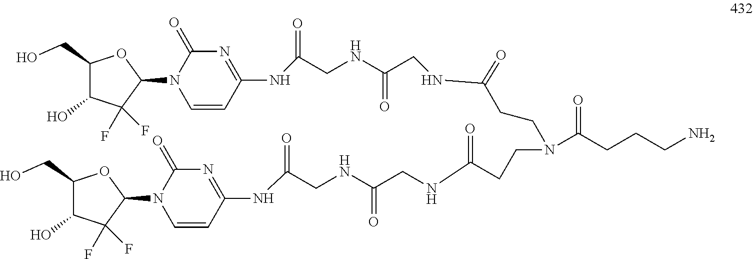 [SMILES: NCCCC(=O)N(CCC(=O)NCC(=O)NCC(=O)Nc1ccn([C@@H]2O[C@H](CO)[C@@H](O)C2(F)F)c(=O)n1)CCC(=O)NCC(=O)NCC(=O)Nc1ccn([C@@H]2O[C@H](CO)[C@@H](O)C2(F)F)c(=O)n1]

A stirring solution of 431 (100.0 mg, 0.094 mmol) in DCM (5 mL) and TFA (1 mL) at room temperature. The resulting mixture was stirred at room temperature for further 1H before it was concentrated in vacuo. The product was triturated from MTBE to afforded 432 (90.6 mg, 1000%).

### Example 303. Synthesis of compound (433)

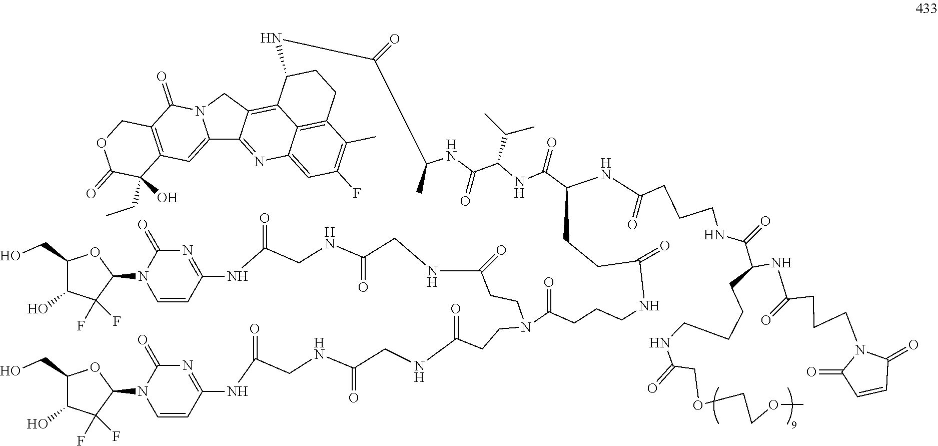

To a stirring solution of 432 (48 mg, 0.05 mmol) in DMF (2 mL) at room temperature were added 210 (80 mg, 0.05 mmol), HAT U (23 mg, 0.06 mmol) and DIEA (10 mg, 0.08 mmol). The reaction mixture was stirred for 0.5 h at room temperature, purified by pre-HPLC, lyophilized to afford 433 (31 mg, 26% yield) as a white solid.

### Example 304. Synthesis of tert-butyl ((S)-1-(((S)-1-((1-((2R, 4R, 5R)-4-((tert-butoxycarbonyl)oxy)-5-(((tert-butoxycarbonyl)oxy)methyl)-3, 3-difluorotetrahydrofuran-2-yl)-2-oxo-1, 2-dihydropyrimidin-4-yl)amino)-1-oxopropan-2-yl)amino)-3-methyl-1-oxobutan-2-yl)carbamate (435)

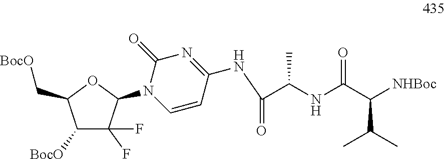

To a stirring solution of 192 (1.0 g, 2.158 mmol) in MeCN (20 mL) at 0° C. was added 129 (0.75 g, 2.589 mmol), 1-Methylimidazole (0.53 g, 6.473 mmol) and TCFH (0.91 g, 3.237 mmol) in sequence. The resulting mixture was stirred 0° C. for 12 h before it was quenched with NaCl. The layers were separated, and the aqueous layer was extracted with CH₂Cl₂. The combined organic layers were dried (Na₂SO₄) and concentrated in vacuo. Flash column chromatography (silica gel, Mehock=5%-8%) afforded 435 (1.7 g, 100%). HRMS (ESI): calcd for C₃₂H₄₉F₂N₅O₁₂ [M+H]⁺ 733.76, found 733.76.

### Example 305. Synthesis of (S)-2-amino-N—((S)-1-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-1-oxopropan-2-yl)-3-methylbutanamide (436)

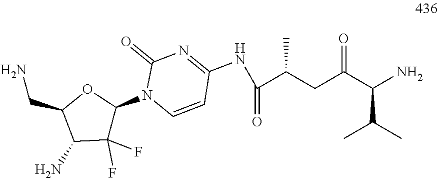 [SMILES: CC(C)[C@H](N)C(=O)C[C@@H](C)C(=O)Nc1ccn([C@@H]2O[C@H](CN)[C@@H](N)C2(F)F)c(=O)n1]

A stirring solution of 435 (0.7 g, 0.954 mmol) in DCM (10 mL) and TFA (3 mL) at room temperature. The resulting mixture was stirred at room temperature for further 1H before it was concentrated in vacuo. The product was triturated from DCM: EA=1:1 to afforded 436 (425.2 mg, 100%). HRMS (ESI): calcd for C₁₇H₂₆F₂N₅O₆[M+H]⁺ 433.41, found 433.41.

### Example 306. Synthesis of tert-butyl (4-(bis(3-(((S)-1-(((S)-1-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-1-oxopropan-2-yl)amino)-3-methyl-1-oxobutan-2-yl)amino)-3-oxopropyl)amino)-4-oxobutyl)carbamate (437)

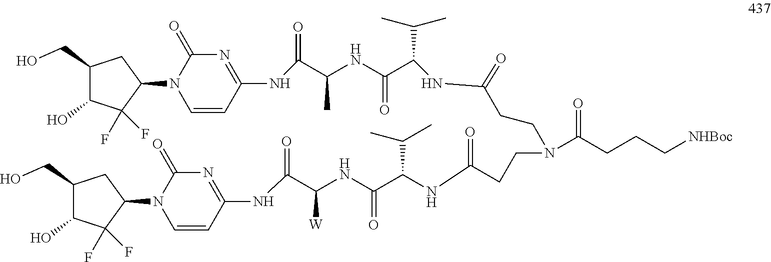

To a stirring solution of 430 (120.0 mg, 0.346 mmol) in DMF (5 mL) at room temperature were added 436 (413.47 mg, 0.954 mmol), HATU (395.19 mg, 1.039 mmol) and DIEA (179.11 mg, 1.386 mmol). The resulting mixture was stirred at room temperature for 15 min before it was concentrated in vacuo. HPLC reverse-phase purification and lyophilization afforded 437 (262.0 mg, 64%). HRMS (ESI): calcd for C₄₉H₇₃F₄N₁₂O₁₇ [M+H]⁺ 1177.18, found 1177.18.

### Example 307. Synthesis of 4-amino-N, N-bis(3-(((S)-1-(((S)-1-((1-((2R, 4R, 5R)-3, 3-difluoro-4-hydroxy-5-(hydroxymethyl)tetrahydrofuran-2-yl)-2-oxo-1, 2-dihydropyrimidin-4-yl)amino)-1-oxopropan-2-yl)amino)-3-methyl-1-oxobutan-2-yl)amino)-3-oxopropyl)butanamide (438)

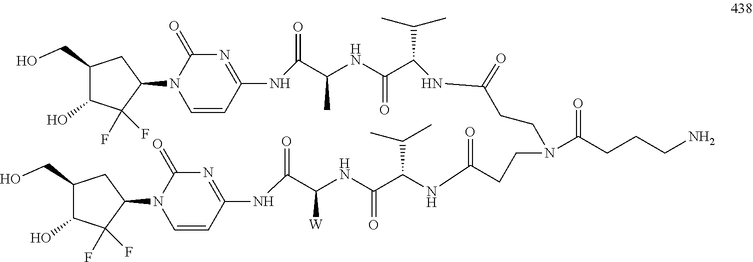

A stirring solution of 437 (100.0 mg, 0.085 mmol) in DCM (5 mL) was added TFA (1 mL) at room temperature. The resulting mixture was stirred at room temperature for 1 h before it was concentrated in vacuo. The product was triturated from MTBE to afforded 438 (90.0 mg, 100%). HRMS (ESI): calcd for C₄₄H₆₅F₄N₁₂O₁₅ [M+H]⁺ 1077.06, found 1077.06.

### Example 308. Synthesis of compound (439)

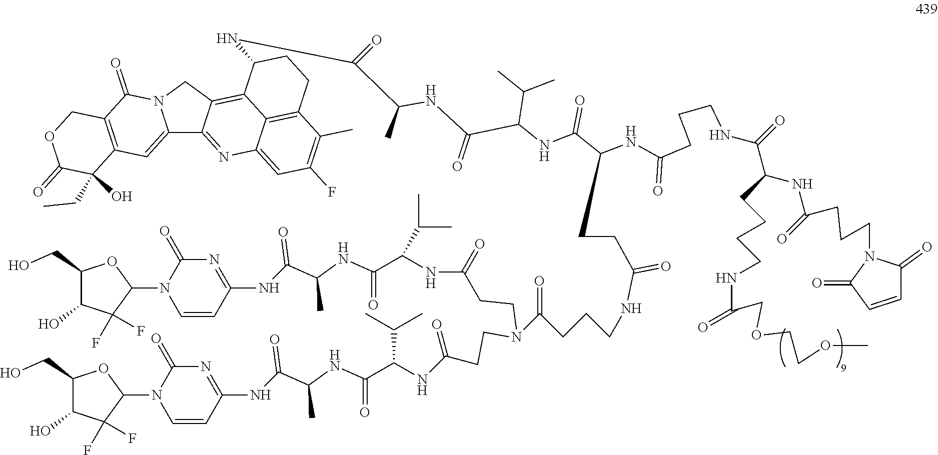

To a stirring solution of 438 (50.94 mg, 0.047 mmol) in DMF (2 mL) at room temperature were added 210 (50.0 mg, 0.032 mmol), HATU (17.98 mg, 0.047 mmol) and DIEA (12.23 mg, 0.095 mmol). The resulting mixture was stirred at room temperature for 15 min before it was concentrated in vacuo. HPLC (MeCN: H2O (0.1% TFA)=38%) reverse-phase purification and lyophilization afforded 439 (21.1 mg, 25%). HRMS (ESI): calcd for C₁₂₀H₁₇₆F5N₂₂O₃₉ [M+H]⁺ 2644.83, found 2644.83.

### Example 309. Synthesis of (S)-(2-((((9H-fluoren-9-yl)methoxy)carbonyl)amino)propanamido)methyl acetate (442)

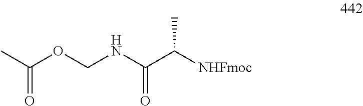

To a stirring solution of 441 (10.1 g, 27.41 mmol) in toluene (30 mL) were added lead tetraacetate (15.0 g, 33.83 mmol), pyridine (2.60 g, 32.90 mmol), at room temperature. The reaction mixture was stirred for 1 h at 70° C. under nitrogen, filtered, concentrated and purified by silica gel column, eluted with petroleum ether and ethyl acetate, concentrated to afford 442 (5.8 g, 55%) as a white solid.

### Example 310. Synthesis of benzyl (S)-4-(((benzyloxy)carbonyl)amino)-5-(((S)-1-(tert-butoxy)-3-methyl-1-oxobutan-2-yl)amino)-5-oxopentanoate (445)

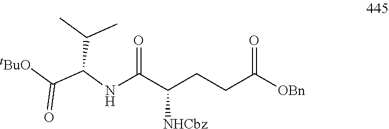 [SMILES: CC(C)[C@H](NC(=O)[C@H](CCC(=O)OCc1ccccc1)NC(=O)OCc1ccccc1)C(=O)OC(C)(C)C]

To a stirring solution of 443 (1.73 g, 10 mmol) in DCM (150 mL) at room temperature were added 444 (3.71 g, 10 mmol), HATU (4.18 g, 11 mmol) and DIPEA (2.58 g, 20 mmol). The reaction mixture was stirred for 2 h at room temperature, washed with water (200 mL), brine (200 mL), dried over anhydrous Na₂SO₄, filtered and concentrated in vacuo, purified by silica gel column, eluted with CH2Cl2 and methanol, concentrated to give 4.73g of white solid (445, 90% yield).

### Example 311. Synthesis of ((S)-5-(benzyloxy)-2-(((benzyloxy)carbonyl)amino)-5-oxopentanoyl)-L-valine (446)

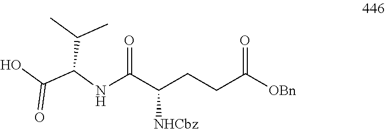 [SMILES: CC(C)[C@H](NC(=O)[C@H](CCC(=O)OCc1ccccc1)NC(=O)OCc1ccccc1)C(=O)O]

To a stirring solution of 445 (2.63 g, 5 mmol) in CH₂Cl₂ (30 mL) at room temperature was added TFA (30 mL), the reaction mixture was stirred for 0.5 h at room temperature, concentrated to afford 446 (2.35 g, 100% yield) as a white solid.

### Example 311. Synthesis of (9H-fluoren-9-yl)methyl ((S)-1-(((3-(((S)-4, 11-diethyl-8-fluoro-4-hydroxy-3, 14-dioxo-3, 4, 12, 14-tetrahydro-1H-pyrano[3′, 4′:6, 7]indolizino[l, 2-b]quinolin-9-yl)oxy)propoxy)methyl)amino)-1-oxopropan-2-yl)carbamate (448)

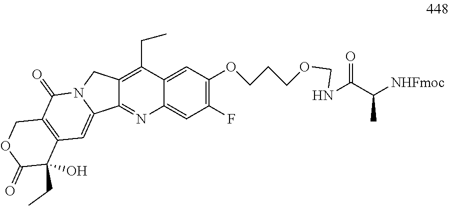

To a stirring solution of 447 (2.0 g, 4.27 mmol) in THF (30 mL) were added 442 (2.1 g, 5.49 mmol), p-toluenesulfonic acid (0.37 g, 24.36 mmol) at room temperature. The reaction mixture was stirred overnight at room temperature, concentrated and purified by a silica gel column, eluted with CH2Cl2 and methanol, concentrated to afford 448 (960 mg, 28%) as light yellow solid.

### Example 312. Synthesis of (S)-2-amino-N-((3-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)propoxy)methyl)propanamide (449)

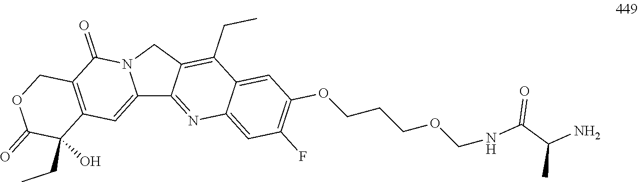 [SMILES: CCc1c2c(nc3cc(F)c(OCCCOCNC(=O)[C@H](C)N)cc13)-c1cc3c(c(=O)n1C2)COC(=O)[C@]3(O)CC]

To a stirring solution of 448 (960 mg, 1.21 mmol) in DMF (10 mL) at room temperature was added piperidine (1 mL), the reaction mixture was stirred for 0.5 h at room temperature, concentrated to afford the crude, crude was slurring by petroleum ether, filtered, dried to afford 449 (630 mg, 910%) as light yellow solid.

### Example 313. Synthesis of benzyl (8S, 11S, 14S)-14-(((benzyloxy)carbonyl)amino)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11-isopropyl-8-methyl-7, 10, 13-trioxo-4-oxa-6, 9, 12-triazaheptadecan-17-oate (450)

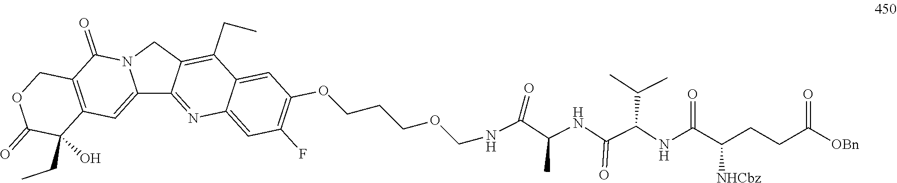 [SMILES: CCc1c2c(nc3cc(F)c(OCCCOCNC(=O)[C@H](C)NC(=O)[C@@H](NC(=O)[C@H](CCC(=O)OCc4ccccc4)NC(=O)OCc4ccccc4)C(C)C)cc13)-c1cc3c(c(=O)n1C2)COC(=O)[C@]3(O)CC]

To a stirring solution of 449 (568 mg, 1.00 mmol) in DMF (20 mL) were added 446 (471 mg, 1.00 mmol), HATU (418 mg, 1.10 mmol) and DIPEA (258 mg, 2.00 mmol) at room temperature. The reaction mixture was stirred for 2 h at room temperature, DCM (200 mL) was added, washed with water (200 mL), brine (200 mL), dried over anhydrous Na₂SO₄, filtered and concentrated in vacuo, purified by silica gel column, eluted with CH2Cl2 and methanol, concentrated to give 763 mg of white solid (450, 75% yield).

### Example 314. Synthesis of (8S, 11S, 14S)-14-amino-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-11-isopropyl-8-methyl-7, 10, 13-trioxo-4-oxa-6, 9, 12-triazaheptadecan-17-oic acid (451)

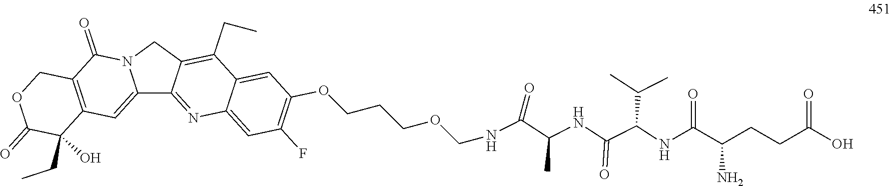 [SMILES: CCc1c2c(nc3cc(F)c(OCCCOCNC(=O)[C@H](C)NC(=O)[C@@H](NC(=O)[C@@H](N)CCC(=O)O)C(C)C)cc13)-c1cc3c(c(=O)n1C2)COC(=O)[C@]3(O)CC]

To a stirring solution of 450 (763 mg, 0.75 mmol) in MeOH (50 mL) at room temperature was added, 10% Pd/C (200 mg), the mixture was stirred for 5 h under hydrogen, filtered, and concentrated to give 598 mg of white solid (451, 100% yield).

### Example 315. Synthesis of (8S, 11S, 14S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-14-(4-(2, 5-dioxo-2, 5-dihydro-1H-pyrrol-1-yl)butanamido)-11-isopropyl-8-methyl-7, 10, 13-trioxo-4-oxa-6, 9, 12-triazaheptadecan-17-oic acid (452)

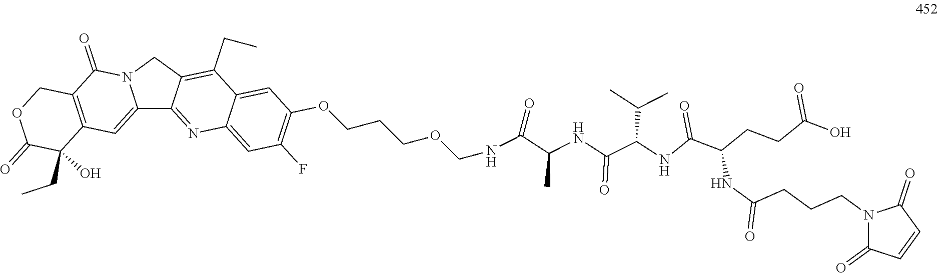 [SMILES: CCc1c2c(nc3cc(F)c(OCCCOCNC(=O)[C@H](C)NC(=O)[C@@H](NC(=O)[C@H](CCC(=O)O)NC(=O)CCCN4C(=O)C=CC4=O)C(C)C)cc13)-c1cc3c(c(=O)n1C2)COC(=O)[C@]3(O)CC]

To a mixture of 451 (598 g, 0.75 mmol) and compound 19 (266 mg, 1.00 mmol) in DMF (20 mL) was added DIEA (259 mg, 2.00 mmol) at r.t. The mixture was stirred at r.t. for 1 h and purified by silica gel column, eluted with DCM and methanol, concentrated to give 577 mg of white solid (compound 452, 80% yield).

### Example 316. Synthesis of 2, 5-dioxopyrrolidin-1-yl (8S, 11S, 14S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-14-(4-(2, 5-dioxo-2, 5-dihydro-1H-pyrrol-1-yl)butanamido)-11-isopropyl-8-methyl-7, 10, 13-trioxo-4-oxa-6, 9, 12-triazaheptadecan-17-oate (453)

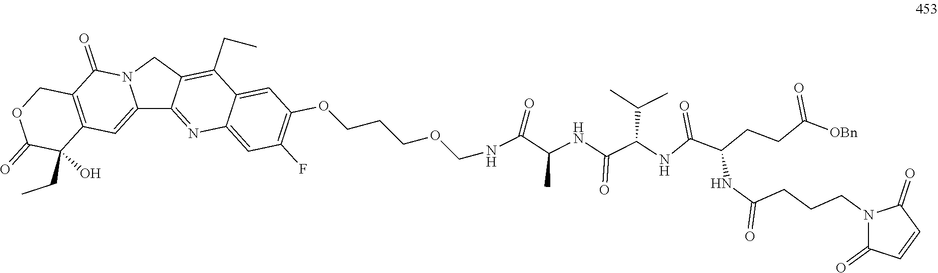 [SMILES: CCc1c2c(nc3cc(F)c(OCCCOCNC(=O)[C@H](C)NC(=O)[C@@H](NC(=O)[C@H](CCC(=O)OCc4ccccc4)NC(=O)CCCN4C(=O)C=CC4=O)C(C)C)cc13)-c1cc3c(c(=O)n1C2)COC(=O)[C@]3(O)CC]

To a stirring solution of 452 (308 mg, 0.32 mmol) in CH₂Cl₂ (25 mL) were added NHS (50 mg, 0.48 mmol) and EDCI (250 mg, 1.29 mmol) at room temperature. The reaction mixture was stirred at room temperature for 4h, washed with brine (10 mL*2), dried over anhydrous Na₂SO₄ and concentrated in vacuo to afford 453 (339 mg, 100% yield).

### Example 317. Synthesis of 2-((2S, 5R, 8S, 11S)-8-(4-aminobutyl)-5-benzyl-11-(3-guanidinopropyl)-3, 6, 9, 12, 15-pentaoxo-1, 4, 7, 10, 13-pentaazacyclopentadecan-2-yl)acetic acid (454)

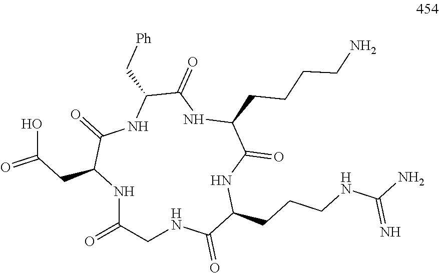 [SMILES: N=C(N)NCCC[C@@H]1NC(=O)[C@H](CCCCN)NC(=O)[C@@H](Cc2ccccc2)NC(=O)[C@H](CC(=O)O)NC(=O)CNC1=O]

To a stirring solution of 291 (330 mg, 0.50 mmol) in CH2Cl2 (10 mL) was added TFA (10 mL) at room temperature. The mixture was stirred for 2h, concentrated and purified by pre-HPLC, lyophilized to afford 454 (271 mg, 90%) as a white solid.

### Example 318. Synthesis of 2-((2S, 5R, 8S, 11S)-5-benzyl-8-((8S, 11S, 14S)-1-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-14-(4-(2, 5-dioxo-2, 5-dihydro-1H-pyrrol-1-yl)butanamido)-11-isopropyl-8-methyl-7, 10, 13, 17-tetraoxo-4-oxa-6, 9, 12, 18-tetraazadocosan-22-yl)-11-(3-guanidinopropyl)-3, 6, 9, 12, 15-pentaoxo-1, 4, 7, 10, 13-pentaazacyclopentadecan-2-yl)acetic acid (455)

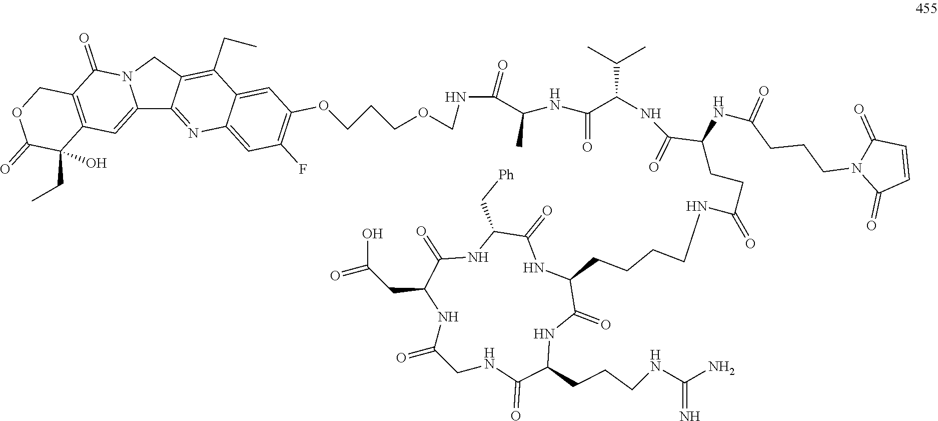 [SMILES: CCc1c2c(nc3cc(F)c(OCCCOCNC(=O)[C@H](C)NC(=O)[C@@H](NC(=O)[C@H](CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CCCN4C(=O)C=CC4=O)C(C)C)cc13)-c1cc3c(c(=O)n1C2)COC(=O)[C@]3(O)CC]

To a stirring solution of 453 (106 mg, 0.10 mmol) in DMF (5 mL) were added 454 (61 mg, 0.10 mmol) and DIPEA (26 mg 0.20 mmol) at room temperature. The mixture was stirred for 2h, concentrated and purified by pre-HPLC, lyophilized to afford 455 (77 mg, 50%) as a white solid.

### Example 319. Synthesis of tert-butyl N2-((benzyloxy)carbonyl)-N6-(tert-butoxycarbonyl)-L-lysyl-L-valinate (457)

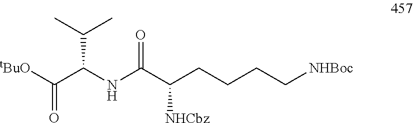 [SMILES: CC(C)[C@H](NC(=O)[C@H](CCCCNC(=O)OC(C)(C)C)NC(=O)OCc1ccccc1)C(=O)OC(C)(C)C]

To a solution of tert-butyl L-valinate (2.28 g, 13.14 mmol, 1.0 eq) and 1 (5.0 g, 14.46 mmol, 1.1 eq) in DMF (20 mL) was added HATU (6 g, 15.771 mmol, 1.2 eq), then added DIPEA (5.1 g, 39.43 mmol, 2.0 eq). The mixture was stirred at RT for 1 h. The reaction mixture was extracted with DCM (50 mL*3) and H₂O (50 mL). The combined organic layers were washed with H₂O (50 mL*3), dried over Na₂SO₄and concentrated in vacuo. The residue was purified by column chromatography on silica gel (eluted with DCM/MeOH=50/1) to afford 457 (7.04 g, 100% yield) as a yellow oil.

### Example 320. Synthesis of ((benzyloxy)carbonyl)-L-lysyl-L-valine (458) HO NH₂

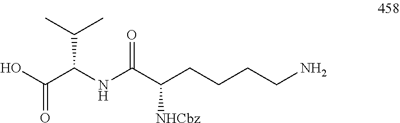 [SMILES: CC(C)[C@H](NC(=O)[C@H](CCCCN)NC(=O)OCc1ccccc1)C(=O)O]

To a solution of 457 (7.04 g, 13.14 mmol) in DCM (40 mL) was added TFA (40 mL). The mixture was stirred at RT for 1 h. The reaction mixture was concentrated in vacuo to afford 458 (4.99 g, 100%) as a colorless oil.

### Example 321. Synthesis of ((S)-17-(((benzyloxy)carbonyl)amino)-1-(4-(tert-butoxycarbonyl)phenyl)-1, 11-dioxo-5, 8-dioxa-2, 12-diazaoctadecan-18-oyl)-L-valine (459)

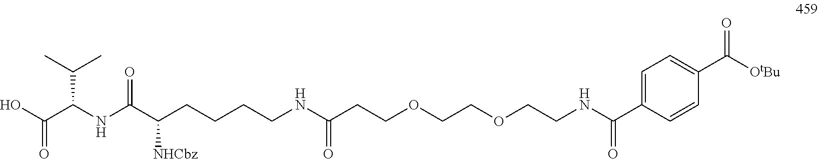 [SMILES: CC(C)[C@H](NC(=O)[C@H](CCCCNC(=O)CCOCCOCCNC(=O)c1ccc(C(=O)OC(C)(C)C)cc1)NC(=O)OCc1ccccc1)C(=O)O]

To a stirring solution of 7 (211 mg, 0.554 mmol) in CH₂Cl₂ (10 mL) at room temperature was added NHS (96 mg, 0.831 mmol) and EDCI (212 mg, 1.108 mmol,). The reaction mixture was stirred for 3 h at room temperature. The reaction mixture was washed brine (15 mL*2), dried over anhydrous Na₂SO₄and concentrated in vacuo to afford crude. To the crude in DCM (10 mL) were added 458 (210 mg, 0.554 mmol) and DIEA (0.193 mL, 1.108 mmol). The mixture was stirred at room temperature overnight. The reaction mixture was concentrated in vacuo. The residue was purified by prep-HPLC to afford 459 (333 mg, 81% yield).

### Example 322. Synthesis of allyl (17S, 20S)-17-(((benzyloxy)carbonyl)amino)-1-(4-(tert-butoxycarbonyl)phenyl)-20-isopropyl-1, 11, 18-trioxo-5, 8-dioxa-2, 12, 19-triazahenicosan-21-oate (460)

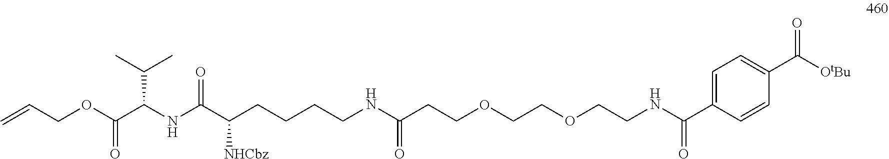 [SMILES: C=CCOC(=O)[C@@H](NC(=O)[C@H](CCCCNC(=O)CCOCCOCCNC(=O)c1ccc(C(=O)OC(C)(C)C)cc1)NC(=O)OCc1ccccc1)C(C)C]

To a solution of 459 (8.5 g, 11.75 mmol, 1.0 eq) and allyl bromide (4.27 g, 35.25 mmol, 3.0 eq) in CH₃CN (50 mL) was added K₂CO₃ (3.25 g, 23.50 mmol, 2.0 eq). The mixture was stirred at 50° C. overnight. The reaction mixture was filtered with celite. The filtrate was concentrated in vacuo. The residue was purified by column chromatography on silica gel (eluted with DCM/MeOH=50/1~30/1) to afford 460 (6.16 g, 69% yield) as a yellow oil.

### Example 323. Synthesis of 4-(((15S, 18S)-15-(((benzyloxy)carbonyl)amino)-18-isopropyl-9, 16, 19-trioxo-3, 6, 20-trioxa-10, 17-diazatricos-22-en-1-yl)carbamoyl)benzoic acid (461)

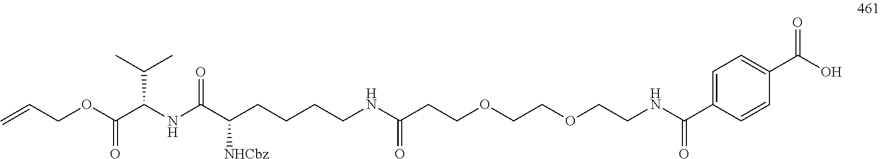 [SMILES: C=CCOC(=O)[C@@H](NC(=O)[C@H](CCCCNC(=O)CCOCCOCCNC(=O)c1ccc(C(=O)O)cc1)NC(=O)OCc1ccccc1)C(C)C]

To a solution of 9 (6.16 g, 8.16 mmol, 1.0 eq) in DCM (50 mL) was added TFA (50 mL). The mixture was stirred at RT for 1 h. The reaction mixture was concentrated in vacuo to afford 10 (5.7 g, crude) as a yellow oil.

### Example 324. Synthesis of allyl ((S)-1-(4-((4-(benzyloxy)-4-oxobutyl)carbamoyl)phenyl)-17-(((benzyloxy)carbonyl)amino)-1, 11-dioxo-5, 8-dioxa-2, 12-diazaoctadecan-18-oyl)-L-valinate (462)

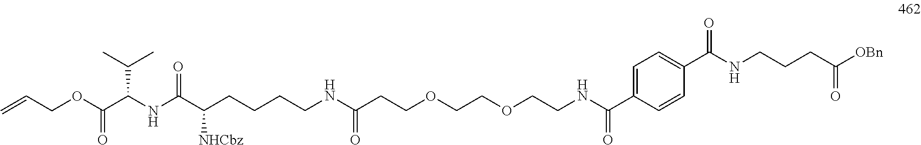 [SMILES: C=CCOC(=O)[C@@H](NC(=O)[C@H](CCCCNC(=O)CCOCCOCCNC(=O)c1ccc(C(=O)NCCCC(=O)OCc2ccccc2)cc1)NC(=O)OCc1ccccc1)C(C)C]

To a solution of 461 (5.7 g, 8.16 mmol, 1.0 eq) and benzyl 4-aminobutanoate (1.73 g, 8.97 mmol, 1.0 eq) in DMF (20 mL) was added HATU (3.72 g, 9.79 mmol, 1.2 eq), then added DIPEA (2.11 g, 16.31 mmol, 2.0 eq). The mixture was stirred at RT for 1 h. The reaction mixture was concentrated in vacuo.

The residue was purified by column chromatography on silica gel (eluted with DCM/MeOH=20/1) to afford 462 (7 g, 98% yield) as a yellow oil.

### Example 325. Synthesis of ((S)-1-(4-((4-(benzyloxy)-4-oxobutyl)carbamoyl)phenyl)-17-(((benzyloxy)carbonyl)amino)-1, 11-dioxo-5, 8-dioxa-2, 12-diazaoctadecan-18-oyl)-L-valine (463)

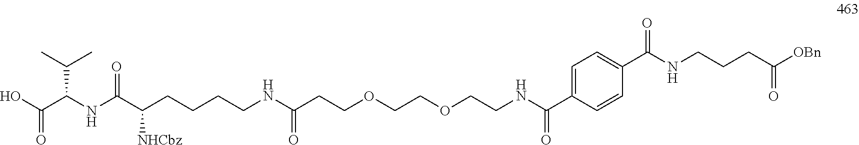 [SMILES: CC(C)[C@H](NC(=O)[C@H](CCCCNC(=O)CCOCCOCCNC(=O)c1ccc(C(=O)NCCCC(=O)OCc2ccccc2)cc1)NC(=O)OCc1ccccc1)C(=O)O]

To a solution of 462 (7 g, 8.009 mmol, 1.0 eq) in DCM (50 mL) was added pyrrolidine (3.62 g, 16.02 mmol, 2.0 eq), then added Pd (PPh₃)₄(190 mg, 0.196 mmol, 0.02 eq). The mixture was stirred at RT for 1 h. The reaction mixture was washed with 1 N aqueous HCl solution (50 mL), H₂O (50 mL), dried over anhydrous Na₂SO₄and concentrated in vacuo. The residue was purified by column chromatography on silica gel (eluted with DCM/MeOH=10/1) to afford 463 (3 g, 36% yield) as a white solid.

### Example 326. Synthesis of benzyl 4-(4-(((15S, 18S, 21S)-15-(((benzyloxy)carbonyl)amino)-28-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-18-isopropyl-21-methyl-9, 16, 19, 22-tetraoxo-3, 6, 25-trioxa-10, 17, 20, 23-tetraazaoctacosyl)carbamoyl)benzamido)butanoate (464)

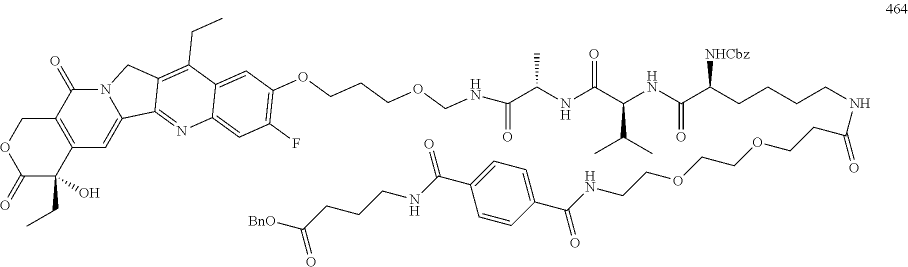 [SMILES: CCc1c2c(nc3cc(F)c(OCCCOCNC(=O)[C@H](C)NC(=O)[C@@H](NC(=O)[C@H](CCCCNC(=O)CCOCCOCCNC(=O)c4ccc(C(=O)NCCCC(=O)OCc5ccccc5)cc4)NC(=O)OCc4ccccc4)C(C)C)cc13)-c1cc3c(c(=O)n1C2)COC(=O)[C@]3(O)CC]

To a stirring solution of 449 (300 mg, 0.52 mmol) in DMF (10 mL) at room temperature were added 463 (455 mg, 0.528 mmol), HATU (301 mg, 0.79 mmol) and DIEA (137 mg, 1.00 mmol), the reaction mixture was stirred for 1 h at room temperature, concentrated and purified by a silica gel column, eluted with CH2Cl2 and methanol, concentrated to afford 464 (745 mg, 100%) as a light yellow solid.

### Example 327. Synthesis of 4-(4-(((15S, 18S, 21S)-15-amino-28-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-18-isopropyl-21-methyl-9, 16, 19, 22-tetraoxo-3, 6, 25-trioxa-10, 17, 20, 23-tetraazaoctacosyl)carbamoyl)benzamido)butanoic acid (465)

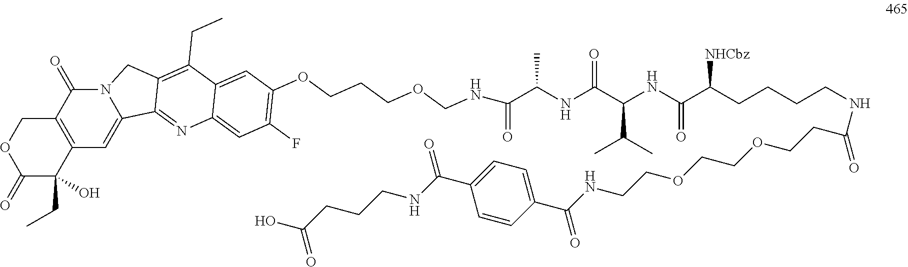 [SMILES: CCc1c2c(nc3cc(F)c(OCCCOCNC(=O)[C@H](C)NC(=O)[C@@H](NC(=O)[C@H](CCCCNC(=O)CCOCCOCCNC(=O)c4ccc(C(=O)NCCCC(=O)O)cc4)NC(=O)OCc4ccccc4)C(C)C)cc13)-c1cc3c(c(=O)n1C2)COC(=O)[C@]3(O)CC]

To a stirring solution of 464 (745 mg, 0.52 mmol) in MeOH (40 mL) at room temperature was added 10% Pd/C (200.0 mg), the reaction mixture was stirred for 5 h under hydrogen at 50° C., filtered, concentrated and purified by pre-HPLC, lyophilized to afford 465 (320 mg, 52%) as a white solid.

### Example 328. Synthesis of 4-(4-(((15S, 18S, 21S)-28-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-15-(4-(2, 5-dioxo-2, 5-dihydro-1H-pyrrol-1-yl)butanamido)-18-isopropyl-21-methyl-9, 16, 19, 22-tetraoxo-3, 6, 25-trioxa-10, 17, 20, 23-tetraazaoctacosyl)carbamoyl)benzamido)butanoic acid (466)

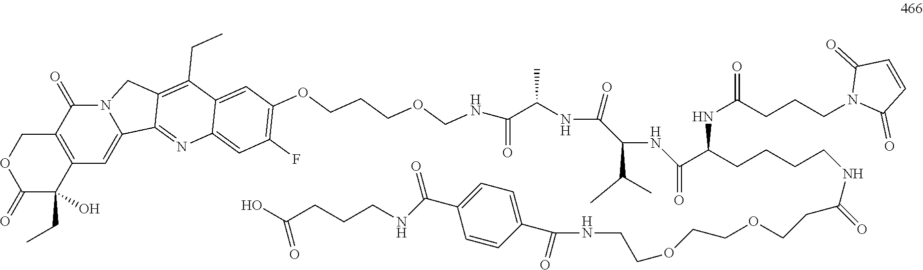 [SMILES: CCc1c2c(nc3cc(F)c(OCCCOCNC(=O)[C@H](C)NC(=O)[C@@H](NC(=O)[C@H](CCCCNC(=O)CCOCCOCCNC(=O)c4ccc(C(=O)NCCCC(=O)O)cc4)NC(=O)CCCN4C(=O)C=CC4=O)C(C)C)cc13)-c1cc3c(c(=O)n1C2)COC(=O)[C@]3(O)CC]

To a stirring solution of 465 (59.0 mg, 0.05 mmol) in DMF (3 mL) at room temperature were added 19 (20.0 mg, 0.07 mmol), DIEA (13.0 mg, 0.10 mmol), the mixture was stirred for 3 h at room temperature, concentrated and purified by pre-HPLC, lyophilized to afford 466 (37.0 mg, 55%) as a white solid.

### Example 329. Synthesis of 2-((2S, 5R, 8S, 11S)-5-benzyl-8-(4-(4-(4-(((15S, 18S, 21S)-28-(((S)-4, 11-diethyl-8-fluoro-4-hydroxy-3, 14-dioxo-3, 4, 12, 14-tetrahydro-1H-pyrano[3′, 4′:6, 7]indolizino[1, 2-b]quinolin-9-yl)oxy)-15-(4-(2, 5-dioxo-2, 5-dihydro-IH-pyrrol-1-yl)butanamido)-18-isopropyl-21-methyl-9, 16, 19, 22-tetraoxo-3, 6, 25-trioxa-10, 17, 20, 23-tetraazaoctacosyl)carbamoyl)benzamido)butanamido)butyl)-11-(3-guanidinopropyl)-3, 6, 9, 12, 15-pentaoxo-1, 4, 7, 10, 13-pentaazacyclopentadecan-2-yl)acetic acid (467)

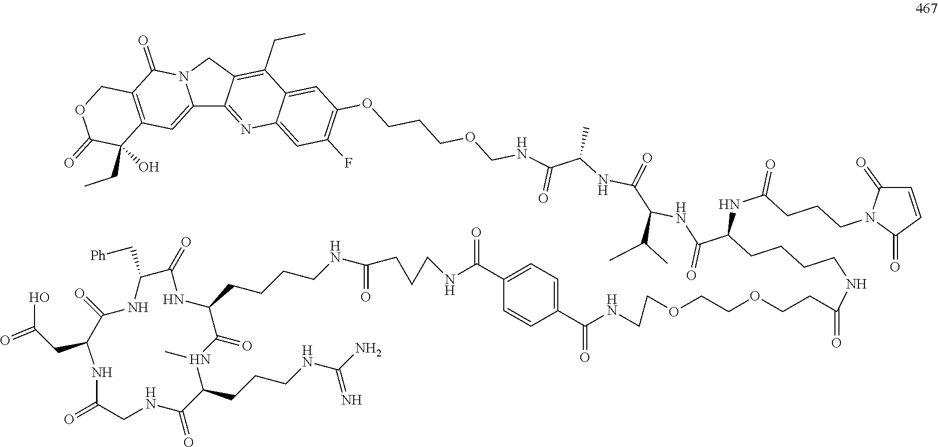 [SMILES: CCc1c2c(nc3cc(F)c(OCCCOCNC(=O)[C@H](C)NC(=O)[C@@H](NC(=O)[C@H](CCCCNC(=O)CCOCCOCCNC(=O)c4ccc(C(=O)NCCCC(=O)NCCCC[C@@H]5NC(=O)[C@@H](Cc6ccccc6)NC(=O)[C@H](CC(=O)O)NC(=O)CNC(=O)[C@H](CCCNC(=N)N)N(C)C5=O)cc4)NC(=O)CCCN4C(=O)C=CC4=O)C(C)C)cc13)-c1cc3c(c(=O)n1C2)COC(=O)[C@]3(O)CC]

To a stirring solution of 466 (37.0 mg, 0.027 mmol) in CH2Cl2 (10 mL) at room temperature were added EDCI (16.0 mg, 0.087 mmol), N-hydroxysuccinimide (10.0 mg, 0.083 mmol), the reaction mixture was stirred for 5 h at room temperature, diluted with CH2Cl2 (30 mL), washed with water (20 mL), dried over anhydrous sodium sulfate, filtered, concentrated to afford crude. To the crude solution in DMF (5 mL) were added 454 (25.0 mg, 0.040 mmol) and DIEA (11.0 mg, 0.080 mmol), the reaction mixture was stirred for 2 h at room temperature, purified by pre-HPLC, lyophilized to afford 467 (21 mg, 40%) as a white solid.

### Example 330. The protocol for solid-phase peptide synthesis (SPPS) including so-called difficult sequences which was based on the widely used Fmoc/tBu strategy, activation of the carboxyl groups by aminium-derived coupling reagents and use of PEG-modified polystyrene resins can be seen from a reference (I. Coin, et al, Nature Protocols, 2007, 0.2 (12), 3247-56 and many references cited thereof). Most peptide sequences listed in this application were customized from Raybow (Hangzhou) and other CROs or CDMOs. Some structures of the synthesized peptide sequences containing a linker of azido terminal are

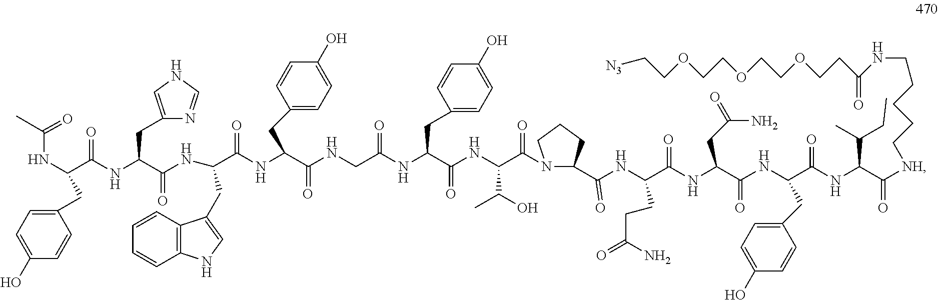 [SMILES: CCC(C)[C@H](NC(=O)[C@H](Cc1ccc(O)cc1)NC(=O)[C@H](CC(N)=O)NC(=O)[C@H](CCC(N)=O)NC(=O)[C@@H]1CCCN1C(=O)[C@@H](NC(=O)[C@H](Cc1ccc(O)cc1)NC(=O)CNC(=O)[C@H](Cc1ccc(O)cc1)NC(=O)[C@H](Cc1c[nH]c2ccccc12)NC(=O)[C@H](Cc1c[nH]cn1)NC(=O)[C@H](Cc1ccc(O)cc1)NC(C)=O)C(C)O)C(=O)NCCCCCNC(=O)CCOCCOCCOCCN=[N+]=[N-]]

10.11 g, 95.6% pure by HPLC, MS (ESI), C₉₅H₁₂₇N₂₂O₂₄ [M+H]⁺ 1959.9394; found, 1961.2355;

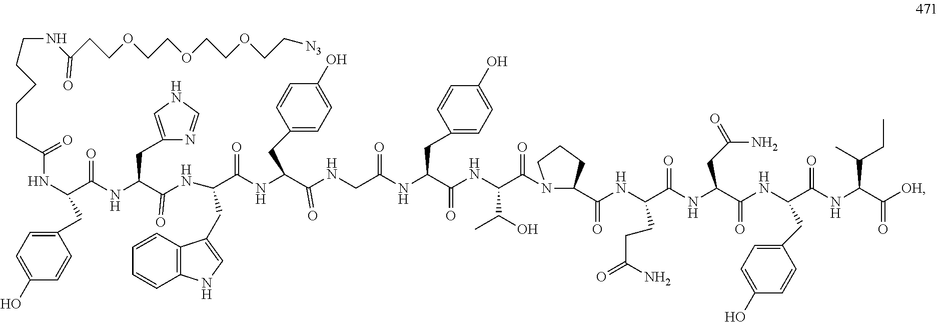 [SMILES: CCC(C)[C@H](NC(=O)[C@H](Cc1ccc(O)cc1)NC(=O)[C@H](CC(N)=O)NC(=O)[C@H](CCC(N)=O)NC(=O)[C@@H]1CCCN1C(=O)[C@@H](NC(=O)[C@H](Cc1ccc(O)cc1)NC(=O)CNC(=O)[C@H](Cc1ccc(O)cc1)NC(=O)[C@H](Cc1c[nH]c2ccccc12)NC(=O)[C@H](Cc1c[nH]cn1)NC(=O)[C@H](Cc1ccc(O)cc1)NC(=O)CCCCCNC(=O)CCOCCOCCOCCN=[N+]=[N-])C(C)O)C(=O)O]

10.51 g, 95.3% pure MS, C₉₄H₁₂₄N₂₁O₂₅ [M+H]⁺ 1946.90781; found, 1948.2260;

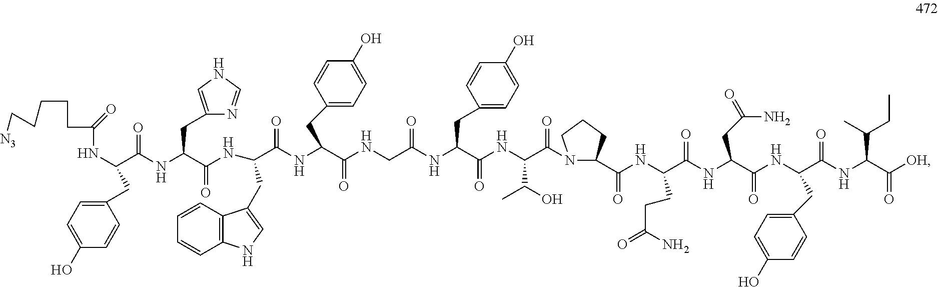 [SMILES: CCC(C)[C@H](NC(=O)[C@H](Cc1ccc(O)cc1)NC(=O)[C@H](CC(N)=O)NC(=O)[C@H](CCC(N)=O)NC(=O)[C@@H]1CCCN1C(=O)[C@@H](NC(=O)[C@H](Cc1ccc(O)cc1)NC(=O)CNC(=O)[C@H](Cc1ccc(O)cc1)NC(=O)[C@H](Cc1c[nH]c2ccccc12)NC(=O)[C@H](Cc1c[nH]cn1)NC(=O)[C@H](Cc1ccc(O)cc1)NC(=O)CCCCCN=[N+]=[N-])C(C)O)C(=O)O]

10.61 g, 96.2% pure, MS, C₈₅H₁₀₇N₂₀O₂₁ [M+H]⁺ 1743.7920; found, 1743.8285;

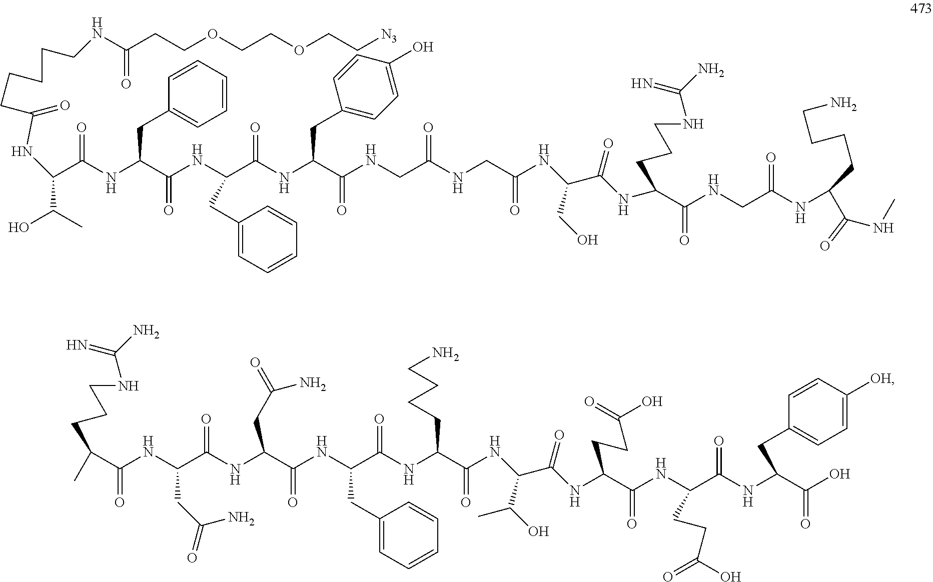

2,05 g, 95.3% pure, MS, C₁₁₇H₁₇₃N₃₄O₃₄ [M+H]⁺ 2599.2694; found, 2599.2978;

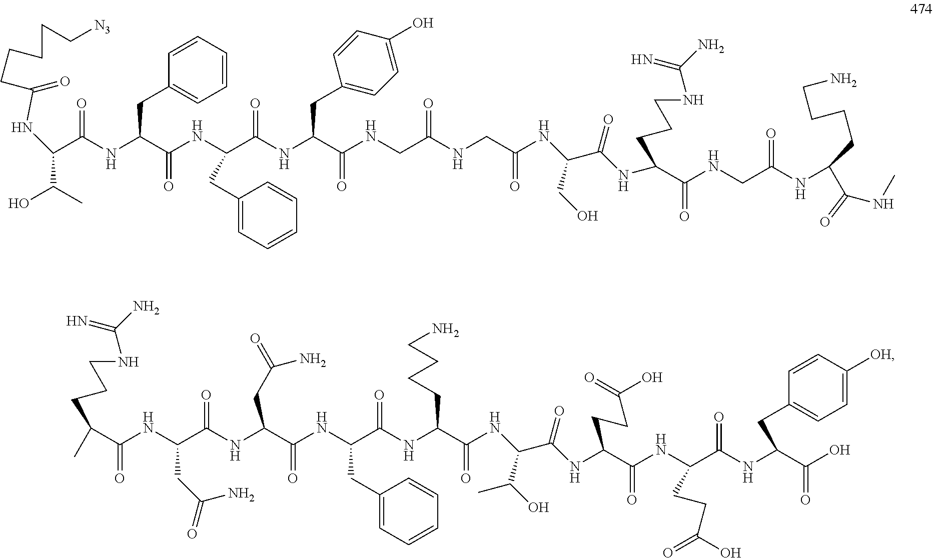

2.13 g, 96.1% pure, MS, C₁₁₀H₁₅₉N₃₂O₃₂ [M+H]⁺ 2440.1799; found, 2440.2045;

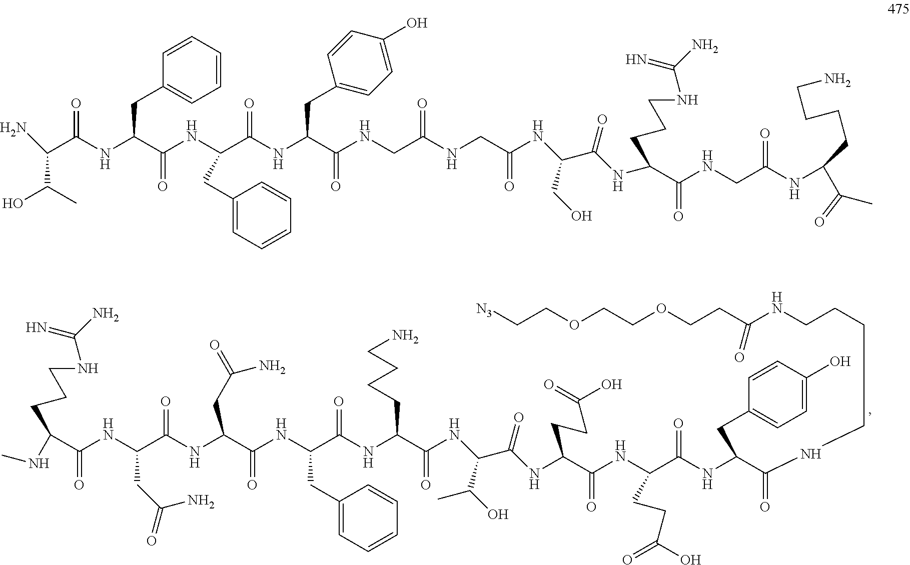

2.32 g, 95.7% pure; MS, C₁₁₅H₁₇₁N₃₄O₃₃ [M+H]⁺ 2556.2749; found, 2556.3038;

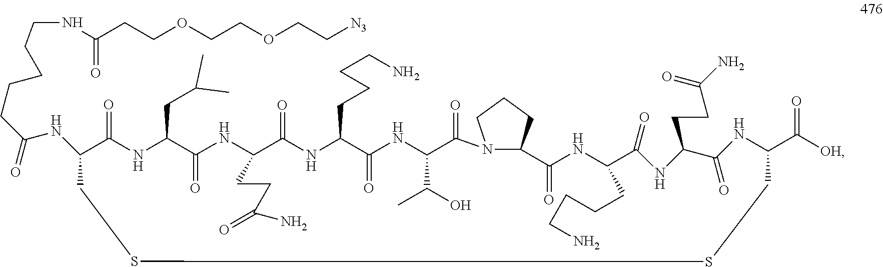 [SMILES: CC(C)C[C@@H]1NC(=O)[C@@H](NC(=O)CCCCCNC(=O)CCOCCOCCN=[N+]=[N-])CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC1=O]

5.51 g, 96.1% pure; MS, C₅₆H₉₈N₁₇O₁₇S₂ [M+H]⁺ 1344.6769; found, 1345.9555;

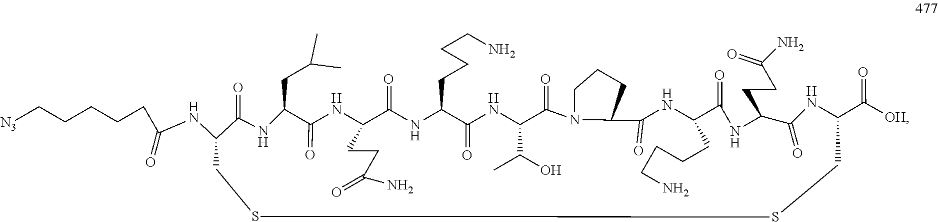

5.19 g, 95.7% pure; MS, C₄₉H₈₅N₁₆O₁₄S₂[M+H]⁺ 1185.5873; found, 1185.6078;

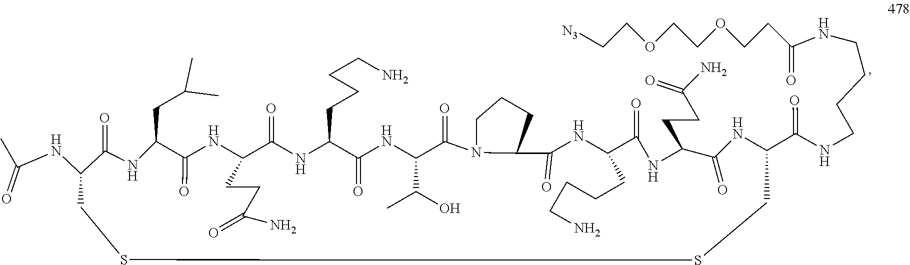

5.42 g, 95.3% pure; MS, C₅₆H₉₉N₁₈O₁₆S₂[M+H]⁺ 1343.6929; found, 1344.90555;

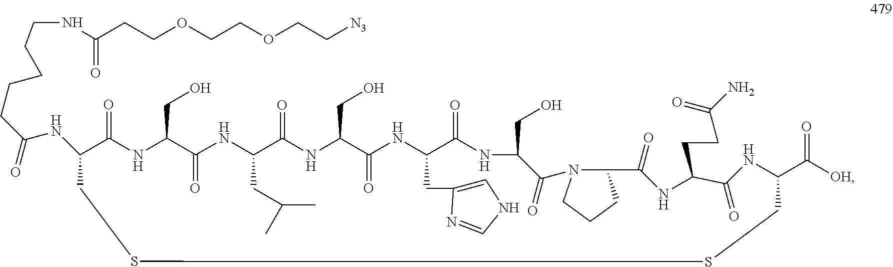 [SMILES: CC(C)C[C@@H]1NC(=O)[C@H](CO)NC(=O)[C@@H](NC(=O)CCCCCNC(=O)CCOCCOCCN=[N+]=[N-])CSSC[C@@H](C(=O)O)NC(=O)[C@H](CCC(N)=O)NC(=O)[C@H]2CCCN2C(=O)[C@H](CO)NC(=O)[C@H](Cc2c[nH]cn2)NC(=O)[C@H](CO)NC1=O]

5.38 g, 95.5% pure; MS, C₅₀H₈₁N₁₆O₁₈S₂[M+H]⁺ 1257.5357; found, 1258.9885;

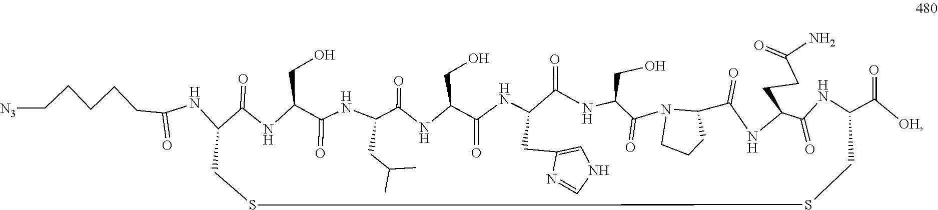

5.29 g, 95.2% pure; MS, C₄₃H₆₈N₁₅O₁₅S₂[M+H]⁺ 1098.4461; found, 1098.4625;

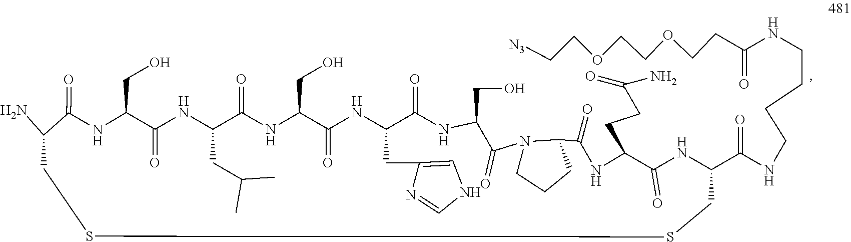

5.42 g, 96.2% pure; MS, C₄₈H₈₀N₁₇O₁₆S₂ [M+H]⁺ 1214.5411; found, 1215.5646;

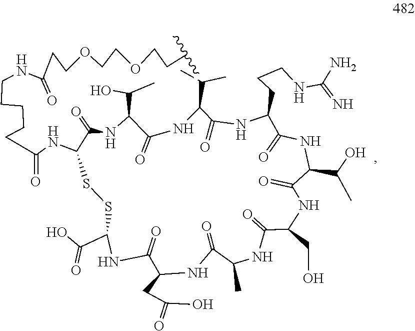

5.13 g, 95.1% pure; MS, C₄₆H₇₉N₁₆O₁₉S₂[M+H]⁺ 1223.5150; found, 1224.7048;

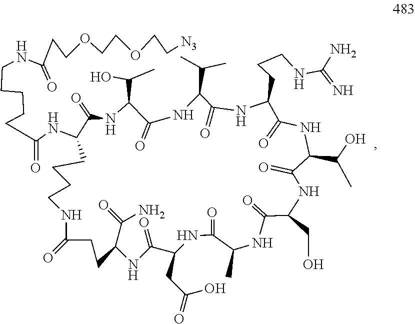

5.33 g, 95.9% pure; MS, C₅₃H₉₃N₁₅O₁₉ [M+H]⁺ 1285.6865; found, 1286.8080;

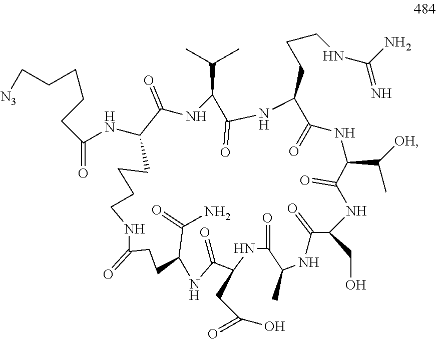 [SMILES: CC(C)[C@@H]1NC(=O)[C@@H](NC(=O)CCCCCN=[N+]=[N-])CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC1=O]

5.47 g, 96.4% pure; MS, C₄₆H₈₀N₁₇O₁₆ [M+H]⁺ 1126.5970; found, 1126.6185;

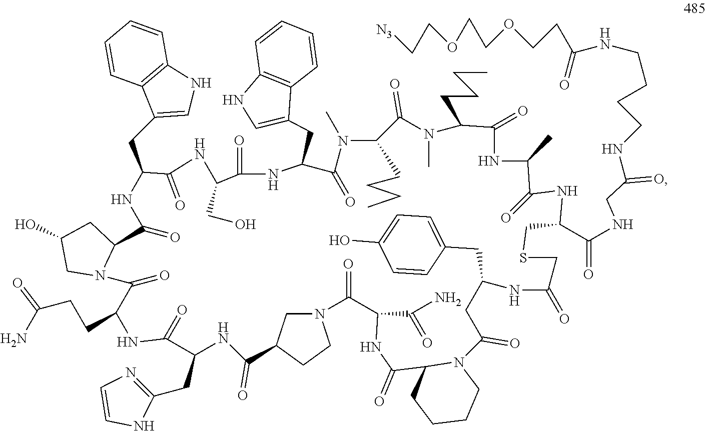 [SMILES: CCCC[C@H]1C(=O)N(C)[C@@H](CCCC)C(=O)N[C@@H](C)C(=O)N[C@H](C(=O)NCC(=O)NCCCCNC(=O)CCOCCOCCN=[N+]=[N-])CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N1C]

5.36 g, 95.2% o pure; MS, C₁₀₀H₁₄₁N₂₆O₂₄S [M+H]⁺ 2122.0334; found, 2123.3883;

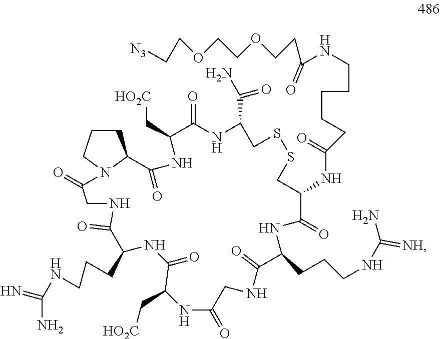 [SMILES: [N-]=[N+]=NCCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

5.11 g, 95.1% pure; MS, C₄₈H₈₁N₂₀O₁₇S₂ [M+H]⁺ 1273.5531; found, 1274.1025;

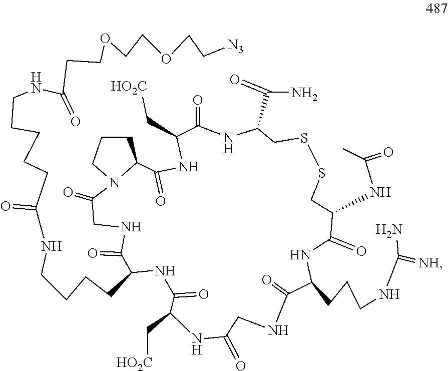 [SMILES: CC(=O)N[C@H]1CSSC[C@@H](C(N)=O)NC(=O)[C@H](CC(=O)O)NC(=O)[C@@H]2CCCN2C(=O)CNC(=O)[C@H](CCCCNC(=O)CCCCCNC(=O)CCOCCOCCN=[N+]=[N-])NC(=O)[C@H](CC(=O)O)NC(=O)CNC(=O)[C@H](CCCNC(=N)N)NC1=O]

MS, 5.16 g, 95.4% pure; C₅₀H₈₃N₁₈O₁₈S₂ [M+H]⁺ 1287.5575; found, 1288.3985;

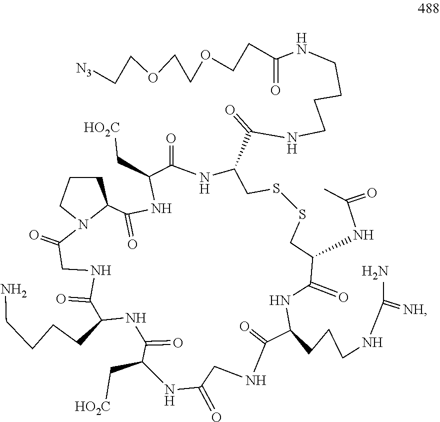 [SMILES: CC(=O)N[C@H]1CSSC[C@@H](C(=O)NCCCCNC(=O)CCOCCOCCN=[N+]=[N-])NC(=O)[C@H](CC(=O)O)NC(=O)[C@@H]2CCCN2C(=O)CNC(=O)[C@H](CCCCN)NC(=O)[C@H](CC(=O)O)NC(=O)CNC(=O)[C@H](CCCNC(=N)N)NC1=O]

5.42 g, 95.7% pure; MS, C₄;H₈₁N₁₈O₁₇S₂[M+H]⁺ 1245.5469; found, 1246.6233;

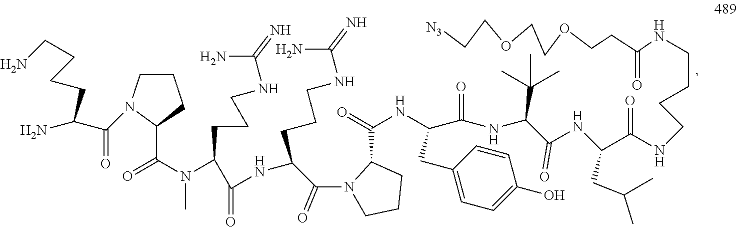 [SMILES: CC(C)C[C@H](NC(=O)[C@@H](NC(=O)[C@H](Cc1ccc(O)cc1)NC(=O)[C@@H]1CCCN1C(=O)[C@H](CCCNC(=N)N)NC(=O)[C@H](CCCNC(=N)N)N(C)C(=O)[C@@H]1CCCN1C(=O)[C@@H](N)CCCCN)C(C)(C)C)C(=O)NCCCCNC(=O)CCOCCOCCN=[N+]=[N-]]

5.89 g, 96.6% pure; MS, C₆₁H₁₀₇N₂₀O₁₂ [M+H]⁺ 1311.8378; found, 1312.9935.

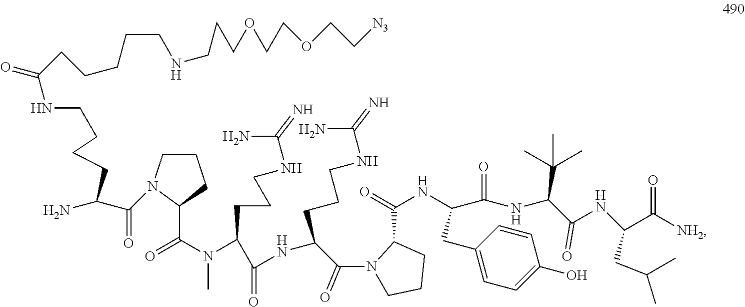 [SMILES: CC(C)C[C@H](NC(=O)[C@@H](NC(=O)[C@H](Cc1ccc(O)cc1)NC(=O)[C@@H]1CCCN1C(=O)[C@H](CCCNC(=N)N)NC(=O)[C@H](CCCNC(=N)N)N(C)C(=O)[C@@H]1CCCN1C(=O)[C@@H](N)CCCCNC(=O)CCCCCNCCCOCCOCCN=[N+]=[N-])C(C)(C)C)C(N)=O]

5.93 g, 96.3% pure; MS, C₆₃Hu ₁N₂₀O₁₂ [M+H]⁺ 1339.8691; found, 1341.1080;

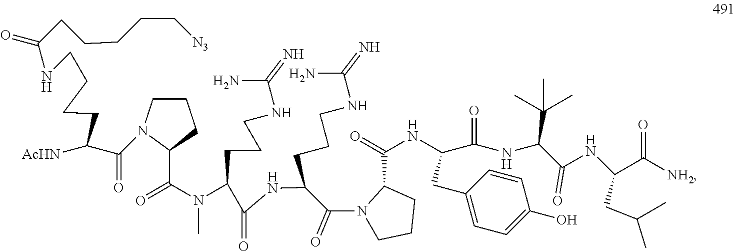 [SMILES: CC(=O)N[C@@H](CCCCNC(=O)CCCCCN=[N+]=[N-])C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(N)=O)C(C)(C)C]

5.77 g, 96.8% pure; MS, C₅₈H₉₈N₁₉O₁₁ [M+H]⁺ 1236.7694; found, 1236.7926;

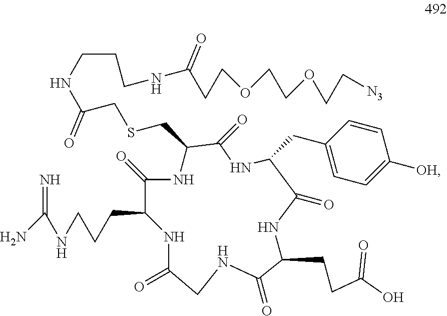 [SMILES: [N-]=[N+]=NCCOCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

5.10 g, 95.1% pure; MS, C₃₇H₅₈N₁₃O₁₂S [M+H]⁺ 908.4049; found, 909.9555;

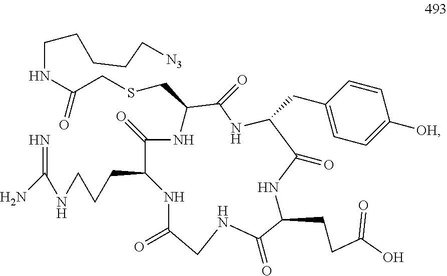 [SMILES: [N-]=[N+]=NCCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

5.04 g, 96.1% pure; MS, C₃₂H₄₉N₁₂O₉S [M+H]⁺ 777.3467; found, 777.3555;

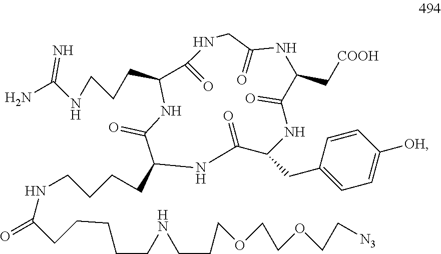 [SMILES: [N-]=[N+]=NCCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

5.09 g, 96.5% pure; MS, C₄₀H₆₅N₁₃O₁₁ [M+H]⁺ 904.5005; found, 905.4166.

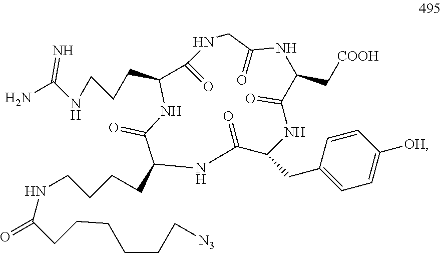 [SMILES: [N-]=[N+]=NCCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

5.23 g, 95.9% pure; MS, C₃₄H₅₃N₁₂O₉[M+H]⁺ 773.4059; found, 773.4193.

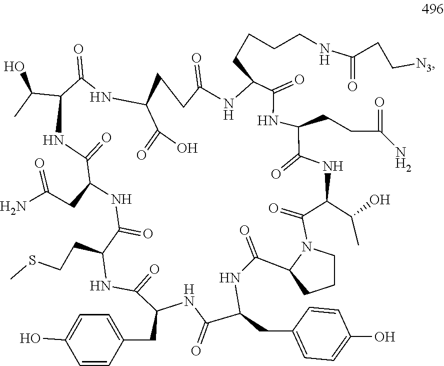 [SMILES: CSCC[C@@H]1NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC(=O)[C@H](CCCCNC(=O)CCN=[N+]=[N-])NC(=O)CC[C@@H](C(=O)O)NC(=O)[C@H]([C@@H](C)O)NC(=O)[C@H](CC(N)=O)NC1=O]

5.36 g, 96.3% pure; MS, C₅₉H₈₅N₁₆O₁₉S [M+H]⁺ 1353.5898; found, 1353.6155.

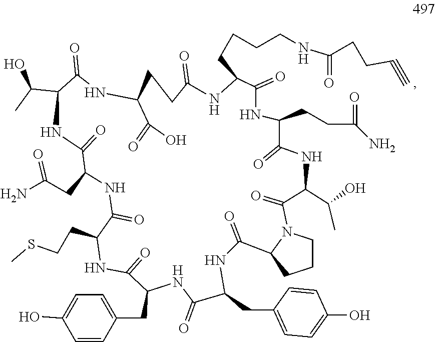 [SMILES: C#CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

5.39 g, 95.8% pure; MS, C₆₁H₈₆N₁₃O₁₉S [M+H]⁺ 1336.5884; found, 1336.6138.

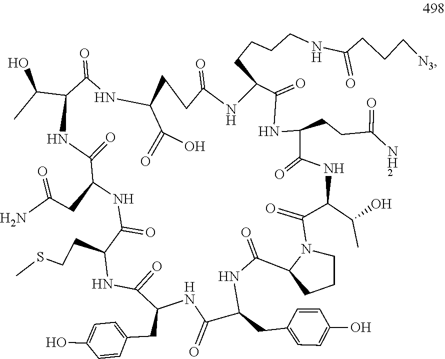 [SMILES: CSCC[C@@H]1NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC(=O)[C@H](CCCCNC(=O)CCCN=[N+]=[N-])NC(=O)CC[C@@H](C(=O)O)NC(=O)[C@H]([C@@H](C)O)NC(=O)[C@H](CC(N)=O)NC1=O]

5.23 g, 96.8% pure; MS, C₆₀H₈₇N₁₆O₁₉S [M+H]⁺ 1367.6055; found, 1367.6303.

### Example 341. Synthesis of benzyl N²-((benzyloxy)carbonyl)-N⁶-(tert-butoxycarbonyl)-L-lysyl-L-alaninate

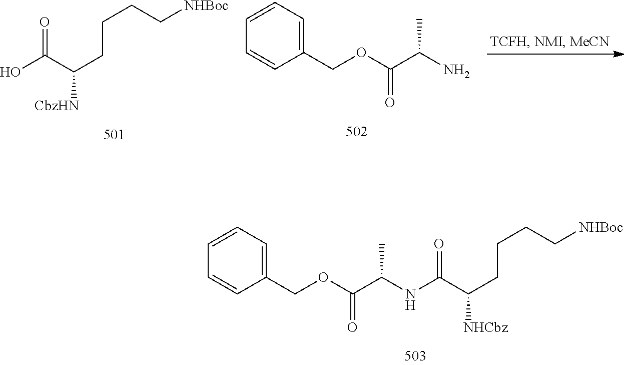 [SMILES: CC(C)(C)OC(=O)NCCCC[C@H](NC(=O)OCc1ccccc1)C(=O)O ; C[C@H](N)C(=O)OCc1ccccc1 ; C[C@H](NC(=O)[C@H](CCCCNC(=O)OC(C)(C)C)NC(=O)OCc1ccccc1)C(=O)OCc1ccccc1]

To a mixture of compound 501 (5.6 g, 7.6 mmol), compound 502 (1.7 g, 7.6 mmol) and N-methyl-imidazole (NMI) (2.0 g, 24.4 mmol) in DCM (80 mL) was added N,N,N′,N′-tetramethylchloro-formamidinium hexafluorophosphate (TCFH) (2.8 g, 9.9 mmol) at room temperature. The mixture was stirred for 2 h. The mixture was diluted with DCM (100 mL) and washed with brine (200 mL*3). The organic layer was dried and concentrated under vacuum. The residue was purified by flash column to give compound 503, 3.27 g as a colorless oil, 79.4% yield. MS-ESI (m/z): [M+H]⁺ calcd for: C₂₉H₄₀N₃0₇, 542.29; found: 542.50.

### Example 342. Synthesis of N⁶-(tert-butoxycarbonyl)-L-lysyl-L-alanine, 504

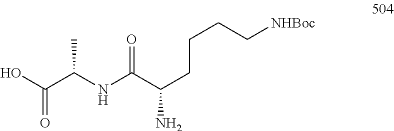 [SMILES: C[C@H](NC(=O)[C@@H](N)CCCCNC(=O)OC(C)(C)C)C(=O)O]

A mixture of compound 503 (2.0 g, 3.69 mmol) and Pd/C (300 mg) in IPA (30 mL) was stirred at room temperature under H₂ for 16 h. The mixture was filtered. The filtrate was concentrated under vacuum to give compound 504, 1.13 g (95% yield), as a colorless oil. MS-ESI (m/z): [M+H]⁺ calcd for: C₁₄H₂₈N₃O₅, 318.203; found: 318.286.

### Example 343. Synthesis of 4,4′-((2-((3aR,4R,7S,7aS)-1,3-dioxo-1,3,3a,4,7,7a-hexahydro-2H-4,7-epoxyisoindol-2-yl)-3-((3aR,4S,7R,7aS)-1,3-dioxo-1,3,3a,4,7,7a-hexahydro-2H-4,7-epoxyisoindol-2-yl)succinyl)bis(azanediyl))dibutyric acid, 505

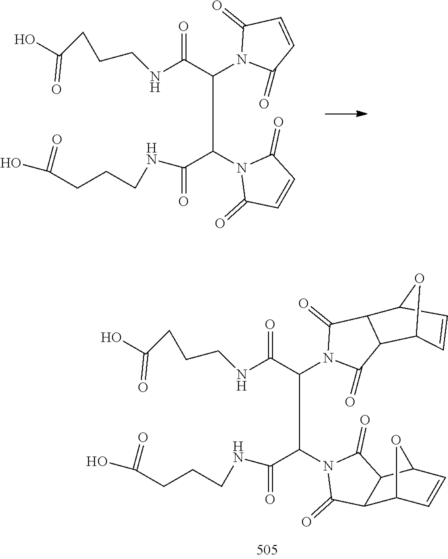 [SMILES: O=C(O)CCCNC(=O)C(C(C(=O)NCCCC(=O)O)N1C(=O)C=CC1=O)N1C(=O)C=CC1=O ; O=C(O)CCCNC(=O)C(C(C(=O)NCCCC(=O)O)N1C(=O)C2C3C=CC(O3)C2C1=O)N1C(=O)C2C3C=CC(O3)C2C1=O]

4,4′-((2,3-bis(2,5-dioxo-2,5-dihydro-1H-pyrrol-1-yl)succinyl)bis(azanediyl))dibutyric acid (25 g, 52.28 mmol) in a mixture of dry THF (160 m₁) and furan (35 m₁) was heated at 150° C. in a sealed 1 L stainless steel reactor. After stirred for 5 hours, the reaction mixture was cooled down to RT, concentrated in vacuo, purified on SiO₂ chromatography, and eluted with MeOH/DCM (1;4) containing 0.1% HOAc. The eluted fractions containing the product was pooled and concentrated to afford the title compound 29.54 g (92% yield). MS-ESI (m/z): [M+H]⁺ calcd for: C₂₈H₃₁N₄O₁₂, 615.1939; found: 615.2095.

### Example 344. Synthesis of bis(2,5-dioxopyrrolidin-1-yl) 4,4′-((2-((3aR,4R,7S,7aS)-1,3-dioxo-1,3,3a,4,7,7a-hexahydro-2H-4,7-epoxyisoindol-2-yl)-3-((3aR,4S,7R,7aS)-1,3-dioxo-1,3,3a,4,7,7a-hexahydro-2H-4,7-epoxyisoindol-2-yl)succinyl)bis(azanediyl))dibutyrate, 506

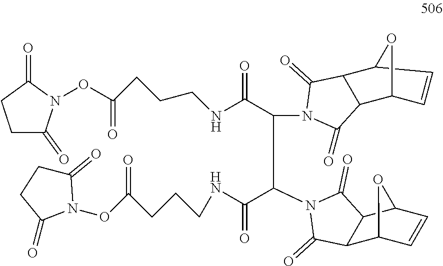 [SMILES: O=C(CCCNC(=O)C(C(C(=O)NCCCC(=O)ON1C(=O)CCC1=O)N1C(=O)C2C3C=CC(O3)C2C1=O)N1C(=O)C2C3C=CC(O3)C2C1=O)ON1C(=O)CCC1=O]

A mixture of compound 505 (2.50 g, 4.07 mmol), EHS (1.07 g, 10.19 mmol) and EDC (4.52 g, 23.31 mmol) in dry DMA (60 m₁) was stirred for 6 hours, concentrated and purified on SiO₂ column with elution of EtOAc/DCM (1:6) to afford the title compound, 2.86 g (87% yield). MS-ESI (m/z): [M+H]⁺ calcd for: C₃₆H₃₇N₆O₁₆, 809.2267; found: 809.2415.

### Example 345. Synthesis of (2S,5S,22S,25S)-5,22-bis(4-((tert-butoxycarbonyl)amino)-butyl)-13-((3aR,4R,7S,7aS)-1,3-dioxo-1,3,3a,4,7,7a-hexahydro-2H-4,7-epoxyisoindol-2-yl)-14-((3aR,4S,7R,7aS)-1,3-dioxo-1,3,3a,4,7,7a-hexahydro-2H-4,7-epoxyisoindol-2-yl)-2,25-dimethyl-4,7,12,15,20,23-hexaoxo-3,6,11,16,21,24-hexaazahexacosanedioic acid, 507

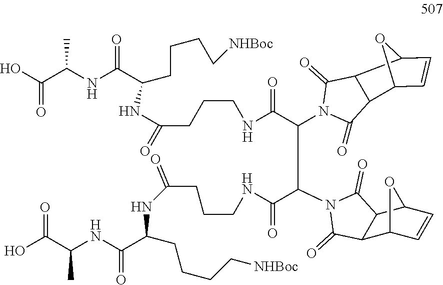 [SMILES: C[C@H](NC(=O)[C@H](CCCCNC(=O)OC(C)(C)C)NC(=O)CCCNC(=O)C(C(C(=O)NCCCC(=O)N[C@@H](CCCCNC(=O)OC(C)(C)C)C(=O)N[C@@H](C)C(=O)O)N1C(=O)C2C3C=CC(O3)C2C1=O)N1C(=O)C2C3C=CC(O3)C2C1=O)C(=O)O]

Compound 504 (750 mg, 2.36 mmol) and compound 506 (1911 mg, 2.36 mmol) in DMF (30 mL) was added DIPEA (89 mg, 0.12 m₁, 0.70 mmol) at 0° C. The mixture was then stirred for 2 h at RT, concentrated and purified by Prep-HPLC with elution of water/CH₃CN (10% CH₃CN to 60% CH₃CN in 45 min) to give compound 507, 1802 mg (63% yield), as a white solid after lyophilization, MS-ESI (m/z): [M+H]⁺ calcd for: C₅₆H₈₁N₁₀O₂₀, 1213.5629; found:1213.5985.

### Example 346. Synthesis of (2S,5S,22S,25S)-5,22-bis(4-aminobutyl)-13-((3aR,4R,7S,7aS)-1,3-dioxo-1,3,3a,4,7,7a-hexahydro-2H-4,7-epoxyisoindol-2-yl)-14-((3aR,4S,7R,7aS)-1,3-dioxo-1,3,3a,4,7,7a-hexahydro-2H-4,7-epoxyisoindol-2-yl)-2,25-dimethyl-4,7,12,15,20,23-hexaoxo-3,6,11,16,21,24-hexaazahexacosanedioic acid, 508

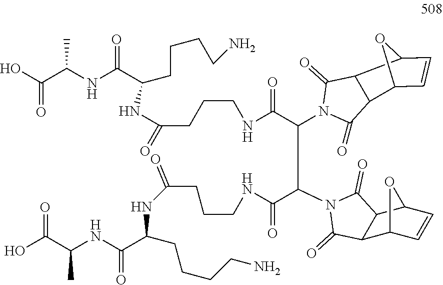 [SMILES: C[C@H](NC(=O)[C@H](CCCCN)NC(=O)CCCNC(=O)C(C(C(=O)NCCCC(=O)N[C@@H](CCCCN)C(=O)N[C@@H](C)C(=O)O)N1C(=O)C2C3C=CC(O3)C2C1=O)N1C(=O)C2C3C=CC(O3)C2C1=O)C(=O)O]

Compound 507 (1.75 g, 1.44 mmol) in DCM (10 m₁) was added TFA (3 m₁). The mixture was stirred for 30 min, diluted with DMF (5 mL) and toluene (10 mL), evaporated and dried with oil pump to afford the title compound in TFA salt (1.75 g, 100% yield). MS-ESI (m/z): [M+H]⁺ calcd for: C₄₆H₆₅N₁₀O₁₆, 1013.458; found: 1013.4685.

### Example 347. Synthesis of 2,5-dioxopyrrolidin-1-yl pent-4-enoate, 509

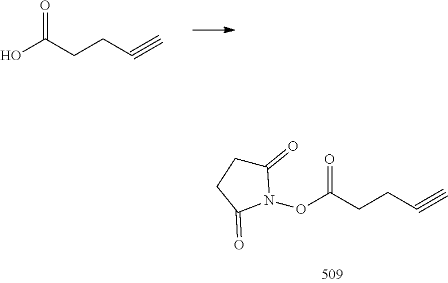

3-Vinyl propanoic acid (10.01 g, 100.05 mmol) and NHS (12.65 g, 109.97 mmol) in DCM (250 m₁) was added EDC (38.28 g, 198.34 mmol). The mixture was stirred for 6 h, evaporated and purified on SiO₂ column with elution of EtOAc/hexane (1:4) to afford the title compound, 15.98 g (81% yield). MS-ESI (m/z): [M+H]⁺ calcd for: C₉H₁₂NO₄, 198.077; found: 198.085.

### Example 348. Synthesis of (2S,5S,22S,25S)-13-((3aR,4R,7S,7aS)-1,3-dioxo-1,3,3a,4,7,7a-hexahydro-2H-4,7-epoxyisoindol-2-yl)-14-((3aR,4S,7R,7aS)-1,3-dioxo-1,3,3a,4,7,7a-hexahydro-2H-4,7-epoxyisoindol-2-yl)-2,25-dimethyl-4,7,12,15,20,23-hexaoxo-5,22-bis(4-(pent-4-ynamido)butyl)-3,6,11,16,21,24-hexaazahexacosanedioic acid, 510

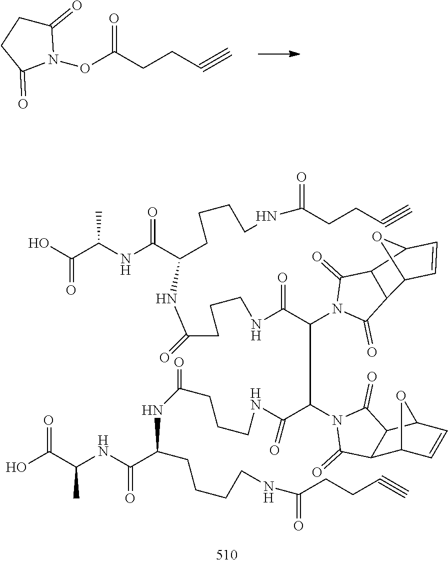 [SMILES: C#CCCC(=O)ON1C(=O)CCC1=O ; C#CCCC(=O)NCCCC[C@H](NC(=O)CCCNC(=O)C(C(C(=O)NCCCC(=O)N[C@@H](CCCCNC(=O)CCC#C)C(=O)N[C@@H](C)C(=O)O)N1C(=O)C2C3C=CC(O3)C2C1=O)N1C(=O)C2C3C=CC(O3)C2C1=O)C(=O)N[C@@H](C)C(=O)O]

Compound 508 (850 mg, 0.685 mmol) and compound 509 (395 mg, 2.00 mmol) in DMF (10 m₁) was added DIPEA (0.35 m₁, 2.00 mmol). The mixture was stirred for 2 h, evaporated and purified on SiO₂ column with elution of MeOH/DCM (1:4) containing 0.1% HOAc to afford the title compound, 538 mg (67% yield). MS-ESI (m/z): [M+H]⁺ calcd for: C₅₆H₇₃N₁₀O₁₈, 1173.510; found: 1173.655.

### Example 349. Synthesis of (9H-fluoren-9-yl)methyl ((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)methyl)amino)-1-oxopropan-2-yl)carbamate, 513

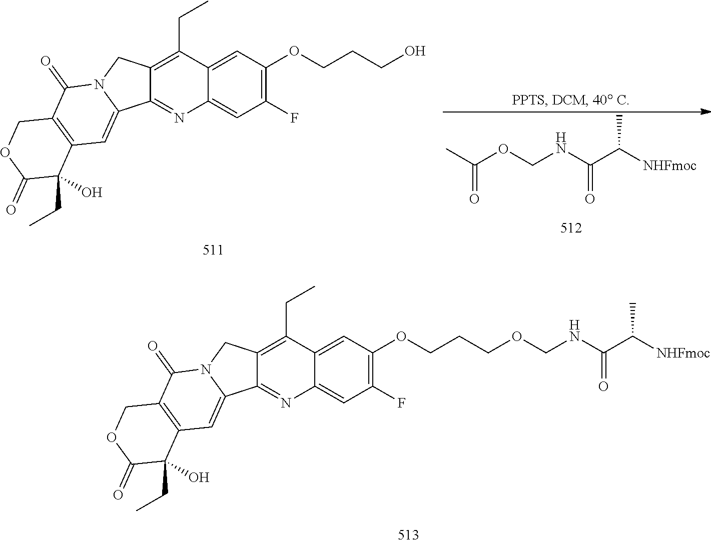

A mixture of(S)-4,11-diethyl-8-fluoro-4-hydroxy-9-(3-hydroxypropoxy)-1,12-dihydro-14H-pyrano[3′,4′:6,7]indolizino[1,2-b]quinoline-3,14(4H)-dione 511(400 mg, 0.853 mmol), (S)-(2-((((9H-fluoren-9-yl)methoxy)carbonyl)amino)propanamido)methyl acetate 512 (330 mg, 0.863 mmol) and PPTS (22 mg, 0.085 mmol) in toluene (30 mL) was stirred at 100° C. for overnight. After LC-MS showed the reaction was completed, the mixture was concentrated under vacuum, and purified by flash column to give the title compound 513, 412 mg (61% yield), as an off-white solid. MS-ESI (m/z): [M+H]⁺ calcd for: C₄₄H₄₄FN₄09, 791.3093; found: 791.3185.

### Example 350. Synthesis of (S)-2-amino-N-((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)methyl)propanamide

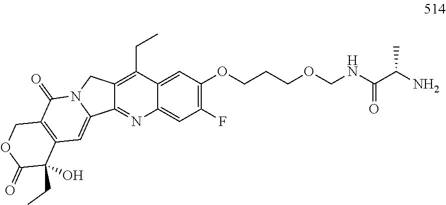 [SMILES: CCc1c2c(nc3cc(F)c(OCCCOCNC(=O)[C@H](C)N)cc13)-c1cc3c(c(=O)n1C2)COC(=O)[C@]3(O)CC]

To a mixture of compound 513 (170 mg, 0.215 mmol) in DMF (5 mL) was added piperidine (50 mg, 0.577 mmol). After stirred for 1 h and LC-MS showed the reaction was completed, the mixture was concentrated under vacuum to give crude product compound 514, (138 mg as a gum, >100% yield), which was used directly to the next step without further purification. MS-ESI (m/z): [M+H]⁺ calcd for: C₂₉H₃₄FN₄07, 569.24; found: 569.25.

### Example 351. Synthesis of benzyl N²-((benzyloxy)carbonyl)-N⁶-(tert-butoxycarbonyl)-L-lysyl-L-valinate

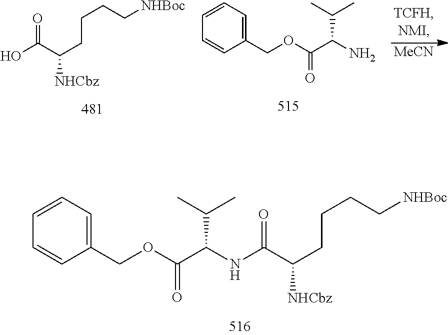 [SMILES: "MeCN" ; CC(C)(C)OC(=O)NCCCC[C@H](NC(=O)OCc1ccccc1)C(=O)O ; CC(C)[C@H](N)C(=O)OCc1ccccc1 ; CC(C)[C@H](NC(=O)[C@H](CCCCNC(=O)OC(C)(C)C)NC(=O)OCc1ccccc1)C(=O)OCc1ccccc1]

To a mixture of compound 481 (5.50 g, 14.47 mmol), benzyl L-valinate 515 (3.00 g, 14.47 mmol) and N-methylimidazole (NMI) (2.00 g, 24.4 mmol) in DCM (80 mL) was added N,N,N′,N′-tetramethylchloro-formamidinium hexafluorophosphate (TCFH) (5.61 g, 20.00 mmol) at room temperature. The mixture was stirred for 2h, diluted with DCM (100 mL) and washed with brine (200 mL*3). The organic layer was dried over Na₂SO₄, filtered, concentrated under vacuum, and purified by flash column to give the title compound 516, 6.75 g (82% yield) as a colorless oil. MS-ESI (m/z): [M+H]⁺ calcd for: C₃₁H₄₄N₃O₇, 570.318; found: 570.45.

### Example 352. Synthesis of N⁶-N6-(tert-butoxycarbonyl)-L-lysyl-L-valine, 517

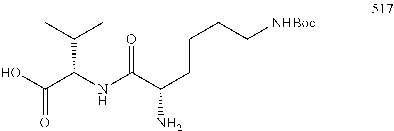 [SMILES: CC(C)[C@H](NC(=O)[C@@H](N)CCCCNC(=O)OC(C)(C)C)C(=O)O]

A mixture of compound 516 (2.50 g, 4.39 mmol) and Pd/C (300 mg) in IPA (30 mL) was stirred at room temperature under H₂ for 16 h. The mixture was filtered and concentrated under vacuum to give compound 517, 1.48 g (96% yield), as a colorless oil. MS-ESI (m/z): [M+H]⁺ calcd for: C₁₆H₃₂N₃O₅, 346.234; found: 346.245.

### Example 353. Synthesis of compound 518

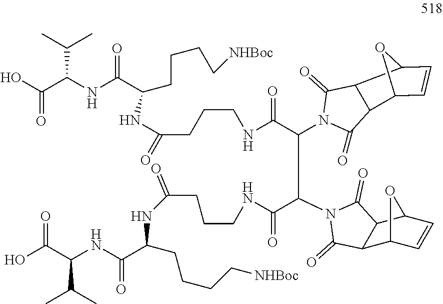 [SMILES: CC(C)[C@H](NC(=O)[C@H](CCCCNC(=O)OC(C)(C)C)NC(=O)CCCNC(=O)C(C(C(=O)NCCCC(=O)N[C@@H](CCCCNC(=O)OC(C)(C)C)C(=O)N[C@H](C(=O)O)C(C)C)N1C(=O)C2C3C=CC(O3)C2C1=O)N1C(=O)C2C3C=CC(O3)C2C1=O)C(=O)O]

Compound 517 (815 mg, 2.35 mmol) and compound 586 (1910 mg, 2.35 mmol) in DMF (30 mL) was added DIPEA (89 mg, 0.12 m₁, 0.70 mmol) at 0° C. The mixture was then stirred for 2 h at RT, concentrated and purified by Prep-HPLC with elution of water/CH₃CN (10% CH₃CN to 60% CH₃CN in 45 min) to give compound 518, 2.027 g (68% yield), as a white solid after lyophilization, MS-ESI (m/z): [M+H]⁺ calcd for: C₆₀H₈₉N₁₀O₂₀, 1269.6255; found: 1269.6335.

### Example 354. Synthesis of compound 519

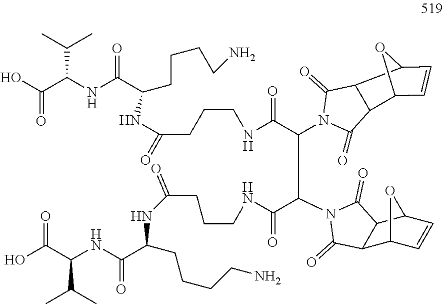 [SMILES: CC(C)[C@H](NC(=O)[C@H](CCCCN)NC(=O)CCCNC(=O)C(C(C(=O)NCCCC(=O)N[C@@H](CCCCN)C(=O)N[C@H](C(=O)O)C(C)C)N1C(=O)C2C3C=CC(O3)C2C1=O)N1C(=O)C2C3C=CC(O3)C2C1=O)C(=O)O]

Compound 518 (1.05 g, 0.827 mmol) in DCM (10 m₁) was added TFA (3 m₁). The mixture was stirred for 30 min, diluted with DMF (5 mL) and toluene (10 mL), evaporated and dried with oil pump to afford the compound 519 as TFA salt (1.02 g, >100% yield). MS-ESI (m/z): [M+H]⁺ calcd for: C₅₀H₇₃N₁₀O₁₆, 1069.5207; found: 1069.5305.

### Example 355. Synthesis of (2S,5S,22S,25S)-13-((3aR,4R,7S,7aS)-1,3-dioxo-1,3,3a,4,7,7a-hexahydro-2H-4,7-epoxyisoindol-2-yl)-14-((3aR,4S,7R,7aS)-1,3-dioxo-1,3,3a,4,7,7a-hexahydro-2H-4,7-epoxyisoindol-2-yl)-2,25-diisopropyl-4,7,12,15,20,23-hexaoxo-5,22-bis(4-(pent-4-ynamido)butyl)-3,6,11,16,21,24-hexaazahexacosanedioic acid, 520

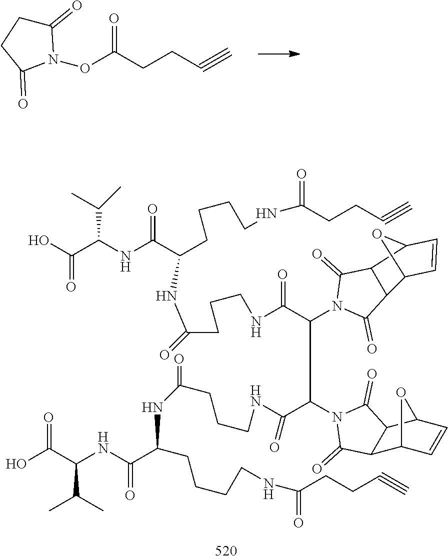 [SMILES: C#CCCC(=O)ON1C(=O)CCC1=O ; C#CCCC(=O)NCCCC[C@H](NC(=O)CCCNC(=O)C(C(C(=O)NCCCC(=O)N[C@@H](CCCCNC(=O)CCC#C)C(=O)N[C@H](C(=O)O)C(C)C)N1C(=O)C2C3C=CC(O3)C2C1=O)N1C(=O)C2C3C=CC(O3)C2C1=O)C(=O)N[C@H](C(=O)O)C(C)C]

Compound 519 (740 mg, 0.692 mmol) and compound 509 (395 mg, 2.00 mmol) in DMF (10 m₁) was added DIPEA (0.35 m₁, 2.00 mmol). The mixture was stirred for 2 h, evaporated and purified on SiO₂ column with elution of MeOH/DCM (1:4) containing 0.1% HOAc to afford the title compound, 586 mg (69% yield). MS-ESI (m/z): [M+H]⁺ calcd for: C₆₀H₈₁N₁₀O₁₈, 1229.573; found: 1229.589.

### Example 356. Synthesis of N,N⁴-bis((8S,11S,14S)-1-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)-11-isopropyl-8-methyl-7,10,13,16-tetraoxo-14-(4-(pent-4-ynamido)butyl)-4-oxa-6,9,12,15-tetraazanonadecan-19-yl)-2-((3aR,4R,7S,7aS)-1,3-dioxo-1,3,3a,4,7,7a-hexahydro-2H-4,7-epoxyisoindol-2-yl)-3-((3aR,4S,7R,7aS)-1,3-dioxo-1,3,3a,4,7,7a-hexahydro-2H-4,7-epoxyisoindol-2-yl)succinamide, 521

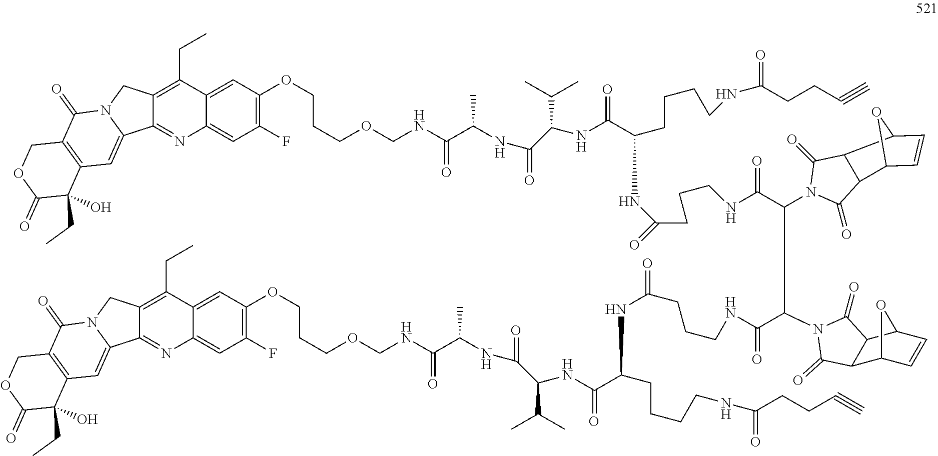 [SMILES: C#CCCC(=O)NCCCC[C@H](NC(=O)CCCNC(=O)C(C(C(=O)NCCCC(=O)N[C@@H](CCCCNC(=O)CCC#C)C(=O)N[C@H](C(=O)N[C@@H](C)C(=O)NCOCCCOc1cc2c(CC)c3c(nc2cc1F)-c1cc2c(c(=O)n1C3)COC(=O)[C@]2(O)CC)C(C)C)N1C(=O)C2C3C=CC(O3)C2C1=O)N1C(=O)C2C3C=CC(O3)C2C1=O)C(=O)N[C@H](C(=O)N[C@@H](C)C(=O)NCOCCCOc1cc2c(CC)c3c(nc2cc1F)-c1cc2c(c(=O)n1C3)COC(=O)[C@]2(O)CC)C(C)C]

To a mixture of compound 520 (406 mg, 0.330 mmol) and compound 514 (410 mg, 0.722 mmol) in DMA (20 m₁) was added EDC (300 mg, 1.554 mmol). The mixture was stirred for 6 h, evaporated and purified on SiO₂ column with elution of EtOAc/DCM (1:4) to afford the title compound, 512 mg (67% yield). MS-ESI (m/z): [M+H]⁺ calcd for: C₁₁₈H₁₄₃F₂N18O₃₀, 2330.0186; found: 2330.0395.

### Example 357. Synthesis of(S)-2-((S)-2-(λ²-azaneyl)propanamido)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propanamide--2-((3aR,4R,7S,7aS)-1,3-dioxo-1,3,3a,4,7,7a-hexahydro-2H-4,7-epoxyisoindol-2-yl)-3-((3aR,4S,7R,7aS)-1,3-dioxo-1,3,3a,4,7,7a-hexahydro-2H-4,7-epoxyisoindol-2-yl)-N1,N4-bis((2S,5S,8S,11S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,5,8-trimethyl-1,4,7,10,13-pentaoxo-11-(4-(pent-4-ynamido)butyl)-3,6,9,12-tetraazahexadecan-16-yl)succinamide (1/1), 523

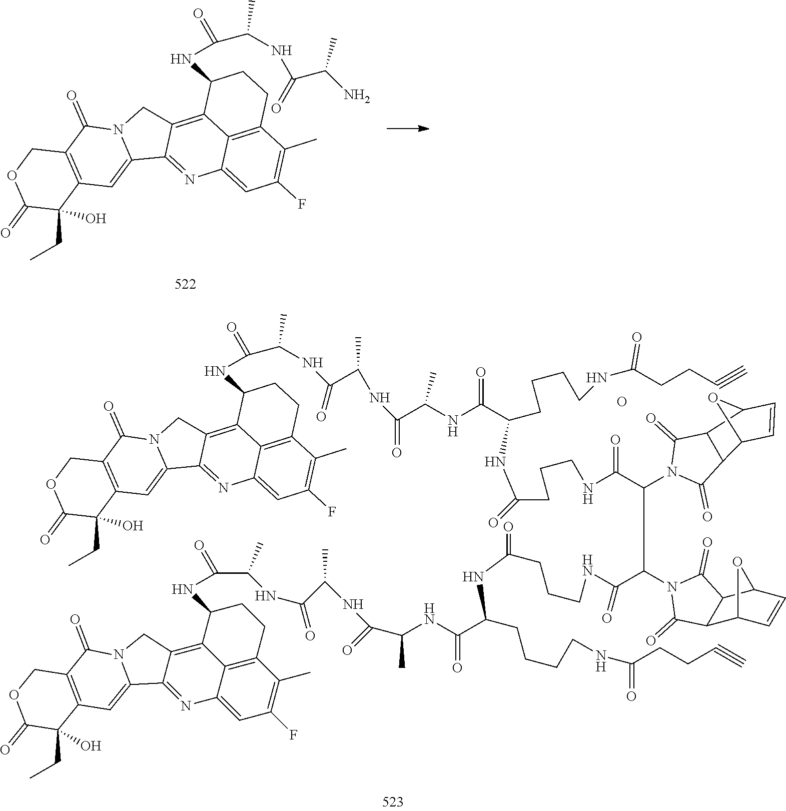

To a mixture of compound 510 (525 mg, 0.446 mmol) and (S)-2-amino-N—((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1-oxopropan-2-yl)propanamide, HCl salt (compound 522) (585 mg, 0.953 mmol) in DMA (20 m₁) was added EDC (455 mg, 2.357 mmol). The mixture was stirred for 6 h, evaporated and purified on SiO₂ column with elution of EtOAc/DCM (1:8 to 1:3) to afford the title compound, 643 mg (63% yield). MS-ESI (m/z): [M+H]⁺ calcd for: C₁₁₆H₁₃₃F₂N₂₀O₂₈, 2291.9567; found: 2291.9895.

### Example 358. Synthesis of N⁶-(tert-butoxycarbonyl)-N²-(4-((3aR,4R,7S,7aS)-1,3-dioxo-1,3,3a,4,7,7a-hexahydro-2H-4,7-epoxyisoindol-2-yl)butanoyl)-L-lysyl-L-valine

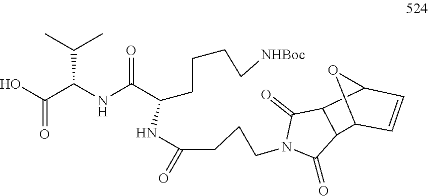 [SMILES: CC(C)[C@H](NC(=O)[C@H](CCCCNC(=O)OC(C)(C)C)NC(=O)CCCN1C(=O)C2C3C=CC(O3)C2C1=O)C(=O)O]

2,5-dioxopyrrolidin-1-yl 4-((3aR,4R,7S,7aS)-1,3-dioxo-1,3,3a,4,7,7a-hexahydro-2H-4,7-epoxyisoindol-2-yl)butanoate (822 mg, 2.36 mmol) and Compound 517 (817 mg, 2.36 mmol) in DMF (25 mL) was added DIPEA (74 mg, 0.10 m₁, 0.573 mmol) at 0° C. The mixture was then stirred at RT for 2h, concentrated and purified by SiO₂ column with elution of MeOH/DCM (1:10 to 1:6) containing 1% HOAc to give the title, 955 mg (70% yield), as a white solid, MS-ESI (m/z): [M+H]⁺ calcd for: C₂₈H₄₃N₄O₉, 579.3031; found: 579.3190.

### Example 359. Synthesis of N⁶-(tert-butoxycarbonyl)-N²-(4-((3aR,4R,7S,7aS)-1,3-dioxo-1,3,3a,4,7,7a-hexahydro-2H-4,7-epoxyisoindol-2-yl)butanoyl)-L-lysyl-L-alanine, 525

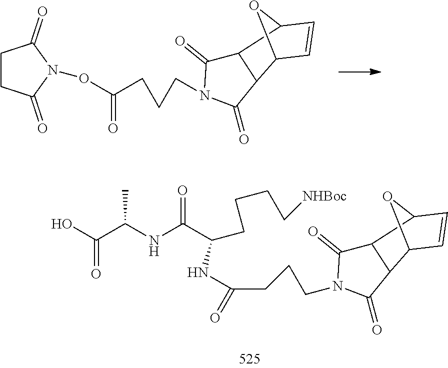 [SMILES: O=C(CCCN1C(=O)C2C3C=CC(O3)C2C1=O)ON1C(=O)CCC1=O ; C[C@H](NC(=O)[C@H](CCCCNC(=O)OC(C)(C)C)NC(=O)CCCN1C(=O)C2C3C=CC(O3)C2C1=O)C(=O)O]

2,5-dioxopyrrolidin-1-yl 4-((3aR,4R,7S,7aS)-1,3-dioxo-1,3,3a,4,7,7a-hexahydro-2H-4,7-epoxyisoindol-2-yl)butanoate (822 mg, 2.36 mmol) and Compound 584 (750 mg, 2.36 mmol) in DMF (25 mL) was added DIPEA (74 mg, 0.10 m₁, 0.573 mmol) at 0° C. The mixture was then stirred at RT for 2h, concentrated and purified by SiO₂ column with elution of MeOH/DCM (1:10 to 1:6) containing 1% HOAc to give the title, 943 mg (72% yield), as a white solid, MS-ESI (m/z): [M+H]⁺ calcd for: C₂₆H₃₉N₄O₉, 551.2718; found:551.2795.

### Example 360. Synthesis of (4-((3aR,4R,7S,7aS)-1,3-dioxo-1,3,3a,4,7,7a-hexahydro-2H-4,7-epoxyisoindol-2-yl)butanoyl)-L-lysyl-L-valine

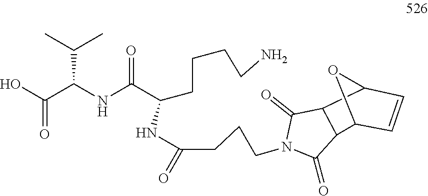 [SMILES: CC(C)[C@H](NC(=O)[C@H](CCCCN)NC(=O)CCCN1C(=O)C2C3C=CC(O3)C2C1=O)C(=O)O]

Compound 524 (950 mg, 1.642 mmol) in dioxane (8 m₁) on ice bath was added HCl (cone. 36%, 2 m₁). The mixture was stirred for 40 min, diluted with dioxane (10 mL) and toluene (10 mL), evaporated and co-evaporated with dioxane/toluene (1:1 10 m₁) and dried with oil pump to afford the title compound 606 as HCl salt (795 mg, >100% yield), which was used directly for the next step without further purification. MS-ESI (m/z): [M+H]⁺ calcd for: C₂₃H₃₅N₄O₇, 479.2506; found: 479.2750.

### Example 361. Synthesis of(4-((3aR,4R,7S,7aS)-1,3-dioxo-1,3,3a,4,7,7a-hexahydro-2H-4,7-epoxyisoindol-2-yl)butanoyl)-L-lysyl-L-alanine

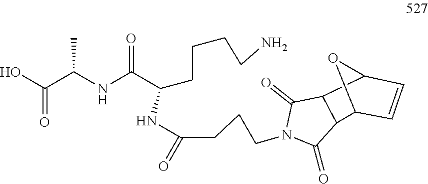 [SMILES: C[C@H](NC(=O)[C@H](CCCCN)NC(=O)CCCN1C(=O)C2C3C=CC(O3)C2C1=O)C(=O)O]

Compound 525 (930 mg, 1.70 mmol) in dioxane (8 m₁) on ice bath was added HCl (cone. 36%, 2 m₁). The mixture was stirred for 40 min, diluted with dioxane (10 mL) and toluene (10 mL), evaporated and co-evaporated with dioxane/toluene (1:1 10 m₁) and dried with oil pump to afford the title compound 606 as HCl salt (770 mg, >100% yield), which was used directly for the next step without further purification. MS-ESI (m/z): [M+H]⁺ calcd for: C₂₁H₃₁N₄O₇, 451.2193; found: 451.2385.

### Example 362. Synthesis of N²-(4-((3aR,4R,7S,7aS)-1,3-dioxo-1,3,3a,4,7,7a-hexahydro-2H-4,7-epoxyisoindol-2-yl)butanoyl)-N⁶-(pent-4-ynoyl)-L-lysyl-L-valine, 528

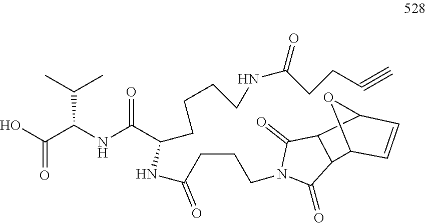 [SMILES: C#CCCC(=O)NCCCC[C@H](NC(=O)CCCN1C(=O)C2C3C=CC(O3)C2C1=O)C(=O)N[C@H](C(=O)O)C(C)C]

Compound 526 (783 mg, 1.52 mmol) and compound 509 (395 mg, 2.00 mmol) in DMF (10 m₁) was added DIPEA (0.30 m₁, 1.72 mmol). The mixture was stirred for 2 h, evaporated and purified on SiO₂ column with elution of MeOH/DCM (1:4) containing 0.1% HOAc to afford the title compound, 602 mg (71% yield). MS-ESI (m/z): [M+H]⁺ calcd for: C₂₈H₃₉N₄O₈, 559.298; found: 559.435.

### Example 363. Synthesis of(S)—N—((S)-1-(((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)methyl)amino)-1-oxopropan-2-yl)amino)-3-methyl-1-oxobutan-2-yl)-2-(4-((3aR,4R,7S,7aS)-1,3-dioxo-1,3,3a,4,7,7a-hexahydro-2H-4,7-epoxyisoindol-2-yl)butanamido)-6-(pent-4-ynamido)hexanamide, 529

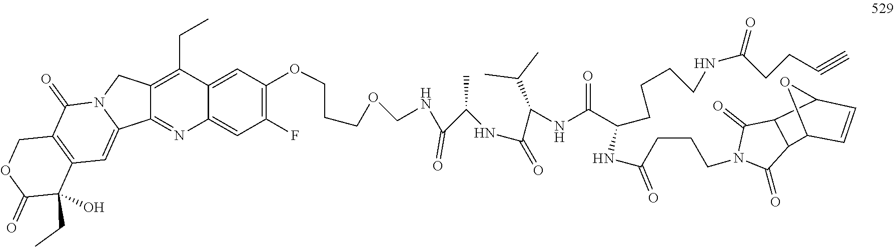 [SMILES: C#CCCC(=O)NCCCC[C@H](NC(=O)CCCN1C(=O)C2C3C=CC(O3)C2C1=O)C(=O)N[C@H](C(=O)N[C@@H](C)C(=O)NCOCCCOc1cc2c(CC)c3c(nc2cc1F)-c1cc2c(c(=O)n1C3)COC(=O)[C@]2(O)CC)C(C)C]

To a mixture of compound 528 (422 mg, 0.755 mmol) and compound 514 (431 mg, 0.758 mmol) in DMA (20 m₁) was added EDC (485 mg, 2.513 mmol). The mixture was stirred for 6 h, evaporated and purified on SiO₂ column with elution of EtOAc/DCM (1:4) to afford the title compound, 543 mg (65% yield). MS-ESI (m/z): [M+H]⁺ calcd for: C₅₇H₇₀FN₅O₁₄, 1181.500; found: 1181.595.

### Example 364. Synthesis of N²-(4-((3aR,4R,7S,7aS)-1,3-dioxo-1,3,3a,4,7,7a-hexahydro-2H-4,7-epoxyisoindol-2-yl)butanoyl)-N⁶-(pent-4-ynoyl)-L-lysyl-L-alanine, 610

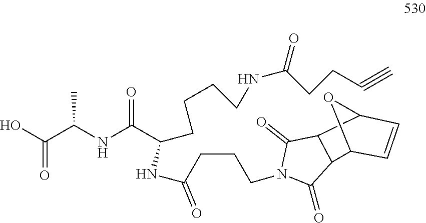 [SMILES: C#CCCC(=O)NCCCC[C@H](NC(=O)CCCN1C(=O)C2C3C=CC(O3)C2C1=O)C(=O)N[C@@H](C)C(=O)O]

Compound 527 (671 mg, 1.49 mmol) and compound 509 (395 mg, 2.00 mmol) in DMF (10 m₁) was added DIPEA (0.30 m₁, 1.72 mmol). The mixture was stirred for 2 h, evaporated and purified on SiO₂ column with elution of MeOH/DCM (1:4) containing 0.1% HOAc to afford the title compound, 602 mg (71% yield). MS-ESI (m/z): [M+H]⁺ calcd for: C₂₆H₃₅N₄O₈, 531.2456; found: 531.2535.

### Example 365. Synthesis of (S)-2-(4-((3aR,4R,7S,7aS)-1,3-dioxo-1,3,3a,4,7,7a-hexahydro-2H-4,7-epoxyisoindol-2-yl)butanamido)-N—((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1-oxopropan-2-yl)amino)-1-oxopropan-2-yl)amino)-1-oxopropan-2-yl)-6-(pent-4-ynamido)hexanamide, 531

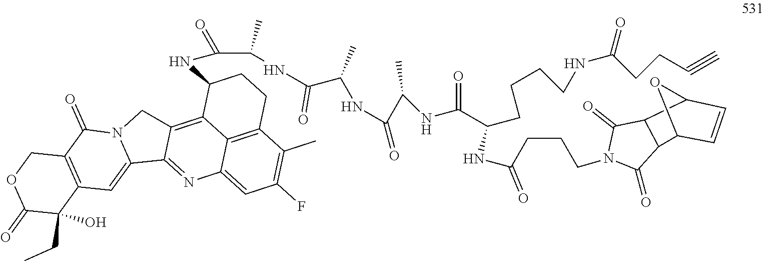 [SMILES: C#CCCC(=O)NCCCC[C@H](NC(=O)CCCN1C(=O)C2C3C=CC(O3)C2C1=O)C(=O)N[C@@H](C)C(=O)N[C@@H](C)C(=O)N[C@@H](C)C(=O)N[C@H]1CCc2c(C)c(F)cc3nc4c(c1c23)Cn1c-4cc2c(c1=O)COC(=O)[C@]2(O)CC]

To a mixture of compound 530 (558 mg, 1.052 mmol) and (S)-2-amino-N—((S)-1-(((1S,9S)-9-ethyl-5-fluoro-9-hydroxy-4-methyl-10,13-dioxo-2,3,9,10,13,15-hexahydro-1H,12H-benzo[de]pyrano-[3′,4′:6,7]-indolizino[1,2-b]quinolin-1-yl)amino)-1-oxopropan-2-yl)propanamide, HCl salt (compound 522) (620 mg, 1.010 mmol) in DMA (20 m₁) was added EDC (491 mg, 2.544 mmol). The mixture was stirred for 6 h, evaporated and purified on SiO₂ column with elution of EtOAc/DCM (1:8 to 1:3) to afford the title compound, 643 mg (63% yield). MS-ESI (m/z): [M+H]⁺ calcd for: C₅₆H₆₅FN₉O₁₃, 1090.4687; found: 1090.4858.

### Example 366. Synthesis of tert-butyl ((2S,5S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-12,12-bis((3-(((S)-1-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)carbamate, 532

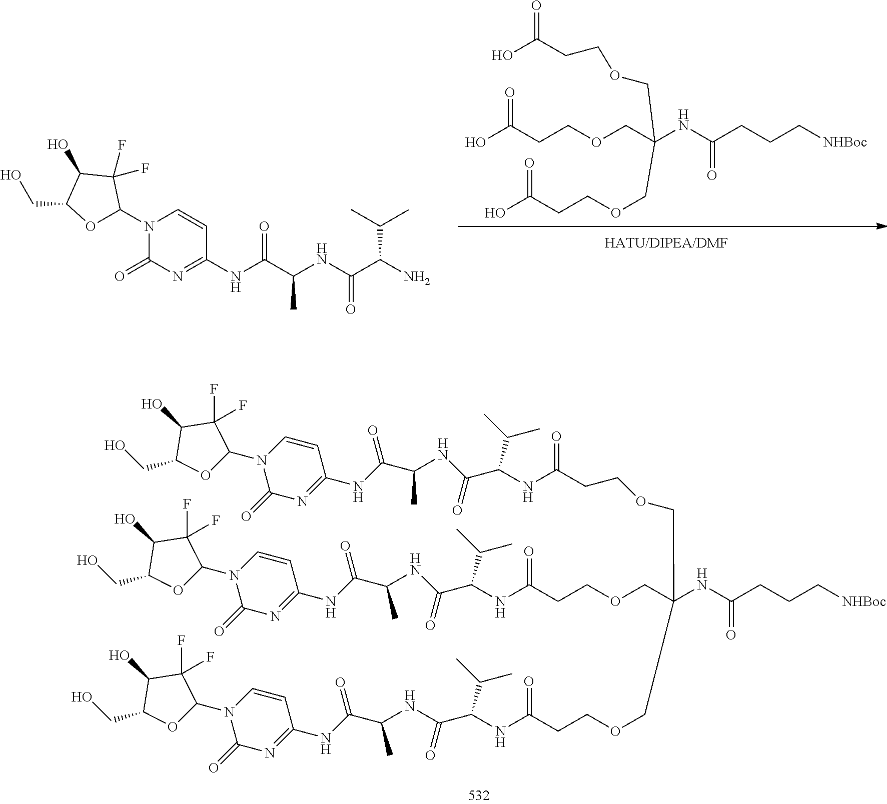

A solution of (S)-2-amino-N—((S)-1-((1-((2R,4R,5R)-3,3-difluoro-4-hydroxy-5-(hydroxymethyl)-tetrahydrofuran-2-yl)-2-oxo-1,2-dihydropyrimidin-4-yl)amino)-1-oxopropan-2-yl)-3-methyl-butanamide (2.301 g, 5.312 mmol), 11,11-bis((2-carboxyethoxy)methyl)-2,2-dimethyl-4,9-dioxo-3,13-dioxa-5,10-diazahexadecan-16-oic acid (0.927 g, 1.775 mmol), HATU (3.680 g, 9.678 mmol) and DIPEA (1.40 m₁, 8.03 mmol) in DMF (80 m₁) was stirred at RT for overnight, evaporated and purified on SiO₂ column with elution of MeOH/DCM (1:9 to 1:4) to afford the title compound, 2.097g (67% yield). MS-ESI (m/z): [M+H]⁺ calcd for: C₇₃H₁₀₈F₆N₁₇O₂₇, 1768.7506; found: 1768.7685.

### Example 367. Synthesis of ((2S,5S)-1-((1-((2R,4R,5R)-3,3-difluoro-4-hydroxy-5-(hydroxyl-methyl)tetrahydrofuran-2-yl)-2-oxo-1,2-dihydropyrimidin-4-yl)amino)-12,12-bis((3-(((S)-1-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)amine, HCl salt, 533

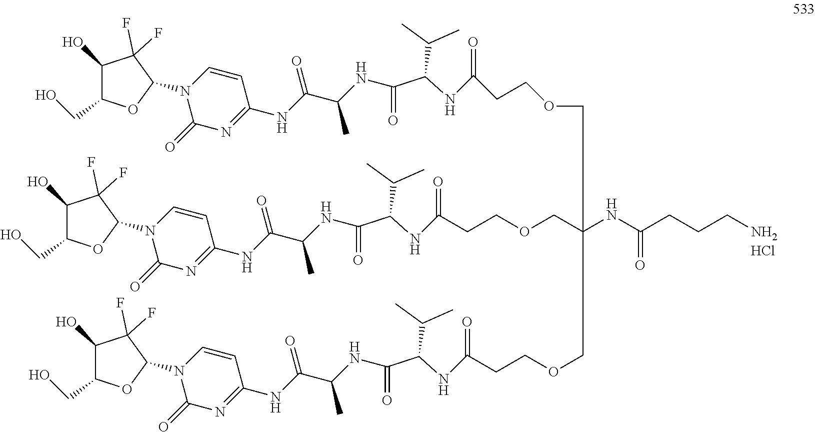 [SMILES: CC(C)[C@H](NC(=O)CCOCC(COCCC(=O)N[C@H](C(=O)N[C@@H](C)C(=O)Nc1ccn([C@@H]2O[C@H](CO)[C@@H](O)C2(F)F)c(=O)n1)C(C)C)(COCCC(=O)N[C@H](C(=O)N[C@@H](C)C(=O)Nc1ccn([C@@H]2O[C@H](CO)[C@@H](O)C2(F)F)c(=O)n1)C(C)C)NC(=O)CCCN)C(=O)N[C@@H](C)C(=O)Nc1ccn([C@@H]2O[C@H](CO)[C@@H](O)C2(F)F)c(=O)n1 ; Cl]

Compound 532 (1.051 g, 0.594 mmol) in dioxane (10 m₁) on ice batch was added HCl (36%, 3 m₁). The mixture was stirred on ice bath for 30 min, diluted with dioxane (10 m₁) and toluene (10 m₁), concentrated at 2 −8° C. in vacuo, and co-evaporated two times with dioxane (10 m₁) and toluene (10 m₁) at 2 −8° C. in vacuo, and dried with oil vacuum pump to afford the title crude product (1.022 g, >100 yield) for direct use in the next step without further purification. MS-ESI (m/z): [M+H]⁺ calcd for: C₆₈H₁₀₀F₆N₁₇O₂₅, 1668.6981; found: 1668.7125 Example 368. Synthesis of (S)-N5-((2S,5S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-12,12-bis((3-(((S)-1-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)-2-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)pentanediamide, 534. 7 3

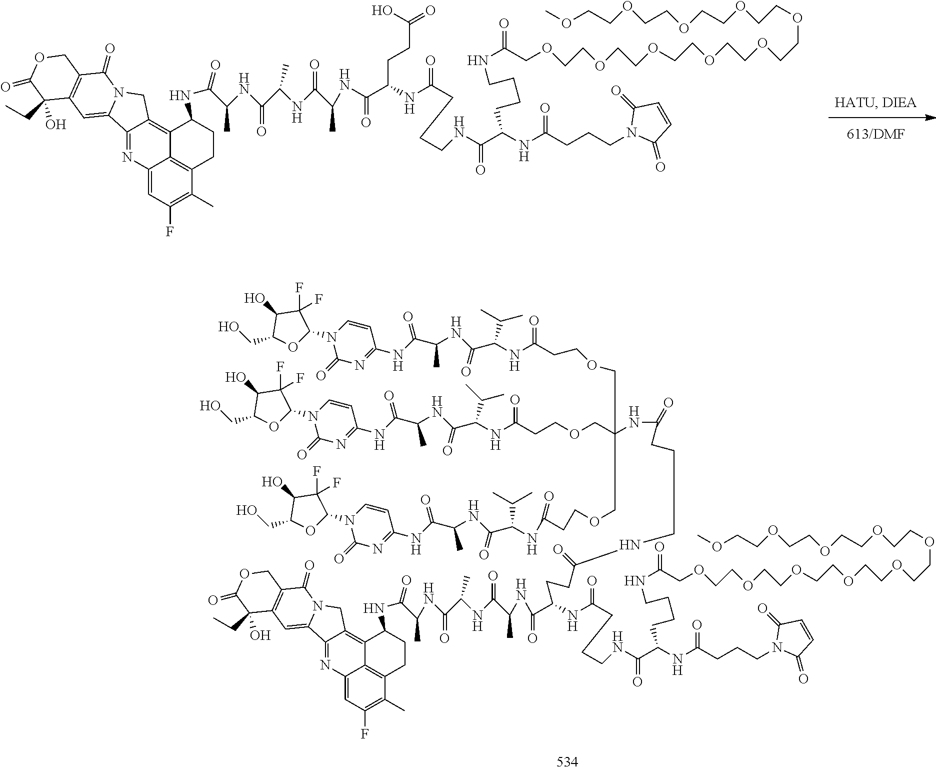

A solution of (37S,45S)-37-(4-(2,5-dioxo-2,5-dihydro-1H-pyrrol-1-yl)butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oic acid (250 mg, 0.154 mmol), compound 533 (255 mg, 0.153 mmol), HATU (186 mg, 0.489 mmol), DIPEA (0.050 m₁, 0.286 mmol) in DMF (6 m₁) was stirred for 8 h, evaporated and purified by C18 prep HPLC (50 mm×250 mm) with elution of MeOH/H2O (1:9 to 1:3 in 50 min at 20 ml/min) to afford the title compound, 341 mg (68% yield). MS-ESI (m/z): [M+H]⁺ calcd for: C₁₄₅H₂₀₈F₇N₂₈O₅₀, 3274.4483; found: 3274.4625.

### Example 369. Synthesis of (S)-N5-(4-((1,3-bis(3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propan-2-yl)amino)-4-oxobutyl)-2-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)pentanediamide, 536

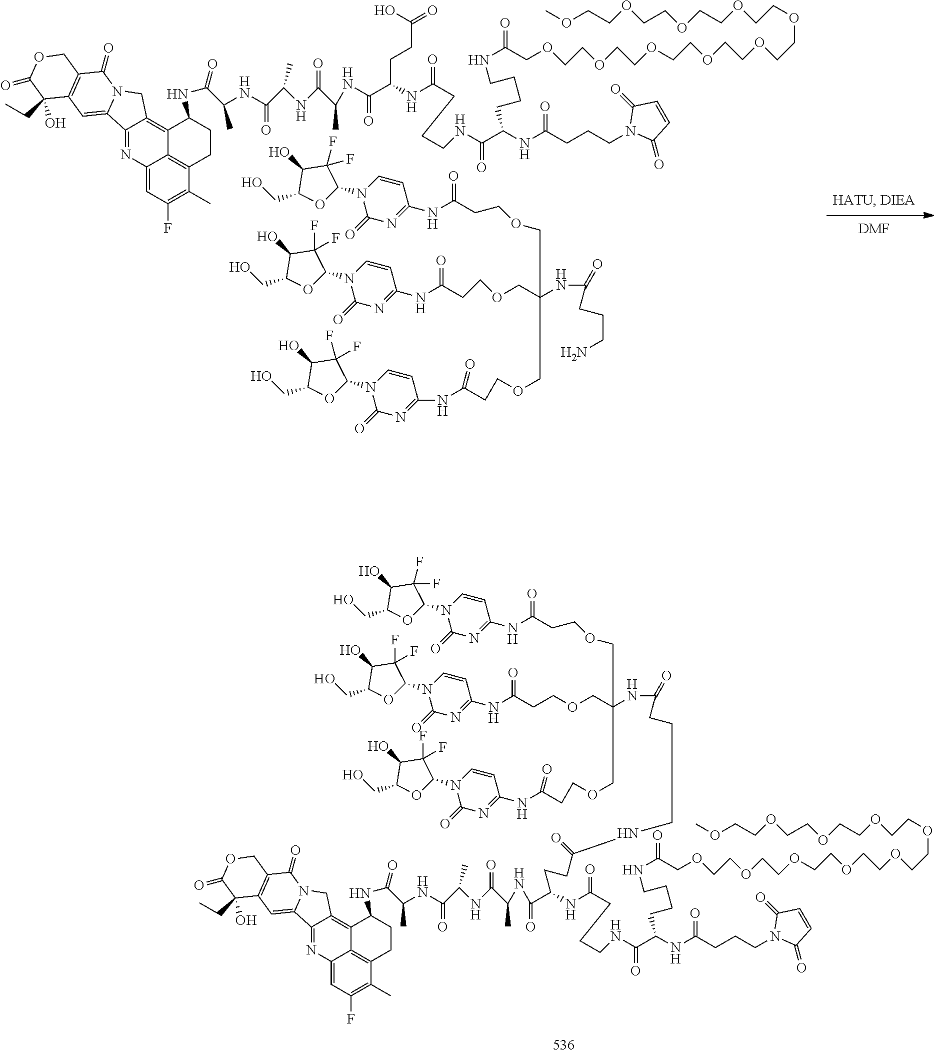

A solution of (37S,45S)-37-(4-(2,5-dioxo-2,5-dihydro-1H-pyrrol-1-yl)butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oic acid (250 mg, 0.154 mmol), 3,3′-((2-(4-aminobutanamido)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propane-1,3-diyl)bis(oxy))bis(N-(1-((2R,4R,5R)-3,3-difluoro-4-hydroxy-5-(hydroxymethyl)tetrahydrofuran-2-yl)-2-oxo-1,2-dihydropyrimidin-4-yl)propanamide) (181 mg, 0.156 mmol), HATU (183 mg, 0.481 mmol), DIPEA (0.050 m₁, 0.286 mmol) in DMF (6 m₁) was stirred for 8 h, evaporated and purified by C18 prep HPLC (50 mm×250 mm) with elution of MeOH/H₂O (1:9 to 1:3 in 50 min at 20 ml/min) to afford the title compound, 263 mg (62% yield). MS-ESI (m/z): [M+H]⁺ calcd for: C₁₂₁H₁₆₆F₇N₂₂O₄₄, 2764.1317; found: 2764.1595.

### Example 370. Synthesis of tert-butyl ((2S,5S)-1-((1-((2R,3R,4S,5R)-5-ethyl-3,4-dihydroxytetrahydrofuran-2-yl)-5-fluoro-2-oxo-1,2-dihydropyrimidin-4-yl)amino)-12,12-bis((3-(((S)-1-(((S)-1-((1-((2R,3R,4S,5R)-5-ethyl-3,4-dihydroxytetrahydrofuran-2-yl)-5-fluoro-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)carbamate, 538

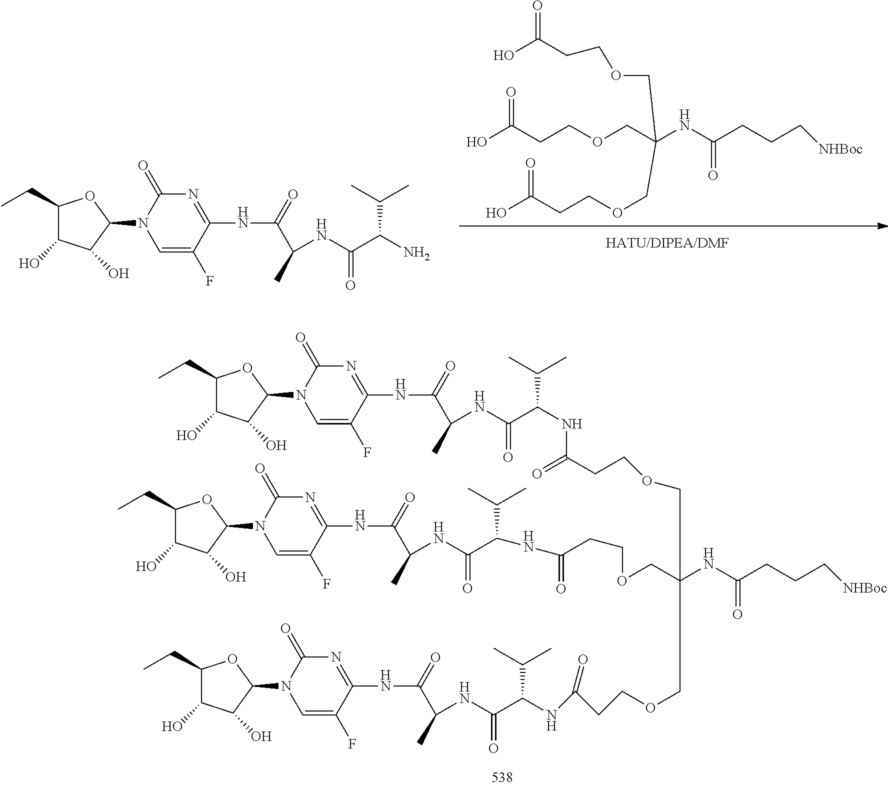

A solution of (S)-2-amino-N—((S)-1-((1-((2R,3R,4S,5R)-5-ethyl-3,4-dihydroxytetrahydrofuran-2-yl)-5-fluoro-2-oxo-1,2-dihydropyrimidin-4-yl)amino)-1-oxopropan-2-yl)-3-methylbutanamide (2.332 g, 5.433 mmol), 11,11-bis((2-carboxyethoxy)methyl)-2,2-dimethyl-4,9-dioxo-3,13-dioxa-5,10-diazahexadecan-16-oic acid (0.933 g, 1.786 mmol), HATU (3.680 g, 9.678 mmol) and DIPEA (1.40 m₁, 8.03 mmol) in DMF (80 m₁) was stirred at RT for overnight, evaporated and purified on C-18 HPLC column (50×800 mm) with elution of MeOH/H₂O (1:9 to 5:1, 90 ml/min in 45 min) to afford the title compound, 2.038g (65% yield). MS-ESI (m/z): [M+H]⁺ calcd for: C₇₆Hi ₁₇F₃N₁₇O₂₇, 1756.8258; found: 1756.8435.

### Example 371. Synthesis of ((2S,5S)-1-((1-((2R,3R,4S,5R)-5-ethyl-3,4-dihydroxytetrahydro-furan-2-yl)-5-fluoro-2-oxo-1,2-dihydropyrimidin-4-yl)amino)-12,12-bis((3-(((S)-1-(((S)-1-((1-((2R,3R,4S,5R)-5-ethyl-3,4-dihydroxytetrahydrofuran-2-yl)-5-fluoro-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)amine, HCl salt, 539

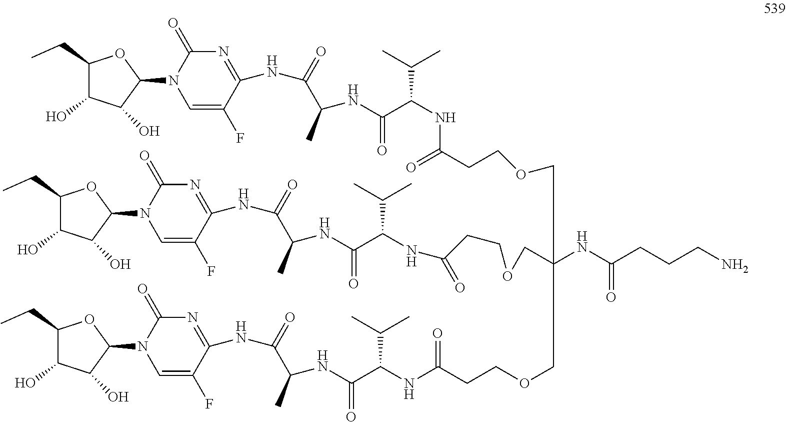 [SMILES: CC[C@H]1O[C@@H](n2cc(F)c(NC(=O)[C@H](C)NC(=O)[C@@H](NC(=O)CCOCC(COCCC(=O)N[C@H](C(=O)N[C@@H](C)C(=O)Nc3nc(=O)n([C@@H]4O[C@H](CC)[C@@H](O)[C@H]4O)cc3F)C(C)C)(COCCC(=O)N[C@H](C(=O)N[C@@H](C)C(=O)Nc3nc(=O)n([C@@H]4O[C@H](CC)[C@@H](O)[C@H]4O)cc3F)C(C)C)NC(=O)CCCN)C(C)C)nc2=O)[C@H](O)[C@@H]1O]

Compound 538 (1.022 g, 0.582 mmol) in dioxane (10 m₁) on ice batch was added HCl (36%, 3 m₁). The mixture was stirred on ice bath for 30 min, diluted with dioxane (10 m₁) and toluene (10 m₁), concentrated at 2 −8° C. in vacuo, and co-evaporated two times with dioxane (10 m₁) and toluene (10 m₁) at 2 −8° C. in vacuo, and dried with oil vacuum pump to afford the title crude product (0.980 g, >100 yield) for direct use in the next step without further purification. MS-ESI (m/z): [M+H]⁺ calcd for: C₇H₁₀₉F₃N₁₇O₂₅, 1656.7733; found: 1656.7980 Example 372. Synthesis of (S)-2-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)-N5-((2S,5S)-1-((1-((2R,3R,4S,5R)-5-ethyl-3,4-dihydroxytetrahydrofuran-2-yl)-5-fluoro-2-oxo-1,2-dihydropyrimidin-4-yl)amino)-12,12-bis((3-(((S)-1-(((S)-1-((1-((2R,3R,4S,5R)-5-ethyl-3,4-dihydroxytetrahydrofuran-2-yl)-5-fluoro-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)pentanediamide, 540.

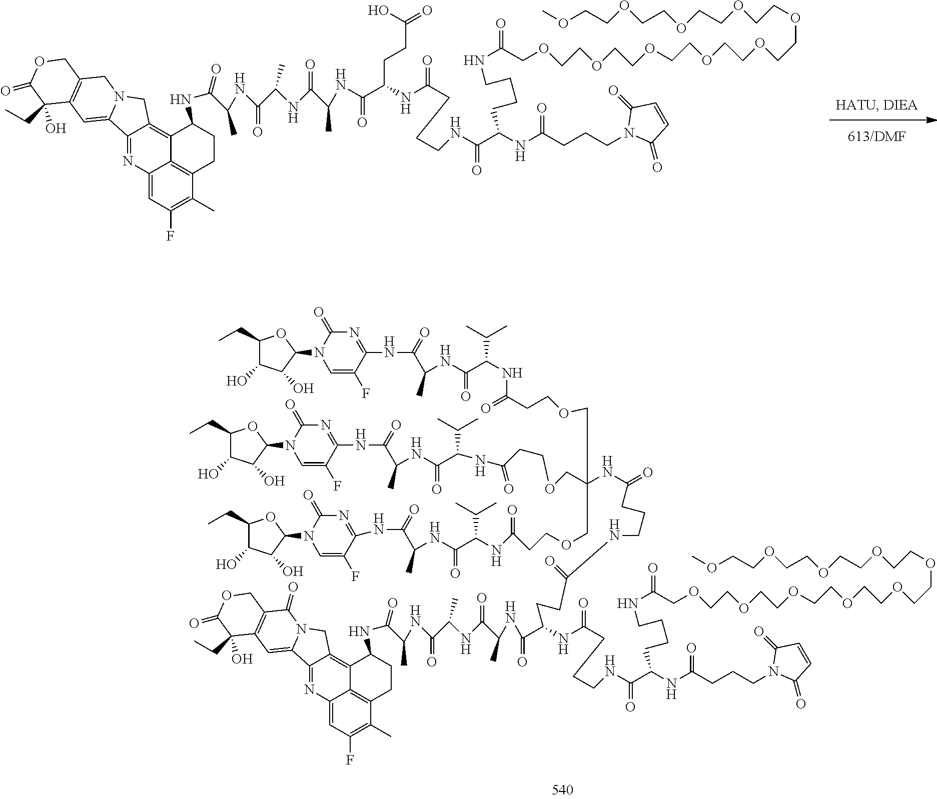

A solution of (37S,45S)-37-(4-(2,5-dioxo-2,5-dihydro-1H-pyrrol-1-yl)butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oic acid (250 mg, 0.154 mmol), compound 619 (261 mg, 0.154 mmol), HATU (195 mg, 0.512 mmol), DIPEA (0.050 m₁, 0.286 mmol) in DMF (6 m₁) was stirred for 8 h, evaporated and purified by C18 prep HPLC (25 mm×250 mm) with elution of MeOH/H₂O (1:9 to 3:3 in 50 min at 25 ml/min) to afford the title compound, 165 mg (65% yield). MS-ESI (m/z): [M+H]⁺ calcd for: C₁₄₈H₂₁₇F₄N₂₈O₅₀, 3262.5235; found: 32262.5488.

### Example 373. Synthesis of (S)—N¹—((S)-1-(((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)-propoxy)methyl)amino)-1-oxopropan-2-yl)amino)-3-methyl-1-oxobutan-2-yl)-N⁵-((2S,5S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-12,12-bis((3-(((S)-1-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)-2-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)pentanediamide, 541

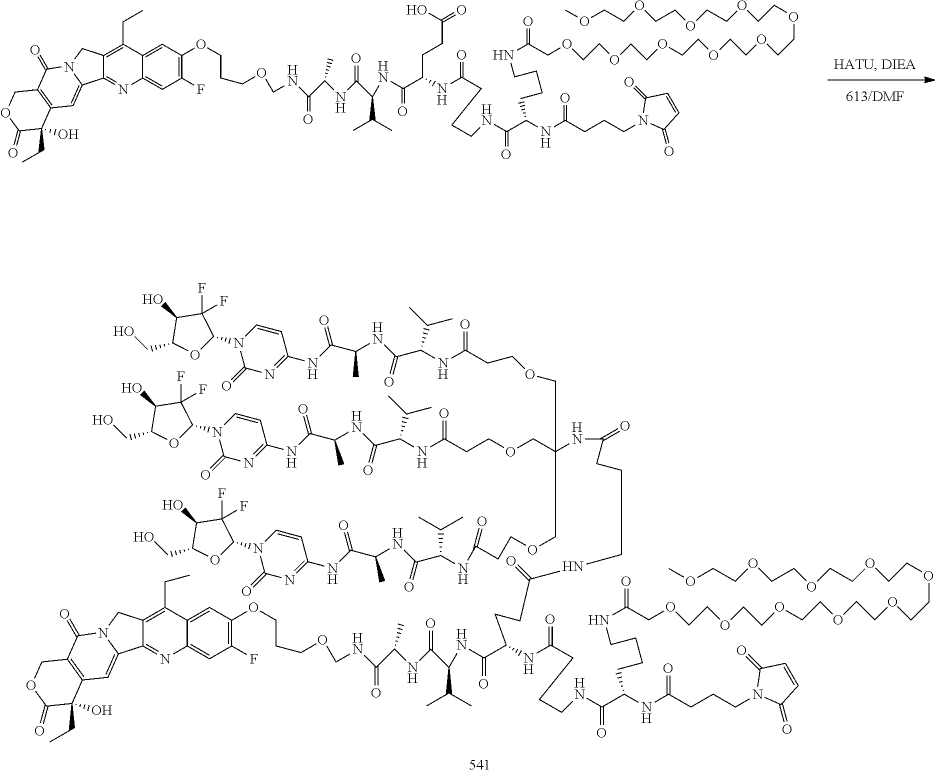

A solution of (37S,45S)-45-(((S)-1-(((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)-methyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazaoctatetracontan-48-oic acid (251 mg, 0.152 mmol), compound 613 (255 mg, 0.153 mmol), HATU (191 mg, 0.500 mmol), DIPEA (0.050 m₁, 0.286 mmol) in DMF (6 m₁) was stirred for 8 h, evaporated and purified by C18 prep HPLC (50 mm×250 mm) with elution of MeOH/H2O (1:9 to 3:1 in 50 min at 30 ml/min) to afford the title compound, 315 mg (63% yield). MS-ESI (m/z): [M+H]⁺ calcd for: C₁₄₆H₂₁₃F₇N₂₇O₅₁, 3293.4793; found: 3293.4855.

### Example 374. Synthesis of (S)-N5-(4-((1,3-bis(3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propan-2-yl)amino)-4-oxobutyl)-Ni—((S)-1-(((S)-1-(((3-(((S)-4,11-diethyl-8-fluoro-4-hydroxy-3,14-dioxo-3,4,12,14-tetrahydro-1H-pyrano[3′,4′:6,7]-indolizino[1,2-b]quinolin-9-yl)oxy)propoxy)methyl)amino)-1-oxopropan-2-yl)amino)-3-methyl-1-oxobutan-2-yl)-2-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)pentanediamide, 542

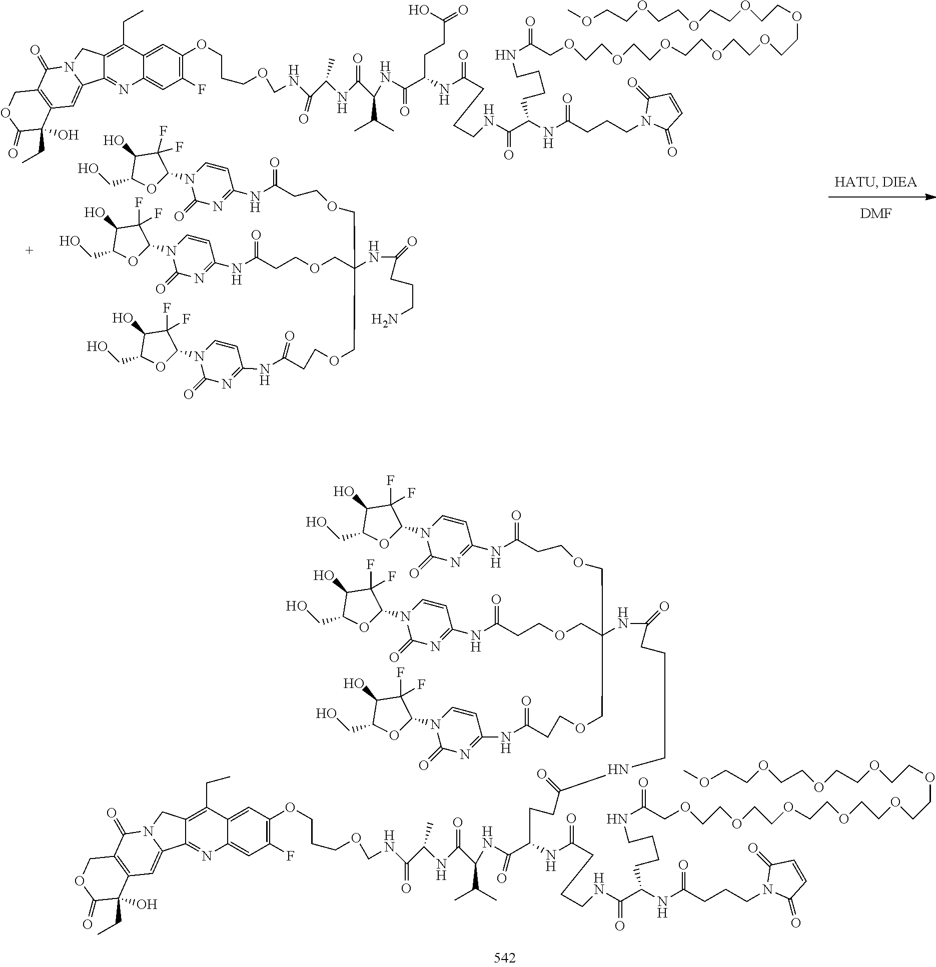

A solution of (37S,45S)-45-(((S)-1-(((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)-methyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazaoctatetracontan-48-oic acid (255 mg, 0.154 mmol), compound 613 (180 mg, 0.155 mmol), HATU (191 mg, 0.500 mmol), DIPEA (0.050 m₁, 0.286 mmol) in DMF (6 m₁) was stirred for 8 h, evaporated and purified by C18 prep HPLC (50 mm×250 mm) with elution of MeOH/H₂O (1:9 to 3:1 in 50 min at 30 ml/min) to afford the title compound, 278 mg (65% yield). MS-ESI (m/z): [M+H]⁺ calcd for: C₁₂₂H₁₇₁F₇N₂₁O₄₅, 2783.1627; found: 2783.1892.

### Example 375. Synthesis of((S)-2-((S)-6-((((9H-fluoren-9-yl)methoxy)carbonyl)amino)-2-(4-((3aR,4R,7S,7aS)-1,3-dioxo-1,3,3a,4,7,7a-hexahydro-2H-4,7-epoxyisoindol-2-yl)butanamido)hexanamido)-5-(tert-butoxy)-5-oxopentanoyl)-L-valine

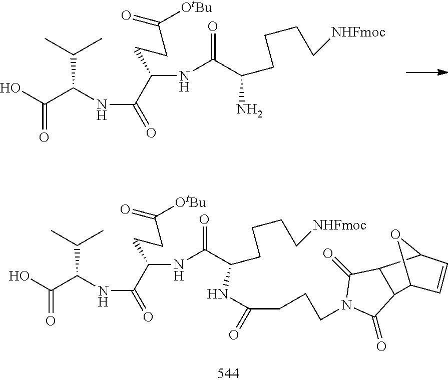

2,5-dioxopyrrolidin-1-yl 4-((3aR,4R,7S,7aS)-1,3-dioxo-1,3,3a,4,7,7a-hexahydro-2H-4,7-epoxyisoindol-2-yl)butanoate (1.862 g, 5.35 mmol) and ((S)-2-((S)-6-((((9H-fluoren-9-yl)methoxy)carbonyl)amino)-2-aminohexanamido)-5-(tert-butoxy)-5-oxopentanoyl)-L-valine (2.658 g, 4.074 mmol) in DMF (80 mL) was added DIPEA (222 mg, 0.30 m₁, 1.72 mmol) at 0° C. The mixture was then stirred at RT for 2h, concentrated and purified by SiO₂ column with elution of MeOH/DCM (1:10 to 1:5) containing 1% HOAc to give the title compound, 2.633 g (73% yield), as a white solid, MS-ESI (m/z): [M+H]⁺ calcd for: C₄₇H₆₀N₅O₁₂, 886.4239; found: 886.4485.

### Example 376. Synthesis of((S)-2-((S)-6-((((9H-fluoren-9-yl)methoxy)carbonyl)amino)-2-(4-((3aR,4R,7S,7aS)-1,3-dioxo-1,3,3a,4,7,7a-hexahydro-2H-4,7-epoxyisoindol-2-yl)butanamido) hexanamido)-5-(tert-butoxy)-5-oxopentanoyl)-L-alanine

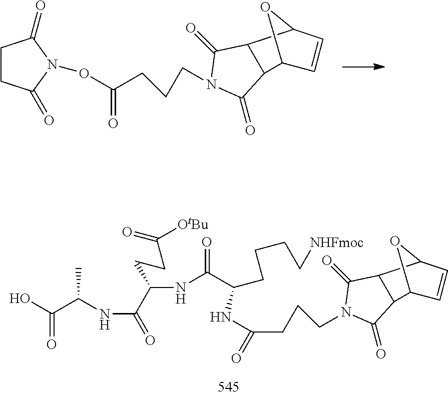

2,5-dioxopyrrolidin-1-yl 4-((3aR,4R,7S,7aS)-1,3-dioxo-1,3,3a,4,7,7a-hexahydro-2H-4,7-epoxyisoindol-2-yl)butanoate (883 mg, 2.39 mmol) and ((S)-2-((S)-6-((((9H-fluoren-9-yl)methoxy)carbonyl)amino)-2-aminohexanamido)-5-(tert-butoxy)-5-oxopentanoyl)-L-alanine (1.255 g, 2.01 mmol) in DMF (50 mL) was added DIPEA (0.22 g, 0.30 m₁, 1.72 mmol) at 0° C. The mixture was then stirred at RT for 2h, concentrated and purified by SiO₂ column with elution of MeOH/DCM (1:10 to 1:5) containing 1% HOAc to give the title, 1.171 g (68% yield), as a white solid, MS-ESI (m/z): [M+H]⁺ calcd for: C₄₅H₅₆N₅O₁₂, 858.3926; found: 858.4105.

### Example 377. Synthesis of ((S)-2-((S)-6-amino-2-(4-((3aR,4R,7S,7aS)-1,3-dioxo-1,3,3a,4,7,7a-hexahydro-2H-4,7-epoxyisoindol-2-yl)butanamido)hexanamido)-5-(tert-butoxy)-5-oxopentanoyl)-L-valine

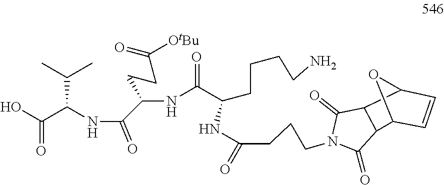 [SMILES: CC(C)[C@H](NC(=O)[C@H](CCC(=O)OC(C)(C)C)NC(=O)[C@H](CCCCN)NC(=O)CCCN1C(=O)C2C3C=CC(O3)C2C1=O)C(=O)O]

Compound 545 (1.746 g, 1.971 mmol) in DMF (20 m₁) on ice bath was added morpholine (10 m₁). The mixture was stirred for 30 min on ice bath, diluted with DMF (20 mL), evaporated and co-evaporated with DMF (10 m₁) and dried with oil pump to afford the title compound (1.312 g, >100% yield), which was used directly for the next step without further purification. MS-ESI (m/z): [M+H]⁺ calcd for: C₃₂H₅₀N₅O₁₀, 664.3558; found: 664.3787.

### Example 378. Synthesis of((S)-2-((S)-6-amino-2-(4-((3aR,4R,7S,7aS)-1,3-dioxo-1,3,3a,4,7,7a-hexahydro-2H-4,7-epoxyisoindol-2-yl)butanamido)hexanamido)-5-(tert-butoxy)-5-oxopentanoyl)-L-alanine

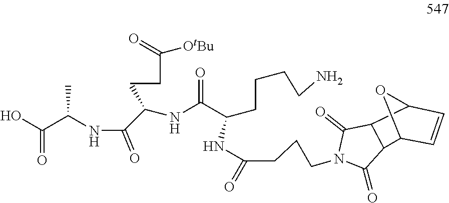 [SMILES: C[C@H](NC(=O)[C@H](CCC(=O)OC(C)(C)C)NC(=O)[C@H](CCCCN)NC(=O)CCCN1C(=O)C2C3C=CC(O3)C2C1=O)C(=O)O]

((S)-2-((S)-6-((((9H-fluoren-9-yl)methoxy)carbonyl)amino)-2-(4-((3aR,4R,7S,7aS)-1,3-dioxo-1,3,3a,4,7,7a-hexahydro-2H-4,7-epoxyisoindol-2-yl)butanamido)hexanamido)-5-(tert-butoxy)-5-oxopentanoyl)-L-alanine (1.358 g, 1.584 mmol) in DMF (20 m₁) on ice bath was added morpholine (10 m₁). The mixture was stirred for 30 min on ice bath, diluted with DMF (20 mL), evaporated and co-evaporated with DMF (10 m₁) and dried with oil pump to afford the title compound (1.011 g, >100% yield), which was used directly for the next step without further purification. MS-ESI (m/z): [M+H]⁺ calcd for: C₃₀H₄₆N₅O₁₀, 636.3245; found: 636.3408.

### Example 379. Synthesis of ((2S)-5-(tert-butoxy)-2-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)-5-oxopentanoyl)-L-valine, 548

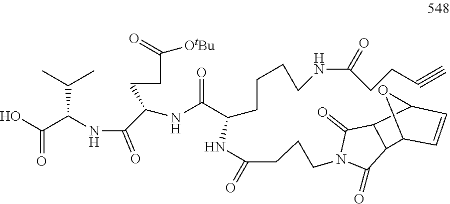 [SMILES: C#CCCC(=O)NCCCC[C@H](NC(=O)CCCN1C(=O)C2C3C=CC(O3)C2C1=O)C(=O)N[C@@H](CCC(=O)OC(C)(C)C)C(=O)N[C@H](C(=O)O)C(C)C]

Compound 546 (1.326 g, ~2.00 mmol) and compound 509 (495 mg, 2.50 mmol) in DMF (20 m₁) was added DIPEA (0.30 m₁, 1.72 mmol). The mixture was stirred for 2 h, evaporated and purified on SiO₂ column with elution of MeOH/DCM (1:4) containing 0.1% HOAc to afford the title compound, 936 mg (63% yield in two steps). MS-ESI (m/z): [M+H]⁺ calcd for: C₃₇H₅₄N₅O₁₁, 744.3821; found: 744.4005.

### Example 380. Synthesis of((2S)-5-(tert-butoxy)-2-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)-5-oxopentanoyl)-L-alanine

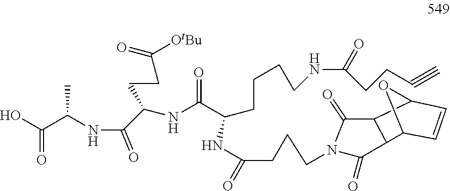 [SMILES: C#CCCC(=O)NCCCC[C@H](NC(=O)CCCN1C(=O)C2C3C=CC(O3)C2C1=O)C(=O)N[C@@H](CCC(=O)OC(C)(C)C)C(=O)N[C@@H](C)C(=O)O]

Compound 547 (1.00 g, ~1.61 mmol) and compound 509 (395 mg, 2.00 mmol) in DMF (20 m₁) was added DIPEA (0.40 m₁, 2.29 mmol). The mixture was stirred for 2 h, evaporated and purified on SiO₂ column with elution of MeOH/DCM (1:4) containing 0.1% HOAc to afford the title compound, 748 mg (65% yield in two steps). MS-ESI (m/z): [M+H]⁺ calcd for: C₃₅H₅₀N₅O₁₁, 716.3508; found: 716.3726.

### Example 381. Synthesis of tert-butyl (8S,11S,14S)-1-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)-14-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)-11-isopropyl-8-methyl-7,10,13-trioxo-4-oxa-6,9,12-triazaheptadecan-17-oate, 550

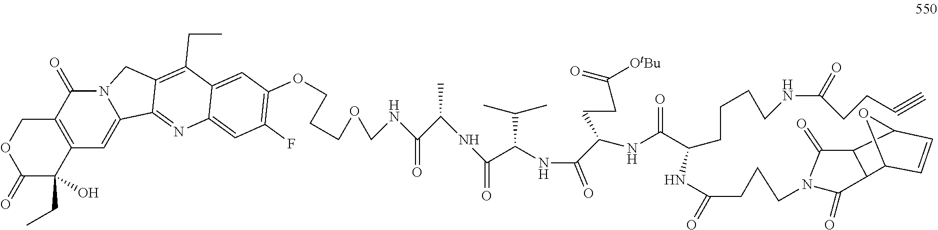 [SMILES: C#CCCC(=O)NCCCC[C@H](NC(=O)CCCN1C(=O)C2C3C=CC(O3)C2C1=O)C(=O)N[C@@H](CCC(=O)OC(C)(C)C)C(=O)N[C@H](C(=O)N[C@@H](C)C(=O)NCOCCCOc1cc2c(CC)c3c(nc2cc1F)-c1cc2c(c(=O)n1C3)COC(=O)[C@]2(O)CC)C(C)C]

To a mixture of compound 548 (452 mg, 0.620 mmol) and compound 514 (355 mg, 0.624 mmol) in DMA (10 m₁) was added EDC (401 mg, 2.077 mmol). The mixture was stirred for 6 h, evaporated and purified on SiO₂ column with elution of EtOAc/DCM (1:4) to afford the title compound, 539 mg (68% yield). MS-ESI (m/z): [M+H]⁺ calcd for: C₆₆H₈₅FN₉O₁₇, 1294.6048; found: 1294.6310.

### Example 382. Synthesis of (8S,11S,14S)-1-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)-14-((2S)-2-(4-((4R,7S)-1,3-dioxo-1,3,3a,4,7,7a-hexahydro-2H-4,7-epoxyisoindol-2-yl)butanamido)-6-(pent-4-ynamido)-hexanamido)-11-isopropyl-8-methyl-7,10,13-trioxo-4-oxa-6,9,12-triazaheptadecan-17-oic acid

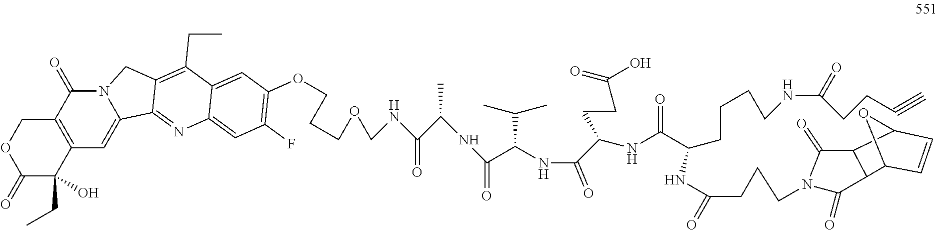 [SMILES: C#CCCC(=O)NCCCC[C@H](NC(=O)CCCN1C(=O)C2C3C=CC(O3)C2C1=O)C(=O)N[C@@H](CCC(=O)O)C(=O)N[C@H](C(=O)N[C@@H](C)C(=O)NCOCCCOc1cc2c(CC)c3c(nc2cc1F)-c1cc2c(c(=O)n1C3)COC(=O)[C@]2(O)CC)C(C)C]

Compound 550 (537 mg, 0.415 mmol) in dioxane (10 m₁) on ice batch was added HCl (36%, 3 m₁). The mixture was stirred on ice bath for 30 min, diluted with dioxane (10 m₁) and toluene (10 m₁), concentrated at 2 −8° C. in vacuo, and co-evaporated two times with dioxane (10 m₁) and toluene (10 m₁) at 2 ~8° C. in vacuo, and dried with oil vacuum pump to afford the title crude product (0.511 g, >100 yield) for direct use in the next step without further purification. MS-ESI (m/z): [M+H]⁺ calcd for: C₆₂H₇₇FN₉O₁₇, 1238.5422; found: 1238.5675.

### Example 383. Synthesis of(2S)—N⁵-(4-((1,3-bis(3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propan-2-yl)amino)-4-oxobutyl)-N′—((S)-1-(((S)-1-(((3-(((S)-4,11-diethyl-8-fluoro-4-hydroxy-3,14-dioxo-3,4,12,14-tetrahydro-1H-pyrano[3′,4′:6,7]-indolizino[1,2-b]quinolin-9-yl)oxy)propoxy)methyl)amino)-1-oxopropan-2-yl)amino)-3-methyl-1-oxobutan-2-yl)-2-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)pentanediamide

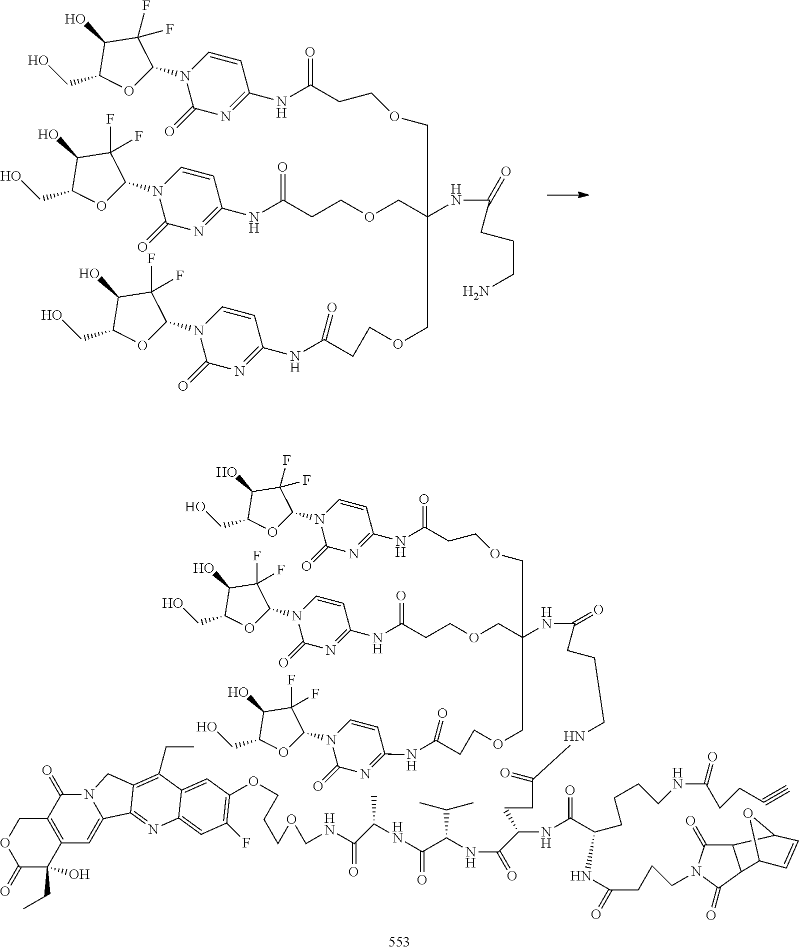

A solution of 3,3′-((2-(4-aminobutanamido)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propane-1,3-diyl)bis(oxy))bis(N-(1-((2R,4R,5R)-3,3-difluoro-4-hydroxy-5-(hydroxymethyl)tetrahydrofuran-2-yl)-2-oxo-1,2-dihydropyrimidin-4-yl)propanamide) (261 mg, 0.225 mmol), compound 551 (280 mg, 0.225 mmol), HATU (249 mg, 0.651 mmol), DIPEA (0.050 m₁, 0.286 mmol) in DMF (7 m₁) was stirred for 8 h, evaporated and purified by C₁₈ prep HPLC (50 mm×250 mm) with elution of MeOH/H₂O (1:9 to 3:1 in 50 min at 30 ml/min) to afford the title compound, 326 mg (61% yield). MS-ESI (m/z): [M+H]⁺ calcd for: C₁₀₆H₁₃₂F₇N₂₀O₃₅, 2377.9053; found: 2377.9298.

### Example 384. Synthesis of tert-butyl (4S)-4-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)-5-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)amino)-5-oxopentanoate

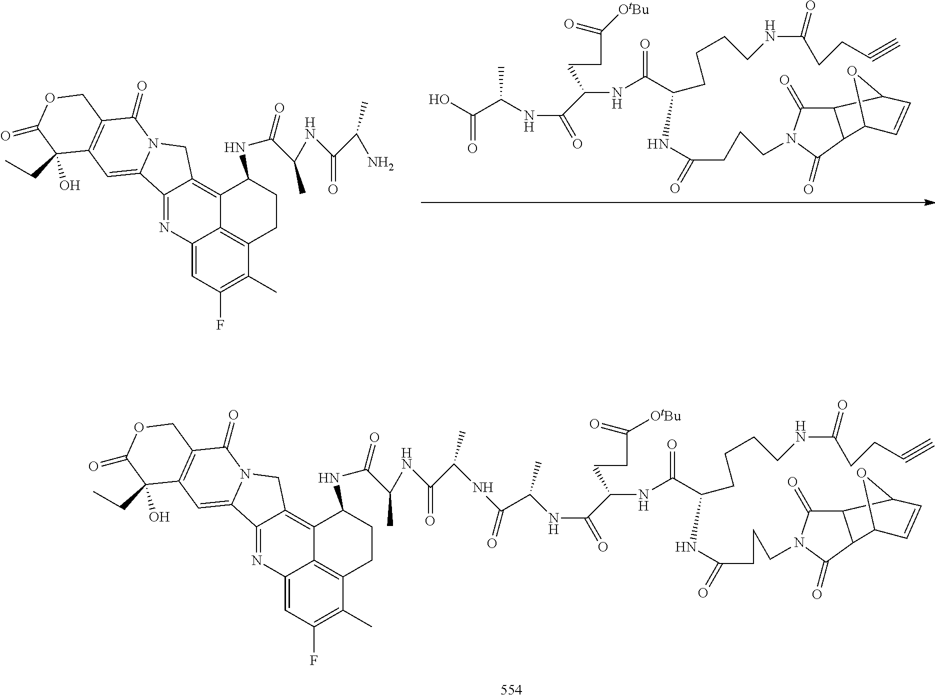 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](C)N)CC3 ; C#CCCC(=O)NCCCC[C@H](NC(=O)CCCN1C(=O)C2C3C=CC(O3)C2C1=O)C(=O)N[C@@H](CCC(=O)OC(C)(C)C)C(=O)N[C@@H](C)C(=O)O ; C#CCCC(=O)NCCCC[C@H](NC(=O)CCCN1C(=O)C2C3C=CC(O3)C2C1=O)C(=O)N[C@@H](CCC(=O)OC(C)(C)C)C(=O)N[C@@H](C)C(=O)N[C@@H](C)C(=O)N[C@@H](C)C(=O)N[C@H]1CCc2c(C)c(F)cc3nc4c(c1c23)Cn1c-4cc2c(c1=O)COC(=O)[C@]2(O)CC]

To a mixture of compound 549 (435 mg, 0.608 mmol) and compound 522, HCl salt (376 mg, 0.613 mmol) in DMA (10 m₁) was added EDC (400 mg, 2.076 mmol). The mixture was stirred for 8 h, evaporated and purified on SiO₂ column with elution of EtOAc/DCM (1:4) to afford the title compound, 539 mg (68% yield). MS-ESI (m/z): [M+H]⁺ calcd for: C₆₅H₈₀FN₁₀O₁₆, 1275.5739; found: 1275.5975.

### Example 385. Synthesis of(4S)-4-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)-5-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)amino)-5-oxopentanoic acid

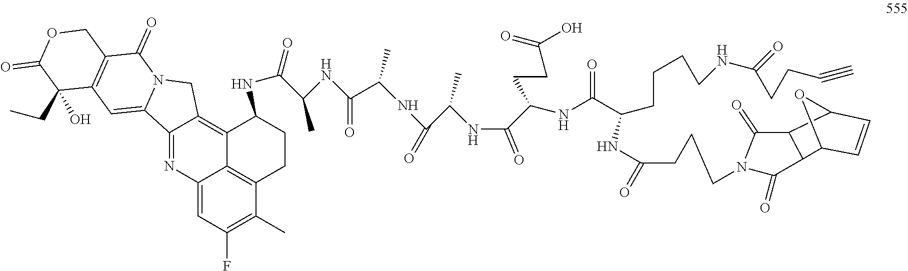 [SMILES: C#CCCC(=O)NCCCC[C@H](NC(=O)CCCN1C(=O)C2C3C=CC(O3)C2C1=O)C(=O)N[C@@H](CCC(=O)O)C(=O)N[C@@H](C)C(=O)N[C@@H](C)C(=O)N[C@@H](C)C(=O)N[C@H]1CCc2c(C)c(F)cc3nc4c(c1c23)Cn1c-4cc2c(c1=O)COC(=O)[C@]2(O)CC]

Compound 554 (426 mg, 0.334 mmol) in dioxane (10 m₁) on ice batch was added HCl (36%, 3 m₁). The mixture was stirred on ice bath for 30 min, diluted with dioxane (10 m₁) and toluene (10 m₁), concentrated at 2 −8° C. in vacuo, and co-evaporated two times with dioxane (10 m₁) and toluene (10 m₁) at 2 −8° C. in vacuo, and dried with oil vacuum pump to afford the title crude product (410 mg, >100% yield) for direct use in the next step without further purification. MS-ESI (m/z): [M+H]⁺ calcd for: C₆₁H₇₂FN₁₀O₁₆, 1219.5113; found: 1219.5385.

### Example 386. Synthesis of (2S)-N5-(4-((1,3-bis(3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propan-2-yl)amino)-4-oxobutyl)-2-((2S)-2-(4-((4R,7S)-1,3-dioxo-1,3,3a,4,7,7a-hexahydro-2H-4,7-epoxyisoindol-2-yl)butanamido)-6-(pent-4-ynamido)hexanamido)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-1-oxopropan-2-yl)pentanediamide

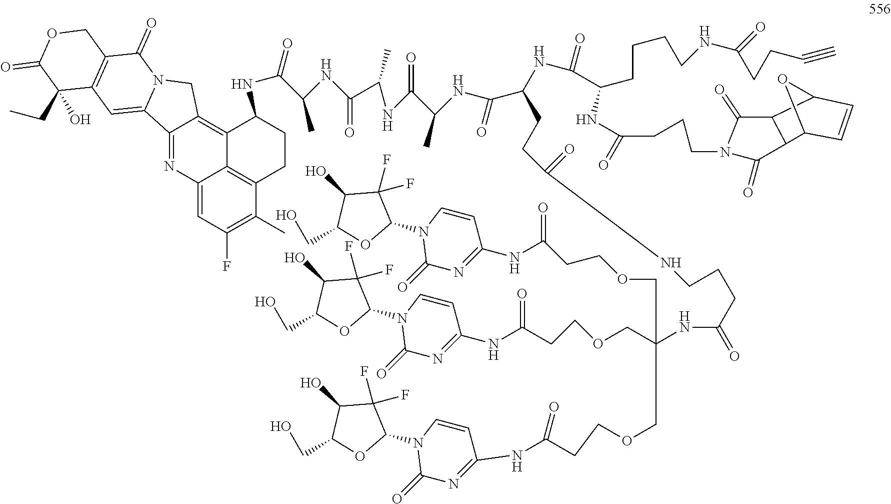 [SMILES: C#CCCC(=O)NCCCC[C@H](NC(=O)CCCN1C(=O)C2C3C=CC(O3)C2C1=O)C(=O)N[C@@H](CCC(=O)NCCCC(=O)NC(COCCC(=O)Nc1ccn([C@@H]2O[C@H](CO)[C@@H](O)C2(F)F)c(=O)n1)(COCCC(=O)Nc1ccn([C@@H]2O[C@H](CO)[C@@H](O)C2(F)F)c(=O)n1)COCCC(=O)Nc1ccn([C@@H]2O[C@H](CO)[C@@H](O)C2(F)F)c(=O)n1)C(=O)N[C@@H](C)C(=O)N[C@@H](C)C(=O)N[C@@H](C)C(=O)N[C@H]1CCc2c(C)c(F)cc3nc4c(c1c23)Cn1c-4cc2c(c1=O)COC(=O)[C@]2(O)CC]

A solution of 3,3′-((2-(4-aminobutanamido)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-methyl)propane-1,3-diyl)bis(oxy))bis(N-(1-((2R,4R,5R)-3,3-difluoro-4-hydroxy-5-(hydroxymethyl)tetrahydrofuran-2-yl)-2-oxo-1,2-dihydropyrimidin-4-yl)propanamide) (260 mg, 0.225 mmol), compound 555 (275 mg, 0.225 mmol), HATU (251 mg, 0.653 mmol), DIPEA (0.050 m₁, 0.286 mmol) in DMF (8 m₁) was stirred for 8 h, evaporated and purified by C₁₈ prep HPLC (50 mm×250 mm) with elution of MeOH/H₂O (1:9 to 3:1 in 50 min at 30 ml/min) to afford the title compound, 302 mg (57% yield). MS-ESI (m/z): [M+H]⁺ calcd for: C₁₀₅H₁₂₇F₇N₂₁O₃₄, 2358.8743; found: 2358.9005.

### Example 387. Synthesis of tert-butyl (8S,11S,14S)-14-((S)-2-((((9H-fluoren-9-yl)methoxy)carbonyl)amino)-6-(pent-4-ynamido)hexanamido)-1-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)-11-isopropyl-8-methyl-7,10,13-trioxo-4-oxa-6,9,12-triazaheptadecan-17-oate, 558

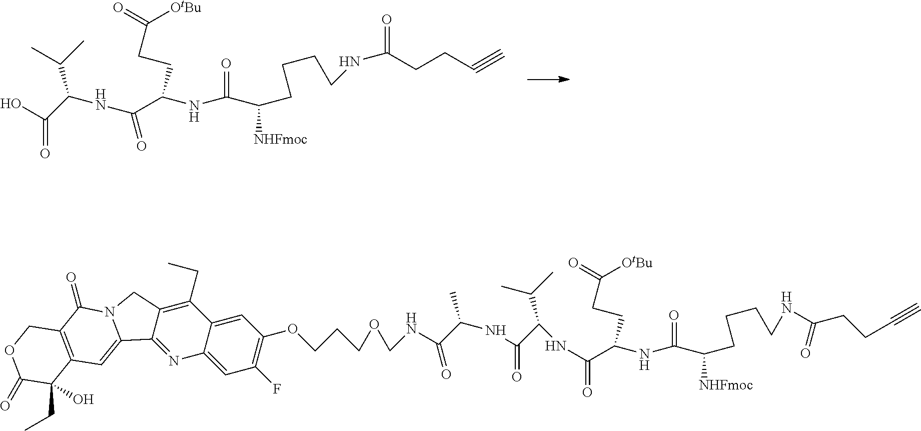

((S)-2-((S)-2-((((9H-fluoren-9-yl)methoxy)carbonyl)amino)-6-(pent-4-ynamido)hexanamido)-5-(tert-butoxy)-5-oxopentanoyl)-L-valine (2.521 g, 3.440 mmol), compound 514 (1.961, 3.450 mmol) in DMA (40 m₁) was added EDC (2.25 g, 11.65 mmol). The mixture was stirred for 8 h, evaporated and purified on SiO₂ column with elution of EtOAc/DCM (1:4) to afford the title compound, 2.603 g (59% yield). MS-ESI (m/z): [M+H]⁺ calcd for: C₆₉H₈₄FN₈O₁₅, 1283.6041; found: 1283.6383.

### Example 388. Synthesis of (8S,11S,14S)-14-((S)-2-((((9H-fluoren-9-yl)methoxy)carbonyl)amino)-6-(pent-4-ynamido)hexanamido)-1-(((S)-4,11-diethyl-8-fluoro-4-hydroxy-3,14-dioxo-3,4,12,14 tetrahydro-1H-pyrano[3′,4′:6,7]indolizino[1,2-b]quinolin-9-yl)oxy)-11-isopropyl-8-methyl-7,10,13-trioxo-4-oxa-6,9,12-triazaheptadecan-17-oic acid, 559

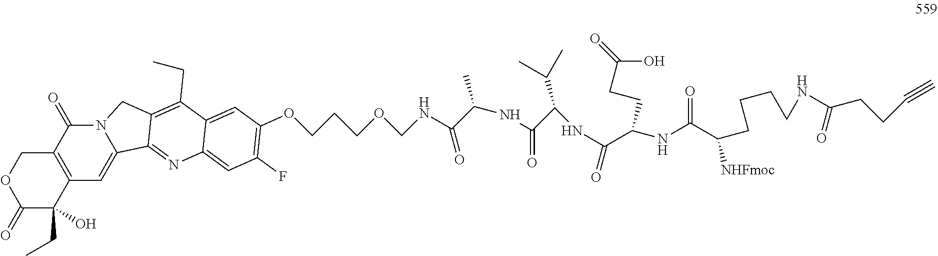

Compound 558 (1.321 g, 1.030 mmol) in dioxane (15 ml) on ice batch was added HCl (36%, 4 ml). The mixture was stirred on ice bath for 30 min, diluted with dioxane (20 ml) and toluene (20 ml), concentrated at 2 −8° C. in vacuo, and co-evaporated two times with dioxane (20 ml) and toluene (20 ml) at 2 −8° C. in vacuo, and dried with oil vacuum pump to afford the title crude product (1.265 g, >100% yield) for direct use in the next step without further purification. MS-ESI (m/z): [M+H]⁺ calcd for: C₆₅H₇₆FN₇O₁₅, 1227.5415; found: 1227.5765.

### Example 389. Synthesis of (9H-fluoren-9-yl)methyl ((19S,22S)-19-(((S)-1-(((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)methyl)amino)-1-oxopropan-2-yl)amino)-3-methyl-I-oxobutan-2-yl)carbamoyl)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-9,9-bis((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)-1,4,11,16,21,28-hexaoxo-7-oxa-3,10,15,20,27-pentaazadotriacont-31-yn-22-yl)carbamate, 560

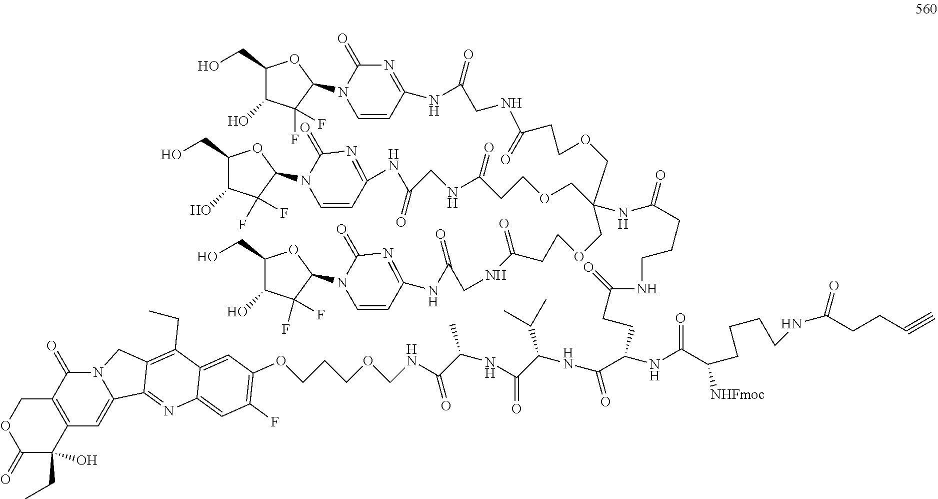

A solution of 3,3′-((2-(4-aminobutanamido)-2-((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)propane-1,3-diyl)bis(oxy))bis(N-(2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)-propanamide) (292 mg, 0.220 mmol), compound 559 (270 mg, 0.220 mmol), HATU (251 mg, 0.653 mmol), DIPEA (O_{.050} ml, 0.286 mmol) in DMF (8 ml) was stirred for 8 h, evaporated and purified by C₁₈ prep HPLC (50 mm×250 mm) with elution of MeOH/H₂O (1:9 to 3:1 in 50 min at 30 ml/min) to afford the title compound, 295 mg (53% yield). MS-ESI (m/z): [M+H]⁺ calcd for: C₁₁₅H₁₄₀F₇N₂₂O₃₆, 2537.9690; found: 2538.0055.

### Example 390. Synthesis of (S)-2-((S)-2-amino-6-(pent-4-ynamido)hexanamido)-N5-(4-((1,17-bis((1-((2R,4R,5R)-3,3-difluoro-4-hydroxy-5-(hydroxymethyl)tetrahydrofuran-2-yl)-2-oxo-1,2-dihydropyrimidin-4-yl)amino)-9-((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)-1,4,14,17-tetraoxo-7,11-dioxa-3,15-diazaheptadecan-9-yl)amino)-4-oxobutyl)-N1-((S)-I-(((S)-1-(((3-(((S)-4,11-diethyl-8-fluoro-4-hydroxy-3,14-dioxo-3,4,12,14-tetrahydro-1H-pyrano[3′,4′:6,7]indolizino[1,2-b]quinolin-9-yl)oxy)propoxy)methyl)amino)-1-oxopropan-2-yl)amino)-3-methyl-1-oxobutan-2-yl)pentanediamide, 561

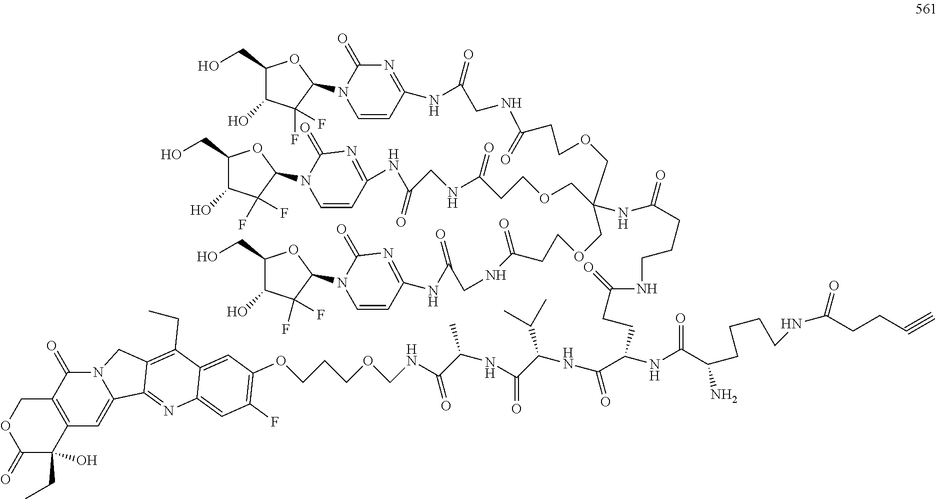 [SMILES: C#CCCC(=O)NCCCC[C@H](N)C(=O)N[C@@H](CCC(=O)NCCCC(=O)NC(COCCC(=O)NCC(=O)Nc1ccn([C@@H]2O[C@H](CO)[C@@H](O)C2(F)F)c(=O)n1)(COCCC(=O)NCC(=O)Nc1ccn([C@@H]2O[C@H](CO)[C@@H](O)C2(F)F)c(=O)n1)COCCC(=O)NCC(=O)Nc1ccn([C@@H]2O[C@H](CO)[C@@H](O)C2(F)F)c(=O)n1)C(=O)N[C@H](C(=O)N[C@@H](C)C(=O)NCOCCCOc1cc2c(CC)c3c(nc2cc1F)-c1cc2c(c(=O)n1C3)COC(=O)[C@]2(O)CC)C(C)C]

Compound 560 (285 mg, 0.112 mmol) in DMF (8 ml) on ice bath was added morpholine (8 ml). The mixture was stirred for 30 min on ice bath, diluted with DMF (10 mL), evaporated and co-evaporated with DMF (10 ml) and dried with oil pump to afford the title compound (266 mg, >100% yield), which was used directly for the next step without further purification. MS-ESI (m/z): [M+H]calcd for: C₁₀₀H₁₃₀F₇N₂₂O₂₄, 2315.9009; found: 2315.9450.

### Example 391. Synthesis of (2S,2'S)-2,2′-(((2S,19S)-10-((3aR,4R,7S,7aS)-1,3-dioxo-1,3,3a,4,7,7a-hexahydro-2H-4,7-epoxyisoindol-2-yl)-11-((3aR,4S,7R,7aS)-1,3-dioxo-1,3,3a,4,7,7a-hexahydro-2H-4,7-epoxyisoindol-2-yl)-4,9,12,17-tetraoxo-2,19-bis(4-(pent-4-ynamido)butyl)-3,8,13,18-tetraazaicosanedioyl)bis(azanediyl))bis(N5-(4-((1,17-bis((1-((2R,4R,5R)-3,3-difluoro-4-hydroxy-5-(hydroxymethyl)tetrahydrofuran-2-yl)-2-oxo-1,2-dihydropyrimidin-4-yl)amino)-9-((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)-1,4,14,17-tetraoxo-7,11-dioxa-3,15-diazaheptadecan-9-yl)amino)-4-oxobutyl)-N1-((S)-I-(((S)-1-(((3-(((S)-4,11-diethyl-8-fluoro-4-hydroxy-3,14-dioxo-3,4,12,14-tetrahydro-IH-pyrano[3′,4′:6,7]indolizino[1,2-b]quinolin-9-yl)oxy)propoxy)methyl)amino)-1-oxopropan-2-yl)amino)-3-methyl-I-oxobutan-2-yl)pentanediamide)

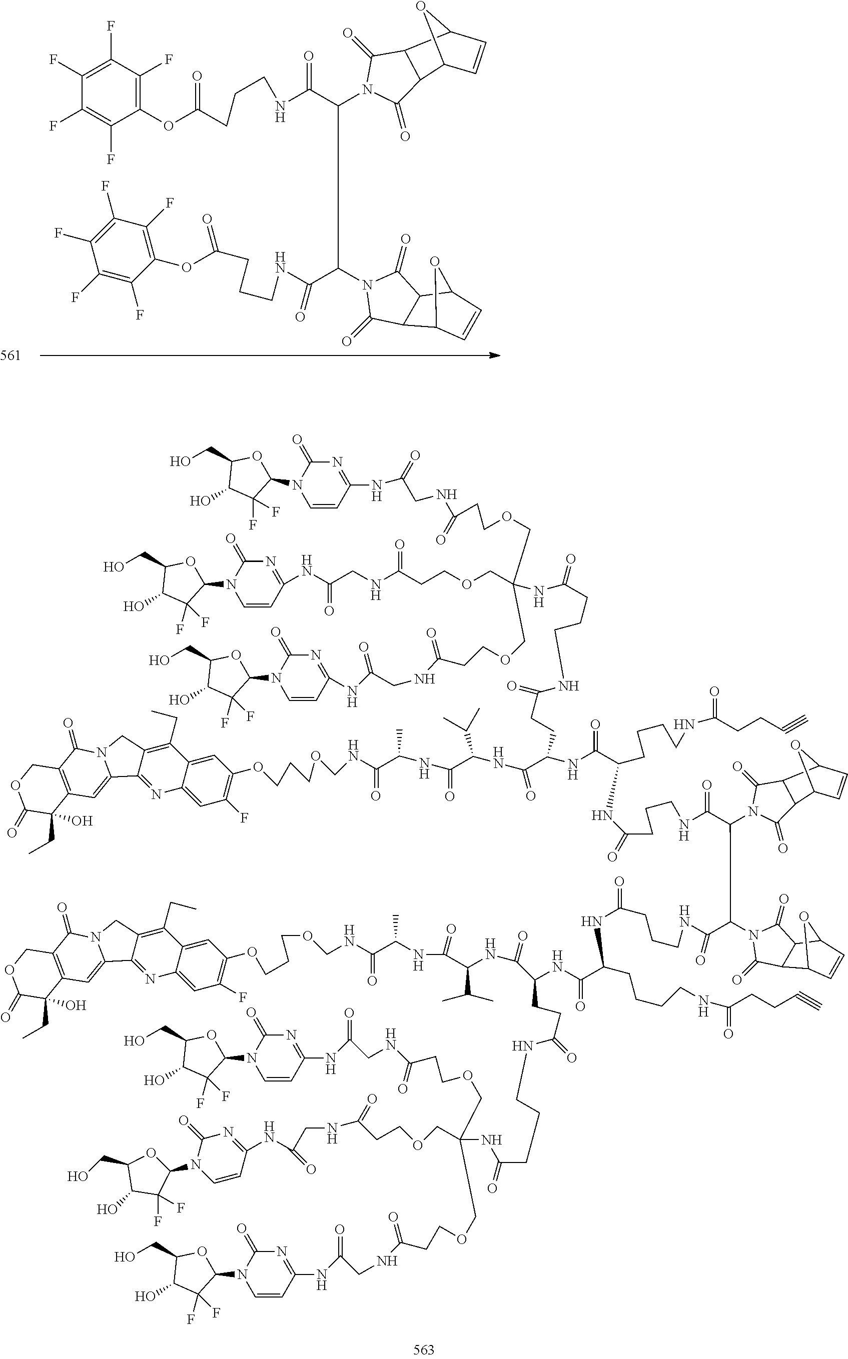

Compound 561 (260 mg, 0.112 mmol) and bis(perfluorophenyl) 4,4′-((2-((3aR,4R,7S,7aS)-1,3-dioxo-1,-3,3a,4,7,7a-hexahydro-2H-4,7-epoxyisoindol-2-yl)-3-((3aR,4S,7R,7aS)-1,3-dioxo-1,3,3a,4,7,7a-hexahydro-2H-4,7-epoxyisoindol-2-yl)succinyl)bis(azanediyl))dibutyrate (50 mg, 0.0526 mmol) in DMF (10 mL) was added DIPEA (89 mg, 0.05 ml, 0.286 mmol) at 0° C. The mixture was then stirred for 2 h at RT, concentrated and purified by Prep-HPLC with elution of water/CH₃H (20 CH₃OH to 80% CH₃OH in 45 min, C18 20 ×250 mm, 25 ml/mn) to give compound 563, 117 mg (43% yield), as a solid after lyophilization, MS-ESI (m/z): [M+H]⁺ calcd for: C₂₂₈H₂₈₅F₁₄N₄₈O₇₈, 5208.9587; found:5209.0225 (measured +calcd).

### Example 392. Synthesis of tert-butyl (S)-4-((S)-2-((((9H-fluoren-9-yl)methoxy)carbonyl)amino)-6-(pent-4-ynamido)hexanamido)-5-(((S)-1-(((S)-1-(((S)-1-(((1 S,9S)-9-ethyl-5-fluoro-9-hydroxy-4-methyl-10,13-dioxo-2,3,9,10,13,15-hexahydro-I H,12H-benzo[de]pyrano[3′,4′:6, 7]indolizino[1,2-b]quinolin-1l-yl)amino)-1-oxopropan-2-yl)amino)-1-oxopropan-2-yl)amino)-3-methyl-1-oxobutan-2-yl)amino)-5-oxopentanoate, 564

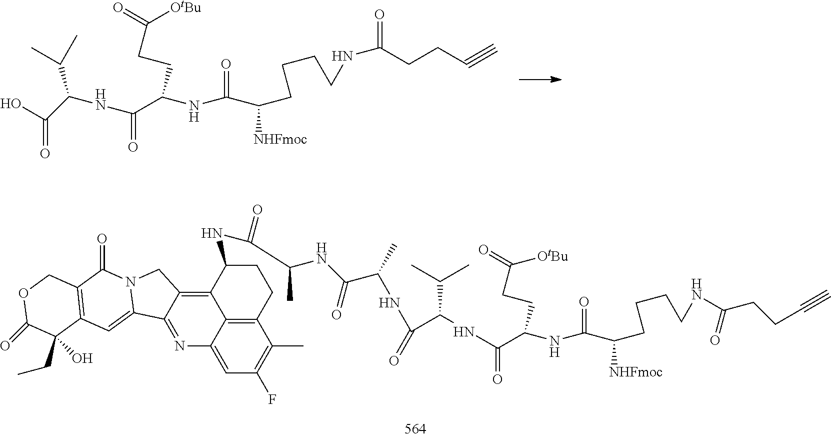

((S)-2-((S)-2-((((9H-fluoren-9-yl)methoxy)carbonyl)amino)-6-(pent-4-ynamido)hexanamido)-5-(tert-butoxy)-5-oxopentanoyl)-L-valine (2.123 g, 2.900 mmol), compound 522 (1.785, 2.901 mmol) in DMA (40 ml) was added EDC (2.25 g, 11.65 mmol). The mixture was stirred for 8 h, evaporated and purified on S₁₀₂ column with elution of EtOAc/DCM (1:4) to afford the title compound, 2.323 g (62% yield). MS-ESI (m/z): [M+H]⁺ calcd for: C₇₀H₈₃FN₉O₁₄, 1292.6044; found: 1292.6386.

### Example 393. Synthesis of (S)-4-((S)-2-((((9H-fluoren-9-yl)methoxy)carbonyl)amino)-6-(pent-4-ynamido)hexanamido)-5-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-3-methyl-1-oxobutan-2-yl)amino)-5-oxopentanoic acid, 565

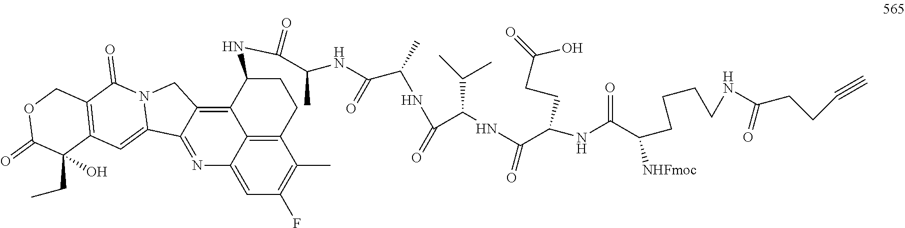

Compound 564 (1.125 g, 0.871 mmol) in dioxane (15 ml) on ice batch was added HCl (36%, 4 ml). The mixture was stirred on ice bath for 30 min, diluted with dioxane (20 ml) and toluene (20 ml), concentrated at 2 −8° C. in vacuo, and co-evaporated two times with dioxane (20 ml) and toluene (20 ml) at 2~8° C. in vacuo, and dried with oil vacuum pump to afford the title crude product (1.081 g, >100% yield) for direct use in the next step without further purification. MS-ESI (m/z): [M+H]⁺ calcd for: C₆₆H₇₅FN₉O₁₄, 1236.5418; found: 1236.5792.

### Example 394. Synthesis of (9H-fluoren-9-yl)methyl ((19S,22S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-9,9-bis((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)-19-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-3-methyl-I-oxobutan-2-yl)carbamoyl)-1,4,11,16,21,28-hexaoxo-7-oxa-3,10,15,20,27-pentaazadotriacont-31-yn-22-yl)carbamate, 566

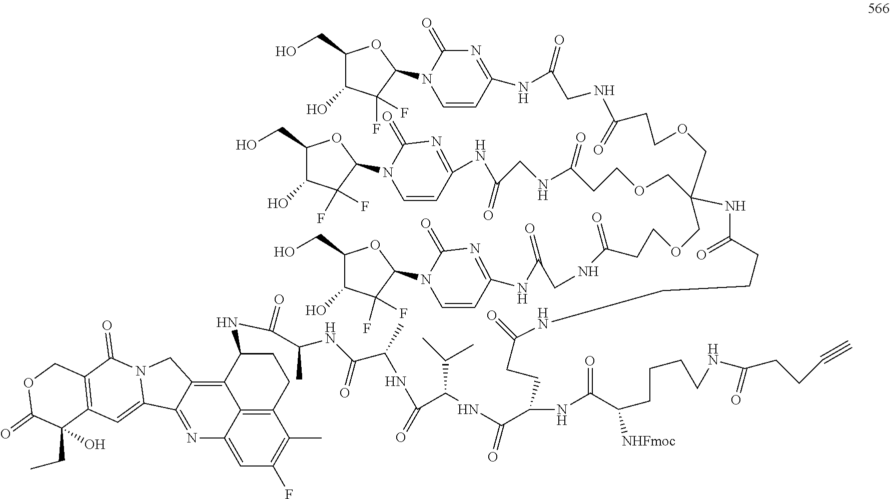

A solution of 3,3′-((2-(4-aminobutanamido)-2-((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)propane-1,3-diyl)bis(oxy))bis(N-(2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)-propanamide) (268 mg, 0.201 mmol), compound 565 (250 mg, 0.201 mmol), HATU (250 mg, 0.652 mmol), DIPEA (O_{.050} ml, 0.286 mmol) in DMF (8 ml) was stirred for 8 h, evaporated and purified by C₁₈ prep HPLC (50 mm×250 mm) with elution of MeOH/H₂O (1:9 to 3:1 in 50 min at 30 ml/min) to afford the title compound, 281 mg (55% yield). MS-ESI (m/z): [M+H]⁺ calcd for: C₁₁₆H₁₃₉F₇N₂₂O₃₅, 2546.9693; found: 2547.0085.

### Example 395. Synthesis of (S)-2-((S)-2-amino-6-(pent-4-ynamido)hexanamido)-N5-(4-((1,17-bis((1-((2R,4R,5R)-3,3-difluoro-4-hydroxy-5-(hydroxymethyl)tetrahydrofuran-2-yl)-2-oxo-1,2-dihydropyrimidin-4-yl)amino)-9-((3-((2-((1-((2R,4R,5R)-3,3-difluoro-4-hydroxy-5-(hydroxymethyl)-tetrahydrofuran-2-yl)-2-oxo-1,2-dihydropyrimidin-4-yl)amino)-2-oxoethyl)amino)-3-oxopropoxy)-methyl)-1,4,14,17-tetraoxo-7,11-dioxa-3,15-diazaheptadecan-9-yl)amino)-4-oxobutyl)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-3-methyl-1-oxobutan-2-yl)pentanediamide, 567

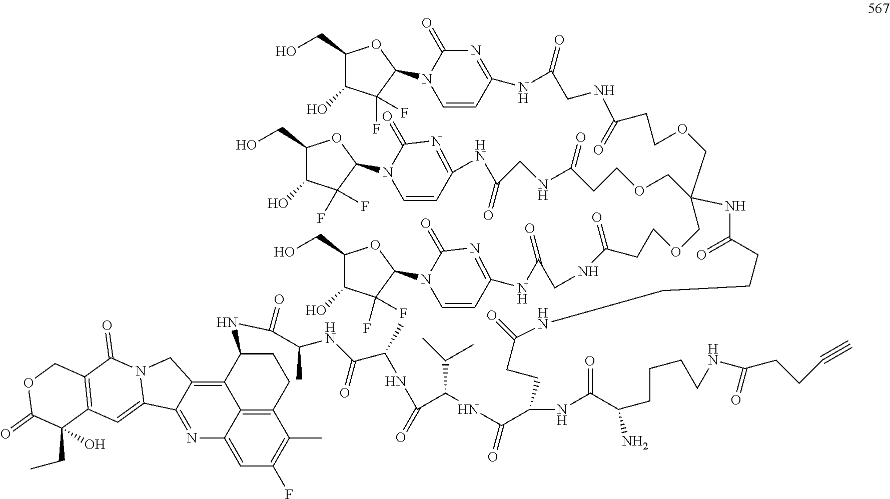 [SMILES: C#CCCC(=O)NCCCC[C@H](N)C(=O)N[C@@H](CCC(=O)NCCCC(=O)NC(COCCC(=O)NCC(=O)Nc1ccn([C@@H]2O[C@H](CO)[C@@H](O)C2(F)F)c(=O)n1)(COCCC(=O)NCC(=O)Nc1ccn([C@@H]2O[C@H](CO)[C@@H](O)C2(F)F)c(=O)n1)COCCC(=O)NCC(=O)Nc1ccn([C@@H]2O[C@H](CO)[C@@H](O)C2(F)F)c(=O)n1)C(=O)N[C@H](C(=O)N[C@@H](C)C(=O)N[C@@H](C)C(=O)N[C@H]1CCc2c(C)c(F)cc3nc4c(c1c23)Cn1c-4cc2c(c1=O)COC(=O)[C@]2(O)CC)C(C)C]

Compound 566 (275 mg, 0.108 mmol) in DMF (8 ml) on ice bath was added morpholine (8 ml). The mixture was stirred for 30 min on ice bath, diluted with DMF (10 mL), evaporated and co-evaporated with DMF (10 ml) and dried with oil pump to afford the title compound (266 mg, >100% yield), which was used directly for the next step without further purification. MS-ESI (m/z): [M+H]calcd for: C₁₀₁H₁₂₉F₇N₂₃O₃₃, 2324.9012; found: 2324.9425.

### Example 396. Synthesis of (2S,2'S)-2,2′-(((2S,19S)-10-((3aR,4R,7S,7aS)-1,3-dioxo-1,3,3a,4,7,7a-hexahydro-2H-4,7-epoxyisoindol-2-yl)-11-((3aR,4S,7R,7aS)-1,3-dioxo-1,3,3a,4,7,7a-hexahydro-2H-4,7-epoxyisoindol-2-yl)-4,9,12,17-tetraoxo-2,19-bis(4-(pent-4-ynamido)butyl)-3,8,13,18-tetraazaicosanedioyl)bis(azanediyl))bis(N5-(4-((1,17-bis((1-((2R,4R,5R)-3,3-difluoro-4-hydroxy-5-(hydroxymethyl)tetrahydrofuran-2-yl)-2-oxo-1,2-dihydropyrimidin-4-yl)amino)-9-((3-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-3-oxopropoxy)methyl)-1,4,14,17-tetraoxo-7,11-dioxa-3,15-diazaheptadecan-9-yl)amino)-4-oxobutyl)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-1-oxopropan-2-yl)amino)-3-methyl-1-oxobutan-2-yl)pentanediamide), 568

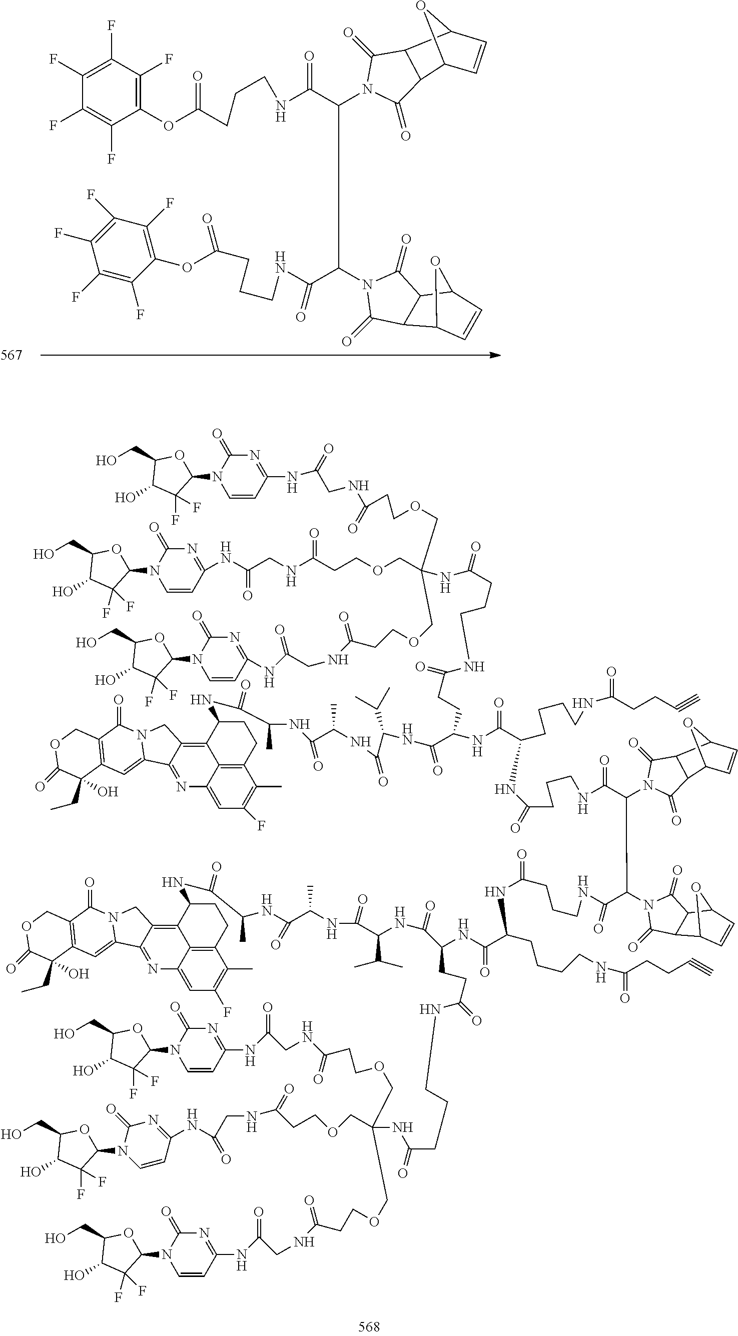

Compound 567 (255 mg, 0.110 mmol) and bis(perfluorophenyl) 4,4′-((2-((3aR,4R,7S,7aS)-1,3-dioxo-1, 3,3a,4, 7,7a-hexahydro-2H-4,7-epoxyisoindol-2-yl)-3-((3aR,4S,7R,7aS)-1,3-dioxo-1,3,3a,4,7,7a-hexahydro-2H-4,7-epoxyisoindol-2-yl)succinyl)bis(azanediyl))dibutyrate (50 mg, 0.0526 mmol) in DMF (10 mL) was added DIPEA (89 mg, 0.05 ml, 0.286 mmol) at 0° C. The mixture was then stirred for 2 h at RT, concentrated and purified by Prep-HPLC with elution of water/CH₃OH (20% CH₃OH to 80% CH₃OH in 45 min, C18 20 ×250 mm, 25 ml/min) to give compound 568, 107 mg (39% yield), as a solid after lyophilization, MS-ESI (m/z): [M+H]⁺ calcd for: C₂₃₀H₂₈₃F₁₄N₅₀O₇₆, 5226.9594; found:5226.9987.

### Example 397. General synthesis of CPT/linker complexes containing mono ligand compound via a click chemistry

In a glass tube with a screw cap, compound 529 (30 mg, 0.0254 mmol, 1.0 eq) or 531 (30 mg, 0.0275 mmol, 1.08 eq), or 551 (35 mg, 0.0282 mmol, 1.11 eq), or 553 (65 mg, 0.0273 mmol, 1.08 eq), or 555 (34 mg, 0.0279 mmol, 1.10 eq), or 556 (65 mg, 0.0275 mmol, 1.08 eq), and a ligand (compound 470~498) containing azido terminal (1.0~1.11 eq) dissolved in DMA (5 ml) was added sodium ascorbate (250 mM in H₂O, 0.510~0.560 ml, 0.128-0.140 mmol, 5.0-5.5 eq). After vortexed for a few seconds, a solution containing CuSO₄ and BTTPS (3-[4-({bis[(1-tert-butyl-1H-1,2,3-triazol-4-yl)methyl]amino}methyl)-1H-1,2,3-triazol-1-yl]propyl hydrogen sulphate) (DMA-H₂O 1:10, 10 mM CuSO₄, 11 mM BTTPS, 2.550~2.800 ml, 1.0-1.1 eq CuSO₄, 1.1-1.2 eq BTTPS) was added in, and the reaction mixture was vortexed and agitated on an orbital shaker at 25° C. for 18 h. After that, EDTA (200 mM, 0.450 ml, 0.900 mmol) was added, the mixture was vortexed, heated over hot water bath (80° C.) without the screw cap for 3 h for removal of the protected furan, concentrated in a rotary evaporator connected with a vacuum pump, and then purified by C18 prep-HPLC (20 mm×250 mm), eluted with water/CH₃CN from 25% CH₃CN to 90% CH₃CN in 50 min, 8 m₁/min. The fractions containing the products were pooled, evaporated, and lyophilized to afford the planned product (35%~83% yield).

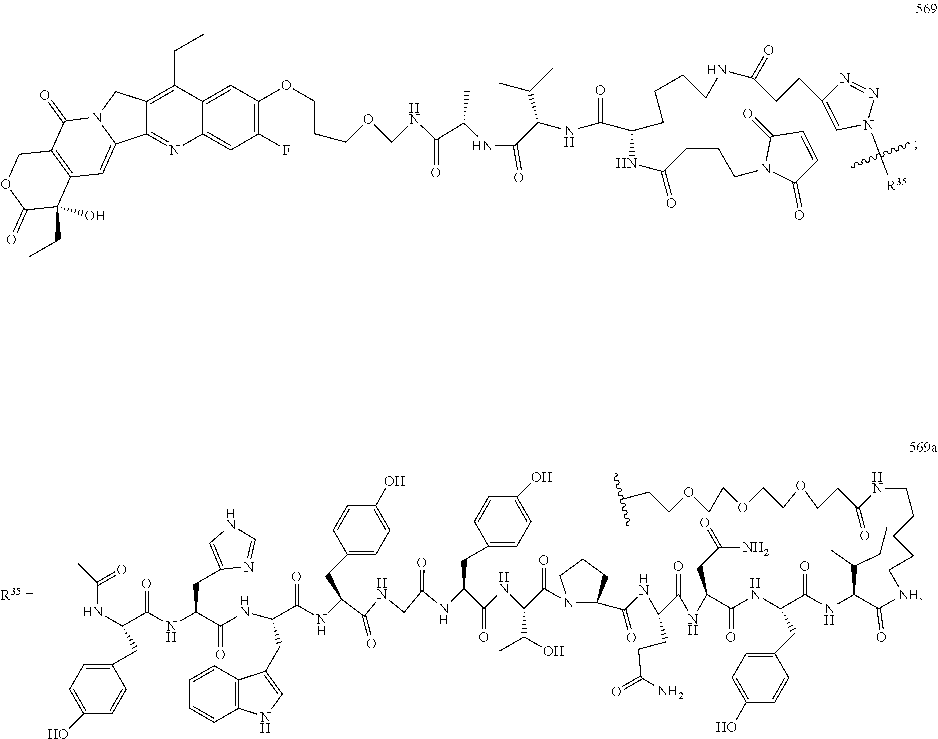

37 mg (48% yield), 95% pure by HPLC, MS-ESI (m/z): [M+H]⁺ calcd for: C₁₄₈H₁₉₂FN₃₀O₃₇, 3000.4049; found:3000.4225.

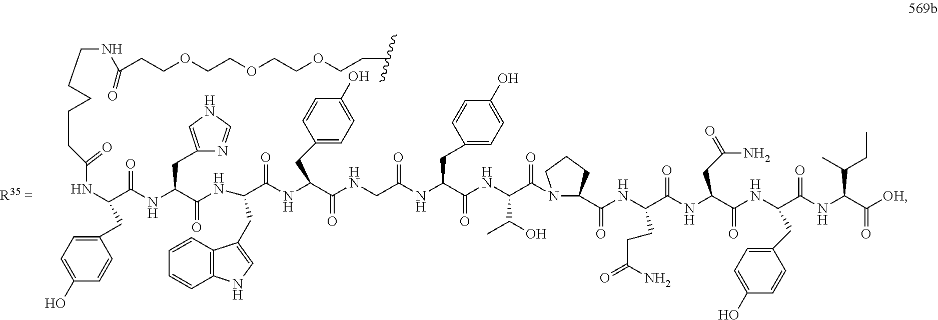 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

32 mg (42% yield), 95% pure by HPLC, MS-ESI (m/z): [M+H]⁺ calcd for: C₁₄₇H₁₈₉FN₂₉O₃₈, 2987.3732; found:2987.3995.

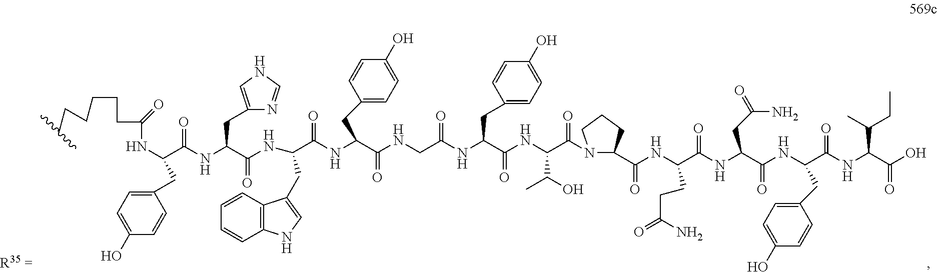 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

31 mg, (41% yield), 95% pure by HPLC, MS-ESI (m/z): [M+H]⁺ calcd for: C₁₃₈H₁₇₂FN₂₈O₃₄, 2784.2575; found:2784.2856.

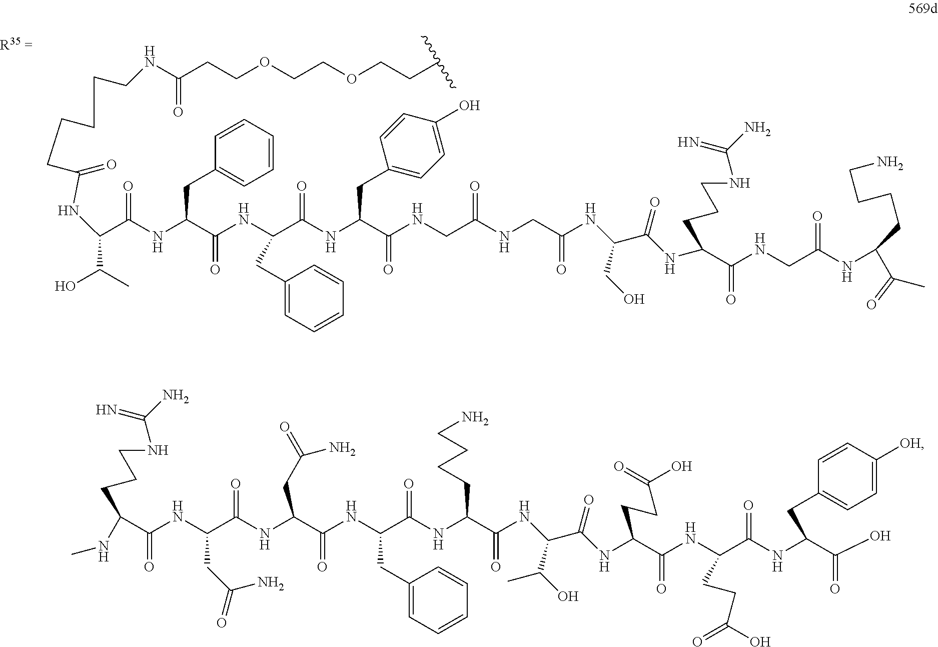

34 mg (36% yield), 93% pure by HPLC, MS-ESI (m/z): [M+H]⁺ calcd for: C₁₇₀H₂₃₇FN₄₁O₄₈, 3639.7349; found:

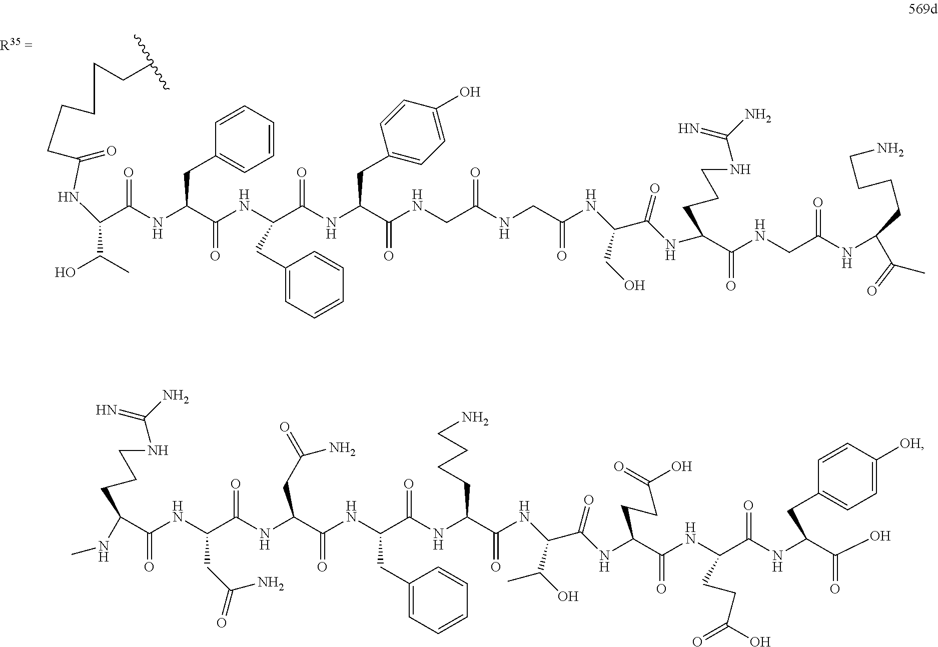

36 mg (40% yield), 95.2% pure, MS, C₁₆₃H₂₂₄FN₄₀O₄₅ [M+H]⁺ 3480.6453; found, 3480.6725;

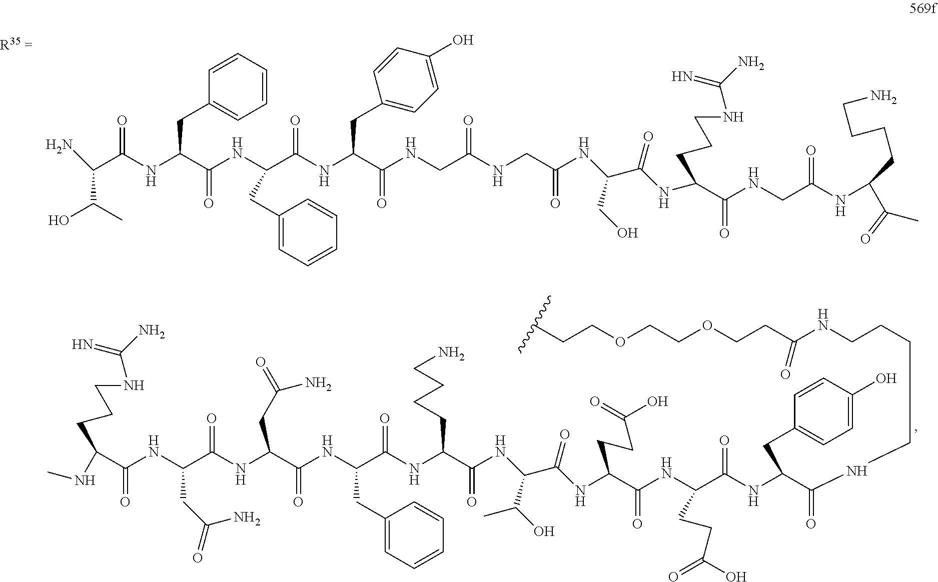

34 mg (37% yield), 95.3% pure; MS, C₁₆₈H₂₃₆FN₄₂O₄₆ [M+H]⁺ 3596.7403; found, 3596.7795;

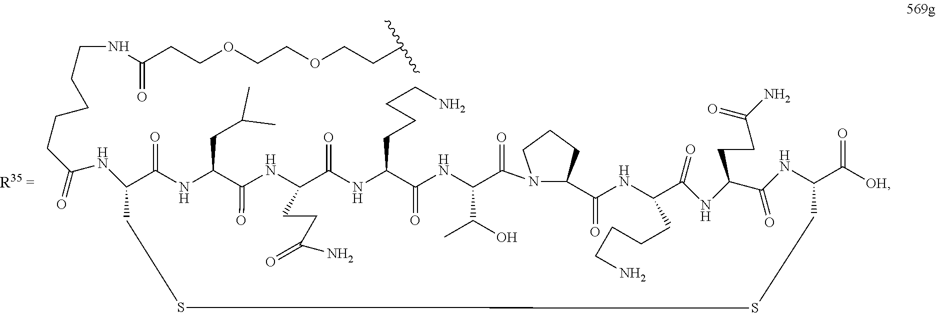 [SMILES: *CCOCCOCCC(=O)NCCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

26 mg (42% yield), 95.7% pure; MS, C₁₀₉H₁₆₃FN₂₅O₃OS₂ [M+H]⁺ 2385.1423; found, 2385.1782;

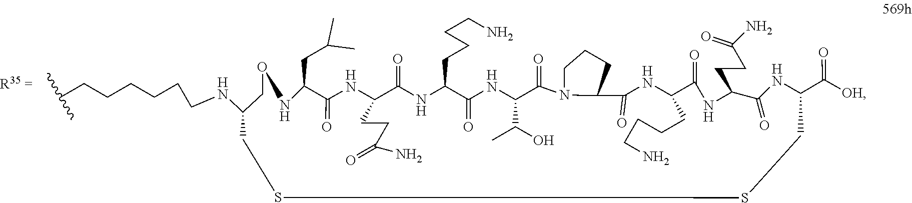

23 mg (40% yield), 94.8% pure by C18-HPLC, MS, C₁₀₂H₁₅FN₂₄O₂₇S₂[M+H]⁺ 2226.0528; found, 2226.0890;

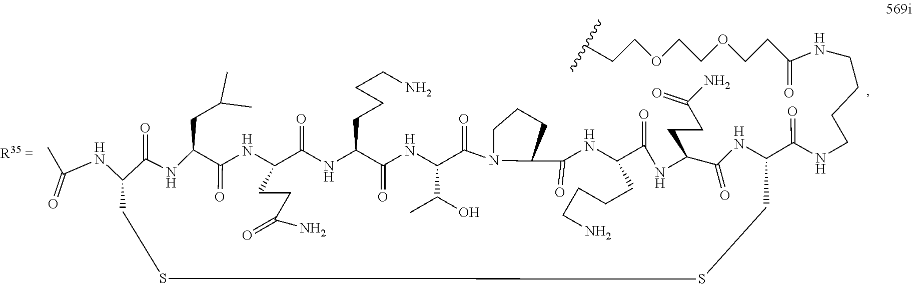

25 mg (41% yield), 95.6% pure by C18-HPLC, MS, C₁₁₉H₁₆₄FN₂₆O₂₉S₂[M+H]⁺ 2384.1583; found, 2384.1855;

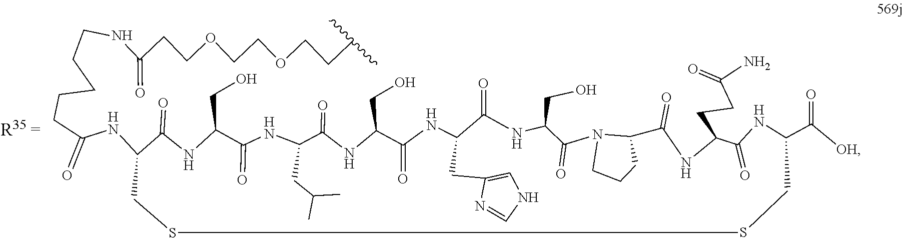

24 mg (41% yield), 95.3% pure by C18-HPLC, MS, C₁₀₃H₁₄₆FN₂₄O₃₁S₂[M+H]⁺ 2298.0011; found, 2298.0375;

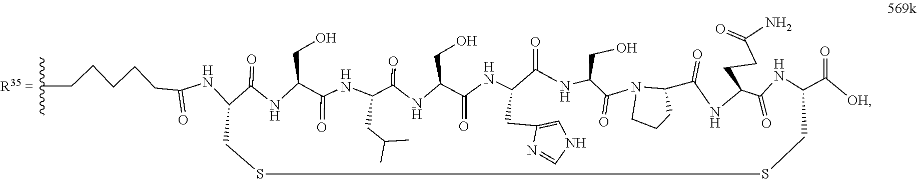

23 mg (40% yield), 95.7% pure by C18-HPLC, MS, C₉₆H₁₃₃FN₂₃O₂₈S₂[M+H]⁺ 2138.9116; found, 2138.9460;

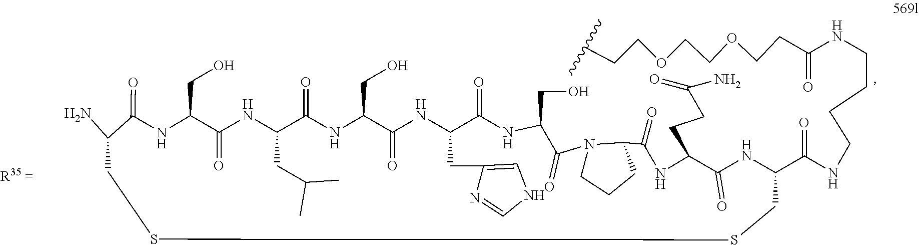

24 mg (41% yield), 95.1% pure by C18-HPLC, MS, C₁₁₀H₁₄₅FN₂₅O₂₉S₂[M+H]⁺ 2255.0065; found, 2255.0393;

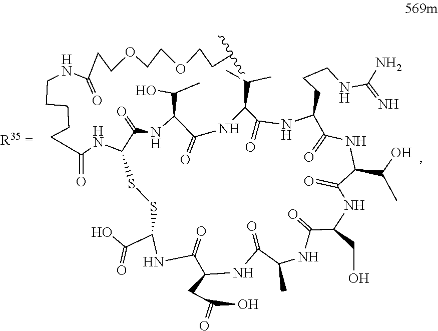

24 mg (41% yield), 95.3% pure by C18-HPLC, MS, C₉₉H₁₄₄FN₂₄O₃₂S₂[M+H]⁺ 2263.9804; found, 2264.0165;

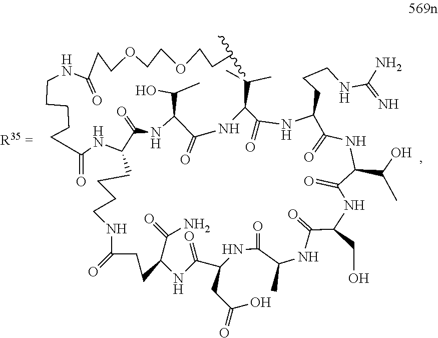

26 mg (44% yield), 95.8% pure by C18-HPLC, MS, C₁₀₆H₁₅₈FN₂₆O₃₂ [M+H]⁺ 2326.1520; found, 2326.1873;

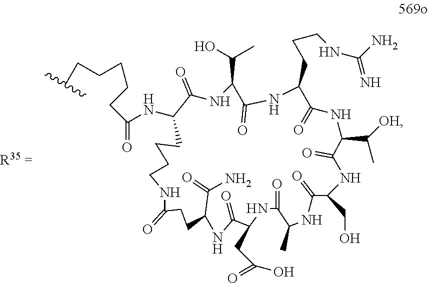 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)O)NC1=O]

28 mg (49% yield), 96.2% pure by C18-HPLC, MS, C₉₉H₁₄₅FN₂₅O₂₉ [M+H]⁺ 2167.0624; found, 2167.0988;

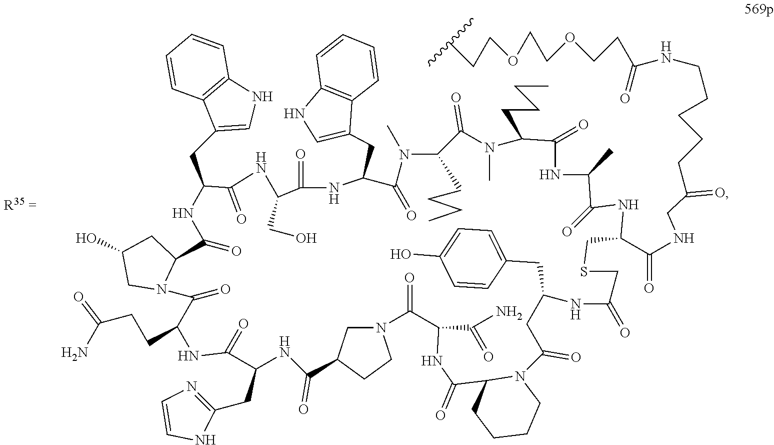 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

35 mg (43% yield), MS, C₁₈₃H₂₀₆FN₃₄O₃₇S [M+H]⁺ 3162.4988; found, 3162.5475;

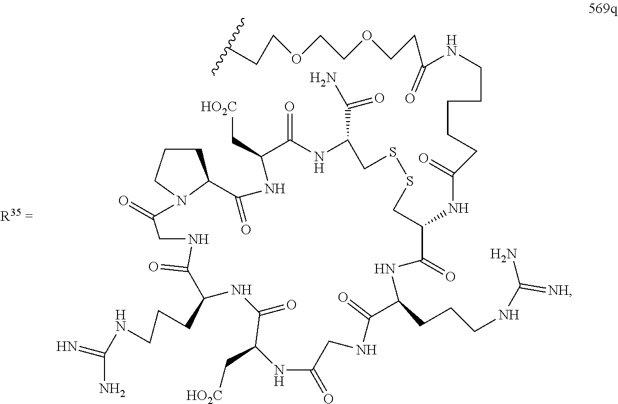 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

26 mg (44% yield), MS, C₁₀₁H₁₄₆FN₂₈O₃OS₂ [M+H]⁺ 2314.0185; found, 2314.0490;

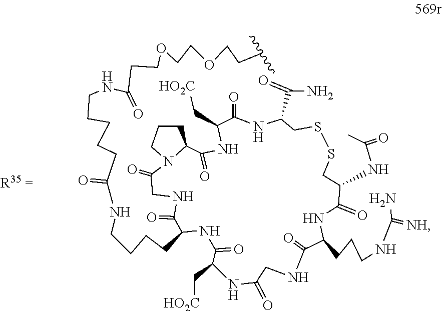 [SMILES: *CCOCCOCCC(=O)NCCCCCC(=O)NCCCC[C@@H]1NC(=O)[C@H](CC(=O)O)NC(=O)CNC(=O)[C@H](CCCNC(=N)N)NC(=O)[C@@H](NC(C)=O)CSSC[C@@H](C(N)=O)NC(=O)[C@H](CC(=O)O)NC(=O)[C@@H]2CCCN2C(=O)CNC1=O]

25 mg (42% yield), MS, C₅₀H₈₃N₁₈O₁₈S₂[M+H]⁺2328.0229; found, 2328.0573;

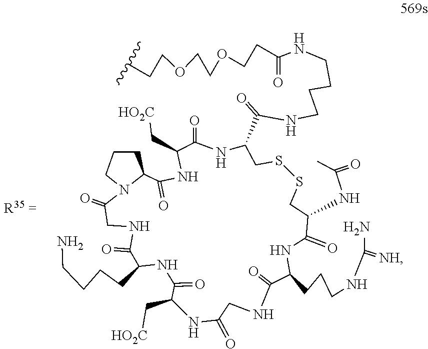 [SMILES: *CCOCCOCCC(=O)NCCCCNC(=O)[C@@H]1CSSC[C@H](NC(C)=O)C(=O)N[C@@H](CCCNC(=N)N)C(=O)NCC(=O)N[C@@H](CC(=O)O)C(=O)N[C@@H](CCCCN)C(=O)NCC(=O)N2CCC[C@H]2C(=O)N[C@@H](CC(=O)O)C(=O)N1]

25 mg (43% yield), MS, C₁₀₁H₁₄₆FN₂₆O₃₀S₂ [M+H]⁺ 2286.0124; found, 2286.0467;

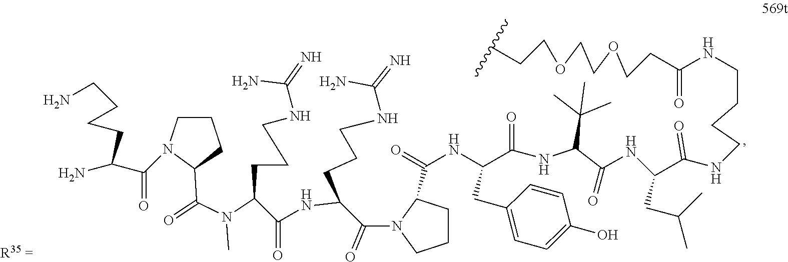

25 mg (42% yield), MS, C₁₁₄H₁₇₂FN₂₈O₂₅ [M+H]⁺ 2352.3032; found, 2352.3382;

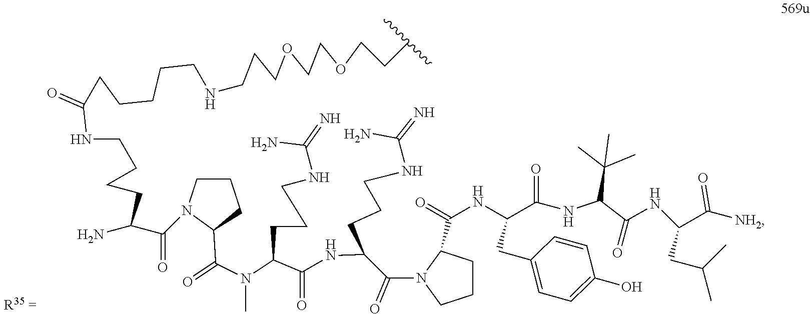

25 mg (41% yield), MS, C₆₃H₁₁₄ N₂₀O₁₂ [M+H]⁺ 2380.3345; found, 2380.3690;

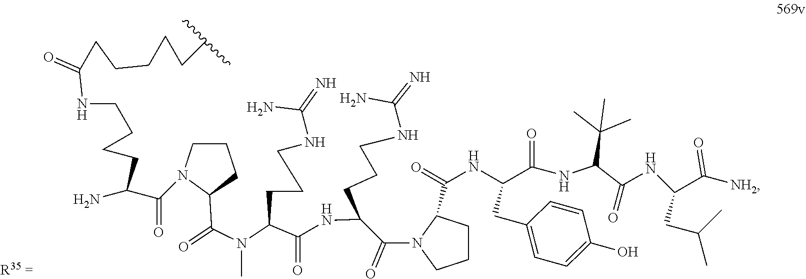

26 mg (44% yield), MS, C₁₁₁H₁₆₃FN₂₇O₂₄ [M+H]⁺ 2277.2348; found, 2277.2677;

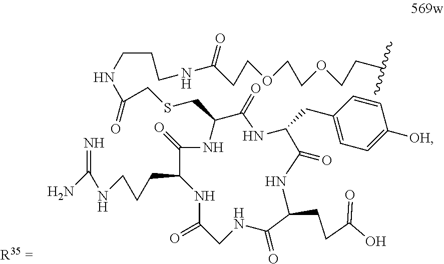 [SMILES: *CCOCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

25 mg (49% yield), MS, C₉₀H₁₂₃FN₂₁O₂₅S [M+H]⁺ 1948.8704; found, 1948.9008;

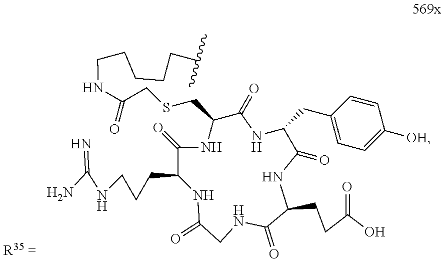 [SMILES: *CCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

26 mg (55% yield), MS, C₈₅H₁₁₄FN₂₀O₂₂S [M+H]⁺ 1817.8121; found, 1817.8425;

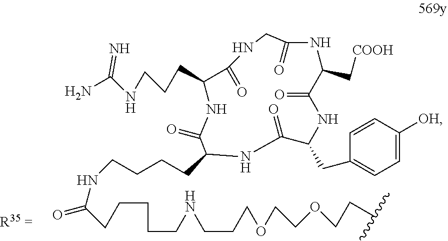 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

26 mg (52% yield), MS, C₉₃H₁₃₁FN₂₁O₂₄ [M+H]⁺ 1944.9660; found, 1944.9992;

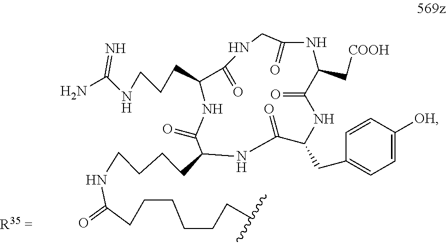 [SMILES: *CCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

26.6 mg (57% yield), MS, C₈₆H₁₁₆FN₂₀O₂₂ [M+H]⁺ 1799.8557; found, 1799.8965.

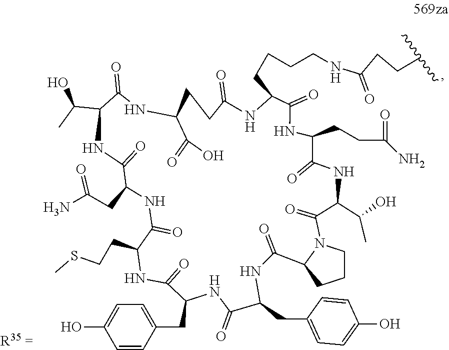 [SMILES: *CCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

28.3 mg (46% yield), MS, C₁₂H₁₅₀FN₂₄O₃₂S [M+H]⁺ 2394.0553; found, 2394.0898.

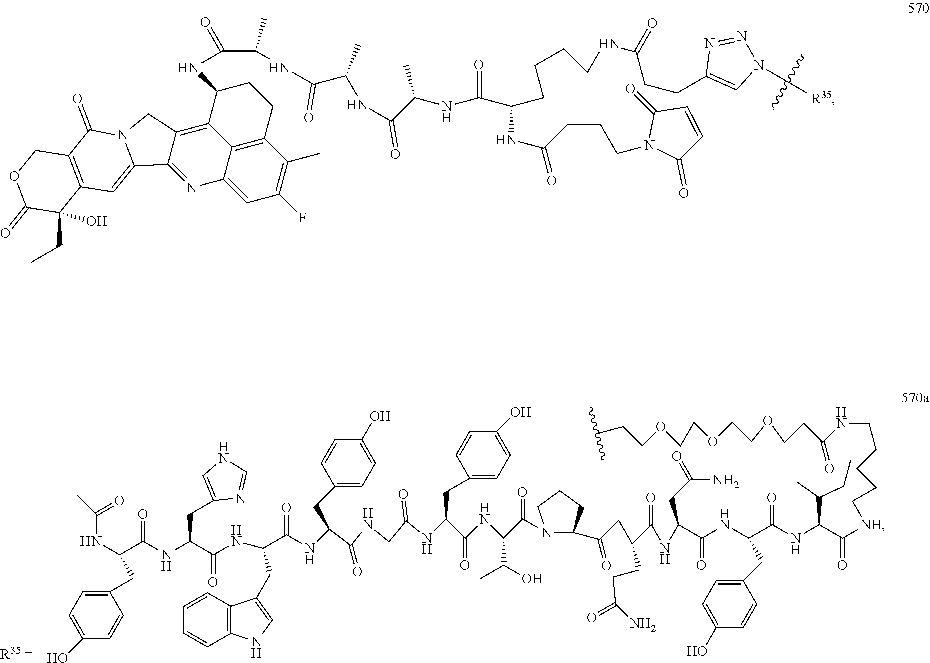

37 mg (45% yield), 95% pure by HPLC, MS-ESI (m/z): [M+H]⁺ calcd for: C₁₄₇H₁₈₇FN₃₁O₃₆, 2981.3739; found: 2981.4058.

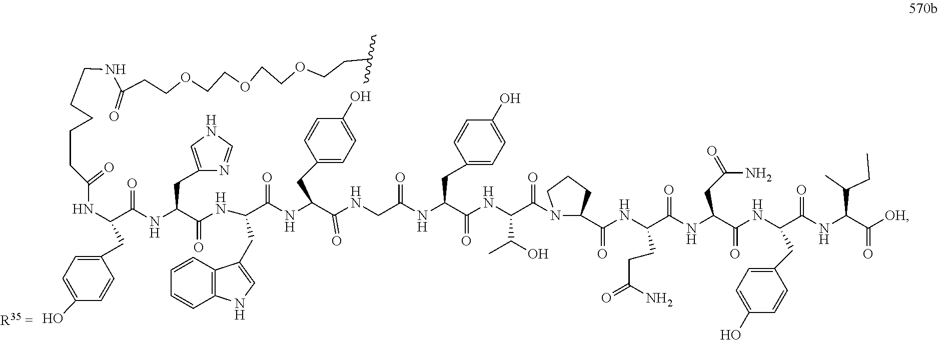 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

36 mg (44% yield), 95% pure by HPLC, MS-ESI (m/z): [M+H]⁺ calcd for: C₁₄₆H₁₈₃FN₃₀O₃₇, 2967.3334; found:2967.3693.

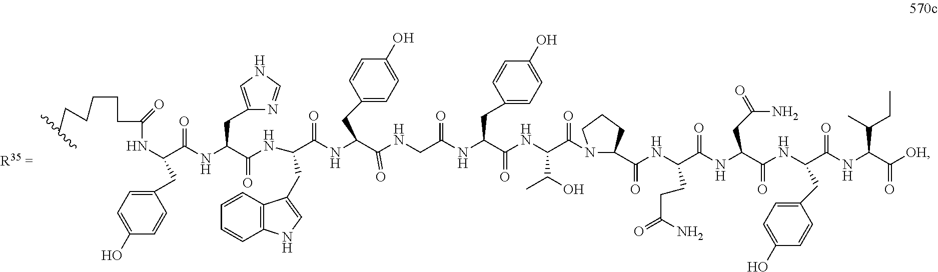 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

31 mg, (41% yield), 95% pure by HPLC, MS-ESI (m/z): [M+H]⁺ calcd for: C₁₃₇H₁₆₇FN₂₉O₃₃, 2765.2265; found:2767.2295.

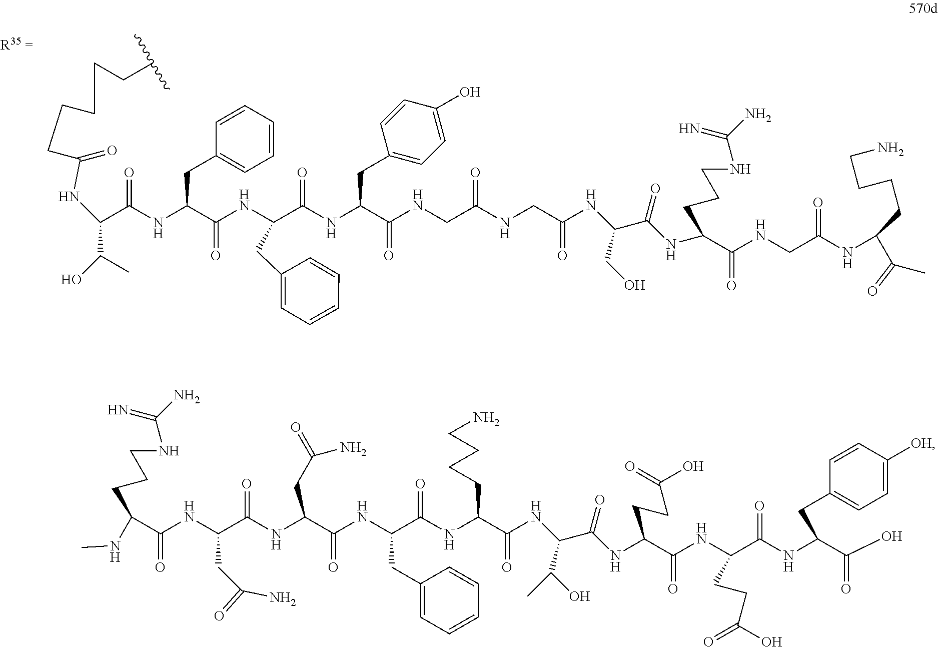

36 mg (40% yield), 95.2% pure, MS, C₁₆₂H₂₁₈FN₄₁O₄₄ [M+H]⁺ 3460.6065; found, 3460.6408;

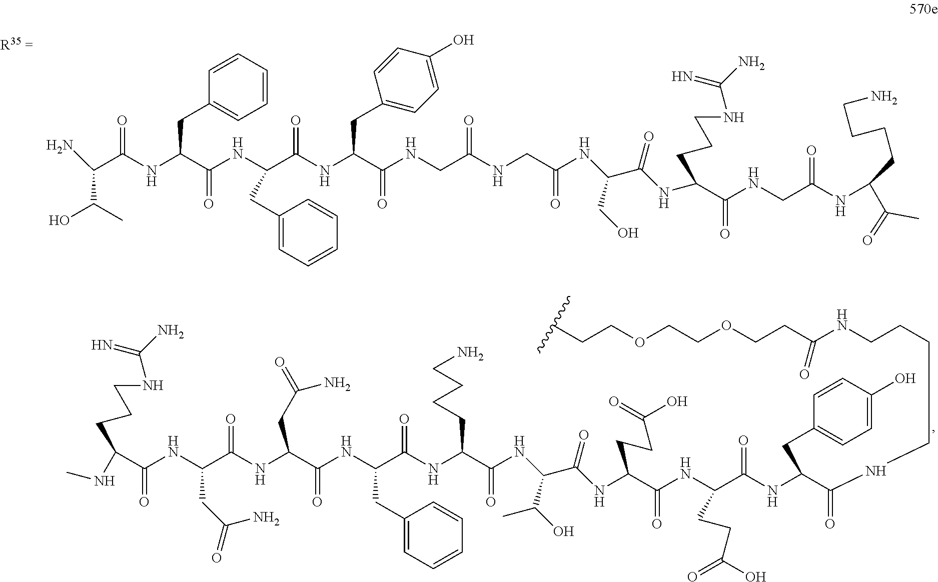

34 mg (37% yield), 95.3% pure; MS, C₁₆₈H₂₃₆FN₄₂O₄₆ [M+H]⁺ 3596.7403; found, 3596.7795;

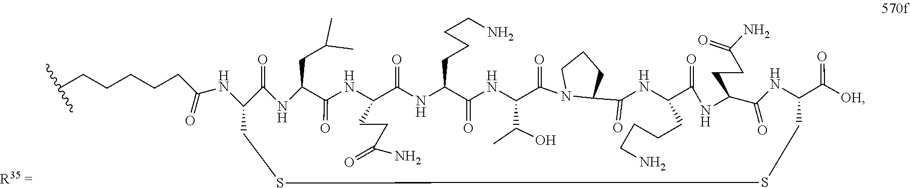 [SMILES: *CCCCCC(=O)N[C@H]1CSSC[C@@H](C(=O)O)NC(=O)[C@H](CCC(N)=O)NC(=O)[C@H](CCCCN)NC(=O)[C@H]2CCCN2C(=O)[C@H](C(C)O)NC(=O)[C@H](CCCCN)NC(=O)[C@H](CCC(N)=O)NC(=O)[C@H](CC(C)C)NC1=O]

23 mg (40% yield), 94.8% pure by C18-HPLC, MS, C₁₀₁H₁₄₄FN₂₅O₂₆S₂[M+H]⁺ 2206.0140; found, 2206.0396;

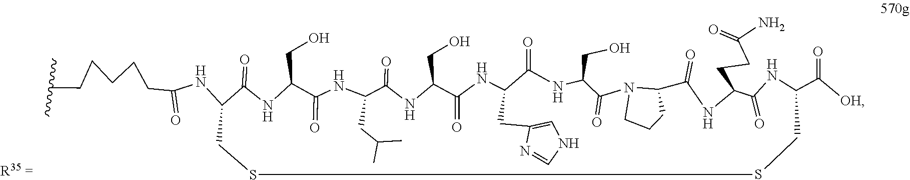

23 mg (39% yield), 95.4% pure by C18-HPLC, MS, C₉₅H₁₂₈FN₂₄O₂₇S₂[M+H]⁺ 2119.8806; found, 2119.9167;

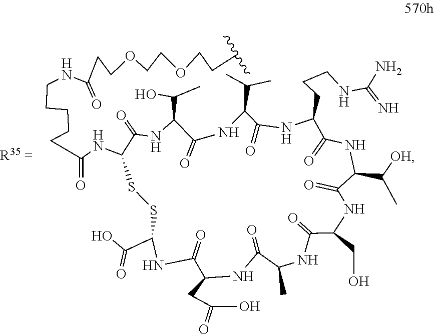 [SMILES: *CCOCCOCCC(=O)NCCCCCC(=O)N[C@H]1SS[C@@H](C(=O)O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

24 mg (40% yield), 95.4% pure by C18-HPLC, MS, C₉₈H₁₃₉FN₂₅O₃₁S₂ [M+H]⁺2244.9494; found, 2C244.9836;

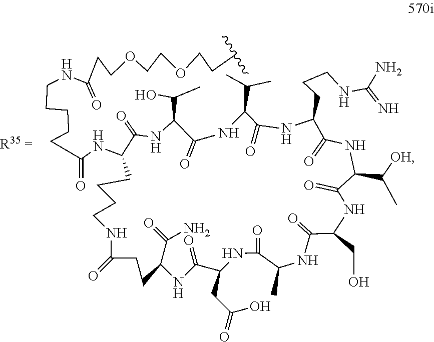 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

26 mg (410% yield), 96.9% pure by C18-HPLC, MS, C₁₀₅H₁₅₃FN₂₇O₃₁ [M+H]⁺ 2307.1210; found, 2307.1569;

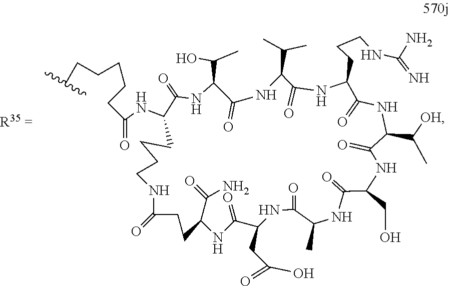 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

28 mg (49% yield), 96.3% pure by C18-HPLC, MS, C₉₈H₁₃₉FN₂₆O₂₈ [M+H]⁺ 2147.0236; found, 2147.0573;

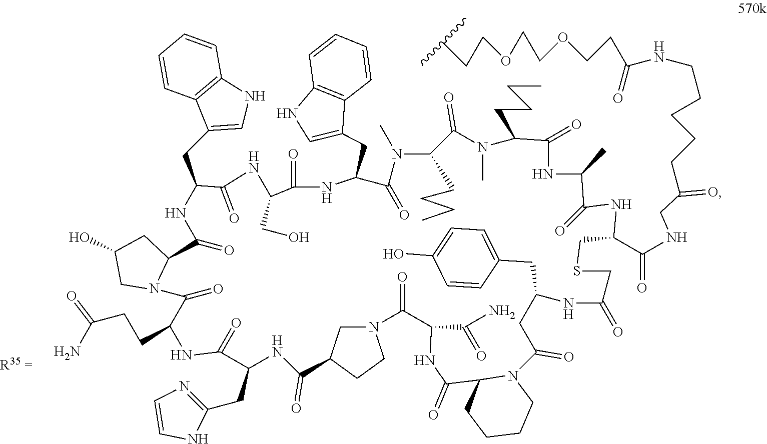 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

27 mg (34% yield), 96.1% pure by C18-HPLC, MS, C₁₈₂H₂₀₁FN₃₈O₃₆S [M+H]⁺ 31434678; found, 3143.4997;

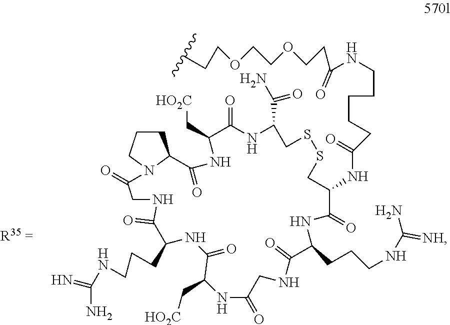 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

26 mg (45% yield), 95.3% pure by C18-HPLC, MS, C₁₀₀H₁₄₁FN₂₉O₂₉S₂[M+H]⁺ 2294.9875; found, 2295.0205;

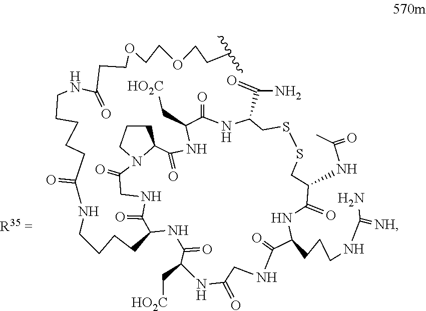 [SMILES: *CCOCCOCCC(=O)NCCCCCC(=O)NCCCC[C@@H]1NC(=O)[C@H](CC(=O)O)NC(=O)CNC(=O)[C@H](CCCNC(=N)N)NC(=O)[C@@H](NC(C)=O)CSSC[C@@H](C(N)=O)NC(=O)[C@H](CC(=O)O)NC(=O)[C@@H]2CCCN2C(=O)CNC1=O]

25 mg (43% yield), 95.1% pure by C18-HPLC, MS, MS, C₁₀₂H₁₄₂FN₂₇O₃₅₂ [M+H]⁺ 2307.9841; found, 2308.0185;

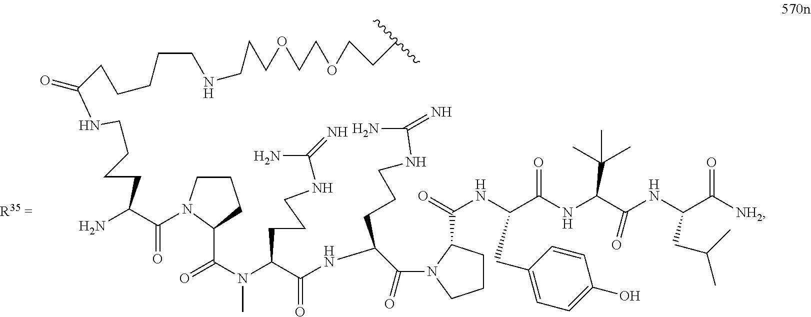 [SMILES: *CCOCCOCCCNCCCCCC(=O)NCCCC[C@H](N)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(N)=O)C(C)(C)C]

25 mg (41% yield), 96.3% pure by C18-HPLC, MS, C₁₁₅H₁₇₁FN₂₉O₂₄ [M+H]⁺ 2361.3036; found, 2361.3386;

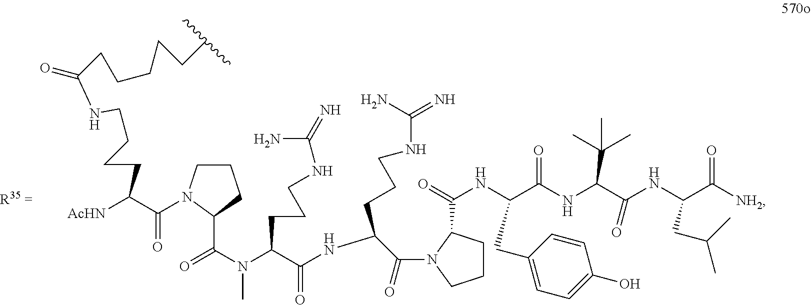

26 mg (42% yield), 96.3% pure by C18-HPLC, MS, C₁₁₀H₁₅₈FN₂₈O₂₃ [M+H]⁺ 2258.2039; found, 2258.2373;

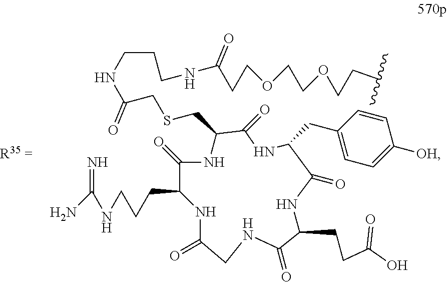 [SMILES: *CCOCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

26 mg (45% yield), 96.3% pure by C18-HPLC, MS, Cs₉H₁₁₈FN₂₂O₂₄S [M+H]⁺ 1929.8394; found, 1929.8732;

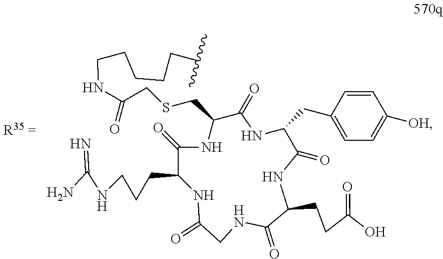 [SMILES: *CCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

26 mg (45% yield), 96.6% pure by C18-HPLC, MS, C₈₄H₁₀₉FN₂₁O₂₁S [M+H]⁺ 1798.7812; found, 1798.8125;

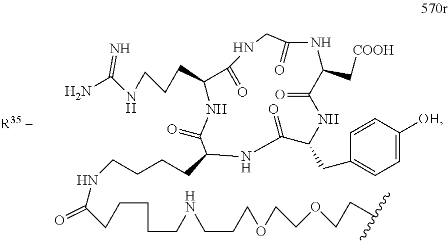 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

25 mg (47% yield), 95.3% pure by C18-HPLC, MS, C₉₂H₁₂₆FN₂₂O₂₃ [M+H]⁺ 1925.9350; found, 1925.9695;

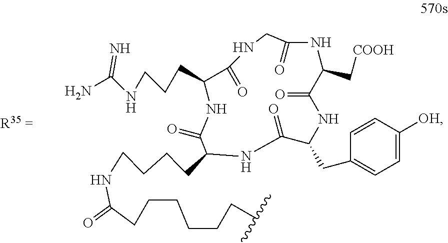 [SMILES: *CCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

26 mg (53% yield), 96.4% pure by C18-HPLC, MS, C₈₆H₁₁₃FN₂₁O₂₁ [M+H]⁺ 1794.8404; found, 1794.8750.

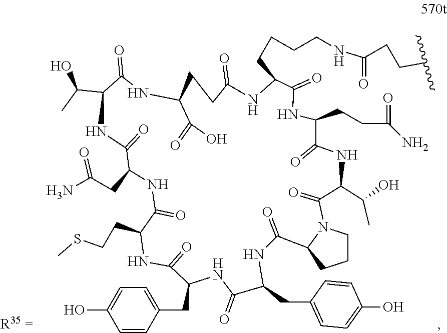 [SMILES: *CCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

30.6 mg (46% yield), MS, C₁₁₁H₄₅FN₂₅O₃₁S [M+H]⁺ 2375.0243; found, 2375.0567.

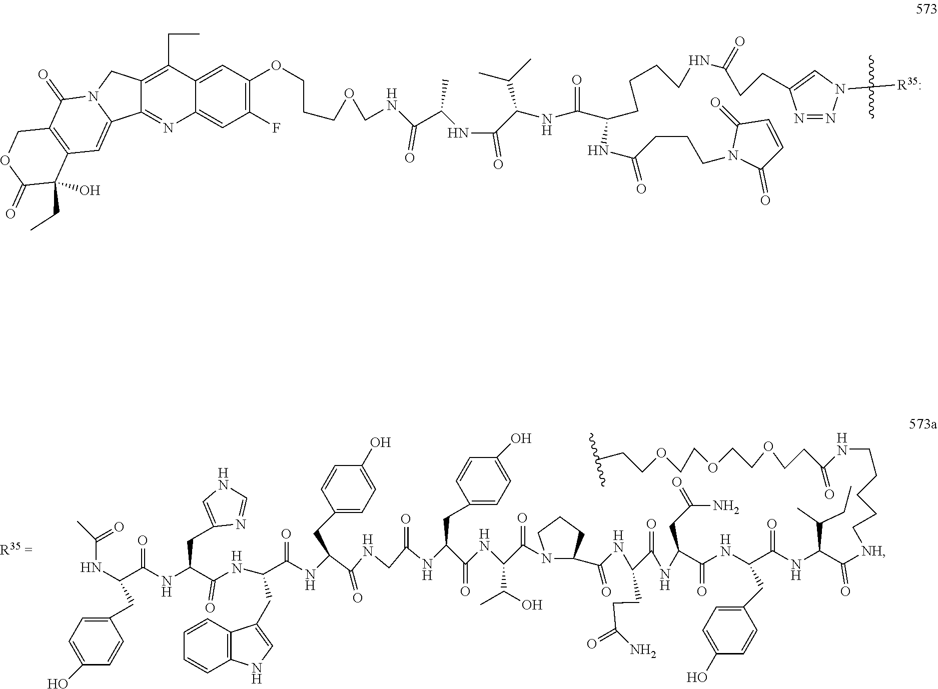

31 mg (60% yield), 95.4% pure by HPLC, MS-ESI (m/z): [M+2H]²⁺ calcd for: C₁₄₈H₁₉₃FN₃₀O₃₇, 1500.7063; found: 1500.7325.

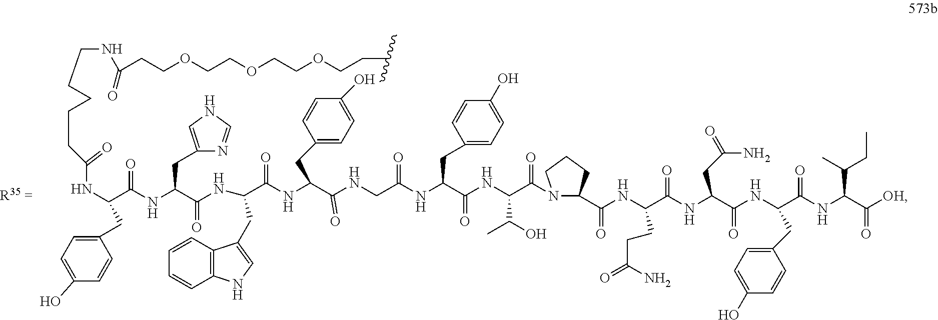 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

33 mg (64% yield), 95.1% pure by HPLC, MS-ESI (m/z): [M+2H]²⁺ calcd for: C₁₄₇H₁₉₀FN₂₉O₃₈, 1494.1905; found: 1494.1905.

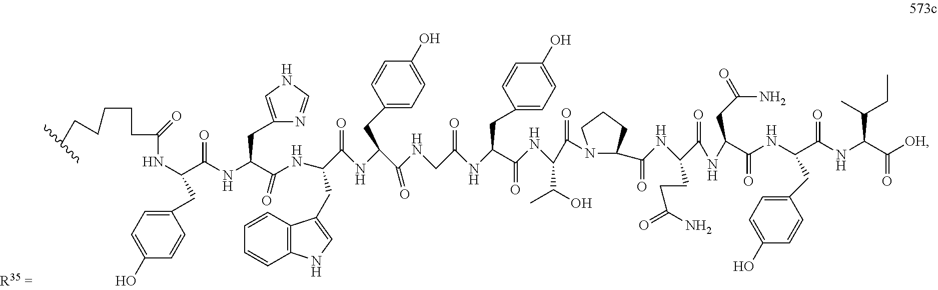 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

35 mg, (73% yield), 95.2% pure by HPLC, MS-ESI (m/z): [M+2H]²⁺ calcd for: C₁₃₈H₁₇₃FN₂₈O₃₄, 1392.6326; found: 1392.6593.

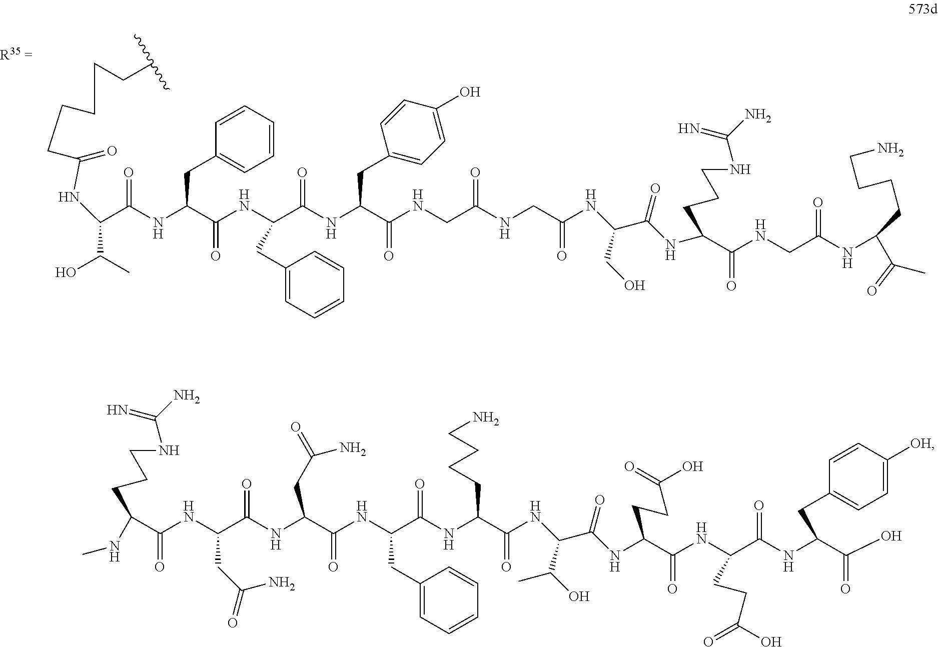

33 mg (55% yield), 94.3% pure, MS-ESI (m/z): [M+2H]²⁺+calcd for: C₁₆₃H₂₂₅FN₄₀O₄₅, 1740.8266; found: 1740.8587.

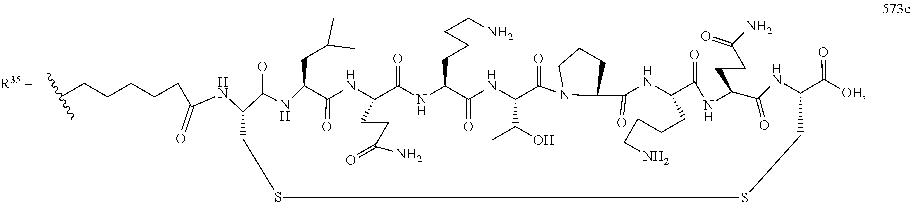

29 mg (76% yield), 95.5% pure by C18-HPLC, MS m/z, C₁₀₂H₁₅₀FN₂₄O₂₇S₂[M+H]⁺ 2226.0528; found, 2226.0885;

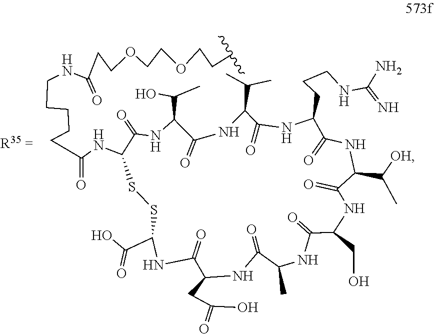 [SMILES: *CCOCCOCCC(=O)NCCCCCC(=O)N[C@H]1SS[C@@H](C(=O)O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

25 mg (64% yield), 95.2% pure by C18-HPLC, MS, C₉₉H₁₄₄FN₂₄O₃₂S₂[M+H]⁺ 2263.9804 found, 2264.0281;

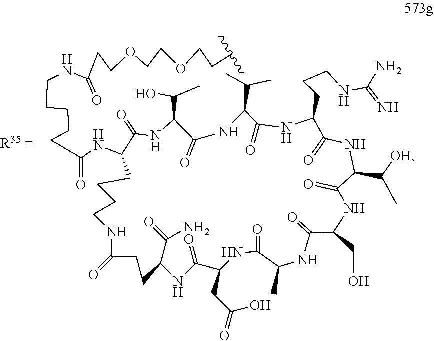 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

29 mg (72% yield), 96.1% pure by C18-HPLC, MS m/z, C₁₀₄H₁₅₈FN₂₆O₃₂ [M+H]⁺ 2326.1520; found, 2326.15875;

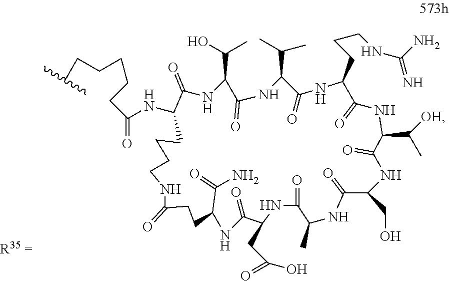 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

30 mg (80% yield), 96.9% pure by C18-HPLC, MS m/z, C₉₉H₁₄₅FN₂₅O₂₉ [M+H]⁺ 2167.0624; found, 2167.0967;

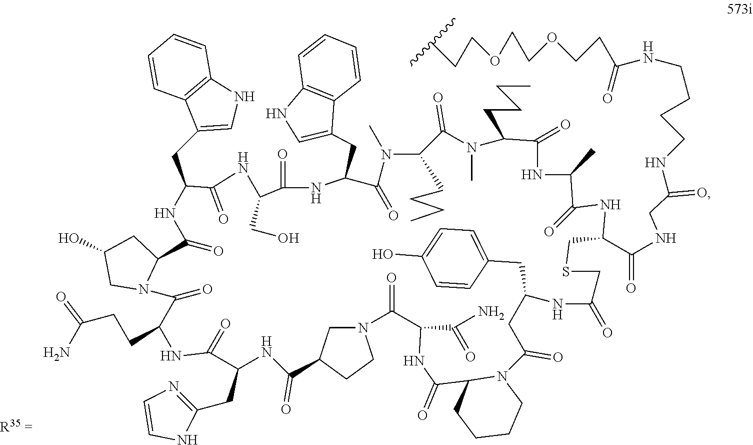 [SMILES: *CCOCCOCCC(=O)NCCCCNC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

32 mg (58% yield), 95.5% pure by C18-HPLC, MS m/z, C₁₅₄H₂₀₉FN₃₄O₃₇S [M+2H]²⁺ 1588.7611; found, 1588.7905;

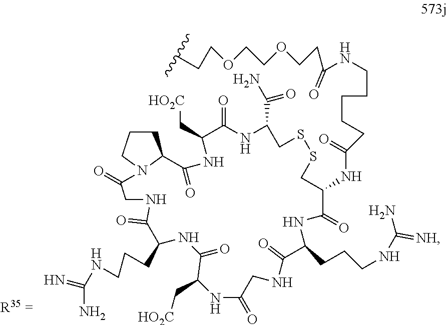 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

27 mg (68% yield), 94.7% pure by C18-HPLC, MS, C₁₀₁H₁₄₆FN₂₈O₃₀S₂ [M+H]⁺ 2314.0185; found, 2314.0433;

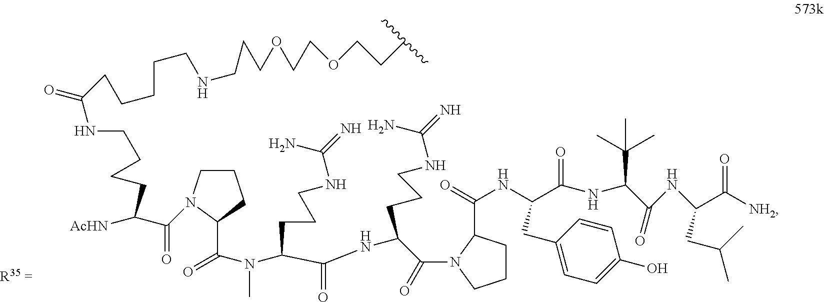 [SMILES: *CCOCCOCCCNCCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCCC1C(=O)N[C@@H](Cc1ccc(O)cc1)C(=O)N[C@H](C(=O)N[C@@H](CC(C)C)C(N)=O)C(C)(C)C]

29 mg (70% yield), 96.1% pure by C18-HPLC, MS m/z, C₁₁₈H₁₇₈FN₂₈O₂₆ [M+H]⁺ 2422.3451; found, 2422.3709;

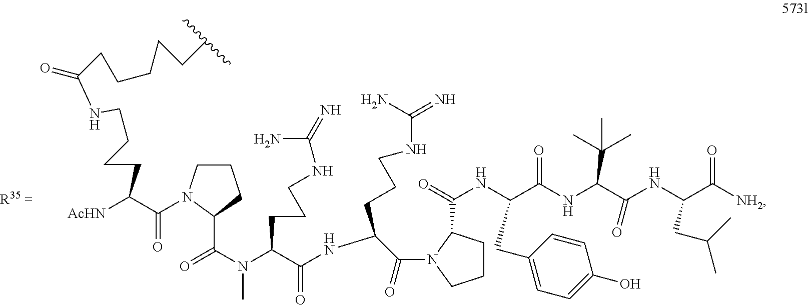

28 mg (72% yield), 96.5% pure by C18-HPLC, MS m/z, C₁₁₁H₁₆₃FN₂₇O₂₄ [M+H]⁺ 2277.2348; found, 2277.2686;

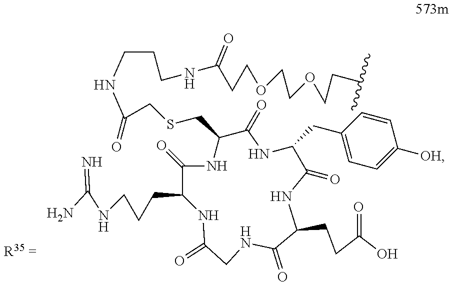 [SMILES: *CCOCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

27 mg (81% yield), 95.1% pure by C18-HPLC, MS m/z, C₉₀H₁₂₃FN₂₁O₂₅S [M+H]⁺ 1948.8704; found, 1948.9055;

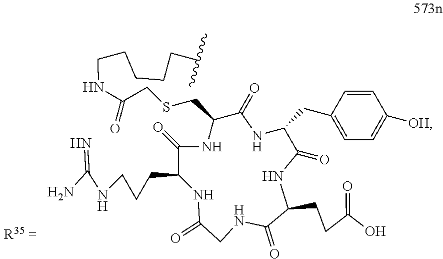 [SMILES: *CCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

26 mg (81% yield), 95.0% pure by C18-HPLC, MS m/z, C₈₅H₁₁₄FN₂₀O₂₂S [M+H]⁺ 1817.121; found, 1817.8436;

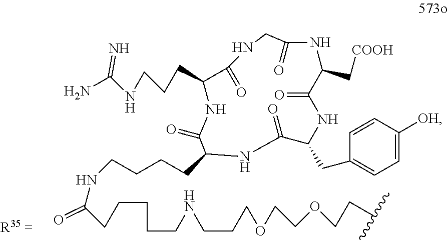 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

23 mg (68% yield), 95.1% pure by C18-HPLC, MS m/z, C₉₄H₁₃₃FN₂₁O₂₄ [M+H]⁺ 1958.9816; found, 1959.0085;

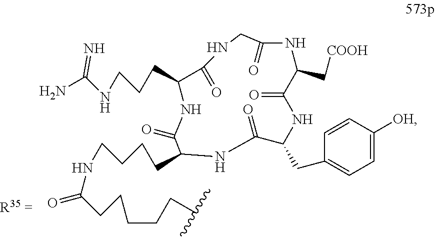 [SMILES: *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

22 mg (70% yield), 96.8% pure by C18-HPLC, MS m/z, C₈₇H₁₁₈FN₂₀O₂₂ [M+H]⁺ 1813.8714; found, 1813.8966.

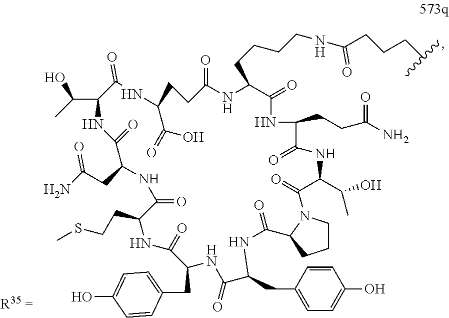 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

30 mg (72% yield), 95.1% pure by C18-HPLC, MS, C₁₁₃H₁₅₂FN₂₄O₃₂S [M+H]⁺ 2408.0709; found, 2408.1015.

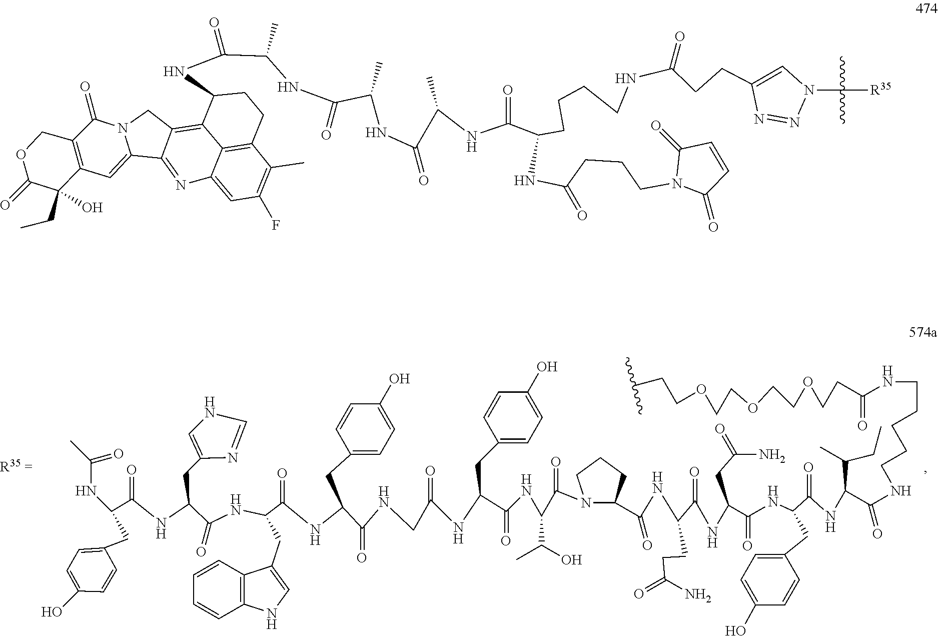

32 mg (61o yield), 95.1% pure by HPLC, MS-ESI (m/z): [M+2H]²⁺ calcd for: C₁₄₇H₁₈₇FN₃ O₃₆, 1490.6869; found: 1490.7097.

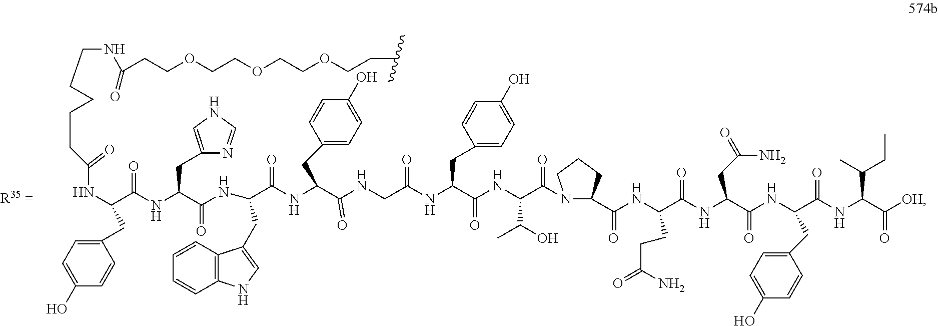 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

34 mg (6700 yield), 95.5% pure by HPLC, MS-ESI (m/z): [M+2H]²⁺ calcd for: C₁₄₆H₁₈₄FN₃₀O₃₇, 1484.1711; found: 1484.1983.

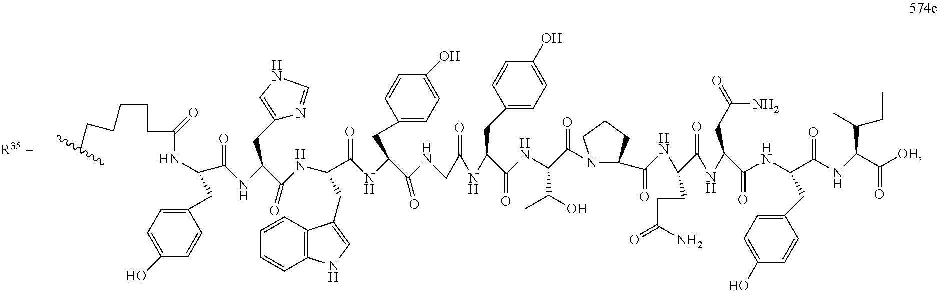 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

35 mg, (74% yield), 95.3% pure by HPLC, MS-ESI (m/z): [M+2H]²⁺ calcd for: C₄₃₇H₁₀₇FN₂₉O₃₃, 1382.6132; found: 1382.6396.

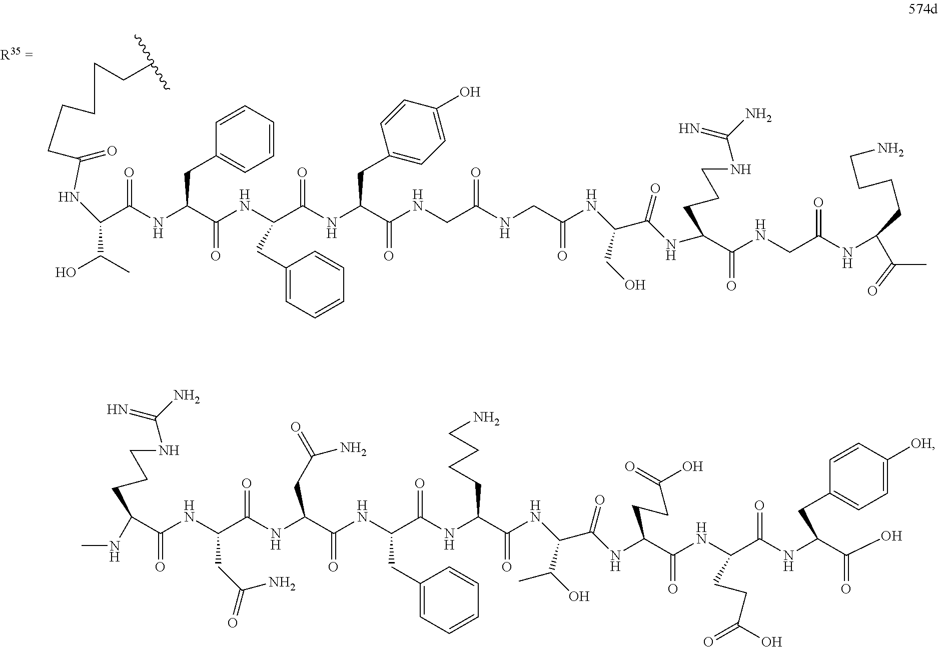

34 mg (57% yield), 94.3% pure, MS-ESI (m/z): [M+2H]²⁺+calcd for: C₁₁₂H₂₁₉FN₄₁O₄₄, 1730.0807; found: 1730.1048.

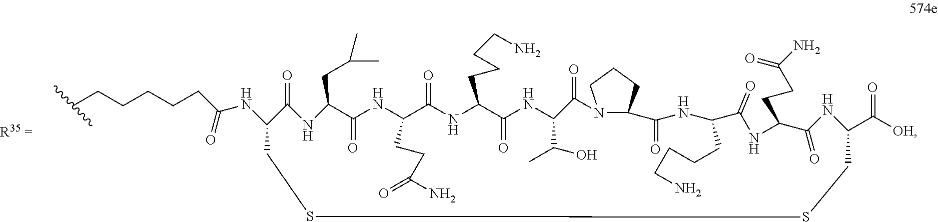 [SMILES: *CCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

29 mg (76% yield), 95.2% pure by C18-HPLC, MS m/z, C₁₀₁H₁₄₅FN₂₅O₂₆S₂[M+H]⁺ 2207.0218; found, 2207.0445;

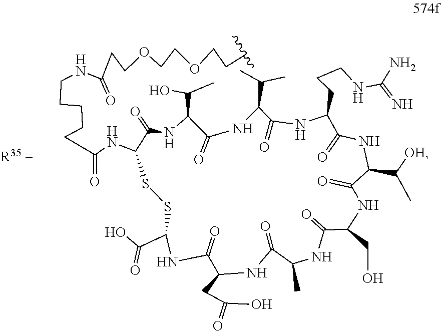

25 mg (65% yield), 95.0% pure by C18-HPLC, MS, C₉₈H₁₃₉FN₂₅O₃₁S₂ [M+H]⁺ 2244.9494 found, 2244.9712;

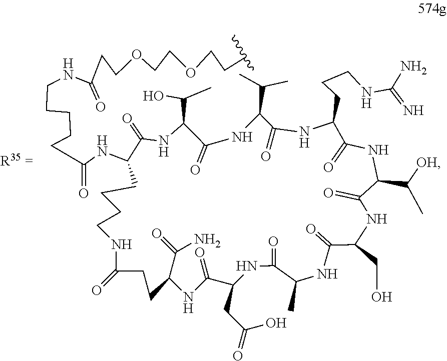 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

28 mg (71% yield), 96.1% pure by C18-HPLC, MS m/z, C₁₀₅H₁₅₃FN₂₇O₃₁ [M+H]⁺ 2307.1210; found, 2307.1457;

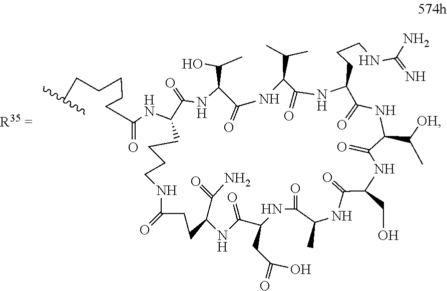

28 mg (76% yield), 96.4% pure by C18-HPLC, MS m/z, C₉₈H₁₄₀FN₂₆O₂₈ [M+H]⁺ 2148.0314; found, 2148.0573;

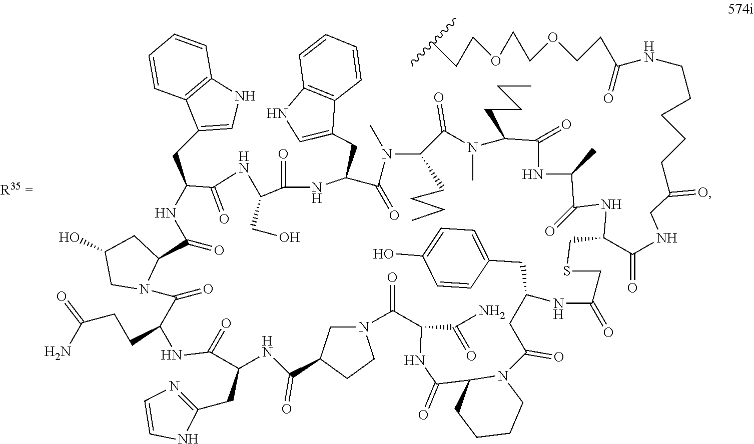 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

33 mg (61% yield), 95.5% pure by C18-HPLC, MS m/z, C₁₅₂H₂₀₁FN₃₅O₃₆S [M+2H]²⁺ 1571.7339; found, 1571.7567;

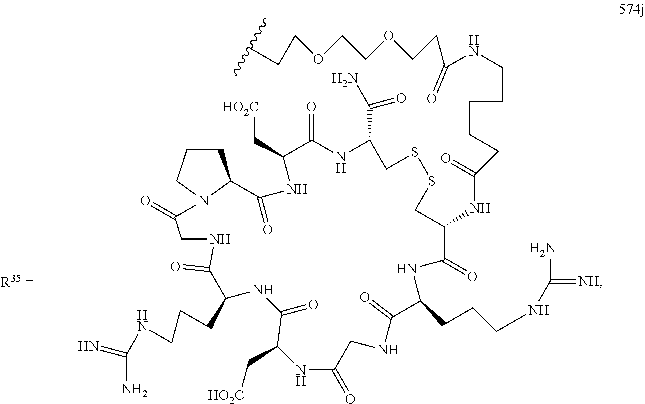 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

25 mg (63% yield), 94.7% pure by C18-HPLC, MS, C₁₀₀H₁₄₁FN₂₉O₂₉S₂[M+H]⁺ 2294.9875; found, 2295.0115;

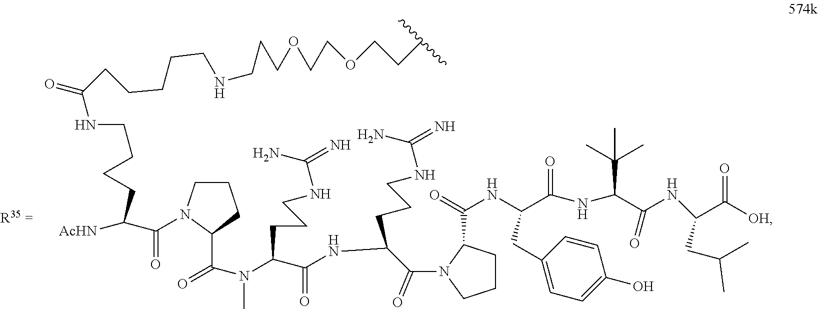 [SMILES: *CCOCCOCCCNCCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

28 mg (68% yield), 95.5% pure by C18-HPLC, MS m/z, C₁₁₇H₁₇₂FN₂₈O₂₆ [M+H]⁺ 2404.29821; found, 2404.3110;

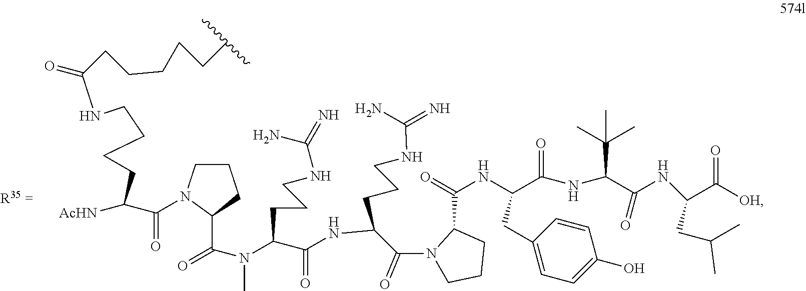 [SMILES: *CCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

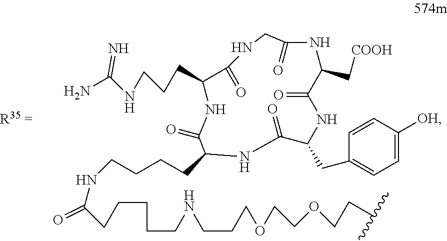 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

23 mg (70% yield), 95.0% pure by C18-HPLC, MS m/z, C₉₄H₁₂₆FN₂₂O₂₃ [M+H]⁺ 1925.9350; found, 1925.9595

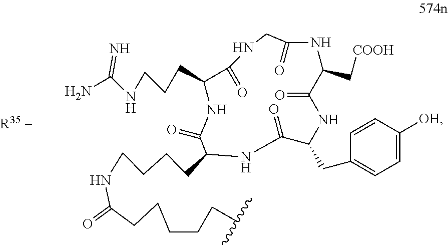 [SMILES: *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

21 mg (690% yield), 96.30% pure by C18-HPLC, MS m/z, C₈₅H₁₁₂FN₂₁O₂₁ [M+H]⁺ 1780.8247; found, 1780.8598.

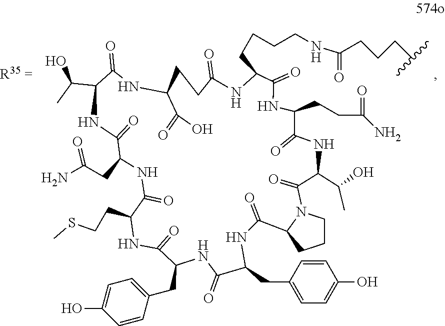 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

29 mg (71% yield), 95.2% pure by C18-HPLC, MS, C₁₁₂H₁₄₇FN₂₅O₃₁S [M+H]⁺ 2389.0400; found, 2389.0719.

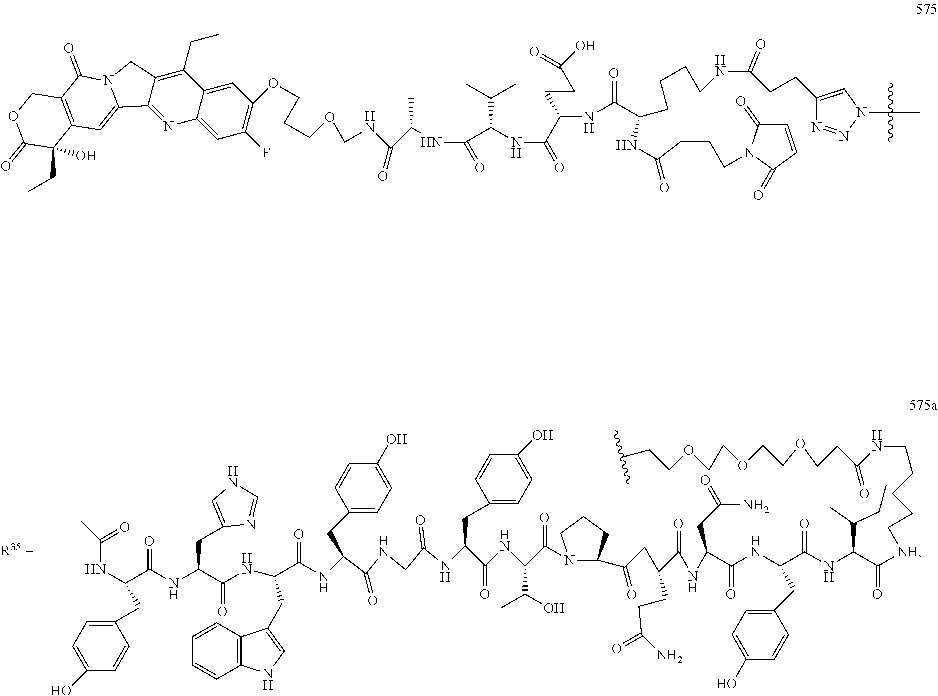 [SMILES: *n1cc(CCC(=O)NCCCC[C@H](NC(=O)CCCN2C(=O)C=CC2=O)C(=O)N[C@@H](CCC(=O)O)C(=O)N[C@H](C(=O)N[C@@H](C)C(=O)NCOCCCOc2cc3c(CC)c4c(nc3cc2F)-c2cc3c(c(=O)n2C4)COC(=O)[C@]3(O)CC)C(C)C)nn1 ; *CCOCCOCCOCCC(=O)NCCCCCNC(=O)[C@@H](NC(=O)[C@H](Cc1ccc(O)cc1)NC(=O)[C@H](CC(N)=O)NC(=O)[C@H](CCC(N)=O)CC(=O)[C@@H]1CCCN1C(=O)[C@@H](NC(=O)[C@H](Cc1ccc(O)cc1)NC(=O)CNC(=O)[C@H](Cc1ccc(O)cc1)NC(=O)[C@H](Cc1c[nH]c2ccccc12)NC(=O)[C@H](Cc1c[nH]cn1)NC(=O)[C@H](Cc1ccc(O)cc1)NC(C)=O)C(C)O)C(C)CC]

31 mg (58% yield), 95.4% pure by HPLC, MS-ESI (m/z): [M+2H]²⁺ calcd for: C₁₅₃H₁₉₉FN₃₁O₄₀, 1565.2276; found: 1565.2515;

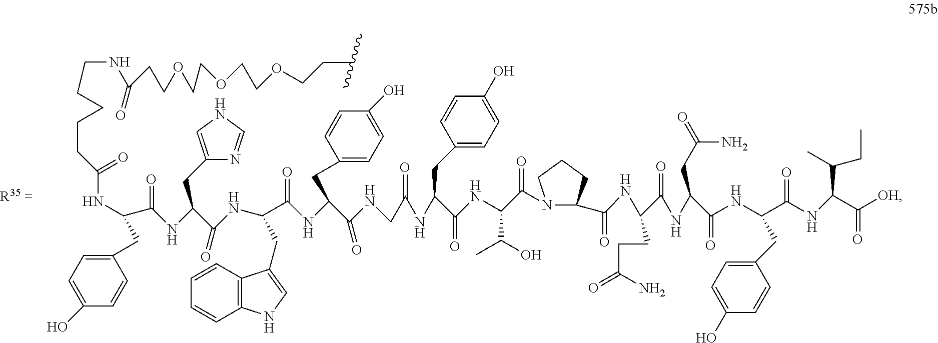 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

32 mg (5900 yield), 95.3% pure by HPLC, MS-ESI (m/z): [M+2H]²⁺ calcd for: C₁₈₃H₁₉₈FN₃₀O₄₁, 1565.7196; found: 1565.7412;

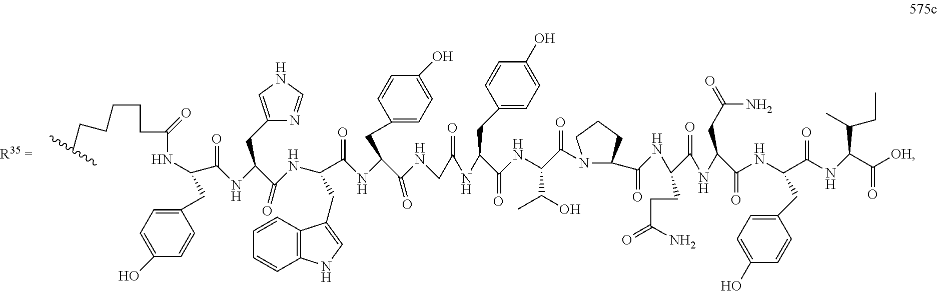 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

34 mg, (68% yield), 95.5% pure by HPLC, MS-ESI (m/z): [M+2H]²⁺ calcd for: C₄H₁₇₉FN₂₉O₃₇, 1457.1539; found: 1457.1782;

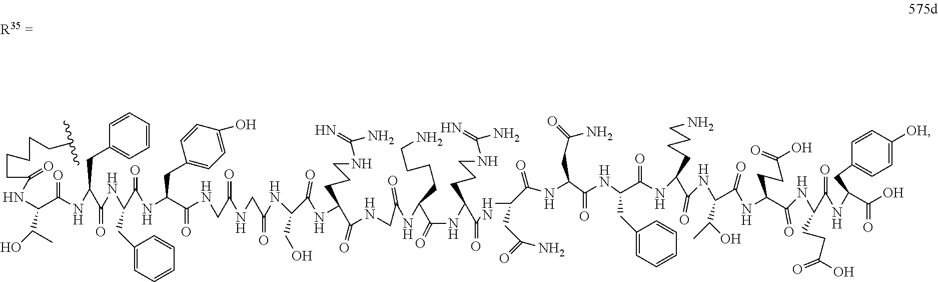 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

34.5 mg (56% yield), 94.3% pure, MS-ESI (m/z): [M+2H]²⁺+calcd for: C₁₆₈H₂₃₂FN₄1048, 1805.3478; found: 1805.3705;

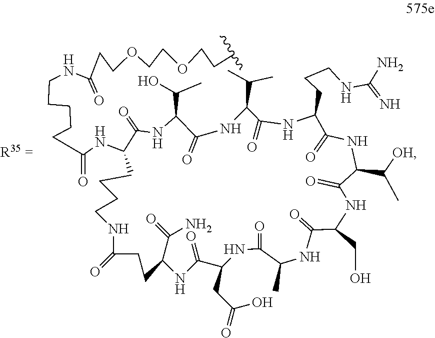 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

25 mg (59% yield), 96.1% pure by C18-HPLC, MS m/z, C₁₁₁H₁₆₅FN₂₇O₃₅ [M+H]⁺ 2455.1945; found, 2455.2206;

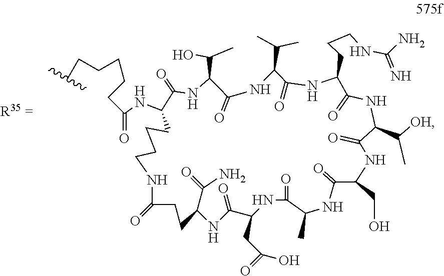 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

24 mg (610% yield), 96.2% pure by C18-HPLC, MS m/z, C₁₀₄H₁₅₂FN₂₆O₃₂ [M+H]⁺ 2296.1050; found, 2296.1288;

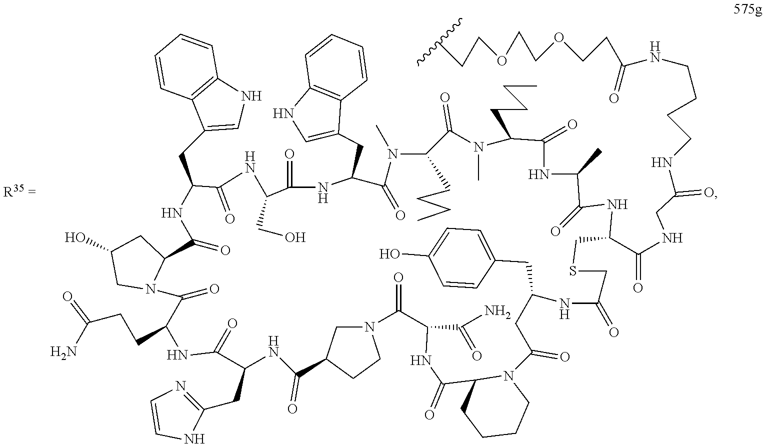 [SMILES: *CCOCCOCCC(=O)NCCCCNC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

31 mg (55% yield), 95.0% pure by C18-HPLC, MS m/z, C₁₅₈H₂₁₄FN₃₅O₄₀S [M+2H]²⁺ 1646.2746; found, 1646.2997;

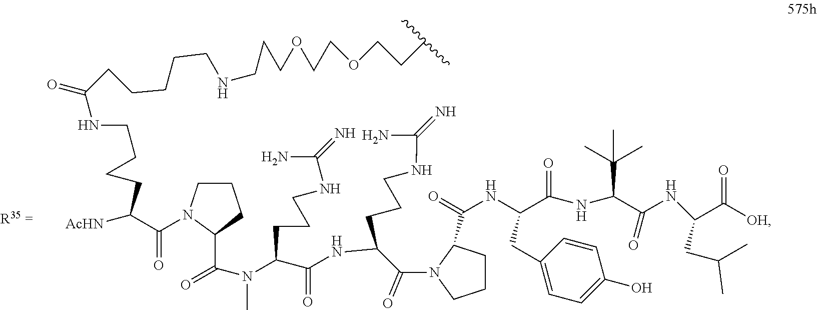 [SMILES: *CCOCCOCCCNCCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

26 mg (59% yield), 95.3% pure by C18-HPLC, MS m/z, C₁₂₃H₁₈4FN₂₈O₃₀ [M+H]⁺ 2552.3717; found, 2552.3985;

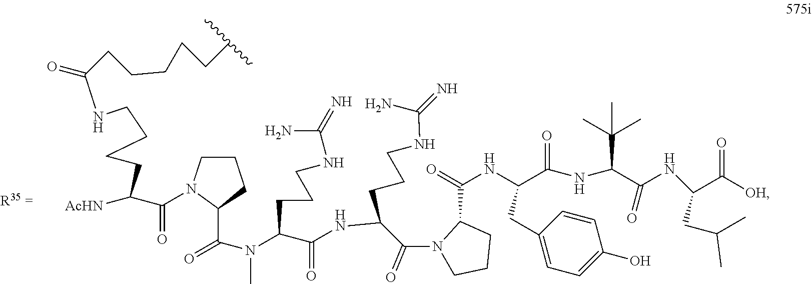

25 mg (60% yield), 95.4% pure by C18-HPLC, MS m/z, C₁₁₆H₁₆₉FN₂₇O₂₈ [M+H]⁺ 2407.2614; found, 2407.2878;

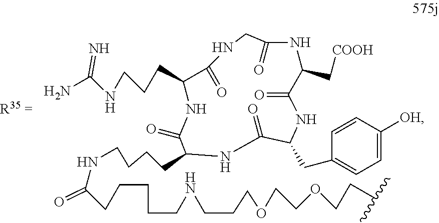 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

23 mg (70% yield), 95.2% pure by C18-HPLC, MS m/z, C₉₈H₁₃₈FN₂₂O₂₇ [M+H]⁺ 2074.0086; found, 2074.0315;

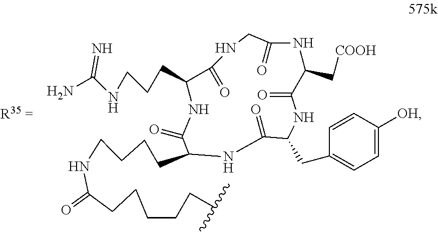 [SMILES: *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

20 mg (60% yield), 96.3% pure by C18-HPLC, MS m/z, C₉₁H₁₂₃FN₂₁O₂₅ [M+H]⁺ 1928.8983; found, 1928.9228.

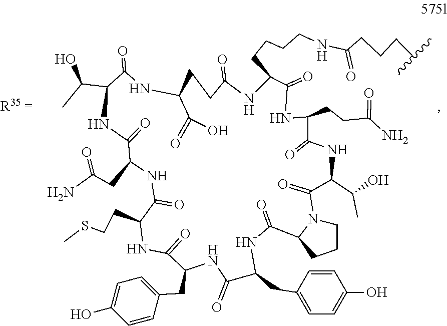 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

29 mg (71% yield), 95.2% pure by C18-HPLC, MS, C₁₁₈H₁₅₉FN₂₅O₃₅S [M+H]⁺ 2537.1135; found, 2537.1398.

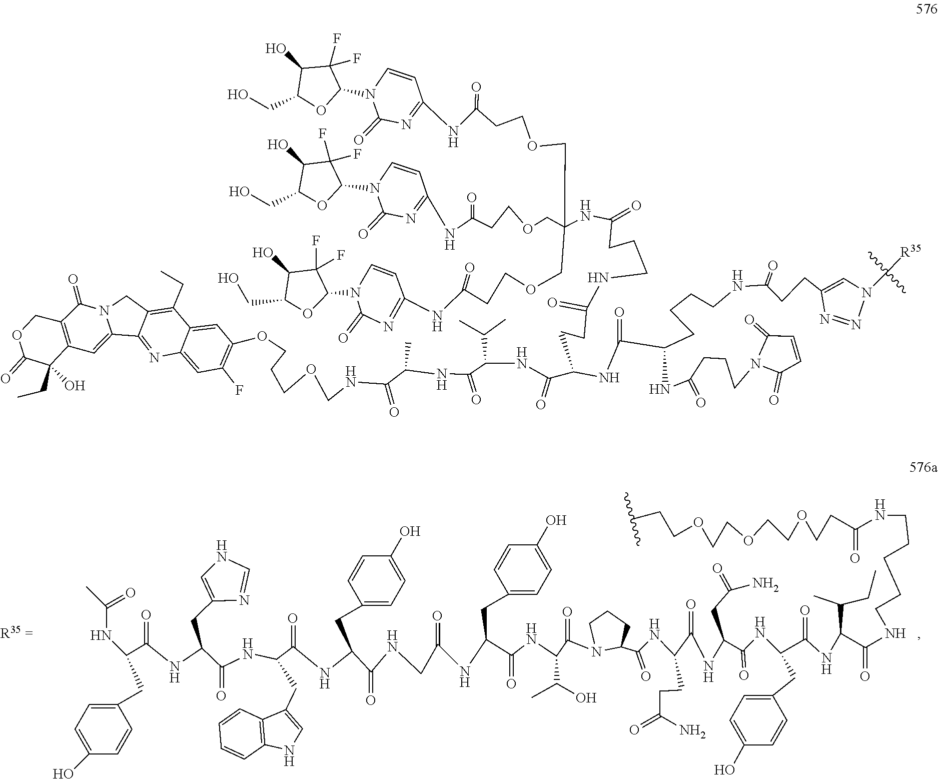

36 mg (49% o yield), 95.2% pure by HPLC, MS-ESI (m/z): [M+2H]²⁺ calcd for: C₁₉₇H₂₅₅F₇N₄₂O₅₈, 2134.9092; found: 2134.9385.

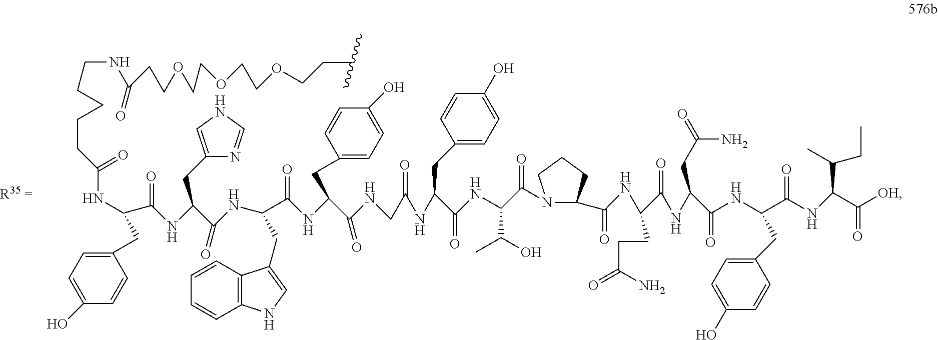 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

37 mg (51% yield), 95.3% pure by HPLC, MS-ES I (m/z): [M+2H]²⁺ calcd for: C₁₉₆H₂₅₂F₇N₄₁O₅₉, 2128.3933; found: 2128.4179.

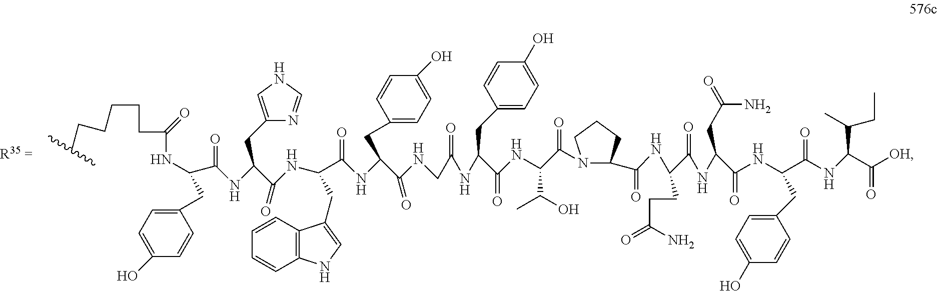 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

35 mg, (50% yield), 95.5% pure by HPLC, MS-ESI (m/z): [M+2H]²⁺ calcd for: C₈₇H₂₃₅F₇N₄₀O₅₅, 2026.8355; found: 2026.8626.

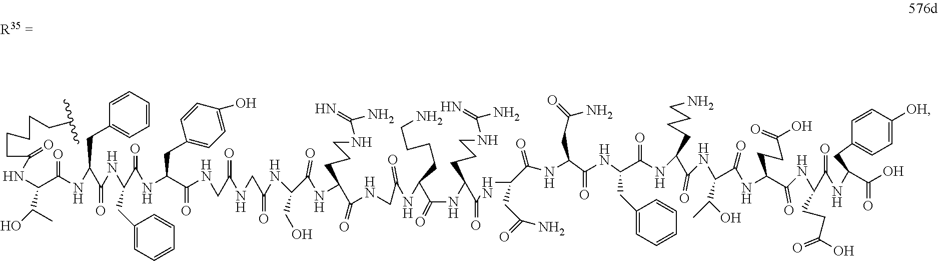 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

40.6 mg (50% yield), 94.7% pure, MS-ESI (m/z): [M+2H]²⁺+calcd for: C₂₁₂H₂₈₇F₇N₅₂O₅, 2375.0294; found: 2375.0557.

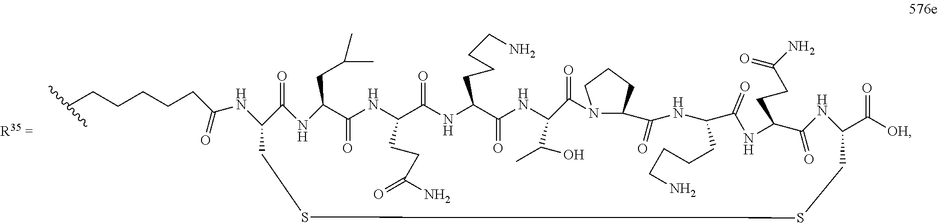 [SMILES: *CCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

35 mg(58% yield), 95.3% pure by C18-HPLC, MS m/z, C₁₅₁H₂₁₃F₇N₃₆O₄₈S₂[M+2H]²⁺ 1747.7331; found, 1747.7682;

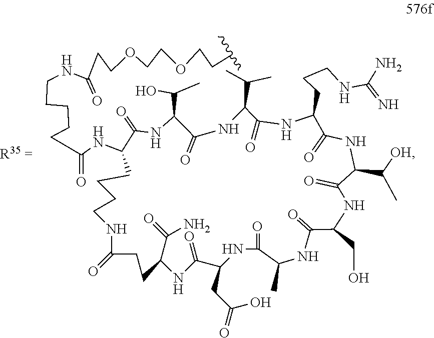 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

37 mg (57% yield), 96.4% pure by C18-HPLC, MS m/z, C₁₅₅H₂₂₁F₇N₃₈O₅₃ [M+2H]²⁺ 1797.7827; found, 1797.8095;

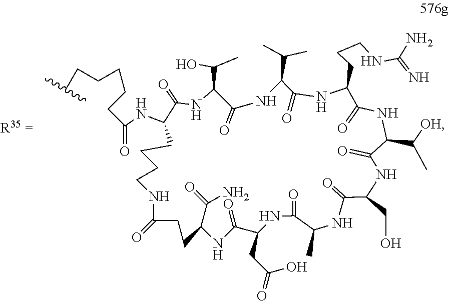 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

36 mg (61% yield), 96.8% pure by C18-HPLC, MS m/z, C₁₄₈H₂₀₈F₇N₃₇O₅₀ [M+2H]²⁺ 1718.2380; found, 1718.2615;

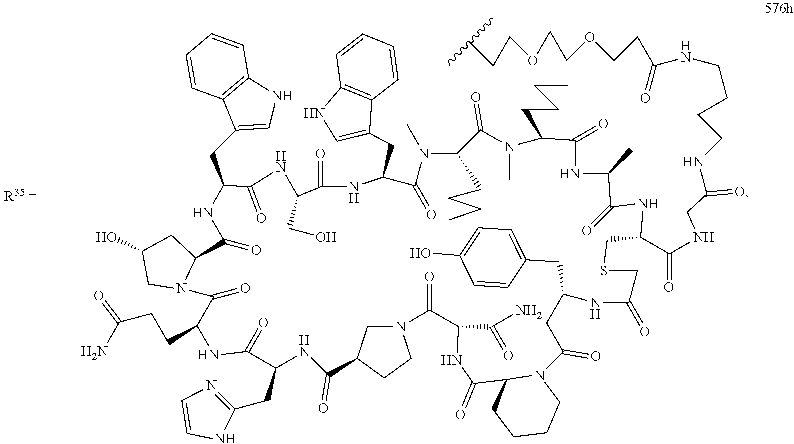 [SMILES: *CCOCCOCCC(=O)NCCCCNC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

42 mg (55% yield), 95.1% pure by C18-HPLC, MS m/z, C₂₀₂H₂₆₉F₇N₄₆O₅₁S₂ [M+2H]²⁺ 2215.9561; found, 2215.9812;

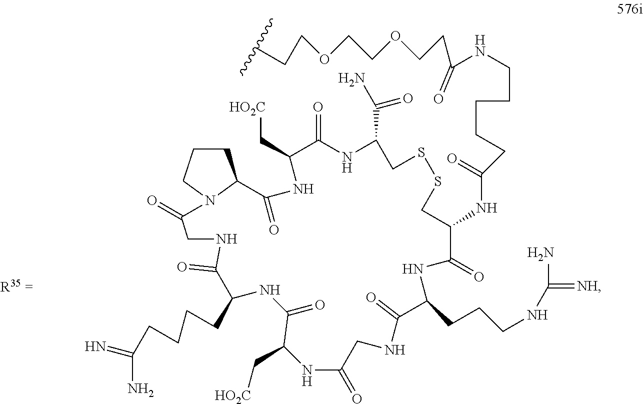 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCCC(=N)N)NC(=O)[C@H](CC(=O)O)NC(=O)CNC(=O)[C@H](CCCNC(=N)N)NC1=O]

35 mg (57% yield), 94.2% pure by C18-HPLC, MS, C₁₅₂H₂₀₉F₇N₄₀O₅₁S₂ [M+2H]²⁺ 1791.7160; found, 1791.7395;

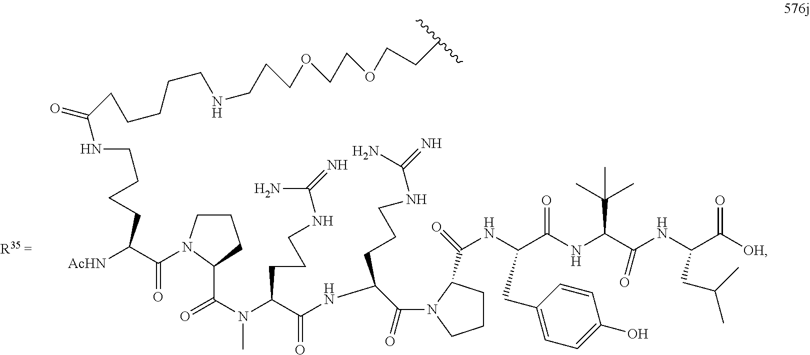 [SMILES: *CCOCCOCCCNCCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

35.7 mg (56% yield), 95.4% pure by C18-HPLC, MS m/z, C₁₆₇H₂₄₀F₇N₃₉O₄₈ [M+2H]²⁺ 1846.3713; found, 1846.3975;

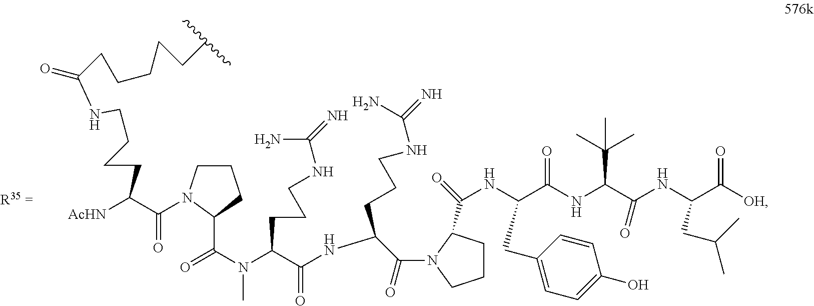 [SMILES: *CCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

37 mg (61% yield), 95.8% o pure by C18-HPLC, MS m/z, C₁₆₀H₂₂₈F₇N₃₈O₄₆ [M+2H]²⁺ 1773.8161; found, 1773.8398;

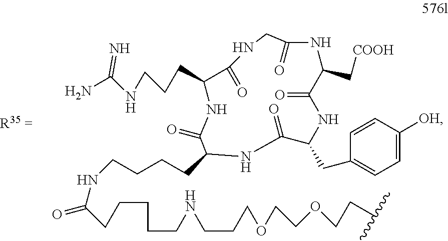 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

33 mg (60% yield), 95.5% pure by C18-HPLC, MS m/z, C₁₂H₁₉₄F₇N₃₃O₄ₛ[M+2H]²⁺ 07.1897; found, 1607.2129;

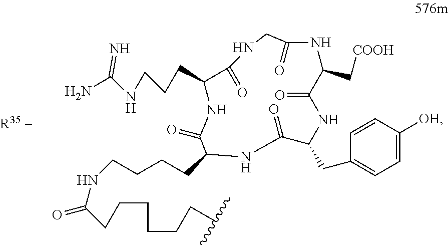 [SMILES: *CCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

31 mg (570% yield), 96.4% pure by C18-HPLC, MS m/z, C₁₃₅H₁₉F₇N₃₂O₄₃ [M+2H]²⁺ 1577.1791; found, 1577.2016;

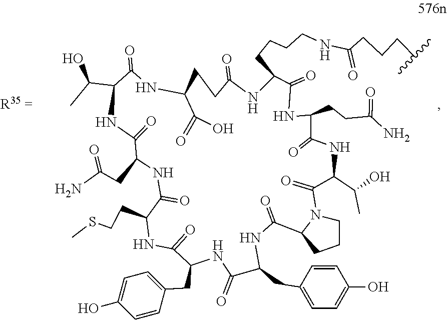 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

40 mg (64% yield), 95.4% pure by C18-HPLC, MS C₁₆₂H₂₁₅F₇N₃₆O₅₃S [M+2H]²⁺ 1838.7422; found, 1838.7678.

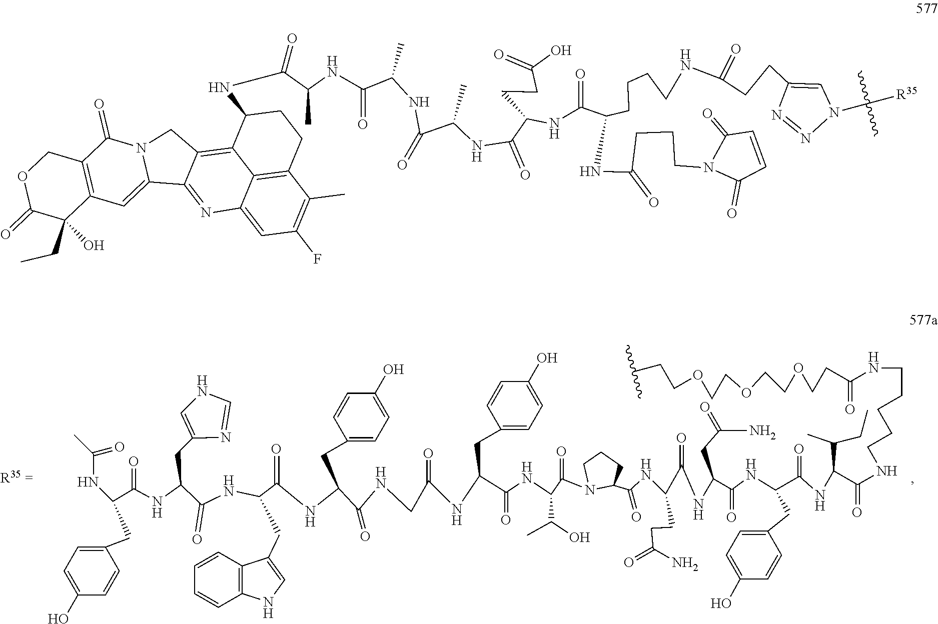

33 mg (62o yield), 95.3% pure by HPLC, MS-ESI (m/z): [M+2H]²⁺ calcd for: C₁₅2H₁₉₅FN₃₂O₃₉, 1555.7121; found: 1555.7369.

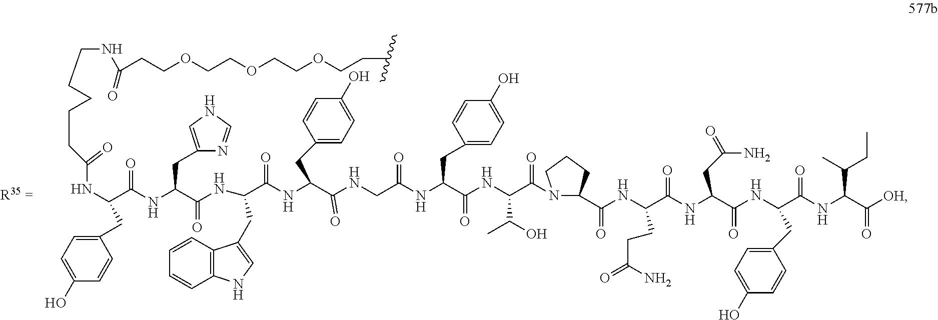 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

34 mg (64% yield), 95.6% pure by HPLC, MS-ESI (m/z): [M+2H]²⁺ calcd for: C₁₅₁H₁₉₂FN₃₁O₄₀, 1549.1963; found: 1549.2197.

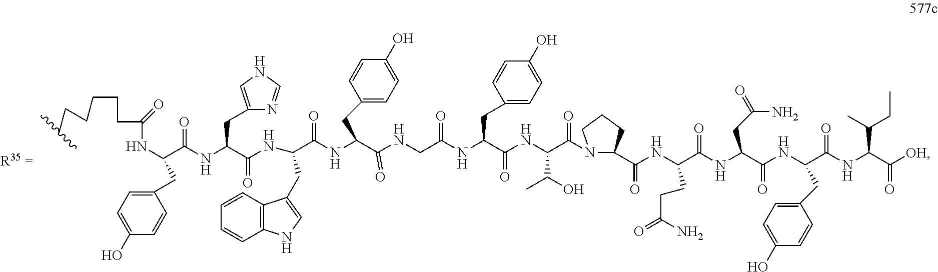 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

35 mg, (70% yield), 95.9% pure by HPLC, MS-ESI (m/z): [M+2H]²⁺ calcd for: C₁₃₇H₁₆₇FN₂₉O₃₃, 1447.6384; found: 1447.6615.

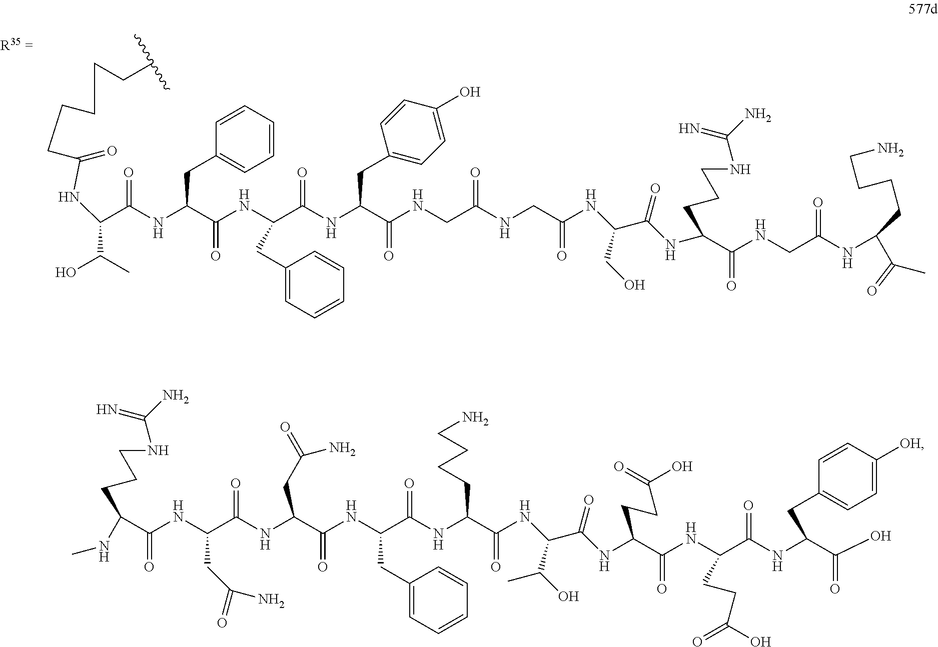

35.6 mg (58% yield), 94.4% pure, MS-ESI (m/z): [M+2H]²⁺ calcd for: C 167H₂₂₇FN₄₂O₄₇, 1795.8324; found: 1795.8585.

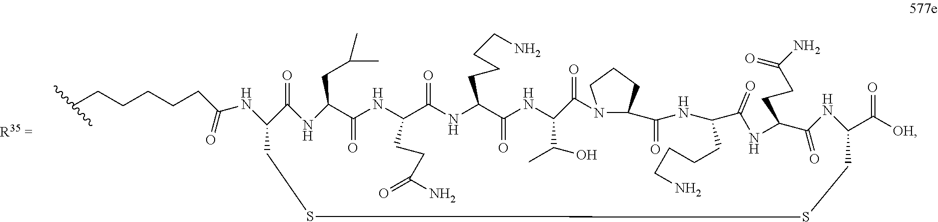 [SMILES: *CCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

28 mg (70% yield), 95.2% pure by C18-HPLC, MS m/z, C₁₀₆H₁₅₂FN₂₆O₂₉S₂[M+H]⁺ 2336.0644; found, 2336.0898;

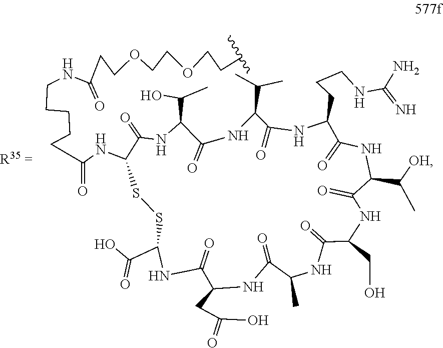 [SMILES: *CCOCCOCCC(=O)NCCCCCC(=O)N[C@H]1SS[C@@H](C(=O)O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

24 mg (59% yield), 94.8% pure by C18-HPLC, MS, C₁₀₃H₁₄₆FN₂₆O₃₄S₂[M+H]⁺ 2373.9920 found, 2374.0186;

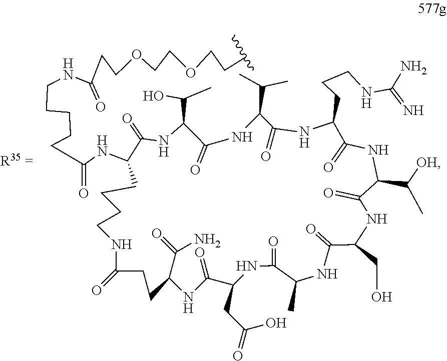 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

27 mg (64% yield), 96.3% pure by C18-HPLC, MS m/z, C₁₁₀H₁₆₀FN₂₈O₃₄ [M+H]⁺ 2436.1636; found, 2436.1881;

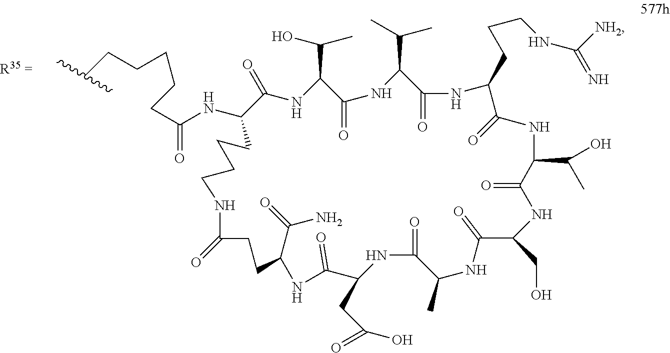 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

27 mg (69% yield), 96.4% pure by C18-HPLC, MS m/z, C₁₀₃H₁₄₇FN₂₇O₃₁ [M+H]⁺ 2277.0740; found, 2277.0998;

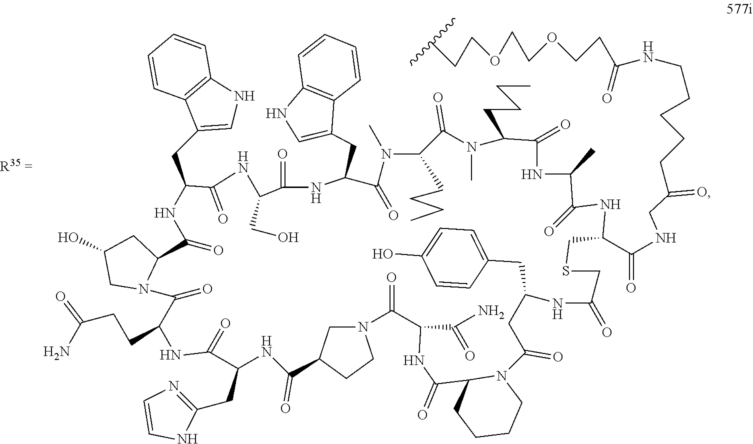 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

33 mg (58% yield), 95.3% pure by C18-HPLC, MS m/z, C₁₅₇H₂₀₉FN₃₆O₃₉S [M+2H]²⁺ 1636.7591; found, 1636.7855;

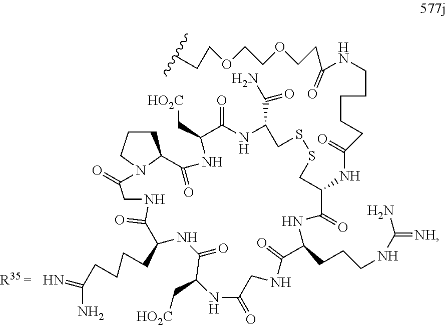 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCCC(=N)N)NC(=O)[C@H](CC(=O)O)NC(=O)CNC(=O)[C@H](CCCNC(=N)N)NC1=O]

26 mg (62% yield), 94.7% pure by C18-HPLC, MS, C₁₀₅H₁₄₈FN₃₀O₃₂S₂[M+H]⁺ 2424.0301; found, 2424.0563;

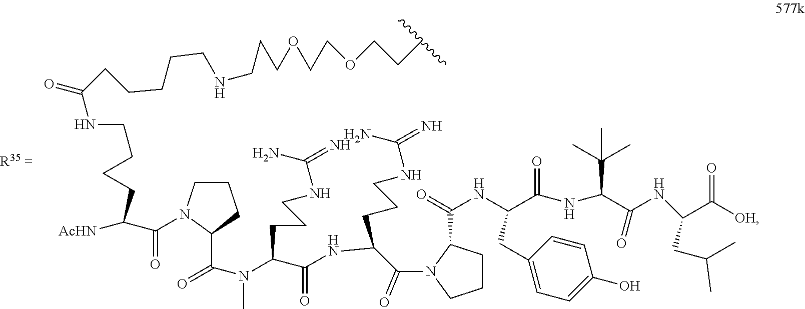 [SMILES: *CCOCCOCCCNCCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

27 mg (62% yield), 95.4% pure by C18-HPLC, MS m/z, C₁₂₂H₁₇₉FN₂₉O₂₉ [M+H]⁺ 2533.3408; found, 2533.3676;

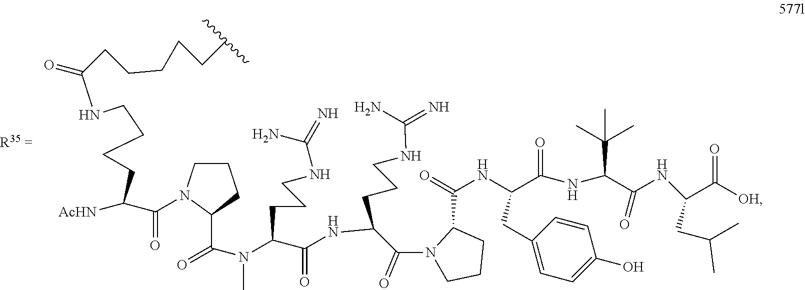 [SMILES: *CCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

27 mg (70% yield), 95.7% pure by C18-HPLC, MS m/z, C₁₁₅H₁₆₄FN₂₈O₂₇ [M+H]⁺ 2388.2305; found, 2388.2569;

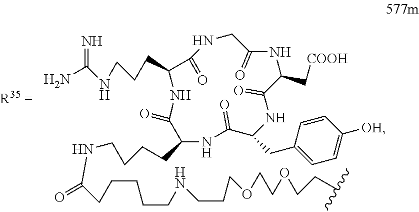 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

23 mg (65% yield), 95.3% pure by C18-HPLC, MS m/z, C₉₇H₁₃₃FN₂₃O₂₆ [M+H]⁺ 2054.9776; found, 2055.0045;

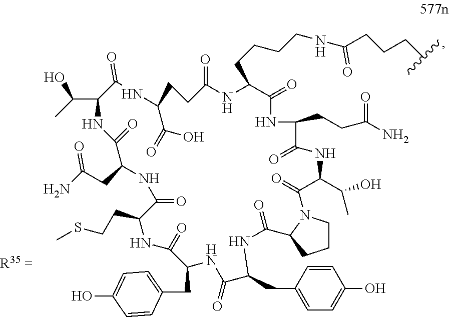 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

30 mg (69% yield), 95.1% pure by C18-HPLC, MS, C₁₁₇H₁₅₄FN₂₆O₃₄S [M+H]⁺ 2518.0826; found, 2518.1073.

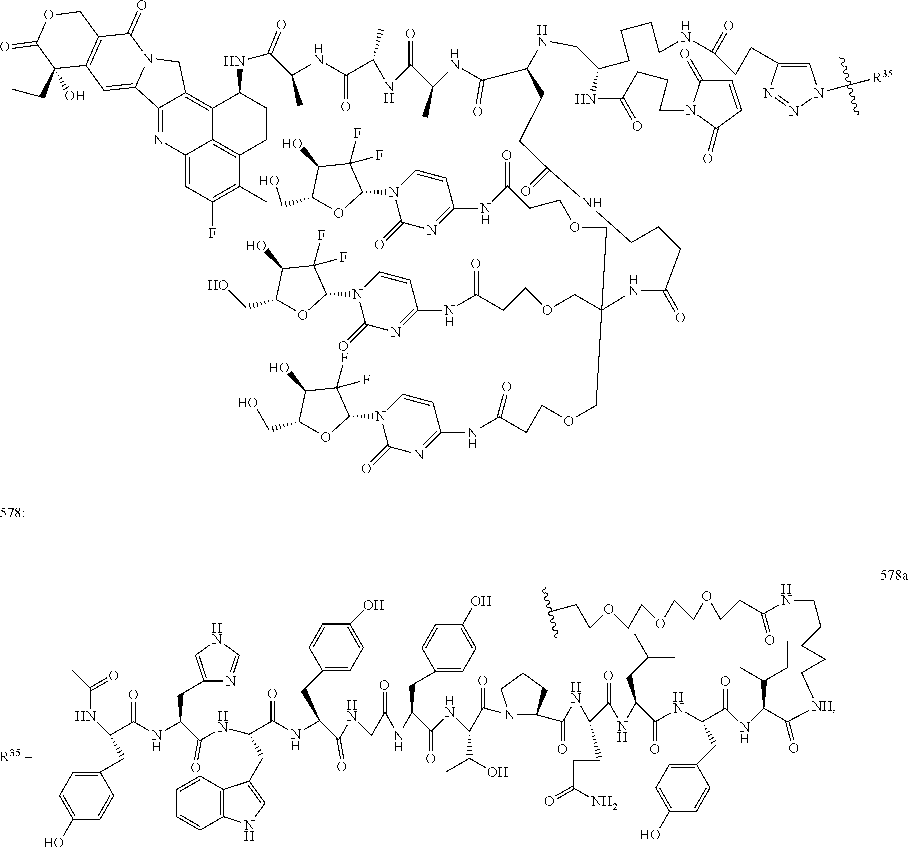

36 mg (53% yield), 95.4% pure by HPLC, MS-ESI (m/z): [M+2H]²⁺ calcd for: C₁₉₆H₂₅₀F₇N₄₃O₅₇, 2125.3936; found: 2125.4162;

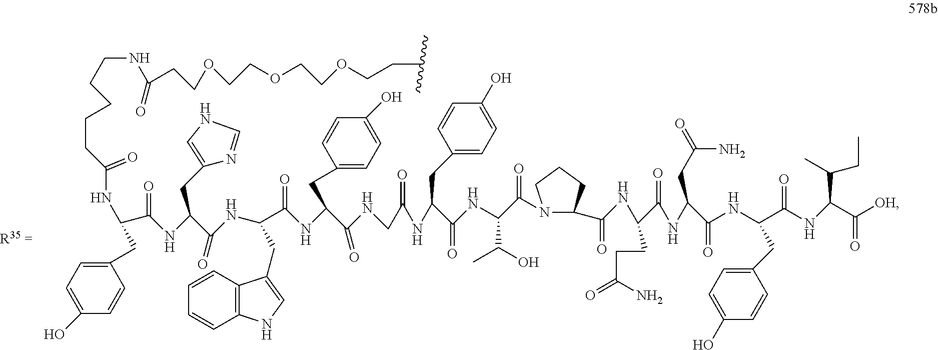 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

38 mg (52% yield), 95.6% pure by HPLC, MS-ESI (m/z): [M+2H]²⁺ calcd for: C₁₉₅H₂₄₇F₇N₄₂O₅₈, 2118.8779; found: 2118.9009;

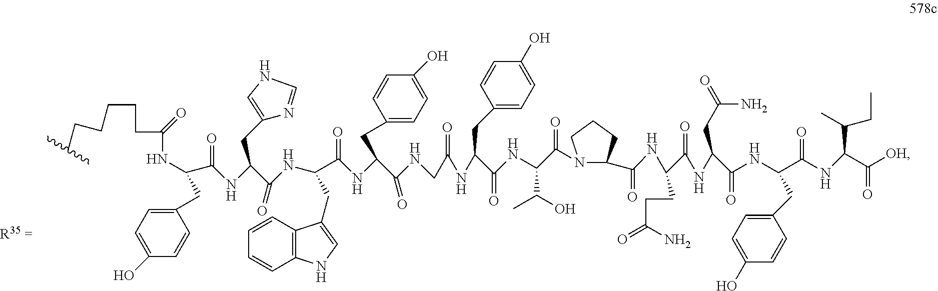 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

35 mg, (50% yield), 96.1% pure by HPLC, MS-ESI (m/z): [M+2H]²⁺ calcd for: C₁₈₆H₂₃₀F₇N₄₁O₅₄, 2017.3199; found: 2017.3435;

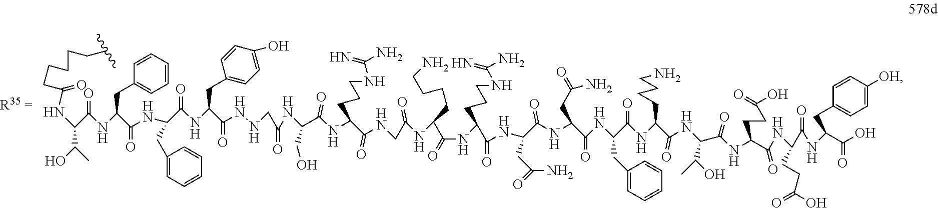 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NNCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

42 mg (51% yield), 94.9% pure, MS-ESI (m/z): [M+2H]²⁺+calcd for: C₂₁₁H₂₈₂F₇N₅₃O₆₅, 2365.5138; found: 2365.5368;

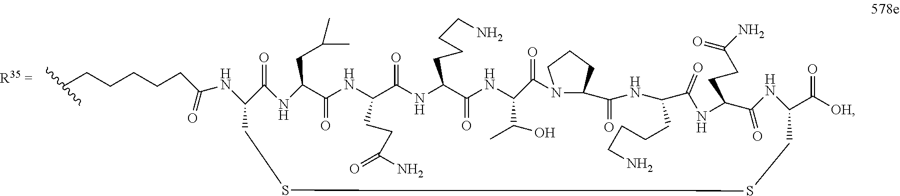 [SMILES: *CCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

35 mg (58% yield), 95.1% pure by C18-HPLC, MS m/z, C₁₅₀H₂₀₈F₇N₃₇O₄₇S₂[M+2H]²⁺ 1738.2175; found, 1738.2397;

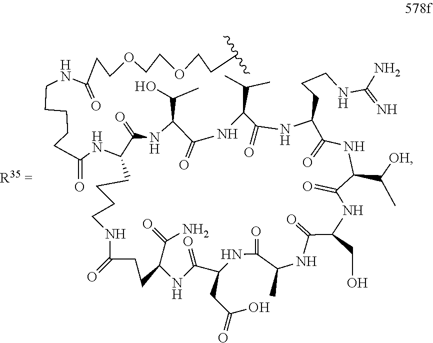

37 mg (60% yield), 96.2% pure by C18-HPLC, MS m/z, C₁₅₄H₂₁₆F₇N₃₉O₅₂ [M+2H]²⁺ 1788.2671; found, 1788.2908;

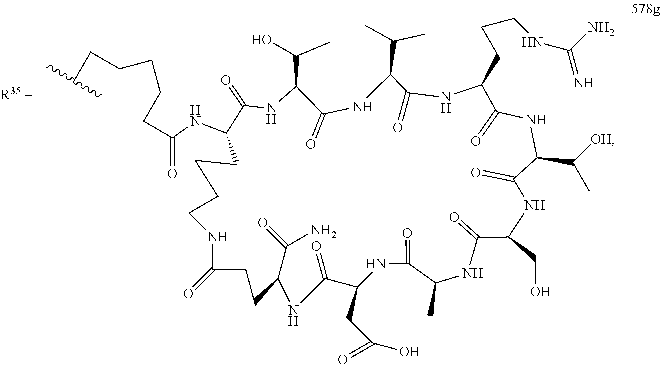 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

36 mg (61% yield), 96.7% pure by C18-HPLC, MS m/z, C₁₄₇H₂₀₃F₇N₃₈O₅₉ [M+2H]²⁺ 1708.7224; found, 1708.7369;

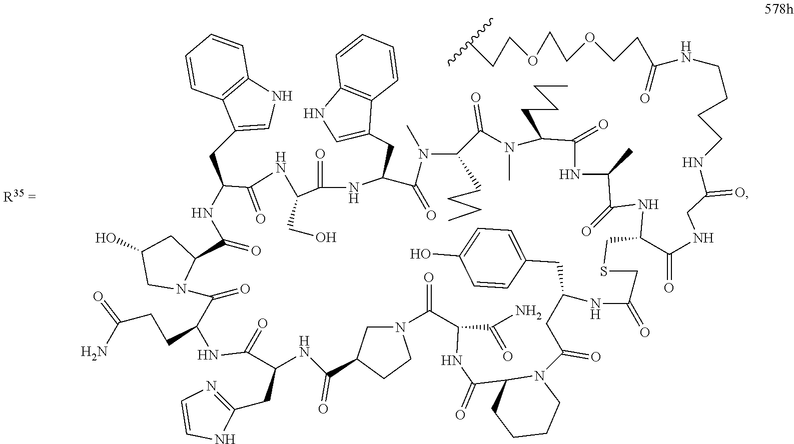 [SMILES: *CCOCCOCCC(=O)NCCCCNC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

41 mg (54o yield), 95.0% pure by C18-HPLC, MS m/z, C₂a1H₂₆₄F₇N₄₇O₅₇S [M+2H]²⁺ 2206.4406; found, 2206.4636;

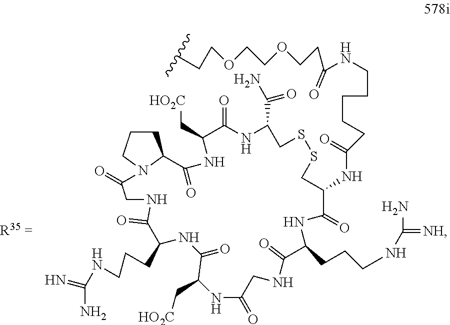 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

35 mg (57% yield), 94.7% pure by C18-HPLC, MS, C₁₄₉H₂₀4F₇N₄₁O₅₀S₂ [M+2H]²⁺ 1782.2004; found, 1783.2249;

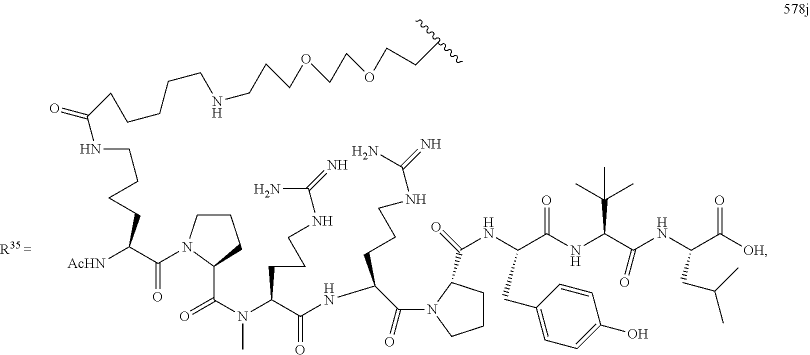

36 mg (57% yield), 95.5% pure by C18-HPLC, MS m/z, C₁₆₆H₂₃₃F₇N₄₀O₄₇ [M+2H]²⁺ 1836.8557; found, 1837.8792;

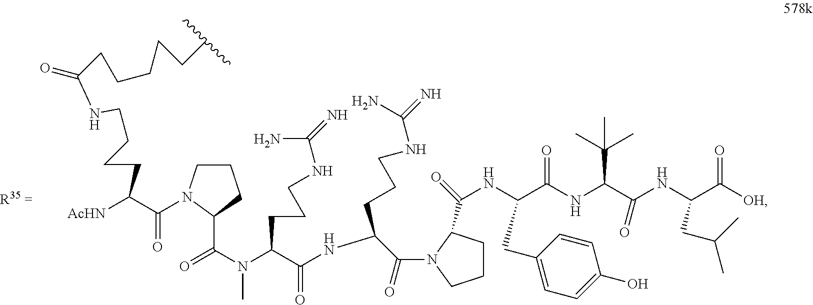

37 mg (61% yield), 95.8% pure by C18-HPLC, MS m/z, C₁₅₉H₂₂₂F₇N₃₉O₄₅ [M+2H]²⁺ 1764.3006; found, 1764.3255;

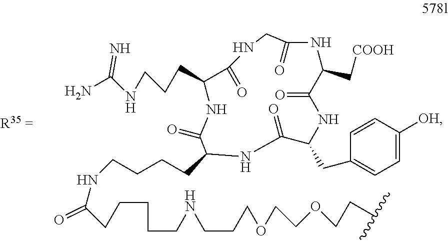 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

33.5 mg (61% yield), 95.6% pure by C18-HPLC, MS m/z, C₁₄₁H₁₉₁F₇N₃₄O₄₄ [M+2H]²⁺ 1597.6742; found, 1597.6986;

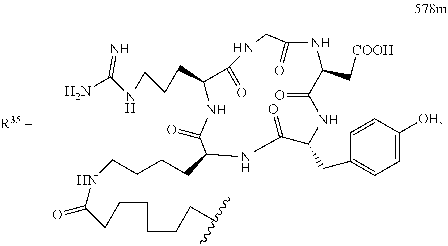 [SMILES: *CCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

31 mg (59% yield), 96.4% pure by C18-HPLC, MS m/z, C₁₃₄H₁₇₂F₇N₃₃O₄₂ [M+2H]²⁺ 1525.1190; found, 1525.1413;

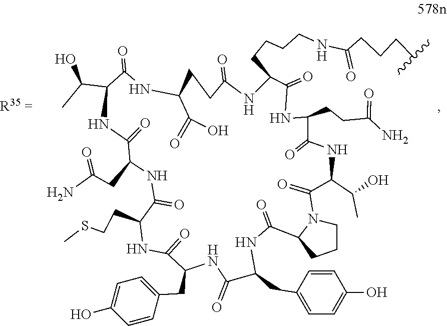 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

41 mg (65% yield), 95.5% pure by C18-HPLC, MS, C₁₆₁H₂₁₀F₇N₃₇O₅₂S [M+2H]²⁺ 1829.2266; found, 1829.2598.

### Example 398. General synthesis of CPT/linker complexes containing dual ligand compounds via a click chemistry

In a glass tube with a screw cap, compound 521 (40 mg, 0.0171 mmol, 1.0 equiv), or 523 (40 mg, 0.0174 mmol, 1.02 equiv), or 563 (89 mg, 0.0172 mmol, 1.01 eq), or 568 (90 mg, 0.0173 mmol, 1.01 eq), and a ligand (compound 470~498) containing azido terminal (2.0~2.2 equiv) dissolved in DMA (5 ml) was added sodium ascorbate (250 mM in H₂O, 0.680 ml, 0.170 mmol, 10.0 equiv). After vortexed for a few seconds, a solution containing CuSO₄ and BTTA (2-[4-{(bis[(1-tert-butyl-1H-1,2,3-triazol-4-yl)methyl]amino)-methyl}-1H-1,2,3-triazol-1-yl]-acetic acid) (DMA-H₂O 1:10, 10 mM CuSO₄, 11 mM BTTA, 3.420 ml, 2.0 equiv CuSO₄, 2.2 equiv BTTA) was added in, and the reaction mixture was vortexed and agitated on an orbital shaker at 25° C. for 18 h. After that, EDTA (200 mM, 0.300 ml, 0.600 mmol) was added, the mixture was vortexed, heated over hot water bath (80° C.) without the screw cap for 2 h for removal of the protected furan, concentrated in a rotary evaporator connected with a vacuum pump, and then purified by C18 prep-HPLC (20 mm×250 mm), eluted with water/CH₃CN from 25% CH₃CN to 90% CH₃CN in 50 min, 8 ml/min. The fractions containing the products were pooled, evaporated, and lyophilized to afford the planned product (35%~75% yield).

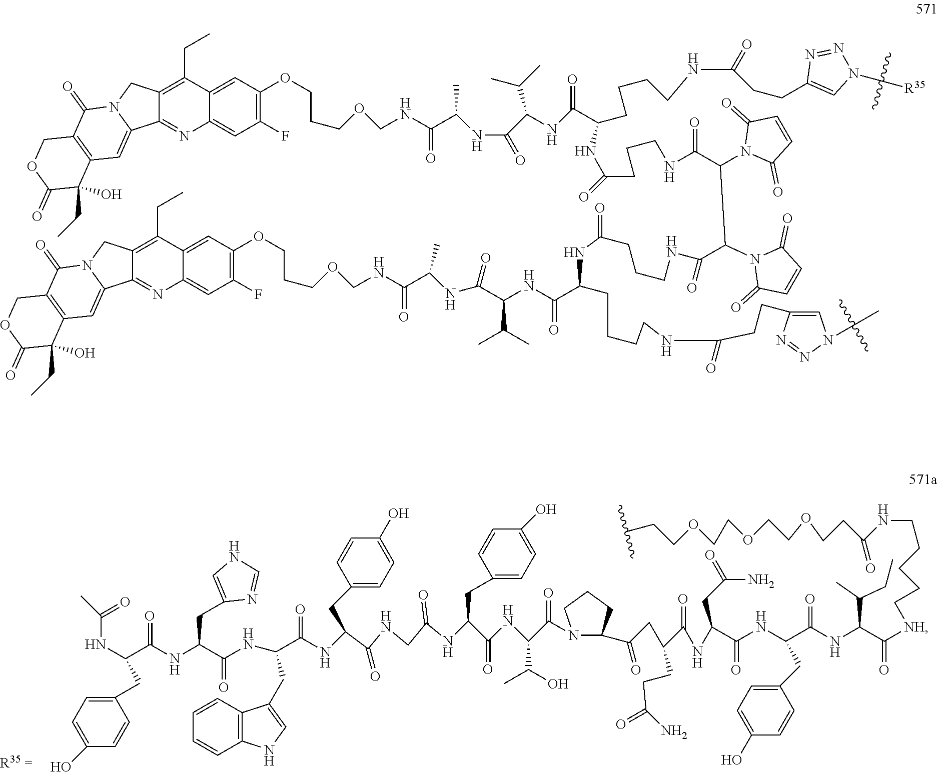

44 mg (42% yield), 95.2% pure by HPLC, MS-ESI (m/z): [M+3H]3+calcd for: C₃₀₀H₃₉₀F₂N₆₂O₇₆, 2037.9483; found: 2037.9630.

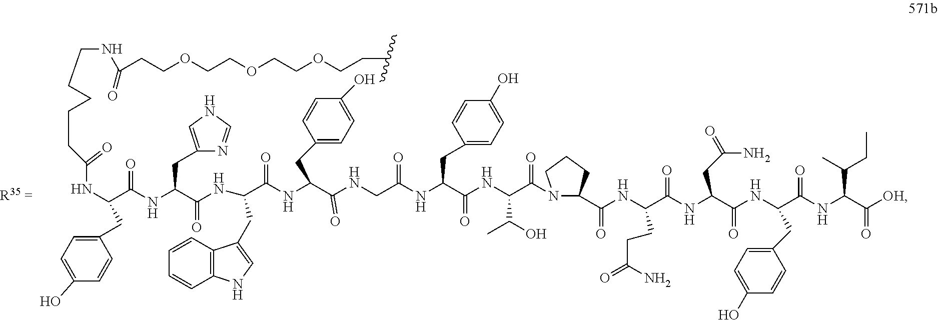 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

41.5 mg (40% yield), 95% pure by HPLC, MS-ESI (m/z): [M+3H]³⁺ calcd for: C₂₉₈H₃₈₁F₂N₆₀O₇₈, 2029.2605; found: 2029.2755.

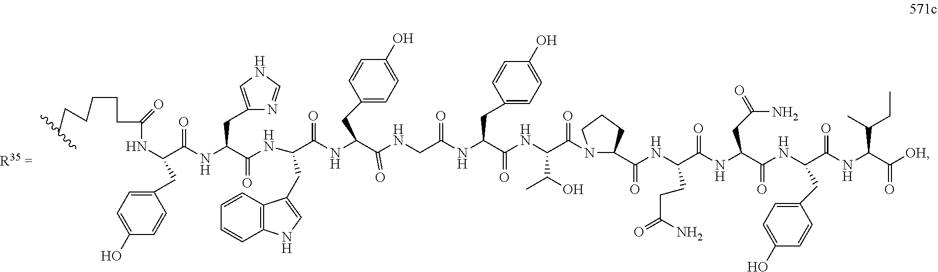 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

44.2 mg, (44% yield), 95% pure by HPLC, MS-ESI (m/z): [M+3H]3+calcd for: C₂₈₀H₃₄₉F₂N₅₈O₇₀, 1893.8500; found: 1893.8537.

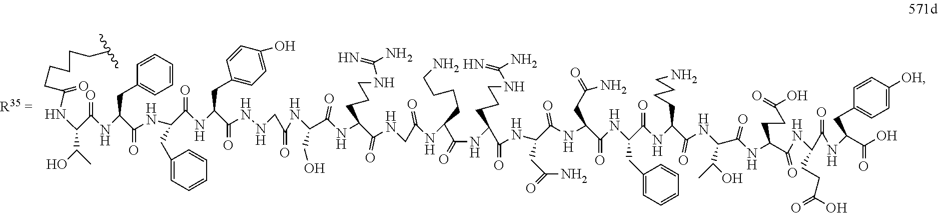 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NNCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

46 mg (38% yield), 94.2% pure, MS-ESI (m/z): [M+3H]³⁺ calcd for: C₃₃₀H₄₅₃F₂N₈₂O₉₂, 2358.1086; found: 2358.1308.

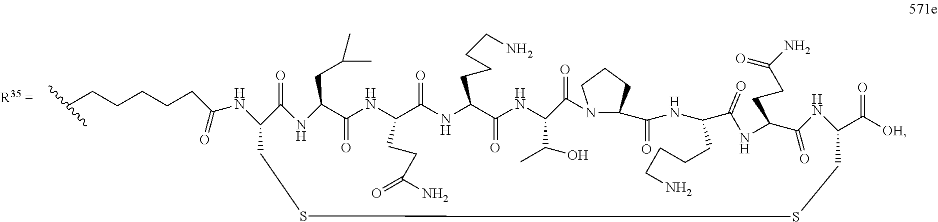 [SMILES: *CCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

30.5 mg (39% yield), 94.8% pure by C18-HPLC, MS, C₂₀₈H₃₀₃F₂N₅₀O₅₆S₄[M+2H]²⁺ 2282,0664; found, 2282.0825;

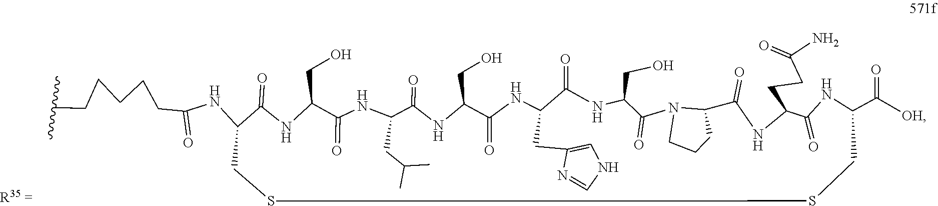

30 mg(39% yield), 95.1% pure by C18-HPLC, MS, C₁₉₆H₂₆₉F₂N₄₈O₅₈S₄ [M+2H]²⁺

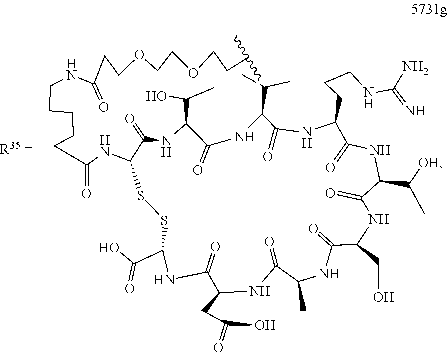

32 mg (41% yield), 95.0% pure by C18-HPLC, MS, C₂₀₂H₂₉F₂N₅₀O₆₆S₂[M+2H]²⁺ 2319.9940; found, 2320.0205;

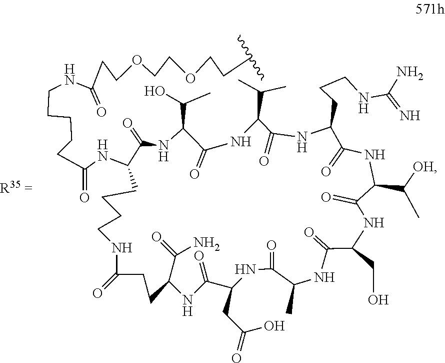

35 mg (43% yield), 96.20% pure by C18-HPLC, MS, C₂₁₆H₃₁₉F₂N₅₄O₆₆ [M+2H]²⁺ 2382.1656; found, 2382.1908;

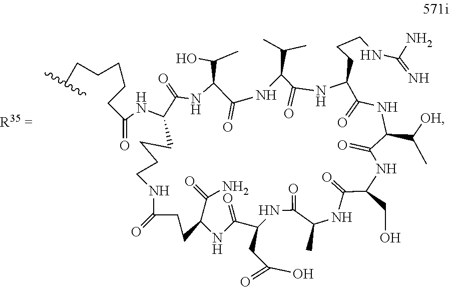 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

36 mg (48% yield), 96.5% pure by C18-HPLC, MS, C₂₀₂H₂₉₃F₂N₅₂O₆₀ [M+2H]²⁺ 2223.0761; found, 2223.0995;

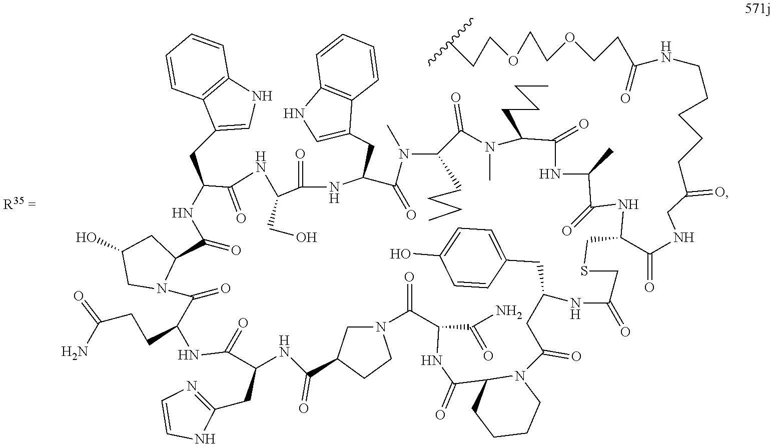 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

27 mg (3% yield), 9.% pure by C18-HPLC, MS m/z, C₃₁₀H₄₁₈F₂N₇₀O₇₆S₂[M+3H]³⁺ 2146.0109; found, 2146.0295;

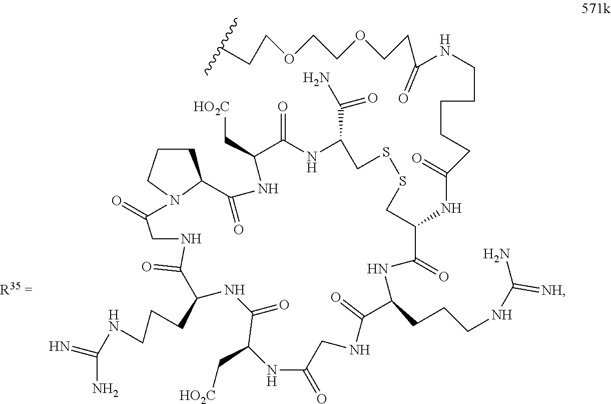 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

36 mg (45 yield), 95.1% pure by C18-HPLC, MS, C₂₀CH₂₉₆F₂N₅₈O₆₂S₄[M+2H]²⁺ 2370.0321; found, 2370.0605;

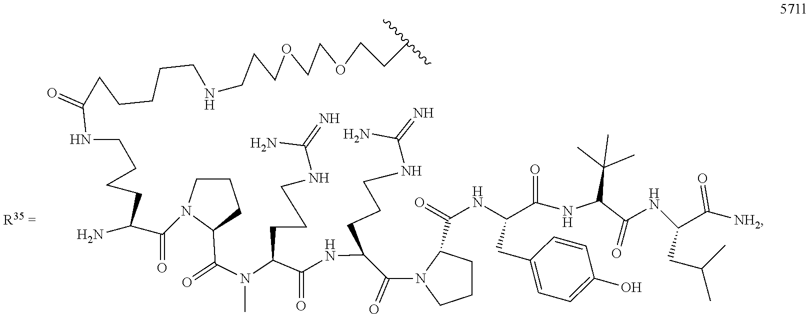

35.8 mg (43% yield), 96.5% pure by C18-HPLC, MS m/z, C₂₃₆H₃₅₆F₂N₅₈O₅₂ [M+2H]²⁺ 2436.3482; found, 2436.3695;

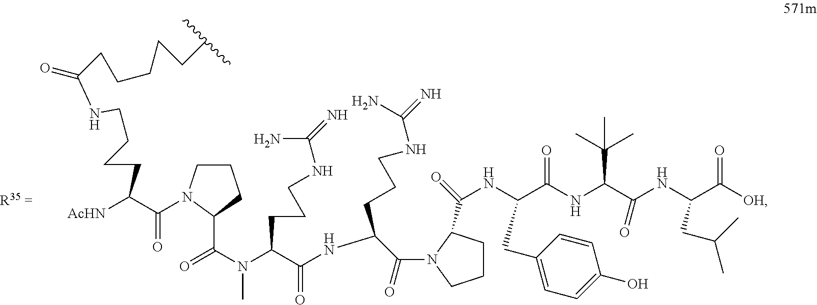

35 mg (44% yield), 96.7% pure by C18-HPLC, MS m/z, C₂₂₆H₃₃₀F₂N₅₆₅₀ [M+2H]²⁺ 2333.2484; found, 2333.2484;

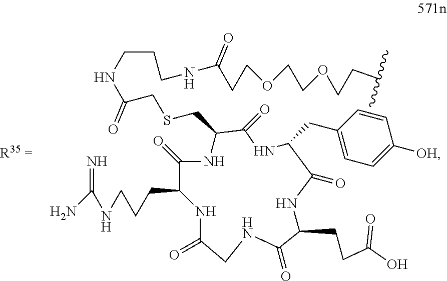 [SMILES: *CCOCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

31 mg (45% yield), 96.1% pure by C18-HPLC, MS m/z, C₁₈₄H₂₅₀F₂N₄₄O₅₂S₂[M+2H]²⁺ 2004.8840; found, 2004.9025;

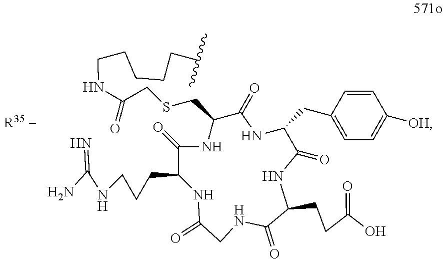 [SMILES: *CCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

0 29 mg (45% yield), 96.0% pure by C18-HPLC, MS m/z, C₁₇₄H₂₃₂F₂N₄₂O₄₆S₂[M+2H]²⁺ 1873.8257; found, 1873.8405;

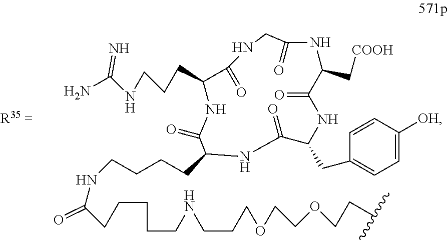 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

35 mg (51% yield), 95.8% pure by C18-HPLC, MS m/z, C₁₉₀H₂₆₆F₂N₄₄O₅₀ [M+2H]²⁺ 2000.9796; found, 2000.9995;

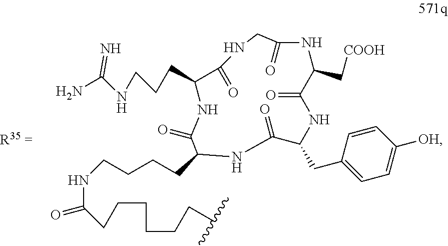 [SMILES: *CCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

36 mg (56% yield), 96.8% pure by C18-HPLC, MS m/z, C₁₇₆H₂₃₆F₂N₄₂O₄₆ [M+2H]²⁺ 1855.8693; found, 1855.8915.

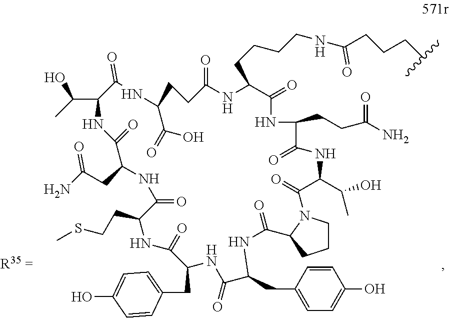 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

44.6 mg (56% yield), 95.1% pure by C18-HPLC, MS, C₂₃₀H₃₀₈F₂N₅₀O₆₆S₂[M+2H]²⁺ 2464.0845; found, 2464.1038.

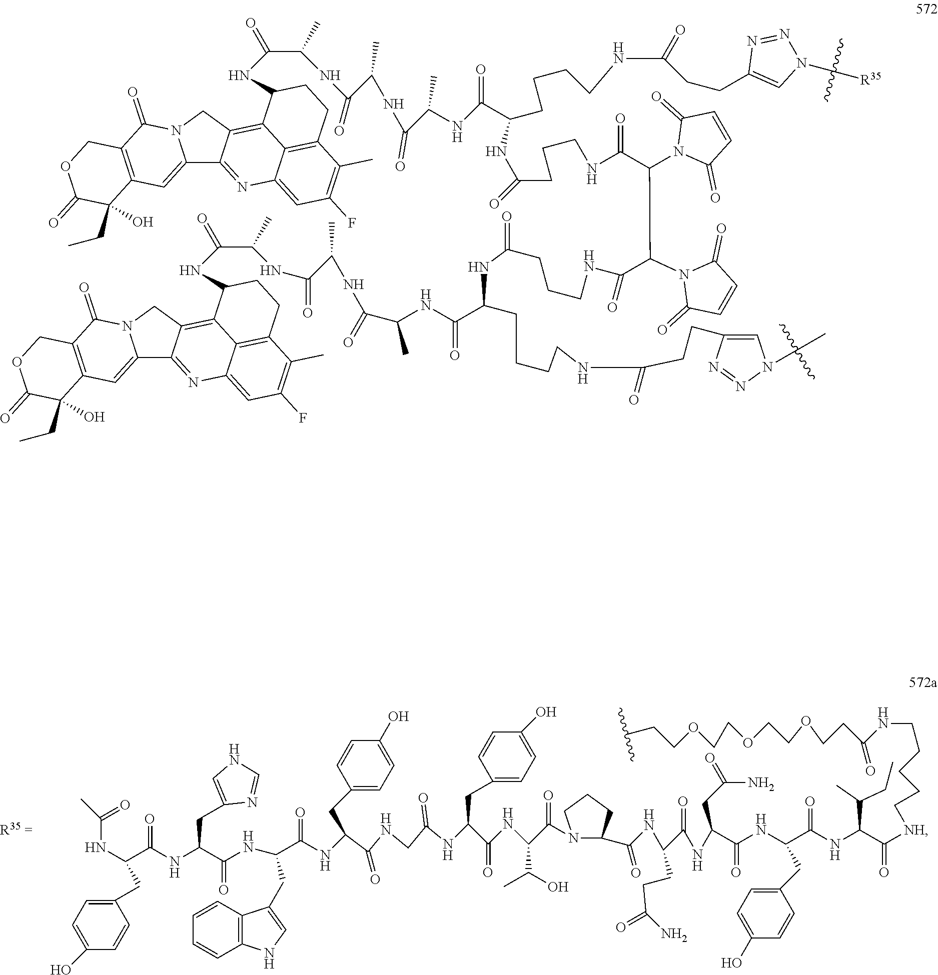

46 mg (44% yield), 95.4% pure by HPLC, MS-ESI (m/z): [M+3H]³⁰ calcd for: C₂₉₈H₃₇₉F₂N₆₄O₇₄, 2025.2610; found: 2025.2935.

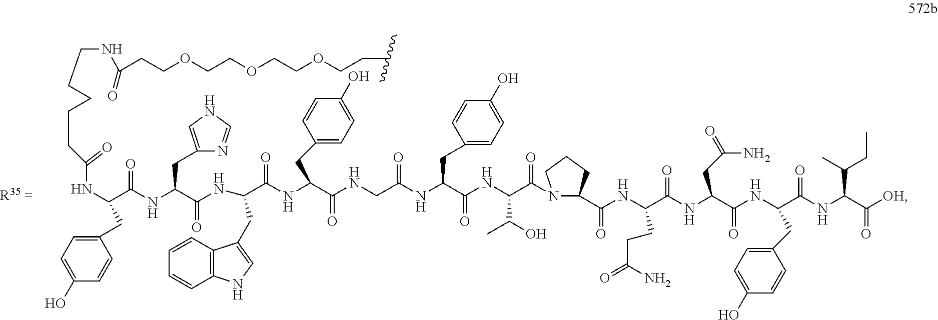 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

43.5 mg (42% yield), 95.1% pure by HPLC, MS-ESI (m/z): [M+3H]³⁺ calcd for: C₂₉₆H₃₇₃F₂N₆₂O₇₆, 2016.5732; found: 2016.5993.

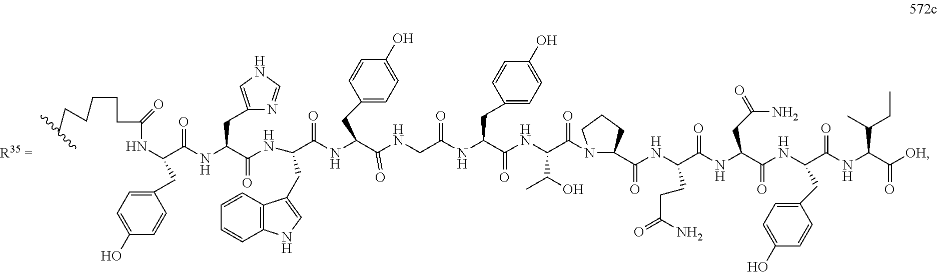 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

41 mg, (43% yield), 95.8% pure by HPLC, MS-ESI (m/z): [M+3H]³⁺ calcd for: C₂₇₈H₃₃₉F₂N₆₀O₆₈, 1881.1627; found: 1881.1853.

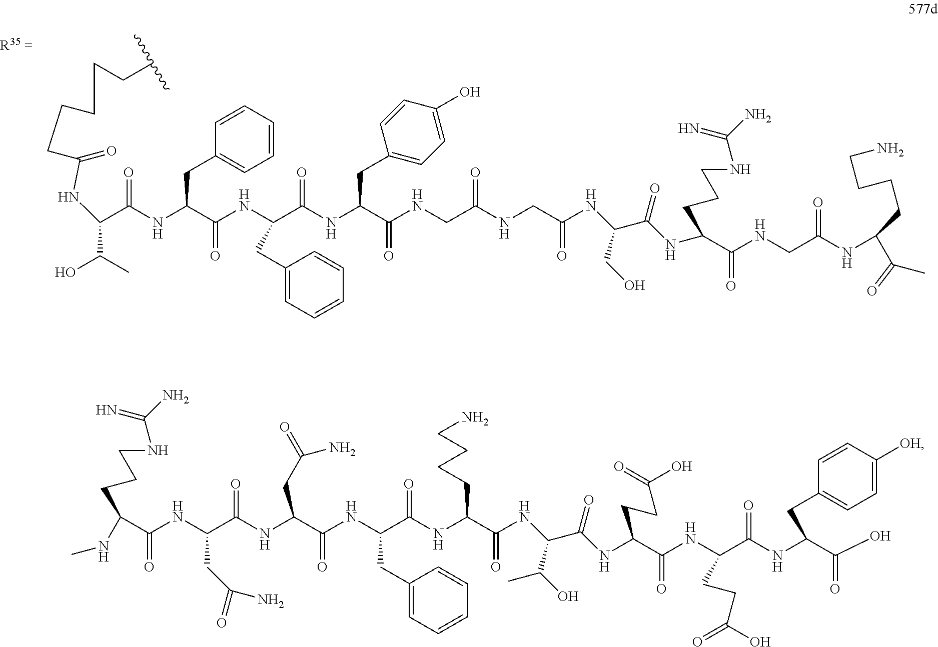

47
mg (39% yield), 94.7% pure, MS-ESI (m/z): [M+3H]³⁺ calcd for: C₃₂₈H₄₄₃F₂N₈₄O₉₀, 2345.4213; found: 2345.4547.

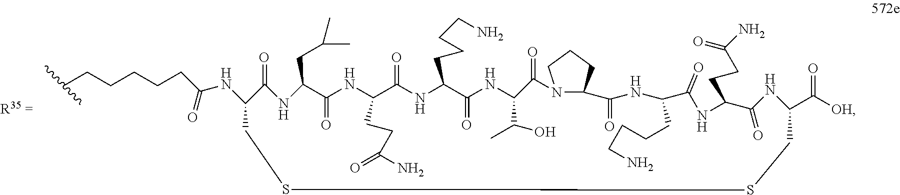 [SMILES: *CCCCCC(=O)N[C@H]1CSSC[C@@H](C(=O)O)NC(=O)[C@H](CCC(N)=O)NC(=O)[C@H](CCCCN)NC(=O)[C@@H]2CCCN2C(=O)[C@H](C(C)O)NC(=O)[C@H](CCCCN)NC(=O)[C@H](CCC(N)=O)NC(=O)[C@H](CC(C)C)NC1=O]

32 mg (40% yield), 94.8% pure by C18-HPLC, MS, C₂₀₆H₂₉₄F₂N₅₂O₅₄S₄[M+2H]²⁺ 2263.0354; found, 2263.0586;

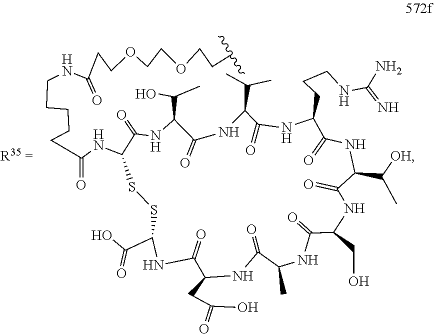 [SMILES: *CCOCCOCCC(=O)NCCCCCC(=O)N[C@H]1SS[C@@H](C(=O)O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

34 mg (43% yield), 95.1% pure by C18-HPLC, MS, C₂₀₀H₂₈₀F₂N₅₂O₆₄S₄[M+2H]²⁺ 2300.9630 found, 2300.9912;

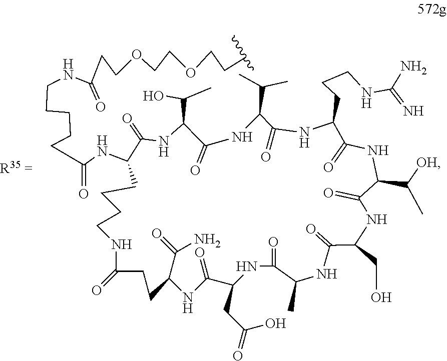 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

40 mg (49% yield), 96.7% pure by C18-HPLC, MS m/z, C₂₁₄H₃₁₀F₂N₅₅O₆₄ [M+2H]²⁺ 2363.1346; found, 2363.1598;

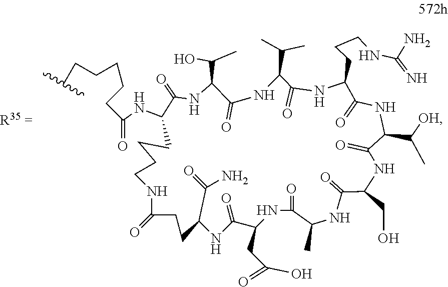 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

38.5 mg (51% yield), 96.9% pure by C18-HPLC, MS m/z, C₂₀₀H₂₈₄F₂N₄O₅₈[M+2H]²⁺ 2204.0451; found, 2204.0677;

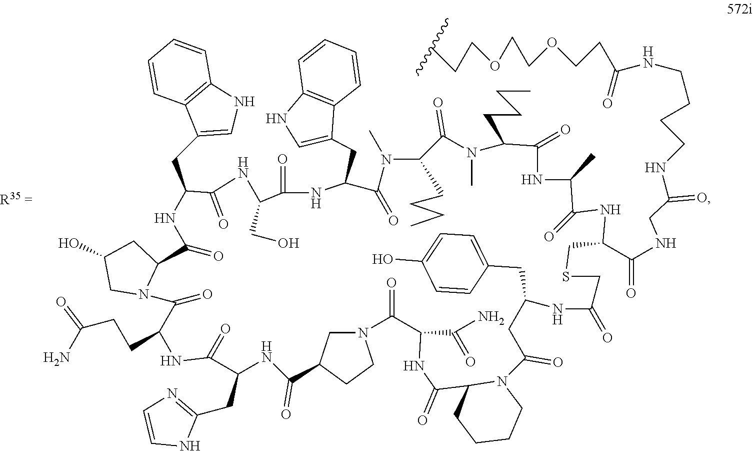 [SMILES: *CCOCCOCCC(=O)NCCCCNC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

50.3 mg (46% yield), 96.0% pure by C18-HPLC, MS m/z, C₃₀₈H₄₀₇F₂N₇₂O₇₄S₂ [M+3H]⁺2133.3236; found, 2133.3585;

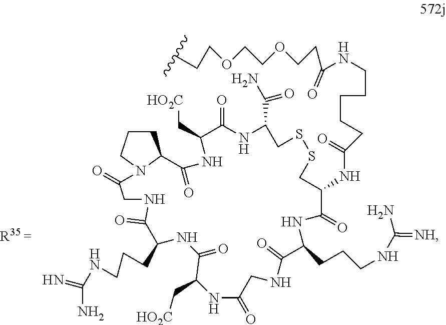 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

41 mg (51% yield), 94.8% pure by C18-HPLC, MS, C₂₀₄H₂₈₆F₂N₆₀O₆₀S₄ [M+2H]²⁺ 2351.0012; found, 2351.0355;

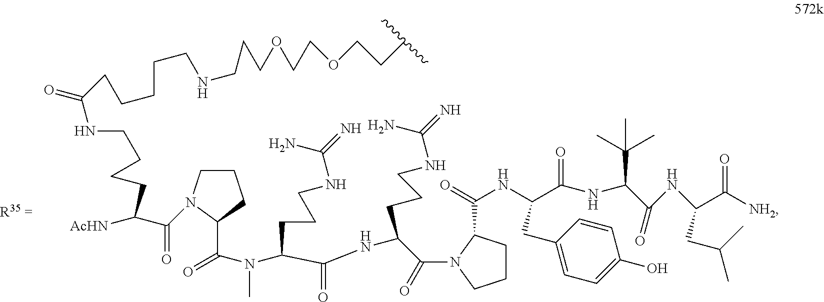 [SMILES: *CCOCCOCCCNCCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(N)=O)C(C)(C)C]

37 mg (44% yield), 96.7% pure by C18-HPLC, MS m/z, C₂₃₈H₃₅₀F₂N₆₀O₅₂ [M+2H]²⁺ 2459.3278; found, 2459.3595;

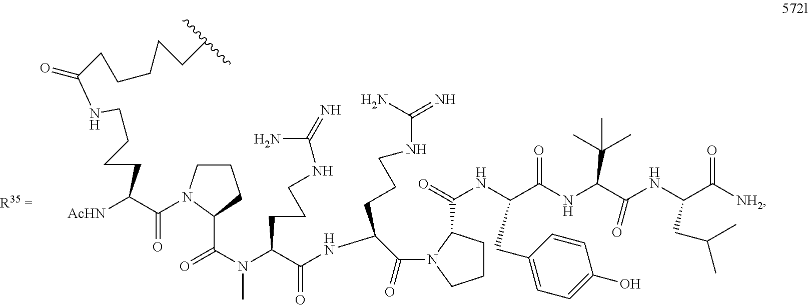

35 mg (44% yield), 96.8% pure by C18-HPLC, MS m/z, C₂₂₄H₃₂₀F₂N₅₈O₄₈ [M+2H]²⁺ 2314.2175; found, 2314.2418;

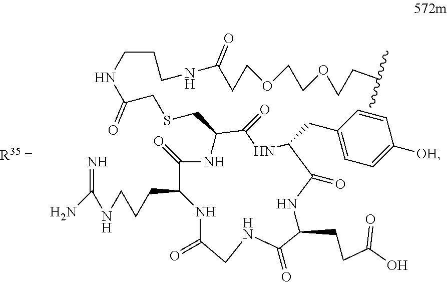 [SMILES: *CCOCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

33 mg (48% yield), 96.5% pure by C18-HPLC, MS m/z, C₁₈₁H₂₄₀F₂N₄₆O₅₀S₂ [M+2H]²⁺ 1985.8530; found, 1985.8872;

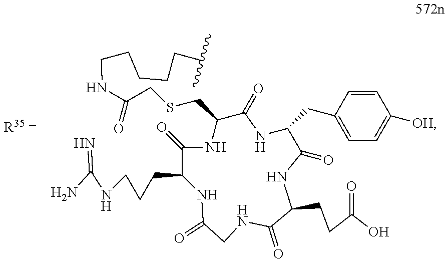 [SMILES: *CCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O]

30 mg (47% yield), 96.2% pure by C18-HPLC, MS m/z, C₁₇₂H₂₂₂F₂N₄₄O₄₄S₂[M+2H]²⁺ 1854.7948; found, 1854.8115;

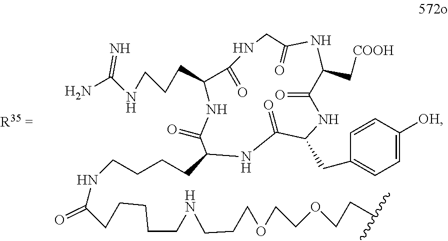 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

34 mg (50% yield), 96.6% pure by C18-HPLC, MS m/z, C₁₈₉H₂₅₈F₂N₄₆O₄₈ [M+2H]²⁺ 1988.9565; found, 1988.9832;

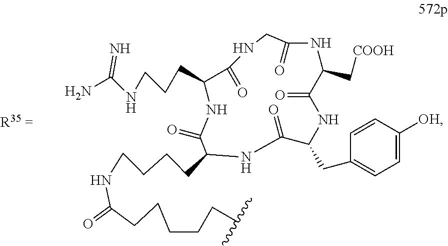 [SMILES: *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

35 mg (55% yield), 96.8% pure by C18-HPLC, MS m/z, C₁₇₄H₂₂₆F₂N₄₄O₄₄ [M+2H]²⁺ 1836.8384; found, 1836.8695.

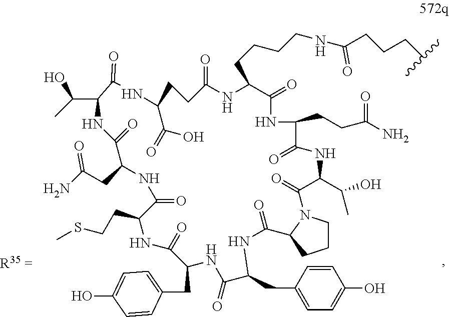 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

44 mg (52% yield), 95.3% pure by C18-HPLC, MS, C₂₂₈H₂₉₈F₂N₅₂O₆₄S₂[M+2H]²⁺ 2445.0536; found, 2445.0887.

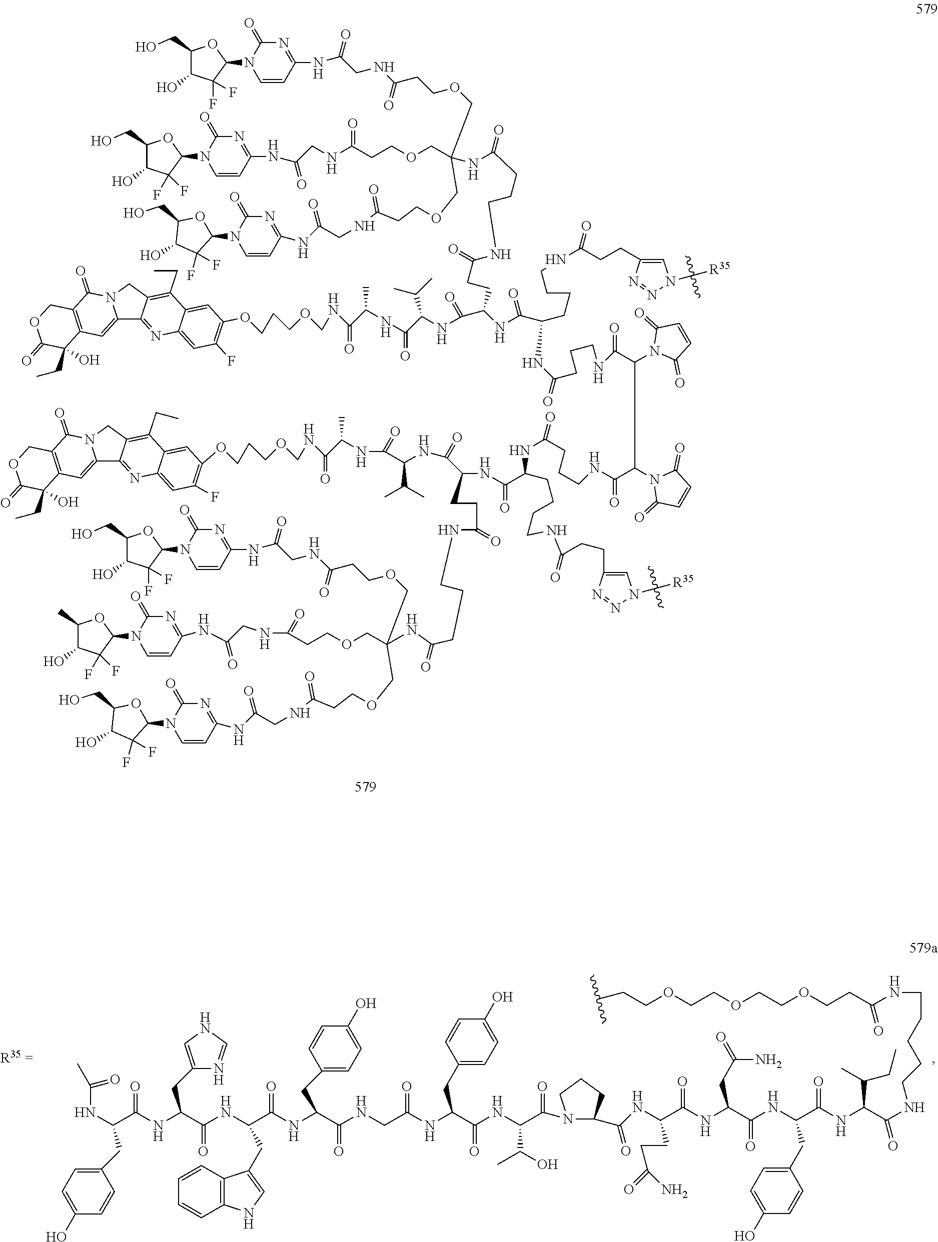

35 mg (45% yield), 95.0% pure by HPLC, MS-ESI (m/z): [M+4H]⁴⁺ calcd for: C₄₁₀H₅₃₂F₁₄N₉₂O₁₂₄, 2248.4482; found: 2248.4753;

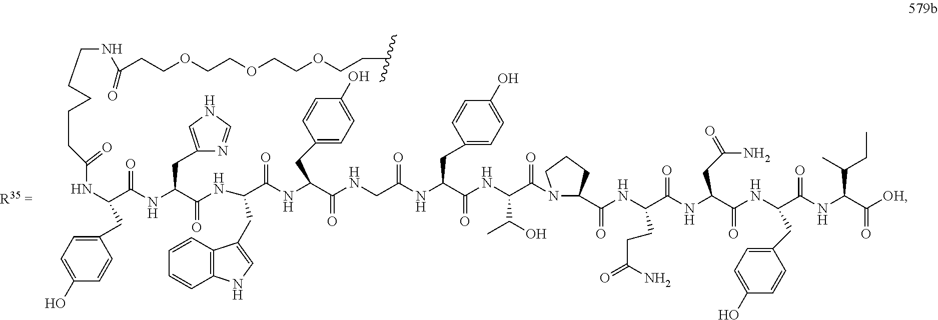 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

36 mg (47% yield), 94.3% pure by HPLC, MS-ES I (m/z): [M+4H]⁴⁺ calcd for: C₄₀₈H₅₂₆F₄N₉O₂₆, 2241.9324; found: 2241.9589;

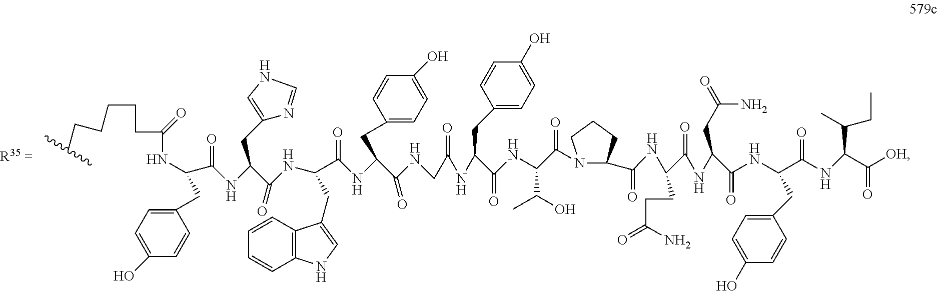 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

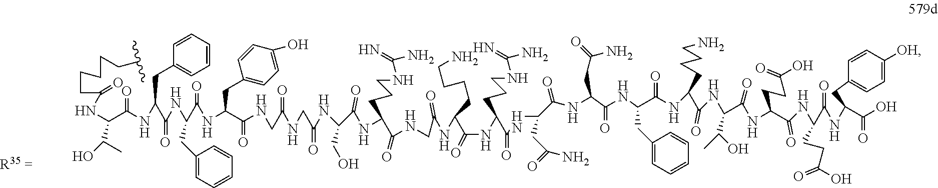 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

40 mg (475% yield), 94.% pure, MS-ESI (m/z): [M+4H]⁴⁺ calcd for: C₄₄₀H₉₆F₁₄NN₂O₁₄a, 2488.5681; found: 2488.5945.

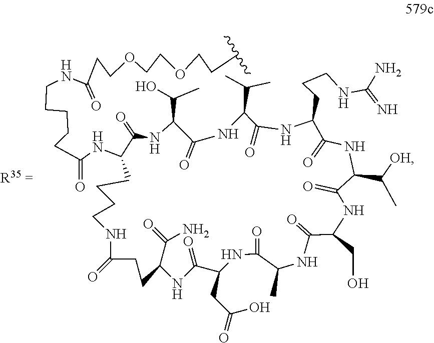

35 mg (53% yield), 95.0% pure by C18-HPLC, MS m/z, C₃₂₆H₄₆₄F₁₄N₅₄O₁₁₄ [M+4H]⁴⁺ 1911.3217; found, 1911.3485;

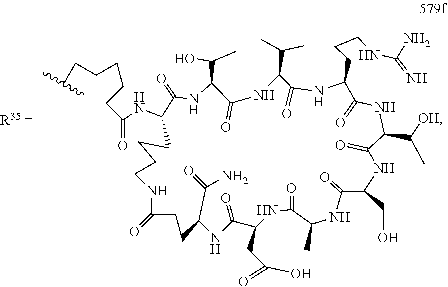 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

35 mg (55% yield), 95.1% pure by C18-HPLC, MS m/z, C₃₁₂H₄₃₈F₁₄N₈₂O₁₀₈ [M+4H]⁴⁺ 1831.7770; found, 1831.8013;

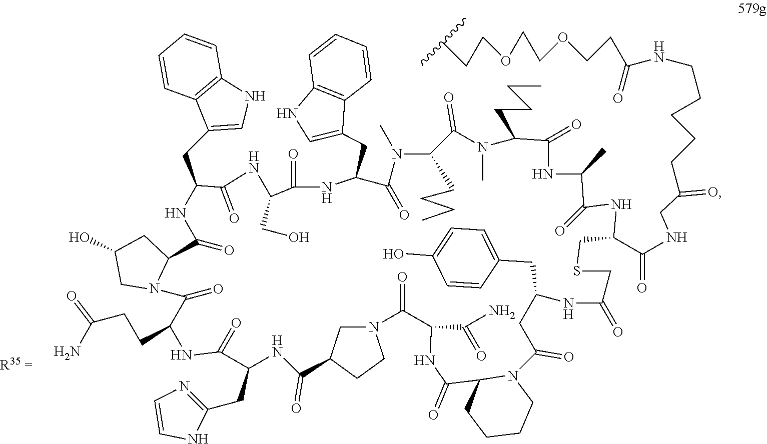 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

40 mg (50% yield), 95.0% pure by C18-HPLC, MS m/z, C₄₂₀H₅₆₀F₁₄N₁₀₀O₁₂₄S₂[M+4H]⁴⁺ 2329.4951; found, 2329.5215;

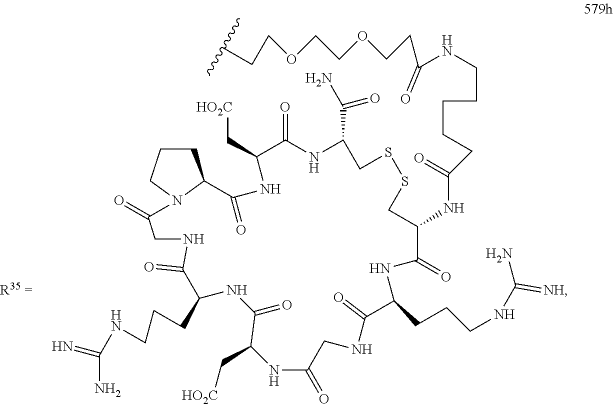 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCNC(=N)N)NC(=O)[C@H](CC(=O)O)NC(=O)CNC(=O)[C@H](CCCNC(=N)N)NC1=O]

34 mg (52% yield), 94.0% pure by C18-HPLC, MS, C₃₁₆H₄₄₀F₁₄N₈₈8 M+4H]⁴⁺ 1905.2550; found, 1905.2797;

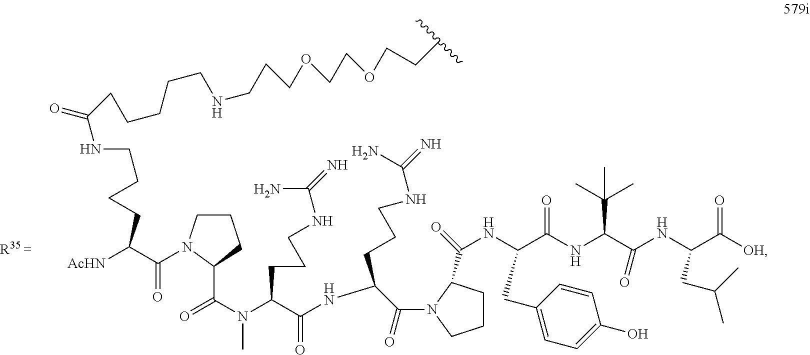

5 mg (52% yield), 95.0% pure by C18-HPLC, MS m/z, C₃₅₀H₅₀₂F₁₄N₈₆O₁₀₄ [M+4H]⁴⁺ 1959.9103; found, 1959.9381;

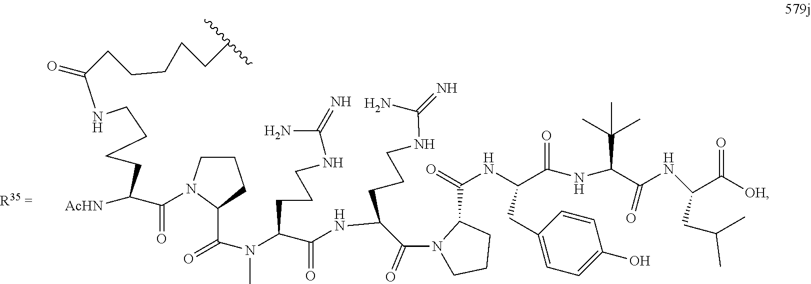

36 mg (55% yield), 95.3% pure by C18-HPLC, MS m/z, C₃₃₆H₄₂₇F₁₄N₈₄O₁₀₀ [M+4H]⁴⁺ 1887.3552; found, 1887.3802;

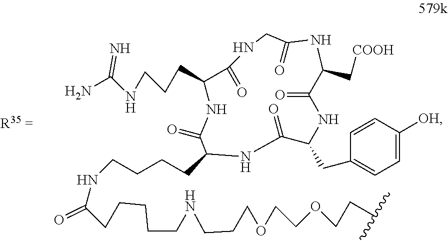 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

32 mg (54% yield), 95.3% pure by C18-HPLC, MS m/z, C₃₀₀H₄₀₉F₁₄N₇₄O₉₈ [M+3H]³+2293.9691; found, 2293.9917;

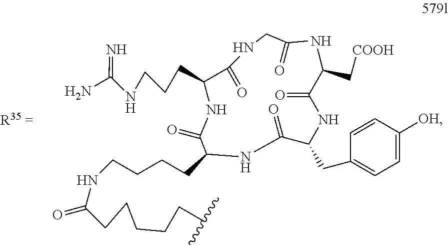 [SMILES: *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

31 mg (57% yield), 95.4% pure by C18-HPLC, MS m/z, C₂₈₆H₃₇₉F₁₄N₇₂O₉₄ [M+3H]³⁺ 2197.2289; found, 2197.2535;

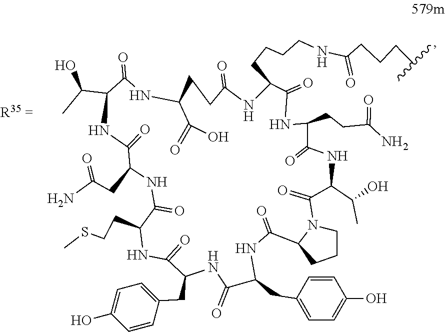 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

39 mg (58% yield), 95.0% pure by C18-HPLC, MS, C₃₄₀H₄₅₂F₁₄N₈₀O₁₁₄S₂[M+4H]⁴⁺ 1952.2812; found, 1952.3084.

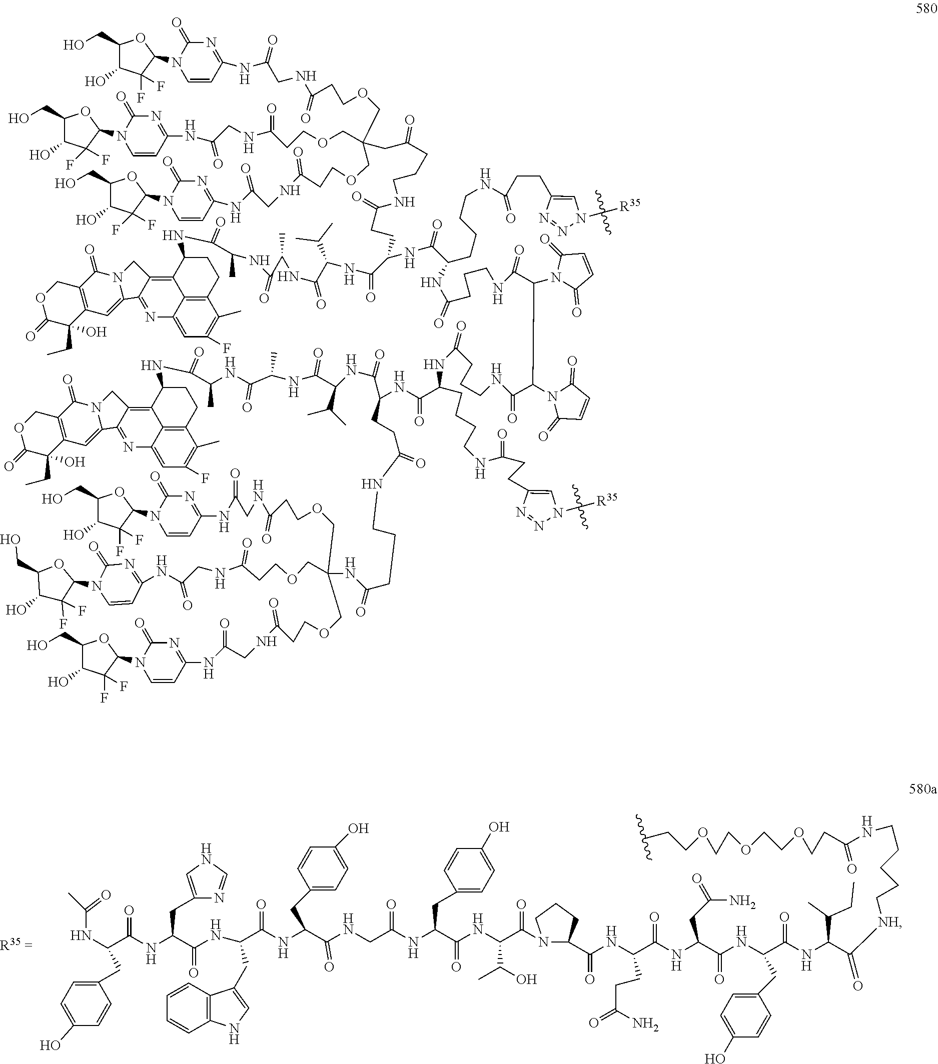

36 mg (46io yield), 95.94 pure by HPLC, MS-ESI (m/z): [M+4H]⁴⁺ calcd for: C₄₁₂H₅₃₀F₁₄N₉₄O₁₂₂, 2252.9484; found: 2252.9735;

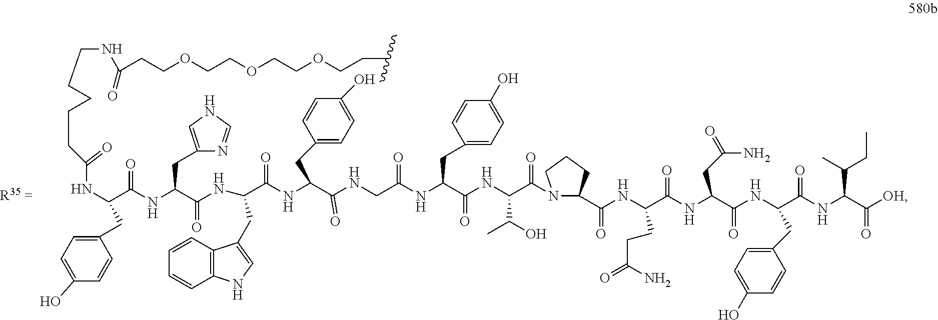 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

36 mg (47% yield), 94.8% pure by HPLC, MS-ESI (m/z): [M+4H]⁴⁺ calcd for: C₄₁₀H₅₂₄F₄N₉₂O₁₂₄, 2246.4325; found: 2246.4596;

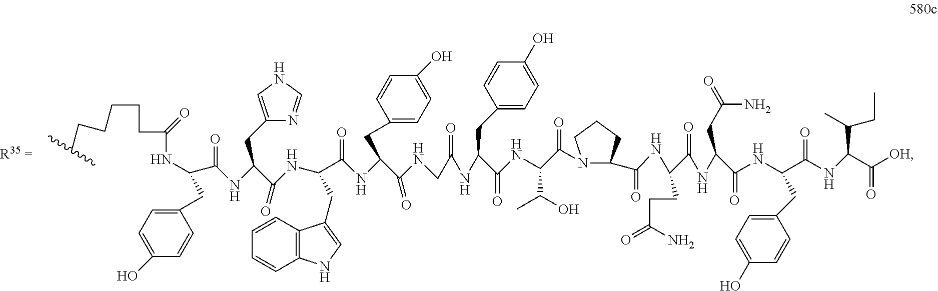 [SMILES: *CCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

36.5 mg, (49% yield), 95.5% pure by HPLC, MS-ESI (m/z): [M+4H]⁴⁺ calcd for: C₃₉₂H₄₉₀F₁₄N₉₀O₁₁₆, 2144.8747 found: 2144.9012;

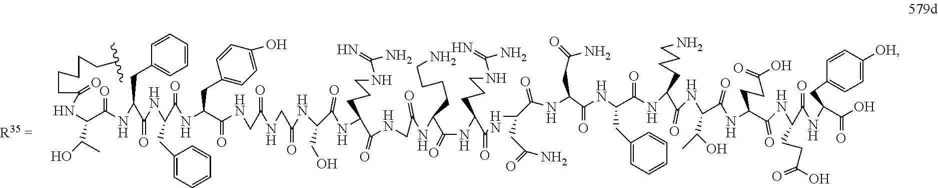 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

41 mg (48% yield), 94.3% pure, MS-ESI (m/z): [M+4H]⁴⁺ calcd for: C₄₄₂H₅₉₄F₁₄N₄O₁₃s, 2493.0686; found: 2493.0945;

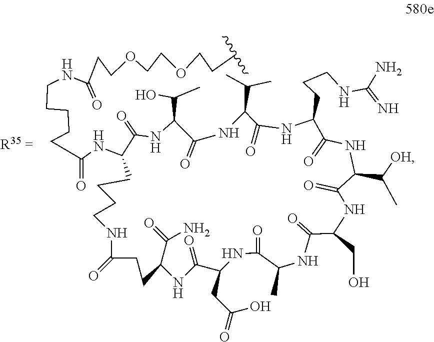 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

35 mg (52% yield), 95.3% pure by C18-HPLC, MS m/z, C₃₂₈H₄₆₂F₁₄N₅₆O₁₁₂ [M+4H]⁴⁺ 1915.8219; found, 1915.8467;

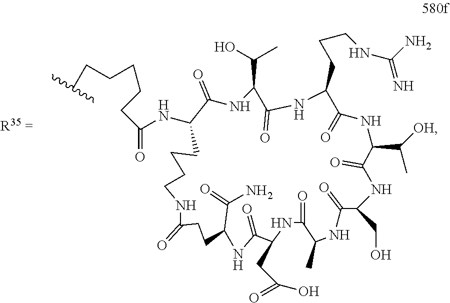 [SMILES: *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)O)NC1=O]

36 mg (57% yield), 95.4% pure by C18-HPLC, MS m/z, C₃₁₄H₄₃₆F₁₄N₅₄O₁₀₆ [M+4H]⁴⁺ 1836.2771; found, 1836.3023;

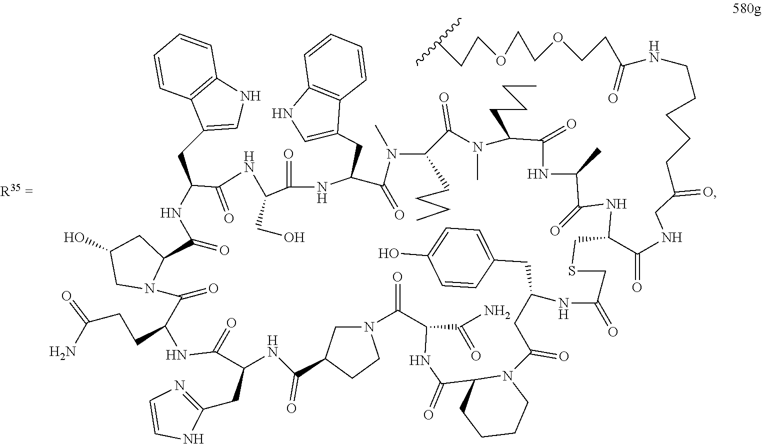 [SMILES: *CCOCCOCCC(=O)NCCCCCC(=O)CNC(=O)[C@@H]1CSCC(=O)N[C@@H](Cc2ccc(O)cc2)CC(=O)N2CCCC[C@@H]2C(=O)N[C@@H](C(N)=O)C(=O)N2CC[C@H](C2)C(=O)N[C@@H](Cc2ncc[nH]2)C(=O)N[C@@H](CCC(N)=O)C(=O)N2C[C@H](O)C[C@H]2C(=O)N[C@@H](Cc2c[nH]c3ccccc23)C(=O)N[C@@H](CO)C(=O)N[C@@H](Cc2c[nH]c3ccccc23)C(=O)N(C)[C@@H](CCCC)C(=O)N(C)[C@@H](CCCC)C(=O)N[C@@H](C)C(=O)N1]

41 mg (51% yield), 95.1% pure by C18-HPLC, MS m/z, C₄₂₂H₅₅₈F₁₄N₁₀₂O₁₂₂S₂[M+4H]⁴⁺ 2333.9953; found, 2334.0216;

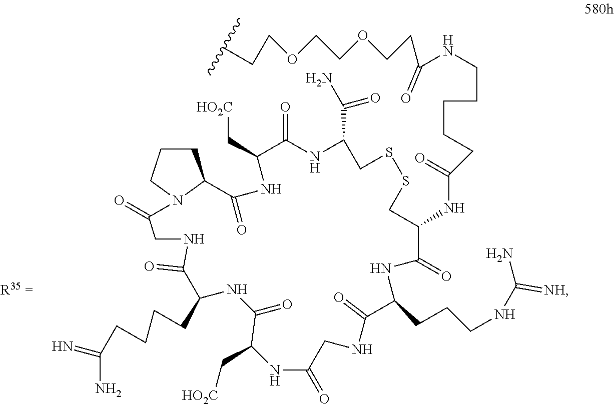 [SMILES: *CCOCCOCCC(=O)NCCCCCC(=O)N[C@H]1CSSC[C@@H](C(N)=O)NC(=O)[C@H](CC(=O)O)NC(=O)[C@@H]2CCCN2C(=O)CNC(=O)[C@H](CCCCC(=N)N)NC(=O)[C@H](CC(=O)O)NC(=O)CNC(=O)[C@H](CCCNC(=N)N)NC1=O]

35 mg (53% yield), 94.1% pure by C18-HPLC, MS, C₃₁₈H₄₃₈F₁₄N₉₀O₁₀₅S₄ [M+4H]⁴⁺ 2 1909.7552; found, 1909.7799;

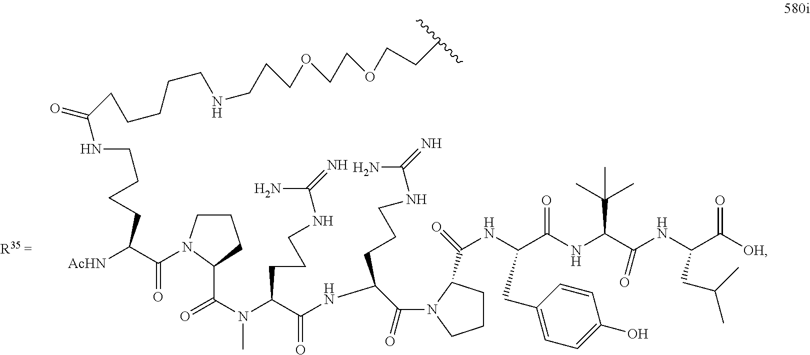

36 mg (530 yield), 95.2% pure by C18-HPLC, MS m/z, C₃₈₂H₅₀₀F₁₄N₈₈O₁₀₂ [M+4H]⁴⁺ 1964.4105; found, 1964.4373;

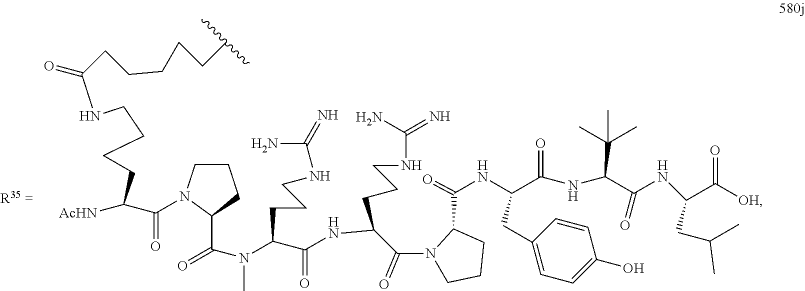 [SMILES: *CCCCCC(=O)NCCCC[C@H](NC(C)=O)C(=O)N1CCC[C@H]1C(=O)N(C)[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C]

37 mg (56% yield), 95.4% pure by C18-HPLC, MS m/z, C₃₃₈H₄₂₈F₁₄N₈₈O₉₈ [M+4H]⁴⁺ 1880.5173; found, 1880.5410;

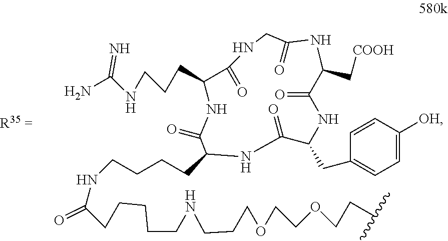 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

33 mg (55% yield), 95.3% pure by C18-HPLC, MS m/z, C₃₀₂H₄₃₇F₁₄N₆O₄[M+3H]³⁺ 2299.9693; found, 2299.9945;

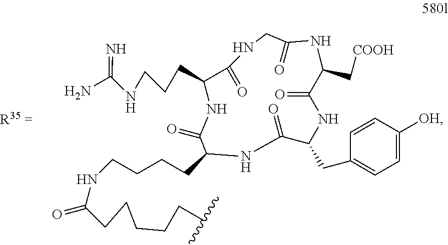 [SMILES: *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O]

31 mg (57% yield), 95.4% pure by C18-HPLC, MS m/z, C₂₈₈H₃₇₇F₁₄N₇₄O₉₂ [M+3H]hu 3+ 2203.2291; found, 2203.2538;

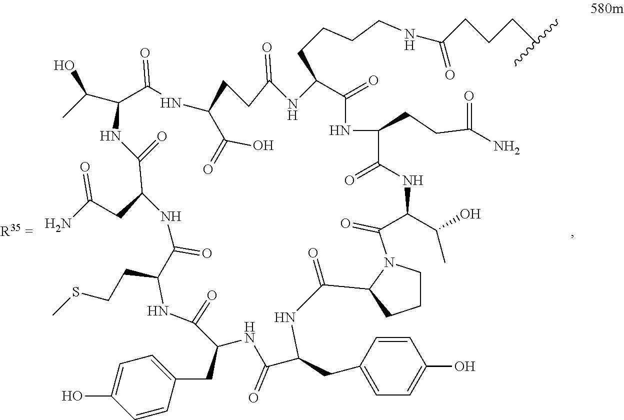 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

40 mg (59% yield), 95.1% pure by C18-HPLC, MS, C₃₄₂H₄₅₀F₁₄N₈₂O₁₁₂S₂[M+4H]⁴⁺ 1956.7814; found, 1956.8065.

### Example 401. Synthesis of (9H-fluoren-9-yl)methyl (S)-(1-((4-(hydroxymethyl)phenyl)-amino)-1-oxopropan-2-yl)carbamate (583)

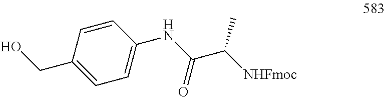

To a solution of (((9H-fluoren-9-yl)methoxy)carbonyl)-L-alanine (581) (7.58 g, 24.35 mmol, 1.0 eq) and (4-aminophenyl)methanol (582) (3 g, 24.35 mmol, 1.0 eq) in THF (50 mL) was added EEDQ (7.23 g, 29.24 mmol, 1.2 eq) at r.t. The mixture was stirred at RT overnight, then concentrated in vacuo, and slurried with addition of EA/PE (1:5) to afford 583 (8.57 g, 85% yield) as a white solid.

### Example 402. Synthesis of (S)-2-amino-N-(4-(hydroxymethyl)phenyl)propanamide (584)

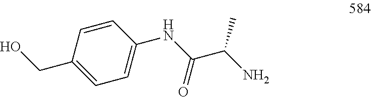

To a solution of 583 (2.1 g, 5.04 mmol) in DMF (10 mL) was added piperidine (1 mL). The mixture was stirred at RT for 20 min and then concentrated in vacuo to afford 584 (O_{.98} g, crude) as a white solid, which was used for the next step without further purification.

### Example 403. Synthesis of (9H-fluoren-9-yl)methyl ((S)-1-(((S)-1-((4-(hydroxymethyl)-phenyl)amino)-1-oxopropan-2-yl)amino)-3-methyl-1-oxobutan-2-yl)carbamate (586)

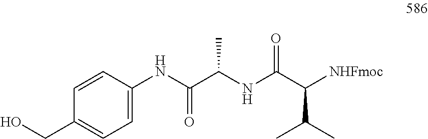

To a solution of 584 (O_{.98} g, 5.046 mmol, 1.0 eq) and (((9H-fluoren-9-yl)methoxy)-carbonyl)-L-valine (585) (1.71 g, 5.046 mmol, 1.0 eq) in THF (10 mL) was added EEDQ (1.87 g, 7.568 mmol, 1.5 eq) at r.t. The mixture was stirred at RT overnight and then concentrated in vacuo. The residue was purified by column chromatography on silica gel (eluted with DCM/MeOH=10/1) to afford 586 (2.1 g, 81% yield in two steps) as a white solid. ESI: m/z: calcd for C₃₀H₃₃N₃O₅ [M+H]⁺: found 516.7.

### Example 404. Synthesis of (9H-fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (588)

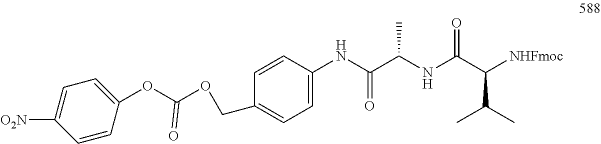

To a solution of 586 (1.33 g, 2.579 mmol, 1.0 eq) and 587 (O_{.68} g, 3.353 mmol, 1.3 eq) in THF (10 mL) was added pyridine (O_{.41} g, 5.159 mmol, 2.0 eq) at r.t. The mixture was stirred at RT for 3 h and then extracted with DCM (20 mL×3) and H₂O (20 mL). The organic layers were washed with 10% citric acid solution (20 mL×2), dried over anhydrous Na₂SO₄ and concentrated in vacuo. The residue was purified by column chromatography on silica gel (eluted with DCM/EA=2/1) to afford 588 (716 mg, 41% yield) as a white solid. ESI: m/z: calcd for C₃₇H₃₆N₄O₉ [M+H]⁺: found 681.8.

### Example 405. Synthesis of (9H-fluoren-9-yl)methyl ((S)-1-(((S)-1-((4-((((1-((2R,4R,5R)-3,3-difluoro-4-hydroxy-5-(hydroxymethyl)tetrahydrofuran-2-yl)-2-oxo-1, 2-dihydropyrimidin-4-yl)carbamoyl)oxy)methyl)phenyl)amino)-1-oxopropan-2-yl)amino)-3-methyl-1-oxobutan-2-yl)carbamate (590)

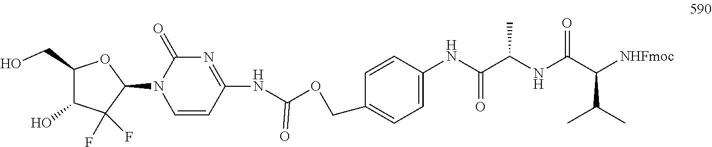

To a solution of 588 (716 mg, 1.05 mmol) and 589 (Gemcitabine) HCl salt (314 mg, 1.05 mmol) in THF (35 mL) was added DIPEA (321 mg, 3.15 mmol, 3.0 eq) at r.t. The mixture was stirred at 65° C. overnight, concentrated in vacuo and purified by column chromatography on silica gel (eluted with DCM/MeOH=15/1110/) to afford 590 (414 mg, 49% yield) as a white solid. ESI: m/z: calcd for C₄₀H₄₂F₂N₆O₁₀ [M+H]⁺: found 805.8.

### Example 406. Synthesis of 4-((S)-2-((S)-2-amino-3-methylbutanamido)propanamido)-benzyl (1-((2R,4R,5R)-3,3-difluoro-4-hydroxy-5-(hydroxymethyl)tetrahydrofuran-2-yl)-2-oxo-1,2-dihydropyrimidin-4-yl)carbamate (591)

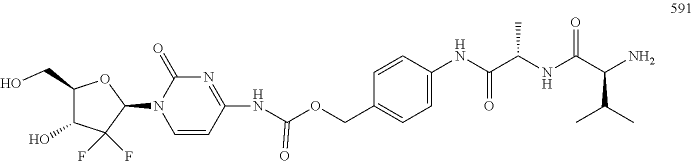 [SMILES: CC(C)[C@H](N)C(=O)N[C@@H](C)C(=O)Nc1ccc(COC(=O)Nc2ccn([C@@H]3O[C@H](CO)[C@@H](O)C3(F)F)c(=O)n2)cc1]

To a solution of 590 (414 mg, 0.51 mmol) in DMF (4 mL) was added piperidine (O_{.4} mL). The mixture was stirred at RT for 20 min and then concentrated in vacuo to afford 591 (297 mg, crude) as a white solid, which was used for the next step without further purification. ESI: m/z: calcd for C₂₅H₃₂F₂N₆O₈[M+H]⁺: found 583.6.

### Example 407. Synthesis of 11,11-bis((2-carboxyethoxy)methyl)-2,2-dimethyl-4,9-dioxo-3,13-dioxa-5,10-diazahexadecan-16-oic acid (594)

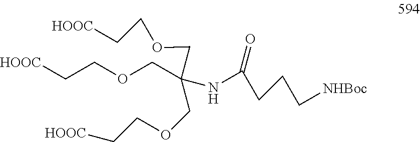 [SMILES: CC(C)(C)OC(=O)NCCCC(=O)NC(COCCC(=O)O)(COCCC(=O)O)COCCC(=O)O]

To a stirring solution of 12 (283 mg, 0.84 mmol) in DMF (5 ml) at room temperature were added 13 (252 mg, 0.84 mmol) and DIEA (254 mg, 2.00 mmol). The mixture was stirred for 2 h at room temperature and then purified by pre-C18 HPLC (d30×L250 cm, eluted with 5% CH₃CN in water to 60% CH₃CN in water at 15 ml/min in 45 min) to afford 594 (263 mg, 60% yield) as a white solid. C₂₂H₃₈N₂O₁₂ [M+H]⁺: found 523.6.

### Example 408. Synthesis of tert-butyl ((2S,5S)-1-((4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)-oxy)methyl)phenyl)amino)-12,12-bis((3-(((S)-1-(((S)-1-((4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)-oxy)methyl)phenyl)amino)-1-oxopropan-2-yl)amino)-3-methyl-1-oxobutan-2-yl)amino)-3-oxopropoxy)methyl)-5-isopropyl-2-methyl-1,4,7,14-tetraoxo-10-oxa-3,6,13-triazaheptadecan-17-yl)carbamate (595)

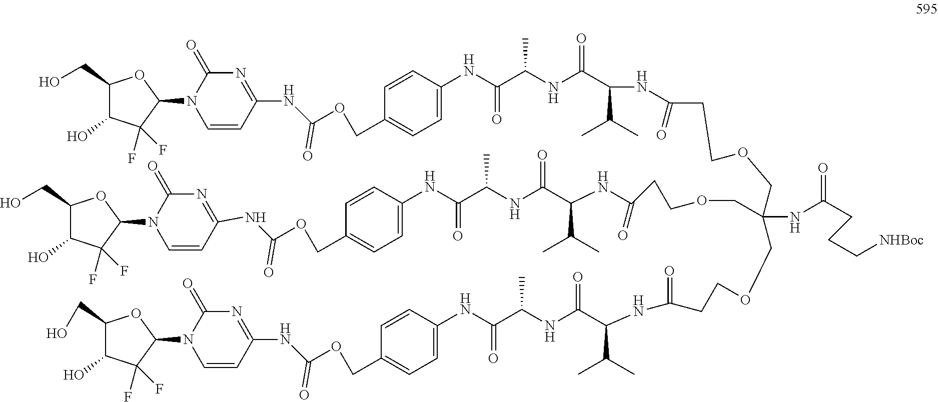 [SMILES: CC(C)[C@H](NC(=O)CCOCC(COCCC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)(COCCC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)NC(=O)CCCNC(=O)OC(C)(C)C)C(=O)N[C@@H](C)C(=O)Nc1ccc(COC(=O)Nc2ccn([C@@H]3O[C@H](CO)[C@@H](O)C3(F)F)c(=O)n2)cc1]

To a solution of 594 (67 mg, 0.128 mmol, 1.0 eq) and 591 (297 mg, 0.51 mmol, 4.0 eq) in DMF (4 ml) at room temperature were added HATU (160 mg, 0.42 mmol, 3.3 eq) and DIPEA (99 mg, 0.768 mmol, 6.0 eq). The reaction mixture was stirred for 0.5 h at room temperature, concentrated and purified by pre-C18 HPLC (d30×L250 cm, eluted with 5% CH₃CN in water to 60% CH₃CN in water at 15 m₁/min in 45 min) to afford 595 (153 mg, 54% yield) as a white solid. ESI: m/z: calcd for C₉₇H₁₂₈F₆N₂₀O₃₃ [M+H]⁺: found 2217.2.

### Example 409. Synthesis of 3,3′-((2-(1-amino-3,6,9,12,15,18,21,24-octaoxaheptacosan-27-amido)-2-((3-((2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-2-oxoethyl)amino)-3-oxopropoxy)methyl)propane-1,3-diyl)bis(oxy))bis(N-(2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-2-oxoethyl)propanamide) (596)

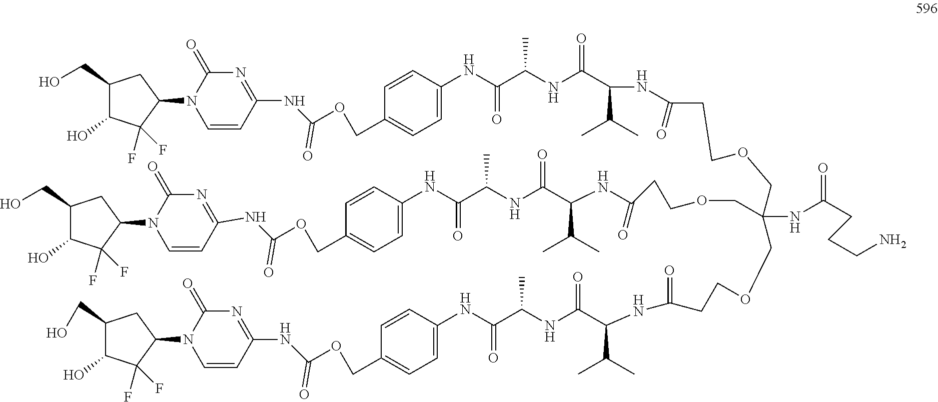 [SMILES: CC(C)[C@H](NC(=O)CCOCC(COCCC(=O)N[C@H](C(=O)N[C@@H](C)C(=O)Nc1ccc(COC(=O)Nc2ccn([C@@H]3C[C@H](CO)[C@@H](O)C3(F)F)c(=O)n2)cc1)C(C)C)(COCCC(=O)N[C@H](C(=O)N[C@@H](C)C(=O)Nc1ccc(COC(=O)Nc2ccn([C@@H]3C[C@H](CO)[C@@H](O)C3(F)F)c(=O)n2)cc1)C(C)C)NC(=O)CCCN)C(=O)N[C@@H](C)C(=O)Nc1ccc(COC(=O)Nc2ccn([C@@H]3C[C@H](CO)[C@@H](O)C3(F)F)c(=O)n2)cc1]

To a stirring solution of 595 (153 mg, 0.069 mmol) in DCM (5 mL) was added TFA (2 mL) at room temperature. The resulting mixture was stirred at room temperature for 1 h, and concentrated in vacuo. After addition of EA (20 ml), a suspension was filtered, and afford 596 TFA salt (148 mg, crude) as a white solid. ESI: m/z: calcd for C₉₂H₁₂₀F₆N₂₀O₃₁ [M+H]⁺: found 2117.1.

### Example 410. Synthesis of (37S,45S)-45-(3-(tert-butoxy)-3-oxopropyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazahexatetracontan-46-oic acid (599)

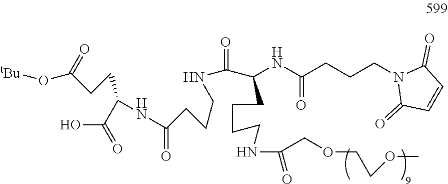

To a solution of 597 (20 g, 23.122 mmol) and NHS (3.99 g, 34.683 mmol, 1.5 eq) in DCM (100 mL) was added EDCI (8.86 g, 46.244 mmol, 2.0 eq). The mixture was stirred at RT for 1 h, washed with H₂O (100 mL×2). The organic layers were dried over anhydrous Na₂SO₄ and concentrated in vacuo to afford the succinimide, which was then diluted with DMF (100 mL). 588 (4.65 g, 23.122 mmol, 1.0 eq) and DIPEA (2.98 g, 23.122 mmol, 1.0 eq) was added to the solution. The mixture was stirred at RT overnight and then extracted with EA (200 mL×2) and H₂O (200 mL). The aqueous layer was acidified with 1 N HCl solution to pH=3, then extracted with DCM (200 mL×3). The organic layers were washed with brine (200 mL×2), dried over anhydrous Na₂SO₄ and concentrated in vacuo to afford 599 (18.6 g, 77% yield) as a yellow oil. ESI: m/z: calcd for C₄₈H₈₃N₅O₂₀ [M+H]⁺: found 1051.2.

### Example 411. Synthesis of tert-butyl ((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-3-methyl-I-oxobutan-2-yl)carbamate (602)

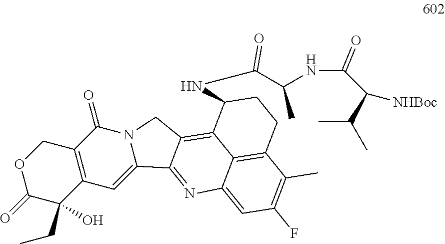 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)OC(C)(C)C)C(C)C)CC3]

To a solution of 601 ((S)-2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propanamide) TFA salt (511 mg, 0.823 mmol, 1.0 eq) and 21 ((tert-butoxycarbonyl)-L-valine) (179 mg, 0.823 mmol, 1.0 eq) in DMF (5 mL) were added HATU (376 mg, 0.988 mmol, 1.2 eq) and DIPEA (319 mg, 2.47 mmol, 3.0 eq). The reaction mixture was stirred at RT for 1.5 h, then extracted with DCM (30 mL×2) and H₂O (30 mL). The combined organic layers were washed with brine, dried over anhydrous Na₂SO₄, concentrated in vacuo and purified by column chromatography on silica gel (eluted with DCM/MeOH=20/1) to afford 602 (545 mg, 94% yield) as a yellow solid. ESI: m/z: calcd for C₃₇H₄₄FN₅O₈[M+H]⁺: found 706.8.

### Example 412. Synthesis of(S)-2-amino-N-((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)-3-methylbutanamide (603)

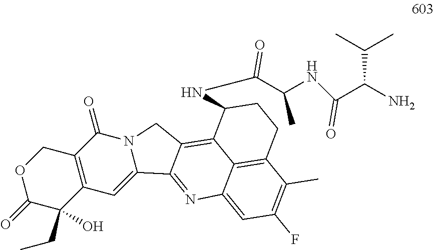 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](N)C(C)C)CC3]

To a solution of 602 (545 mg, 0.772 mmol, 1.0 eq) in DCM (10 mL) was added TFA (4 mL).The mixture was stirred at 1 h, then concentrated in vacuo. The residue was slurred with MTBE to afford 603 TFA salt (555 mg, crude) as a yellow solid. ESI: m/z: calcd for C₃₂H₃₆FN₅O₆[M+H]⁺: found 606.8.

### Example 413. Synthesis of tert-butyl (37S,45S)-37-(4-(2,5-dioxo-2,5-dihydro-1H-pyrrol-1-yl)butanamido)-45-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-3-methyl-1-oxobutan-2-yl)carbamoyl)-31,38,43-trioxo-2,5,8,11,14,17,20,23,26,29-decaoxa-32,39,44-triazaoctatetracontan-48-oate (604)

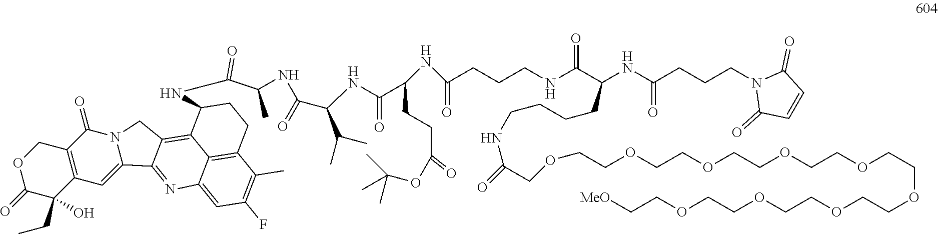 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)[C@H](CCC(=O)OC(C)(C)C)NC(=O)CCCNC(=O)[C@H](CCCCNC(=O)COCCOCCOCCOCCOCCOCCOCCOCCOCCOC)NC(=O)CCCN1C(=O)C=CC1=O)C(C)C)CC3]

To a solution of 599 (538 mg, 0.772 mmol, 1.0 eq) in DMF (5 mL) was added HATU (352 mg, 0.926 mmol, 1.2 eq). The mixture was stirred at RT for 10 min, 603 TFA salt (555 mg, 0.772 mmol, 1.0 eq, crude) and DIPEA (299 mg, 2.316 mmol, 3.0 eq) were added to the reaction. The mixture was stirred at RT for 0.5 h and purified by prep-C18 HPLC (d30×L250 cm, eluted with 35% CH₃CN in water to 80% CH₃CN in water at 15 ml/min in 50 min) to afford 604 (1.01 g, 80% yield) as a white solid. ESI: m/z: calcd for C₈₀H₁₁₇FN₁₀O₂₅ [M+H]⁺: found 1638.9.

### Example 414. Synthesis of(37S,45S)-37-(4-(2,5-dioxo-2,5-dihydro-IH-pyrrol-1-yl)butanamido)-45-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1-oxopropan-2-yl)amino)-3-methyl-1-oxobutan-2-yl)carbamoyl)-31,38,43-trioxo-2,5,8,11,14,17,20,23,26,29-decaoxa-32,39,44-triazaoctatetracontan-48-oic acid (25)

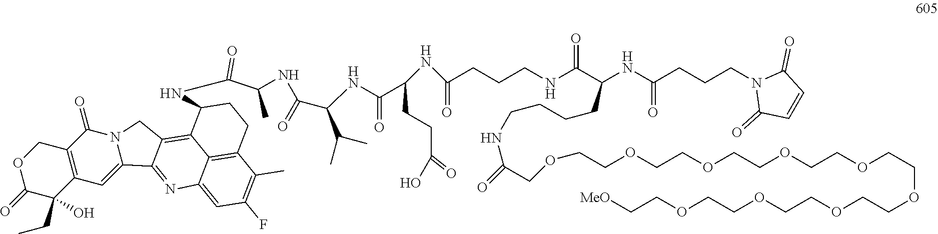 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)[C@H](CCC(=O)O)NC(=O)CCCNC(=O)[C@H](CCCCNC(=O)COCCOCCOCCOCCOCCOCCOCCOCCOCCOC)NC(=O)CCCN1C(=O)C=CC1=O)C(C)C)CC3]

To a solution of 604 (500 mg, 0.305 mmol, 1.0 eq) in DCM (10 mL) was added TFA (10 mL). The mixture was stirred at 1 h, concentrated in vacuo and slurred with MTBE to afford 25 (482 mg, crude) as a yellow solid. ESI: m/z: calcd for C₇₆H₁₀₉FN₁₀O₂₅ [M+H]⁺: found 1582.8.

### Example 415. Synthesis of compound (606)

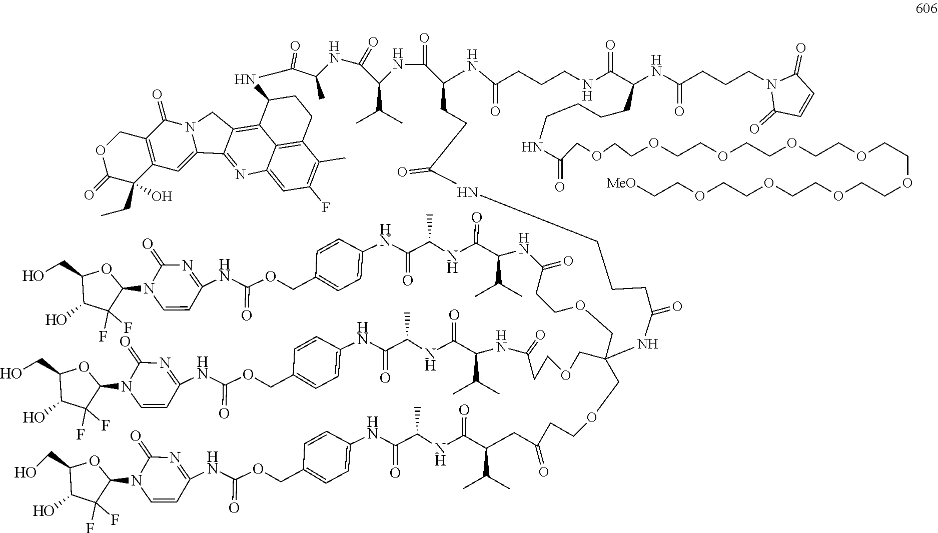

To a solution of 605 (105 mg, 0.066 mmol, 1.0 eq) in DMF (5 mL) was added HATU (60 mg, 0.158 mmol, 2.4 eq). The mixture was stirred at RT for 10 min. 596 TFA salt (148 mg, 0.066 mmol, 1.0 eq, crude) and DIPEA (26 mg, 0.198 mmol, 3.0 eq) were added to the reaction. The mixture was stirred at RT for 0.5 h and purified by prep-C18 HPLC (d30×L250 cm, eluted with 10% CH₃CN in water to 70% CH₃CN in water at 15 ml/min in 50 min) to afford 606 (124 mg, 51% yield) as a white solid. ESI MS m/z: [2M+H]⁺ calcd for C₁₆₈H₂₂₇F₇N₃₀O₅₅: found 1841.0.

### Example 416. Synthesis of 4-((S)-2-((S)-2-((((9H-fluoren-9-yl)methoxy)carbonyl)amino)-3-methylbutanamido)propanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (609)

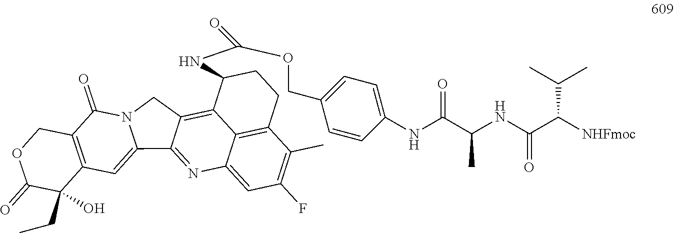

To a solution of 588 (500 mg, 0.73 mmol, 1.0 eq) and exatecan CH₃SO₂H salt (388 mg, 0.73 mmol, 1.0 eq) in DMF (35 mL) was added DIPEA (283 mg, 2.19 mmol, 3.0 eq) at r.t. The mixture was stirred at RT for 2 h, concentrated in vacuo and purified by column chromatography on silica gel (eluted with DCM/MeOH=20/1~10/1) to afford 609 (435 mg, 61% yield) as a yellow solid. ESI MS m/z: [M+H]⁺ calcd for C₅₅H₅₃FN₆O₁₀: found 978.0.

### Example 417. Synthesis of 4-((S)-2-((S)-2-amino-3-methylbutanamido)propanamido)-benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (610)

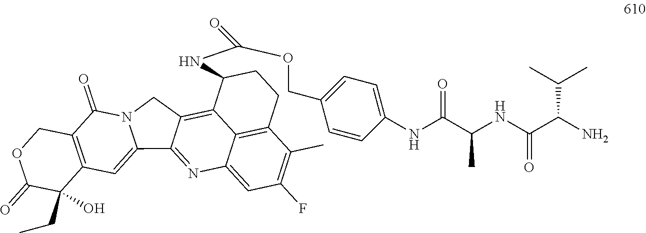 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](N)C(C)C)cc1)CC3]

To a solution of 609 (435 mg, 0.45 mmol) in DMF (4 mL) was added piperidine (O_{.4} mL). The mixture was stirred at RT for 20 min and purified by prep-C18 HPLC ((d30×L250 cm, eluted with 20% CH₃CN in water to 80% CH₃CN in water containing 0.1% TFA at 15 ml/min in 50 min) to afford 610 TFA salt (344 mg, 88%) as a yellow solid. ESI MS m/z: [M+H]⁺ calcd for C₄₀H₄₃FN₆O₈: found 755.8.

### Example 418. Synthesis of tert-butyl (37S,45S)-37-(4-(2,5-dioxo-2,5-dihydro-1H-pyrrol-1-yl)butanamido)-45-(((S)-1-(((S)-I-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-31,38,43-trioxo-2,5,8,11,14,17,20,23,26,29-decaoxa-32,39,44-triazaoctatetracontan-48-oate (611)

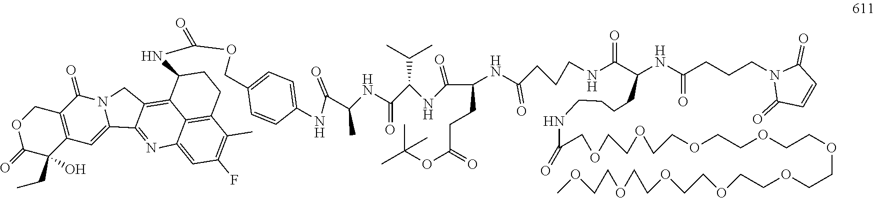 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)OC(C)(C)C)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a solution of 599 (276 mg, 0.396 mmol, 1.0 eq) in DMF (5 mL) was added HATU (180 mg, 0.475 mmol, 1.2 eq), 610 TFA salt (344 mg, 0.396 mmol, 1.0 eq, crude) and DIPEA (153 mg, 1.188 mmol, 3.0 eq). The mixture was stirred at RT for 3.5 h and then purified by prep-HPLC (d30×L250 cm, eluted with 30% CH₃CN in water to 90% CH₃CN in water at 15 ml/min in 50 min) to afford 611 (587 mg, 83% yield) as a white solid. ESI MS m/z: [M+H]⁺ calcd for C₈₈H₁₂₄FN₁₁O₂₇: found 1788.1.

### Example 419. Synthesis of (37S,45S)-37-(4-(2,5-dioxo-2,5-dihydro-1H-pyrrol-1-yl)butanamido)-45-(((S)-1-(((S)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-31,38,43-trioxo-2,5,8,11,14,17,20,23,26,29-decaoxa-32,39,44-triazaoctatetracontan-48-oic acid (612)

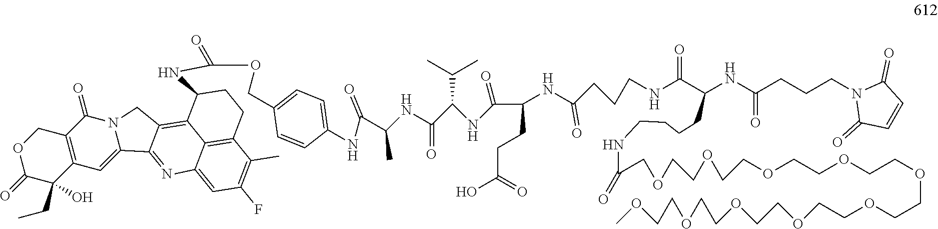 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)O)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a solution of 611 (587 mg, 0.328 mmol, 1.0 eq) in DCM (10 mL) was added TFA (5 mL). The mixture was stirred at 1 h. The reaction mixture was concentrated in vacuo. The residue was slurred with MTBE to afford 612 (567 mg, crude) as a white solid. ESI MS m/z: [M+H]⁺ calcd for C₈₄H₁₁₆FN₁O₂₇: found 1731.9.

### Example 420. Synthesis of compound (613)

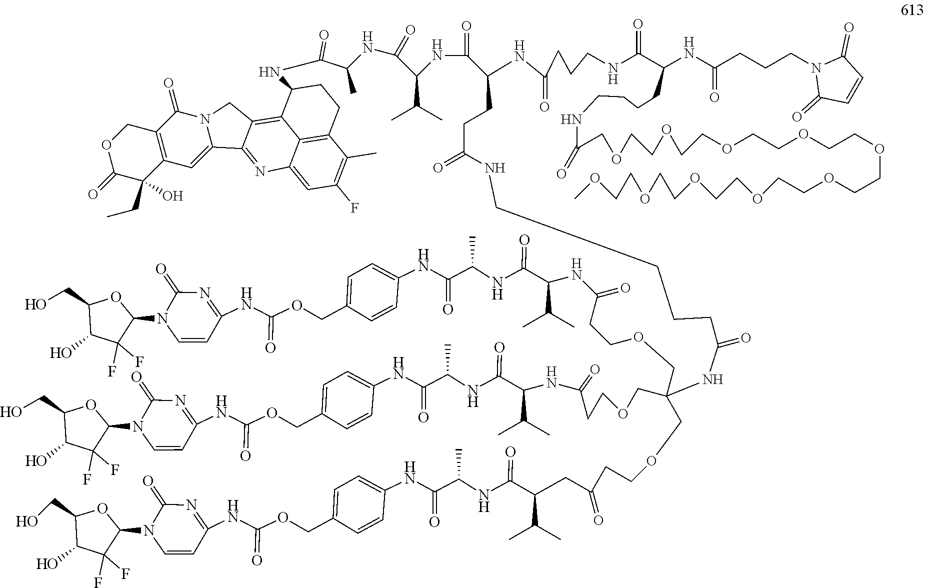

To a solution of 612 (567 mg, 0.33 mmol, 1.0 eq) in DMF (5 mL) was added HATU (150 mg, 0.40 mmol, 1.2 eq), 596 TFA salt (740 mg, 0.33 mmol, 1.0 eq, crude) and DIPEA (128 mg, 0.99 mmol, 3.0 eq). The mixture was stirred at RT for 2.5 h and purified by prep-C18 HPLC (d30×L250 cm, eluted with 10% CH₃CN in water to 800% CH₃CN in water at 15 mi/min in 50 min) to afford 613 (682 mg, 54% yield) as a white solid. ESI MS m/z: [2M+H]⁺ calcd for C₁₇₆H₂₃₄F₇N₃₁O₅O₇: found 1915.5.

### Example 421. Synthesis of 3,3′-((4-((tert-butoxycarbonyl)amino)butanoyl)azanediyl)-dipropionic acid (616)

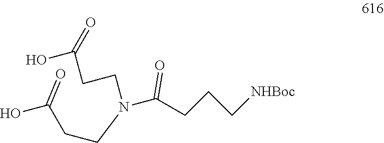 [SMILES: CC(C)(C)OC(=O)NCCCC(=O)N(CCC(=O)O)CCC(=O)O]

To a stirring solution of 615 (3,3′-azanediyldipropionic acid) (135 mg, 0.84 mmol) in DMF (5 m₁) at room temperature were added 593 (252 mg, 0.84 mmol) and DIEA (254 mg, 2.00 mmol). The mixture was stirred for 2 h at room temperature and purified by pre-C18 HPLC (d30×L250 cm, eluted with 5% CH₃CN in water to 70% CH₃CN in water at 15 ml/min in 50 min) to afford 616 (276 mg, 95% yield) as a white solid. ESI MS m/z: [M+H]⁺ calcd for C₁₅H₂₆N₂O₇: found 347.4.

### Example 422. Synthesis of 4-((15S,18S)-10-(3-(((S)-1-(((S)-1-((4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-1-oxopropan-2-yl)amino)-3-methyl-1-oxobutan-2-yl)amino)-3-oxopropyl)-15-isopropyl-2,2,18-trimethyl-4,9,13,16-tetraoxo-3-oxa-5,10,14,17-tetraazanonadecan-19-amido)benzyl (1-((2R,4R,5R)-3,3-difluoro-4-hydroxy-5-(hydroxymethyl)-tetrahydrofuran-2-yl)-2-oxo-1,2-dihydropyrimidin-4-yl)carbamate (617)

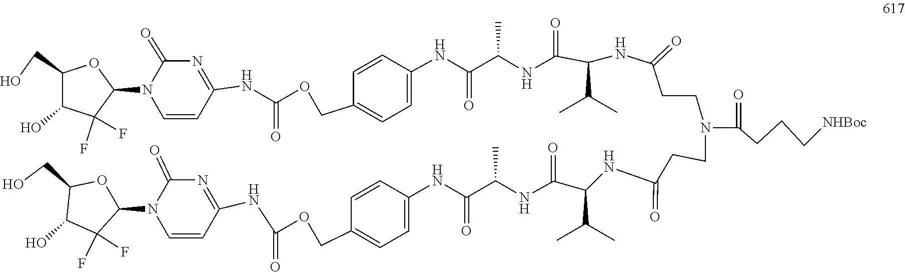 [SMILES: CC(C)[C@H](NC(=O)CCN(CCC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)C(=O)CCCNC(=O)OC(C)(C)C)C(=O)N[C@@H](C)C(=O)Nc1ccc(COC(=O)Nc2ccn([C@@H]3O[C@H](CO)[C@@H](O)C3(F)F)c(=O)n2)cc1]

To a solution of 616 (276 mg, 0.797 mmol, 1.0 eq) and 591 (1.393 g, 2.391 mmol, 3.0 eq) in DMF (4 ml) at room temperature were added HATU (666 mg, 1.753 mmol, 2.2 eq) and DIPEA (411 mg, 3.188 mmol, 4.0 eq). The reaction mixture was stirred for 2.5 h at room temperature and purified by pre-C18 HPLC (d30×L250 cm, eluted with 5% CH₃CN in water to 70% CH₃CN in water at 15 ml/min in 50 min) to afford 617 (988 mg, 84% yield) as a white solid. ESI MS m/z: [M+H]⁺ calcd for C₆₅H₈₆F₄N₁₄O₂₁: found 1476.5.

### Example 423. Synthesis of((((2S,5S,15S,18S)-10-(4-aminobutanoyl)-5,15-diisopropyl-2,18-dimethyl-4,7,13,16-tetraoxo-3,6,10,14,17-pentaazanonadecanedioyl)bis(azanediyl))bis(4,1-phenylene))bis(methylene)bis((1-((2R,4R,5R)-3,3-difluoro-4-hydroxy-5-(hydroxymethyl)-tetrahydrofuran-2-yl)-2-oxo-1,2-dihydropyrimidin-4-yl)carbamate), TFA salt (618)

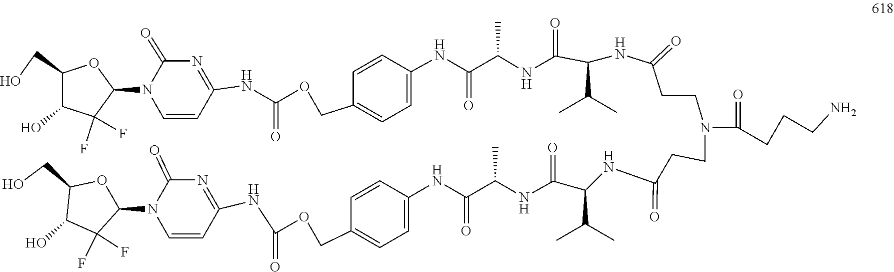 [SMILES: CC(C)[C@H](NC(=O)CCN(CCC(=O)N[C@H](C(=O)N[C@@H](C)C(=O)Nc1ccc(COC(=O)Nc2ccn([C@@H]3O[C@H](CO)[C@@H](O)C3(F)F)c(=O)n2)cc1)C(C)C)C(=O)CCCN)C(=O)N[C@@H](C)C(=O)Nc1ccc(COC(=O)Nc2ccn([C@@H]3O[C@H](CO)[C@@H](O)C3(F)F)c(=O)n2)cc1]

To a solution of 617 (988 mg, 0.700 mmol) in DCM (10 mL) was added TFA (3 mL). The mixture was stirred at RT for 0.5 h and concentrated in vacuo to afford 38 (1.04 g, crude). ESI MS m/z: [M+H]⁺calcd for C₆₀H₇₈F₄N₁₄O₁₉: found 1376.4.

### Example 424. Synthesis of compound (619)

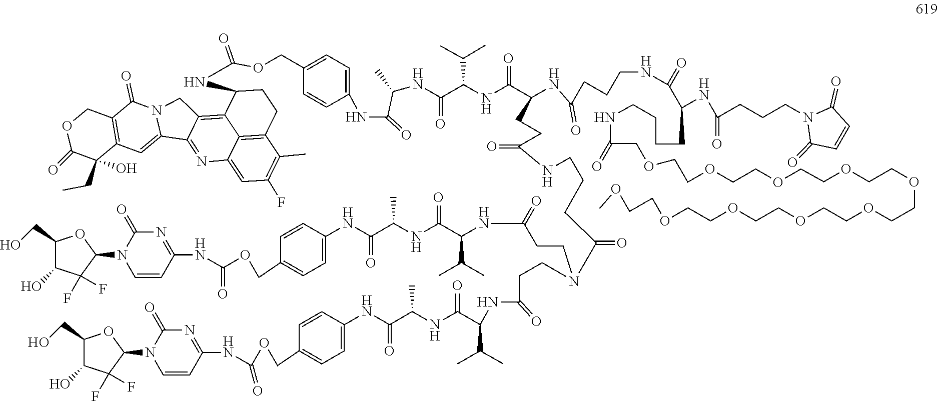 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)NCCCC(=O)N(CCC(=O)N[C@H](C(=O)N[C@@H](C)C(=O)Nc2ccc(COC(=O)Nc4ccn([C@@H]5O[C@H](CO)[C@@H](O)C5(F)F)c(=O)n4)cc2)C(C)C)CCC(=O)N[C@H](C(=O)N[C@@H](C)C(=O)Nc2ccc(COC(=O)Nc4ccn([C@@H]5O[C@H](CO)[C@@H](O)C5(F)F)c(=O)n4)cc2)C(C)C)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a solution of 612 (567 mg, 0.33 mmol, 1.0 eq) in DMF (5 mL) was added HATU (150 mg, 0.40 mmol, 1.2 eq), 618 TFA salt (491 mg, 0.33 mmol, 1.0 eq, crude) and DIPEA (128 mg, 0.99 mmol, 3.0 eq). The mixture was stirred at RT for 3 h and then purified by pre-C18 HPLC (d30×L250 cm, eluted with 5% CH₃CN in water to 70% CH₃CN in water at 15 ml/min in 50 min) to afford 619 (580 mg, 57% yield) as a white solid. ESI MS m/z: [2M+H]⁺calcd for C₁₄₄H₁₉₂F₅N₂₅O₄₅: found 1545.1.

### Example 425. Synthesis of 4-((37S,45S,48S,51S)-45-(3-(((S)-1-(((S)-1-((4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-1-oxopropan-2-yl)amino)-3-methyl-1-oxobutan-2-yl)amino)-3-oxopropyl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (621)

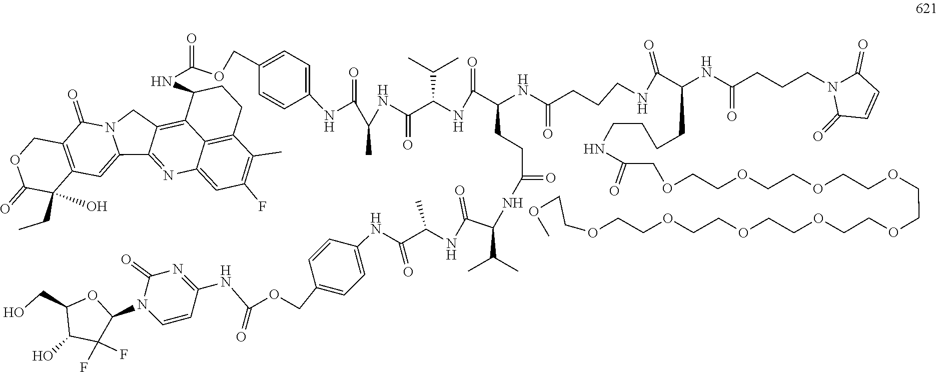 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)N[C@H](C(=O)N[C@@H](C)C(=O)Nc2ccc(COC(=O)Nc4ccn([C@@H]5O[C@H](CO)[C@@H](O)C5(F)F)c(=O)n4)cc2)C(C)C)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a solution of 612 (56.7 mg, 0.033 mmol, 1.0 eq) in DMF (2 mL) was added HATU (15 mg, 0.040 mmol, 1.2 eq), 591 TFA salt (19 mg, 0.033 mmol, 1.0 eq, crude) and DIPEA (12.8 mg, 0.099 mmol, 3.0 eq). The mixture was stirred at RT for 2.5 h and then purified by pre-C18 HPLC (d30×L250 cm, eluted with 5% CH₃CN in water to 80% CH₃CN in water at 15 ml/min in 50 min) to afford 621 (46 mg, 61% yield) as a white solid. ESI MS m/z: [2M+H]⁺ calcd for C₁₀₀H₁₄₆F₃N₁₇O₃₄: found 1148.8.

### Example 426. Synthesis of ((2R,3R,5R)-5-(4-amino-2-oxopyrimidin-1(2H)-yl)-3-((tert-butoxycarbonyl)oxy)-4,4-difluorotetrahydrofuran-2-yl)methyl tert-butyl carbonate (623)

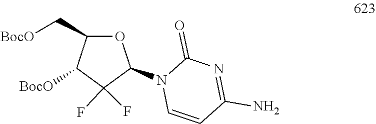

To a solution of 589 (10 g, 37.994 mmol, 1.0 eq) and KOH (21.32 g, 379.94 mmol, 10.0 eq) in H₂O (380 mL) was added (Boc)₂₀ (82.92 g, 379.94 mmol, 10.0 eq). The mixture was stirred at room temperature for 30 min. KOH (21.32 g, 379.94 mmol, 10.0 eq) in H₂O (380 mL) was added to the reaction. The mixture was stirred at RT for 20 min and then extracted with EA (500 mL×3). The organic layers were washed with brine, dried over anhydrous Na₂SO₄, concentrated in vacuo, and purified by column chromatography on silica (DCM/MeOH=20/1) to afford 623 (10.97 g, 62%) as a white solid. ESI MS m/z: [M+H]⁺calcd for C₁₉H₂₇F₂N₃₀₈: found 464.5.

### Example 427. Synthesis of tert-butyl (2-((2-((2-((1-((2R,4R,5R)-4-((tert-butoxycarbonyl)-oxy)-5-(((tert-butoxycarbonyl)oxy)methyl)-3,3-difluorotetrahydrofuran-2-yl)-2-oxo-1,2-dihydropyrimidin-4-yl)amino)-2-oxoethyl)amino)-2-oxoethyl)amino)-2-oxoethyl)carbamate (625)

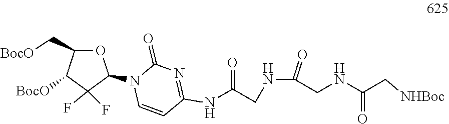

To a solution of 623 (500 mg, 1.08 mmol, 1.0 eq) and 624 (312 mg, 1.08 mmol, 1.0 eq) in CH₃CN (10 ml) were added TCFH (485 mg, 1.728 mmol, 1.6 eq) and NMI (266 mg, 3.24 mmol, 3.0 eq). The mixture was stirred at RT for 2 h, concentrated in vacuo. The residue was purified by column chromatography on silica (DCM/MeOH=20/1) to afford 625 (706 mg, 89%) as a white solid. ESI MS m/z: [M+H]⁺calcd for C₃₀H₄₄F₂N₆O₁₃: found 735.8.

### Example 428. Synthesis of 2-amino-N-(2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)-amino)-2-oxoethyl)acetamide (626)

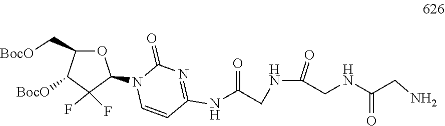

To a stirring solution of 625 (706 mg, 0.96 mmol, 1.0 eq) in CH₃CN (5 mL) was added TMSI (384 mg, 1.92 mmol, 2.0 eq). The mixture was stirred at RT for 15 min, then quenched with H₂O (34 mg, 1.92 mmol, 2.0 eq), and filtered. The filter cake was washed with CH₃CN and dried in vacuo to afford 626 HI salt (539.8 mg, crude) as a yellow solid. ESI MS m/z: [M+H]⁺calcd for C₁₅H₂₀F₂N₆07: found 435.4.

### Example 429. Synthesis of tert-butyl (1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-15,15-bis(14-((1-((2R,4R,5R)-3,3-difluoro-4-hydroxy-5-(hydroxymethyl)tetrahydrofuran-2-yl)-2-oxo-1,2-dihydropyrimidin-4-yl)amino)-5,8,11,14-tetraoxo-2-oxa-6,9,12-triazatetradecyl)-1,4,7,10,17-pentaoxo-13-oxa-3,6,9,16-tetraazaicosan-20-yl)carbamate (627)

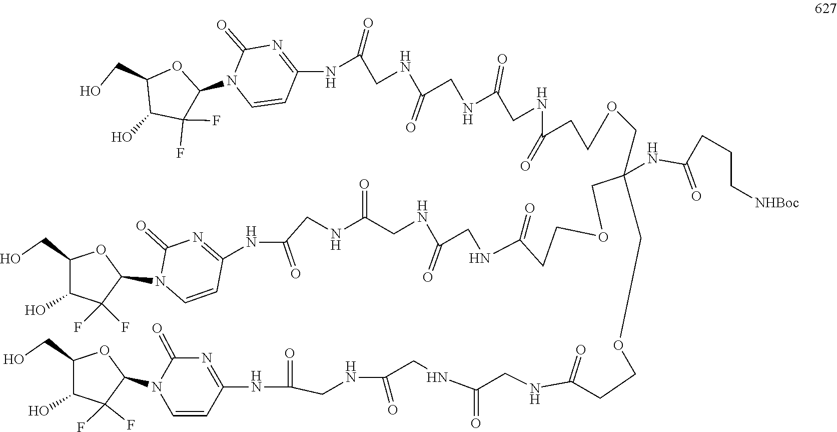 [SMILES: CC(C)(C)OC(=O)NCCCC(=O)NC(COCCC(=O)NCC(=O)NCC(=O)NCC(=O)Nc1ccn([C@@H]2O[C@H](CO)[C@@H](O)C2(F)F)c(=O)n1)(COCCC(=O)NCC(=O)NCC(=O)NCC(=O)Nc1ccn([C@@H]2O[C@H](CO)[C@@H](O)C2(F)F)c(=O)n1)COCCC(=O)NCC(=O)NCC(=O)NCC(=O)Nc1ccn([C@@H]2O[C@H](CO)[C@@H](O)C2(F)F)c(=O)n1]

To a solution of 626 HI salt (216 mg, 0.384 mmol, 4.0 eq) and 594 (50 mg, 0.096 mmol, 1.0 eq) in DMF (3 mL) were added HATU (109 mg, 0.288 mmol, 3.3 eq) and DIPEA (99 mg, 0.768 mmol, 8.0 eq). The mixture was stirred at RT for 2.5 h and purified by pre-C18 HPLC (d30×L250 cm, eluted with 5% CH₃CN in water to 70% CH₃CN in water at 15 ml/min in 50 min) to afford 627 (98 mg, 58% yield) as a white solid. ESI MS m/z: [M+H]⁺calcd for C₆₇H₉₂F₆N₂₀O₃₀: found 1772.6.

### Example 430. Synthesis of 3,3′-((2-(4-aminobutanamido)-2-(14-((1-((2R,4R,5R)-3,3-difluoro-4-hydroxy-5-(hydroxymethyl)tetrahydrofuran-2-yl)-2-oxo-1,2-dihydropyrimidin-4-yl)amino)-5,8,11,14-tetraoxo-2-oxa-6,9,12-triazatetradecyl)propane-1,3-diyl)bis(oxy))bis(N-(2-((2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-2-oxoethyl)amino)-2-oxoethyl) propanamide) (628)

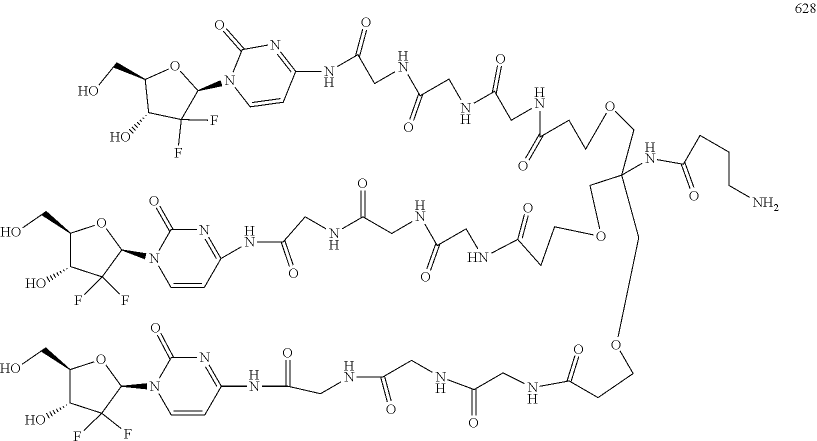 [SMILES: NCCCC(=O)NC(COCCC(=O)NCC(=O)NCC(=O)NCC(=O)Nc1ccn([C@@H]2O[C@H](CO)[C@@H](O)C2(F)F)c(=O)n1)(COCCC(=O)NCC(=O)NCC(=O)NCC(=O)Nc1ccn([C@@H]2O[C@H](CO)[C@@H](O)C2(F)F)c(=O)n1)COCCC(=O)NCC(=O)NCC(=O)NCC(=O)Nc1ccn([C@@H]2O[C@H](CO)[C@@H](O)C2(F)F)c(=O)n1]

To a stirring solution of 627 (98 mg, 0.055 mmol, 1.0 eq) in CH₃CN (2 mL) was added TMSI (22 mg, 0.11 mmol, 2.0 eq). The mixture was stirred at RT for 15 min, then quenched with H₂O (1.98 mg, 0.11 mmol, 2.0 eq), and filtered. The filter cake was washed with CH₃CN and dried in vacuo to afford 628 HI salt (98.9 mg, crude) as a yellow solid. ESI MS m/z: [M+H]⁺ calcd for C₆₂H₈₄F₆N₂₀O₂₈: found 1672.5.

### Example 431. Synthesis of compound (629)

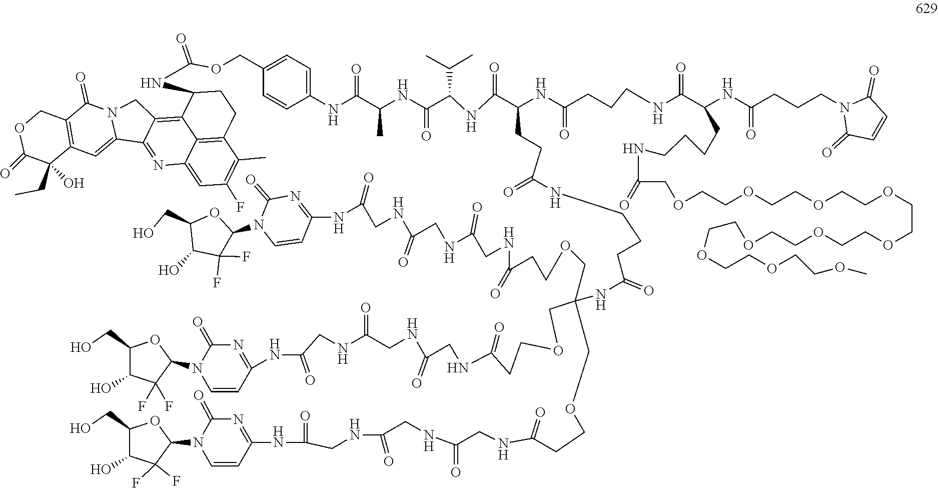 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)NCCCC(=O)NC(COCCC(=O)NCC(=O)NCC(=O)NCC(=O)Nc2ccn([C@@H]4O[C@H](CO)[C@@H](O)C4(F)F)c(=O)n2)(COCCC(=O)NCC(=O)NCC(=O)NCC(=O)Nc2ccn([C@@H]4O[C@H](CO)[C@@H](O)C4(F)F)c(=O)n2)COCCC(=O)NCC(=O)NCC(=O)NCC(=O)Nc2ccn([C@@H]4O[C@H](CO)[C@@H](O)C4(F)F)c(=O)n2)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a solution of 612 (95 mg, 0.055 mmol, 1.0 eq) in DMF (2 mL) was added HATU (25 mg, 0.066 mmol, 1.2 eq), 628 HI salt (98.9 mg, 0.055 mmol, 1.0 eq, crude) and DIPEA (21 mg, 0.165 mmol, 3.0 eq). The mixture was stirred at RT for 2.5 h and purified by pre-C18 HPLC (d30×L250 cm, eluted with 5% CH₃CN in water to 70% CH₃CN in water at 15 ml/min in 50 min) to afford 629 (104 mg, 56% yield in two steps) as a white solid. ESI MS m/z: [2M+H]⁺calcd for C₁₄₆H₁₉₈F₇N₃₁O₅₄: found 1693.2.

### Example 432. Synthesis of tert-butyl (1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-13-(3-((2-((2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-2-oxoethyl)amino)-2-oxoethyl)amino)-3-oxopropyl)-1,4,7,10,14-pentaoxo-3,6,9,13-tetraazaheptadecan-17-yl)carbamate (631)

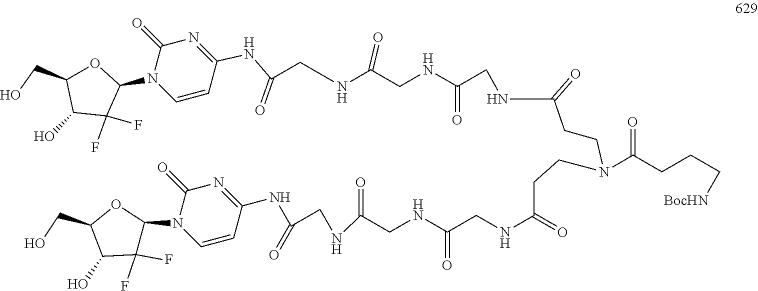 [SMILES: CC(C)(C)OC(=O)NCCCC(=O)N(CCC(=O)NCC(=O)NCC(=O)NCC(=O)Nc1ccn([C@@H]2O[C@H](CO)[C@@H](O)C2(F)F)c(=O)n1)CCC(=O)NCC(=O)NCC(=O)NCC(=O)Nc1ccn([C@@H]2O[C@H](CO)[C@@H](O)C2(F)F)c(=O)n1]

To a stirring solution of 616 (120.0 mg, 0.346 mmol) in DMF (5 mL) at room temperature were added 626 (414.0 mg, 0.954 mmol), HATU (395.19 mg, 1.039 mmol) and DIEA (179.11 mg, 1.386 mmol). The resulting mixture was stirred at room temperature for 15 min before it was concentrated in vacuo. C18 HPLC reverse-phase purification (d30×L250 cm, eluted with 10% CH₃CN in water to 70% CH₃CN in water at 15 ml/min in 50 min) and lyophilization afforded 631 (288.0 mg, yield 70%). ESI: m/z: calcd for C₄₅H₆₂F₄N₁₄O₁₉ [M+H]⁺: found 1180.2.

### Example 433. Synthesis of 4-amino-N,N-bis(3-((2-((2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-2-oxoethyl)amino)-2-oxoethyl)amino)-3-oxopropyl)butanamide (632)

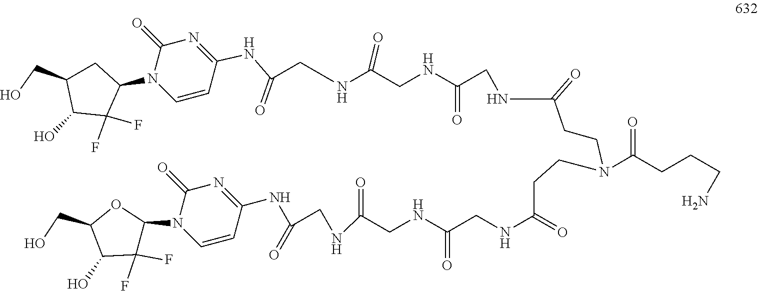 [SMILES: NCCCC(=O)N(CCC(=O)NCC(=O)NCC(=O)NCC(=O)Nc1ccn([C@@H]2C[C@H](CO)[C@@H](O)C2(F)F)c(=O)n1)CCC(=O)NCC(=O)NCC(=O)NCC(=O)Nc1ccn([C@@H]2O[C@H](CO)[C@@H](O)C2(F)F)c(=O)n1]

To a solution of 631 (100.0 mg, 0.094 mmol) in DCM (5 mL) was added TFA (1 mL). The mixture was stirred at room temperature for 1 h and then concentrated in vacuo. The product was triturated from MTBE to afforded 632 (90.6 mg, yield 100). ESI: m/z: calcd for C₄₀H₅₄F₄N₁₄O₁₇ [M+H]⁺: found 1079.9.

### Example 434. Synthesis of 4-((37S,45S,48S,51S)-45-(1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-13-(3-((2-((2-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-2-oxoethyl)amino)-2-oxoethyl)amino)-3-oxopropyl)-1,4,7,10,14,19-hexaoxo-3,6,9,13,18-pentaazahenicosan-21-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (633)

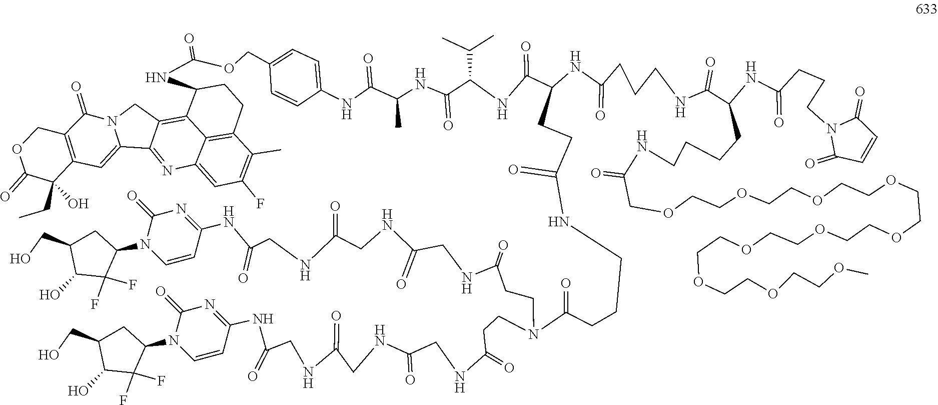 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)NCCCC(=O)N(CCC(=O)NCC(=O)NCC(=O)NCC(=O)Nc2ccn([C@@H]4C[C@H](CO)[C@@H](O)C4(F)F)c(=O)n2)CCC(=O)NCC(=O)NCC(=O)NCC(=O)Nc2ccn([C@@H]4C[C@H](CO)[C@@H](O)C4(F)F)c(=O)n2)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a stirring solution of 632 (54 mg, 0.05 mmol) in DMF (4 ml) at room temperature were added 612 (87 mg, 0.05 mmol), HATU (23 mg, 0.06 mmol) and DIEA (10 mg, 0.08 mmol). The reaction mixture was stirred for 2.5 h at room temperature, and purified by pre-C18 HPLC (d30×L250 cm, eluted with 5% CH₃CN in water to 70% CH₃CN in water at 15 ml/min in 50 min), and lyophilized to afford 633 (48 mg, 34% yield) as a white solid. ESI MS m/z: [2M+H]⁺ calcd for C₁₂₄H₁₆₈F₅N₂₅O₄₃: found 1397.0.

### Example 435. Synthesis of tert-butyl ((S)-1-(((S)-1-((1-((2R,4R,5R)-4-((tert-butoxycarbonyl)oxy)-5-(((tert-butoxycarbonyl)oxy)methyl)-3,3-difluorotetrahydrofuran-2-yl)-2-oxo-1,2-dihydropyrimidin-4-yl)amino)-1-oxopropan-2-yl)amino)-3-methyl-1-oxobutan-2-yl)carbamate (636)

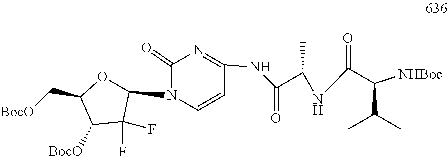

To a stirring solution of 623 (1.0 g, 2.158 mmol) in MeCN (20 mL) at 0° C. was added 635 (O_{.75} g, 2.589 mmol), 1-Methylimidazole (O_{.53} g, 6.473 mmol) and TCFH (O_{.91} g, 3.237 mmol) in sequence. The resulting mixture was stirred 0° C. for 12 h before it was quenched with NaCl. The layers were separated, and the aqueous layer was extracted with CH₂C₁₂. The combined organic layers were dried (Na₂SO₄) and concentrated in vacuo. Flash column chromatography (silica gel, MeOH/DCM=5%-8%) afforded 56 (1.7 g, 100%). HRMS (ESI): calcd for C₃₂H₄₉F₂N₅O₁₂ [M+H]⁺ 733.76, found 733.76.

### Example 436. Synthesis of(S)-2-amino-N-((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)-3-methylbutanamide (637)

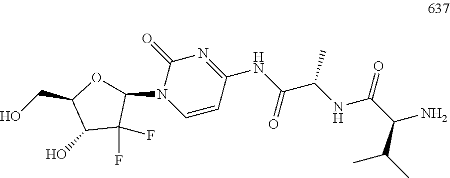 [SMILES: CC(C)[C@H](N)C(=O)N[C@@H](C)C(=O)Nc1ccn([C@@H]2O[C@H](CO)[C@@H](O)C2(F)F)c(=O)n1]

A stirring solution of 636 (O_{.7} g, 0.954 mmol) in DCM (10 mL) and TFA (3 mL) at room temperature. The resulting mixture was stirred at room temperature for 0.5 h before it was concentrated in vacuo. The product was triturated from DCM:EA=1:1 to afforded 637 (425.2 mg, 100%). HRMS (ESI): calcd for C₁₇H₂₆F₂N₅O₆[M+H]⁺ 433.41, found 433.41.

### Example 437. Synthesis of tert-butyl (4-(bis(3-(((S)-1-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-3-methyl-1-oxobutan-2-yl)amino)-3-oxopropyl)amino)-4-oxobutyl)carbamate (638)

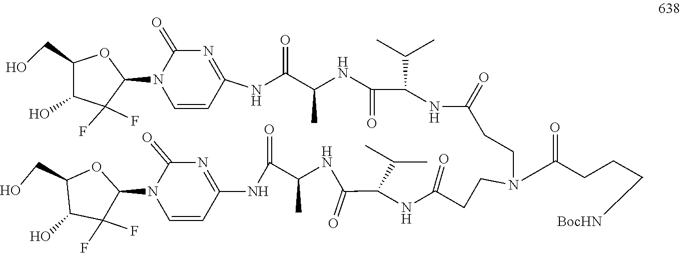 [SMILES: CC(C)[C@H](NC(=O)CCN(CCC(=O)N[C@H](C(=O)N[C@@H](C)C(=O)Nc1ccn([C@@H]2O[C@H](CO)[C@@H](O)C2(F)F)c(=O)n1)C(C)C)C(=O)CCCNC(=O)OC(C)(C)C)C(=O)N[C@@H](C)C(=O)Nc1ccn([C@@H]2O[C@H](CO)[C@@H](O)C2(F)F)c(=O)n1]

To a stirring solution of 616 (120.0 mg, 0.346 mmol) in DMF (5 mL) at room temperature were added 638 (413.47 mg, 0.954 mmol), HATU (395.19 mg, 1.039 mmol) and DIEA (179.11 mg, 1.386 mmol). The resulting mixture was stirred at room temperature for 15 min before it was concentrated in vacuo. HPLC reverse-phase purification (C18, d30×L250 cm, eluted with 5% CH₃CN in water to 70% CH₃CN in water at 15 ml/min in 50 min) and lyophilization afforded 638 (262.0 mg, 64%). HRMS (ESI): calcd for C₄₉H₇₃F₄N₁₂O₁₇ [M+H]⁺ 1177.18, found 1177.18.

### Example 438. Synthesis of 4-amino-N,N-bis(3-(((S)-1-(((S)-1-((1-((2R,4R,5R)-3,3-di fluoro-4-hydroxy-5-(hydroxymethyl)tetrahydrofuran-2-yl)-2-oxo-1,2-dihydropyrimidin-4-yl)amino)1-oxopropan-2-yl)amino)-3-methyl-1-oxobutan-2-yl)amino)-3-oxopropyl)butanamide (639)

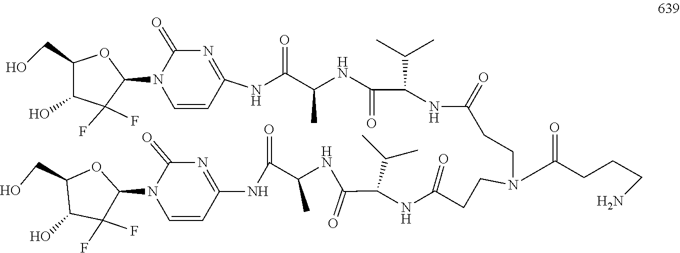 [SMILES: CC(C)[C@H](NC(=O)CCN(CCC(=O)N[C@H](C(=O)N[C@@H](C)C(=O)Nc1ccn([C@@H]2O[C@H](CO)[C@@H](O)C2(F)F)c(=O)n1)C(C)C)C(=O)CCCN)C(=O)N[C@@H](C)C(=O)Nc1ccn([C@@H]2O[C@H](CO)[C@@H](O)C2(F)F)c(=O)n1]

A stirring solution of 638 (100.0 mg, 0.085 mmol) in DCM (5 mL) was added TFA (1 mL) at room temperature. The resulting mixture was stirred at room temperature for 1 h before it was concentrated in vacuo. The product was triturated from MTBE to afforded 639 (90.0 mg, 100%). HRMS (ESI): calcd for C₄₄H₆₅F₄N₁₂O₁₅ [M+H]⁺ 1077.06, found 1077.06.

### Example 439. Synthesis of 4-((37S,45S,48S,51S)-45-((2S,5S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-10-(3-(((S)-1-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-3-methyl-1-oxobutan-2-yl)amino)-3-oxopropyl)-5-isopropyl-2-methyl-1,4,7,11,16-pentaoxo-3,6,10,15-tetraazaoctadecan-18-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (640)

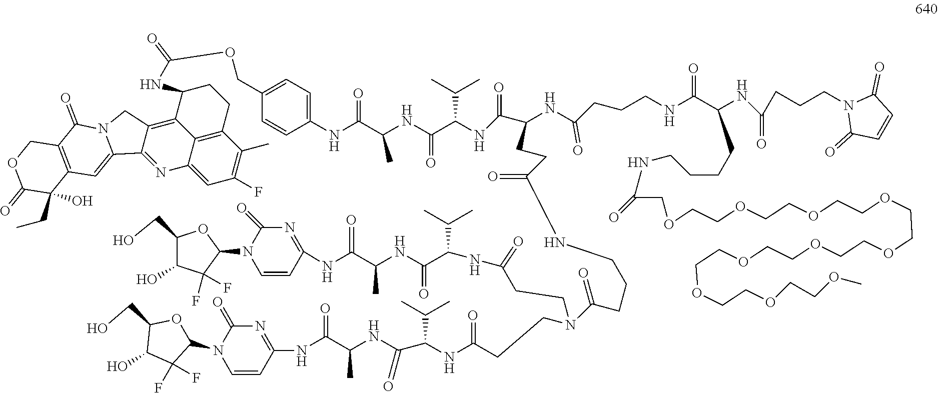

To a stirring solution of 639 (54 mg, 0.05 mmol) in DMF (4 ml) at room temperature were added 612 (87 mg, 0.05 mmol), HATU (23 mg, 0.06 mmol) and DIEA (10 mg, 0.08 mmol). The reaction mixture was stirred for 2.5 h at room temperature, and purified by pre-HPLC (C18, d30×L250 cm, eluted with 5% CH₃CN in water to 70% CH₃CN in water at 15 ml/min in 50 min) and lyophilized to afford 640 (44 mg, 31% yield) as a white solid. ESI MS m/z: [2M+H]⁺calcd for C128H₁₇₈F₅N₂₃O₄₁: found 1395.9.

### Example 440. Synthesis of compound (642)

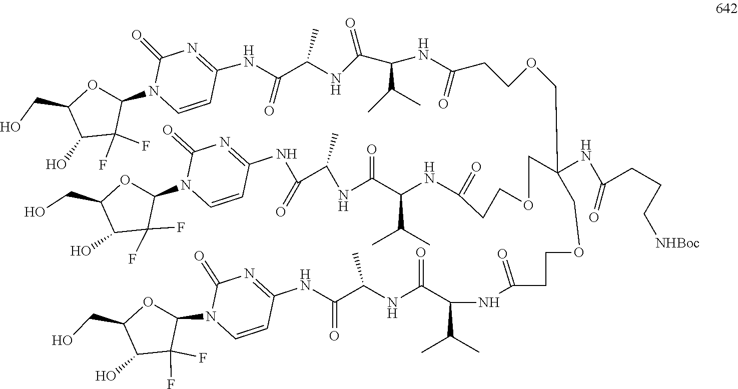 [SMILES: CC(C)[C@H](NC(=O)CCOCC(COCCC(=O)N[C@H](C(=O)N[C@@H](C)C(=O)Nc1ccn([C@@H]2O[C@H](CO)[C@@H](O)C2(F)F)c(=O)n1)C(C)C)(COCCC(=O)N[C@H](C(=O)N[C@@H](C)C(=O)Nc1ccn([C@@H]2O[C@H](CO)[C@@H](O)C2(F)F)c(=O)n1)C(C)C)NC(=O)CCCNC(=O)OC(C)(C)C)C(=O)N[C@@H](C)C(=O)Nc1ccn([C@@H]2O[C@H](CO)[C@@H](O)C2(F)F)c(=O)n1]

To a stirring solution of 594 (120.0 mg, 0.346 mmol) in DMF (5 mL) at room temperature were added 637 (599 mg, 1.384 mmol), HATU (526 mg, 1.384 mmol) and DIEA (179.11 mg, 1.386 mmol). The resulting mixture was stirred at room temperature for 15 min before it was concentrated in vacuo. HPLC reverse-phase purification (C18, d30×L250 cm, eluted with 5% CH₃CN in water to 70% CH₃CN in water at 15 ml/min in 50 min) and lyophilization afforded 642 (311 mg, yield 60%). ESI: m/z: calcd for C₇₃H₁₀₇F₆N₁₇O₂₇ [M+H]⁺: found 1769.8.

### Example 441. (2S,2'S)-2,2′-(((2S,5S)-12-(4-aminobutanamido)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-5-isopropyl-2-methyl-1,4,7-trioxo-12-((3-oxopropoxy)methyl)-10,14-dioxa-3,6-diazaheptadecan-17-oyl)bis(azanediyl))bis(N-((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)-3-methylbutanamide) (643)

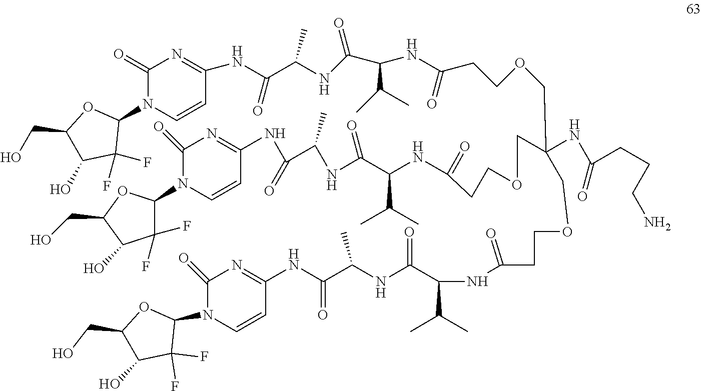 [SMILES: CC(C)[C@H](NC(=O)CCOCC(COCCC(=O)N[C@H](C(=O)N[C@@H](C)C(=O)Nc1ccn([C@@H]2O[C@H](CO)[C@@H](O)C2(F)F)c(=O)n1)C(C)C)(COCCC(=O)N[C@H](C(=O)N[C@@H](C)C(=O)Nc1ccn([C@@H]2O[C@H](CO)[C@@H](O)C2(F)F)c(=O)n1)C(C)C)NC(=O)CCCN)C(=O)N[C@@H](C)C(=O)Nc1ccn([C@@H]2O[C@H](CO)[C@@H](O)C2(F)F)c(=O)n1]

To a stirring solution of 642 (311 mg, 0.176 mmol) in DCM (15 mL) was added TFA (15 mL) at room temperature. The resulting mixture was stirred at room temperature for 0.5 h at 4° C. The mixture was concentrated in vacuo at 4° C. Suspension was obtained by adding EA (20 ml), filtered, to afford 643 (300 mg, 100%) as a white solid. ESI: m/z: calcd for C₆₈H₉₉F₆N₁₇O₂₅ [M+H]⁺: found 1669.8.

### Example 442. Synthesis of compound (644)

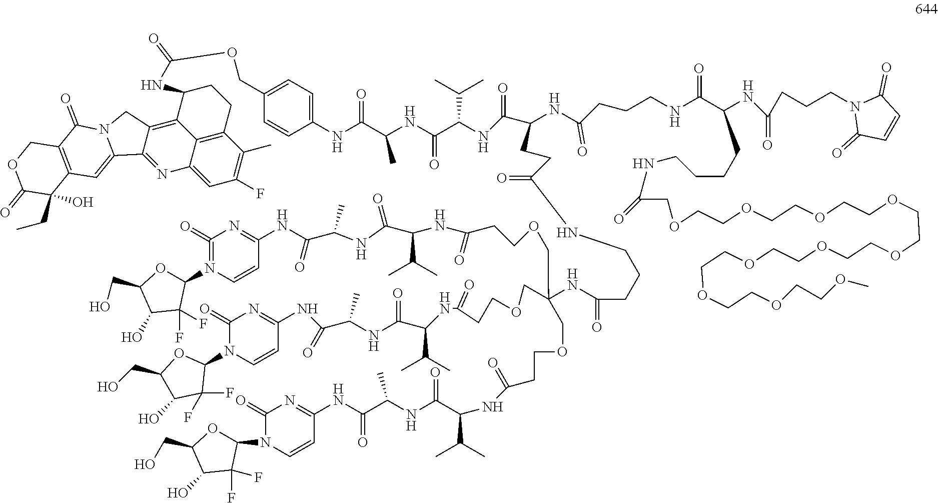

To a stirring solution of 643 (83 mg, 0.05 mmol) in DMF (4 ml) at room temperature were added 612 (87 mg, 0.05 mmol), HATU (23 mg, 0.06 mmol) and DIEA (10 mg, 0.08 mmol). The reaction mixture was stirred for 2.5 h at room temperature, and purified by pre-HPLC (C18, d30×L250 cm, eluted with 5% CH₃CN in water to 70% CH₃CN in water at 15 ml/min in 50 min) and lyophilized to afford 644 (83 mg, 50% yield) as a white solid. ESI MS m/z: [M+2H]²⁺ calcd for C₁₅₂H₂₁₃F₇N₂₈O₅₁: 1690.75, found 1691.28.

### Example 443. Synthesis of tert-butyl (4-((2-((1-((2R,4R,5R)-4-((tert-butoxycarbonyl)oxy)-5-(((tert-butoxycarbonyl)oxy)methyl)-3,3-difluorotetrahydrofuran-2-yl)-2-oxo-1,2-dihydropyrimidin-4-yl)amino)-2-oxoethyl)amino)-4-oxobutyl)carbamate (647)

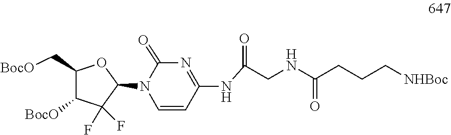

To a stirring solution of 623 (2 g, 4.3 mmol) in acetonitrile (30 ml) at 0° C. were added 626 (2.25 g, 8.6 mmol), TCFH (1.94 g, 6.9 mmol) and methylimidazole (1.06 g, 12.9 mmol). The reaction mixture was stirred for 3 h at room temperature, purified with S₁₀₂ column (DCM: EA=5:1) to afford 647 (1.52 g, 50% yield) as a white solid. ESI MS m/z: [M+H]⁺ calcd for C₃₀H₄₅F₂N₅O₁₂: found 706.30.

### Example 444. Synthesis of 4-amino-N-(2-((1-((2R,4R,5R)-3,3-difluoro-4-hydroxy-5-(hydroxy-methyl)tetrahydrofuran-2-yl)-2-oxo-1,2-dihydropyrimidin-4-yl)amino)-2-oxoethyl)butanamide (648)

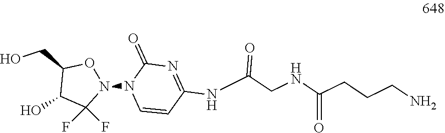

To a stirring solution of 647 (1.5 g, 2 mmol) in CH₂Cl₂ (5 ml) at room temperature was added TFA (5 ml), the reaction mixture was stirred for 0.5 h at room temperature, concentrated to afford 648 (O_{.86} g, 100% yield) as a colorless liquid. ESI MS m/z: [M+H]⁺calcd for C₁₅H₂₁F₂N₅O₆: found 406.15.

### Example 445. Synthesis of tert-butyl (1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-14,14-bis((3-((4-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-4-oxobutyl)amino)-3-oxopropoxy)methyl)-1,4,9,16-tetraoxo-12-oxa-3,8,15-triazanonadecan-19-yl)carbamate (649)

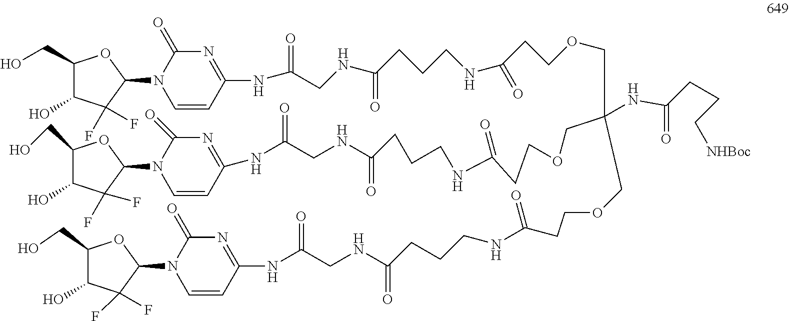 [SMILES: CC(C)(C)OC(=O)NCCCC(=O)NC(CCOCCC(=O)NCCCC(=O)NCC(=O)Nc1ccn([C@@H]2O[C@H](CO)[C@@H](O)C2(F)F)c(=O)n1)(COCCC(=O)NCCCC(=O)NCC(=O)Nc1ccn([C@@H]2O[C@H](CO)[C@@H](O)C2(F)F)c(=O)n1)COCCC(=O)NCCCC(=O)NCC(=O)Nc1ccn([C@@H]2O[C@H](CO)[C@@H](O)C2(F)F)c(=O)n1]

To a stirring solution of 648 (219 mg, 0.54 mmol) in DMF (2 ml) at room temperature were added 594 (94 mg, 0.18 mmol), HATU (342 mg, 0.90 mmol) and DIEA (140 mg, 1.08 mmol). The reaction mixture was stirred for 3 h at room temperature, purified by pre-HPLC (C18, d30×L250 cm, eluted with 5% CH₃CN in water to 70% CH₃CN in water at 15 ml/min in 50 min), and lyophilized to afford 649 (84 mg, 28% yield). ESI MS m/z: [M+H]⁺calcd for C₆₇H₉₅F₆N₁₇O₂₇: 1685.58; found 1685.58.

### Example 446. Synthesis of 4,4′-((14-(4-aminobutanamido)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1,4, 9-trioxo-14-((3-oxopropoxy)methyl)-12,16-dioxa-3,8-diazanonadecan-19-oyl)bis(azanediyl))bis(N-(2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)butanamide) (650)

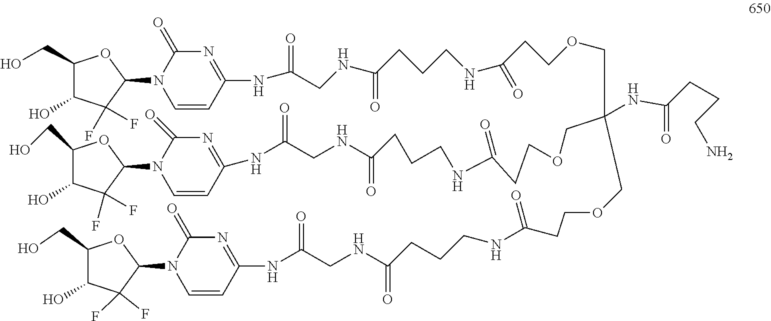 [SMILES: NCCCC(=O)NC(CCOCCC(=O)NCCCC(=O)NCC(=O)Nc1ccn([C@@H]2O[C@H](CO)[C@@H](O)C2(F)F)c(=O)n1)(COCCC(=O)NCCCC(=O)NCC(=O)Nc1ccn([C@@H]2O[C@H](CO)[C@@H](O)C2(F)F)c(=O)n1)COCCC(=O)NCCCC(=O)NCC(=O)Nc1ccn([C@@H]2O[C@H](CO)[C@@H](O)C2(F)F)c(=O)n1]

To a stirring solution of 649 (84 mg, 0.05 mmol) in CH₂Cl₂ (3 ml) at room temperature was added TFA (3 ml), the reaction mixture was stirred for 0.5 h at room temperature, concentrated to afford 650 (80 mg, 100% yield) as a colorless liquid. ESI MS m/z: [M+H]⁺calcd for C₆₂H₈₇F₆N₁₇O₂₅: 1585.46; found 1585.46.

### Example 447. Synthesis of 4-((37S,45S,48S,51S)-45-(1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-14,14-bis((3-((4-((2-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-oxoethyl)amino)-4-oxobutyl)amino)-3-oxopropoxy)methyl)-1,4,9,16,21-pentaoxo-12-oxa-3,8,15,20-tetraazatricosan-23-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (651)

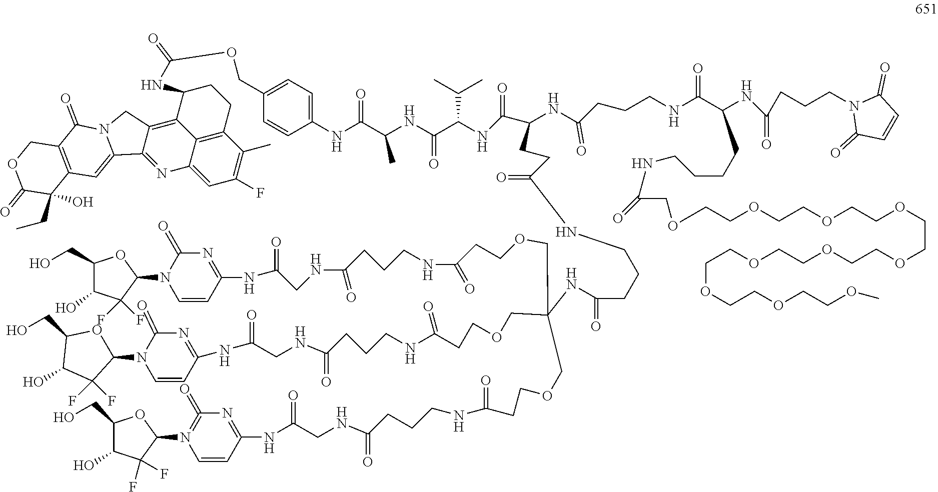

To a stirring solution of 650 (82 mg, 0.05 mmol) in DMF (4 ml) at room temperature were added 612 (87 mg, 0.05 mmol), HATU (23 mg, 0.06 mmol) and DIEA (10 mg, 0.08 mmol). The reaction mixture was stirred for 3.5 h at room temperature, and purified by pre-HPLC (C18, d30×L250 cm, eluted with 5% CH₃CN in water to 70% CH₃CN in water at 15 ml/min in 50 min), and lyophilized to afford 651 (49 mg, 29% yield) as a white solid. ESI MS m/z: [M+2H]²⁺ calcd for C₁₄₈H₂₀₁F₇N₂₈O₅₁: 1648.70, found 1648.70.

### Example 448. Synthesis of tert-butyl (2-((4-((1-((2R,4R,5R)-4-((tert-butoxycarbonyl)oxy)-5-(((tert-butoxycarbonyl)oxy)methyl)-3,3-difluorotetrahydrofuran-2-yl)-2-oxo-1,2-dihydropyrimidin-4-yl)amino)-4-oxobutyl)amino)-2-oxoethyl)carbamate (654)

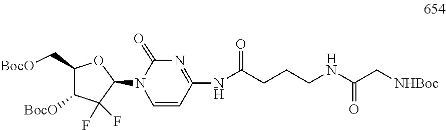

To a stirring solution of 623 (2.0 g, 4.3 mmol) in acetonitrile (30 ml) at 0° C. were added 653 (2.25 g, 8.6 mmol), TCFH (1.94 g, 6.9 mmol) and methylimidazole (1.06 g, 12.9 mmol). The reaction mixture was stirred for 3 h at room temperature, purified oxoethyl) carbamate with S₁₀₂ column (DCM: EA=5:1) to afford 754 (2.22 g, 72% yield) as a white solid. ESI MS m/z: [M+H]⁺calcd for C₃₀H₄₅F₂N₅O₁₂: found 706.27.

### Example 449. Synthesis of 4-(2-aminoacetamido)-N-(1-((2R,4R,5R)-3,3-difluoro-4-hydroxy-5-(hydroxymethyl)tetrahydrofuran-2-yl)-2-oxo-1,2-dihydropyrimidin-4-yl)butanamide (655)

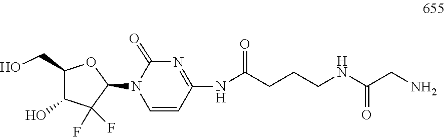 [SMILES: NCC(=O)NCCCC(=O)Nc1ccn([C@@H]2O[C@H](CO)[C@@H](O)C2(F)F)c(=O)n1]

To a stirring solution of 654 (1.41 g, 2 mmol) in CH₂Cl₂ (5 ml) at room temperature was added TFA (5 ml), the reaction mixture was stirred for 2 h at room temperature, concentrated to afford 655 (O_{.81} g, 100% yield) as a colorless liquid. ESI MS m/z: [M+H]⁺calcd for C₁₅H₂₁F₂N₅06: found 406.15.

### Example 450. Synthesis of tert-butyl (1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-14,14-bis((3-((2-((4-((1-((2R,4R,5R)-3,3-difluoro-4-hydroxy-5-(hydroxymethyl)tetrahydrofuran-2-yl)-2-oxo-1,2-dihydropyrimidin-4-yl)amino)-4-oxobutyl)amino)-2-oxoethyl)amino)-3-oxopropoxy)methyl)-1,6,9,16-tetraoxo-12-oxa-5,8,15-triazanonadecan-19-yl)carbamate (656)

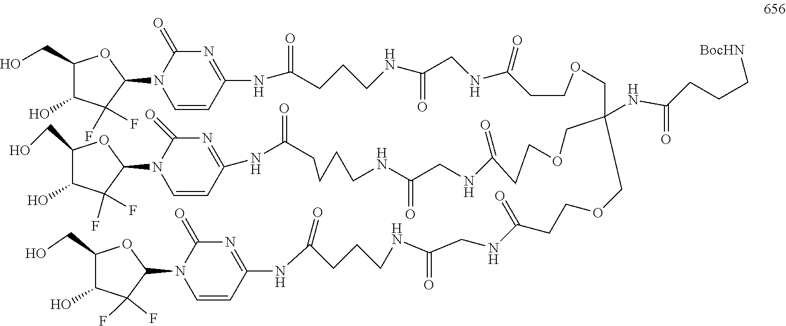

To a stirring solution of 655 (219 mg, 0.54 mmol) in DMF (2 ml) at room temperature were added 594 (94 mg, 0.18 mmol), HATU (342 mg, 0.90 mmol) and DIEA (140 mg, 1.08 mmol). The reaction mixture was stirred for 40 minutes at room temperature, purified by pre-HPLC (C18, d30 x L250 cm, eluted with 5% CH₃CN in water to 70% CH₃CN in water at 15 ml/min in 50 min), and lyophilized to afford 656 (240 mg, 56% yield). ESI MS m/z: [M+H]⁺ calcd for C₆₇H₉₅F₆N₁₇O₂₇: found 1685.64.

### Example 451. Synthesis of 4,4′-((9-(4-aminobutanamido)-9-((3-((2-((4-((1-((2R,4R,5R)-3,3-difluoro-4-hydroxy-5-(hydroxymethyl)tetrahydrofuran-2-yl)-2-oxo-1,2-dihydropyrimidin-4-yl)amino)-4-oxobutyl)amino)-2-oxoethyl)amino)-3-oxopropoxy)methyl)-4,14-dioxo-7,11-dioxa-3,15-diazaheptadecanedioyl)bis(azanediyl))bis(N-(1-((2R,4R,5R)-3,3-difluoro-4-hydroxy-5-(hydroxymethyl)tetrahydrofuran-2-yl)-2-oxo-1,2-dihydropyrimidin-4-yl)butanamide) (657)

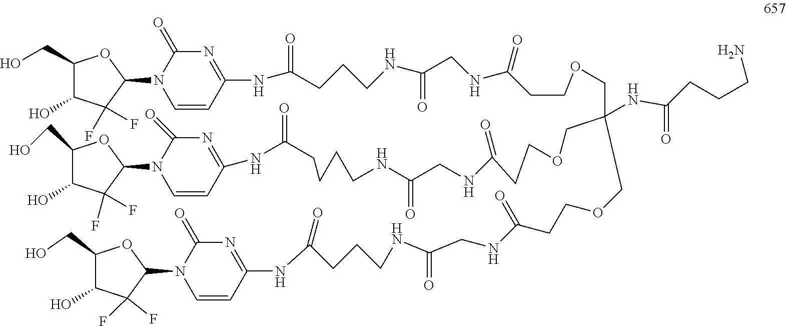

To a stirring solution of 656 (84 mg, 0.05 mmol) in CH₂Cl₂(3 ml) at room temperature was added TFA (3 ml), the reaction mixture was stirred for 0.5 h at room temperature, concentrated to afford 657 (80 mg, 100% yield) as a colorless liquid. ESI MS m/z: [M+H]⁺calcd for C₆₂H₈₇F₆N₁₇O₂₅: found 1585.54.

### Example 452. Synthesis of 4-((37S,45S,48S,51S)-45-(23-((1-((2R,4R,5R)-3,3-difluoro-4-hydroxy-5-(hydroxymethyl)tetrahydrofuran-2-yl)-2-oxo-1,2-dihydropyrimidin-4-yl)amino)-10,10-bis((3-((2-((4-((1-((2R,4R,5R)-3,3-difluoro-4-hydroxy-5-(hydroxymethyl)tetrahydrofuran-2-yl)-2-oxo-1,2-dihydropyrimidin-4-yl)amino)-4-oxobutyl)amino)-2-oxoethyl)amino)-3-oxopropoxy)methyl)-3,8,15,18,23-pentaoxo-12-oxa-4,9,16,19-tetraazatricosyl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (658)

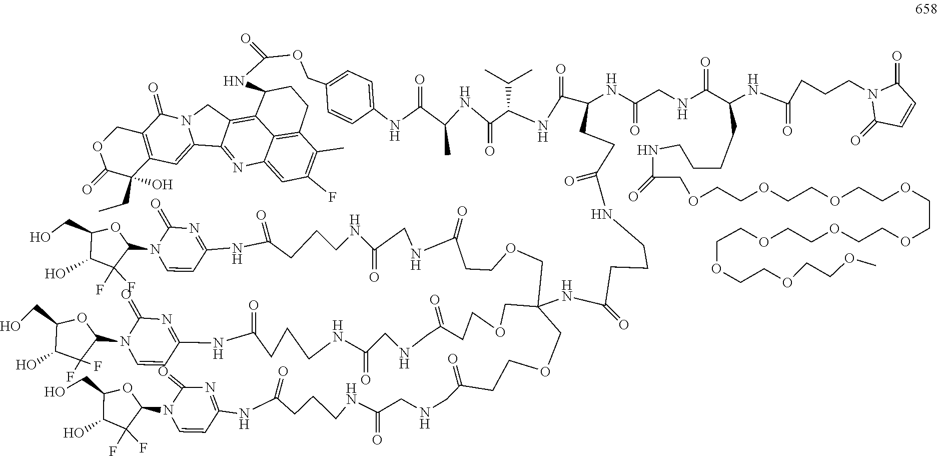

To a stirring solution of 657 (82 mg, 0.05 mmol) in DMF (4 ml) at room temperature were added 612 (87 mg, 0.05 mmol), HATU (23 mg, 0.06 mmol) and DIEA (10 mg, 0.08 mmol). The reaction mixture was stirred for 3.5 h at room temperature, and purified by pre-HPLC (C18, d30×L250 cm, eluted with 5% CH₃CN in water to 70% CH₃CN in water at 15 ml/min in 50 min), and lyophilized to afford 658 (86 mg, 52% yield) as a white solid. ESI MS m/z: [M+2H]²⁺ calcd for C₁₄₈H₂₀₁F₇N₂₈O₅₁: found 1648.6.

### Example 453. Synthesis of tert-butyl (4-(bis(3-((2-((4-((1-((2R,4R,5R)-3,3-difluoro-4-hydroxy-5-(hydroxymethyl)tetrahydrofuran-2-yl)-2-oxo-1,2-dihydropyrimidin-4-yl)amino)-4-oxobutyl)amino)-2-oxoethyl)amino)-3-oxopropyl)amino)-4-oxobutyl)carbamate (660)

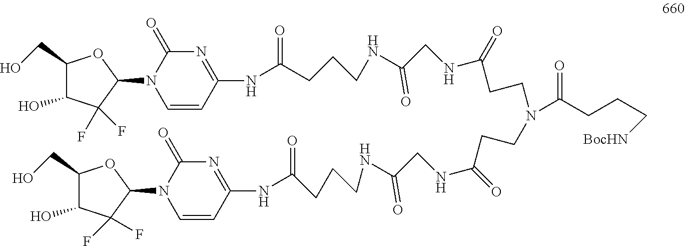 [SMILES: CC(C)(C)OC(=O)NCCCC(=O)N(CCC(=O)NCC(=O)NCCCC(=O)Nc1ccn([C@@H]2O[C@H](CO)[C@@H](O)C2(F)F)c(=O)n1)CCC(=O)NCC(=O)NCCCC(=O)Nc1ccn([C@@H]2O[C@H](CO)[C@@H](O)C2(F)F)c(=O)n1]

To a stirring solution of 616 (120 mg, 0.346 mmol) in DMF (5 mL) at room temperature were added 655 (387 mg, 0.954 mmol), HATU (395.19 mg, 1.039 mmol) and DIEA (179.11 mg, 1.386 mmol). The resulting mixture was stirred at room temperature for 3 h before it was concentrated in vacuo. HPLC reverse-phase purification (C18, d30×L250 cm, eluted with 5% CH₃CN in water to 70% CH₃CN in water at 15 ml/min in 50 min), and lyophilization afforded 80 (255.0 mg, 66%). HRMS (ESI): calcd for C₄₆H₆₄F₄N₁₂O₁₇ [M+H]⁺found 1122.1.

### Example 454. Synthesis of 4-amino-N,N-bis(3-((2-((4-((1-((2R,4R,5R)-3,3-difluoro-4-hydroxy-5-(hydroxymethyl)tetrahydrofuran-2-yl)-2-oxo-1,2-dihydropyrimidin-4-yl)amino)-4-oxobutyl)amino)-2-oxoethyl)amino)-3-oxopropyl)butanamide (661)

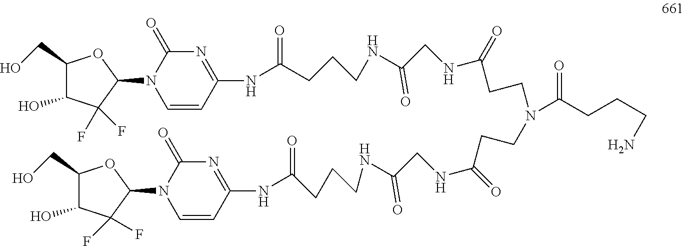 [SMILES: NCCCC(=O)N(CCC(=O)NCC(=O)NCCCC(=O)Nc1ccn([C@@H]2O[C@H](CO)[C@@H](O)C2(F)F)c(=O)n1)CCC(=O)NCC(=O)NCCCC(=O)Nc1ccn([C@@H]2O[C@H](CO)[C@@H](O)C2(F)F)c(=O)n1]

To a stirring solution of 660 (224 mg, 0.2 mmol) in CH₂Cl₂(5 ml) at room temperature was added TFA (5 ml), the reaction mixture was stirred for 0.5 h at room temperature, concentrated to afford 661 (205 mg, 100% yield) as a colorless liquid. ESI MS m/z: [M+H]⁺ calcd for C₄₀H₅₆F₄N₁₂O₁₅: found 1022.1.

### Example 455. Synthesis of 4-((37S,45S,48S,51S)-45-(20-((1-((2R,4R,5R)-3,3-difluoro-4-hydroxy-5-(hydroxymethyl)tetrahydrofuran-2-yl)-2-oxo-1,2-dihydropyrimidin-4-yl)amino)-9-(3-((2-((4-((1-((2R,4R,5R)-3,3-difluoro-4-hydroxy-5-(hydroxymethyl)tetrahydrofuran-2-yl)-2-oxo-1,2-dihydropyrimidin-4-yl)amino)-4-oxobutyl)amino)-2-oxoethyl)amino)-3-oxopropyl)-3,8,12,15,20-pentaoxo-4,9,13,16-tetraazaicosyl)-37-(4-(2,5-dioxo-2,5-dihydro-1H-pyrrol-1-yl) butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (662)

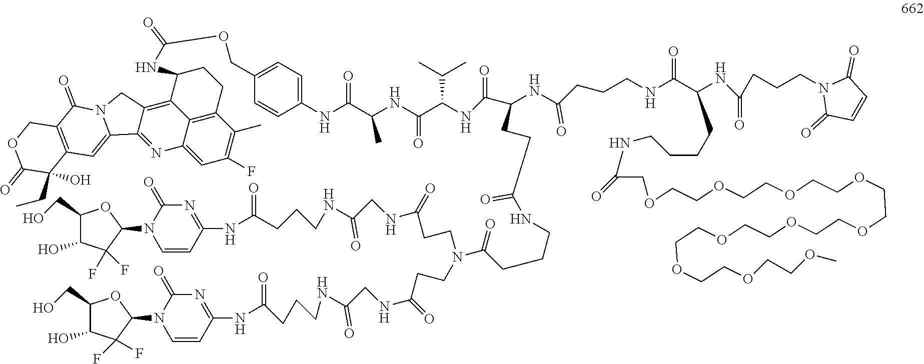 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)NCCCC(=O)N(CCC(=O)NCC(=O)NCCCC(=O)Nc2ccn([C@@H]4O[C@H](CO)[C@@H](O)C4(F)F)c(=O)n2)CCC(=O)NCC(=O)NCCCC(=O)Nc2ccn([C@@H]4O[C@H](CO)[C@@H](O)C4(F)F)c(=O)n2)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a stirring solution of 661 (51 mg, 0.05 mmol) in DMF (4 ml) at room temperature were added 612 (87 mg, 0.05 mmol), HATU (46 mg, 0.12 mmol) and DIEA (10 mg, 0.08 mmol). The reaction mixture was stirred for 2.5 h at room temperature, and purified by pre-HPLC (C18, d30×L250 cm, eluted with 5% CH₃CN in water to 70% CH₃CN in water at 15 ml/min in 50 min), and lyophilized to afford 662 (76 mg, 52% yield) as a white solid. ESI MS m/z: [M+2H]²⁺ calcd for C₁₂₄H₁₇₀F₅N₂₃O₄₁: 1367.1, found 1366.9.

### Example 456. Synthesis of tert-butyl ((S)-1-(((S)-1-((2-hydroxyethyl)amino)-1-oxopropan-2-yl)amino)-3-methyl-1-oxobutan-2-yl)carbamate (665)

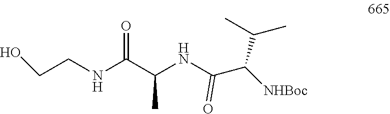 [SMILES: CC(C)[C@H](NC(=O)OC(C)(C)C)C(=O)N[C@@H](C)C(=O)NCCO]

To a stirred solution of 635 (3.25 g, 11.0 mmol) in DCM (150 mL) at room temperature were added 84 (O_{.69} g, 11.0 mmol), HATU (4.71 g, 12 mmol) and DIPEA (2.91 g, 23 mmol). The reaction mixture was stirred for 3 h at room temperature, washed with water (200 ml), brine (200 ml), dried over anhydrous Na₂SO₄, filtered and concentrated in vacuo to afford 665 (3.74 g, 100% yield) as a colorless liquid. ESI MS m/z: [M+H]⁺calcd for C₁₉H₂₉N₃O₅: found 332.5.

### Example 457. Synthesis of tert-butyl ((S)-3-methyl-1-(((S)-1-((2-(((4-nitrophenoxy) carbonyl)oxy)ethyl)amino)-1-oxopropan-2-yl)amino)-1-oxobutan-2-yl)carbamate 666

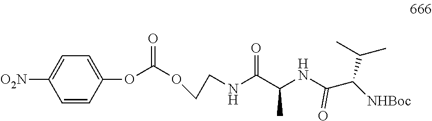 [SMILES: CC(C)[C@H](NC(=O)OC(C)(C)C)C(=O)N[C@@H](C)C(=O)NCCOC(=O)Oc1ccc([N+](=O)[O-])cc1]

To a solution of 665 (3.65 g, 11 mmol) and 687 (4-nitrophenyl carbonochloridate) (5.54 g, 27 mmol) in THF (100 mL) was added pyridine (4.35 g, 55 mmol) at r.t. The mixture was stirred at RT for 3 h. The reaction mixture was extracted with DCM (20 mL×3) and H₂O (20 mL). The organic layers were washed with 10% citric acid solution (20 mL×2), dried over anhydrous Na₂SO₄ and concentrated in vacuo. The residue was purified by column chromatography on silica gel (eluted with PE/EA=1/1) to afford 666 (3.30 g, 61% yield) as a white solid.

### Example 458. Synthesis of tert-butyl ((S)-1-(((S)-1-((2-(((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)ethyl)amino)-1-oxopropan-2-yl)amino)-3-methyl-1-oxobutan-2-yl)carbamate (667)

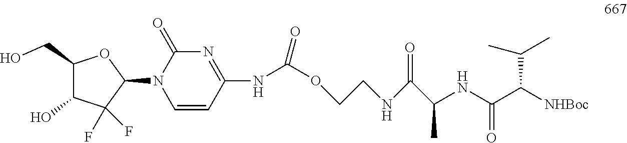 [SMILES: CC(C)[C@H](NC(=O)OC(C)(C)C)C(=O)N[C@@H](C)C(=O)NCCOC(=O)Nc1ccn([C@@H]2O[C@H](CO)[C@@H](O)C2(F)F)c(=O)n1]

To a solution of 666 (496 mg, 1.00 mmol) and 9 (263 mg, 1.00 mmol) in DMF (30 mL) was added DIPEA (387 mg, 3.00 mmol) at r.t. The mixture was stirred at 65° C. overnight. The reaction mixture was concentrated in vacuo. The residue was purified by column chromatography on silica gel (eluted with DCM/MeOH=15/1~10/1) to afford 667 (434 mg, 70% yield) as a white solid. ESI MS m/z: [M+H]⁺ calcd for C₂₅H₃₈F₂N₆O₁₀: found 621.6.

### Example 459. Synthesis of 2-((S)-2-((S)-2-amino-3-methylbutanamido)propanamido)ethyl (1-((2R,4R,5R)-3,3-difluoro-4-hydroxy-5-(hydroxymethyl)tetrahydrofuran-2-yl)-2-oxo-1,2-dihydropyrimidin-4-yl)carbamate (668)

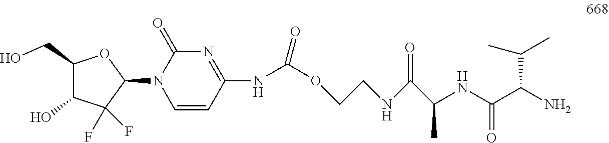 [SMILES: CC(C)[C@H](N)C(=O)N[C@@H](C)C(=O)NCCOC(=O)Nc1ccn([C@@H]2O[C@H](CO)[C@@H](O)C2(F)F)c(=O)n1]

To a stirring solution of 667 (1.24 g, 2.00 mmol) in CH₂Cl₂(20 ml) at room temperature was added TFA (20 ml), the reaction mixture was stirred for 0.5 h, concentrated to afford 668 (1.05 g, 100% yield) as a colorless liquid. ESI MS m/z: [M+H]⁺calcd for C₂₀H₃₀F₂N₆O₈: found 521.50.

### Example 460. Synthesis of (5S,8S,18S,21S)-13-(4-((tert-butoxycarbonyl)amino)butanoyl)-8,18-diisopropyl-5,21-dimethyl-4,7,10,16,19,22-hexaoxo-3,6,9,13,17,20,23-heptaazapentacosane-1,25-diyl bis((1-((2R,4R,5R)-3,3-difluoro-4-hydroxy-5-(hydroxymethyl)tetrahydrofuran-2-yl)-2-oxo-1,2-dihydropyrimidin-4-yl)carbamate) (669)

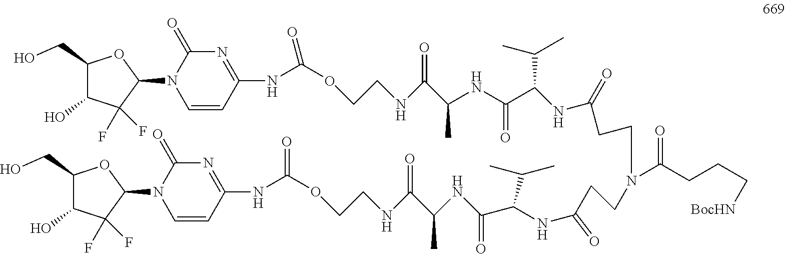 [SMILES: CC(C)[C@H](NC(=O)CCN(CCC(=O)N[C@H](C(=O)N[C@@H](C)C(=O)NCCOC(=O)Nc1ccn([C@@H]2O[C@H](CO)[C@@H](O)C2(F)F)c(=O)n1)C(C)C)C(=O)CCCNC(=O)OC(C)(C)C)C(=O)N[C@@H](C)C(=O)NCCOC(=O)Nc1ccn([C@@H]2O[C@H](CO)[C@@H](O)C2(F)F)c(=O)n1]

To a stirring solution of 616 (120 mg, 0.346 mmol) in DMF (5 mL) at room temperature were added 655 (496 mg, 0.954 mmol), HATU (395.19 mg, 1.039 mmol) and DIEA (179.11 mg, 1.386 mmol). The resulting mixture was stirred at room temperature for 3 h before it was concentrated in vacuo. HPLC reverse-phase purification (C18, d30×L250 cm, eluted with 5% CH₃CN in water to 70% CH₃CN in water at 15 ml/min in 50 min), and lyophilization afforded 669 (301.0 mg, 64%). HRMS (ESI): calcd for C₅₅H₈₂F₄N₁₄O₂₁ [M+H]⁺found 1352.4.

### Example 461. Synthesis of (5S,8S,18S,21S)-13-(4-aminobutanoyl)-8,18-diisopropyl-5,21-dimethyl-4,7,10,16,19,22-hexaoxo-3,6,9,13,17,20,23-heptaazapentacosane-1,25-diyl bis((1-((2R,4R,5R)-3,3-difluoro-4-hydroxy-5-(hydroxymethyl)tetrahydrofuran-2-yl)-2-oxo-1,2-dihydropyrimidin-4-yl)carbamate) (670)

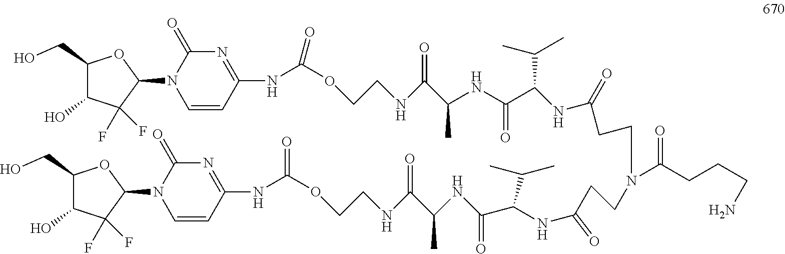 [SMILES: CC(C)[C@H](NC(=O)CCN(CCC(=O)N[C@H](C(=O)N[C@@H](C)C(=O)NCCOC(=O)Nc1ccn([C@@H]2O[C@H](CO)[C@@H](O)C2(F)F)c(=O)n1)C(C)C)C(=O)CCCN)C(=O)N[C@@H](C)C(=O)NCCOC(=O)Nc1ccn([C@@H]2O[C@H](CO)[C@@H](O)C2(F)F)c(=O)n1]

To a stirring solution of 669 (135 mg, 0.10 mmol) in CH₂C₁₂ (5 ml) at room temperature was added TFA (5 ml), the reaction mixture was stirred for 0.5 h at room temperature, concentrated to afford 670 (125 mg, 100% yield) as a colorless liquid. ESI MS m/z: [M+H]⁺ calcd for C₅₀H₇₄F₄N₁₄O₁₉: found 1252.3.

### Example 462. Synthesis of compound (671)

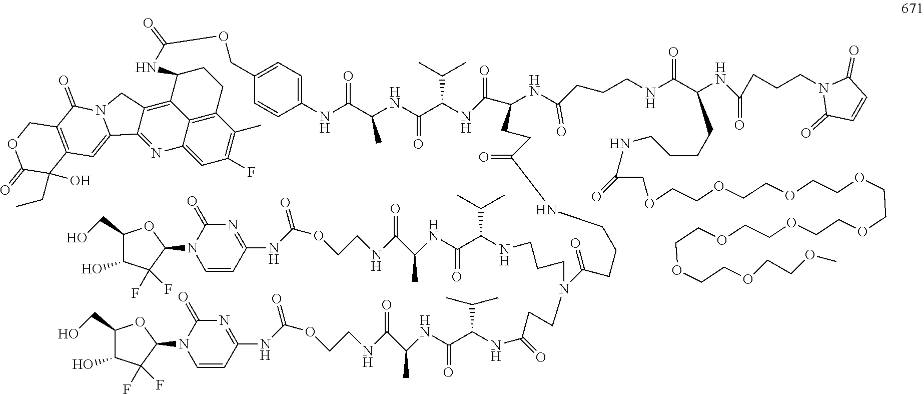 [SMILES: CCC1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)NCCCC(=O)N(CCCN[C@H](C(=O)N[C@@H](C)C(=O)NCCOC(=O)Nc2ccn([C@@H]4O[C@H](CO)[C@@H](O)C4(F)F)c(=O)n2)C(C)C)CCC(=O)N[C@H](C(=O)N[C@@H](C)C(=O)NCCOC(=O)Nc2ccn([C@@H]4O[C@H](CO)[C@@H](O)C4(F)F)c(=O)n2)C(C)C)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a stirring solution of 670 (63 mg, 0.05 mmol) in DMF (4 ml) at room temperature were added 612 (87 mg, 0.05 mmol), HATU (23 mg, 0.06 mmol) and DIEA (10 mg, 0.08 mmol). The reaction mixture was stirred for 2.5 h at room temperature, and purified by pre-HPLC (C18, d30×L250 cm, eluted with 5% CH₃CN in water to 70% CH₃CN in water at 15 ml/min in 50 min), and lyophilized to afford 671 (88 mg, 60% yield) as a white solid. ESI MS m/z: [2M+H]⁺ calcd for C₁₃₄H₁₈₈F₅N₂₅O₄₅: found 1483.1.

### Example 463. Synthesis of compound (673)

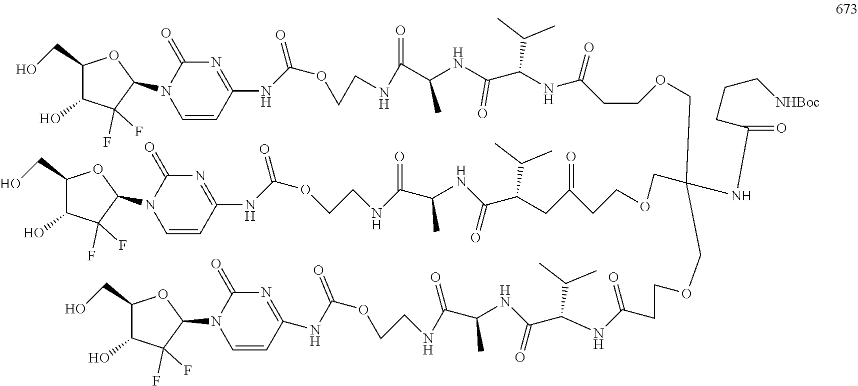 [SMILES: CC(C)[C@H](CC(=O)CCOCC(COCCC(=O)N[C@H](C(=O)N[C@@H](C)C(=O)NCCOC(=O)Nc1ccn([C@@H]2O[C@H](CO)[C@@H](O)C2(F)F)c(=O)n1)C(C)C)(COCCC(=O)N[C@H](C(=O)N[C@@H](C)C(=O)NCCOC(=O)Nc1ccn([C@@H]2O[C@H](CO)[C@@H](O)C2(F)F)c(=O)n1)C(C)C)NC(=O)CCCNC(=O)OC(C)(C)C)C(=O)N[C@@H](C)C(=O)NCCOC(=O)Nc1ccn([C@@H]2O[C@H](CO)[C@@H](O)C2(F)F)c(=O)n1]

To a stirring solution of 668 (260 mg, 0.54 mmol) in DMF (2 ml) at room temperature were added 594 (94 mg, 0.18 mmol), HATU (342 mg, 0.90 mmol) and DIEA (140 mg, 1.08 mmol). The reaction mixture was stirred for 3 h at room temperature, purified by pre-HPLC (C18, d30×L250 cm, eluted with 5% CH₃CN in water to 70% CH₃CN in water at 15 ml/min in 50 min), and lyophilized to afford 673 (210 mg, 57% yield). ESI MS m/z: [M+H]⁺ calcd for C₈₂H₁₂2F₆N₂₀O₃₃: found 2031.1.

### Example 464. Synthesis of compound (674)

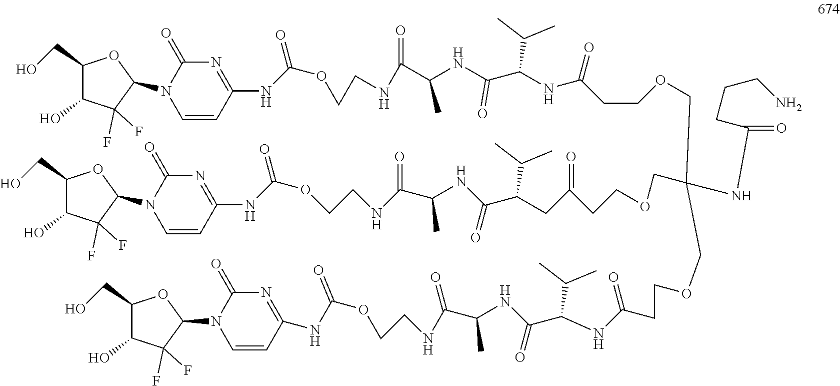 [SMILES: CC(C)[C@H](CC(=O)CCOCC(COCCC(=O)N[C@H](C(=O)N[C@@H](C)C(=O)NCCOC(=O)Nc1ccn([C@@H]2O[C@H](CO)[C@@H](O)C2(F)F)c(=O)n1)C(C)C)(COCCC(=O)N[C@H](C(=O)N[C@@H](C)C(=O)NCCOC(=O)Nc1ccn([C@@H]2O[C@H](CO)[C@@H](O)C2(F)F)c(=O)n1)C(C)C)NC(=O)CCCN)C(=O)N[C@@H](C)C(=O)NCCOC(=O)Nc1ccn([C@@H]2O[C@H](CO)[C@@H](O)C2(F)F)c(=O)n1]

To a stirring solution of 673 (203 mg, 0.10 mmol) in CH₂C₁₂ (5 ml) at room temperature was added TFA (5 ml), the reaction mixture was stirred for 0.5 h, concentrated to afford 674 (193 mg, 100% yield) as a colorless liquid. ESI MS m/z: [M+H]⁺ calcd for C₇₇H₁₁₄F₆N₂₀O₃₁: found 1930.9.

### Example 465. Synthesis of compound (675)

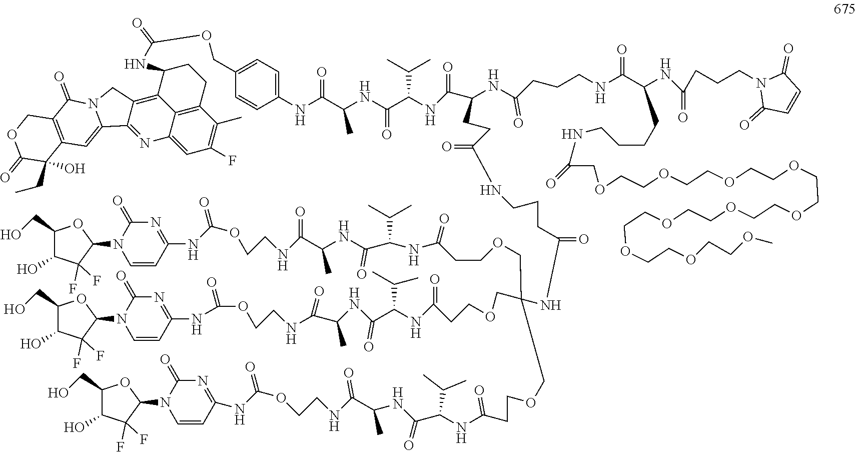

To a stirring solution of 670 (97 mg, 0.05 mmol) in DMF (4 ml) at room temperature were added 612 (87 mg, 0.05 mmol), HATU (46 mg, 0.12 mmol) and DIEA (10 mg, 0.08 mmol). The reaction mixture was stirred for 3.5 h at room temperature, and purified by pre-HPLC (C18, d30×L250 cm, eluted with 5% CH₃CN in water to 70% CH₃CN in water at 15 ml/min in 50 min), and lyophilized to afford 675 (132 mg, 60% yield) as a white solid. ESI MS m/z: [M+2H]²⁺+calcd for C₁₆₁H₂₂₈F₇N₃₁O₅₇: found 1822.4.

### Example 466. Synthesis of tert-butyl (4-((1,3-bis(3-((1-((2R,4R,5R)-4-((tert-butoxy carbonyl)oxy)-5-(((tert-butoxycarbonyl)oxy)methyl)-3,3-difluorotetrahydrofuran-2-yl)-2-oxo-1,2-dihydropyrimidin-4-yl)amino)-3-oxopropoxy)-2-((3-((1-((2R,4R,5R)-4-((tert-butoxycarbonyl)oxy)-5-(((tert-butoxycarbonyl)oxy)methyl)-3,3-difluorotetrahydrofuran-2-yl)-2-oxo-1,2-dihydro pyrimidin-4-yl)amino)-3-oxopropoxy)methyl)propan-2-yl)amino)-4-oxobutyl)carbamate (677)

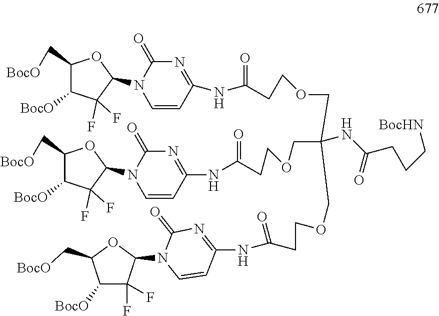

To a stirring solution of 668 (250 mg, 0.54 mmol) in DMF (2 ml) at room temperature were added 594 (94 mg, 0.18 mmol), HATU (342 mg, 0.90 mmol) and DIEA (140 mg, 1.08 mmol). The reaction mixture was stirred for 3 h at room temperature, purified by pre-HPLC (C18, d30×L250 cm, eluted with 5% CH₃CN in water to 70% CH₃CN in water at 15 ml/min in 50 min), and lyophilized to afford 677 (198 mg, 59% yield). ESI MS m/z: [M+H]⁺ calcd for C₇₉H₁₁₃F₆N₁₁O₃₃: found 1859.9.

### Example 467. Synthesis of 3,3′-((2-(4-aminobutanamido)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)propane-1,3-diyl)bis(oxy))bis(N-(1-((2R,4R,5R)-3,3-difluoro-4-hydroxy-5-(hydroxymethyl)tetrahydrofuran-2-yl)-2-oxo-1,2-dihydropyrimidin-4-yl)propanamide) (678)

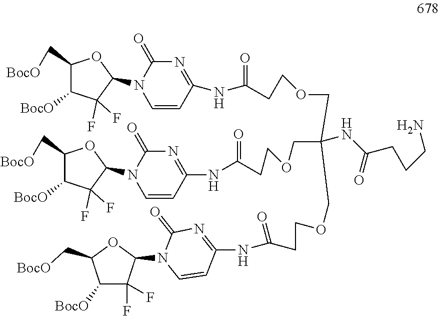

To a stirring solution of 677 (186 mg, 0.10 mmol) in CH₂C₁₂ (5 ml) at room temperature was added TFA (5 ml), the reaction mixture was stirred for 0.5 h at room temperature, concentrated to afford 678 (116 mg, 100% yield) as a colorless liquid. ESI MS m/z: [M+H]⁺calcd for C₄₄H₅₇F₆N₁₁O₁₉: found 1159.0.

### Example 468. Synthesis of 4-((37S,45S,48S,51S)-45-(3-((4-((1,3-bis(3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-2-((3-((1-((2R,4R,5R)-3,3-difluoro-4-hydroxy-5-(hydroxymethyl)tetra hydrofuran-2-yl)-2-oxo-1,2-dihydropyrimidin-4-yl)amino)-3-oxopropoxy)methyl)propan-2-yl)amino)-4-oxobutyl)amino)-3-oxopropyl)-37-(4-(2,5-dioxo-2,5-dihydro-1H-pyrrol-1-yl) butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (679)

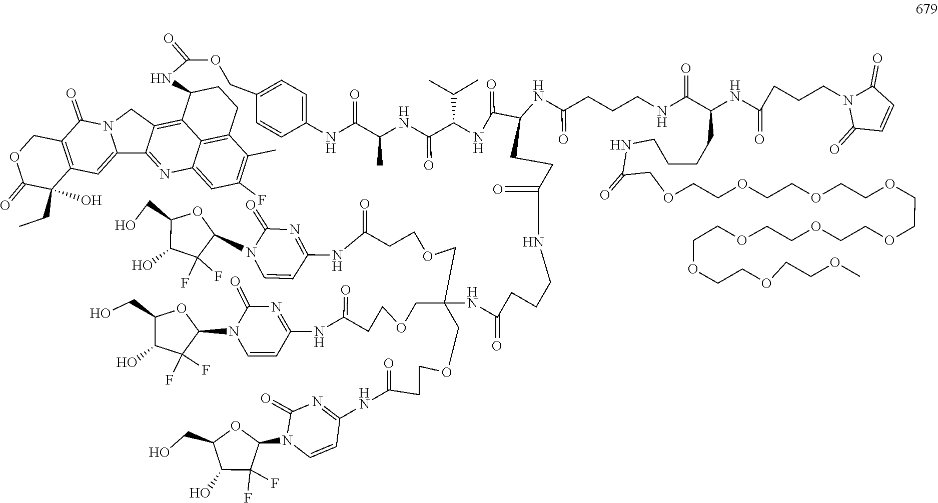 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)NCCCC(=O)NC(COCCC(=O)Nc2ccn([C@@H]4O[C@H](CO)[C@@H](O)C4(F)F)c(=O)n2)(COCCC(=O)Nc2ccn([C@@H]4O[C@H](CO)[C@@H](O)C4(F)F)c(=O)n2)COCCC(=O)Nc2ccn([C@@H]4O[C@H](CO)[C@@H](O)C4(F)F)c(=O)n2)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1)CC3]

To a stirring solution of 678 (58 mg, 0.05 mmol) in DMF (4 ml) at room temperature were added 612 (87 mg, 0.05 mmol), HATU (46 mg, 0.12 mmol) and DIEA (10 mg, 0.08 mmol). The reaction mixture was stirred for 3.5 h at room temperature, and purified by pre-HPLC (C18, d30×L250 cm, eluted with 5% CH₃CN in water to 70% CH₃CN in water at 15 ml/min in 50 min), and lyophilized to afford 679 (82 mg, 56% yield) as a white solid. ESI MS m/z: [M+2H]² calcd for C₁₂₈H₁₇₁F₇N₂₂O₄₅: found 1436.5.

### Example 469. A traditional method of producing an antibody-drug conjugate (ADC) comprising a monoclonal antibody conjugated to a cytotoxin having a terminal of maleimido group

A monoclonal antibody was conjugated to a cytotoxin having a terminal of maleimido group. Specifically, purified antibody was incubated with a 2.0-12.0 molar excess of the reducing agent TCEP (Tris(2-carboxyethyl)phosphine) in PBS pH 6.2~7.5, 1 mM EDTA (Ethylenediamine tetraacetic acid) for 1 hours at 37° C. Subsequently, 5.0-12.0 equivalents of the payload of a cytotoxin having a terminal of maleimido group from a stock solution in 10% (v/v) DMA or DMSO was added, followed by incubation at room temperature for one hour to 3 hours under gentle rotation. The conjugation reaction was optionally quenched by the addition of four molar equivalents (over the payload) of N-acetyl cysteine, and the TCEP was optionally quenched by the addition of 1 or 2 molar equivalents of 4-azidomethylbenzoic acid or 4-azidobenzoic acid. After incubation, the reducing agent and the excess payload/linker complexes were removed by 2-10 times of dialysis in PBS pH 5.0-7.2, at 4° C. using 20,000 MWCO dialysis cassettes or purified by ion exchange chromatography. For the payload/linker complex containing a disulfide bond, the reduced antibody was isolated through a chromatography (with ion exchange or size exclusion column) or dialysis prior to run conjugation reaction. For PBD-ADCs that contain imine group was preferably added Na₂SO₃ (2~50 eq. of the payload used in the conjugation (prior to the step of purification) so that the imine group of the conjugation can be converted to the sulfonate prodrug. For Dxd-GGFG conjugation, the conjugates with DAR >7.2 were purified against formulated buffers (normally, 0.02% Tween-20 or −80, 6~7% sugar, 20~50 mM histine, pH=5.5~7.0).

The conjugation process may result in 0.1 to 10% of aggregate formation. Thus, macromolecular aggregates, conjugation reagents, including payloads quenched by cysteine, and other added regents can be removed using ceramic hydroxyapatite Type II chromatography (CHT) as described e.g. Thompson et al., J. Control Release, 236: 100-116 (2016) or by ion exchange chromatography. The ADCs were optionally formulated in 25 mM Histidine-HCl, or citrate buffer containing, 6~7% sucrose, 0.02% polysorbate-20 or 80, and 0.10% methionine, at pH 5.0-6.5.

### Example 470. Preparation of ADC conjugates of the present invention via the homogeneous conjugation reaction

A zinc amino complex (e.g. Zinc 2-methylpropane-1, 2-diamine chloride complex) (in 10-60 mM, 1.0-5.0 eq. of an antibody used) and TCEP (in 100 mM, 2.5-7.5 eq. of an antibody used) were added in sequence to a solution containing the antibody (10-50 mg/mL, in 20 mM PBS, pH 5.5-7.5) at 2-8° C. After incubation at 2-8° C. for 12-20 h (overnight), a payload/linker complex (100-200 mM, 2.0-15.0 eq. of the antibody used) was introduced and the incubation was continued for further 2-4 h at 2-8° C. After the incubation, cystine or 4-(azidomethyl)benzoic acid or 4-azidobenzoic acid (100-200 mM, 4.0-10.0 eq. of the antibody) was added to deplete the excess TCEP, cysteine (100-200 mM, 2.0-10.0 eq. of the antibody) was added to deplete the excess payload/linker complex, EDTA (100-200 mM, 4.0-6.0 eq. of the antibody) was added to trap zinc, and DHAA (100-200 mM, 8.0-30.0 eq. of the antibody) was added to oxidize (re-bridge linking) the free thiol groups in the antibody. The reaction mixture was finally purified using a de-salting column (Zeba Spin Desalting Columns, 40K MWCO), or UF/DF, or ion exchange chromatography, and drug/antibody ratio (DAR) were analyzed using HIC-HPLC or HPLC-MS. For the payload/linker complex containing a disulfide bond, the reduced antibody was isolated at 2-8° C. through a chromatography (with ion exchange or size exclusion column) or dialysis prior to run conjugation reaction at 2-8° C. For PBD-ADCs that contain imine group was preferably added Na₂SO₃ (2-50 eq. of the payload used in the conjugation (prior to the step of purification) so that the imine group of the conjugation can be converted to the sulfonate prodrug.

### Example 471. Preparation of more stable ADC conjugates having a maleimide terminal, particularly having dual-maleimides vis the thioether linkage

Since cytotoxic agents in cysteine-linked ADCs can be released via thiol-maleimide exchange in plasma (Ponte, F., et al, Bioconjug Chem. 2016, 27(7), 1588-98, Fontaine, S., et al, Bioconjug Chem. 2015, 26(1): 145-52) and may show toxicities. Thus, a maleimide-thiol conjugate is preferably hydrolyzed to form a ring-opened moiety, the 4-amino-4-oxobutanoic acid product, which is much more stabilized toward cleavage (Fontaine, S., et al, Bioconjug Chem. 2015, 26(1): 145-52; Zheng, K., et al, J Pharm Sci. 2019, 108(1):133-141). Therefore, when the conjugation reaction is finished prior to the purification step, the reaction mixture was adjusted to pH 7.0-8.5, with optionally addition of tween-20 or tween-80 to reach concentration of 0.01%~0.5%, and then the mixture was gently stirred at 15-40° C. for 4-96 h. Once the hydrolysis of the succinimide ring was confirmed by LC-MS/MS, then the purification of the ADC was proceeded as described above.

### Example 472. Characterization of the antibody and the ADC conjugate

To determine monomeric content, aggregates, and fragments of ADCs, analytical size-exclusion chromatography (SEC-HPLC) was performed using 100 m (100 μL volume) of antibodies or ADCs, which were loaded into a TSKgel.RTM. G3000WXL column (Tosoh Bioscience, Tokyo, Japan). The mobile phase was composed of 0.1 M sodium sulfate, 0.1 M sodium phosphate, and 10% isopropanol, pH 6.0~7.0. The flow rate was 1 mL/min, and each analysis was carried out for 10-45 minutes at room temperature. Hydrophobic interaction chromatography (HIC-HPLC) was used to assess conjugation and drug load distribution, and was performed using a butyl-non porous resin (NPR) column (4.6 mm ID×3.5 cm, 2.5 pm, Tosoh Bioscience). The mobile phase A was composed of 25 mM Tris-HCl, 1.5 M sulfate, pH 7.0~8.5; and the mobile phase B was composed of 25 mM Tris-HCl and 5% isopropanol, pH 8.0. 100 μL of antibodies or ADCs at a concentration of I mg/mL were loaded and eluted at a flow rate of 1 mL/min with a gradient of 5% B to 100% B over 10-30 min. Reduced reverse phase chromatography (rRP-HPLC) was used to confirm chain-specific conjugation. The antibodies and ADCs were reduced at 37° C. for 20 minutes using 42 mM dithiothreitol (DTT) in PBS (pH 7.2). 10 μg of reduced antibodies or ADCs were loaded onto a polymeric reverse phase media (PLRP-S) 1000 A column (2.1×50 mm) (Agilent Technologies, Santa Clara, Calif.) and eluted at a flow rate of 1 mL/min with a gradient of 5% B to 100% B over 20-35 minutes (mobile phase A: 0.1% trifluoroacetic acid in water; mobile phase B: 0.1% trifluoroacetic acid in acetonitrile).

Conjugation at the heavy and light chains and drug/antibody ratios (DAR) were determined by reduced liquid chromatography mass spectrometry analysis (rLC-MS) performed on an Agilent 1290 series uHPLC coupled to an Agilent 6230 TOF (Agilent Technologies, Santa Clara, Calif.). 2 μg of reduced antibodies or ADCs were loaded onto a ZORBAX.RTM. rapid resolution high definition (RRHD) 300-Diphenyl column (2.1×50 mm, 1.8 μm) (Agilent Technologies, Santa Clara, Calif.) and eluted at a flow rate of 0.5 mL/min using a step gradient of 80% B after 2.1 min (mobile phase A: 0.10% Formic acid in water and mobile phase B: 0.10% Formic acid in acetonitrile). A positive time-of-flight MS scan was acquired, and data collection and processing were carried out using MassHunter software (Agilent Technologies, Santa Clara, Calif.).

### Example 473. Cell Binding Assay by Flow cytometry

To detect the binding affinity, 5×10⁴ cell/well target cells were seed in 96-well plate (U-shaped bottom). Then the target cells were incubated with ADC for I h at 4° C. Human IgGI was set as isotype control. After incubation, cells were washed twice with PBS containing 2% FBS. The cells were then incubated with Goat Anti-Human IgG Fc Antibody, Fluorescein (FITC) Conjugate at 1:1000 dilution for 30 min at 4° C. After incubation, cells were washed three times with PBS containing 2% FBS. Flow cytometry was used to detect the Medians Fluorescence Intensity by different concentrations of an ADC to cell surface antigens. Kd values were calculated by a software, such as Prism or Excel software.

### Example 474. The methods of killing prostate cancer cells in vitro using the antibody-drug conjugates

Killing of prostate cancer cell and other solid tumor cell lines by antibody-drug conjugates of this invention, was evaluated in vitro using the protocol recommended, such as in the CCK8 kit (Dojindo Laboratories, Japan). Briefly, 5000 cells in 180 μL RPMI+10% FBS were added to the inner wells of 96-well plates. The following target-expressing cell lines were tested. The antibody-drug conjugates were diluted to a 1Ox stock (100 μg/mL) in RPMI+10% FBS. Treatments were then serially diluted 1:10 in RPMI+10% FBS. 20 μL of this series was added to the cells in triplicate, resulting in an 8-point dose curve of antibody-drug conjugate ranging from 10 μg/mL at the highest concentration to 0 vg/mL at the lowest. Plates were incubated at 37° C., 5% CO₂ for 96 hours. At the end of the incubation period, 10 μL of the Substrate Solution was added to each well. The absorbance at 450 nm was measured using a SpectraMax i3× plate reader (Molecular Device, USA). Data were analyzed and graphed using a prism software or Excel software, and the half-maximal inhibitory concentration (IC₅₀) was determined. The results of Steapl-ADC with this experiment are shown in Table 2.

### Example 475. Reduced Molecular Weight and DAR Analysis for the Deglycosylated ADCs by LC-MS

Sample preparation: Reduction of an ADC with 5 mM dithiothreitol at 37° C. for about 2 h, followed by a deglycosylation step with PNGase F at 37° C. overnight generated six or more fragments. HC and LC existed as naked or conjugated forms carrying some payloads. The masses of each ADC fragments and the average DARs of the ADC can be detected. The following equation was used for average DAR calculation for conventional conjugated ADC.

Average DAR=L1/(L0+L1)×2+H1/(H0+H1+H₂+H₃)×2+H₂/(H0+H1+H₂+H₃)×2+H₃/(H0+H1+H₂+H₃)×2.

Method conditions: UPLC system: Waters ACQUITY UPLC H-Class System; Detector: ACQUITY UPLC TUV; Absorption Wavelength: 280 nm; Trap Column: ACQUITY UPLC C4 1.7 μm 2.1×50 mm Column; Mobile phase A: 0.1% formic acid (FA) in water, Mobile phase B: 0.1% formic acid (FA) in ACN; Performed the chromatographic separation at a flow rate of 0.4 ml/min using a linear gradient of mobile phase B (ACN with 0.1% FA) from 5% to 25% for 2 min, followed by 25% to 45% for 8 min, then 45% to 85% for 2 min.

MS conditions: MS system: Waters Xevo-G2XS Q-TOF; Ionization mode: ESI positive; Mass Range: m/z 500-4000 Da. Informatics: the data analysis using UNIFI V1.8.2.169 Software (Waters).

### Example 476. Drug Conjugation Site Analysis for the ADCs by LC-MS

Sample preparation: A recombinant humanized monoclonal antibody-drug conjugate containing a binding ligand of the present invention, Pack size is 100 mg/bottle. ADC samples were denatured and reduced (6M Urea, 10 mM dithiothreitol at 56° C. for about 40 min), alkylated (about 30 mM Iodoacetamide, 40 min in the dark at room temperature), diluted in 50 mM HEPES and digested with trypsin (1/50, enzyme/substrate weight ratio, 4 h, 37° C.).

The drug-loaded peptides of ADCs, the masses of each ADC fragments and the average DARs according to the fragment sequences of the digested ADC were determined by UPLC-MS/MS. MS/MS daughter or product ion spectrum of drug-loaded peptides of the ADC.

Method conditions: LC system: Waters ACQUITY UPLC H-Class System; Detector: ACQUITY UPLC TUV, Absorption Wavelength: 214 nm; Trap Column: ACQUITY UPLC C18 1.7 μm 2.1×100 mm Column; Mobile phase A: 0.1% formic acid (FA) in water, Mobile phase B: 0.1% formic acid (FA) in ACN; Perform the chromatographic separation at a flow rate of 0.2 μl/min using a linear gradient of mobile phase B (ACN with 0.1% FA) from 1% to 40% over 95 min., followed by 40% to 80% for 15 min.;

MS conditions: MS system: Waters Xevo-G2XS Q-TOF; Ionization mode: ESI positive, Sensitivity Mode; Data Acquisition: MS^{E}; Mass Range: m/z 100-2500 Da; Informatics: Perform the data analysis using UNIFI V1.8.2.169 Software (Waters).

### Example 477. DAR analysis

DAR was analyzed by using HIC-HPLC, and the HPLC parameters are as follow Table 1:

TABLE 1
The condition for DAR analysis by HIC-HPLC.

| HPLC | Agilent 1260 | | | | | |
|---|---|---|---|---|---|---|
| Column | Thermo HIC butyl 4.6 × 100 mm | | | | | |
| Phase A | 0.5M (NH₄)₂SO₄ + 100 mM NaH₂PO₄, | | | | | |
| Phase B | 100 mM NaH₂PO₄, | | | | | |
| Sample | Dilute with buffer A to about 2 mg/mL, | | | | | |
| | injection volume 10 μL | | | | | |
| Rate | 0.8 mL/min | | | | | |
| Wavelength | 280 nm | | | | | |
| Column Temp. | 30° C. | | | | | |
| | Time (min) | 0 | 35 | 40 | 41 | 45 |
| Gradient | Phase A (%) | 80 | 0 | 0 | 80 | 80 |
| | Phase B (%) | 20 | 100 | 100 | 20 | 20 |

### Example 478. The structures of the conjugates that were prepared by both the traditional conjugation process and the homogeneous conjugation process are illustrated below: wherein mAb is an antibody, n -1~20, preferably n -2~8

mAbs used for the conjugation have the following information: The Steap1, Trop2, Folate, Her2, and EGFR antibodies were referred from the same of CDRs of vandortuzumab, sacituzumab, farletuzumab, trastuzumab, and nimotuzumab respectively. DLL3, PSMA, cMet, Muc1, Guyc2C (GCC), Her2 bispecific (trastuzumab/pertuzumab), Her3 and B7H3 antibodies were generated in house through hybridoma technique and then humanization, and their sequences and production will be filed for patent application later accordingly. For DAR <5, the homogeneous conjugation process described above was used for preparation of ADCs of this patent. For DAR >6, the traditional thiol-maleimide conjugation was applied for this application. The structures of the generated ADCs in this patent application are illustrated below:

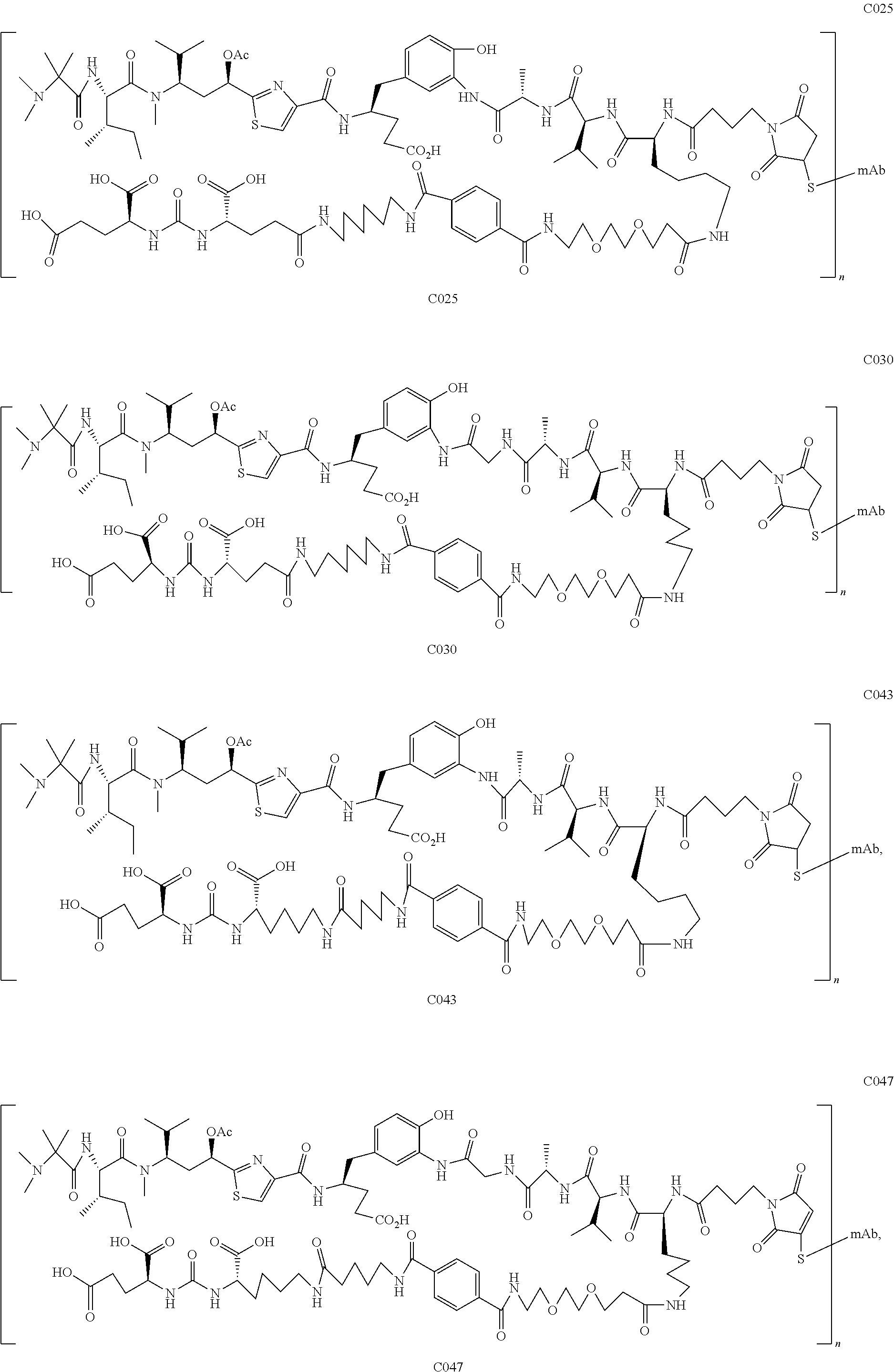

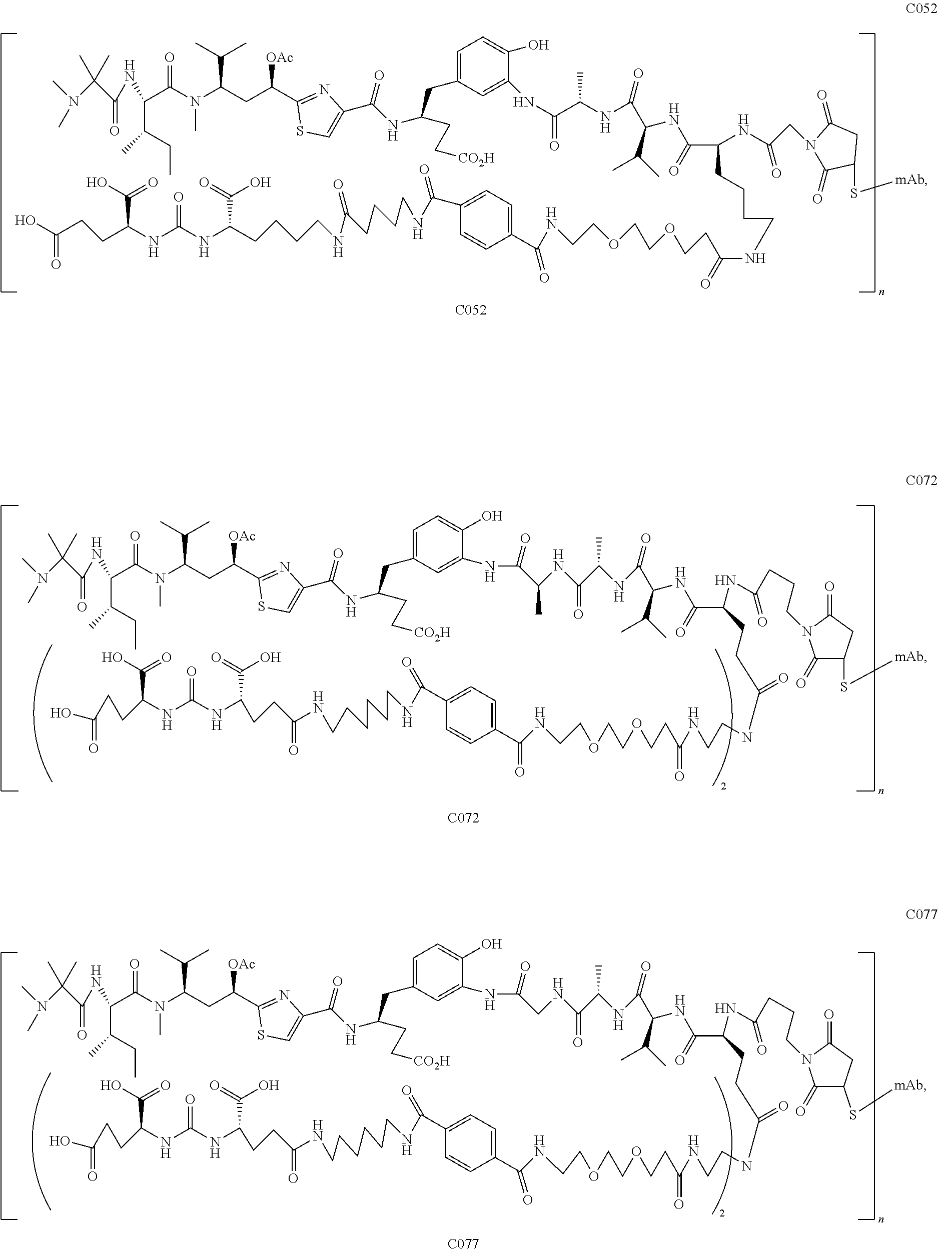

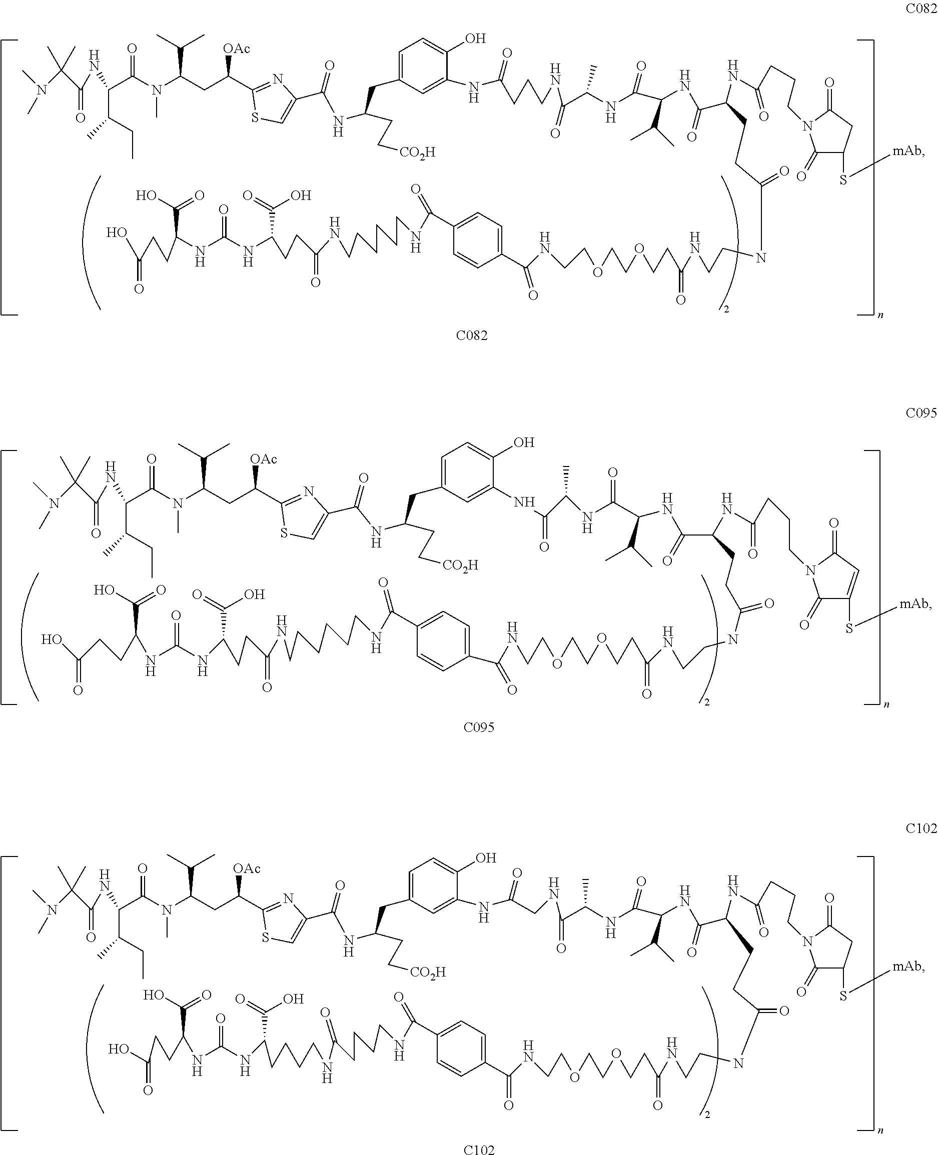

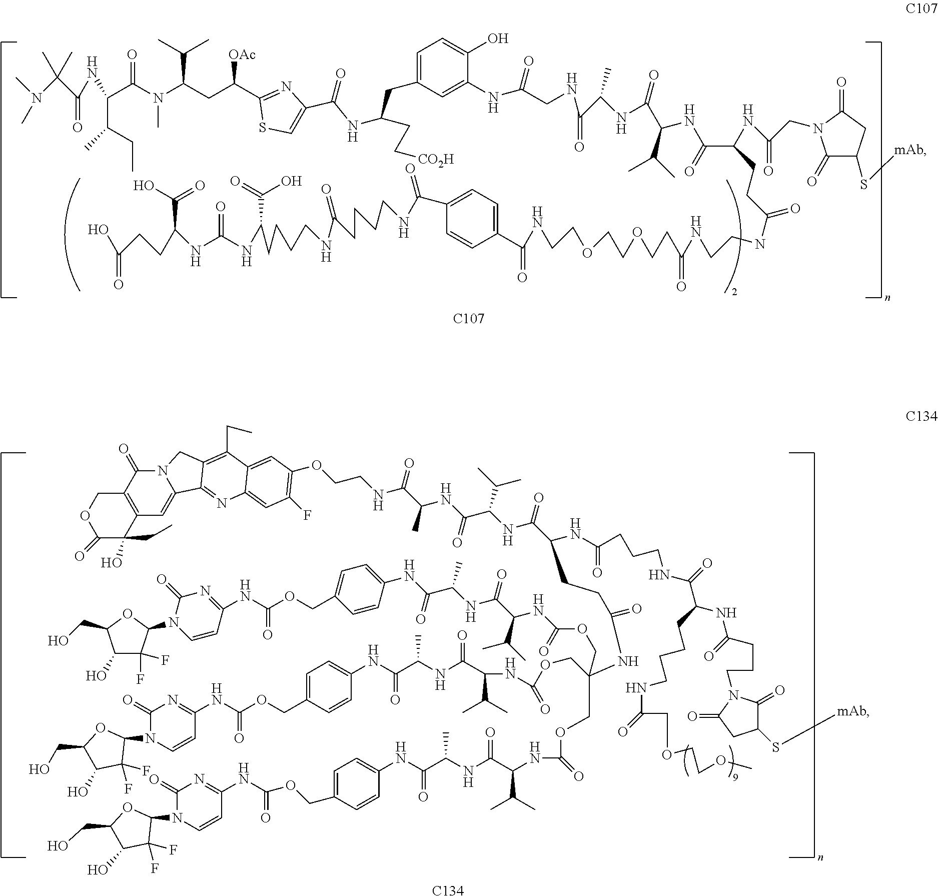

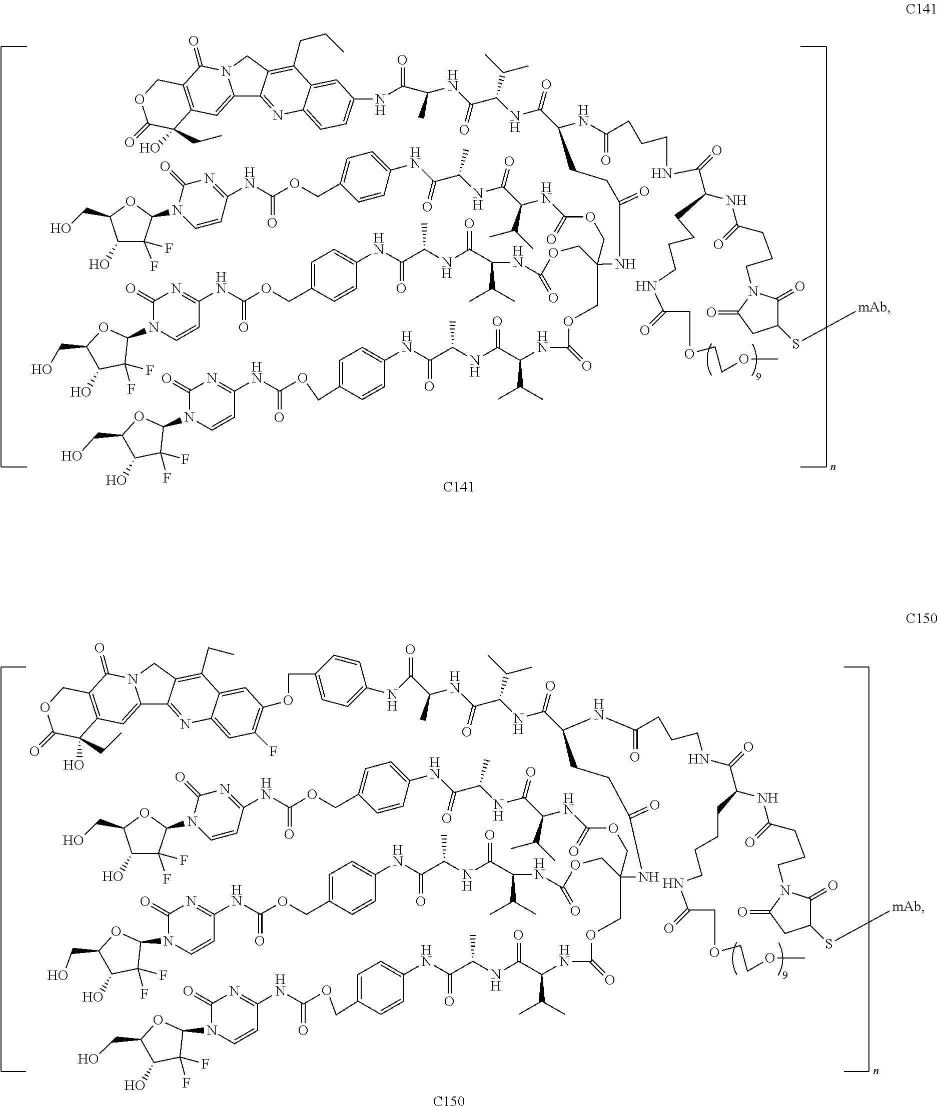

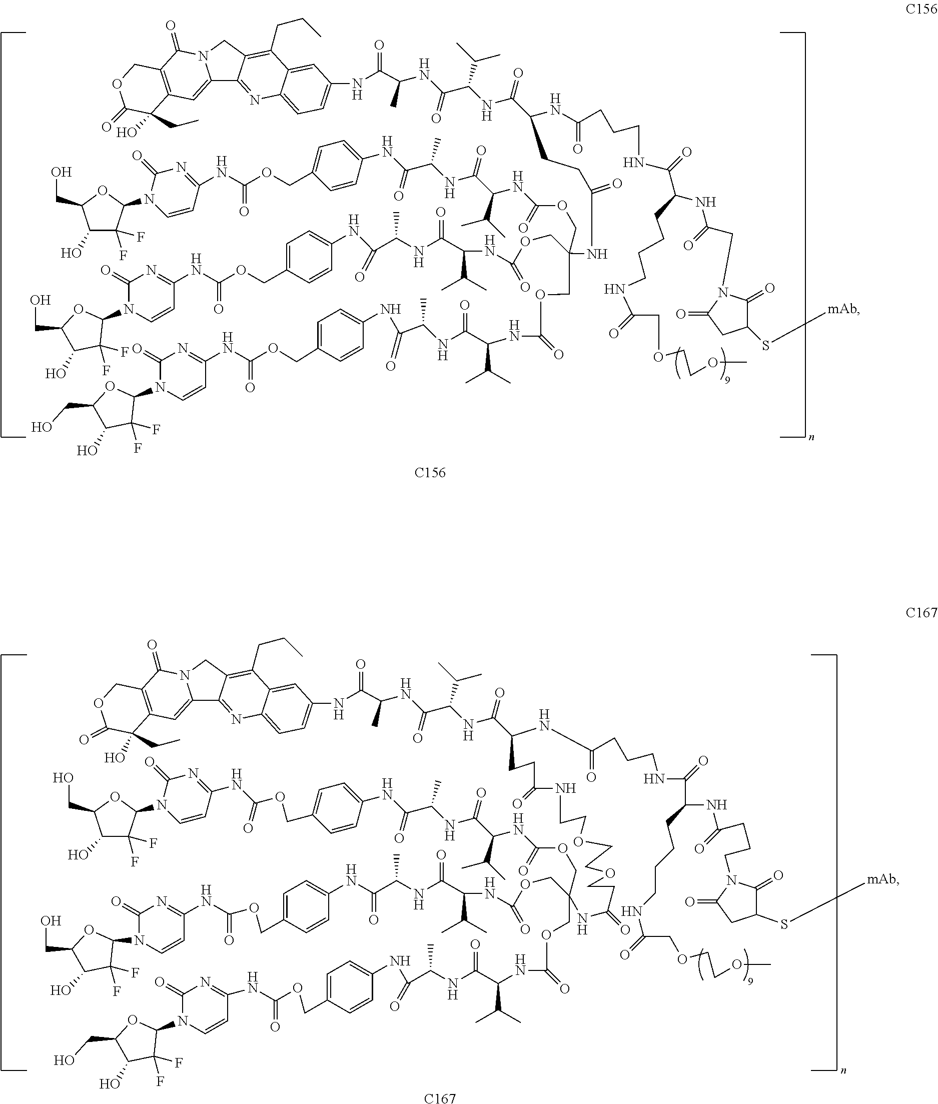

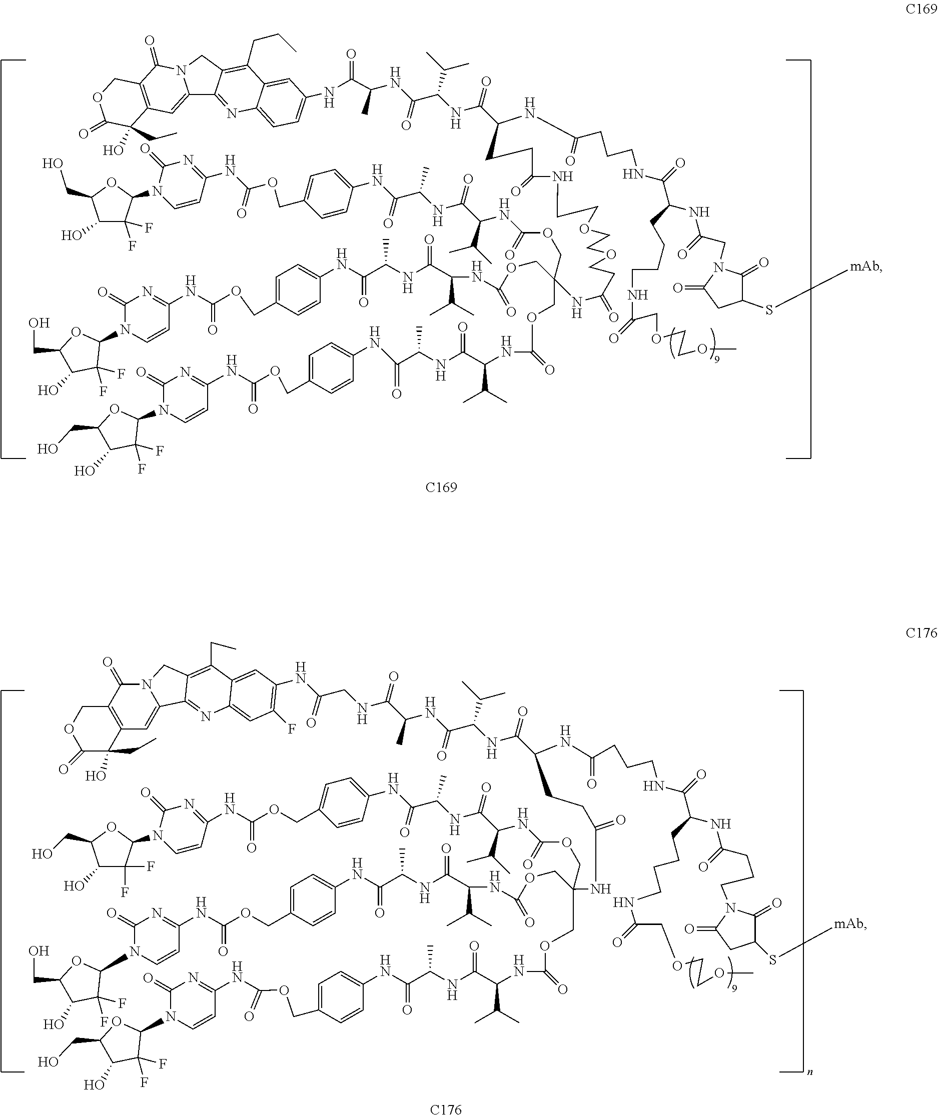

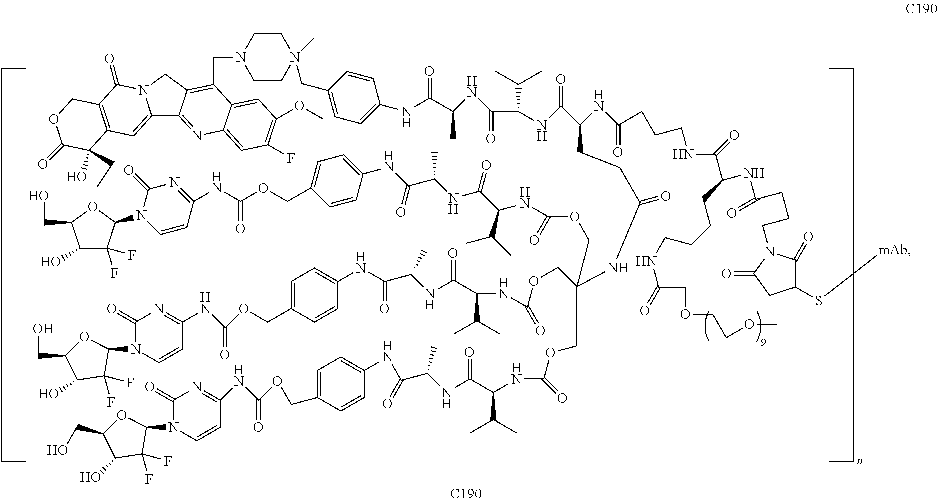

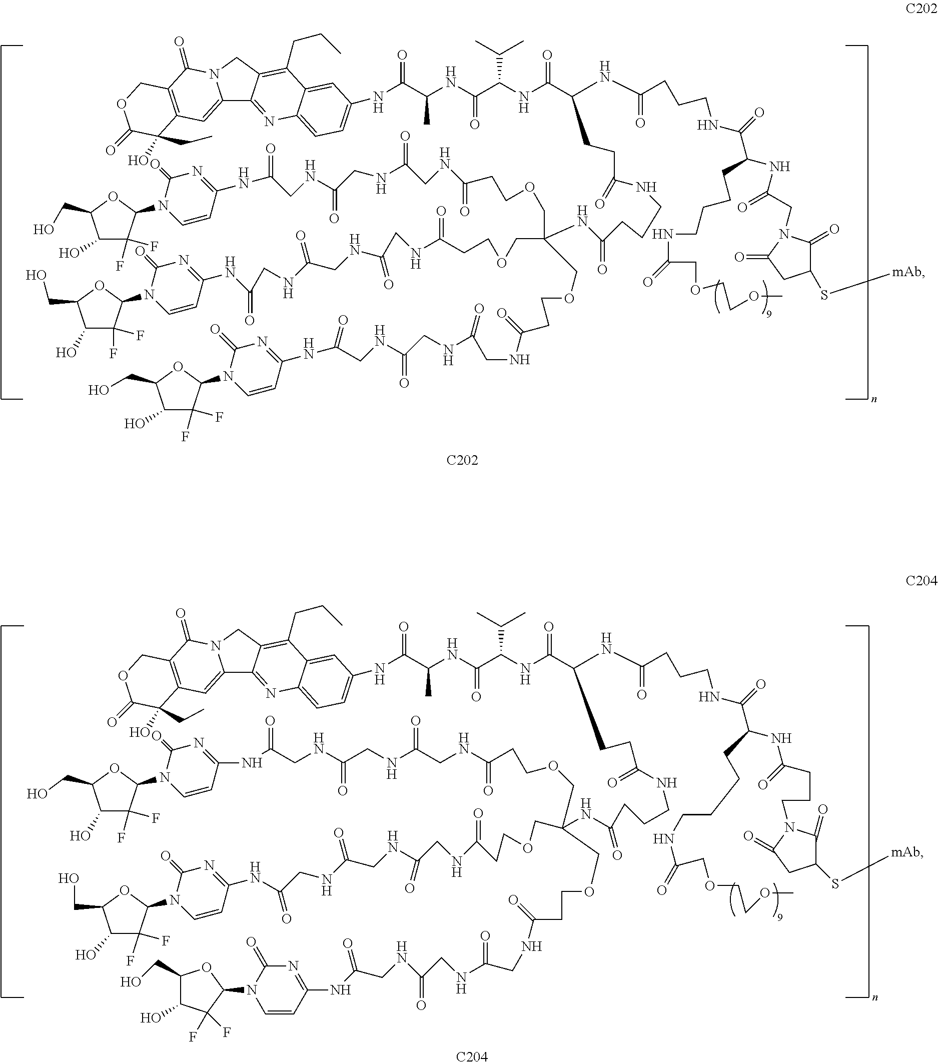

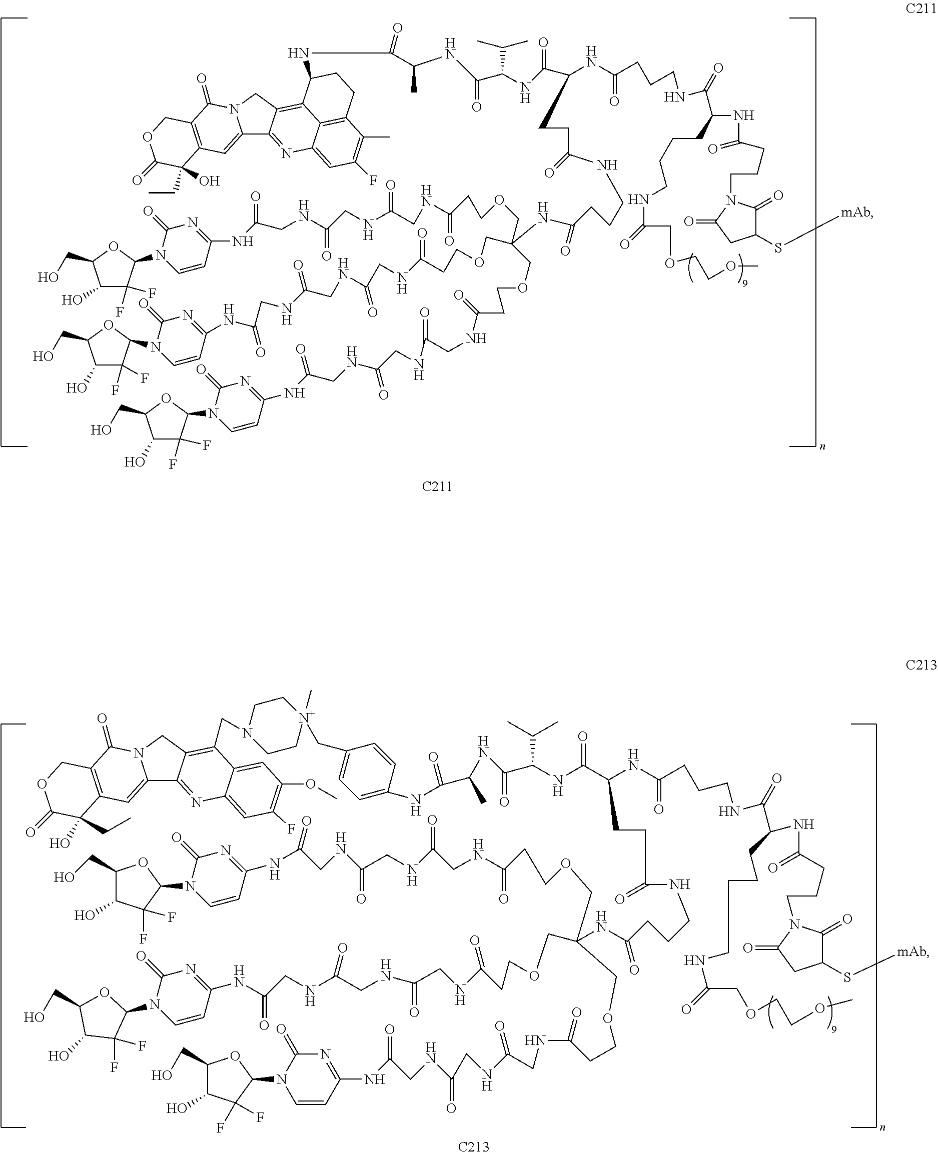

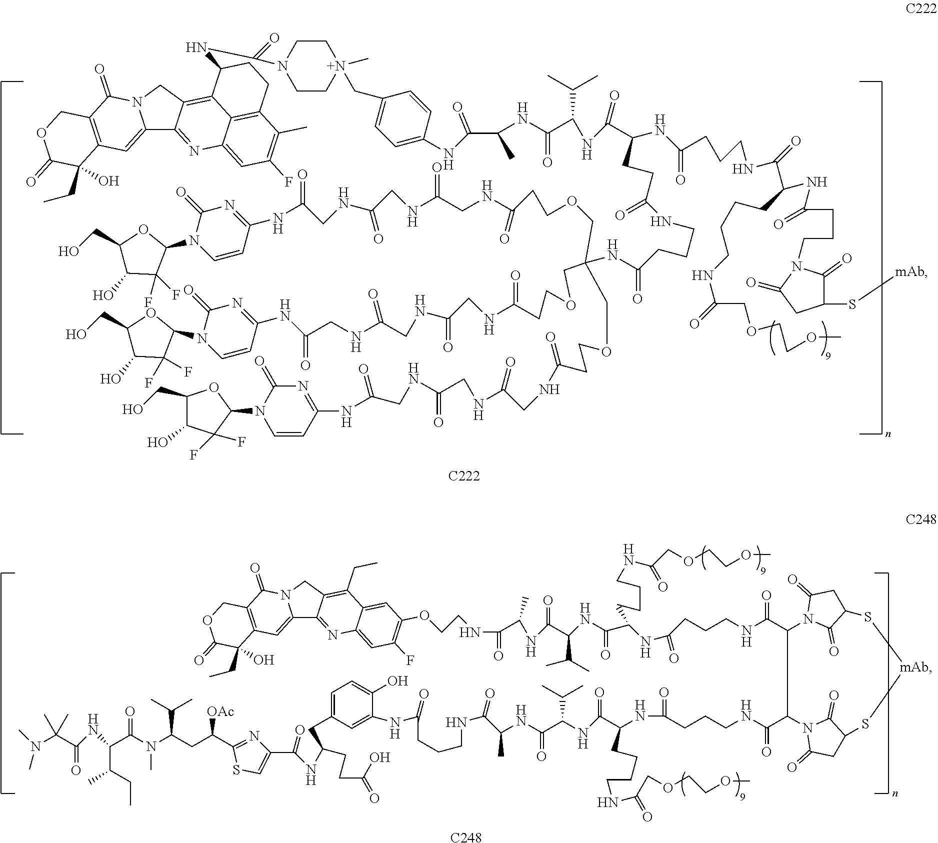

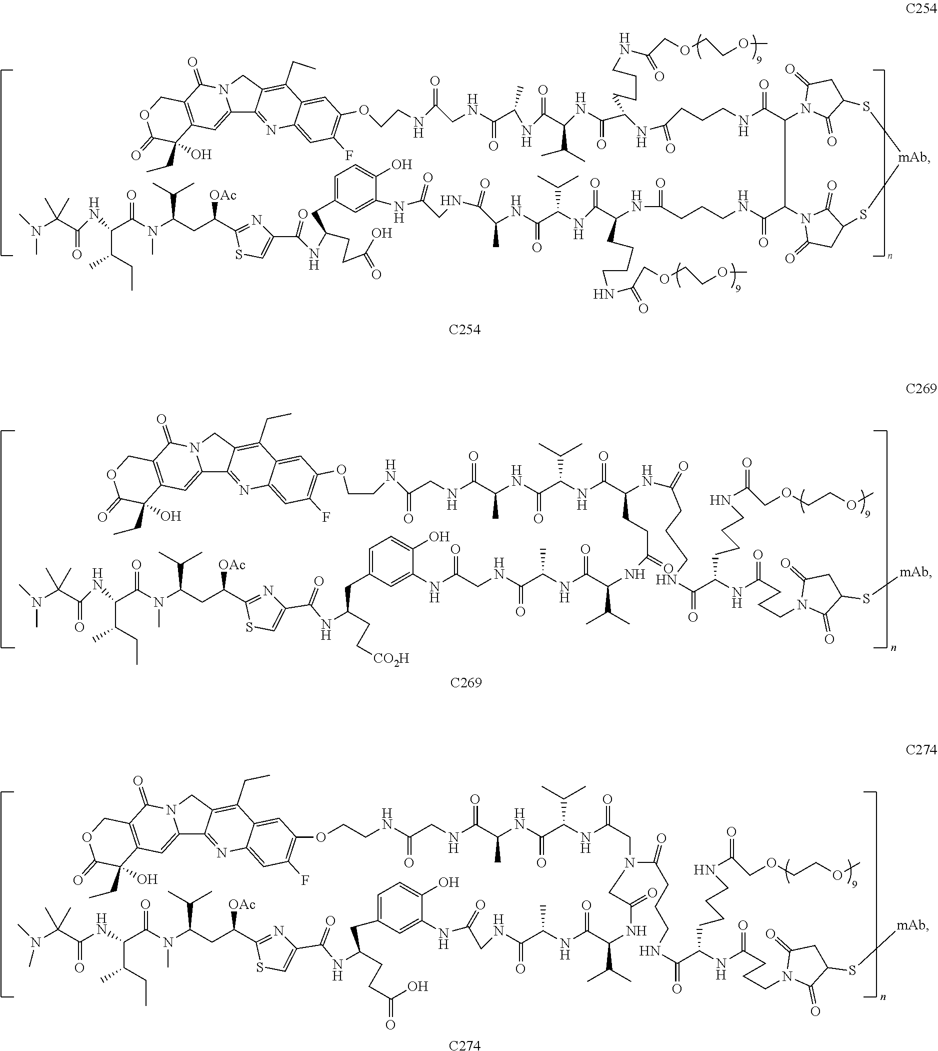

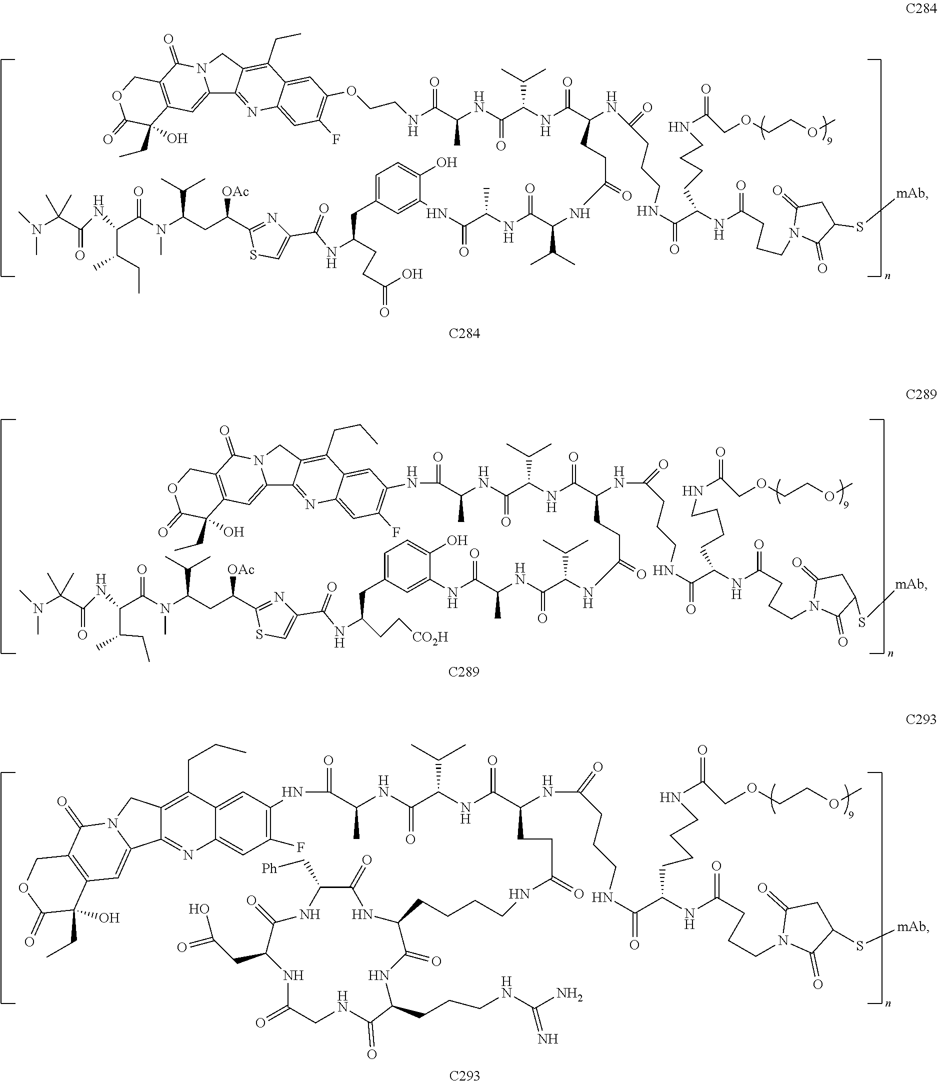

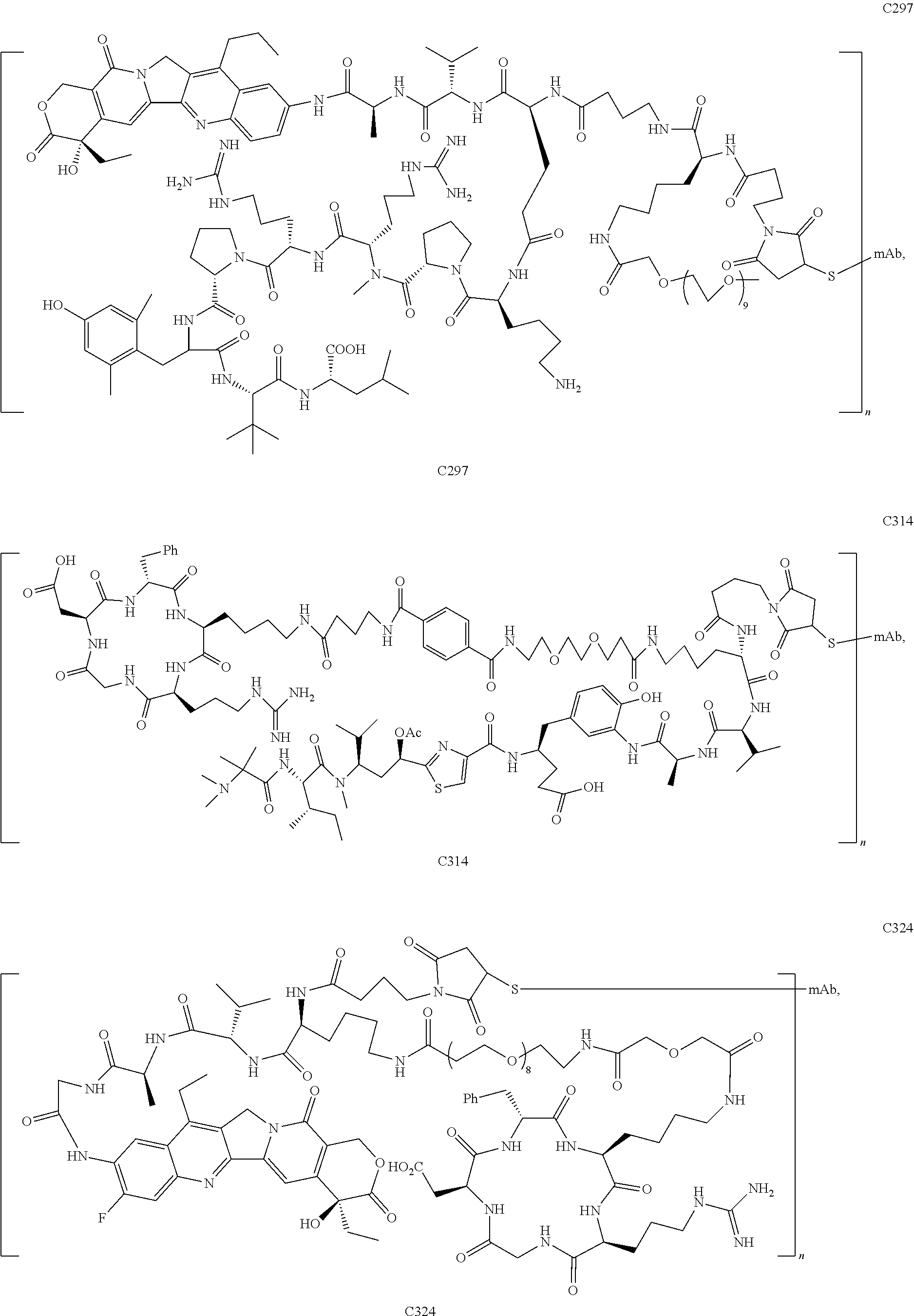

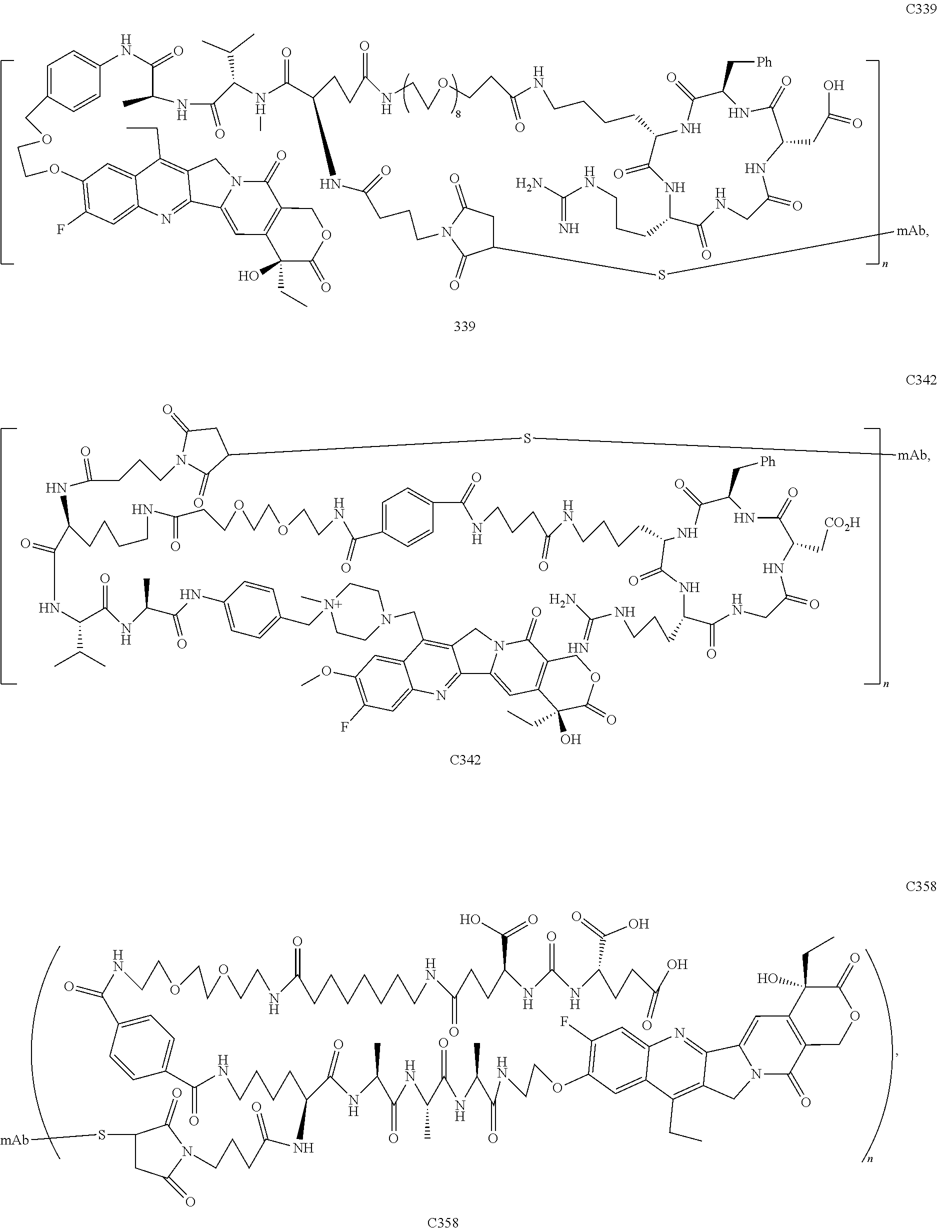

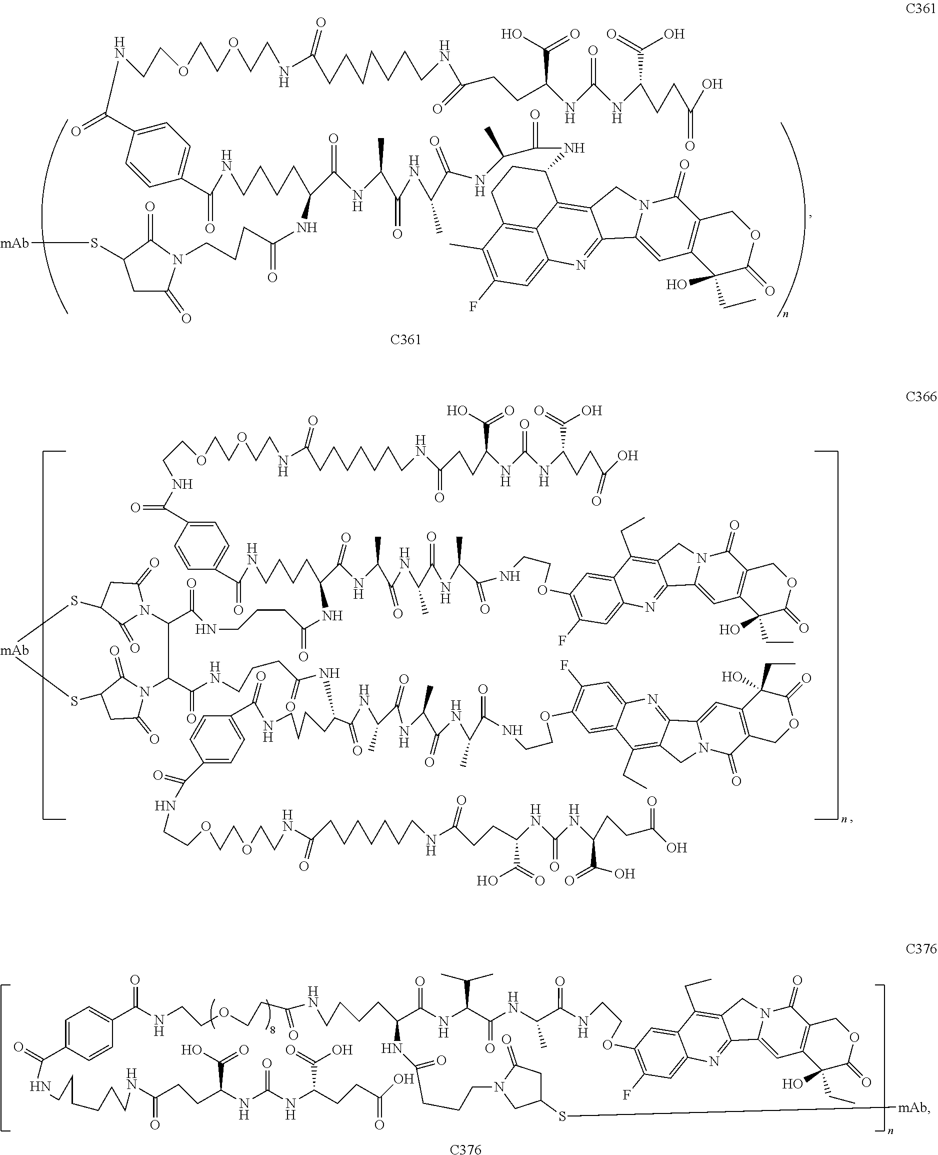

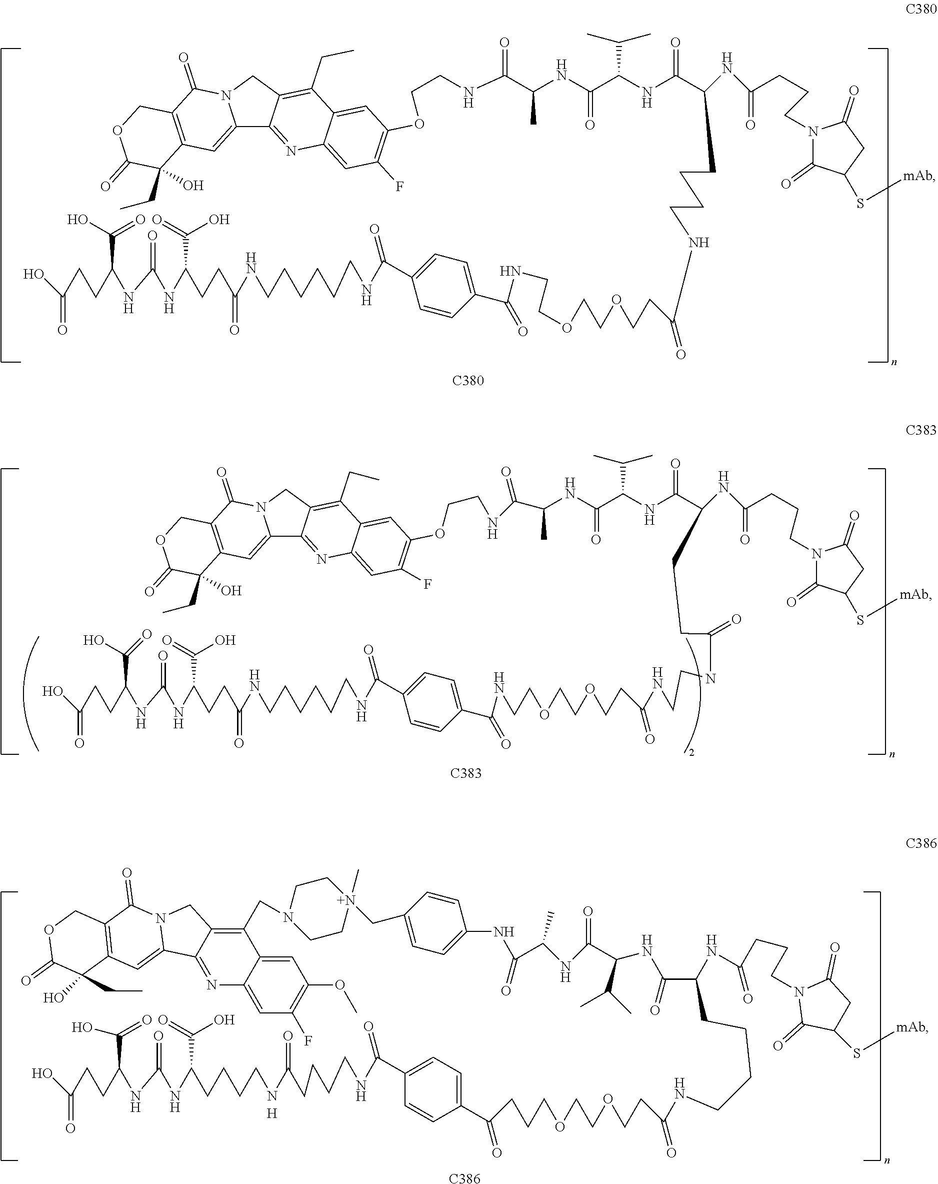

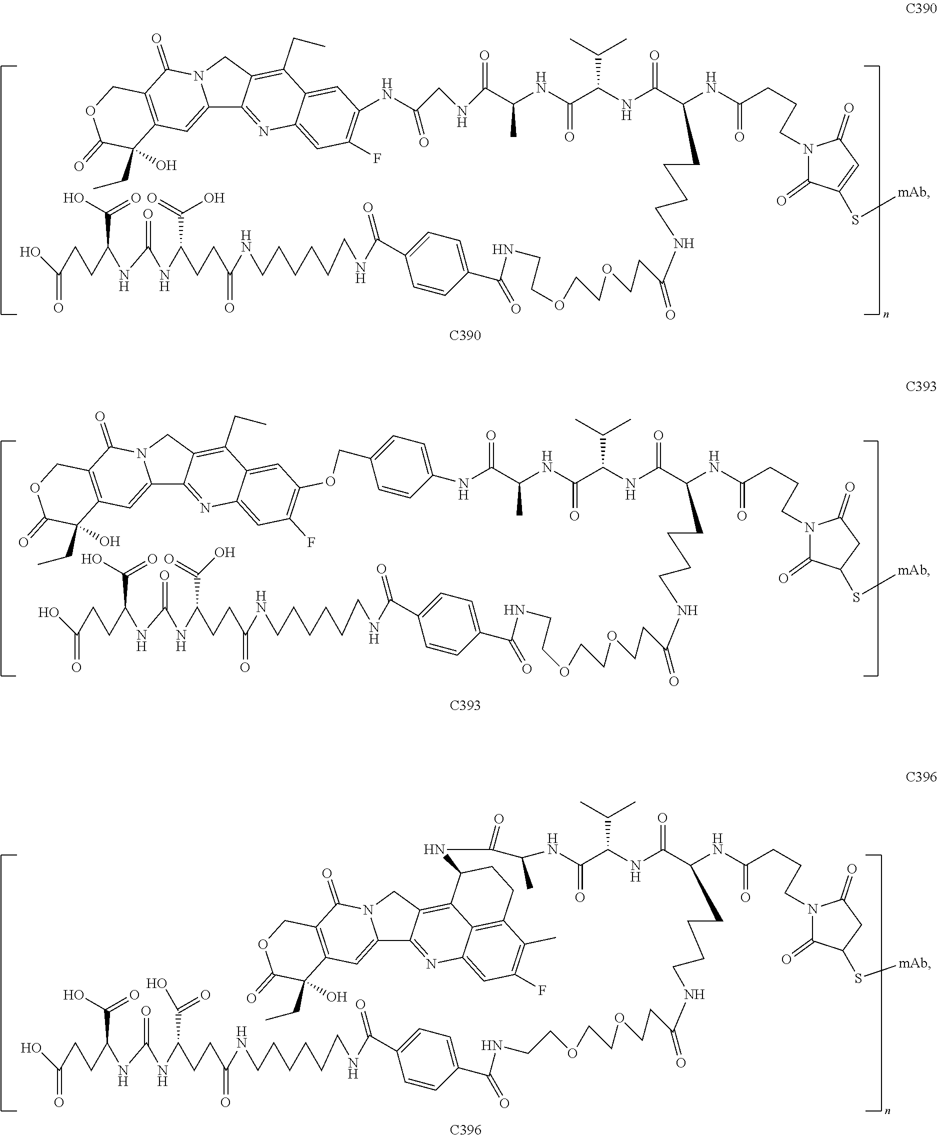

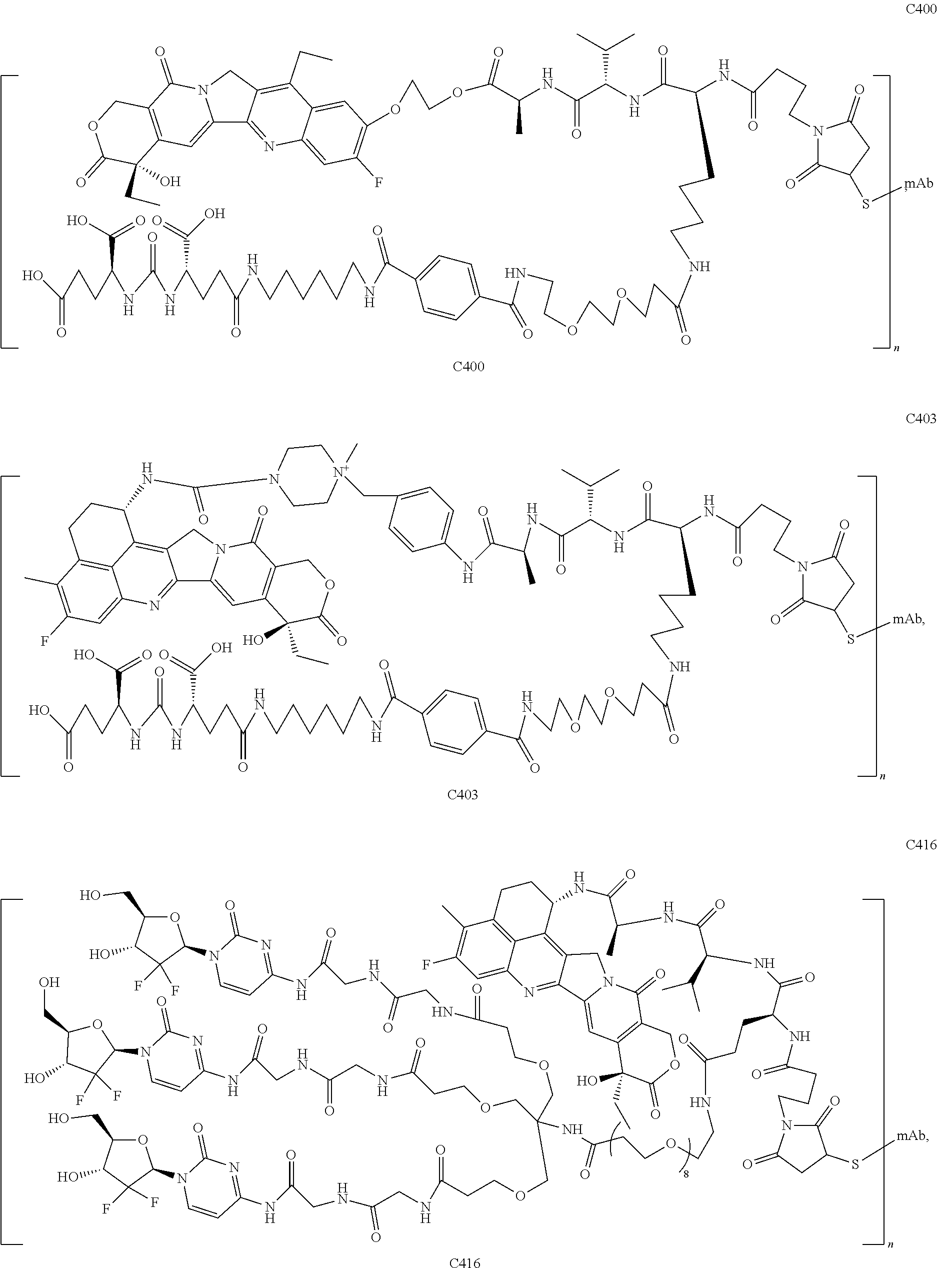

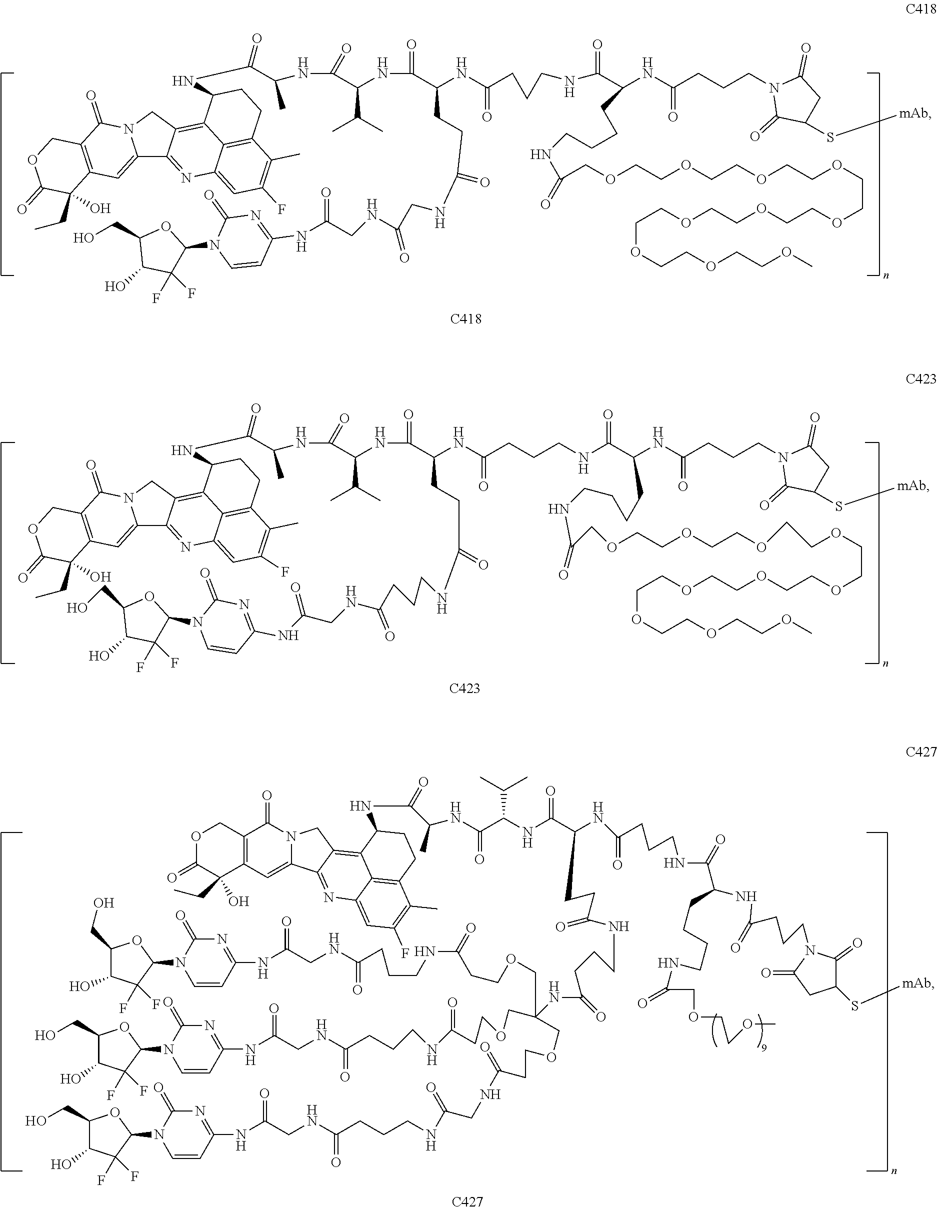

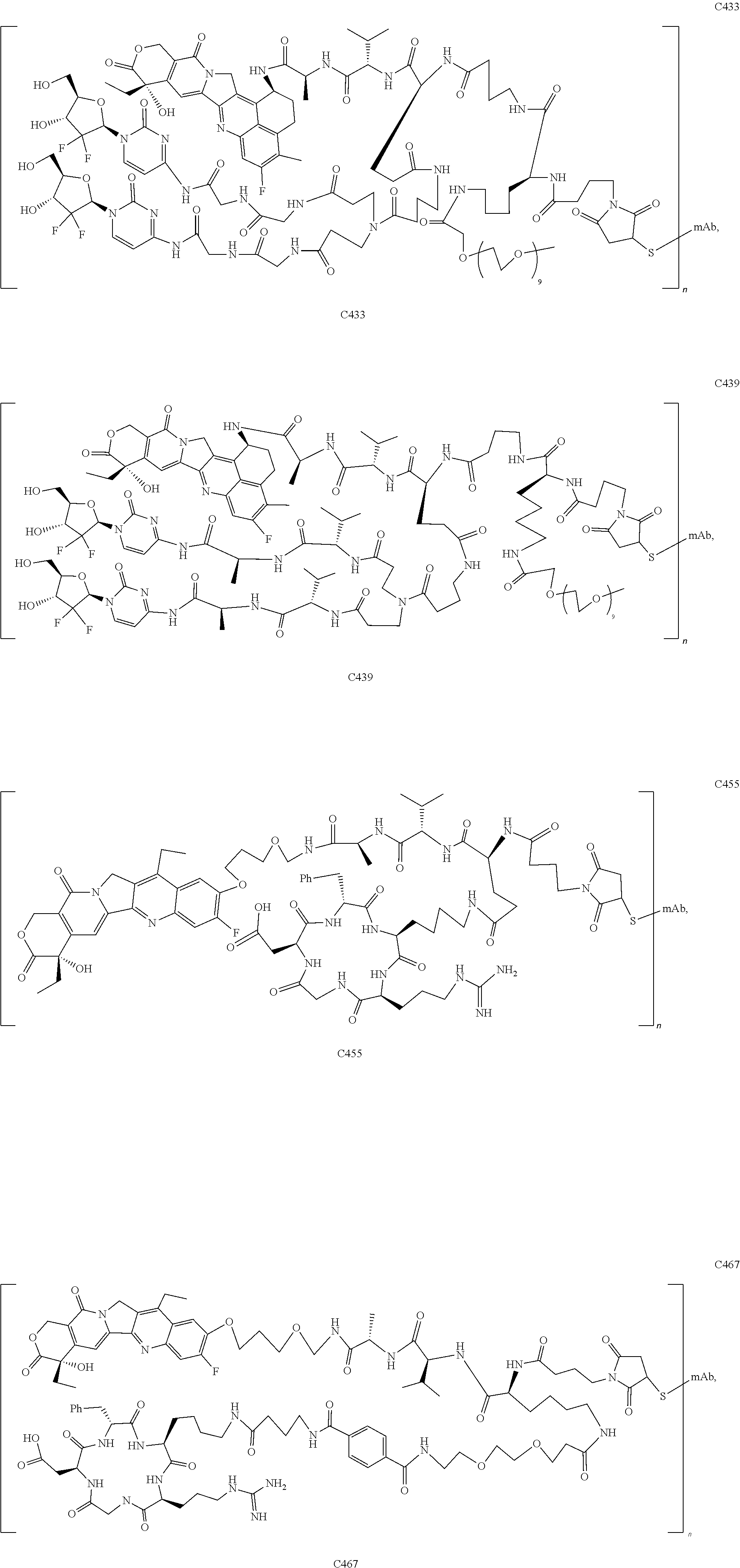

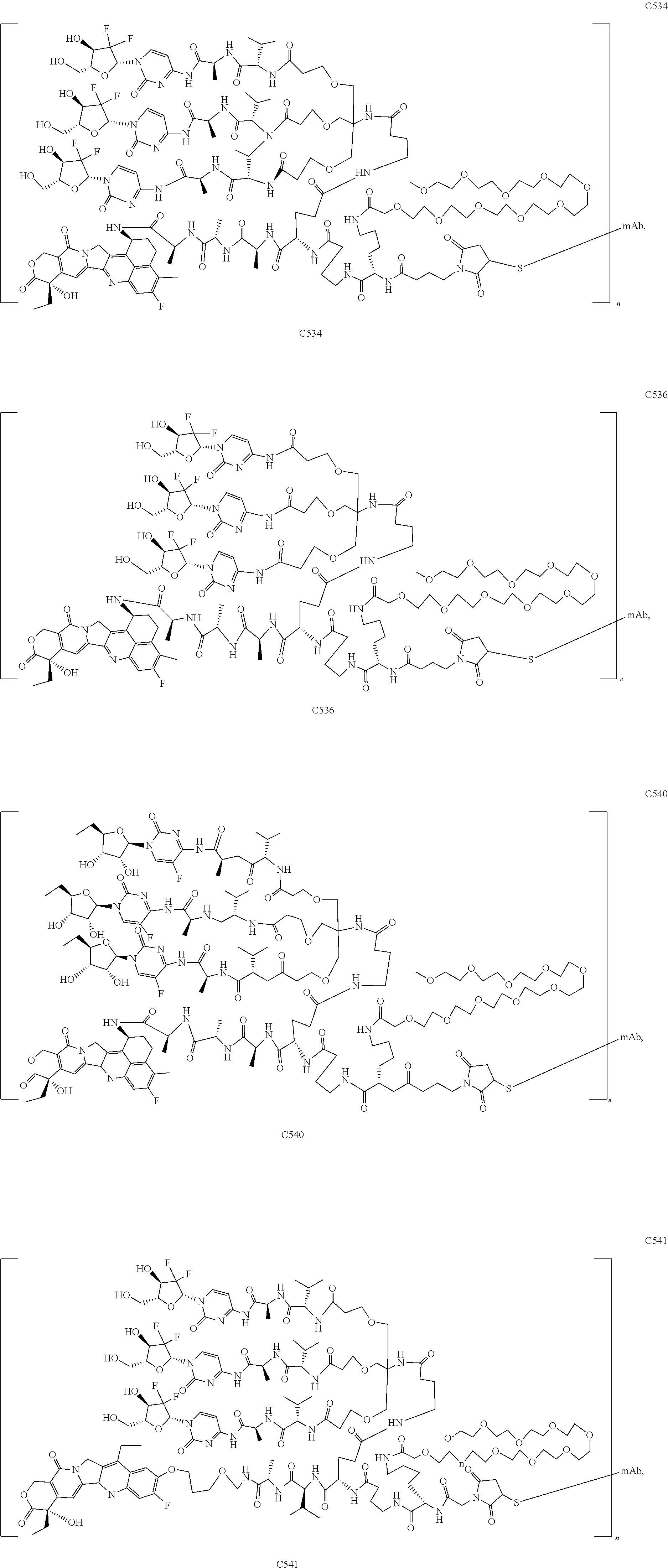

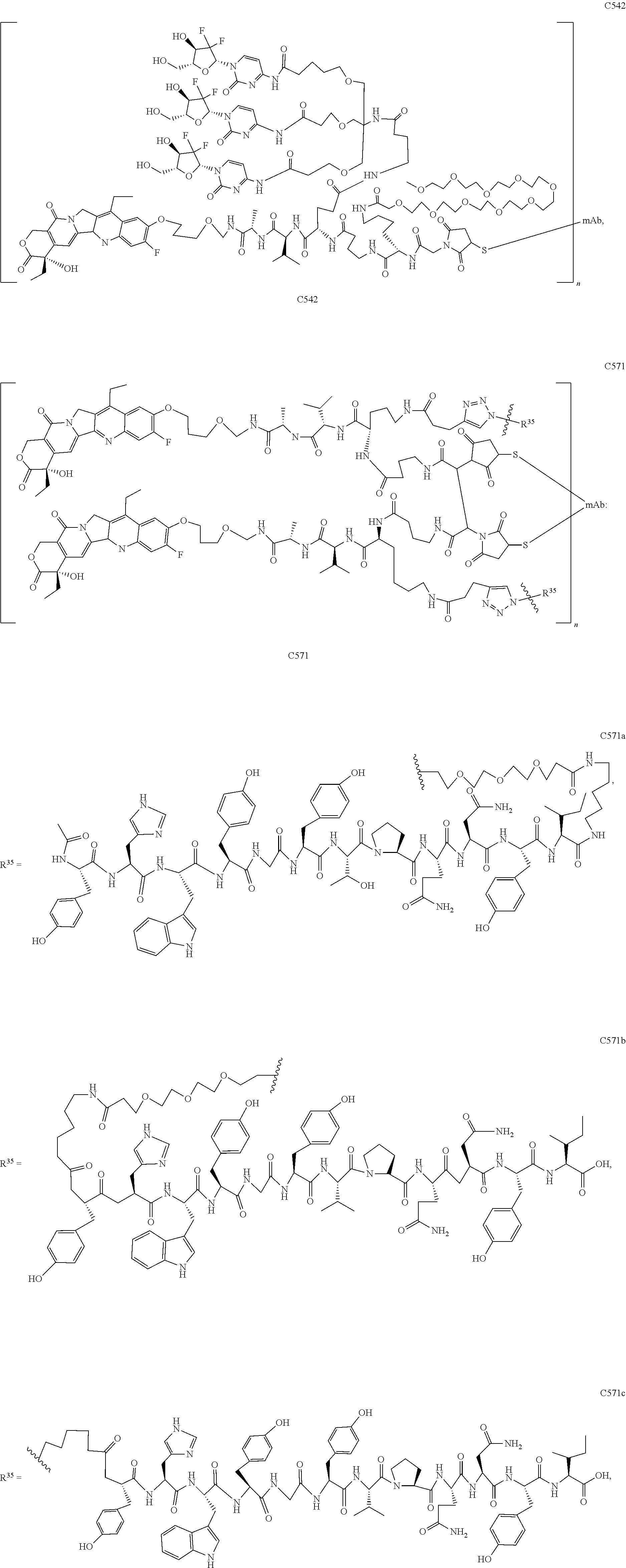

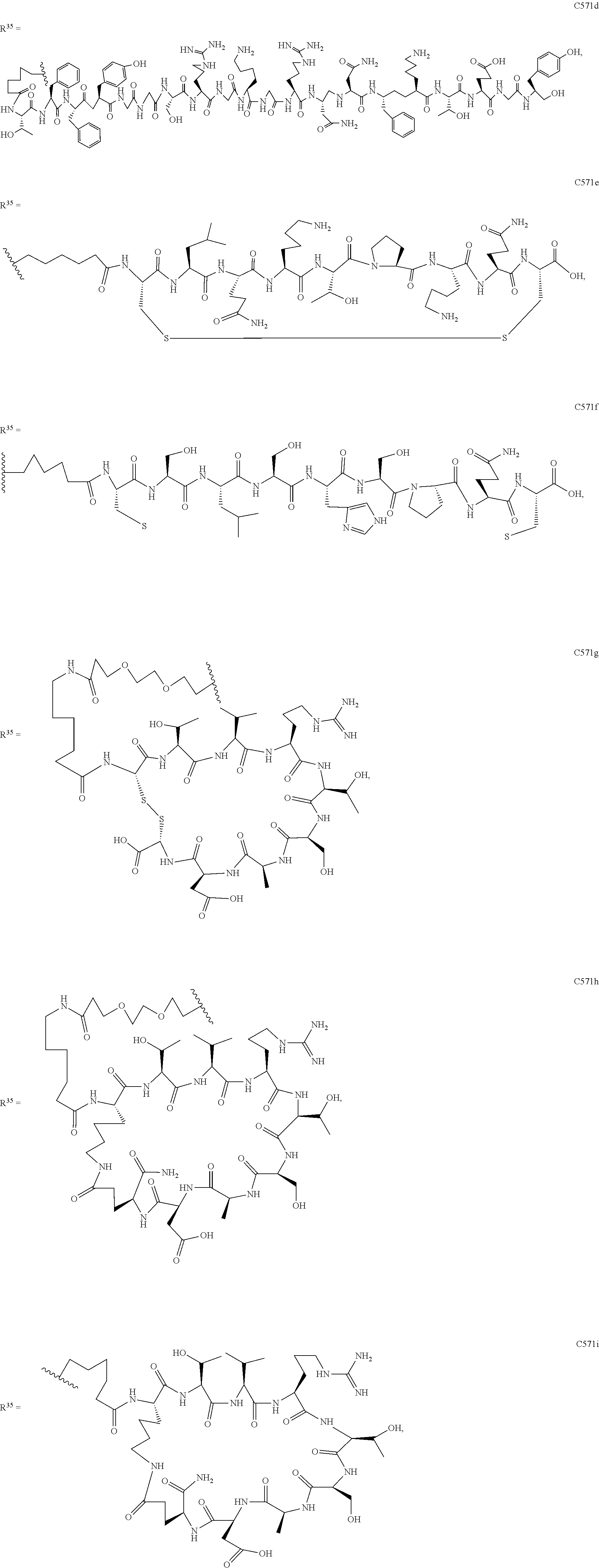

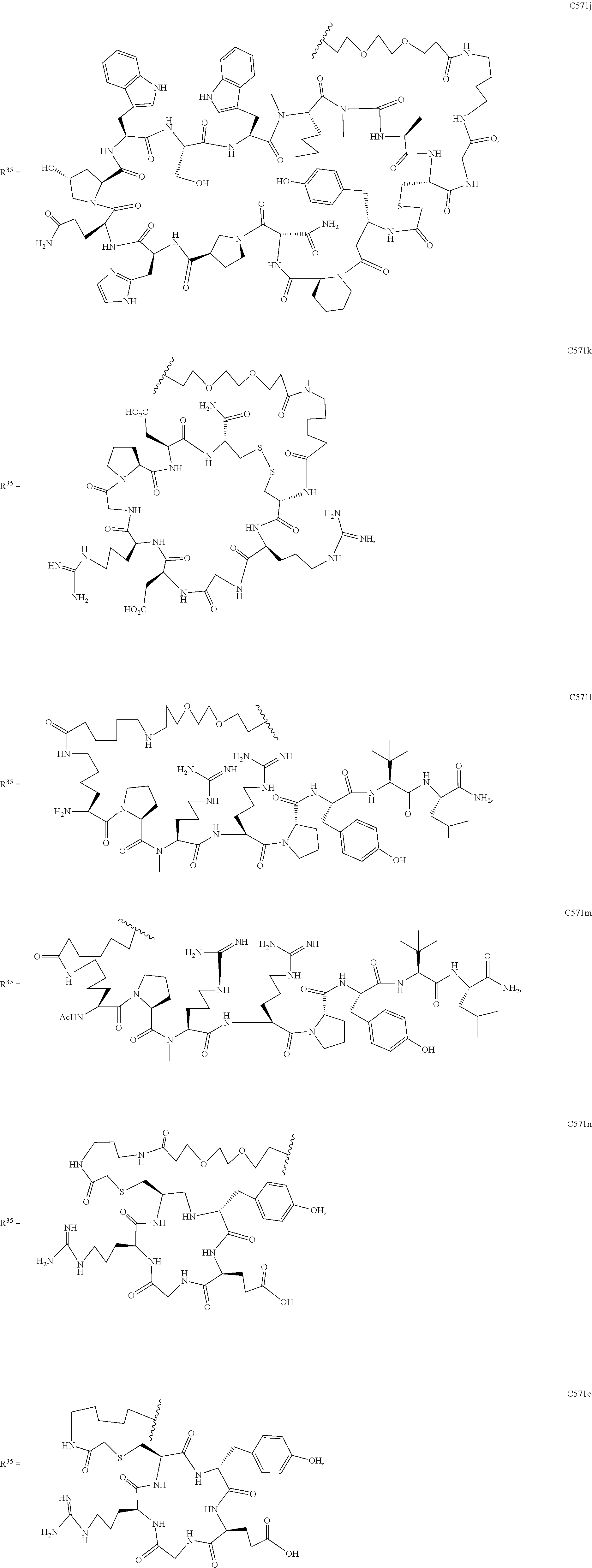

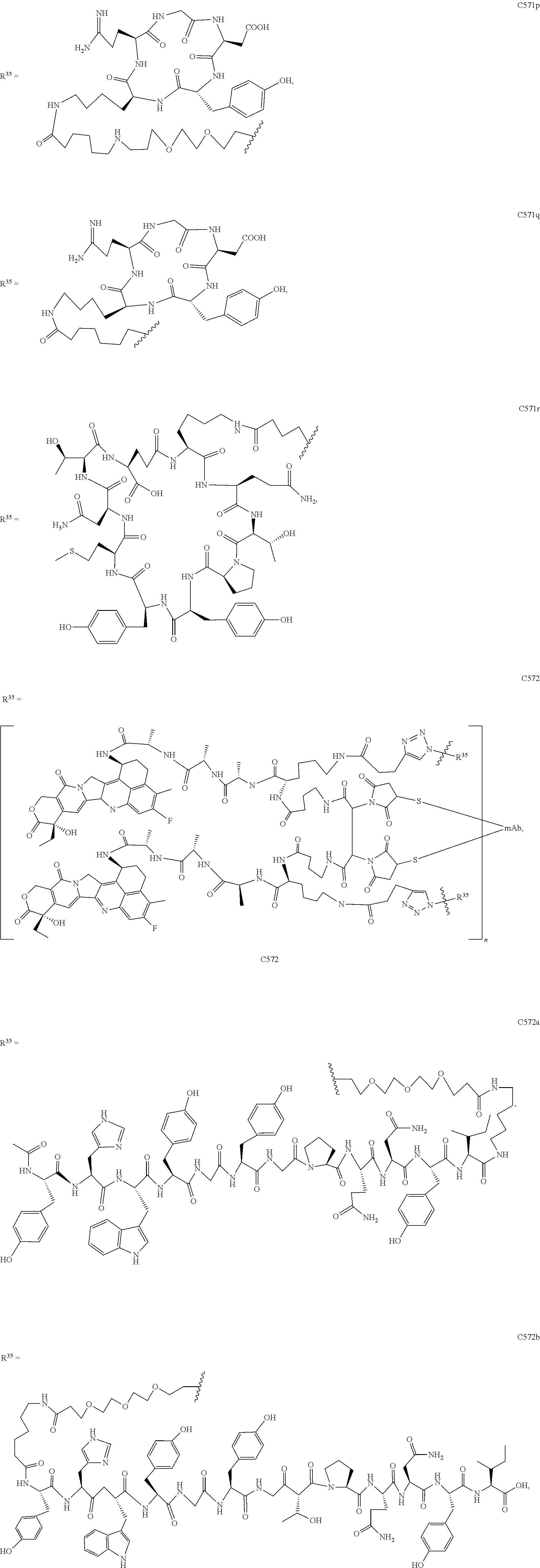

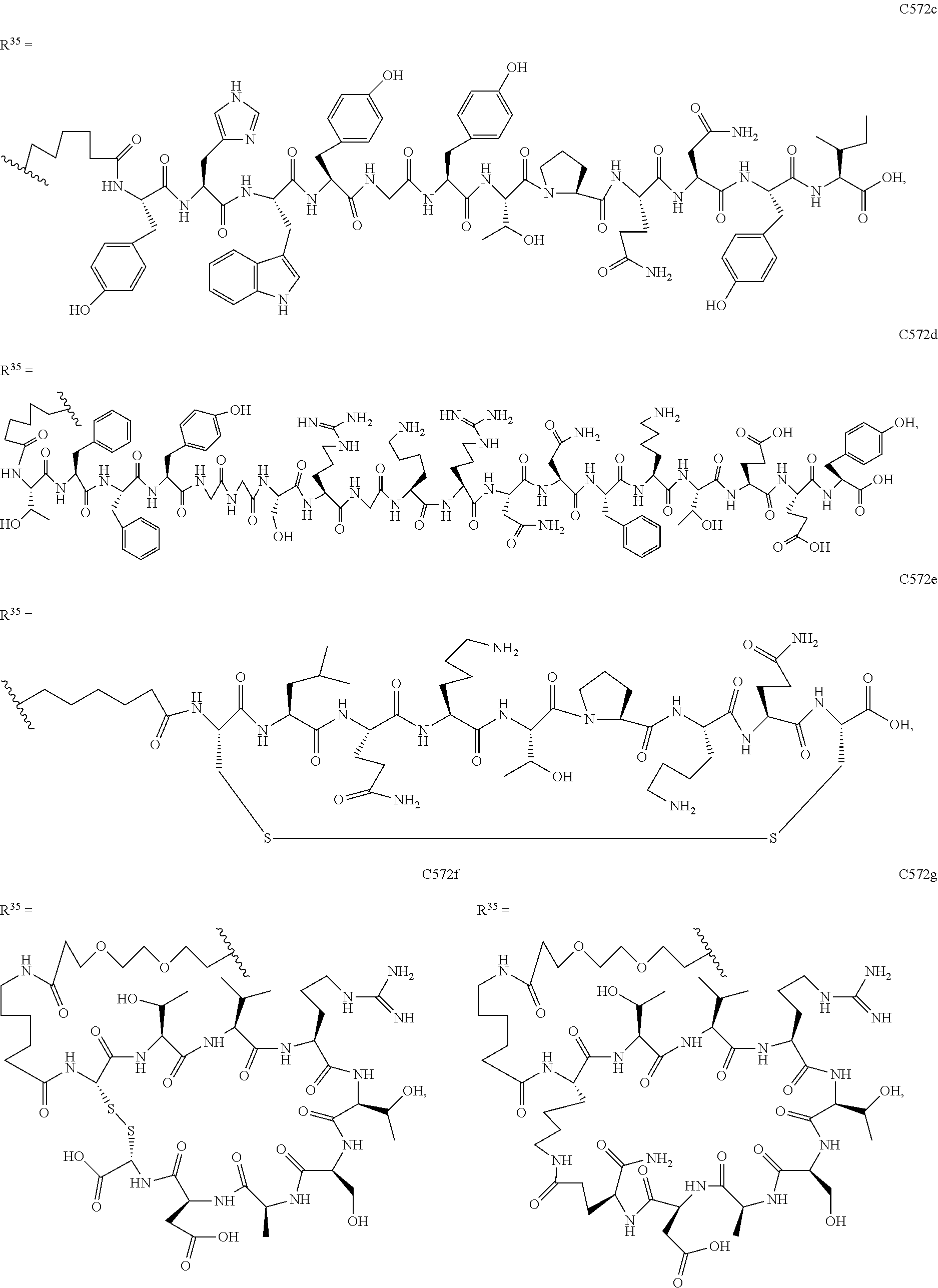

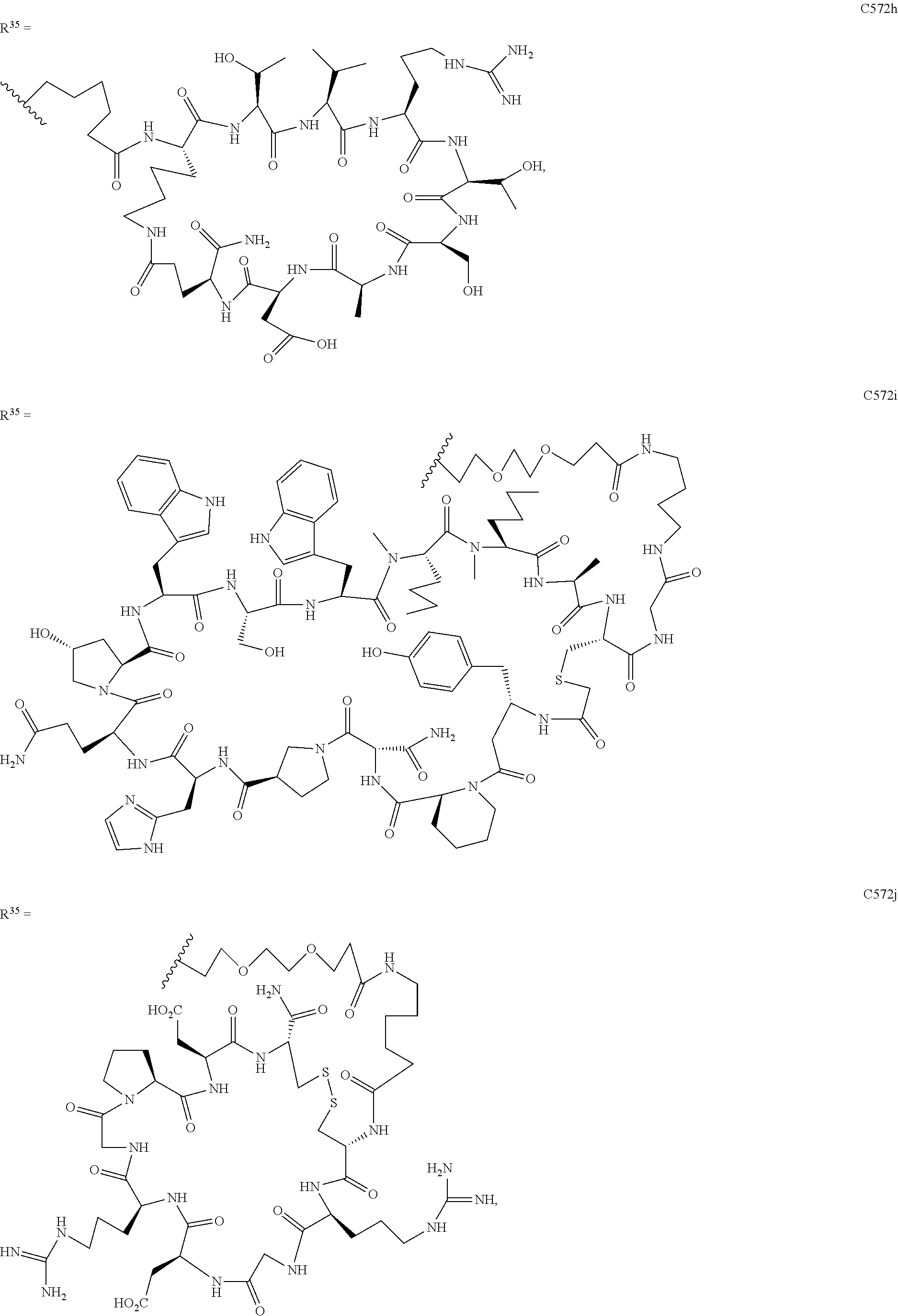

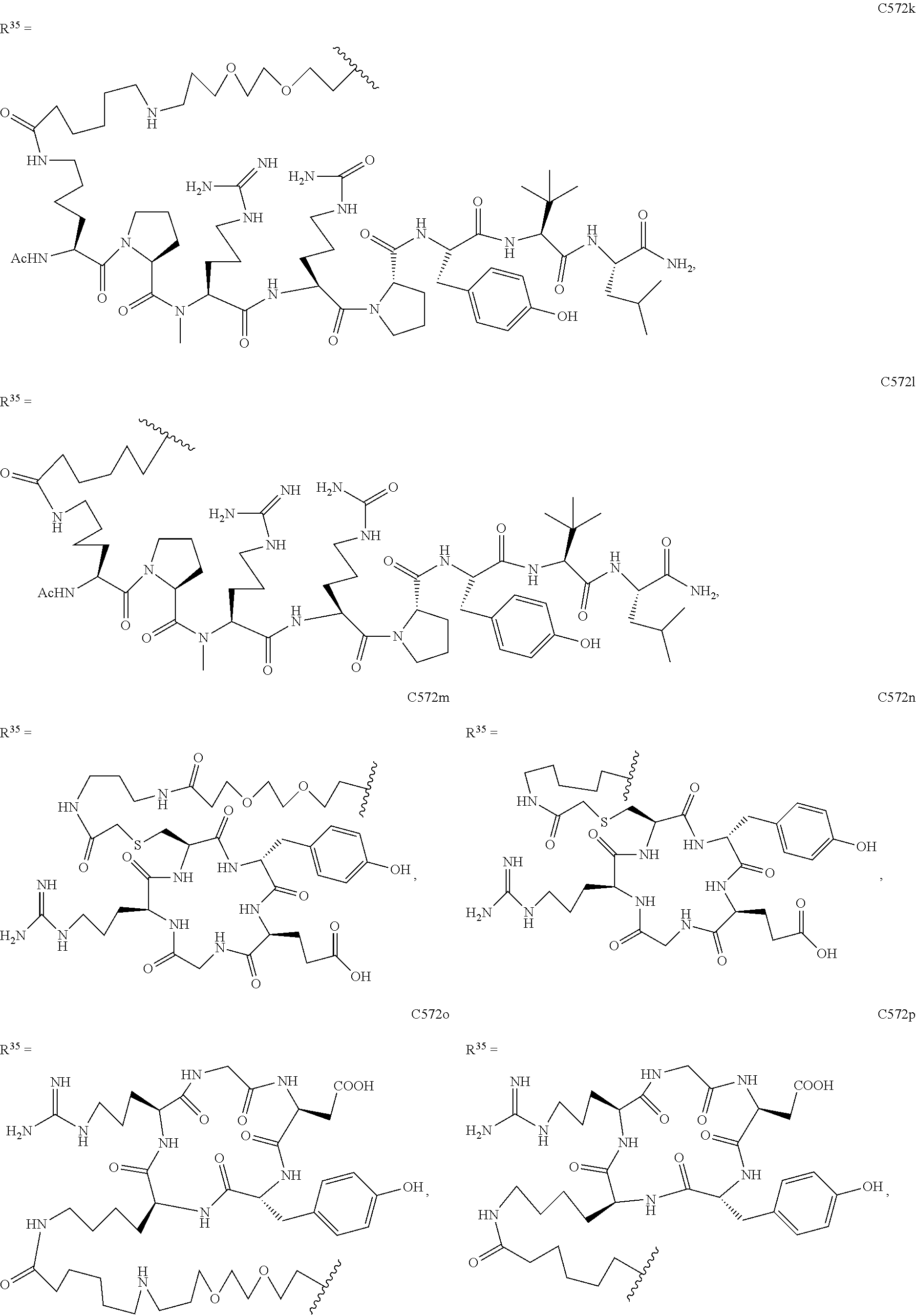

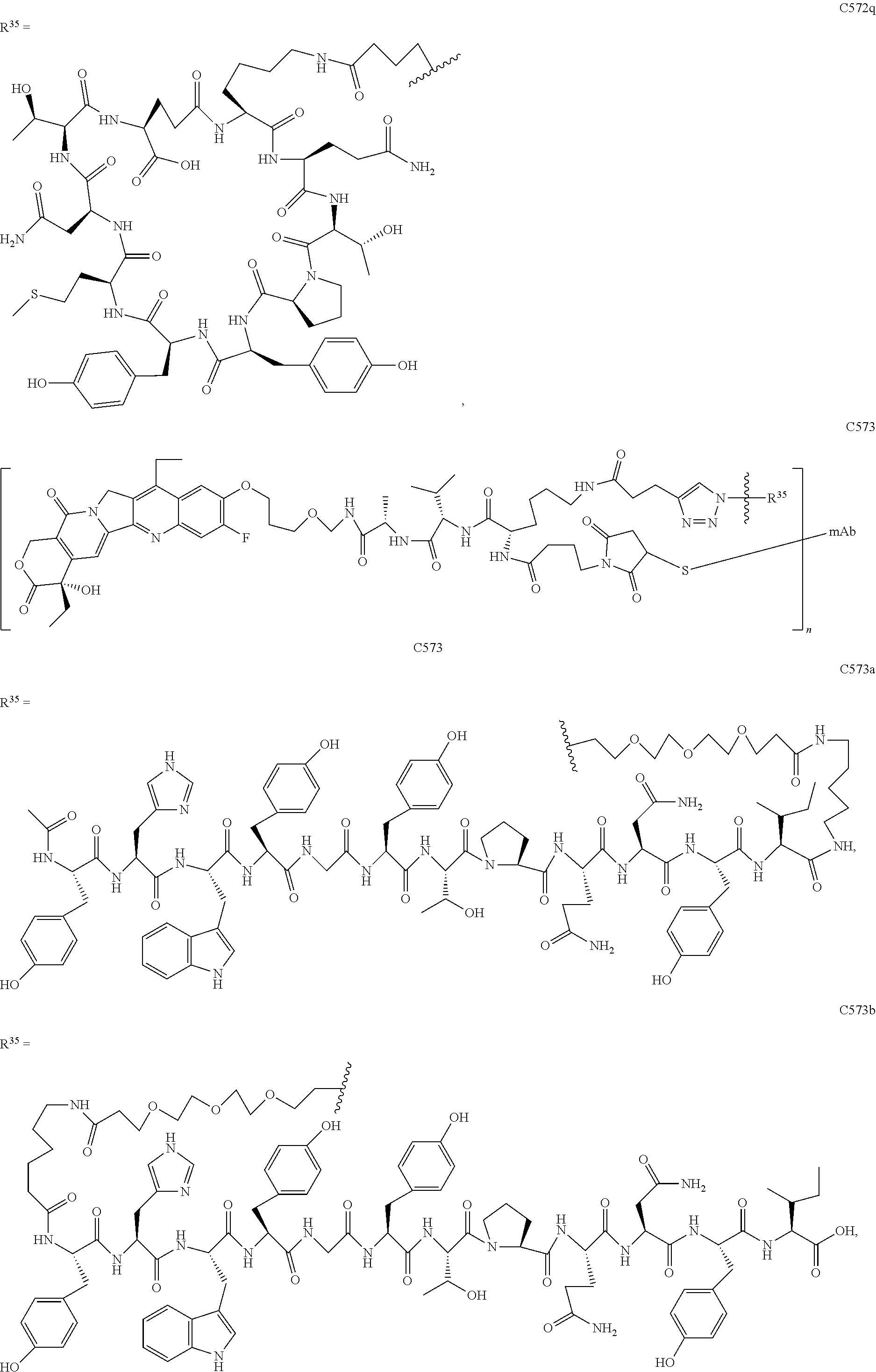

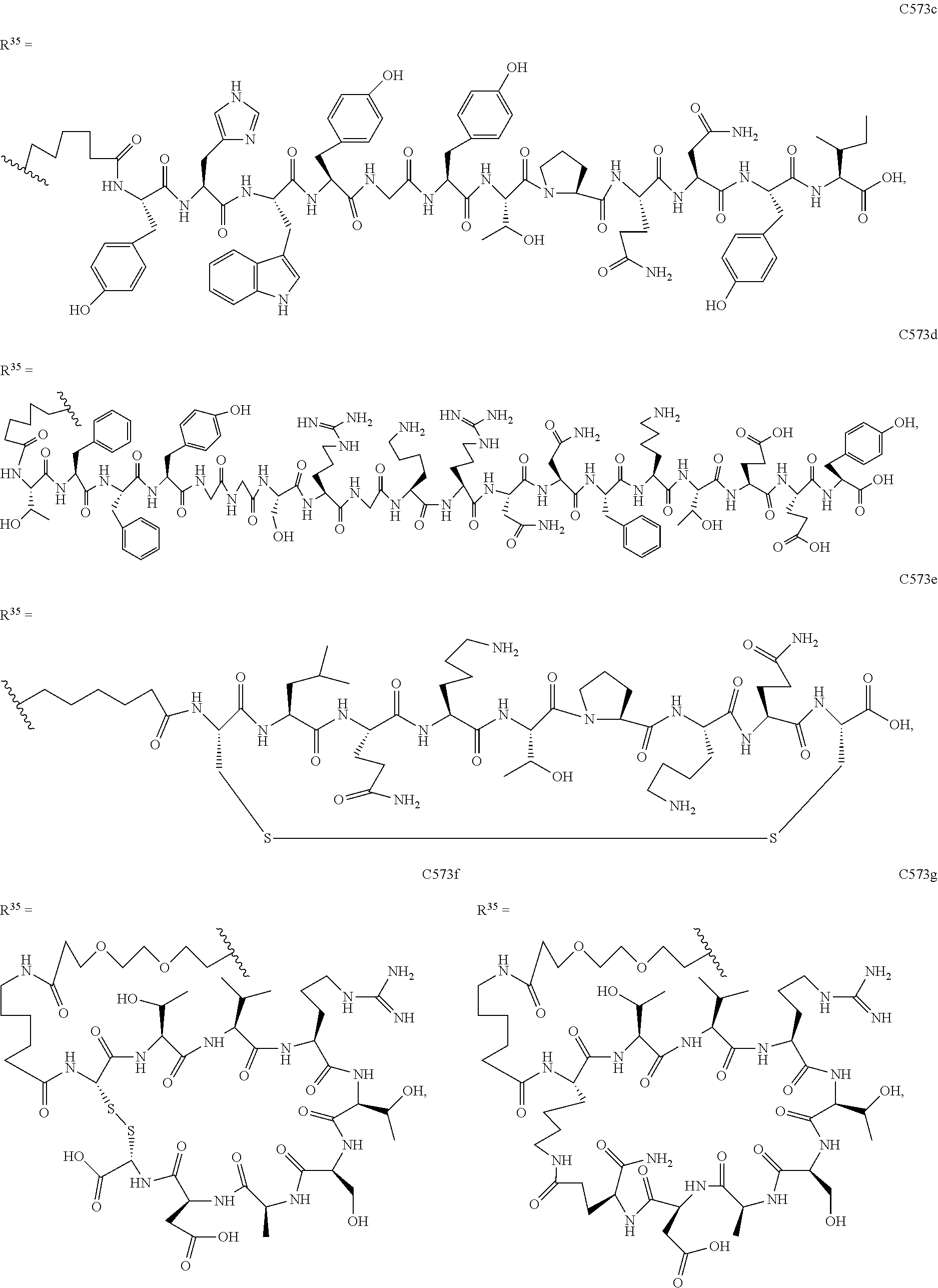

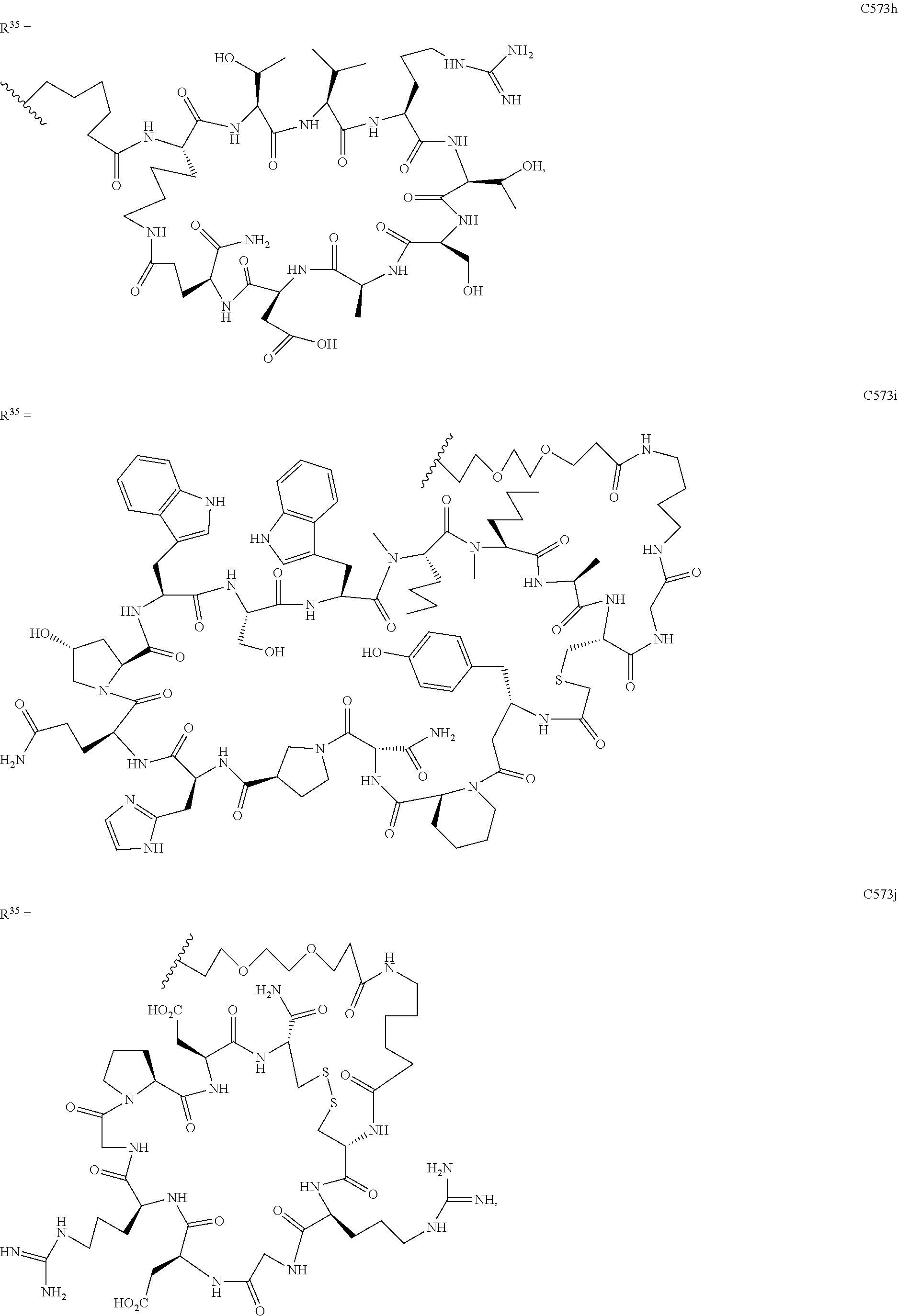

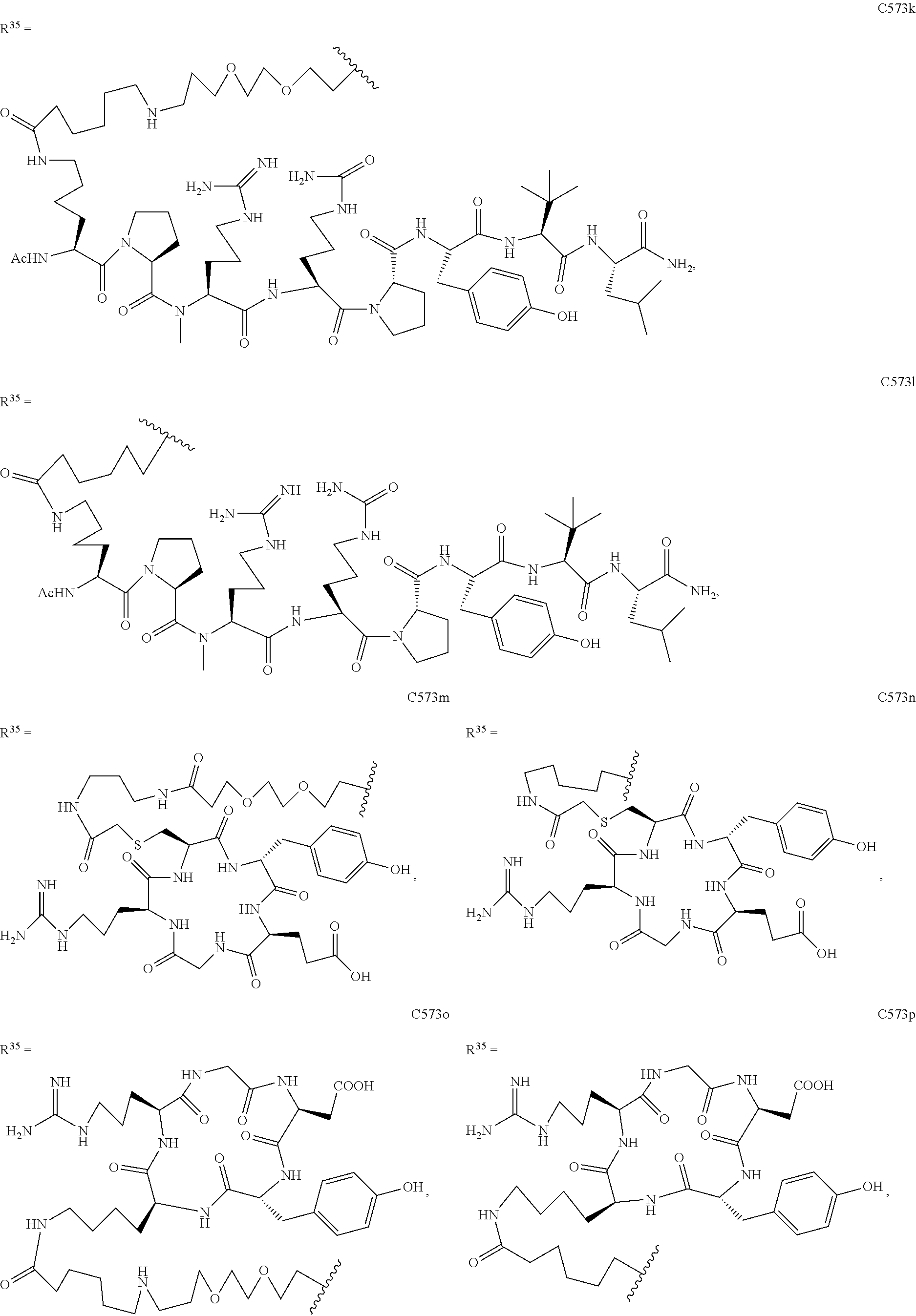

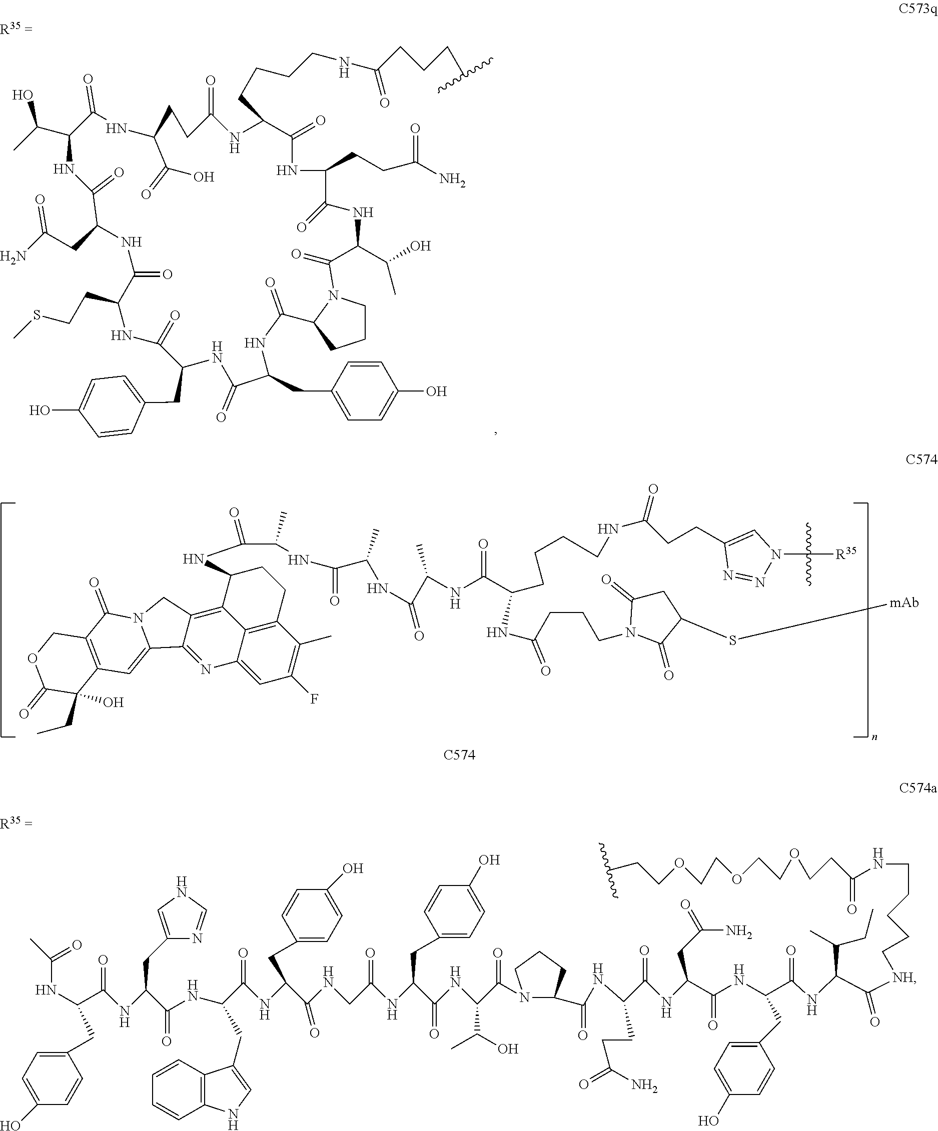

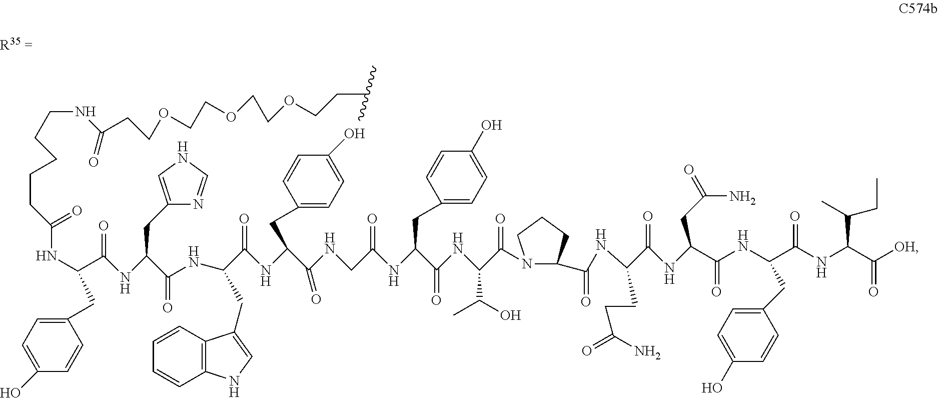 [SMILES: *CCOCCOCCOCCC(=O)NCCCCCC(=O)N[C@@H](Cc1ccc(O)cc1)C(=O)N[C@@H](Cc1c[nH]cn1)C(=O)N[C@@H](Cc1c[nH]c2ccccc12)C(=O)N[C@@H](Cc1ccc(O)cc1)C(=O)NCC(=O)N[C@@H](Cc1ccc(O)cc1)C(=O)N[C@H](C(=O)N1CCC[C@H]1C(=O)N[C@@H](CCC(N)=O)C(=O)N[C@@H](CC(N)=O)C(=O)N[C@@H](Cc1ccc(O)cc1)C(=O)N[C@H](C(=O)O)C(C)CC)C(C)O]

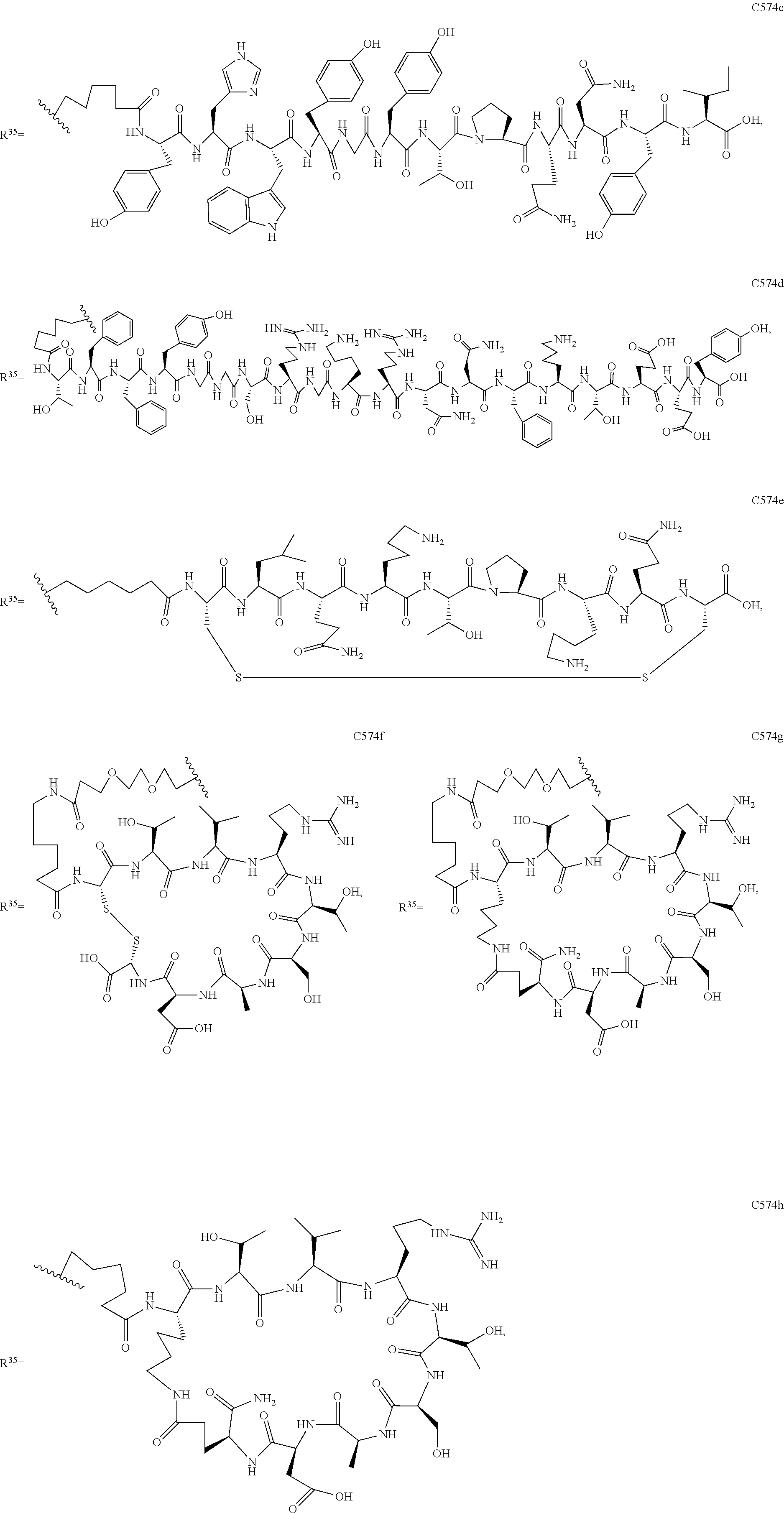

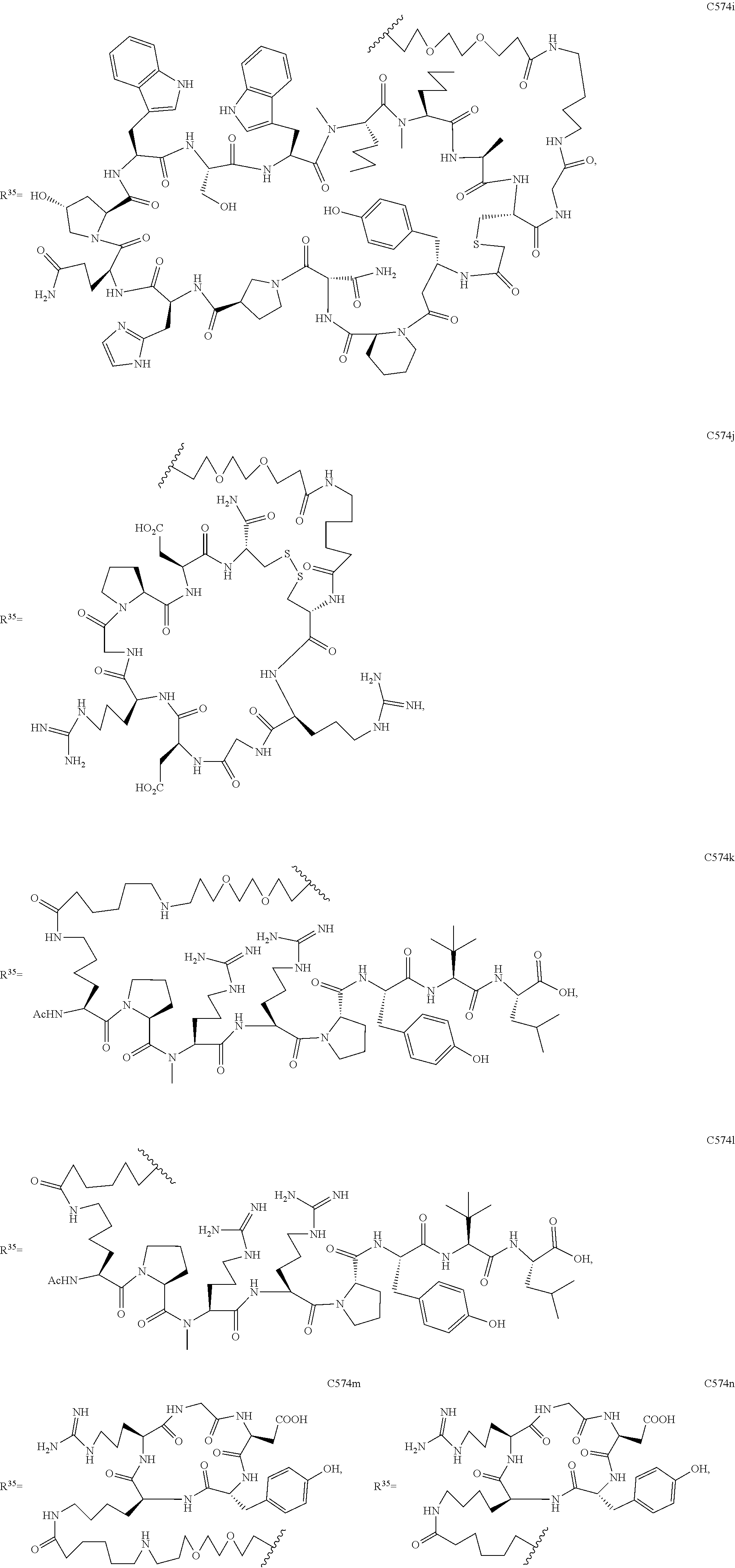

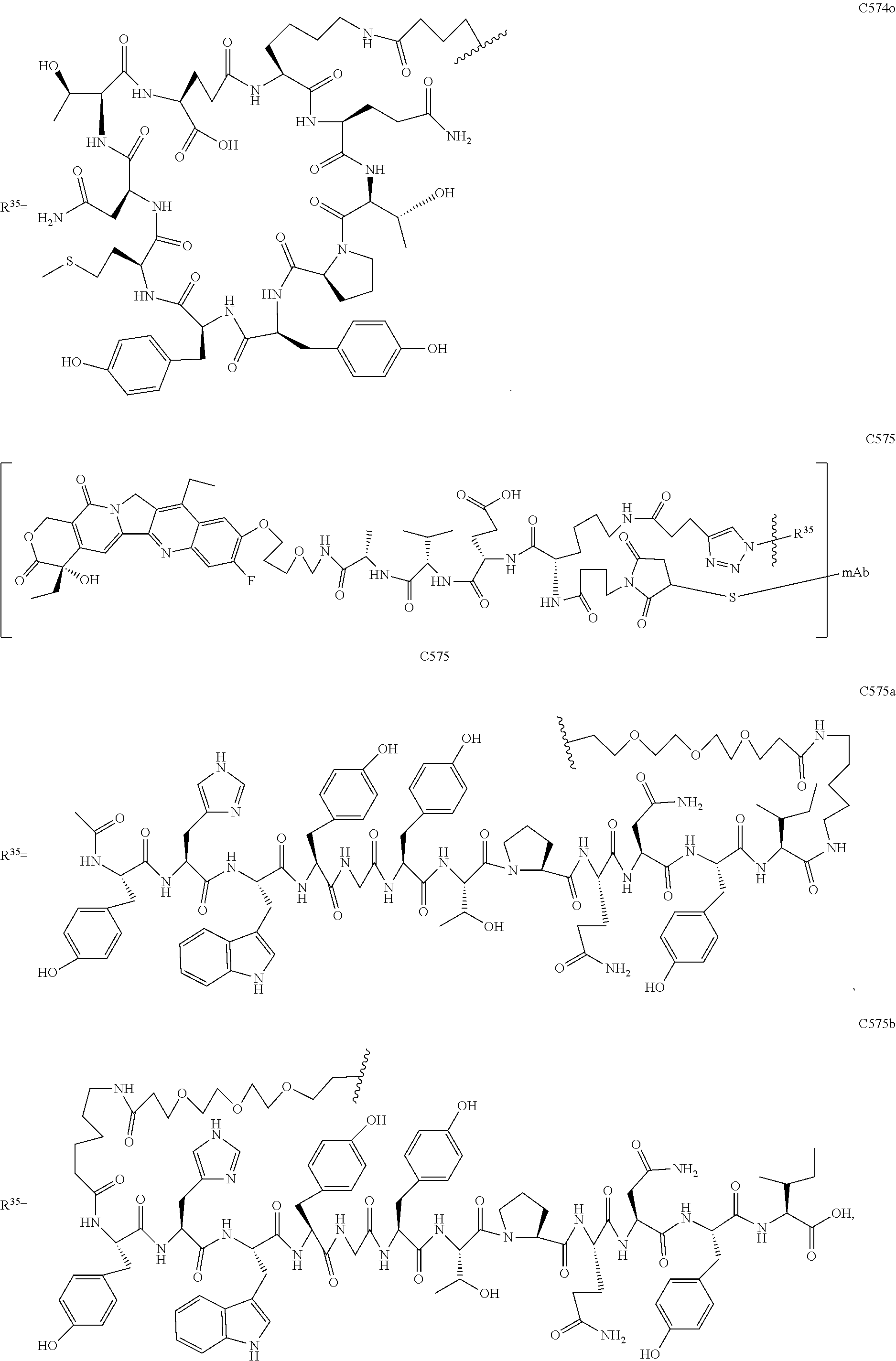

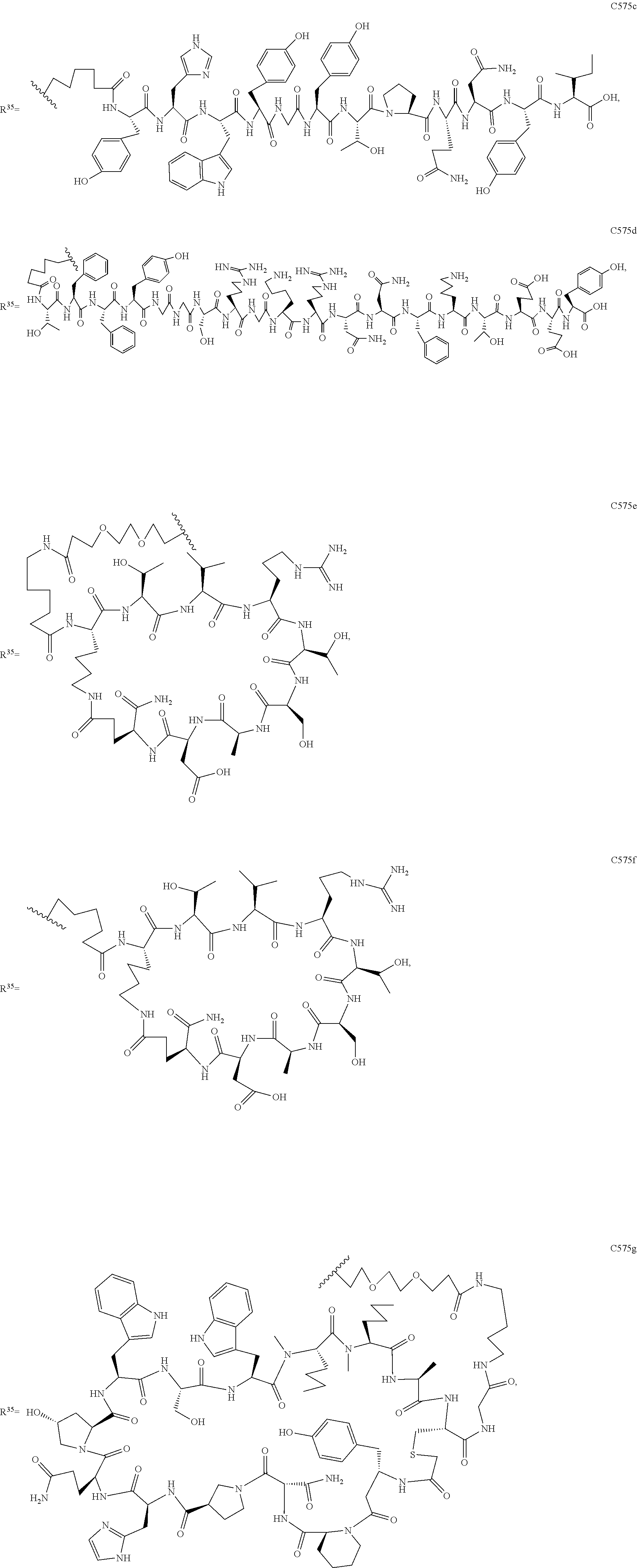

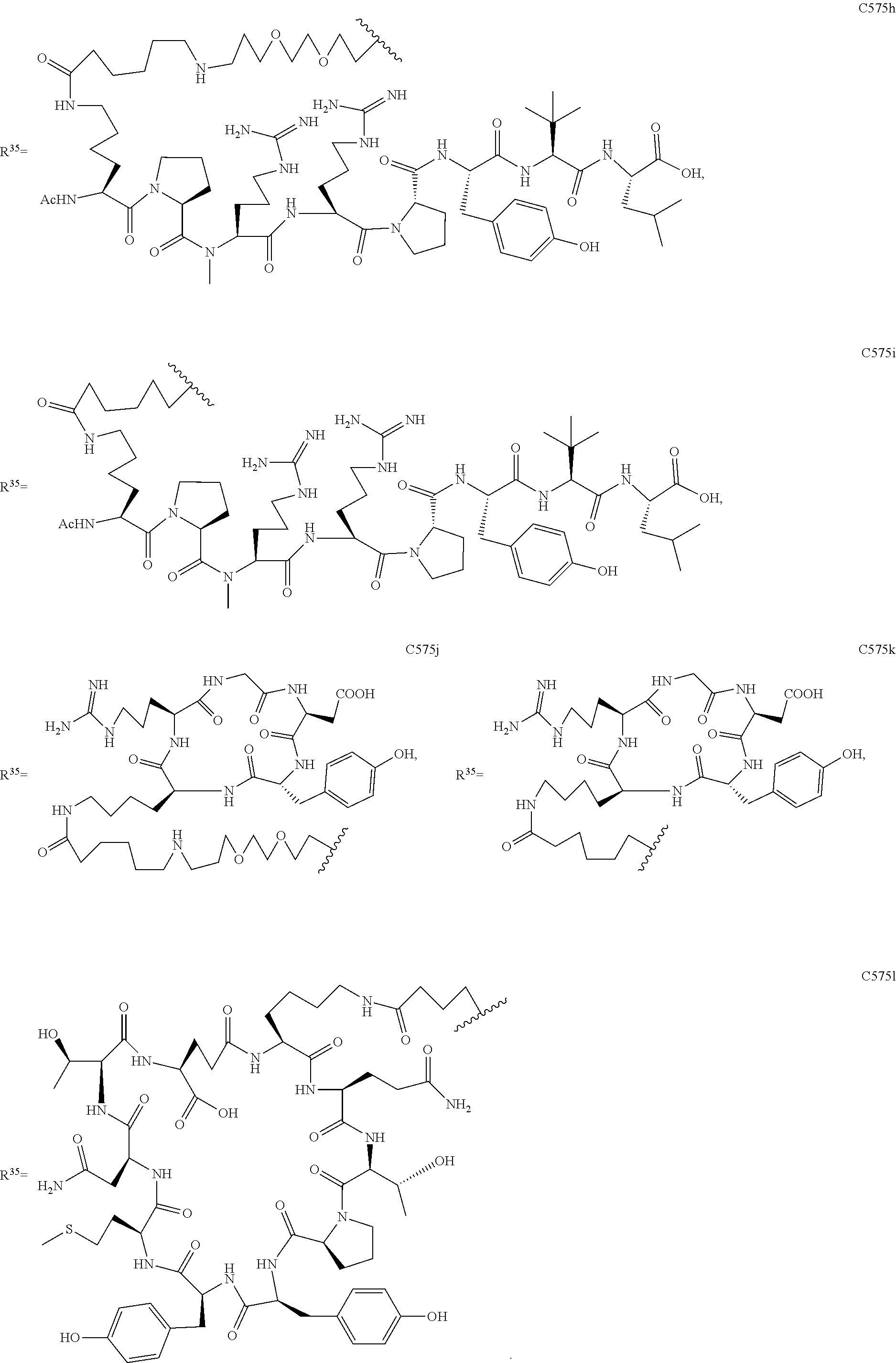

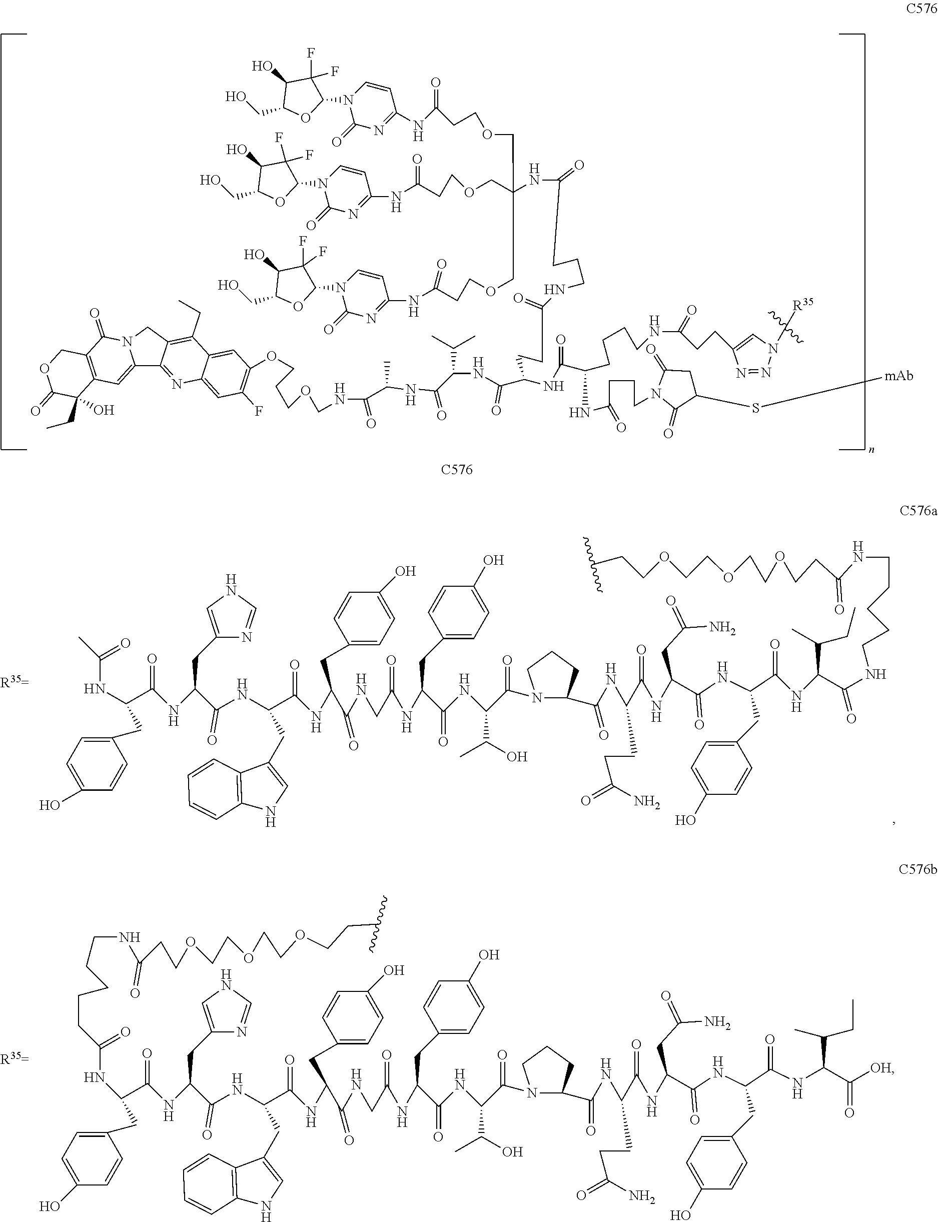

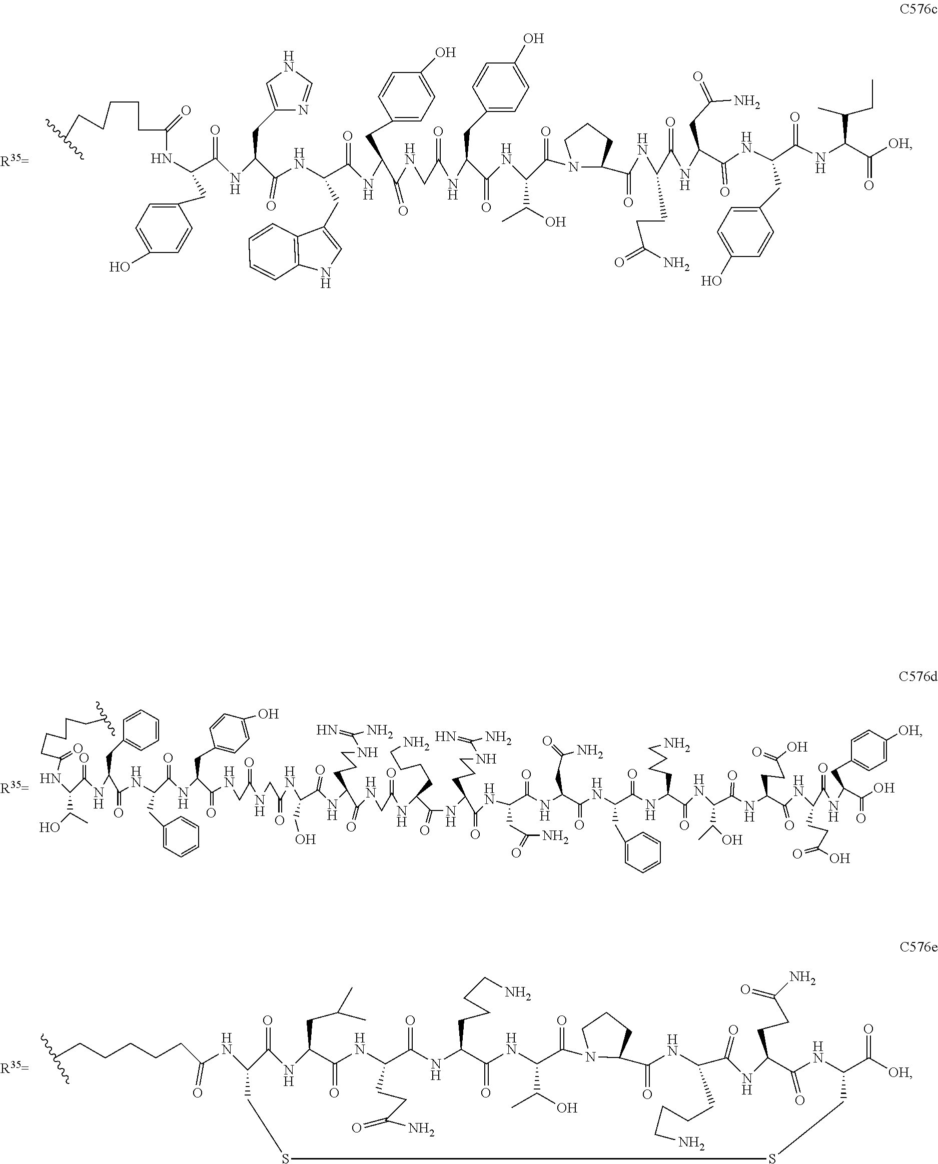

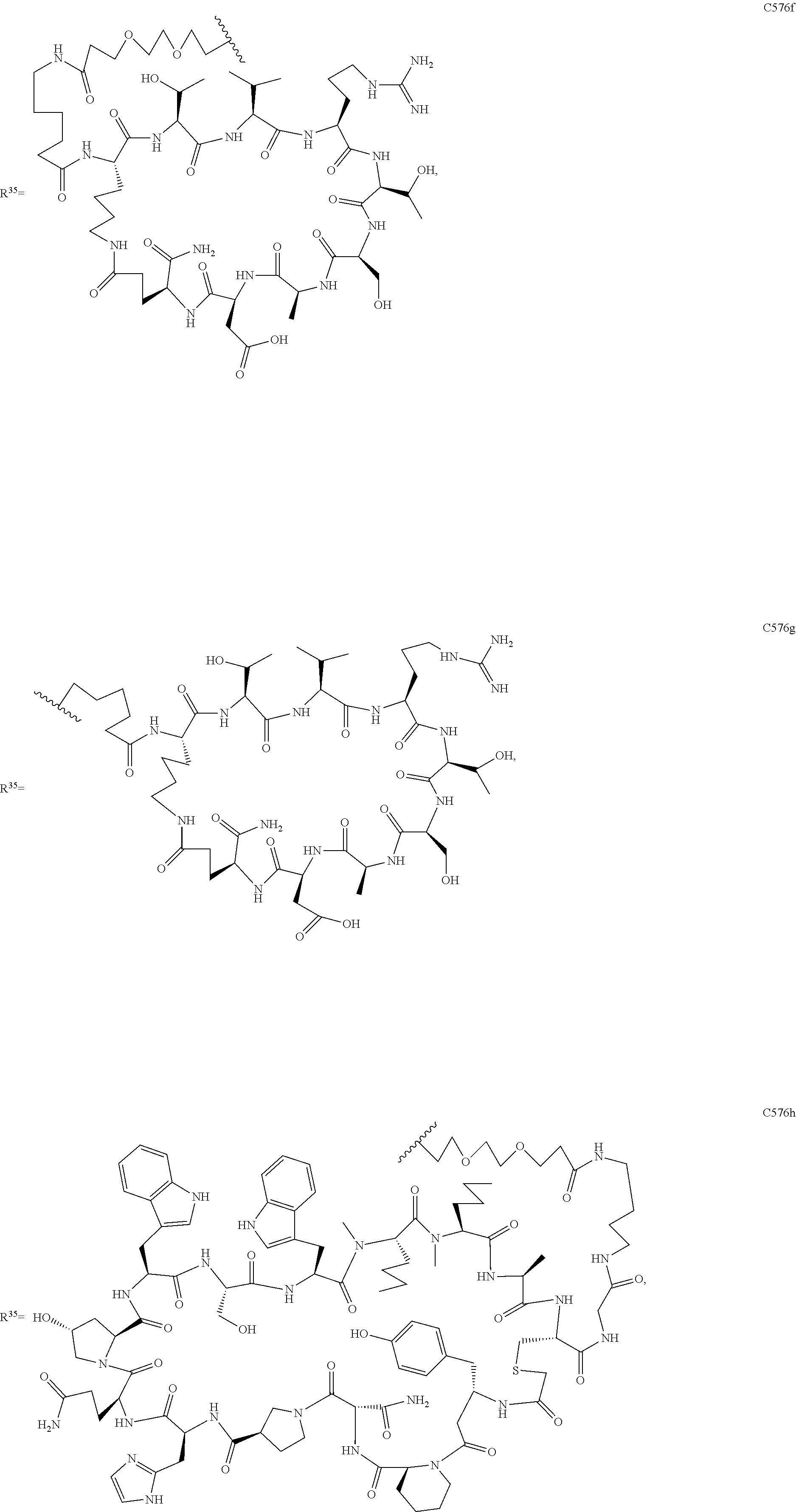

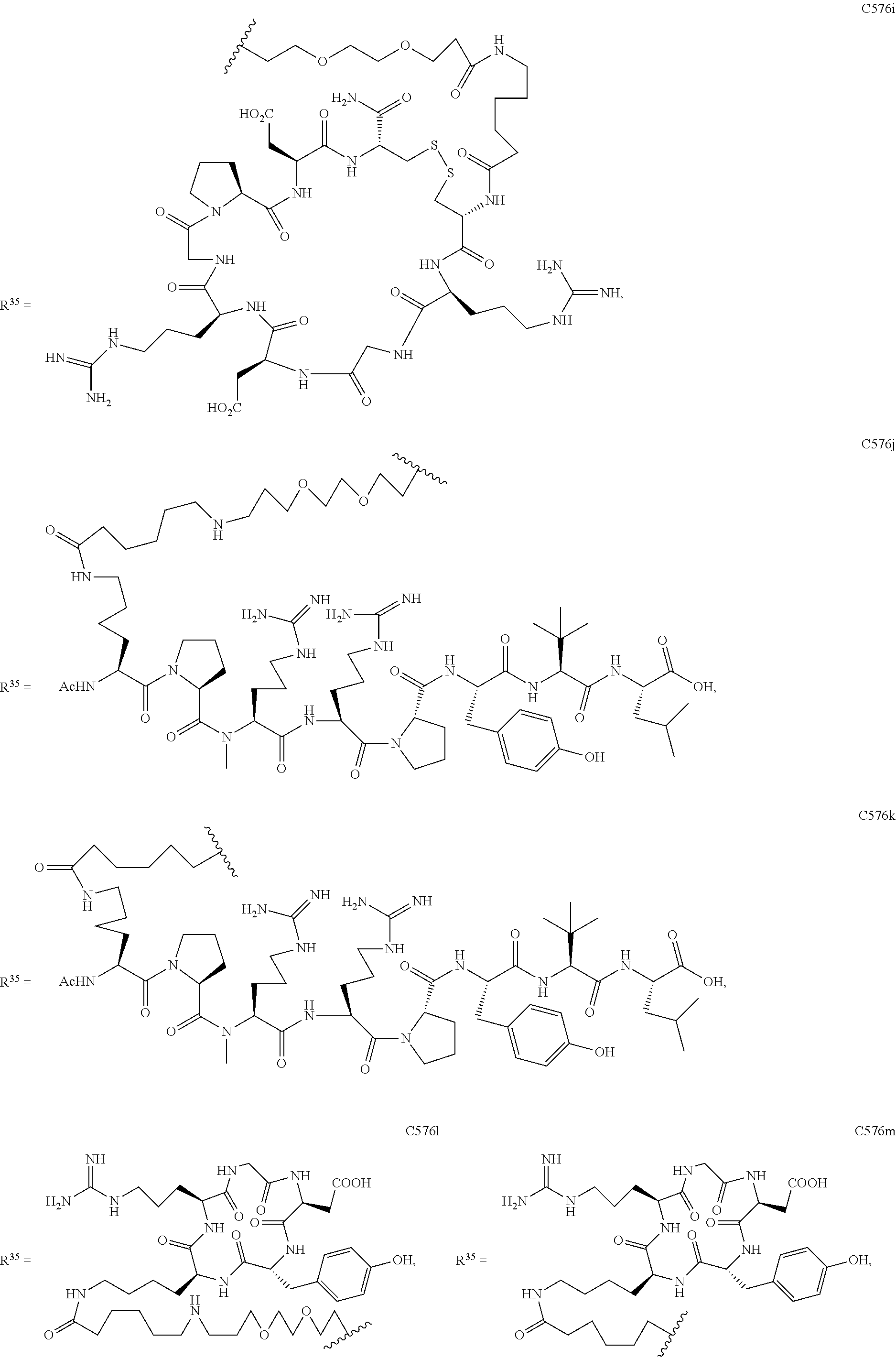

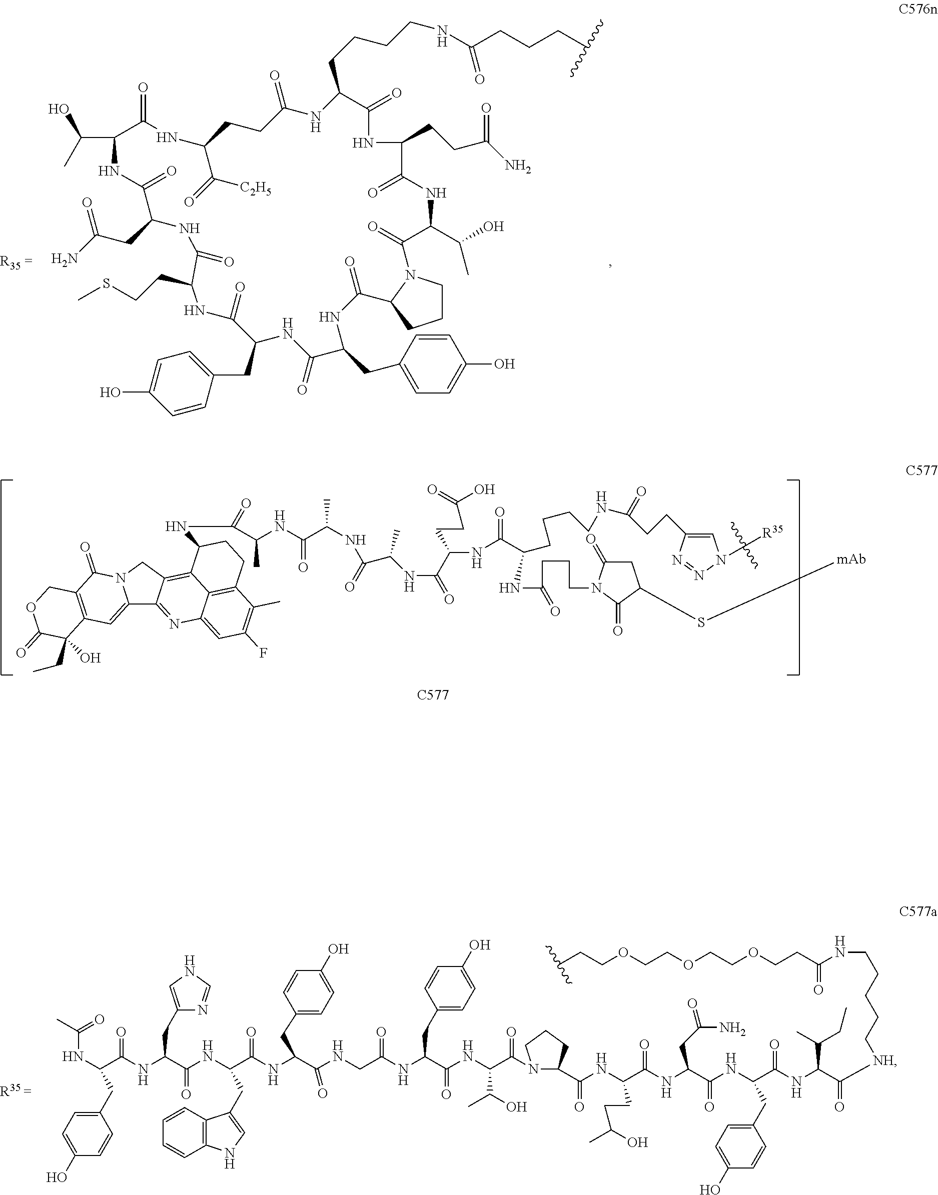

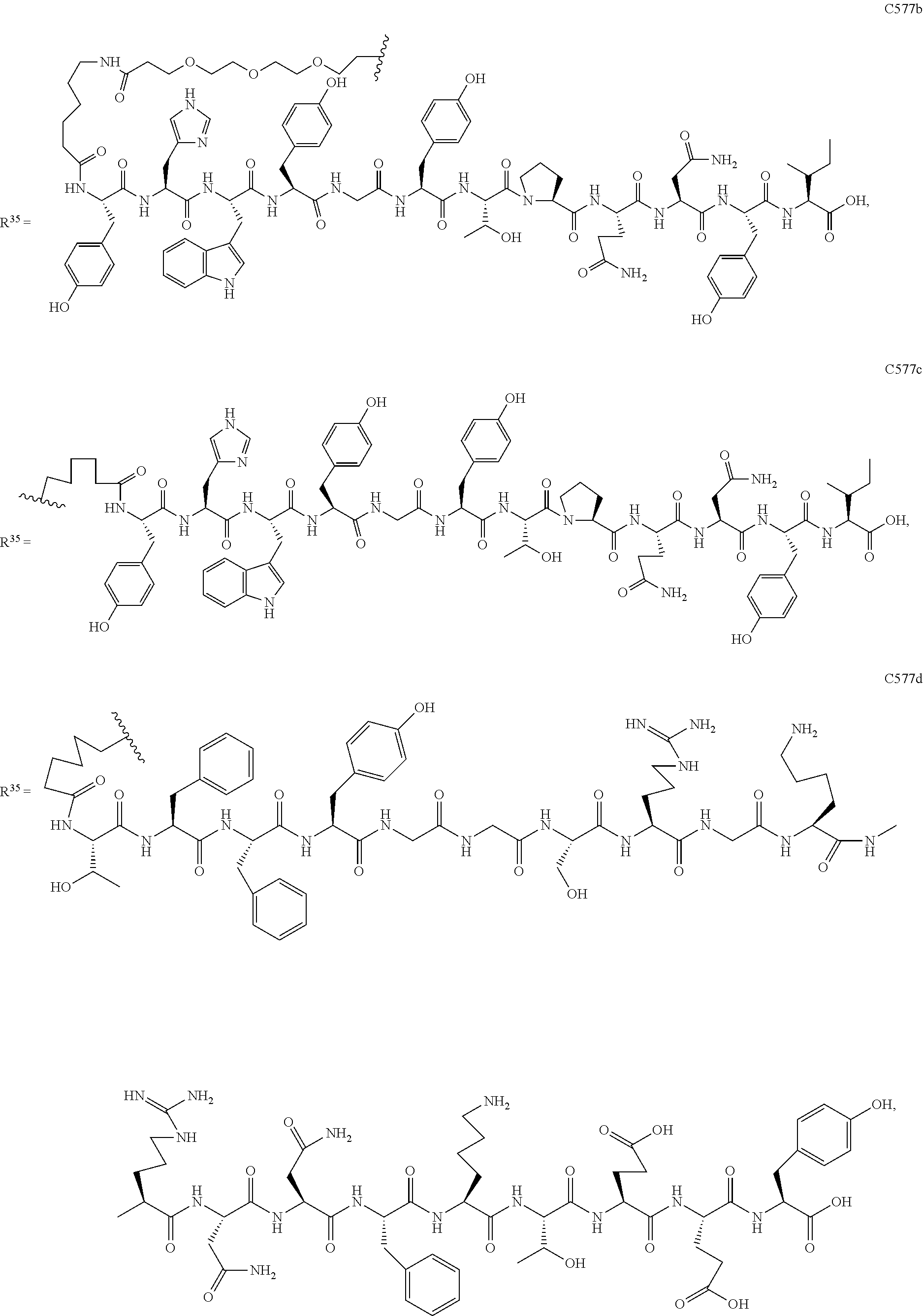

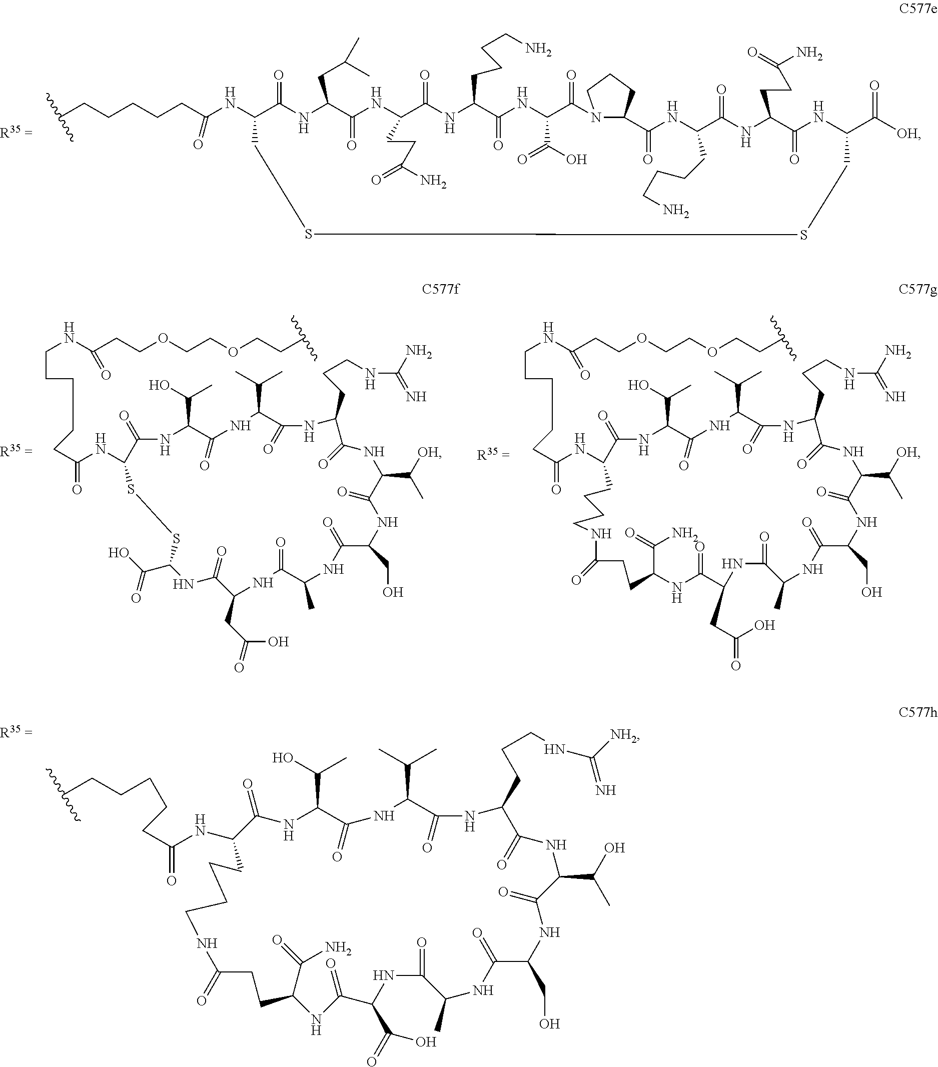

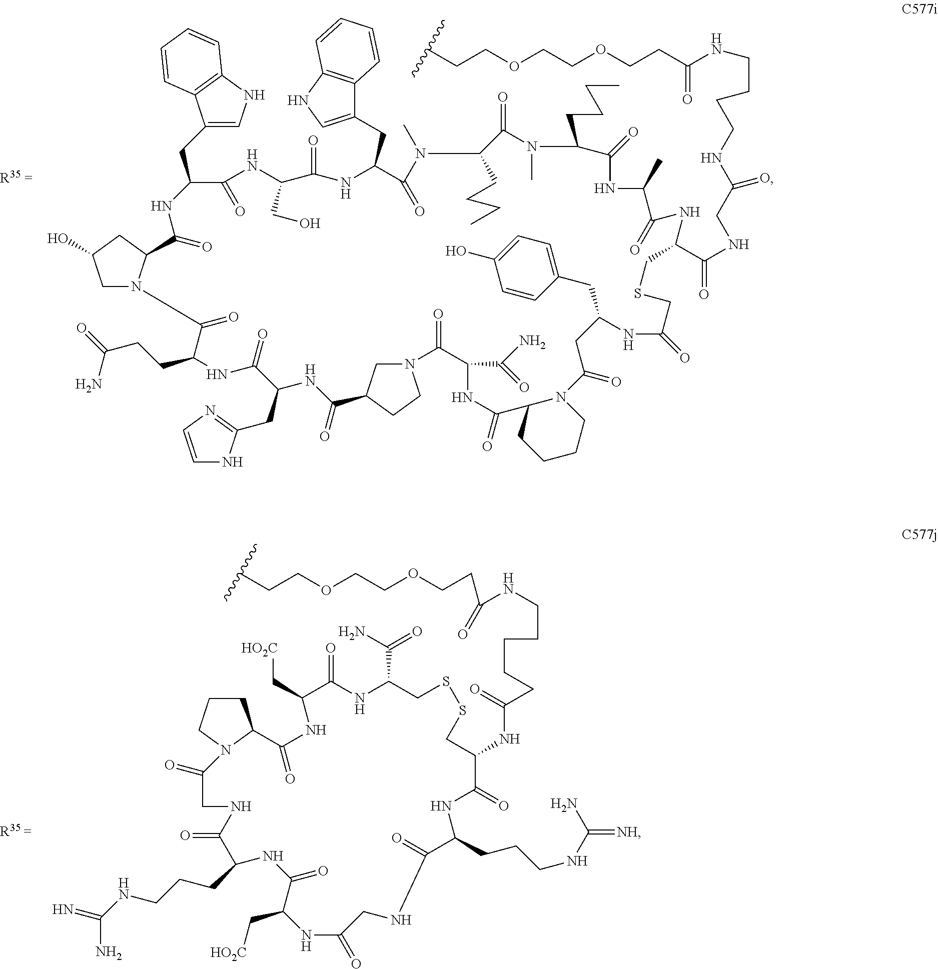

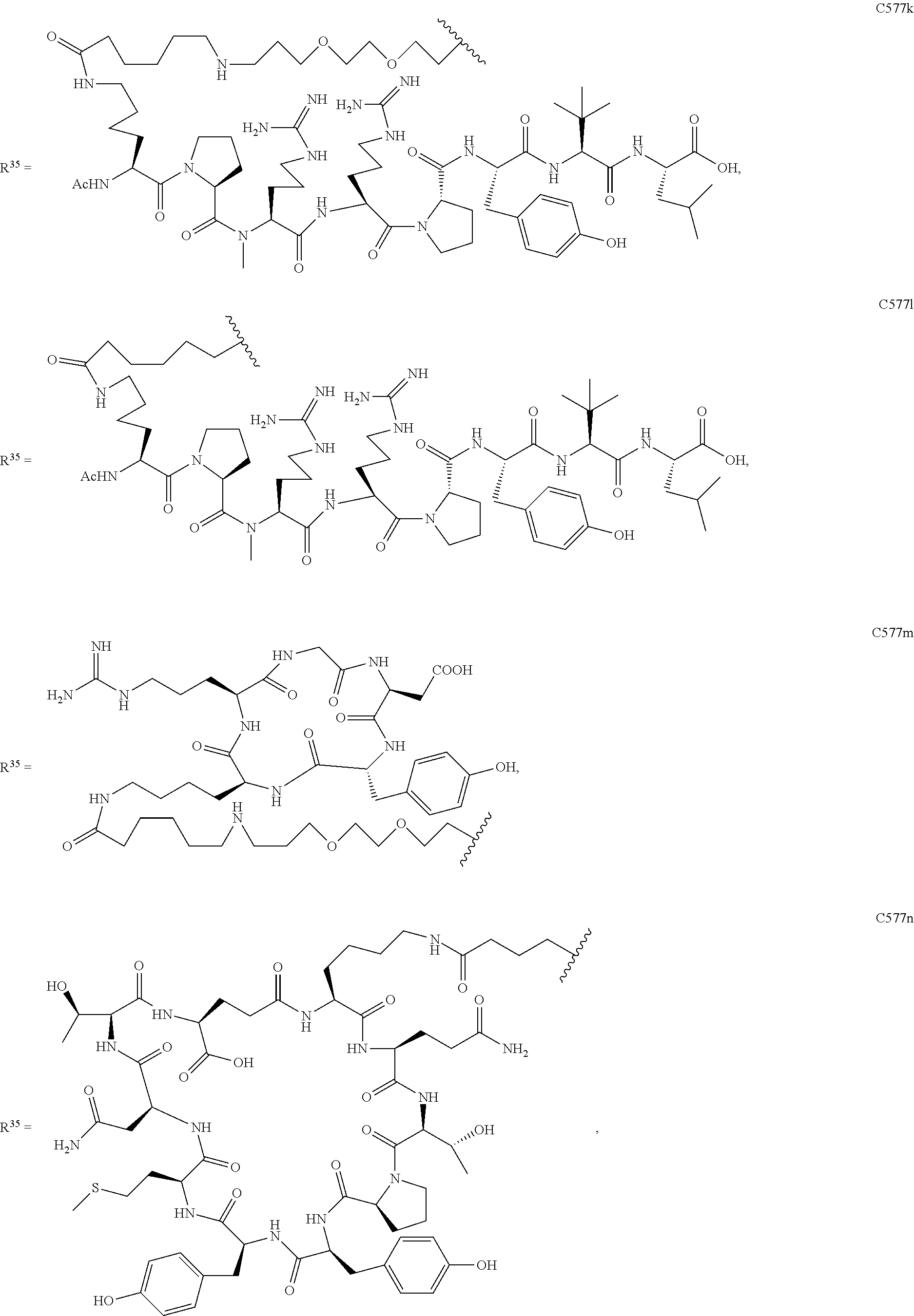

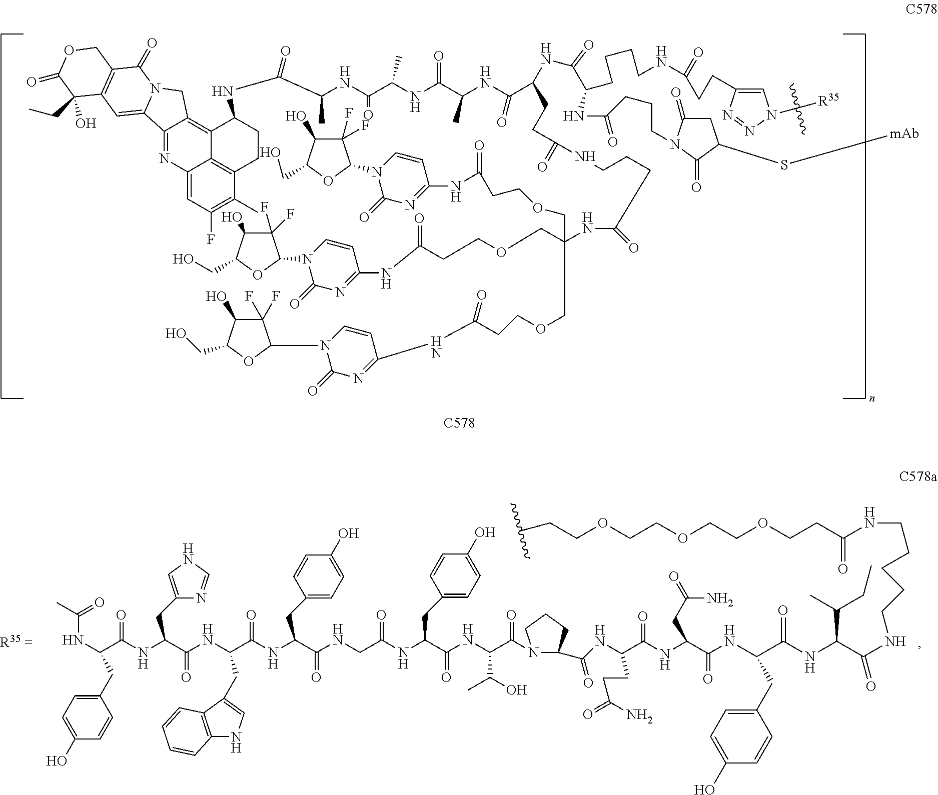

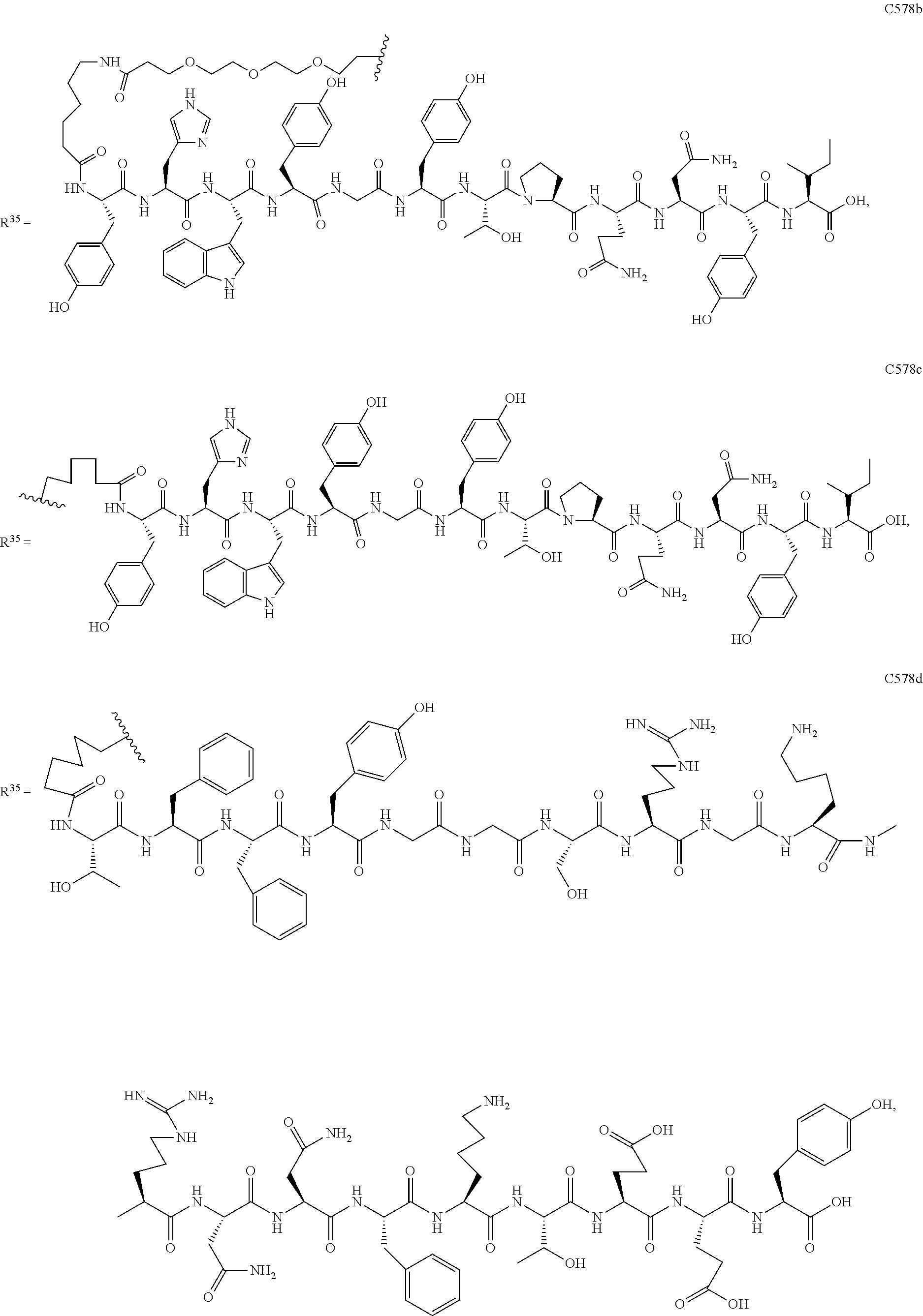

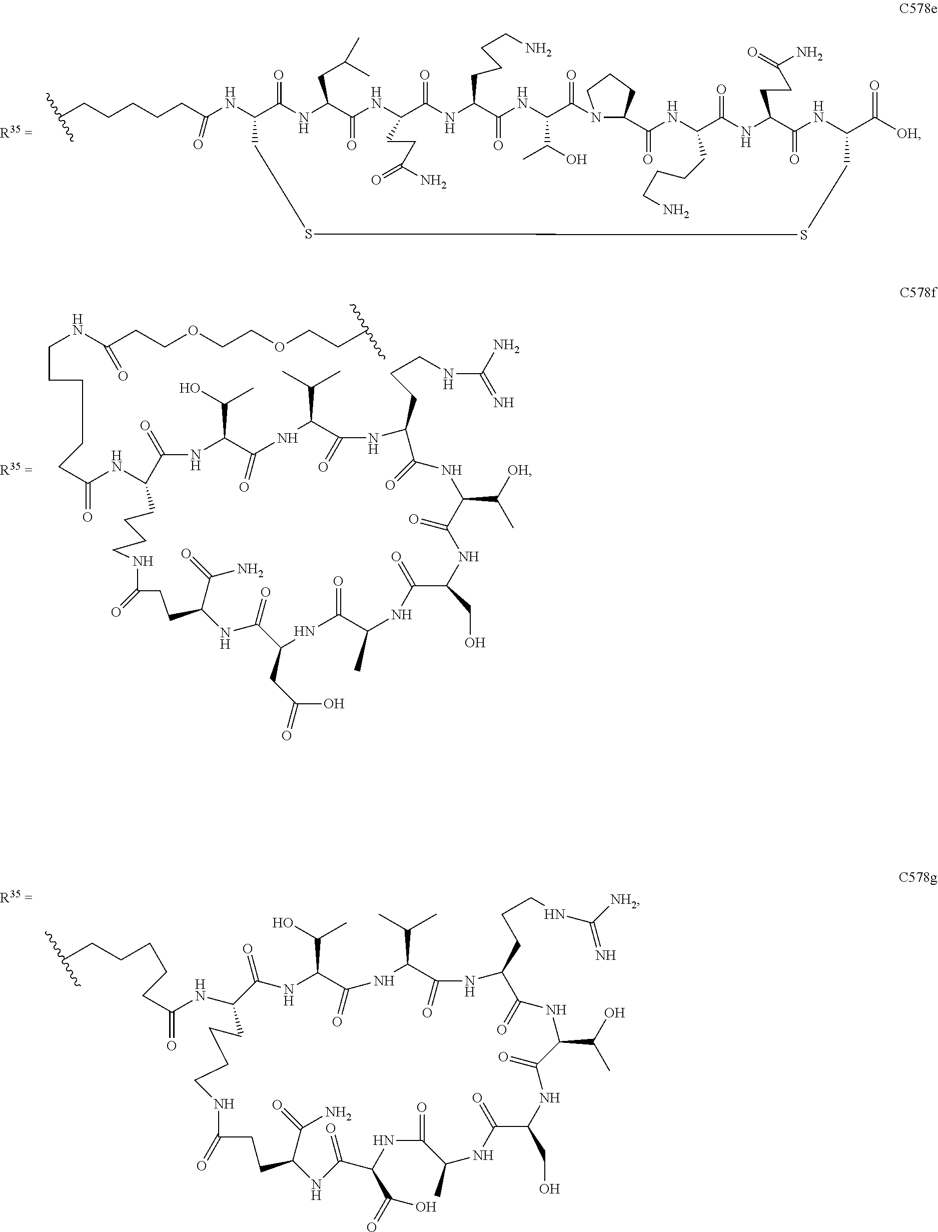

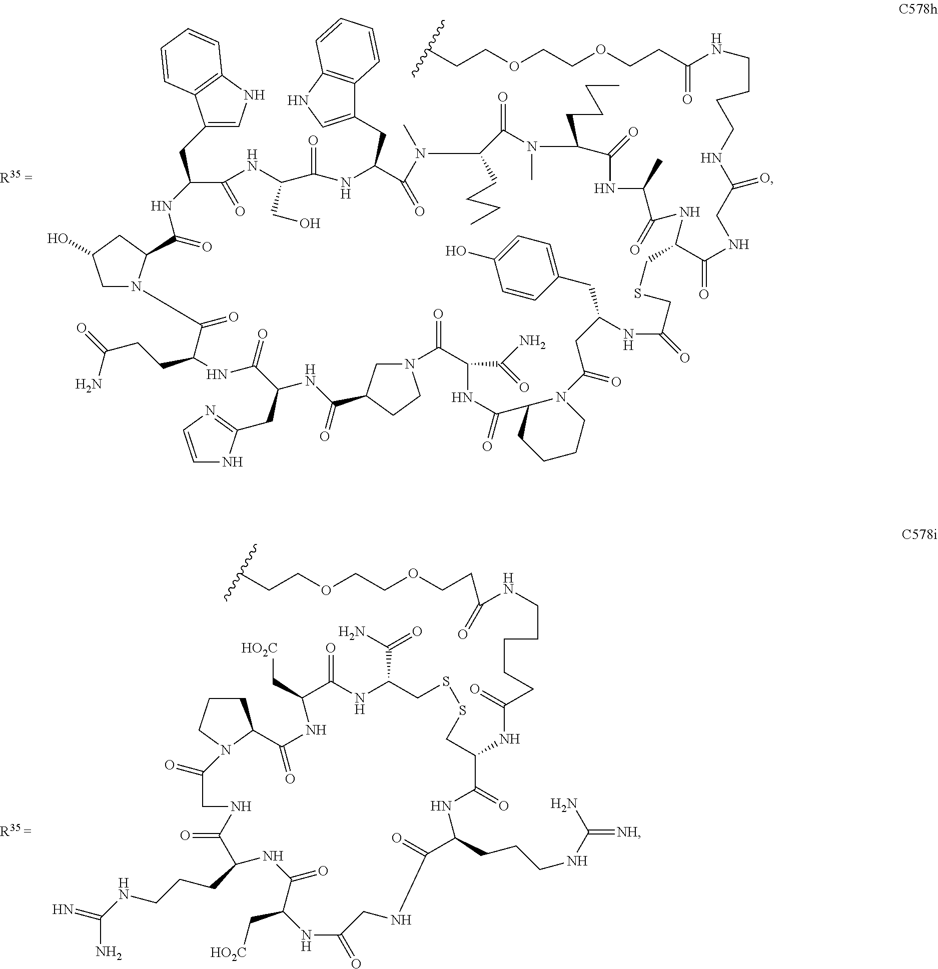

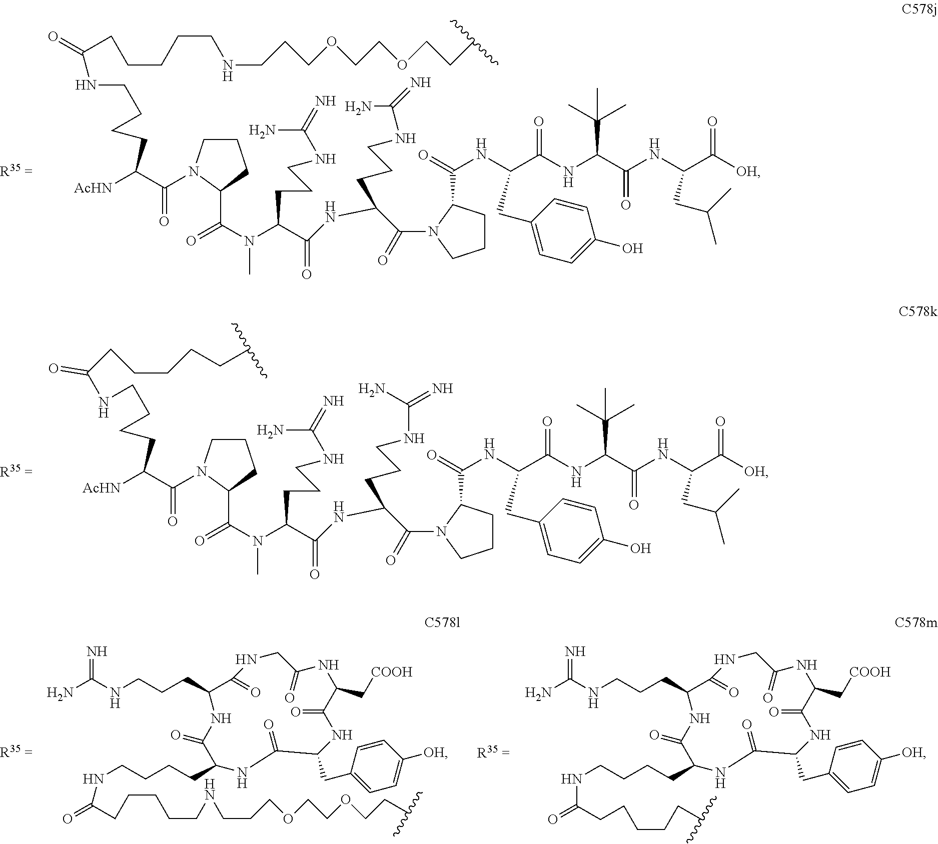

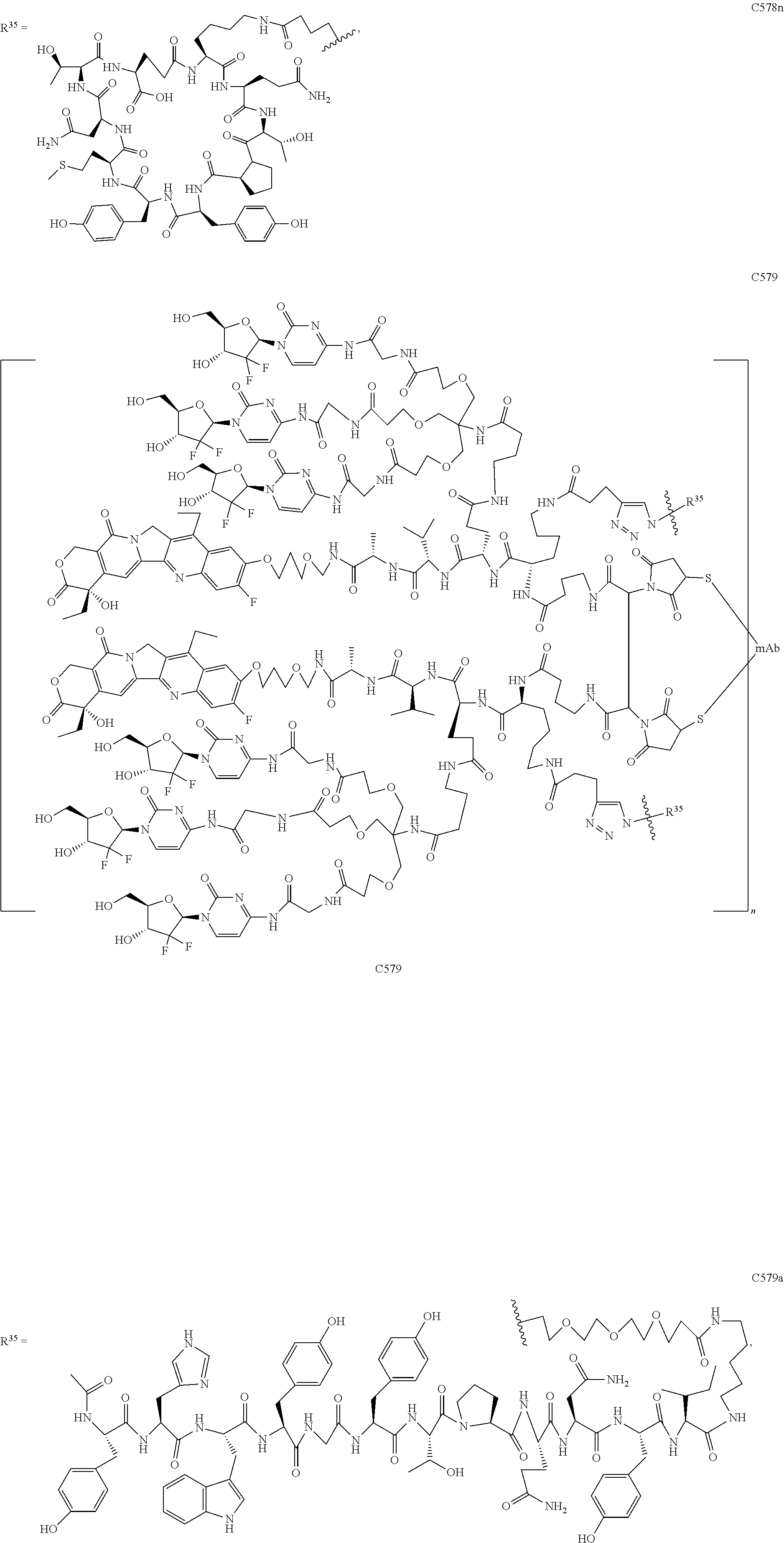

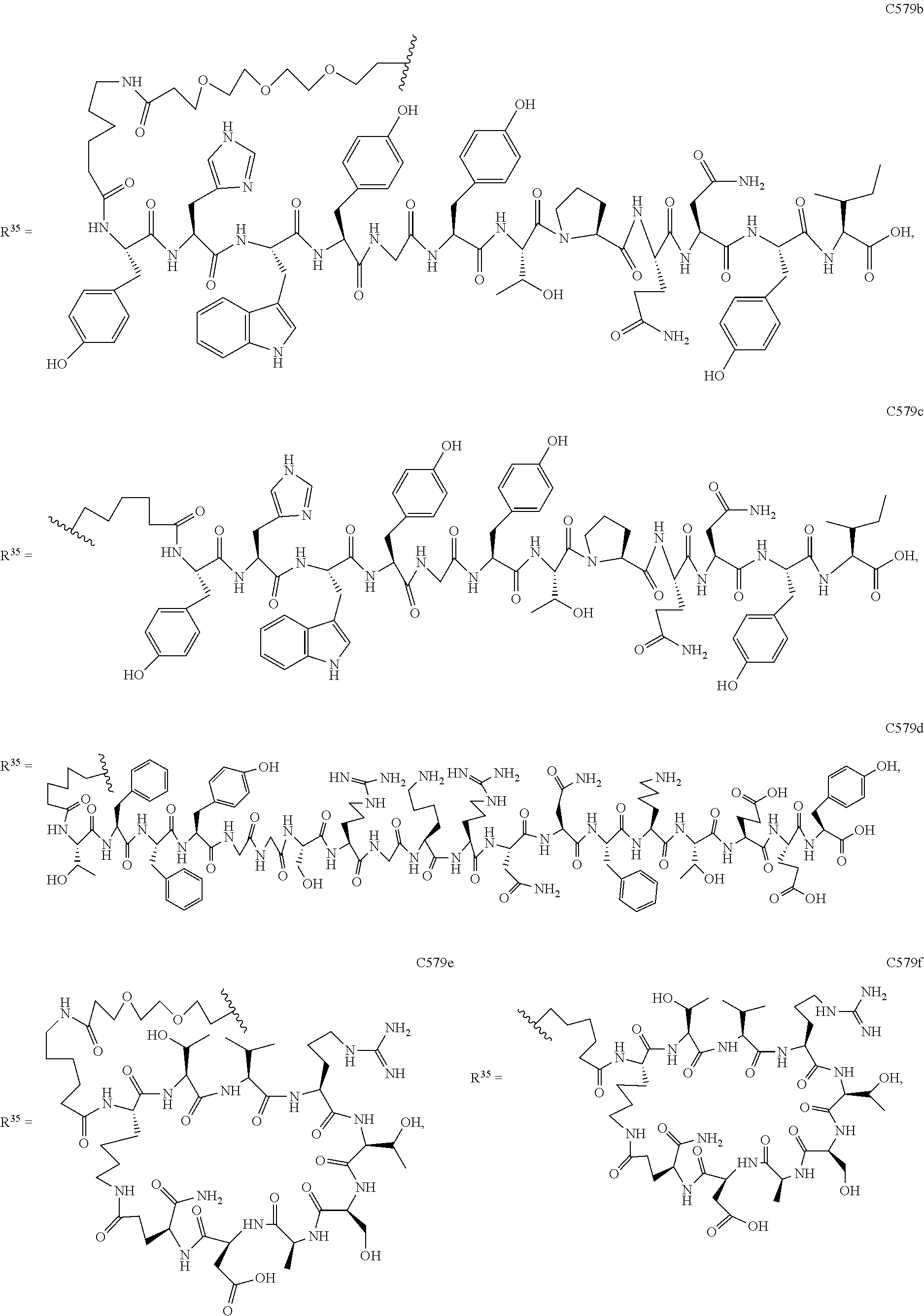

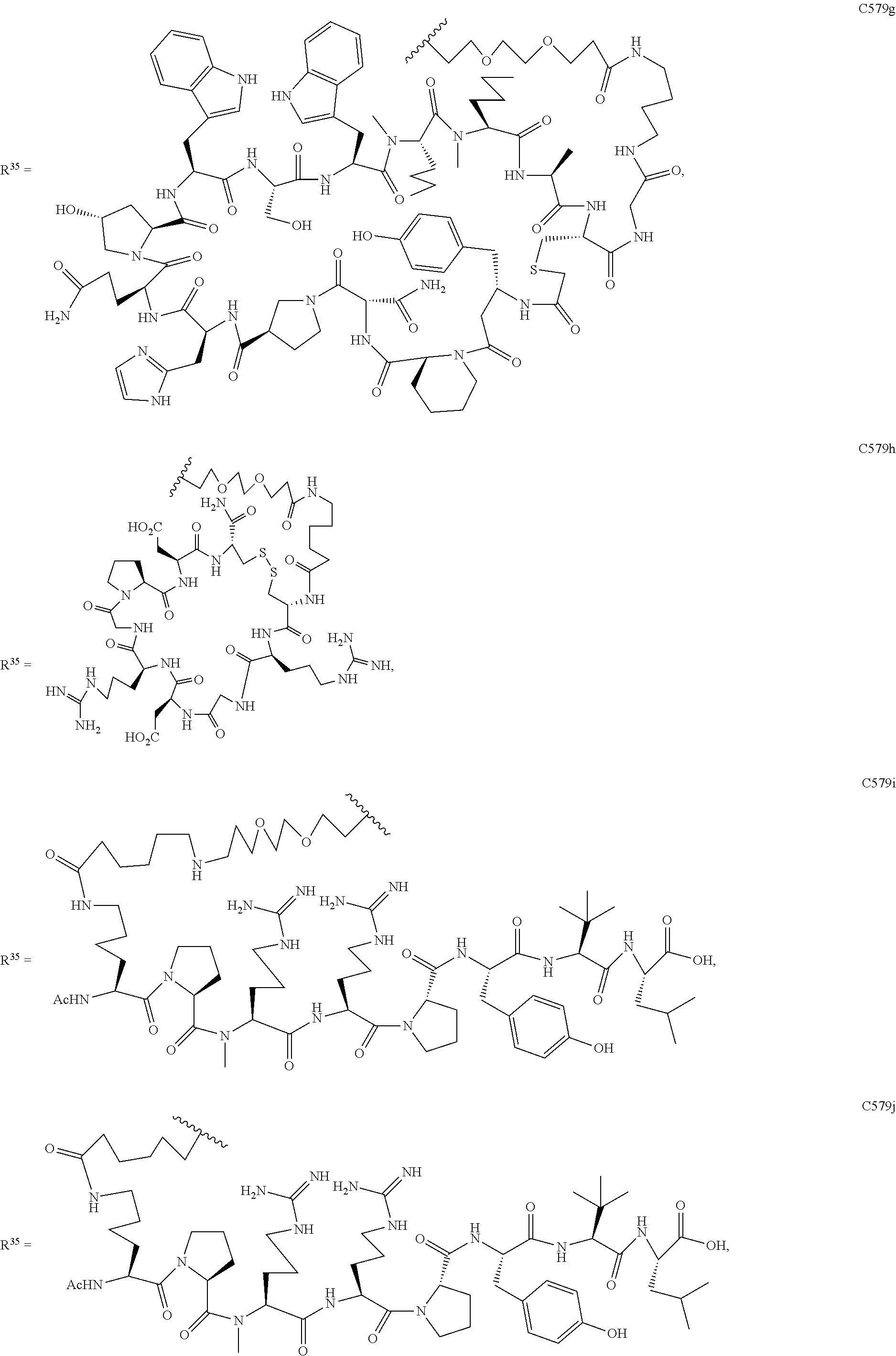

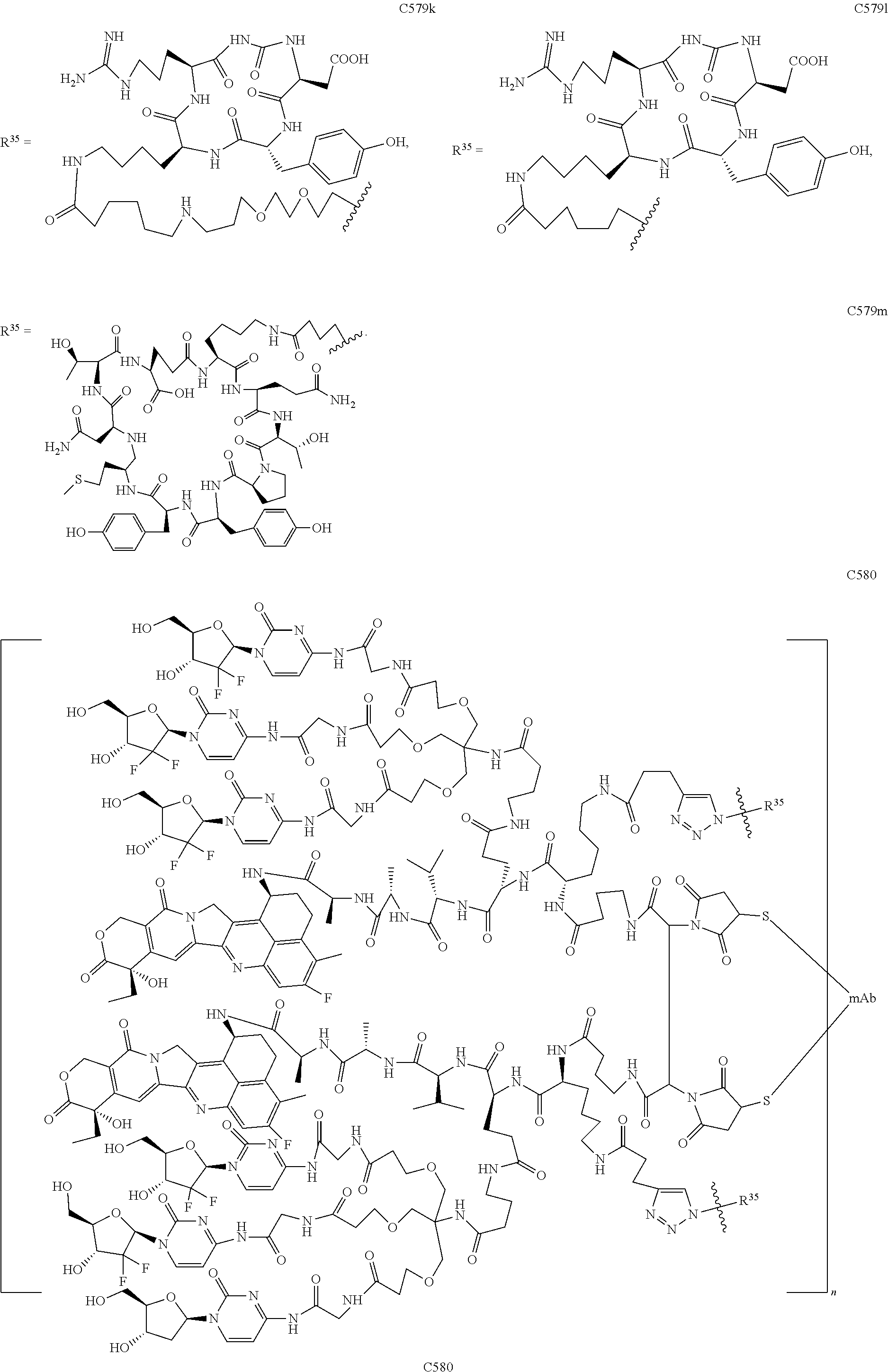

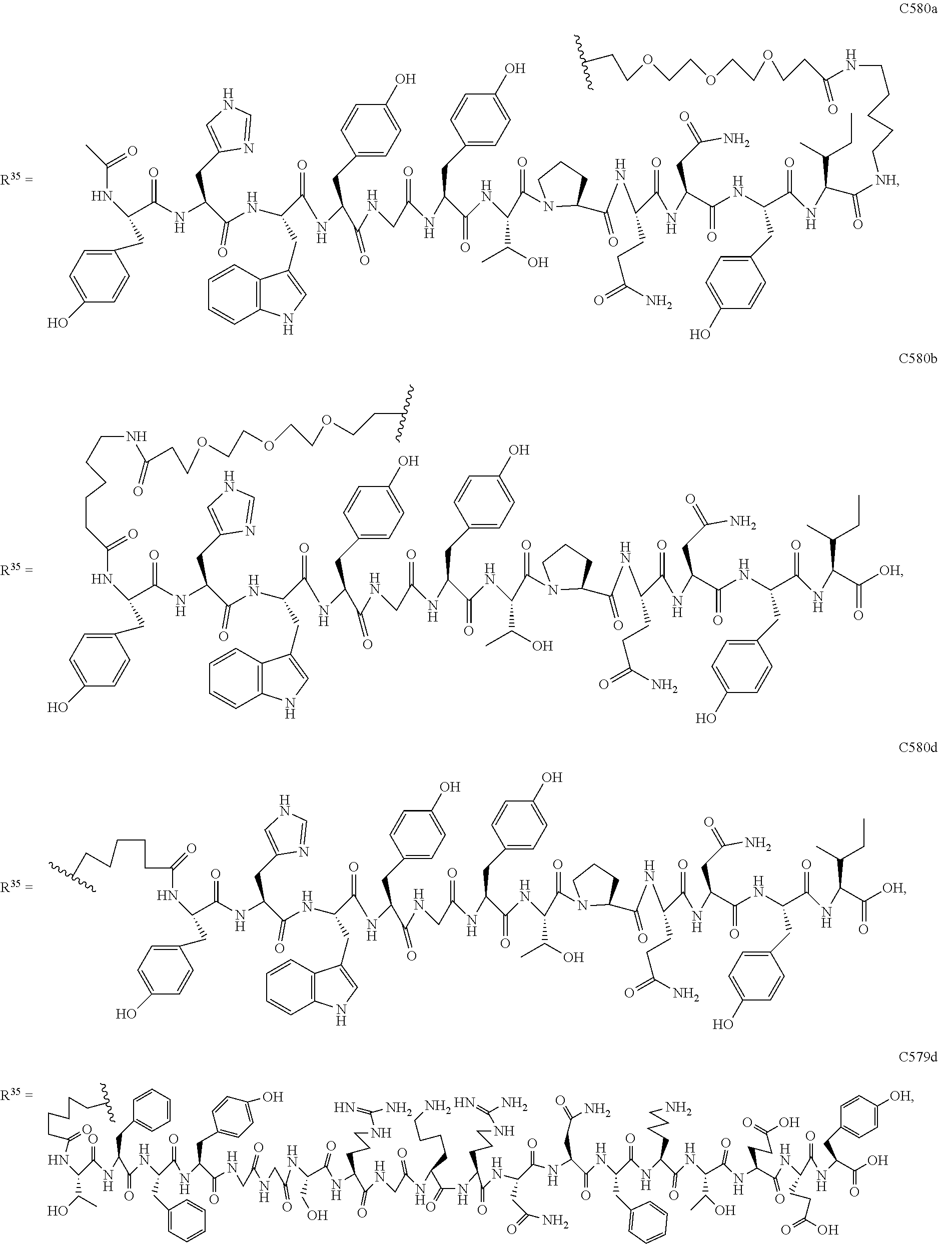

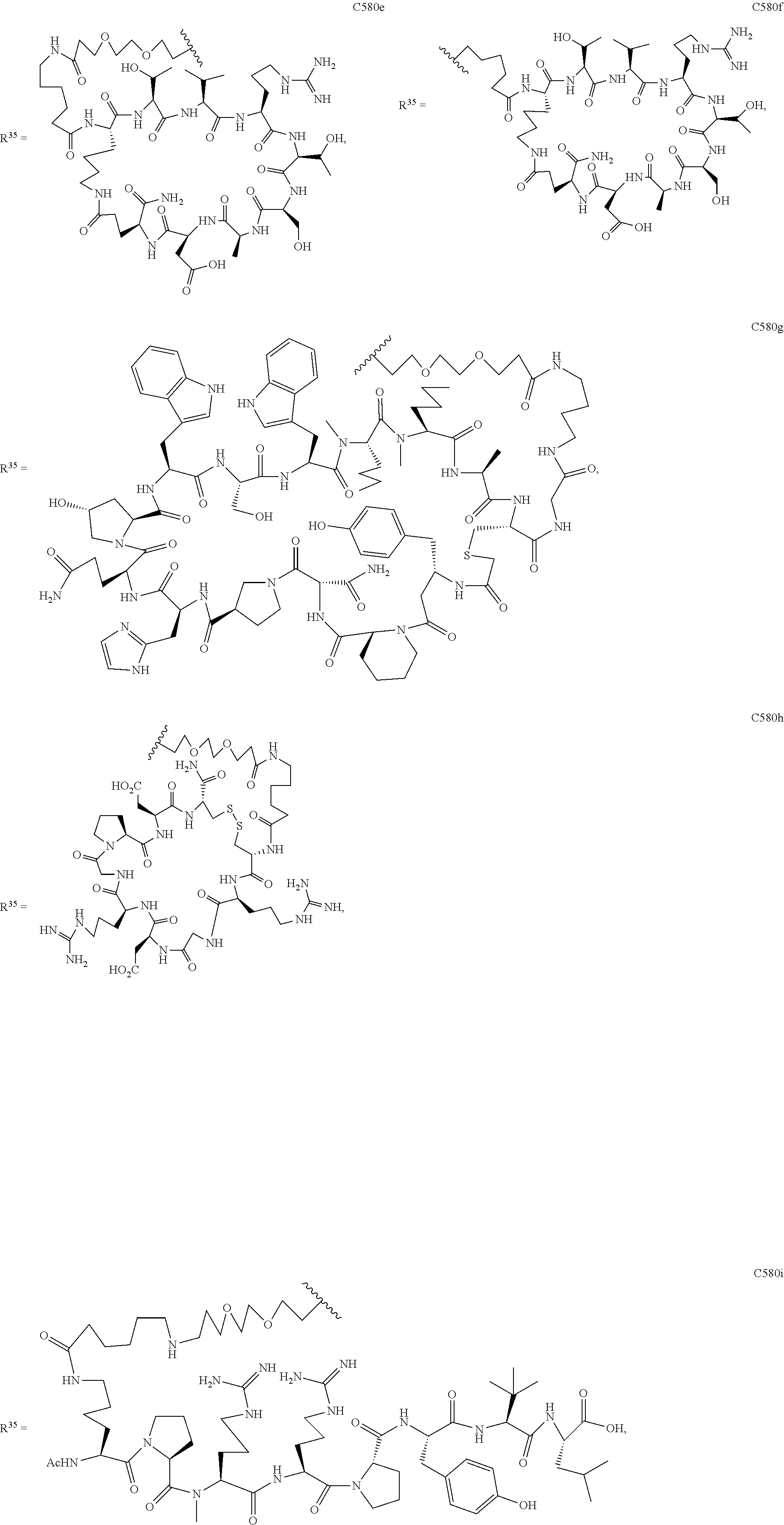

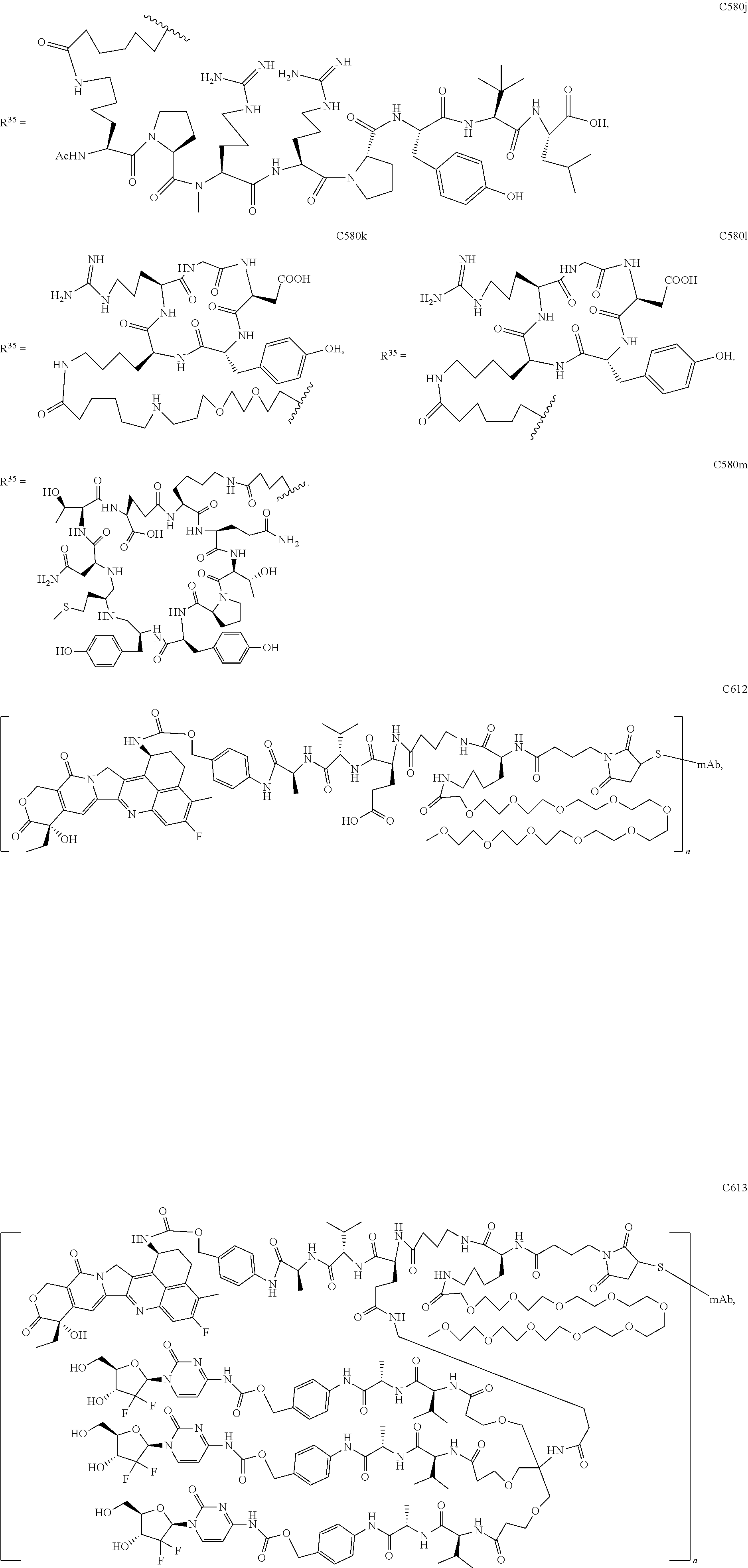

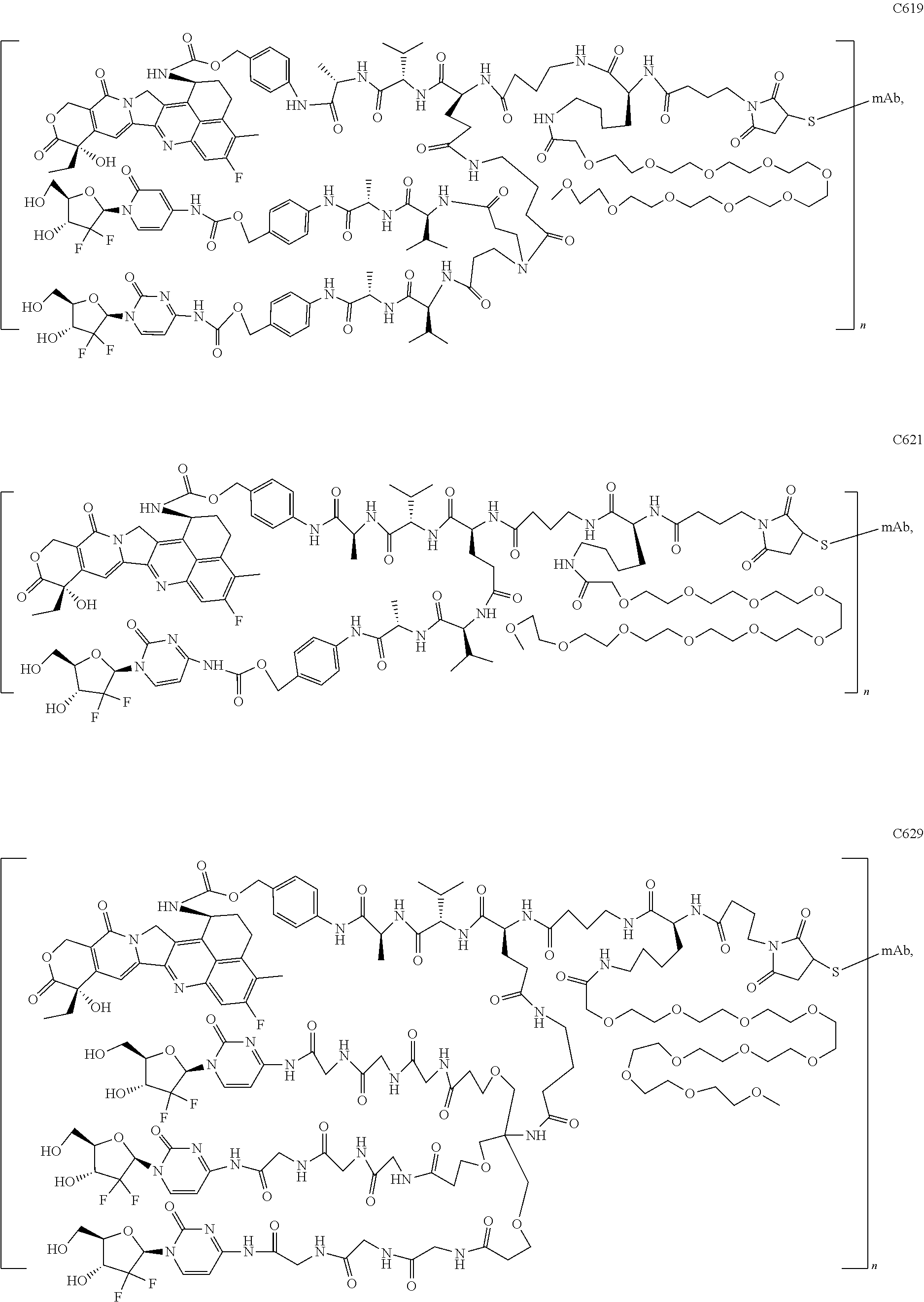

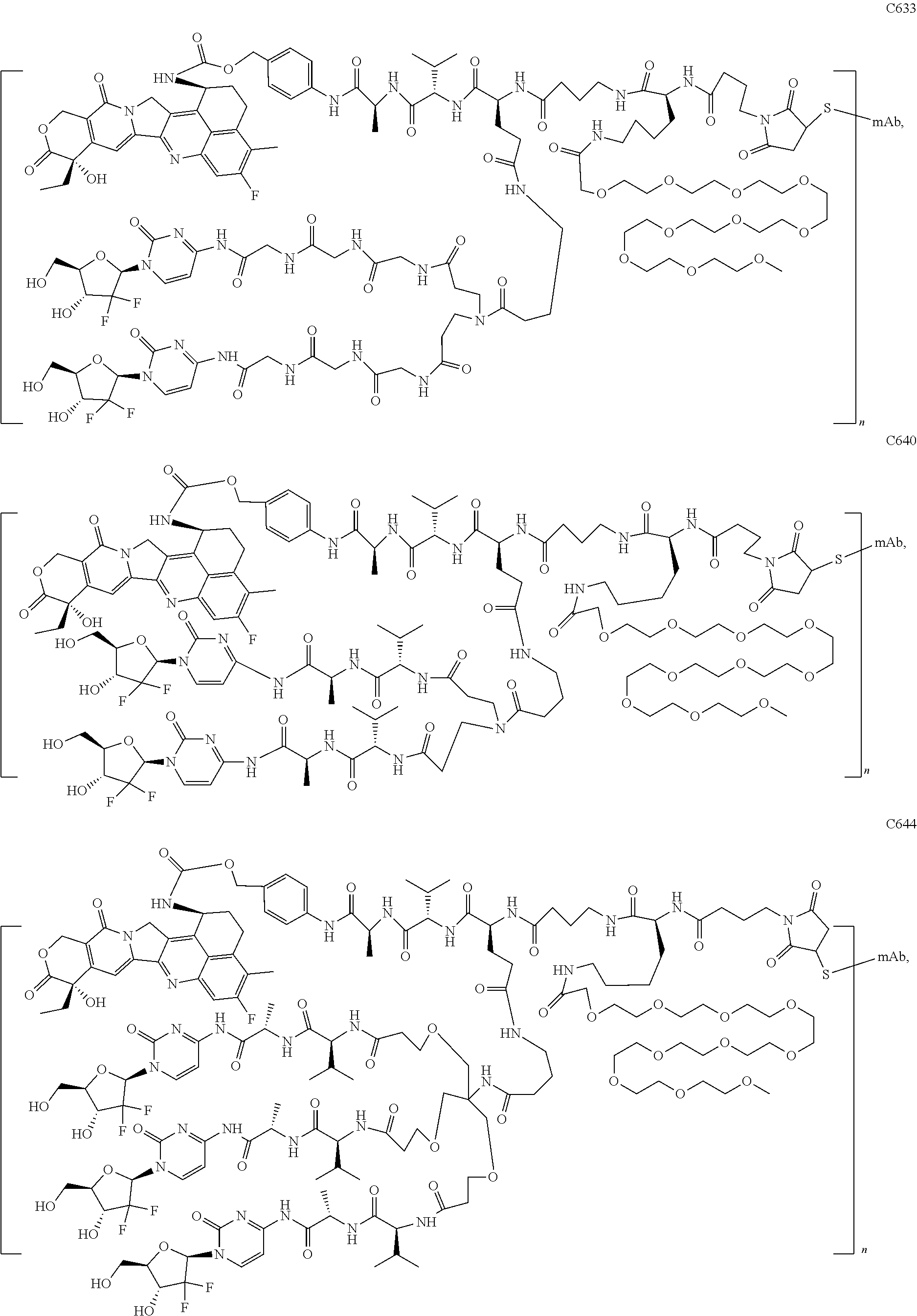

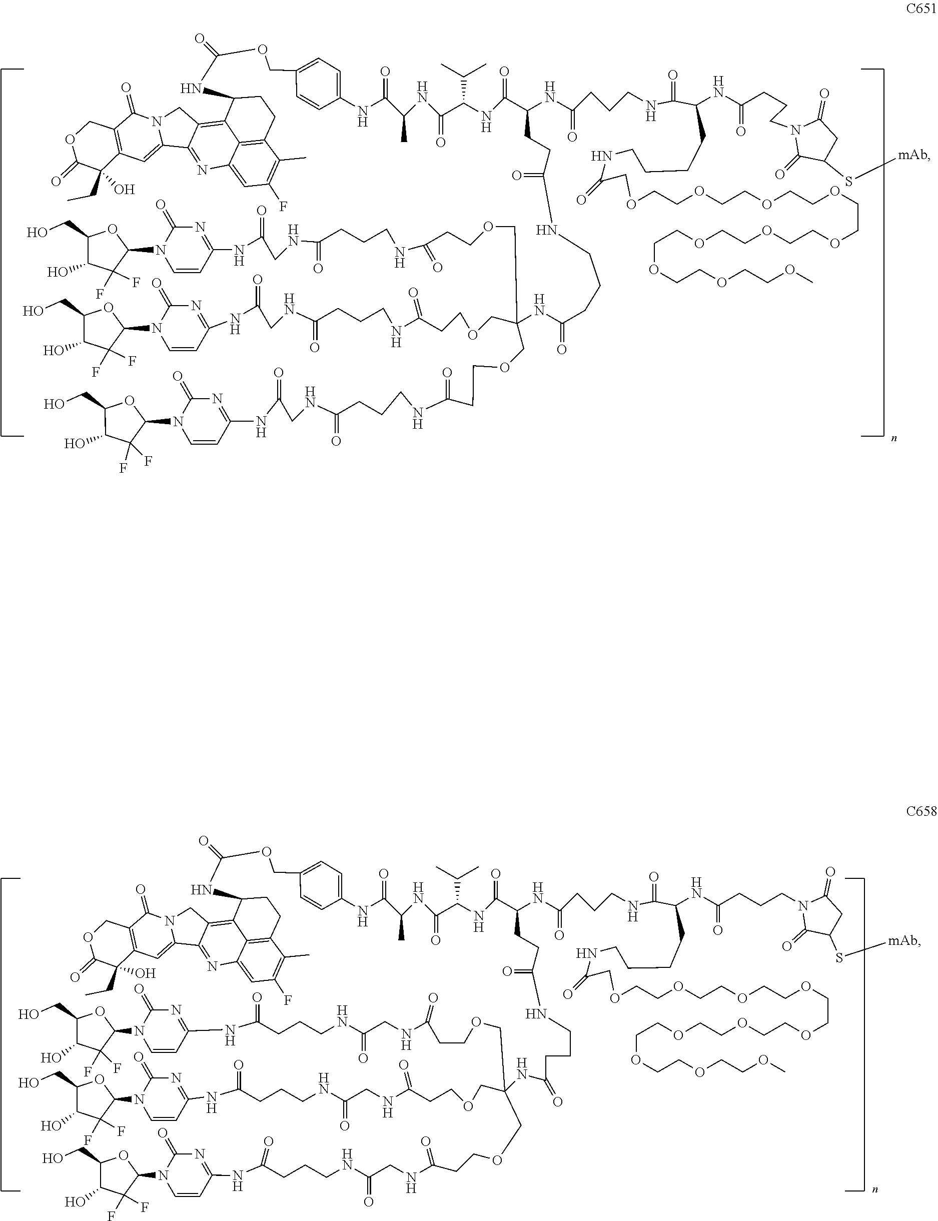

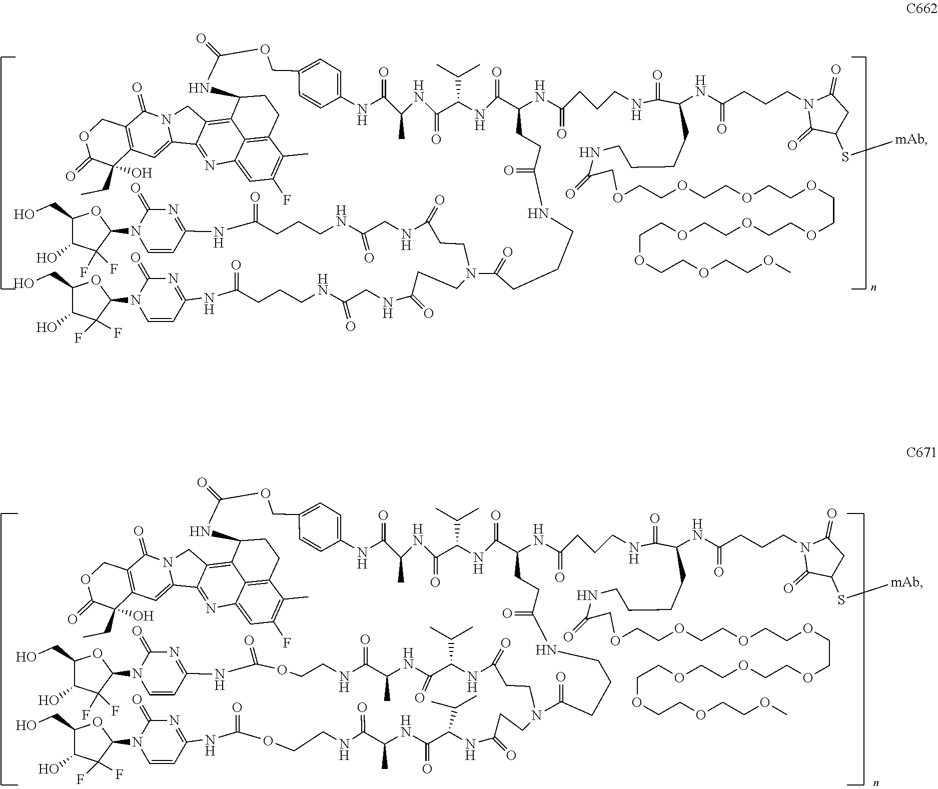

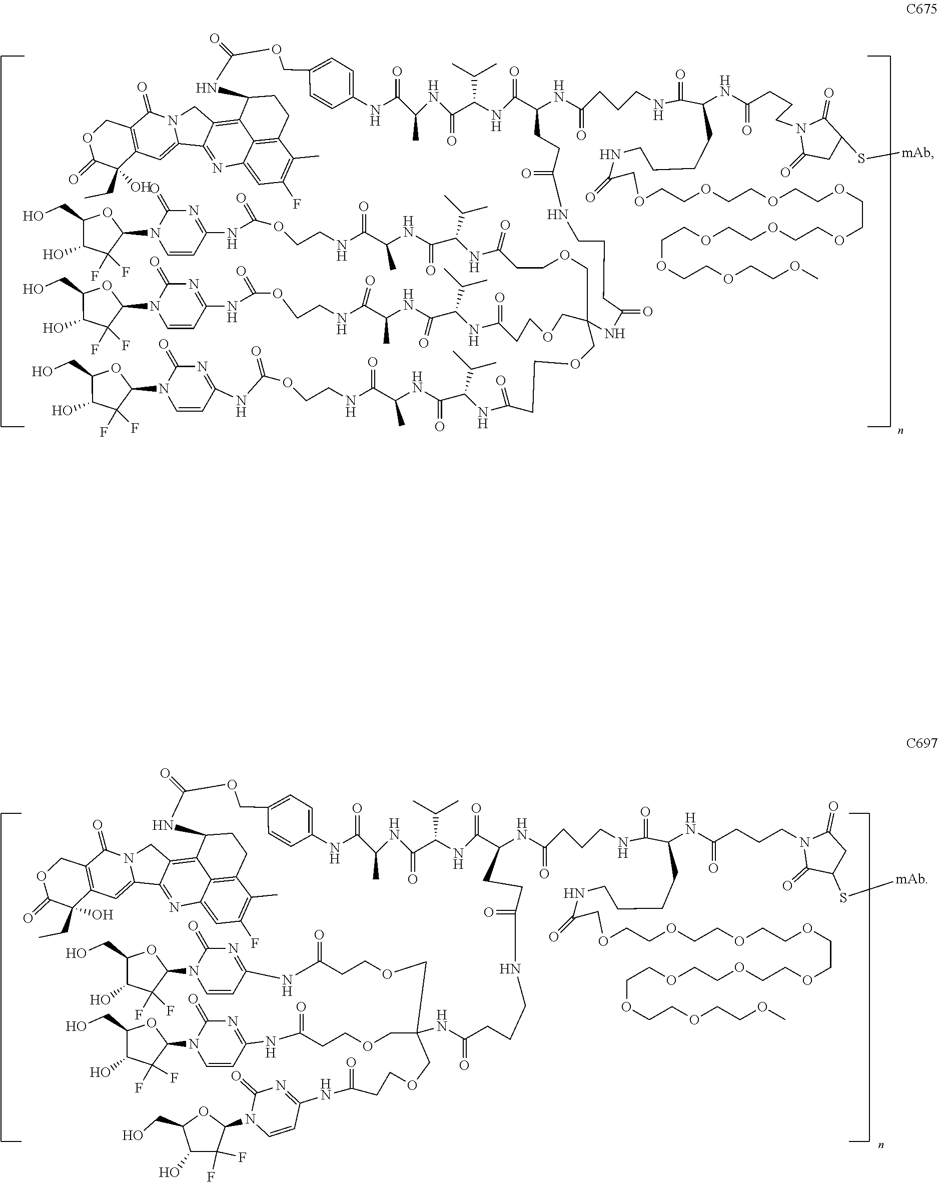

### Example 479. General preparation of formulation of the conjugates

In a liquid formulation of 80 mg of each conjugate: C025, C030, C043, C047, C052, C072, C077, C082, C095, C102, C107, C134, C141, C150, C156, C167, C169, C176, C190, C202, C204, C211, C213, C222, C248, C254, C269, C274, C284, C289, C293, C297, C314, C324, C339, C342, C358, C361, C366, C376, C380, C383, C386, C390, C393, C396, C400, C403, C416, C418, C423, C427, C433, C439, C455, C467, C534, C536, C540, C541, C542, C571 (C571a, C571b, C571c, C571d, C571e, C571f, C571g, C571h, C571i, C571j, C571k, C5711, C571m, C571n, C571o, C571p, C571q, C571r), C572 (C572a, C572b, C572c, C572d, C572e, C572f, C572g, C572h, C572i, C572j, C572k, C5721, C572m, C572n, C572o, C572p, C572q), C573 (C573a, C573b, C573c, C573d, C573e, C573f, C573g, C573h, C573i, C573j, C573k, C5731, C573m, C573n, C573o, C573p, C573q), C574 (C574a, C574b, C574c, C574d, C574e, C574f, C574g, C574h, C574i, C574j, C574k, C5741, C574m, C574n), C575 (C575a, C575b, C575c, C575d, C575e, C575f, C575g, C575h, C575i, C575j, C575k, C5751, C575m), C576 (C576a, C576b, C576c, C576d, C576e, C576f, C576g, C576h, C576i, C576j, C576k, C5761, C576m, C576n), C577 (C577a, C577b, C577c, C577d, C577e, C577f, C577g, C577h, C577i, C577j, C577k, C5771, C577m, C577n), C578 (C578a, C578b, C578c, C578d, C578e, C578f, C578g, C578h, C578i, C578j, C578k, C5781, C578m, C578n), C579 (C579a, C579b, C579c, C579d, C579e, C579f, C579g, C579h, C579i, C579j, C579k, C5791, C579m), C580 (C580a, C580b, C580c, C580d, C580e, C580f, C580g, C580h, C580i, C580j, C580k, C5801, and C580m), C612, C613, C619, C621, C629, C633, C640, C644, C651, C658, C662, C671, C675 and C679 in the 10 mL of borosilicate vial containing 240 mg of sucrose, 0.8 mg of polysorbate-80, 24 mg of sodium citrate in 4 mL of sterile water were adjusted with citric acid to pH 5.5. Then each of the conjugate solution was lyophilized at temperature from −65° C. to 0° C., and to RT at reduced pressure (5~10 torr) to form a dryness cake. The cake conjugates were stored at 2~8° C., and then reconstituted with 4 mL of sterile water for further application.

### Example 480. PC-3-4H7 cell line develop

Parent cell line PC-3 from Nanjing Cobioer Biosciences Co., was introduced with plasmid coexpress both human PSMA and RFP-Neomycin fusion protein and plasmid coexpress human Steap1 and GFP-Blasticidin fusion protein. The high expression cells were selected by neomycin and Blasticidin, and the selected clone 4H7 having high expression of both PSMA and Steapl was verified by FACs and chosen for further use in vitro and in vivo study.

Example 481 In vitro cytotoxicity of the Steapl, B7H₃, Trop2 and DLL3 ADCs at DAR=4.0+0.3 with payloads/linker complexes of this patent application having the structures of: C025, C030, C043, C047, C052, C072, C077, C082, C095, C102, C107, C297, C314, C324, C339, C342, C358, C361, C366, C376, C380, C383, C386, C390, C393, C396, C400, C403, C455, and C467, were evaluated in comparison with the Dxd-GGFG conjugates (DAR=4.0±0.4) of Steap1, B7H₃, Trop2 and DLL3 antibodies accordingly. Trop2, Folate, Her2, EGFR, DLL3, cMet, Muel, Her2 bispecific (trastuzumab/pertuzumab, BsHer2), Her3 and B7H3 antibodies were conjugated at DAR=4.0±0.4 selectively with payloads/linker complexes of this patent application to have the structures of C134, C141, C150, C156, C167, C169, C176, C190, C202, C204, C211, C213, C222, C248, C254, C269, C274, C284, C289, C293, C297, C314, C324, C339, C342, C416, C418, C423, C427, C433, C439, C455, C467, C534, C536, C540, C541, C542, C571 (C571a, C571b, C571c, C571d, C571e, C571f, C571g, C571h, C571i, C571j, C571k, C5711, C571m, C571n, C571o, C571p, C571q, C571r), C572 (C572a, C572b, C572c, C572d, C572e, C572f, C572g, C572h, C572i, C572j, C572k, C5721, C572m, C572n, C572o, C572p, C572q), C573 (C573a, C573b, C573c, C573d, C573e, C573f, C573g, C573h, C573i, C573j, C573k, C5731, C573m, C573n, C573o, C573p, C573q), C574 (C574a, C574b, C574c, C574d, C574e, C574f, C574g, C574h, C574i, C574j, C574k, C5741, C574m, C574n), C575 (C575a, C575b, C575c, C575d, C575e, C575f, C575g, C575h, C575i, C575j, C575k, C5751, C575m), C576 (C576a, C576b, C576c, C576d, C576e, C576f, C576g, C576h, C576i, C576j, C576k, C5761, C576m, C576n), C577 (C577a, C577b, C577c, C577d, C577e, C577f, C577g, C577h, C577i, C577j, C577k, C5771, C577m, C577n), C578 (C578a, C578b, C578c, C578d, C578e, C578f, C578g, C578h, C578i, C578j, C578k, C5781, C578m, C578n), C579 (C579a, C579b, C579c, C579d, C579e, C579f, C579g, C579h, C579i, C579j, C579k, C5791, C579m), C580 (C580a, C580b, C580c, C580d, C580e, C580f, C580g, C580h, C580i, C580j, C580k, C5801, C580m), C612, C613, C619, C621, C629, C633, C640, C644, C651, C658, C662, C671, C675 and C679. Their in vitro cytotoxicities were also evaluated in comparison with the Dxd-GGFG conjugates (DAR=4.0±0.4) of the same antibodies accordingly and along with Paclitaxel as a control. Since the nucleoside antimetabolites/analogs and the binding ligands are 50 to over 1000 times of less potent than the cytotoxic CPT compounds or Tubulysin analogs or eribulin compounds in the application, thus the DAR is only counted the potent cytotoxic compounds in the ADC conjugates when the other small molecules of this patent are conjugated in the conjugates.

The cell lines used in the cytotoxicity assays were: C4-2B Cells which was obtained from ATCC through license agreement with MD Anderson Cancer Center, Houston, TX, USA. 22RV1, U87, NCI-N87, MKN-7, JIMT-1, SU86.86, SW1990, OVCAR-3, Calu-3, A431, AsPC-1, HCC827 and H1975 cells were purchased from ATCC, Nanjing Cobioer and The Cell Bank of Shanghai Institute of Biochemistry and Cell Biology, and the others. C4-2B and PC-3-4H7 are both Steap1 and PSMA antigen high express cells, but medium expressed of both B7H3 and DLL3 antigens. 22RV1 is low express cells for Steapl, PSMA, B7H3 and DLL3 antigens. U87 is glioblastoma (GBM) cells, NCI-N87 and MKN-7 cell line are stable models of the gastric epithelium. A431 is human epidermoid carcinoma. JIM-1 is trastuzumab-resistant Her2 breast tumor cells. SU86.86, SW1990 and AsPC-1 are pancreatic cancer cell lines. OVCAR-3 is ovarian cancer cell lines. Calu-3, HCC827 and NCI-H1975 are a human lung adenocarcinoma. All these cells were grown according to the provider manuals. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/mL) was then added to the wells (20 μl) and the plates were incubated for 1.5 hrs. at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition % =[1-(assay-blank)/(control-blank)]x 100. Some of the in vitro activities (MTT results) are listed in Table 2 and Table 3 below.

TABLE 2
MTT assays of the B7H3-ADCs against tumor cells of C4-
2B, PC3-4H7 and 22RV1, at 6000 cells, 96 h incubation:

| ADC | DAR | IC50 (nM) | IC50 (nM) | IC50 (nM) |
|---|---|---|---|---|
| Compound | ratio | C4-2B | PC3-4H7 | 22RV1 |
| C025 | 4.1 | 0.042 | 0.039 | 10.53 |
| C030 | 4.1 | 0.051 | 0.058 | 9.77 |
| C043 | 4.1 | 0.047 | 0.053 | 10.26 |
| C047 | 4.2 | 0.054 | 0.048 | 10.13 |
| C052 | 4.0 | 0.057 | 0.049 | 11.06 |
| C072 | 4.1 | 0.046 | 0.059 | 10.66 |
| C077 | 4.1 | 0.055 | 0.061 | 11.37 |
| C082 | 4.2 | 0.087 | 0.071 | 12.35 |
| C095 | 4.1 | 0.028 | 0.021 | 5.93 |
| C102 | 4.2 | 0.022 | 0.030 | 5.85 |
| C107 | 4.1 | 0.024 | 0.029 | 4.92 |
| C297 | 4.3 | 1.98 | 1.78 | 23.35 |
| C314 | 4.3 | 0.23 | 0.18 | 11.78 |
| C324 | 4.3 | 0.55 | 0.61 | 65.9 |
| C339 | 4.3 | 2.81 | 2.18 | 72.45 |
| C342 | 4.3 | 2.13 | 2.69 | 79.70 |
| C358 | 4.3 | 0.35 | 0.68 | 41.85 |
| C361 | 4.2 | 0.18 | 0.37 | 22.32 |
| C366 | 4.2 | 0.22 | 0.27 | 39.78 |
| C376 | 4.2 | 0.65 | 0.58 | 42.32 |
| C380 | 4.0 | 0.86 | 0.69 | 53.58 |
| C383 | 4.1 | 0.26 | 0.35 | 31.23 |
| C386 | 4.2 | 2.79 | 2.62 | 71.85 |
| C390 | 4.2 | 0.89 | 0.77 | 28.2 |
| C393 | 4.2 | 3.72 | 3.85 | 33.85 |
| C396 | 4.2 | 0.85 | 1.12 | 21.57 |
| C400 | 4.2 | 0.97 | 0.81 | 41.3 |
| C403 | 4.2 | 0.75 | 0.63 | 20.31 |
| C455 | 4.2 | 2.87 | 2.43 | 42.64 |
| C467 | 4.2 | 2.03 | 1.95 | 39.68 |
| B7H3- | 4.2 | 8.57 | 7.82 | 123.57 |
| GGFG-Dxd | | | | |
| Paclitaxel | — | 1.59 | 2.03 | 1.85 |

TABLE 3
MTT assays of the BsHer2 (trastuzumab/pertuzumab)-ADCs
against Her2 positive tumor cells of NCI-N87, MKN-7,
SW1990 and SU86.86, at 5000 cells, 96 h incubation:

| ADC | DAR | IC50 (nM) | IC50 (nM) | IC50 (nM) | IC50 (nM) |
|---|---|---|---|---|---|
| Compound | ratio | NCI-N87 | MKN-7 | SW1990 | SU86.86 |
| C134 | 4.1 | 0.023 | 78.75 | 0.82 | 1.53 |
| C141 | 4.1 | 0.42 | 102.84 | 0.78 | 6.71 |
| C150 | 4.2 | 1.28 | 43.52 | 0.53 | 10.32 |
| C156 | 4.2 | 1.07 | 55.53 | 0.87 | 9.13 |
| C167 | 4.0 | 2.06 | 61.72 | 0.59 | 11.38 |
| C169 | 4.1 | 1.82 | 53.41 | 0.49 | 9.68 |
| C176 | 4.0 | 0.019 | 23.55 | 0.31 | 1.30 |
| C190 | 4.2 | 0.81 | 31.87 | 0.71 | 2.25 |
| C202 | 4.0 | 1.83 | 40.38 | 0.82 | 7.93 |
| C204 | 4.2 | 1.62 | 35.27 | 0.93 | 9.81 |
| C211 | 4.1 | 0.092 | 20.28 | 0.52 | 4.90 |
| C213 | 4.2 | 1.18 | 31.83 | 0.73 | 5.33 |
| C222 | 4.1 | 2.07 | 40.22 | 0.68 | 6.08 |
| C248 | 4.2 | 2.01 | 56.52 | 0.81 | 5.93 |
| C254 | 4.1 | 1.95 | 57.83 | 0.88 | 7.43 |
| C269 | 4.2 | 2.12 | 58.63 | 0.72 | 9.15 |
| C274 | 4.2 | 1.98 | 49.85 | 0.69 | 9.52 |
| C284 | 4.0 | 2.00 | 50.58 | 0.67 | 10.31 |
| C289 | 4.2 | 0.019 | 8.29 | 0.17 | 3.08 |
| C297 | 4.2 | 2.67 | 207.65 | 20.89 | 82.28 |
| C314 | 4.0 | 0.091 | 20.06 | 0.92 | 6.55 |
| C324 | 4.0 | 0.082 | 10.26 | 0.39 | 3.27 |
| C339 | 4.2 | 3.21 | 82.79 | 12.62 | 78.05 |
| C342 | 4.2 | 1.18 | 30.89 | 1.77 | 8.22 |
| C416 | 4.2 | 0.091 | 5.72 | 1.05 | 3.15 |
| C418 | 4.2 | 1.82 | 58.89 | 1.42 | 11.53 |
| C423 | 4.2 | 1.87 | 60.92 | 1.81 | 12.39 |
| C427 | 4.2 | 0.12 | 20.75 | 0.83 | 6.35 |
| C433 | 4.2 | 0.10 | 15.80 | 1.41 | 7.68 |
| C439 | 4.1 | 0.13 | 22.13 | 1.55 | 7.85 |
| C455 | 4.2 | 2.42 | 68.86 | 10.53 | 22.72 |
| C467 | 4.2 | 2.17 | 61.59 | 11.82 | 23.55 |
| C534 | 4.2 | 0.086 | 20.12 | 0.83 | 4.77 |
| C536 | 4.1 | 0.21 | 21.17 | 1.12 | 5.45 |
| C540 | 4.2 | 0.46 | 26.43 | 1.47 | 6.76 |
| C541 | 4.1 | 0.21 | 23.82 | 1.53 | 5.73 |
| C542 | 4.1 | 1.28 | 28.17 | 1.72 | 6.80 |
| C571a | 4.2 | 1.42 | 36.67 | 5.28 | 14.36 |
| C571b | 4.2 | 1.18 | 32.83 | 5.11 | 13.82 |
| C571c | 4.1 | 1.24 | 33.52 | 5.09 | 14.12 |
| C571d | 4.1 | 0.78 | 28.10 | 5.17 | 13.24 |
| C571e | 4.0 | 2.33 | 52.52 | 6.14 | 18.78 |
| C571f | 4.0 | 2.46 | 55.68 | 6.06 | 19.37 |
| C571g | 4.1 | 2.48 | 54.39 | 6.23 | 19.51 |
| C571h | 4.2 | 2.10 | 50.23 | 5.99 | 18.42 |
| C571i | 4.2 | 2.22 | 51.14 | 6.08 | 19.13 |
| C571j | 4.2 | 1.82 | 58.12 | 5.64 | 16.82 |
| C571k | 3.9 | 2.50 | 56.82 | 6.48 | 19.83 |
| C571l | 4.2 | 2.30 | 55.66 | 6.25 | 19.37 |
| C571m | 4.2 | 2.41 | 57.93 | 6.68 | 20.26 |
| C571n | 4.2 | 2.46 | 65.90 | 6.97 | 21.87 |
| C571o | 4.2 | 2.58 | 66.43 | 6.87 | 22.05 |
| C571p | 4.2 | 2.03 | 64.22 | 6.77 | 22.14 |
| C571q | 4.2 | 2.61 | 67.74 | 7.11 | 23.23 |
| C571r | 4.2 | 1.44 | 27.12 | 6.04 | 16.62 |
| C572a | 4.1 | 0.82 | 18.89 | 3.61 | 10.05 |
| C572b | 4.1 | 0.68 | 12.35 | 2.87 | 8.80 |
| C572c | 4.0 | 0.74 | 13.08 | 3.17 | 9.18 |
| C572d | 4.0 | 0.66 | 8.17 | 1.97 | 7.28 |
| C572e | 4.0 | 1.23 | 24.31 | 2.84 | 9.89 |
| C572f | 4.1 | 1.04 | 21.12 | 1.76 | 9.07 |
| C572g | 4.1 | 1.07 | 20.33 | 1.63 | 8.51 |
| C572h | 4.2 | 1.10 | 20.03 | 1.59 | 8.41 |
| C572i | 4.2 | 0.82 | 8.04 | 1.08 | 8.13 |
| C572j | 4.0 | 0.95 | 21.19 | 1.44 | 9.80 |
| C572k | 4.1 | 1.82 | 20.83 | 2.48 | 12.03 |
| C572l | 4.2 | 2.02 | 22.06 | 2.97 | 11.09 |
| C572m | 4.2 | 2.61 | 51.93 | 5.68 | 16.06 |
| C572n | 4.2 | 2.68 | 53.96 | 5.97 | 15.83 |
| C572o | 4.2 | 2.43 | 51.43 | 5.75 | 15.05 |
| C572p | 4.2 | 2.33 | 54.22 | 5.17 | 16.17 |
| C572q | 4.2 | 1.01 | 11.74 | 1.71 | 6.23 |
| C573a | 4.3 | 1.92 | 39.78 | 5.79 | 18.77 |
| C573b | 4.2 | 1.48 | 35.64 | 5.65 | 16.63 |
| C573c | 4.2 | 1.67 | 36.81 | 5.70 | 17.26 |
| C573d | 4.4 | 1.05 | 29.88 | 5.72 | 14.53 |
| C573e | 4.0 | 2.63 | 54.78 | 6.69 | 20.84 |
| C573f | 4.0 | 2.56 | 55.97 | 6.79 | 21.92 |
| C573g | 4.3 | 2.40 | 53.52 | 6.08 | 19.78 |
| C573h | 4.2 | 2.66 | 54.86 | 6.65 | 20.48 |
| C573i | 4.3 | 2.01 | 50.43 | 5.84 | 17.37 |
| C573j | 4.2 | 2.63 | 58.97 | 5.99 | 18.91 |
| C573k | 4.3 | 2.51 | 56.23 | 6.82 | 18.95 |
| C573l | 4.2 | 2.60 | 57.85 | 6.95 | 19.97 |
| C573m | 4.2 | 2.43 | 63.98 | 6.87 | 24.05 |
| C573n | 4.2 | 2.59 | 69.83 | 7.54 | 25.52 |
| C573o | 4.3 | 2.47 | 68.74 | 7.32 | 23.11 |
| C573p | 4.2 | 2.52 | 70.26 | 7.80 | 25.36 |
| C573q | 4.3 | 2.09 | 55.43 | 6.32 | 17.52 |
| C574a | 4.1 | 0.84 | 18.96 | 3.53 | 9.87 |
| C574b | 4.2 | 0.71 | 12.77 | 2.98 | 8.94 |
| C574c | 4.0 | 0.74 | 12.92 | 2.86 | 9.03 |
| C574d | 4.3 | 0.67 | 8.97 | 1.90 | 8.42 |
| C574e | 4.1 | 1.17 | 22.31 | 2.88 | 9.93 |
| C574f | 4.1 | 1.20 | 21.49 | 2.01 | 9.23 |
| C574g | 4.2 | 1.03 | 20.02 | 1.74 | 8.82 |
| C574h | 4.2 | 1.07 | 19.88 | 1.63 | 8.27 |
| C574i | 4.2 | 0.73 | 7.89 | 1.02 | 8.07 |
| C574j | 4.1 | 1.04 | 21.79 | 1.40 | 9.93 |
| C574k | 4.2 | 1.95 | 21.41 | 2.67 | 12.86 |
| C574l | 4.2 | 2.23 | 22.76 | 2.97 | 12.39 |
| C574m | 4.2 | 2.42 | 50.77 | 5.34 | 21.78 |
| C574n | 4.1 | 2.67 | 53.72 | 5.37 | 22.99 |
| C574o | 4.2 | 1.11 | 12.03 | 2.08 | 7.73 |
| C575a | 4.2 | 1.83 | 38.85 | 5.47 | 17.91 |
| C575b | 4.2 | 1.56 | 35.33 | 5.42 | 16.73 |
| C575c | 4.3 | 1.57 | 36.02 | 5.40 | 16.39 |
| C575d | 4.3 | 1.16 | 29.70 | 5.63 | 15.22 |
| C575e | 4.2 | 2.63 | 55.85 | 6.92 | 21.66 |
| C575f | 4.1 | 2.61 | 56.34 | 6.97 | 22.26 |
| C575g | 4.2 | 2.26 | 51.87 | 6.02 | 18.55 |
| C575h | 4.1 | 2.70 | 64.96 | 7.82 | 24.98 |
| C575i | 4.2 | 2.67 | 69.90 | 7.38 | 26.84 |
| C575j | 4.2 | 2.68 | 67.47 | 7.54 | 25.88 |
| C575k | 4.1 | 2.63 | 69.64 | 8.32 | 27.98 |
| C575l | 4.3 | 2.09 | 55.43 | 6.32 | 17.52 |
| C576a | 4.1 | 0.134 | 10.96 | 3.53 | 5.87 |
| C576b | 4.2 | 0.121 | 10.07 | 2.38 | 4.94 |
| C576c | 4.0 | 0.142 | 10.72 | 2.86 | 5.03 |
| C576d | 4.2 | 0.077 | 3.97 | 1.23 | 2.42 |
| C576e | 4.1 | 0.158 | 6.33 | 2.88 | 3.93 |
| C576f | 4.2 | 0.122 | 6.09 | 2.01 | 4.41 |
| C576g | 4.1 | 0.232 | 6.21 | 1.30 | 4.12 |
| C576h | 4.2 | 0.129 | 5.63 | 1.02 | 4.07 |
| C576i | 4.1 | 0.313 | 6.89 | 1.42 | 4.57 |
| C576j | 4.2 | 0.424 | 6.79 | 1.57 | 4.89 |
| C576k | 4.2 | 0.491 | 6.93 | 1.63 | 4.66 |
| C576l | 4.2 | 0.387 | 6.76 | 2.07 | 4.49 |
| C576m | 4.2 | 0.428 | 6.87 | 2.34 | 4.73 |
| C576n | 4.2 | 0.106 | 4.79 | 2.17 | 4.98 |
| C577a | 4.2 | 1.53 | 33.77 | 5.21 | 14.55 |
| C577b | 4.2 | 1.43 | 30.33 | 4.62 | 13.63 |
| C577c | 4.2 | 1.57 | 36.02 | 5.03 | 14.39 |
| C577d | 4.3 | 1.76 | 37.70 | 5.43 | 13.82 |
| C577e | 4.2 | 1.89 | 39.89 | 5.94 | 19.66 |
| C577f | 4.1 | 2.21 | 39.94 | 6.37 | 19.26 |
| C577g | 4.2 | 2.03 | 34.87 | 5.92 | 17.55 |
| C577h | 4.0 | 1.91 | 32.97 | 5.82 | 15.93 |
| C577i | 4.2 | 1.39 | 29.90 | 4.88 | 16.01 |
| C577j | 4.2 | 2.49 | 39.96 | 5.59 | 19.83 |
| C577k | 4.3 | 2.55 | 48.63 | 6.37 | 21.57 |
| C577l | 4.2 | 2.21 | 50.07 | 6.43 | 20.02 |
| C577m | 4.1 | 2.53 | 64.71 | 7.13 | 25.86 |
| C577n | 4.3 | 1.29 | 35.40 | 4.53 | 12.26 |
| C578a | 4.2 | 0.088 | 5.78 | 2.29 | 3.19 |
| C578b | 4.2 | 0.079 | 5.54 | 2.16 | 2.98 |
| C578c | 4.2 | 0.087 | 5.58 | 2.19 | 3.07 |
| C578d | 4.0 | 0.066 | 3.73 | 1.17 | 2.28 |
| C578e | 4.1 | 0.120 | 5.81 | 2.57 | 3.42 |
| C578f | 4.2 | 0.112 | 5.09 | 2.18 | 3.51 |
| C578g | 4.1 | 0.102 | 4.87 | 1.99 | 3.31 |
| C578h | 4.2 | 0.109 | 4.13 | 2.08 | 2.95 |
| C578i | 4.0 | 0.113 | 4.61 | 2.42 | 3.57 |
| C578j | 4.2 | 0.144 | 5.39 | 2.63 | 2.98 |
| C578k | 4.2 | 0.191 | 5.61 | 2.69 | 3.29 |
| C578l | 4.2 | 0.227 | 5.76 | 2.74 | 3.49 |
| C578m | 4.3 | 0.251 | 5.87 | 2.93 | 3.77 |
| C578n | 4.2 | 0.086 | 3.99 | 2.11 | 2.38 |
| C579a | 4.3 | 0.129 | 7.87 | 4.73 | 6.16 |
| C579b | 4.2 | 0.120 | 7.12 | 4.47 | 5.16 |
| C579c | 4.0 | 0.125 | 7.43 | 4.63 | 5.67 |
| C579d | 4.2 | 0.073 | 4.12 | 1.78 | 2.39 |
| C579e | 4.2 | 0.150 | 6.01 | 2.86 | 3.78 |
| C579f | 4.2 | 0.127 | 6.05 | 2.48 | 3.71 |
| C579g | 4.1 | 0.121 | 5.71 | 2.09 | 3.37 |
| C579h | 4.0 | 0.148 | 5.82 | 2.01 | 3.07 |
| C579i | 4.2 | 0.170 | 6.12 | 1.85 | 3.98 |
| C579j | 4.2 | 0.192 | 6.57 | 1.97 | 4.22 |
| C579k | 4.2 | 0.289 | 6.43 | 1.87 | 4.52 |
| C579l | 4.2 | 0.303 | 6.78 | 2.06 | 4.67 |
| C579m | 4.2 | 0.083 | 4.61 | 1.82 | 2.36 |
| C580a | 4.3 | 0.111 | 3.80 | 3.92 | 3.55 |
| C580b | 4.2 | 0.103 | 3.38 | 3.61 | 4.04 |
| C580c | 4.0 | 0.117 | 3.67 | 3.96 | 4.77 |
| C580d | 4.2 | 0.068 | 2.37 | 1.72 | 2.21 |
| C580e | 4.2 | 0.117 | 5.07 | 1.89 | 2.89 |
| C580f | 4.2 | 0.109 | 4.87 | 1.67 | 2.78 |
| C580g | 4.1 | 0.089 | 3.71 | 1.93 | 2.98 |
| C580h | 4.0 | 0.128 | 4.69 | 2.22 | 3.13 |
| C580i | 4.2 | 0.151 | 5.72 | 1.79 | 2.96 |
| C580j | 4.2 | 0.172 | 6.21 | 1.99 | 3.29 |
| C580k | 4.2 | 0.223 | 6.44 | 2.35 | 4.22 |
| C580l | 4.2 | 0.273 | 6.95 | 3.11 | 4.54 |
| C580m | 4.1 | 0.071 | 3.15 | 1.66 | 2.07 |
| C612 | 4.3 | 0.087 | 12.87 | 1.41 | 6.17 |
| C613 | 4.2 | 0.062 | 6.63 | 1.32 | 5.19 |
| C619 | 4.1 | 0.074 | 7.13 | 1.42 | 5.57 |
| C621 | 4.3 | 0.071 | 7.86 | 1.63 | 5.82 |
| C629 | 4.1 | 0.041 | 5.05 | 1.23 | 4.72 |
| C633 | 4.1 | 0.054 | 5.67 | 1.49 | 5.54 |
| C640 | 4.0 | 0.060 | 5.85 | 1.56 | 5.87 |
| C644 | 4.1 | 0.056 | 5.73 | 1.53 | 5.85 |
| C651 | 4.0 | 0.047 | 4.96 | 1.39 | 5.43 |
| C658 | 4.1 | 0.061 | 5.93 | 1.52 | 5.82 |
| C662 | 4.2 | 0.068 | 6.54 | 1.61 | 6.58 |
| C671 | 4.1 | 0.069 | 6.78 | 1.69 | 7.21 |
| C675 | 4.1 | 0.062 | 6.14 | 1.43 | 7.02 |
| C679 | 4.3 | 0.070 | 6.23 | 1.55 | 7.68 |
| Her2 | 4.2 | 2.72 | 353.2 | 48.78 | 241.8 |
| bispecific- | | | | | |
| GGFG-Dxd | | | | | |
| Paclitaxel | — | 0.76 | 10.18 | 0.042 | 2.98 |

Almost all ADCs tested above demonstrated better in vitro activities than GGFG-Dxd ADCs.

### Example 482. Antitumor Activities in vivo (BALB/c Nude Mice Bearing PC3-4H7 cells for Steapl-ADCs, B7H₃-ADCs and Trop2-ADCs; or bearing Calu 6 and HCC827 cells for B7H₃-ADC; or NCI-H292, A431, HCC827 or NCI-N87 for Trop2-ADCs; JIMT-1, SU86.86, or SW1990 cells for Her2 bispecific-ADCs or Muel-ADCs; or OVCAR-3, HCC827, A431 or H1975 cells for Folate-ADCs, Her3-ADC or EGFR-ADCs; Xenograft Tumors independently)

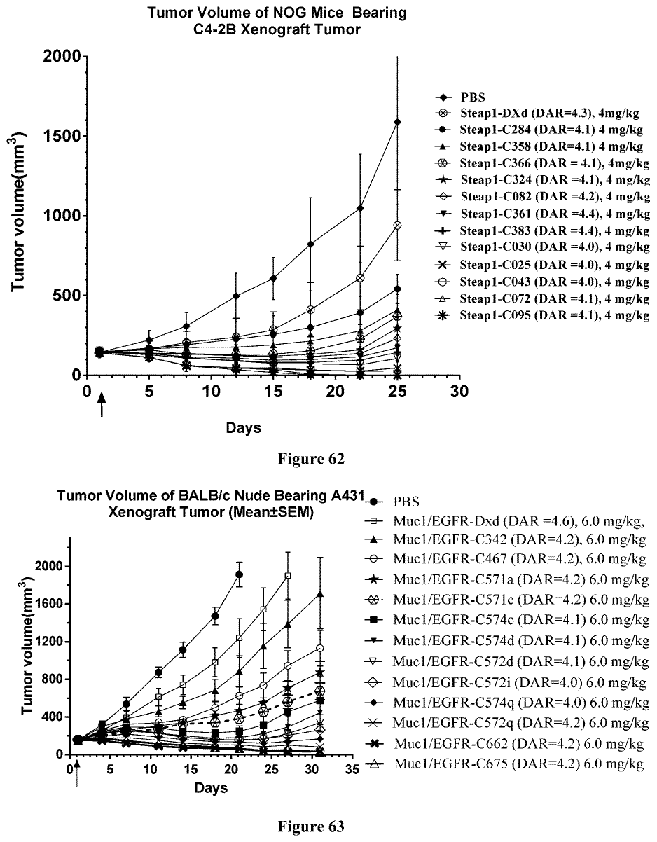

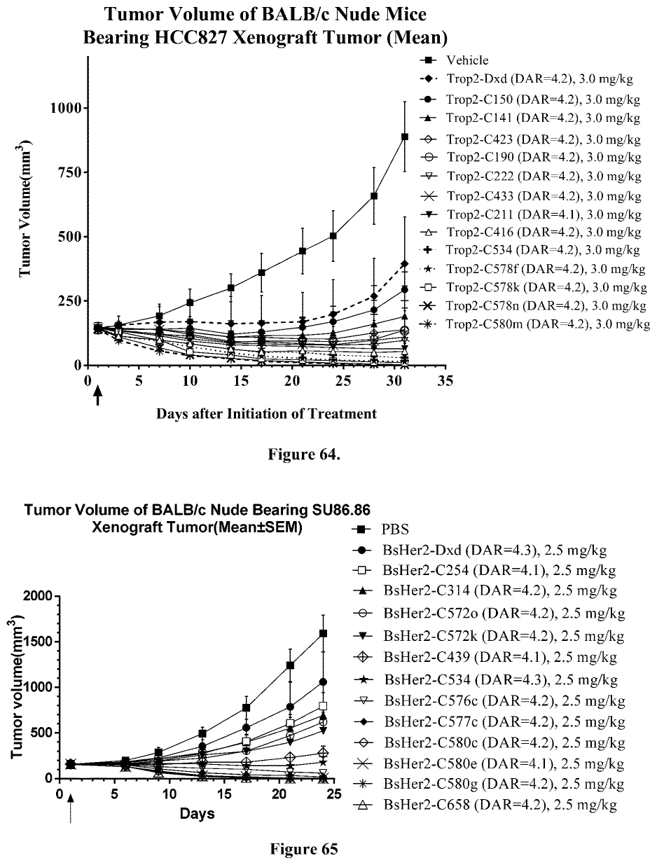

The in vivo efficacy of conjugates of C025, C030, C043, C047, C052, C072, C077, C082, C095, C102, C107, C134, C141, C150, C156, C167, C169, C176, C190, C202, C204, C211, C213, C222, C248, C254, C269, C274, C284, C289, C293, C297, C314, C324, C339, C342, C358, C361, C366, C376, C380, C383, C386, C390, C393, C396, C400, C403, C416, C418, C423, C427, C433, C439, C455, C467, C534, C536, C540, C541, C542, C571 (C571a, C571b, C571c, C571d, C571e, C571f, C571g, C571h, C571i, C571j, C571k, C5711, C571m, C571n, C571o, C571p, C571q, C571r), C572 (C572a, C572b, C572c, C572d, C572e, C572f, C572g, C572h, C572i, C572j, C572k, C5721, C572m, C572n, C572o, C572p, C572q), C573 (C573a, C573b, C573c, C573d, C573e, C573f, C573g, C573h, C573i, C573j, C573k, C5731, C573m, C573n, C573o, C573p, C573q), C574 (C574a, C574b, C574c, C574d, C574e, C574f, C574g, C574h, C574i, C574j, C574k, C5741, C574m, C574n), C575 (C575a, C575b, C575c, C575d, C575e, C575f, C575g, C575h, C575i, C575j, C575k, C5751, C575m), C576 (C576a, C576b, C576c, C576d, C576e, C576f, C576g, C576h, C576i, C576j, C576k, C5761, C576m, C576n), C577 (C577a, C577b, C577c, C577d, C577e, C577f, C577g, C577h, C577i, C577j, C577k, C5771, C577m, C577n), C578 (C578a, C578b, C578c, C578d, C578e, C578f, C578g, C578h, C578i, C578j, C578k, C5781, C578m, C578n), C579 (C579a, C579b, C579c, C579d, C579e, C579f, C579g, C579h, C579i, C579j, C579k, C5791, C579m), C580 (C580a, C580b, C580c, C580d, C580e, C580f, C580g, C580h, C580i, C580j, C580k, C5801, and C580m), C612, C613, C619, C621, C629, C633, C640, C644, C651, C658, C662, C671, C675 and C679 against tumor cells, in xenograft models were exampled in FIGS. 62-65. Five-week-old female BALB/c Nude mice (6 animals per group) were inoculated subcutaneously in the area under the right shoulder with respective carcinoma cells (5×10⁶ cells/mouse) in 0.1-0.2 mL of serum-free medium. The tumors were grown for 6-35 days to an average size of 150 mm³, or 8-40 days to an average size of 180 mm³. The animals were then randomly divided into different groups (6 animals per group). The first group of mice served as the control group and was treated with the phosphate-buffered saline (PBS) vehicle. The other groups were treated with conjugates at doses 1.0~8.0 mg/Kg (some of them were described in the figures), administered only once intravenously. If a control of paclitaxel was used, it was administrated at dose of 15 mg/Kg, once a week for three weeks intravenously. Three dimensions of the tumor were measured every 3 or 4 days (twice a week) and the tumor volumes were calculated using the formula tumor volume=1/2×(length ×width ×height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

The results of the examples were plotted in FIGS. 62-65. All the conjugates did not cause the animal body weight loss at the administrated doses or up to 300 mg/Kg. All conjugates demonstrated antitumor activity as comparison with PBS buffer, and most of the conjugates of invention showed better antitumor activities than the conjugates with the payload/linker complexes of GGFG-Dxd.

1. An antibody drug conjugate with a branch functional small molecule having the Formula (I), (II), (III) (IV) represented as: 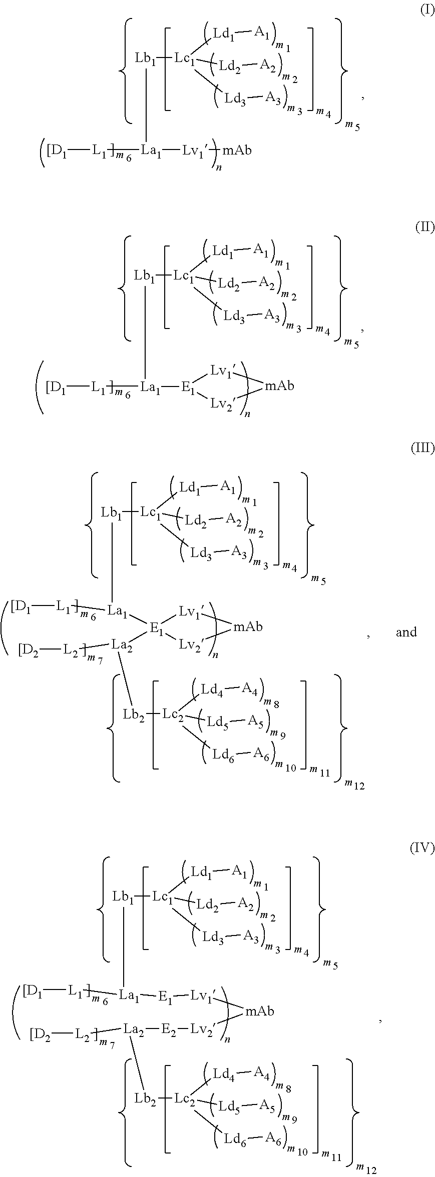
D₁ and D₂ are a cytotoxic agent; mAb is an antibody or antibody like protein; n is 1, 2, 3, 4, 5, 6, 7, 8, 9, 10, 11, 12, 13, 14, 15, 16, 17, 18, 19 or 20, and these numbers may have a decimal;
L₁, L₂, La₁, La₂, Lb₁, Lb₂, Lc₁, Lc₂, Ld₁, Ld₂, Ld₃, Ld₄, Ld₅, and Ld₆ are a linker, which are independently selected from O, NH, S, N, NH—NH, N—N, N(R₃), N(R₃)N(R₃′), C(═O)N, C(═O)NH, C(═O)N—N, C₁-C₈ of alkyl (alkylene); C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, urea, carbamate, carbonate, thiourea, thioether, thiourea, amide, aminoalkylcarboxyl, thioalkylcarboxyl or oxylalkylcarboxyl; or 1-8 natural or unnatural amino acids; or polyethyleneoxy unit of formula (OCH₂CH₂)ₚOR₃, or (OCH₂CH(CH₃))ₚOR₃, or NH(CH₂CH₂O)ₚR₃, or NH(CH₂CH(CH₃)O)ₚR₃, or N[(CH₂CH₂O)ₚR₃][(CH₂CH₂O)ₚ,R₃′], or (OCH₂CH₂)ₚCOOR₃, or CH2CH2(OCH₂CH₂)ₚCOOR₃, wherein p and p′ are independently an integer selected from 0 to about 100, or combination thereof; wherein R₃ and R₃, are independently H, C(═O)H, C(═O)CH₃, C₁-C₈ of alkyl; or combination above thereof;
wherein Lv₁′ and Lv₂′ are independently or jointly having the following structures: 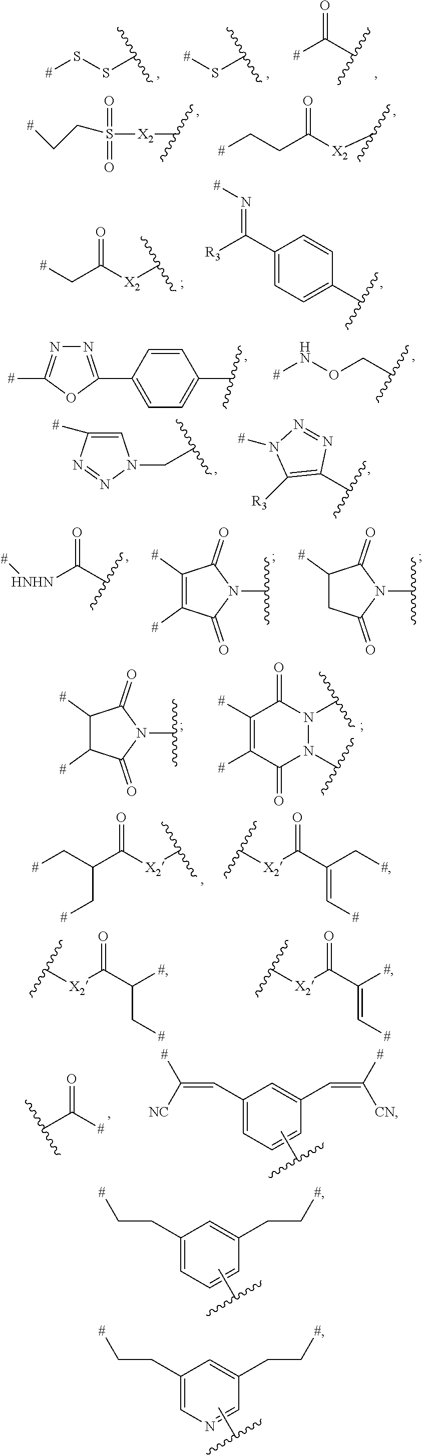 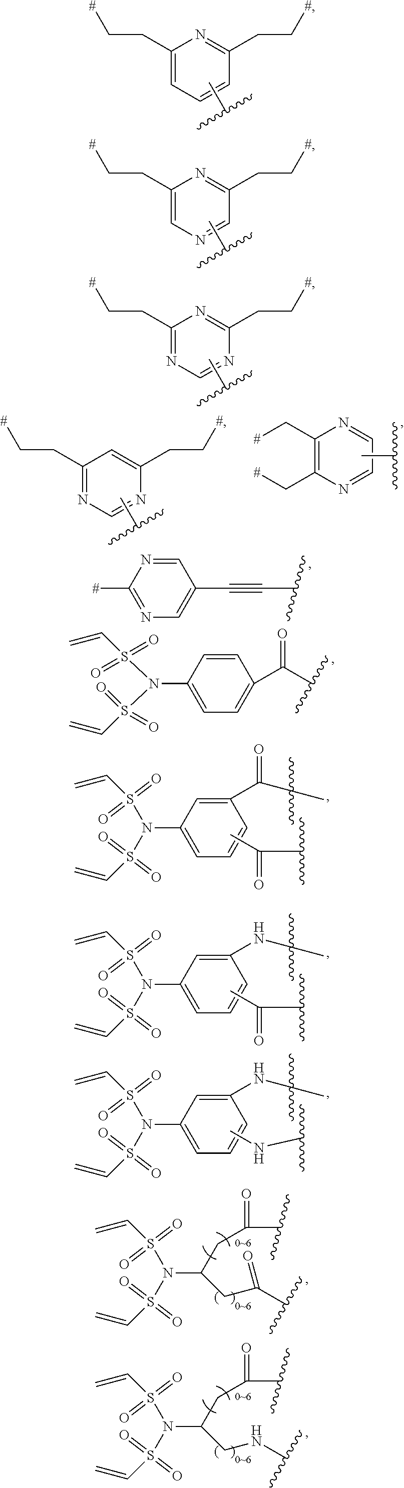 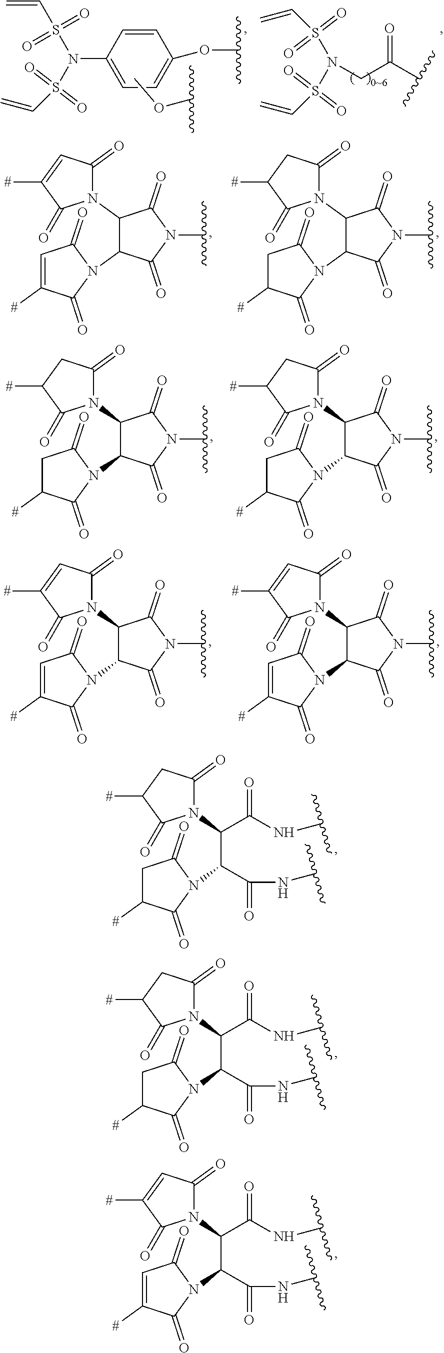 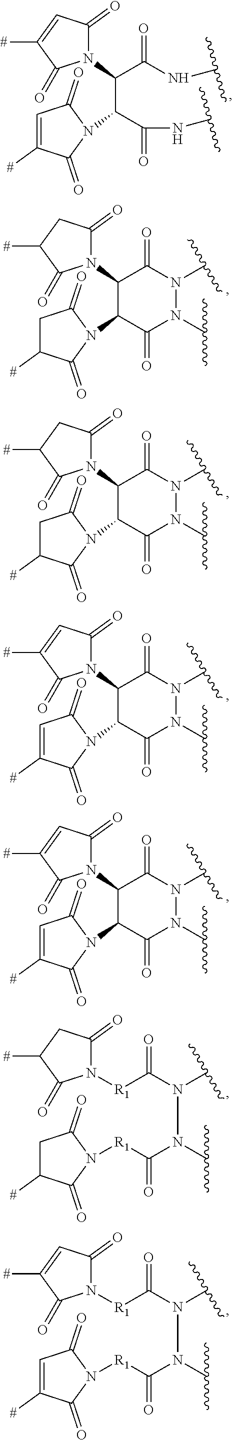 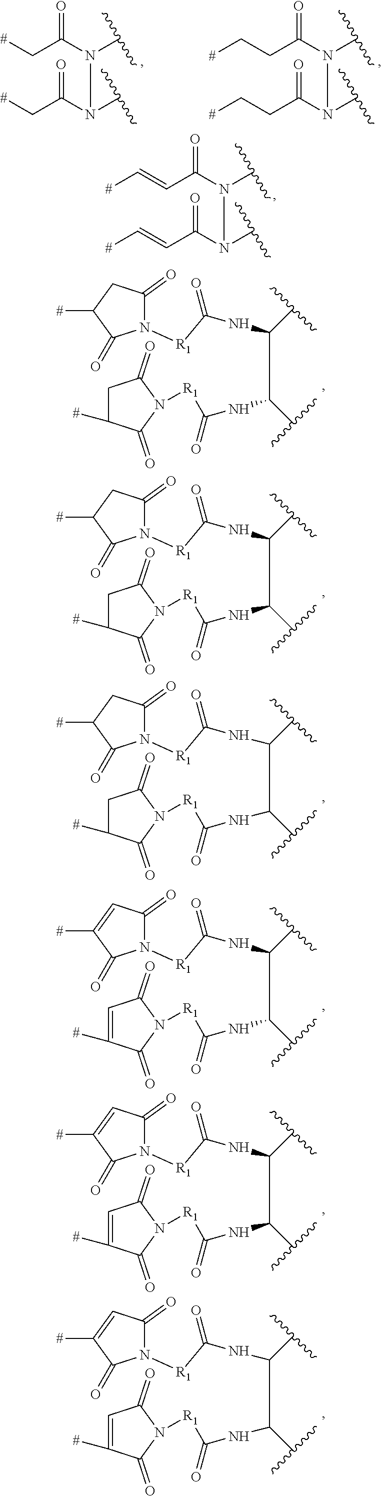 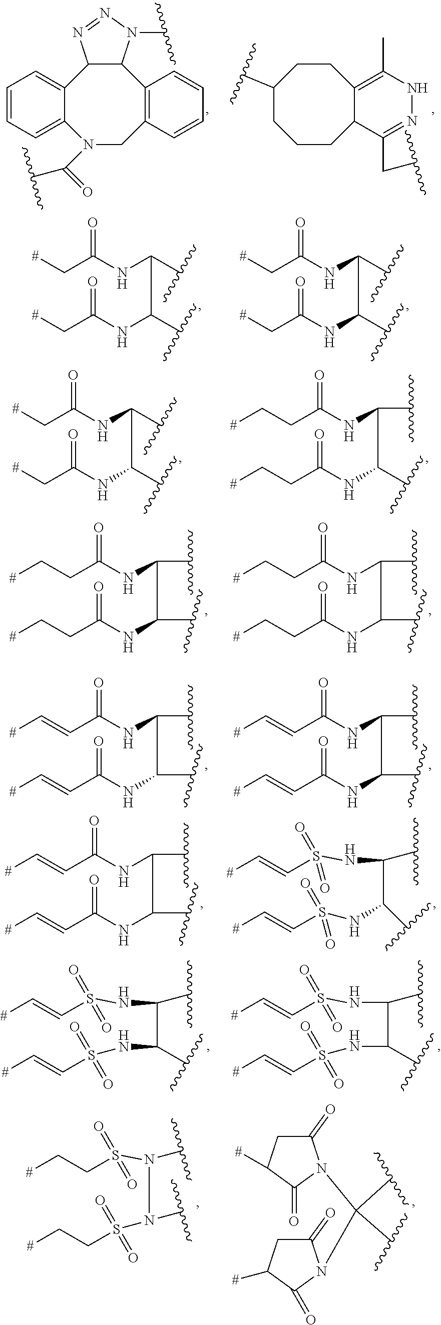 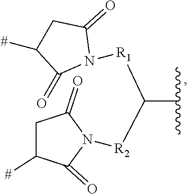 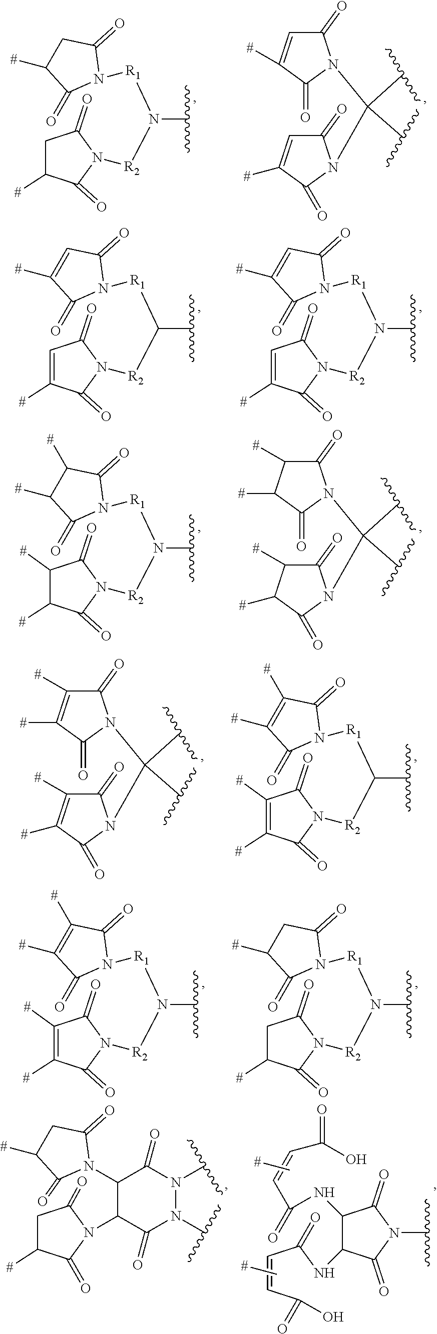 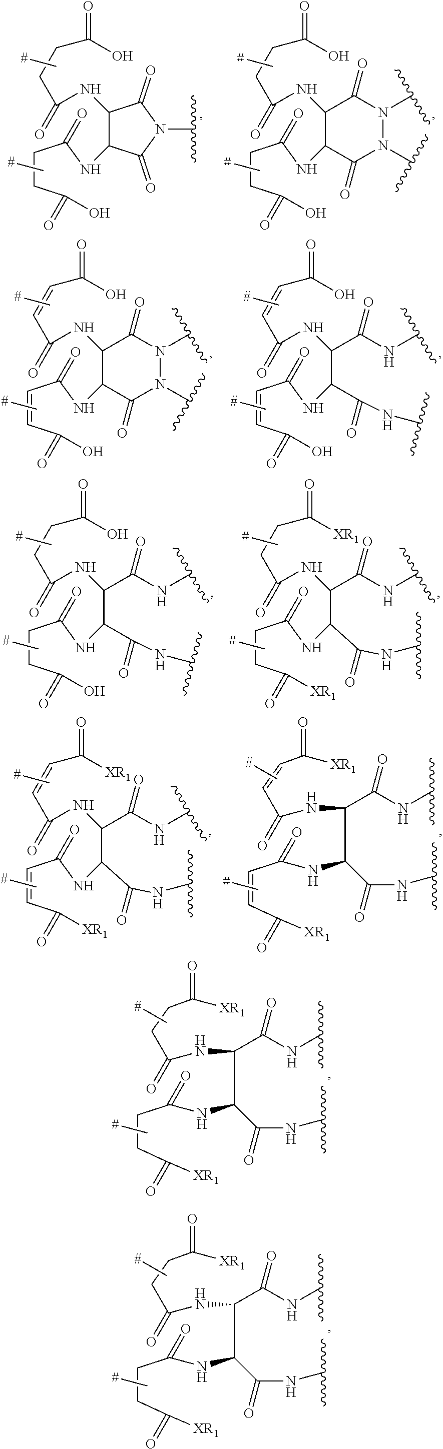 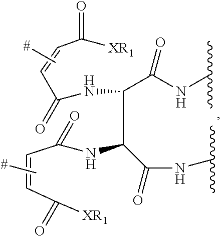 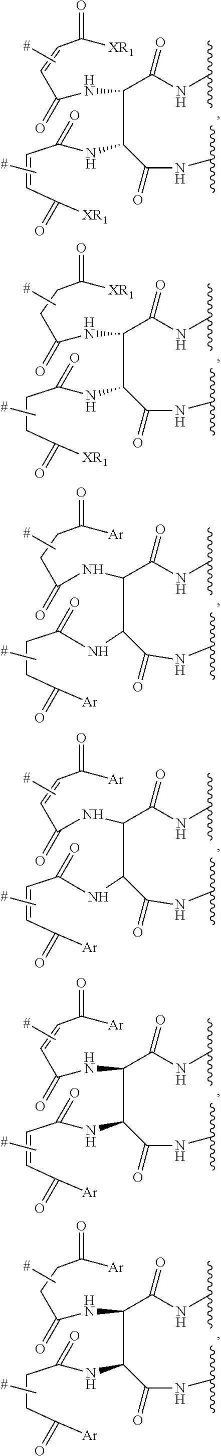 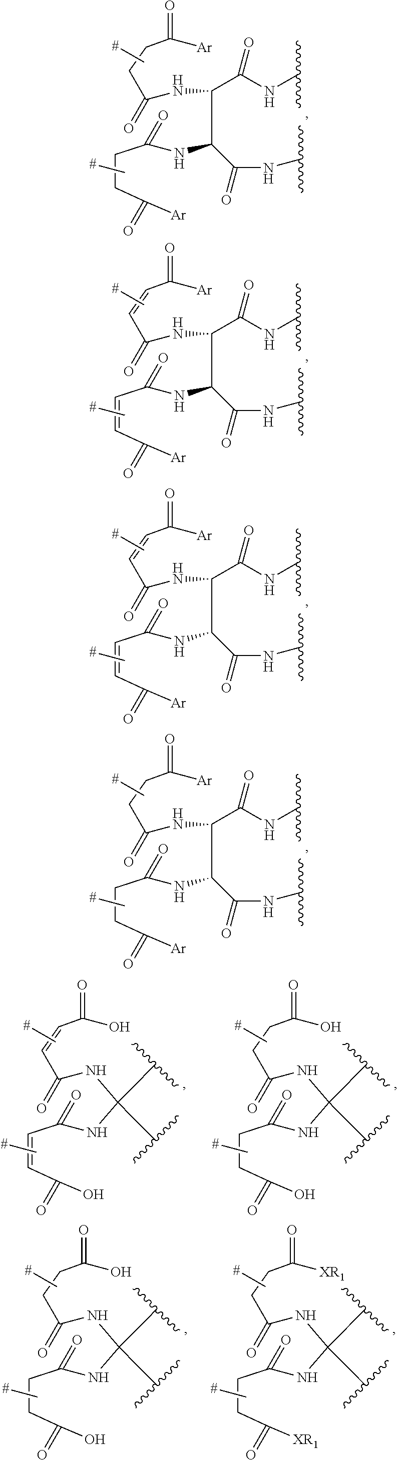 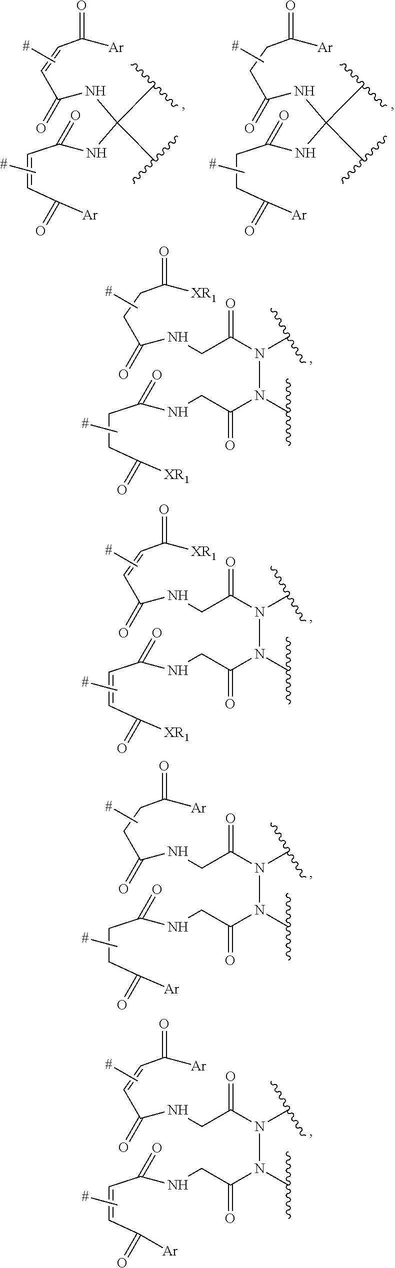 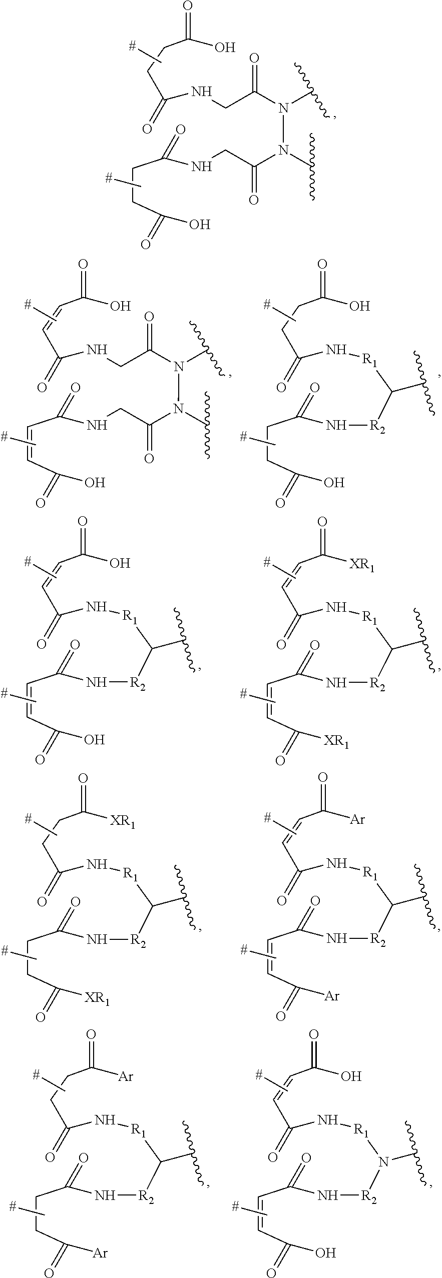 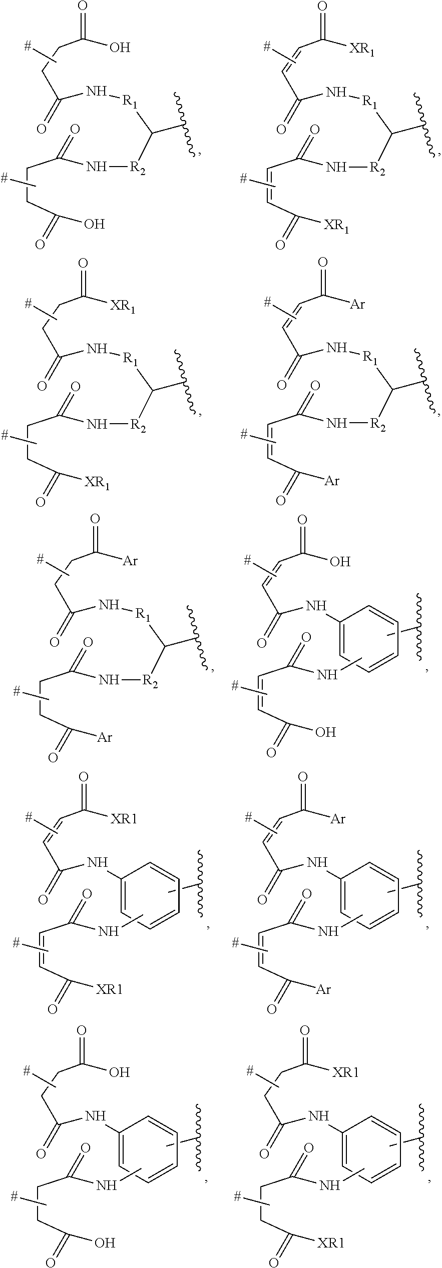 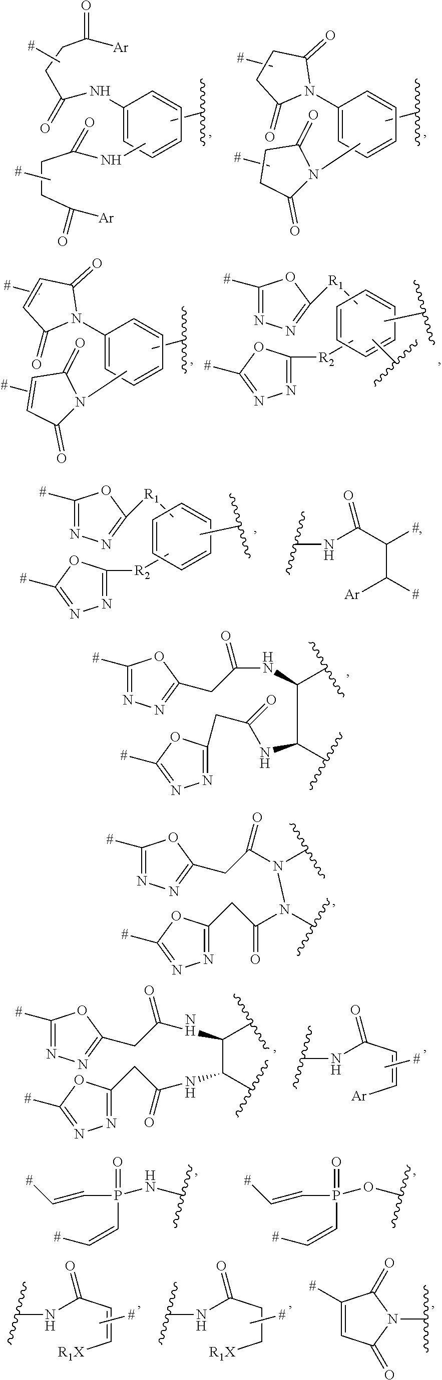 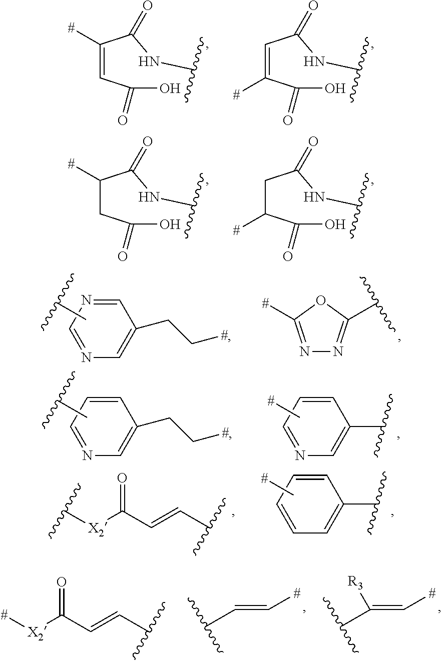
wherein “ [symbol] ”is a site that links a drug or a site of linker Li or L₂; “#” is a site that links a S (thiol), O (phenol), NH (amino), CHO (aldehyde), C(═O) (ketone), C(O)(NH) (amide) and C(O)(OH) (carboxylate) of an antibody; wherein R₁ and R₂ are H, C₁-C₆ of alkyl or a peptide containing 1-4 units of aminoacids; X, X₁′ and X₂′ are O, NH, S, CH₂; the conneting bond “—” in the middle of the two atoms means it can link either one of the two atoms, Ar is an aromatic group;
E₁ and E₂ are a joint group that link two reactivable groups, and are independently selected from CH, CH₂, CH—CH, NH, NHNH, N(R₃), N(R₃)N(R₃′), N═N, N—N, P, P(═O), S, Si, C₂-C₈ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing 1-4 units of aminoacids, or one of the following structures: 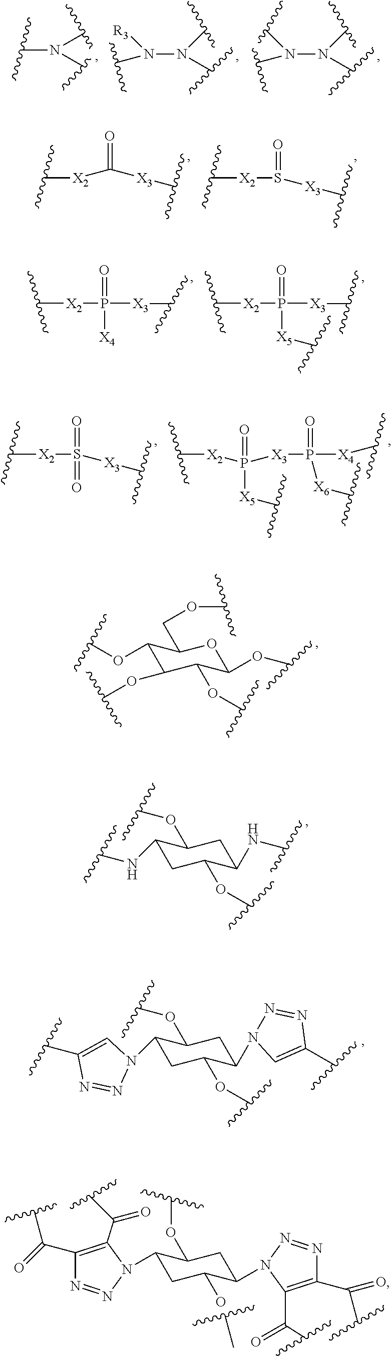 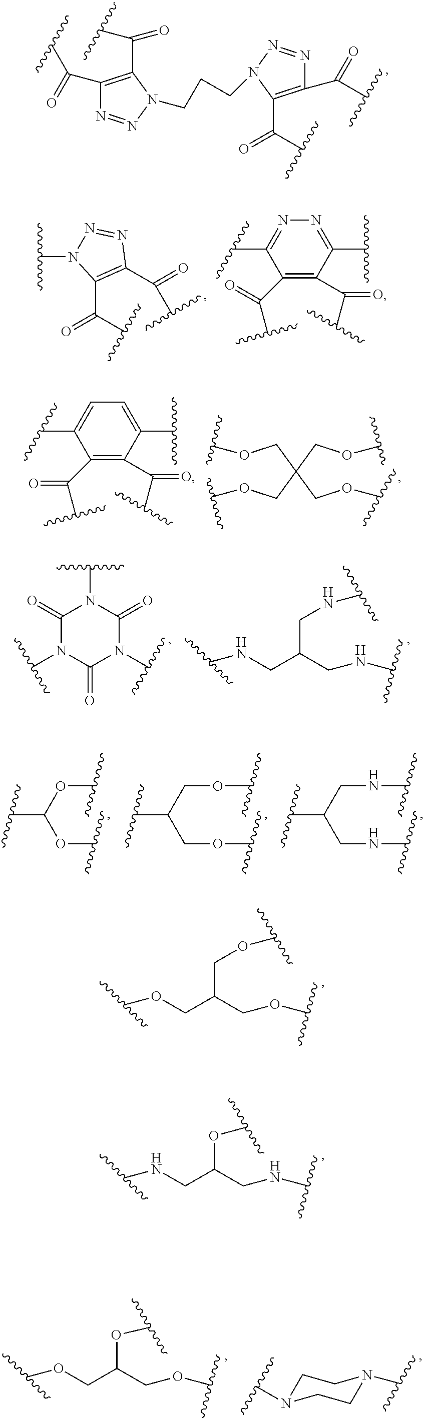 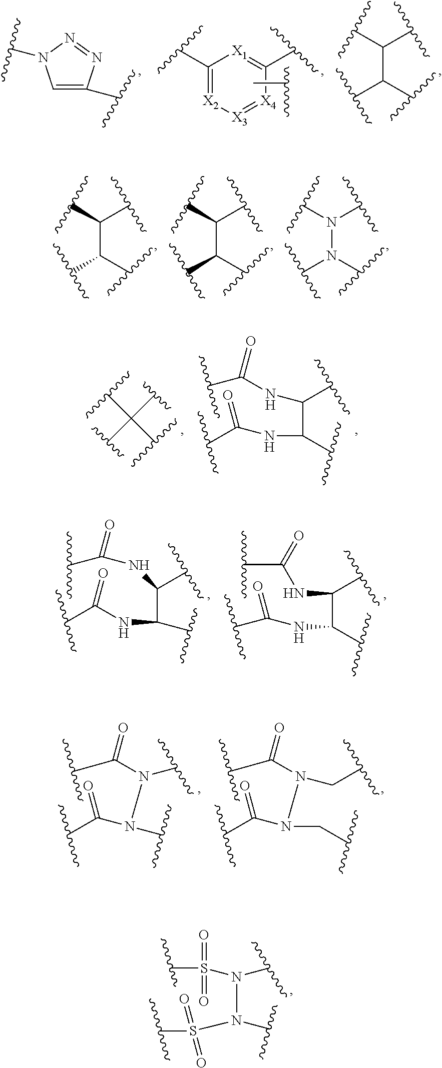
wherein [symbol] is the site of linkage; X₁, X₂, X₃, X₄, X₅, or X₆, are independently selected from NH; NHNH; N(R₃); N(R₃)N(R₃′); O; S; C₁-C₆ of alkyl; R₃ and R₃′ are H, C₁-C₆ of alkyl; In addition, E₁ can be absent, thus La₁ or/and La₂ can directly link to Lv₁′ or Lv₂′;
m₁, m₂, m₃, m₄, m₅, m₆, m₇, m₈, m₉, m₁₀, m₁₁ and m₁₂ are independently 1, 2, 3, 4, 5, 6, 7, 8, 9, or 10 and these numbers may have a decimal; in addition, m₂, m₃, m₈, m₉ and/or m₁₀ can be 0, thus Ld₂—A₂, Ld₃ A₃, Ld₅—A₅, and/or Ld₆—A₆ can be absent;
A₁, A₂, A₃, A₄, A₅ and A₆ are independently a functional small molecule selected from (1) an affinity ligand: cell-penetrating peptide (CPP), including a ligand for bombesin receptor/neurotensin receptor or neuropeptide-Y receptor; (2) and/or a nucleoside antimetabolite/analog; which have either the synergy with D₁ or D₂, or enhanced affinity for mAb.

2. The antibody drug conjugate according to claim 1, wherein the functional small molecule, A₁, A₂, A₃, A₄, A₅ and A₆ are independently selected from:
(1) an affinity ligand: 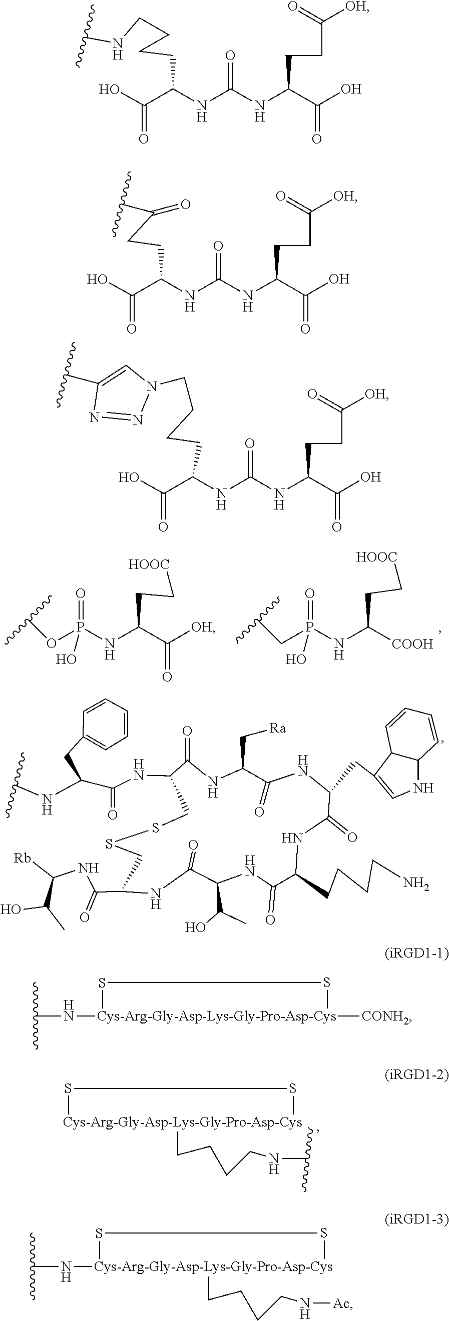 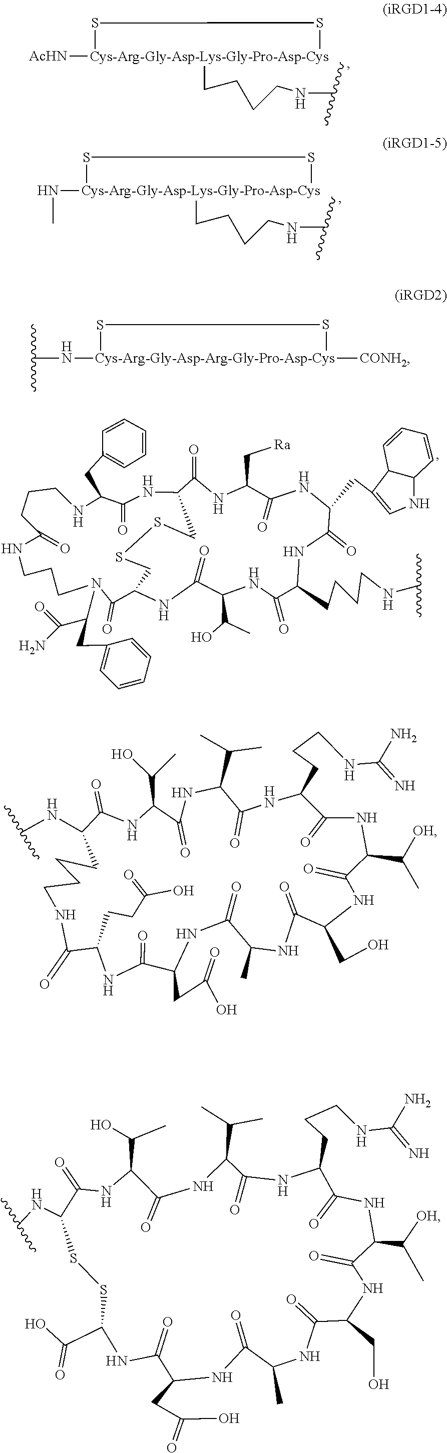 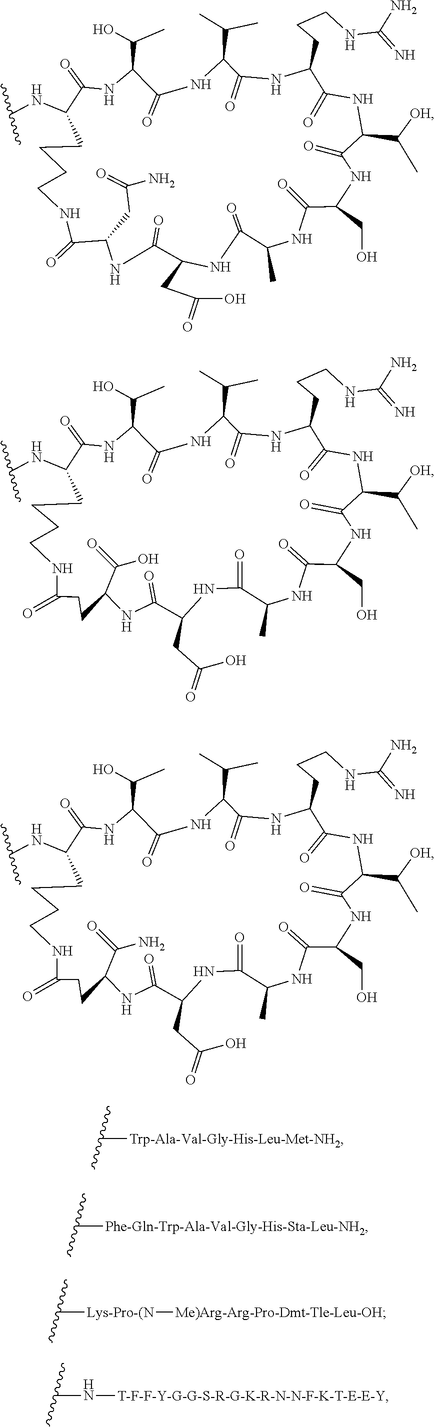 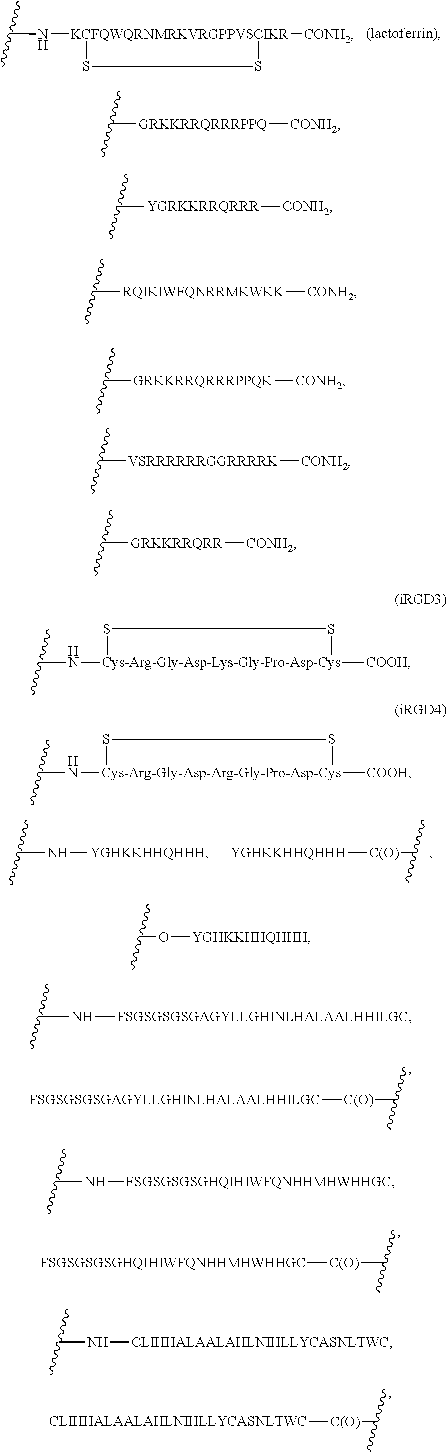 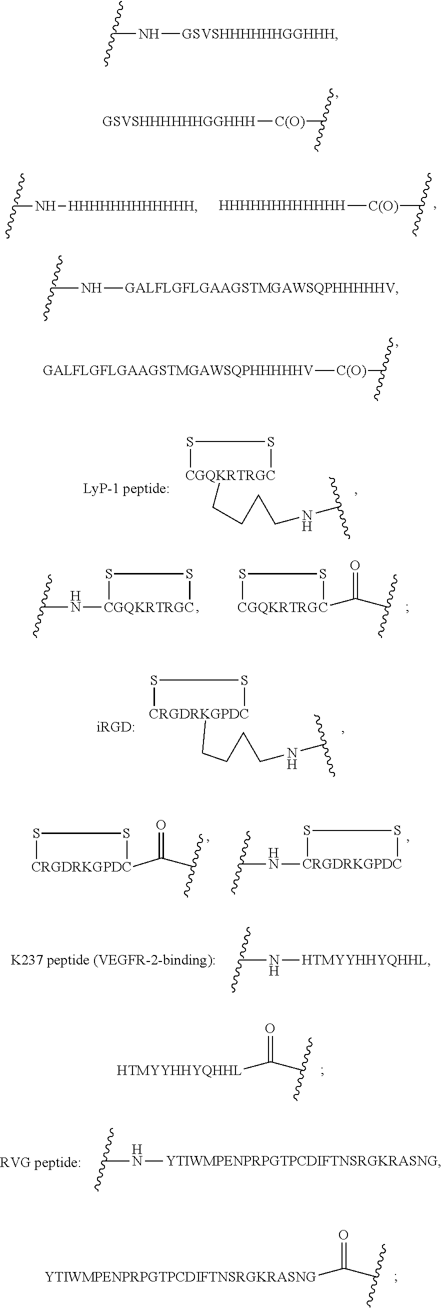 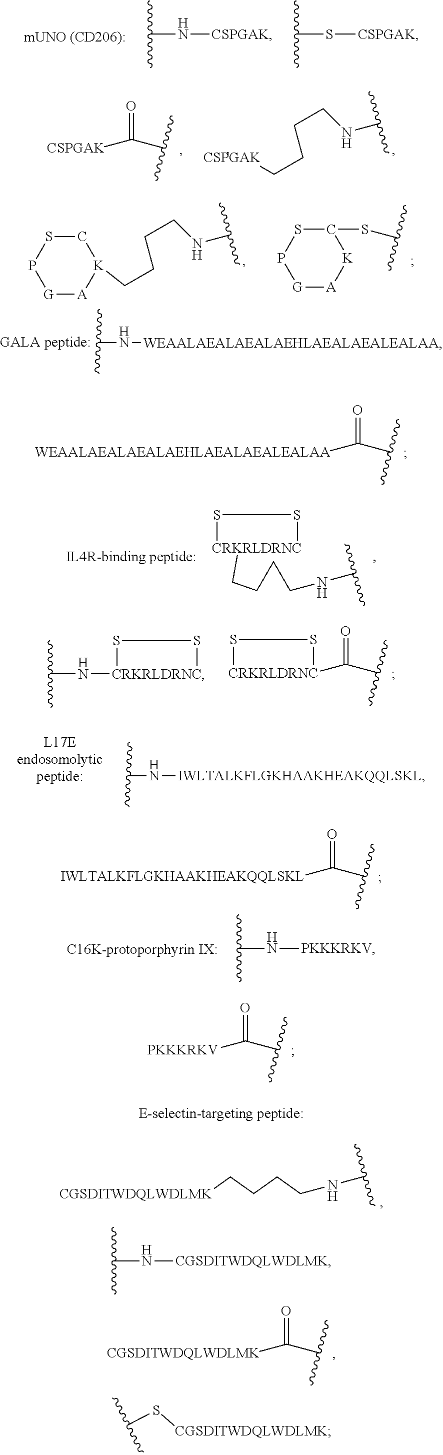 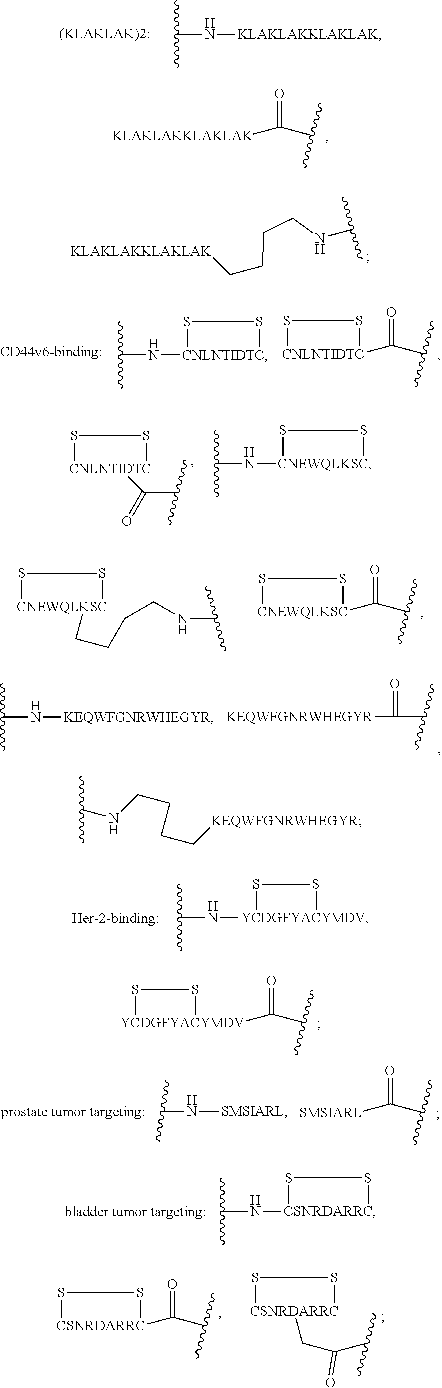 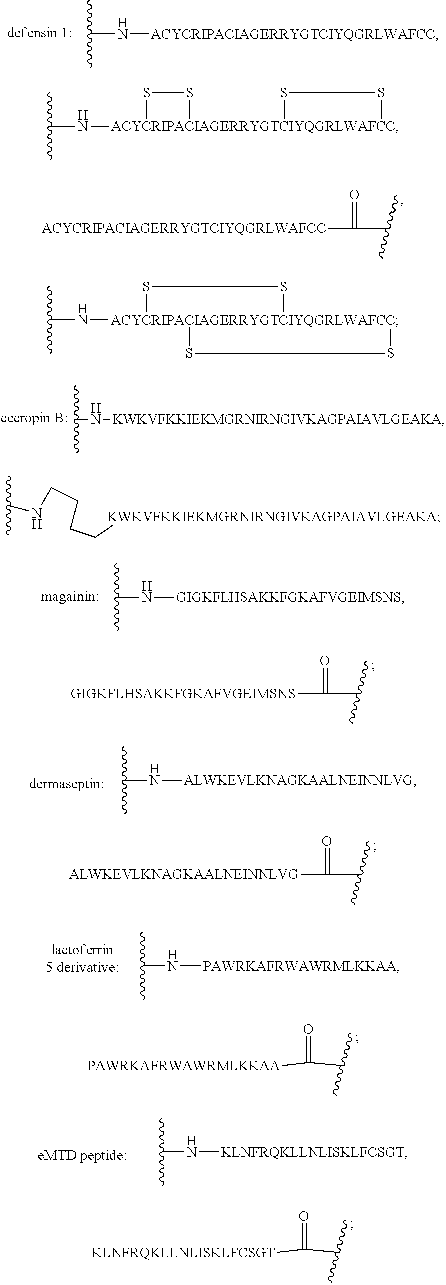 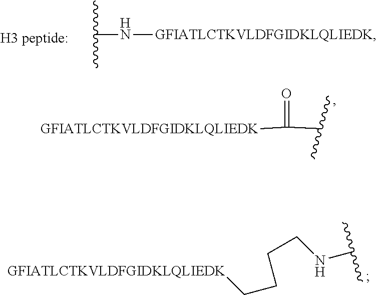 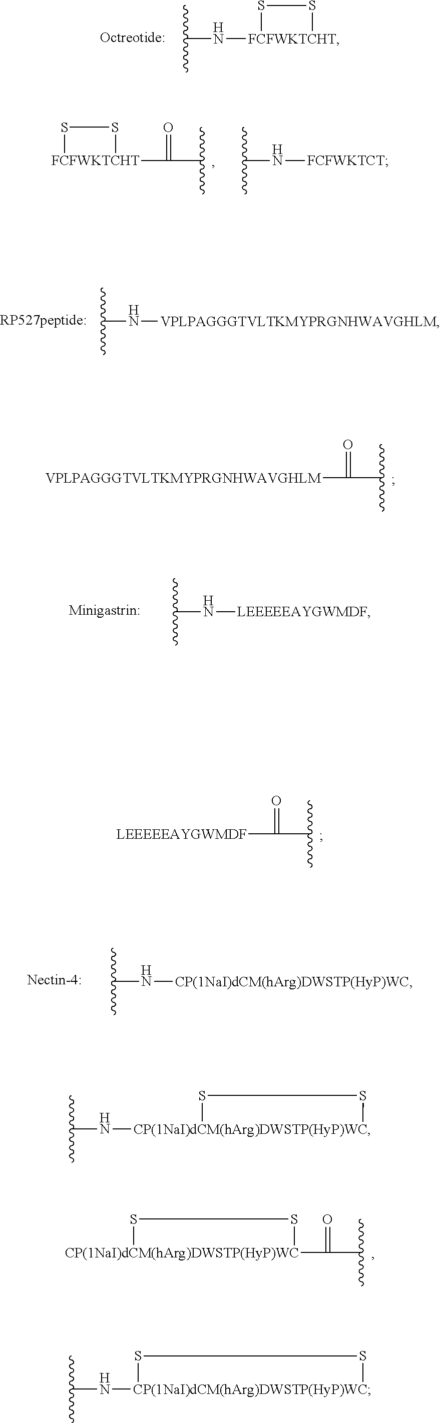 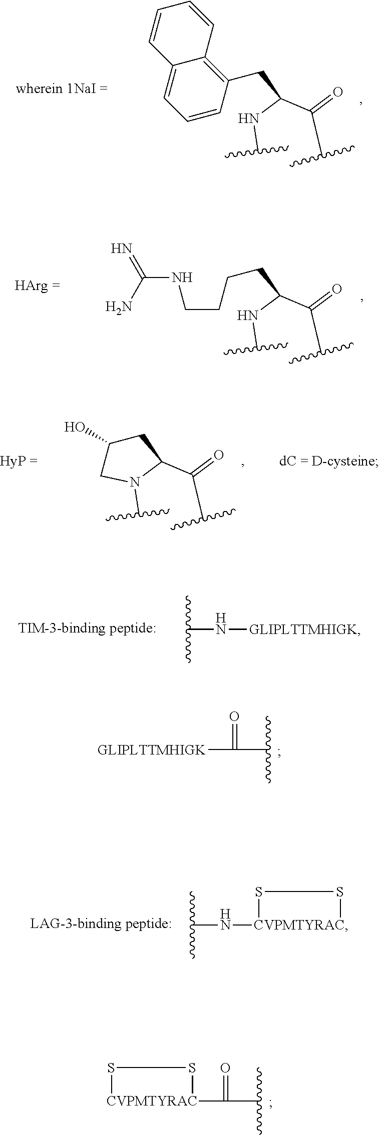 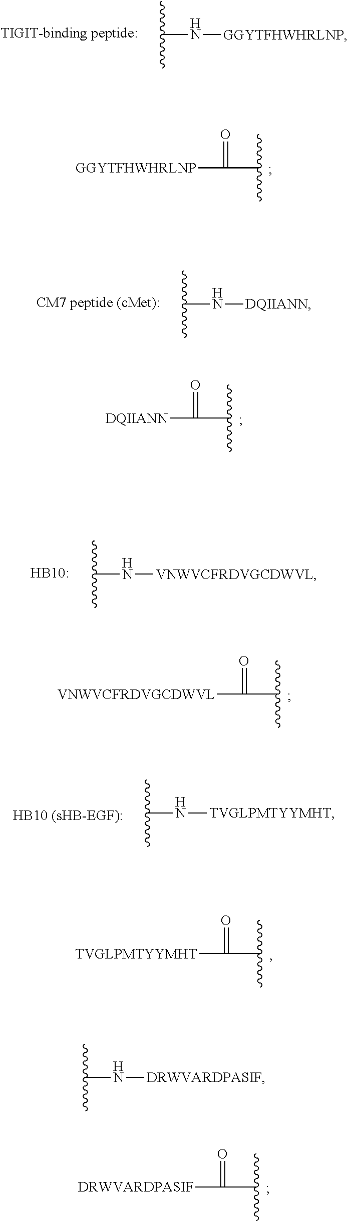 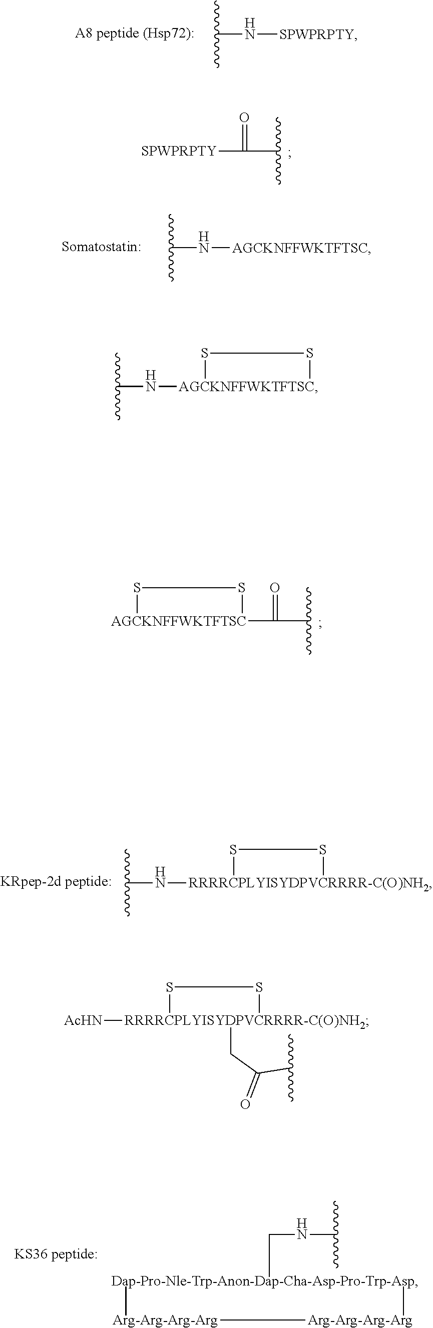
wherein Dap is (S)-2,3-Diaminopropanoic acid, Ne is L-Norleucine, Anon is (S)-2-Aminononanoic acid, Cha is L-Cyclohexylalanine. The others are natural amino acids. 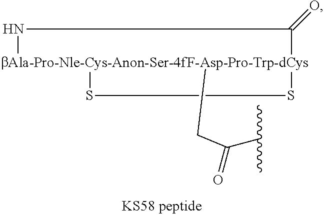
wherein βAla is Beta-alanine, Nle is L-Norleucine, Anon is (S)-2-Aminononanoic acid, 4fF is 4-Fluoro-1-phenylalanine, dCys is D-cysteine; 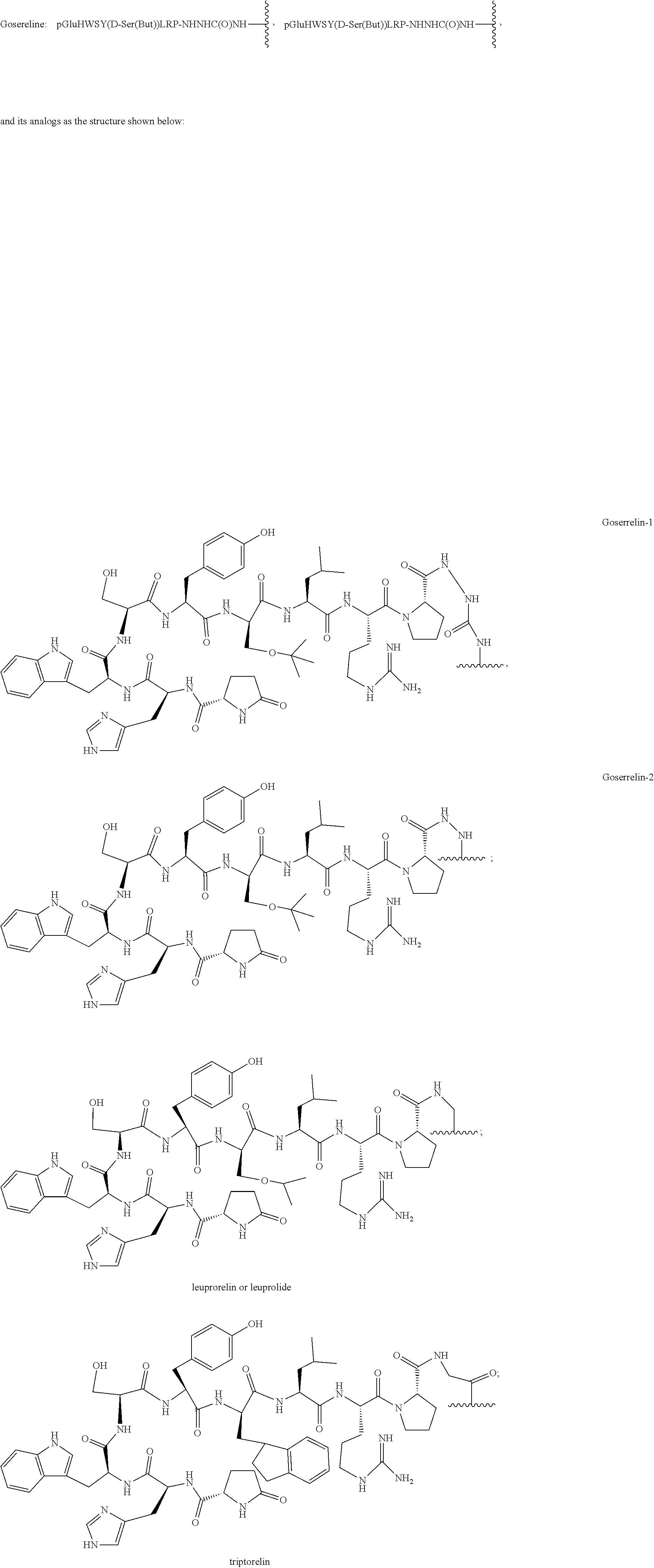 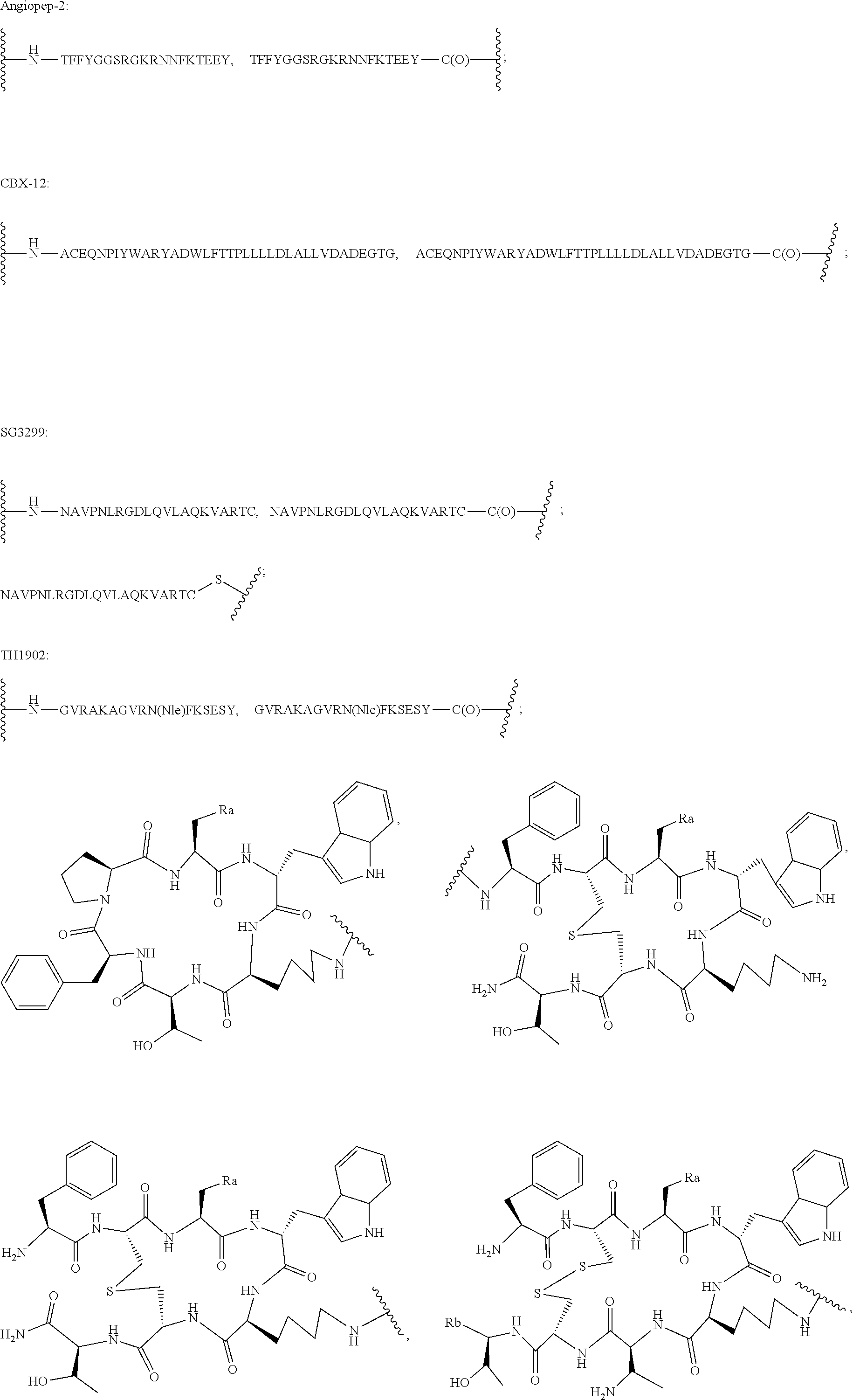 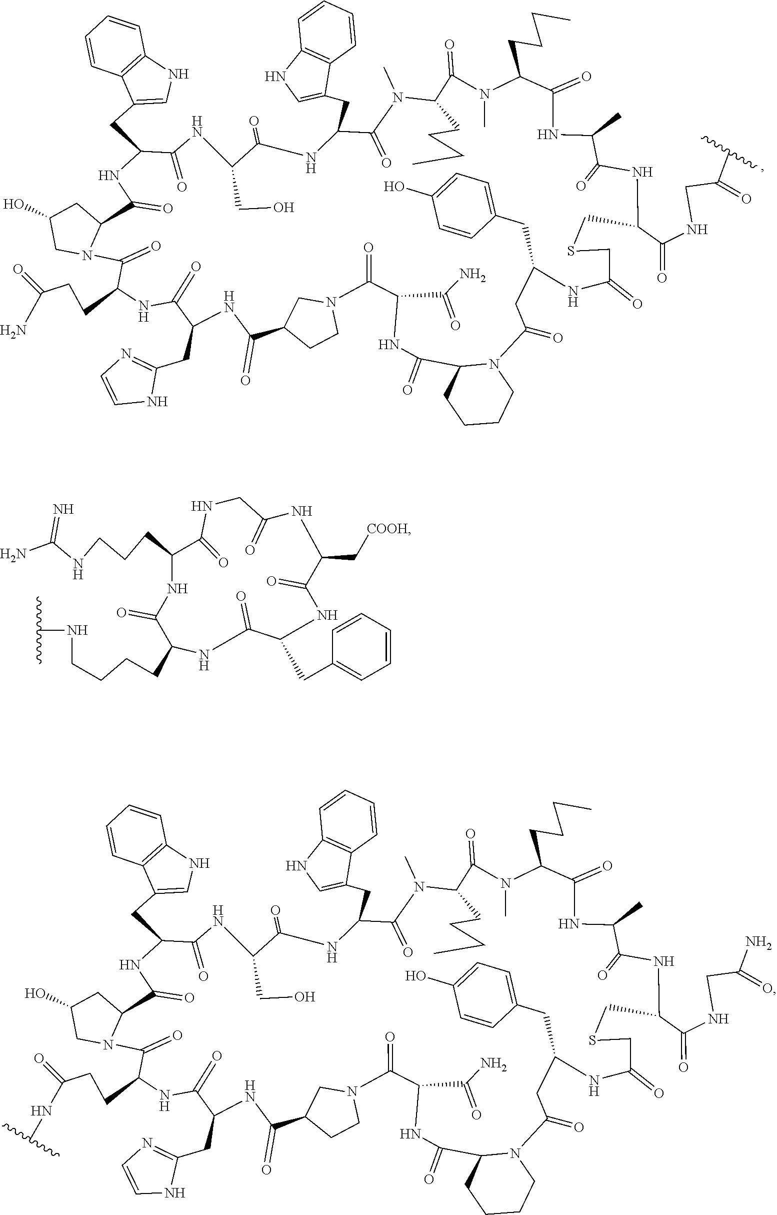 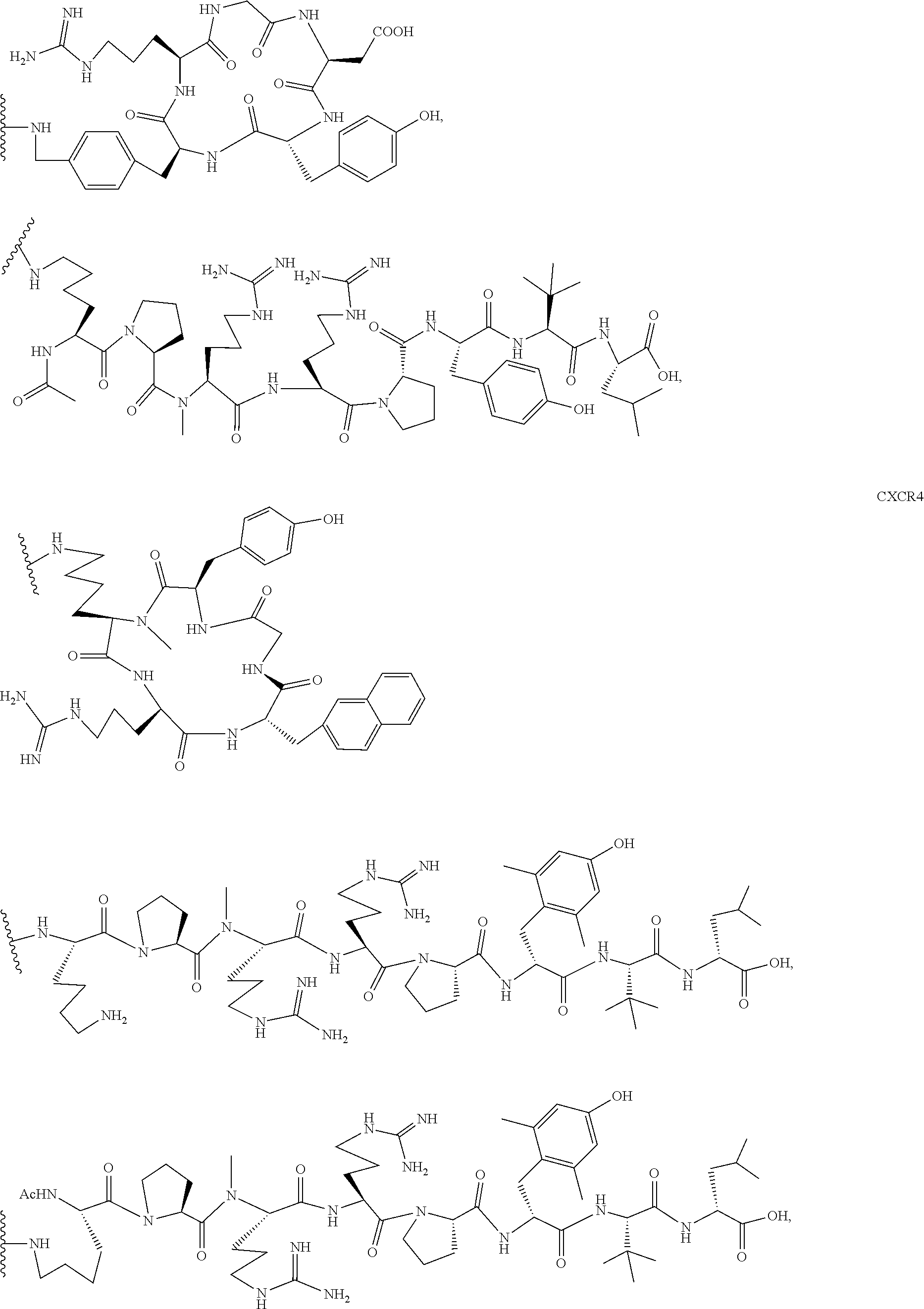 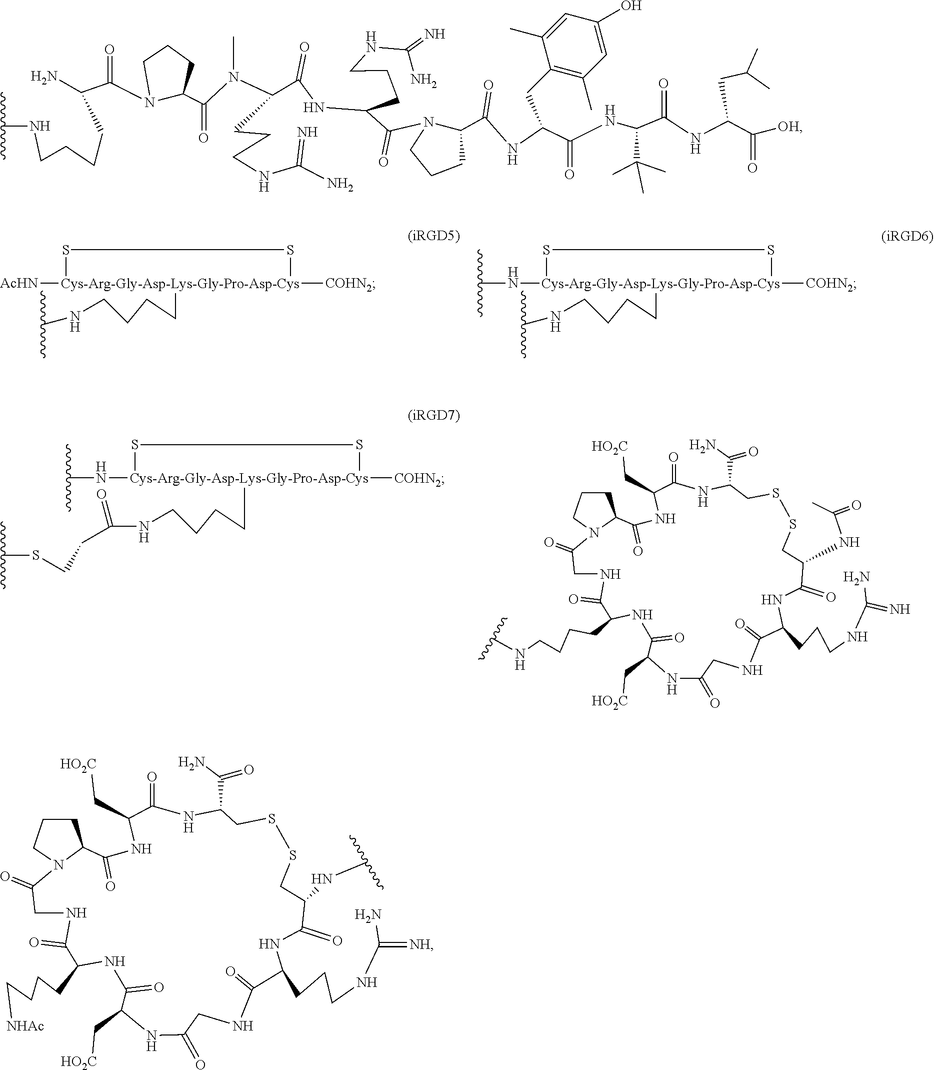 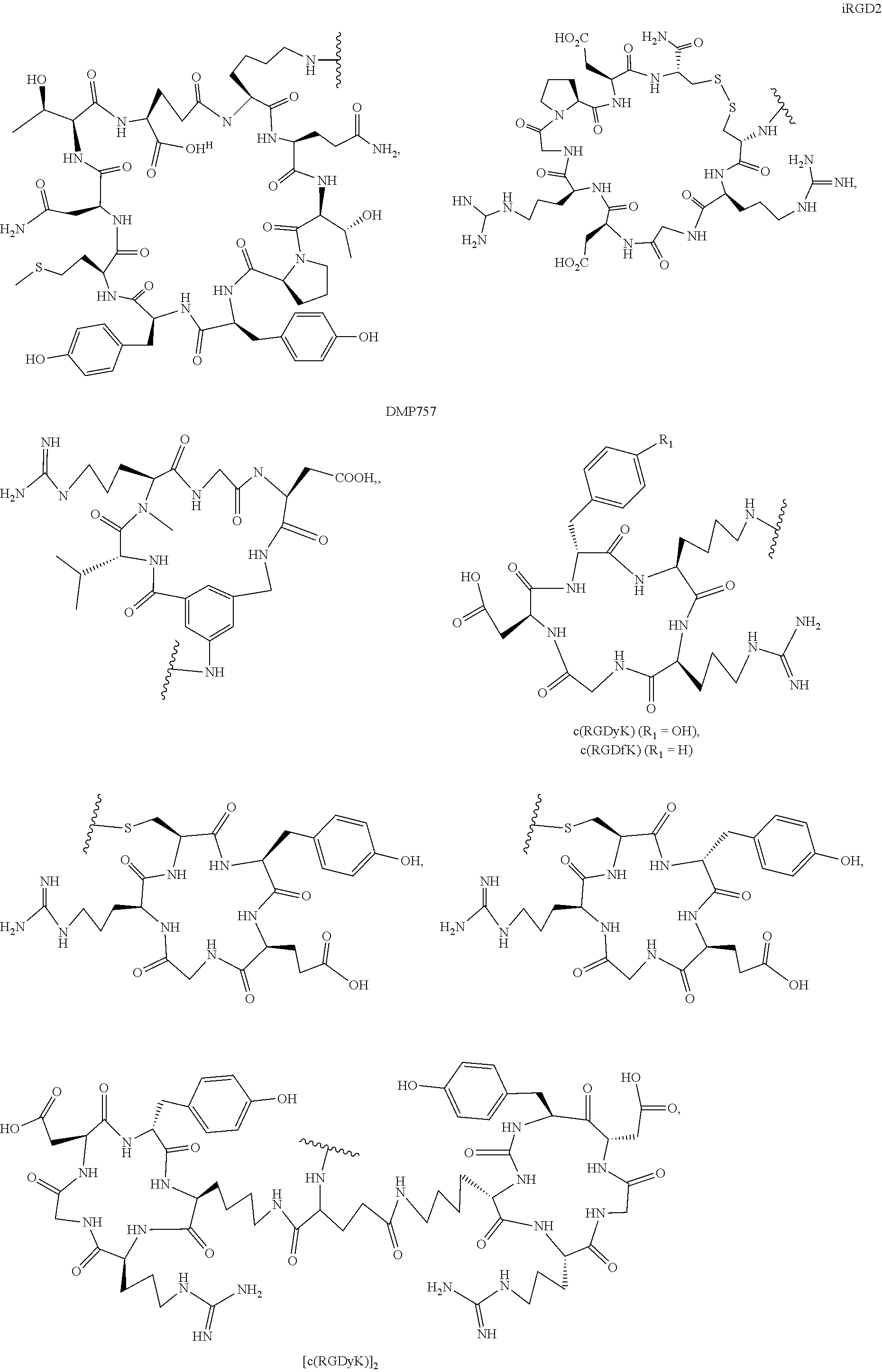 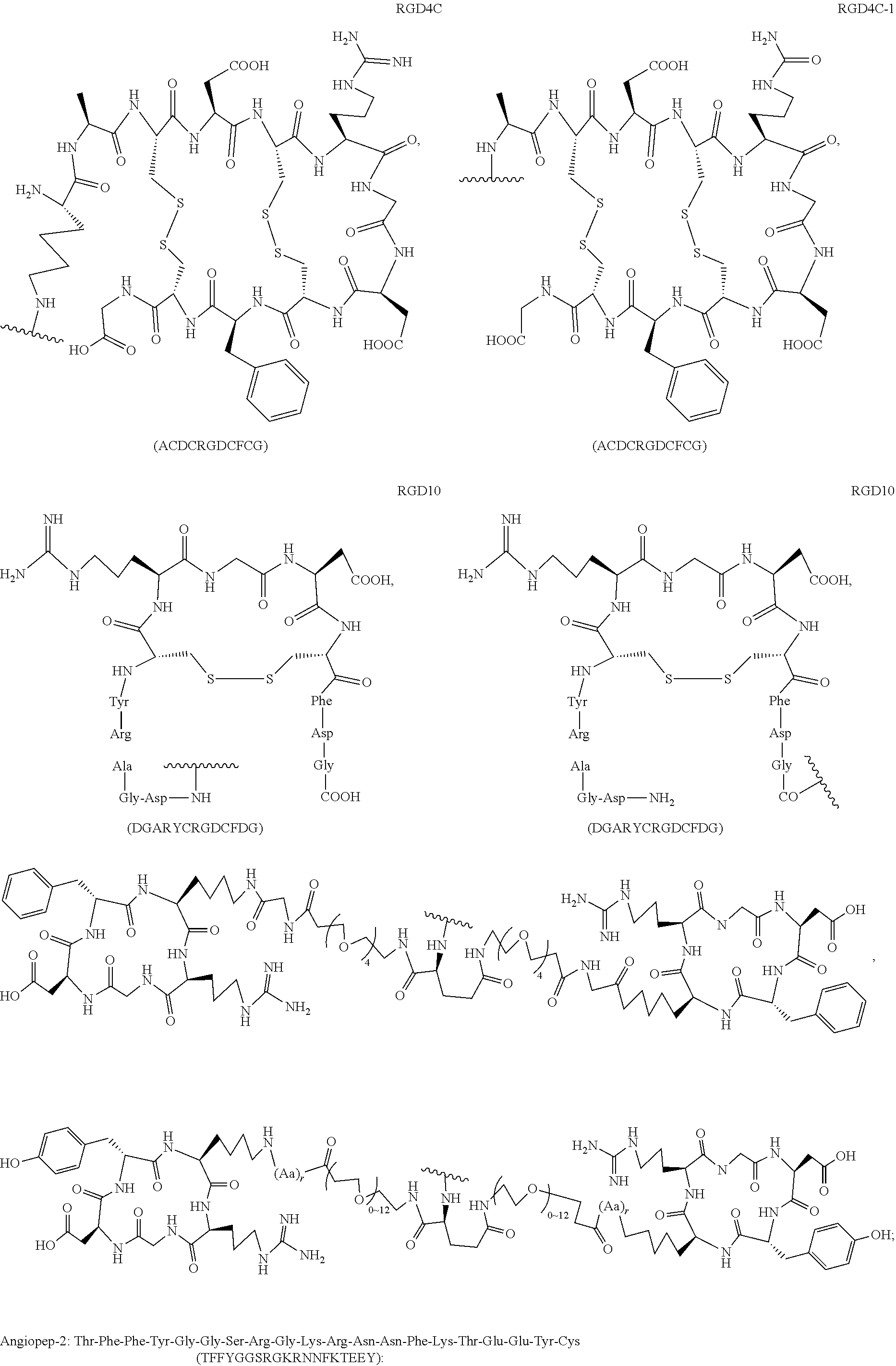 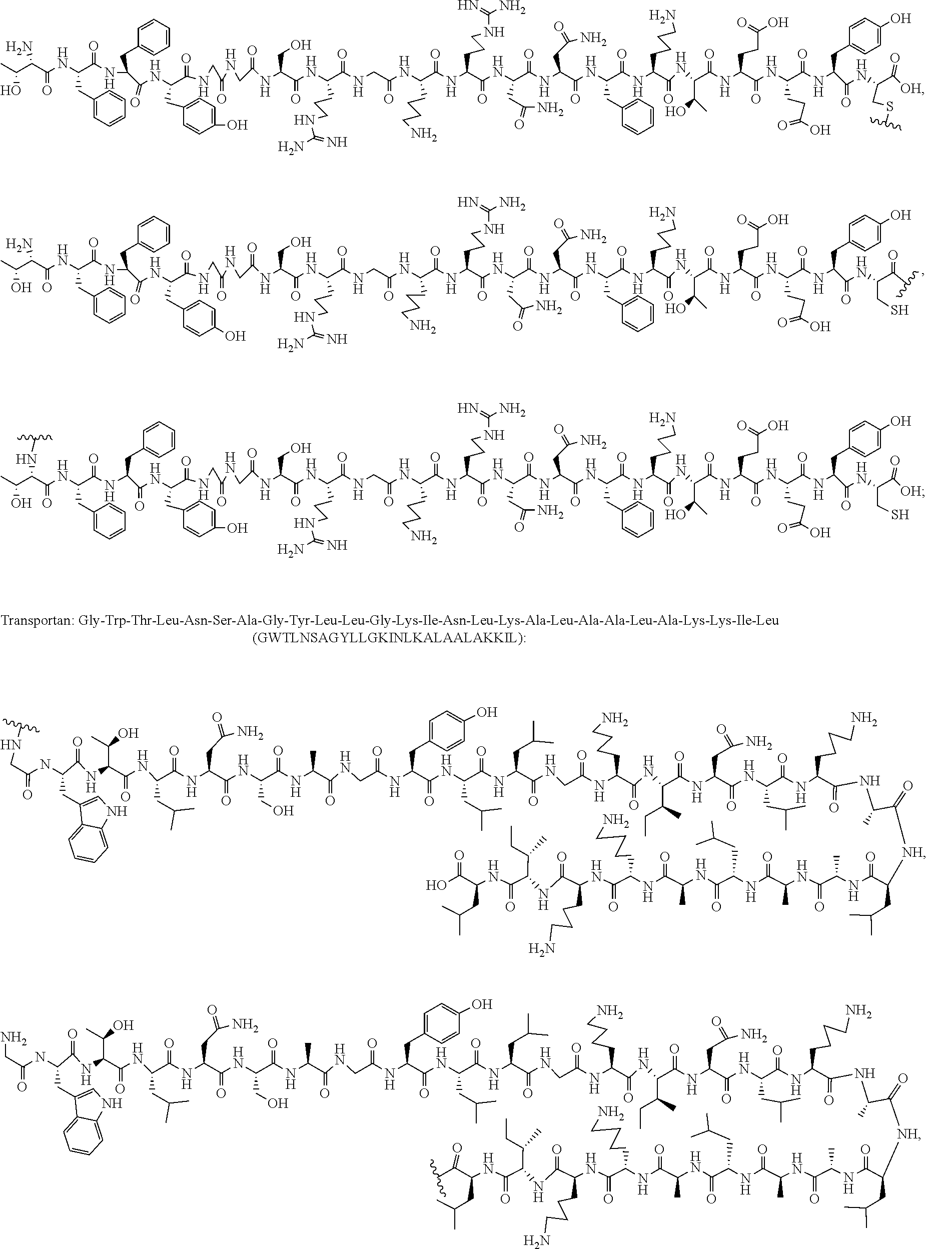 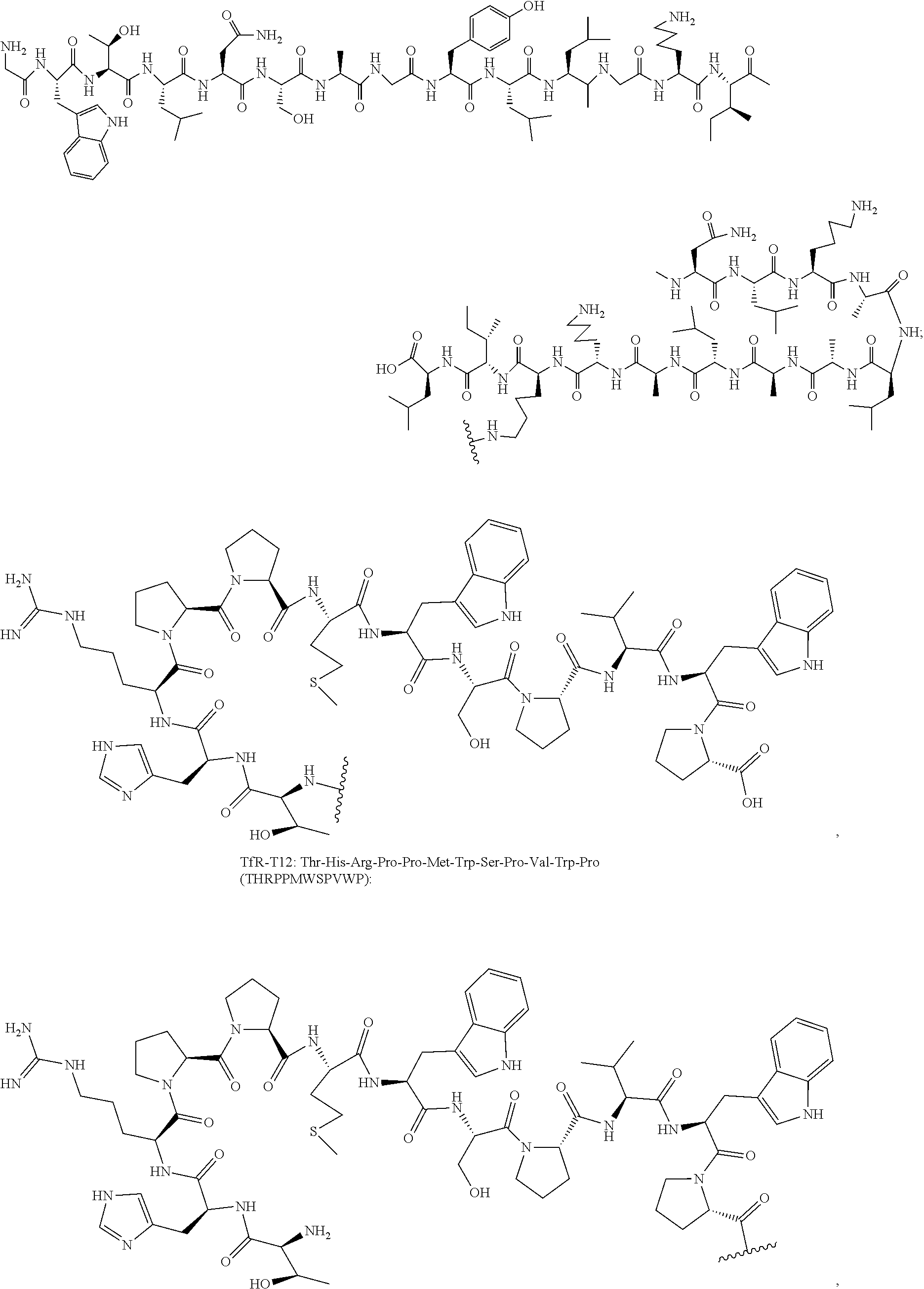 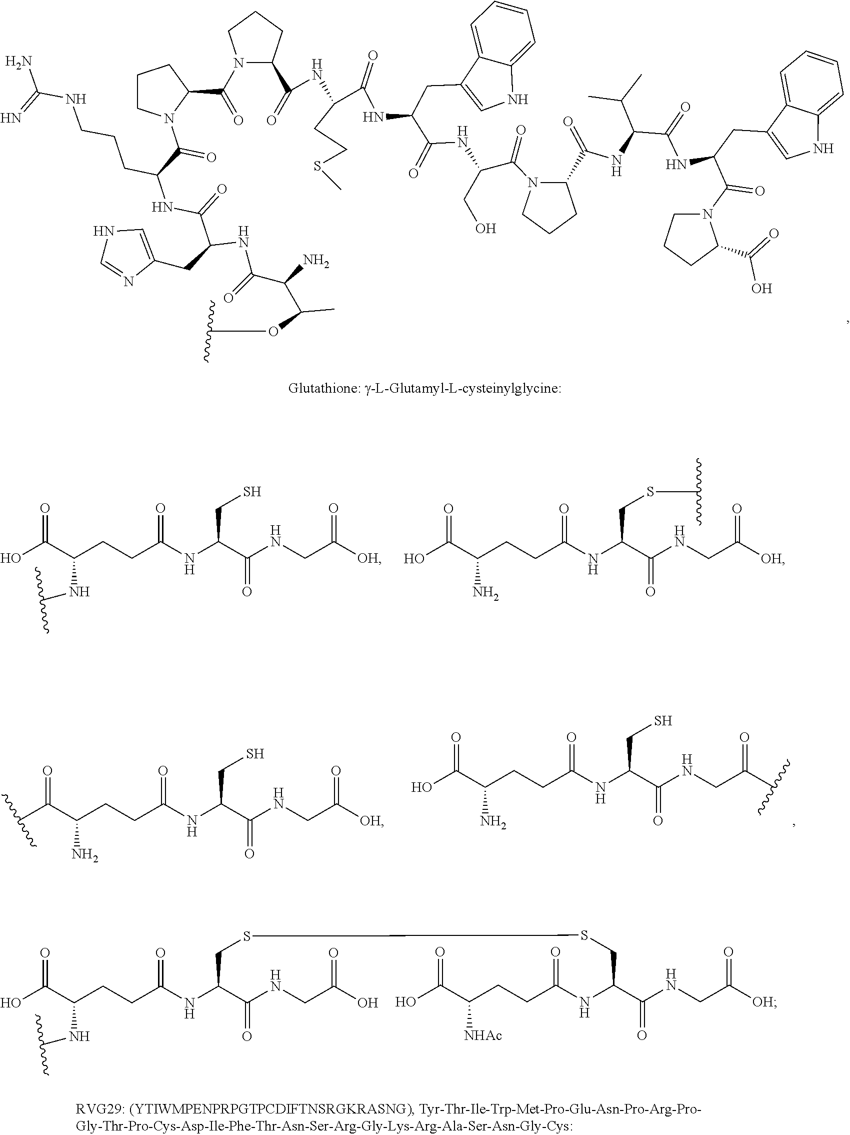 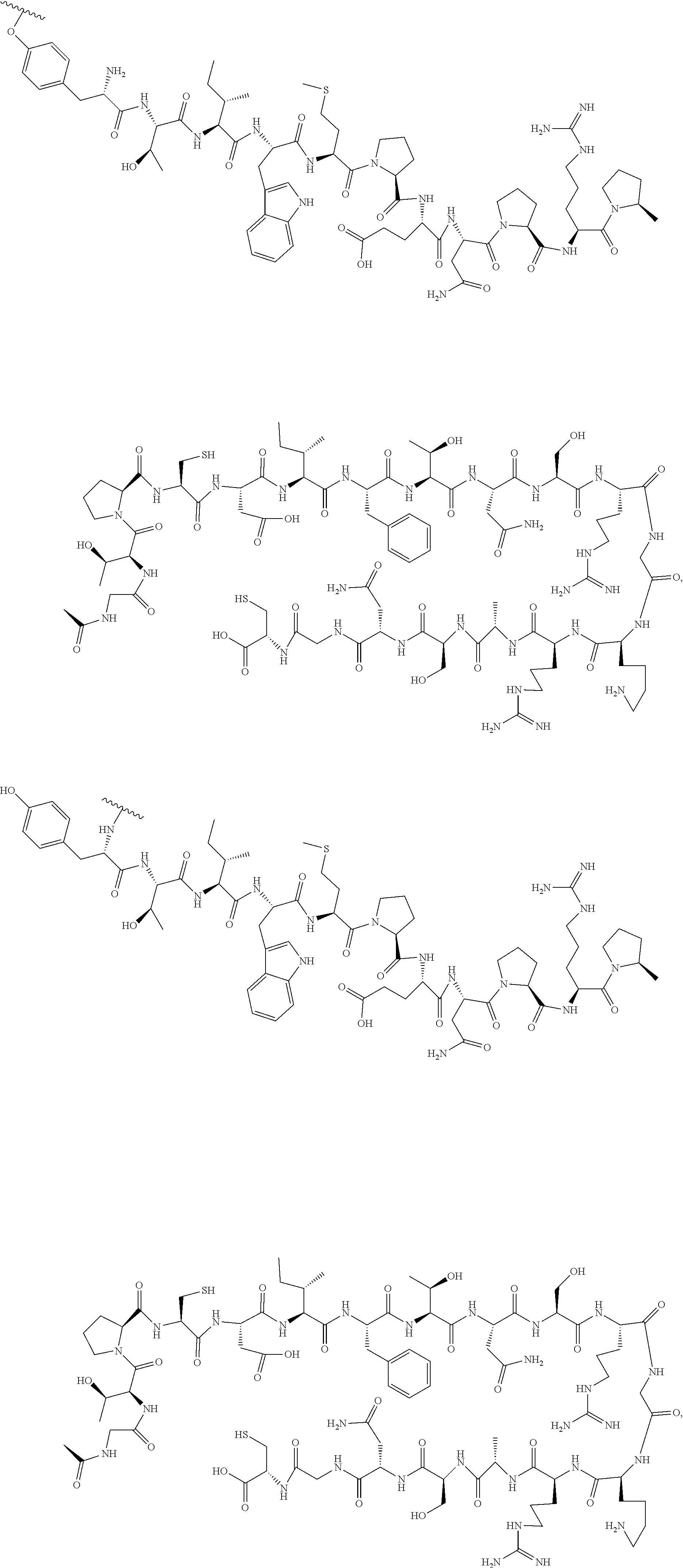 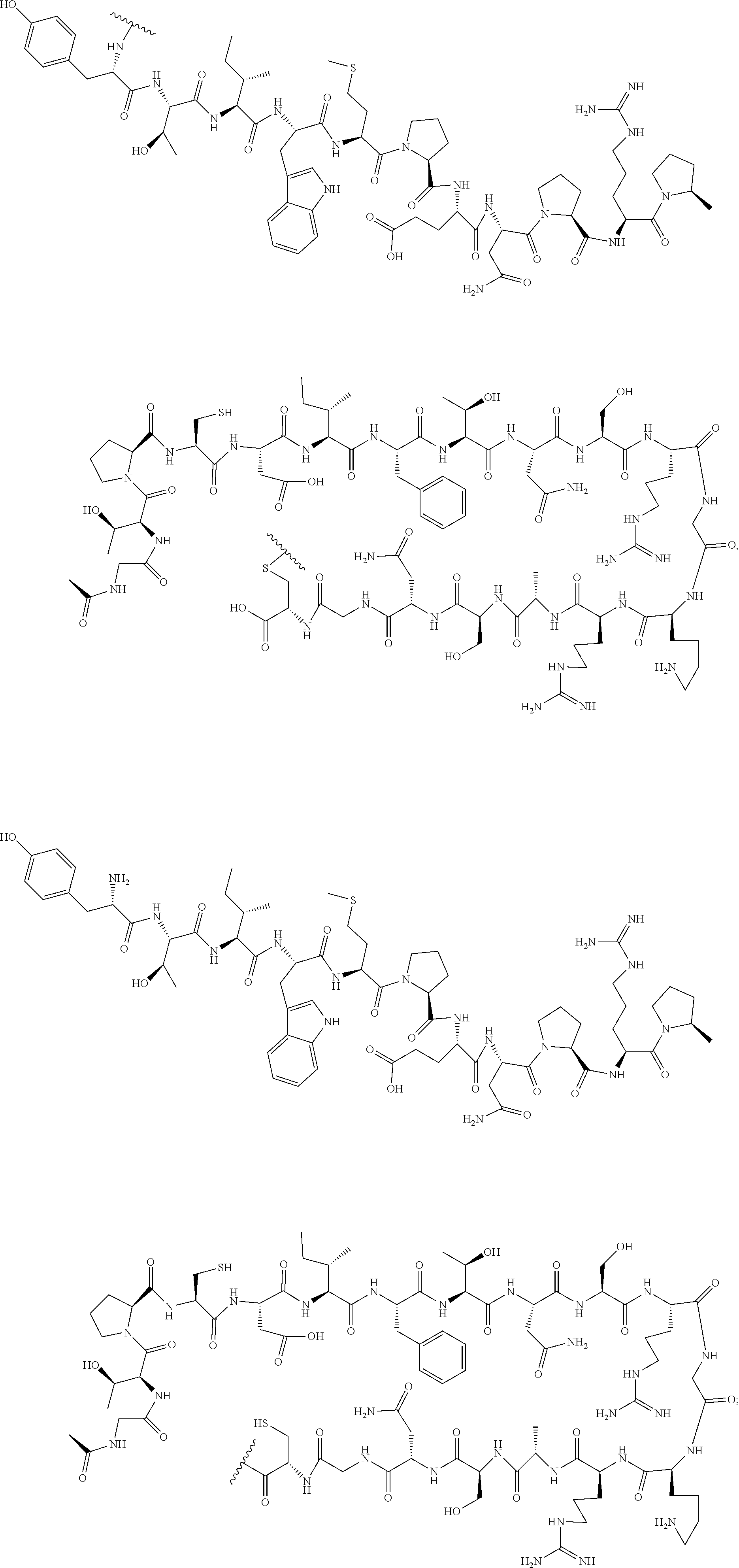 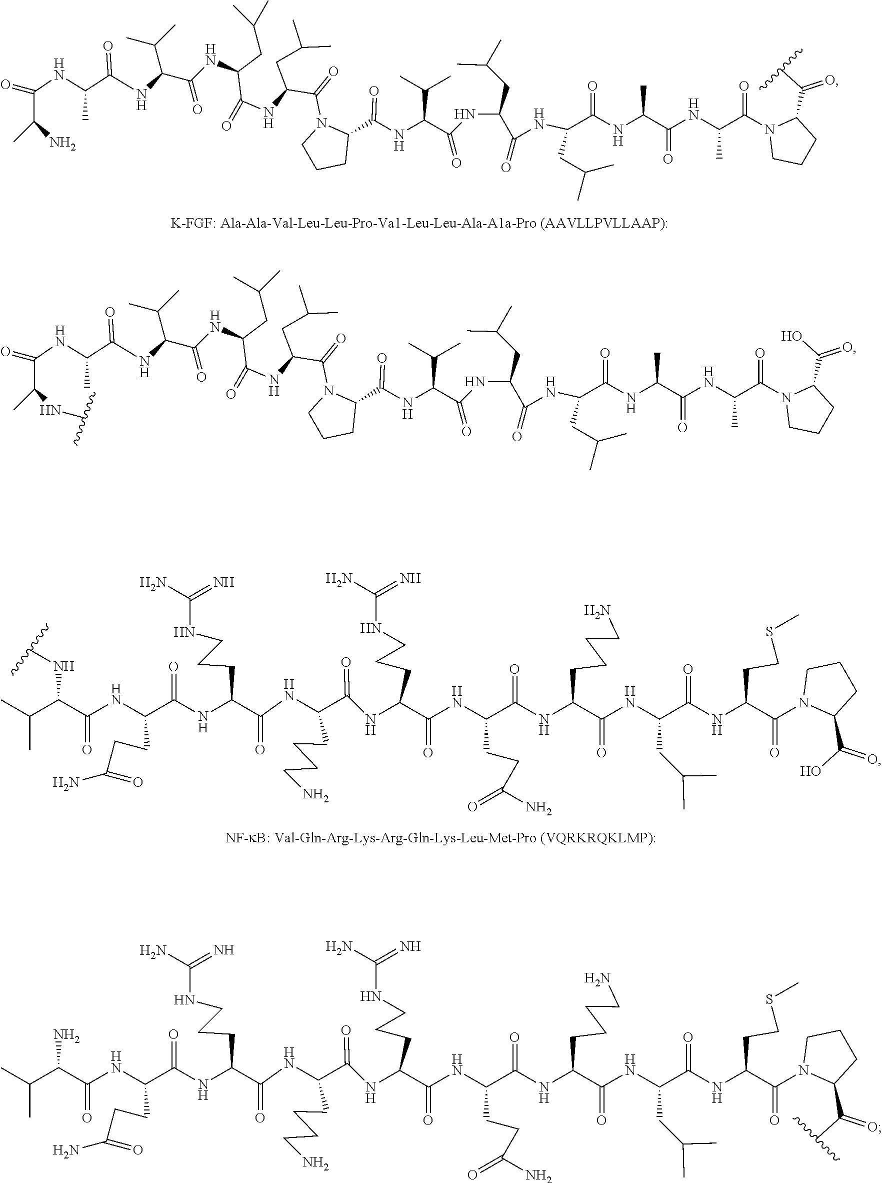 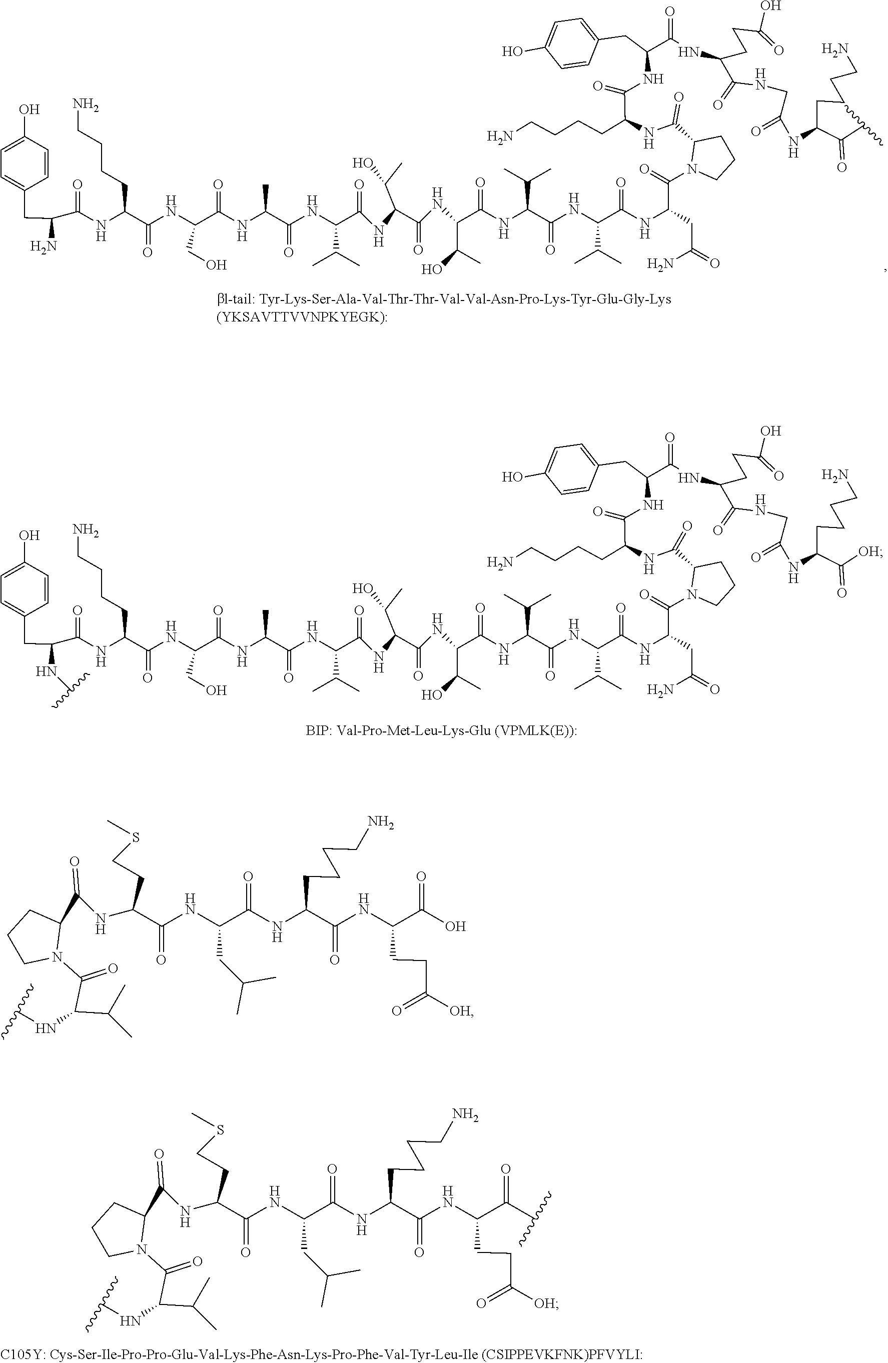 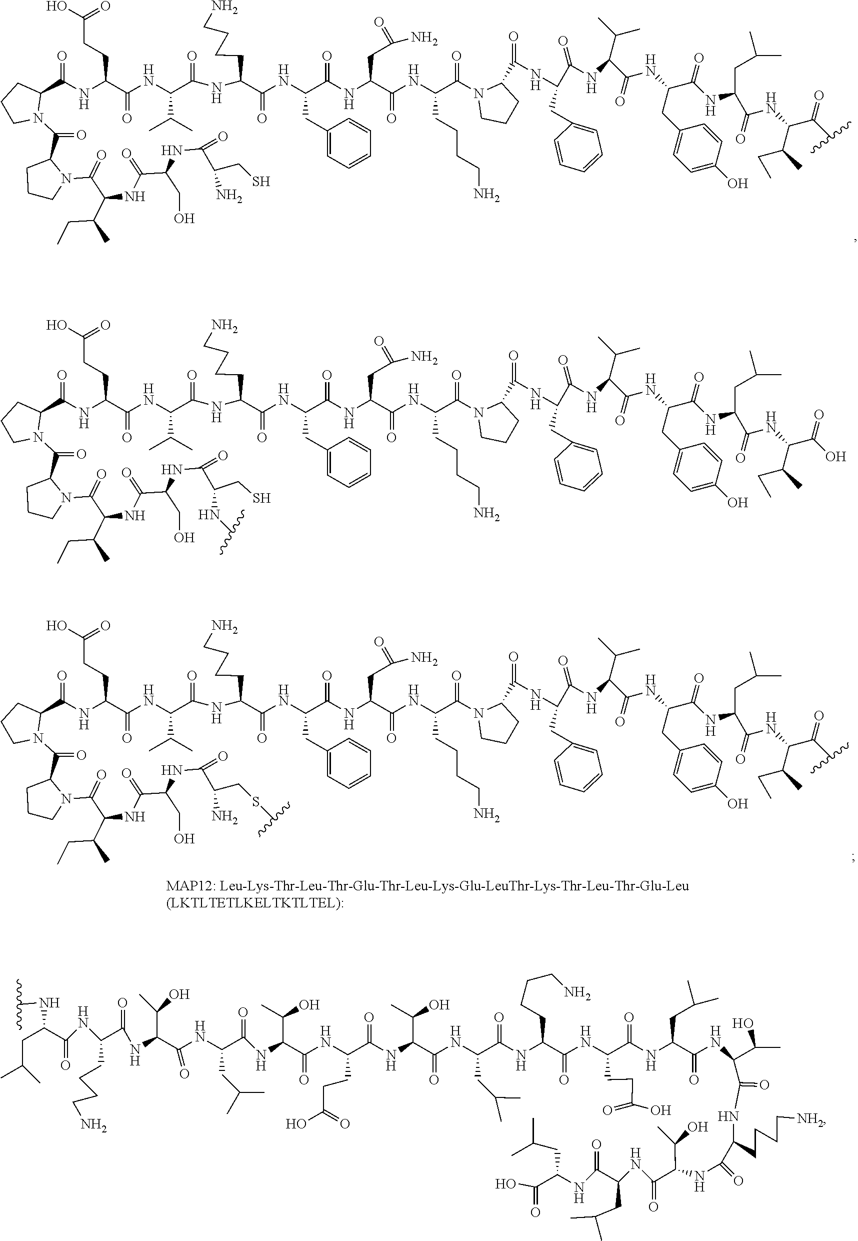 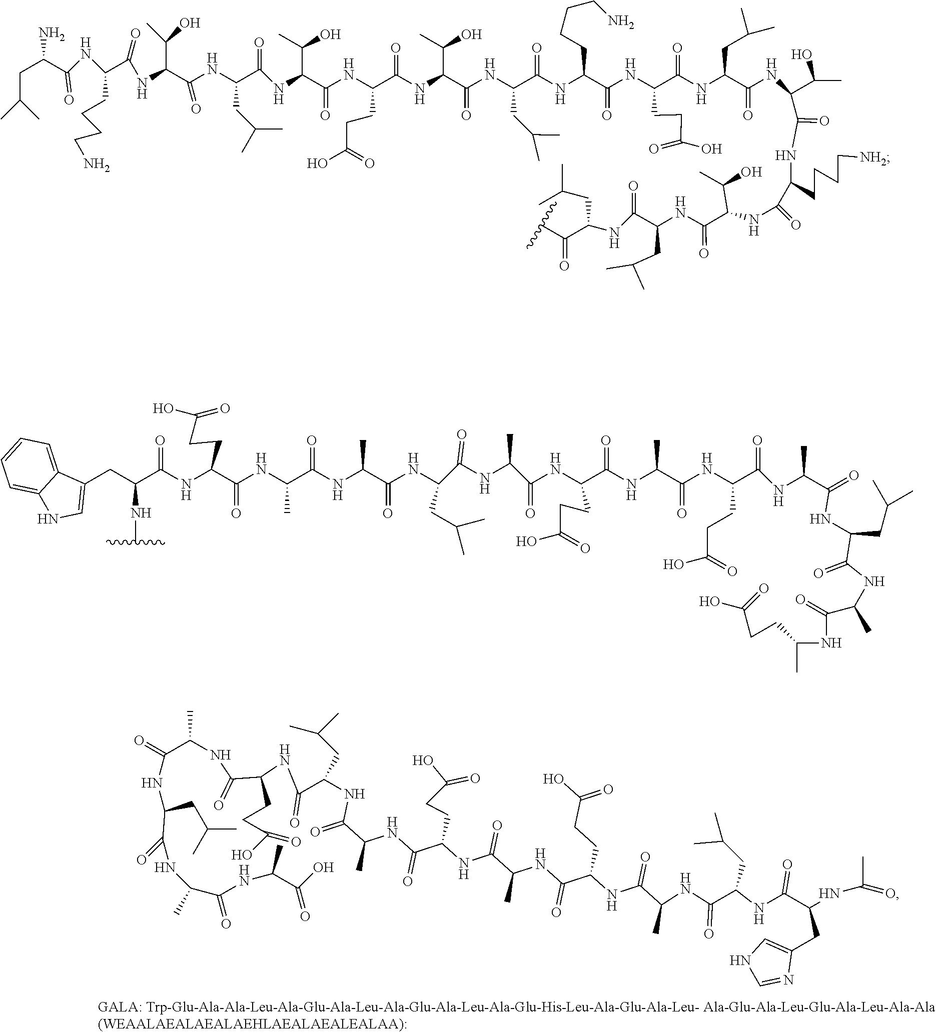 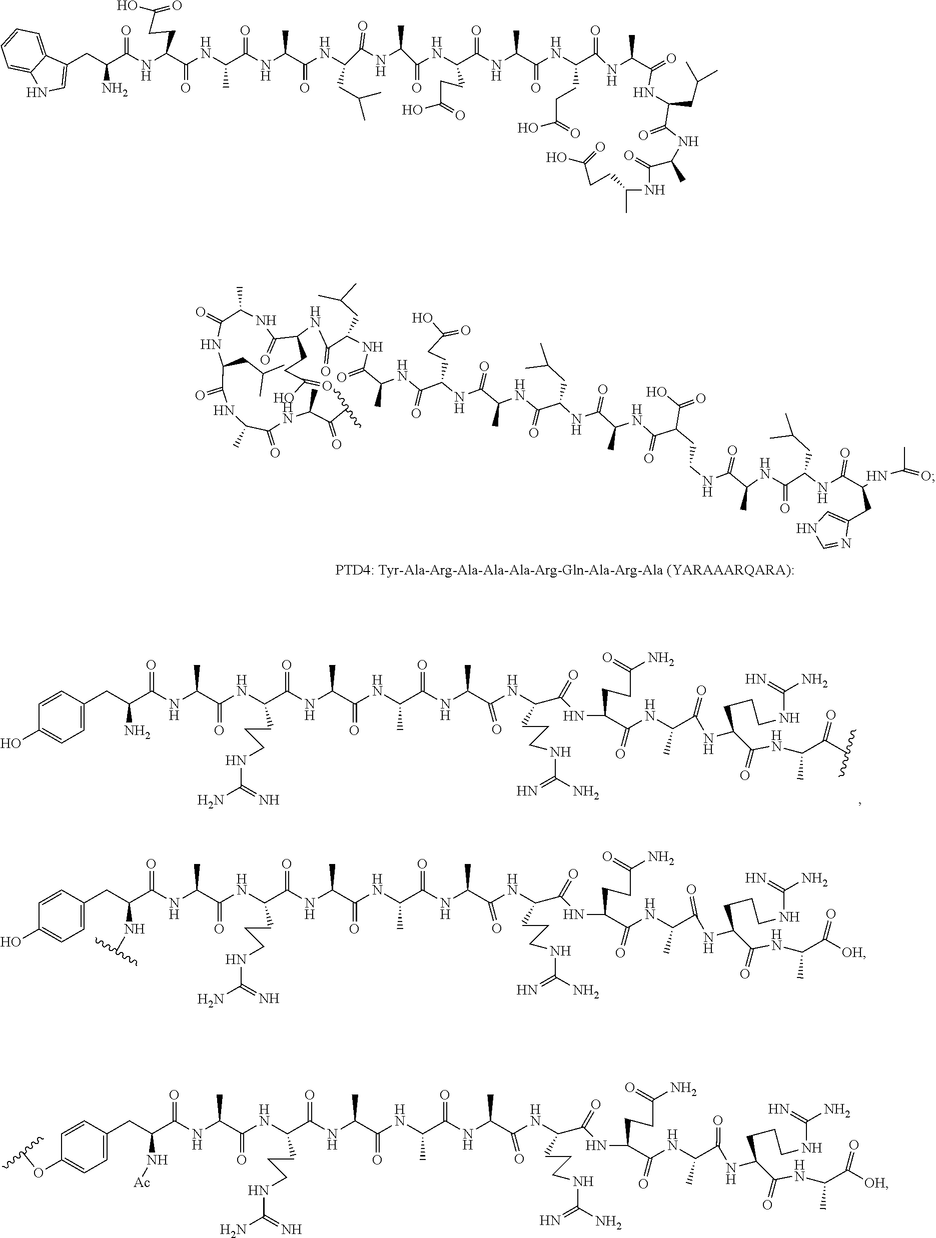 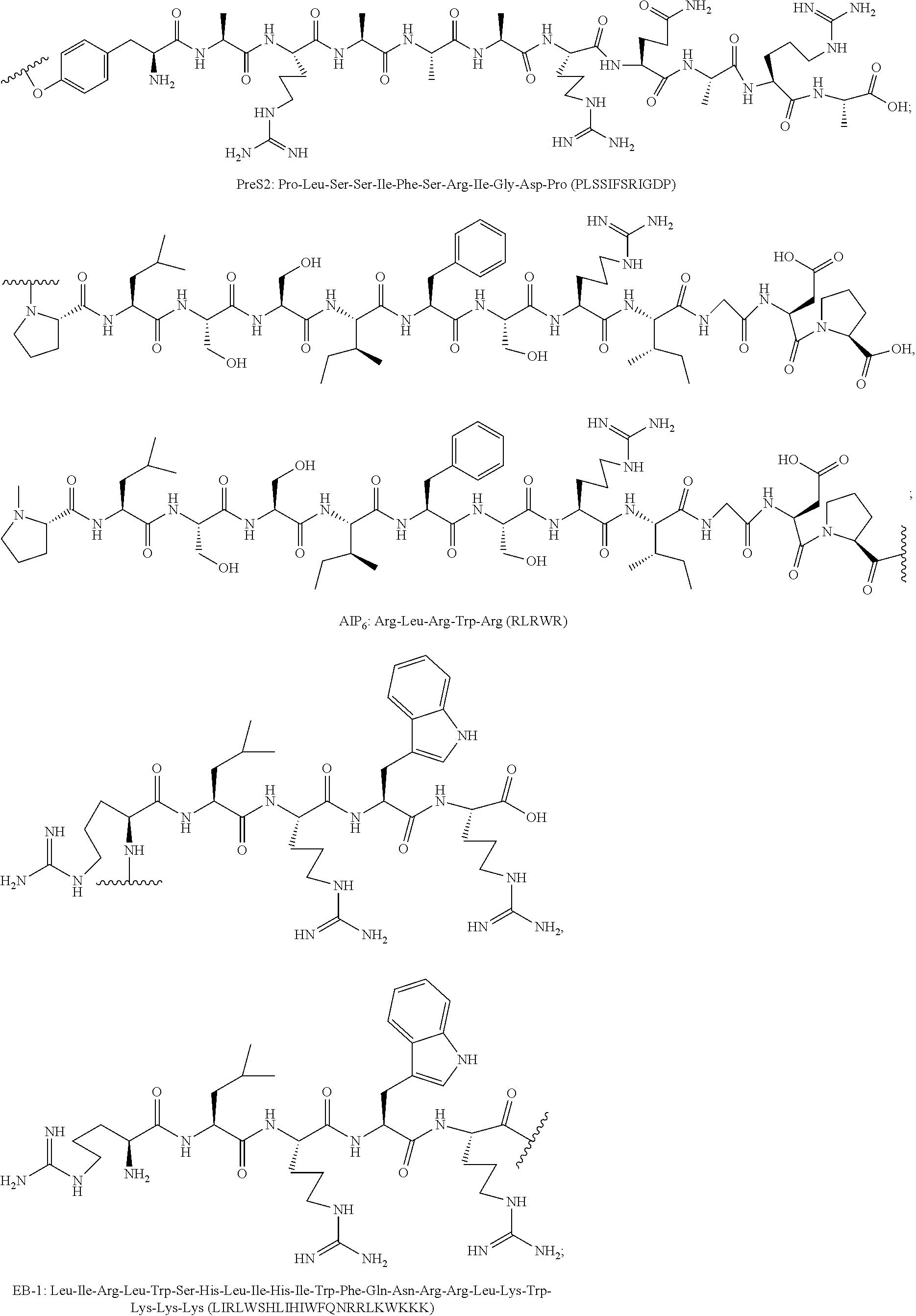 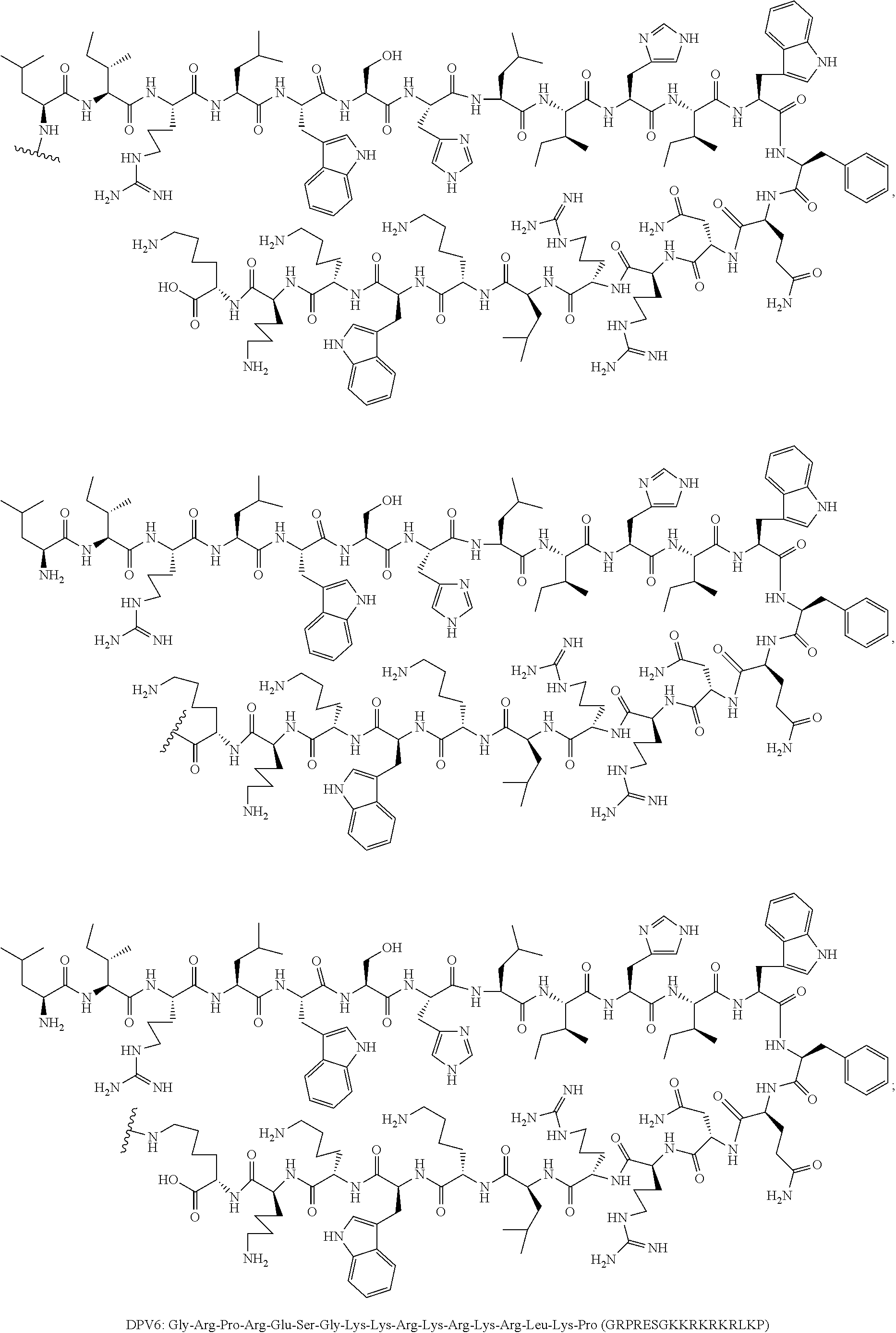 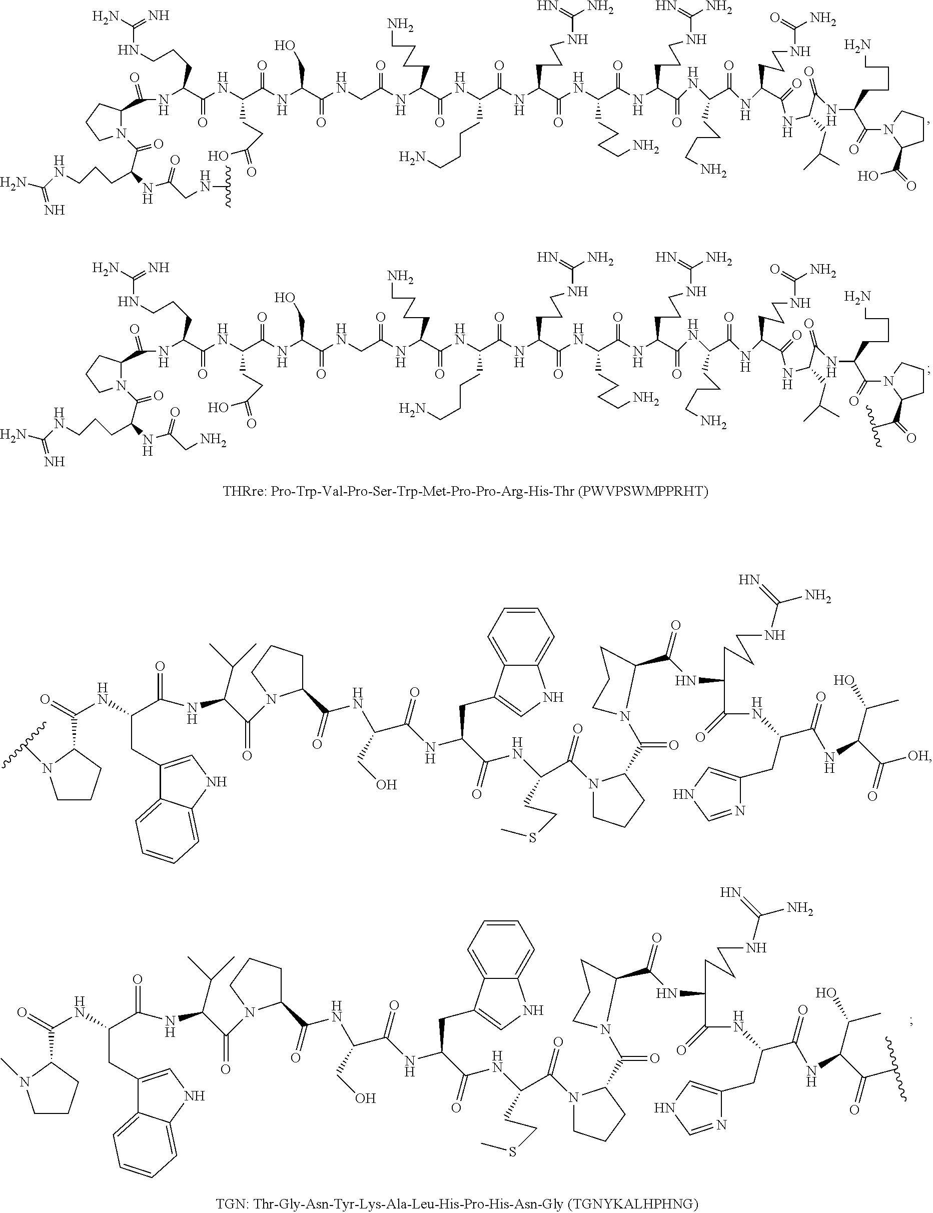 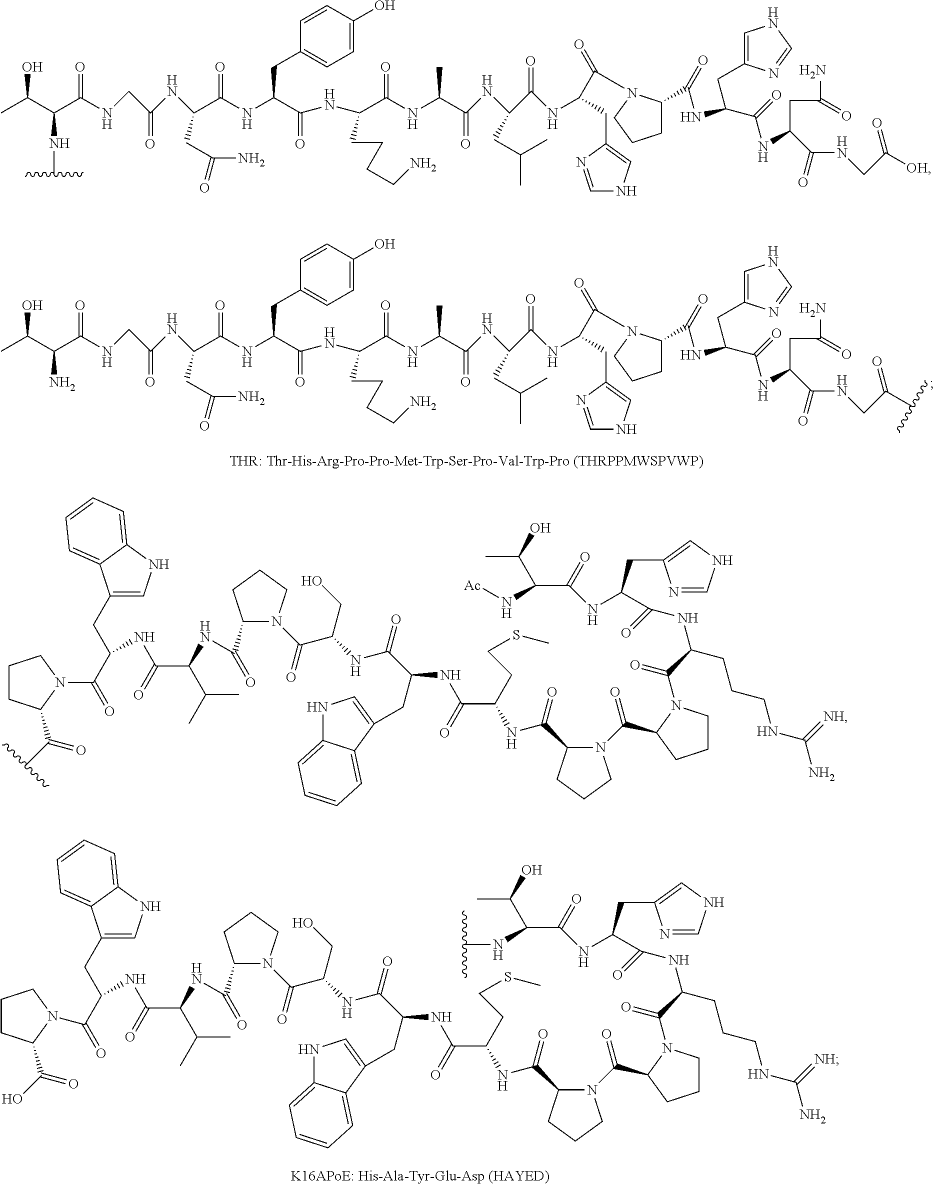 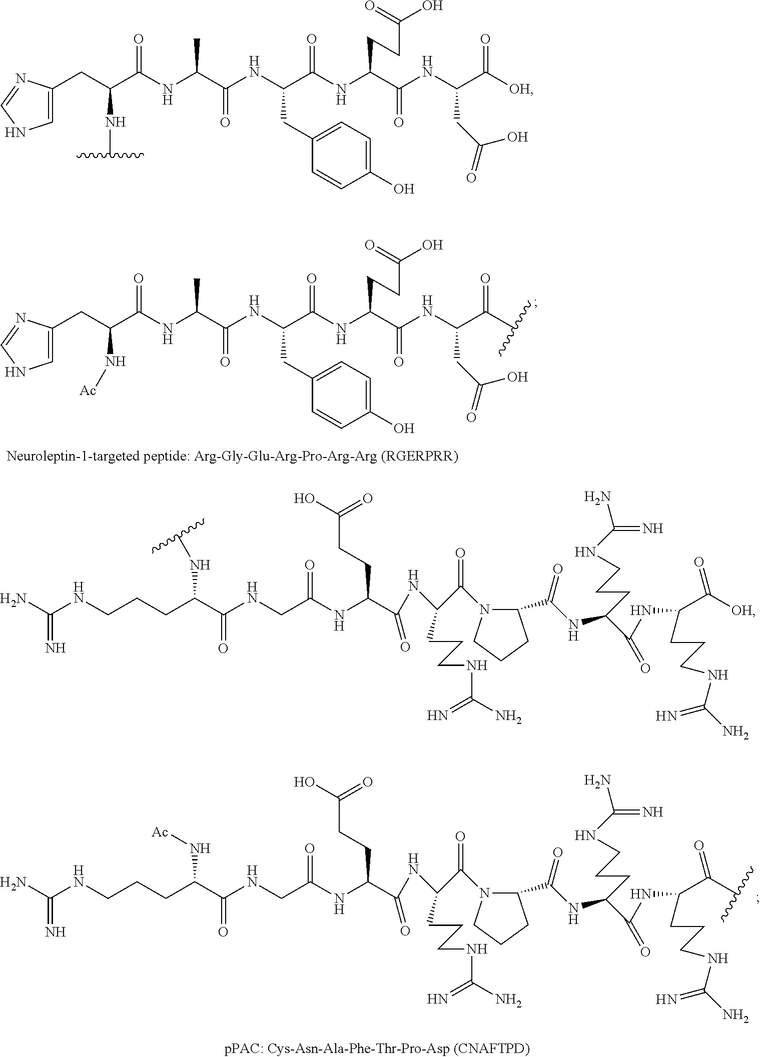 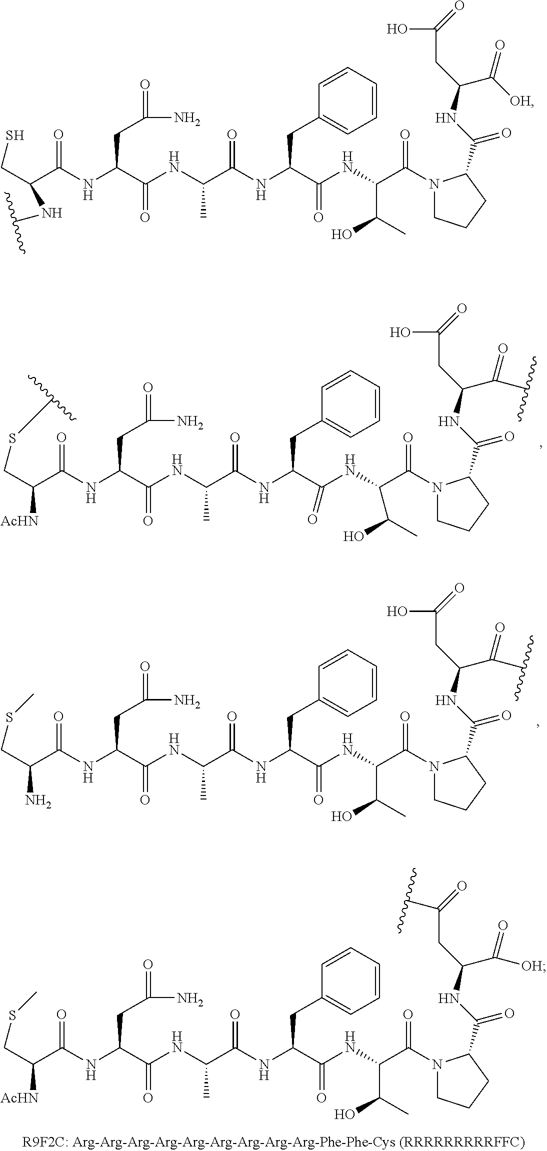 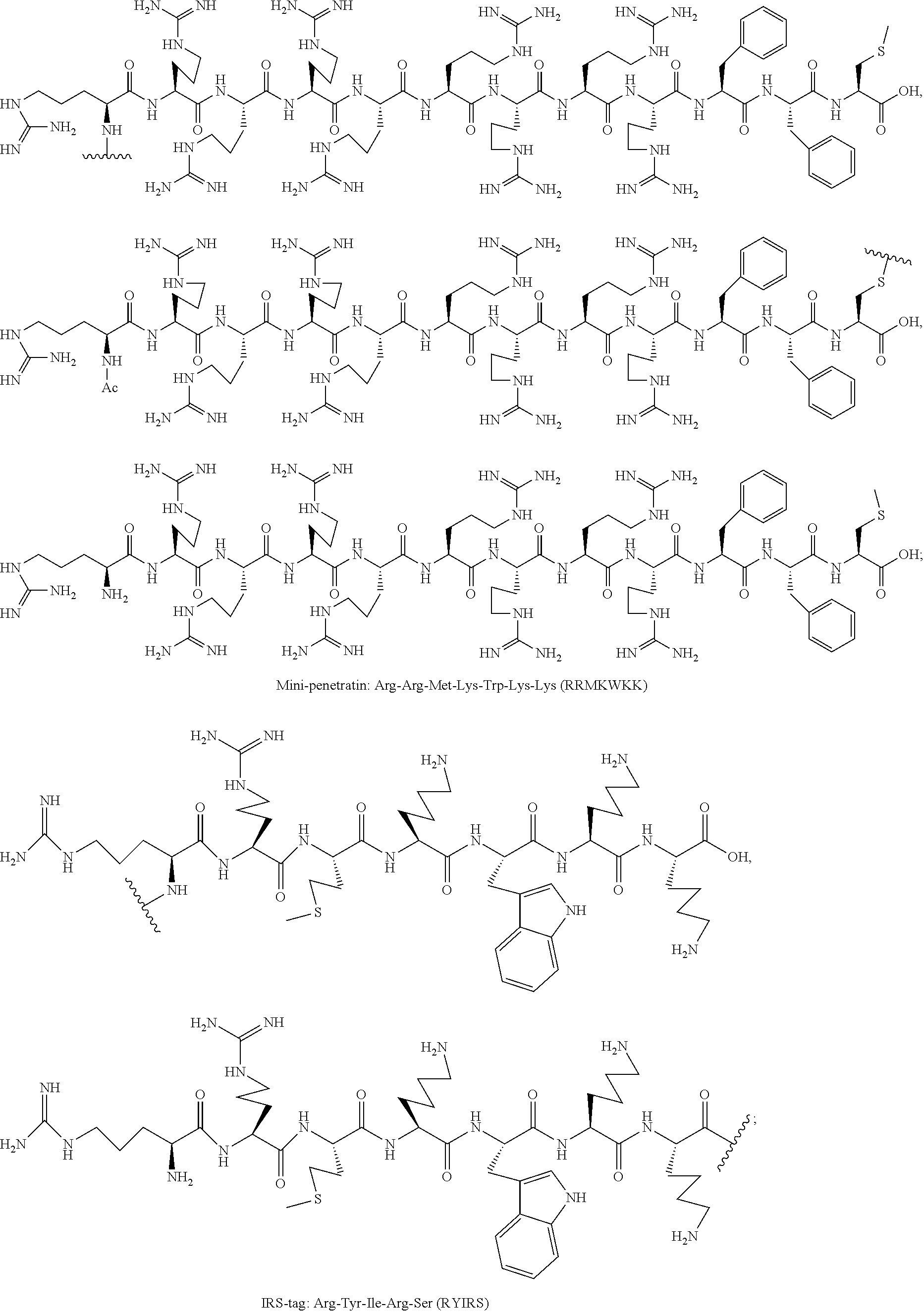 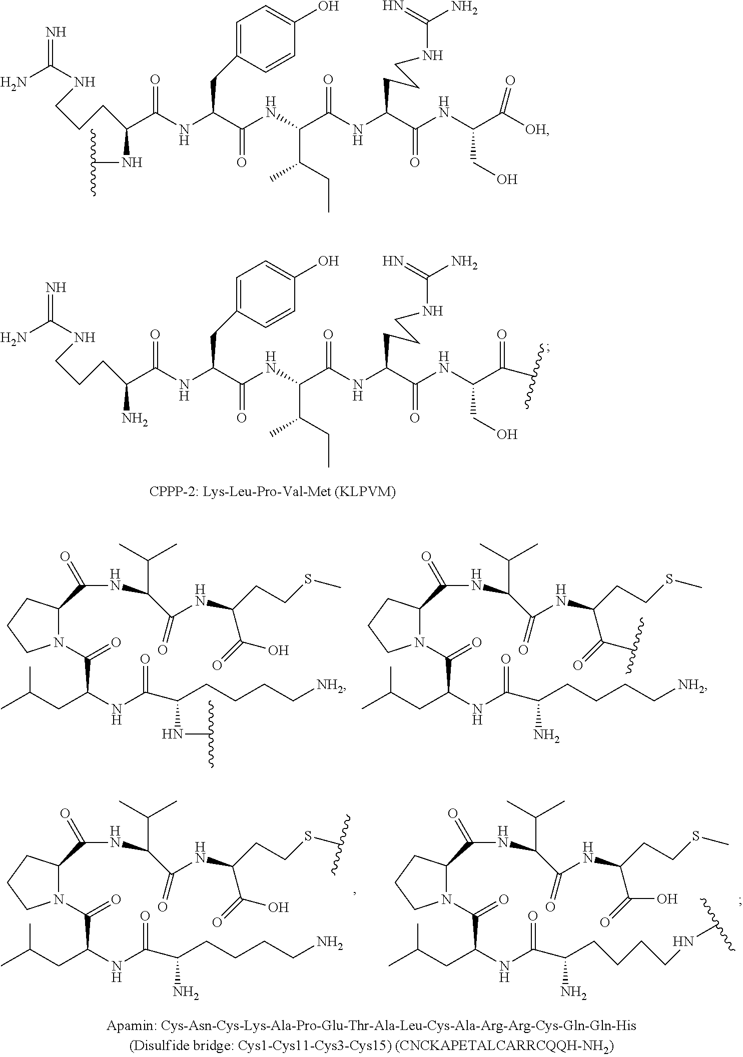 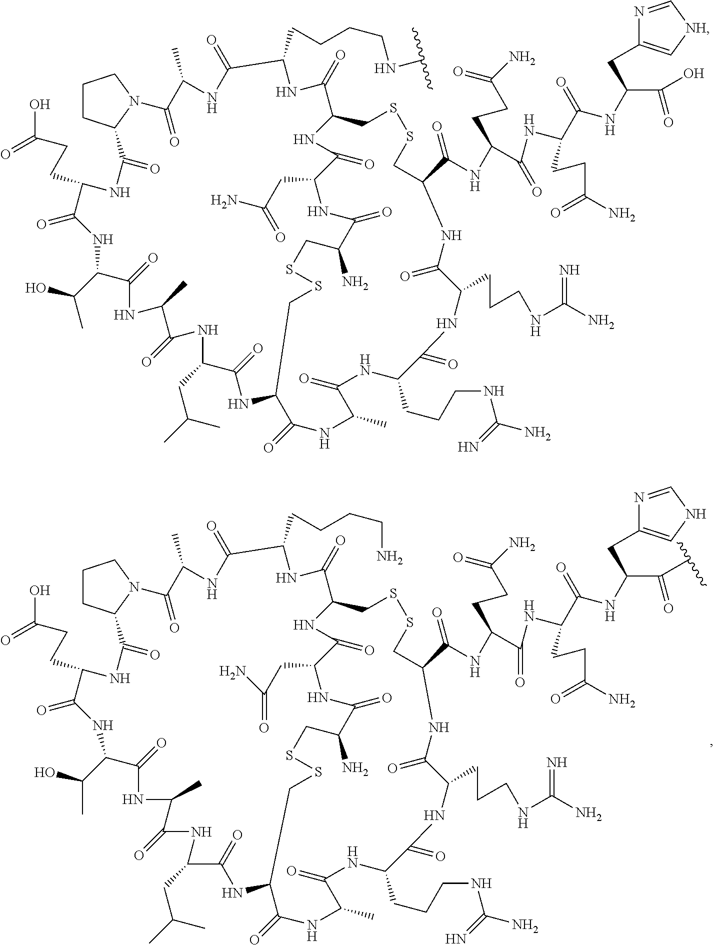 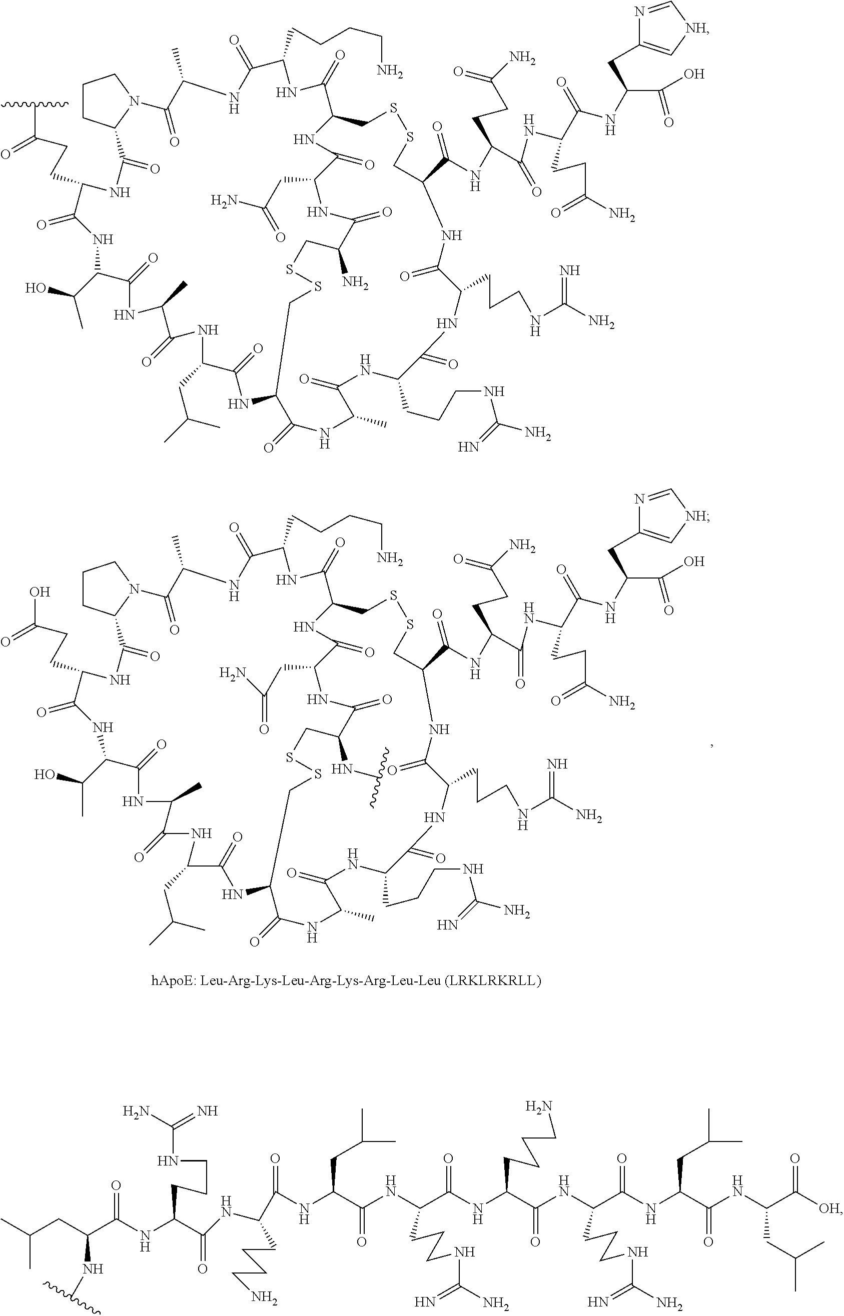 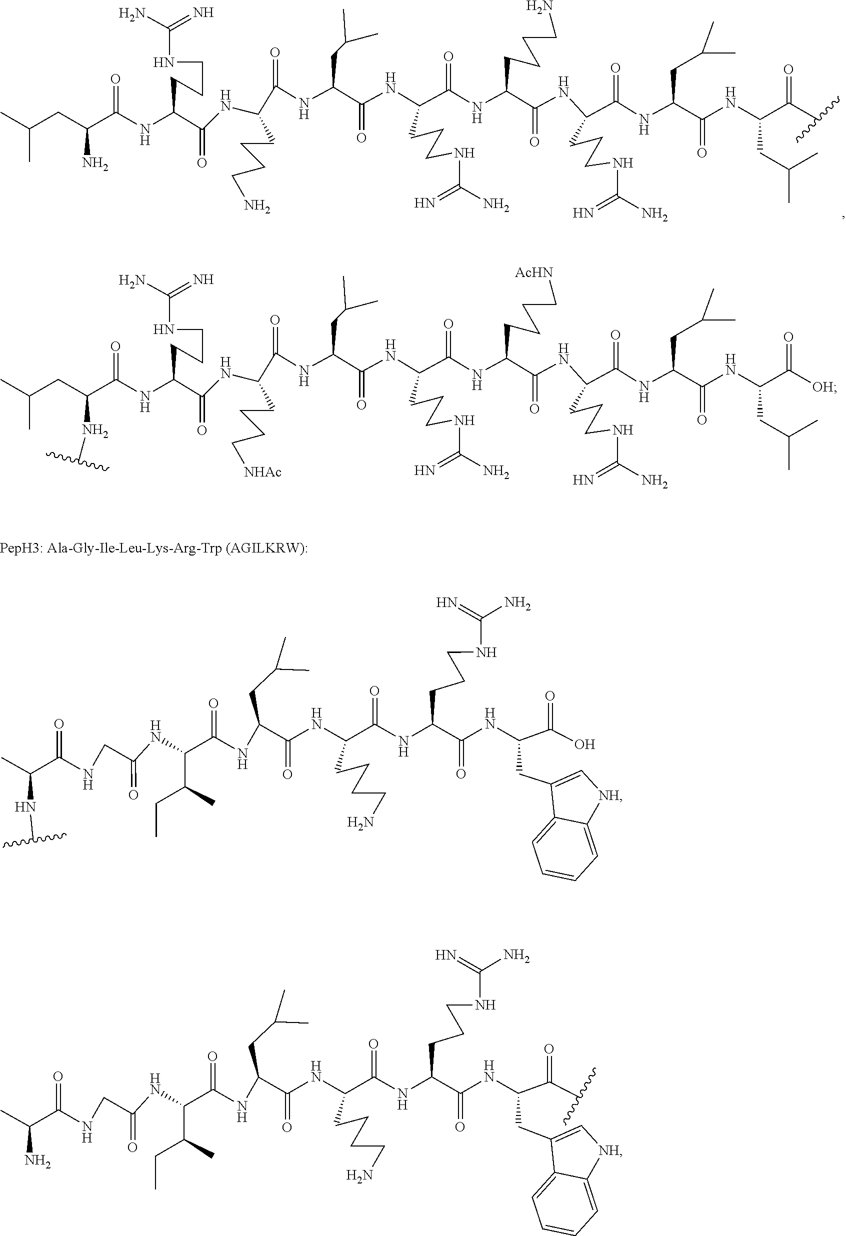 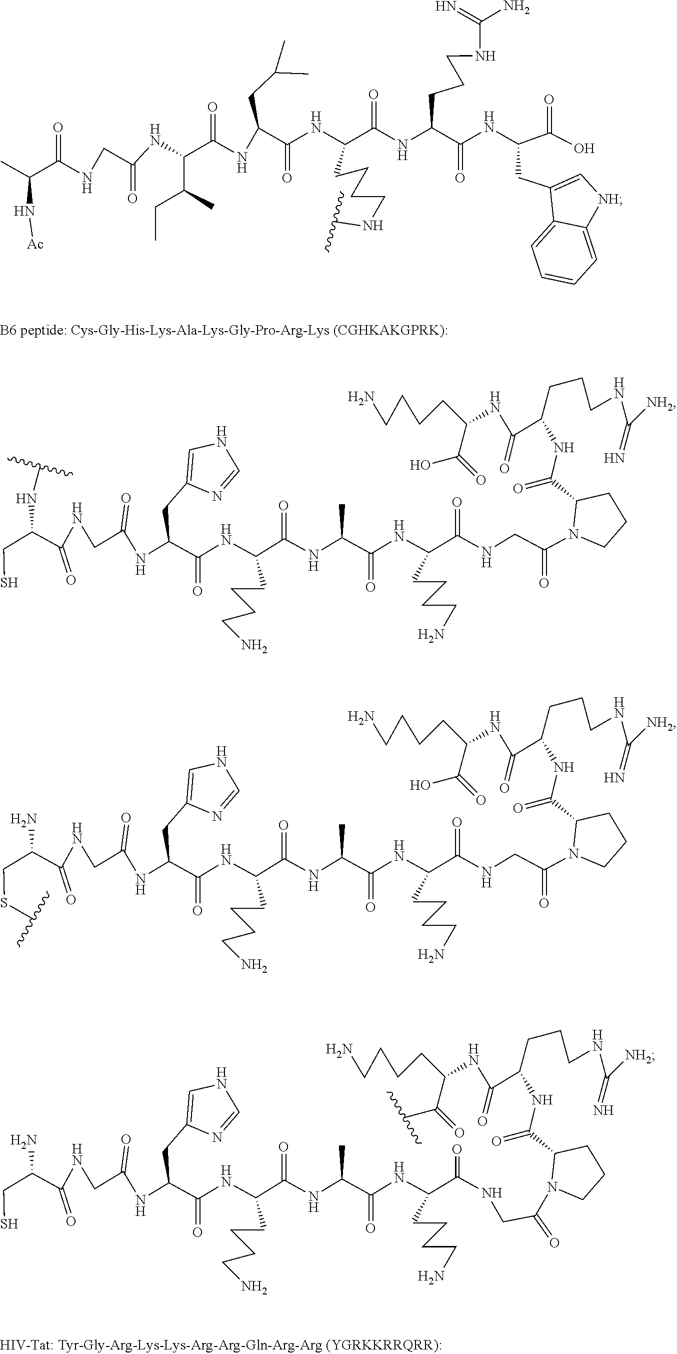 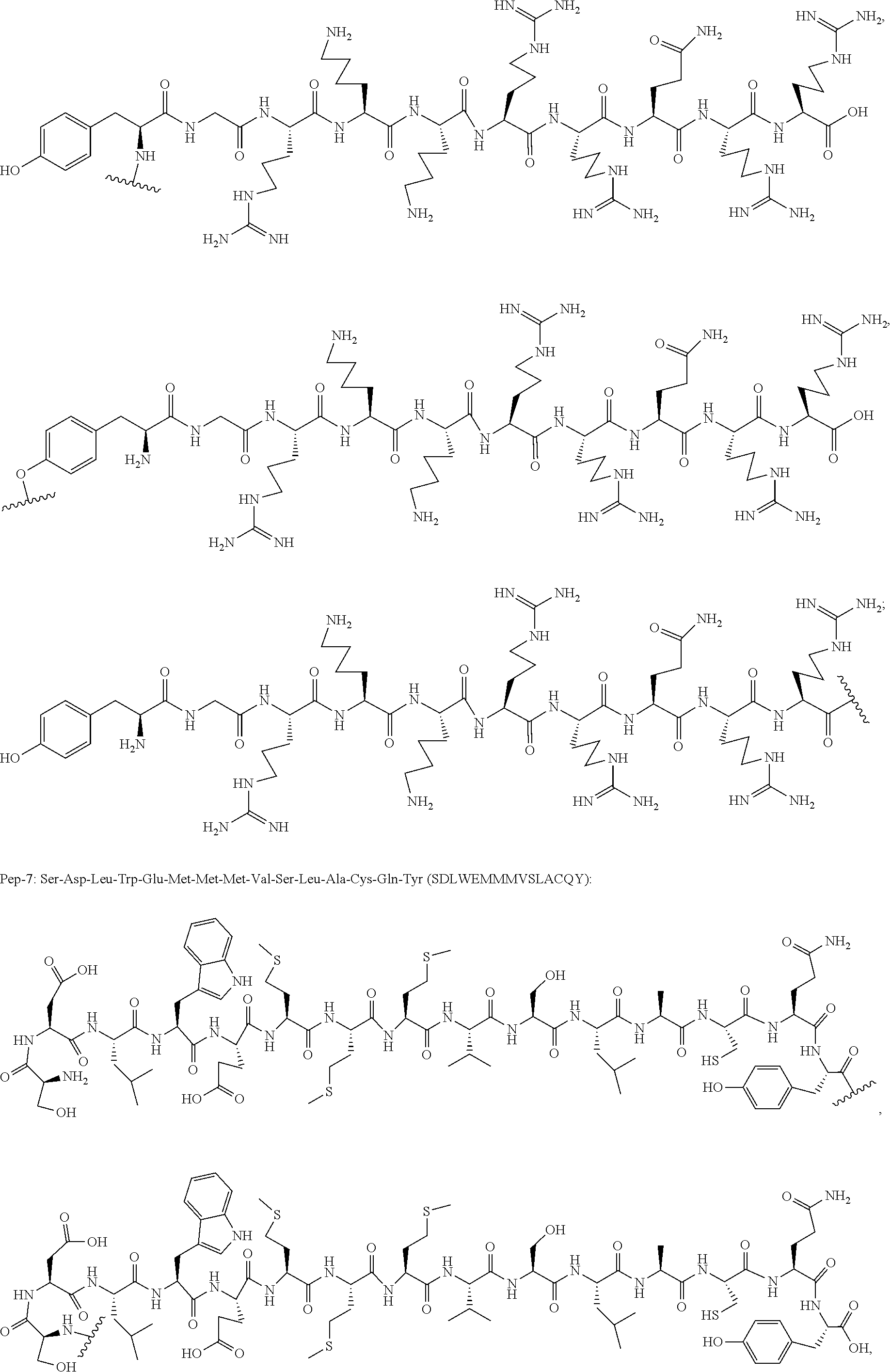 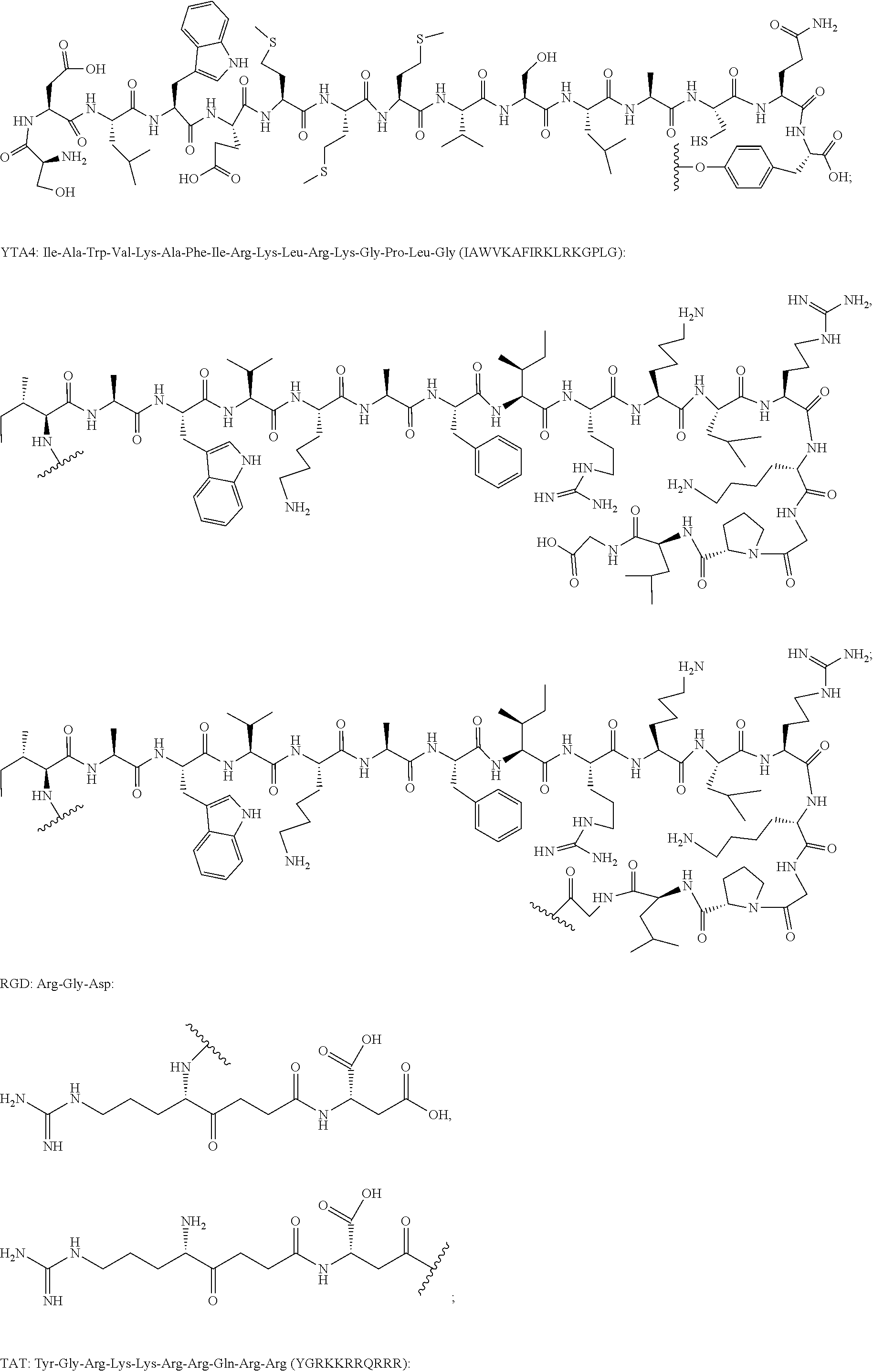 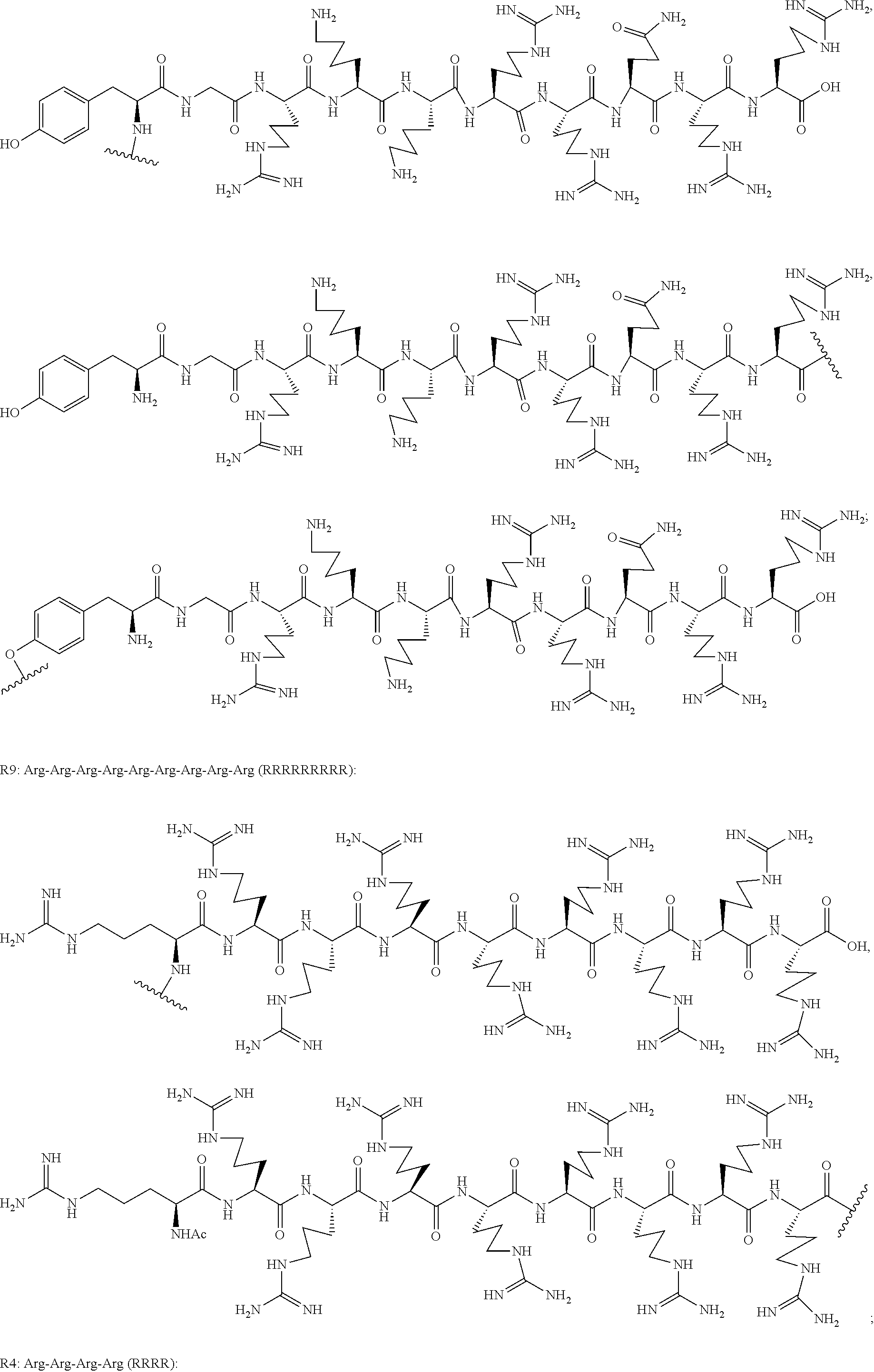 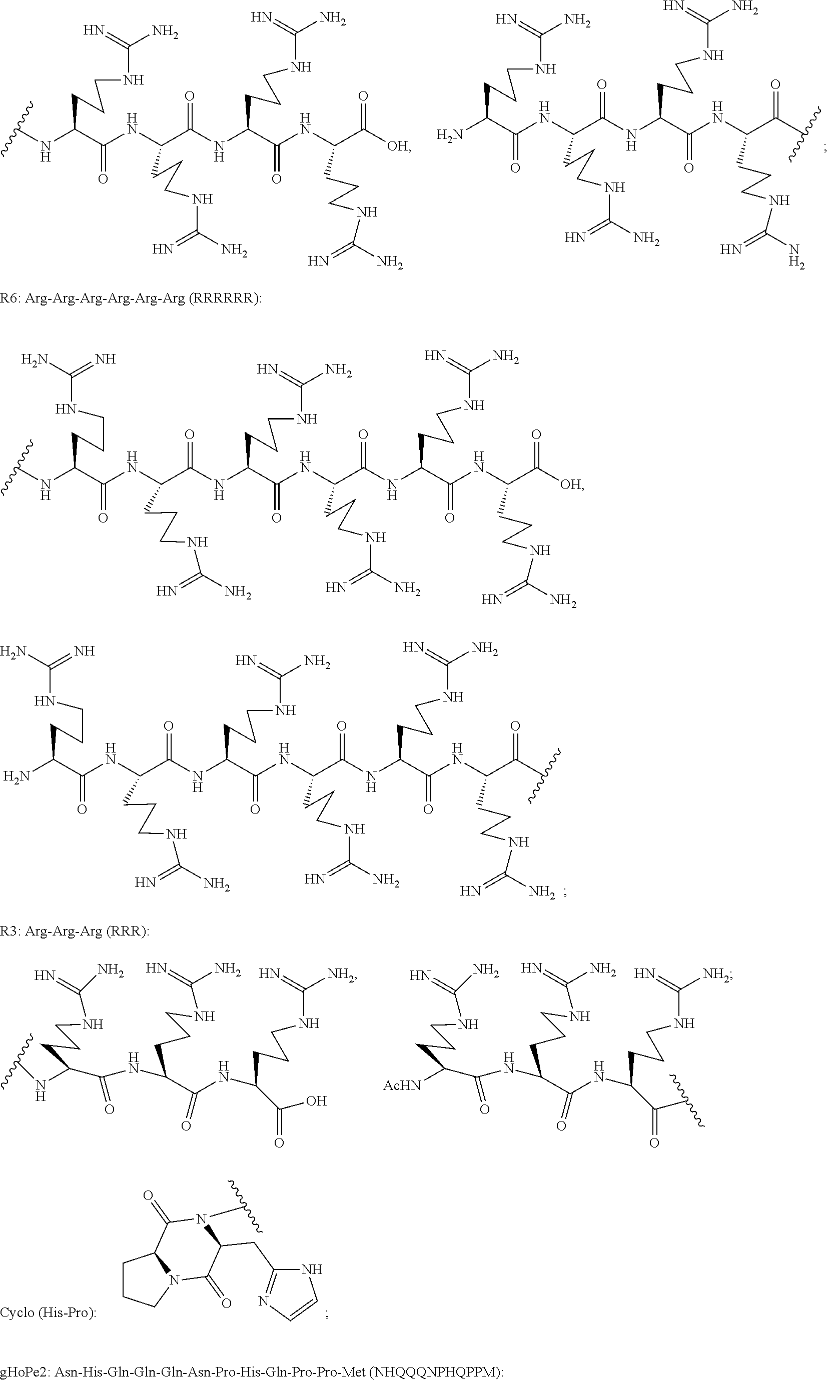 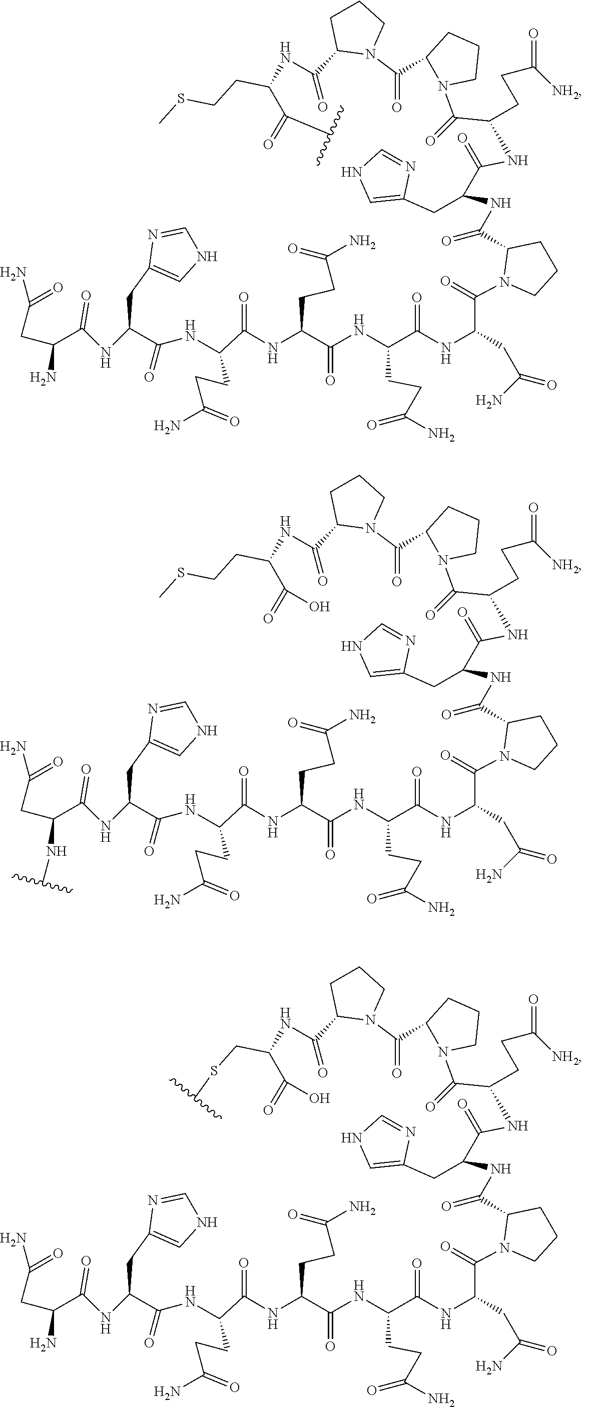 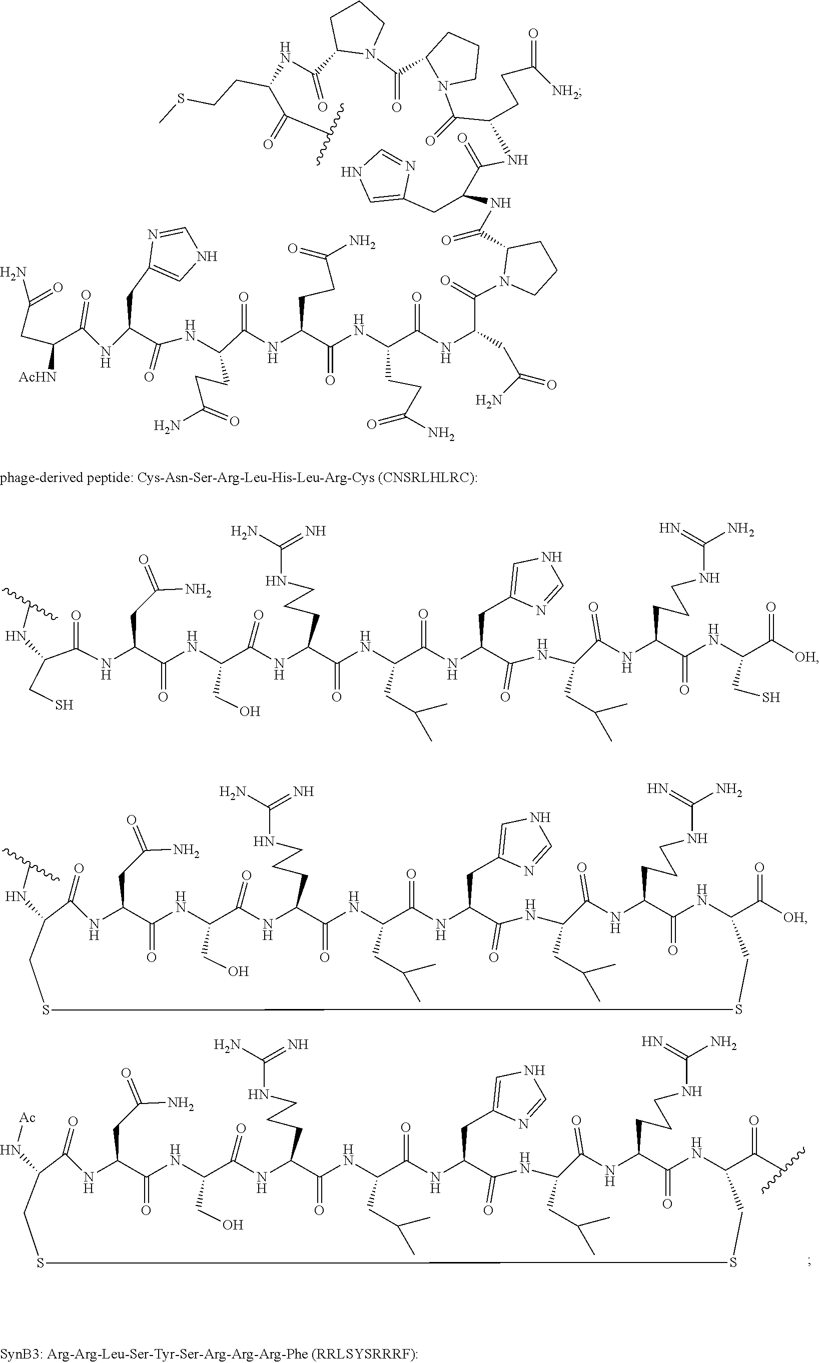 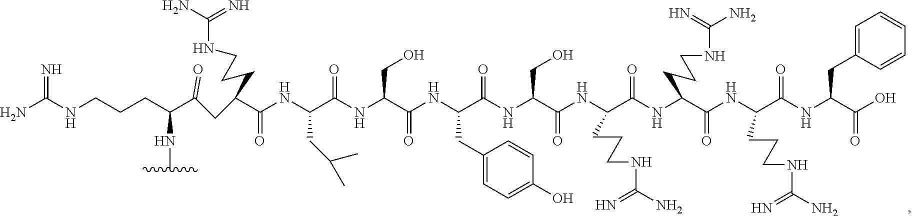 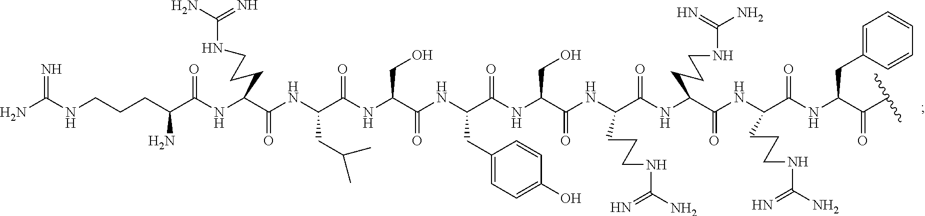 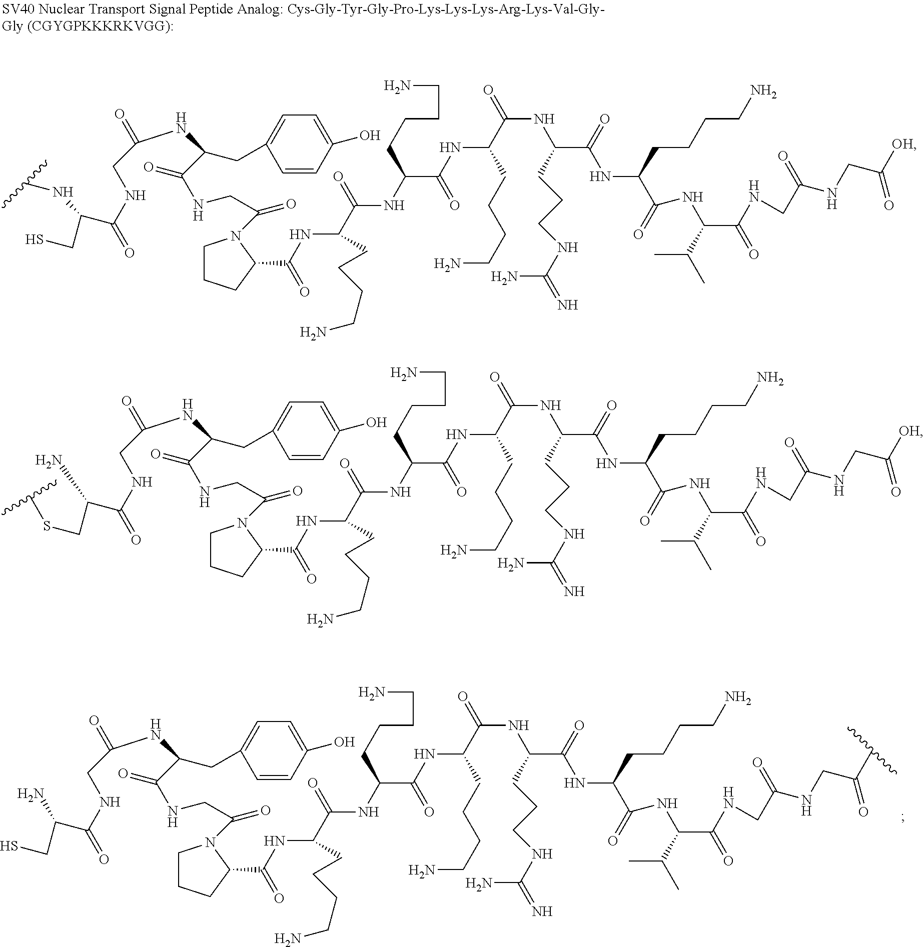 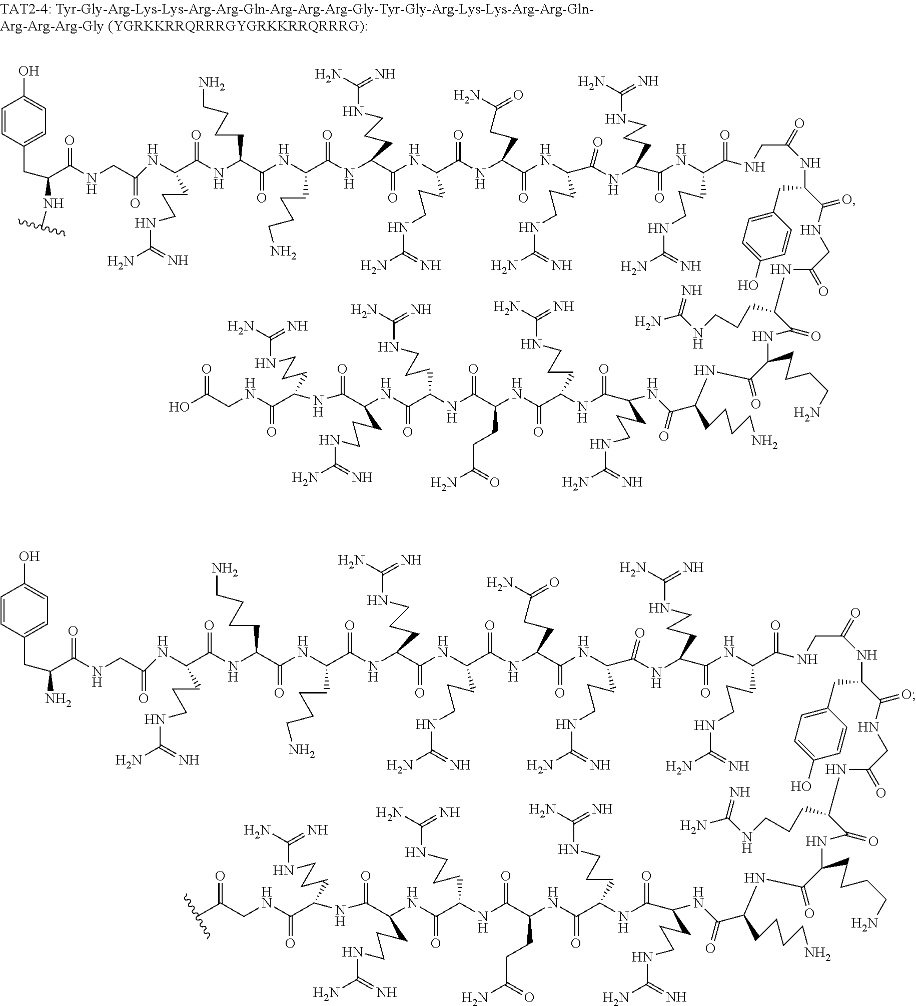 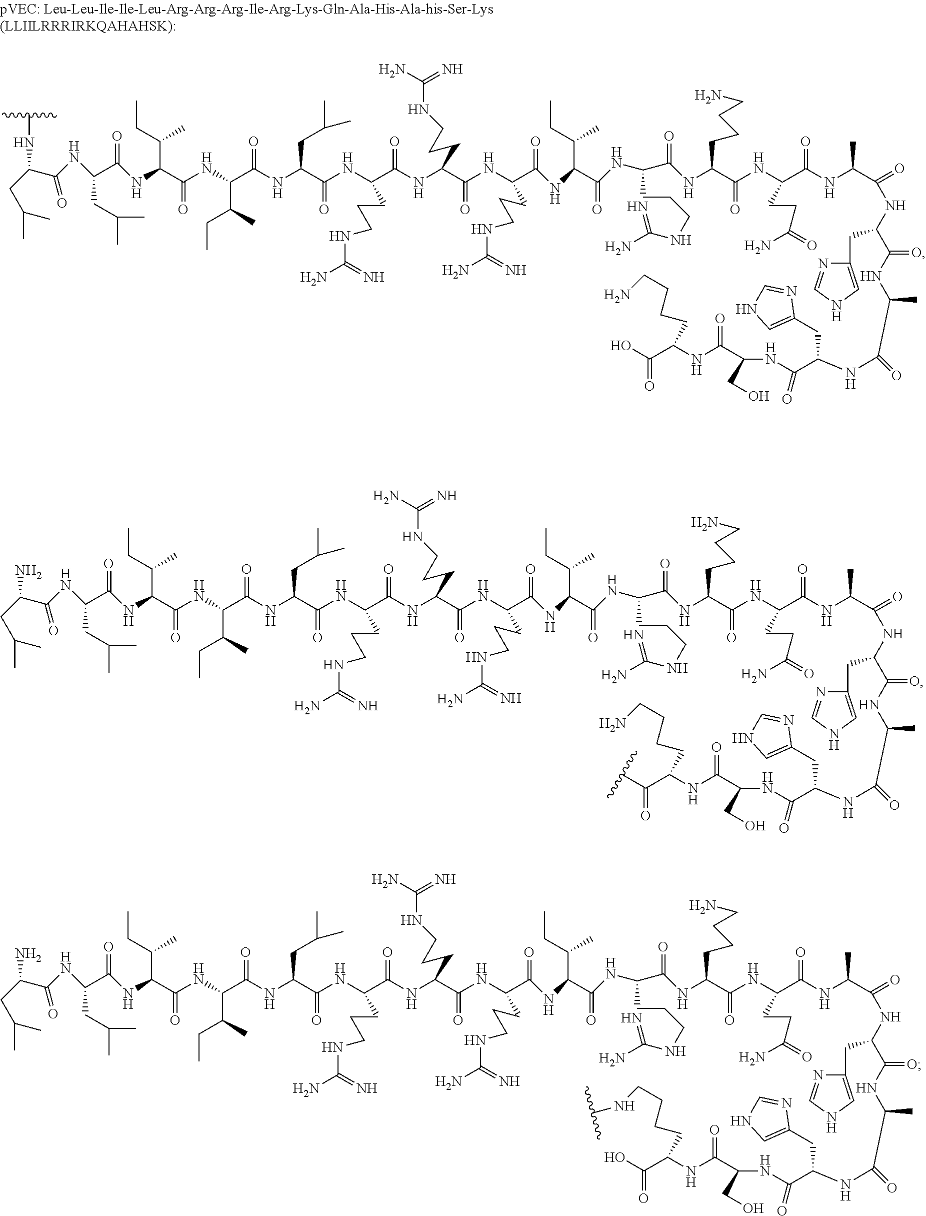 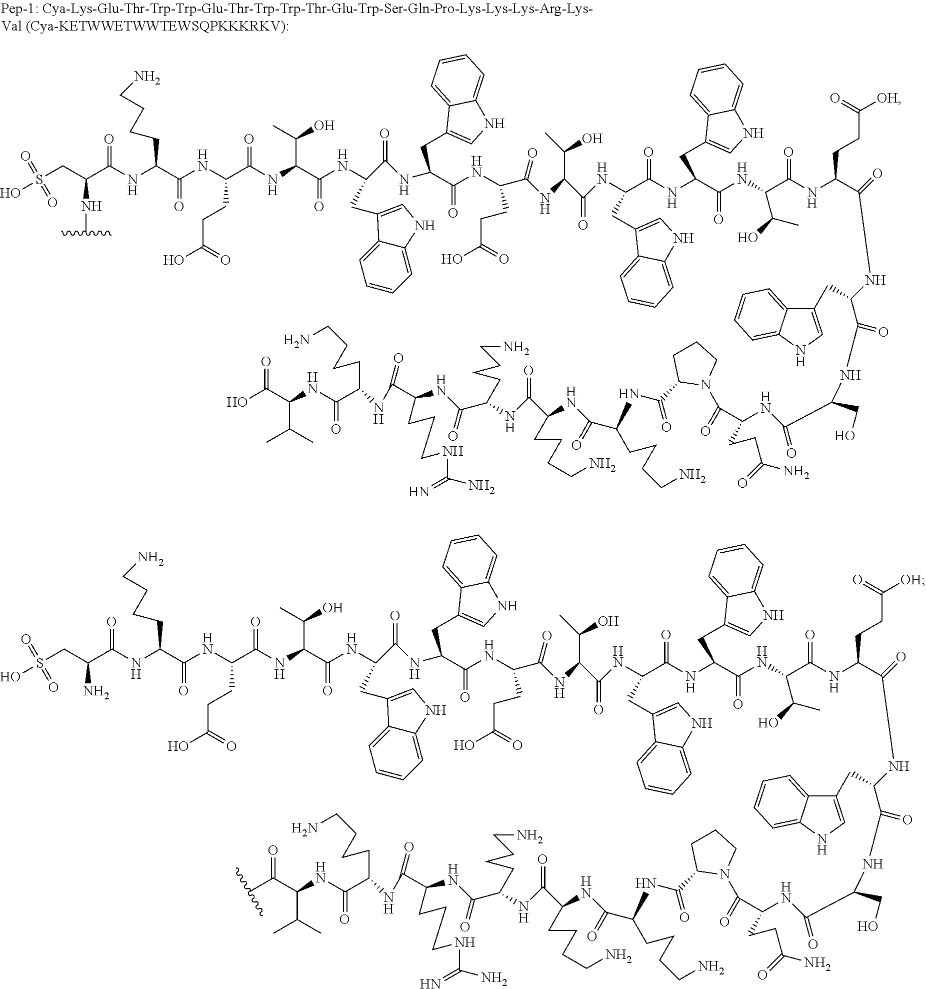 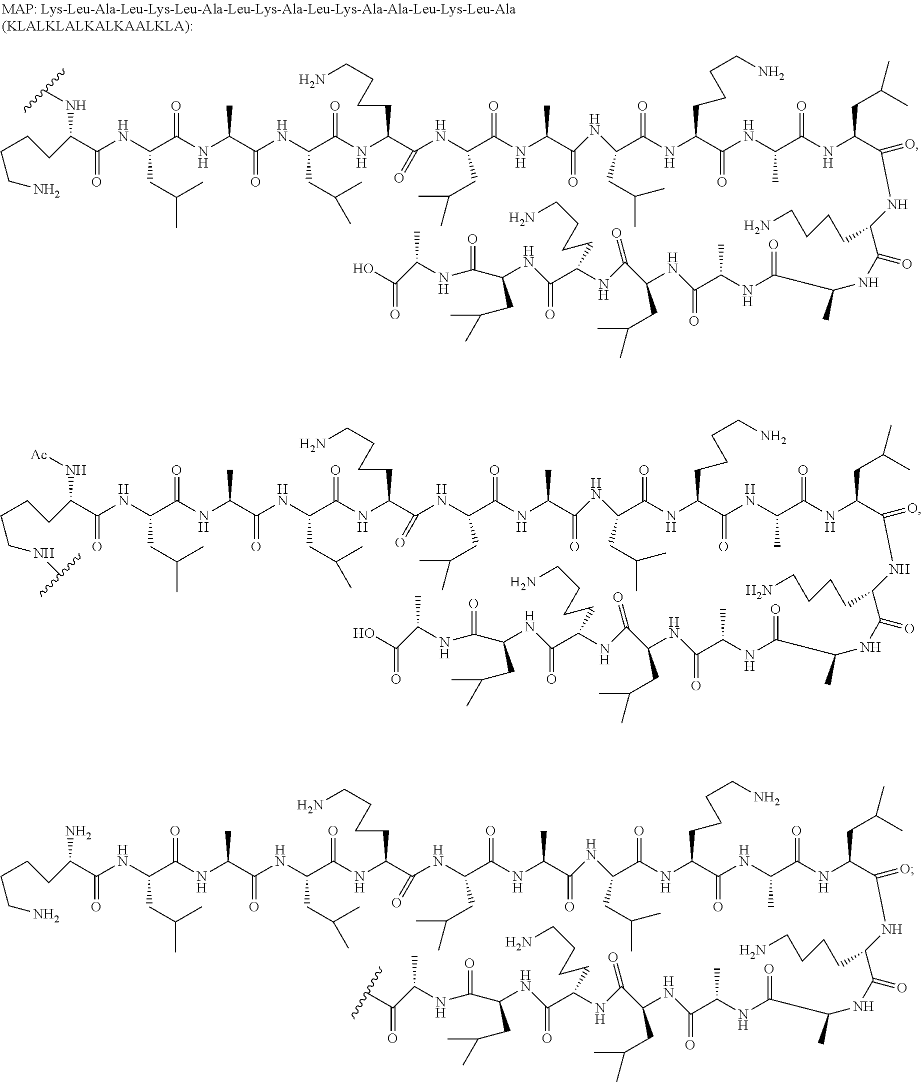 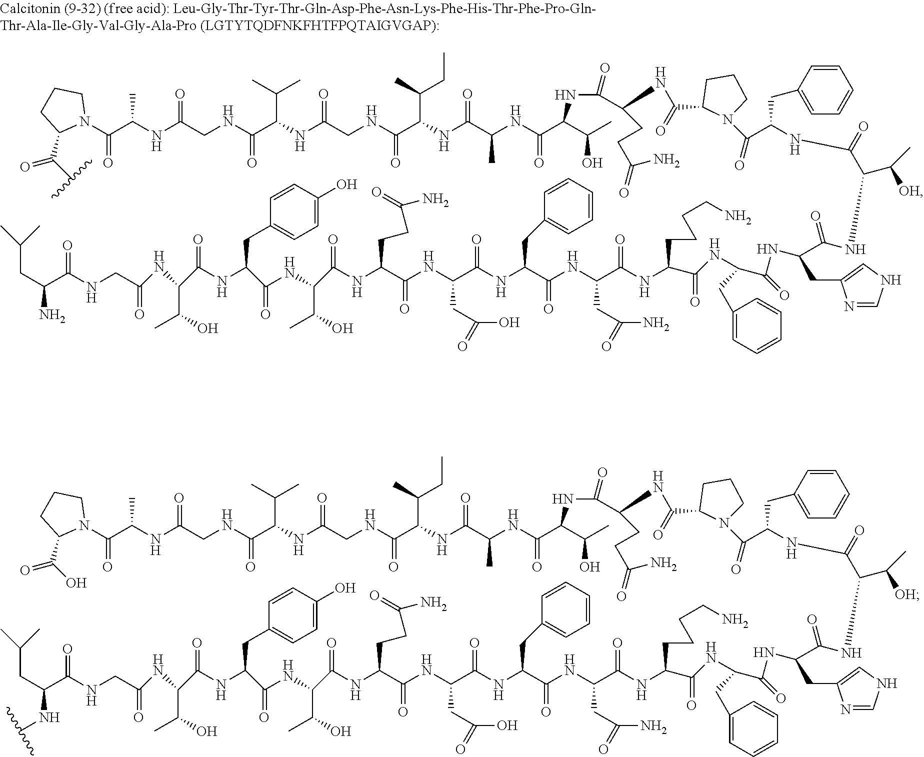 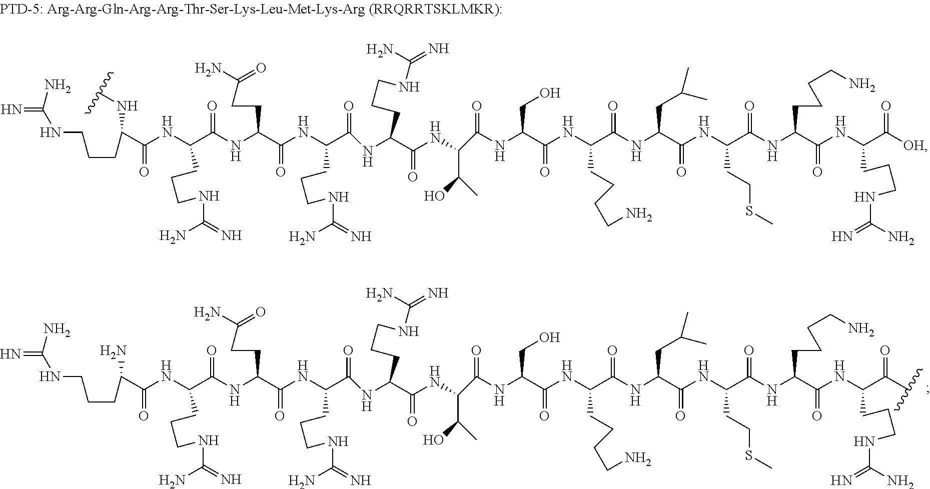 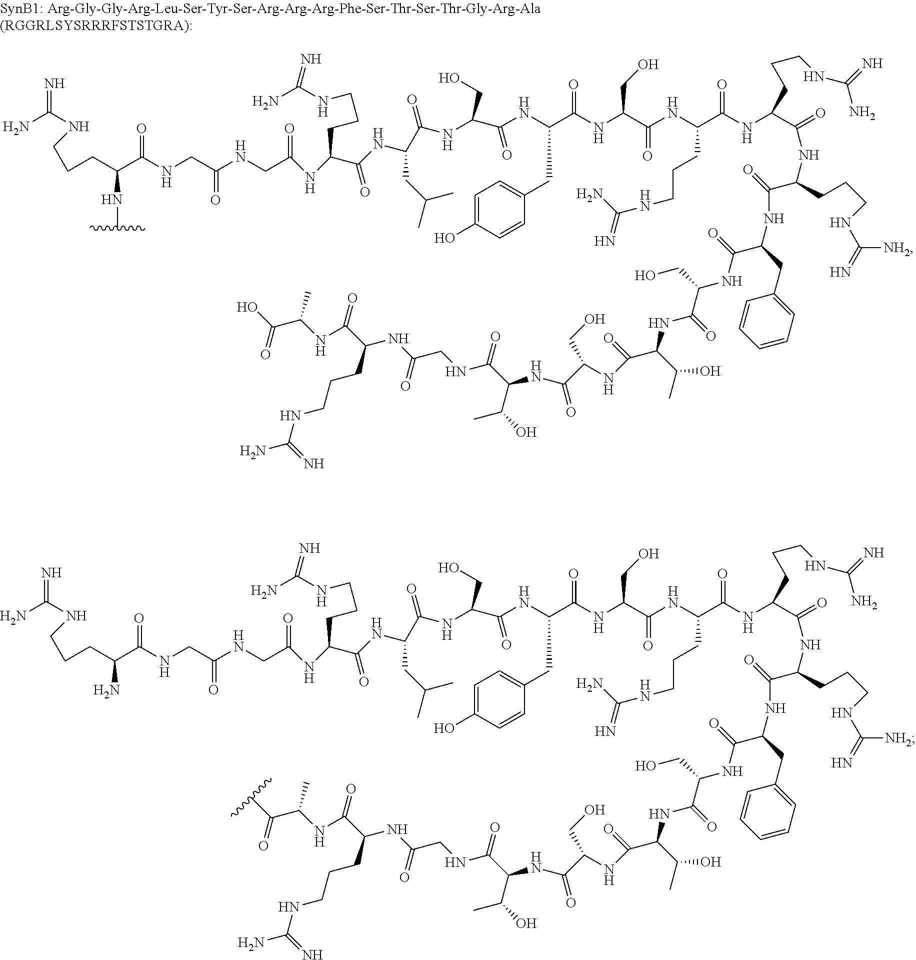 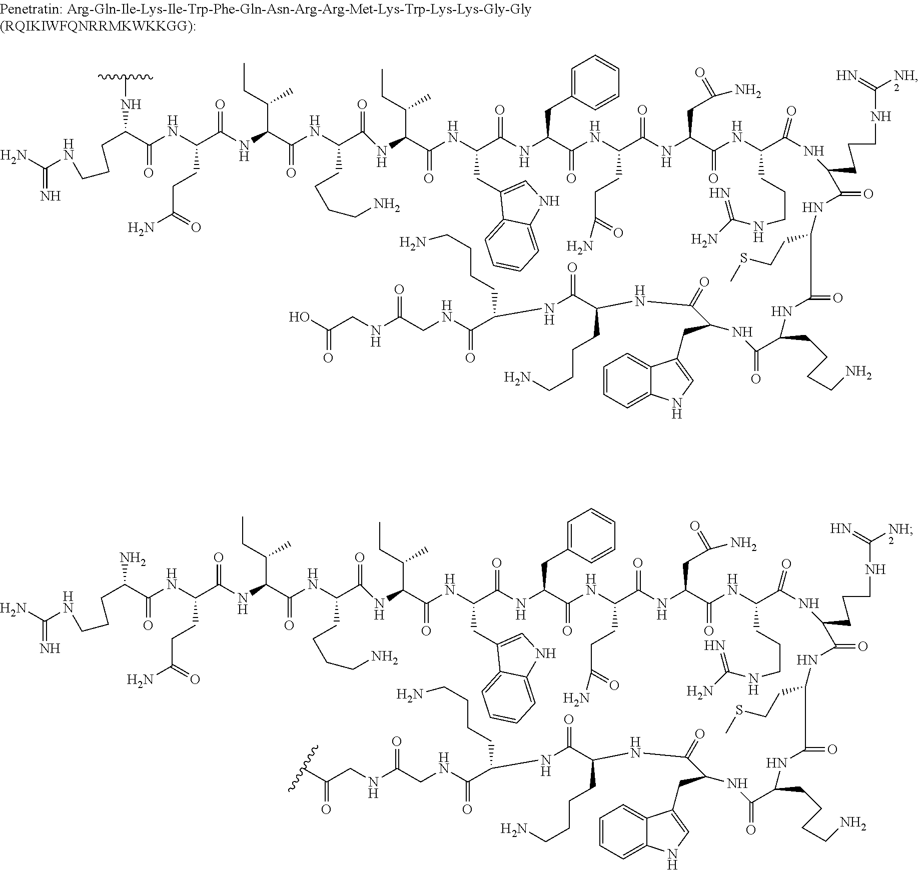 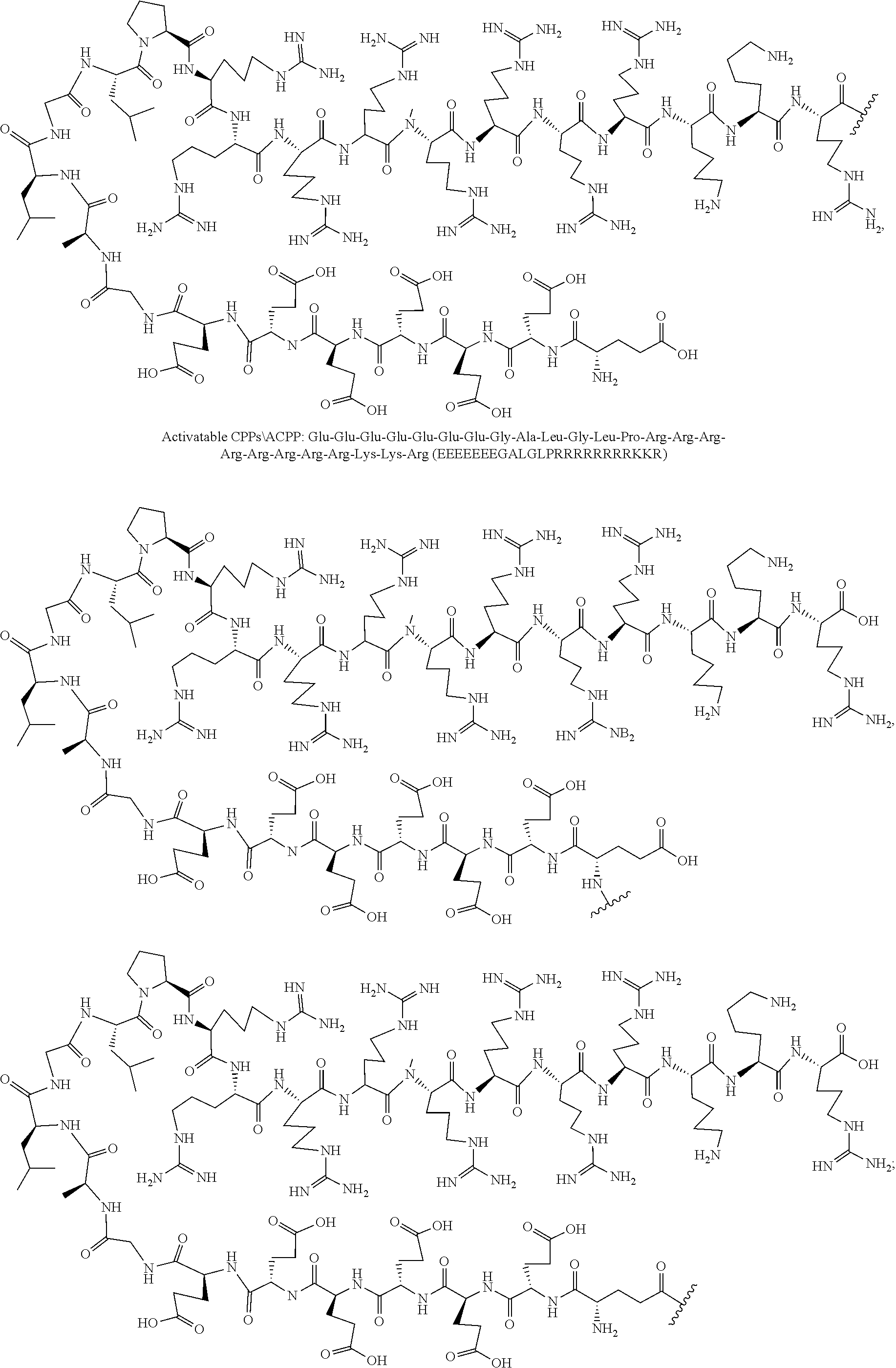 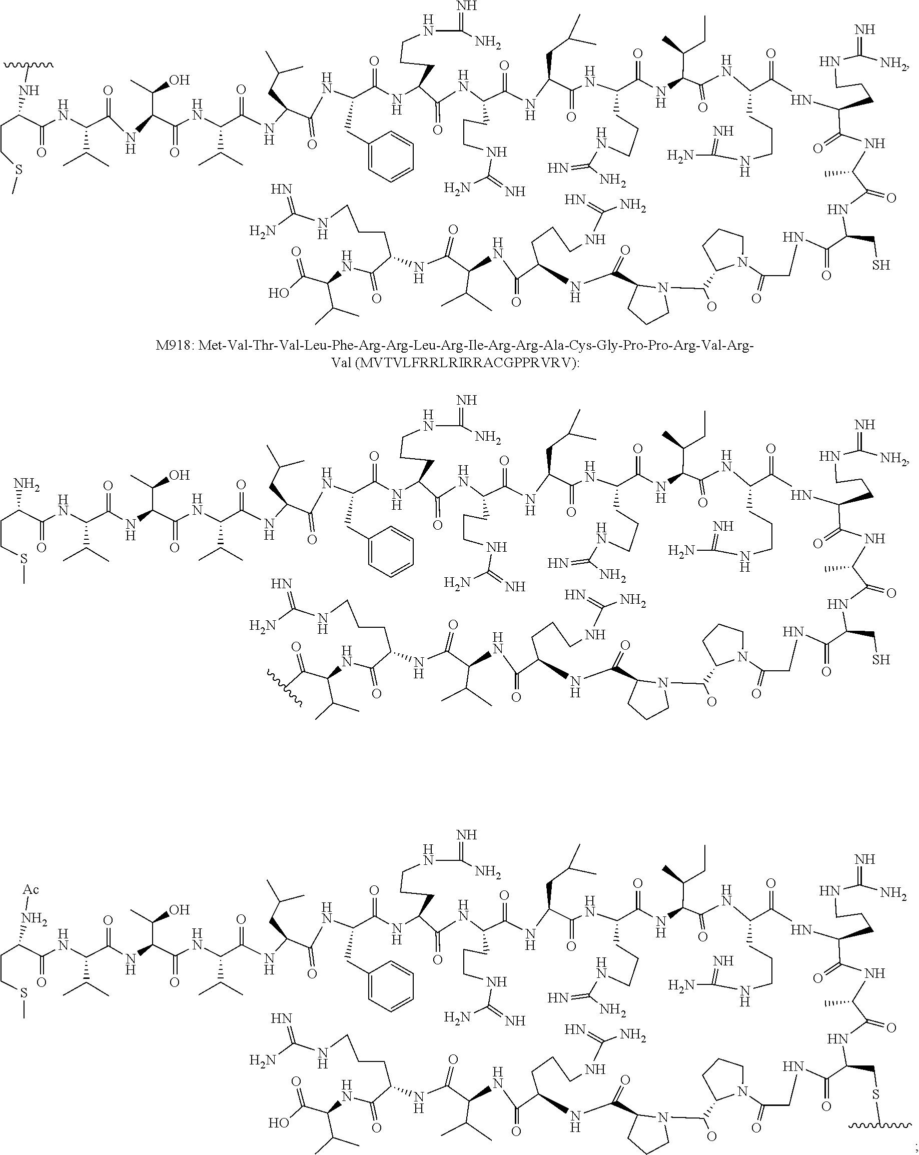 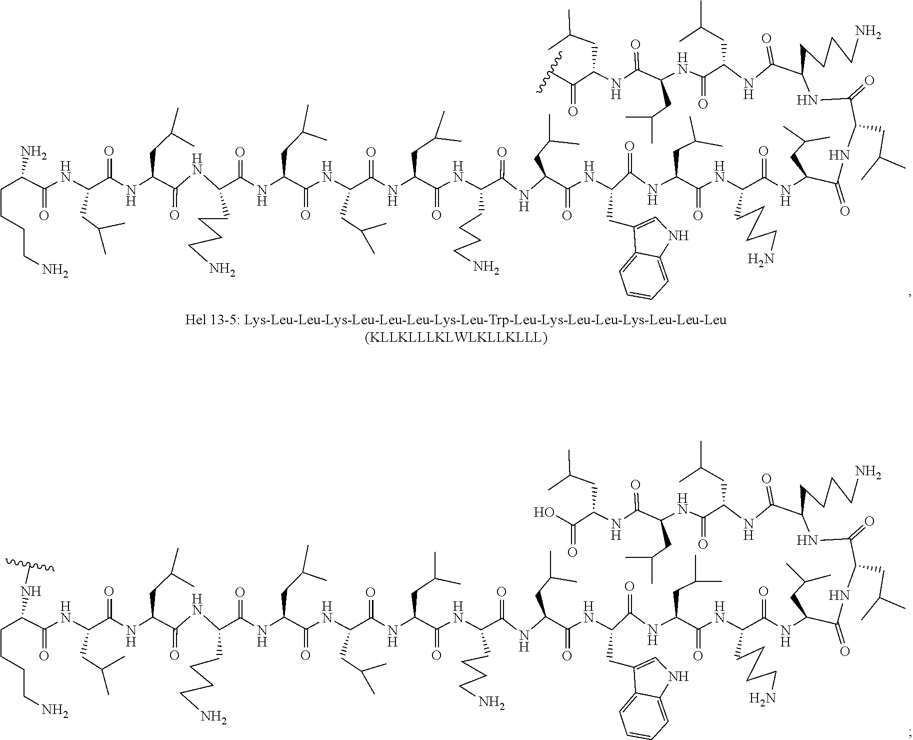 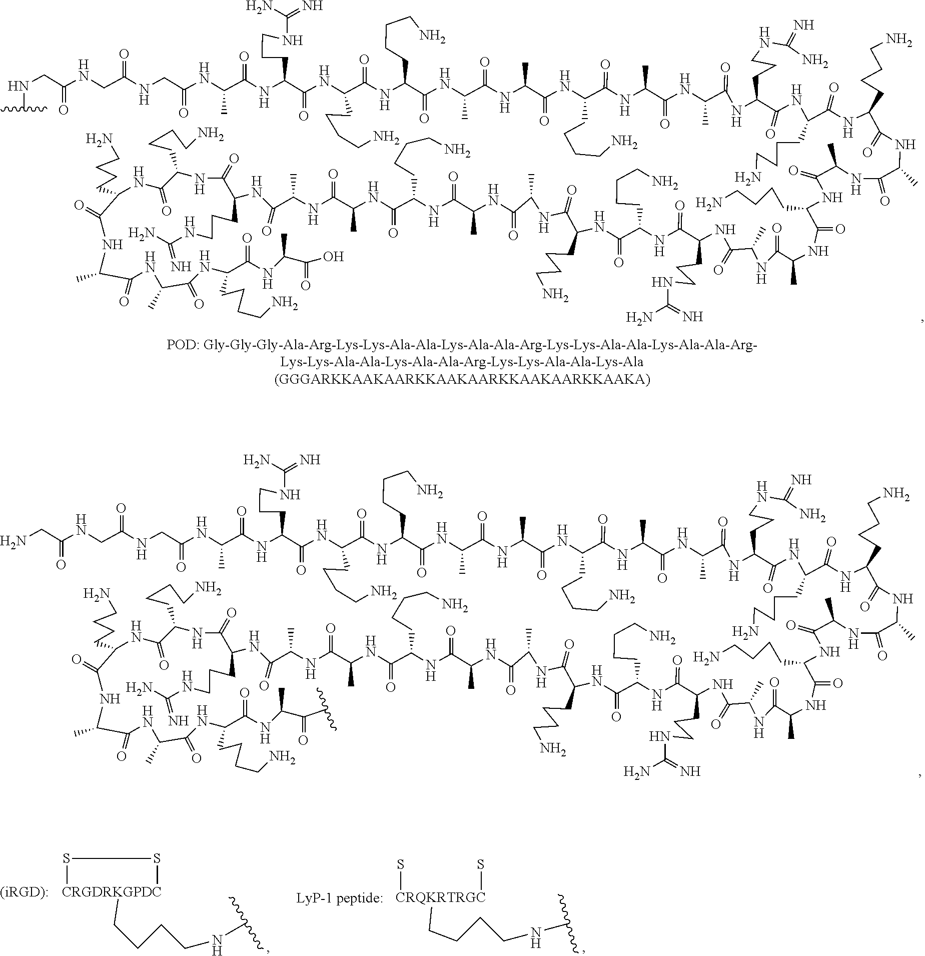
wherein [symbol] is the site linked to Ld₁, Ld₂, Ld₃, Ld₄, Ld₅, or Ld₆; Ra is Ar, which is preferably selected from 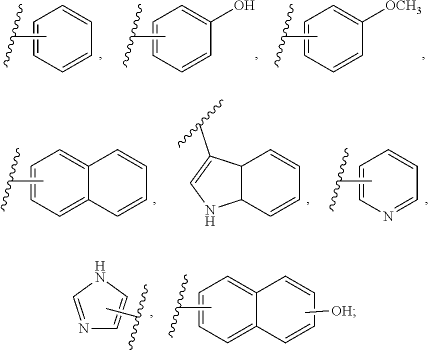
Rb is OH, COOH, COOCH₃, CH₃OH, CH₃NH₂, CONH₂;
(2). the nucleoside antimetabolite/analog:

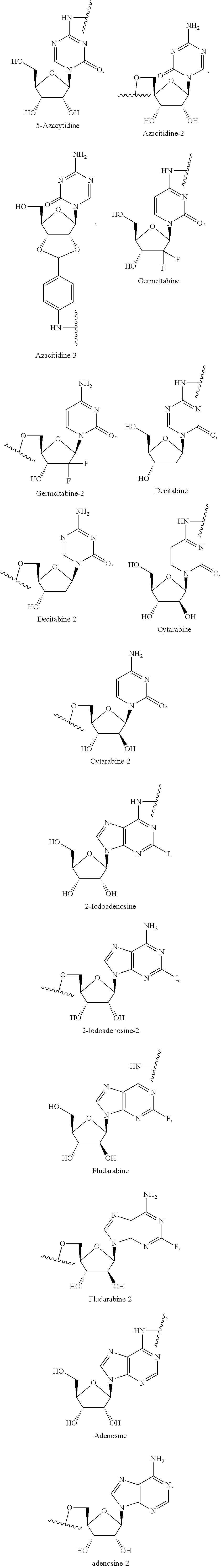

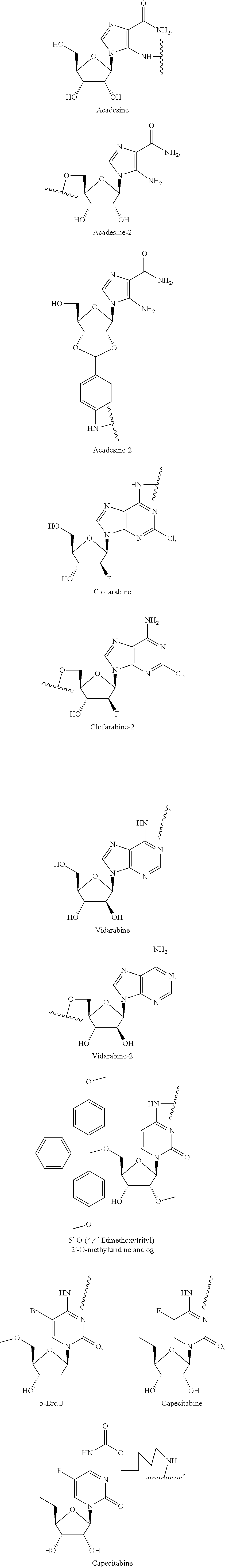

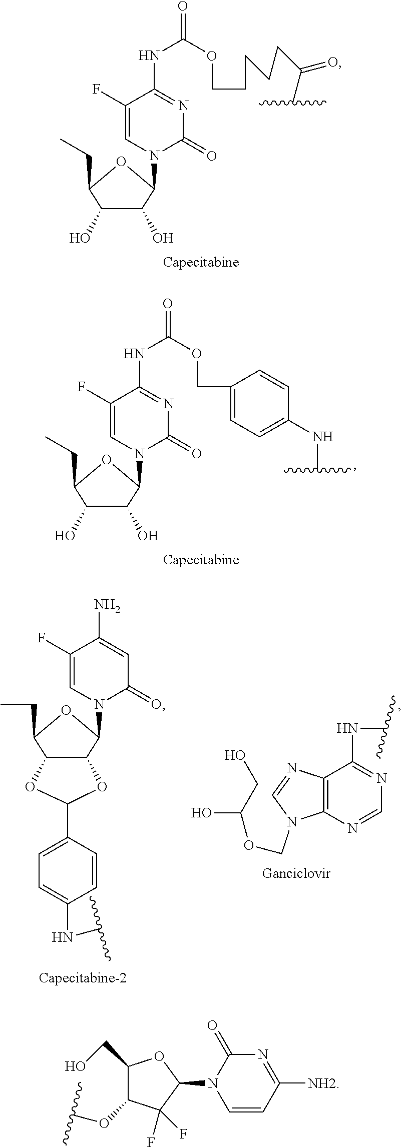

3. The antibody drug conjugate according to claim 1, wherein the cytotoxic drug, D₁ and D₂, are independently selected from:
(1), Chemotherapeutic agents:
a). an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, tricthylcnemelaminc, trictylenephosphoramide, tricthylencthiophosphoramidc and trimethylolomelamine];
b). A plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; erbulins, a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid}; and Inhibitors of nicotinamide phosphoribosyltransferase (NAMPT);
e). A hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (comprising CB 1093, EB 1089 KH 1060, cholecalciferol, ergocalciferol); Photodynamic therapies: (comprising verteporfin, phthalocyaninc, photosensitizer Pc4, demethoxyhypocrellin A); Cytokines: (comprising Interferon-alpha, Interferon-gamma, tumor necrosis factor (TNFs), human proteins containing a TNF domain)]};
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, neratinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group consisting of olaparib, niraparib, iniparib, talazoparib, veliparib, CEP 9722 (Cephalon's), E7016 (Eisai's), BGB-290 (BeiGene's), or 3-aminobenzamide;
h). An antibiotic, selected from the group consisting of an enediyne antibiotic (selected from the group consisting of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins andl-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca²+ATPase inhibitors or thapsigargin, Histone deacetylase inhibitors ((comprising Vorinostat, Romidepsin, Panobinostat, Valproic acid, Mocetinostat (MGCDO103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; razoxane; rhizoxin; sizofiran; spirogermanium; tenuazonic acid; triaziquone; 2, 2′,2″-trichlorotriethylamine; trichothecenes (including the group consisting of T-2 toxin, verrucarin A, roridin A and anguidine); urethane, siRNA, antisense drugs;
(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.
(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem/cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefinenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). Glycopeptides: bleomycin, vancomycin (oritavancin, telavancin), teicoplanin (dalbavancin), ramoplanin;
g). Glycylcyclines: tigecycline;
h). β-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
1). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). Other antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropencm, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddl), elvucitabine, emtricitabine (FTC), entecavir, famciclovir, fluorouracil (5-FU), 3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.
(5). A radioisotope that can be selected from the group consisting of (radionuclides)³H, ¹¹C, ¹⁴C, ¹⁸F, ³²p, ³⁵S, ⁴Cu, ⁶⁸Ga, ⁸⁶Y, ⁹⁰Y ⁹⁹Tc, ¹¹¹In, ¹²³I ¹²⁴I, ¹²⁵I, ¹³¹I, ¹³³Xe, ¹⁷⁷Lu, ²⁰³Pb, ²¹²Pb, ²¹¹At, ²¹³Bi, ²²⁴Ra and ²²⁵Ac. And the metal radionuclides, ⁶⁴Cu, ⁶⁸Ga, ⁸⁶Y, ⁹⁰Y ⁹⁹Tc, ¹¹¹In, ¹³³Xe ¹⁷⁷Lu, ²⁰³Pb, ²¹²Pb, ²¹¹At, ²¹³Bi, ²²⁴Ra and ²²⁵Ac are linked through a chelator and a linker L₁ of this patent application. The chelator is selected from: 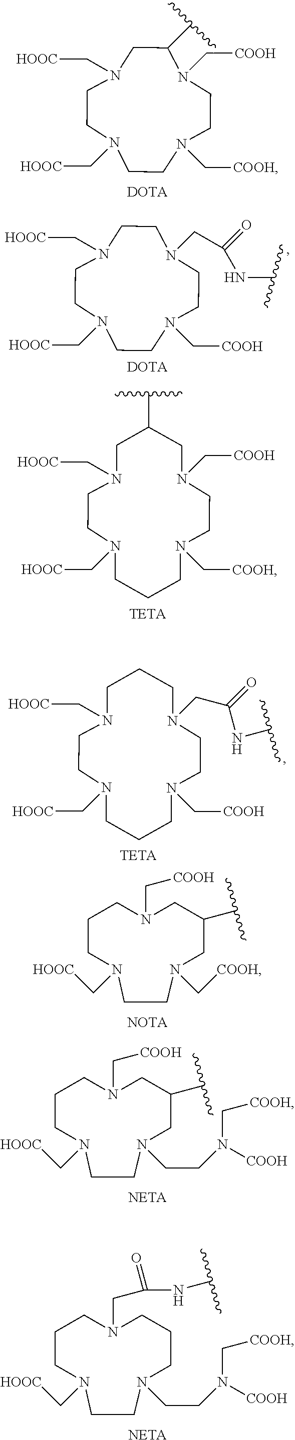 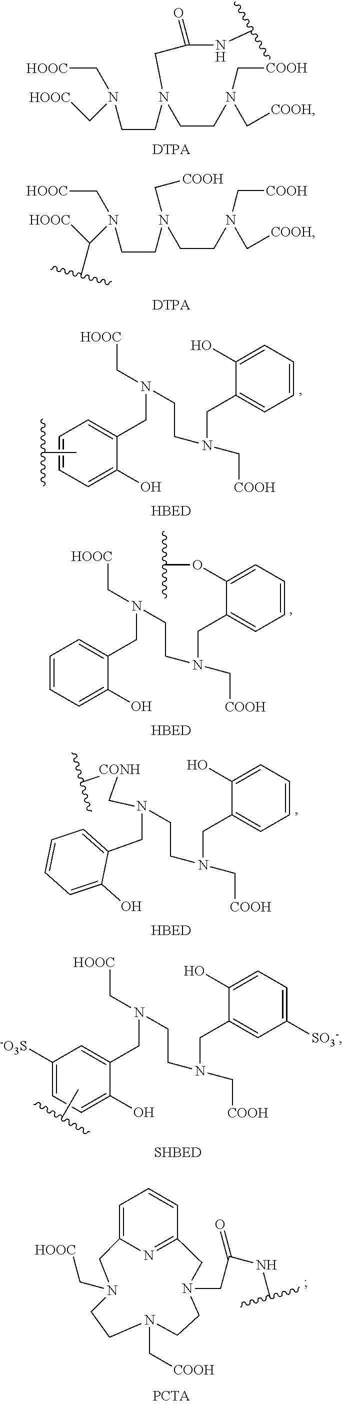 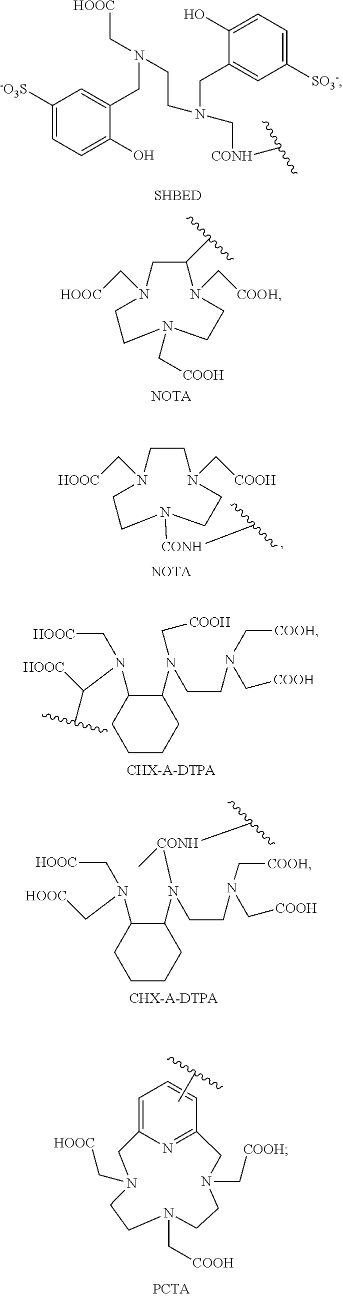
(6). A chromophore molecule, which is capable of absorbing UV light, florescent light, IR light, near IR light, visual light; A class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; Non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170). Acridine derivatives (comprising proflavin, acridine orange, acridine yellow). Arylmethine derivatives (comprising auramine, crystal violet, malachite green). Tetrapyrrole derivatives (comprising porphin, phthalocyanine, bilirubin); Any analogs and derivatives of the following fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin(PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; A fluorophore compound: comprising DCFH (2′7′Dichorodihydro-fluorescein, oxidized form), DHR (Dihydrorhodamine 123, oxidized form, light catalyzes oxidation), Fluo-3 (AM ester. pH >6), Fluo-4 (AM ester. pH 7.2), Indo-1 (AM ester, low/high calcium (Ca2+)), SNARF(pH 6/9), Allophycocyanin(APC), AmCyanl (tetramer, Clontech), AsRed2 (tetramer, Clontech), Azami Green (monomer), Azurite, B-phycoerythrin (BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRedl, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspbcrry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreenl (tetramer), ZsYellowl (tetramer) and their derivatives;
(7). The cell-binding ligands or receptor agonists, which can be selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (a-MSH); Cholecystokinins (CCK)/gastrin receptor agonists; Bombesins (selected from the group consisting ofPyr-Gln-Arg-Leu-Gly-Asn-Gln-Trp-Ala-Val-Gly-His-Leu-Met-NH₂)/gastrin-releasing peptide (GRP); Neurotensin receptor ligands (NTR₁, NTR2, NTR3); Substance P (NKI receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRR₈), selected from the group consisting of Toll-like receptors' (TLR₈) ligands, C-type lectins and Nodlike Receptors' (NLR₈) ligands; Calcitonin receptor agonists; integrin receptors' and their receptor subtypes' (selected from the group consisting of αᵥβ₁, αᵥβ₃, αᵥβ₃s, αᵥβ₆, a604, a7 μl, α_{L}β₂, α_{IIb}β₃) agonists (selected from the group consisting of GRGDSPK, cyclo(RGDfV) (L1) and its derives[cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists; an immunotherapeutical short antibody-like protein, siRNA or DNA molecule;
(8). The pharmaceutically acceptable salts, acids, derivatives, hydrate or hydrated salt; or a crystalline structure; or an optical isomer, racemate, diastereomer or enantiomer of any of the above drugs.

4. The antibody drug conjugate according to claim 1, wherein the cytotoxic drug, D₁ and D₂, are independently selected from:
a tubulysin and its analogs, a maytansinoid and its analogs, a taxanoid (taxane) and its analogs, a CC-1065 and its analogs, a daunorubicin or doxorubicin and its analogs, an amatoxin and its analogs, a benzodiazepine dimer (including dimers of pyrrolobenzodiazepine (PBD), tomaymycin, anthramycin, indolinobenzodiazepines, imidazobenzothiadiazepines, or oxazolidinobenzo-diazepines) and their analogs, a calicheamicin or an enediyne antibiotic and its analogs, an actinomycin and its analogs, an azaserine and its analogs, a bleomycin and its analogs, an epirubicin and its analogs, a tamoxifen and its analogs, an idarubicin and its analogs, a dolastatin and its analogs, an auristatin (including monomethyl auristatin E (MMAE), MMAF, auristatin PYE, auristatin TP, Auristatins 2-AQ, 6-AQ, EB (AEB), and EFP (AEFP)) and its analogs, a combretastatin, a duocarmycin and its analogs, a camptothecin and its analogs, a geldanamycin and its analogs, a methotrexate and its analogs, a thiotepa and its analogs, a vindesine and its analogs, a vincristine and its analogs, a hemiasterlin and its analogs, a nazumamide and its analogs, a spliceostatin, a pladienolide, a microginin and its analogs, a radiosumin and its analogs, an alterobactin and its analogs, a microsclerodermin and its analogs, a theonellamide and its analogs, an esperamicin and its analogs, PNU-159682 and its analogs, a protein kinase inhibitor, a MEK inhibitor, a KSP inhibitor, a nicotinamide phosphoribosyltransferase (NAMPT) inhibitor, an immunotoxin, a cell receptor agonist, a cell stimulating molecule or intracellular signaling molecule, one, two or more DNA, RNA, mRNA, small interfering RNA (siRNA), microRNA (miRNA), and PIWI interacting RNAs (piRNA), and stereoisomers, isosteres, analogs, or derivatives thereof;
wherein:
(a) Tubulysin analog having the following formula (IV): 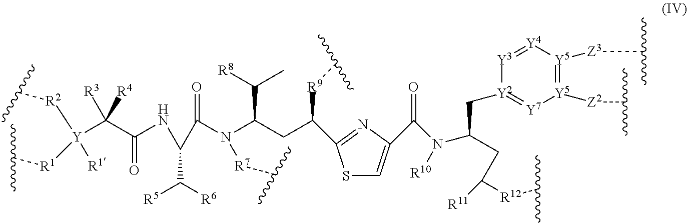
or a pharmaceutically acceptable salt, hydrates, or hydrated salt; or a polymorphic crystalline structure; or an optical isomer, racemate, diastereomer or enantiomer thereof,
wherein [symbol] is a linkage site that either one or two of them can link to L₁ and/or L₂ independently;
when two of “ [symbol] ”link to both L₁ and L₂, R¹ and R², or Z² and Z³ are preferably the dual linkage sites;
wherein R¹, R^{1′}, R², R³, and R⁴ are independently H, C₁-C₈alkyl; C₂~C₈ heteroalkyl, or heterocyclic;
C₃-C₈ aryl, Ar-alkyl, cycloalkyl, alkylcycloalkyl, heterocycloalkyl, heteroalkylcycloalkyl, carbocyclic, or alkylcarbonyl; or R¹R₂, R¹R₃, R²R₃, R³R4, or RSR⁶ form a 3~7 membered carbocyclic, cycloalkyl, heterocyclic, heterocycloalkyl, aromatic or heteroaromatic ring system; R₁ and R² can be independently absent when they link to L₁ or L₂ independently or simultaneously, Y¹ is N or CH;
wherein R⁵, R⁶, R⁸, R¹⁰ and R¹¹ are independently H, or C₁-C₄ alkyl or heteroalkyl;
wherein R⁷ is independently H, R¹⁴, —R¹⁴C(═O)X¹R¹⁵; or —R¹⁴X¹R¹⁵; X¹ is O, S, S—S, NH, CH₂ or NR¹⁴;
wherein R⁹ is selected from H, OH, ═O, —OR¹⁴, —OC(═O)R¹⁴, —OC(═O)NHR¹⁴, —OC(═O) NR¹⁴R¹⁵, OP(═O)(OR¹⁴)₂, —OC(═O)NR¹⁴R¹⁵, or OR¹⁴OP(═O)(OR¹⁵)₂; when R⁹ links L1 or L₂, R⁹ is —O—, —OC(═O)NH—or —OC(═O)N(R¹⁴)—;
wherein R¹ is independently H, R¹⁴, —R¹⁴C(═O)R¹⁵, —R¹⁴C(═O)X²R¹⁵, wherein X² is —O—, —S—, —NH—, or —N(R¹⁴)—.
wherein R¹² is —COOH, —COSH, —CONH₂, CONHNH₂, CONHNHR″, —CONH(R′⁵), —COOR¹⁵, —R¹⁵COR¹⁶, —R¹⁵COOR¹⁶, —R¹⁵C(O)NH₂, —R¹⁵C(O)NHR¹⁶, —COSR¹⁵, R¹⁵S(═O)₂R¹⁶, —R′⁵P(═O)(OR¹⁷)₂, —R¹⁵OP(═O)(OR¹⁷)₂, —COOCH₂OP(═O)(OR¹⁷)₂, —COX²SO₂R¹⁷, —COOR¹⁵X²R¹⁶, tetrazole, imidazole, or triazole, where X² is —O—, —S—, —NH—, —N(R¹⁵)—, —O—R¹⁵—, —S—R¹⁵—, CH₂ or —NHR¹⁵—; when R¹² links L₁ or L₂, R¹² is —C(O)O—, —C(O)NH—, —C(═O)NHS(O)₂R¹⁵— or —C(═O)N(R⁵)—;
R¹³ and R¹⁴ are independently C₁~C₈ alkyl, heteroalkyl; C₂-C₈ of alkenyl, alkynyl, heteroalkyl, heterocycloalkyl; C₃-C₈ of aryl, Ar-alkyl;
Z² and Z³ are independently H, O, S, NH, N(R¹⁵), NHNH, —OH, —SH,-NH₂, NH, NHNH₂,-NH(R¹⁵), —OR′⁵, CO, —COX², —COX²R¹⁶, R¹⁷, F, Cl, Br, I, SR¹⁶, NR¹⁶R¹⁷, N═NR¹⁶, N═R¹⁶, NO₂, SOR¹⁶R¹⁷, SO₂R¹⁶, S O₃R¹⁶, OS O₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷; —O—(C₄-C₁₂ glycoside), —N-(C₄-C₁₂ glycoside); C₁~C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)₁₋₈, or CO(Aa)_{1~8} (which are respectively N-terminal or C-terminal 1-8 the same or different amino acids)), or polyethyleneoxy unit of formula (OCH₂CH₂)ₚ or (OCH₂CH(CH₃))ₚ, wherein p is an integer from 0 to about 1000, or combination of above groups thereof; X² is O, S, S—S, NH, CH₂, OH, SH, NH₂, CHR¹⁵ or NR″;
R¹⁵, R¹⁶ and R¹⁷ are independently H, C₁-C₈ alkyl, heteroalkyl; C₂-C₈ of alkenyl, alkynyl, heteroalkyl, heterocycloalkyl; C₃-C₈ of aryl, Ar-alkyl, cycloalkyl, heteroalkylcycloalkyl, alkylcarbonyl, heteroaryl, alkylcarbonyl, or Na⁺, K⁺, Cs⁺, L₁⁺, Ca²⁺, Mg⁺, Zn²⁺, N⁺(R¹)(R²)(R³) (R⁴), HN⁺(C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N (R₁), thus Y², Y⁴, Y⁵, Y⁶ and Y⁷form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y⁴, Y⁵, Y⁶ and Y⁷ form aromatic ring of benzene, pyridine, pyridazine, pyrimidine, pyrazine, triazine, tetrazine, pentazine;
the tubulysin analogs have the structures shown below: 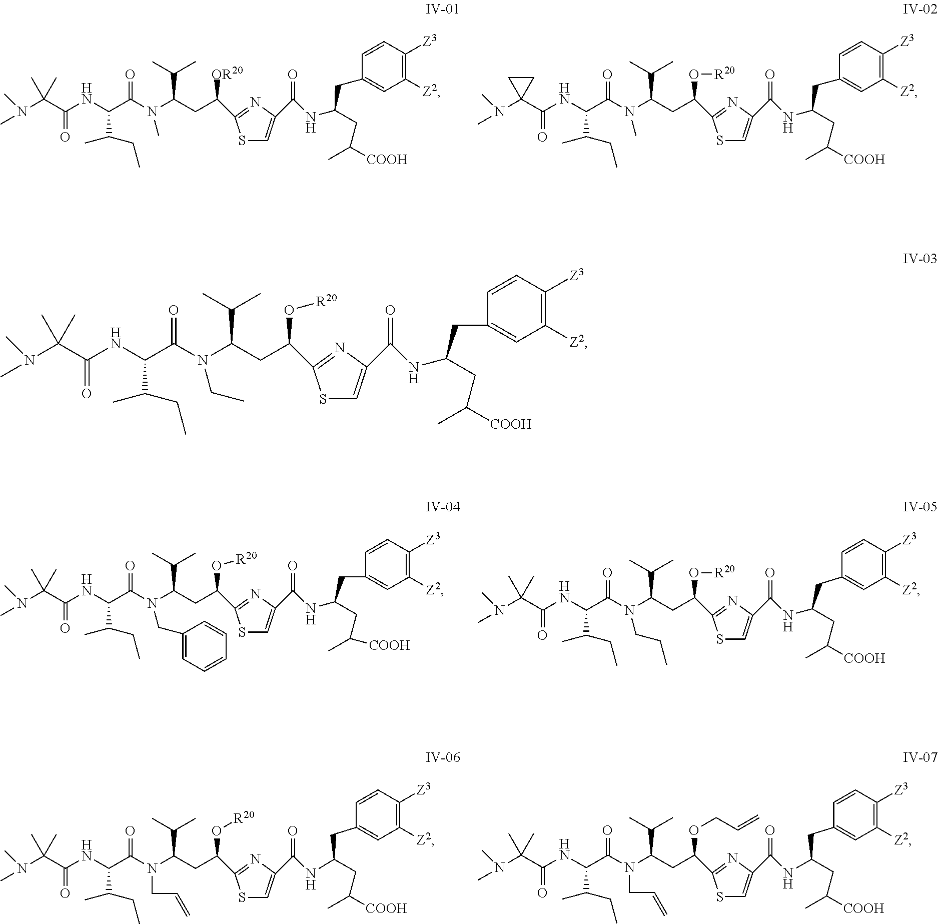 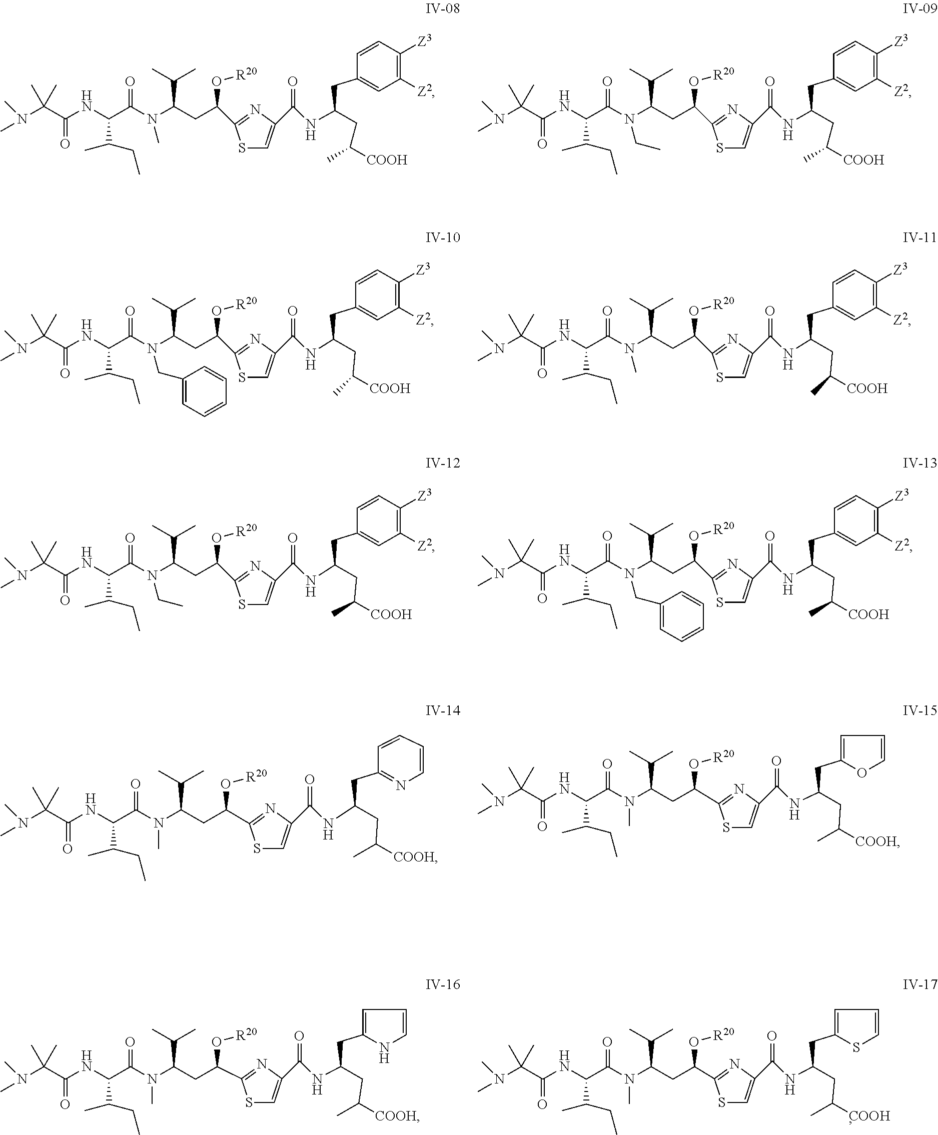 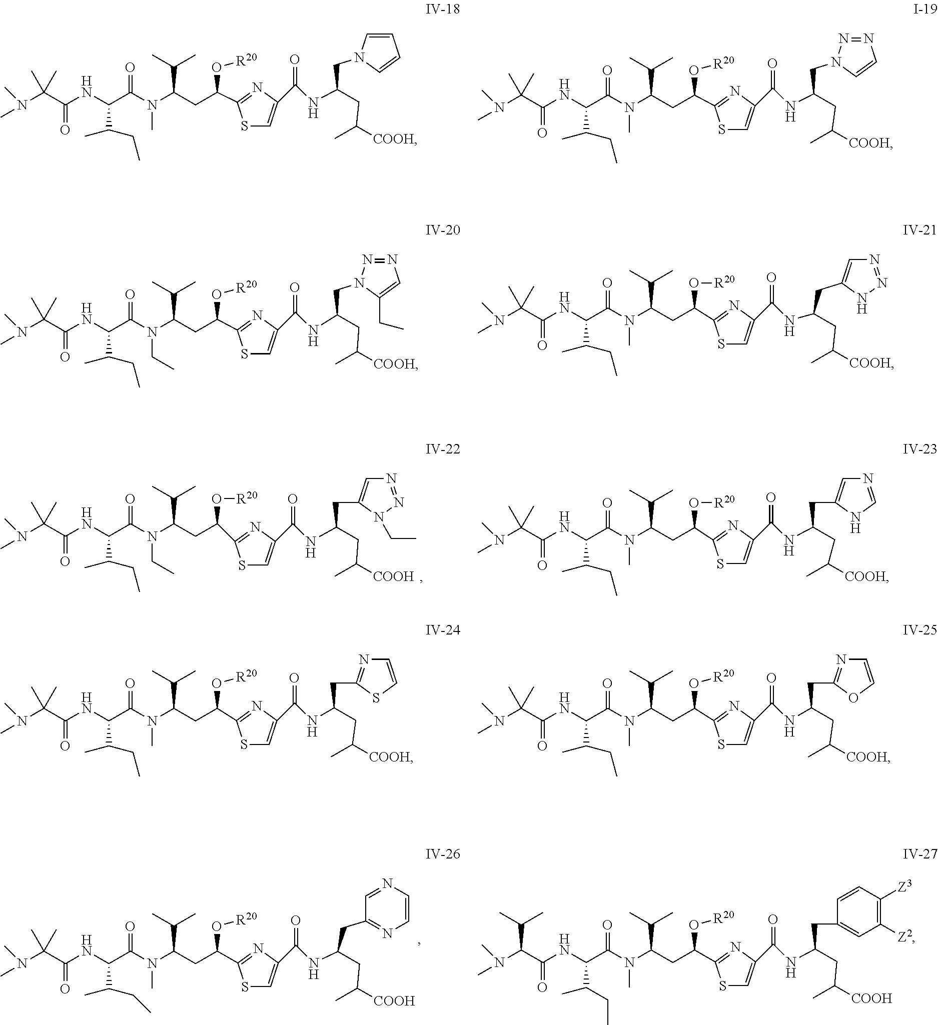 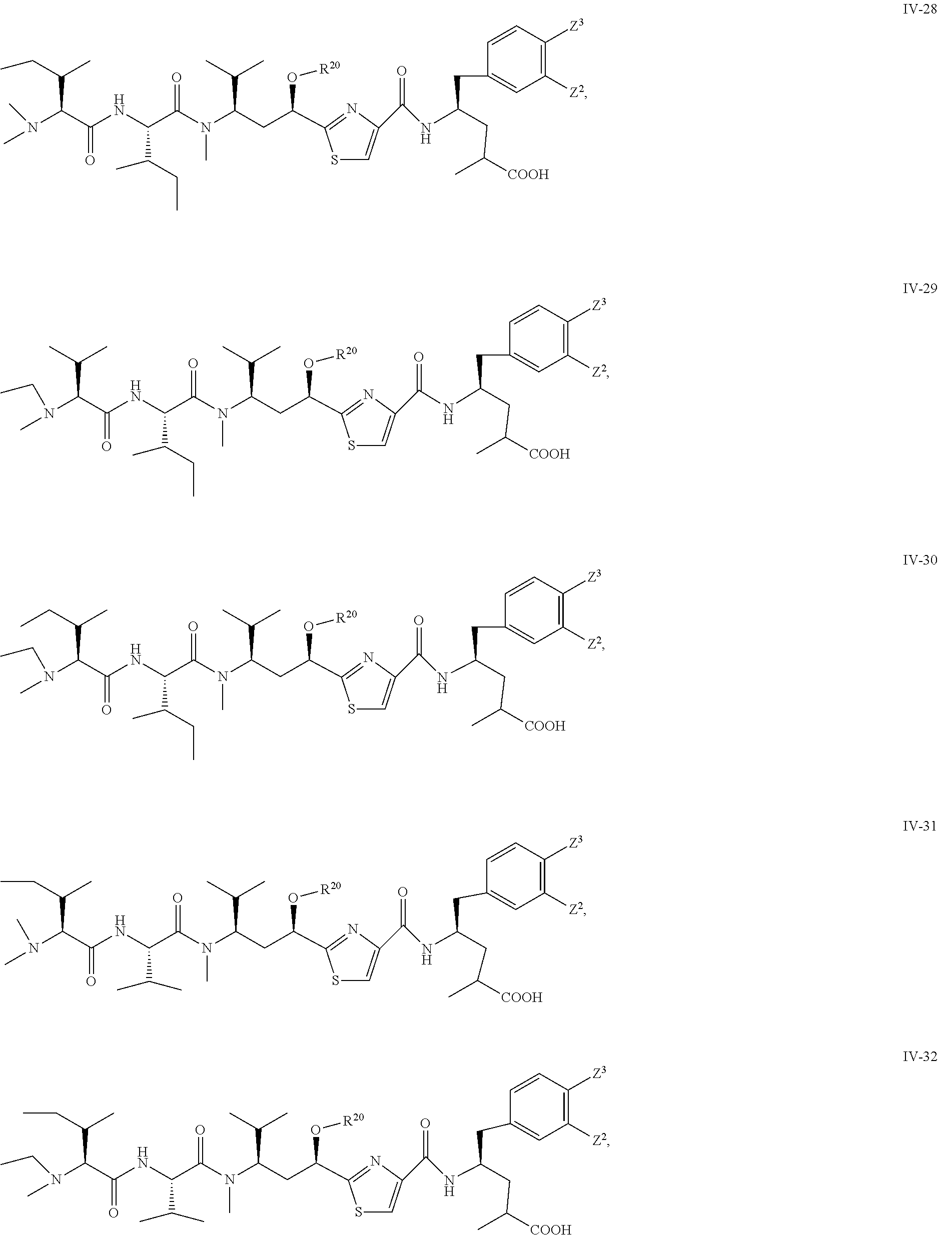 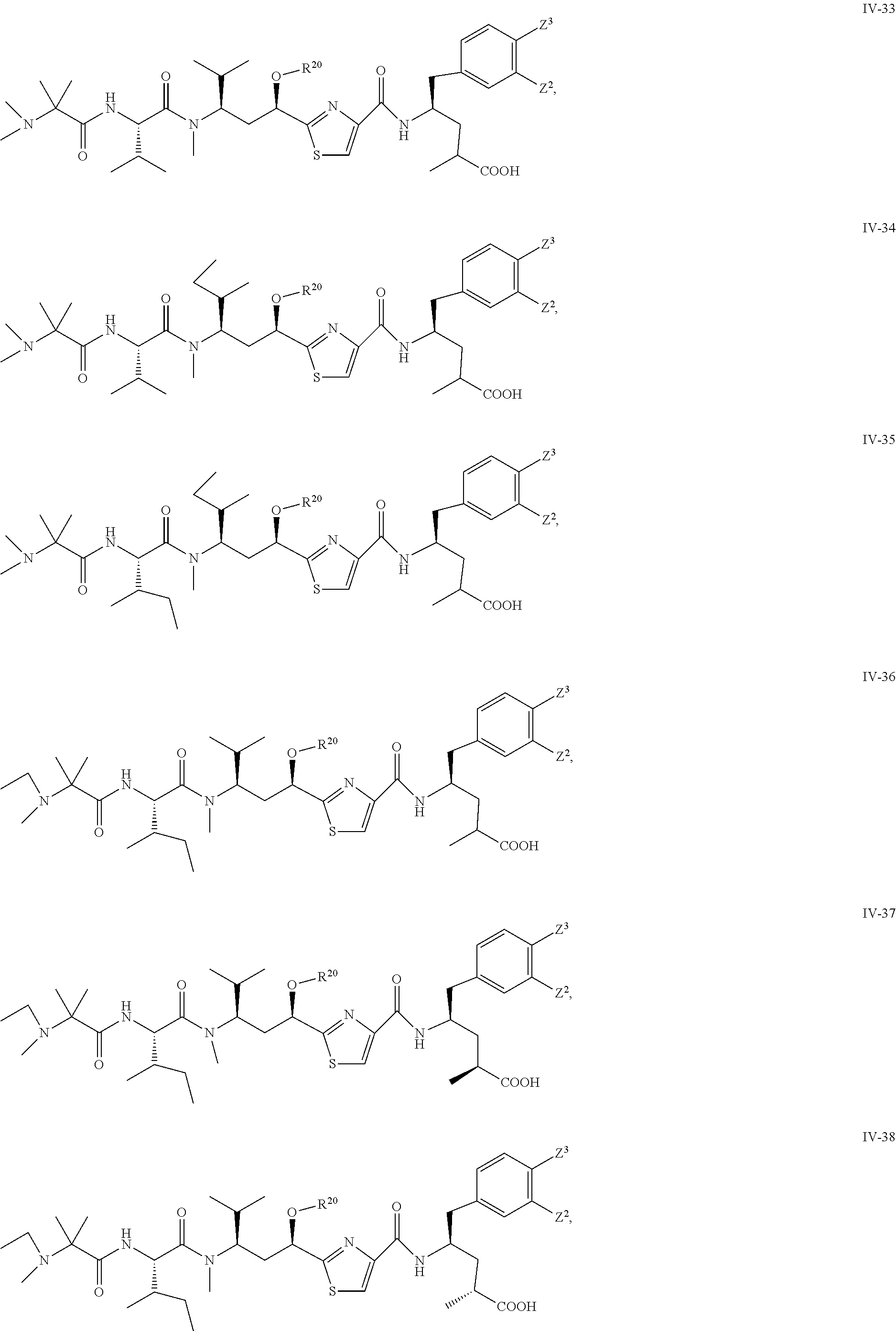 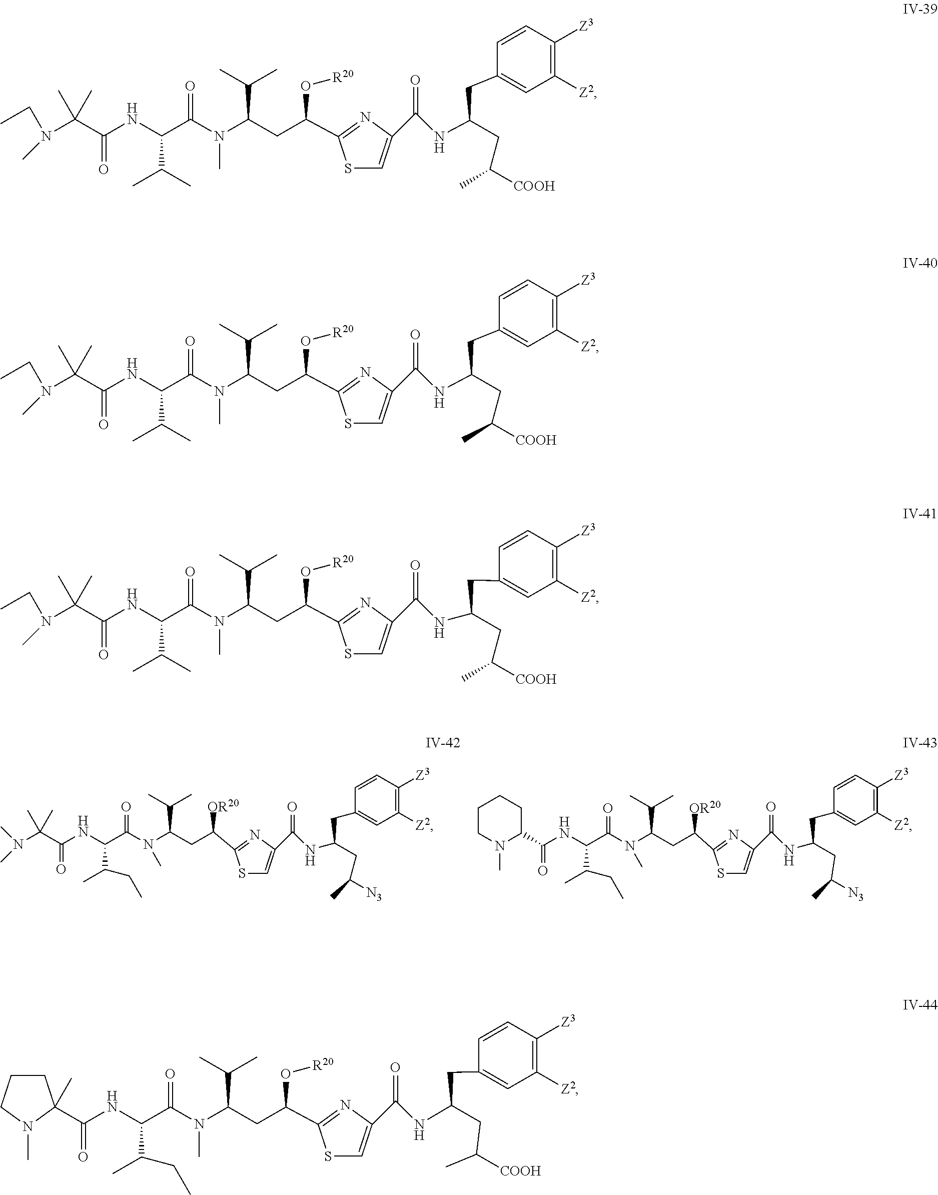 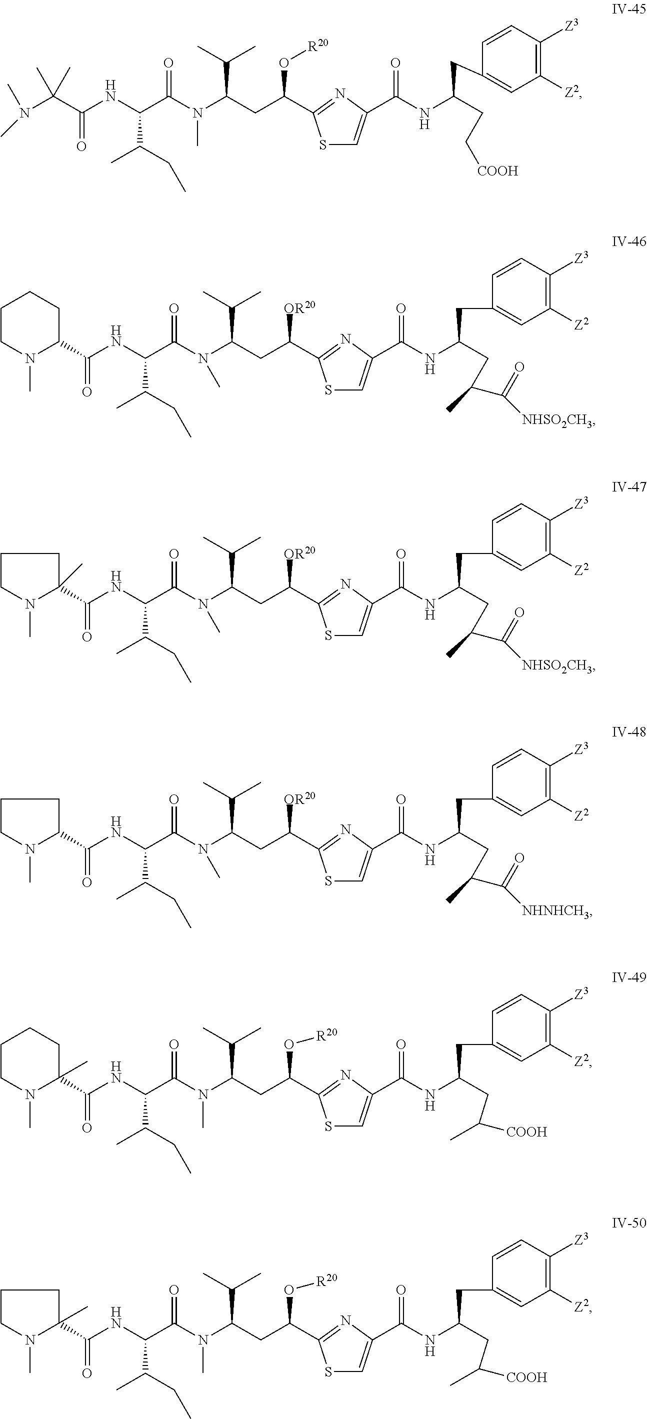 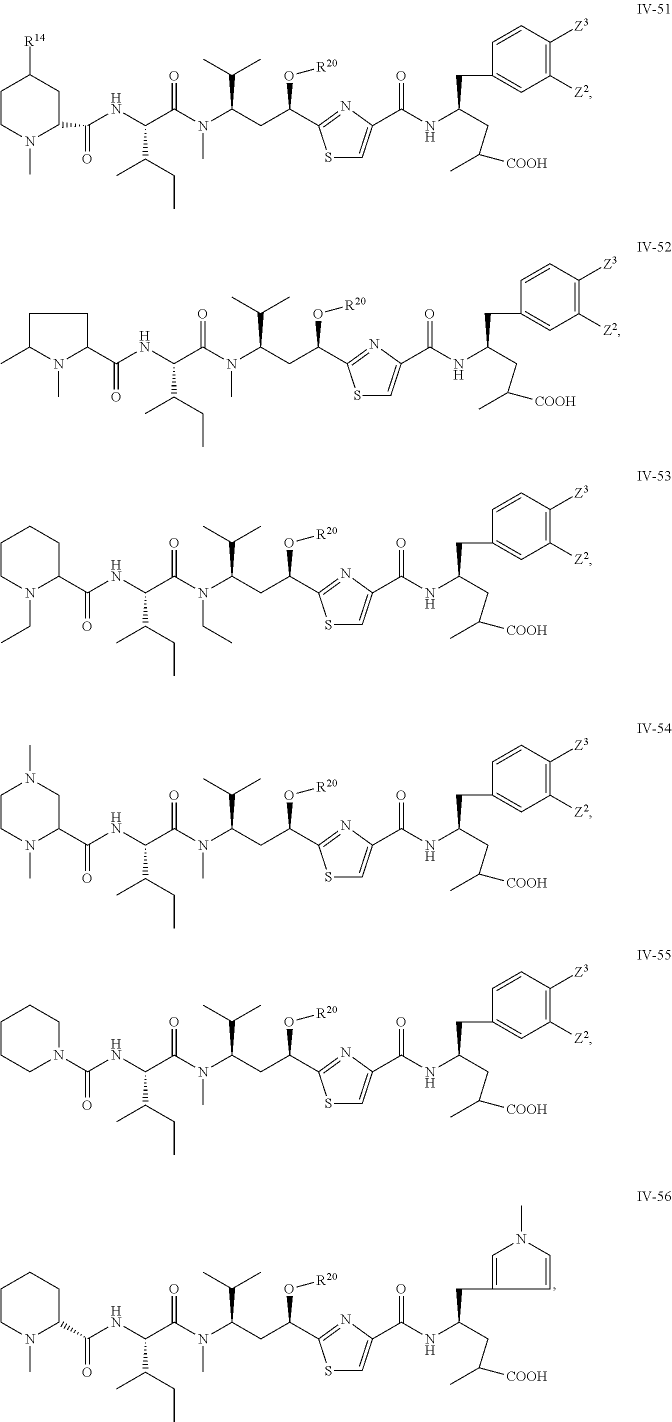 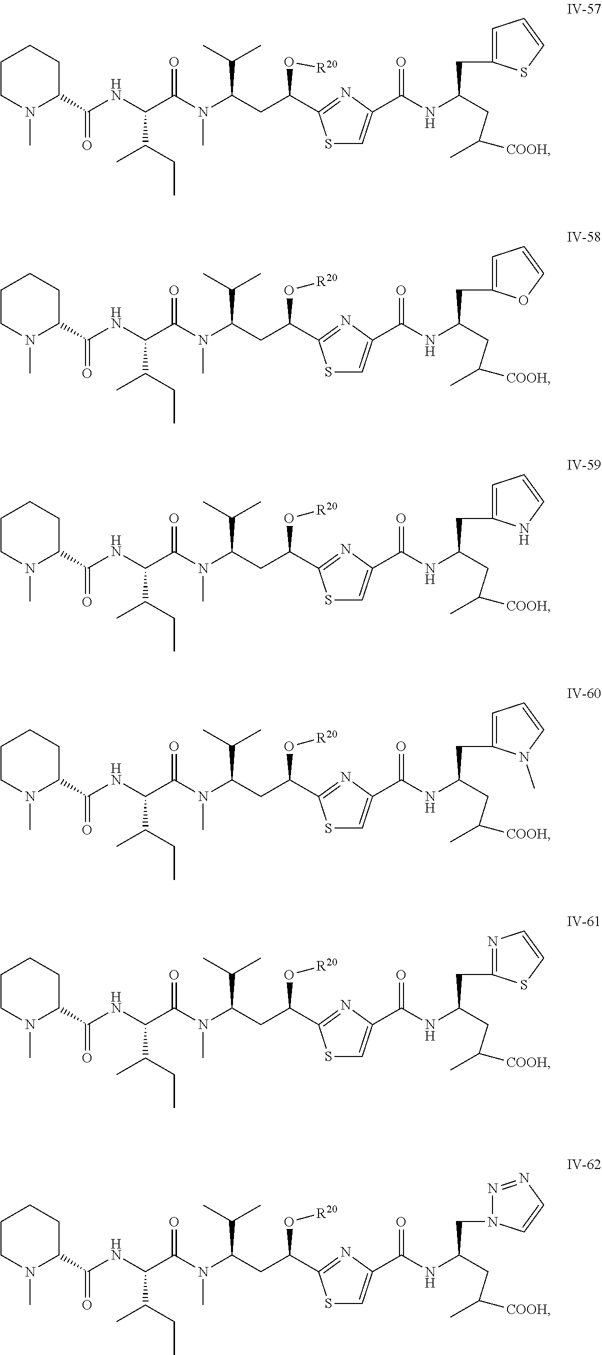 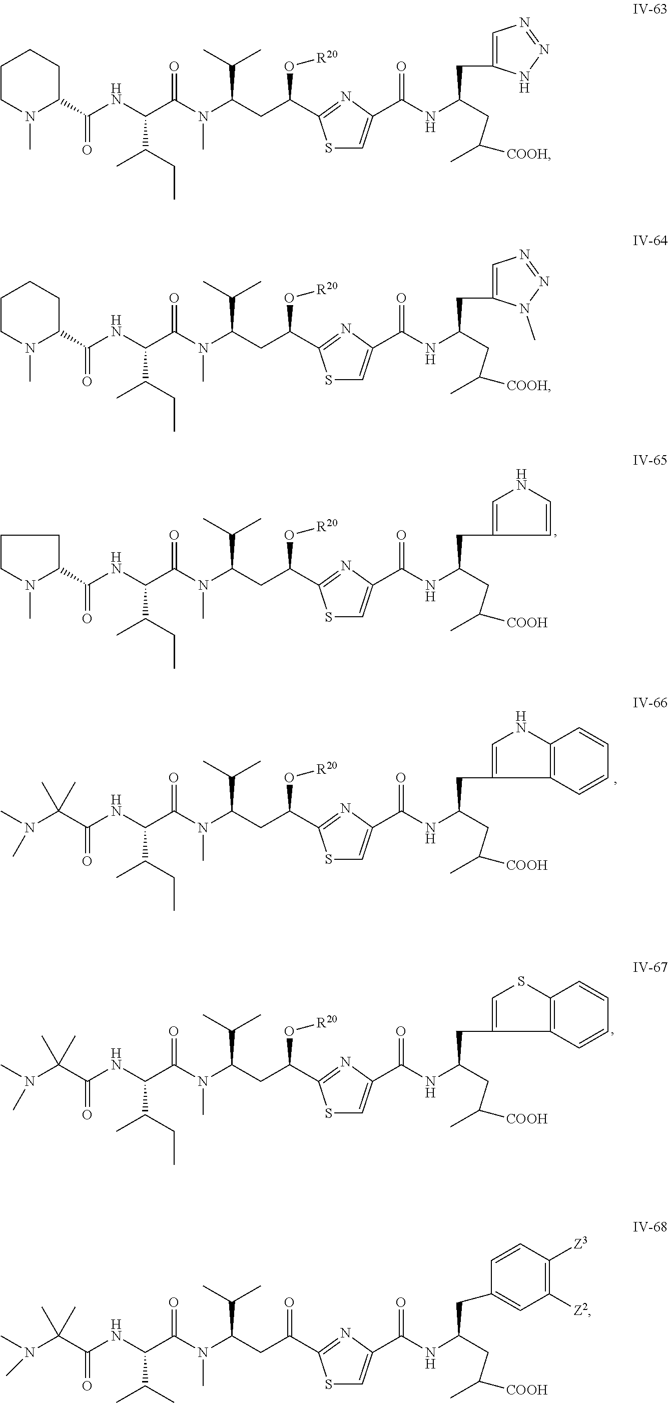 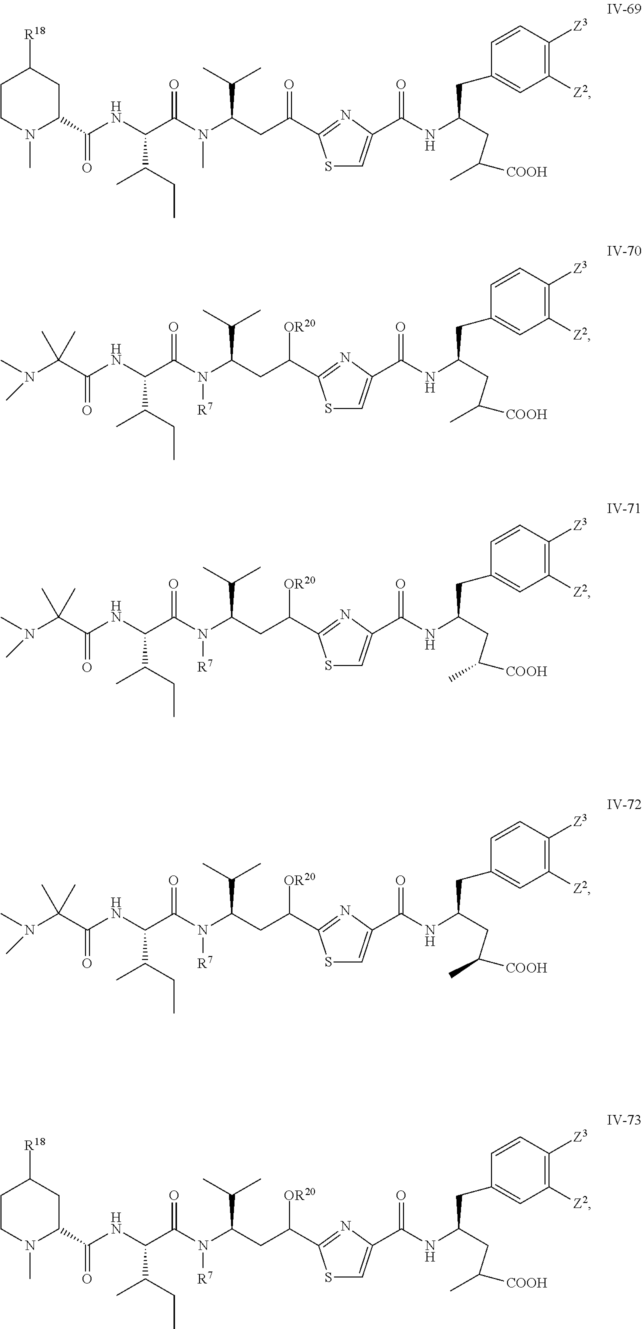 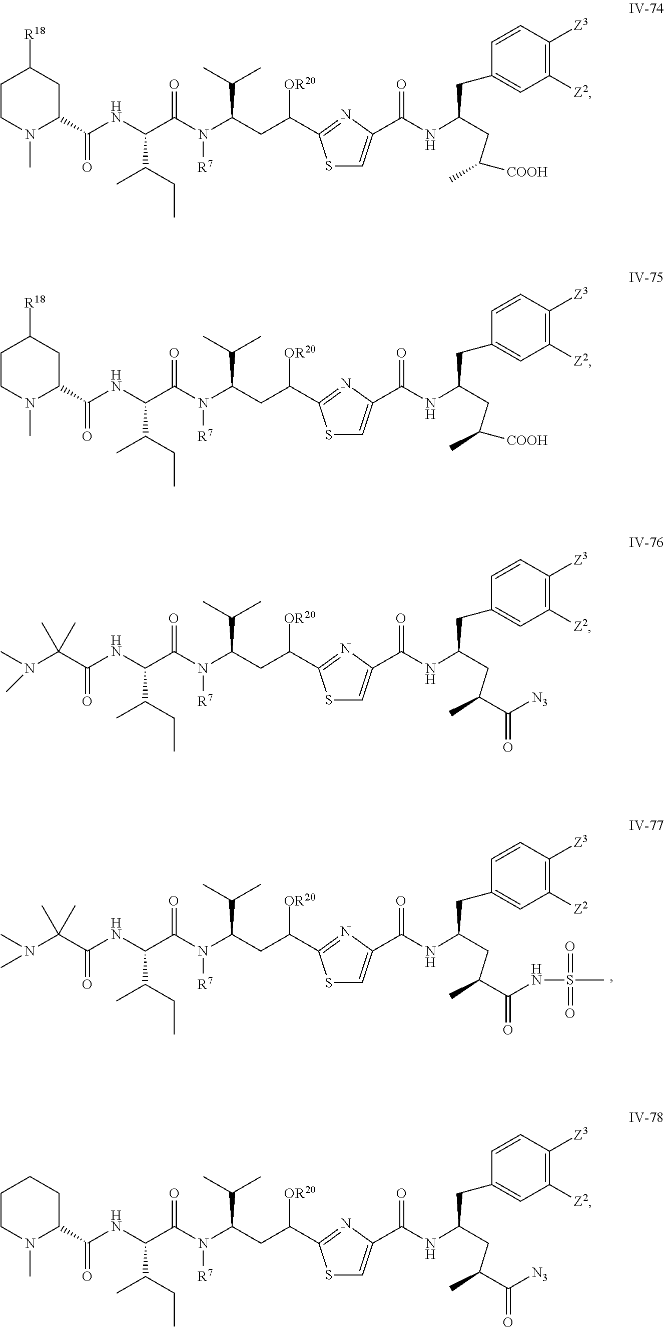 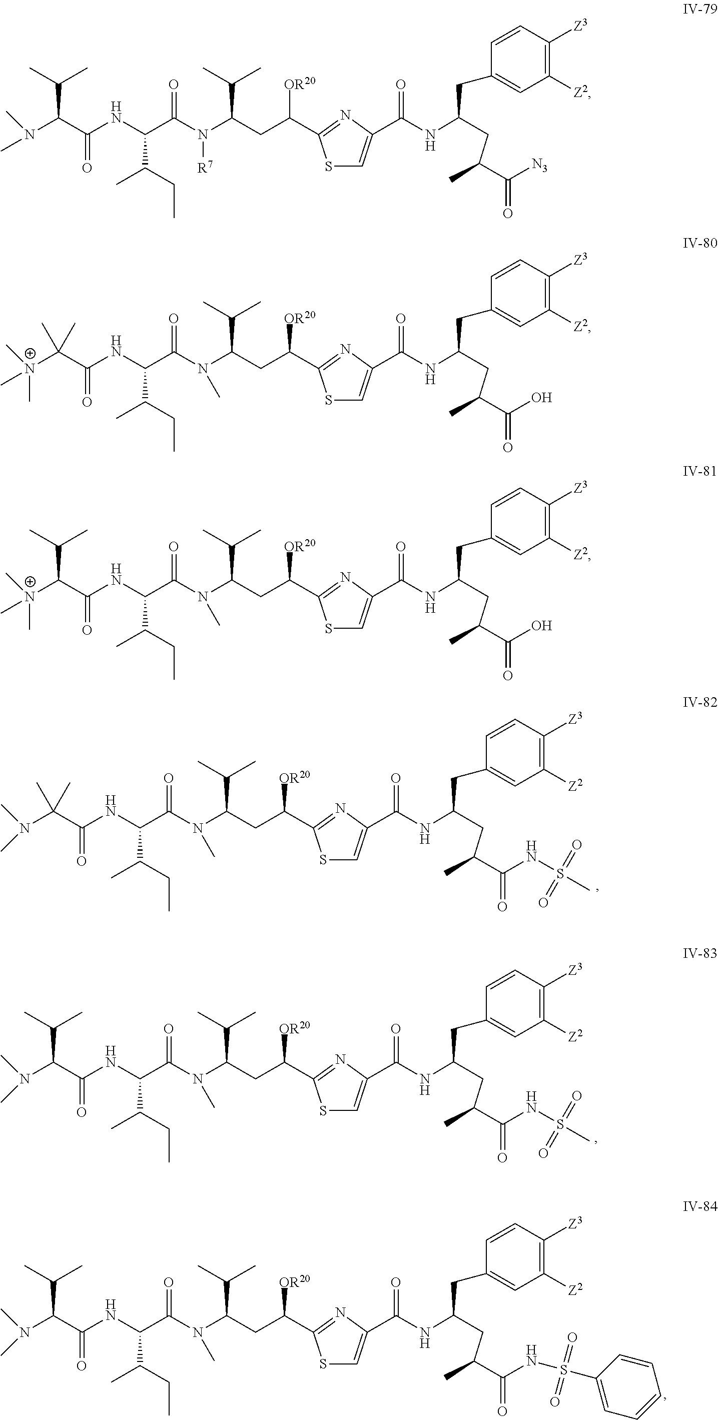 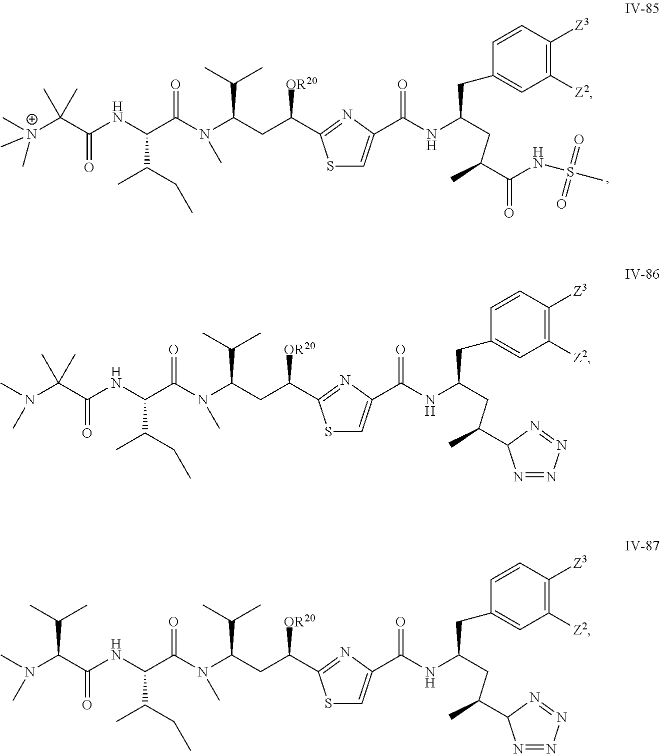 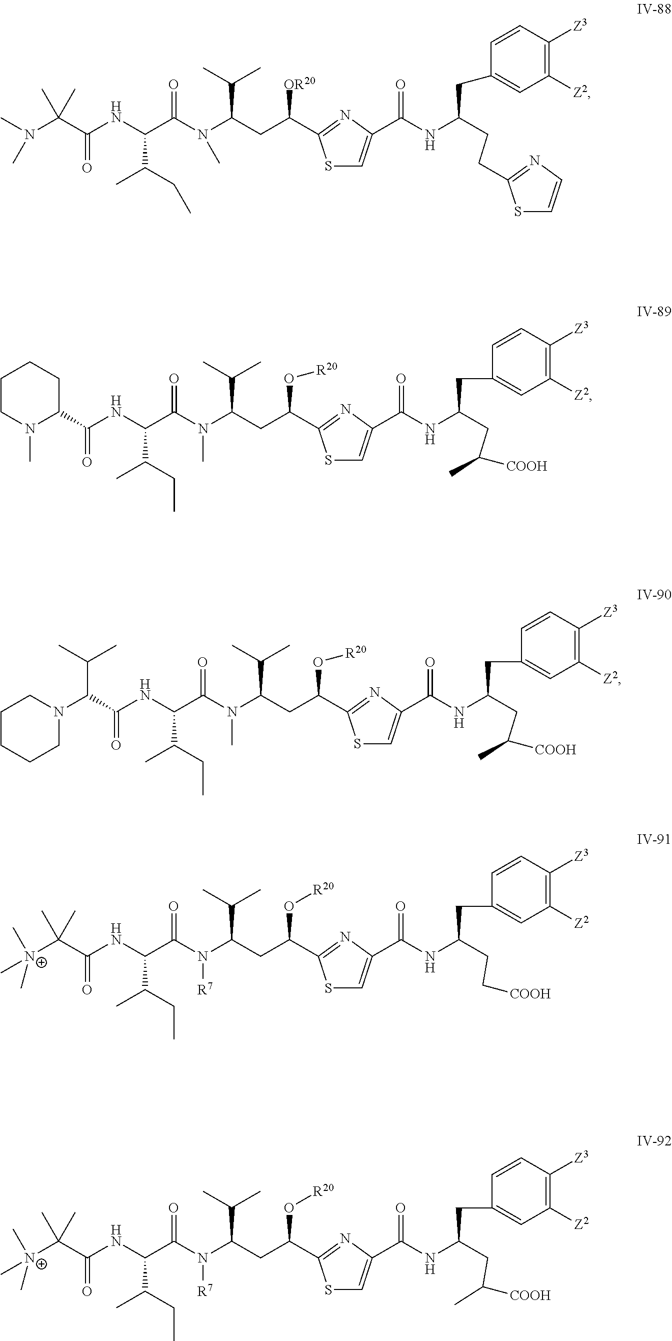 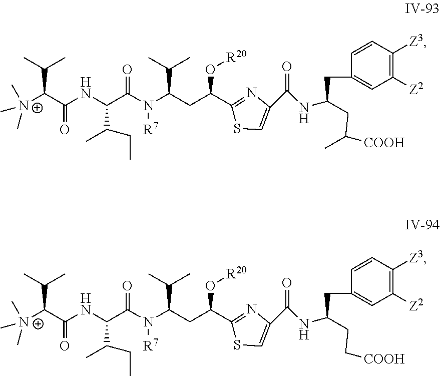
wherein R²⁰ is H; C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (-C(O)OR¹⁷), carbamate (—C(O)NR¹⁷R¹⁸); or 1-8 carbon atoms of carboxylate, esters, ether, or amide; or 1-8 amino acids; or polyethyleneoxy unit of formula (OCH₂CH₂)ₚ or (OCH₂CH(CH₃))ₚ, wherein p is an integer from 0 to about 1000; or R²⁰ is absent and the oxygen forms a ketone, or combination above groups;
Z³ and Z³ are independently H, OH, NH₂, O, NH, COOH, COO, C(O), C(O), C(O)NH, C(O)NH₂, R¹⁸, O CH₂OP(O)(OR¹⁸)₂, O C(O)OP(O)(OR¹⁸)₂, O PO(OR¹⁸)₂, N H PO(OR¹⁸)₂, O P(O)(OR¹⁸)OP(O)(OR¹⁸)₂, O C(O)R¹⁸, OC(O)NHR¹⁸, OSO₂(OR¹⁸), O—(C₄-C₁₂-glycoside), of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸);
R¹⁹ is H, OH, NH₂, OSO₂(OR¹⁸), XCH₂OP(O)(OR¹⁸)₂, XPO(OR¹S)₂, XC(O)OP(O)(OR¹⁸)₂, XC(O)R¹⁸, XC(O)NHR¹⁸, C₁~C₈ alkyl or carboxylate; C₂~C₈ alkenyl, alkynyl, alkylcycloalkyl, heterocycloalkyl; C₃-C₈ aryl or alkylcarbonyl; or pharmaceutical salts;
X is O, S, NH, NHNH, or CH₂;
R⁷ is defined the same above; wherein the linkage sites, “ [symbol] ” in formula IV-01-IV-79 are the same indication according to formula (IV);
(b) the calicheamicin and their related enediyne antibiotics have the following formula: 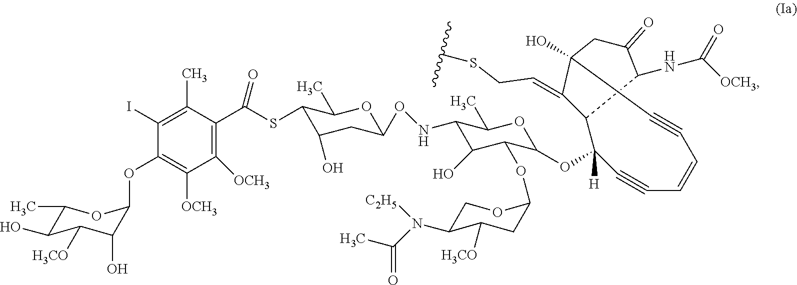 [SMILES: *SC/C=C1\C2[C@@H](OC3OC(C)C(NOC4CC(O)C(SC(=O)c5c(C)c(I)c(OC6OC(C)C(O)C(OC)C6O)c(OC)c5OC)C(C)O4)C(O)C3OC3CC(OC)C(N(CC)C(C)=O)CO3)C#C/C=C\C#C[C@]1(O)CC(=O)[C@@H]2NC(=O)OC]
or an isotope of a chemical element, or a pharmaceutically acceptable salt, hydrates, or hydrated salt; or a polymorphic crystalline structure; or an optical isomer, racemate, diastereomer or enantiomer thereof,
wherein [symbol] is the site linked to L₁ or L₂;
(c) Geldanamycins are benzoquinone ansamycin antibiotic which are 17-AAG (17-N-Allylamino-17-Demethoxygeldanamycin) and 17-DMAG (17-Dimethylaminoethylamino-17-demethoxygeldanamycin) having the following formula: 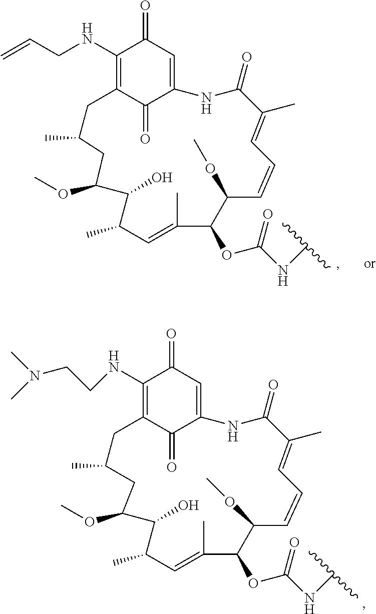
wherein [symbol] is the site linked to L₁ or L₂;
(d) the Maytansines or their derivatives maytansinoids have the following formula: 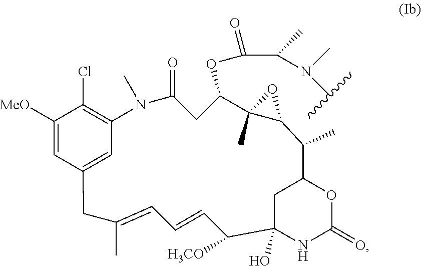
wherein [symbol] is the site linked to L₁ or L₂.
(e) the camptothecin (CPTs) and its derivatives have the following formula: 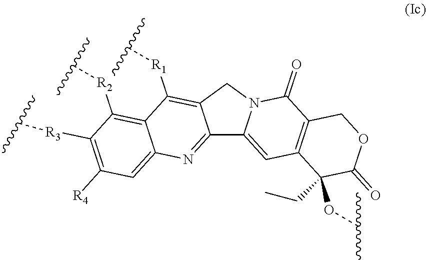
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein R₁, R₂ and R₄ are independently selected from H, F, Cl, Br, CN, NO₂, C₁~C₈ alkyl; O-C₁-C₈ alkyl; NH—C₁~C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, carbonate, urea, or carbamate;
R₃ is H, OH, NH₂, C₁-C₈alkyl; O—C₁~C₈ alkyl; NH—C₁~C₈ alkyl; C₂-C₈ of heteroalkyl, alkylcycloalkyl, heterocycloalkyl; or 2-8 carbon atoms of esters, ether, amide, carbonate, urea, or carbamate; or R₁R₂, R₂R₃ and R₃R₄ independently form a 5-7 membered carbocyclic, heterocyclic, heterocycloalkyl, aromatic or heteroaromatic ring system; [symbol] is the site in the molecule that can be linked to L₁ or L₂.
the camptothecin (CPTs) and its derivatives have the following formula: 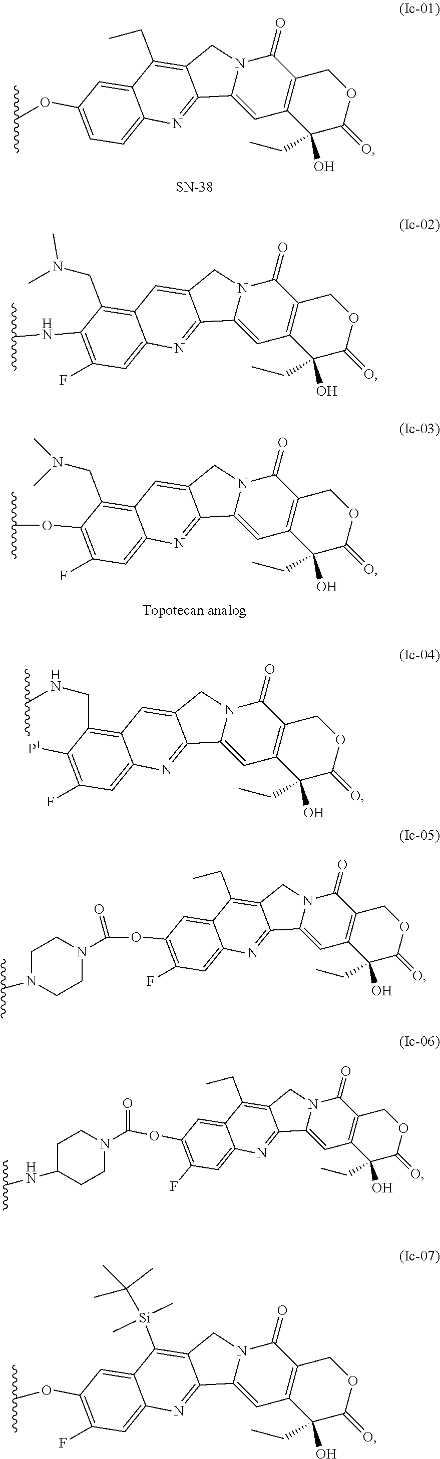 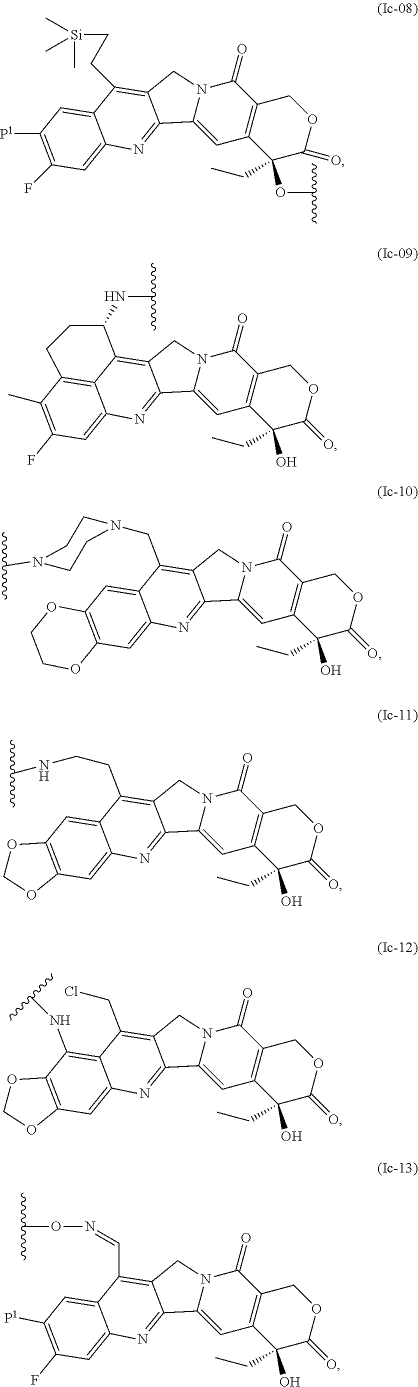 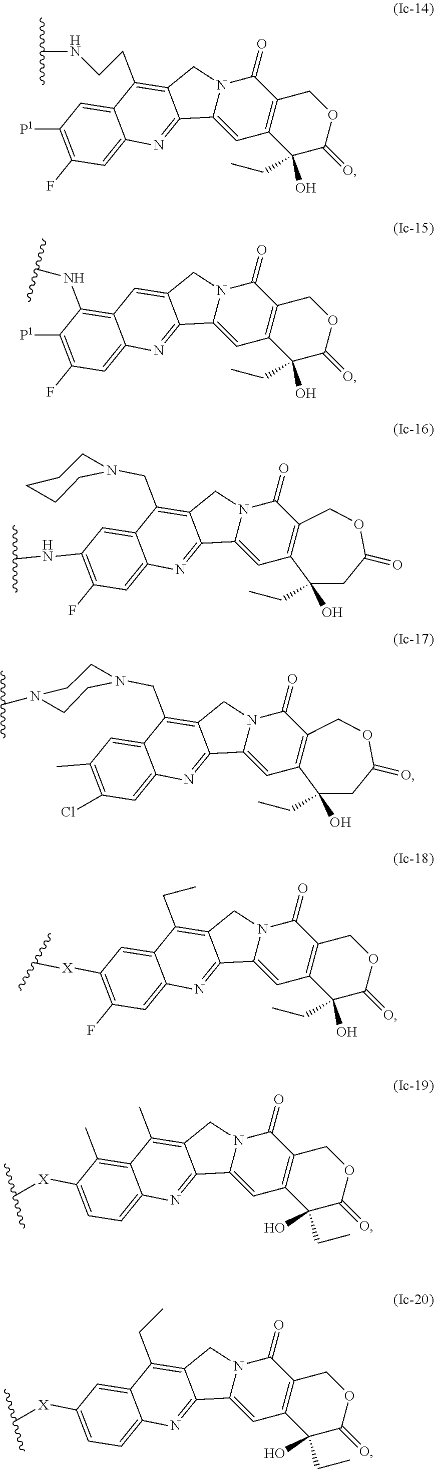 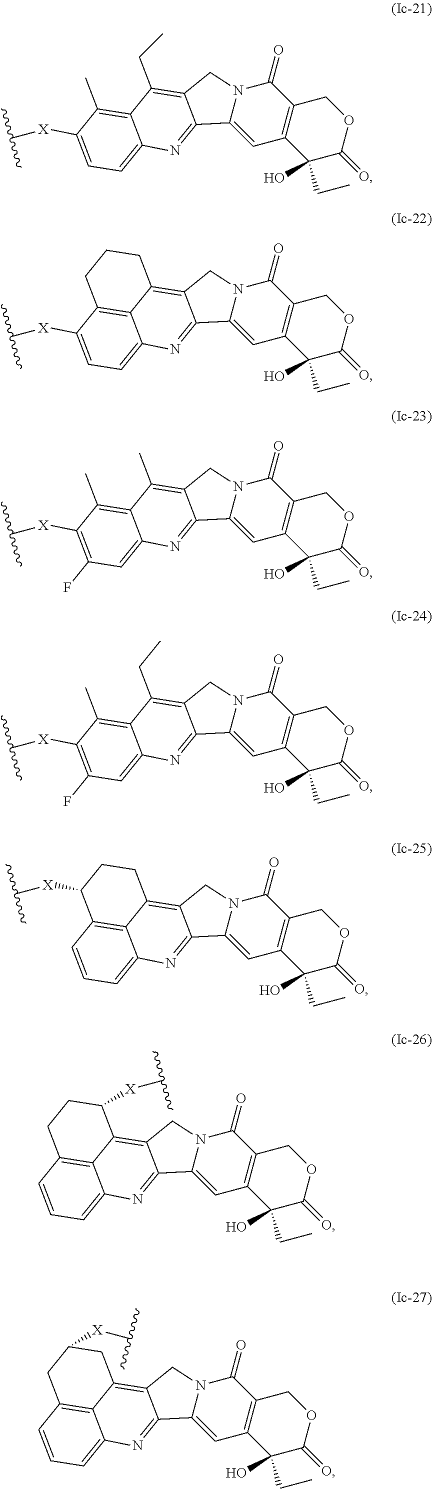 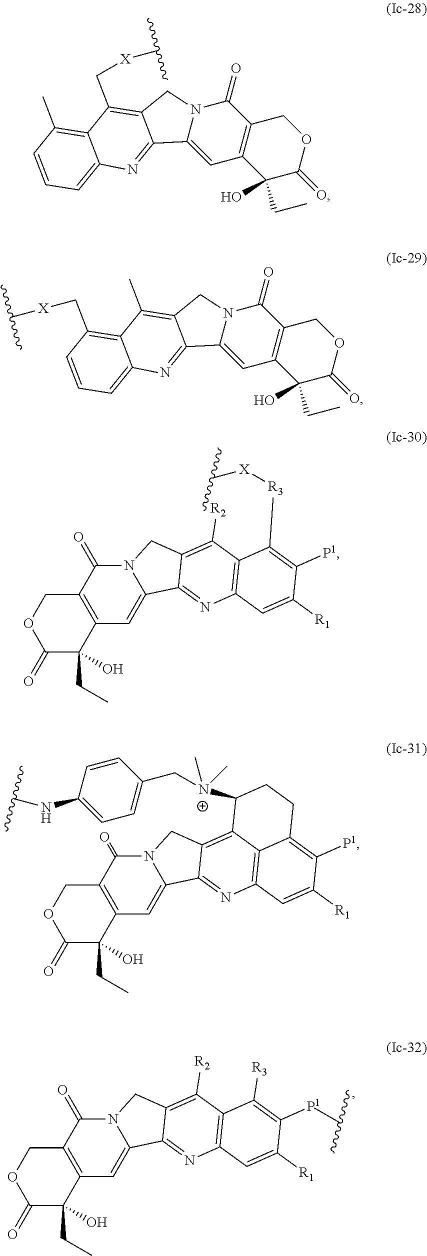 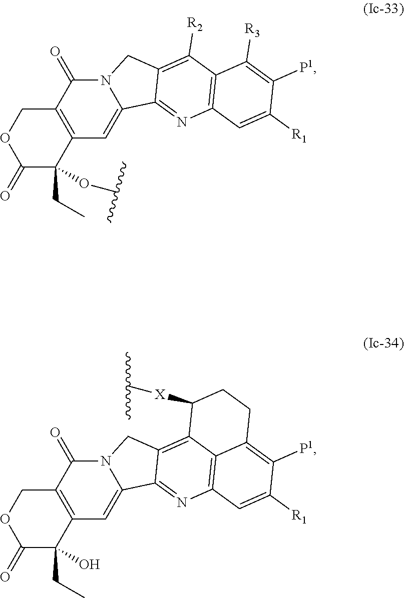
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein [symbol] is the site linked to L₁ or L₂;
P¹ and R₁ are independently H, F, Cl, Br, I, SO₂R₂, SO₃H, CN, OH, N H₂, COO H, C(O)N H₂, O CH₂OP(O)(OR¹⁸)₂, OC(O)OP(O)(OR¹⁸)₂, O PO(OR¹⁸)₂, N H PO(OR⁸)₂, O C(O)R¹⁸, OP(O)(OR′⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ of linear or branched alkyl), O—(C₁-C₈ of linear or branched alkyl)—OH, C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R₂ and R₃ are independently H, C₁-C₈ of linear or branched alkyl, or C₂-C₈ of linear or branched heteroalkyl, ether, amine, ester, or amide; in addition, R₂ and R₃ can be joint together to form five or six member ring or cycloalkyl, cycloalkylamine, or cycloalkylamide, cyclohexylalkylamide; R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); X is NH, O, S, S(O₂), NHS(O₂), NHS(O₂)NH, NHP(O)(OH), N+(R₂)(R₃), NHC(O)NH, NHC(O), NHC(O)O, N(CH₂CH₂)₂N, CON(CH₂CH₂)₂N, CON(CH₂CH₂)₂N+(R₂)(R₃), or CH₂;
(f) the Combretastatins have the following formula: 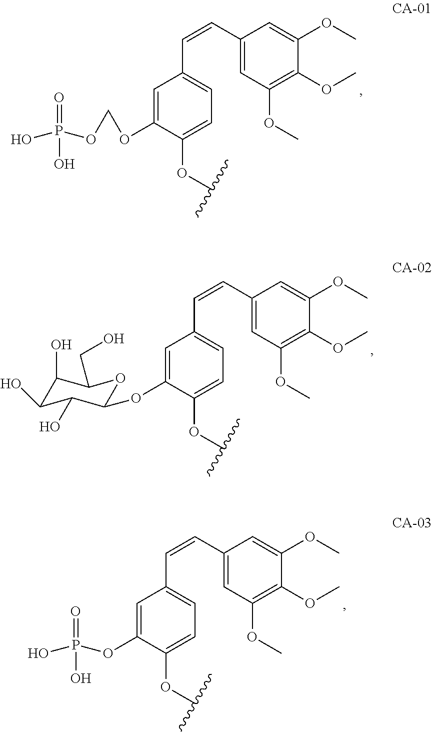 [SMILES: *Oc1ccc(/C=C\c2cc(OC)c(OC)c(OC)c2)cc1OCOP(=O)(O)O ; *Oc1ccc(/C=C\c2cc(OC)c(OC)c(OC)c2)cc1OC1OC(CO)C(O)C(O)C1O ; *Oc1ccc(/C=C\c2cc(OC)c(OC)c(OC)c2)cc1OP(=O)(O)O]
(g) the Taxanes have the following formula: 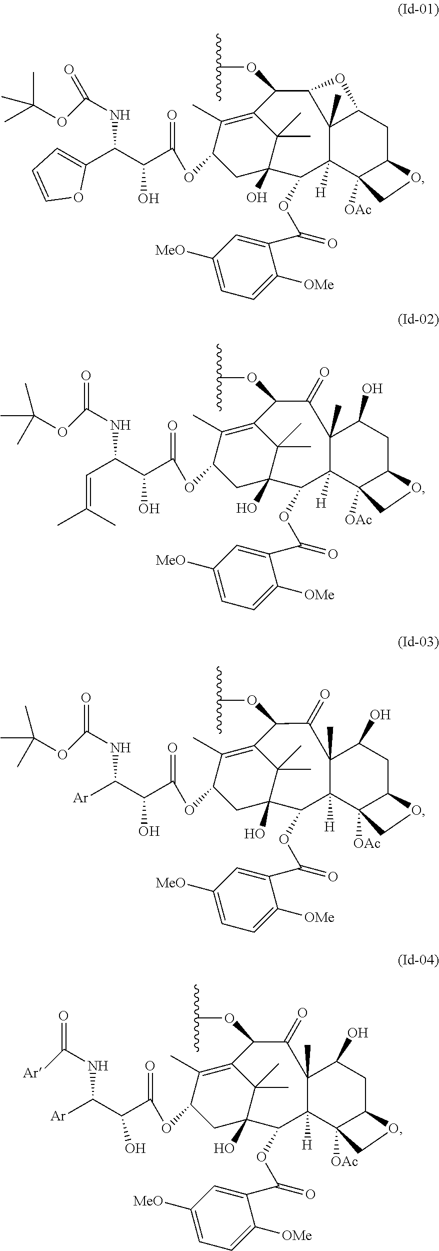
wherein [symbol] is the site linked to L₁ or L₂; Ar and Ar′ are independently aryl or heteroaryl;
(h) the anthracyclines have the following formula: 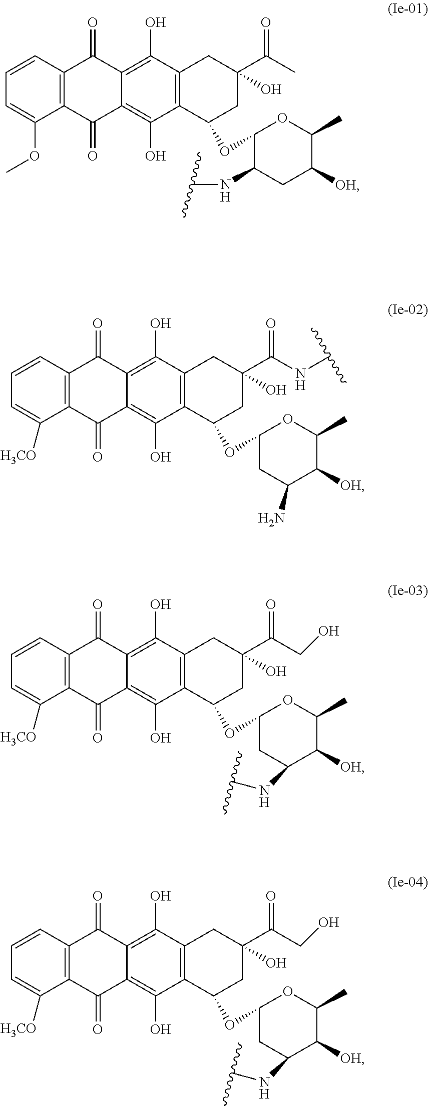 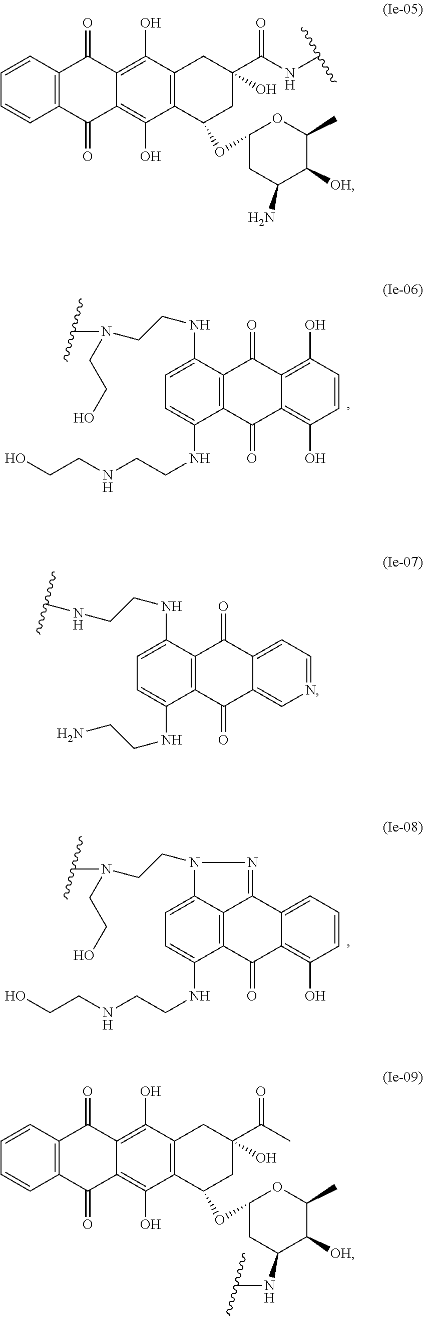 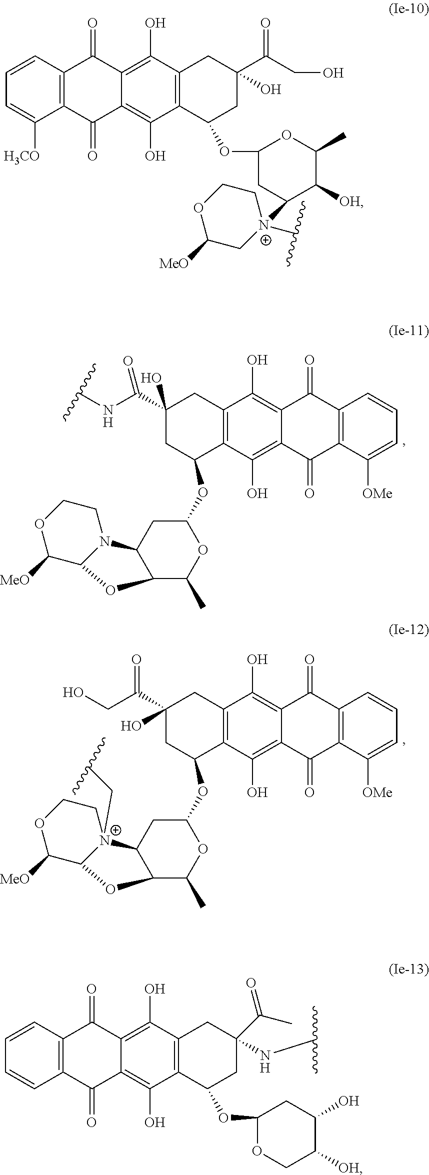
wherein [symbol] is the site that links to L₁ or L₂.
(i) the Vinca alkaloids are selected from vinblastine, vincristine, vindesine_leurosine, vinorelbine, catharanthine, vindoline, vincaminol, vineridine, minovincine, methoxyminovincine, minovincinine, vincadifformine, desoxyvincaminol, vincamajine, vincamine, vinpocetine, and vinburnine and have the following formula: 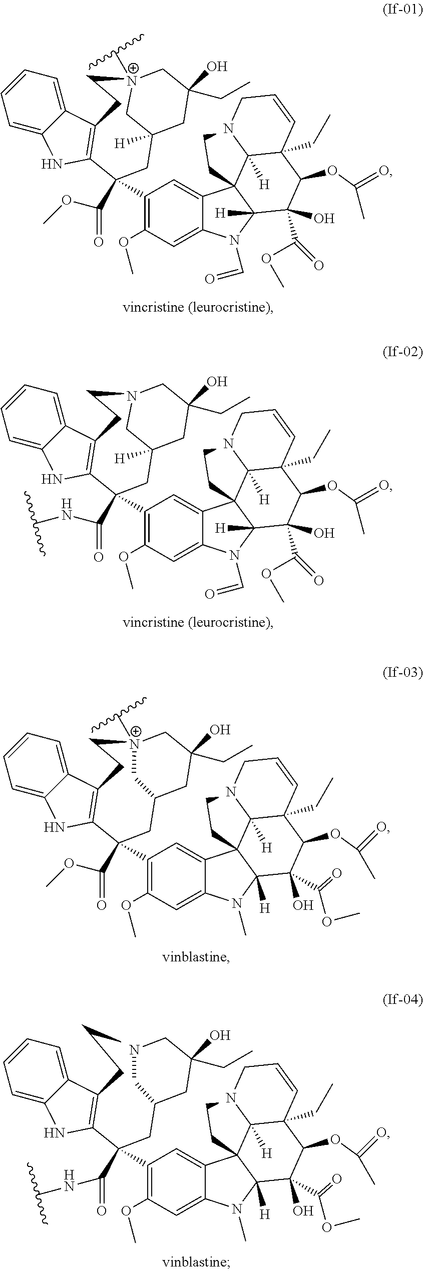 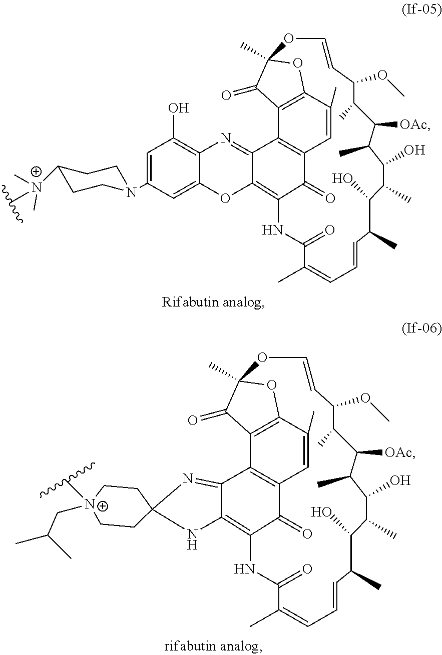
wherein [symbol] is the site linked to L₁ or L₂;
(j) the Dolastatins and their peptidic analogs and derivatives are selected from dolastatin 10, auristatin E (AE), auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), Auristatin F phenylene diamine (AFP) and have the following formula: 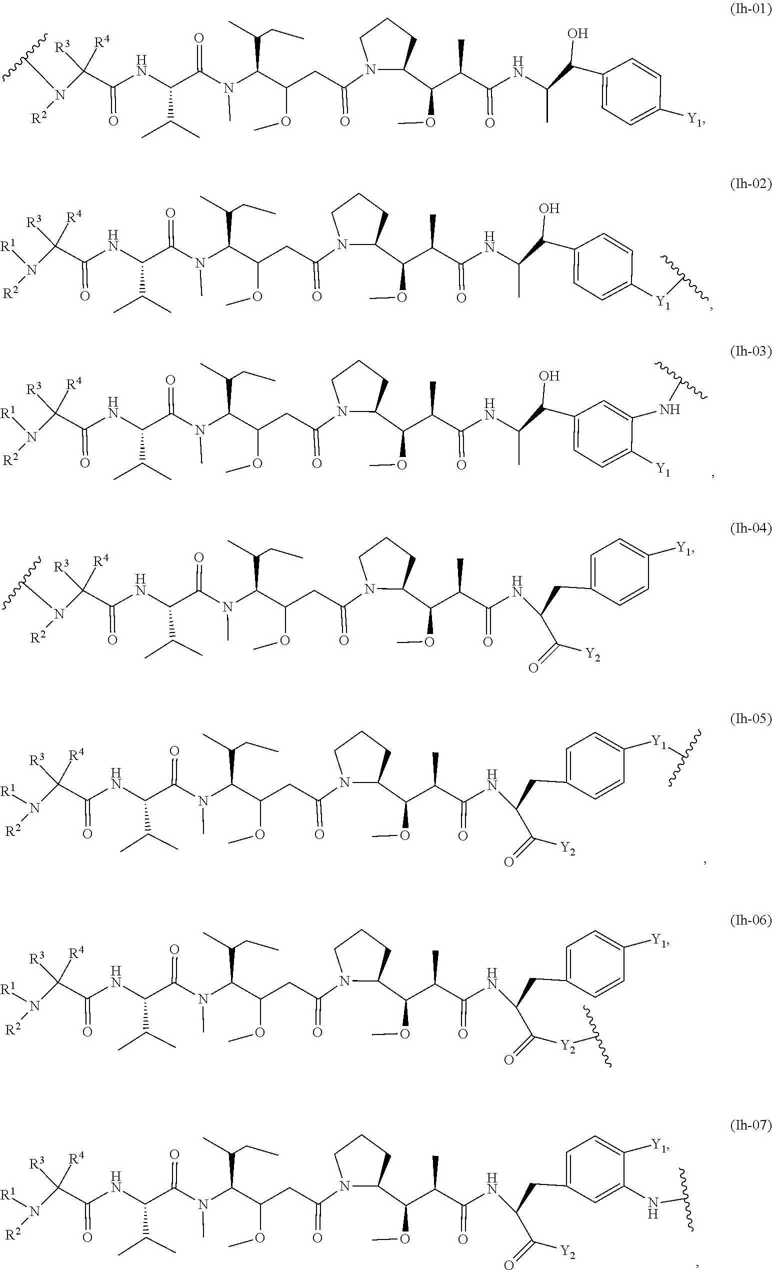 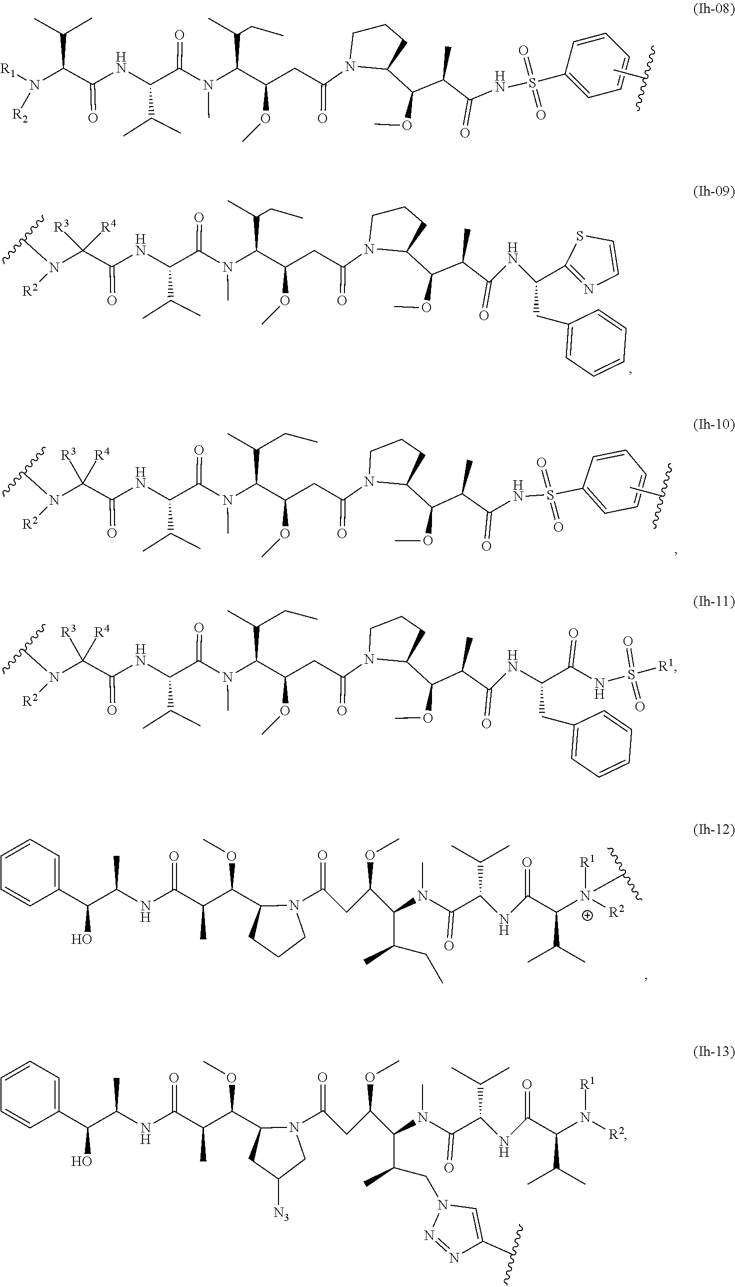 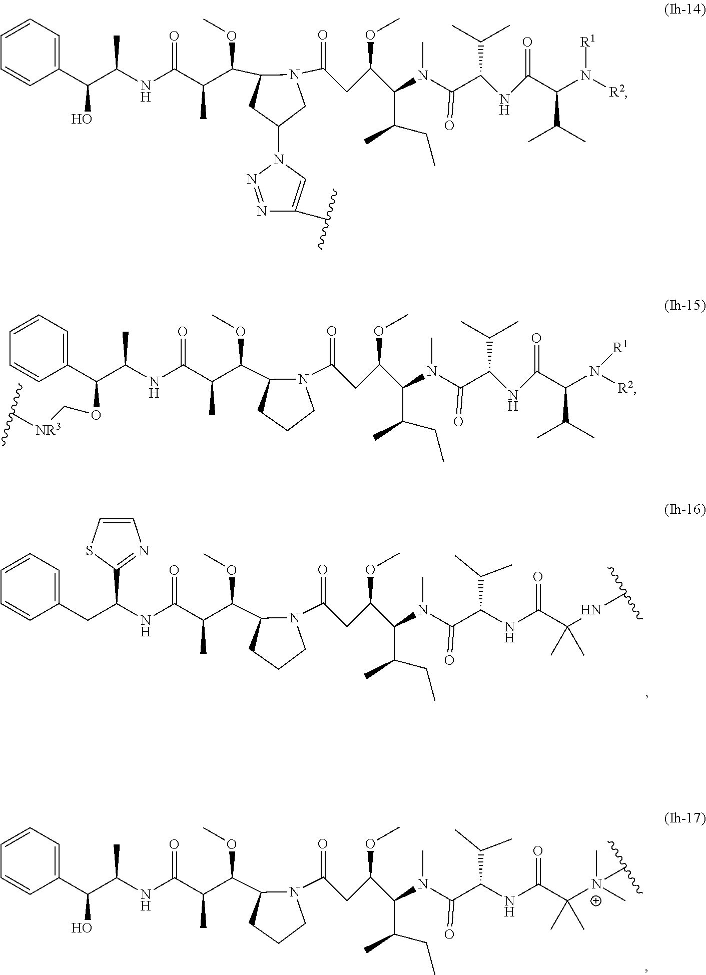
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein R¹, R², R³, R⁴ and R^{S} are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 1000. The two Rs: R¹R₂, R²R₃, R¹R³ or R³R⁴ together can form 3~8-member cyclic ring of alkyl, aryl, heteroaryl, heteroalkyl, or alkylcycloalkyl group;
Y₁ and Y₂ are independently O, NH, NHNH, NR⁸, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ” (that links to L₁ and/or L₂ independently); or OH, NH₂, NHNH₂, NHR⁵, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR¹ when not linked to the connecting site “ [symbol] ”;
R₁₂ is OH, NH₂, NHR¹, NHNH₂, NHNHCOOH, O—R₁—COOH, NH—R₁—COOH, NH—(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR¹R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-500; R₁, R₂, R₃, R₄, R, R′, Z₁, Z₂, and n are defined the same above;
(k) the Hemiasterlin and its analogues have the following formula: 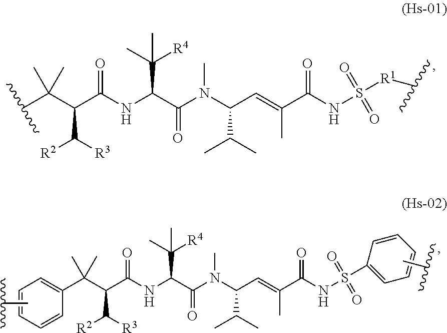
wherein R¹, R², R₃, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; In addition, R²R³ can form 3-8 member cyclic ring of alkyl, aryl, heteroaryl, heteroalkyl, or alkylcycloalkyl group;
(1) the Eribulin has the following formula: 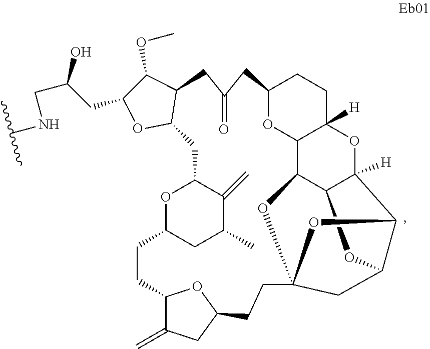 [SMILES: *NC[C@@H](O)C[C@H]1O[C@H]2C[C@H]3O[C@@H](CC[C@@H]4O[C@@H](CC[C@@]56C[C@H]7O[C@@H]8[C@@H](O[C@H]9CC[C@H](CC(=O)C[C@@H]2[C@H]1OC)OC9[C@@H]8O5)[C@H]7O6)CC4=C)C[C@@H](C)C3=C] [symbol]
is a linkage site that links to L₁ and/or L₂ independently;
(m) the Inhibitor of nicotinamide phosphoribosyltransferases (NAMPT) have the following formula, NP₀₁, NP₀₂, NP₀₃, NP₀₄, NP05, NP06, NP₀₇, NP08, and NP_{09:} 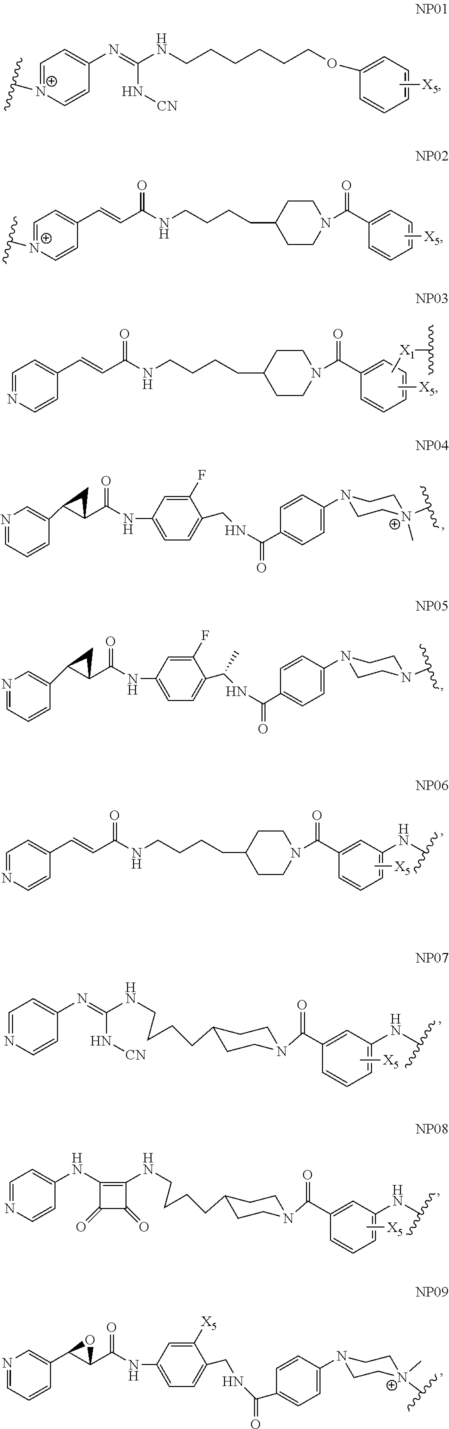
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR¹, OCOR₁, NHCOR₁;
(n) the benzodiazepine dimer and its analogs have the following formulae: 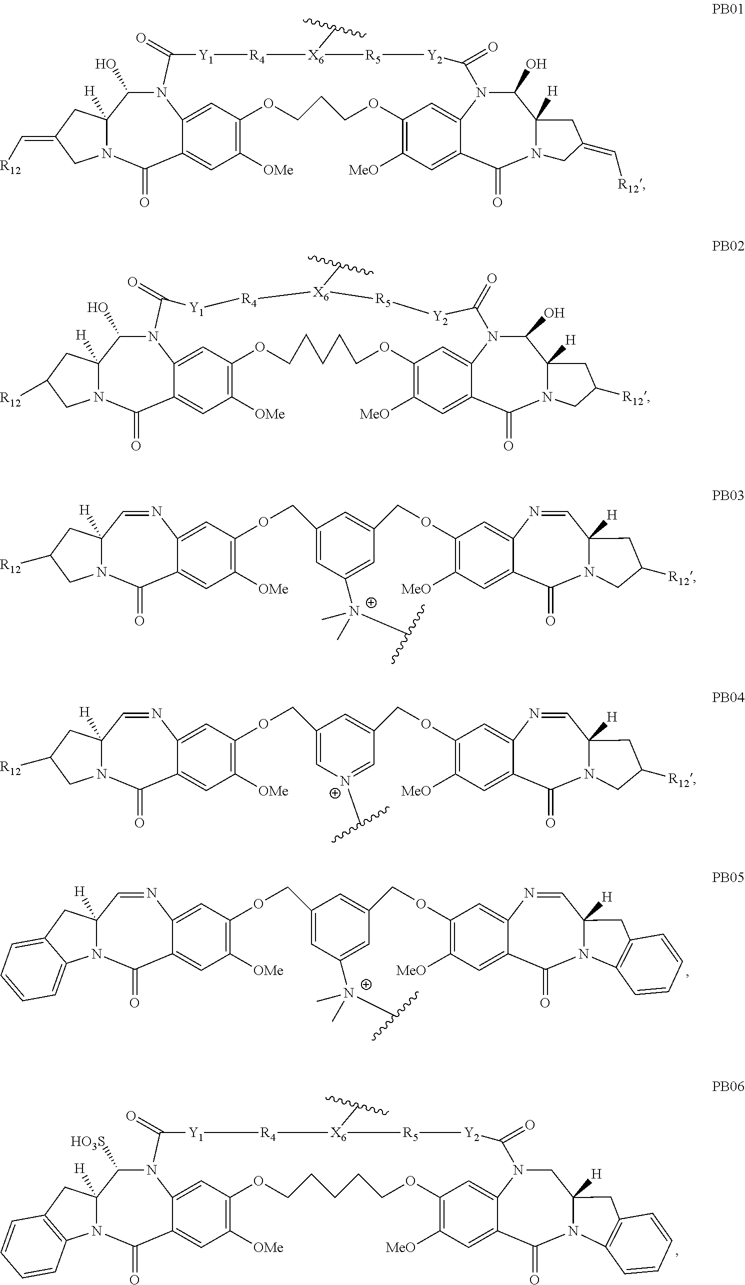 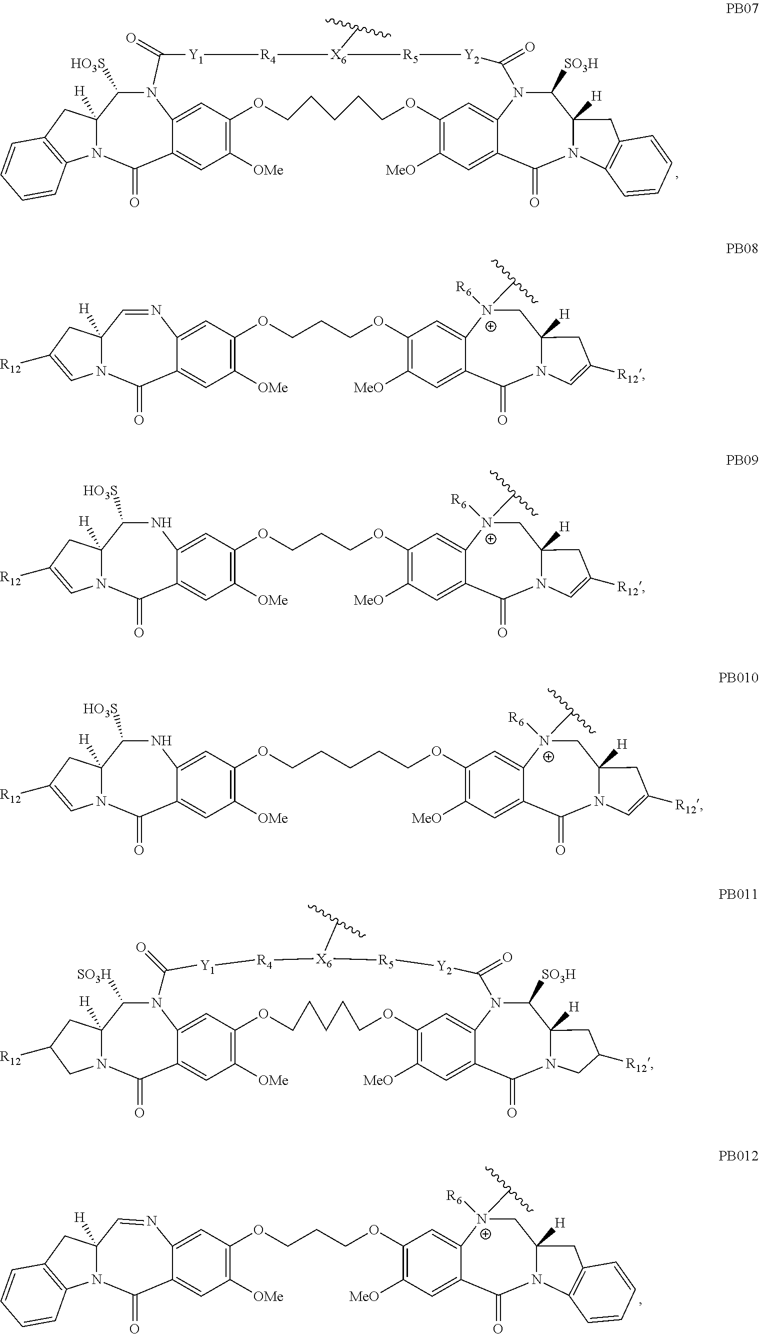 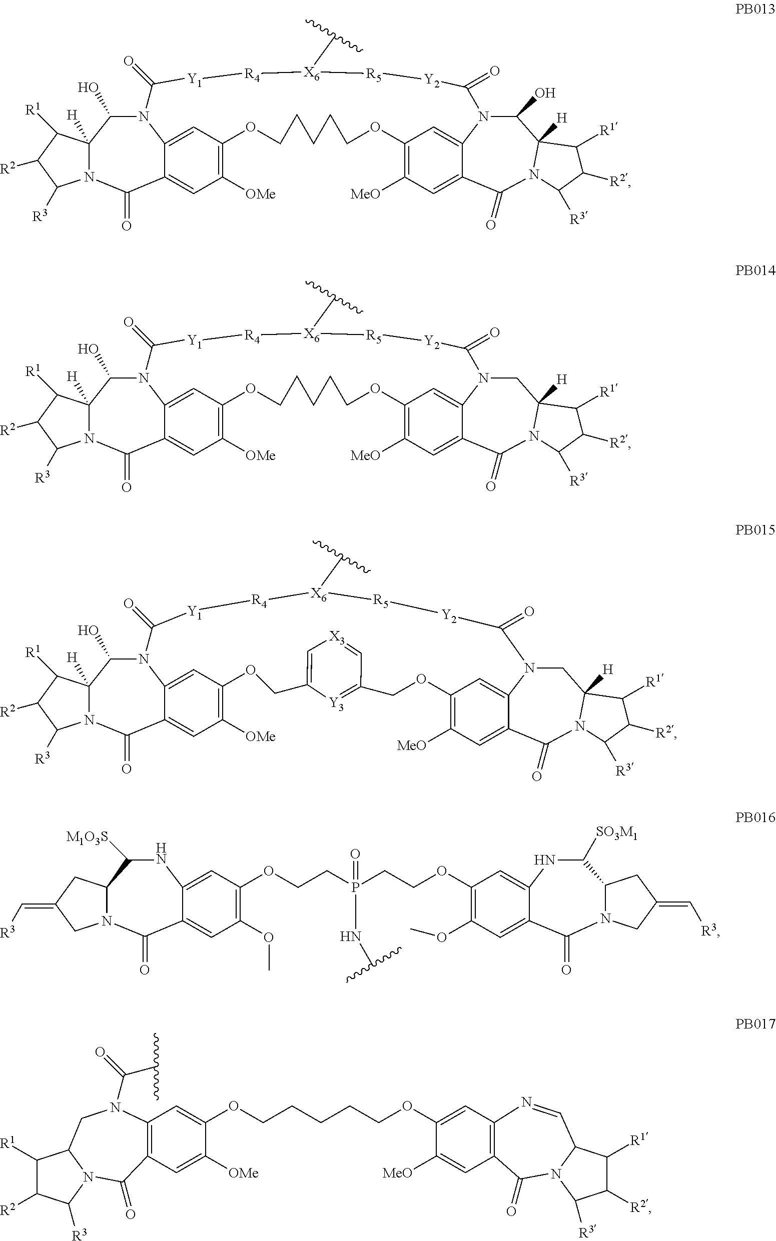 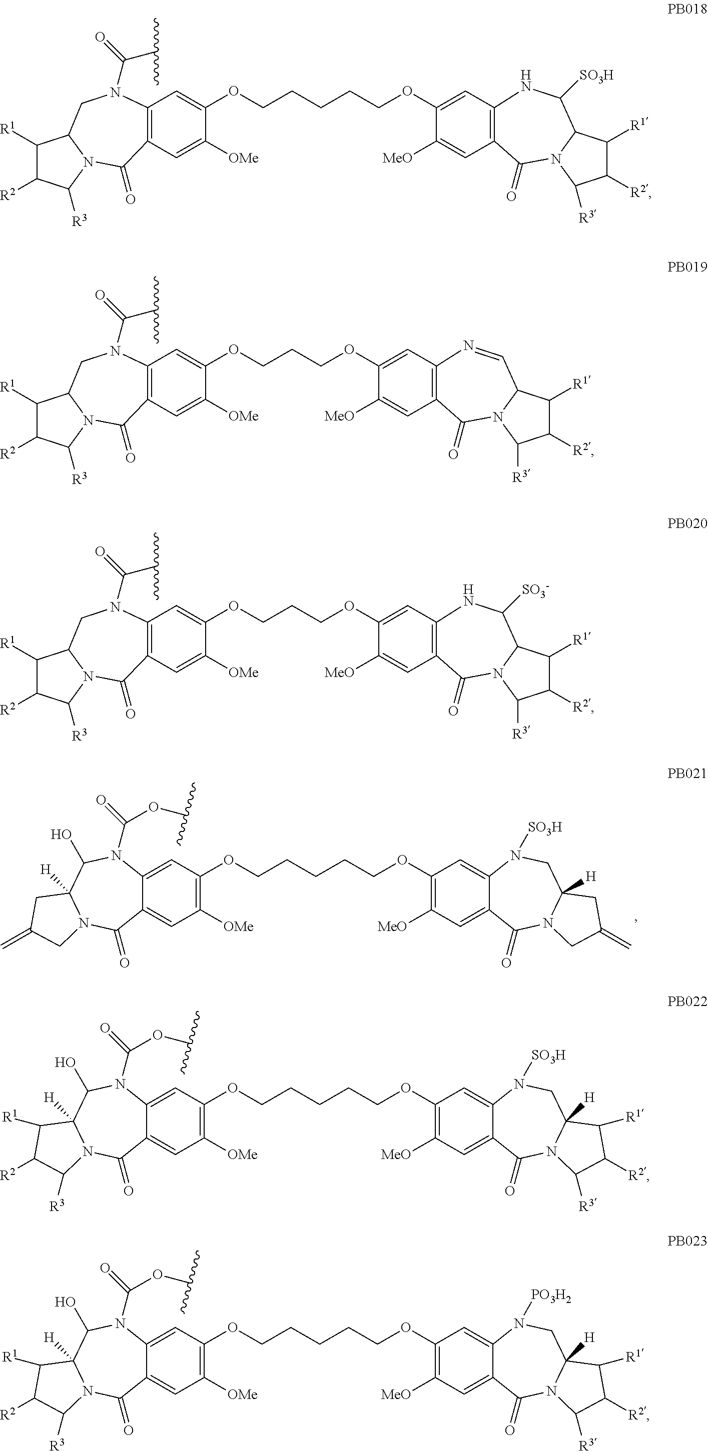 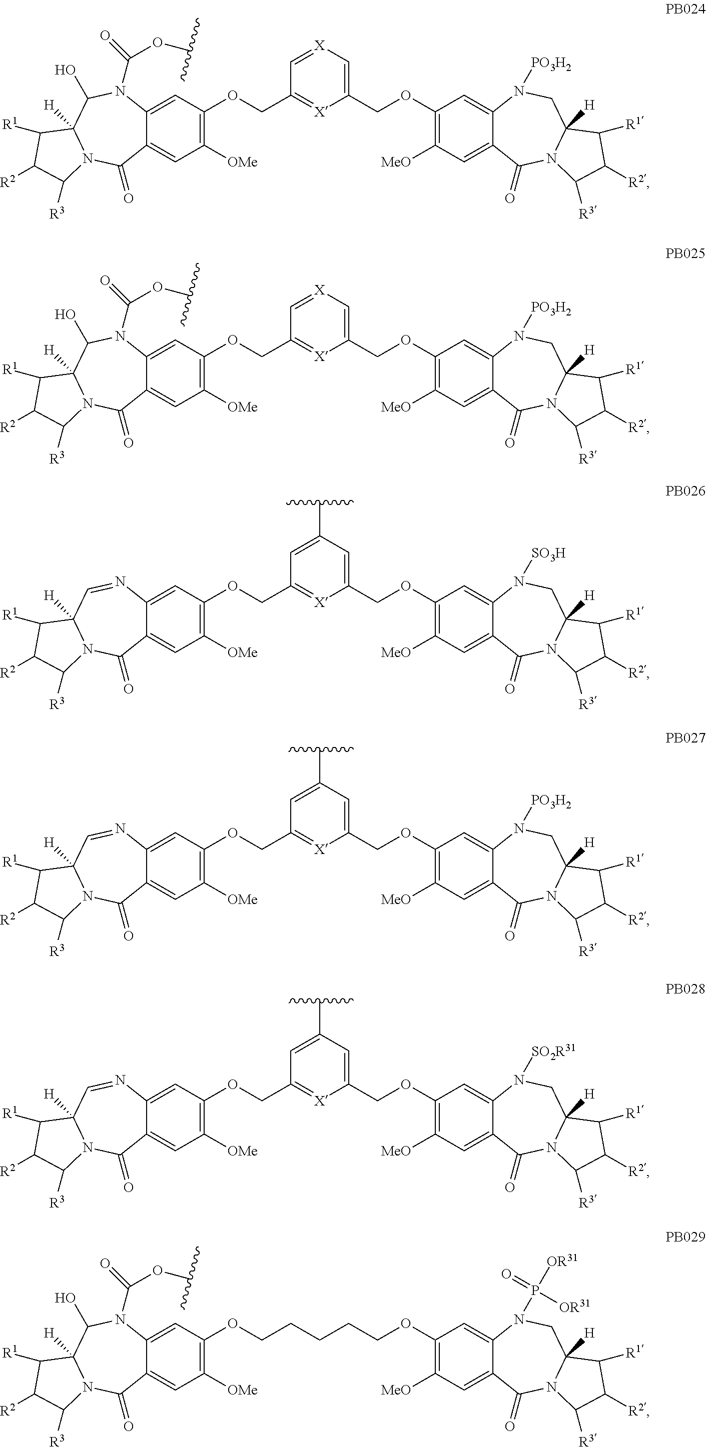 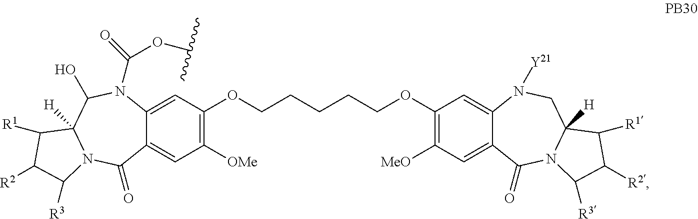
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein, Z₁, Z₂, and n are defined the same above;
X₁, X₂, Y₁ and Y₂ are independently O, N, NH, NHNH, NR⁸, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR^{1;}
R¹, R², R³, R″, R^{2′}, and R^{3′} are independently H; F; C₁; ═O; ═S; ═CH₂; ═CH—R₁, OH; SH; C₁-C₈ linear or branched alkyl, aryl, alkenyl, heteroaryl, heteroalkyl, alkylcycloalkyl, ester (COOR₅ or —OC(O)R⁵), ether (OR₅), amide (CONR⁵), carbamate (OCONR₅), amines (NHR⁵, NR₅R₅′), heterocycloalkyl, or acyloxylamines (—C(O)NHOH, —ONHC(O)R₈); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000. The two Rs: R¹R₂, R²R₃, or R^{2′}R^{3′}, can independently form 3-8-member cyclic ring of alkyl, aryl, heteroaryl, heteroalkyl, or alkylcycloalkyl group;
X₃ and Y₃ are independently N, NH, CH₂ or CR₅, or one of X₃ and Y₃ can be absent;
wherein R₁, and R₂ are C₁-C₈ linear or branched alkyl, heteroalkyl; C₃-C₈ aryl, heteroaryl, alkylcycloalkyl, acyloxyl, alkylaryl, alkylaryloxyl, alkylarylamino, alkylarylthiol; or 1-6 the same or different sequence of amino acid/peptides (Ar)r, r=1-6;
wherein R₄, R₅, R₅′, R⁶, R₁₂ and R₁₂′ are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, acyloxylamines;
Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; Mi and M2 are independently H, Na, K, Ca, Mg, NH₄, NR¹R₂R₃;
X₆ is CH, N, P(O)NH, P(O)NR¹, CHC(O)NH, C₃-C₈ aryl, heteroaryl, alkylcycloalkyl, acyloxyl, alkylaryl, alkylaryloxyl, alkylarylamino, or an Aa (amino acid, is preferably selected from Lys, Phe, Asp, Glu, Ser, Thr, His, Cys, Tyr, Trp, Gln, Asn, Arg);
wherein X and X′ are independently CH₂, or N, and X and/or X′ can be O, S or NH when the six-member aromatic ring that they involved become five-member ring;
Y²¹ is Ms (mesyl), Ts (tosyl), Tf (triflyl), SO₃H, P(O)(OH)₂, CH₂(O)P(O)(OH)₂, glycoside;
R³¹ is H, C₁-C₈ alkyl or Ar, CF₃; “ [symbol] ” is defined the same above
(o) the CC-1065 analog and duocarmycin analogs have the following formula, CC01, CC02, CCO₃, CC04, CC05, CC06 and CC07: 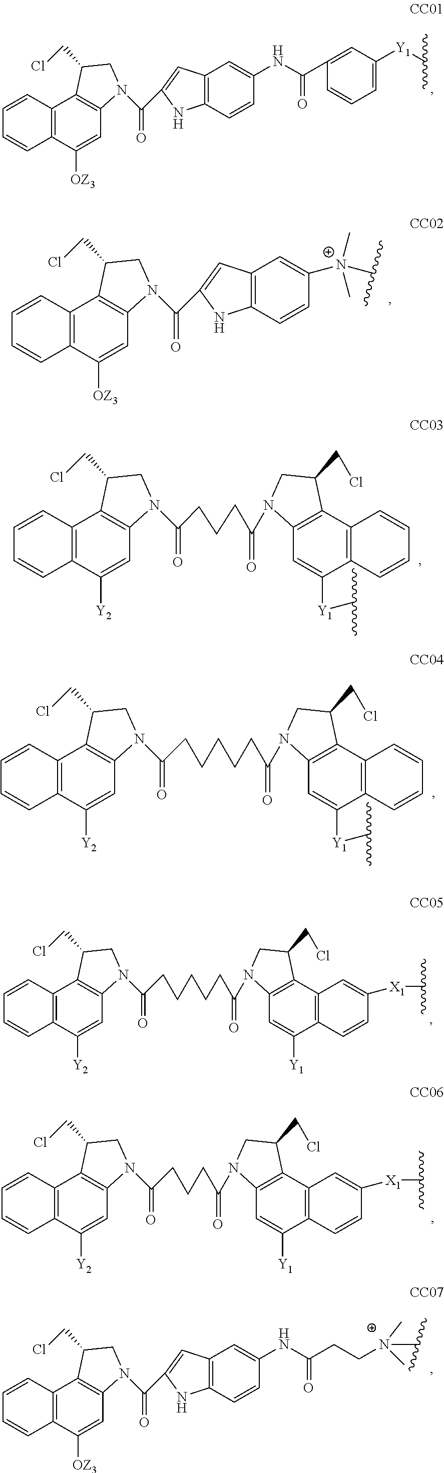
wherein X₁, X₂, Y₁ and Y₂ are independently O, NH, NHNH, NR⁸, S, C(O)O, C(O)NH, OC(O)NH, OC(O)O, NHC(O)NH, NHC(O)S, OCO)N(R₁), N(R₁)C(O)N(R₂), C(O)NHNHC(O) and C(O)NR¹ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M1, M2, and n are defined the same above;
(p) the amatoxin and its analogs have the following formula, Am01, Am02, and Am03: 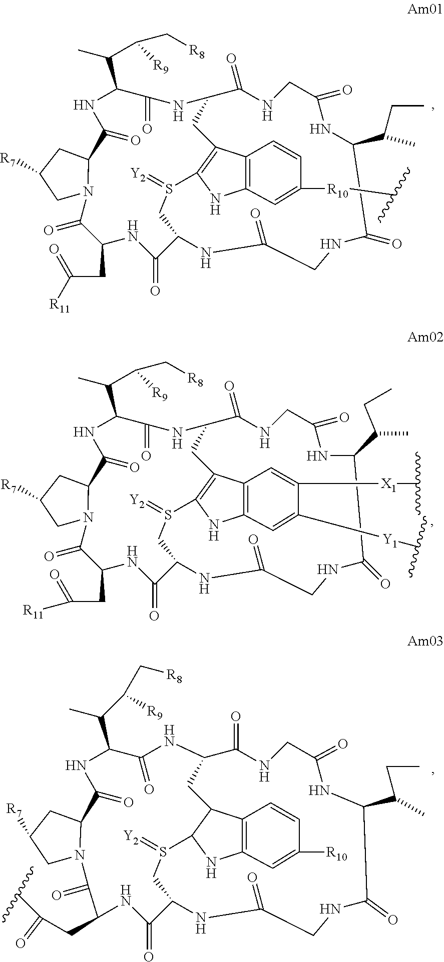
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NR⁵, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NHNHC(O) and C(O)NR₁; R⁷, R⁸, and R⁹ are independently H, OH, OR₁, NH₂, NHR₁, C₁-C₆ alkyl, or absent; Y₂ is O, O₂, NR₁, NH, or absent; Rio is CH₂, O, NH, NR¹,NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH—(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR¹R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁-OPO₃H₂, O(CH₂CH₂O)ₚCH₂CH₂OPO₃H₂, OR₁-NHPO₃H₂, NH—R₁—NHPO₃H₂, or NH(CH₂CH₂O)ₚCH₂—CH₂NHPO₃H₂, wherein (Aa)r is 1-8 aminoacids; n and mi are independently 1-20; p is 1-500; R₁, R₂ and Ar, are the same defined throughout the application; “ [symbol] ” is defined the same above;
(q) the Spliceostatins and pladienolides are spliceostatin A, FR901464, and (2S, 3Z)-5-{[(2R, 3R, 5S, 6S)-6-{(2E, 4E)-5-[(3R, 4R, 5R, 7S)-7-(2-hydrazinyl-2-oxoethyl)—4-hydroxy-1, 6-dioxaspiro[2.5]oct-5-yl]-3-methylpenta-2, 4-dien-1-y-1}-2,5-dimethyltetrahydro-2H-pyran-3-yl]amino}-5-oxopent-3-en-2-yl acetate, Pladienolide B, Pladienolide D, and E7107 having a core structure of Sp-01: 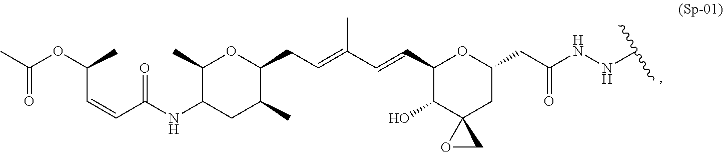 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1]
(r)the protein kinase inhibitors are selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, Vemurafenib, Entrectinib, Palbociclib, Ribociclib, Abemaciclib, Dacomitinib, Neratinib, Rociletinib (CO-1686), Osimertinib, AZD3759, Nazartinib (EGF816), having the following formula, PK01 ~PK41: 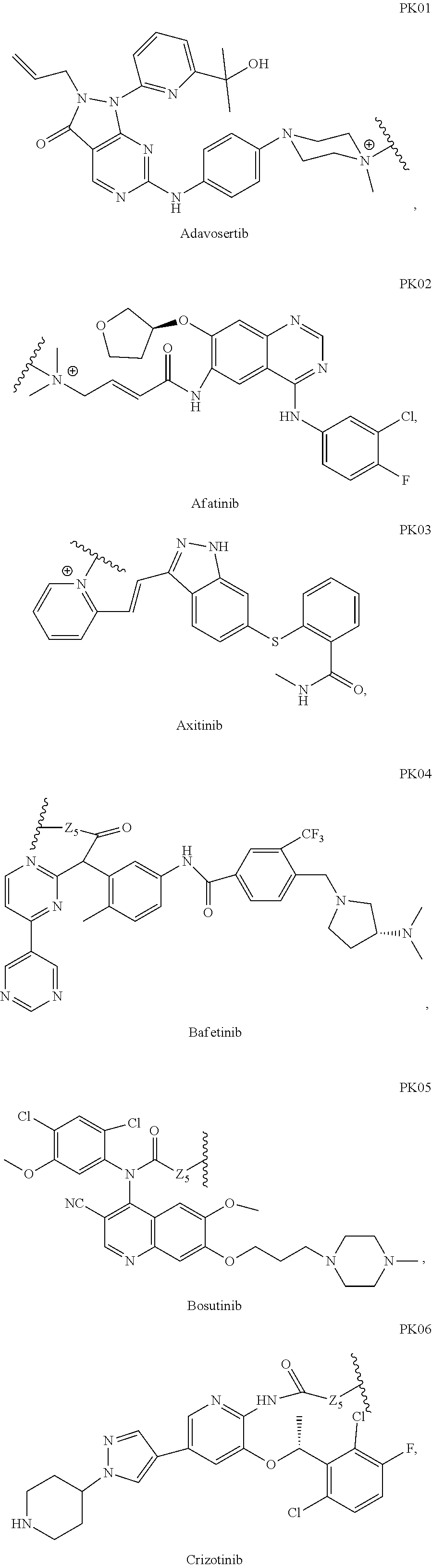 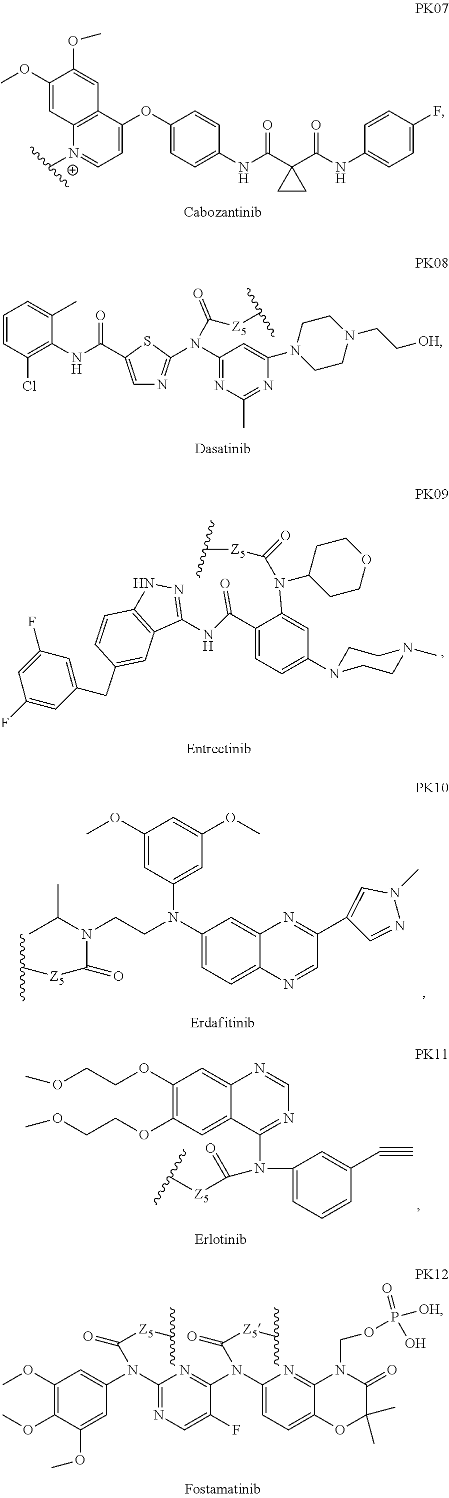 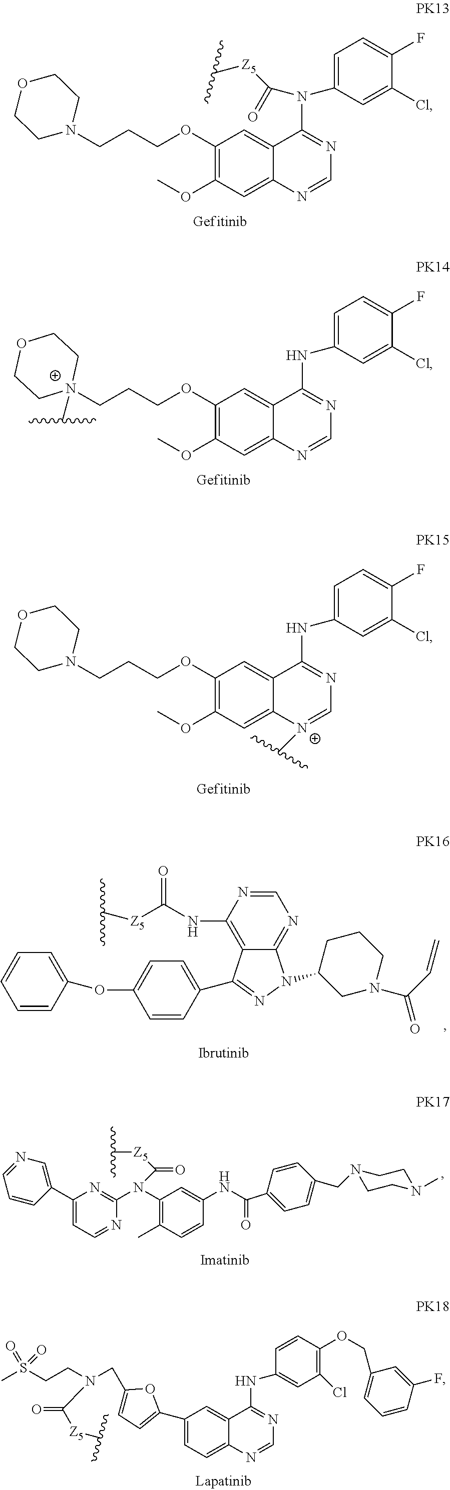 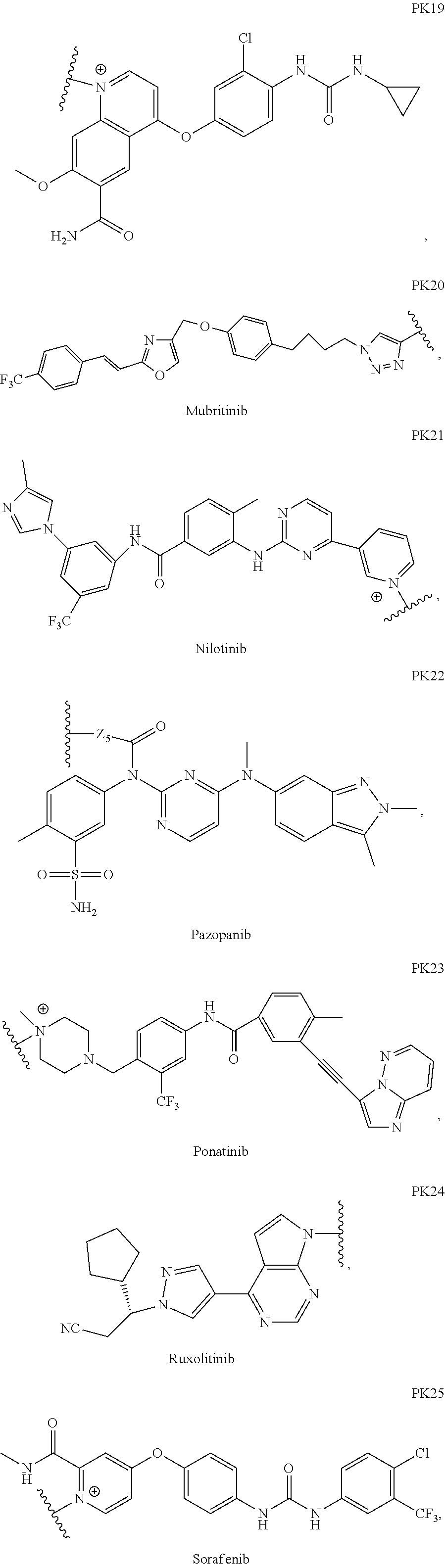 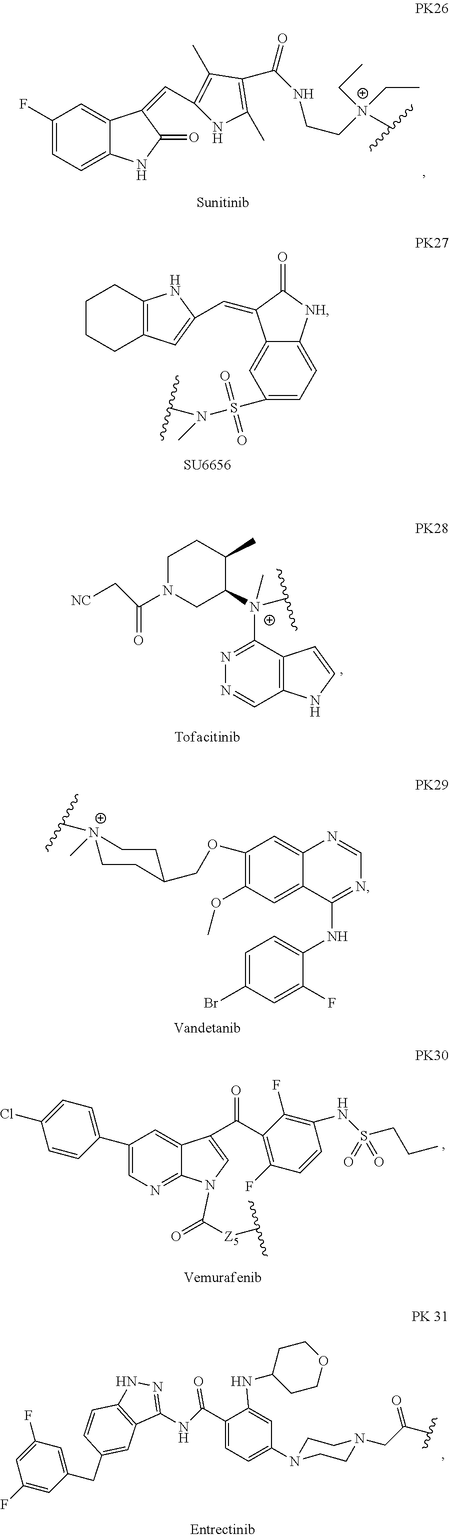 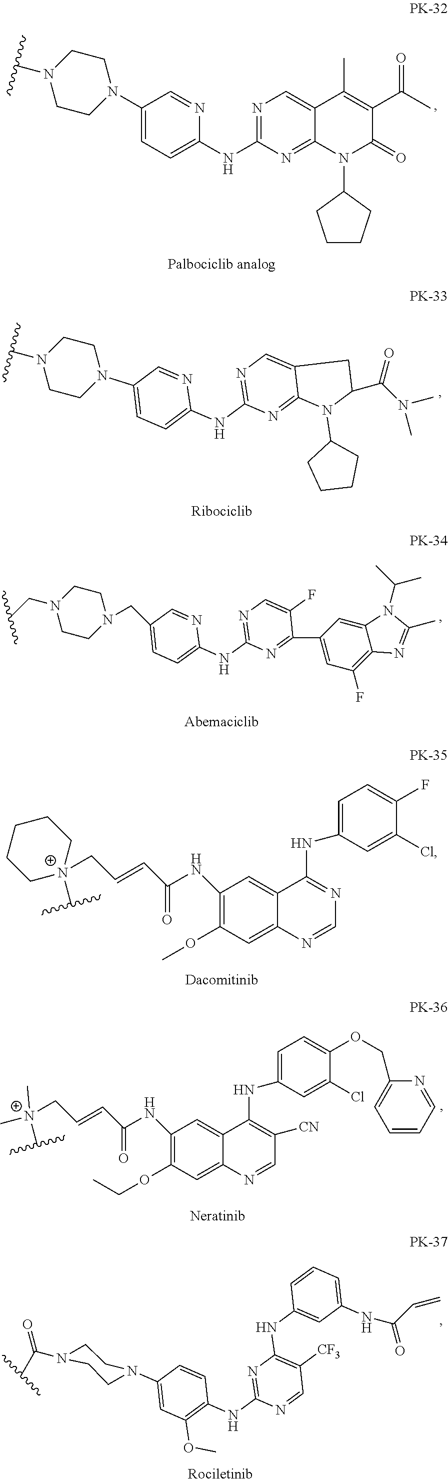 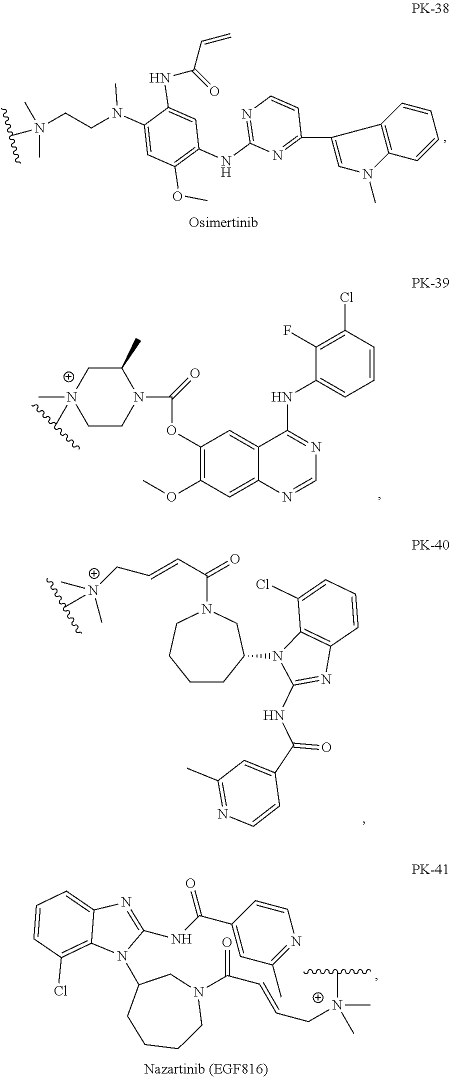
wherein Zs and Zs' are independently selected from O, NH, NHNH, NR⁸, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR^{1;}
(s) the MEK inhibitors are selected from PD0325901, selumetinib (AZD6244), cobimetinib (XL518), refametinib, trametinib (GSK1120212), pimasertib, Binimetinib (MEK162), AZD8330, RO4987655, RO5126766, WX-554, E6201, GDC-0623, PD-325901 and TAK-733, and have the following formula: 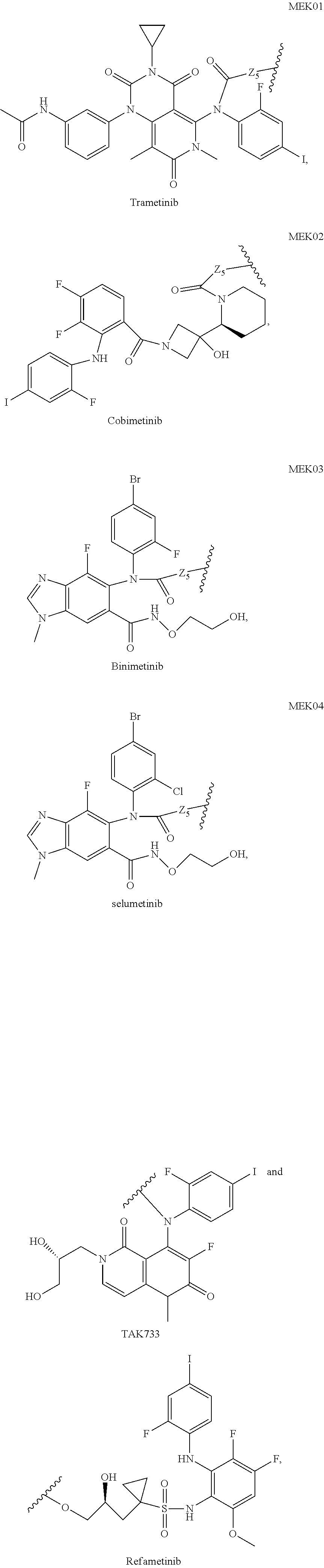
wherein Zs is selected from O, NH, NHNH, NR⁸, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁;
(t) the proteinase inhibitors are selected from Carfilzomib, Clindamycin, Retapamulin, Indibulin, having the following formulae: 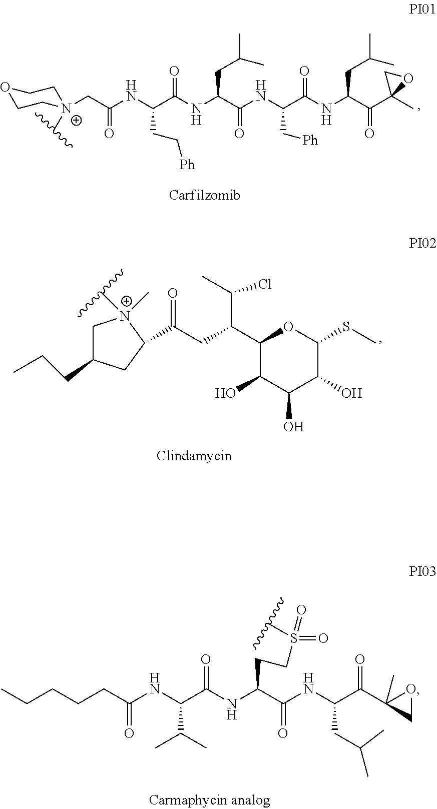
(u) the immunotoxin is selected from Diphtheria toxin (DT), Cholera toxin (CT), Trichosanthin (TCS), Dianthin, Pseudomonas exotoxin A (ETA), Erythrogenic toxins, Diphtheria toxin, AB toxins, Type III exotoxins, proaerolysin, and topsalysin;
(v) the cell receptor agonist or stimulating molecule are selected from: Folate derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (a-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR₁, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones selected from the group consisting of luteinizing hormone-releasing hormone (LHRH) agonists and antagonists, and gonadotropin-releasing hormone (GnRH) agonist selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRR₈), selected from the group consisting of Toll-like receptors' (TLR₈) ligands, C-type lectins and Nodlike Receptors' (NLR₈) ligands; Calcitonin receptor agonists; integrin receptors' and their receptor subtypes' (selected from the group consisting of av3i, αᵥβ₃, αᵥ35, αᵥβ₆, a6p4, a7p1, aL02, aMbp3) agonists (selected from the group consisting of GRGDSPK, cyclo(RGDfV) (Li) and its derives[cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Anticalin (a derivative of Lipocalins); Adnectins (10th FN3 (Fibronectin)); Designed Ankyrin Repeat Proteins (DARPins); Avimers; EGF receptors, or VEGF receptors' agonists;
the cell receptor agonist is selected from the following: LBO1 (Folate), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αvP3 integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR2 for a Toll-like receptor,), LB23 (for an androgen receptor), LB24 (Cilengitide/cyclo(-RGDfV-) for an αᵥ integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) having following structures: 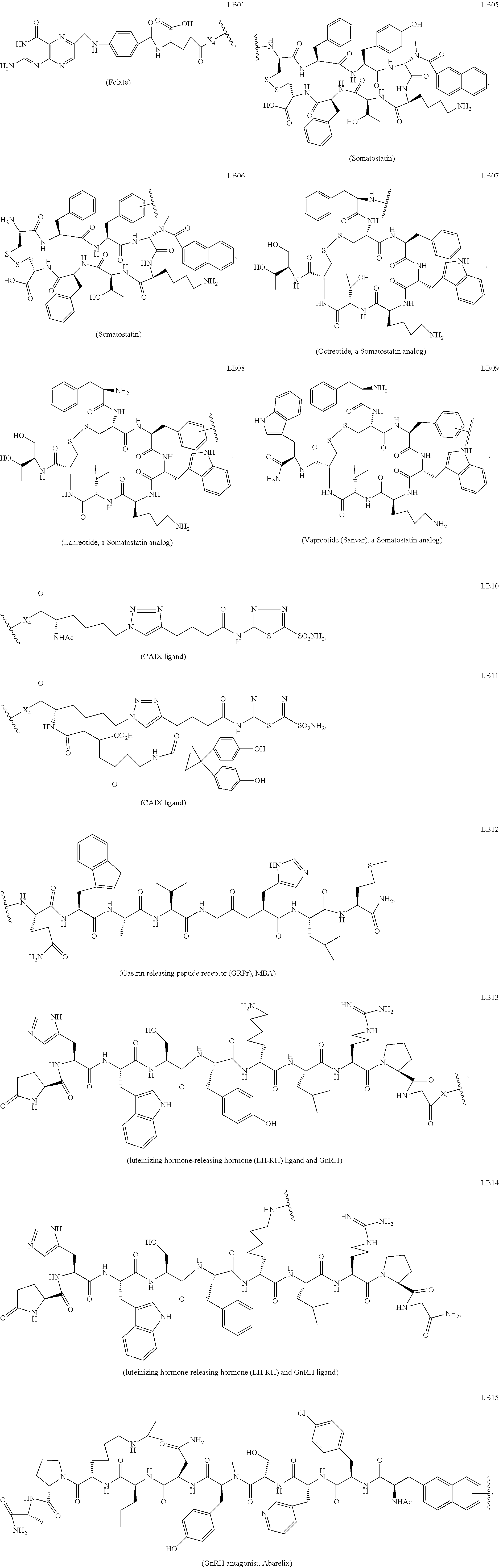 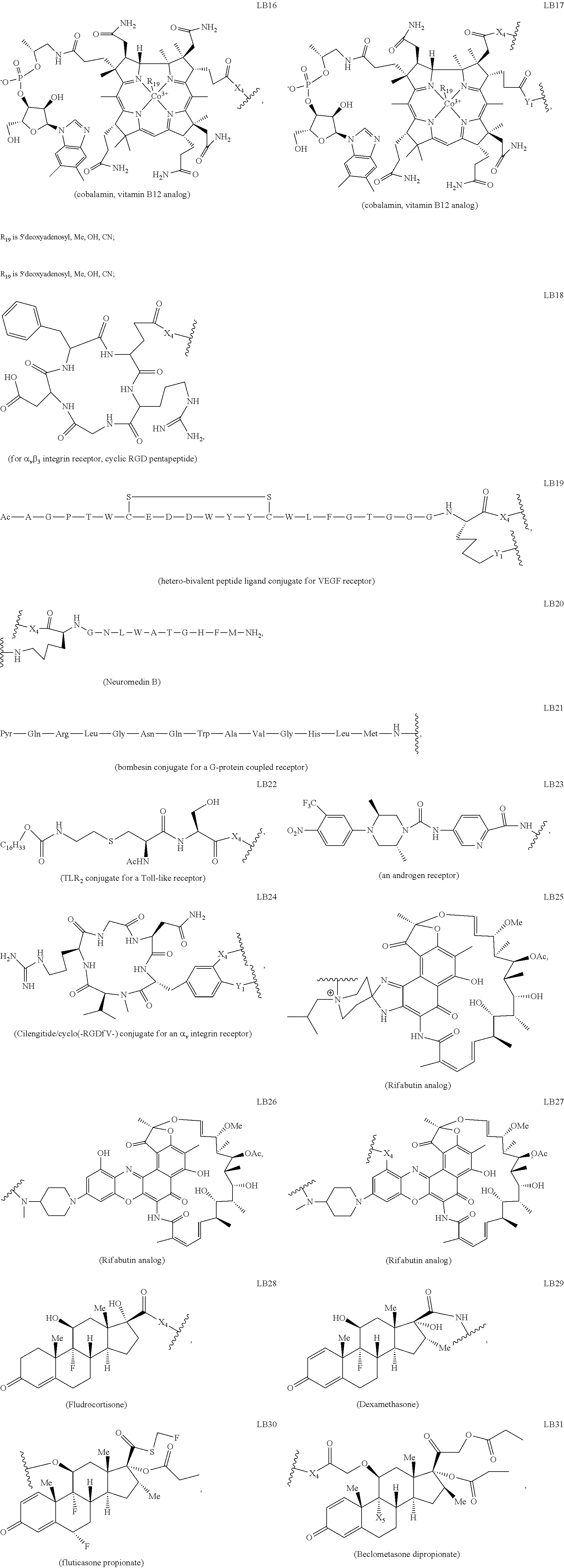 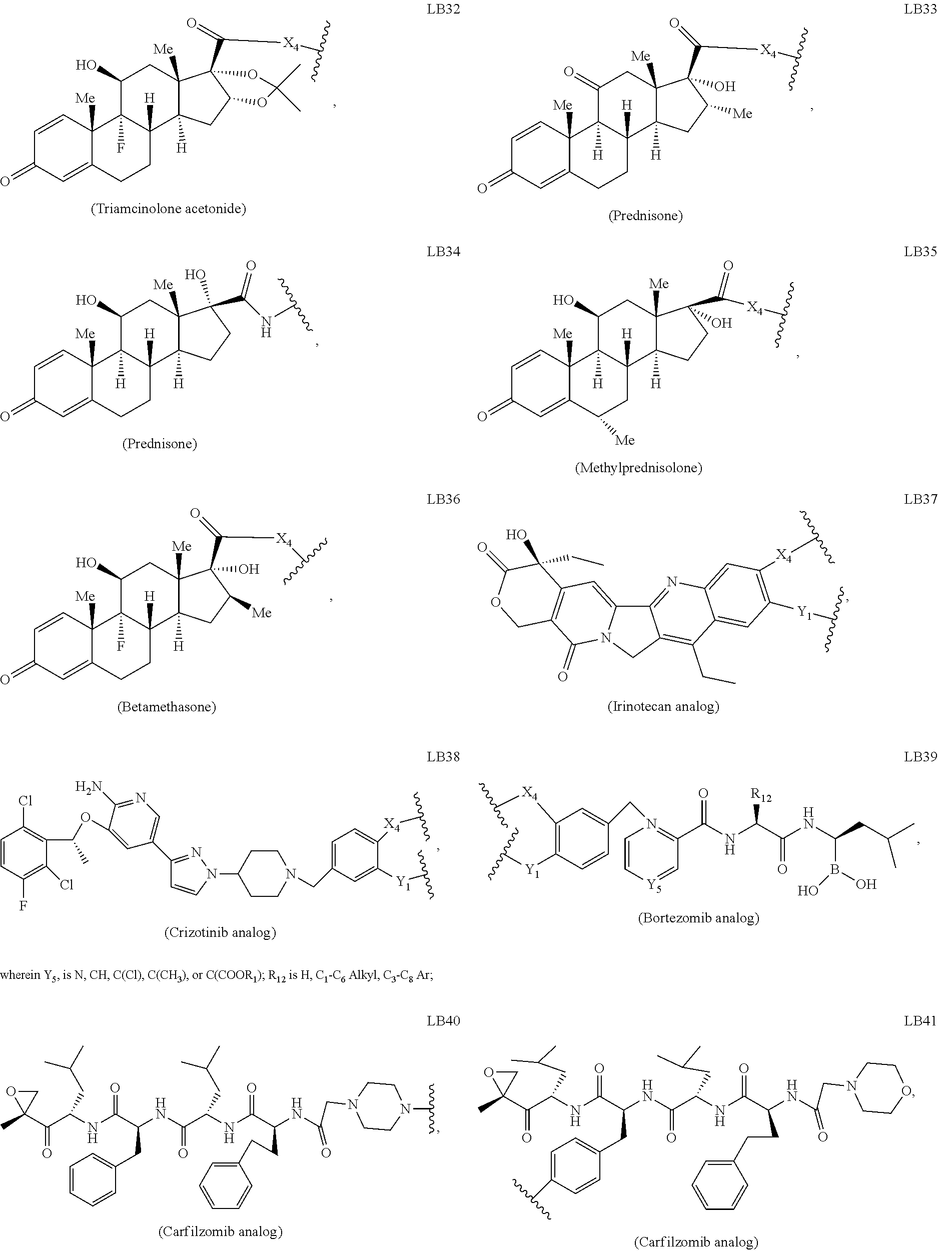 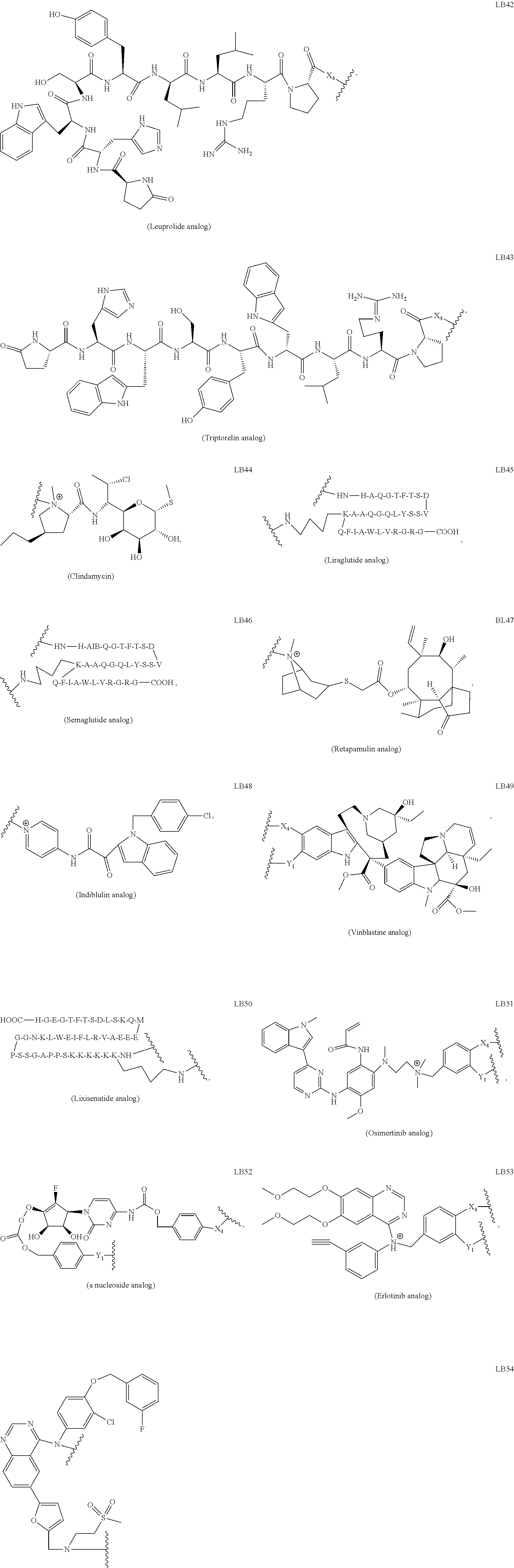
Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁, and R₁ is C₁-C₈ alkyl;
(w) the one, two or more DNA, RNA, mRNA, small interfering RNA (siRNA), microRNA (miRNA), and PIWI interacting RNAs (piRNA) have a structure of: 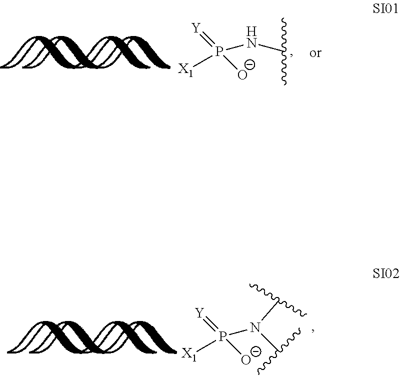
wherein “ [symbol] ” is a site to link the side chain linker; [symbol] is single or double strands of DNA, RNA, mRNA, siRNA, miRNA, or piRNA; X₁, and Y are independently O, NH, NHNH, NR¹, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) or C(O)NR₁.

5. The a linker antibody drug conjugate according to claim 1, wherein the linker component, L₁, L₂, La₁, La₂, Lb₁, Lb₂, Lc₁ and Lc₂, independently contain:
(a). a self-immolative linker component having one of the following structures: 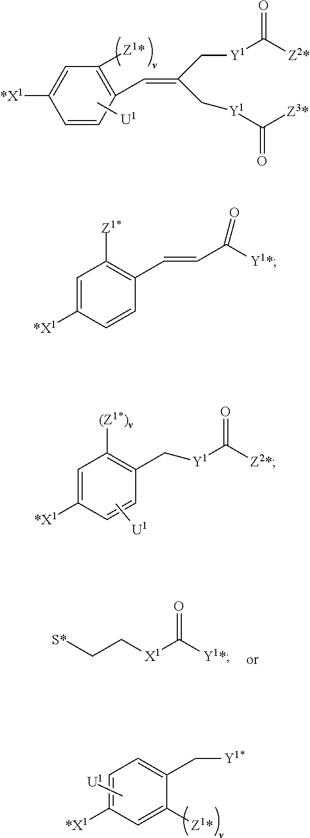
wherein the (*) atom is the point of attachment of additional spacer or releasable linker units, or the cytotoxic agent, and/or the antibody; X¹, Y¹, Z² and Z³ are independently NH, O, or S; Z¹ is independently H, NH, O or S; v is 0 or 1; U¹ is independently H, OH, C₁-C₆ alkyl, (OCH₂CH₂)ₙF, Cl, Br, I, OR₅, SR₅, NR⁵R⁵′, N═NR⁵, N═R⁵, NR⁵R⁵′,NO₂, SOR₅R⁵′, SO₂R⁵, S O₃R⁵, OS O₃R⁵, PR5R⁵′, POR₅R⁵′, PO₂R₅R₅′, OPO(OR₅)(OR₅′), or OCH2PO(OR₅(OR₅′) wherein R⁵ and R⁵′ are as defined above; preferably R⁵ and R⁵′ are independently selected from H, C₁-C₈alkyl; C₂-C₅ alkenyl, alkynyl, heteroalkyl; C₃~C₈ aryl, heterocyclic, carbocyclic, cycloalkyl, heterocycloalkyl, heteroaralkyl, alkylcarbonyl; or pharmaceutical cation salts;
(b). a non-self-immolative linker component having one of the following structures: 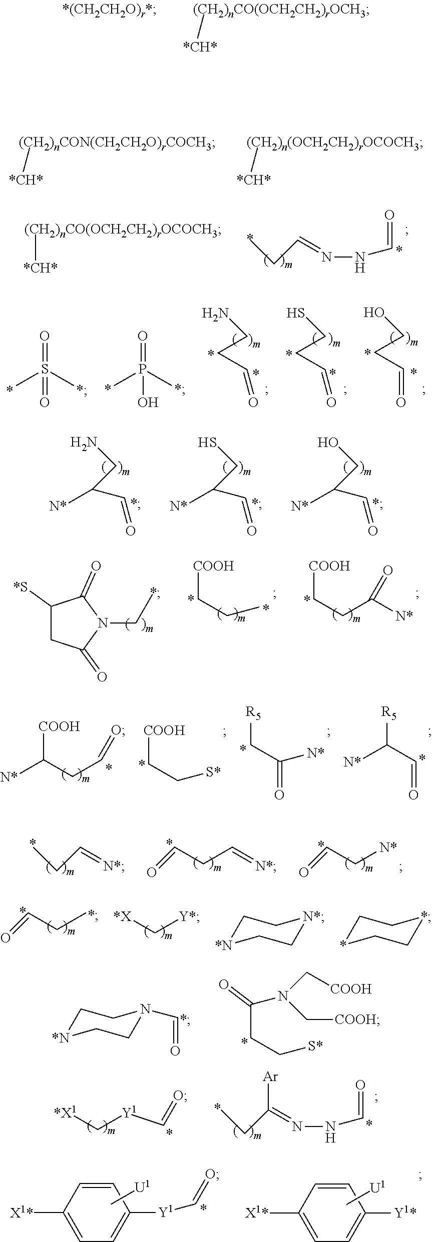 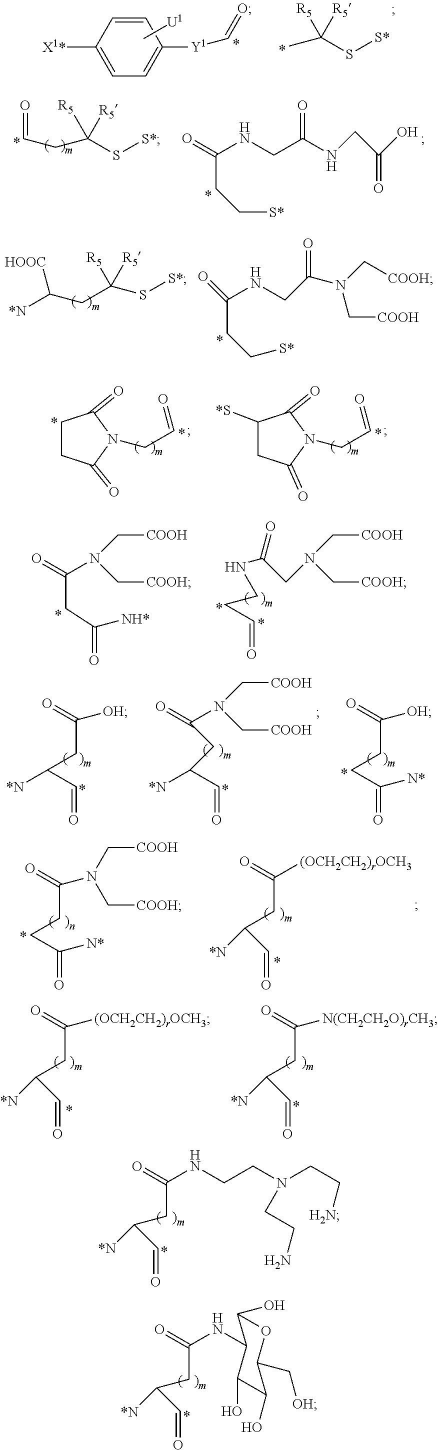 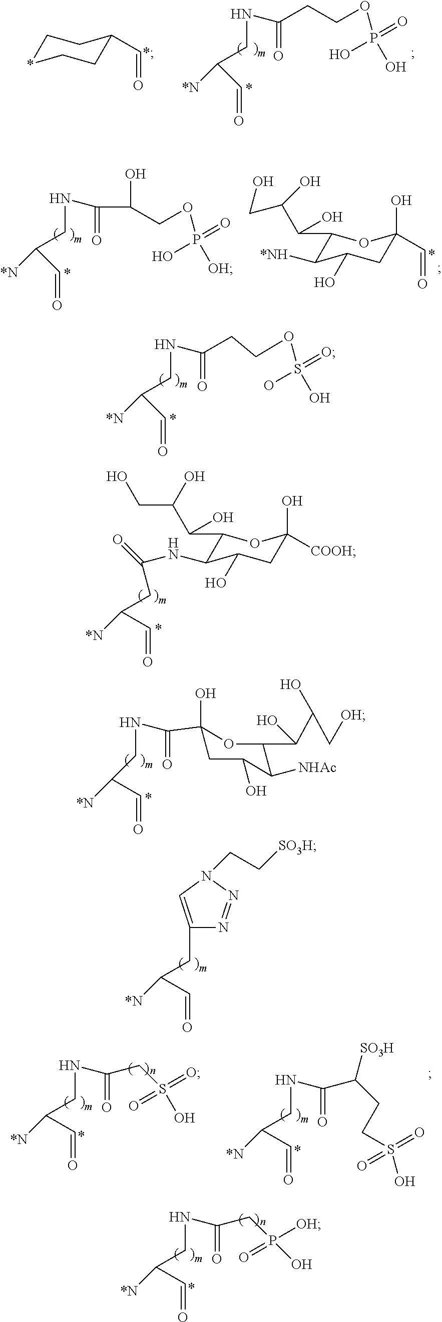
wherein the (*) atom is the point of attachment of additional spacer R₁ or releasable linkers, the cytotoxic agents, and/or the binding molecules; X¹, Y¹, U^{l}, R₁, R⁸, R₅' are defined as above; r is 0~100; m and n are 0-6 independently;
(c). one or more linker components of 6-maleimidocaproyl (“MC”), maleimidopropanoyl (“MP”), valine-citrulline (“val-cit” or “vc”), alanine-phenylalanine (“ala-phe” or “af”), p-amino-benzyloxycarbonyl (“PAB”), 4-thiopentanoate (“SPP”), 4-(N-maleimidomethyl)-cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1-8 natural or unnatural amino acid unites, wherein lysine, glutamic acid, aspartic acid, cysteine, or tyrosine may contain a side chain polyethyleneoxy unit of formula (OCH₂CH₂)ₚOR₃, (OCH₂CH(CH₃))ₚOR₃, NH(CH₂CH₂O)ₚR₃, C(O)(CH₂CH₂O)ₚR₃, C(O)(CH₂CH₂OCH₂CH)ₚR₃,NH(CH₂CH(CH₃)O)ₚR₃, C(O)(CH₂OCH₂CH)ₚR₃, N[(CH₂CH₂O)ₚR₃][(CH₂CH₂O)ₚ—R₃-], (OCH₂CH₂)ₚCOOR₃, or CH₂CH₂(OCH₂CH₂)ₚCOOR₃, NH(CH₂CH₂O)ₚCH₂CH₂R₃, wherein p and p′ are independently an integer selected from 0 to about 100, or combination thereof; wherein R₃ and R₃-are independently H, OH, NH₂, N(CH₂)2, N(C₂H₅)₂, N(C₃H₇), C(═O)H, C(═O)CH₃, C₁-C₈ of alkyl, NH(C₄~C₇ glycoside) or NH(C₄~C₇ glycoside)₂; or combination above thereof;
(d). one or more releasable linker component, having the structure of the following: —(CR₅R⁶)ₘ(Aa)ᵣ(CR₇R₈)ₙ(OCH₂CH₂)ₜ—, —(CR₅R⁶)ₘ(CR₇R₈)ₙ(Aa)ᵣ(OCH₂CH₂)ₜ—, —(Aa)ᵣ(CR₅R⁶)ₘ—(CR₇R₈)ₙ(OCH₂CH₂)ₜ—, —(CR₅R⁶)ₘ(CR₇R₈)ₙ(OCH₂CH₂)ᵣ(Aa)ₜ—, —(CR₅R⁶)ₘ(CR₇═CR₈)(CR₉R₁₀)ₙ—(Aa)ₜ—(OCH₂CH₂)ᵣ, —(CR₅R⁶)ₘ(NR₁₁CO)(Aa)ₜ(CR₉R₁₀)ₙ—(OCH₂CH₂)ᵣ—, —(CR₅R⁶)ₘ(Aa)ₜ—(NR₁₁CO)(CR₉R₁₀)ₙ—(OCH₂CH₂)ᵣ, —(CR₅R⁶)ₘ(OCO)(Aa)ₜ(CR₉R₁₀)ₙ—(OCH₂C₁₂)ᵣ, —(CR₈R₆)ₘ—(OCNR⁷)(Aa)ₜ(CR₉R₁₀)ₙ—(OCH₂CH₂)ᵣ—, —(CR₅R⁶)ₘ(CO)(Aa)ₜ—(CR₉R₁₀)ₙ(OCH₂CH₂)ᵣ—, —(CR₅R⁶)ₘ—(NR¹¹CO)(Aa)ₜ(CR₉R₁₀)ₙ—(OCH₂CH₂)ᵣ, —(CR₅R⁶)ₘ—(OCO)(Aa)ₜ(CR₉R₁₀)ₙ—(OCH₂CH₂)ᵣ, —(CR₅R⁶)ₘ(OCNR⁷)(Aa)ₜ(CR₉R₁₀)ₙ—(OCH₂CH₂)ᵣ, —(CR₅R⁶)ₘ(CO)(Aa)ₜ(CR₉R₁₀)ₙ—(OCH₂CH₂)ᵣ—, —(CR₅R⁶)ₘ-phenyl-CO(Aa)ₜ(CR₇R₈)ₙ—, —(CR₅R⁶)ₘ-furyl-CO(Aa)ₜ(CR₇R₈)ₙ—, —(CR₅R⁶)ₘ-oxazolyl-CO(Aa)ₜ(CR₇R₈)ₙ—, —(CR₅R⁶)mthiazolyl-CO-(Aa)ₜ(CCR₇R₈)ₙ—, —(CR₅R⁶)ₜ-thienyl-CO—(CR₇R₈)ₙ—, —(CR₅R⁶)ₜ-imidazolyl-CO—(CR₇R₈)ₙ—, —(CR₅R⁶)ₜ—morpholino-CO(Aa)ₜ—(CR₇R₈)ₙ—, —(CR₅R⁶)tpiperazino-CO(Aa)ₜ(CR₇R₈)ₙ—, —(CR₅R⁶)ₜ-N-methyl-piperazin-CO(Aa)ₜ—(CR₇R₈)ₙ—, —(CR₅R)ₘ-(Aa)tphenyl-, —(CR₅R⁶)ₘ-(Aa)tfuryl-, —(CR₅R⁶)ₘ-oxazolyl(Aa)ₜ—, —(CR₈R₆)ₘ-thiazolyl(Aa)ₜ—, —(CR₅R⁶)ₘ-thienyl-(Aa)ₜ—, —(CR₅R⁶)ₘ-imidazolyl(Aa)ₜ—, —(CR₅R₆)ₘ-morpholino-(Aa)ₜ—, —(CR₅R⁶)ₘ-piperazino-(Aa)ₜ—, —(CR₅R⁶)ₘN-methylpiperazino-(Aa)ₜ—, —K(CR₅R⁶)ₘ(Aa)ᵣ(CR₇R₈)ₙ(OCH₂CH₂)ₜ—, —K(CR₅R⁶)ₘ(CR₇R₈)ₙ—(Aa)ᵣ(OCH₂CH₂)ₜ—, —K(Aa)ᵣ (CR₅R⁶)ₘ(CR₇R₈)ₙ(OCH₂CH₂)ₜ—, —K(CR₅R⁶)ₘ(CR₇R₈)ₙ—(OCH₂CH₂)ᵣ(Aa)ₜ—, —K(CR₅R)ₘ—(CR₇═CR₈)(CR₉R₁₀)ₙ(Aa)ₜ(OCH₂CH₂)ᵣ—, —K(CR₅R⁶)ₘ(NR¹¹CO)(Aa)ₜ(CR₉R₁₀)ₙ(OC₁₁₂C₁₁₂)ᵣ—, —K(CR₅R⁶)ₘ(Aa)ₜ(NR¹¹CO)—(CR₉R₁₀)ₙ(OCH₂—CH₂)ᵣ, —K(CR₅R⁶)ₘ(OCO)(Aa)ₜ(CR₉R₁₀)ₙ—(OCH₂CH₂)ᵣ, —K(CR₅R⁶)ₘ(OCNR⁷)(Aa)ₜ—(CR₉R₁₀)ₙ—(OCH₂CH₂)ᵣ, —K(CR₅R⁶)ₘ(CO)(Aa)ₜ—(CR₉R₁₀)ₙ(OCH₂CH₂)ᵣ —K(CR₅R⁶)ₘ(NR¹¹CO)—(Aa)ₜ(CR₉R₁₀)ₙ(OCH₂CH₂)ᵣ—, —K(CR₅R⁶)ₘ—(OCO)(Aa)ₜ(CR₉R₁₀)ₙ(OCH₂CH₂)ᵣ-—K(CR₅R⁶)ₘ(OCNR⁷)(Aa)ₜ(CR₉R₁₀)ₙ(OCH₂CH₂)ᵣ—, —K(CR₅R⁶)ₘ(CO)(Aa)ₜ(CR₉R₁₀)ₙ—(OCH₂CH₂)ᵣ-—K(CR₈R₆)ₘ-phenyl-CO—(Aa)ₜ(CR₇R₈)ₙ—, -K-(CR₅R⁶)ₘ-furyl-CO(Aa)ₜ—(CR₇R₈)ₙ—, —K(CR₅R⁶)ₘ-oxazolyl-CO(Aa)ₜ(CR₇R₈)ₙ—, —K(CR₈R₆)ₘ-thiazolyl-CO(Aa)ₜ—(CR₇R₈)ₙ—, —K(CR₅R⁶)ₜthienyl-CO(CR₇R₈)ₙ—, —K(CR₅R⁶)ₜimidazolylCO(CR₇R₈)ₙ—, —K(CR₅R⁶)tmorpholino-CO(Aa)ₜ(CR₇R₈)ₙ—, —K(CR₅R⁶)tpiperazino-CO(Aa)ₜ—(CR₇R₈)ₙ—, —K(CR₅R⁶)ₜ-N-methylpiperazinCO(Aa)ₜ(CR₇R₈)ₙ—, —K(CR₅R)ₘ(Aa)tphenyl, —K—(CR₈R₆)ₘ-(Aa)ₜfuryl-, —K(CR₈R₆)ₘ-oxazolyl(Aa)ₜ—, —K(CR₅R⁶)ₘ-thiazolyl(Aa)ₜ—, —K(CR₈R₆)ₘ-thienyl-(Aa)ₜ—, —K(CR₅R⁶)ₘ-imidazolyl(Aa)ₜ—, —K(CRR₆)ₘmorpholino(Aa)ₜ—, —K(CR₅R⁶)ₘpiperazino(Aa)ₜ, —K(CR₅R⁶)ₘN-methylpiperazino-(Aa)ₜ—; wherein m, n, R₃, R₄, and R⁵ are described above; Aa is an amino acid, t and r are 0-100 independently; R⁶, R⁷, and R₅ are independently chosen from H; halide; C₁-C₈ of alkyl, aryl, alkenyl, alkynyl, ether, ester, amine or amide, which optionally substituted by one or more halide, CN, NR¹R₂, CF₃, OR₁, Aryl, heterocycle, S(O)R₁, SO₂R₁, —CO₂H, —SO₃H, —OR₁, —CO₂R₁, —CONR¹, —PO₂R₁R₂, —PO₃H or P(O)R₁R₂R₃; K is NR¹, —SS—, —C(═O)—, —C(═O)NH—, —C(═O)O——C═NH—O—, —C═N—NH—, —C(═O)NH—NH—, O, S, Se, B or C₃-C₆ heteroaromatic group;
(e). one or several of the following structure units: 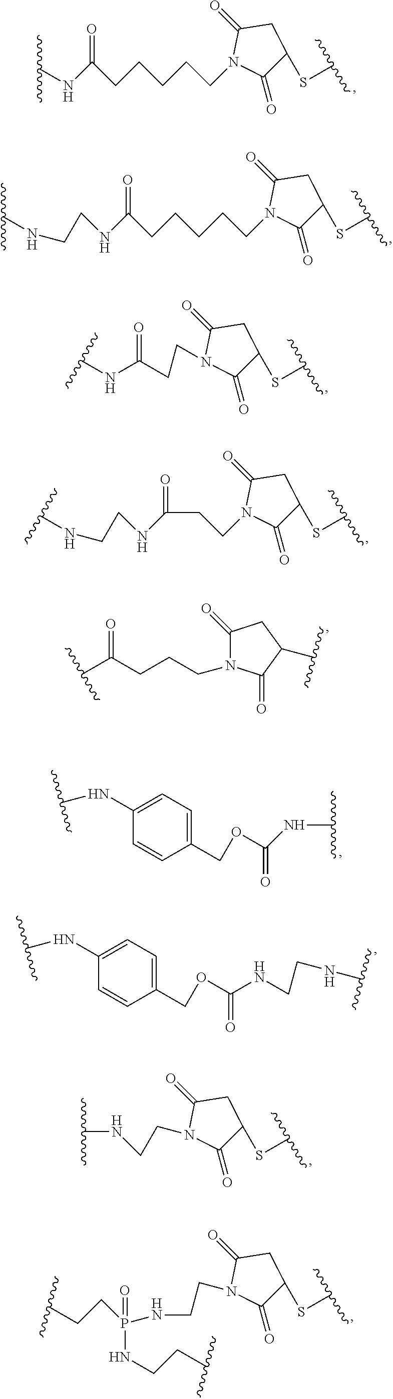 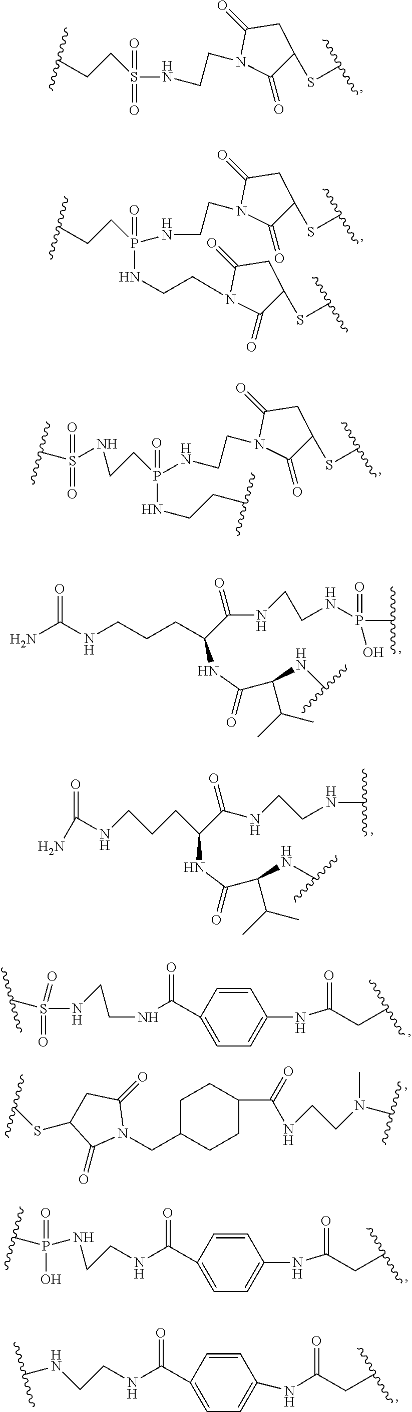 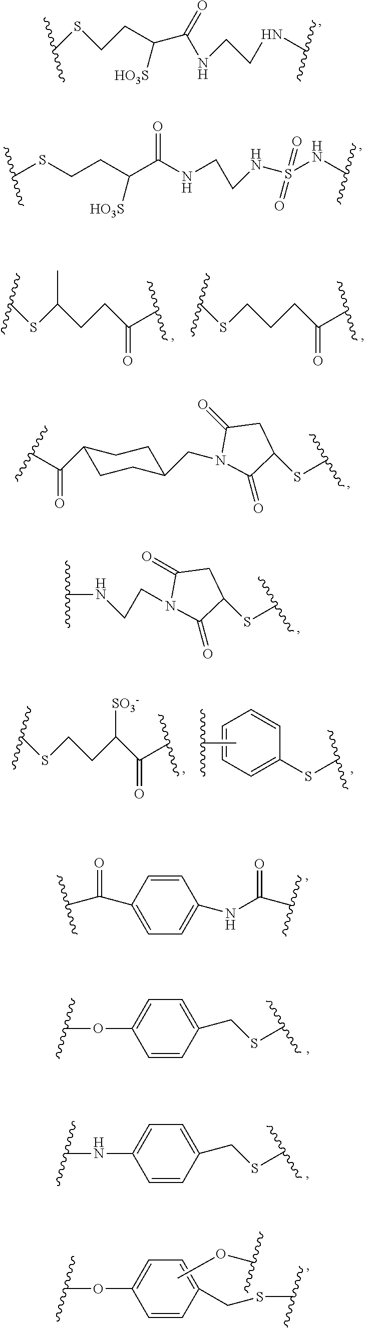 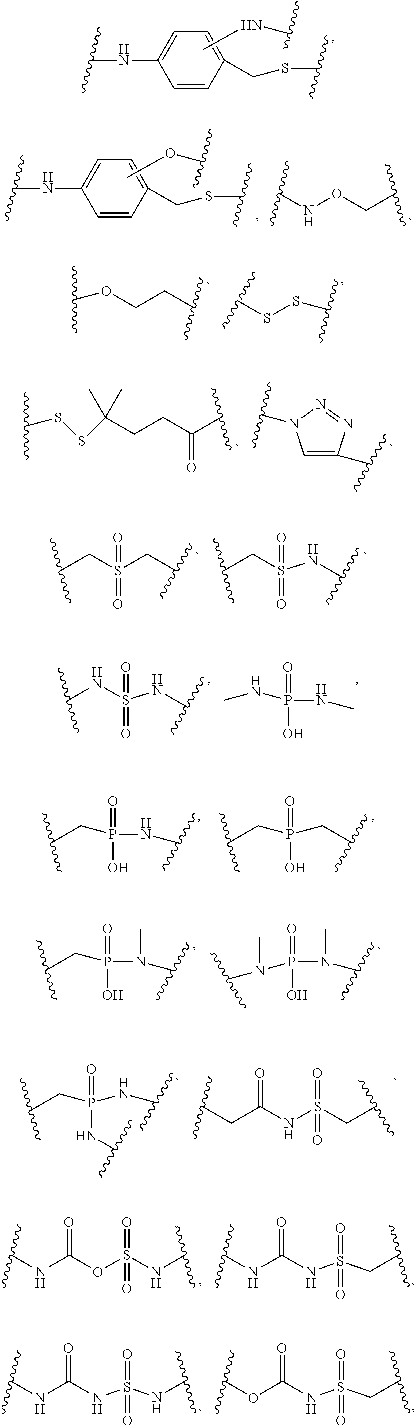 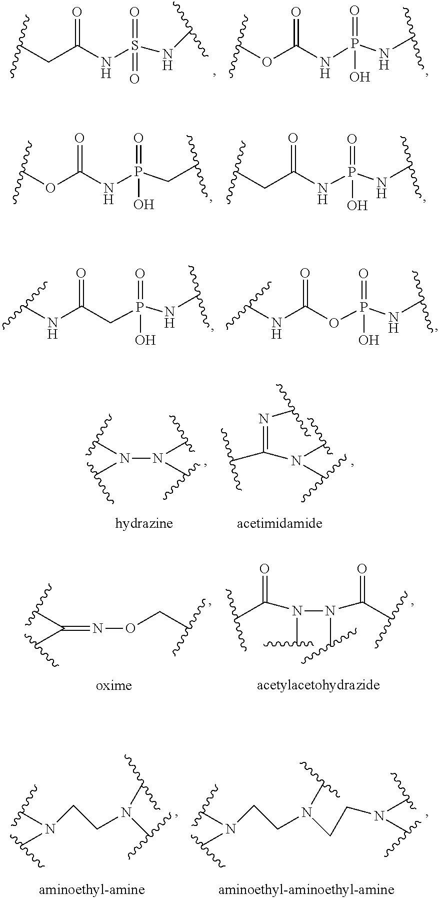 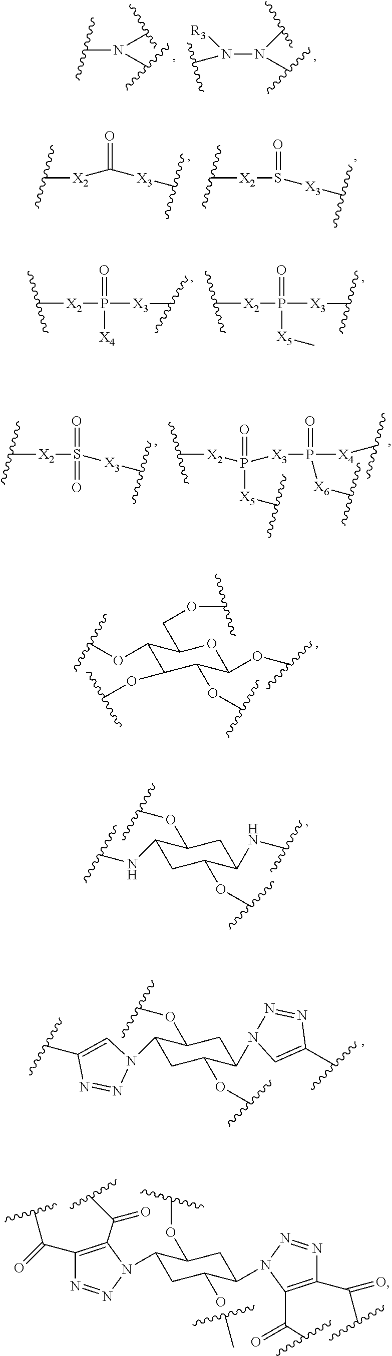 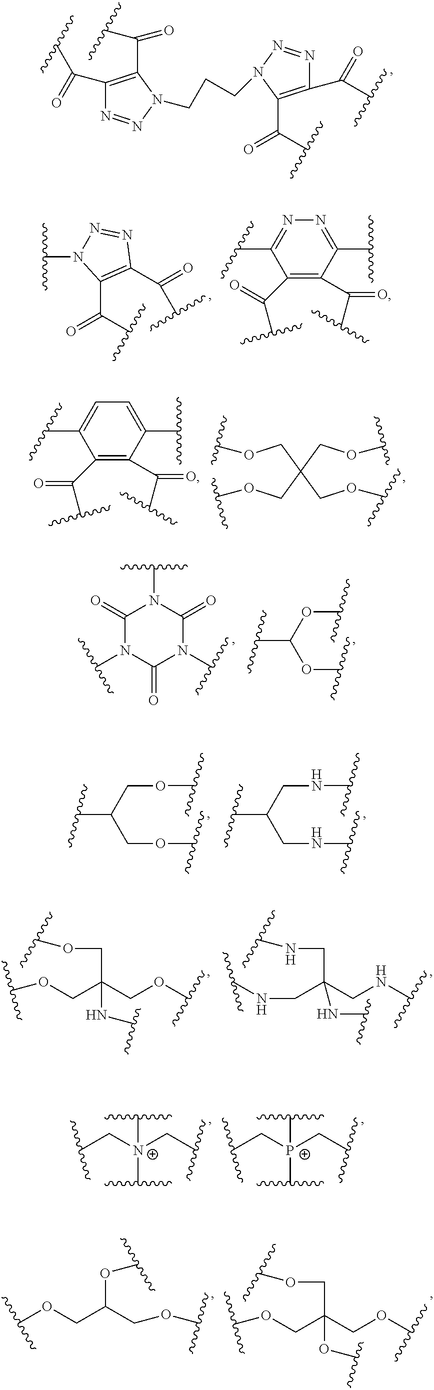 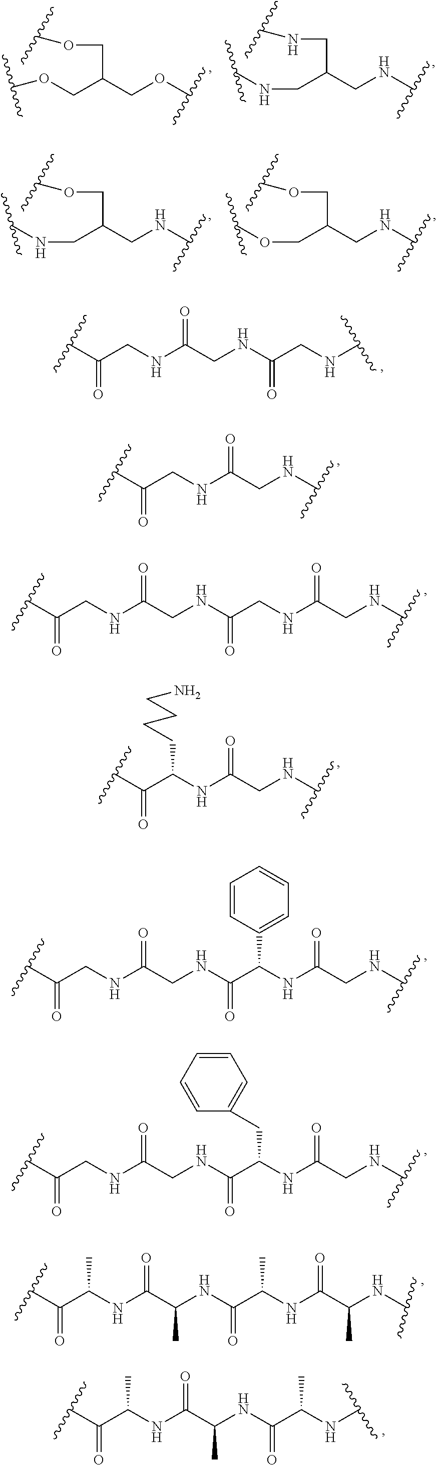 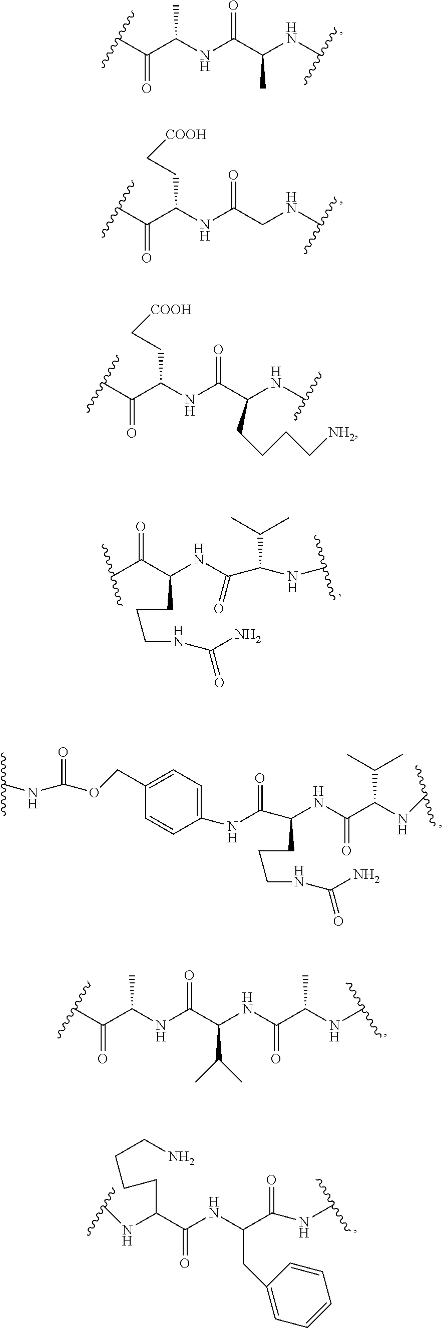 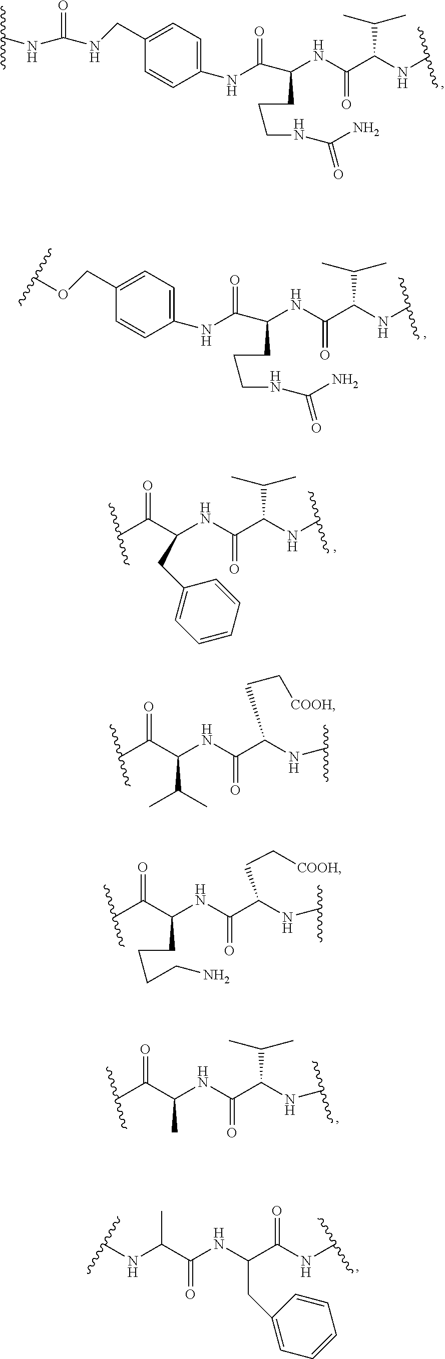
or a combination above thereof; wherein [symbol] is the site of linkage; X₂, X₃, X₄, X₅, or X₆, are independently selected from NH; NHNH; N(R₁₂); N(R₁₂)N(R₁₂—); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR¹², or 1~8 amino acids; wherein R₁₂ and R₁₂, are independently H;C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 100.

6. The antibody drug conjugate according to claim 1, wherein the structure of 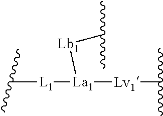
in formula (I) has a structure of formula (La); wherein the structure of 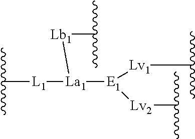
in formula (II) has a structure of formula (Ib) or (Ic); wherein the structure of 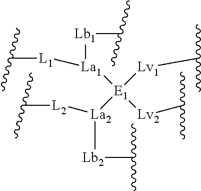
in formula (III) has a structure of formula (Id), (Ie), (If) or (Ig), or 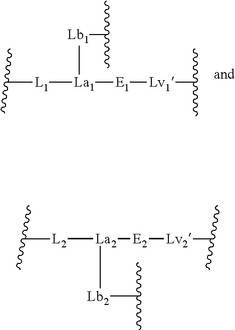
in formula (IV) has a structure of formula (Ia), illustrated as following: 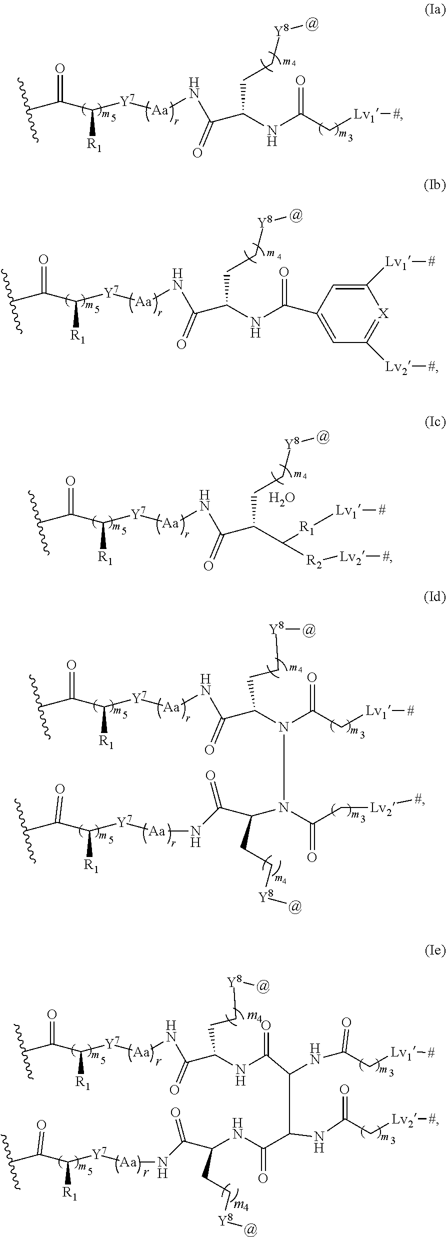 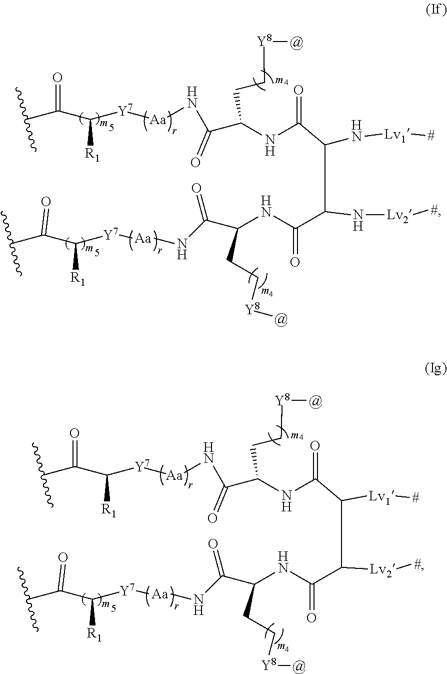
wherein “ [symbol] ” is a site that links a drug; “#” is a site that links a S (thiol), O (phenol), NH (amino), CHO (aldehyde), C(═O) (ketone), C(O)(NH) (amide) and C(O)(OH) (carboxylate) of an antibody; Aa is L- or D-natural or unnatural amino acids; “@” is a site that links Lc₁ or Lc₂ described in the formula (I), (II) and (III).
R₁ is H, C₁-C₈alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(=NH)NH₂;
r is 0-12; when r is not 0, (Aa)ᵣ is the same or different amino acids or peptide units;
m₁=1-18; m₂=1-100; m₃=1-8; m₄=0-8; m₅=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y⁸ is NHC(═O), NHS(O₂), NH(SO), NHS(O₂)NH, NHP(O)(OH)NH, C(O)NH, OC(O)NH, NHC(O)NH, C(O), N, NH, CH₂, or CH;
Lv₁′ and Lv₂′ are independently selected from: 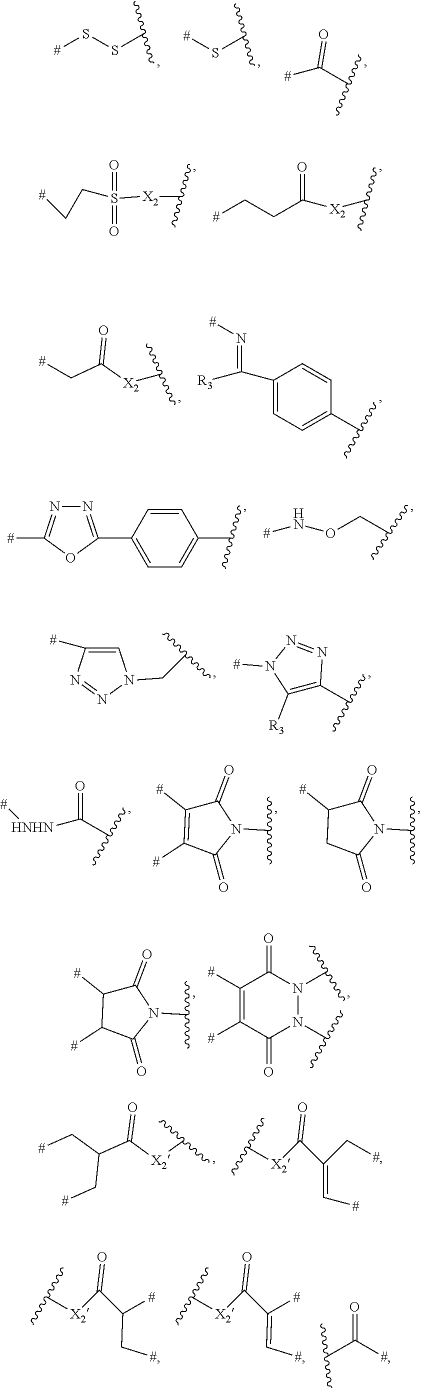 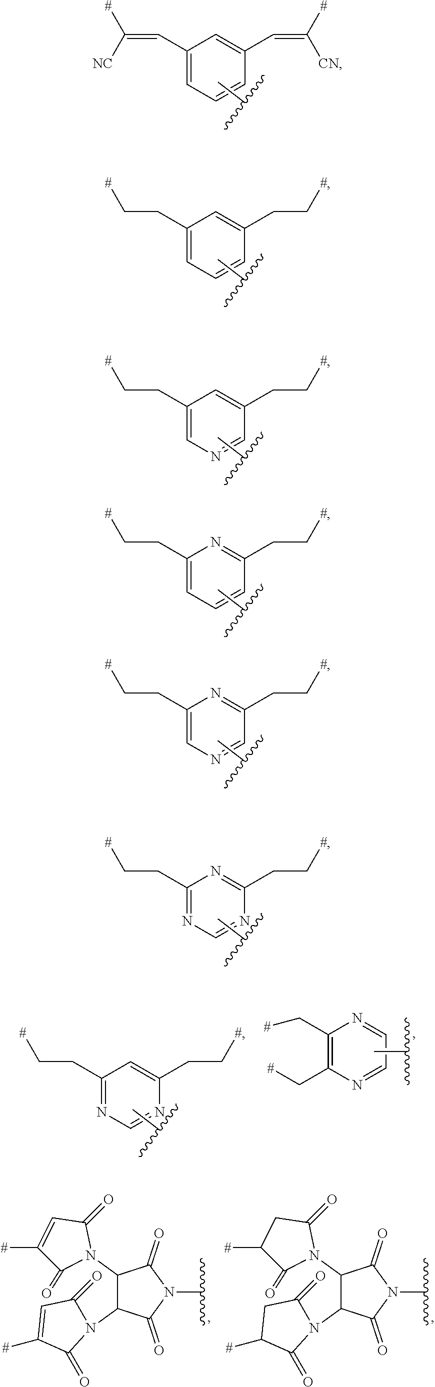 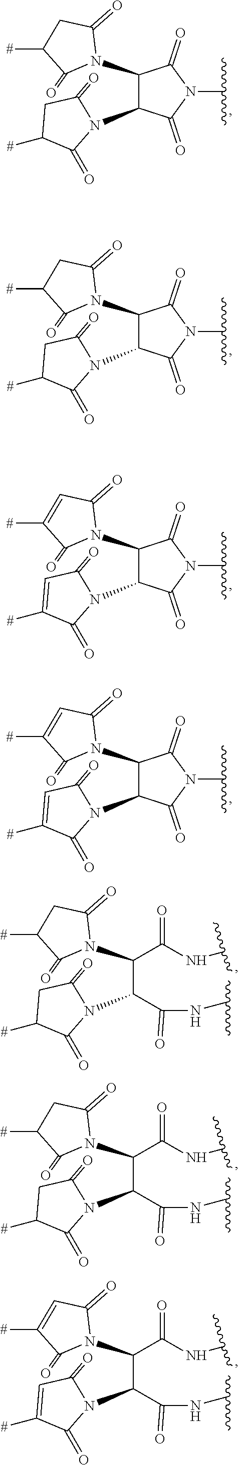 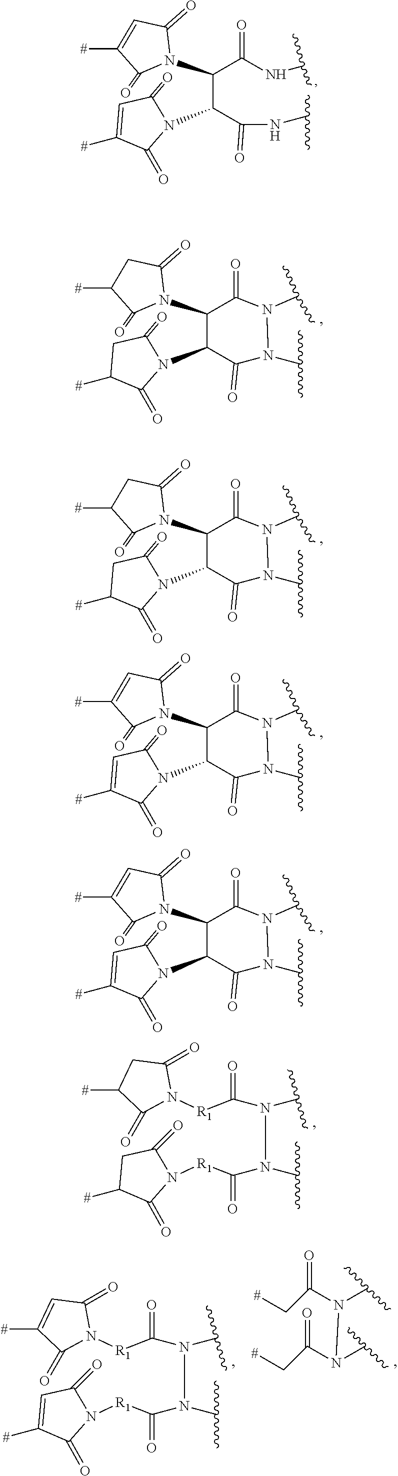 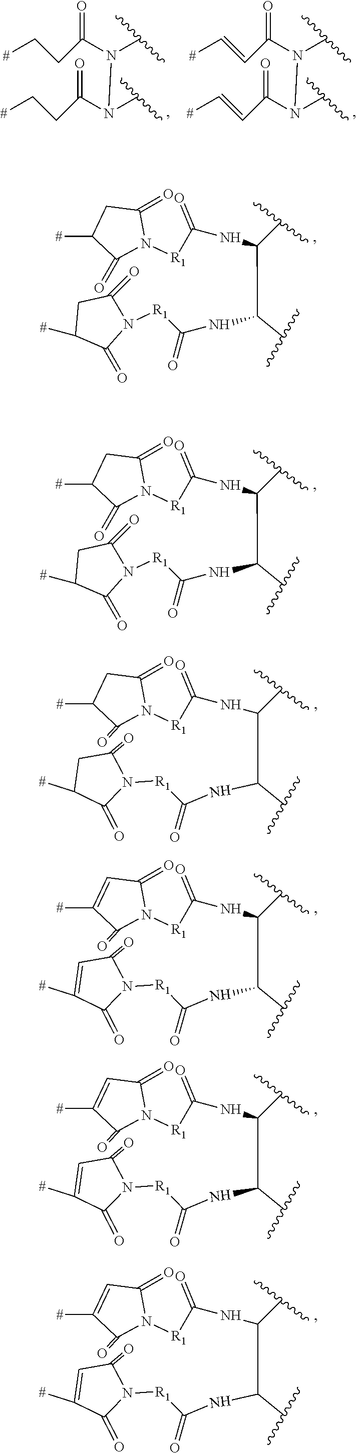 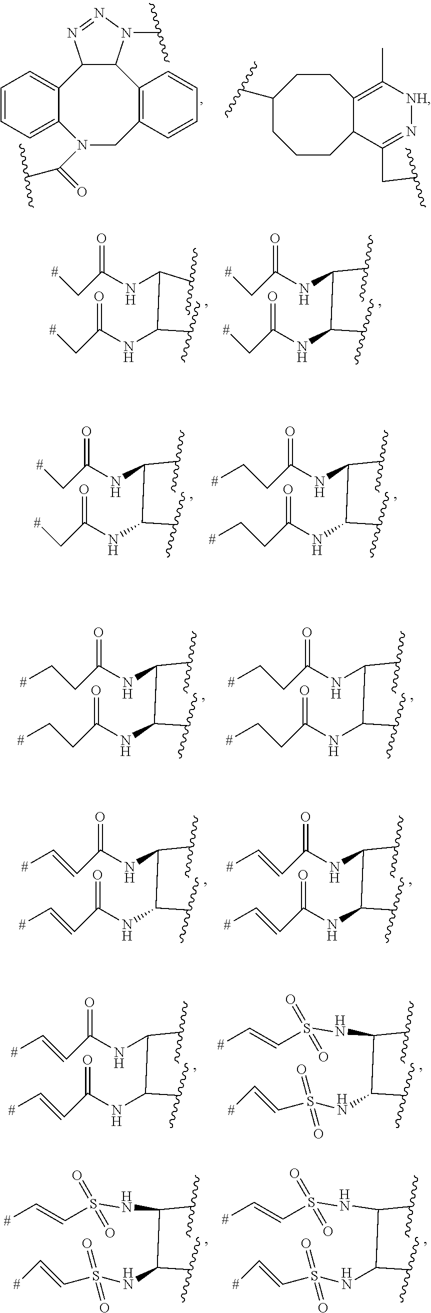 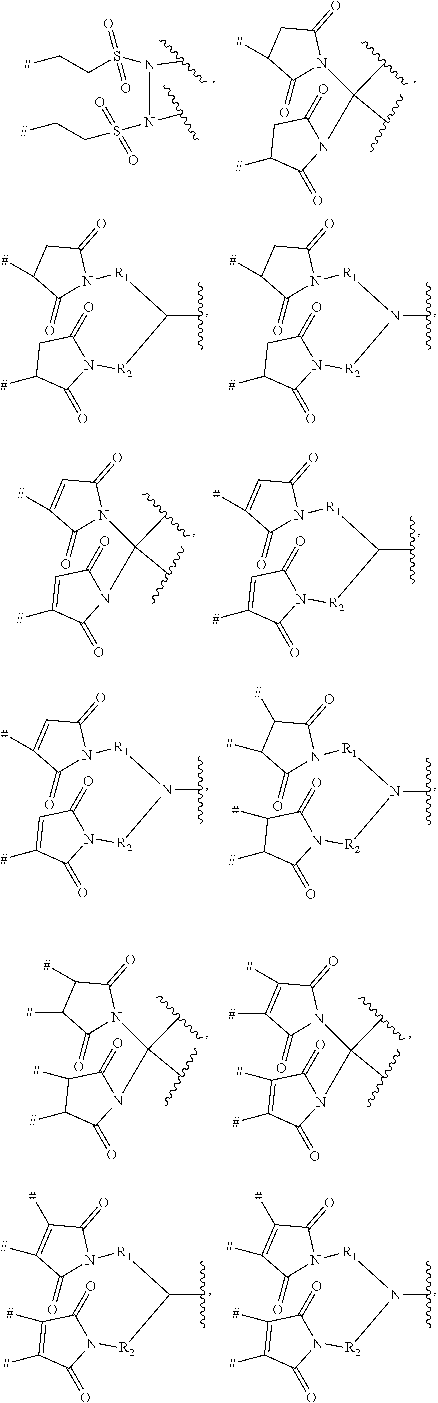 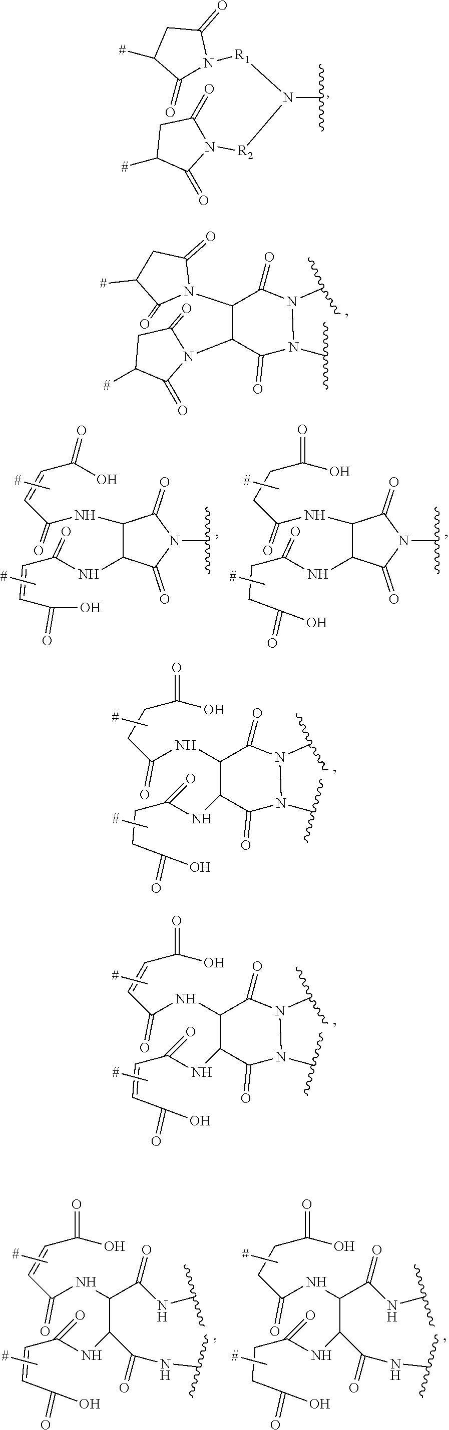 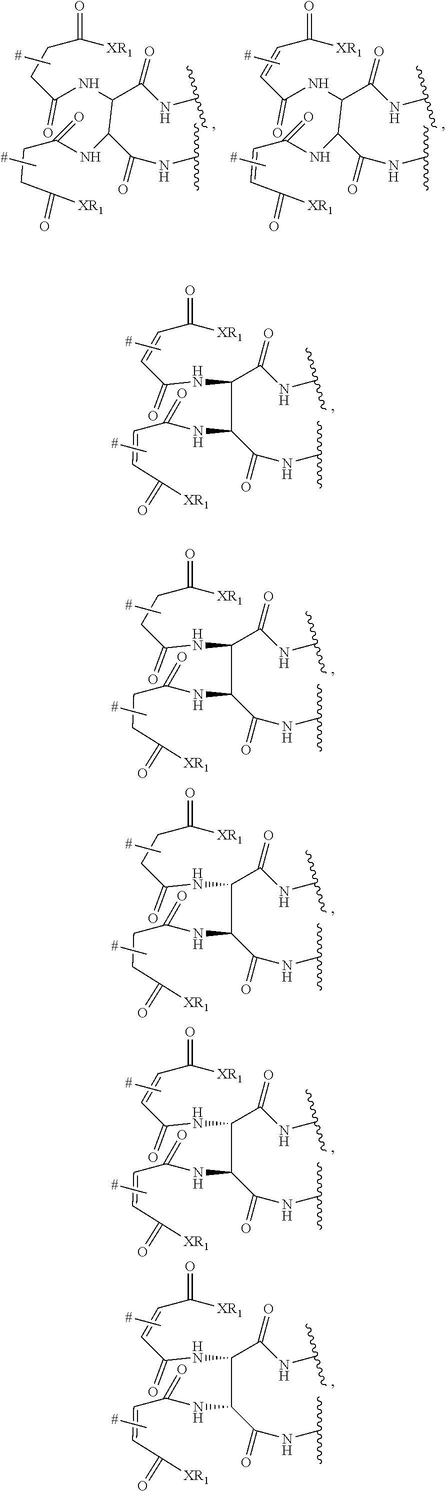 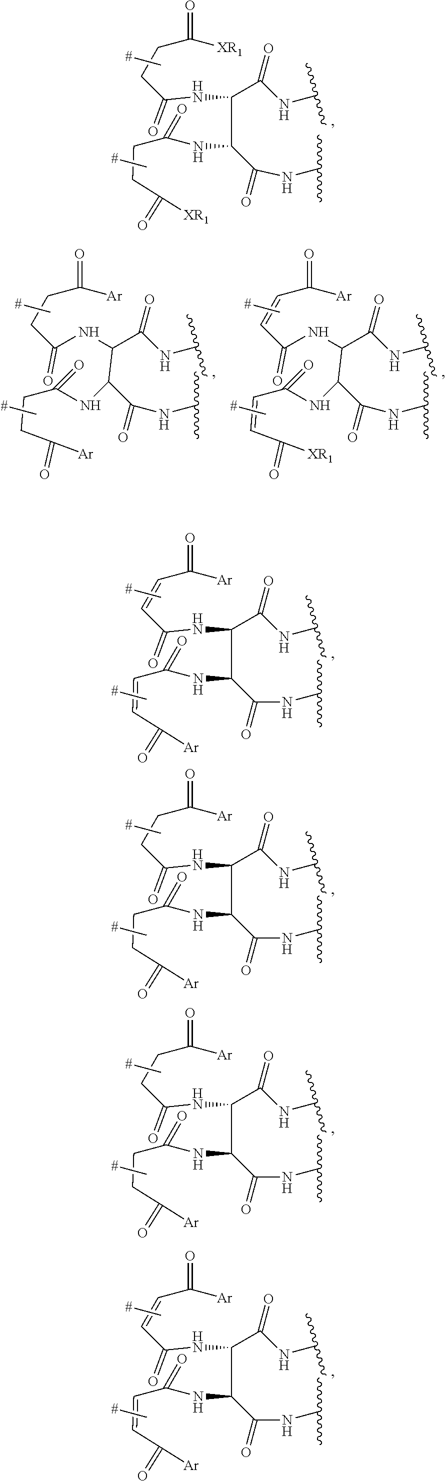 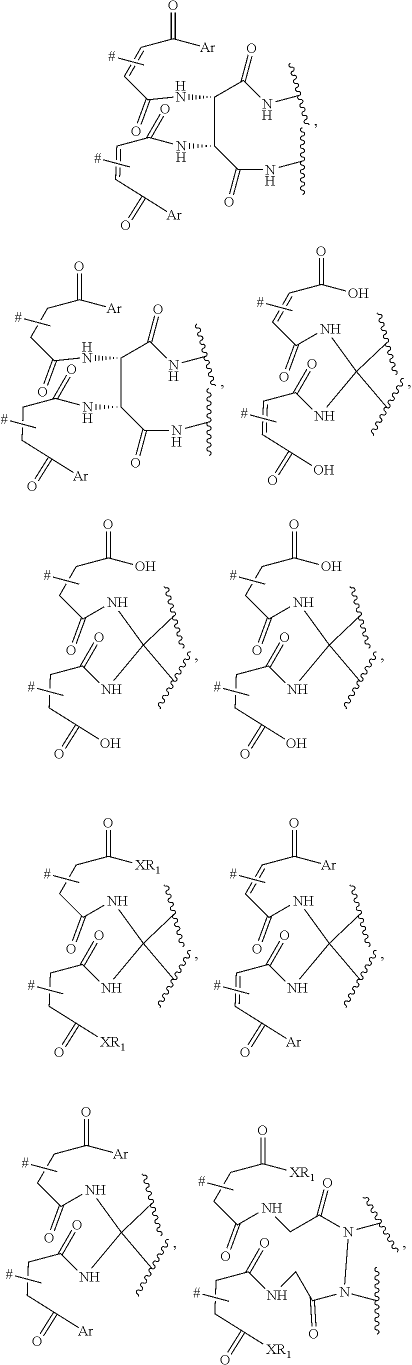 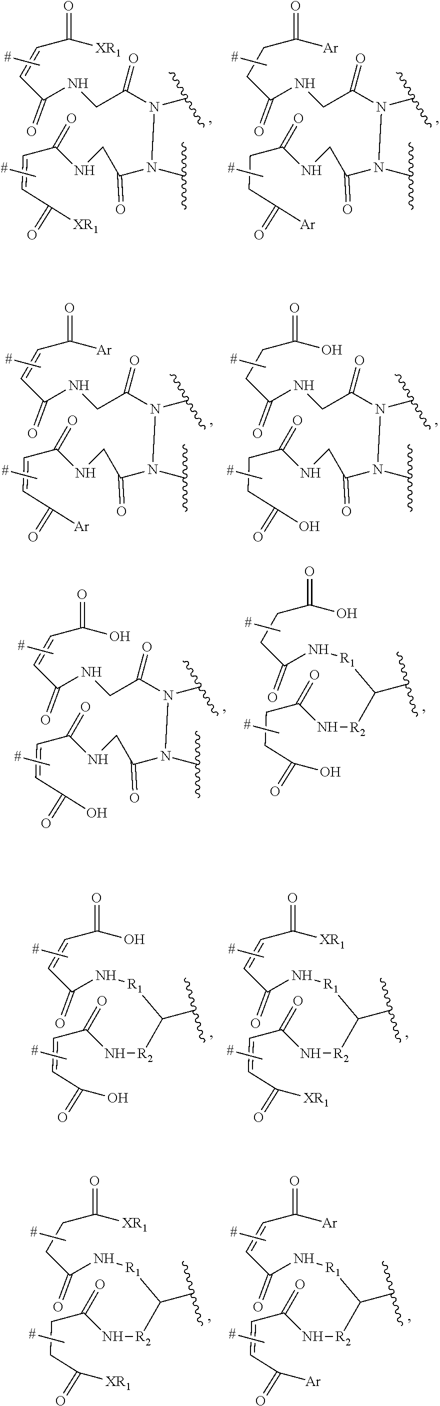 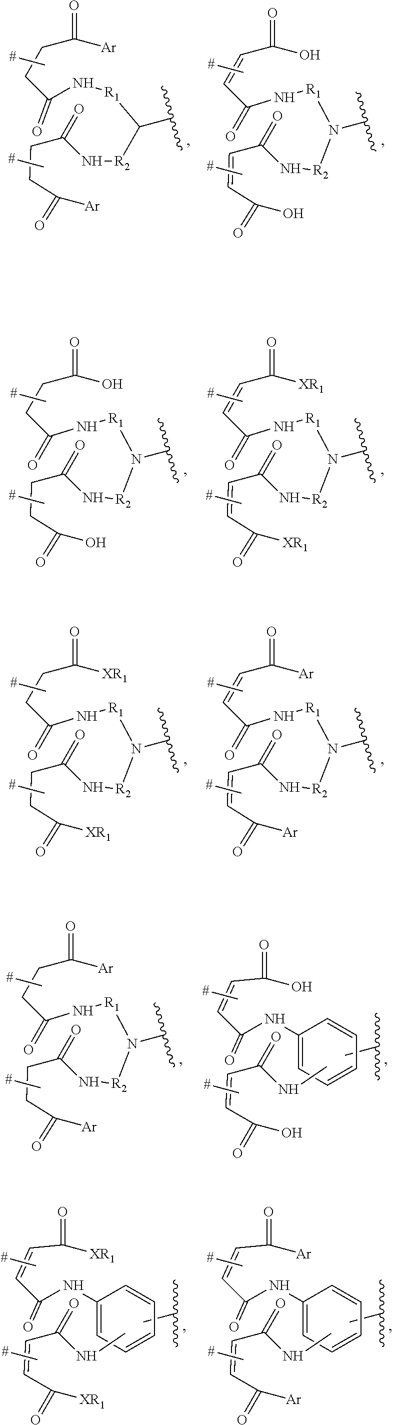 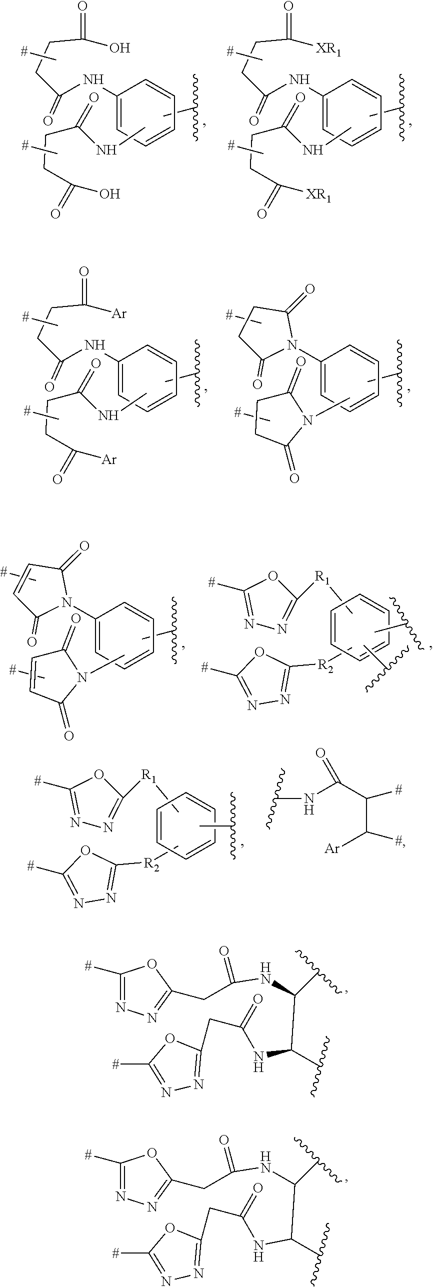 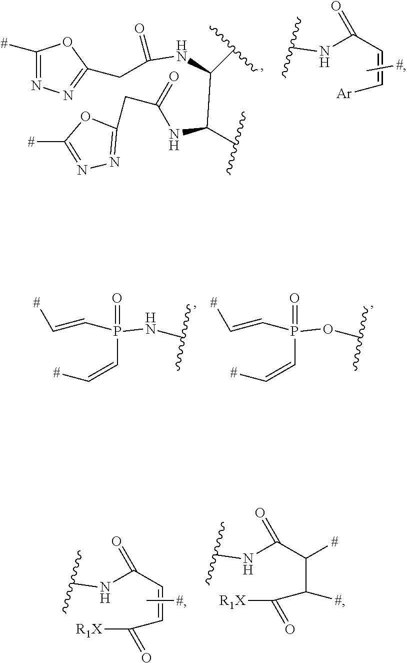
wherein “ [symbol] ” a site that links to the linker component; “#” is the site as Lv₁′ and Lv₂′ indicated in the formulas that links a S (thiol), O (phenol), NH (amino), CHO (aldehyde), C(═O) (ketone), C(O)(NH) (amide) and C(O)(OH) (carboxylate) of an antibody; wherein R₁, X₁′ and X₂′are described above; X is 0, NH, S, CH₂; the conneting bond “—” in the middle of the two atoms means it can link either one of the two atoms, Ar is an aromatic group.

7. The antibody drug conjugate according to claim 1, the core linker structure (L₁″) having an affinity ligand in formula (I): 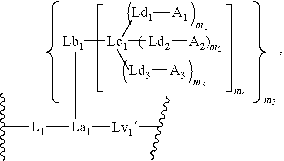
wherein (L₁″), is further selected from Formula (Ia′): 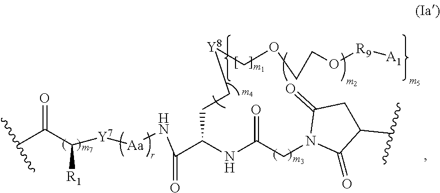
wherein Aa is L- or D-natural or unnatural amino acids; A₁ is the affinity ligand defined the same in claim 1;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H4OH, CH(OH)CH₃, CH₂C(O)NH₂, CH₂CH₂C(O)NH₂, CH₂CH₂CH₂NHC(═NH)NH₂;
r is 0-12; when r is not 0, (Aa)ᵣ is the same or different amino acids or peptide units;
m₁=1-18; m₂=1-100; m₃=1-8; m₄=0-8; m₅=1-6; m₇=1-8;
Y⁷ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y⁸ is NHC(═O), NH, O, NHS(O₂), NH(SO), NHS(O₂)NH, NHP(O)(OH)NH, 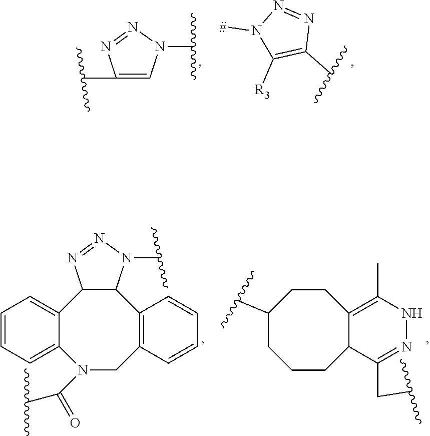
C(O)O, C(O), OC(O)NH, C(O)NH, or Ar;
R₉ is (O═)CR₁, (O═)CNHR¹, NHC(═O), NH, O, NHS(O₂), NH(SO), NHS(O₂)NH, NHP(O)(OH)NH or C(O)NH, R₁(COCH₂NH)ₘ₄H, R₁(Aa)ᵣ, (Aa)ᵣ, C(O), Ar or 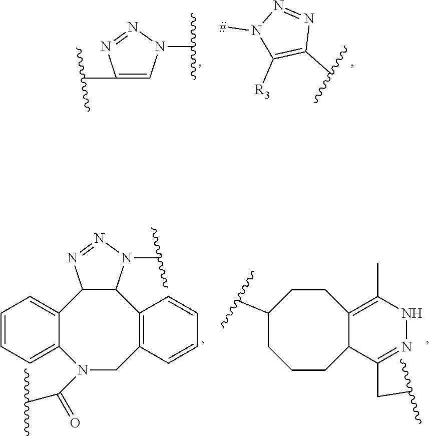
wherein R₃ is H, C₁-C₈alkyl, ester, amide, Ar, ketone, alkyl acid, alkyl alcohol, alkyl amine, CH₂C₆H₅, CH₂C₆H4OH, CH(OH)CH₃, CH₂C(O)NH₂, CH₂CH₂C(O)NH₂, CH₂CH₂CH₂NHC(═NH)NH₂; R₁ is defined above.

8. The antibody drug conjugate according to claim 1, the core linker structure (called L₁″ and L₂″ fused jointly) having an affinity ligand in Formula (III): 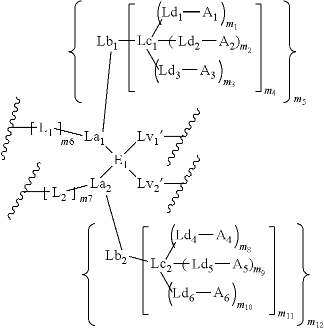
(L₁″ and L₂″ fused jointly) is further selected from the following formula (Ib) and (Ic): 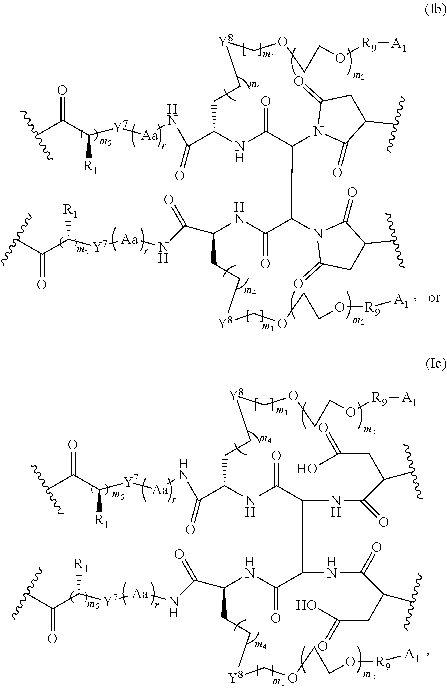
wherein R₁, Y⁷, Y⁸, R⁹, A₁, Aa, r, m₁, m₂, m₄, and m₅ are defined the same in claim 6.

9. The antibody drug conjugate according to claim 1, the antibody drug conjugate with a branch affinity ligand having the following structures: 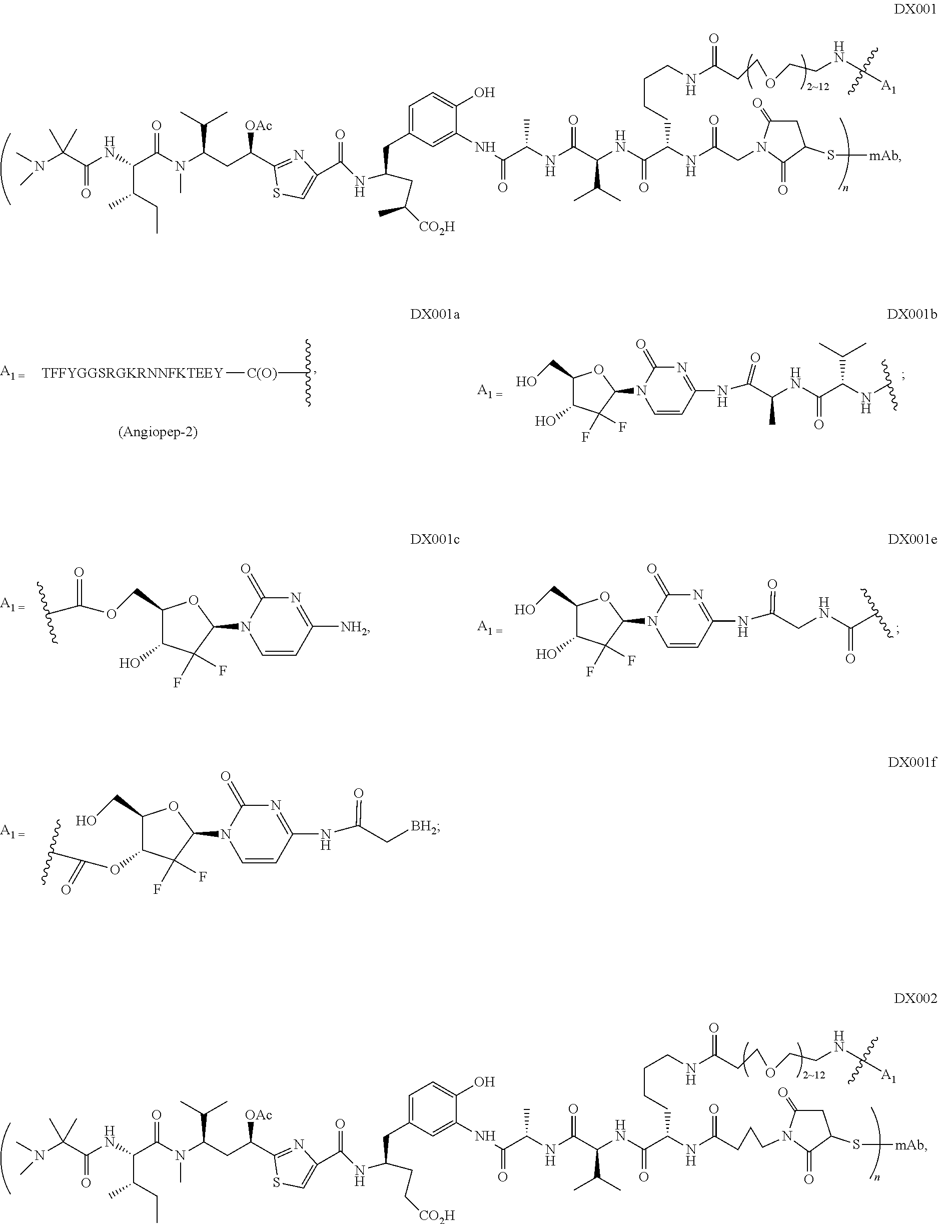 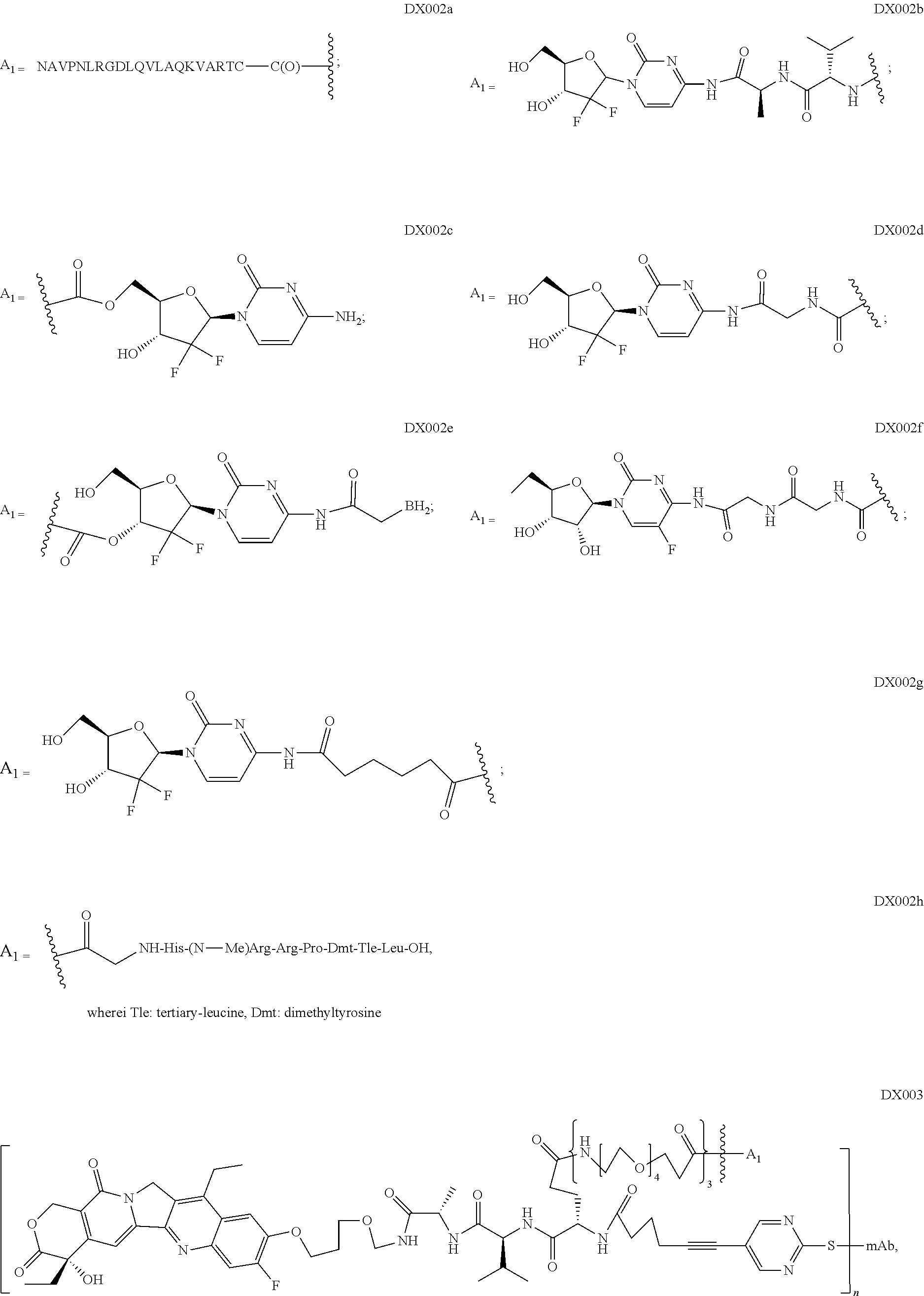 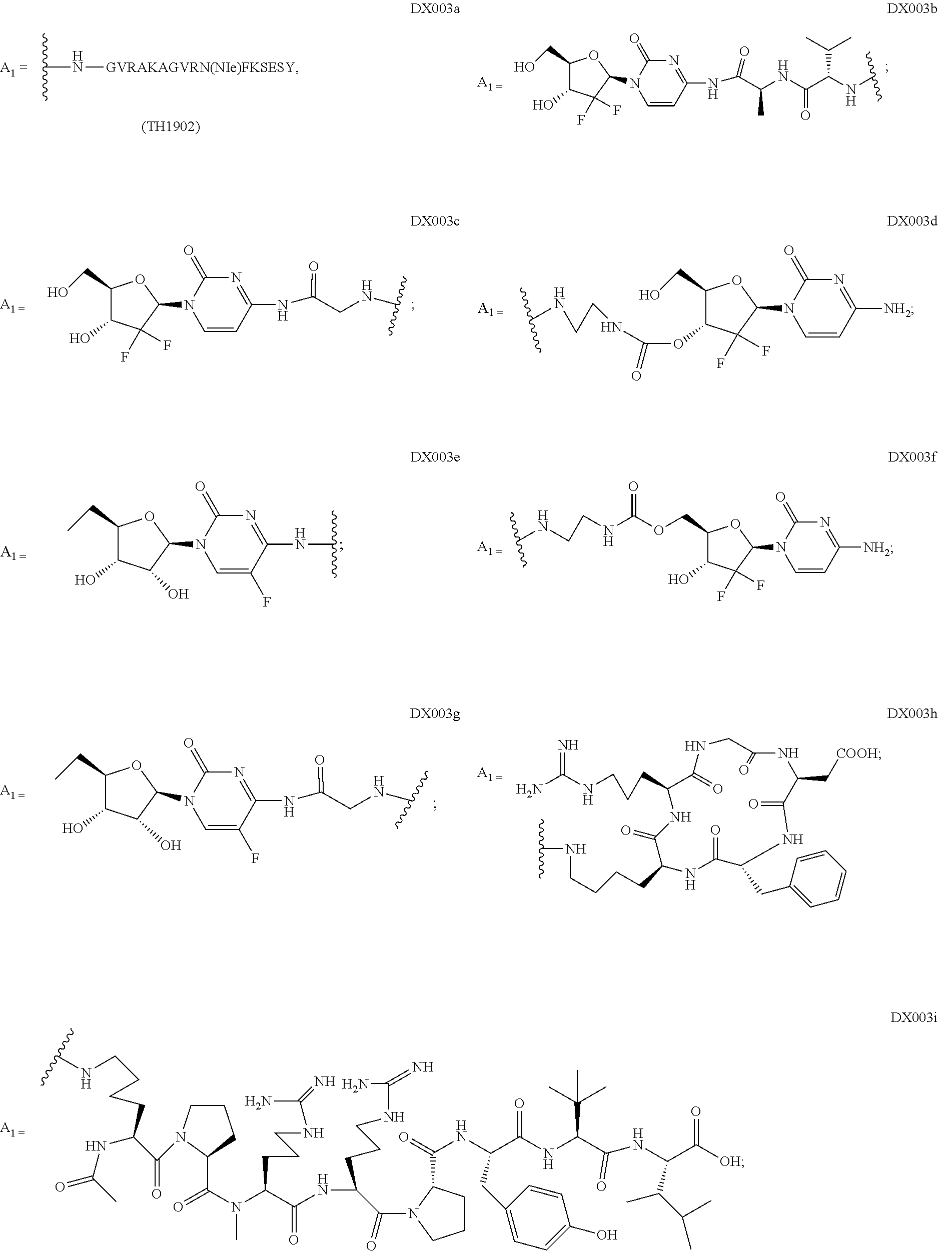 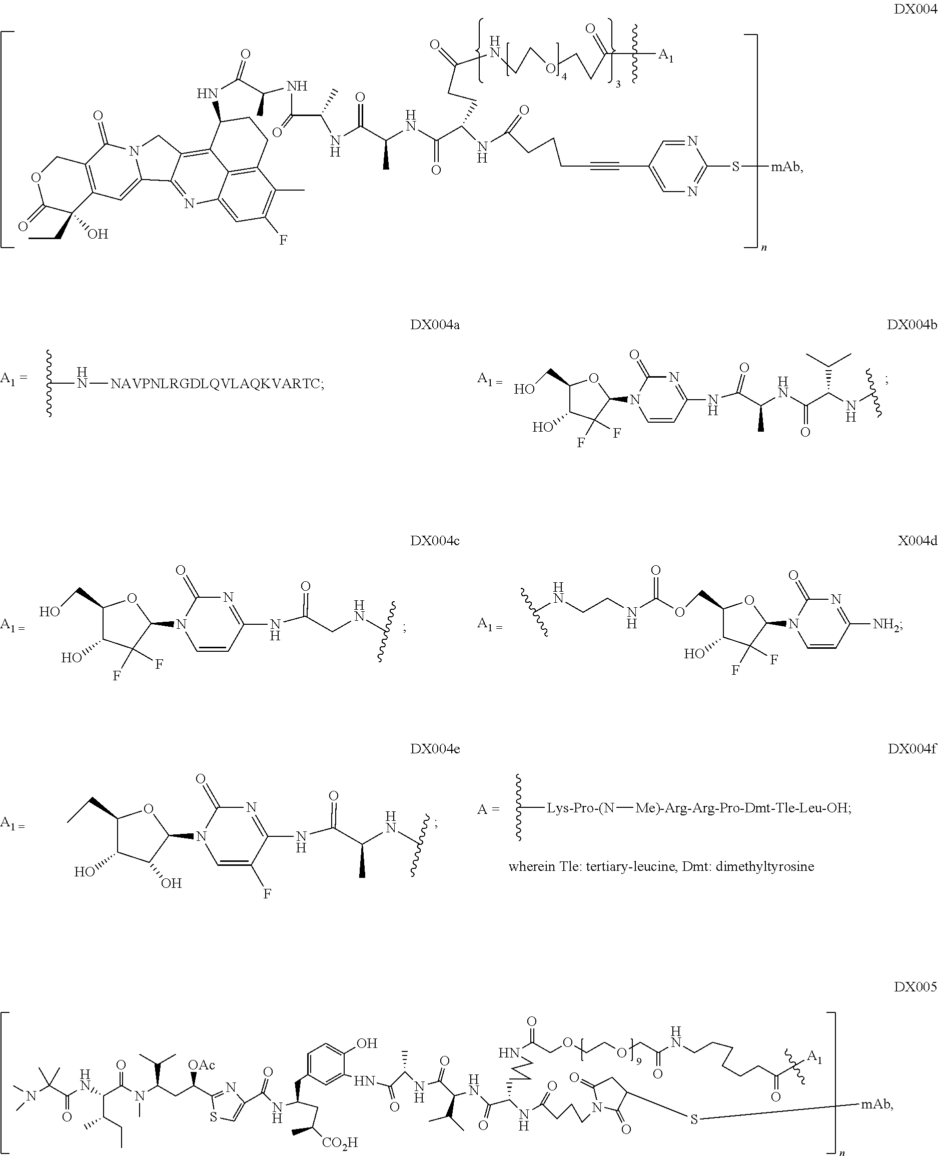 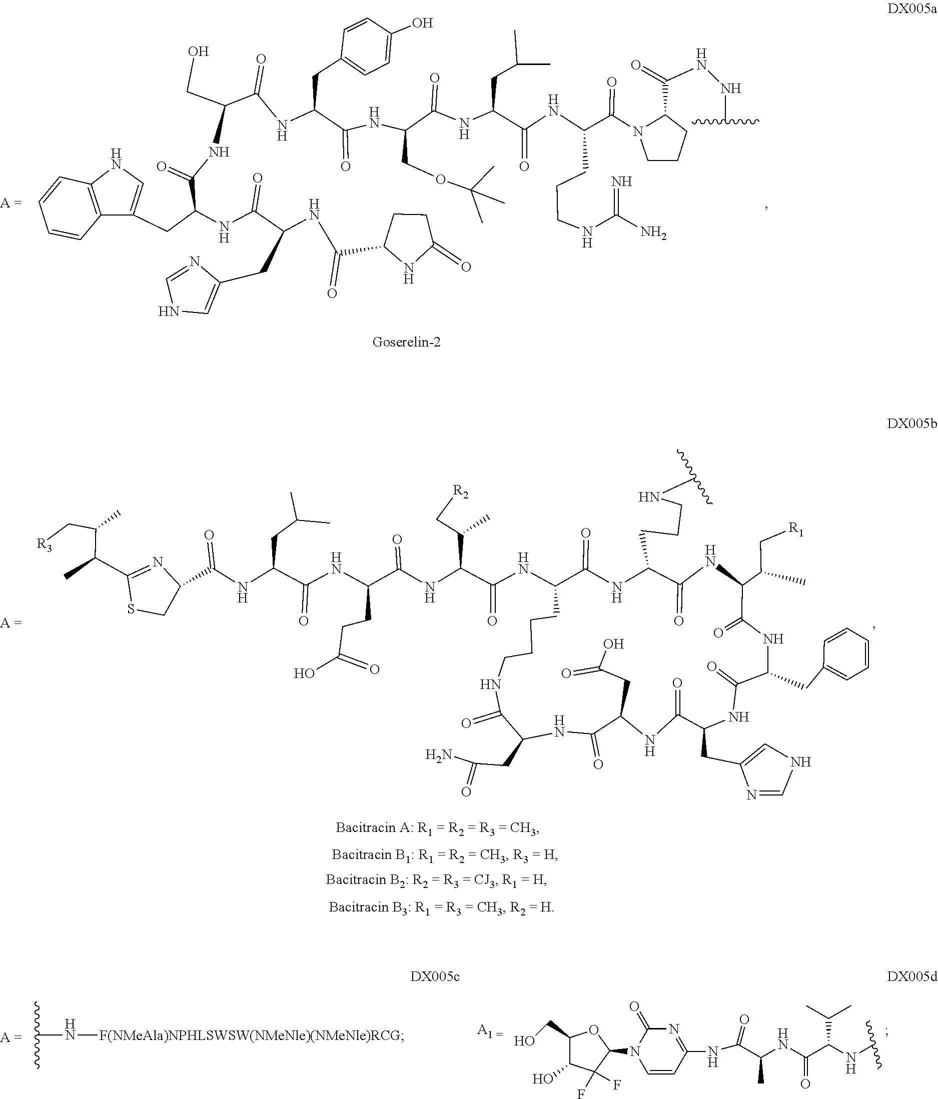 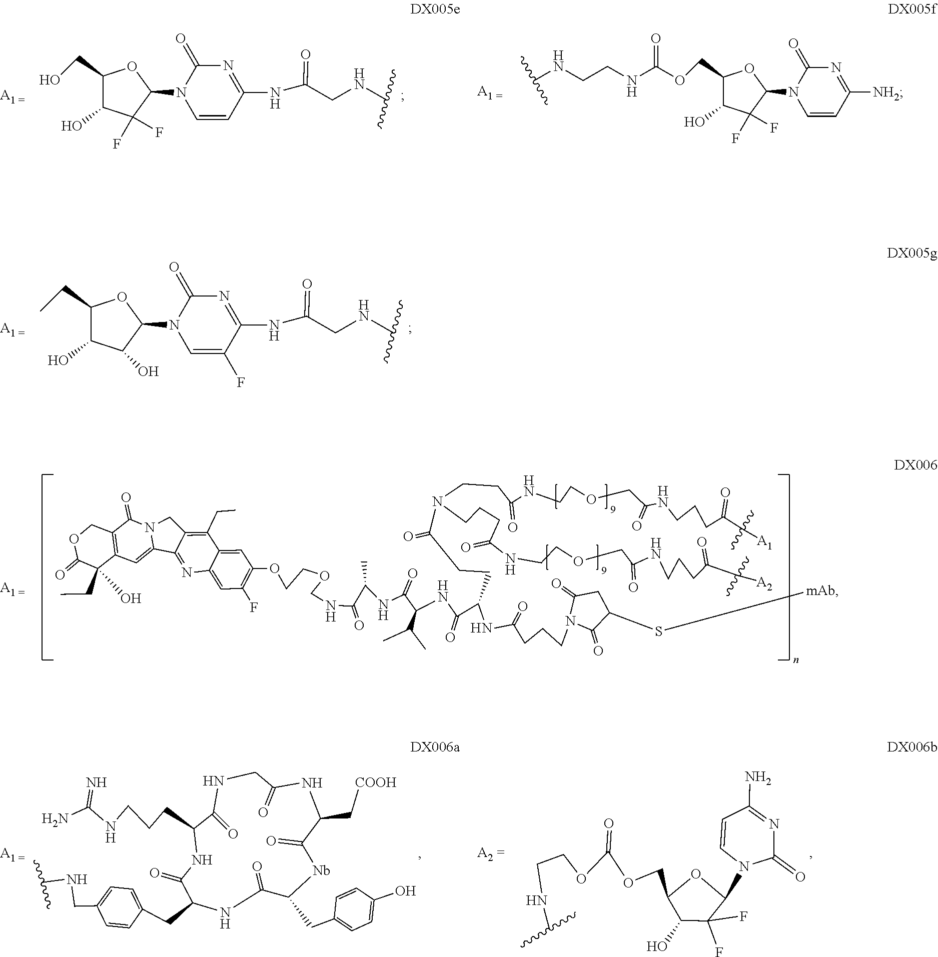 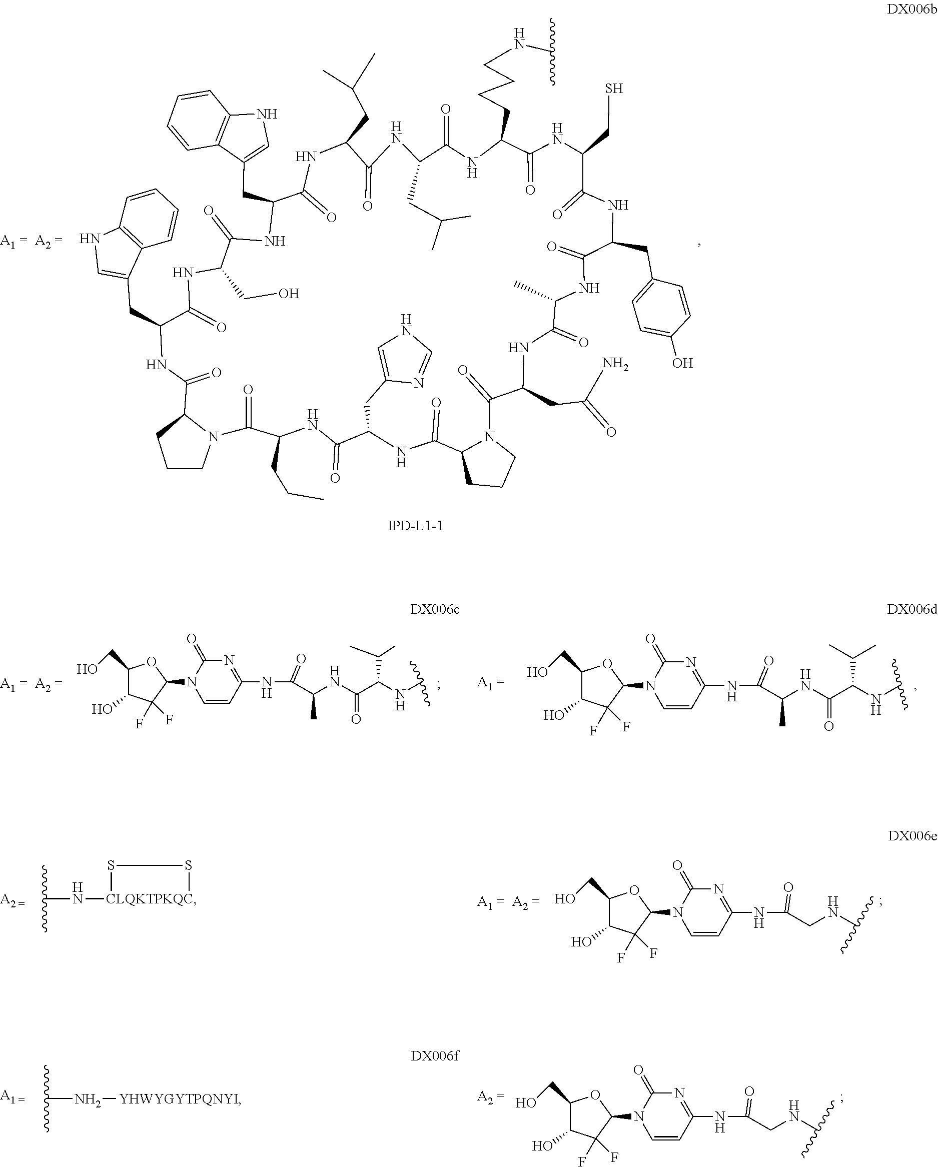 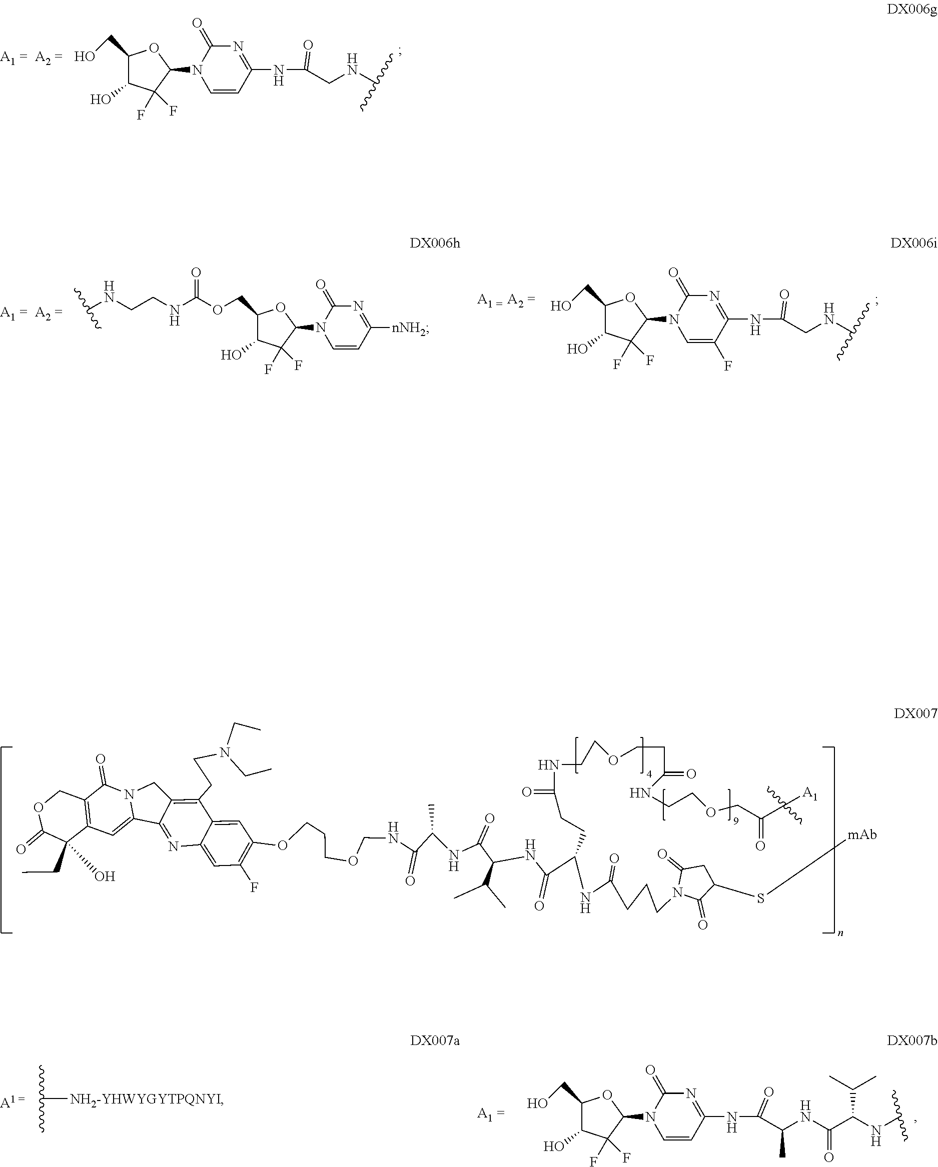 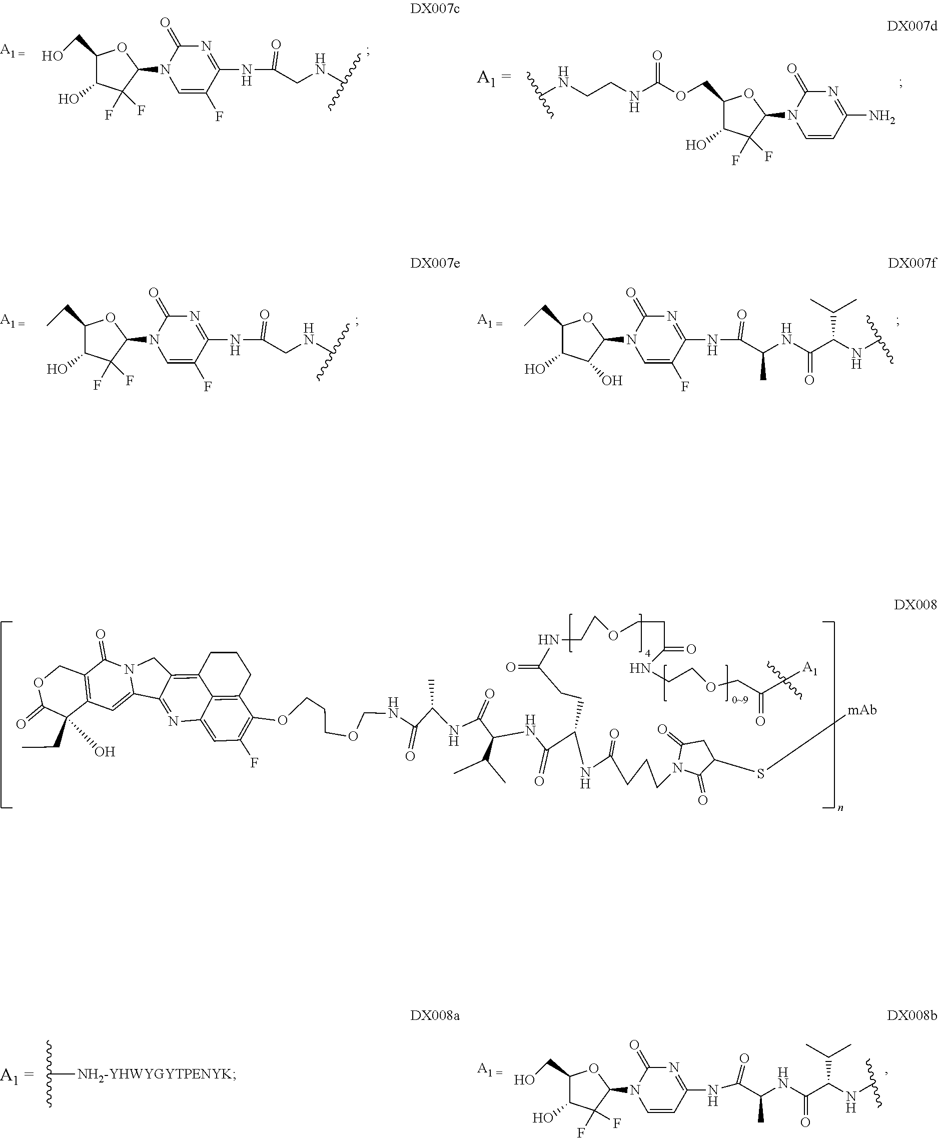 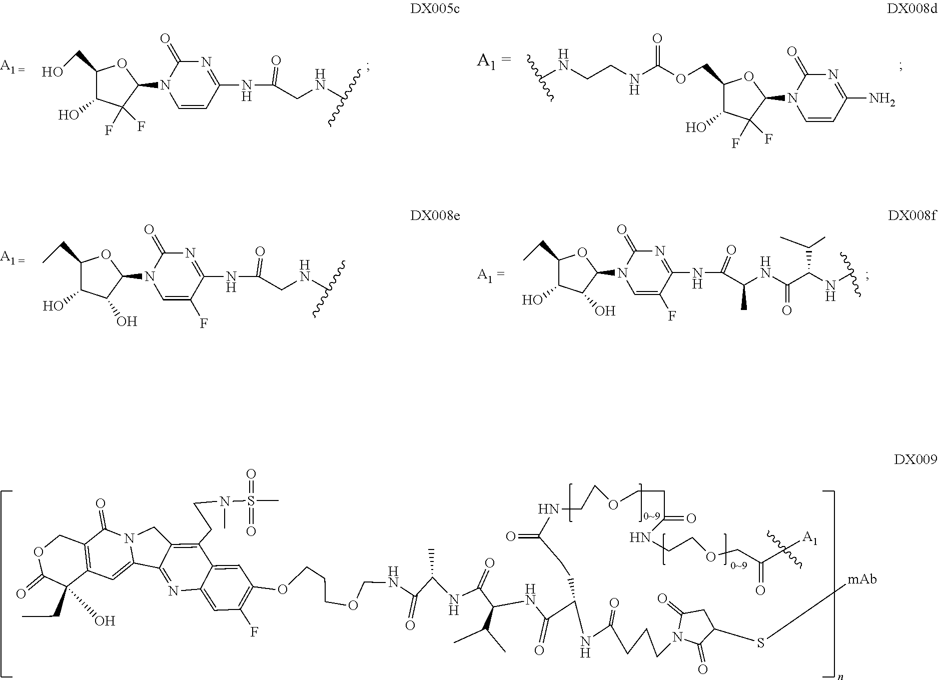 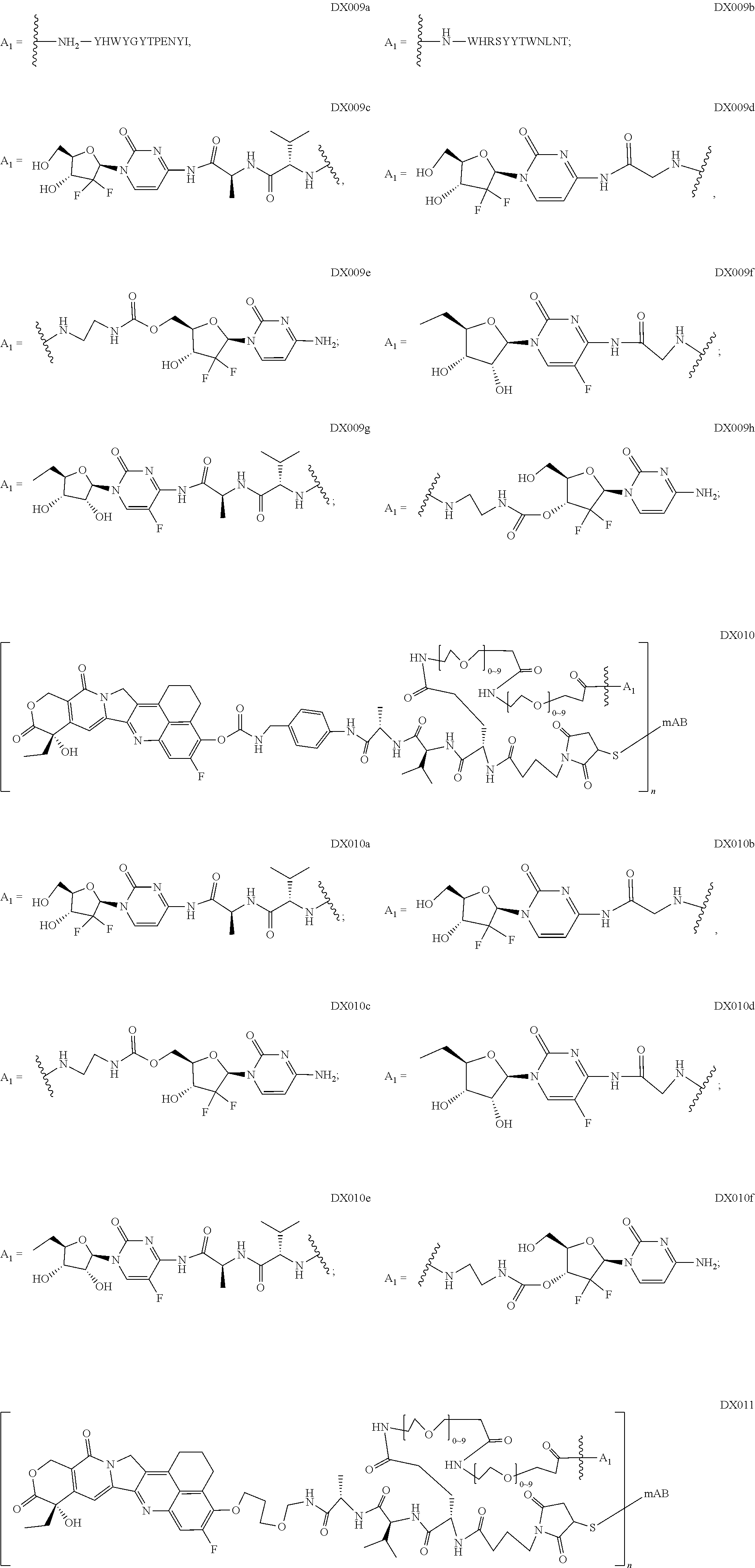 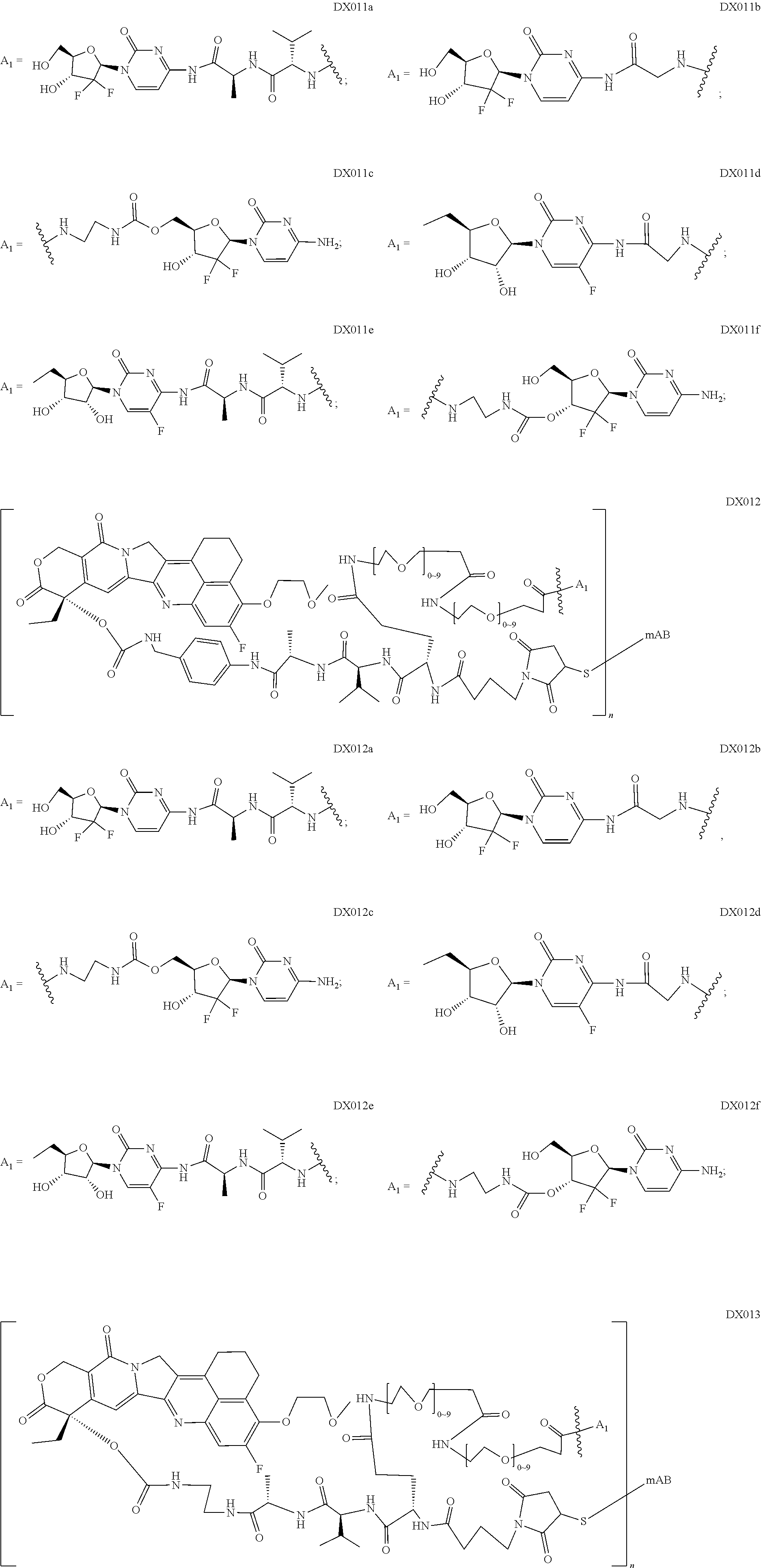 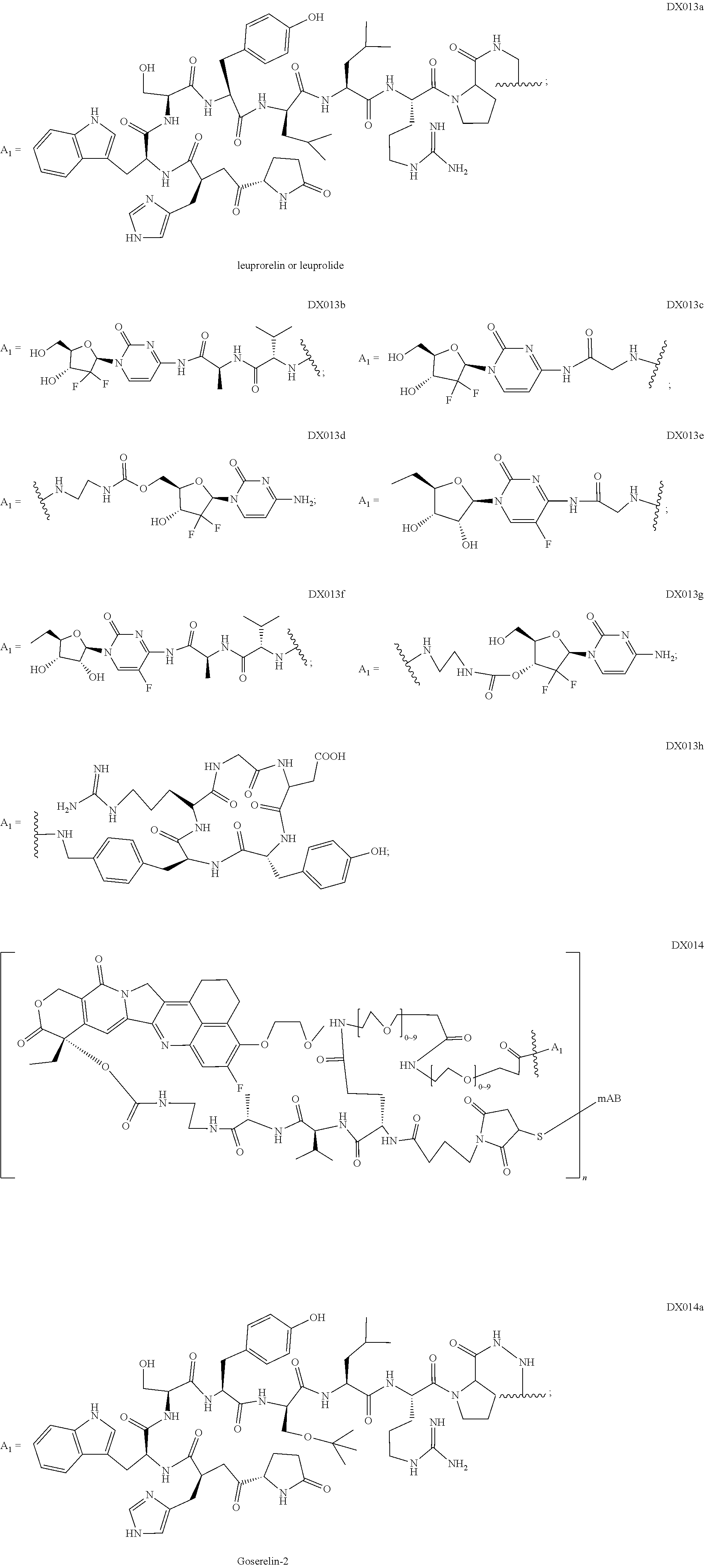 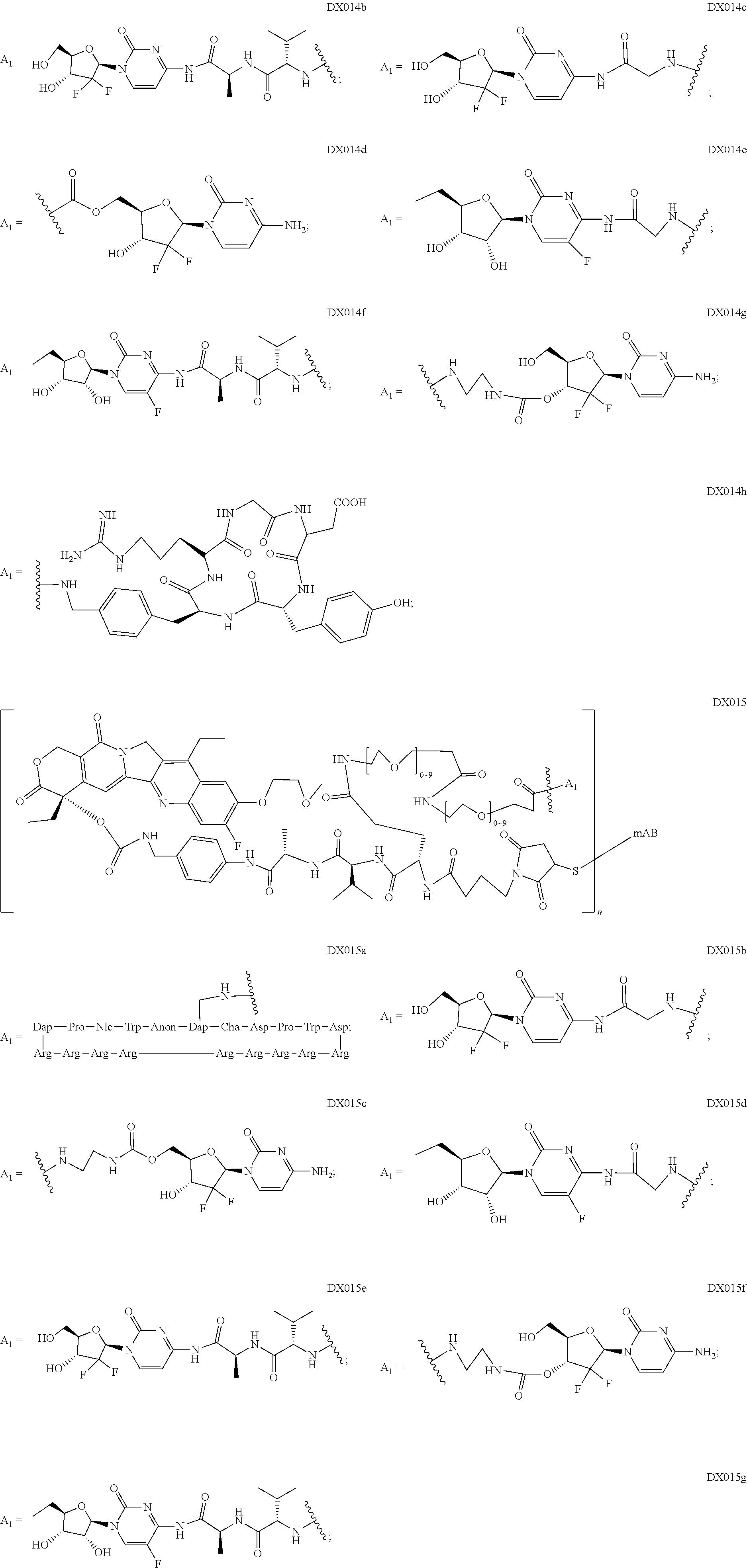 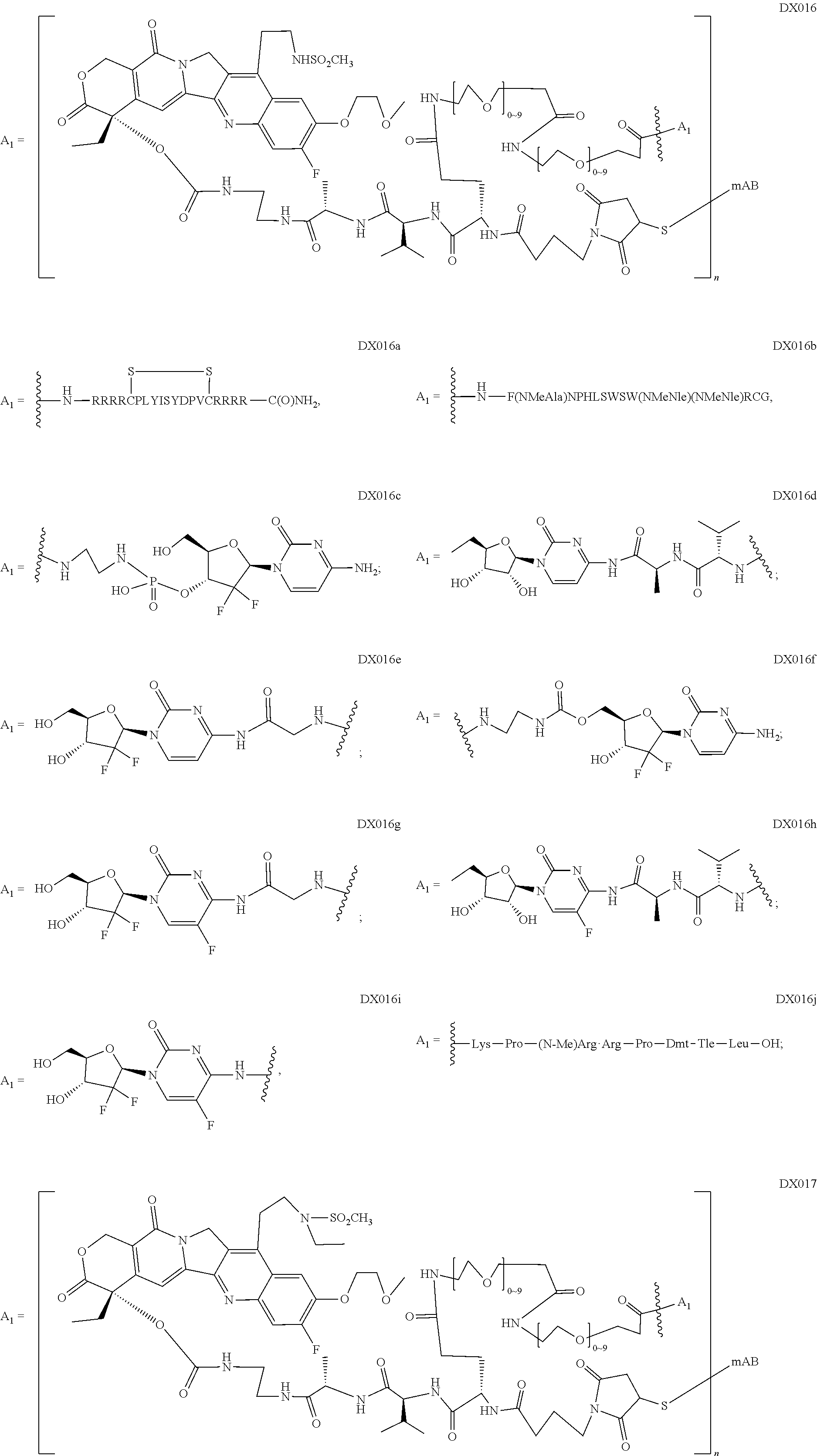 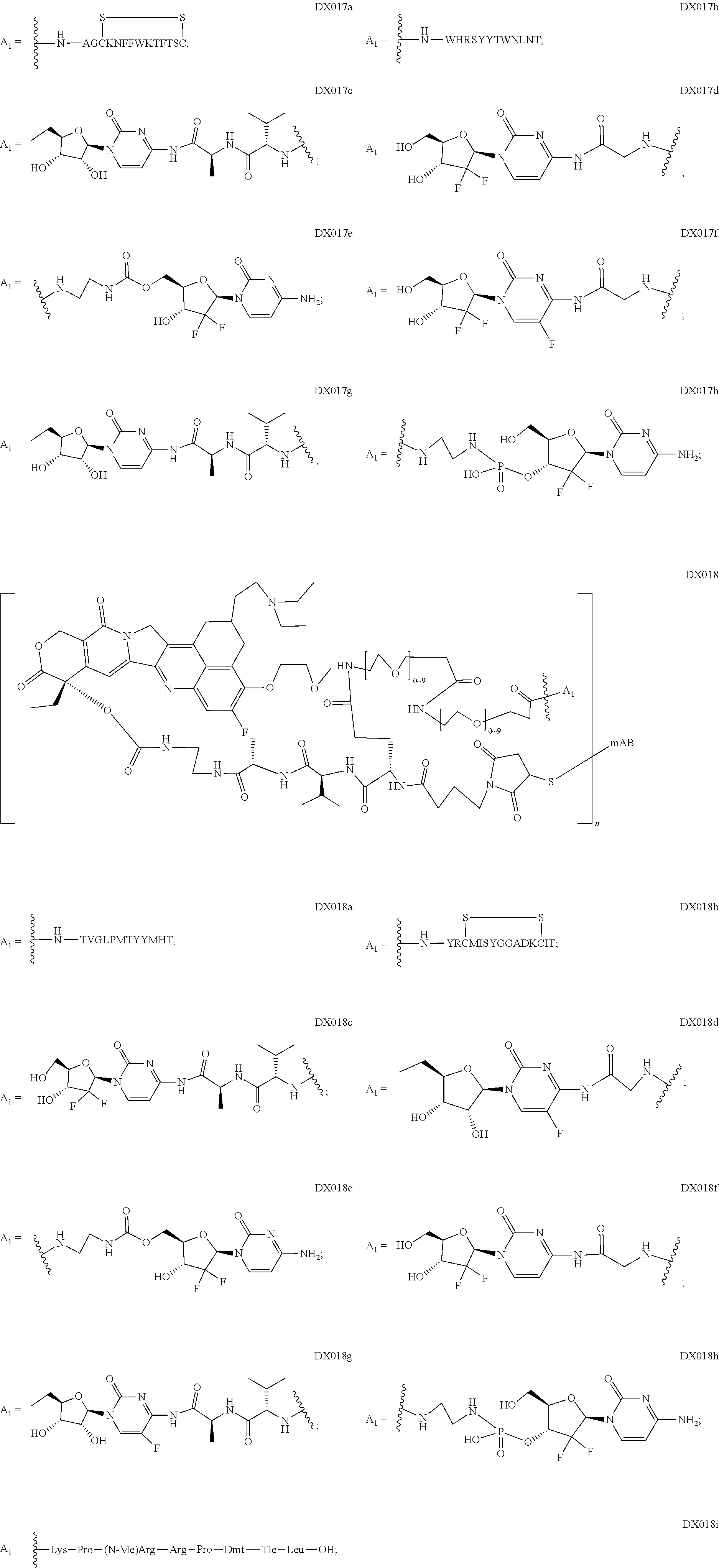 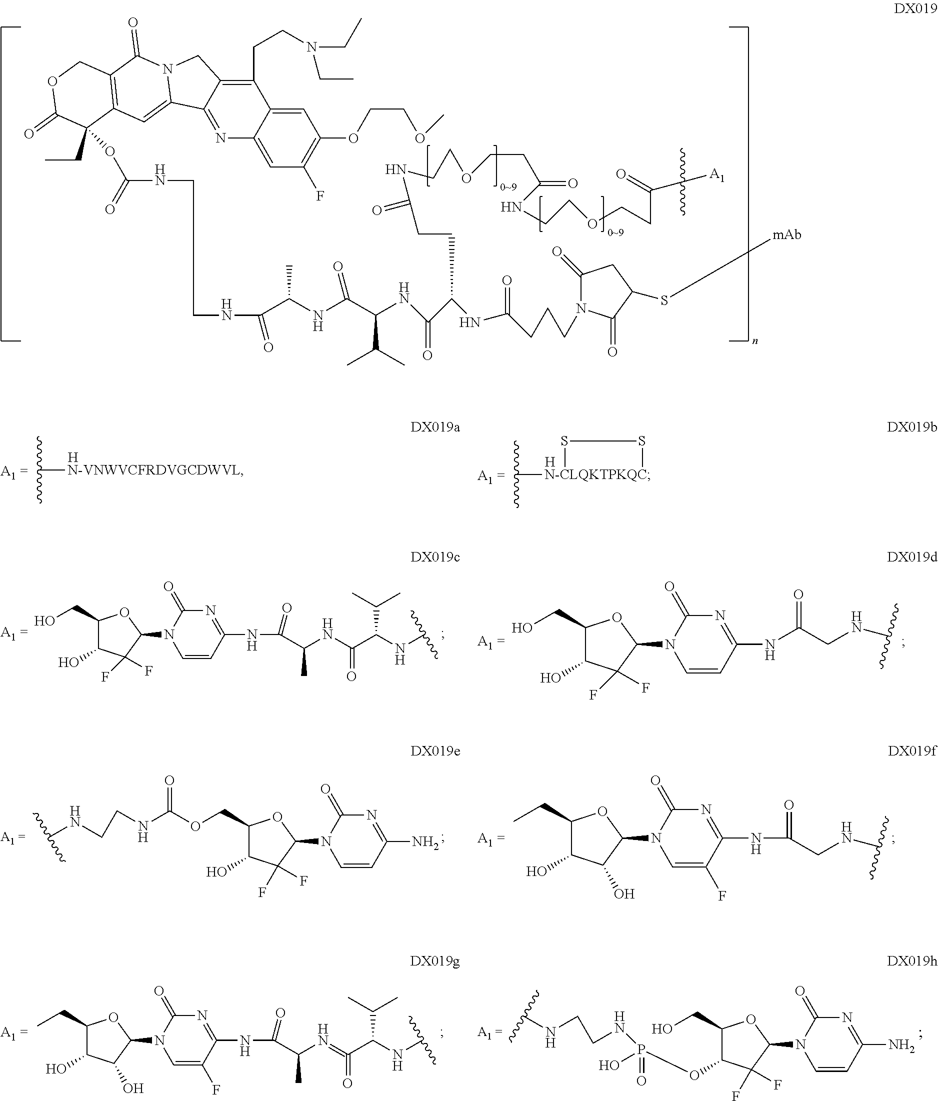 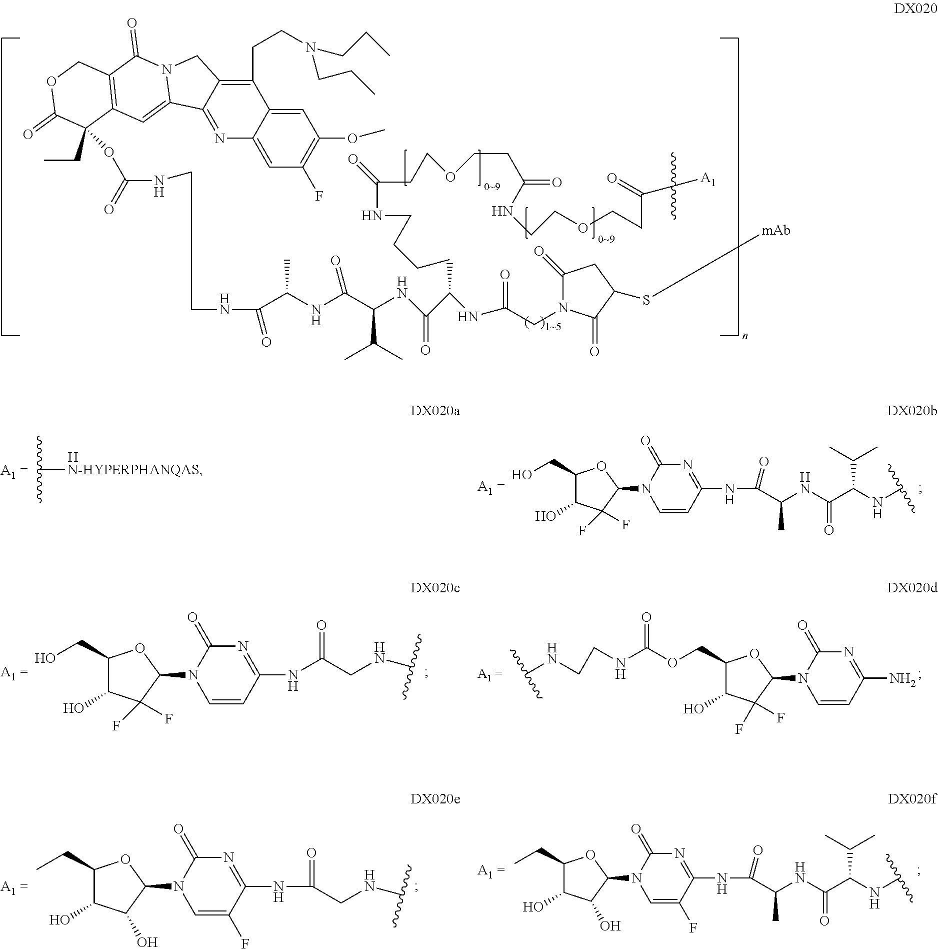 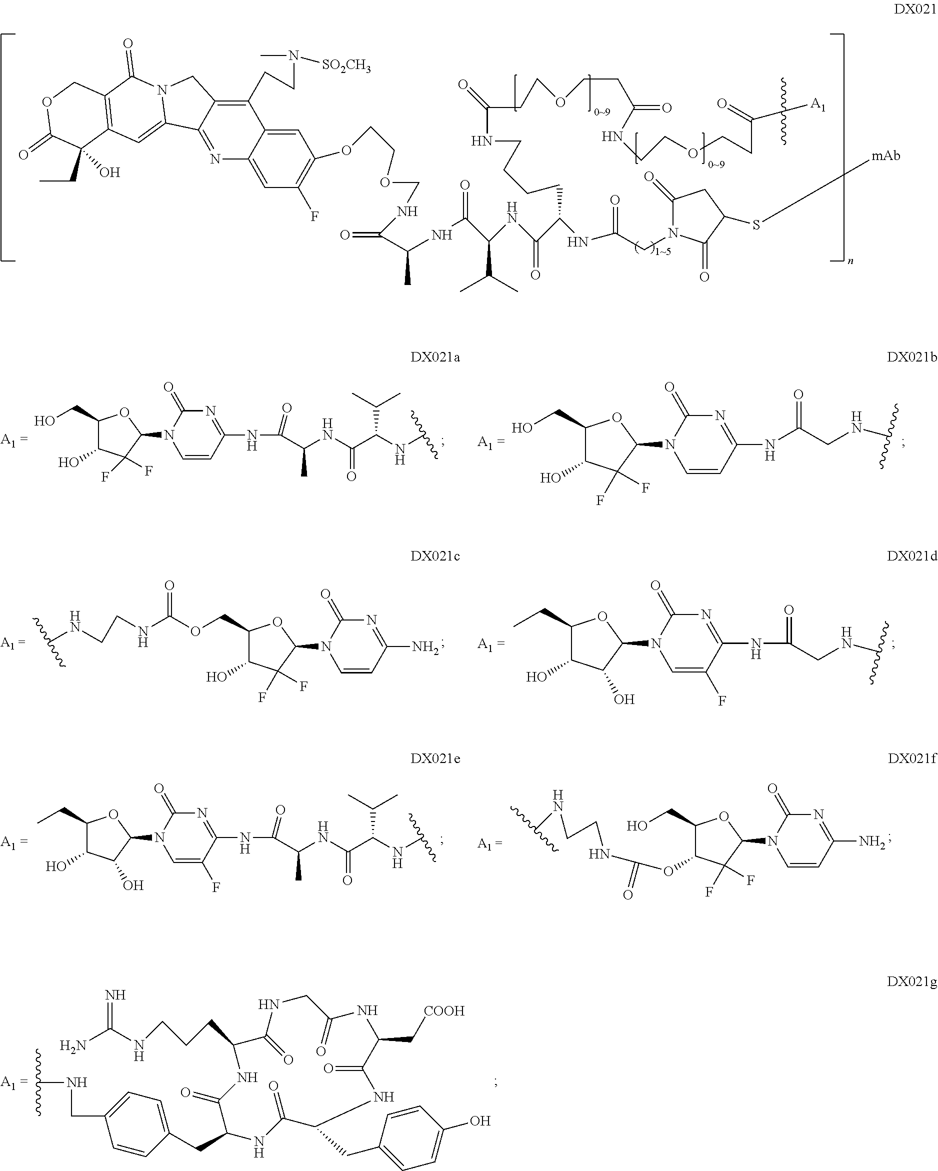 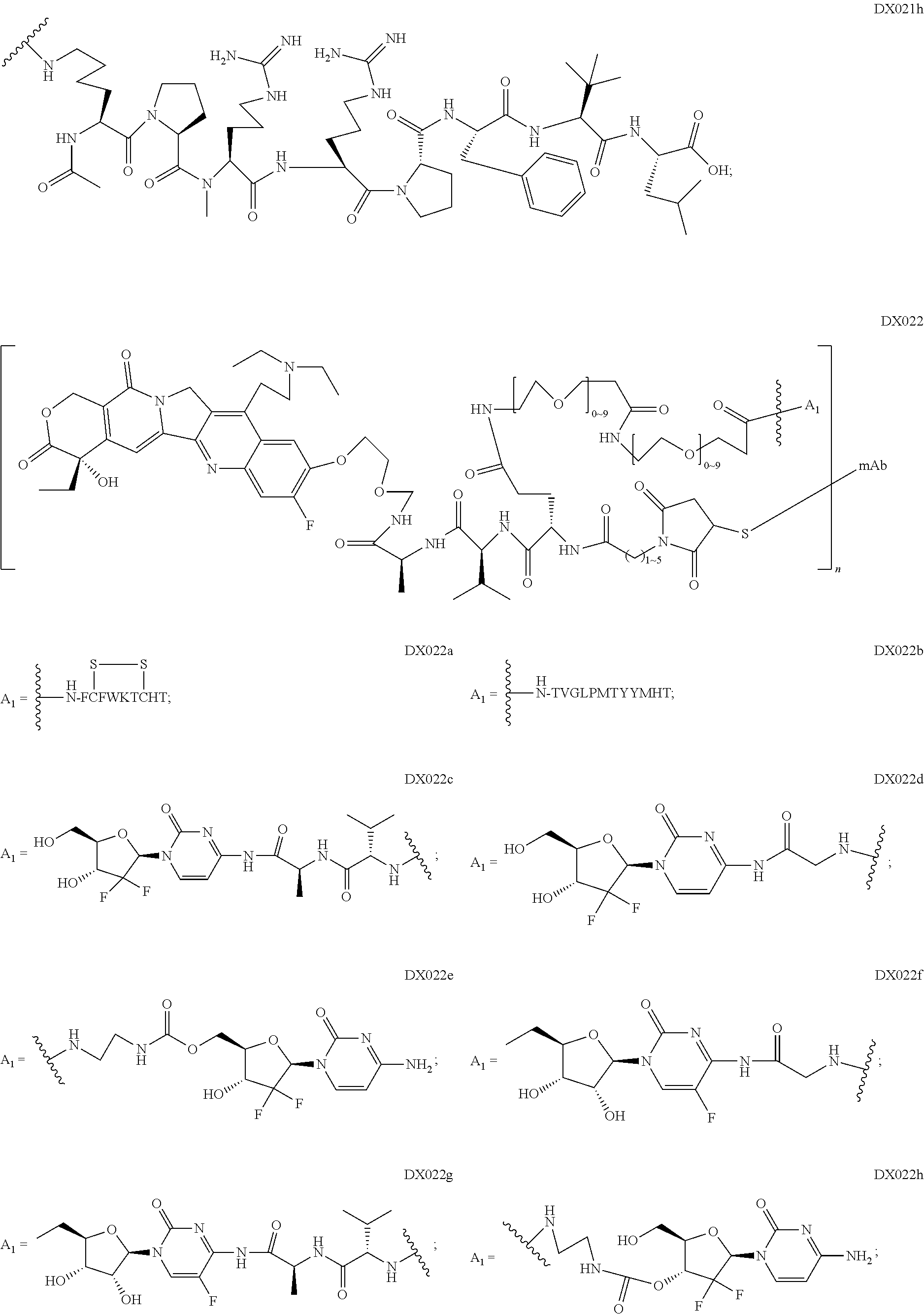 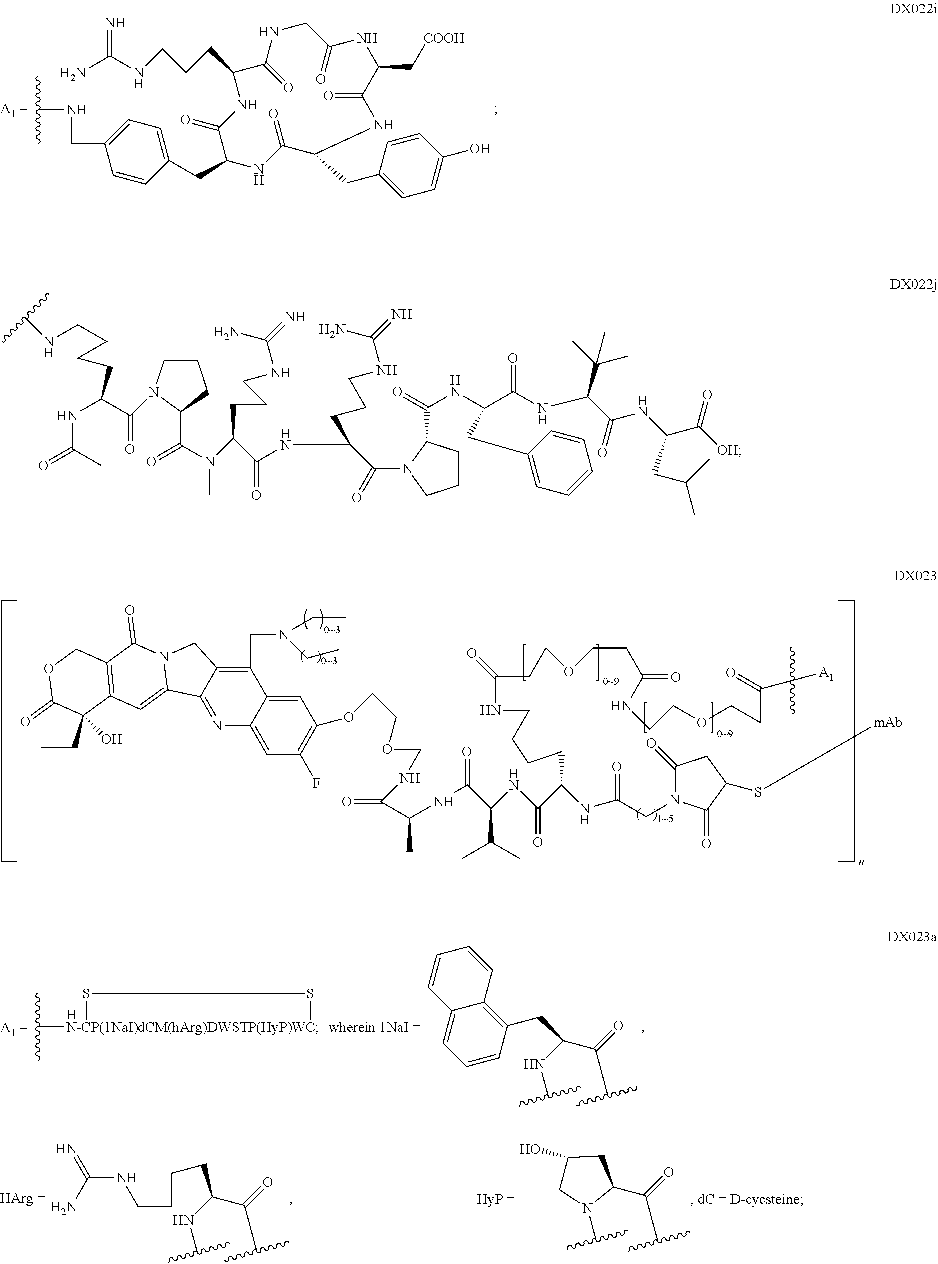 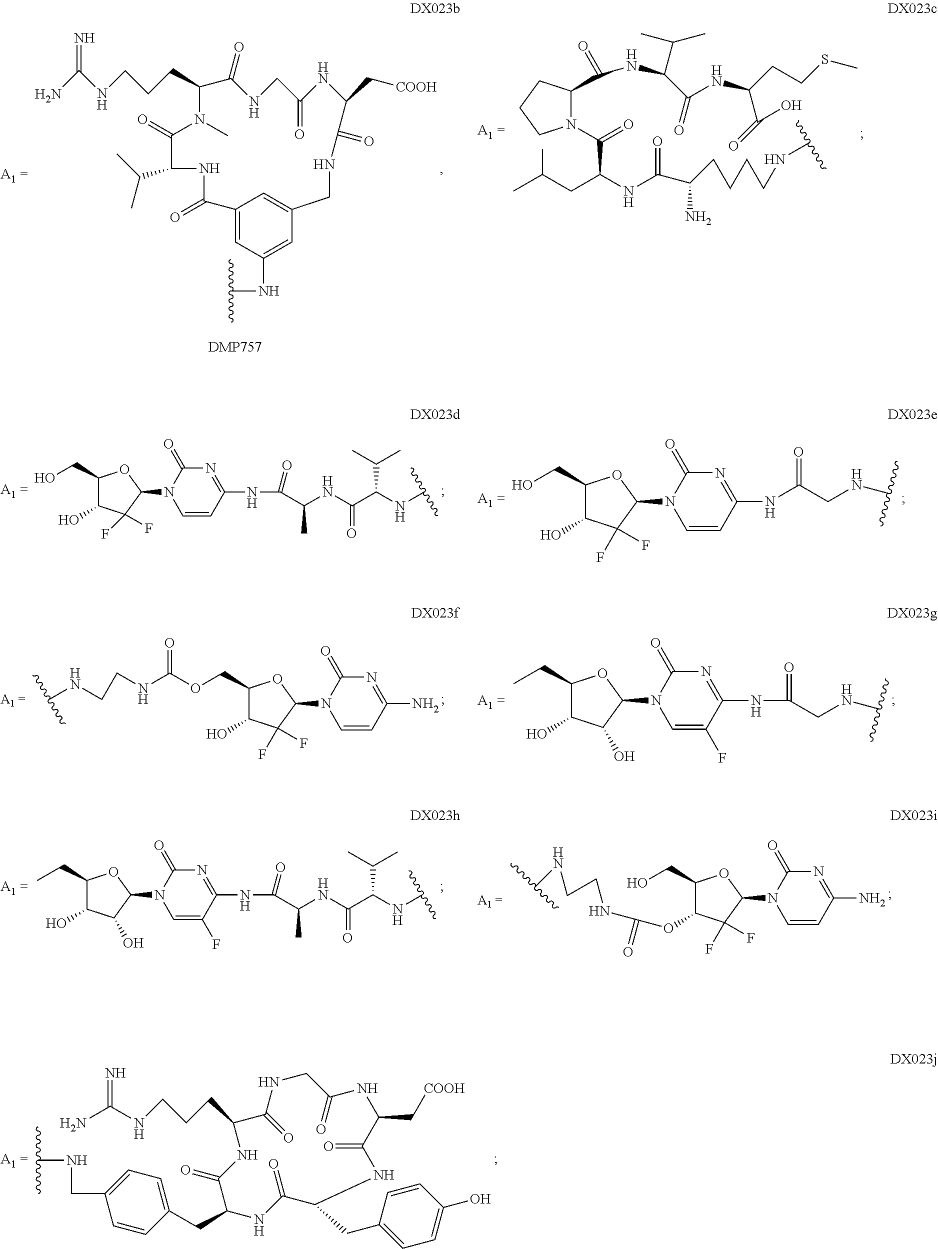 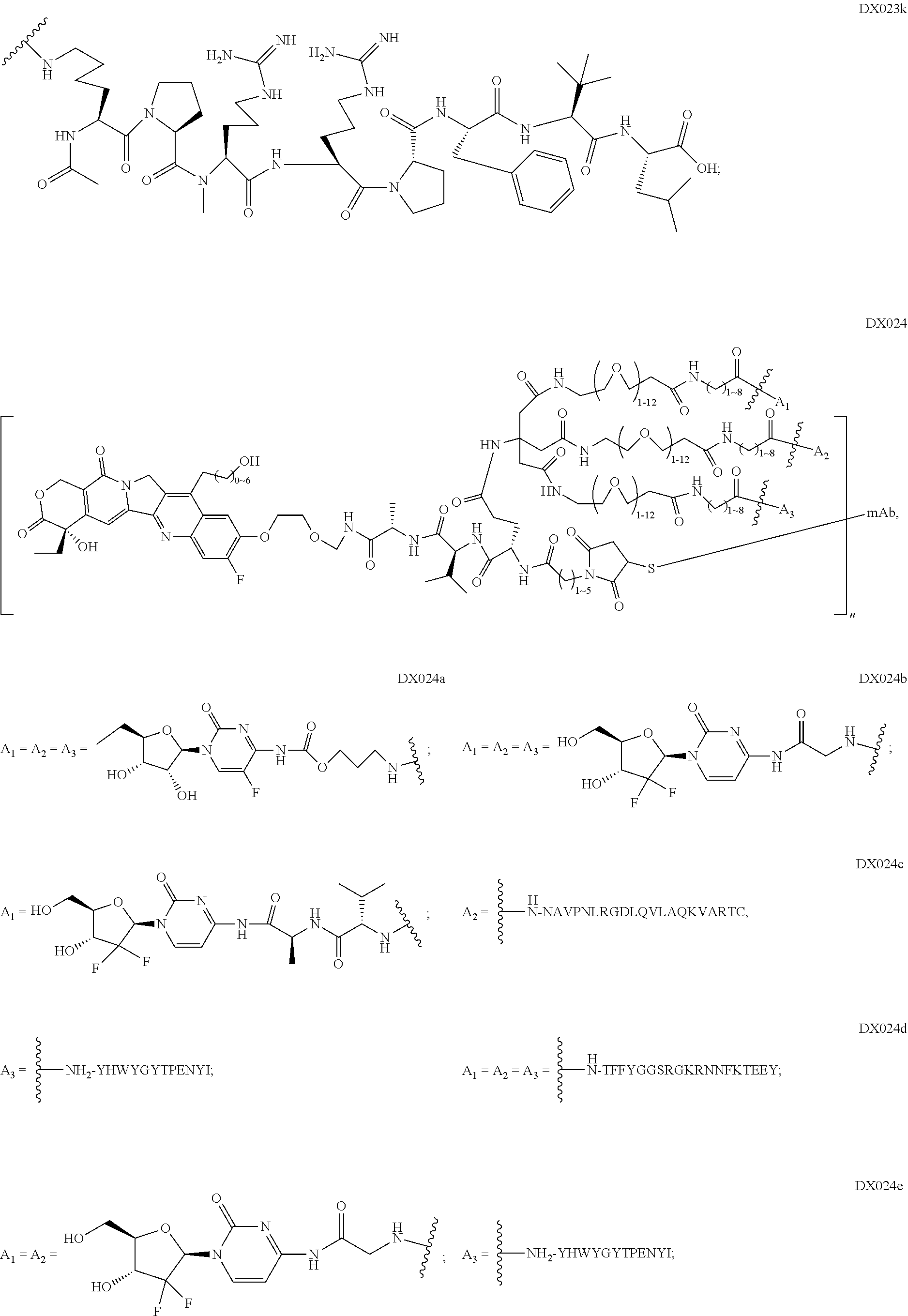 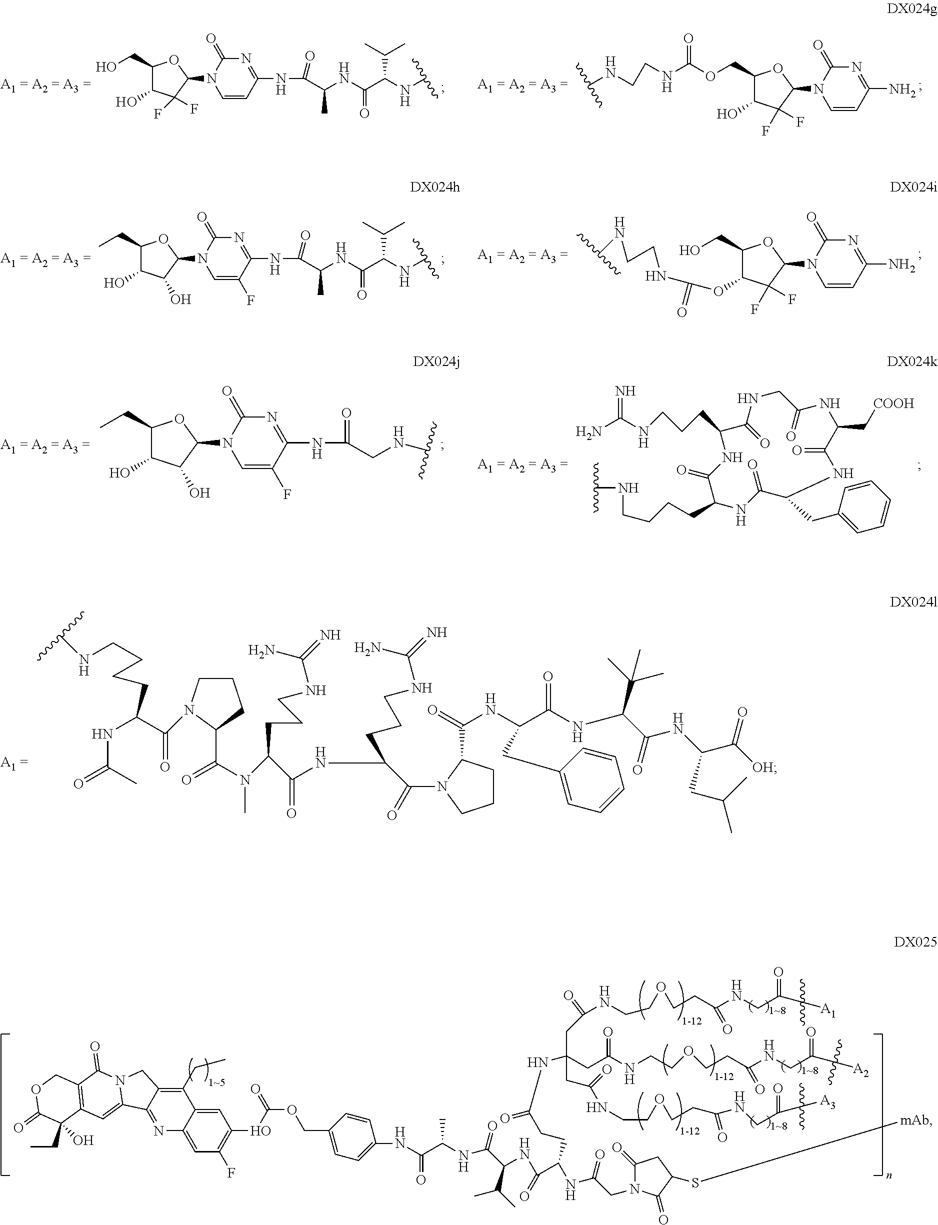 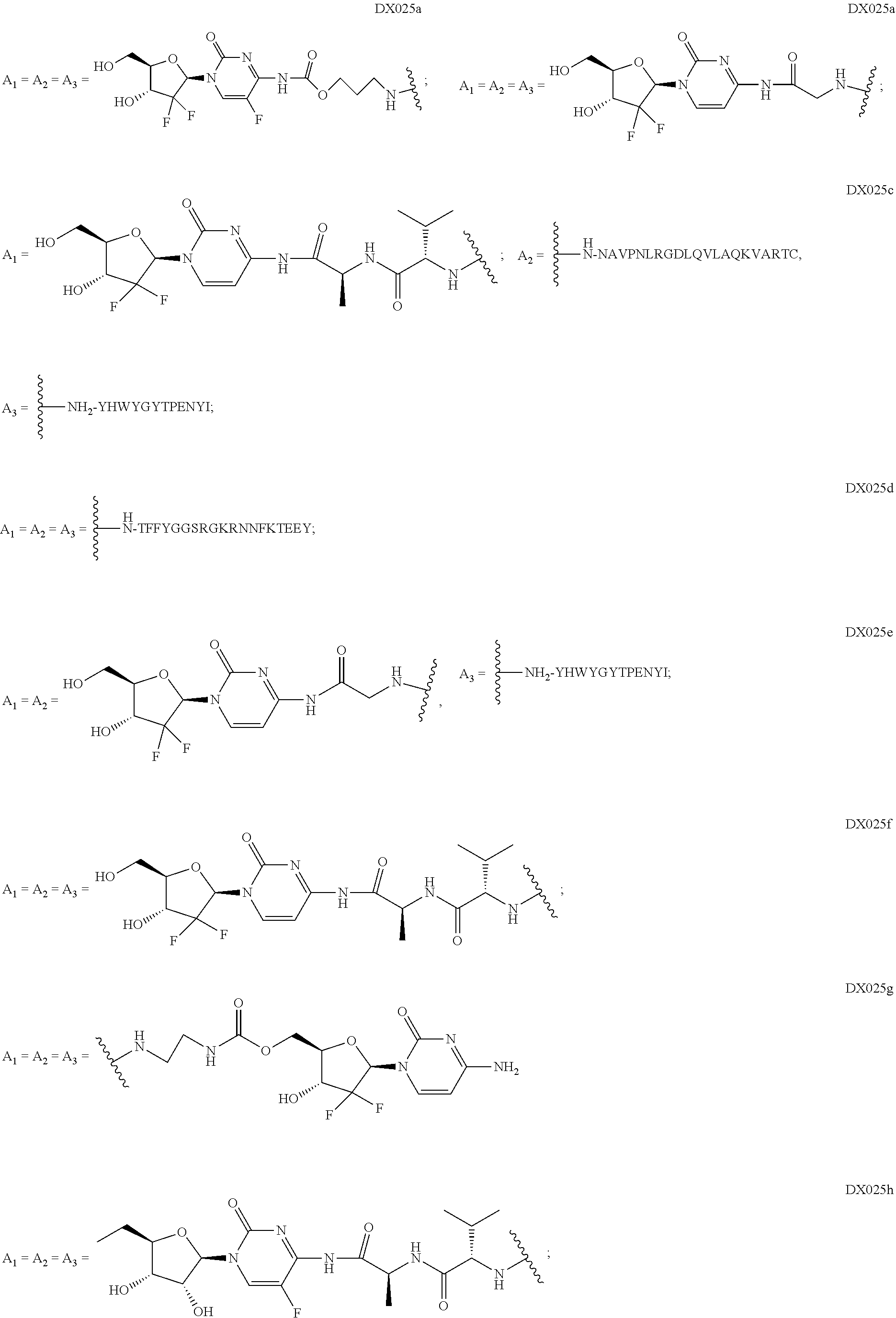 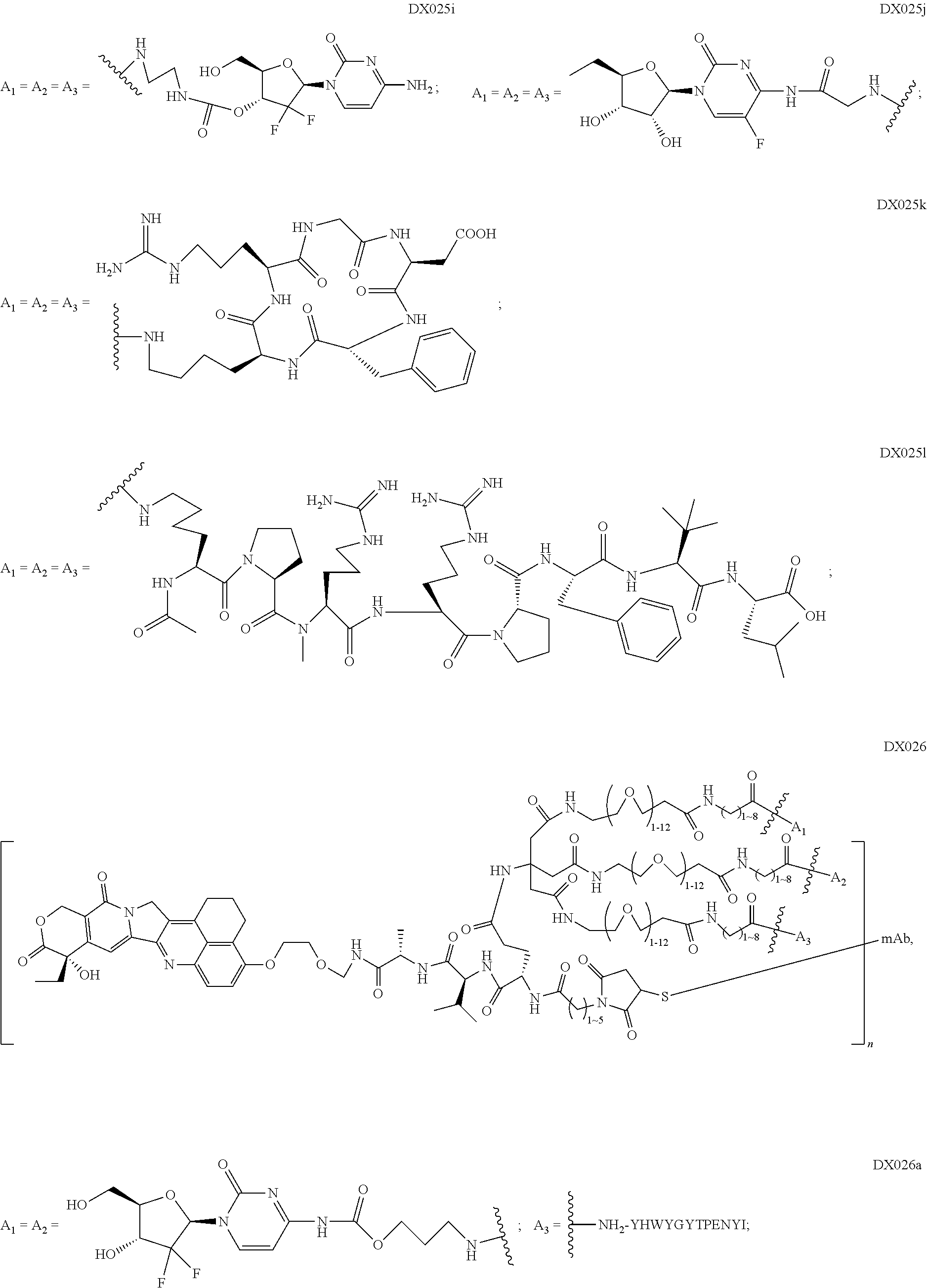 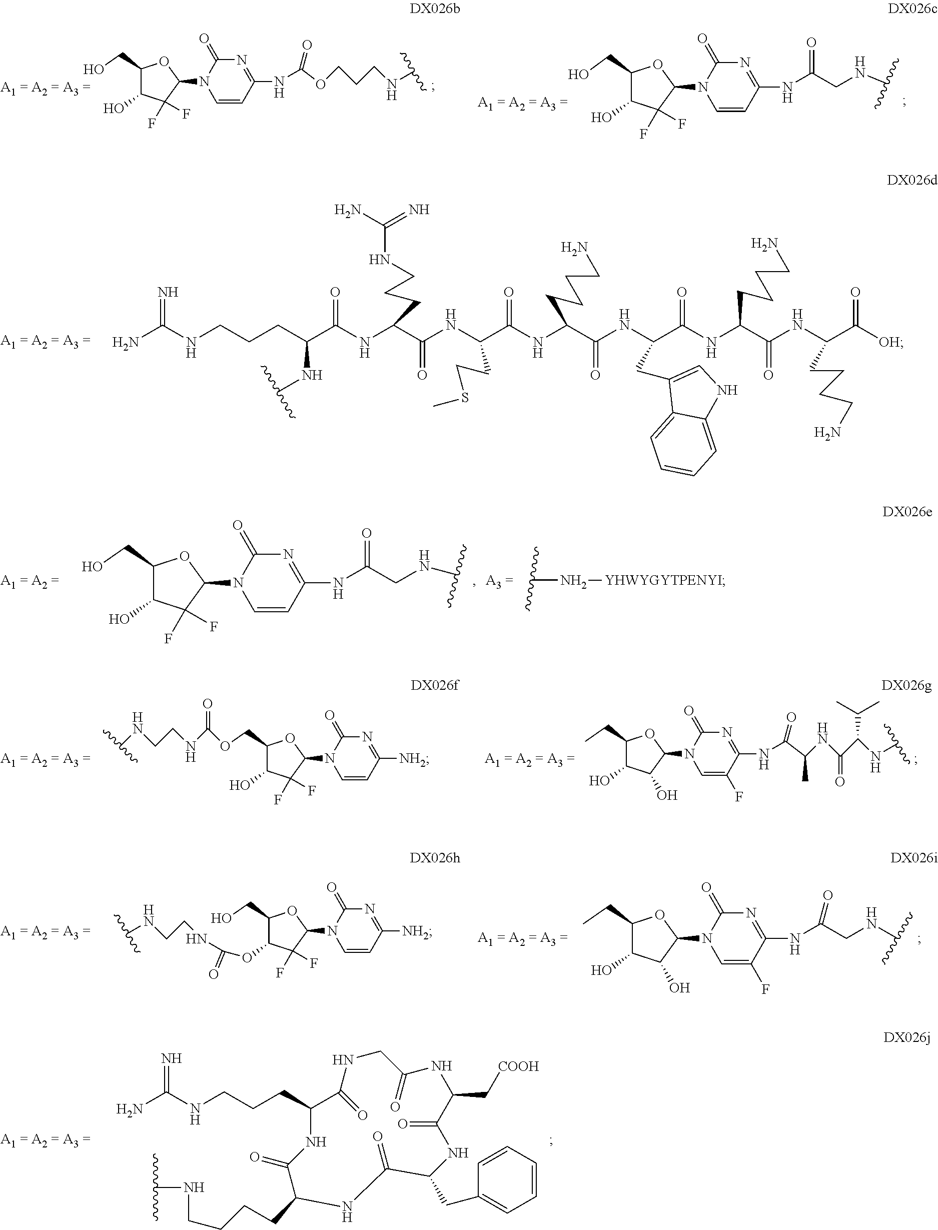 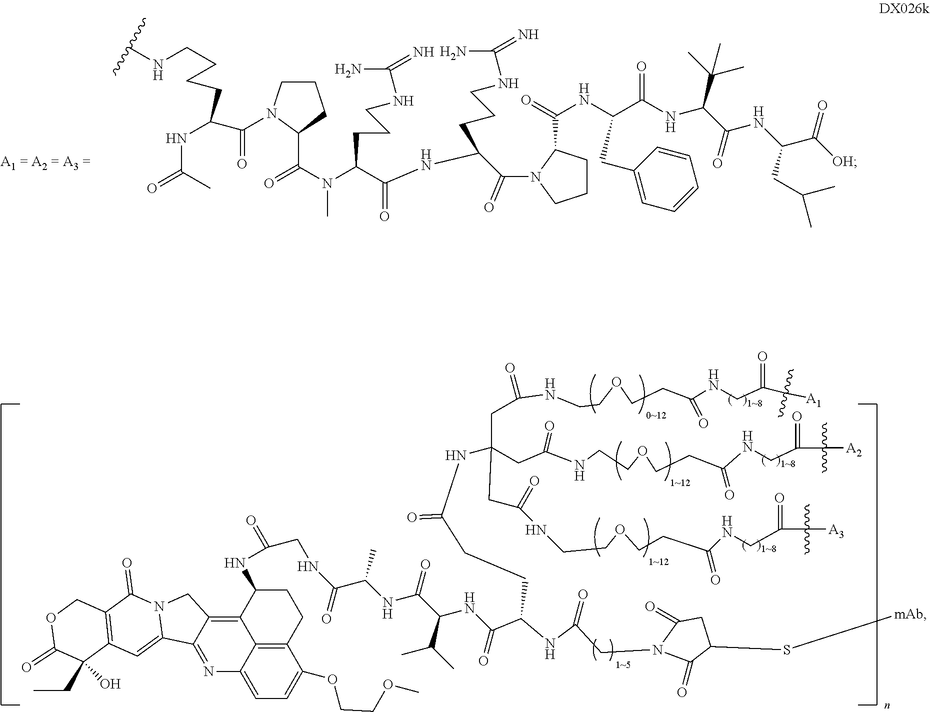 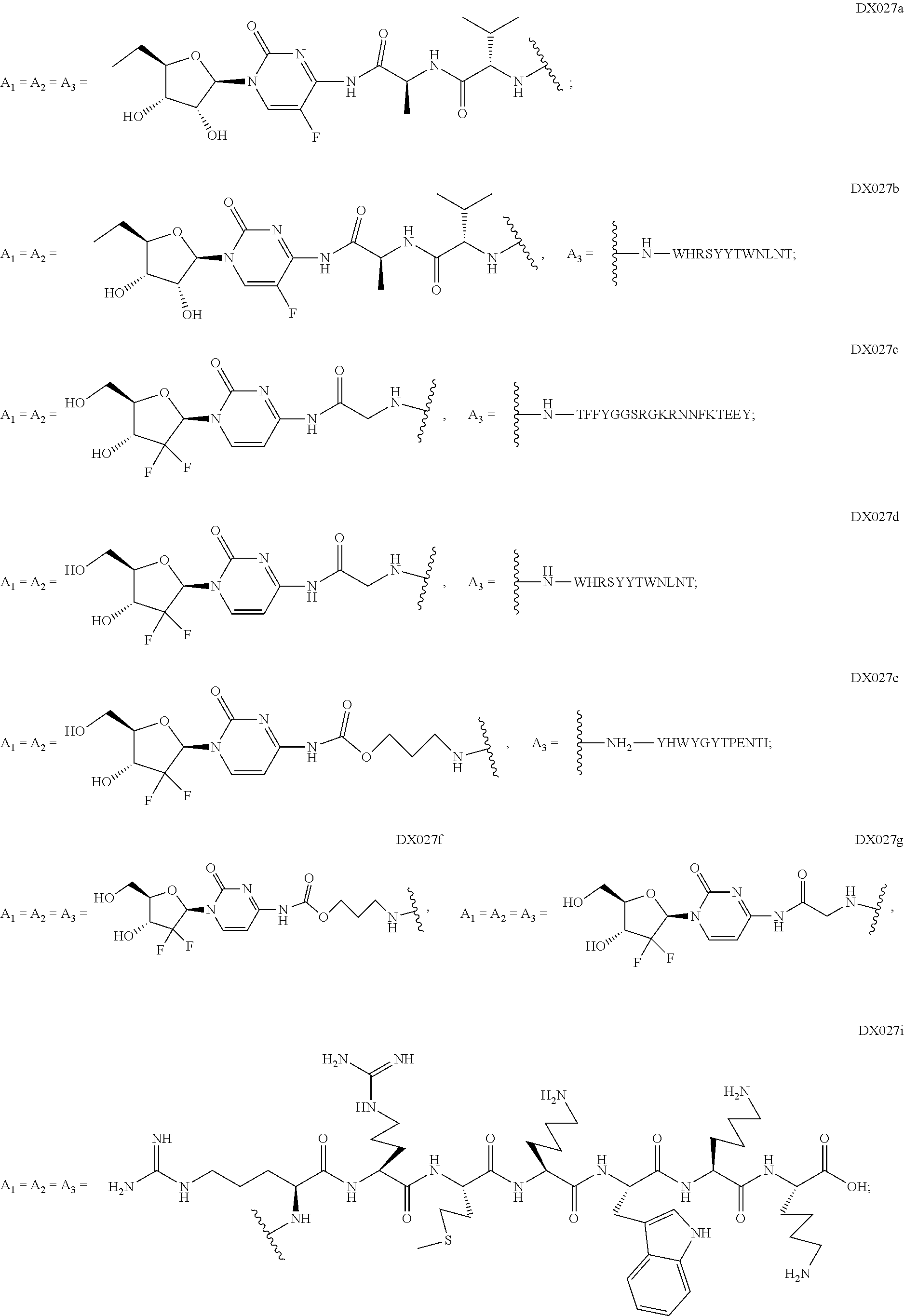 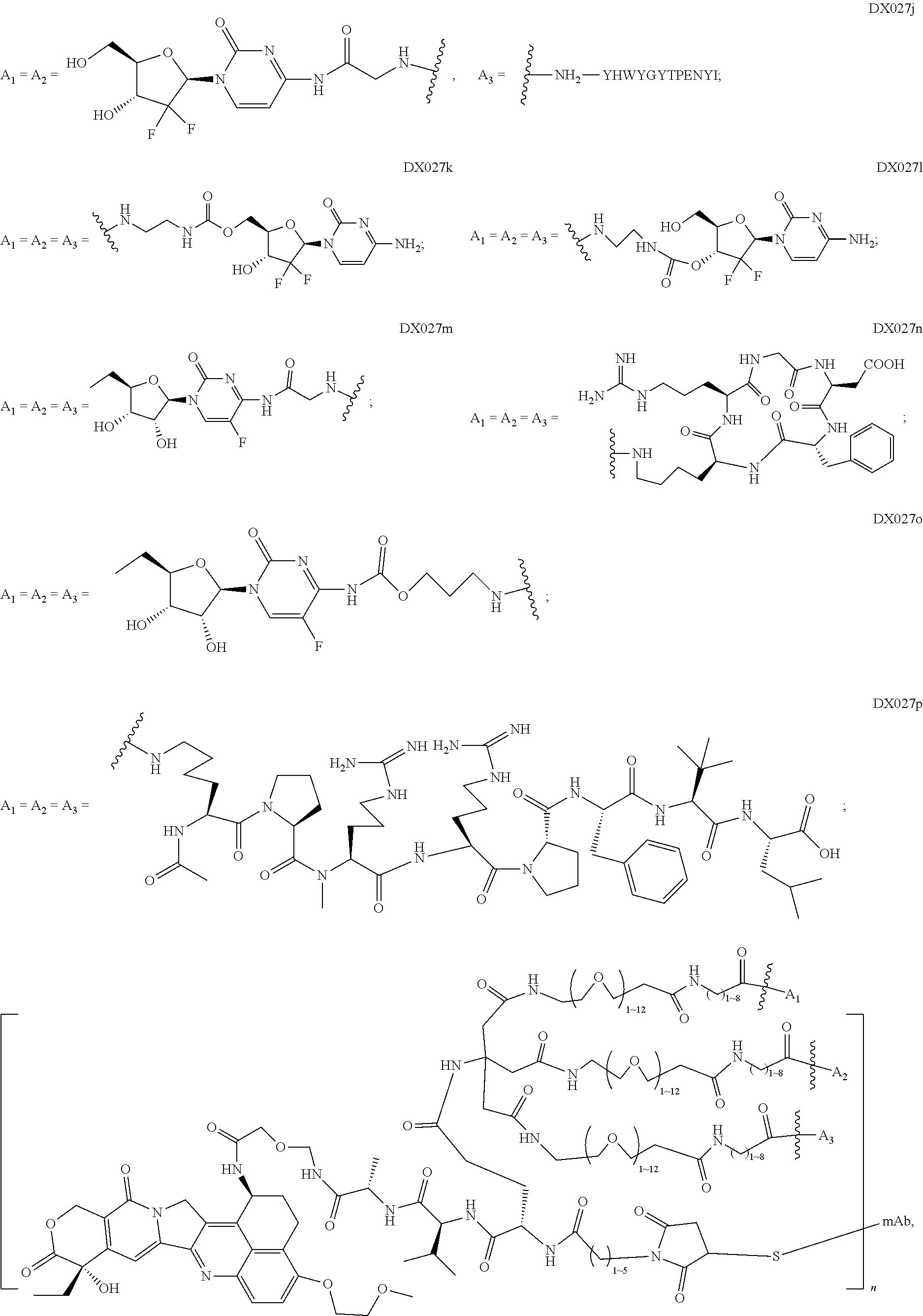 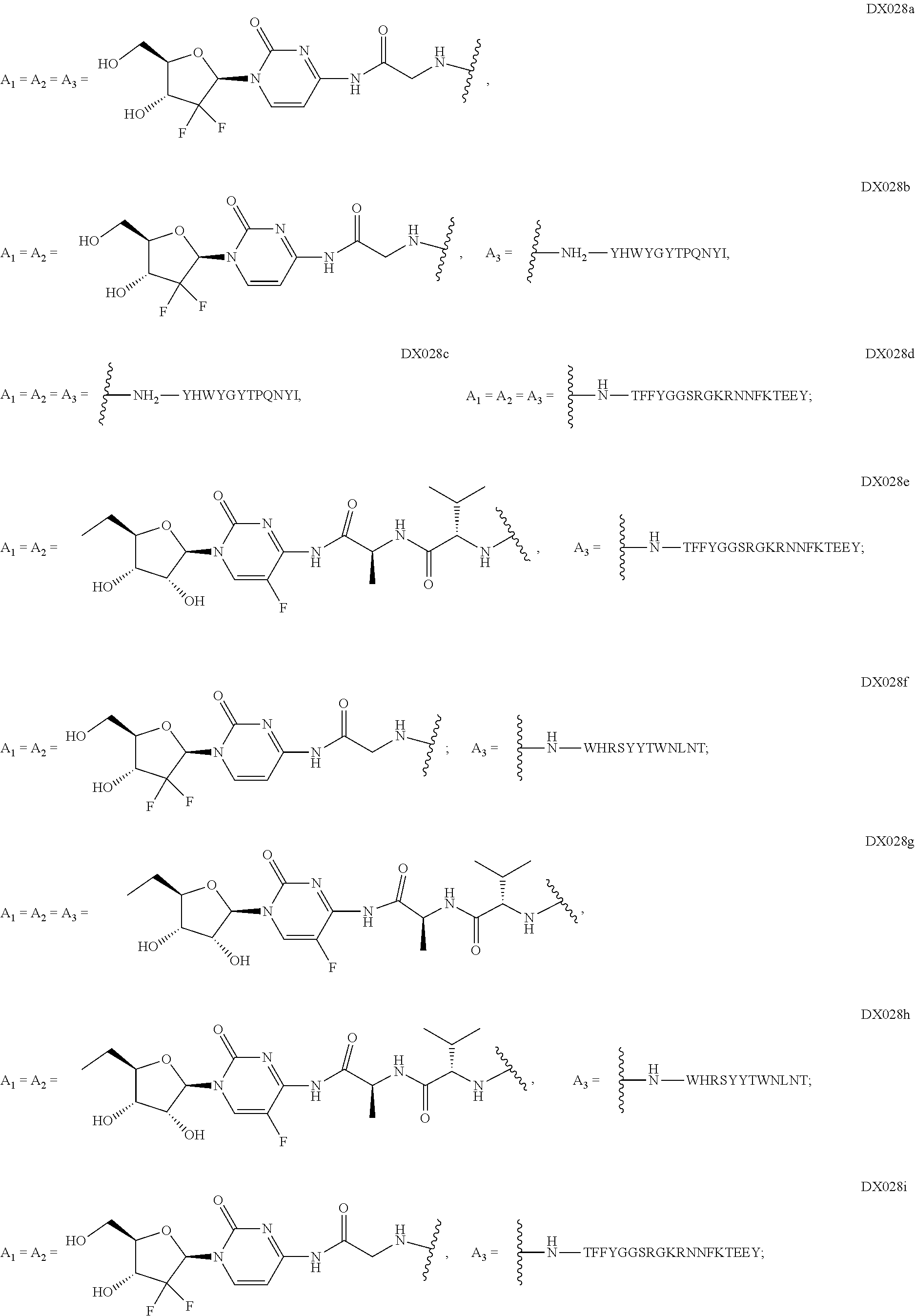 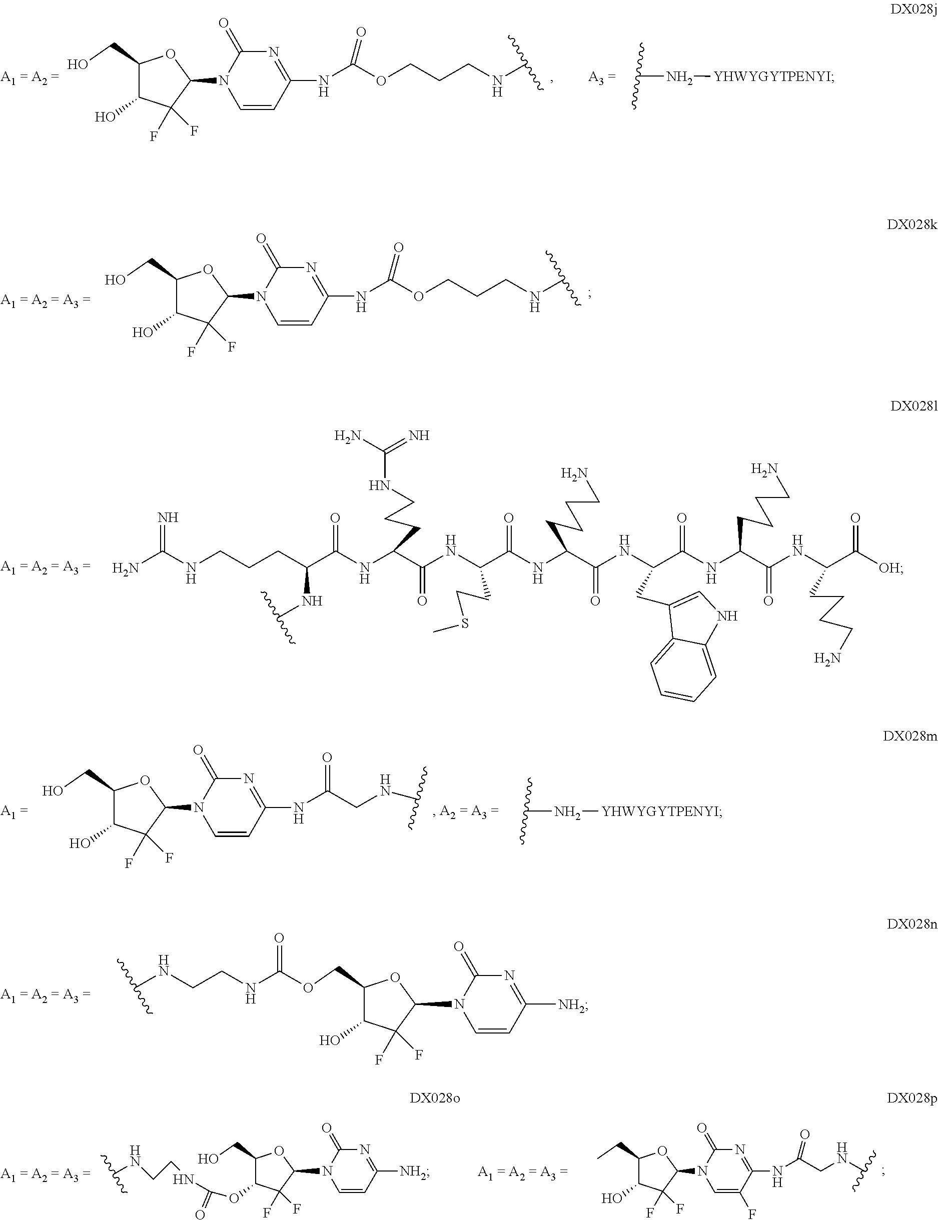 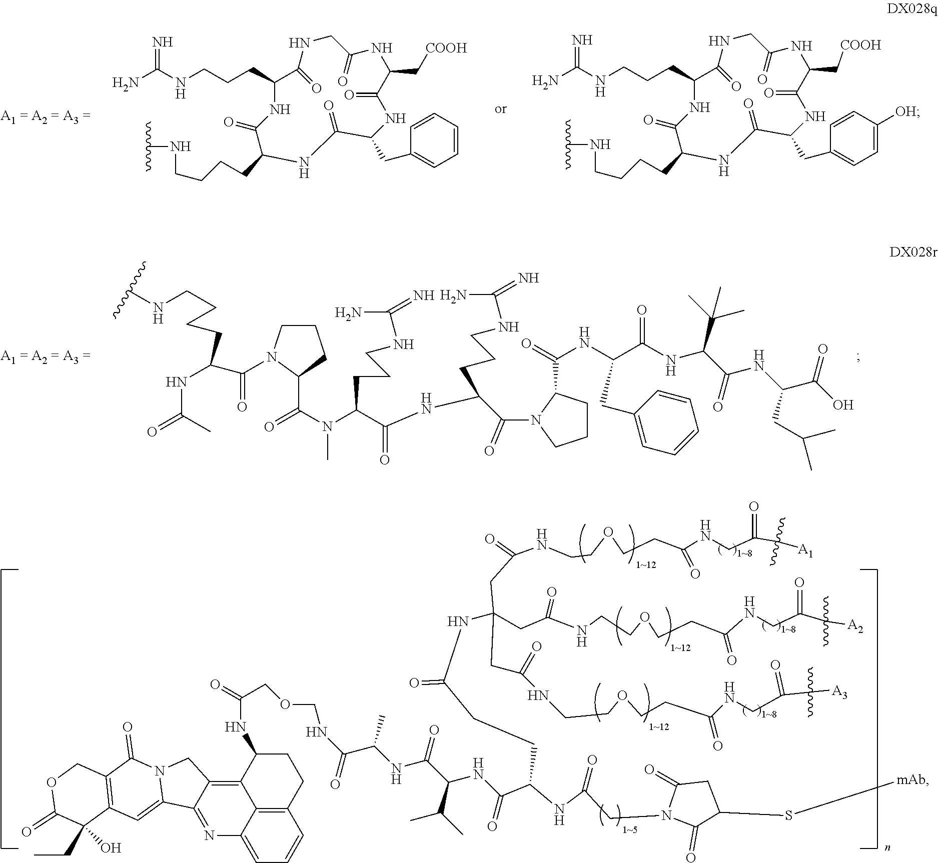 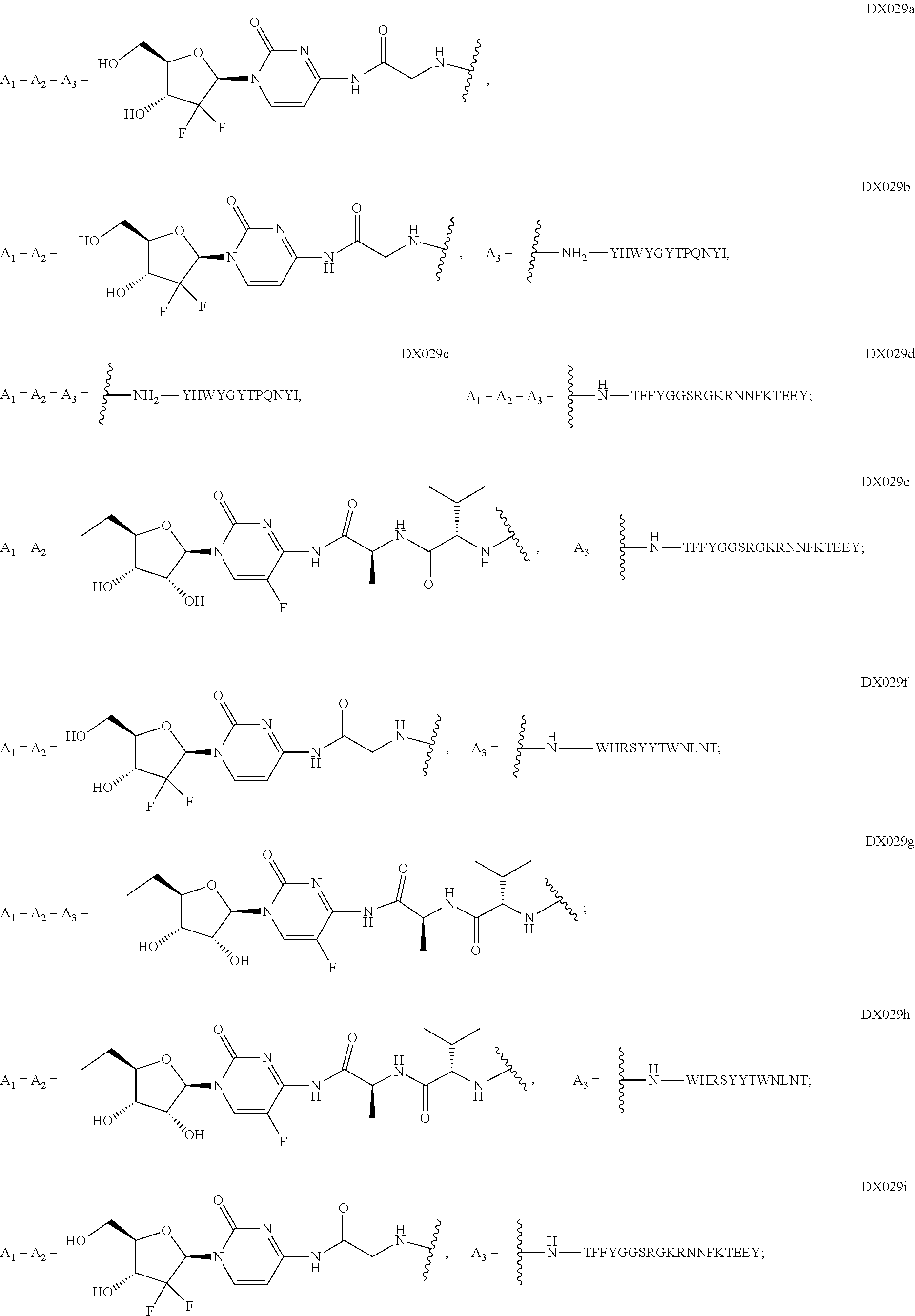 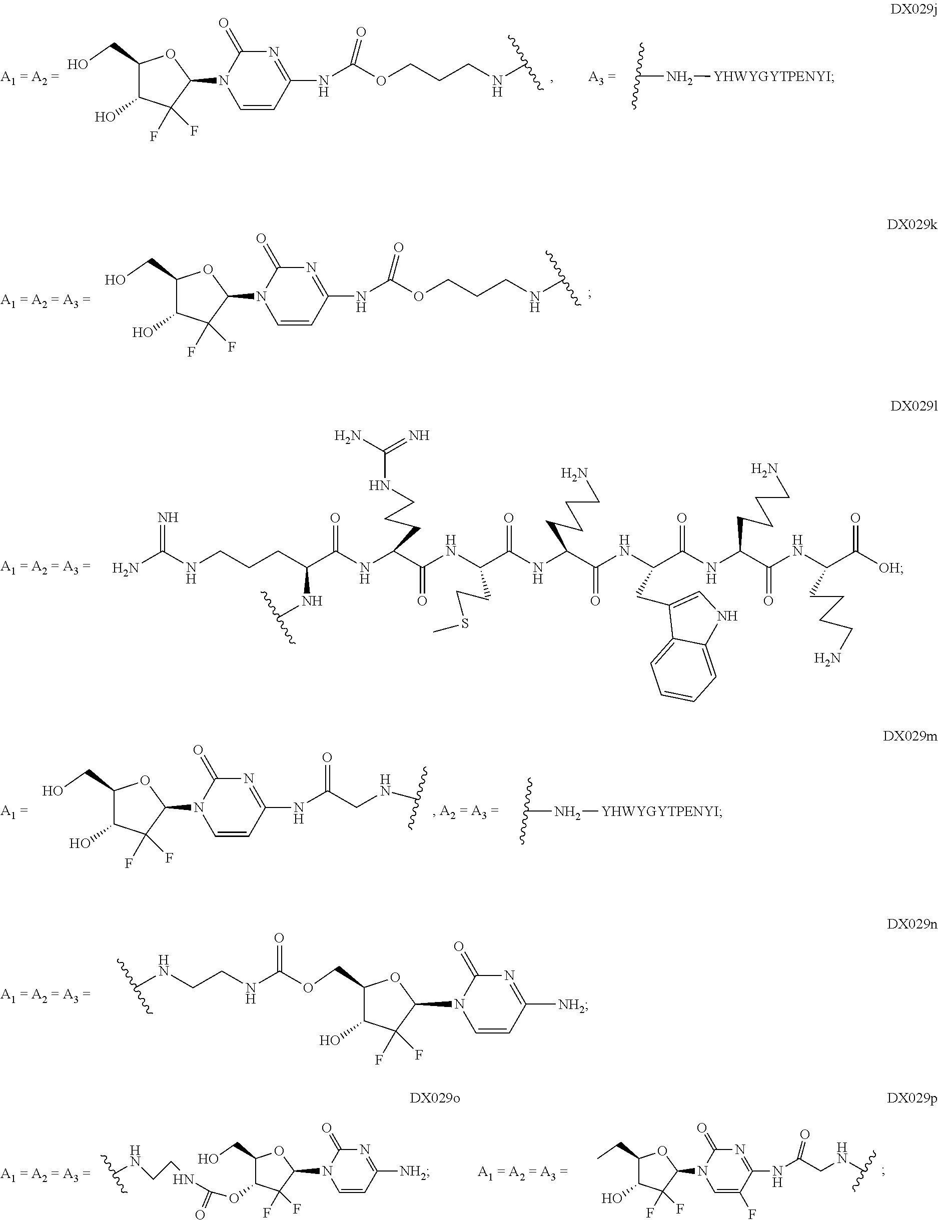 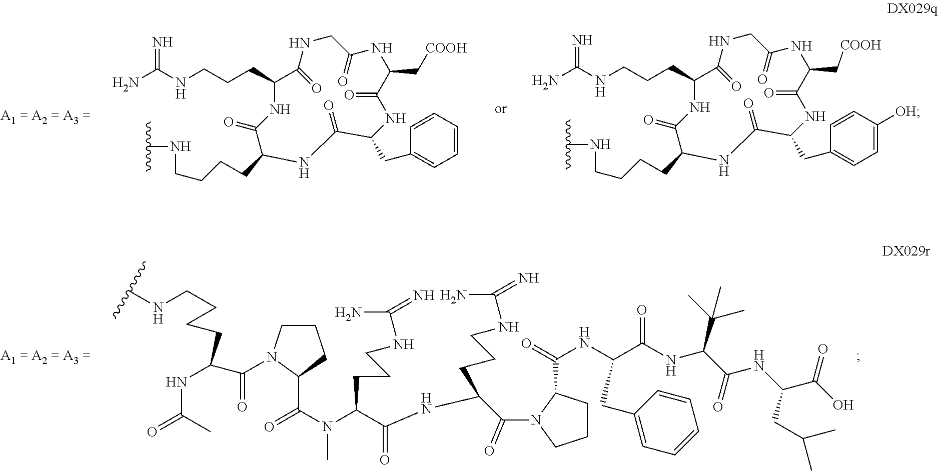 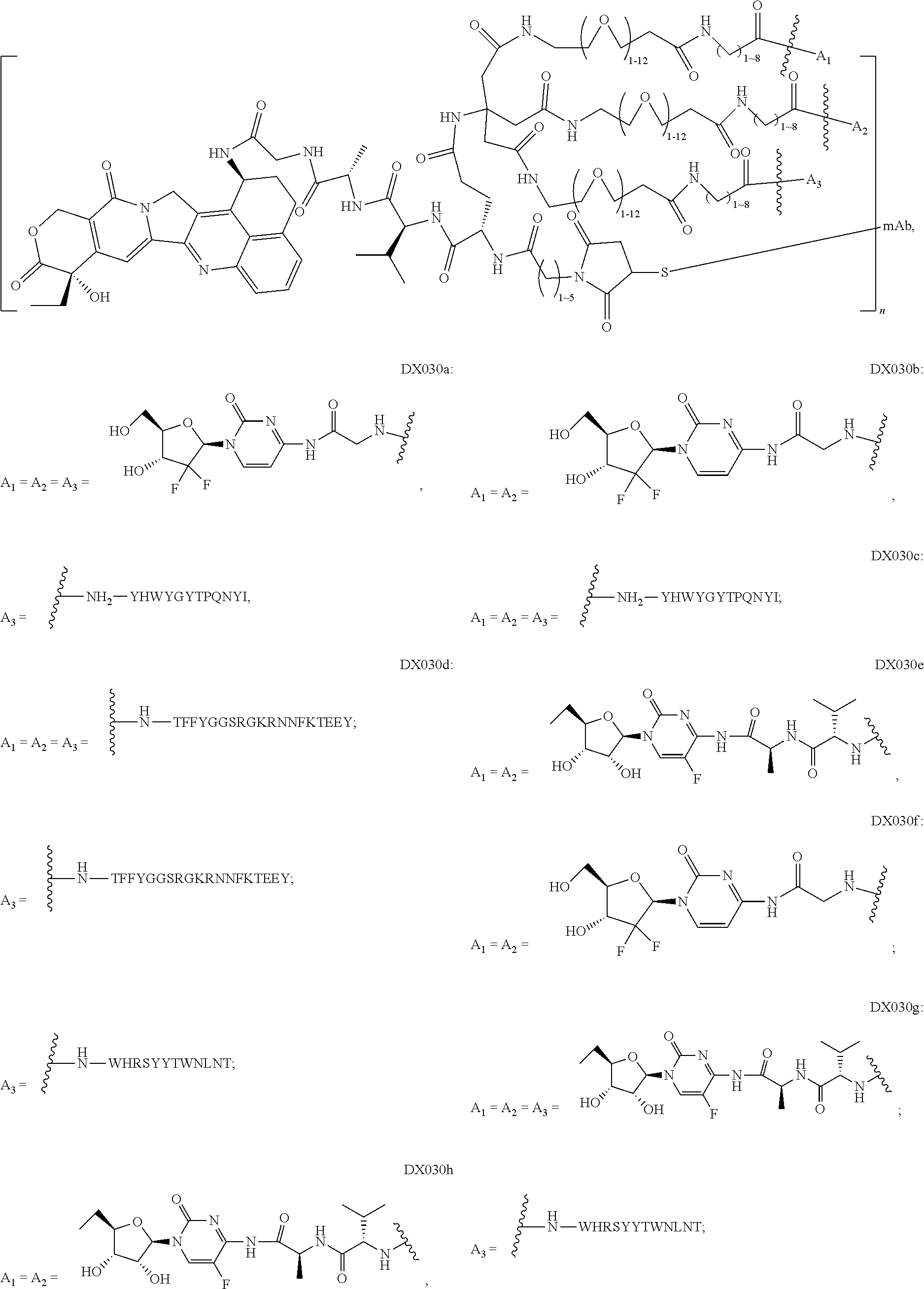 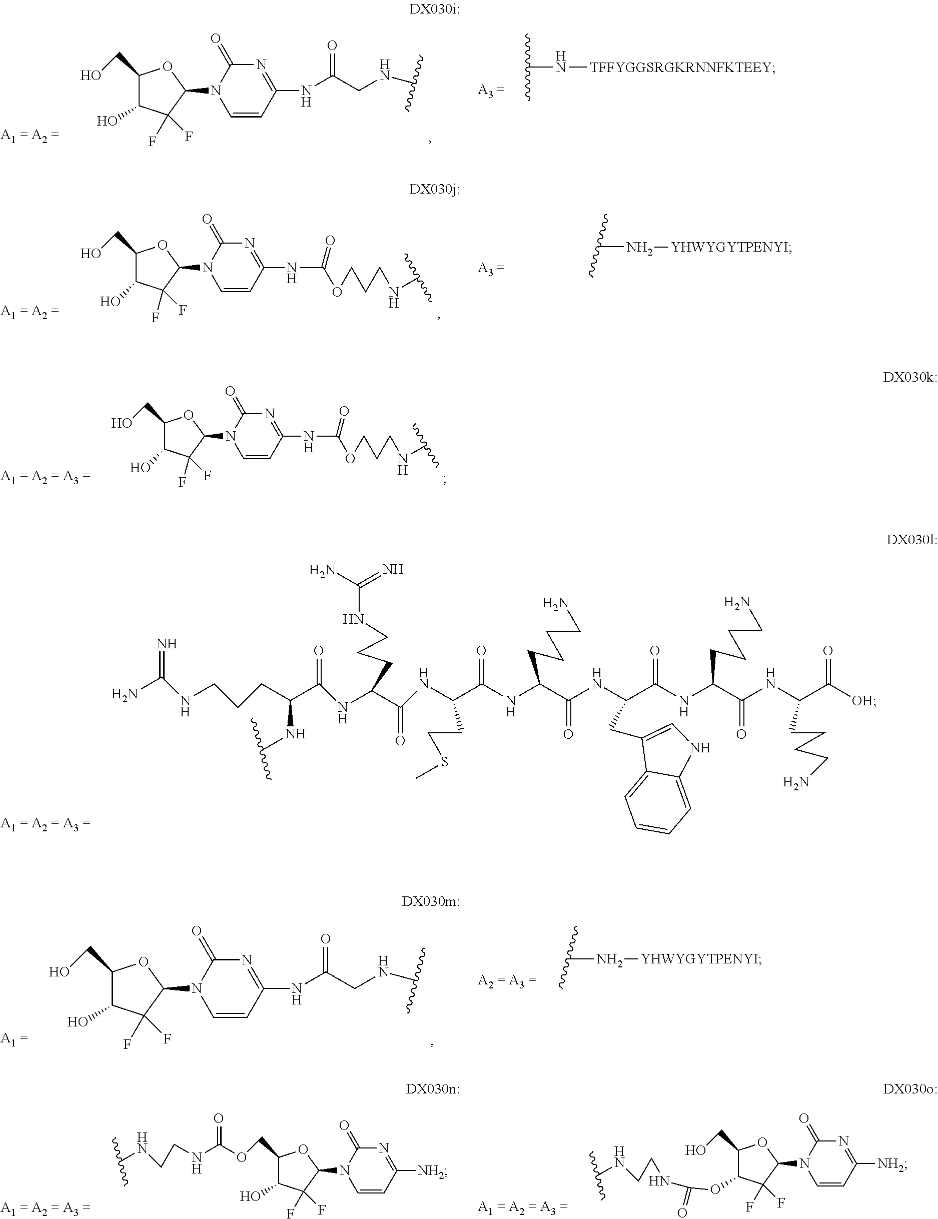 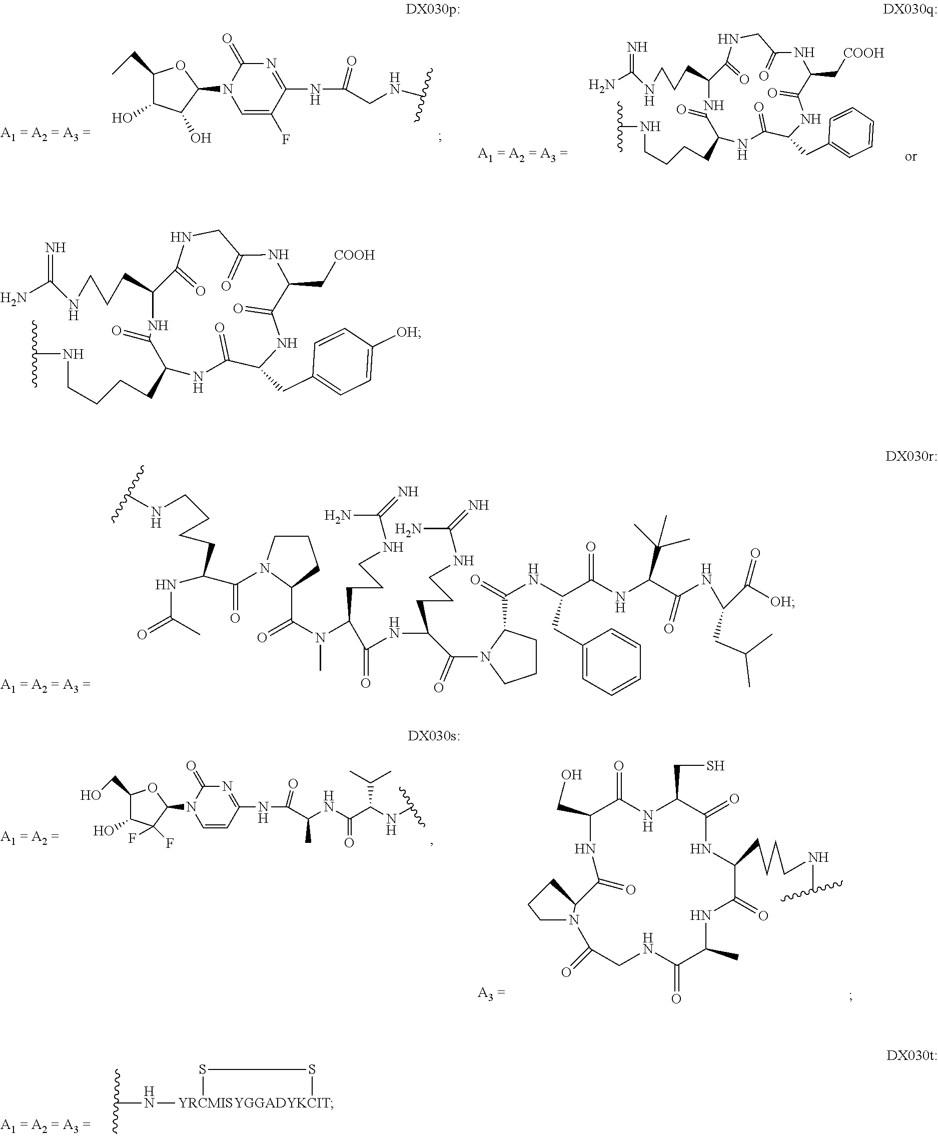 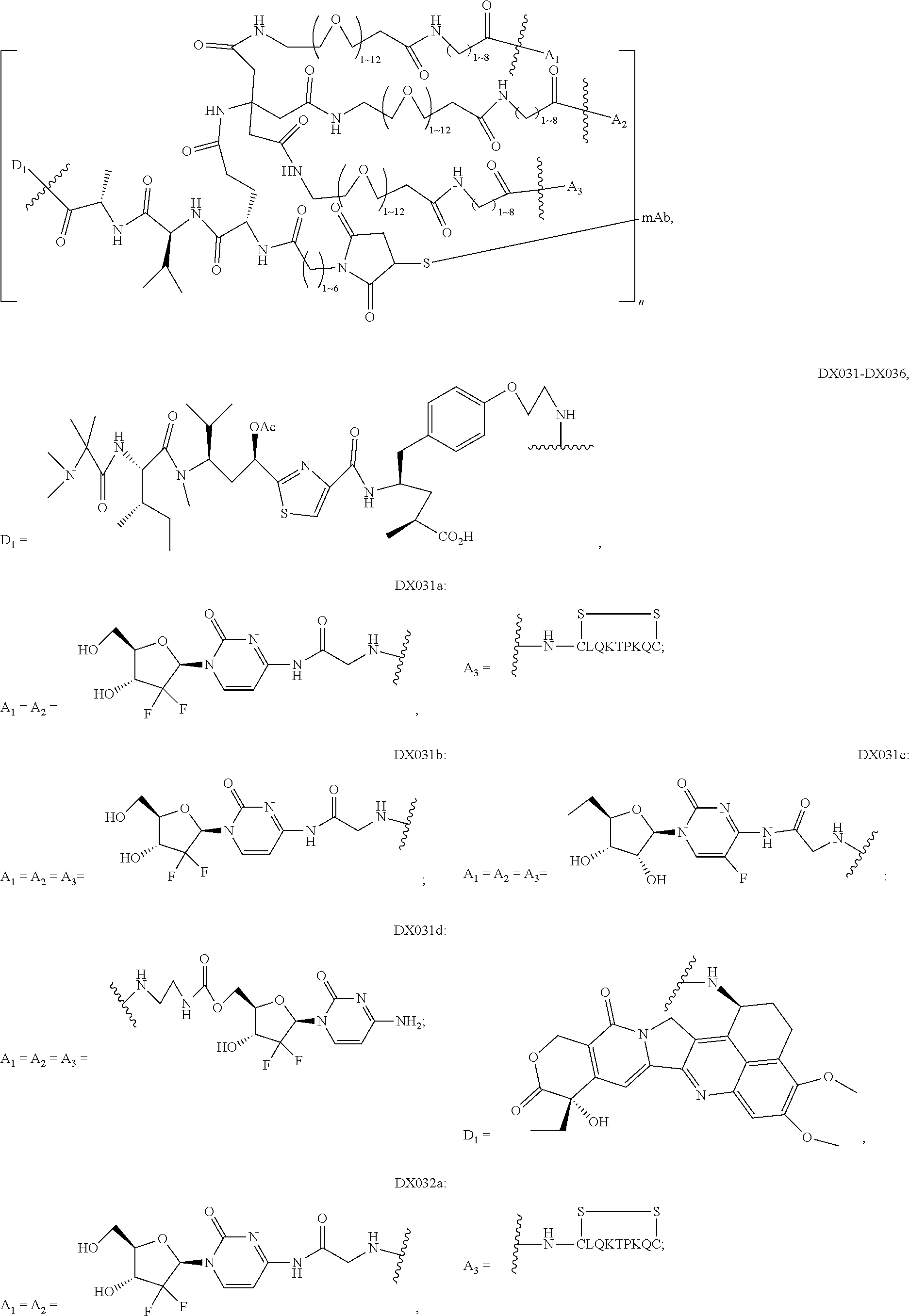 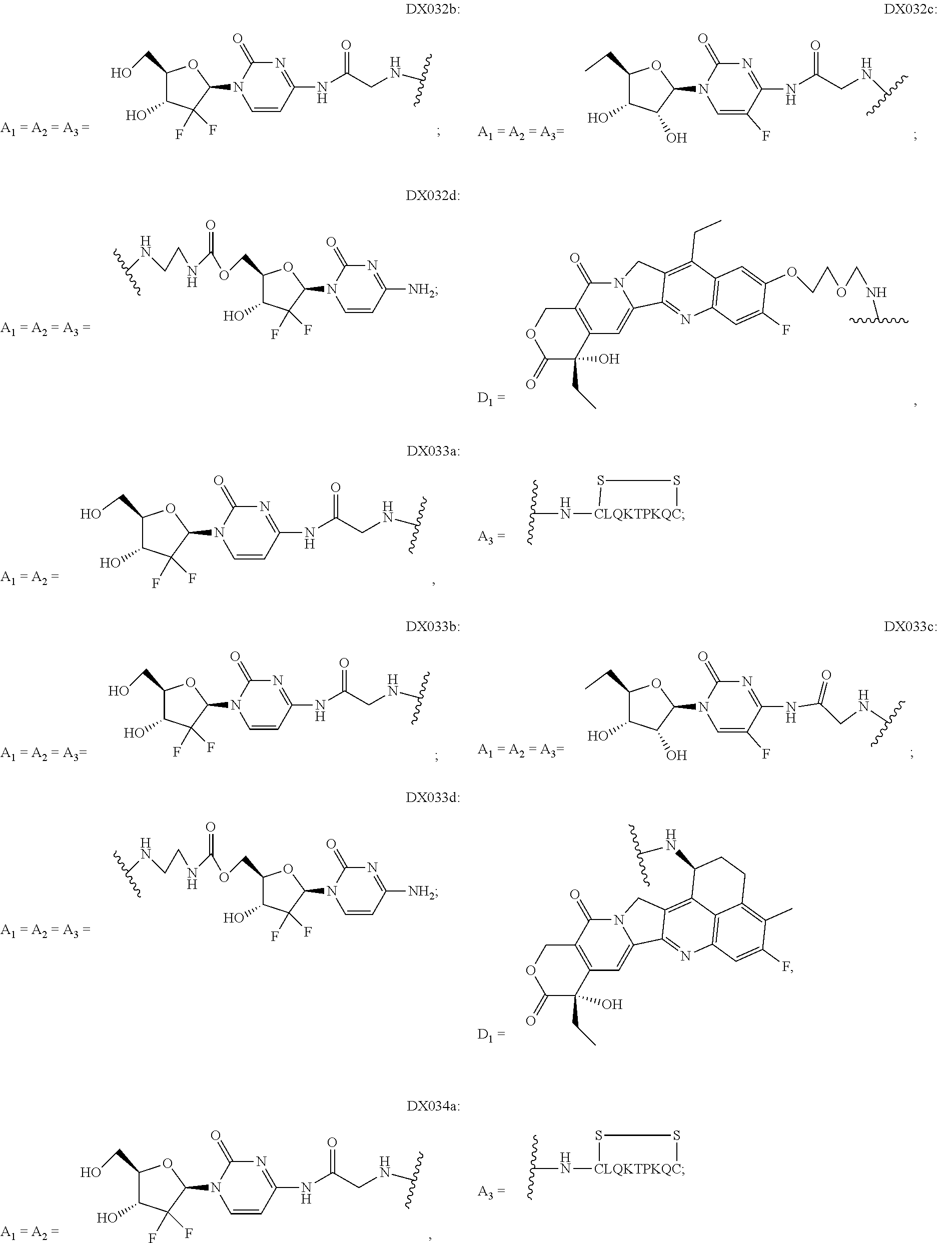 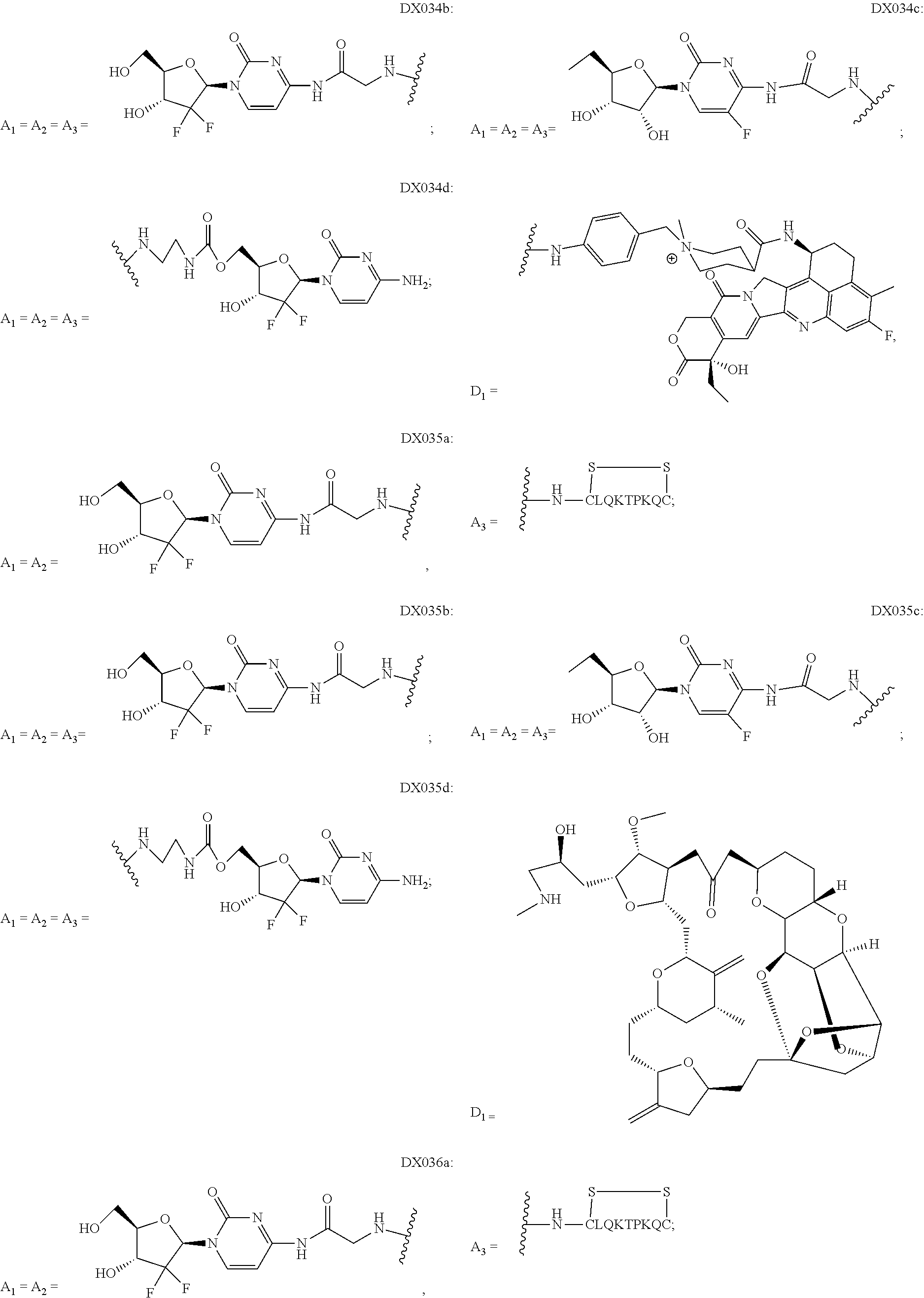 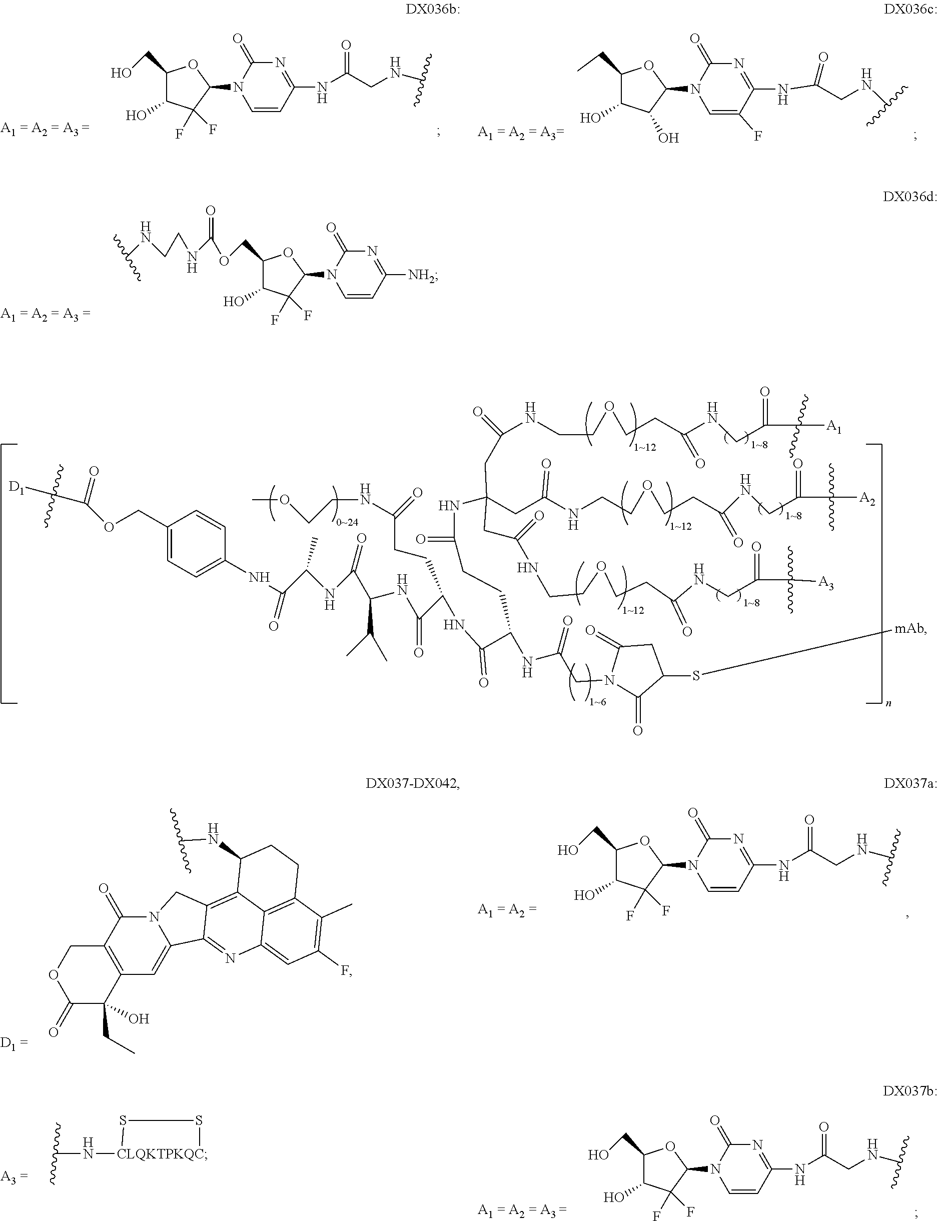 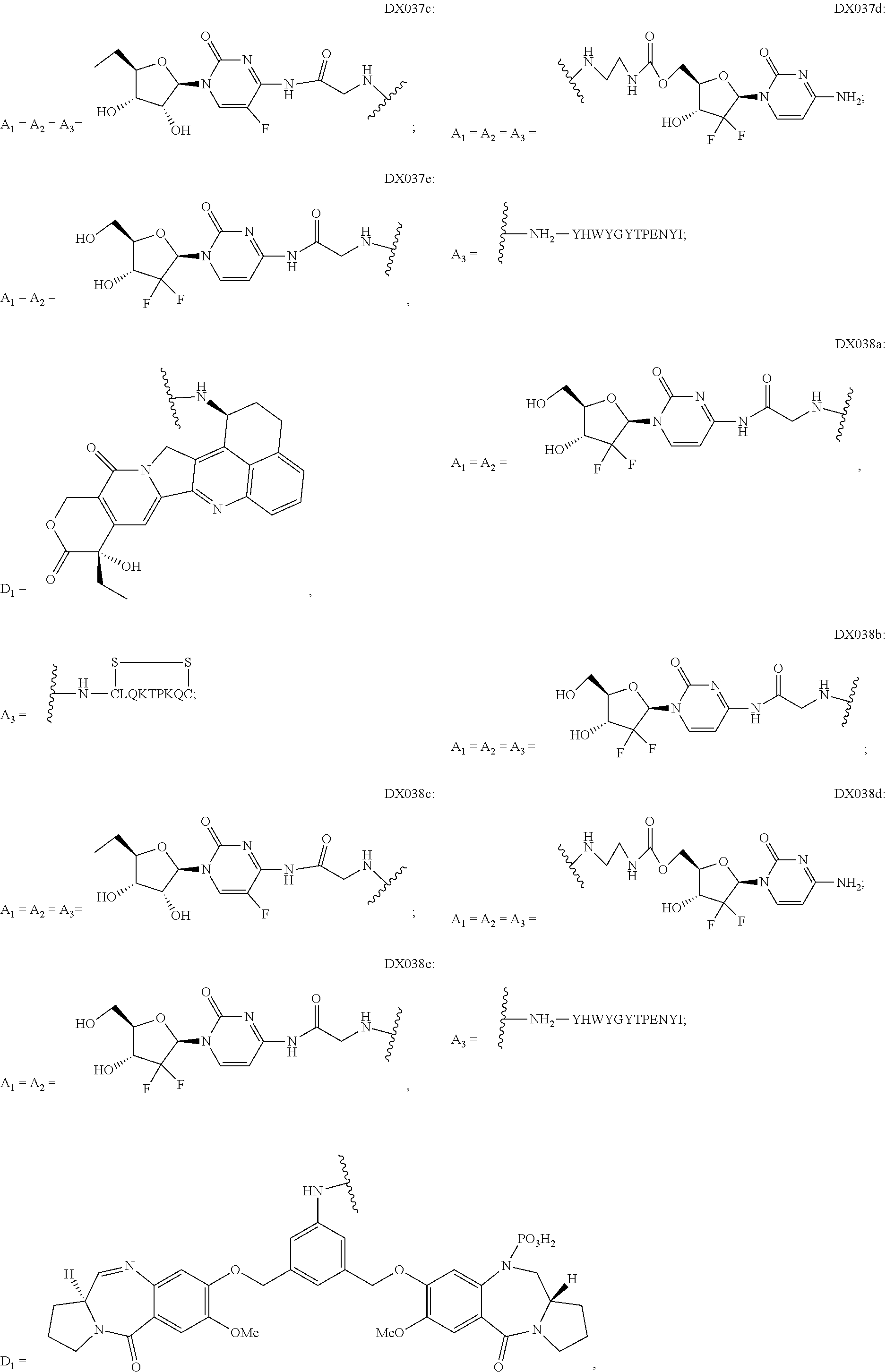 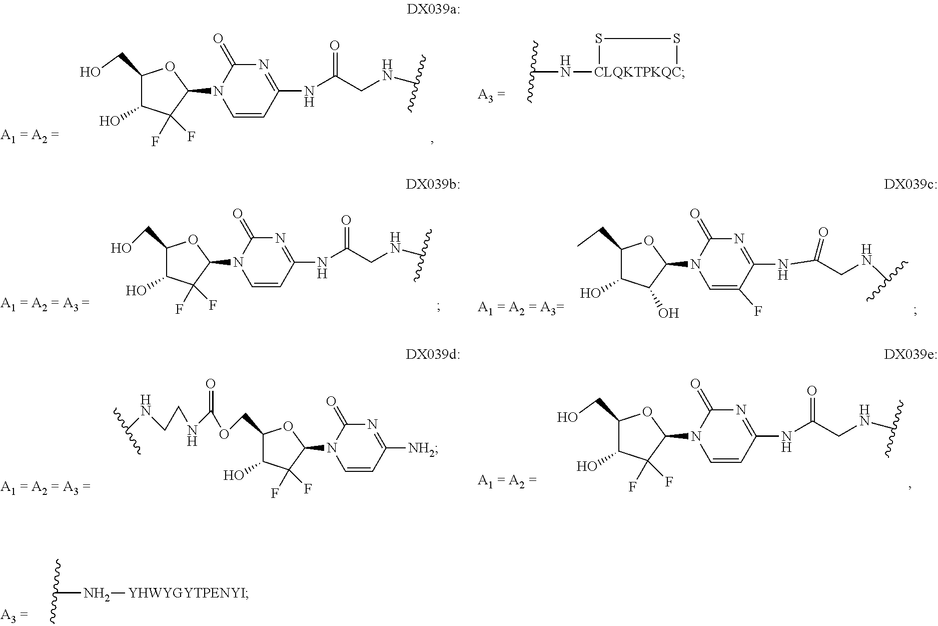 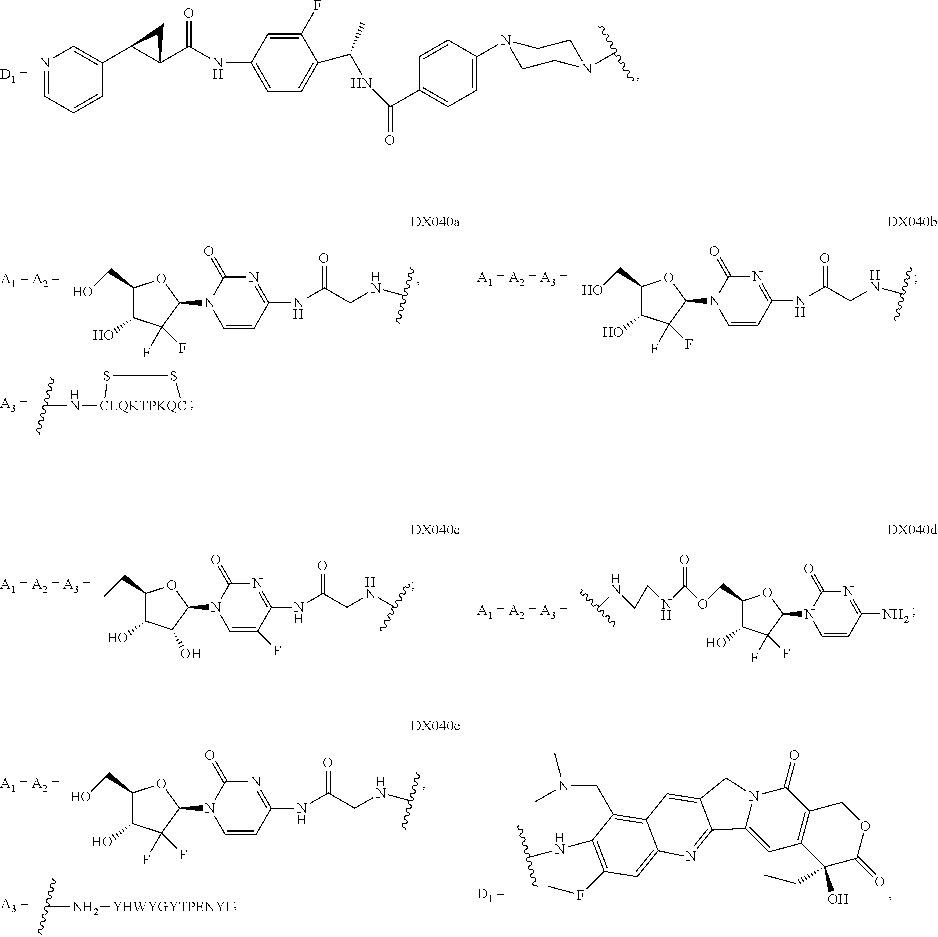 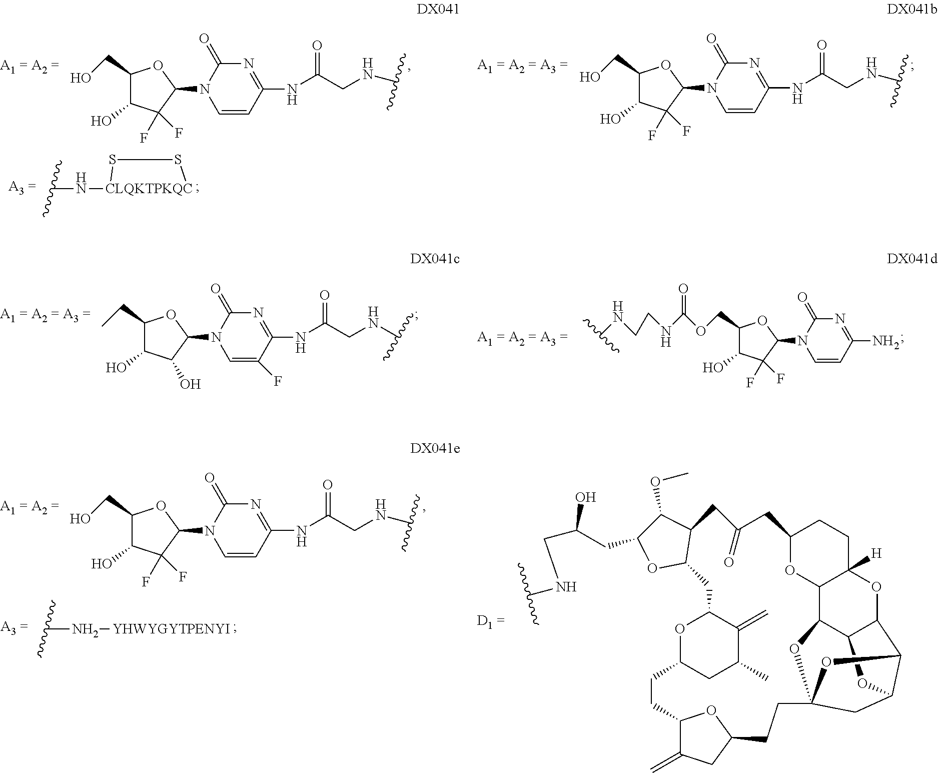 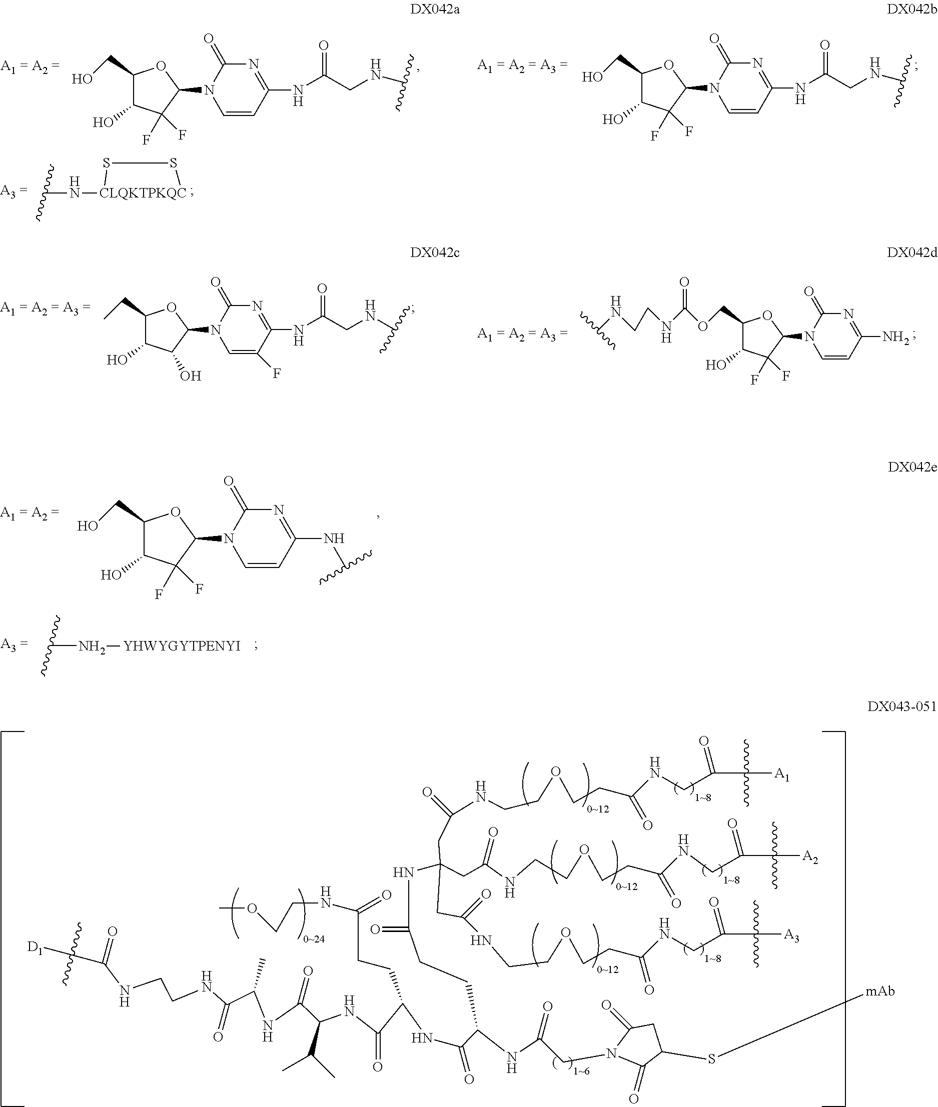 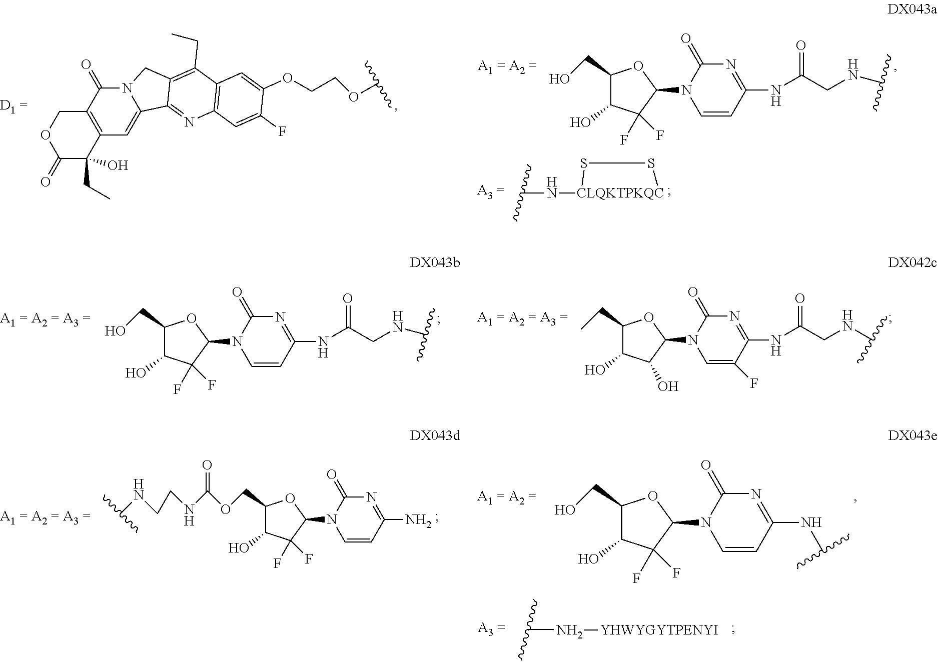 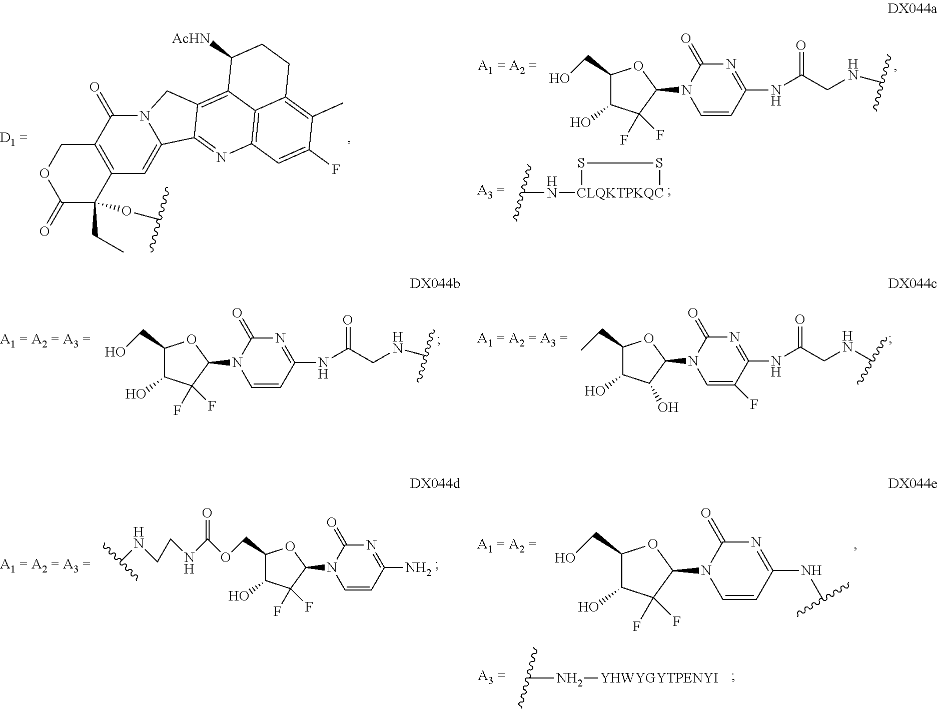 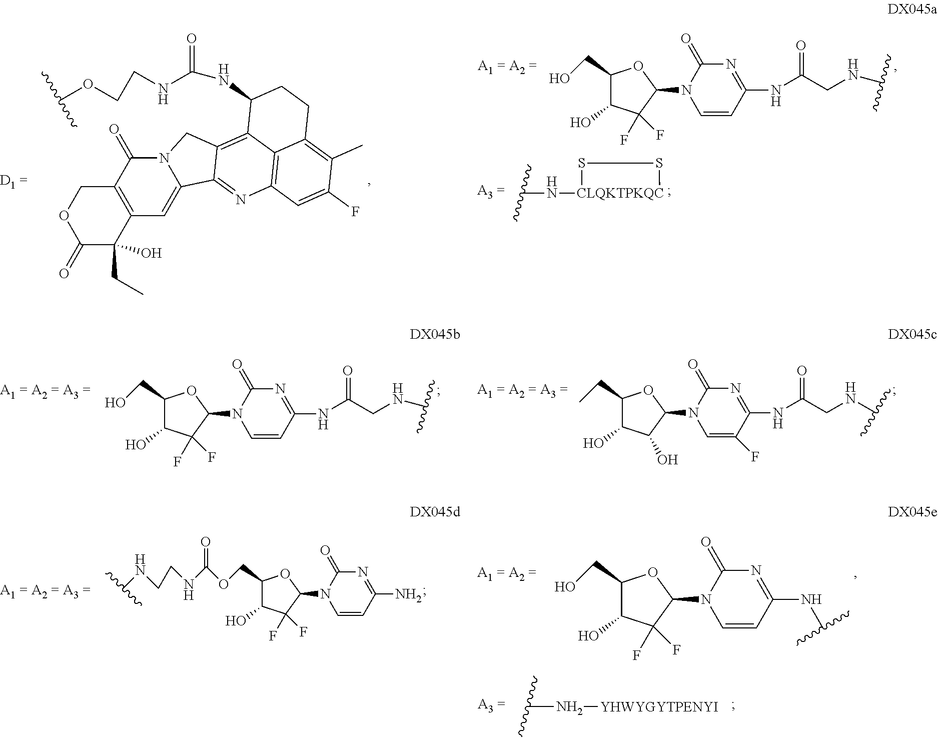 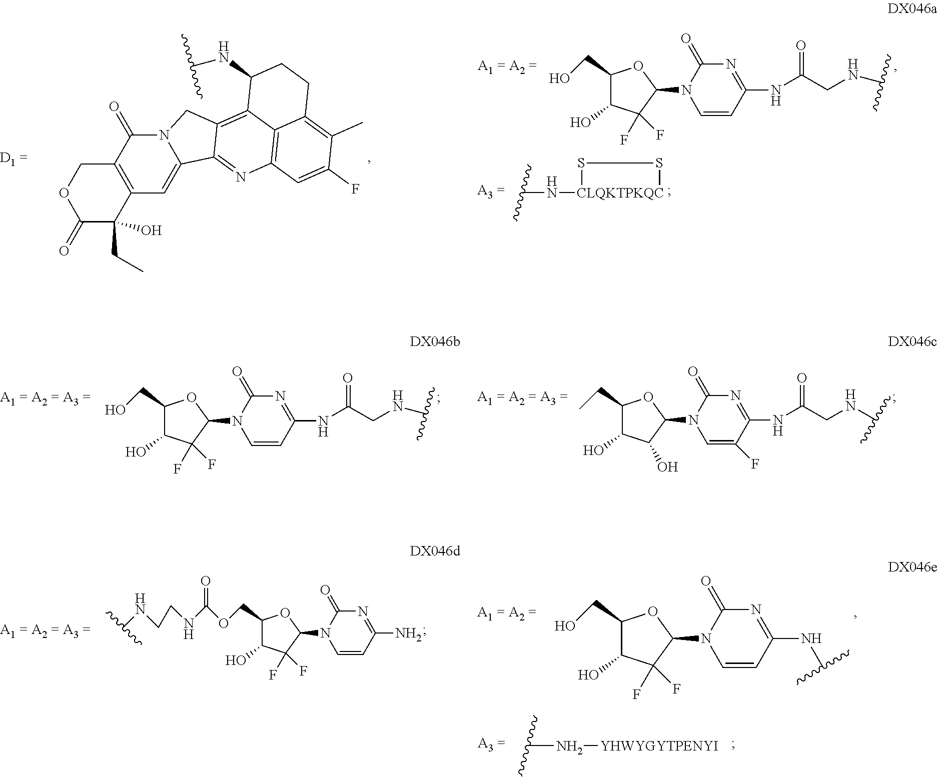 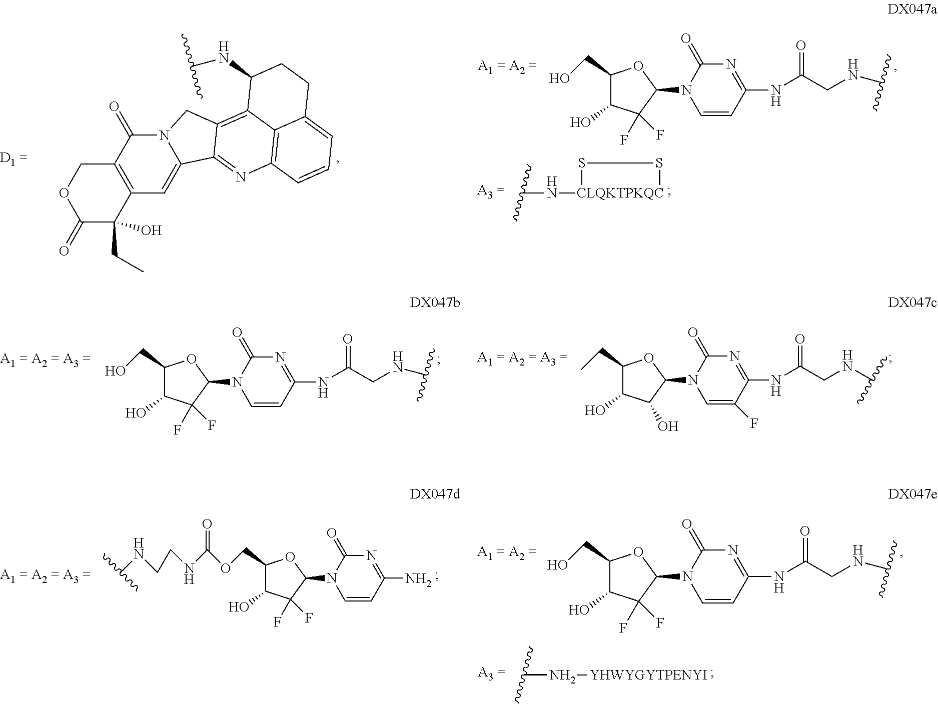 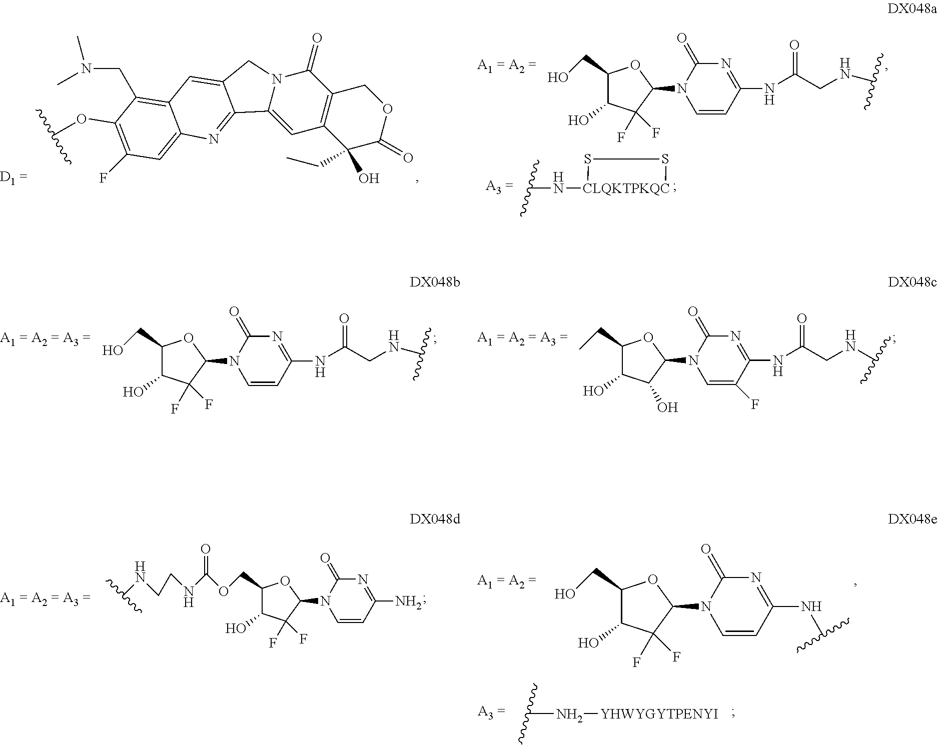 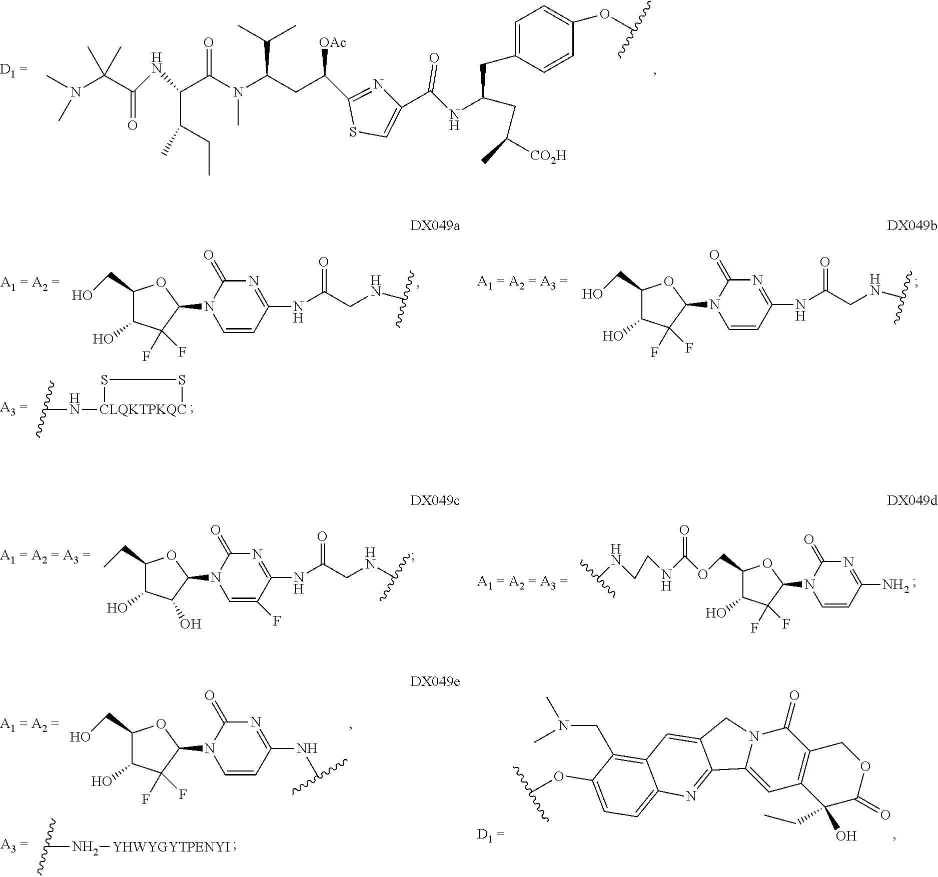 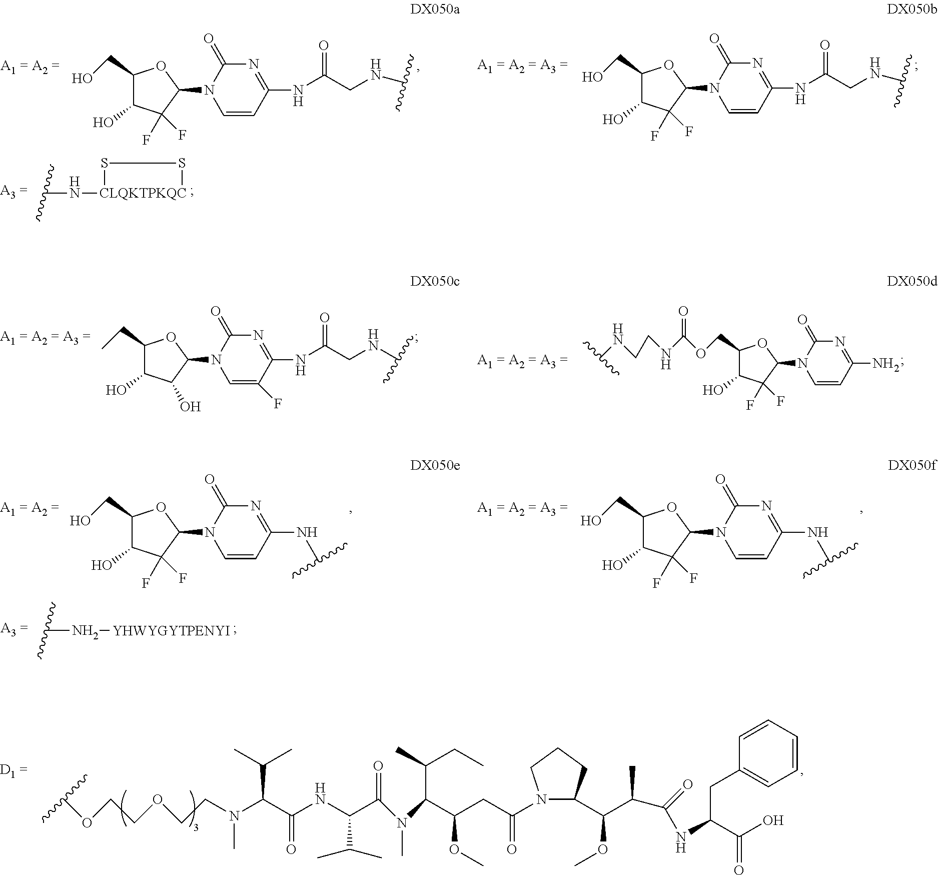 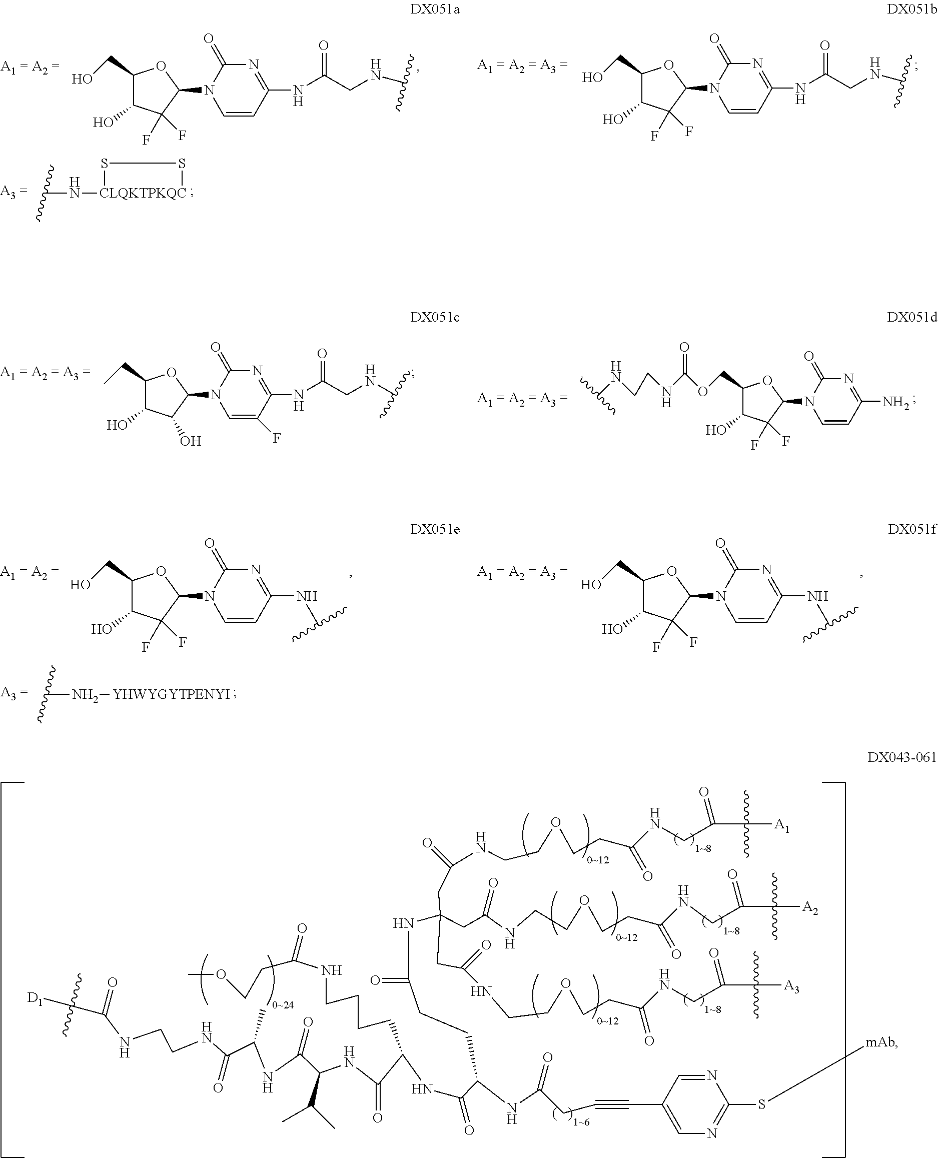 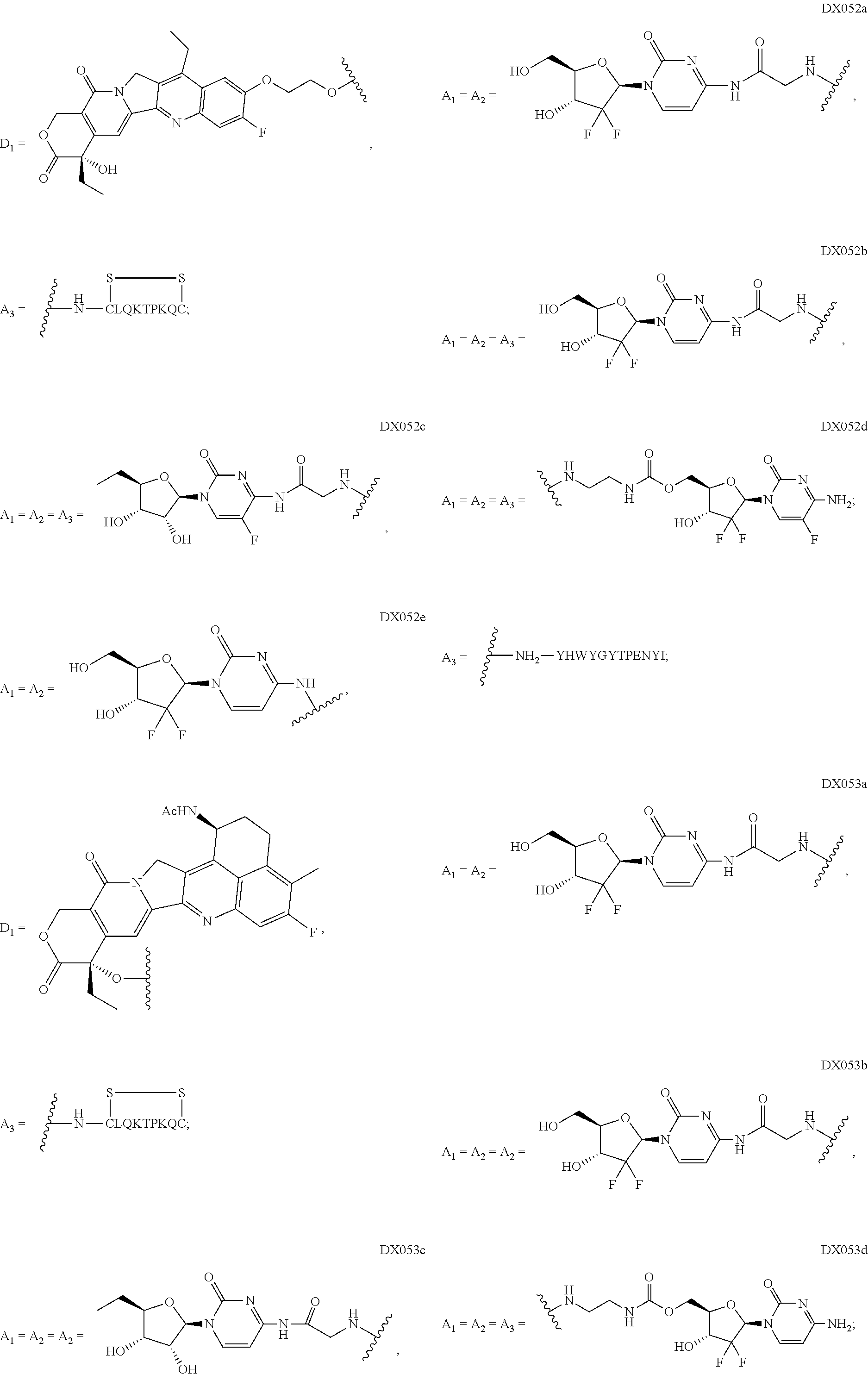 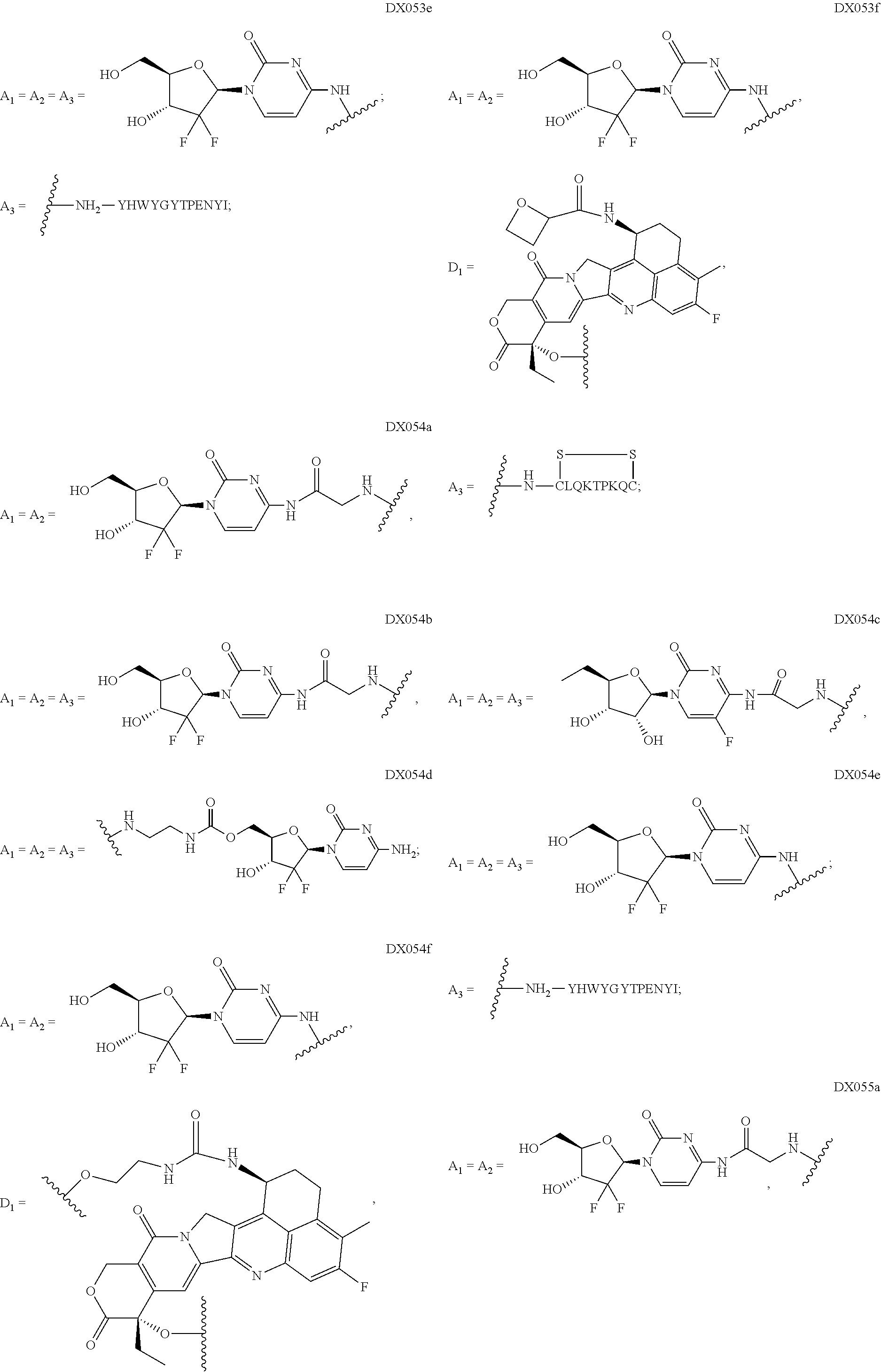 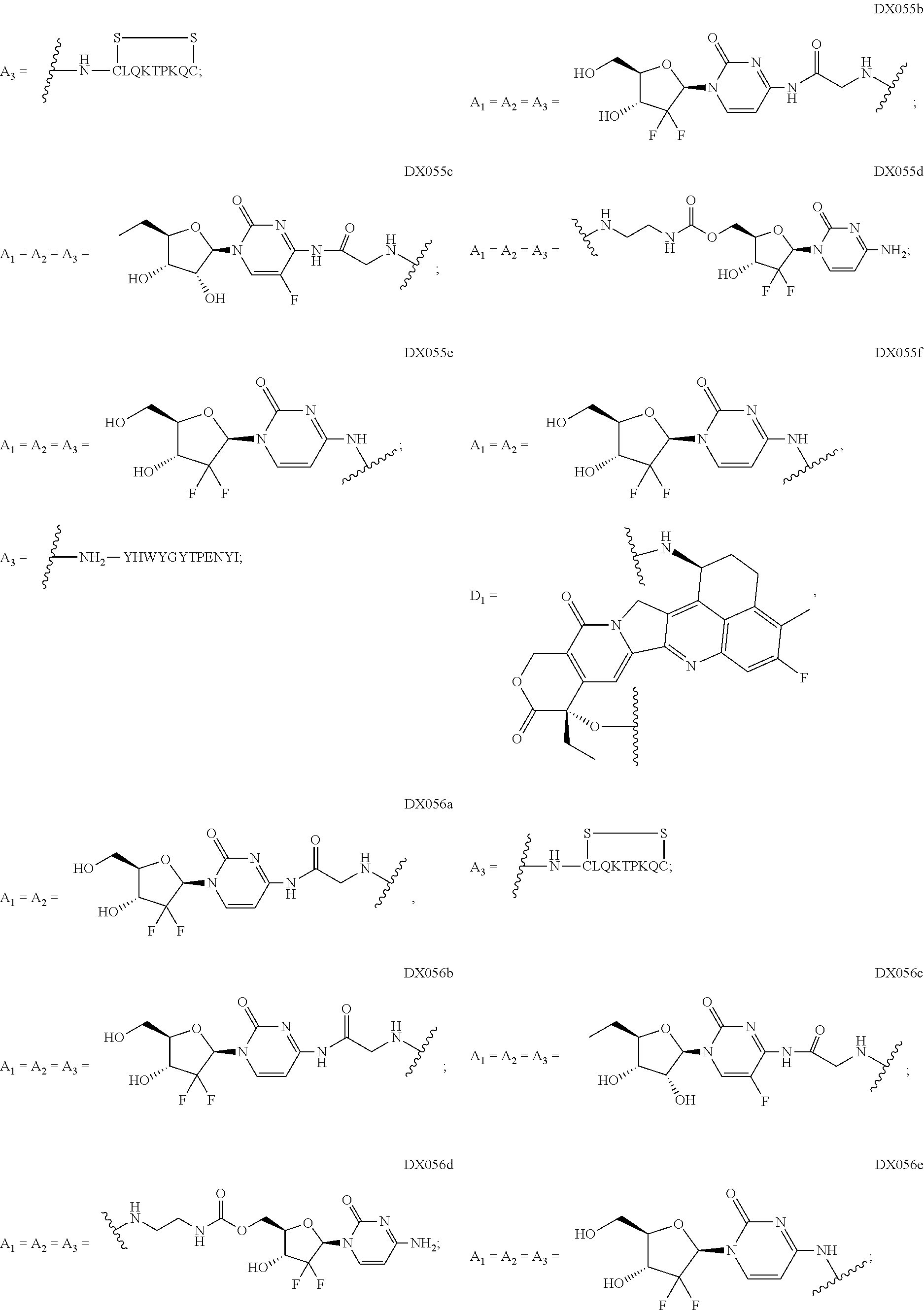 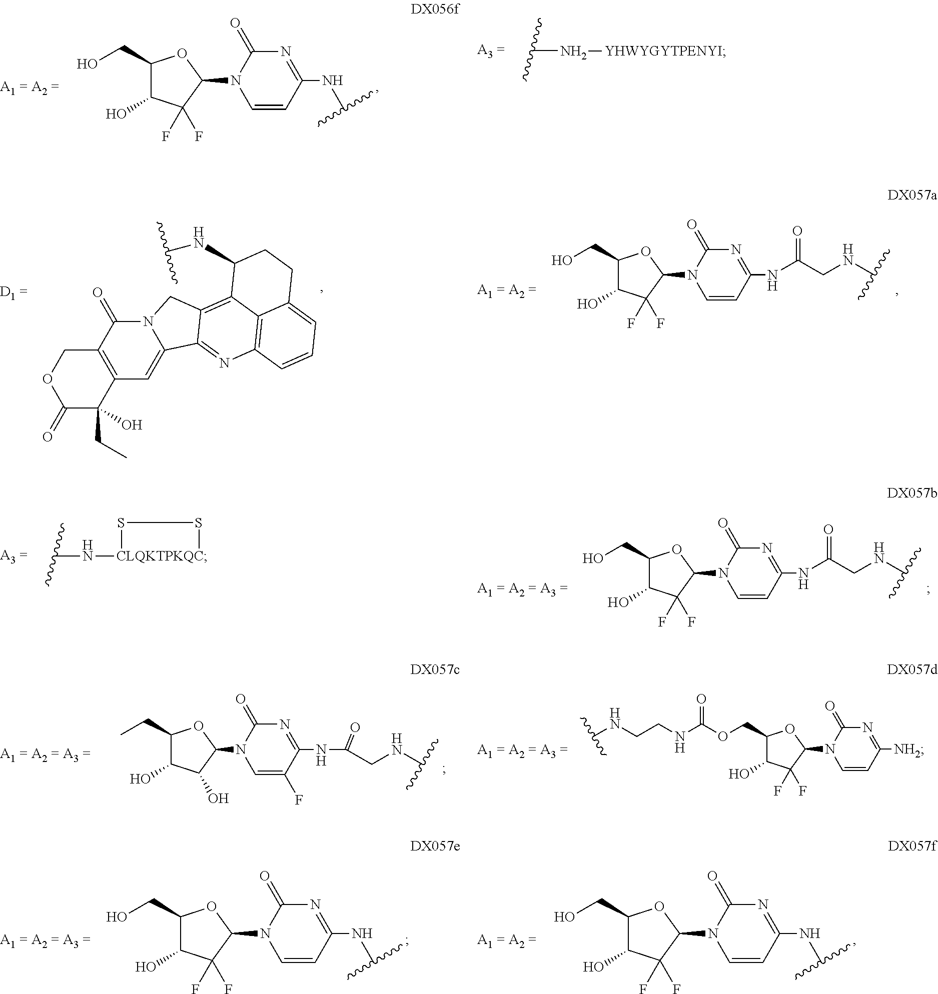 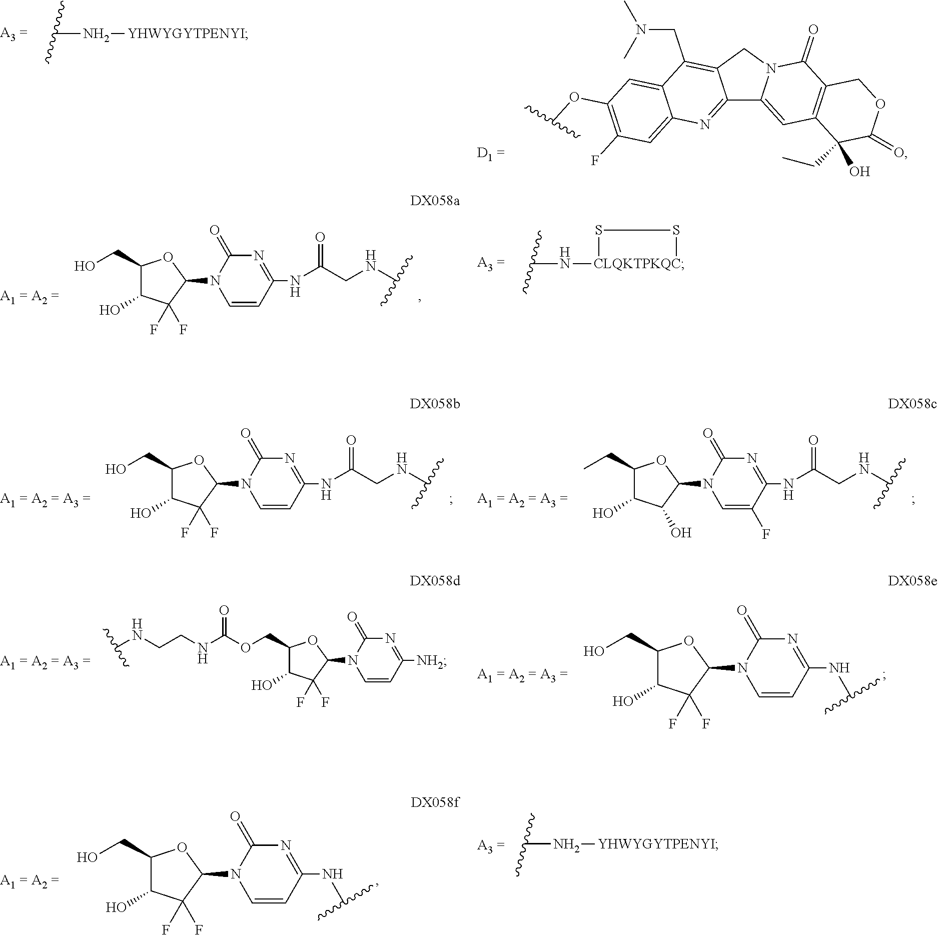 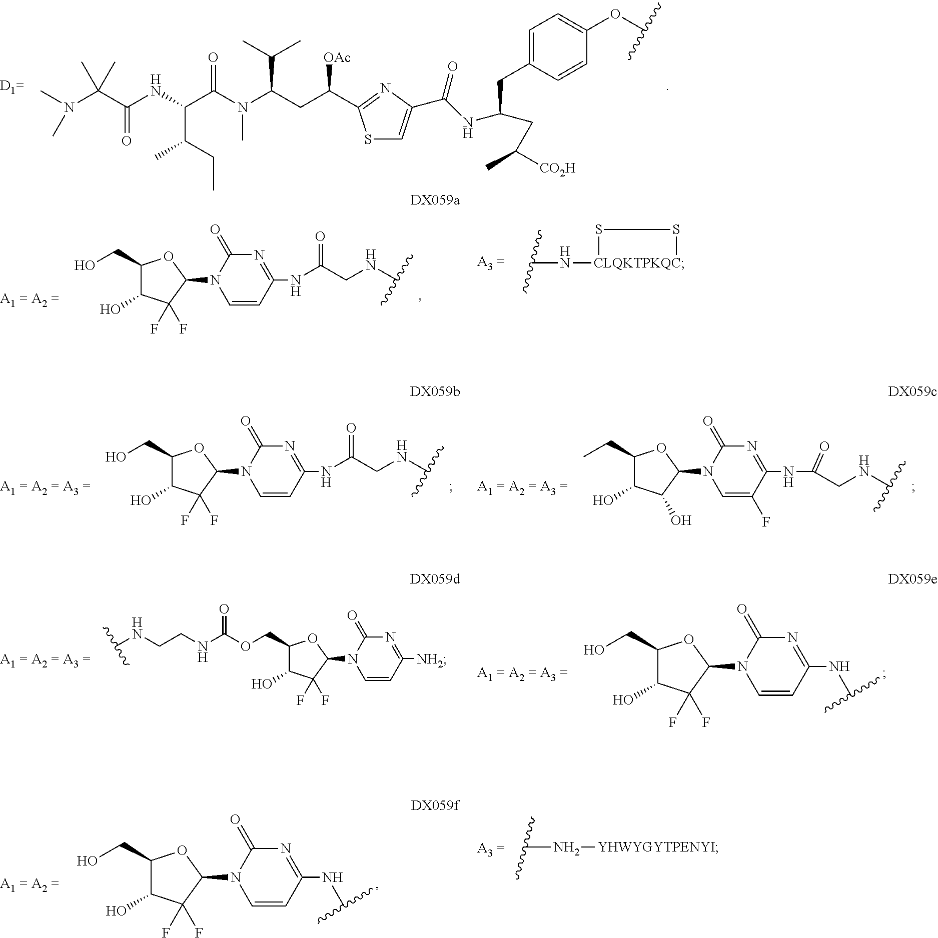 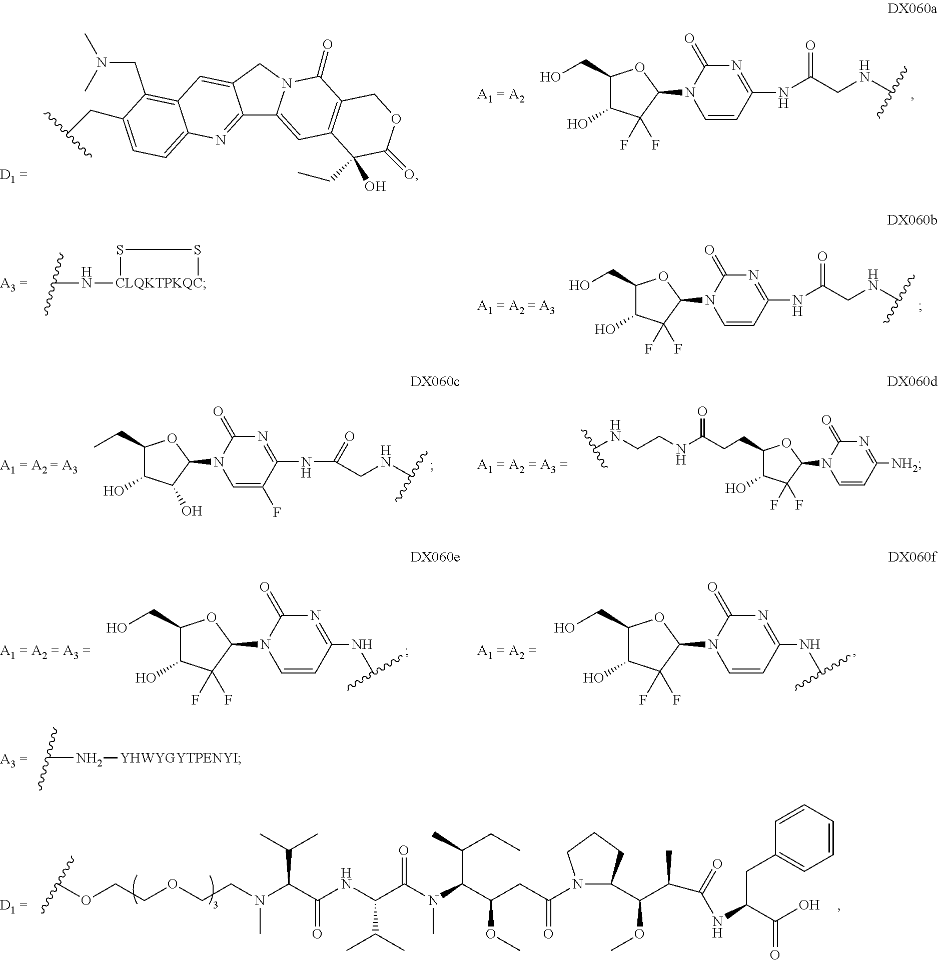 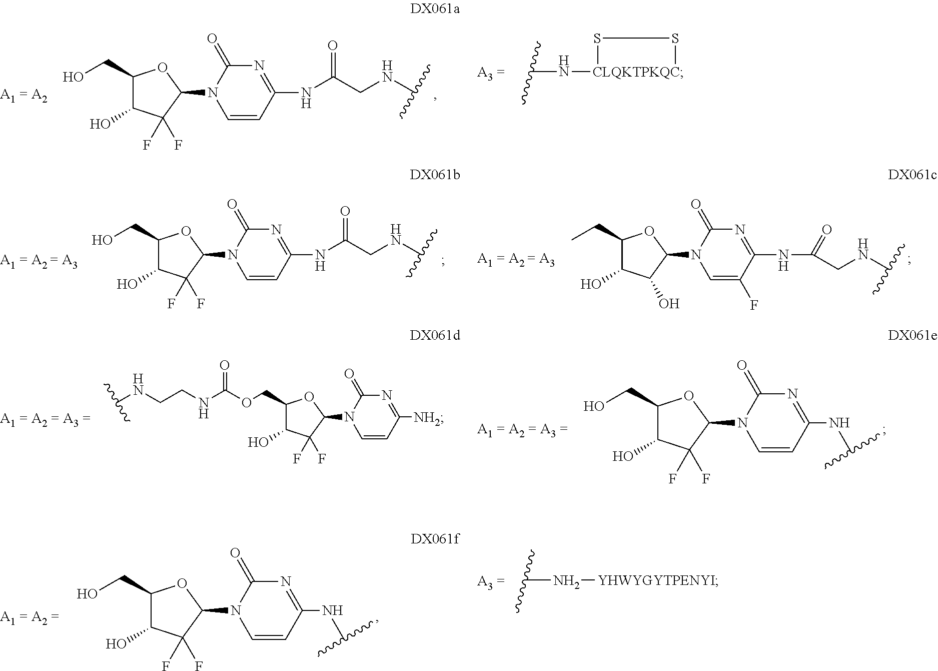 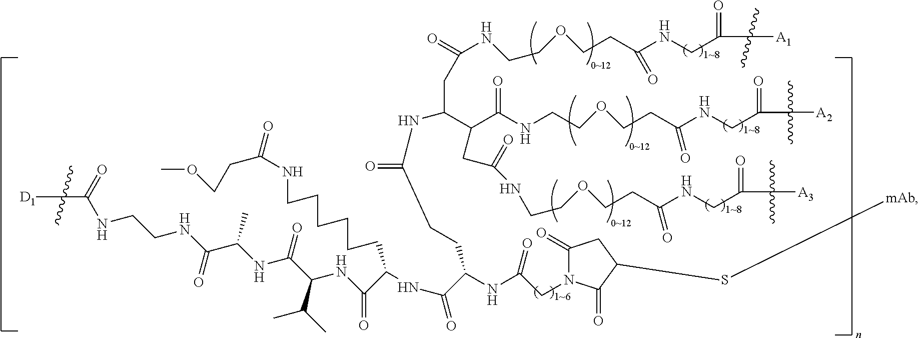 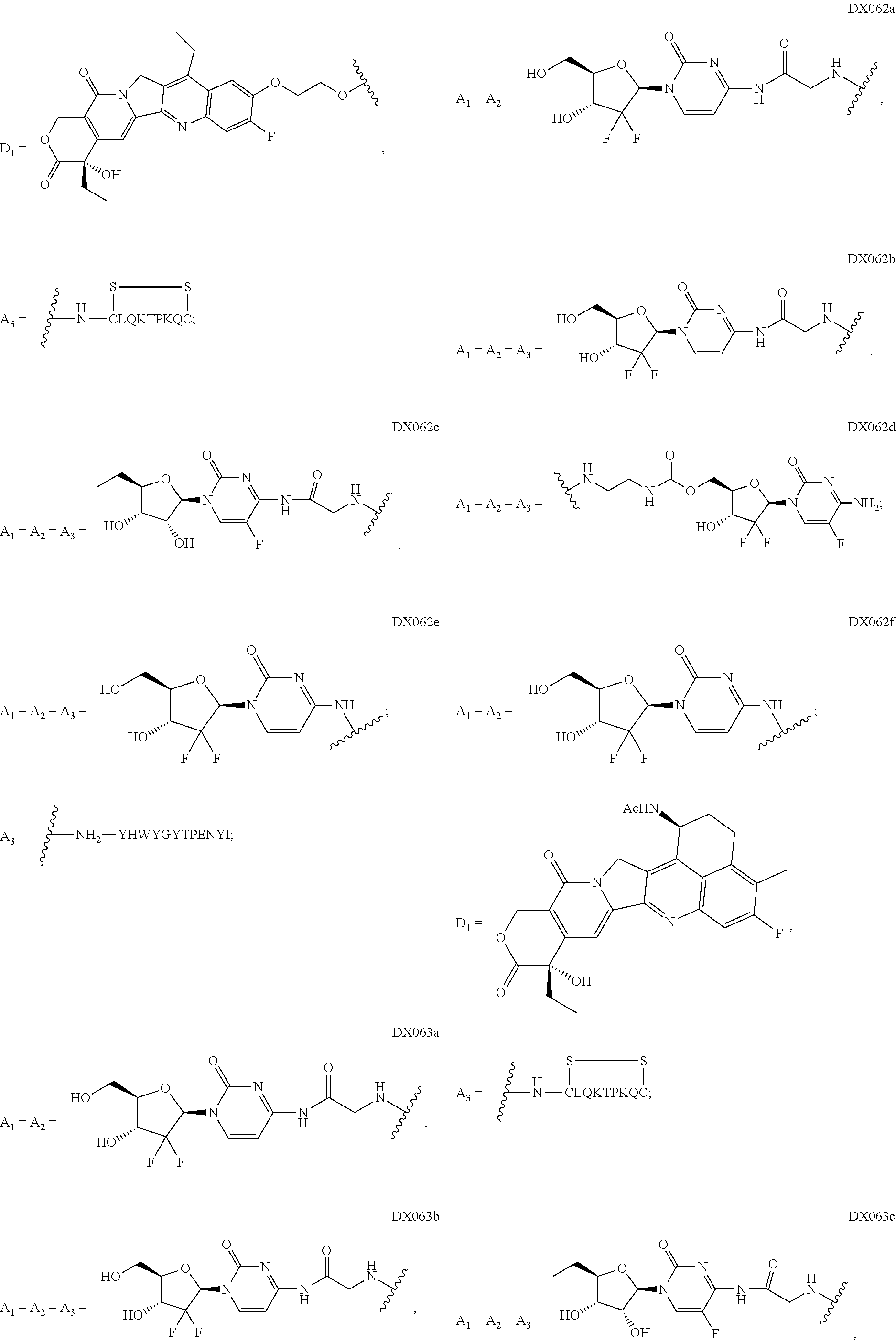 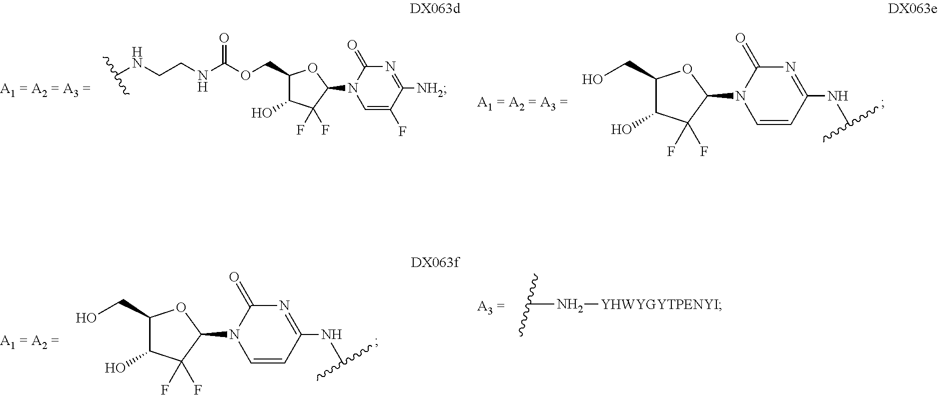 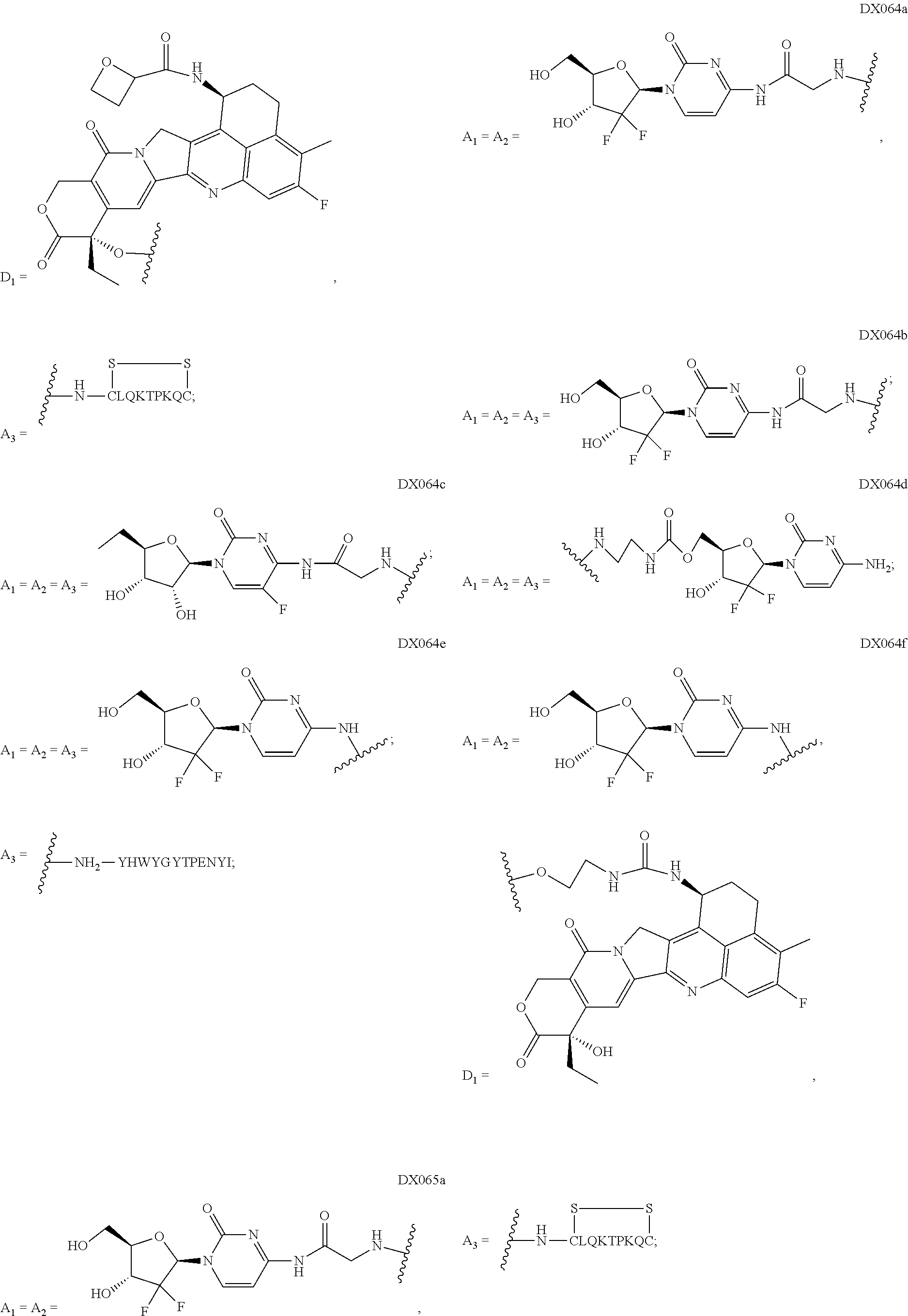 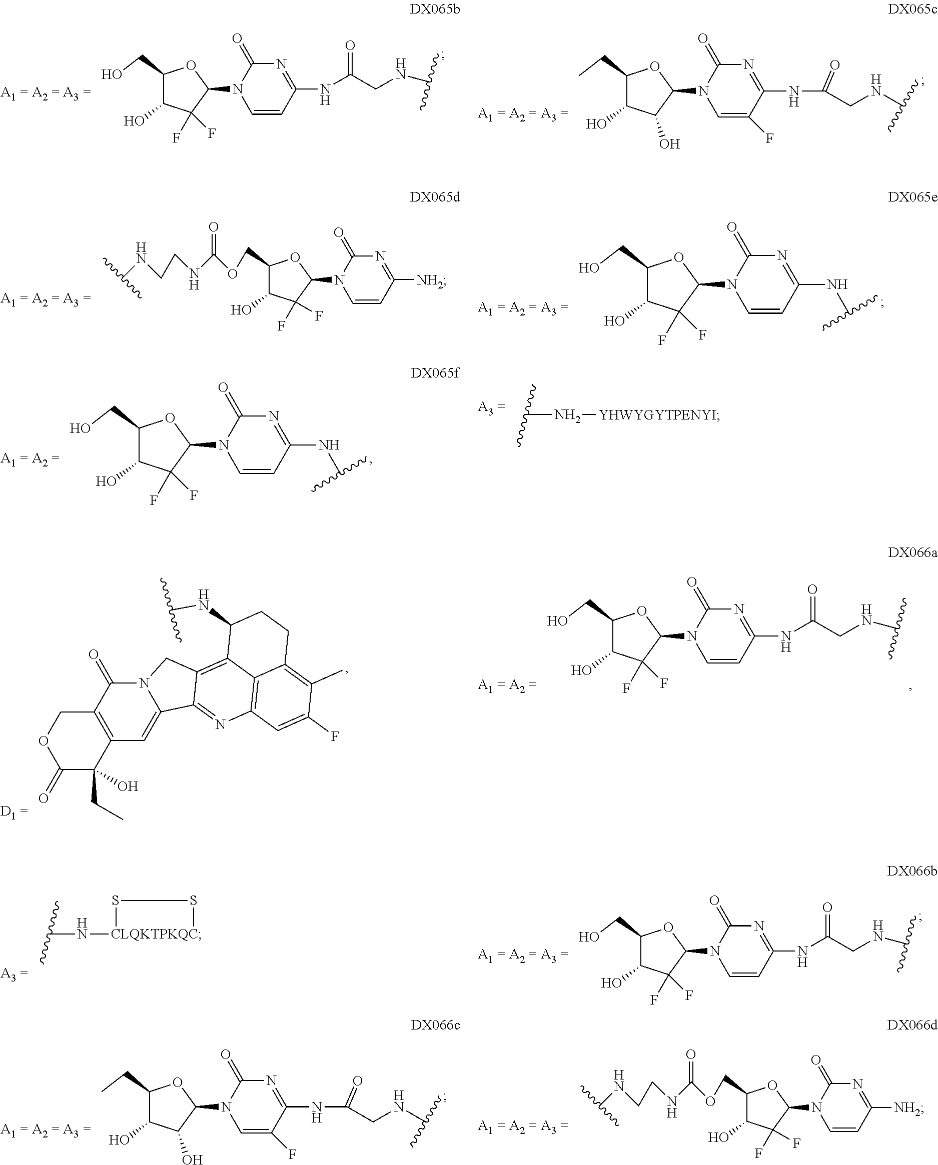 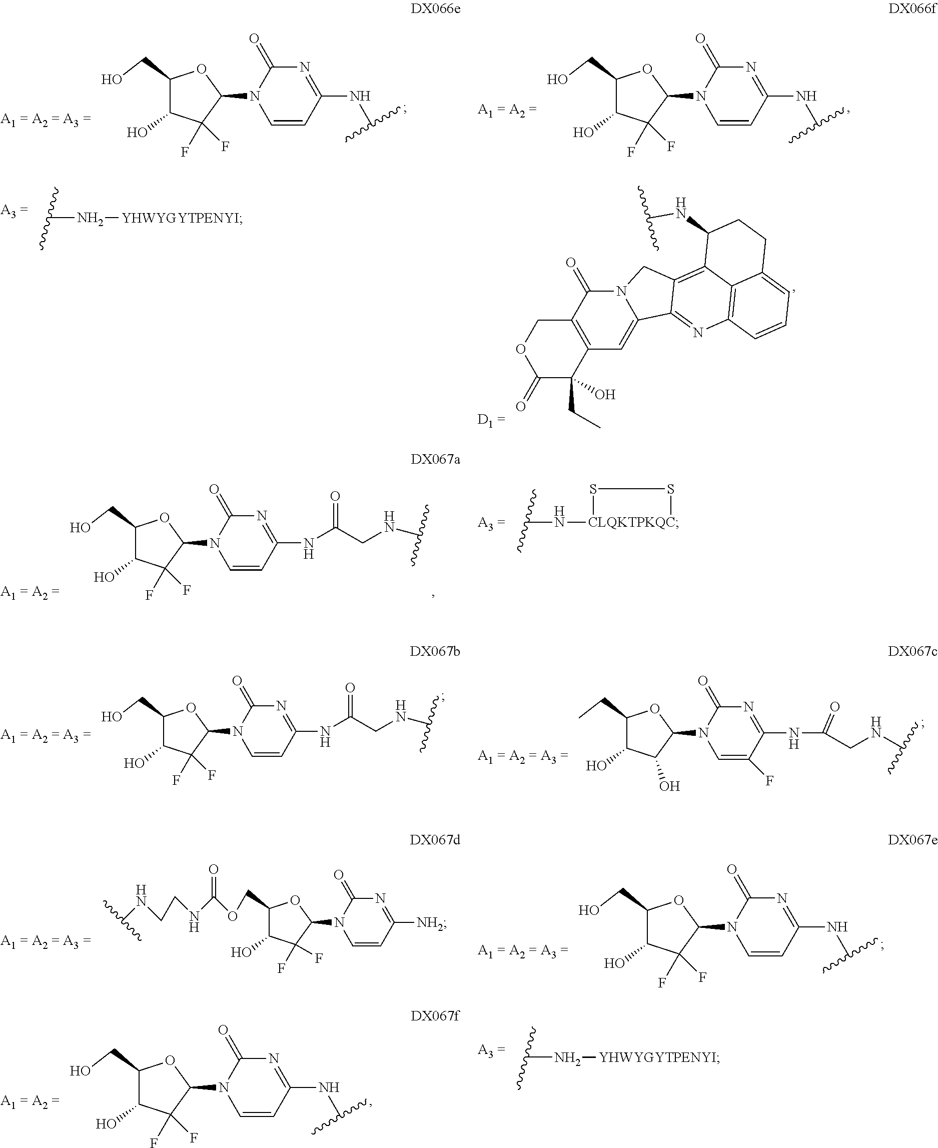 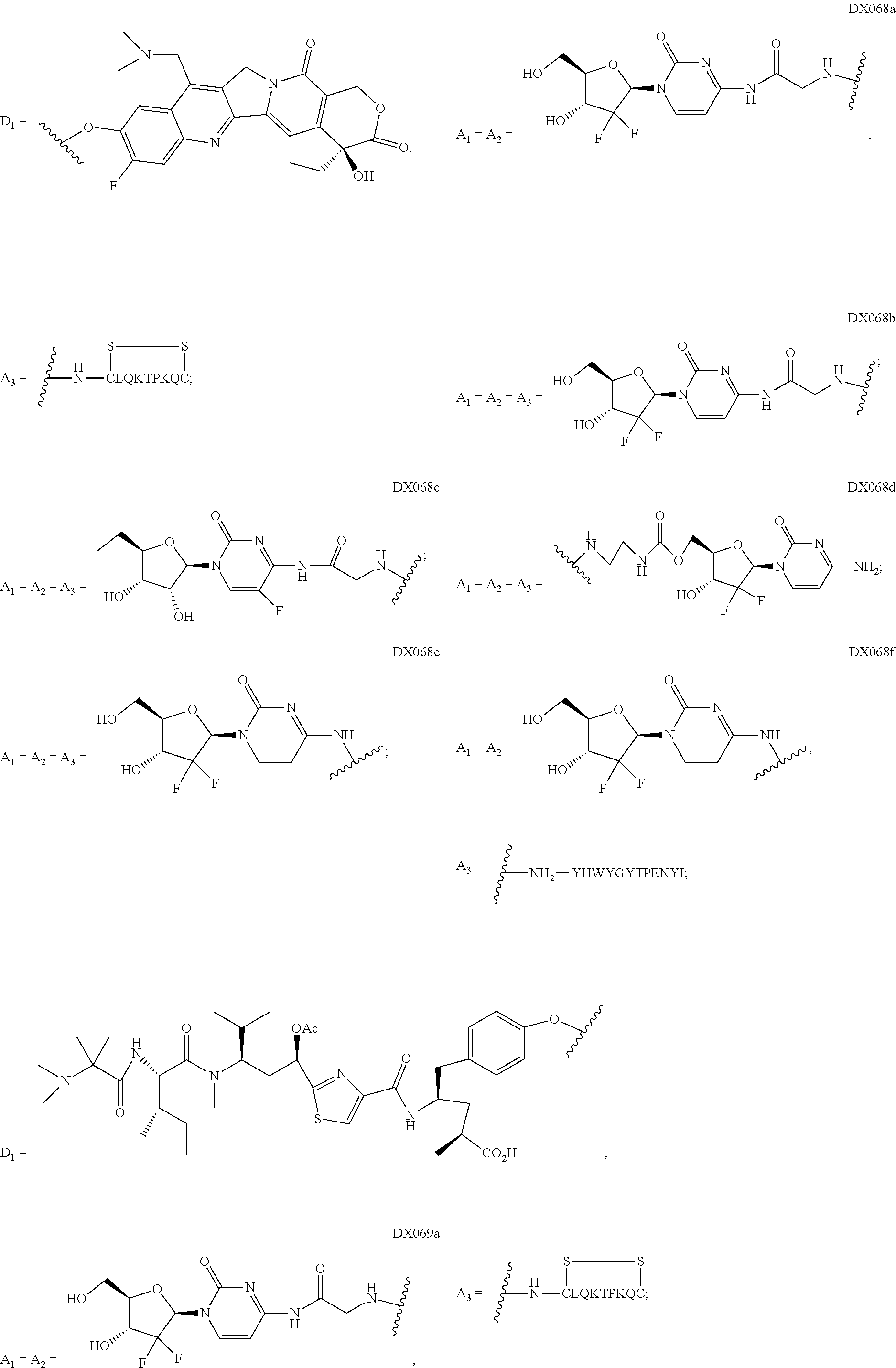 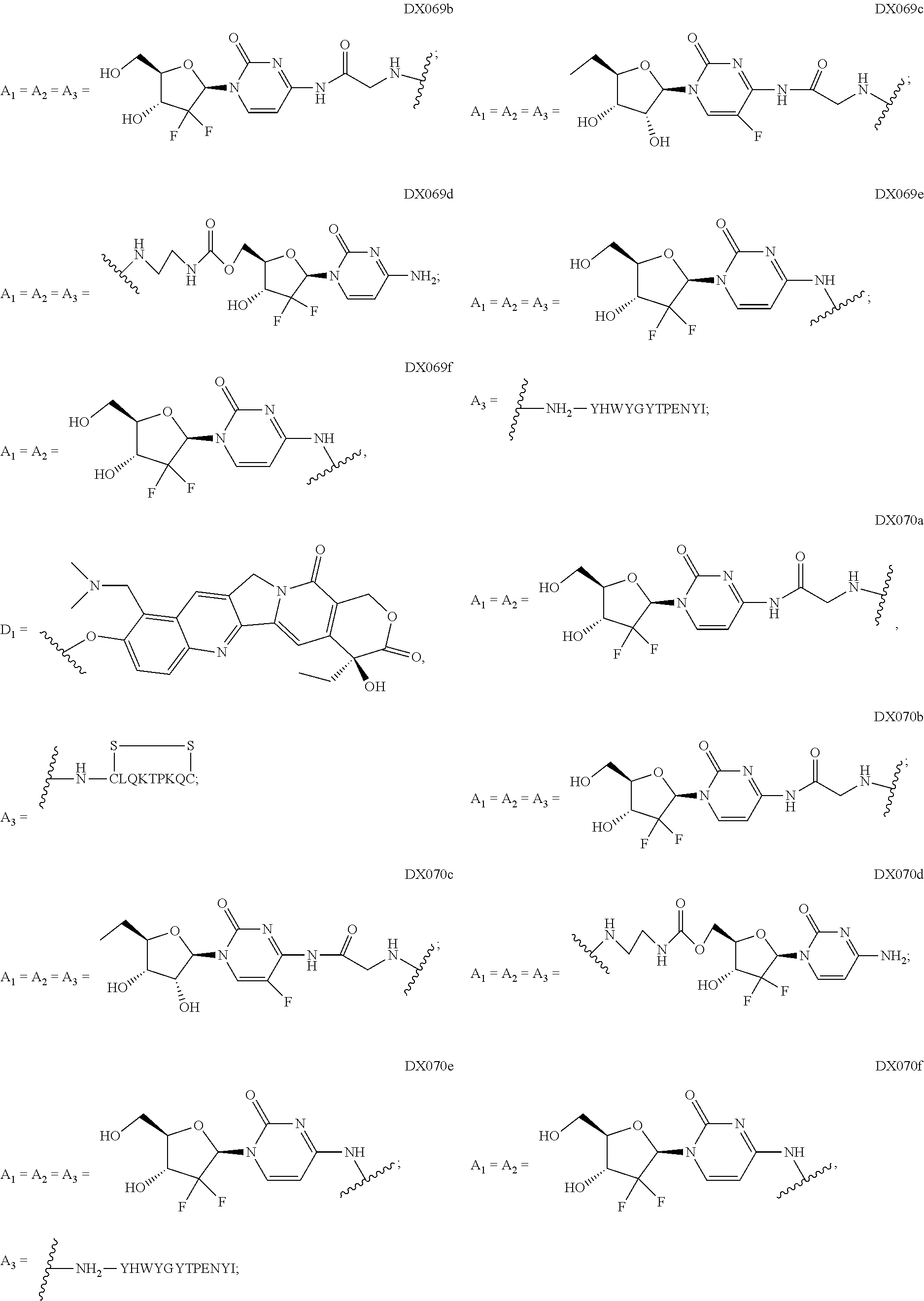 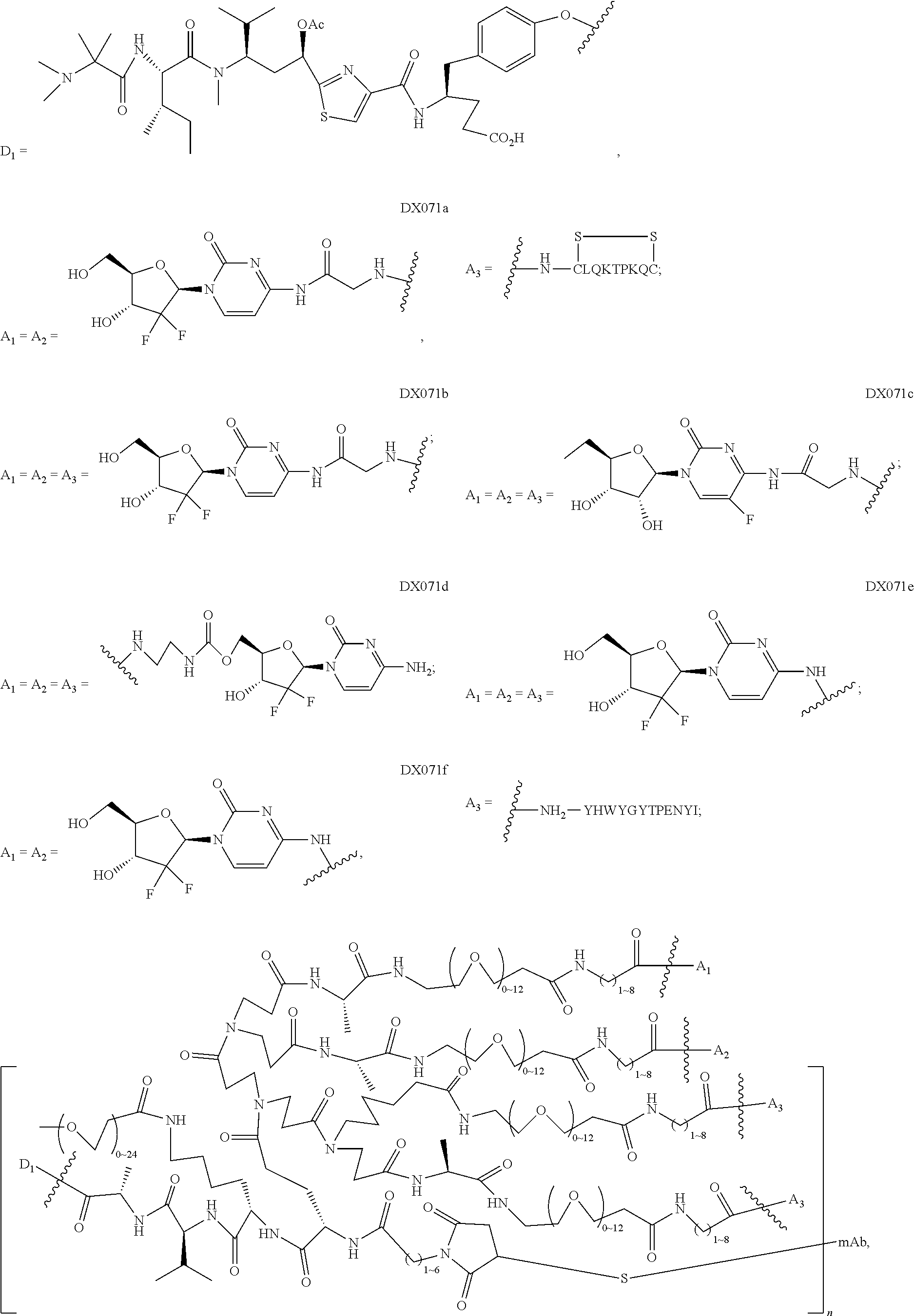 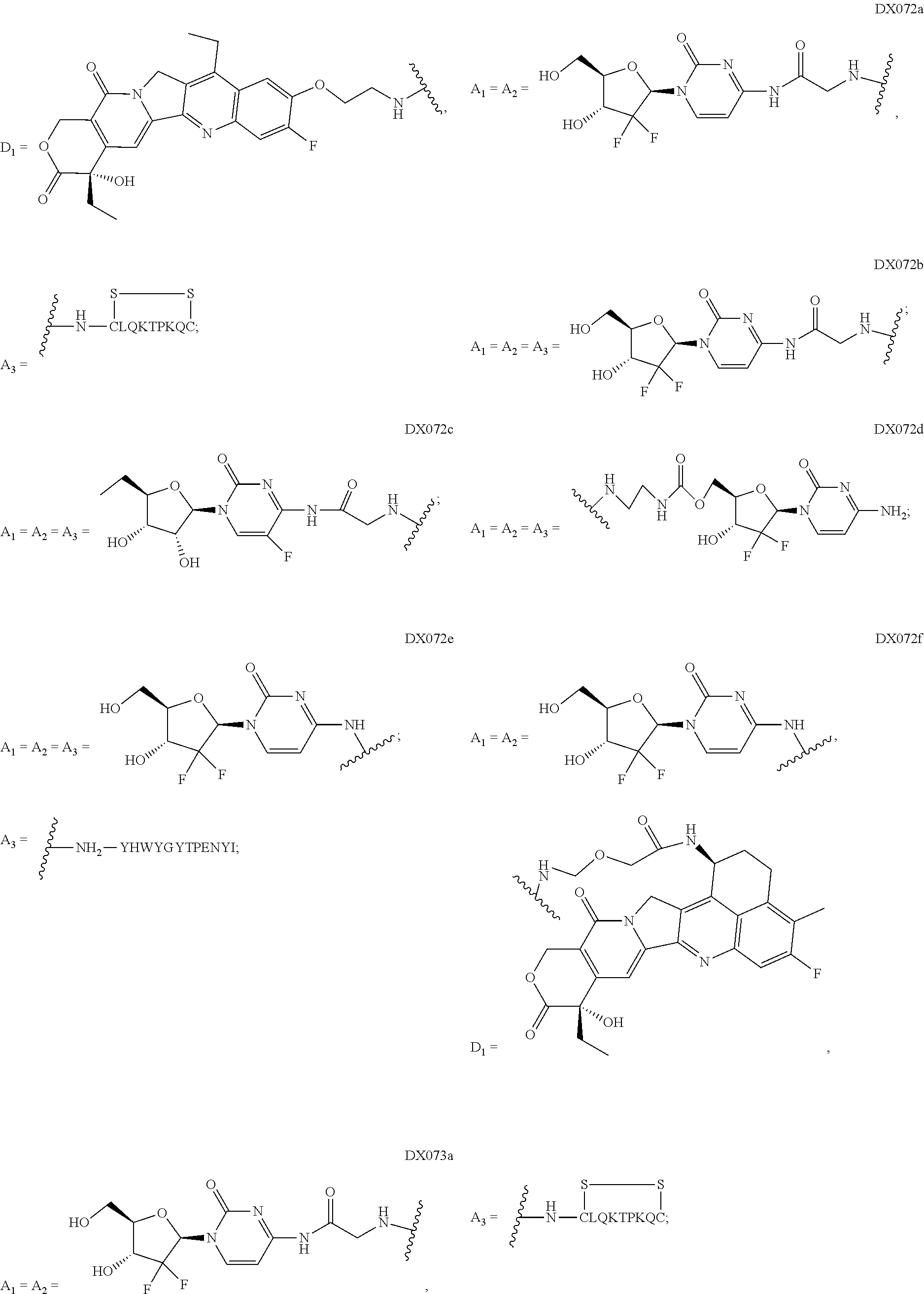 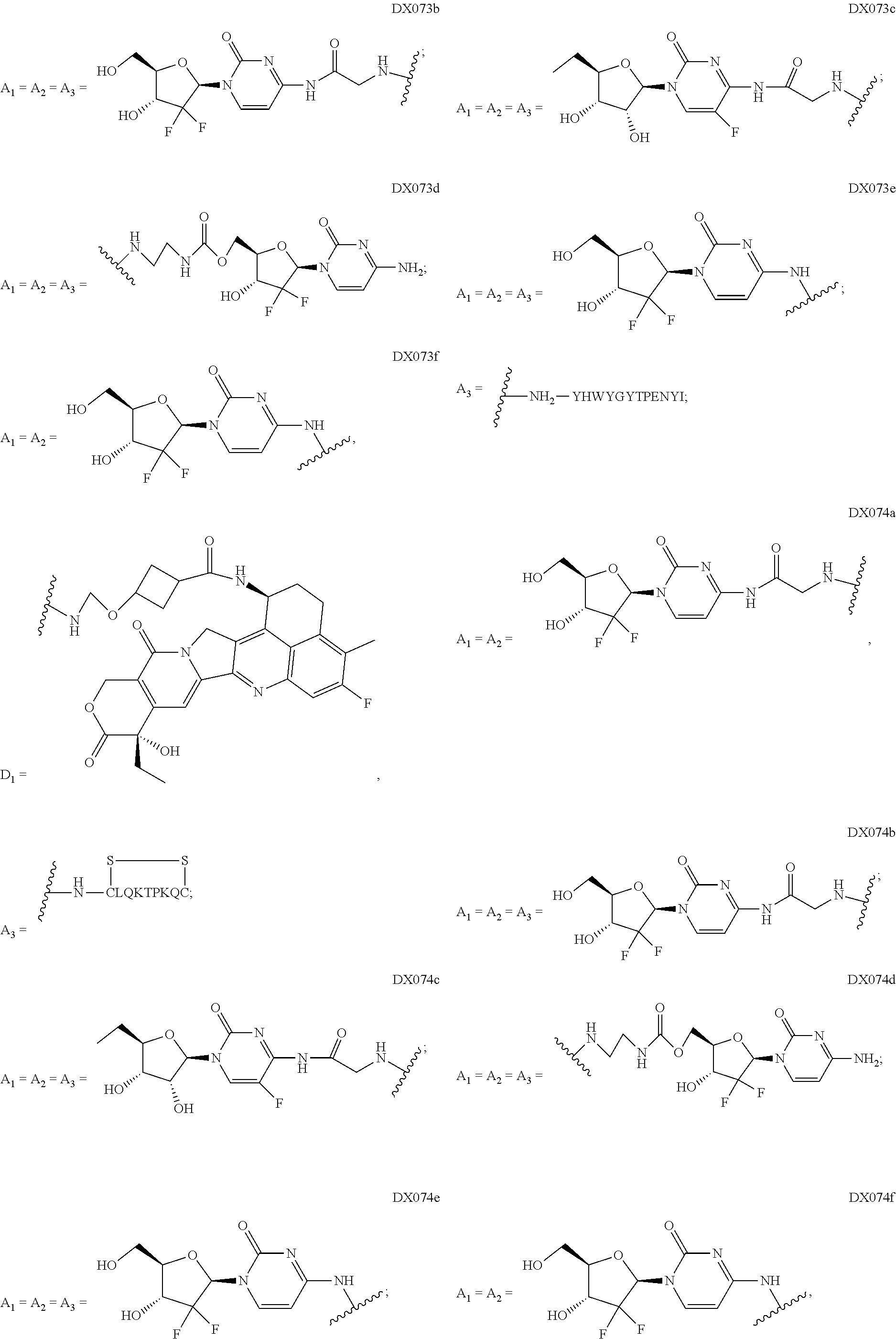 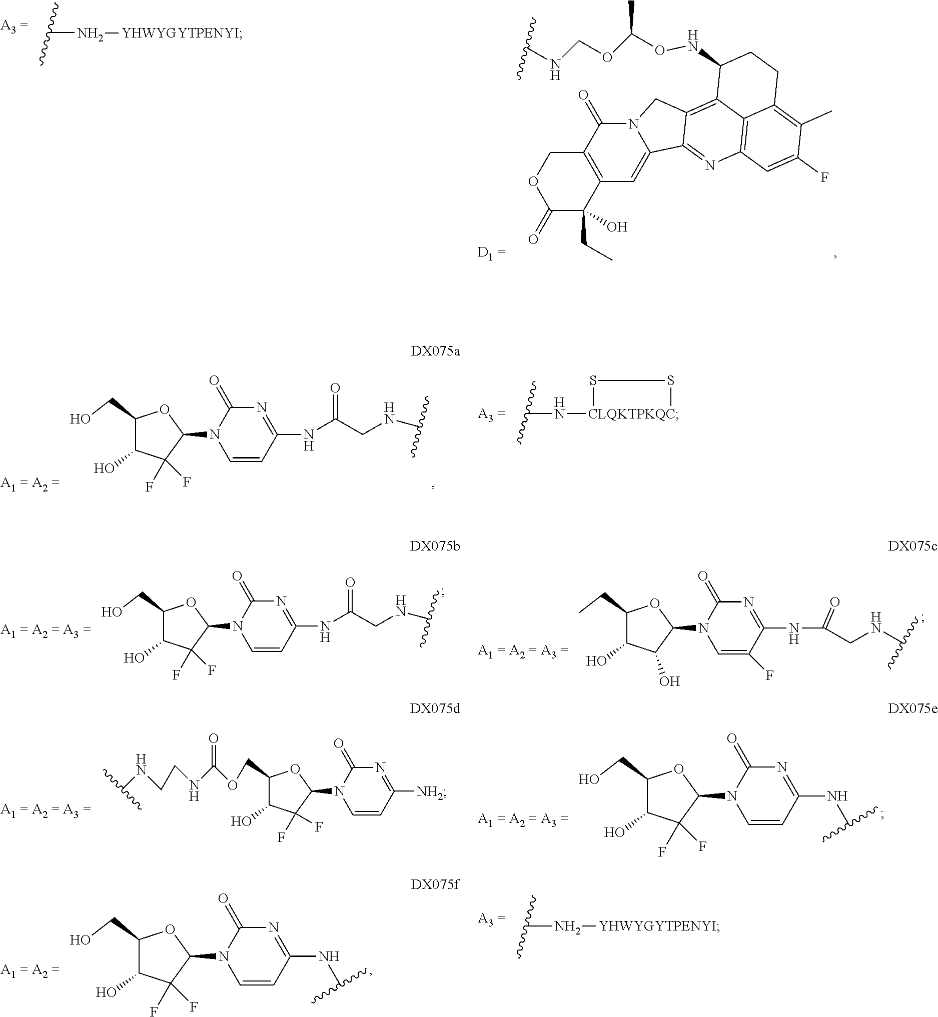 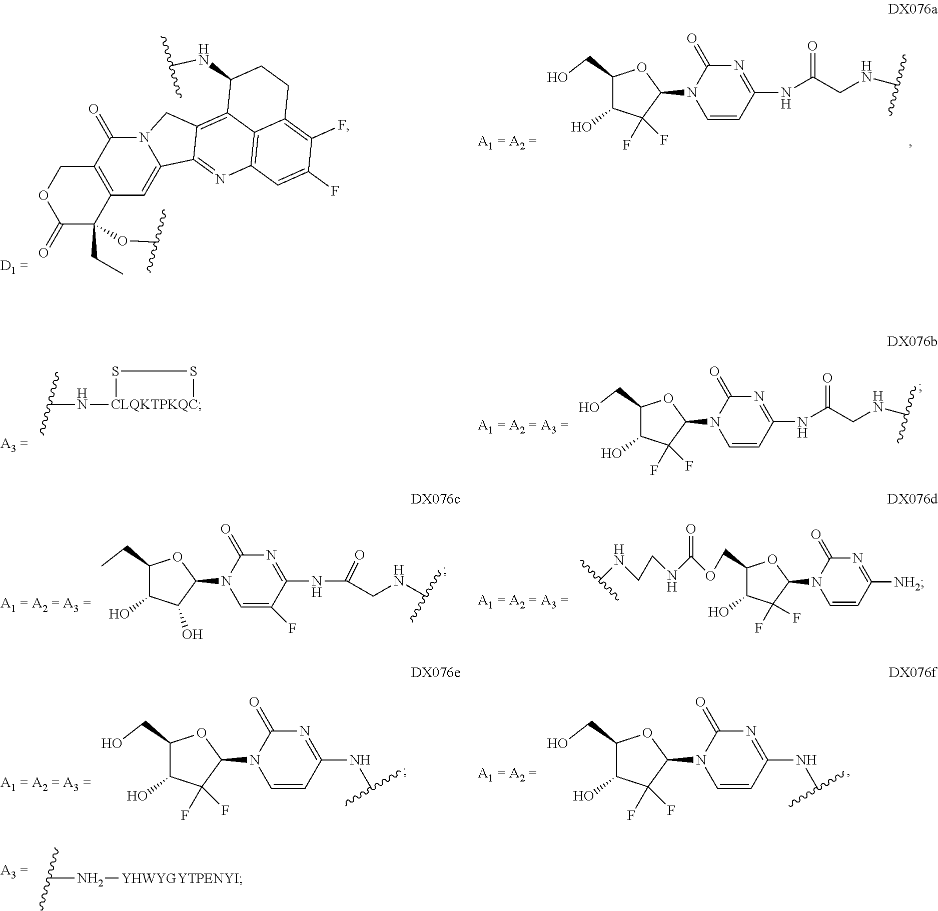 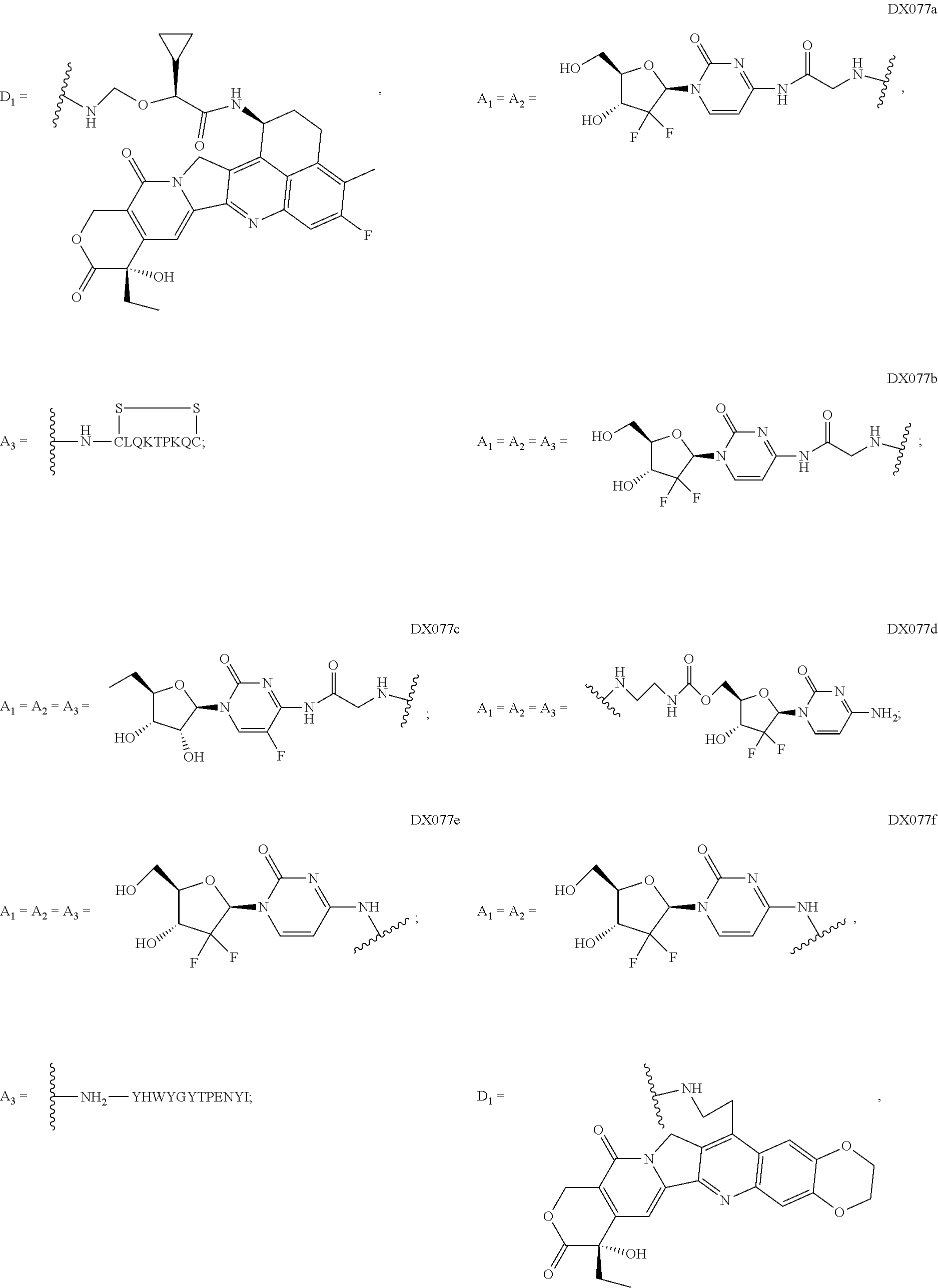 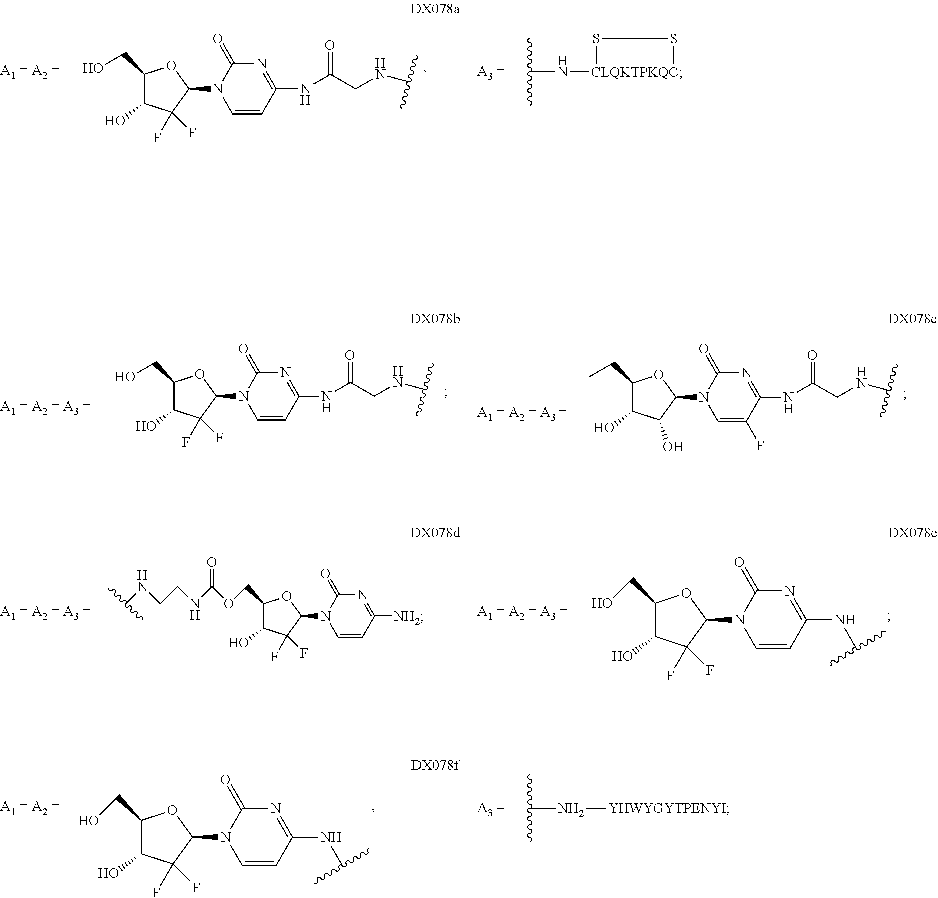 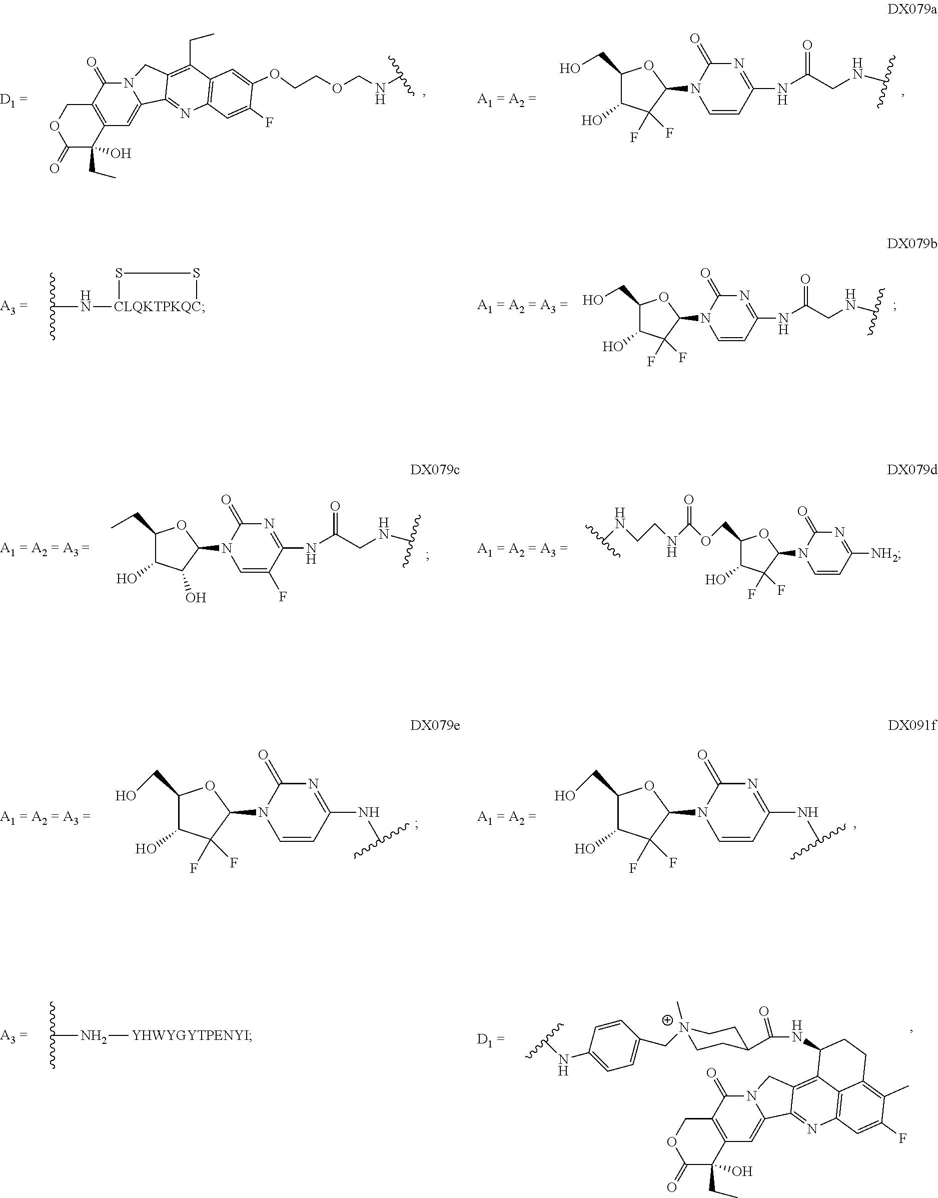 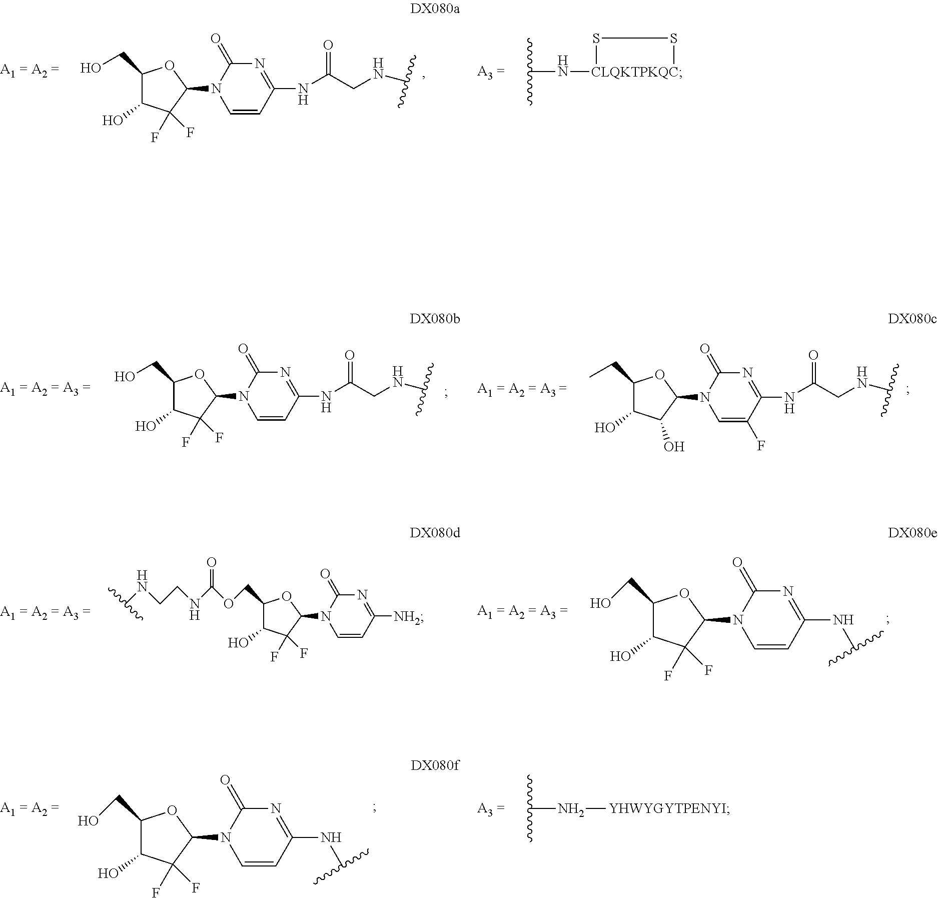 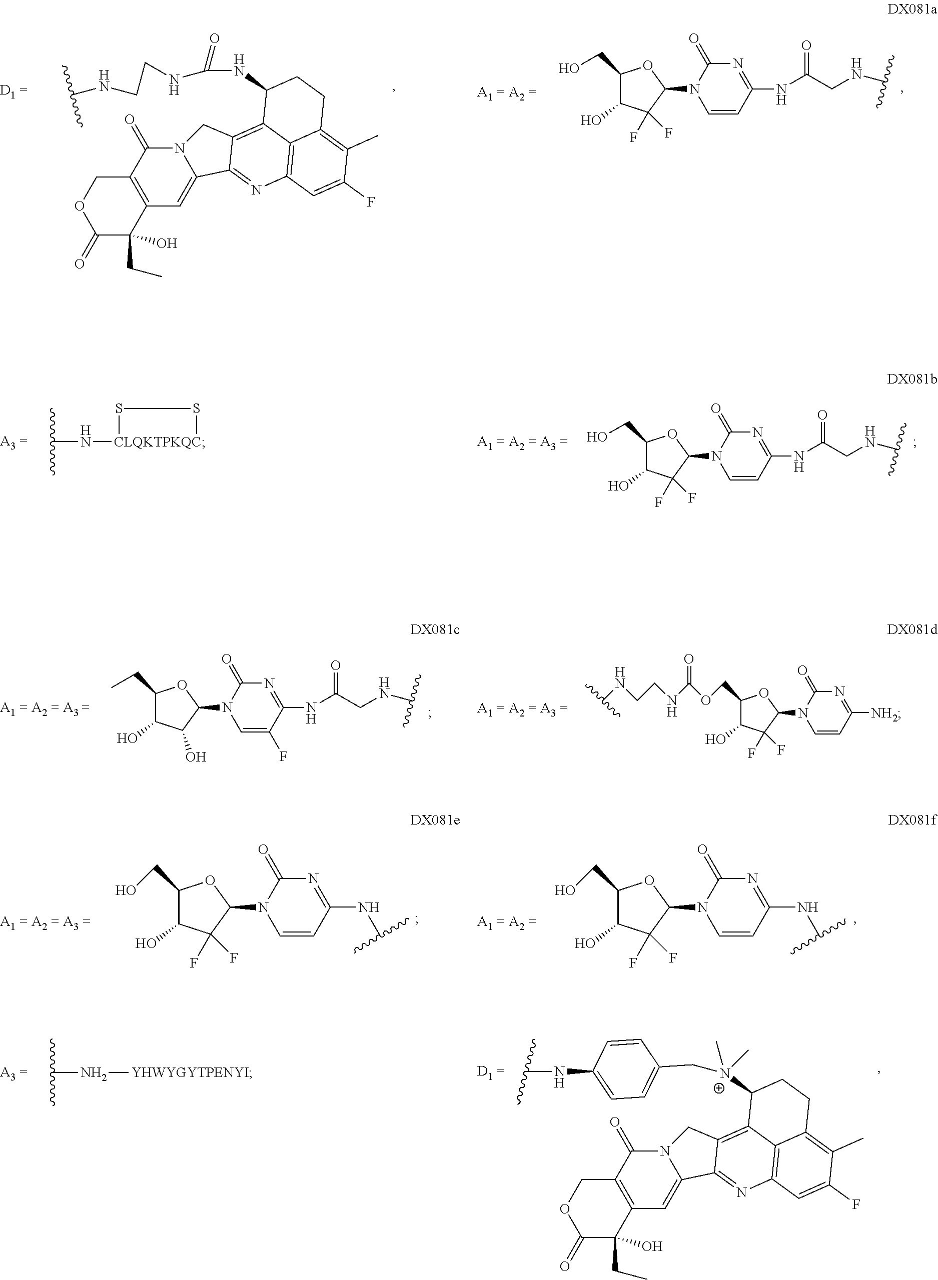 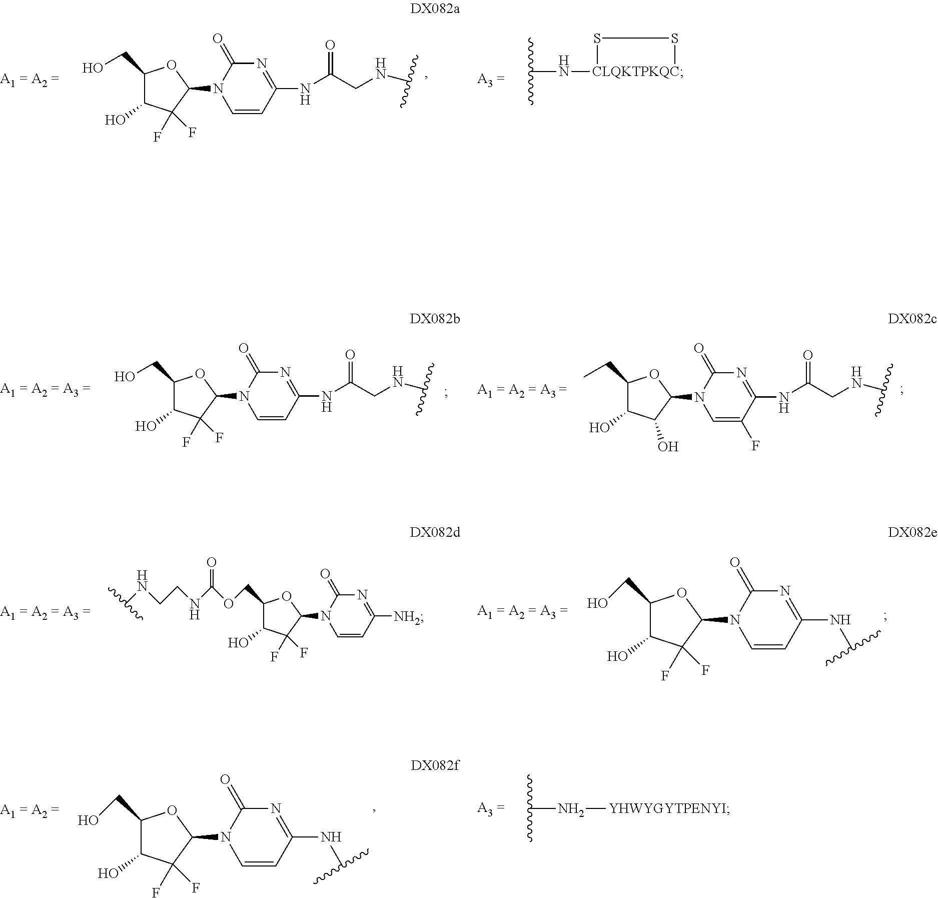 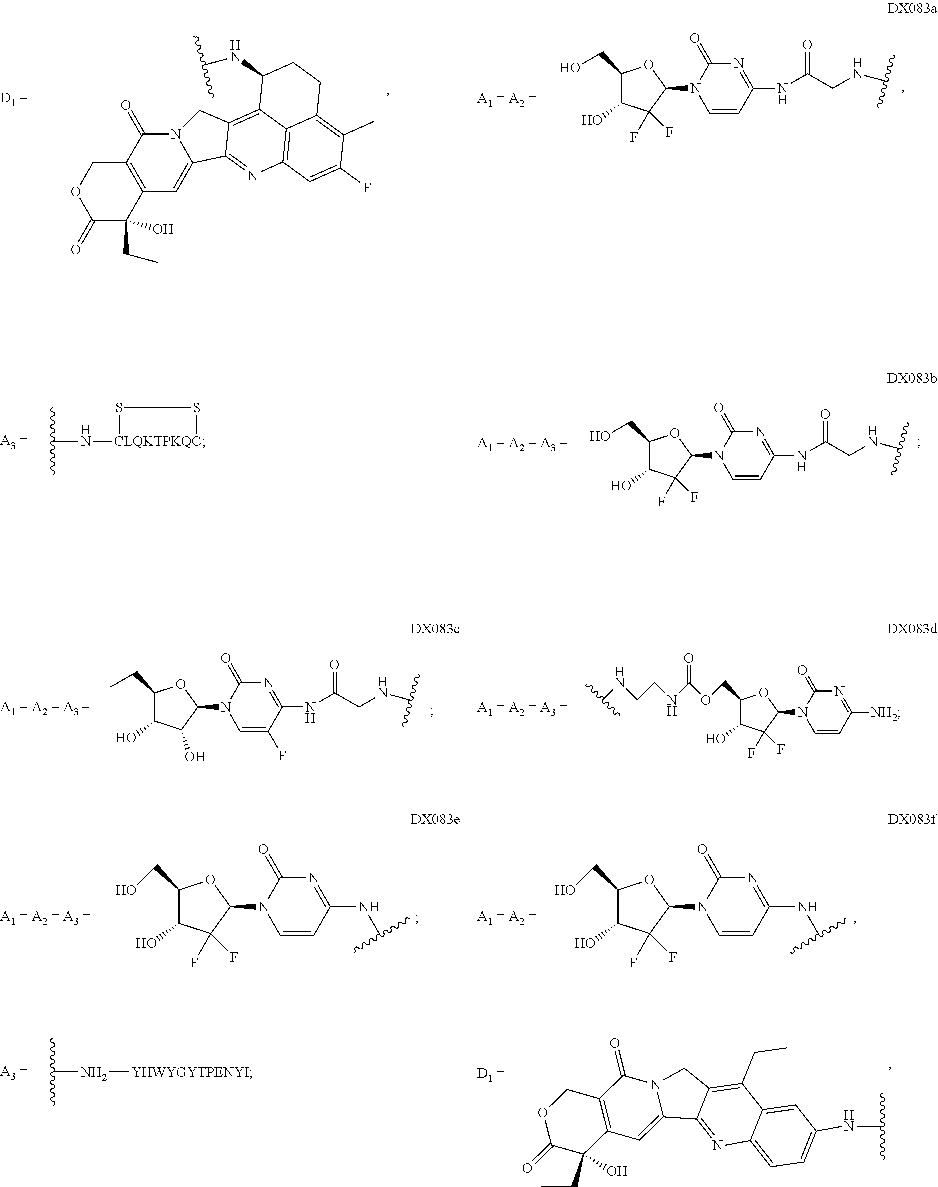 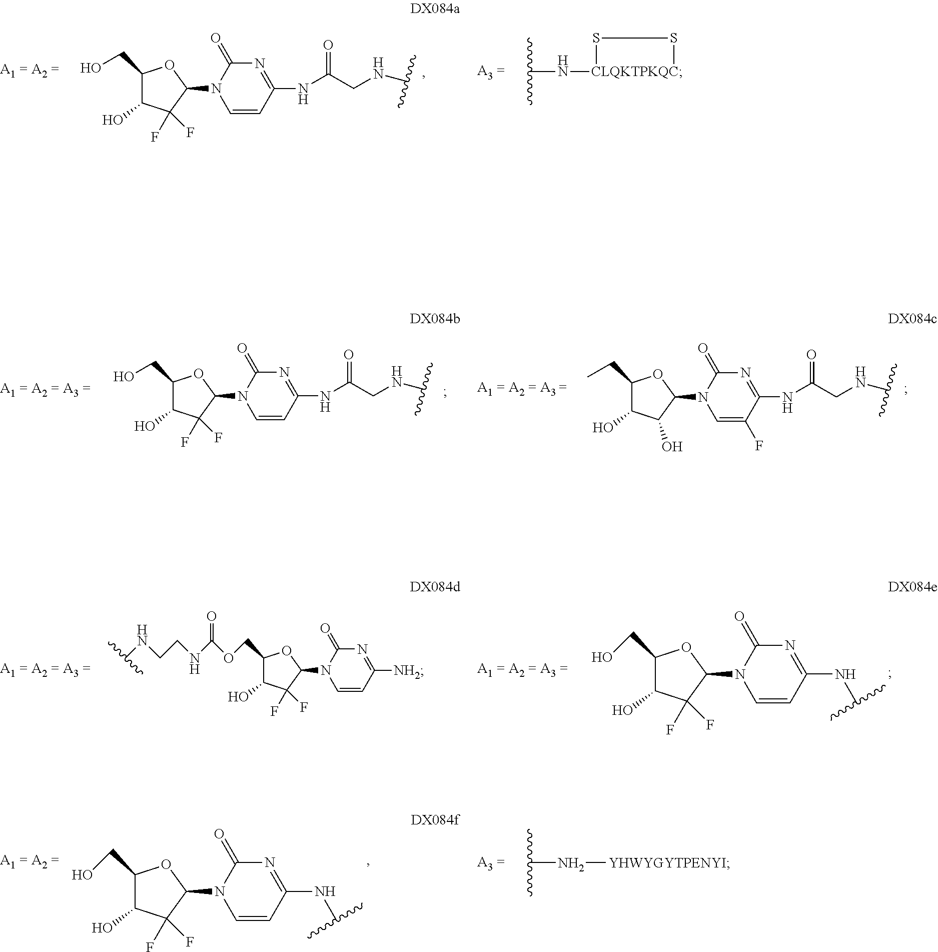 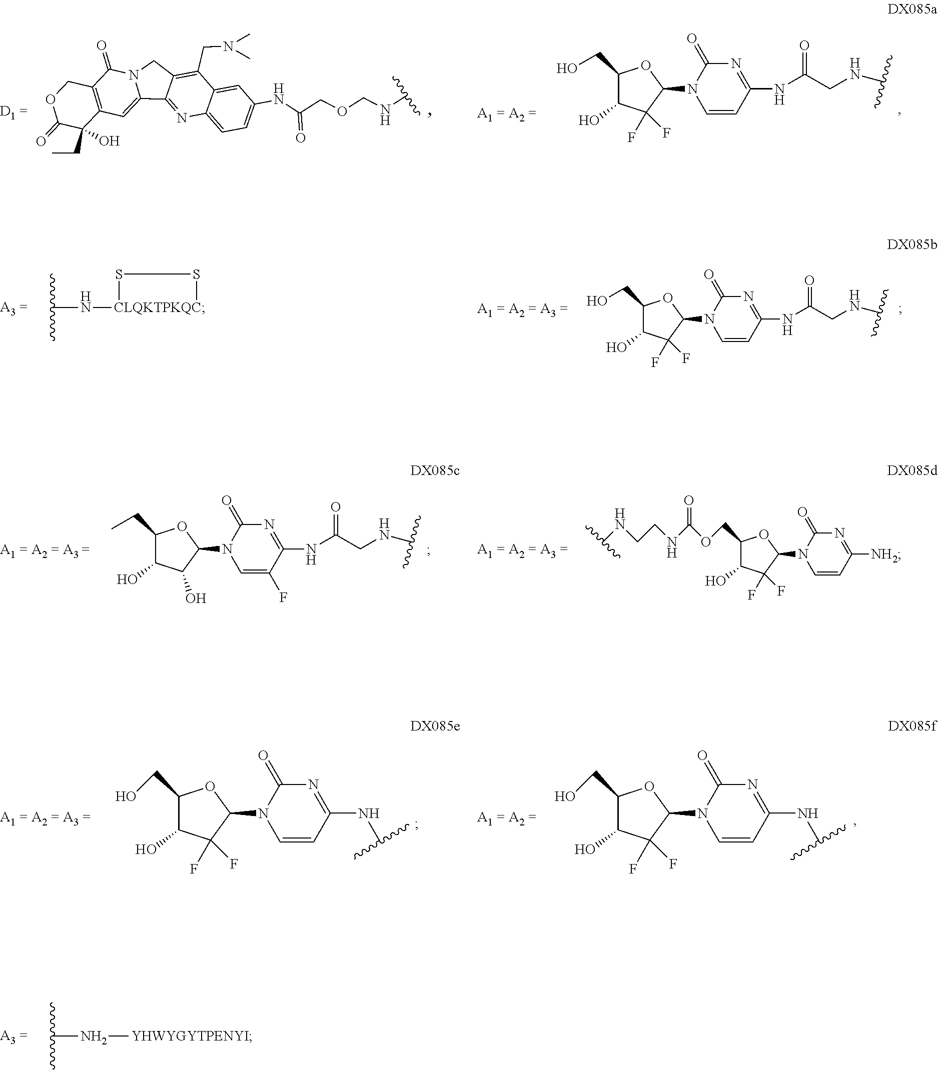 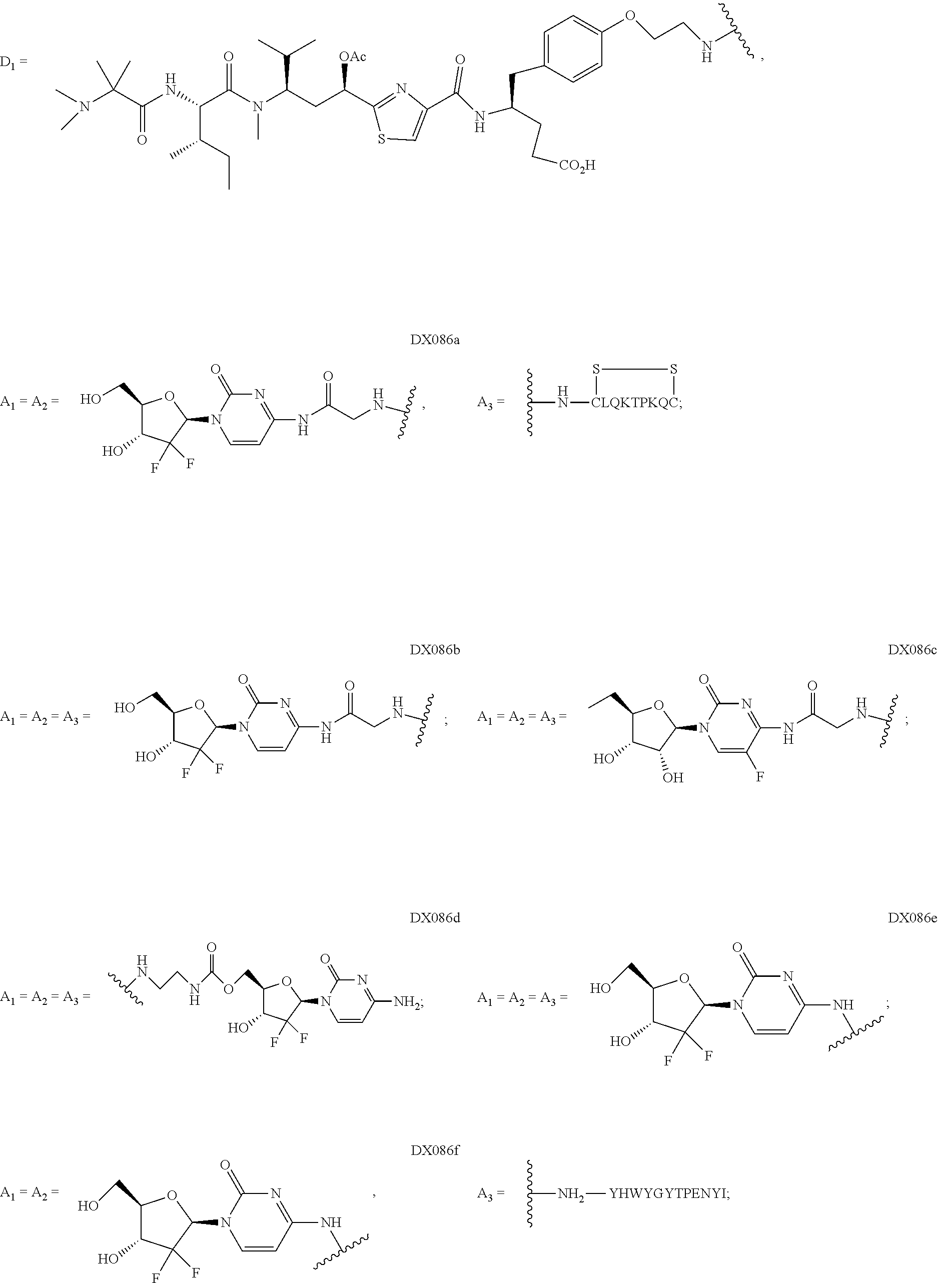 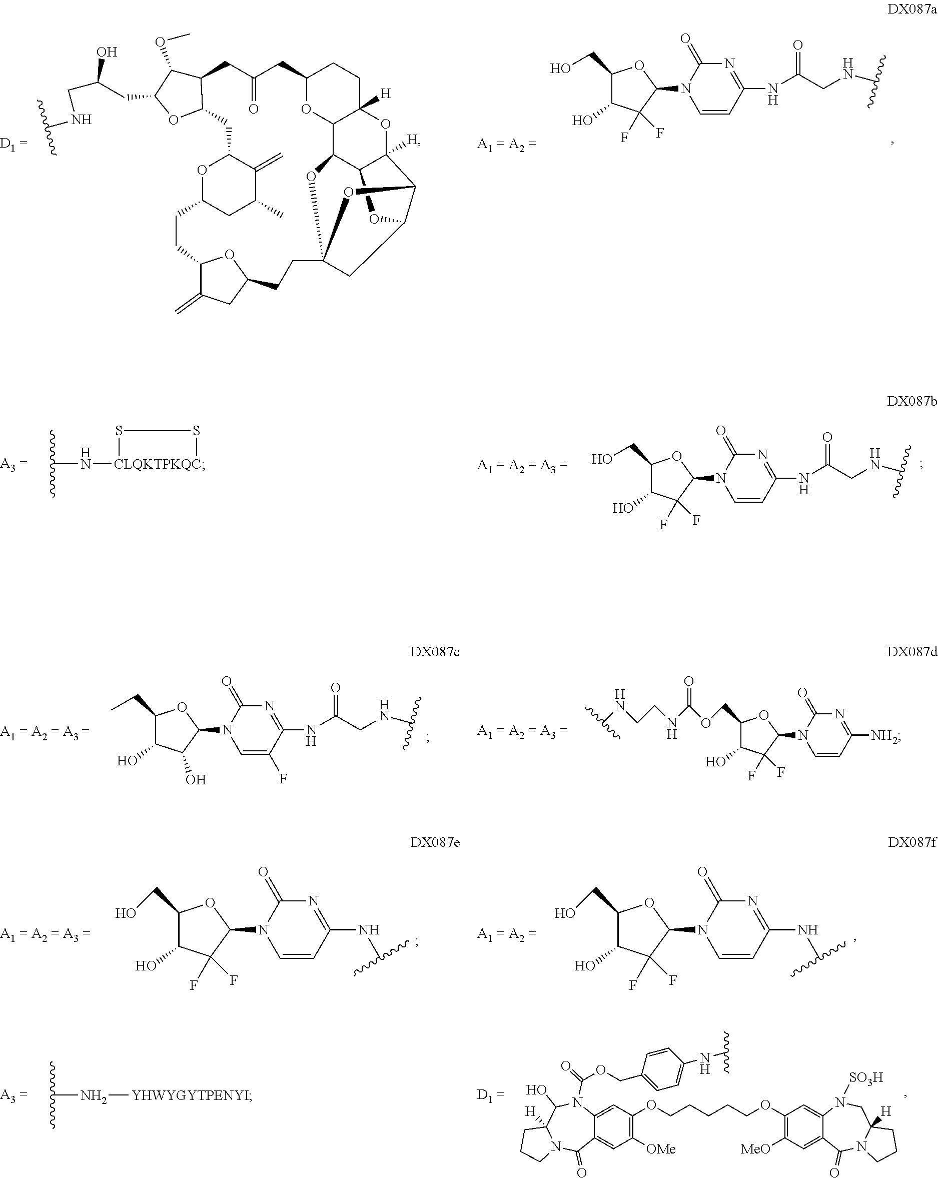 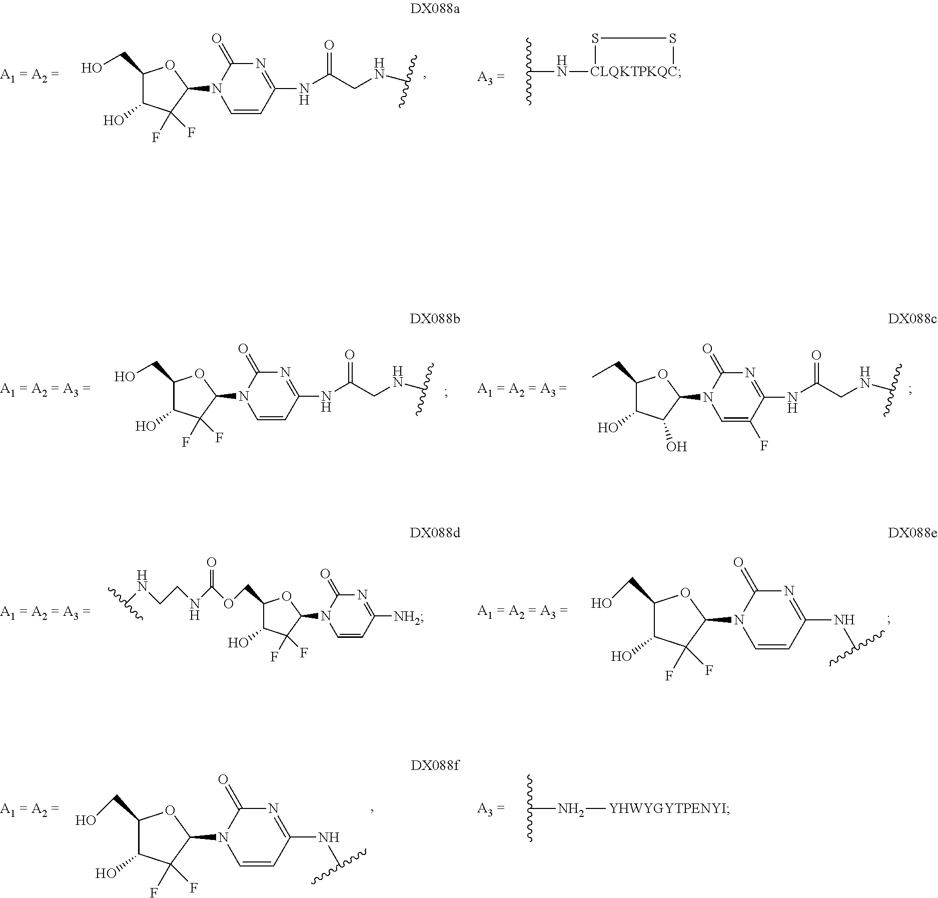 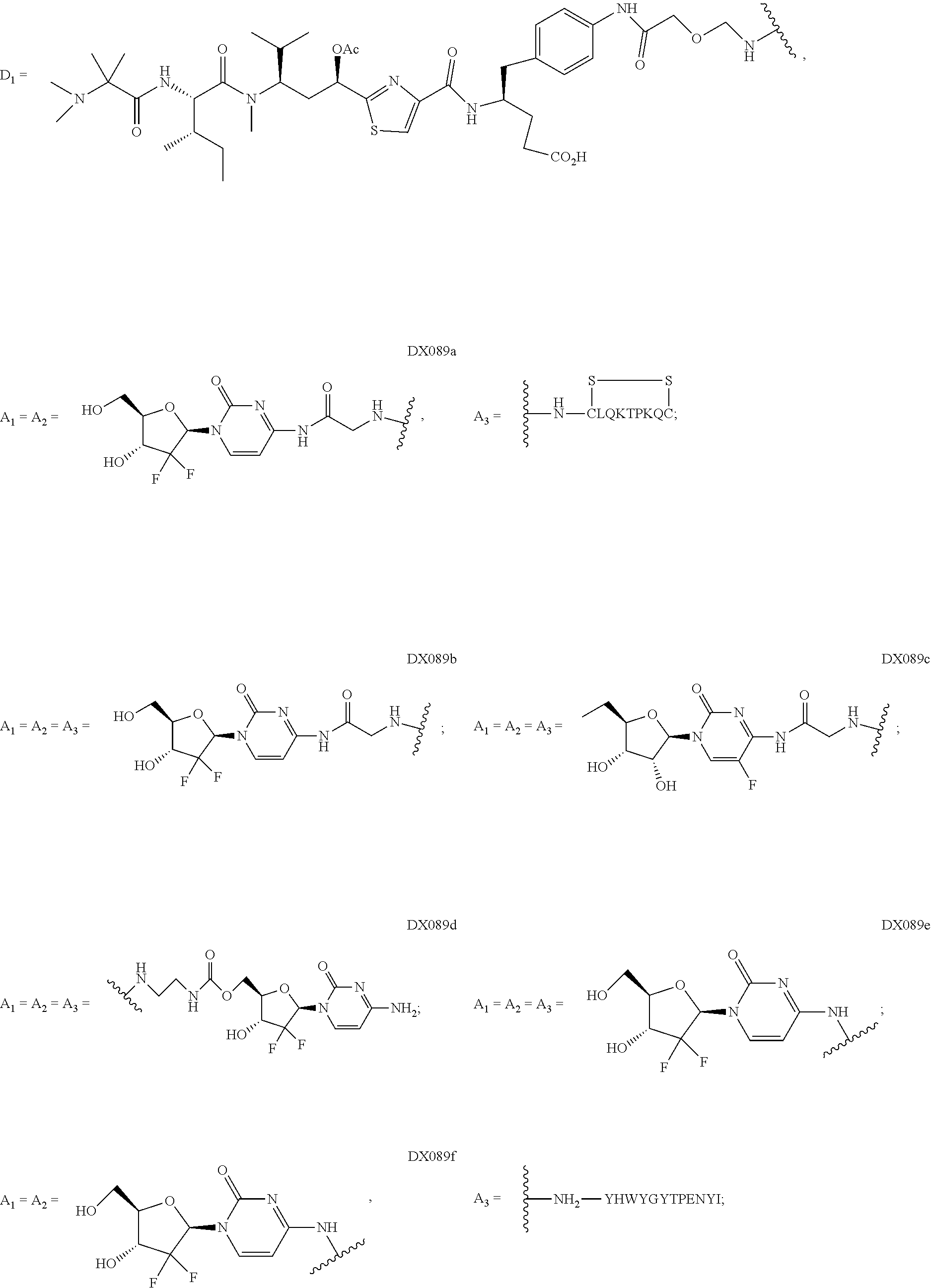 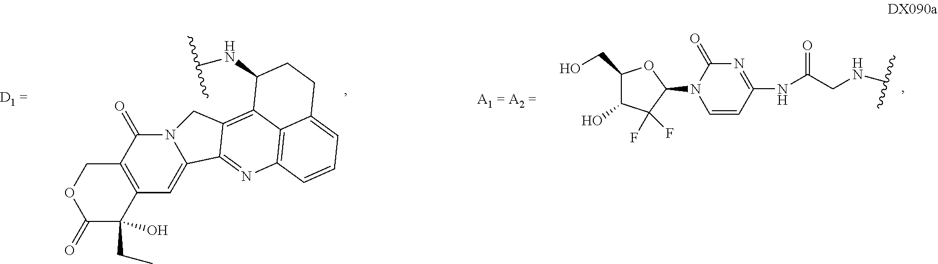 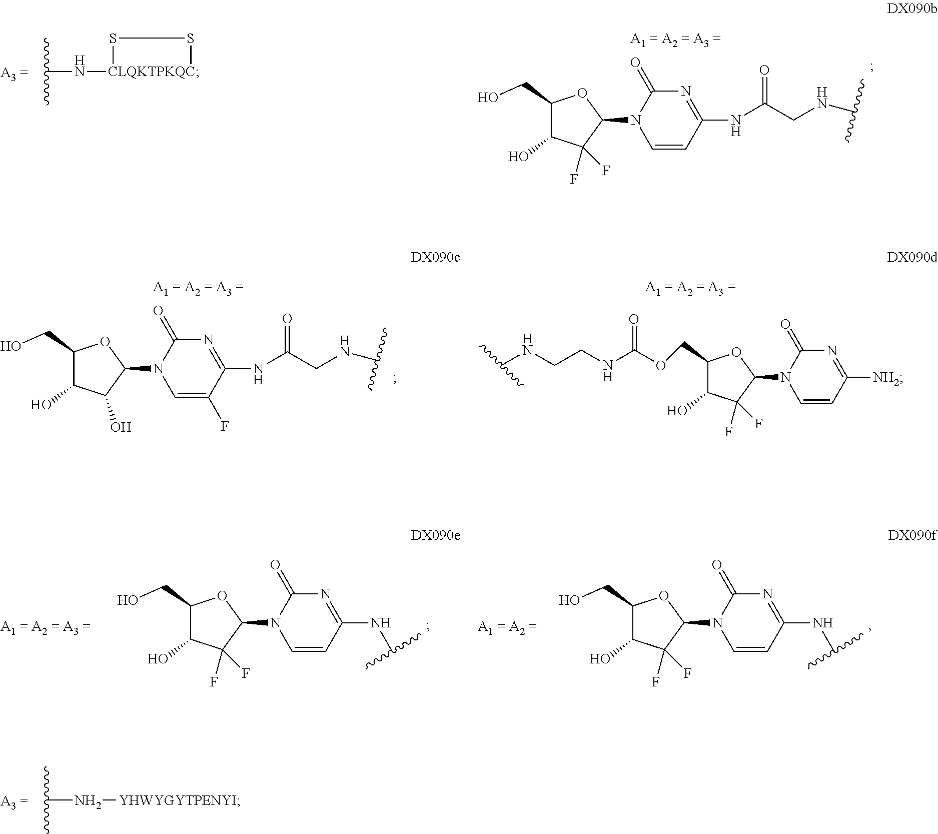 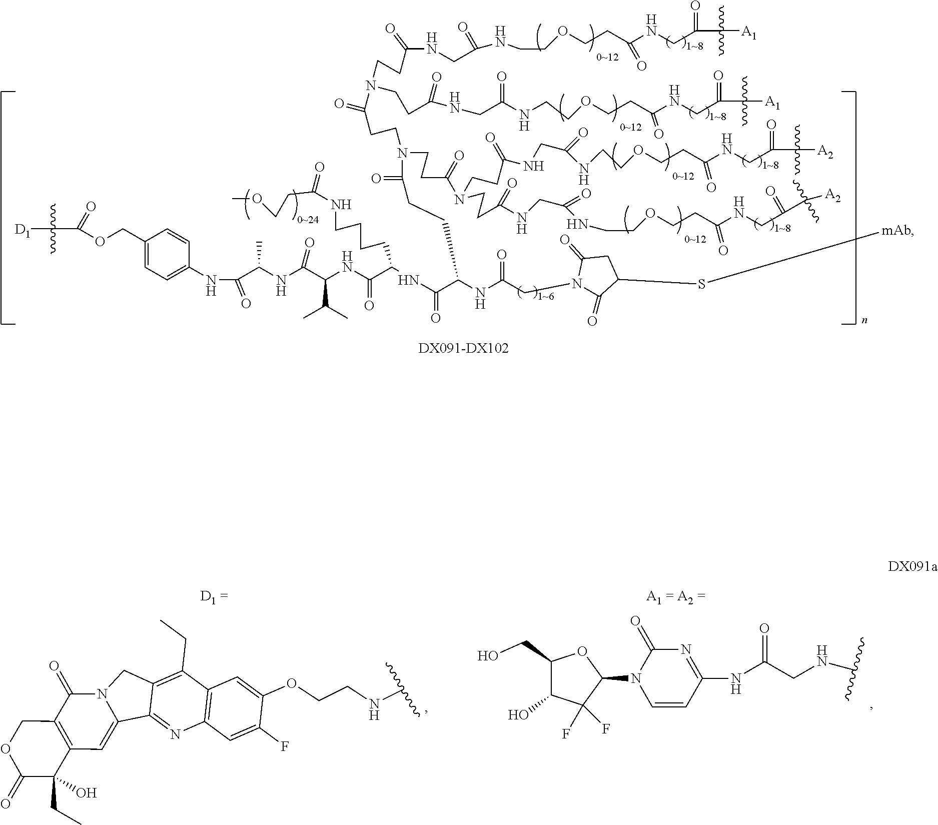 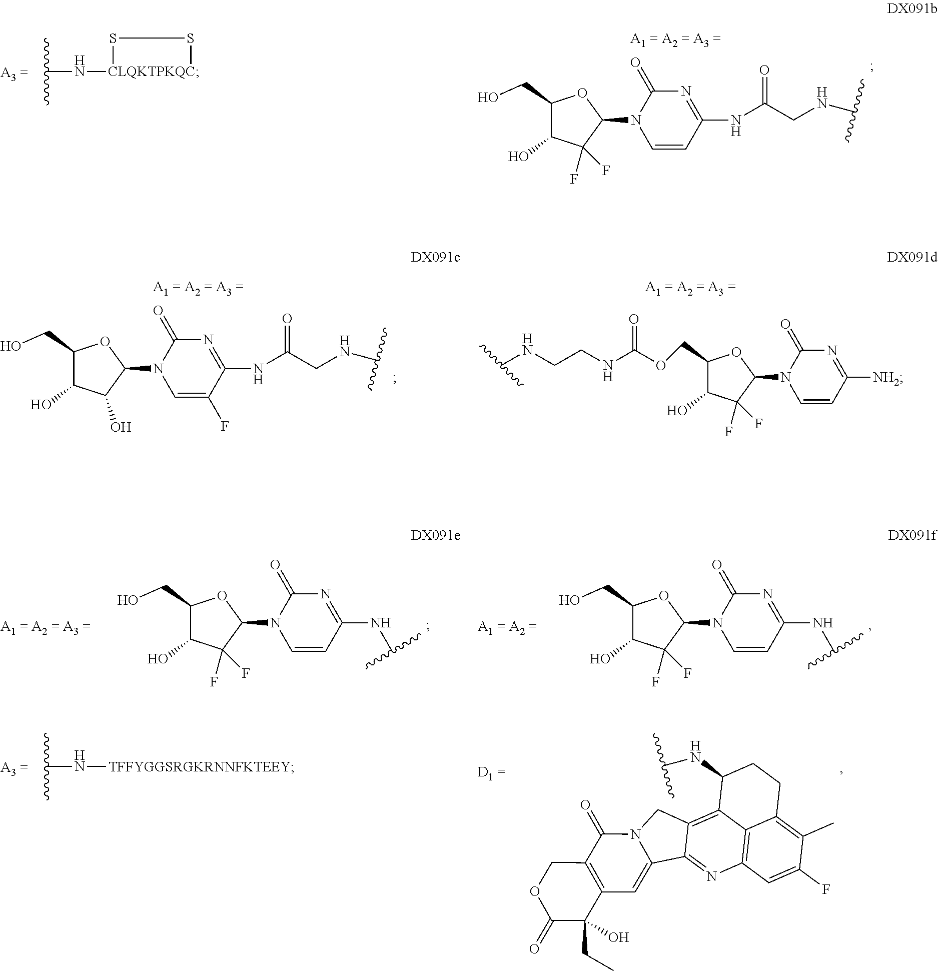 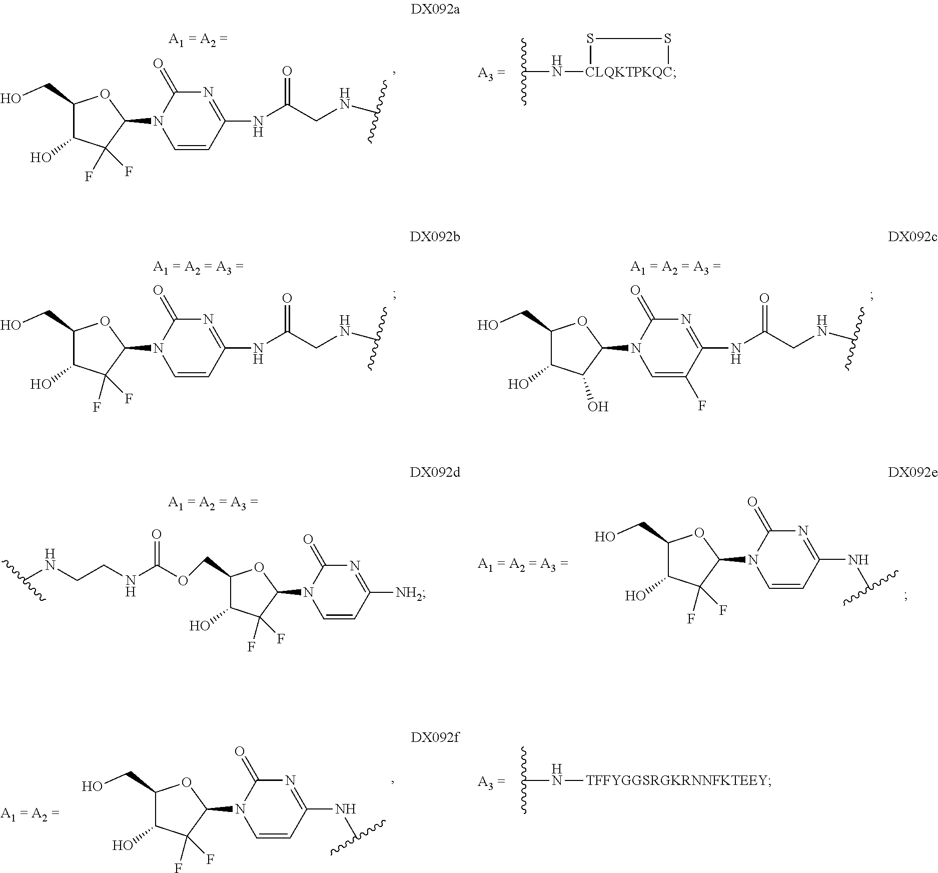 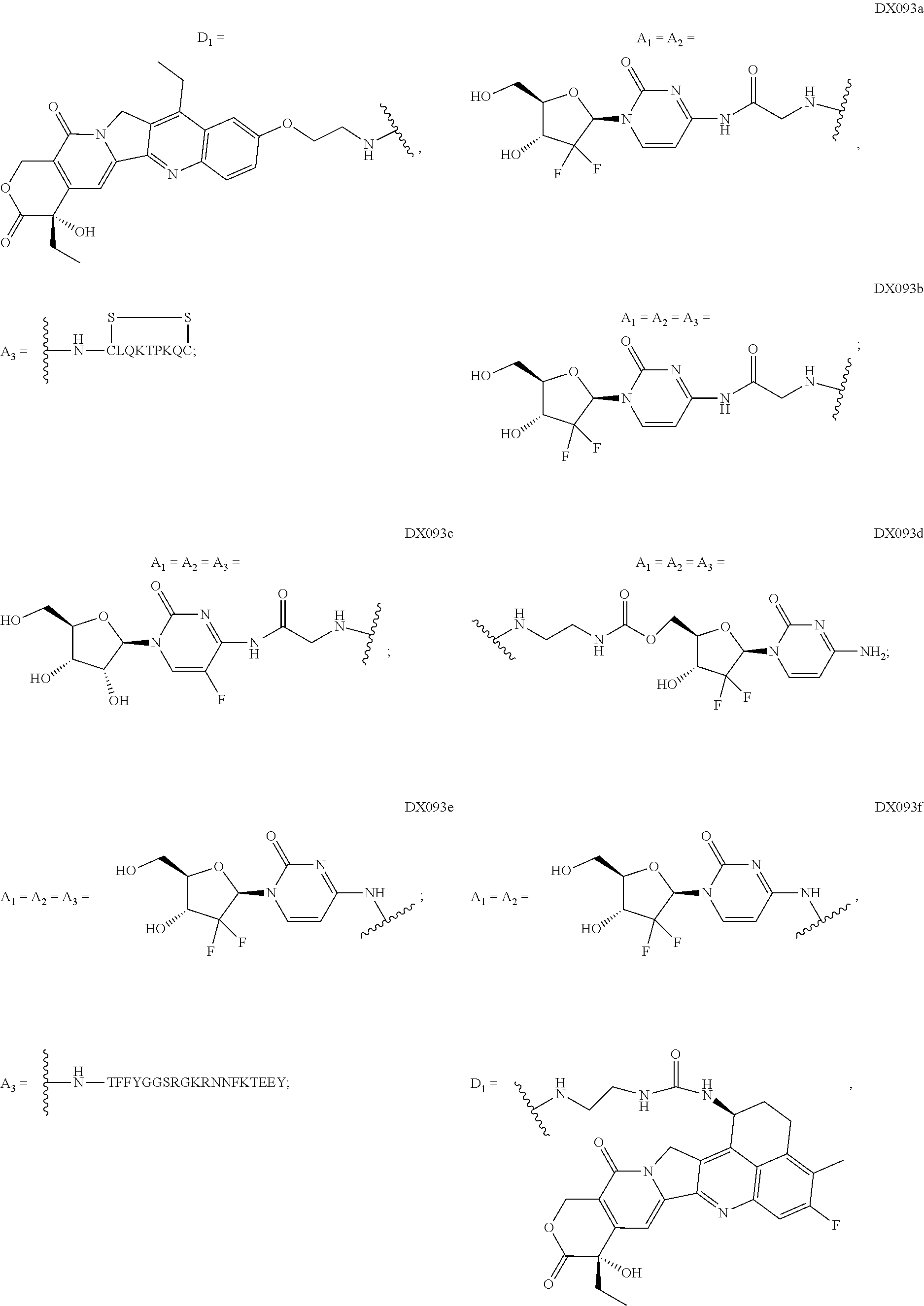 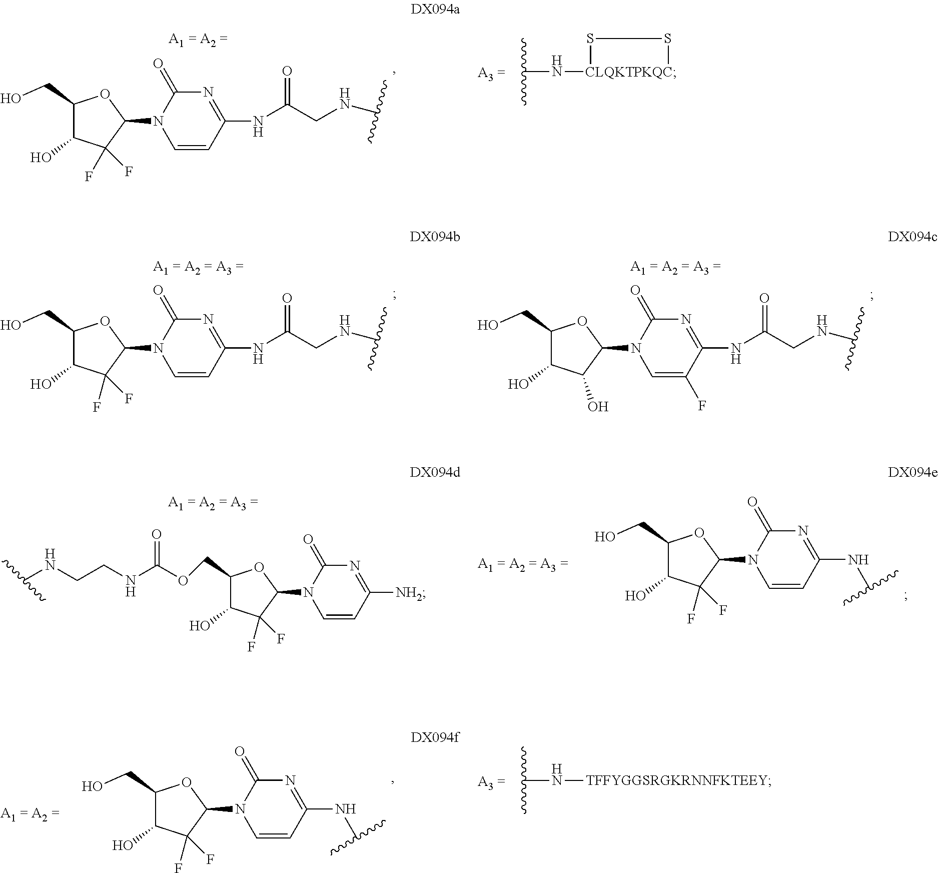 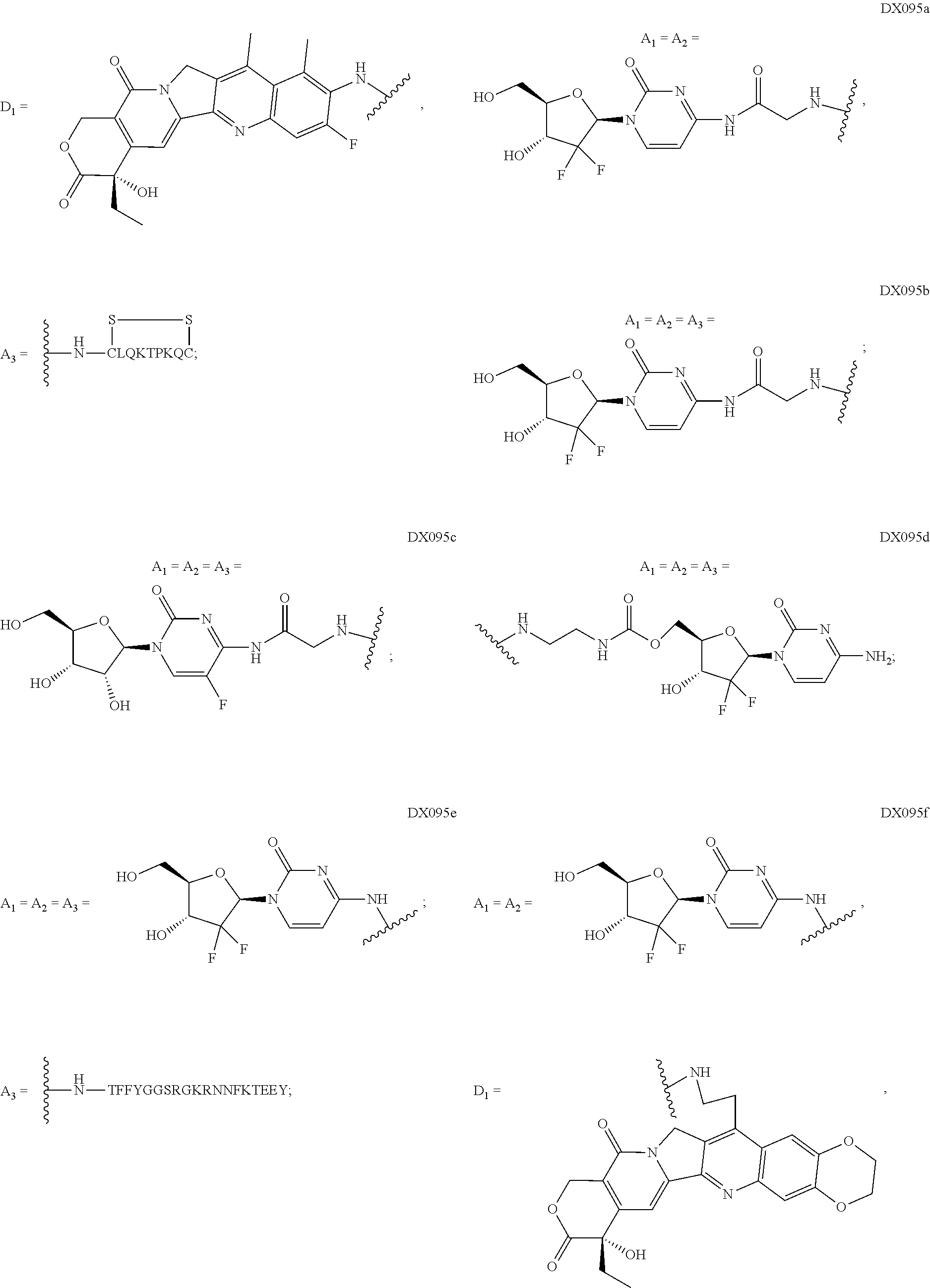 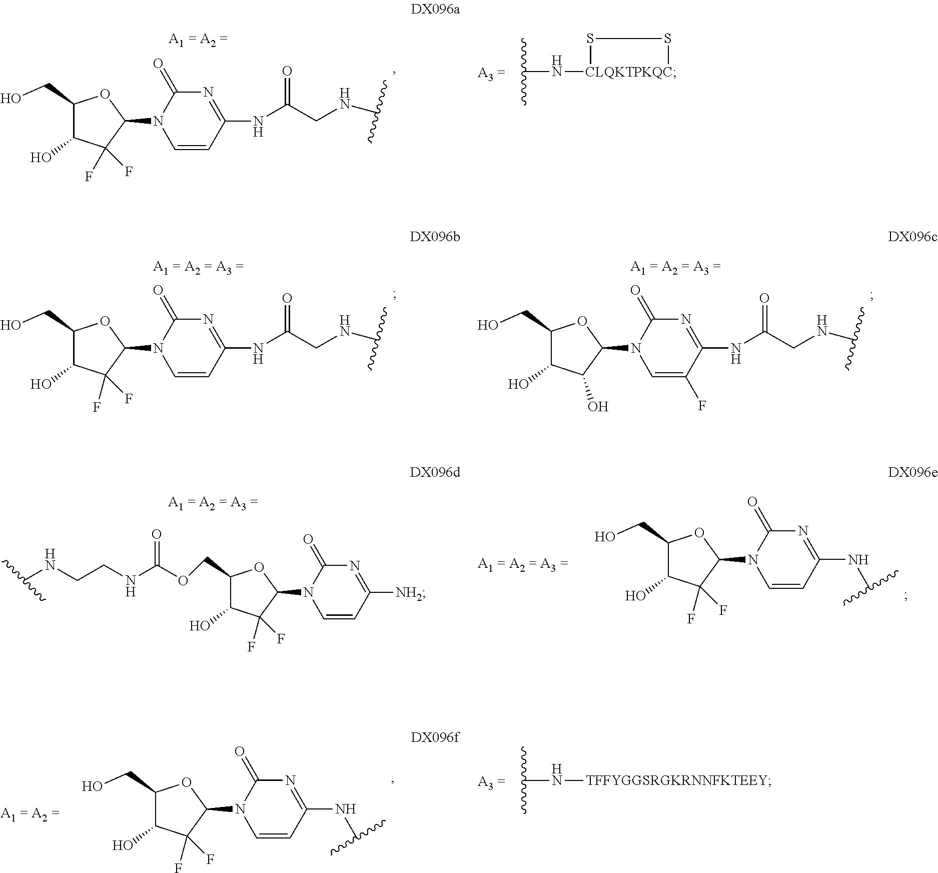 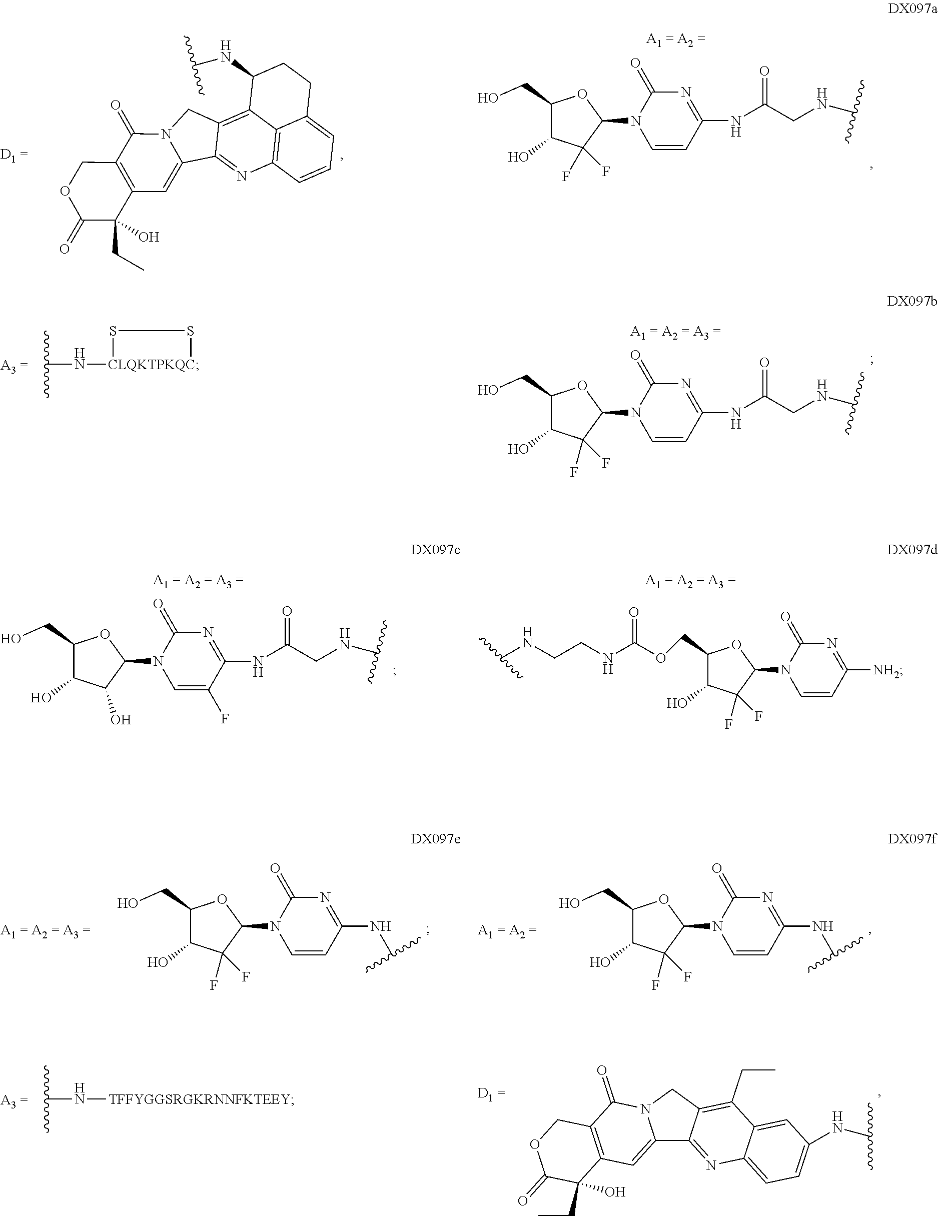 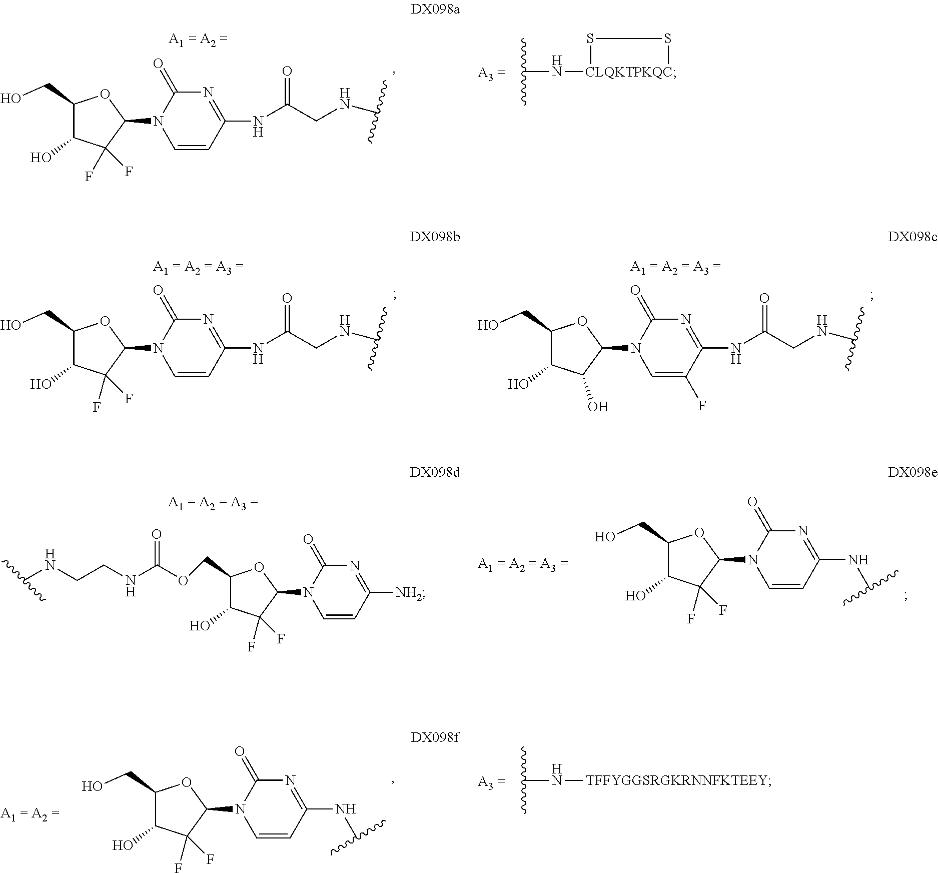 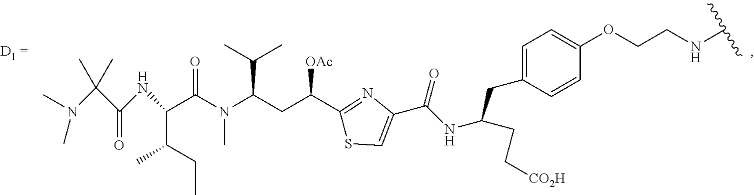 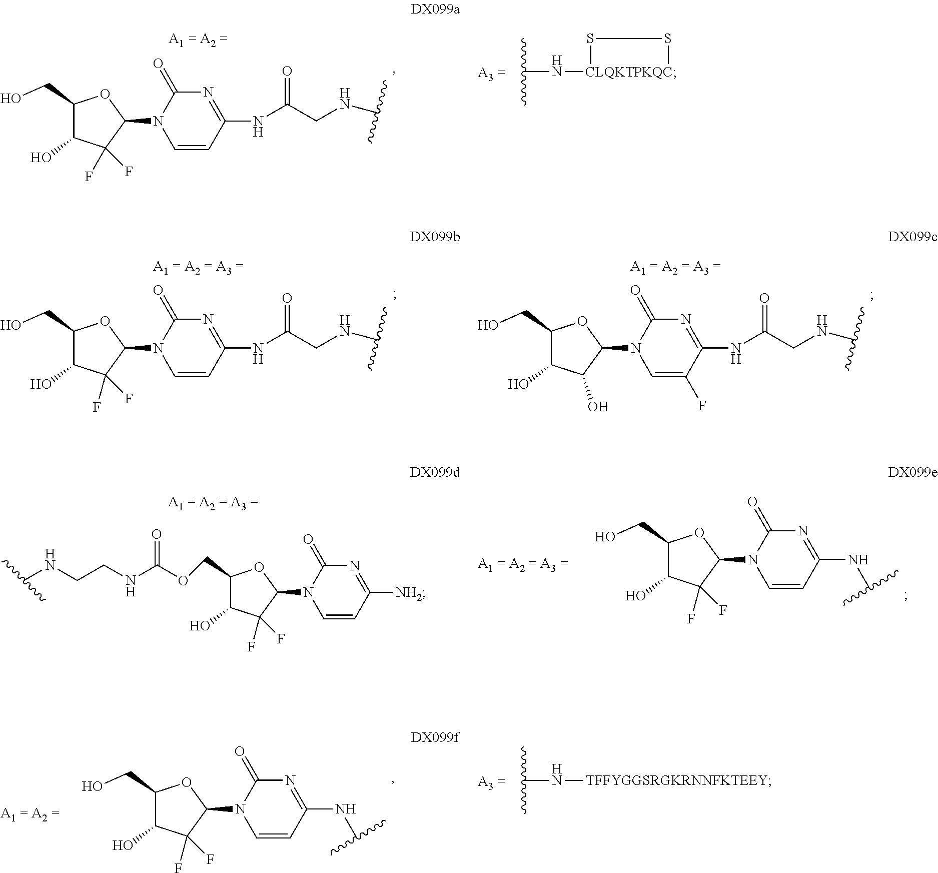 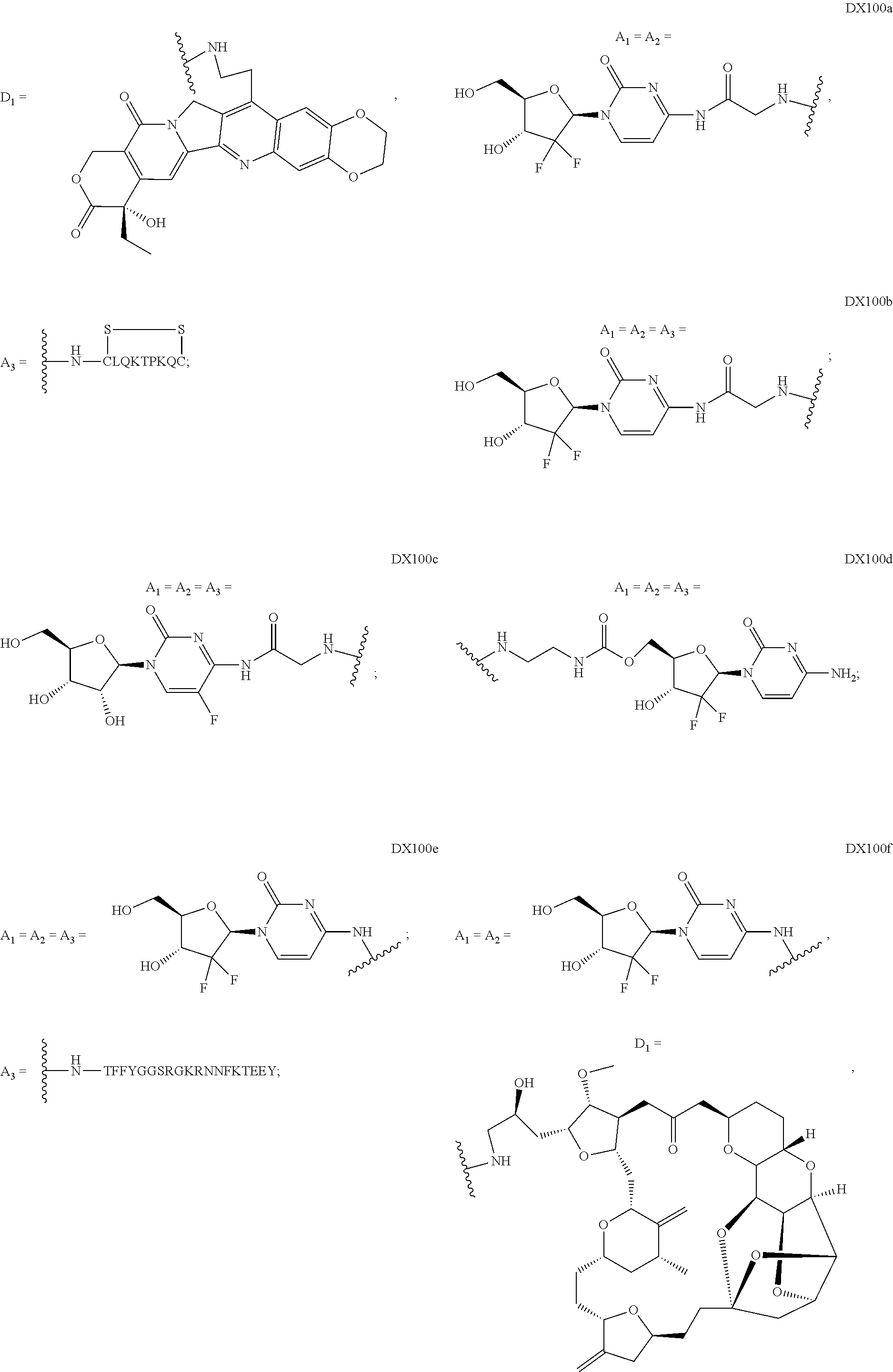 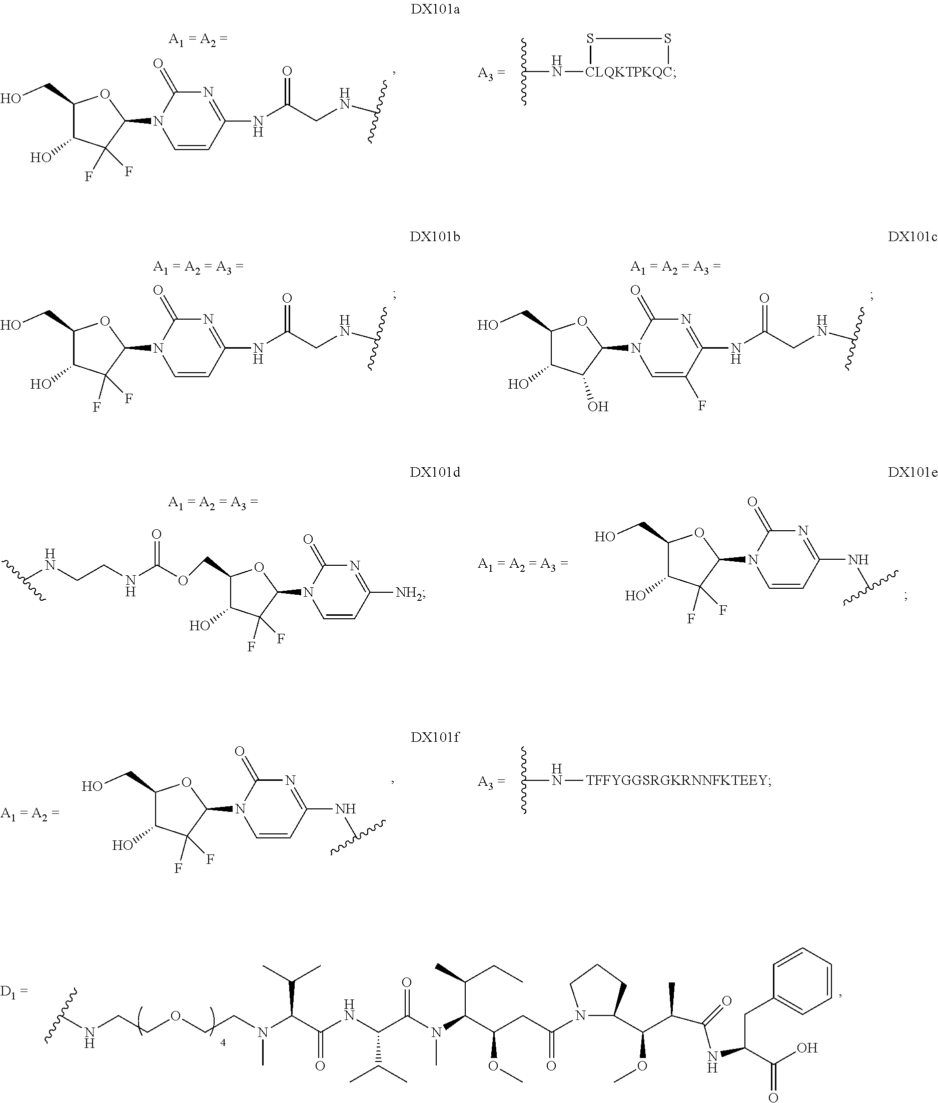 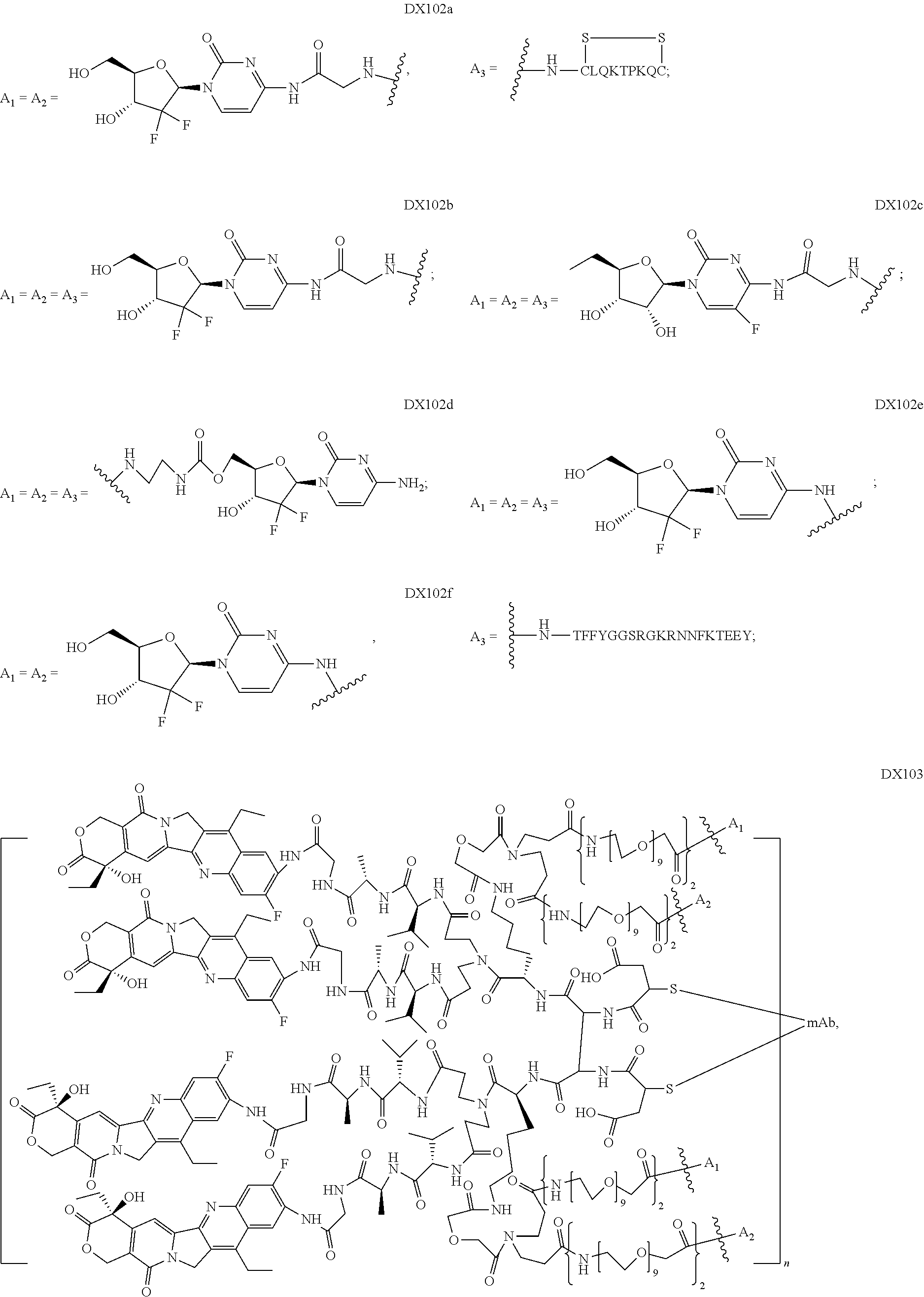 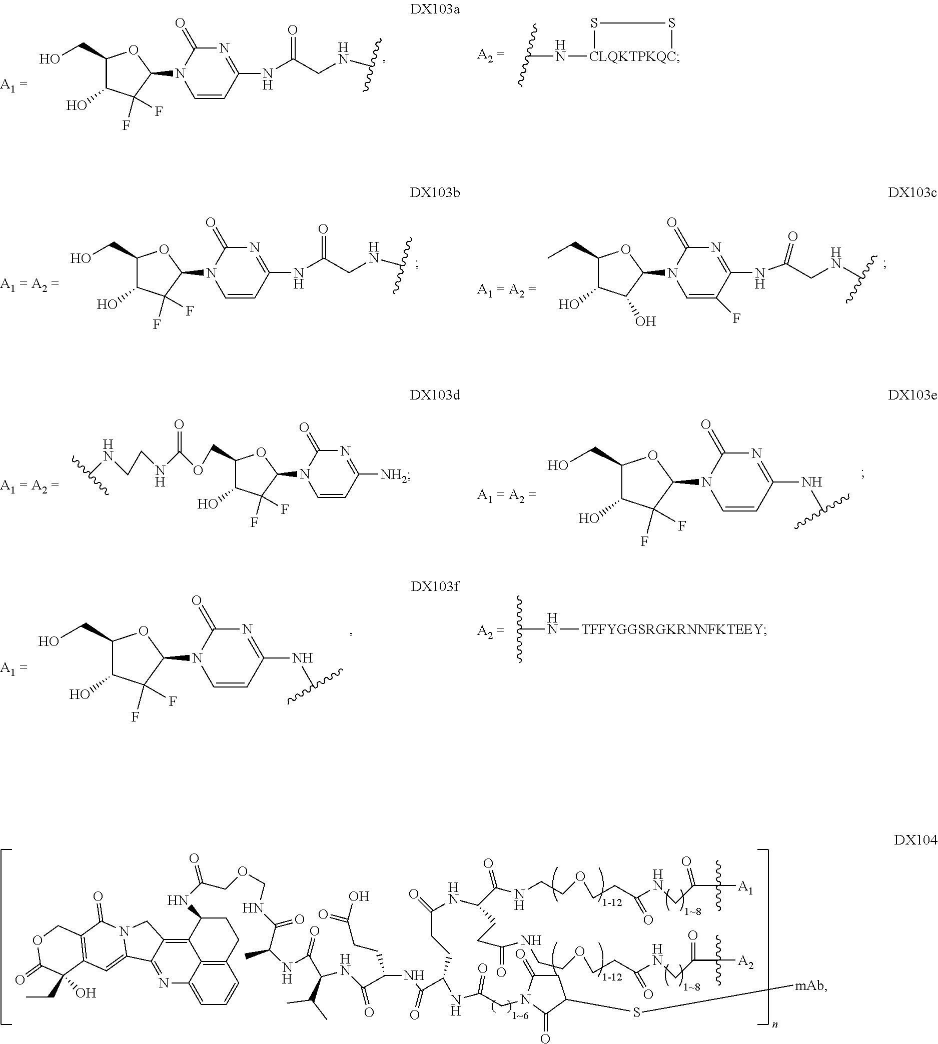 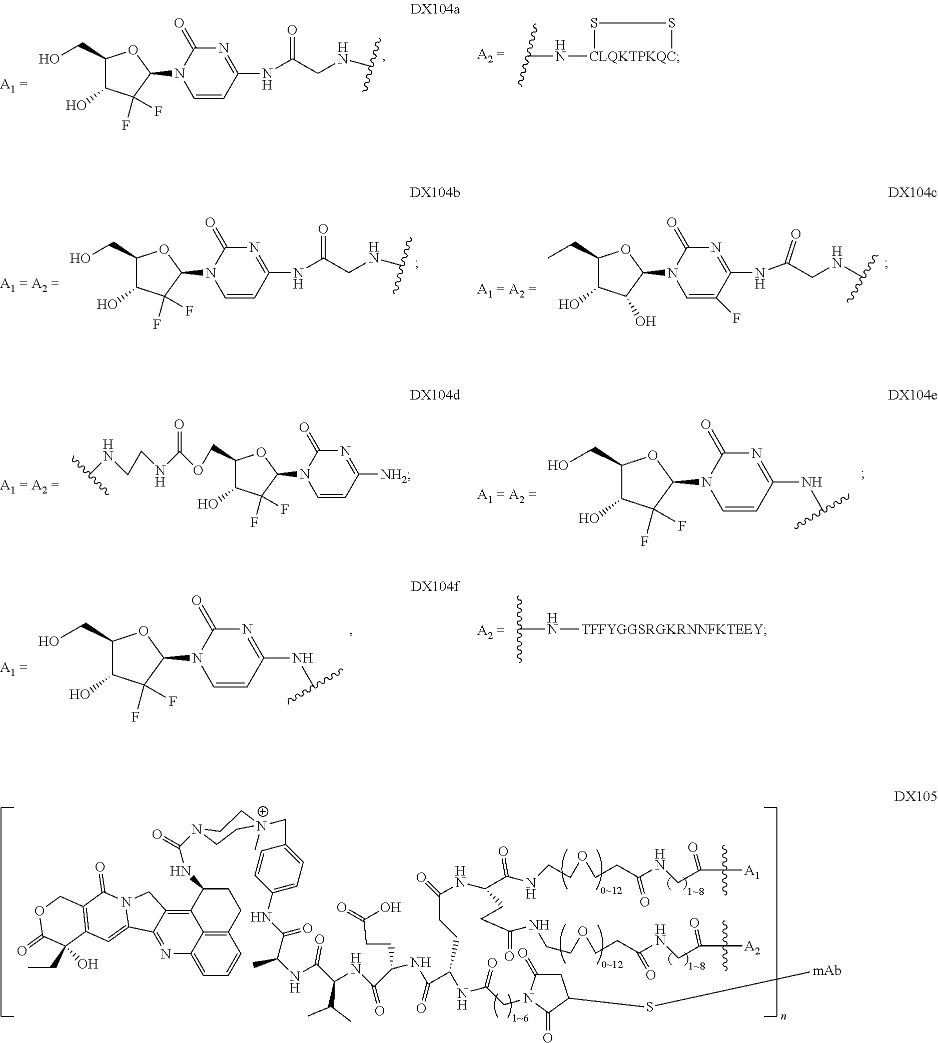 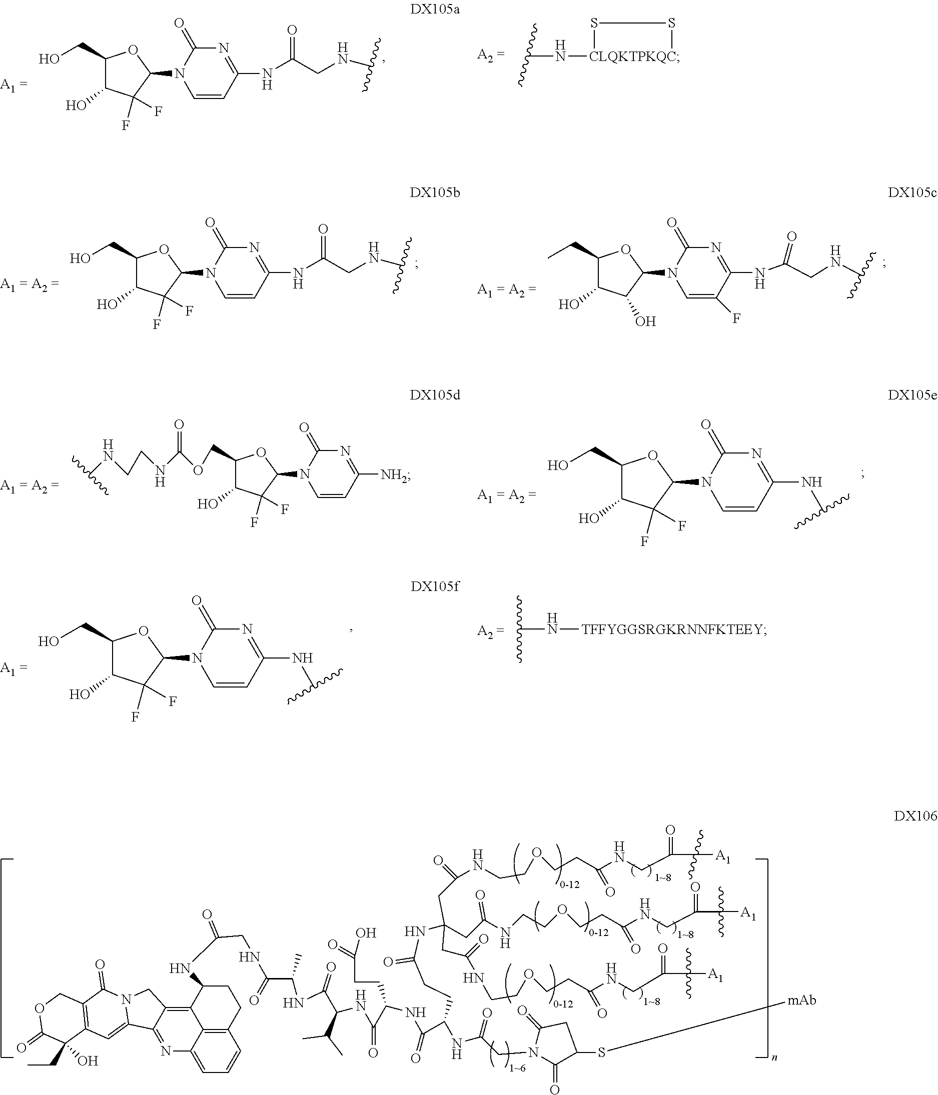 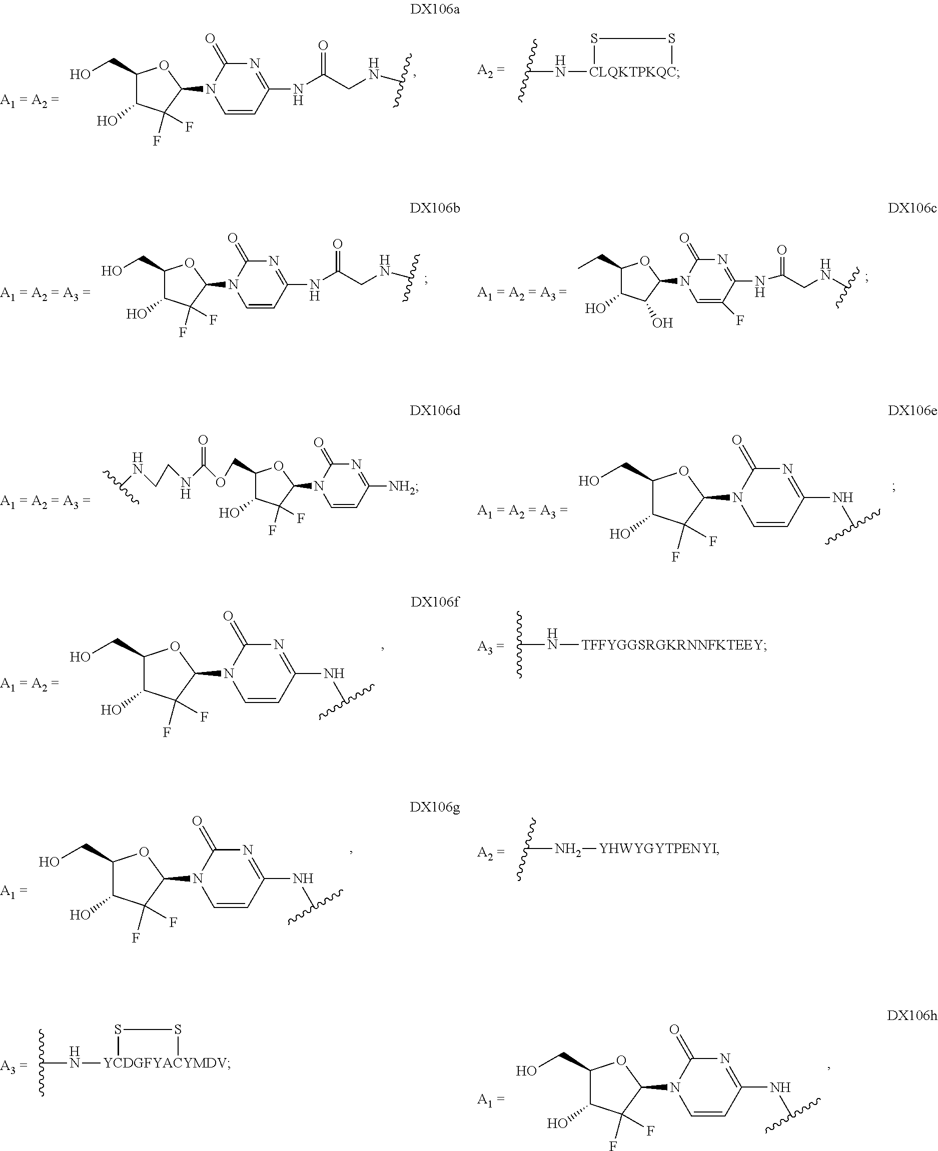 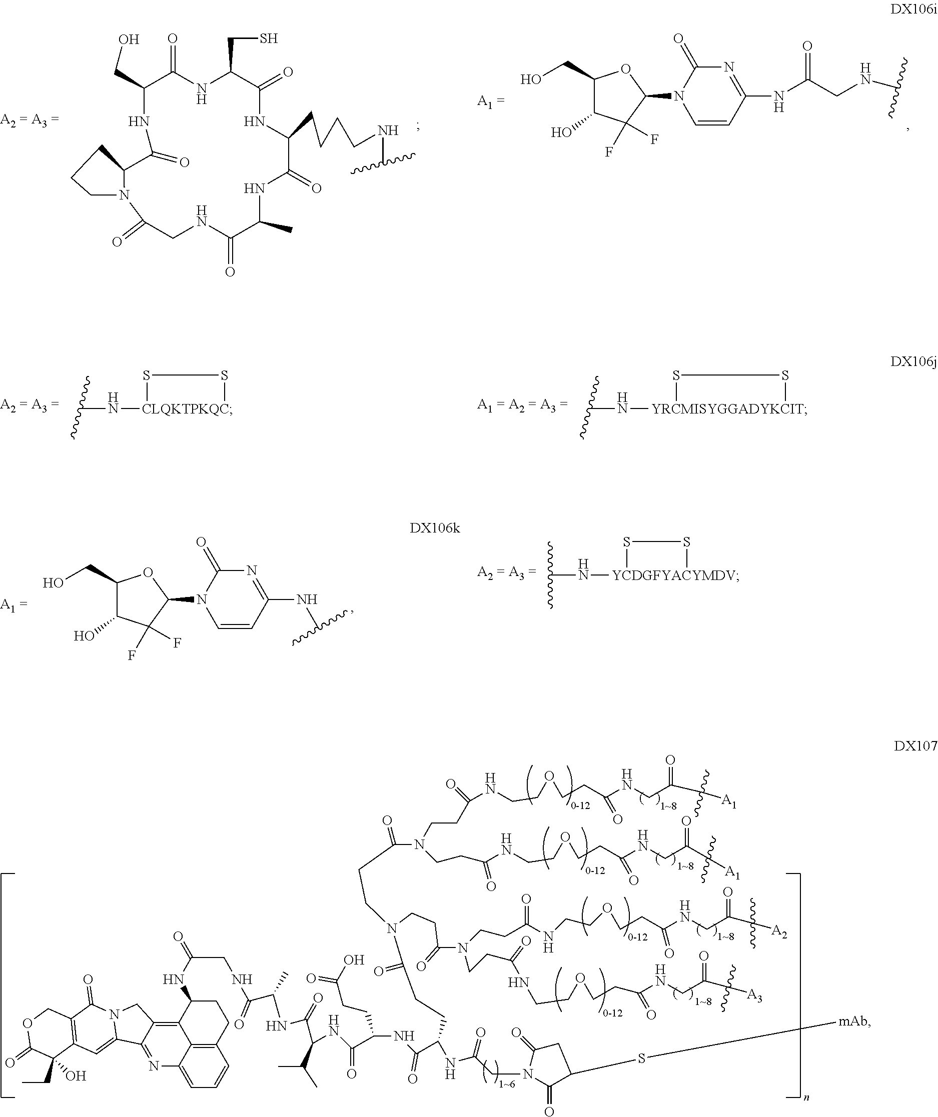 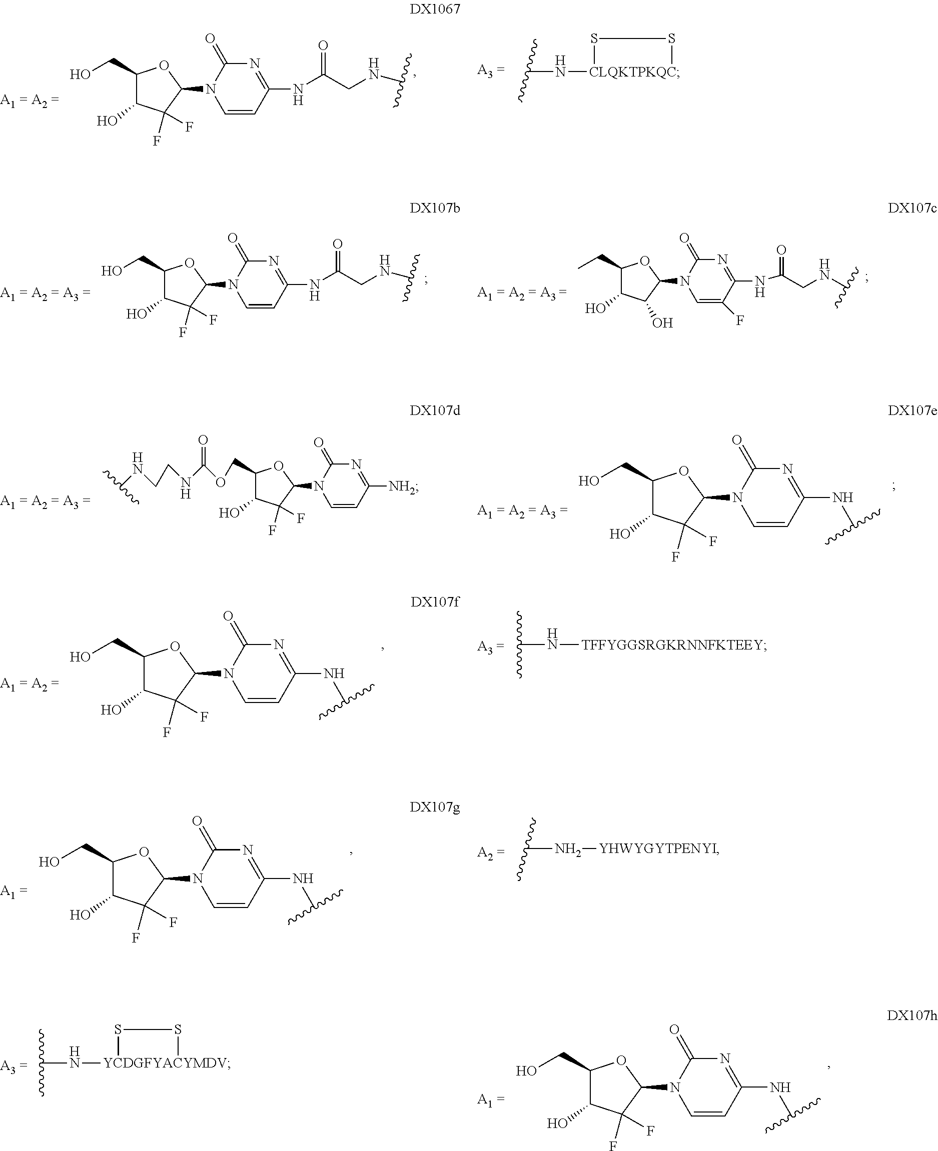 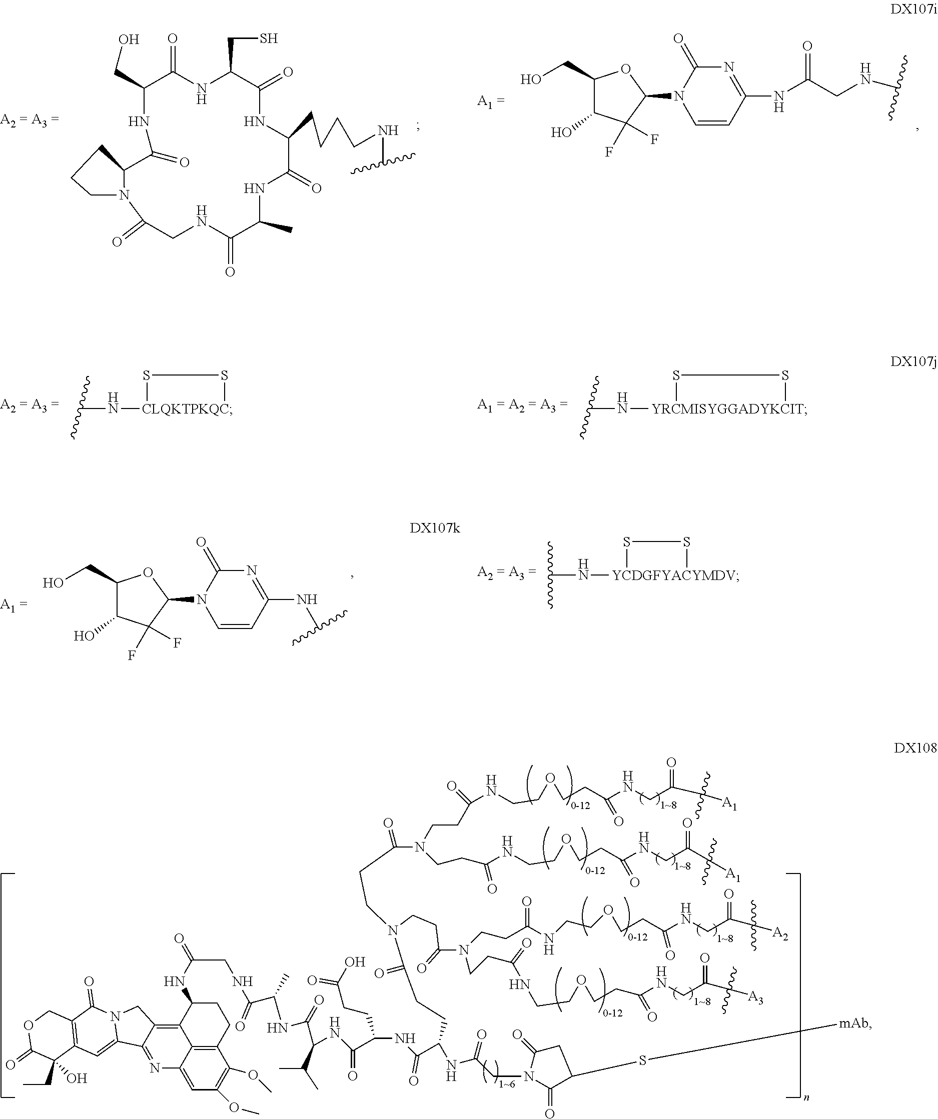 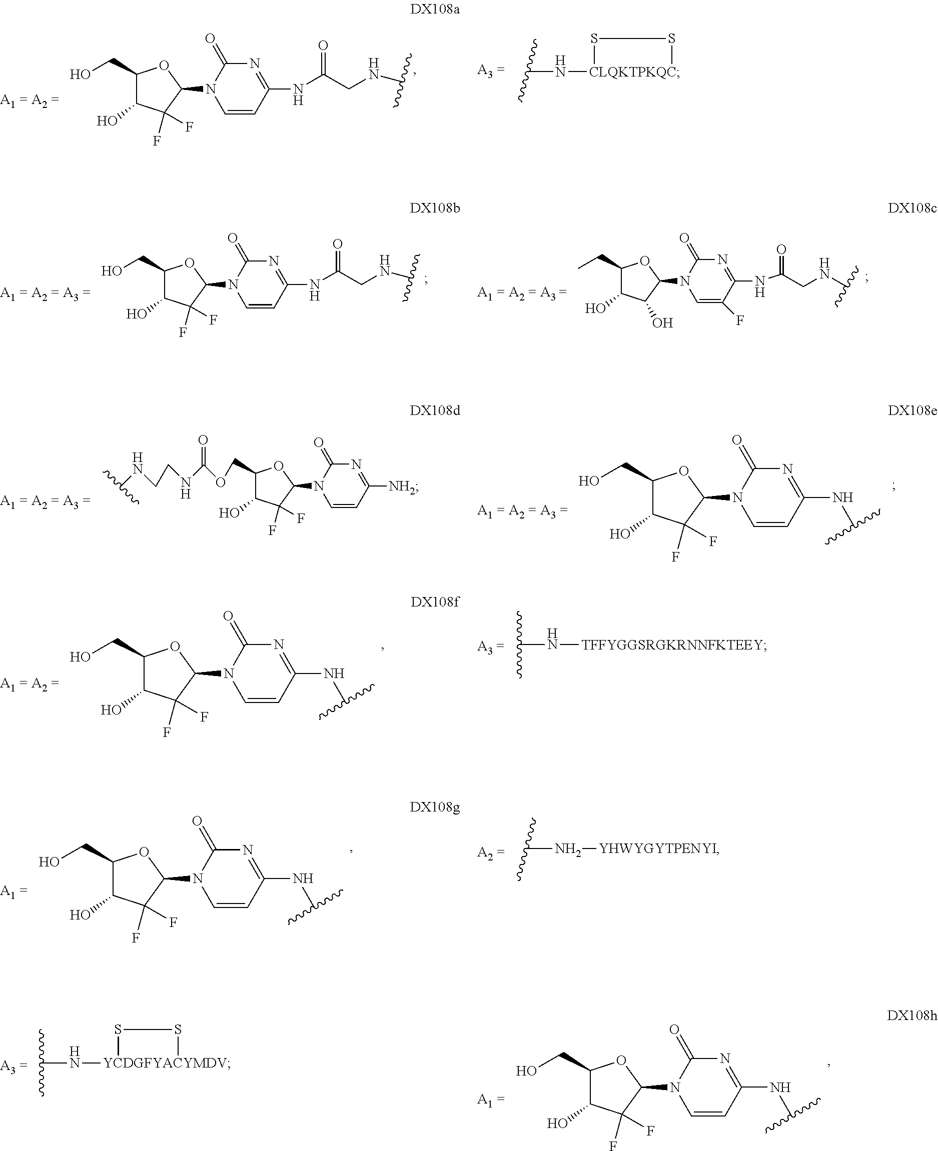 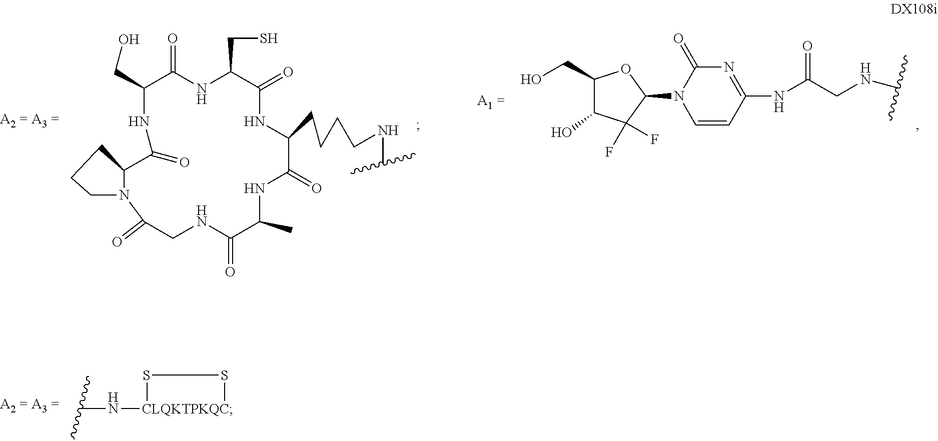 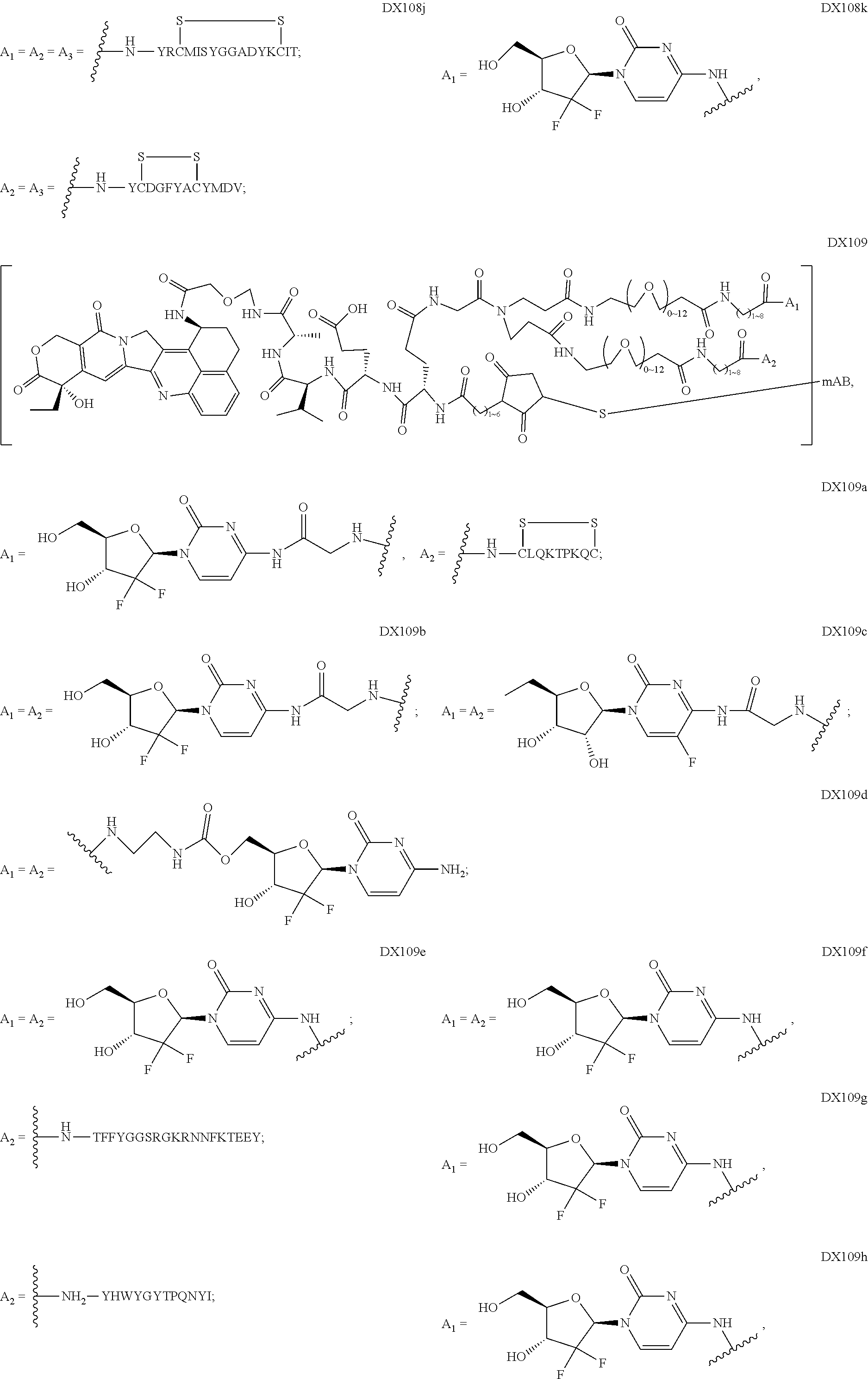 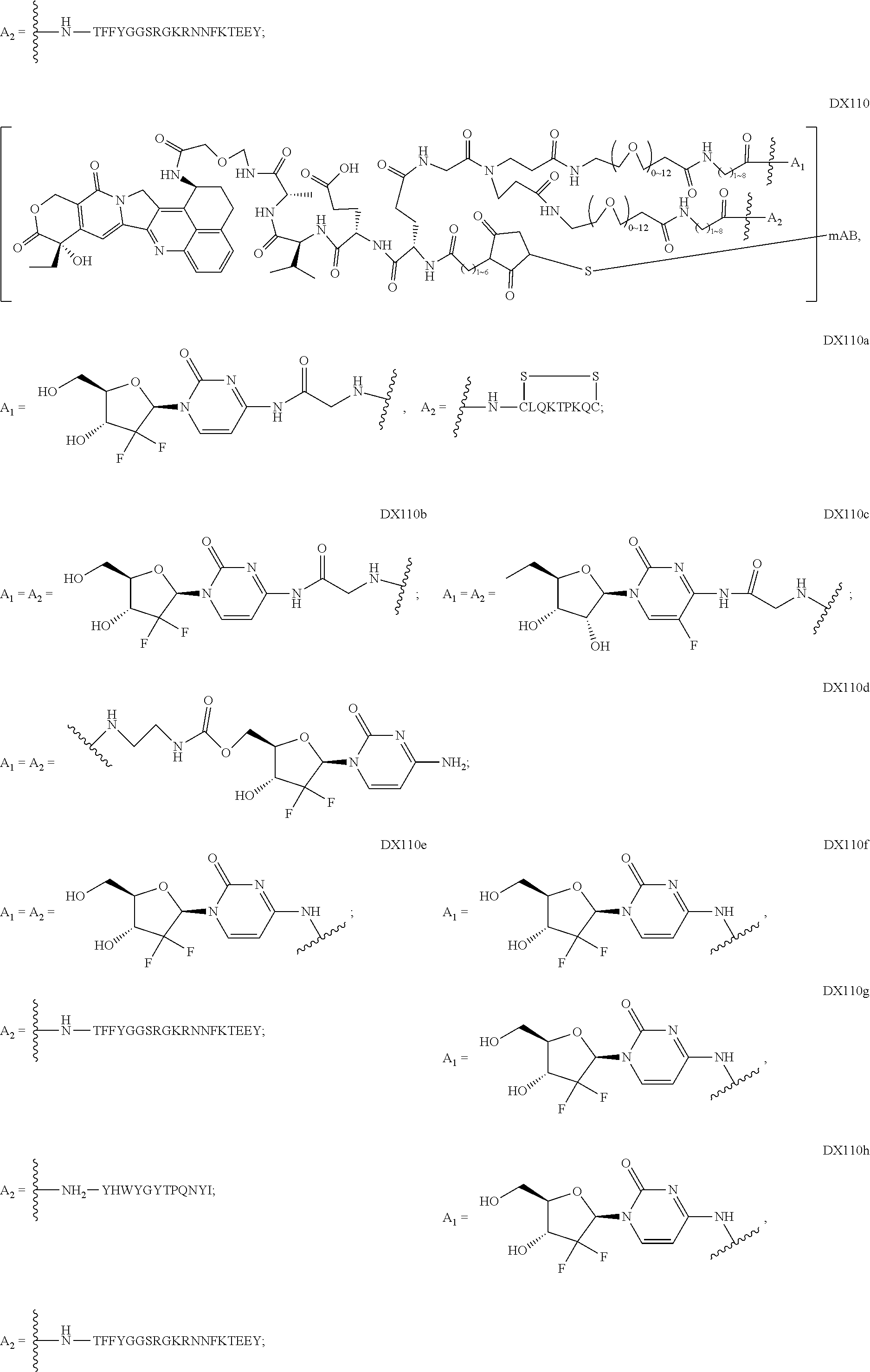 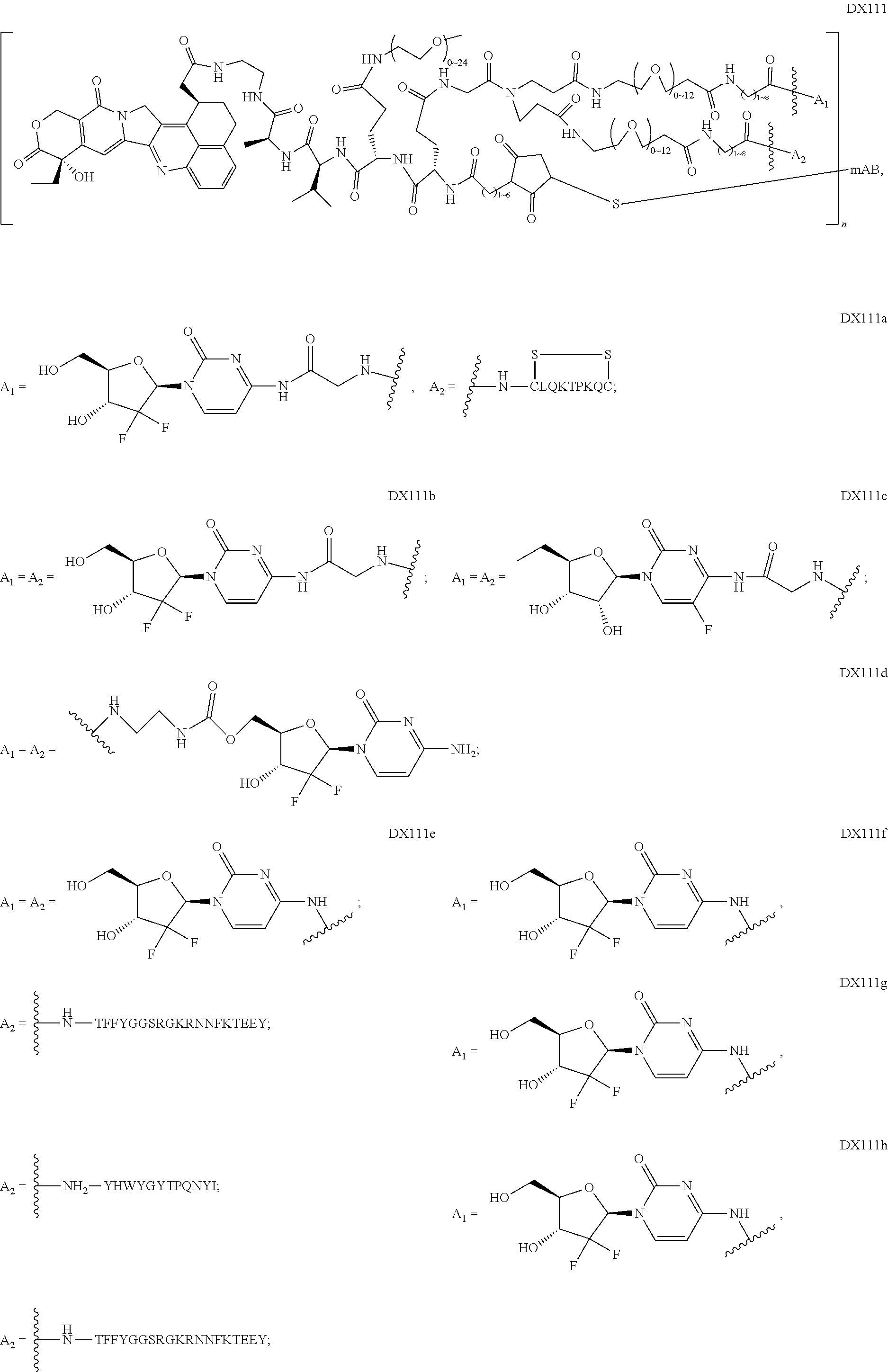 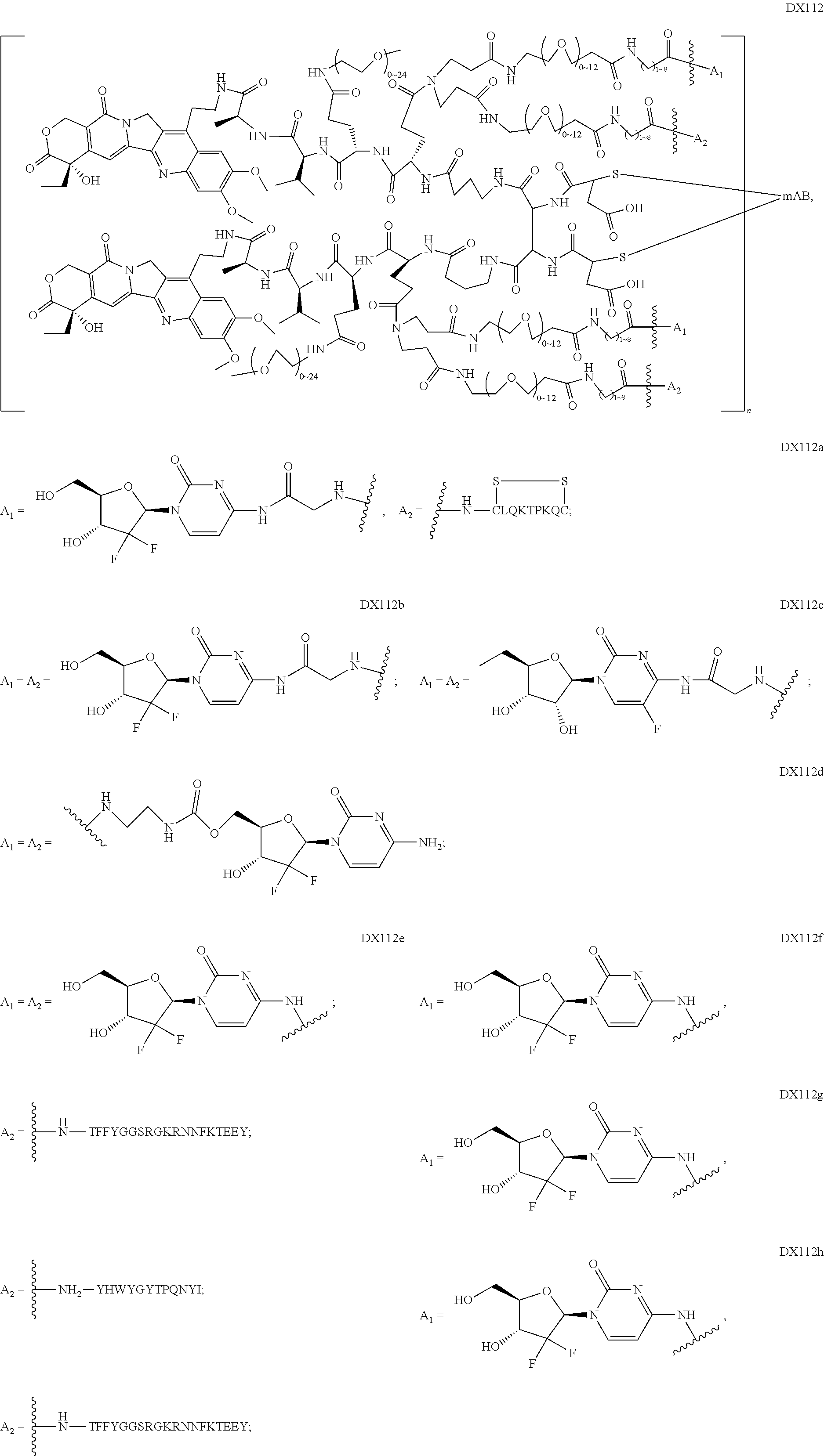 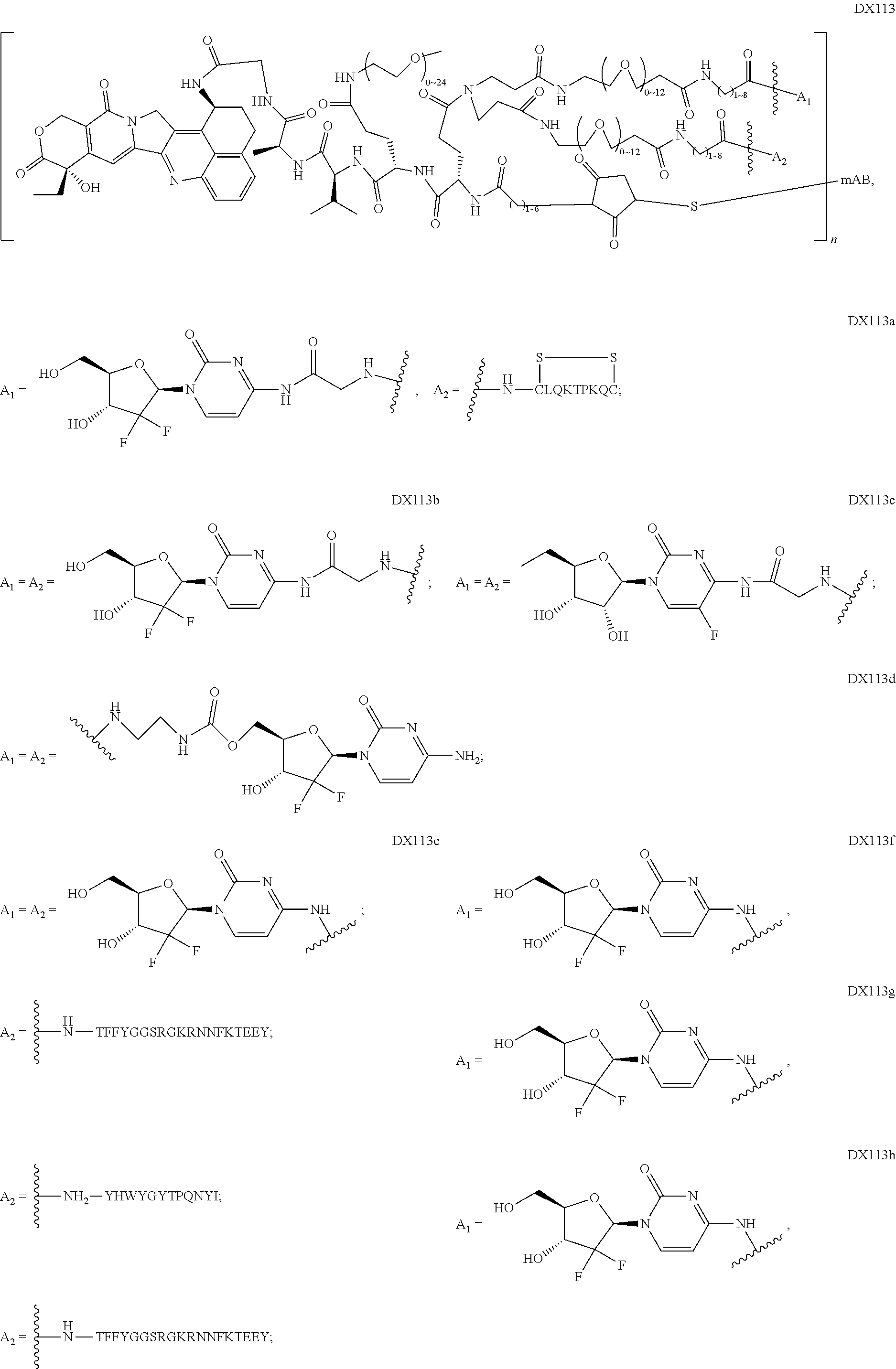 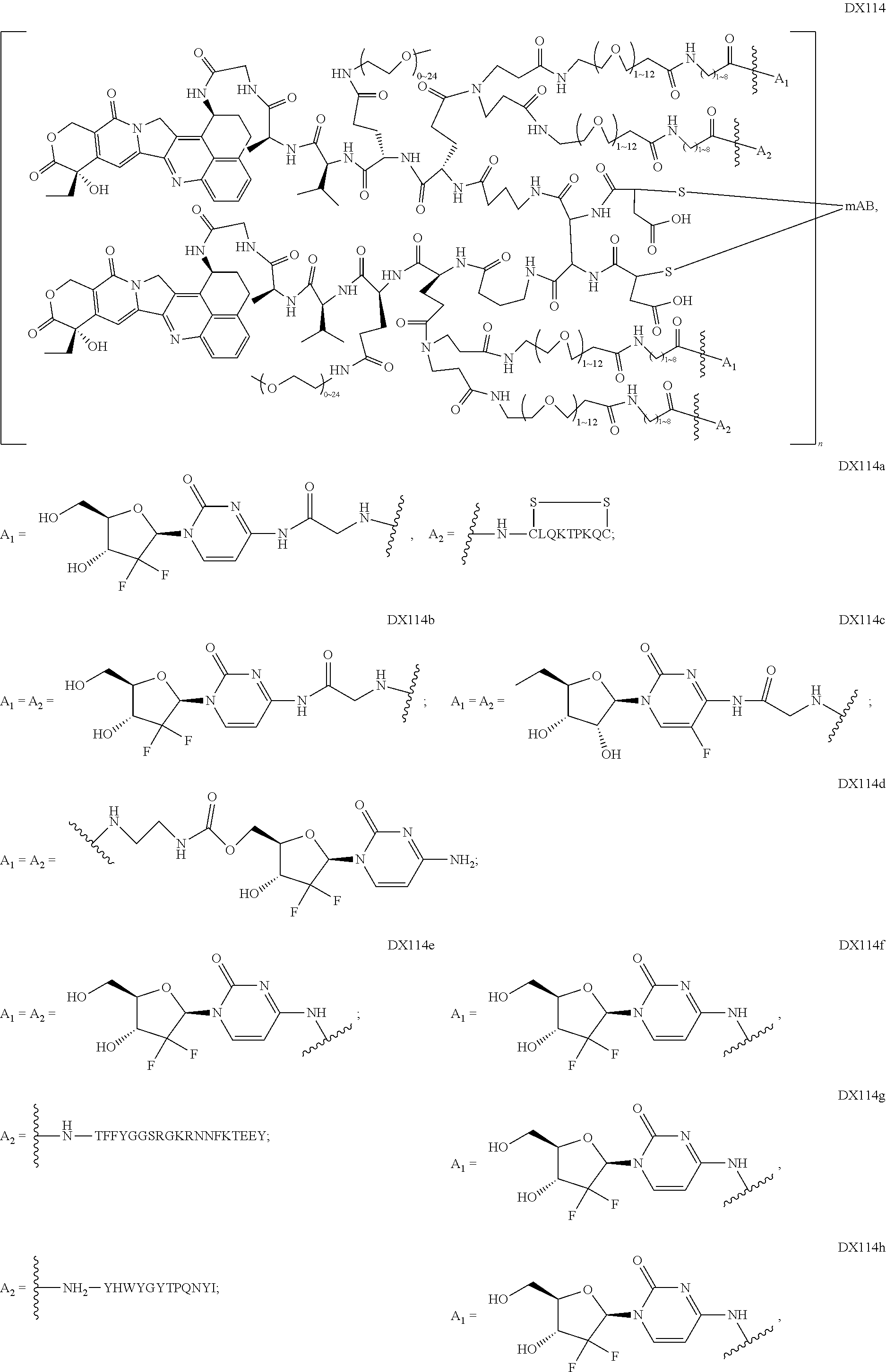 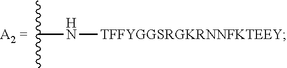 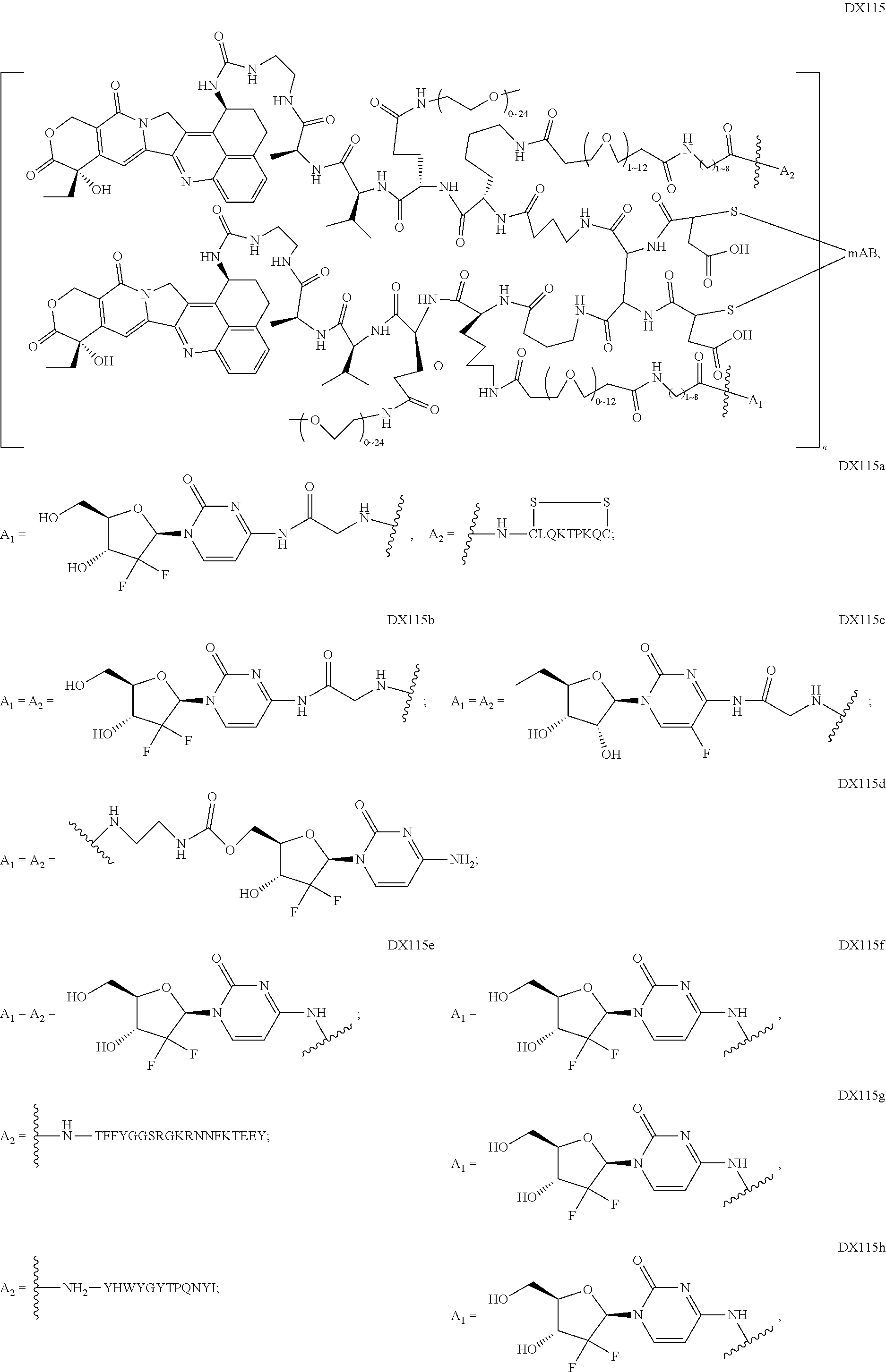 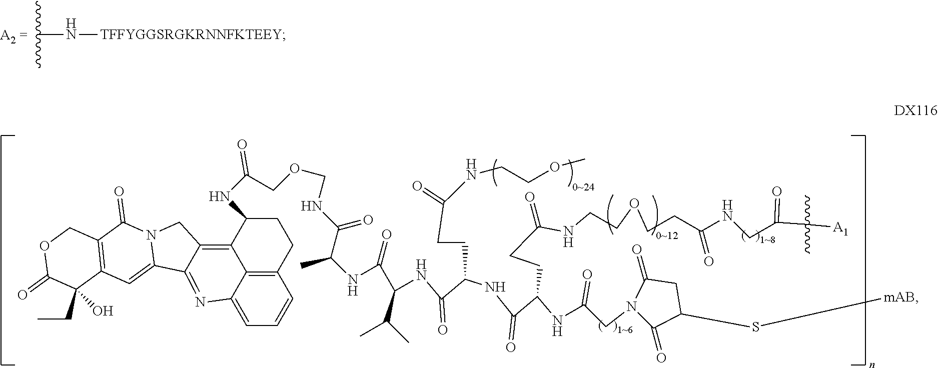 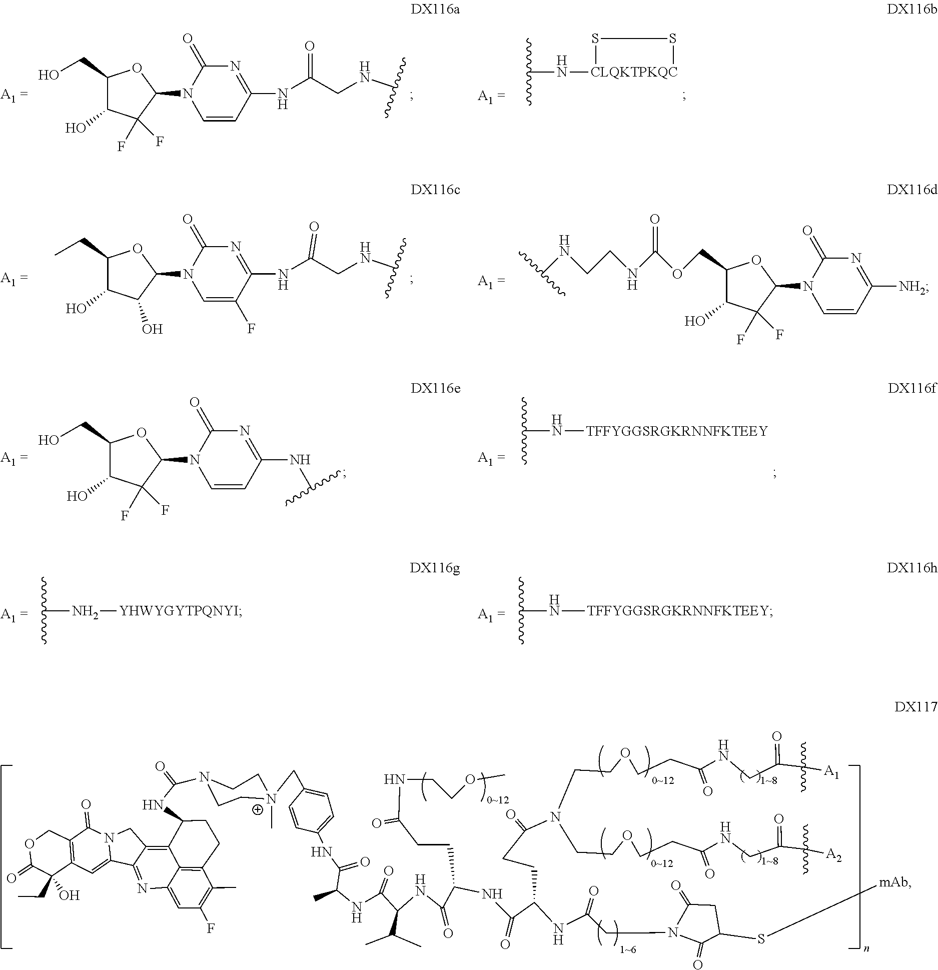 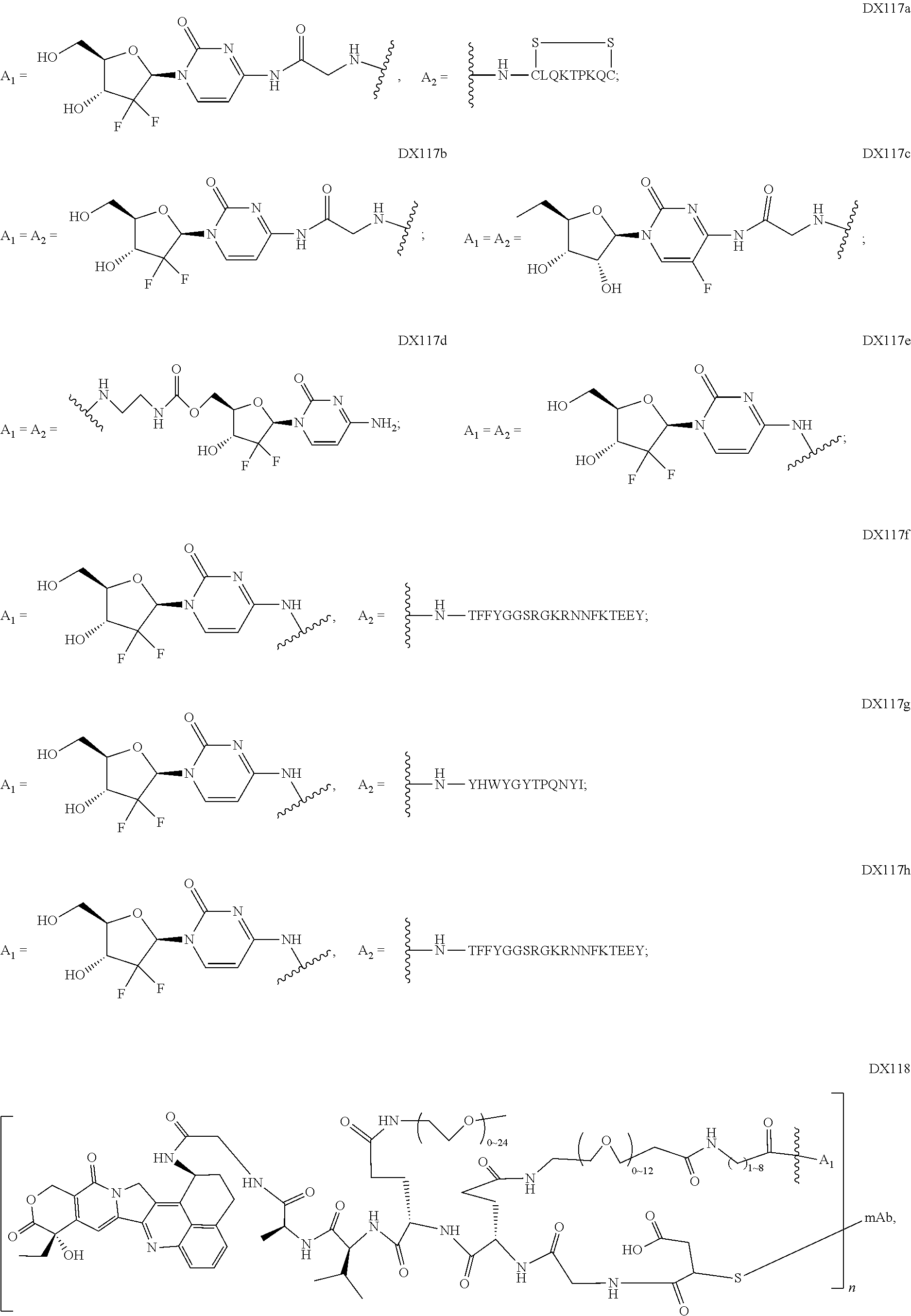 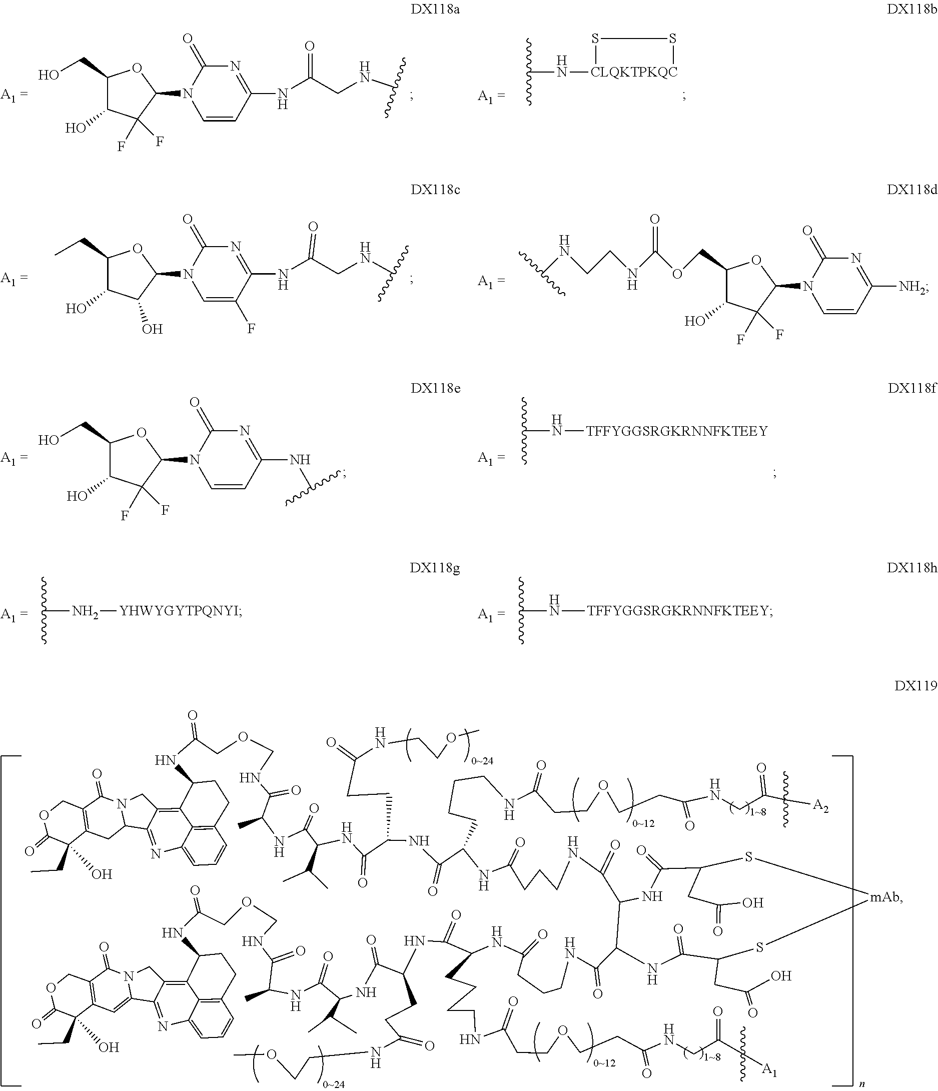 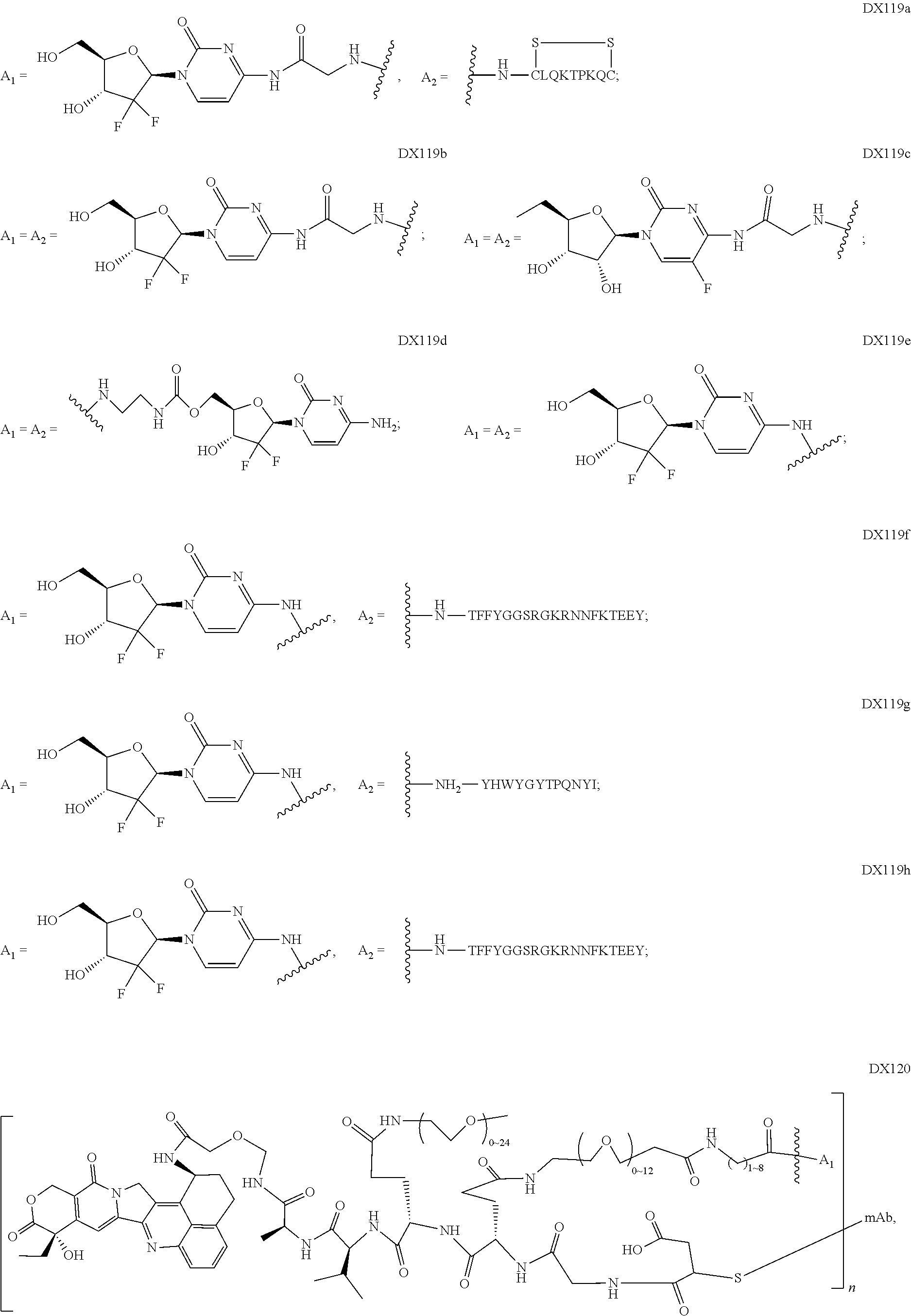 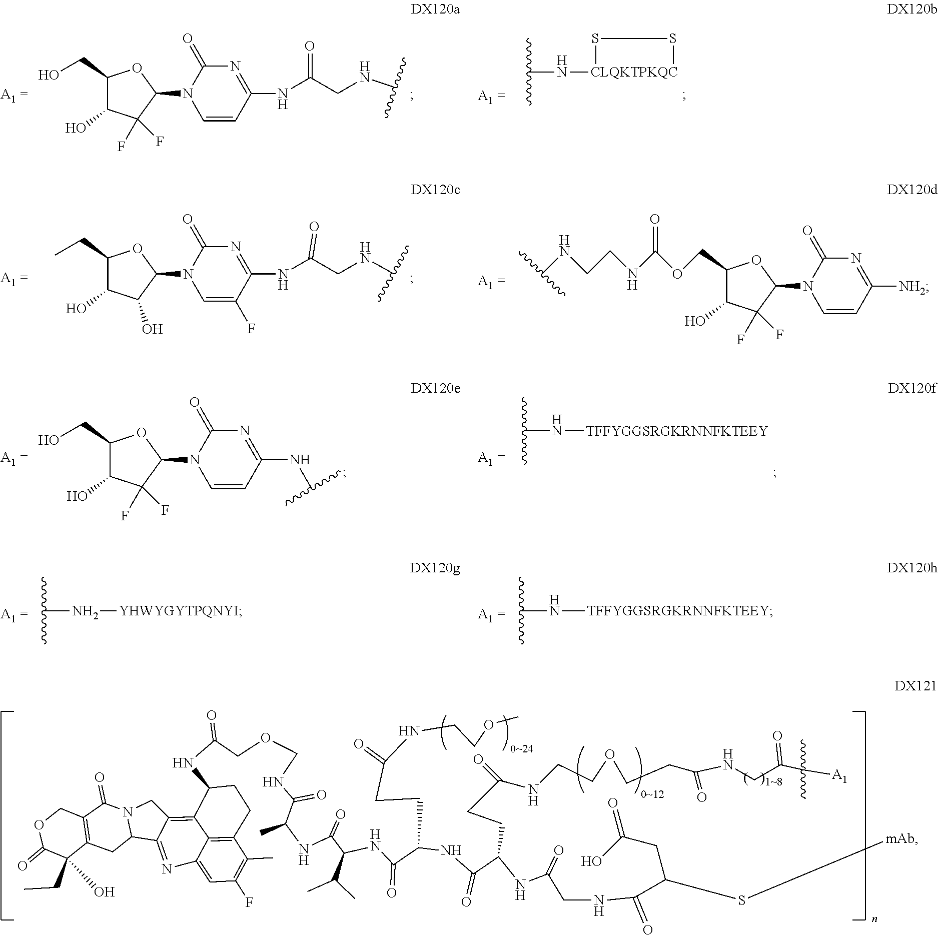 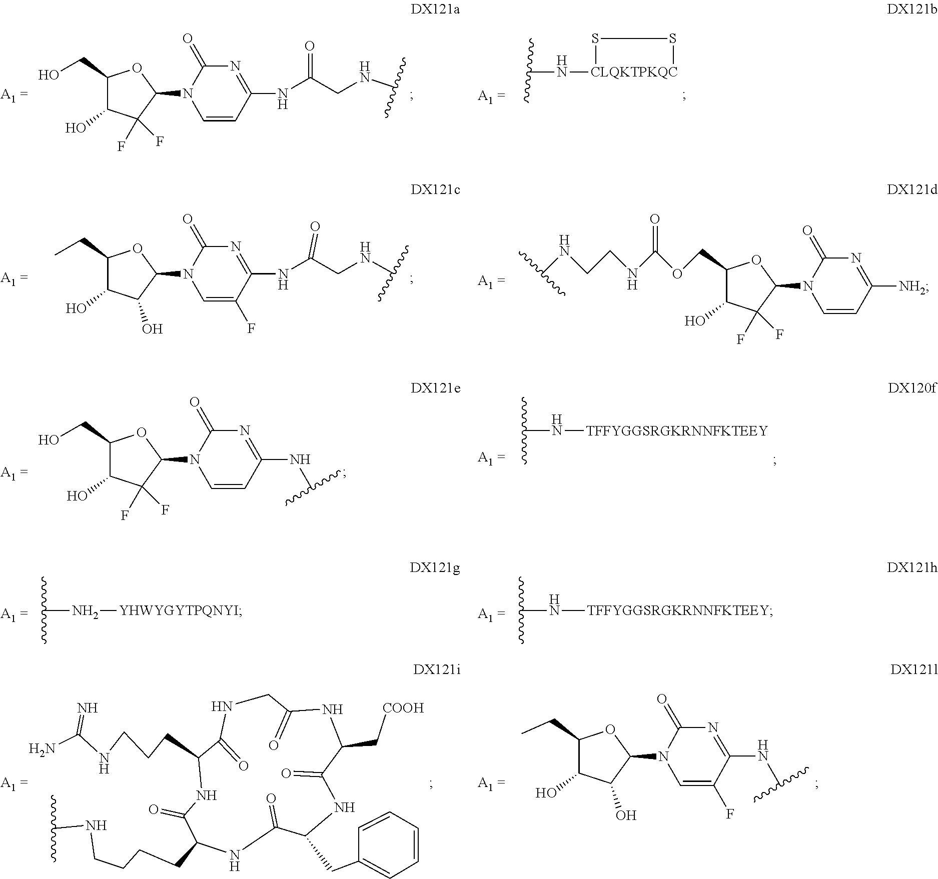 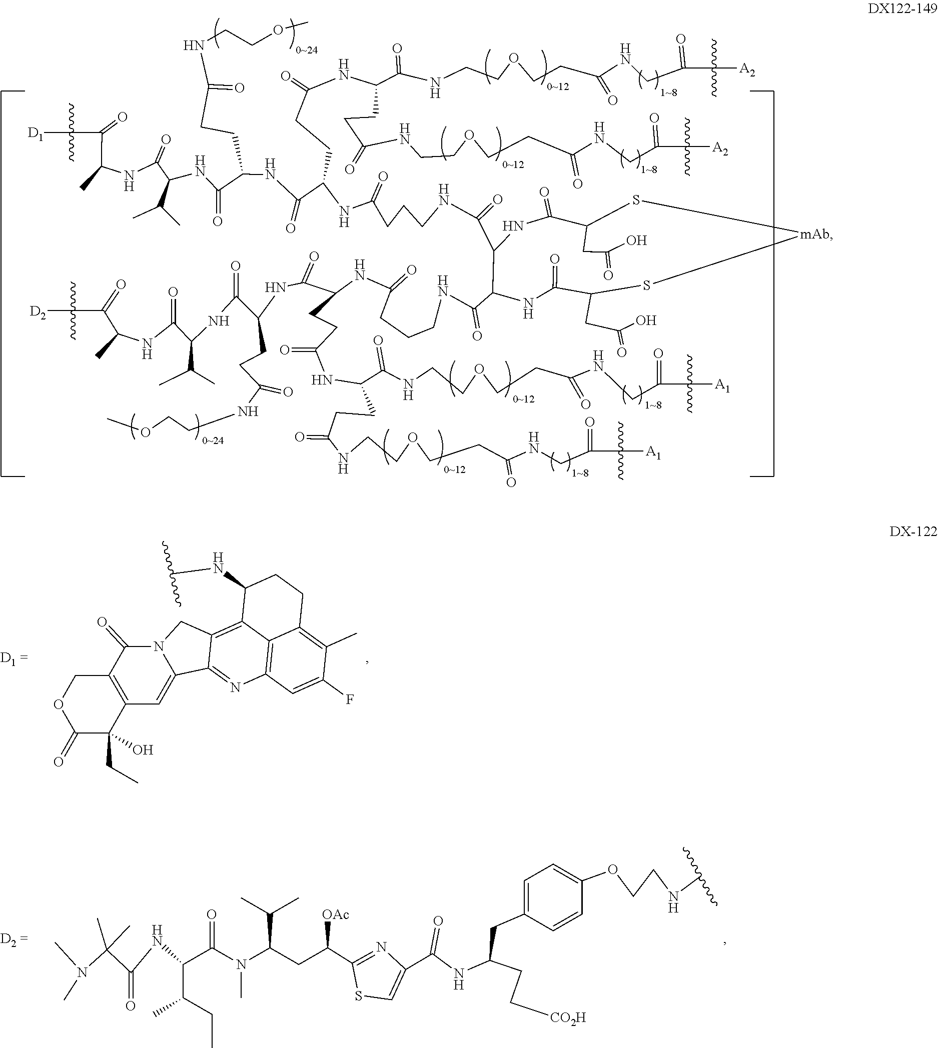 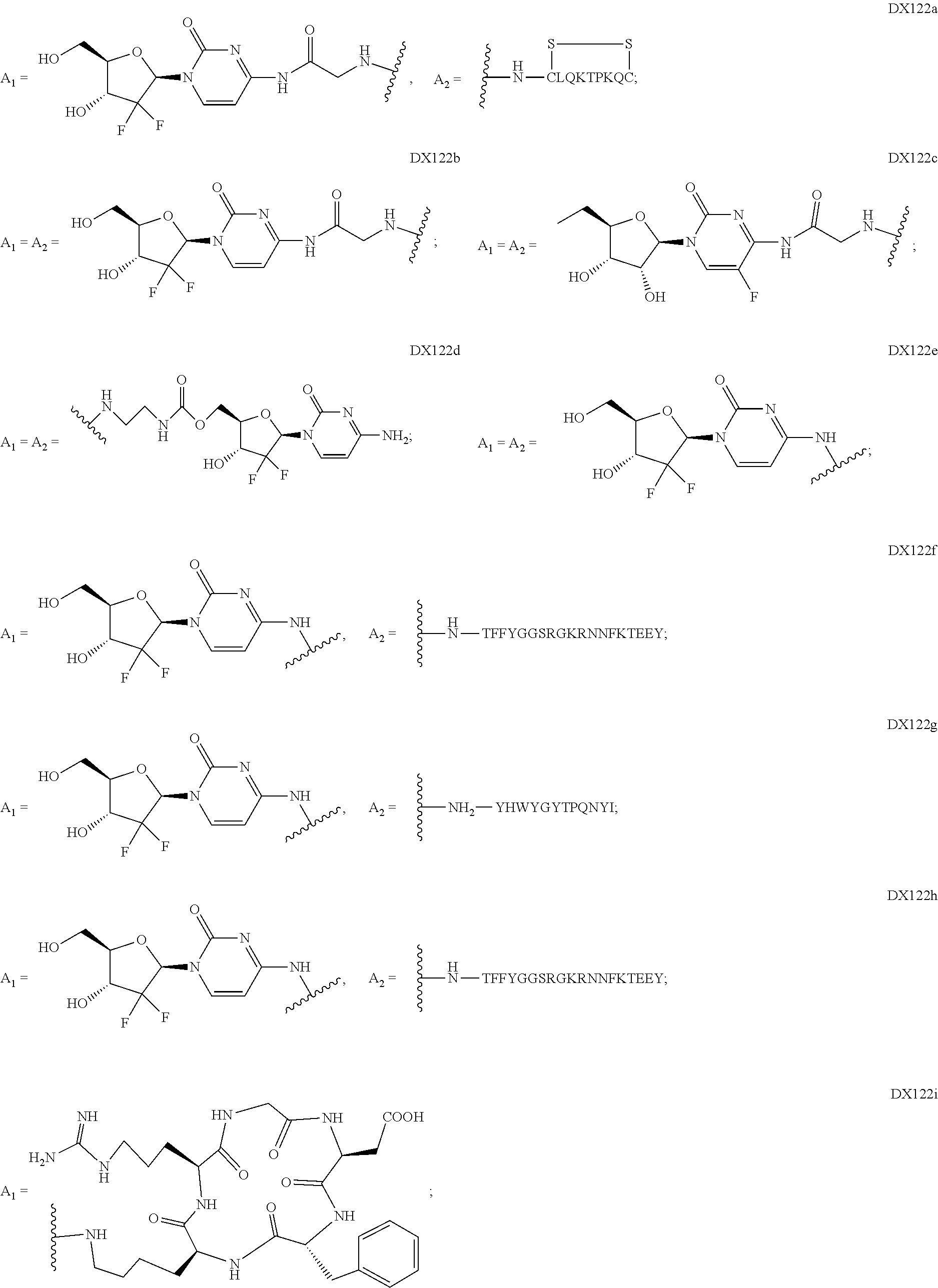 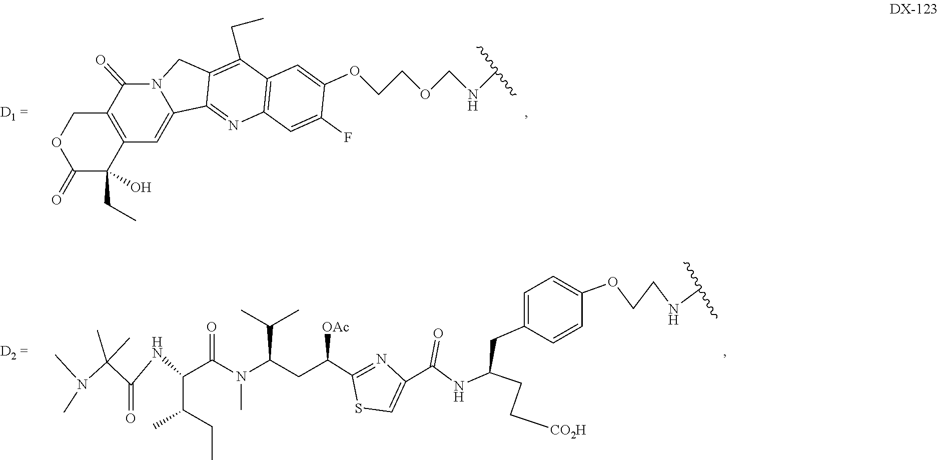 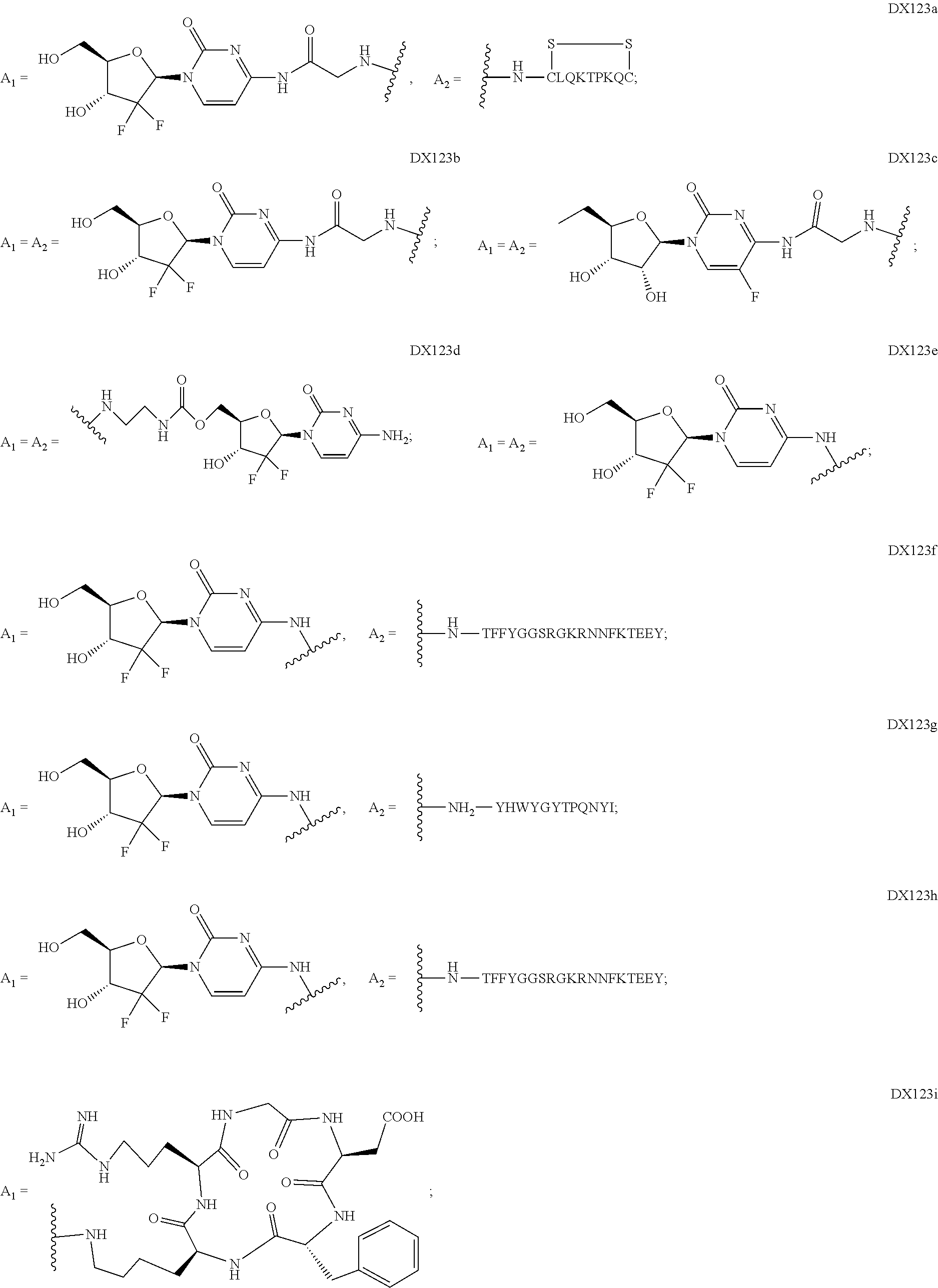 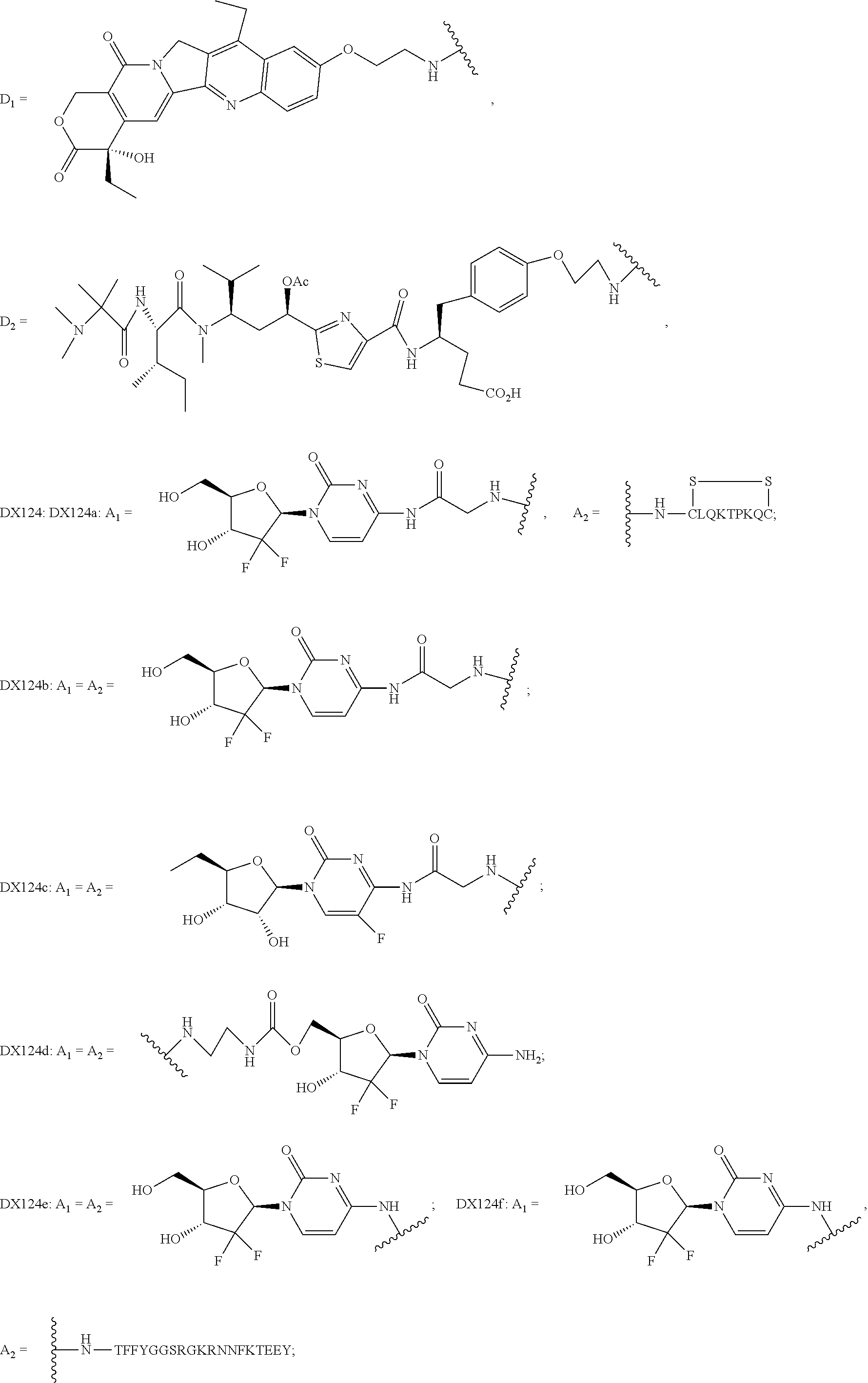 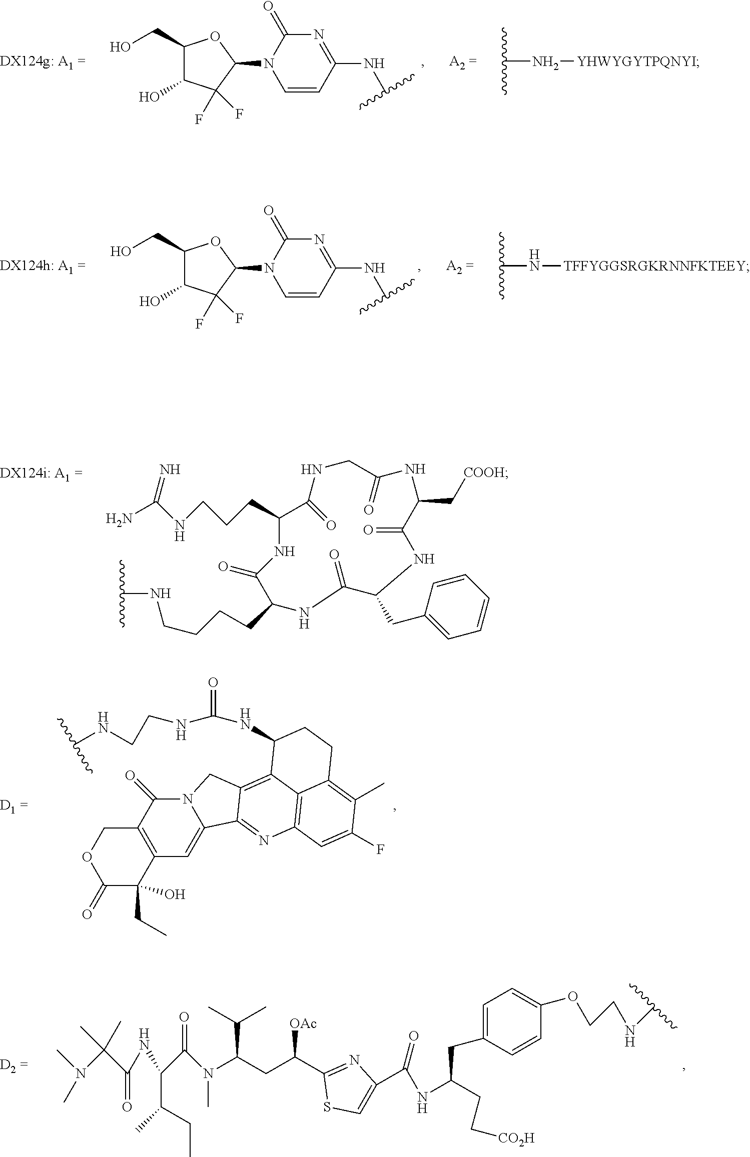 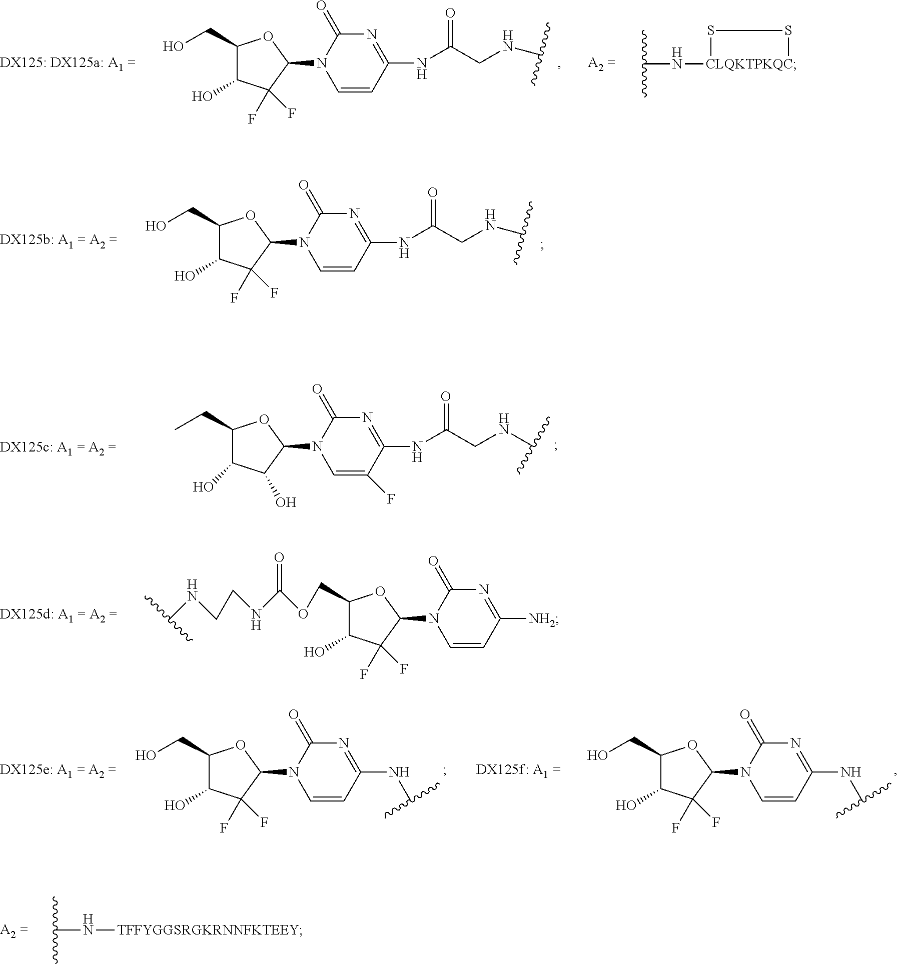 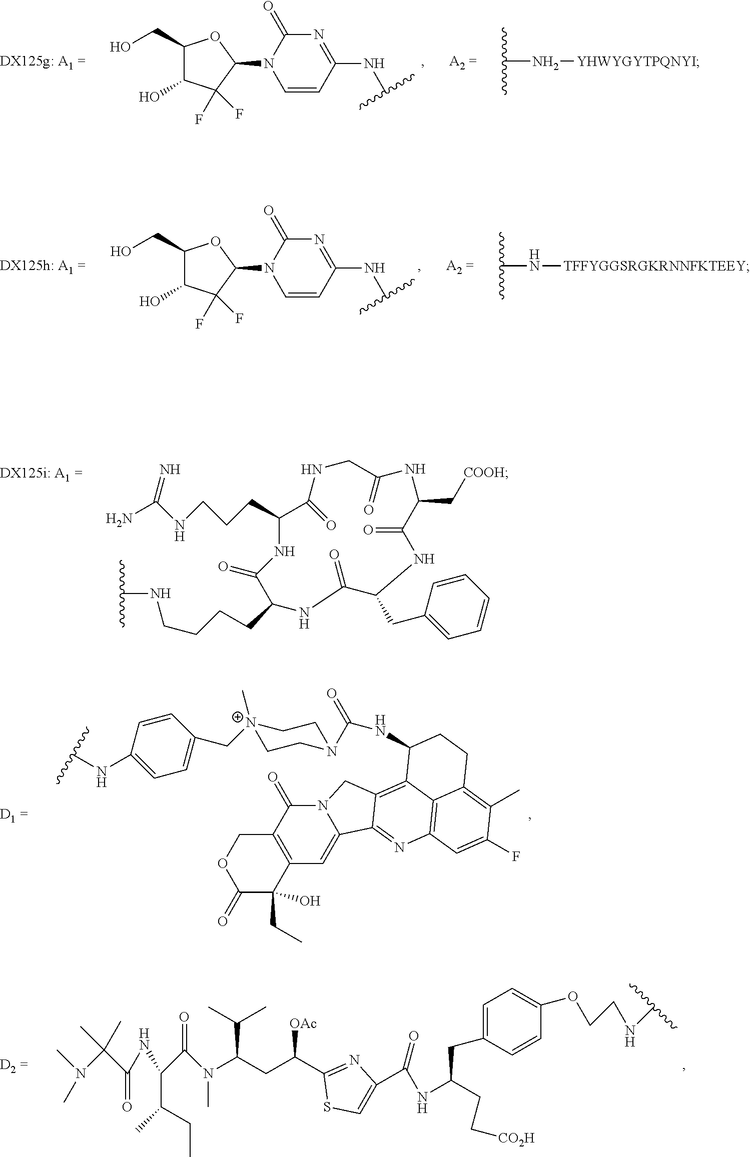 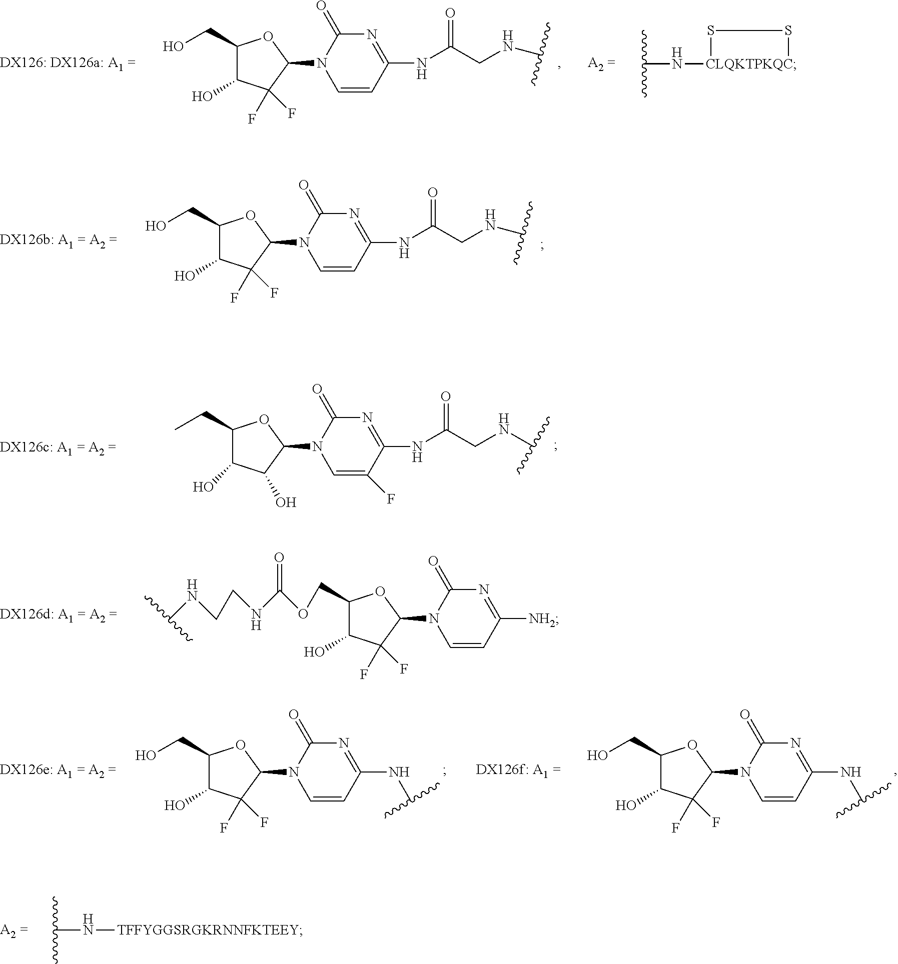 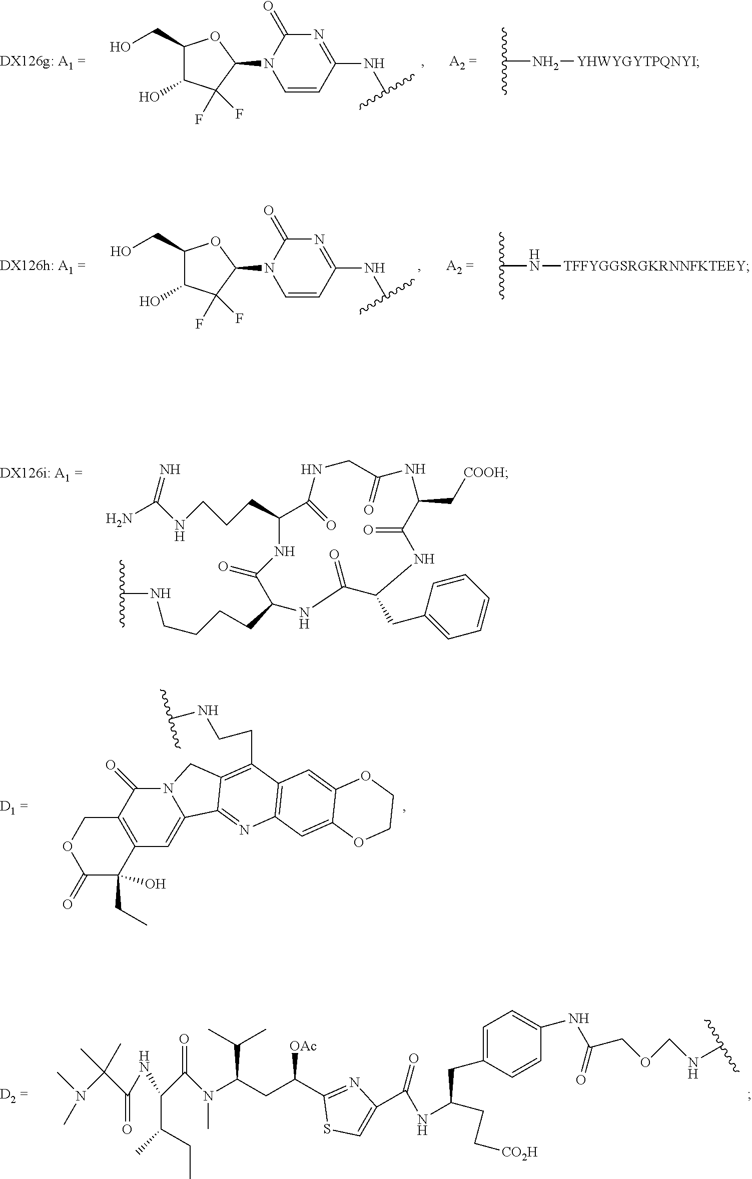 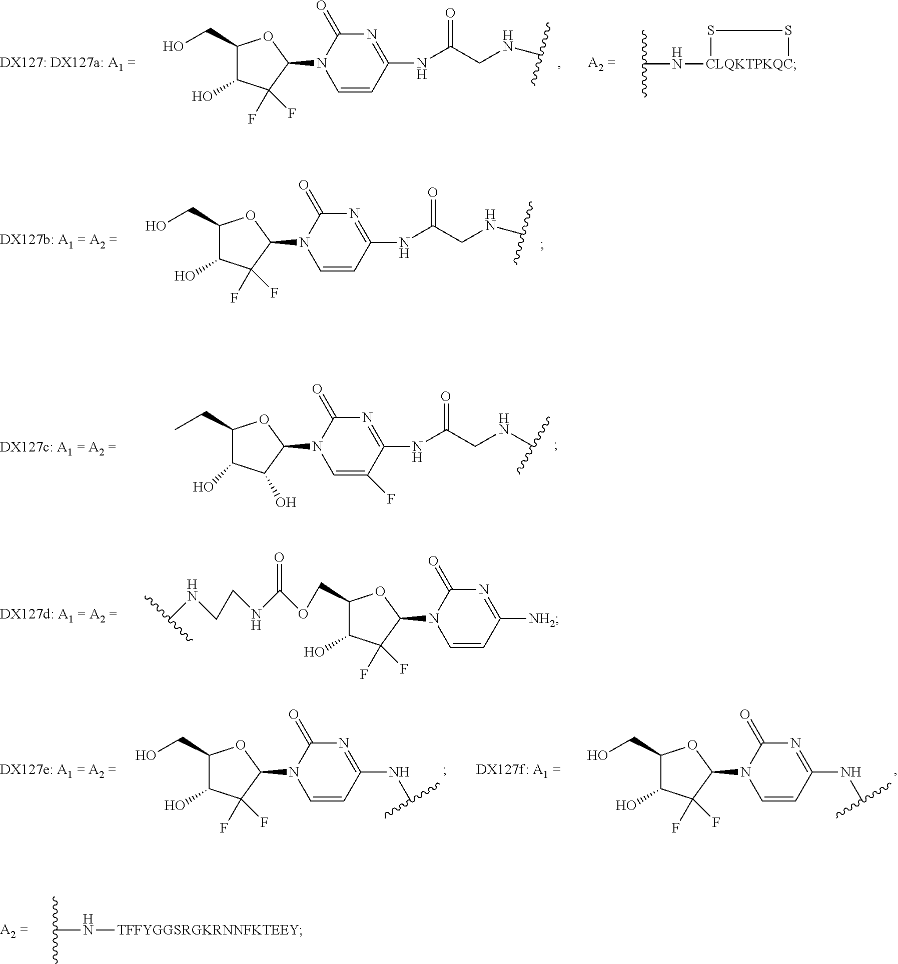 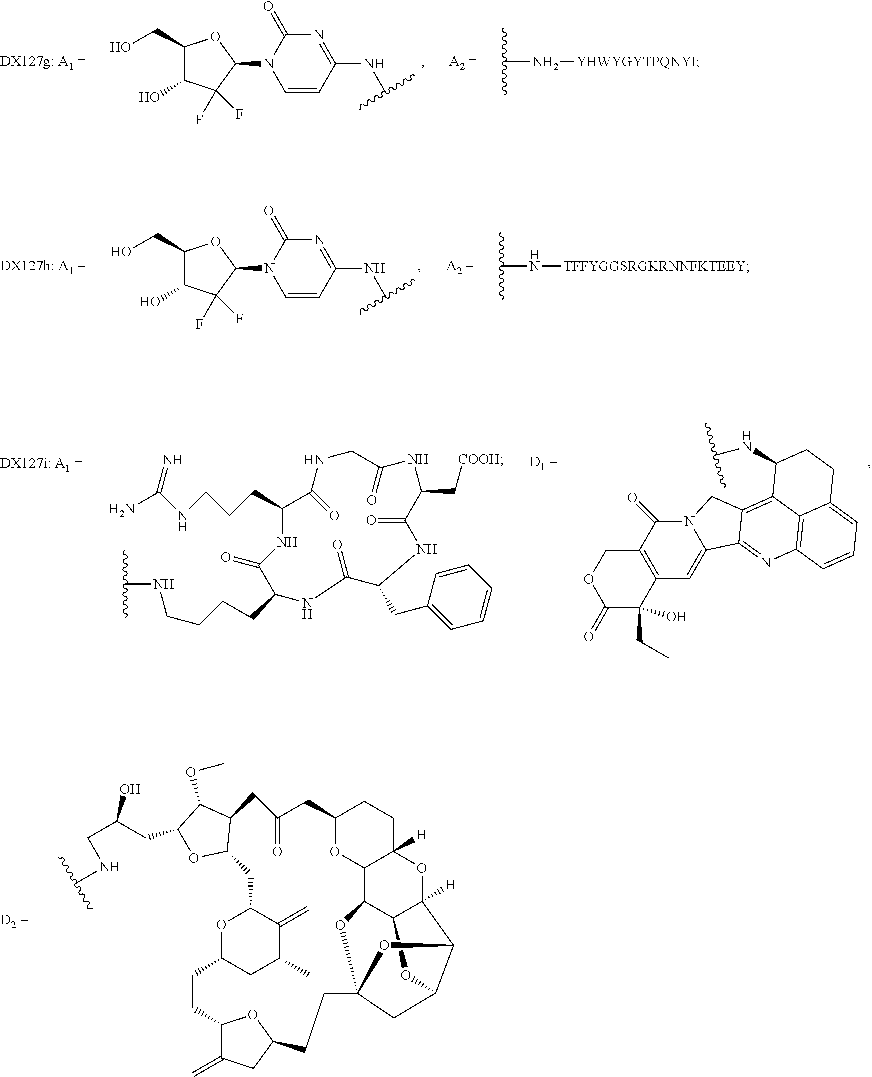 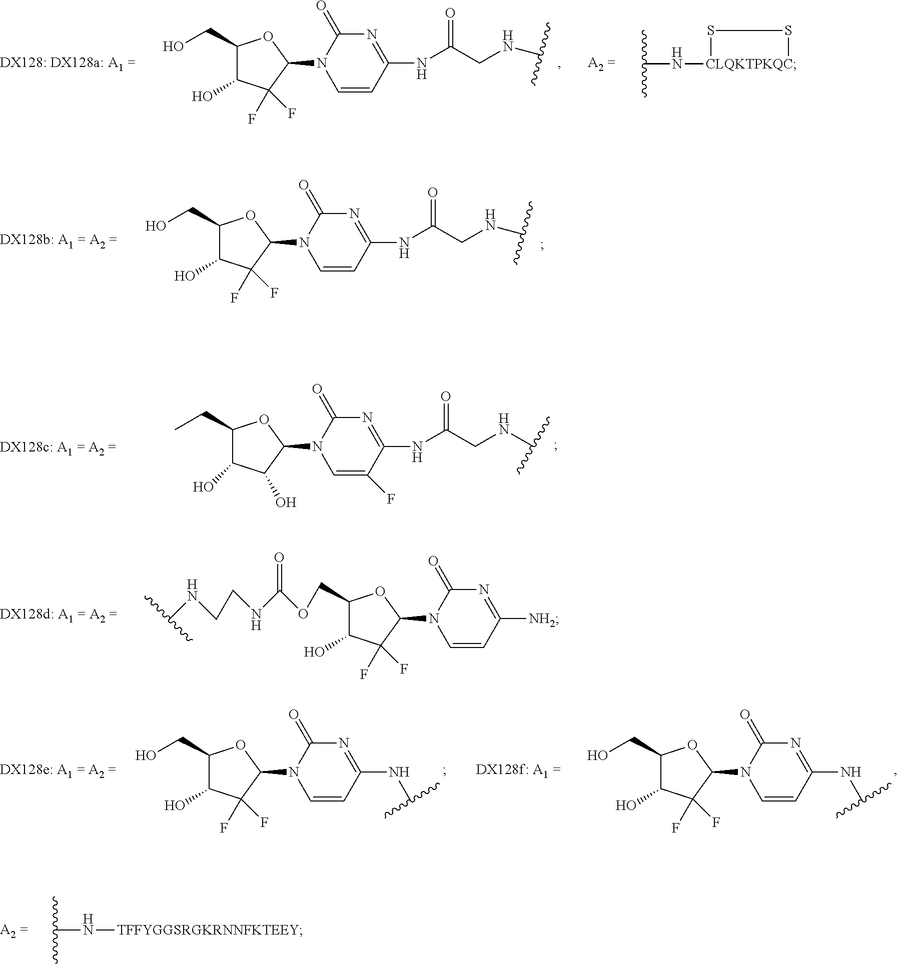 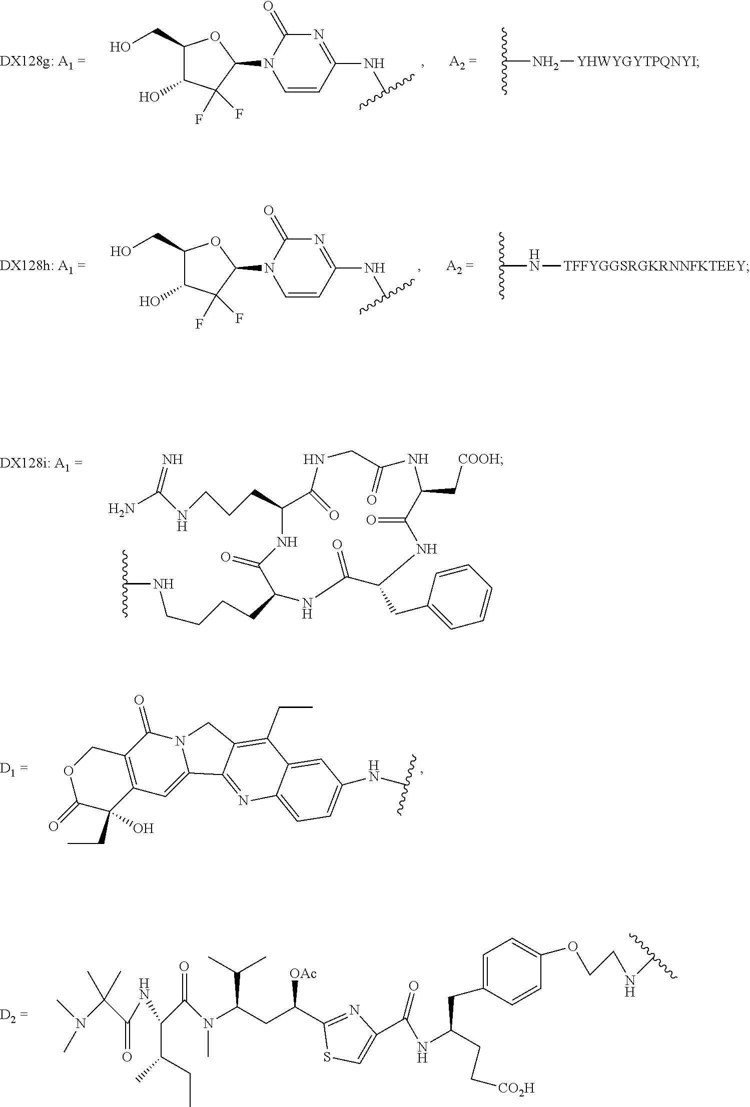 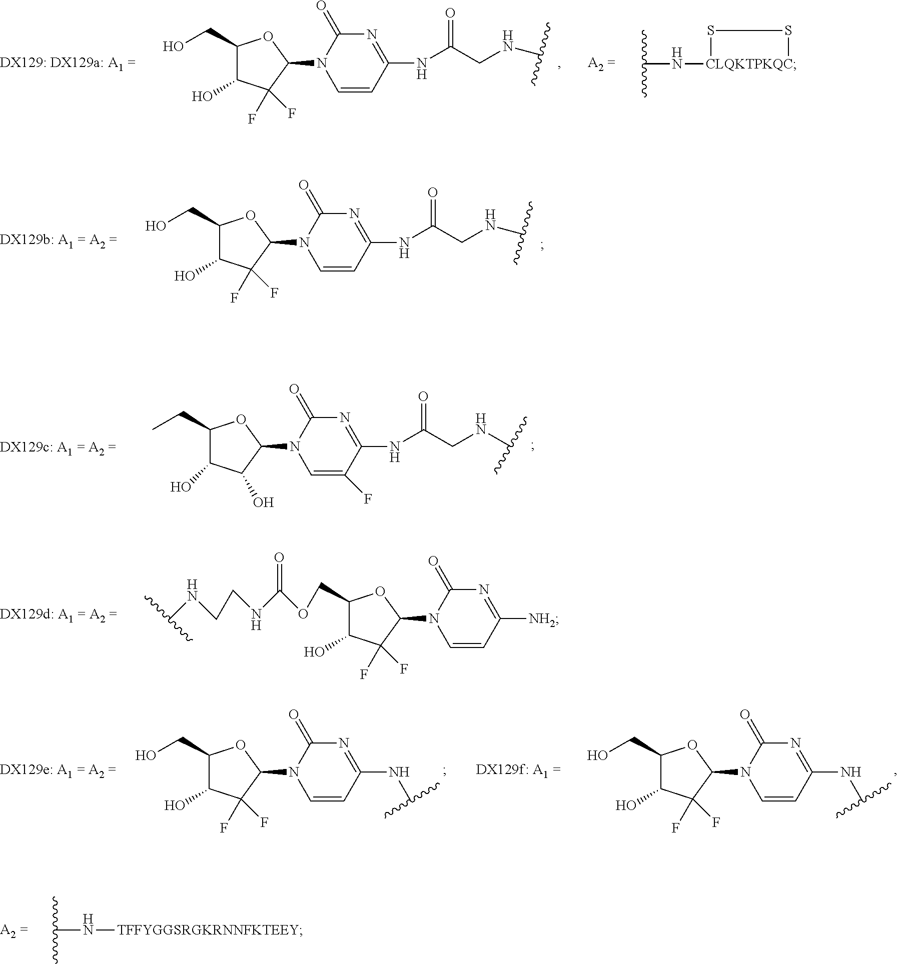 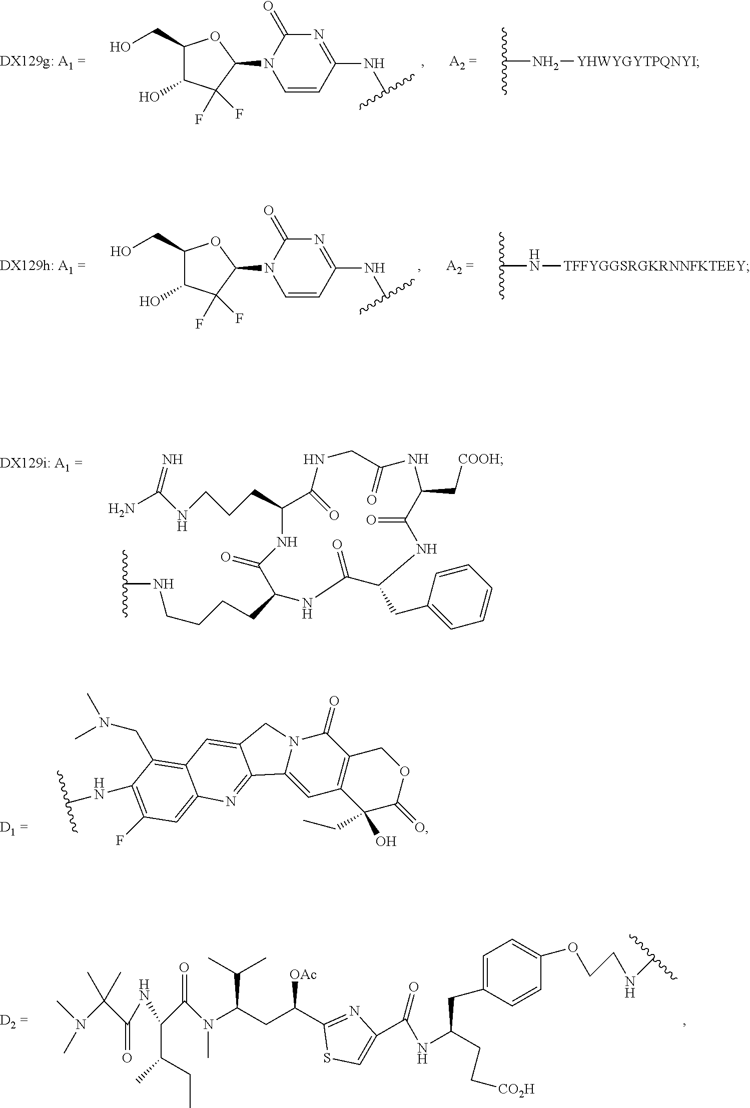 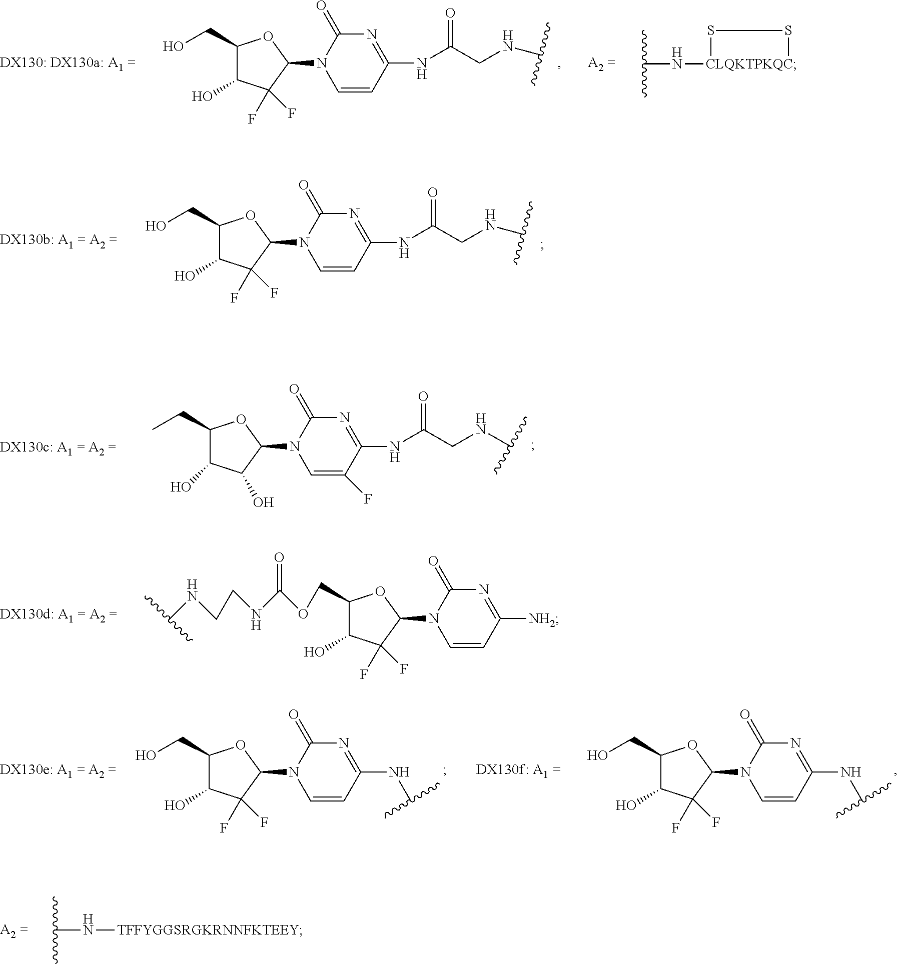 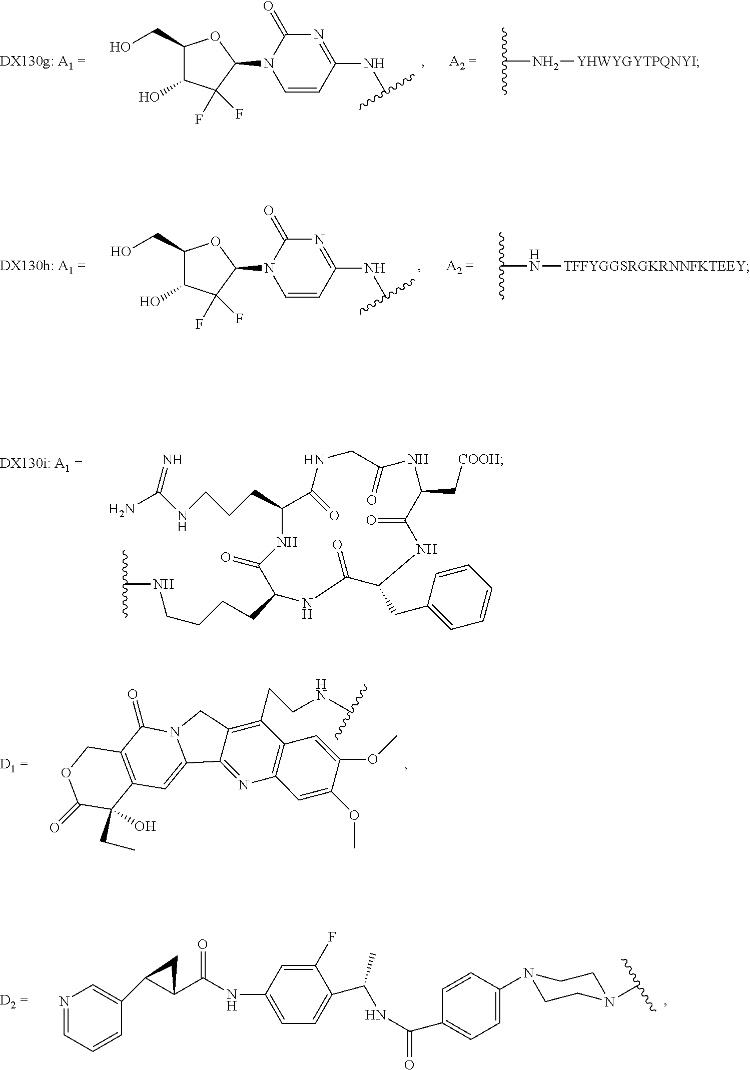 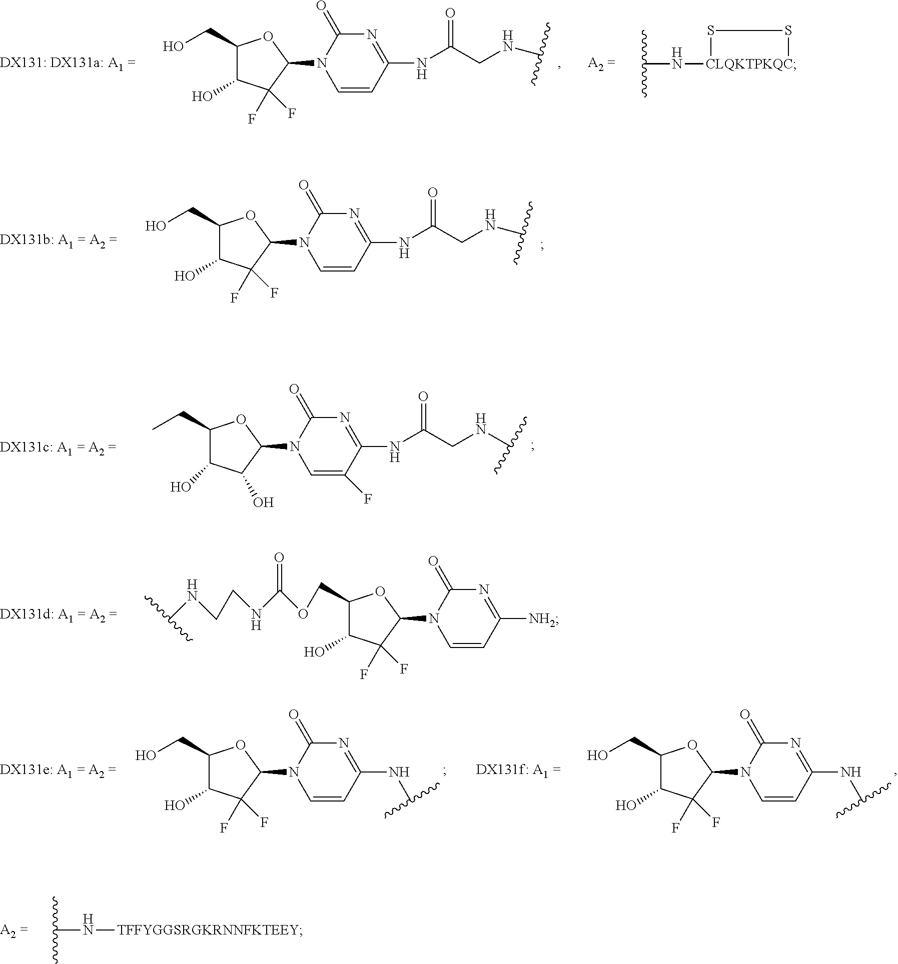 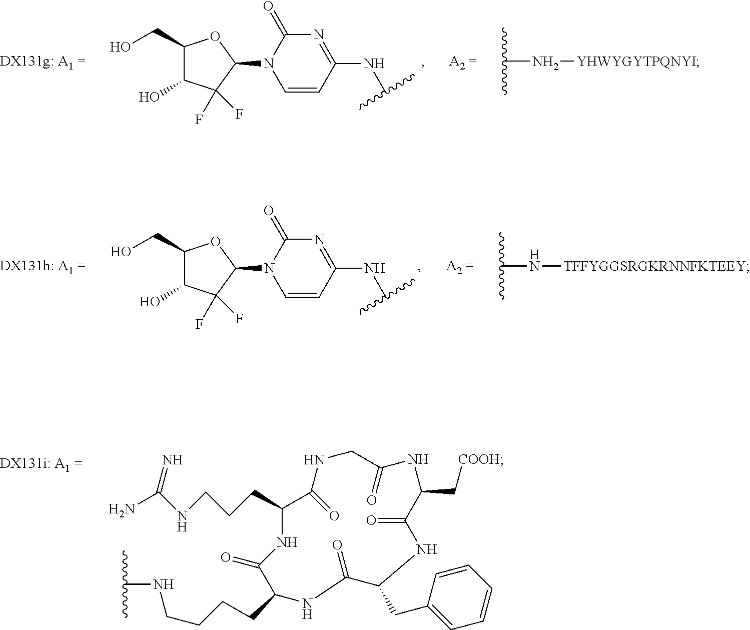 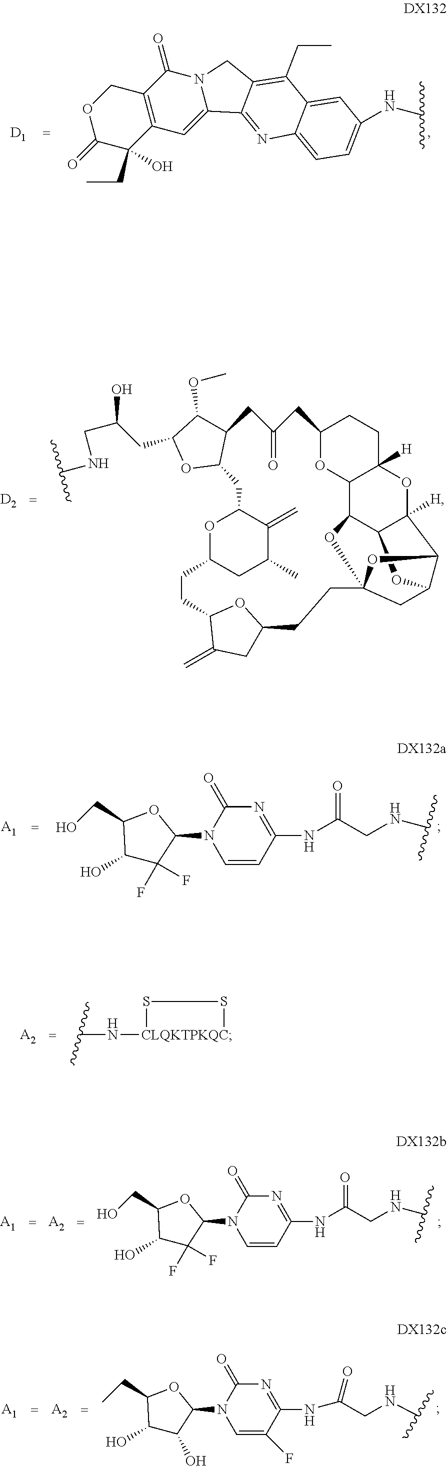 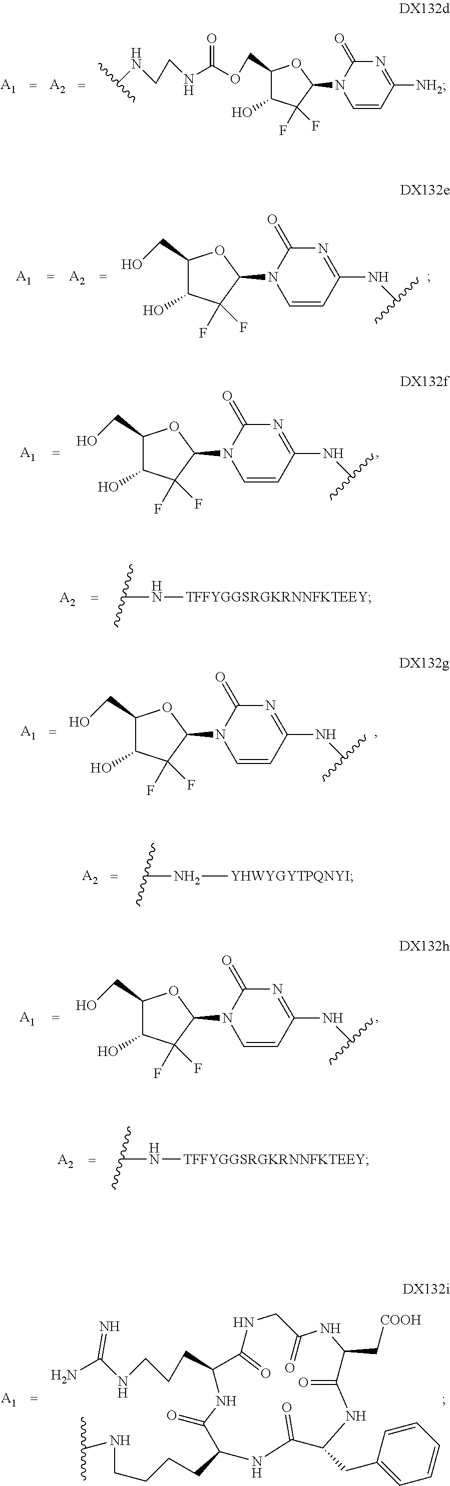 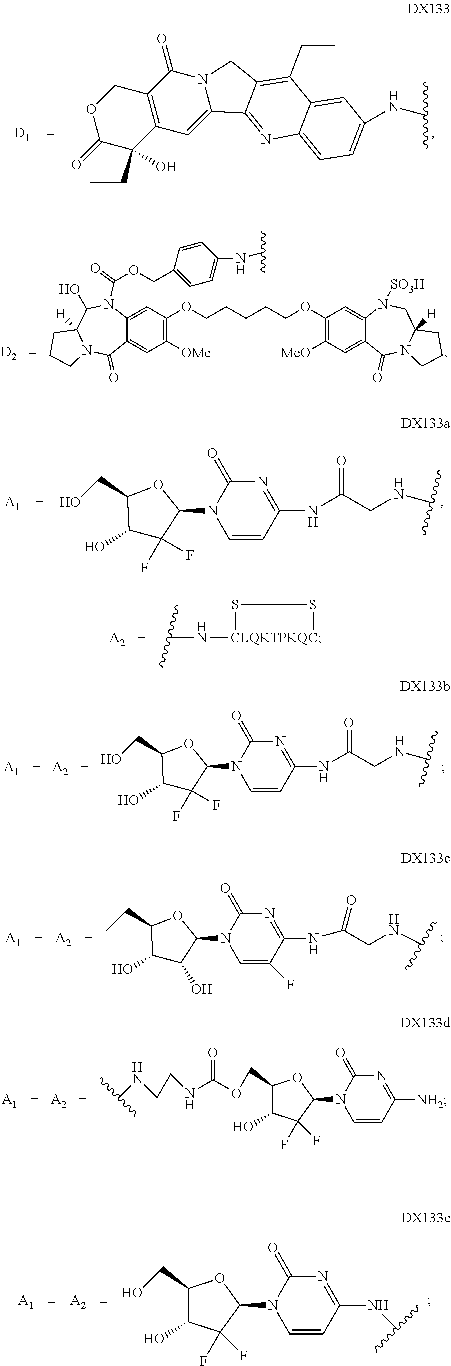 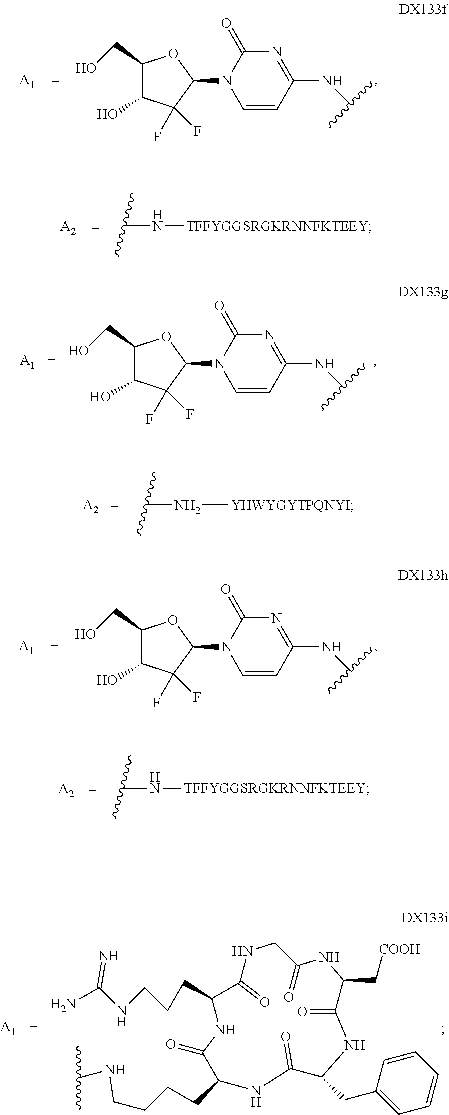 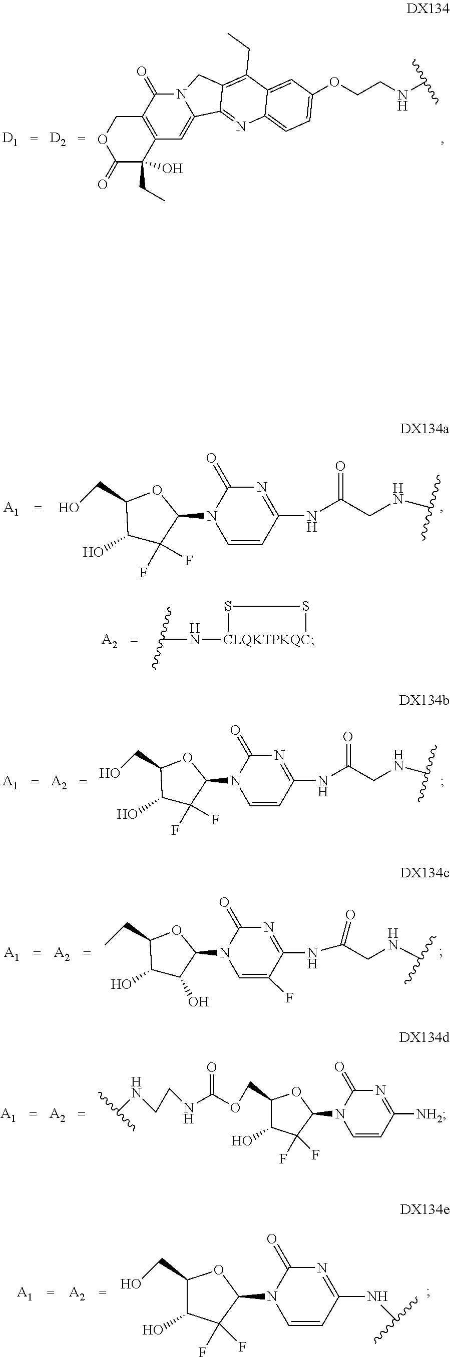 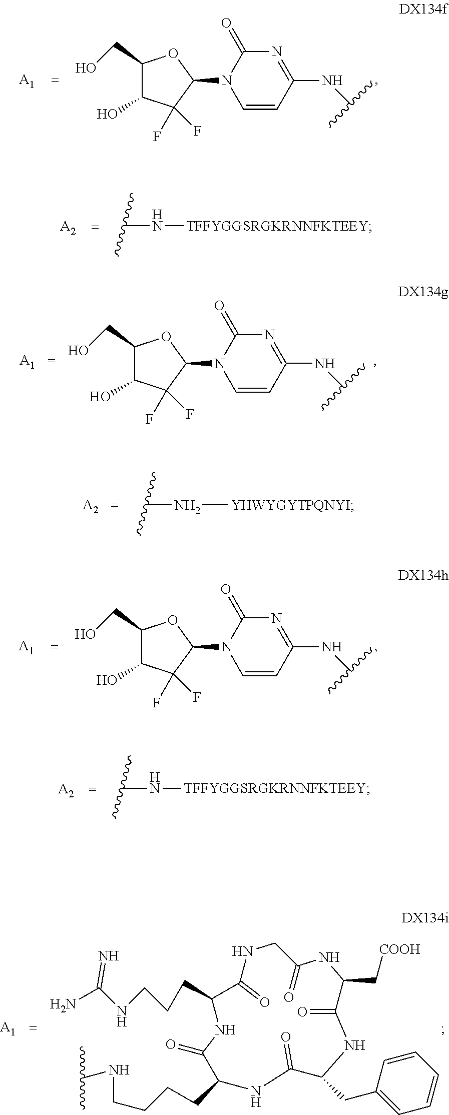 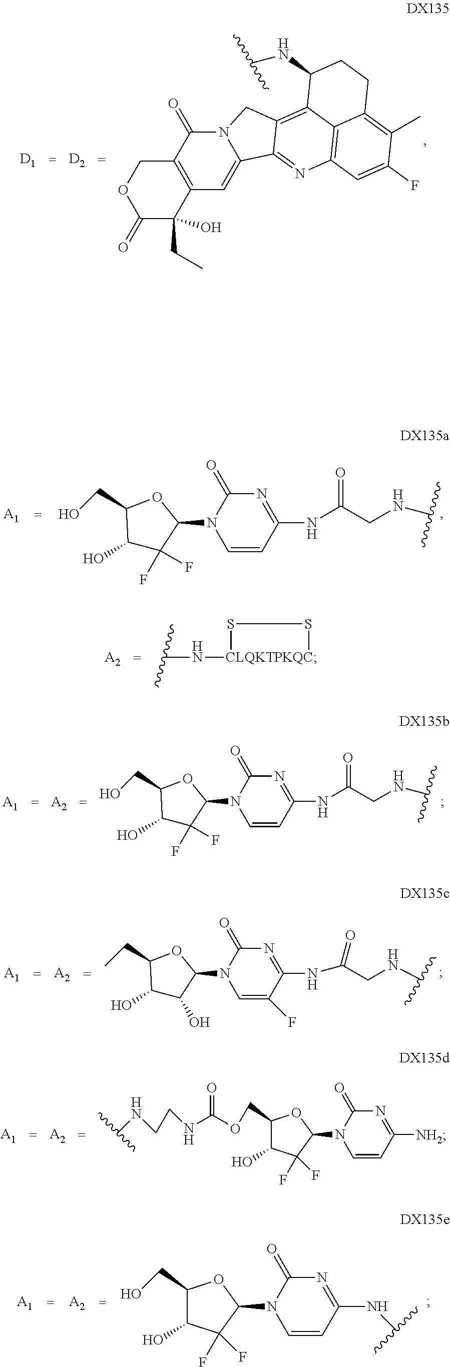 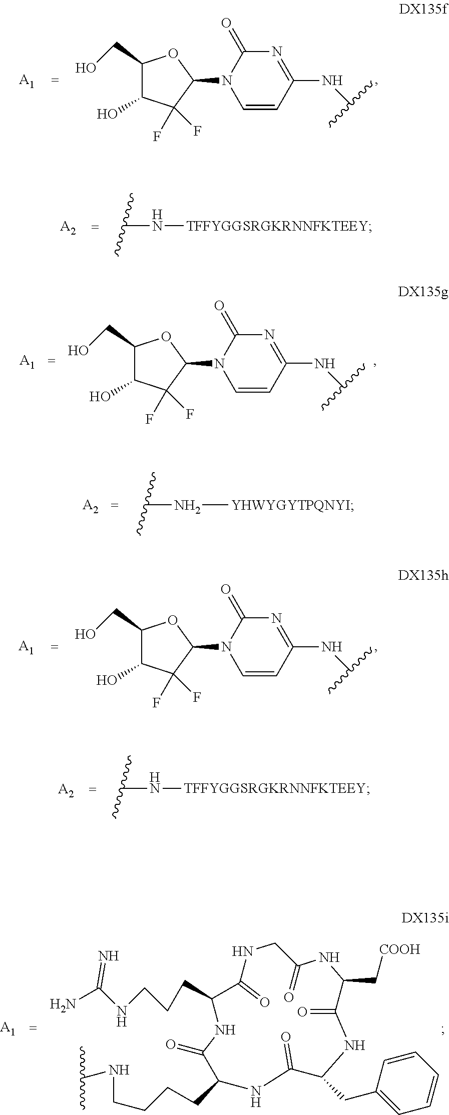 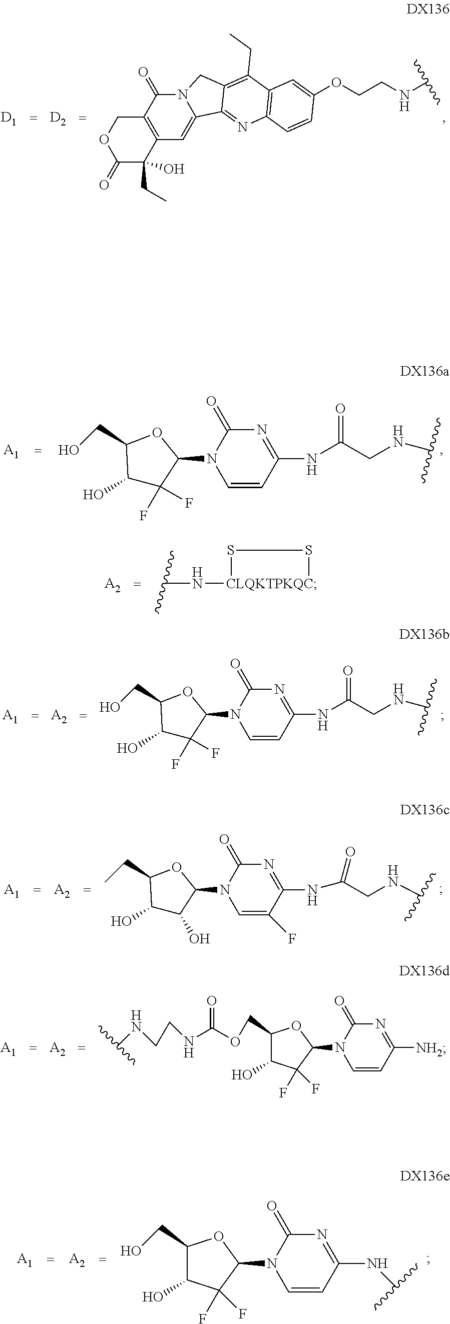 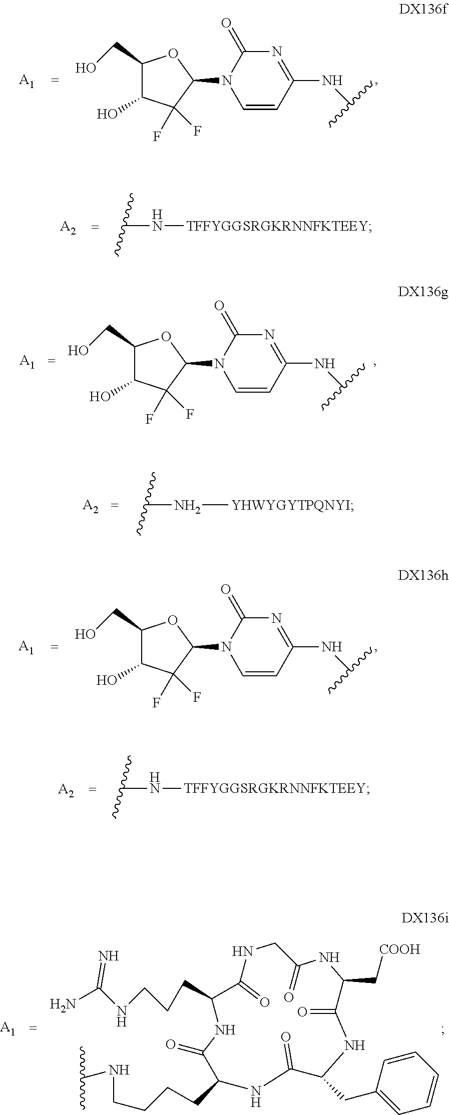 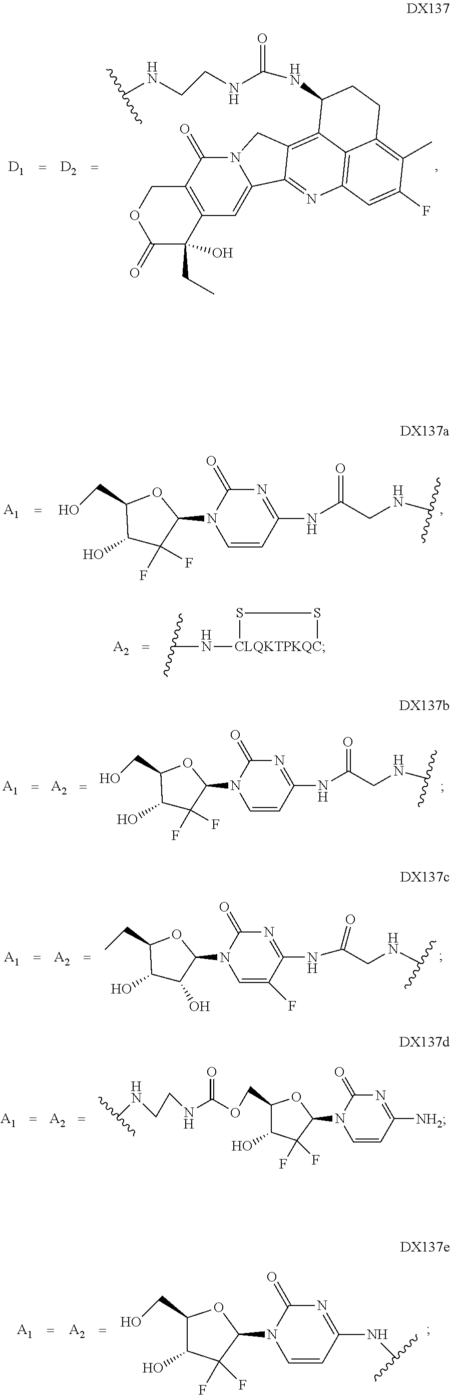 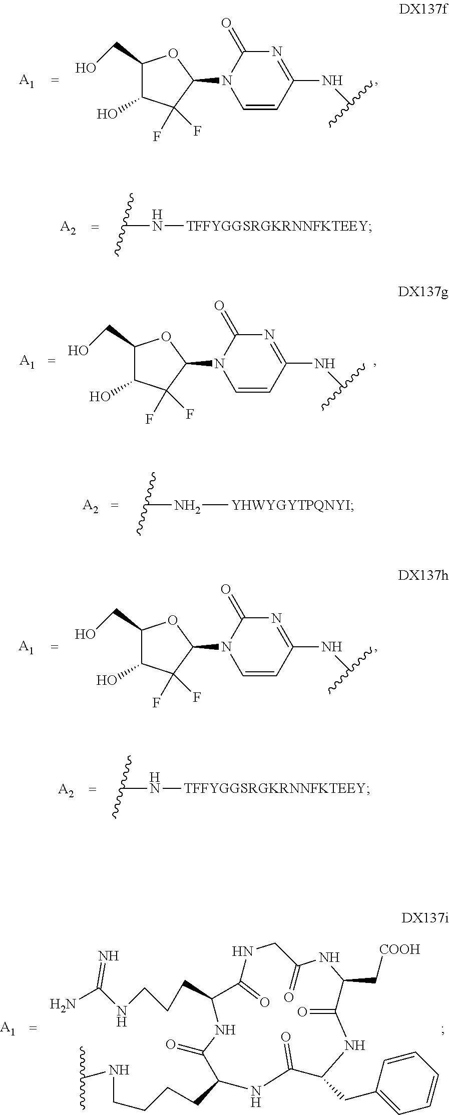 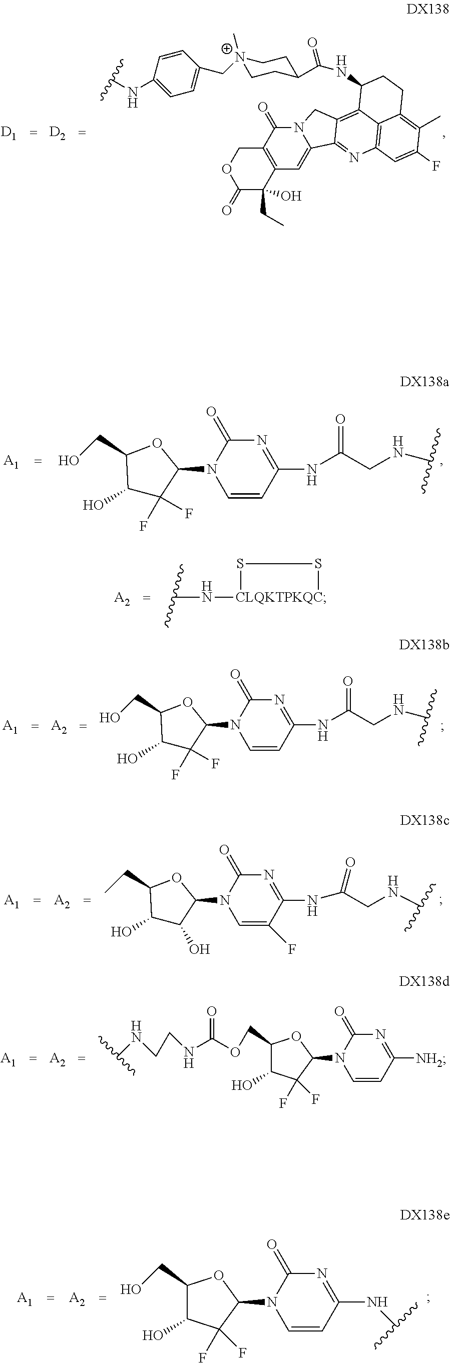 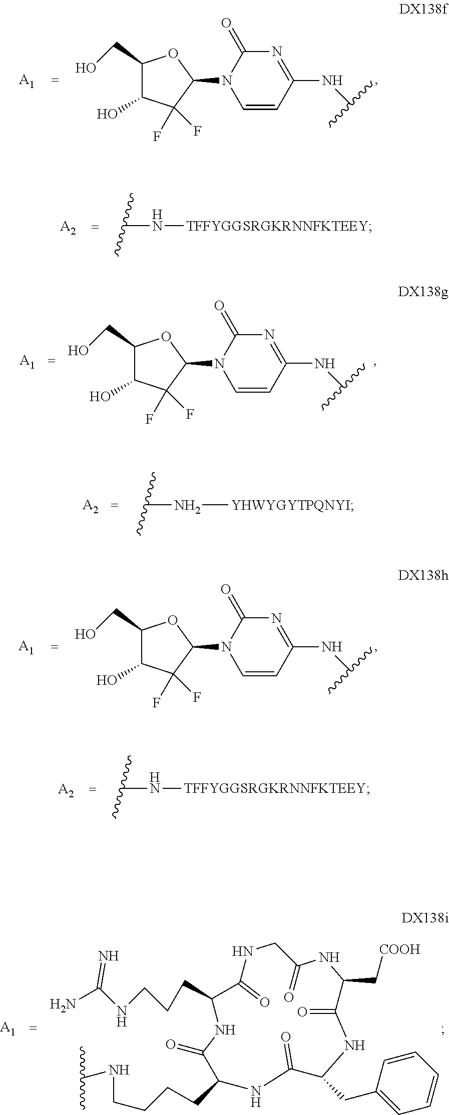 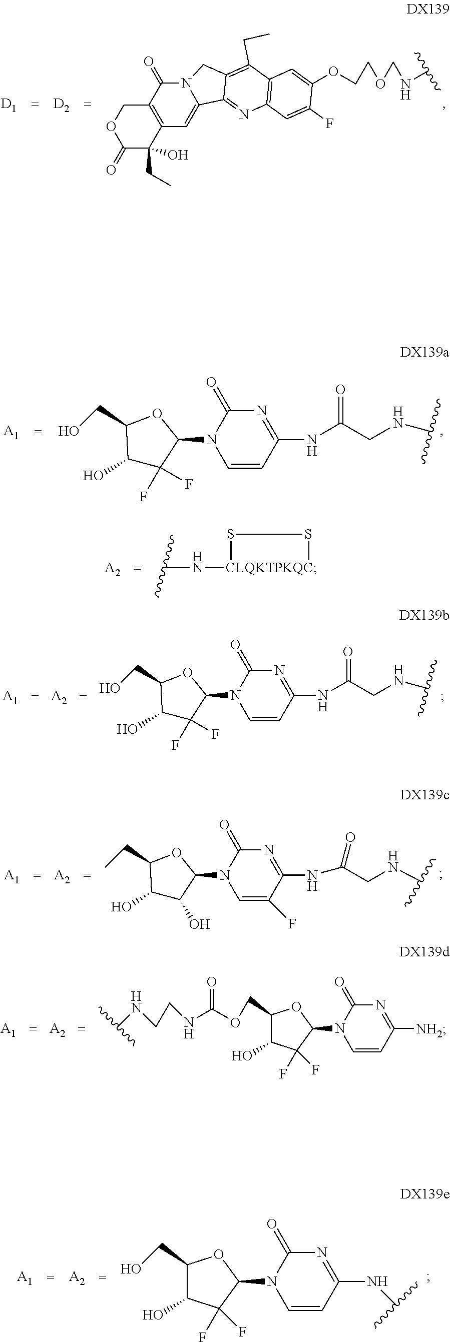 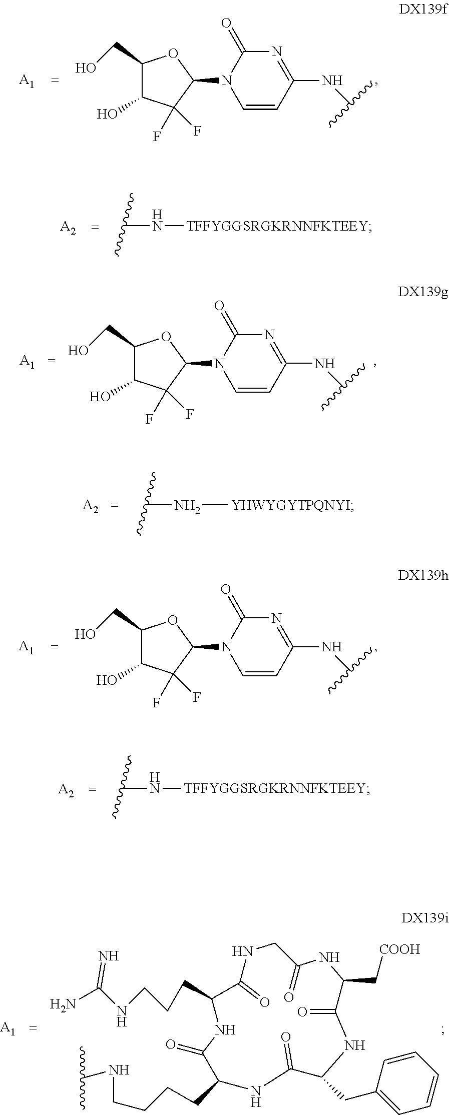 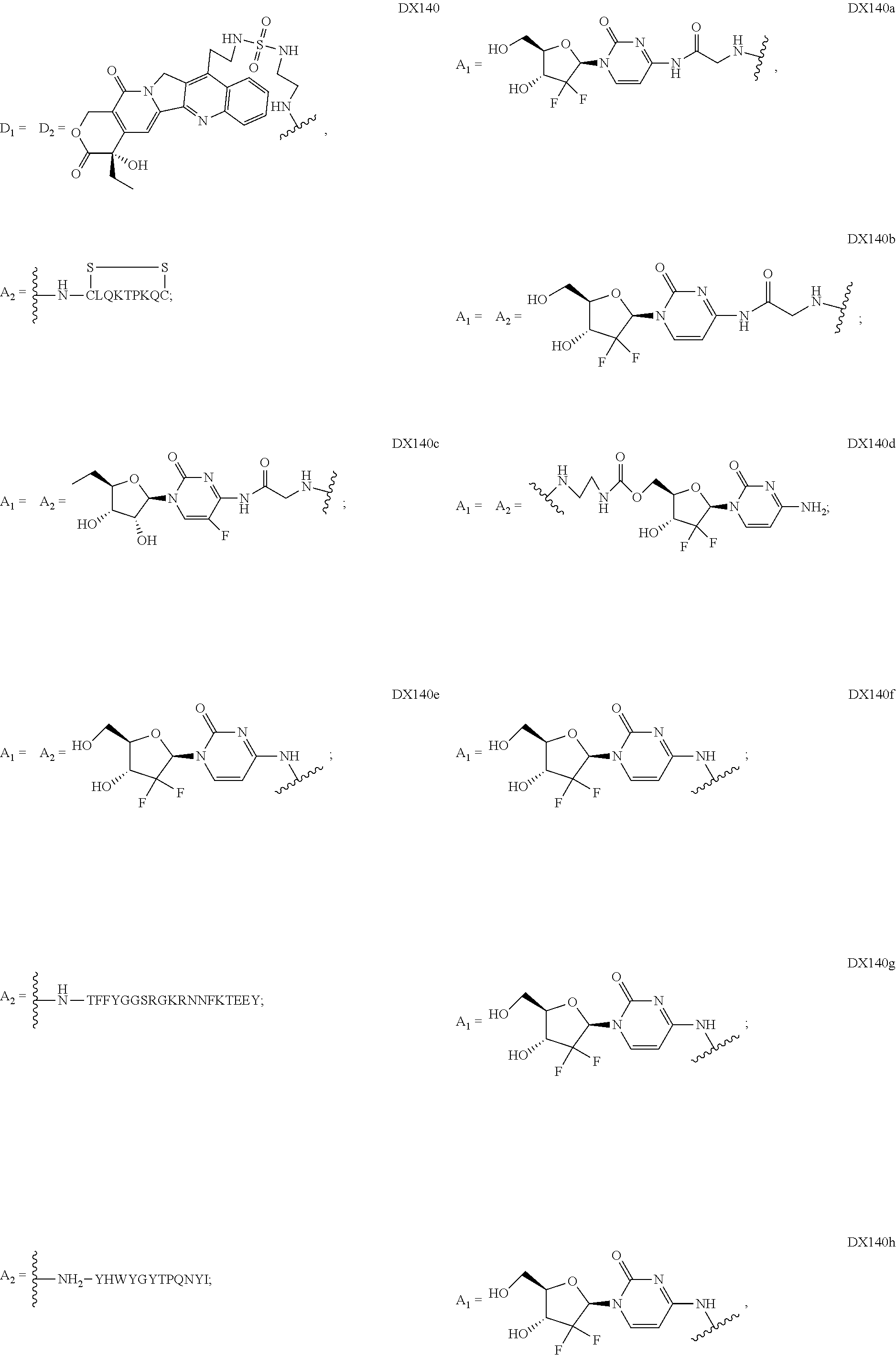 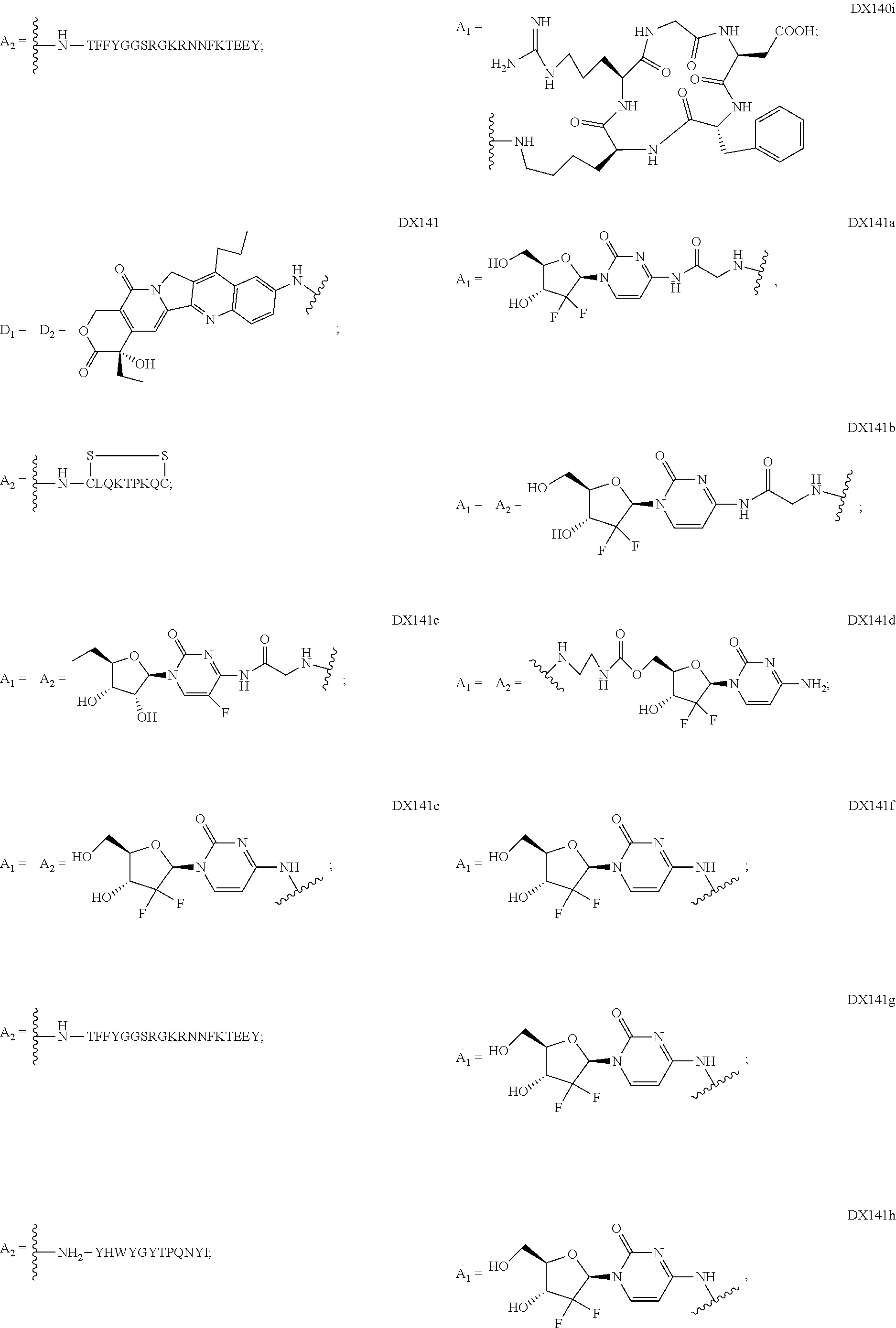 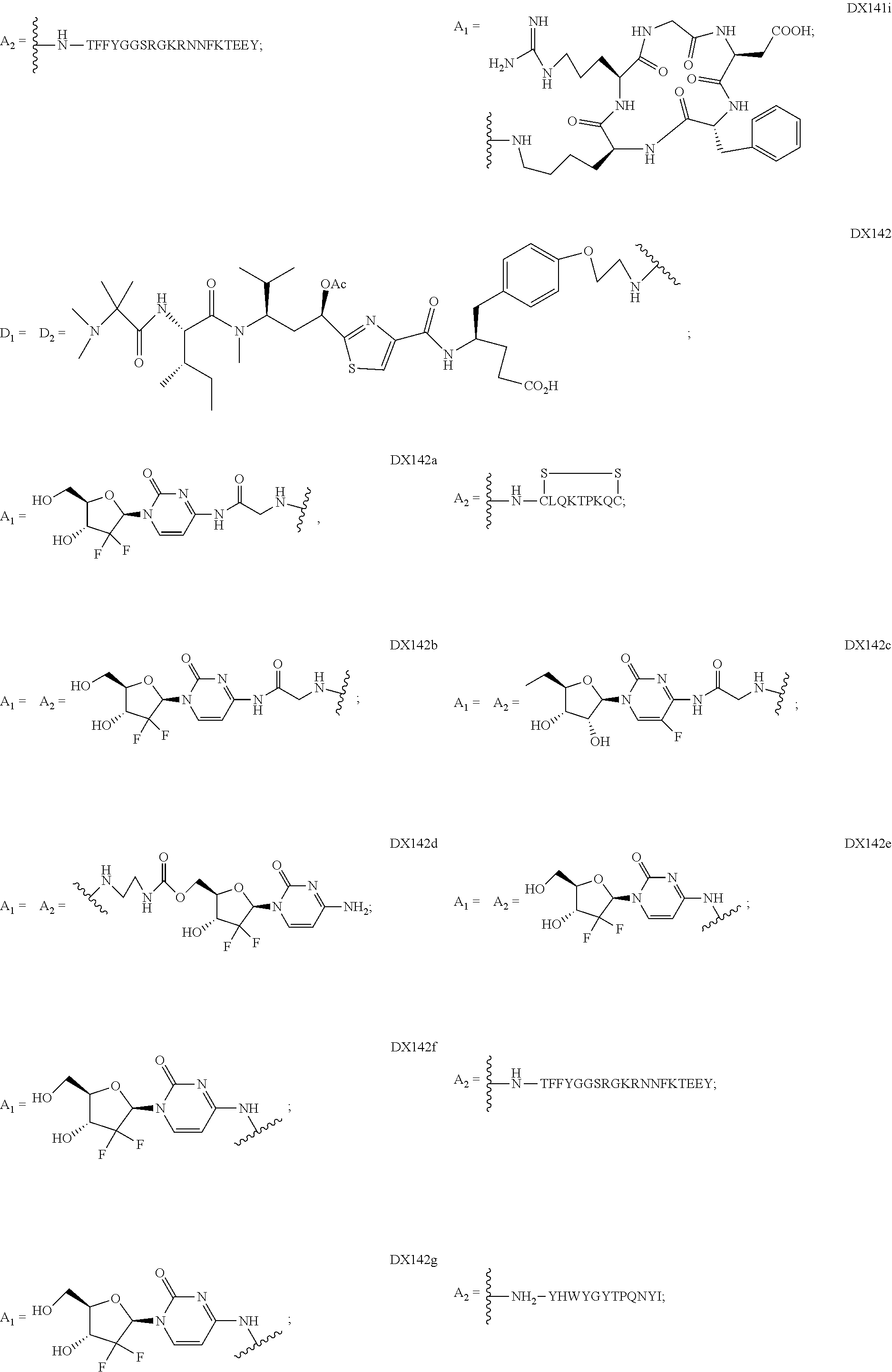 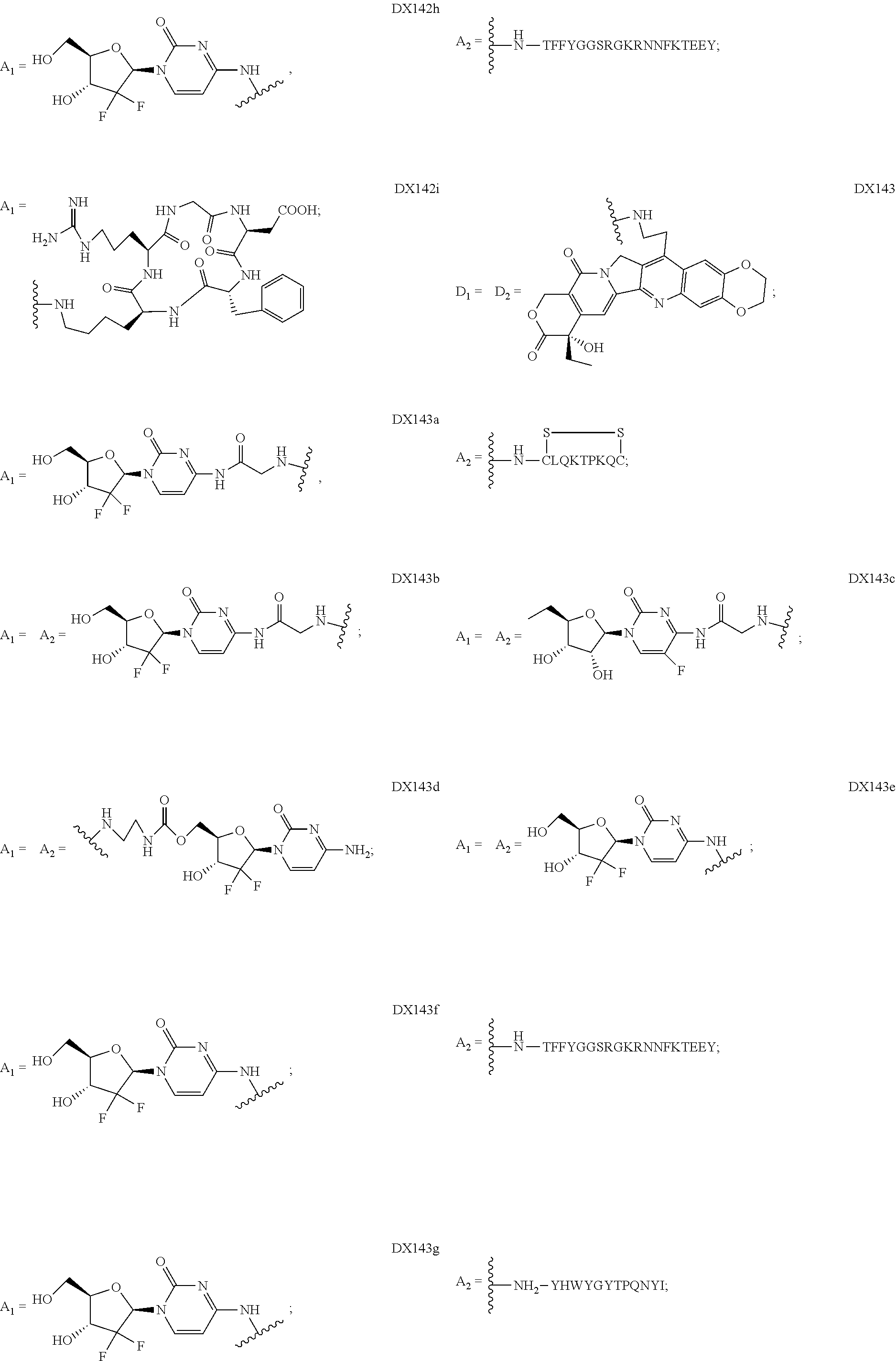 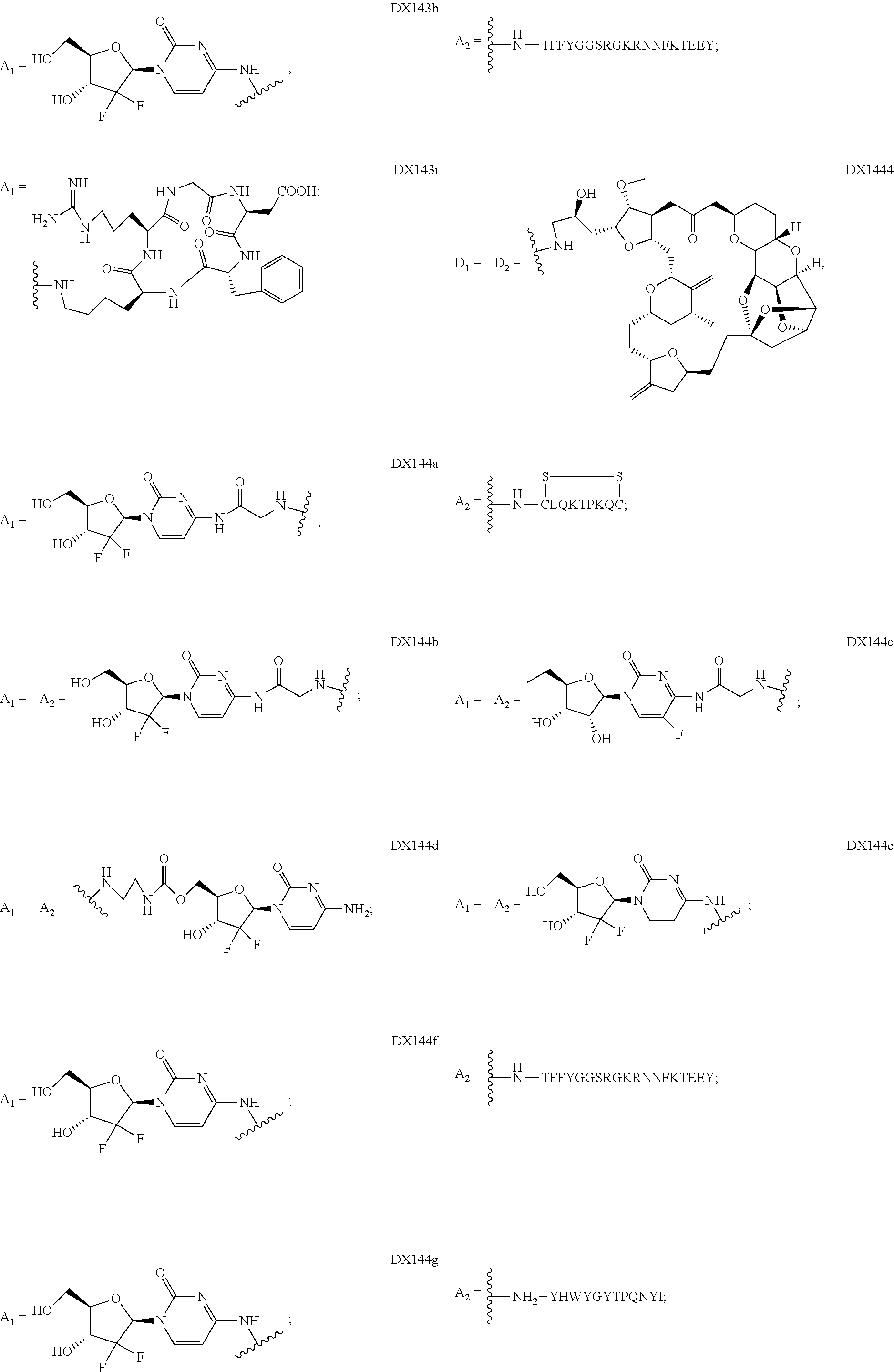 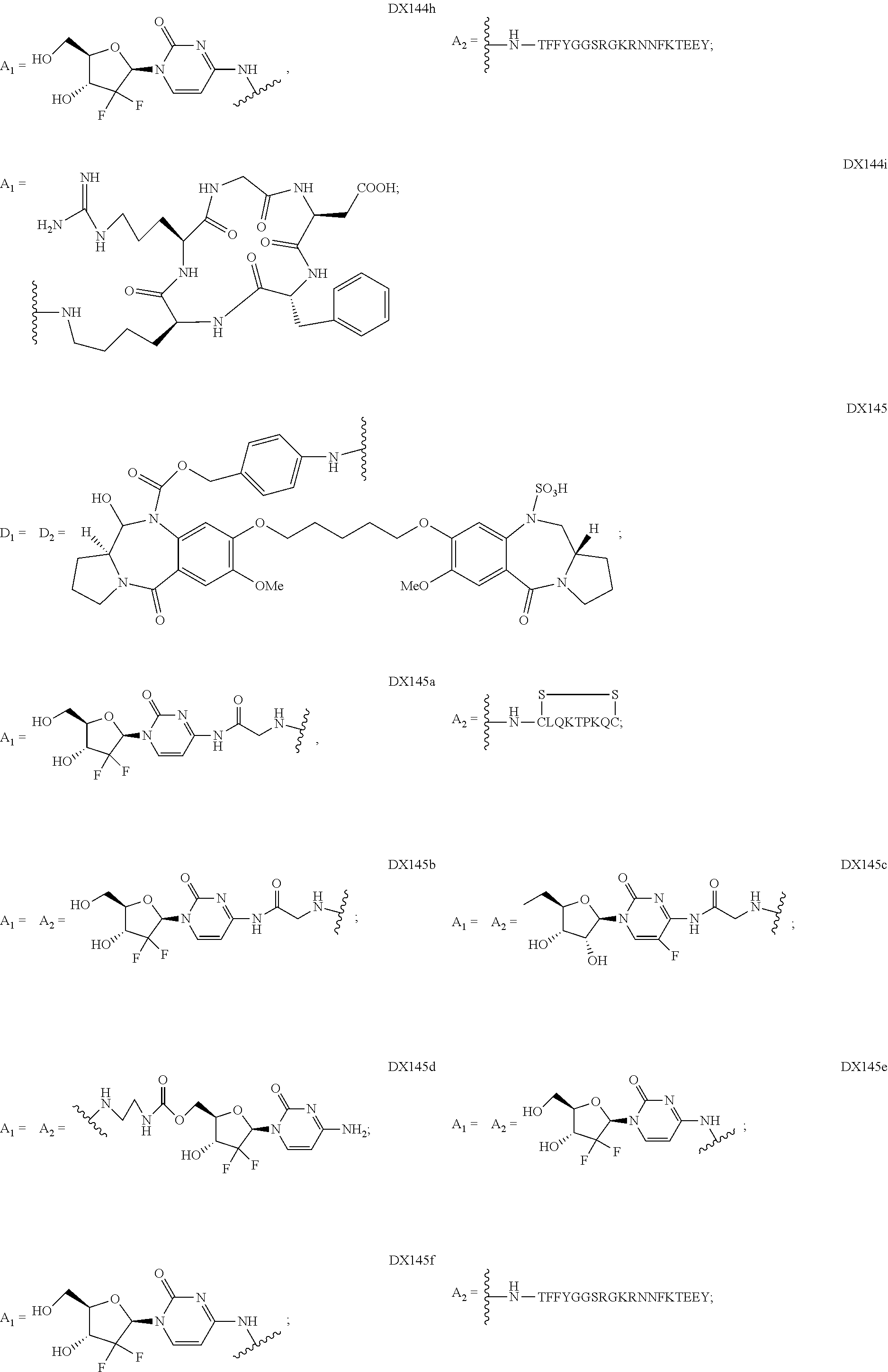 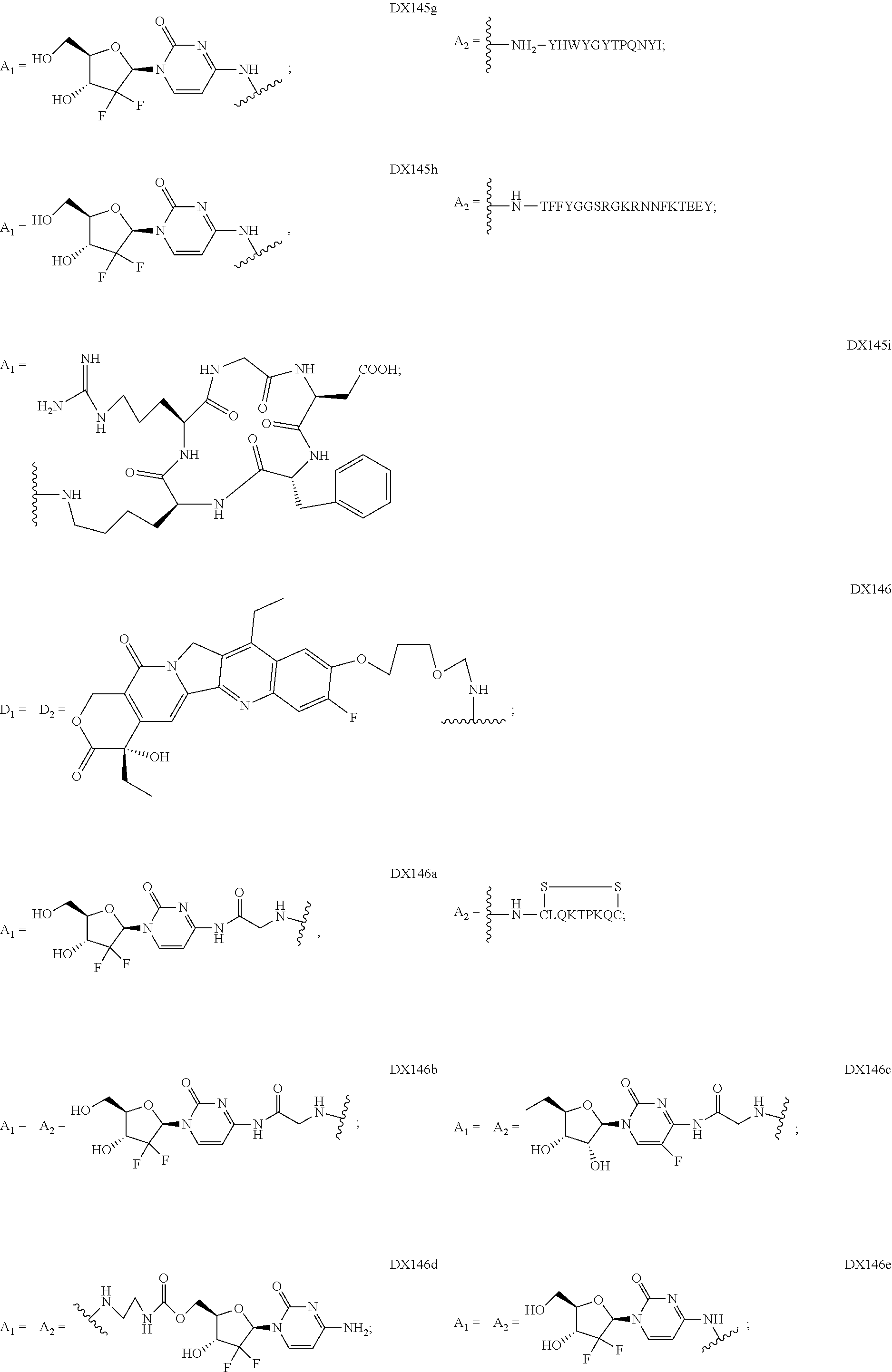 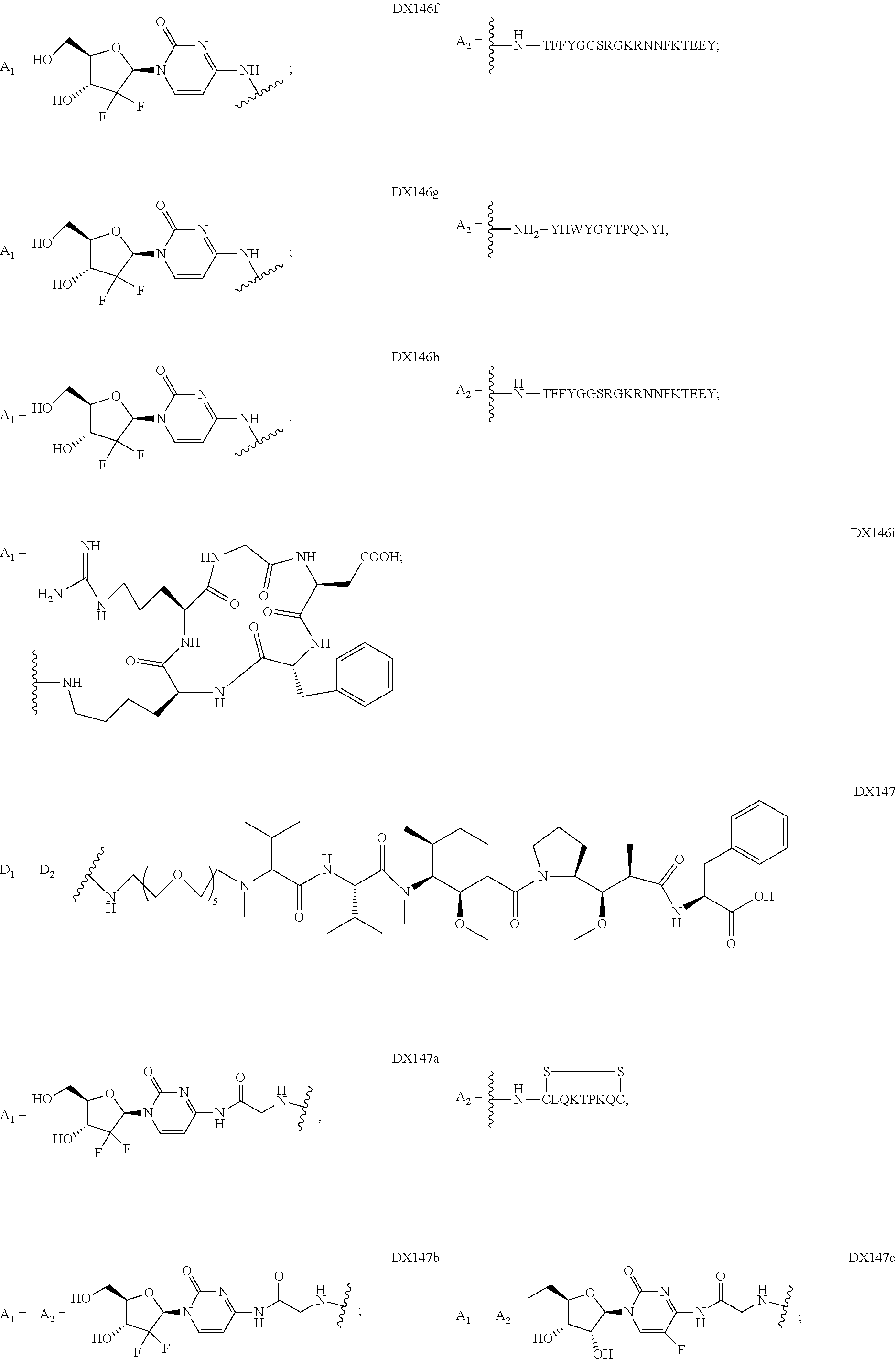 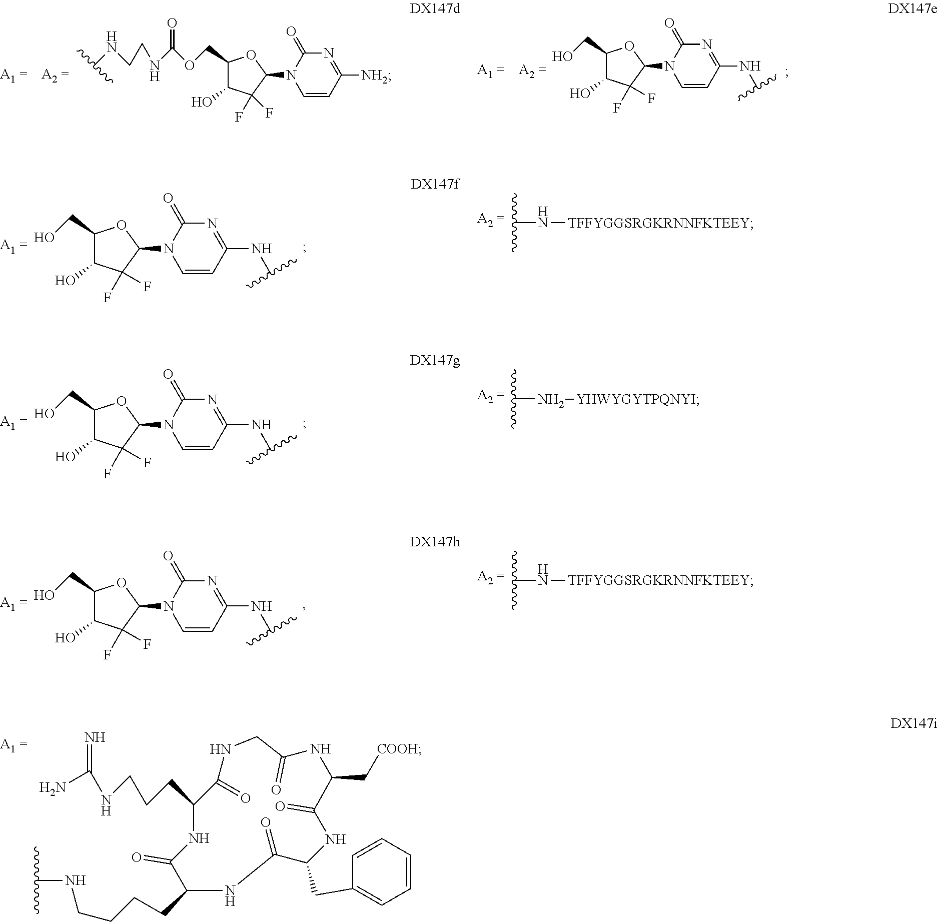 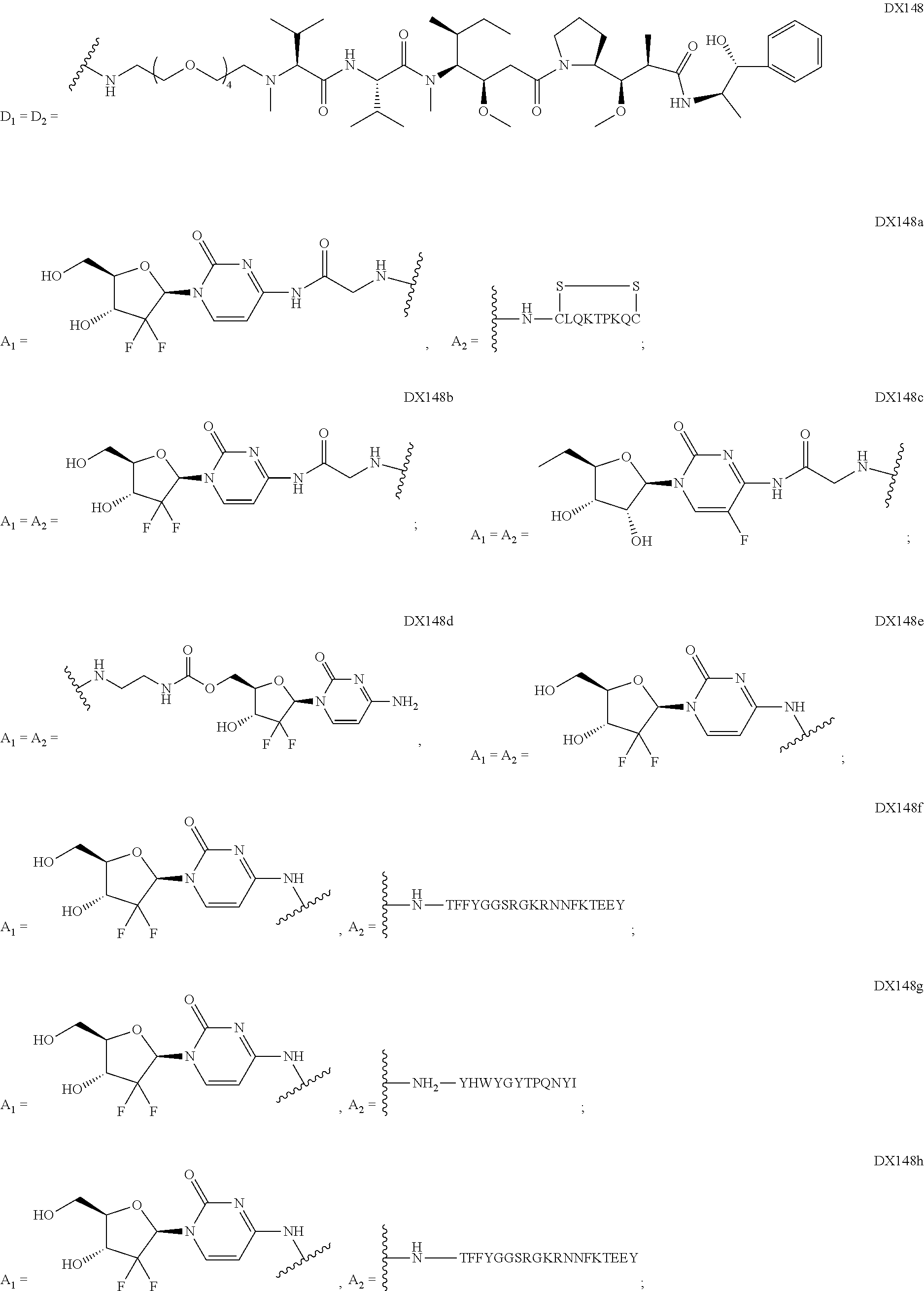 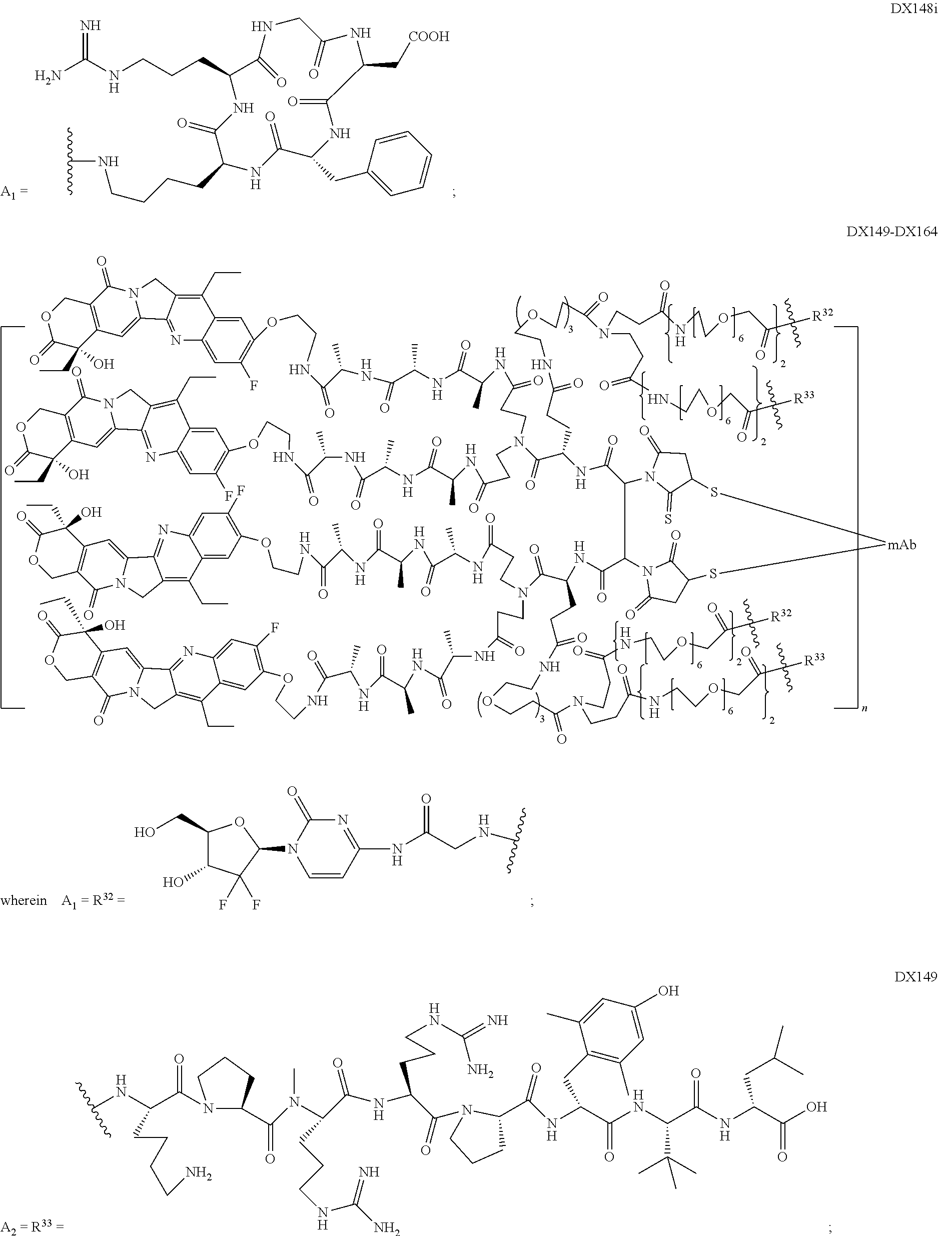 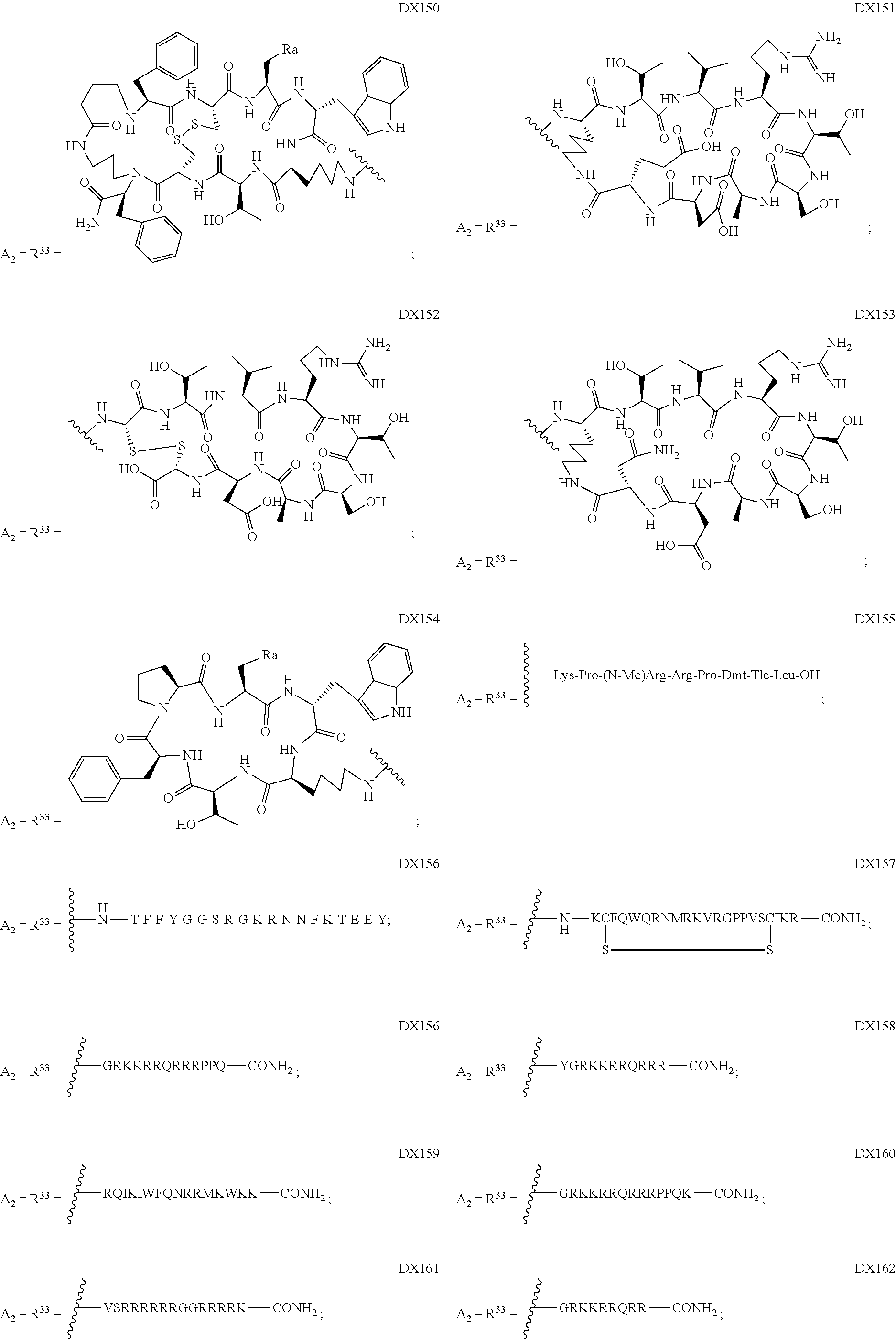 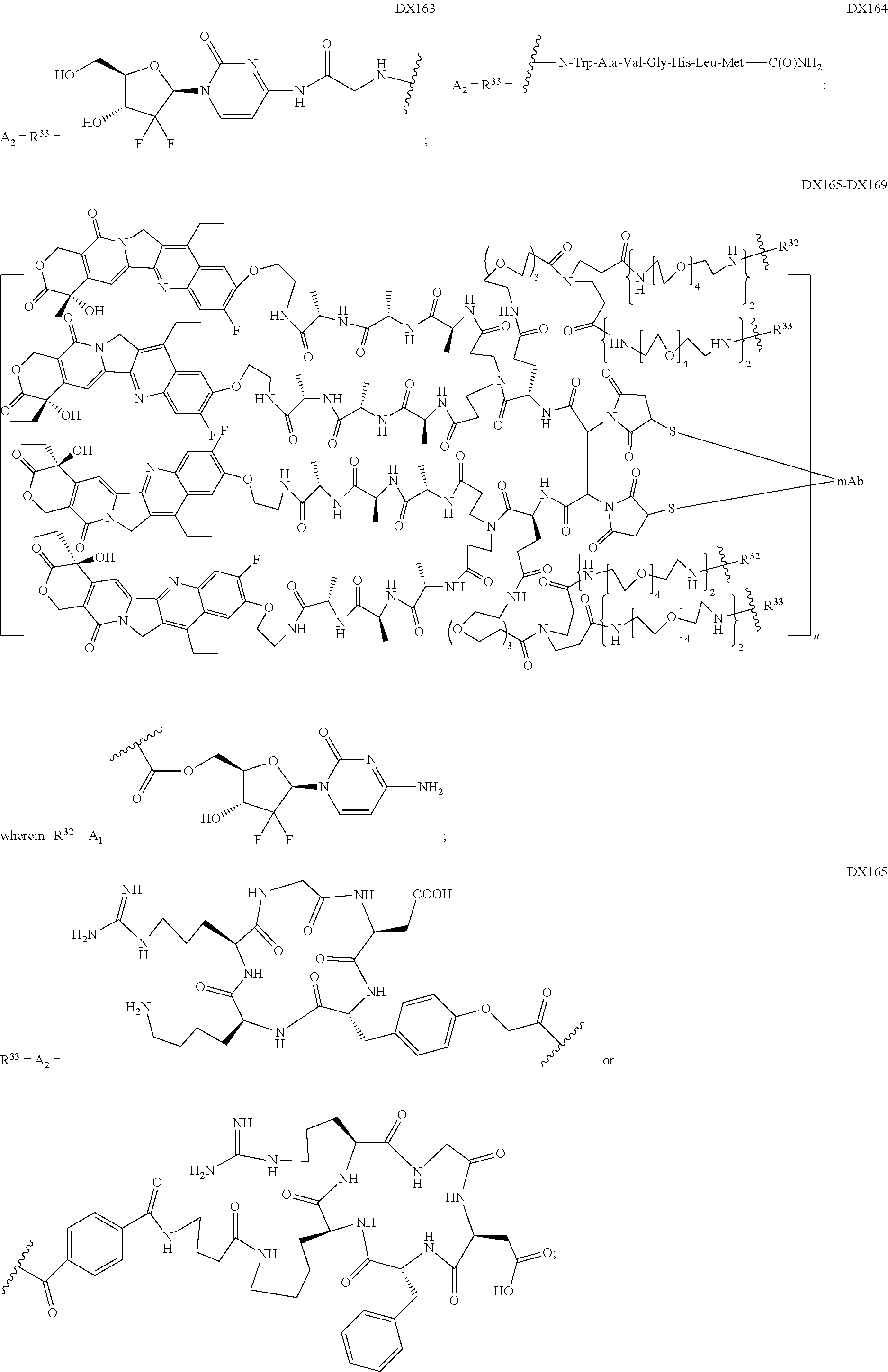 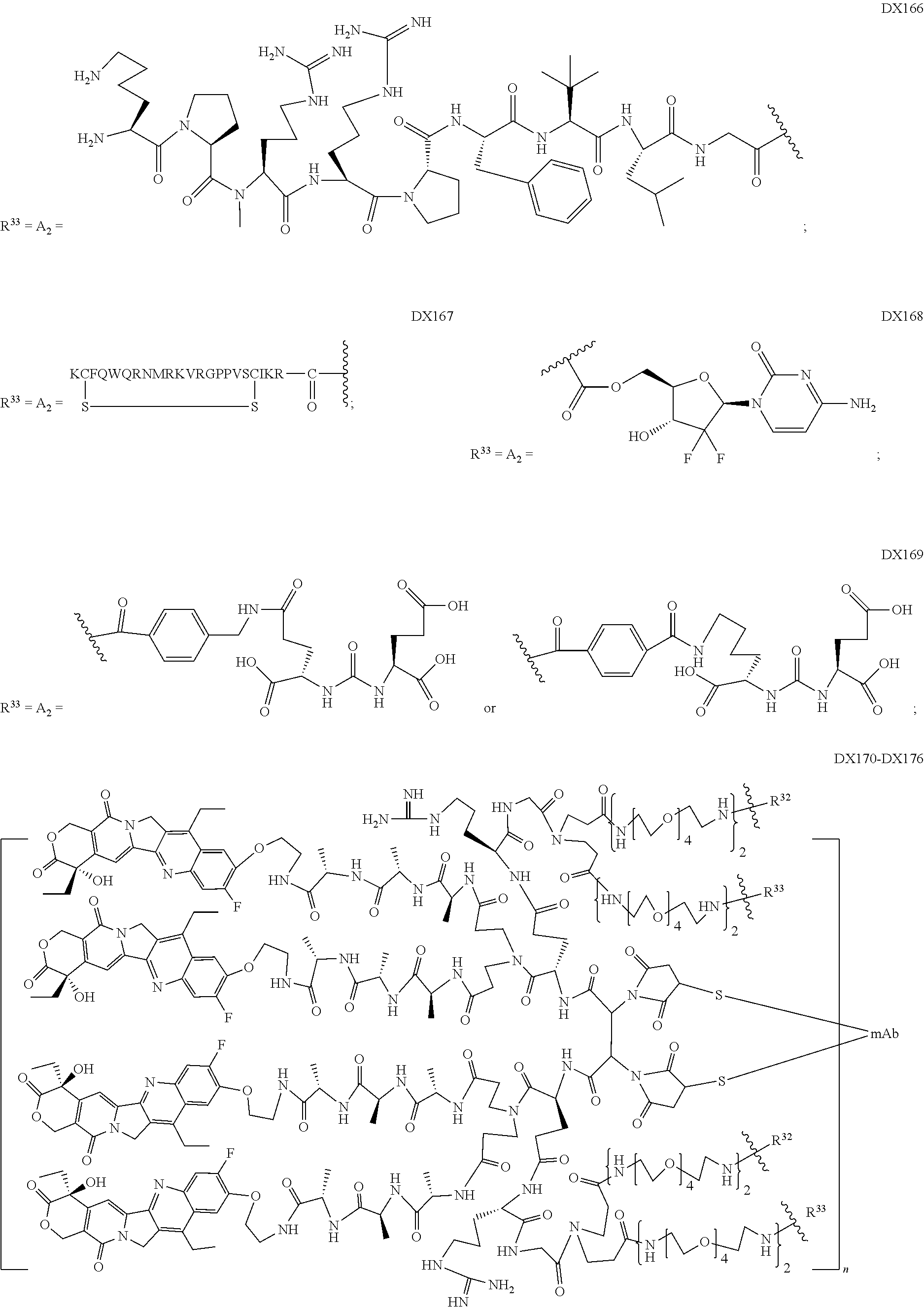 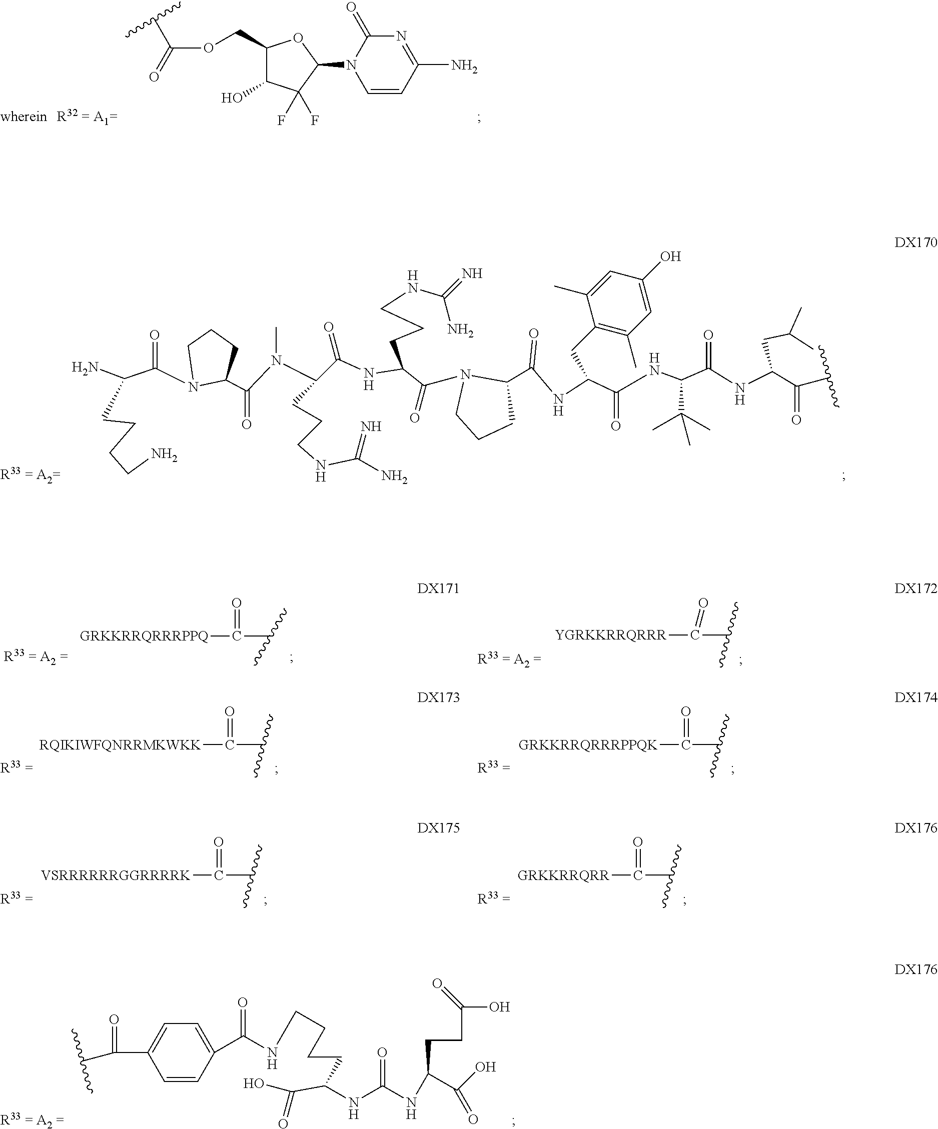 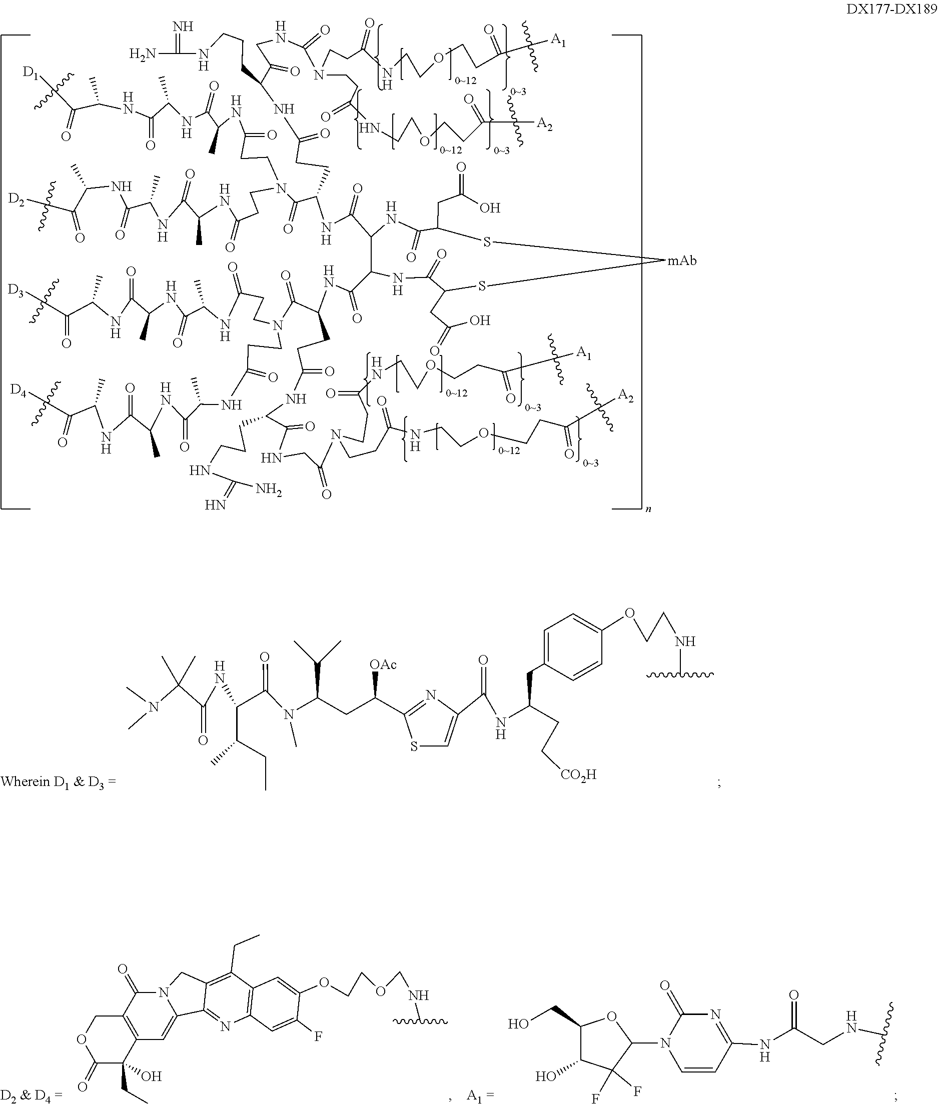 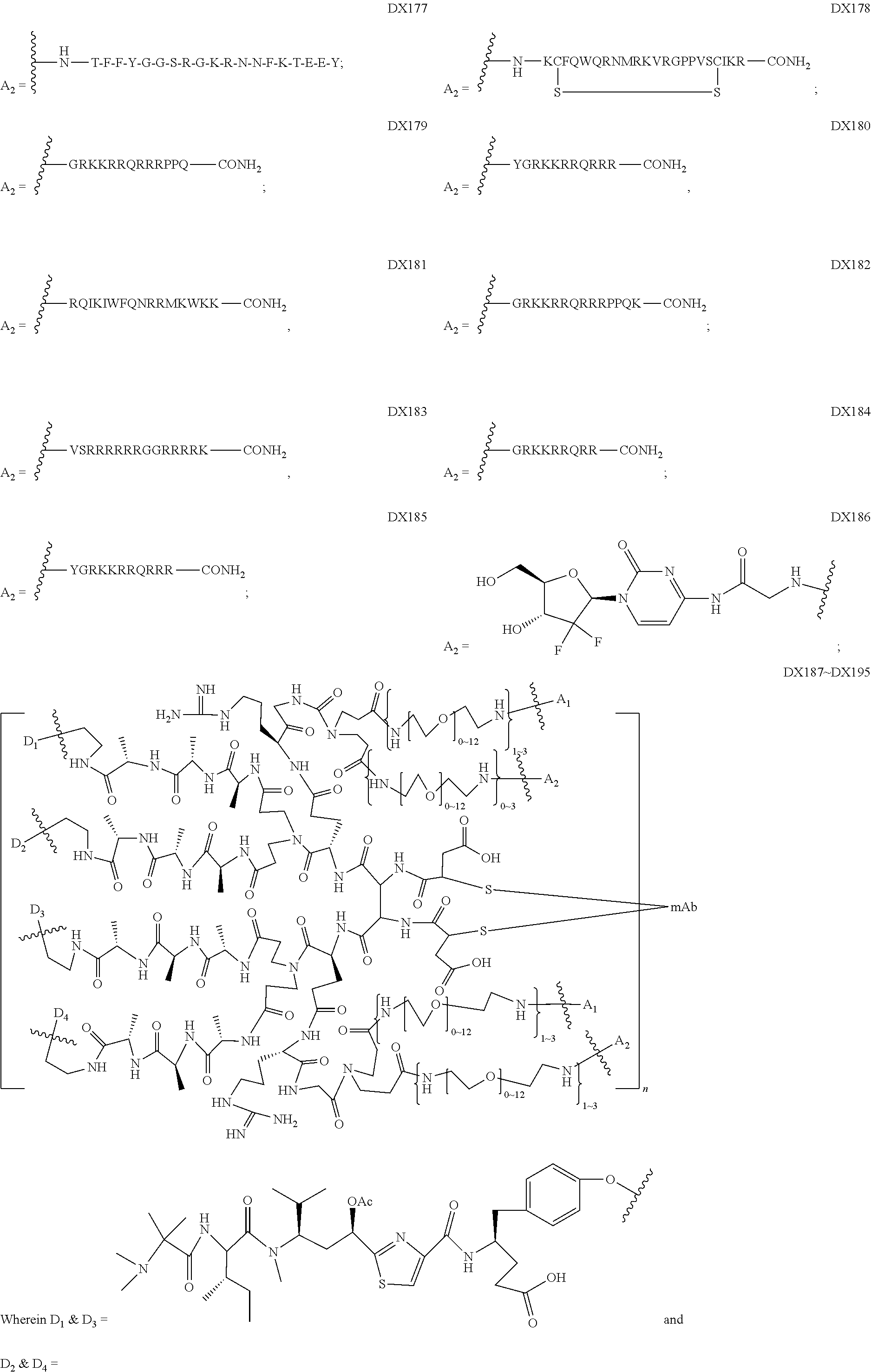 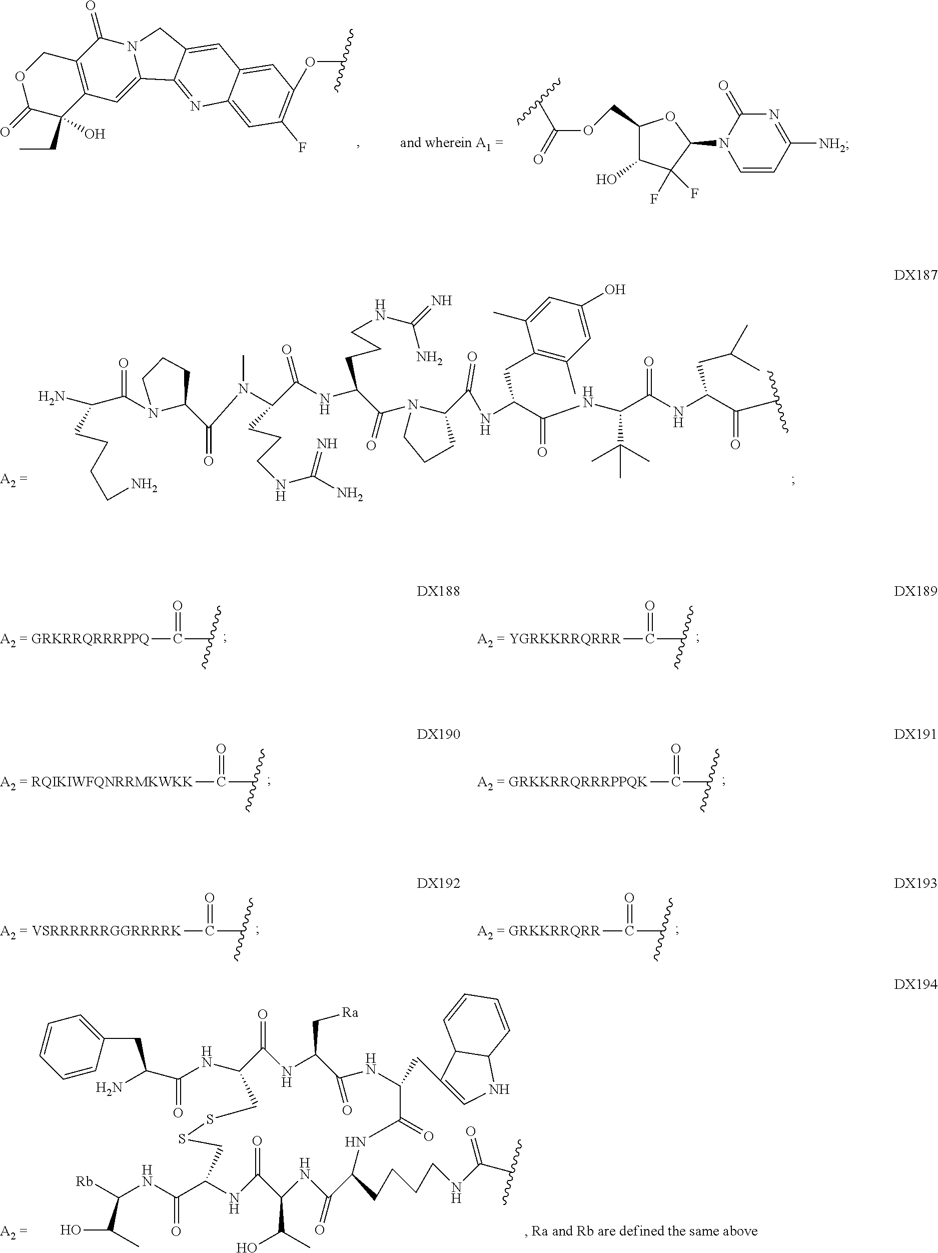 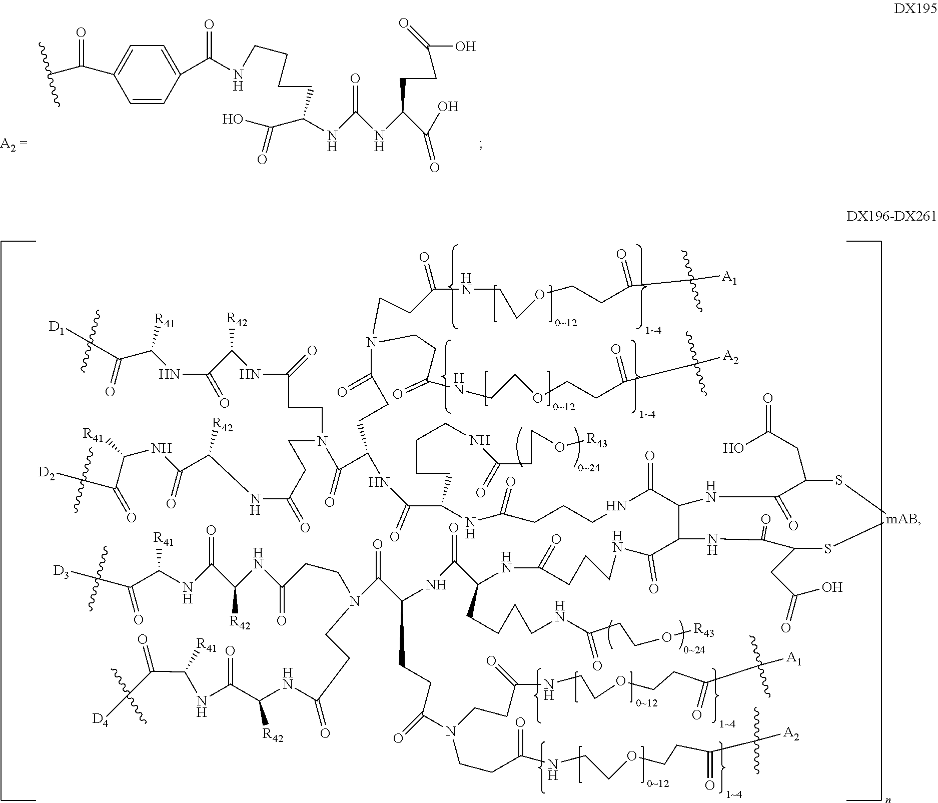
wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H4OH; R₄₃ is H, CH₃, CH₂CH₃, C(O)H, and C(O)CH₃; D1, D₂, D3 and D4 are cytotoxic drug and A₁ and A₂ are small molecular ligands or drugs, 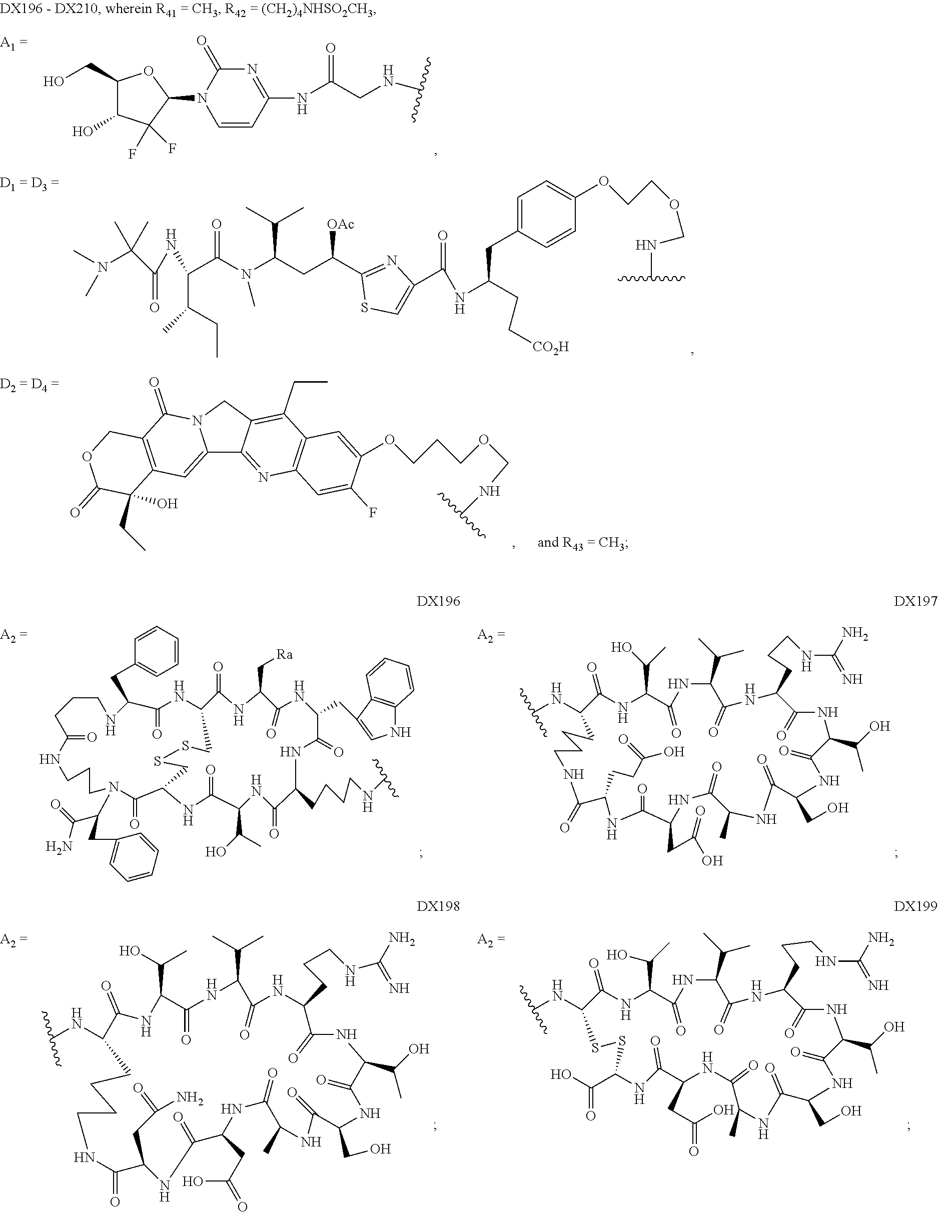 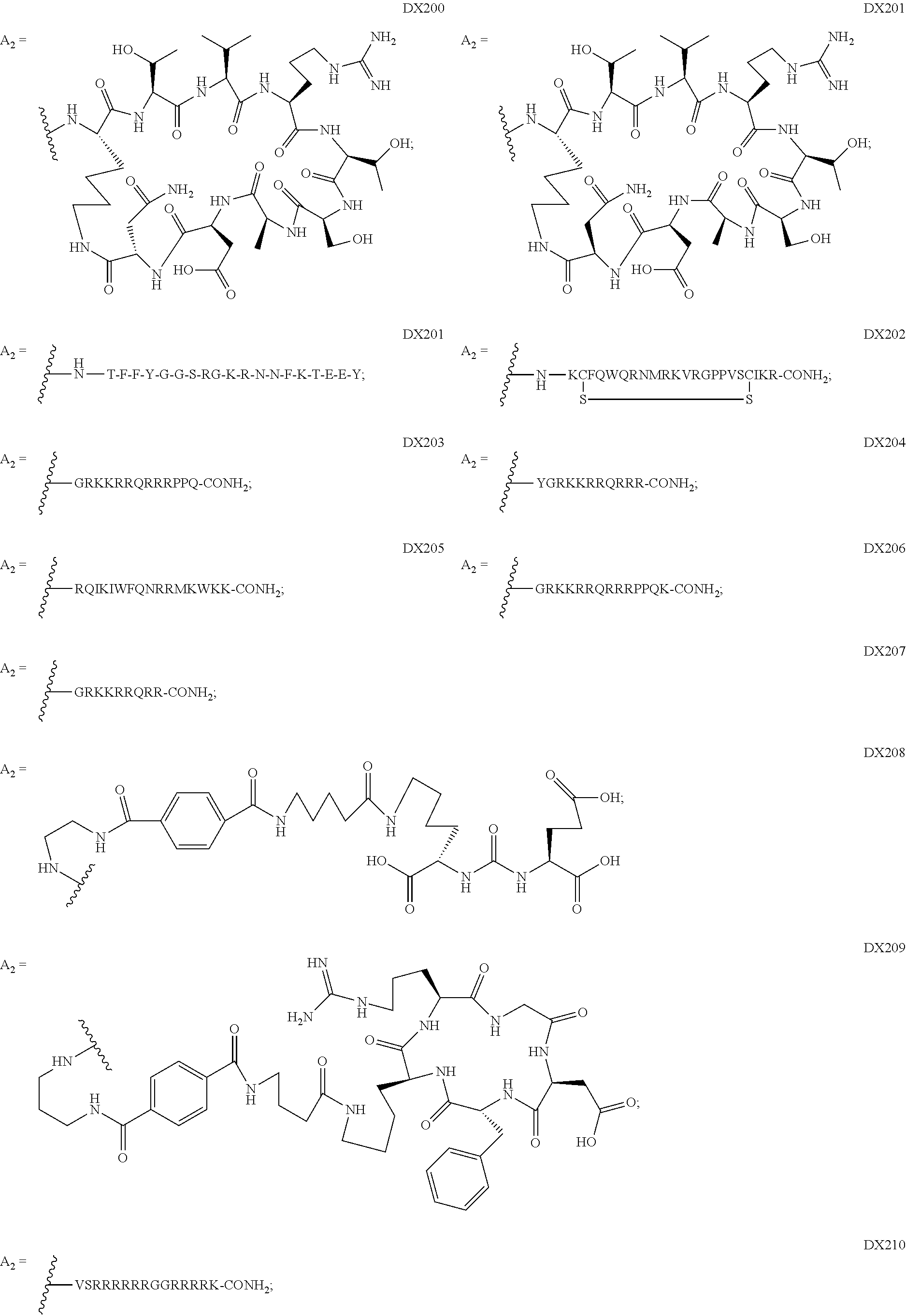 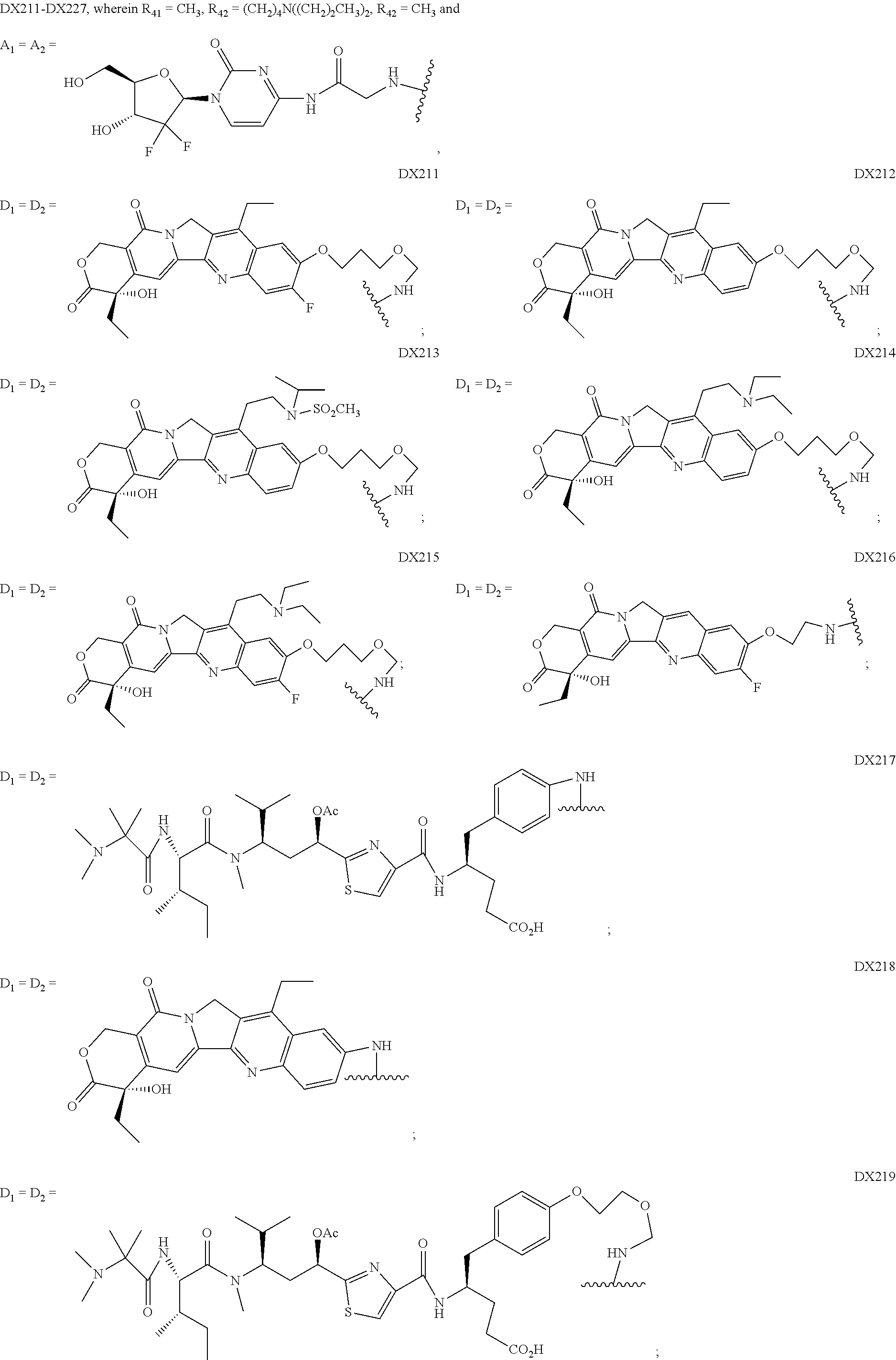 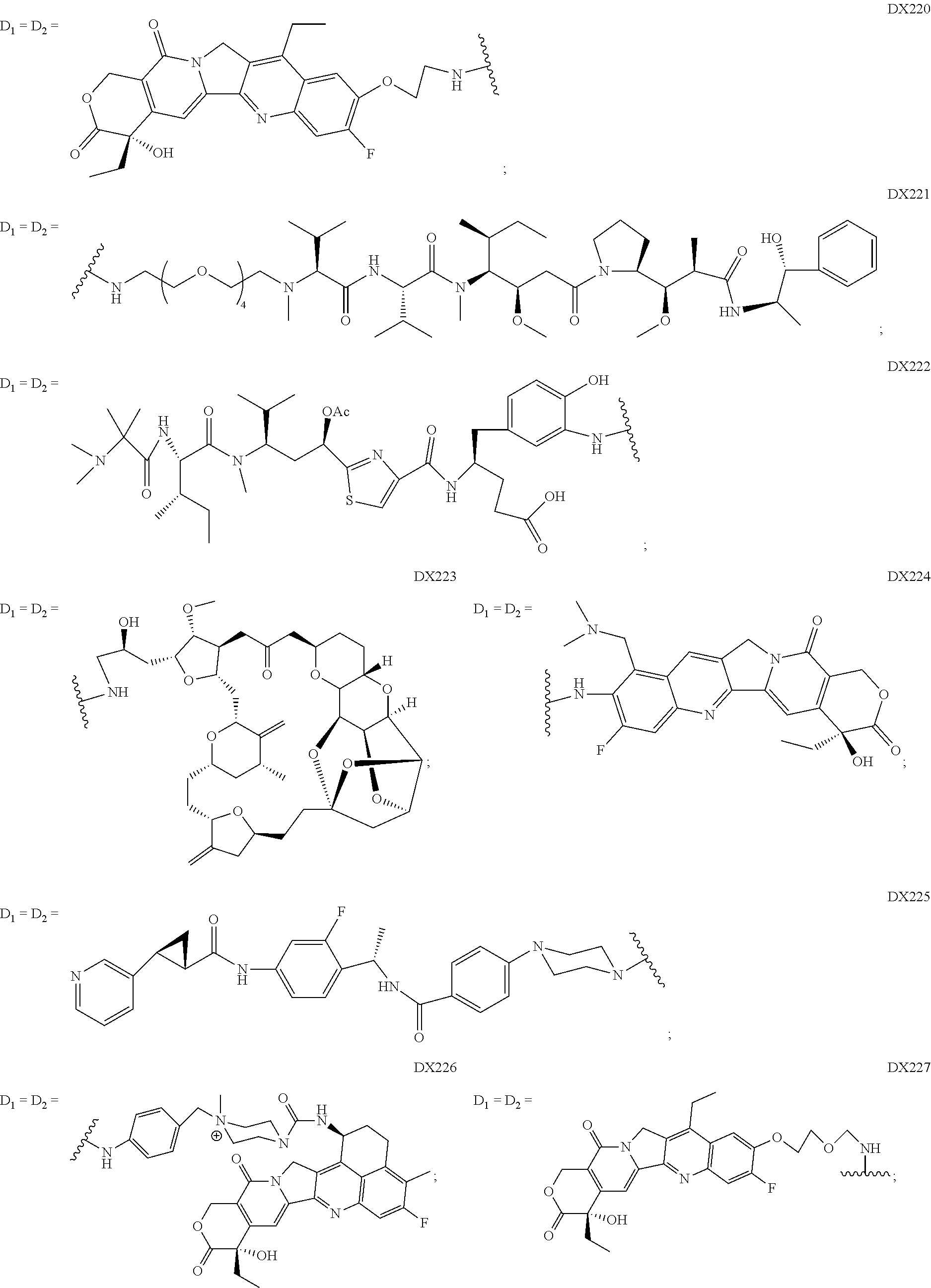 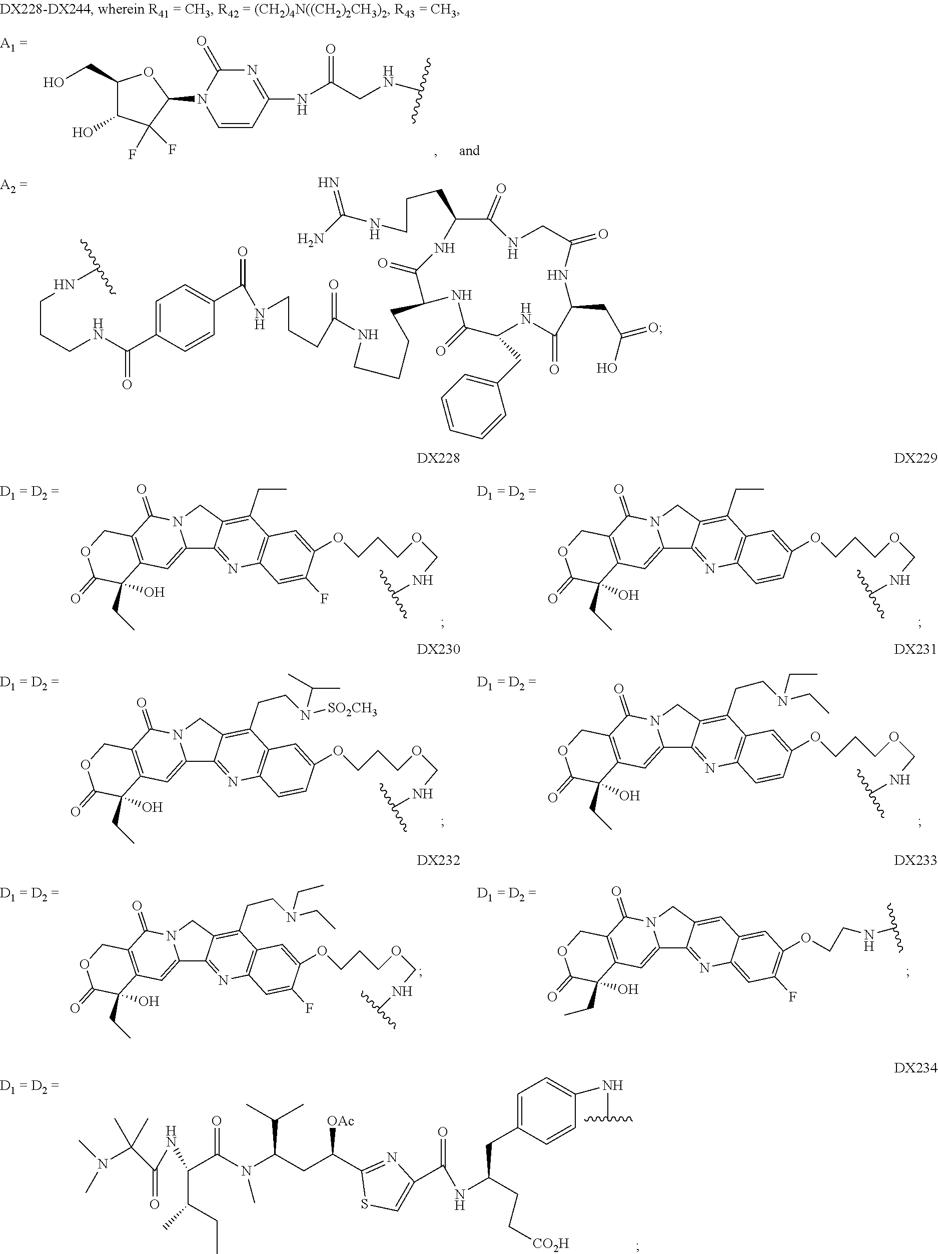 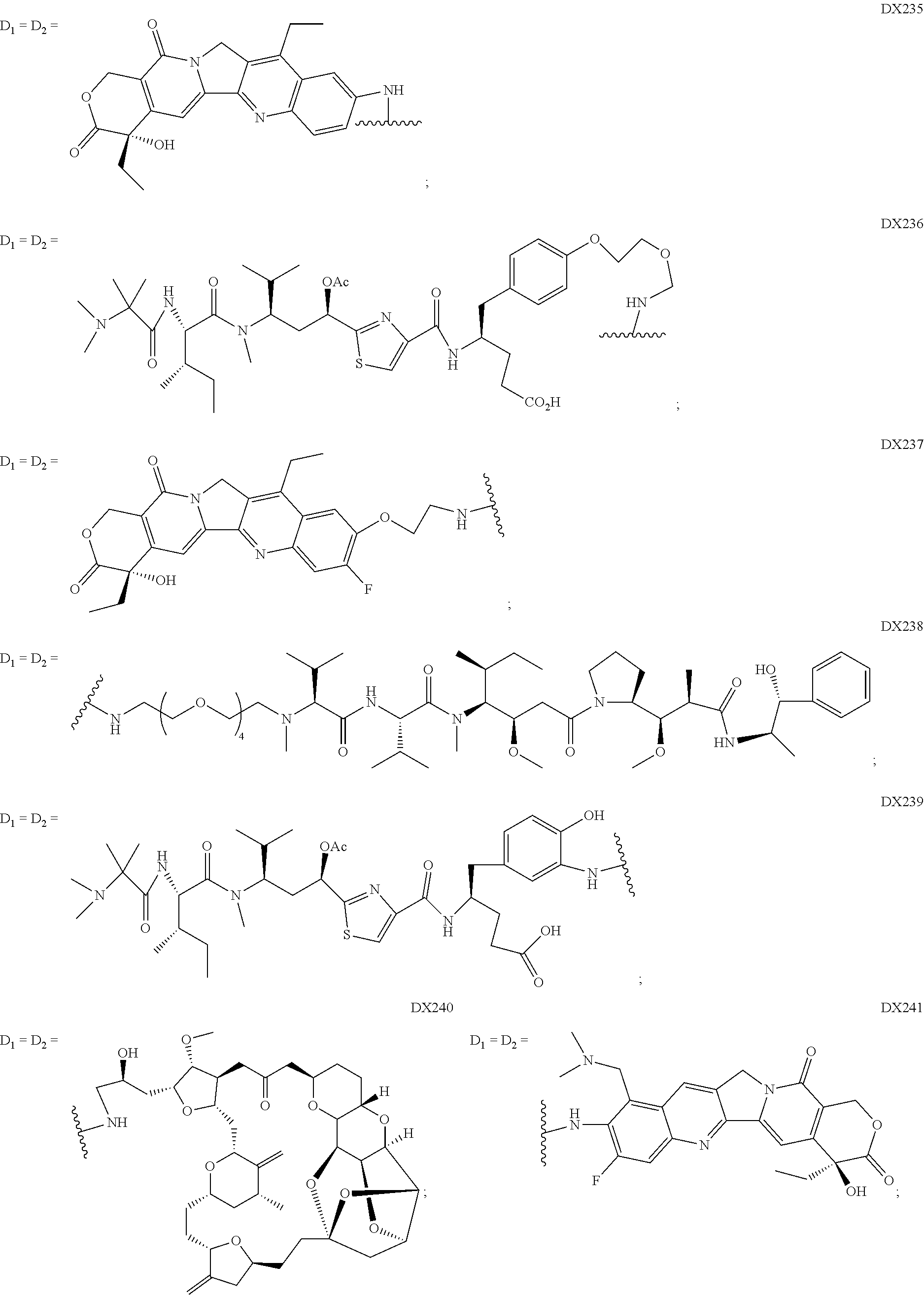 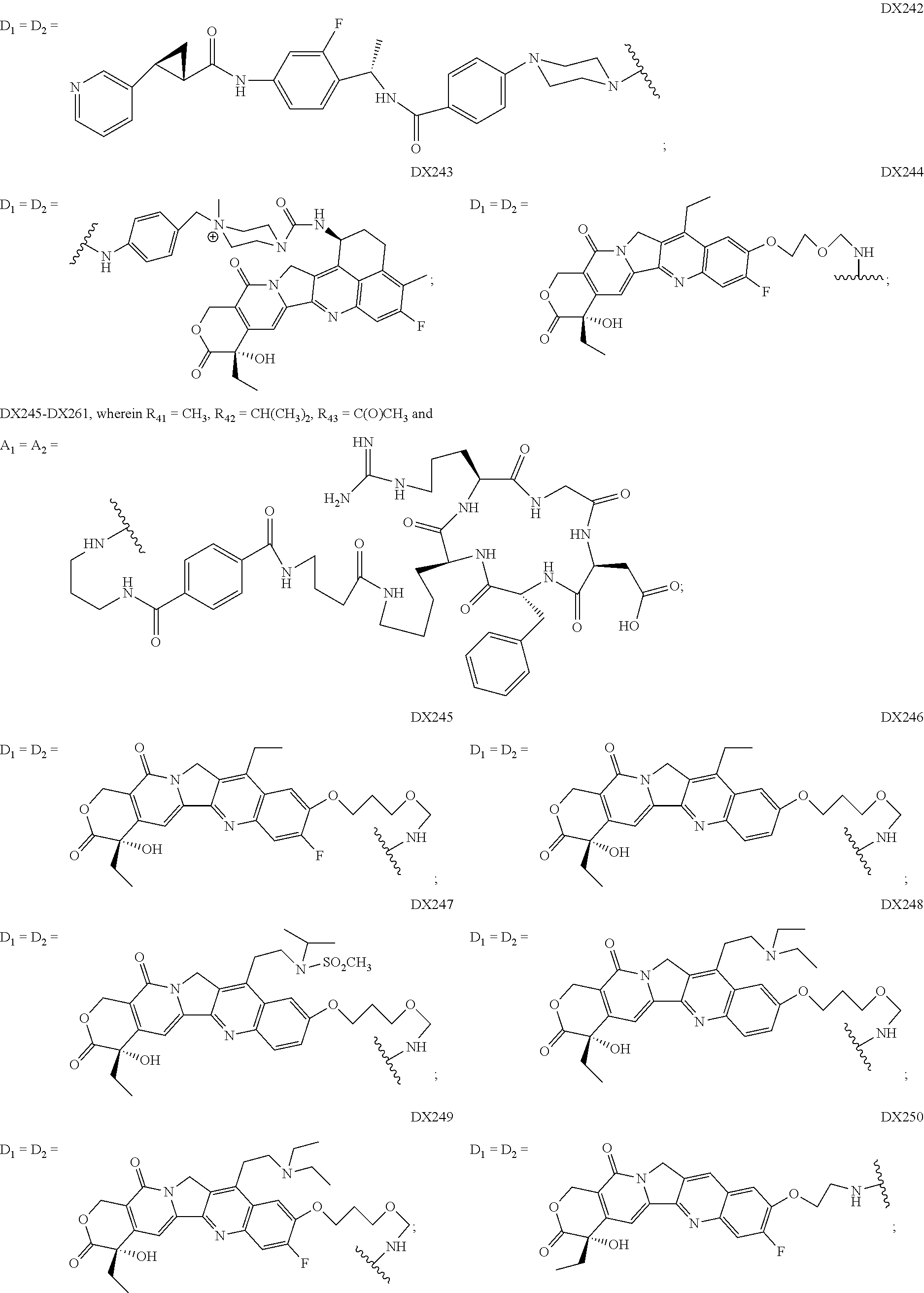 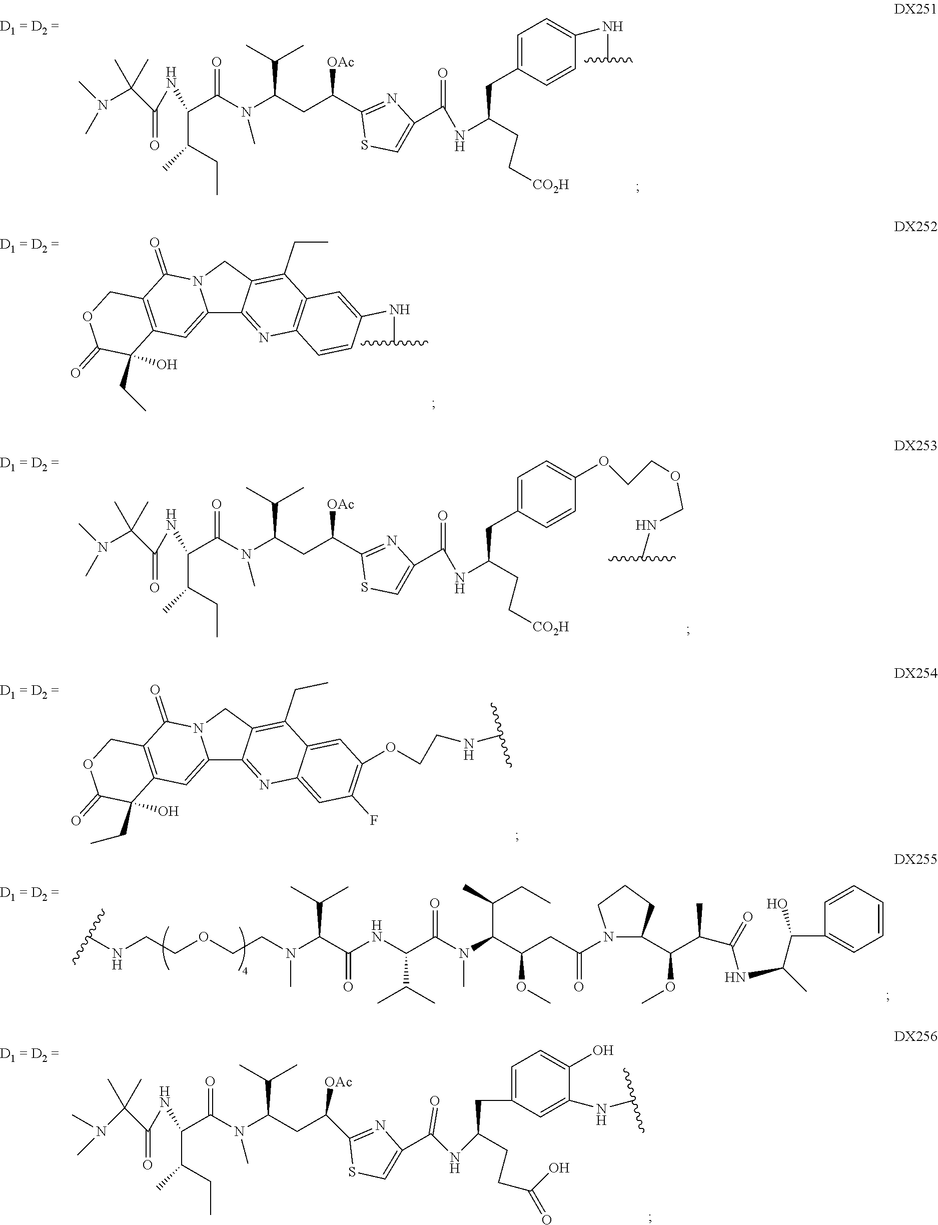 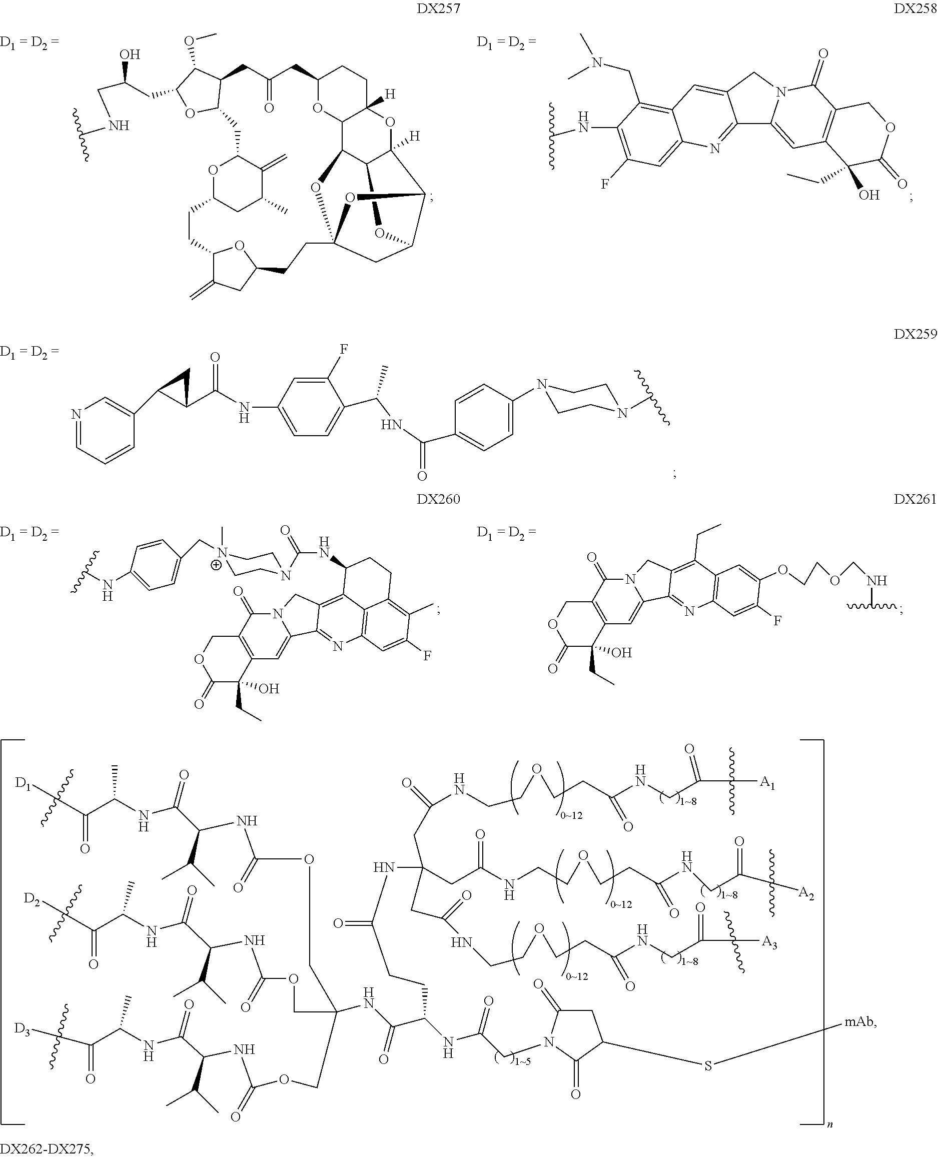 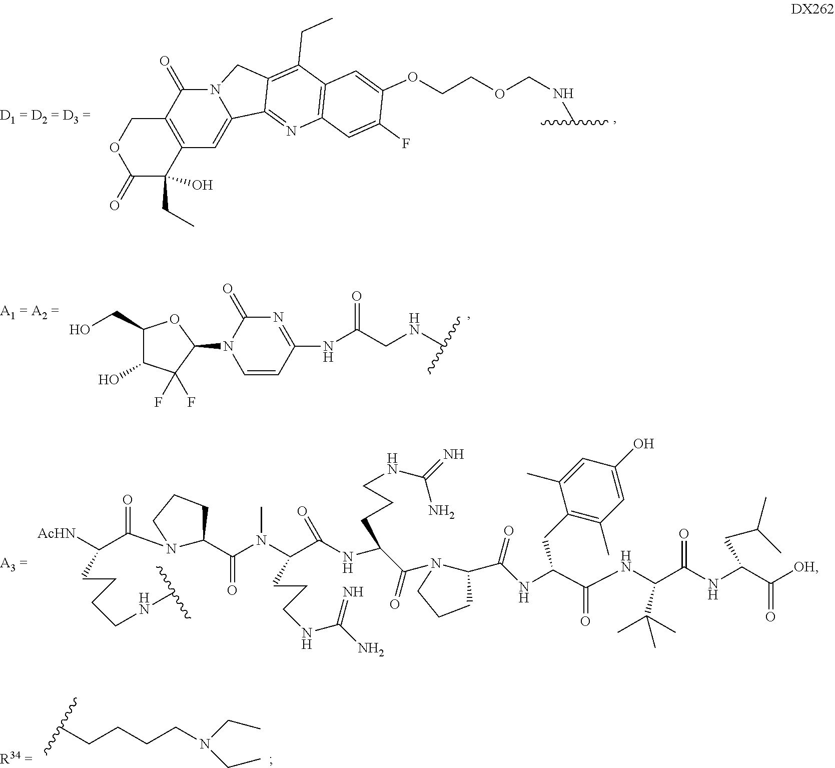 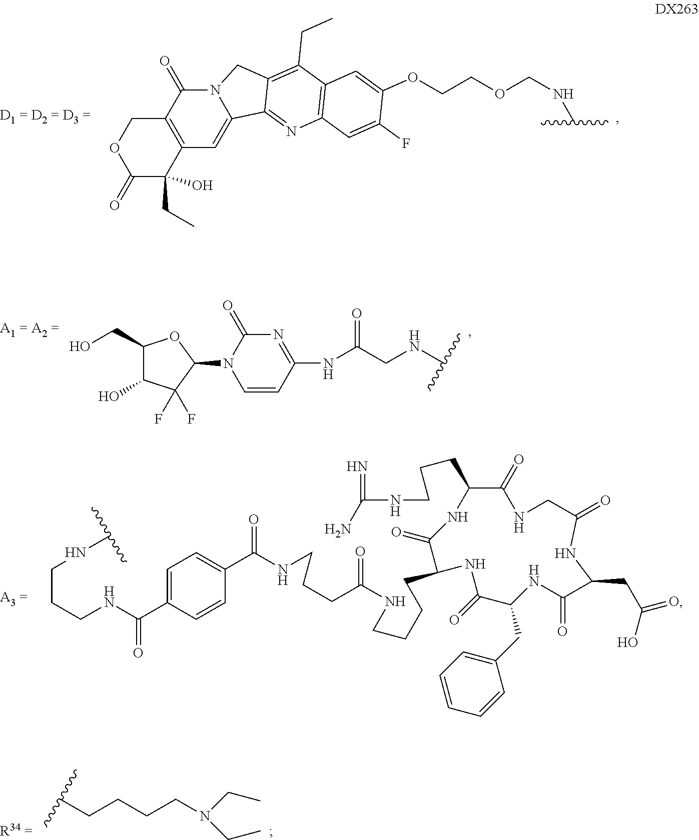 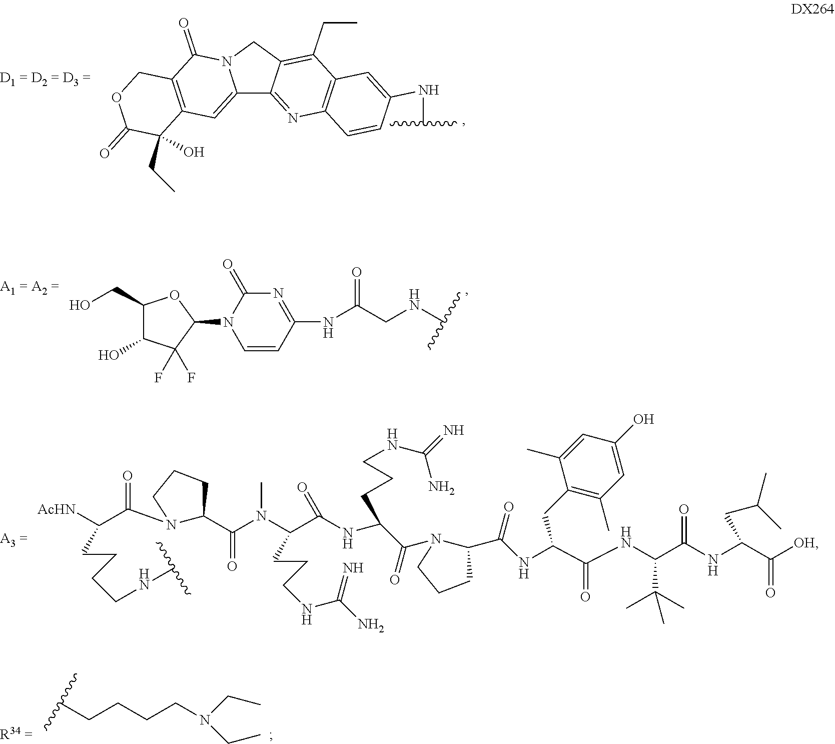 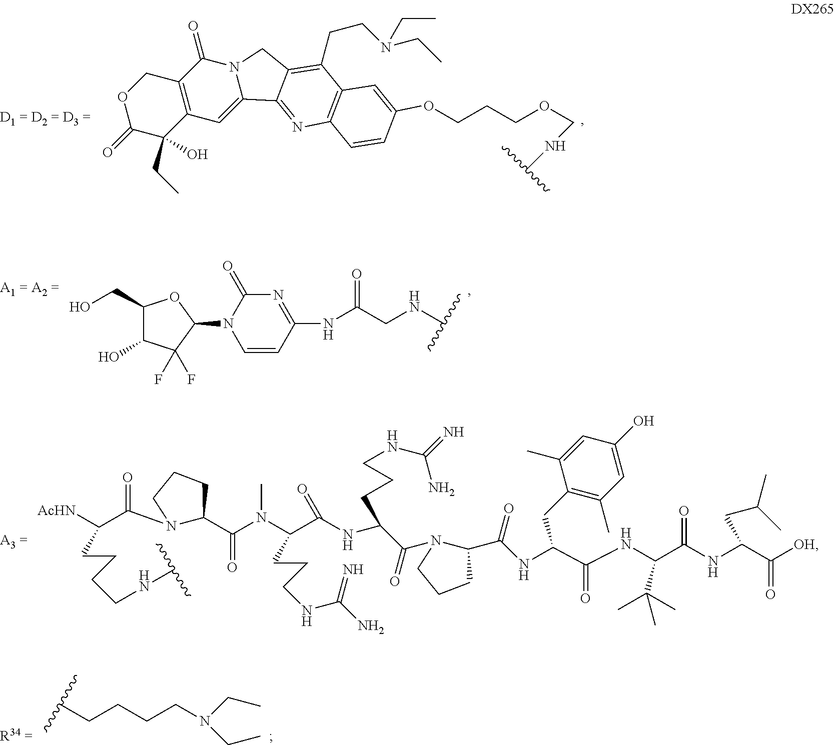 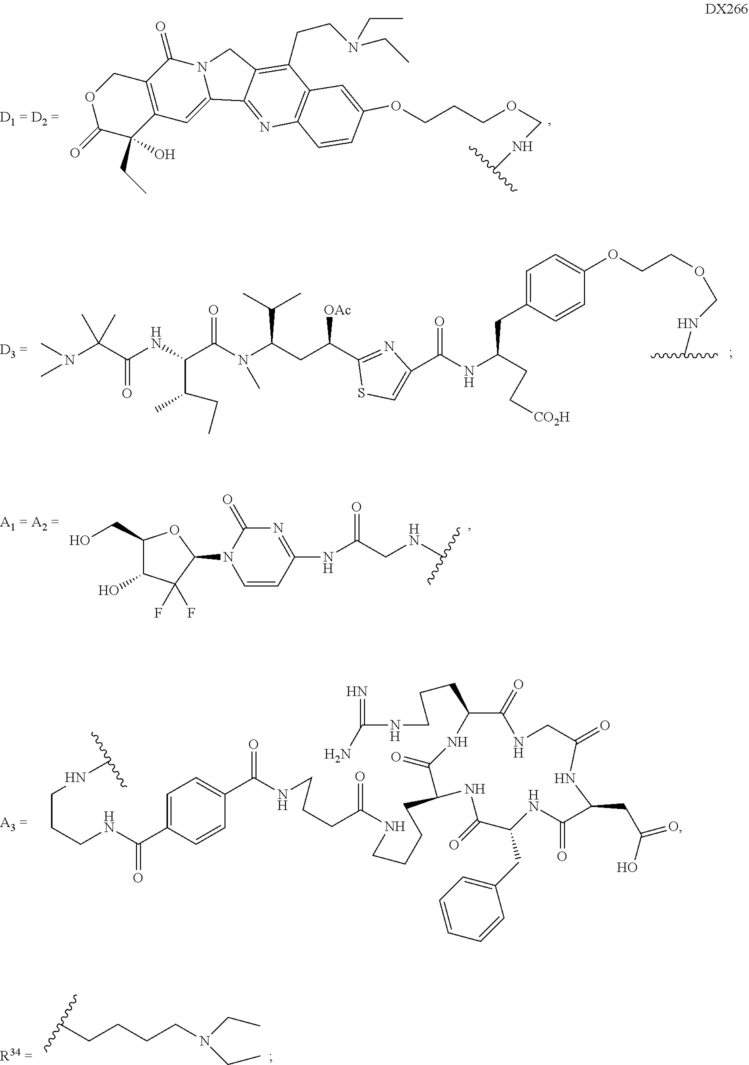 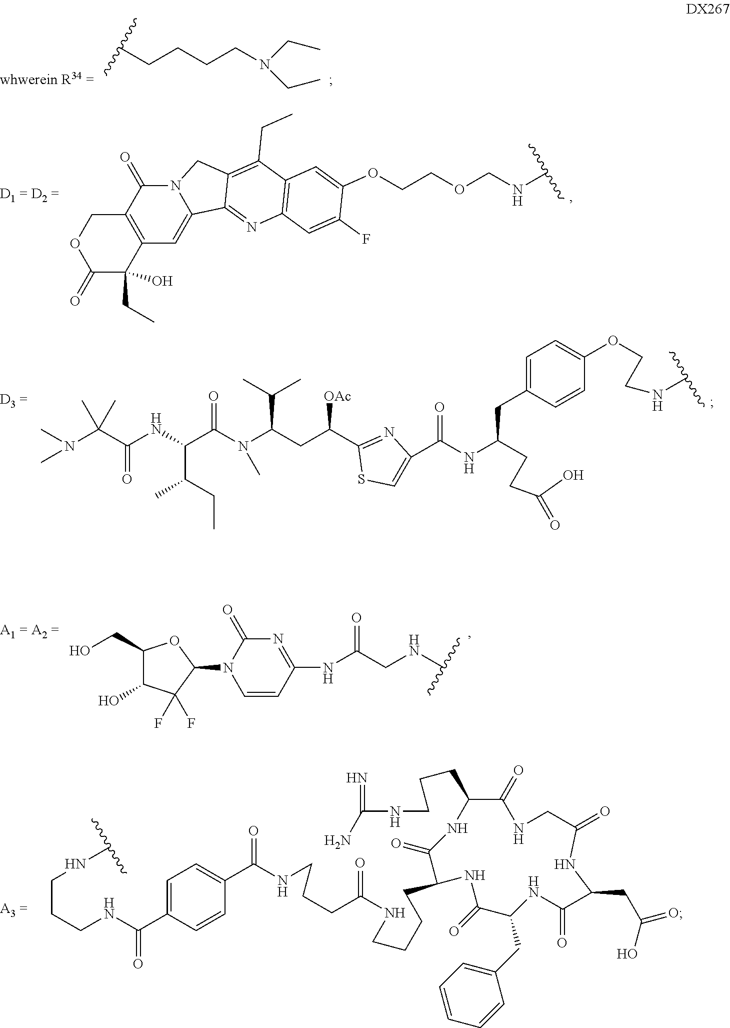 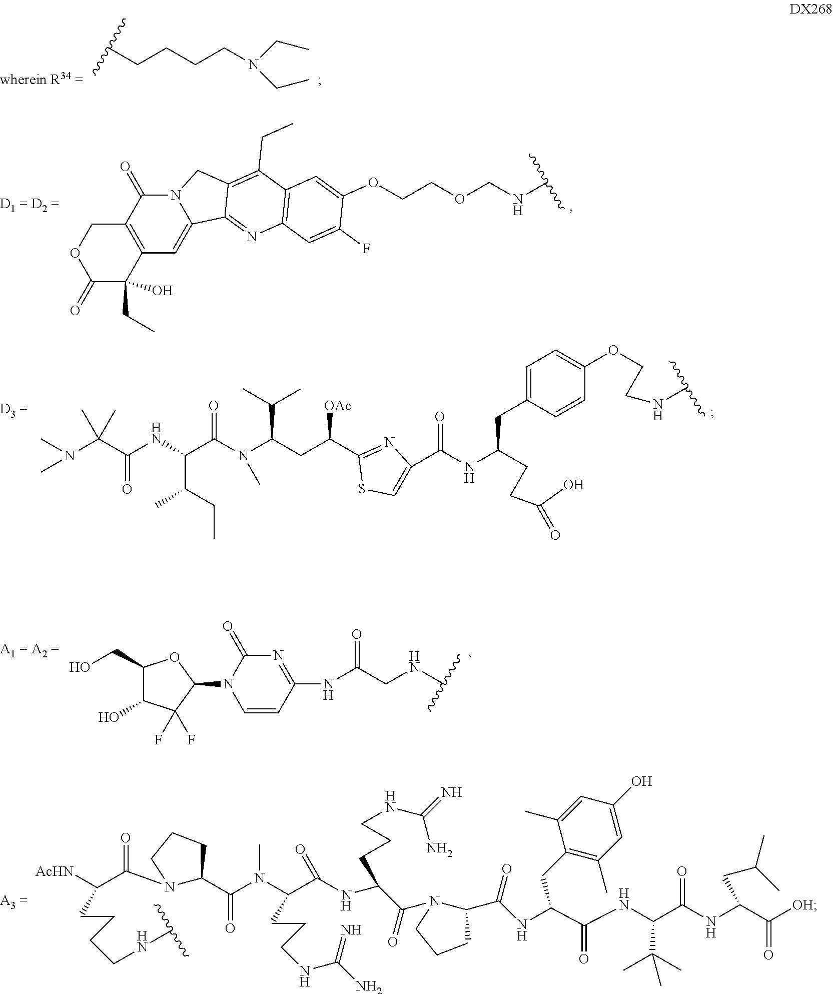 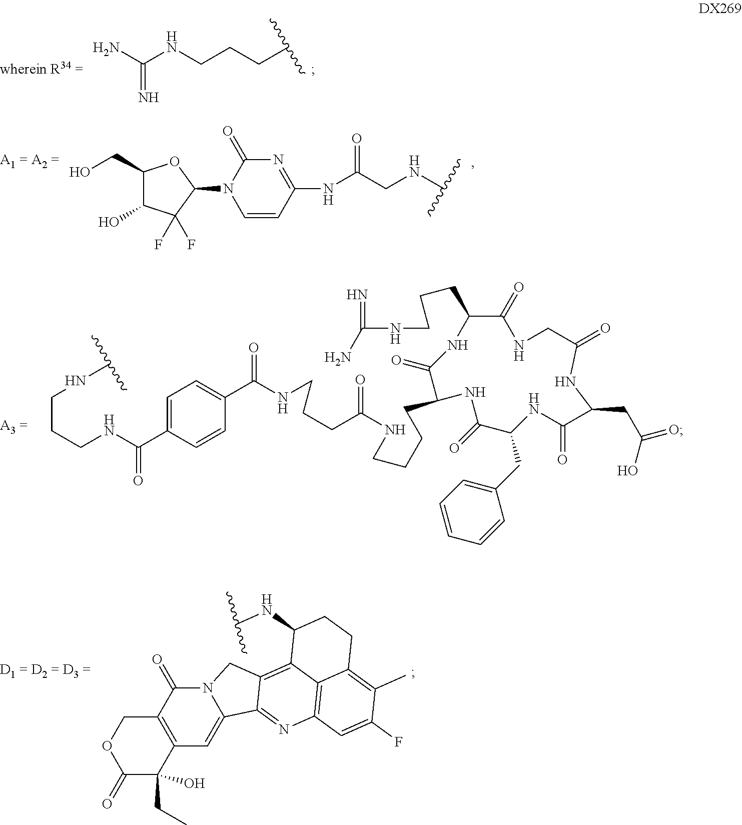 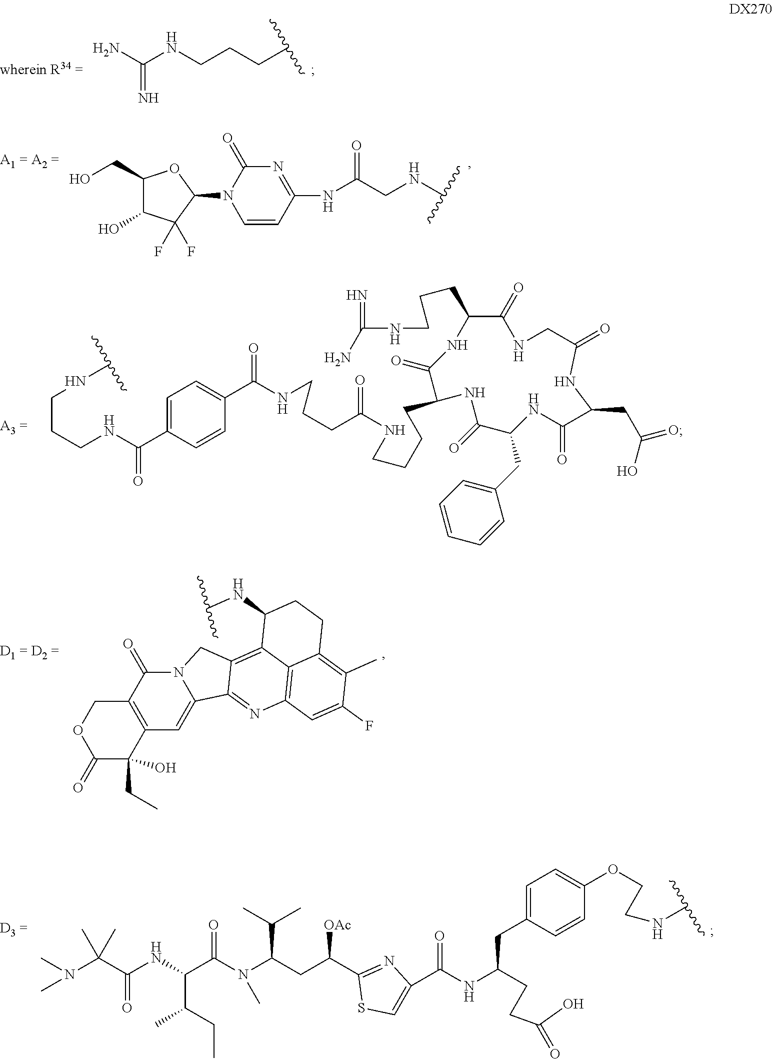 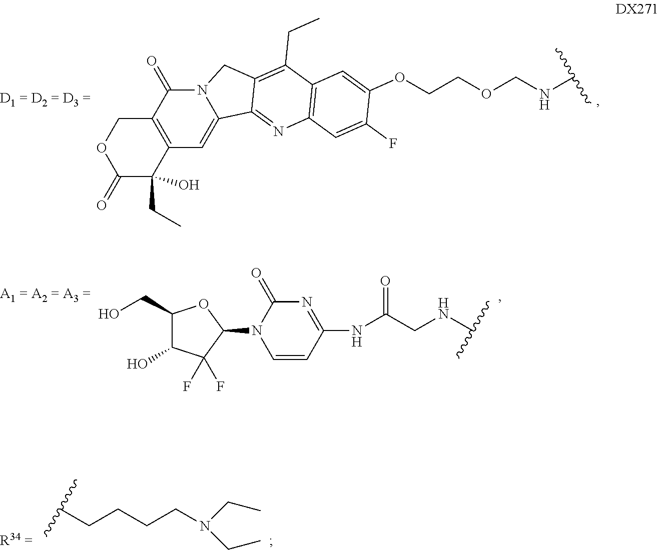 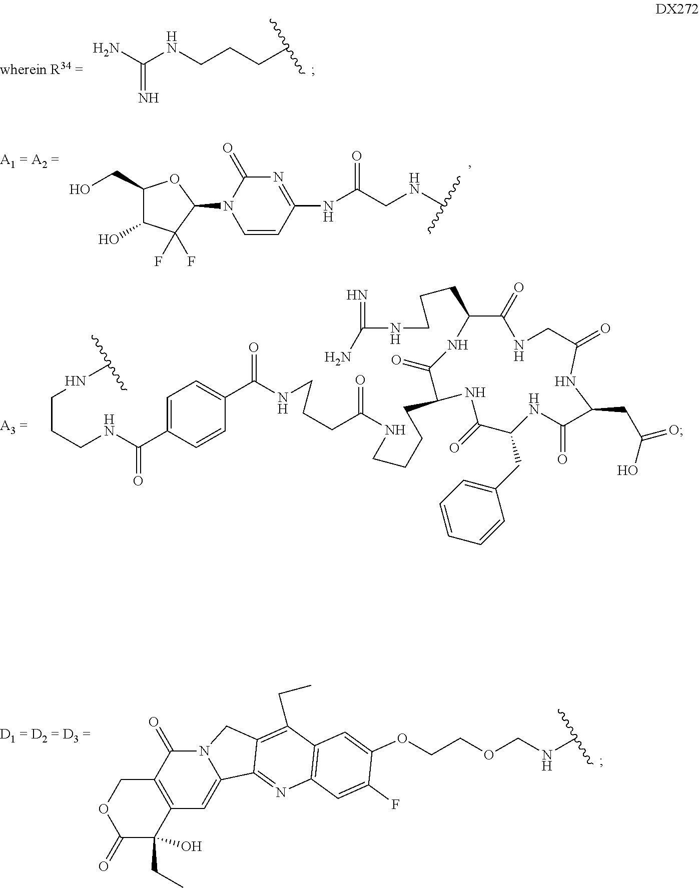 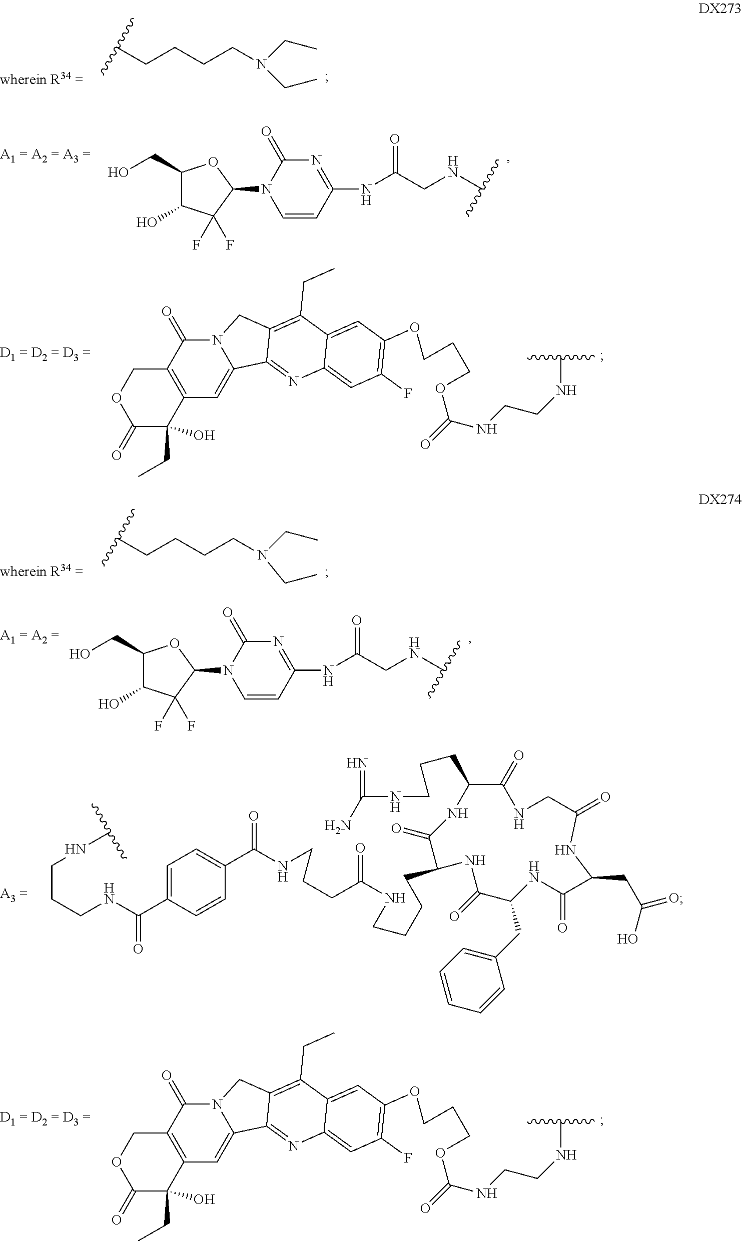 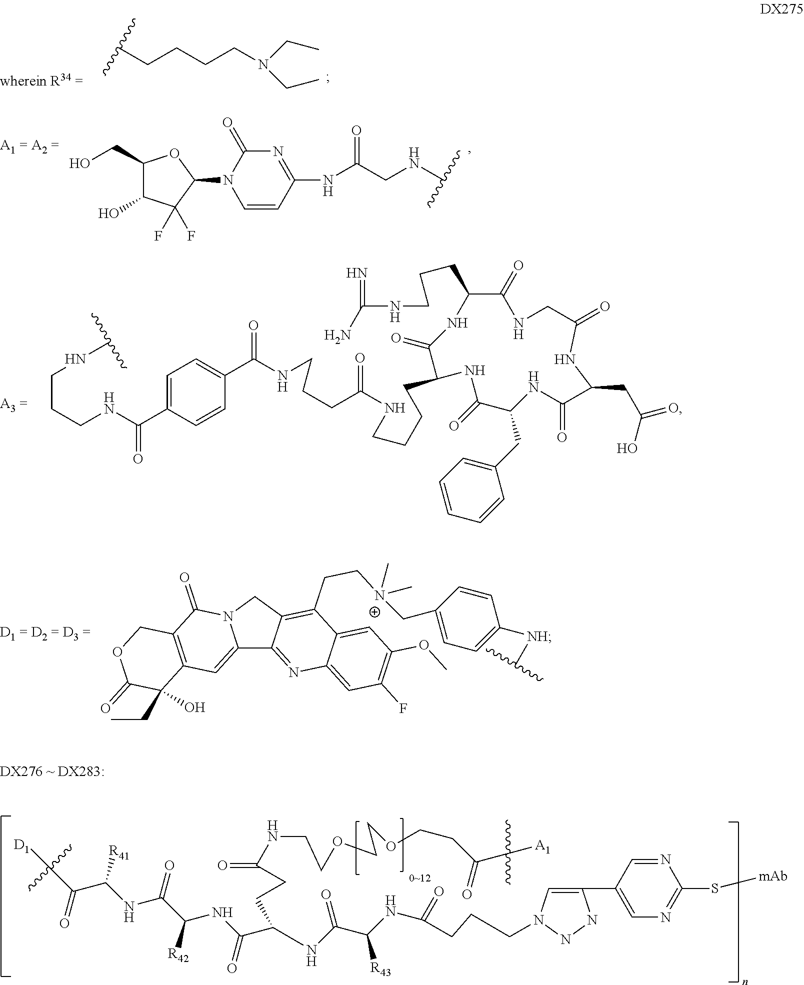 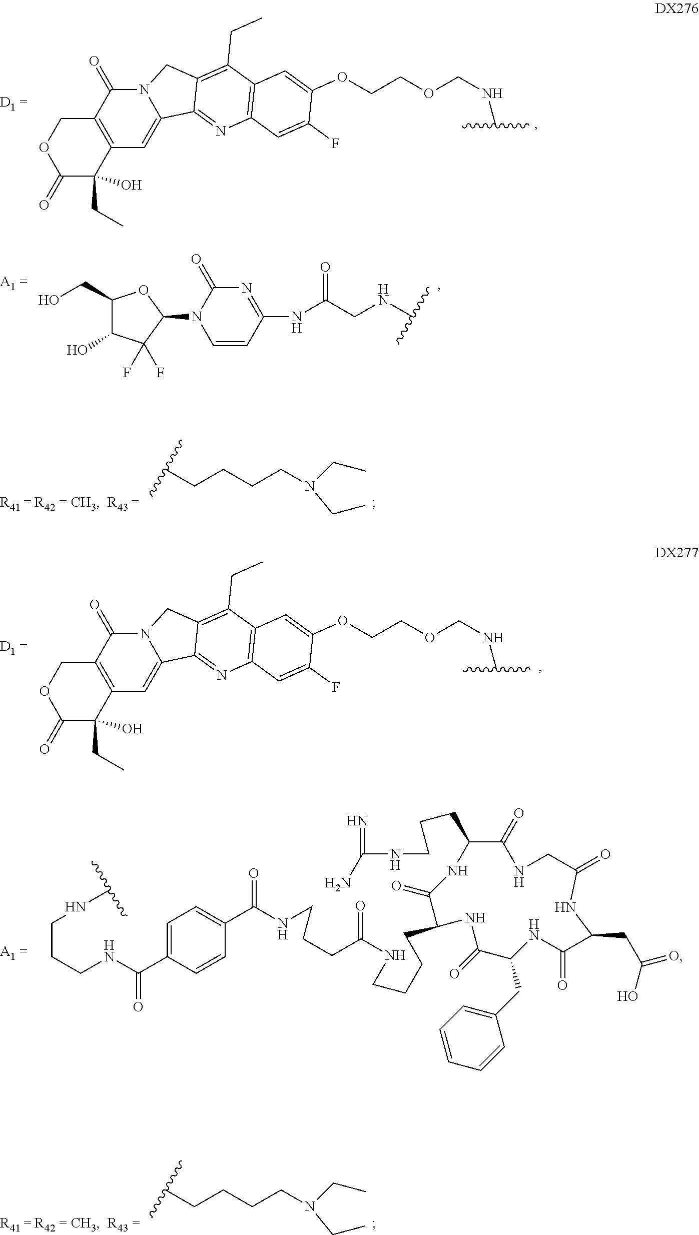 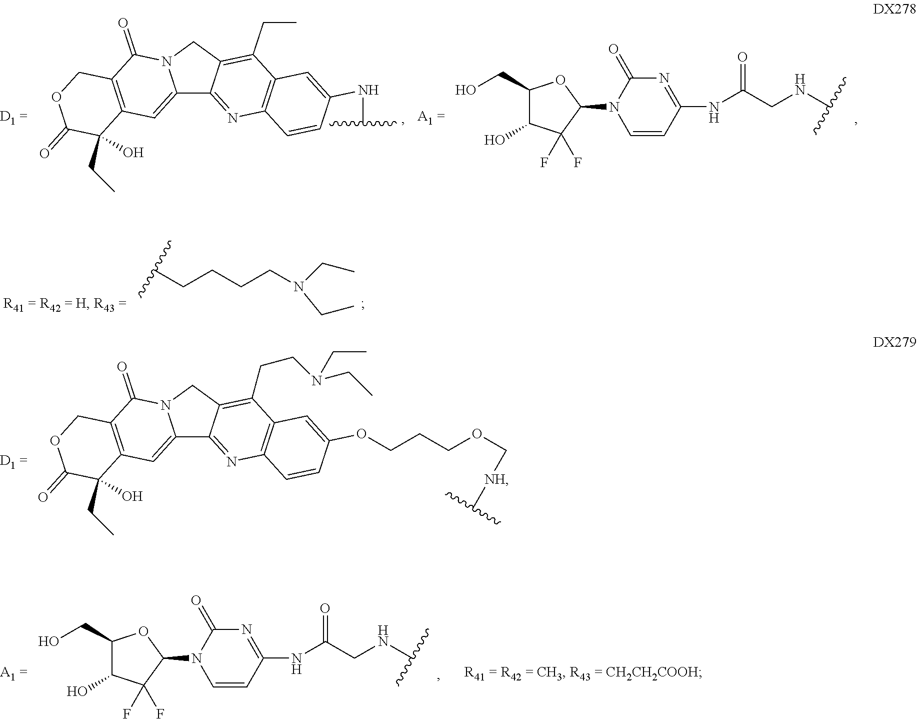 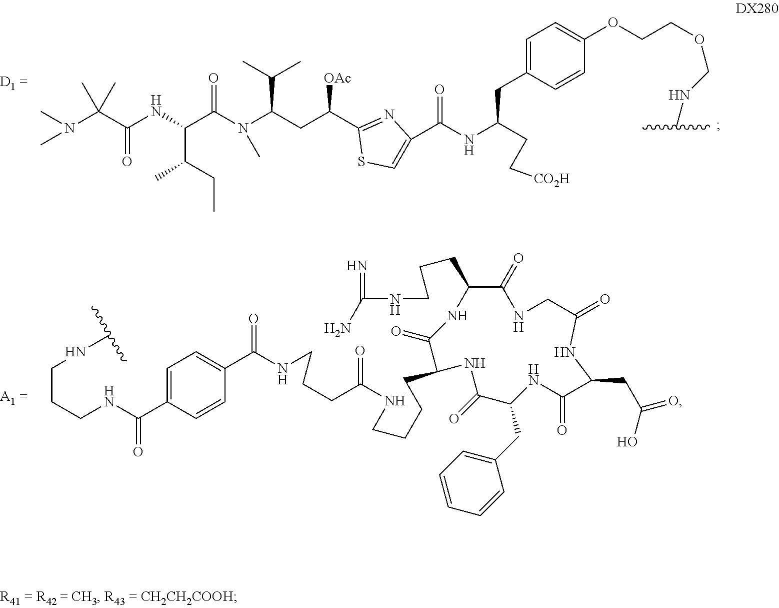 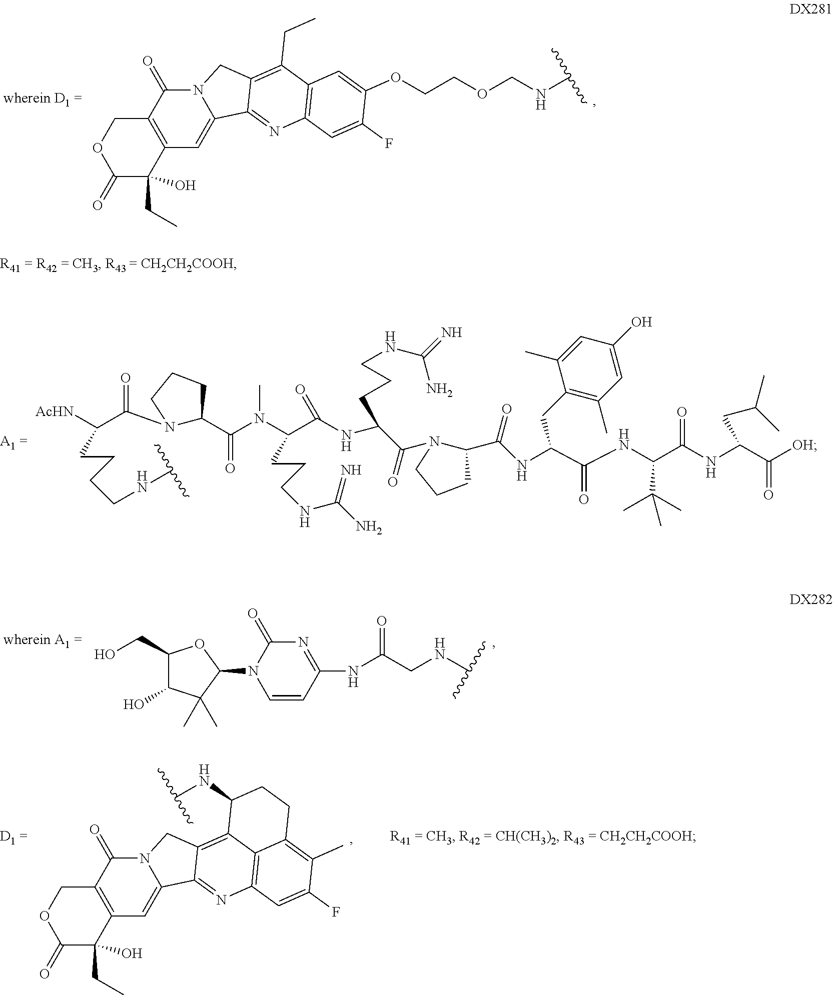 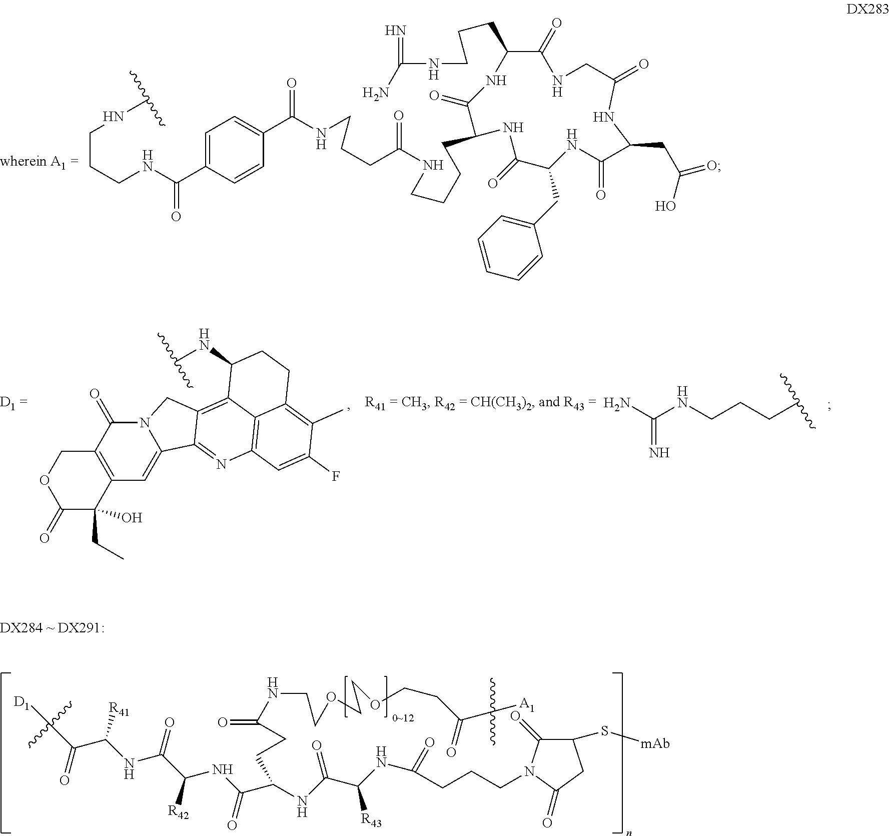 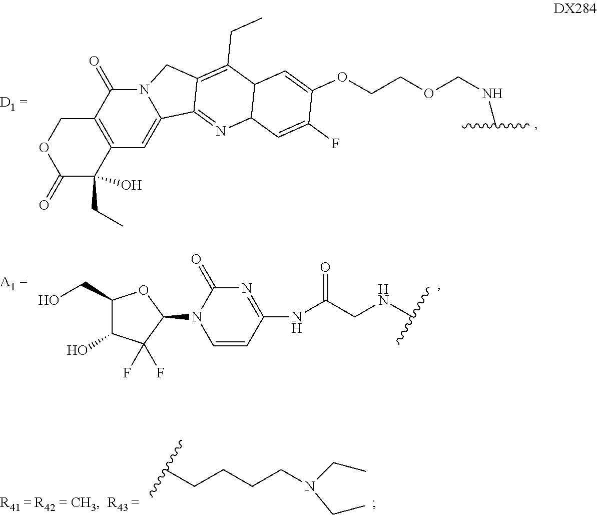 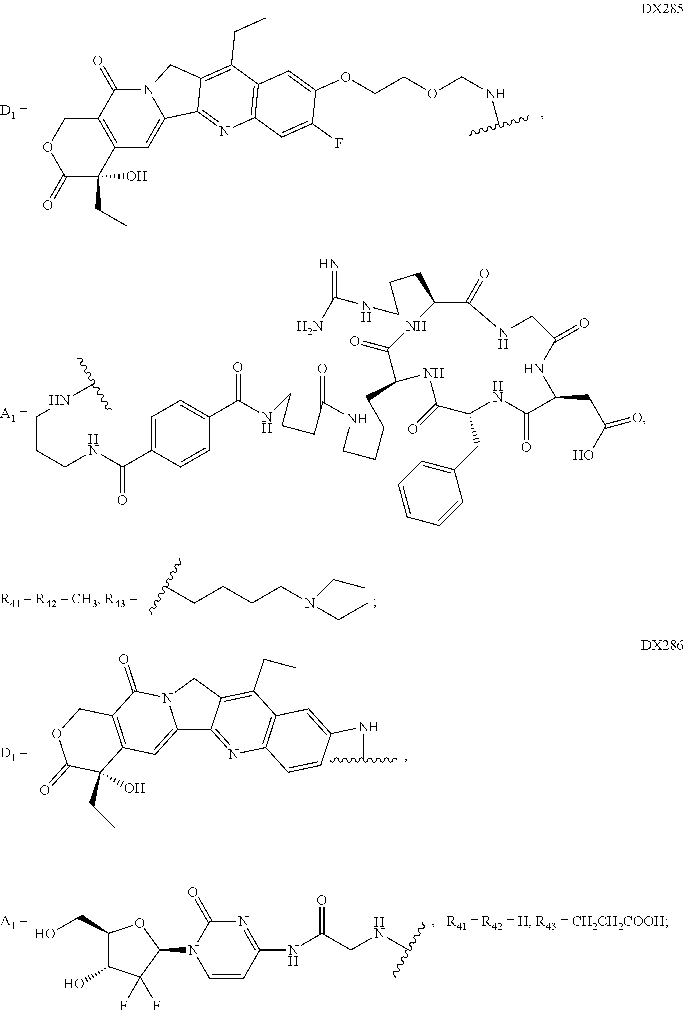 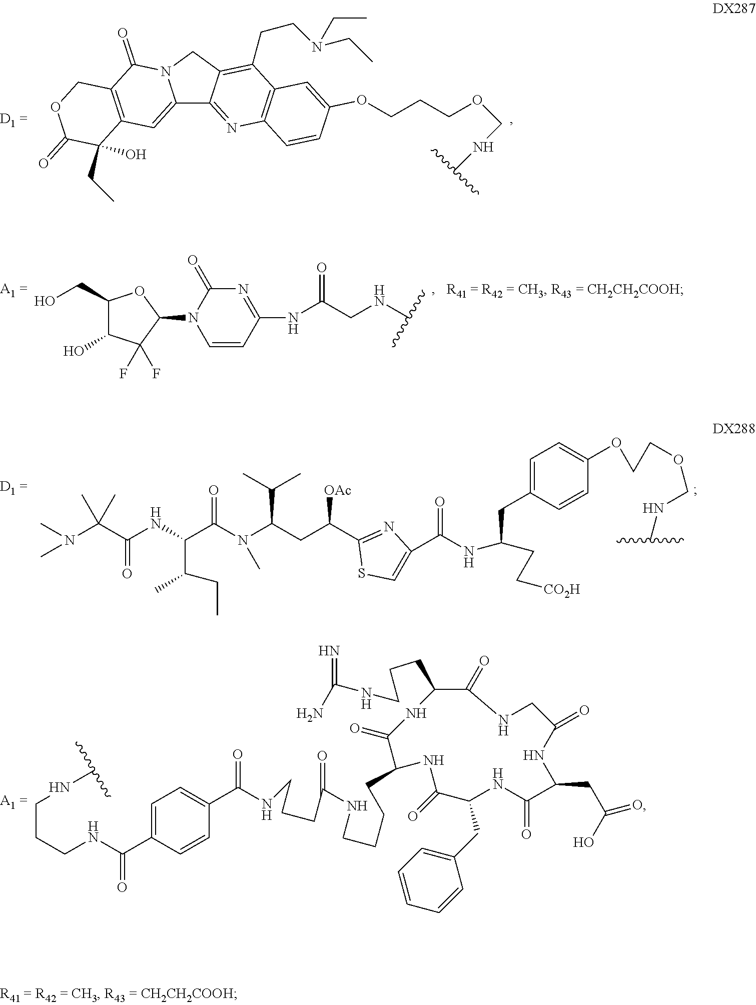 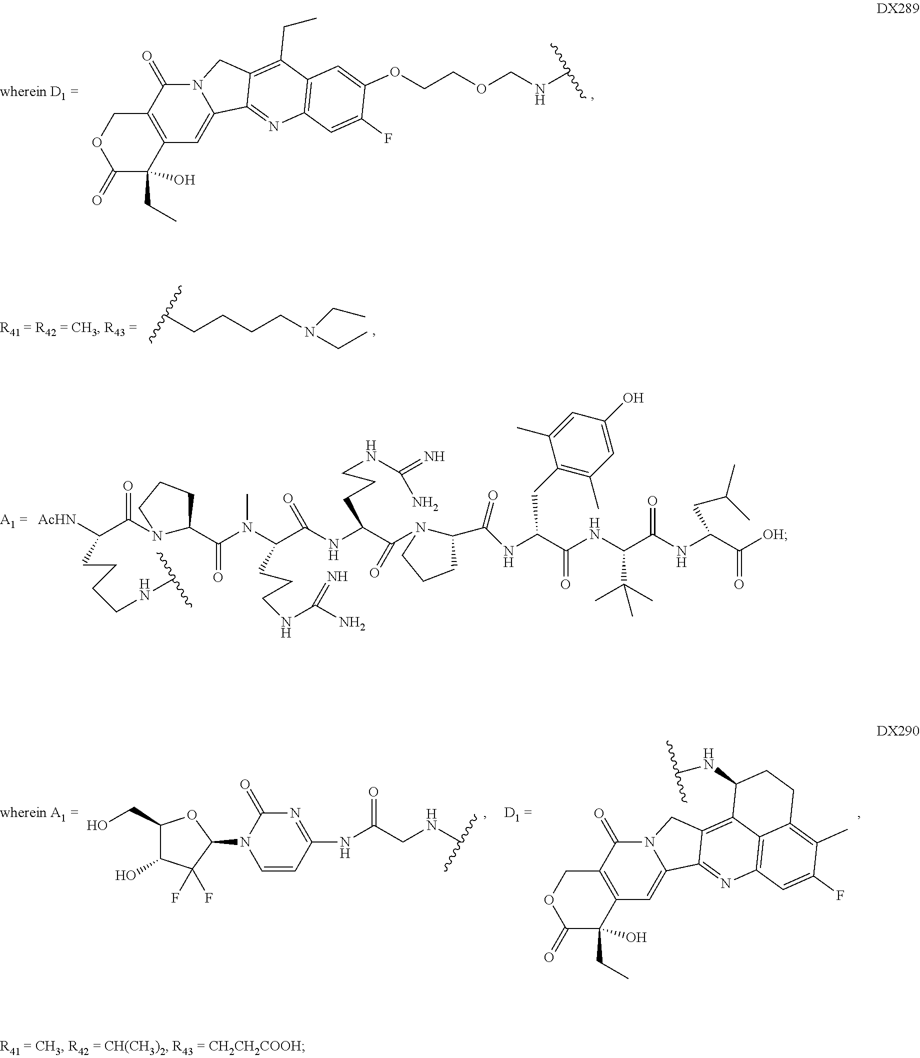 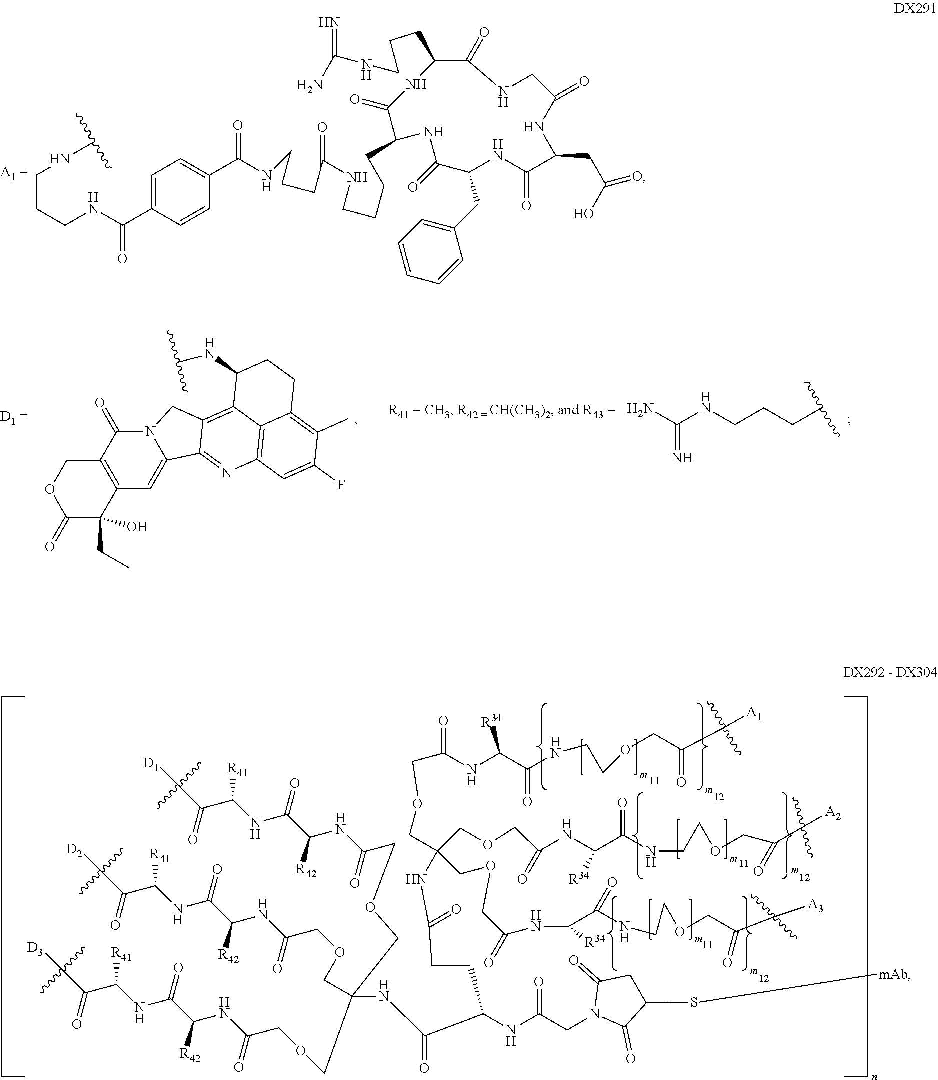
wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H4OH;
R³⁴ is H, CH₃, CH₂CH₃, C(O)H, and C(O)CH₃; D1, D₂, D3 and D4 are cytotoxic drug and A₁, A₂ and A₃ are small molecular ligands or drugs m₁₁ is a number of 0, 1, 2, 3, 4, 5, 6, 7, 8, 9 and m₁₂ is a number of 0, 1, 2, 3 and 4; when number is 0 for the brackets, it means the structure in the brackets can be omitted (crossed out), 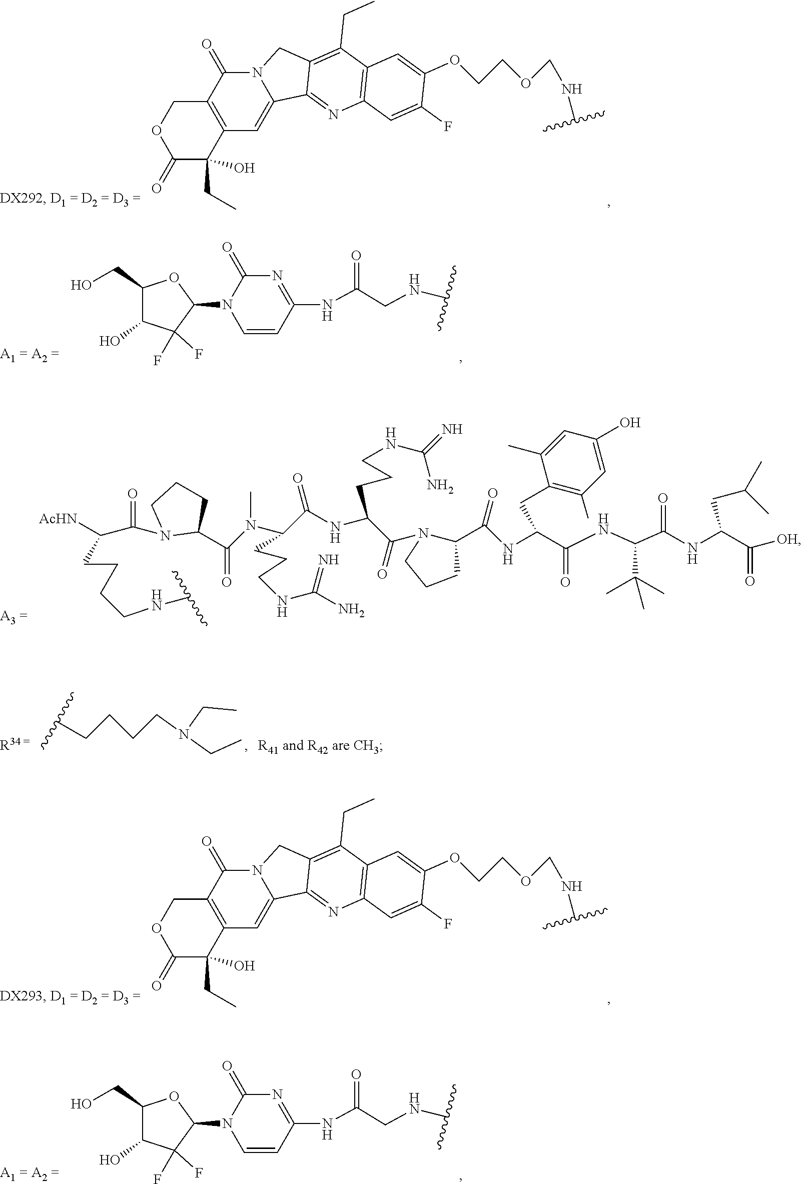 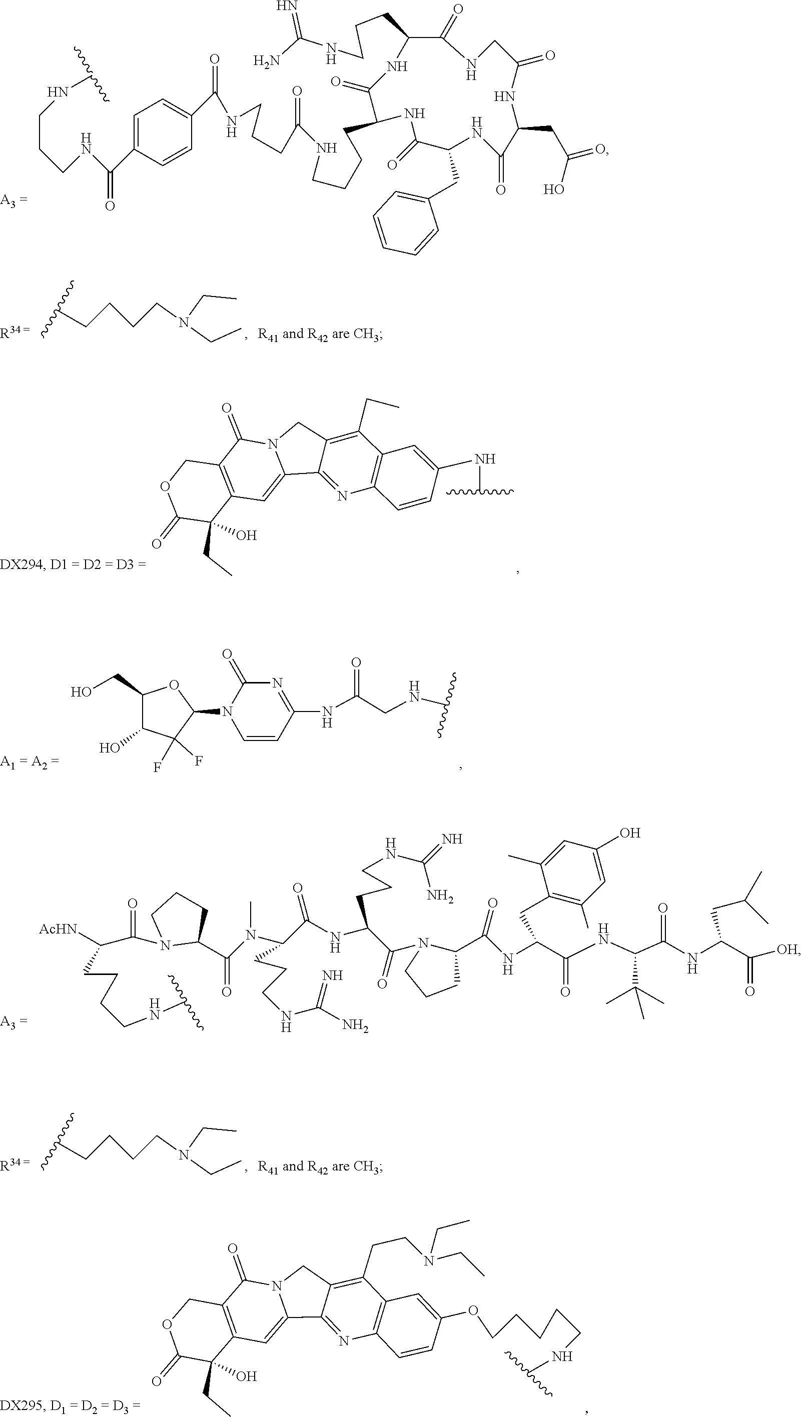 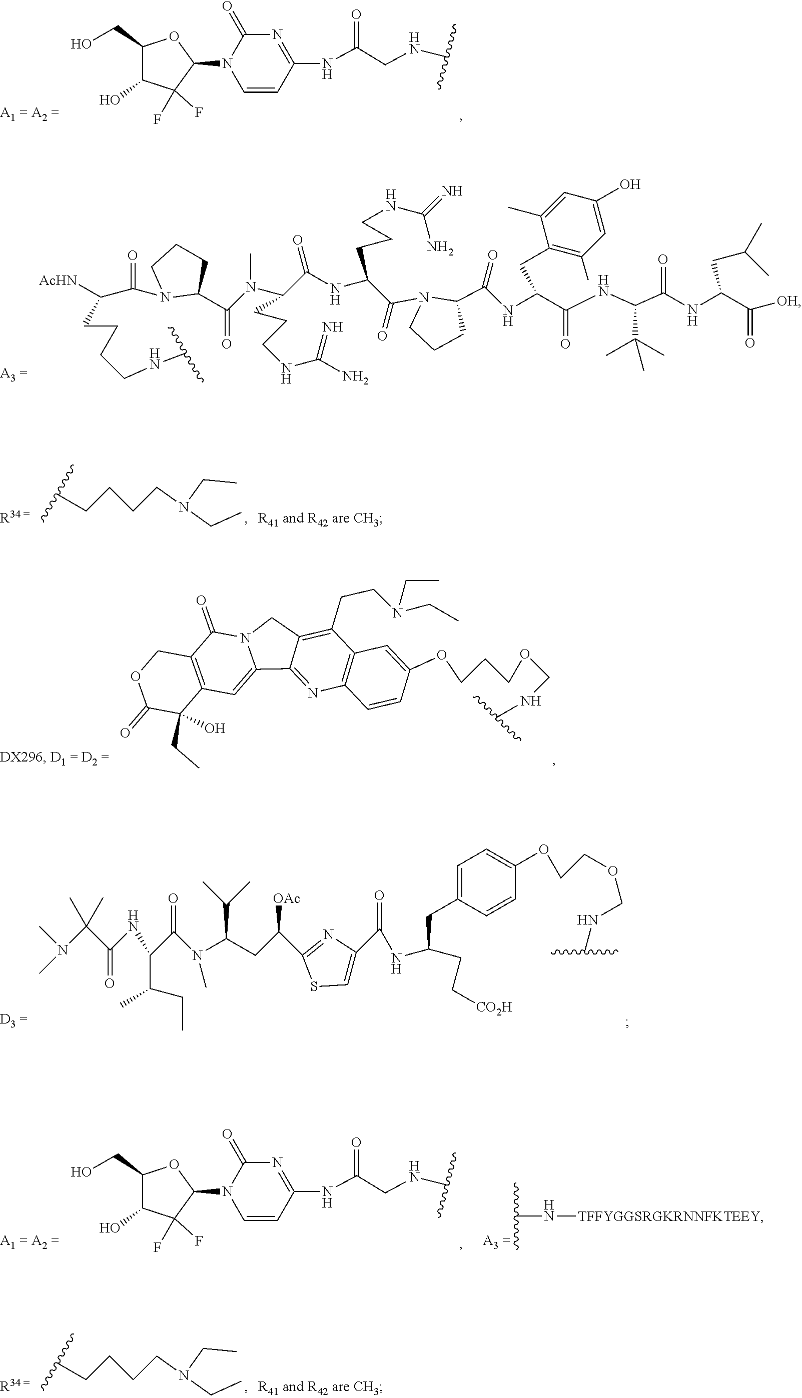 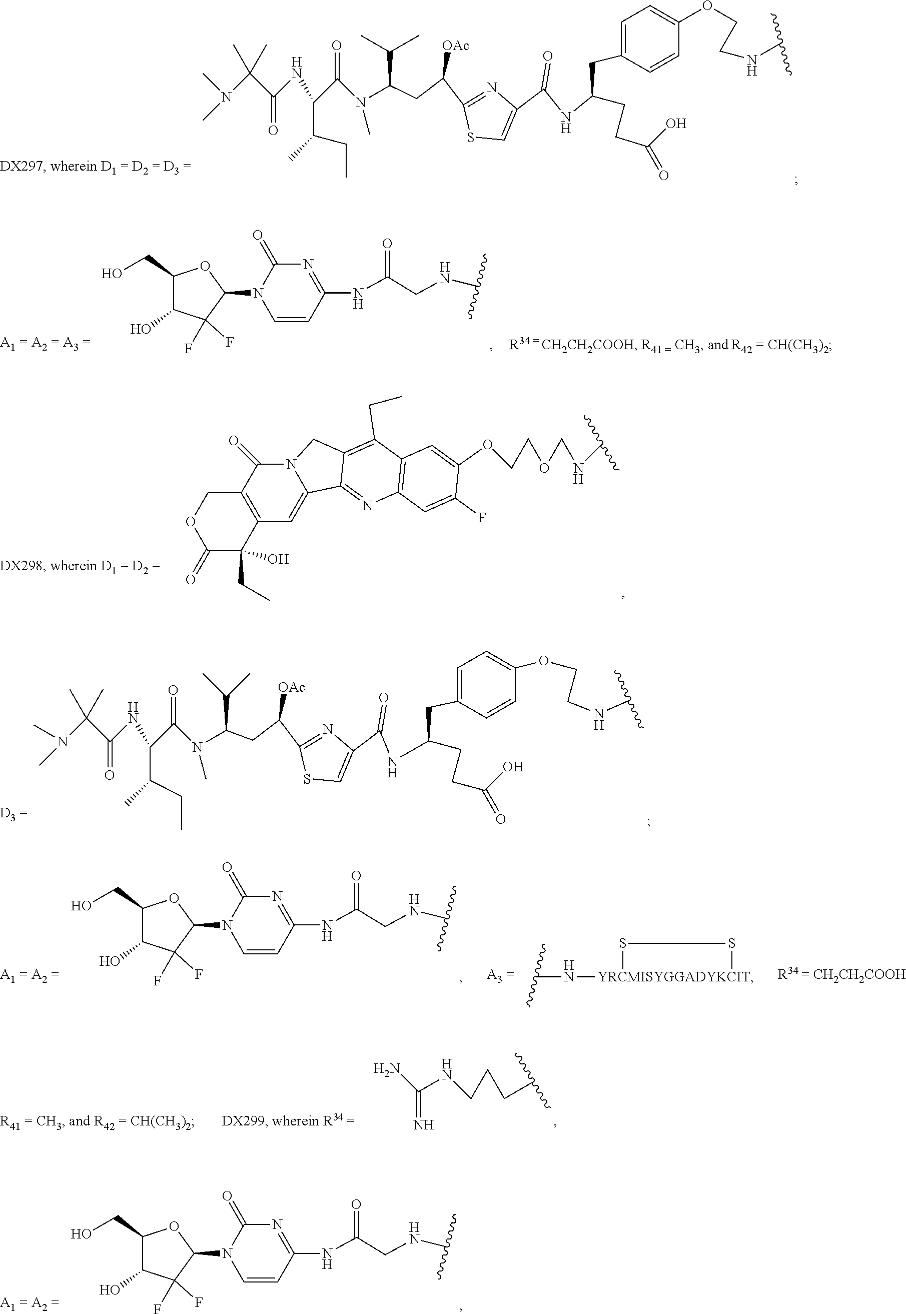 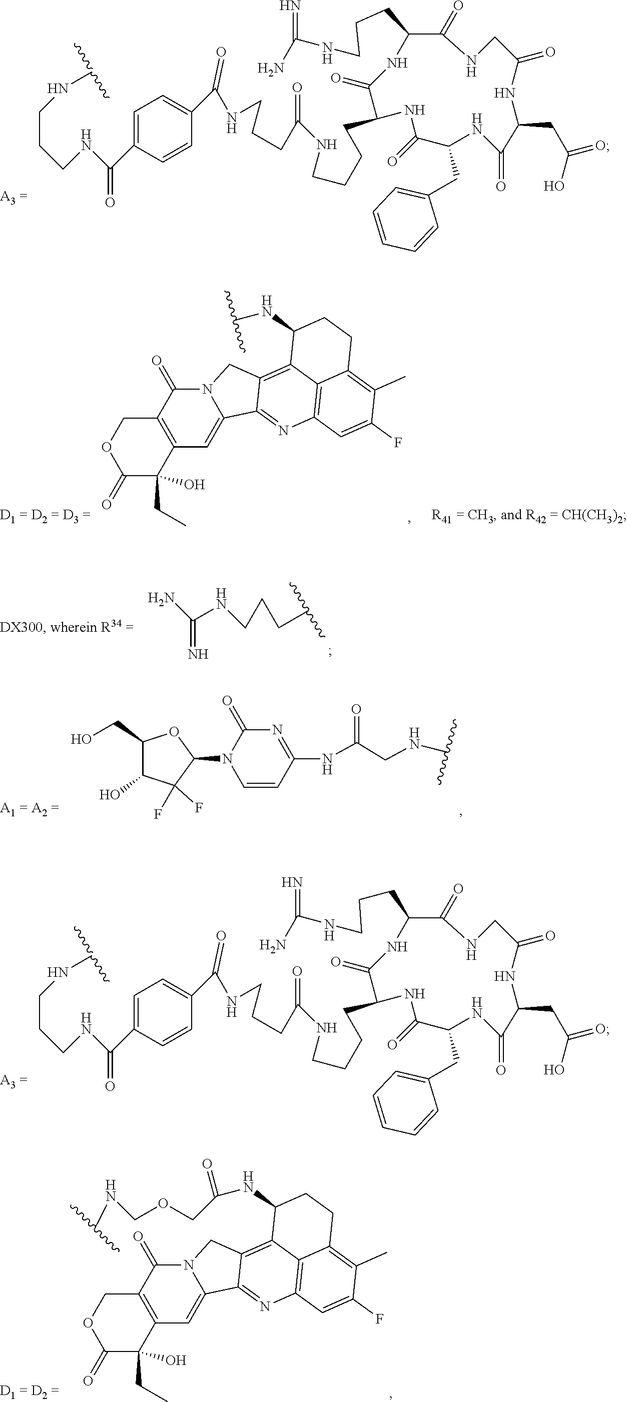 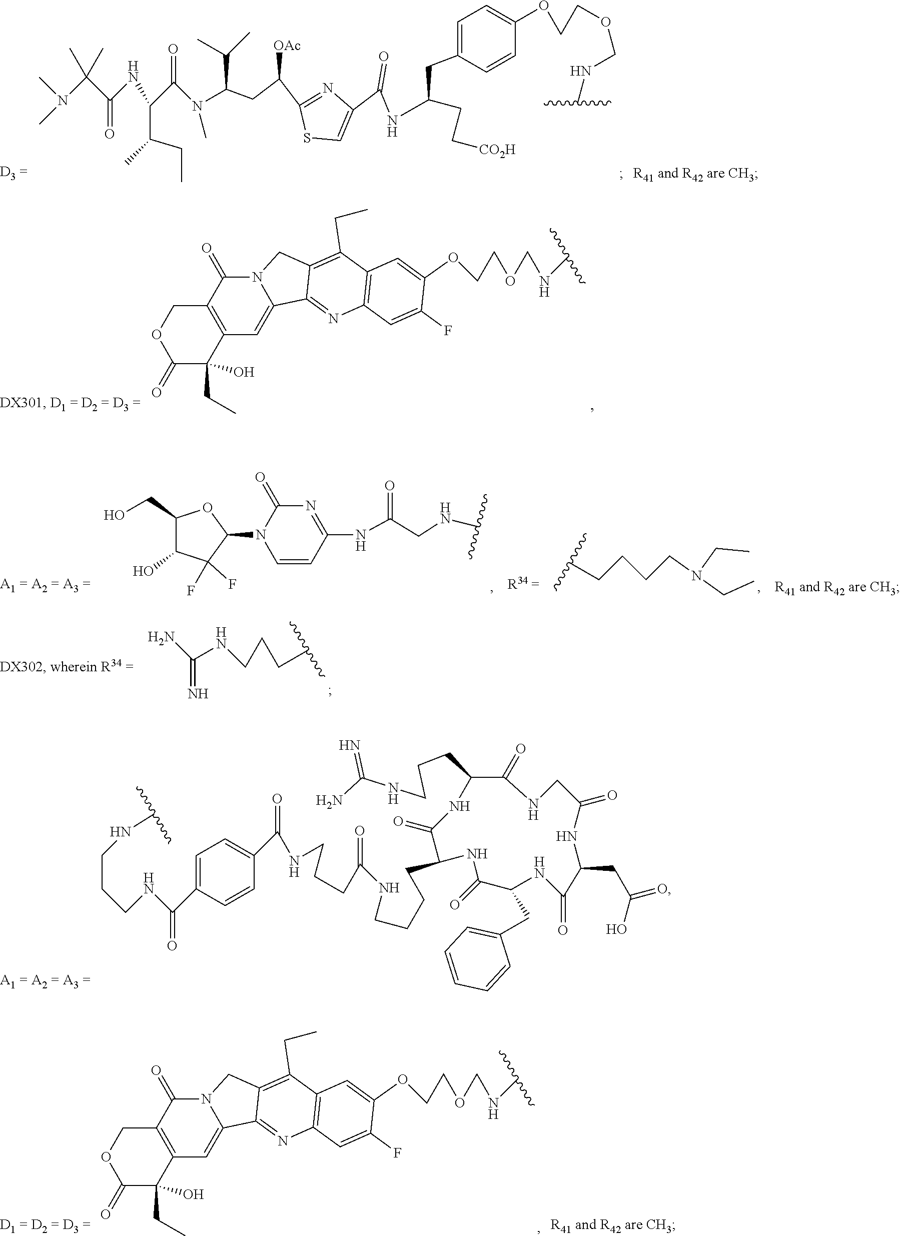 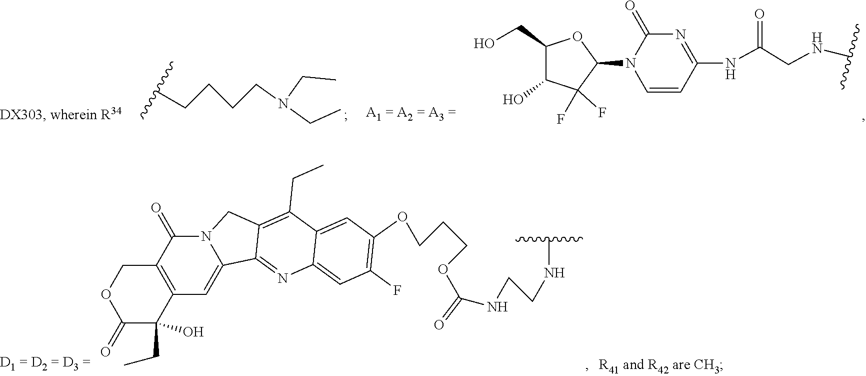 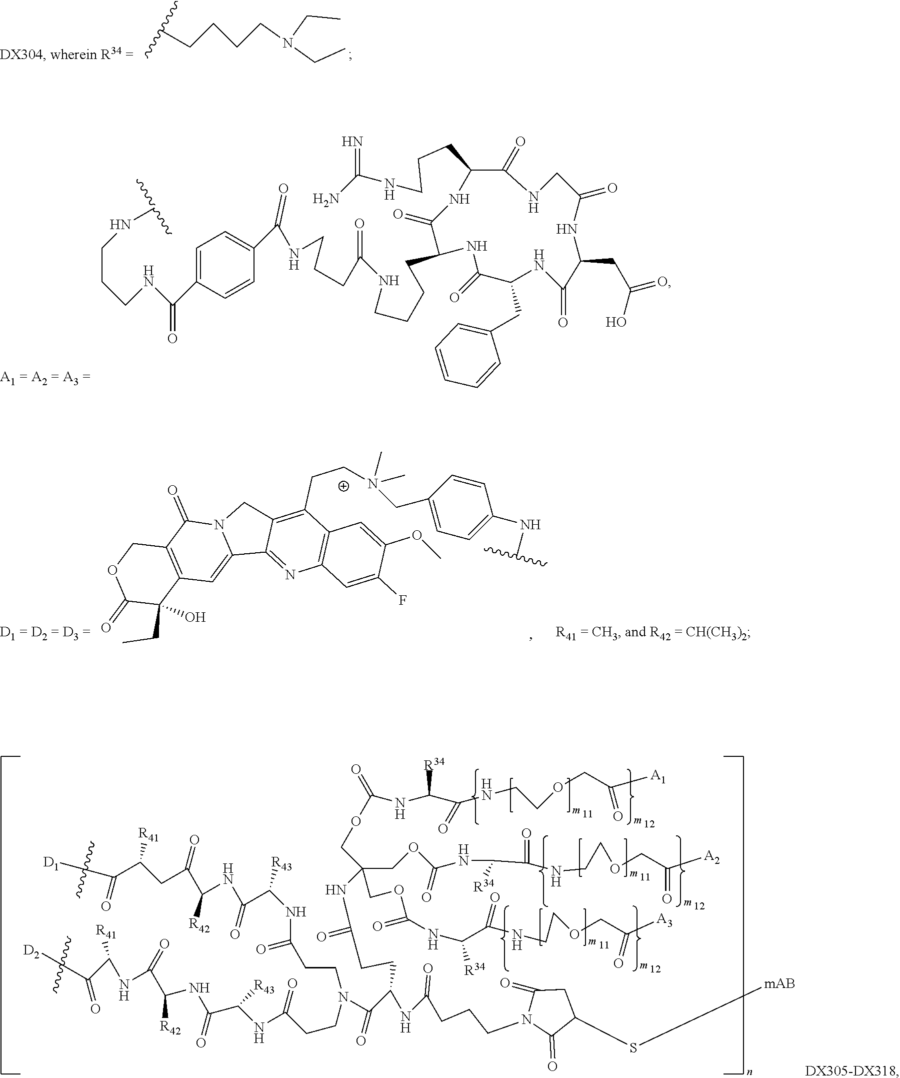
wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂, CH(CH, CH(CHCCH₃(C₂CH₂CH₃, CH₂CH₂S(O₂)CH₃, CH₂SH, CH₂SO₃H, CH₂C₆H₅, and CH₂C₆H4OH;
R³⁴ is H, CH₃, CH₂CH₃, C(O)H, and C(O)CH₃; D1, D₂, D3 and D4 are cytotoxic drug and A₁, A₂ and A₃ are small molecular ligands or drugs m₁₁ is a number of 0, 1, 2, 3, 4, 5, 6, 7, 8, 9 and m₁₂ is a number of 0, 1, 2, 3 and 4; when number is 0, it means the structure in the brackets can be omitted (crossed out). 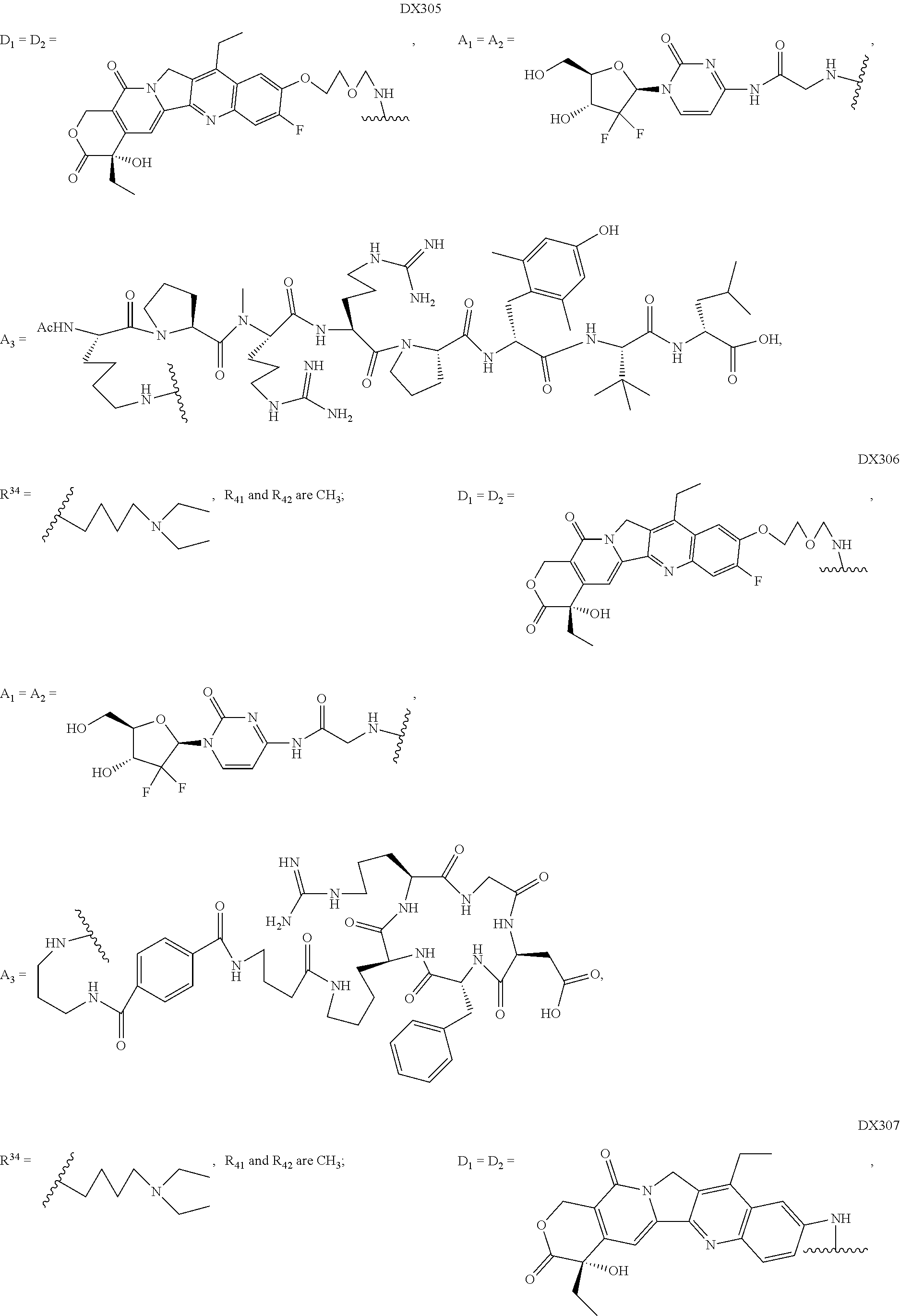 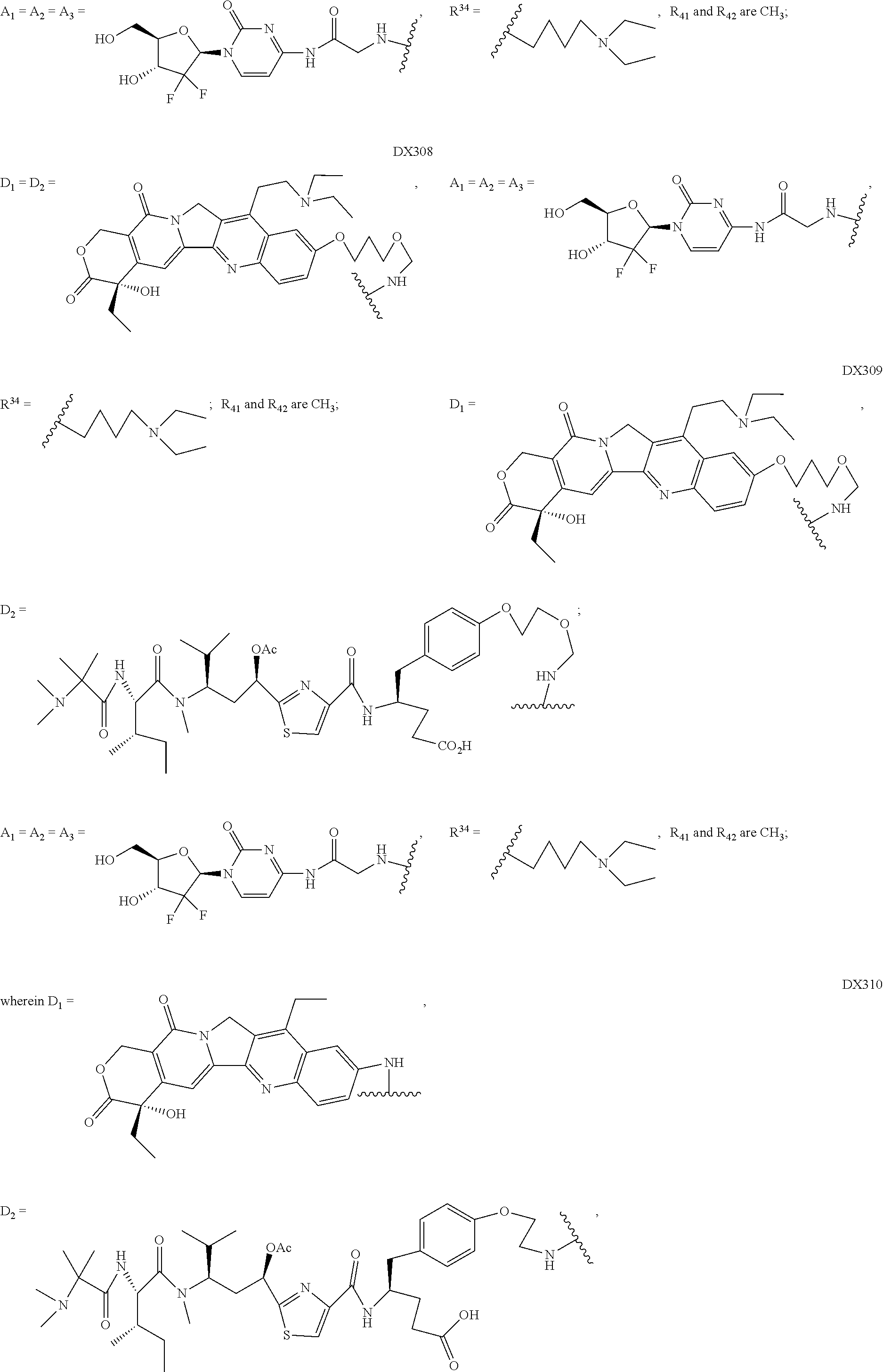 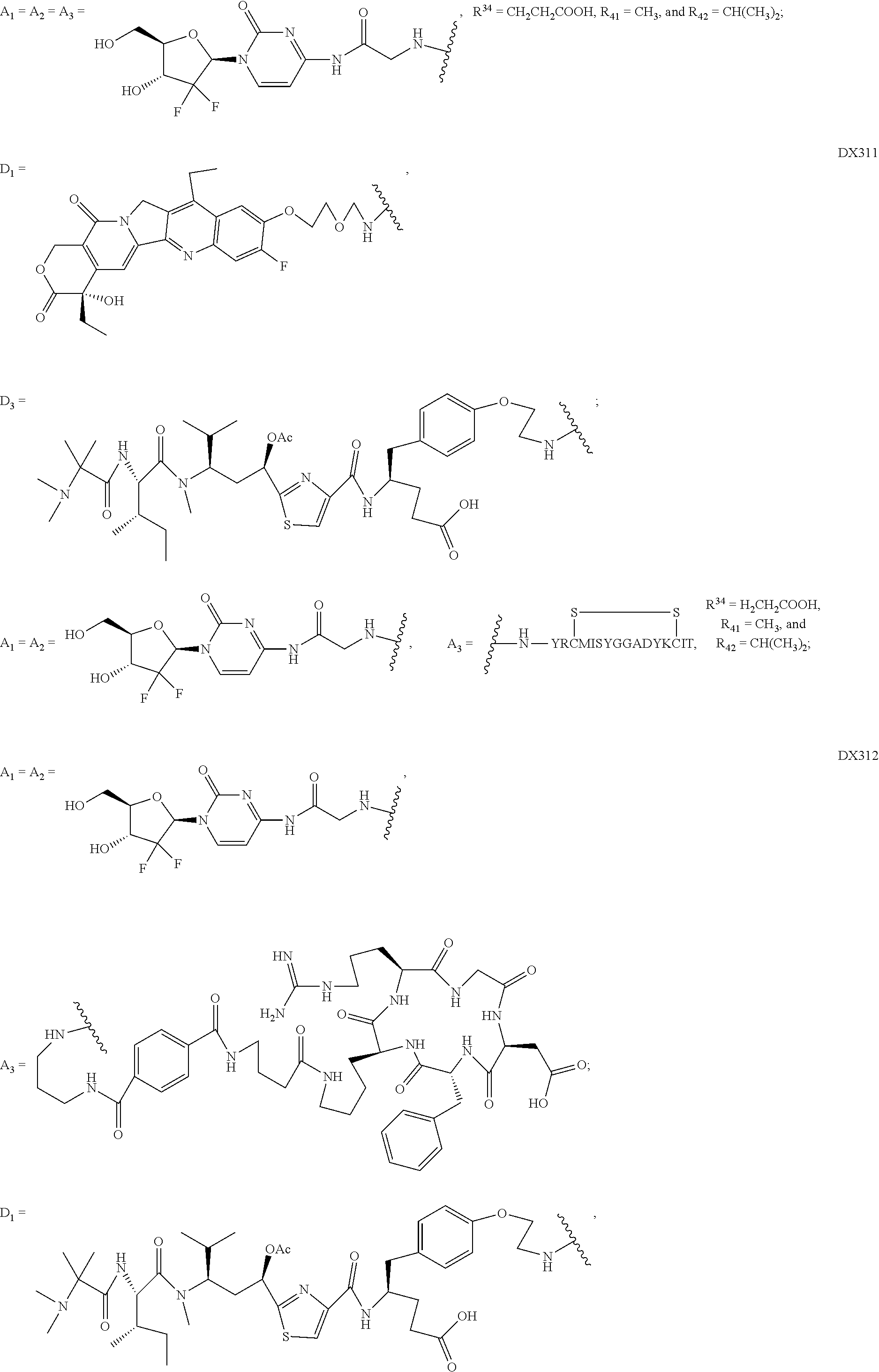 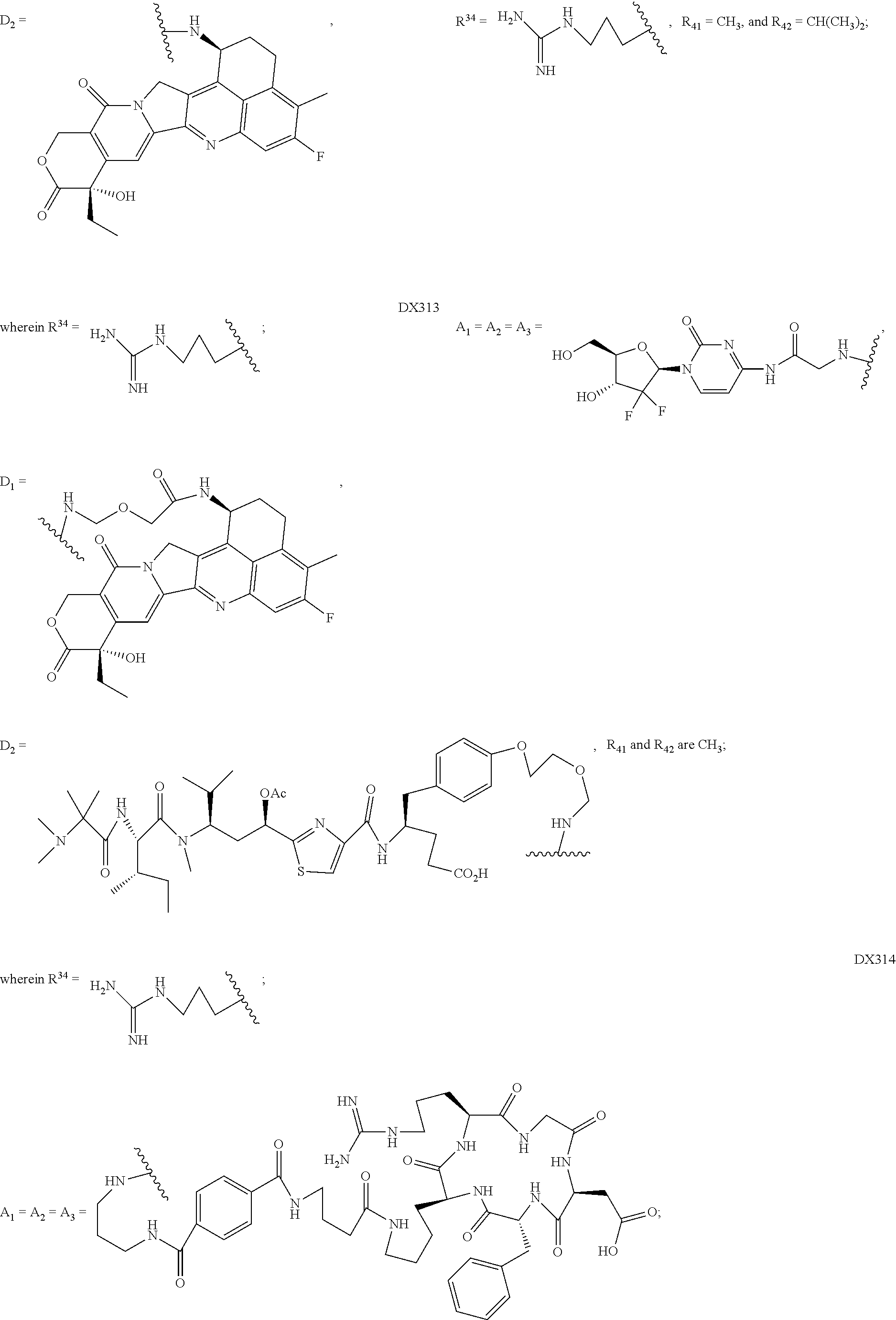 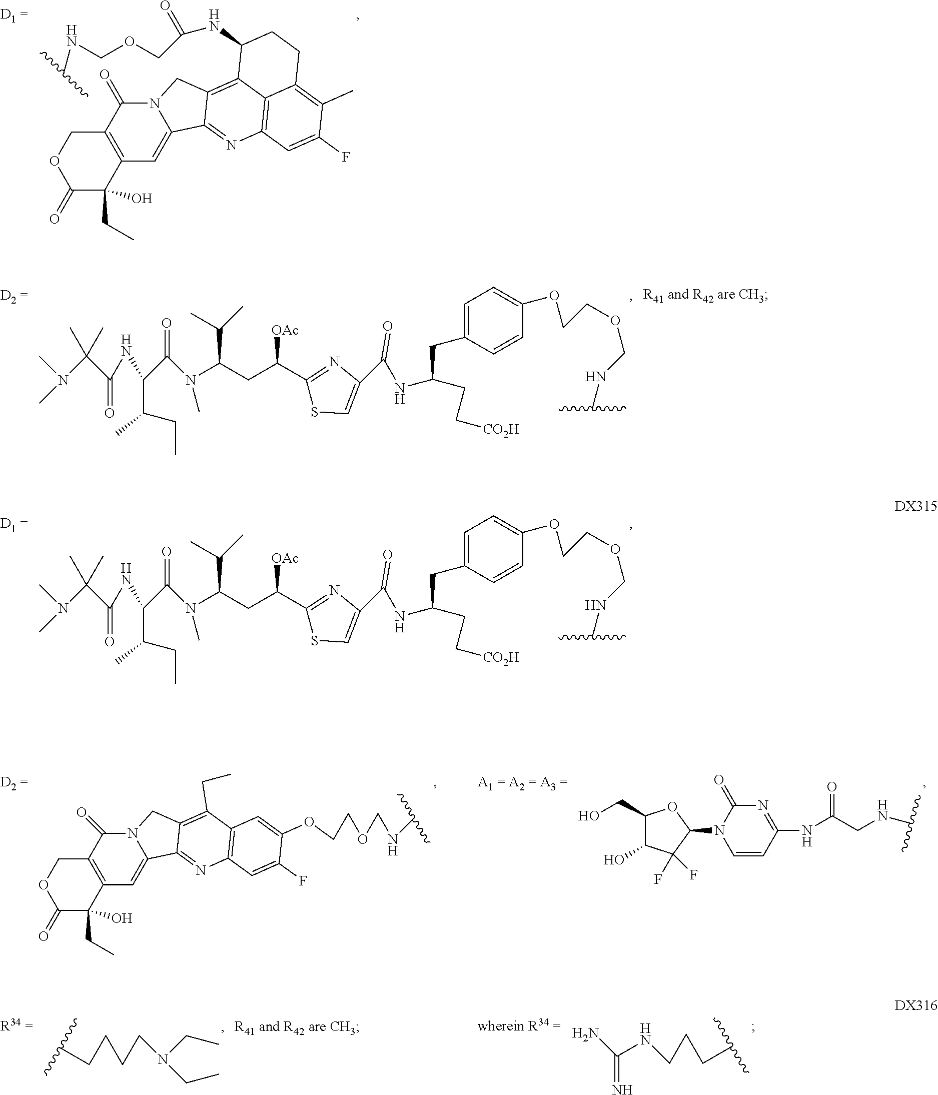 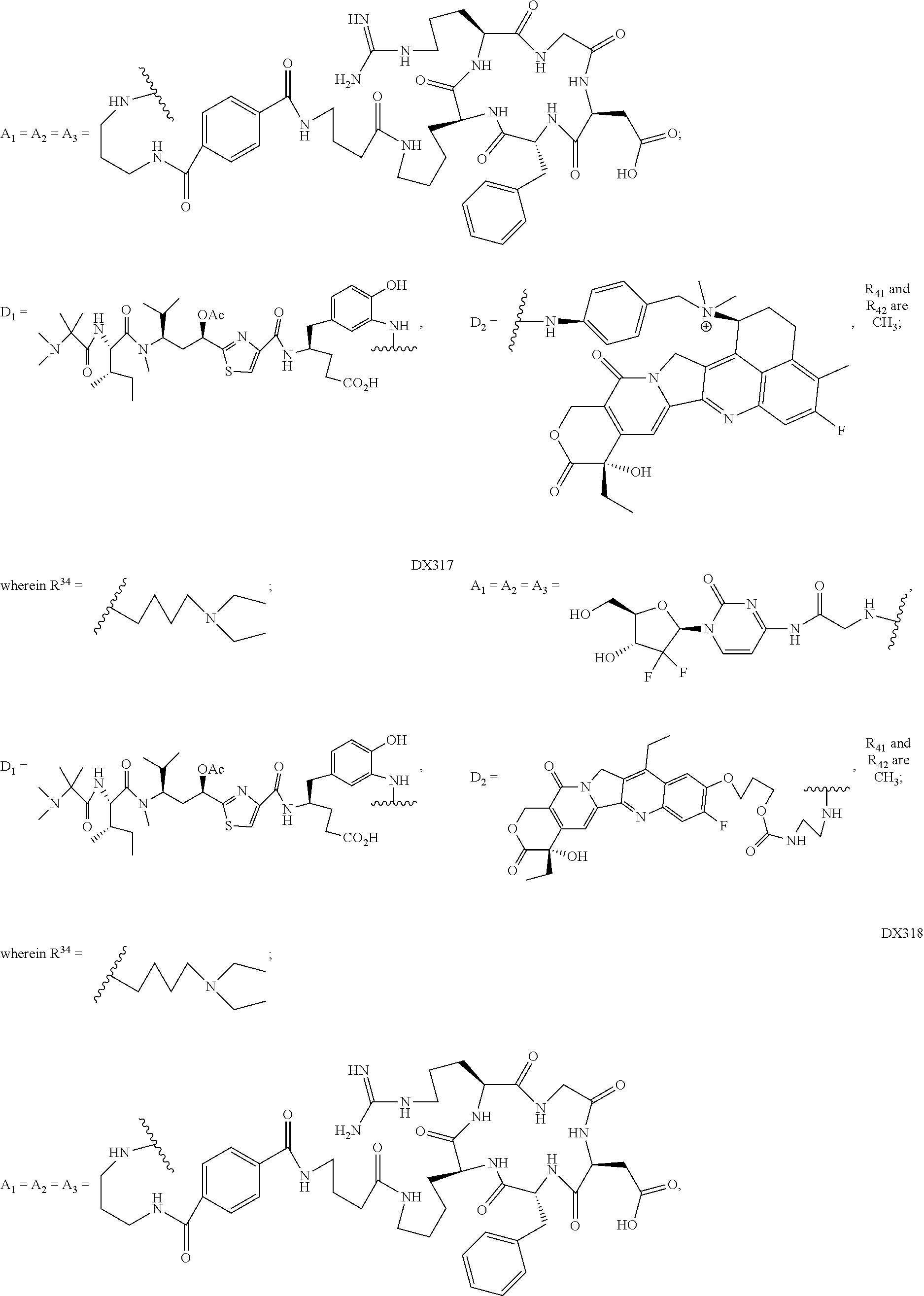 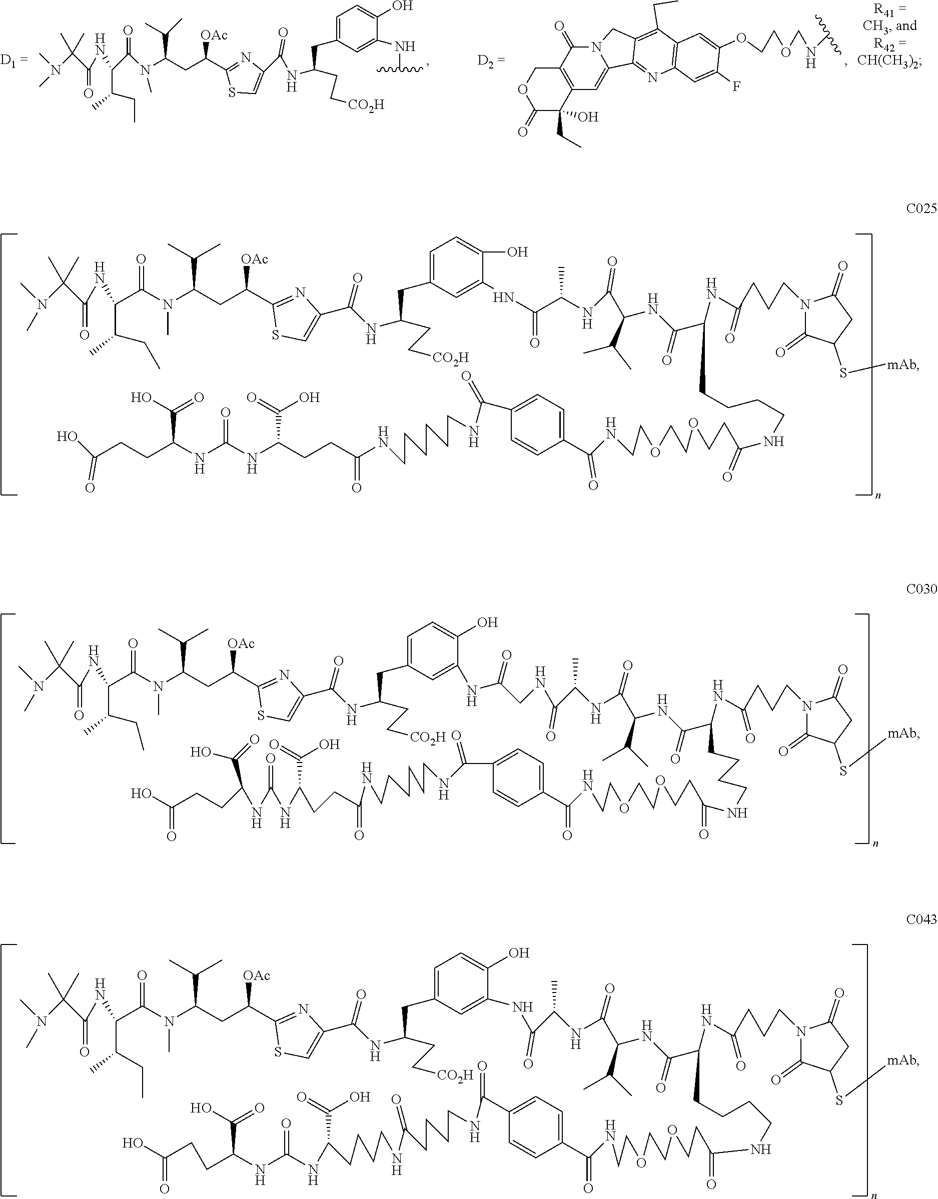 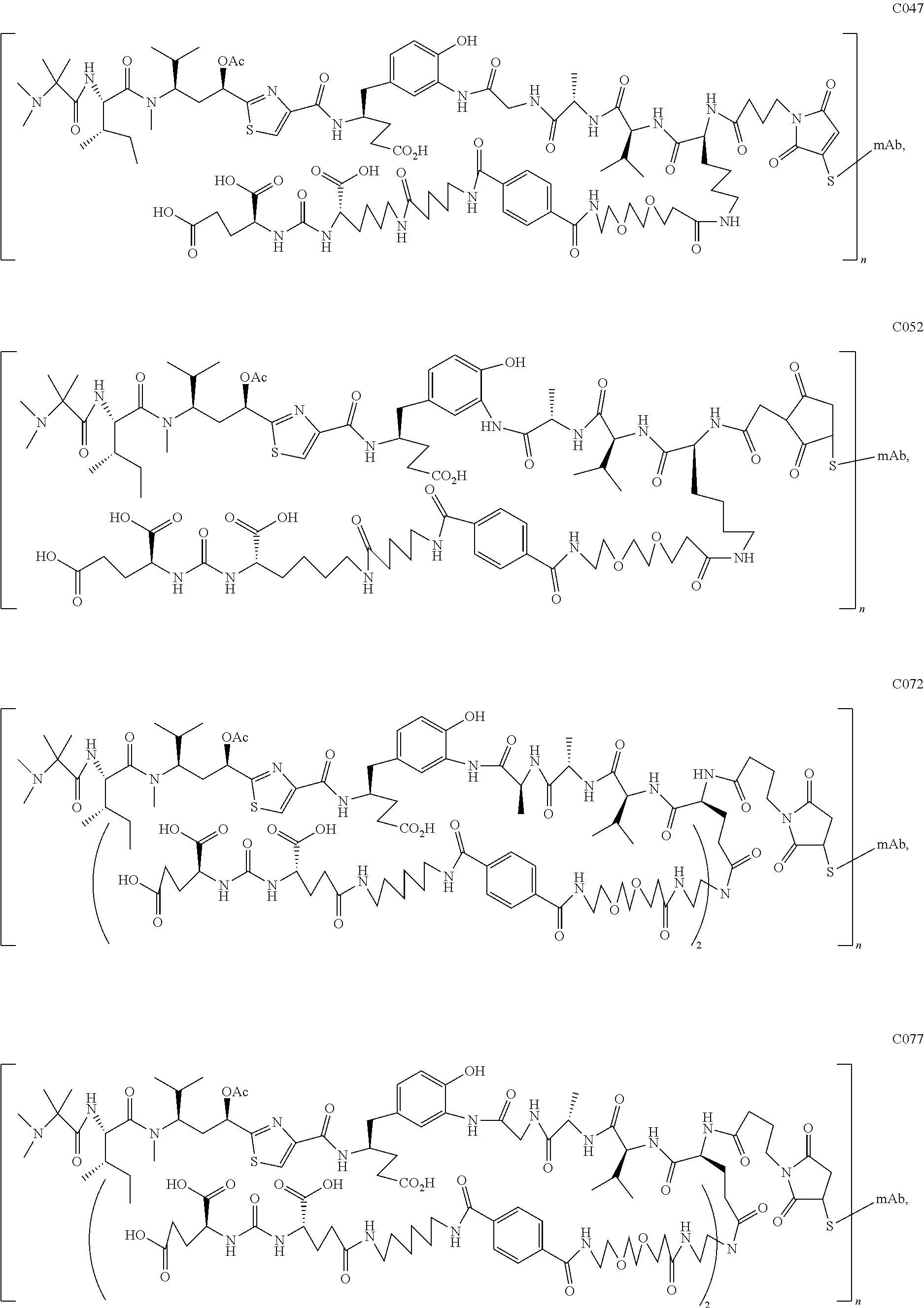 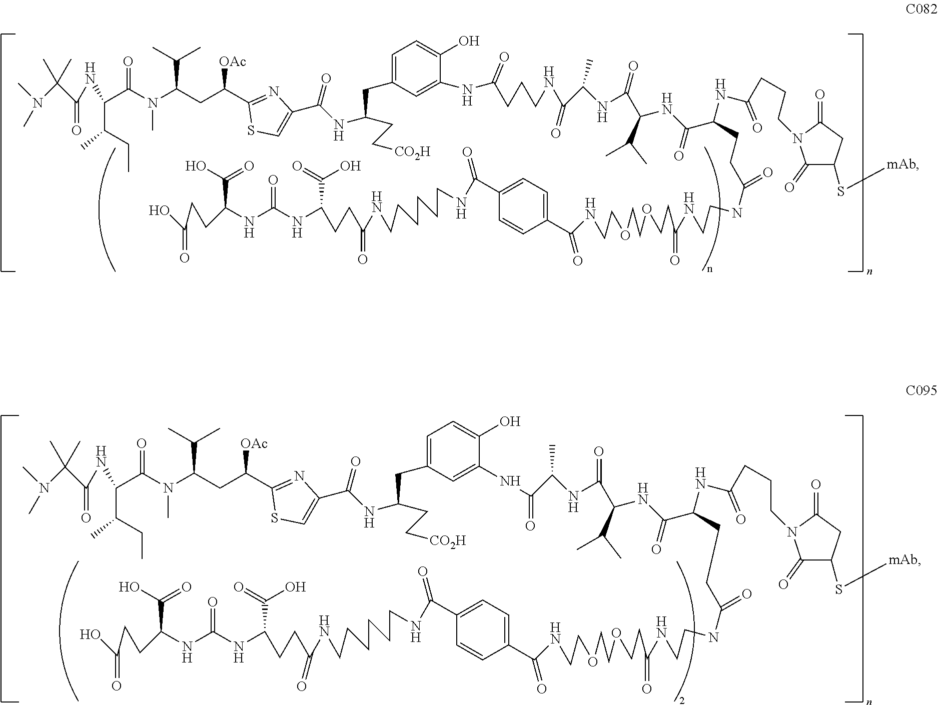 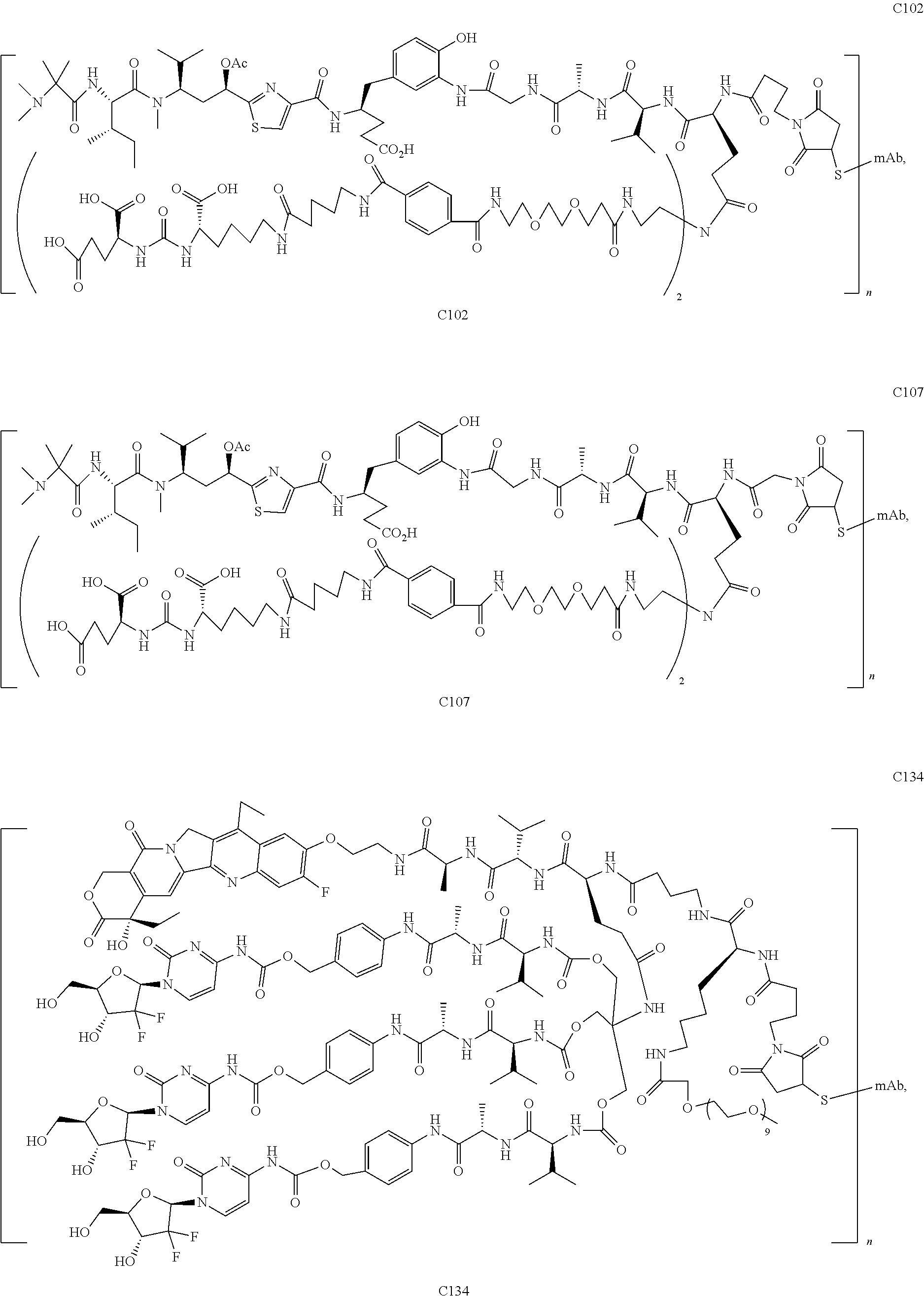 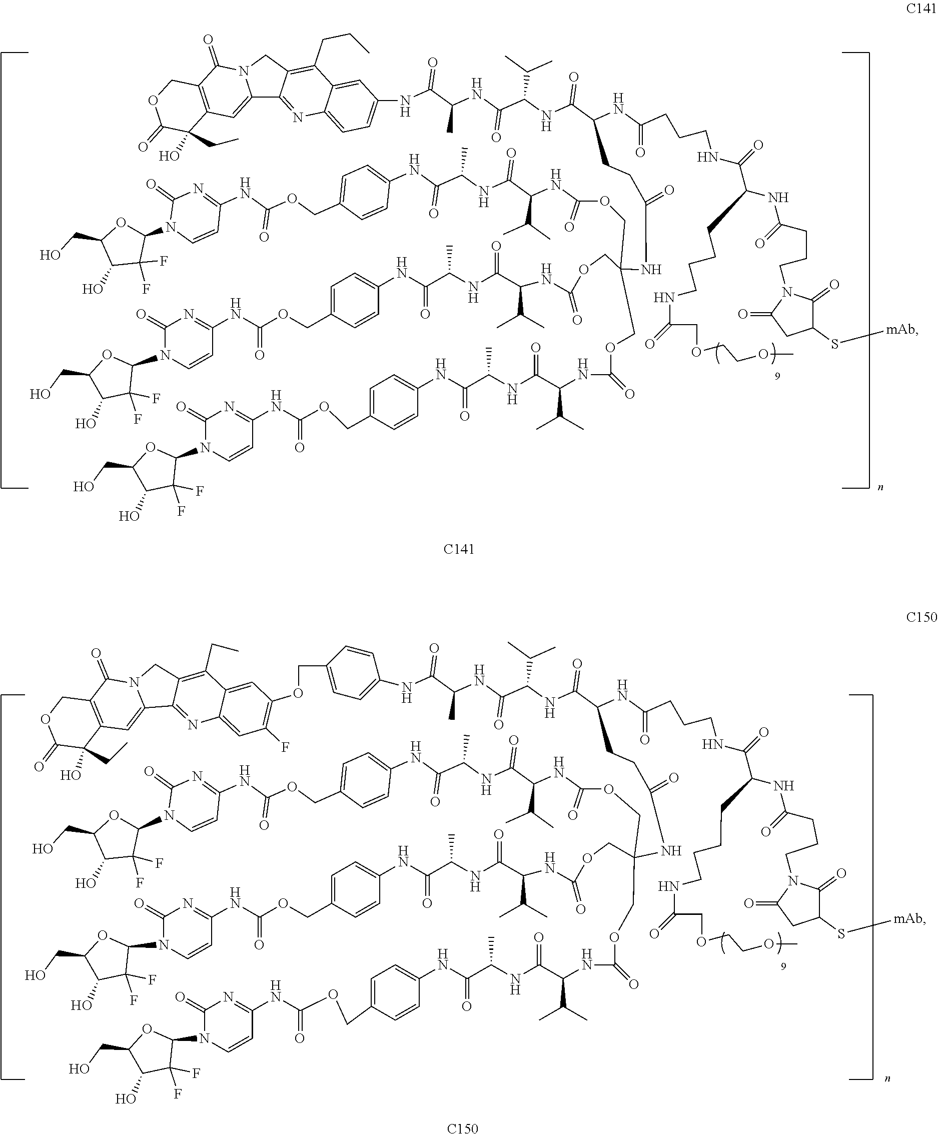 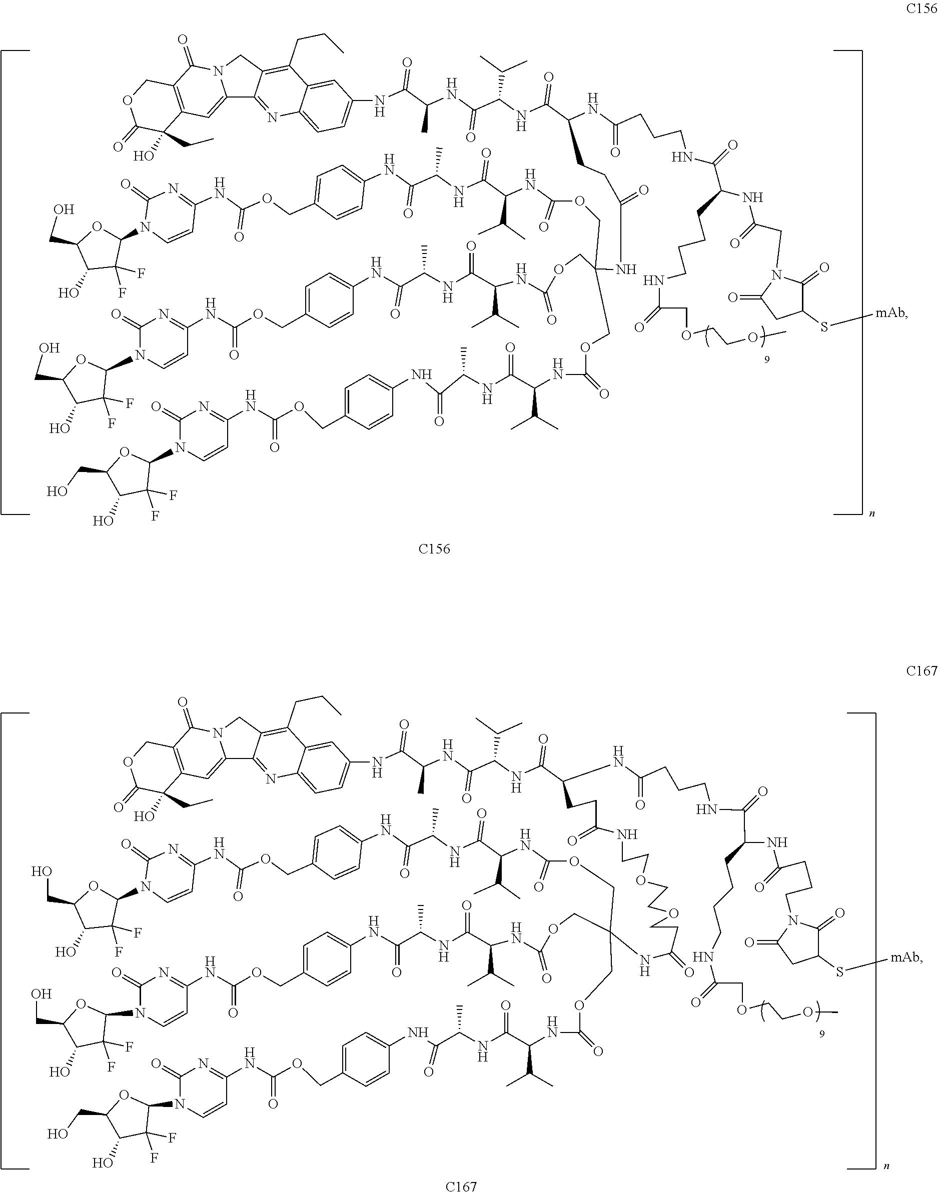 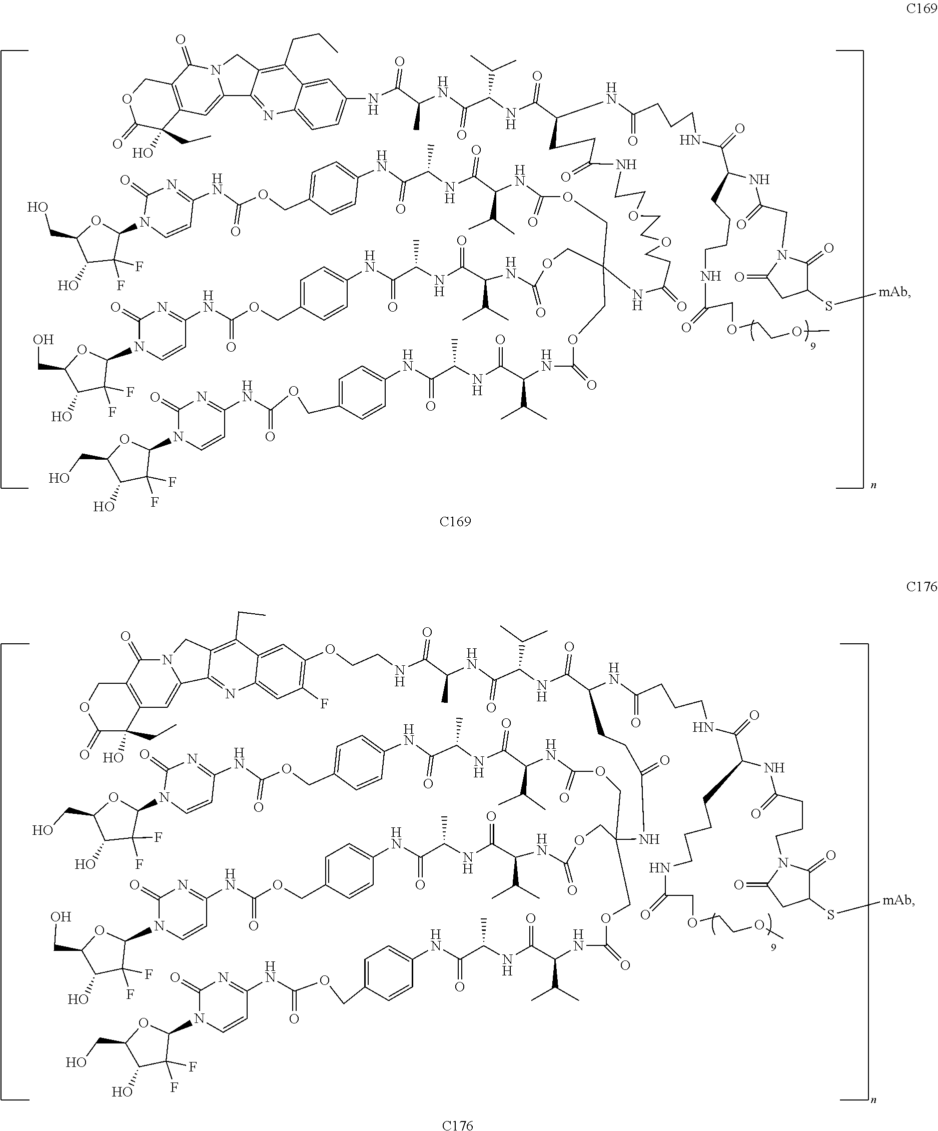 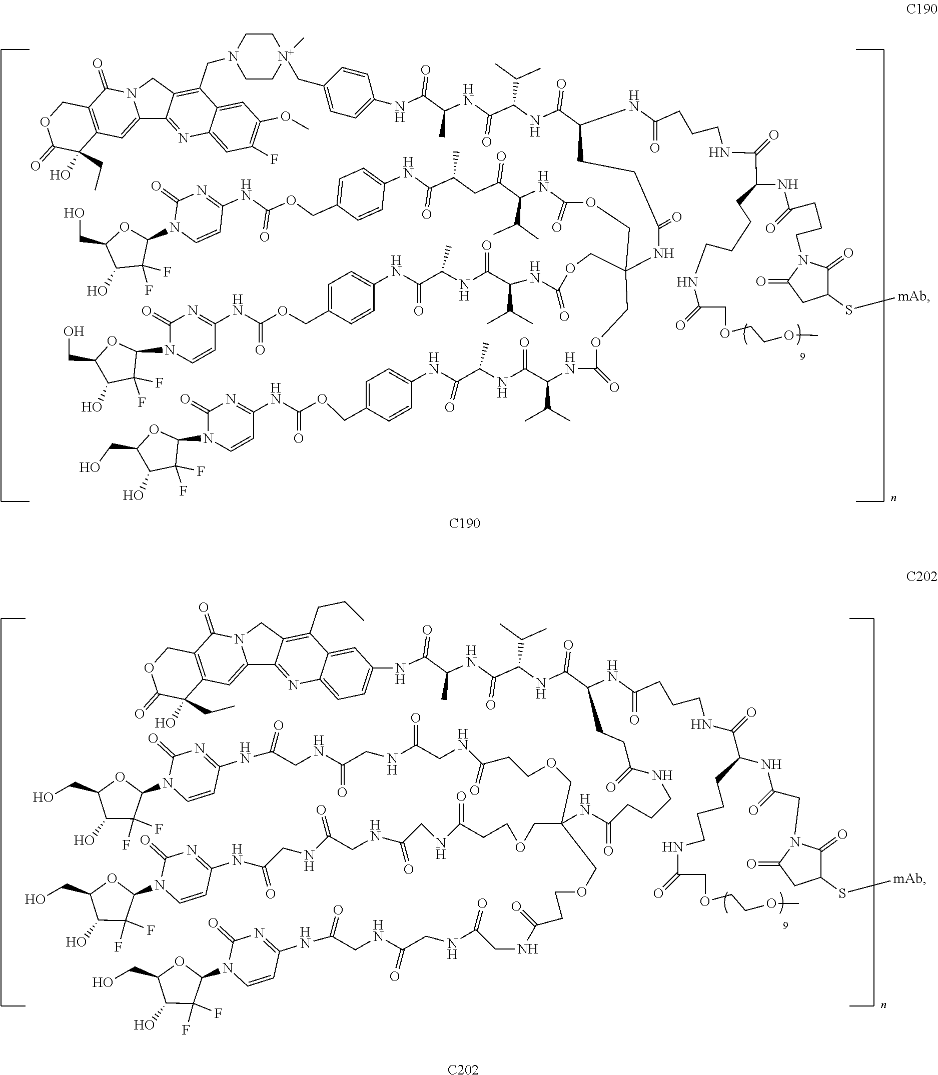 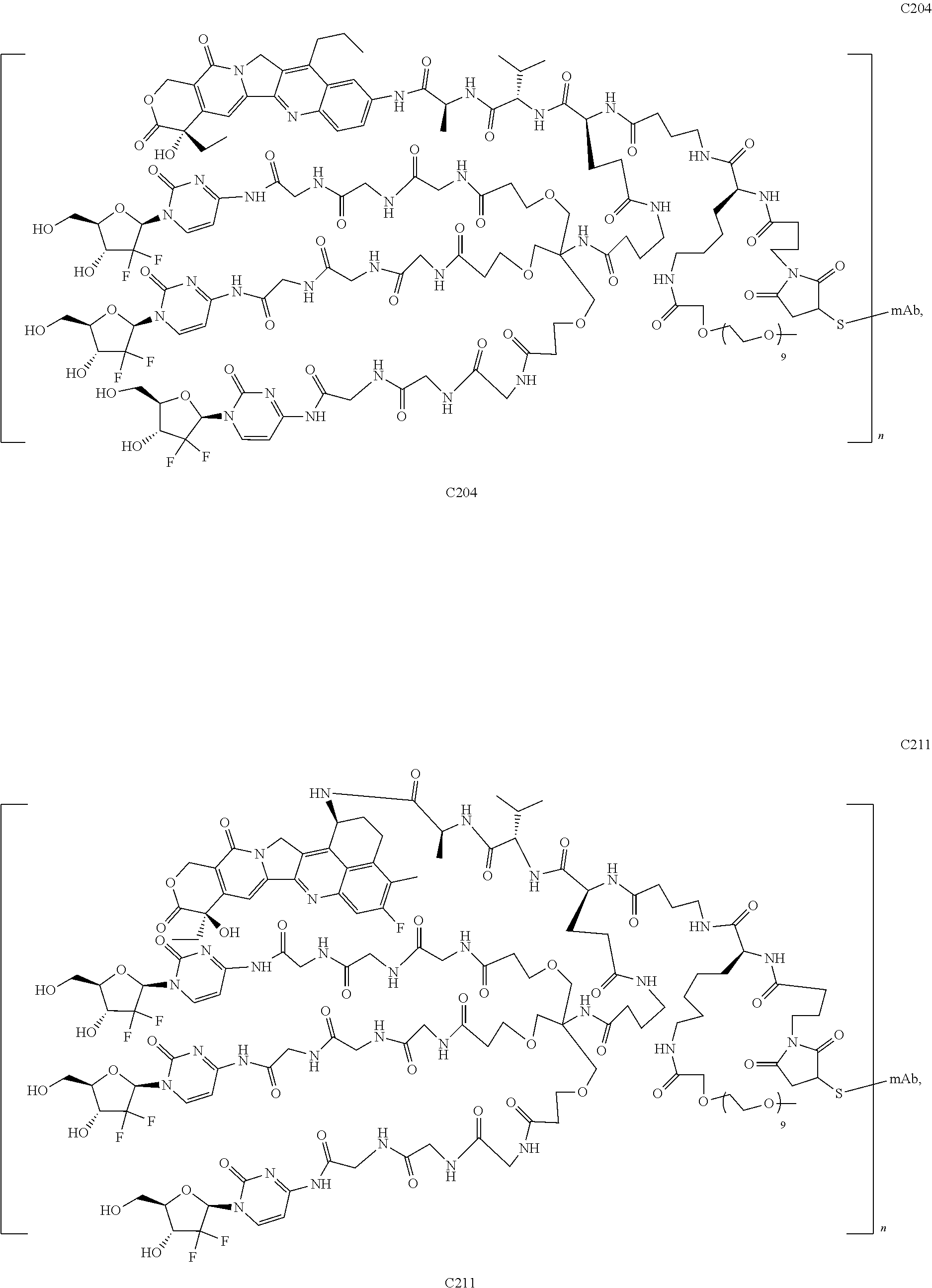 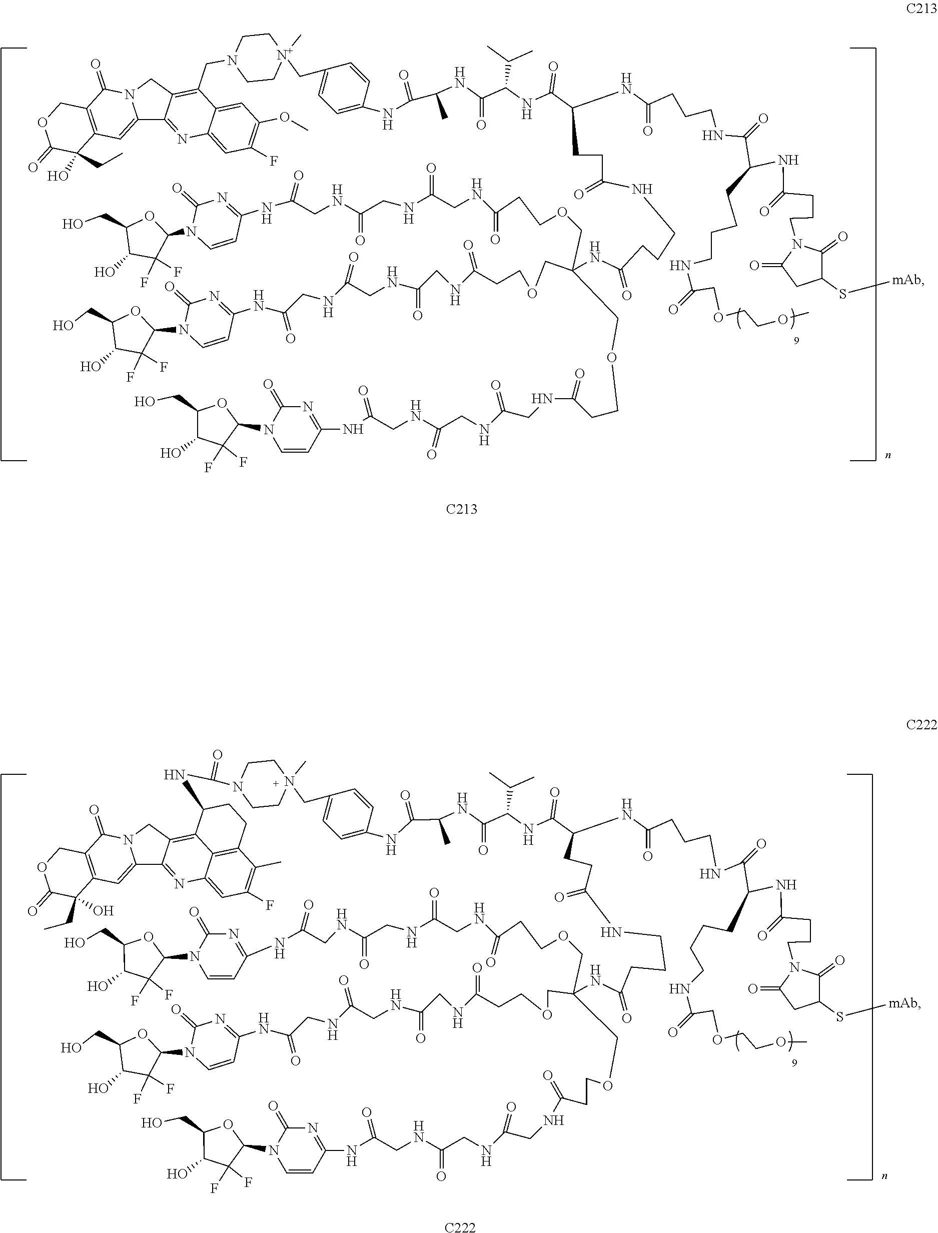 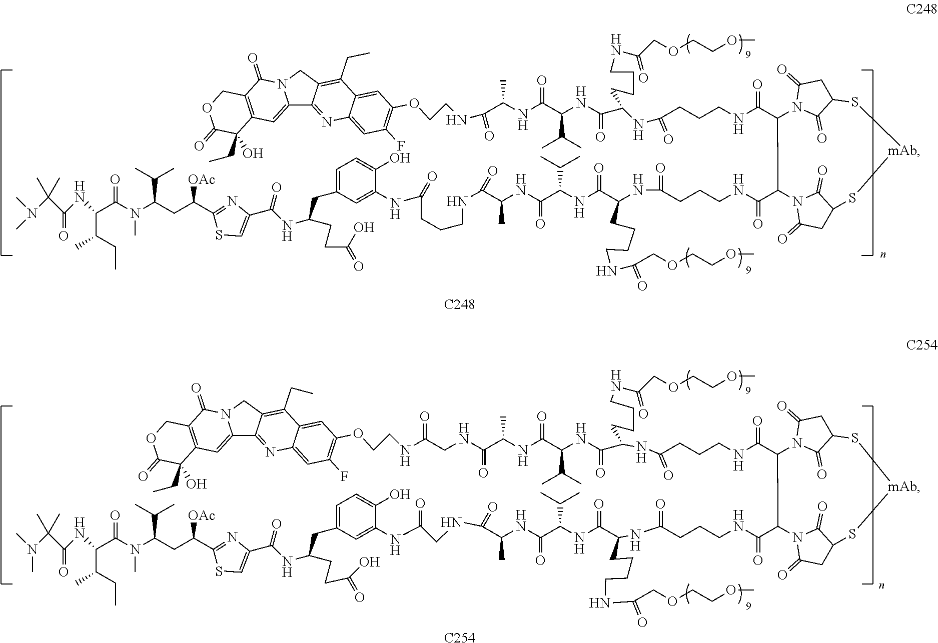 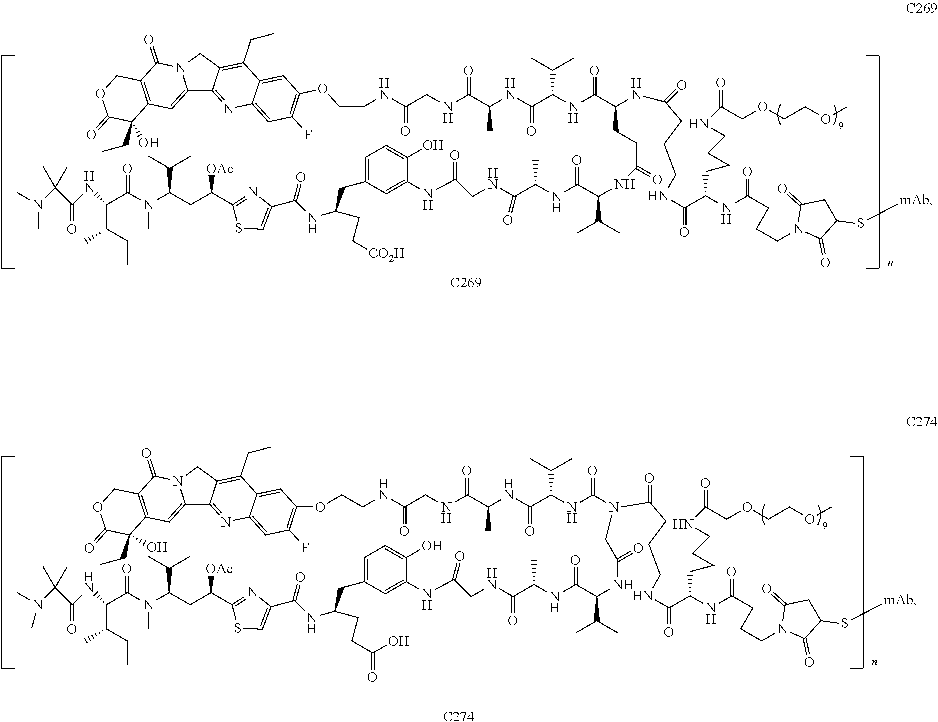 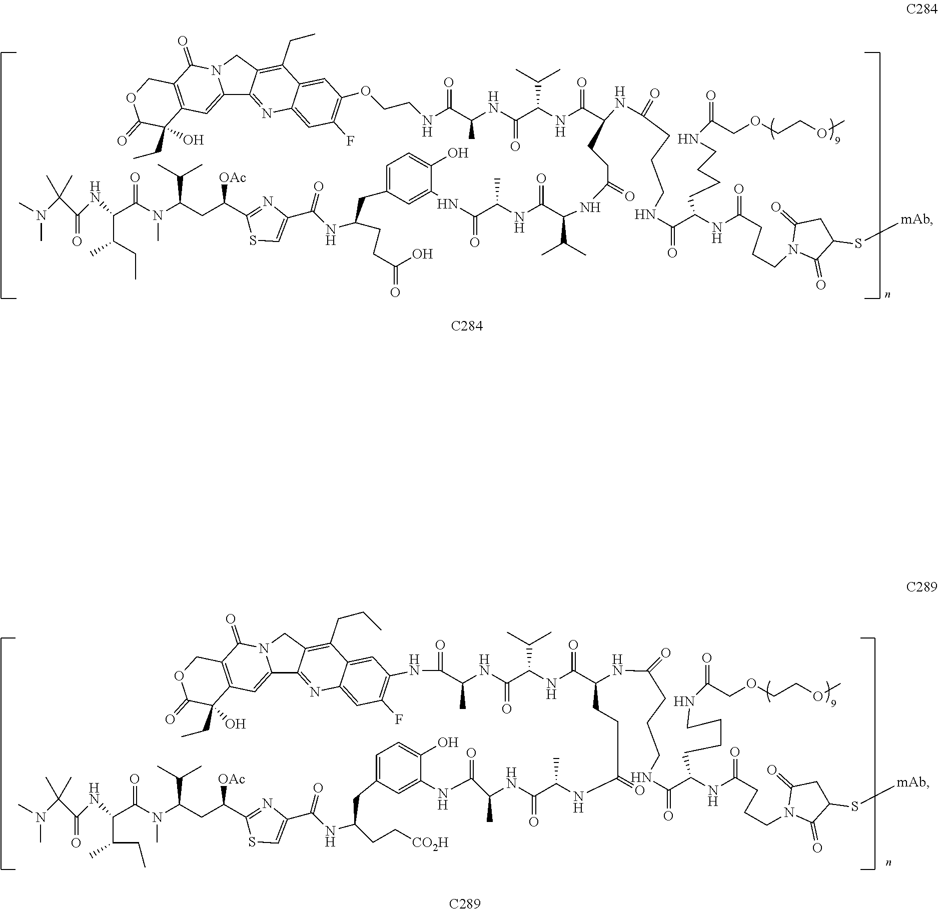 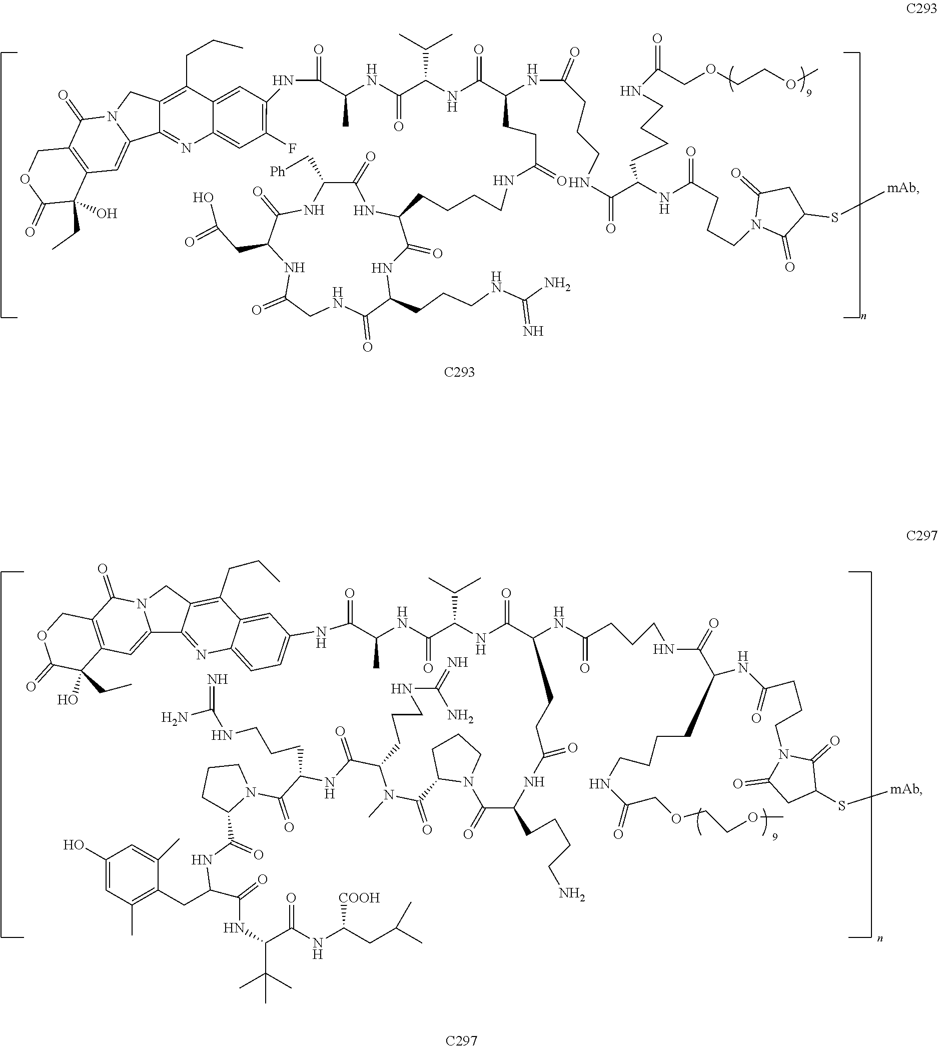 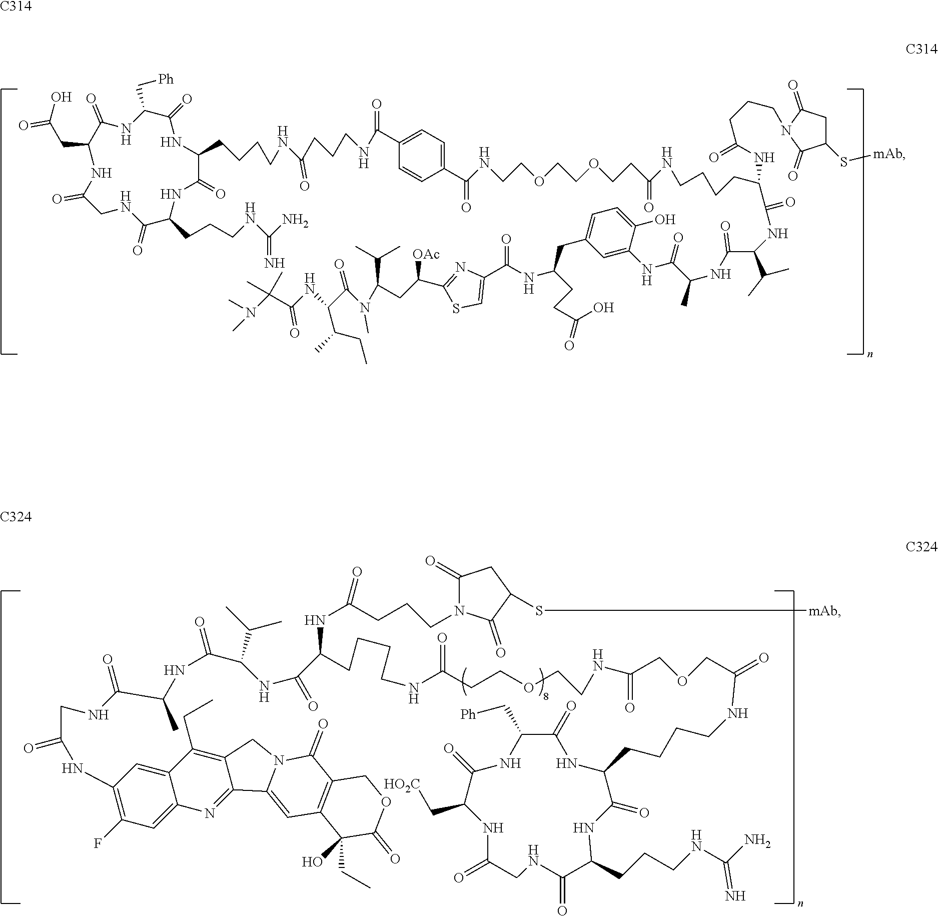 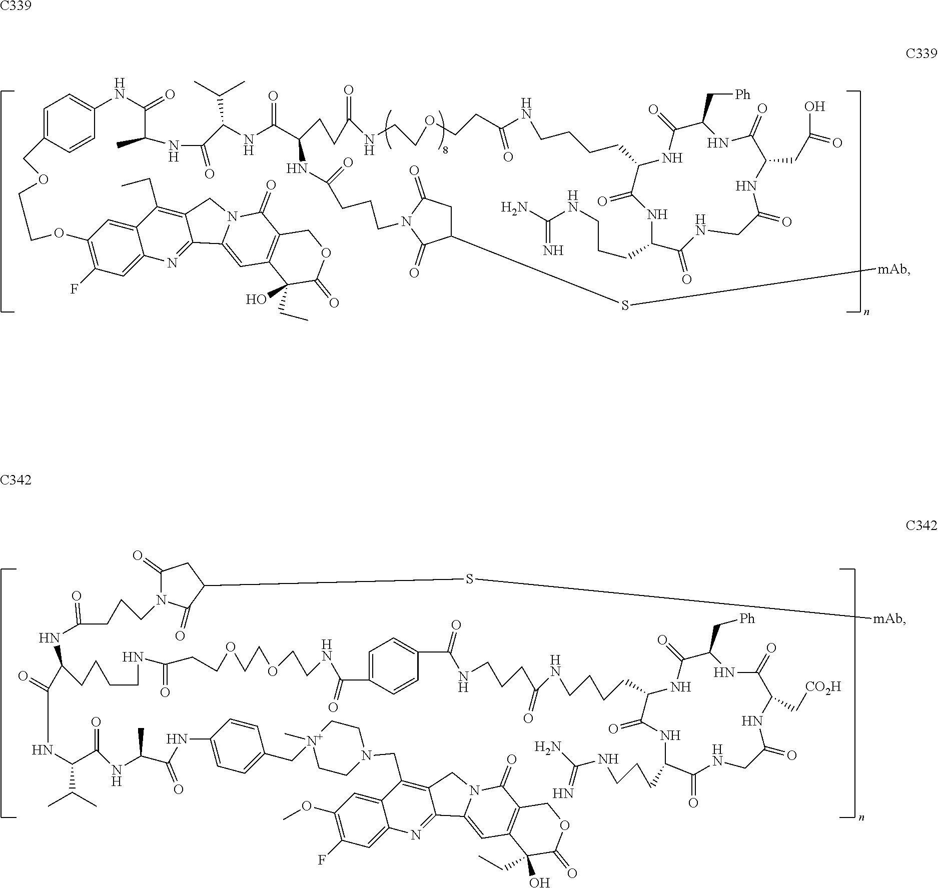 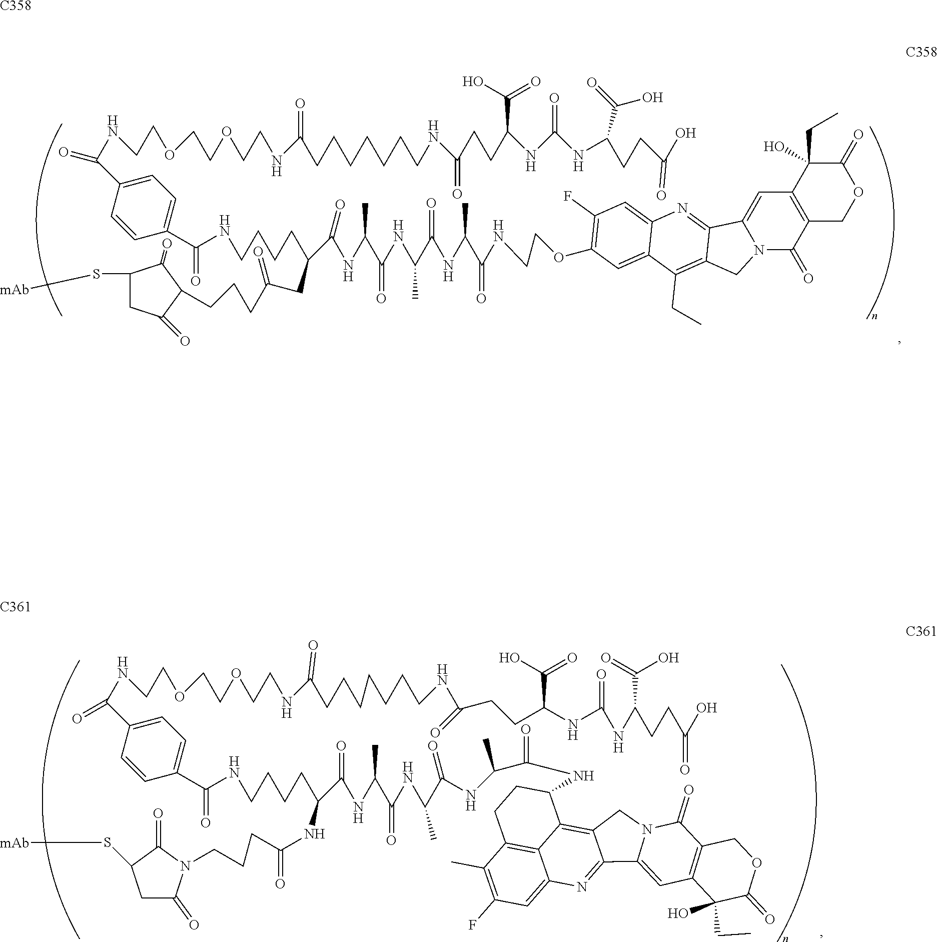 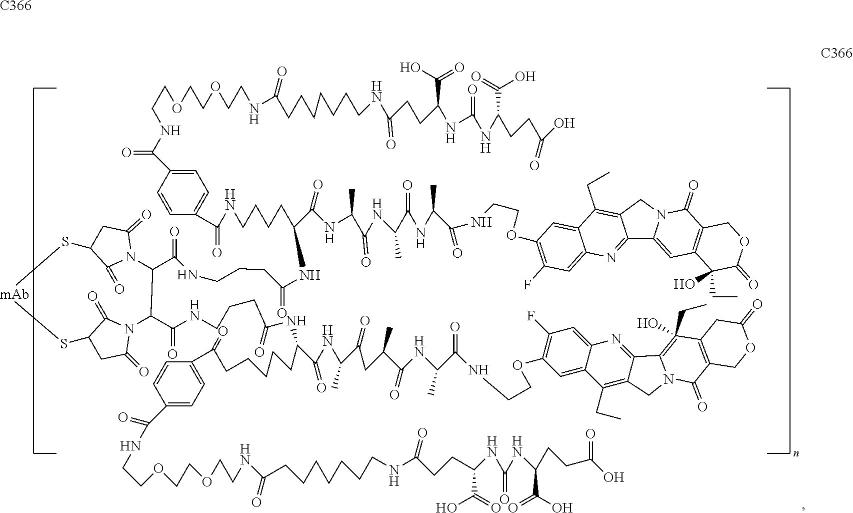 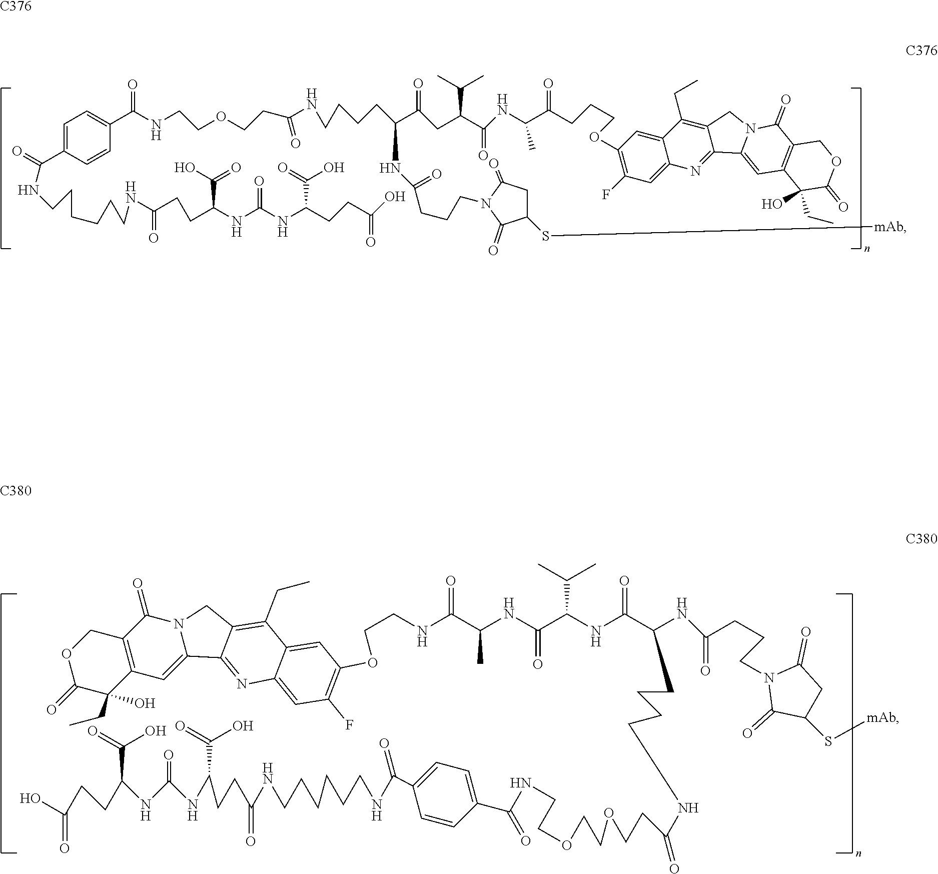 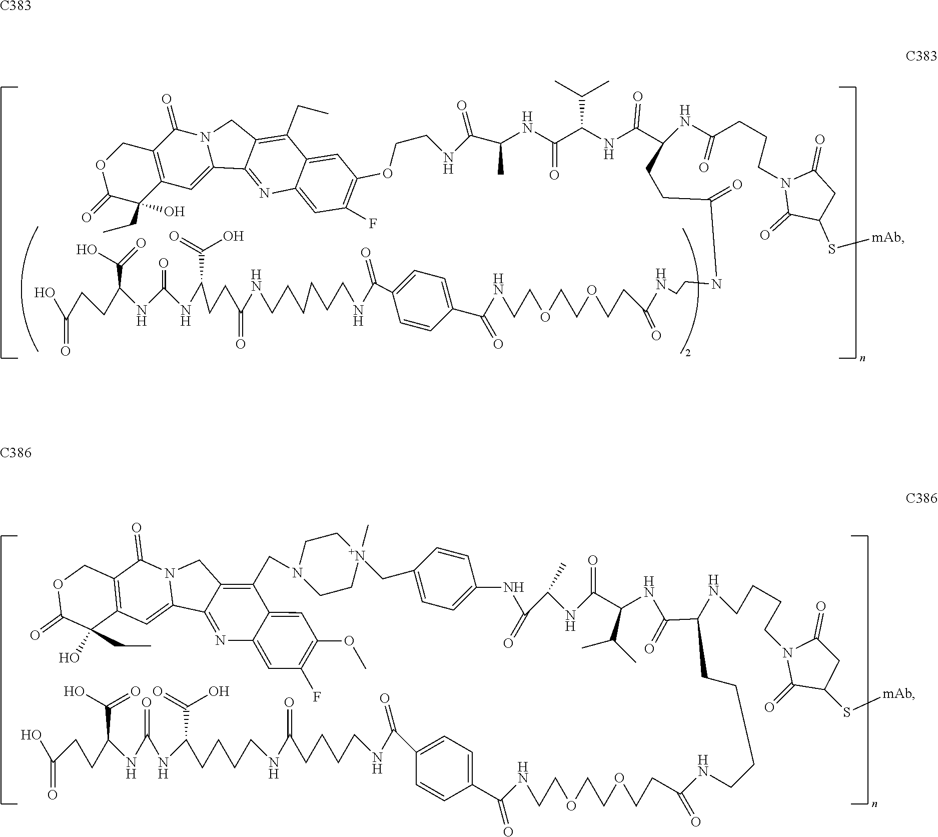 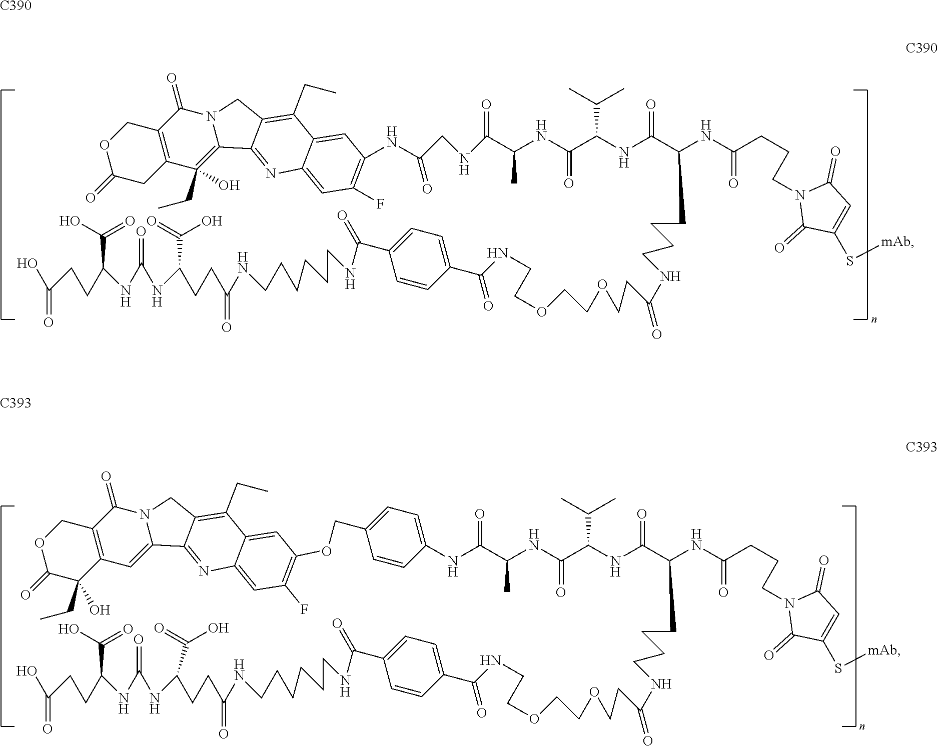 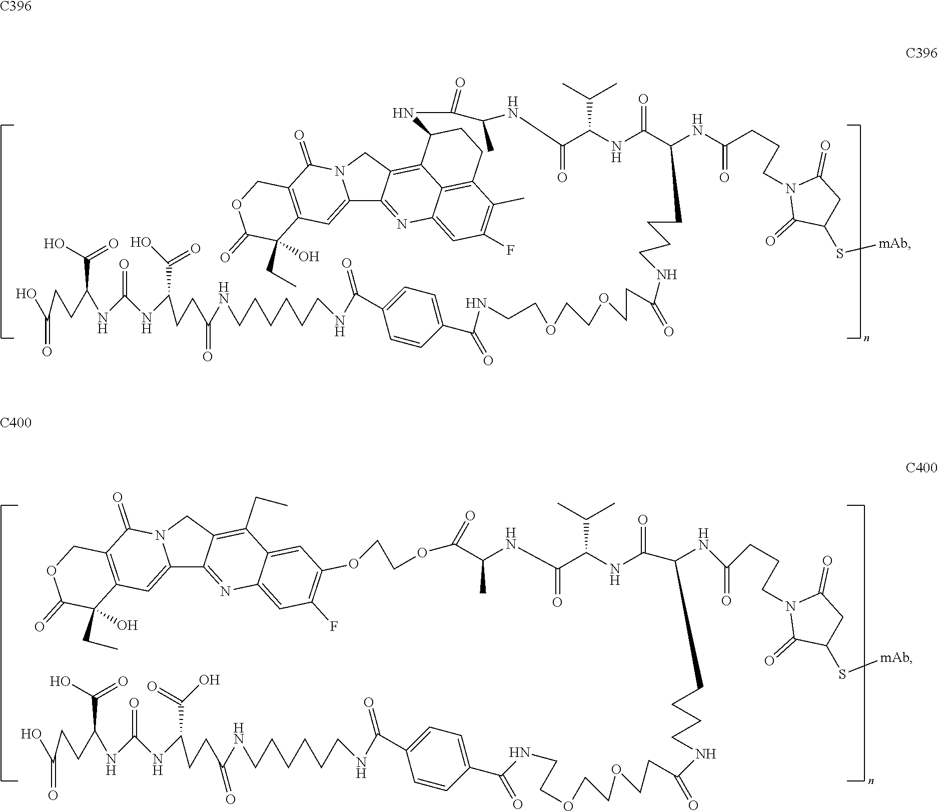 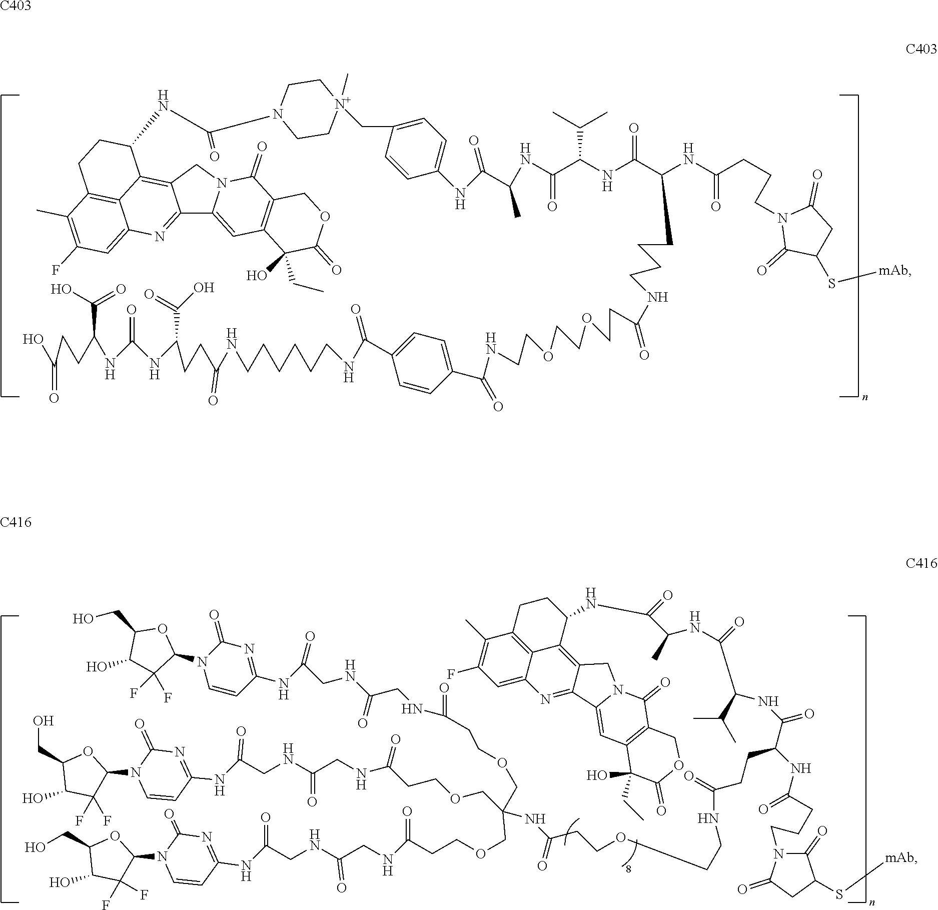 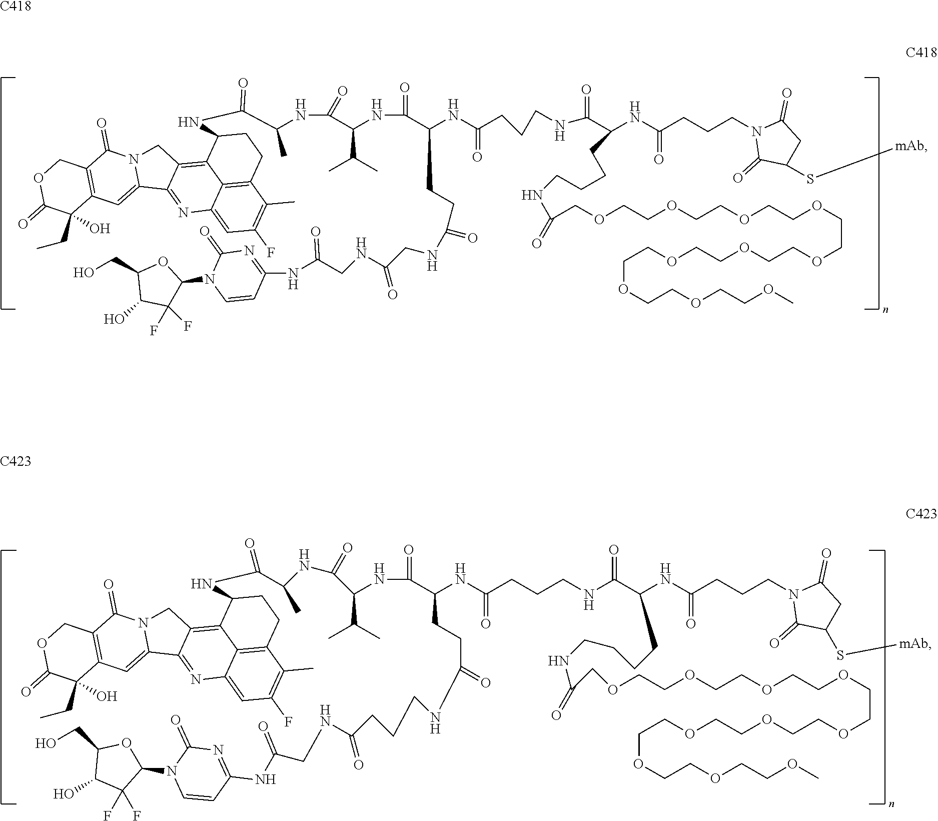 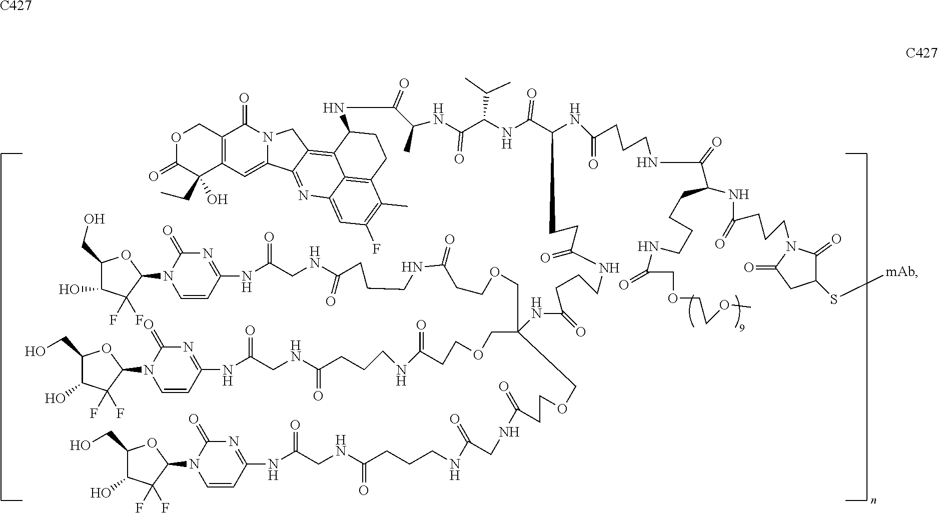 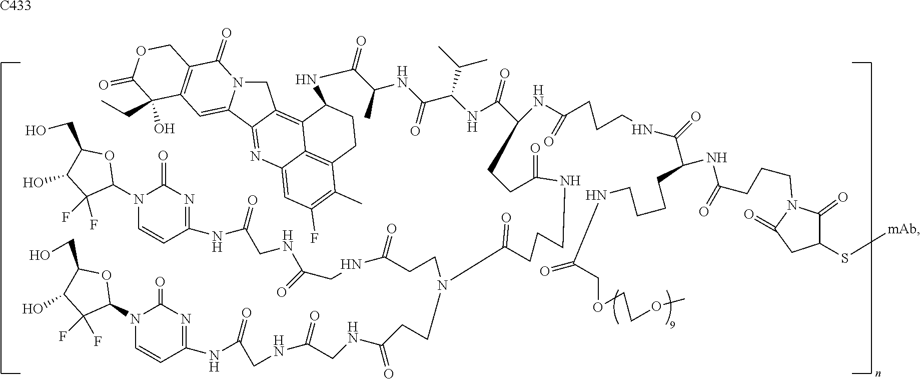 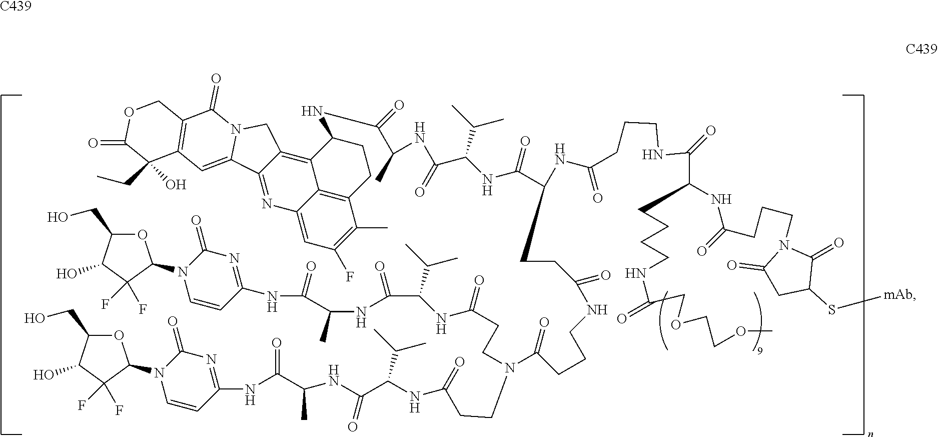 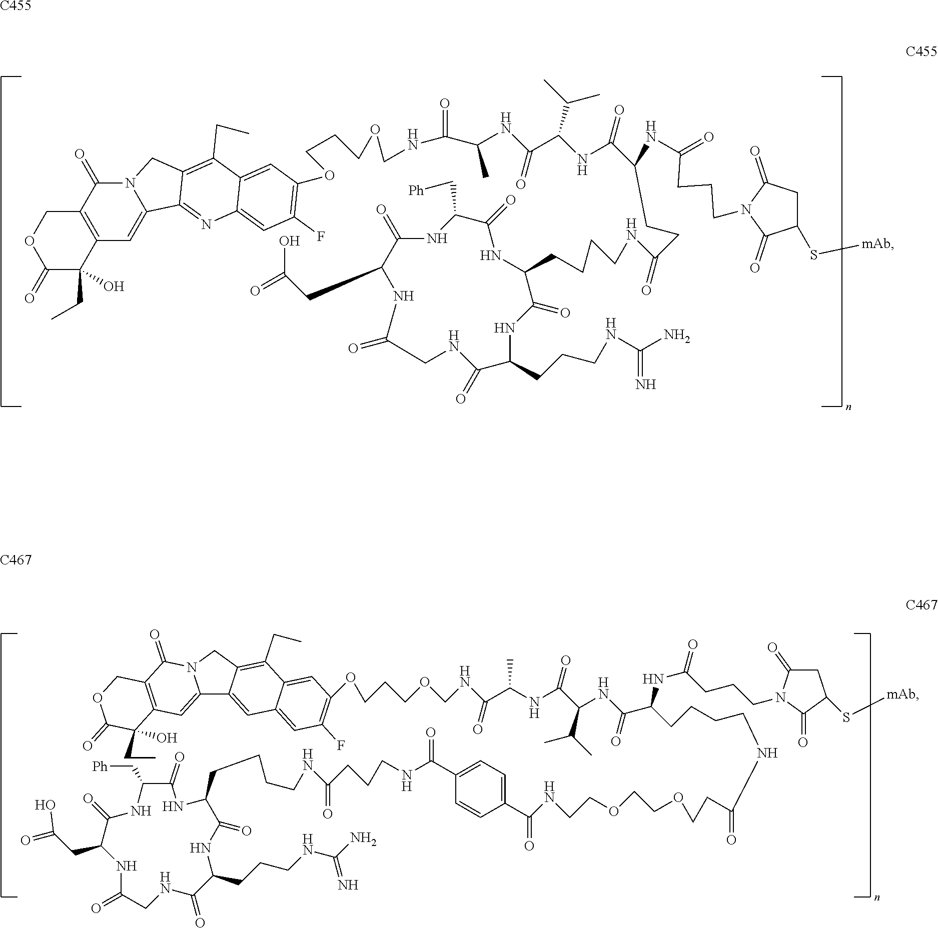 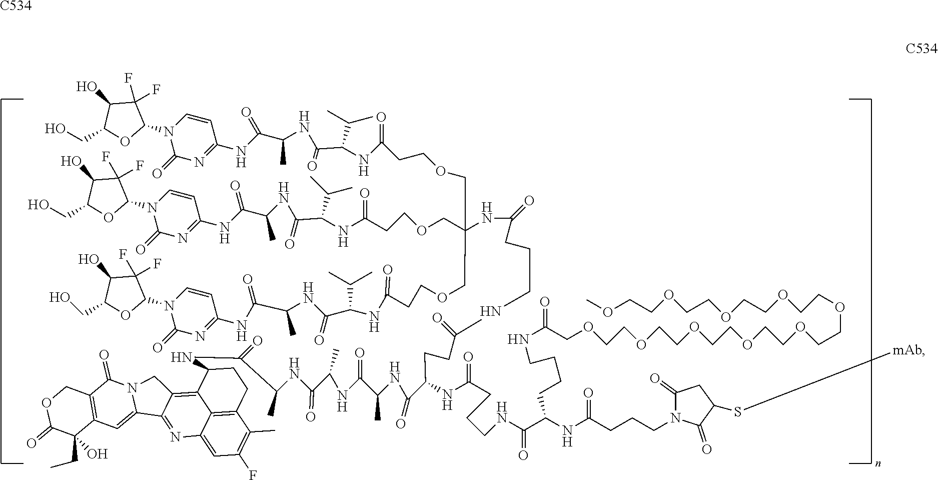 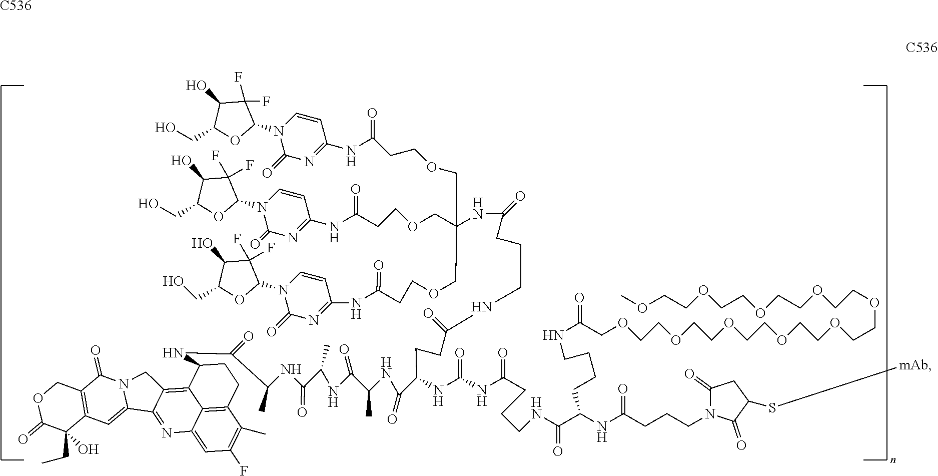 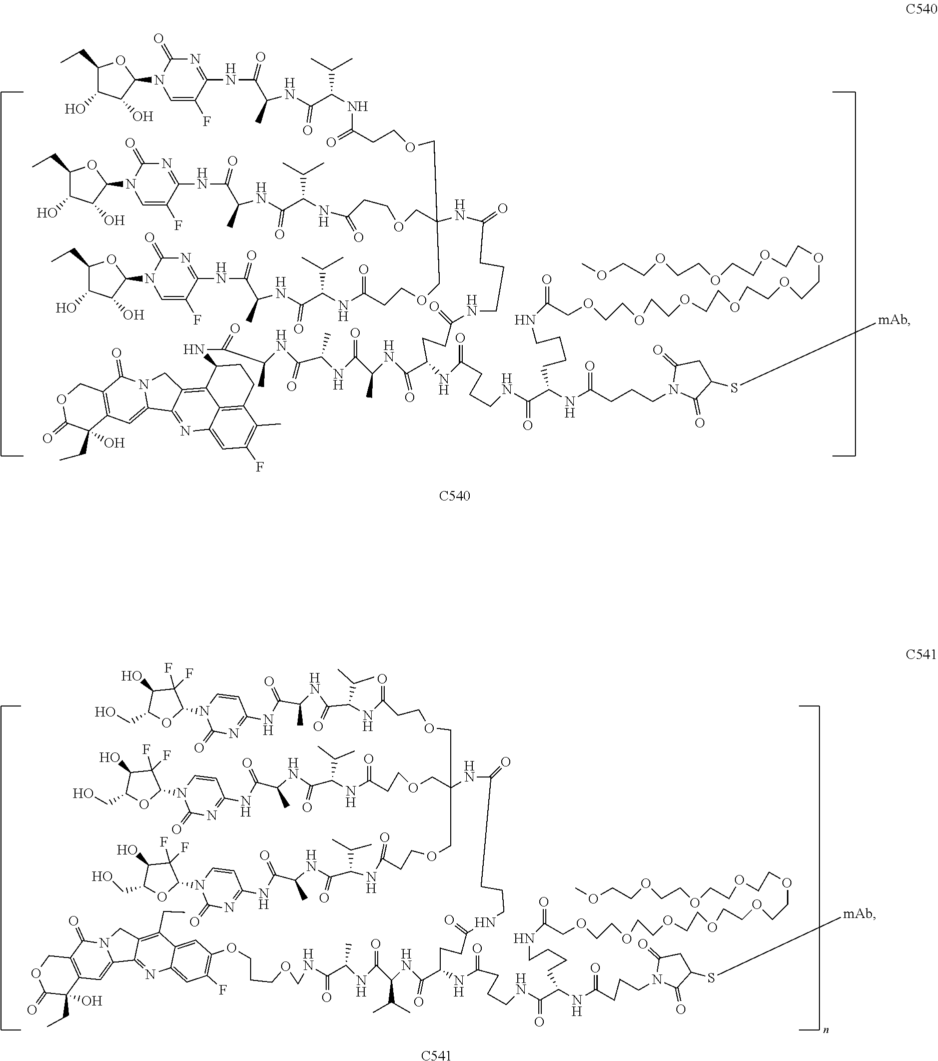 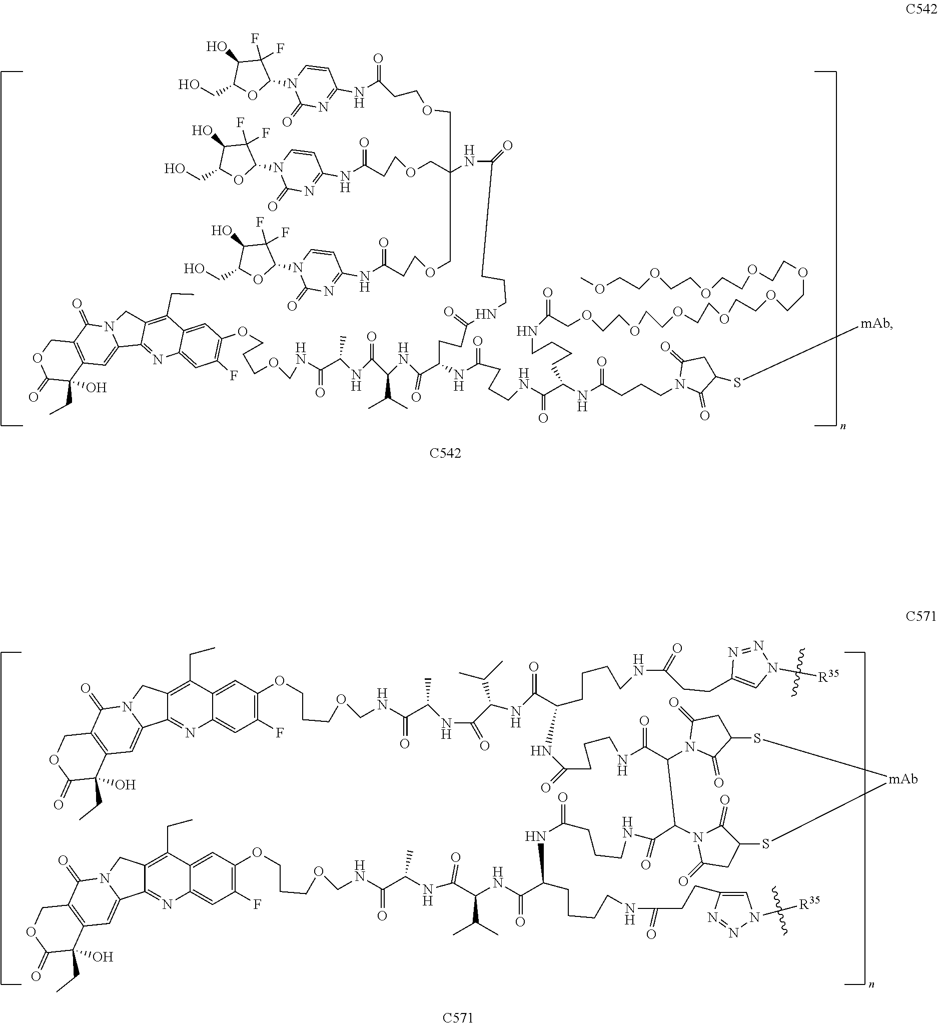 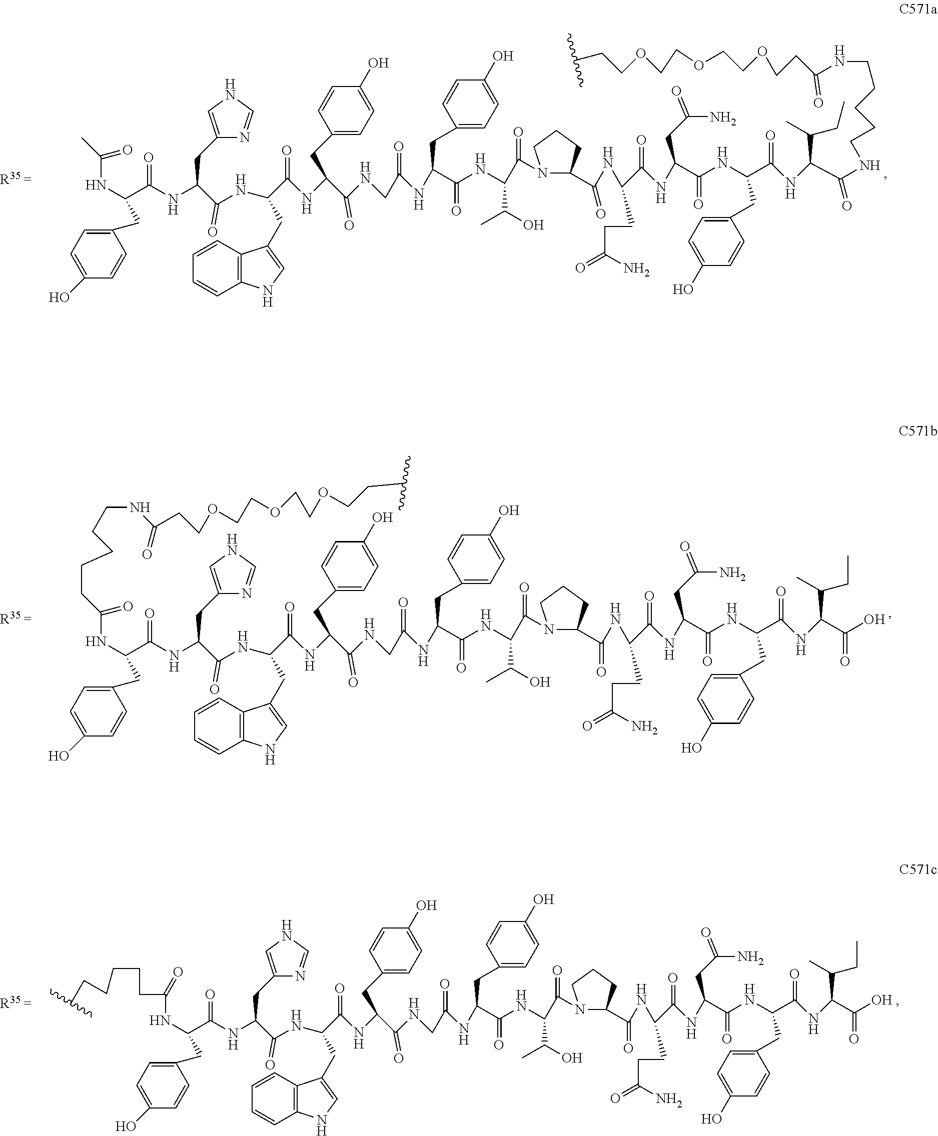 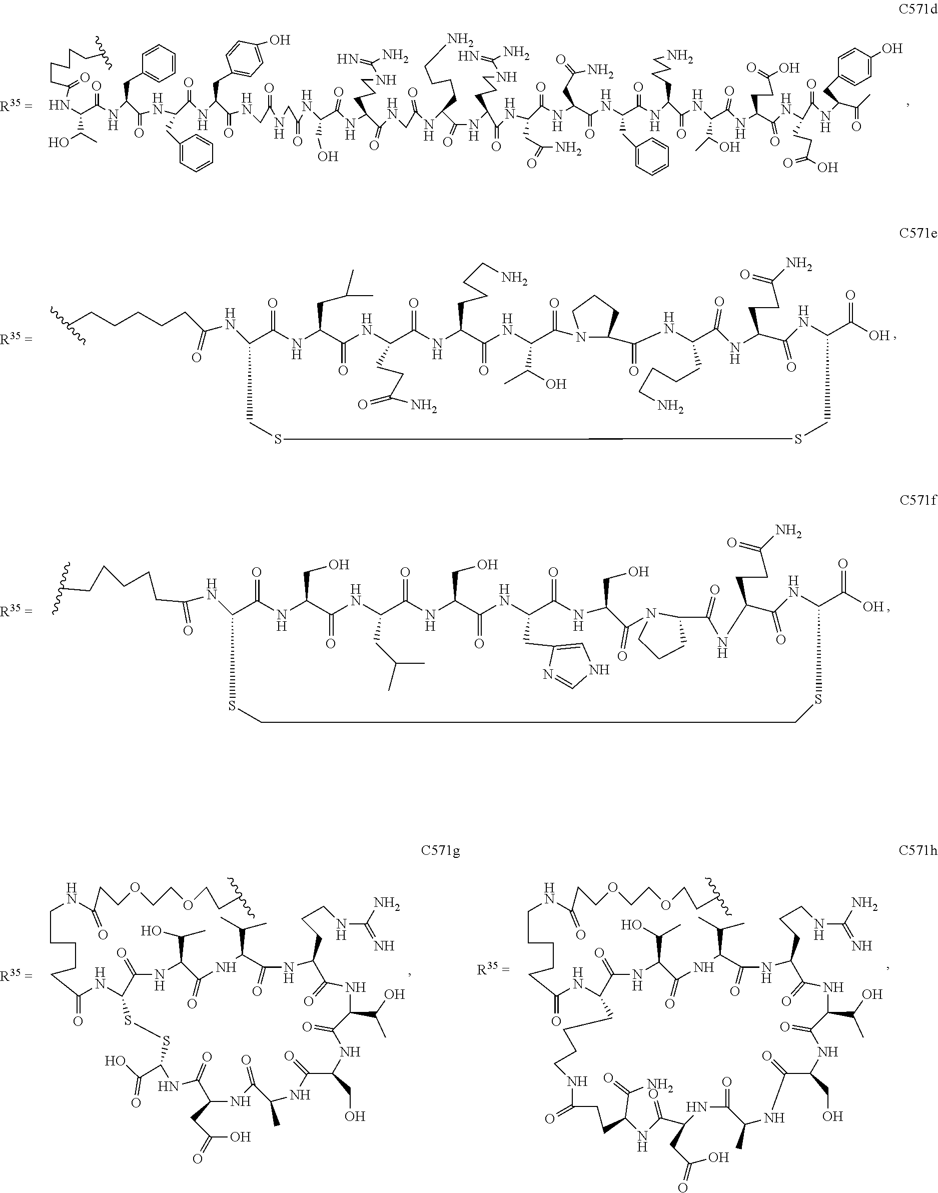 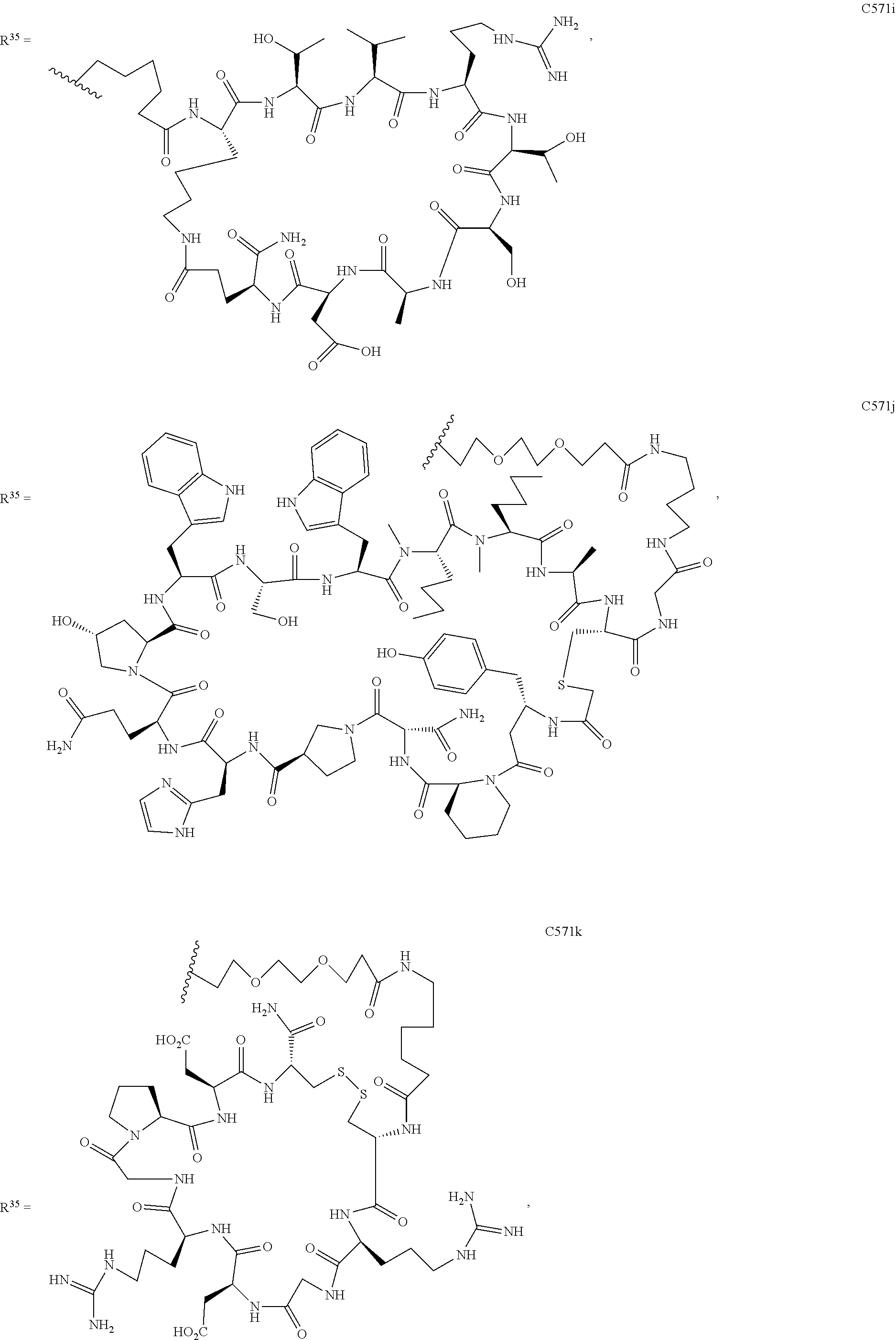 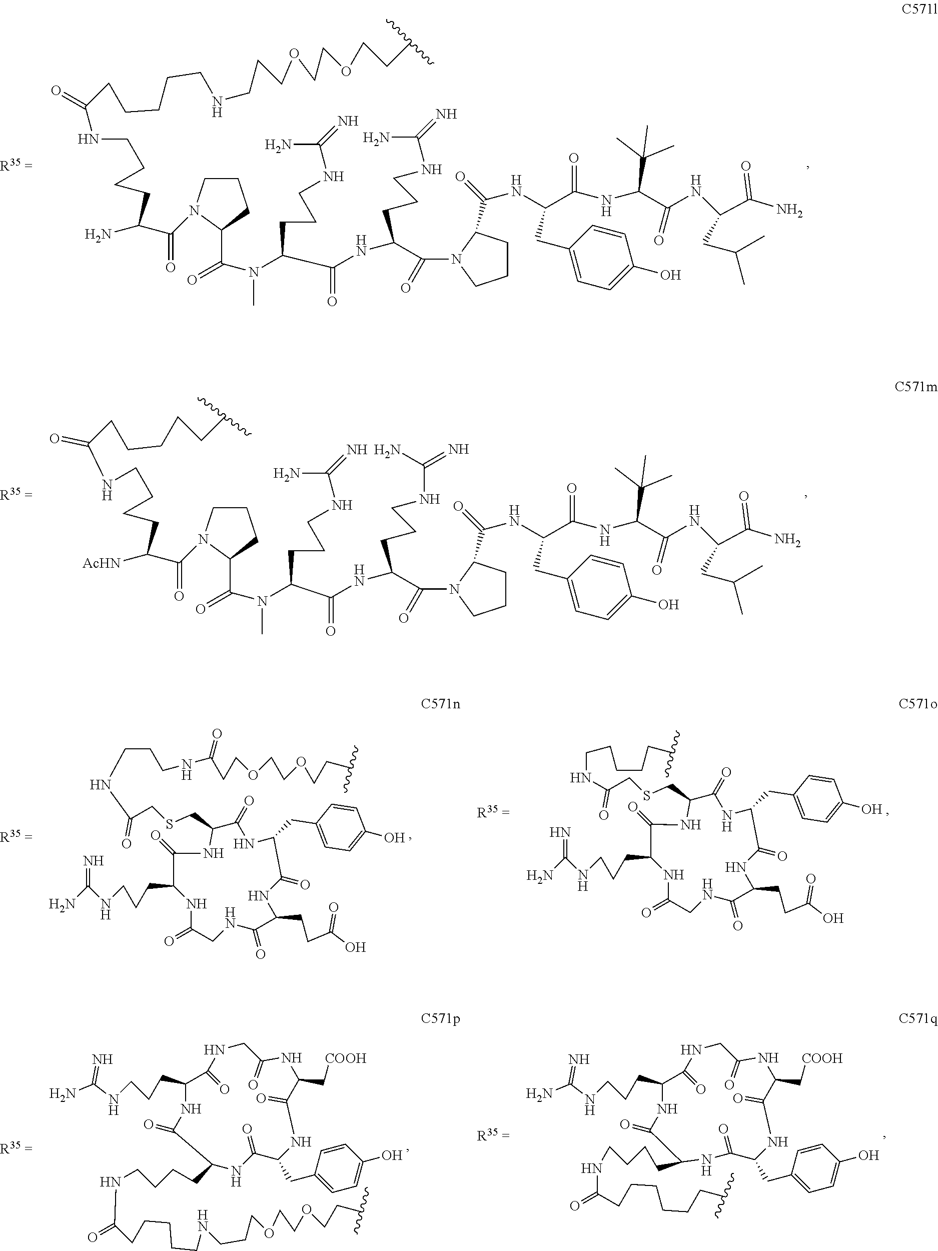 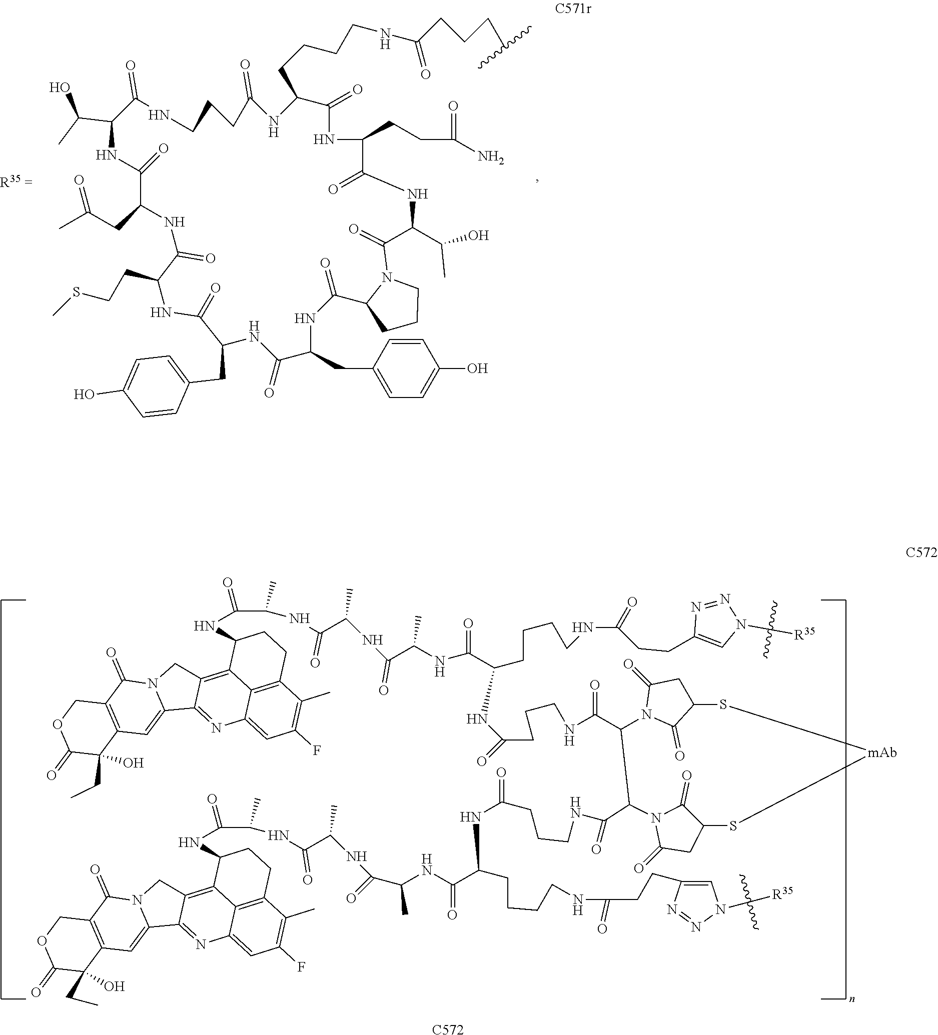 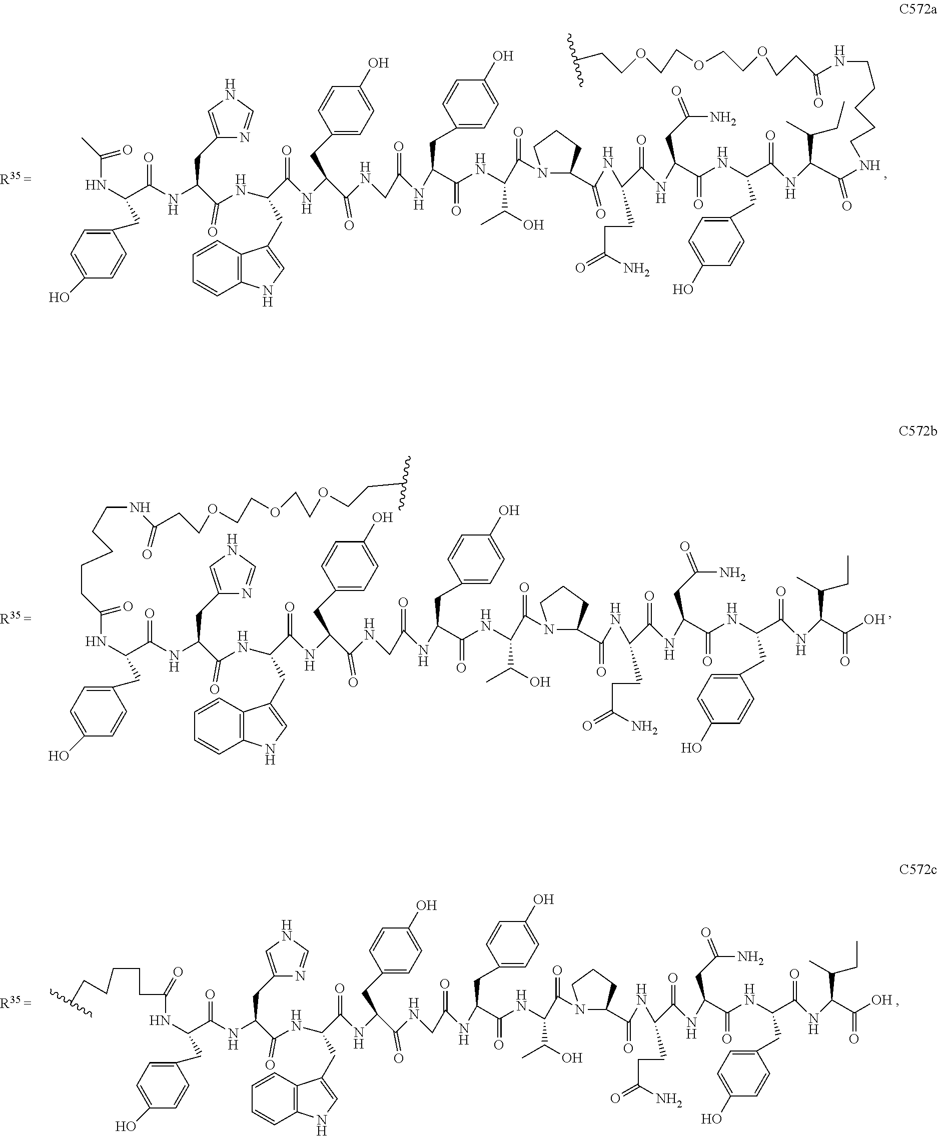 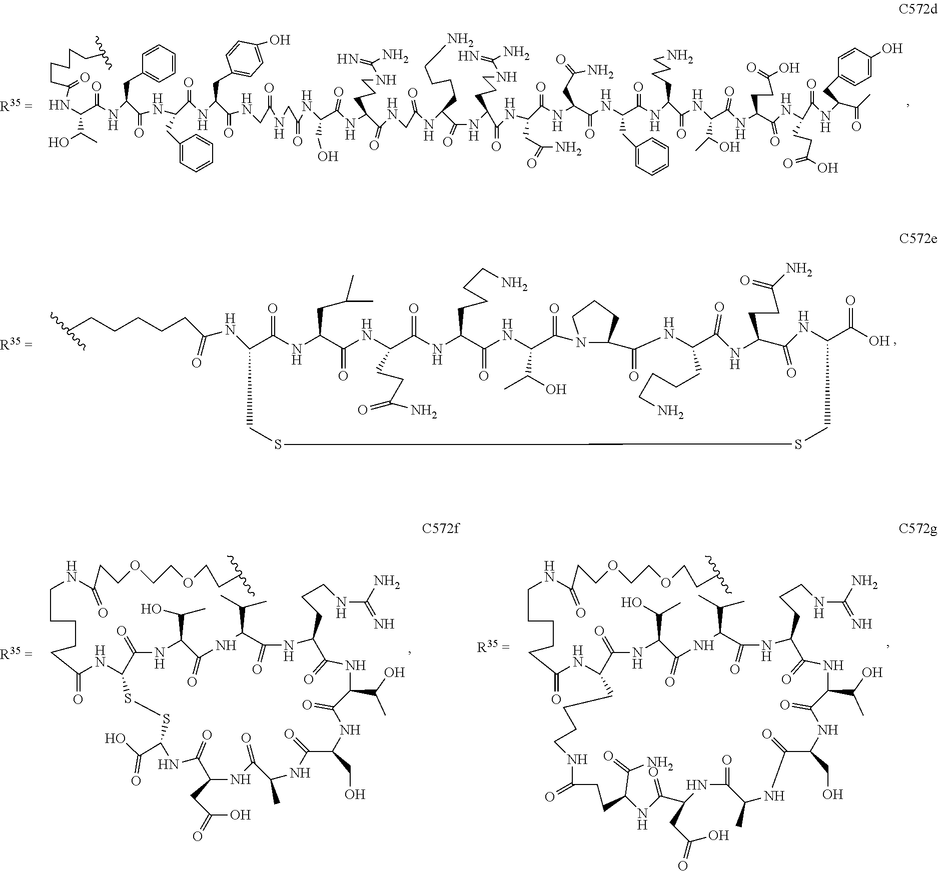 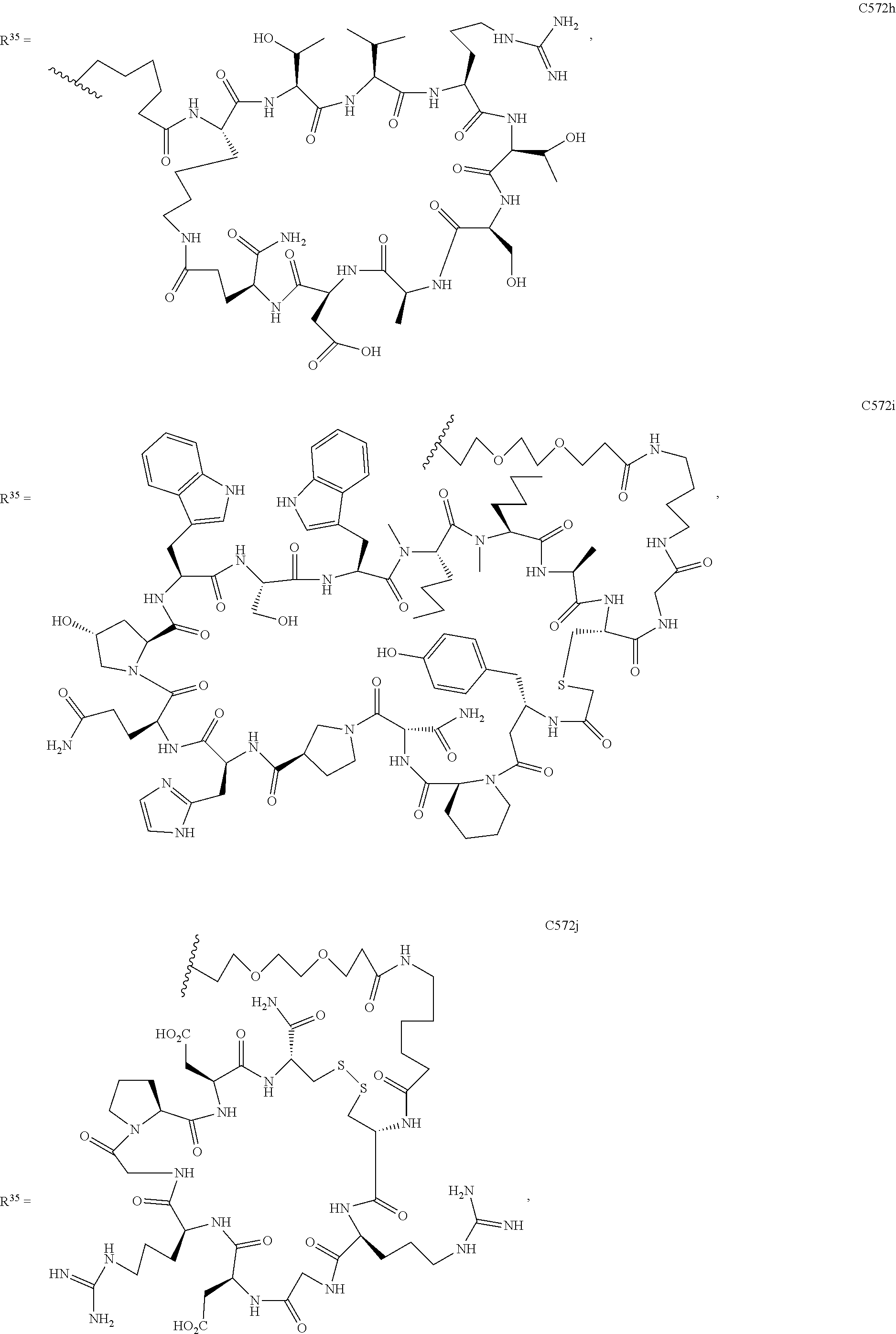 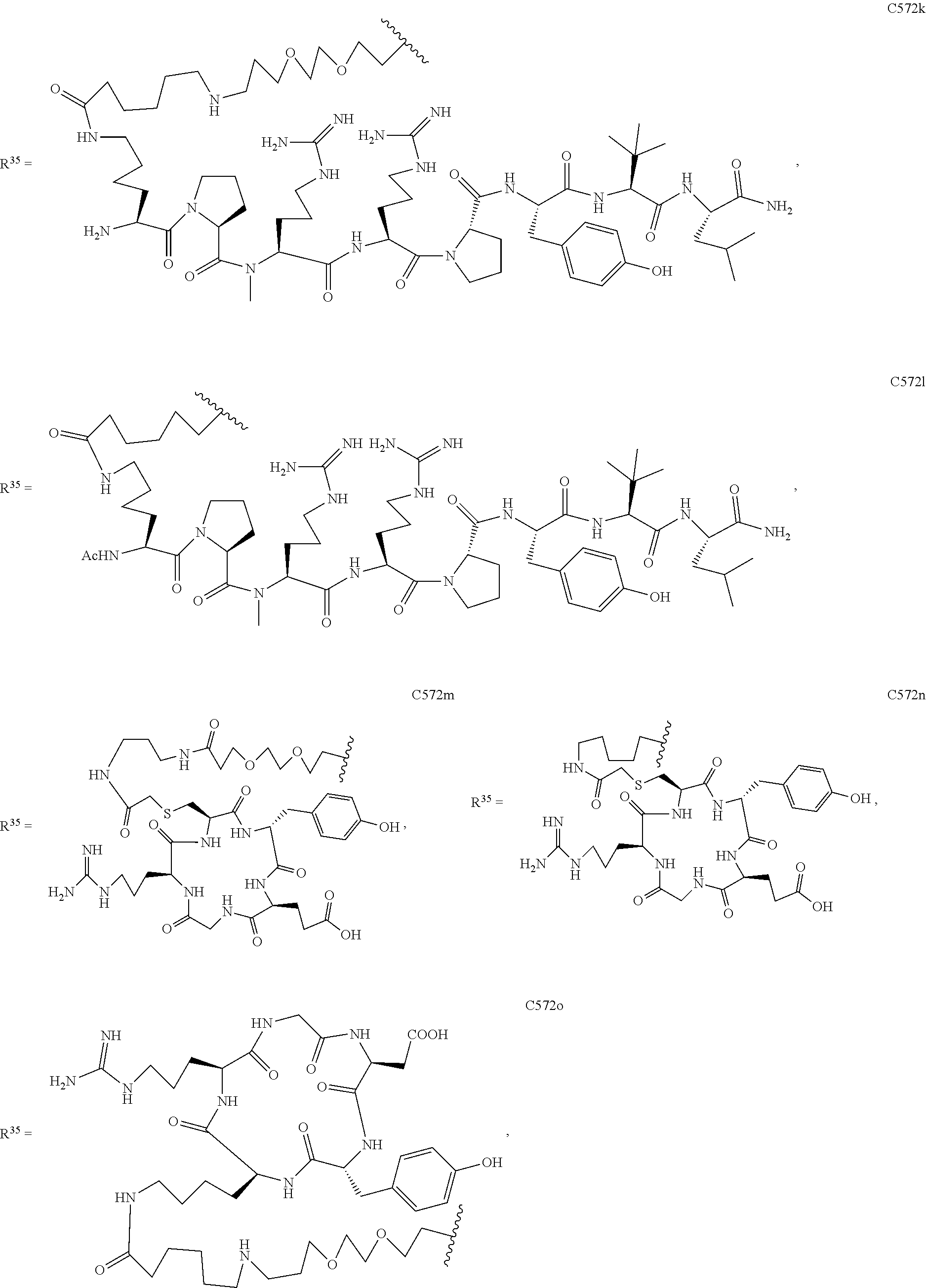 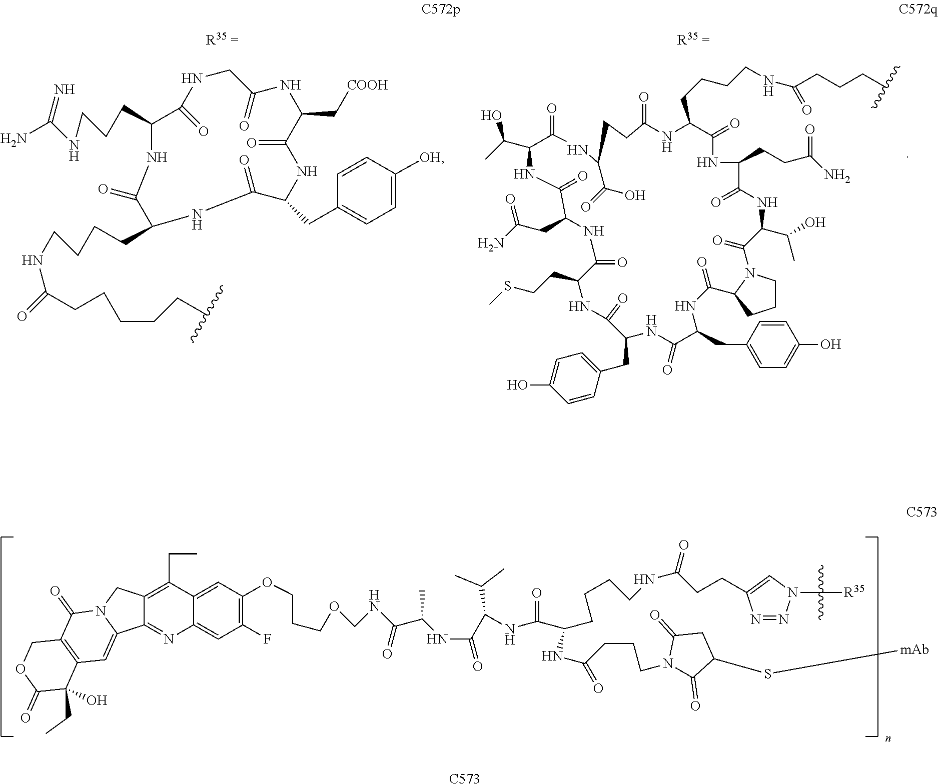 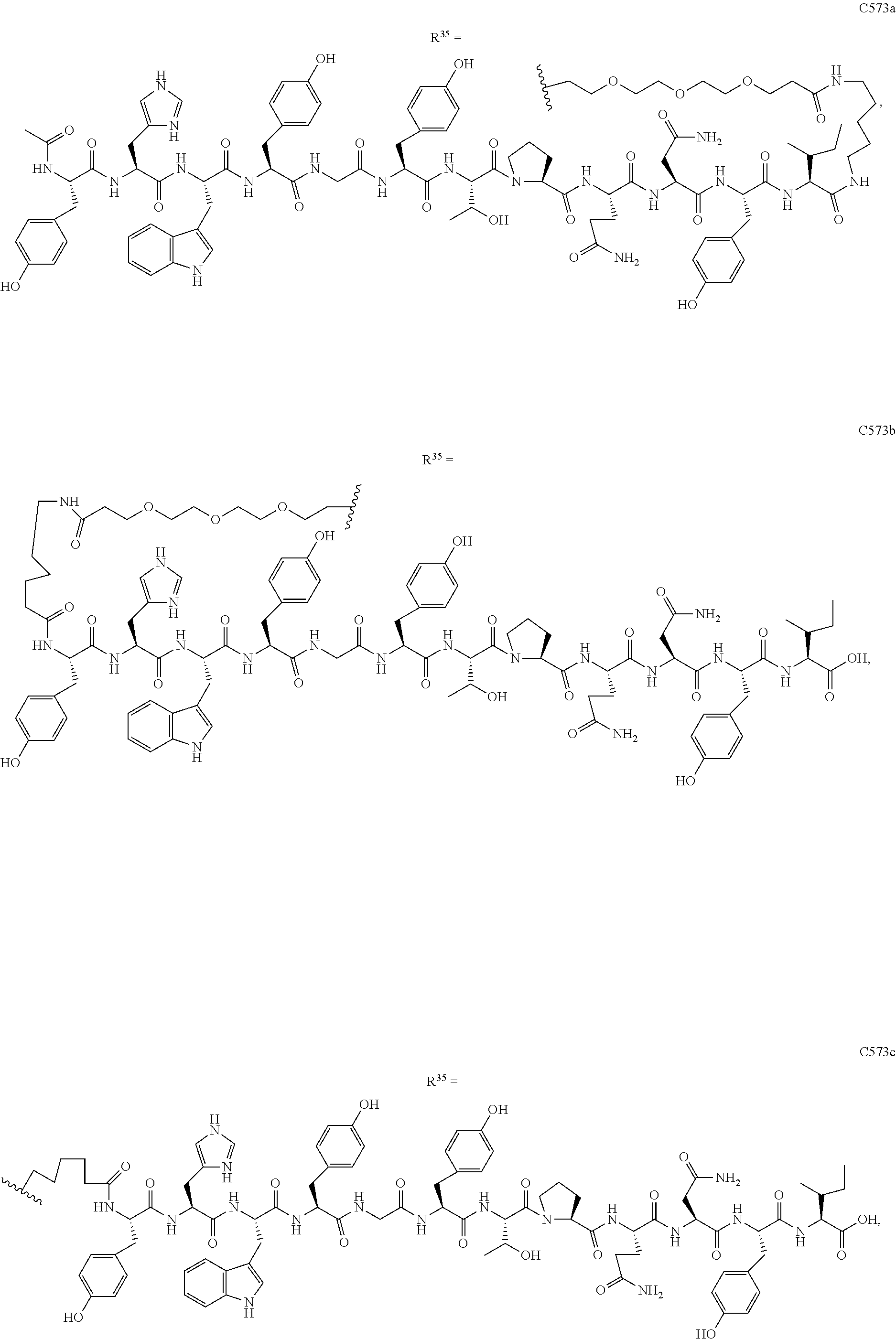 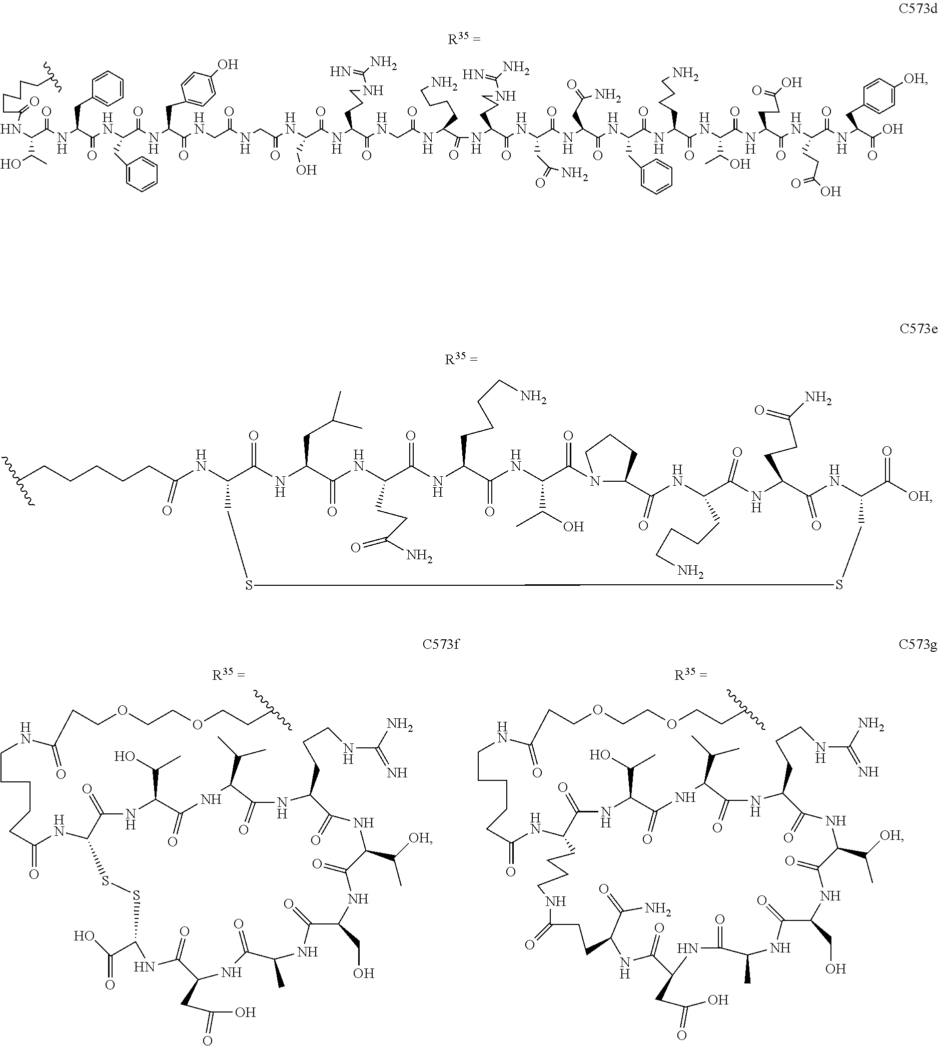 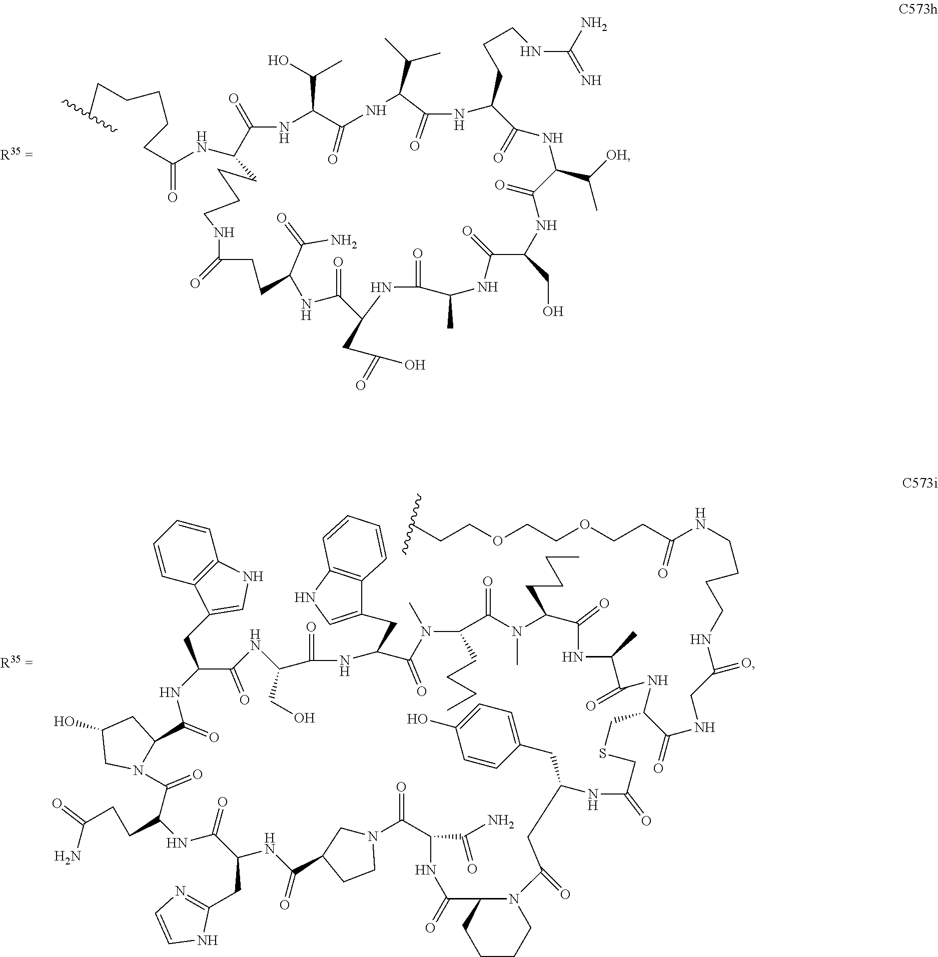 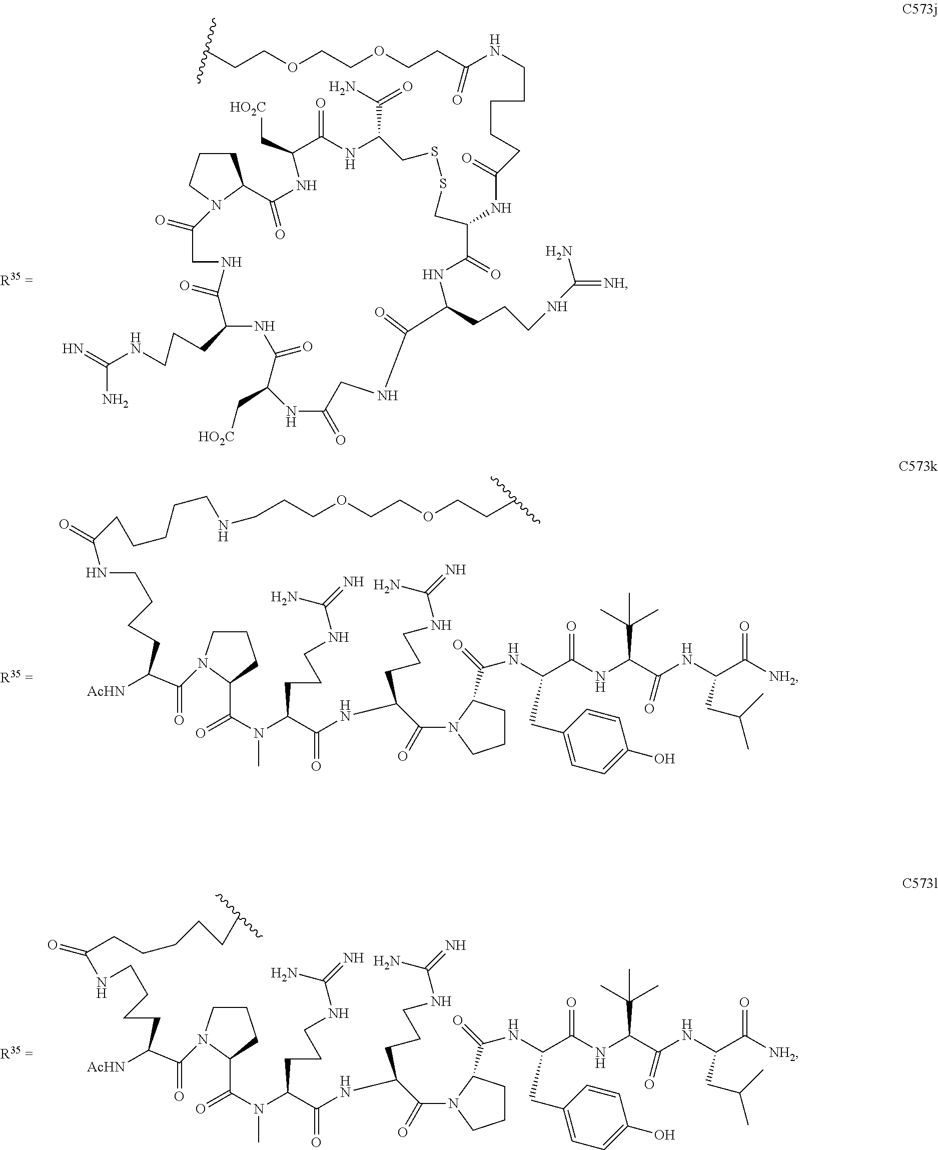 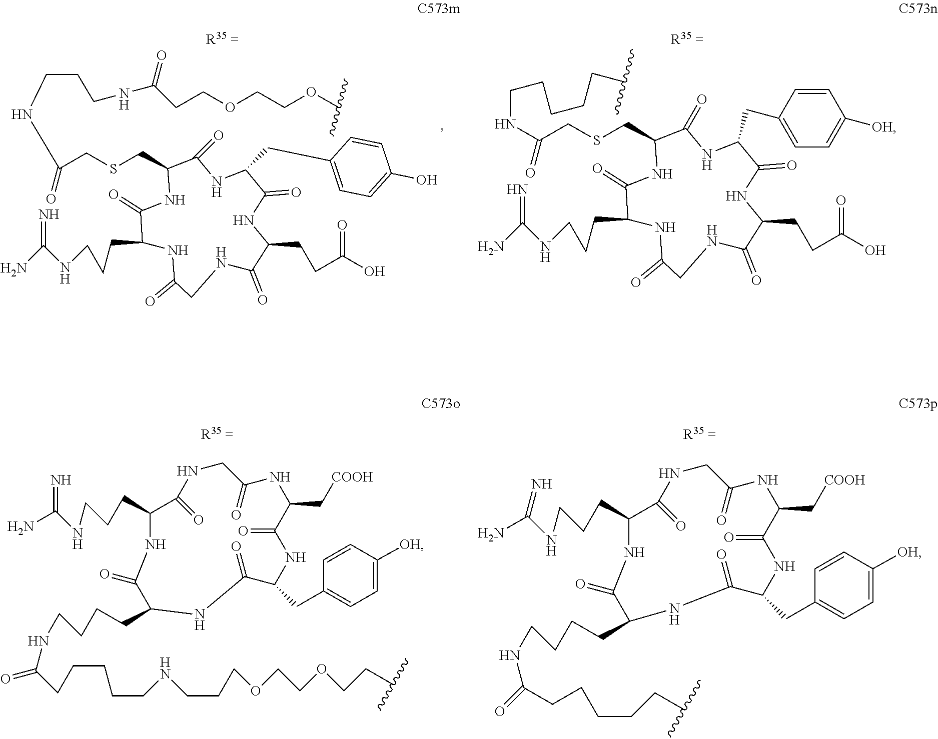 [SMILES: *OCCOCCC(=O)NCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O ; *CCCCCNC(=O)CSC[C@@H]1NC(=O)[C@H](CCCNC(=N)N)NC(=O)CNC(=O)[C@H](CCC(=O)O)NC(=O)[C@@H](Cc2ccc(O)cc2)NC1=O ; *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O] 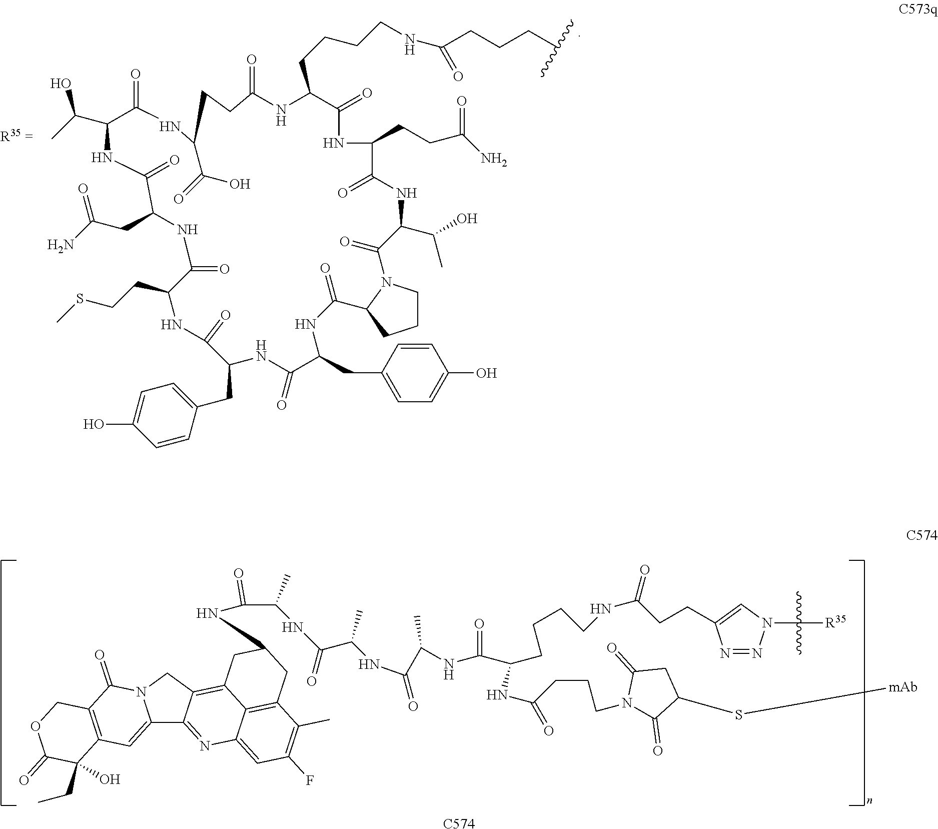 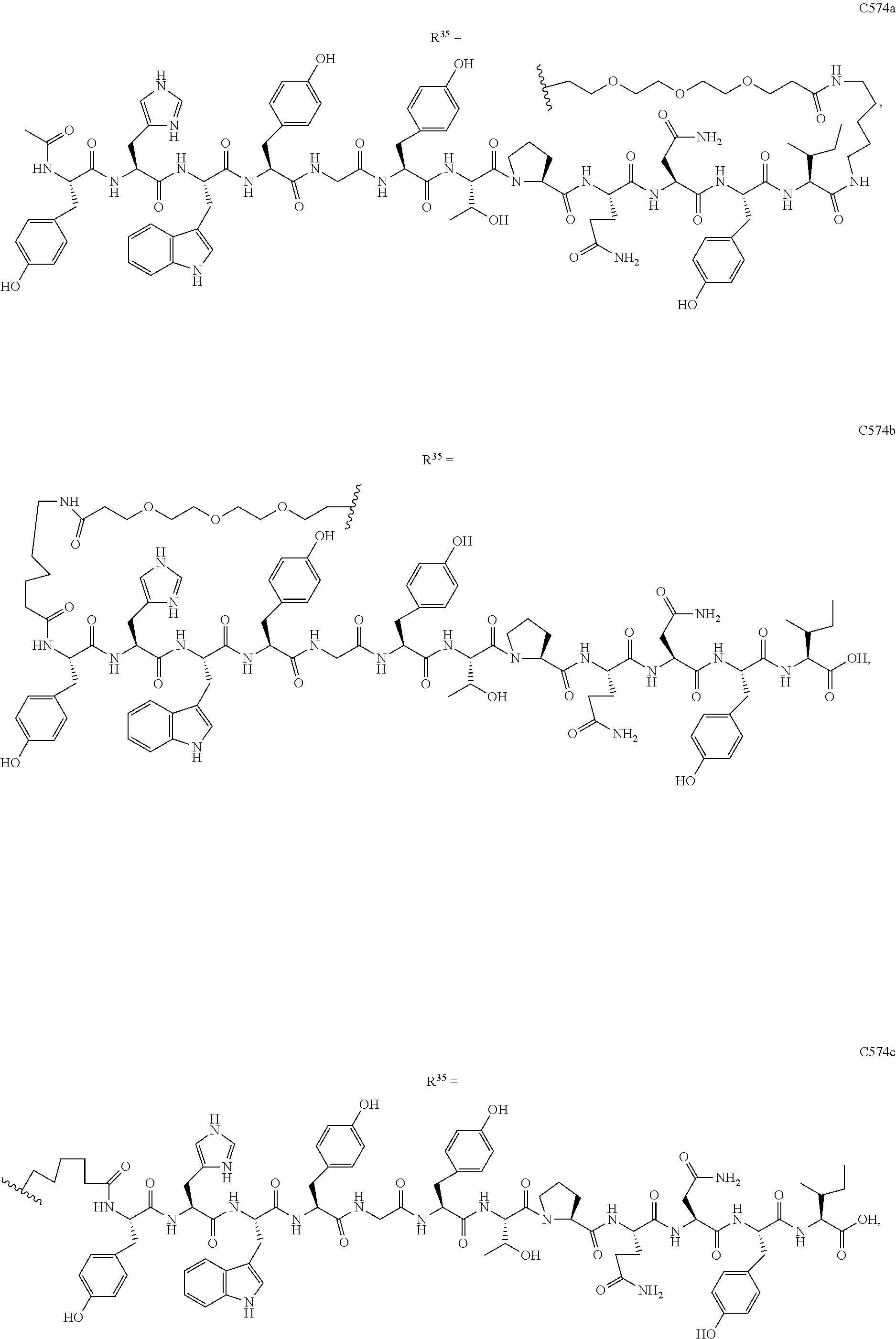 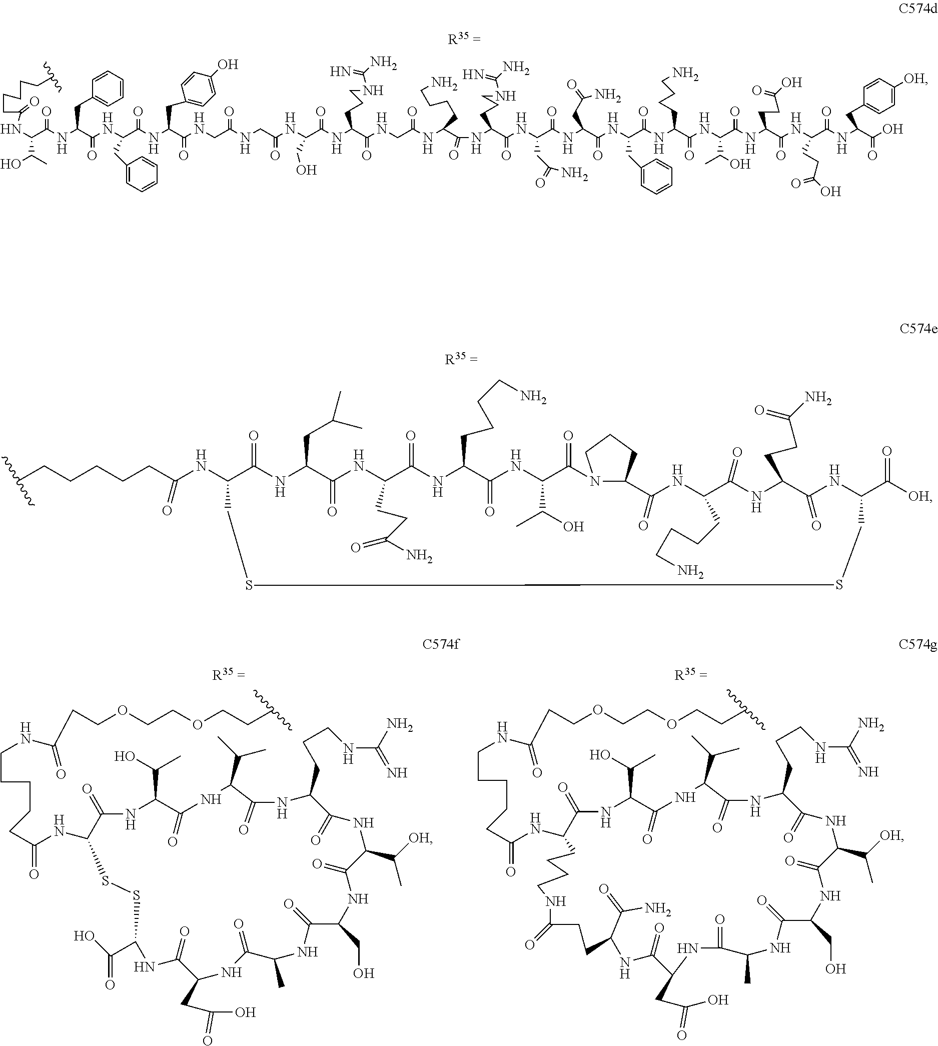 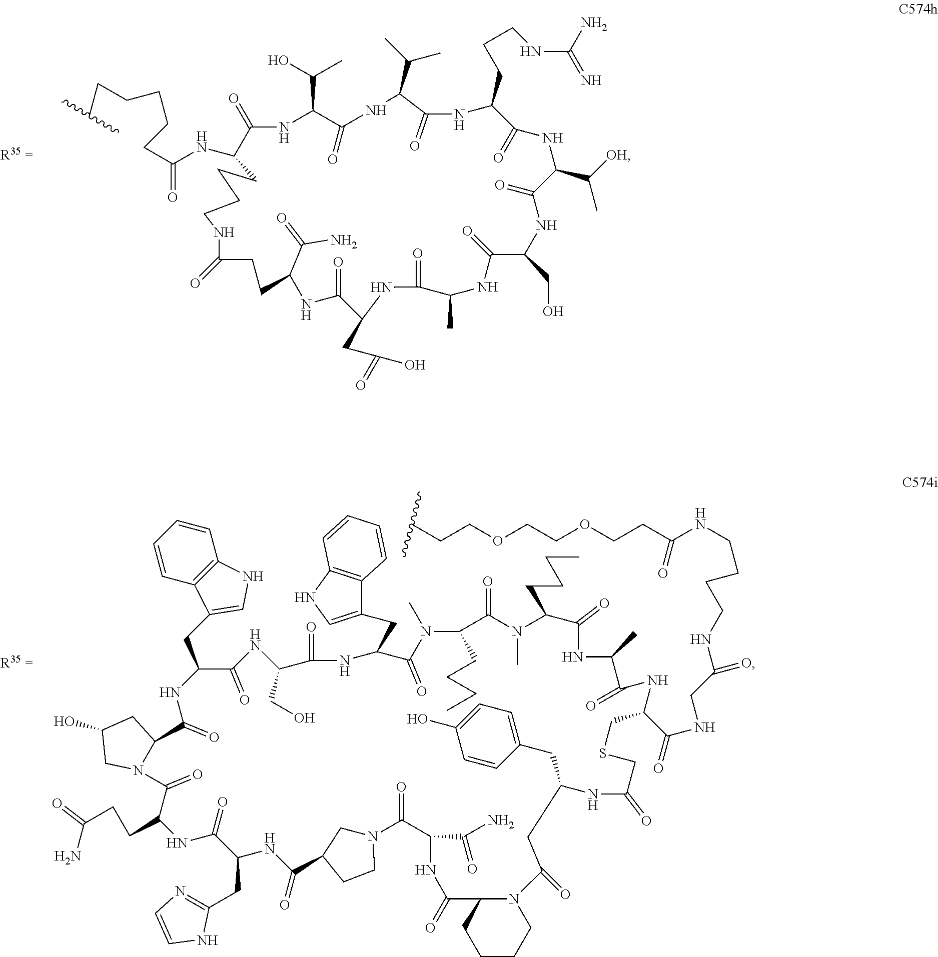 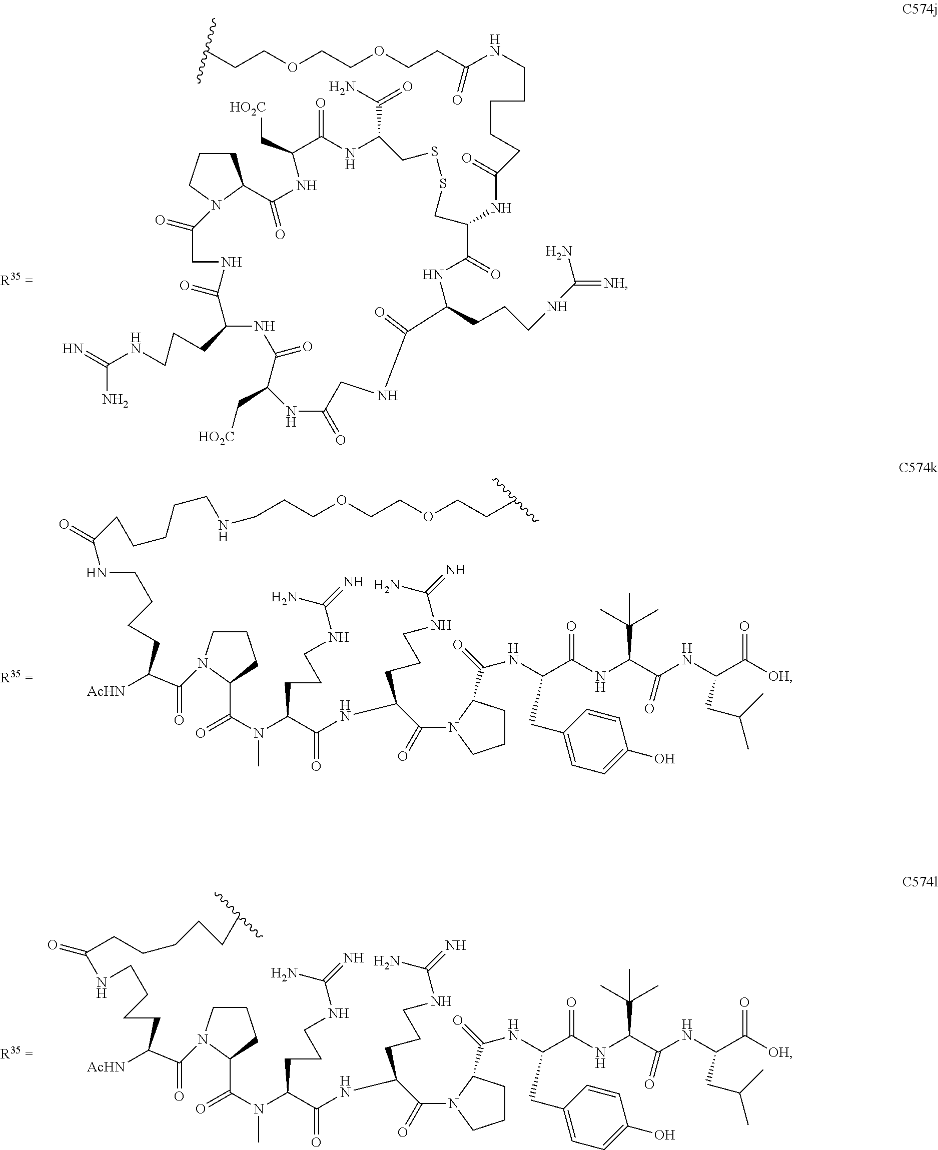 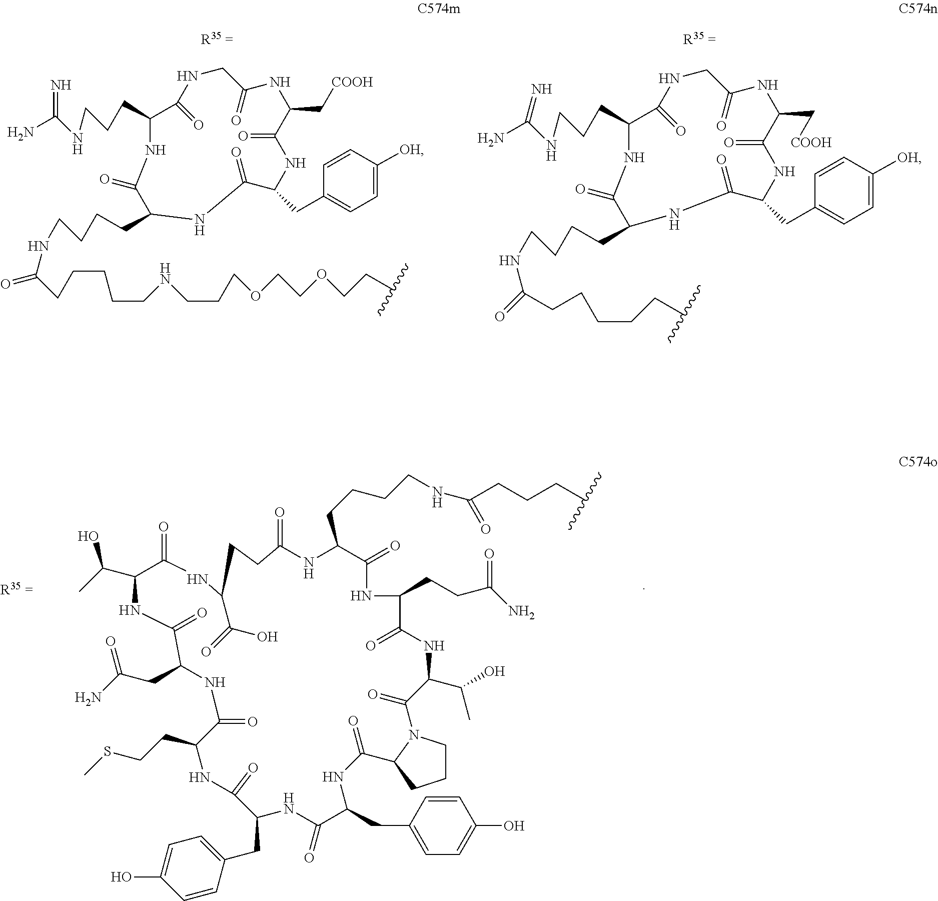 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O] 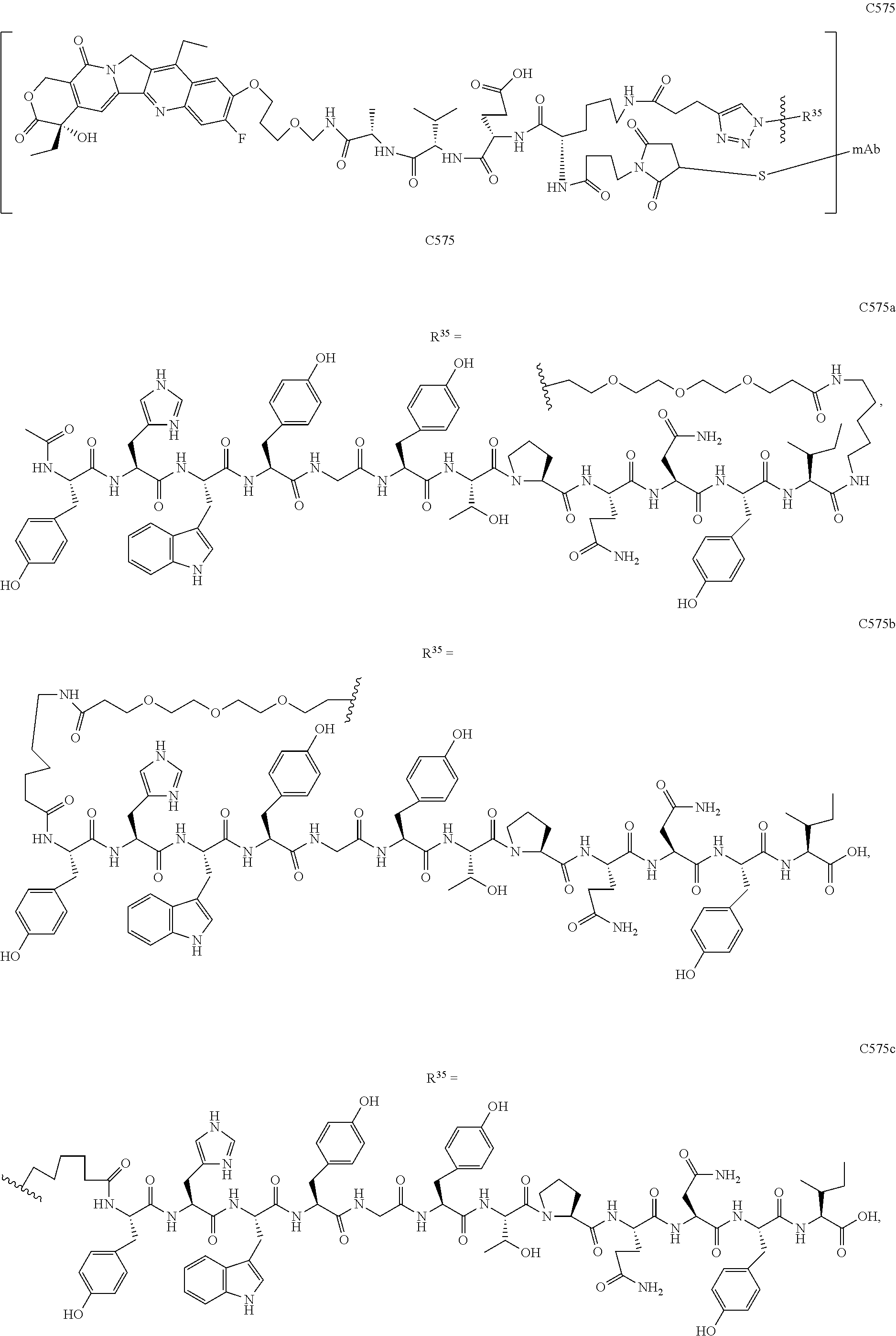 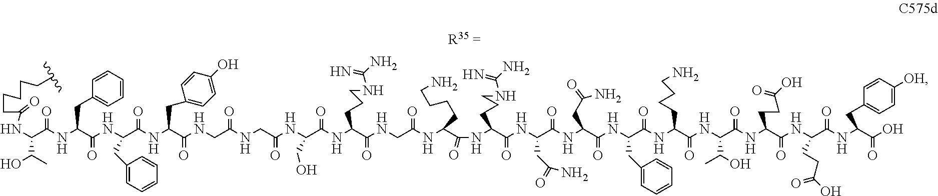 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O] 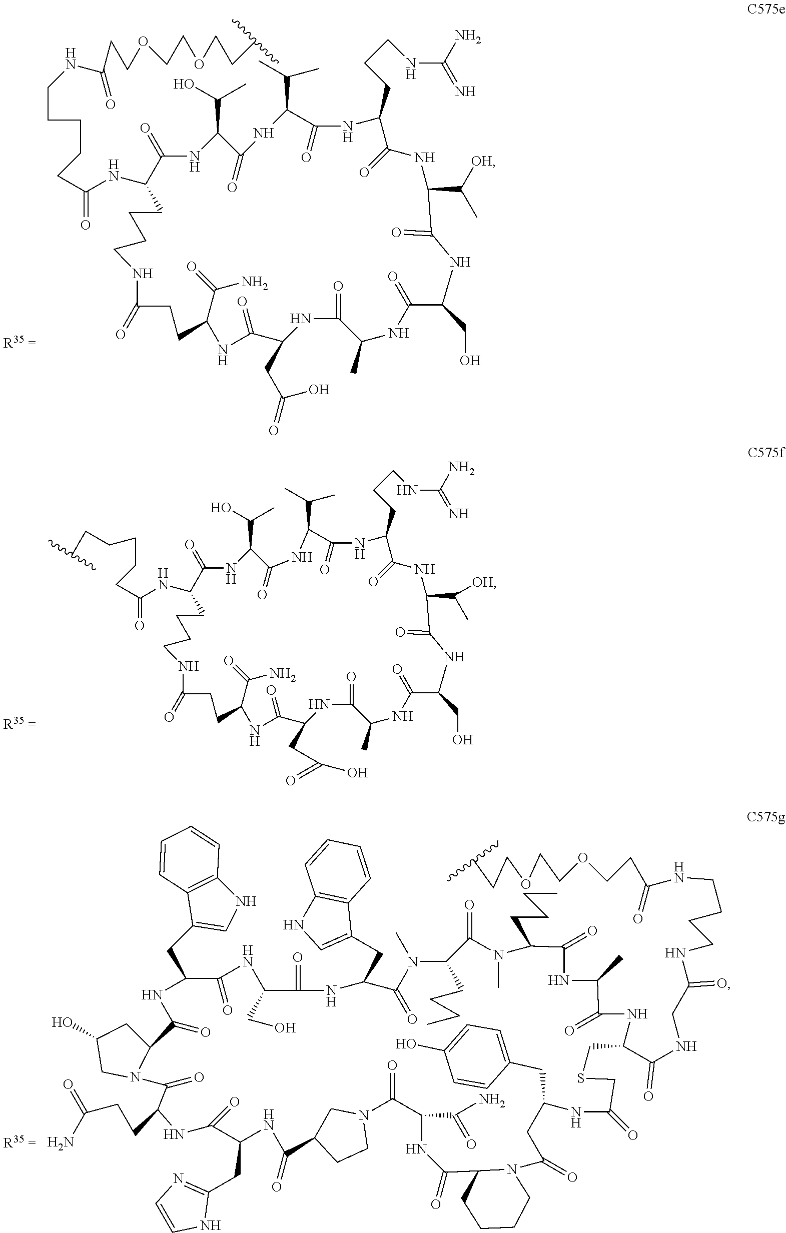 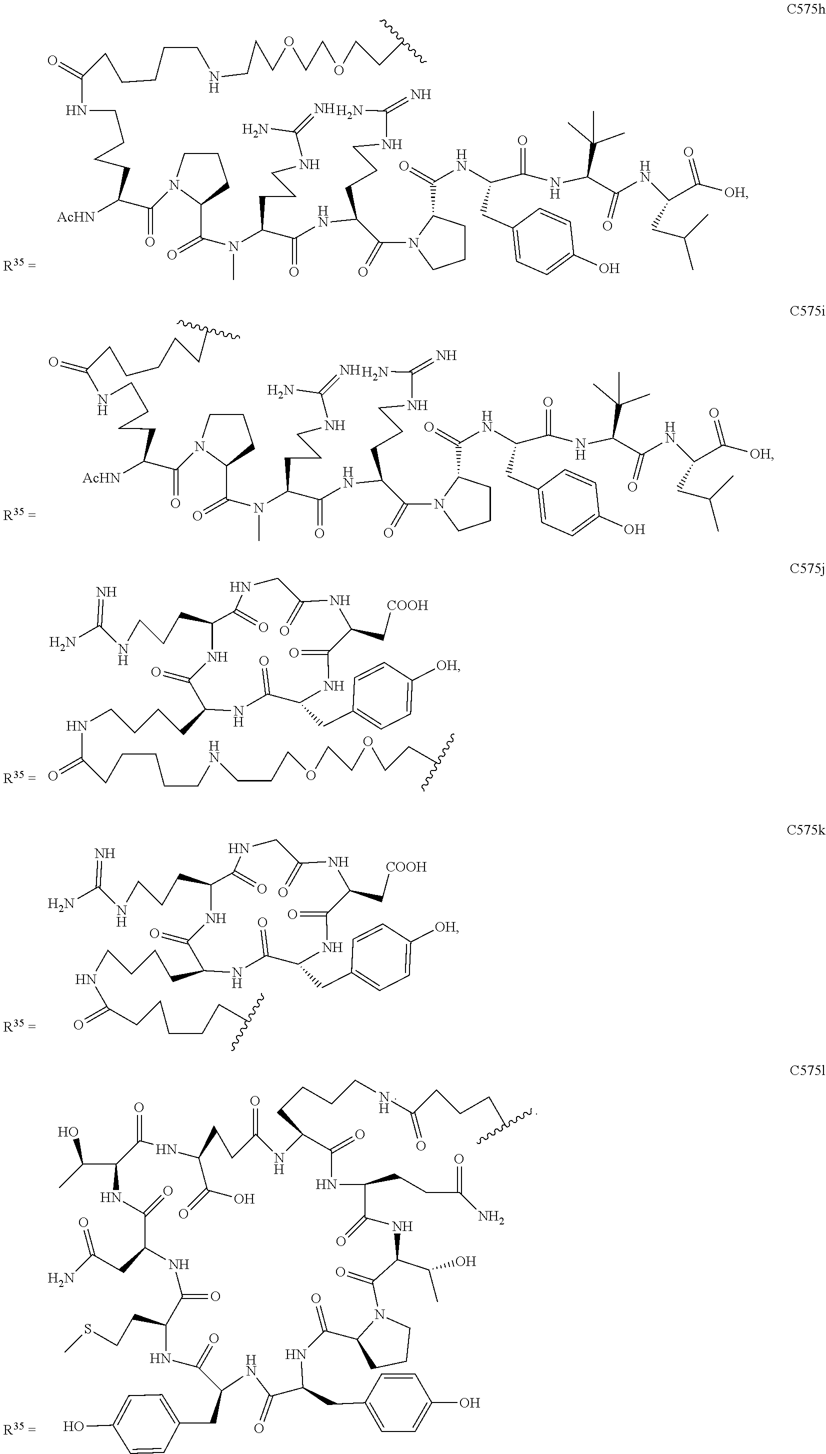 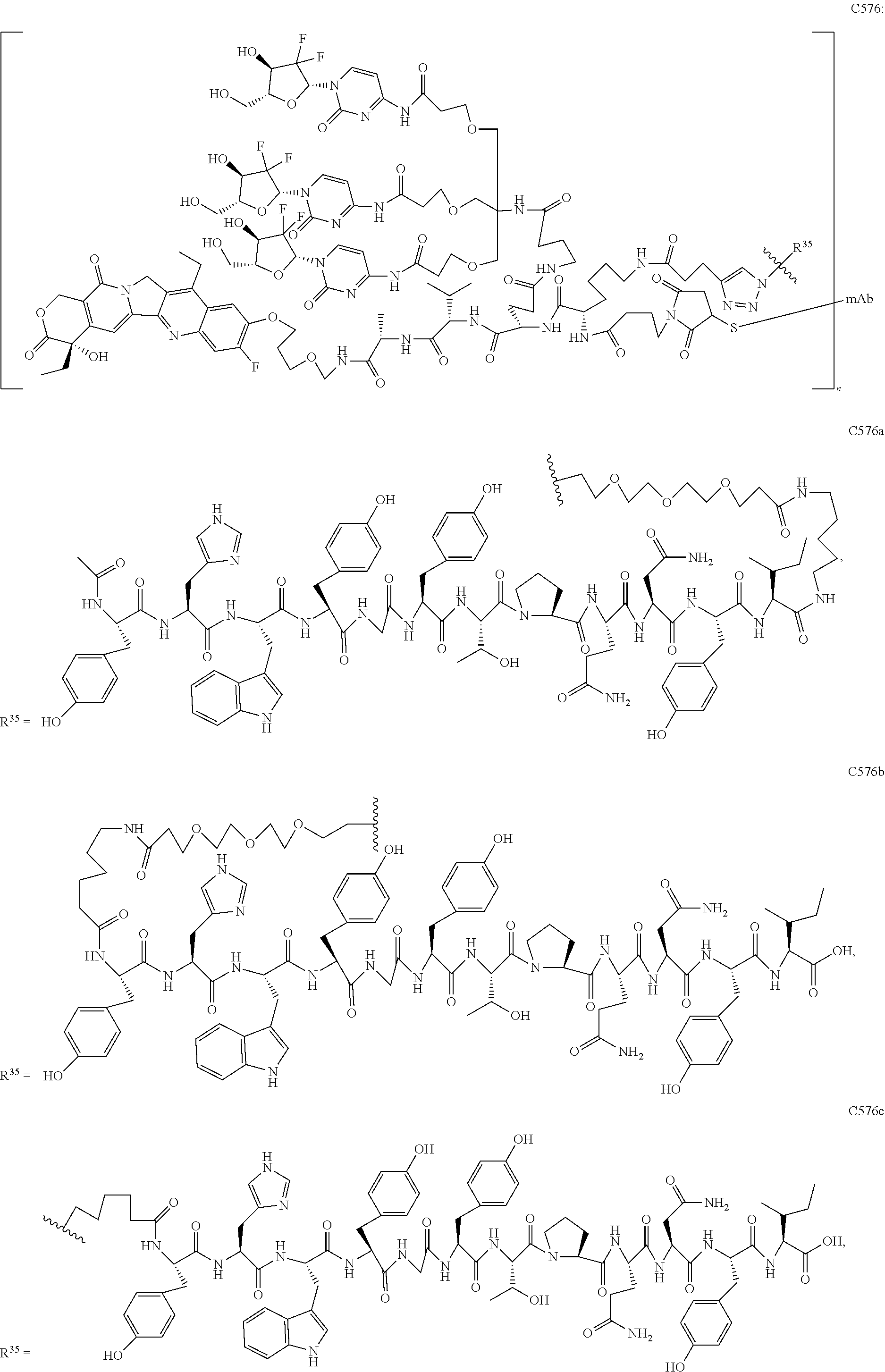 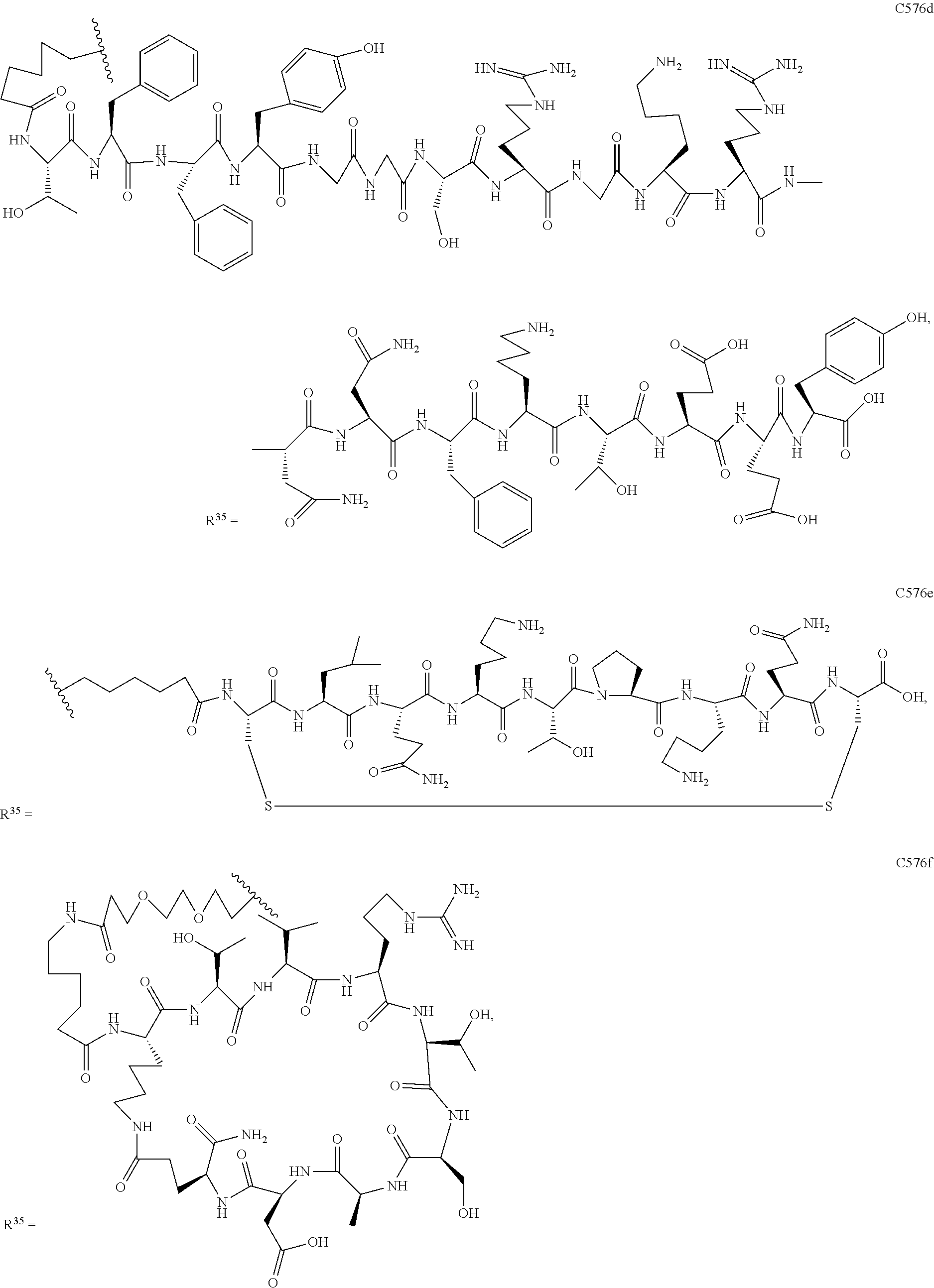 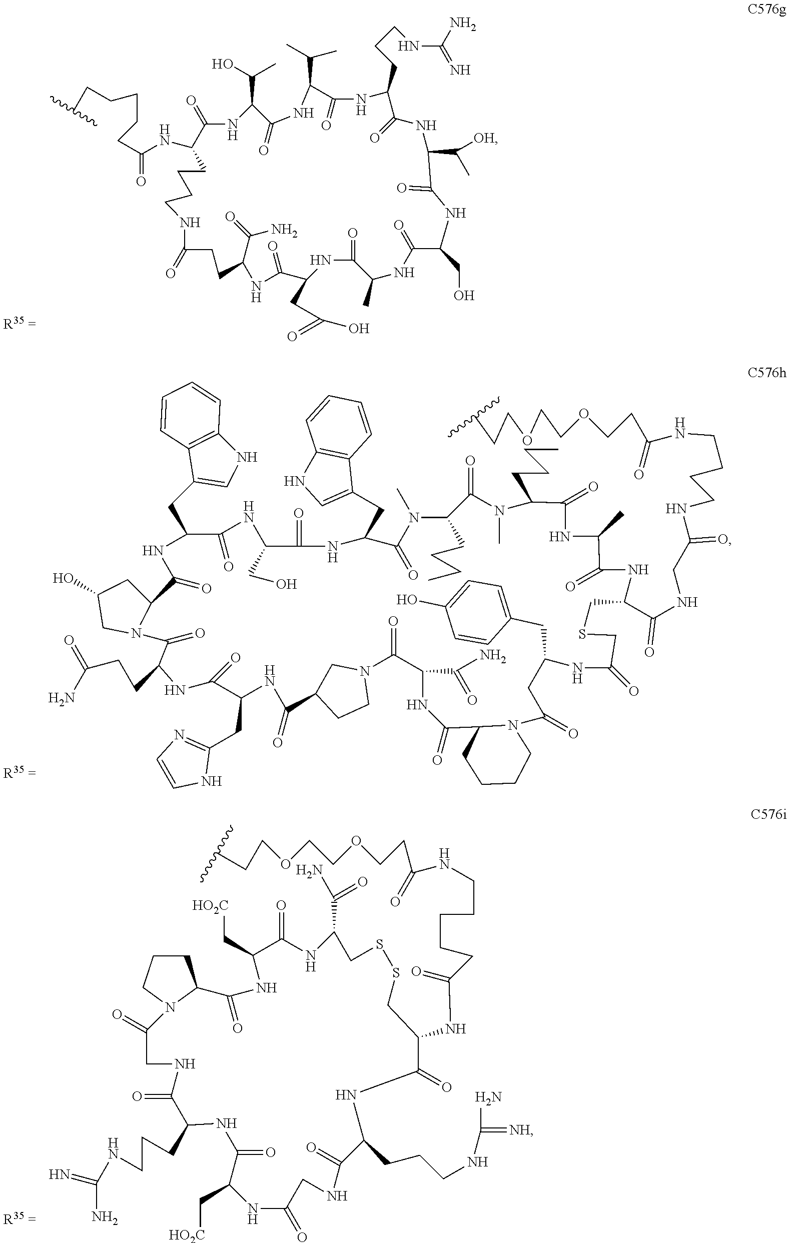 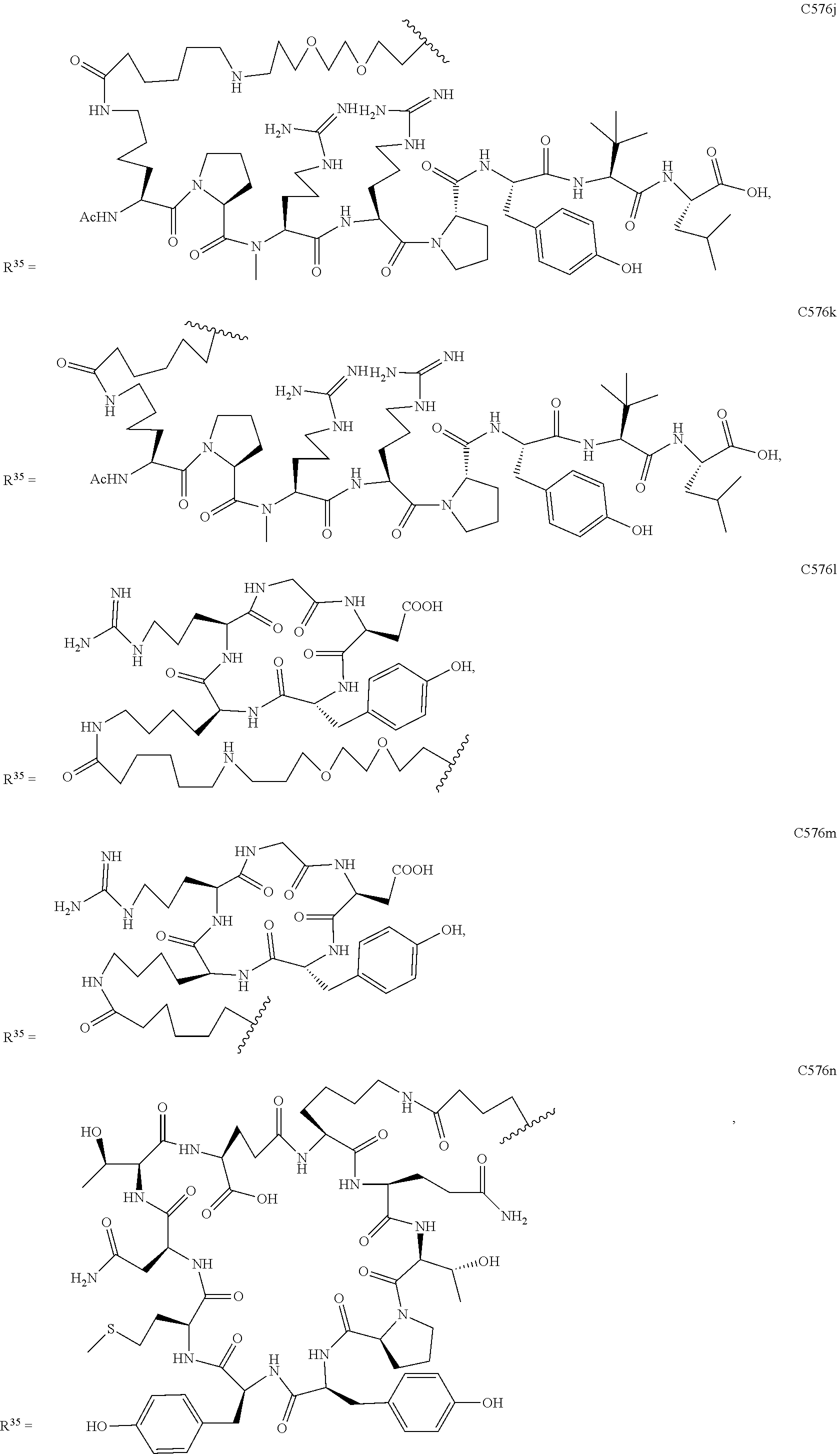 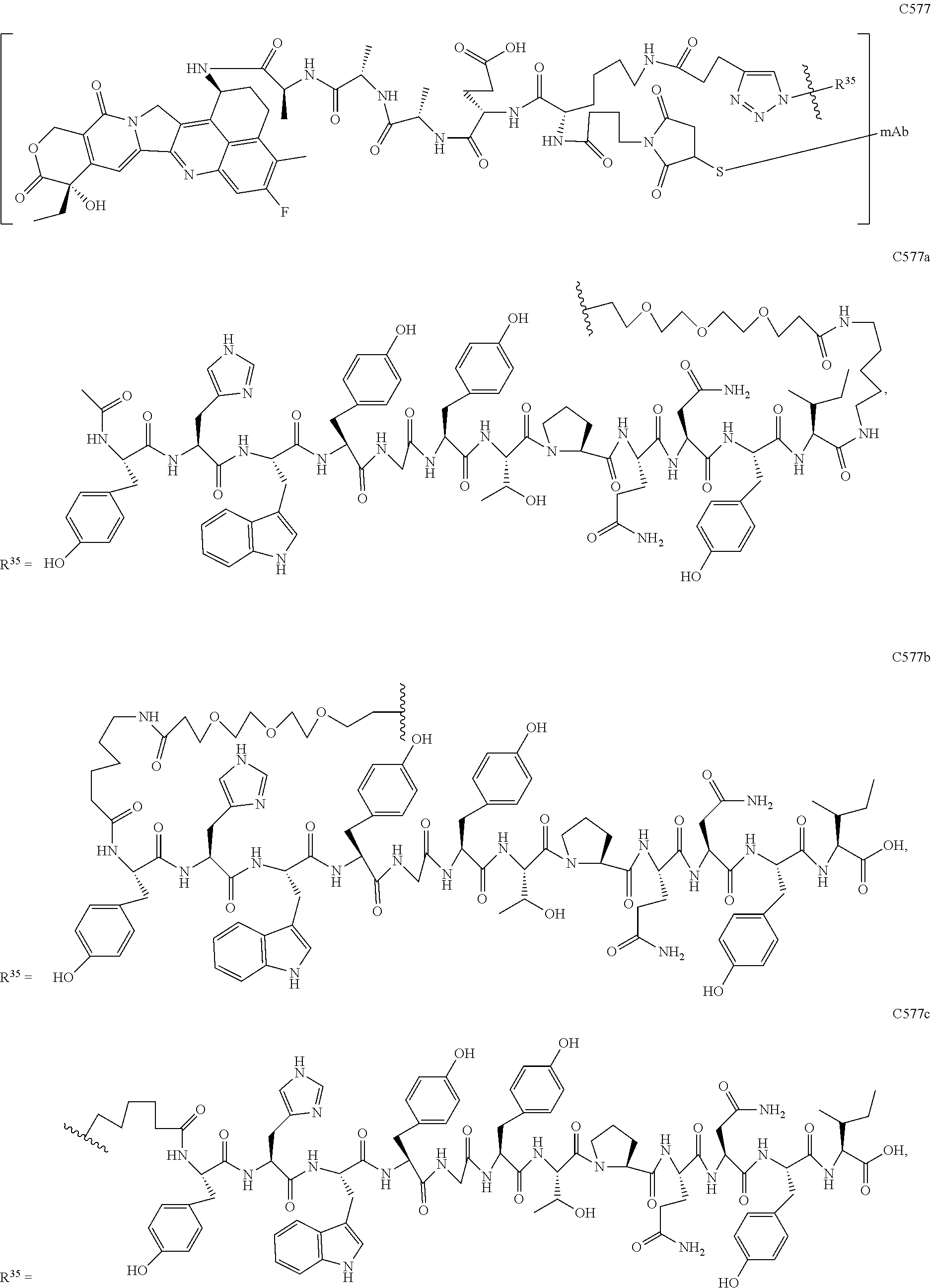 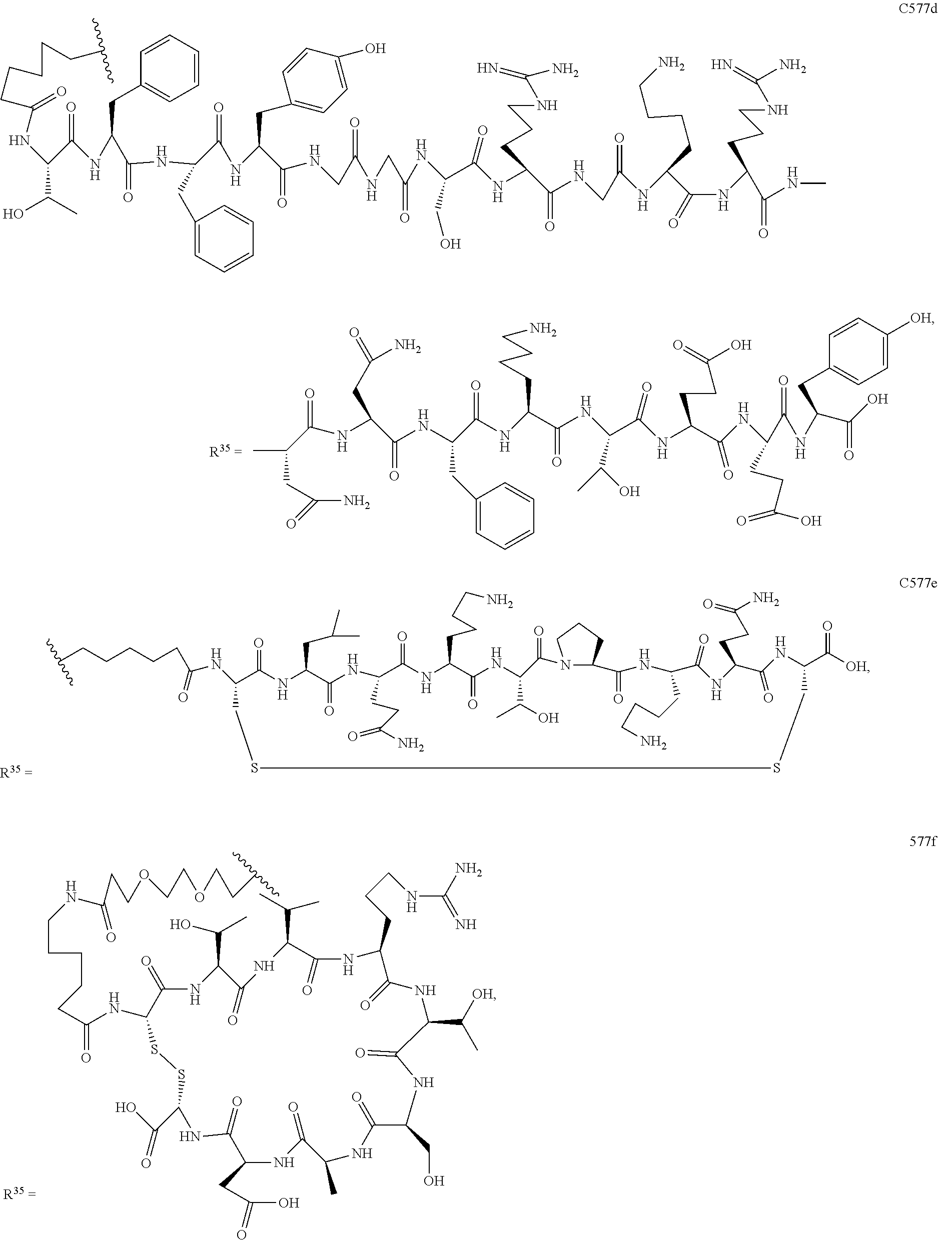 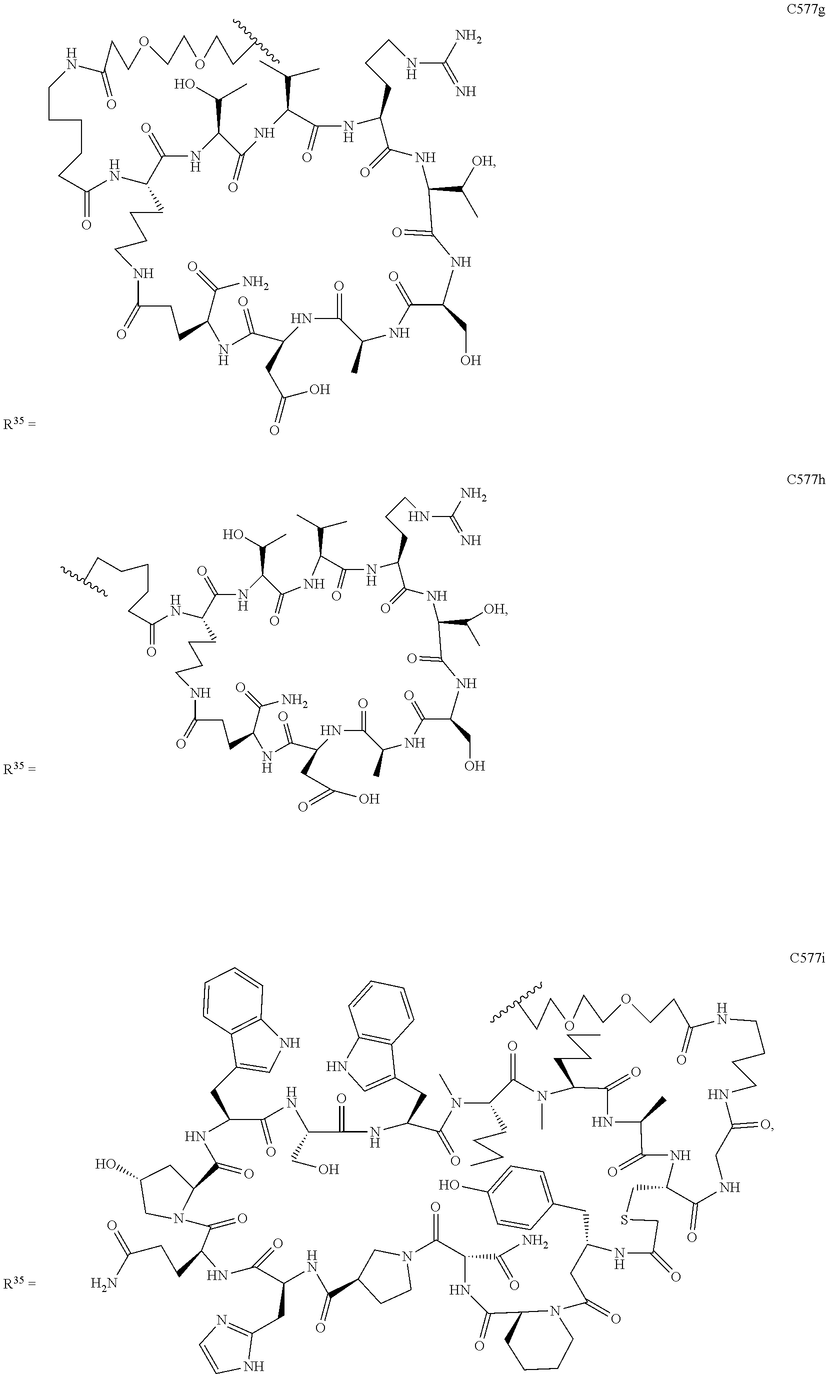 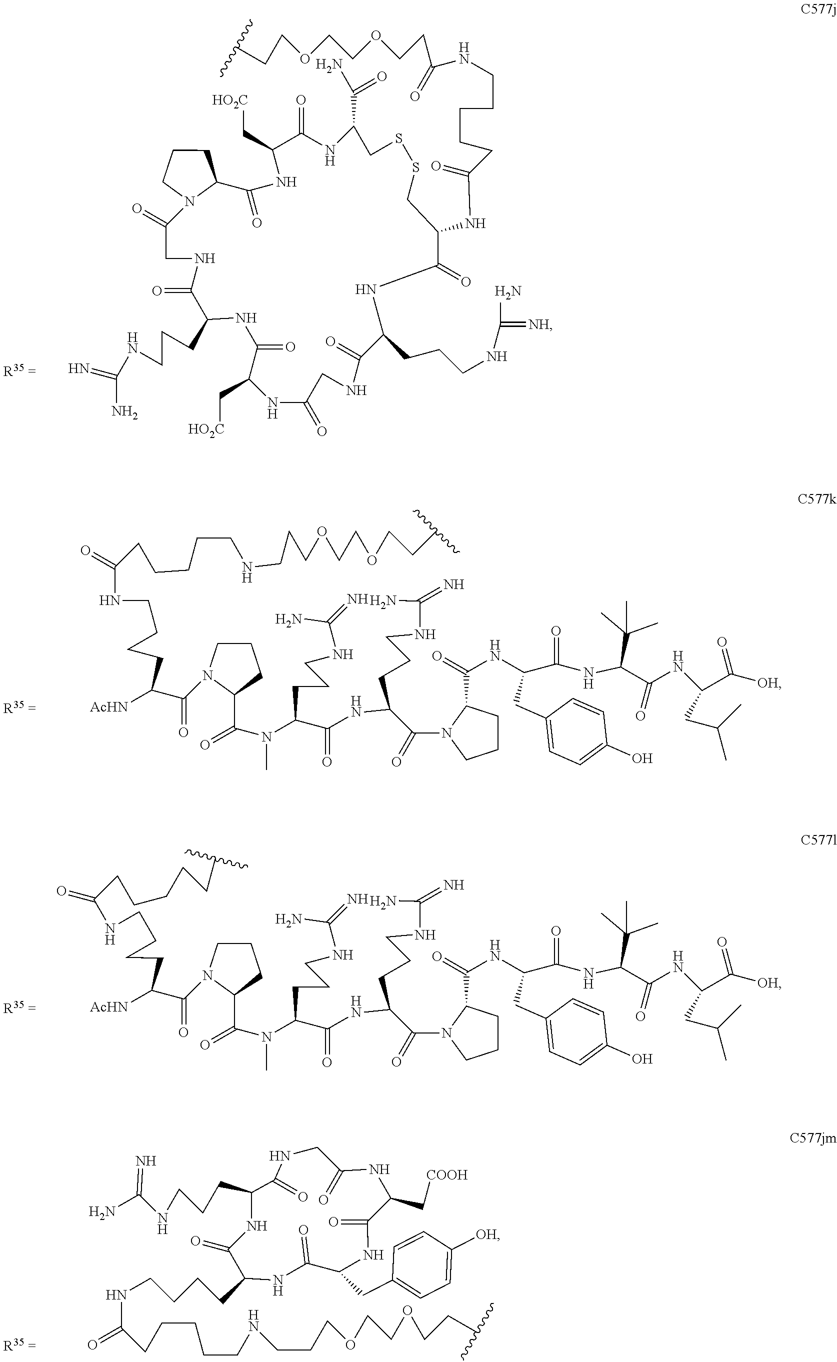 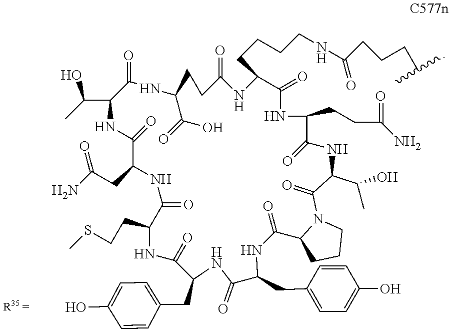 [SMILES: *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O] 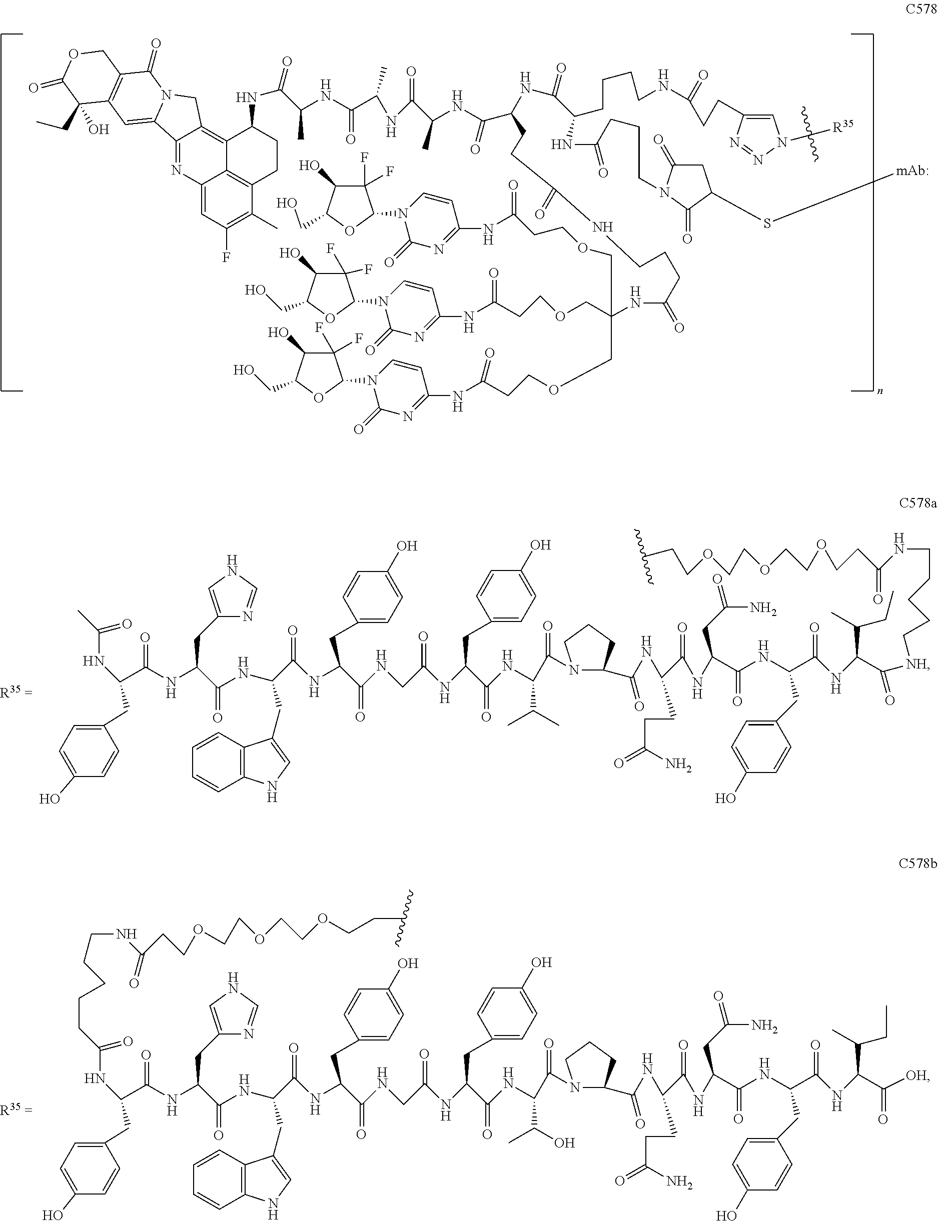 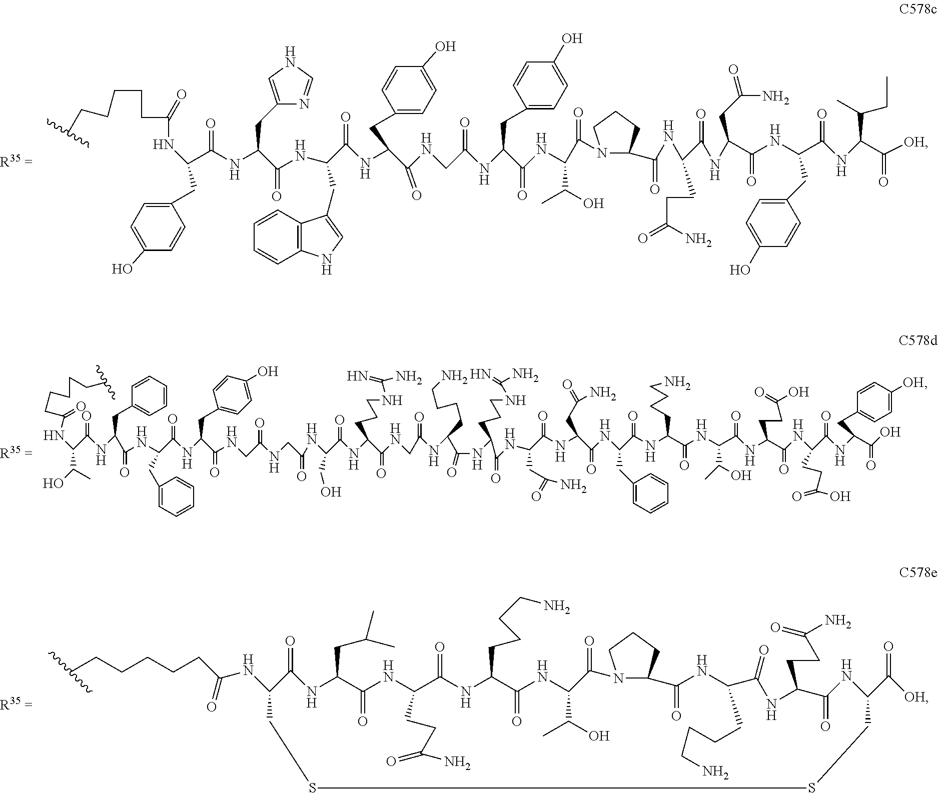 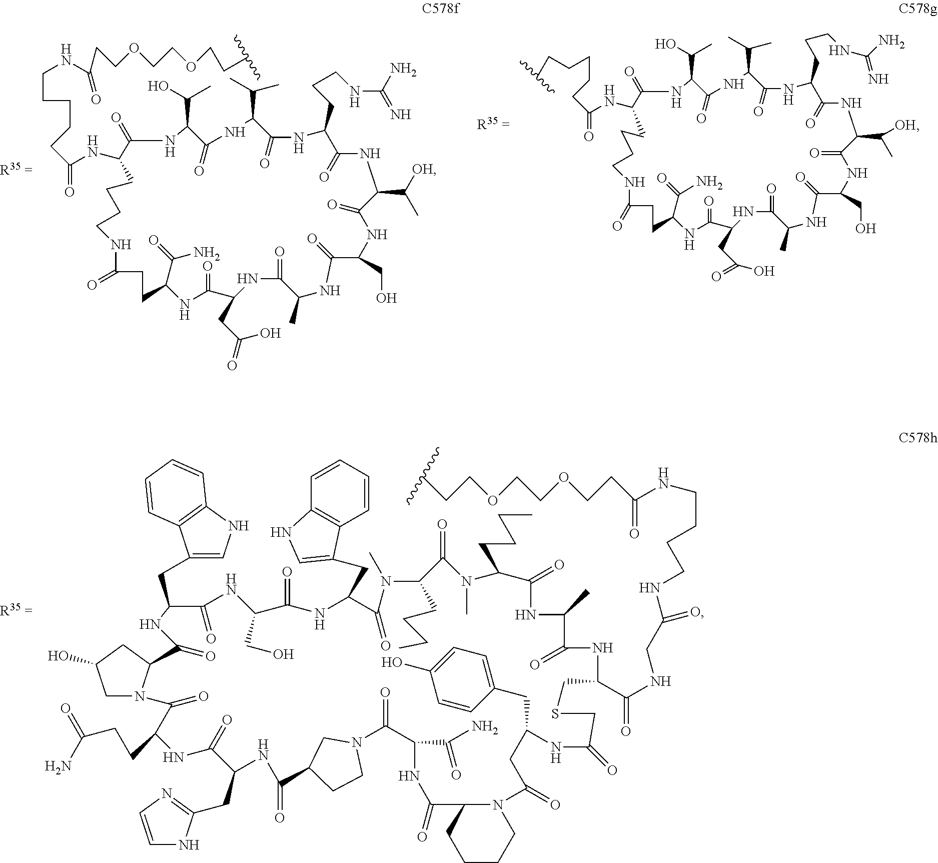 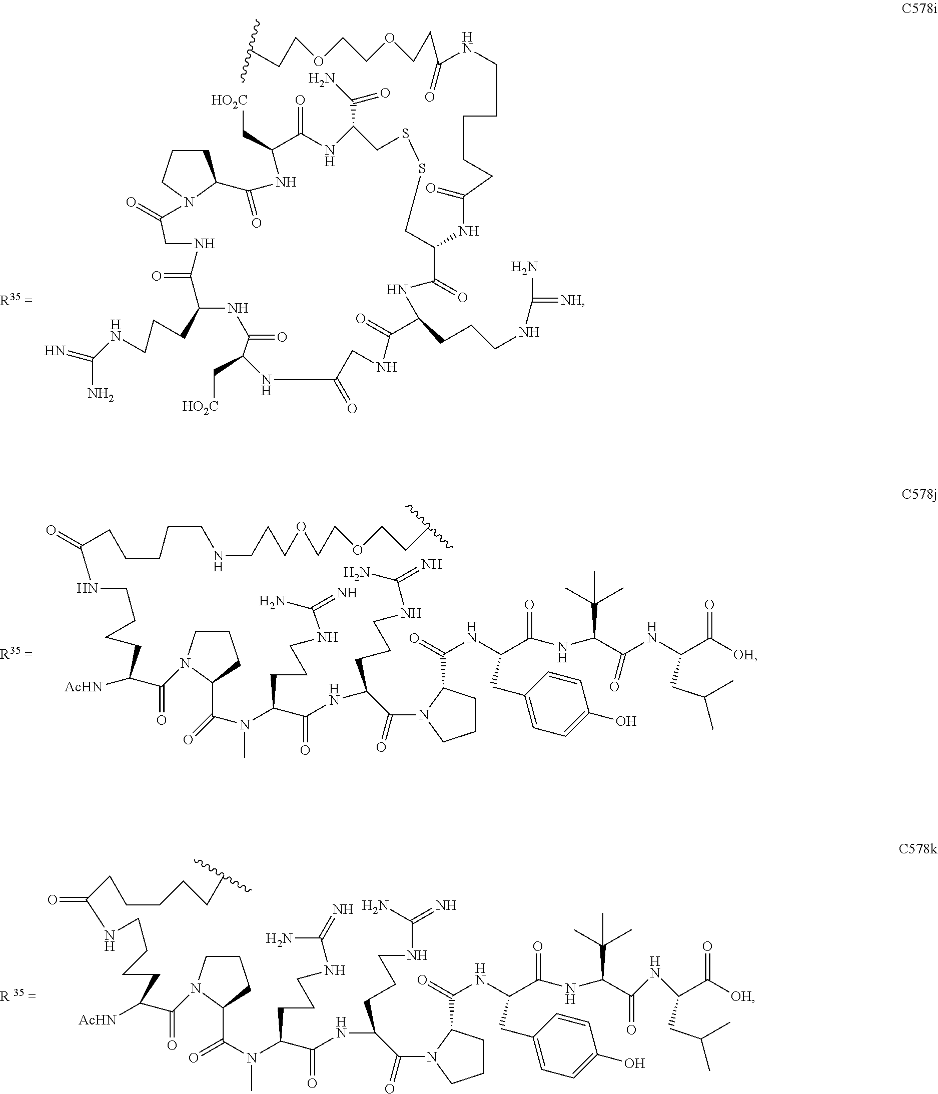 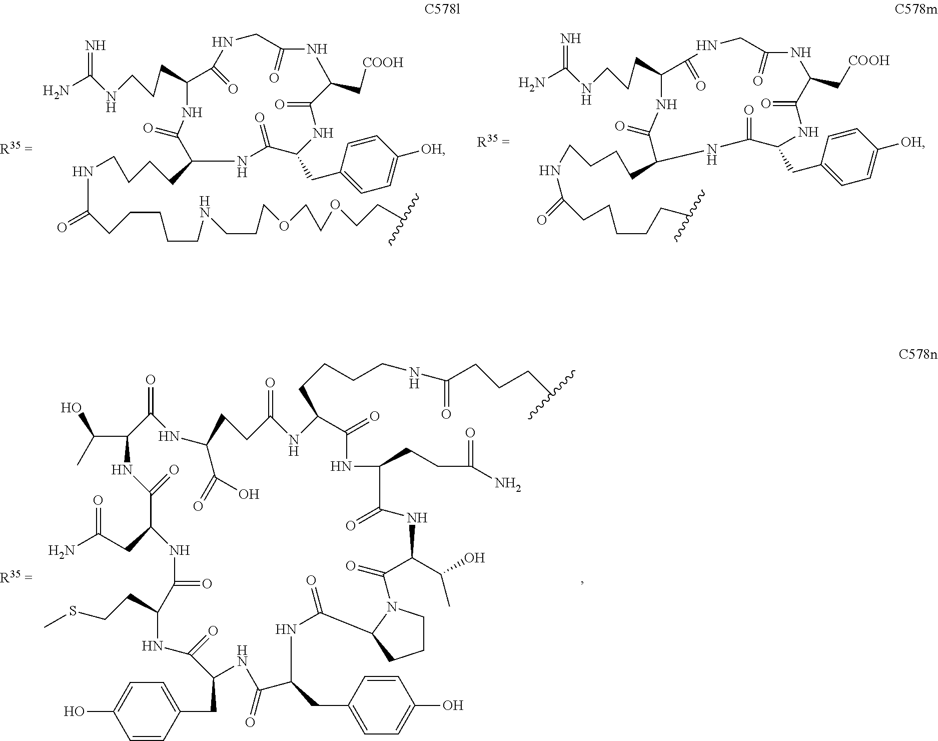 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O] 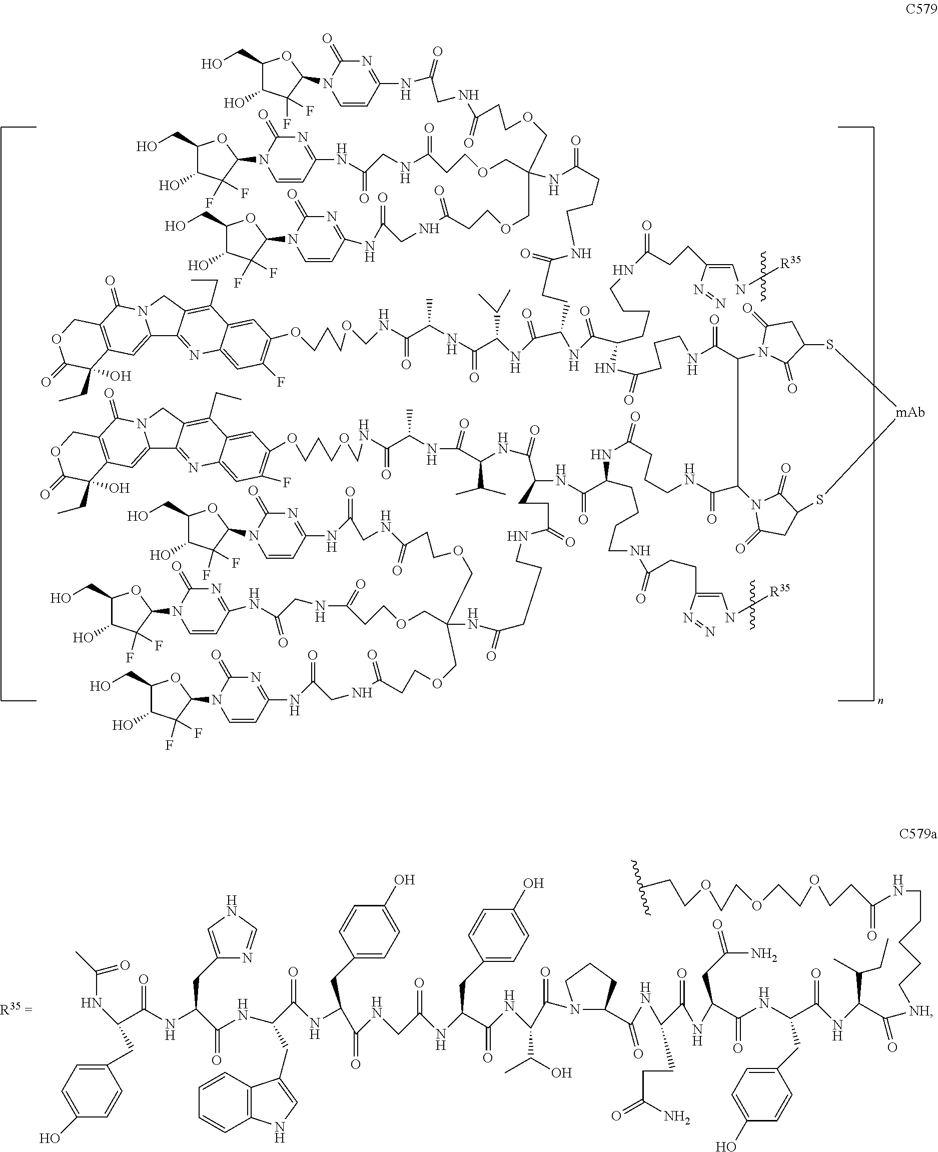 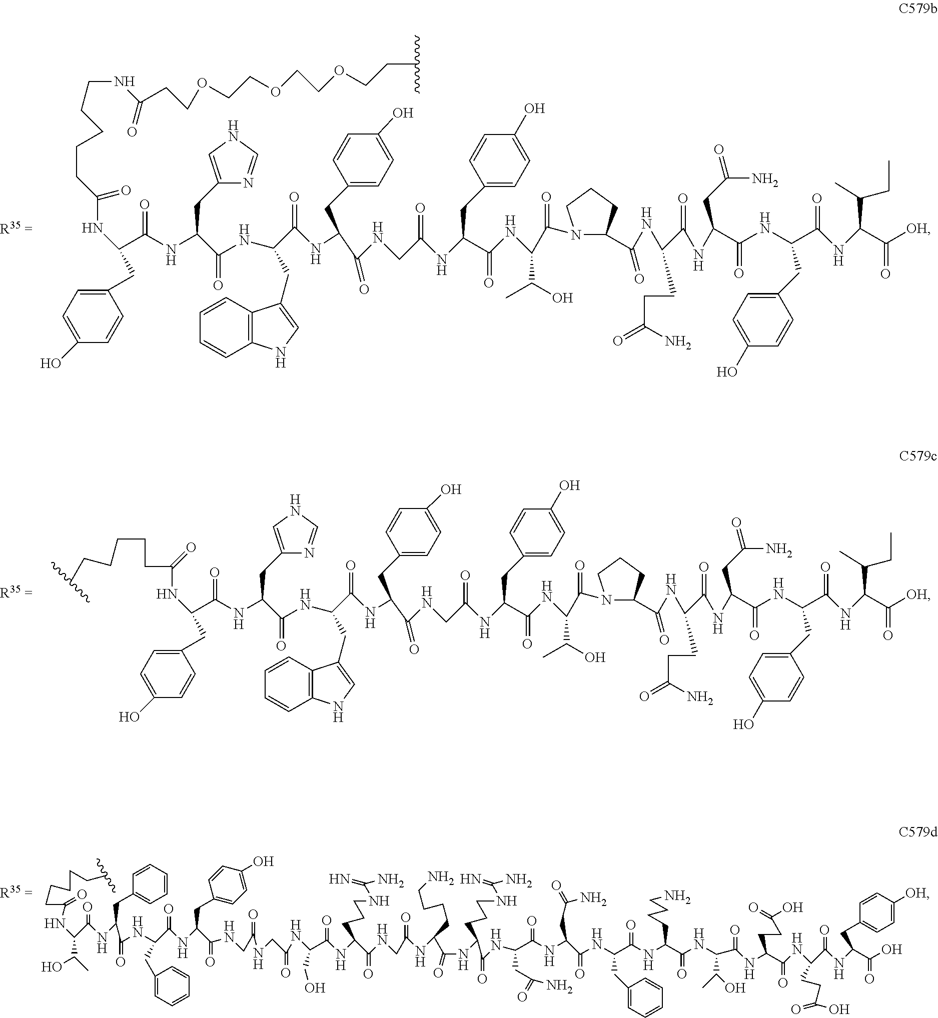 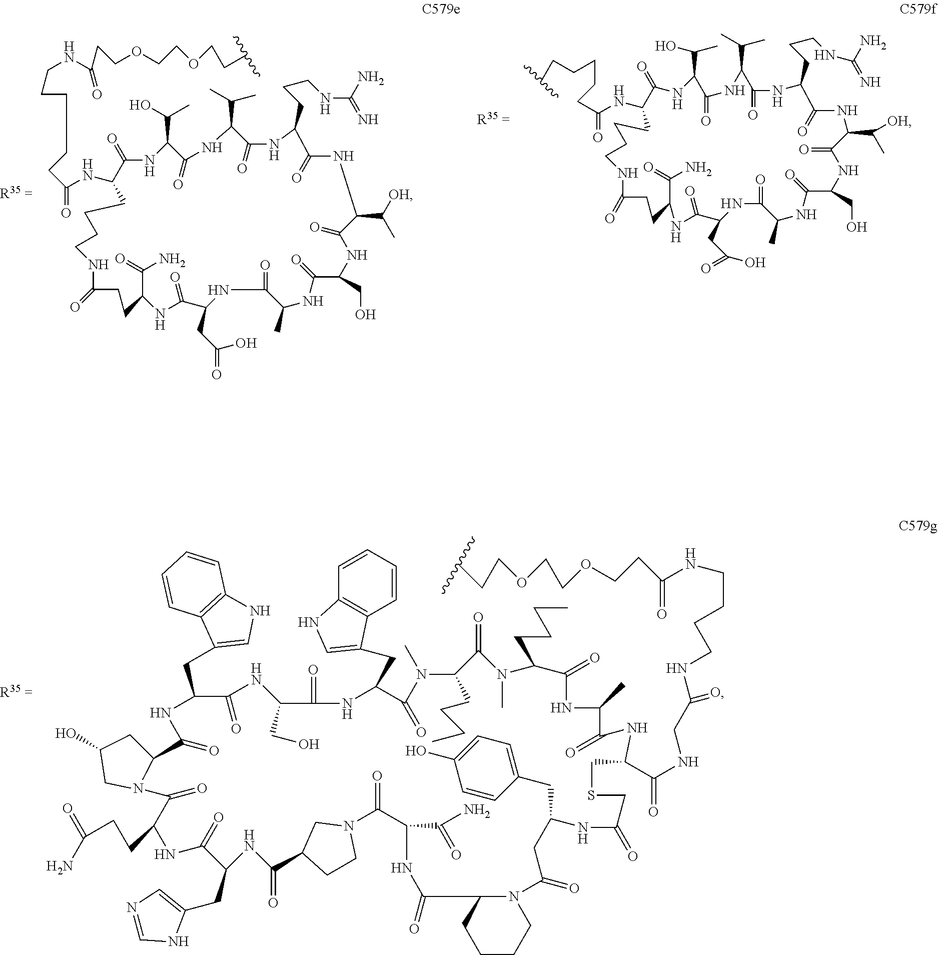 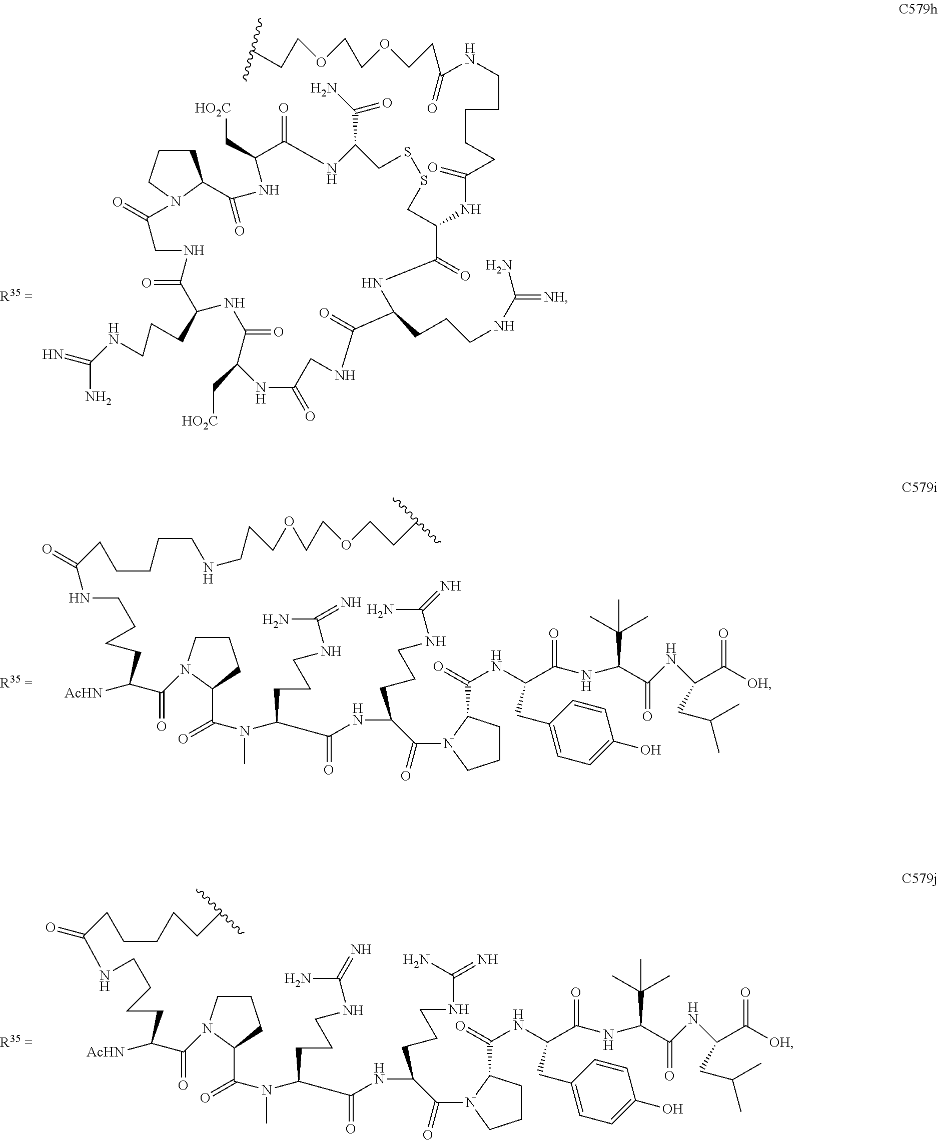 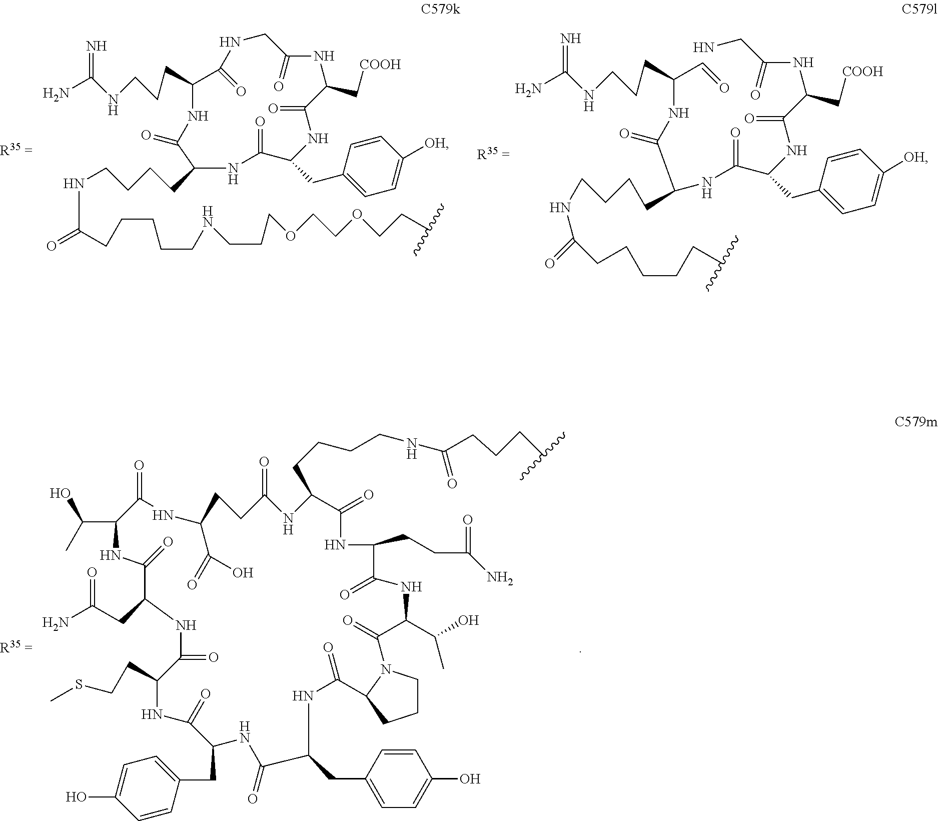 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@H](NC(=O)[C@@H](Cc1ccc(O)cc1)NC(=O)[C@H](CC(=O)O)NC(=O)CN)C(=O)N[C@H](C=O)CCCNC(=N)N ; *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O] 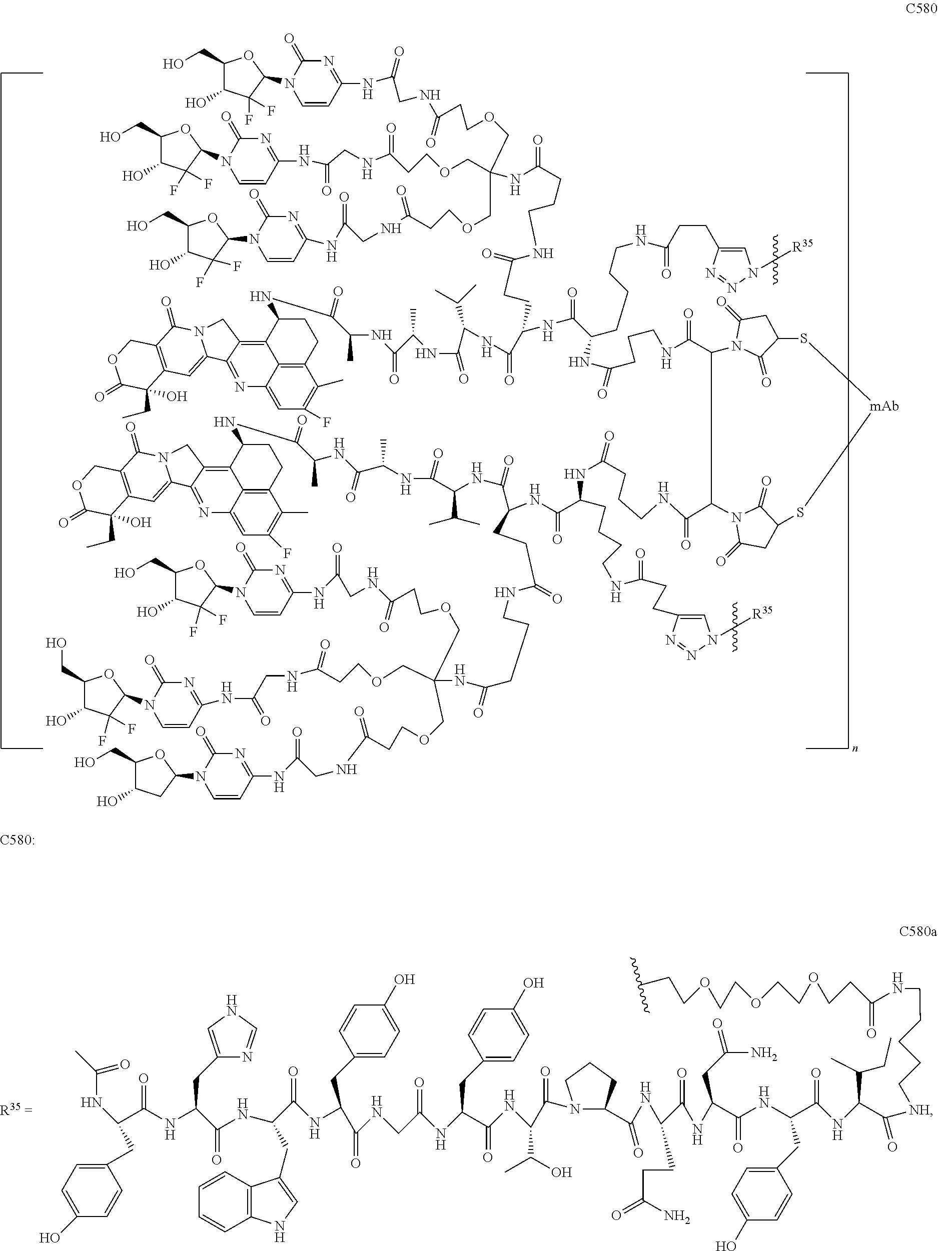 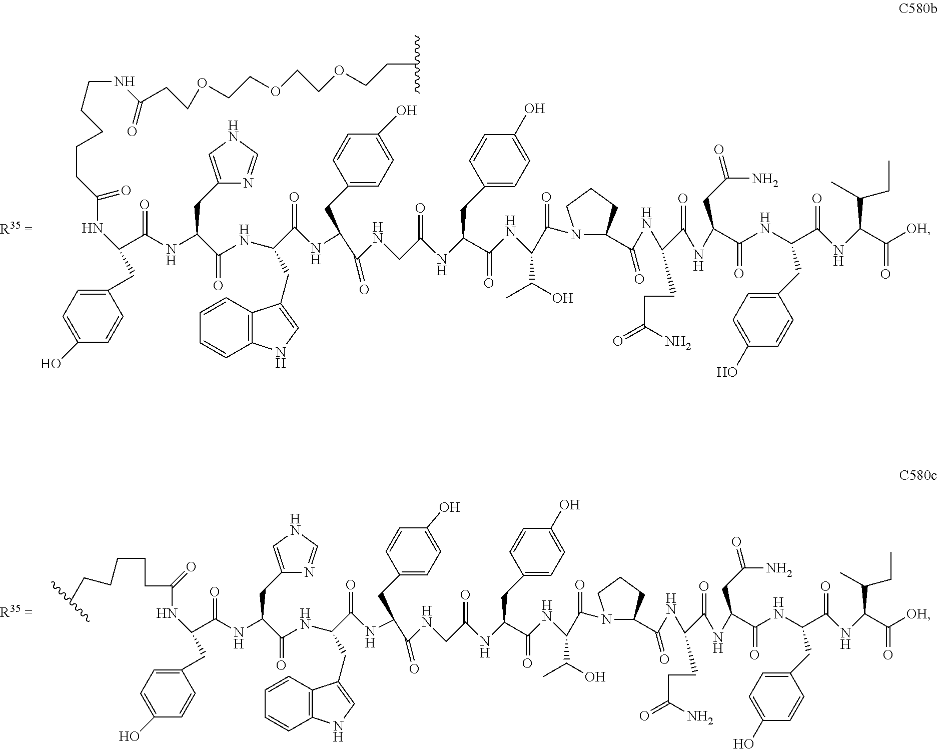 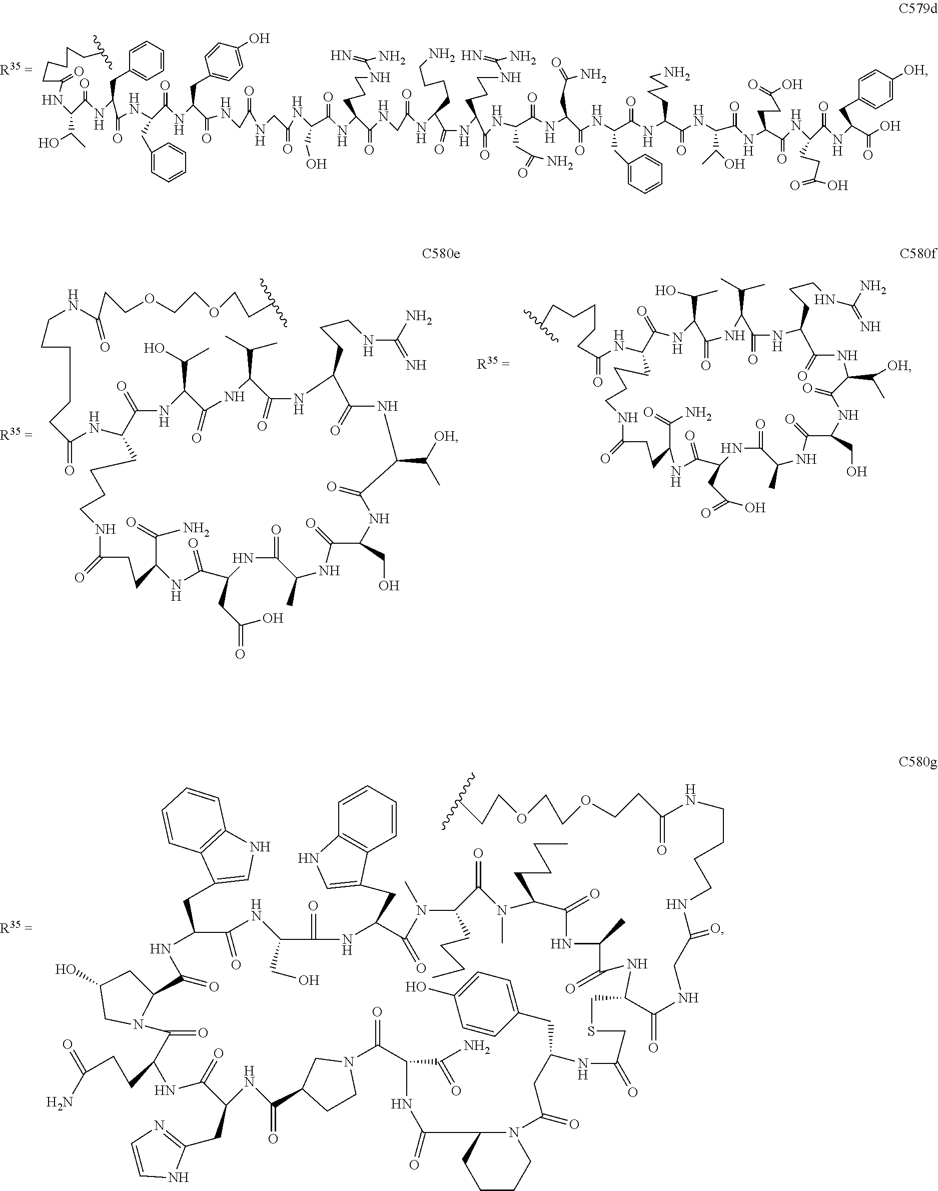 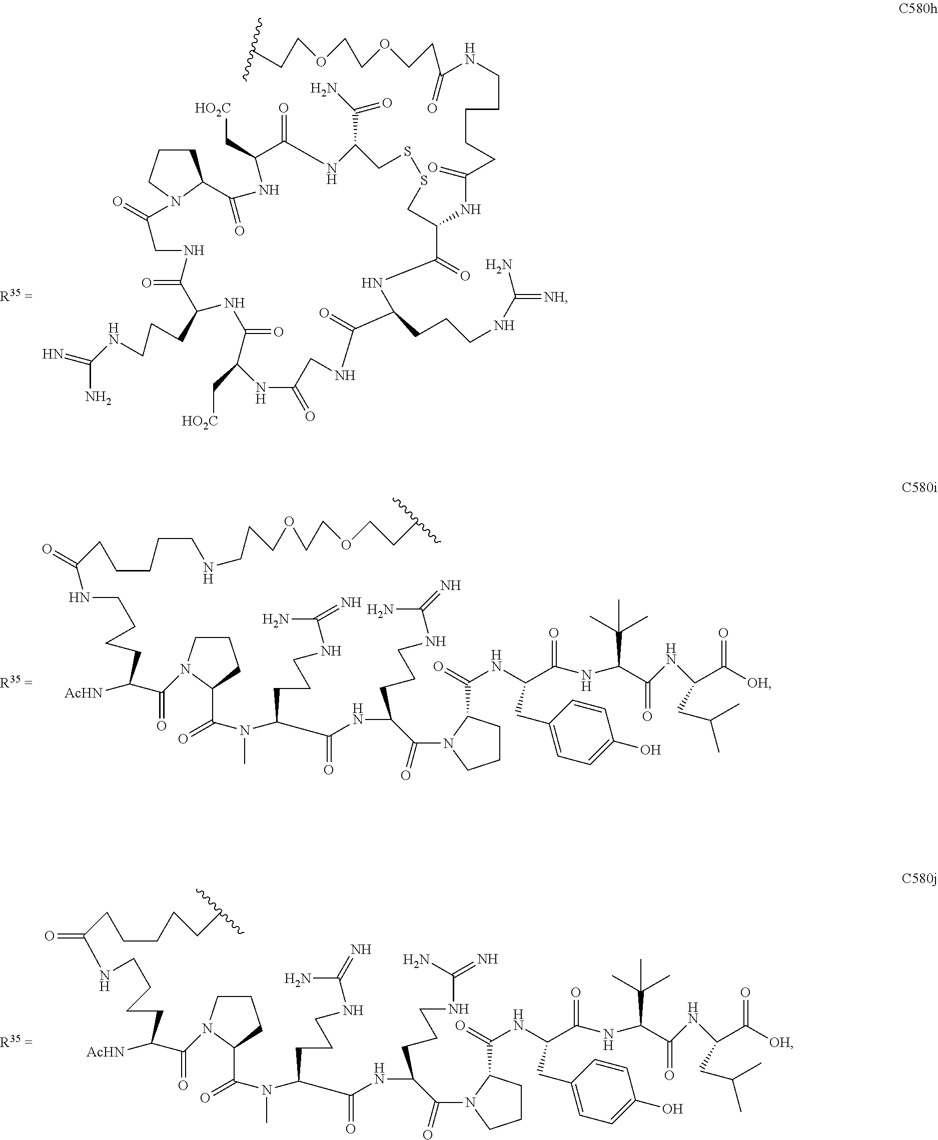 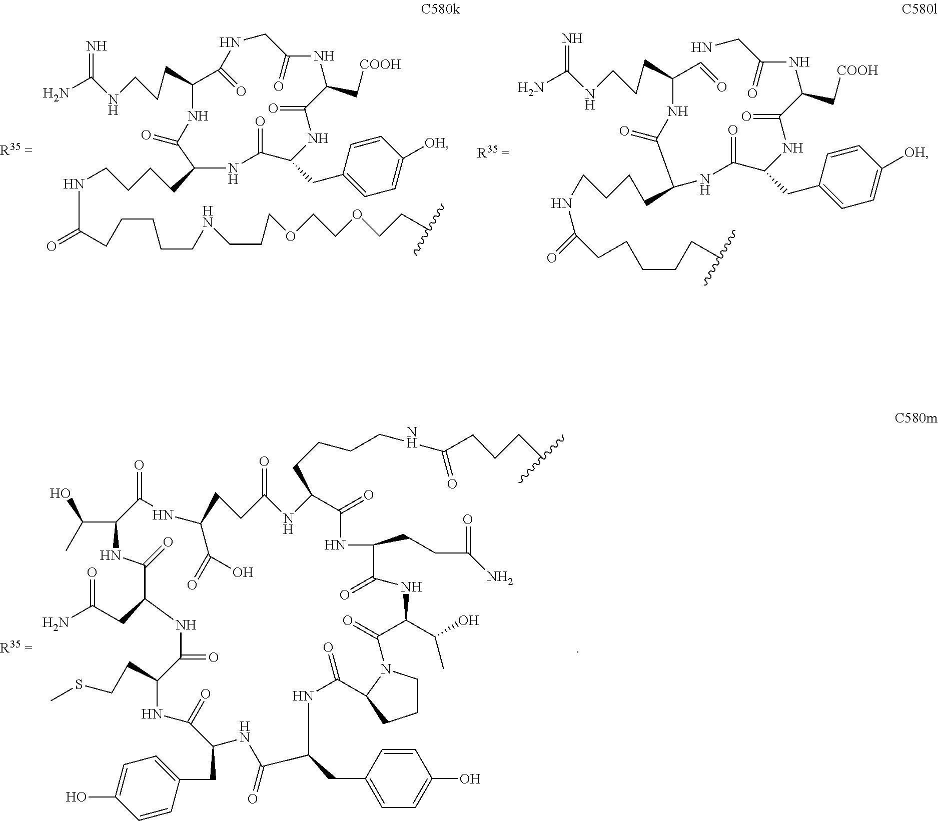 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@H](NC(=O)[C@@H](Cc1ccc(O)cc1)NC(=O)[C@H](CC(=O)O)NC(=O)CN)C(=O)N[C@H](C=O)CCCNC(=N)N ; *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O] 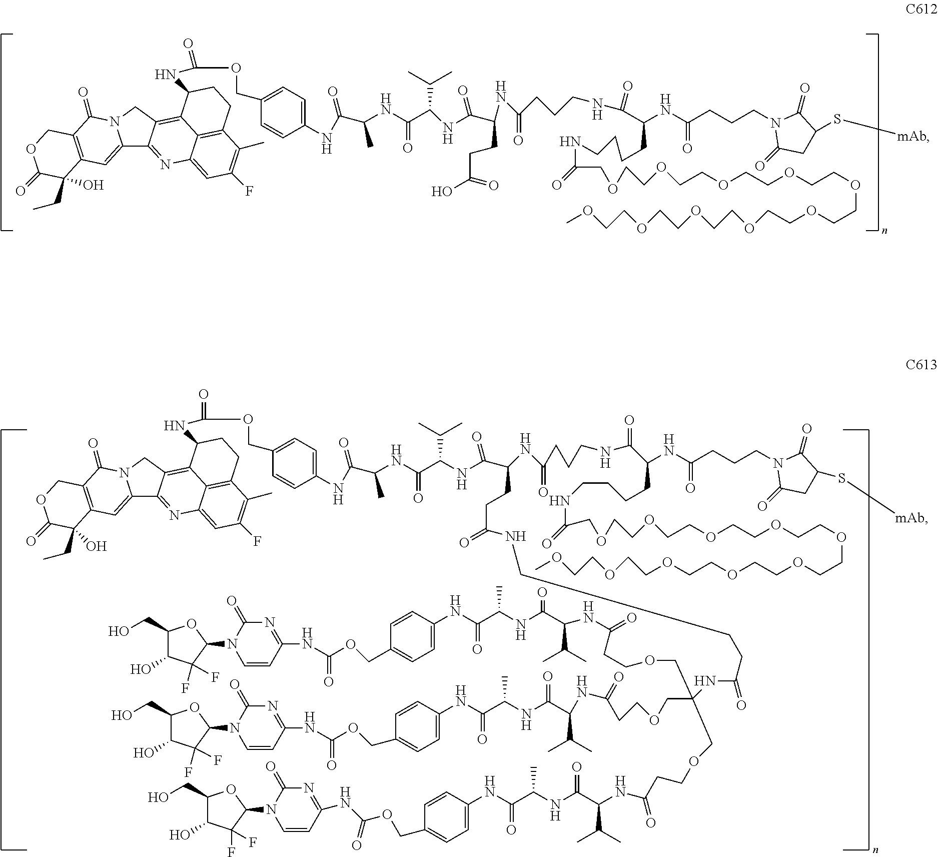 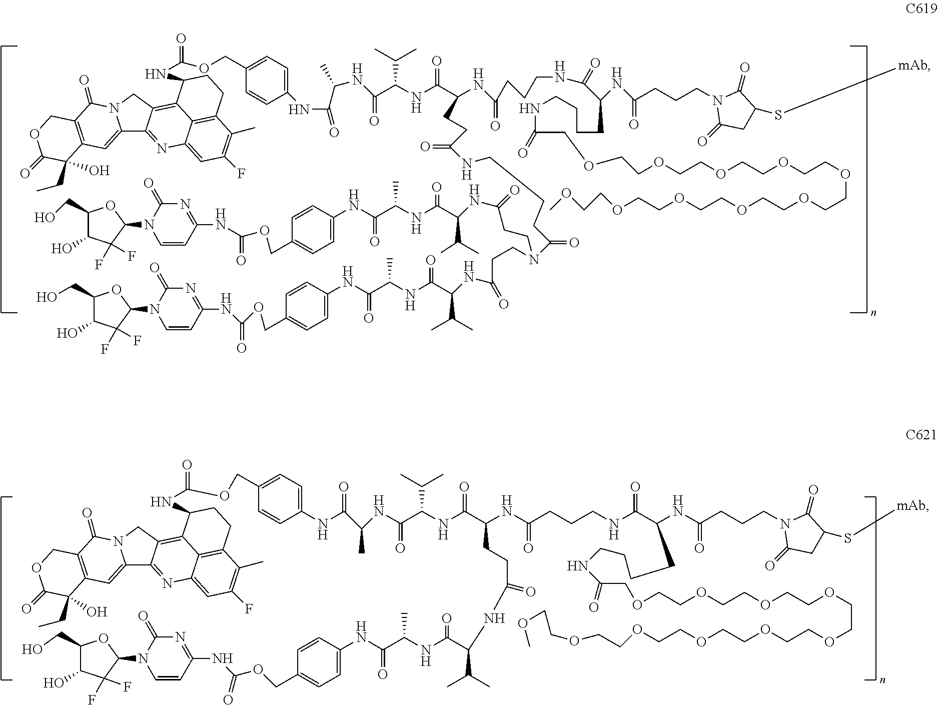 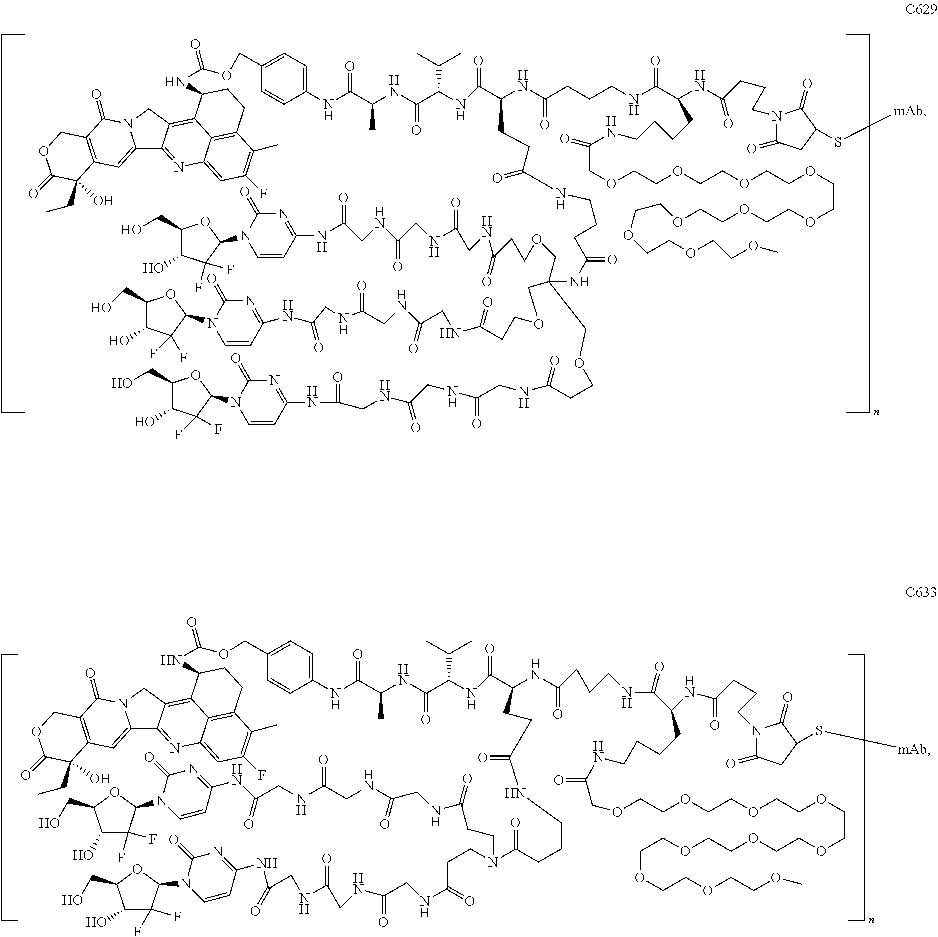 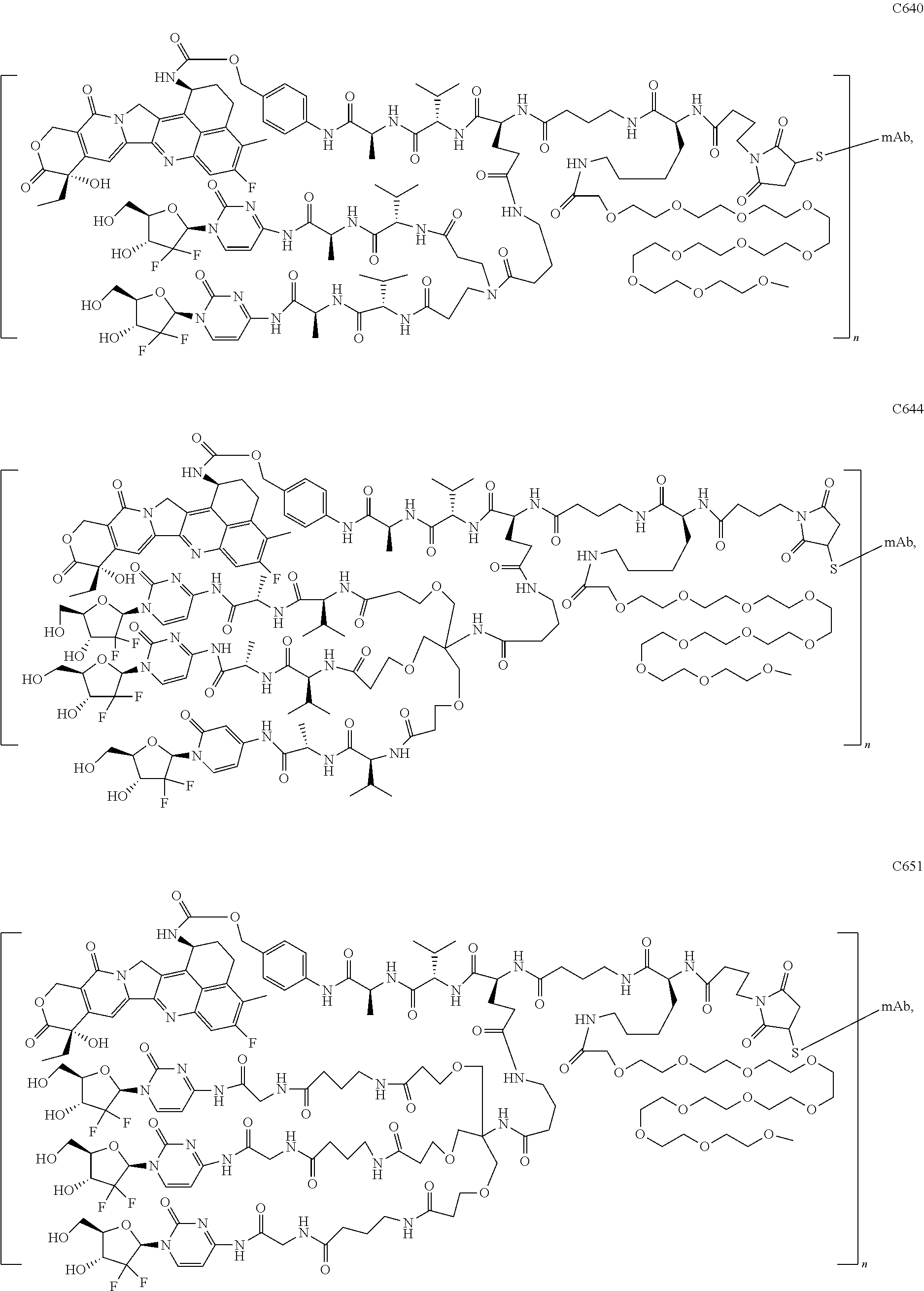 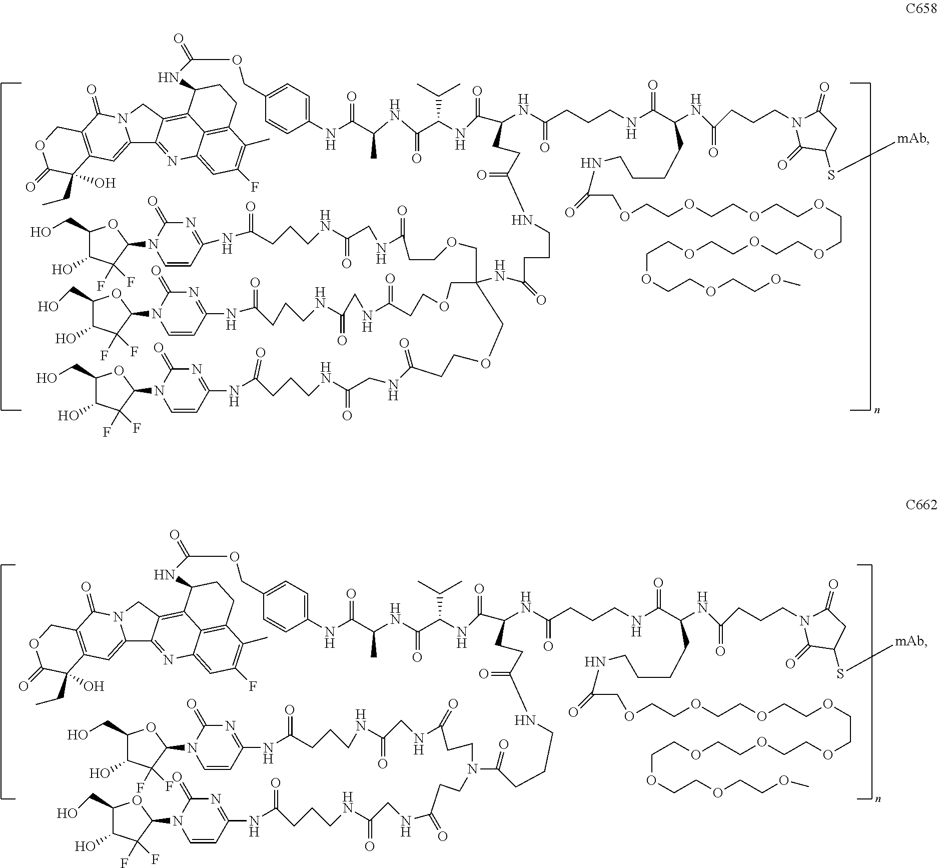 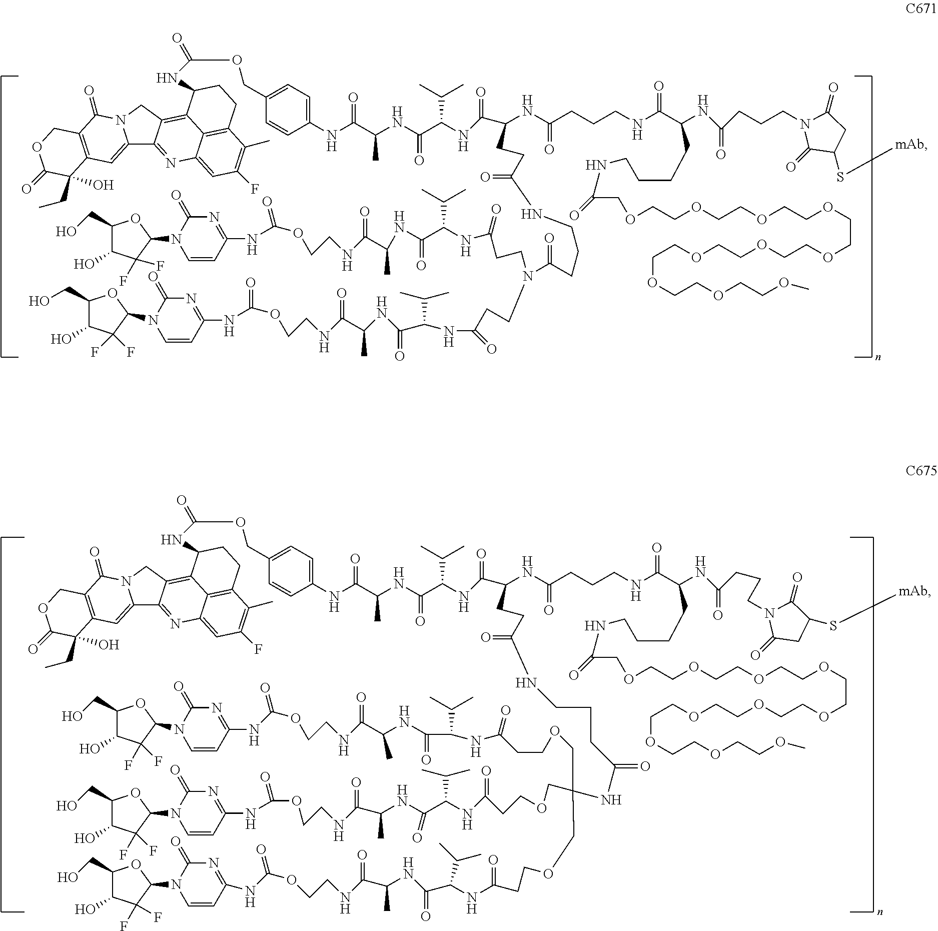 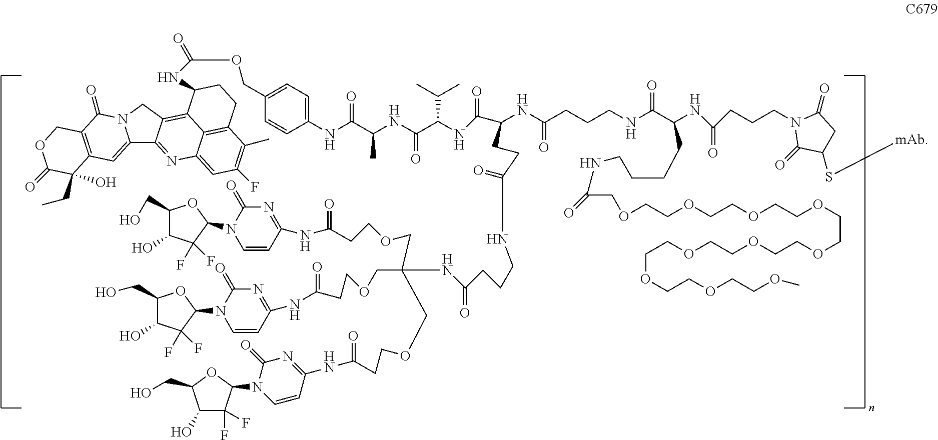
wherein mAb above is an antibody; n is 1~30.

10. The antibody drug conjugate according to claim 1, wherein th conjugates of Formula (I), (II), (III) and (IV), are prepared via conjugation reaction of the antibody with a compound having the following Formula (V), (VI) and (VII) respectively: 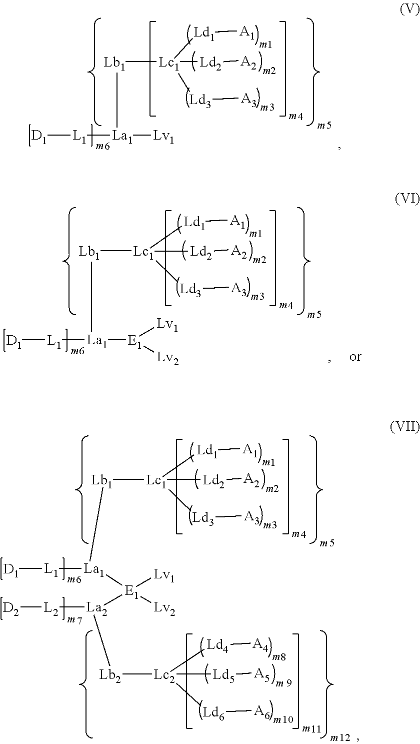
Or the conjugate of Formula (IV) is prepared through sequential conjugation reaction of Formula (V) and Formula (V′) to an antibody: 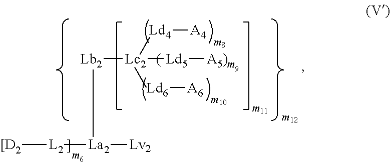
wherein D₁, D₂, L₁, L₂, La₁, La₂, Lb₁, Lb₂, Lc₁, Lc₂, Ld₁, Ld₂, Ld₃, Ld₄, Ld₅, Ld₆, A₁, A₂, A₃. A₄, A₅, A₆, Ei, m₁, m₂, m₃, m₄, m₅, m₆, m₇, m₈, m₉, m₁₀, m₁₁, and m₁₂ are defined the same in -claim 1; Lv₁ and Lv₂ are a reactive group and are independently or fused jointly selected from: 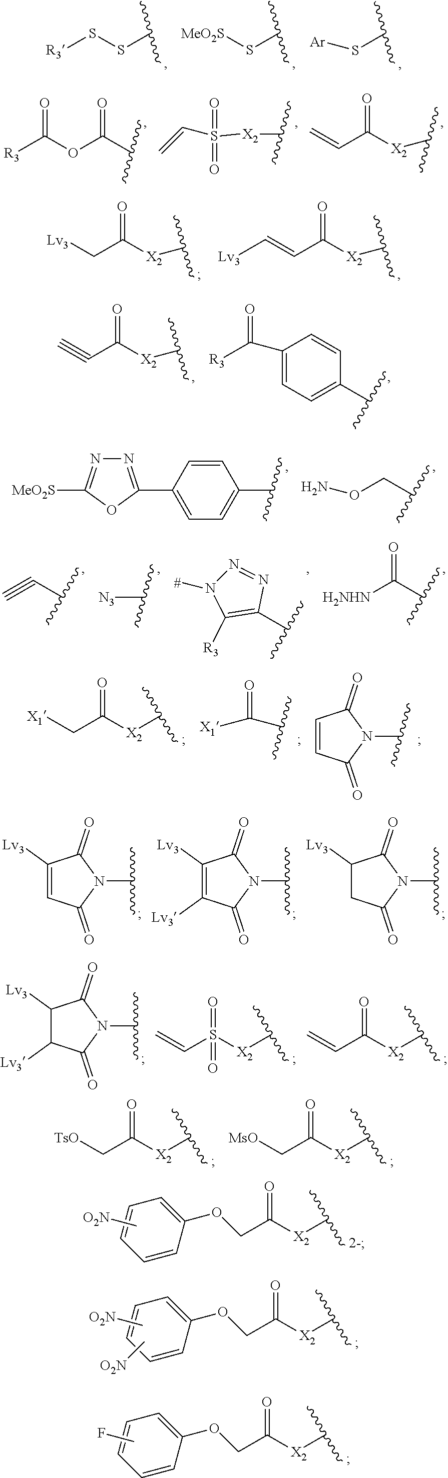 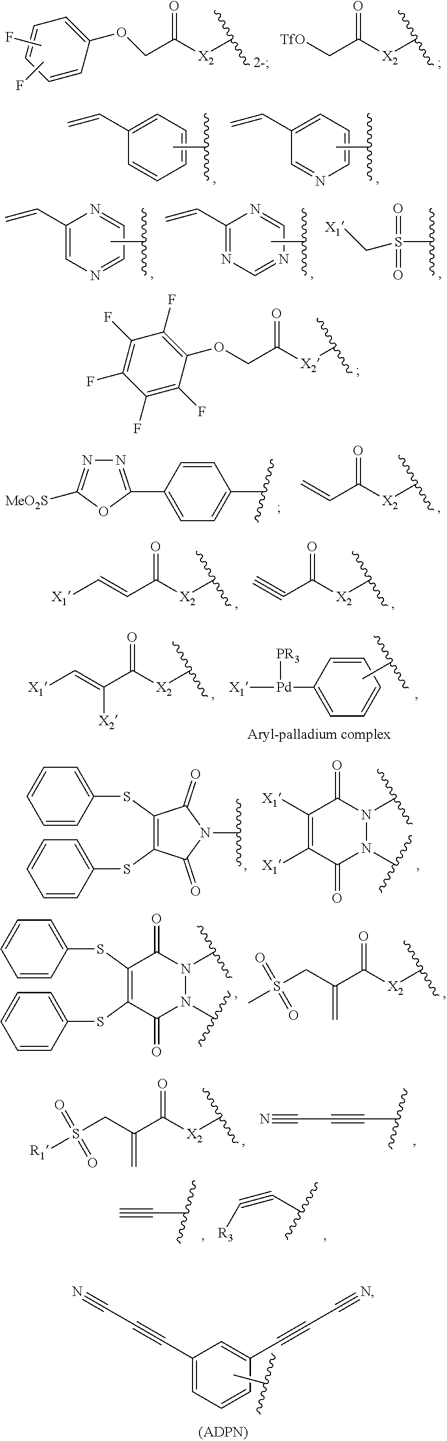 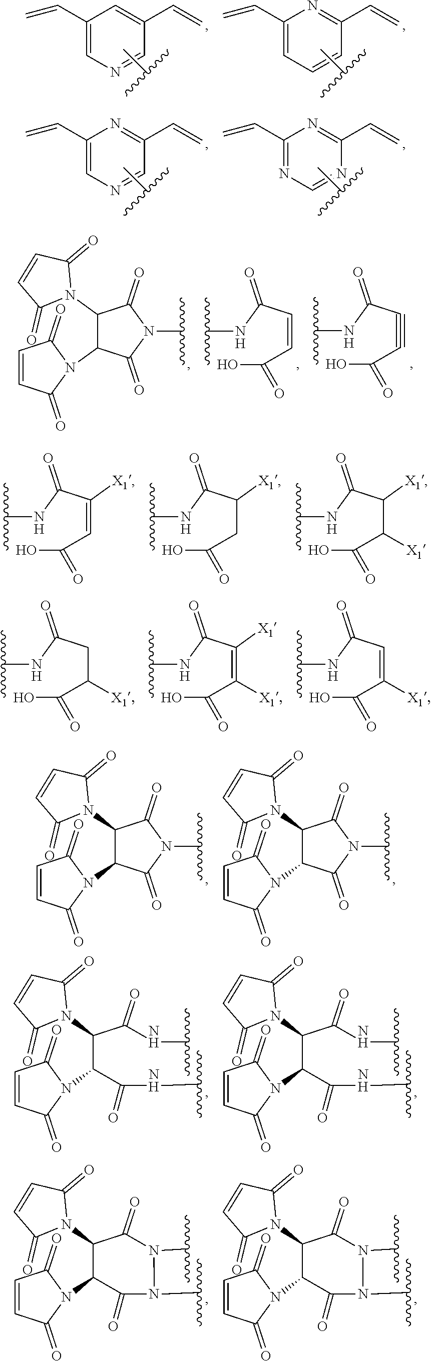 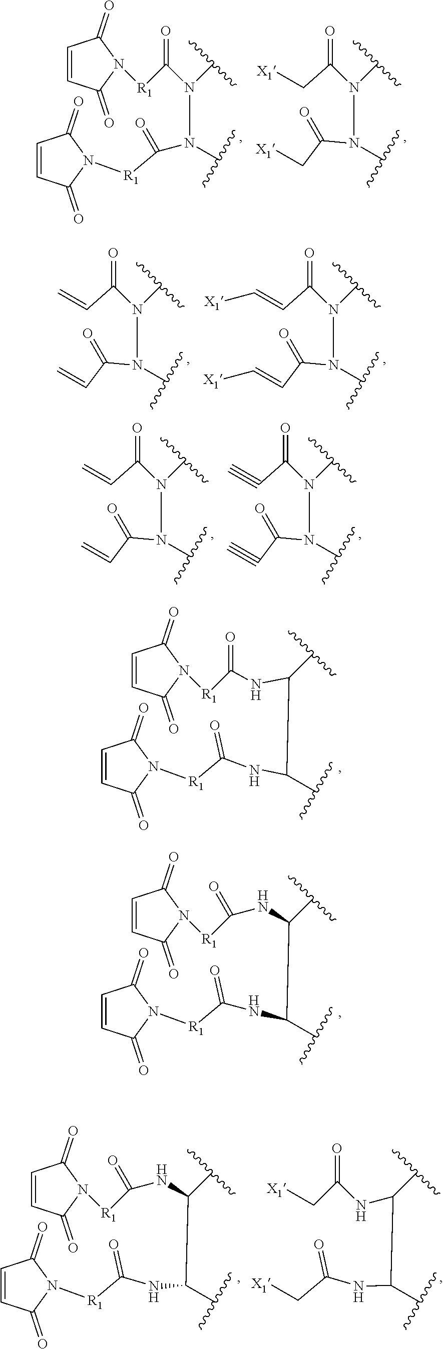 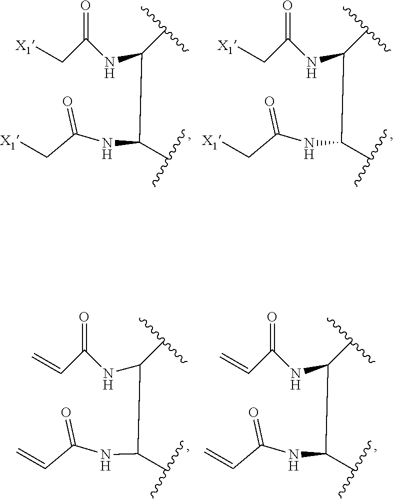 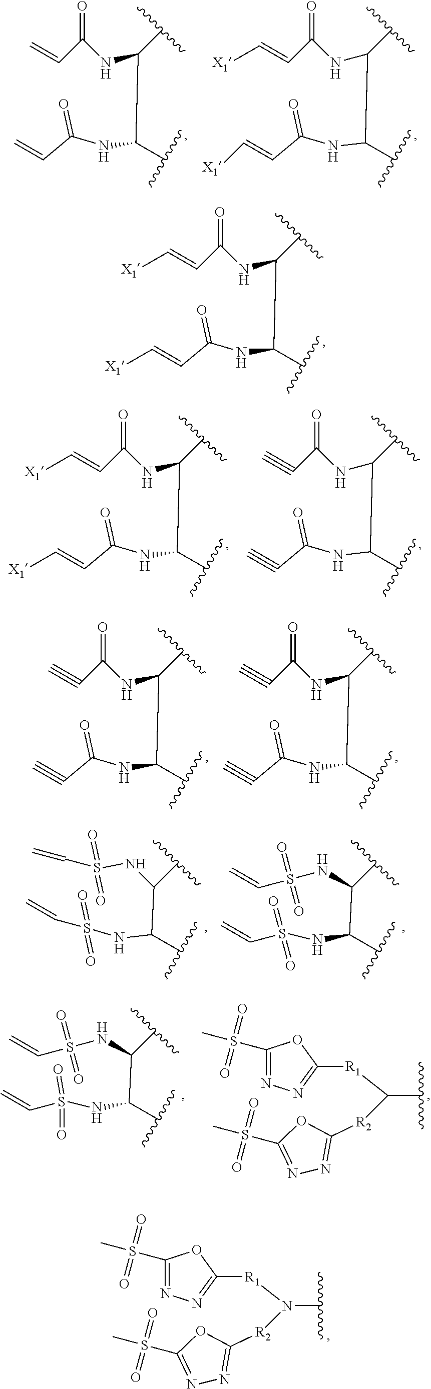 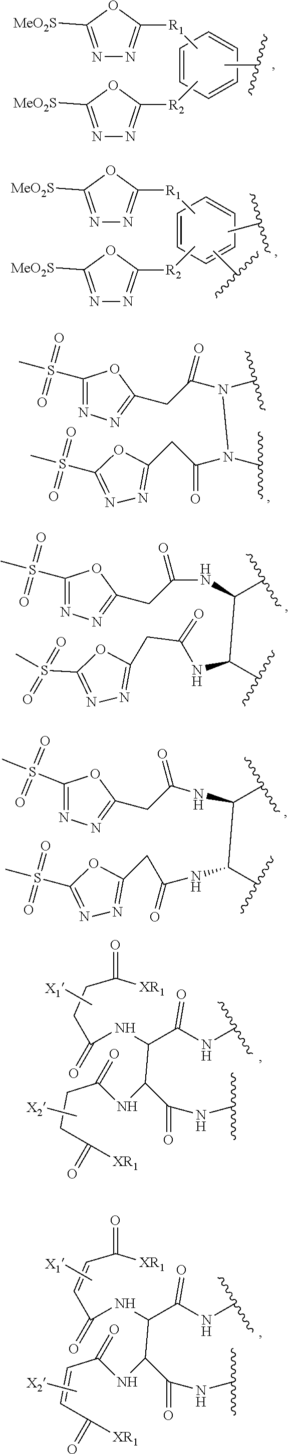 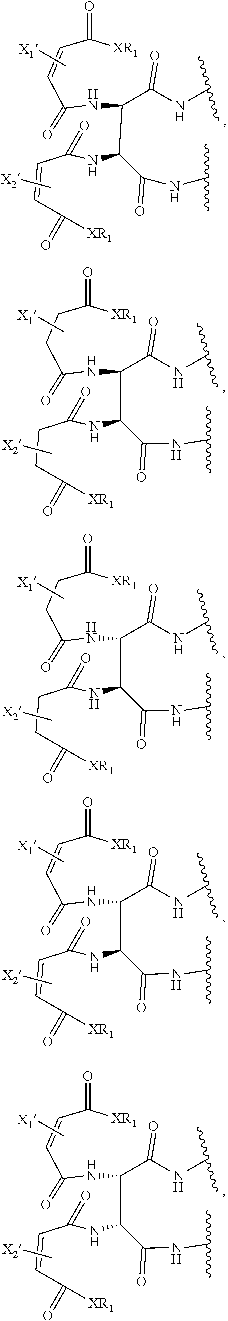 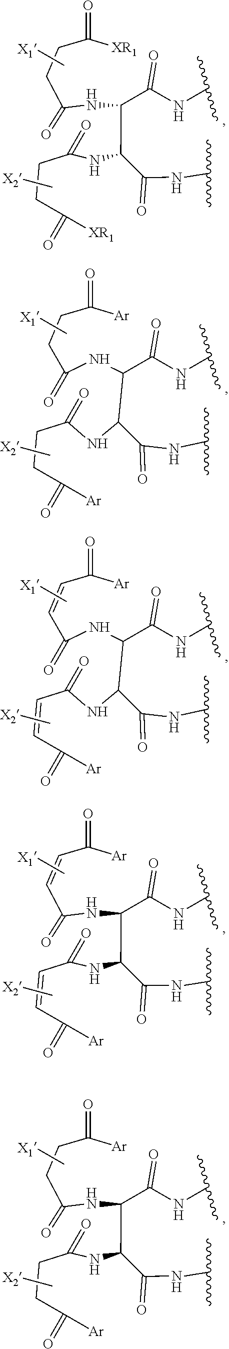 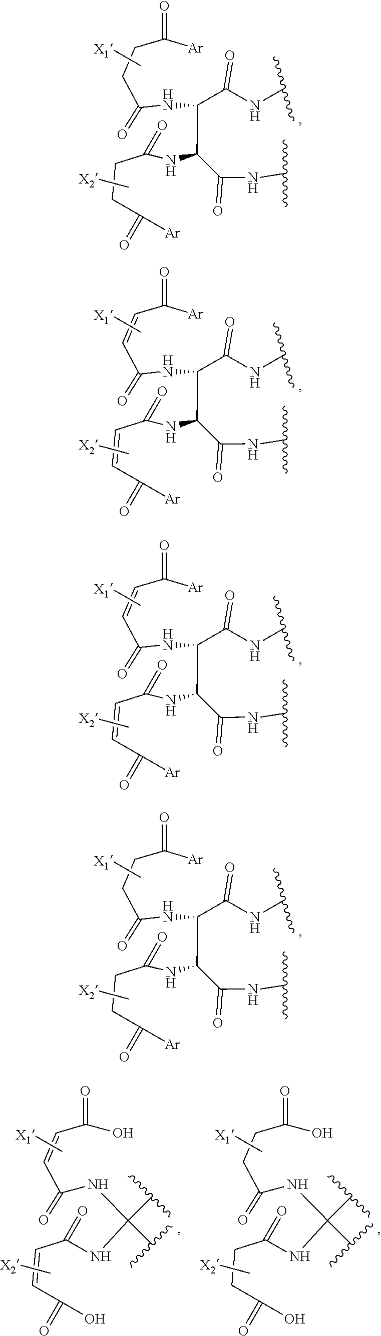 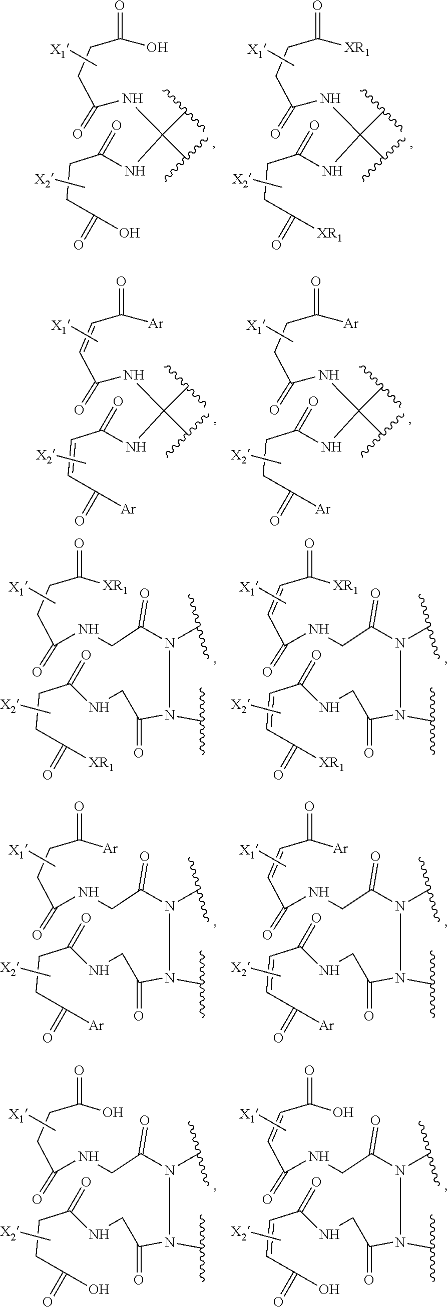 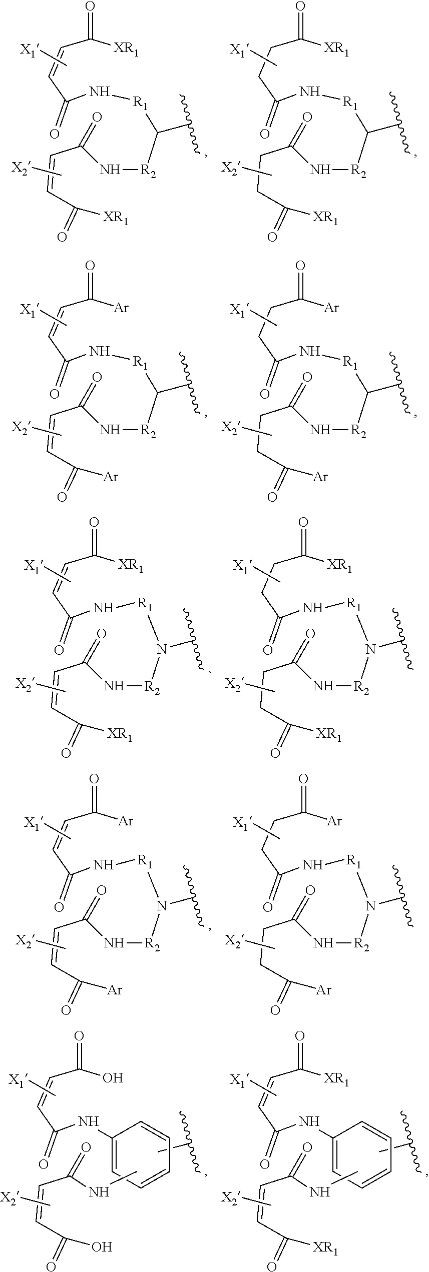 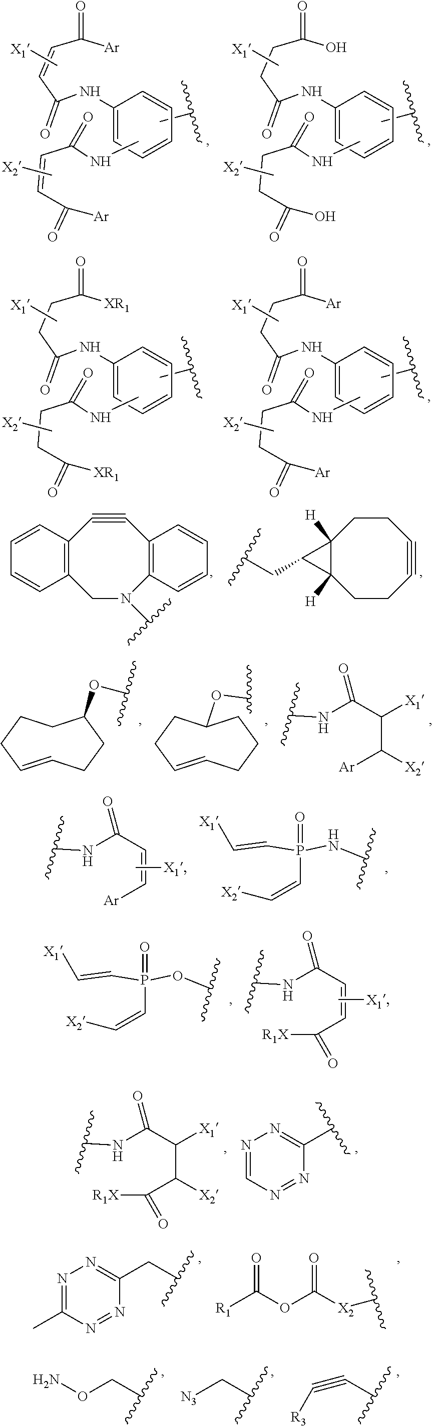 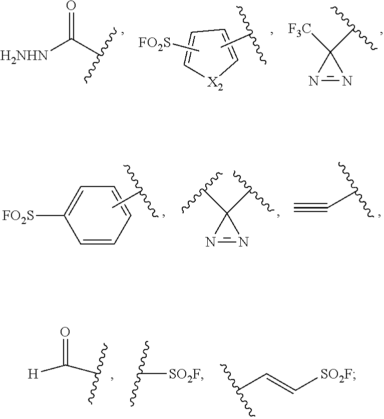
wherein X₁′ and X₂′ are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, —halogen, —OR₁, —SR₁, —NR¹R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

11. The antibody drug conjugate according to claim 10, wherein in Formula (VI) and (VII), the joint structure of 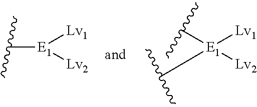
are accordingly selected from: 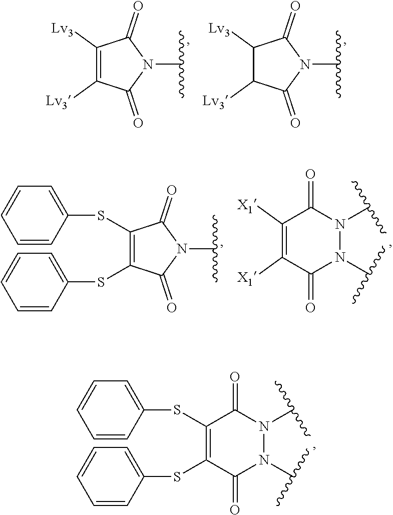 [chemical structure] 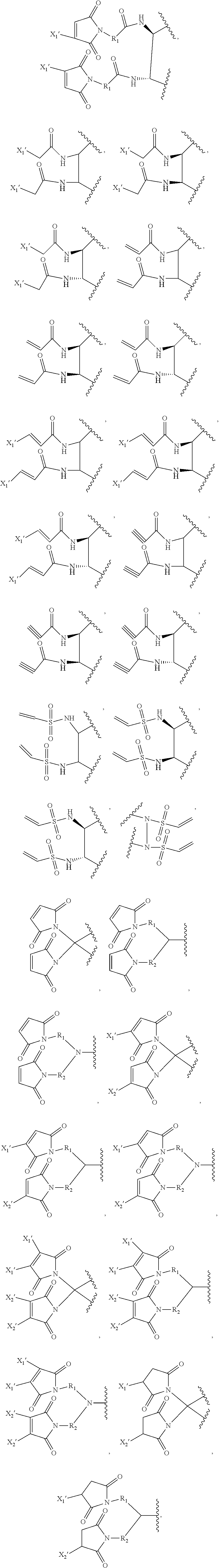 [chemical structure] [chemical structure] 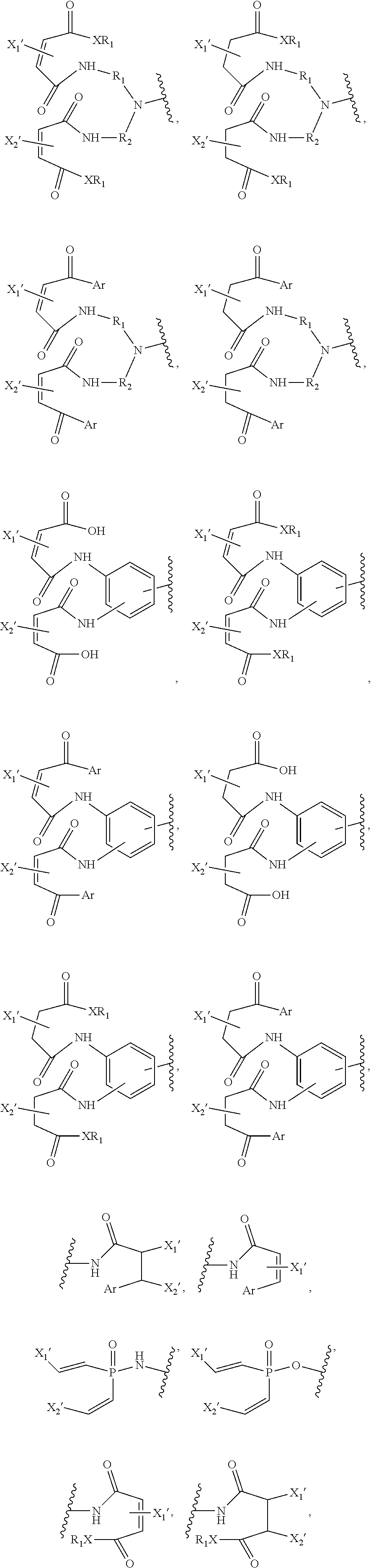
wherein Lv₃, Lv₃′, X₁′ and X₂′are described above in claim 10; the connecting bond “—” in the middle of the two atoms means it can link either one of the two atoms.

12. The antibody drug conjugate according to claim 10, wherein the compound is represented as the following structures: 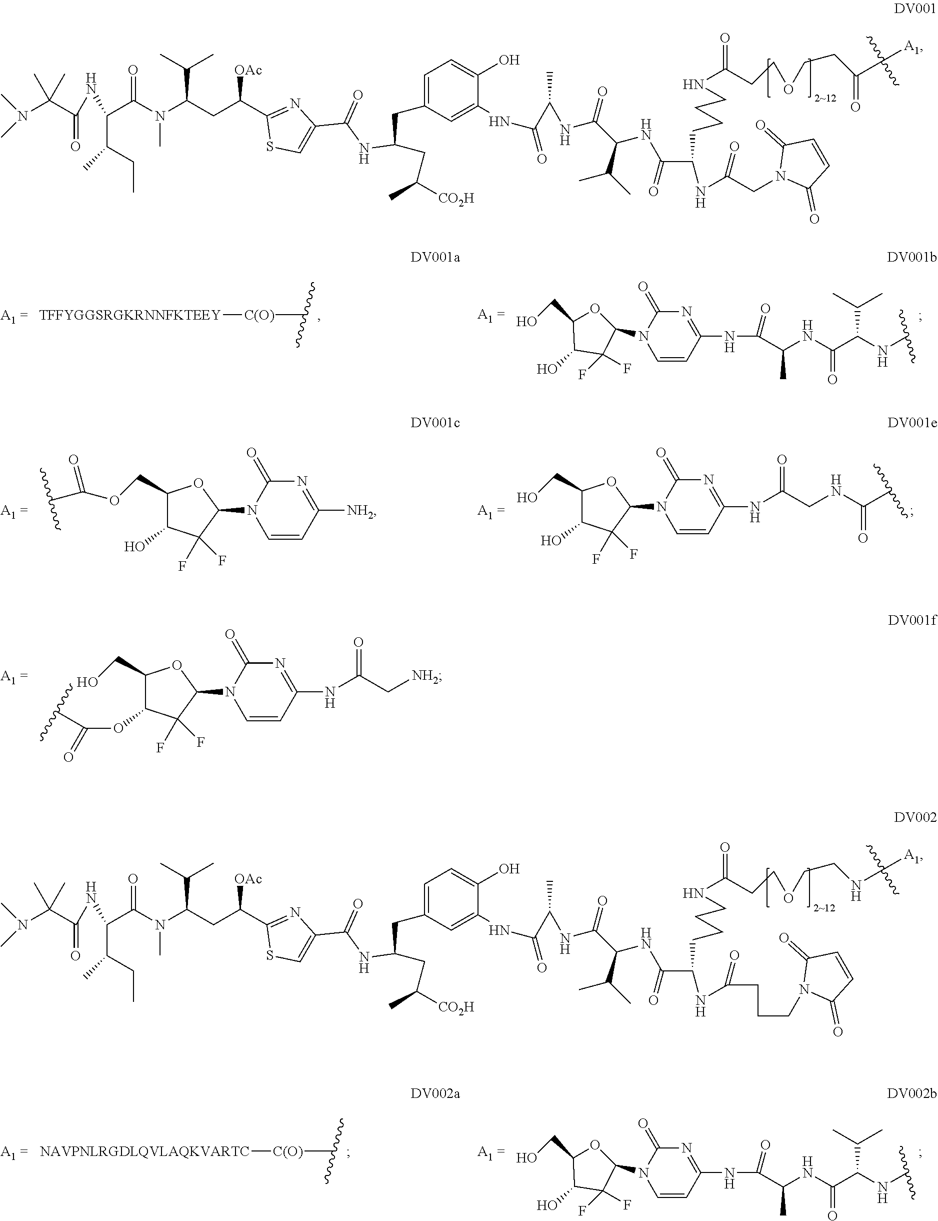 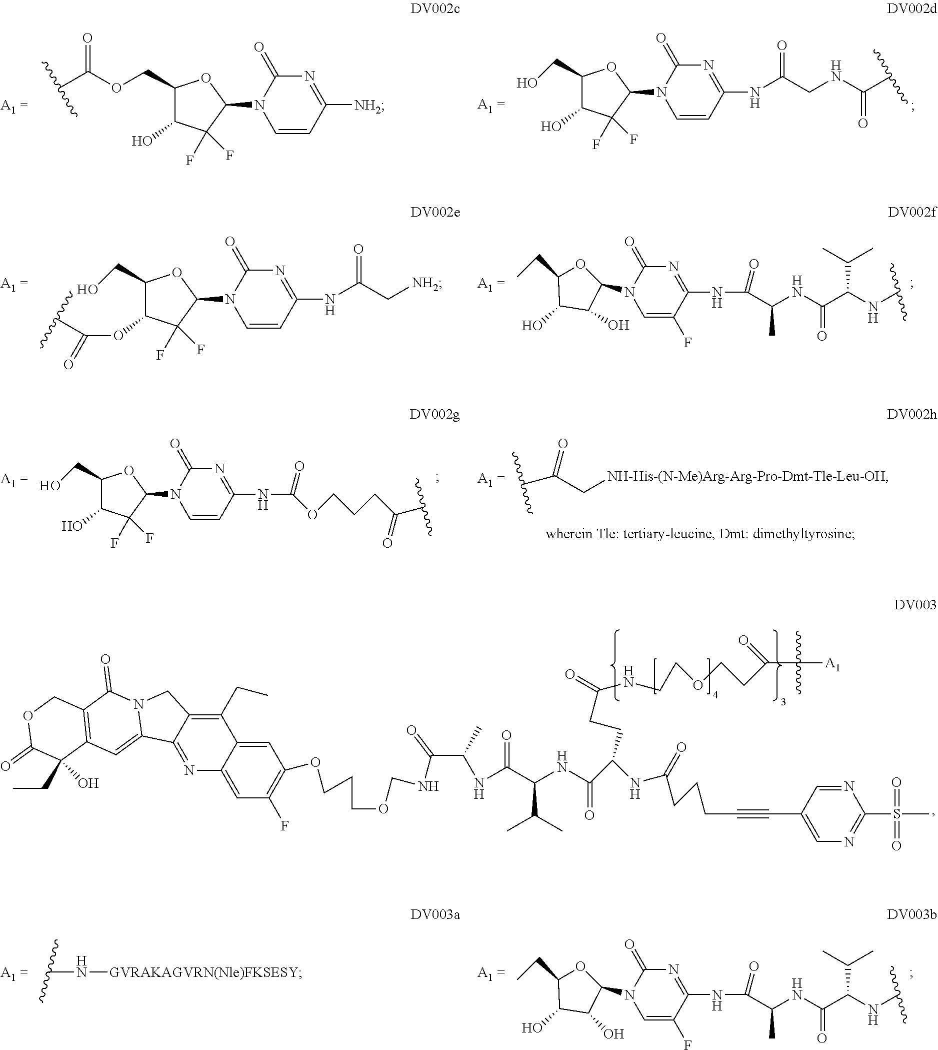 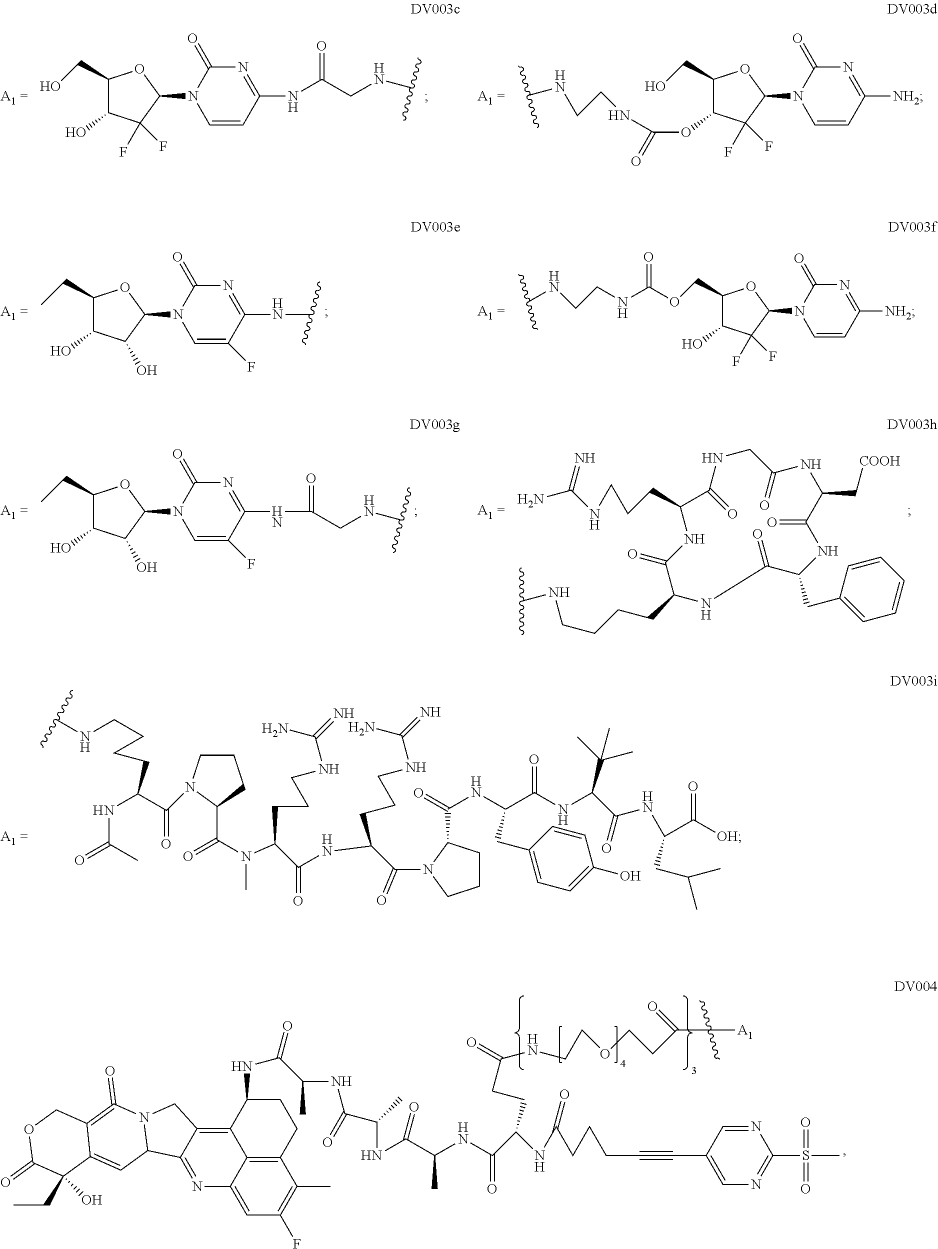 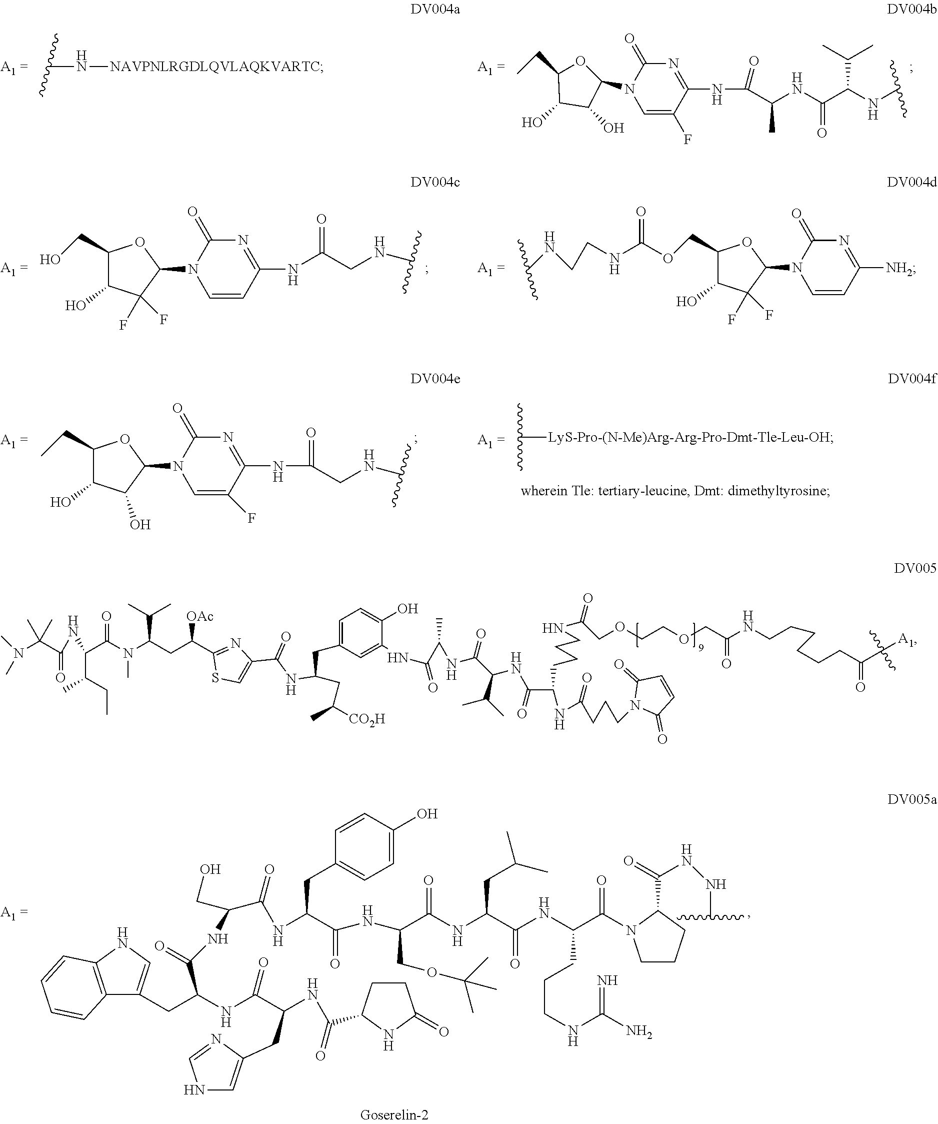 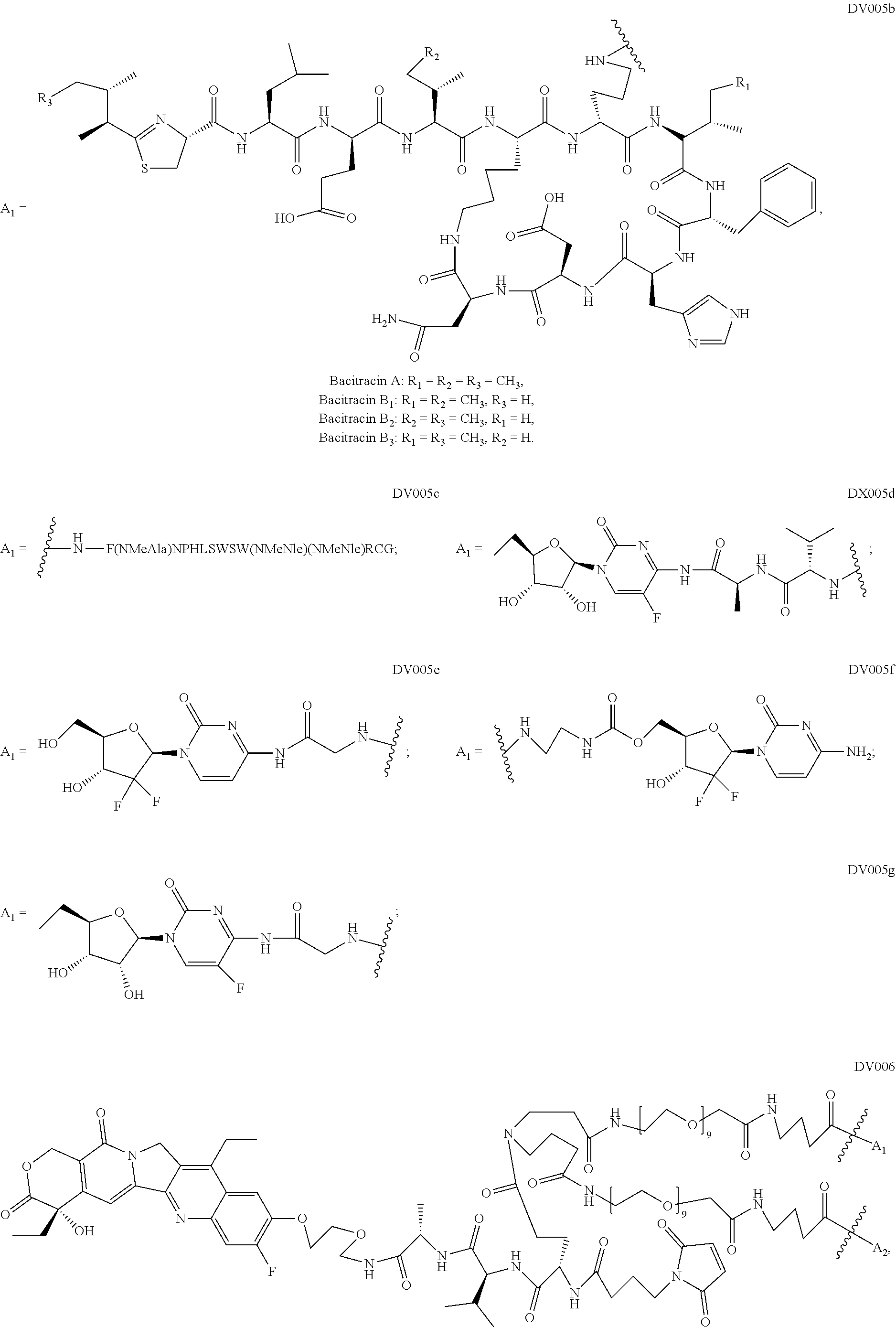 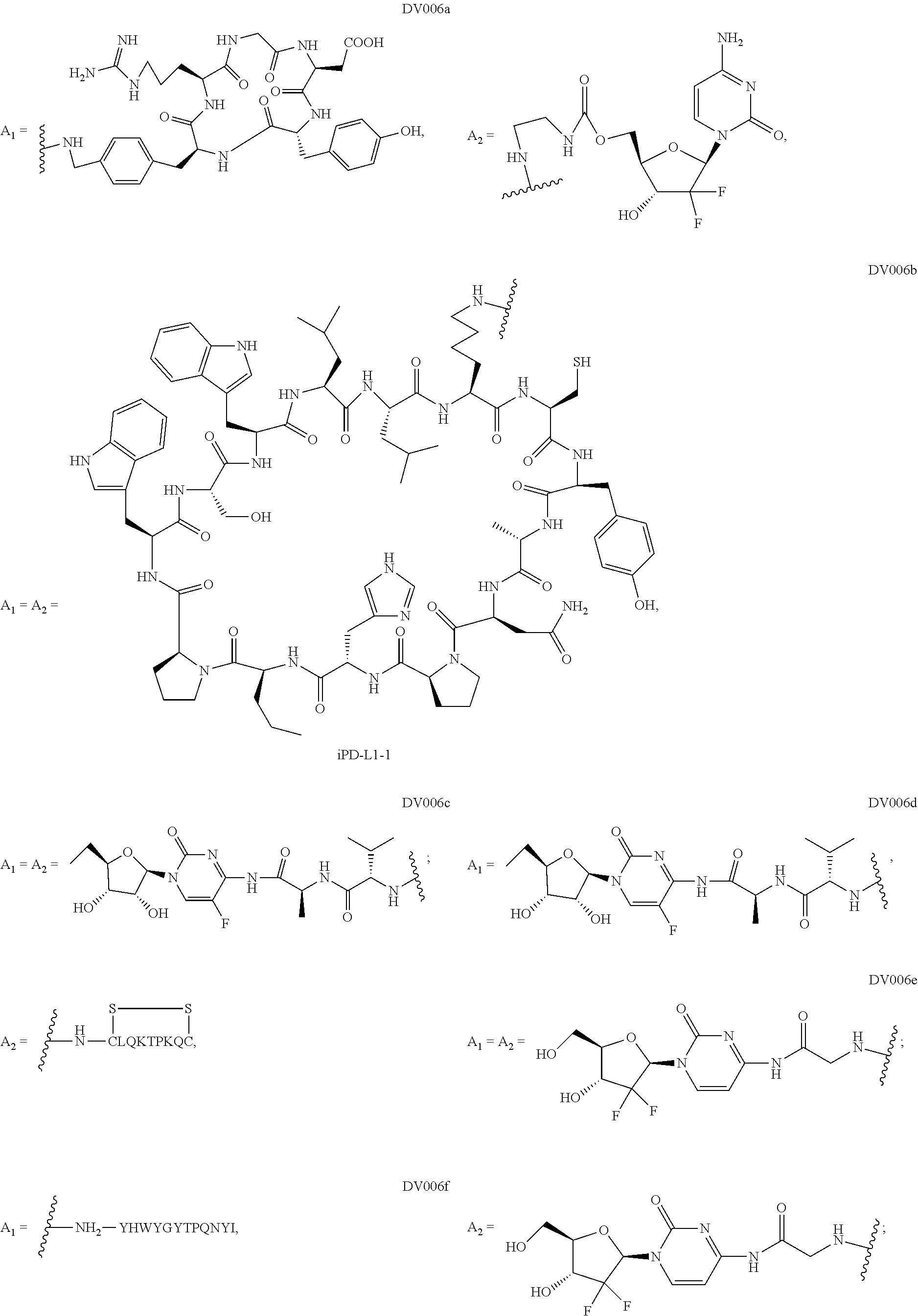 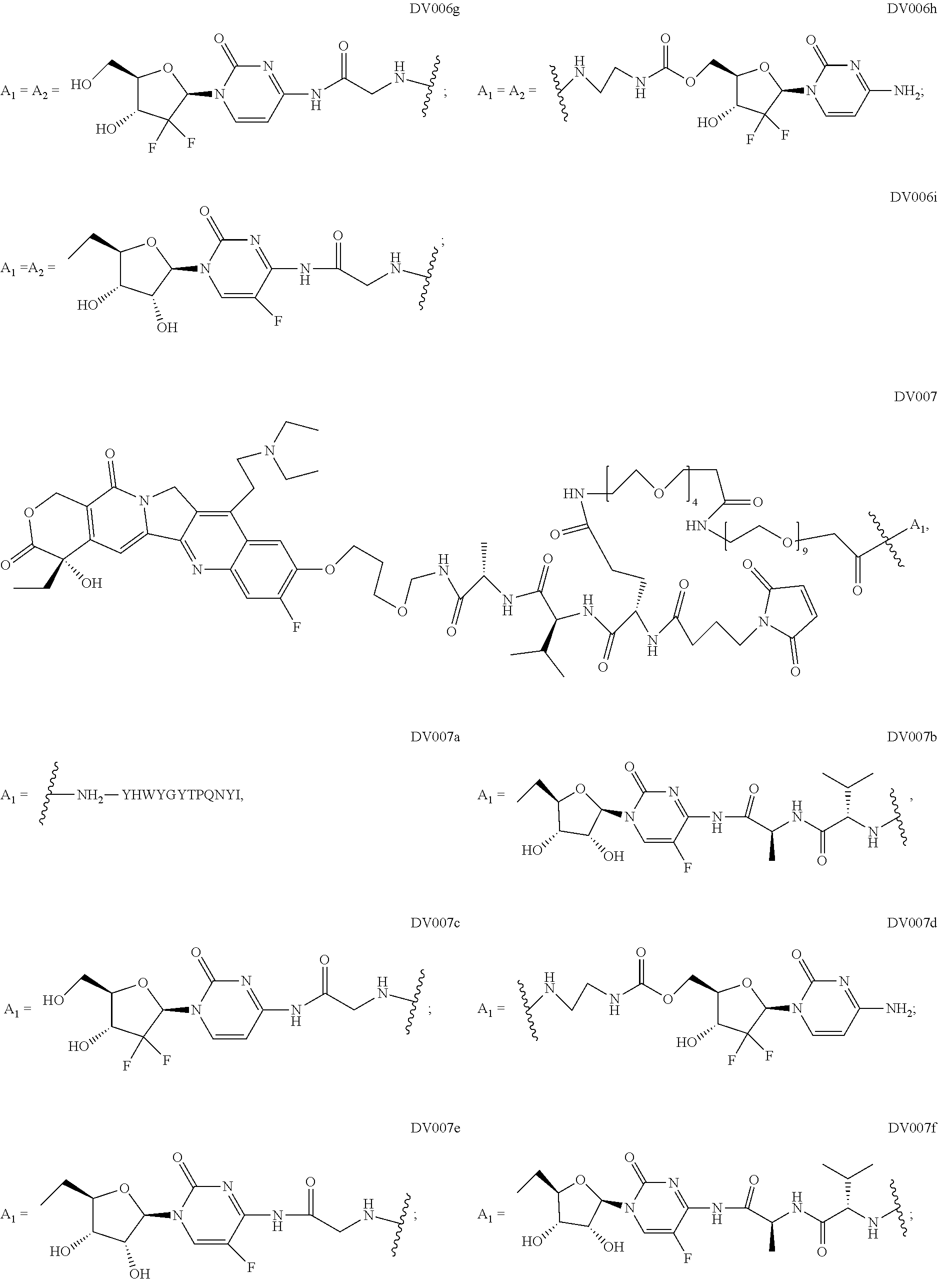 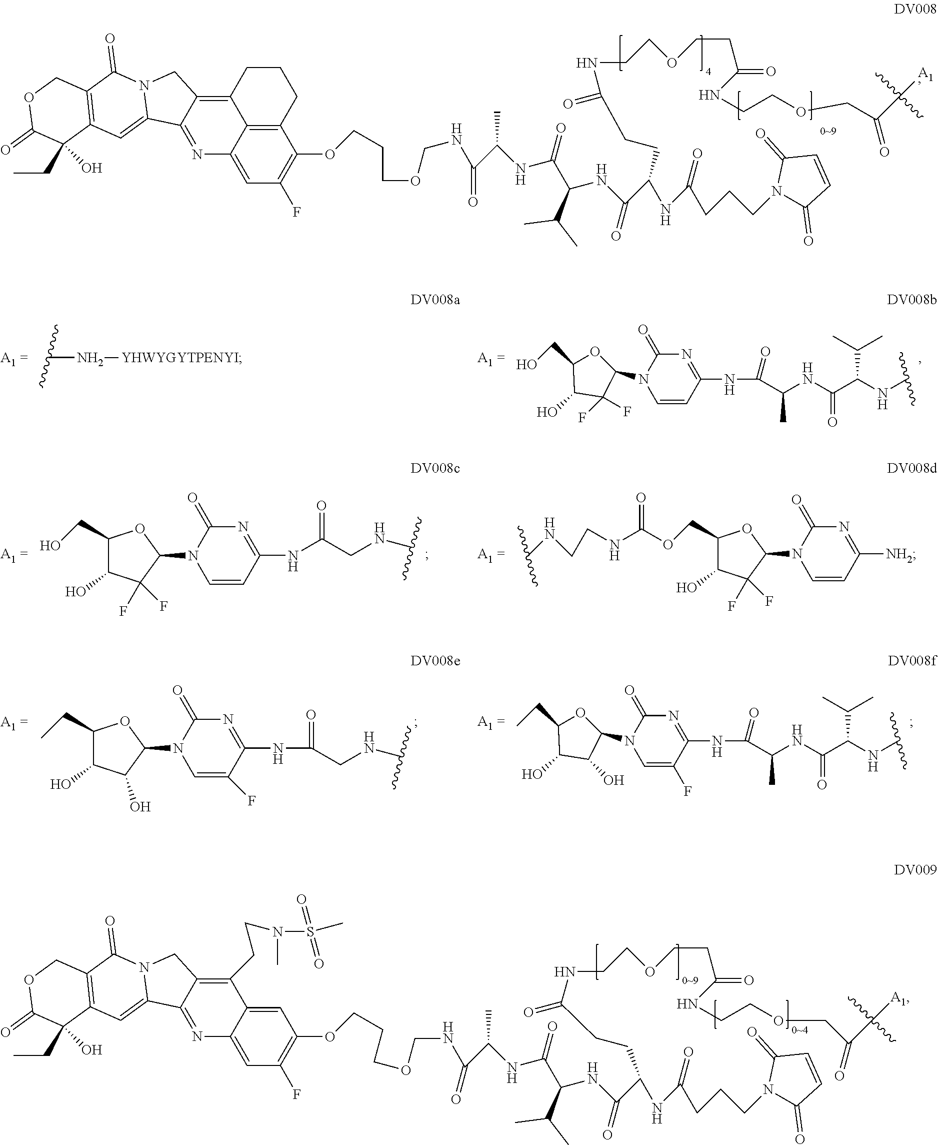 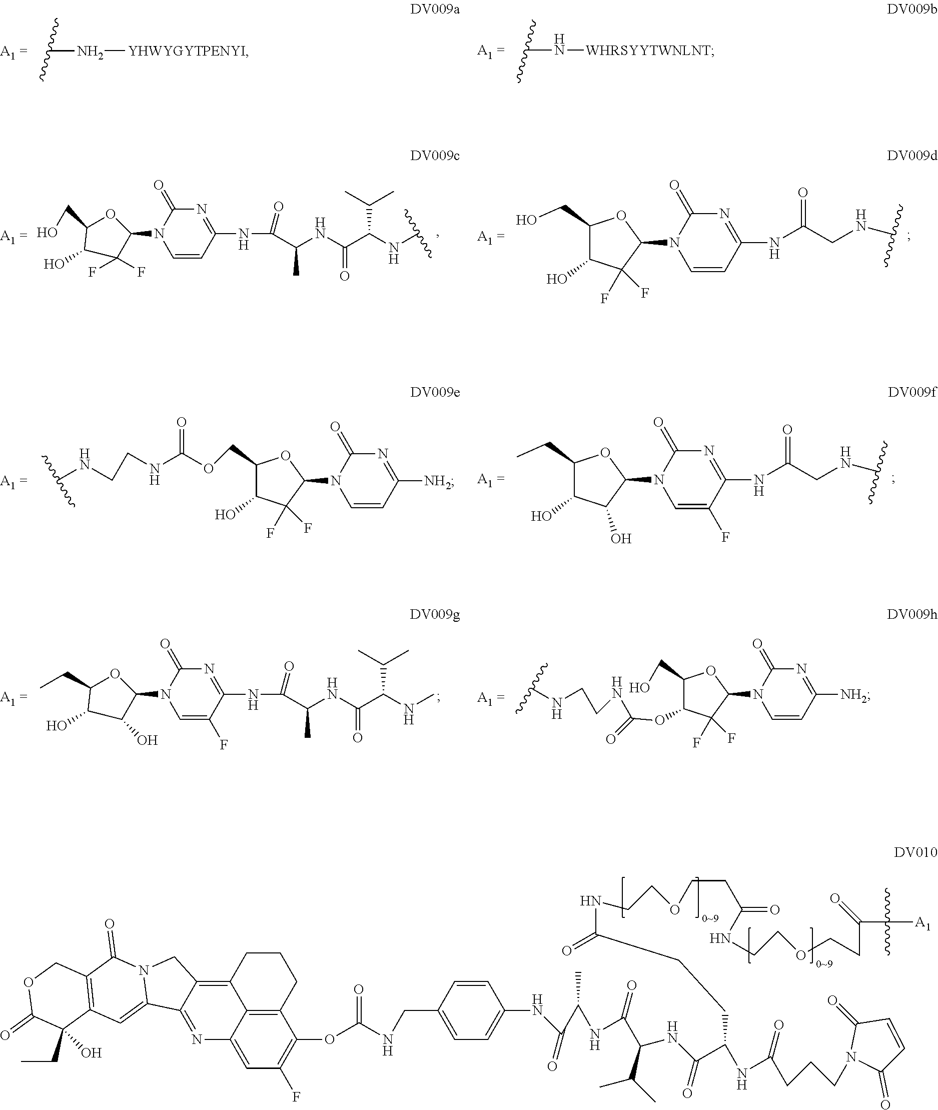 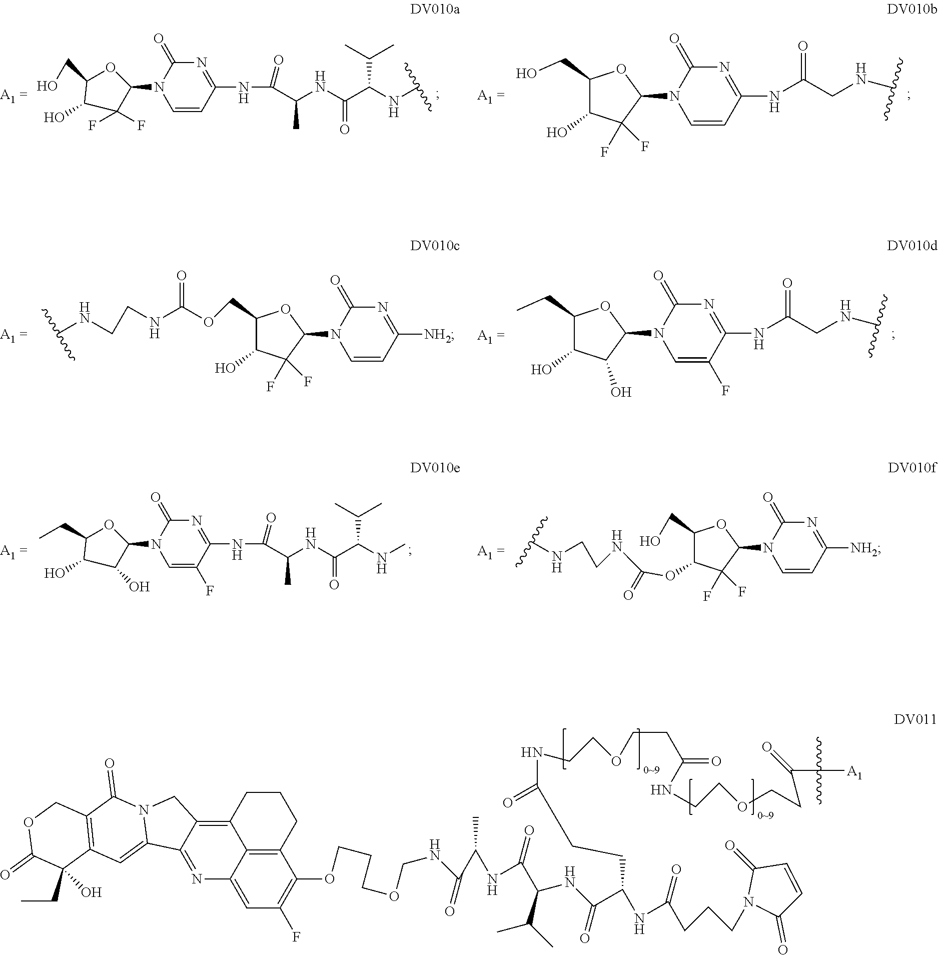 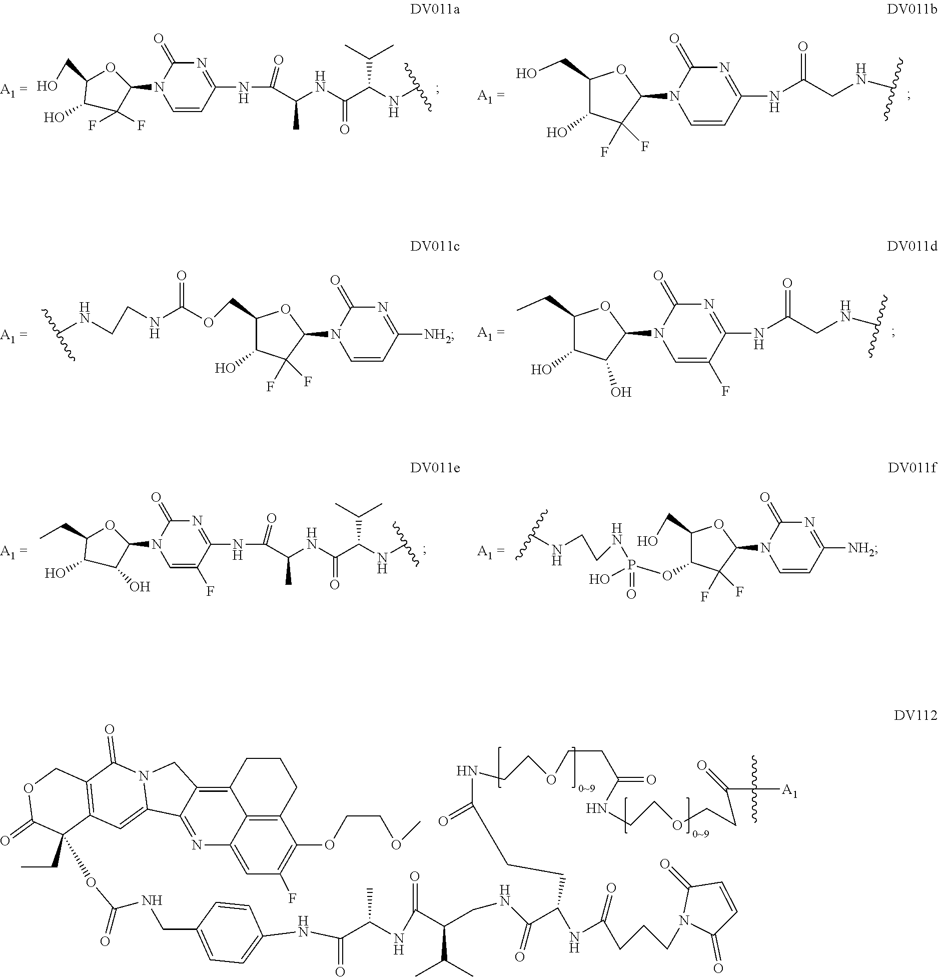 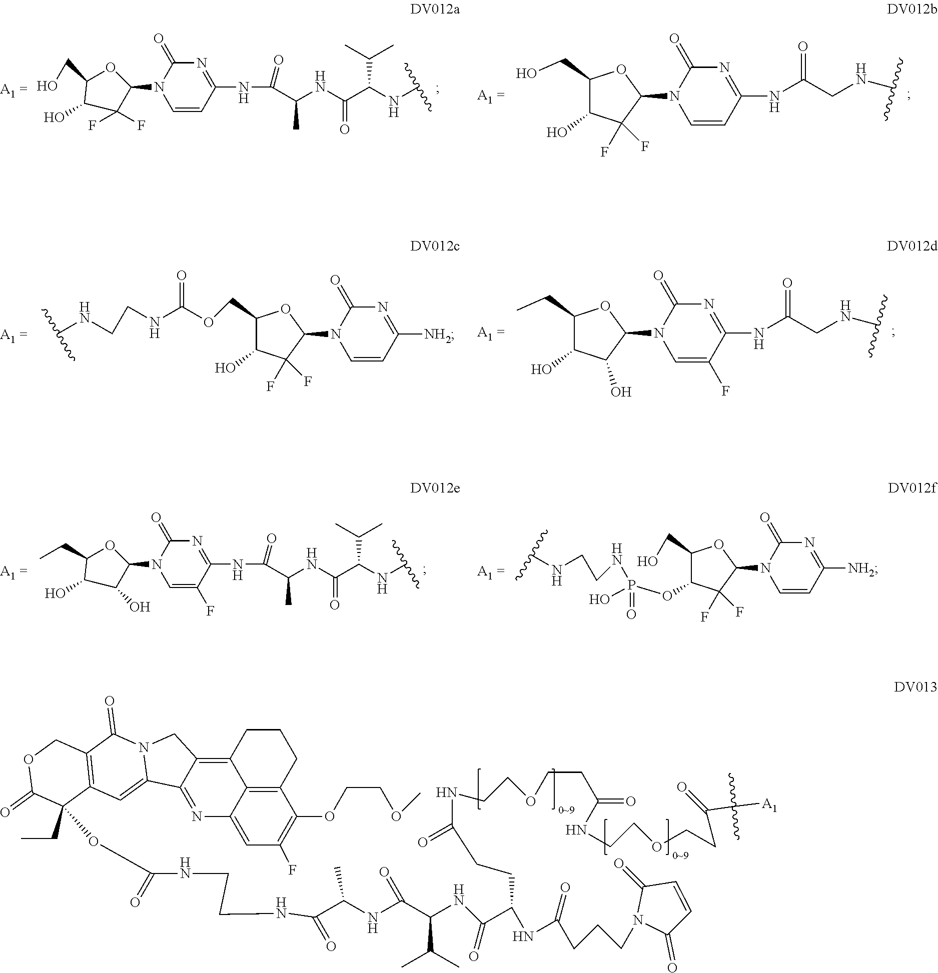 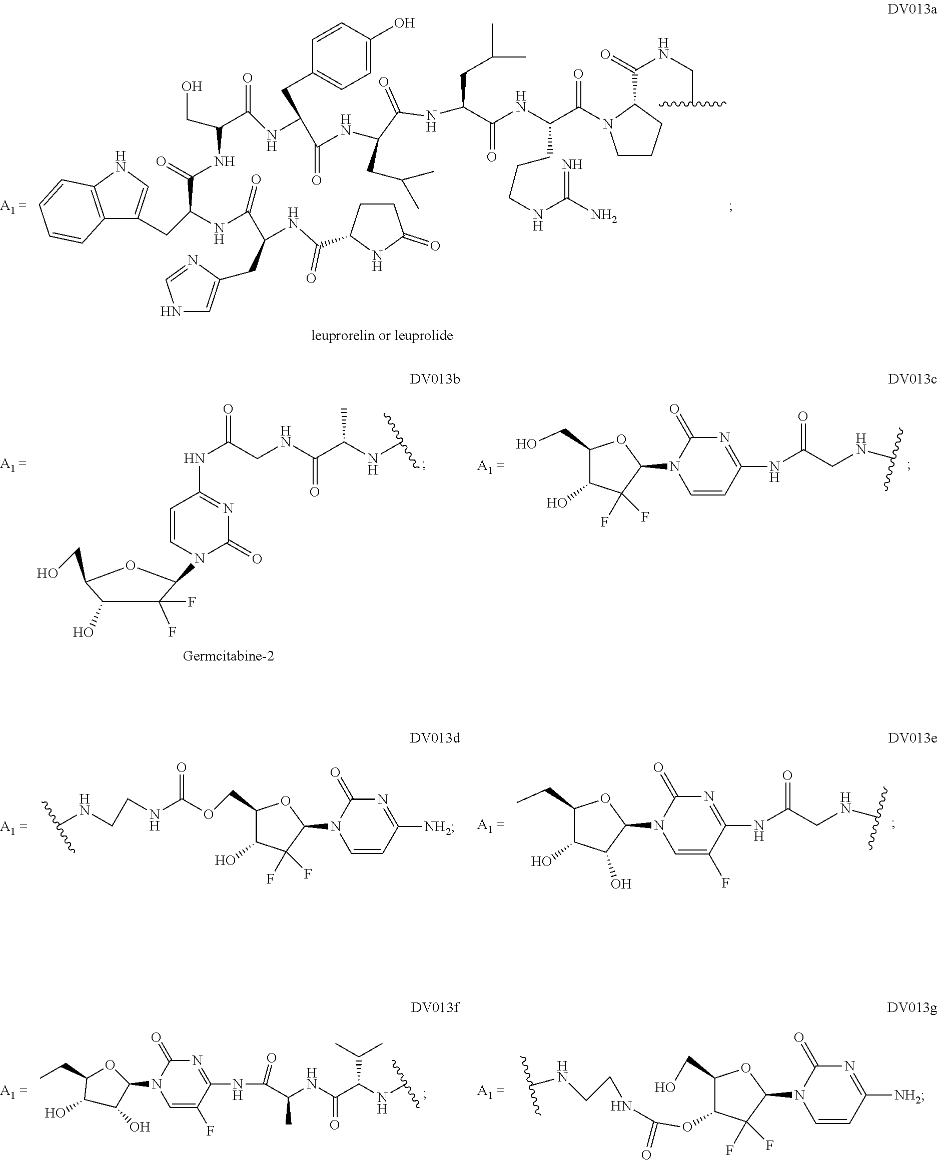 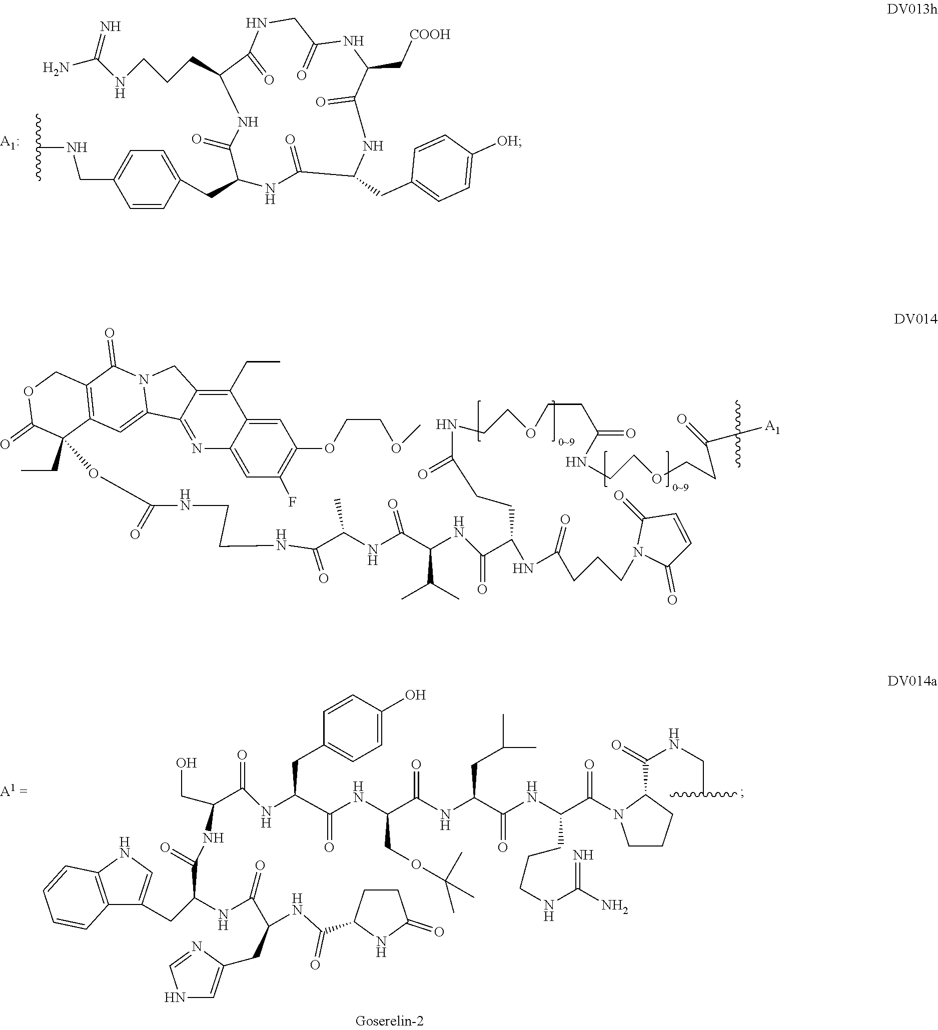 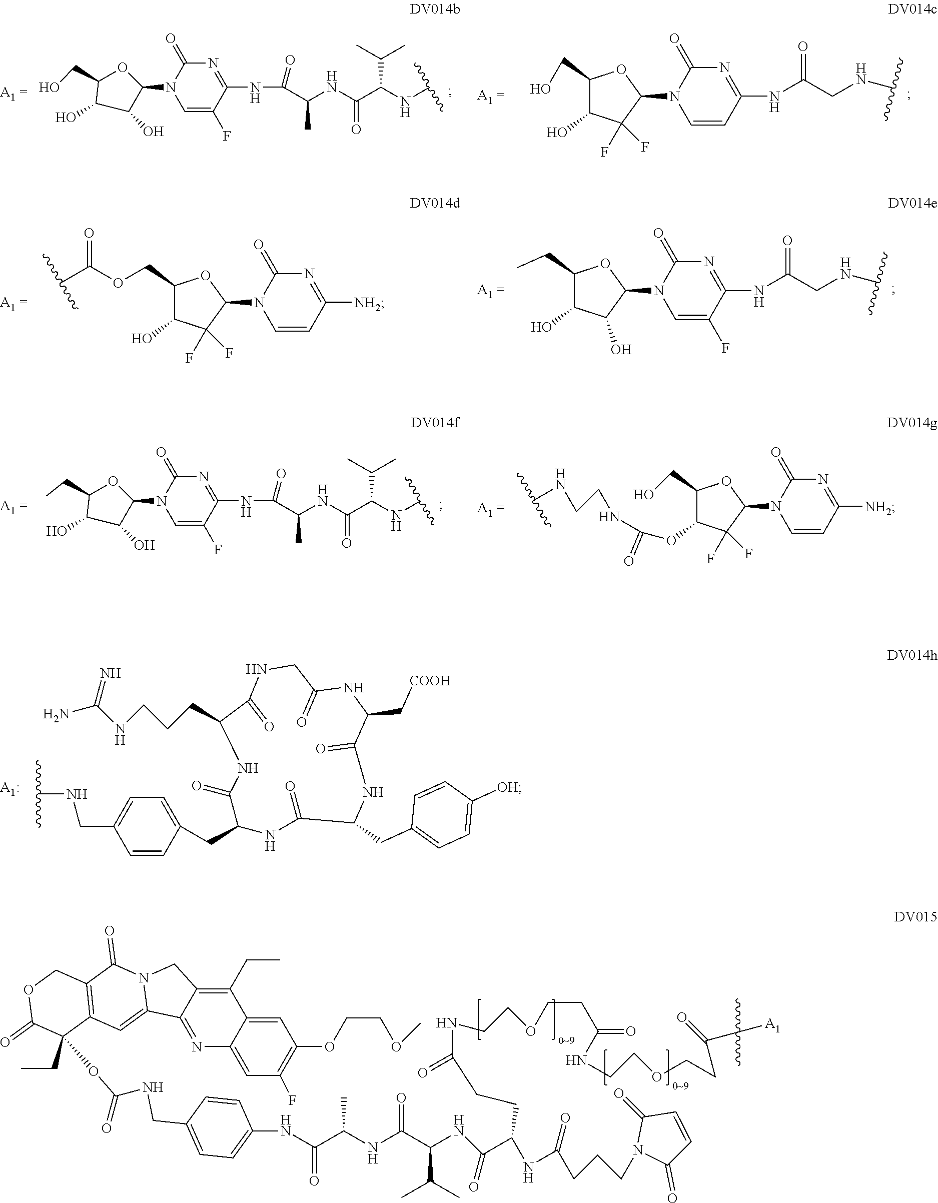 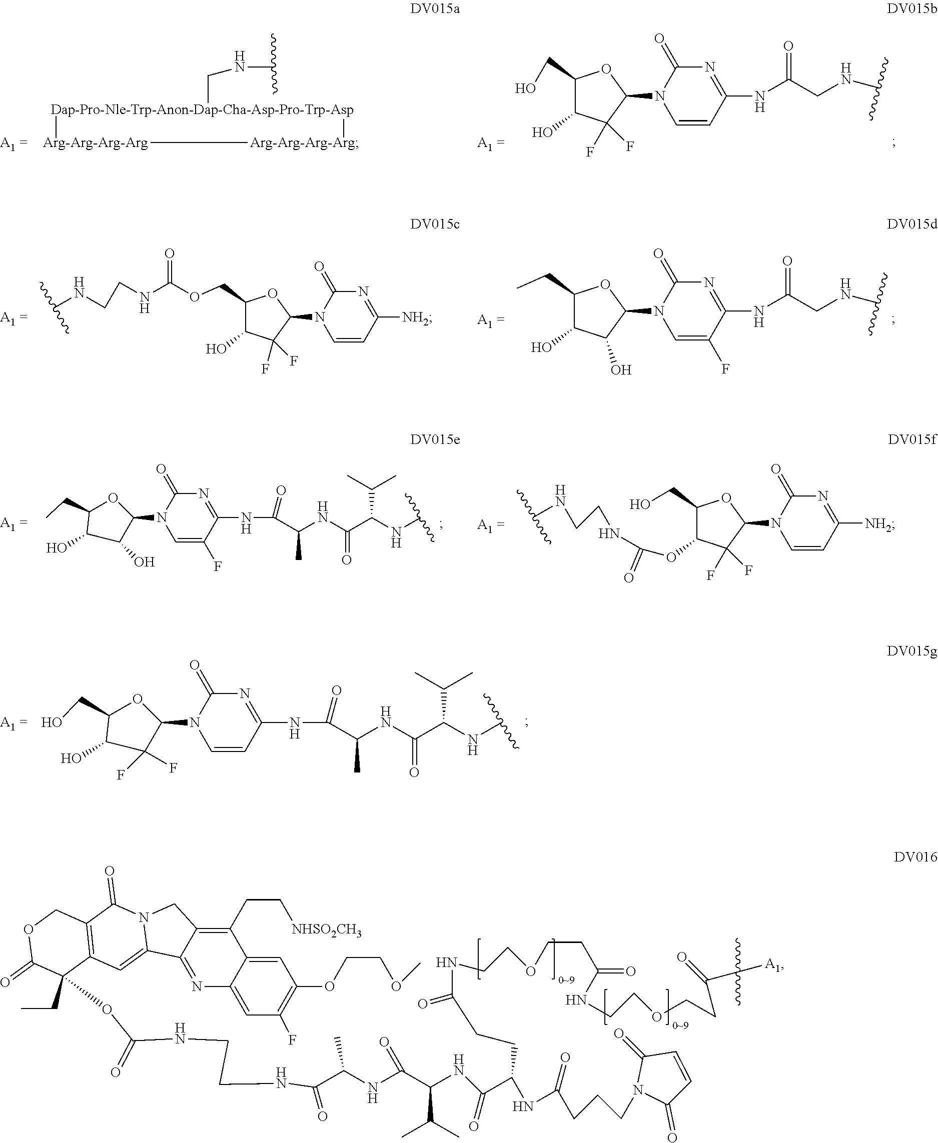 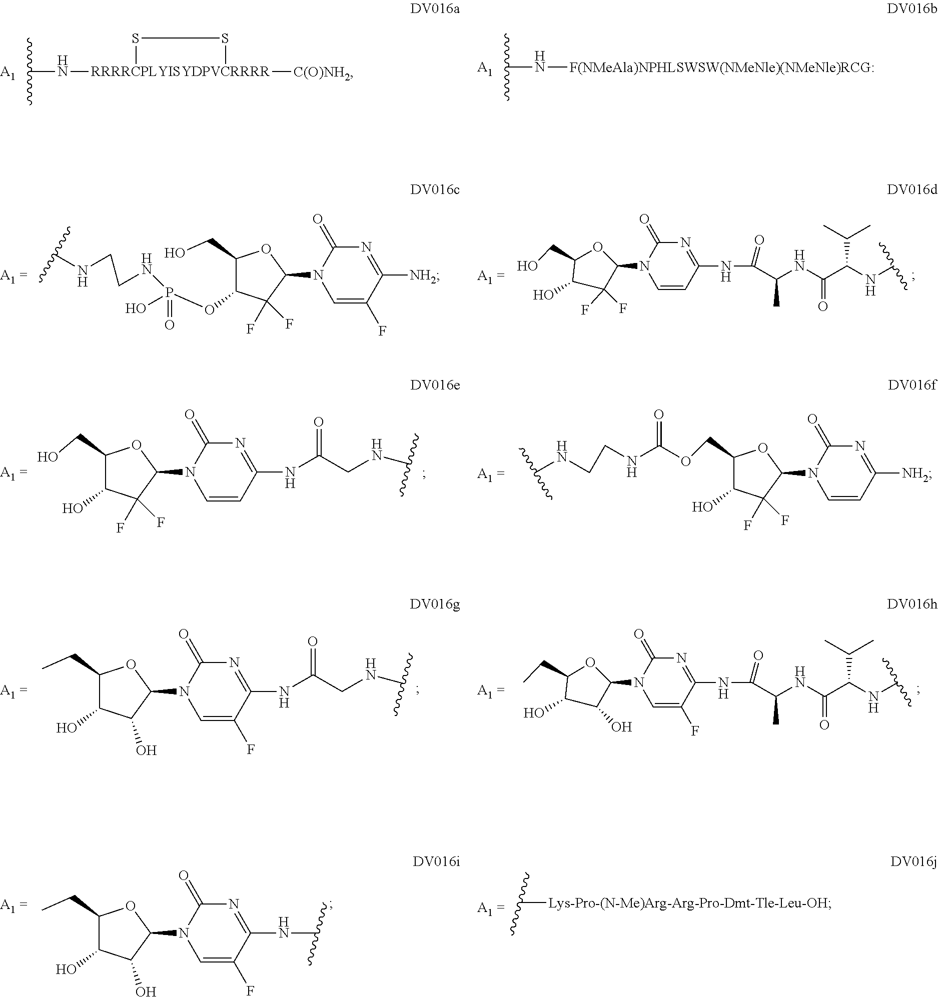 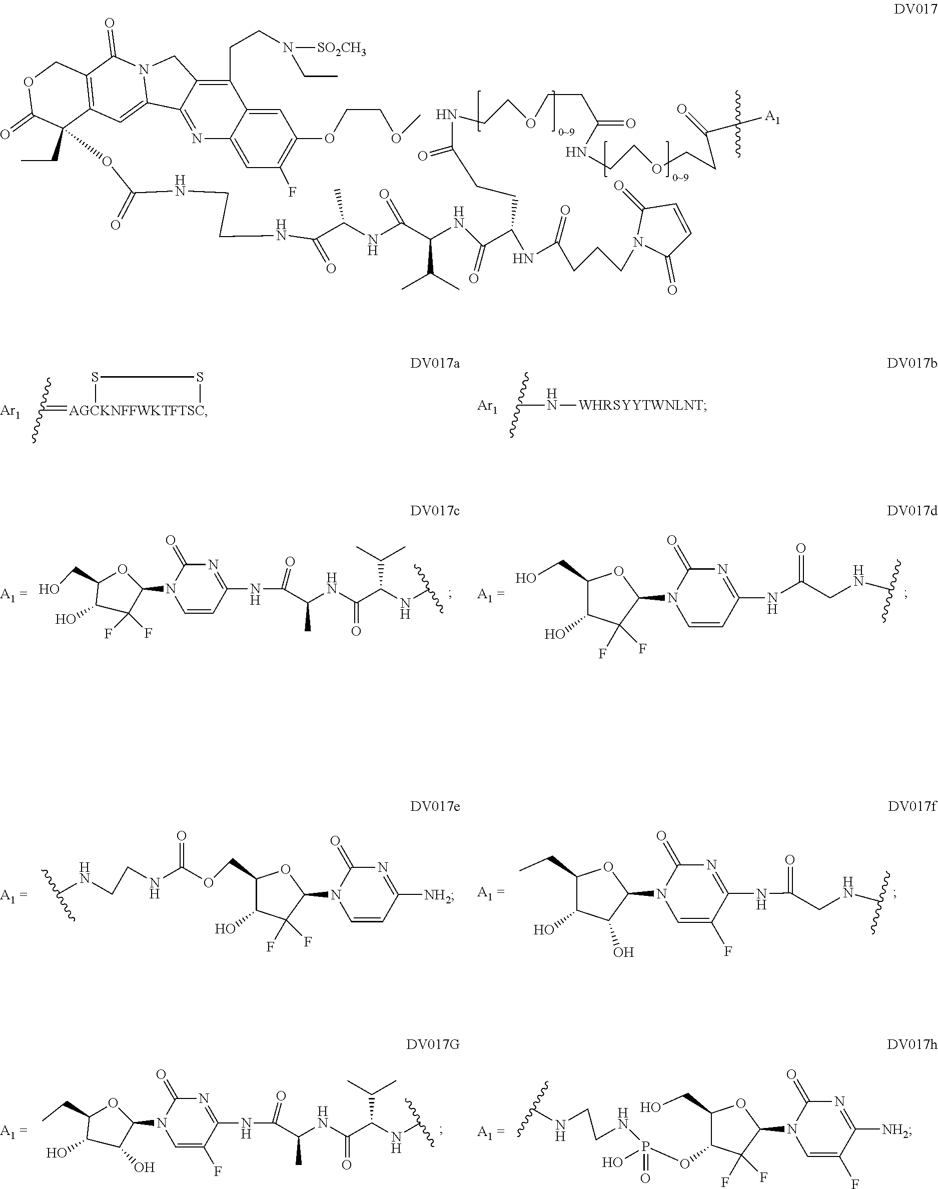 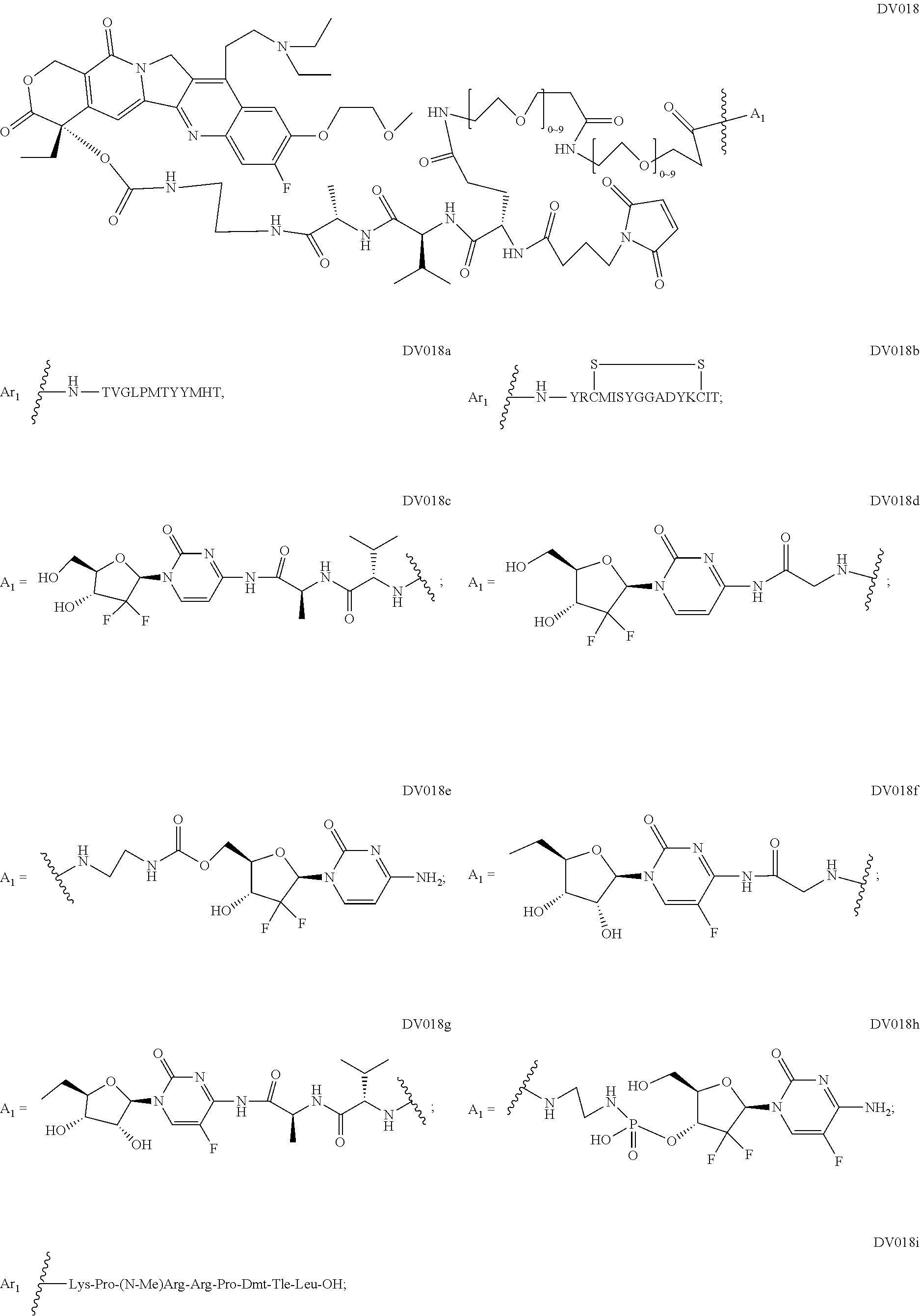 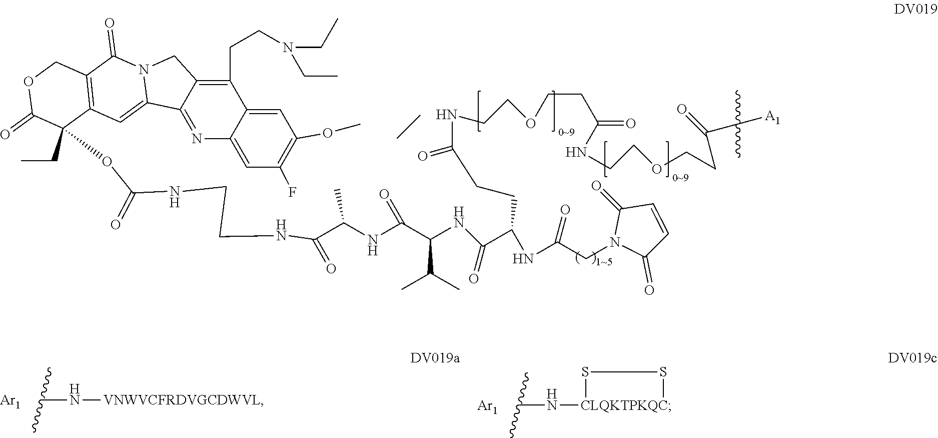 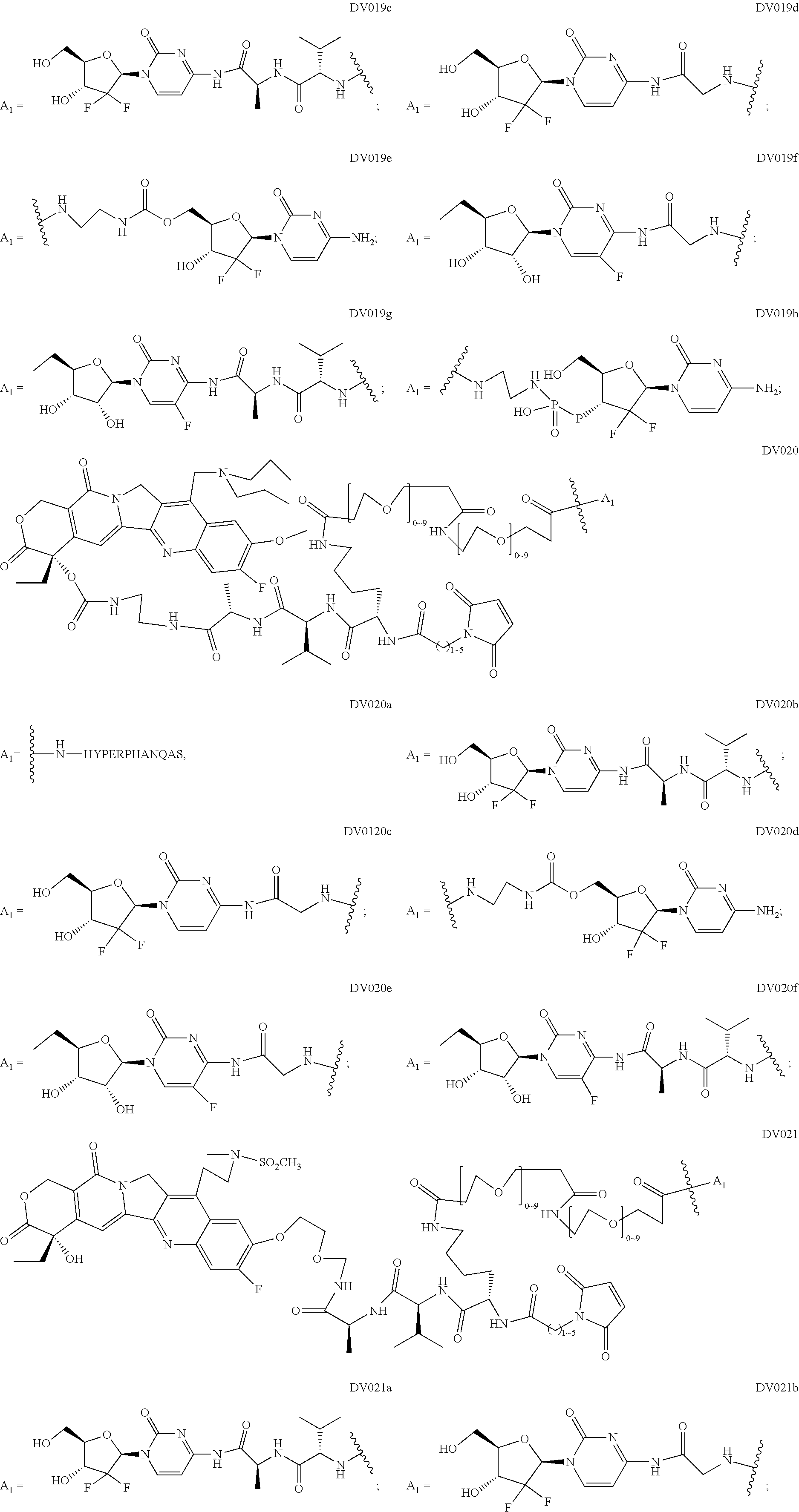 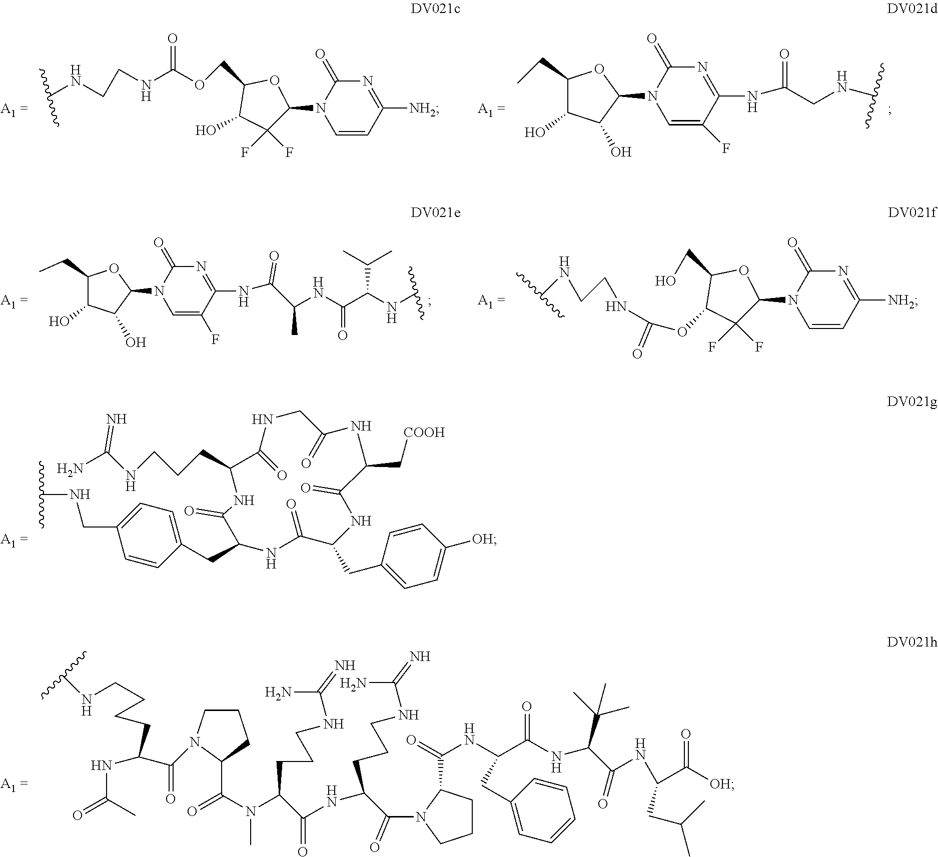 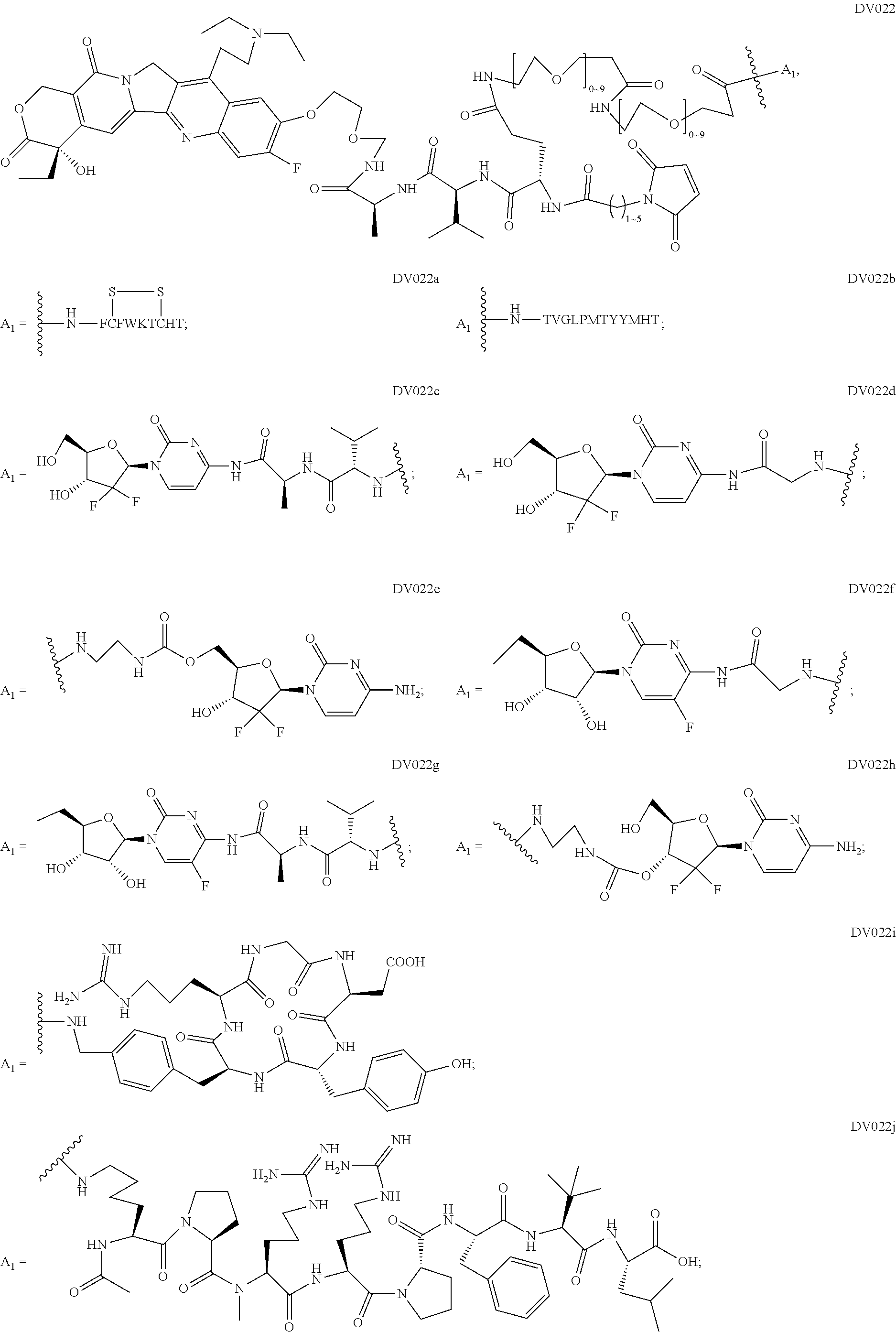 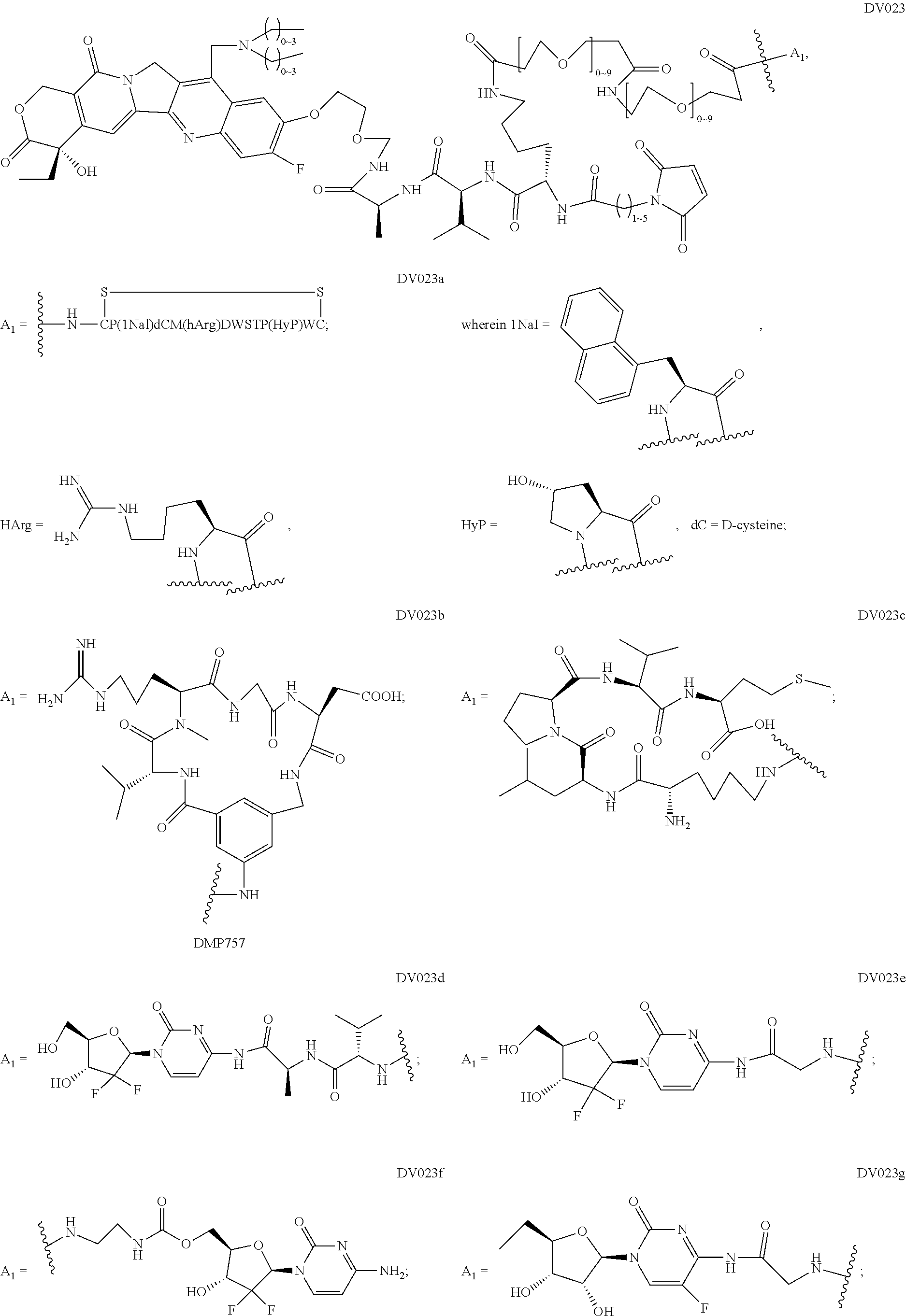 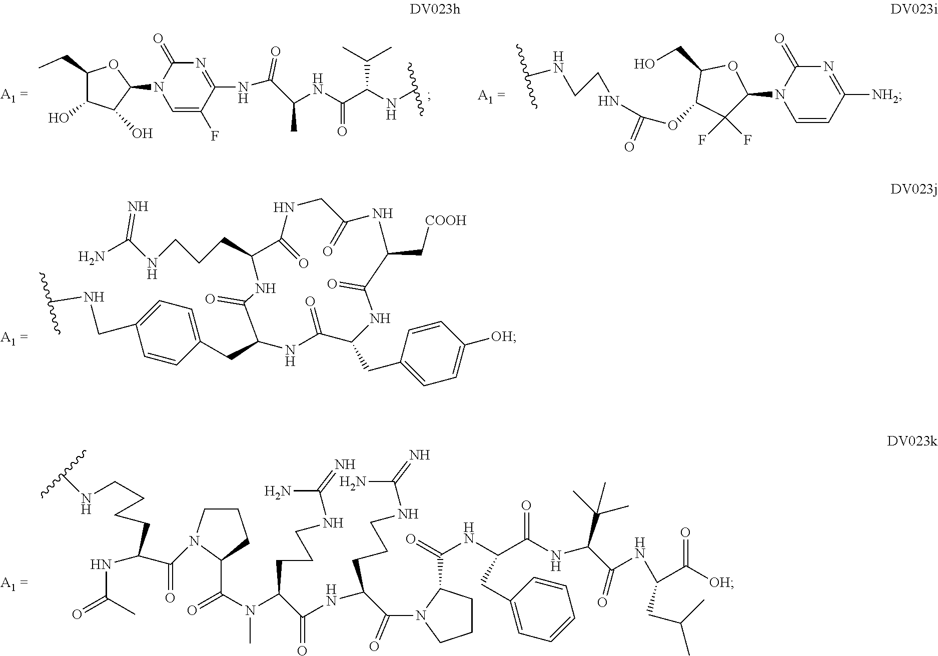 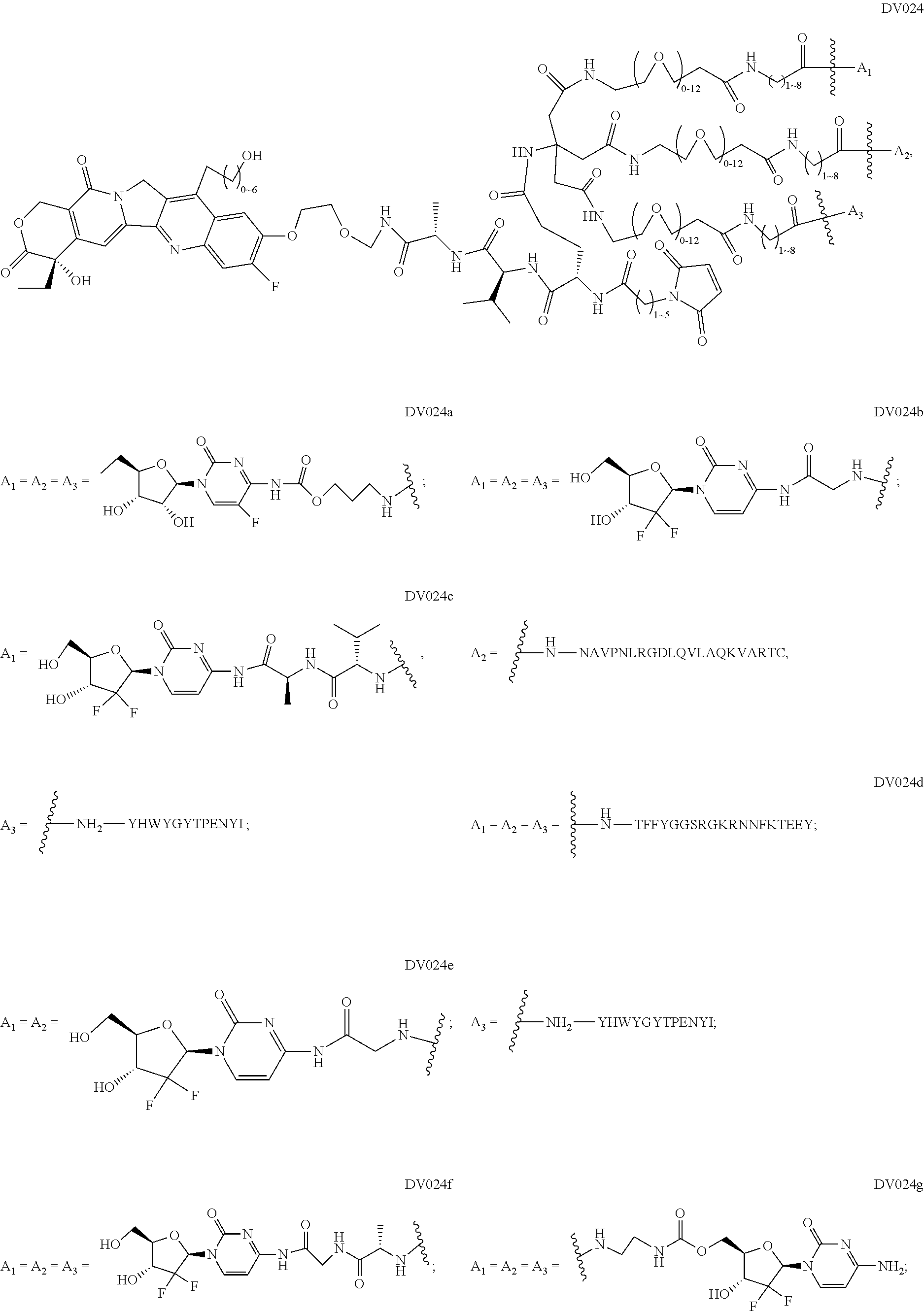 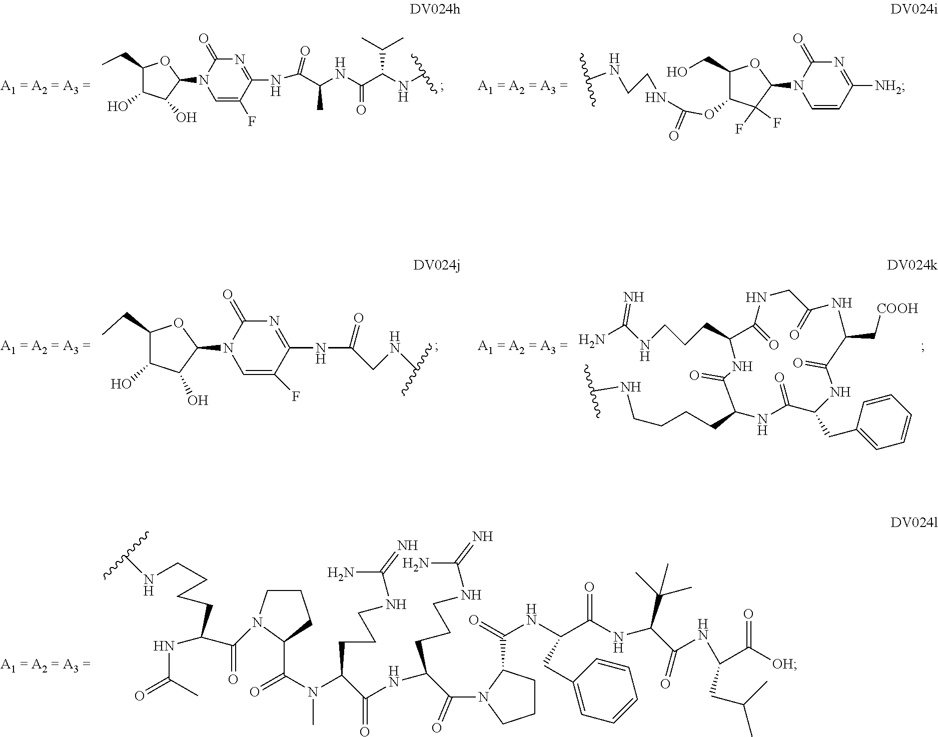 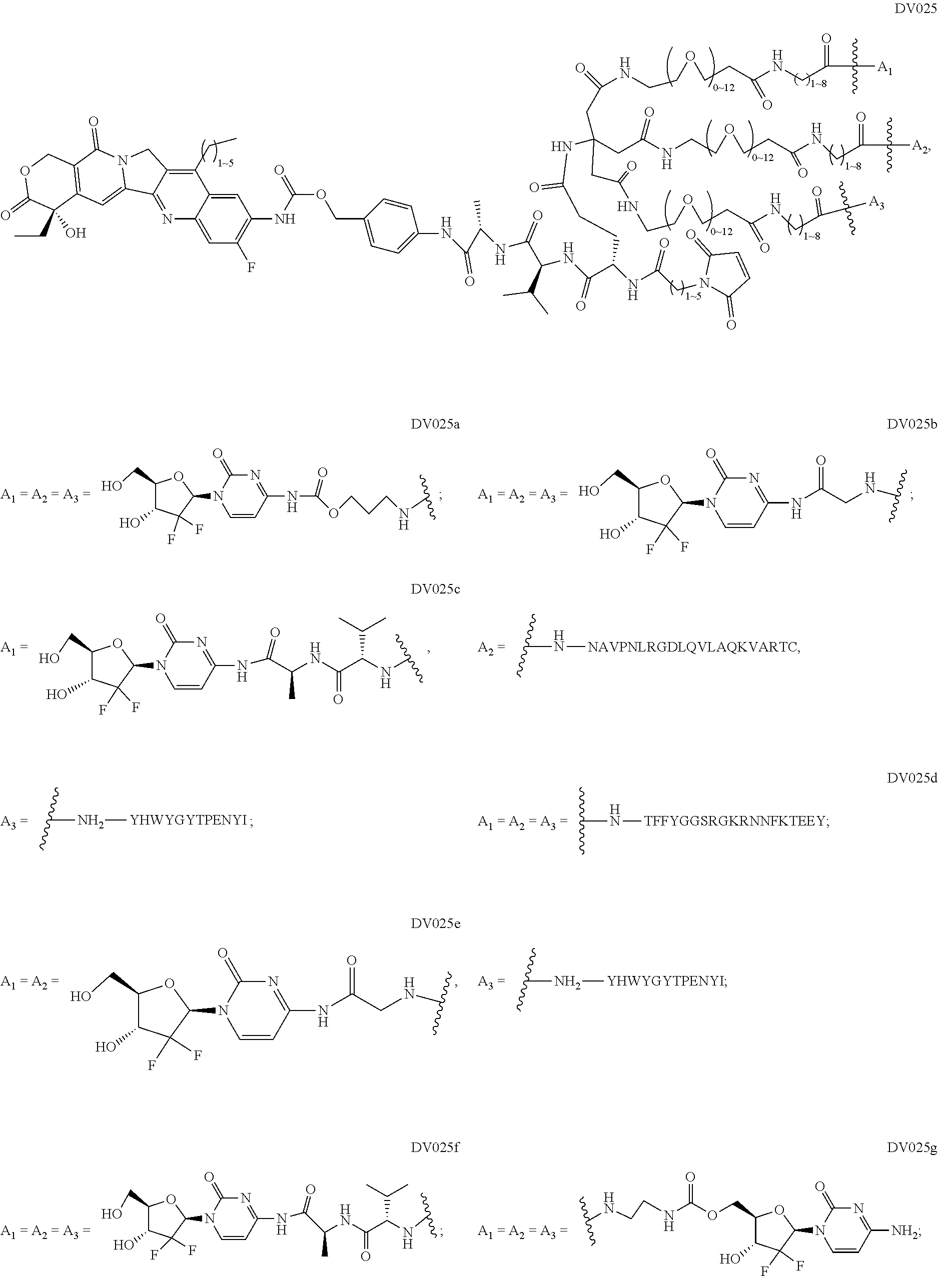 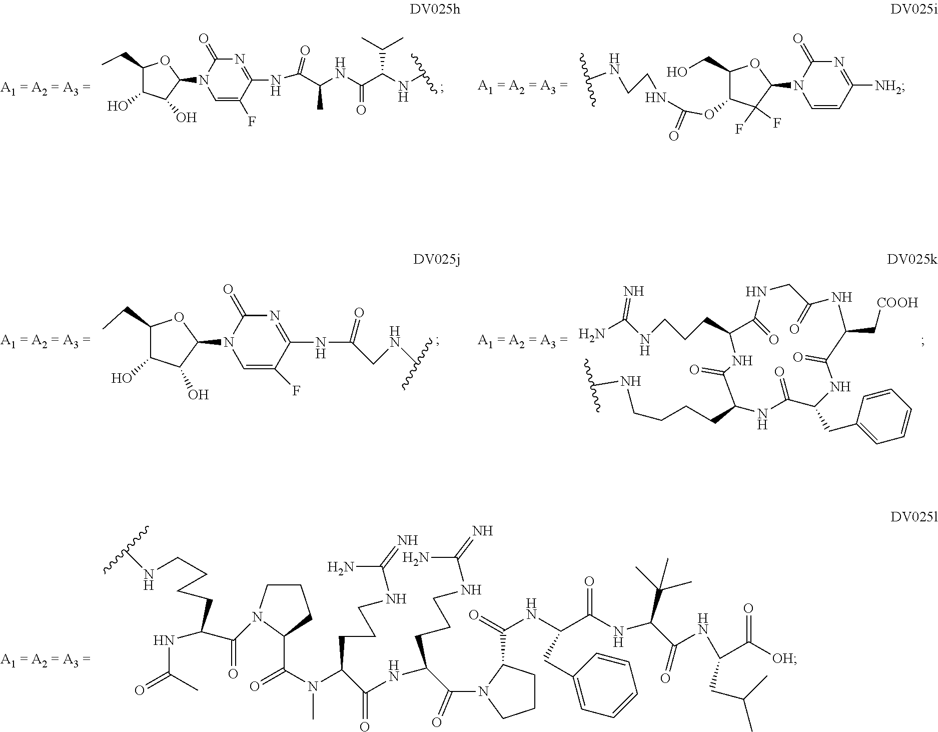 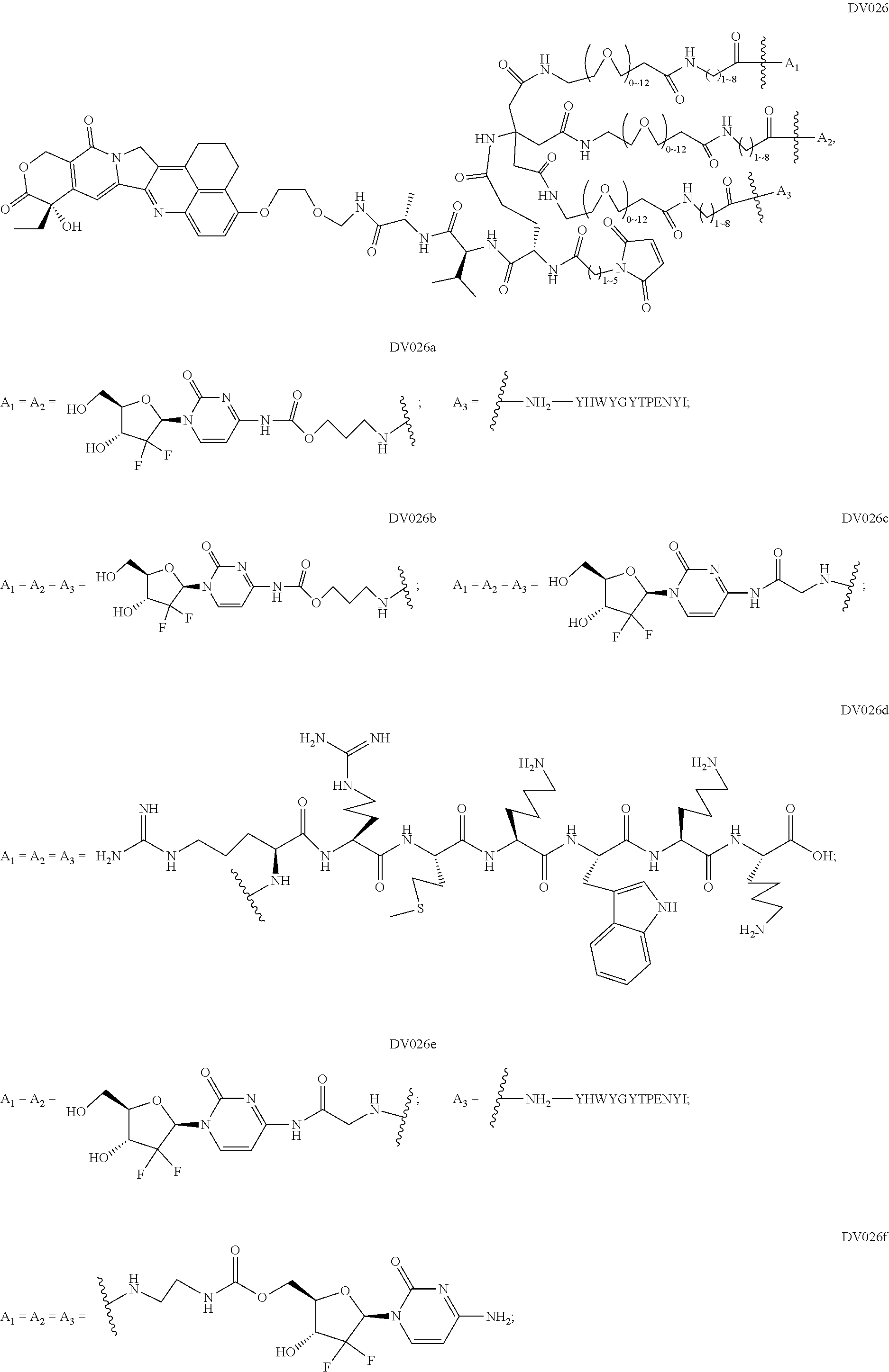 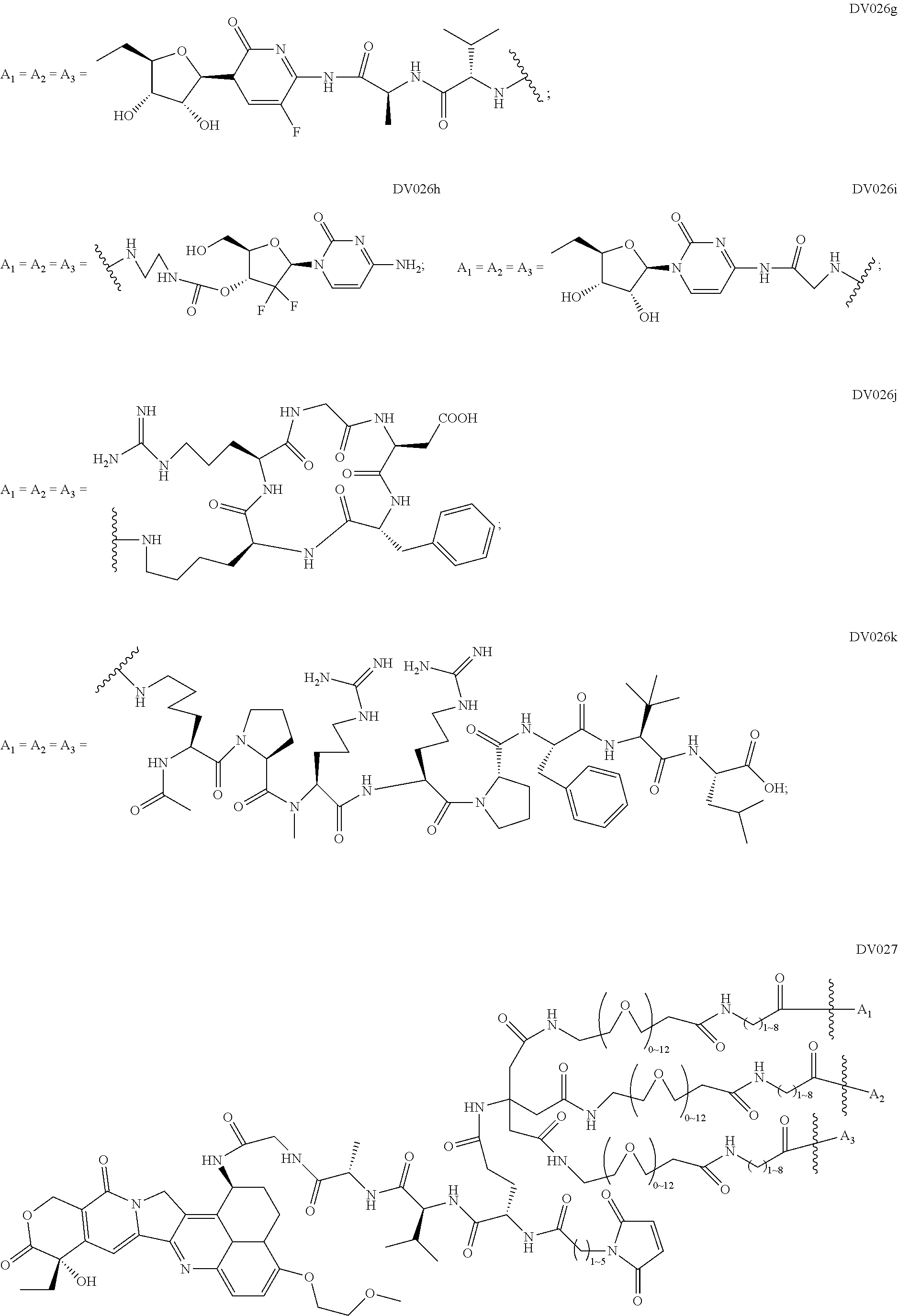 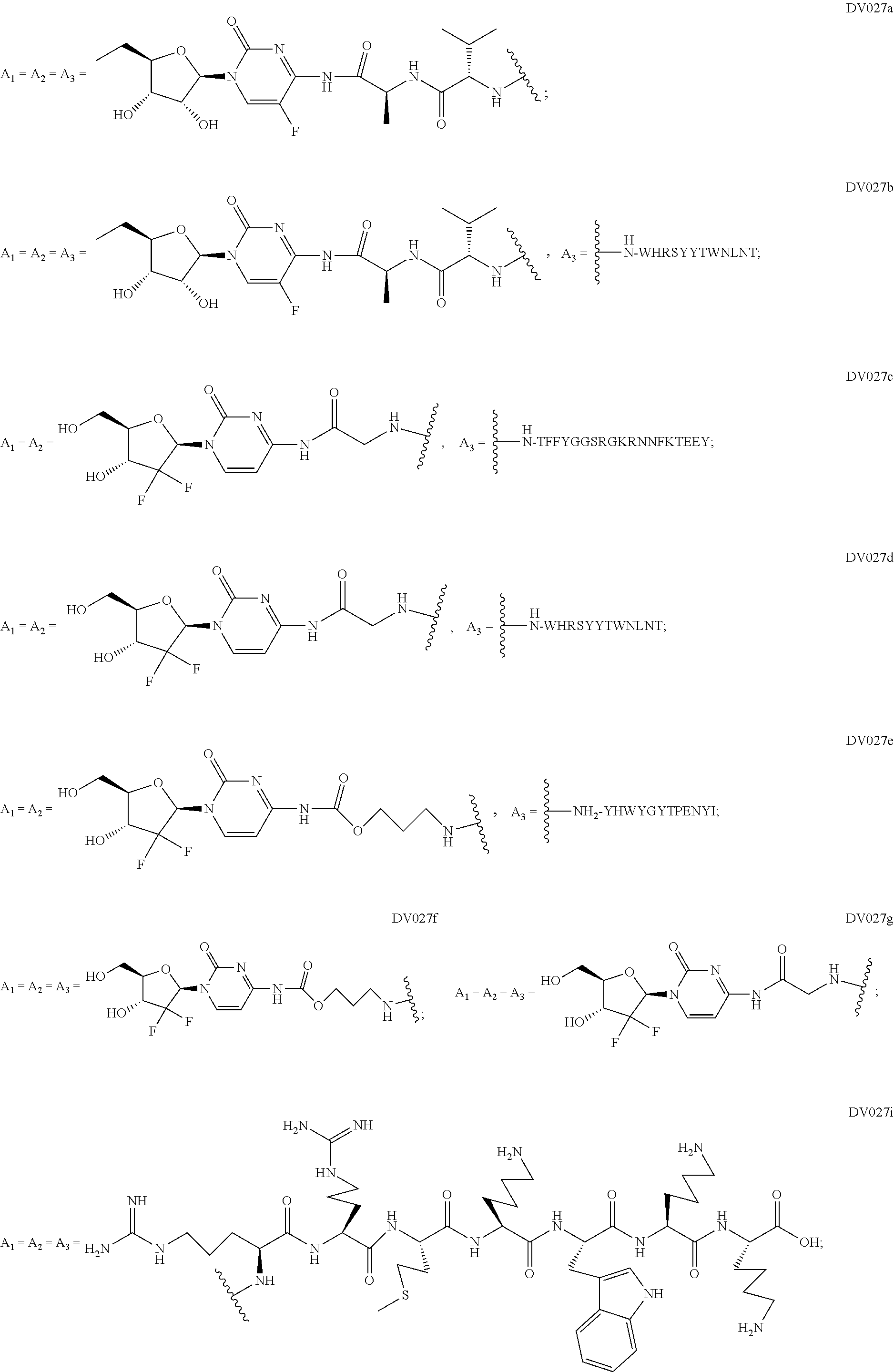 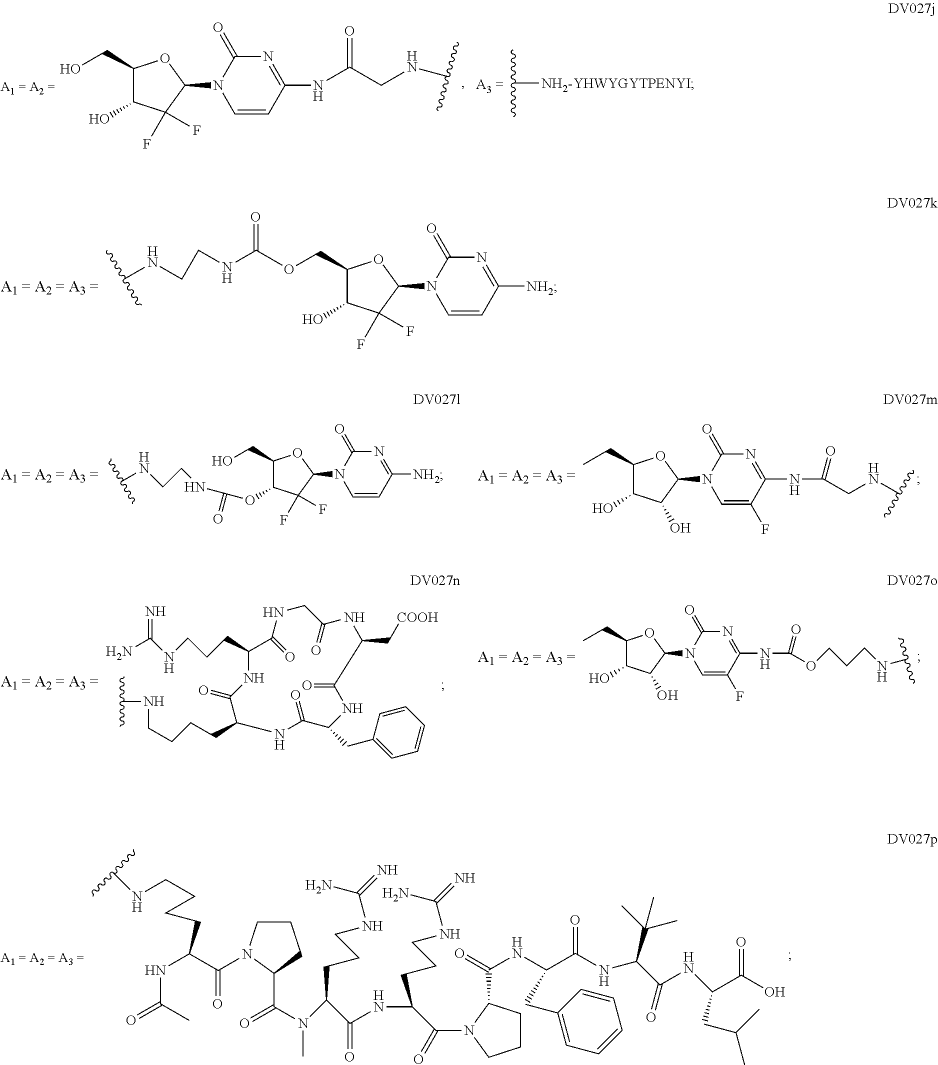 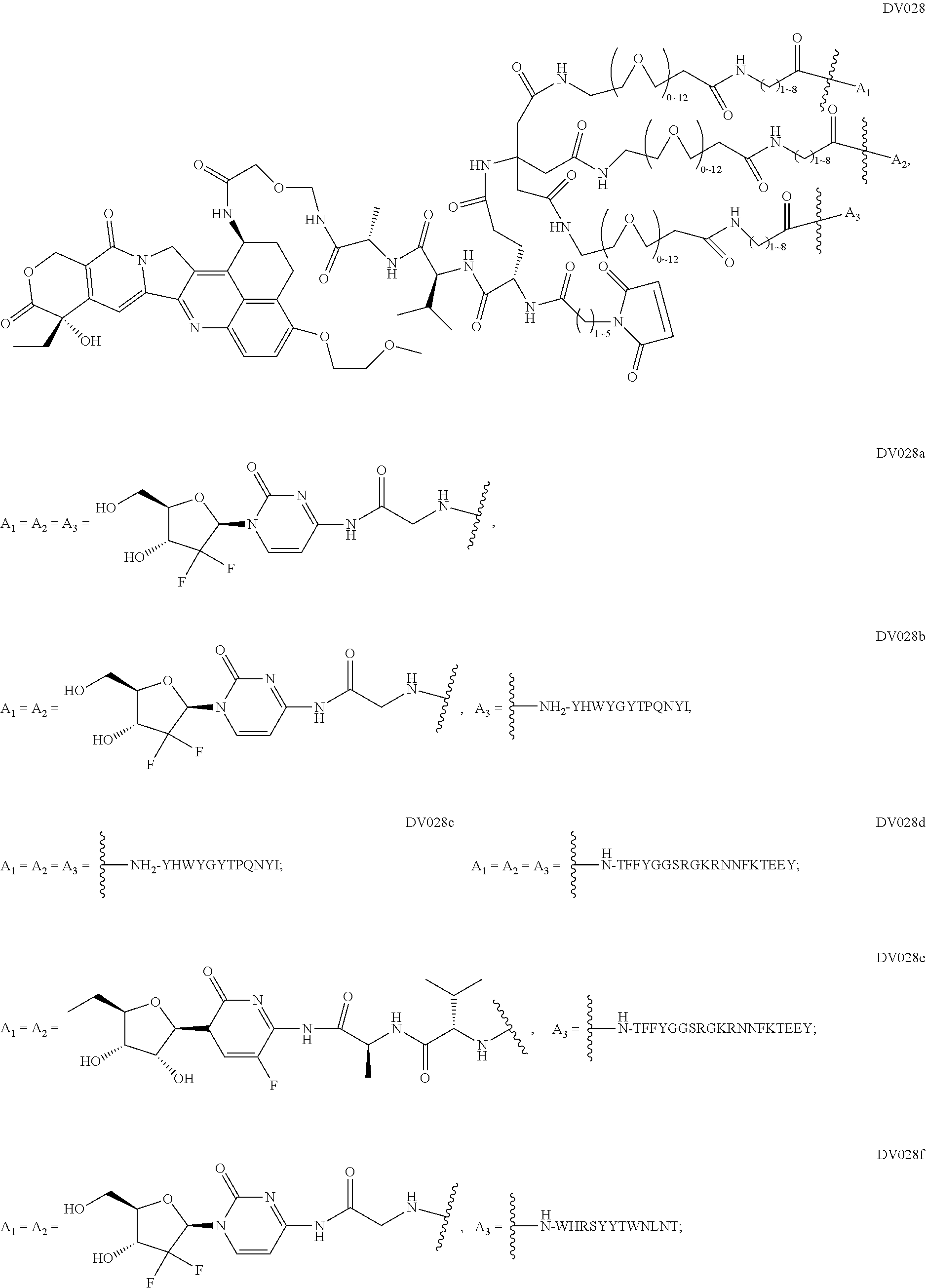 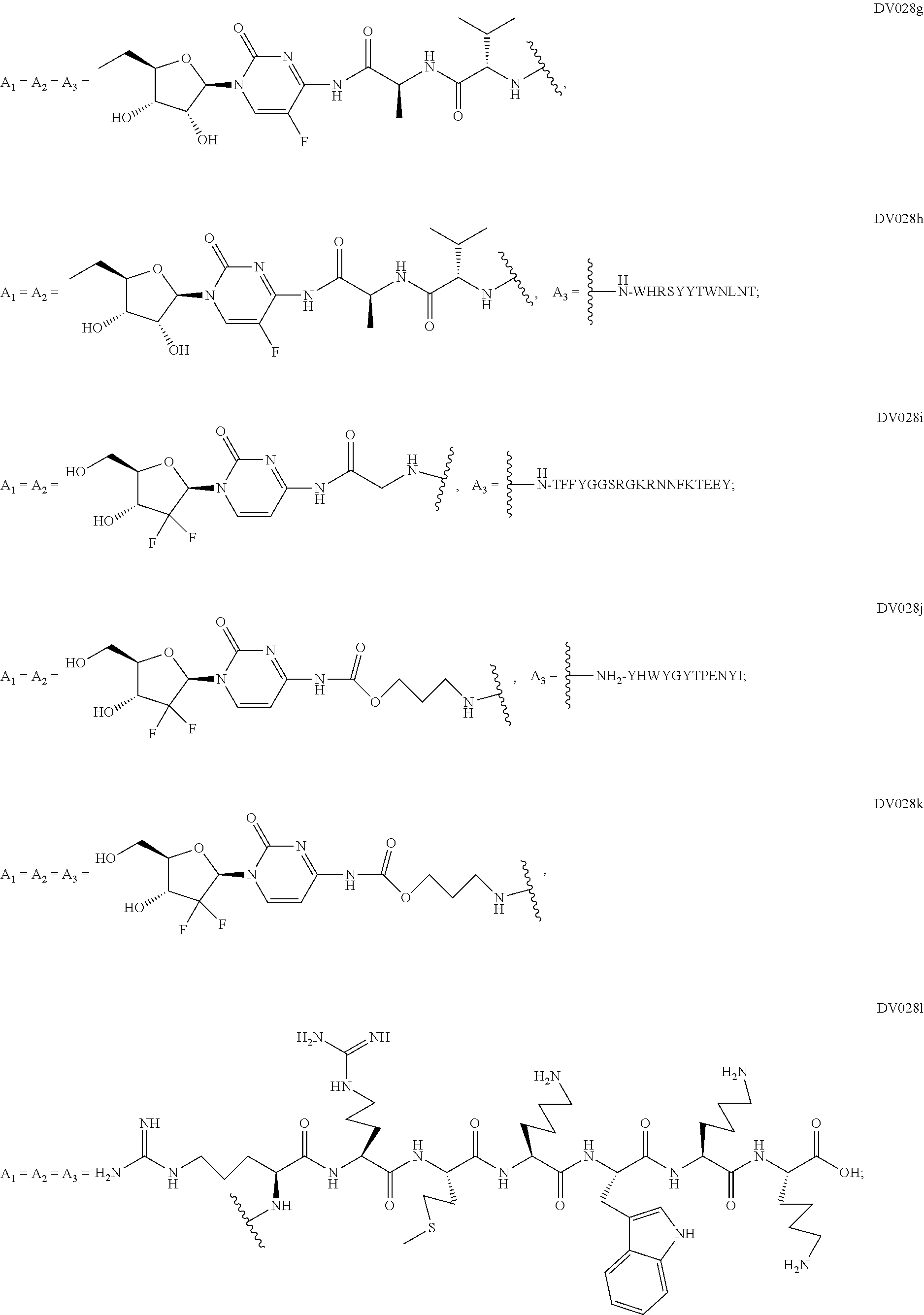 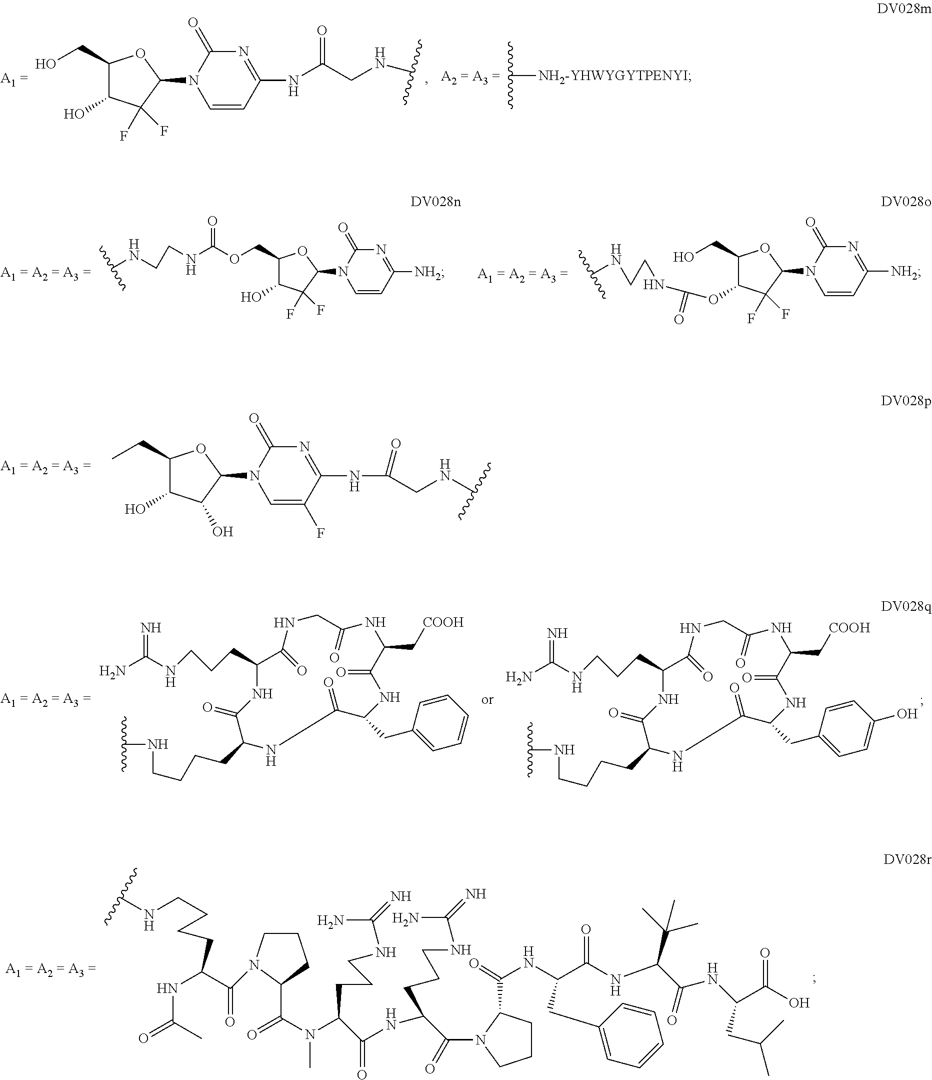 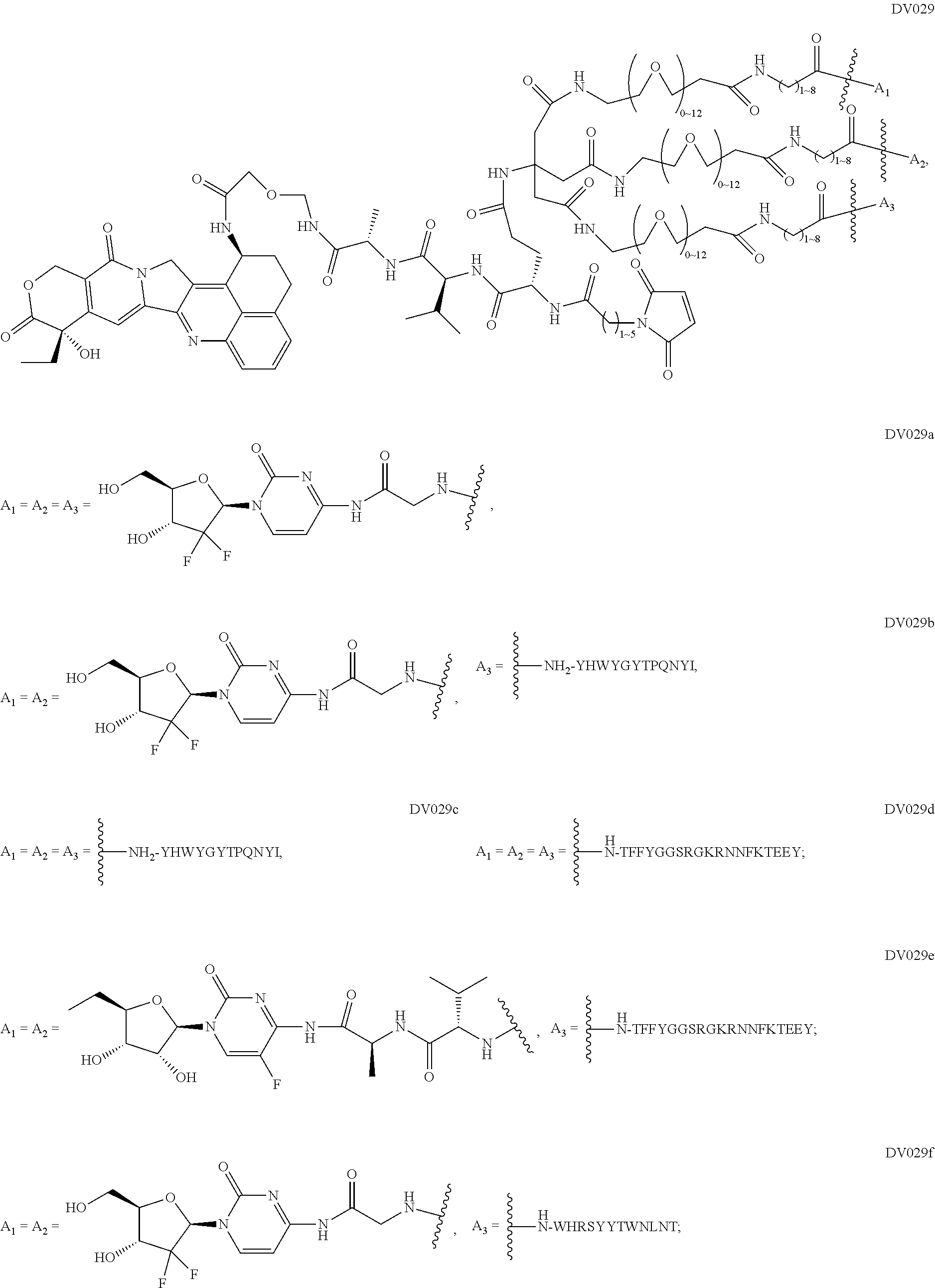 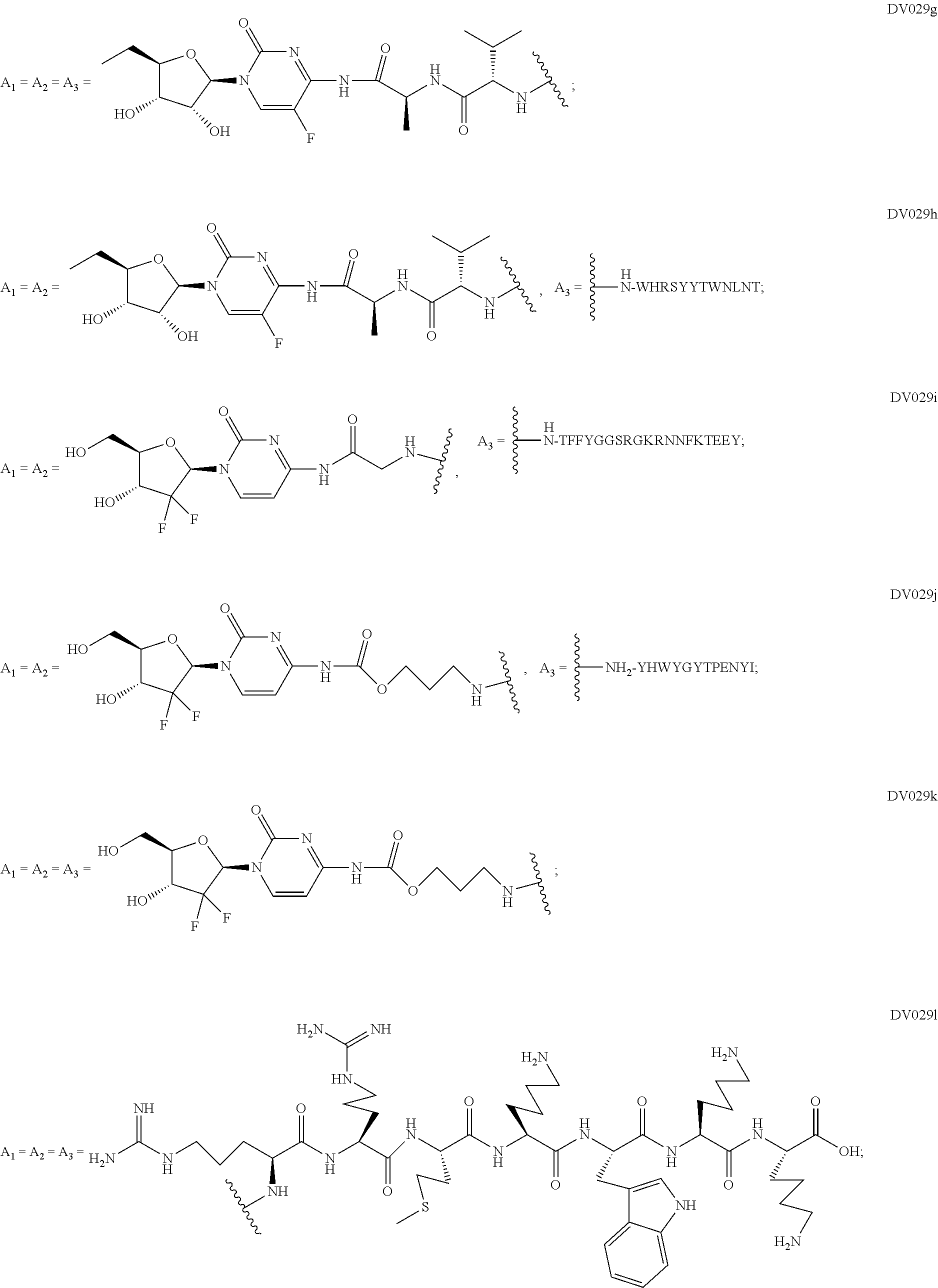 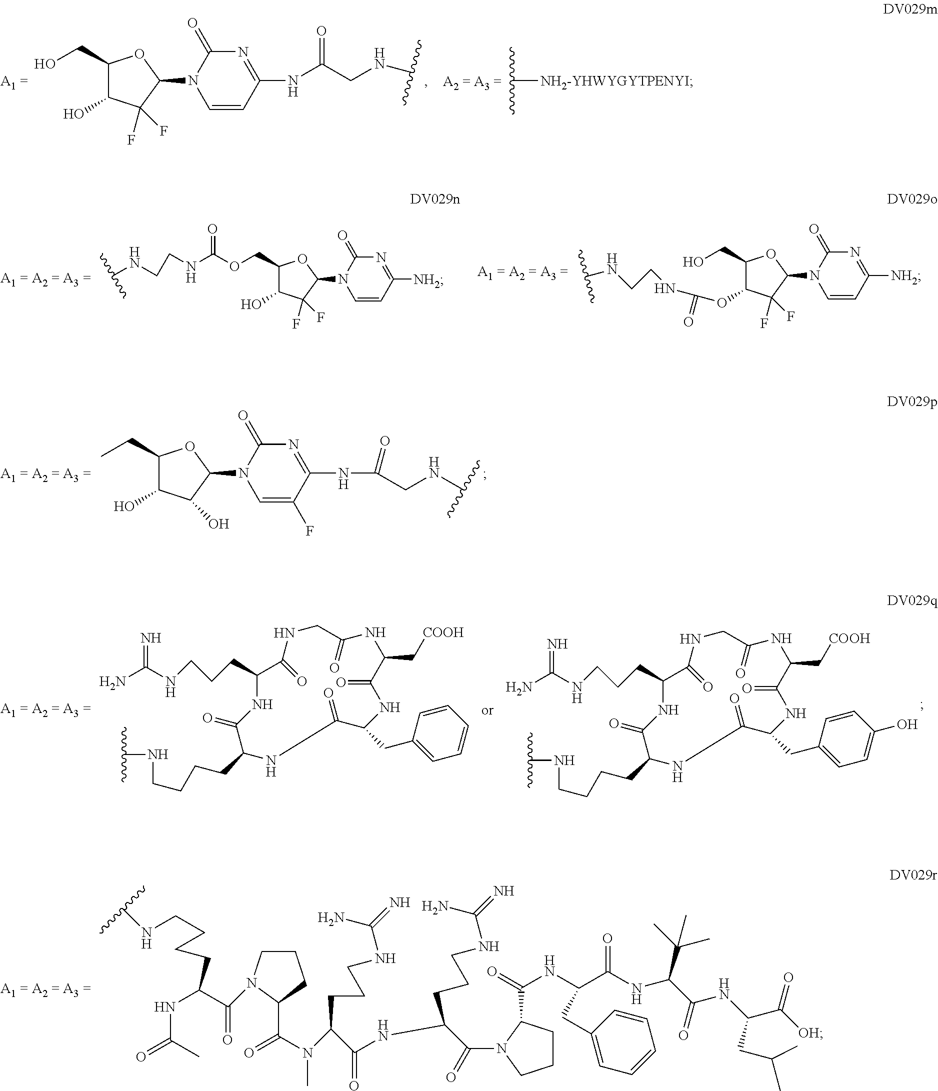 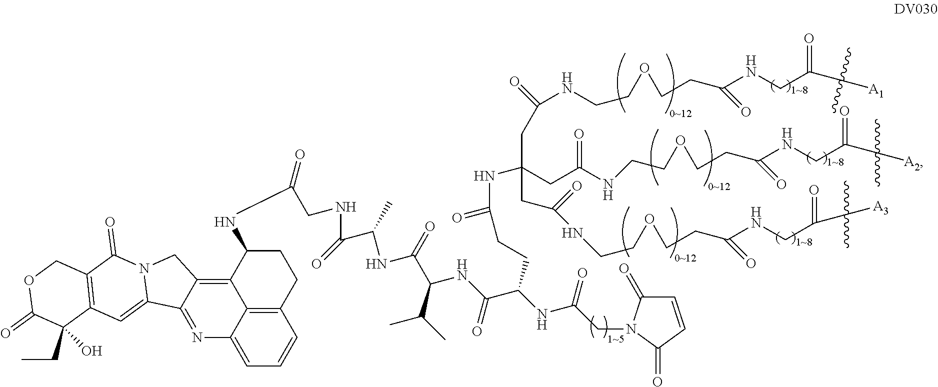 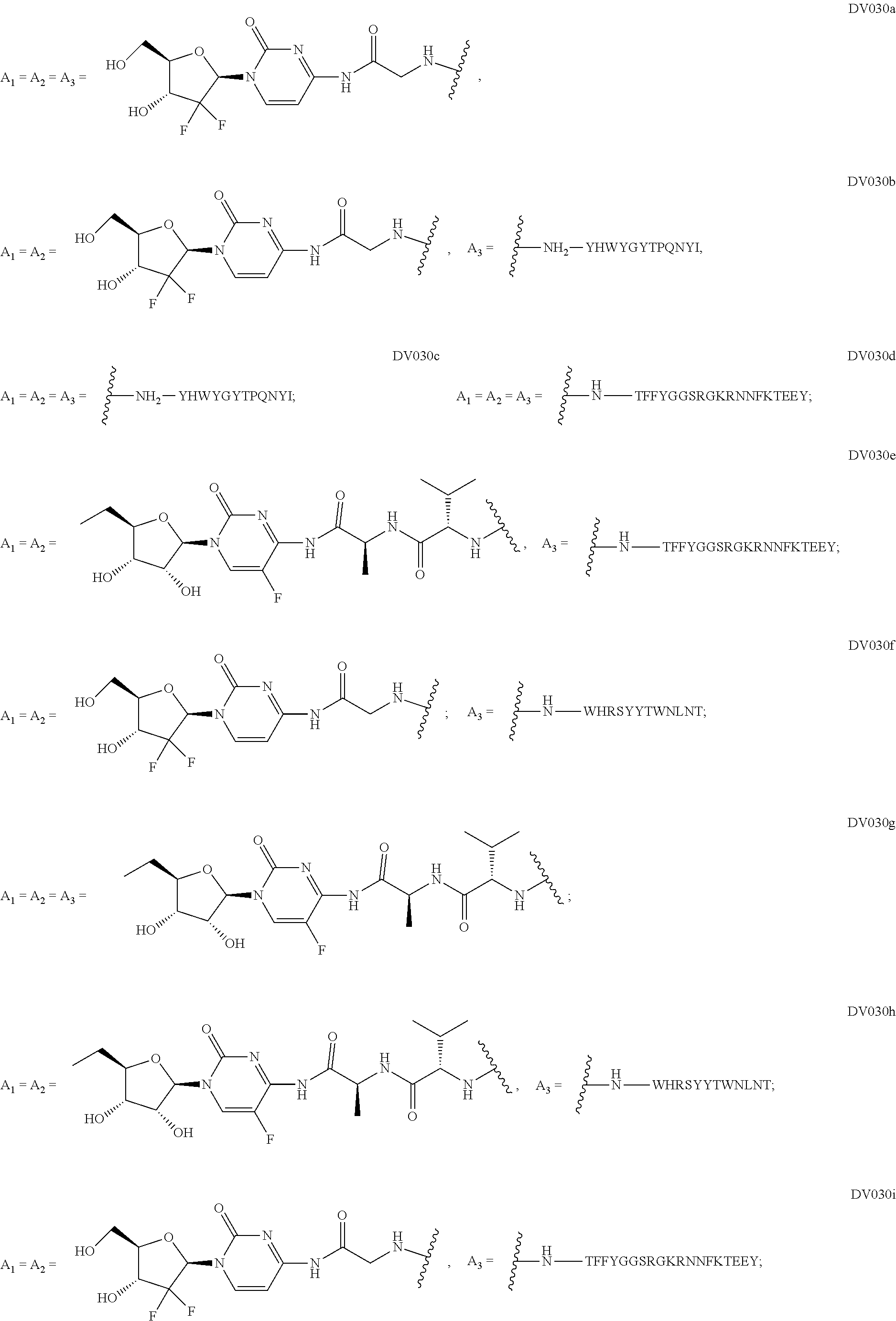 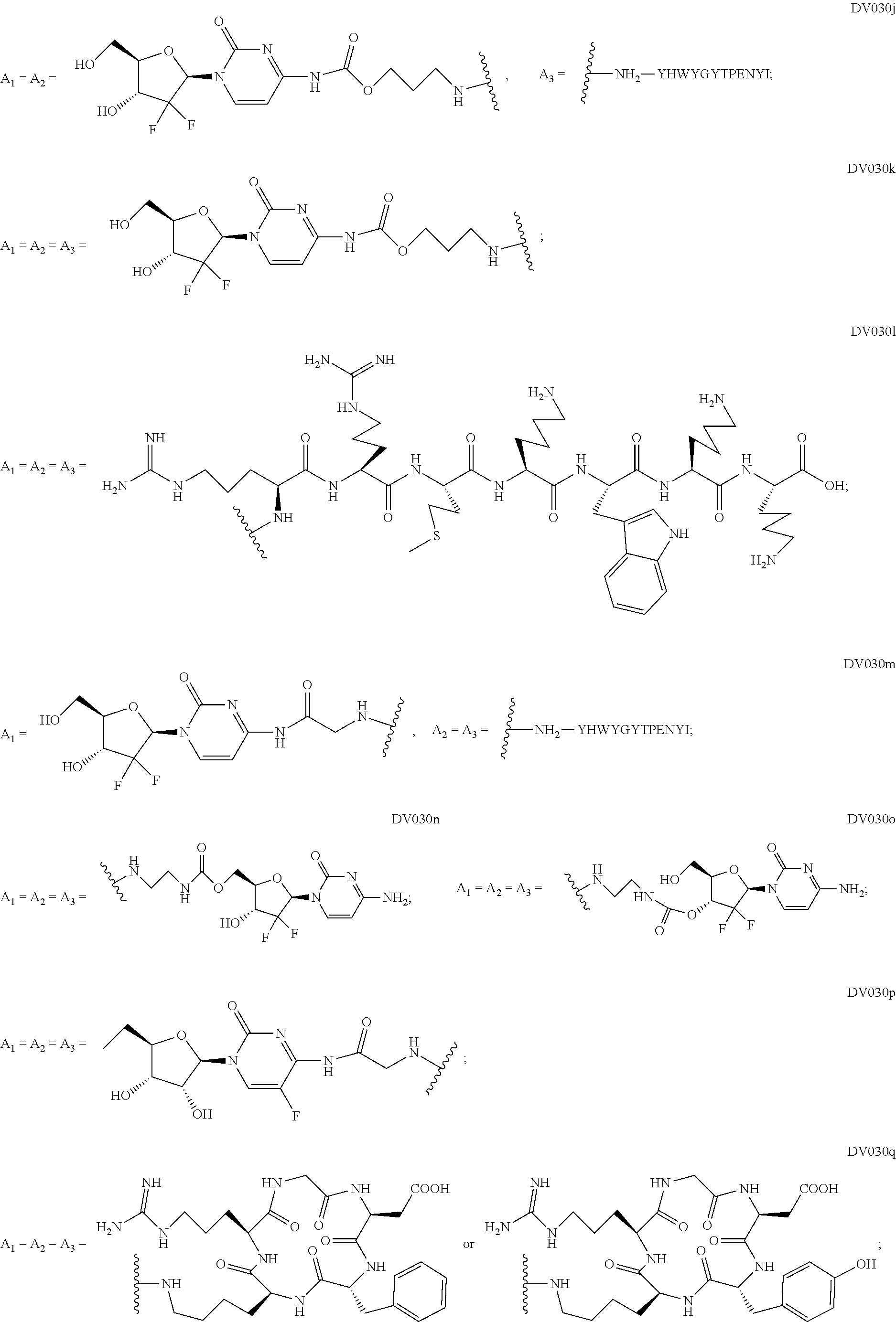 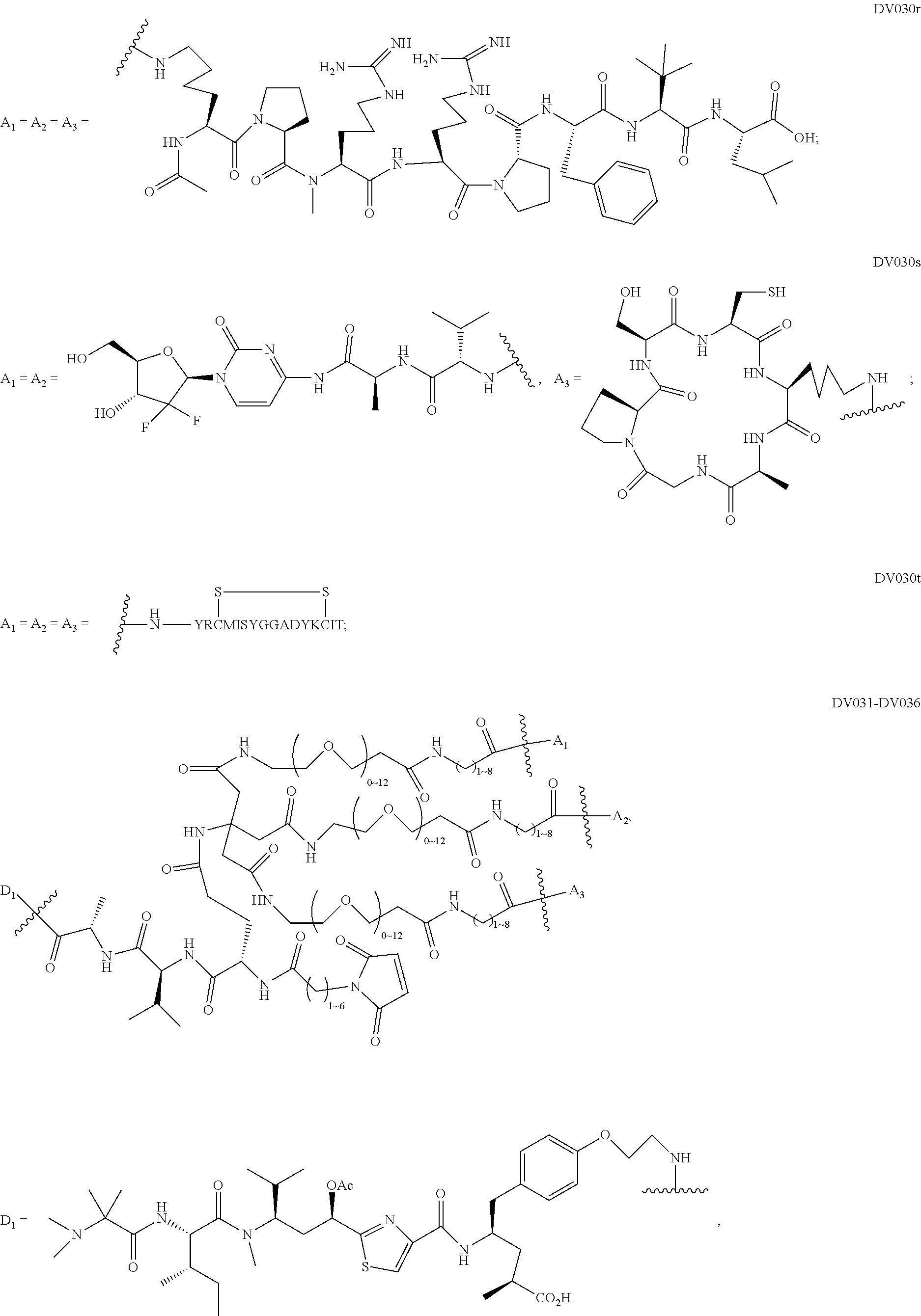 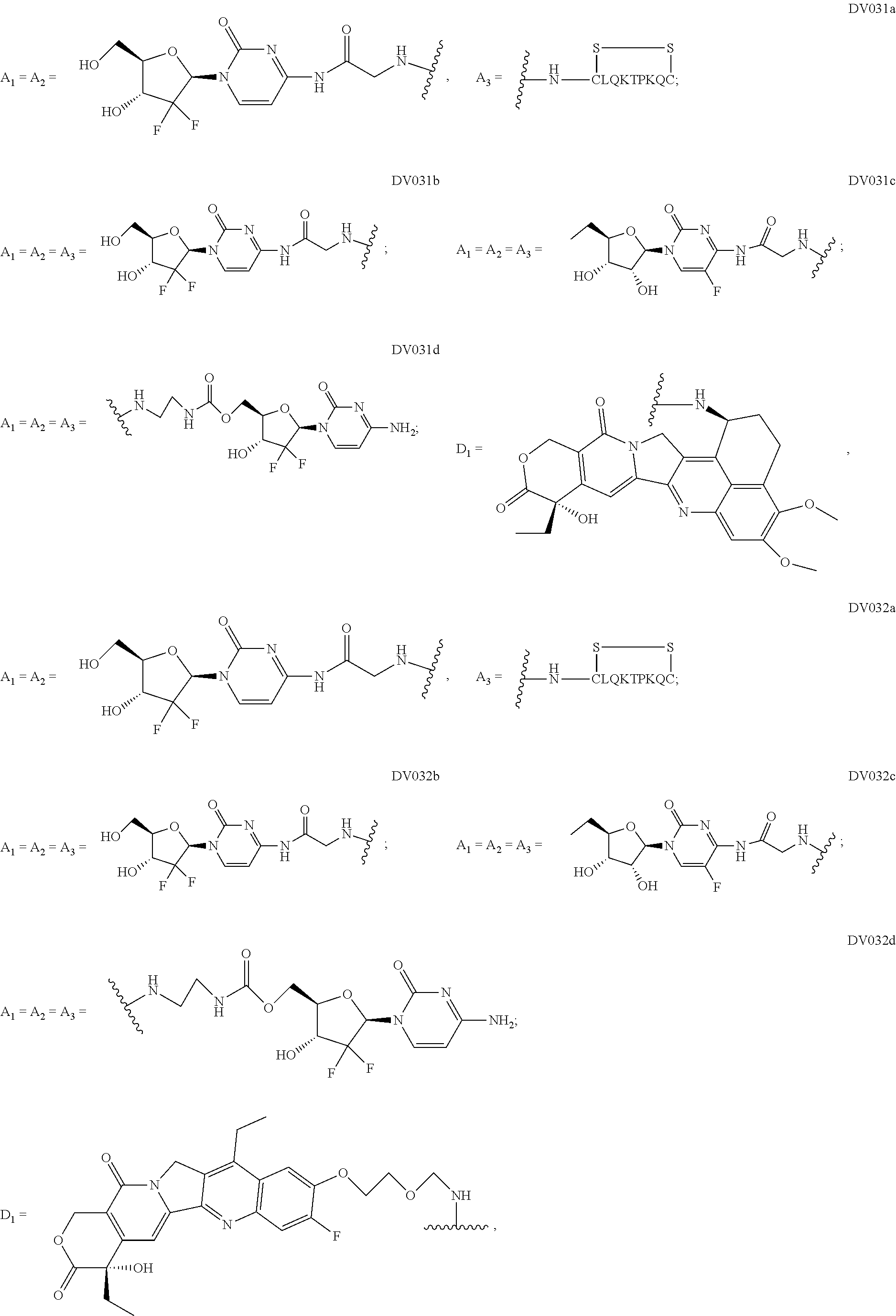 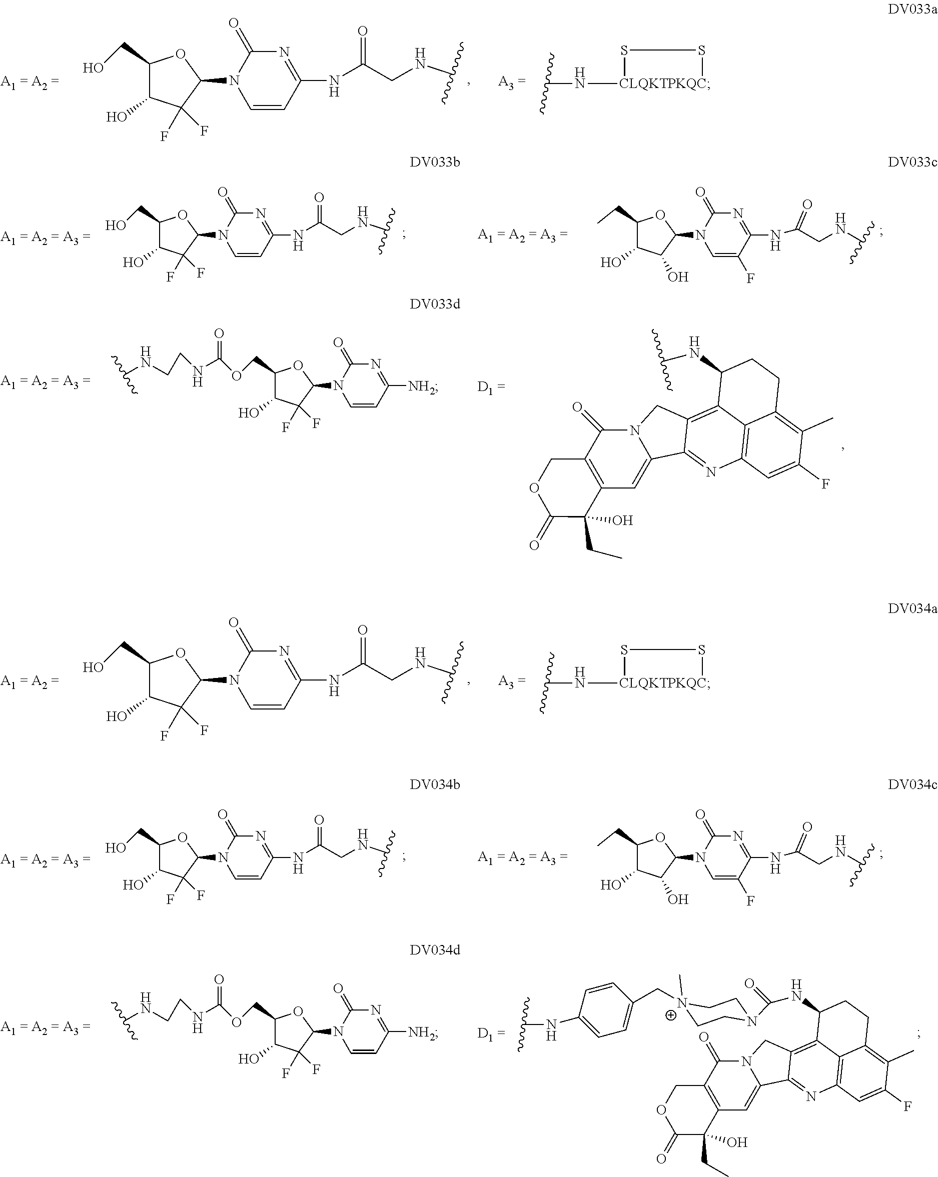 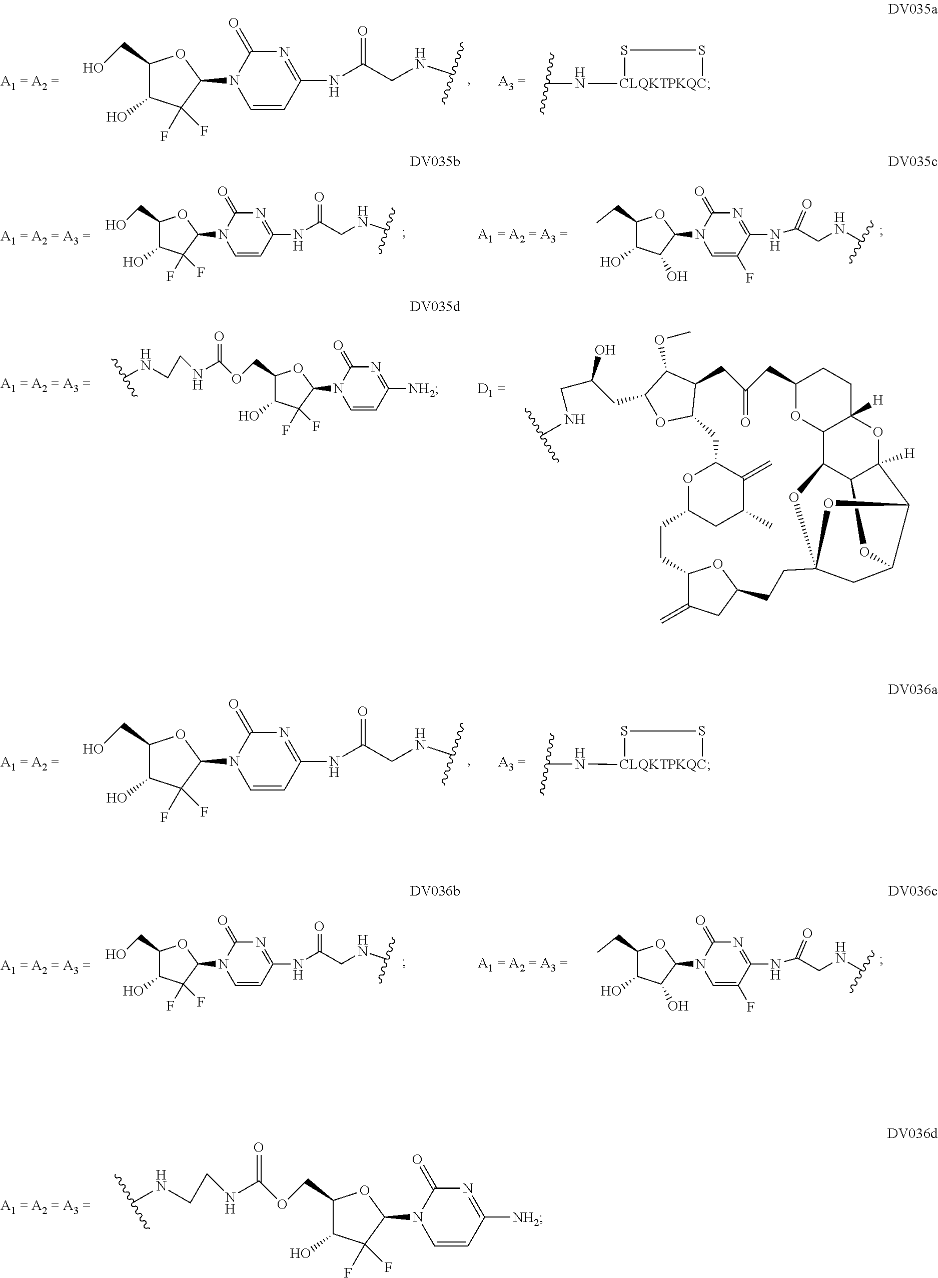 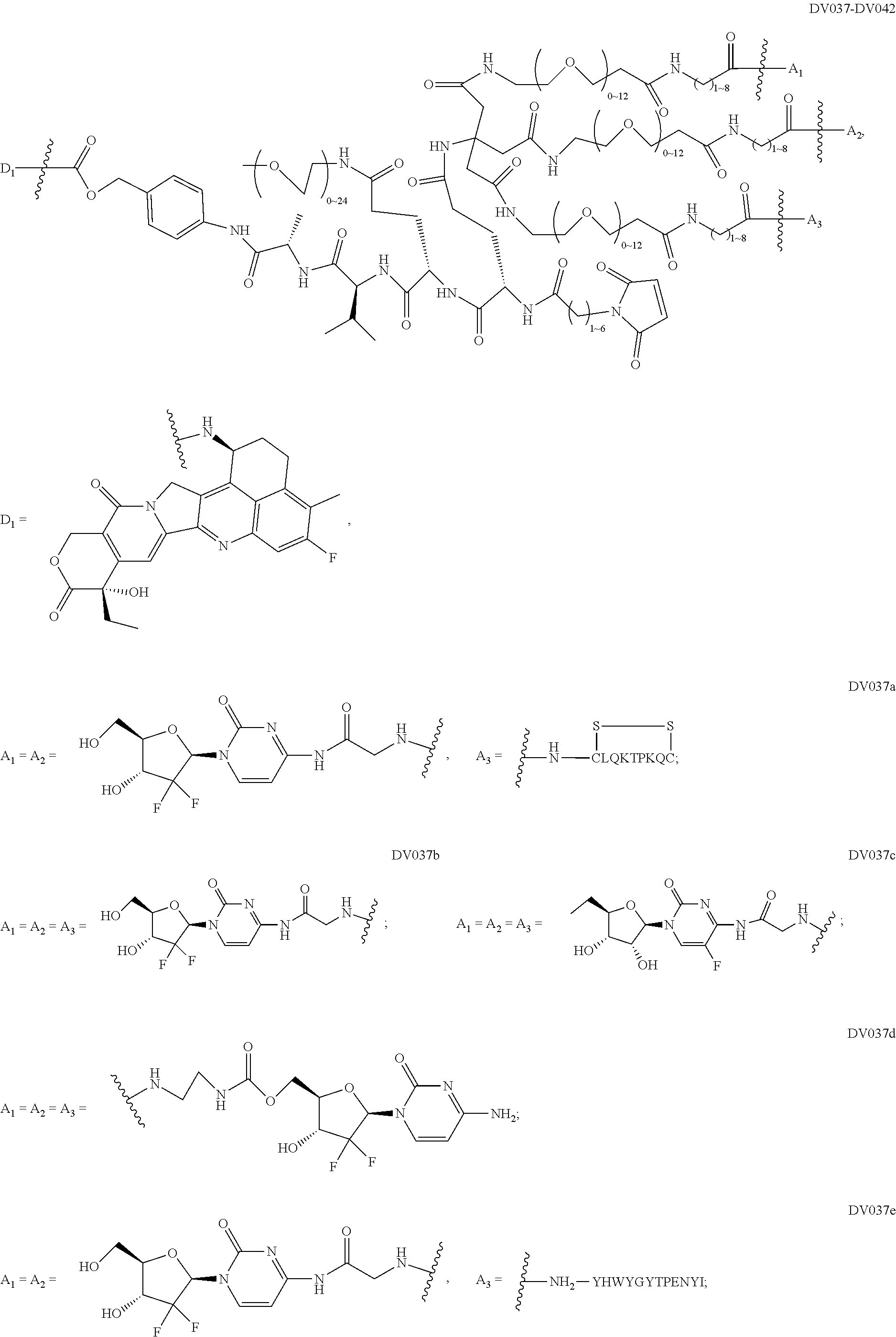 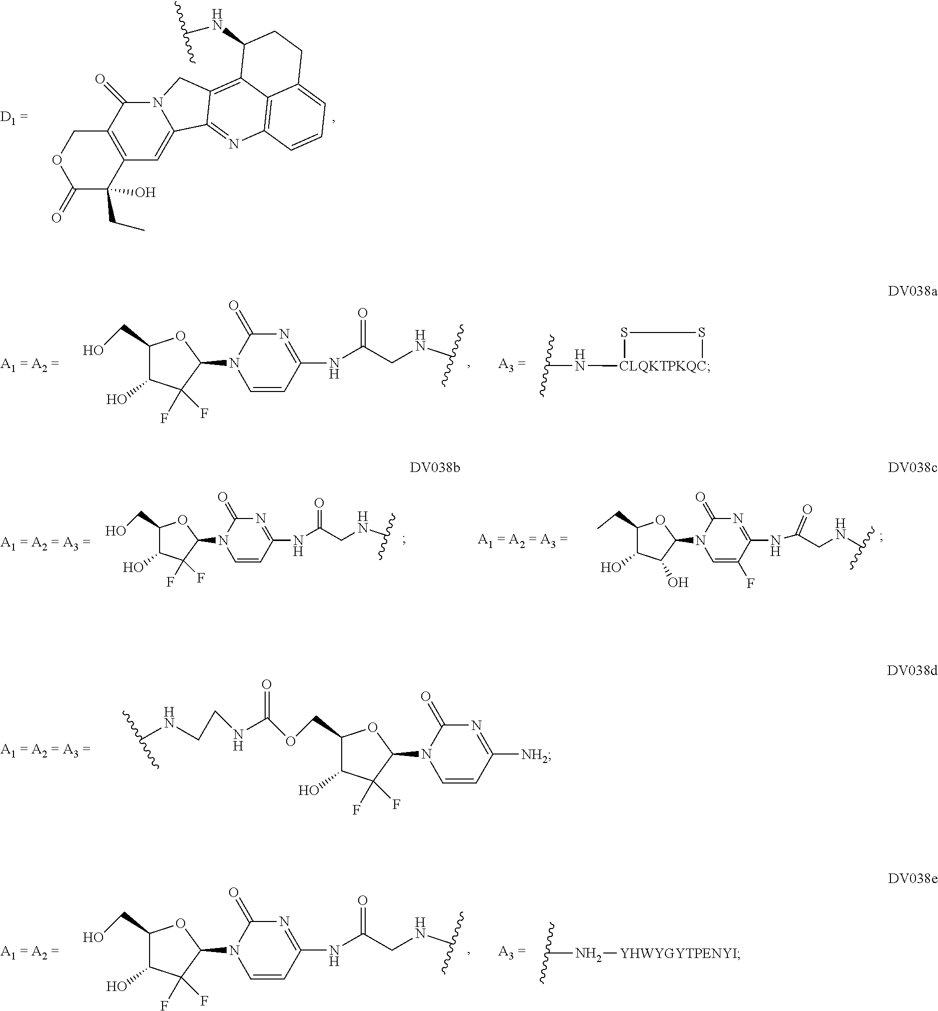 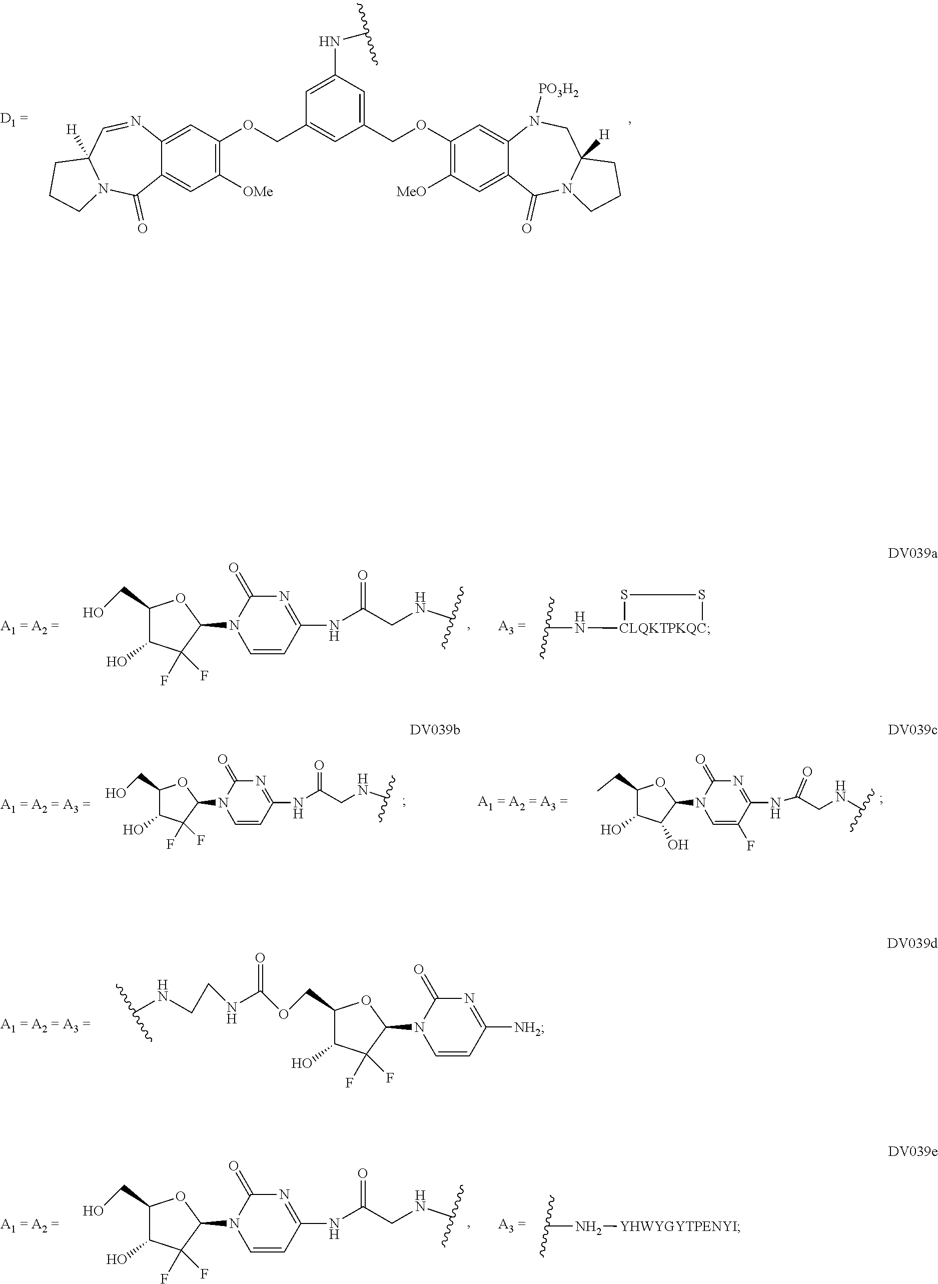 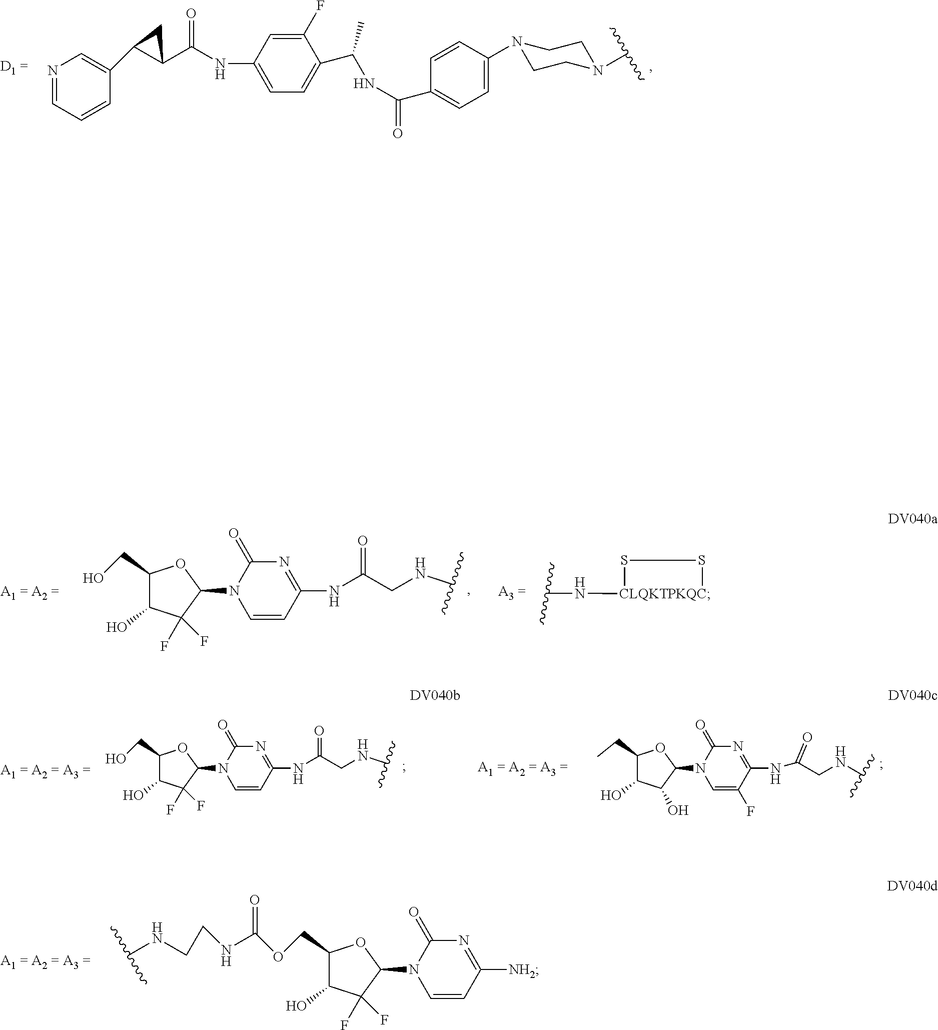 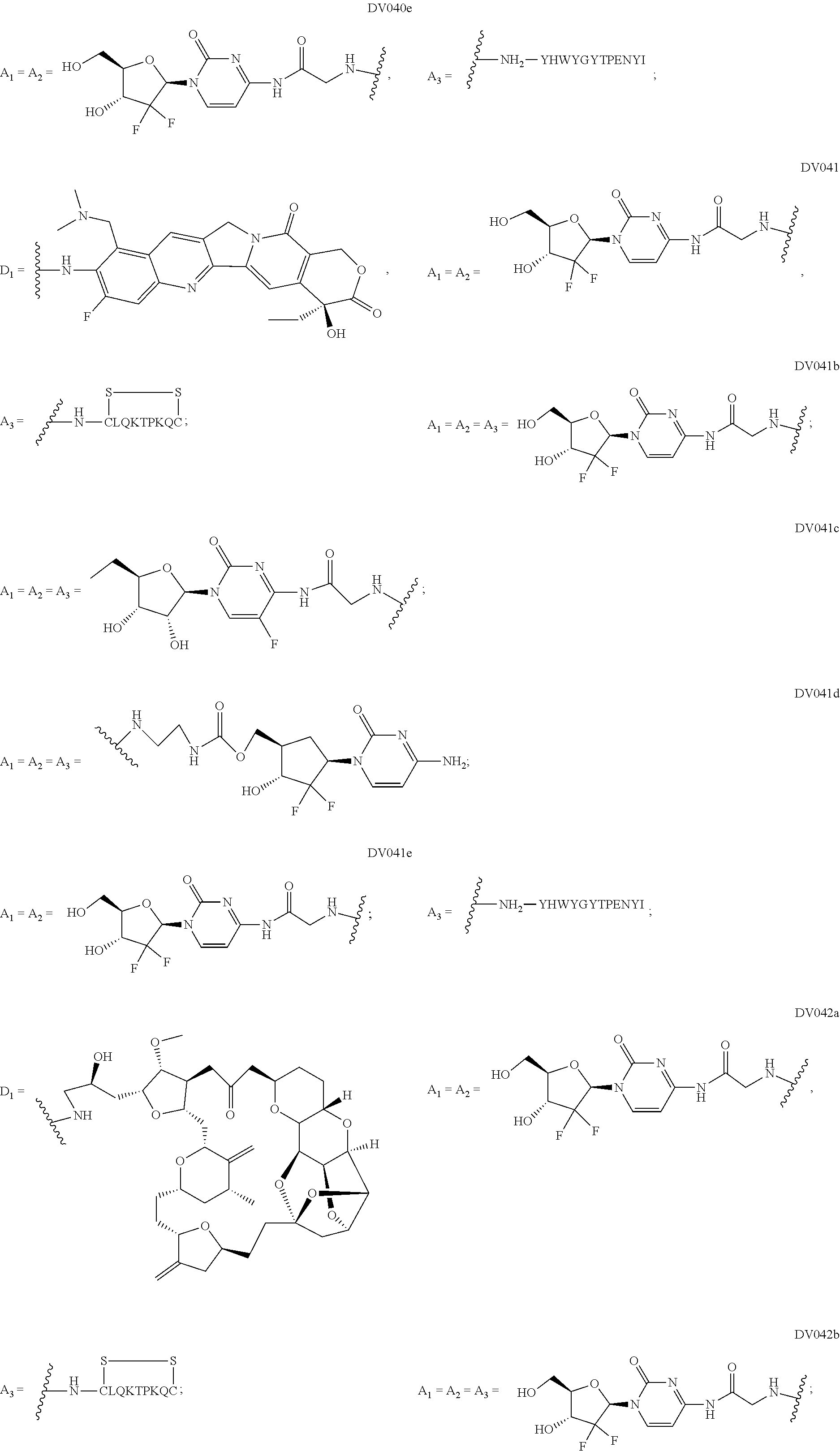 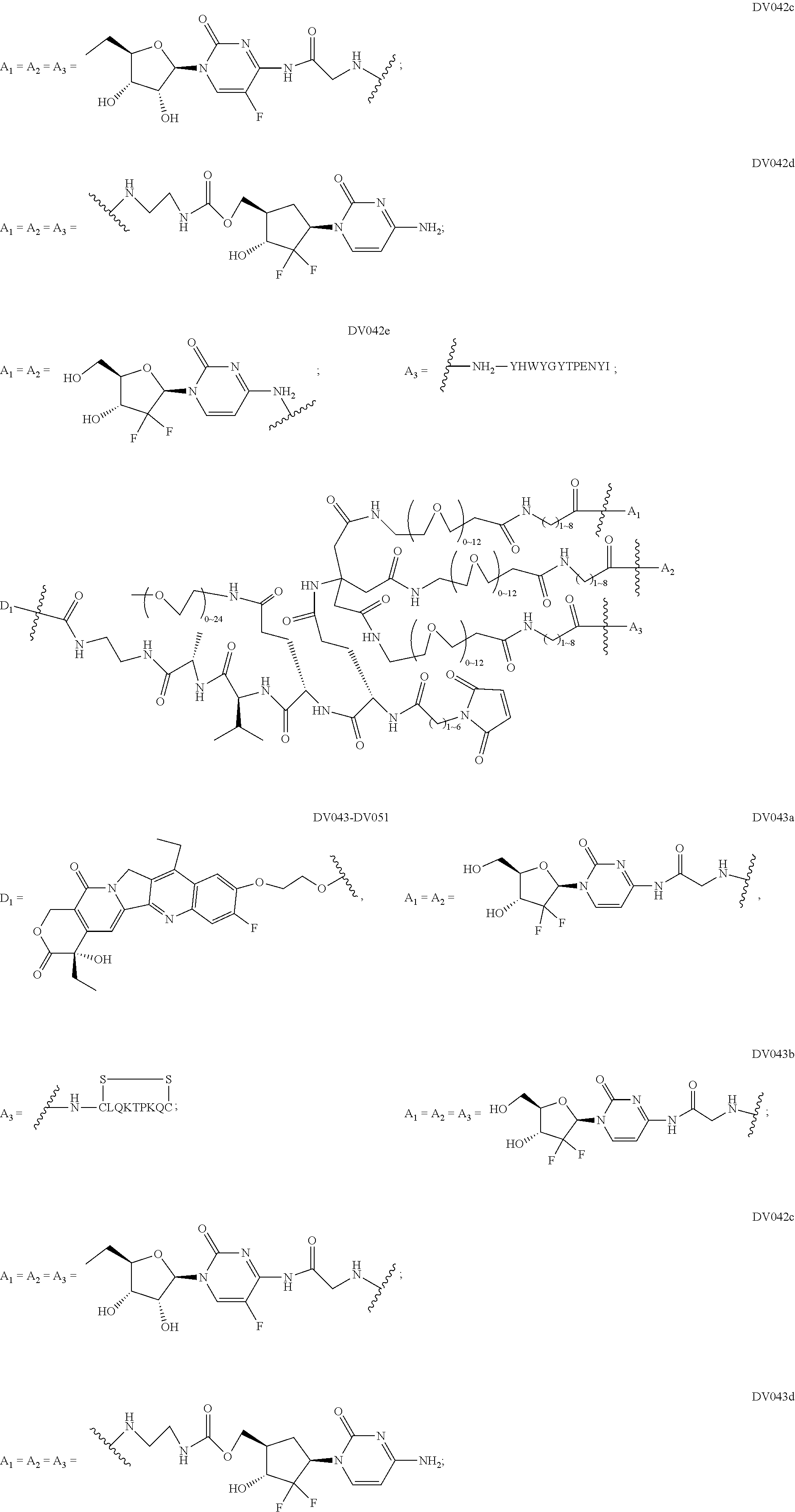 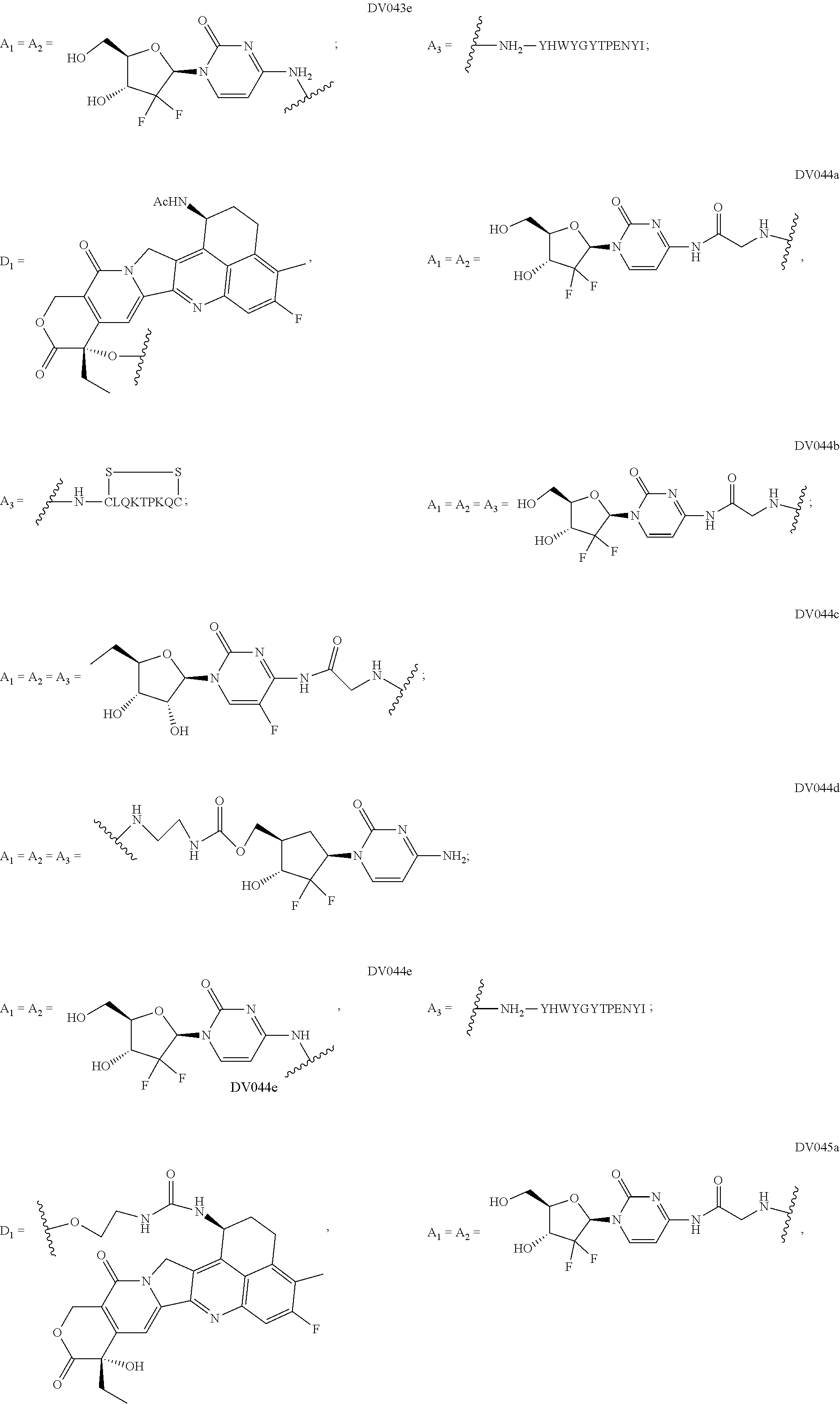 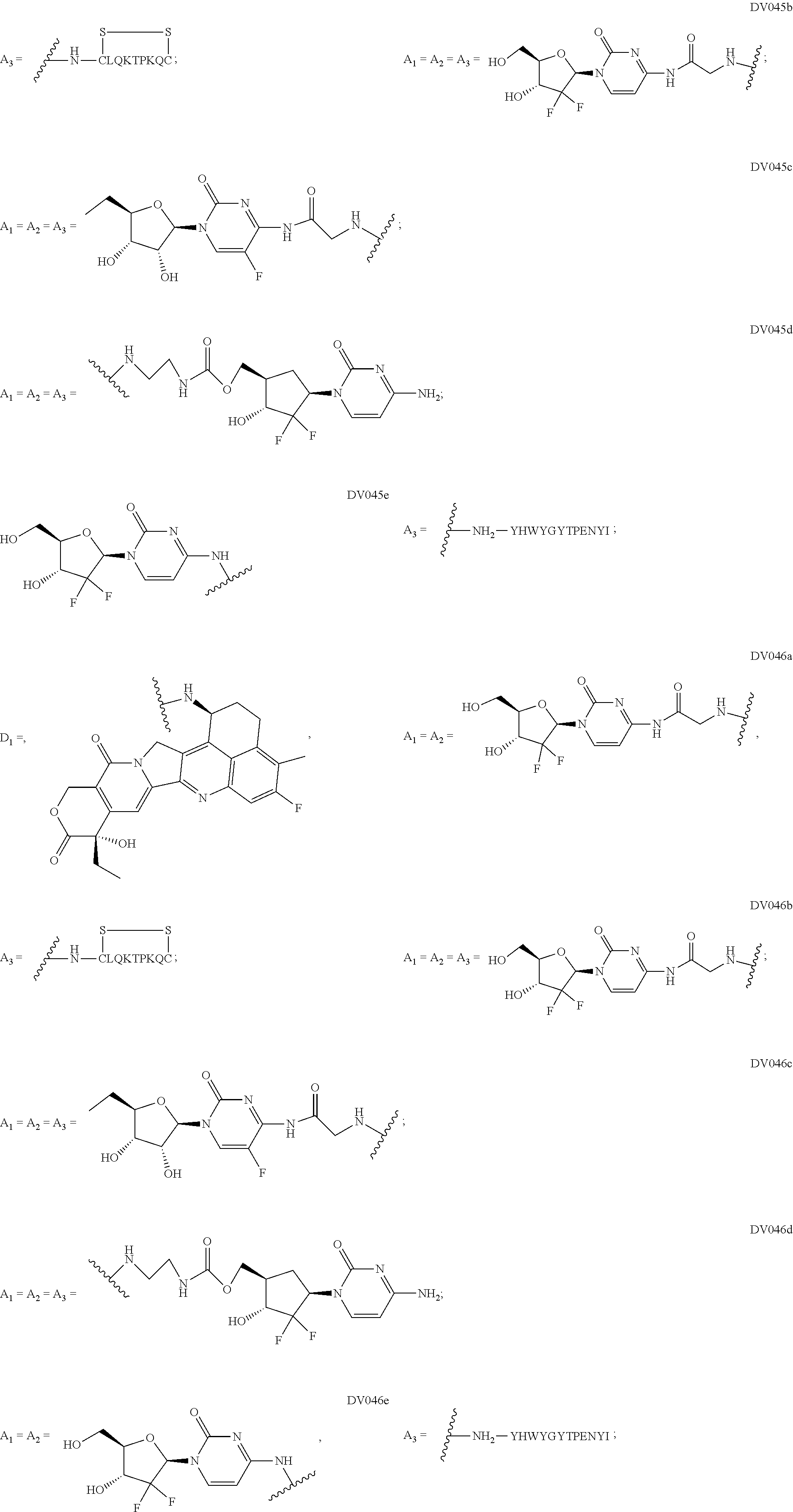 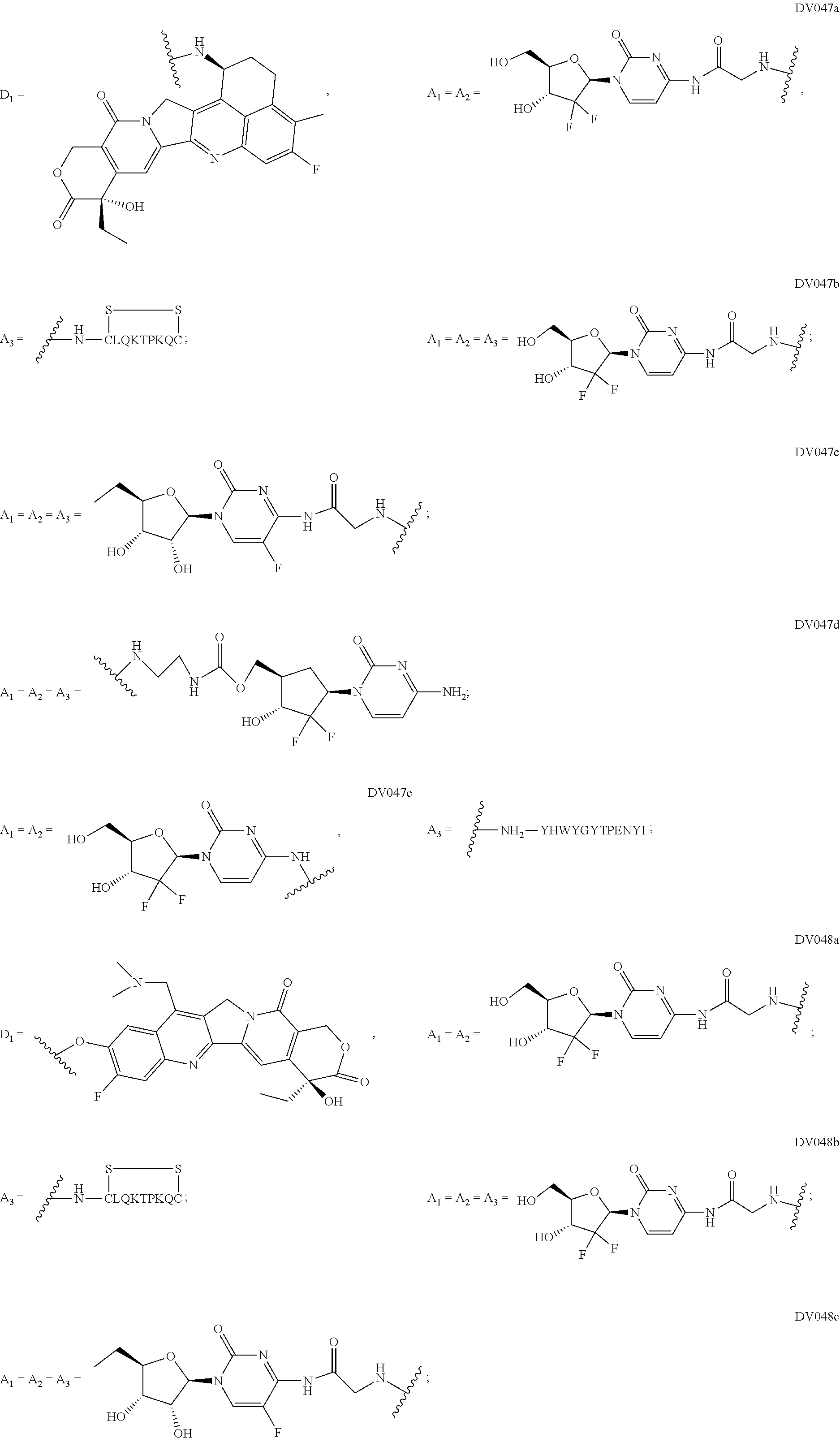 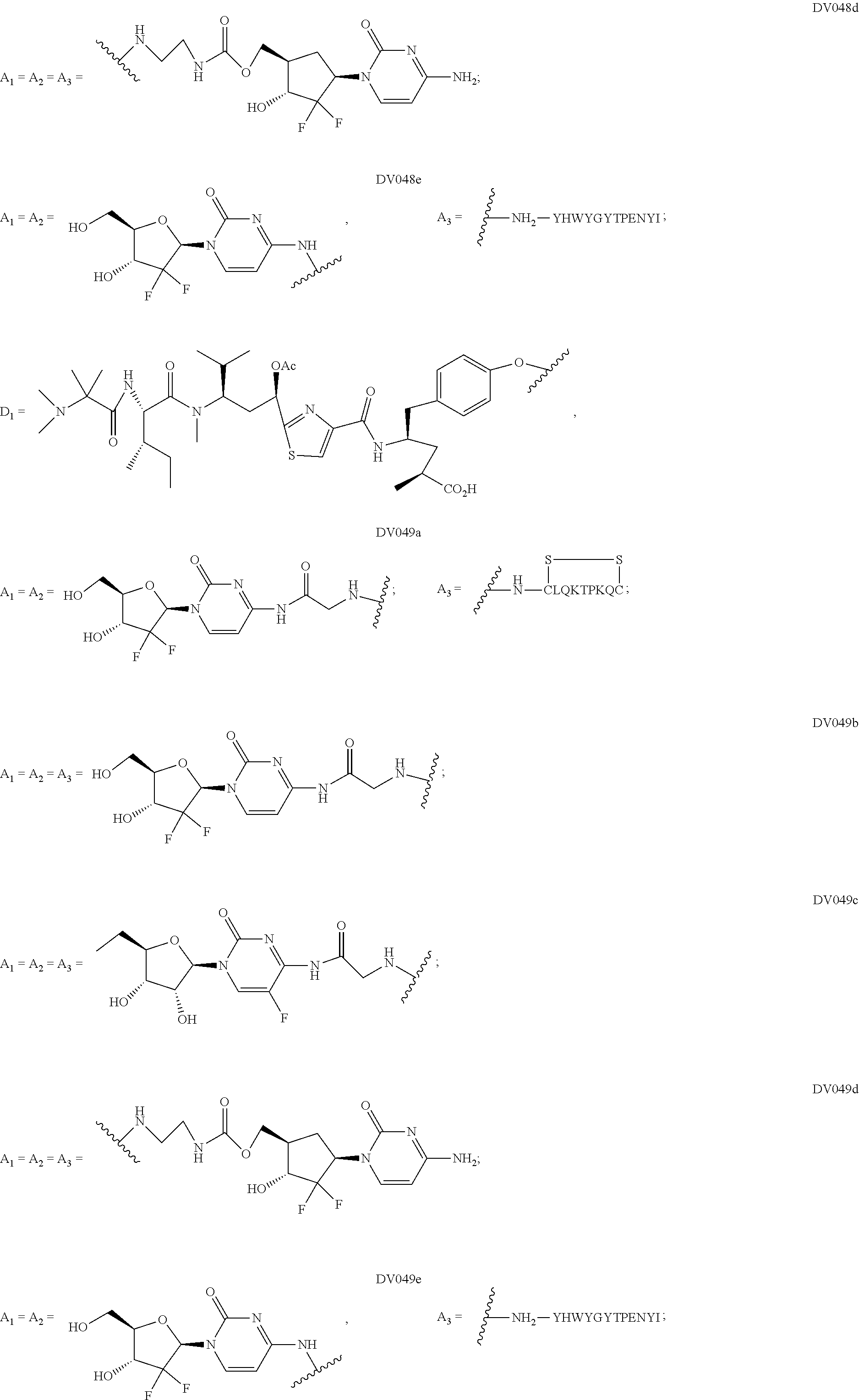 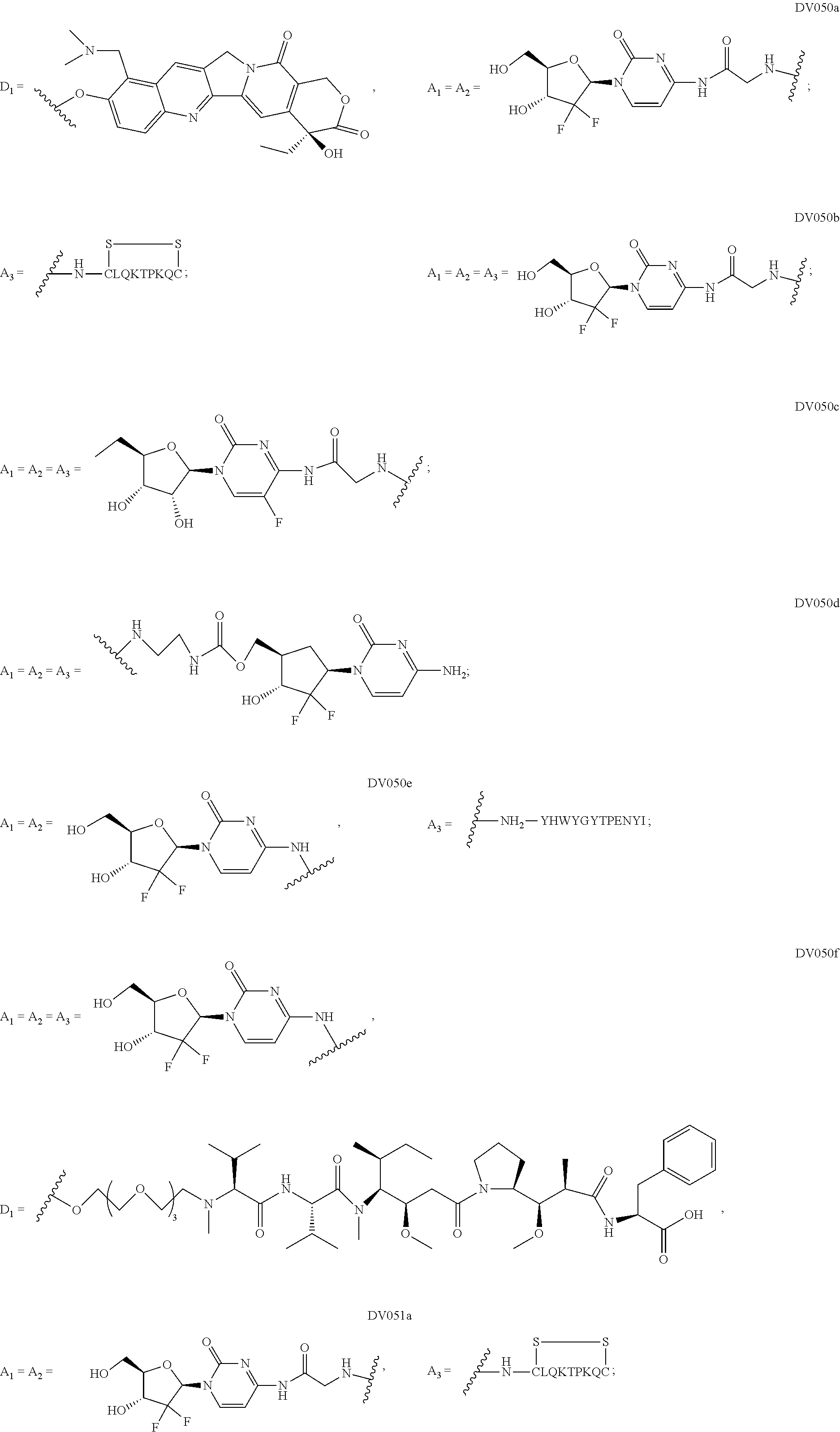 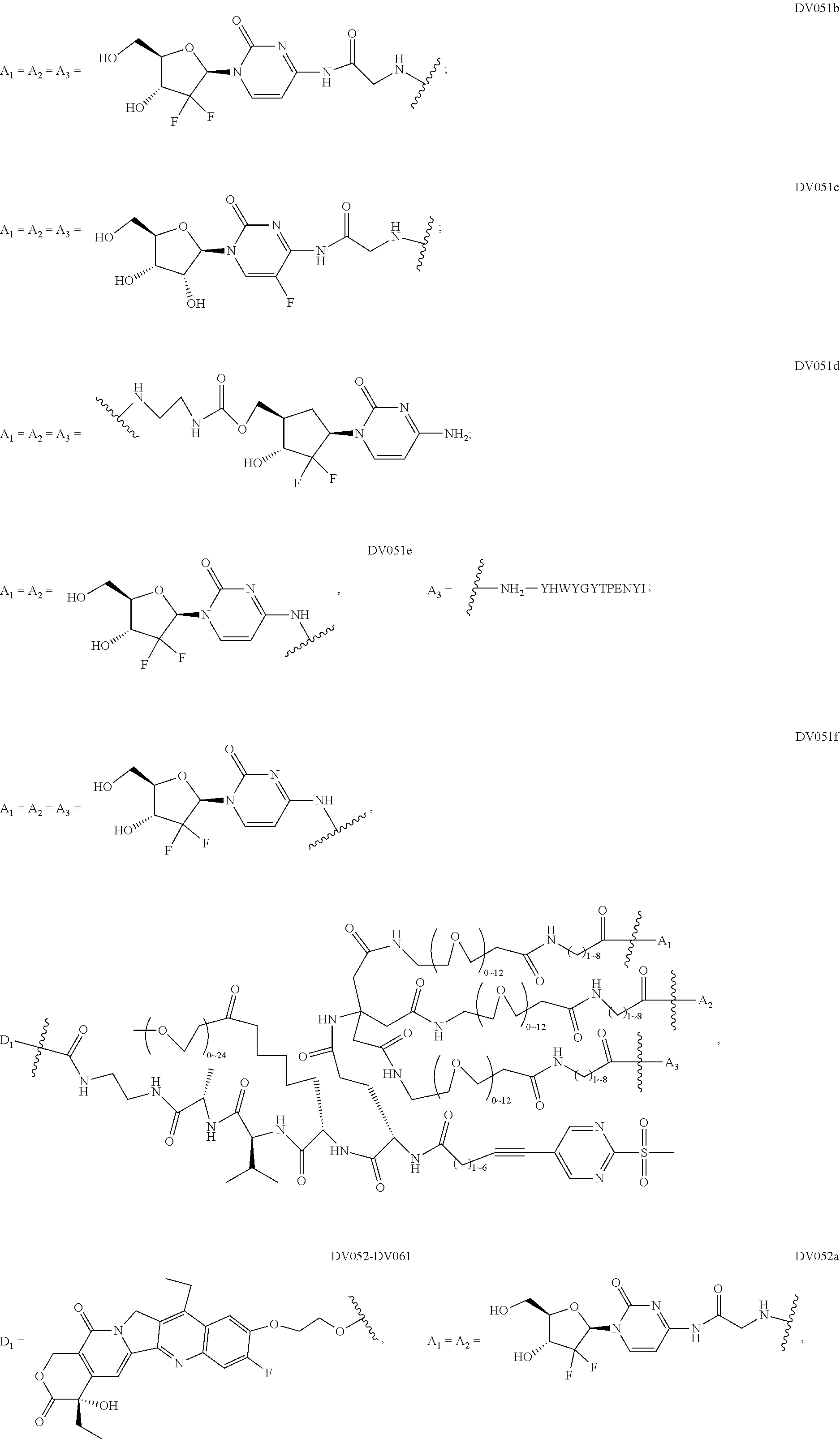 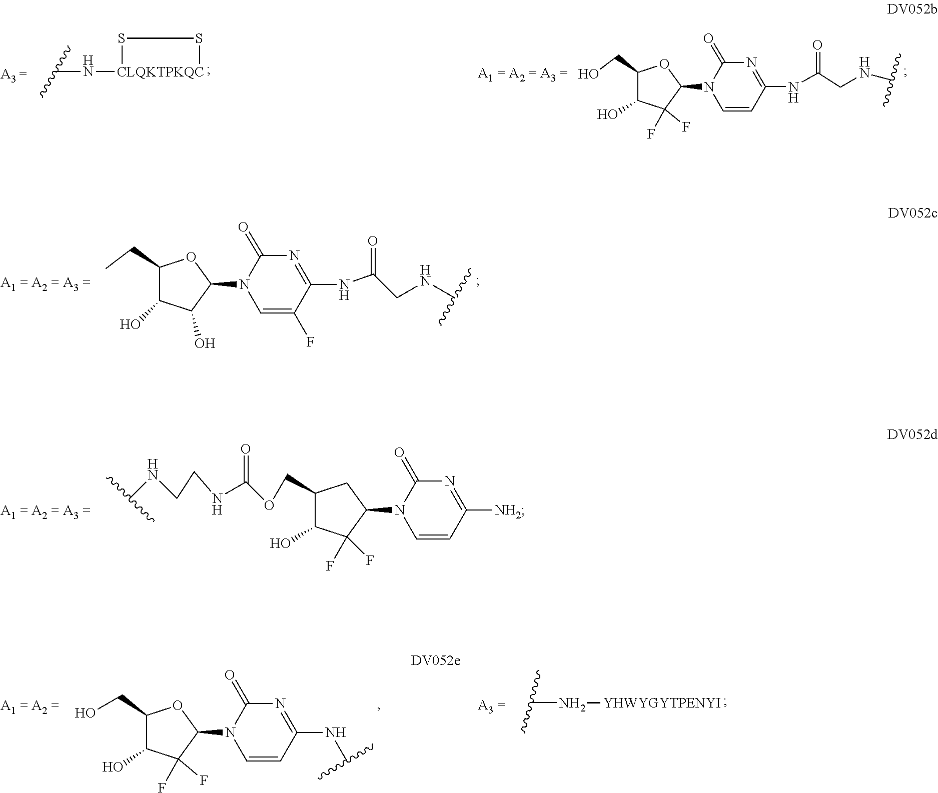 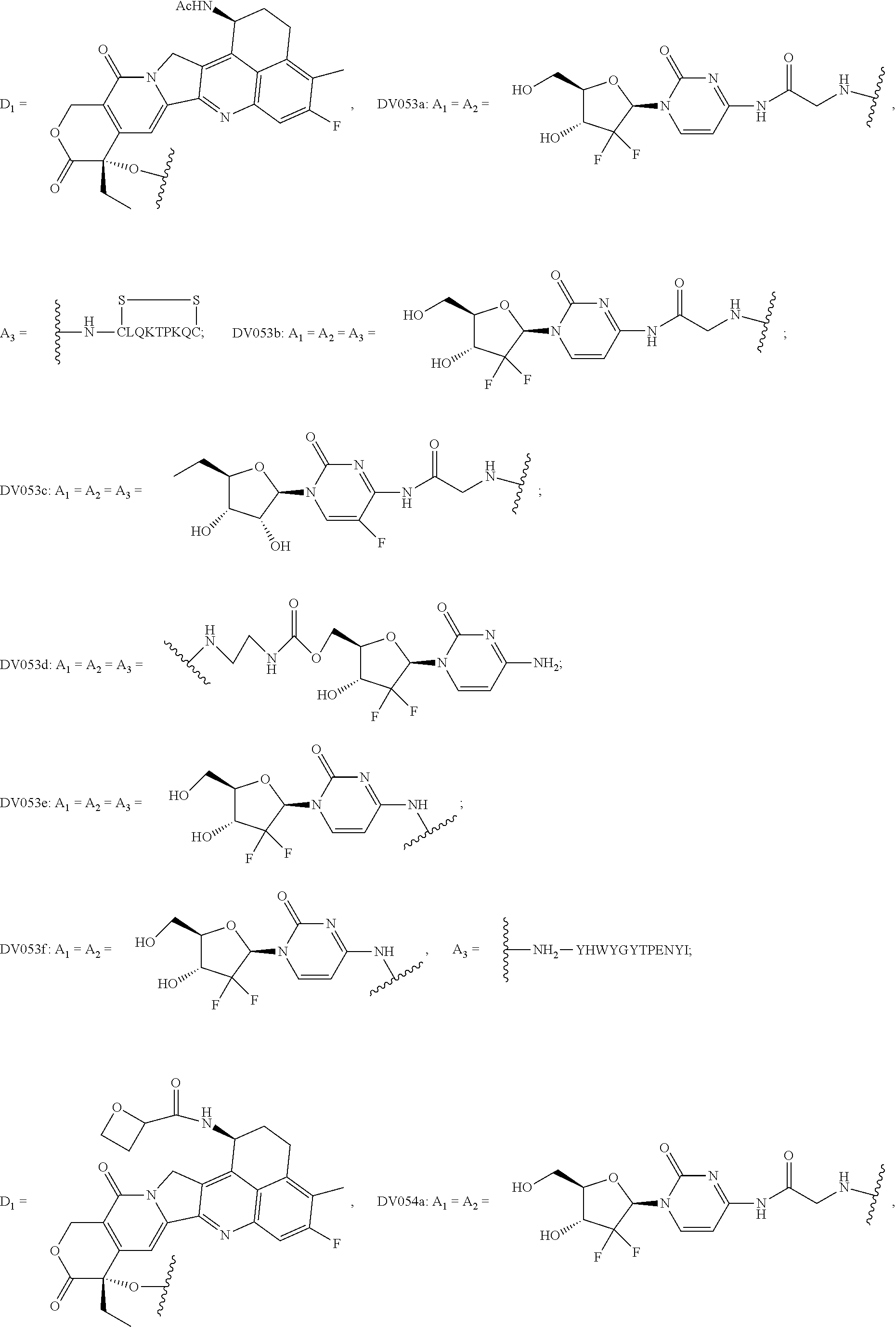 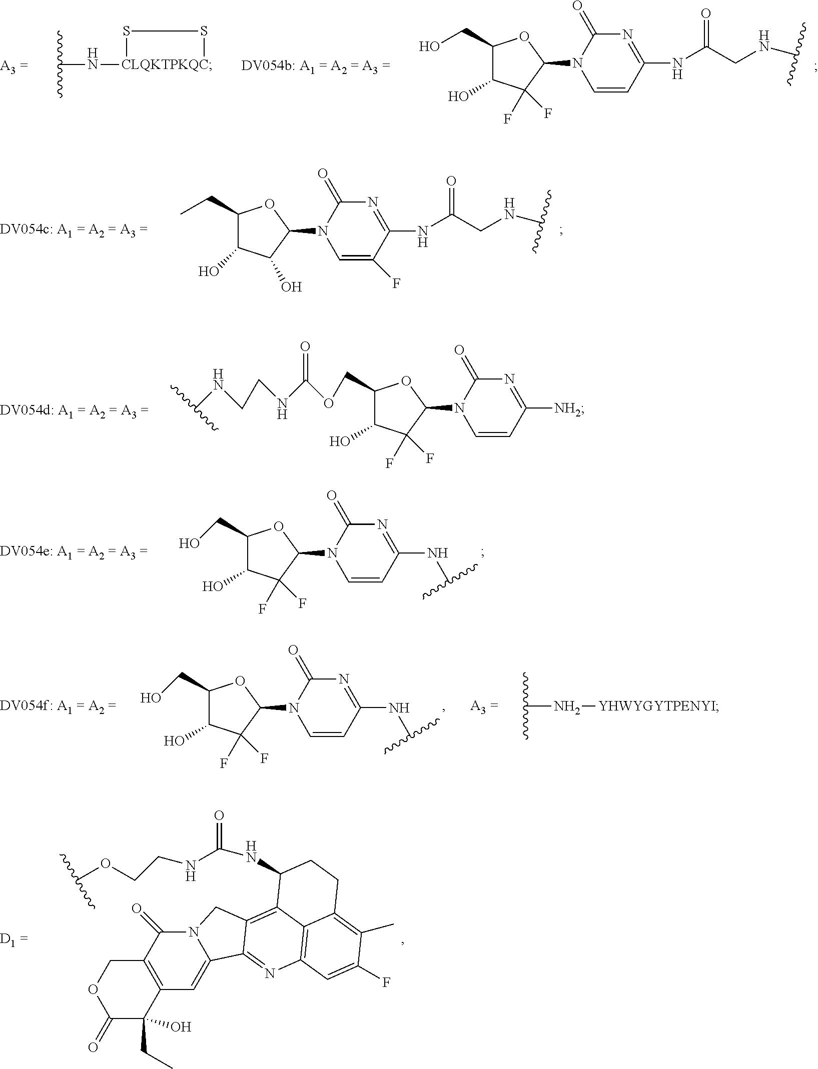 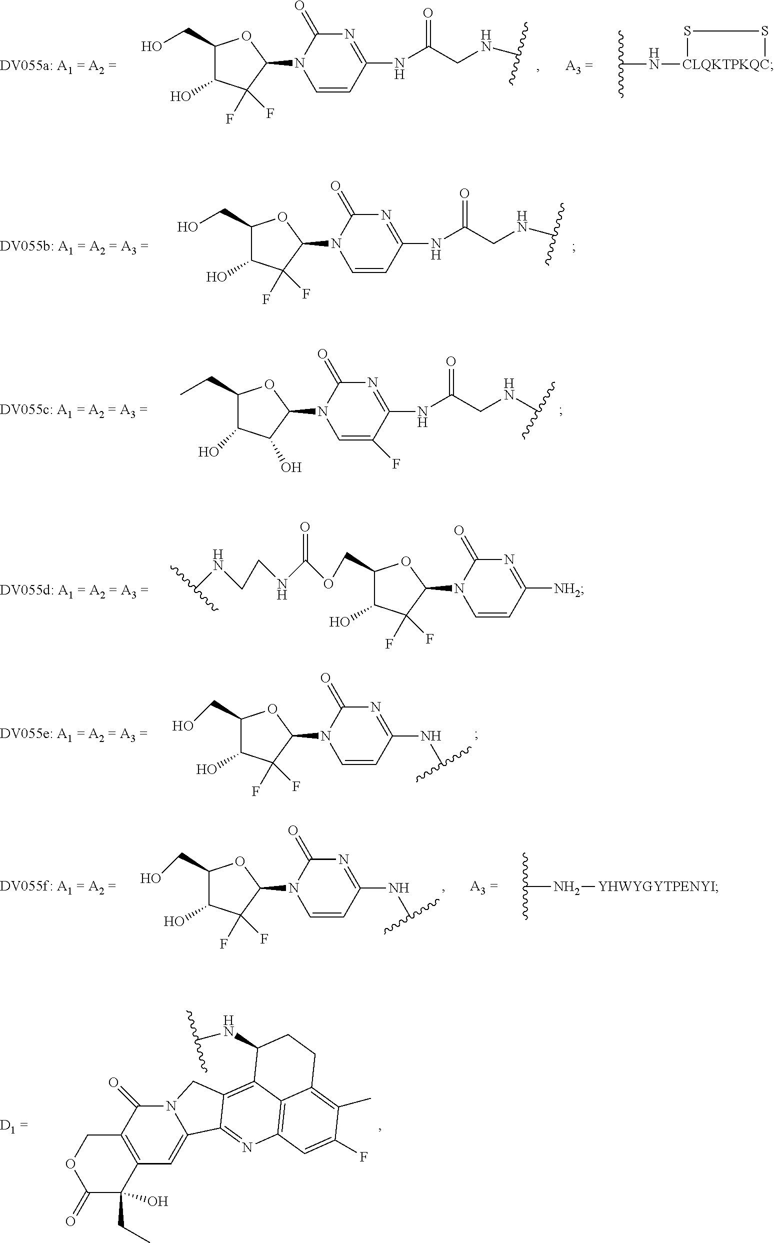 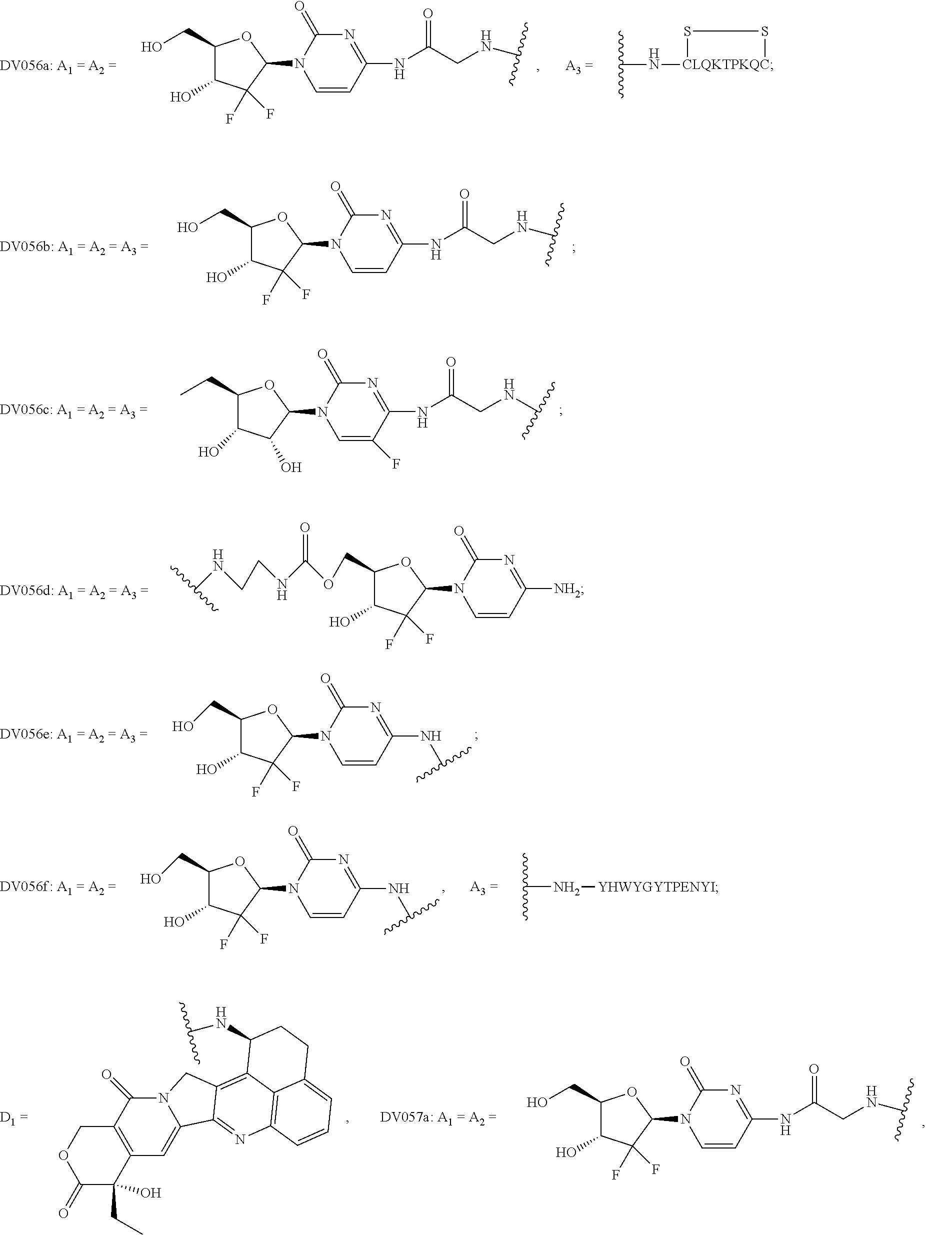 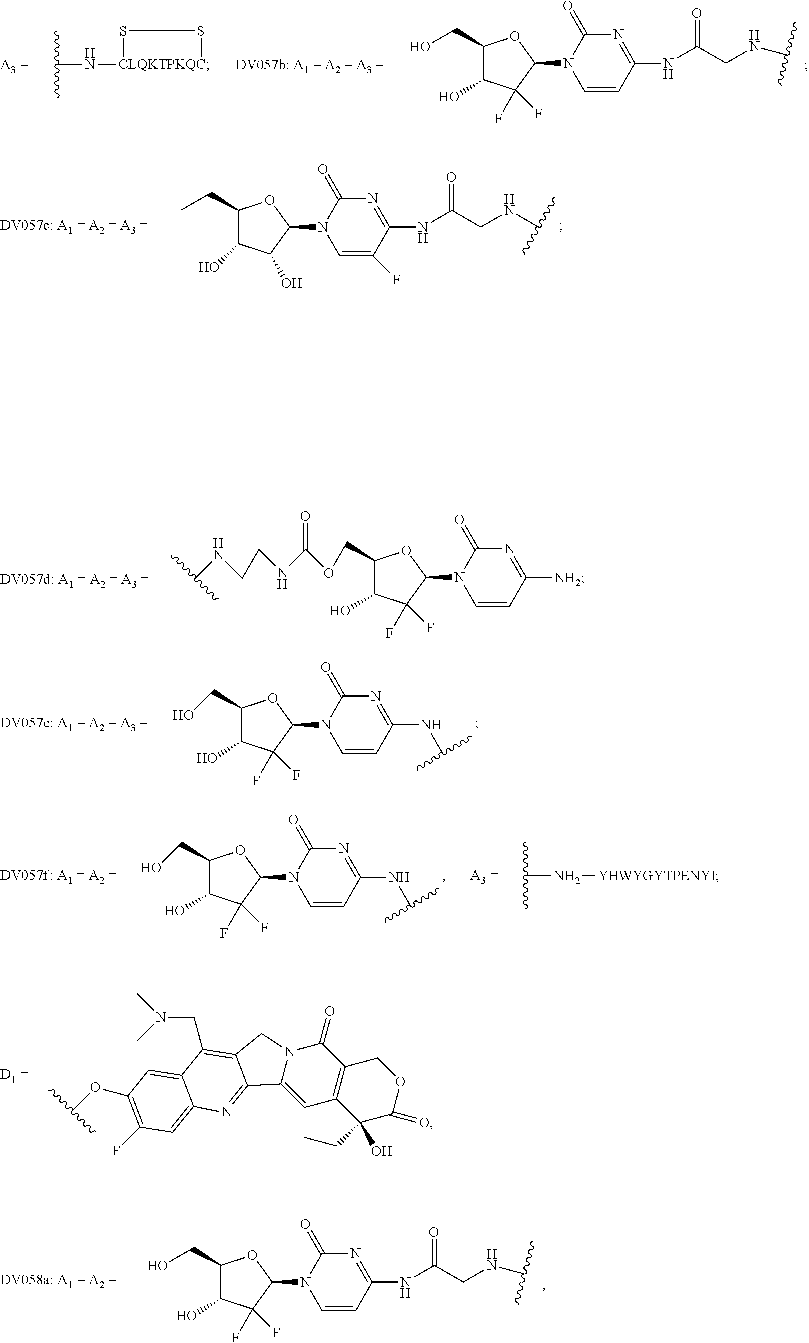 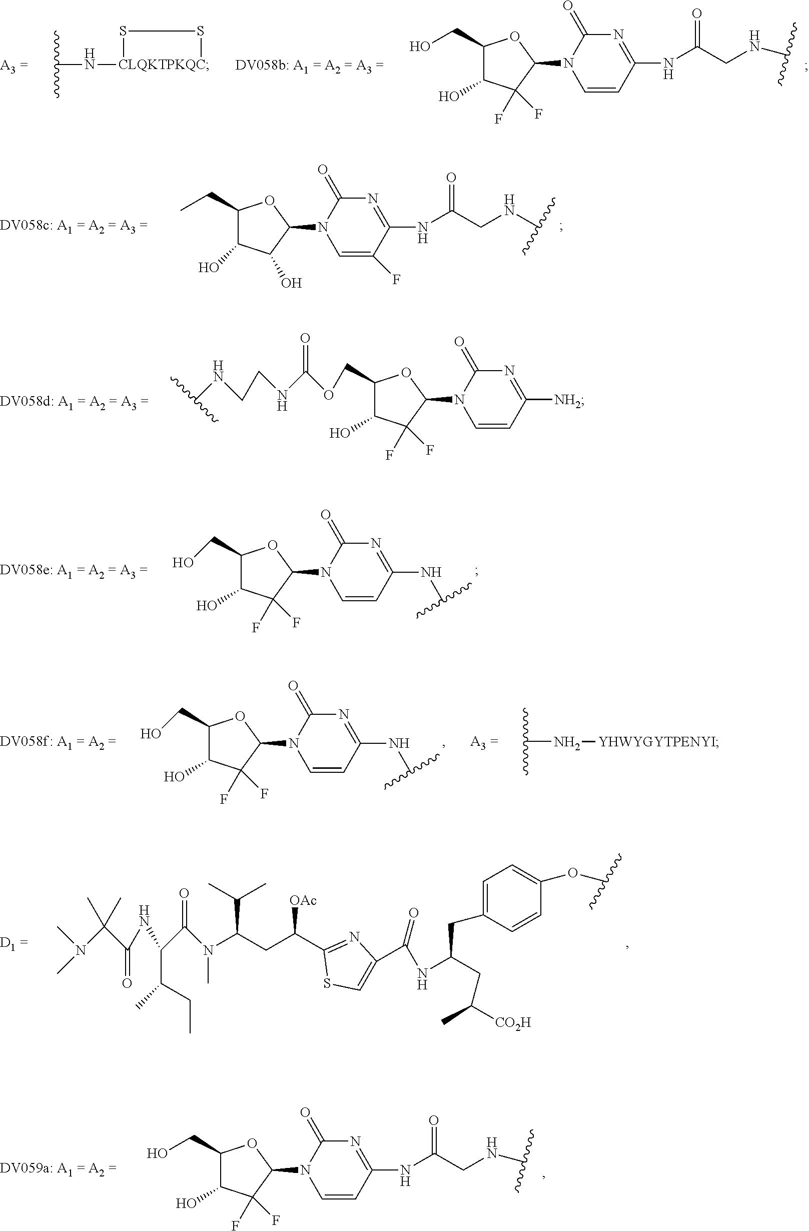 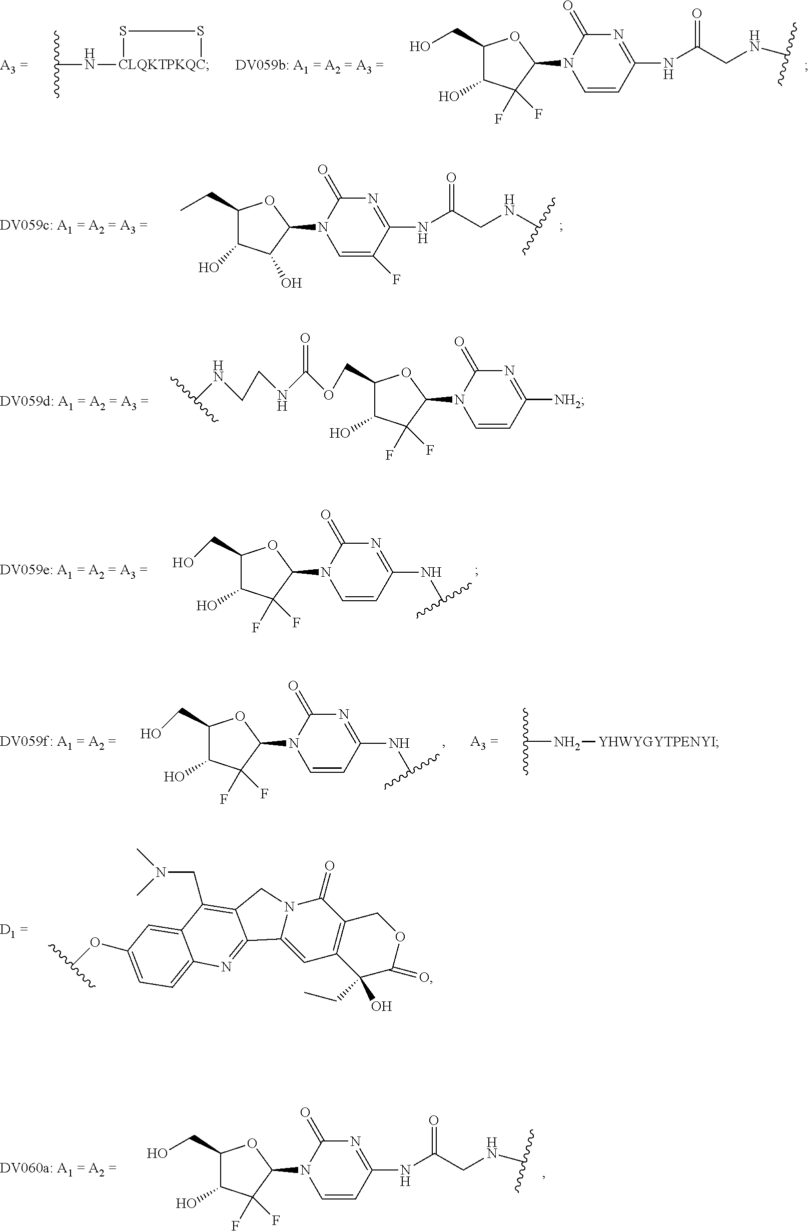 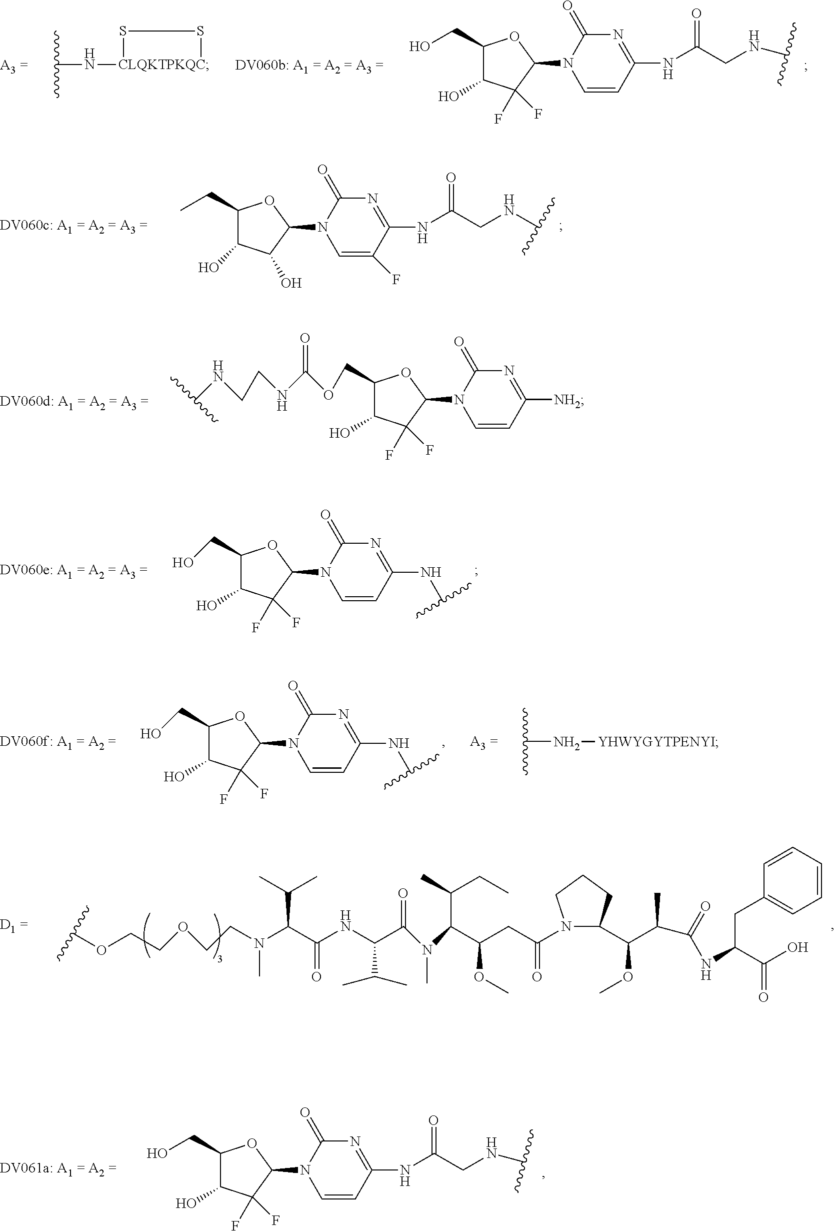 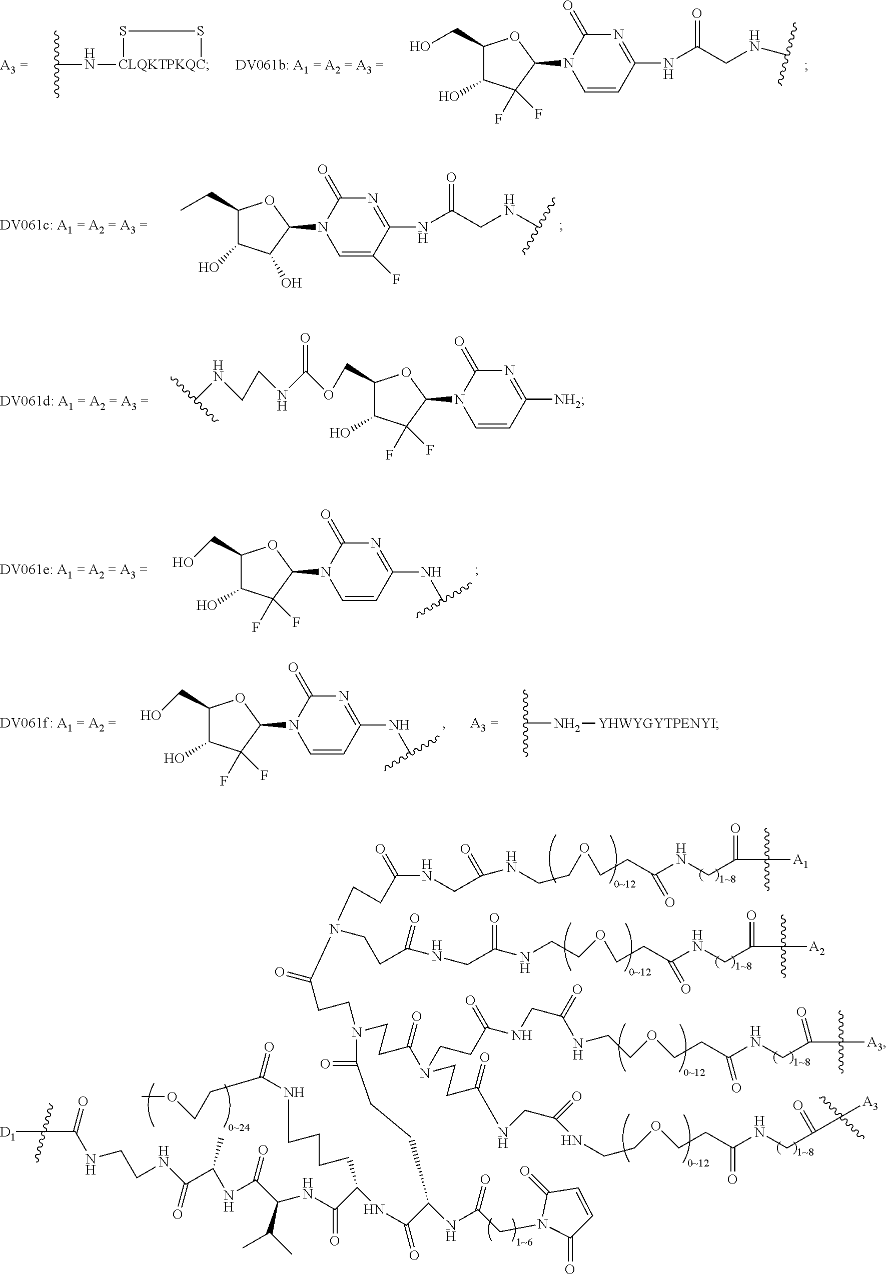 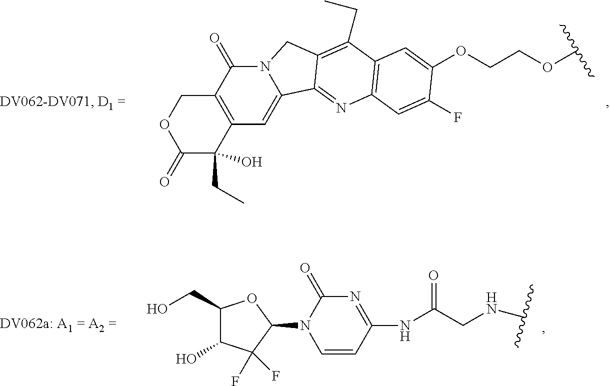 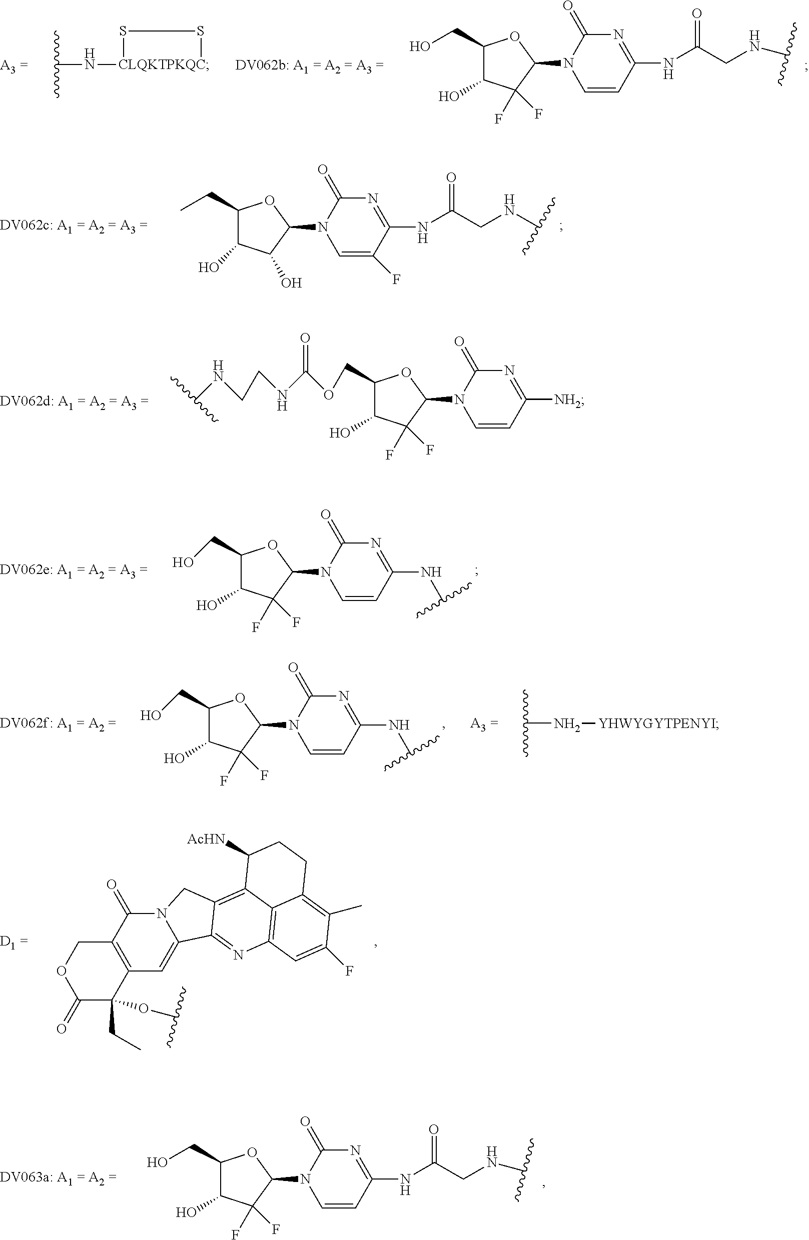 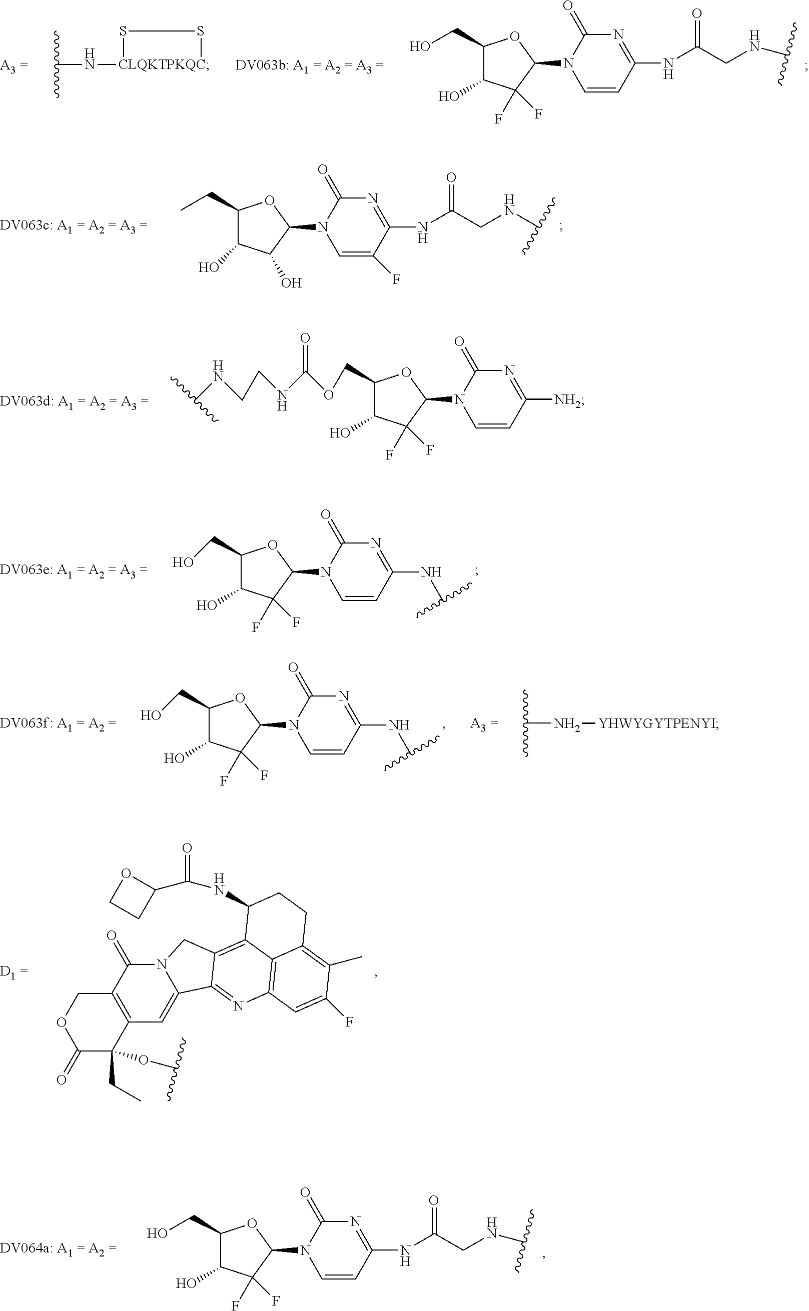 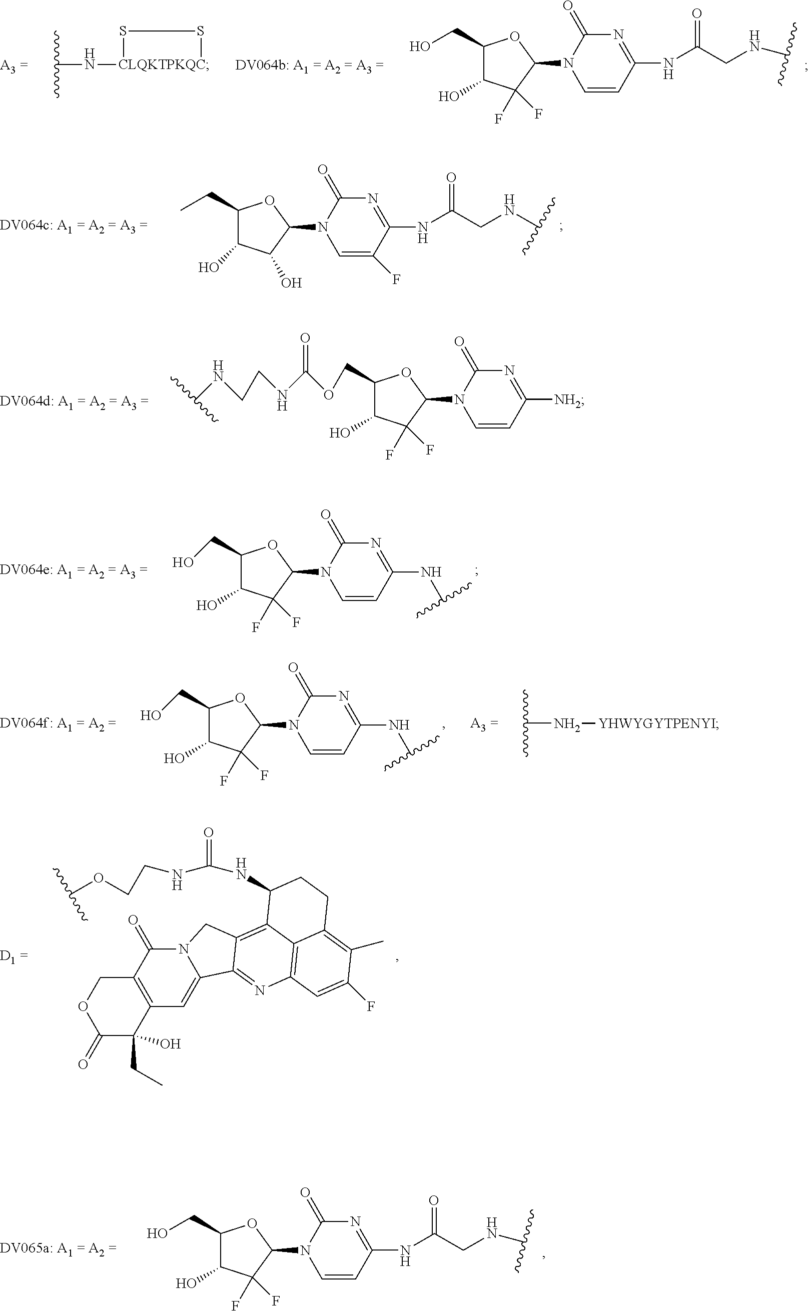 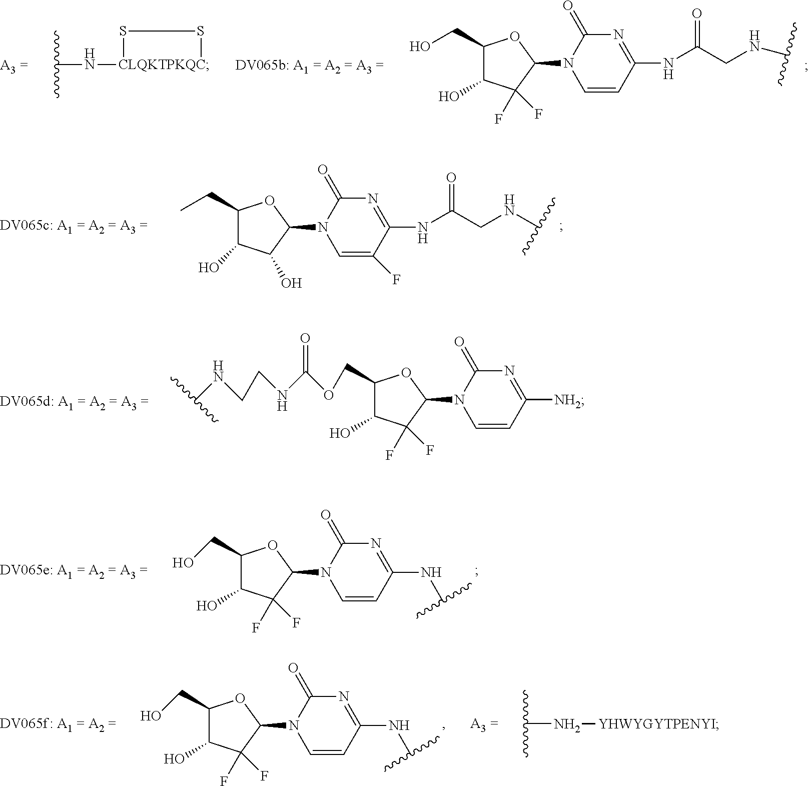 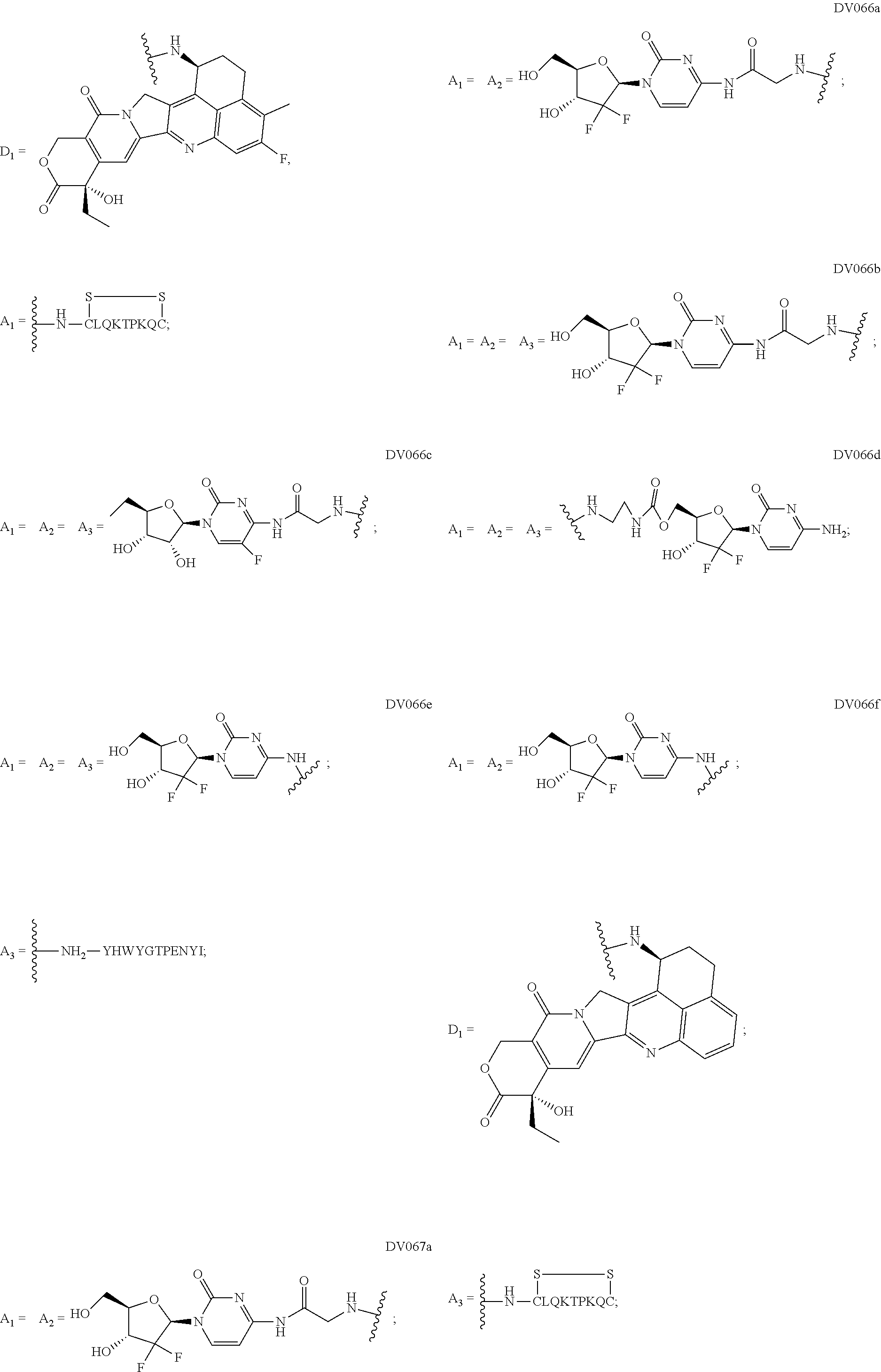 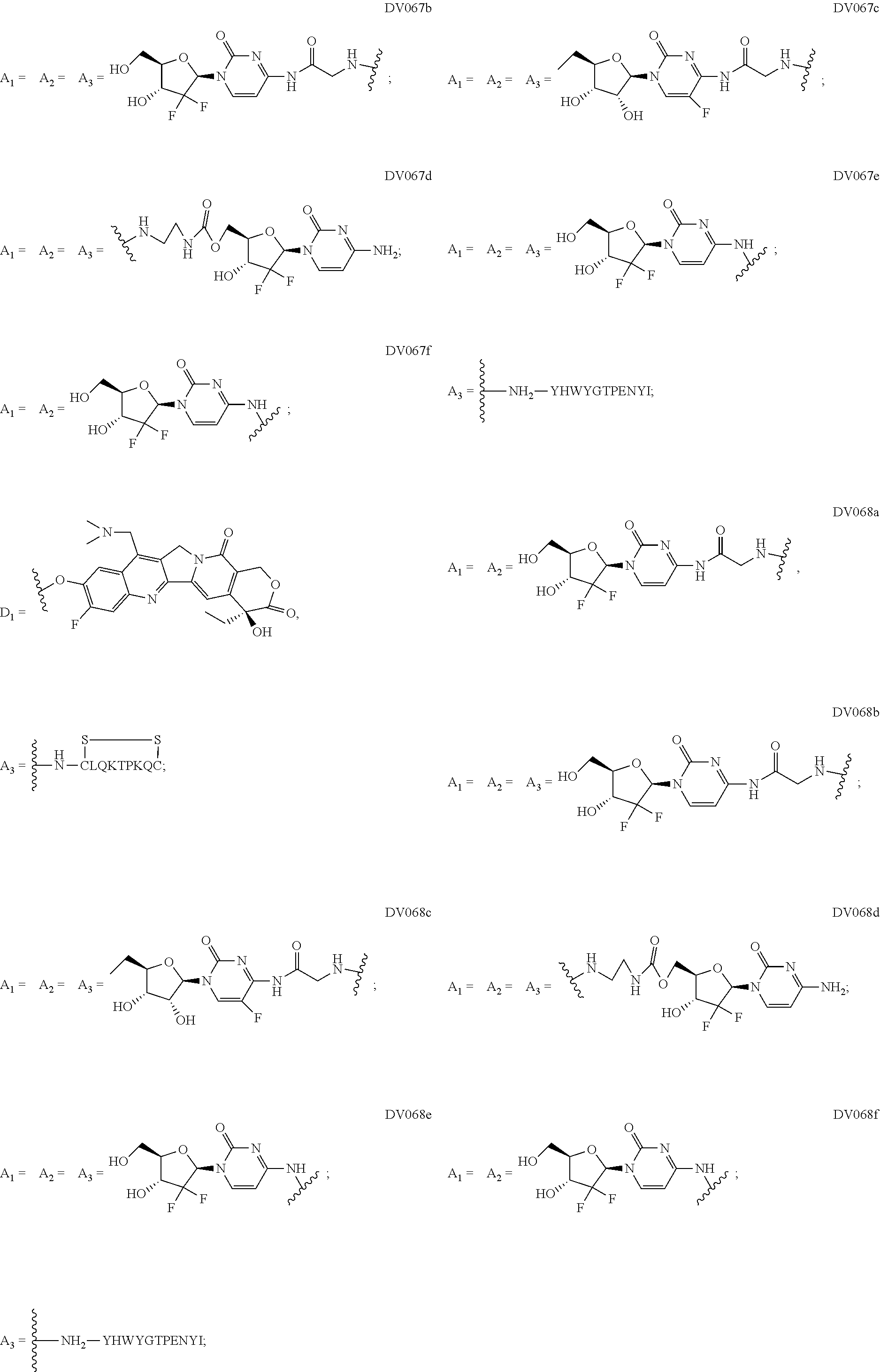 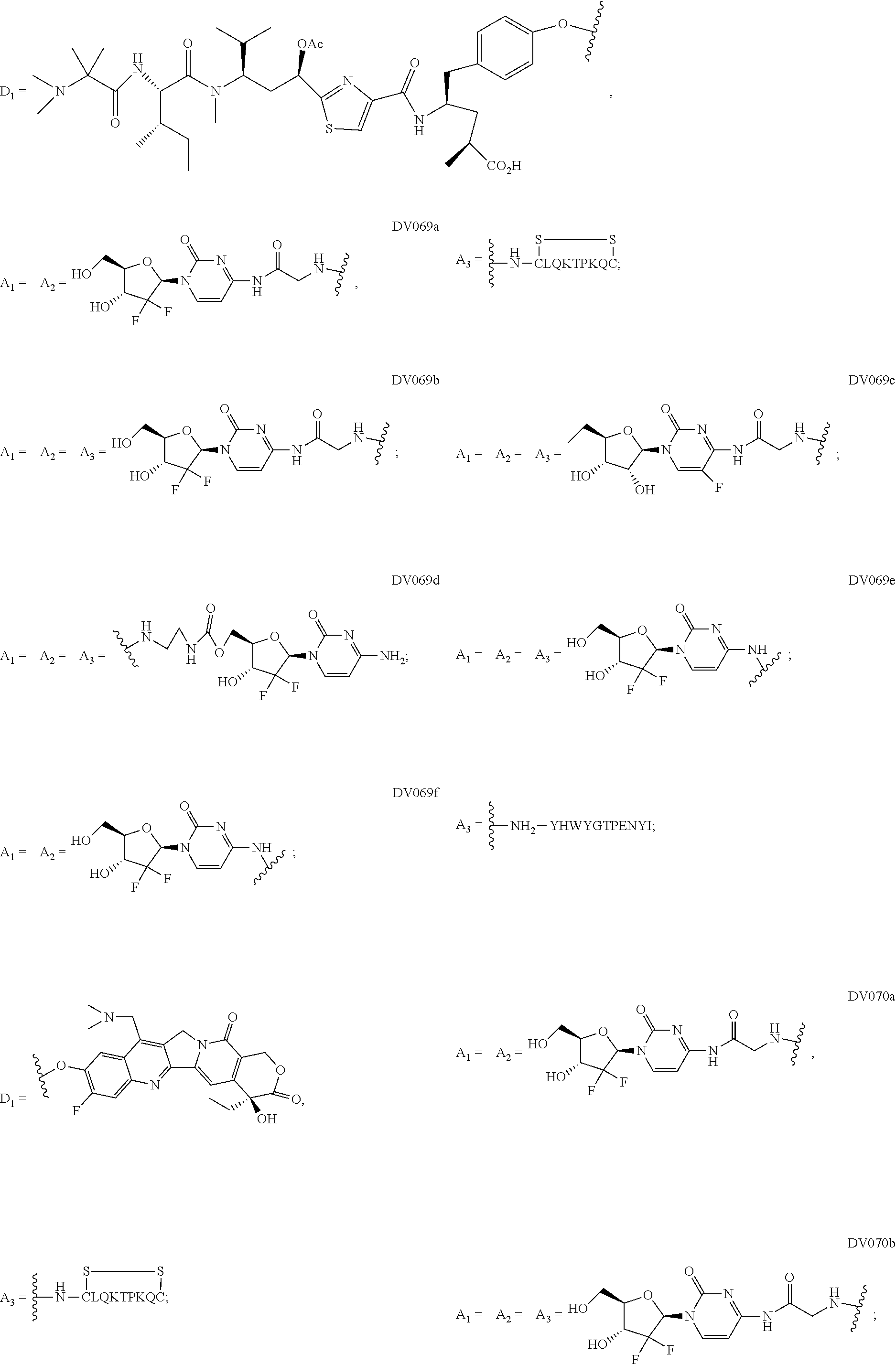 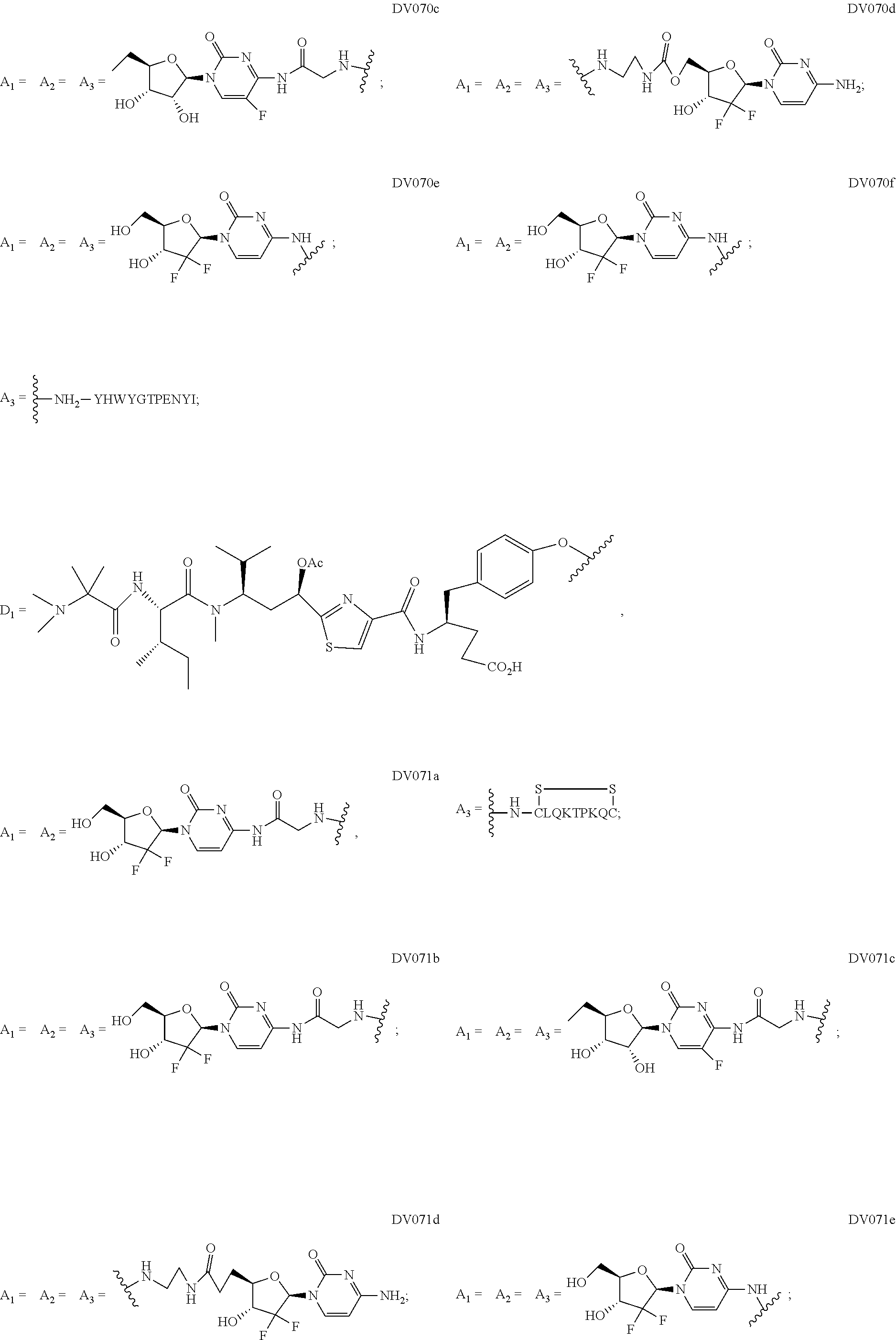 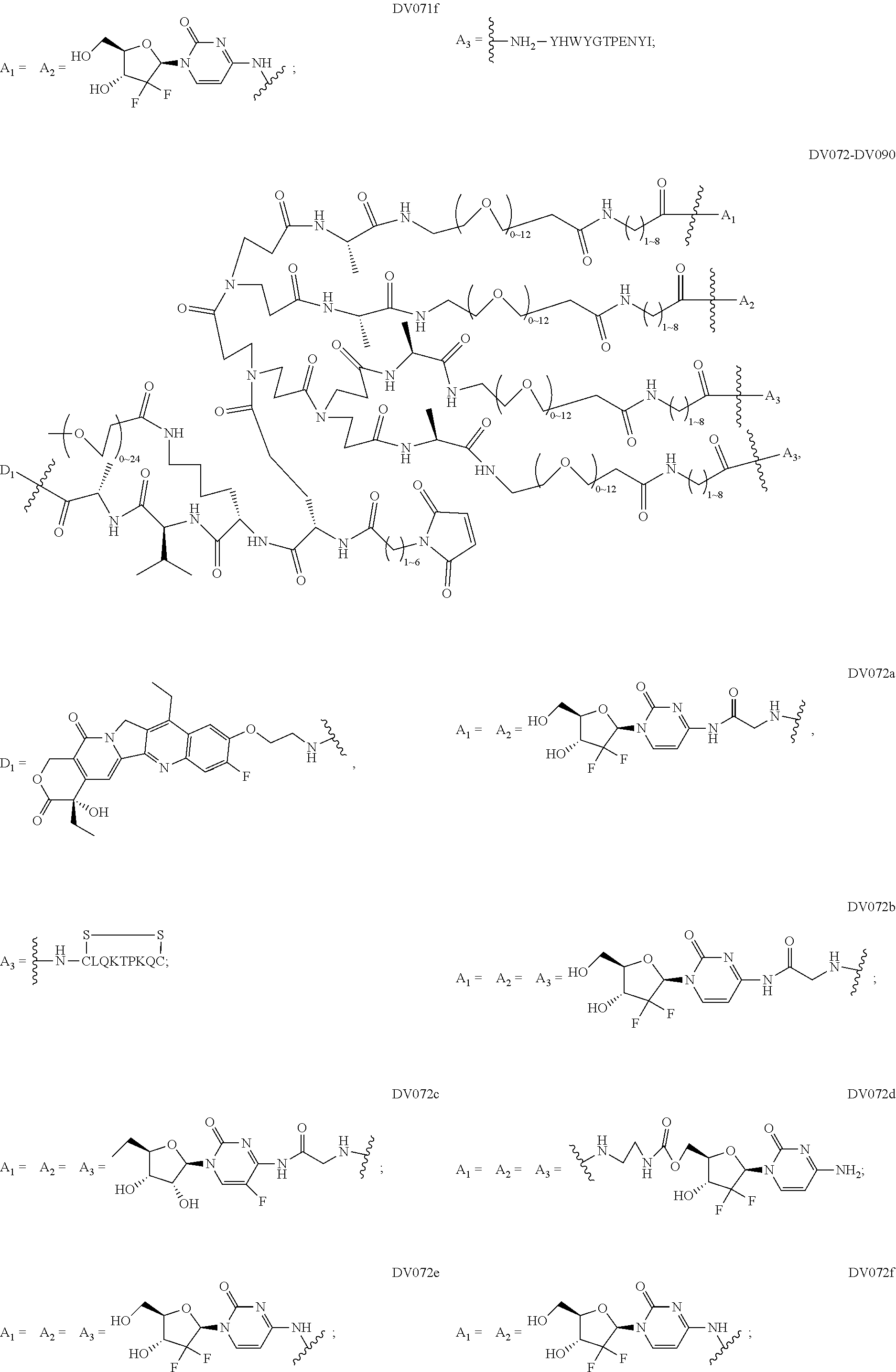 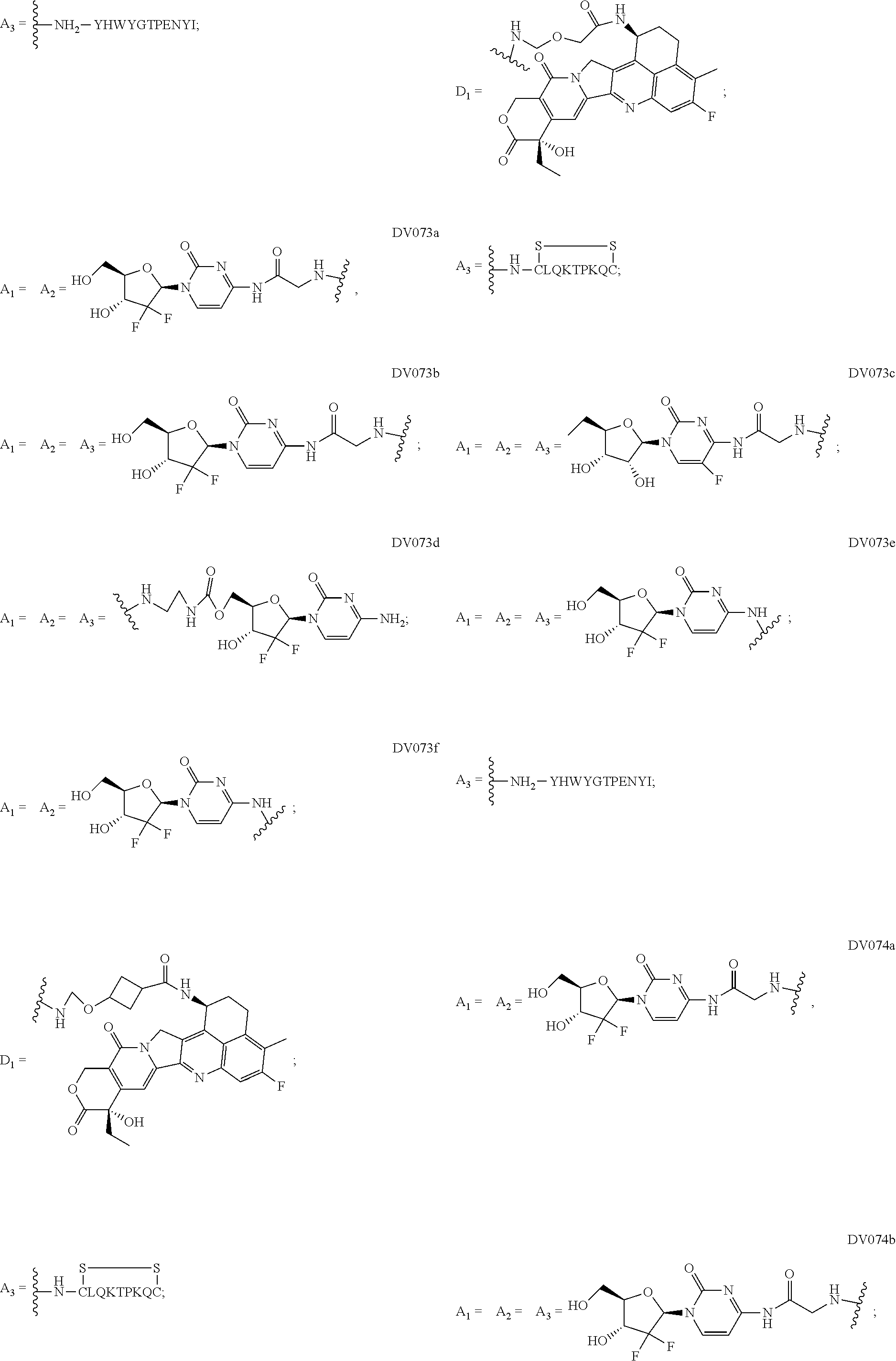 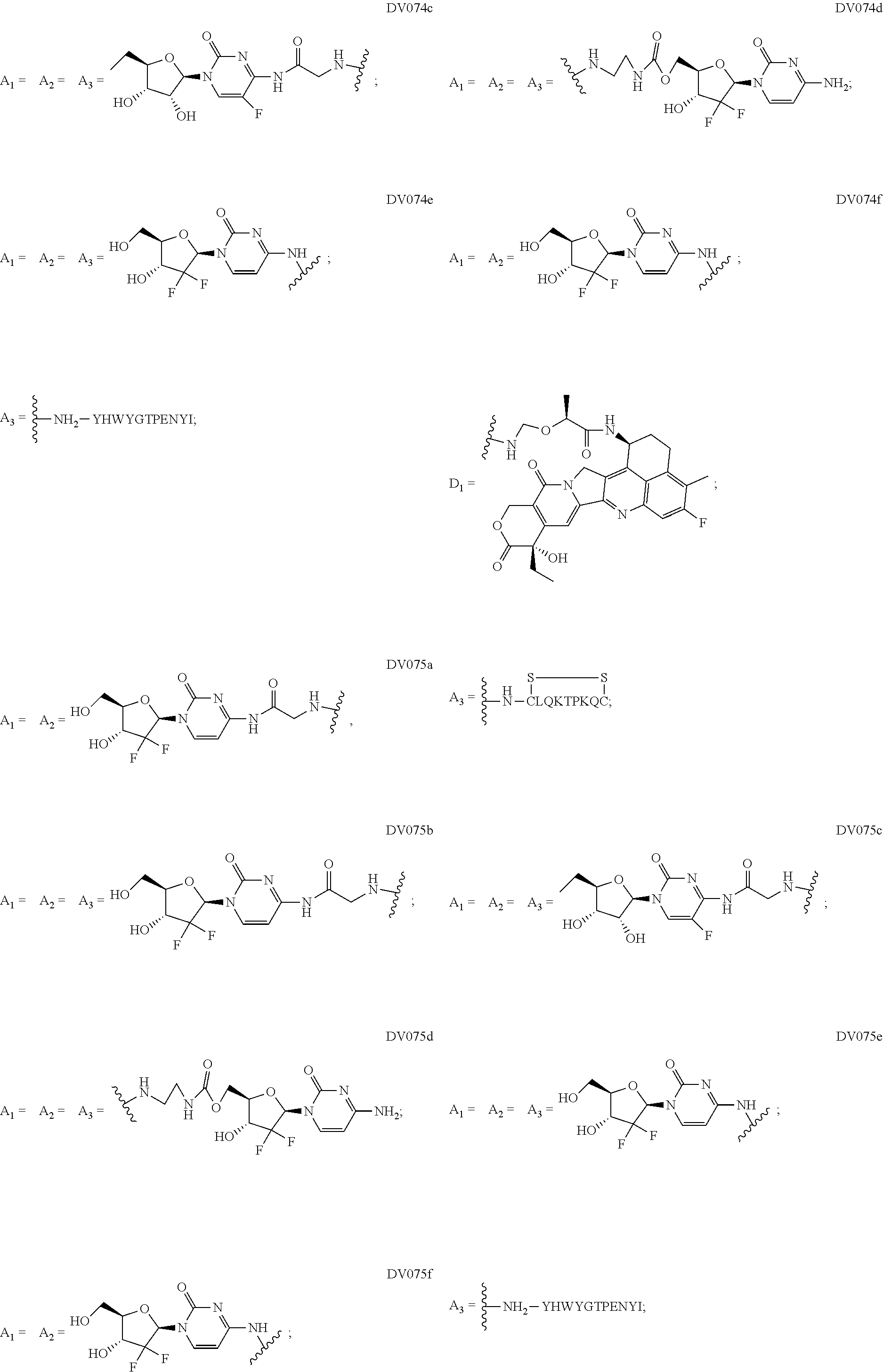 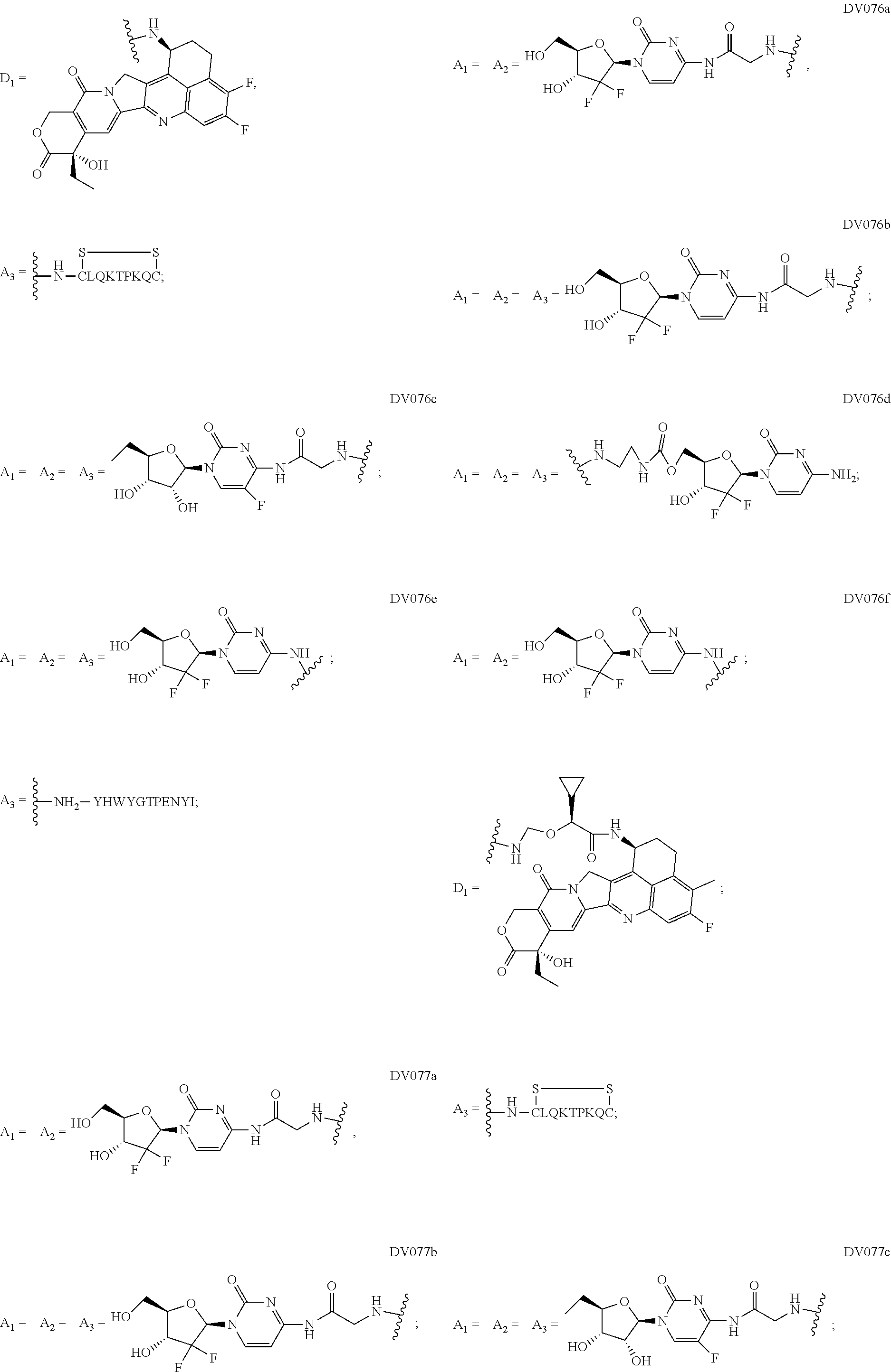 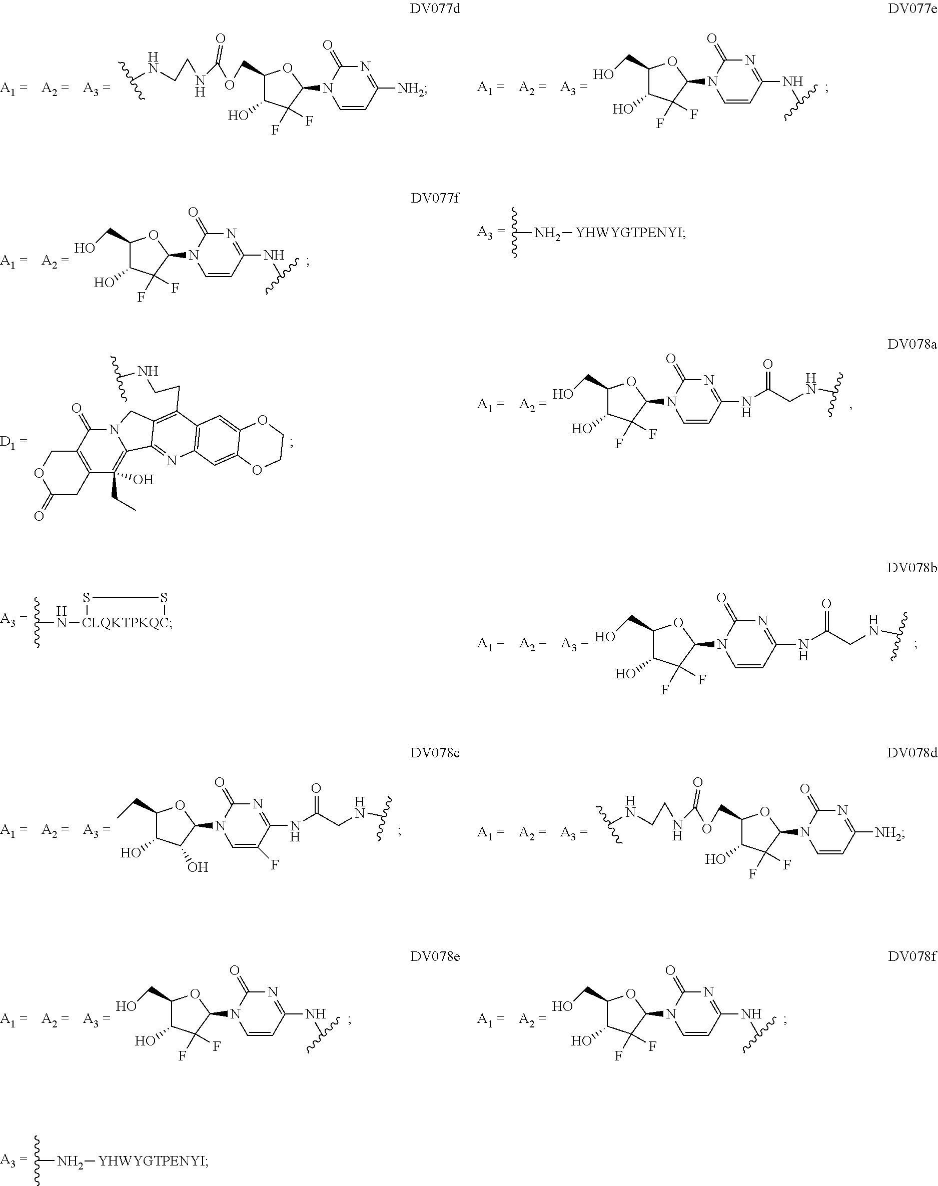 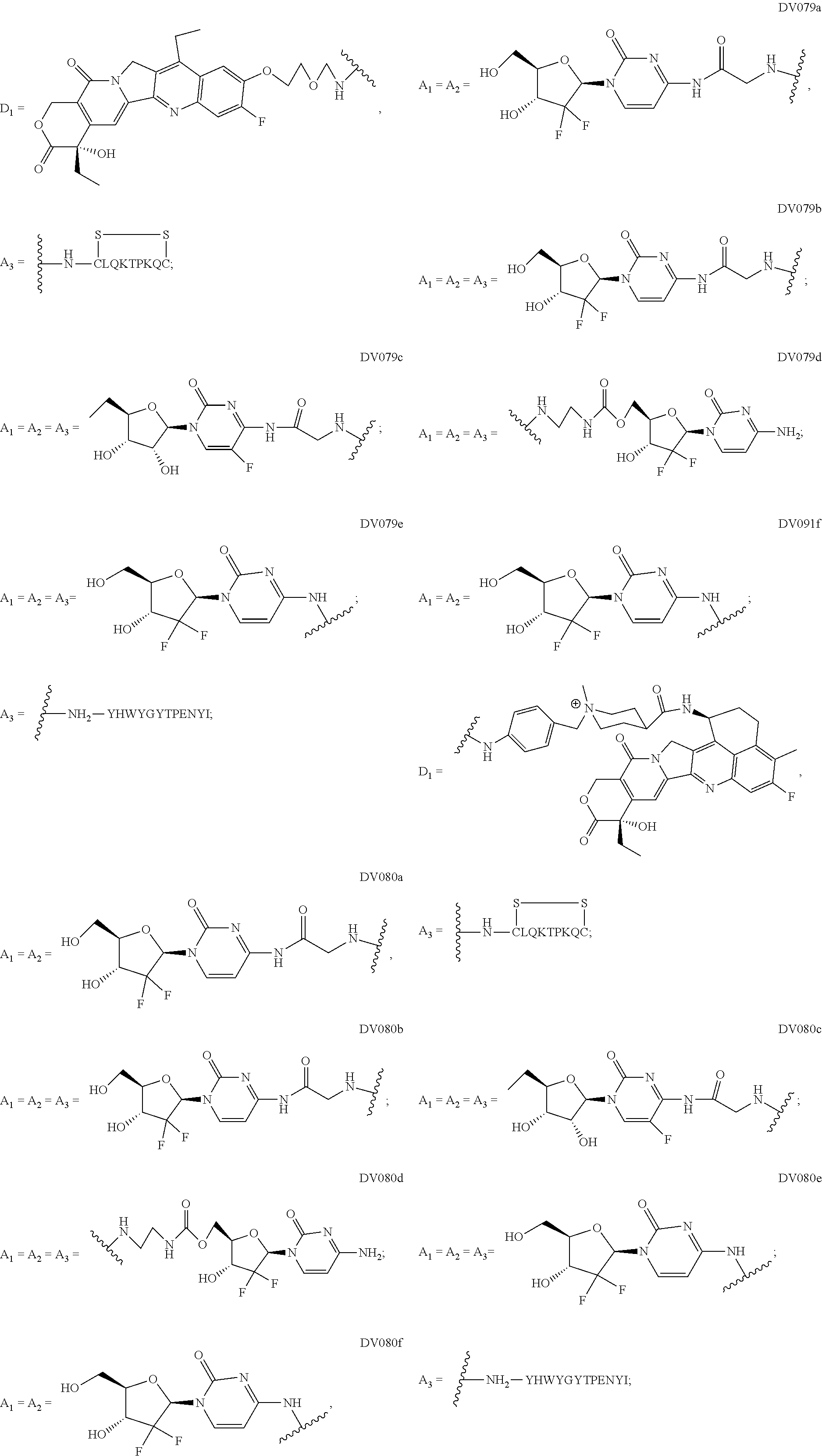 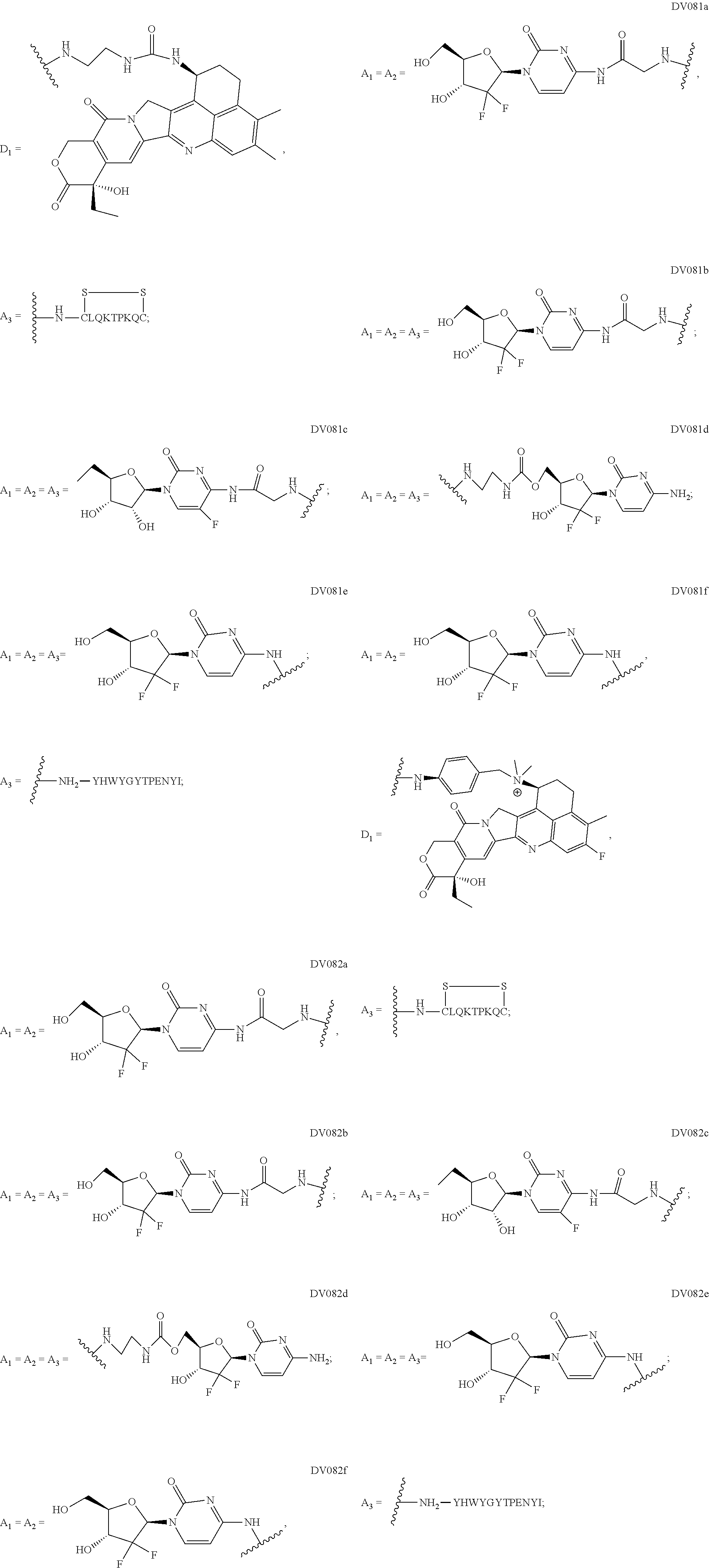 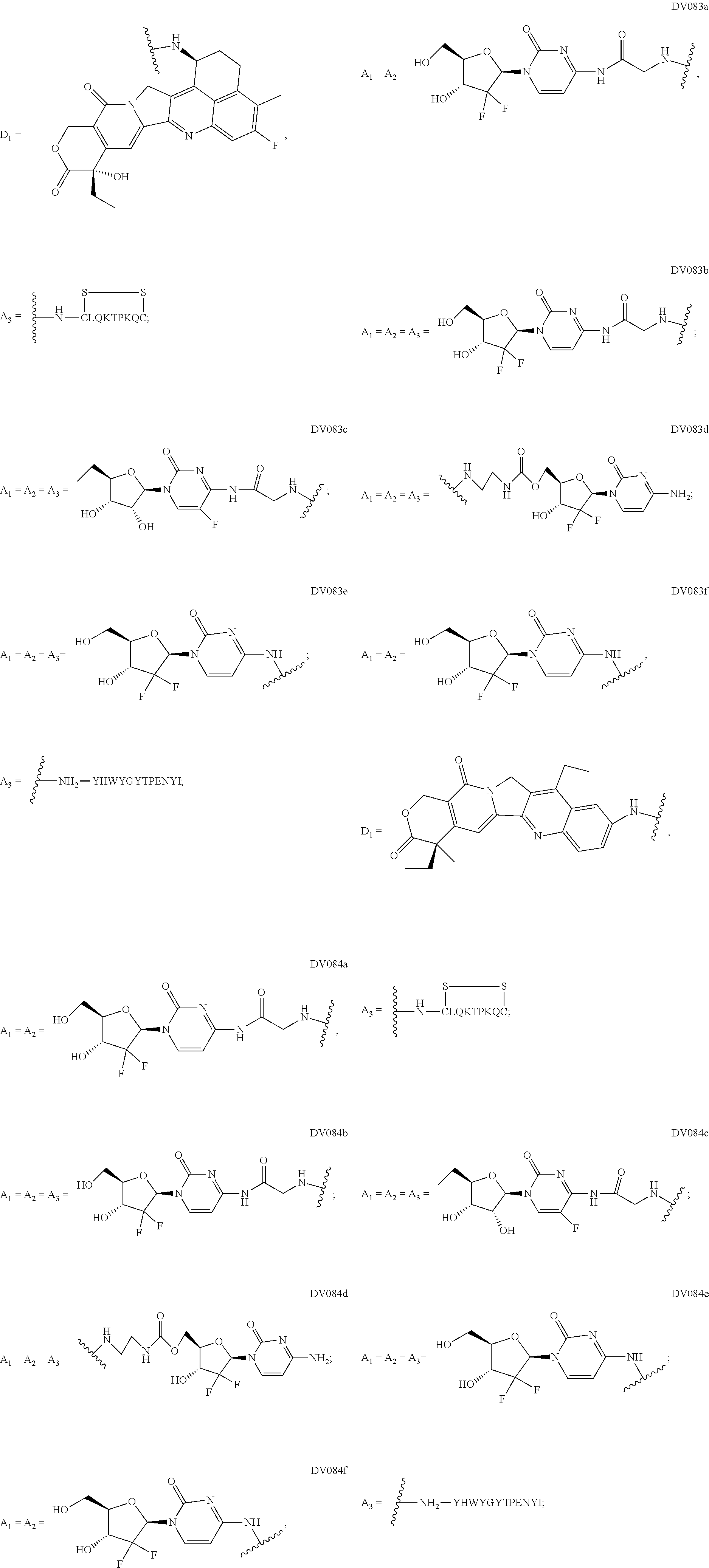 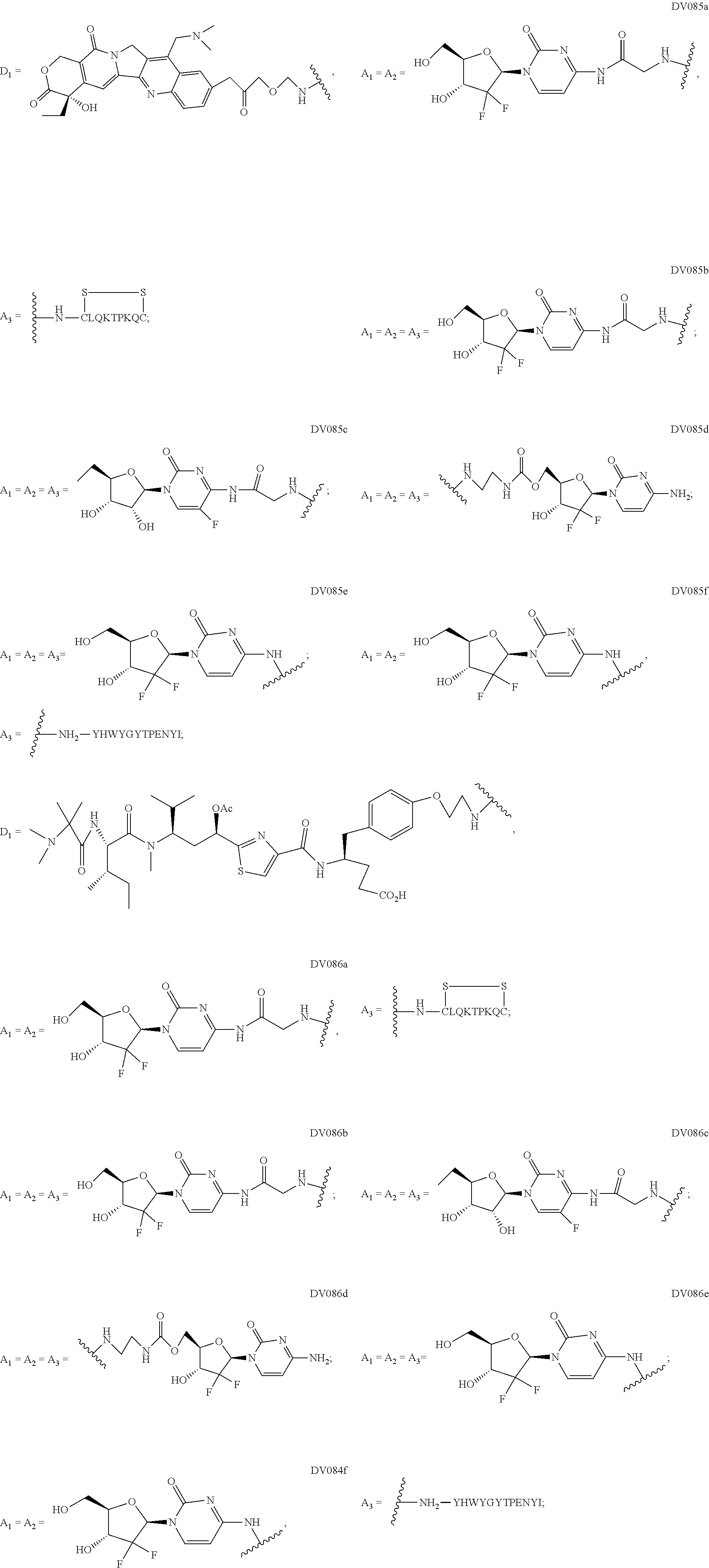 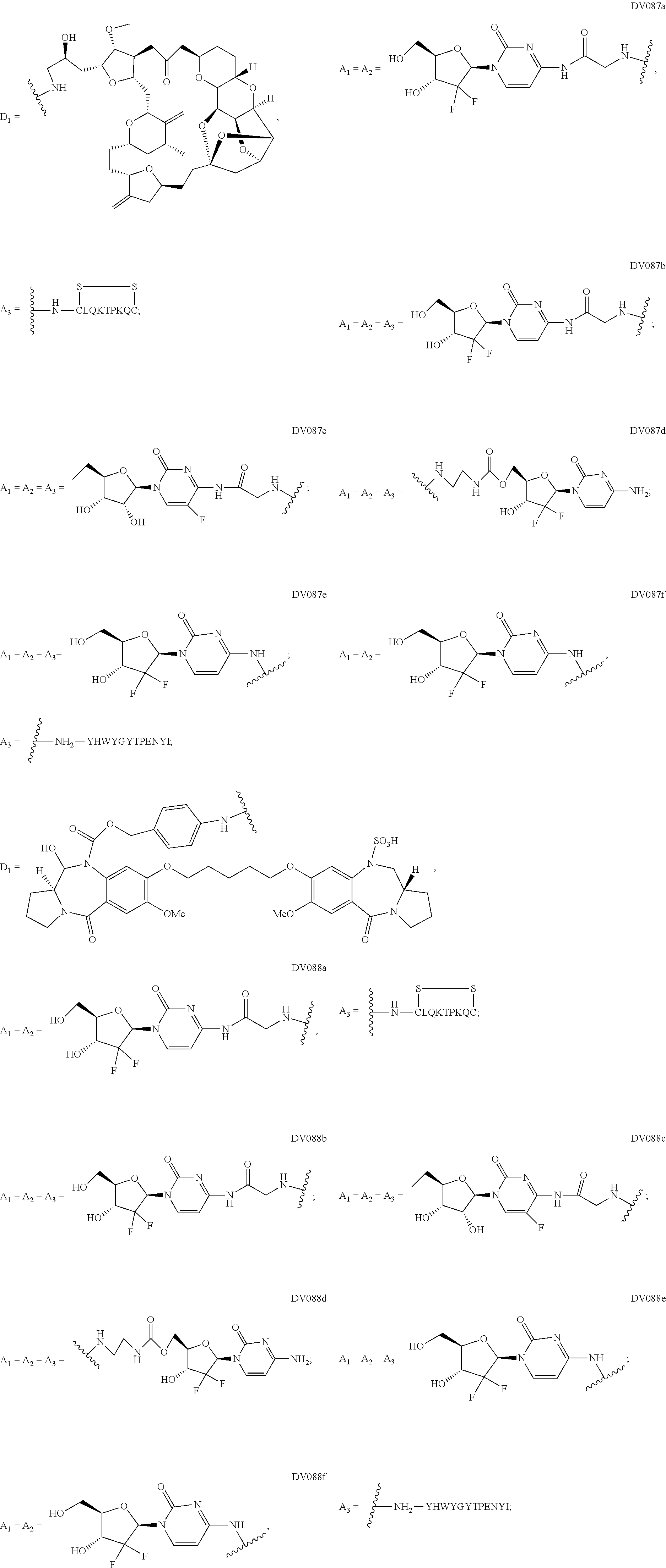 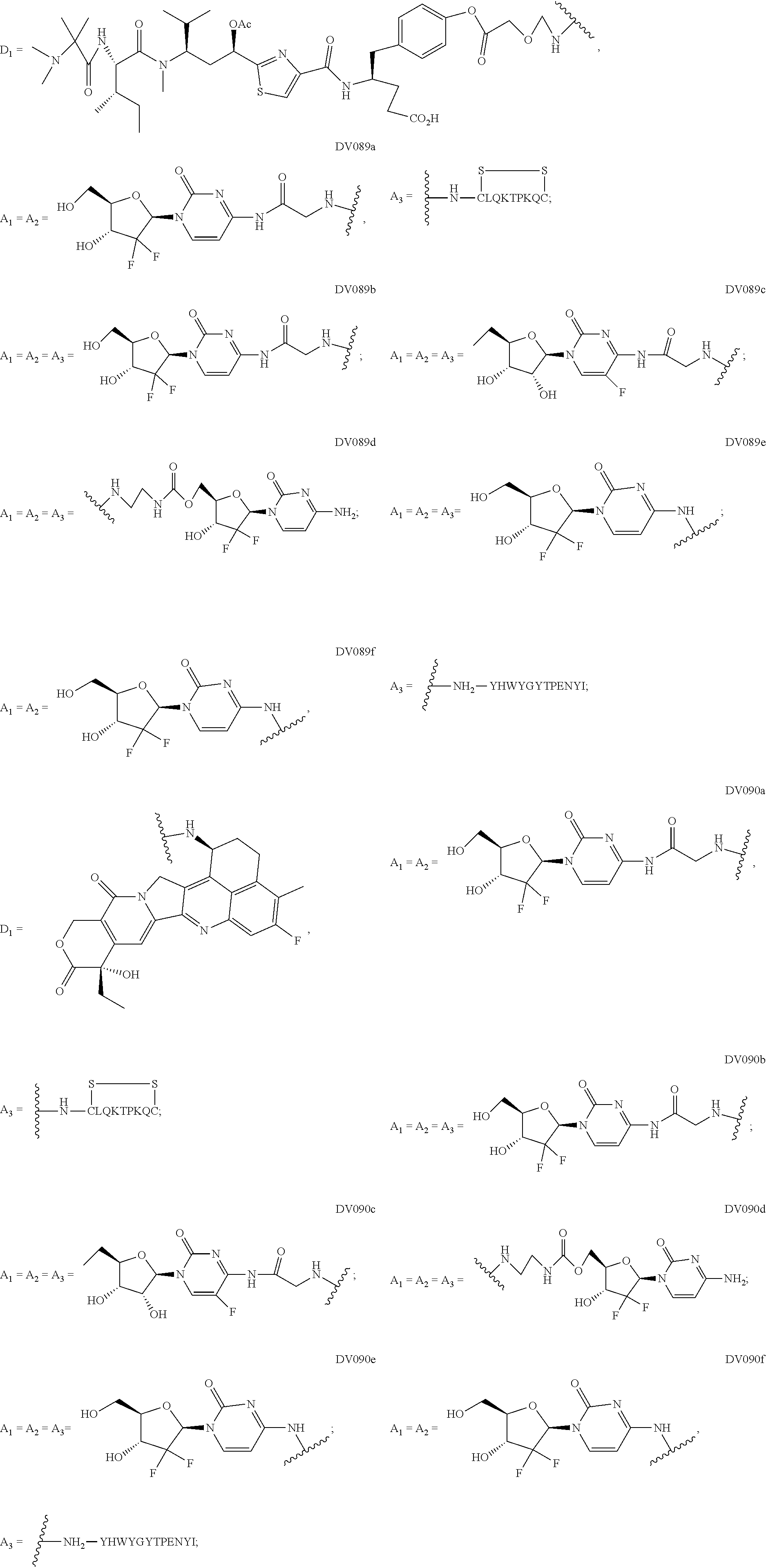 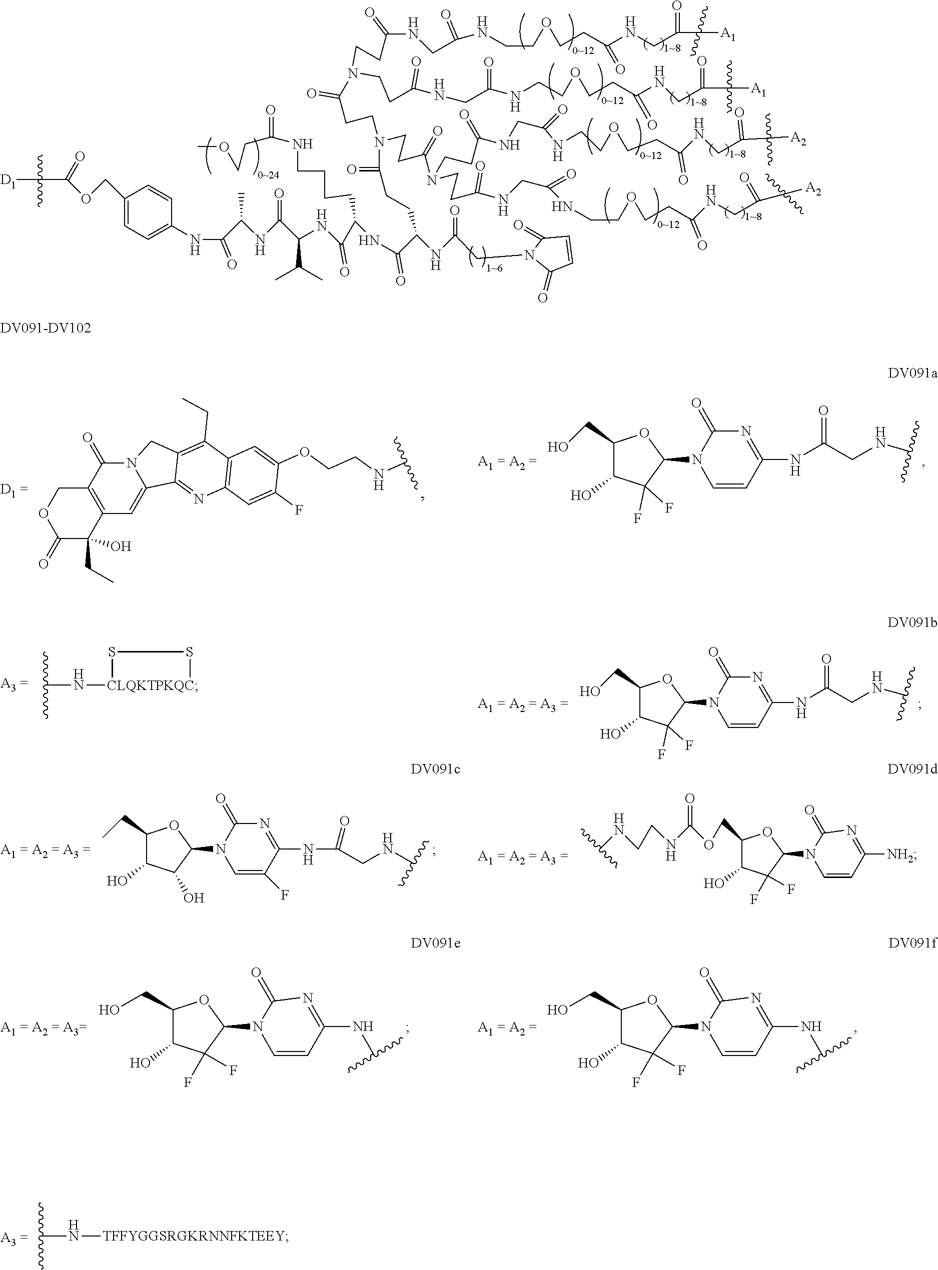 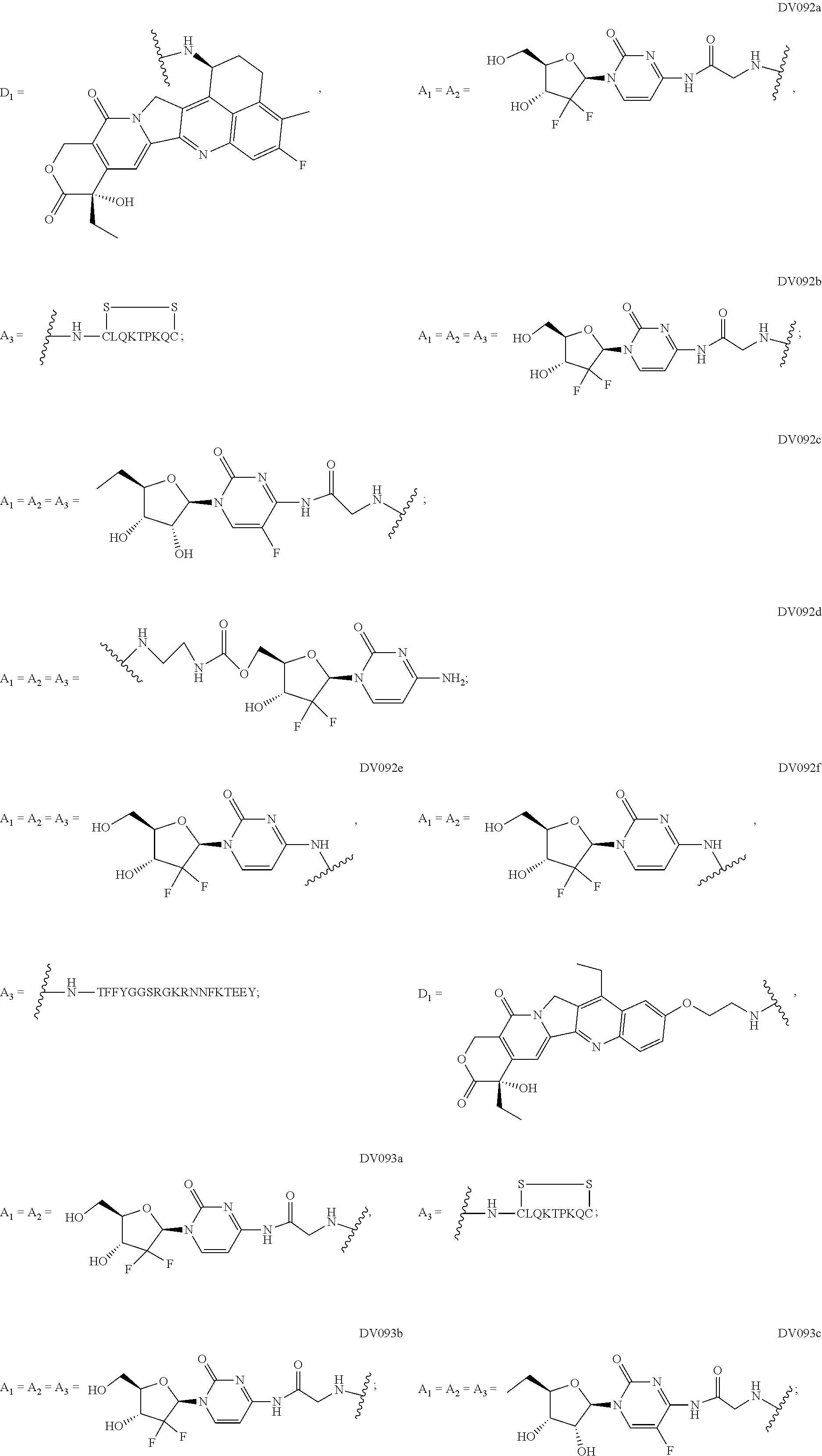 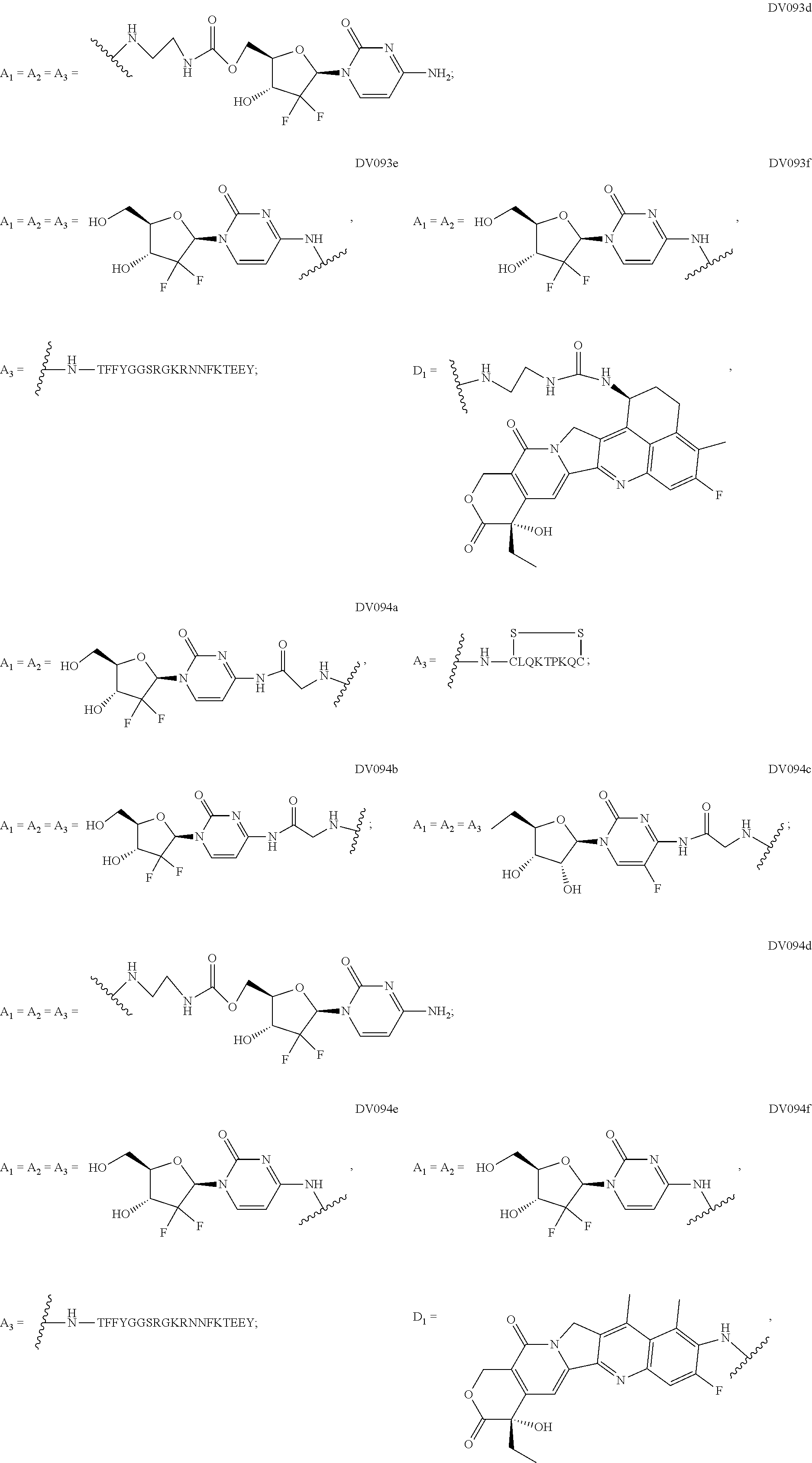 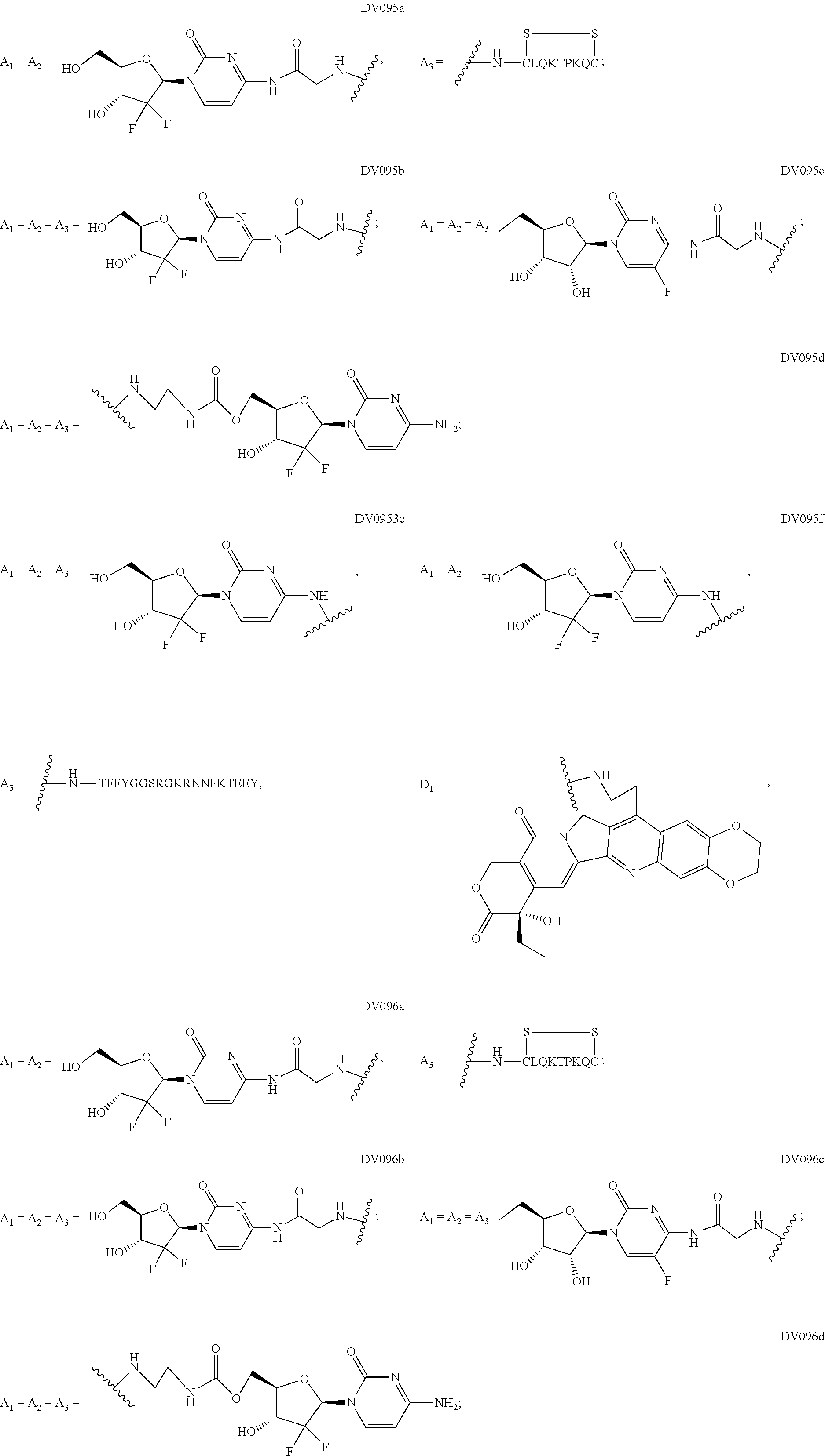 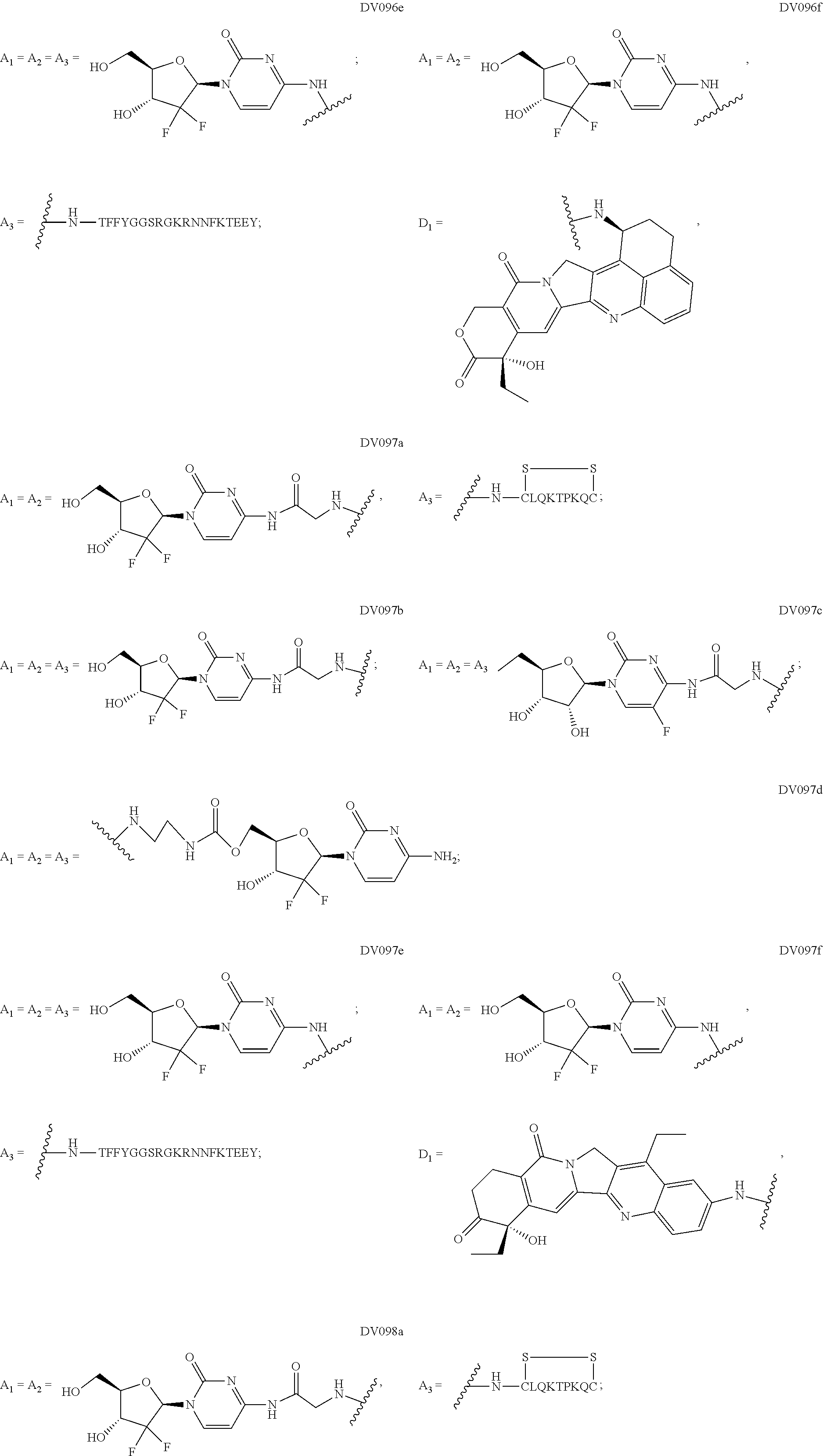 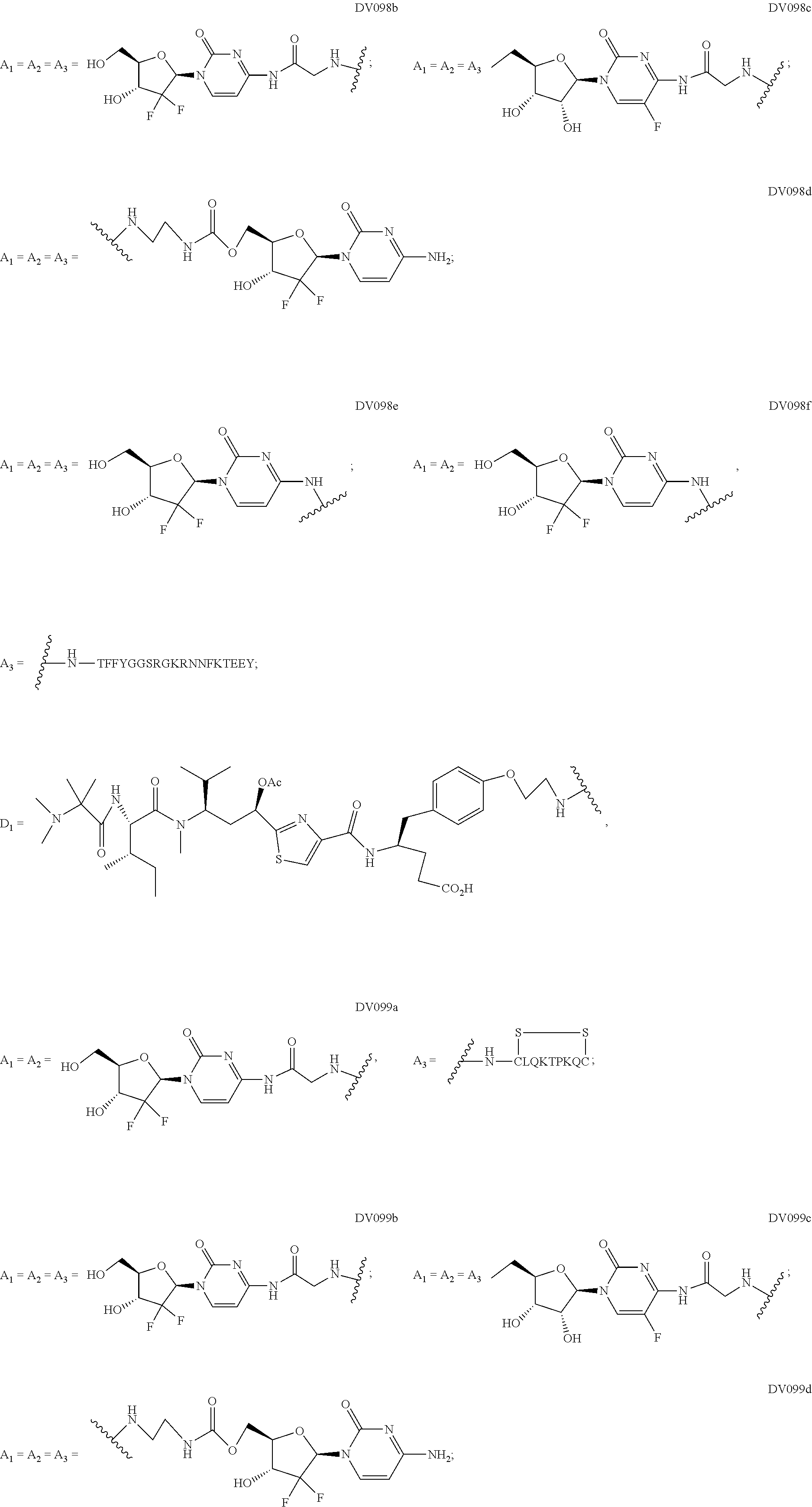 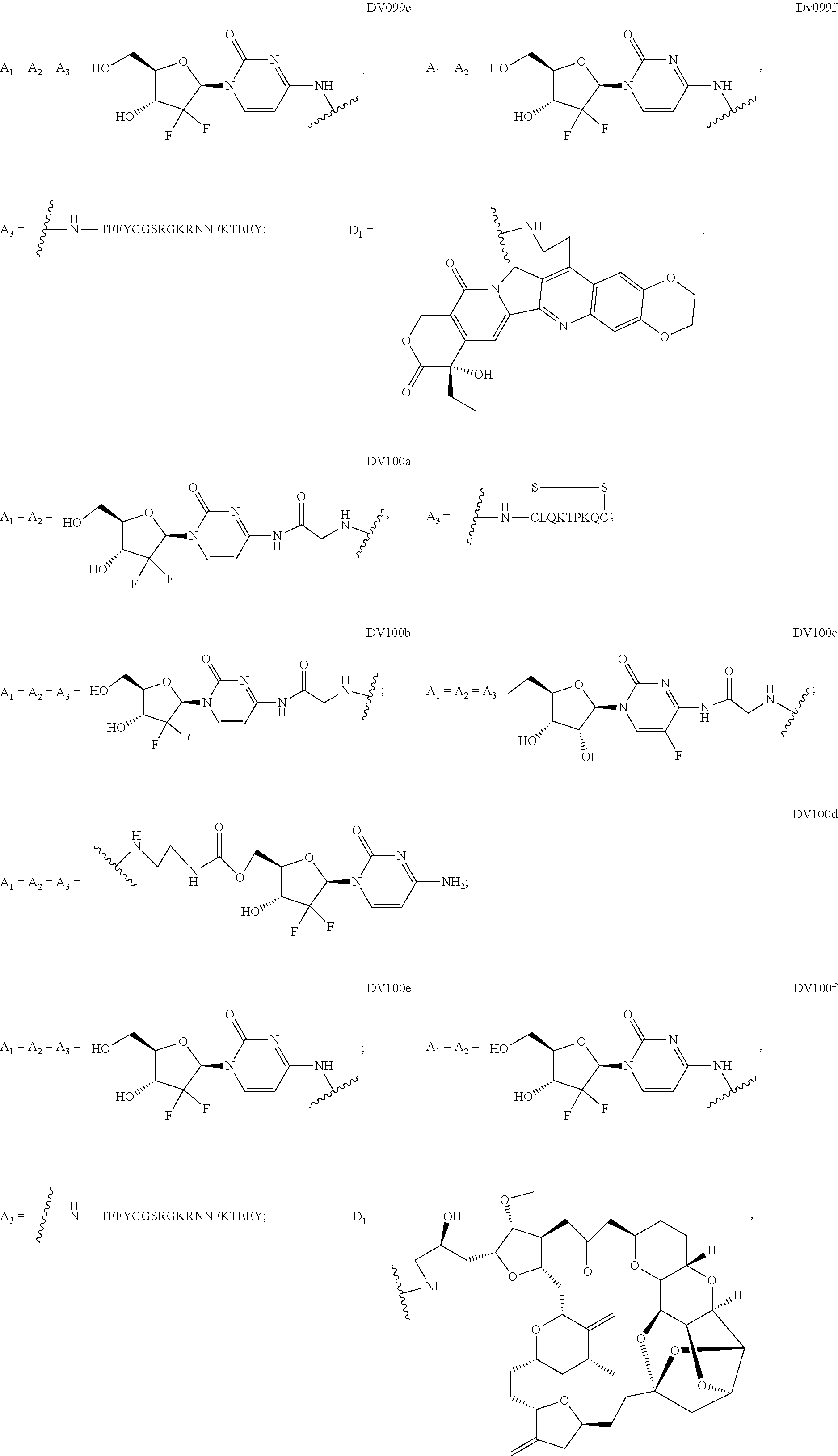 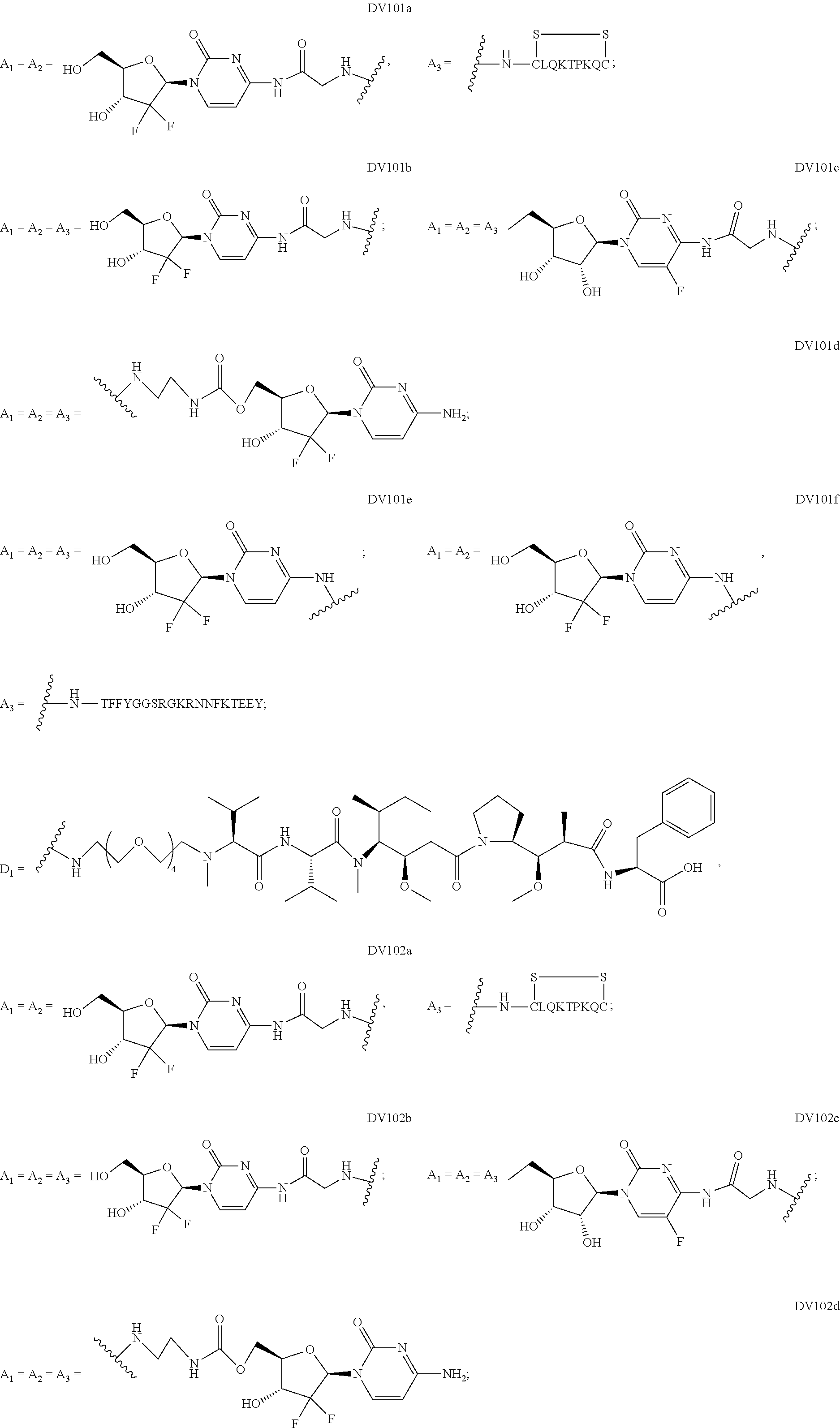 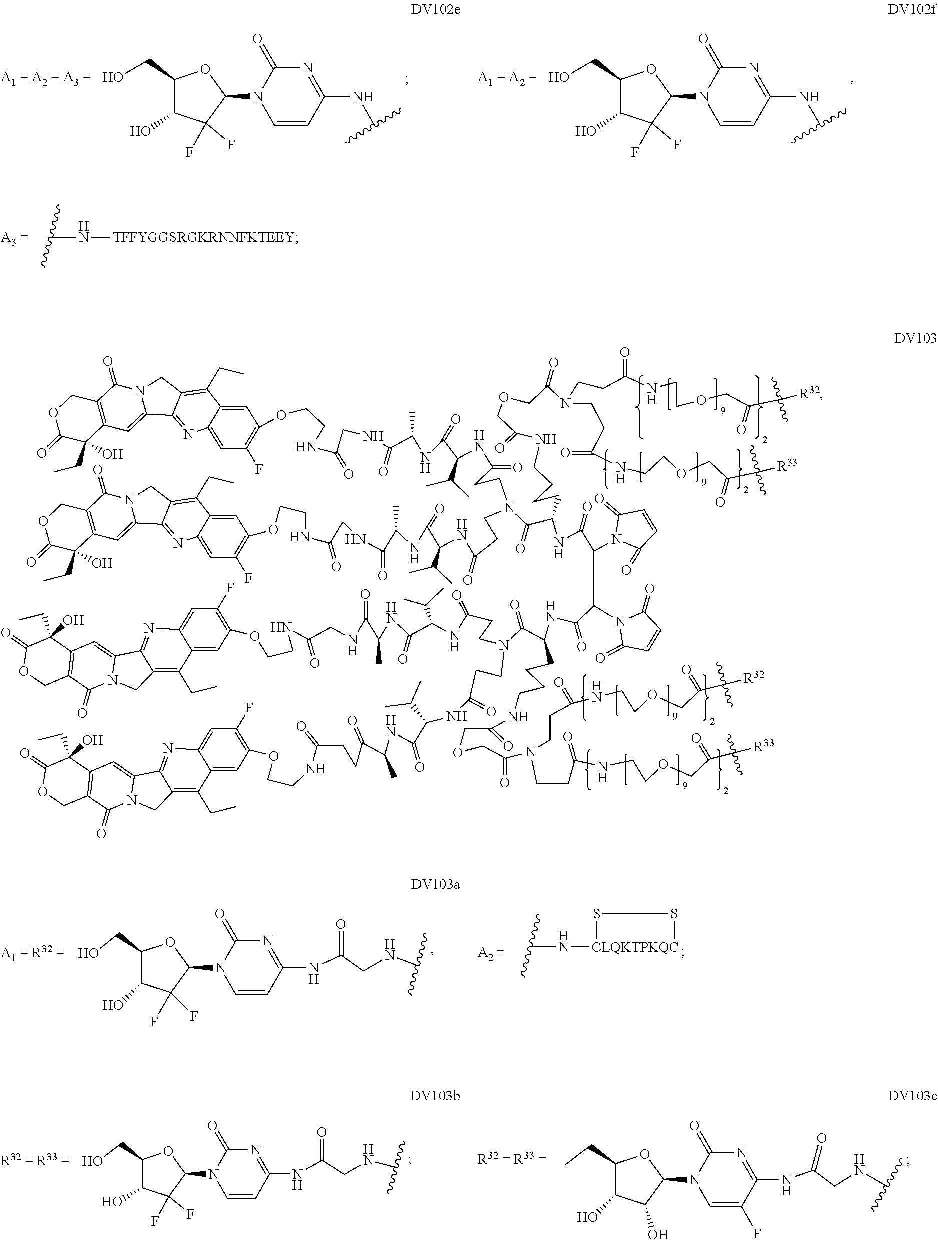 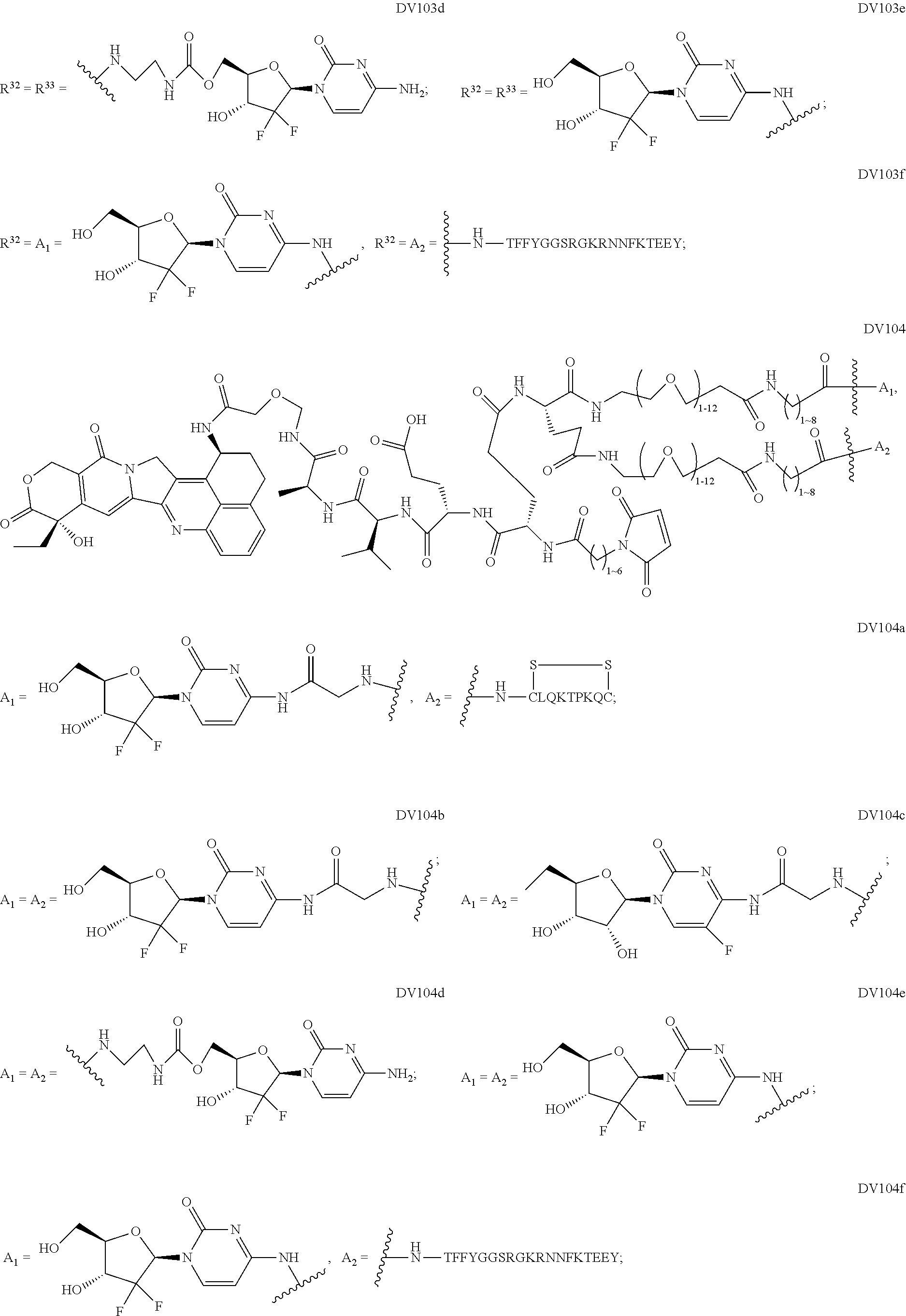 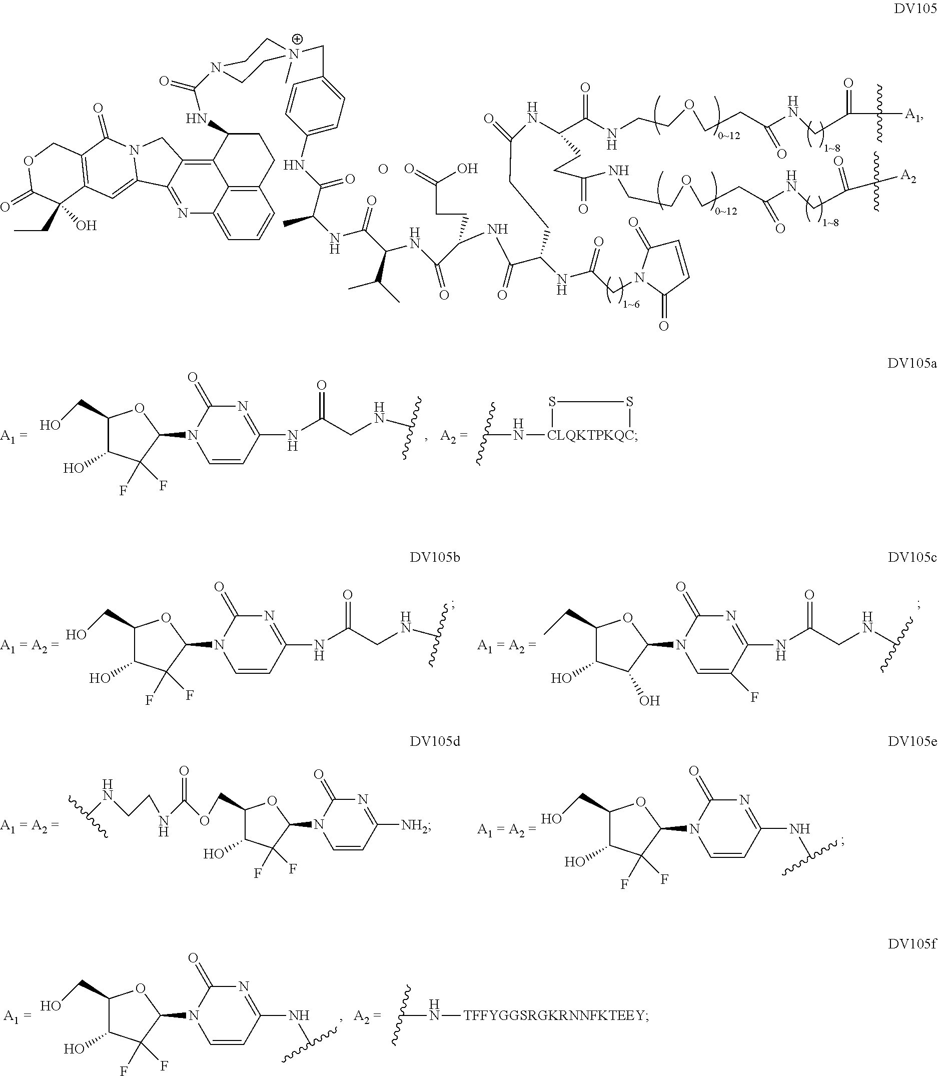 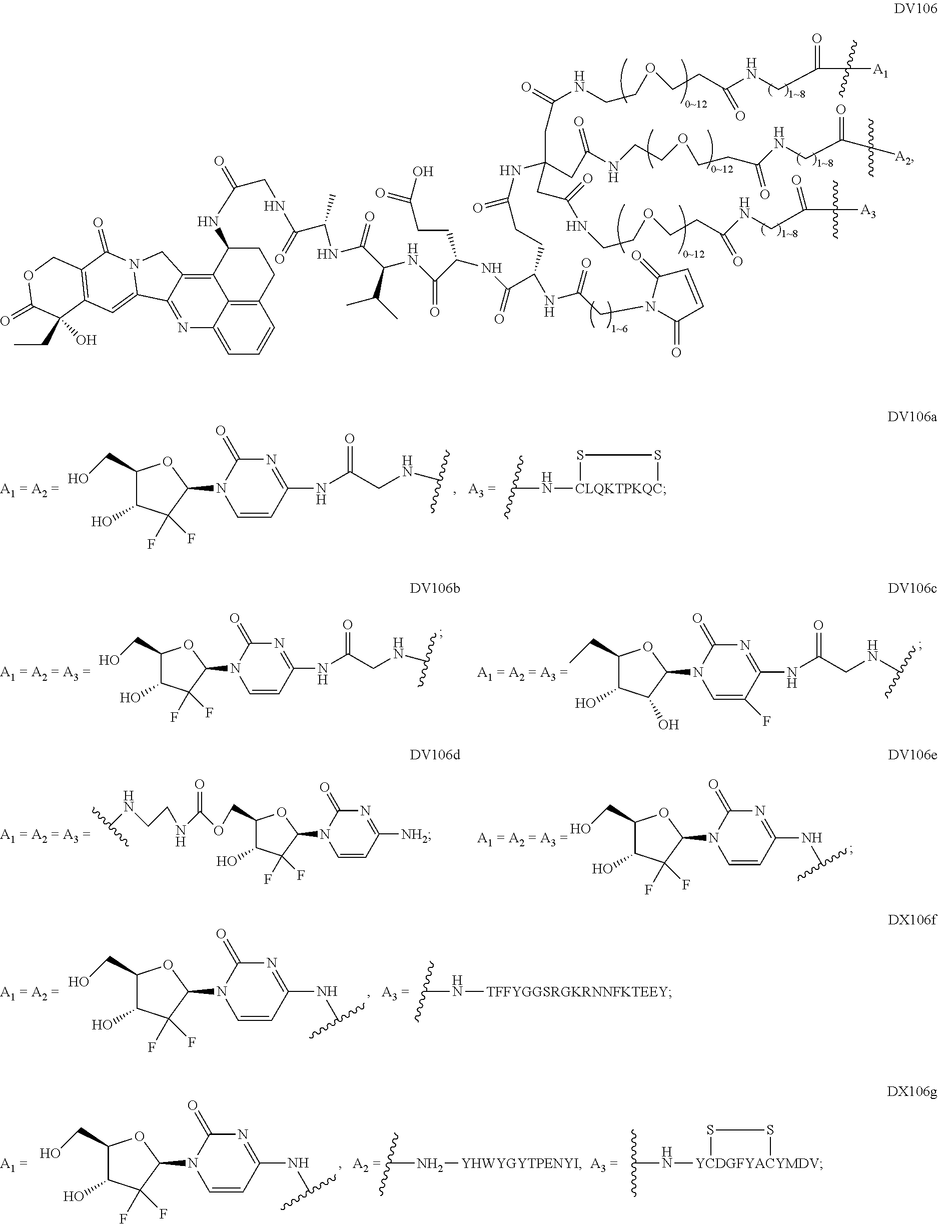 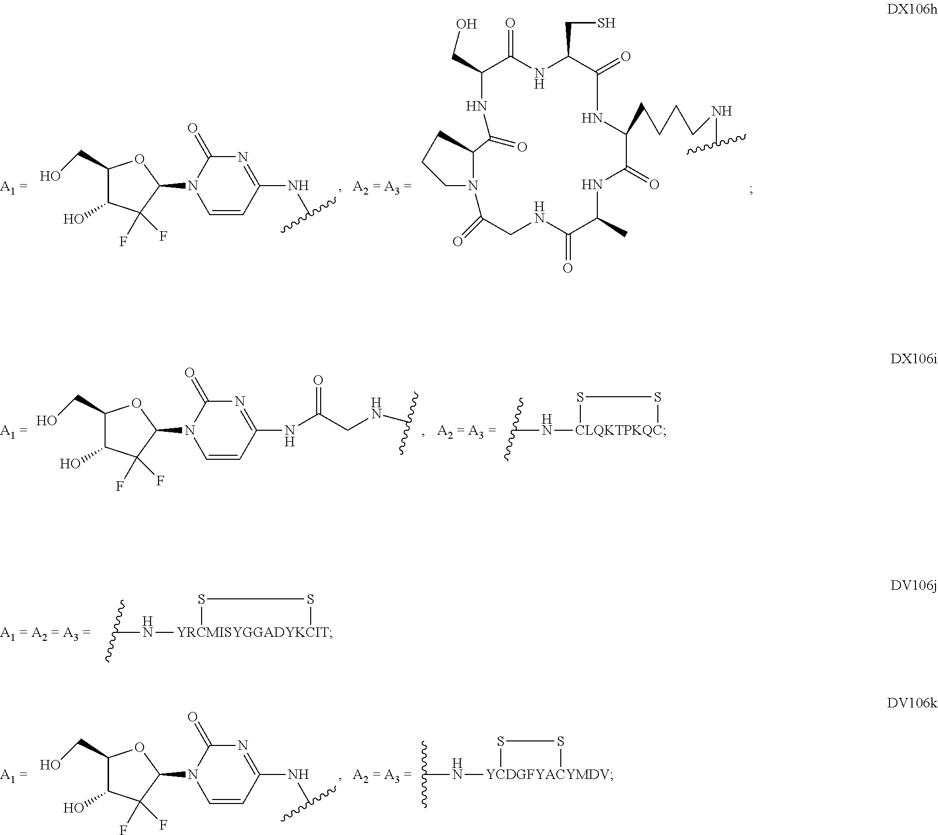 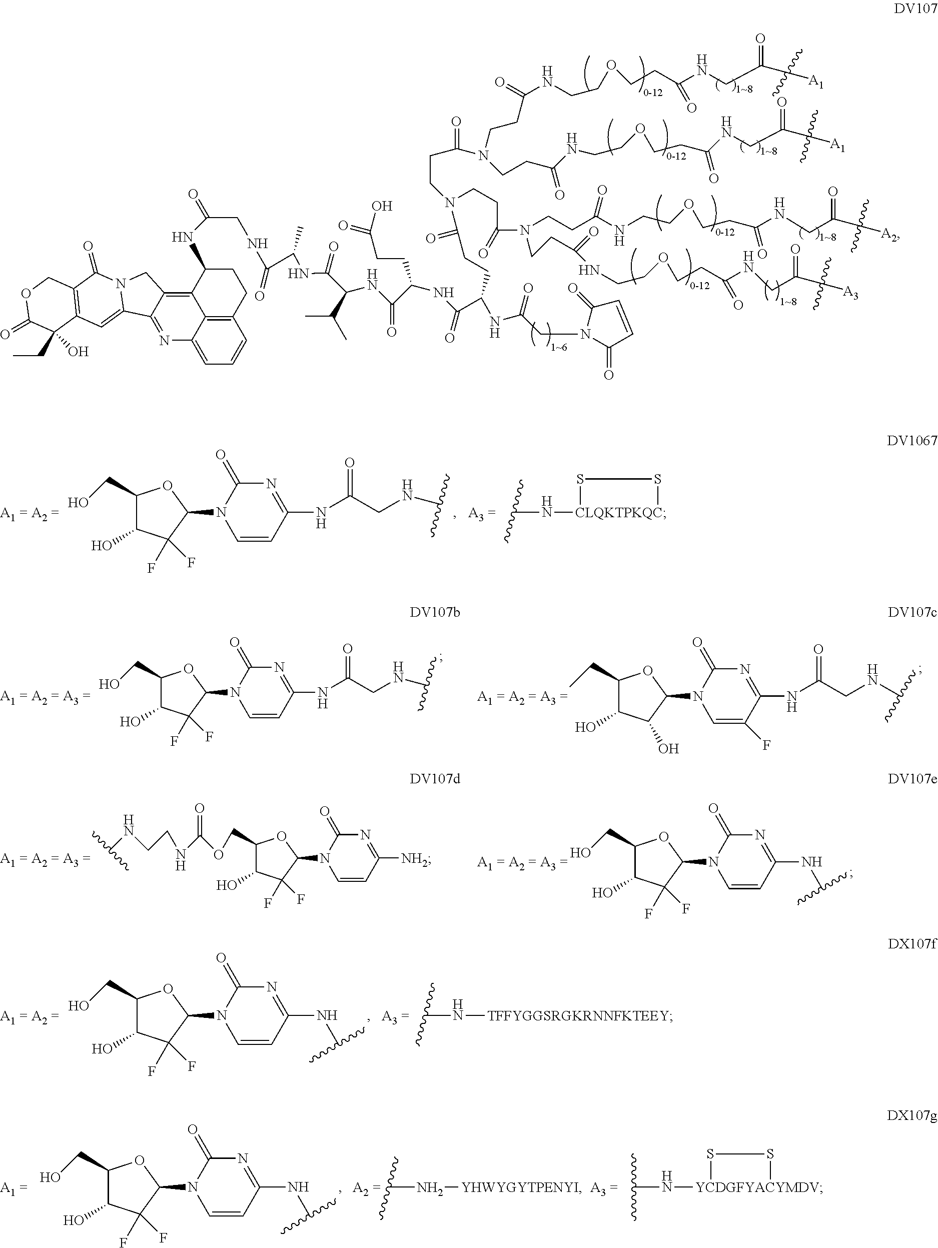 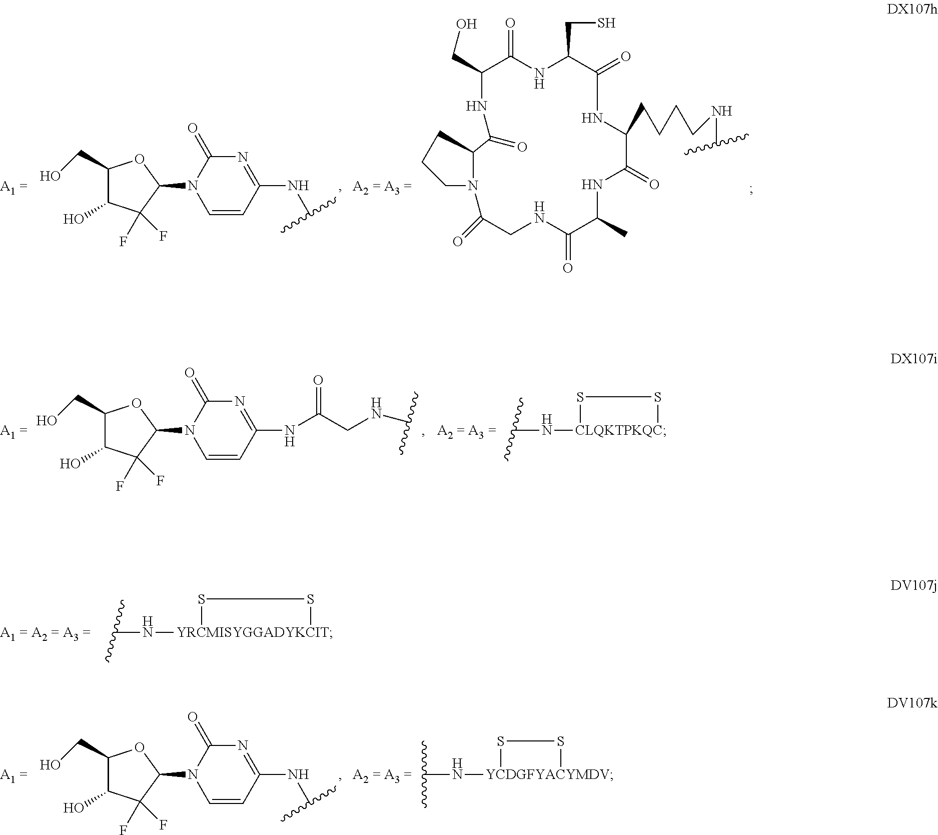 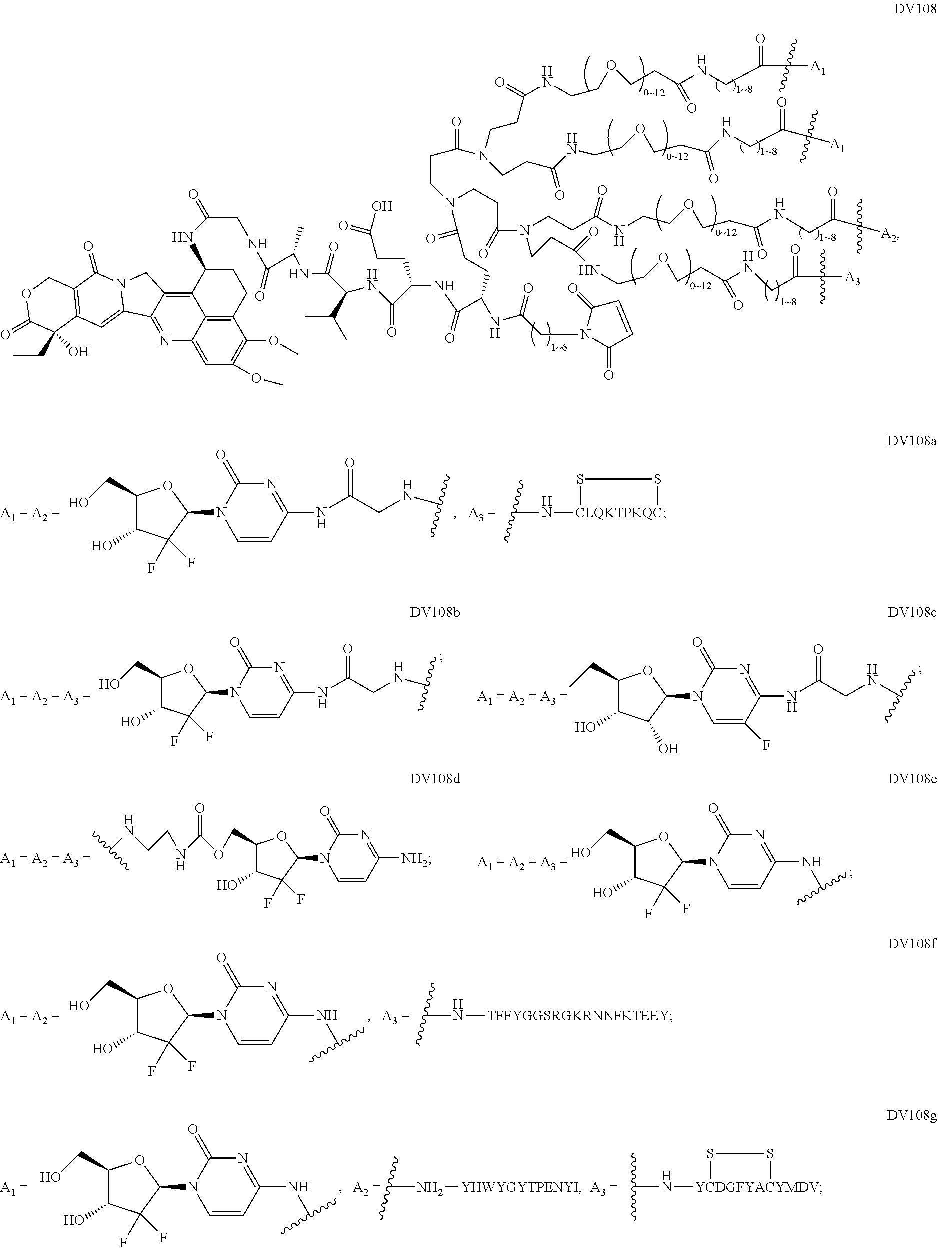 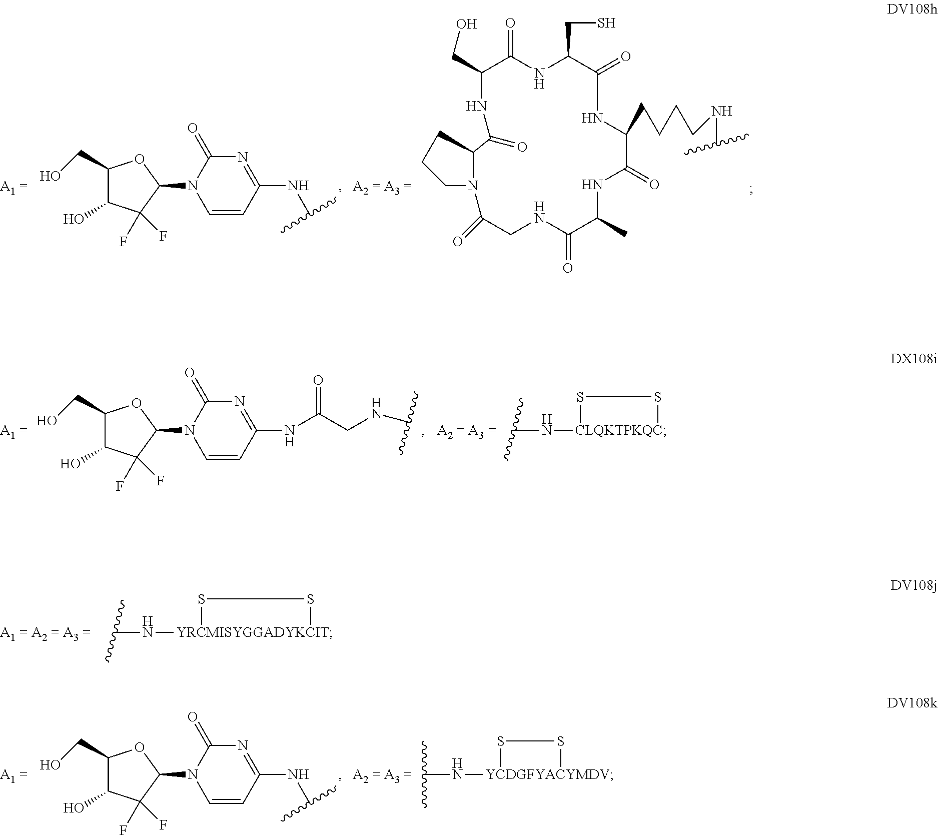 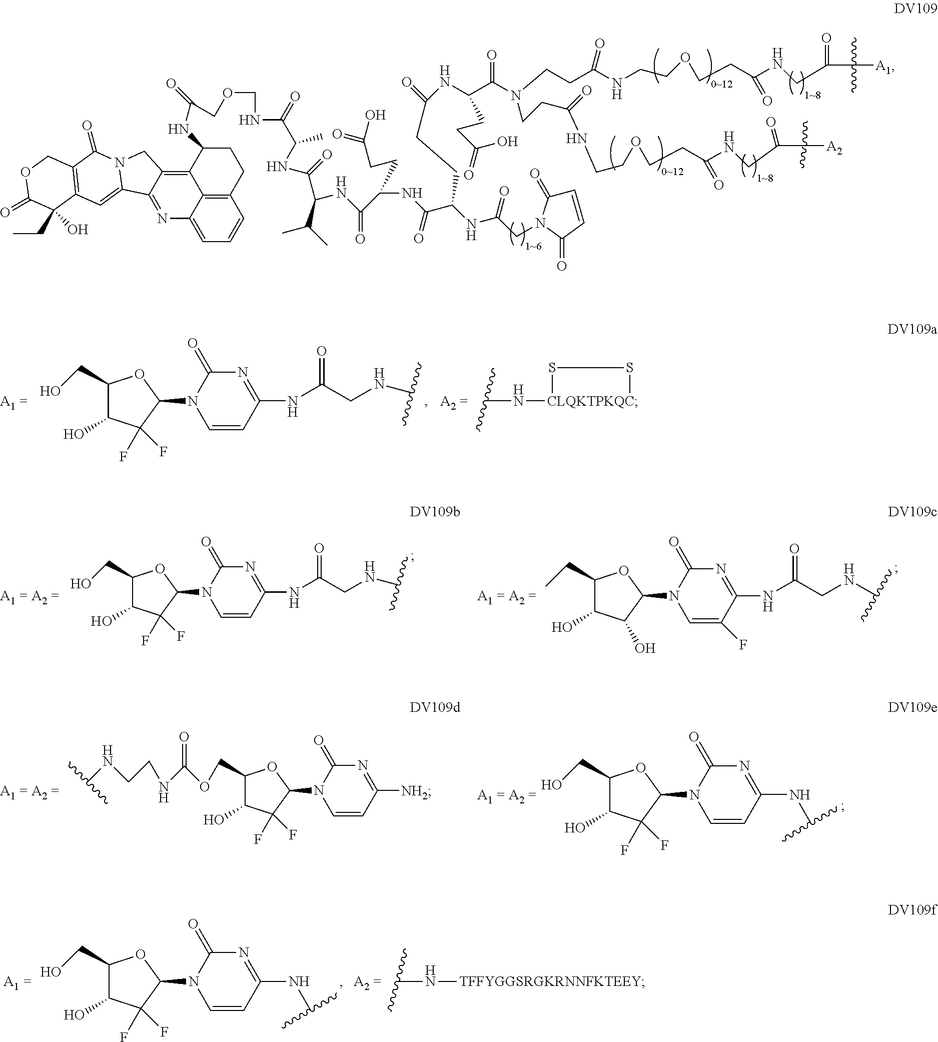 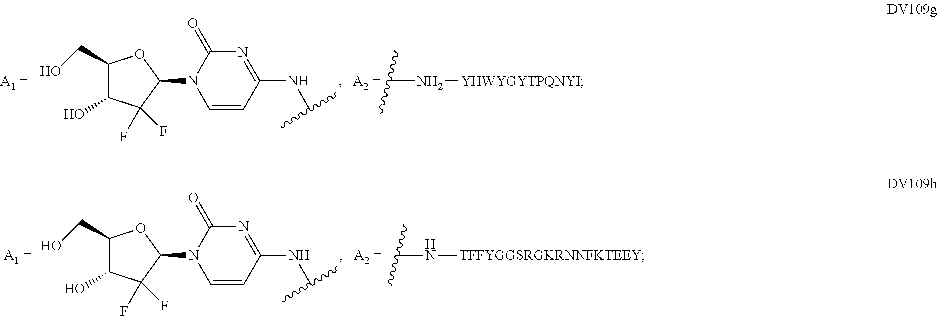 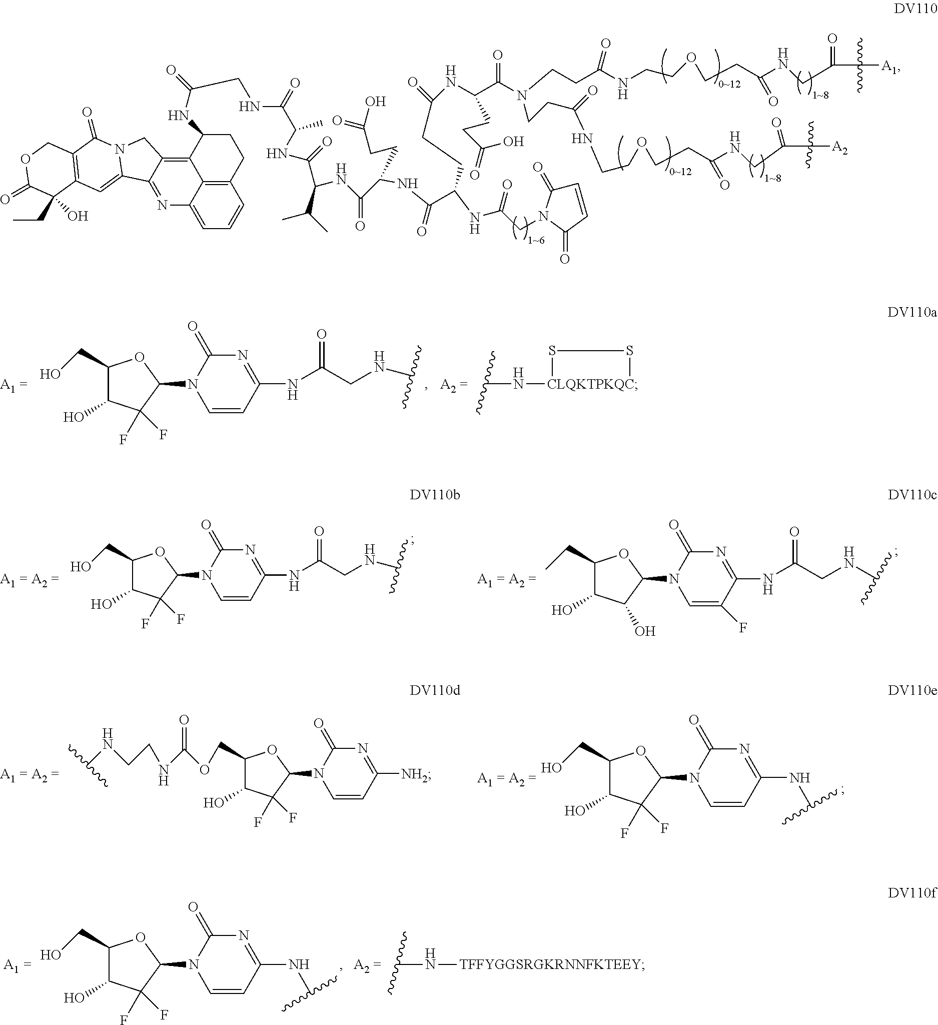 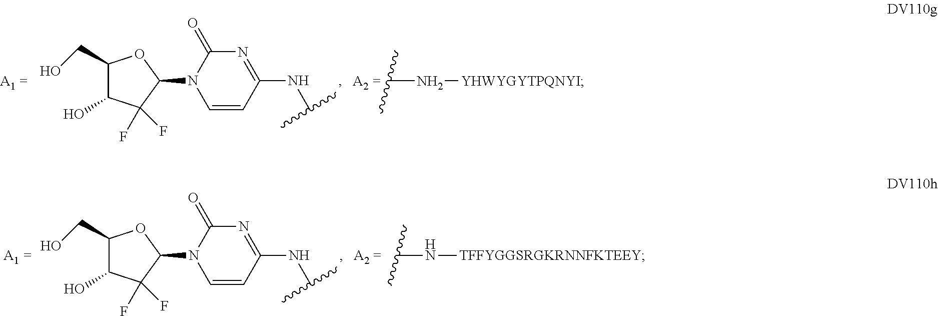 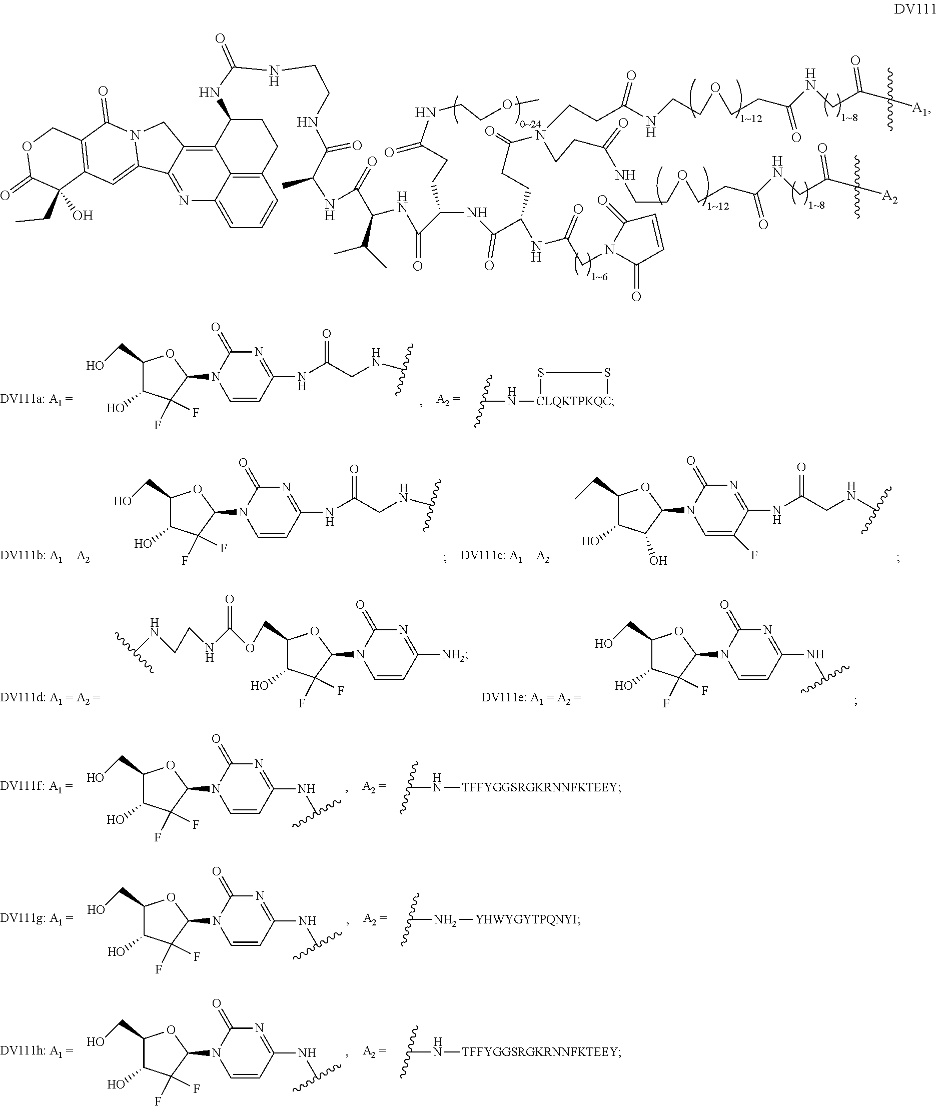 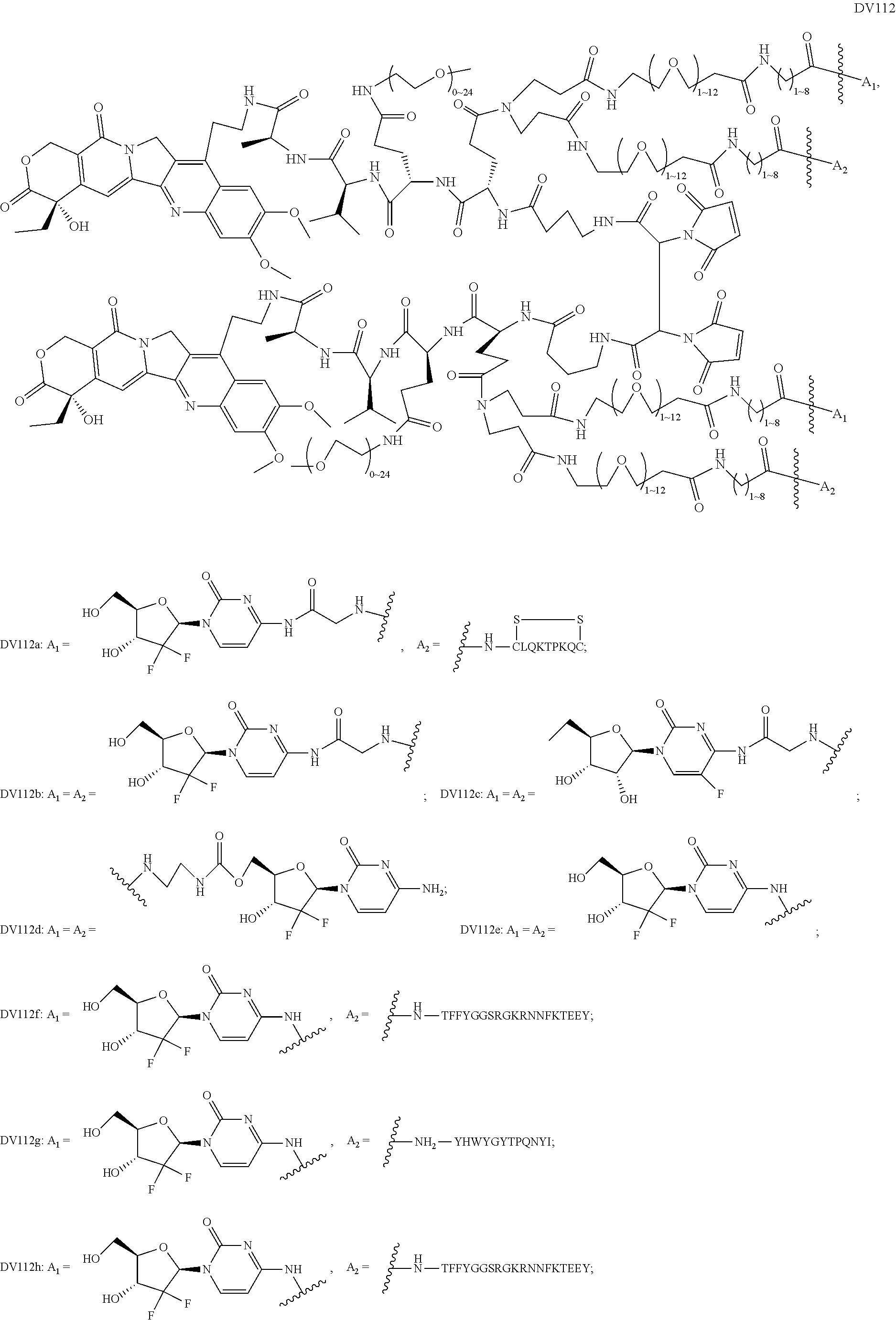 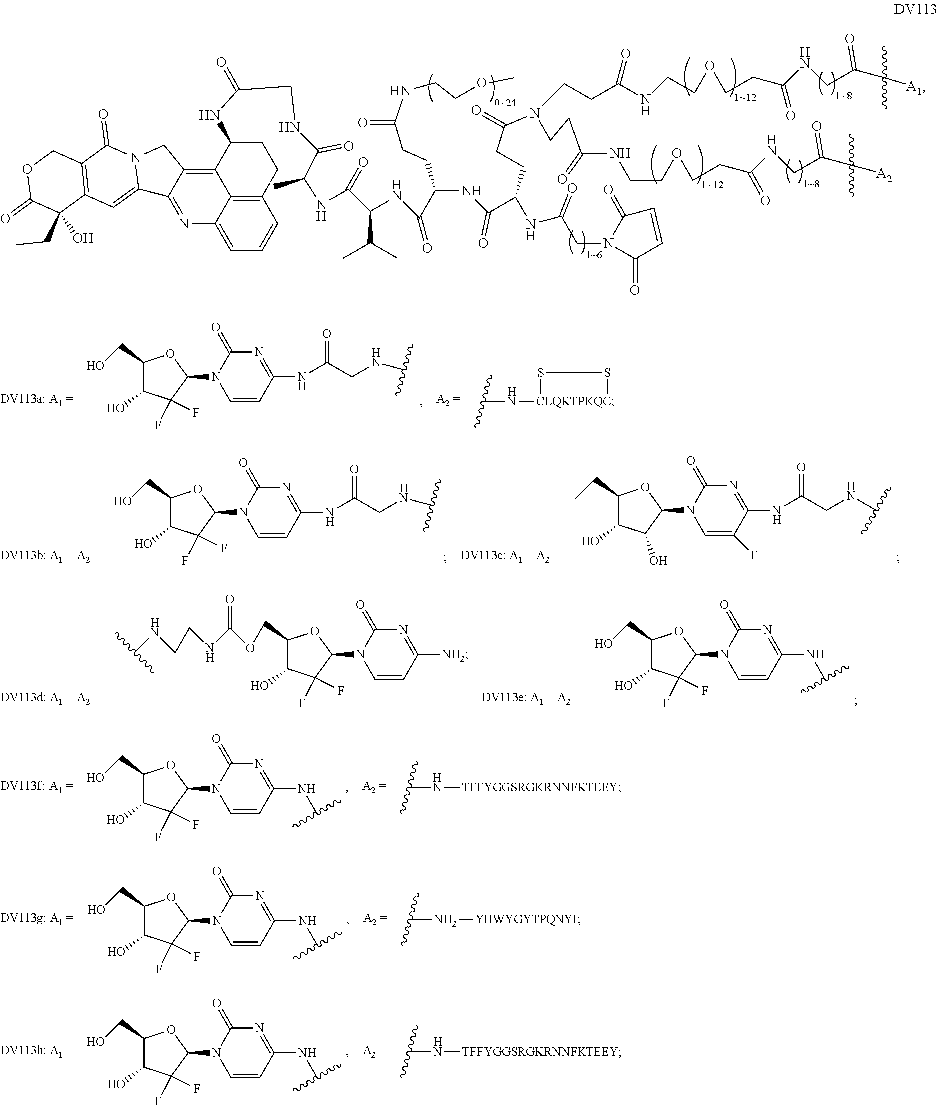 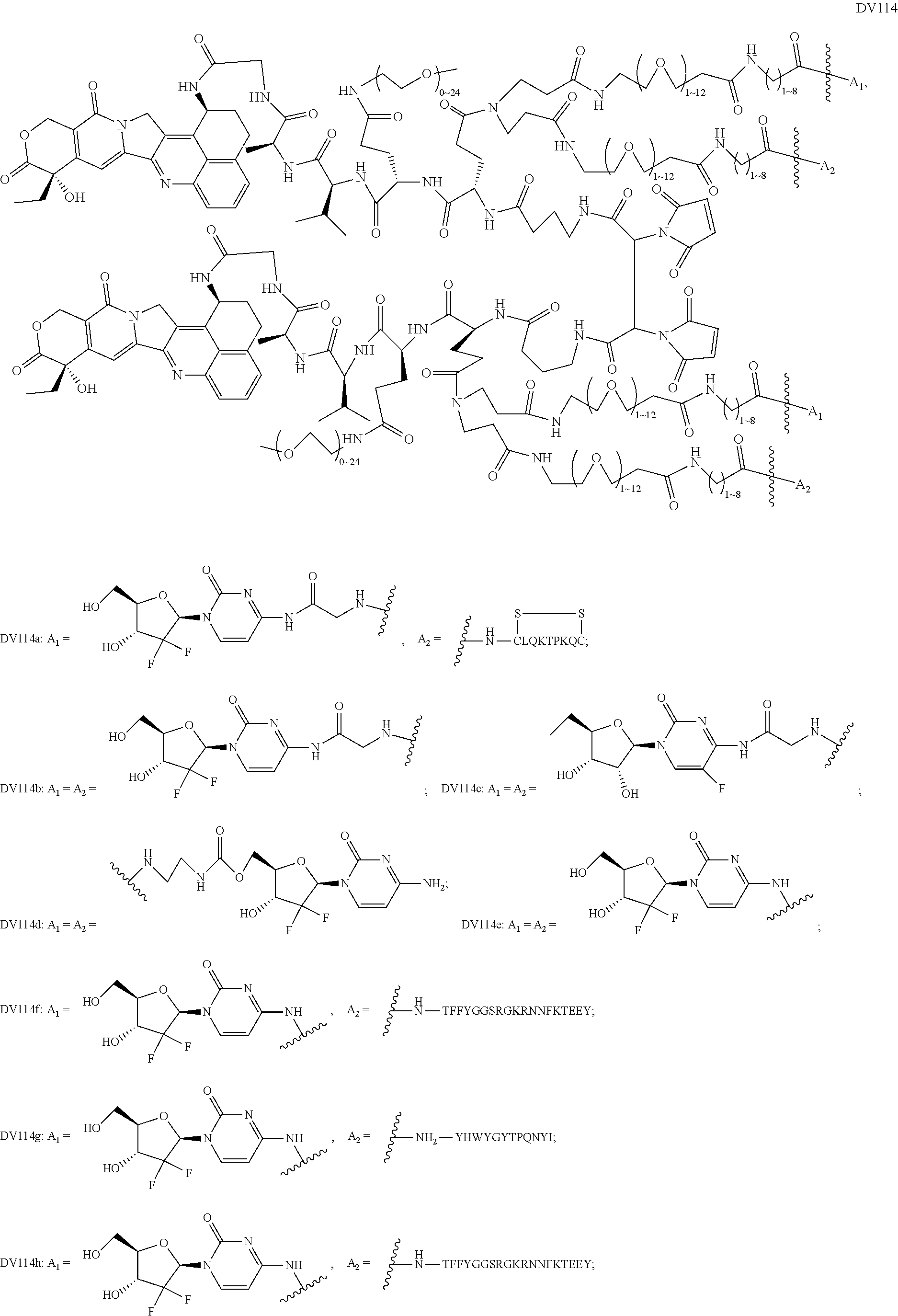 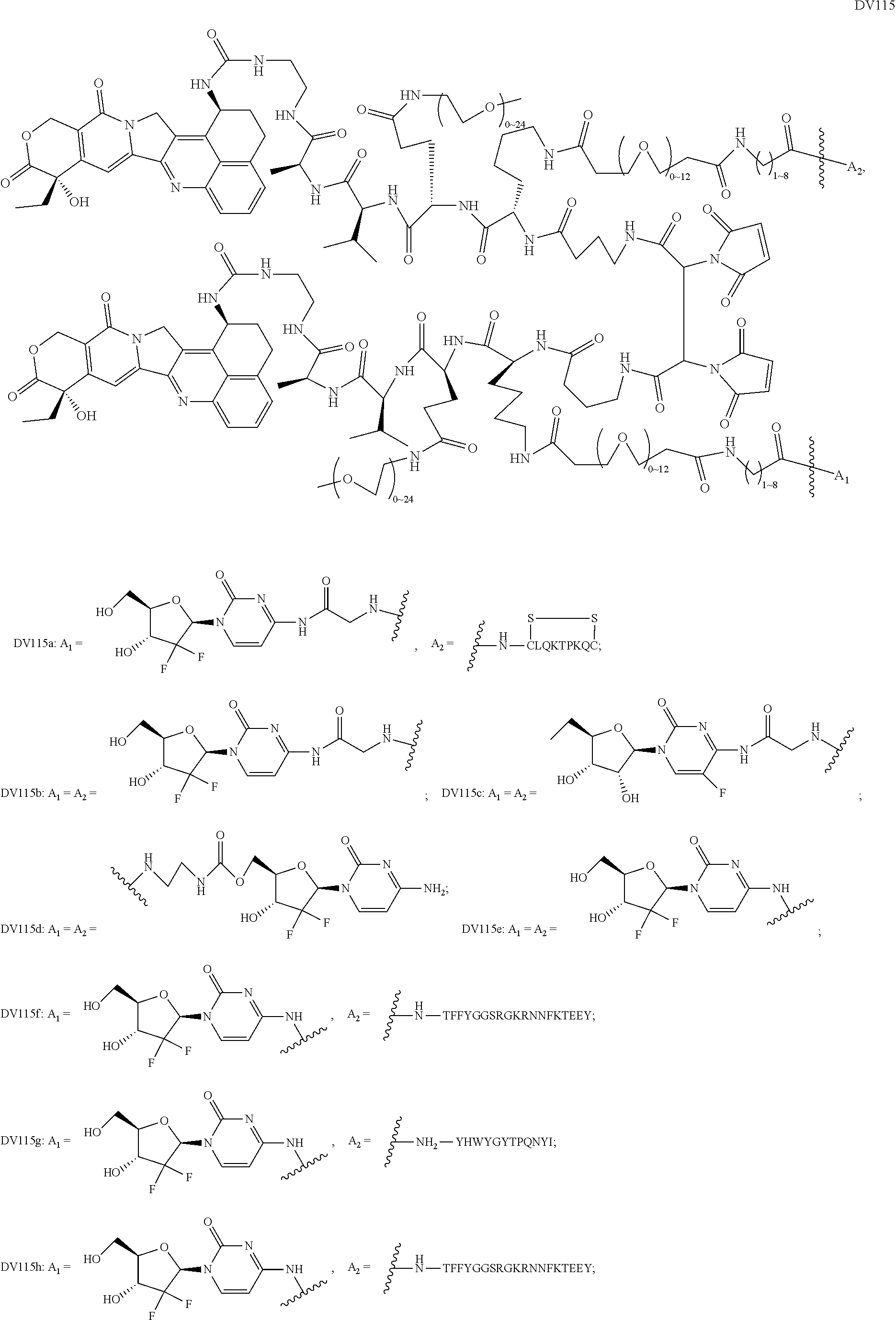 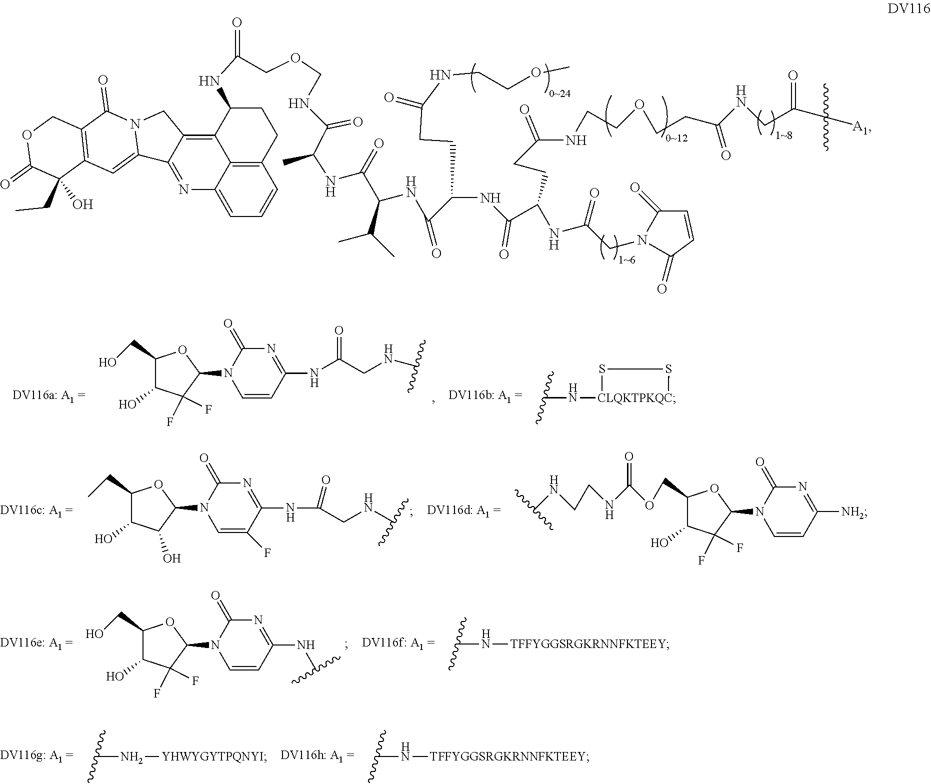 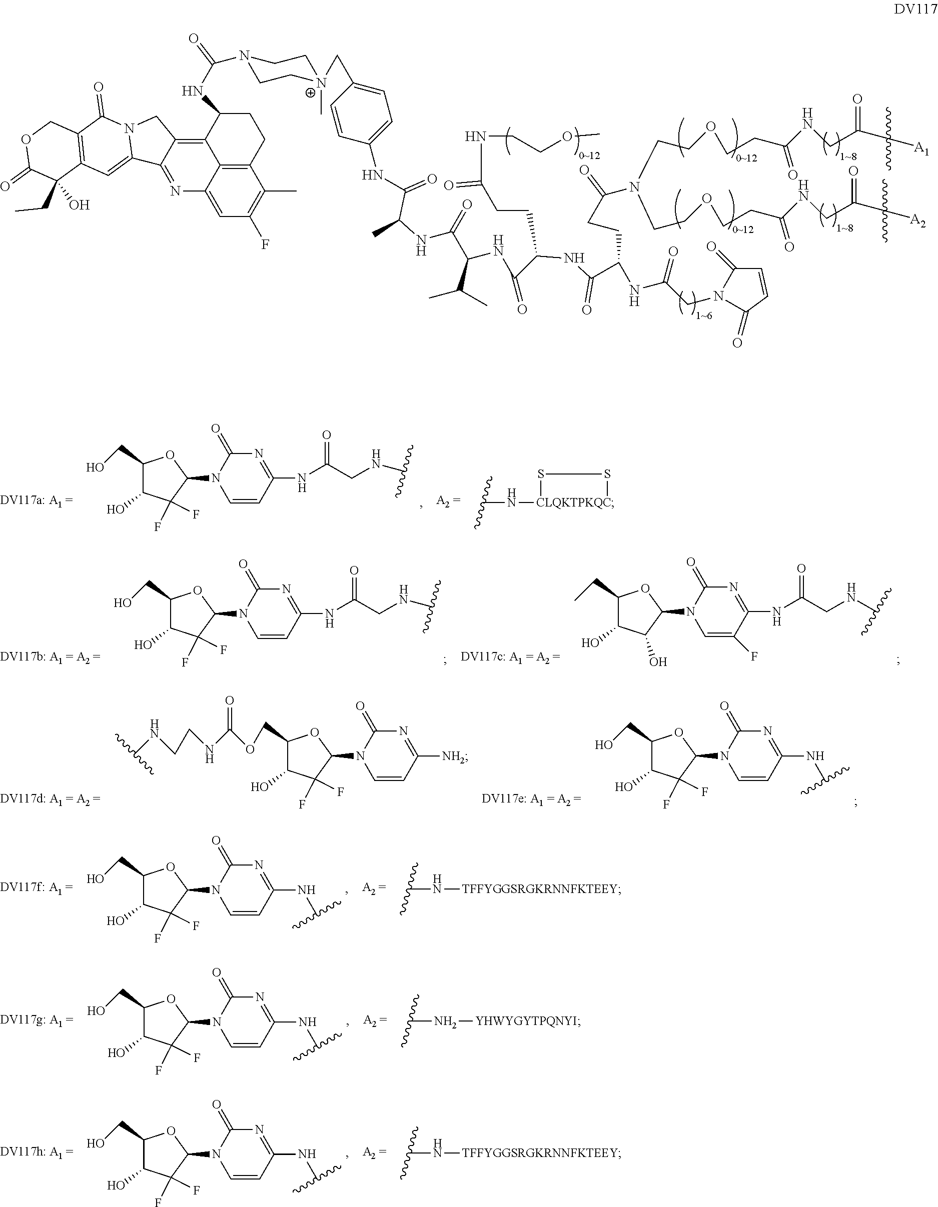 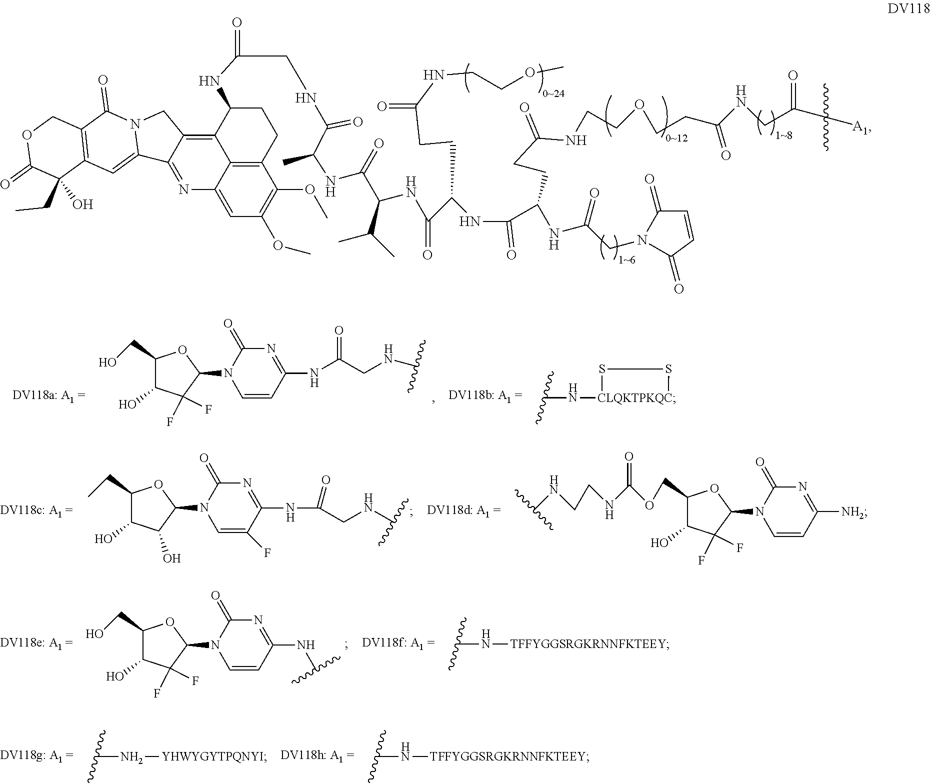 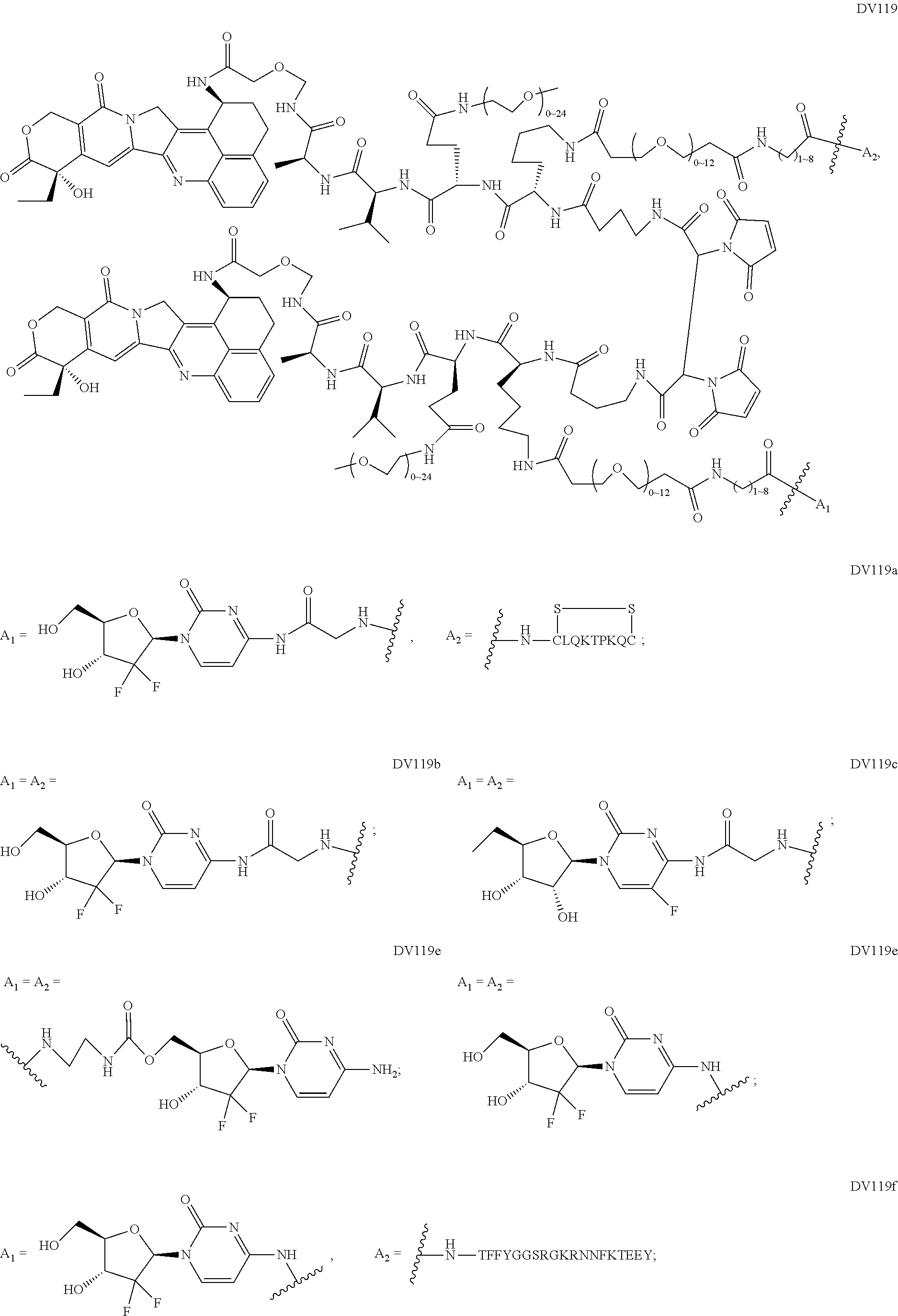 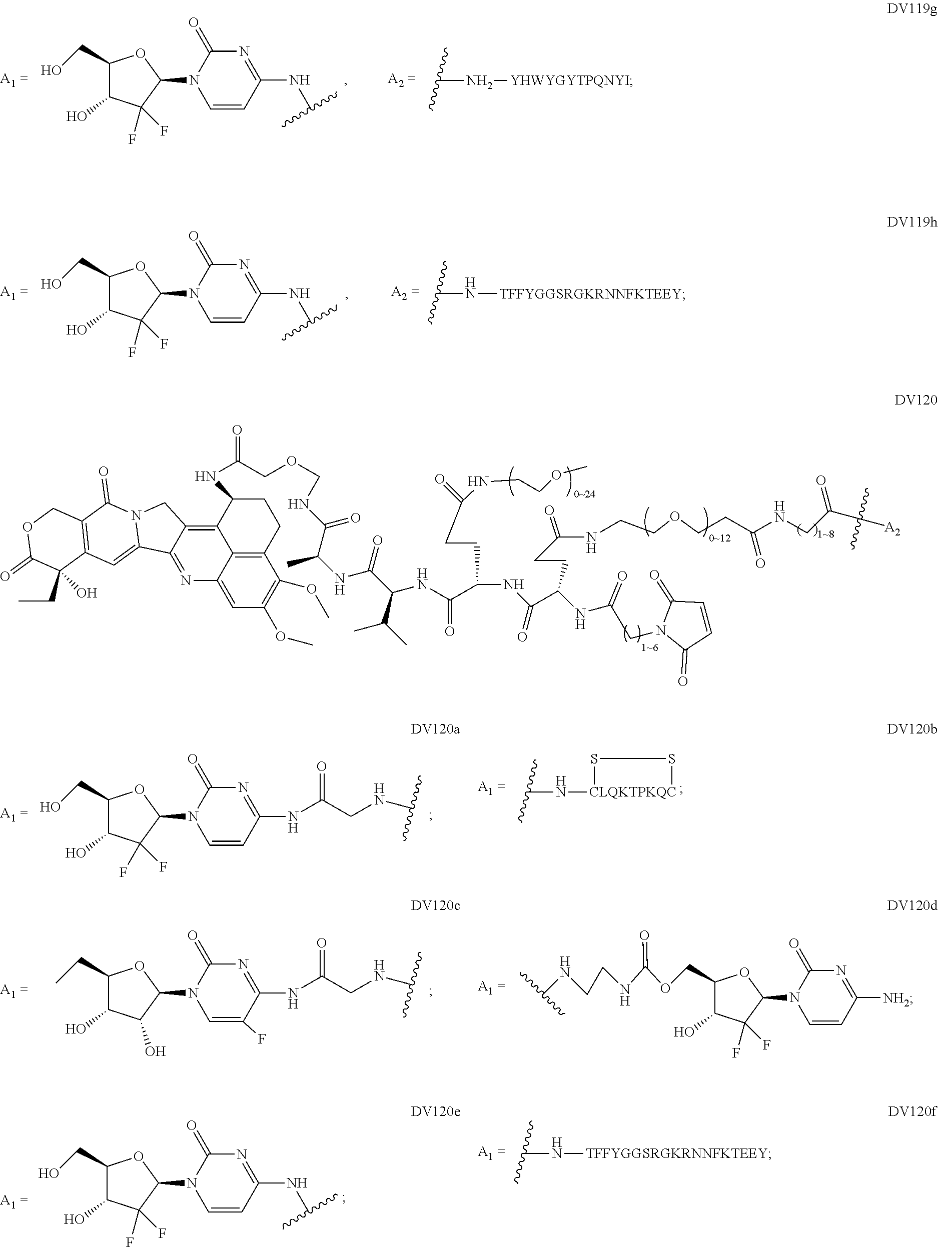 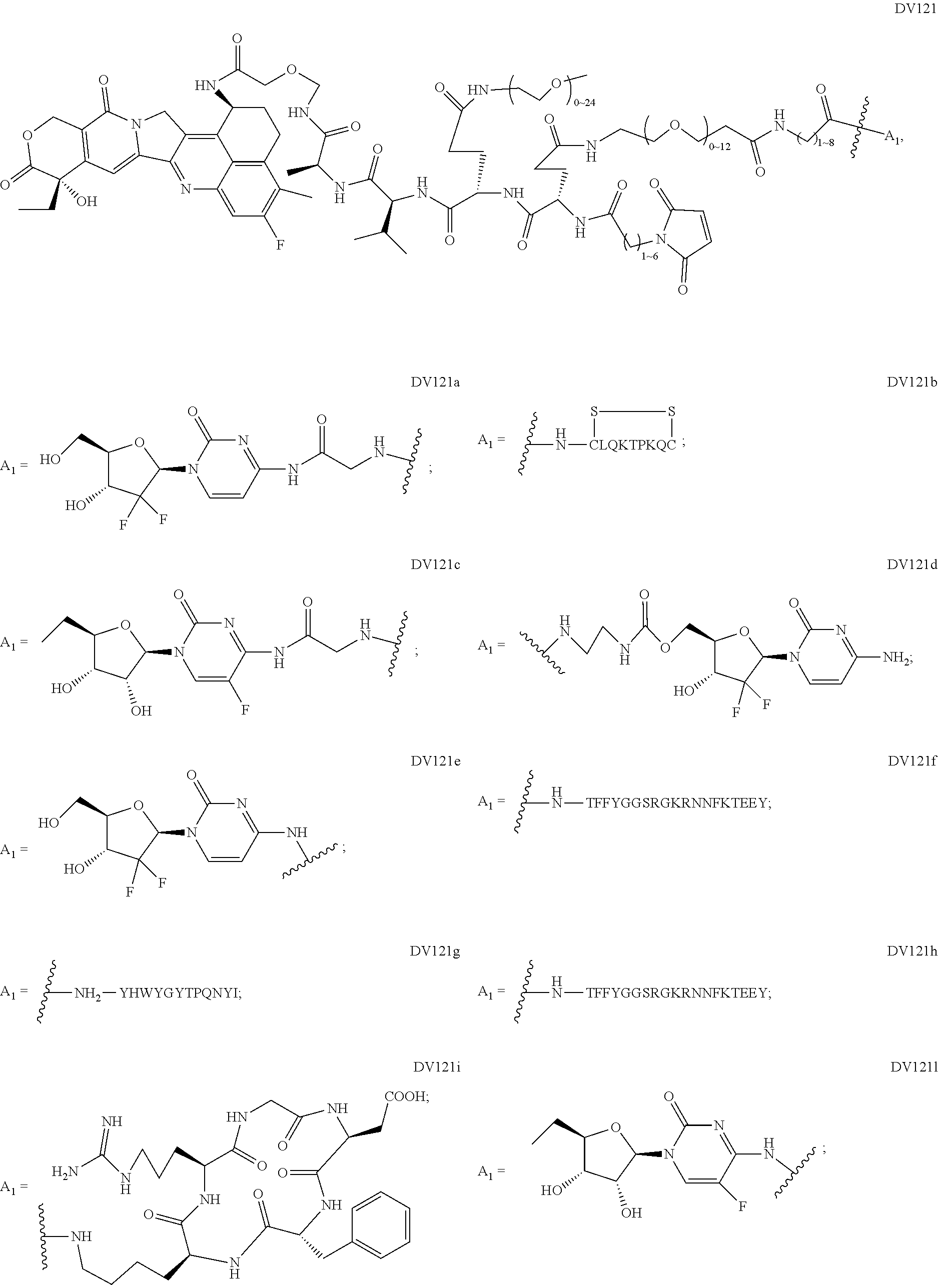 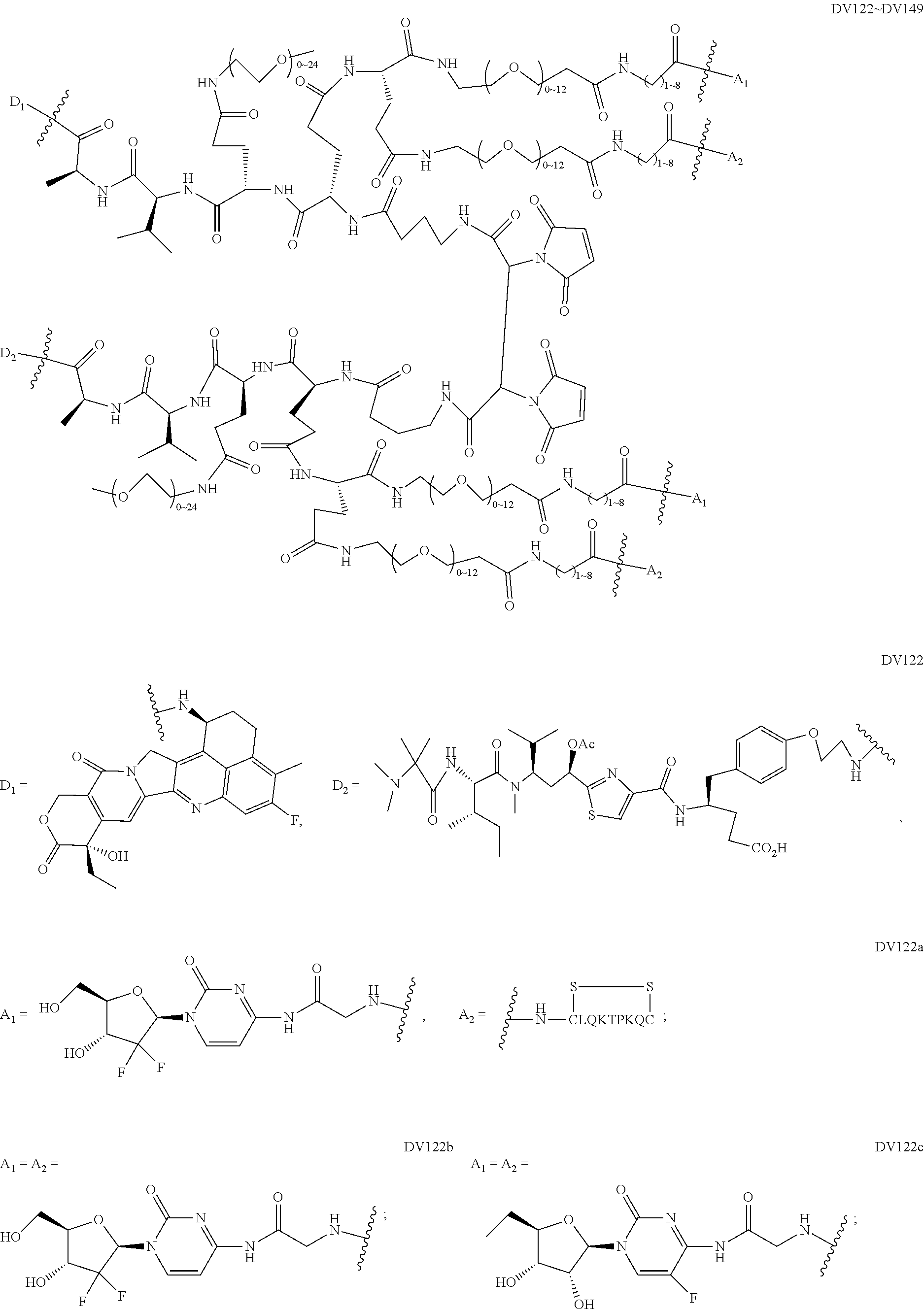 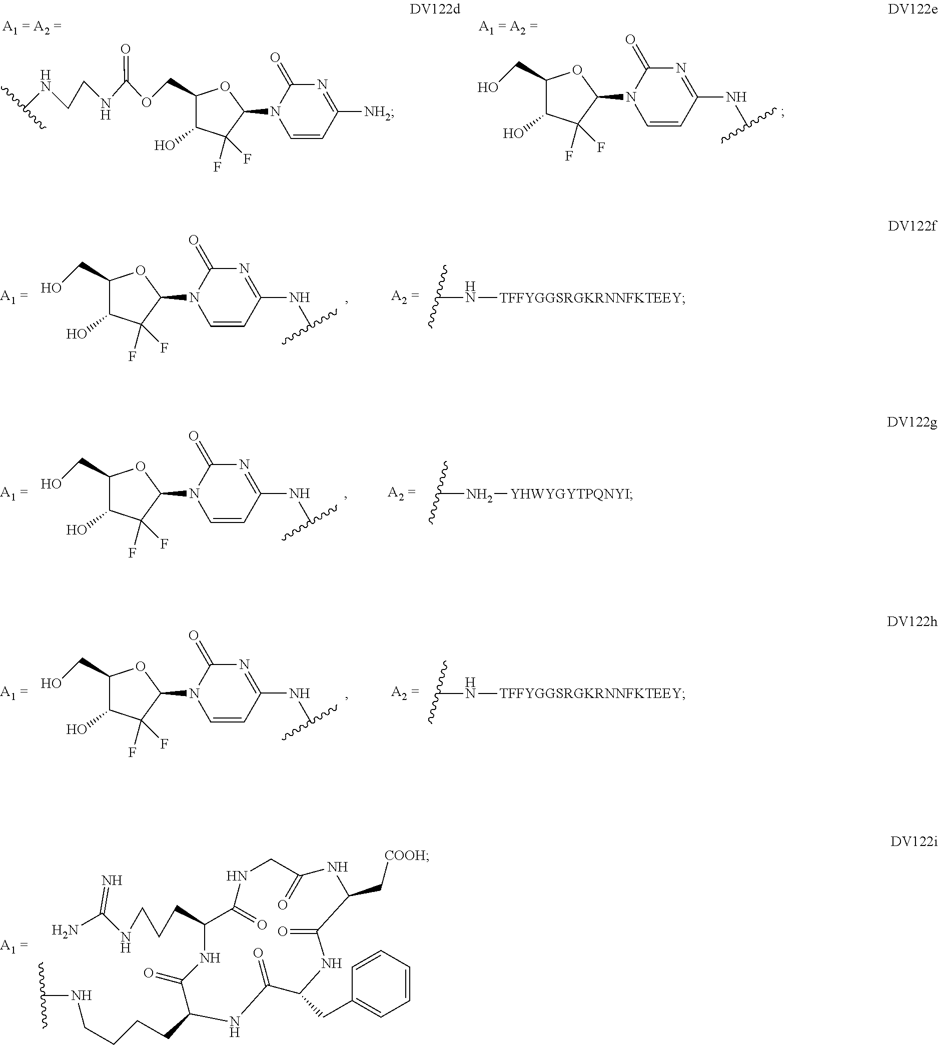 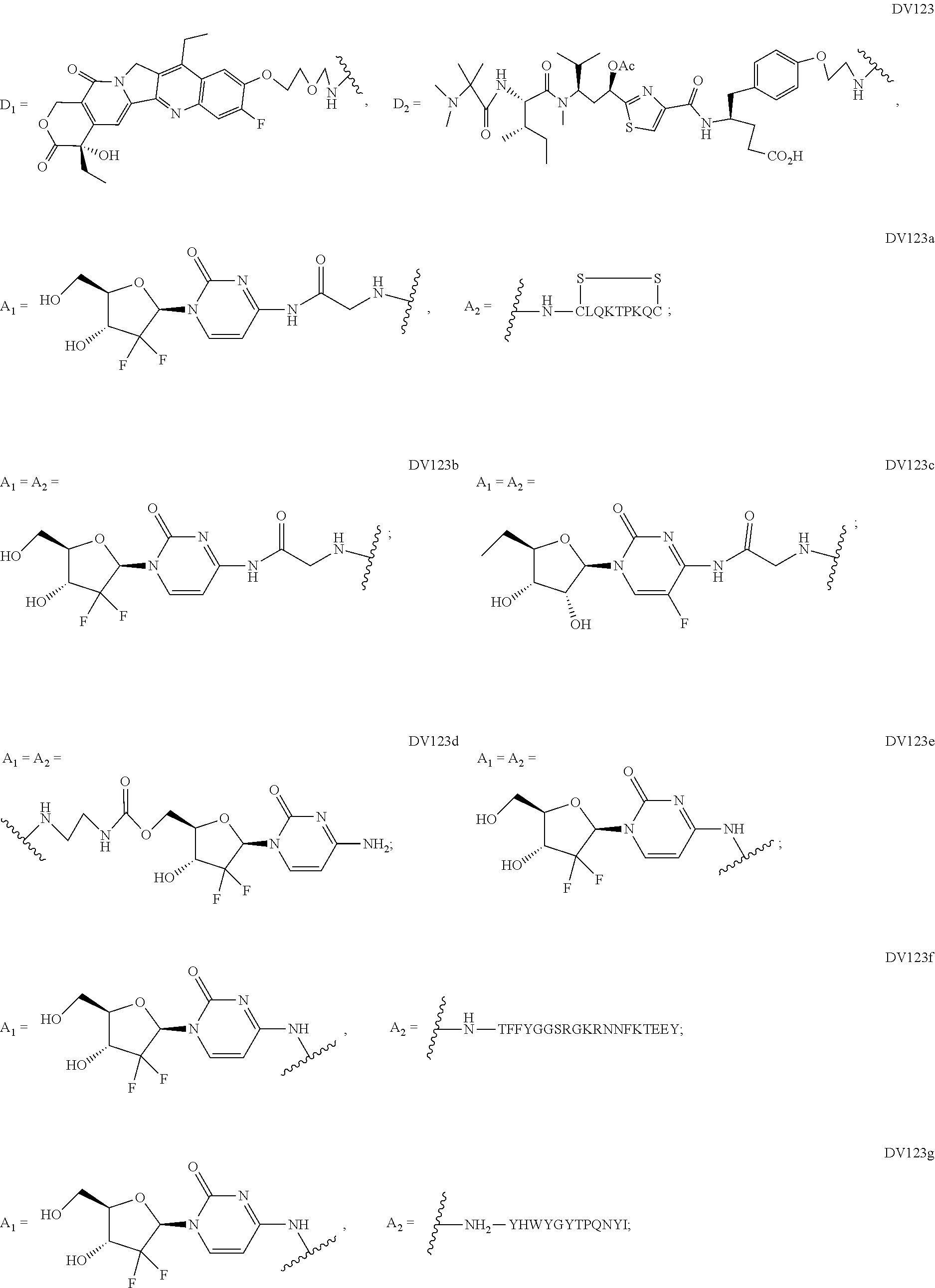 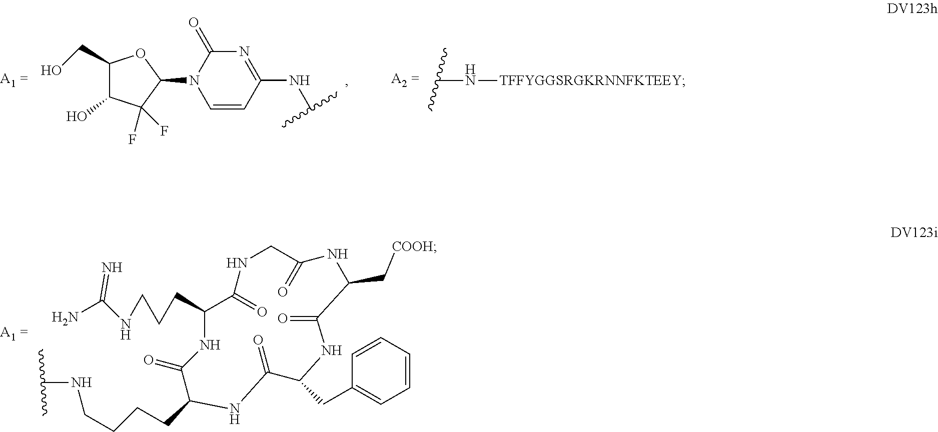 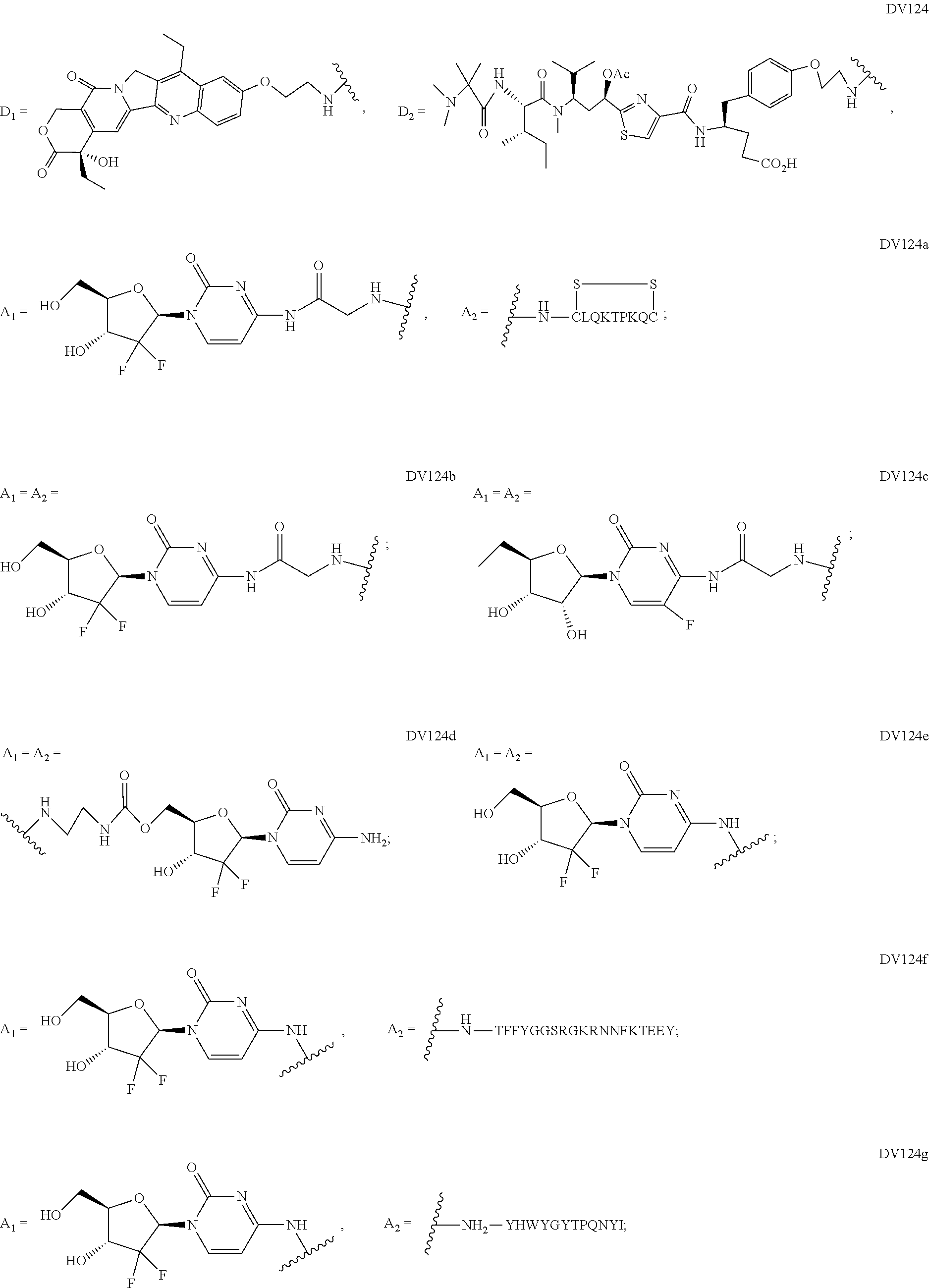 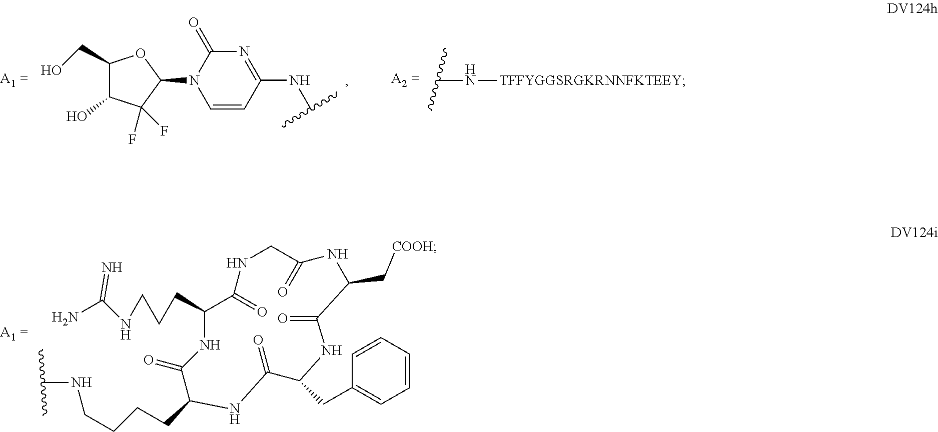 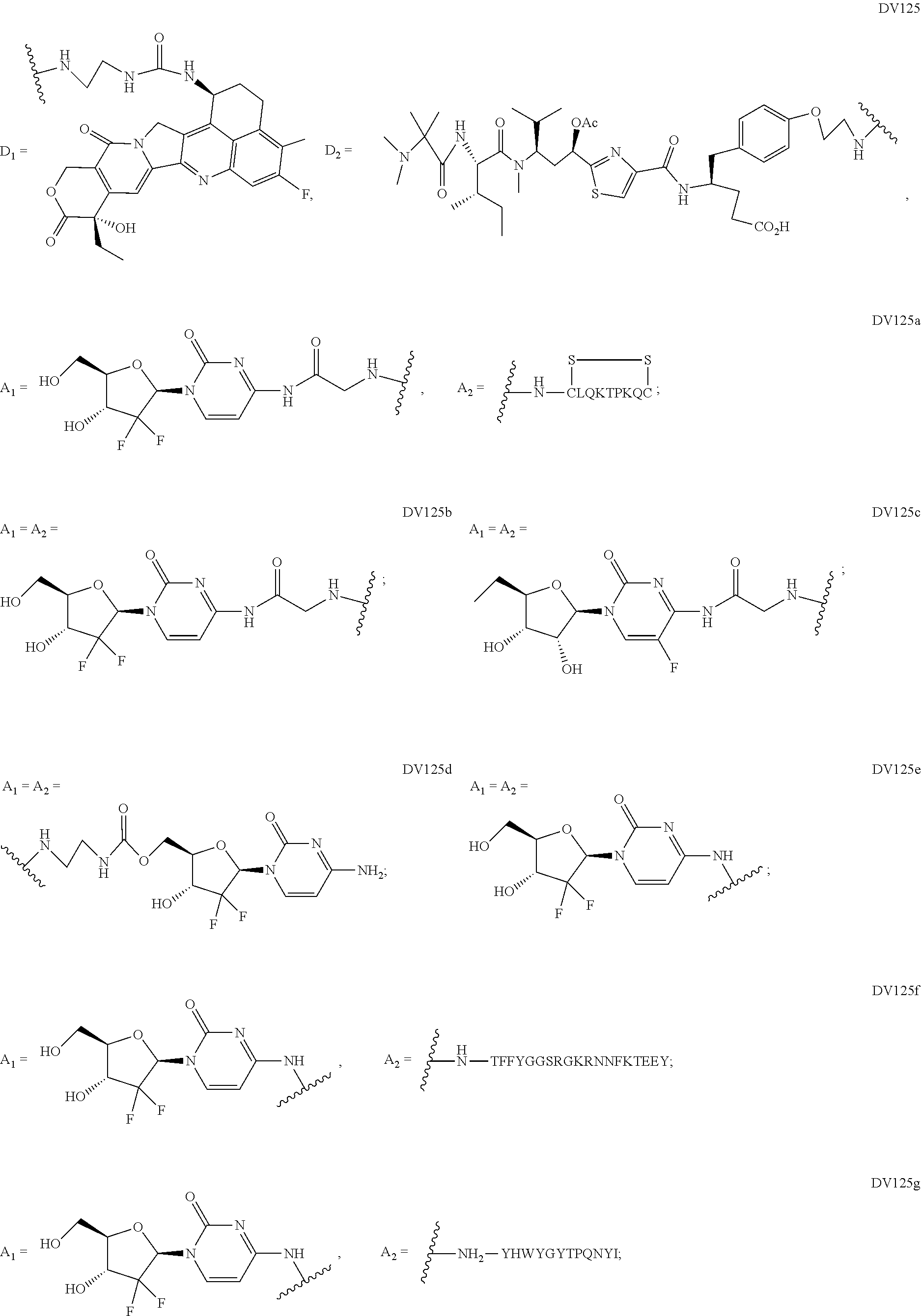 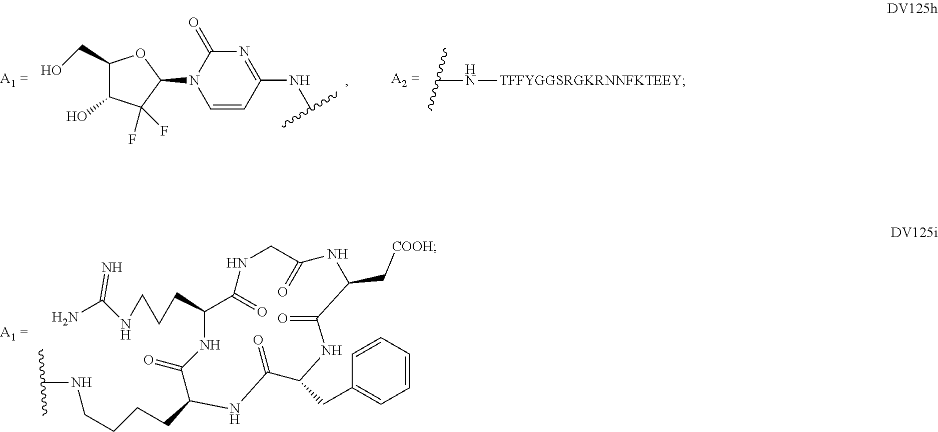 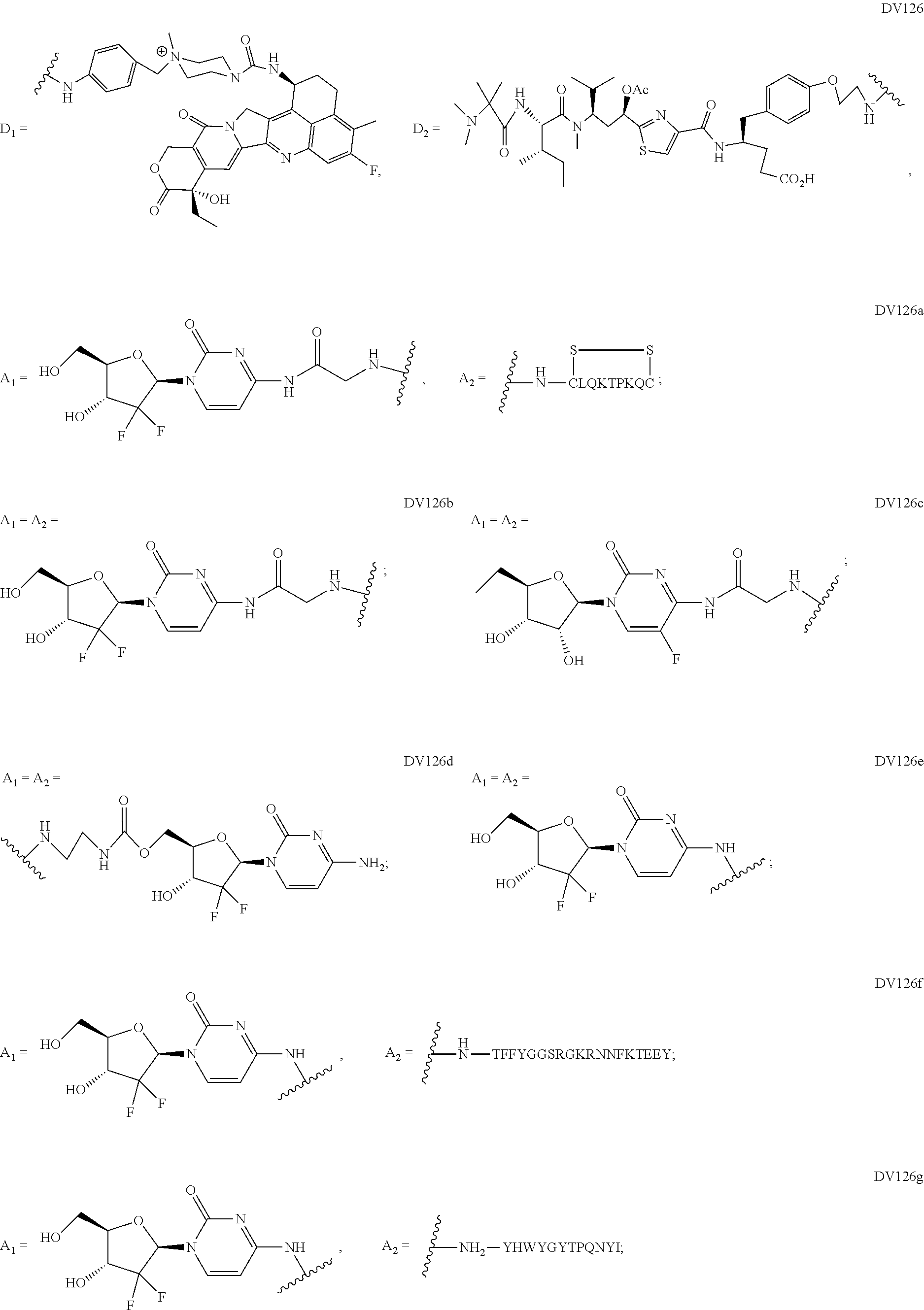 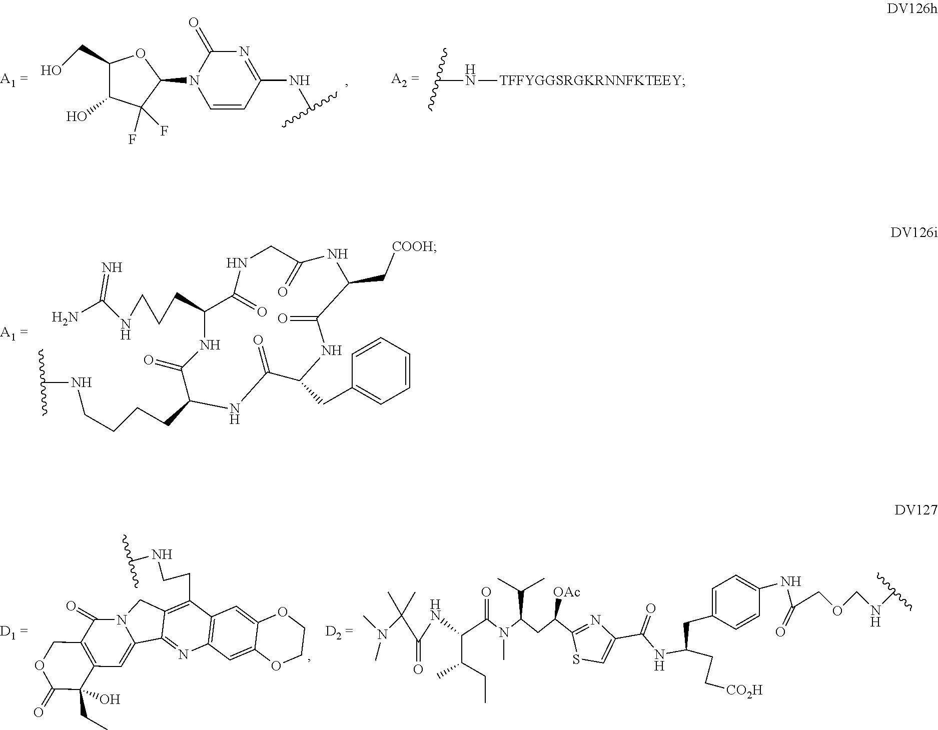 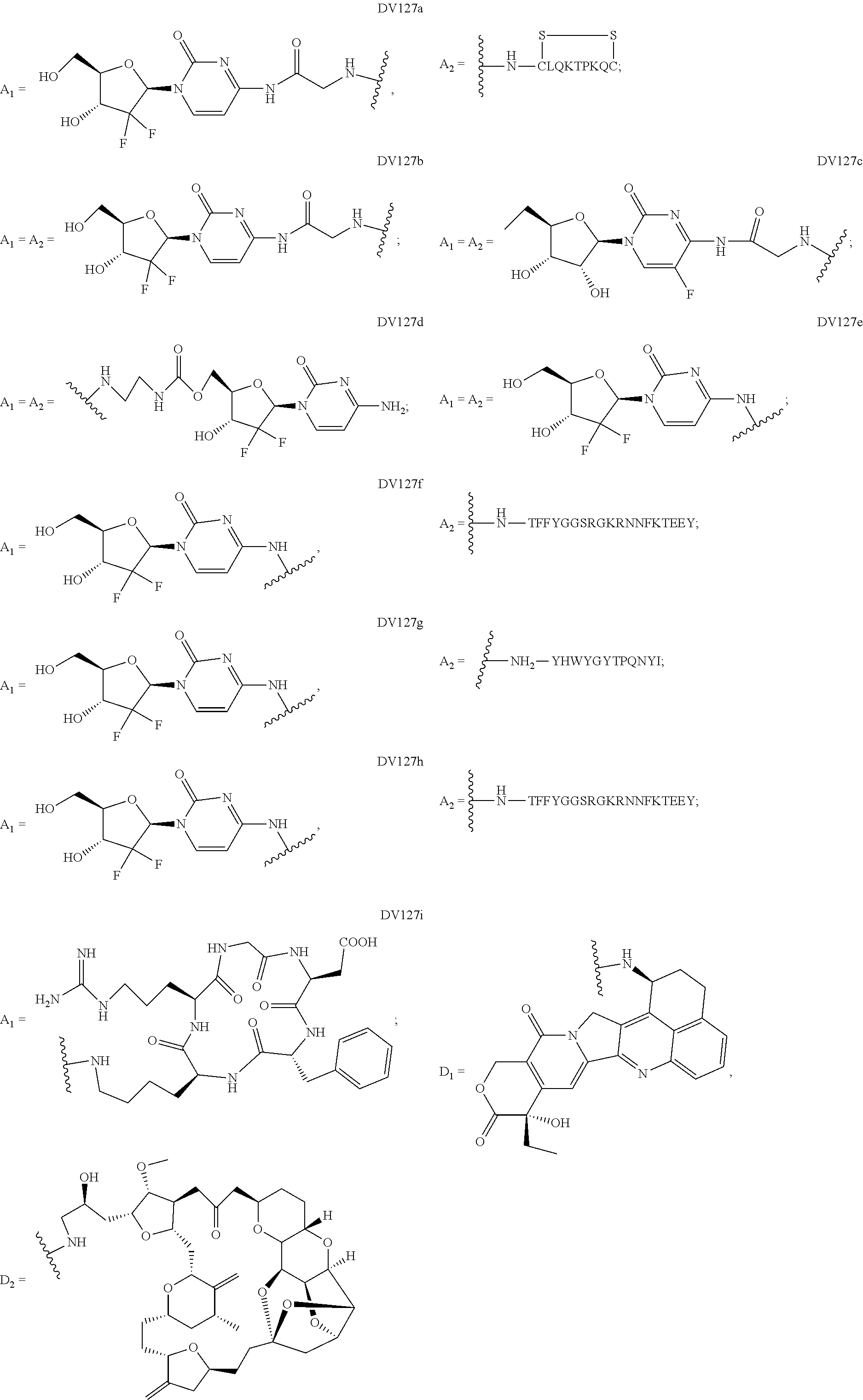 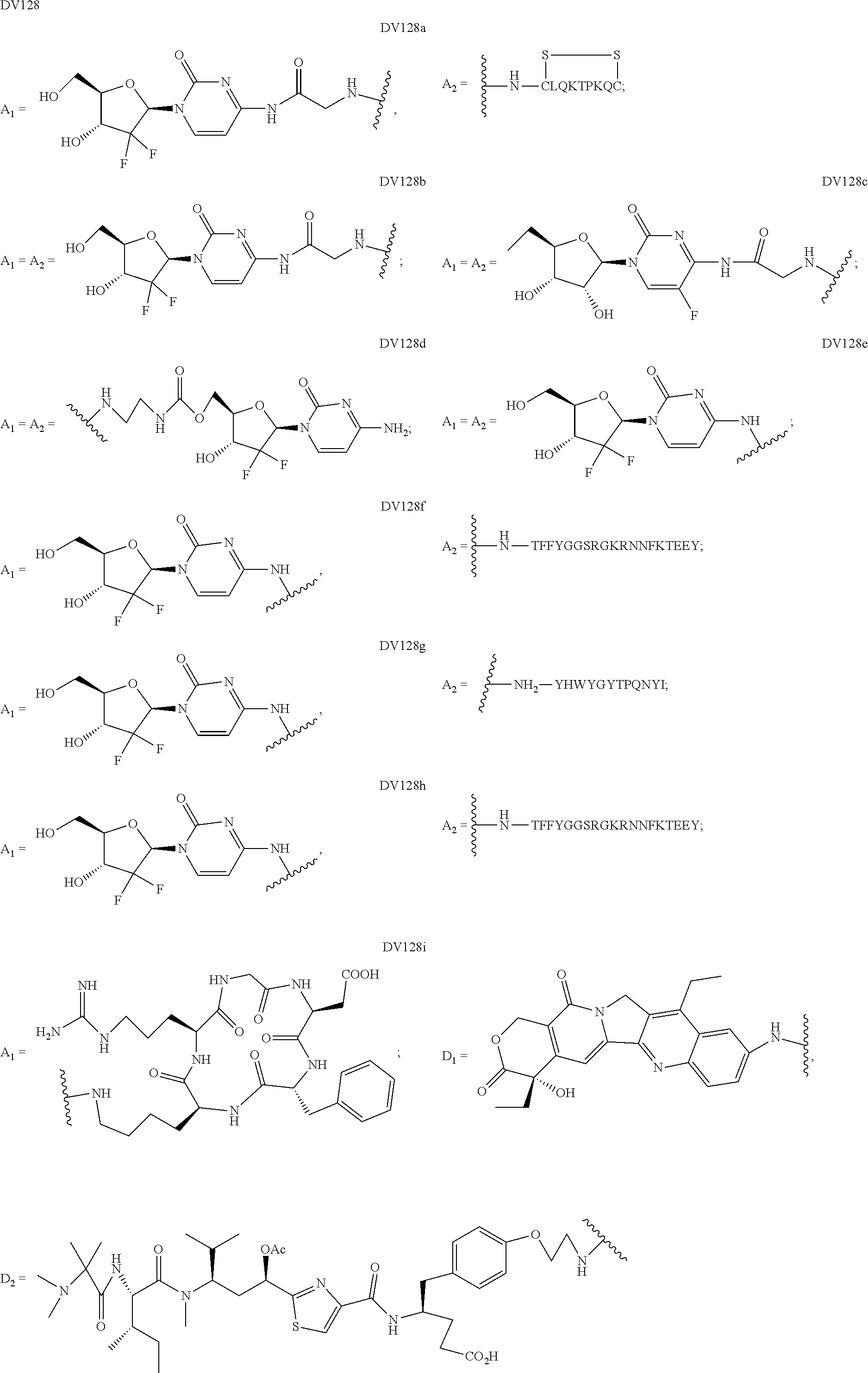 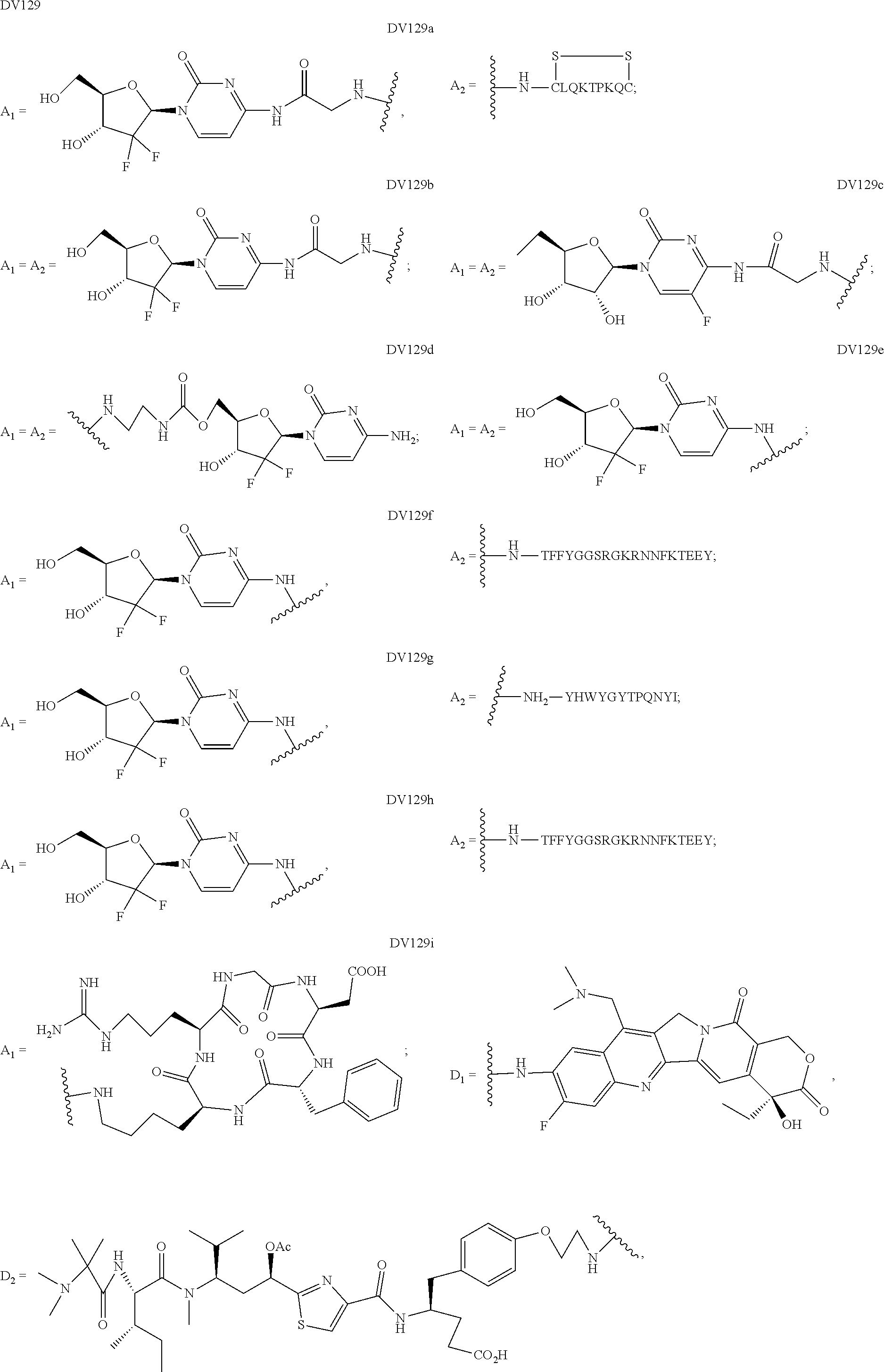 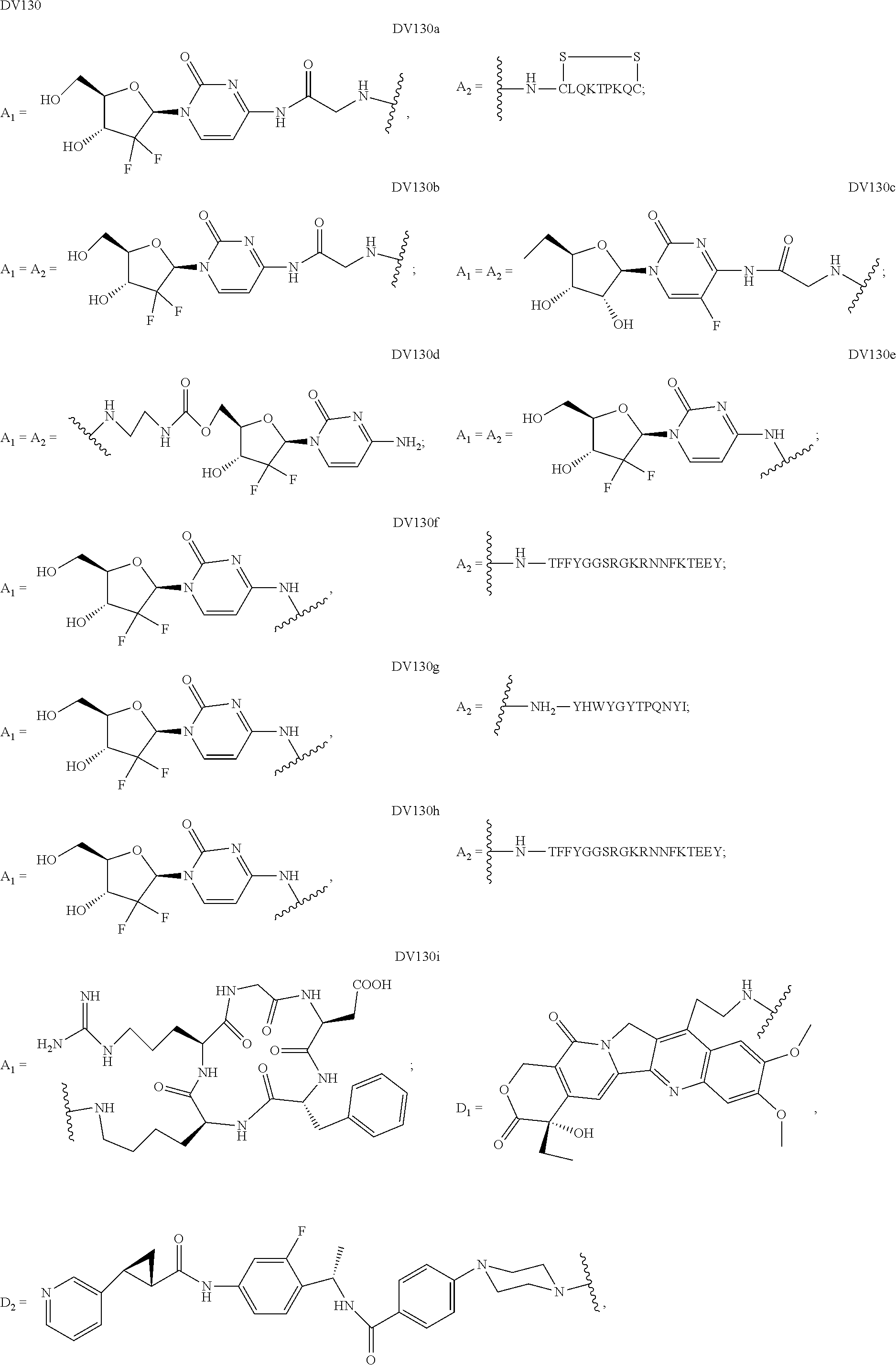 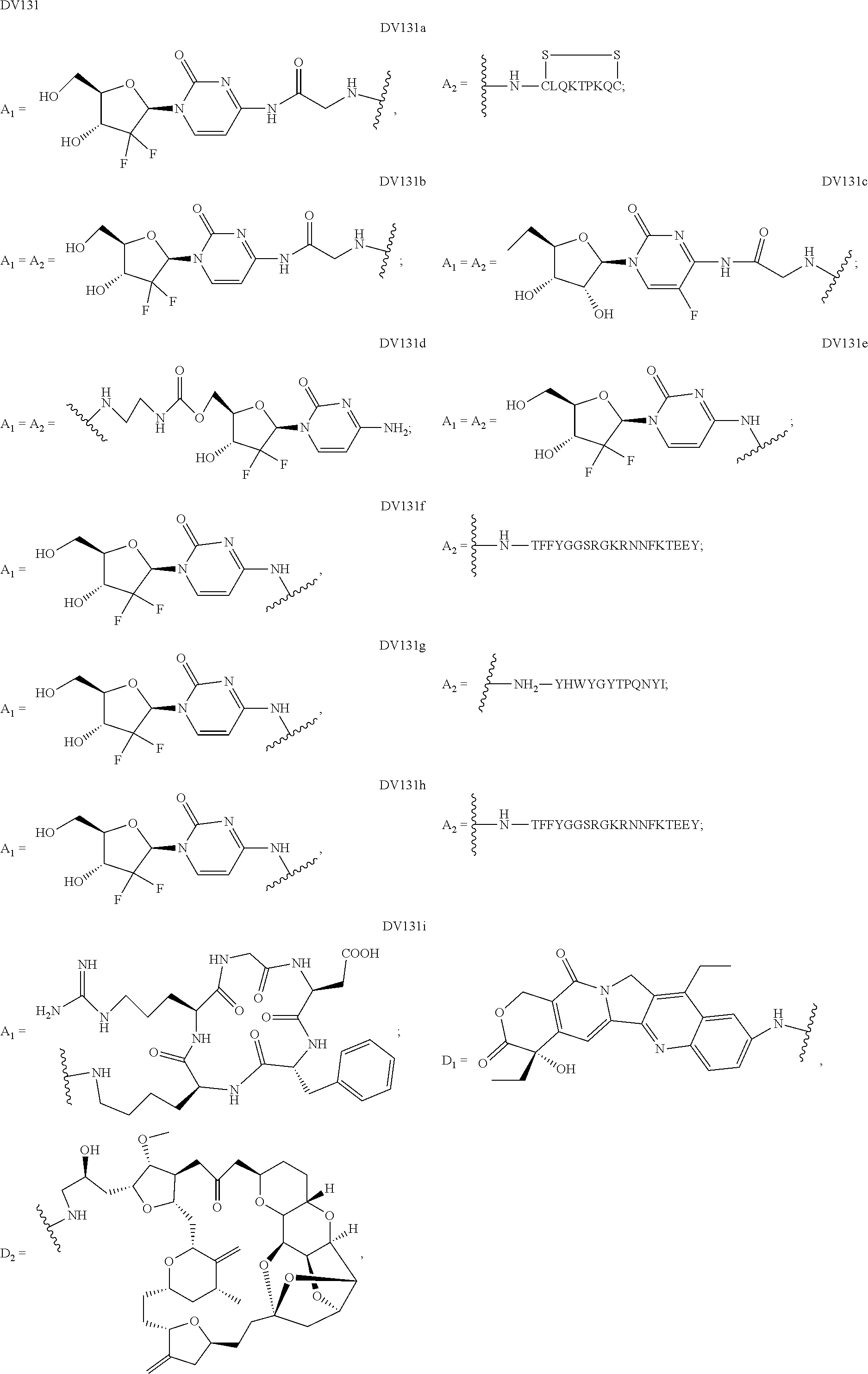 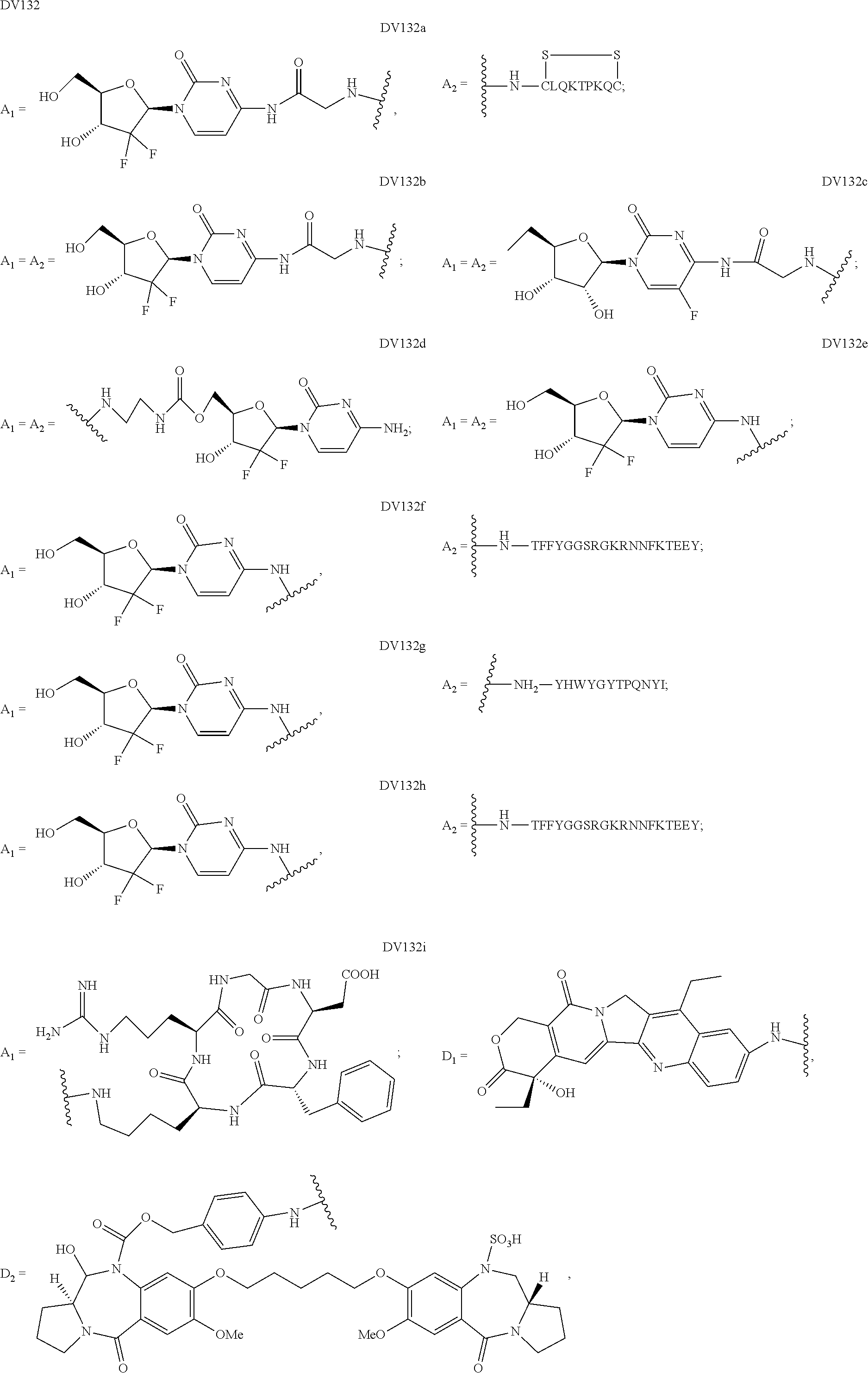 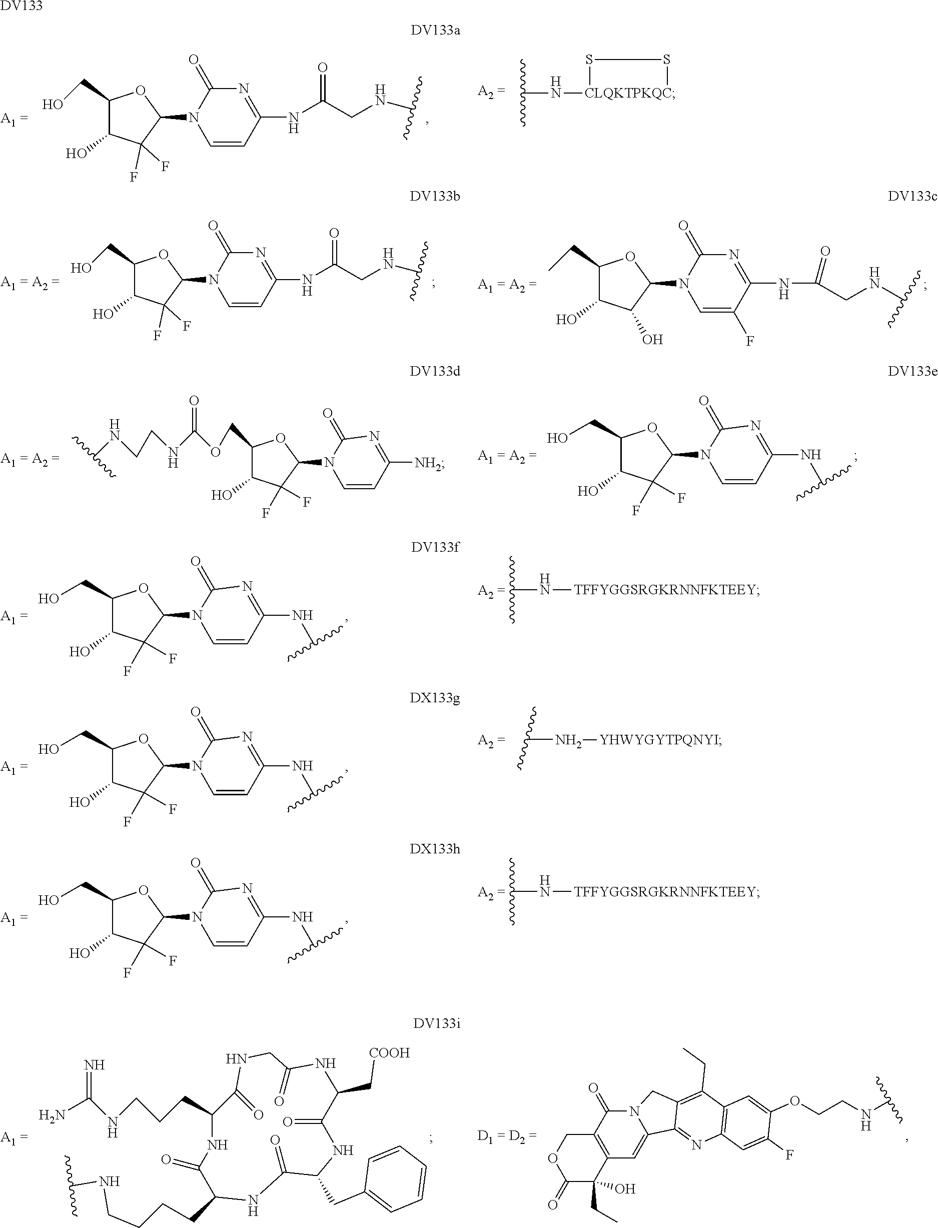 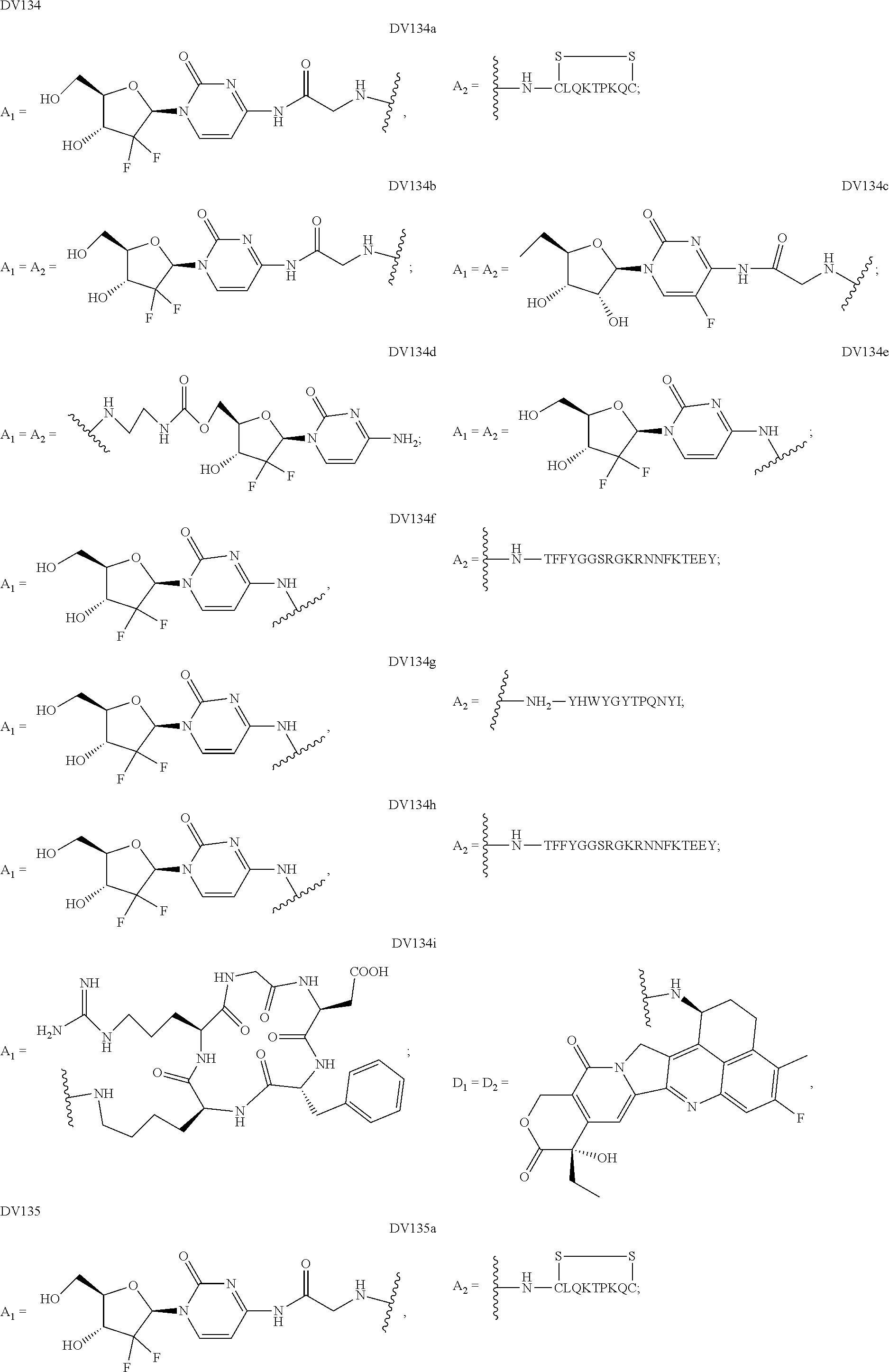 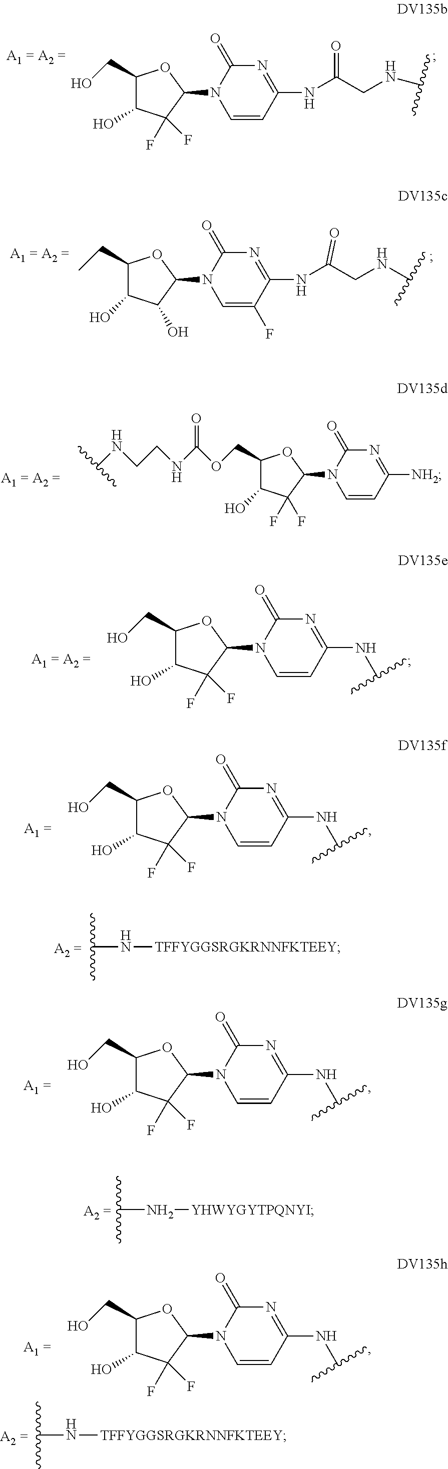 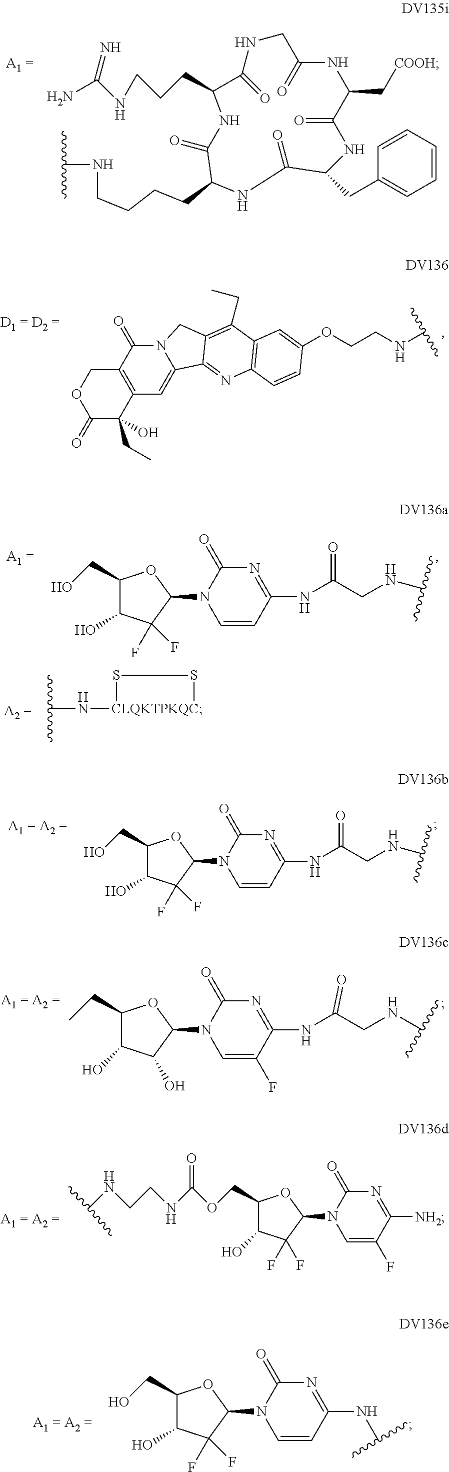 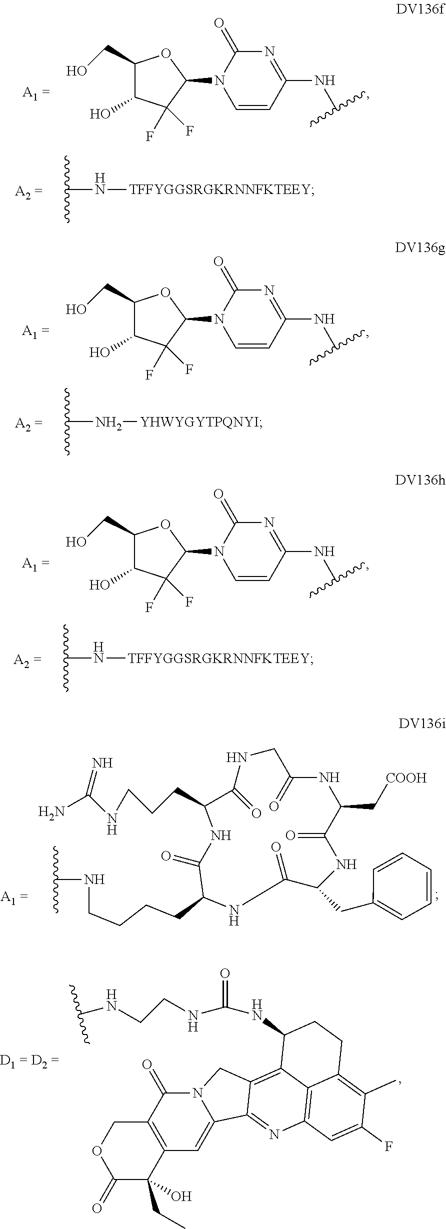 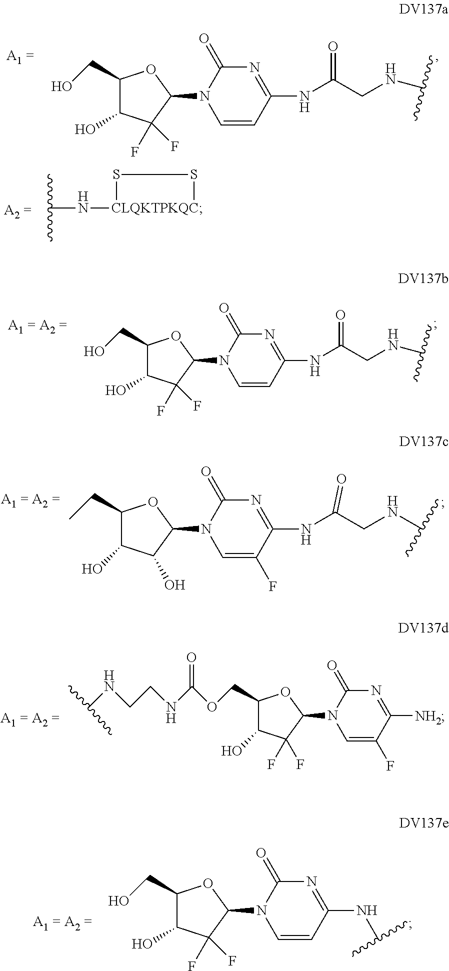 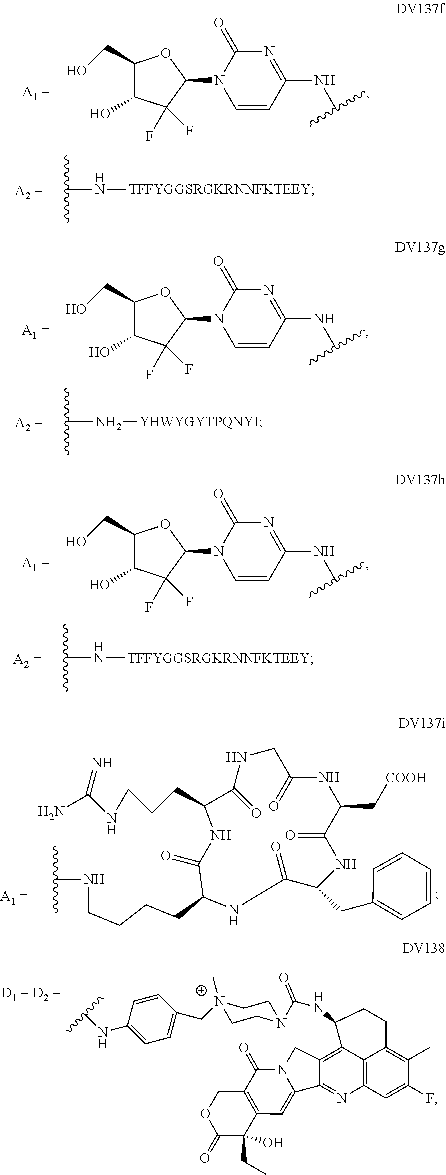 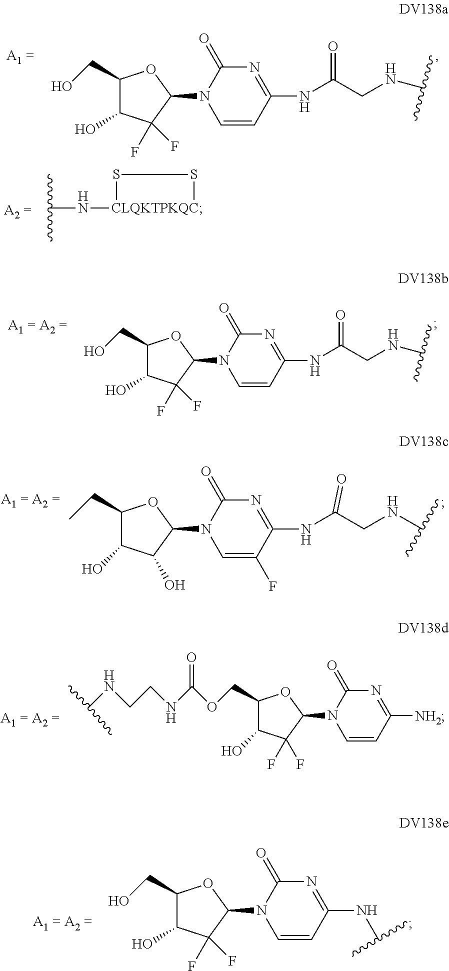 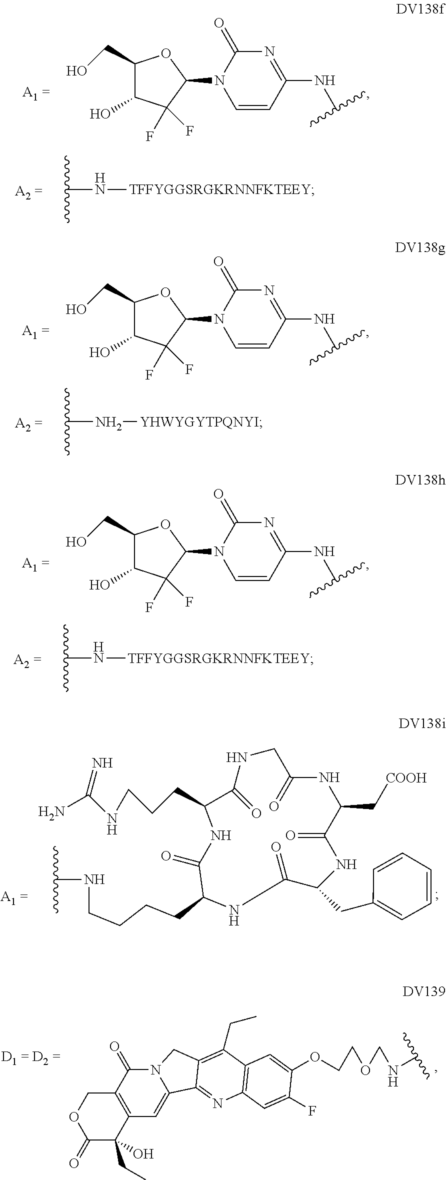 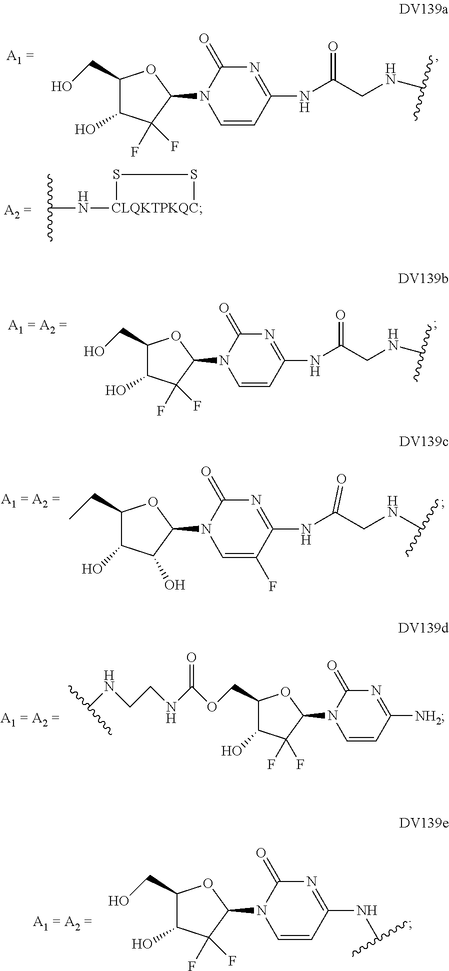 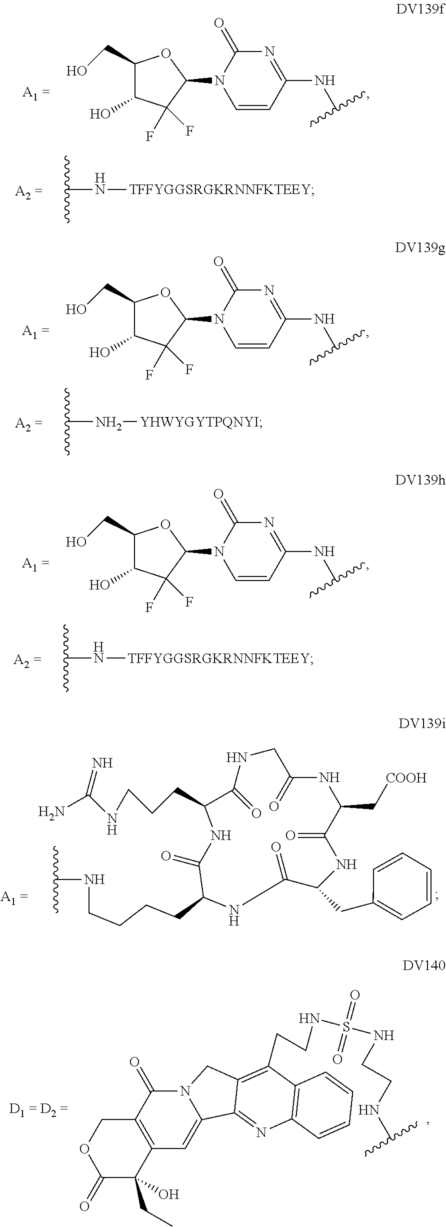 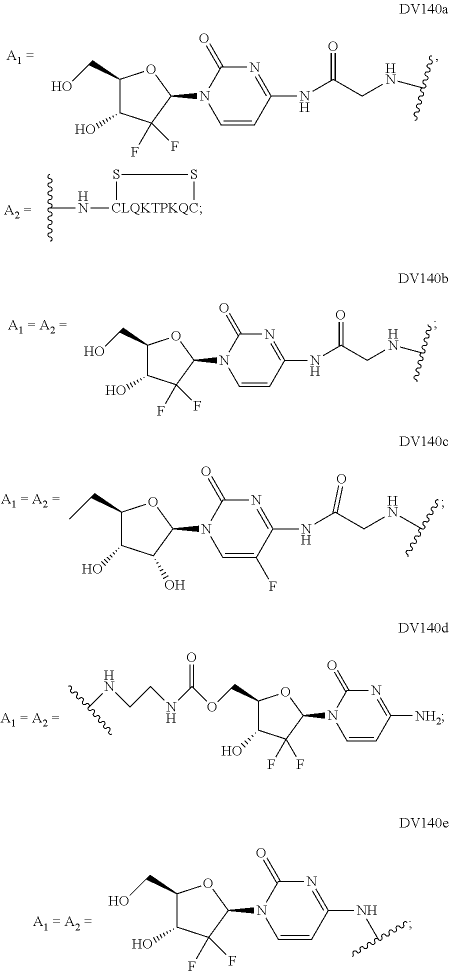 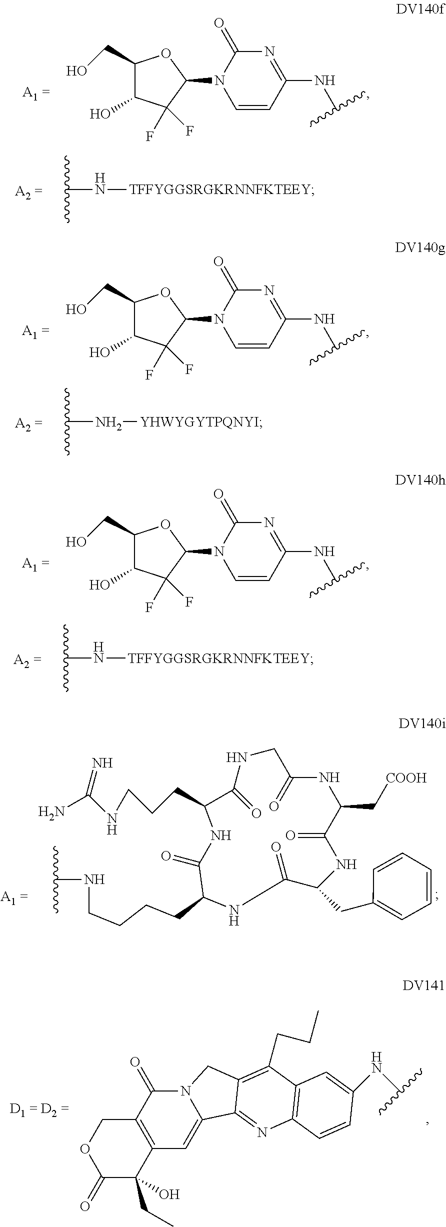 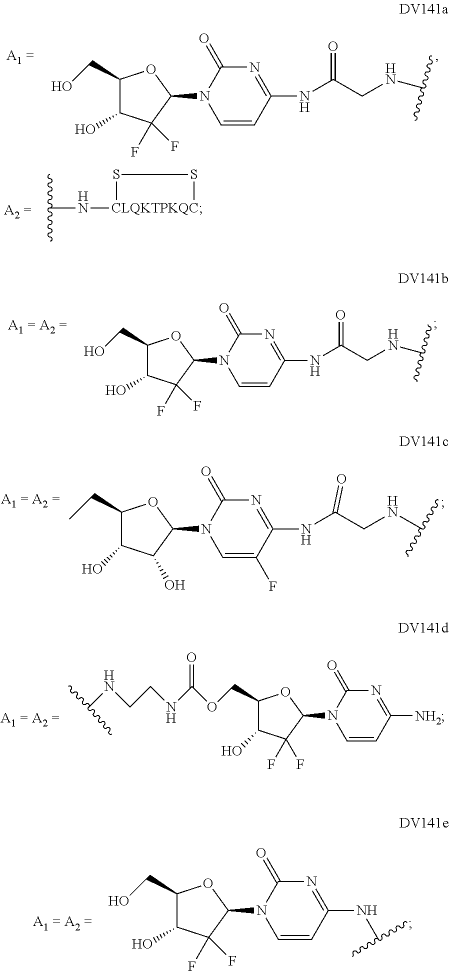 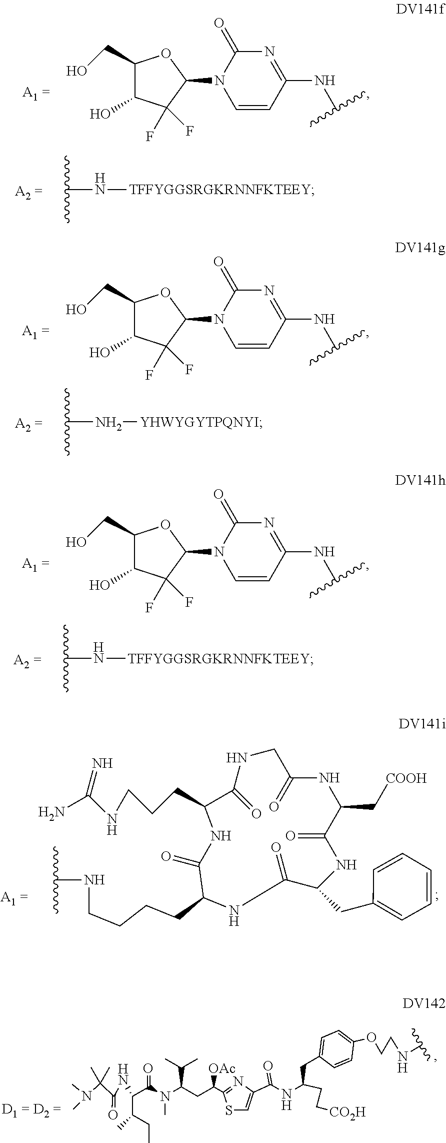 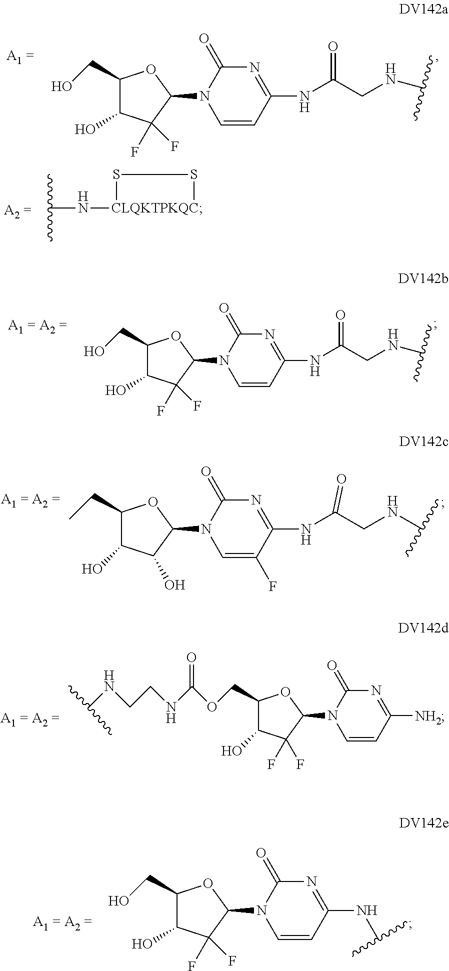 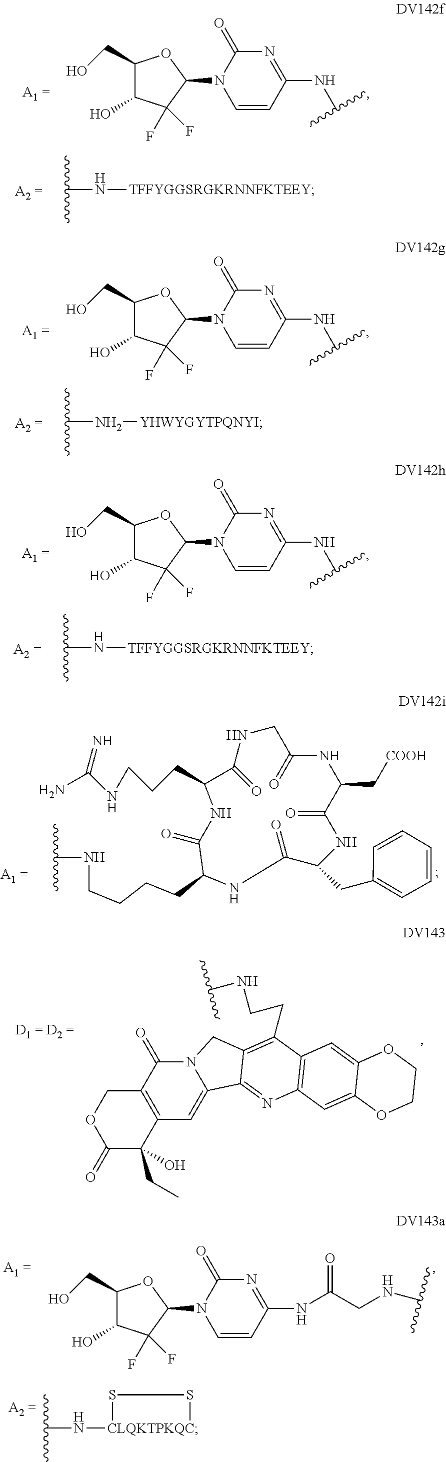 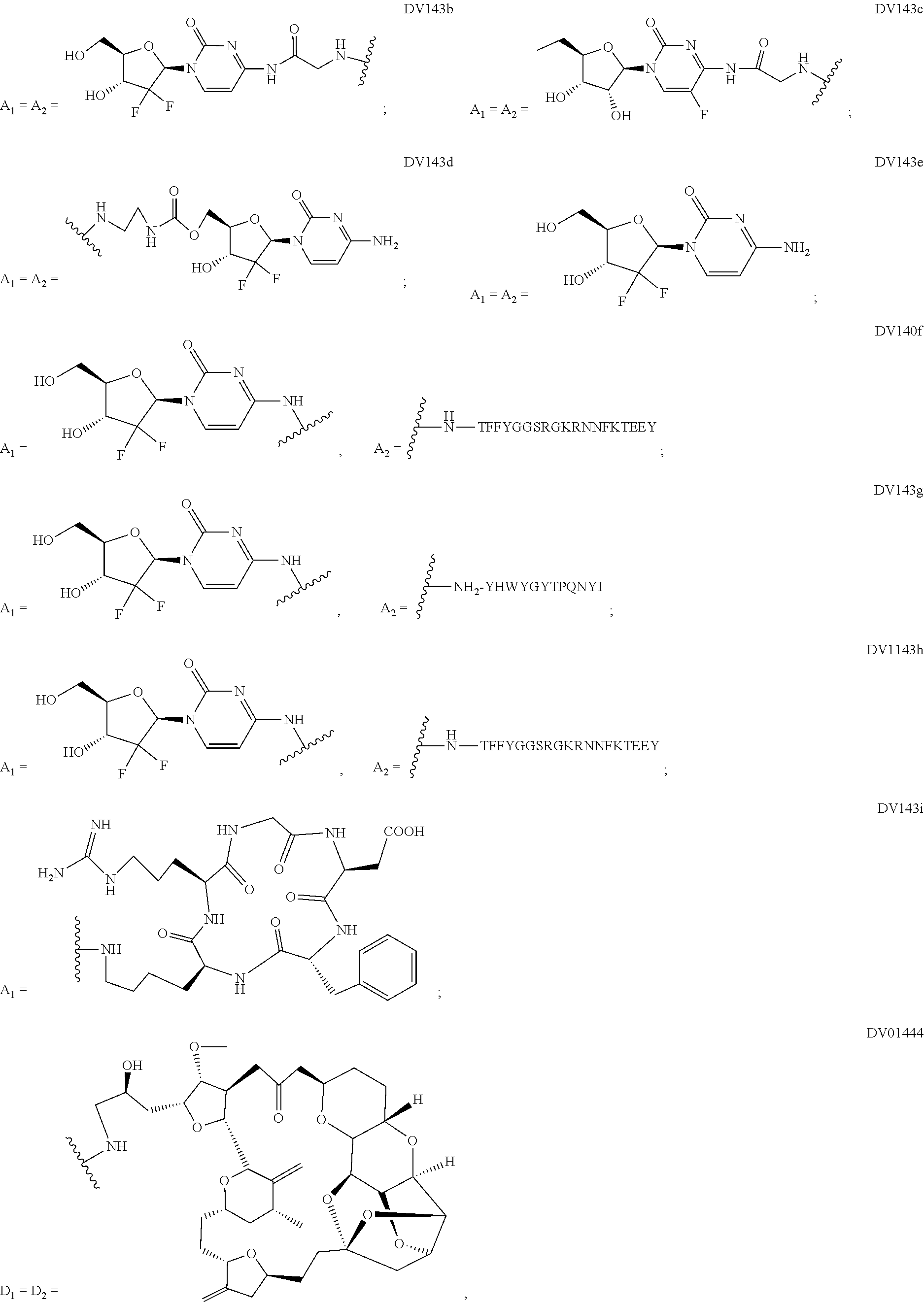 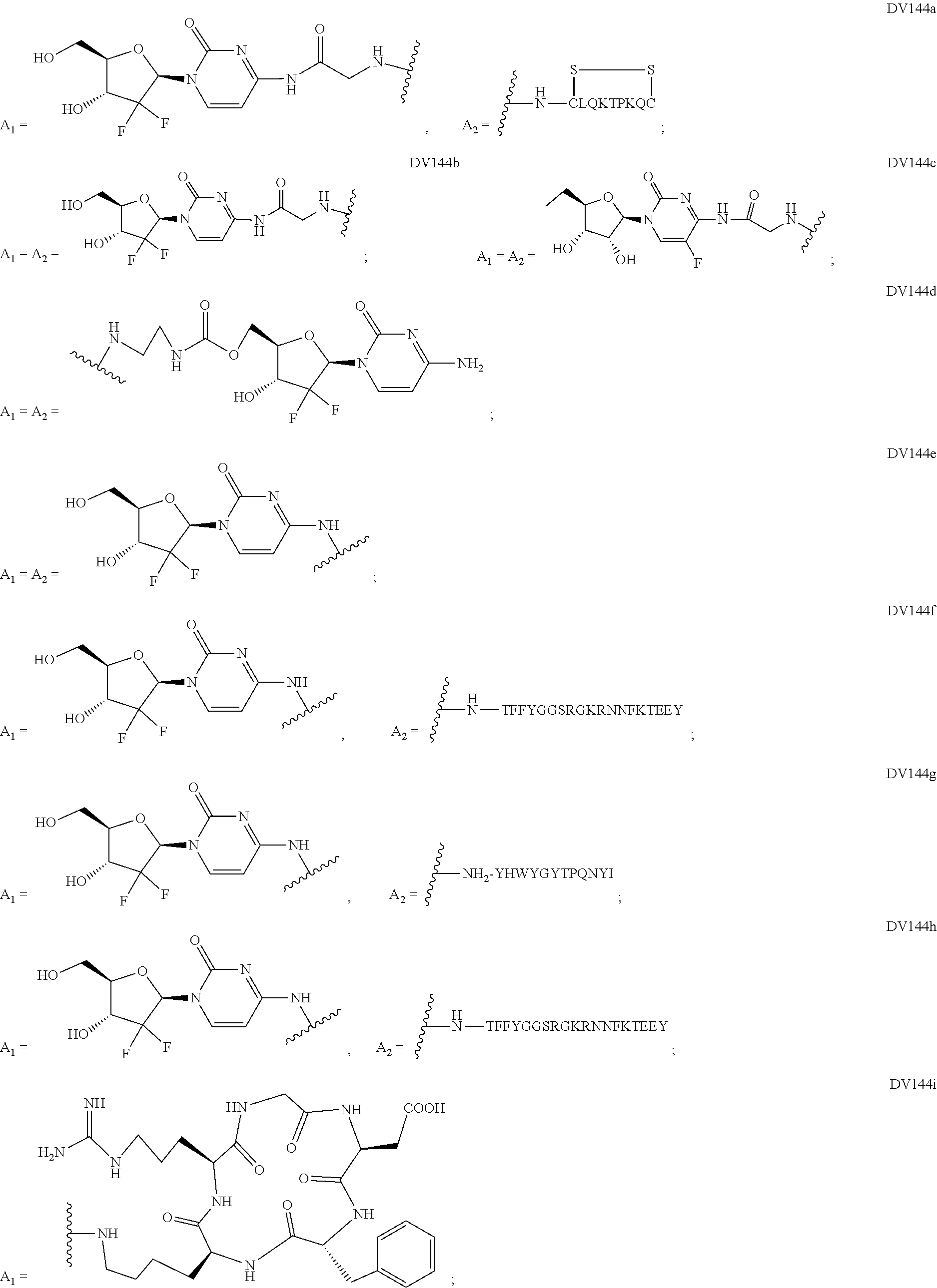 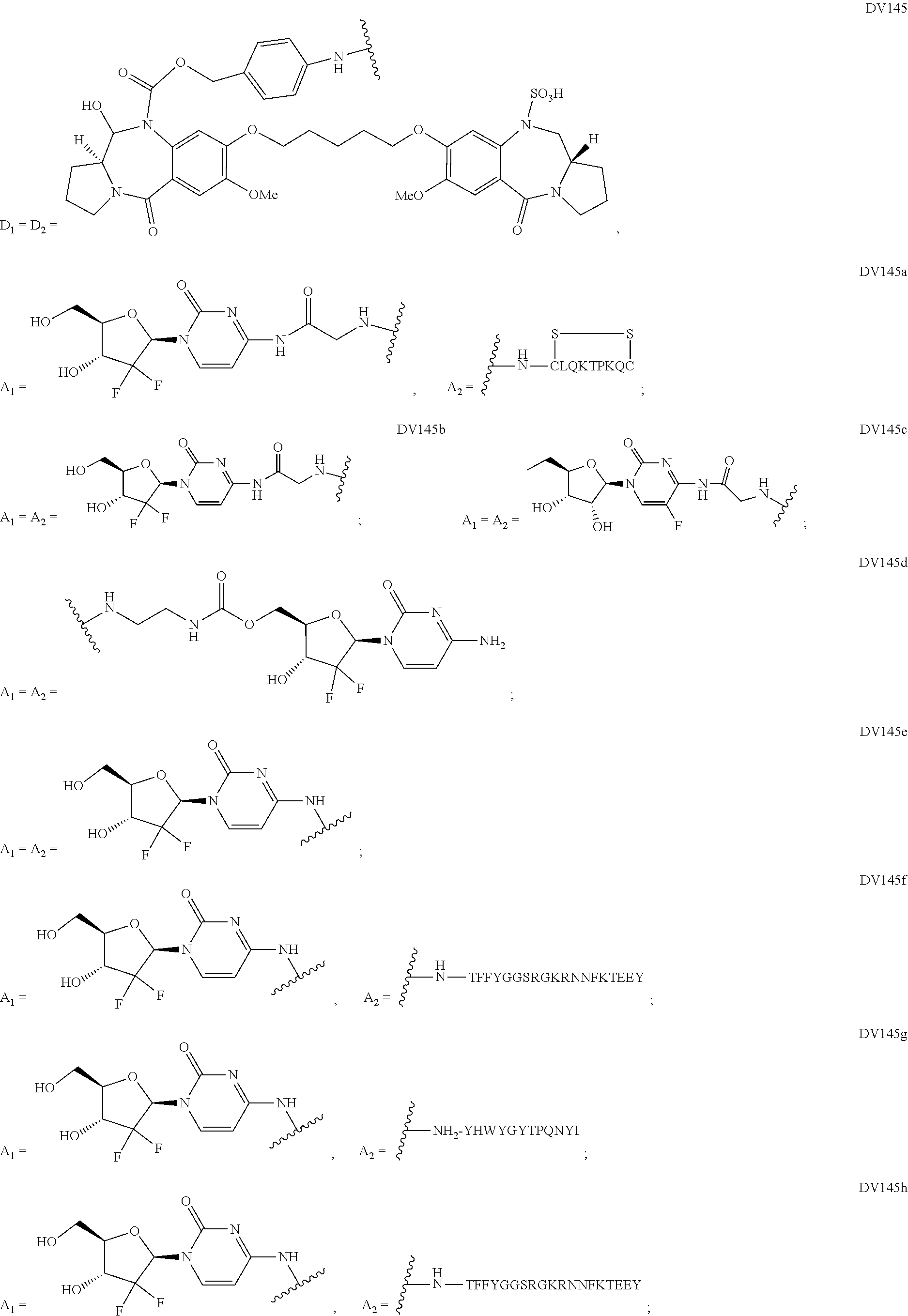 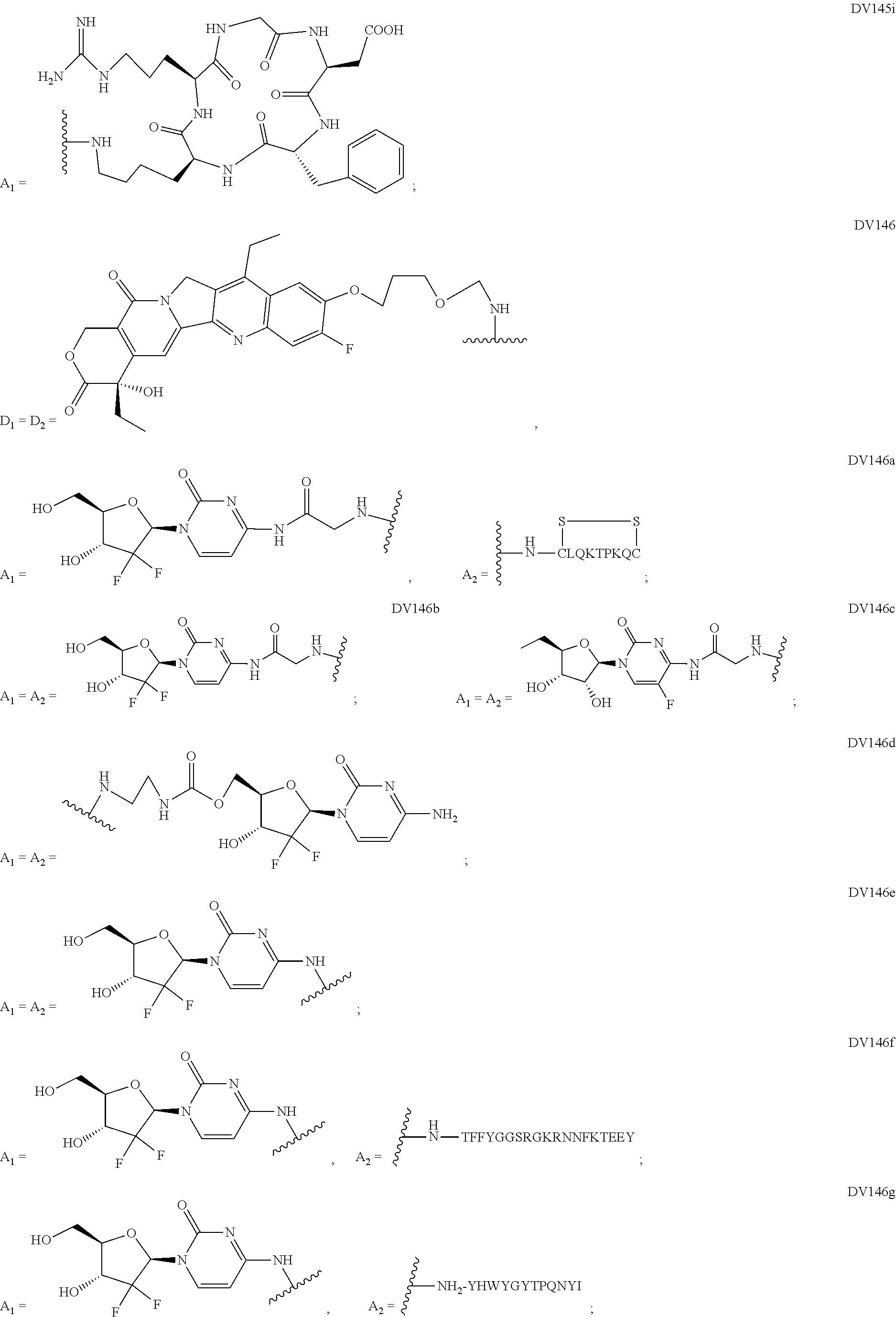 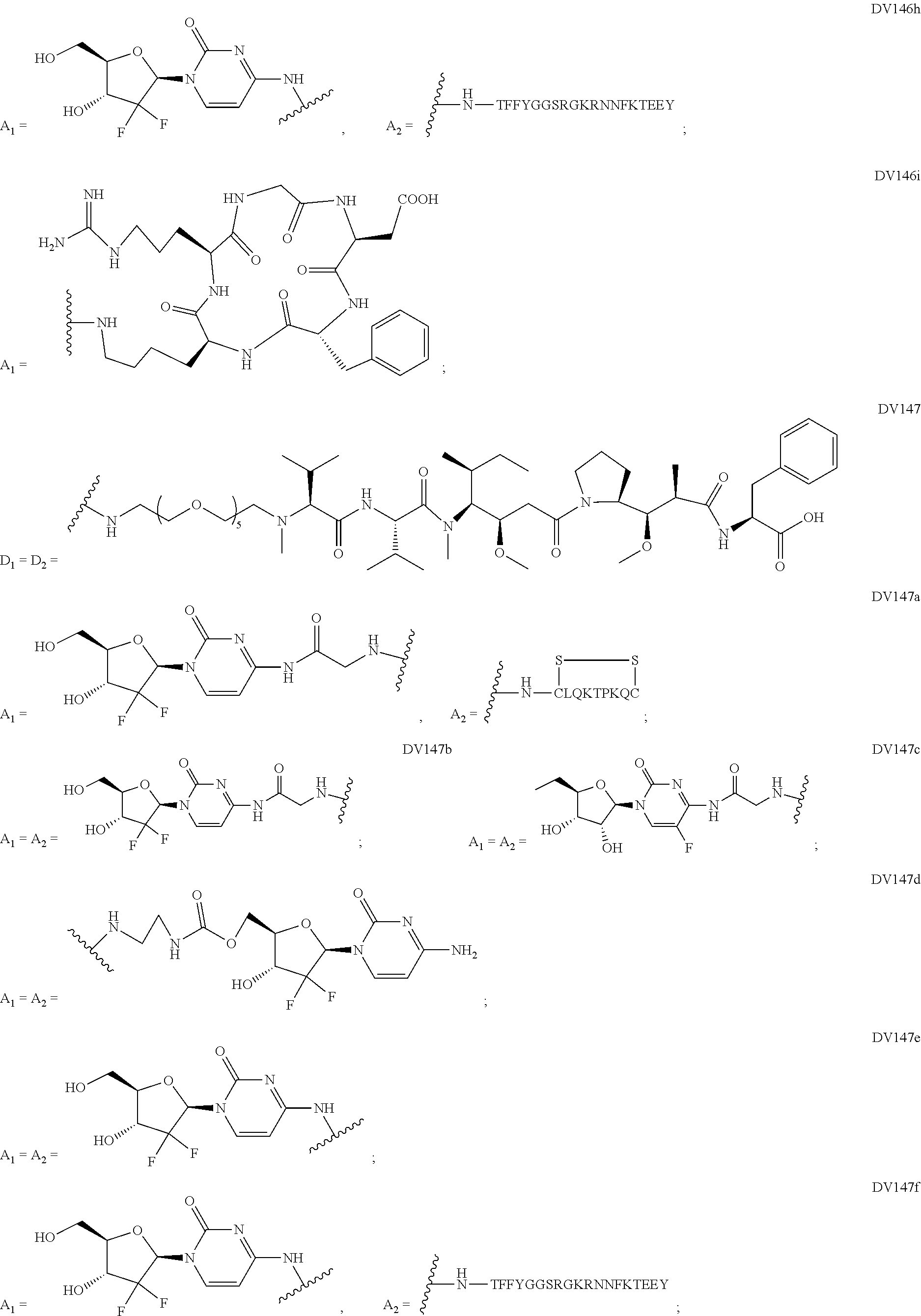 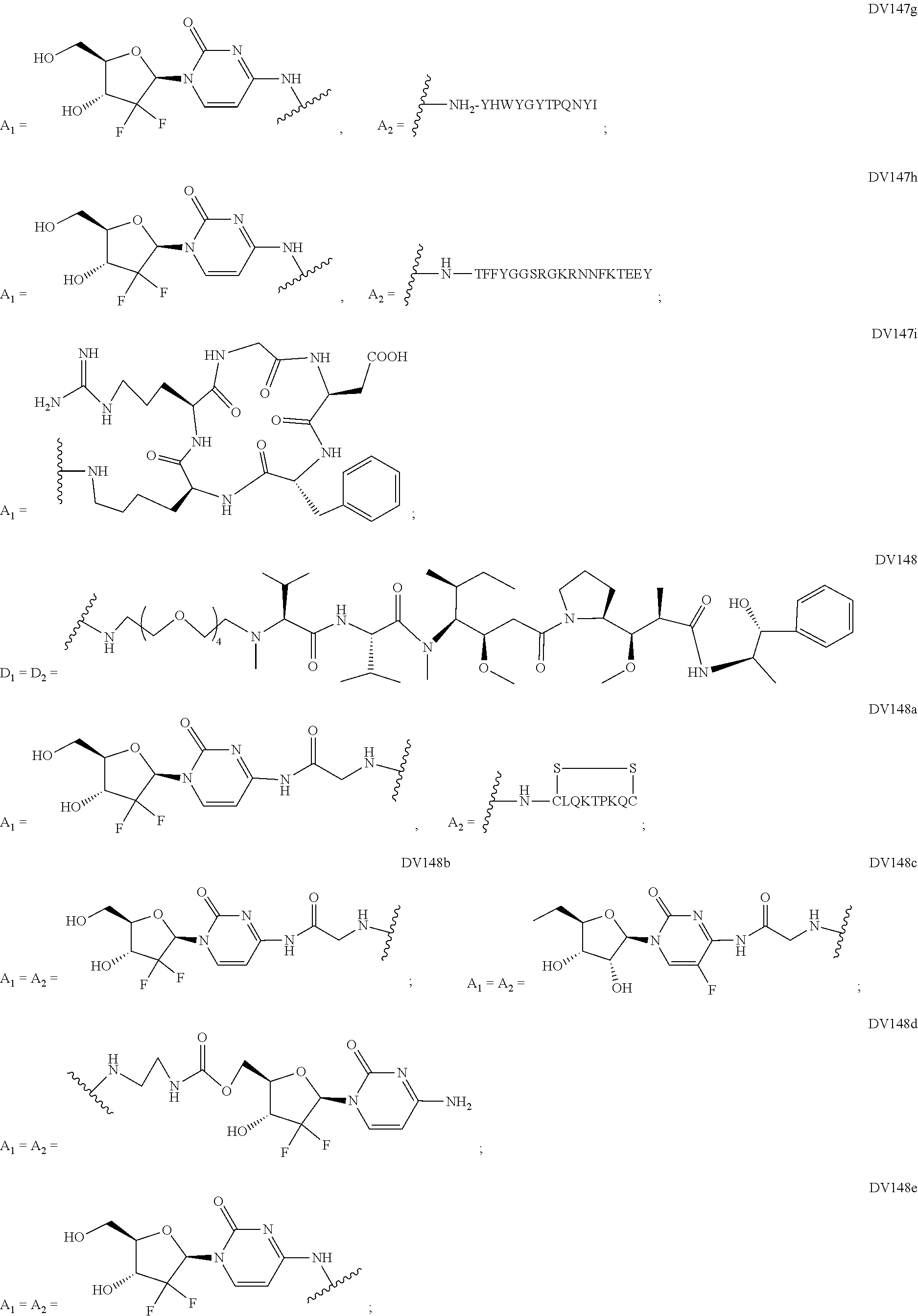 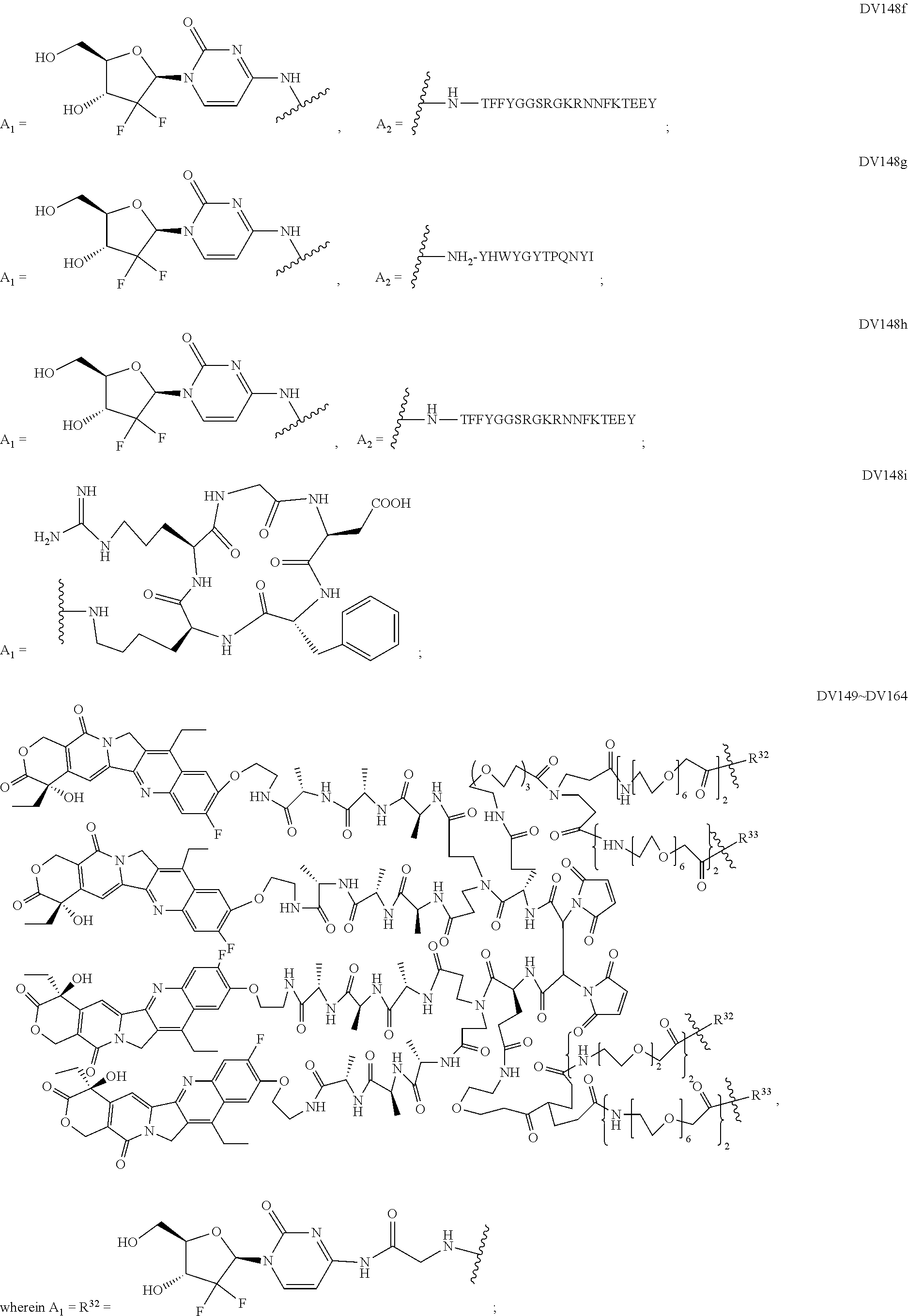 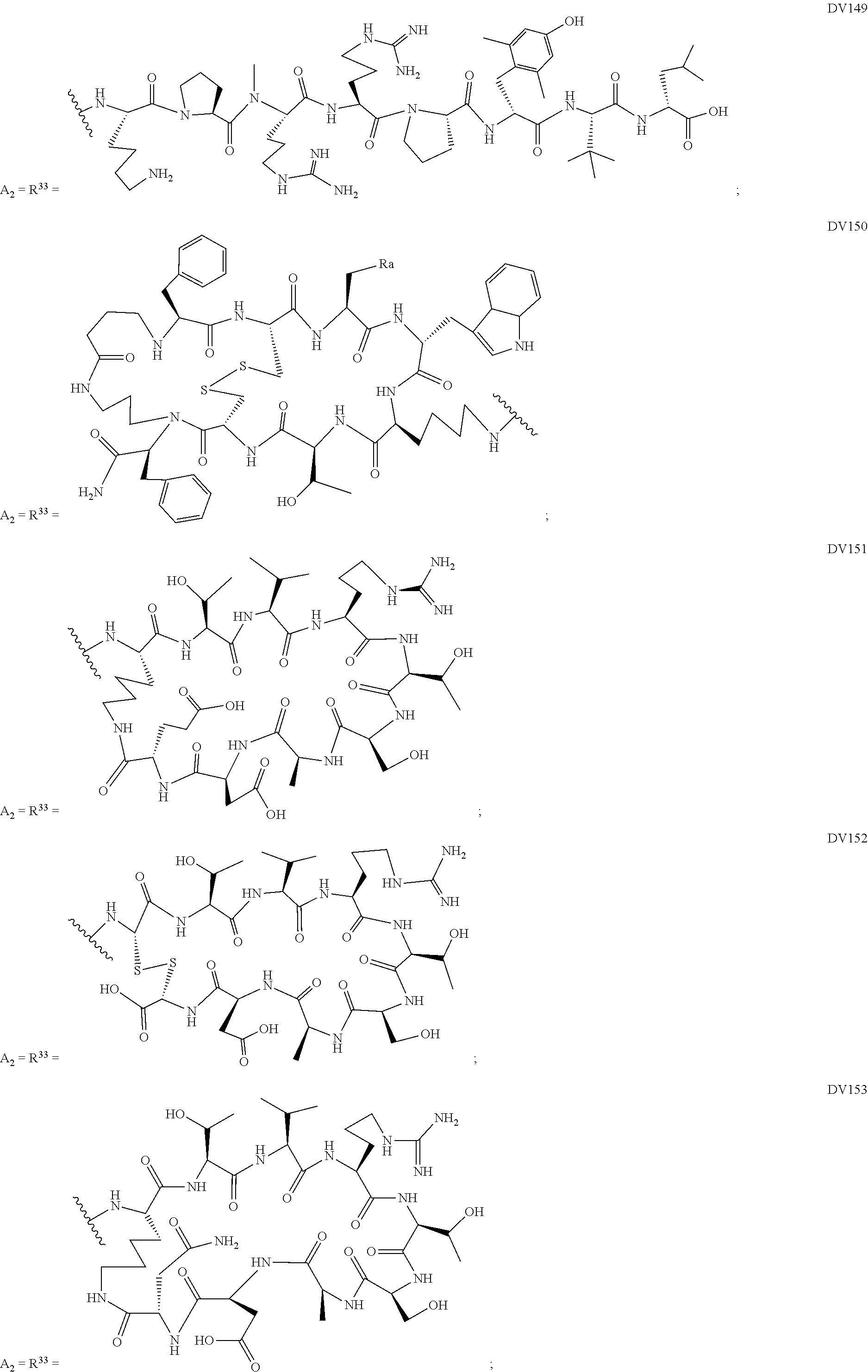 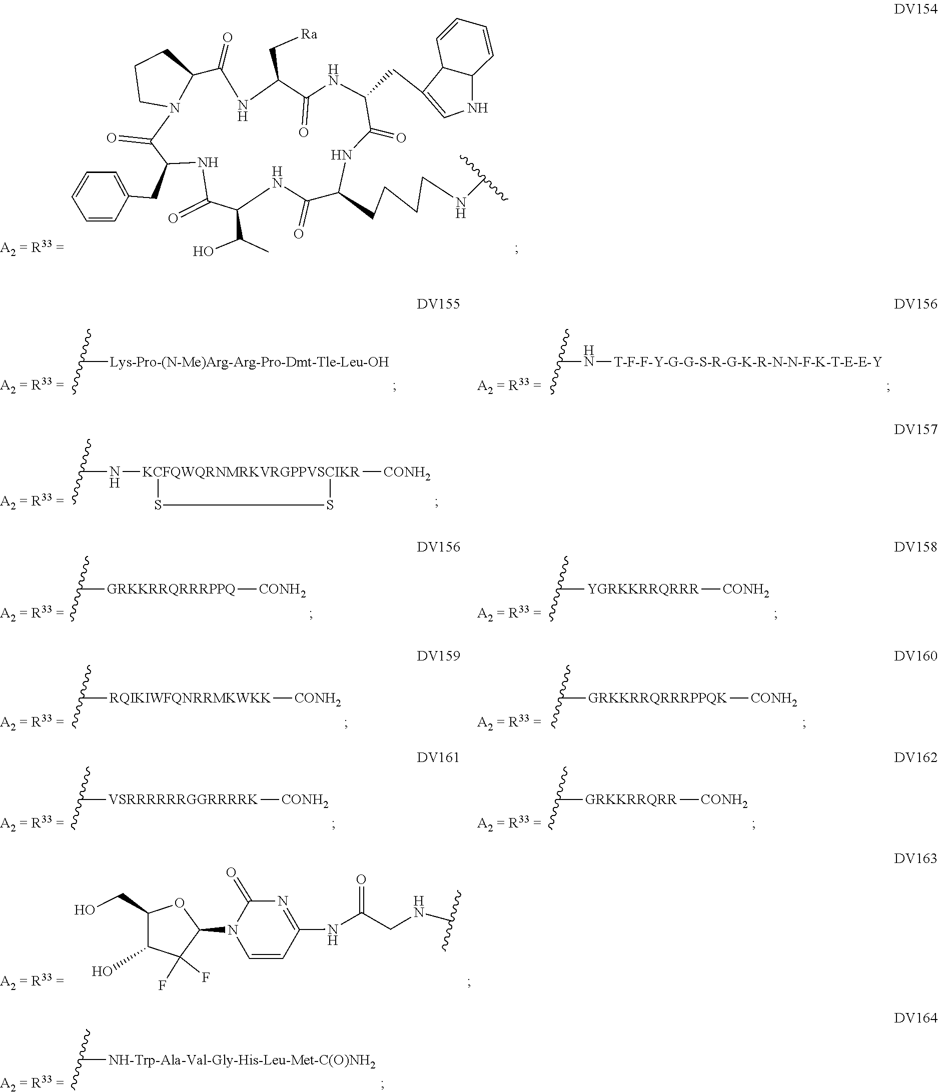 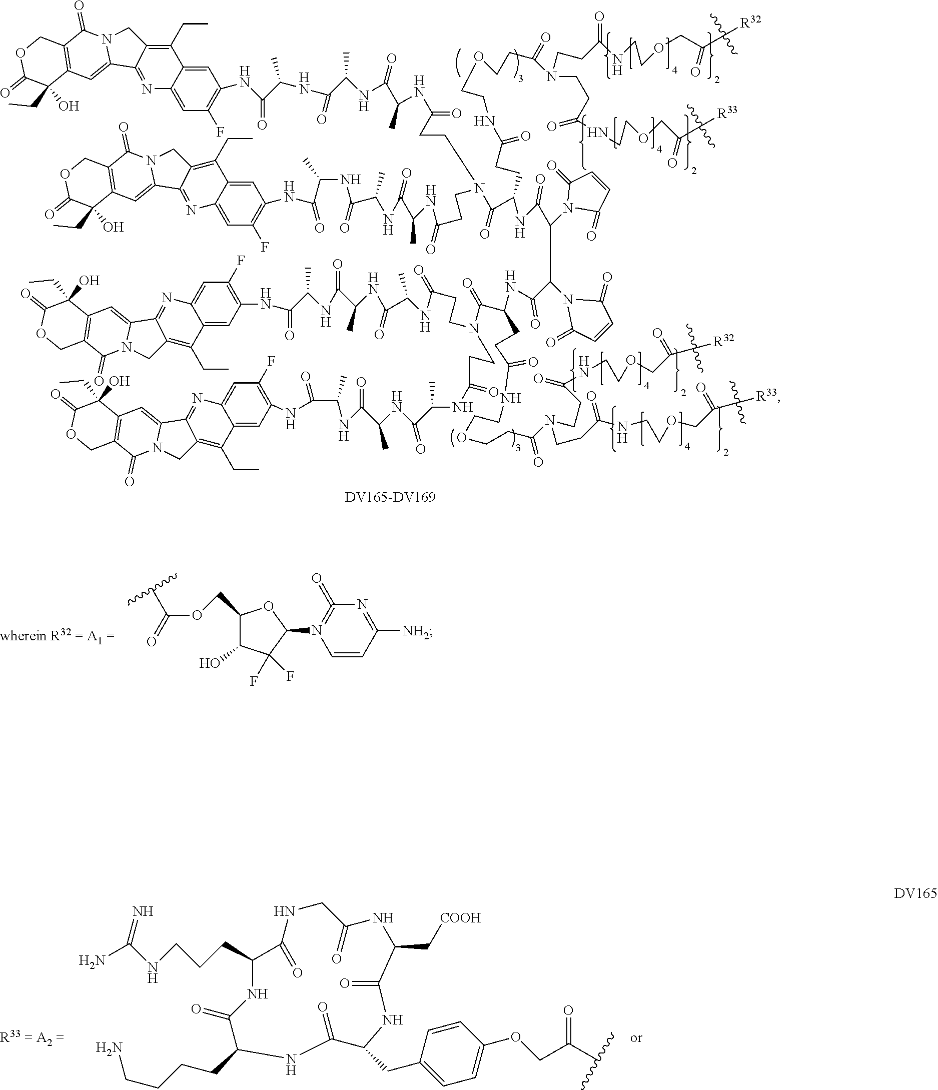 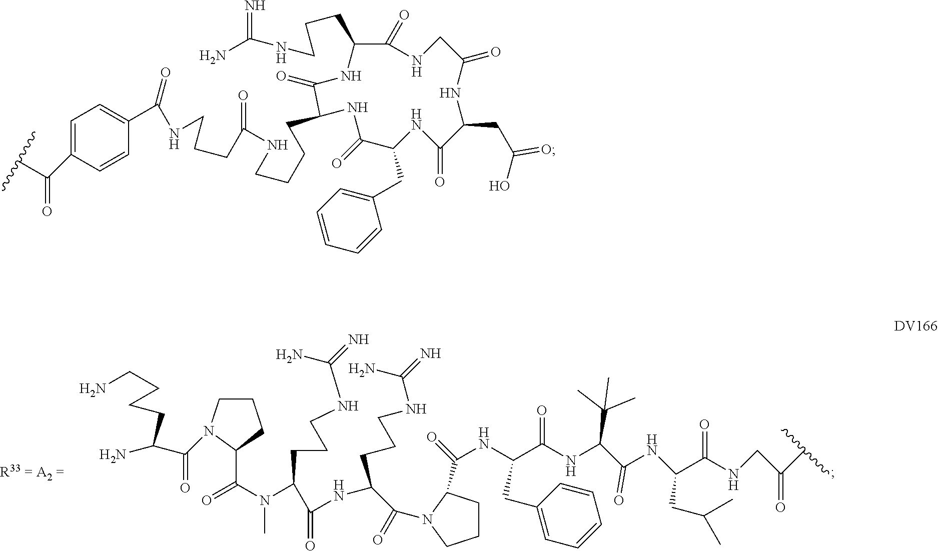 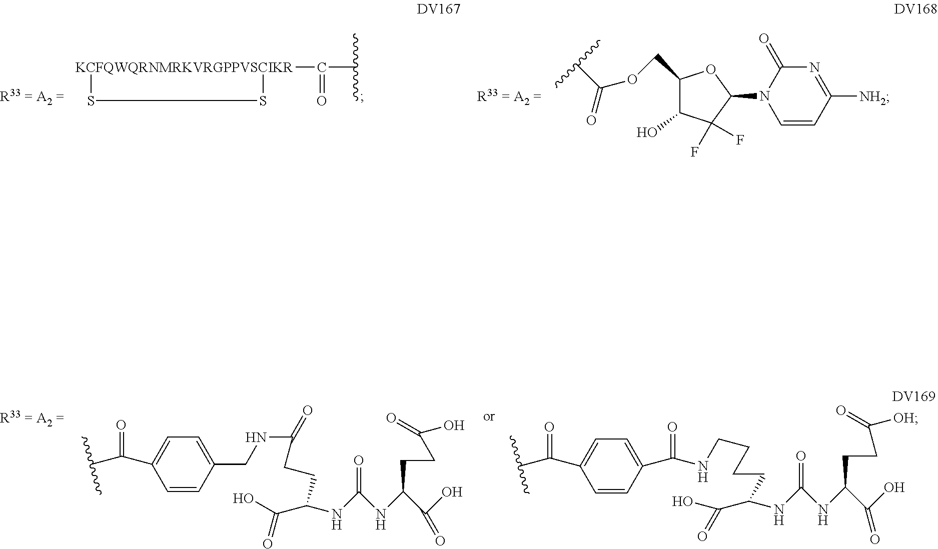 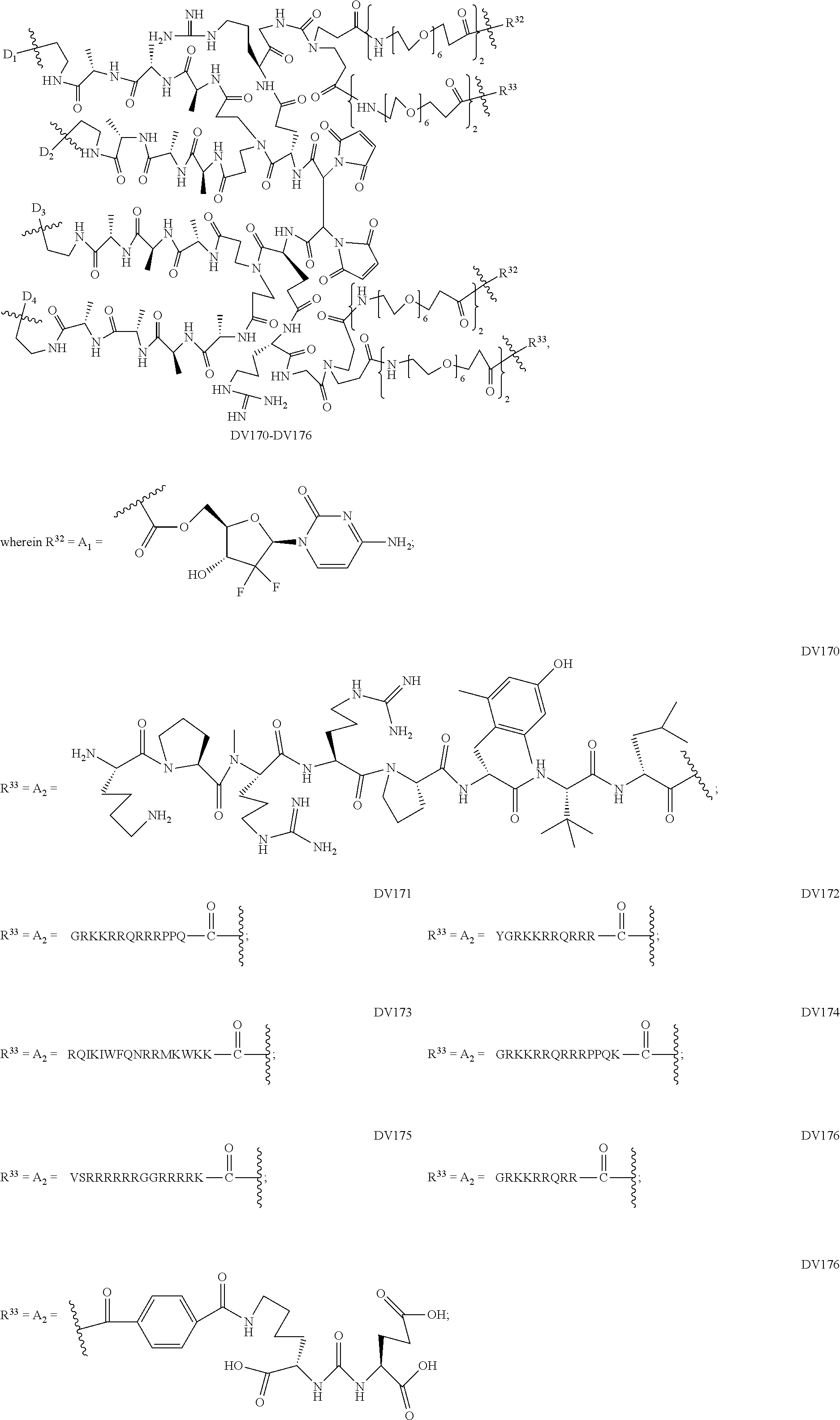 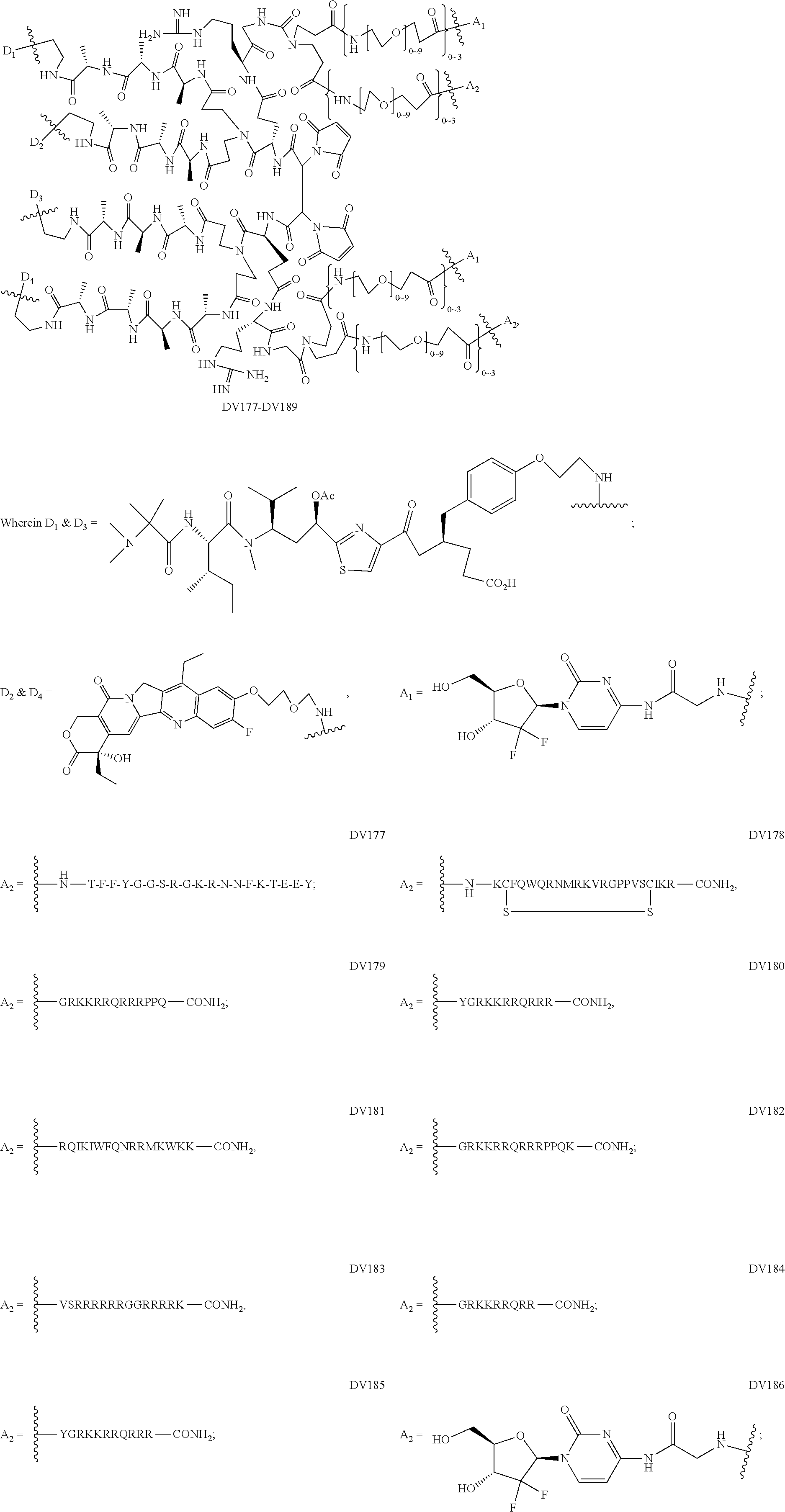 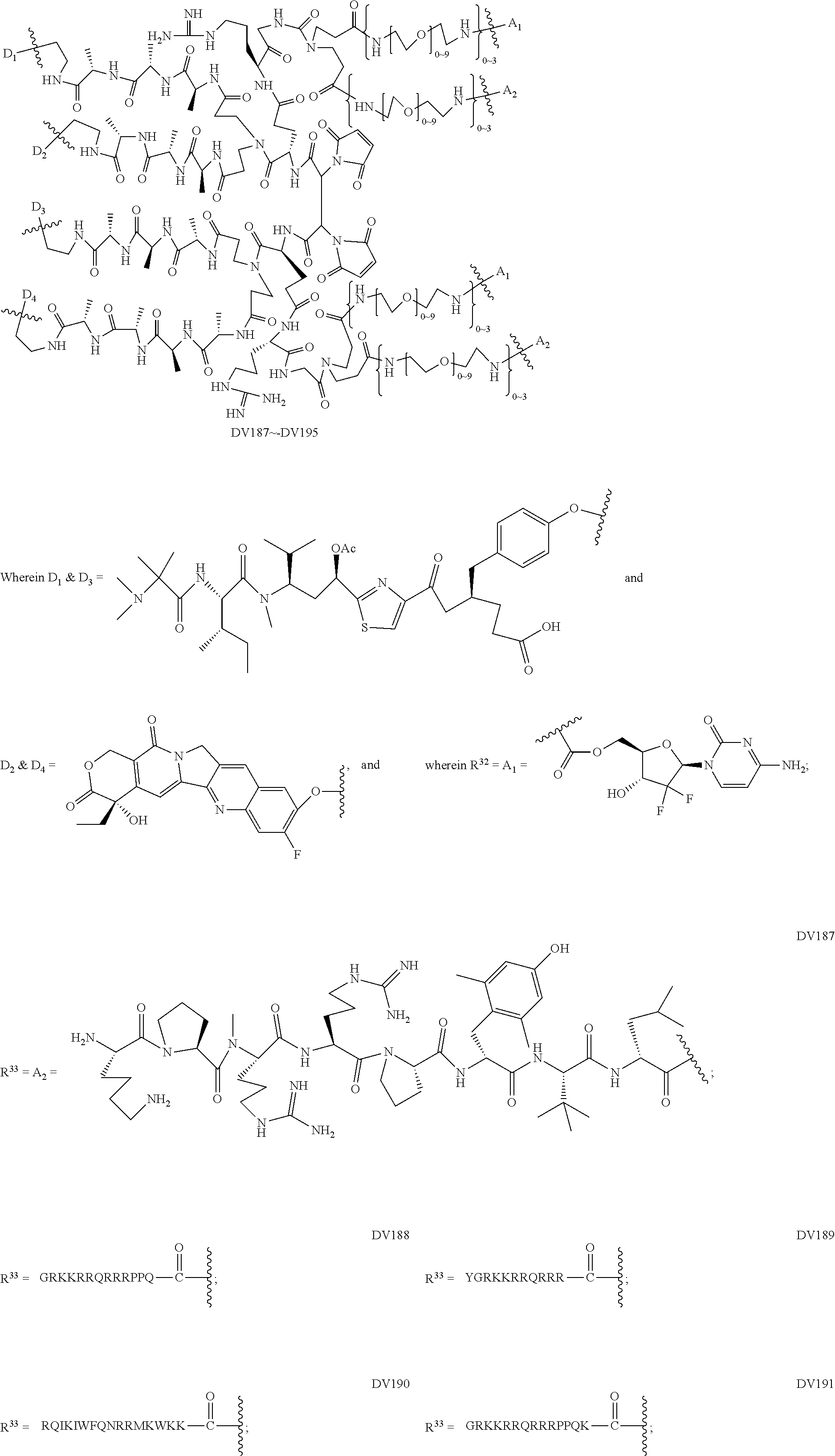 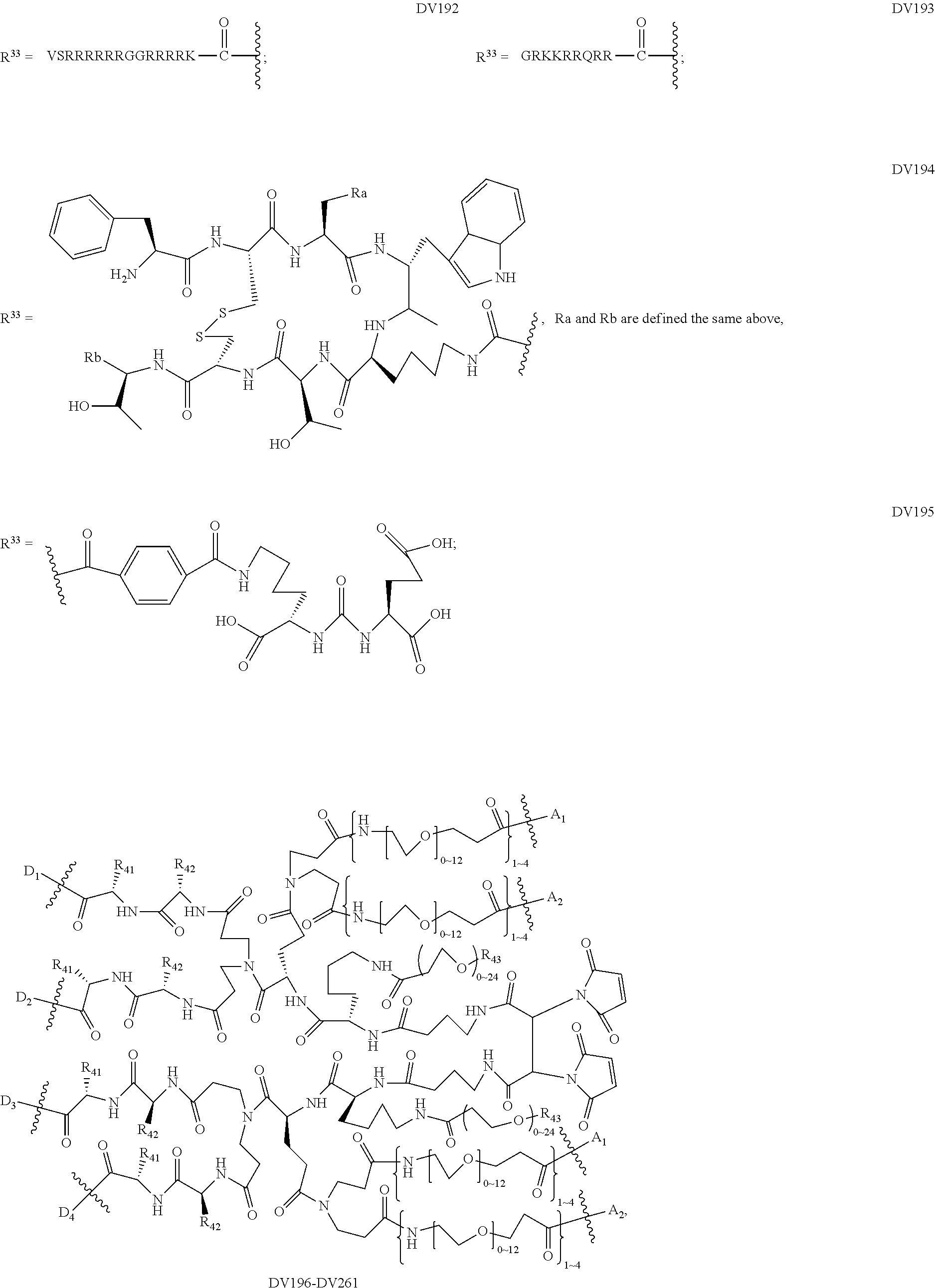
wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₁₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H4OH; R₄₃ is H, CH₃, CH₂CH₃, C(O)H, and C(O)CH₃; D1, D₂, D3 and D4 are cytotoxic drug and A and A₂ are small molecular ligands or drugs; 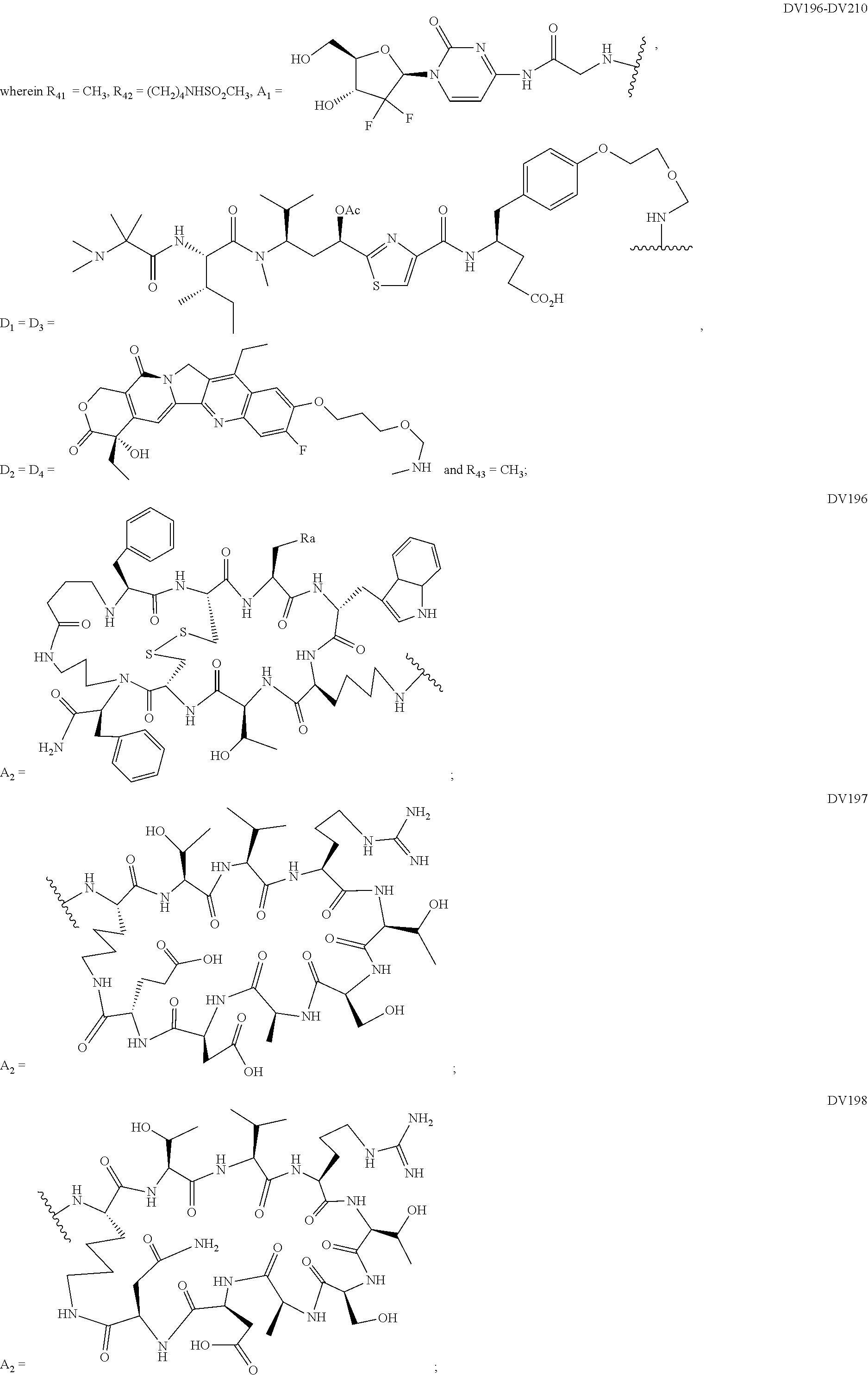 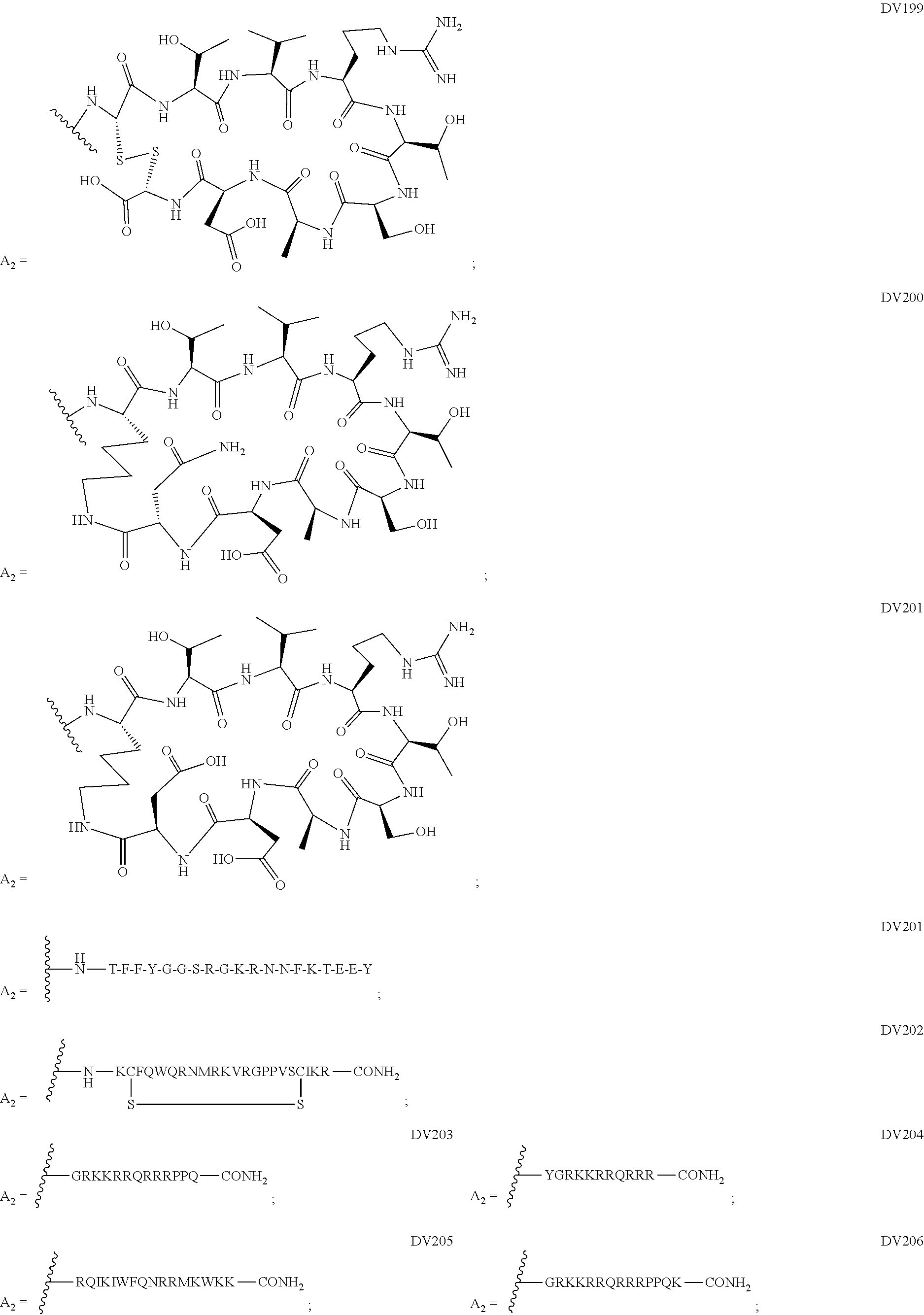 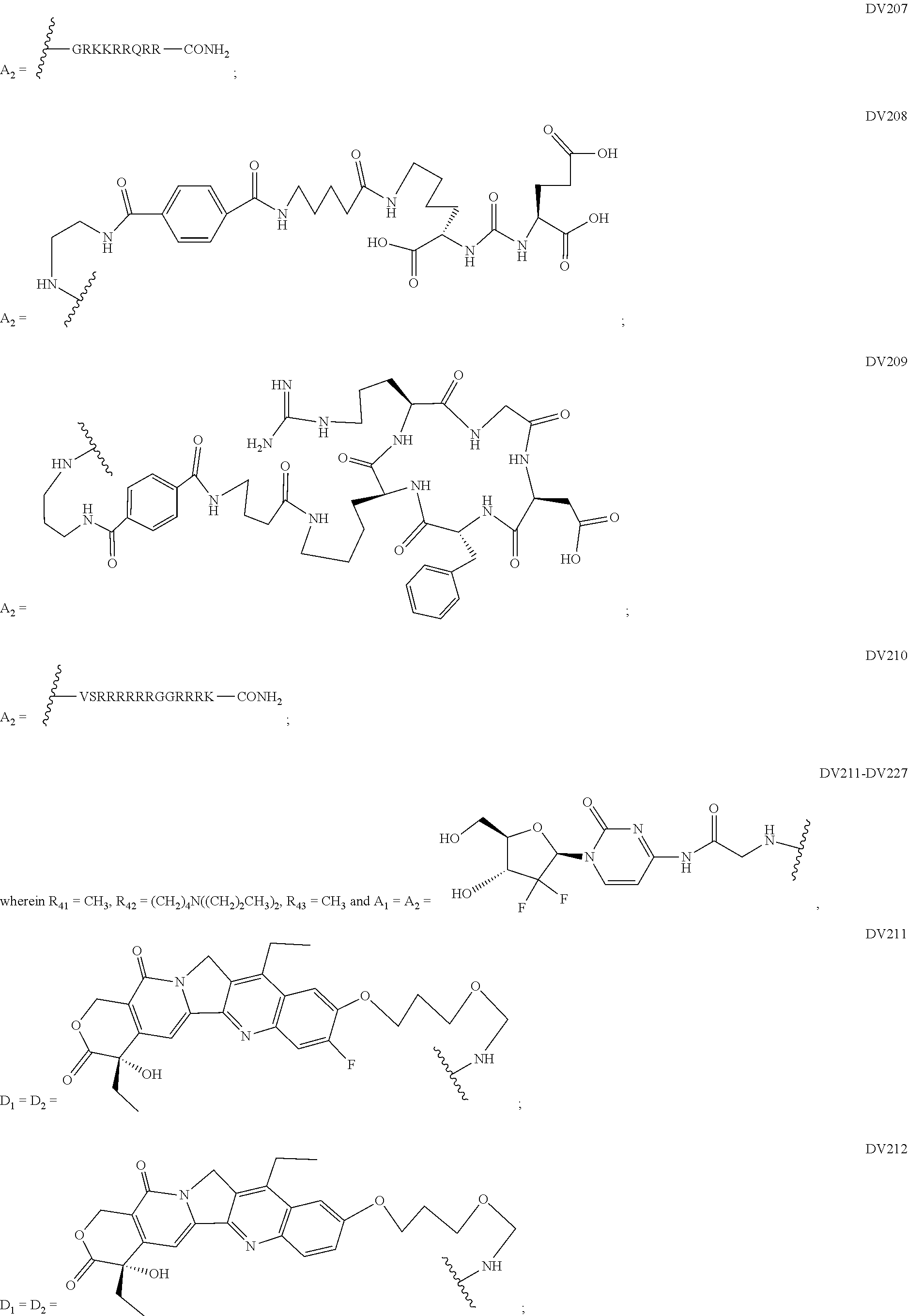 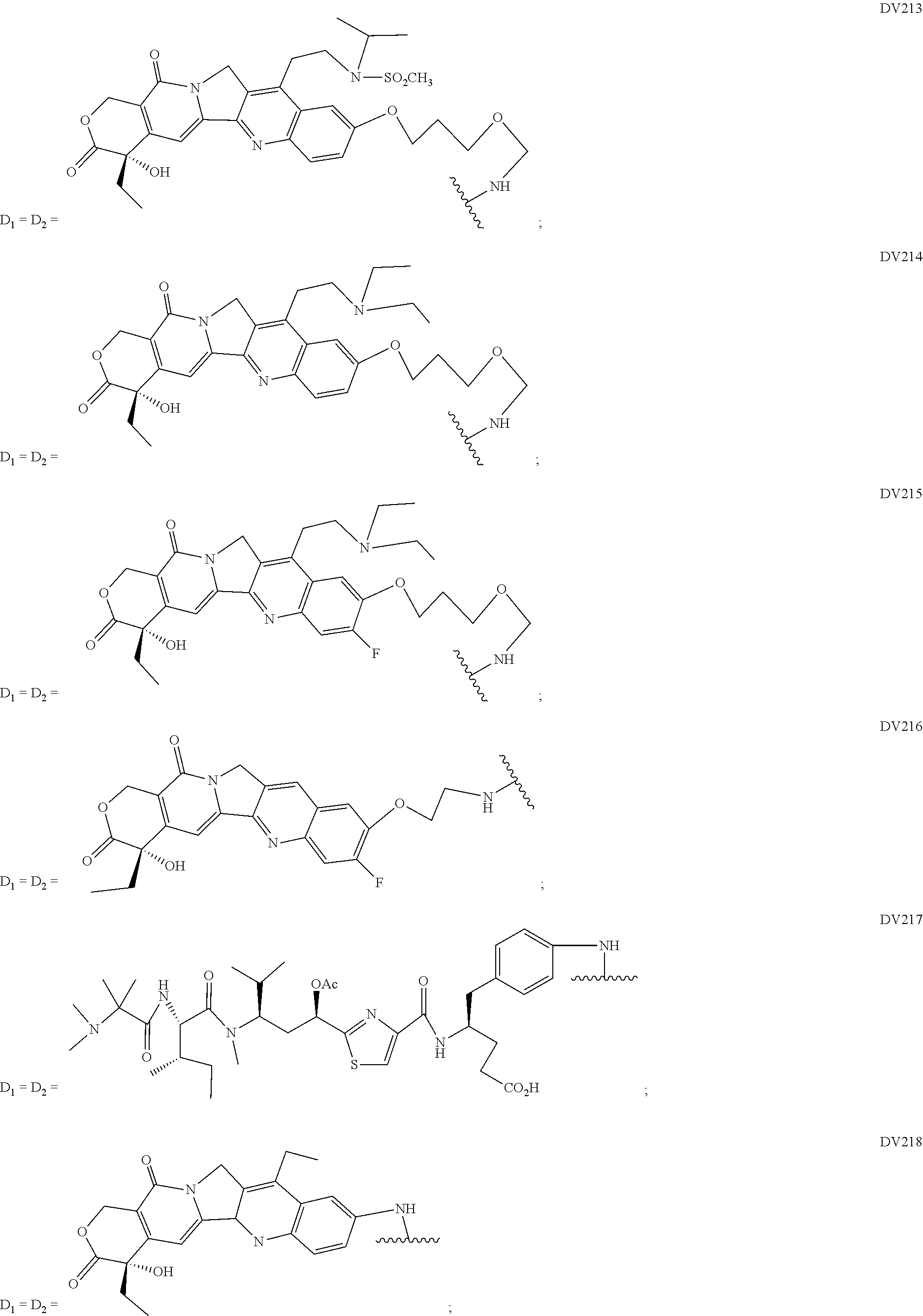 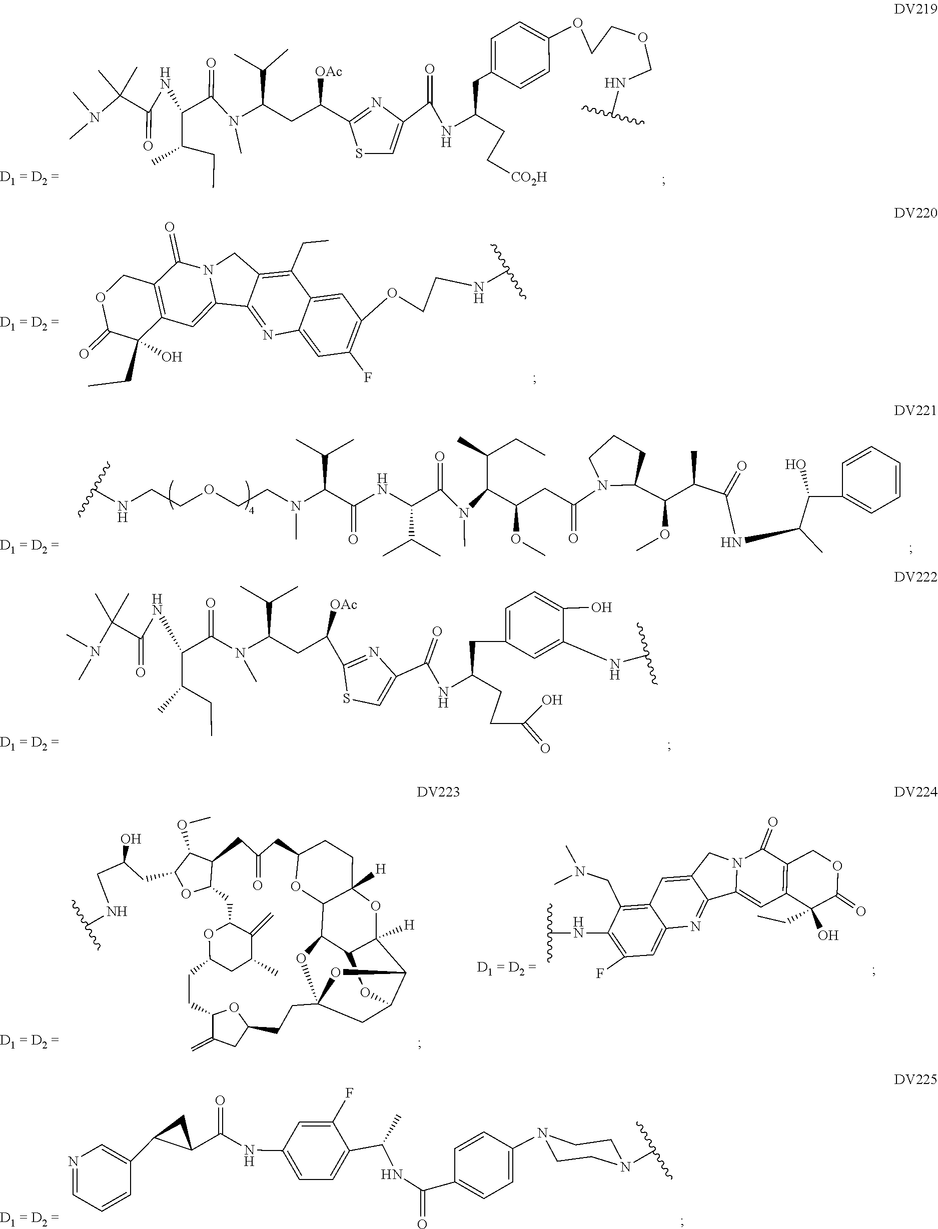 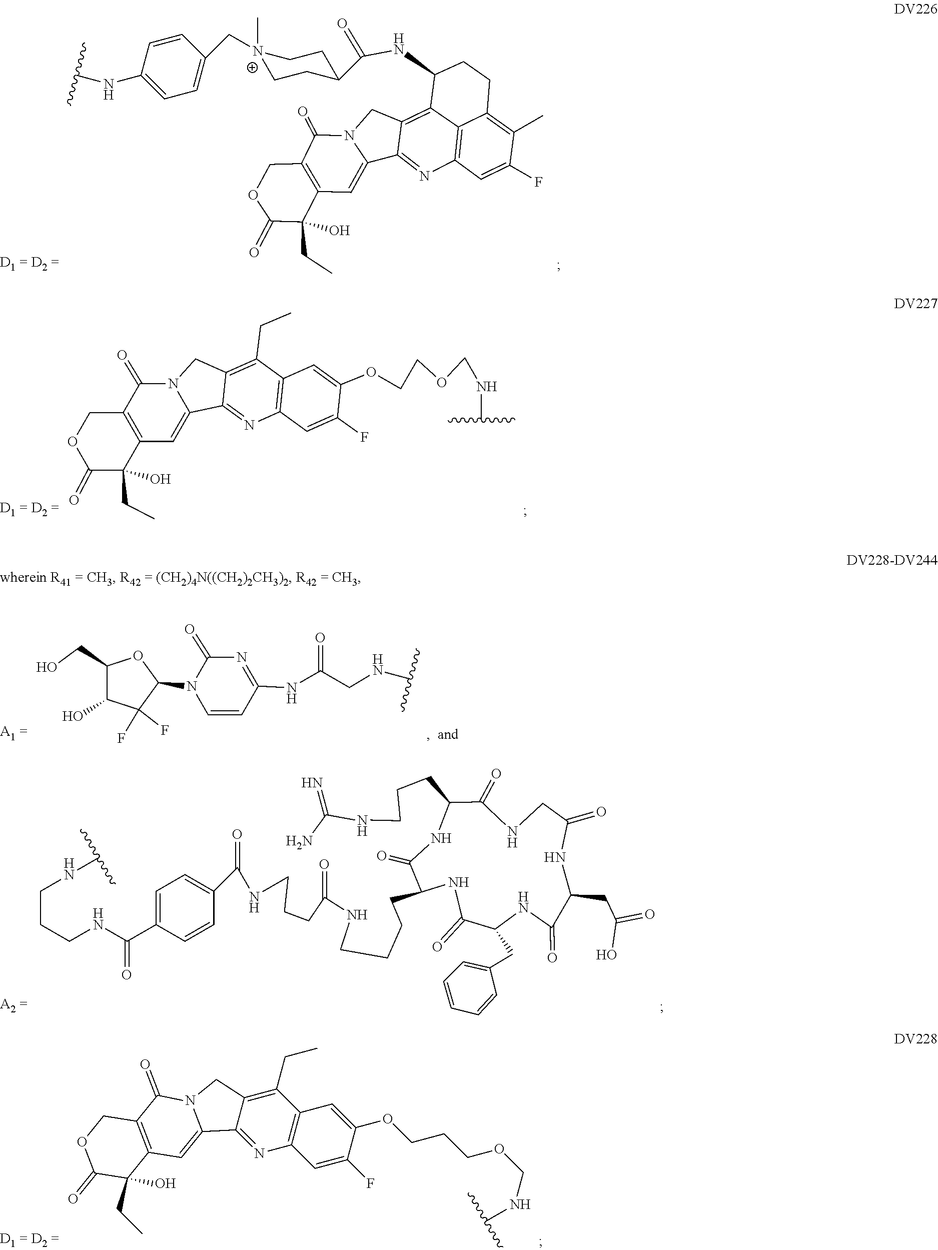 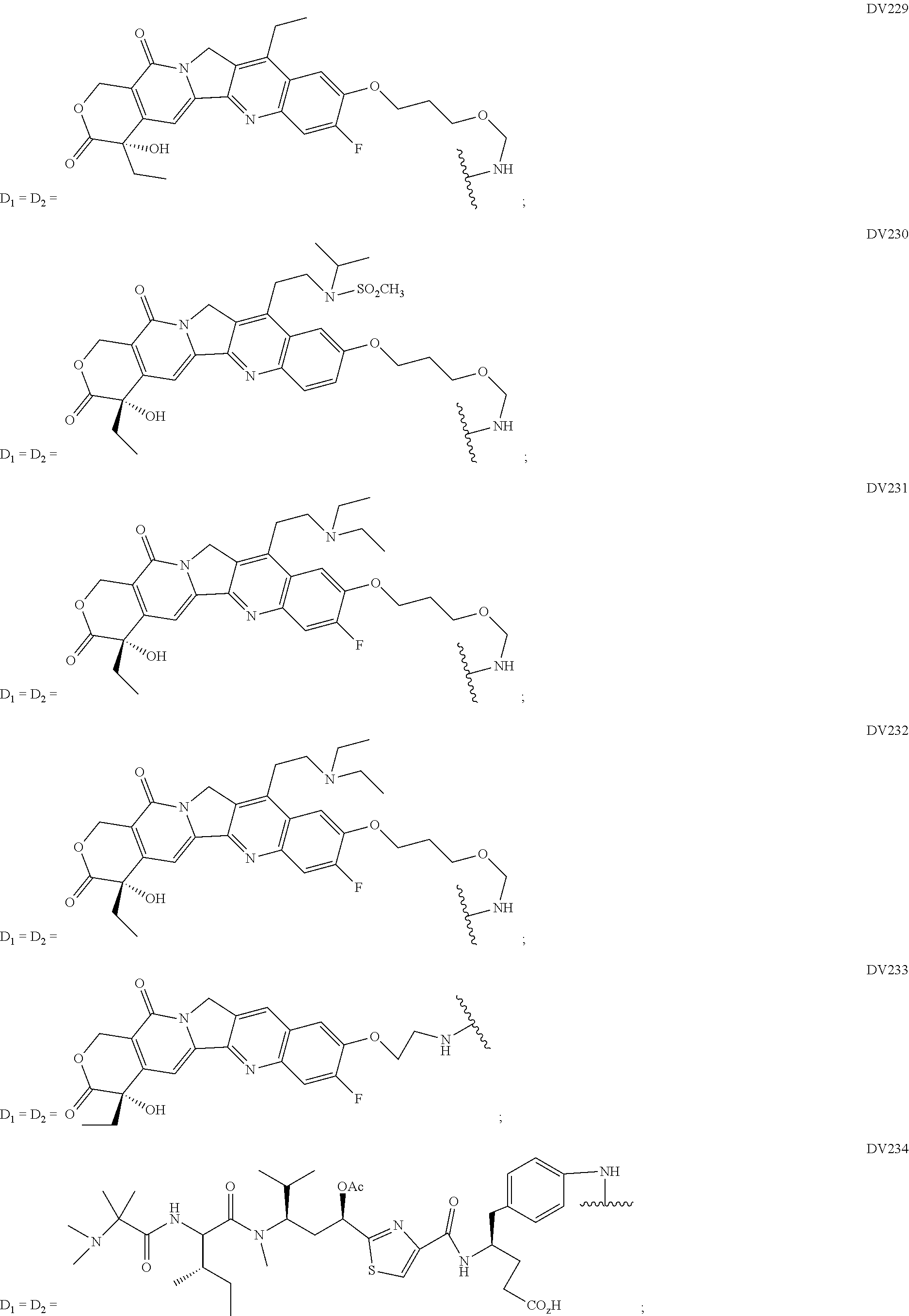 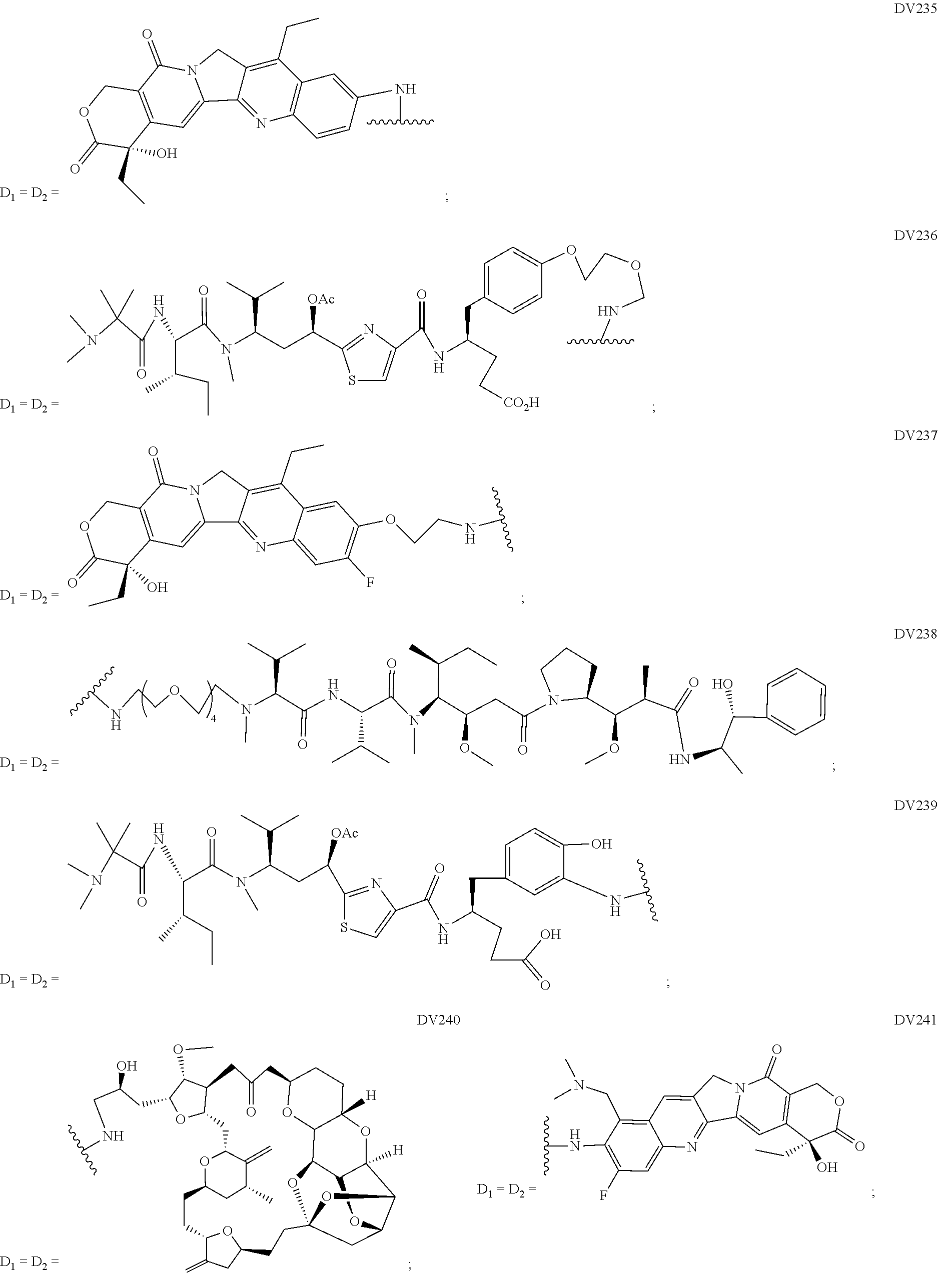 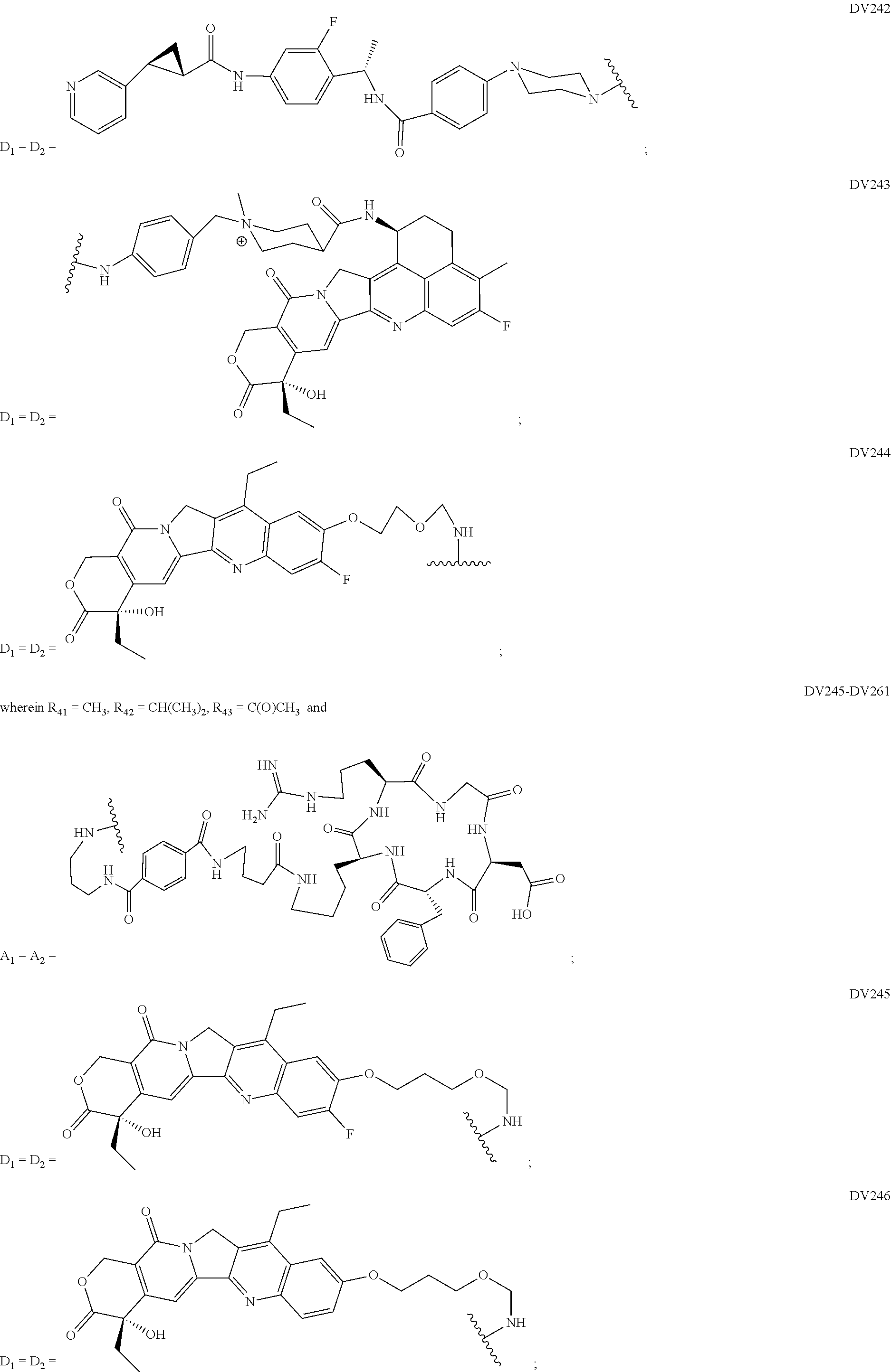 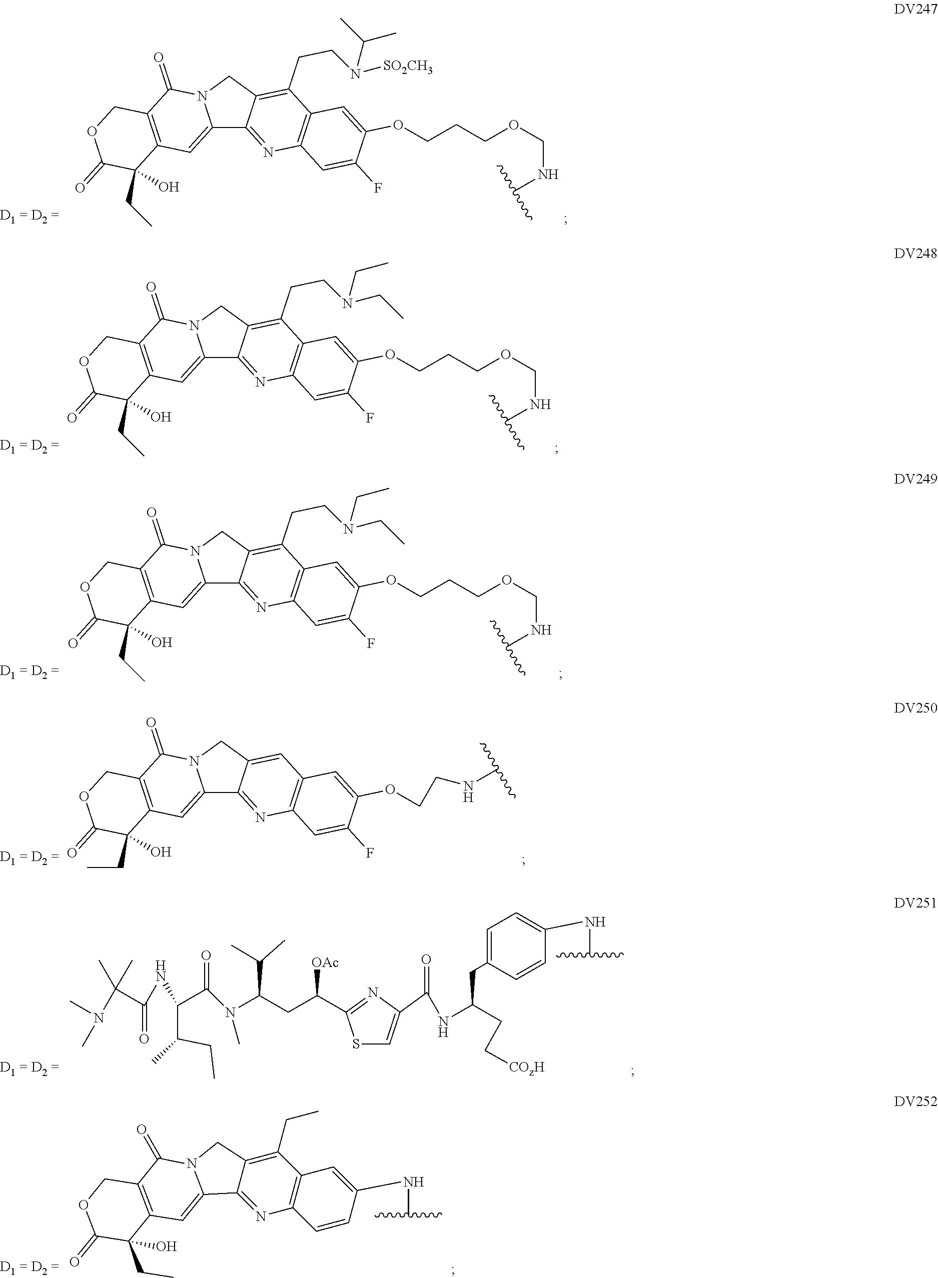 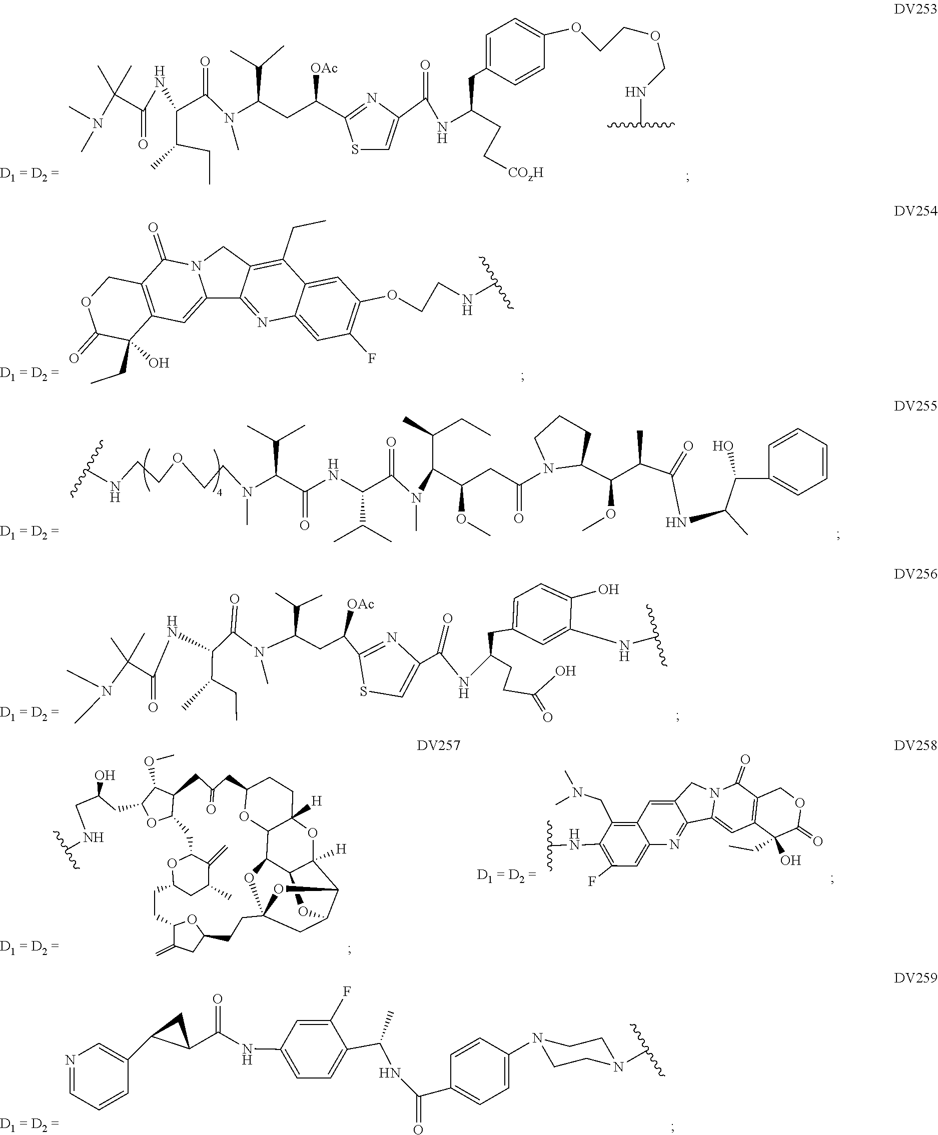 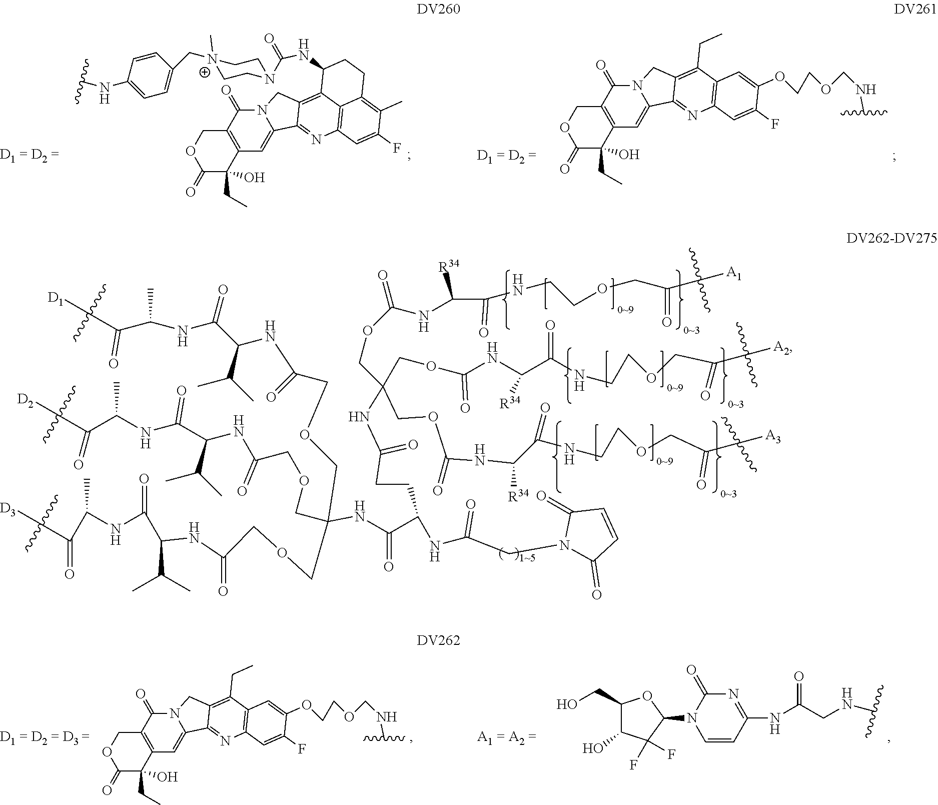 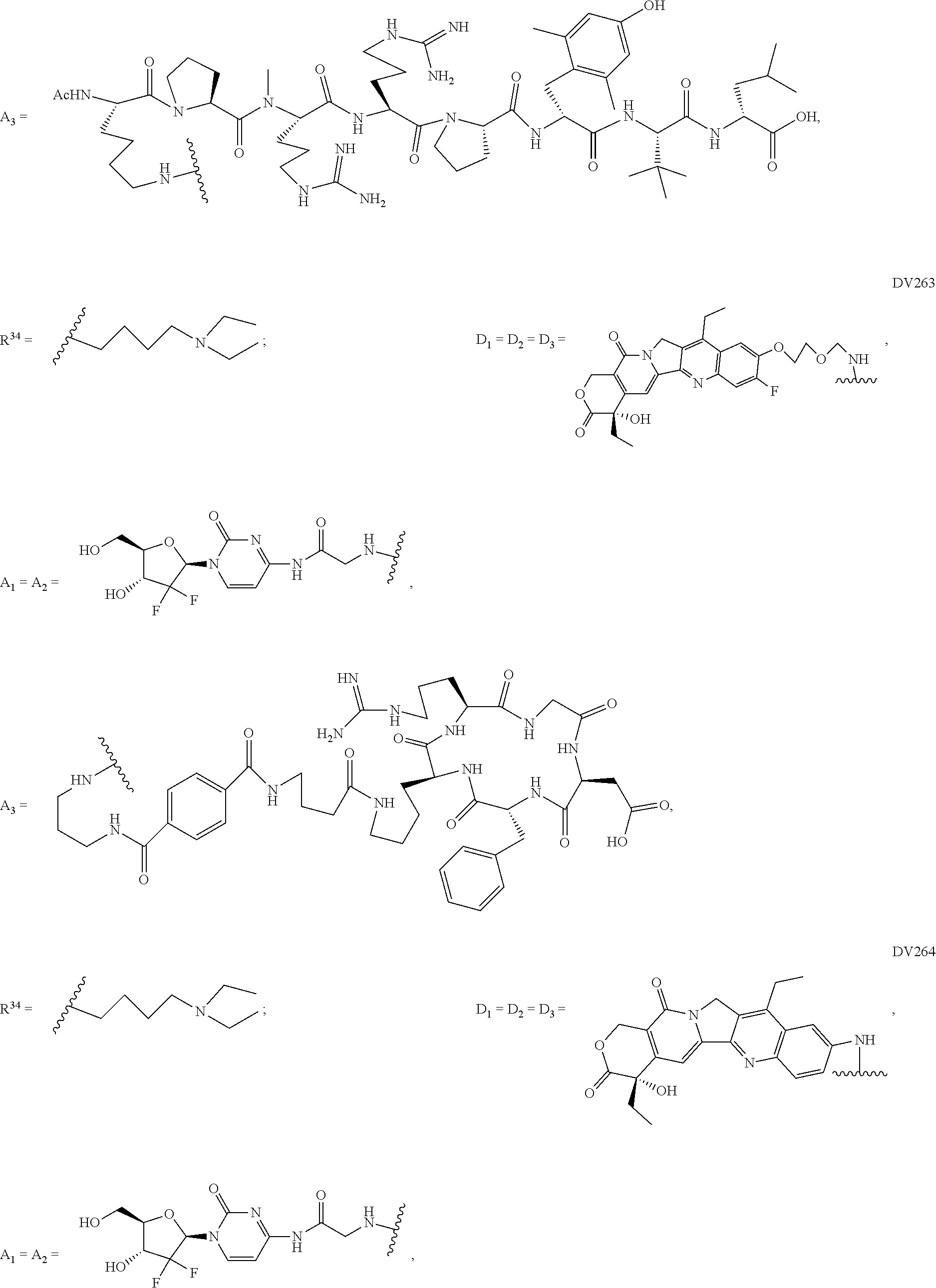 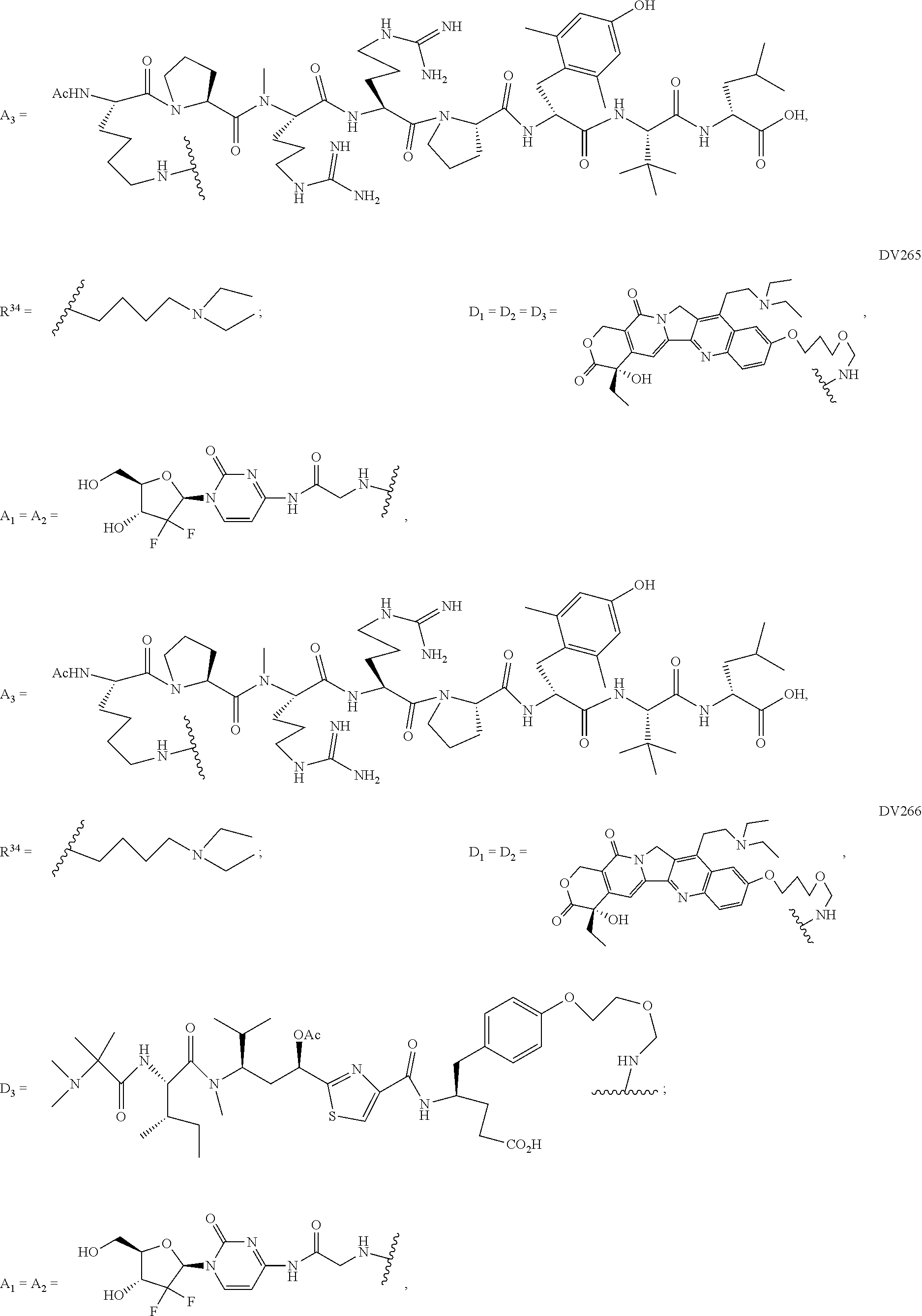 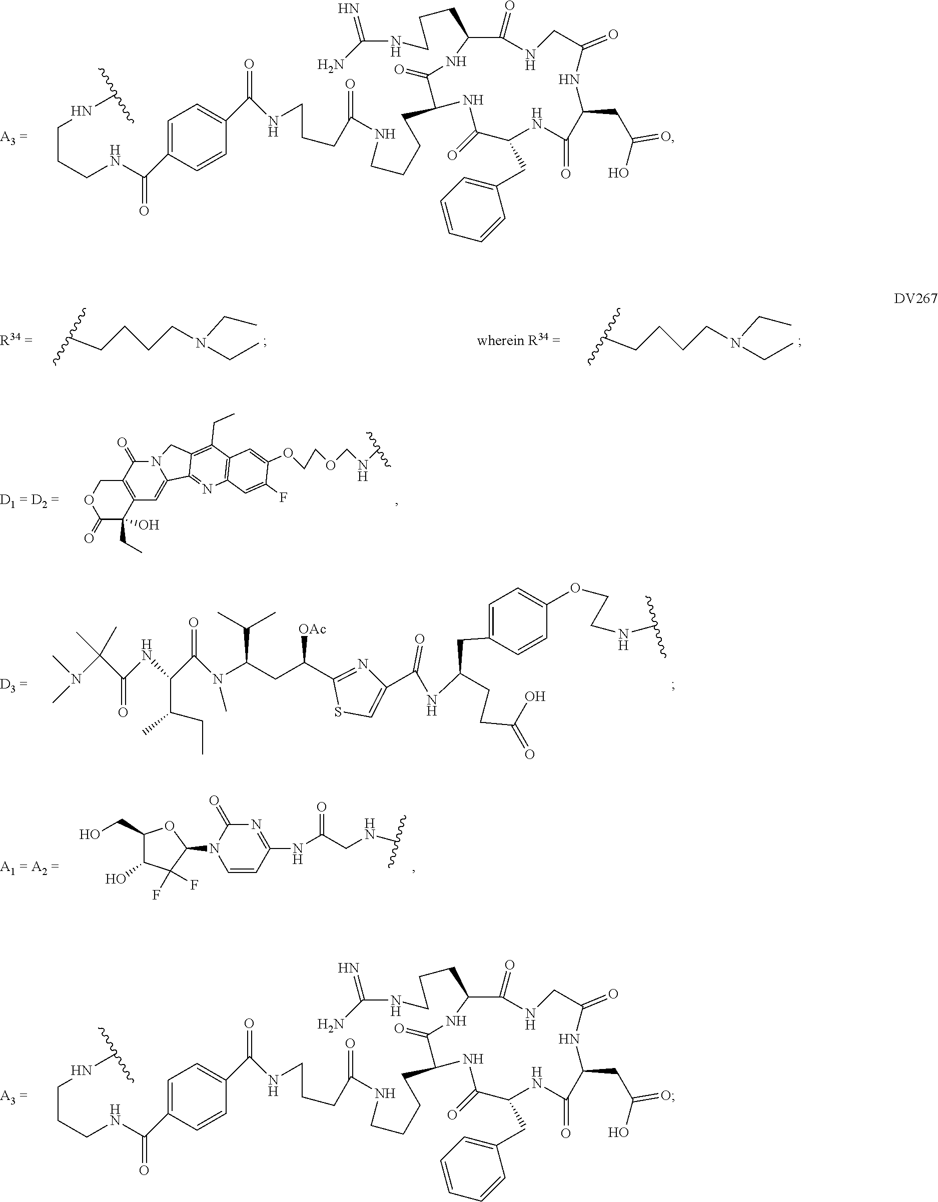 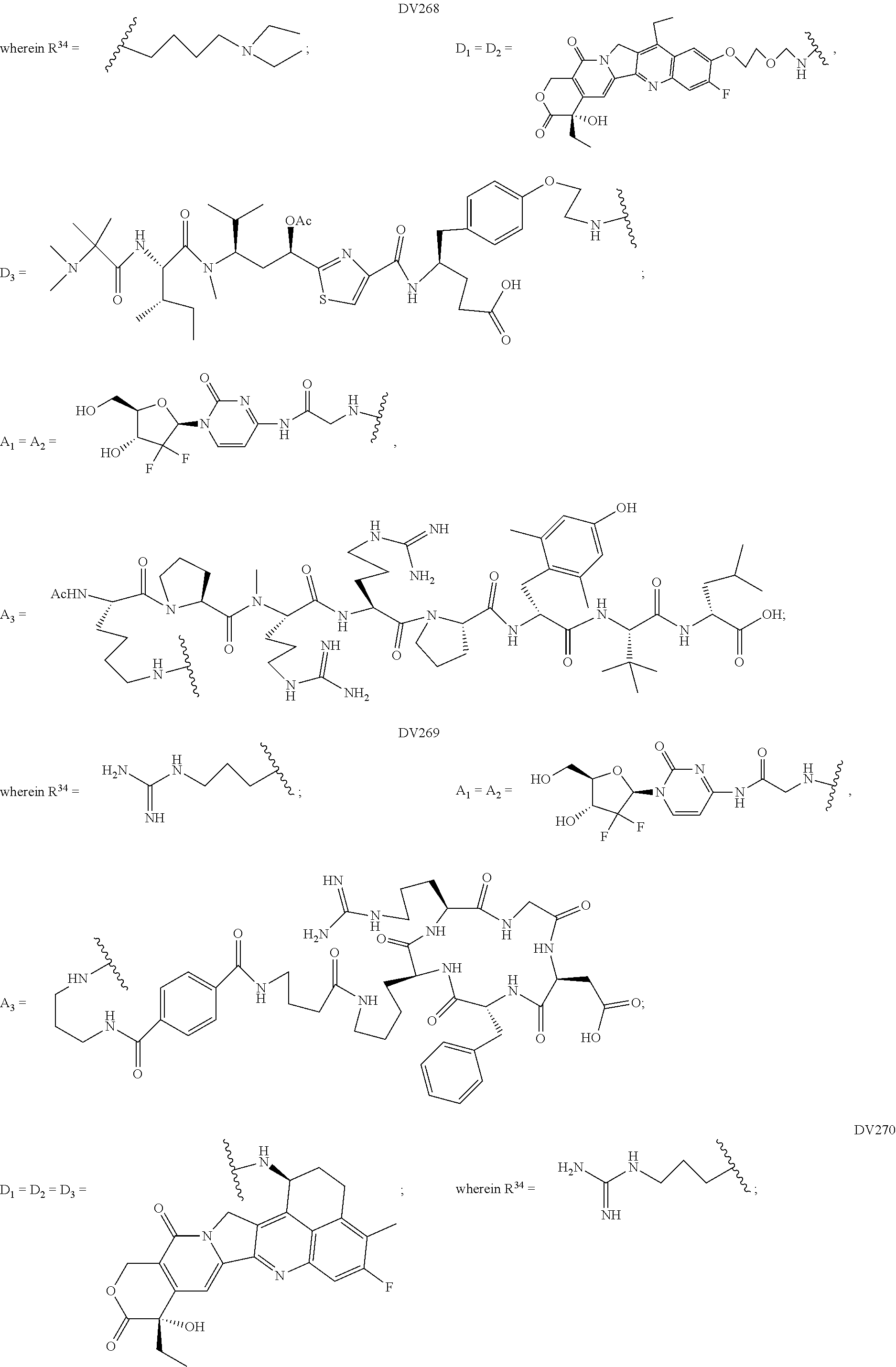 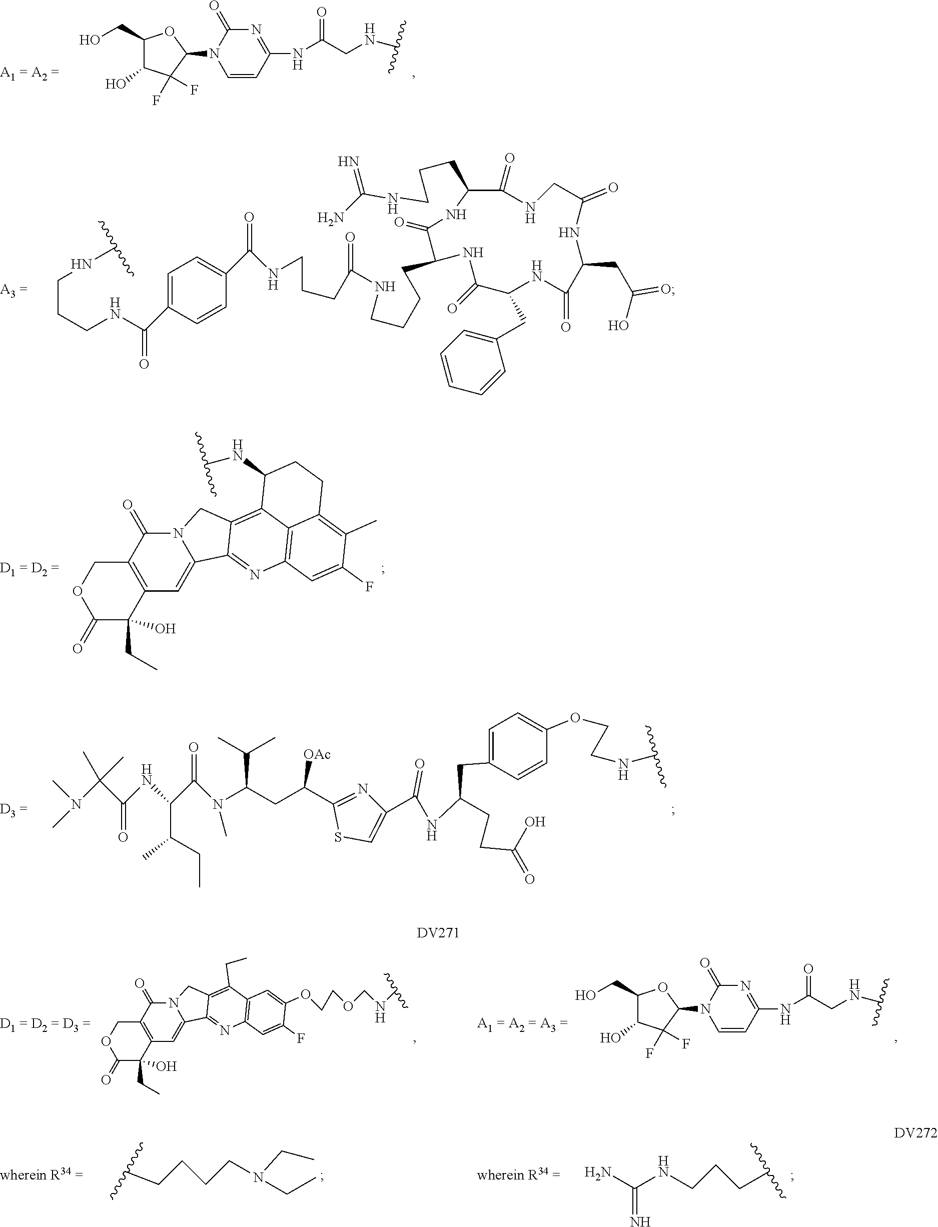 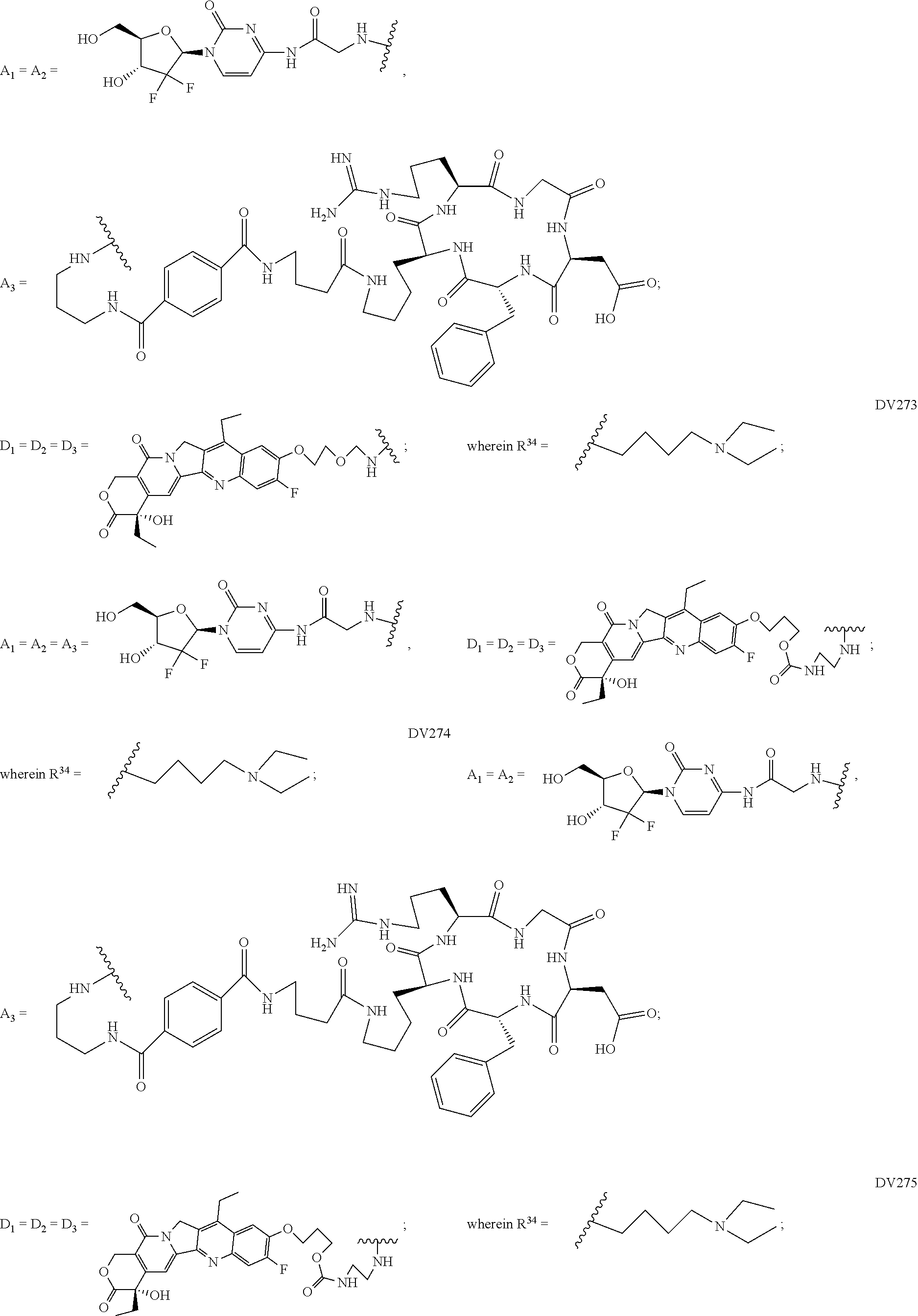 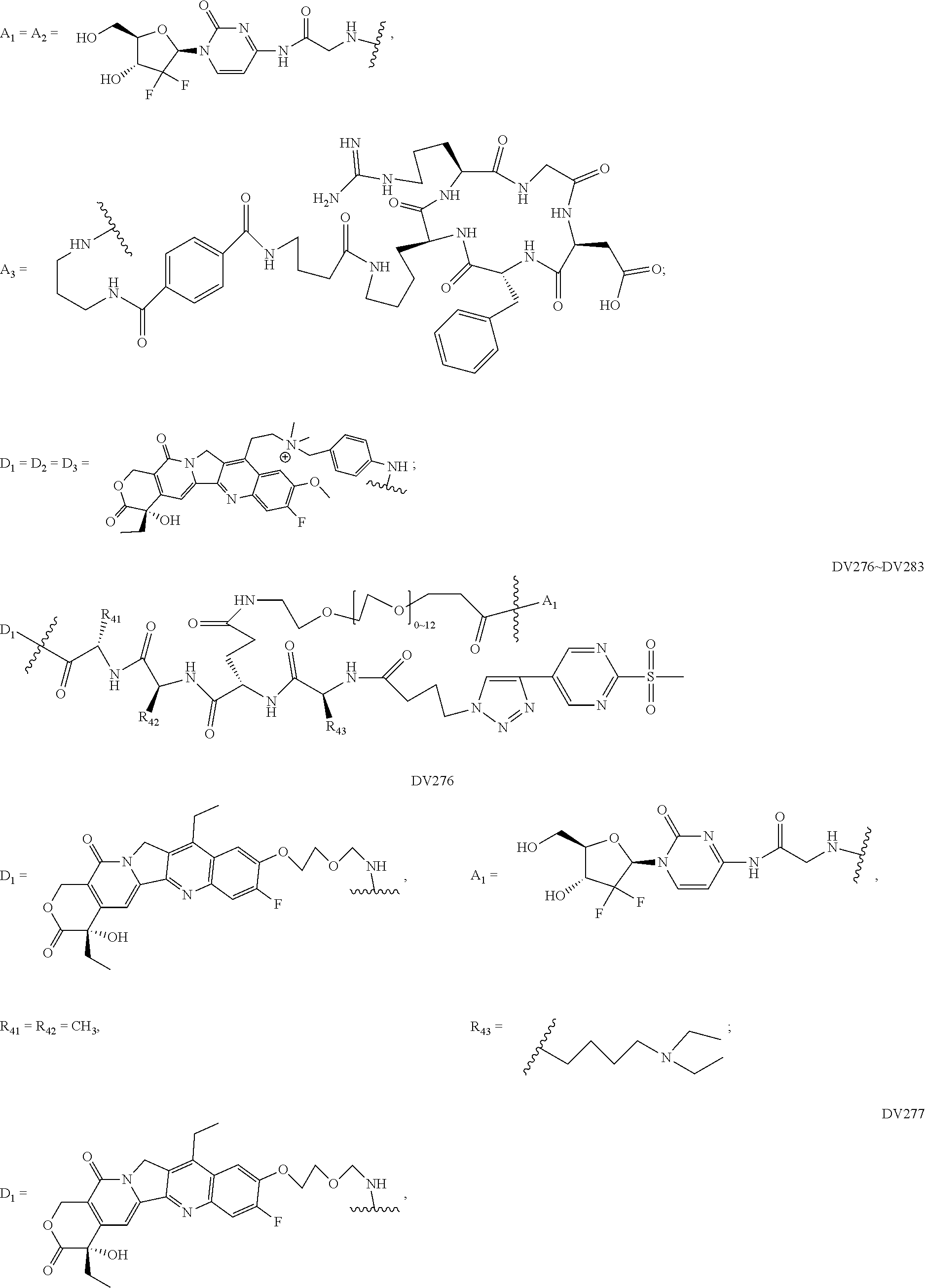 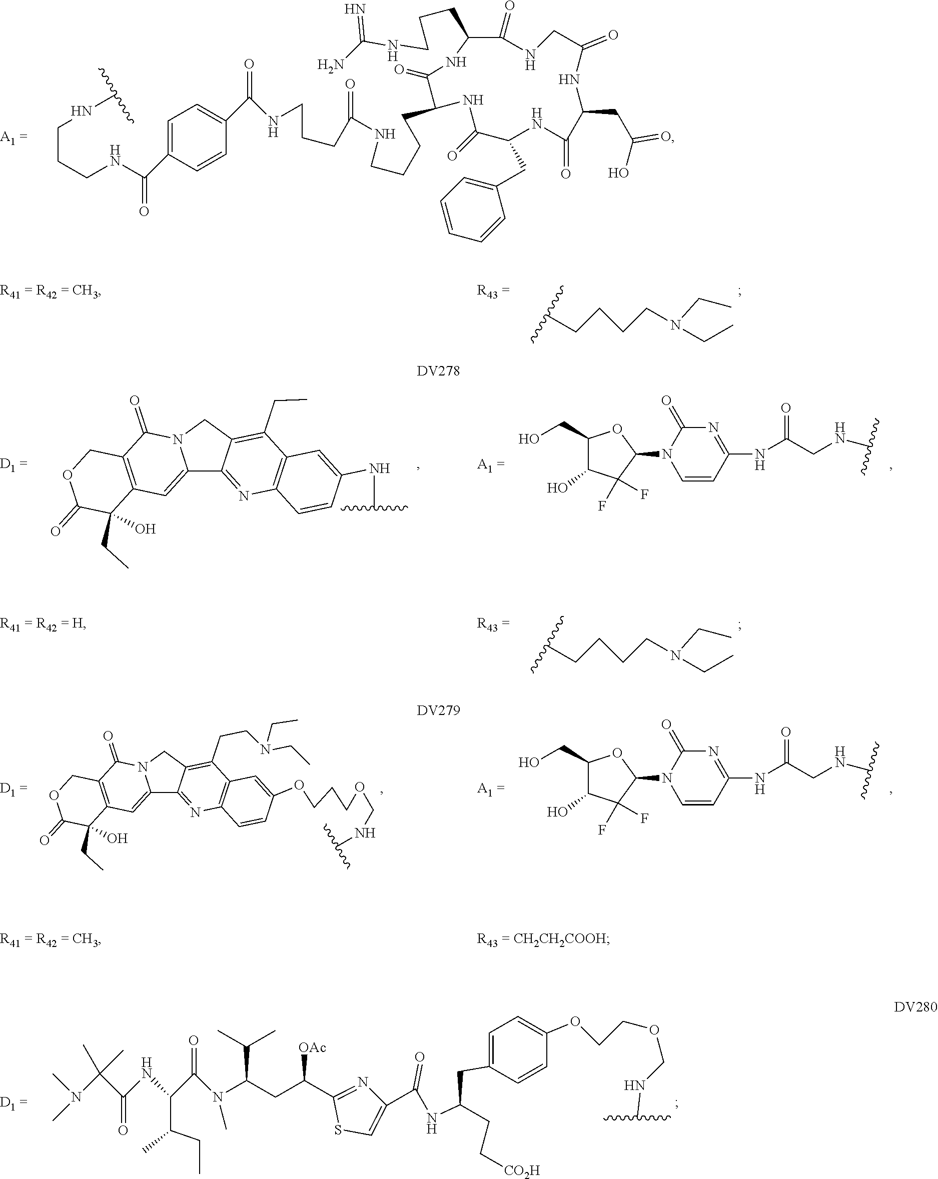 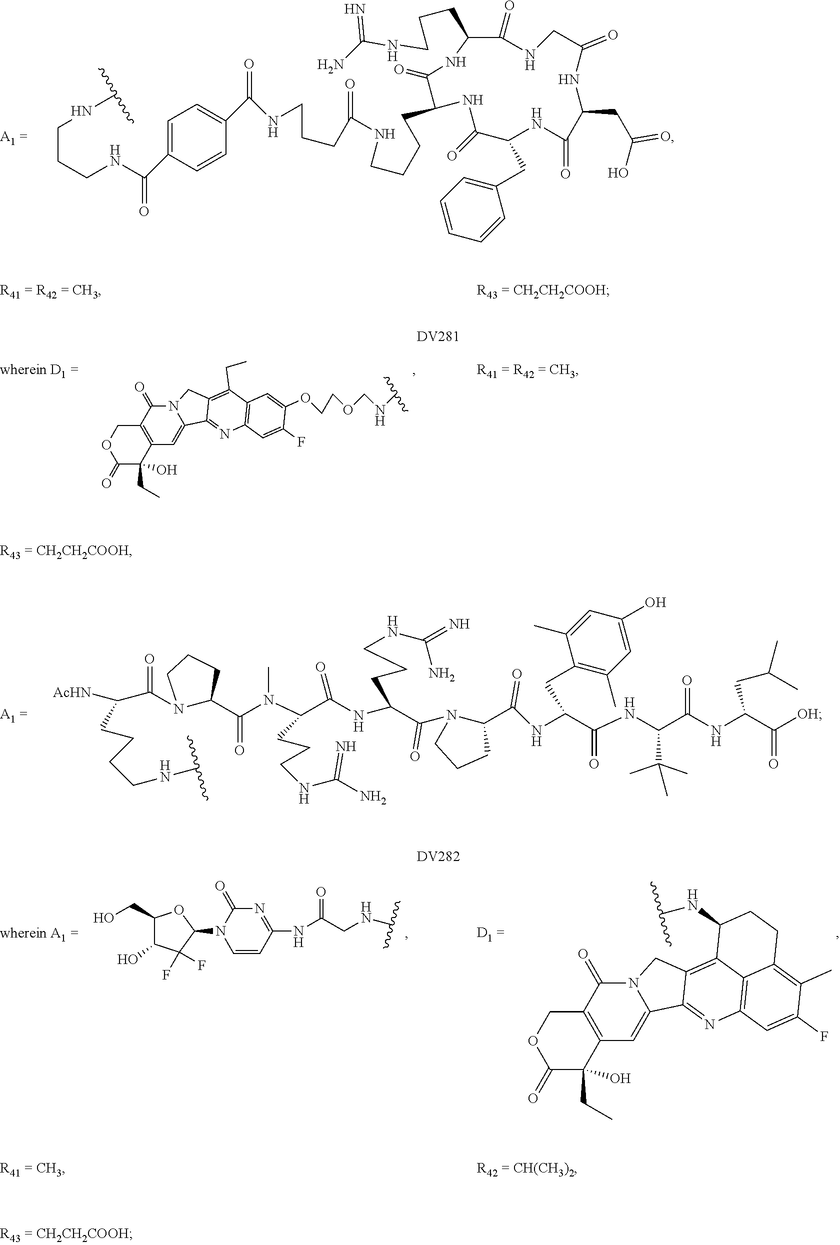 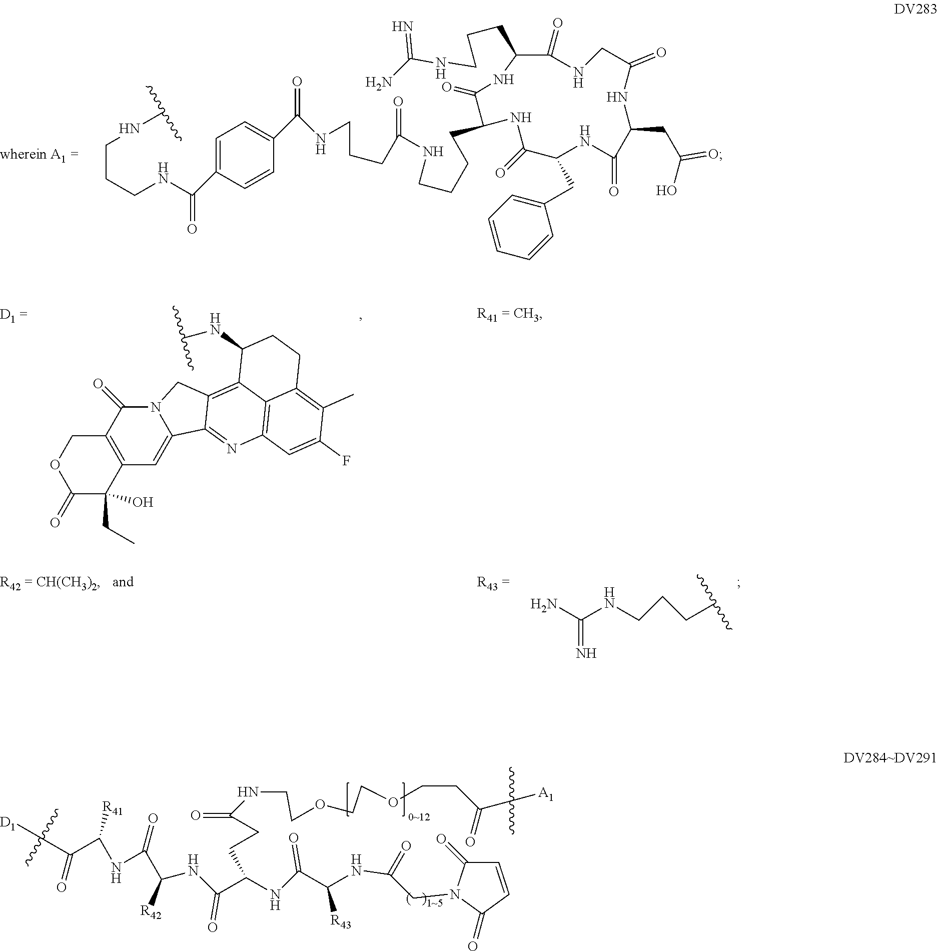 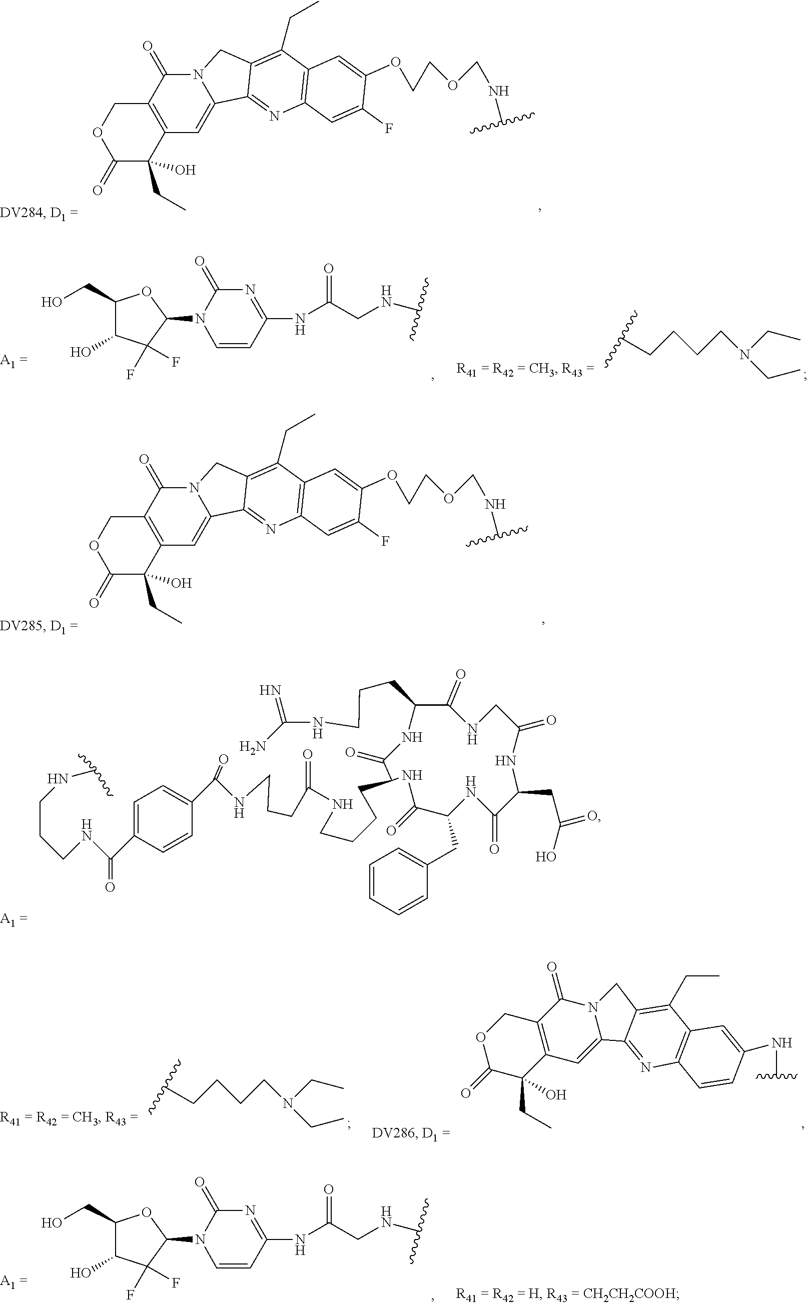 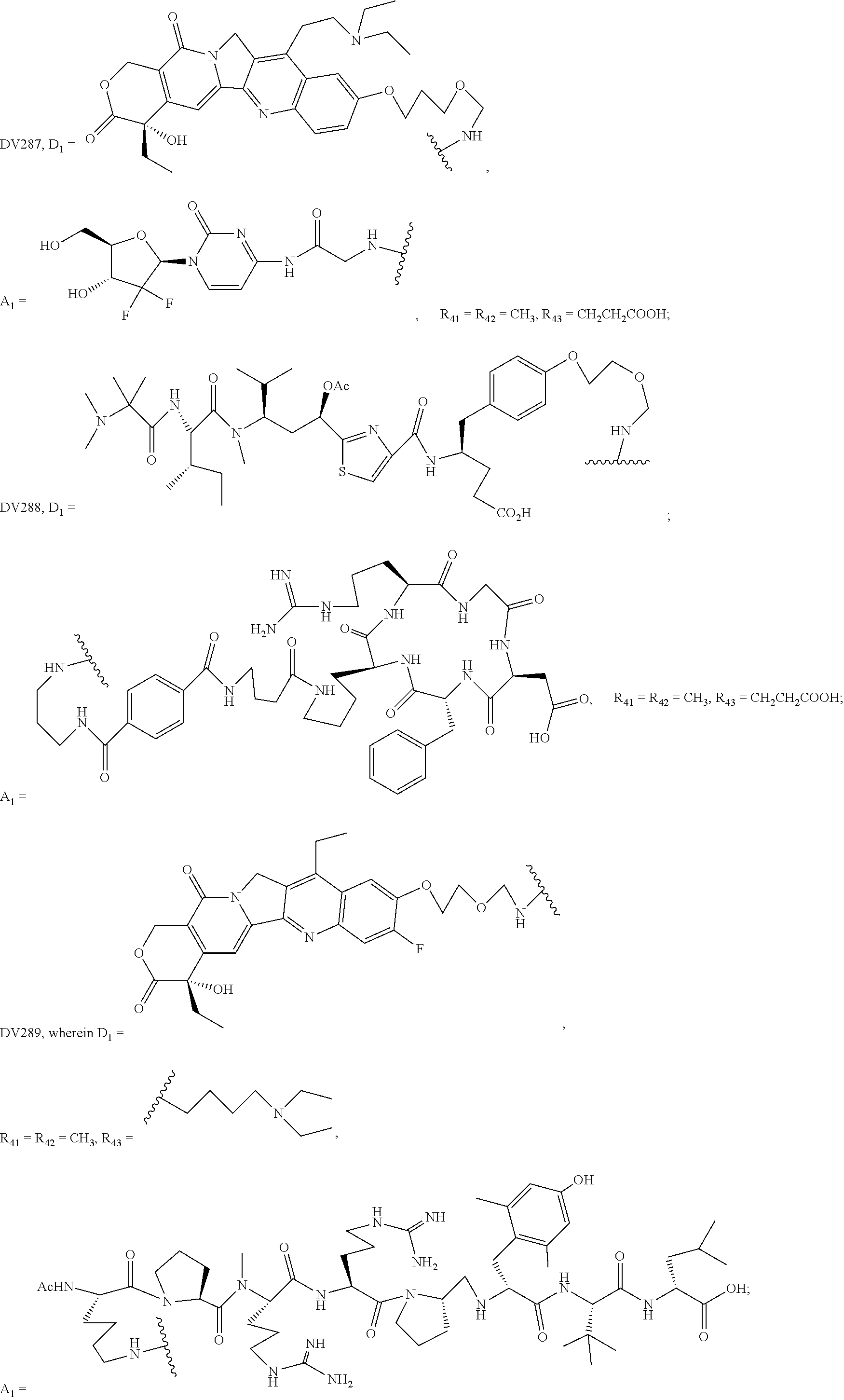 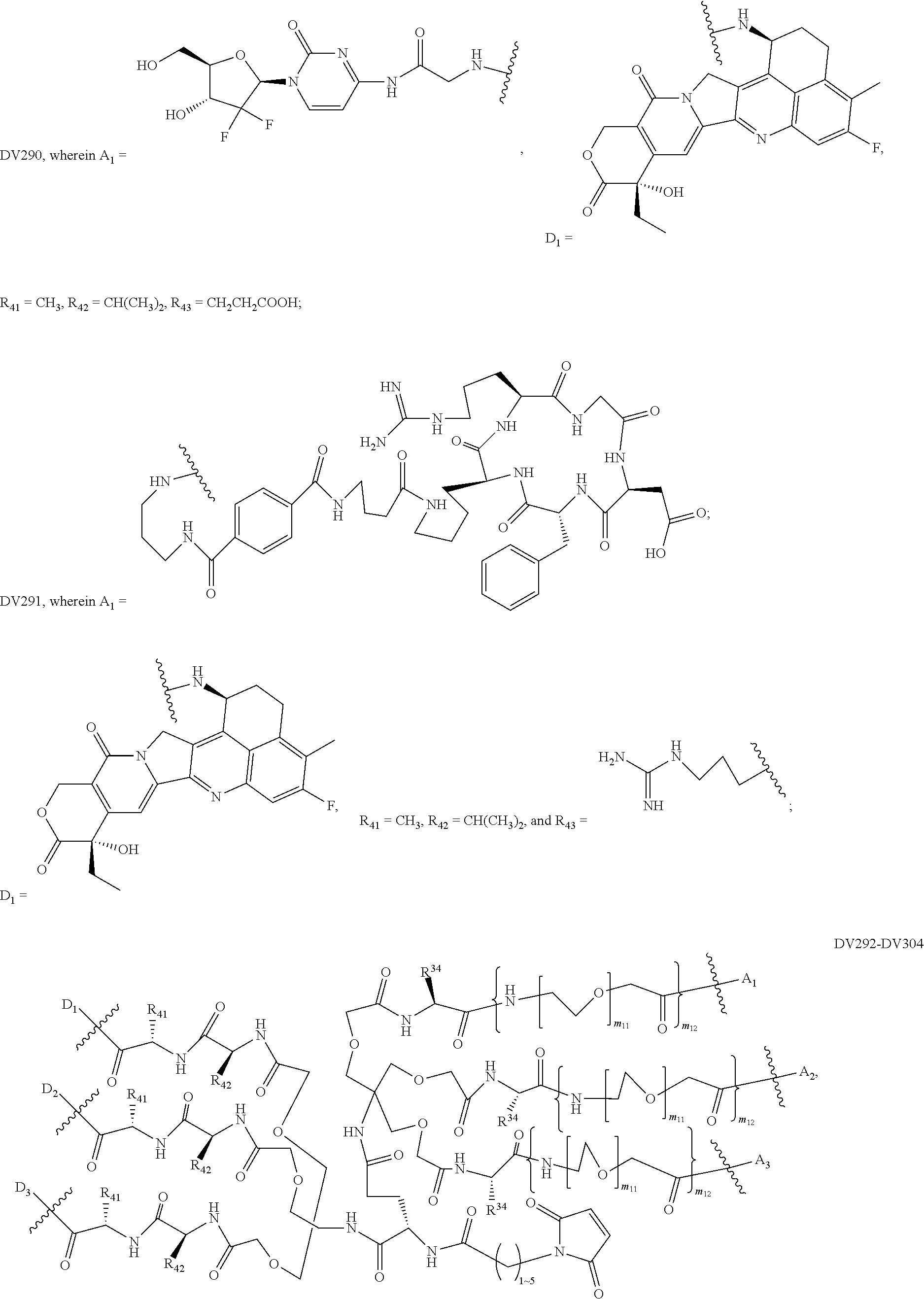
wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₁₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H4OH;
R³⁴ is H, CH₃, CH₂CH₃, C(O)H, and C(O)CH₃; D1, D₂, D3 and D4 are cytotoxic drug and A₁, A₂ and A₃ are small molecular ligands or drugs m₁₁ is a number of 0, 1, 2, 3, 4, 5, 6, 7, 8, 9 and m₁2 is a number of 0, 1, 2, 3 and 4; when number is 0 for the brackets, it means the structure in the brackets can be omitted (crossed out), 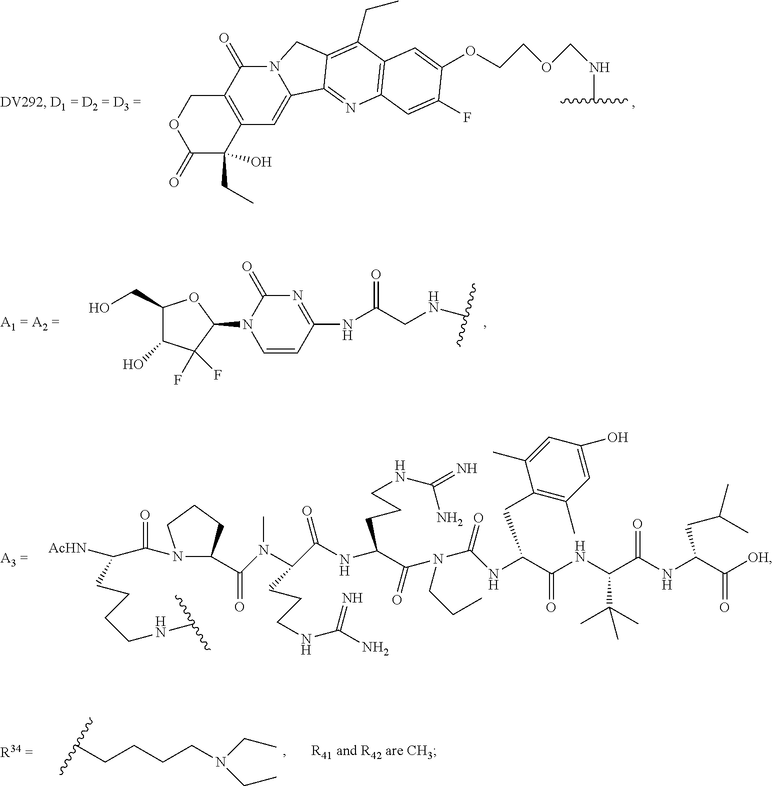 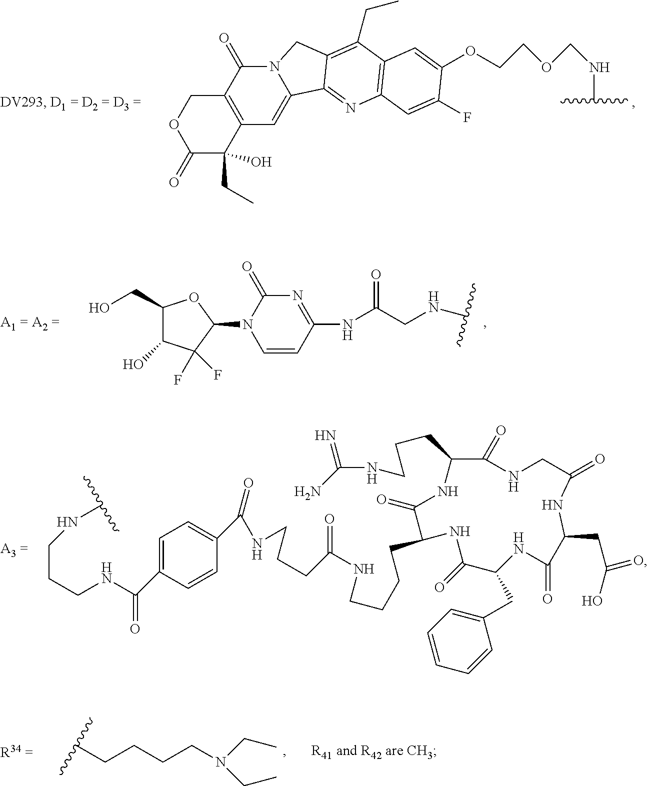 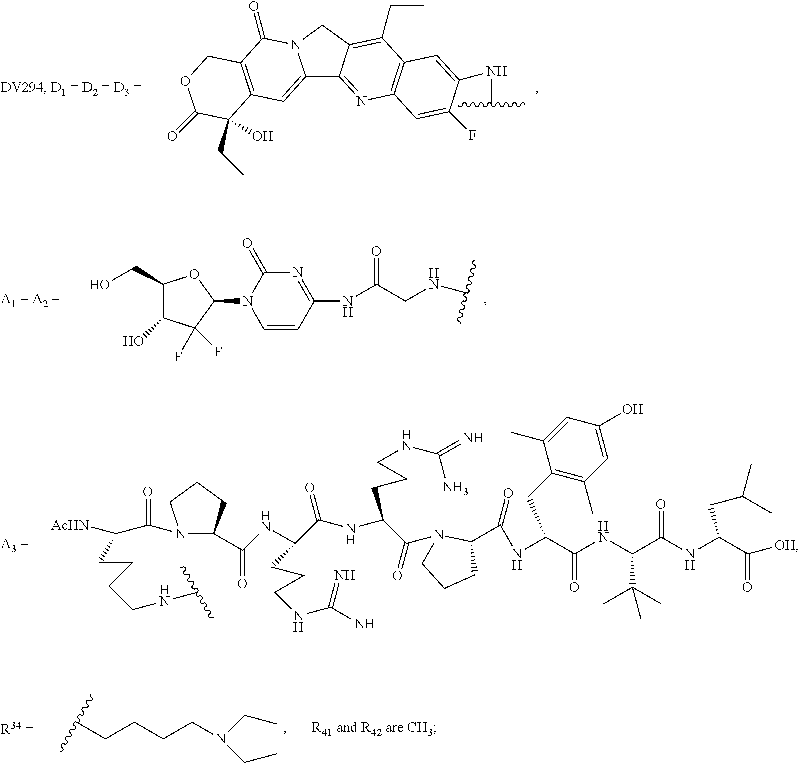 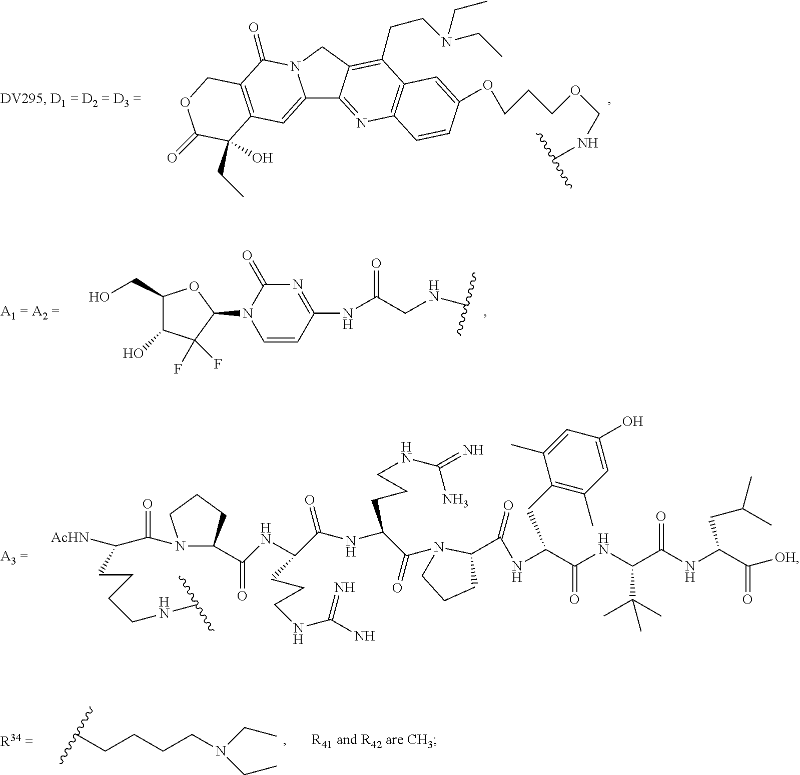 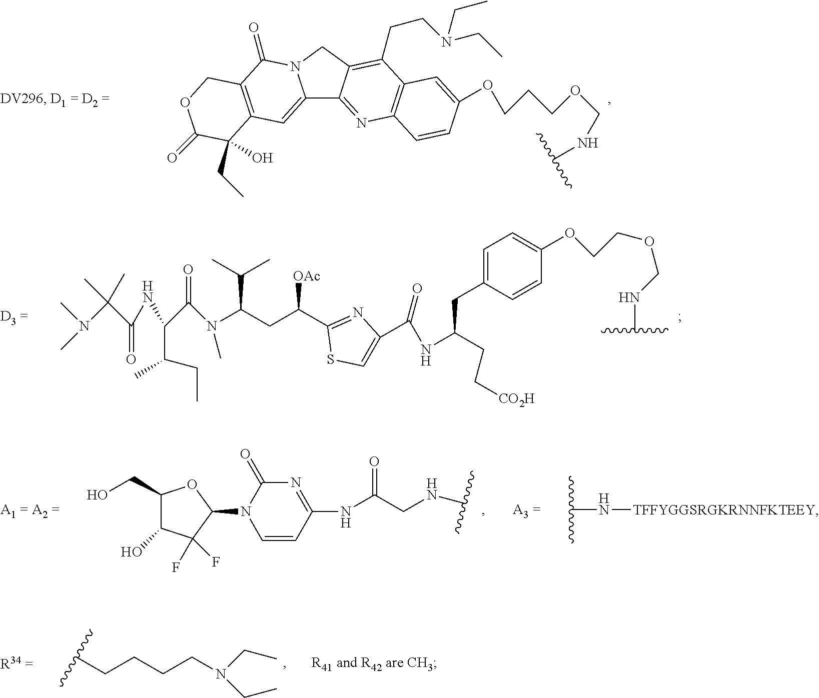 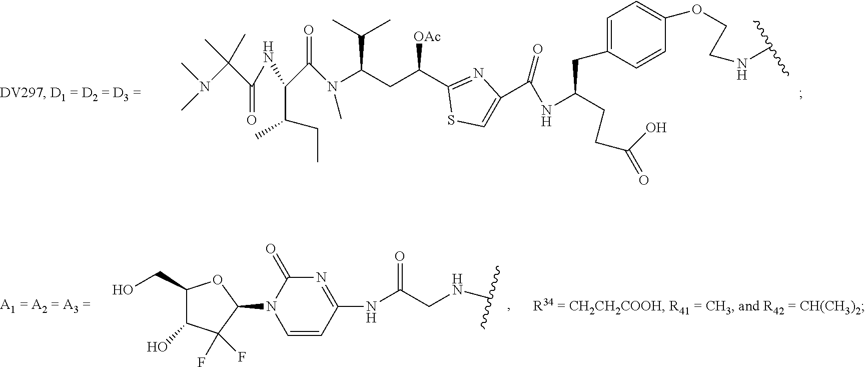 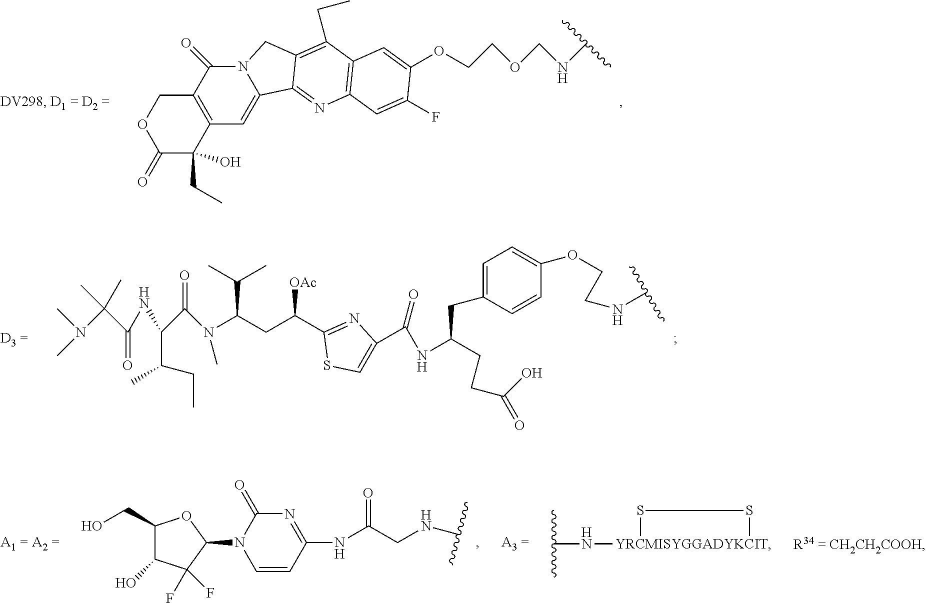 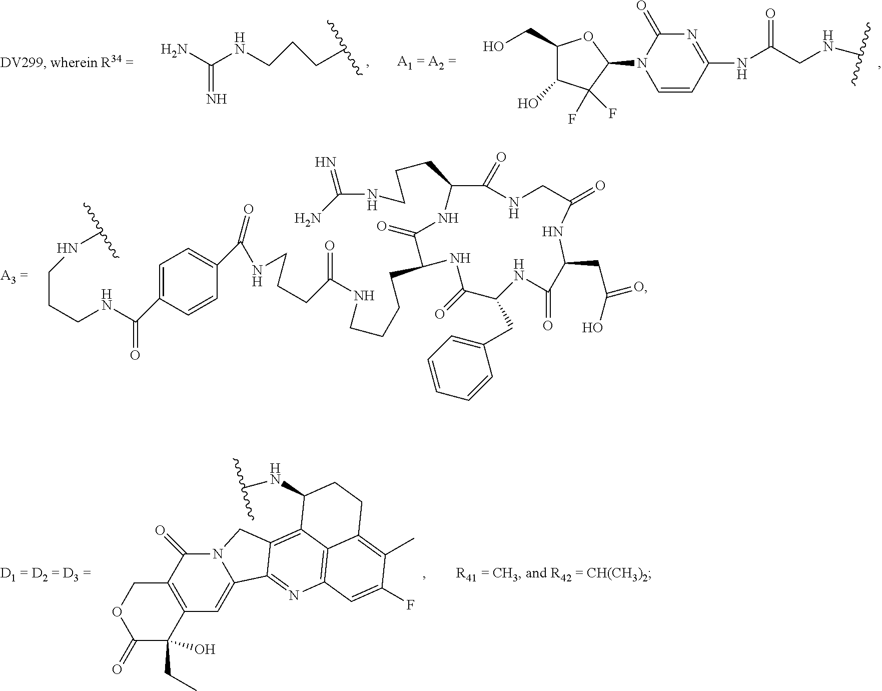 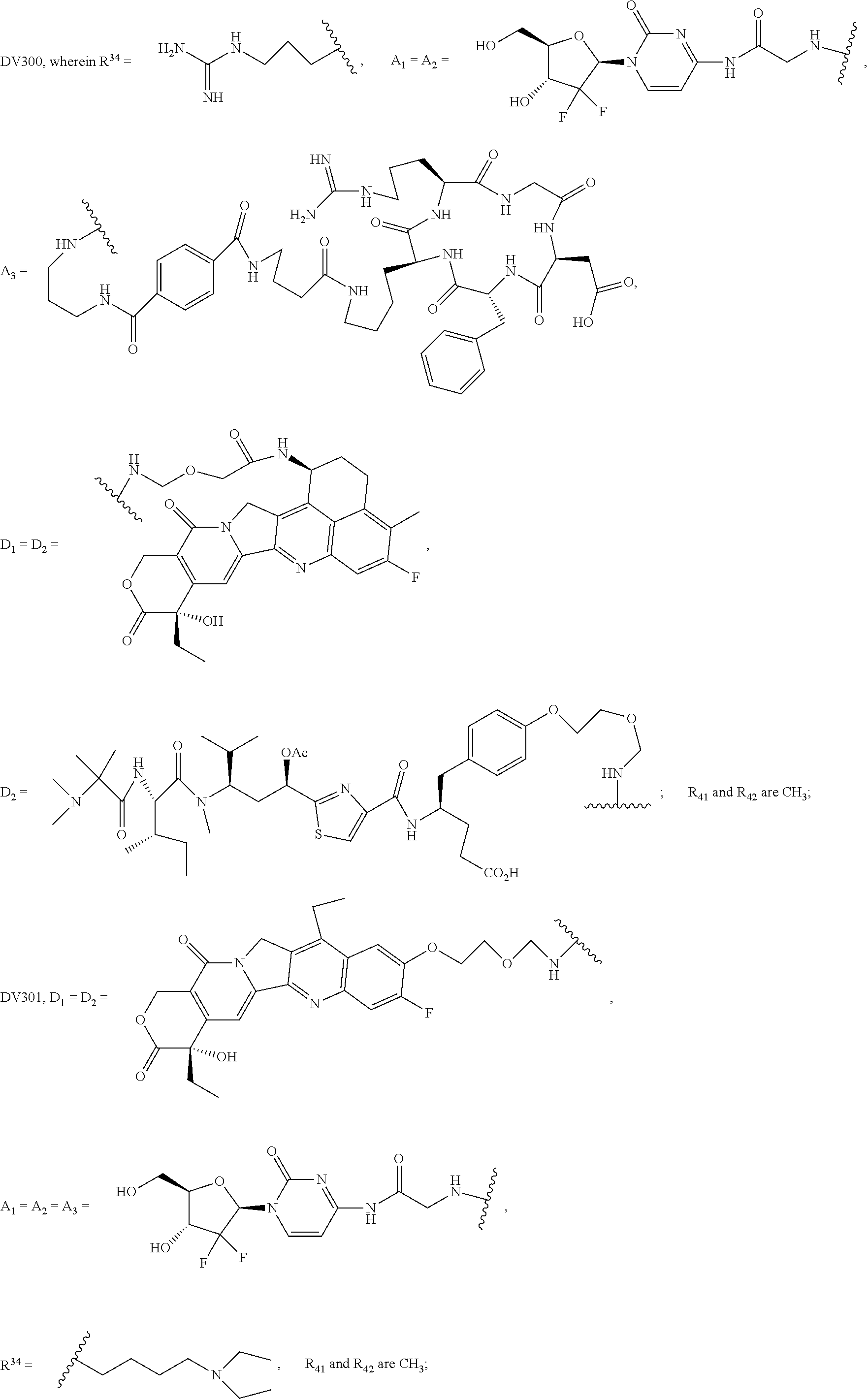 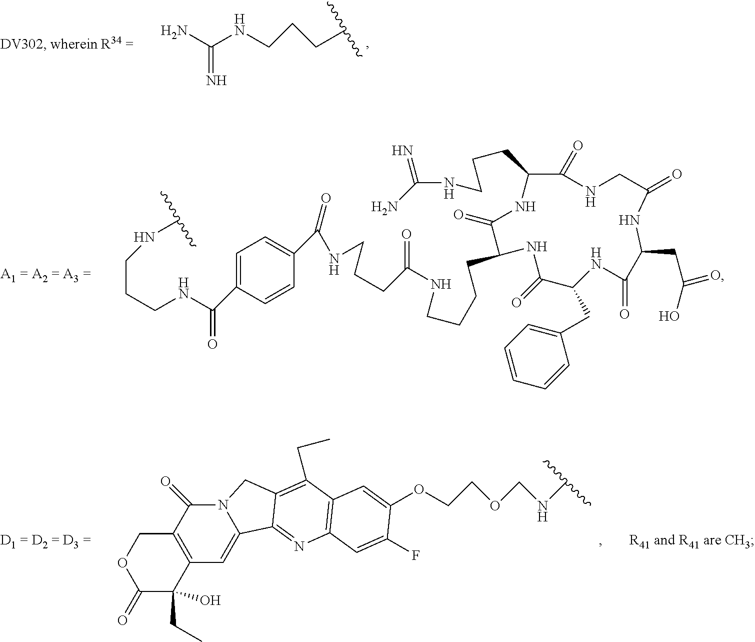 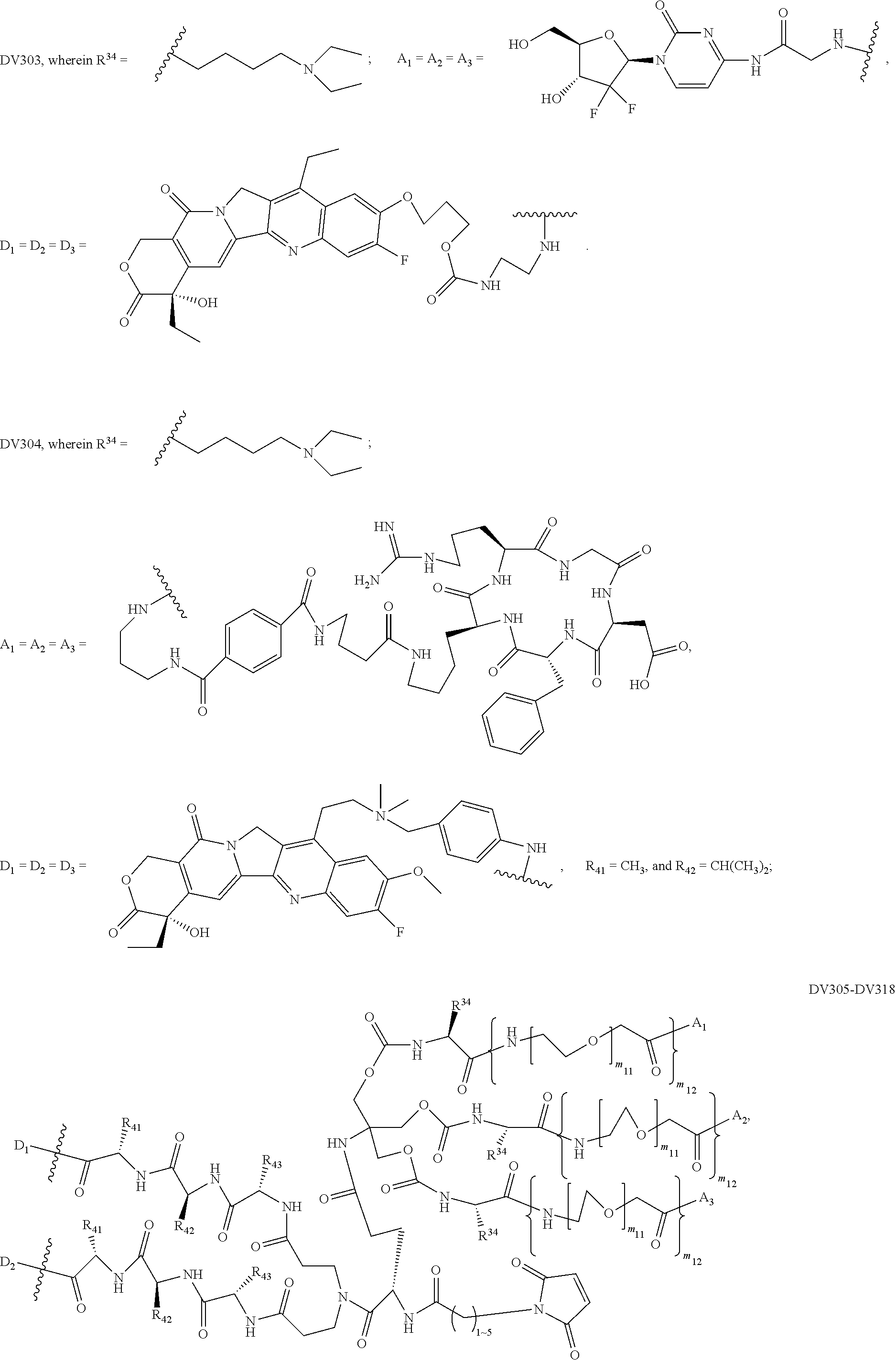
wherein R₄₁ and R₄₂ are independently H, CH₃, CH(CH₃)₂, CH(CH₃)(C₂H₅), CH₂COOH, CH₂CH₂COOH, CH₂CONH₂, CH₂CH₂CONH₂, (CH₂)₄NH₂, (CH₂)₄N(CH₃)₂, (CH₂)₄N(CH₂CH₃)₂, (CH₂)₄N(CH₂CH₂CH₃)₂, (CH₂)₄N(CH(CH₃)₂)₂, (CH₂)₃NHC(═NH)NH₂, CH₂OH, CH(OH)CH₃, CH₂CH₂SCH₃, CH₂CH₂S(O₂)CH₃, CH₂SH, CH₂SO₃H, CH₂C₆H₅, and CH₂C₆H4OH;
R³⁴ is H, CH₃, CH₂CH₃, C(O)H, and C(O)CH₃; D1, D₂, D3 and D4 are cytotoxic drug and A₁, A₂ and A₃ are small molecular ligands or drugs m₁₁ is a number of 0, 1, 2, 3, 4, 5, 6, 7, 8, 9 and m₁2 is a number of 0, 1, 2, 3 and 4; when number is 0, it means the structure in the brackets can be omitted (crossed out);

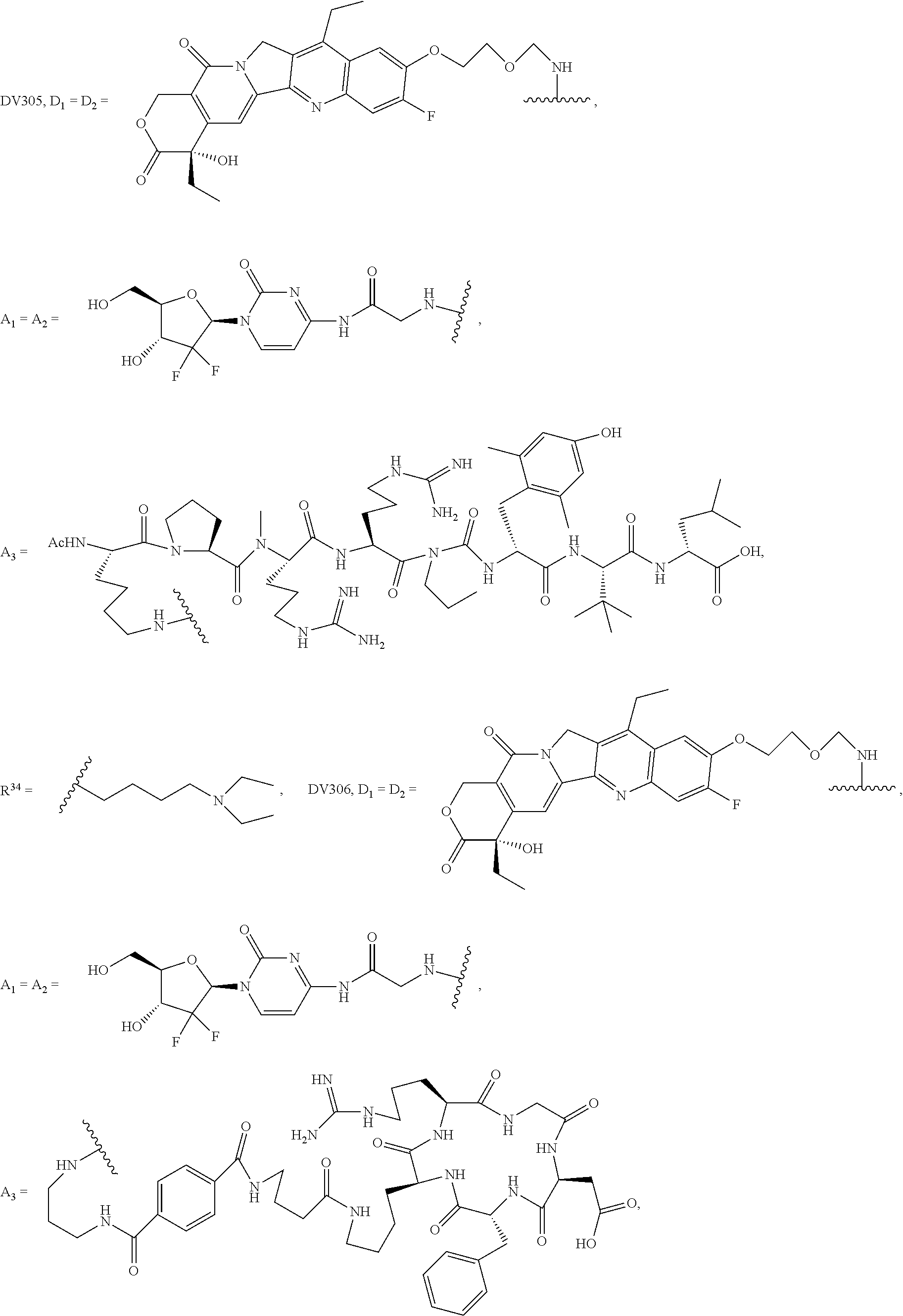

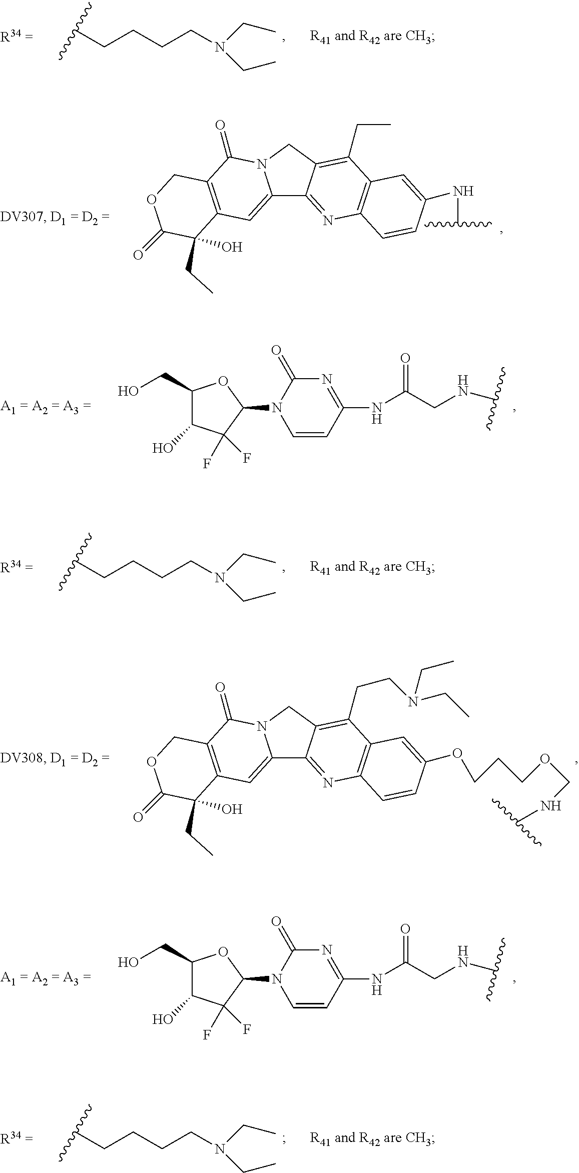

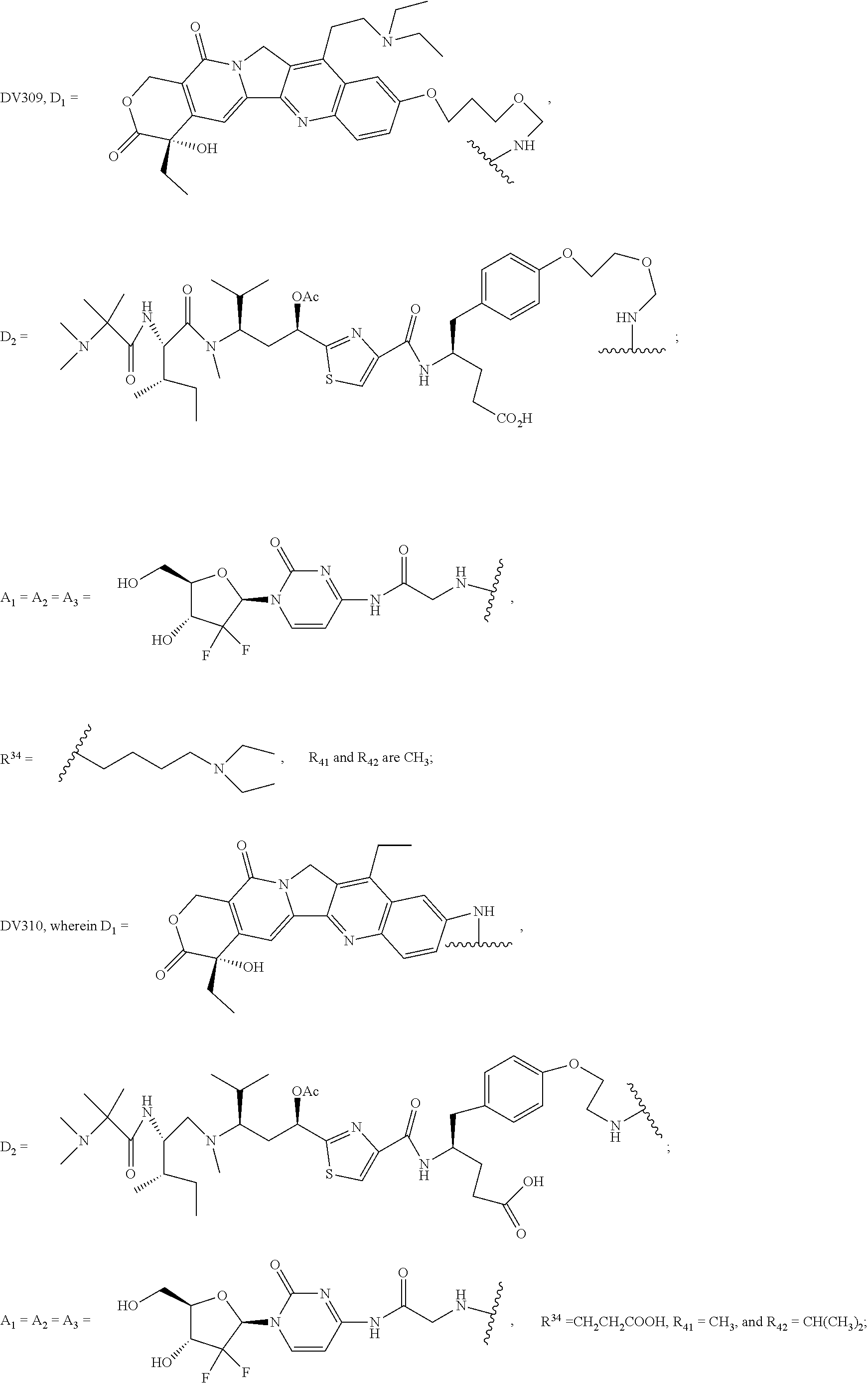

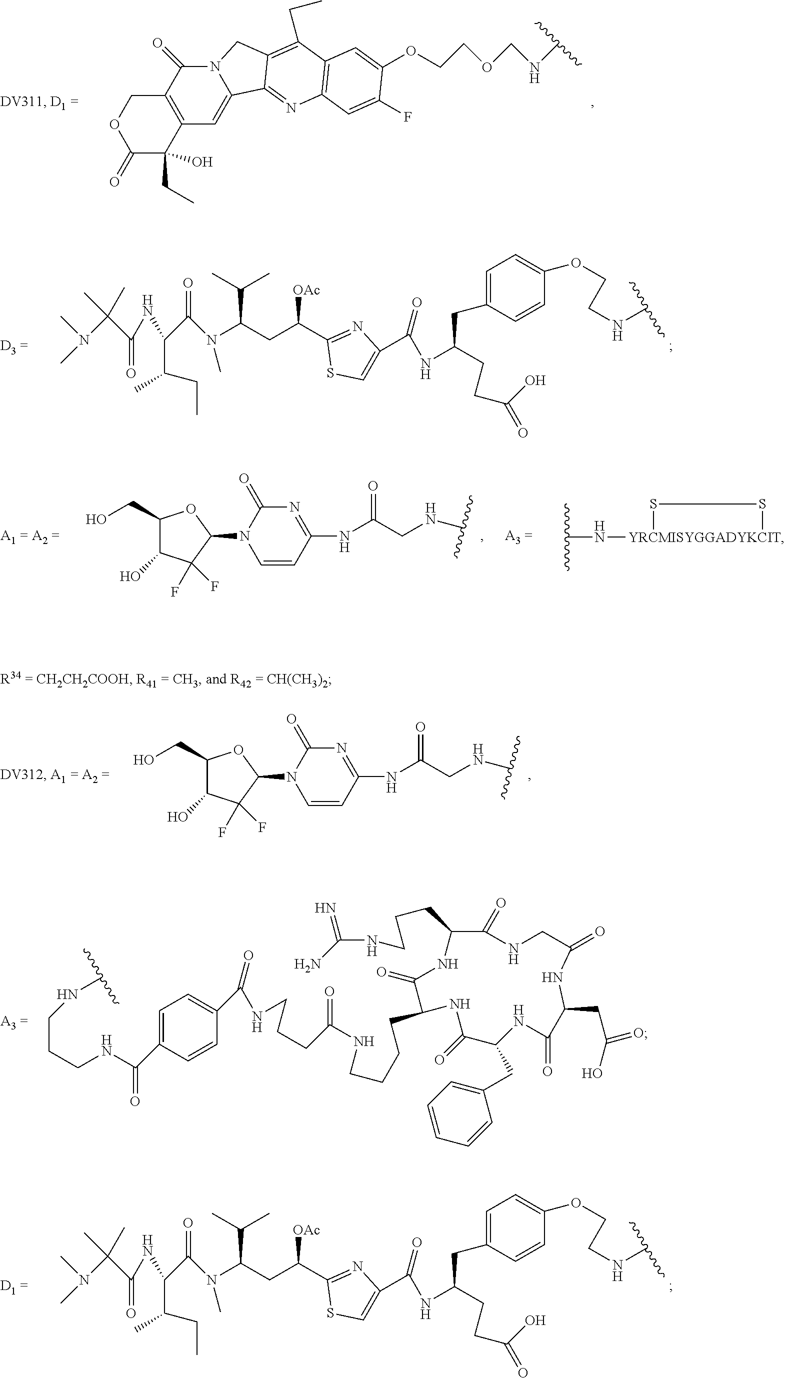

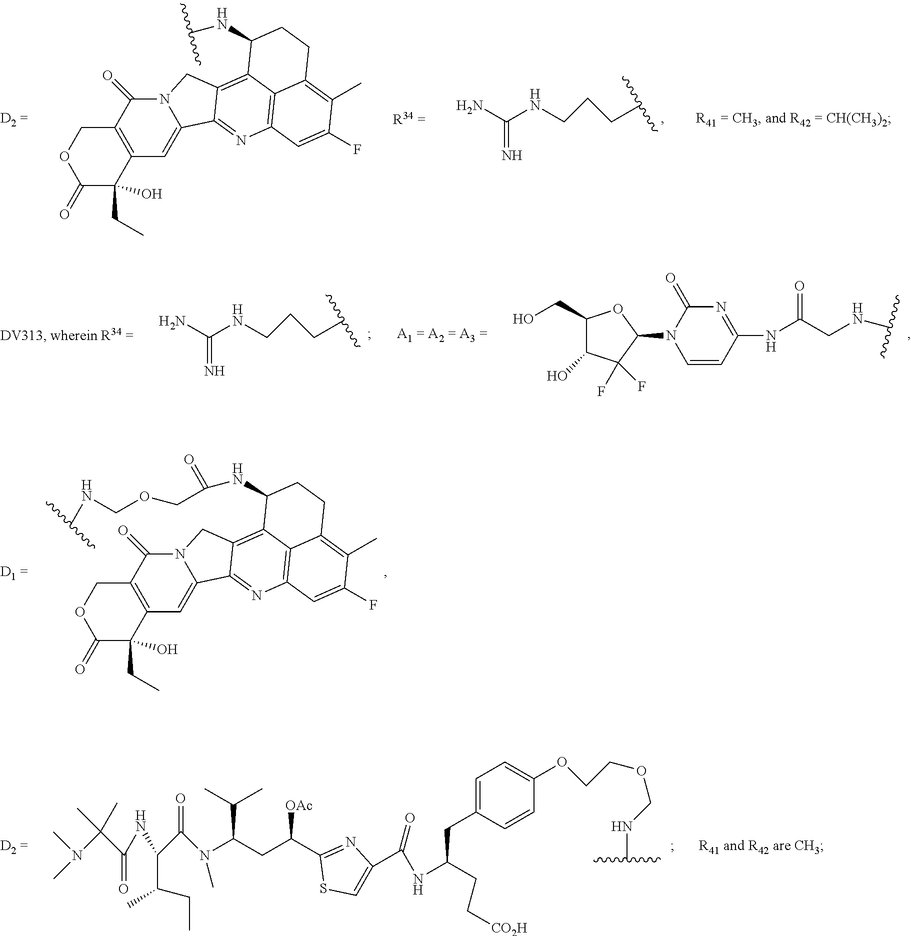 [SMILES: *N[C@H]1CCc2c(C)c(F)cc3nc4c(c1c23)Cn1c-4cc2c(c1=O)COC(=O)[C@]2(O)CC ; *CCCNC(=N)N ; *CCCNC(=N)N ; *NCC(=O)Nc1ccn([C@@H]2O[C@H](CO)[C@@H](O)C2(F)F)c(=O)n1 ; *NCOCC(=O)N[C@H]1CCc2c(C)c(F)cc3nc4c(c1c23)Cn1c-4cc2c(c1=O)COC(=O)[C@]2(O)CC ; *NCOCCOc1ccc(C[C@@H](CCC(=O)O)NC(=O)c2csc([C@@H](C[C@H](C(C)C)N(C)C(=O)[C@@H](NC(=O)C(C)(C)N(C)C)[C@@H](C)CC)OC(C)=O)n2)cc1]

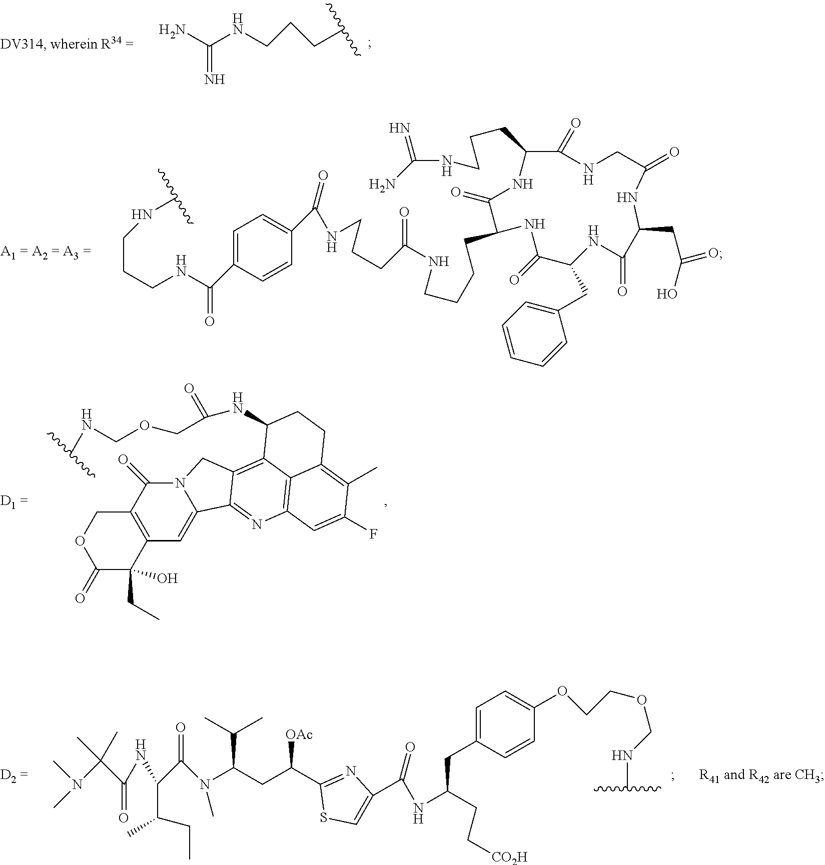 [SMILES: *CCCNC(=N)N ; *NCCCNC(=O)c1ccc(C(=O)NCCCC(=O)NCCCC[C@@H]2NC(=O)[C@@H](Cc3ccccc3)NC(=O)[C@H](CC(=O)O)NC(=O)CNC(=O)[C@H](CCCNC(=N)N)NC2=O)cc1 ; *NCOCC(=O)N[C@H]1CCc2c(C)c(F)cc3nc4c(c1c23)Cn1c-4cc2c(c1=O)COC(=O)[C@]2(O)CC ; *NCOCCOc1ccc(C[C@@H](CCC(=O)O)NC(=O)c2csc([C@@H](C[C@H](C(C)C)N(C)C(=O)[C@@H](NC(=O)C(C)(C)N(C)C)[C@@H](C)CC)OC(C)=O)n2)cc1]

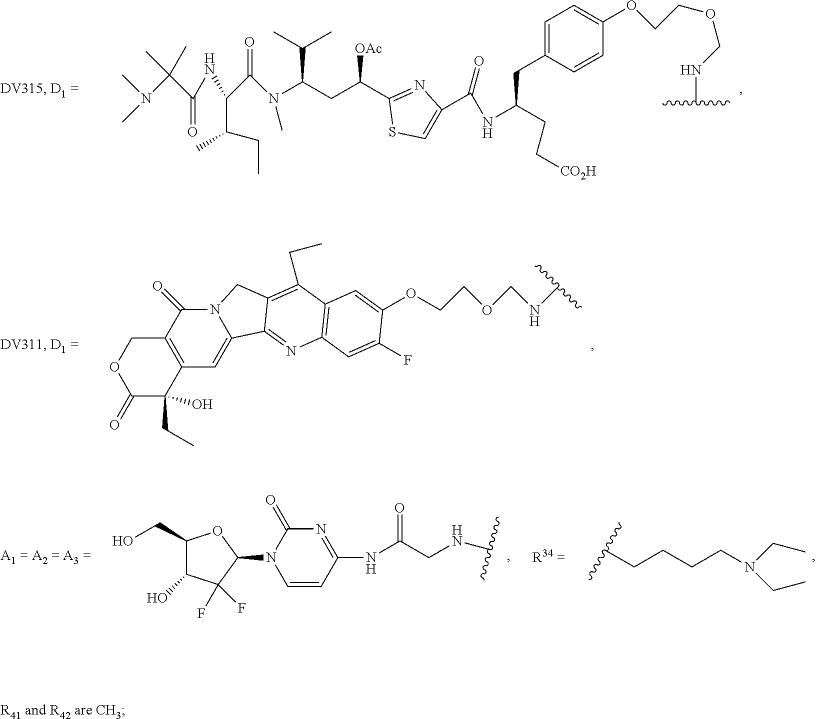

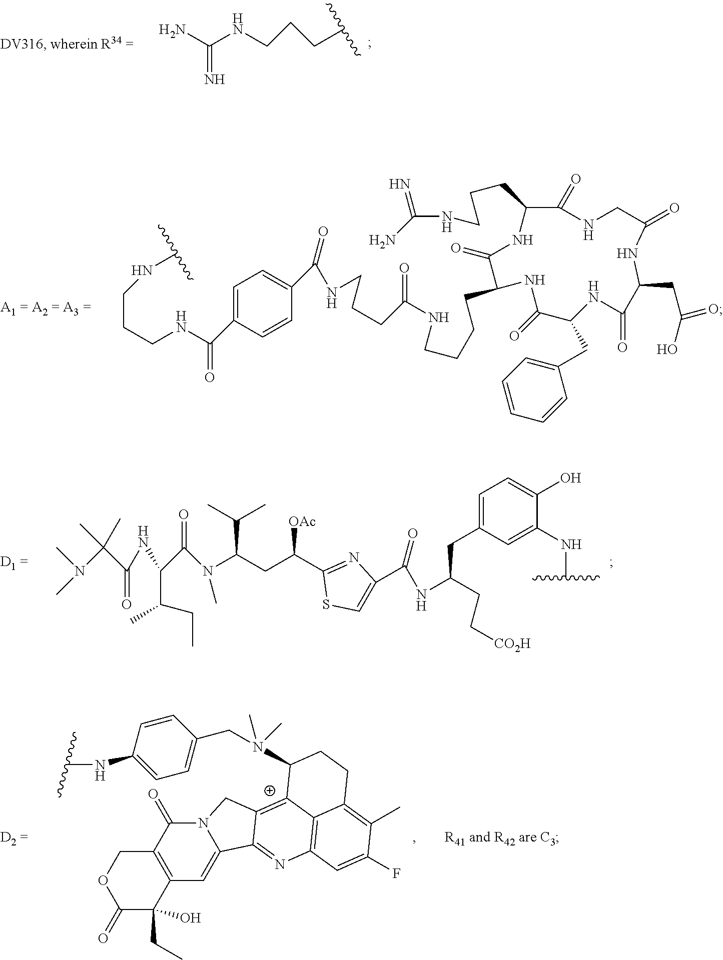

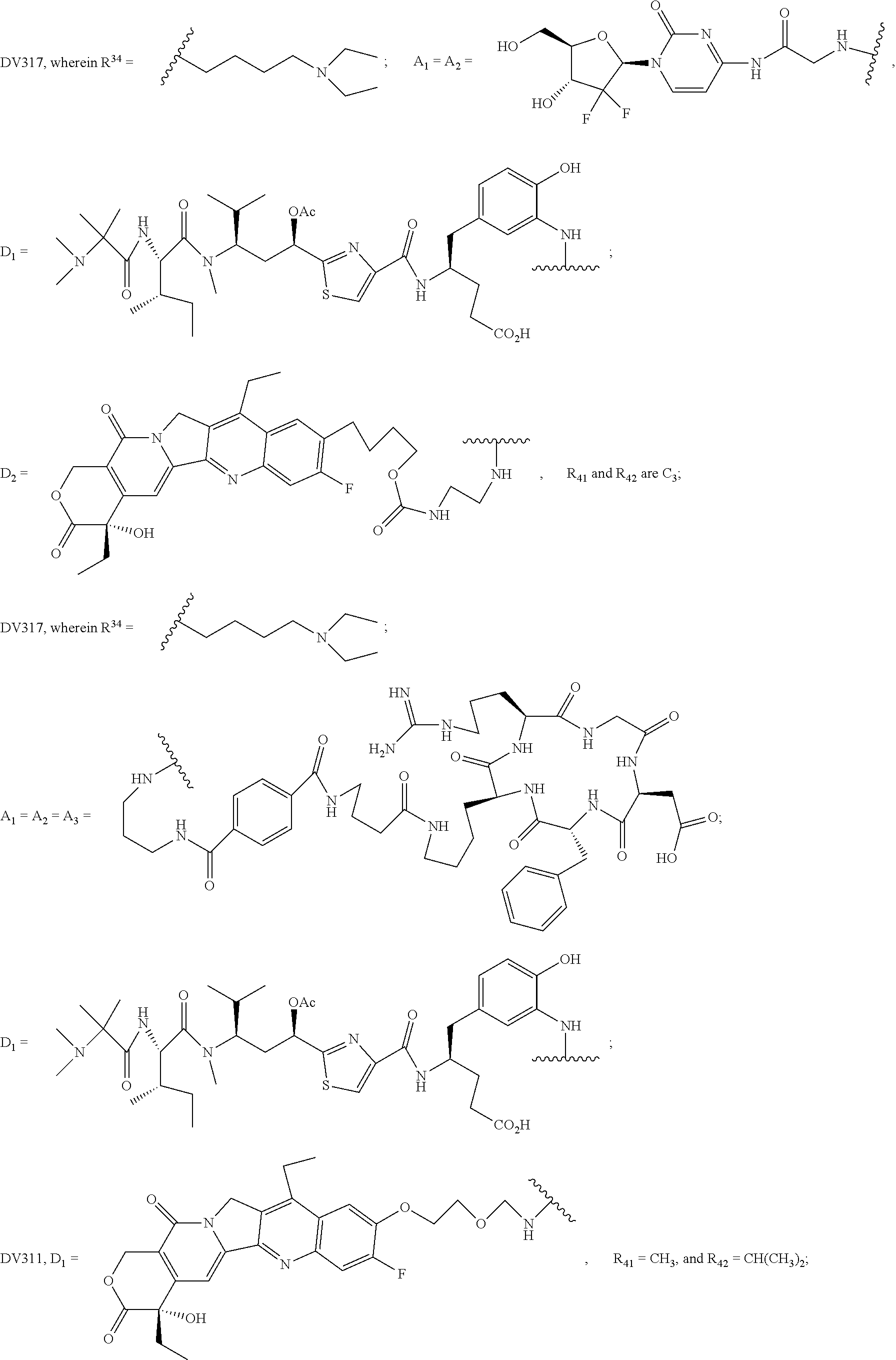

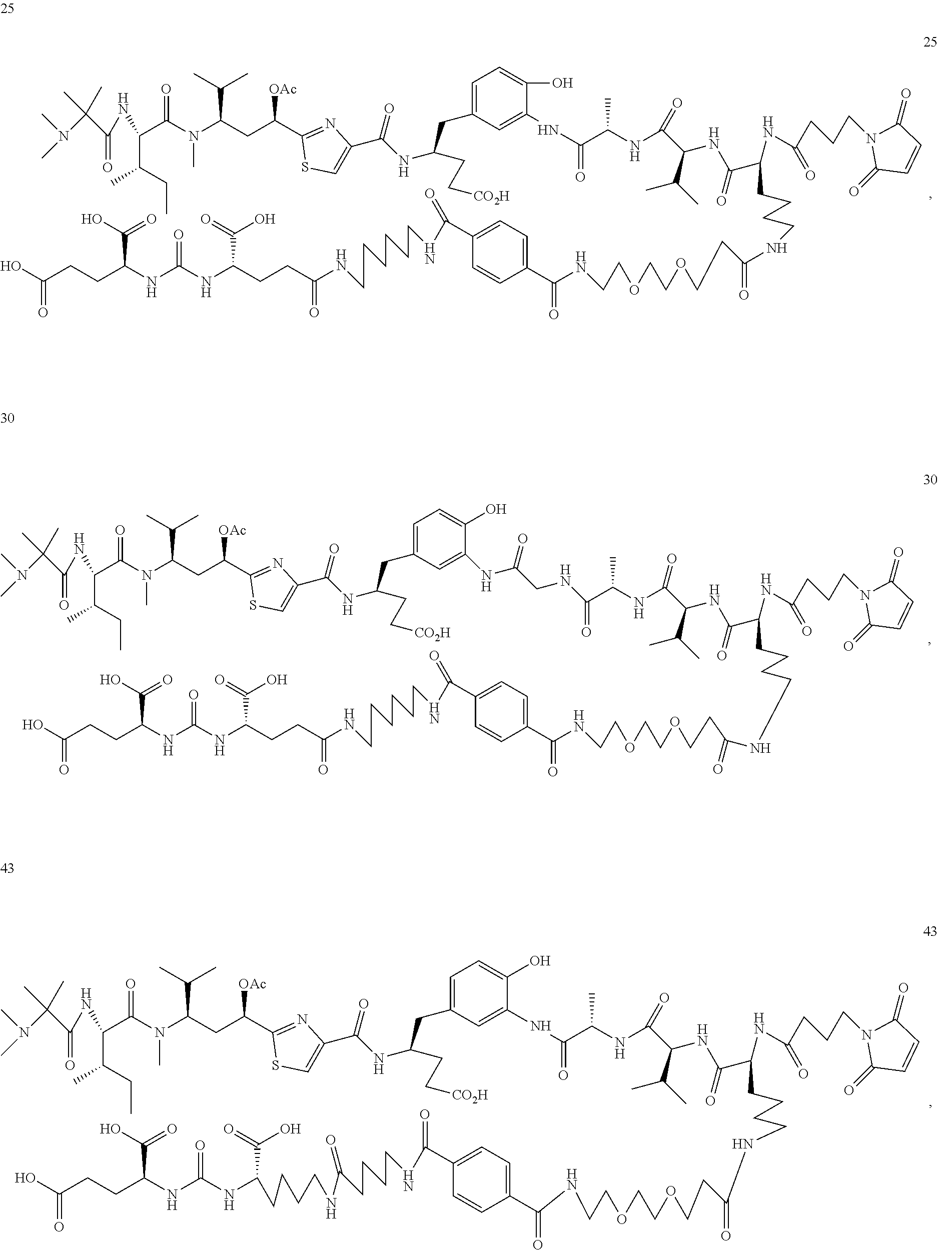

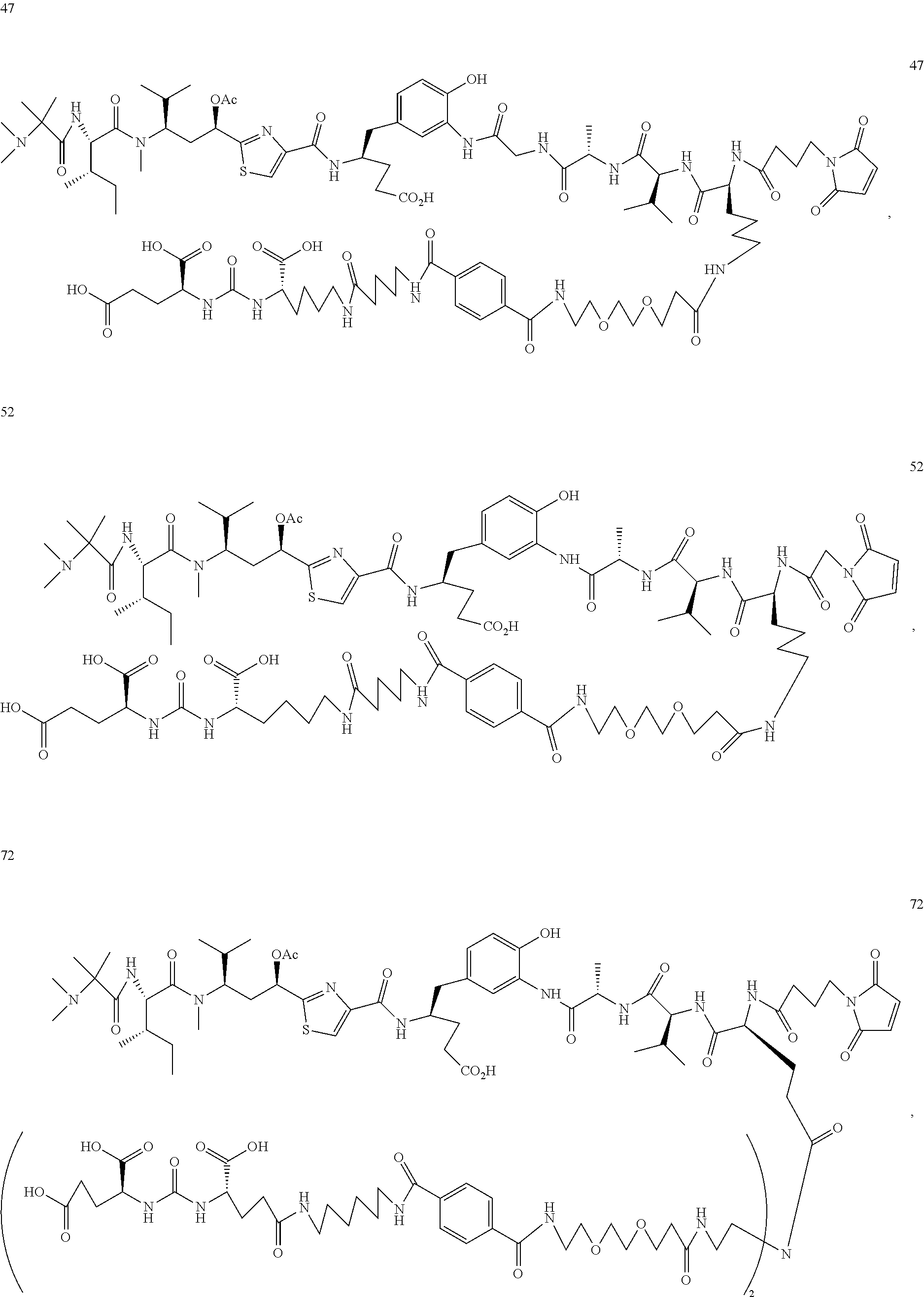

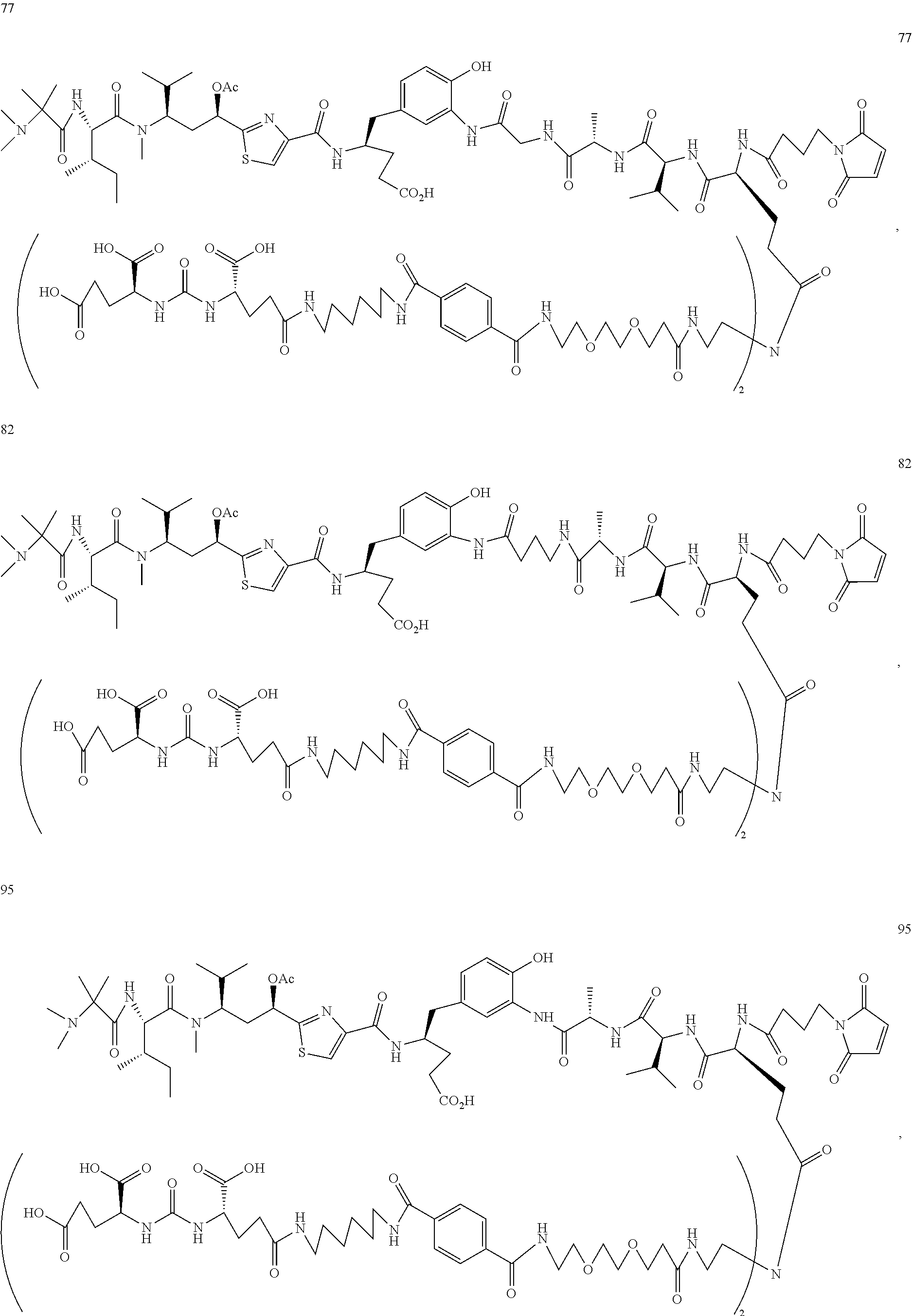

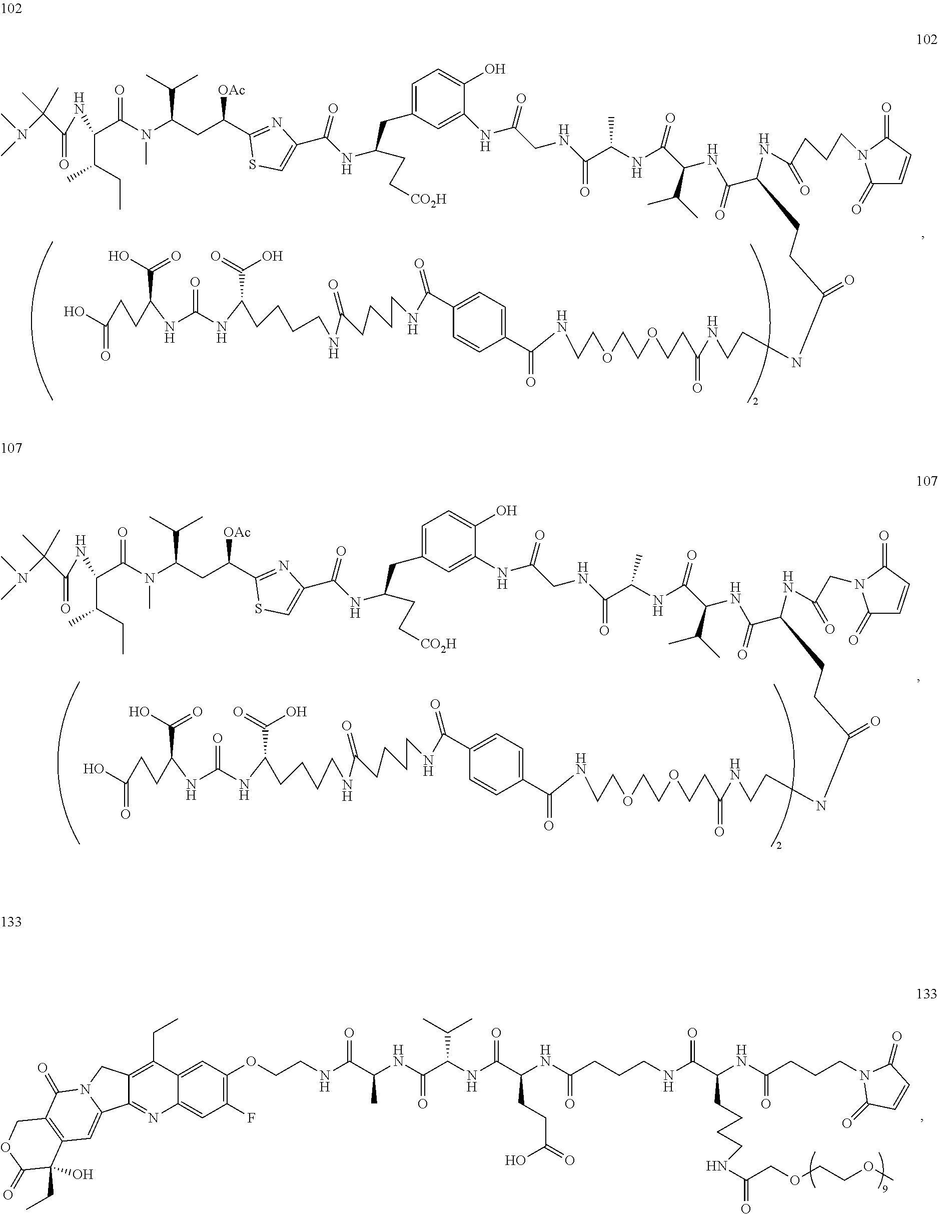

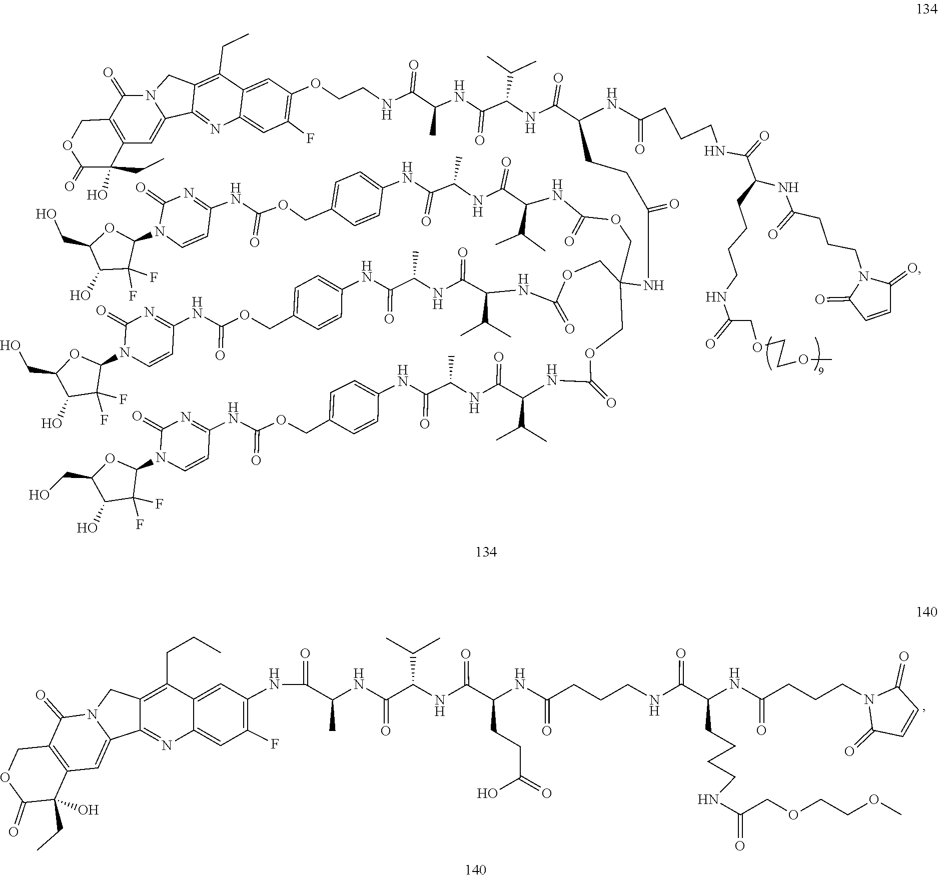

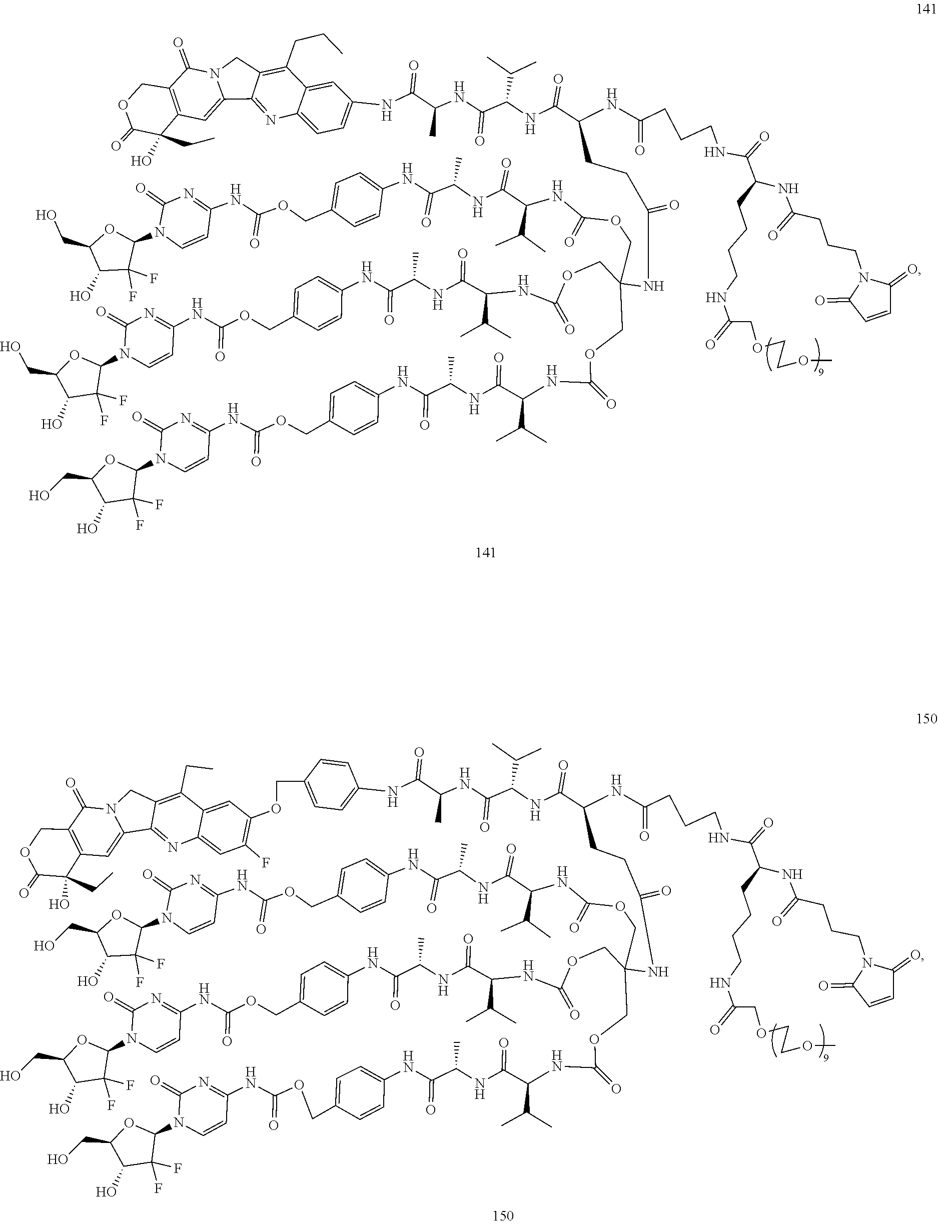

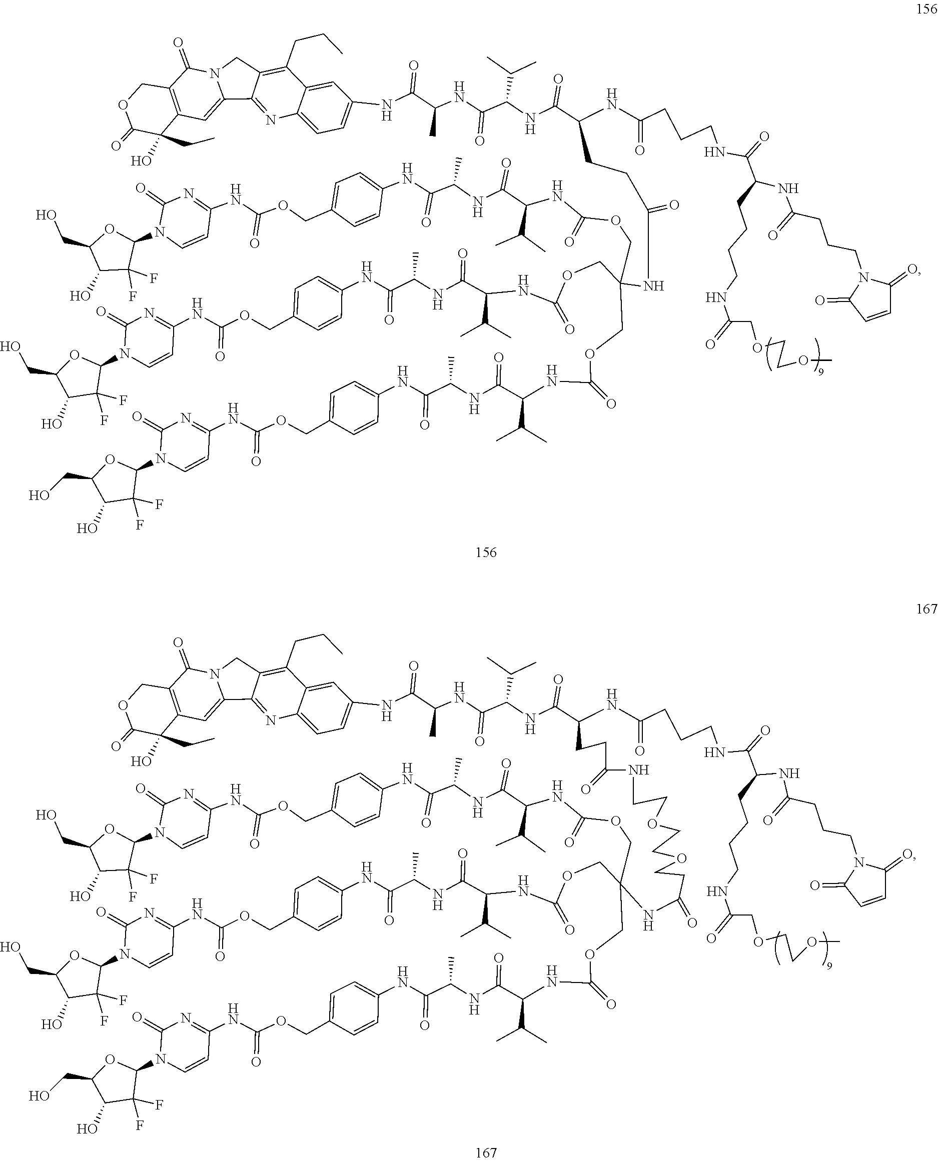

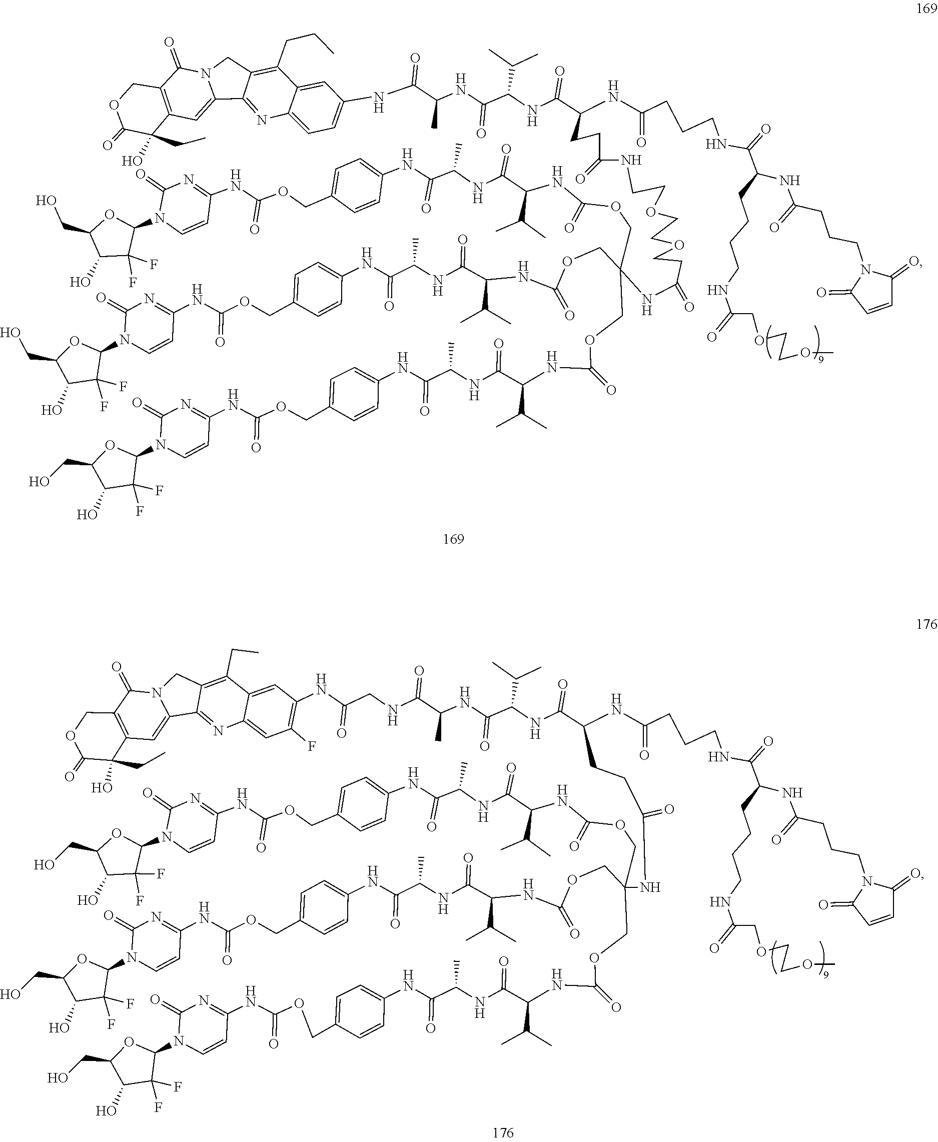

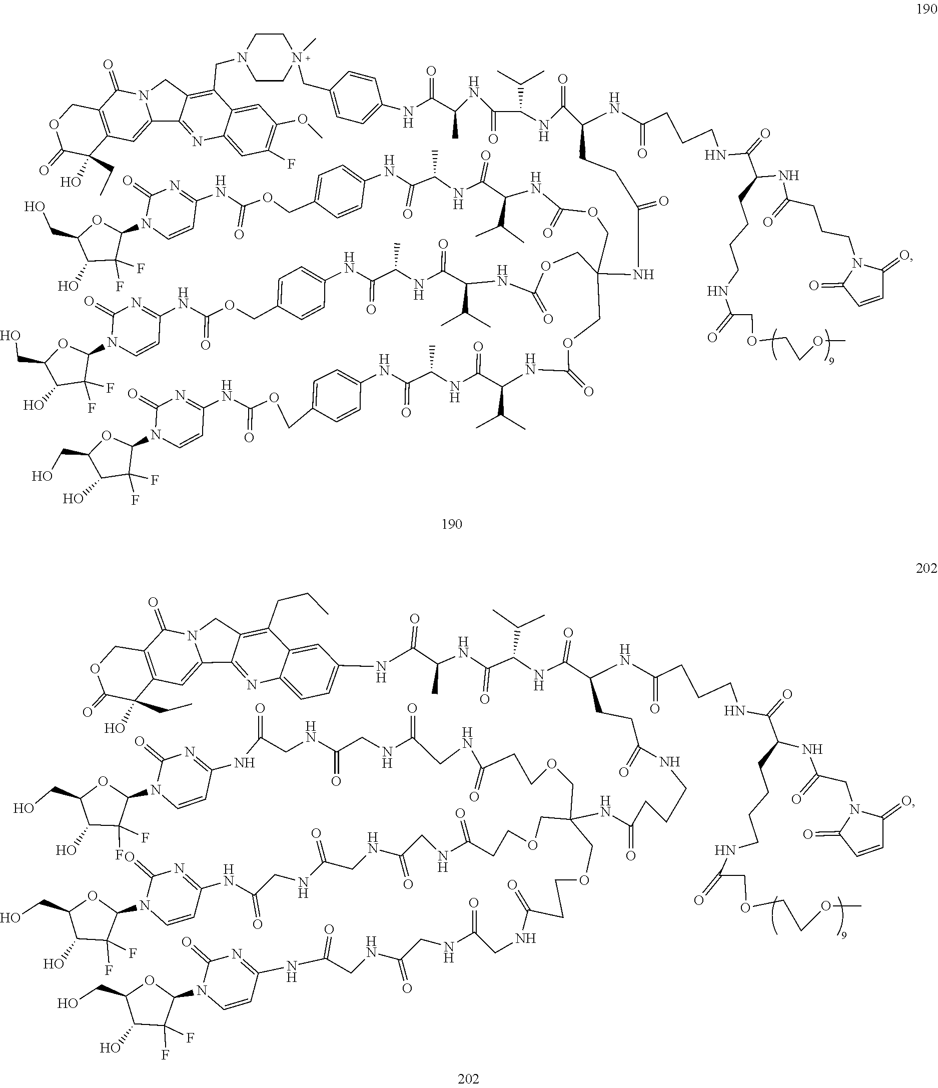

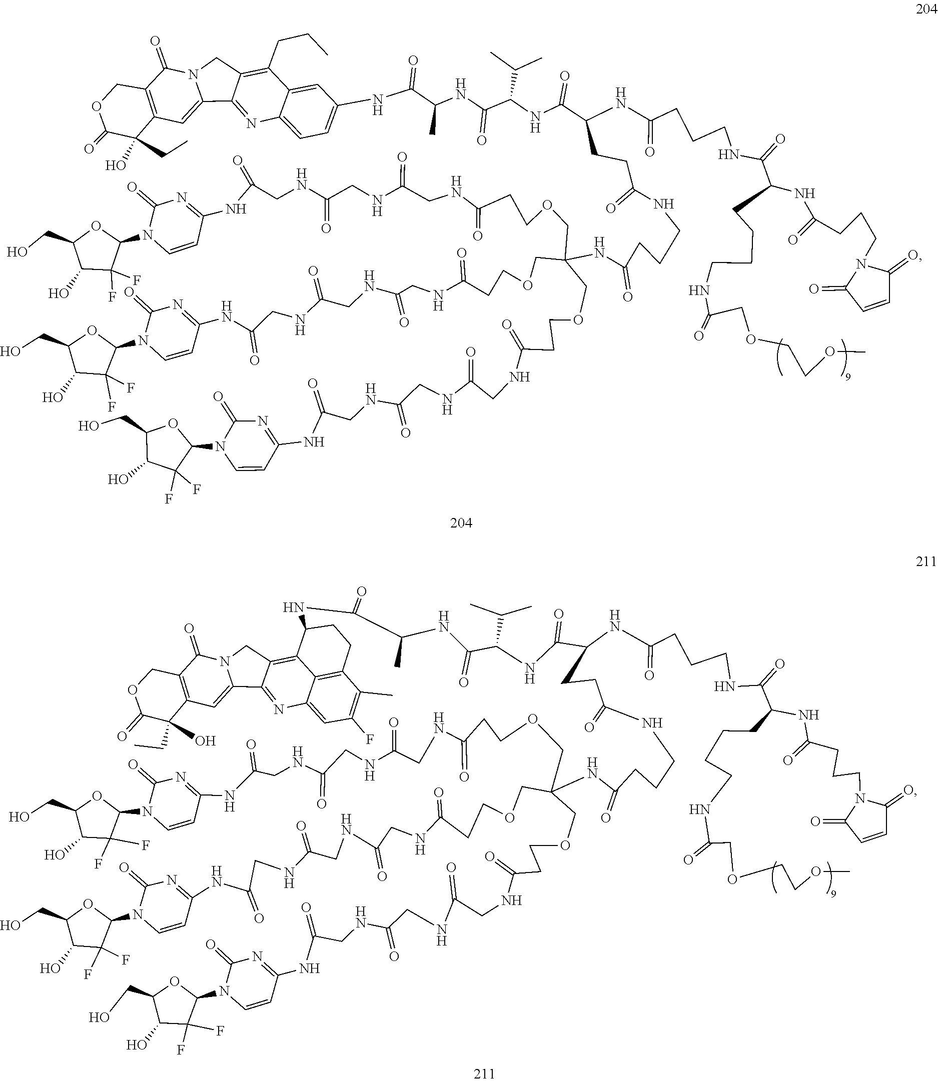

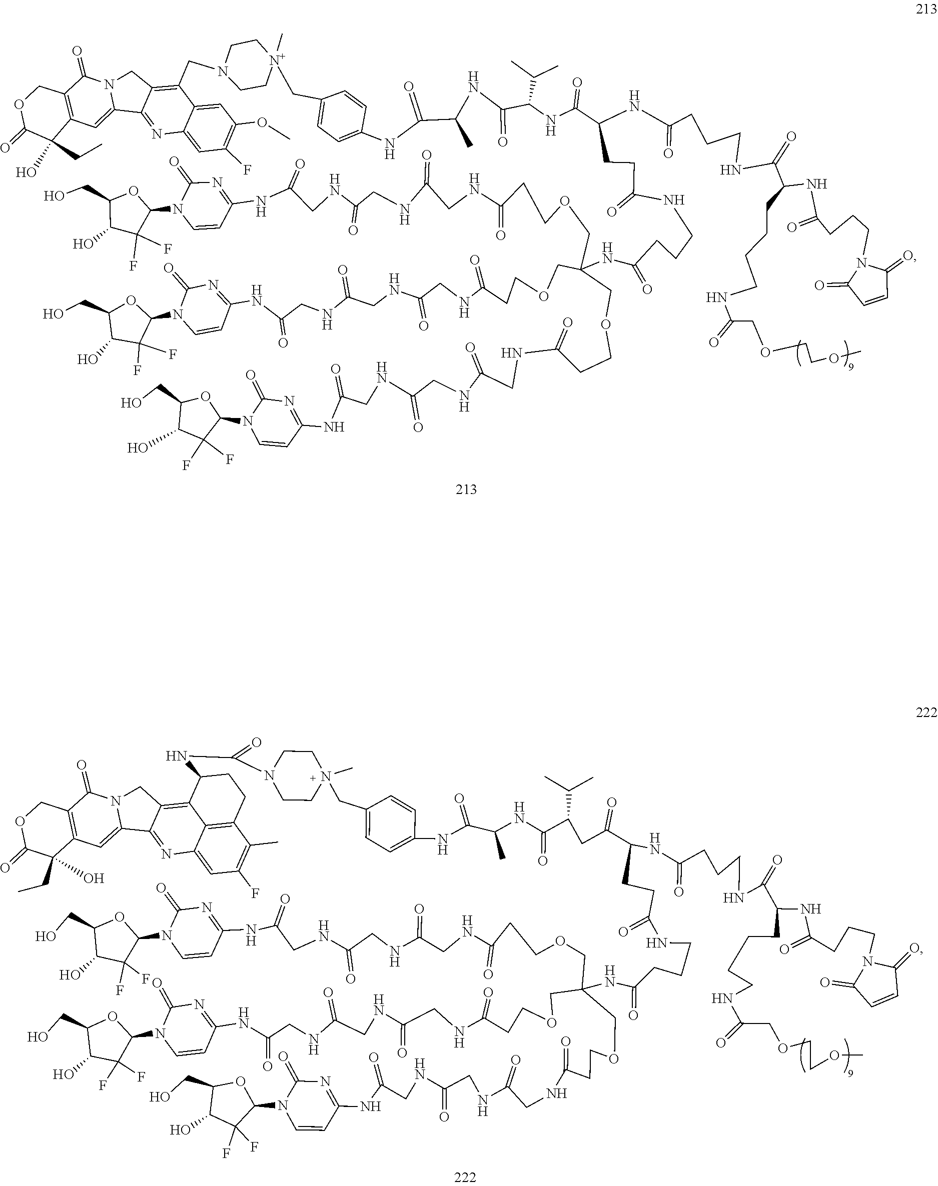

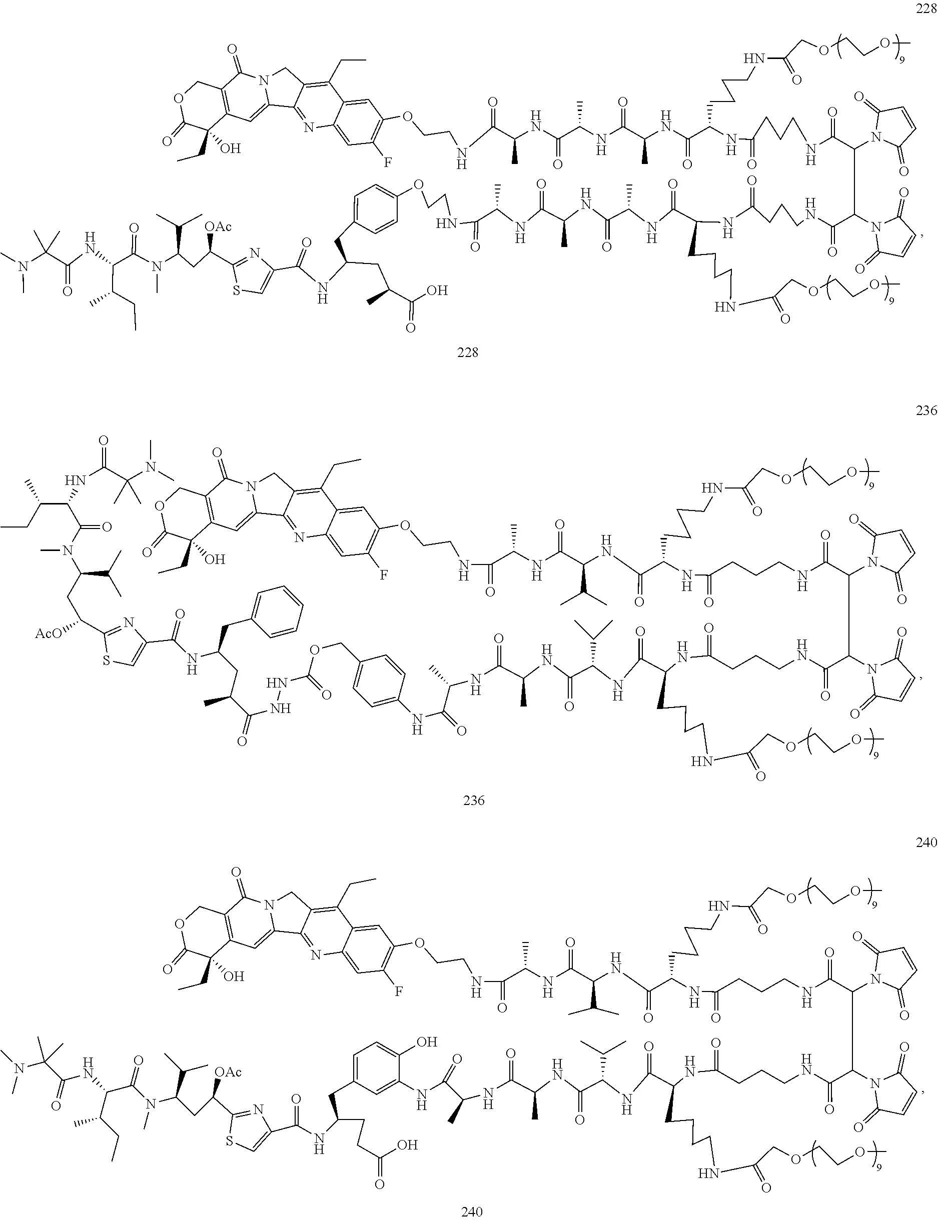

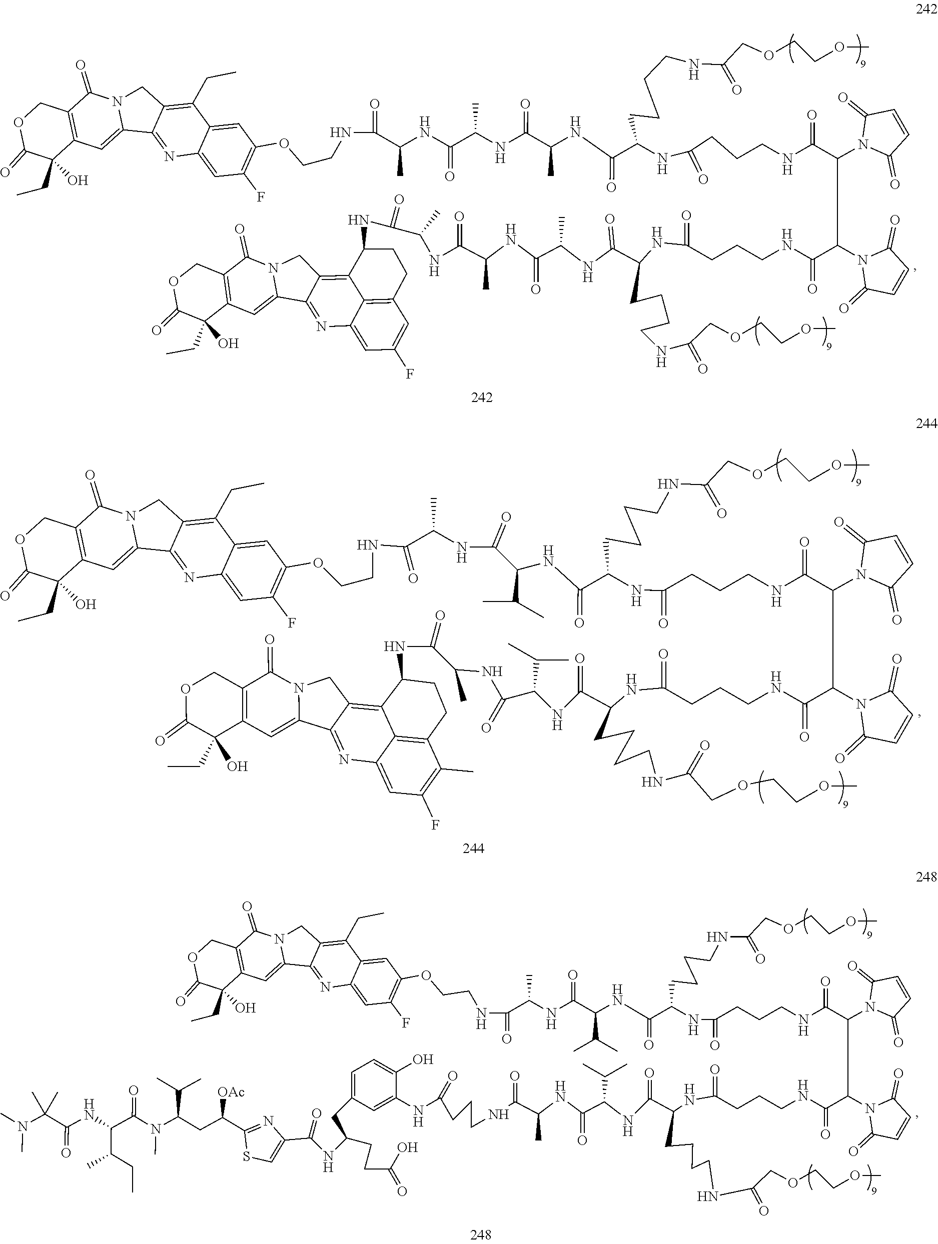

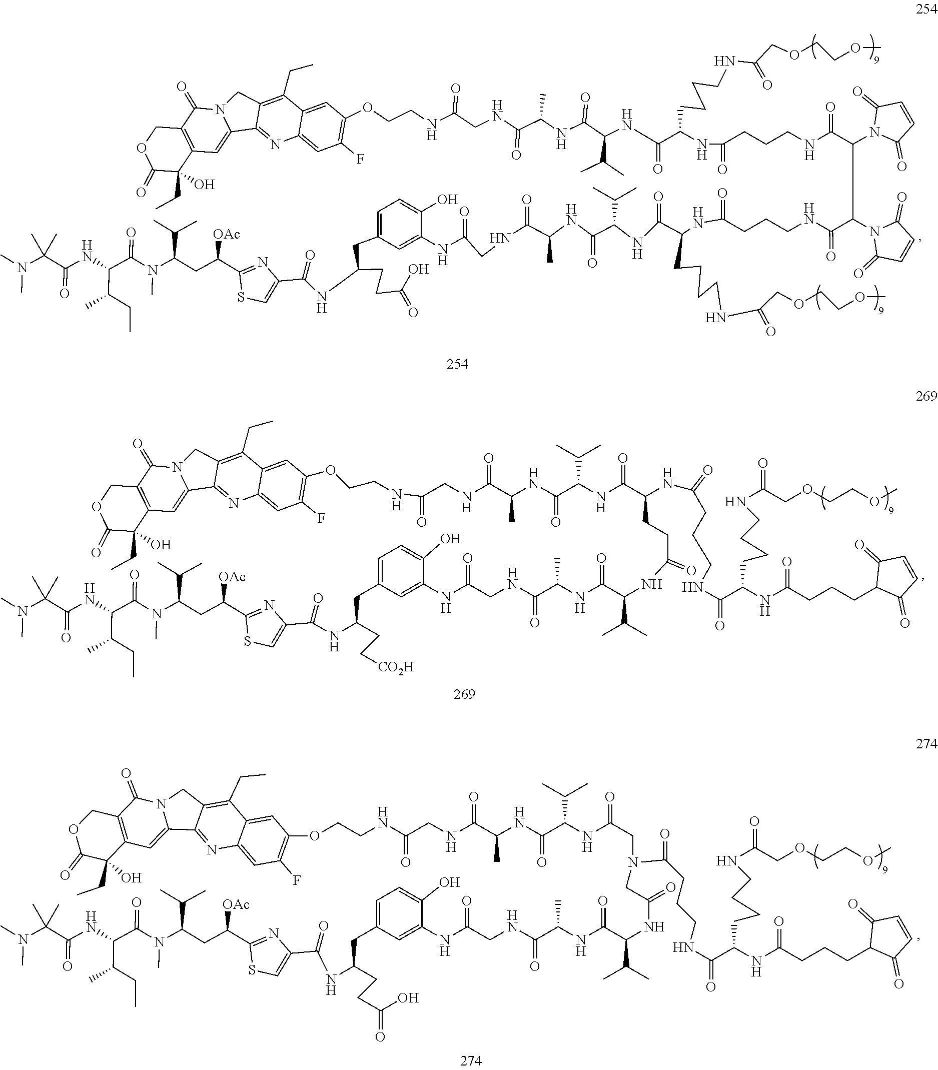

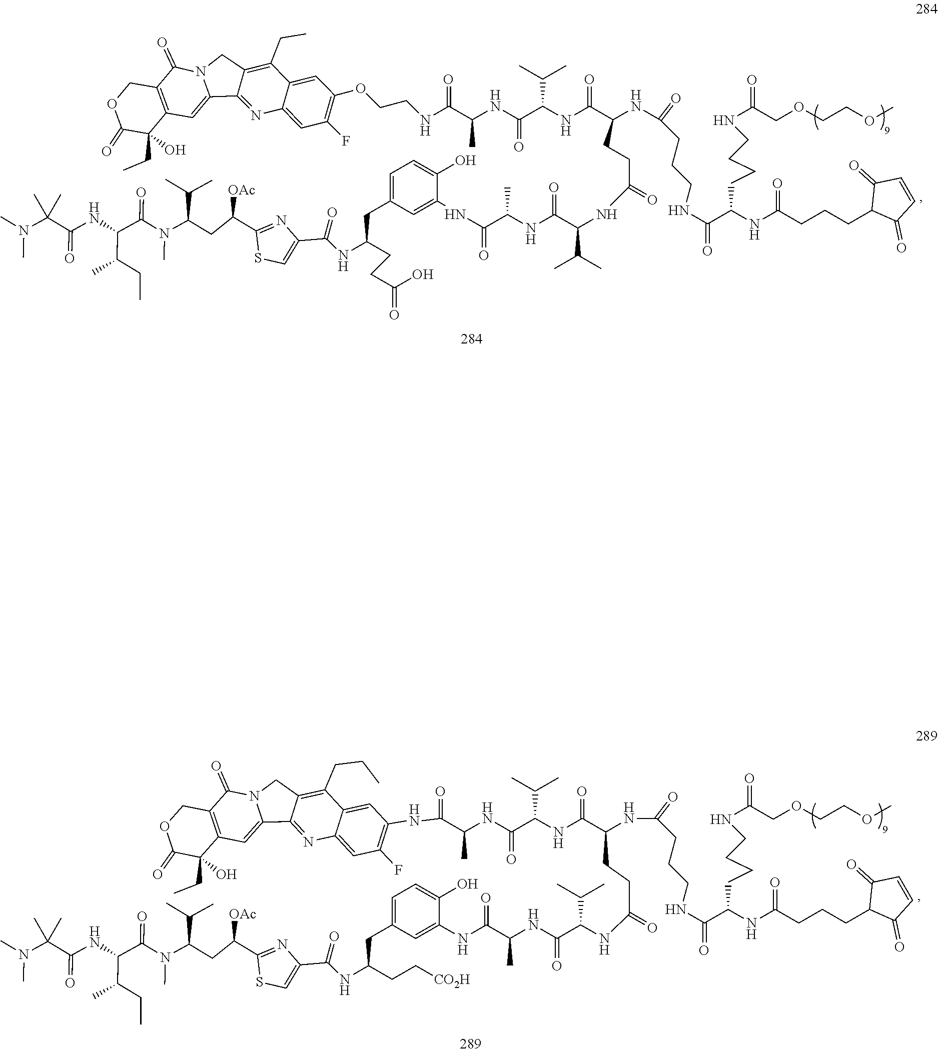

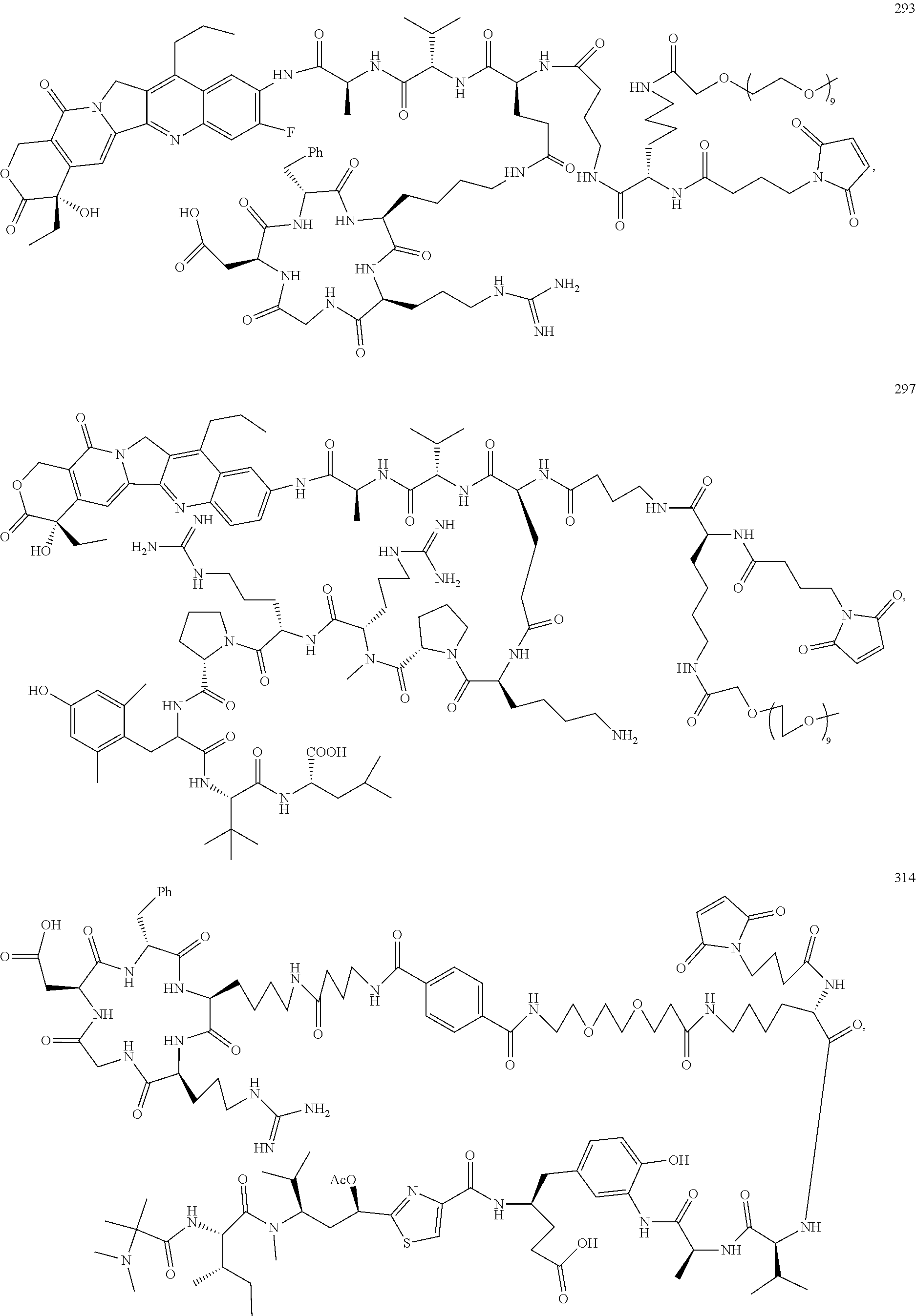

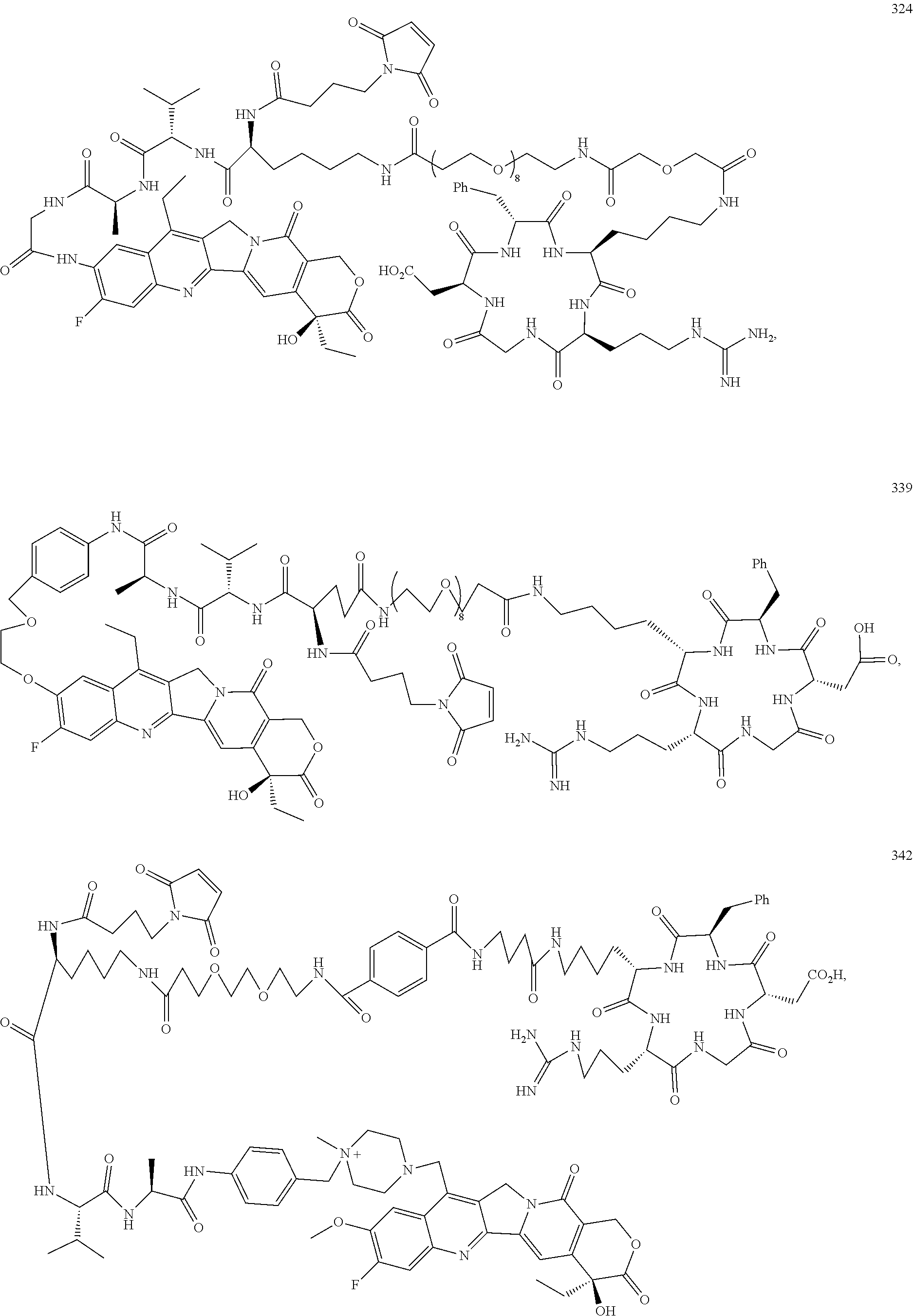

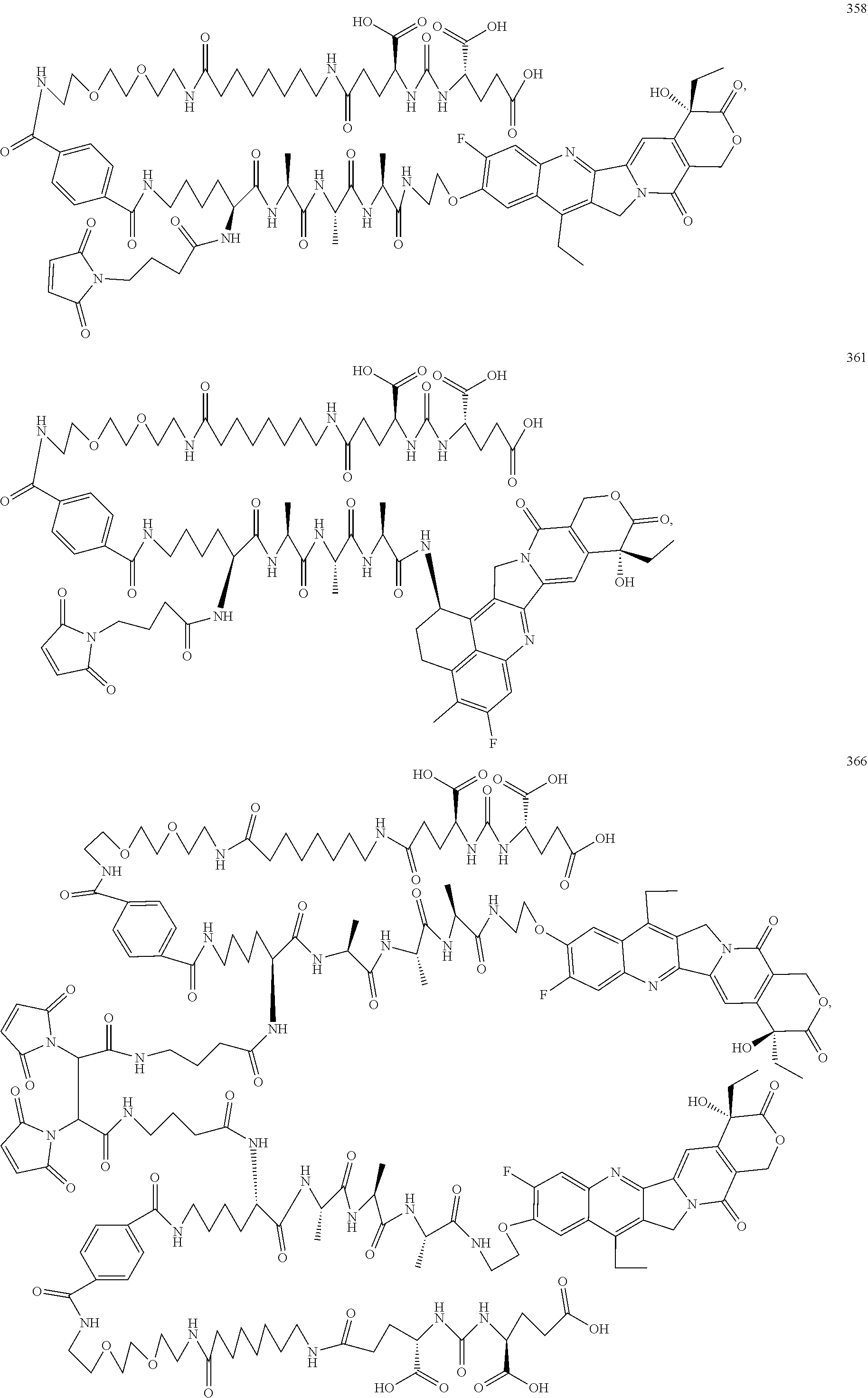

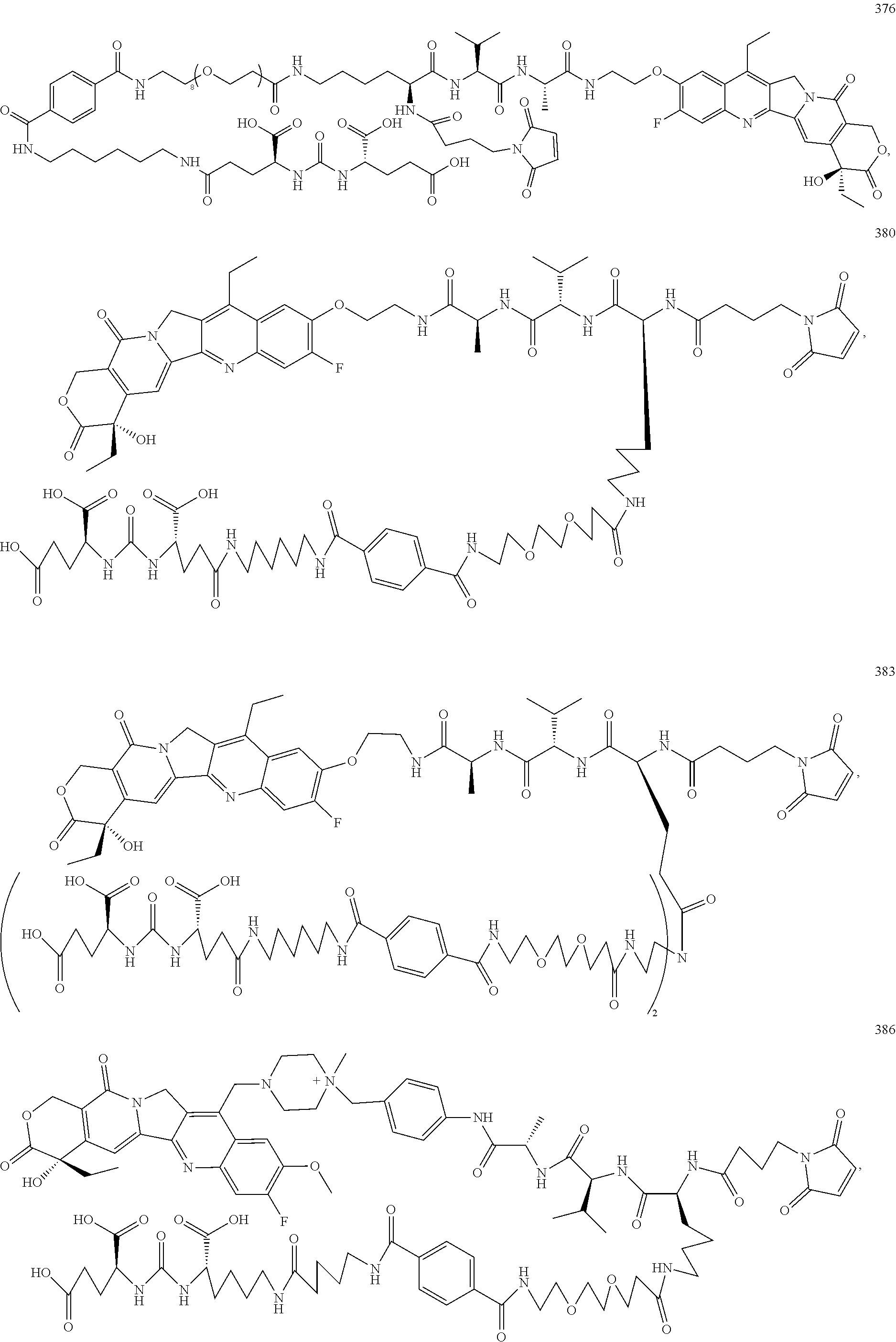

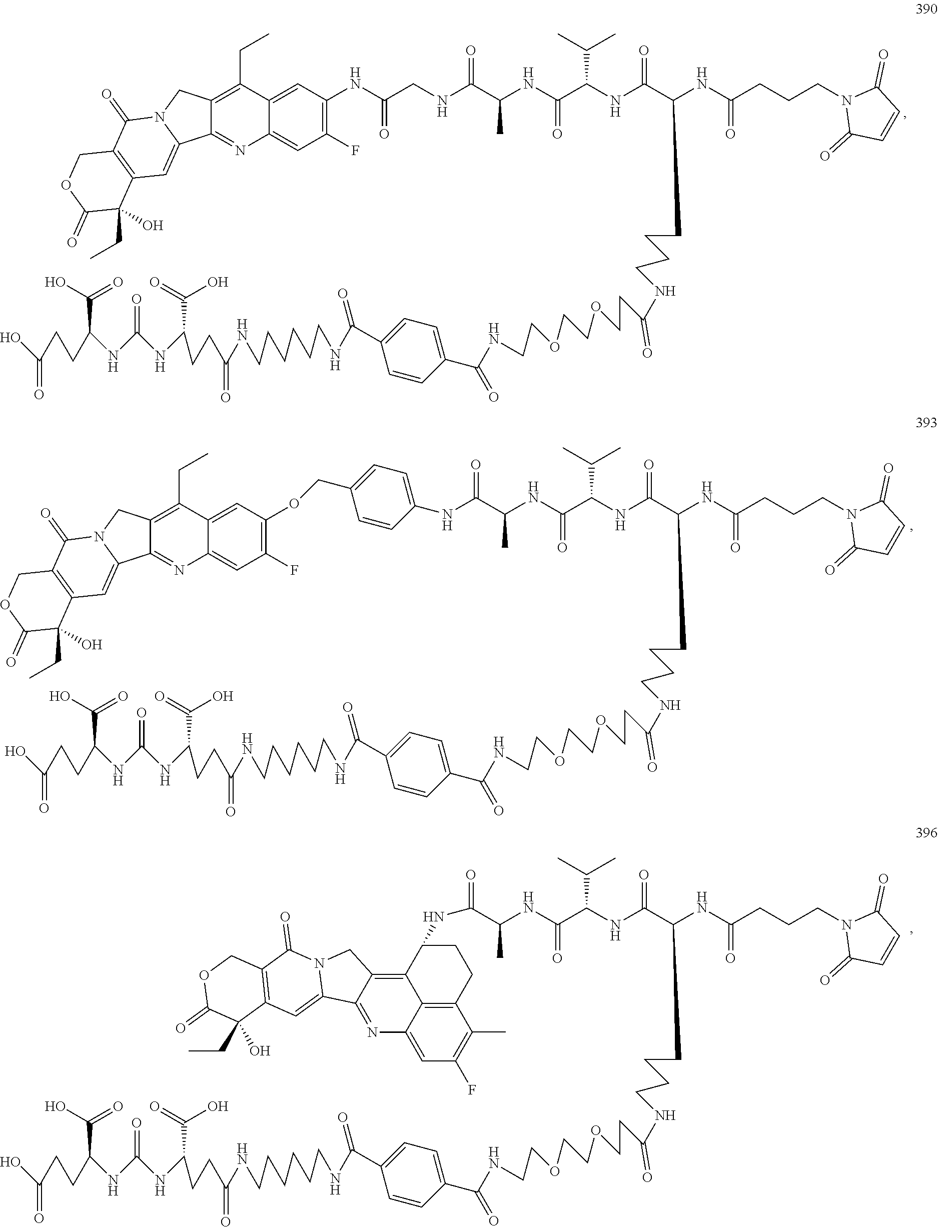

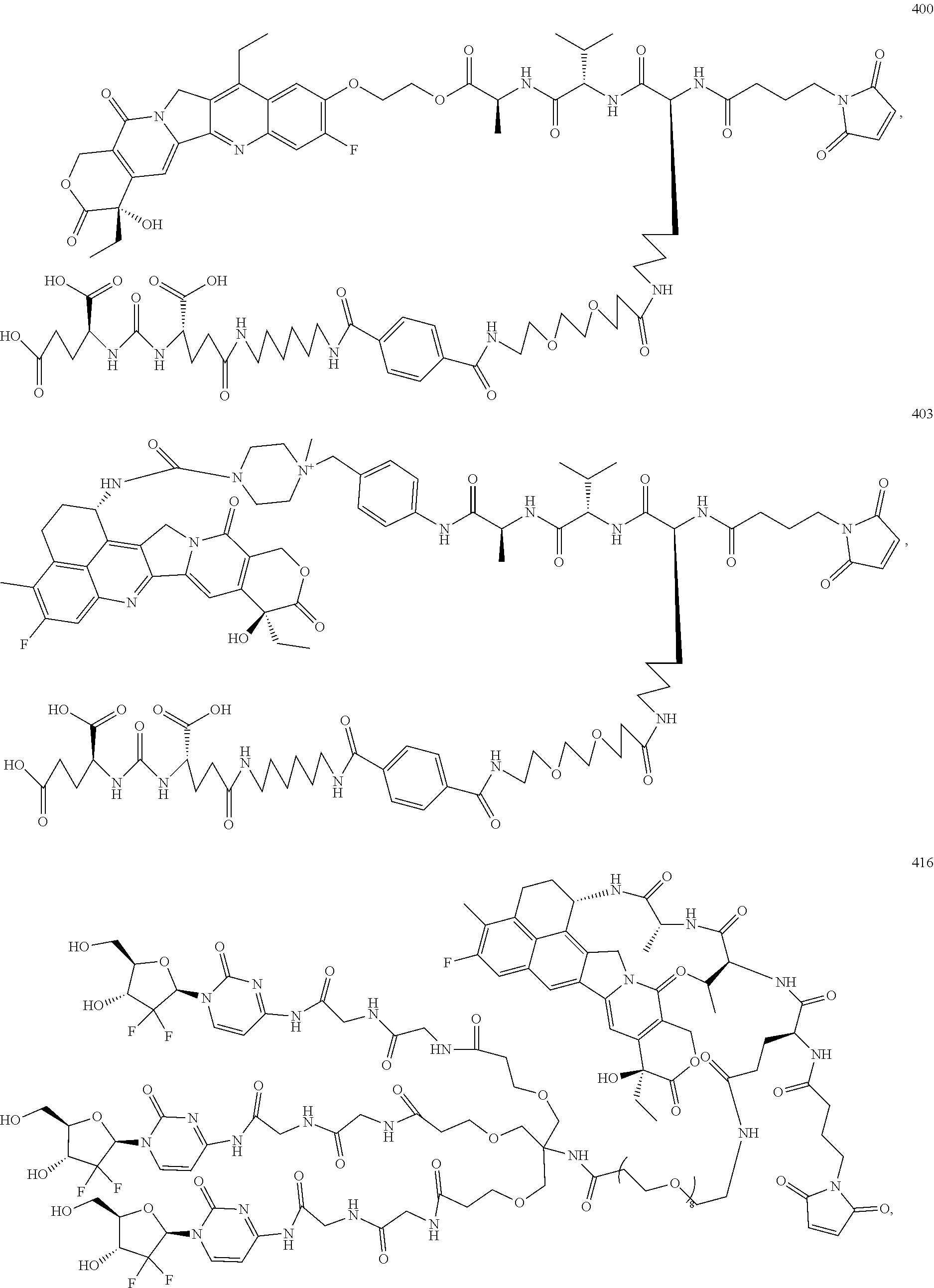

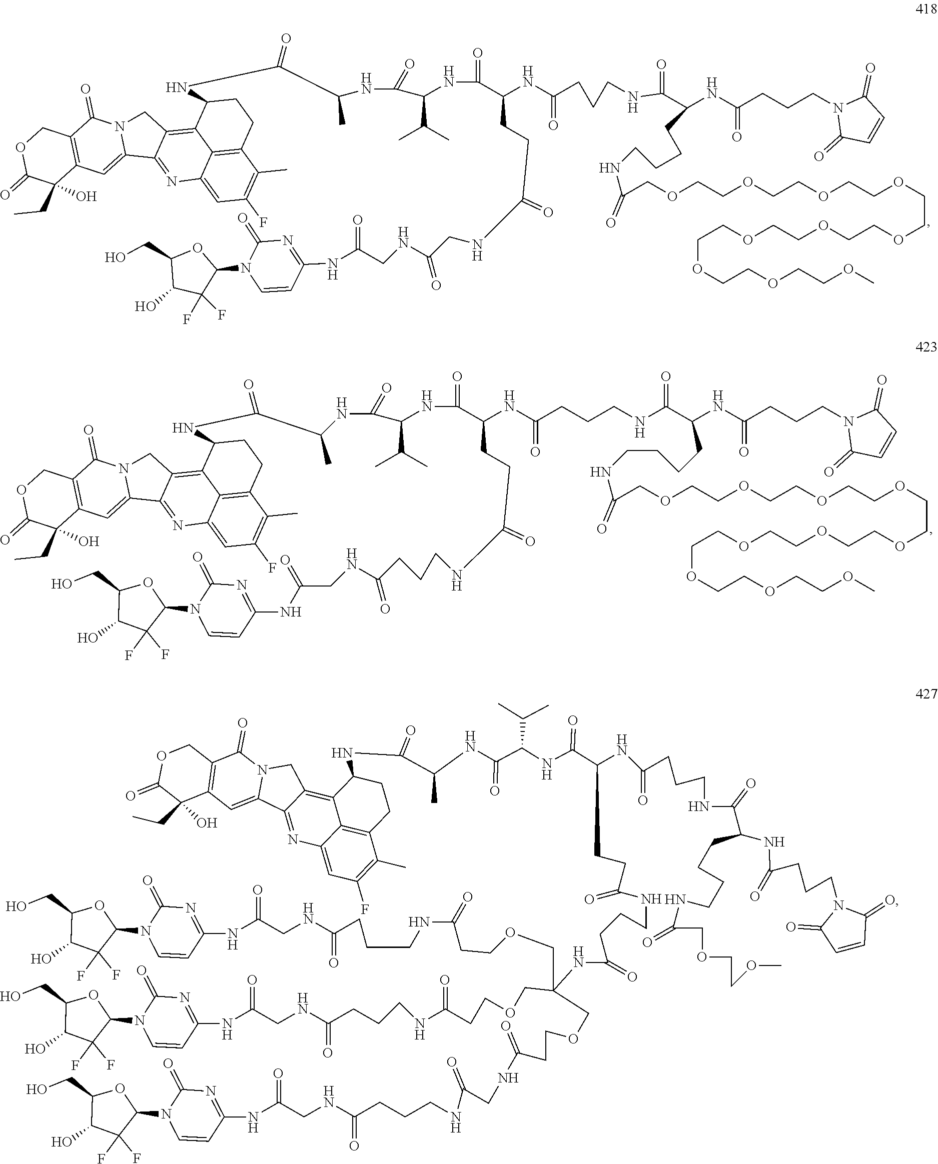

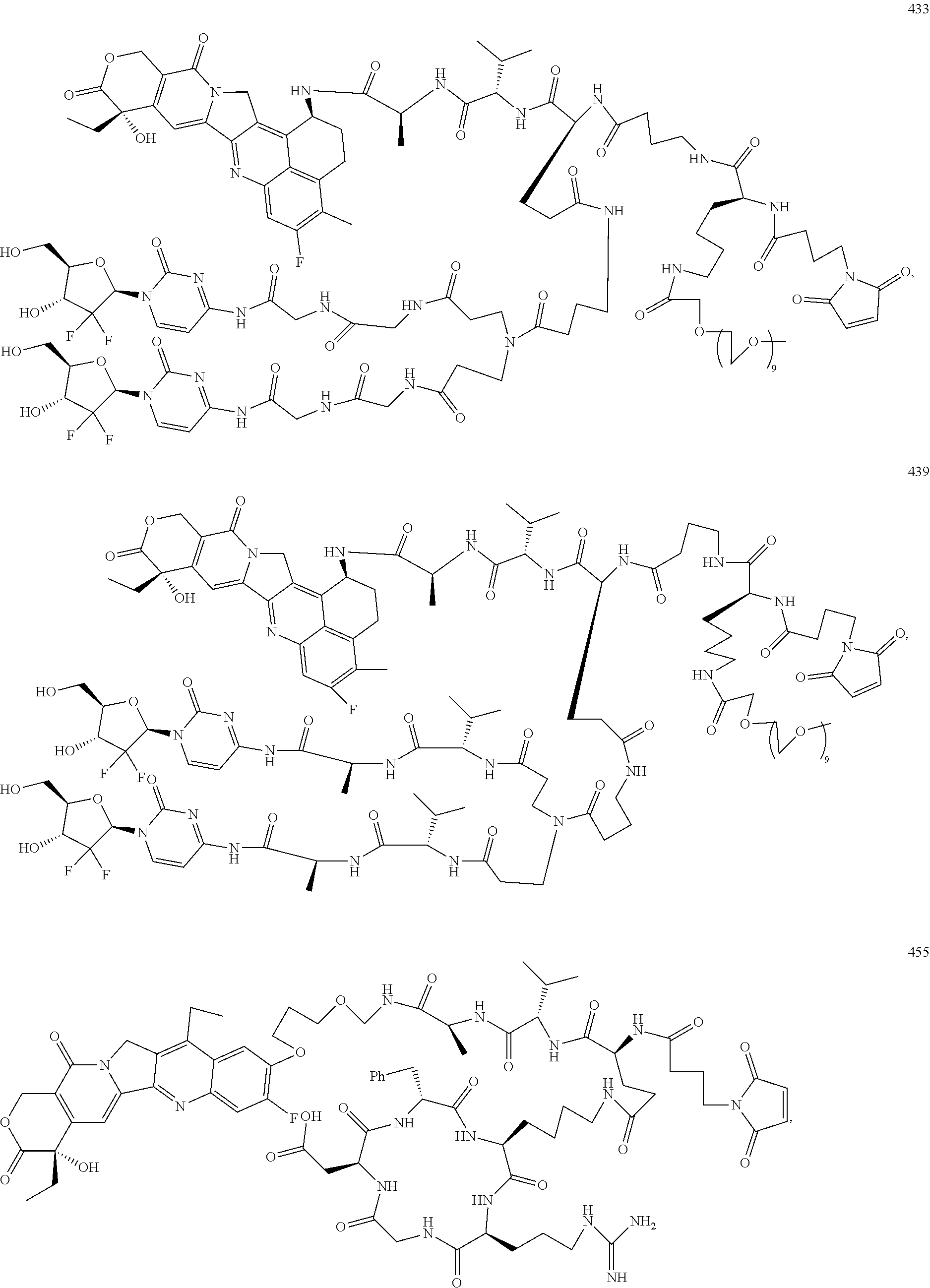

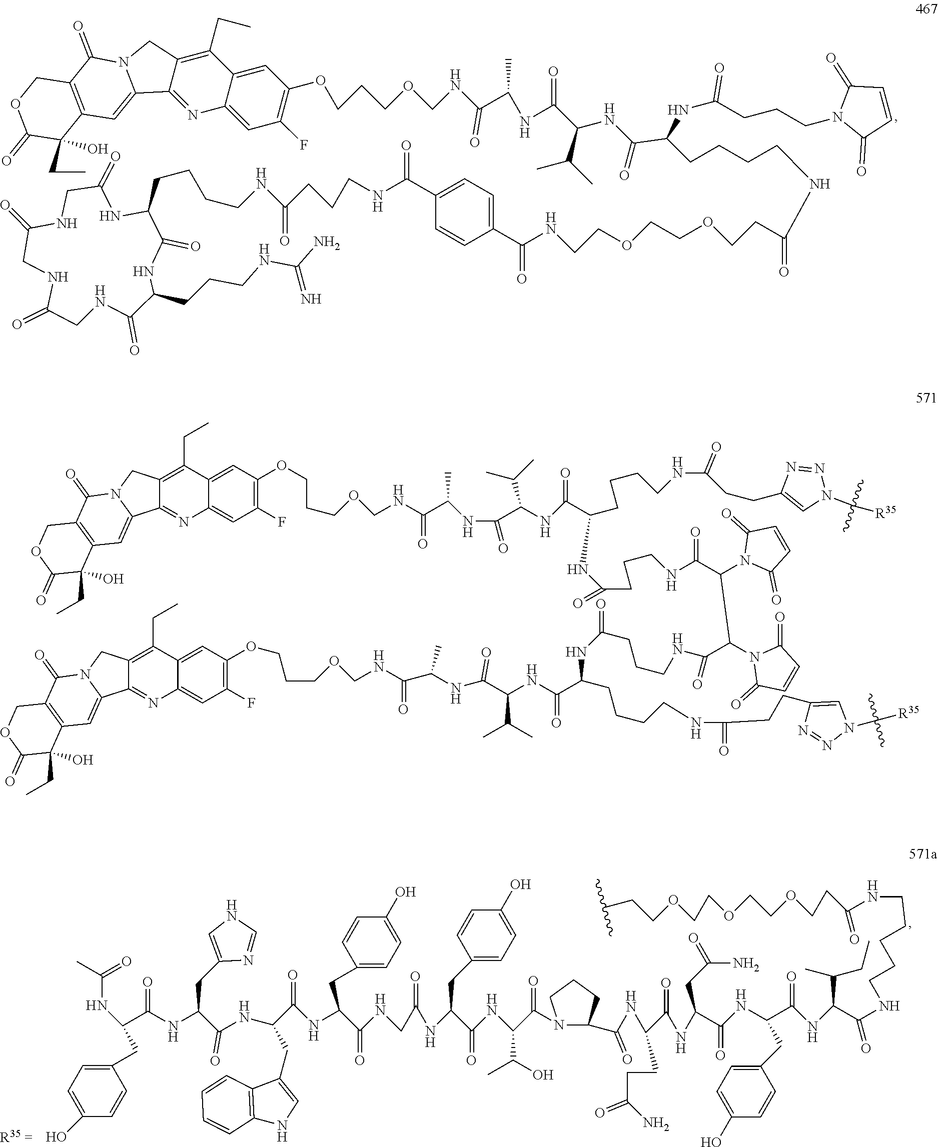

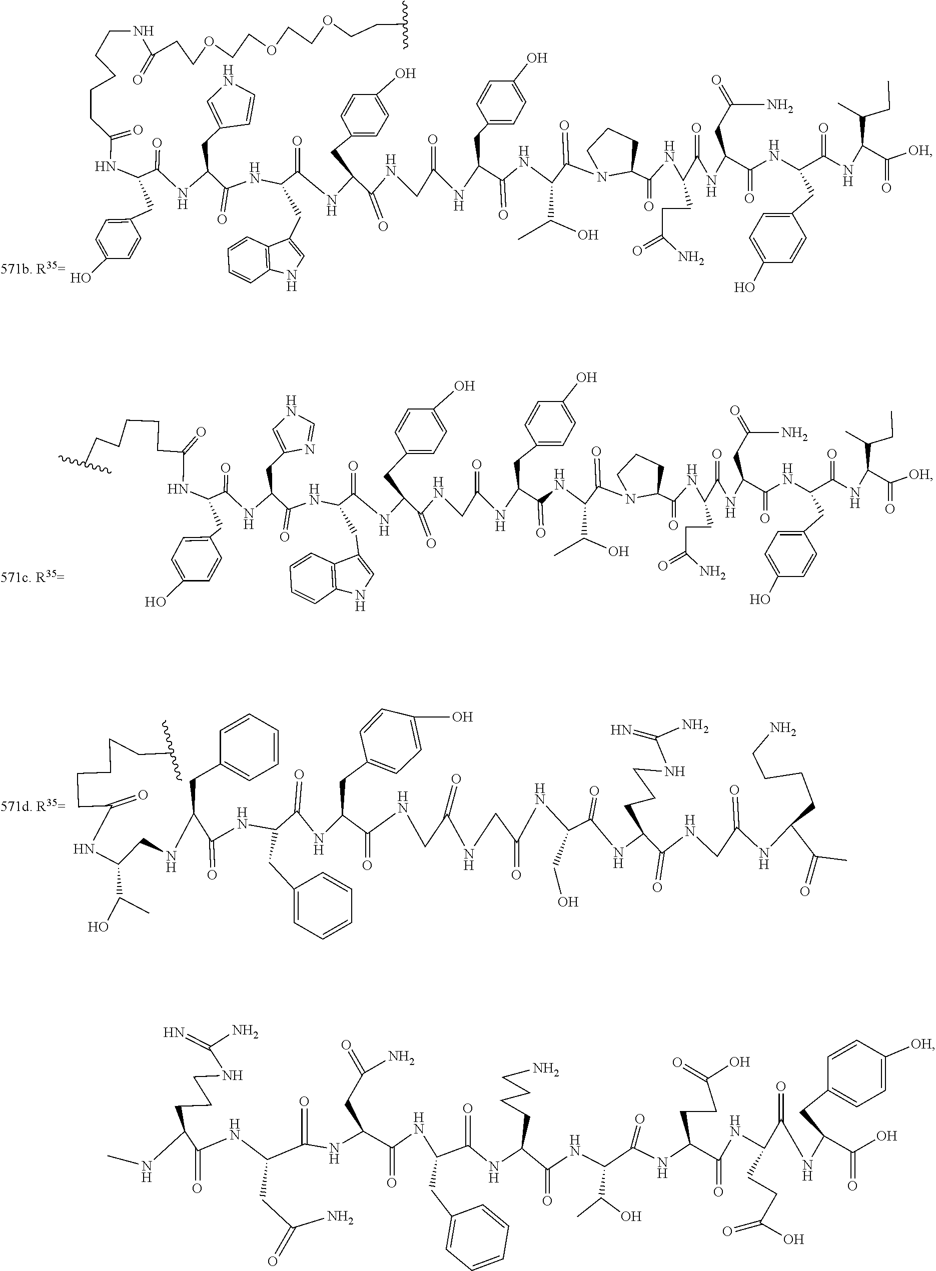

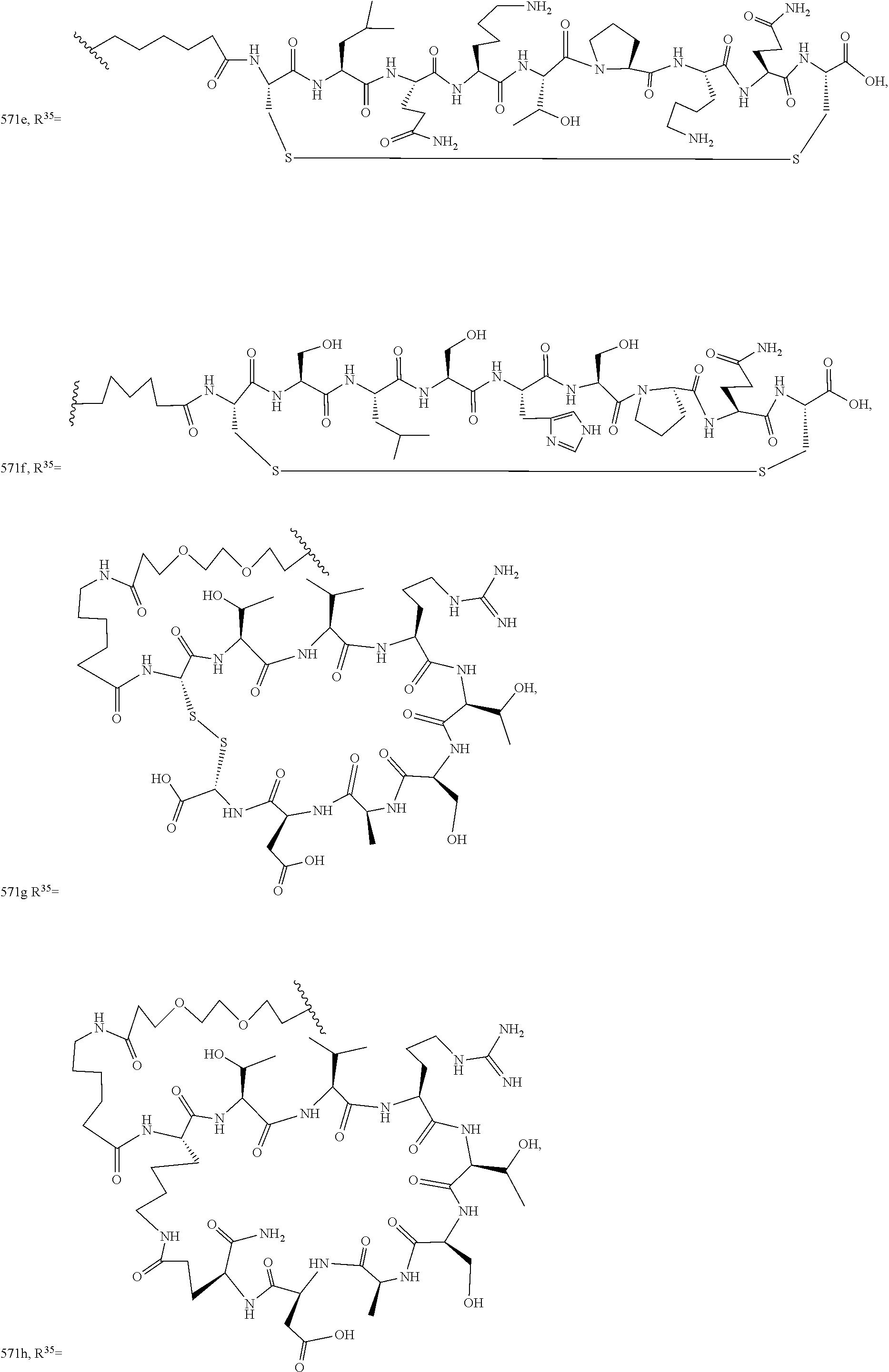

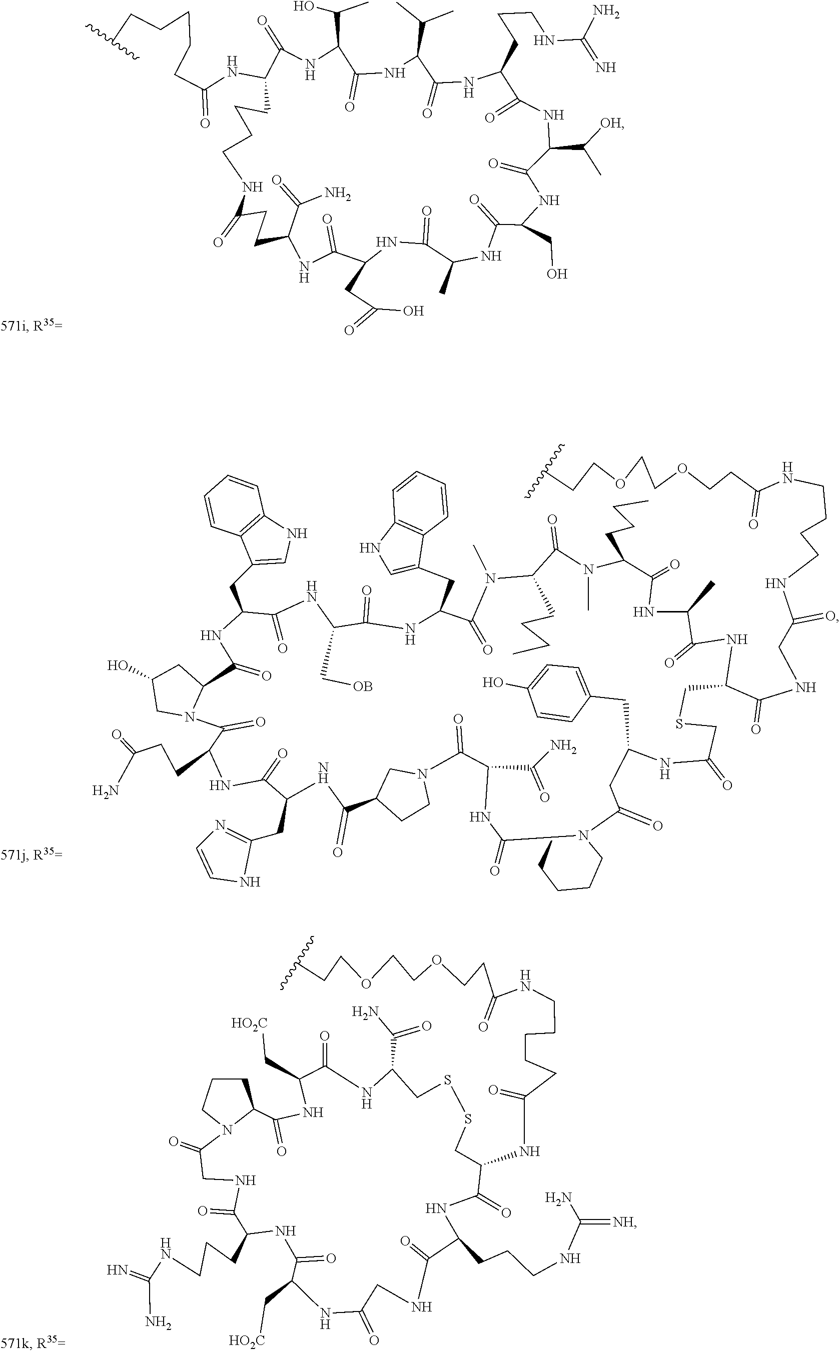

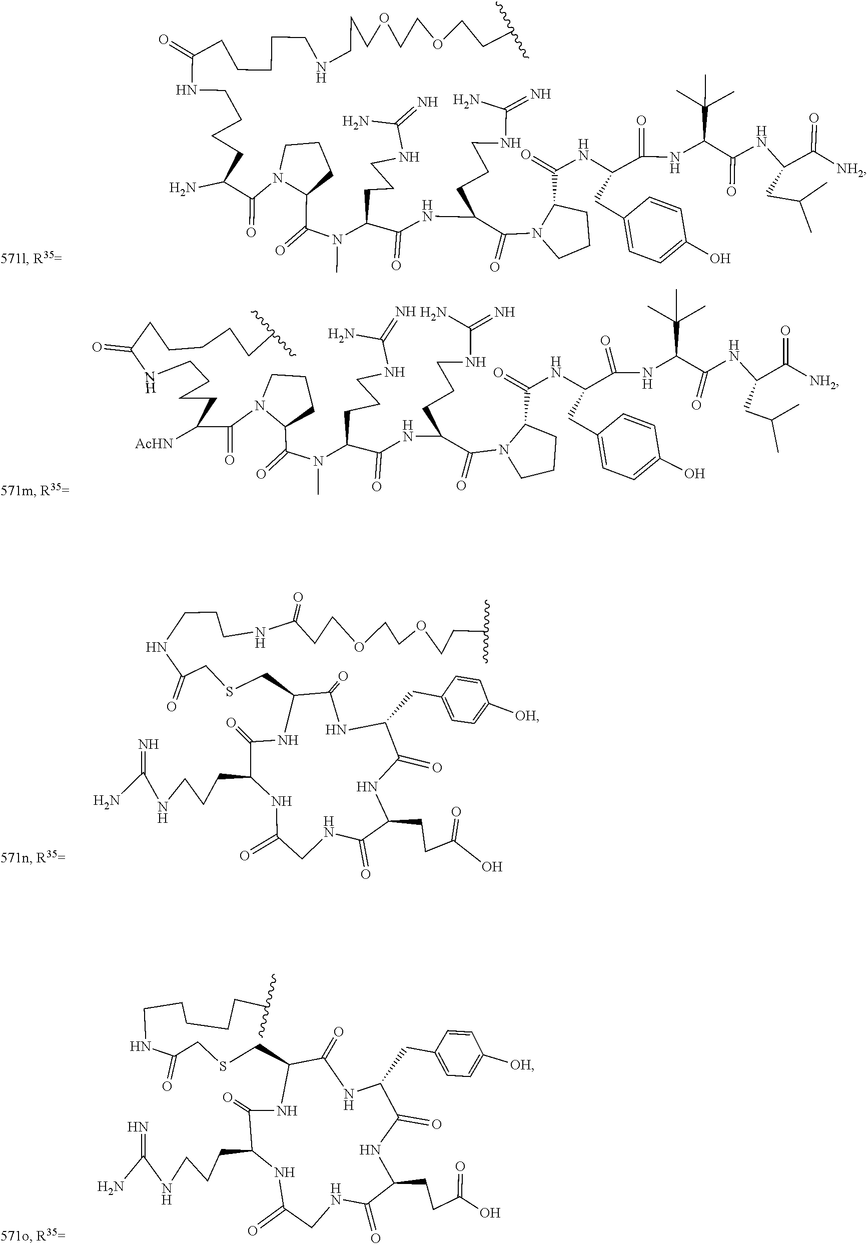

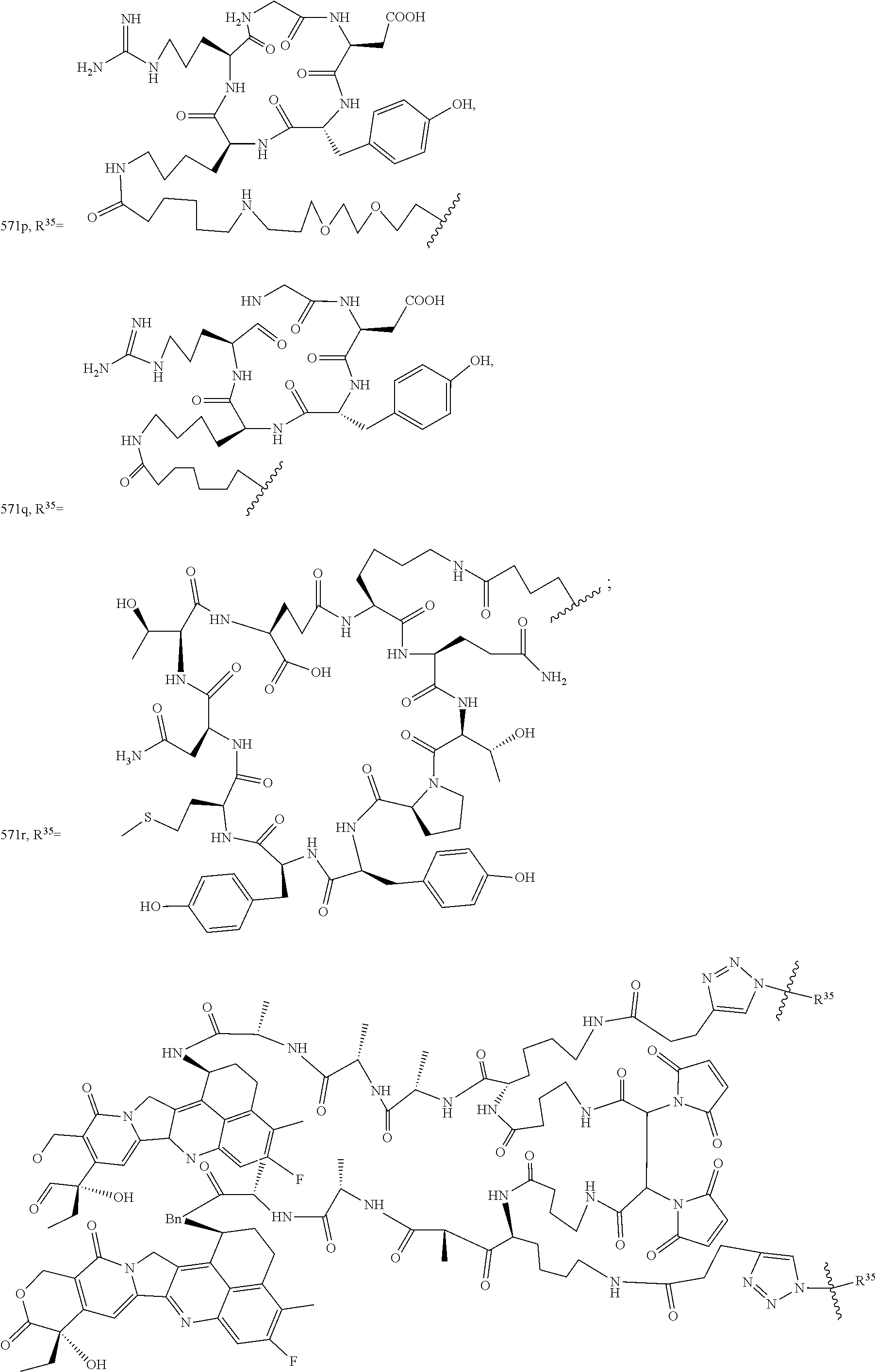

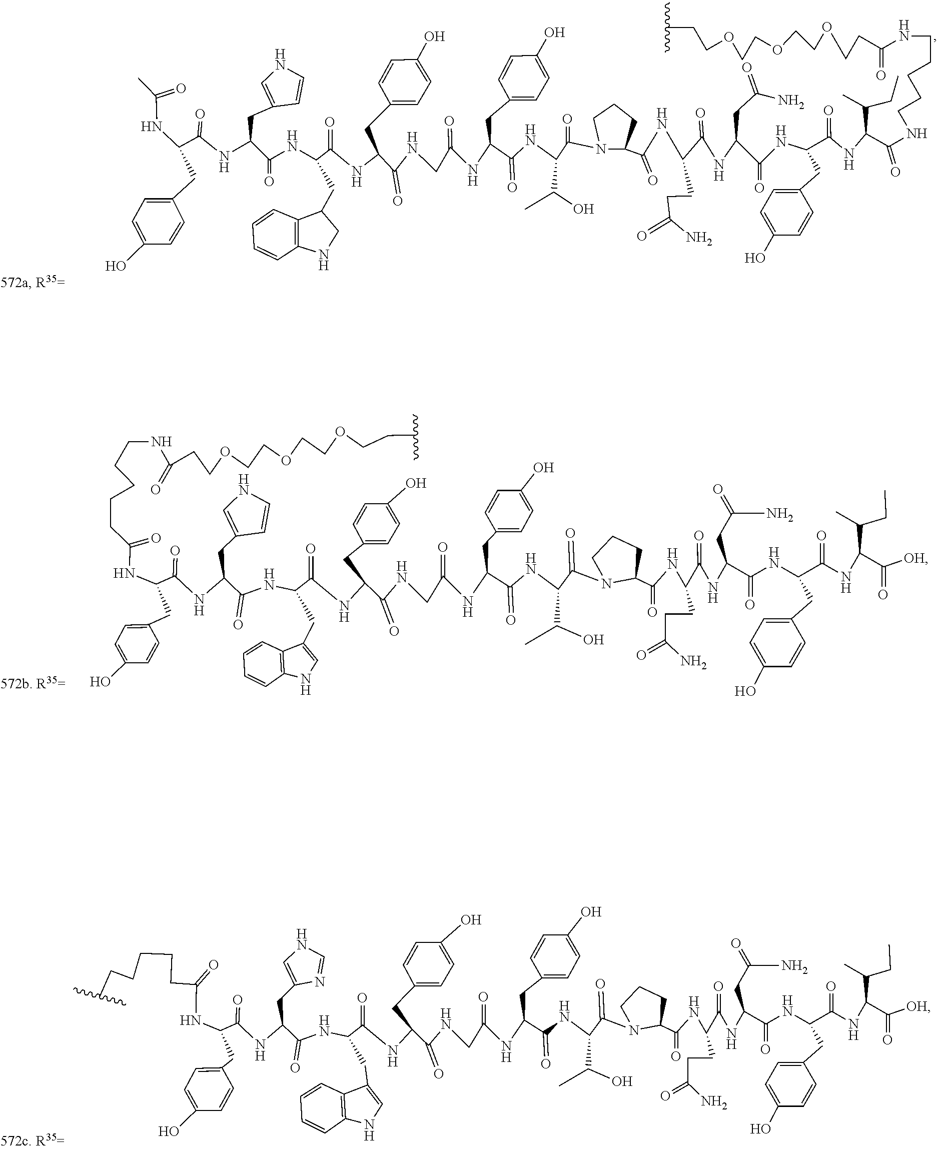

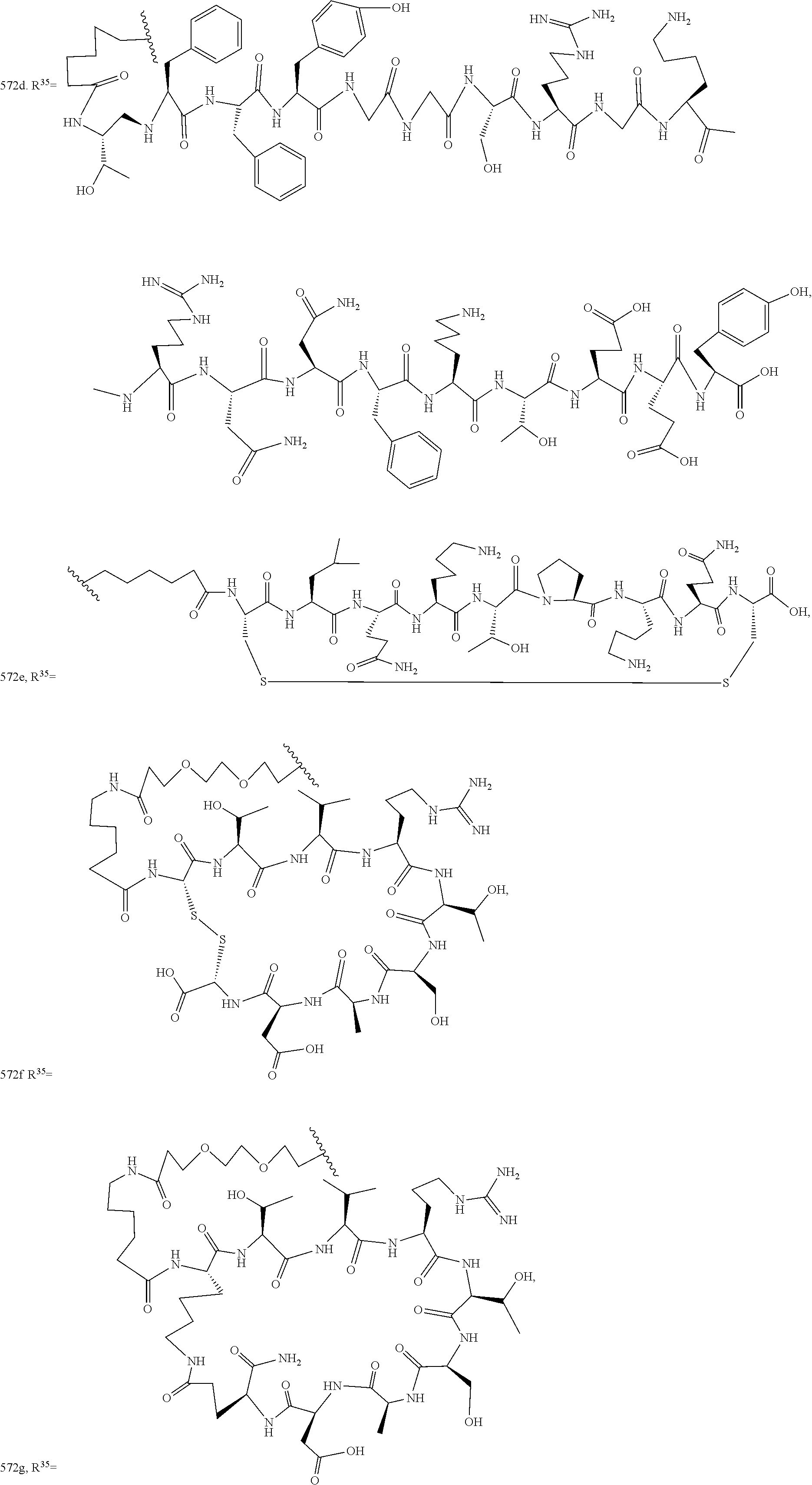

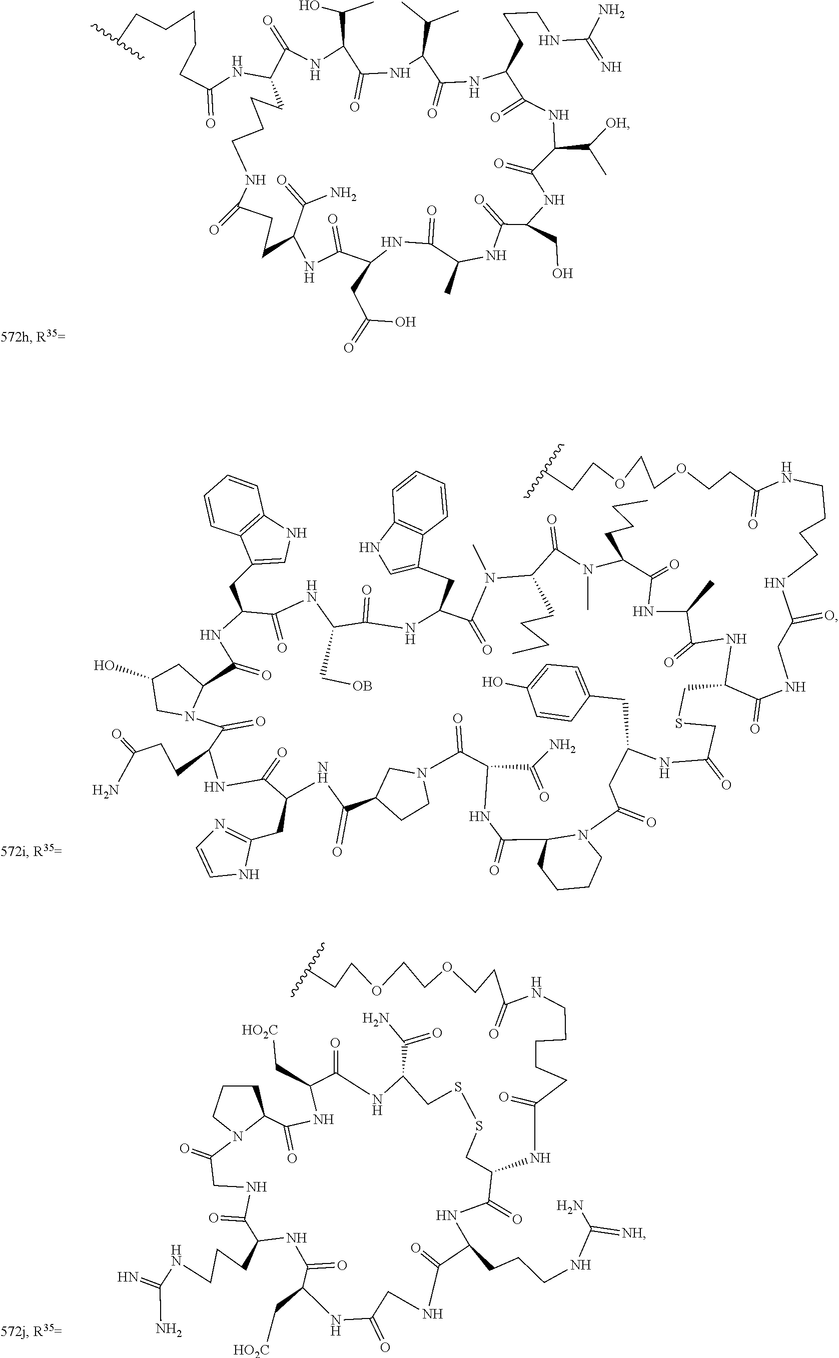

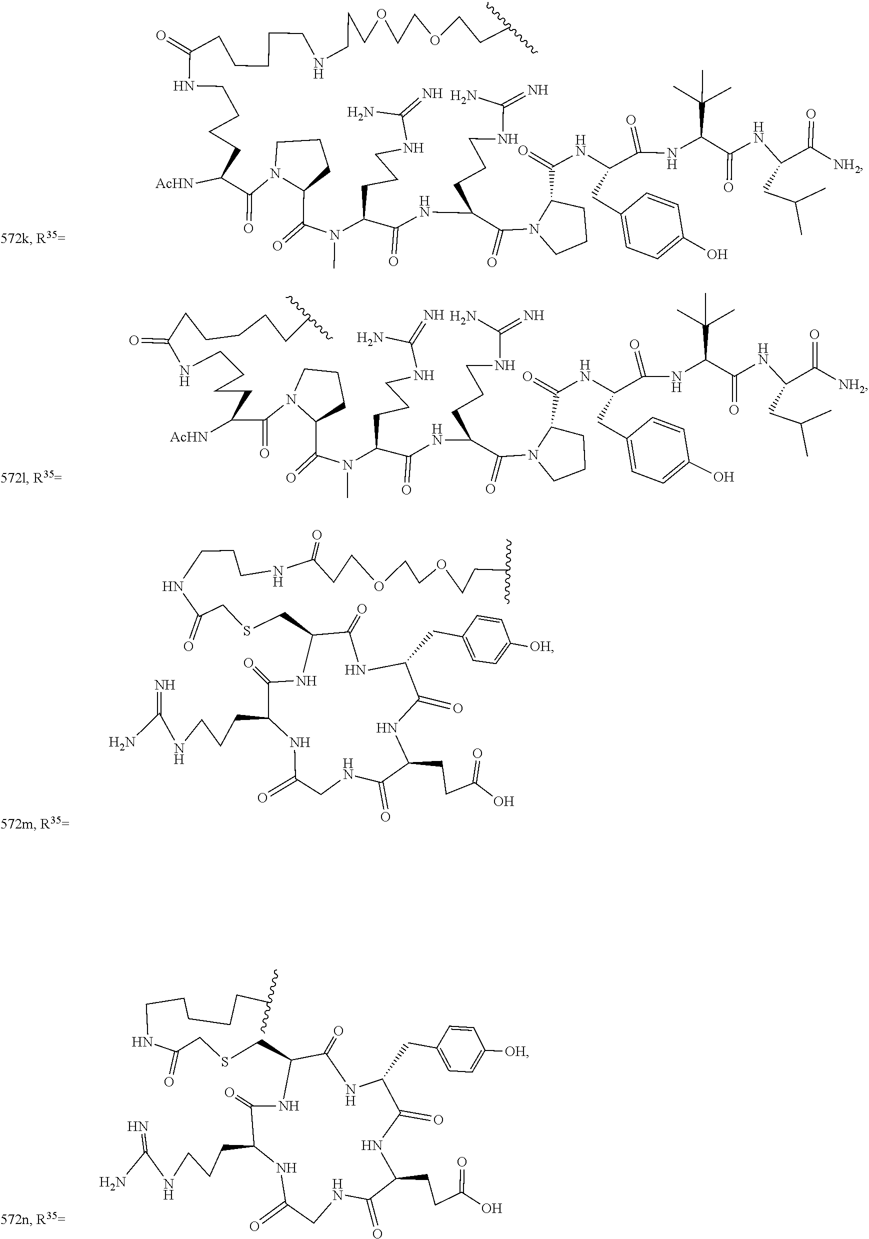

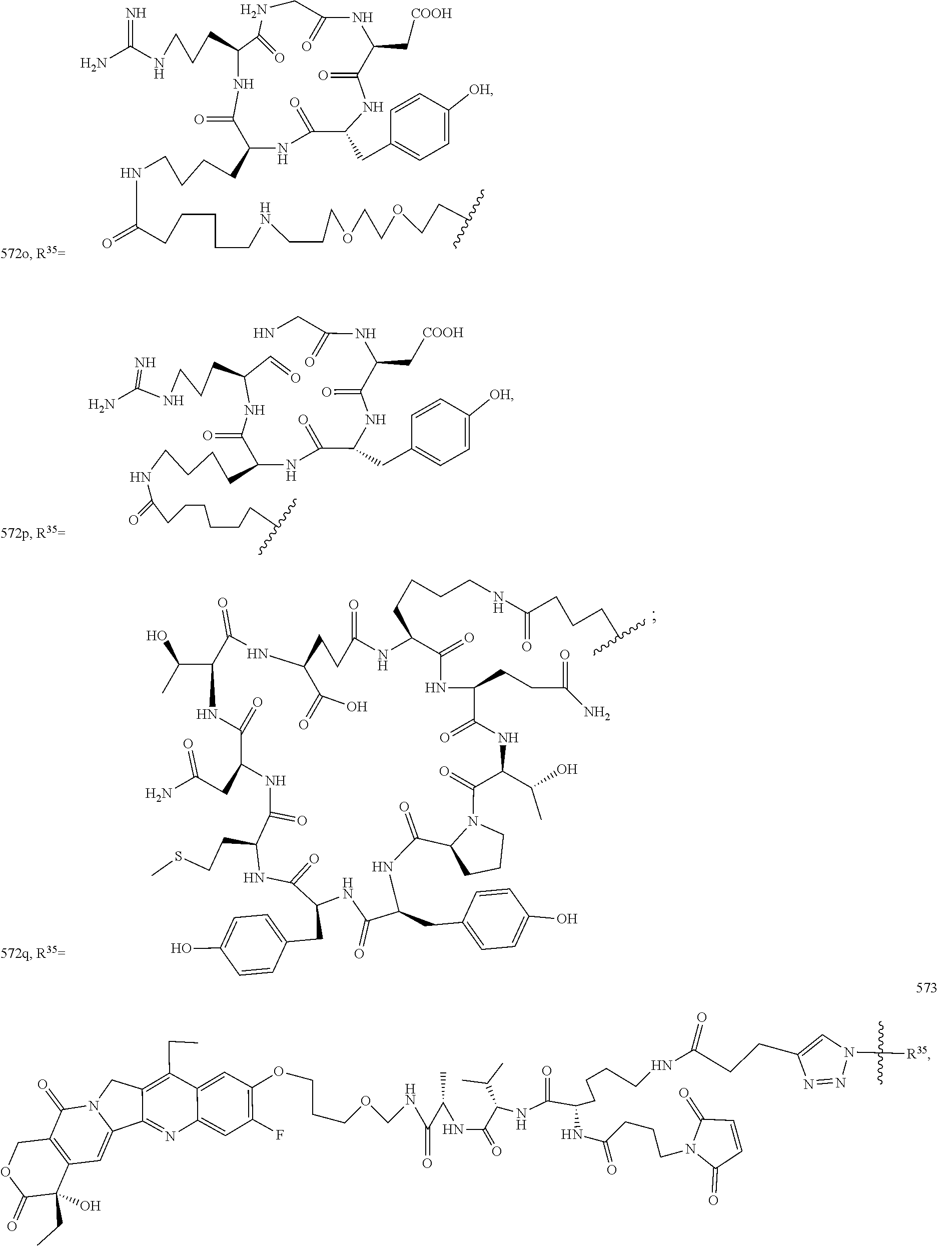

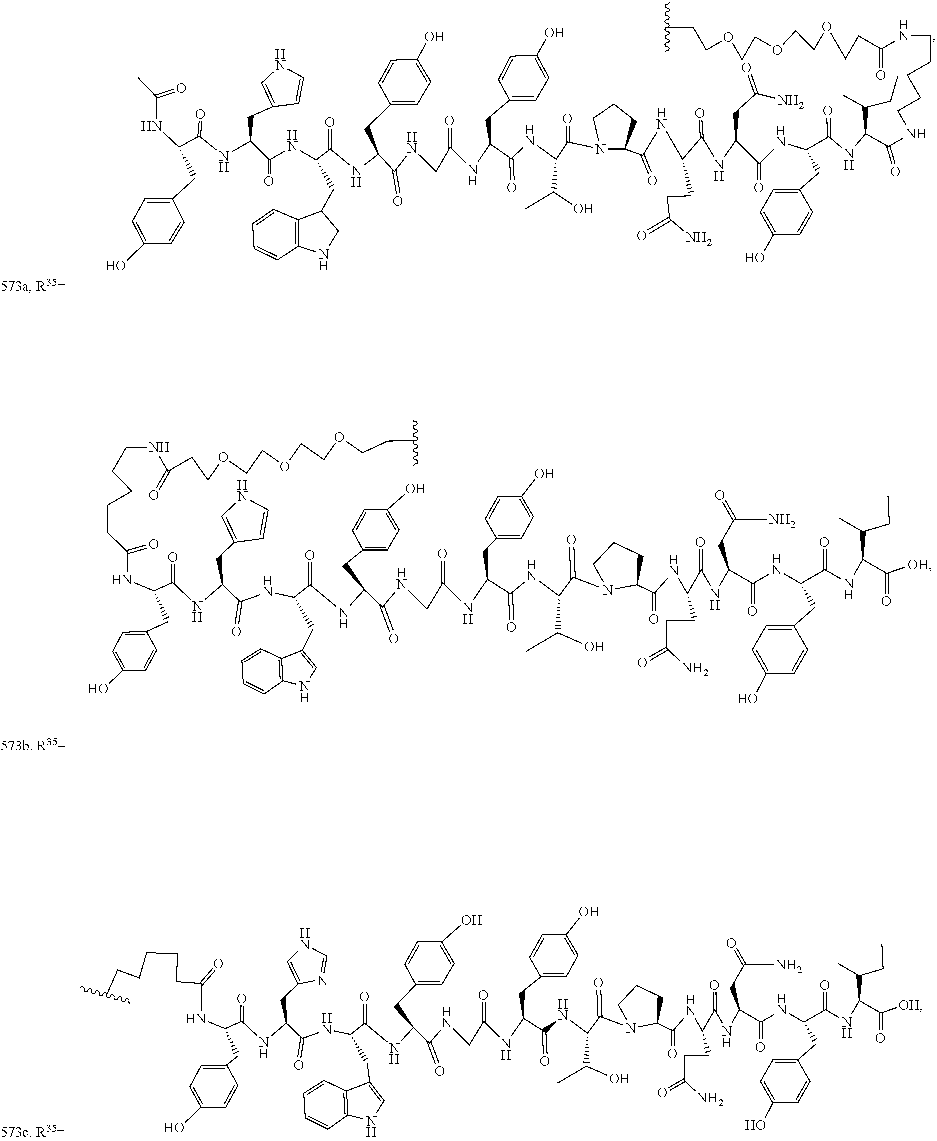

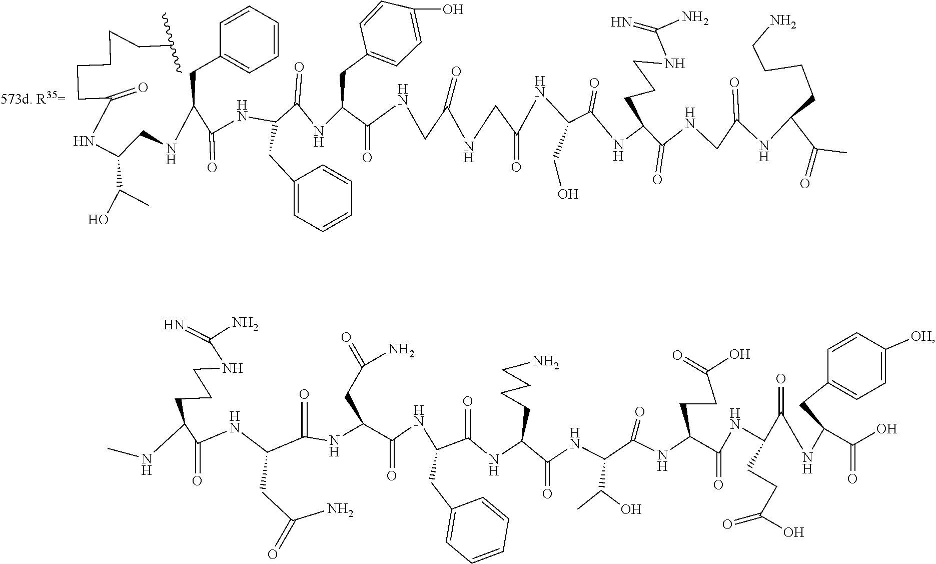

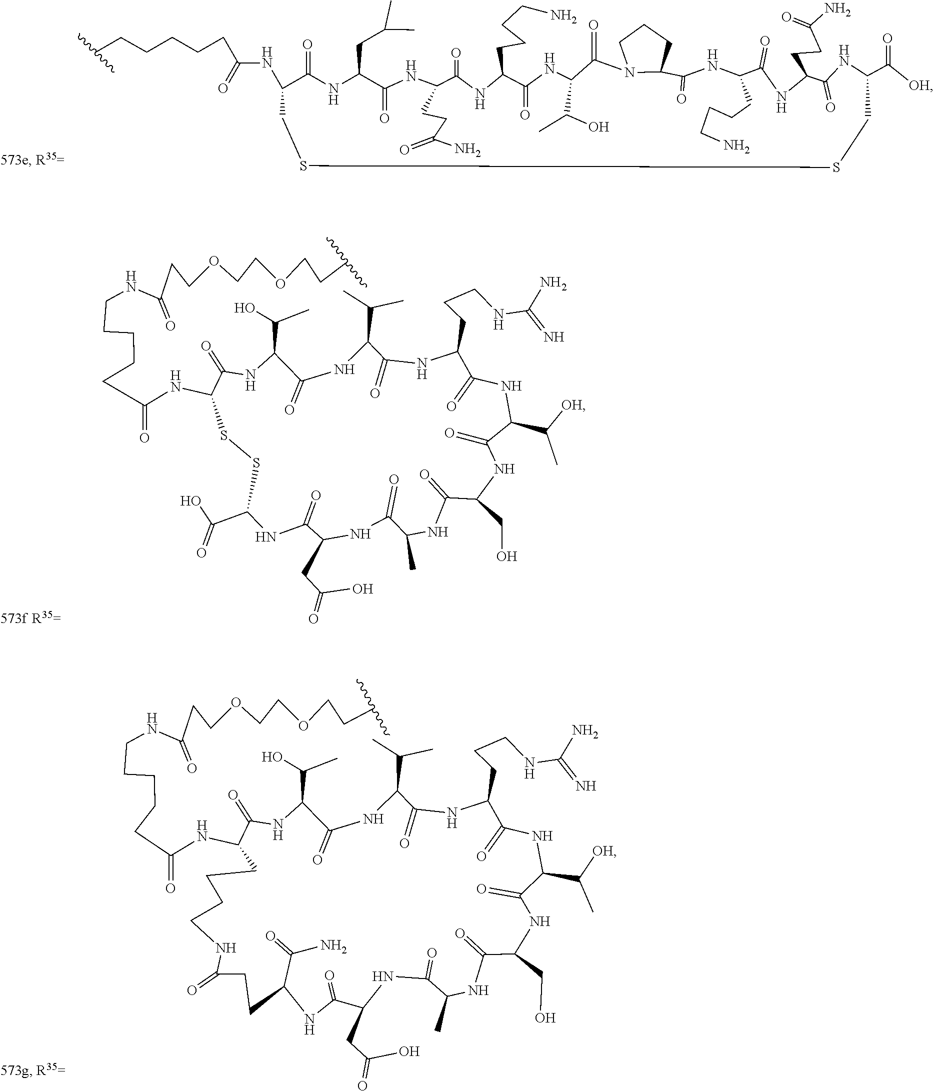

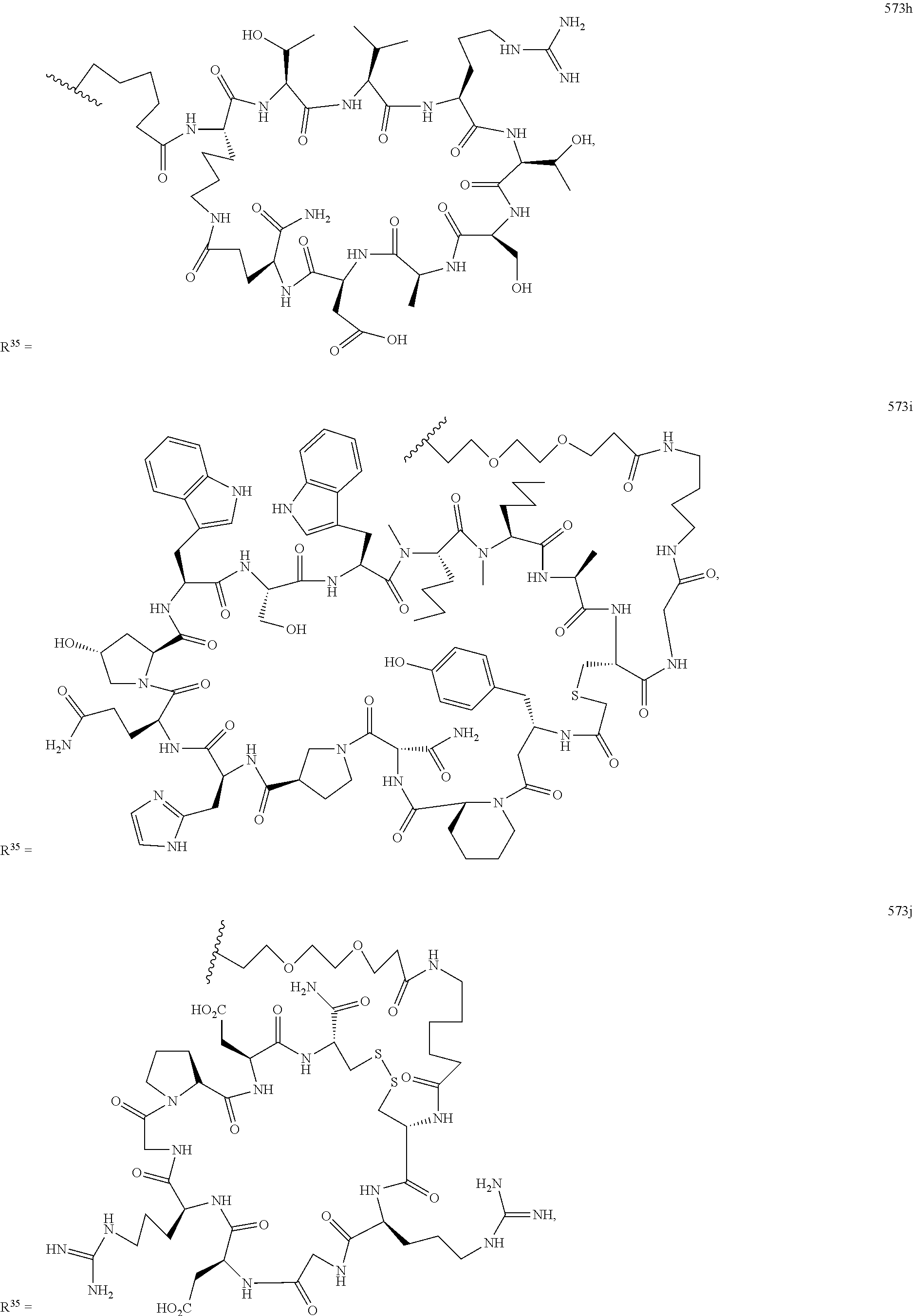

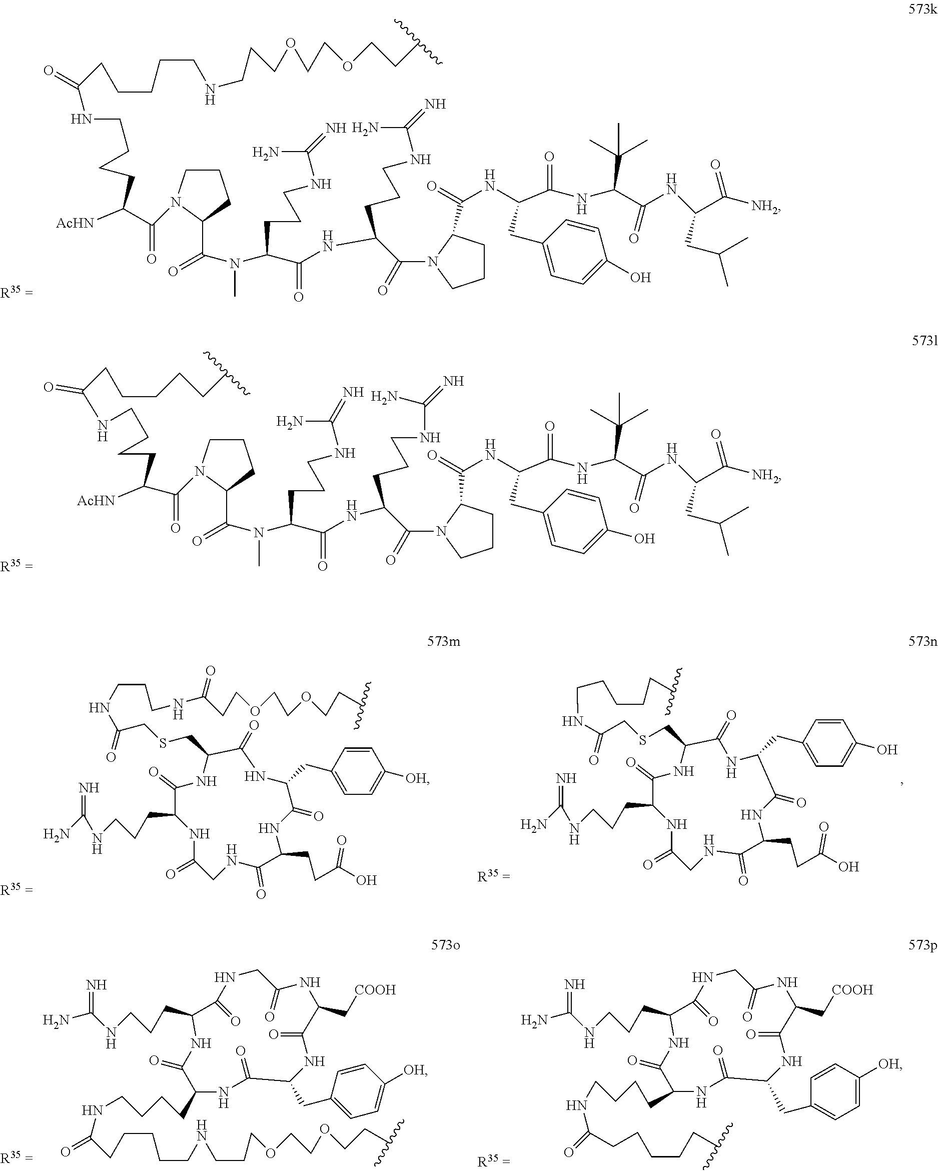

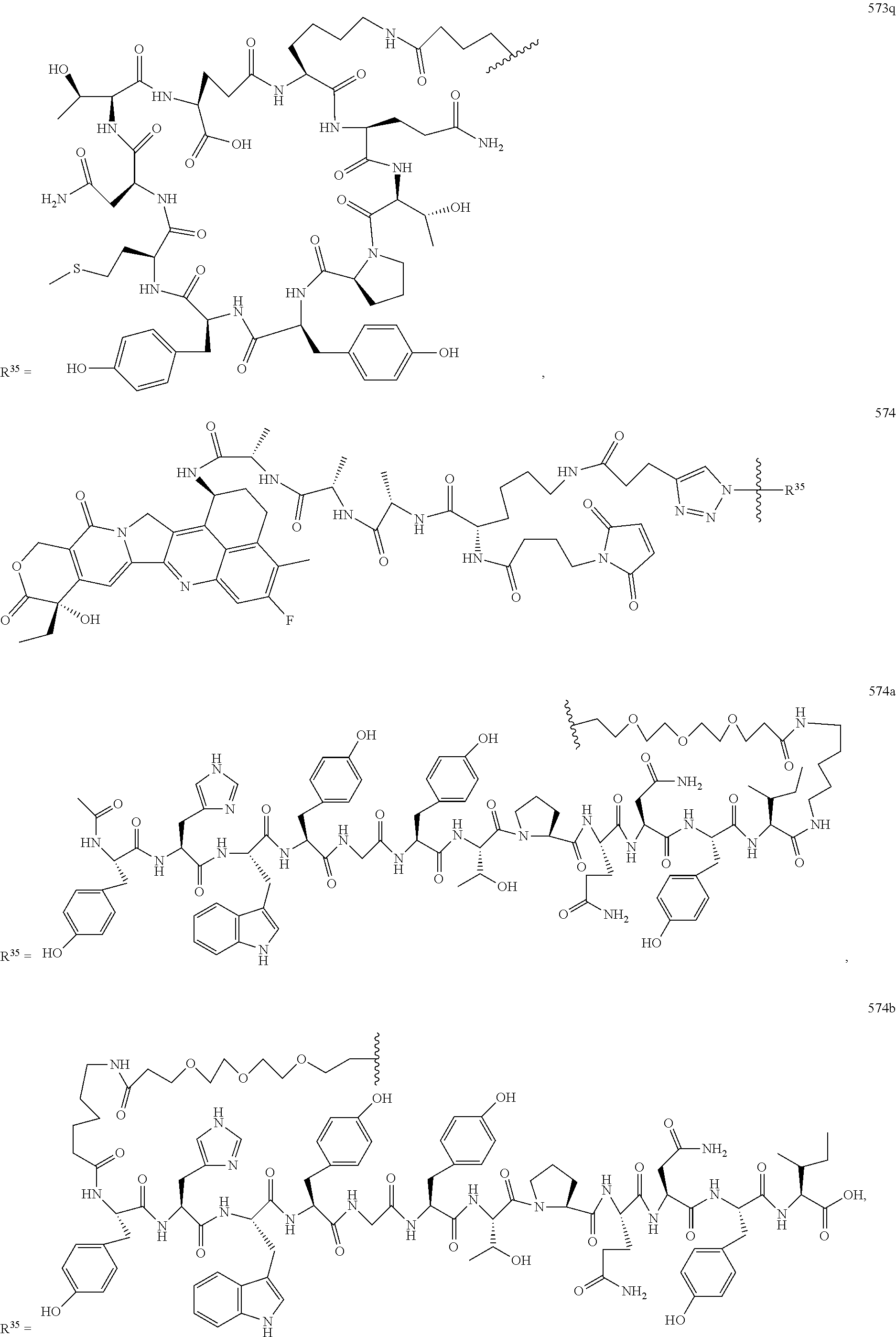

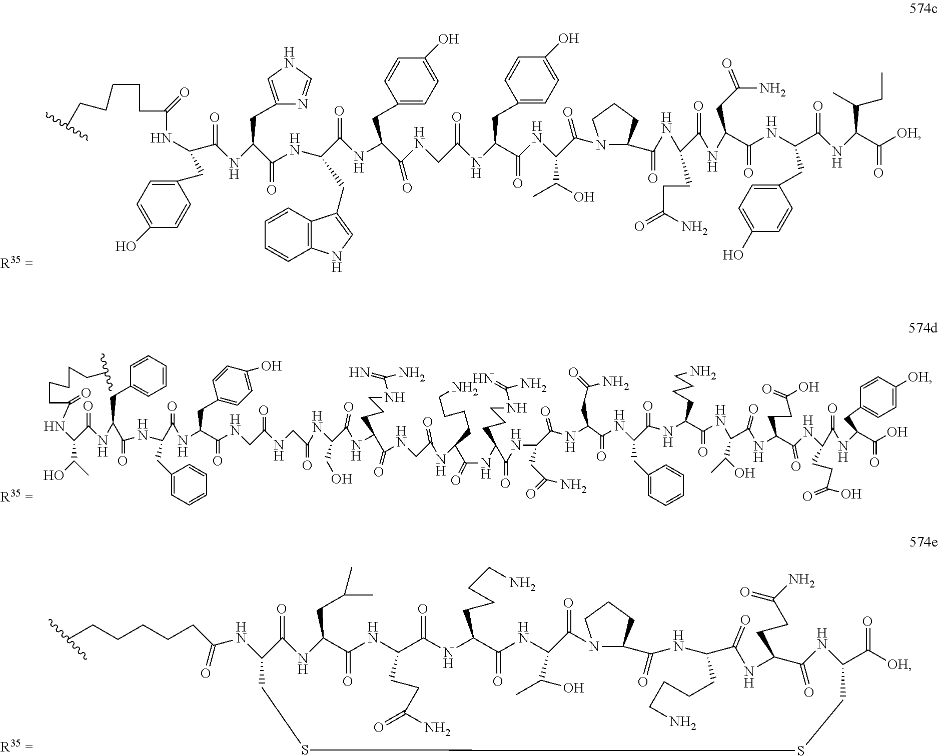

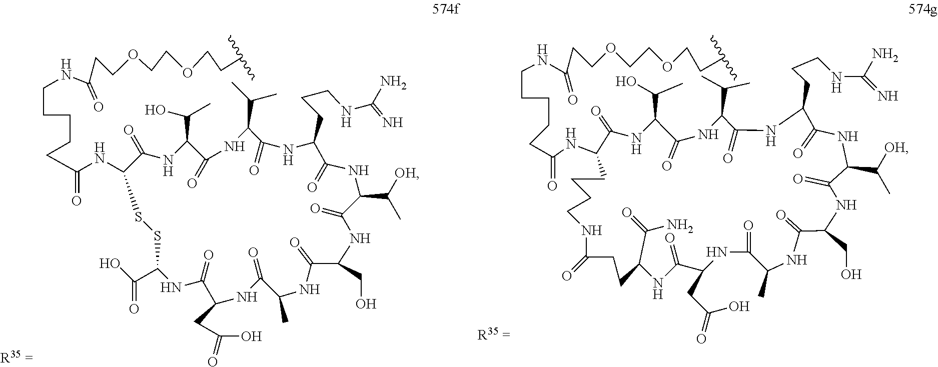 [SMILES: *CCOCCOCCC(=O)NCCCCCC(=O)N[C@H]1SS[C@@H](C(=O)O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O ; *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

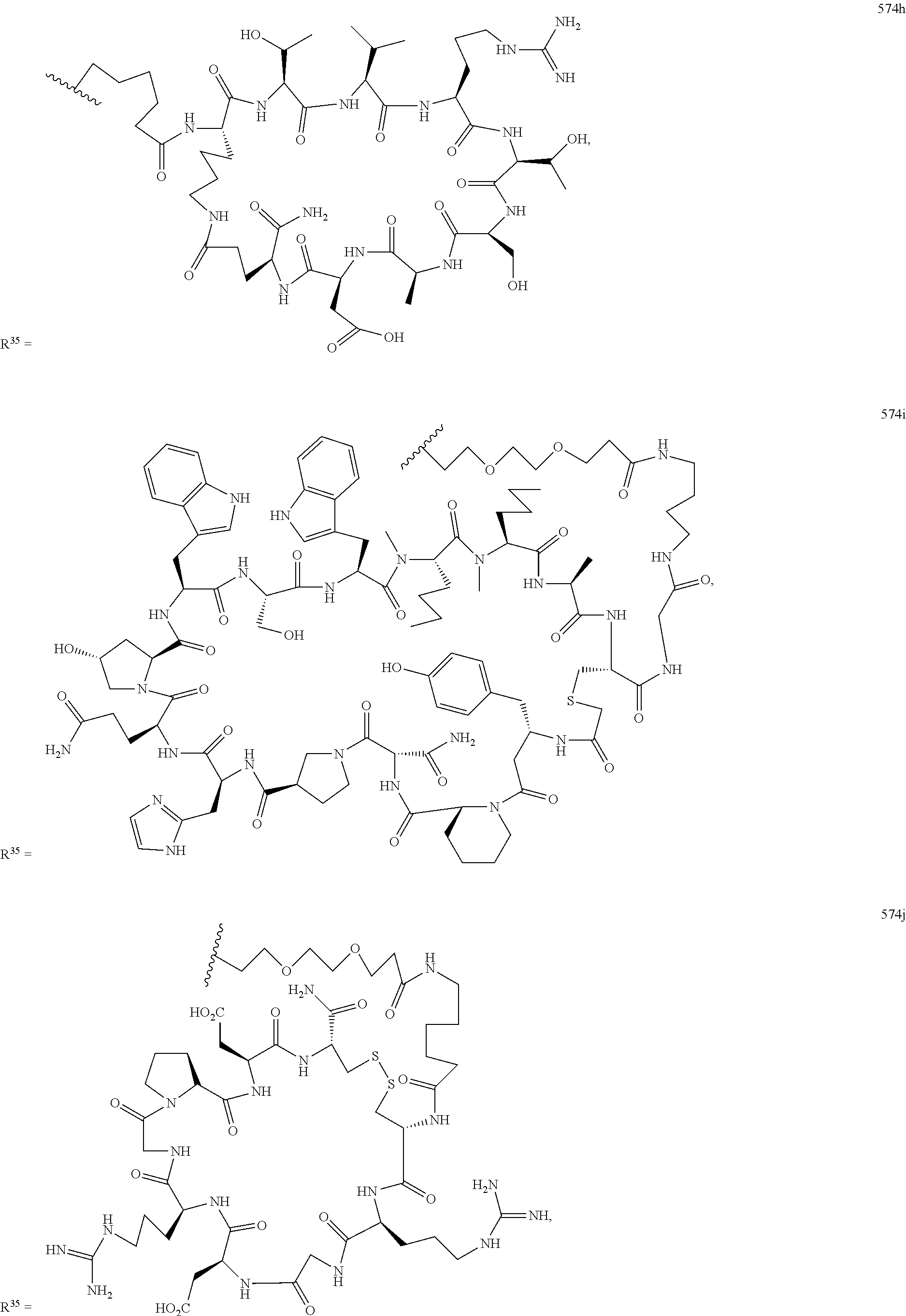

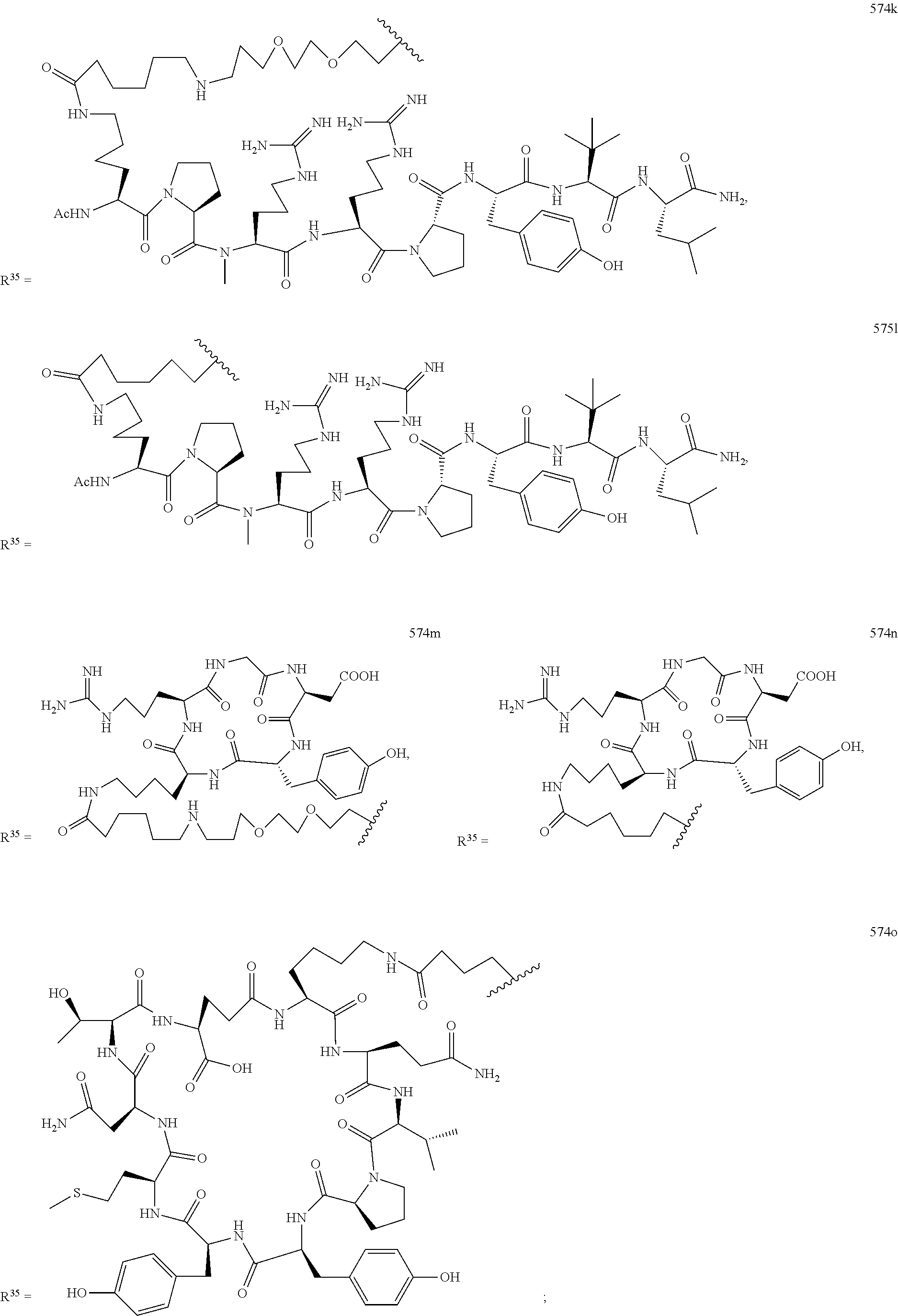

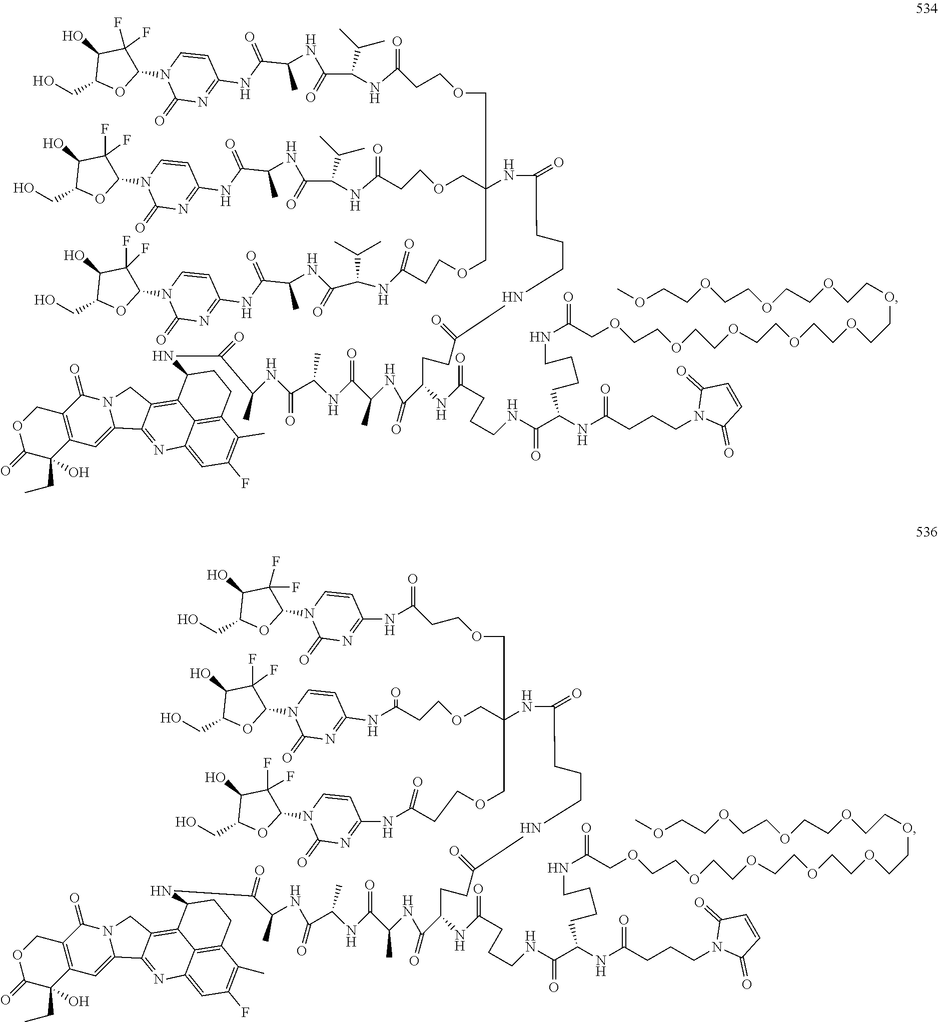

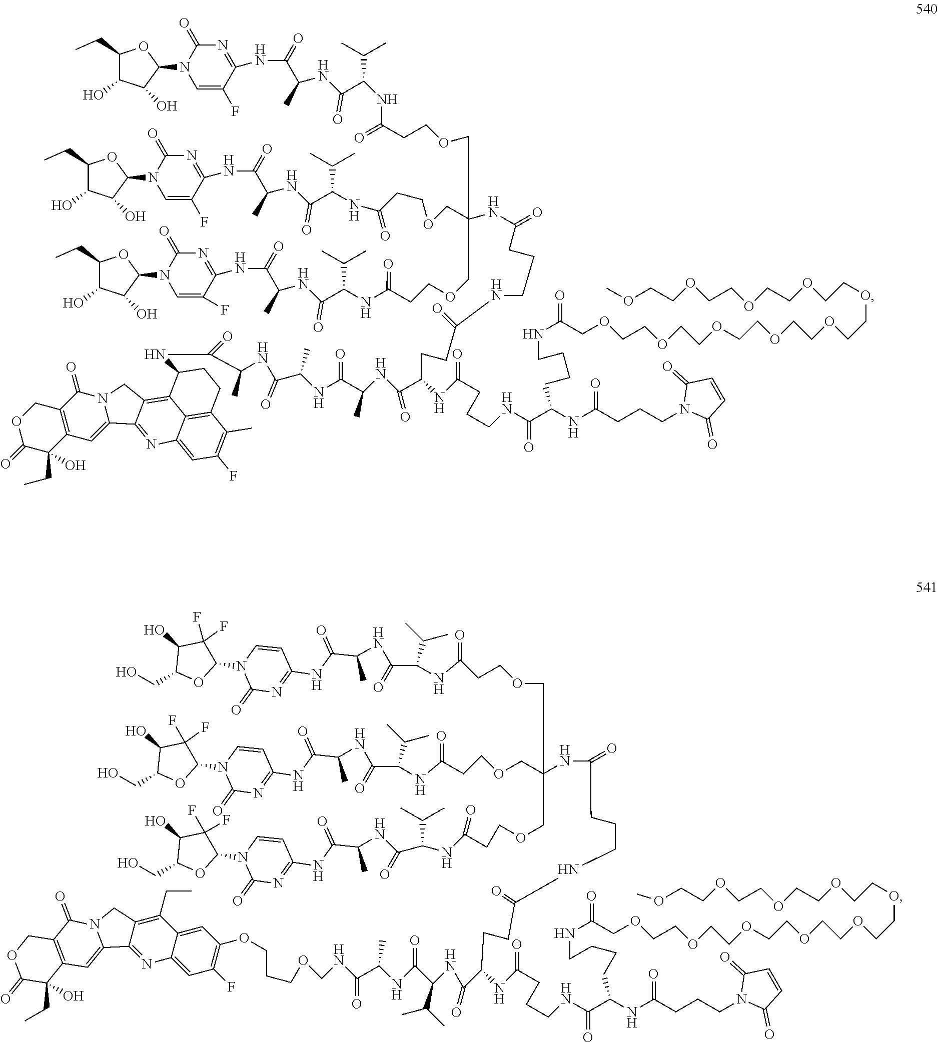

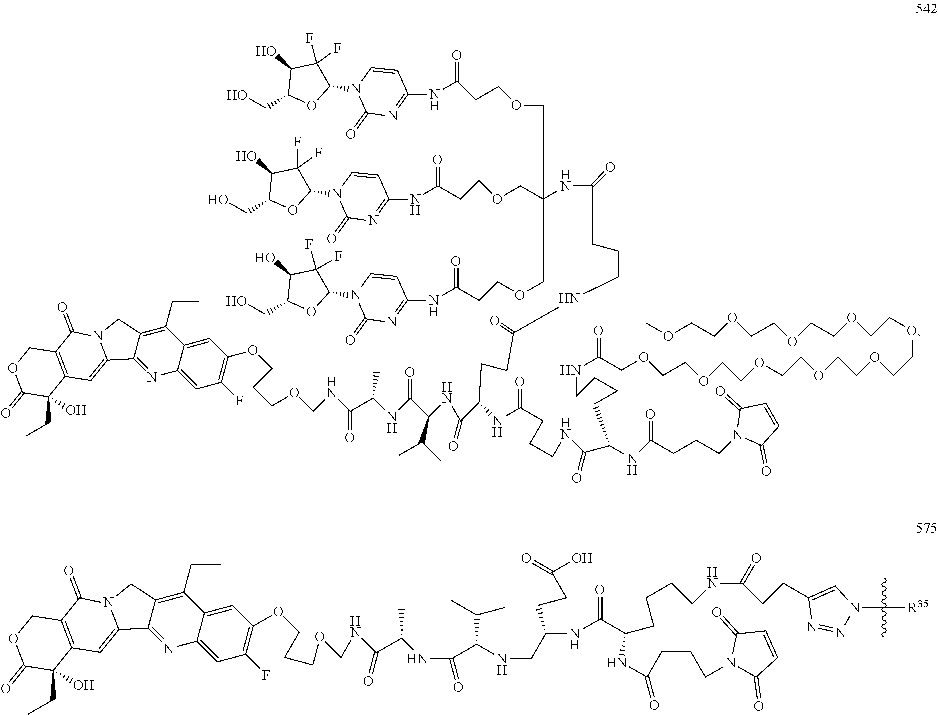

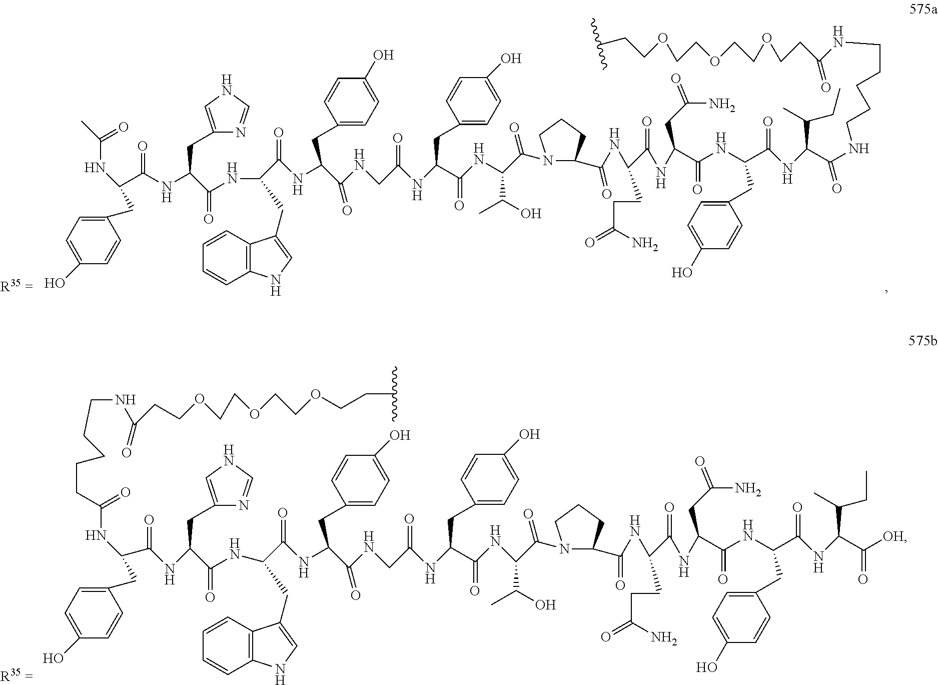

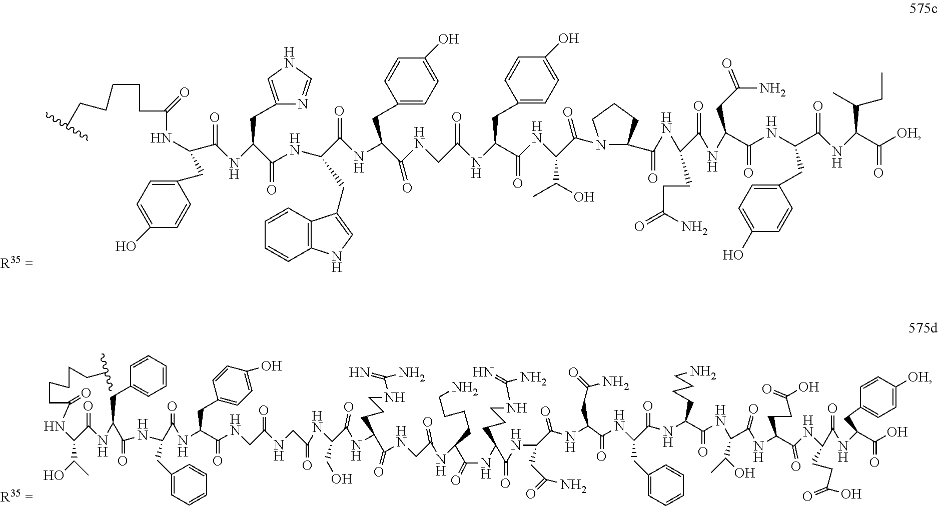

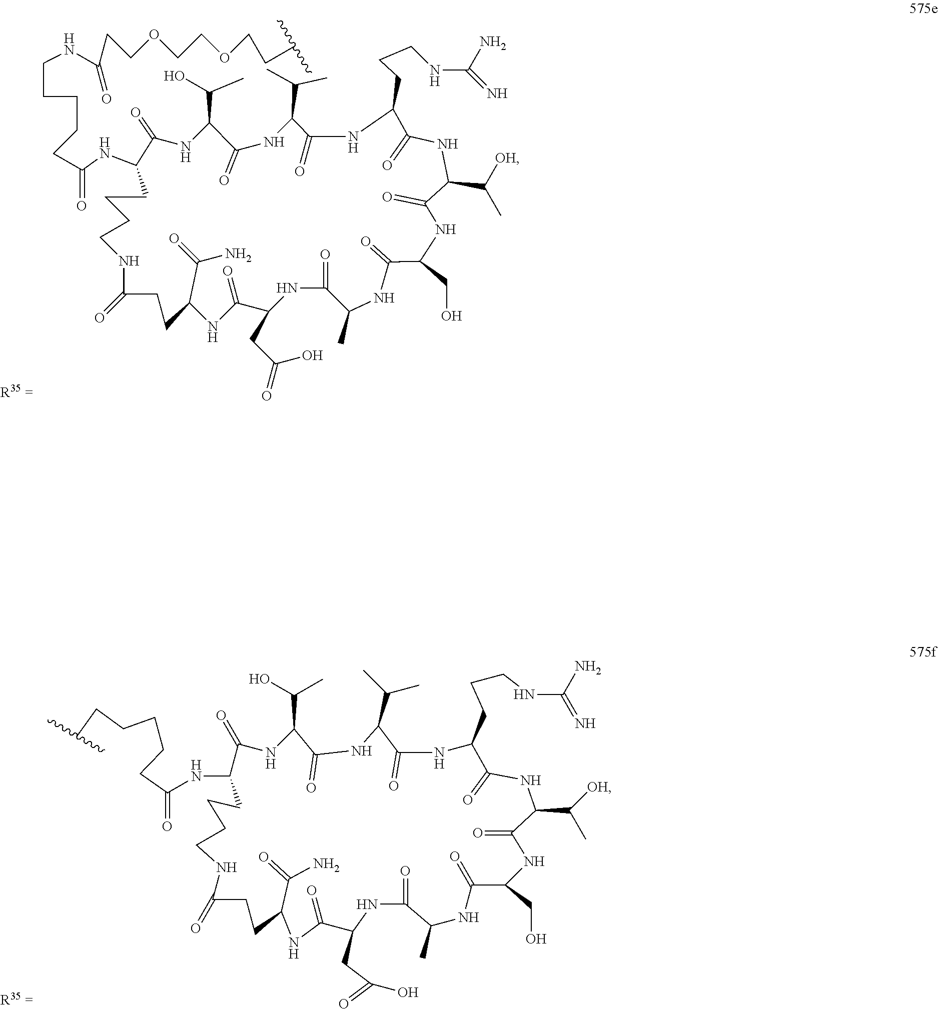 [SMILES: *CCOCCOCCC(=O)NCCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O ; *CCCCCC(=O)N[C@H]1CCCCNC(=O)CC[C@@H](C(N)=O)NC(=O)[C@H](CC(=O)O)NC(=O)[C@H](C)NC(=O)[C@H](CO)NC(=O)[C@H](C(C)O)NC(=O)[C@H](CCCNC(=N)N)NC(=O)[C@H](C(C)C)NC(=O)[C@H](C(C)O)NC1=O]

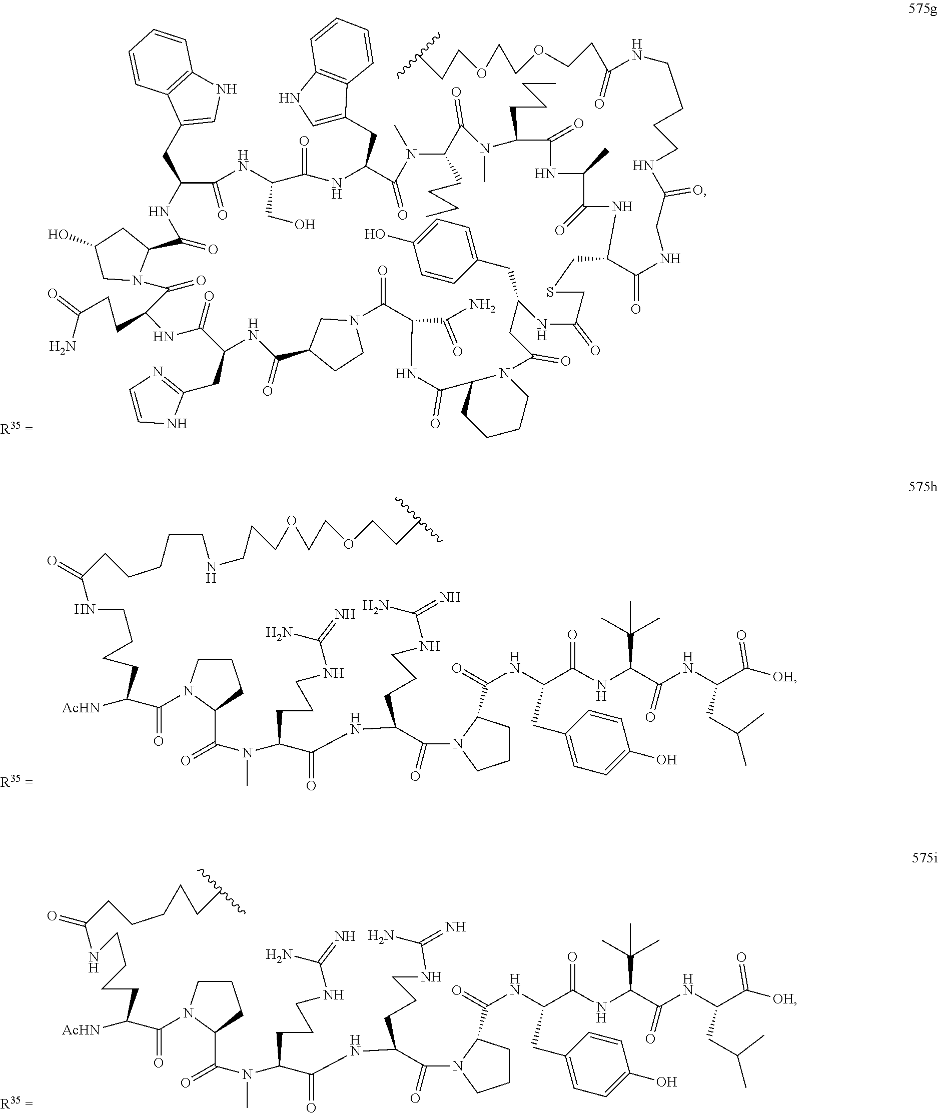

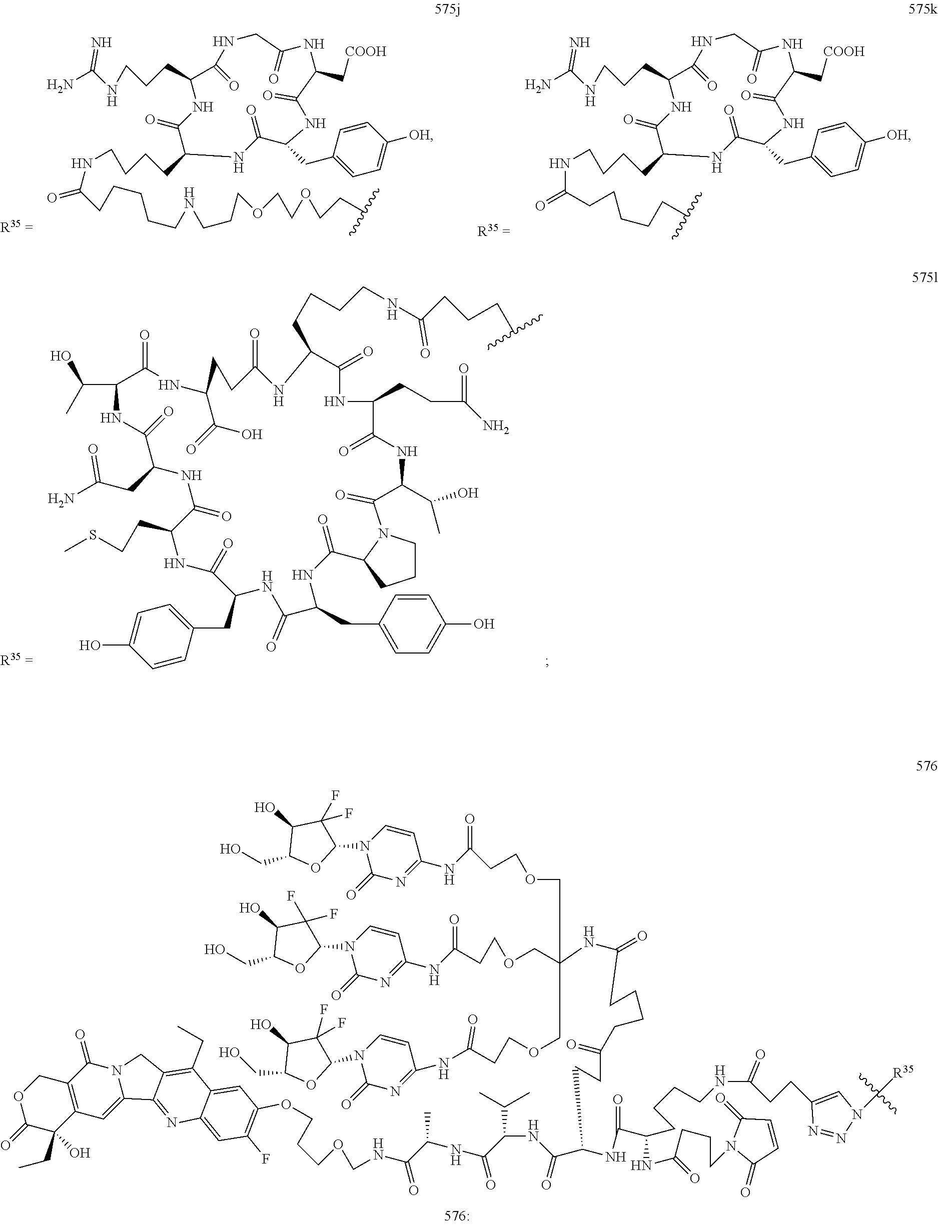

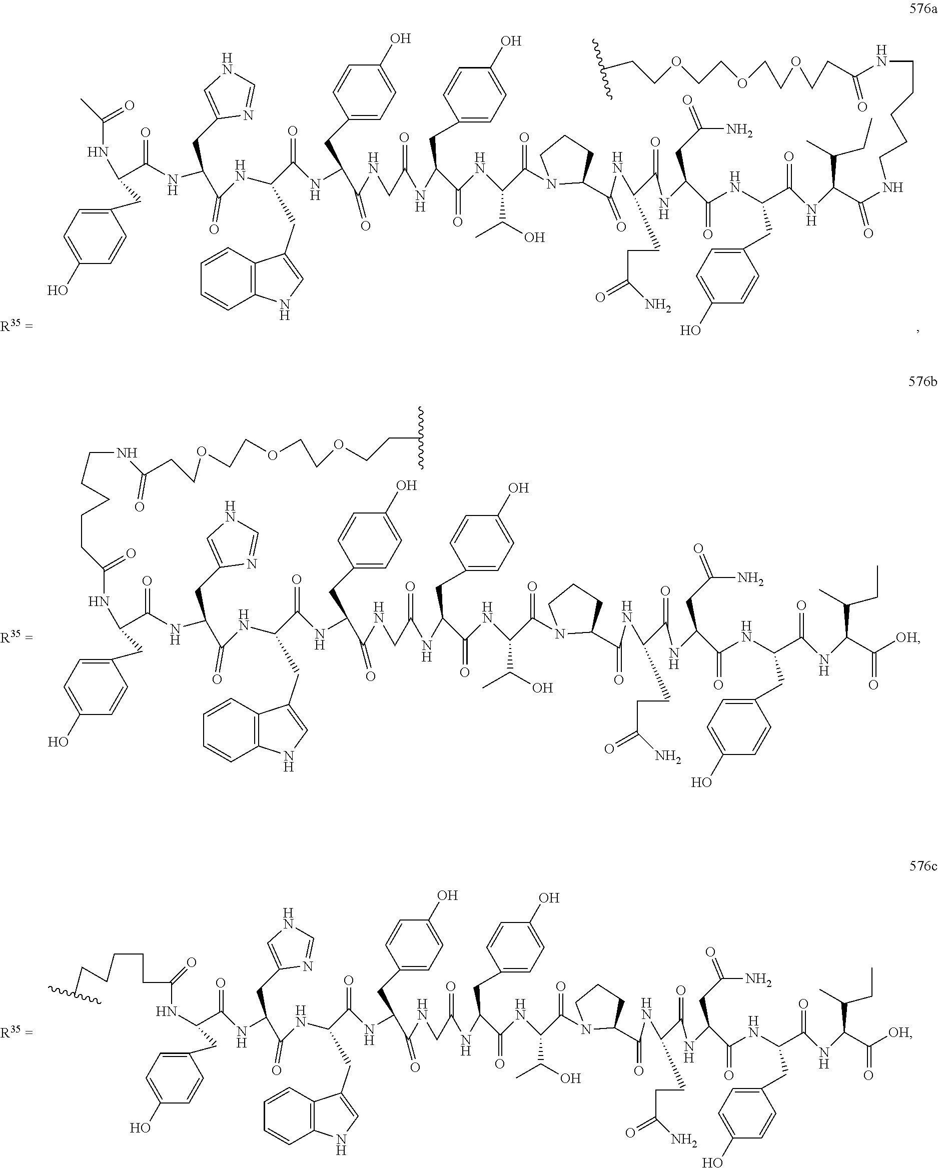

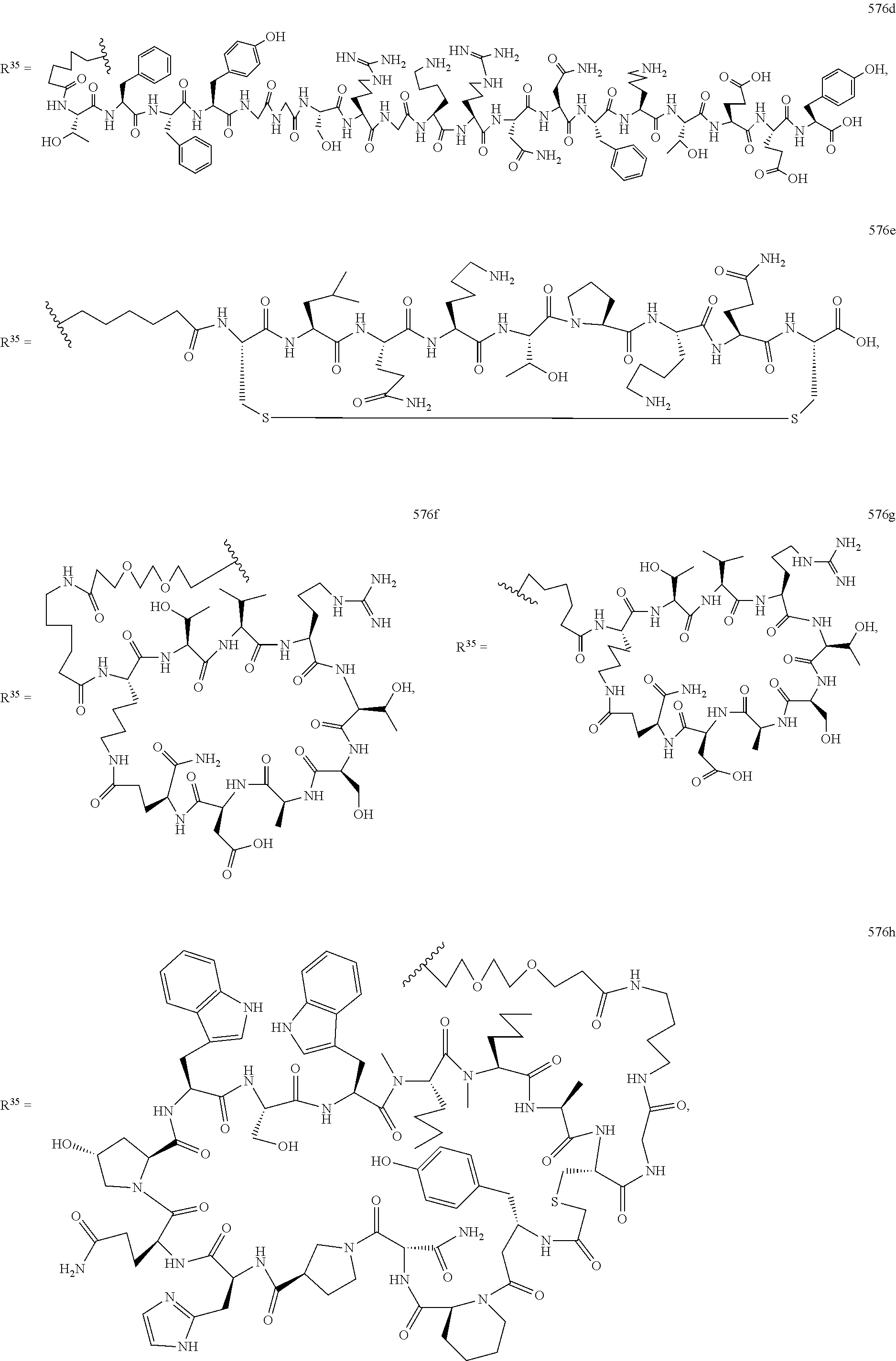

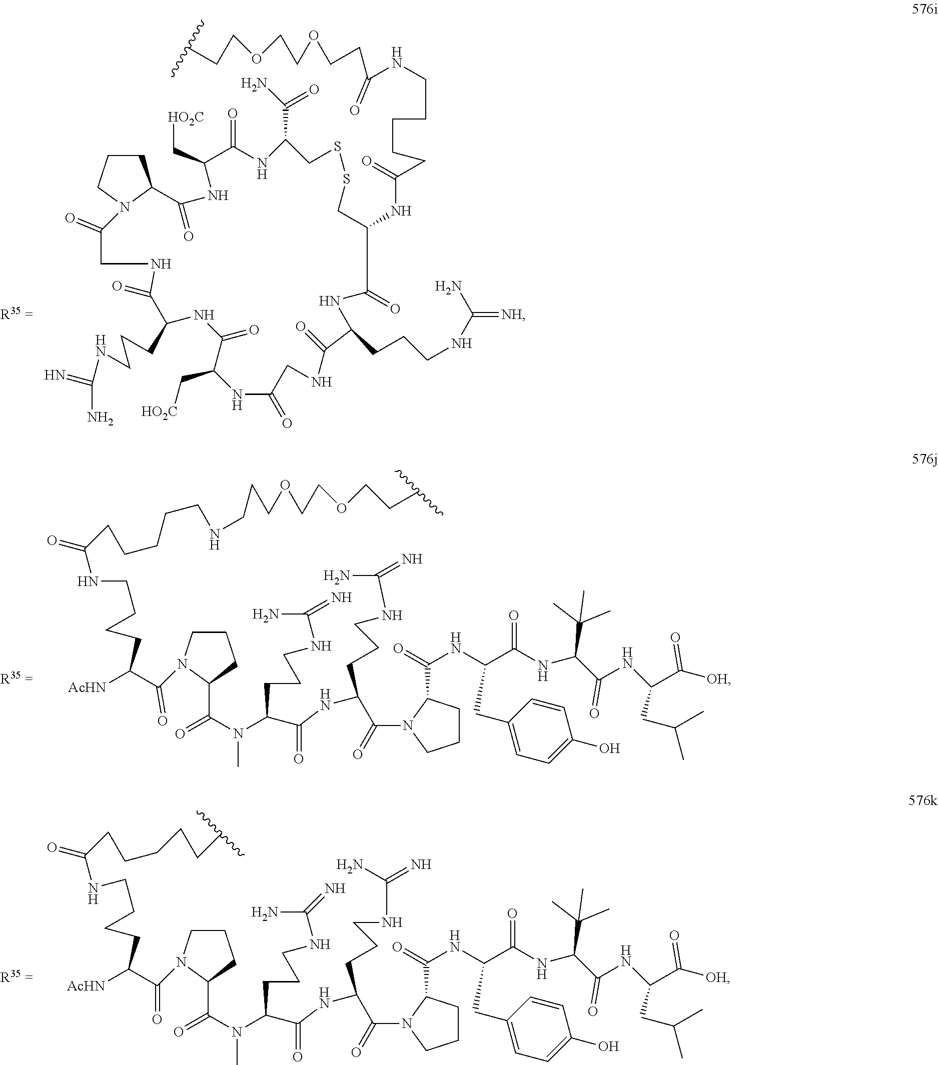

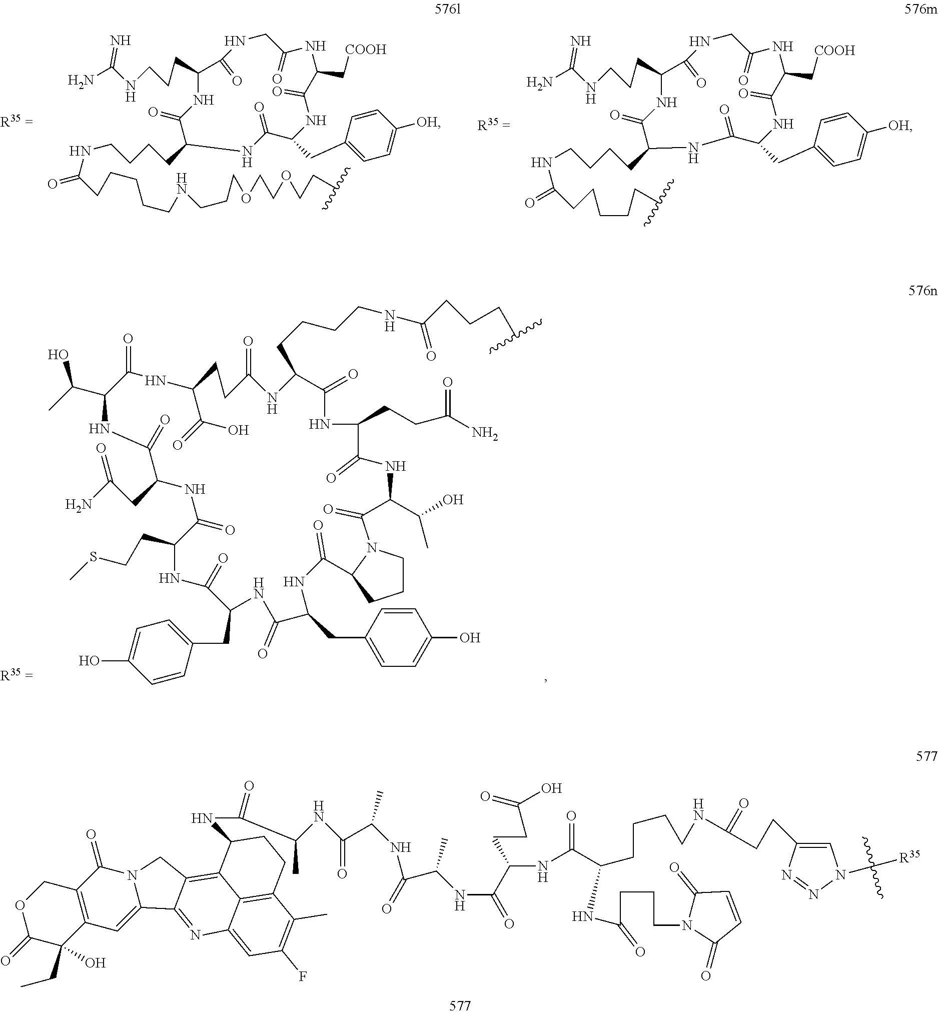

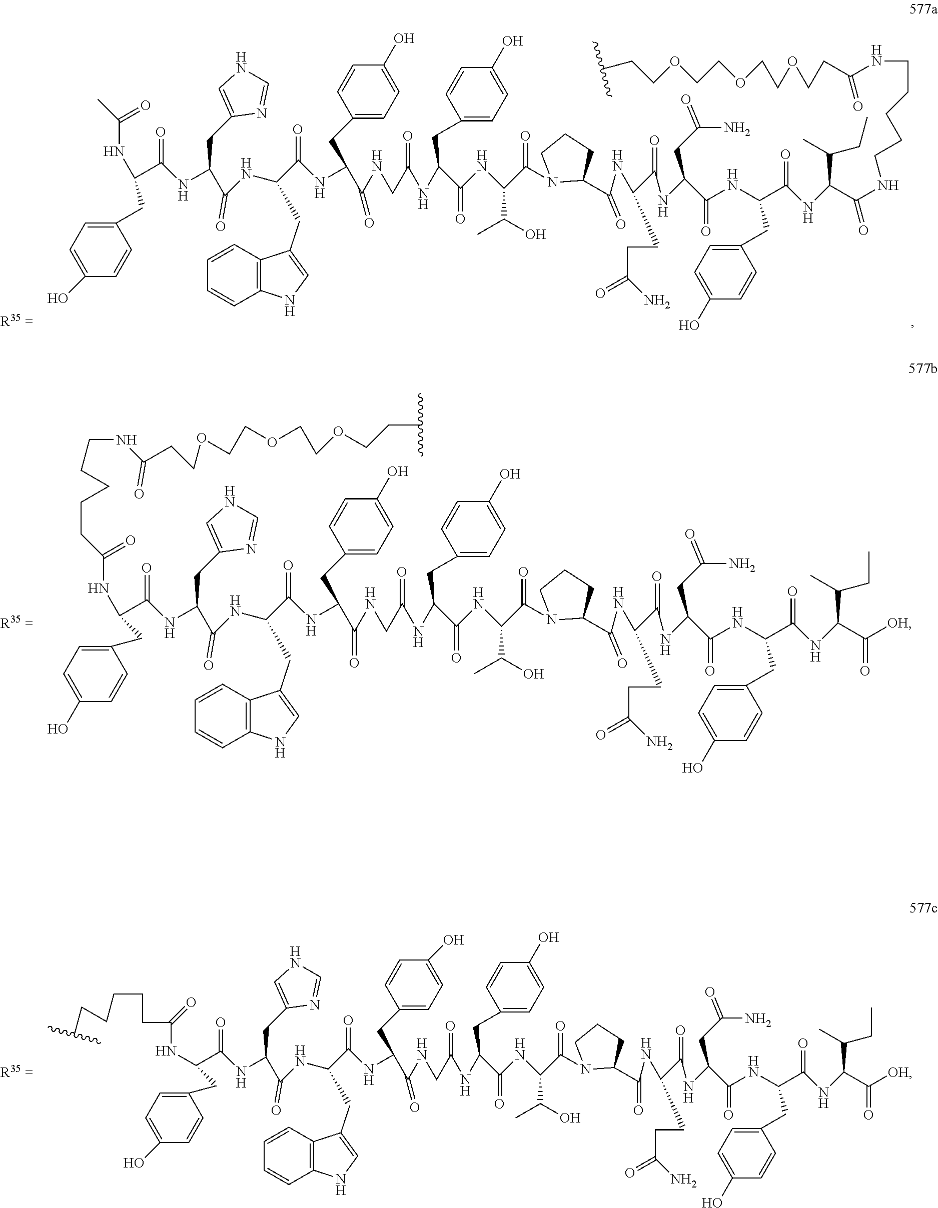

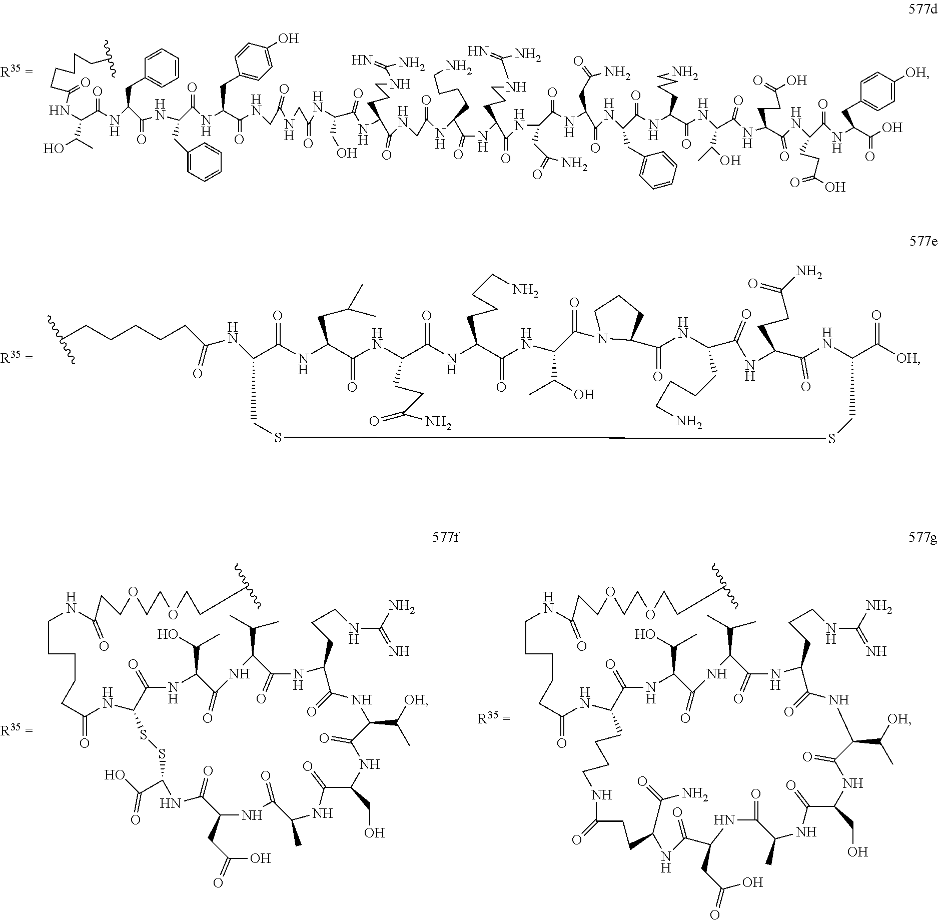

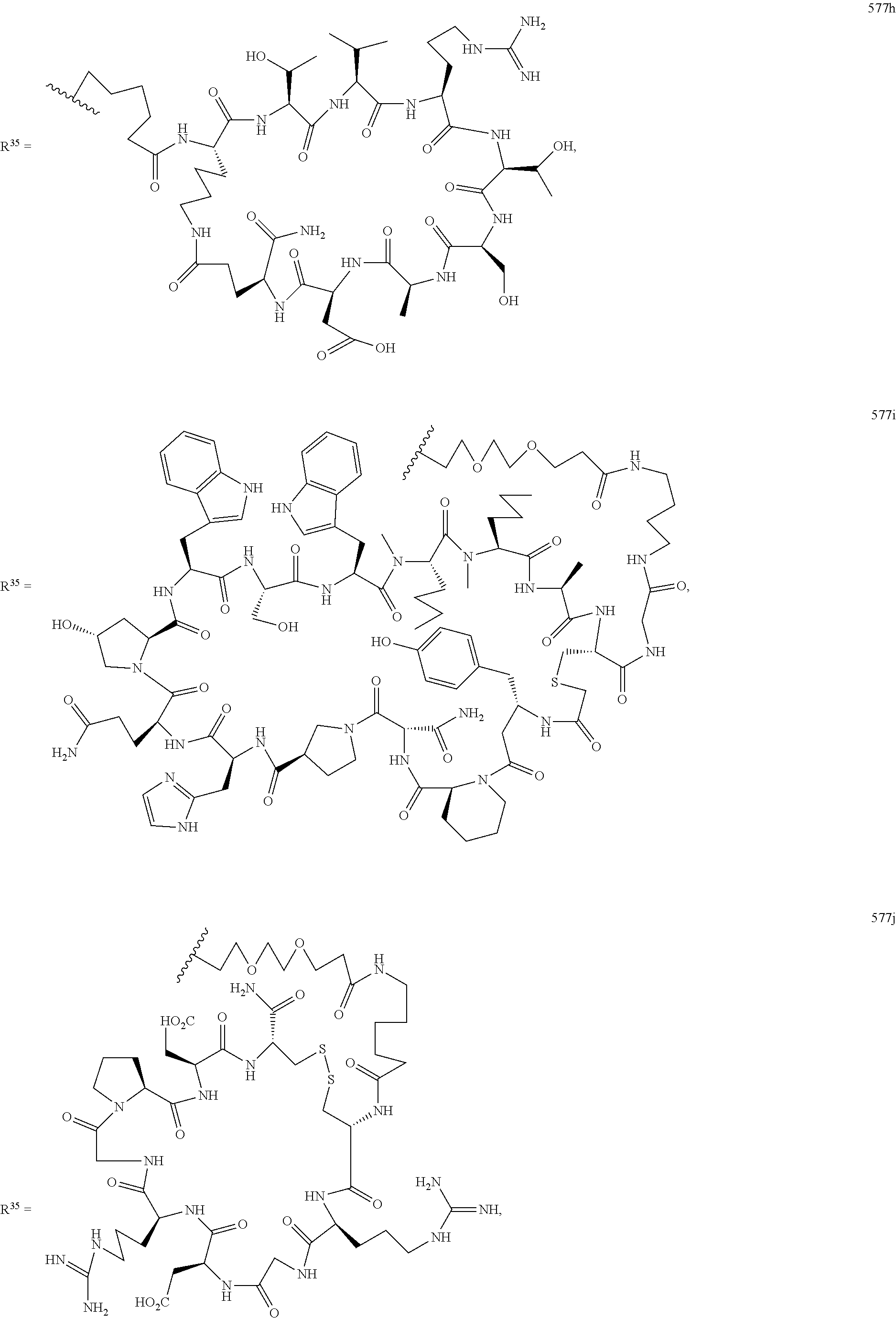

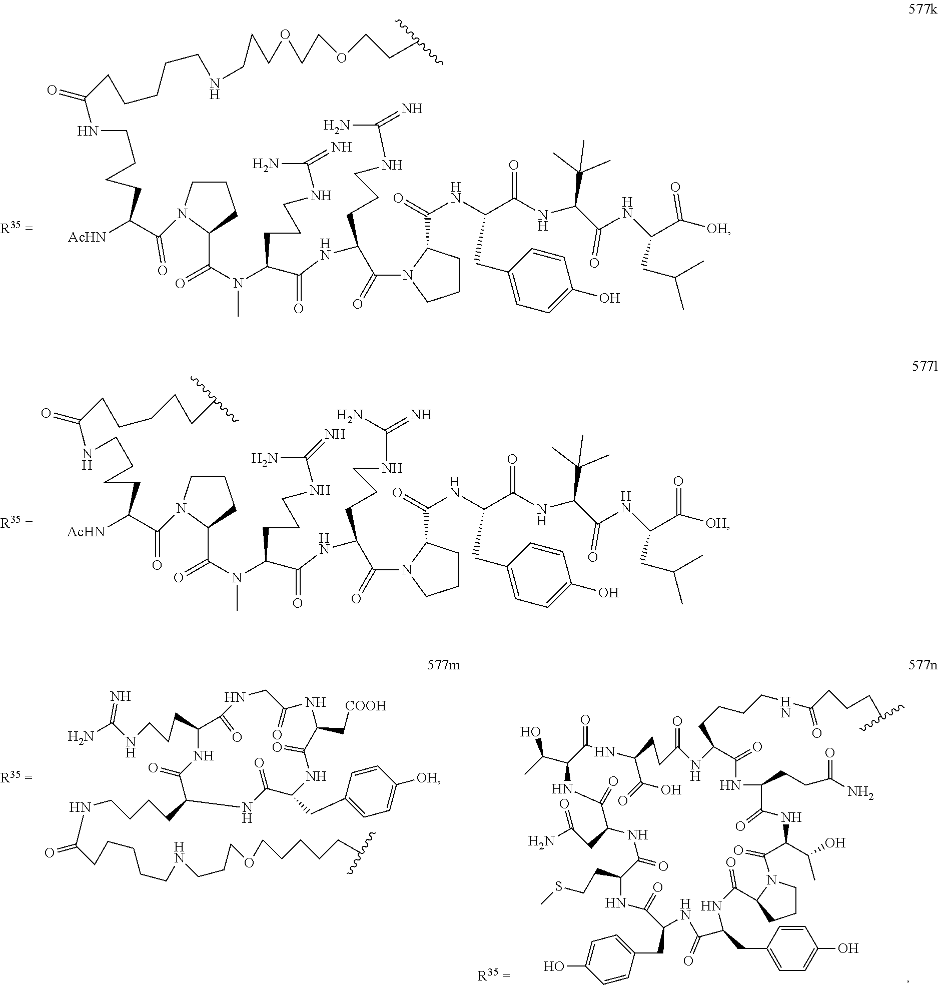

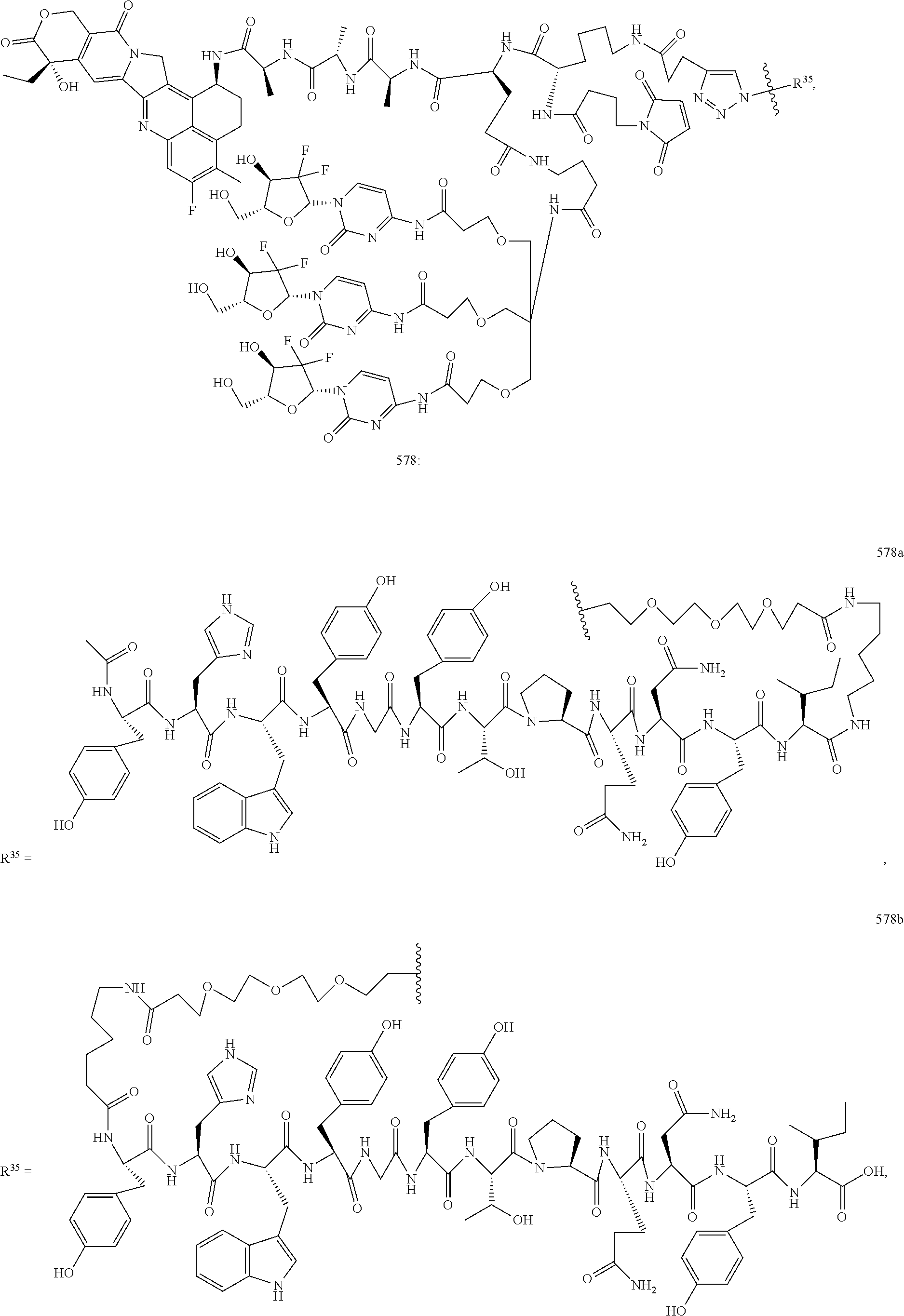

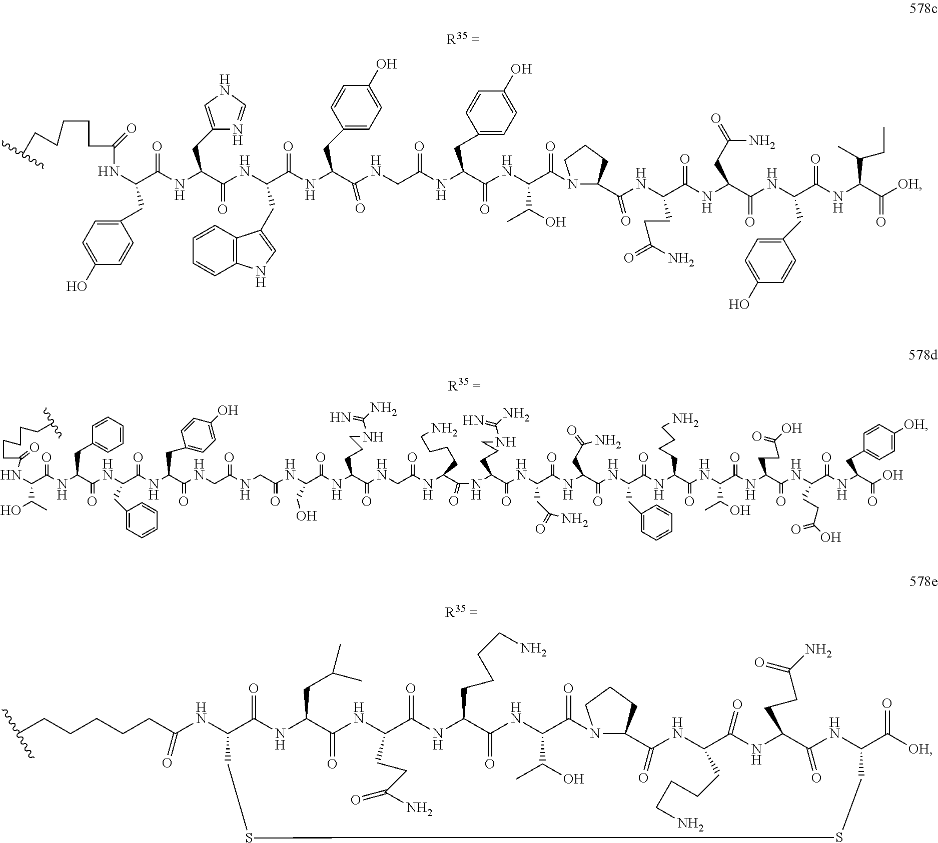

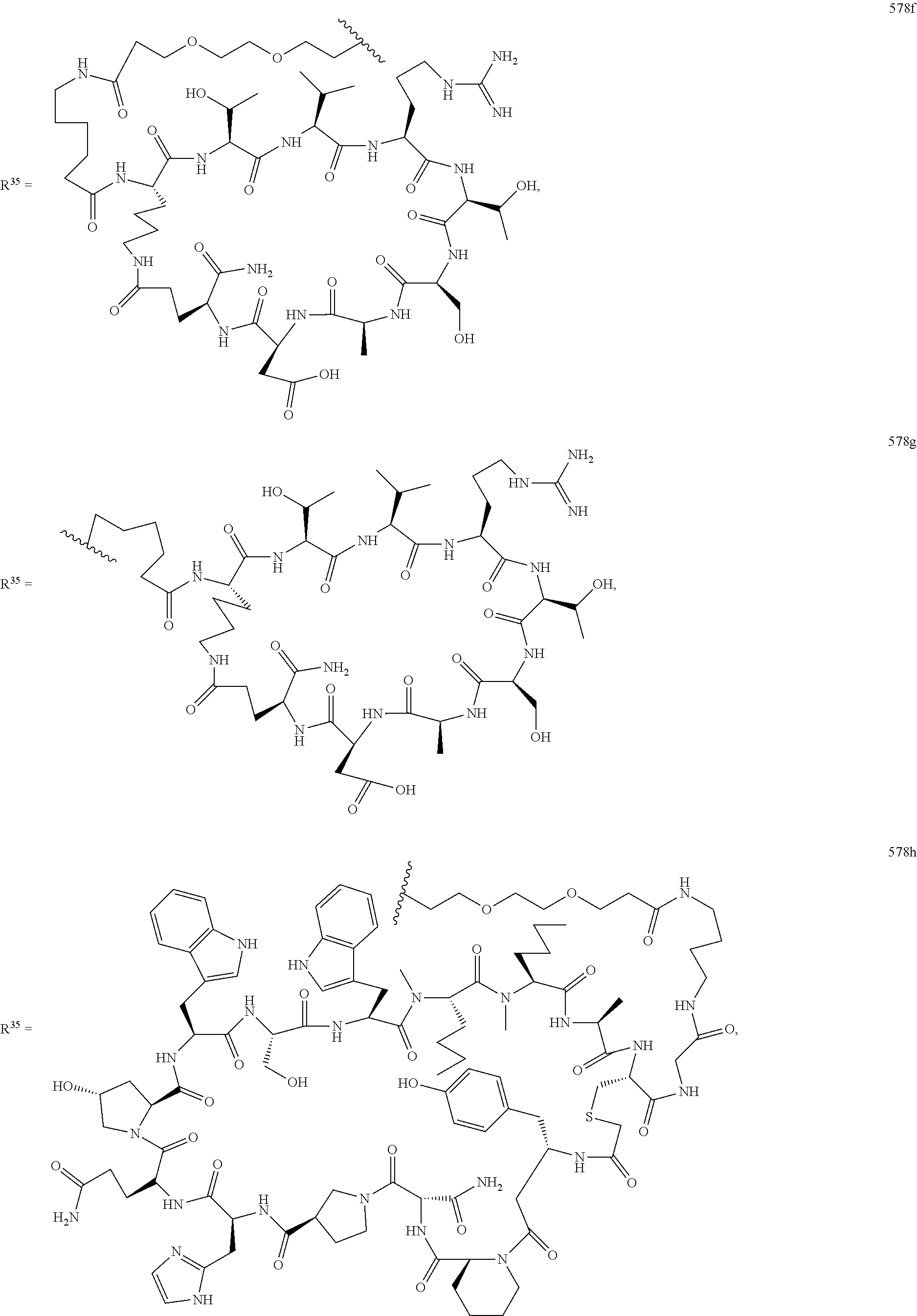

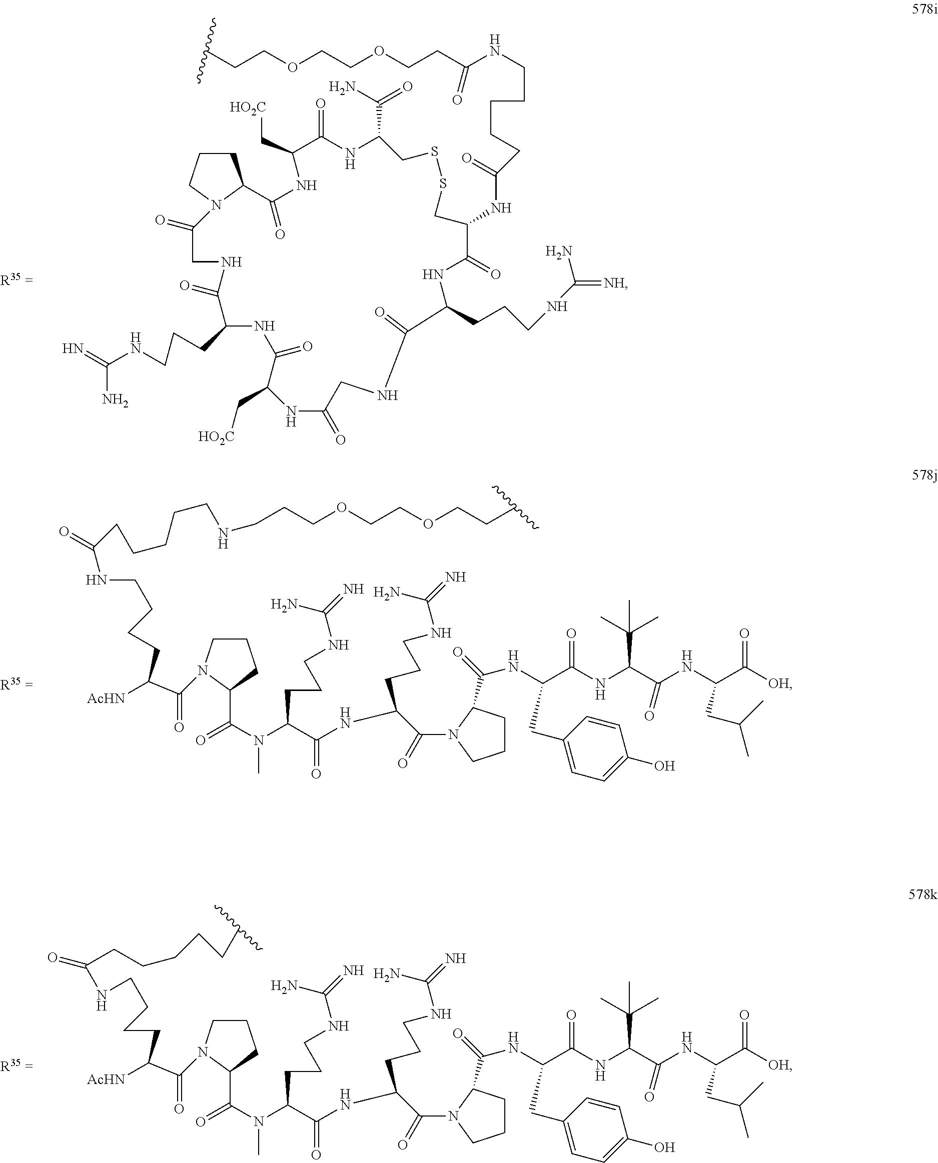

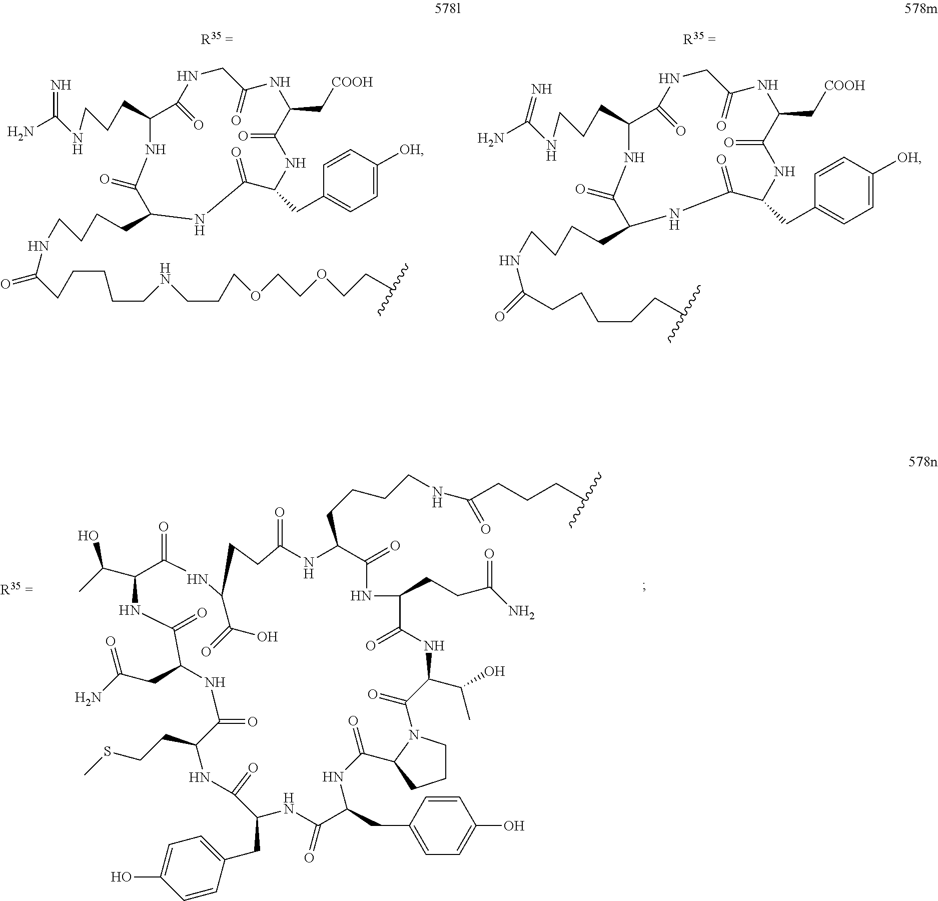 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

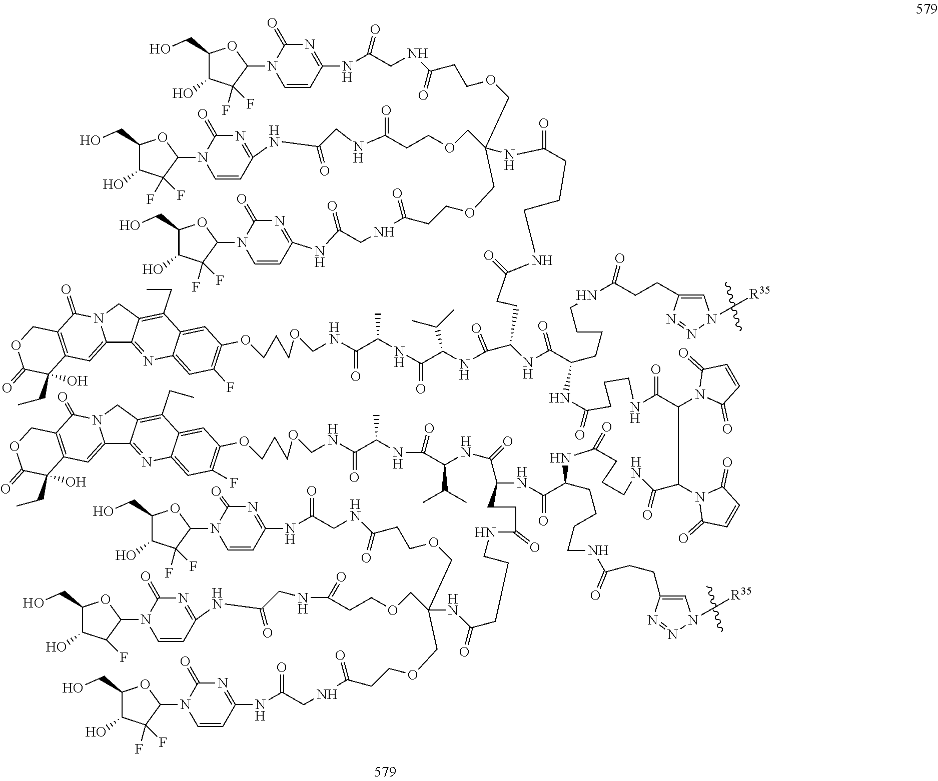

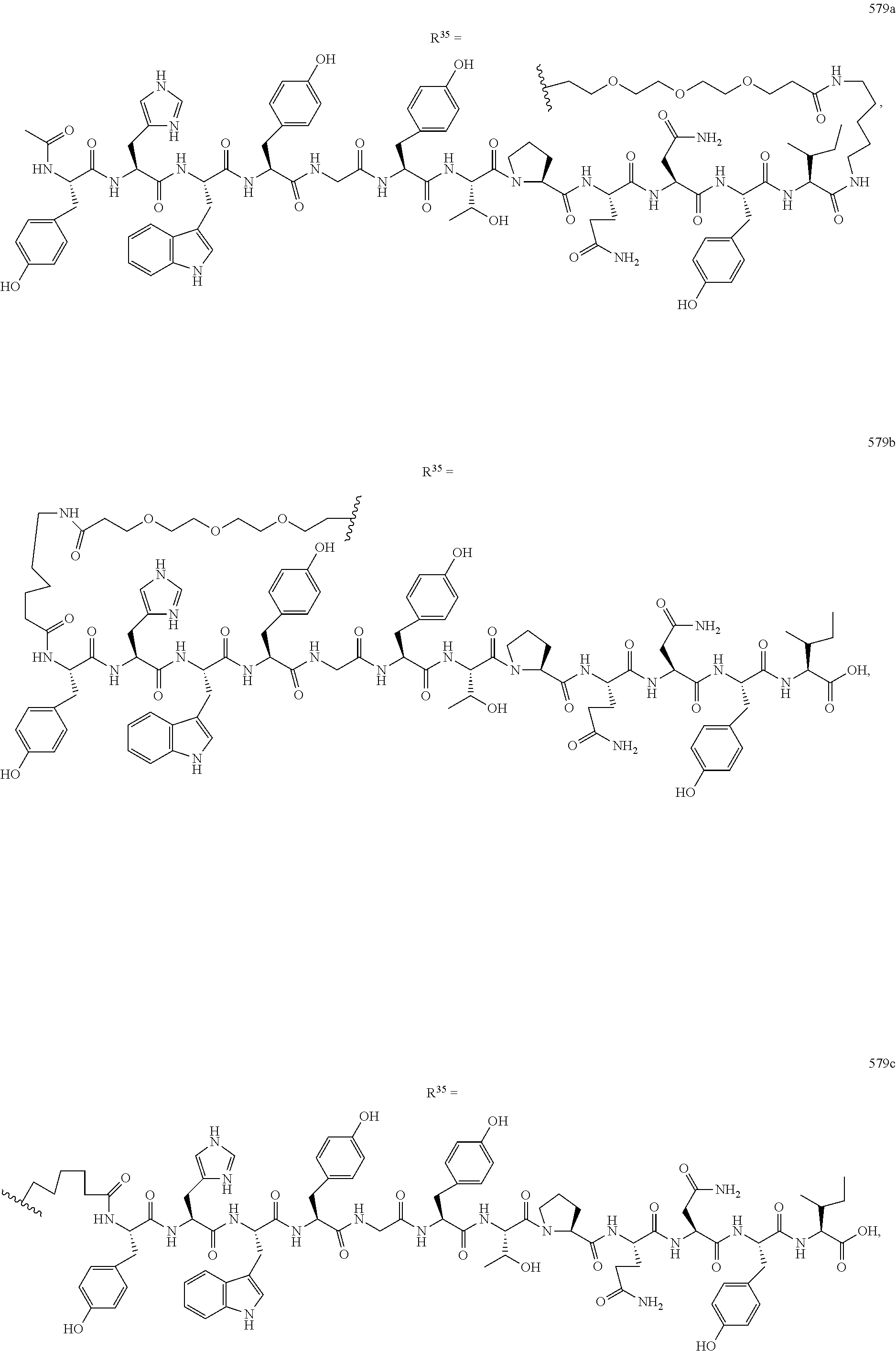

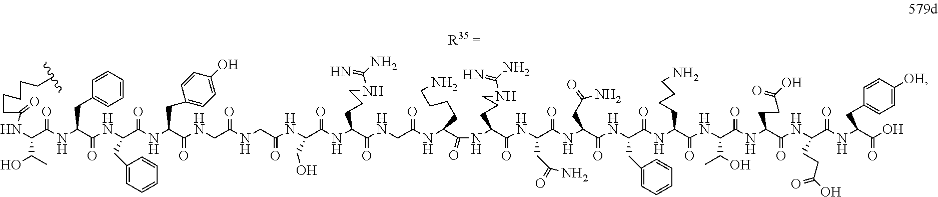 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

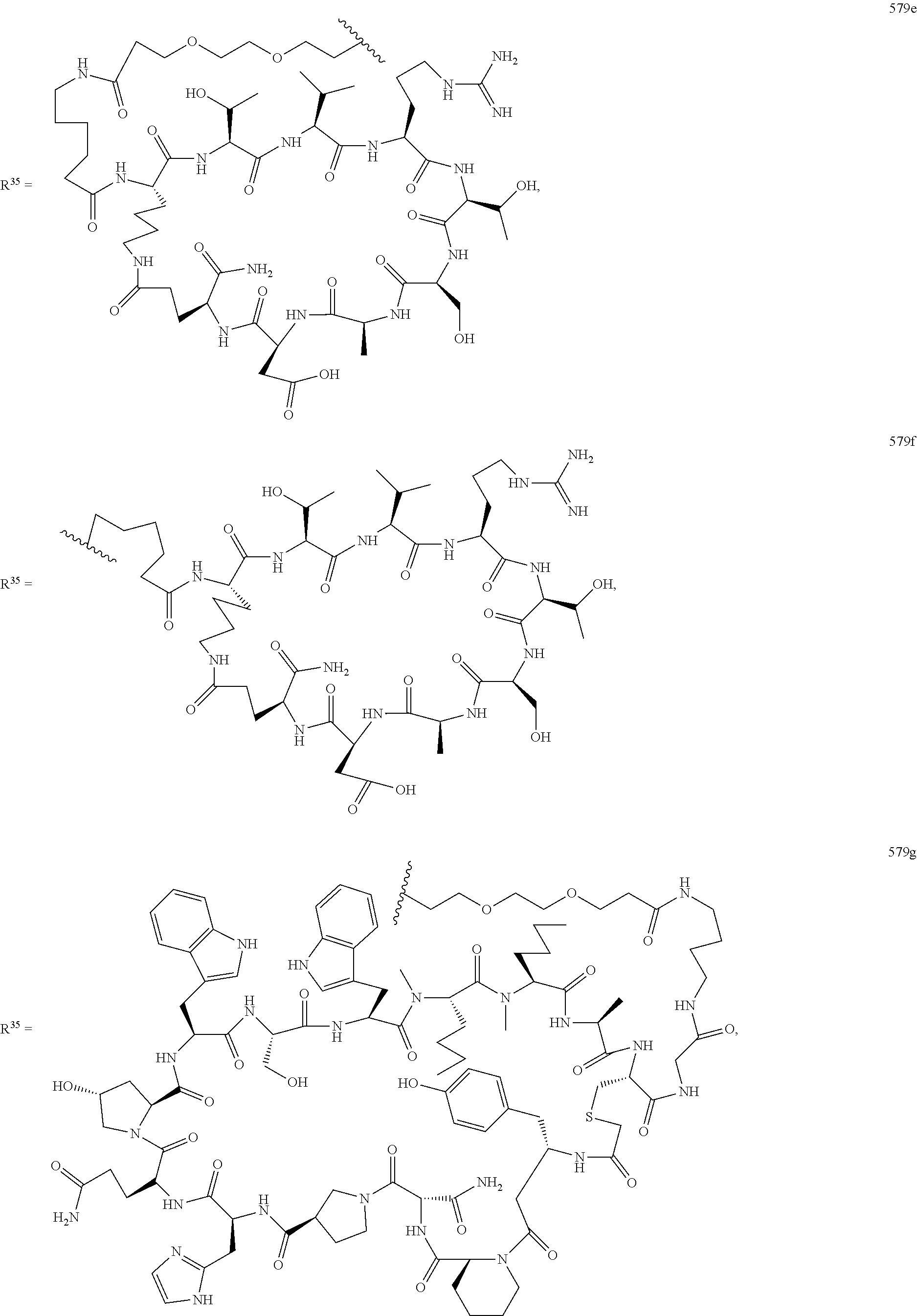

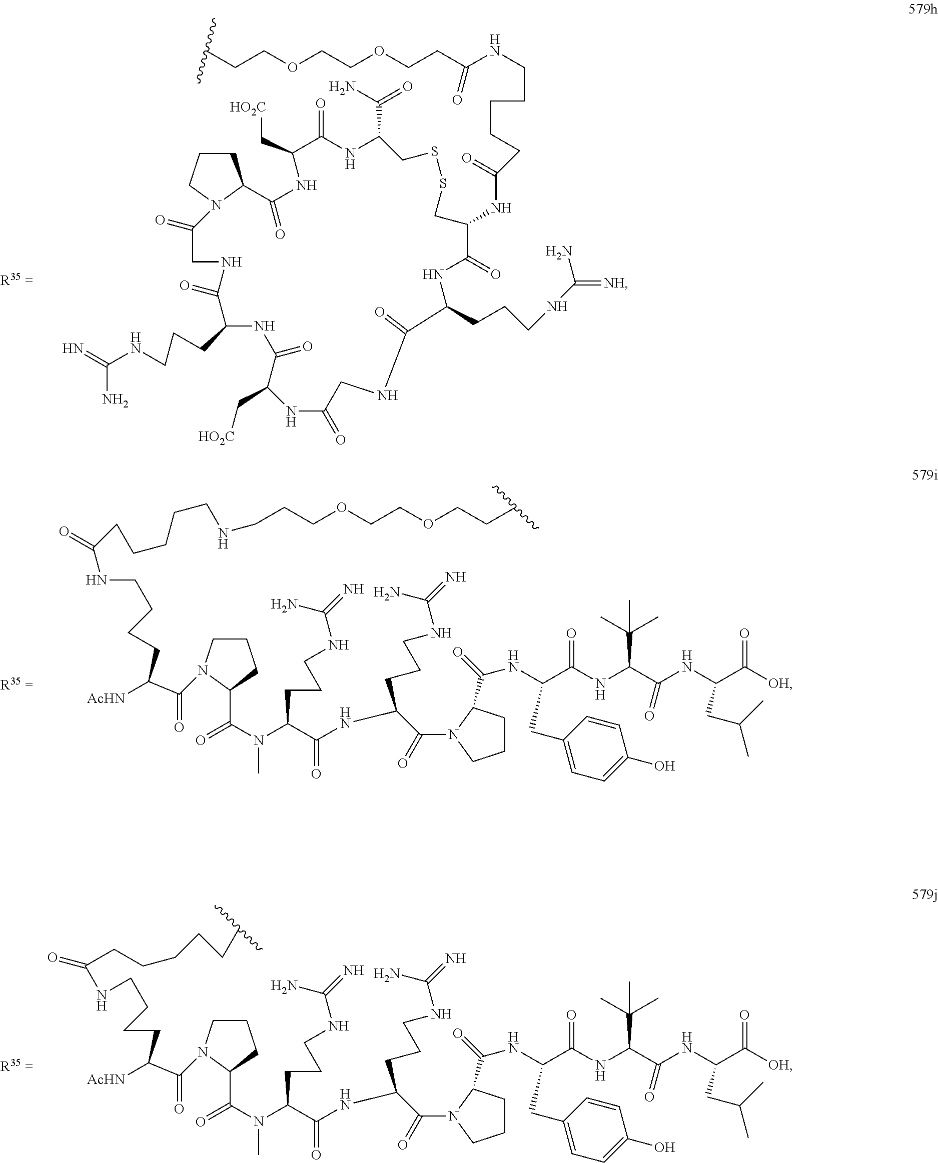

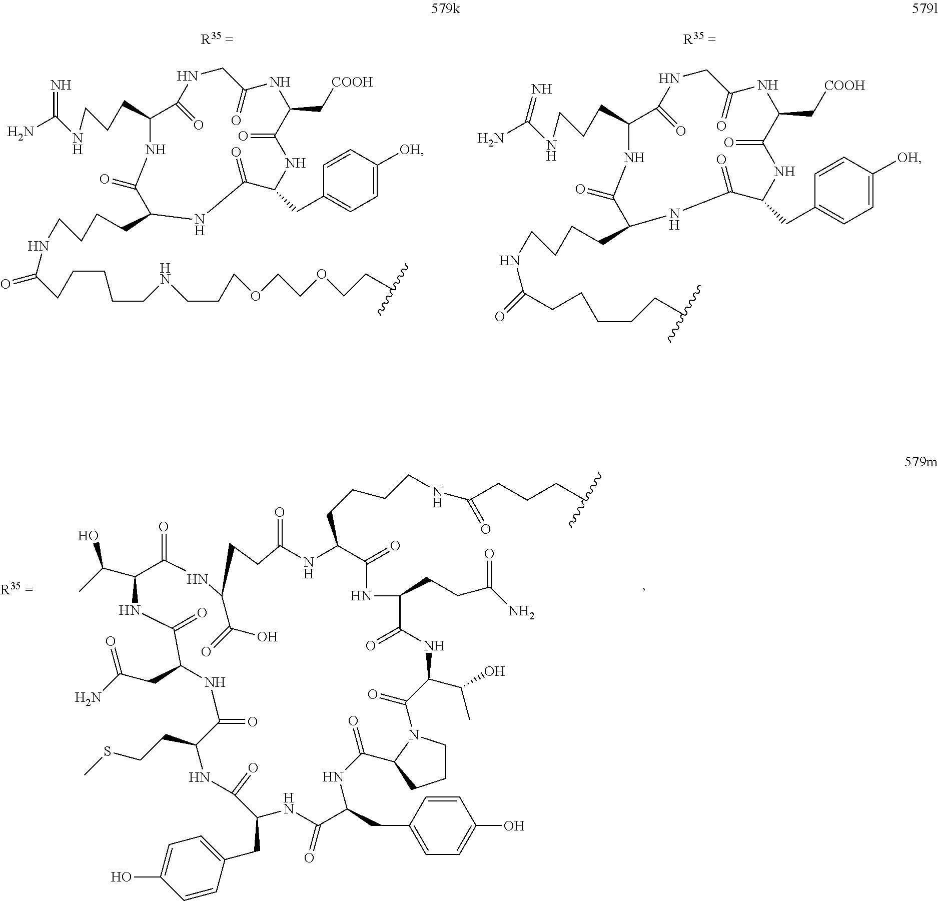 [SMILES: *CCOCCOCCCNCCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCCCC(=O)NCCCC[C@@H]1NC(=O)[C@@H](Cc2ccc(O)cc2)NC(=O)[C@H](CC(=O)O)NC(=O)CNC(=O)[C@H](CCCNC(=N)N)NC1=O ; *CCCC(=O)NCCCC[C@@H]1NC(=O)CC[C@@H](C(=O)O)NC(=O)[C@H]([C@@H](C)O)NC(=O)[C@H](CC(N)=O)NC(=O)[C@H](CCSC)NC(=O)[C@H](Cc2ccc(O)cc2)NC(=O)[C@H](Cc2ccc(O)cc2)NC(=O)[C@@H]2CCCN2C(=O)[C@H]([C@@H](C)O)NC(=O)[C@H](CCC(N)=O)NC1=O]

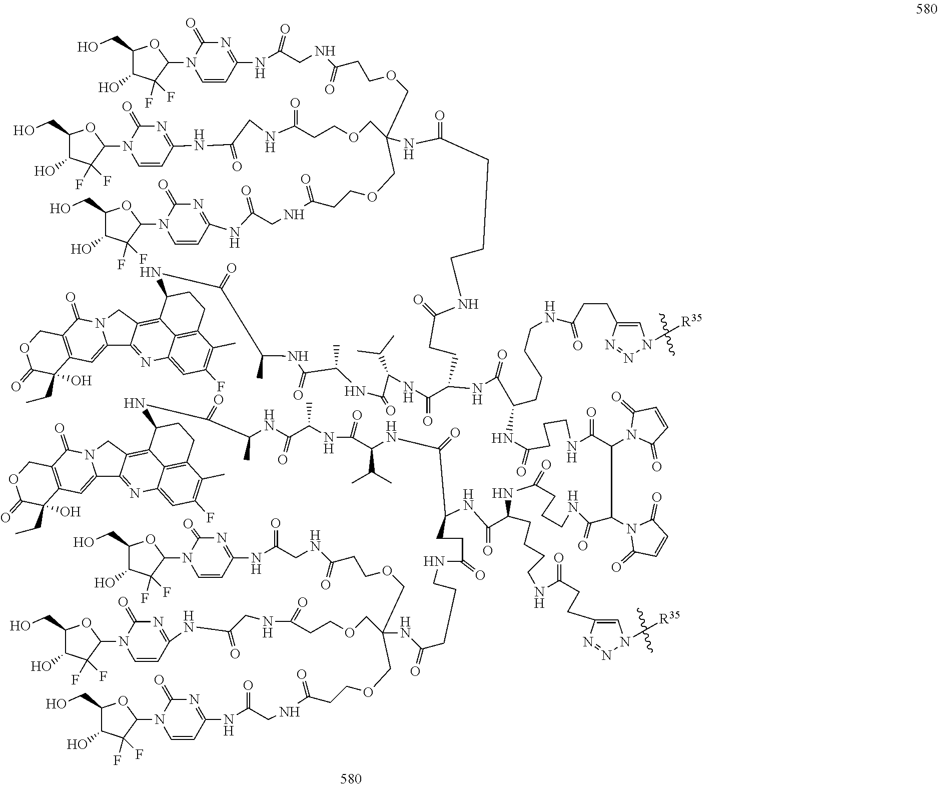

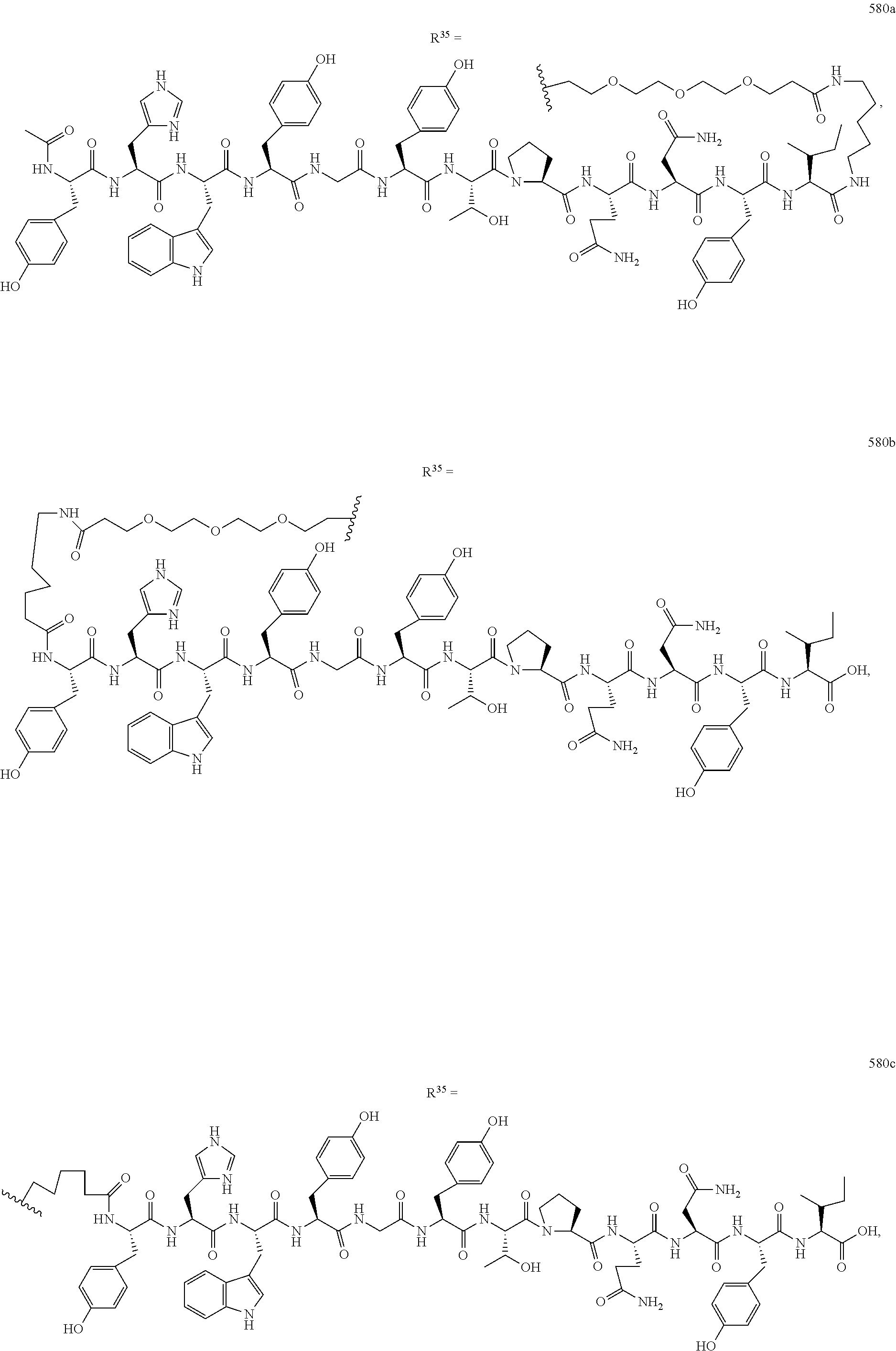

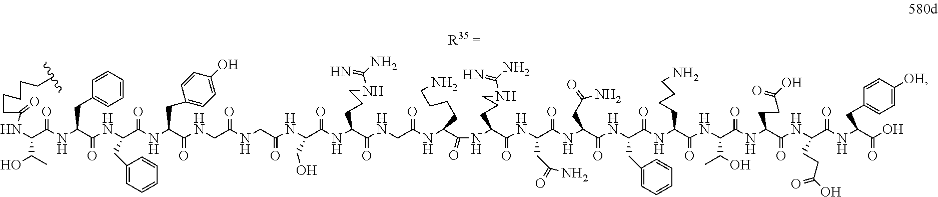 [SMILES: *CCCCCC(=O)N[C@H](C(=O)N[C@@H](Cc1ccccc1)C(=O)N[C@@H](Cc1ccccc1)C(=O)N[C@@H](Cc1ccc(O)cc1)C(=O)NCC(=O)NCC(=O)N[C@@H](CO)C(=O)N[C@@H](CCCNC(=N)N)C(=O)NCC(=O)N[C@@H](CCCCN)C(=O)N[C@@H](CCCNC(=N)N)C(=O)N[C@@H](CC(N)=O)C(=O)N[C@@H](CC(N)=O)C(=O)N[C@@H](Cc1ccccc1)C(=O)N[C@@H](CCCCN)C(=O)N[C@H](C(=O)N[C@@H](CCC(=O)O)C(=O)N[C@@H](CCC(=O)O)C(=O)N[C@@H](Cc1ccc(O)cc1)C(=O)O)C(C)O)C(C)O]

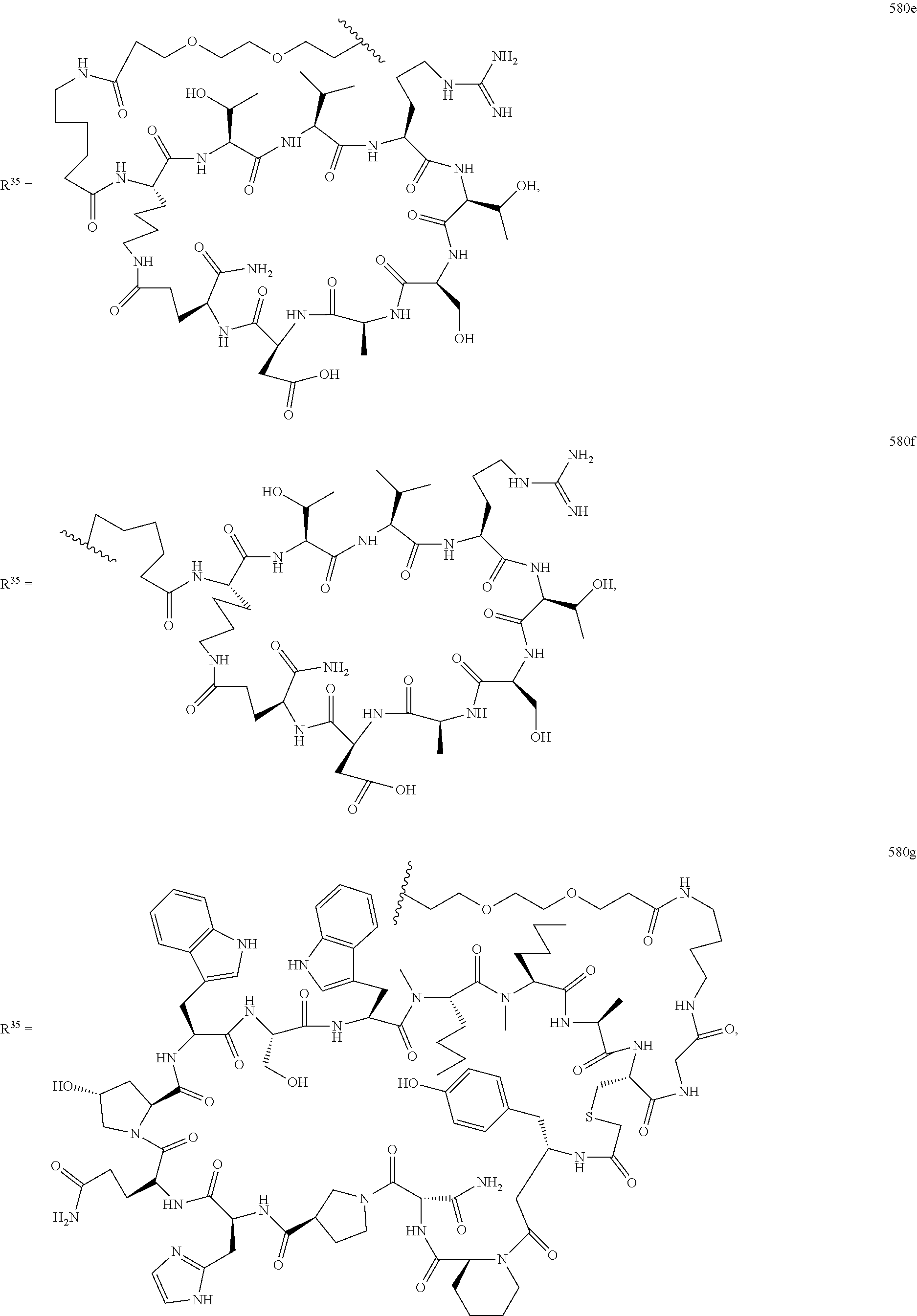

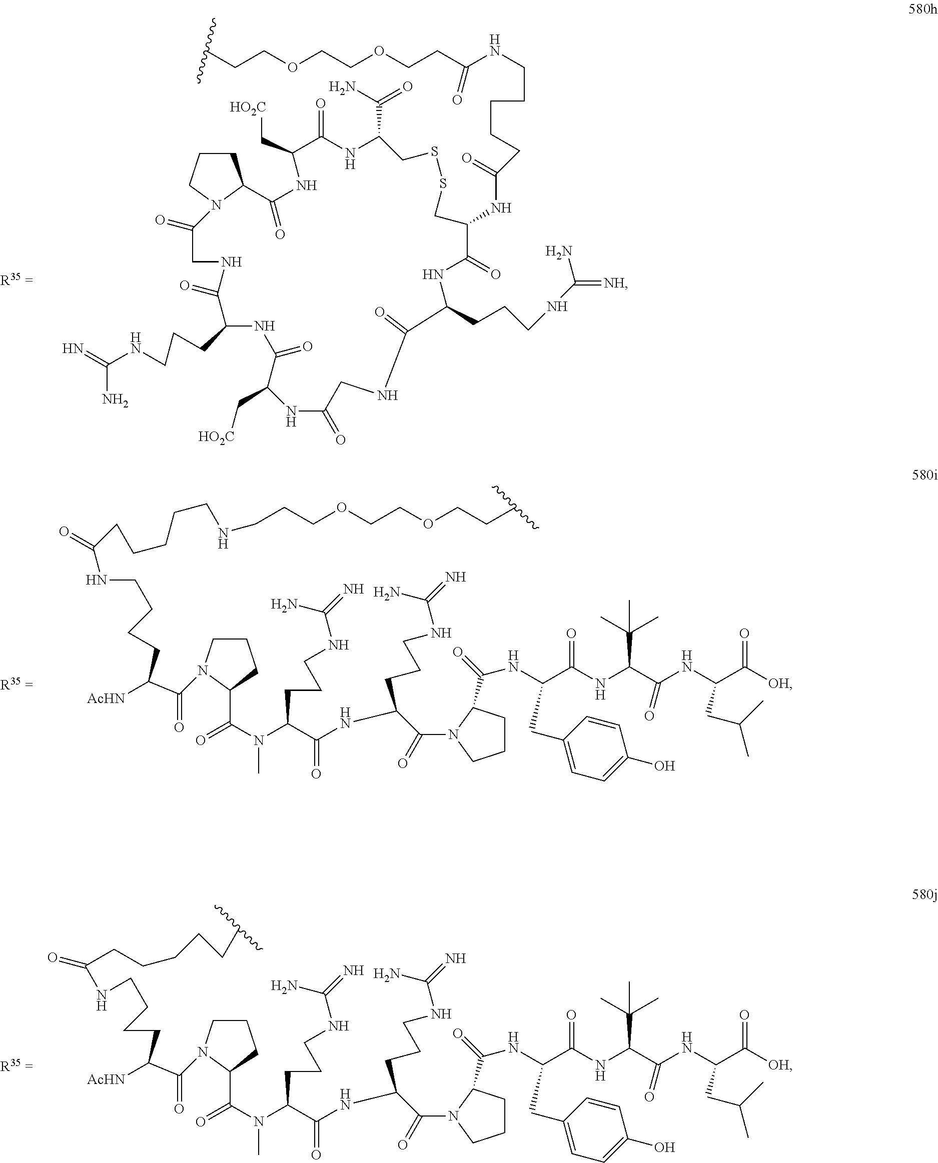

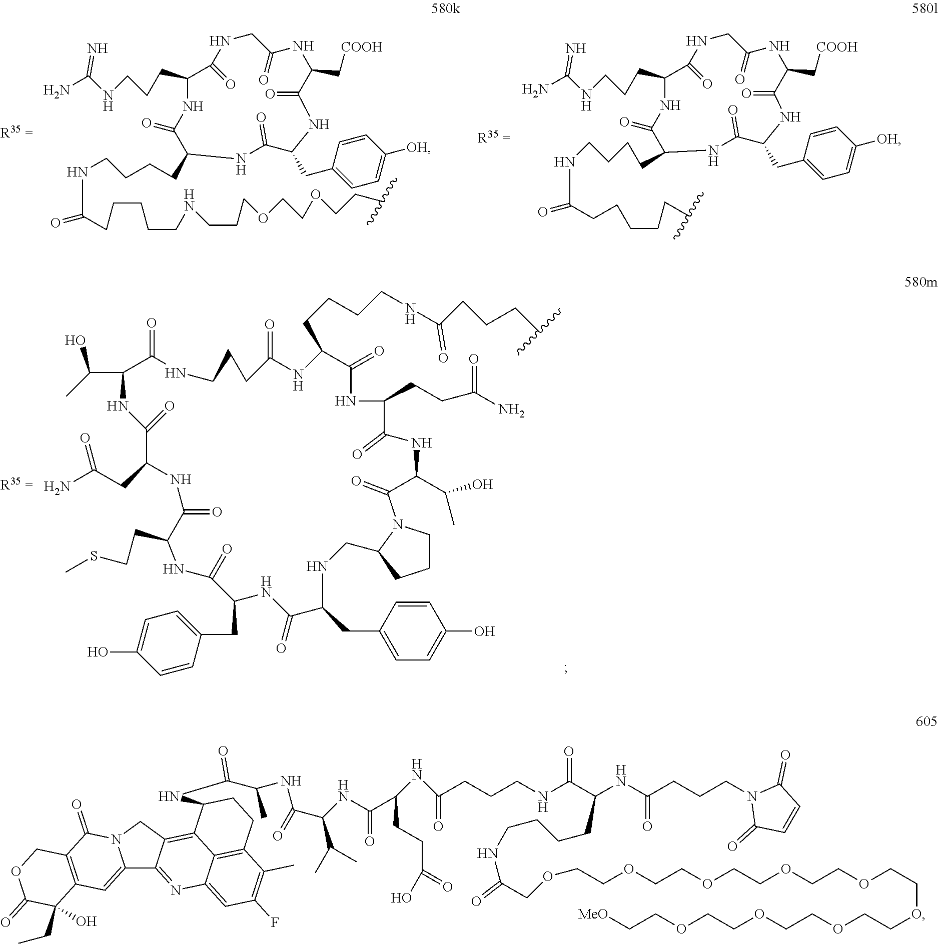

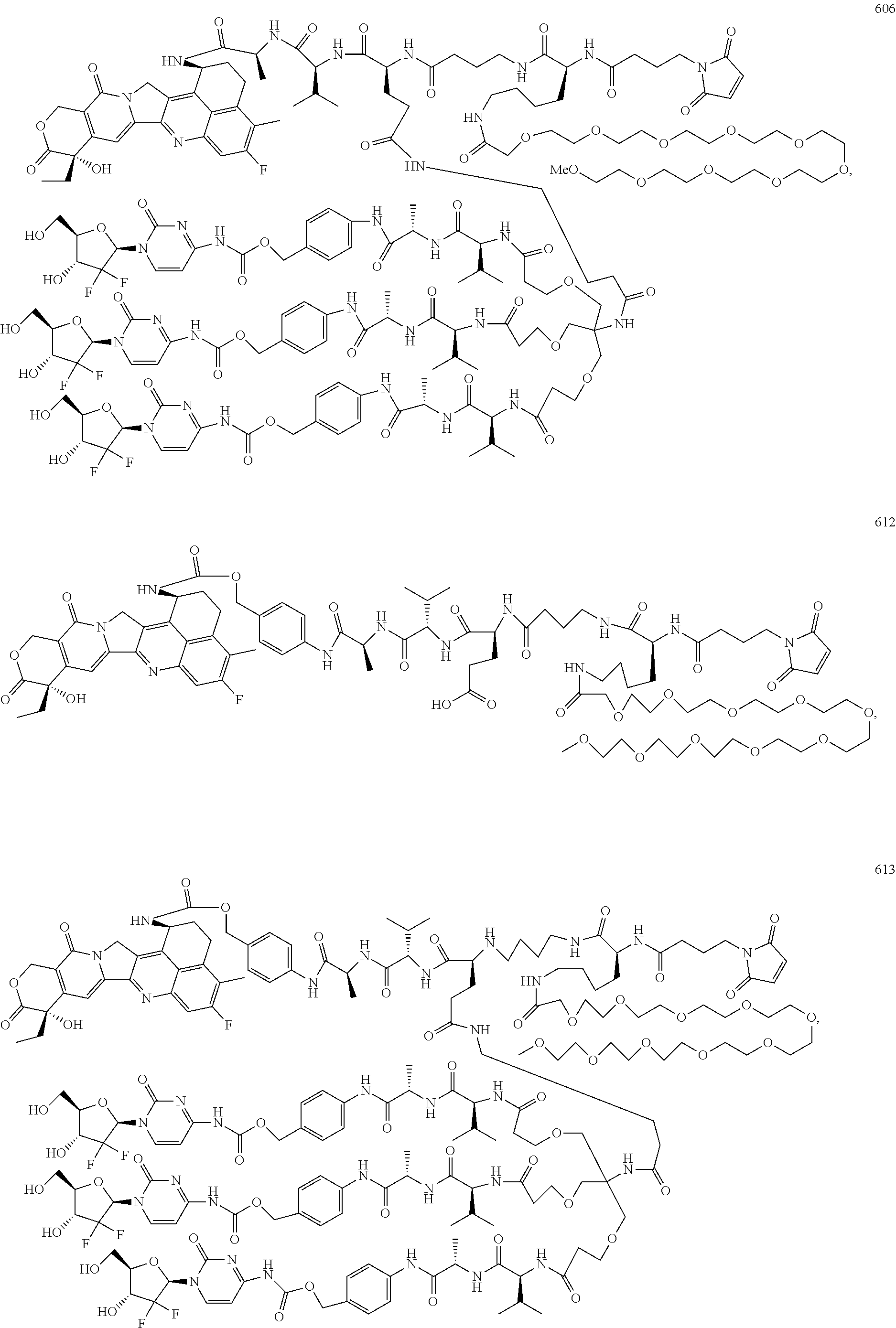

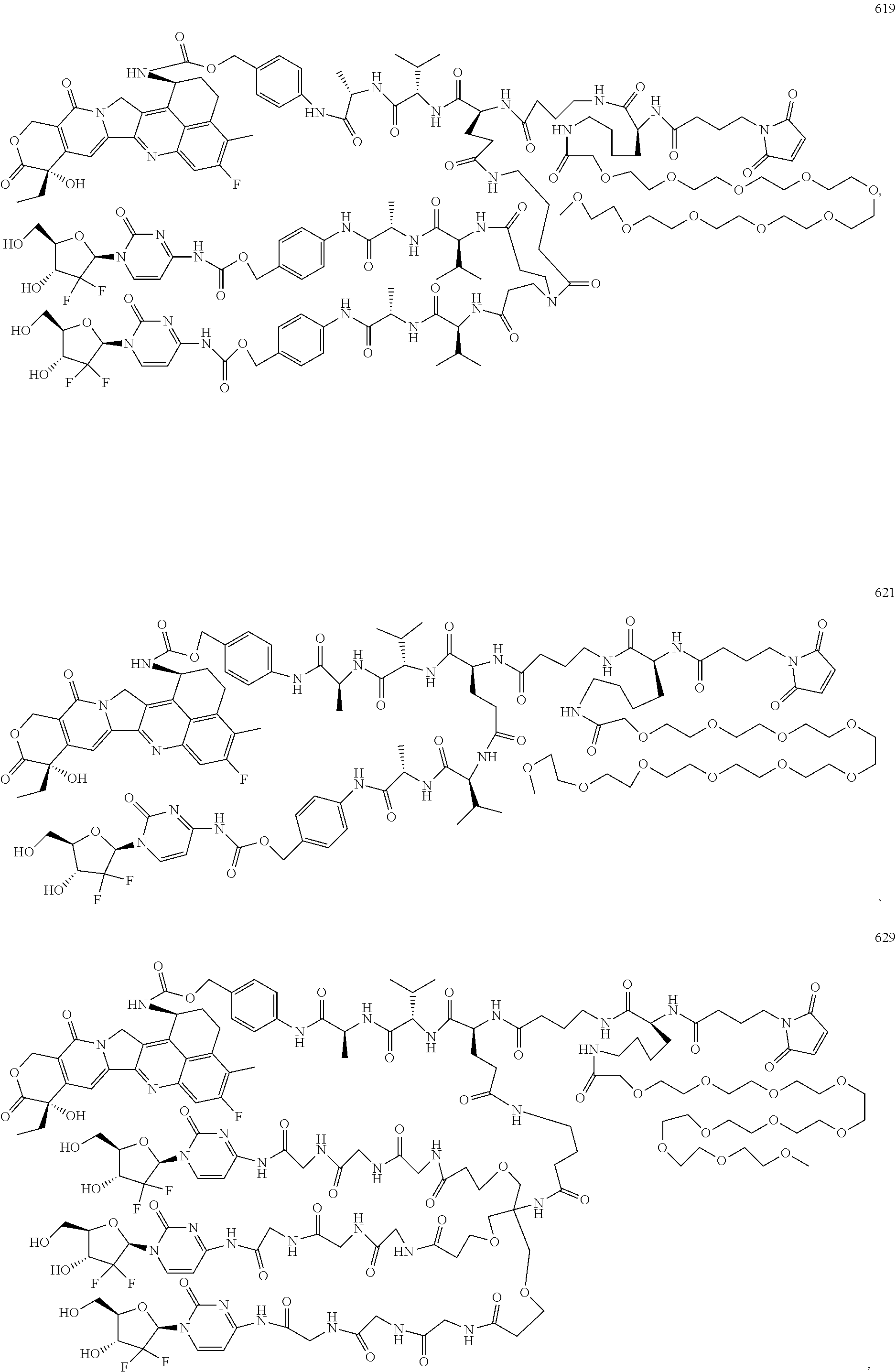

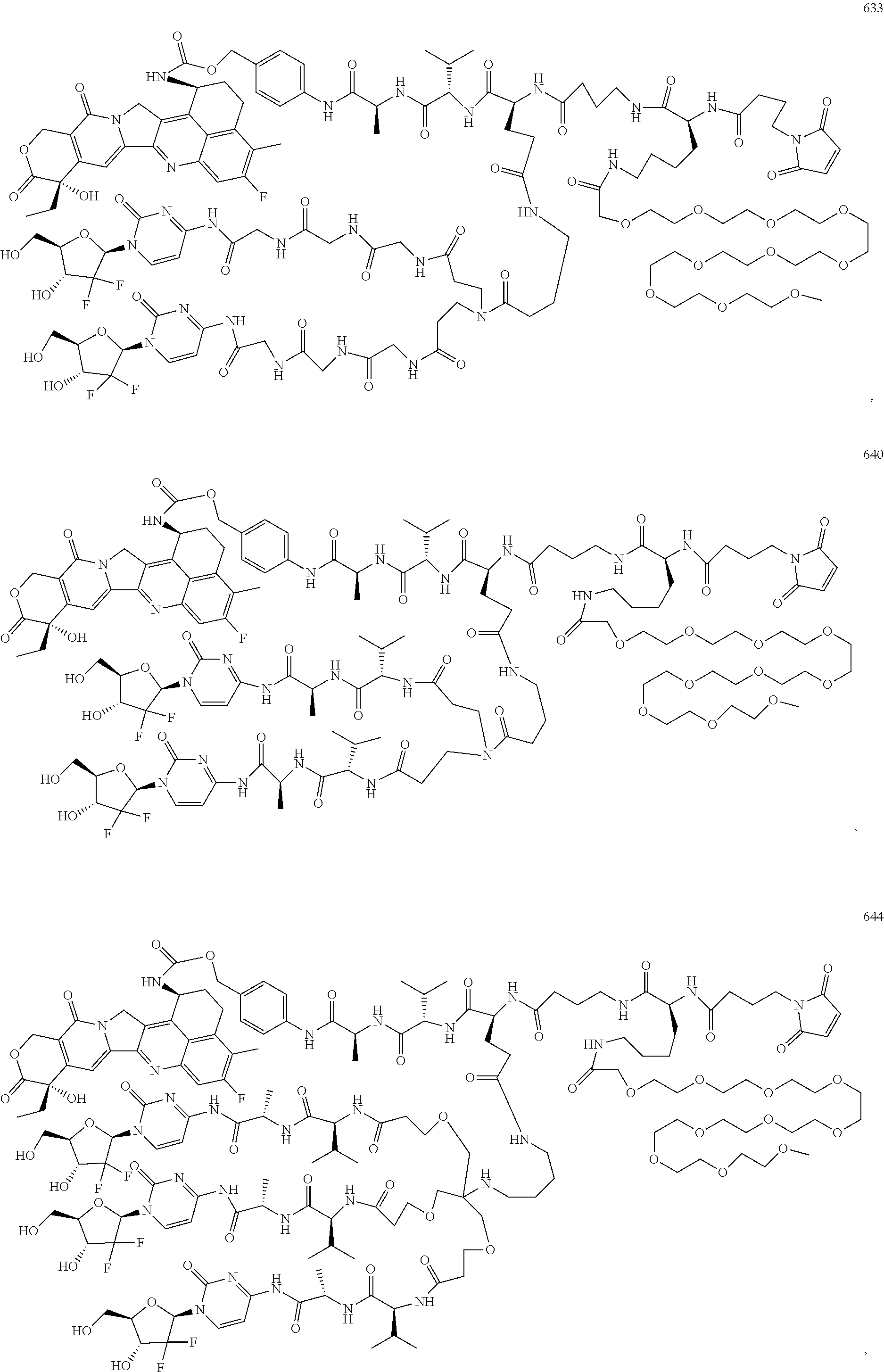

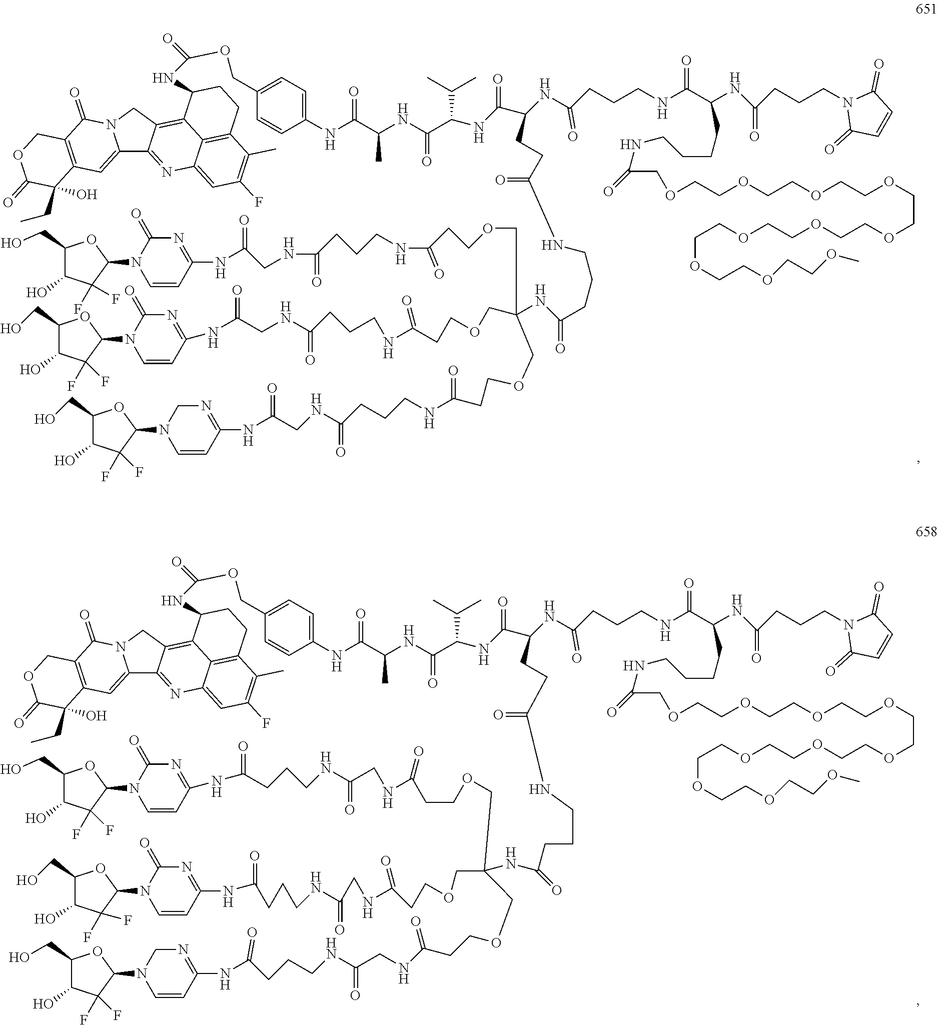

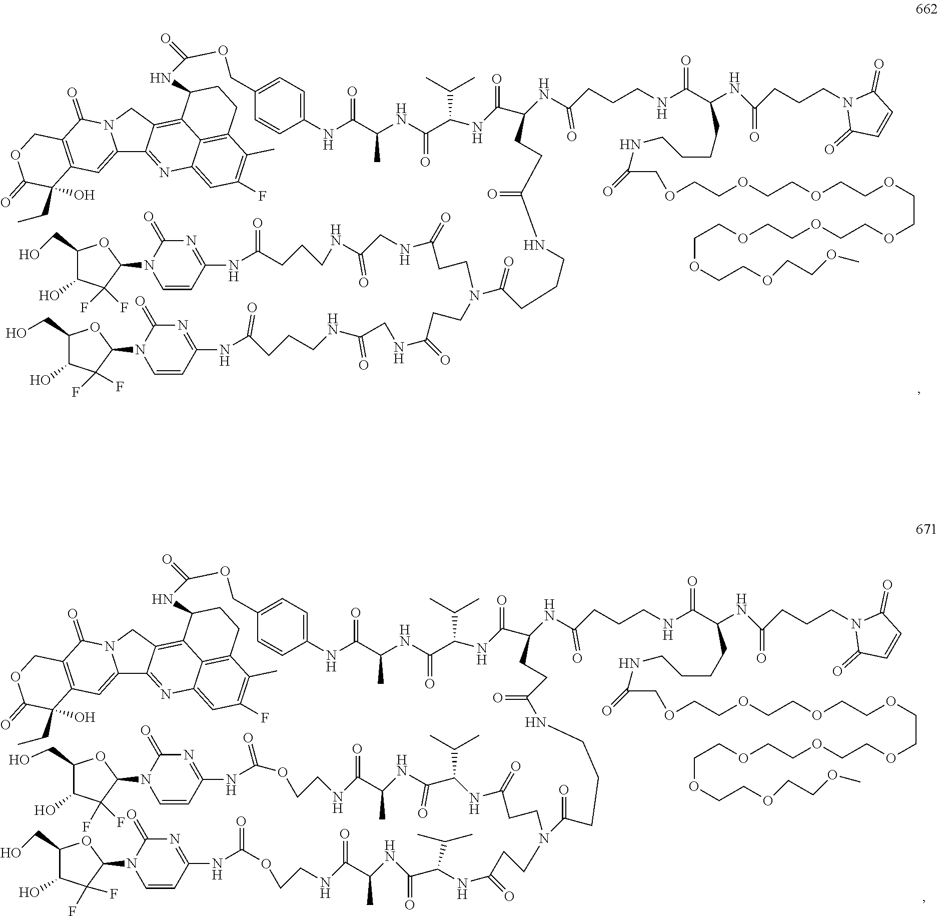

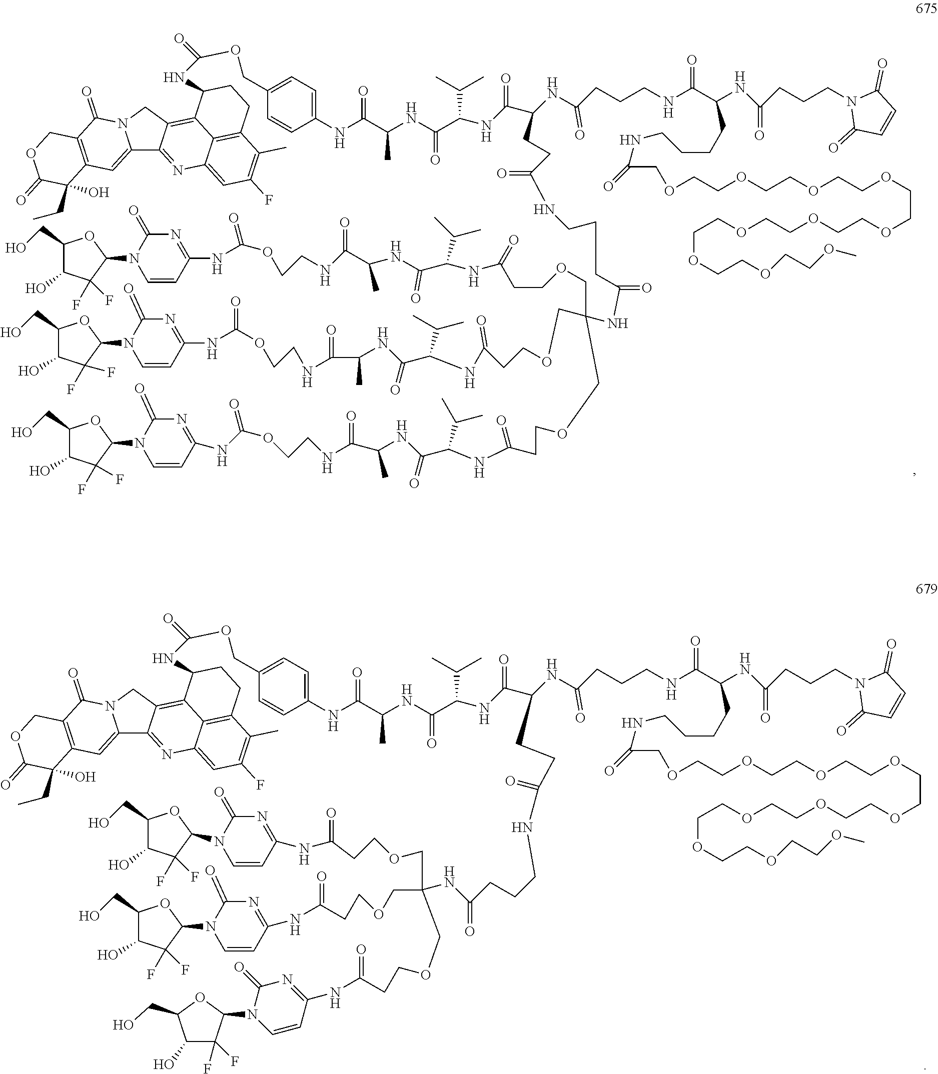

13. The antibody drug conjugate according to claim 1, wherein the antibody or the antibody like protein is selected from: one or several of a dAb, Fab, Fab′, F(ab′)₂, Fv, nanobody, diabody, triabody, tetrabody, miniantibody, a minibody, a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, a probody, a probody fragment, small immune proteins (SIP), a lymphokine, a hormone, a vitamin, a growth factor, a colony stimulating factor, a nutrient-transport molecule, large molecular weight proteins, fusion proteins, kinase inhibitors, gene-targeting agents, nanoparticles or polymers modified with antibodies or large molecular weight proteins; a vitamin (including folate); or large molecular peptides, a polymeric micelle, a liposome, a lipoprotein-based drug carrier, a nano-particle drug carrier, a dendrimer, and a particle said above coating or linking with a cell-binding ligand or a protein.

14. The antibody drug conjugate according to claim 1, wherein the antibody or the antibody like protein is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or any malfunctioned cells expressing any one of the following antigens or receptors: CD1, CD1a, CD1b, CD1c, CD1d, CD1e, CD2, CD3, CD3d, CD3e, CD3g, CD4, CD5,CD6, CD7, CD8, CD8a, CD8b, CD9, CD10, CD11a, CD11b, CD11c, CD 11d, CD12w, CD13, CD14, CD15, CD16, CD16a, CD16b, CDw17, CD18, CD19, CD20, CD21, CD22, CD23, CD24, CD25, CD26, CD27, CD28, CD29, CD30, CD31,CD32,CD32a, CD32b, CD33, CD34, CD35, CD36, CD37, CD38, CD39, CD40, CD41,CD42,CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55,CD56, CD57, CD58, CD59, CD60, CD60a, CD60b, CD60c, CD61,CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91,CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202(a, b), CD203, CD203c, CD204, CD205, CD206, CD207, CD208, CD209, CD210, CDw21Oa, CDw210b, CD211, CD212, CD213, CD213ai, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254,CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR₁, CRGF, CRIPTO, CXCR₅, LY64, TDGF1, 4-1BB, APO₂, ASLG659, BMPR1B, 4-1BB, 5AC, 5T4 (trophoblastic glycoprotein, TPBG, 5T4, Wnt-activated inhibitory factor 1 or WAIF1), adenocarcinoma antigen, AGS-5, AGS-22M6, activin receptor-like kinase 1, AFP, AKAP-4, ALK, alpha integrin, alpha v beta6,amino-peptidase N, Amyloid beta, androgen receptor, angiopoietin 2, angiopoietin 3, annexin A₁, anthrax toxin protective antigen, anti-transferrin receptor, AOC3 (VAP-1), B7-H₃, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, carbonic anhydrase IX, cardiac myosin, CCL11(C-C motif chemokine 11), CCR₄ (C-C chemokine receptor type 4), CCR₅, CD3E (epsilon), CEA (carcinoembryonic antigen), CEACAM3, CEACAM5 (carcino-embryonic antigen), CFD (Factor D), Ch4D5, cholecystokinin 2 (CCK2R), CLDN18 (Claudin-18), CLDN18.2 (Claudin-18.2), clumping factor A, cMet, CRIPTO, FCSFIR (colony stimulating factor 1 receptor), CSF2 (colony stimulating factor 2, granulocyte-macrophage colony-stimulating factor (GM-CSF)), CSP4, CTLA4 (cytotoxic T-lymphocyte-associated protein 4), CTAA16.88 tumor antigen, CXCR₄, C—X—C chemokine receptor type 4, cyclic ADP ribose hydrolase, cyclin B1, CYPIB1, cytomegalovirus, cytomegalovirus glycoprotein B, Dabigatran, DLL3 (delta-like-ligand 3), DLL4 (delta-like-ligand 4), DPP4 (dipeptidyl-peptidase 4), DR5 (feath receptor 5), E. coli shiga toxin type-1, E. coli shiga toxin type-2, ED-B, EGFL7 (EGF-like domain-containing protein 7), EGFR, EGFRII, EGFRvIII, endoglin, endothelin B receptor, endotoxin, EpCAM (epithelial cell adhesion molecule), EphA2, Episialin, ERBB2 (epidermal growth factor receptor 2), ERBB3, ERG (TMPRSS₂ ETS fusion gene), Escherichia coli, ETV6-AML, FAP (fibroblast activation protein alpha), FCGR₁, alpha-Fetoprotein, Fibrin II, beta chain, fibronectin extra domain-B, FOLR (folate receptor), folate receptor alpha, folate hydrolase, Fos-related antigen iF protein of respiratory syncytial virus, frizzled receptor, fucosyl GM1, GD₂ gangl105ide, G-28 (a cell surface antigen glyvolipid), GD3 idiotype, GloboH, glypican 3, N-glycolylneuraminic acid, GM3, GMCSF receptor a-chain, growth differentiation factor 8, GP100, GPNMB (trans-membrane glycoprotein NMB), GUCY2C (guanylate cyclase 2C, guanylyl cyclase C(GC-C), intestinal fuanylate cyclase, fuanylate cyclase-C receptor, heat-stable enterotoxin receptor (hSTAR)), heat shock proteins, hemagglutinin, hepatitis B surface antigen, hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, histone complex, HLA-DR (human leukocyte antigen), HLA-DR10, HLA-DRB, HMWMAA, human chorionic gonadotropin, HNGF, human scatter factor receptor kinase, HPV E6/E7, Hsp90, hTERT, ICAM-1 (Intercellular Adhesion Molecule 1), idiotype, IGF1R (IGF-1, insulin-like growth factor 1 receptor), IGHE, IFN-γ, Influenza hemagglutinin, IgE, IgF Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (insulin-like growth factor 2), Integrins (a4, am>p3, αvP3, a4p7, a5 μl, a6p4, a7p7, a11p3, a5p5, αᵥβ5), interferon gamma-induced protein, ITGA2, ITGB2, KIR2D, Kappa Ig, LCK, Le, Legumain, Lewis-Y antigen, LFA-1 (lymphocyte function-associated antigen 1, CD11a), LHRH, LINGO—1, lipoteichoic acid, LIVIA, LMP2, LTA, MAD-CT-1, MAD-CT-2, MAGE-1, MAGE-2, MAGE-3, MAGE A₁, MAGE A₃, MAGE 4, MART1, MCP-1, MIF (macrophage migration inhibitory factor, or glycosylation-inhibiting factor (GIF)), MS4A₁ (membrane-spanning 4-domains subfamily A member 1), MSLN (mesothelin), MUC1(Mucin 1, cell surface associated (MUC1) or polymorphic epithelial mucin (PEM)), MUC1-KLH, MUC16 (CA125), MCP1(monocyte chemotactic protein 1), MelanA/MART1, ML-IAP, MPG, MS4A₁ (membrane-spanning 4-domains subfamily A), MYCN, myelin-associated glycoprotein, myostatin, NA17, NARP-1, NCA-90 (granulocyte antigen), Nectin-4 (ASG-22ME), NGF,neural apoptosis-regulated proteinase 1, NOGO-A, Notch receptor, nucleolin, neu oncogene product, NY-BR-1, NY-ESO—1, OX-40, OxLDL (oxidized low-density lipoprotein), OY-TES1, P21, p⁵³ nonmutant, P97, Page4, PAP, paratope of anti-(N-glycolylneuraminic acid), PAX3, PAX5, PCSK9, PDCD1 (PD-1, programmed cell death protein 1), PDGF-Ra (Alpha-type platelet-derived growth factor receptor), PDGFR-p, PDL-1, PLAC1, PLAP-like testicular alkaline phosphatase, platelet-derived growth factor receptor beta, phosphate-sodium co-transporter, PMEL 17, polysialic acid, proteinase3 (PR1), prostatic carcinoma, PS (Phosphatidylserine), prostatic carcinoma cells, Pseudomonas aeruginosa, PSMA, PSA, PSCA, rabies virus glycoprotein, RHD (Rh polypeptide 1 (RhPI)), Rhesus factor, RANKL, RhoC, Ras mutant, RGS5, ROBO4, respiratory syncytial virus, RON, ROR₁, Sarcoma translocation breakpoints, SART3, sclerostin, SLAMF7 (SLAM family member 7), Selectin P, SDC1 (Syndecan 1), sLe(a), Somatomedin C, SIP (Sphingosine-1-phosphate), Somatostatin, sperm protein 17, SSX2, STEAPI (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (transforming growth factor beta), TGF-β1, TGF-β2 (transforming growth factor-beta 2), Tie (CD202b), Tie2, TIM-1 (CDX-014), Tn, TNF, TNF-α, TNFRSF8, TNFRSFlOB (tumor necrosis factor receptor superfamily member 10B), TNFRSF-13B (tumor necrosis factor receptor superfamily member 13B), TPBG (trophoblast glycoprotein), TRAIL-R₁ (tumor necrosis apoptosis inducing ligand receptor 1), TRAILR2 (death receptor 5 (DR5)), tumor-associated calcium signal transducer 2, tumor specific glycosylation of MUC1, TWEAK receptor, TYRPI(glycoprotein 75), TRP-1 (Trop-1), TRP-2 (Trop-2), tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

15. The drug conjugate according to claim 14, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, or any cells that grow and divide at an unregulated, quickened pace to cause cancers.

16. A pharmaceutical composition comprising a therapeutically effective amount of the antibody drug conjugate of claim 1, and a pharmaceutically acceptable salt, carrier, diluent, or excipient therefore, or a combination of the conjugates thereof, for use in the treatment or prevention of a cancer.

17. The pharmaceutical composition according to claim 16, is either in the liquid formula or in the formulated lyophilized solid, comprising by weight of: 0.01%-99% of one or more of the antibody drug conjugates 0.0%-20.0% of one or more polyols; 0.0%-2.0% of one or more surfactants; 0.0% -5.0% of one or more preservatives; 0.0% -30% of one or more amino acids; 0.0% -5.0% of one or more antioxidants; 0.0% -0.3% of one or more metal chelating agents; 0.0% -0.5% of hyaluronidase with activity of >500 u/mg; 0.0% -30.0% of one or more buffer salts for adjusting pH of the formulation to pH 4.5 to 7.5; and 0.0% -30.0% of one or more of isotonic agent for adjusting osmotic pressure between about 250 to 350 mOsm when reconstituted for administration to a patient;
wherein the polyol is selected from fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts);
wherein the surfactant is selected from polysorbate 20, polysorbate 40, polysorbate 65, polysorbate 80, polysorbate 81, or polysorbate 85, poloxamer, poly(ethylene oxide)-poly(propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is selected from benzyl alcohol, octadecyldimethylbenzyl ammonium chloride, hexamethonium chloride, benzalkonium chloride, benzethonium chloride, phenol, butyl and benzyl alcohol, alkyl parabens such as methyl or propyl paraben, catechol, resorcinol, cyclohexanol, 3-pentanol, or m-cresol;
wherein the amino acid is selected from arginine, cystine, glycine, lysine, histidine, ornithine, isoleucine, leucine, alanine, glycine glutamic acid or aspartic acid;
wherein the antioxidant is selected from ascorbic acid, glutathione, cystine or and methionine;
wherein the chelating agent is selected from EDTA or EGTA;
wherein the buffer salt is selected from sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salts;
wherein the tonicity agent is selected from mannitol, sorbitol, sodium acetate, potassium chloride, sodium phosphate, potassium phosphate, trisodium citrate, or sodium chloride.

18. The pharmaceutical composition according to claim 16, which is packed in a vial, bottle, pre-filled syringe, or pre-filled auto-injector syringe, in a form of a liquid or lyophilized solid.

19. The antibody drug conjugate of claim 1, having in vitro, in vivo or ex vivo cell killing activity.

20. A-Th pharmaceutical composition according to claim 16, wherein the pharmaceutical composition is administered concurrently with a chemotherapeutic agent, a radiation therapy, an immunotherapy agent, an autoimmune disorder agent, an anti-infectious agent or the other conjugates for use in the synergistical treatment or prevention of a cancer.

21. The pharmaceutical composition according to claim 20, wherein the chemotherapeutic is selected from:
(1). a). an alkylating agent: selected from nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: including busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). A plant alkaloid: selected from the group consisting of Vinca alkaloids: including vincristine, vinblastine, vindesine, vinorelbine, and navelbin; Taxoids: comprising paclitaxel, docetaxol and their analogs, Maytansinoids including DM1, DM2, DM3, DM4, DM5, DM6, DM7, maytansine, ansamitocins and their analogs, cryptophycins (including the group of cryptophycin 1 and cryptophycin 8); epothilones, eleutherobin, discodermolide, bryostatins, dolostatins, auristatins, tubulysins, cephalostatins; pancratistatin; a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (including mycophenolic acid, tiazofurin, ribavirin, EICAR); Ribonucleotide reductase Inhibitors: (including hydroxyurea, deferoxamine)]; [Pyrimidine analogs: Uracil analogs: (including ancitabine, azacitidine, 6-azauridine, capecitabine, carmofur, cytarabine, dideoxyuridine, doxifluridine, enocitabine, 5-Fluorouracil, floxuridine, ratitrexed); Cytosine analogs: (including cytarabine, cytosine arabinoside, fludarabine); Purine analogs: (including azathioprine, fludarabine, mercaptopurine, thiamiprine, thioguanine)]; folic acid replenisher, frolinic acid};
e). A hormonal therapy: selected from Receptor antagonists: [Anti-estrogen: (including megestrol, raloxifene, tamoxifen); LHRH agonists: (including goscrclin, leuprolide acetate); Anti-androgens: (including bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (including CB 1093, EB 1089 KH 1060, cholecalciferol, ergocalciferol); Photodynamic therapies: (including verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib, bafetinib, bosutinib, cabozantinib, vismodegib, iniparib, ruxolitinib, CYT387, axitinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group of olaparib, niraparib, iniparib, talazoparib, veliparib, CEP 9722 (Cephalon's), E7016 (Eisai's), BGB-290 (BeiGene's), or 3-aminobenzamide;
h). An antibiotic, selected from the group consisting of an enediyne antibiotic (selected from the group of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins andl-methyl-4-phenylpyridinium ion, Cell cycle inhibitors (including staurosporine),
Actinomycins (including Actinomycin D, dactinomycin), amanitins, Bleomycins (including bleomycin A2, bleomycin B2, peplomycin), Anthracyclines (including daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((including Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK©; razoxane; rhizoxin; sizofiran; spirogermanium; tenuazonic acid; triaziquone; 2, 2′,2″-trichlorotriethylamine; trichothecenes (including the group of T-2 toxin, verrucarin A, roridin A and anguidine); urethane, siRNA, antisense drugs;
(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus;
(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem, cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefmninox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (including cefoxitin, cefotetan, cefinetazole), oxacephem (flomoxef, latamoxef);
f). Glycopeptides: bleomycin, vancomycin (including oritavancin, telavancin), teicoplanin (dalbavancin), ramoplanin;
g). Glycylcyclines: tigecycline;
h). β-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
1). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, cnoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). Other antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of p-1-thymidine and/p-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nuclcosides: amantadine, ateviridine, capravirinc, diarylpyrimidines (etravirinc, rilpivirine), delavirdine, docosanol, emivirine, efavirenz, foscamet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). Oh y anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5). The pharmaceutically acceptable salts, acids, derivatives, hydrate or hydrated salt; or a crystalline structure; or an optical isomer, racemate, diastereomer or enantiomer of any of the above drugs.

22. The pharmaceutical composition according to claim 20, wherein the other conjugates for use in the synergistical treatment or prevention of a cancer are selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-lb, Interferon alfa-2a, Interferon alfa-2b, Interferon beta, Interferon beta 1a, Interferon beta Ib, Interferon gamma-Ia, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.
